(19) United States
(12) Patent Application Publication
(10) Pub. No.: US 2024/0124889 A1
(43) Pub. Date: Apr. 18, 2024

(54) COMPOSITIONS AND METHODS FOR THE VECTORED AUGMENTATION OF PROTEIN DESTRUCTION, EXPRESSION AND/OR REGULATION

(71) Applicant: Voyager Therapeutics, Inc., Cambridge, MA (US)

(72) Inventors: Xiao-Qin Ren, Cambridge, MA (US); Jinzhao Hou, Cambridge, MA (US); Steven Paul, Cambridge, MA (US); Kelly Bales

(21) Appl. No.: 17/609,170

(22) PCT Filed: May 7, 2020
(86) PCT No.: PCT/US2020/031853

§ 371 Date: Nov. 5, 2021

Related U.S. Application Data
(60) Provisional application No. 62/844,433, filed on May 7, 2019.
(60) Provisional application No. 62/984,875, filed on Mar. 4, 2020.

(51) Int. Cl. C12N 15/86; A61K 48/00; C07K 16/00; C12N 9/10

(52) U.S. Cl. CPC C12N 15/86; A61K 48/005; C07K 16/00; C12N 9/104; C12Y 203/02; C12N 2750/14143

(57) ABSTRACT

The disclosure provides compositions and methods for the Vectored Augmentation of the Destruction, Expression and/or Regulation of proteins, e.g., VA-DER systems and methods.

### CROSS REFERENCE TO RELATED APPLICATIONS

This application claims the benefit of U.S. Provisional Patent Application No. 62/844,433, filed May 7, 2019, entitled “Compositions and methods for vectored augmentation No. 62/984,875, filed Mar. 4, 2020, entitled “Compositions and methods for vectored augmentation of protein destruction, expression and/or regulation”, the contents of each of which are incorporated herein by reference in their entirety.

### REFERENCE TO THE SEQUENCE LISTING

The present application is being filed along with a Sequence Listing in electronic format. The Sequence Listing file, entitled 20571080PCTSL.txt, was created on May 7, 2020, and is 47,075,659 bytes in size. The information in electronic format of the Sequence Listing is incorporated herein by reference in its entirety.

### FIELD OF THE DISCLOSURE

The disclosure relates to compositions and methods for Vectored Augmentation of the Destruction, Expression and/or Regulation of proteins, i.e., VA-DER.

### BACKGROUND

Methods for targeting the destruction of proteins are known in the art. These include, inter cilia, those involving altering the DNA or RNA encoding the protein or interfering with the translation process by knocking out a gene or mRNA which encodes the protein. Recently, a method termed “Trim-Away” was reported by Clift, et al, (Cell, Volume 172, Issue 7, p 1692-1706.e18, 14 Dec. 2017, the contents of which are incorporated herein by reference in their entirety) whereby endogenous proteins are degraded by utilizing antibodies targeted to the protein to be degraded and the TRIM21 protein, which, as an E3 ubiquitin ligase binds with high affinity to a particular domain of Fc region of antibodies. Once the TRIM21 protein binds an antibody, it facilitates the transfer of the bound antibody and its bound antigen to the ubiquitin-proteasome system where the antibody and its bound antigen are degraded. Clift, et at. demonstrated that this research tool could be used in cell culture and primary human and mouse cells.

The present disclosure goes beyond the benchtop use or application of the Trim-Away tool, Tunable protein expression, degradation and regulation in vivo offers an array of applications in diagnosing, preventing and treating disease. The present disclosure embraces methods for the vectored augmentation of protein destruction, expression and/or regulation, also known herein as VA-DER, which is useful in therapeutics and diagnostics.

### SUMMARY

Described herein are systems and methods for the vectored augmentation of the destruction, expression and/or regulation of proteins, or VA-DER Systems or Vectored Augmentation (VA) methods. VA-DER systems and VA methods as the name implies, exploit vectored delivery, eg., delivery of nucleic acid-based. vector(s), of one or more components of the VA-DER system. VA-DER system components may be vectorized (encoded by a vector or vector genome) or non vectorized (be amino acid based or nucleic acid based). Vectorized (i.e., encoded in a vector) components may include (i) an antibody or fragment or variant thereof, (ii) a payload protein such as the protein TRIM21 or functional equivalent or variant thereof, and/or (iii) a companion or corollary molecule which may be nucleic acid based (e.g., microRNA, aptamer, siRNA, dsRNA, etc.) or which encodes a peptide or protein or which is a peptide or protein. Any of the vectorized components may also be delivered as non-vectorized components, e.g., a protein along with an AAV encoding an antibody, or an antigen along with a lentivirus encoding an antibody, etc.

VA-DER systems and/or methods may comprise one or more vectorized or non-vectorized components.

In some embodiments the vectorized component is an AAV particle comprising a viral genome, wherein the viral genome encodes one or more antibodies.

In some embodiments the vectorized component is an AAV particle comprising a viral genome, wherein the viral genome encodes TRIM21.

In some embodiments the vectorized component is an AAV particle comprising a viral genome, wherein the viral genome encodes one or more companion molecules.

In some embodiments the VA-DER System comprises an AAV vectorized antibody and a protein encoding TRIM21.

In some embodiments the VA-DER System comprises an AAV vectorized antibody and an AAV vectorized TRIM21.

VA-DER system may be vectorized (encoded by a vector or vector genome), and the vectorized (i.e., encoded in a vector) component may include a payload comprising a nucleic acid sequence encoding (i) at least one TRIM2.I protein or TRIM21 protein fragment, (ii) at least one antibody or antibody fragment, and/or (iii) at least one target binding protein or fragment thereof. The vector may be an AAV or variant thereof such as, but not limited to, any of the serotypes listed herein including Table 1. The antibody or antibody fragment may be any of the antibodies listed herein including, but not limited to, those listed in Tables 3-53, an Fe, scFV, nanobody, intrabody, and Fab fragment or combinations thereof. As a non-limiting example, the antibody fragment is used in combination with at least one other different antibody fragment. As a non-limiting example, the antibody fragment is an Fc fragment and the Fe fragment is used in combination with at least one other different antibody fragment. As a non-limiting example, the target binding protein is a tau or tau binding protein.

VA-DER system may be vectorized (encoded by a vector or vector genome), and the vectorized (i.e., encoded in a vector) component may include a chimeric antigen receptor payload comprising a nucleic acid sequence encoding (i) at least one TRIM21 protein or TRIM21 protein fragment, (ii) at least one antibody or antibody fragment, and/or (iii) at least one target binding protein or fragment thereof. The vector may be an AAV or variant thereof such as, but not limited to, any of the serotypes listed herein including Table 1. The antibody or antibody fragment may be any of the antibodies listed herein including, but not limited to, those listed in Tables 3-53, an Fe, scFV, nanobody, intrabody, and Fab fragment or combinations thereof. As a non-limiting example, the antibody fragment is used in combination with at least one other different antibody fragment. As a non-limiting example, the antibody fragment is an Fe fragment and the Fe fragment is used in combination with at least one other different antibody fragment. As a non-limiting example, the target binding protein is a tau or tau binding protein.

### BRIEF DESCRIPTION OF TIM DRAWINGS

The foregoing and other objects, features, and advantages will be apparent from the following description of particular embodiments of the disclosure, as illustrated in the accompanying drawings. The drawings are not necessarily to scale, emphasis instead being placed upon illustrating the principles of various embodiments of the disclosure.

FIG. 1 is a schematic of vectored antibody delivery.

FIG. 2 is a schematic of a viral genome.

FIG. 3 is a schematic of payload regions. Figure discloses “5xG4S” as SEQ ID NO: 32689 or SEQ ID NO: 1728.

### DETAILED DESCRIPTION

### I. Compositions

### VA-DER Systems and Methods

The present disclosure involves the exploitation of the TRIM21 protein and the TRIM21-associated pathway for the Vectored Augmentation (VA) of protein Destruction, Expression and/or Regulation (DER), i.e., VA-DER systems and methods.

TRIM21 also known as Sjogren Syndrome Antigen A1; SSA1; SICCA Syndrome Antigen A; SSA; Autoantigen Ro/SSA, 52-KD; and 8052, encodes a 52 kDa protein of 475-amino acids. It has multiple N-terminal zinc finger motifs, a central leucine zipper, and a potential N-glycosylation site. It contains an N-terminal RING finger that has E3 ligase activity, followed by a B box, 2 coiled-coil regions, and a long C-terminal PRYSPRY domain that binds IgCB Fc fragments.

The present disclosure provides a means by which the level or amount of any protein (peptide, antigen, polypeptide, antibody, fusion protein, conjugate, chimeric antigen receptor, or any biomolecule which may be bound by an antibody, including the antibody itself) in a cell may be augmented by taking advantage of the properties of the TREM21 protein.

The vector augmented systems of the present disclosure comprise one or more components, one of which is TRIM21 (either as a protein or encoded in a nucleic acid). This TRIM21 effector may be delivered in vectored form alone or in combination with other molecules such as antibodies, other proteins, or nucleic acid-based molecules. In doing so, TRIM21 allows for augmentation of the level of an antibody to which it binds, thereby facilitating its trafficking to the proteasome and ultimate destruction; or the augmentation of the level of the antigen to which the targeted antibody is bound.

The VA-DER TRIM21 systems may be utilized in the area of regulating the immune system by binding to one or more antibodies or antibody-bound receptors such as chimeric antigen receptors (CARs).

As a component of the VA-DER systems and or methods of the disclosure, TRIM21 may be delivered as a protein or as an encoded nucleic acid by any vector or plasmid-based delivery system. Such systems include retroviral vehicles, retroviral particles, lentiviral vehicles, lentiviral particles, adenoviruses, adeno-associated viruses (AAV), nanoparticles, liposomes and the like. These delivery vehicles are described in more detail here.

### Retroviral Vehicles and Retroviral Particles (γ-Retroviral Vectors)

In some embodiments, retroviral vehicles and retroviral particles may be used to deliver the VA-DER compositions or components for delivering functional proteins, nucleic acids, antibodies and/or antibody-based compositions of the present disclosure. Retroviral vectors (RVs) allow the permanent integration of a transgene in target cells. In addition to lentiviral vectors based on complex HIV-1/2, retroviral vectors based on simple gamma-retroviruses have been widely used to deliver therapeutic genes and demonstrated clinically as one of the most efficient and powerful gene delivery systems capable of transducing a broad range of cell types. Example species of Gamma retroviruses include the murine leukemia viruses (MLVs) and the feline leukemia viruses (FeLV).

In some embodiments, gamma-retroviral vectors derived from a mammalian gamma-retrovirus such as murine leukemia viruses (MLVs), are recombinant. The MLA/families of gamma retroviruses include the ecotropic, amphotropic, xenotropic and polytropic subfamilies. Ecotropic viruses are able to infect only murine cells using mCAT-1 receptor. Examples of ecotropic viruses are Moloney MIN and AKV. Amphotropic viruses infect murine, human and other species through the Pit-2 receptor. One example of an amphotropic virus is the 4070A virus. Xenotropic and polytropic viruses utilize the same (Xpr1) receptor but differ in their species tropism. Xenotropic viruses such as NZB-9-1 infect human and other species but not murine species, whereas polytropic viruses such as focus-forming viruses (MCF) infect murine, human and other species.

Gamma-retroviral vectors may be produced in packaging cells by co-transfecting the cells with several plasmids including one encoding the retroviral structural and enzymatic (gag-poi) polyprotein, one encoding the envelope (env) protein, and one encoding the vector mRNA comprising polynucleotide encoding the compositions of the present disclosure that is to be packaged in newly formed viral particles.

In some aspects, the recombinant gamma-retroviral vectors are pseudotyped with envelope proteins from other viruses. Envelope glycoproteins are incorporated in the outer lipid layer of the viral particles which can increase/alter the cell tropism. Exemplary envelop proteins include the gibbon ape leukemia virus envelope protein (GAIN) or vesicular stomatitis virus G protein (VSV-G), or Simian endogenous retrovirus envelop protein, or Measles Virus H and F proteins, or Human immunodeficiency virus gp120 envelop protein, or coral vesiculovirus envelop protein (See, e.g., U.S. application publication NO.: 2012/164118; the contents of which are incorporated herein by reference in its entirety). In other aspects, envelope glycoproteins may be genetically modified to incorporate targeting/binding ligands into gamma-retroviral vectors, binding ligands including, but not limited to, peptide ligands, single chain antibodies and growth factors (Wackier et al., Nat. Rev. Genet. 2007, 8(8):573-587; the contents of which are incorporated herein by reference in its entirety). These engineered glycoproteins can retarget vectors to cells expressing their corresponding target moieties. In other aspects, a “molecular bridge” may be introduced to direct vectors to specific cells. The molecular bridge has dual specificities: one end can recognize viral glycoproteins, and the other end can bind to the molecular determinant on the target cell. Such molecular bridges, for example ligand-receptor, avidin-biotin, and chemical conjugations, monoclonal antibodies and engineered fusogenic proteins, can direct the attachment of viral vectors to target cells for transduction (Yang et al., Biotechnol. Bioeng., 2008, 101(2): 357-368; and Maetzig et al., Viruses, 2011, 3, 677-713; the contents of each of which are incorporated herein by reference in their entirety).

In some embodiments, the recombinant gammaretroviral vectors are self-inactivating (SIN) gammaretroviral vectors. The vectors are replication incompetent. SIN vectors may harbor a deletion within the 3′ U3 region initially comprising enhancer/promoter activity. Furthermore, the 5′ U3 region may be replaced with strong promoters (needed in the packaging cell line) derived from Cytomegalovirus or RSV, or an internal promotor of choice, and/or an enhancer element. The choice of the internal promotors may be made according to specific requirements of gene expression needed for a particular purpose.

In some embodiments, polynucleotides encoding the bio functional antibodies and/or antibody-based compositions are inserted within the recombinant viral genome. The other components of the viral mRNA of a recombinant gammaretroviral vector may be modified by insertion or removal of naturally occurring sequences (e.g., insertion of an IRES, insertion of a heterologous polynucleotide encoding a polypeptide or inhibitory nucleic acid of interest, shuffling of a more effective promoter from a different retrovirus or virus in place of the wild-type promoter and the like). In some examples, the recombinant gammaretroviral vectors may comprise modified packaging signal, and/or primer binding site (PBS), and/or 5′-enhancer/promoter elements in the U3-region of the 5′-long terminal repeat (LTR), and/or 3′-SIN elements modified in the U3-region of the 3′-LTR. These modifications may increase the titers and the ability of infection.

Gamma-retroviral vectors suitable for delivering functional antibodies and/or antibody-based compositions of the present disclosure may be selected from those disclosed in U.S. Pat. Nos. 8,828,718; 7,585,676; 7,351,585; U.S. application publication NO.: 2007/048285; PCT application publication NOs.: WO2010/113037; WO2014/121005; WO2015/056014; and EP Pat. NOs; EP1757702; EP1757703 (the contents of each of which are incorporated herein by reference in their entirety).

### Lentiviral Vehicles and Lentiviral Particles

In some embodiments, lentiviral vehicles and lentiviral particles may be used as delivery modalities. In some embodiments, lentiviral vehicles and lentiviral particles may be used to deliver the VA-DER compositions or components for delivering functional proteins, nucleic acids, antibodies and/or antibody-based compositions of the present disclosure.

Lentiviruses are subgroup of the Retroviridae family of viruses, named because reverse transcription of viral RNA genomes to DNA is required before integration into the host genome. As such, the most important features of lentiviral vehicles and lentiviral particles are the integration of their genetic material into the genome of a target/host cell. Some examples of lentivirus include the Human Immunodeficiency Viruses: HIV-1 and HIV-2, the Simian Immunodeficiency Virus (SIV), feline immunodeficiency virus (FPV), bovine immunodeficiency virus (BIN), Jembrana Disease Virus (JDV), equine infectious anemia virus (E V), equine infectious anemia virus, visna maedi and caprine arthritis encephalitis virus (CAEV).

Typically, lentiviral particles making up the gene delivery vehicle are replication defective on their own (also referred to as “self-inactivating”). Lentiviruses are able to infect both dividing and non-dividing cells by virtue of the entry mechanism through the intact host nuclear envelope (Naldini L et al., Cure. Opin. Biotechnol, 1998, 9: 457-463). Recombinant lentiviral vehicles and lentiviral particles have been generated by multiply attenuating the HIV virulence genes, for example, the genes Env, Vif, Vpr, Vpu, Nef and Tat are deleted making the vector biologically safe. Correspondingly, lentiviral vehicles, for example, derived from HIV-1/HIV-2 can mediate the efficient delivery, integration and long-term expression of transgenes into non-dividing cells.

Lentiviral particles may be generated by co-expressing the virus packaging elements and the vector genome itself in a producer cell such as human HEK293T cells. These elements are usually provided in three or four separate plasmids. The producer cells are co-transfected with plasmids that encode lentiviral components including the core (i.e. structural proteins) and enzymatic components of the virus, and the envelope protein(s) (referred to as the packaging systems), and a plasmid that encodes the genome including a foreign transgene, to be transferred to the target cell, the vehicle itself (also referred to as the transfer vector). In general, the plasmids or vectors are included in a producer cell line. The plasmids/vectors are introduced via transfection, transduction or infection into the producer cell line. Methods for transfection, transduction or infection are well known by those of skill in the art. As non-limiting example, the packaging and transfer constructs can be introduced into producer cell lines by calcium phosphate transfection, lipofection or electroporation, generally together with a dominant selectable marker, such as neo, DHFR, Gin synthetase or ADA, followed by selection in the presence of the appropriate drug and isolation of clones.

The producer cell produces recombinant viral particles that contain the foreign gene, for example, the payload of the present disclosure. The recombinant viral particles are recovered from the culture media and titrated by standard methods used by those of skill in the art. The recombinant lentiviral vehicles can be used to infect target cells.

Cells that can be used to produce high-titer lentiviral particles may include, but are not limited to, HEK293T cells, 293G cells, STAR cells (Relander et al., Mol Ther., 2005, 11: 452-459), FreeStyle™ 293 Expression System (ThermoFisher, Waltham, MA), and other HEK293T-based producer cell lines (e.g., Stewart et al., Hum Gene Ther. 2011, 22 (3):357369; Lee et al., Biotechnol Bioeng, 2012, 10996): 1551-1560; Throm et al., Blood. 2009, 113(21): 5104-5110; the contents of each of which are incorporated herein by reference in their entirety).

In some aspects, the envelope proteins may be heterologous envelop proteins from other viruses, such as the G protein of vesicular stomatitis virus (VSV G) or baculoviral gp64 envelop proteins. The VSV-G glycoprotein may especially be chosen among species classified in the vesiculovirus genus: Carajas virus (MY), Chandipura virus (CHPV), Cocal virus (COCV), Isfahan virus (ISFV), Maraba virus (MALAY), Pity virus (PIRYV), Vesicular stomatitis Alagoas virus (VSAV), Vesicular stomatitis Indiana virus (VSTV) and Vesicular stomatitis New Jersey virus (VSNJV) and/or stains provisionally classified in the vesiculovirus genus as Grass carp rhabdovirus, BeAn 157575 virus (BeAn 157575), Boteke virus (BTKV), Calchaqui virus (CQIV); Eel virus American (EVA), Gray Lodge virus (GLOV), Jurona virus (JURY), Klamath virus (KLAV), Kwatta virus (KWAV), La Jaya virus (LJV), Malpais Spring virus (MSPV), Mount Elgon bat virus (MEBV), Perinet virus (PERV), Pike fry rhabdovirus (PERV), Porton virus (PORV), Radi virus (RADIV), Spring viremia of carp virus (SVCV), Tupaia virus (TUPV), Ulcerative disease rhabdovirus (UDRV) and Yug Bogdanovac virus (YBV). The gp64 or other baculoviral env protein can be derived from Autographa californica nucleopolyhedrovirus (AcMNPV), Anagrapha falcifera nuclear polyhedrosis virus, Bombyx more nuclear polyhedrosis virus, Choristoneura fumiferana nucleopolyhedrovirus, Orgyia pseudotsugata single capsid nuclear polyhedrosis virus, Epiphyas postvittana nucleopolyhedrovinis, Hyphantria cunea nucleopolyhedrovirus, Galleria mellonella nuclear polyhedrosis virus, Dhori virus, Thogoto virus, Antheraea pemyi nucleopolyhedrovirus or Batken virus.

Other elements provided in lentiviral particles may comprise retroviral LTR (long-terminal repeat) at either 5′ or 3′ terminus, a retroviral export element, optionally a lentiviral reverse response element (RRE), a promoter or active portion thereof, and a locus control region (LCR) or active portion thereof.

Methods for generating recombinant lentiviral particles are discussed in the art, for example, U.S. Pat. Nos. 8,846,385; 7,745,179; 7,629,153; 7,575,924; 7,179,903; and 6,808,905; the contents of each of which are incorporated herein by reference in their entirety.

Lentivirus vectors used may be selected from, but are not limited to pLVX, pLenti, pLenti6, pLJM1, FUGW, pWPXL, pWPI, pLenti CMV euro REST, pLJM1-EGFP, pULTRA, pInducer20, pHIV-EGFP, pCW57.1, pTRPE, pELPS, pRRL, and pLionII.

Lentiviral vehicles are plasmid-based or virus-based and are known in the art (See, U.S. Pat. Nos. 9,260,725; 9,068,199; 9,023,646; 8,900,858; 8,748,169; 8,709,799; 8,420,104; 8,329,462; 8,076,106; 6,013,516; and 5,994,136; the contents of each of which are incorporated herein by reference in their entirety).

### Adeno-Associated Viruses (AAVs) and AAV Particles

In some embodiments, AAV and AAV particles may be used to deliver the VA-DER compositions or components for delivering functional proteins, nucleic acids, antibodies and/or antibody-based compositions of the present disclosure.

According to the present disclosure, compositions for delivering functional antibodies and/or antibody-based compositions by adeno-associated viruses (AAVs) as components of VA-DER systems are provided.

AAV particles of the disclosure may be provided via any of several routes of administration, to a cell, tissue, organ, or organism, in vivo, ex vivo, or in vitro.

As used herein, an “AAV particle” is an AAV which comprises a viral genome with at least one payload region and at least one inverted terminal repeat (ITR) region.

As used herein, “viral genome” or “vector genome” refers to the nucleic acid sequence(s) encapsulated in an AAV particle. Viral genomes comprise at least one payload region encoding polypeptides of the disclosure, e.g., antibodies, antibody-based compositions or fragments thereof.

As used herein, a “payload” or “payload region” is any nucleic acid molecule which encodes one or more polypeptides of the disclosure. In some embodiments, the payload may encode TRIM21 or a variant thereof. At a minimum, a payload region comprises nucleic acid sequences that encode a protein, polypeptide, antibody, an antibody-based composition, or a fragment thereof but may also optionally comprise one or more functional or regulatory elements to facilitate transcriptional expression and/or polypeptide translation. Payloads may also be nucleic acid based and not encode a protein, e.g., miRNA, siRNA, aptamers, etc.

In some embodiments, AAV particles, viral genomes and/or payloads of the disclosure, and the methods of their use may be as described in WO2017189963, the contents of which are herein incorporated by reference in their entirety.

The nucleic acid sequences and polypeptides disclosed herein may be engineered to contain modular elements and/or sequence motifs assembled to enable expression of the antibodies or antibody-based compositions of the VA-Milk systems of the disclosure. In some embodiments, the nucleic acid sequence comprising the payload region may comprise one or more of a promoter region, an intron, a Kozak sequence, an enhancer, or a polyadenylation sequence. Payload regions of the disclosure typically encode antibodies or antibody-based compositions, which may include an antibody heavy chain domain, an antibody light chain domain, both antibody heavy and light chain domains, or fragments of the foregoing in combination with each other or in combination with other polypeptide moieties. In some cases, payload regions may also encode one or more linkers or joining regions between antibody heavy and light chain domains or fragments. The order of expression, structural position, or concatemer count (heavy chain, light chain, or linker) may be different within or among different payload regions. The identity, position and number of linkers expressed by payload regions may also vary.

The payload regions of the disclosure may be delivered to one or more target cells, tissues, organs, or organisms within the viral genome of an AAV particle.

Viruses of the Parvoviridae family are small non-enveloped icosahedral capsid viruses characterized by a single stranded DNA genome. Parvoviridae family viruses consist of two subfamilies: Parvovirinae, which infect vertebrates, and Densovirinae, which infect invertebrates. Due to its relatively simple structure, easily manipulated using standard molecular biology techniques, this virus family is useful as a biological tool. The genome of the virus may be modified to contain a minimum of components for the assembly of a functional recombinant virus, or viral particle, which is loaded with or engineered to express or deliver a desired payload, which may be delivered to a target cell, tissue, organ, or organism.

The parvoviruses and other members of the Parvoviridae family are generally described in Kenneth I. Berns, “Parvoviridae: The Viruses and Their Replication,” Chapter 69 in FIELDS VIROLOGY (3d Ed. 1996), the contents of which are incorporated by reference in their entirety.

The Parvoviridae family comprises the Dependovirus genus which includes adeno associated viruses (AAV) capable of replication in vertebrate hosts including, but not limited to, human, primate, bovine, canine, equine, and ovine species.

The AAV vector genome is a linear, single-stranded DNA (ssDNA) molecule approximately 5,000 nucleotides (nt) in length. The AAV viral genome can comprise a payload region and at least one inverted terminal repeat (ITR) or ITR region. ITRs traditionally flank the coding nucleotide sequences for the non-structural proteins (encoded by Rep genes) and the structural proteins (encoded by capsid genes or Cap genes). While not wishing to be bound by theory, an AAV viral genome typically comprises two ITR sequences. The AAV vector genome comprises a characteristic T-shaped hairpin structure defined by the self-complementary terminal 145 nt of the 5′ and 3′ ends of the ssDNA which form an energetically stable double stranded region. The double stranded hairpin structures comprise multiple functions including, but not limited to, acting as an origin for DNA replication by functioning as primers for the endogenous DNA polymerase complex of the host viral replication cell.

In addition to the encoded heterologous payload, AAV vectors may comprise the viral genome, in whole or in part, of any naturally occurring and/or recombinant AAV serotype nucleotide sequence or variant. AAV variants may have sequences of significant homology at the nucleic acid (genome or capsid) and amino acid levels (capsids), to produce constructs which are generally physical and functional equivalents, replicate by similar mechanisms, and assemble by similar mechanisms. Chiorini et al., J. Vir. 71: 6823-33(1997); Srivastava et al. J. Vir. 45:555-64 (1983); Chiorini et al., J. Vir. 73:1309-1319 (1999); Rutledge et al., J. Vir. 72:309-319 (1998); and Wu et al., J. Vir. 74: 8635-47 (2000), the contents of each of which are incorporated herein by reference in their entirety.

In some embodiments, AAV particles of the present disclosure are recombinant AAV viral vectors which are replication defective and lacking sequences encoding functional Rep and Cap proteins within their viral genome. These defective AAV vectors may lack most or all parental coding sequences and essentially carry only one or two AAV ITR sequences and the nucleic acid of interest for delivery to a cell, a tissue, an organ, or an organism.

In some embodiments, the viral genome of the AAV particles of the present disclosure comprise at least one control element which provides for the replication, transcription, and translation of a coding sequence encoded therein. Not all of the control elements need always be present as long as the coding sequence is capable of being replicated, transcribed, and/or translated in an appropriate host cell. Non-limiting examples of expression control elements include sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

According to the present disclosure, AAV particles for use in therapeutics and/or diagnostics comprise a virus that has been distilled or reduced to the minimum components necessary for transduction of a nucleic acid payload or cargo of interest. In this manner, AAV particles are engineered as vehicles for specific delivery while lacking the deleterious replication and/or integration features found in wild-type viruses.

AAV vectors of the present disclosure may be produced recombinantly and may be based on adeno-associated virus (AAV) parent or reference sequences. As used herein, a “vector” is any molecule or moiety which transports, transduces, or otherwise acts as a carrier of a heterologous molecule such as the nucleic acids described herein.

In addition to single stranded AAV viral genomes (e.g., ssAAVs), the present disclosure also provides for self-complementary AAV (scAAVs) viral genomes. scAAV vector genomes contain DNA strands which anneal together to form double stranded DNA. By skipping second strand synthesis, scAAVs allow for rapid expression in the cell.

In some embodiments, the AAV particle of the present disclosure is an scAAV.

In some embodiments, the AAV particle of the present disclosure is an ssAAV,

Methods for producing and/or modifying AAV particles are disclosed in the art such as pseudotyped AAV vectors (PCT Patent Publication Nos. WO200028004; WO200123001; WO2004112727; WO2005005610; and WO2005072364, the content of each of which is incorporated herein by reference in its entirety).

AAV particles may be modified to enhance the efficiency of delivery. Such modified AAV particles can be packaged efficiently and be used to successfully infect the target cells at high frequency and with minimal toxicity. In some embodiments, the capsids of the AAV particles are engineered according to the methods described in US Publication Number US20130195801, the contents of which are incorporated herein by reference in their entirety.

In some embodiments, the AAV particles comprising a payload region encoding the polypeptides of the disclosure may be introduced into mammalian cells.

### AAV Serotypes

AAV particles of the present disclosure may comprise or be derived from any natural or recombinant AAV serotype. According to the present disclosure, the AAV particles may utilize or be based on a serotype or include a peptide selected from any of the following VOY101, VOY201, AAVPHP.B (PHP.B), AAVPHP.A. (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHRB-DGT, AAVPHP,B-EST, AAVPHP,B-GGT,AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NOT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP,B-TTP, AAVPHP.S/G2 A12, AAVG2A15/G2A3 (G2A3), AAVG2B4 (G2B4), AAVG2B5 (G2B5), PHP.S, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61., AAV9.68, AAV9.84, AAV9.9, AAV10, AAV11, AAV12, AAV16.3, AAV24.1, AAV27.3, AAV42.12, AAV42-11), AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAA/1-7/rh.48, AAV1-8/rh.49, AAV2-15/rh.62, AAV2-3/rh.61., AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb. 1, AAV29.5/bb 0.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV1611.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi 0.2, AAVrh.60, AAVrh.44, AAVrh.65, AAVrh.55, AAVrh.47, AAVrh.69, AAVrh.45, AAVrh.59, AAVhu.12, AAVH6, AAVLK.03, AAVH-1/hu.1, AAVH-5/hu.3, AAVLG-10/rh.40, AAVLG-4/rh. 38, AAVLG-9/hu. 39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAA/hu.11, AAV hu.13, AAVhu.15, AAVhu.16, AAVhu.17, AAVhu.18, AAVhu.20, AAVhu.21, AAVhu.22, AAVhu.23,2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.4413.2, AAVhu.4413.3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh 36, AAVrh.37, AAVrh.37R2, AAVrh.38, AAVrh.39, AAVrh 40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2,5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62, AAV, BNP63, AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/1111.21, AAV54.4R/hu.27, AAV46.2/1111,28, AAV46.6/hu.29, AAV128.1/huA3, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8,AAV CBr-B7.3, AAV CBr-B7.4, AAV CBr-E1,AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV AAV CHt-3, AAV CHt-6.1, AAV CHt-6.10, AAV CHt-6.5, AAV CHI-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-111, AAV CKd-H2, AAV CKd-H3, AAV CKd-414, AAV CKd-115, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLA1-1, AAV Clv1-10, AAV CLv1-2, AAV CLv-12, AAV CLv1-3, AAV CLv-13, AAV CLv1-4, AAV Clv1-7, AAV Clv1-8, AAV AAV CLv-2, AAV CLv-3, AAV CLv-4,AAV AAV CLv-8, AAV CLv-D1, AAV CLv-D2, AAV CLv-D3, AAV CLv-D4, AAV CLv-D5, AAV CLv-D6, AAV CLv-D7, AAV AAV CLv-E1, AAV CLv-K1, AAV CLv-K3, AAV CLv-K6, AAV CLv-L4, AAV CLv-L5, AAV CLv-L6, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV AAV AAV AAV CLv-R8, AAV CLv-R9, AAV CSp-1, AAV CSp-10, AAV CSp-11, AAV CSp-2, AAV CSp-3, AAV CSp-4,AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAVhu.48R3, AAV.VR-355, AAV3B, AAV4, AAV5, AAVF1/HSC1, AAVF1.1/HSC11, AAVF12/HSC12, AAVF13/HSC13, AAVF14/HSC14, AAVF15/HSC15 AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9 and variants thereof.

In some embodiments, the AAV serotype may be, or have, a sequence as described in United States Publication No. US20030138772, the contents of which are herein incorporated by reference in their entirety, such as, but not limited to, AAV1 (SEQ ID NO: 6 and 64 of US20030138772), AAV2 (SEQ ID NO: 7 and 70 of US20030138772), AAV3 (SEQ ID NO: 8 and 71 of US20030138772), AAV4 (SEQ ID NO: 63 of US20030138772), AAV5 (SEQ ID NO: 114 of US20030138772), AAV6 (SEQ ID NO: 65 of US20030138772), AAV7 (SEQ ID NO: 1-3 of US20030138772), AAV8 (SEQ ID NO: 4 and 95 of US20030138772), AAV9 (SEQ ID NO: 5 and 100 of US20030138772), AAV10 (SEQ ID NO: 117 of US20030138772), AAV11 (SEQ ID NO: 118 of US20030138772), AAV12 (SEQ ID NO: 119 of US20030138772), AAVrh10 (amino acids 1 to 738 of SEQ ID NO: 81 of US20030138772), AAV16.3 (US20030138772 SEQ. ID NO: 10), AAV29.3/bb.1 (US20030138772 SEQ ID NO: 11), AAV29.4 (US20030138772 SEQ ID NO: 12), AAV29.5/bb.2 (US20030138772 SEQ ID NO: 13), AAV1.3 (US20030138772 SEQ ID NO: 14), AAV13.3 (US20030138772 SEQ ID NO: 15), AAV24.1 (US20030138772 SEQ ID NO: 16), AAV27.3 (US20030138772 SEQ ID NO: 17), AAV7.2 (US20030138772 SEQ ID NO: 18), AAVC1 (0520030138772 SEQ ID NO: 19), AAVC3 (US20030138772 SEQ ID NO: 20), AAS' C5 (US20030138772 SEQ ID NO: 21), AAVF1 (US20030138772 SEQ ID NO: 22), AAVF3 (US20030138772 SEQ ID NO: 23), AAVF5 (US20030138772 SEQ ID NO: 24), AAVH6 (US20030138772 SEQ ID NO: 25), AAVH2 (US20030138772 SEQ ID NO: 26), AAV42-8 (US20030138772 SEQ ID NO: 27), AAV42-15 (US20030138772 SEQ ID NO: 28), AAV42-5b (US20030138772 SEQ ID NO: 29), AAV42-1b (US20030138772 SEQ ID NO: 30), AAV42-13 (US20030138772 SEQ ID NO: 31), AAV42-3a. (US20030138772 SEQ ID NO: 32), AAV42-4 (US20030138772 SEQ ID NO: 33), AAV42-5a (US20030138772 SEQ ID NO: 34), AAV42-10 (US20030138772 SEQ ID NO: 35), AAV42-3b (US20030138772 SEQ ID NO: 36), AAV42-11 (US20030138772 SEQ ID NO: 37), AVV42-6b (US20030138772 SEQ ID NO: 38), AAV43-1 (US20030138772 SEQ ID NO: 39), AAV43-5 (US20030138772 SEQ ID NO: 40), AAV43-12 (US20030138772 SEQ ID NO: 41), AAV43-20 (US20030138772 SEQ ID NO: 42), AAV43-21 (US20030138772 SEQ ID NO: 43), AAV43-23 (US20030138772 SEQ ID NO: 44), AAV43-25 (11520030138772 SEQ ID NO: 45), AAV44.1 (US20030138772 SEQ ID NO: 46), AAV44.5 (US20030138772 SEQ ID NO: 47), AAV223.1 (US20030138772 SEQ ID NO: 48), AAV223.2 (US20030138772 SEQ ID NO: 49), AAV223.4 (US20030138772 SEQ ID NO: 50), AAV223.5 (US20030138772 SEQ ID NO: 51), AVV223.6 (US20030138772 SEQ ID NO: 52), AAV223.7 (US20030138772 SEQ ID NO: 53), AAVA3.4 (US20030138772 SEQ ID NO: 54), AAVA3.5 (US20030138772 SEQ ID NO: 55), AAVA3.7 (US20030138772 SEQ ID NO: 56), AAVA3.3 (US20030138772 SEQ ID NO: 57), AAV42.12 (US20030138772 SEQ ID NO: 58), AAV44.2 (US20030138772 SEQ ID NO: 59), AAV42-2 (US20030138772 SEQ ID NO: 9), or variants thereof.

In some embodiments, the AAV serotype may be, or have, a sequence as described in U.S. Publication Ser. No. 05/201,50159173, the contents of which are herein incorporated by reference in their entirety, such as, but not limited to, AAV2 (SEQ ID NO: 7 and 23 of US20150159173), rh20 (SEQ ID NO: 1 of US20150159173), rh32/33 (SEQ ID NO: 2 of US20150159173), rh39 (SEQ ID NO: 3, 20 and 36 of US20150159173), rh46 (SEQ ID NO: 4 and 22 of US20150159173), rh73 (SEQ NO: 5 of US20150159173), rh74 (SEQ NO: 6 of US20150159173), AAV6.1 (SEQ ID NO: 29 of US20150159173), rh.8 (SEQ ID NO: 41 of US20150159173), rh.48.1 (SEQ ID NO: 44 of US20150159173), hu.44 (SEQ ID NO: 45 of US20150159173), hu.29 (SEQ ID NO: 42 of US20150159173), hu.48 (SEQ ID NO: 38 of US20150159173), rh54 (SEQ ID NO: 49 of US20150159173), AAV2 (SEQ ID NO: 7 of US20150159173), cy.5 (SEQ ID NO: 8 and 24 of US20150159173), rh.10 (SEQ ID NO: 9 and 25 of US20150159173), rh.13 (SEQ ID NO: 10 and 26 of US20150159173), AAV1 (SEQ ID NO: 11 and 27 of US20150159173), AAV3 (SEQ ID NO: 12 and 28 of US20150159173), AAV6 (SEQ ID NO: 13 and 29 of US20150159173), AAV7 (SEQ ID NO: 14 and 30 of US20150159173), AAV8 (SEQ ID NO: 15 and 31 of US20150159173), hu.13 (SEQ ID NO: 16 and 32 of US20150159173), hu.26 (SEQ ID NO: 17 and 33 of US20150159173), hu.37 (SEQ ID NO: 18 and 34 of US20150159173), hu.53 (SEQ ID NO: 19 and 35 of US20150159173), rh.43 (SEQ ID NO: 21 and 37 of US20150159173), rh2 (SEQ ID NO: 39 of US20150159173), rh.37 (SEQ ID NO: 40 of US20150159173), rh.64 (SEQ ID NO: 43 of US20150159173), rh.48 (SEQ ID NO: 44 of US20150159173), ch.5 (SEQ ID NO 46 of US20150159173), rh.67 (SW 1D NO: 47 of US20150159173), rh.58 (SEQ ID NO: 48 of US20150159173), or variants thereof including, but not limited to Cy5R1, Cy5R2, Cy5R3, Cy5R4, rh.13R, rh.37R2, rh.2R, rh.8R, rh.48.1, rh.48.2, rh.48.1.2, hu.44R1, hu.44R2, hu.44R3, hu.29R, ch.5R1, rh64R1, rh64R2, AAV6.2, AAV6.1, AAV6.12, hu.48R1, hu.48R2, and hu.48R3.

In some embodiments, the AAV serotype may be, or have, a sequence as described in U.S. Pat. No. 7,198,951, the contents of which are herein incorporated by reference in their entirety, such as, but not limited to, AAV9 (SEQ ID NO: 1-3 of U.S. Pat. No. 7,198,951), AAV2 (SEQ ID NO: 4 of U.S. Pat. No. 7,198,951), AAV1 (SEQ ID NO: 5 of U.S. Pat. No. 7,198,951), AAV3 (SEQ ID NO: 6 of U.S. Pat. No. 7,198,951), and AAV8 (SEQ ID NO: 7 of U.S. Pat. No. 7,198,951).

In some embodiments, the AAV serotype may be, or have, a mutation in the AAV9 sequence as described by N Pulicherla et al. (Molecular Therapy 19(6):1070-1078 (2011), herein incorporated by reference in its entirety), such as but not limited to, AAV9.9, AAV9.11, AAV9.13, AAV9,16, AAV9.24, AAV9.45, AAV9.47, AAV9.61, AAV9,68, AAV9.84.

In some embodiments, the AAV serotype may be, or have, a sequence as described in U.S. Pat. No. 6,156,303, the contents of which are herein incorporated by reference in their entirety, such as, but not limited to, AAV3B (SEQ ID NO: 1 and 10 of U.S. Pat. No. 6,156,303), AAV6 (SEQ ID NO: 2, 7 and 11 of U.S. Pat. No. 6,156,303), AAV2 (SEQ ID NO: 3 and 8 of U.S. Pat. No. 6,156,303), AAV3A (SEQ ID NO: 4 and 9, of U.S. Pat. No. 6,156,303), or derivatives thereof.

In some embodiments, the AAV serotype may be, or have, a sequence as described in United States Publication No. US20140359799, the contents of which are herein incorporated by reference in their entirety, such as, but not limited to, AAV8 (SEQ ID NO: 1 of US20140359799), AAVDJ (SEQ ID NO: 2 and 3 of US20140359799), or variants thereof

In some embodiments, the serotype may be AAVDJ or a variant thereof, such as AAVDJ8 (or AAV-DJ8), as described by Grimm et al. (Journal of Virology 82(12): 5887-5911 (2008), herein incorporated by reference in its entirety). The amino acid sequence of AAVDJ8 may comprise two or more mutations in order to remove the heparin binding domain (HBD). As a non-limiting example, the AAV DJ sequence described as SEQ ID NO: 1 in U.S. Pat. No. 7,588,772, the contents of which are herein incorporated by reference in their entirety, may comprise two mutations: (1) R587Q where arginine (R; Arg) at amino acid 587 is changed to glutamine (Q; Gln) and (2) 85901 where arginine (R; Arg) at amino acid 590 is changed to threonine (T; Thr). As another non-limiting example, may comprise three mutations: (1) K406R where lysine (K; Lys) at amino acid 406 is changed to arginine (R; Arg), (2) R587Q where arginine (R; Arg) at amino acid 587 is changed to glutamine (Q; Gin) and (3) R590T where arginine (R; Arg) at amino acid 590 is changed to threonine (T; Thr).

In some embodiments, the AAV serotype may be, or have, a sequence of AAV4 as described in International Publication No. WO1998011244, the contents of which are herein incorporated by reference in their entirety, such as, but not limited to AAV4 (SEQ ID NO: 1-20 of WO1998011244).

In some embodiments, the AAV serotype may be, or have, a mutation in the AAV2 sequence to generate AAV2G9 as described in International Publication No. WO2014144229 and herein incorporated by reference in its entirety.

In some embodiments, the AAV serotype may be, or have, a sequence as described in International Publication No. WO2005033321, the contents of which are herein incorporated by reference in their entirety, such as, but not limited to AAV3-3 (SEQ ID NO: 217 of WO2005033321), AAV1 (SEQ ID NO: 219 and 202 of WO2005033321), AAV106.1/hu.37 (SEQ ID NO: 10 of WO2005033321), AAV114.3/hu.40 (SEQ ID NO: 11 of WO2005033321), AAV127.2/hu.41 (SEQ ID NO:6 and 8 of WO2005033321), AAV128.3/hu.44 (SEQ ID NO: 81 of WO2005033321), AAV130.4/hu.48 (SEQ ID NO: 78 of WO2005033321), AAV145.1/hu.53 (SEQ ID NO: 176 and 177 of WO2005033321), AAV145.6/hu.56 (SEQ ID NO: 168 and 192 of WO2005033321), AAV16.12/hu.11 (SEQ ID NO: 153 and 57 of WO2005033321), AAV16.8/hu.10 (SEQ ID NO: 156 and 56 of WO2005033321), AAV161.10/hu.60 (SEQ ID NO: 170 of WO2005033321), AAV161.6/hu.61 (SEQ ID NO: 174 of WO2005033321), AAV1-7/rh.48 (SEQ ID NO: 32 of WO2005033321), AAV1-8/rh.49 (SEQ ID NOs: 103 and 25 of WO2005033321), AAV2 (SEQ ID NO: 211 and 221 of WO2005033321), AAV2-15/rh.62 (SEQ ID No: 33 and 114 of W(0D2005033321), AAV2-3/rh.61 (SEQ ID NO: 21 of WO2005033321), AAV2-4/rh.50 (SEQ ID NO: 23 and 108 of WO2005033321), AAV2-5/rh.51 (SEQ ID NO: 104 and 22 of WO2005033321), AAV3J/hu.6 (SEQ ID NO: 5 and 84 of WO2005033321), AAV3.1/hu.9 (SEQ ID NO: 155 and 58 of WO2005033321), AAV3-11/rh.53 (SEQ ID NO: 186 and 176 of WO2005033321), AAV3-3 (SEQ ID NO: 200 of WO2005033321), AAV33.12/hu.17 (SEQ ID NO:4 of WO2005033321), AAV 33.4/hu.15 (SEQ ID NO: 50 of WO2005033321), AAV33.8/hu.16 (SEQ ID NO: 51 of WO2005033321), AAV3-9/rh.52 (SEQ ID NO: 96 and 18 of WO2005033321), AAV4-191rh.55 (SEQ ID NO: 117 of WO2005033321), AAV4-4 (SEQ ID NO: 201 and 218 of WO2005033321), AAV4-9/rh.54 (SEQ ID NO: 116 of WO2005033321), AAV5 (SEQ ID NO: 199 and 216 of WO2005033321), AAV52.1/hu.20 (SEQ ID NO: 63 of WO2005033321), AAV52/hu.19 (SEQ ID NO: 133 of WO2005033321), AAV5-22/rh.58 (SEQ ID No: 27 of WO2005033321), AAV5-3/rh.57 (SEQ ID NO: 105 of WO2005033321), AAV5-3/rh.57 (SEQ ID NO: 26 of WO2005033321), AAV58.2/hu.25 (SEQ ID NO: 49 of WO2005033321), AAV6 (SEQ ID NO: 203 and 220 of WO2005033321), AAV7 (SEQ ID NO: 222 and 213 of WO2005033321), AAV7,3/hu.7 (SEQ ID No: 55 of WO2005033321), AAV8 (SEQ ID NO: 223 and 214 of WO2005033321), AAVH-1/hu.1 (SEQ ID NO: 46 of WO2005033321), AAVH-5/hu.3 (SEQ ID NO: 44 of WO2005033321), AAVhu. (SEQ ID NO: 144 of WO2005033321), AAVhu.1.0 (SEQ ID NO: 156 of WO2005033321), AAVhu.11 (SEQ ID NO: 153 of WO2005033321), AAVhu.12 (WO2005033321 SEQ ID NO: 59), AAVhu.13 (SEQ ID NO: 129 of WO2005033321), AAVhu.14/AAV9 (SEQ ID NO: 123 and 3 of WO2005033321), AAVhu.15 (SEQ ID NO: 147 of WO2005033321), AAVhu.16 (SEQ ID NO: 148 of WO2005033321), AAVhu.17 (SEQ ID NO: 83 of WO2005033321), AAVhu.18 (SEQ NO: 149 of WO2005033321), AAVhu.19 (SEQ ID NO: 133 of WO2005033321), AAVhu.2 (SEQ ID NO: 143 of WO2005033321), AAVhu.20 (SEQ ID NO: 134 of WO2005033321), AAVhu.21 (SEQ ID NO: 135 of WO2005033321), AAVhu.22 (SEQ ID NO: 138 of WO2005033321), AAVhu.23.2 (SEQ ID NO: 137 of WO2005033321), AAVhu.24 (SEQ ID NO: 136 of WO2005033321), AAVhu.25 (SEQ ID NO: 146 of WO2005033321), AAVhu.27 (SEQ ID NO: 140 of WO2005033321), AAVhu.29 (SEQ ID NO: 132 of WO2005033321), AAVhu.3 (SEQ ID NO: 145 of WO2005033321), AAVhu.31 (SEQ ID NO: 121 of WO2005033321), AAVhu.32 (SEQ ID NO: 122 of WO2005033321), AAVhu.34 (SEQ ID NO: 125 of WO2005033321), AAVhu.35 (SEQ ID NO: 164 of WO2005033321), AAVhu.37 (SEQ ID NO: 88 of WO2005033321), AAVhu; 39 (SEQ ID NO: 102 of WO2005033321), AAVhu.4 (SEQ ID NO: 141 of WO2005033321), AAVhu.40 (SEQ ID NO: 87 of WO2005033321), AAVhu.41 (SEQ ID NO: 91 of WO2005033321), AAVhu.42 (SEQ ID NO: 85 of WO2005033321), AAVhu.43 (SEQ NO: 160 of WO2005033321), AAVhu.44 (SEQ ID NO: 144 of X/1102005033321), AAVhu.45 (SEQ ID NO: 127 of WO2005033321), AAVhu.46 (SEQ ID NO: 159 of WO2005033321), AAVhu.47 (SEQ ID NO: 128 of WO2005033321), AAVhu.48 (SEQ ID NO: 157 of WO2005033321), AAVhu.49 (SEQ ID NO: 189 of WO2005033321), AAVhu.51 (SEQ ID NO: 190 of WO2005033321), AAVhu.52 (SEQ ID NO: 191 of WO2005033321), AAVhu.53 (SEQ ID NO: 186 of WO2005033321), AAVhu.54 (SEQ ID NO: 188 of WO2005033321), AAVhu.55 (SEQ ID NO: 187 of WO2005033321), AAVhu.56 (SEQ ID NO: 192 of WO2005033321), AAVhu.57 (SEQ ID NO: 193 of WO2005033321), AAVhu.58 (SEQ ID NO: 194 of WO2005033321), AAVhu.6 (SEQ ID NO: 84 of WO2005033321), AAVhu.60 (SEQ ID NO: 184 of WO2005033321), AAVhu.61 (SEQ ID NO: 185 of WO2005033321), AAVhu.63 (SEQ ID NO: 195 of WO2005033321), AAVhu.64 (SEQ ID NO: 196 of WO2005033321), AAVhu.66 (SEQ ID NO: 197 of WO2005033321), AAVhu.67 (SEQ ID NO: 198 of WO2005033321), AAVhu.7 (SEQ ID NO: 150 of WO2005033321), AAVhu.8 (WO2005033321 SEQ ID NO: 12), AAVhu.9 (SEQ ID NO: 155 of WO2005033321), AAVLG-10/rh.40 (SEQ ID NO: 14 of WO2005033321), AAVLG-4/rh.38 (SEQ ID NO: 86 of WO2005033321), AAVLG-4/rh.38 (SEQ ID NO: 7 of WO2005033321), AAVN721-8/rh.43 (SEQ ID NO: 163 of WO2005033321), AAVN721-8/rh.43 (SEQ ID NO: 43 of WO2005033321), AAVpi.1 (WO2005033321 SEQ ID NO: 28), AAVpi.2 (WO2005033321 SEQ ID NO: 30), AAVpi.3 (WO2005033321 SEQ ID NO: 29), AAVrh.38 (SEQ ID NO: 86 of WO2005033321), AAVrh.40 (SEQ ID NO: 92 of WO2005033321), AAVrh.43 (SEQ ID NO: 163 of WO2005033321), AAVrh.44 (WO2005033321 SEQ ID NO: 34), AAVrh.45 (WO2005033321 SEQ ID NO: 41), AAVrh.47 (WO2005033321 SEQ ID NO: 38), AAVrh.48 (SEQ ID NO: 115 of WO2005033321), AAVrh.49 (SEQ ID NO: 103 of WO2005033321), AAVrh.50 (SEQ ID NO: 108 of WO2005033321), AAVrh.51 (SEQ ID NO: 104 of WO2005033321), AAVrh.52 (SEQ ID NO: 96 of WO2005033321), AAVrh.53 (SEQ ID NO: 97 of WO2005033321), AAVrh.55 (WO2005033321 SEQ ID NO: 37), AAVrh.56 (SEQ ID NO: 152 of WO2005033321), AAVrh.57 (SEQ ID NO: 105 of WO2005033321), AAVrh.58 (SEQ ID NO: 106 of WO2005033321), AAVrh.59 (WO2005033321 SEQ ID NO: 42), AAVrh.60 (WO2005033321 SEQ ID NO: 31), AAVrh.61 (SEQ ID NO: 107 of WO2005033321), AAVrh.62 (SEQ ID NO: 114 of WO2005033321), AAVrh.64 (SEQ ID NO: 99 of WO2005033321), AAVrh.65 (WO2005033321 SEQ ID NO: 35), AAVrh.68 (WO2005033321 SEQ ID NO: 16), AAVrh.69 (WO2005033321 SEQ ID NO: 39), AAVrh.70 (WO2005033321 SEQ ID NO: 20), AAVrh.72 (WO2005033321 SEQ ID NO: 9), or variants thereof including, but not limited to, AAVcy.2, AAVcy.3, AAVcy.4, AAVcy.5, AAVcy.6, AAVrh.12, AAVrh.17, AAVrh.18, AAVrh.19, AAVrh.21, AAVrh.22, AAVrh.23, AAVrh.24, AAVrh.25, AAVrh.25/42 15, AAVrh.31, AAVrh.32, AAVrh.33, AAVrh.34, AAVrh.35, AAVrh.36, AAVrh.37, AAVrh14. Non limiting examples of variants include SEQ ID NO: 13, 15, 17, 19, 24, 36, 40, 45, 47, 48, 51-54, 60-62, 64-77, 79, 80, 82, 89, 90, 93-95, 98, 100, 101, 109-113, 118-120, 124, 126, 131, 139, 142, 151,154, 158, 161, 162, 165-183, 202, 204-212, 215, 219, 224-236, of WO2005033321, the contents of which are herein incorporated by reference in their entirety.

In some embodiments, the AAV serotype may be, or have, a sequence as described in International Publication No. WO2015168666, the contents of which are herein incorporated by reference in their entirety, such as, but not limited to, AAVrh8R (SEQ ID NO: 9 of WO2015168666), AAVrh8R A586R mutant (SEQ ID NO: 10 of WO2015168666), AAVrh8R R533A mutant (SEQ ID NO: 11 of WO2015168666), or variants thereof.

In some embodiments, the AAV serotype may be, or have, a sequence as described in U.S. Pat. No. 9,233,131, the contents of which are herein incorporated by reference in their entirety, such as, but not limited to, AAVhE1.1 (SEQ ID NO:44 of U.S. Pat. No. 9,233,131), AAVhEr1.5 (SEQ. ID NO:45 of US9233131), AAVhER1,14 (SEQ NO:46 of US9233131), AAVhEr1.8 (SEQ ID NO:47 of U.S. Pat. No. 9,233,131), AAVhEr1.16 (SEQ ID NO:48 of U.S. Pat. No. 9,233,131), AAVhEr1.18 (SEQ ID NO:49 of U.S. Pat. No. 9,233,131), AAVhEr1.35 (SEQ ID NO:50 of U.S. Pat. No. 9,233,131), AAVhEr1.7 (SEQ ID NO:51 of U.S. Pat. No. 9,233,131), AAVhEr1.36 (SEQ ID NO:52 of US9233131), AAVhEr2,29 (SEQ ID NO:53 of US9233131), AAVhEr2,4 (SEQ ID NO:54 of US9233131), AAVhEr2.16 (SEQ ID NO:55 of U.S. Pat. No. 9,233,131), AAVhEr2.30 (SEQ ID NO:56 of U.S. Pat. No. 9,233,131), AAVhEr2.31 (SEQ ID NO:58 of U.S. Pat. No. 9,233,131), AAVhEr2.36 (SEQ ID NO:57 of US9233131), AAVhER1.23 (SEQ NO:53 of US9233131), AAVhEr3.1 (SEQ NO:59 of U.S. Pat. No. 9,233,131), AAV2.5T (SEQ ID NO:42 of US9233131), or variants thereof. 1:00801 In some embodiments, the AAV serotype may be, or have, a sequence as described in United States Patent Publication No. US20150376607, the contents of which are herein incorporated by reference in their entirety, such as, but not limited to, AAV-PAEC (SEQ ID NO:1 of US20150376607), AAV-LK01 (SEQ NO:2 of US20150376607), AAV-LK02 (SEQ ID NO:3 of US20150376607), AAV-LK03 (SEQ NO:4 of US20150376607), AAV-LK04 (SEQ ID NO:5 of US20150376607), AAV-LK05 (SEQ ID NO:6 of US20150376607), AAV-LK06 (SEQ ID NO:7 of US20150376607), AAV-LK07 (SEQ ID NO:8 of US20150376607), AAV-1,108 (SEQ 11) NO:9 of US20150376607), AAV-LK09 (SEQ ID NO:10 of US20150376607), AAV-LK10 (SEQ ID NO:11 of US20150376607), AAV-LK11 (SEQ ID NO:12 of US20150376607), AAV-LK 12 (SEQ ID NO:13 of US20150376607), AAV-LK13 (SEQ ID NO:14 of US20150376607), AAV-LK14 (SEQ ID NO:15 of US20150376607), AAV-LK 15 (SEQ NO:16 of US20150376607), AAV-LK16 (SEQ ID NO:17 of US20150376607), AAV-LK17 (SEQ ID NO:18 of US20150376607), AAV-LK18 (SEQ ID NO:19 of US20150376607), AAV-LK19 (SEQ ID NO:20 of US20150376607), AAV-PAEC2 (SEQ ID NO:21 of US20150376607), AAV-PAEC24 (SEQ ID NO:22 of US20150376607), AAV-PAEC6 (SEQ NO:23 of US20150376607), AAV-PAEC7 (SEQ ID NO:24 of US20150376607), AAV-PAEC8 (SEQ ID NO:25 of US20150376607), AAV-PAEC11 (SEQ NO:26 of US20150376607), AAV-PAEC12 (SEQ ID NO:27, of US20150376607), or variants thereof.

In some embodiments, the AAV serotype may be, or have, a sequence as described in U.S. Pat. No. 9,163,261, the contents of which are herein incorporated by reference in their entirety, such as, but not limited to, AAV-2-pre-miRNA-101 (SEQ NO: 1 U.S. Pat. No. 9,163,261), or variants thereof.

In some embodiments, the AAV serotype may be, or have, a sequence as described in United States Patent Publication No. 20150376240, the contents of which are herein incorporated by reference in their entirety, such as, but not limited to, AAV-8h (SEQ ID NO: 6 of US20150376240), AAV-8b (SEQ ID NO: 5 of US20150376240), AAV-h (SEQ ID NO: 2 of US20150376240), AAV-b (SEQ ID NO: 1 of US20150376240), or variants thereof

In some embodiments, the AAV serotype may be, or have, a sequence as described in United States Patent Publication No. US20160017295, the contents of which are herein incorporated by reference in their entirety, such as, but not limited to, AAV SM 10-2 (SEQ ID NO: 22 of US20160017295), AAV Shuffle 1001 (SEQ ID NO: 23 of US20160017295), AAV Shuffle 100-3 (SEQ ID NO: 24 of US20160017295), AAV Shuffle 100-7 (SEQ ID NO: 25 of US20160017295), AAV Shuffle 10-2 (SEQ ID NO: 34 of US20160017295), AAV Shuffle 10-6 (SEQ ID NO: 35 of US20160017295), AAV Shuffle 10-8 (SEQ ID NO: 36 of US20160017295), AAV Shuffle 100-2 (SEQ ID NO: 37 of US20160017295), AAV SM 10-1 (SEQ ID NO: 38 of US20160017295), AAV SM 10-8 (SEQ ID NO: 39 of US20160017295), AAV SM 100-3 (SEQ ID NO: 40 of US20160017295) AAV SM 100-10 (SEQ ID NO: 41 of US20160017295), or variants thereof.

In some embodiments, the AAV serotype may be, or have, a sequence as described in United States Patent Publication No. US20150238550, the contents of which are herein incorporated by reference in their entirety, such as, but not limited to, BNP61, AAV (SEQ ID NO: 1 of US20150238550), BNP62, AAV (SEQ ID NO: 3 of US20150238550), BNP63, AAV (SEQ ID NO: 4 of US20150238550), or variants thereof.

In some embodiments, the AAV serotype may be or may have a sequence as described in United States Patent Publication No. US20150315612, the contents of which are herein incorporated by reference in their entirety, such as, but not limited to, AAVrh.50 (SEQ ID NO: 108 of US20150315612), AAVrh.43 (SEQ ID NO: 163 of US20150315612), AAVrh.62 (SEQ ID NO: 114 of US20150315612), AAVrh.48 (SEQ ID NO: 115 of US20150315612), AAVhu.19 (SEQ ID NO: 133 of U.S. Pat. No. 2,015,031.5612), AAVhu.11 (SEQ ID NO: 153 of US20150315612), AAVhu.53 (SEQ ID NO: 186 of US20150315612), AAV4-8/rh.64 (SEQ ID NO: 15 of US20150315612), AAVLG-9/hu.39 (SEQ ID NO: 24 of US20150315612), AAV54.5/hu.23 (SEQ ID NO: 60 of US20150315612), AAV54.2/hu.22 (SEQ ID NO: 67 of US20150315612), AAV54,7/hu.24 (SEQ ID NO: 66 of US20150315612), AAV54.1/hu.21 (SEQ ID No: 65 of US20150315612), AAV54.4R/hu.27 (SEQ ID NO: 64 of US20150315612), AAV46.2/hu.28 (SEQ ID NO: 68 of US20150315612), AAV46.6/hu.29 (SEQ ID No: 69 of US20150315612), AAV128.1/hu.43 (SEQ ID NO: 80 of US20150315612), or variants thereof.

In some embodiments, the AAV serotype may be, or have, a sequence as described in International Publication No. WO2015121501, the contents of which are herein incorporated by reference in their entirety, such as, but not limited to, true type AAV (ttAAV) (SEQ ID NO: 2 of WO2015121501), “UPenn AAV10” (SEQ ID NO: 8 of WO2015121501), “Japanese AAV10” (SEQ ID NO: 9 of WO2015121501), or variants thereof.

According to the present disclosure, AAT capsid serotype selection or use may be from a variety of species. In some embodiments, the AAV may be an avian AAV (AAAV). The AAAV serotype may be, or have, a sequence as described in U.S. Pat. No. 9,238,800, the contents of which are herein incorporated by reference in their entirety, such as, but not limited to, AAAV (SEQ ID NO: 1, 2, 4, 6, 8, 10, 12, and 14 of U.S. Pat. No. 9,238,800), or variants thereof.

In some embodiments, the AAV may be a bovine AAV (BAAV). The BAAV serotype may be, or have, a sequence as described in U.S. Pat. No. 9,193,769, the contents of which are herein incorporated by reference in their entirety, such as, but not limited to, BAAV (SEQ ID NO: 1 and 6 of U.S. Pat. No. 9,193,769), or variants thereof. The BAAV serotype may be or have a sequence as described in U.S. Pat. No. 7,427,396, the contents of which are herein incorporated by reference in their entirety, such as, but not limited to, BAAV (SEQ ID NO: 5 and 6 of US7427396), or variants thereof.

In some embodiments, the AAV may be a caprine AAV. The caprine AAV serotype may be, or have, a sequence as described in U.S. Pat. No. 7,427,396, the contents of which are herein incorporated by reference in their entirety, such as, but not limited to, caprine AAV (SEQ ID NO: 3 of US7427396), or variants thereof.

In other embodiments the AAV may be engineered as a hybrid AAV from two or more parental serotypes. In some embodiments, the AAV may be AAV2G9 which comprises sequences from AAV2 and AAV9. The AAV2G9, AAV serotype may be, or have, a sequence as described in United States Patent Publication No. US20160017005, the contents of which are herein incorporated by reference in its entirety.

In some embodiments, the AAV may be a serotype generated by the AAV9 capsid library with mutations in amino acids 390-627 (VP1 numbering) as described by Pulicherla et al. (Molecular Therapy 19(6):1070-1078 (2011), the contents of which are herein incorporated by reference in their entirety. The serotype and corresponding nucleotide and amino acid substitutions may be, but is not limited to, AAV9.1 (G1594C; D53211), AAV6.2 (T1418A and T1436X; V4731) and 1479K), AAV9.3 (T1238A; F413Y), AAV9.4 (T1250C and A1617T; F4175), AAV9.5 (A1235G, A1314T, A1642G, C1760T; Q4121T, T548A, A587V), AAV9.6 (T1231A; F411I), AAV9.9 (G1203A, G1785T; W595C), AAV9.10 (A1.500G, T1.676C; M559T), AAV9.11. (A1425T, A1702C, A1769I; T5681), Q590 L), AAV9.13 (A1369C, A1720T; N457H, T574S), AAV9.14 (T1340A, T1362C, T1560C, G1713A; 11,447E1) AAV9.16 (A1775T; Q5921), AVV9.24 (T1507c, T1521G; W503R), AAV9.26 (A1337G, A1769C; Y446C, Q590P), AAV9.33 (A1667C; D556A), AAV9.34 (A1534G, C17941; N512D), AAV9.35 (A12891, 11450A, C14941, A15151, C1794A, G1816A; 04301, Y484N, N98K, V6061), AAV9,40 (A16941, E565V), AAA/9.41 (A13481, 11362C, 1450S), AAV9.44 (A1684C, A1701T, A1737G; N56211, K567N), AAV9.45 (A14921, C18041; N498Y, L602F), AVV9.46 (G1441C, T1525C, T1549G; G481R, W509R, L517V), 9.47 (G1241A, G1358A, A1669G, C17451; 5414N, G453D, K557E, T5821), AAV9.48 (C14451, A17361; P482L, Q579L), AAV9.50 (A16381, C16831, 171805A; 054613, L60211), AAV9.53 (s1301A, A1405C, C16641, G1811T, R134Q, S469R, A555V, G6041/), AAV9.54 (C1531A, T1609A; L5111, L537M), AAV9.55 (T1605A; F535L), AAV9.58 (C1475T, C1579A, T4921, H527N), AAV59 (11336C; Y446H), AAV9.61 (A14931; N4981), AAV9.64 (C1531A, A16171; L5111), AAV9.65 (C1335T, T1530C, C568A; A523D), AAV9.68 (C1510A; P5041), AAV9.80 (G1441A; G481R), AAV9.83 (C1402A, A1500T; P4681, E500D), AAV9.87 (T1464C; T1468C; 5490P), AAV9.90 (A1196T, Y399F), AAV9.91 (T1316G, A1583T, C1782G, T1806C; L439R, K528I), AAV9.93 (A1273G, A1421G, A1638C, C1712T, G1732A, A17441, A18321; S425G, Q4748, Q546H, P571L, G578R, 1582S D611V), AAV9.94 (A16751; M559L) and AAV995 (11605A; F535L).

In some embodiments, the AAV serotype may be, or have, a sequence as described in International Publication No. WO2016049230, the contents of which are herein incorporated by reference in their entirety, such as, but not limited to AAVF1/HSC1 (SEQ ID NO: 2 and 20 of WO2016049230), AAVF2/HSC2 (SEQ ID NO: 3 and 21 of WO2016049230), AAVF3/HSC3 (SEQ ID NO: 5 and 22 of WO2016049230), AAVF4/HSC4 (SEQ ID NO: 6 and 23 of WO2016049230), AAVF5/HSC5 (SEQ ID NO: 11 and 25 of WO2016049230), AAVF6/HSC6 (SEQ ID NO: 7 and 24 of WO2016049230), AAVF7/HSC7 (SEQ ID NO: 8 and 27 of WO2016049230), AAVF8/HSC8 (SEQ ID NO: 9 and 28 of WO2016049230), AAVF9/HSC9 (SEQ ID NO: 10 and 29 of WO2016049230), AAVF11/HSC11 (SEQ ID NO: 4 and 26 of WO2016049230), AAVF12/HSC12 (SEQ ID NO: 12 and 30 of WO2016049230), AAVF13/HSC13 (SEQ ID NO: 14 and 31 of WO2016049230), AAVF14/HSC14 (SEQ ID NO: 15 and 32 of WO2016049230), AAVF15/HSC15 (SEQ ID NO: 16 and 33 of W(012016049230), AAVF16/HSC16 (SEQ ID NO: 17 and 34 of WO2016049230), AAVF17/HSC17 (SEQ ID NO: 13 and 35 of WO2016049230), or variants or derivatives thereof.

In some embodiments, the AAV serotype may be, or have, a sequence as described in U.S. Pat. No. 8,734,809, the contents of which are herein incorporated by reference in their entirety, such as, but not limited to, AAV CBr-E1 (SEQ ID NO: 13 and 87 of U.S. Pat. No. 8,734,809), AAV CBr-E2 (SEQ ID NO: 14 and 88 of US8734809), AAV CBr-E3 (SEQ ID NO: 15 and 89 of U.S. Pat. No. 8,734,809), AAV CBr-E4 (SEQ ID NO: 16 and 90 of U.S. Pat. No. 8,734,809), AAV CBr-E5 (SEQ ID NO: 17 and 91 of U.S. Pat. No. 8,734,809), AAV CBr-e5 (SEQ ID NO: 18 and 92 of US8734809), AAV CBr-E6 (SEQ ID NO: 19 and 93 of U.S. Pat. No. 8,734,809), AAV CBr-E7 (SEQ ID NO: 20 and 94 of U.S. Pat. No. 8,734,809), AAV CBr-E8 (SEQ ID NO: 21 and 95 of U.S. Pat. No. 8,734,809), AAV (SEQ ID NO: 22 and 96 of US8734809), AAV CIA-D2 (SEQ ID NO: 23 and 97 of U.S. Pat. No. 8,734,809), AAV CLv-D3 (SEQ ID NO: 24 and 98 of U.S. Pat. No. 8,734,809), AAV CLv-D4 (SEQ ID NO: 25 and 99 of U.S. Pat. No. 8,734,809), AAV CIA-D5 (SEQ ID NO: 26 and 100 of US8734809), AAV CIA-D6 (SEQ ID NO: 27 and 101 of U.S. Pat. No. 8,734,809), AAV CLv-D7 (SEQ ID NO: 28 and 102 of US8734809), AAV CIA-D8 (SEQ ID NO: 29 and 103 of U.S. Pat. No. 8,734,809), AAV CLv-E1 (SEQ ID NO: 13 and 87 of U.S. Pat. No. 8,734,809), AAV CIA-R1 (SEQ ID NO: 30 and 104 of U.S. Pat. No. 8,734,809), AAV CLv-R2 (SEQ ID NO: 31 and 105 of U.S. Pat. No. 8,734,809), AAV CIA-R3 (SEQ ID NO: 32 and 106 of US8734809), AAV CLv-R4 (SEQ ID NO: 33 and 107 of U.S. Pat. No. 8,734,809), AAV CLv-R5 (SEQ ID NO: 34 and 108 of U.S. Pat. No. 8,734,809), AAV CLv-R6 (SEQ ID NO: 35 and 109 of U.S. Pat. No. 8,734,809), AAV CLv-R7 (SEQ ID NO: 36 and 110 of U.S. Pat. No. 8,734,809), AAV CLv-R8 (SEQ ID NO: X and X of U.S. Pat. No. 8,734,809), AAV CLv-R9 (SEQ ID NO: X and X of US8734809), AAV CLg-F1 (SEQ ID NO: 39 and 113 of U.S. Pat. No. 8,734,809), AAV CLg-F2 (SEQ ID NO: 40 and 114 of U.S. Pat. No. 8,734,809), AAV CLg-F3 (SEQ ID NO: 41 and 115 of US8734809), AAV CLg-F4 (SEQ ID NO: 42 and 116 of U.S. Pat. No. 8,734,809), AAV CLg-F5 (SEQ ID NO: 43 and 117 of US8734809), AAV CLg-F6 (SEQ ID NO: 43 and 117 of US8734809), AAS CLg-F7 (SEQ ID NO: 44 and 118 of U.S. Pat. No. 8,734,809), AAV CLg-F8 (SEQ ID NO: 43 and 117 of U.S. Pat. No. 8,734,809), AAV CSp-1 (SEQ ID NO: 45 and 119 of US8734809), AAV CSp-10 (SEQ ID NO: 46 and 120 of U.S. Pat. No. 8,734,809), AAS CSp-11 (SEQ ID NO: 47 and 121 of U.S. Pat. No. 8,734,809), AAV CSp-2 (SEQ ID NO: 48 and 122 of US8734809), AAV CSp-3 (SEQ ID NO: 49 and 123 of U.S. Pat. No. 8,734,809), AAV CSp-4 (SEQ ID NO: 50 and 124 of U.S. Pat. No. 8,734,809), AAV CSp-6 (SEQ ID NO: 51 and 125 of US8734809), AAV CSp-7 (SEQ ID NO: 52 and 126 of U.S. Pat. No. 8,734,809), AAV CSp-8 (SEQ ID NO: 53 and 127 of US8734809), AAT CSp-9 (SEQ ID NO: 54 and 128 of U.S. Pat. No. 8,734,809), AAV CHt-2 (SEQ ID NO: 55 and 129 of U.S. Pat. No. 8,734,809), AAV CHt-3 (SEQ ID NO: 56 and 130 of U.S. Pat. No. 8,734,809), AAV CKd-1 (SEQ ID NO: 57 and 131 of U.S. Pat. No. 8,734,809), AAV CKd-10 (SEQ ID NO: 58 and 132 of U.S. Pat. No. 8,734,809), AAV CKd-2 (SEQ ID NO: 59 and 133 of US8734809), AAV CKd-3 (SEQ ID NO: 60 and 134 of US8734809), AAV CKd-4 (SEQ ID NO: 61 and 135 of U.S. Pat. No. 8,734,809), AAV CKd-6 (SEQ ID NO: 62 and 136 of U.S. Pat. No. 8,734,809), AAV CKd-7 (SEQ ID NO: 63 and 137 of US8734809), AAV CKd-8 (SEQ ID NO: 64 and 138 of U.S. Pat. No. 8,734,809), AAV CLv-1 (SEQ ID NO: 35 and 139 of U.S. Pat. No. 8,734,809), AAV CLv-12 (SEQ ID NO: 66 and 140 of U.S. Pat. No. 8,734,809), AAV CLv-13 (SEQ ID NO: 67 and 141 of U.S. Pat. No. 8,734,809), AAV CLv-2 (SEQ ID NO: 68 and 142 of U.S. Pat. No. 8,734,809). AAV CLv-3 (SEQ ID NO: 69 and 143 of U.S. Pat. No. 8,734,809), AAV (SEQ ID NO: 70 and 144 of U.S. Pat. No. 8,734,809), AAV CLv-6 (SEQ ID NO: 71 and 145 of U.S. Pat. No. 8,734,809), AAV CLv-8 (SEQ ID NO: 72 and 146 of U.S. Pat. No. 8,734,809), AAV CI(d-B1 (SEQ ID NO: 73 and 147 of U.S. Pat. No. 8,734,809), AAV CM-B2 (SEQ ID NO: 74 and 148 of U.S. Pat. No. 8,734,809), AAV C1(d-B3 (SEQ ID NO: 75 and 149 of U.S. Pat. No. 8,734,809), AAV CKd-B4 (SEQ ID NO: 76 and 150 of US8734809), AAV CKd-135 (SEQ ID NO: 77 and 151 of U.S. Pat. No. 8,734,809), AAV CM-Bo (SEQ ID NO: 78 and 152 of U.S. Pat. No. 8,734,809), AAV CKd-B7 (SEQ ID NO: 79 and 153 of U.S. Pat. No. 8,734,809), AAV CKd-B8 (SEQ ID NO: 80 and 154 of U.S. Pat. No. 8,734,809), AAV CKd-H1 (SEQ ID NO: 81 and 155 of US8734809), AAV CKd-H2 (SEQ ID NO: 82 and 156 of U.S. Pat. No. 8,734,809), AAV CKd-H3 (SEQ ID NO: 83 and 157 of U.S. Pat. No. 8,734,809), AAV CKd-H4 (SEQ ID NO: 84 and 158 of U.S. Pat. No. 8,734,809), AAV CKd-H5 (SEQ ID NO: 85 and 159 of U.S. Pat. No. 8,734,809), AAV CKd-H6 (SEQ ID NO: 77 and 151 of U.S. Pat. No. 8,734,809), AAV CHt-1 (SEQ ID NO: 86 and 160 of US8734809), AAV (SEQ ID NO: 171 of U.S. Pat. No. 8,734,809), AAV CLv1-2 (SEQ ID NO: 172 of U.S. Pat. No. 8,734,809), AAV CLv1-3 (SEQ ID NO: 173 of U.S. Pat. No. 8,734,809), AAV CLv1-4 (SEQ ID NO: 174 of U.S. Pat. No. 8,734,809), AAV Clv1-7 (SEQ ID NO: 175 of U.S. Pat. No. 8,734,809), AAV Clv1-8 (SEQ ID NO: 176 of US8734809), AAV Clv1-9 (SEQ ID NO: 177 of US8734809), AAV Clv1-10 (SEQ ID NO: 178 of U.S. Pat. No. 8,734,809), AAV.VR-355 (SEQ ID NO: 181 of U.S. Pat. No. 8,734,809), AAV.hu.48R3 (SEQ ID NO: 183 of U.S. Pat. No. 8,734,809), or variants or derivatives thereof.

In some embodiments, the AAV serotype may be, or have, a sequence as described in International Publication No. WO2016065001, the contents of which are herein incorporated by reference in their entirety, such as, but not limited to AAV CHt-P2 (SEQ ID NO: 1 and 51 of WO2016065001), AAV CHt-P5 (SEQ ID NO: 2 and 52 of WO2016065001), AAV CHt-P9 (SEQ ID NO: 3 and 53 of WO2016065001), AAV CBr-7.1 (SEQ ID NO: 4 and 54 of WO2016065001), AAV CBr-7.2 (SEQ ID NO: 5 and 55 of WO2016065001), AAV CBr-7.3 (SEQ ID NO: 6 and 56 of WO2016065001), AAV CBr-7.4 (SEQ ID NO: 7 and 57 of WO2016065001), AAV CBr-7.5 (SEQ ID NO: 8 and 58 of WO2016065001), AAV CBr-7.7 (SEQ ID NO: 9 and 59 of WO2016065001), AAV CBr-7.8 (SEQ ID NO: 10 and 60 of WO2016065001), AAV CBr-7,10 (SEQ ID NO: 11 and 61 of WO2016065001), AAV CKd-N3 (SEQ ID NO: 12 and 62 of WO2016065001), AAV CKd-N4 (SEQ ID NO: 13 and 63 of WO2016065001), AAV CKd-N9 (SEQ ID NO: 14 and 64 of WO2016065001), AAV CLv-L4 (SEQ ID NO: 15 and 65 of WO2016065001), AAV CLv-L5 (SEQ ID NO: 16 and 66 of WO2016065001), AAV CLv-L6 (SEQ ID NO: 17 and 67 of WO2016065001), AAV CLv-K1 (SEQ ID NO: 18 and 68 of WO2016065001), AAV CLv-K3 (SEQ ID NO: 19 and 69 of WO2016065001), AAV CLv-K6 (SEQ ID NO: 20 and 70 of WO2016065001), AAV CLv-M1 (SEQ ID NO: 21 and 71 of WO2016065001), AAV CLv-M11 (SEQ ID NO: 22 and 72 of WO2016065001), AAV CLv-M2 (SEQ ID NO: 23 and 73 of WO2016065001), AAV CLv-M5 (SEQ ID NO: 24 and 74 of WO2016065001), AAV CLv-M6 (SEQ ID NO: 25 and 75 of WO2016065001), AAV CLv-M7 (SEQ ID NO: 26 and 76 of WO2016065001), AAV CLv-M8 (SEQ ID NO: 27 and 77 of WO2016065001), AAV CLv-M9 (SEQ ID NO: 28 and 78 of WO2016065001), AAV Mt-Pi (SEQ ID NO: 29 and 79 of WO2016065001), AAV CHt-P6 (SEQ ID NO: 30 and 80 of WO2016065001), AAV CHt-P8 (SEQ ID NO: 31 and 81 of WO2016065001), AAV CHt-6.1 (SEQ ID NO: 32 and 82 of WO2016065001), AAV CHI-6.10 (SEQ ID NO: 33 and 83 of WO2016065001), AAV CHt-6.5 (SEQ ID NO: 34 and 84 of WO2016065001), AAV CHt-6.6 (SEQ ID NO: 35 and 85 of WO2016065001), AAV CHt-6.7 (SEQ ID NO: 36 and 86 of WO2016065001), AAV CHt-6.8 (SEQ ID NO: 37 and 87 of WO2016065001), AAV CSp-8.10 (SEQ ID NO: 38 and 88 of WO2016065001), AAV CSp-8,2 (SEQ ID NO: 39 and 89 of WO2016065001), AAV CSp-8.4 (SEQ ID NO: 40 and 90 of WO2016065001), AAV CSp-8.5 (SEQ ID NO: 41 and 91 of WO2016065001), AAV CSp-8.6 (SEQ ID NO: 42 and 92 of WO2016065001), AAV CSp-8.7 (SEQ ID NO: 43 and 93 of WO2016065001), AAV CSp-8.8 (SEQ ID NO: 44 and 94 of WO2016065001), AAV CSp-8.9 (SEQ ID NO: 45 and 95 of WO2016065001), AAV CBr-B7.3 (SEQ ID NO: 46 and 96 of WO2016065001), AAV CBr-B7,4 (SEQ ID NO: 47 and 97 of WO2016065001), AAV3B (SEQ ID NO: 48 and 98 of WO2016065001), AAV4 (SEQ ID NO: 49 and 99 of WO2016065001), AAV5 (SEQ ID NO: 50 and 100 of WO2016065001), or variants or derivatives thereof.

In some embodiments, the AAV may be a serotype selected from any of those found in Table 1.

In some embodiments, the AAV serotype may comprise a sequence, fragment or variant thereof, of the sequences in Table 1.

In some embodiments, the AAV serotype may be encoded by a sequence, fragment or variant as described in Table 1.

In some embodiments, the AAV serotype may comprise a sequence given by any of SEQ ID NO: 1-1723.

In some embodiments, the AAV serotype may be encoded by a sequence given by any of SEQ ID NO: 1-1723.

TABLE 1
AAV Serotypes

| Serotype | SEQ ID NO | Reference Information |
|---|---|---|
| VOY101 | 1 and 1722 | — |
| VOY201 | 1723 | — |
| PHP.N/PHP.B-DGT | 2 | WO2017100671 SEQ ID NO: 46 |
| AAVPHP.B or G2B-26 | 3 | WO2015038958 SEQ ID NO: 8 and 13 |
| AAVPHP.B | 4 | WO2015038958 SEQ ID NO: 9 |
| AAVG2B-13 | 5 | WO2015038958 SEQ ID NO: 12 |
| AAVTH1.1-32 | 6 | WO2015038958 SEQ ID NO: 14 |
| AAVTH1.1-35 | 7 | WO2015038958 SEQ ID NO: 15 |
| PHP.S/G2A12 | 8 | WO2017100671 SEQ ID NO: 47 |
| AAV9/hu.14 K449R | 9 | WO2017100671 SEQ ID NO: 45 |
| AAV1 | 10 | US20150159173 SEQ ID NO: 11, US20150315612 SEQ |
| | | ID NO: 202 |
| AAV1 | 11 | US20160017295 SEQ ID NO: 1, US20030138772 SEQ |
| | | ID NO: 64, US20150159173 SEQ ID NO: 27, |
| | | US20150315612 SEQ ID NO: 219, U.S. Pat. No. 7,198,951 |
| | | SEQ ID NO: 5 |
| AAV1 | 12 | US20030138772 SEQ ID NO: 6 |
| AAV1.3 | 13 | US20030138772 SEQ ID NO: 14 |
| AAV10 | 14 | US20030138772 SEQ ID NO: 117 |
| AAV10 | 15 | WO2015121501 SEQ ID NO: 9 |
| AAV10 | 16 | WO2015121501 SEQ ID NO: 8 |
| AAV11 | 17 | US20030138772 SEQ ID NO: 118 |
| AAV12 | 18 | US20030138772 SEQ ID NO: 119 |
| AAV2 | 19 | US20150159173 SEQ ID NO: 7, US20150315612 SEQ |
| | | ID NO: 211 |
| AAV2 | 20 | US20030138772 SEQ ID NO: 70, US20150159173 SEQ |
| | | ID NO: 23, US20150315612 SEQ ID NO: 221, |
| | | US20160017295 SEQ ID NO: 2, U.S. Pat. No. 6,156,303 |
| | | SEQ ID NO: 4, U.S. Pat. No. 7,198,951 SEQ ID NO: 4, |
| | | WO2015121501 SEQ ID NO: 1 |
| AAV2 | 21 | U.S. Pat. No. 6,156,303 SEQ ID NO: 8 |
| AAV2 | 22 | US20030138772 SEQ ID NO: 7 |
| AAV2 | 23 | U.S. Pat. No. 6,156,303 SEQ ID NO: 3 |
| AAV2.5T | 24 | U.S. Pat. No. 9,233,131 SEQ ID NO: 42 |
| AAV223.10 | 25 | US20030138772 SEQ ID NO: 75 |
| AAV223.2 | 26 | US20030138772 SEQ ID NO: 49 |
| AAV223.2 | 27 | US20030138772 SEQ ID NO: 76 |
| AAV223.4 | 28 | US20030138772 SEQ ID NO: 50 |
| AAV223.4 | 29 | US20030138772 SEQ ID NO: 73 |
| AAV223.5 | 30 | US20030138772 SEQ ID NO: 51 |
| AAV223.5 | 31 | US20030138772 SEQ ID NO: 74 |
| AAV223.6 | 32 | US20030138772 SEQ ID NO: 52 |
| AAV223.6 | 33 | US20030138772 SEQ ID NO: 78 |
| AAV223.7 | 34 | US20030138772 SEQ ID NO: 53 |
| AAV223.7 | 35 | US20030138772 SEQ ID NO: 77 |
| AAV29.3 | 36 | US20030138772 SEQ ID NO: 82 |
| AAV29.4 | 37 | US20030138772 SEQ ID NO: 12 |
| AAV29.5 | 38 | US20030138772 SEQ ID NO: 83 |
| AAV29.5 (AAVbb.2) | 39 | US20030138772 SEQ ID NO: 13 |
| AAV3 | 40 | US20150159173 SEQ ID NO: 12 |
| AAV3 | 41 | US20030138772 SEQ ID NO: 71, US20150159173 SEQ |
| | | ID NO: 28, US20160017295 SEQ ID NO: 3, |
| | | U.S. Pat. No. 7,198,951 SEQ ID NO: 6 |
| AAV3 | 42 | US20030138772 SEQ ID NO: 8 |
| AAV3.3b | 43 | US20030138772 SEQ ID NO: 72 |
| AAV3-3 | 44 | US20150315612 SEQ ID NO: 200 |
| AAV3-3 | 45 | US20150315612 SEQ ID NO: 217 |
| AAV3a | 46 | U.S. Pat. No. 6,156,303 SEQ ID NO: 5 |
| AAV3a | 47 | U.S. Pat. No. 6,156,303 SEQ ID NO: 9 |
| AAV3b | 48 | U.S. Pat. No. 6,156,303 SEQ ID NO: 6 |
| AAV3b | 49 | U.S. Pat. No. 6,156,303 SEQ ID NO: 10 |
| AAV3b | 50 | U.S. Pat. No. 6,156,303 SEQ ID NO: 1 |
| AAV4 | 51 | US20140348794 SEQ ID NO: 17 |
| AAV4 | 52 | US20140348794 SEQ ID NO: 5 |
| AAV4 | 53 | US20140348794 SEQ ID NO: 3 |
| AAV4 | 54 | US20140348794 SEQ ID NO: 14 |
| AAV4 | 55 | US20140348794 SEQ ID NO: 15 |
| AAV4 | 56 | US20140348794 SEQ ID NO: 19 |
| AAV4 | 57 | US20140348794 SEQ ID NO: 12 |
| AAV4 | 58 | US20140348794 SEQ ID NO: 13 |
| AAV4 | 59 | US20140348794 SEQ ID NO: 7 |
| AAV4 | 60 | US20140348794 SEQ ID NO: 8 |
| AAV4 | 61 | US20140348794 SEQ ID NO: 9 |
| AAV4 | 62 | US20140348794 SEQ ID NO: 2 |
| AAV4 | 63 | US20140348794 SEQ ID NO: 10 |
| AAV4 | 64 | US20140348794 SEQ ID NO: 11 |
| AAV4 | 65 | US20140348794 SEQ ID NO: 18 |
| AAV4 | 66 | US20030138772 SEQ ID NO: 63, US20160017295 SEQ |
| | | ID NO: 4, US20140348794 SEQ ID NO: 4 |
| AAV4 | 67 | US20140348794 SEQ ID NO: 16 |
| AAV4 | 68 | US20140348794 SEQ ID NO: 20 |
| AAV4 | 69 | US20140348794 SEQ ID NO: 6 |
| AAV4 | 70 | US20140348794 SEQ ID NO: 1 |
| AAV42.2 | 71 | US20030138772 SEQ ID NO: 9 |
| AAV42.2 | 72 | US20030138772 SEQ ID NO: 102 |
| AAV42.3b | 73 | US20030138772 SEQ ID NO: 36 |
| AAV42.3B | 74 | US20030138772 SEQ ID NO: 107 |
| AAV42.4 | 75 | US20030138772 SEQ ID NO: 33 |
| AAV42.4 | 76 | US20030138772 SEQ ID NO: 88 |
| AAV42.8 | 77 | US20030138772 SEQ ID NO: 27 |
| AAV42.8 | 78 | US20030138772 SEQ ID NO: 85 |
| AAV43.1 | 79 | US20030138772 SEQ ID NO: 39 |
| AAV43.1 | 80 | US20030138772 SEQ ID NO: 92 |
| AAV43.12 | 81 | US20030138772 SEQ ID NO: 41 |
| AAV43.12 | 82 | US20030138772 SEQ ID NO: 93 |
| AAV43.20 | 83 | US20030138772 SEQ ID NO: 42 |
| AAV43.20 | 84 | US20030138772 SEQ ID NO: 99 |
| AAV43.21 | 85 | US20030138772 SEQ ID NO: 43 |
| AAV43.21 | 86 | US20030138772 SEQ ID NO: 96 |
| AAV43.23 | 87 | US20030138772 SEQ ID NO: 44 |
| AAV43.23 | 88 | US20030138772 SEQ ID NO: 98 |
| AAV43.25 | 89 | US20030138772 SEQ ID NO: 45 |
| AAV43.25 | 90 | US20030138772 SEQ ID NO: 97 |
| AAV43.5 | 91 | US20030138772 SEQ ID NO: 40 |
| AAV43.5 | 92 | US20030138772 SEQ ID NO: 94 |
| AAV4-4 | 93 | US20150315612 SEQ ID NO: 201 |
| AAV4-4 | 94 | US20150315612 SEQ ID NO: 218 |
| AAV44.1 | 95 | US20030138772 SEQ ID NO: 46 |
| AAV44.1 | 96 | US20030138772 SEQ ID NO: 79 |
| AAV44.5 | 97 | US20030138772 SEQ ID NO: 47 |
| AAV44.5 | 98 | US20030138772 SEQ ID NO: 80 |
| AAV4407 | 99 | US20150315612 SEQ ID NO: 90 |
| AAV5 | 100 | U.S. Pat. No. 7,427,396 SEQ ID NO: 1 |
| AAV5 | 101 | US20030138772 SEQ ID NO: 114 |
| AAV5 | 102 | US20160017295 SEQ ID NO: 5, U.S. Pat. No. 7,427,396 |
| | | SEQ ID NO: 2. US20150315612 SEQ ID NO: 216 |
| AAV5 | 103 | US20150315612 SEQ ID NO: 199 |
| AAV6 | 104 | US20150159173 SEQ ID NO: 13 |
| AAV6 | 105 | US20030138772 SEQ ID NO: 65, US20150159173 SEQ |
| | | ID NO: 29, US20160017295 SEQ ID NO: 6, |
| | | U.S. Pat. No. 6,156,303 SEQ ID NO: 7 |
| AAV6 | 106 | U.S. Pat. No. 6,156,303 SEQ ID NO: 11 |
| AAV6 | 107 | U.S. Pat. No. 6,156,303 SEQ ID NO: 2 |
| AAV6 | 108 | US20150315612 SEQ ID NO: 203 |
| AAV6 | 109 | US20150315612 SEQ ID NO: 220 |
| AAV6.1 | 110 | US20150159173 |
| AAV6.12 | 111 | US20150159173 |
| AAV6.2 | 112 | US20150159173 |
| AAV7 | 113 | US20150159173 SEQ ID NO: 14 |
| AAV7 | 114 | US20150315612 SEQ ID NO: 183 |
| AAV7 | 115 | US20030138772 SEQ ID NO: 2, US20150159173 SEQ |
| | | ID NO: 30, US20150315612 SEQ ID NO: 181, |
| | | US20160017295 SEQ ID NO: 7 |
| AAV7 | 116 | US20030138772 SEQ ID NO: 3 |
| AAV7 | 117 | US20030138772 SEQ ID NO: 1, US20150315612 SEQ |
| | | ID NO: 180 |
| AAV7 | 118 | US20150315612 SEQ ID NO: 213 |
| AAV7 | 119 | US20150315612 SEQ ID NO: 222 |
| AAV8 | 120 | US20150159173 SEQ ID NO: 15 |
| AAV8 | 121 | US20150376240 SEQ ID NO: 7 |
| AAV8 | 122 | US20030138772 SEQ ID NO: 4, US20150315612 SEQ |
| | | ID NO: 182 |
| AAV8 | 123 | US20030138772 SEQ ID NO: 95, US20140359799 SEQ |
| | | ID NO: 1, US20150159173 SEQ ID NO: 31, |
| | | US20160017295 SEQ ID NO: 8, U.S. Pat. No. 7,198,951 |
| | | SEQ ID NO: 7, US20150315612 SEQ ID NO: 223 |
| AAV8 | 124 | US20150376240 SEQ ID NO: 8 |
| AAV8 | 125 | US20150315612 SEQ ID NO: 214 |
| AAV-8b | 126 | US20150376240 SEQ ID NO: 5 |
| AAV-8b | 127 | US20150376240 SEQ ID NO: 3 |
| AAV-8h | 128 | US20150376240 SEQ ID NO: 6 |
| AAV-8h | 129 | US20150376240 SEQ ID NO: 4 |
| AAV9 | 130 | US20030138772 SEQ ID NO: 5 |
| AAV9 | 131 | U.S. Pat. No. 7,198,951 SEQ ID NO: 1 |
| AAV9 | 132 | US20160017295 SEQ ID NO: 9 |
| AAV9 | 133 | US20030138772 SEQ ID NO: 100, U.S. Pat. No. |
| | | 7,198,951 SEQ ID NO: 2 |
| AAV9 | 134 | U.S. Pat. No. 7,198,951 SEQ ID NO: 3 |
| AAV9 (AAVhu.14) | 135 | U.S. Pat. No. 7,906,111 SEQ ID NO: 3; WO2015038958 SEQ ID |
| | | NO: 11 |
| AAV9 (AAVhu.14) | 136 | U.S. Pat. No. 7,906,111 SEQ ID NO: 123; WO2015038958 SEQ ID |
| | | NO: 2 |
| AAVA3.1 | 137 | US20030138772 SEQ ID NO: 120 |
| AAVA3.3 | 138 | US20030138772 SEQ ID NO: 57 |
| AAVA3.3 | 139 | US20030138772 SEQ ID NO: 66 |
| AAVA3.4 | 140 | US20030138772 SEQ ID NO: 54 |
| AAVA3.4 | 141 | US20030138772 SEQ ID NO: 68 |
| AAVA3.5 | 142 | US20030138772 SEQ ID NO: 55 |
| AAVA3.5 | 143 | US20030138772 SEQ ID NO: 69 |
| AAVA3.7 | 144 | US20030138772 SEQ ID NO: 56 |
| AAVA3.7 | 145 | US20030138772 SEQ ID NO: 67 |
| AAV29.3 (AAVbb.1) | 146 | US20030138772 SEQ ID NO: 11 |
| AAVC2 | 147 | US20030138772 SEQ ID NO: 61 |
| AAVCh.5 | 148 | US20150159173 SEQ ID NO: 46, US20150315612 SEQ |
| | | ID NO: 234 |
| AAVcy.2 (AAV13.3) | 149 | US20030138772 SEQ ID NO: 15 |
| AAV24.1 | 150 | US20030138772 SEQ ID NO: 101 |
| AAVcy.3 (AAV24.1) | 151 | US20030138772 SEQ ID NO: 16 |
| AAV27.3 | 152 | US20030138772 SEQ ID NO: 104 |
| AAVcy.4 (AAV27.3) | 153 | US20030138772 SEQ ID NO: 17 |
| AAVcy.5 | 154 | US20150315612 SEQ ID NO: 227 |
| AAV7.2 | 155 | US20030138772 SEQ ID NO: 103 |
| AAVcy.5 (AAV7.2) | 156 | US20030138772 SEQ ID NO: 18 |
| AAV16.3 | 157 | US20030138772 SEQ ID NO: 105 |
| AAVcy.6 (AAV16.3) | 158 | US20030138772 SEQ ID NO: 10 |
| AAVcy.5 | 159 | US20150159173 SEQ ID NO: 8 |
| AAVcy.5 | 160 | US20150159173 SEQ ID NO: 24 |
| AAVCy.5R1 | 161 | US20150159173 |
| AAVCy.5R2 | 162 | US20150159173 |
| AAVCy.5R3 | 163 | US20150159173 |
| AAVCy.5R4 | 164 | US20150159173 |
| AAVDJ | 165 | US20140359799 SEQ ID NO: 3, U.S. Pat. No. 7,588,772 |
| | | SEQ ID NO: 2 |
| AAVDJ | 166 | US20140359799 SEQ ID NO: 2, U.S. Pat. No. 7,588,772 |
| | | SEQ ID NO: 1 |
| AAVDJ-8 | 167 | U.S. Pat. No. 7,588,772; Grimm et al 2008 |
| AAVDJ-8 | 168 | U.S. Pat. No. 7,588,772; Grimm et al 2008 |
| AAVF5 | 169 | US20030138772 SEQ ID NO: 110 |
| AAVH2 | 170 | US20030138772 SEQ ID NO: 26 |
| AAVH6 | 171 | US20030138772 SEQ ID NO: 25 |
| AAVhE1.1 | 172 | U.S. Pat. No. 9,233,131 SEQ ID NO: 44 |
| AAVhEr1.14 | 173 | U.S. Pat. No. 9,233,131 SEQ ID NO: 46 |
| AAVhEr1.16 | 174 | U.S. Pat. No. 9,233,131 SEQ ID NO: 48 |
| AAVhEr1.18 | 175 | U.S. Pat. No. 9,233,131 SEQ ID NO: 49 |
| AAVhEr1.23 (AAVhEr2.29) | 176 | U.S. Pat. No. 9,233,131 SEQ ID NO: 53 |
| AAVhEr1.35 | 177 | U.S. Pat. No. 9,233,131 SEQ ID NO: 50 |
| AAVhEr1.36 | 178 | U.S. Pat. No. 9,233,131 SEQ ID NO: 52 |
| AAVhEr1.5 | 179 | U.S. Pat. No. 9,233,131 SEQ ID NO: 45 |
| AAVhEr1.7 | 180 | U.S. Pat. No. 9,233,131 SEQ ID NO: 51 |
| AAVhEr1.8 | 181 | U.S. Pat. No. 9,233,131 SEQ ID NO: 47 |
| AAVhEr2.16 | 182 | U.S. Pat. No. 9,233,131 SEQ ID NO: 55 |
| AAVhEr2.30 | 183 | U.S. Pat. No. 9,233,131 SEQ ID NO: 56 |
| AAVhEr2.31 | 184 | U.S. Pat. No. 9,233,131 SEQ ID NO: 58 |
| AAVhEr2.36 | 185 | U.S. Pat. No. 9,233,131 SEQ ID NO: 57 |
| AAVhEr2.4 | 186 | U.S. Pat. No. 9,233,131 SEQ ID NO: 54 |
| AAVhEr3.1 | 187 | U.S. Pat. No. 9,233,131 SEQ ID NO: 59 |
| AAVhu.1 | 188 | US20150315612 SEQ ID NO: 46 |
| AAVhu.1 | 189 | US20150315612 SEQ ID NO: 144 |
| AAVhu.10 (AAV16.8) | 190 | US20150315612 SEQ ID NO: 56 |
| AAVhu.10 (AAV16.8) | 191 | US20150315612 SEQ ID NO: 156 |
| AAVhu.11 (AAV16.12) | 192 | US20150315612 SEQ ID NO: 57 |
| AAVhu.11 (AAV16.12) | 193 | US20150315612 SEQ ID NO: 153 |
| AAVhu.12 | 194 | US20150315612 SEQ ID NO: 59 |
| AAVhu.12 | 195 | US20150315612 SEQ ID NO: 154 |
| AAVhu.13 | 196 | US20150159173 SEQ ID NO: 16, US20150315612 SEQ |
| | | ID NO: 71 |
| AAVhu.13 | 197 | US20150159173 SEQ ID NO: 32, US20150315612 SEQ |
| | | ID NO: 129 |
| AAVhu.136.1 | 198 | US20150315612 SEQ ID NO: 165 |
| AAVhu.140.1 | 199 | US20150315612 SEQ ID NO: 166 |
| AAVhu.140.2 | 200 | US20150315612 SEQ ID NO: 167 |
| AAVhu.145.6 | 201 | US20150315612 SEQ ID No: 178 |
| AAVhu.15 | 202 | US20150315612 SEQ ID NO: 147 |
| AAVhu.15 (AAV33.4) | 203 | US20150315612 SEQ ID NO: 50 |
| AAVhu.156.1 | 204 | US20150315612 SEQ ID No: 179 |
| AAVhu.16 | 205 | US20150315612 SEQ ID NO: 148 |
| AAVhu.16 (AAV33.8) | 206 | US20150315612 SEQ ID NO: 51 |
| AAVhu.17 | 207 | US20150315612 SEQ ID NO: 83 |
| AAVhu.17 (AAV33.12) | 208 | US20150315612 SEQ ID NO: 4 |
| AAVhu.172.1 | 209 | US20150315612 SEQ ID NO: 171 |
| AAVhu.172.2 | 210 | US20150315612 SEQ ID NO: 172 |
| AAVhu.173.4 | 211 | US20150315612 SEQ ID NO: 173 |
| AAVhu.173.8 | 212 | US20150315612 SEQ ID NO: 175 |
| AAVhu.18 | 213 | US20150315612 SEQ ID NO: 52 |
| AAVhu.18 | 214 | US20150315612 SEQ ID NO: 149 |
| AAVhu.19 | 215 | US20150315612 SEQ ID NO: 62 |
| AAVhu.19 | 216 | US20150315612 SEQ ID NO: 133 |
| AAVhu.2 | 217 | US20150315612 SEQ ID NO: 48 |
| AAVhu.2 | 218 | US20150315612 SEQ ID NO: 143 |
| AAVhu.20 | 219 | US20150315612 SEQ ID NO: 63 |
| AAVhu.20 | 220 | US20150315612 SEQ ID NO: 134 |
| AAVhu.21 | 221 | US20150315612 SEQ ID NO: 65 |
| AAVhu.21 | 222 | US20150315612 SEQ ID NO: 135 |
| AAVhu.22 | 223 | US20150315612 SEQ ID NO: 67 |
| AAVhu.22 | 224 | US20150315612 SEQ ID NO: 138 |
| AAVhu.23 | 225 | US20150315612 SEQ ID NO: 60 |
| AAVhu.23.2 | 226 | US20150315612 SEQ ID NO: 137 |
| AAVhu.24 | 227 | US20150315612 SEQ ID NO: 66 |
| AAVhu.24 | 228 | US20150315612 SEQ ID NO: 136 |
| AAVhu.25 | 229 | US20150315612 SEQ ID NO: 49 |
| AAVhu.25 | 230 | US20150315612 SEQ ID NO: 146 |
| AAVhu.26 | 231 | US20150159173 SEQ ID NO: 17, US20150315612 SEQ |
| | | ID NO: 61 |
| AAVhu.26 | 232 | US20150159173 SEQ ID NO: 33, US20150315612 SEQ |
| | | ID NO: 139 |
| AAVhu.27 | 233 | US20150315612 SEQ ID NO: 64 |
| AAVhu.27 | 234 | US20150315612 SEQ ID NO: 140 |
| AAVhu.28 | 235 | US20150315612 SEQ ID NO: 68 |
| AAVhu.28 | 236 | US20150315612 SEQ ID NO: 130 |
| AAVhu.29 | 237 | US20150315612 SEQ ID NO: 69 |
| AAVhu.29 | 238 | US20150159173 SEQ ID NO: 42, US20150315612 SEQ |
| | | ID NO: 132 |
| AAVhu.29 | 239 | US20150315612 SEQ ID NO: 225 |
| AAVhu.29R | 240 | US20150159173 |
| AAVhu.3 | 241 | US20150315612 SEQ ID NO: 44 |
| AAVhu.3 | 242 | US20150315612 SEQ ID NO: 145 |
| AAVhu.30 | 243 | US20150315612 SEQ ID NO: 70 |
| AAVhu.30 | 244 | US20150315612 SEQ ID NO: 131 |
| AAVhu.31 | 245 | US20150315612 SEQ ID NO: 1 |
| AAVhu.31 | 246 | US20150315612 SEQ ID NO: 121 |
| AAVhu.32 | 247 | US20150315612 SEQ ID NO: 2 |
| AAVhu.32 | 248 | US20150315612 SEQ ID NO: 122 |
| AAVhu.33 | 249 | US20150315612 SEQ ID NO: 75 |
| AAVhu.33 | 250 | US20150315612 SEQ ID NO: 124 |
| AAVhu.34 | 251 | US20150315612 SEQ ID NO: 72 |
| AAVhu.34 | 252 | US20150315612 SEQ ID NO: 125 |
| AAVhu.35 | 253 | US20150315612 SEQ ID NO: 73 |
| AAVhu.35 | 254 | US20150315612 SEQ ID NO: 164 |
| AAVhu.36 | 255 | US20150315612 SEQ ID NO: 74 |
| AAVhu.36 | 256 | US20150315612 SEQ ID NO: 126 |
| AAVhu.37 | 257 | US20150159173 SEQ ID NO: 34, US20150315612 SEQ |
| | | ID NO: 88 |
| AAVhu.37 (AAV106.1) | 258 | US20150315612 SEQ ID NO: 10, US20150159173 SEQ |
| | | ID NO: 18 |
| AAVhu.38 | 259 | US20150315612 SEQ ID NO: 161 |
| AAVhu.39 | 260 | US20150315612 SEQ ID NO: 102 |
| AAVhu.39 (AAVLG-9) | 261 | US20150315612 SEQ ID NO: 24 |
| AAVhu.4 | 262 | US20150315612 SEQ ID NO: 47 |
| AAVhu.4 | 263 | US20150315612 SEQ ID NO: 141 |
| AAVhu.40 | 264 | US20150315612 SEQ ID NO: 87 |
| AAVhu.40 (AAV114.3) | 265 | US20150315612 SEQ ID No: 11 |
| AAVhu.41 | 266 | US20150315612 SEQ ID NO: 91 |
| AAVhu.41 (AAV127.2) | 267 | US20150315612 SEQ ID NO: 6 |
| AAVhu.42 | 268 | US20150315612 SEQ ID NO: 85 |
| AAVhu.42 (AAV127.5) | 269 | US20150315612 SEQ ID NO: 8 |
| AAVhu.43 | 270 | US20150315612 SEQ ID NO: 160 |
| AAVhu.43 | 271 | US20150315612 SEQ ID NO: 236 |
| AAVhu.43 (AAV128.1) | 272 | US20150315612 SEQ ID NO: 80 |
| AAVhu.44 | 273 | US20150159173 SEQ ID NO: 45, US20150315612 SEQ |
| | | ID NO: 158 |
| AAVhu.44 (AAV128.3) | 274 | US20150315612 SEQ ID NO: 81 |
| AAVhu.44R1 | 275 | US20150159173 |
| AAVhu.44R2 | 276 | US20150159173 |
| AAVhu.44R3 | 277 | US20150159173 |
| AAVhu.45 | 278 | US20150315612 SEQ ID NO: 76 |
| AAVhu.45 | 279 | US20150315612 SEQ ID NO: 127 |
| AAVhu.46 | 280 | US20150315612 SEQ ID NO: 82 |
| AAVhu.46 | 281 | US20150315612 SEQ ID NO: 159 |
| AAVhu.46 | 282 | US20150315612 SEQ ID NO: 224 |
| AAVhu.47 | 283 | US20150315612 SEQ ID NO: 77 |
| AAVhu.47 | 284 | US20150315612 SEQ ID NO: 128 |
| AAVhu.48 | 285 | US20150159173 SEQ ID NO: 38 |
| AAVhu.48 | 286 | US20150315612 SEQ ID NO: 157 |
| AAVhu.48 (AAV130.4) | 287 | US20150315612 SEQ ID NO: 78 |
| AAVhu.48R1 | 288 | US20150159173 |
| AAVhu.48R2 | 289 | US20150159173 |
| AAVhu.48R3 | 290 | US20150159173 |
| AAVhu.49 | 291 | US20150315612 SEQ ID NO: 209 |
| AAVhu.49 | 292 | US20150315612 SEQ ID NO: 189 |
| AAVhu.5 | 293 | US20150315612 SEQ ID NO: 45 |
| AAVhu.5 | 294 | US20150315612 SEQ ID NO: 142 |
| AAVhu.51 | 295 | US20150315612 SEQ ID NO: 208 |
| AAVhu.51 | 296 | US20150315612 SEQ ID NO: 190 |
| AAVhu.52 | 297 | US20150315612 SEQ ID NO: 210 |
| AAVhu.52 | 298 | US20150315612 SEQ ID NO: 191 |
| AAVhu.53 | 299 | US20150159173 SEQ ID NO: 19 |
| AAVhu.53 | 300 | US20150159173 SEQ ID NO: 35 |
| AAVhu.53 (AAV145.1) | 301 | US20150315612 SEQ ID NO: 176 |
| AAVhu.54 | 302 | US20150315612 SEQ ID NO: 188 |
| AAVhu.54 (AAV145.5) | 303 | US20150315612 SEQ ID No: 177 |
| AAVhu.55 | 304 | US20150315612 SEQ ID NO: 187 |
| AAVhu.56 | 305 | US20150315612 SEQ ID NO: 205 |
| AAVhu.56 (AAV145.6) | 306 | US20150315612 SEQ ID NO: 168 |
| AAVhu.56 (AAV145.6) | 307 | US20150315612 SEQ ID NO: 192 |
| AAVhu.57 | 308 | US20150315612 SEQ ID NO: 206 |
| AAVhu.57 | 309 | US20150315612 SEQ ID NO: 169 |
| AAVhu.57 | 310 | US20150315612 SEQ ID NO: 193 |
| AAVhu.58 | 311 | US20150315612 SEQ ID NO: 207 |
| AAVhu.58 | 312 | US20150315612 SEQ ID NO: 194 |
| AAVhu.6 (AAV3.1) | 313 | US20150315612 SEQ ID NO: 5 |
| AAVhu.6 (AAV3.1) | 314 | US20150315612 SEQ ID NO: 84 |
| AAVhu.60 | 315 | US20150315612 SEQ ID NO: 184 |
| AAVhu.60 (AAV161.10) | 316 | US20150315612 SEQ ID NO: 170 |
| AAVhu.61 | 317 | US20150315612 SEQ ID NO: 185 |
| AAVhu.61 (AAV161.6) | 318 | US20150315612 SEQ ID NO: 174 |
| AAVhu.63 | 319 | US20150315612 SEQ ID NO: 204 |
| AAVhu.63 | 320 | US20150315612 SEQ ID NO: 195 |
| AAVhu.64 | 321 | US20150315612 SEQ ID NO: 212 |
| AAVhu.64 | 322 | US20150315612 SEQ ID NO: 196 |
| AAVhu.66 | 323 | US20150315612 SEQ ID NO: 197 |
| AAVhu.67 | 324 | US20150315612 SEQ ID NO: 215 |
| AAVhu.67 | 325 | US20150315612 SEQ ID NO: 198 |
| AAVhu.7 | 326 | US20150315612 SEQ ID NO: 226 |
| AAVhu.7 | 327 | US20150315612 SEQ ID NO: 150 |
| AAVhu.7 (AAV7.3) | 328 | US20150315612 SEQ ID NO: 55 |
| AAVhu.71 | 329 | US20150315612 SEQ ID NO: 79 |
| AAVhu.8 | 330 | US20150315612 SEQ ID NO: 53 |
| AAVhu.8 | 331 | US20150315612 SEQ ID NO: 12 |
| AAVhu.8 | 332 | US20150315612 SEQ ID NO: 151 |
| AAVhu.9 (AAV3.1) | 333 | US20150315612 SEQ ID NO: 58 |
| AAVhu.9 (AAV3.1) | 334 | US20150315612 SEQ ID NO: 155 |
| AAV-LK01 | 335 | US20150376607 SEQ ID NO: 2 |
| AAV-LK01 | 336 | US20150376607 SEQ ID NO: 29 |
| AAV-LK02 | 337 | US20150376607 SEQ ID NO: 3 |
| AAV-LK02 | 338 | US20150376607 SEQ ID NO: 30 |
| AAV-LK03 | 339 | US20150376607 SEQ ID NO: 4 |
| AAV-LK03 | 340 | WO2015121501 SEQ ID NO: 12, US20150376607 SEQ |
| | | ID NO: 31 |
| AAV-LK04 | 341 | US20150376607 SEQ ID NO: 5 |
| AAV-LK04 | 342 | US20150376607 SEQ ID NO: 32 |
| AAV-LK05 | 343 | US20150376607 SEQ ID NO: 6 |
| AAV-LK05 | 344 | US20150376607 SEQ ID NO: 33 |
| AAV-LK06 | 345 | US20150376607 SEQ ID NO: 7 |
| AAV-LK06 | 346 | US20150376607 SEQ ID NO: 34 |
| AAV-LK07 | 347 | US20150376607 SEQ ID NO: 8 |
| AAV-LK07 | 348 | US20150376607 SEQ ID NO: 35 |
| AAV-LK08 | 349 | US20150376607 SEQ ID NO: 9 |
| AAV-LK08 | 350 | US20150376607 SEQ ID NO: 36 |
| AAV-LK09 | 351 | US20150376607 SEQ ID NO: 10 |
| AAV-LK09 | 352 | US20150376607 SEQ ID NO: 37 |
| AAV-LK10 | 353 | US20150376607 SEQ ID NO: 11 |
| AAV-LK10 | 354 | US20150376607 SEQ ID NO: 38 |
| AAV-LK11 | 355 | US20150376607 SEQ ID NO: 12 |
| AAV-LK11 | 356 | US20150376607 SEQ ID NO: 39 |
| AAV-LK12 | 357 | US20150376607 SEQ ID NO: 13 |
| AAV-LK12 | 358 | US20150376607 SEQ ID NO: 40 |
| AAV-LK13 | 359 | US20150376607 SEQ ID NO: 14 |
| AAV-LK13 | 360 | US20150376607 SEQ ID NO: 41 |
| AAV-LK14 | 361 | US20150376607 SEQ ID NO: 15 |
| AAV-LK14 | 362 | US20150376607 SEQ ID NO: 42 |
| AAV-LK15 | 363 | US20150376607 SEQ ID NO: 16 |
| AAV-LK15 | 364 | US20150376607 SEQ ID NO: 43 |
| AAV-LK16 | 365 | US20150376607 SEQ ID NO: 17 |
| AAV-LK16 | 366 | US20150376607 SEQ ID NO: 44 |
| AAV-LK17 | 367 | US20150376607 SEQ ID NO: 18 |
| AAV-LK17 | 368 | US20150376607 SEQ ID NO: 45 |
| AAV-LK18 | 369 | US20150376607 SEQ ID NO: 19 |
| AAV-LK18 | 370 | US20150376607 SEQ ID NO: 46 |
| AAV-LK19 | 371 | US20150376607 SEQ ID NO: 20 |
| AAV-LK19 | 372 | US20150376607 SEQ ID NO: 47 |
| AAV-PAEC | 373 | US20150376607 SEQ ID NO: 1 |
| AAV-PAEC | 374 | US20150376607 SEQ ID NO: 48 |
| AAV-PAEC11 | 375 | US20150376607 SEQ ID NO: 26 |
| AAV-PAEC11 | 376 | US20150376607 SEQ ID NO: 54 |
| AAV-PAEC12 | 377 | US20150376607 SEQ ID NO: 27 |
| AAV-PAEC12 | 378 | US20150376607 SEQ ID NO: 51 |
| AAV-PAEC13 | 379 | US20150376607 SEQ ID NO: 28 |
| AAV-PAEC13 | 380 | US20150376607 SEQ ID NO: 49 |
| AAV-PAEC2 | 381 | US20150376607 SEQ ID NO: 21 |
| AAV-PAEC2 | 382 | US20150376607 SEQ ID NO: 56 |
| AAV-PAEC4 | 383 | US20150376607 SEQ ID NO: 22 |
| AAV-PAEC4 | 384 | US20150376607 SEQ ID NO: 55 |
| AAV-PAEC6 | 385 | US20150376607 SEQ ID NO: 23 |
| AAV-PAEC6 | 386 | US20150376607 SEQ ID NO: 52 |
| AAV-PAEC7 | 387 | US20150376607 SEQ ID NO: 24 |
| AAV-PAEC7 | 388 | US20150376607 SEQ ID NO: 53 |
| AAV-PAEC8 | 389 | US20150376607 SEQ ID NO: 25 |
| AAV-PAEC8 | 390 | US20150376607 SEQ ID NO: 50 |
| AAVpi.1 | 391 | US20150315612 SEQ ID NO: 28 |
| AAVpi.1 | 392 | US20150315612 SEQ ID NO: 93 |
| AAVpi.2 | 393 | US20150315612 SEQ ID NO: 30 |
| AAVpi.2 | 394 | US20150315612 SEQ ID NO: 95 |
| AAVpi.3 | 395 | US20150315612 SEQ ID NO: 29 |
| AAVpi.3 | 396 | US20150315612 SEQ ID NO: 94 |
| AAVrh.10 | 397 | US20150159173 SEQ ID NO: 9 |
| AAVrh.10 | 398 | US20150159173 SEQ ID NO: 25 |
| AAV44.2 | 399 | US20030138772 SEQ ID NO: 59 |
| AAVrh.10 (AAV44.2) | 400 | US20030138772 SEQ ID NO: 81 |
| AAV42.1B | 401 | US20030138772 SEQ ID NO: 90 |
| AAVrh.12 (AAV42.1b) | 402 | US20030138772 SEQ ID NO: 30 |
| AAVrh.13 | 403 | US20150159173 SEQ ID NO: 10 |
| AAVrh.13 | 404 | US20150159173 SEQ ID NO: 26 |
| AAVrh.13 | 405 | US20150315612 SEQ ID NO: 228 |
| AAVrh.13R | 406 | US20150159173 |
| AAV42.3A | 407 | US20030138772 SEQ ID NO: 87 |
| AAVrh.14 (AAV42.3a) | 408 | US20030138772 SEQ ID NO: 32 |
| AAV42.5A | 409 | US20030138772 SEQ ID NO: 89 |
| AAVrh.17 (AAV42.5a) | 410 | US20030138772 SEQ ID NO: 34 |
| AAV42.5B | 411 | US20030138772 SEQ ID NO: 91 |
| AAVrh.18 (AAV42.5b) | 412 | US20030138772 SEQ ID NO: 29 |
| AAV42.6B | 413 | US20030138772 SEQ ID NO: 112 |
| AAVrh.19 (AAV42.6b) | 414 | US20030138772 SEQ ID NO: 38 |
| AAVrh.2 | 415 | US20150159173 SEQ ID NO: 39 |
| AAVrh.2 | 416 | US20150315612 SEQ ID NO: 231 |
| AAVrh.20 | 417 | US20150159173 SEQ ID NO: 1 |
| AAV42.10 | 418 | US20030138772 SEQ ID NO: 106 |
| AAVrh.21 (AAV42.10) | 419 | US20030138772 SEQ ID NO: 35 |
| AAV42.11 | 420 | US20030138772 SEQ ID NO: 108 |
| AAVrh.22 (AAV42.11) | 421 | US20030138772 SEQ ID NO: 37 |
| AAV42.12 | 422 | US20030138772 SEQ ID NO: 113 |
| AAVrh.23 (AAV42.12) | 423 | US20030138772 SEQ ID NO: 58 |
| AAV42.13 | 424 | US20030138772 SEQ ID NO: 86 |
| AAVrh.24 (AAV42.13) | 425 | US20030138772 SEQ ID NO: 31 |
| AAV42.15 | 426 | US20030138772 SEQ ID NO: 84 |
| AAVrh.25 (AAV42.15) | 427 | US20030138772 SEQ ID NO: 28 |
| AAVrh.2R | 428 | US20150159173 |
| AAVrh.31 (AAV223.1) | 429 | US20030138772 SEQ ID NO: 48 |
| AAVC1 | 430 | US20030138772 SEQ ID NO: 60 |
| AAVrh.32 (AAVC1) | 431 | US20030138772 SEQ ID NO: 19 |
| AAVrh.32/33 | 432 | US20150159173 SEQ ID NO: 2 |
| AAVrh.33 (AAVC3) | 433 | US20030138772 SEQ ID NO: 20 |
| AAVC5 | 434 | US20030138772 SEQ ID NO: 62 |
| AAVrh.34 (AAVC5) | 435 | US20030138772 SEQ ID NO: 21 |
| AAVF1 | 436 | US20030138772 SEQ ID NO: 109 |
| AAVrh.35 (AAVF1) | 437 | US20030138772 SEQ ID NO: 22 |
| AAVF3 | 438 | US20030138772 SEQ ID NO: 111 |
| AAVrh.36 (AAVF3) | 439 | US20030138772 SEQ ID NO: 23 |
| AAVrh.37 | 440 | US20030138772 SEQ ID NO: 24 |
| AAVrh.37 | 441 | US20150159173 SEQ ID NO: 40 |
| AAVrh.37 | 442 | US20150315612 SEQ ID NO: 229 |
| AAVrh.37R2 | 443 | US20150159173 |
| AAVrh.38 (AAVLG-4) | 444 | US20150315612 SEQ ID NO: 7 |
| AAVrh.38 (AAVLG-4) | 445 | US20150315612 SEQ ID NO: 86 |
| AAVrh.39 | 446 | US20150159173 SEQ ID NO: 20, US20150315612 SEQ |
| | | ID NO: 13 |
| AAVrh.39 | 447 | US20150159173 SEQ ID NO: 3, US20150159173 SEQ |
| | | ID NO: 36, US20150315612 SEQ ID NO: 89 |
| AAVrh.40 | 448 | US20150315612 SEQ ID NO: 92 |
| AAVrh.40 (AAVLG-10) | 449 | US20150315612 SEQ ID No: 14 |
| AAVrh.43 (AAVN721-8) | 450 | US20150315612 SEQ ID NO: 43, US20150159173 SEQ |
| | | ID NO: 21 |
| AAVrh.43 (AAVN721-8) | 451 | US20150315612 SEQ ID NO: 163, US20150159173 |
| | | SEQ ID NO: 37 |
| AAVrh.44 | 452 | US20150315612 SEQ ID NO: 34 |
| AAVrh.44 | 453 | US20150315612 SEQ ID NO: 111 |
| AAVrh.45 | 454 | US20150315612 SEQ ID NO: 41 |
| AAVrh.45 | 455 | US20150315612 SEQ ID NO: 109 |
| AAVrh.46 | 456 | US20150159173 SEQ ID NO: 22, US20150315612 SEQ |
| | | ID NO: 19 |
| AAVrh.46 | 457 | US20150159173 SEQ ID NO: 4, US20150315612 SEQ |
| | | ID NO: 101 |
| AAVrh.47 | 458 | US20150315612 SEQ ID NO: 38 |
| AAVrh.47 | 459 | US20150315612 SEQ ID NO: 118 |
| AAVrh.48 | 460 | US20150159173 SEQ ID NO: 44, US20150315612 SEQ |
| | | ID NO: 115 |
| AAVrh.48.1 | 461 | US20150159173 |
| AAVrh.48.1.2 | 462 | US20150159173 |
| AAVrh.48.2 | 463 | US20150159173 |
| AAVrh.48 (AAV1-7) | 464 | US20150315612 SEQ ID NO: 32 |
| AAVrh.49 (AAV1-8) | 465 | US20150315612 SEQ ID NO: 25 |
| AAVrh.49 (AAV1-8) | 466 | US20150315612 SEQ ID NO: 103 |
| AAVrh.50 (AAV2-4) | 467 | US20150315612 SEQ ID NO: 23 |
| AAVrh.50 (AAV2-4) | 468 | US20150315612 SEQ ID NO: 108 |
| AAVrh.51 (AAV2-5) | 469 | US20150315612 SEQ ID No: 22 |
| AAVrh.51 (AAV2-5) | 470 | US20150315612 SEQ ID NO: 104 |
| AAVrh.52 (AAV3-9) | 471 | US20150315612 SEQ ID NO: 18 |
| AAVrh.52 (AAV3-9) | 472 | US20150315612 SEQ ID NO: 96 |
| AAVrh.53 | 473 | US20150315612 SEQ ID NO: 97 |
| AAVrh.53 (AAV3-11) | 474 | US20150315612 SEQ ID NO: 17 |
| AAVrh.53 (AAV3-11) | 475 | US20150315612 SEQ ID NO: 186 |
| AAVrh.54 | 476 | US20150315612 SEQ ID NO: 40 |
| AAVrh.54 | 477 | US20150159173 SEQ ID NO: 49, US20150315612 SEQ |
| | | ID NO: 116 |
| AAVrh.55 | 478 | US20150315612 SEQ ID NO: 37 |
| AAVrh.55 (AAV4-19) | 479 | US20150315612 SEQ ID NO: 117 |
| AAVrh.56 | 480 | US20150315612 SEQ ID NO: 54 |
| AAVrh.56 | 481 | US20150315612 SEQ ID NO: 152 |
| AAVrh.57 | 482 | US20150315612 SEQ ID NO: 26 |
| AAVrh.57 | 483 | US20150315612 SEQ ID NO: 105 |
| AAVrh.58 | 484 | US20150315612 SEQ ID NO: 27 |
| AAVrh.58 | 485 | US20150159173 SEQ ID NO: 48, US20150315612 SEQ |
| | | ID NO: 106 |
| AAVrh.58 | 486 | US20150315612 SEQ ID NO: 232 |
| AAVrh.59 | 487 | US20150315612 SEQ ID NO: 42 |
| AAVrh.59 | 488 | US20150315612 SEQ ID NO: 110 |
| AAVrh.60 | 489 | US20150315612 SEQ ID NO: 31 |
| AAVrh.60 | 490 | US20150315612 SEQ ID NO: 120 |
| AAVrh.61 | 491 | US20150315612 SEQ ID NO: 107 |
| AAVrh.61 (AAV2-3) | 492 | US20150315612 SEQ ID NO: 21 |
| AAVrh.62 (AAV2-15) | 493 | US20150315612 SEQ ID No: 33 |
| AAVrh.62 (AAV2-15) | 494 | US20150315612 SEQ ID NO: 114 |
| AAVrh.64 | 495 | US20150315612 SEQ ID No: 15 |
| AAVrh.64 | 496 | US20150159173 SEQ ID NO: 43, US20150315612 SEQ |
| | | ID NO: 99 |
| AAVrh.64 | 497 | US20150315612 SEQ ID NO: 233 |
| AAVRh.64R1 | 498 | US20150159173 |
| AAVRh.64R2 | 499 | US20150159173 |
| AAVrh.65 | 500 | US20150315612 SEQ ID NO: 35 |
| AAVrh.65 | 501 | US20150315612 SEQ ID NO: 112 |
| AAVrh.67 | 502 | US20150315612 SEQ ID NO: 36 |
| AAVrh.67 | 503 | US20150315612 SEQ ID NO: 230 |
| AAVrh.67 | 504 | US20150159173 SEQ ID NO: 47, US20150315612 SEQ |
| | | ID NO: 113 |
| AAVrh.68 | 505 | US20150315612 SEQ ID NO: 16 |
| AAVrh.68 | 506 | US20150315612 SEQ ID NO: 100 |
| AAVrh.69 | 507 | US20150315612 SEQ ID NO: 39 |
| AAVrh.69 | 508 | US20150315612 SEQ ID NO: 119 |
| AAVrh.70 | 509 | US20150315612 SEQ ID NO: 20 |
| AAVrh.70 | 510 | US20150315612 SEQ ID NO: 98 |
| AAVrh.71 | 511 | US20150315612 SEQ ID NO: 162 |
| AAVrh.72 | 512 | US20150315612 SEQ ID NO: 9 |
| AAVrh.73 | 513 | US20150159173 SEQ ID NO: 5 |
| AAVrh.74 | 514 | US20150159173 SEQ ID NO: 6 |
| AAVrh.8 | 515 | US20150159173 SEQ ID NO: 41 |
| AAVrh.8 | 516 | US20150315612 SEQ ID NO: 235 |
| AAVrh.8R | 517 | US20150159173, WO2015168666 SEQ ID NO: 9 |
| AAVrh.8R A586R mutant | 518 | WO2015168666 SEQ ID NO: 10 |
| AAVrh.8R R533A mutant | 519 | WO2015168666 SEQ ID NO: 11 |
| BAAV (bovine AAV) | 520 | U.S. Pat. No. 9,193,769 SEQ ID NO: 8 |
| BAAV (bovine AAV) | 521 | U.S. Pat. No. 9,193,769 SEQ ID NO: 10 |
| BAAV (bovine AAV) | 522 | U.S. Pat. No. 9,193,769 SEQ ID NO: 4 |
| BAAV (bovine AAV) | 523 | U.S. Pat. No. 9,193,769 SEQ ID NO: 2 |
| BAAV (bovine AAV) | 524 | U.S. Pat. No. 9,193,769 SEQ ID NO: 6 |
| BAAV (bovine AAV) | 525 | U.S. Pat. No. 9,193,769 SEQ ID NO: 1 |
| BAAV (bovine AAV) | 526 | U.S. Pat. No. 9,193,769 SEQ ID NO: 5 |
| BAAV (bovine AAV) | 527 | U.S. Pat. No. 9,193,769 SEQ ID NO: 3 |
| BAAV (bovine AAV) | 528 | U.S. Pat. No. 9,193,769 SEQ ID NO: 11 |
| BAAV (bovine AAV) | 529 | U.S. Pat. No. 7,427,396 SEQ ID NO: 5 |
| BAAV (bovine AAV) | 530 | U.S. Pat. No. 7,427,396 SEQ ID NO: 6 |
| BAAV (bovine AAV) | 531 | U.S. Pat. No. 9,193,769 SEQ ID NO: 7 |
| BAAV (bovine AAV) | 532 | U.S. Pat. No. 9,193,769 SEQ ID NO: 9 |
| BNP61 AAV | 533 | US20150238550 SEQ ID NO: 1 |
| BNP61 AAV | 534 | US20150238550 SEQ ID NO: 2 |
| BNP62 AAV | 535 | US20150238550 SEQ ID NO: 3 |
| BNP63 AAV | 536 | US20150238550 SEQ ID NO: 4 |
| caprine AAV | 537 | U.S. Pat. No. 7,427,396 SEQ ID NO: 3 |
| caprine AAV | 538 | U.S. Pat. No. 7,427,396 SEQ ID NO: 4 |
| true type AAV (ttAAV) | 539 | WO2015121501 SEQ ID NO: 2 |
| AAAV (Avian AAV) | 540 | U.S. Pat. No. 9,238,800 SEQ ID NO: 12 |
| AAAV (Avian AAV) | 541 | U.S. Pat. No. 9,238,800 SEQ ID NO: 2 |
| AAAV (Avian AAV) | 542 | U.S. Pat. No. 9,238,800 SEQ ID NO: 6 |
| AAAV (Avian AAV) | 543 | U.S. Pat. No. 9,238,800 SEQ ID NO: 4 |
| AAAV (Avian AAV) | 544 | U.S. Pat. No. 9,238,800 SEQ ID NO: 8 |
| AAAV (Avian AAV) | 545 | U.S. Pat. No. 9,238,800 SEQ ID NO: 14 |
| AAAV (Avian AAV) | 546 | U.S. Pat. No. 9,238,800 SEQ ID NO: 10 |
| AAAV (Avian AAV) | 547 | U.S. Pat. No. 9,238,800 SEQ ID NO: 15 |
| AAAV (Avian AAV) | 548 | U.S. Pat. No. 9,238,800 SEQ ID NO: 5 |
| AAAV (Avian AAV) | 549 | U.S. Pat. No. 9,238,800 SEQ ID NO: 9 |
| AAAV (Avian AAV) | 550 | U.S. Pat. No. 9,238,800 SEQ ID NO: 3 |
| AAAV (Avian AAV) | 551 | U.S. Pat. No. 9,238,800 SEQ ID NO: 7 |
| AAAV (Avian AAV) | 552 | U.S. Pat. No. 9,238,800 SEQ ID NO: 11 |
| AAAV (Avian AAV) | 553 | U.S. Pat. No. 9,238,800 SEQ ID NO: 13 |
| AAAV (Avian AAV) | 554 | U.S. Pat. No. 9,238,800 SEQ ID NO: 1 |
| AAV Shuffle 100-1 | 555 | US20160017295 SEQ ID NO: 23 |
| AAV Shuffle 100-1 | 556 | US20160017295 SEQ ID NO: 11 |
| AAV Shuffle 100-2 | 557 | US20160017295 SEQ ID NO: 37 |
| AAV Shuffle 100-2 | 558 | US20160017295 SEQ ID NO: 29 |
| AAV Shuffle 100-3 | 559 | US20160017295 SEQ ID NO: 24 |
| AAV Shuffle 100-3 | 560 | US20160017295 SEQ ID NO: 12 |
| AAV Shuffle 100-7 | 561 | US20160017295 SEQ ID NO: 25 |
| AAV Shuffle 100-7 | 562 | US20160017295 SEQ ID NO: 13 |
| AAV Shuffle 10-2 | 563 | US20160017295 SEQ ID NO: 34 |
| AAV Shuffle 10-2 | 564 | US20160017295 SEQ ID NO: 26 |
| AAV Shuffle 10-6 | 565 | US20160017295 SEQ ID NO: 35 |
| AAV Shuffle 10-6 | 566 | US20160017295 SEQ ID NO: 27 |
| AAV Shuffle 10-8 | 567 | US20160017295 SEQ ID NO: 36 |
| AAV Shuffle 10-8 | 568 | US20160017295 SEQ ID NO: 28 |
| AAV SM 100-10 | 569 | US20160017295 SEQ ID NO: 41 |
| AAV SM 100-10 | 570 | US20160017295 SEQ ID NO: 33 |
| AAV SM 100-3 | 571 | US20160017295 SEQ ID NO: 40 |
| AAV SM 100-3 | 572 | US20160017295 SEQ ID NO: 32 |
| AAV SM 10-1 | 573 | US20160017295 SEQ ID NO: 38 |
| AAV SM 10-1 | 574 | US20160017295 SEQ ID NO: 30 |
| AAV SM 10-2 | 575 | US20160017295 SEQ ID NO: 10 |
| AAV SM 10-2 | 576 | US20160017295 SEQ ID NO: 22 |
| AAV SM 10-8 | 577 | US20160017295 SEQ ID NO: 39 |
| AAV SM 10-8 | 578 | US20160017295 SEQ ID NO: 31 |
| AAVF1/HSC1 | 579 | WO2016049230 SEQ ID NO: 20 |
| AAVF2/HSC2 | 580 | WO2016049230 SEQ ID NO: 21 |
| AAVF3/HSC3 | 581 | WO2016049230 SEQ ID NO: 22 |
| AAVF4/HSC4 | 582 | WO2016049230 SEQ ID NO: 23 |
| AAVF5/HSC5 | 583 | WO2016049230 SEQ ID NO: 25 |
| AAVF6/HSC6 | 584 | WO2016049230 SEQ ID NO: 24 |
| AAVF7/HSC7 | 585 | WO2016049230 SEQ ID NO: 27 |
| AAVF8/HSC8 | 586 | WO2016049230 SEQ ID NO: 28 |
| AAVF9/HSC9 | 587 | WO2016049230 SEQ ID NO: 29 |
| AAVF11/HSC11 | 588 | WO2016049230 SEQ ID NO: 26 |
| AAVF12/HSC12 | 589 | WO2016049230 SEQ ID NO: 30 |
| AAVF13/HSC13 | 590 | WO2016049230 SEQ ID NO: 31 |
| AAVF14/HSC14 | 591 | WO2016049230 SEQ ID NO: 32 |
| AAVF15/HSC15 | 592 | WO2016049230 SEQ ID NO: 33 |
| AAVF16/HSC16 | 593 | WO2016049230 SEQ ID NO: 34 |
| AAVF17/HSC17 | 594 | WO2016049230 SEQ ID NO: 35 |
| AAVF1/HSC1 | 595 | WO2016049230 SEQ ID NO: 2 |
| AAVF2/HSC2 | 596 | WO2016049230 SEQ ID NO: 3 |
| AAVF3/HSC3 | 597 | WO2016049230 SEQ ID NO: 5 |
| AAVF4/HSC4 | 598 | WO2016049230 SEQ ID NO: 6 |
| AAVF5/HSC5 | 599 | WO2016049230 SEQ ID NO: 11 |
| AAVF6/HSC6 | 600 | WO2016049230 SEQ ID NO: 7 |
| AAVF7/HSC7 | 601 | WO2016049230 SEQ ID NO: 8 |
| AAVF8/HSC8 | 602 | WO2016049230 SEQ ID NO: 9 |
| AAVF9/HSC9 | 603 | WO2016049230 SEQ ID NO: 10 |
| AAVF11/HSC11 | 604 | WO2016049230 SEQ ID NO: 4 |
| AAVF12/HSC12 | 605 | WO2016049230 SEQ ID NO: 12 |
| AAVF13/HSC13 | 606 | WO2016049230 SEQ ID NO: 14 |
| AAVF14/HSC14 | 607 | WO2016049230 SEQ ID NO: 15 |
| AAVF15/HSC15 | 608 | WO2016049230 SEQ ID NO: 16 |
| AAVF16/HSC16 | 609 | WO2016049230 SEQ ID NO: 17 |
| AAVF17/HSC17 | 610 | WO2016049230 SEQ ID NO: 13 |
| AAV CBr-E1 | 611 | U.S. Pat. No. 8,734,809 SEQ ID NO: 13 |
| AAV CBr-E2 | 612 | U.S. Pat. No. 8,734,809 SEQ ID NO: 14 |
| AAV CBr-E3 | 613 | U.S. Pat. No. 8,734,809 SEQ ID NO: 15 |
| AAV CBr-E4 | 614 | U.S. Pat. No. 8,734,809 SEQ ID NO: 16 |
| AAV CBr-E5 | 615 | U.S. Pat. No. 8,734,809 SEQ ID NO: 17 |
| AAV CBr-e5 | 616 | U.S. Pat. No. 8,734,809 SEQ ID NO: 18 |
| AAV CBr-E6 | 617 | U.S. Pat. No. 8,734,809 SEQ ID NO: 19 |
| AAV CBr-E7 | 618 | U.S. Pat. No. 8,734,809 SEQ ID NO: 20 |
| AAV CBr-E8 | 619 | U.S. Pat. No. 8,734,809 SEQ ID NO: 21 |
| AAV CLv-D1 | 620 | U.S. Pat. No. 8,734,809 SEQ ID NO: 22 |
| AAV CLv-D2 | 621 | U.S. Pat. No. 8,734,809 SEQ ID NO: 23 |
| AAV CLv-D3 | 622 | U.S. Pat. No. 8,734,809 SEQ ID NO: 24 |
| AAV CLv-D4 | 623 | U.S. Pat. No. 8,734,809 SEQ ID NO: 25 |
| AAV CLv-D5 | 624 | U.S. Pat. No. 8,734,809 SEQ ID NO: 26 |
| AAV CLv-D6 | 625 | U.S. Pat. No. 8,734,809 SEQ ID NO: 27 |
| AAV CLv-D7 | 626 | U.S. Pat. No. 8,734,809 SEQ ID NO: 28 |
| AAV CLv-D8 | 627 | U.S. Pat. No. 8,734,809 SEQ ID NO: 29 |
| AAV CLv-E1 | 628 | U.S. Pat. No. 8,734,809 SEQ ID NO: 13 |
| AAV CLv-R1 | 629 | U.S. Pat. No. 8,734,809 SEQ ID NO: 30 |
| AAV CLv-R2 | 630 | U.S. Pat. No. 8,734,809 SEQ ID NO: 31 |
| AAV CLv-R3 | 631 | U.S. Pat. No. 8,734,809 SEQ ID NO: 32 |
| AAV CLv-R4 | 632 | U.S. Pat. No. 8,734,809 SEQ ID NO: 33 |
| AAV CLv-R5 | 633 | U.S. Pat. No. 8,734,809 SEQ ID NO: 34 |
| AAV CLv-R6 | 634 | U.S. Pat. No. 8,734,809 SEQ ID NO: 35 |
| AAV CLv-R7 | 635 | U.S. Pat. No. 8,734,809 SEQ ID NO: 36 |
| AAV CLv-R8 | 636 | U.S. Pat. No. 8,734,809 SEQ ID NO: 37 |
| AAV CLv-R9 | 637 | U.S. Pat. No. 8,734,809 SEQ ID NO: 38 |
| AAV CLg-F1 | 638 | U.S. Pat. No. 8,734,809 SEQ ID NO: 39 |
| AAV CLg-F2 | 639 | U.S. Pat. No. 8,734,809 SEQ ID NO: 40 |
| AAV CLg-F3 | 640 | U.S. Pat. No. 8,734,809 SEQ ID NO: 41 |
| AAV CLg-F4 | 641 | U.S. Pat. No. 8,734,809 SEQ ID NO: 42 |
| AAV CLg-F5 | 642 | U.S. Pat. No. 8,734,809 SEQ ID NO: 43 |
| AAV CLg-F6 | 643 | U.S. Pat. No. 8,734,809 SEQ ID NO: 43 |
| AAV CLg-F7 | 644 | U.S. Pat. No. 8,734,809 SEQ ID NO: 44 |
| AAV CLg-F8 | 645 | U.S. Pat. No. 8,734,809 SEQ ID NO: 43 |
| AAV CSp-1 | 646 | U.S. Pat. No. 8,734,809 SEQ ID NO: 45 |
| AAV CSp-10 | 647 | U.S. Pat. No. 8,734,809 SEQ ID NO: 46 |
| AAV CSp-11 | 648 | U.S. Pat. No. 8,734,809 SEQ ID NO: 47 |
| AAV CSp-2 | 649 | U.S. Pat. No. 8,734,809 SEQ ID NO: 48 |
| AAV CSp-3 | 650 | U.S. Pat. No. 8,734,809 SEQ ID NO: 49 |
| AAV CSp-4 | 651 | U.S. Pat. No. 8,734,809 SEQ ID NO: 50 |
| AAV CSp-6 | 652 | U.S. Pat. No. 8,734,809 SEQ ID NO: 51 |
| AAV CSp-7 | 653 | U.S. Pat. No. 8,734,809 SEQ ID NO: 52 |
| AAV CSp-8 | 654 | U.S. Pat. No. 8,734,809 SEQ ID NO: 53 |
| AAV CSp-9 | 655 | U.S. Pat. No. 8,734,809 SEQ ID NO: 54 |
| AAV CHt-2 | 656 | U.S. Pat. No. 8,734,809 SEQ ID NO: 55 |
| AAV CHt-3 | 657 | U.S. Pat. No. 8,734,809 SEQ ID NO: 56 |
| AAV CKd-1 | 658 | U.S. Pat. No. 8,734,809 SEQ ID NO: 57 |
| AAV CKd-10 | 659 | U.S. Pat. No. 8,734,809 SEQ ID NO: 58 |
| AAV CKd-2 | 660 | U.S. Pat. No. 8,734,809 SEQ ID NO: 59 |
| AAV CKd-3 | 661 | U.S. Pat. No. 8,734,809 SEQ ID NO: 60 |
| AAV CKd-4 | 662 | U.S. Pat. No. 8,734,809 SEQ ID NO: 61 |
| AAV CKd-6 | 663 | U.S. Pat. No. 8,734,809 SEQ ID NO: 62 |
| AAV CKd-7 | 664 | U.S. Pat. No. 8,734,809 SEQ ID NO: 63 |
| AAV CKd-8 | 665 | U.S. Pat. No. 8,734,809 SEQ ID NO: 64 |
| AAV CLv-1 | 666 | U.S. Pat. No. 8,734,809 SEQ ID NO: 65 |
| AAV CLv-12 | 667 | U.S. Pat. No. 8,734,809 SEQ ID NO: 66 |
| AAV CLv-13 | 668 | U.S. Pat. No. 8,734,809 SEQ ID NO: 67 |
| AAV CLv-2 | 669 | U.S. Pat. No. 8,734,809 SEQ ID NO: 68 |
| AAV CLv-3 | 670 | U.S. Pat. No. 8,734,809 SEQ ID NO: 69 |
| AAV CLv-4 | 671 | U.S. Pat. No. 8,734,809 SEQ ID NO: 70 |
| AAV CLv-6 | 672 | U.S. Pat. No. 8,734,809 SEQ ID NO: 71 |
| AAV CLv-8 | 673 | U.S. Pat. No. 8,734,809 SEQ ID NO: 72 |
| AAV CKd-B1 | 674 | U.S. Pat. No. 8,734,809 SEQ ID NO: 73 |
| AAV CKd-B2 | 675 | U.S. Pat. No. 8,734,809 SEQ ID NO: 74 |
| AAV CKd-B3 | 676 | U.S. Pat. No. 8,734,809 SEQ ID NO: 75 |
| AAV CKd-B4 | 677 | U.S. Pat. No. 8,734,809 SEQ ID NO: 76 |
| AAV CKd-B5 | 678 | U.S. Pat. No. 8,734,809 SEQ ID NO: 77 |
| AAV CKd-B6 | 679 | U.S. Pat. No. 8,734,809 SEQ ID NO: 78 |
| AAV CKd-B7 | 680 | U.S. Pat. No. 8,734,809 SEQ ID NO: 79 |
| AAV CKd-B8 | 681 | U.S. Pat. No. 8,734,809 SEQ ID NO: 80 |
| AAV CKd-H1 | 682 | U.S. Pat. No. 8,734,809 SEQ ID NO: 81 |
| AAV CKd-H2 | 683 | U.S. Pat. No. 8,734,809 SEQ ID NO: 82 |
| AAV CKd-H3 | 684 | U.S. Pat. No. 8,734,809 SEQ ID NO: 83 |
| AAV CKd-H4 | 685 | U.S. Pat. No. 8,734,809 SEQ ID NO: 84 |
| AAV CKd-H5 | 686 | U.S. Pat. No. 8,734,809 SEQ ID NO: 85 |
| AAV CKd-H6 | 687 | U.S. Pat. No. 8,734,809 SEQ ID NO: 77 |
| AAV CHt-1 | 688 | U.S. Pat. No. 8,734,809 SEQ ID NO: 86 |
| AAV CLv1-1 | 689 | U.S. Pat. No. 8,734,809 SEQ ID NO: 171 |
| AAV CLv1-2 | 690 | U.S. Pat. No. 8,734,809 SEQ ID NO: 172 |
| AAV CLv1-3 | 691 | U.S. Pat. No. 8,734,809 SEQ ID NO: 173 |
| AAV CLv1-4 | 692 | U.S. Pat. No. 8,734,809 SEQ ID NO: 174 |
| AAV Clv1-7 | 693 | U.S. Pat. No. 8,734,809 SEQ ID NO: 175 |
| AAV Clv1-8 | 694 | U.S. Pat. No. 8,734,809 SEQ ID NO: 176 |
| AAV Clv1-9 | 695 | U.S. Pat. No. 8,734,809 SEQ ID NO: 177 |
| AAV Clv1-10 | 696 | U.S. Pat. No. 8,734,809 SEQ ID NO: 178 |
| AAV.VR-355 | 697 | U.S. Pat. No. 8,734,809 SEQ ID NO: 181 |
| AAV.hu.48R3 | 698 | U.S. Pat. No. 8,734,809 SEQ ID NO: 183 |
| AAV CBr-E1 | 699 | U.S. Pat. No. 8,734,809 SEQ ID NO: 87 |
| AAV CBr-E2 | 700 | U.S. Pat. No. 8,734,809 SEQ ID NO: 88 |
| AAV CBr-E3 | 701 | U.S. Pat. No. 8,734,809 SEQ ID NO: 89 |
| AAV CBr-E4 | 702 | U.S. Pat. No. 8,734,809 SEQ ID NO: 90 |
| AAV CBr-E5 | 703 | U.S. Pat. No. 8,734,809 SEQ ID NO: 91 |
| AAV CBr-e5 | 704 | U.S. Pat. No. 8,734,809 SEQ ID NO: 92 |
| AAV CBr-E6 | 705 | U.S. Pat. No. 8,734,809 SEQ ID NO: 93 |
| AAV CBr-E7 | 706 | U.S. Pat. No. 8,734,809 SEQ ID NO: 94 |
| AAV CBr-E8 | 707 | U.S. Pat. No. 8,734,809 SEQ ID NO: 95 |
| AAV CLv-D1 | 708 | U.S. Pat. No. 8,734,809 SEQ ID NO: 96 |
| AAV CLv-D2 | 709 | U.S. Pat. No. 8,734,809 SEQ ID NO: 97 |
| AAV CLv-D3 | 710 | U.S. Pat. No. 8,734,809 SEQ ID NO: 98 |
| AAV CLv-D4 | 711 | U.S. Pat. No. 8,734,809 SEQ ID NO: 99 |
| AAV CLv-D5 | 712 | U.S. Pat. No. 8,734,809 SEQ ID NO: 100 |
| AAV CLv-D6 | 713 | U.S. Pat. No. 8,734,809 SEQ ID NO: 101 |
| AAV CLv-D7 | 714 | U.S. Pat. No. 8,734,809 SEQ ID NO: 102 |
| AAV CLv-D8 | 715 | U.S. Pat. No. 8,734,809 SEQ ID NO: 103 |
| AAV CLv-E1 | 716 | U.S. Pat. No. 8,734,809 SEQ ID NO: 87 |
| AAV CLv-R1 | 717 | U.S. Pat. No. 8,734,809 SEQ ID NO: 104 |
| AAV CLv-R2 | 718 | U.S. Pat. No. 8,734,809 SEQ ID NO: 105 |
| AAV CLv-R3 | 719 | U.S. Pat. No. 8,734,809 SEQ ID NO: 106 |
| AAV CLv-R4 | 720 | U.S. Pat. No. 8,734,809 SEQ ID NO: 107 |
| AAV CLv-R5 | 721 | U.S. Pat. No. 8,734,809 SEQ ID NO: 108 |
| AAV CLv-R6 | 722 | U.S. Pat. No. 8,734,809 SEQ ID NO: 109 |
| AAV CLv-R7 | 723 | U.S. Pat. No. 8,734,809 SEQ ID NO: 110 |
| AAV CLv-R8 | 724 | U.S. Pat. No. 8,734,809 SEQ ID NO: 111 |
| AAV CLv-R9 | 725 | U.S. Pat. No. 8,734,809 SEQ ID NO: 112 |
| AAV CLg-F1 | 726 | U.S. Pat. No. 8,734,809 SEQ ID NO: 113 |
| AAV CLg-F2 | 727 | U.S. Pat. No. 8,734,809 SEQ ID NO: 114 |
| AAV CLg-F3 | 728 | U.S. Pat. No. 8,734,809 SEQ ID NO: 115 |
| AAV CLg-F4 | 729 | U.S. Pat. No. 8,734,809 SEQ ID NO: 116 |
| AAV CLg-F5 | 730 | U.S. Pat. No. 8,734,809 SEQ ID NO: 117 |
| AAV CLg-F6 | 731 | U.S. Pat. No. 8,734,809 SEQ ID NO: 117 |
| AAV CLg-F7 | 732 | U.S. Pat. No. 8,734,809 SEQ ID NO: 118 |
| AAV CLg-F8 | 733 | U.S. Pat. No. 8,734,809 SEQ ID NO: 117 |
| AAV CSp-1 | 734 | U.S. Pat. No. 8,734,809 SEQ ID NO: 119 |
| AAV CSp-10 | 735 | U.S. Pat. No. 8,734,809 SEQ ID NO: 120 |
| AAV CSp-11 | 736 | U.S. Pat. No. 8,734,809 SEQ ID NO: 121 |
| AAV CSp-2 | 737 | U.S. Pat. No. 8,734,809 SEQ ID NO: 122 |
| AAV CSp-3 | 738 | U.S. Pat. No. 8,734,809 SEQ ID NO: 123 |
| AAV CSp-4 | 739 | U.S. Pat. No. 8,734,809 SEQ ID NO: 124 |
| AAV CSp-6 | 740 | U.S. Pat. No. 8,734,809 SEQ ID NO: 125 |
| AAV CSp-7 | 741 | U.S. Pat. No. 8,734,809 SEQ ID NO: 126 |
| AAV CSp-8 | 742 | U.S. Pat. No. 8,734,809 SEQ ID NO: 127 |
| AAV CSp-9 | 743 | U.S. Pat. No. 8,734,809 SEQ ID NO: 128 |
| AAV CHt-2 | 744 | U.S. Pat. No. 8,734,809 SEQ ID NO: 129 |
| AAV CHt-3 | 745 | U.S. Pat. No. 8,734,809 SEQ ID NO: 130 |
| AAV CKd-1 | 746 | U.S. Pat. No. 8,734,809 SEQ ID NO: 131 |
| AAV CKd-10 | 747 | U.S. Pat. No. 8,734,809 SEQ ID NO: 132 |
| AAV CKd-2 | 748 | U.S. Pat. No. 8,734,809 SEQ ID NO: 133 |
| AAV CKd-3 | 749 | U.S. Pat. No. 8,734,809 SEQ ID NO: 134 |
| AAV CKd-4 | 750 | U.S. Pat. No. 8,734,809 SEQ ID NO: 135 |
| AAV CKd-6 | 751 | U.S. Pat. No. 8,734,809 SEQ ID NO: 136 |
| AAV CKd-7 | 752 | U.S. Pat. No. 8,734,809 SEQ ID NO: 137 |
| AAV CKd-8 | 753 | U.S. Pat. No. 8,734,809 SEQ ID NO: 138 |
| AAV CLv-1 | 754 | U.S. Pat. No. 8,734,809 SEQ ID NO: 139 |
| AAV CLv-12 | 755 | U.S. Pat. No. 8,734,809 SEQ ID NO: 140 |
| AAV CLv-13 | 756 | U.S. Pat. No. 8,734,809 SEQ ID NO: 141 |
| AAV CLv-2 | 757 | U.S. Pat. No. 8,734,809 SEQ ID NO: 142 |
| AAV CLv-3 | 758 | U.S. Pat. No. 8,734,809 SEQ ID NO: 143 |
| AAV CLv-4 | 759 | U.S. Pat. No. 8,734,809 SEQ ID NO: 144 |
| AAV CLv-6 | 760 | U.S. Pat. No. 8,734,809 SEQ ID NO: 145 |
| AAV CLv-8 | 761 | U.S. Pat. No. 8,734,809 SEQ ID NO: 146 |
| AAV CKd-B1 | 762 | U.S. Pat. No. 8,734,809 SEQ ID NO: 147 |
| AAV CKd-B2 | 763 | U.S. Pat. No. 8,734,809 SEQ ID NO: 148 |
| AAV CKd-B3 | 764 | U.S. Pat. No. 8,734,809 SEQ ID NO: 149 |
| AAV CKd-B4 | 765 | U.S. Pat. No. 8,734,809 SEQ ID NO: 150 |
| AAV CKd-B5 | 766 | U.S. Pat. No. 8,734,809 SEQ ID NO: 151 |
| AAV CKd-B6 | 767 | U.S. Pat. No. 8,734,809 SEQ ID NO: 152 |
| AAV CKd-B7 | 768 | U.S. Pat. No. 8,734,809 SEQ ID NO: 153 |
| AAV CKd-B8 | 769 | U.S. Pat. No. 8,734,809 SEQ ID NO: 154 |
| AAV CKd-H1 | 770 | U.S. Pat. No. 8,734,809 SEQ ID NO: 155 |
| AAV CKd-H2 | 771 | U.S. Pat. No. 8,734,809 SEQ ID NO: 156 |
| AAV CKd-H3 | 772 | U.S. Pat. No. 8,734,809 SEQ ID NO: 157 |
| AAV CKd-H4 | 773 | U.S. Pat. No. 8,734,809 SEQ ID NO: 158 |
| AAV CKd-H5 | 774 | U.S. Pat. No. 8,734,809 SEQ ID NO: 159 |
| AAV CKd-H6 | 775 | U.S. Pat. No. 8,734,809 SEQ ID NO: 151 |
| AAV CHt-1 | 776 | U.S. Pat. No. 8,734,809 SEQ ID NO: 160 |
| AAV CHt-P2 | 777 | WO2016065001 SEQ ID NO: 1 |
| AAV CHt-P5 | 778 | WO2016065001 SEQ ID NO: 2 |
| AAV CHt-P9 | 779 | WO2016065001 SEQ ID NO: 3 |
| AAV CBr-7.1 | 780 | WO2016065001 SEQ ID NO: 4 |
| AAV CBr-7.2 | 781 | WO2016065001 SEQ ID NO: 5 |
| AAV CBr-7.3 | 782 | WO2016065001 SEQ ID NO: 6 |
| AAV CBr-7.4 | 783 | WO2016065001 SEQ ID NO: 7 |
| AAV CBr-7.5 | 784 | WO2016065001 SEQ ID NO: 8 |
| AAV CBr-7.7 | 785 | WO2016065001 SEQ ID NO: 9 |
| AAV CBr-7.8 | 786 | WO2016065001 SEQ ID NO: 10 |
| AAV CBr-7.10 | 787 | WO2016065001 SEQ ID NO: 11 |
| AAV CKd-N3 | 788 | WO2016065001 SEQ ID NO: 12 |
| AAV CKd-N4 | 789 | WO2016065001 SEQ ID NO: 13 |
| AAV CKd-N9 | 790 | WO2016065001 SEQ ID NO: 14 |
| AAV CLv-L4 | 791 | WO2016065001 SEQ ID NO: 15 |
| AAV CLv-L5 | 792 | WO2016065001 SEQ ID NO: 16 |
| AAV CLv-L6 | 793 | WO2016065001 SEQ ID NO: 17 |
| AAV CLv-K1 | 794 | WO2016065001 SEQ ID NO: 18 |
| AAV CLv-K3 | 795 | WO2016065001 SEQ ID NO: 19 |
| AAV CLv-K6 | 796 | WO2016065001 SEQ ID NO: 20 |
| AAV CLv-M1 | 797 | WO2016065001 SEQ ID NO: 21 |
| AAV CLV-M11 | 798 | WO2016065001 SEQ ID NO: 22 |
| AAV CLv-M2 | 799 | WO2016065001 SEQ ID NO: 23 |
| AAV CLv-M5 | 800 | WO2016065001 SEQ ID NO: 24 |
| AAV CLv-M6 | 801 | WO2016065001 SEQ ID NO: 25 |
| AAV CLv-M7 | 802 | WO2016065001 SEQ ID NO: 26 |
| AAV CLv-M8 | 803 | WO2016065001 SEQ ID NO: 27 |
| AAV CLv-M9 | 804 | WO2016065001 SEQ ID NO: 28 |
| AAV CHt-P1 | 805 | WO2016065001 SEQ ID NO: 29 |
| AAV CHt-P6 | 806 | WO2016065001 SEQ ID NO: 30 |
| AAV CHt-P8 | 807 | WO2016065001 SEQ ID NO: 31 |
| AAV CHt-6.1 | 808 | WO2016065001 SEQ ID NO: 32 |
| AAV CHt-6.10 | 809 | WO2016065001 SEQ ID NO: 33 |
| AAV CHt-6.5 | 810 | WO2016065001 SEQ ID NO: 34 |
| AAV CHt-6.6 | 811 | WO2016065001 SEQ ID NO: 35 |
| AAV CHt-6.7 | 812 | WO2016065001 SEQ ID NO: 36 |
| AAV CHt-6.8 | 813 | WO2016065001 SEQ ID NO: 37 |
| AAV CSp-8.10 | 814 | WO2016065001 SEQ ID NO: 38 |
| AAV CSp-8.2 | 815 | WO2016065001 SEQ ID NO: 39 |
| AAV CSp-8.4 | 816 | WO2016065001 SEQ ID NO: 40 |
| AAV CSp-8.5 | 817 | WO2016065001 SEQ ID NO: 41 |
| AAV CSp-8.6 | 818 | WO2016065001 SEQ ID NO: 42 |
| AAV CSp-8.7 | 819 | WO2016065001 SEQ ID NO: 43 |
| AAV CSp-8.8 | 820 | WO2016065001 SEQ ID NO: 44 |
| AAV CSp-8.9 | 821 | WO2016065001 SEQ ID NO: 45 |
| AAV CBr-B7.3 | 822 | WO2016065001 SEQ ID NO: 46 |
| AAV CBr-B7.4 | 823 | WO2016065001 SEQ ID NO: 47 |
| AAV3B | 824 | WO2016065001 SEQ ID NO: 48 |
| AAV4 | 825 | WO2016065001 SEQ ID NO: 49 |
| AAV5 | 826 | WO2016065001 SEQ ID NO: 50 |
| AAV CHt-P2 | 827 | WO2016065001 SEQ ID NO: 51 |
| AAV CHt-P5 | 828 | WO2016065001 SEQ ID NO: 52 |
| AAV CHt-P9 | 829 | WO2016065001 SEQ ID NO: 53 |
| AAV CBr-7.1 | 830 | WO2016065001 SEQ ID NO: 54 |
| AAV CBr-7.2 | 831 | WO2016065001 SEQ ID NO: 55 |
| AAV CBr-7.3 | 832 | WO2016065001 SEQ ID NO: 56 |
| AAV CBr-7.4 | 833 | WO2016065001 SEQ ID NO: 57 |
| AAV CBr-7.5 | 834 | WO2016065001 SEQ ID NO: 58 |
| AAV CBr-7.7 | 835 | WO2016065001 SEQ ID NO: 59 |
| AAV CBr-7.8 | 836 | WO2016065001 SEQ ID NO: 60 |
| AAV CBr-7.10 | 837 | WO2016065001 SEQ ID NO: 61 |
| AAV CKd-N3 | 838 | WO2016065001 SEQ ID NO: 62 |
| AAV CKd-N4 | 839 | WO2016065001 SEQ ID NO: 63 |
| AAV CKd-N9 | 840 | WO2016065001 SEQ ID NO: 64 |
| AAV CLv-L4 | 841 | WO2016065001 SEQ ID NO: 65 |
| AAV CLv-L5 | 842 | WO2016065001 SEQ ID NO: 66 |
| AAV CLv-L6 | 843 | WO2016065001 SEQ ID NO: 67 |
| AAV CLv-K1 | 844 | WO2016065001 SEQ ID NO: 68 |
| AAV CLv-K3 | 845 | WO2016065001 SEQ ID NO: 69 |
| AAV CLv-K6 | 846 | WO2016065001 SEQ ID NO: 70 |
| AAV CLv-M1 | 847 | WO2016065001 SEQ ID NO: 71 |
| AAV CLv-M11 | 848 | WO2016065001 SEQ ID NO: 72 |
| AAV CLv-M2 | 849 | WO2016065001 SEQ ID NO: 73 |
| AAV CLv-M5 | 850 | WO2016065001 SEQ ID NO: 74 |
| AAV CLv-M6 | 851 | WO2016065001 SEQ ID NO: 75 |
| AAV CLv-M7 | 852 | WO2016065001 SEQ ID NO: 76 |
| AAV CLv-M8 | 853 | WO2016065001 SEQ ID NO: 77 |
| AAV CLv-M9 | 854 | WO2016065001 SEQ ID NO: 78 |
| AAV CHt-P1 | 855 | WO2016065001 SEQ ID NO: 79 |
| AAV CHt-P6 | 856 | WO2016065001 SEQ ID NO: 80 |
| AAV CHt-P8 | 857 | WO2016065001 SEQ ID NO: 81 |
| AAV CHt-6.1 | 858 | WO2016065001 SEQ ID NO: 82 |
| AAV CHt-6.10 | 859 | WO2016065001 SEQ ID NO: 83 |
| AAV CHt-6.5 | 860 | WO2016065001 SEQ ID NO: 84 |
| AAV CHt-6.6 | 861 | WO2016065001 SEQ ID NO: 85 |
| AAV CHt-6.7 | 862 | WO2016065001 SEQ ID NO: 86 |
| AAV CHt-6.8 | 863 | WO2016065001 SEQ ID NO: 87 |
| AAV CSp-8.10 | 864 | WO2016065001 SEQ ID NO: 88 |
| AAV CSp-8.2 | 865 | WO2016065001 SEQ ID NO: 89 |
| AAV CSp-8.4 | 866 | WO2016065001 SEQ ID NO: 90 |
| AAV CSp-8.5 | 867 | WO2016065001 SEQ ID NO: 91 |
| AAV CSp-8.6 | 868 | WO2016065001 SEQ ID NO: 92 |
| AAV CSp-8.7 | 869 | WO2016065001 SEQ ID NO: 93 |
| AAV CSp-8.8 | 870 | WO2016065001 SEQ ID NO: 94 |
| AAV CSp-8.9 | 871 | WO2016065001 SEQ ID NO: 95 |
| AAV CBr-B7.3 | 872 | WO2016065001 SEQ ID NO: 96 |
| AAV CBr-B7.4 | 873 | WO2016065001 SEQ ID NO: 97 |
| AAV3B | 874 | WO2016065001 SEQ ID NO: 98 |
| AAV4 | 875 | WO2016065001 SEQ ID NO: 99 |
| AAV5 | 876 | WO2016065001 SEQ ID NO: 100 |
| GPV | 877 | U.S. Pat. No. 9,624,274B2 SEQ ID NO: 192 |
| B19 | 878 | U.S. Pat. No. 9,624,274B2 SEQ ID NO: 193 |
| MVM | 879 | U.S. Pat. No. 9,624,274B2 SEQ ID NO: 194 |
| FPV | 880 | U.S. Pat. No. 9,624,274B2 SEQ ID NO: 195 |
| CPV | 881 | U.S. Pat. No. 9,624,274B2 SEQ ID NO: 196 |
| AAV6 | 882 | U.S. Pat. No. 9,546,112B2 SEQ ID NO: 5 |
| AAV6 | 883 | U.S. Pat. No. 9,457,103B2 SEQ ID NO: 1 |
| AAV2 | 884 | U.S. Pat. No. 9,457,103B2 SEQ ID NO: 2 |
| ShH10 | 885 | U.S. Pat. No. 9,457,103B2 SEQ ID NO: 3 |
| ShH13 | 886 | U.S. Pat. No. 9,457,103B2 SEQ ID NO: 4 |
| ShH10 | 887 | U.S. Pat. No. 9,457,103B2 SEQ ID NO: 5 |
| ShH10 | 888 | U.S. Pat. No. 9,457,103B2 SEQ ID NO: 6 |
| ShH10 | 889 | U.S. Pat. No. 9,457,103B2 SEQ ID NO: 7 |
| ShH10 | 890 | U.S. Pat. No. 9,457,103B2 SEQ ID NO: 8 |
| ShH10 | 891 | U.S. Pat. No. 9,457,103B2 SEQ ID NO: 9 |
| rh74 | 892 | U.S. Pat. No. 9,434,928B2 SEQ ID NO: 1, US2015023924A1 SEQ |
| | | ID NO: 2 |
| rh74 | 893 | U.S. Pat. No. 9,434,928B2 SEQ ID NO: 2, US2015023924A1 SEQ |
| | | ID NO: 1 |
| AAV8 | 894 | U.S. Pat. No. 9,434,928B2 SEQ ID NO: 4 |
| rh74 | 895 | U.S. Pat. No. 9,434,928B2 SEQ ID NO: 5 |
| rh74 (RHM4-1) | 896 | US2015023924A1 SEQ ID NO: 5, US20160375110A1 |
| | | SEQ ID NO: 4 |
| rh74 (RHM15-1) | 897 | US2015023924A1 SEQ ID NO: 6, US20160375110A1 |
| | | SEQ ID NO: 5 |
| rh74 (RHM15-2) | 898 | US2015023924A1 SEQ ID NO: 7, US20160375110A1 |
| | | SEQ ID NO: 6 |
| rh74 (RHM15-3/RHM15-5) | 899 | US2015023924A1 SEQ ID NO: 8, US20160375110A1 |
| | | SEQ ID NO: 7 |
| rh74 (RHM15-4) | 900 | US2015023924A1 SEQ ID NO: 9, US20160375110A1 |
| | | SEQ ID NO: 8 |
| rh74 (RHM15-6) | 901 | US2015023924A1 SEQ ID NO: 10, US20160375110A1 |
| | | SEQ ID NO: 9 |
| rh74 (RHM4-1) | 902 | US2015023924A1 SEQ ID NO: 11 |
| rh74 (RHM15-1) | 903 | US2015023924A1 SEQ ID NO: 12 |
| rh74 (RHM15-2) | 904 | US2015023924A1 SEQ ID NO: 13 |
| rh74 (RHM15-3/RHM15-5) | 905 | US2015023924A1 SEQ ID NO: 14 |
| rh74 (RHM15-4) | 906 | US2015023924A1 SEQ ID NO: 15 |
| rh74 (RHM15-6) | 907 | US2015023924A1 SEQ ID NO: 16 |
| AAV2 (comprising lung | 908 | US20160175389A1 SEQ ID NO: 9 |
| specific polypeptide) | | |
| AAV2 (comprising lung | 909 | US20160175389A1 SEQ ID NO: 10 |
| specific polypeptide) | | |
| Anc80 | 910 | US20170051257A1 SEQ ID NO: 1 |
| Anc80 | 911 | US20170051257A1 SEQ ID NO: 2 |
| Anc81 | 912 | US20170051257A1 SEQ ID NO: 3 |
| Anc80 | 913 | US20170051257A1 SEQ ID NO: 4 |
| Anc82 | 914 | US20170051257A1 SEQ ID NO: 5 |
| Anc82 | 915 | US20170051257A1 SEQ ID NO: 6 |
| Anc83 | 916 | US20170051257A1 SEQ ID NO: 7 |
| Anc83 | 917 | US20170051257A1 SEQ ID NO: 8 |
| Anc84 | 918 | US20170051257A1 SEQ ID NO: 9 |
| Anc84 | 919 | US20170051257A1 SEQ ID NO: 10 |
| Anc94 | 920 | US20170051257A1 SEQ ID NO: 11 |
| Anc94 | 921 | US20170051257A1 SEQ ID NO: 12 |
| Anc113 | 922 | US20170051257A1 SEQ ID NO: 13 |
| Anc113 | 923 | US20170051257AI SEQ ID NO: 14 |
| Anc126 | 924 | US20170051257A1 SEQ ID NO: 15 |
| Anc126 | 925 | US20170051257A1 SEQ ID NO: 16 |
| Anc127 | 926 | US20170051257A1 SEQ ID NO: 17 |
| Anc127 | 927 | US20170051257A1 SEQ ID NO: 18 |
| Anc80L27 | 928 | US20170051257A1 SEQ ID NO: 19 |
| Anc80L59 | 929 | US20170051257A1 SEQ ID NO: 20 |
| Anc80L60 | 930 | US20170051257A1 SEQ ID NO: 21 |
| Anc80L62 | 931 | US20170051257A1 SEQ ID NO: 22 |
| Anc80L65 | 932 | US20170051257A1 SEQ ID NO: 23 |
| Anc80L33 | 933 | US20170051257A1 SEQ ID NO: 24 |
| Anc80L36 | 934 | US20170051257A1 SEQ ID NO: 25 |
| Anc80L44 | 935 | US20170051257A1 SEQ ID NO: 26 |
| Anc80L1 | 936 | US20170051257A1 SEQ ID NO: 35 |
| Anc80L1 | 937 | US20170051257A1 SEQ ID NO: 36 |
| AAV-X1 | 938 | U.S. Pat. No. 8,283,151B2 SEQ ID NO: 11 |
| AAV-X1b | 939 | U.S. Pat. No. 8,283,151B2 SEQ ID NO: 12 |
| AAV-X5 | 940 | U.S. Pat. No. 8,283,151B2 SEQ ID NO: 13 |
| AAV-XI9 | 941 | U.S. Pat. No. 8,283,151B2 SEQ ID NO: 14 |
| AAV-X21 | 942 | U.S. Pat. No. 8,283,151B2 SEQ ID NO: 15 |
| AAV-X22 | 943 | U.S. Pat. No. 8,283,151B2 SEQ ID NO: 16 |
| AAV-X23 | 944 | U.S. Pat. No. 8,283,151B2 SEQ ID NO: 17 |
| AAV-X24 | 945 | U.S. Pat. No. 8,283,151B2 SEQ ID NO: 18 |
| AAV-X25 | 946 | U.S. Pat. No. 8,283,151B2 SEQ ID NO: 19 |
| AAV-X26 | 947 | U.S. Pat. No. 8,283,151B2 SEQ ID NO: 20 |
| AAV-X1 | 948 | U.S. Pat. No. 8,283,151B2 SEQ ID NO: 21 |
| AAV-X1b | 949 | U.S. Pat. No. 8,283,151B2 SEQ ID NO: 22 |
| AAV-X5 | 950 | U.S. Pat. No. 8,283,151B2 SEQ ID NO: 23 |
| AAV-X19 | 951 | U.S. Pat. No. 8,283,151B2 SEQ ID NO: 24 |
| AAV-X21 | 952 | U.S. Pat. No. 8,283,151B2 SEQ ID NO: 25 |
| AAV-X22 | 953 | U.S. Pat. No. 8,283,151B2 SEQ ID NO: 26 |
| AAV-X23 | 954 | U.S. Pat. No. 8,283,151B2 SEQ ID NO: 27 |
| AAV-X24 | 955 | U.S. Pat. No. 8,283,151B2 SEQ ID NO: 28 |
| AAV-X25 | 956 | U.S. Pat. No. 8,283,151B2 SEQ ID NO: 29 |
| AAV-X26 | 957 | U.S. Pat. No. 8,283,151B2 SEQ ID NO: 30 |
| AAVrh8 | 958 | WO2016054554A1 SEQ ID NO: 8 |
| AAVrh8VP2FC5 | 959 | WO2016054554A1 SEQ ID NO: 9 |
| AAVrh8VP2FC44 | 960 | WO2016054554A1 SEQ ID NO: 10 |
| AAVrh8VP2ApoB100 | 961 | WO2016054554A1 SEQ ID NO: 11 |
| AAVrh8VP2RVG | 962 | WO2016054554A1 SEQ ID NO: 12 |
| AAVrh8VP2Angiopep-2 | 963 | WO2016054554A1 SEQ ID NO: 13 |
| VP2 | | |
| AAV9.47VP1.3 | 964 | WO2016054554A1 SEQ ID NO: 14 |
| AAV9.47VP2ICAMg3 | 965 | WO2016054554A1 SEQ ID NO: 15 |
| AAV9.47VP2RVG | 966 | WO2016054554A1 SEQ ID NO: 16 |
| AAV9.47VP2Angiopep-2 | 967 | WO2016054554A1 SEQ ID NO: 17 |
| AAV9.47VP2A-string | 968 | WO2016054554A1 SEQ ID NO: 18 |
| AAVrh8VP2FC5 VP2 | 969 | WO2016054554A1 SEQ ID NO: 19 |
| AAVrh8VP2FC44 VP2 | 970 | WO2016054554A1 SEQ ID NO: 20 |
| AAVrh8VP2ApoB100 VP2 | 971 | WO2016054554A1 SEQ ID NO: 21 |
| AAVrh8VP2RVG VP2 | 972 | WO2016054554A1 SEQ ID NO: 22 |
| AAVrh8VP2Angiopep-2 | 973 | WO2016054554A1 SEQ ID NO: 23 |
| VP2 | | |
| AAV9.47VP2ICAMg3 VP2 | 974 | WO2016054554A1 SEQ ID NO: 24 |
| AAV9.47VP2RVG VP2 | 975 | WO2016054554A1 SEQ ID NO: 25 |
| AAV9.47VP2 Angiopep-2 | 976 | WO2016054554A1 SEQ ID NO: 26 |
| VP2 | | |
| AAV9.47VP2A-string VP2 | 977 | WO2016054554A1 SEQ ID NO: 27 |
| rAAV-B1 | 978 | WO2016054557A1 SEQ ID NO: 1 |
| rAAV-B2 | 979 | WO2016054557A1 SEQ ID NO: 2 |
| rAAV-B3 | 980 | WO2016054557A1 SEQ ID NO: 3 |
| rAAV-B4 | 981 | WO2016054557A1 SEQ ID NO: 4 |
| rAAV-B1 | 982 | WO2016054557A1 SEQ ID NO: 5 |
| rAAV-B2 | 983 | WO2016054557A1 SEQ ID NO: 6 |
| rAAV-B3 | 984 | WO2016054557A1 SEQ ID NO: 7 |
| rAAV-B4 | 985 | WO2016054557A1 SEQ ID NO: 8 |
| rAAV-L1 | 986 | WO2016054557A1 SEQ ID NO: 9 |
| rAAV-L2 | 987 | WO2016054557A1 SEQ ID NO: 10 |
| rAAV-L3 | 988 | WO2016054557A1 SEQ ID NO: 11 |
| rAAV-L4 | 989 | WO2016054557A1 SEQ ID NO: 12 |
| rAAV-L1 | 990 | WO2016054557A1 SEQ ID NO: 13 |
| rAAV-L2 | 991 | WO2016054557A1 SEQ ID NO: 14 |
| rAAV-L3 | 992 | WO2016054557A1 SEQ ID NO: 15 |
| rAAV-L4 | 993 | WO2016054557A1 SEQ ID NO: 16 |
| AAV9 | 994 | WO2016073739A1 SEQ ID NO: 3 |
| rAAV | 995 | WO2016081811A1 SEQ ID NO: 1 |
| rAAV | 996 | WO2016081811A1 SEQ ID NO: 2 |
| rAAV | 997 | WO2016081811A1 SEQ ID NO: 3 |
| rAAV | 998 | WO2016081811A1 SEQ ID NO: 4 |
| rAAV | 999 | WO2016081811A1 SEQ ID NO: 5 |
| rAAV | 1000 | WO2016081811A1 SEQ ID NO: 6 |
| rAAV | 1001 | WO2016081811A1 SEQ ID NO: 7 |
| rAAV | 1002 | WO2016081811A1 SEQ ID NO: 8 |
| rAAV | 1003 | WO2016081811A1 SEQ ID NO: 9 |
| rAAV | 1004 | WO2016081811A1 SEQ ID NO: 10 |
| rAAV | 1005 | WO2016081811A1 SEQ ID NO: 11 |
| rAAV | 1006 | WO2016081811A1 SEQ ID NO: 12 |
| rAAV | 1007 | WO2016081811A1 SEQ ID NO: 13 |
| rAAV | 1008 | WO2016081811A1 SEQ ID NO: 14 |
| rAAV | 1009 | WO2016081811A1 SEQ ID NO: 15 |
| rAAV | 1010 | WO2016081811A1 SEQ ID NO: 16 |
| rAAV | 1011 | WO2016081811A1 SEQ ID NO: 17 |
| rAAV | 1012 | WO2016081811A1 SEQ ID NO: 18 |
| rAAV | 1013 | WO2016081811A1 SEQ ID NO: 19 |
| rAAV | 1014 | WO2016081811A1 SEQ ID NO: 20 |
| rAAV | 1015 | WO2016081811A1 SEQ ID NO: 21 |
| rAAV | 1016 | WO2016081811A1 SEQ ID NO: 22 |
| rAAV | 1017 | WO2016081811A1 SEQ ID NO: 23 |
| rAAV | 1018 | WO2016081811A1 SEQ ID NO: 24 |
| rAAV | 1019 | WO2016081811A1 SEQ ID NO: 25 |
| rAAV | 1020 | WO2016081811A1 SEQ ID NO: 26 |
| rAAV | 1021 | WO2016081811A1 SEQ ID NO: 27 |
| rAAV | 1022 | WO2016081811A1 SEQ ID NO: 28 |
| rAAV | 1023 | WO2016081811A1 SEQ ID NO: 29 |
| rAAV | 1024 | WO2016081811A1 SEQ ID NO: 30 |
| rAAV | 1025 | WO2016081811A1 SEQ ID NO: 31 |
| rAAV | 1026 | WO2016081811A1 SEQ ID NO: 32 |
| rAAV | 1027 | WO2016081811A1 SEQ ID NO: 33 |
| rAAV | 1028 | WO2016081811A1 SEQ ID NO: 34 |
| rAAV | 1029 | WO2016081811A1 SEQ ID NO: 35 |
| rAAV | 1030 | WO2016081811A1 SEQ ID NO: 36 |
| rAAV | 1031 | WO2016081811A1 SEQ ID NO: 37 |
| rAAV | 1032 | WO2016081811A1 SEQ ID NO: 38 |
| rAAV | 1033 | WO2016081811A1 SEQ ID NO: 39 |
| rAAV | 1034 | WO2016081811A1 SEQ ID NO: 40 |
| rAAV | 1035 | WO2016081811A1 SEQ ID NO: 41 |
| rAAV | 1036 | WO2016081811A1 SEQ ID NO: 42 |
| rAAV | 1037 | WO2016081811A1 SEQ ID NO: 43 |
| rAAV | 1038 | WO2016081811A1 SEQ ID NO: 44 |
| rAAV | 1039 | WO2016081811A1 SEQ ID NO: 45 |
| rAAV | 1040 | WO2016081811A1 SEQ ID NO: 46 |
| rAAV | 1041 | WO2016081811A1 SEQ ID NO: 47 |
| rAAV | 1042 | WO2016081811A1 SEQ ID NO: 48 |
| rAAV | 1043 | WO2016081811A1 SEQ ID NO: 49 |
| rAAV | 1044 | WO2016081811A1 SEQ ID NO: 50 |
| rAAV | 1045 | WO2016081811A1 SEQ ID NO: 51 |
| rAAV | 1046 | WO2016081811A1 SEQ ID NO: 52 |
| rAAV | 1047 | WO2016081811A1 SEQ ID NO: 53 |
| rAAV | 1048 | WO2016081811A1 SEQ ID NO: 54 |
| rAAV | 1049 | WO2016081811A1 SEQ ID NO: 55 |
| rAAV | 1050 | WO2016081811A1 SEQ ID NO: 56 |
| rAAV | 1051 | WO2016081811A1 SEQ ID NO: 57 |
| rAAV | 1052 | WO2016081811A1 SEQ ID NO: 58 |
| rAAV | 1053 | WO2016081811A1 SEQ ID NO: 59 |
| rAAV | 1054 | WO2016081811A1 SEQ ID NO: 60 |
| rAAV | 1055 | WO2016081811A1 SEQ ID NO: 61 |
| rAAV | 1056 | WO2016081811A1 SEQ ID NO: 62 |
| rAAV | 1057 | WO2016081811A1 SEQ ID NO: 63 |
| rAAV | 1058 | WO2016081811A1 SEQ ID NO: 64 |
| rAAV | 1059 | WO2016081811A1 SEQ ID NO: 65 |
| rAAV | 1060 | WO2016081811A1 SEQ ID NO: 66 |
| rAAV | 1061 | WO2016081811A1 SEQ ID NO: 67 |
| rAAV | 1062 | WO2016081811A1 SEQ ID NO: 68 |
| rAAV | 1063 | WO2016081811A1 SEQ ID NO: 69 |
| rAAV | 1064 | WO2016081811A1 SEQ ID NO: 70 |
| rAAV | 1065 | WO2016081811A1 SEQ ID NO: 71 |
| rAAV | 1066 | WO2016081811A1 SEQ ID NO: 72 |
| rAAV | 1067 | WO2016081811A1 SEQ ID NO: 73 |
| rAAV | 1068 | WO2016081811A1 SEQ ID NO: 74 |
| rAAV | 1069 | WO2016081811A1 SEQ ID NO: 75 |
| rAAV | 1070 | WO2016081811A1 SEQ ID NO: 76 |
| rAAV | 1071 | WO2016081811A1 SEQ ID NO: 77 |
| rAAV | 1072 | WO2016081811A1 SEQ ID NO: 78 |
| rAAV | 1073 | WO2016081811A1 SEQ ID NO: 79 |
| rAAV | 1074 | WO2016081811A1 SEQ ID NO: 80 |
| rAAV | 1075 | WO2016081811A1 SEQ ID NO: 81 |
| rAAV | 1076 | WO2016081811A1 SEQ ID NO: 82 |
| rAAV | 1077 | WO2016081811A1 SEQ ID NO: 83 |
| rAAV | 1078 | WO2016081811A1 SEQ ID NO: 84 |
| rAAV | 1079 | WO2016081811A1 SEQ ID NO: 85 |
| rAAV | 1080 | WO2016081811A1 SEQ ID NO: 86 |
| rAAV | 1081 | WO2016081811A1 SEQ ID NO: 87 |
| rAAV | 1082 | WO2016081811A1 SEQ ID NO: 88 |
| rAAV | 1083 | WO2016081811A1 SEQ ID NO: 89 |
| rAAV | 1084 | WO2016081811A1 SEQ ID NO: 90 |
| rAAV | 1085 | WO2016081811A1 SEQ ID NO: 91 |
| rAAV | 1086 | WO2016081811A1 SEQ ID NO: 92 |
| rAAV | 1087 | WO2016081811A1 SEQ ID NO: 93 |
| rAAV | 1088 | WO2016081811A1 SEQ ID NO: 94 |
| rAAV | 1089 | WO2016081811A1 SEQ ID NO: 95 |
| rAAV | 1090 | WO2016081811A1 SEQ ID NO: 96 |
| rAAV | 1091 | WO2016081811A1 SEQ ID NO: 97 |
| rAAV | 1092 | WO2016081811A1 SEQ ID NO: 98 |
| rAAV | 1093 | WO2016081811A1 SEQ ID NO: 99 |
| rAAV | 1094 | WO2016081811A1 SEQ ID NO: 100 |
| rAAV | 1095 | WO2016081811A1 SEQ ID NO: 101 |
| rAAV | 1096 | WO2016081811A1 SEQ ID NO: 102 |
| rAAV | 1097 | WO2016081811A1 SEQ ID NO: 103 |
| rAAV | 1098 | WO2016081811A1 SEQ ID NO: 104 |
| rAAV | 1099 | WO2016081811A1 SEQ ID NO: 105 |
| rAAV | 1100 | WO2016081811A1 SEQ ID NO: 106 |
| rAAV | 1101 | WO2016081811A1 SEQ ID NO: 107 |
| rAAV | 1102 | WO2016081811A1 SEQ ID NO: 108 |
| rAAV | 1103 | WO2016081811A1 SEQ ID NO: 109 |
| rAAV | 1104 | WO2016081811A1 SEQ ID NO: 110 |
| rAAV | 1105 | WO2016081811A1 SEQ ID NO: 111 |
| rAAV | 1106 | WO2016081811A1 SEQ ID NO: 112 |
| rAAV | 1107 | WO2016081811A1 SEQ ID NO: 113 |
| rAAV | 1108 | WO2016081811A1 SEQ ID NO: 114 |
| rAAV | 1109 | WO2016081811A1 SEQ ID NO: 115 |
| rAAV | 1110 | WO2016081811A1 SEQ ID NO: 116 |
| rAAV | 1111 | WO2016081811A1 SEQ ID NO: 117 |
| rAAV | 1112 | WO2016081811A1 SEQ ID NO: 118 |
| rAAV | 1113 | WO2016081811A1 SEQ ID NO: 119 |
| rAAV | 1114 | WO2016081811A1 SEQ ID NO: 120 |
| rAAV | 1115 | WO2016081811A1 SEQ ID NO: 121 |
| rAAV | 1116 | WO2016081811A1 SEQ ID NO: 122 |
| rAAV | 1117 | WO2016081811A1 SEQ ID NO: 123 |
| rAAV | 1118 | WO2016081811A1 SEQ ID NO: 124 |
| rAAV | 1119 | WO2016081811A1 SEQ ID NO: 125 |
| rAAV | 1120 | WO2016081811A1 SEQ ID NO: 126 |
| rAAV | 1121 | WO2016081811A1 SEQ ID NO: 127 |
| rAAV | 1122 | WO2016081811A1 SEQ ID NO: 128 |
| AAV8 E532K | 1123 | WO2016081811A1 SEQ ID NO: 133 |
| AAV8 E532K | 1124 | WO2016081811A1 SEQ ID NO: 134 |
| rAAV | 1125 | WO2016115382A1 SEQ ID NO: 2 |
| rAAV | 1126 | WO2016115382A1 SEQ ID NO: 3 |
| rAAV | 1127 | WO2016115382A1 SEQ ID NO: 4 |
| rAAV | 1128 | WO2016115382A1 SEQ ID NO: 5 |
| rAAV | 1129 | WO2016115382A1 SEQ ID NO: 6 |
| rAAV | 1130 | WO2016115382A1 SEQ ID NO: 7 |
| rAAV | 1131 | WO2016115382A1 SEQ ID NO: 8 |
| rAAV | 1132 | WO2016115382A1 SEQ ID NO: 9 |
| rAAV | 1133 | WO2016115382A1 SEQ ID NO: 10 |
| rAAV | 1134 | WO2016115382A1 SEQ ID NO: 11 |
| rAAV | 1135 | WO2016115382A1 SEQ ID NO: 12 |
| rAAV | 1136 | WO2016115382A1 SEQ ID NO: 13 |
| rAAV | 1137 | WO2016115382A1 SEQ ID NO: 14 |
| rAAV4 | 1138 | WO2016115382A1 SEQ ID NO: 15 |
| rAAV4 | 1139 | WO2016115382A1 SEQ ID NO: 16 |
| rAAV4 | 1140 | WO2016115382A1 SEQ ID NO: 17 |
| rAAV4 | 1141 | WO2016115382A1 SEQ ID NO: 18 |
| rAAV4 | 1142 | WO2016115382A1 SEQ ID NO: 19 |
| rAAV4 | 1143 | WO2016115382A1 SEQ ID NO: 20 |
| rAAV4 | 1144 | WO2016115382A1 SEQ ID NO: 21 |
| AAV11 | 1145 | WO2016115382A1 SEQ ID NO: 22 |
| AAV12 | 1146 | WO2016115382A1 SEQ ID NO: 23 |
| rh32 | 1147 | WO2016115382A1 SEQ ID NO: 25 |
| rh33 | 1148 | WO2016115382A1 SEQ ID NO: 26 |
| rh34 | 1149 | WO2016115382A1 SEQ ID NO: 27 |
| rAAV4 | 1150 | WO2016115382A1 SEQ ID NO: 28 |
| rAAV4 | 1151 | WO2016115382A1 SEQ ID NO: 29 |
| rAAV4 | 1152 | WO2016115382A1 SEQ ID NO: 30 |
| rAAV4 | 1153 | WO2016115382A1 SEQ ID NO: 31 |
| rAAV4 | 1154 | WO2016115382A1 SEQ ID NO: 32 |
| rAAV4 | 1155 | WO2016115382A1 SEQ ID NO: 33 |
| AAV2/8 | 1156 | WO2016131981A1 SEQ ID NO: 47 |
| AAV2/8 | 1157 | WO2016131981A1 SEQ ID NO: 48 |
| ancestral AAV | 1158 | WO2016154344A1 SEQ ID NO: 7 |
| ancestral AAV variant C4 | 1159 | WO2016154344A1 SEQ ID NO: 13 |
| ancestral AAV variant C7 | 1160 | WO2016154344A1 SEQ ID NO: 14 |
| ancestral AAV variant G4 | 1161 | WO2016154344A1 SEQ ID NO: 15 |
| consensus amino acid | 1162 | WO2016154344A1 SEQ ID NO: 16 |
| sequence of ancestral AAV | | |
| variants, C4, C7 and G4 | | |
| consensus amino acid | 1163 | WO2016154344A1 SEQ ID NO: 17 |
| sequence of ancestral AAV | | |
| variants, C4 and C7 | | |
| AAV8 (with a AAV2 | 1164 | WO2016150403A1 SEQ ID NO: 13 |
| phospholipase domain) | | |
| AAV VR-942n | 1165 | US20160289275A1 SEQ ID NO: 10 |
| AAV5-A (M569V) | 1166 | US20160289275A1 SEQ ID NO: 13 |
| AAV5-A (M569V) | 1167 | US20160289275A1 SEQ ID NO: 14 |
| AAV5-A (Y585V) | 1168 | US20160289275A1 SEQ ID NO: 16 |
| AAV5-A (Y585V) | 1169 | US20160289275A1 SEQ ID NO: 17 |
| AAV5-A (L587T) | 1170 | US20160289275A1 SEQ ID NO: 19 |
| AAV5-A (L587T) | 1171 | US20160289275A1 SEQ ID NO: 20 |
| AAV5-A (Y585V/L587T) | 1172 | US20160289275A1 SEQ ID NO: 22 |
| AAV5-A (Y585V/L587T) | 1173 | US20160289275A1 SEQ ID NO: 23 |
| AAV5-B (D652A) | 1174 | US20160289275A1 SEQ ID NO: 25 |
| AAV5-B (D652A) | 1175 | US20160289275A1 SEQ ID NO: 26 |
| AAV5-B (T362M) | 1176 | US20160289275A1 SEQ ID NO: 28 |
| AAV5-B (T362M) | 1177 | US20160289275A1 SEQ ID NO: 29 |
| AAV5-B (Q359D) | 1178 | US20160289275A1 SEQ ID NO: 31 |
| AAV5-B (Q359D) | 1179 | US20160289275A1 SEQ ID NO: 32 |
| AAV5-B (E350Q) | 1180 | US20160289275A1 SEQ ID NO: 34 |
| AAV5-B (E350Q) | 1181 | US20160289275A1 SEQ ID NO: 35 |
| AAV5-B (P533S) | 1182 | US20160289275A1 SEQ ID NO: 37 |
| AAV5-B (P533S) | 1183 | US20160289275A1 SEQ ID NO: 38 |
| AAV5-B (P533G) | 1184 | US20160289275A1 SEQ ID NO: 40 |
| AAV5-B (P533G) | 1185 | US20160289275A1 SEQ ID NO: 41 |
| AAV5-mutation in loop VII | 1186 | US20160289275A1 SEQ ID NO: 43 |
| AAVS-mutation in loop VII | 1187 | US20160289275A1 SEQ ID NO: 44 |
| AAV8 | 1188 | US20160289275A1 SEQ ID NO: 47 |
| Mut A (LK03/AAV8) | 1189 | WO2016181123A1 SEQ ID NO: 1 |
| Mut B (LK03/AAV5) | 1190 | WO2016181123A1 SEQ ID NO: 2 |
| Mut C (AAV8/AAV3B) | 1191 | WO2016181123A1 SEQ ID NO: 3 |
| Mut D (AAV5/AAV3B) | 1192 | WO2016181123A1 SEQ ID NO: 4 |
| Mut E (AAV8/AAV3B) | 1193 | WO2016181123A1 SEQ ID NO: 5 |
| Mut F (AAV3B/AAV8) | 1194 | WO2016181123A1 SEQ ID NO: 6 |
| AAV44.9 | 1195 | WO2016183297A1 SEQ ID NO: 4 |
| AAV44.9 | 1196 | WO2016183297A1 SEQ ID NO: 5 |
| AAVrh8 | 1197 | WO2016183297A1 SEQ ID NO: 6 |
| AAV44.9 (S470N) | 1198 | WO2016183297A1 SEQ ID NO: 9 |
| rh74 VP1 | 1199 | US20160375110A1 SEQ ID NO: 1 |
| AAV-LK03 (L125I) | 1200 | WO2017015102A1 SEQ ID NO: 5 |
| AAV3B (S663V + T492V) | 1201 | WO2017015102A1 SEQ ID NO: 6 |
| Anc80 | 1202 | WO2017019994A2 SEQ ID NO: 1 |
| Anc80 | 1203 | WO2017019994A2 SEQ ID NO: 2 |
| Anc81 | 1204 | WO2017019994A2 SEQ ID NO: 3 |
| Anc81 | 1205 | WO2017019994A2 SEQ ID NO: 4 |
| Anc82 | 1206 | WO2017019994A2 SEQ ID NO: 5 |
| Anc82 | 1207 | WO2017019994A2 SEQ ID NO: 6 |
| Anc83 | 1208 | WO2017019994A2 SEQ ID NO: 7 |
| Anc83 | 1209 | WO2017019994A2 SEQ ID NO: 8 |
| Anc84 | 1210 | WO2017019994A2 SEQ ID NO: 9 |
| Anc84 | 1211 | WO2017019994A2 SEQ ID NO: 10 |
| Anc94 | 1212 | WO2017019994A2 SEQ ID NO: 11 |
| Anc94 | 1213 | WO2017019994A2 SEQ ID NO: 12 |
| Anc113 | 1214 | WO2017019994A2 SEQ ID NO: 13 |
| Anc113 | 1215 | WO2017019994A2 SEQ ID NO: 14 |
| Anc126 | 1216 | WO2017019994A2 SEQ ID NO: 15 |
| Anc126 | 1217 | WO2017019994A2 SEQ ID NO: 16 |
| Anc127 | 1218 | WO2017019994A2 SEQ ID NO: 17 |
| Anc127 | 1219 | WO2017019994A2 SEQ ID NO: 18 |
| Anc80L27 | 1220 | WO2017019994A2 SEQ ID NO: 19 |
| Anc80L59 | 1221 | WO2017019994A2 SEQ ID NO: 20 |
| Anc80L60 | 1222 | WO2017019994A2 SEQ ID NO: 21 |
| Anc80L62 | 1223 | WO2017019994A2 SEQ ID NO: 22 |
| Anc80L65 | 1224 | WO2017019994A2 SEQ ID NO: 23 |
| Anc80L33 | 1225 | WO2017019994A2 SEQ ID NO: 24 |
| Anc80L36 | 1226 | WO2017019994A2 SEQ ID NO: 25 |
| Anc80L44 | 1227 | WO2017019994A2 SEQ ID NO: 26 |
| Anc80L1 | 1228 | WO2017019994A2 SEQ ID NO: 35 |
| Anc80L1 | 1229 | WO2017019994A2 SEQ ID NO: 36 |
| AAVrh10 | 1230 | WO2017019994A2 SEQ ID NO: 41 |
| Anc110 | 1231 | WO2017019994A2 SEQ ID NO: 42 |
| Anc110 | 1232 | WO2017019994A2 SEQ ID NO: 43 |
| AAVrh32.33 | 1233 | WO2017019994A2 SEQ ID NO: 45 |
| AAVrh74 | 1234 | WO2017049031A1 SEQ ID NO: 1 |
| AAV2 | 1235 | WO2017053629A2 SEQ ID NO: 49 |
| AAV2 | 1236 | WO2017053629A2 SEQ ID NO: 50 |
| AAV2 | 1237 | WO2017053629A2 SEQ ID NO: 82 |
| Parvo-like virus | 1238 | WO2017070476A2 SEQ ID NO: 1 |
| Parvo-like virus | 1239 | WO2017070476A2 SEQ ID NO: 2 |
| Parvo-like virus | 1240 | WO2017070476A2 SEQ ID NO: 3 |
| Parvo-like virus | 1241 | WO2017070476A2 SEQ ID NO: 4 |
| Parvo-like virus | 1242 | WO2017070476A2 SEQ ID NO: 5 |
| Parvo-like virus | 1243 | WO2017070476A2 SEQ ID NO: 6 |
| AAVrh.10 | 1244 | WO2017070516A1 SEQ ID NO: 7 |
| AAVrh.10 | 1245 | WO2017070516A1 SEQ ID NO: 14 |
| AAV2tYF | 1246 | WO2017070491A1 SEQ ID NO: 1 |
| AAV-SPK | 1247 | WO2017075619A1 SEQ ID NO: 28 |
| AAV2.5 | 1248 | US20170128528A1 SEQ ID NO: 13 |
| AAV1.1 | 1249 | US20170128528A1 SEQ ID NO: 15 |
| AAV6.1 | 1250 | US20170128528A1 SEQ ID NO: 17 |
| AAV6.3.1 | 1251 | US20170128528A1 SEQ ID NO: 18 |
| AAV218 | 1252 | US20170128528A1 SEQ ID NO: 28 |
| AAV218 | 1253 | US20170128528A1 SEQ ID NO: 29 |
| ttAAV | 1254 | US20170128528A1 SEQ ID NO: 30 |
| ttAAV-S312N | 1255 | US20170128528A1 SEQ ID NO: 32 |
| ttAAV-S312N | 1256 | US20170128528A1 SEQ ID NO: 33 |
| AAV6 (Y705, Y731, and | 1257 | WO2016134337A1 SEQ ID NO: 24 |
| T492) | | |
| AAV2 | 1258 | WO2016134375A1 SEQ ID NO: 9 |
| AAV2 | 1259 | WO2016134375A1 SEQ ID NO: 10 |

In some embodiments, the AAV serotype may be, or may have a sequence as described in International Patent Publication WO2015038958, the contents of which are herein incorporated by reference in their entirety, such as, but not limited to, AAV9 (SEQ ID NO: 2 and 11 of WO2015038958 or SEQ ID NO: 135 and 136 respectively herein), PHP.B (SEQ ID NO: 8 and 9 of WO2015038958, herein SEQ ID NO: 3 and 4), G2B-13 (SEQ ID NO: 12 of WO2015038958, herein SEQ ID NO: 5), G2B-26 (SEQ ID NO: 13 of WO2015038958, herein SEQ TD NO: 3), TH1.1-32 (SEQ ID NO: 14 of WO2015038958 herein SEQ ID NO: 6), TH1.1-35 (SEQ ID NO: 15 of WO2015038958, herein SEQ ID NO: 7) or variants thereof. Further, any of the targeting peptides or amino acid inserts described in WO2015038958, may be inserted into any parent AAV serotype, such as, but not limited to, AAV9 (SEQ ID NO: 135 for the DNA sequence and SEQ ID NO: 136 for the amino acid sequence). In some embodiments, the amino acid insert is inserted between amino acids 586-592 of the parent AAV (e.g., AAV9). Ln another embodiment, the amino acid insert is inserted between amino acids 588-589 of the parent AAV sequence. The amino acid insert may be, but is not limited to, any of the following amino acid sequences, TLAVPFK (SEQ ID NO: 1 of WO2015038958; herein SEQ ID NO: 1260), KFPVALT (SEQ ID NO: 3 of WO2015038958; herein SEQ ID NO: 1261), LAVPFK (SEQ ID NO: 31 of WO2015038958; herein SEQ ID NO: 1262), AVPFK (SEQ ID NO: 32 of WO2015038958; herein SEQ ID NO: 1263), VPFK (SEQ ID NO: 33 of WO2015038958; herein SEQ ID NO: 1264), TLAVPF (SEQ ID NO: 34 of WO2015038958; herein SEQ ID NO: 1265), TLAVP (SEQ ID NO: 35 of WO2015038958; herein SEQ ID NO: 1266), TLAV (SEQ ID NO: 36 of WO2015038958; herein SEQ ID NO: 1267), SVSKPFL (SEQ ID NO: 28 of WO2015038958; herein SEQ ID NO: 1268), FTLTTPK (SEQ ID NO: 29 of WO2015038958; herein SEQ ID NO: 1269), MNATKNV (SEQ ID NO: 30 of WO2015038958; herein SEQ ID NO: 1270), QSSQTPR. (SEQ ID NO: 54 of WO2015038958; herein SEQ ID NO: 1271), ILGTGTS (SEQ ID NO: 55 of WO2015038958; herein SEQ ID NO: 1272), TRTNPEA (SEQ ID NO: 56 of WO2015038958; herein SEQ ID NO: 1273), NGGTSSS (SEQ ID NO: 58 of WO2015038958; herein SEQ ID NO: 1274), or YTLSQGW (SEQ ID NO: 60 of WO2015038958; herein SEQ NO: 1275). Non-limiting examples of nucleotide sequences that may encode the amino acid inserts include the following, AAGTTTCCTGTGGCGTTGACT (for SEQ ID NO: 3 of WO2015038958; herein SEQ ID NO: 1276), ACTTTGGCGGTGCCTTTTAAG (SEQ ID NO: 24 and 49 of WO2015038958; herein SEQ ID NO: 1277), AGTGTGAGTAAGCCTTTTTTG (SEQ ID NO: 25 of WO2015038958; herein SEQ ID NO: 1278), TTTACGTTGACGACGCCTAAG (SEQ ID NO: 26 of WO2015038958; herein SEQ ID NO: 1279), ATGAATGCTACGAAGAATGTG (SEQ ID NO: 27 of WO2015038958; herein SEQ ID NO: 1280), CAGTCGTCGCAGACGCCTAGG (SEQ NO: 48 of WO2015038958; herein SEQ ID NO: 1281), ATTCTGGGGACTGGTACTTCG (SEQ ID NO: 50 and 52 of WO2015038958; herein SEQ ID NO: 1282), ACGCGGACTAATCCTGAGGCT (SEQ ID NO: 51 of WO2015038958; herein SEQ ID NO: 1283), AATGGGGGGACTAGTAGTTCT (SEQ ID NO: 53 of WO201.5038958; herein SEQ ID NO: 1284), or TATACTITGTCGCAGGGTTGG (SEQ ID NO: 59 of WO2015038958; herein SEQ ID NO: 1285).

In some embodiments, the AAV serotype may be, or may have a sequence as described in International Patent Publication WO2017100671, the contents of which are herein incorporated by reference in their entirety, such as, but not limited to, AAV9 (SEQ ID NO: 45 of WO2017100671, herein SEQ ID NO: 9), PHP.N (SEQ ID NO: 46 of WO2017100671, herein SEQ ID NO: 2), PHP.S (SEQ ID NO: 47 of WO201.7100671, herein SEQ ID NO: 8), or variants thereof. Further, any of the targeting peptides or amino acid inserts described in WO2017100671 may be inserted into any parent AAV serotype, such as, but not limited to, AAV9 (SEQ ID NO: 9 or SEQ ID NO: 131). In some embodiments, the amino acid insert is inserted between amino acids 586-592 of the parent AAV (e.g., AAV9). In another embodiment, the amino acid insert is inserted between amino acids 588589 of the parent AAV sequence. The amino acid insert may be, but is not limited to, any of the following amino acid sequences, AQTLAVPFKAQ (SEQ ID NO: 1 of WO20171.00671; herein SEQ ID NO: 1286), AQSVSKPFLAQ (SEQ ID NO: 2 of WO2017100671; herein SEQ ID NO: 1287), AQFTLTTPKAQ (SEQ ID NO: 3 in the sequence listing of WO2017100671; herein SEQ ID NO: 1288), DGTLAVPFKAQ (SEQ ID NO: 4 in the sequence listing of WO2017100671; herein SEQ ID NO: 1289), ESTLAVPFKAQ (SEQ ID NO: 5 of WO20171.00671; herein SEQ ID NO: 1290), GGTLAVPFKAQ (SEQ ID NO: 6 of WO2017100671, herein SEQ ID NO: 1291), AQTLATPFKAQ (SEQ ID NO: 7 and 33 of WO2017100671; herein SEQ ID NO: 1292), ATTLATPFKAQ (SEQ ID NO: 8 of WO2017100671; herein SEQ ID NO: 1293), DGTLATPFKAQ (SEQ ID NO: 9 of WO2017100671; herein SEQ ID NO: 1294), GGTLATPFKAQ (SEQ NO: 10 of 902017100671; herein SEQ ID NO: 1295), SGSLAVPFKAQ (SEQ ID NO: 11 of WO2017100671; herein SEQ ID NO: 1296), AQTLAQPFKAQ (SEQ ID NO: 12 of WO2017100671; herein SEQ ID NO: 1297), AQTLQQPFKAQ (SEQ ID NO: 13 of WO2017100671; herein SEQ ID NO: 1298), AQTLSNPFKAQ (SEQ ID NO: 14 of WO201.7100671; herein SEQ ID NO: 1299), AQTLAVPFSNP (SEQ ID NO: 15 of WO2017100671; herein SEQ ID NO: 1300), QGTLAVPFKAQ (SEQ ID NO: 16 of WO2017100671; herein SEQ ID NO: 1301), NQTLAVPFKAQ (SEQ ID NO: 17 of 902017100671; herein SEQ ID NO: 1302), EGSLAVPFKAQ (SEQ ID NO: 18 of WO2017100671; herein SEQ ID NO: 1303), SGNLAVPFKAQ (SEQ ID NO: 19 of WO2017100671; herein SEQ ID NO: 1304), EGTLAVPFKAQ (SEQ ID NO: 20 of WO2017100671; herein SEQ ID NO: 1305), DSTLAVPFKAQ (SEQ ID NO: 21 in Table 1 of WO2017100671; herein SEQ ID NO: 1306), AVTLAVPFKAQ (SEQ ID NO: 22 of WO2017100671; herein SEQ ID NO: 1307), AQILSTRFKAQ (SEQ NO: 23 of WO2017100671; herein SEQ ID NO: 1308), AQTLPQPFKAQ (SEQ ID NO: 24 and 32 of WO2017100671; herein SEQ ID NO: 1309), AQTLSQPFKAQ (SEQ ID NO: 25 of WO2017100671; herein SEQ ID NO: 1310), AQTLQLPFKAQ (SEQ ID NO: 26 of WO2017100671; herein SEQ ID NO: 1311), AQTLTMPFKAQ (SEQ ID NO: 27, and 34 of WO2017100671 and SEQ ID NO: 35 in the sequence listing of WO20171.00671; herein SEQ TD NO: 1312), AQTLTTPFKAQ (SEQ TD NO: 28 of WO2017100671; herein SEQ ID NO: 1313), AQYTLSQGWAQ (SEQ ID NO: 29 of WO2017100671; herein SEQ ID NO: 1314), AQMNATKNVAQ (SEQ ID NO: 30 of WO2017100671; herein SEQ ID NO: 1315), AQVSGGEIIISAQ (SEQ ID NO: 31 of WO2017100671; herein SEQ ID NO: 1316), AQTLTAPFKAQ (SEQ ID NO: 35 in Table 1 of WO2017100671; herein SEQ ID NO: 1317), AQTLSKPFKAQ (SEQ ID NO: 36 of WO2017100671; herein SEQ ID NO: 1318), QAVRTSL (SEQ ID NO: 37 of WO2017100671; herein SEQ ID NO: 1319), YTLSQGW (SEQ ID NO: 38 of WO2017100671; herein SEQ ID NO: 1275), LAKERLS (SEQ ID NO: 39 of WO2017100671; herein SEQ ID NO: 1320), TLAVPFK (SEQ ID NO: 40 in the sequence listing of WO2017100671; herein SEQ ID NO: 1260), SVSKPFL (SEQ ID NO: 41 of WO2017100671; herein SEQ ID NO: 1268), FTLTTPK (SEQ ID NO: 42 of WO2017100671; herein SEQ ID NO: 1269), MNSTKNV (SEQ ID NO: 43 of WO2017100671; herein SEQ ID NO: 1321), VSGGHHS (SEQ ID NO: 44 of WO2017100671; herein SEQ ID NO: 1322), SAQTLAVPFKAQAQ (SEQ ID NO: 48 of WO2017100671; herein SEQ ID NO: 1323), SXXXLAVPIFKAQAQ (SEQ ID NO: 49 of WO2017100671 wherein X may be any amino acid; herein SEQ ID NO: 1324), SAQXXXVPFKAQAQ (SEQ ID NO: 50 of WO2017100671 wherein X may be any amino acid; herein SEQ ID NO: 1325), SAQTLXXXTKAQAQ (SEQ ID NO: 51 of WO2017100671 wherein X may be any amino acid; herein SEQ ID NO: 1326), SAQTLAVXXXAQAQ (SEQ ID NO: 52 of WO2017100671 wherein X may be any amino acid; herein SEQ ID NO: 1327), SAQTLAVPFXXXAQ (SEQ ID NO: 53 of WO2017100671 wherein X may be any amino acid; herein SEQ ID NO: 1328), TNHQSAQ (SEQ ID NO: 65 of WO2017100671; herein SEQ ID NO: 1329), AQAQTGW (SEQ ID NO: 66 of WO2017100671; herein SEQ ID NO: 1330), DGTLATPFK (SEQ ID NO: 67 of WO2017100671; herein SEQ ID NO: 1331), DGTLATPFKXX (SEQ ID NO: 68 of WO2017100671 wherein X may be any amino acid; herein SEQ ID NO: 1332), LAVPFKAQ (SEQ ID NO: 80 of WO2017100671; herein SEQ NO: 1333), VPFKAQ (SEQ ID NO: 81 of WO2017100671; herein SEQ ID NO: 1334), FKAQ (SEQ ID NO: 82 of WO20171.00671; herein SEQ ID NO: 1335), AQTLAV (SEQ TD NO: 83 of WO2017100671; herein SEQ ID NO: 1336), AQTLAVPF (SEQ ID NO: 84 of WO2017100671; herein SEQ ID NO: 1337), QAVR (SEQ ID NO: 85 of WO2017100671; herein SEQ ID NO: 1338), AVRT (SEQ ID NO: 86 of WO2017100671; herein SEQ ID NO: 1339), VRTS (SEQ ID NO: 87 of WO2017100671; herein SEQ ID NO: 1340), RTSL (SEQ ID NO: 88 of WO2017100671; herein SEQ ID NO: 1341), QAVRT (SEQ ID NO: 89 of WO2017100671; herein SEQ ID NO: 1342), AVRTS (SEQ ID NO: 90 of WO2017100671; herein SEQ ID NO: 1343), VRTSL (SEQ ID NO: 91 of WO2017100671; herein SEQ ID NO: 1344), QAVRTS (SEQ ID NO: 92 of WO2017100671; herein SEQ ID NO: 1345), or AVRTSL (SEQ ID NO: 93 of WO2017100671; herein SEQ ID NO: 1346).

Non-limiting examples of nucleotide sequences that may encode the amino acid inserts include the following, GATGGGACTTTGGCGGTGCCTTTTAAGGCACAG (SEQ ID NO: 54 of WO2017100671; herein SEQ ID NO: 1347), GATGGGACGTTGGCGGTGCCTTTTAAGGCACAG (SEQ ID NO: 55 of WO2017100671; herein SEQ ID NO: 1348), CAGGCGGTTAGGACGICTFTG (SEQ ID NO: 56 of WO2017100671; herein SEQ ID NO: 1349), CAGGTCTTCACGGACTCAGACTATCAG (SEQ ID NO: 57 and 78 of WO2017100671; herein SEE ID NO: 1350), CAAGTAAAACCTCTACAAATGTGGTAAAATCG (SEQ ID NO: 58 of WO2017100671; herein SEQ ID NO: 1351), ACTCATCGACCAATACTTGTACTATCTCTCTAGAAC (SEQ ID NO: 59 of WO2017100671; herein SEQ ID NO: 1352), GGAAGTATTCCTTGGTTTTGAACCCA (SEQ ID NO: 60 of WO20171.00671; herein SEQ Ili NO: 1353), GGTCGCGGTTCTTGTTTGTGGAT (SEQ ID NO: 61 of WO2017100671; herein SEQ ID NO: 1354), CGACCTTGAAGCGCATGAACTCCT (SEQ ID NO: 62 of WO2017100671; herein SEQ ID NO: 1355), GTATTCCTTGGTTITGAACCCAACCGGTCTGCGCCTGTGCMNNMNNMNNMNNMNN MNNMNNTTGGGCACTCTGGTGGTTTGTC (SEQ ID NO: 63 of WO2017100671 wherein N may be A, C, T, or G; herein SEQ ID NO: 1356), GTATTCCTTGGTTTTGAACCCAACCGGTCTGCGCNINNIVFNMYINNAAAAGGCACCGCC AAAGTTTG (SEQ ID NO: 69 of WO2017100671 wherein N may be A, C, T, or G; herein SEQ ID NO: 1357), GTATTCCTTGGTTTTGAACCCAACCGGTCTGCGCCTGTGCMNNMNNMNNAAAAGGCACCGCC AAAGTTTGGGCACT (SEQ ID NO: 70 of WO2017100671 wherein N may be A, C, T, or G; herein SEQ ID NO: 1358), GTATTCCTTGGTTTTGAACCCAACCGGTCTGCGCCTGTGCCTTAAAMNNMNNMNNC AAAGTTTGGGCACTCTGGTGG (SEQ ID NO: 71 of WO2017100671 wherein N may be A, C, T, or G; herein SEQ ID NO: 1359), GTATTCCTTGGTTTTGAACCCAACCGGTCTGCGCCTGTGCCTTAAAAGGCACMNNM NNMNNTTGGGCACTCTGGTGGTTTGTG (SEQ ID NO: 72 of W(0D2017100671 wherein N may be A, C, T, or G; herein SEQ ID NO: 1360), ACTTTGGCGGTGCCTTTTAAG (SEQ ID NO: 74 of WO2017100671; herein SEQ ID NO: 1277), AGTGTGAGTAAGCCTTTTTTG (SEQ ID NO: 75 of WO2017100671; herein SEQ ID NO: 1278), TTTACGTTGACGACGCCTAAG (SEQ ID NO: 76 of WO2017100671; herein SEQ ID NO: 1279), TATACTTTGTCGCAGTGTTGG (SEQ ID NO: 77 of WO2017100671; herein SEQ ID NO: 1285), or CTTGCGAAGGAGCGGCTTTCG (SEQ ID NO: 79 of WO2017100671; herein SEQ ID NO: 1361).

In some embodiments, the AAV serotype may be, or may have a sequence as described in U.S. Pat. No. 9,624,274, the contents of which are herein incorporated by reference in their entirety, such as, but not limited to, AAV1 (SEQ ID NO: 181 of US9624274), AAV6 (SEQ ID NO: 182 of US9624274), AAV2 (SEQ ID NO: 183 of US9624274), AAV3b (SEQ ID NO: 184 of US9624274), AAV7 (SEQ ID NO: 185 of US9624274), AAV8 (SEQ ID NO: 186 of US9624274), AAV10 (SEQ ID NO: 187 of U.S. Pat. No. 9,624,274), AAV4 (SEQ ID NO: 188 of U.S. Pat. No. 9,624,274), AAV11 (SEQ ID NO: 189 of U.S. Pat. No. 9,624,274), bAAV (SEQ ID NO: 190 of US9624274), AAV5 (SEQ ID NO: 191 of U.S. Pat. No. 9,624,274), GPV (SEQ ID NO: 192 of US9624274; herein SEQ ID NO: 992), 1319 (SEQ ID NO: 193 of 059624274; herein SEQ ID NO: 993), MVM (SEQ ID NO: 194 of U.S. Pat. No. 9,624,274; herein SEQ ID NO: 994), FPV (SEQ ID NO: 195 of U.S. Pat. No. 9,624,274; herein SEQ ID NO: 995), CPV (SEQ ID NO: 196 of US9624274; herein SEQ ID NO: 996) or variants thereof. Further, any of the structural protein inserts described in U.S. Pat. No. 9,624,274, may be inserted into, but not limited to, 1-453 and 1-587 of any parent AAV serotype, such as, but not limited to, AAV2 (SEQ ID NO: 183 of US9624274), The amino acid insert may be, but is not limited to, any of the following amino acid sequences, VNLTWSRASG (SEQ ID NO: 50 of U.S. Pat. No. 9,624,274; herein SEQ ID NO: 1362), EFCINIERGYWVCGD (SEQ ID NO:55 of 059624274; herein SEQ ID NO: 1363), EDGQVMDVDLS (SEQ ID NO: 85 of 1159624274; herein SEQ ID NO: 1364), EKQRNGTLT (SEQ ID NO: 86 of U.S. Pat. No. 9,624,274; herein SEQ ID NO: 1365), TYQCRVTEIPEILPRALMR (SEQ ID NO: 87 of 059624274; herein SEQ ID NO: 1366), RHSTTQPRKTKGSG (SEQ ID NO: 88 of U.S. Pat. No. 9,624,274; herein SEQ ID NO: 1367), DSNPRGVSAYLSR (SEQ ID NO: 89 of U.S. Pat. No. 9,624,274; herein SEQ ID NO: 1368), TITCLWDLAPSK (SEQ ID NO: 90 of U.S. Pat. No. 9,624,274; herein SEQ ID NO: 1369), KTKGSGFFVF (SEQ ID NO: 91 of US9624274; herein SEQ ID NO: 1370), THPHLPRALMRS (SEQ ID NO: 92 of 059624274; herein SEQ ID NO: 1371), GETYQCRVTHPHLPRALMRSTTK (SEQ ID NO: 93 of U.S. Pat. No. 9,624,274; herein SEQ ID NO: 1372), LPRALMRS (SEQ ID NO: 94 of U.S. Pat. No. 9,624,274; herein SEQ ID NO: 1373), INFIRGYWV (SEQ ID NO: 95 of US9624274; herein SEQ ID NO: 1374), CDAGSVRTNAPD (SEQ ID NO: 60 of U.S. Pat. No. 9,624,274; herein SEQ ID NO: 1375), AKAVSNLTESRSESLQS (SEQ ID NO: 96 of U.S. Pat. No. 9,624,274; herein SEQ ID NO: 1376), SLTGDEFKKVLET (SEQ ID NO: 97 of U.S. Pat. No. 9,624,274; herein SEQ ID NO: 1377), REAVAYRFEED (SEQ ID NO: 98 of U.S. Pat. No. 9,624,274; herein SEQ ID NO: 1378), INPETITLDG (SEQ ID NO: 99 of U.S. Pat. No. 9,624,274; herein SEQ ID NO: 1379), DISVTGAPVITATYL (SEQ ID NO: 100 of U.S. Pat. No. 9,624,274; herein SEQ ID NO: 1380), DISVTGAPVITA (SEQ ID NO: 101 of US9624274; herein SEQ ID NO: 1381), PKIVSNLIESSSESVQS (SEQ ID NO: 102 of U.S. Pat. No. 9,624,274; herein SEQ ID NO: 1382), SLMGDEFKAVLET (SEQ ID NO: 103 of U.S. Pat. No. 9,624,274; herein SEQ ID NO: 1383), QHSVAYTFEED (SEQ ID NO: 104 of U.S. Pat. No. 9,624,274; herein SEQ ID NO: 1384), INPEIITRDG (SEQ ID NO: 105 of U.S. Pat. No. 9,624,274; herein SEQ ID NO: 1385), DISLTGDPVITASYL (SEQ ID NO: 106 of US9624274; herein SEQ ID NO: 1386), DISLIGDPVITA (SEQ ID NO: 107 of US9624274; herein SEQ ID NO: 1387), DQSIDFEIDSA (SEQ ID NO: 108 of U.S. Pat. No. 9,624,274; herein SEQ ID NO: 1388), KNVSEDLPLPTFSPTLLGDS (SEQ ID NO: 109 of U.S. Pat. No. 9,624,274; herein SEQ ID NO: 1389), KNVSEDLPLPT (SEQ ID NO: 110 of U.S. Pat. No. 9,624,274; herein SEQ ID NO: 1390), CDSGRVRTDAPD (SEQ ID NO: 111 of U.S. Pat. No. 9,624,274; herein SEQ ID NO: 1391), FPEFILLVDFLQSLS (SEQ ID NO: 112 of U.S. Pat. No. 9,624,274; herein SEQ ID NO: 1392), DAEFREIDSG (SEQ ID NO: 65 of US9624274; herein SEQ ID NO: 1393), HYAAAQWDFGNTMCQL (SEQ ID NO: 113 of U.S. Pat. No. 9,624,274; herein SEQ ID NO: 1394), YAAQWDFGNIMCQ (SEQ ID NO: 114 of U.S. Pat. No. 9,624,274; herein SEQ ID NO: 1395), RSQKEGLHYT (SEQ ID NO: 115 of U.S. Pat. No. 9,624,274; herein SEQ ID NO: 1396), SSRTPSDKPVAHWANPQAE (SEQ ID NO: 116 of US9624274; herein SEQ ID NO: 1397), SRTPSDKPVAIIWANP (SEQ ID NO: 117 of 059624274; herein SEQ ID NO: 1398), SSRIPSDKP (SEQ ID NO: 118 of U.S. Pat. No. 9,624,274; herein SEQ ID NO: 1399), NADGNVDYHMNSVP (SEQ ID NO: 119 of 059624274; herein SEQ ID NO: 1400), DGNVDYIEMNSV (SEQ ID NO: 120 of U.S. Pat. No. 9,624,274; herein SEQ ID NO: 1401), RSFKEFLQSSLRALRQ (SEQ ID NO: 121 of US9624274; herein SEQ ID NO: 1402); FKEFLQSSLRA (SEQ ID NO: 122 of US9624274; herein SEQ ID NO: 1403), or QTYPWAPQWGPD (SEQ ID NO: 123 of U.S. Pat. No. 9,624,274; herein SEQ ID NO: 1404).

In some embodiments, the AAV serotype may be, or may have a sequence as described in U.S. Pat. No. 9,475,845, the contents of which are herein incorporated by reference in their entirety, such as, but not limited to, AAV capsid proteins comprising modification of one or more amino acids at amino acid positions 585 to 590 of the native AAV2 capsid protein. Further the modification may result in, but not limited to, the amino acid sequence RGNRQA. (SEQ ID NO: 3 of U.S. Pat. No. 9,475,845; herein SEQ ID NO: 1405), SSSTDP (SEQ ID NO: 4 of U.S. Pat. No. 9,475,845; herein SEQ ID NO: 1406), SSNTAP (SEQ ID NO: 5 of U.S. Pat. No. 9,475,845; herein SEQ ID NO: 1407), SNSNLP (SEQ ID NO: 6 of 059475845; herein SEQ ID NO: 1408), SSTTAP (SEQ ID NO: 7 of U.S. Pat. No. 9,475,845; herein SEQ ID NO: 1409), AANTAA (SEQ ID NO: 8 of US9475845; herein SEQ ID NO: 1410), QQNTAP (SEQ ID NO: 9 of 059475845; herein SEQ ID NO: 1411); SAQAQA (SEQ ID NO: 10 of U.S. Pat. No. 9,475,845; herein SEQ ID NO: 1412), QANTGP (SEQ ID NO: 11 of 059475845; herein SEQ ID NO: 1413), NATTAP (SEQ ID NO: 12 of US9475845; herein SEQ ID NO: 1414), SSTAGP (SEQ ID NO: 13 and 20 of US9475845; herein SEQ ID NO: 1415), QQNTAA (SEQ ID NO: 14 of US9475845; herein SEQ ID NO: 1416), PSTAGP (SEQ ID NO: 15 of U.S. Pat. No. 9,475,845; herein SEQ ID NO: 1417), NQNTAP (SEQ ID NO: 16 of U.S. Pat. No. 9,475,845; herein SEQ ID NO: 1418), QAANAP (SEQ ID NO: 17 of U.S. Pat. No. 9,475,845; herein SEQ ID NO: 1419), SIVGLP (SEQ ID NO: 18 of U.S. Pat. No. 9,475,845; herein SEQ ID NO: 1420), AASTAA (SEQ ID NO: 19, and 27 of U.S. Pat. No. 9,475,845; herein SEQ ID NO: 1421), SQNTTA (SEQ ID NO: 21 of U.S. Pat. No. 9,475,845; herein SEQ ID NO: 1422), QQDTAP (SEQ ID NO: 22 of US9475845; herein SEQ ID NO: 1423), QTNTGP (SEQ ID NO: 23 of U.S. Pat. No. 9,475,845; herein SEQ ID NO: 1424), QTNGAP (SEQ ID NO: 24 of US9475845; herein SEQ ID NO: 1425), QQNAAP (SEQ ID NO: 25 of U.S. Pat. No. 9,475,845; herein SEQ ID NO: 1426), or AANTQA (SEQ ID NO: 26 of 059475845; herein SEQ ID NO: 1427). In some embodiments, the amino acid modification is a substitution at amino acid positions 262 through 265 in the native AAV2 capsid protein or the corresponding position in the capsid protein of another AAV with a targeting sequence. The targeting sequence may be, but is not limited to, any of the amino acid sequences, NGRAHA (SEQ ID NO: 38 of U.S. Pat. No. 9,475,845; herein SEQ ID NO: 1428), QPEHSST (SEQ ID NO: 39 and 50 of US9475845, herein SEQ ID NO: 1429), VNTANST (SEQ ID NO: 40 of 059475845; herein SEQ ID NO: 1430), HGPMQKS (SEQ ID NO: 41 of U.S. Pat. No. 9,475,845; herein SEQ ID NO: 1431), PITKPPLA (SEQ ID NO: 42 of 059475845; herein SEQ ID NO: 1432), IKNNEMW (SEQ ID NO: 43 of U.S. Pat. No. 9,475,845; herein SEQ ID NO: 1433), RNLDTPM (SEQ ID NO: 44 of U.S. Pat. No. 9,475,845; herein SEQ ID NO: 1434), VDSHRQS (SEQ ID NO: 45 of US9475845; herein SEQ ID NO: 1435), YDSKTKT (SEQ ID NO: 46 of 0S9475845; herein SEQ ID NO: 1436), SQLPHQK (SEQ ID NO: 47 of US9475845; herein SEQ ID NO: 1437), STMQQNT (SEQ ID NO: 48 of 0S9475845; herein SEQ ID NO: 1438), TERYMTQ (SEQ ID NO: 49 of US9475845; herein SEQ ID NO: 1439), DASLSTS (SEQ ID NO: 51 of U.S. Pat. No. 9,475,845; herein SEQ ID NO: 1440), DLPNKKT (SEQ ID NO: 52 of 059475845; herein SEQ ID NO: 1441), DLTAARL (SEQ ID NO: 53 of U.S. Pat. No. 9,475,845; herein SEQ ID NO: 1442), EPHQFNY (SEQ ID NO: 54 of 059475845; herein SEQ ID NO: 1443), EPQSNHT (SEQ ID NO: 55 of US9475845; herein SEQ ID NO: 1444), MSSWPSQ (SEQ ID NO: 56 of U.S. Pat. No. 9,475,845; herein SEQ ID NO: 1445), NPKHNAT (SEQ ID NO: 57 of U.S. Pat. No. 9,475,845; herein SEQ ID NO: 1446), PDGIVIRTT (SEQ ED NO: 58 of US9475845; herein SEQ ID NO: 1447), PNNNKTT (SEQ ID NO: 59 of U.S. Pat. No. 9,475,845; herein SEQ ID NO: 1448), QSTTHDS (SEQ ID NO: 60 of U.S. Pat. No. 9,475,845; herein SEQ ID NO: 1449), TGSKQKQ (SEQ ID NO: 61 of U.S. Pat. No. 9,475,845; herein SEQ ID NO: 1450), SLKHQAL (SEQ ID NO: 62 of U.S. Pat. No. 9,475,845; herein SEQ ID NO: 1451), SPIDGEQ (SEQ ID NO: 63 of US9475845; herein SEQ ID NO: 1452), WIFPWIQL (SEQ ID NO: 64 and 112 of U.S. Pat. No. 9,475,845; herein SEQ ID NO: 1453), CDCRGDCFC (SEQ ID NO: 65 of U.S. Pat. No. 9,475,845; herein SEQ ID NO: 1454), CNGRC (SEQ ID NO: 66 of U.S. Pat. No. 9,475,845; herein SEQ ID NO: 1455), CPRECES (SEQ ID NO: 67 of U.S. Pat. No. 9,475,845; herein SEQ ID NO: 1456), CTTHWGFTLC (SEQ ID NO: 68 and 123 of U.S. Pat. No. 9,475,845; herein SEQ ID NO: 1457), CGRRAGGSC (SEQ ID NO: 69 of U.S. Pat. No. 9,475,845; herein SEQ ID NO: 1458), CKGGRAKDC (SEQ ID NO: 70 of US9475845; herein SEQ ID NO: 1459), CVPELGHEC (SEQ ID NO: 71 and 115 of U.S. Pat. No. 9,475,845; herein SEQ ID NO: 1460), CRRETAWAK (SEQ ID NO: 72 of US9475845; herein SEQ ID NO: 1461), VSWFSHRYSPFAVS (SEQ ID NO: 73 of U.S. Pat. No. 9,475,845; herein SEQ ID NO: 1462), GYRDGYAGPILYN (SEQ ID NO: 74 of U.S. Pat. No. 9,475,845; herein SEQ ID NO: 1463), XXXYXXX (SEQ ID NO: 75 of U.S. Pat. No. 9,475,845; herein SEQ 11) NO: 1464), YXNW (SEQ ID NO: 76 of U.S. Pat. No. 9,475,845; herein SEQ ID NO: 1465), RPLPPLP (SEQ ID NO: 77 of U.S. Pat. No. 9,475,845; herein SEQ ID NO: 1466), APPLPPR (SEQ ID NO: 78 of U.S. Pat. No. 9,475,845; herein SEQ ID NO: 1467), DWYPYPYASGS (SEQ ID NO: 79 of U.S. Pat. No. 9,475,845; herein SEQ ID NO: 1468), MYWYPY (SEQ 1D NO: 80 of U.S. Pat. No. 9,475,845; herein SEQ ID NO: 1469), DITWDQLWDLMK (SEQ ID NO: 81 of U.S. Pat. No. 9,475,845; herein SEQ ID NO: 1470), CWDDXWLC (SEQ ID NO: 82 of US9475845; herein SEQ ID NO: 1471), EWCEYLGGYLRCYA (SEQ ID NO: 83 of US9475845; herein SEQ ID NO: 1472), YXCXXGPXTWXCXP (SEQ ID NO: 84 of US9475845; herein SEQ NO: 1473), IEGPTLRQWLAARA (SEQ ID NO: 85 of U.S. Pat. No. 9,475,845; herein SEQ ID NO: 1474), LWXXX (SEQ ID NO: 86 of U.S. Pat. No. 9,475,845; herein SEQ ID NO: 1475), XFXXYDAT (SEQ ID NO: 87 of U.S. Pat. No. 9,475,845; herein SEQ ID NO: 1476), SSIISHFRWGLCD (SEQ ID NO: 88 of U.S. Pat. No. 9,475,845; herein SEQ ID NO: 1477), MSRPACPPNDKYE (SEQ ID NO: 89 of US9475845; herein SEQ ID NO: 1478), CLRSGRGC (SEQ ID NO: 90 of U.S. Pat. No. 9,475,845; herein SEQ ID NO: 1479), CHWMFSPWC (SEQ ID NO: 91 of US9475845; herein SEQ ID NO: 1480), WXXF (SEQ ID NO: 92 of U.S. Pat. No. 9,475,845; herein SEQ ID NO: 1481), CSSRLDAC (SEQ ID NO: 93 of U.S. Pat. No. 9,475,845; herein SEQ ID NO: 1482), CLPVASC (SEQ ID NO: 94 of US9475845; herein. SEQ ID NO: 1483); CGFECVRQCPERC (SEQ ID NO: 95 of US9475845; herein SEQ ID NO: 1484), CVALCREACGEGC (SEQ ID NO: 96 of US9475845; herein SEQ ID NO: 1485), SWCEPGWCR (SEQ ID NO: 97 of US9475845; herein SEQ ID NO: 1486), YSGKWGW (SEQ ID NO: 98 of U.S. Pat. No. 9,475,845, herein SEQ ID NO: 1487), GLSGGRS (SEQ ID NO: 99 of U.S. Pat. No. 9,475,845; herein SEQ ID NO: 1488), LMLPRAD (SEQ ID NO: 100 of U.S. Pat. No. 9,475,845; herein SEQ ID NO: 1489), CSCFRDVCC (SEQ ID NO: 101 of U.S. Pat. No. 9,475,845; herein SEQ ID NO: 1490), CRDVVSVIC (SEQ ID NO: 102 of U.S. Pat. No. 9,475,845; herein SEQ ID NO: 1491), MARSGL (SEQ ID NO: 103 of US9475845; herein SEQ ID NO: 1492), MARAKE (SEQ ID NO: 104 of U.S. Pat. No. 9,475,845, herein SEQ ID NO: 1493), MSRTMS (SEQ ID NO: 105 of U.S. Pat. No. 9,475,845; herein SEQ ID NO: 1494), KCCYSL (SEQ ID NO: 106 of U.S. Pat. No. 9,475,845; herein SEQ ID NO: 1495), MYWGDSHWLQYWYE (SEQ ID NO: 107 of U.S. Pat. No. 9,475,845; herein SEQ ID NO: 1496), MQLPLAT (SEQ ID NO: 108 of US9475845; herein SEQ ID NO: 1497), EWLS (SEQ ID NO: 109 of U.S. Pat. No. 9,475,845; herein SEQ ID NO: 1498), SKEW (SEQ ID NO: 110 of U.S. Pat. No. 9,475,845, herein SEQ ID NO: 1499), 71NYL (SEQ ID NO: 111 of U.S. Pat. No. 9,475,845; herein SEQ ID NO: 1500), WDLAWMFRLPVG (SEQ ID NO: 113 of 059475845; herein SEQ ID NO: 1501), CTVALPGGYVRVC (SEQ ID NO: 114 of U.S. Pat. No. 9,475,845; herein SEQ ID NO: 1502), CVAYCIEHHCWTC (SEQ ID NO: 116 of U.S. Pat. No. 9,475,845; herein SEQ ID NO: 1503), CVFAHNYDYLVC (SEQ ID NO: 117 of 059475845; herein SEQ ID NO: 1504), CVFTSNYAFC (SEQ ID NO: 118 of U.S. Pat. No. 9,475,845; herein SEQ ID NO: 1505), VHSPNKK (SEQ ID NO: 119 of U.S. Pat. No. 9,475,845; herein SEQ ID NO: 1506), CRGDGWC (SEQ ID NO: 120 of U.S. Pat. No. 9,475,845; herein SEQ ID NO: 1507), XRGCDX (SEQ ID NO: 121 of U.S. Pat. No. 9,475,845; herein SEQ ID NO: 1508), PXXX (SEQ ID NO: 122 of U.S. Pat. No. 9,475,845; herein SEQ ID NO: 1509), SGKGPRQITAL (SEQ ID NO: 124 of U.S. Pat. No. 9,475,845; herein SEQ ID NO: 1510), AAAAAAAAAXXXXX (SEQ ID NO: 125 of 059475845; herein SEQ ID NO: 1511), VYMSPF (SEQ ID NO: 126 of U.S. Pat. No. 9,475,845, herein SEQ ID NO: 1512), ATWLPPR (SEQ ID NO: 127 of U.S. Pat. No. 9,475,845; herein SEQ ID NO: 1513), HTMYYHHVQHHL (SEQ ID NO: 128 of U.S. Pat. No. 9,475,845; herein SEQ ID NO: 1514), SEVGCRAGPLQWLCEKYFG (SEQ ID NO: 129 of U.S. Pat. No. 9,475,845, herein SEQ ID NO: 1515), CGLLPVGRPDRNVWRWLC (SEQ ID NO: 130 of U.S. Pat. No. 9,475,845, herein SEQ ID NO: 1516), CKGQCDRFKGLPWEC (SEQ ID NO: 131 of U.S. Pat. No. 9,475,845; herein SEQ ID NO: 1517), SGRSA (SEQ ID NO: 132 of 059475845; herein SEQ ID NO: 1518), WGFP (SEQ ID NO: 133 of U.S. Pat. No. 9,475,845; herein SEQ ID NO: 1519), AEPMPHSLNFSQYLWYT (SEQ ID NO: 134 of U.S. Pat. No. 9,475,845; herein SEQ ID NO: 1520), WAYXSP (SEQ ID NO: 135 of U.S. Pat. No. 9,475,845, herein SEQ ID NO: 1521), IELLQAR (SEQ ID NO: 136 of U.S. Pat. No. 9,475,845; herein SEQ ID NO: 1522), AYTKCSRQWRTCNITTIL (SEQ ID NO: 137 of US9475845; herein SEQ ID NO: 1523), PQNSKIPGPTELDPH (SEQ ID NO: 138 of U.S. Pat. No. 9,475,845; herein SEQ ID NO: 1524), SMEPALPDWWWKMFK (SEQ ID NO: 139 of US9475845; herein SEQ ID NO: 1525), ANTPCGPYTHDCPVKR (SEQ ID NO: 140 of U.S. Pat. No. 9,475,845; herein SEQ ID NO: 1526), TACHQEIVRIVIVRP (SEQ ID NO: 141 of 059475845; herein SEQ ID NO: 1527), VPWMEPAYQRFL (SEQ ID NO: 142 of 059475845; herein SEQ ID NO: 1528), DPRATPGS (SEQ ID NO: 143 of U.S. Pat. No. 9,475,845; herein SEQ ID NO: 1529), FRPNRAQDYNTN (SEQ ID NO: 144 of 059475845; herein SEQ ID NO: 1530), CIKNSYLMC (SEQ ID NO: 145 of 059475845; herein SEQ ID NO: 1531), CXXTXXXGXGC (SEQ ID NO: 146 of U.S. Pat. No. 9,475,845; herein SEQ ID NO: 1532), CPIEDRPMC (SEQ ID NO: 147 of US9475845; herein SEQ ID NO: 1533), HEWSYLAPYPWF (SEQ ID NO: 148 of U.S. Pat. No. 9,475,845; herein SEQ ID NO: 1534), MCPKHPLGC (SEQ ID NO: 149 of U.S. Pat. No. 9,475,845; herein SEQ ID NO: 1535), RMWPSSTVNLSAGRR (SEQ ID NO: 150 of U.S. Pat. No. 9,475,845; herein SEQ ID NO: 1536), SAKTAVSQRVWLPSHRGGEP (SEQ ID NO: 151 of U.S. Pat. No. 9,475,845; herein SEQ ID NO: 1537), KSREFIVNNSACPSKRITAAL (SEQ ID NO: 152 of U.S. Pat. No. 9,475,845; herein SEQ ID NO: 1538), EGFR (SEQ ID NO: 153 of U.S. Pat. No. 9,475,845; herein SEQ ID NO. 1539), AGLGVR (SEQ ID NO: 154 of 059475845; herein SEQ ID NO: 1540), GTRQGHTMRLGVSDG (SEQ ID NO: 155 of U.S. Pat. No. 9,475,845; herein SEQ ID NO: 1541), IAGLATPGWSHWLAL (SEQ ID NO: 156 of 059475845; herein SEQ ID NO: 1542), SMSIARL (SEQ ID NO: 157 of 059475845; herein SEQ ID NO: 1543), HTFEPGV (SEQ ID NO: 158 of 059475845; herein SEQ ID NO: 1544), NTSLKRISNKRIRRK (SEQ TD NO: 159 of U.S. Pat. No. 9,475,845; herein SEQ ID NO: 1545), LRIKRKRRKRKKTRK (SEQ ID NO: 160 of U.S. Pat. No. 9,475,845; herein SEQ ID NO: 1546), GGG, GFS, LWS, EGG, LIN, LSP, LBS, AGG, GRR, GGH and GIV.

In some embodiments, the AAV serotype may be, or may have a sequence as described in United States Publication No. US 20160369298, the contents of which are herein incorporated by reference in their entirety, such as, but not limited to, site-specific mutated capsid protein of AAV2 (SEQ ID NO: 97 of US 20160369298; herein SEQ ID NO: 1547) or variants thereof, wherein the specific site is at least one site selected from sites R447, 6453, S578, N587, N587+1, 5662 of VP1 or fragment thereof.

Further, any of the mutated sequences described in US 20160369298, may be or may have, but not limited to, any of the following sequences SDSGASN (SEQ ID NO: 1 and SEQ TD NO: 231 of 0520160369298; herein SEQ ID NO: 1548), SDSGASN (SEQ ID NO: 2 of US20160369298; herein SEQ ID NO: 1549), SDSGASN (SEQ ID NO: 3 of US20160369298; herein SEQ ID NO: 1550), SRSGASN (SEQ ID NO: 4 of US20160369298; herein SEQ ID NO: 1551), SKSGASN (SEQ ID NO: 5 of 0520160369298; herein SEQ ID NO: 1552), SDSGASN (SEQ ID NO: 6 of US20160369298; herein SEQ ID NO: 1553), SDSGASN (SEQ ID NO: 7 of US20160369298; herein SEQ ID NO: 1554), SASGASN (SEQ ID NO: 8, 175, and 221 of US20160369298; herein SEQ ID NO: 1555), SESGTSN (SEQ ID NO: 9 of US20160369298; herein SEQ ID NO: 1556), STTGGSN (SEQ ID NO: 10 of 0520160369298; herein SEQ TD NO: 1557), SSAGSTN (SEQ ID NO: 11 of US20160369298; herein SEC) ID NO: 1558), NNDSQA (SEQ ID NO: 12 of US20160369298; herein SEQ ID NO: 1559), NNRNQA (SEQ ID NO: 13 of US20160369298; herein SEQ 1D NO: 1560), NNNKQA (SEQ ID NO: 14 of US20160369298; herein SEQ ID NO: 1561), NAKRQA (SEQ ID NO: 15 of US20160369298; herein SEQ ID NO: 1562), NDEHQA (SEQ ID NO: 16 of US20160369298; herein SEQ ID NO: 1563), NTSQKA (SEQ ID NO: 17 of US20160369298; herein SEQ ID NO: 1564), YYLSRTNTPSGTDTQSRLVFSQAGA (SEC) ID NO: 18 of US20160369298; herein SEQ ID NO: 1565), YYLSRTNTDSGTETQSGLDFSQAGA (SEQ ID NO: 19 of US20160369298; herein SEQ ID NO: 1566), YYLSRTNTESGIPTQSALEFSQAGA. (SEQ ID NO: 20 of US20160369298; herein SEQ ID NO: 1567), YYLSRTNTHSGTHTQSPLHFSQAGA (SEQ ID NO: 21 of US20160369298; herein SEQ ID NO: 1568), YYLSRINTSSGTITISHUTSQAGA (SEQ ID NO: 22 of US20160369298; herein SEC) ID NO: 1569), YYLSRTNTRSGIMTKSSINIFSQAGA (SEQ ID NO: 23 of US20160369298; herein SEQ TD NO: 1570), YYLSRTNTKSGRKTLSNLSFSQACiA (SEQ ID NO: 24 of US20160369298; herein SEQ ID NO: 1571), YYLSRTNDGSGPVTPSKLRFSQRGA (SEQ ID NO: 25 of US20160369298; herein SEQ ID NO: 1572), YYLSRTNAASGHATHSDLKFSQPGA (SEQ ID NO: 26 of US20160369298; herein SEQ ID NO: 1573), YYLSWINGQAGSLIMSELGFSQVGA (SEQ ID NO: 27 of US20160369298; herein SEQ ID NO: 1574), YYLSRTNSTGGNQTTSQLLFSQLSA (SEQ ID NO: 28 of US20160369298; herein SEQ ID NO: 1575), YFLSRTNNNTGLNTNSTLNFSQGRA (SEQ ID NO: 29 of US20160369298; herein SEQ ID NO: 1576), SKTGADNNNSEYSWIG (SEQ ID NO: 30 of US20160369298; herein SEQ ID NO: 1577), SKTDADNNNSEYSWTG (SEQ ID NO: 31 of US20160369298; herein SEC) ID NO: 1578), SKTEADNNNSEYSWTG (SEC) ID NO: 32 of US20160369298; herein SEQ ID NO: 1579), SKTPADNNNSEYSWTG (SEQ ID NO: 33 of US20160369298; herein SEQ ID NO: 1580), SKTHADNNNSEYSNITIG (SEQ ID NO: 34 of US20160369298; herein SEQ ID NO: 1581), SKTQADNNNSEYSWTG (SEQ ID NO: 35 of US20160369298; herein SEQ ID NO: 1582), SKTIADNNNSEYSWTG (SEQ ID NO: 36 of US20160369298; herein SEC) ID NO: 1583), SKTMADNNNSEYSWTG (SEQ ID NO: 37 of US20160369298; herein SEQ ID NO: 1584), SKTRADNNNSEYSWTG (SEQ ID NO: 38 of US20160369298; herein SEQ ID NO: 1585), SKTNADNNNSEYSWIG (SEQ ID NO: 39 of US20160369298; herein SEQ ID NO: 1586), SKTVGRNNNSEYSWTG (SEQ ID NO: 40 of US20160369298; herein SEQ ID NO: 1587), SKTADRNNNSEYSWTG (SEQ ID NO: 41 of US20160369298; herein SEQ ID NO: 1588), SKKLSQNNNSKYSWQG (SEQ ID NO: 42 of US20160369298; herein SEQ ID NO: 1589), SKPTTGNNNSDYSWPG (SEQ TD NO: 43 of US20160369298; herein SEQ ID NO: 1590), STQKNENNNSNYSWPG (SEQ ID NO: 44 of US20160369298; herein SEQ ID NO: 1591), IIKDDEGKF (SEQ ID NO: 45 of US20160369298; herein SEQ ID NO: 1592), fiKDDNRKF (SEQ ID NO: 46 of US20160369298; herein SEQ ID NO: 1593), LIKDDTNKF (SEQ ID NO: 47 of US20160369298; herein SEQ ID NO: 1594), HEDSDKNF (SEQ ID NO: 48 of US20160369298; herein SEQ ID NO: 1595), EIRDGADSF (SEQ ID NO: 49 of US20160369298; herein SEQ ID NO: 1596), HGDNKSRF (SEQ ID NO: 50 of US20160369298; herein SEQ ID NO: 1597), KQGSEKTNVDFEEV (SEQ ID NO: 51 of US20160369298; herein SEQ ID NO: 1598), KQGSEKTNVDSEEV (SEQ ID NO: 52 of US20160369298; herein SEQ ID NO: 1599), KQGSEKTNVDVEEV (SEQ ID NO: 53 of US20160369298; herein SEQ ID NO: 1600), KQGSDKTNVDDAGV (SEQ ID NO: 54 of US20160369298; herein SEQ ID NO: 1601), KQGSSKTNVDPREV (SEQ ID NO: 55 of US20160369298; herein SEQ ID NO: 1602), KQGSRKTNVIDIIKQV (SEQ ID NO: 56 of US20160369298; herein SEQ ID NO: 1603), KQGSKGGNVDTNRV (SEQ ID NO: 57 of US20160369298; herein SEQ ID NO: 1604), KQGSGEANVDNGDV (SEQ ID NO: 58 of US20160369298; herein SEQ ID NO: 1605), KQDAAADNIDYINIV (SEQ ID NO: 59 of US20160369298; herein SEQ ID NO: 1606), KQSGTRSNAAASSV (SEQ ID NO: 60 of US20160369298; herein SEQ ID NO: 1607), KENTNTNDTELTNV (SEQ ID NO: 61 of US20160369298; herein SEQ ID NO: 1608), QRGNNVAATADVNT (SEQ ID NO: 62 of US20160369298; herein SEQ ID NO: 1609), QRGNNEAATADVNT (SEQ ID NO: 63 of US20160369298; herein SEQ ID NO: 1610), QRGNNPAATADVNT (SEQ ID NO: 64 of US20160369298; herein SEQ ID NO: 1611), QRGNNEIAATADVNT (SEQ ID NO: 65 of US20160369298; herein SEQ ID NO: 1612), QEENNIAATPGVNT (SEQ ID NO: 66 of US20160369298; herein SEQ ID NO: 1613), QPPNNMAATHEVNT (SEQ ID NO: 67 of US20160369298; herein SEQ ID NO: 1614), QIIHNNSAATTIVNT (SEQ ID NO: 68 of US20160369298; herein SEQ ID NO: 1615), QTTNNRAAFNMVET (SEQ ID NO: 69 of US20160369298; herein SEQ ID NO: 1616), QKKNNNAASKKVAT (SEQ ID NO: 70 of US20160369298; herein SEQ ID NO: 1617), QGGNNKAADDAVKT (SEQ ID NO: 71 of US20160369298; herein SEQ ID NO: 1618), QAAKGGAADDAVKT (SEQ ID NO: 72 of US20160369298; herein SEQ ID NO: 1619), QDDRAAAANESVDT (SEQ ID NO: 73 of US20160369298; herein SEQ ID NO: 1620), QQQIIDDAAYQRVIIT (SEQ ID NO: 74 of US20160369298; herein SEQ ID NO: 1621), QSSSSLAAVSTVQT (SEQ ID NO: 75 of US20160369298; herein SEQ ID NO: 1622), QNNQTTAAIRNVTT (SEQ ID NO: 76 of US20160369298; herein SEQ ID NO: 1623), NYNKKSDNVDFT (SEQ ID NO: 77 of US20160369298; herein SEQ ID NO: 1624), NYNKKSENVDFT (SEQ ID NO: 78 of US20160369298; herein SEQ ID NO: 1625), NYNKKSLNVDFT (SEQ ID NO: 79 of US20160369298; herein SEQ ID NO: 1626), NYNKKSPNVDFT (SEQ ID NO: 80 of US20160369298; herein SEQ ID NO: 1627), NYSKKSEICVDFT (SEQ ID NO: 81 of US20160369298; herein SEQ ID NO: 1628), NYRKTIYVDFT (SEQ ID NO: 82 of US20160369298; herein SEQ ID NO: 1629), NYKEKKDVHFT (SEQ ID NO: 83 of US20160369298; herein SEQ ID NO: 1630), NYGHRAIVQFT (SEQ ID NO: 84 of US20160369298; herein SEQ ID NO: 1631), NYANEIQFVVCT (SEQ ID NO: 85 of US20160369298; herein SEQ ID NO: 1632), NYDDDITIGVULT (SEQ ID NO: 86 of US20160369298; herein SEQ ID NO: 1633), NYDDPTGVLLT (SEQ ID NO: 87 of US20160369298; herein SEQ ID NO: 1634), NFEQQNSVEWI (SEQ ID NO: 88 of US20160369298; herein SEQ ID NO: 1635), SQSGASN (SEQ ID NO: 89 and SEQ ID NO: 241 of US20160369298; herein SEQ ID NO: 1636), NNGSQA (SEQ ID NO: 90 of US20160369298; herein SEQ ID NO: 1637), YYLSRTNTPSGYTWSRLQFSQAGA (SEQ ID NO: 91 of US20160369298; herein SEQ ID NO: 1638), SKTSADNNNSEYSWTG (SEQ ID NO: 92 of US20160369298; herein SEQ ID NO: 1639), HKDDEEKF (SEQ ID NO: 93, 209, 214, 219, 224, 234, 239, and 244 of US20160369298; herein SEQ ID NO: 1640), KQGSEKTNVDIEEV (SEQ ID NO: 94 of US20160369298; herein SEQ ID NO: 1641), QRGNNQAATADVNT (SEQ ID NO: 95 of US20160369298; herein SEQ ID NO: 1642), NYNKKSVNVDFT (SEQ ID NO: 96 of US20160369298; herein SEQ ID NO: 1643), SQSGASNYNTPSGTTTQSRLQFSTSADNNNSEYSWTGATKYH (SEQ ID NO: 0.06 of US20160369298; herein SEQ ID NO: 1644), SASGASNFNSEGGSLTQSSLGFSTDGENNNSDFSWTGATKYH (SEQ ID NO: 107 of US20160369298; herein SEQ ID NO: 1645), SQSGASNYNTPSGTTTQSRLQFSTDGENNNSDFSWTGATKYH (SEQ ID NO: 108 of US20160369298; herein SEQ ID NO: 1646), SASGASNYNTPSGTTTQSRLQFSTSADNNNSEFSWPGATFVE (SEQ ID NO: 109 of US20160369298; herein SEQ ID NO: 1647), SQSGASNFNSEGGSLTQSSLGFSTDGENNNSDFSWTGATKYIT (SEQ TD NO: 110 of US20160369298; herein SEQ ID NO: 1648), SASGASNYNTPSGSLTQSSLGFSTDGENNNSDFSWTGATKYH (SEQ ID NO: 111 of US20160369298; herein SEQ ID NO: 1649), SQSGASNYNTPSGTTTQSRLQFSTSADNNNSDFSWTGATKYH (SEQ ID NO: 112 of US20160369298; herein SEQ ID NO: 1650), SGAGASNYNSEGGSLTQSSLGFSTDGENNNSDFSWTGATKYH (SEQ ID NO: 113 of US20160369298; herein SEQ ID NO: 1651), SGAGASN (SEQ ID NO: 176 of US20160369298; herein SEQ ID NO: 1652), NSEGGSLTQSSLGFS (SEQ ID NO: 177, 185, 193 and 202 of US20160369298; herein SEQ ID NO: 1653), TDGENNNSDFS (SEQ ID NO: 178 of US20160369298; herein SEQ ID NO: 1654), SEFSWPGATT (SEQ ID NO: 179 of US20160369298; herein SEQ ID NO: 1655), TSADNNNSDFSWT (SEQ ID NO: 180 of US20160369298, herein SEQ ID NO: 1656), SQSGASNY (SEQ ID NO: 181, 187, and 198 of US20160369298; herein SEQ ID NO: 1657), NTPSGTTTQSRLQFS (SEQ ID NO: 182, 188, 191, and 199 of US20160369298; herein SEQ ID NO: 1658), TSADNNNSEYSWTGATKYH (SEQ ID NO: 183 of US20160369298; herein SEQ ID NO: 1659), SASGASNF (SEQ ID NO: 184 of US20160369298; herein SEQ ID NO: 1660), TDGENNNSDFSWEGATKYH (SEQ ID NO: 186, 189, 194, 197, and 203 of US20160369298; herein SEQ ID NO: 1661), SASGASNY (SEQ ID NO: 190 and SEQ ID NO: 195 of US20160369298; herein SEQ ID NO: 1662), TSADNNNSEFSWPGATTYH (SEQ ID NO: 192 of 0520160369298, herein SEQ ID NO: 1663), NTPSGSLTQSSLGFS (SEQ ID NO: 196 of US20160369298; herein SEQ ID NO: 1664), TSADNNNSDFSWIGATICYFI (SEQ ID NO: 200 of US20160369298; herein SEQ ID NO: 1665), SGAGASNF (SEQ ID NO: 201 of US20160369298; herein SEQ ID NO: 1666), CTCCAGVVSVVSMRSRVCVNSGCAGCTDHCVNTSRNSGTCVMSACACAA (SEQ ID NO: 204 of US20160369298; herein SEQ ID NO: 1667), CTCCAGAGAGGCAACAGACAAGCAGCTACCGCAGATGTCAACACACAA (SEQ ID NO: 205 of US20160369298, herein SEQ ID NO: 1668), SAAGASN (SEQ ID NO: 206 of US20160369298; herein SEQ ID NO: 1669), YFLSRTNTESGSTTQSTLRFSQAG (SEQ ID NO: 207 of US20160369298; herein SEQ ID NO: 1670), SKTSADNNNSDFS (SEQ ID NO: 208, 228, and 253 of US20160369298; herein SEQ ID NO: 1671), KQGSEKTDVDIDKV (SEQ ID NO: 210 of US20160369298; herein SEQ ID NO: 1672), STAGASN (SEQ ID NO: 211 of US20160369298; herein SEQ ID NO: 1673), V_{L}SRTNTTSGIETQSTLRFSQAG (SEQ ID NO: 212 and SEQ ID NO: 247 of US20160369298, herein SEQ ID NO: 1674), SKIDGENNNSDFS (SEQ ID NO: 213 and SEQ ID NO: 248 of US20160369298; herein SEQ ID NO: 1675), KQGAAADDVEIDGV (SEQ ID NO: 215 and SEQ ID NO: 250 of US20160369298, herein SEQ ID NO: 1676), SEAGASN (SEQ ID NO: 216 of US20160369298; herein SEQ ID NO: 1677), YYILSRTNTPSGTTTQSRISQFSQAG (SEQ ID NO: 217, 232 and 242 of US20160369298; herein SEQ ID NO: 1678), SKTSADNNNSEYS (SEQ ID NO: 218, 233, 238, and 243 of US20160369298; herein SEQ ID NO: 1679), KQGSEKTNVDIEKV (SEQ ID NO: 220, 225 and 245 of US20160369298; herein SEQ TD NO: 1680), YFLSRTNDASGSDIKSTLLFSQAG (SEQ ID NO: 222 of US20160369298; herein SEQ ID NO: 1681), STTPSENNNSEYS (SEQ ID NO: 223 of US20160369298; herein SEQ ID NO: 1682), SAAGATN (SEQ ID NO: 226 and SEQ ID NO: 251 of US20160369298; herein SEQ ID NO: 1683), YFLSRTNGEAGSATLSELRFSQAG (SEQ ID NO: 227 of US20160369298; herein SEQ ID NO: 1684), HGDDADRF (SEQ ID NO: 229 and SEQ ID NO: 254 of US20160369298; herein SEQ ID NO: 1685), KQGAFKSDVEVDRV (SEQ ID NO: 230 and SEQ ID NO: 255 of US20160369298; herein SEQ ID NO: 1686), KQDSGGDNIDIDQV (SEQ ID NO: 235 of US20160369298; herein SEQ ID NO: 1687), SDAGASN (SEQ ID NO: 236 of US20160369298; herein SEQ ID NO: 1688), YFLSRTNTEGGHDTQSTLRFSQAG(SEQ ID NO: 237 of US20160369298, herein SEQ ID NO: 1689), KEDGGGSDVAIDEV (SEQ ID NO: 240 of US20160369298; herein SEQ ID NO: 1690), SDAGASN (SEQ ID NO: 246 of 0S20160369298; herein SEQ ID NO: 1691), and YFLSRTNGEAGSATLSELRFSQPG (SEQ ID NO: 252 of US20160369298; herein SEQ ID NO: 1692), Non-limiting examples of nucleotide sequences that may encode the amino acid mutated sites include the following, AGCVVMDCAGGARSCASCAAC (SEQ ID NO: 97 of US20160369298; herein SEQ ID NO: 1693), AACRACRRSMRSMAGGCA (SEQ ID NO: 98 of US20160369298; herein SEQ ID NO: 1694), CACRRGGACRRCRMSRRSARSTTT (SEQ ID NO: 99 of US20160369298; herein SEQ ID NO: 1695), TATTTCTTGAGCAGAACAAACRVCVVSRSCGGAMNCVHSACGMHSTCAVVSCTTVDS TTTTCTCAGSBCRGSGCG (SEQ ID NO: 100 of US20160369298; herein SEQ ID NO: 1696), TCAAMANLMAVNSRVCSRSAACAACAACAGTRASTTCTCGTGGMMAGGA (SEQ ID NO: 101 of US20160369298; herein SEQ ID NO: 1697), AAGSAARRERSCRVSRVARVCRATRYCGMSNFICRVMVRSGTC (SEQ ID NO: 102 of US20160369298, herein SEQ ID NO: 1698), CAGVVSVVSMRSRVCVNSGCAGCTDHCVVSRNSGTCVMSACA (SEQ ID NO: 103 of US20160369298; herein SEQ ID NO: 1699), AACTWCRVSVASMVSVHSDDTGTGSWSTKSACT (SEQ ID NO: 104 of US20160369298; herein SEQ ID NO: 1700), TTGTTGAACATCACCACGTGACGCACGTTC (SEQ ID NO: 256 of US20160369298; herein SEQ ID NO: 1701), TCCCCGTGGTTCTACTACATAATGTGGCCG (SEQ ID NO: 257 of US20160369298; herein SEQ TD NO: 1702). TTCCACACTCCGTTTTGGATAATGTTGAAC (SEQ ID NO: 258 of US20160369298; herein SEQ ID NO: 1703), AGGGACATCCCCAGCTCCATGCTGTGGTCG (SEQ ID NO: 259 of US20160369298; herein SEQ ID NO: 1704), AGGGACAACCCCTCCGACTCGCCCTAATCC (SEQ ID NO: 260 of US20160369298; herein SEQ ID NO: 1705), TCCTAGTAGAAGACACCCTCTCACTGCCCG (SEQ ID NO: 261 of US20160369298; herein SEQ ID NO: 1706), AGTACCATGTACACCCACTCTCCCAGTGCC (SEQ ID NO: 262 of US20160369298; herein SEQ ID NO: 1707), ATATGGACGTTCATGCTGATCACCATACCG (SEQ ID NO: 263 of US20160369298; herein SEQ ID NO: 1708), AGCAGGAGCTCCTTGGCCTCAGCGTGCGAG (SEQ ID NO: 264 of US20160369298; herein SEQ ID NO: 1709), ACAAGCAGCTTCACTATGACAACCACTGAC (SEQ ID NO: 265 of US20160369298; herein SEQ ID NO: 1710), CAGCCTAGGAACTGGCTICCTGGACCCTGITACCGCCAGCAGAGAGTCTCAAMAMM AVNSRVCSRSAACAACAACAGTRASTTCTCCTGGMMAGGAGCTACCAAGTACCACC TCAATGGCAGAGACTCTCTGGTGAATCCCGGACCAGCTATGGCAAGCCACRRGGAC RRCRIVISRRSARSTTITTTCCTCAGAGCGGGGTTCTCATCTITGGGAAGSAARRCRSCR VSRVARVCRATRYCGMSNHCRVMVRSGTCATGATTACAGACGAAGAGGAQATCTGG AC (SEQ ID NO: 266 of 11520160369298; herein SEQ ID NO: 1711), TGGGACAATGGCGGTCGTCTCTCAGAGTTKTKKT (SEQ ID NO: 267 of US20160369298; herein SEQ ID NO: 1712), AGAGGACCKKTCCTCGATGGTTCATGGTGGAGTTA (SEQ ID NO: 268 of US20160369298; herein SEQ ID NO: 1713), CCACTTAGGGCCTGGTCGATACCGTTCGGTG (SEQ ID NO: 269 of US20160369298; herein SEQ ID NO: 1714), and TCTCGCCCCAAGAGTAGAAACCCTTCSTTYYG (SEQ ID NO: 270 of US20160369298; herein SEQ ID NO: 1715).

In some embodiments, the AAV serotype may comprise an ocular cell targeting peptide as described in International Patent Publication WO2016134375, the contents of which are herein incorporated by reference in their entirety, such as, but not limited to SEQ ID NO: 9, and SEQ ID N0:10 of WO2016134375. Further, any of the ocular cell targeting peptides or amino acids described in WO2016134375, may be inserted into any parent AAV serotype, such as, but not limited to, AAV2 (SEQ ID NO:8 of WO2016134375; herein SEQ ID NO: 1716), or AAV9 (SEQ ID NO: 11 of WO2016134375; herein SEQ ID NO: 1717). In some embodiments, modifications, such as insertions are made in AAV2 proteins at P34-A35, T138-A1.39, A139-P140, G453-1454, N587-R588, and/or R588-Q589. In certain embodiments, insertions are made at D384, G385, 1560, T561, N562, E563, E564, E565, N704, and/or Y705 of AAV9. The ocular cell targeting peptide may be, but is not limited to, any of the following amino acid sequences, GSTPPPM (SEQ ID NO: 1 of WO2016134375; herein SEQ ID NO: 1718), or GETRAPL (SEQ ID NO: 4 of WO20161.34375; herein SEQ ID NO: 1719).

In some embodiments, the AAV serotype may be modified as described in the United States Publication US 20170145405 the contents of which are herein incorporated by reference in their entirety. AAV serotypes may include, modified AAV2 (e.g., modifications at Y444F, Y500F, Y730F and/or 5662V), modified AAV3 (e.g., modifications at Y705F, Y731F and/or T492V), and modified AAV6 (e.g., modifications at 5663V and/or T492V).

In some embodiments, the AAV serotype may be modified as described in the International Publication WO2017083722 the contents of which are herein incorporated by reference in their entirety. AAV serotypes may include, AAV1 (Y705+731F+T492V); AAV2 (Y444+500+730F+T491V), AAV3 (Y705+731F), AAV5, AAV 5(Y436+693+719F), AAV6 (VP3 variant Y705F/Y731F/T492V), AAV8 (Y733F), AAV9, AAV9 (VP3 variant Y731F), and AAV10 (Y733F).

In some embodiments, the AAV serotype may comprise, as described in International Patent Publication WO2017015102, the contents of which are herein incorporated by reference in their entirety, an engineered epitope comprising the amino acids SPAKFA (SEQ ID NO: 24 of WO2017015102; herein SEQ ID NO: 1720) or NKDKLN (SEQ ID NO:2 of WO2017015102; herein SEQ ID NO: 1721). The epitope may be inserted in the region of amino acids 665 to 670 based on the numbering of the VP1 capsid of AAV8 (SEQ ID NO: 3 of WO2017015102) and/or residues 664 to 668 of AAV 313 (SEQ ID NO: 3).

In some embodiments, the AAV serotype may be, or may have a sequence as described in international Patent Publication WO2017058892, the contents of which are herein incorporated by reference in their entirety, such as, but not limited to, AAV variants with capsid proteins that may comprise a substitution at one or more (e.g., 2, 3, 4, 5, 6, or 7) of amino acid residues 262-268, 370-379, 451-459, 472-473, 493-500, 528-534, 547-552, 588-597, 709-710, 716-722 of AAV1, in any combination, or the equivalent amino acid residues in AAV2, AAV3, AAV4, AAV5, AAV6, AAV7, AAV8, AAV9, AAV10, AAV11, AAV12, AAVrh8, AAVrh10, AAVrh32.33, bovine AAV or avian AAV. The amino acid substitution may be, but is not limited to, any of the amino acid sequences described in WO2017058892. In some embodiments, the AAV may comprise an amino acid substitution at residues 256L, 258K, 259Q, 2615, 263A, 264S, 265T, 266G, 2721-I, 385S, 386Q, 5472R, V473D, N500E 547S, 709A, 710N, 716D, 717N, 718N, 720L, A456T, Q457T, N458Q, K4595, T4925, K493A, 55868, 5587G, 5588N, T589R and/or 722T of AAV1 (SEQ ID NO: 1 of WO2017058892) in any combination, 244N, 246Q, 248R, 249E, 2501, 251K, 252S, 253G, 254S, 255V, 2561, 263Y, 377E, 378N, 453L, 456R, 532Q, 533P, 535N, 536P, 537G, 538T, 539T, 540A, 541T, 542Y, 543L, 546N, 653V, 654P, 656S, 697Q, 698F, 704D, 705S, 706T, 707G, 708E, 709Y and; or 710R of AAV5 (SEQ ID NO:5 of WO2017058892) in any combination, 248R, 316V, 317Q, 318D, 319S, 443N, 530N, 5315, 532Q 533P, 534A, 535N, 540A, 541 T, 542Y, 5431, 545G, 546N, 697Q, 704D, 706T, 708E, 709Y and/or 710R of AAV5 (SEQ ID NO: 5 of WO2017058892) in any combination, 264S, 266G, 269N, 272H, 457Q, 588S and/or 5891 of AAV6 (SEQ ID NO:6 WO2017058892) in any combination, 457T, 459N, 496G, 499N, 500N, 589Q, 590N and/or 592A of AAV8 (SEQ ID NO: 8 WO2017058892) in any combination, 4511, 452N, 453G, 454S, 455G, 456Q, 457N and/or 458Q of AAV9 (SEQ ID NO: 9 WO2017058892) in any combination.

In some embodiments, the AAV may include a sequence of amino acids at positions 155, 156 and 157 of VP1 or at positions 17, 18, 19 and 20 of VP2, as described in International Publication No. WO 2017066764, the contents of which are herein incorporated by reference in their entirety. The sequences of amino acid may be, but not limited to, N-S-S, S-X-S, S-S-Y, N-X-S, N-S-Y, S-X-Y and N-X-Y, where N, X and Y are, but not limited to, independently non-serine, or non-threonine amino acids, wherein the AAV may be, but not limited to AAV1, AAV2, AAV3, AAV4, AAV5. AAV6, AAV7, AAV8, AAV9, AAV1.0, AAT 11 and AAV12. In some embodiments, the AAV may include a deletion of at least one amino acid at positions 156, 157 or 158 of VP1 or at positions 19, 20 or 21 of VP2, wherein the AAV may be, but not limited to AAV1, AAV2, AAV3, AAV4, AAV5, AAV6, AAV7, AAV8, AAV9, AAV10, AAV11 and AAV12.

In some embodiments, the AAV may be a serotype generated by Cre-recombination-based AAV targeted evolution (CREATE) as described by Deverman et al., (Nature Biotechnology 34(2):204209 (2016)), the contents of which are herein incorporated by reference in their entirety. In some embodiments, AAV serotypes generated in this manner have improved CNS transduction and/or neuronal and astrocytic tropism, as compared to other AAV serotypes. As non-limiting examples, the AAV serotype may include a peptide such as, but not limited to, PHP.B, PHP.B2, PHP.B3, PHP.A, G2A.12, G2A15, G2A3, 02134, and G2135, In some embodiments, these AAV serotypes may be AAV9 (SEQ ID NO: 9 or 136) derivatives with a 7-amino acid insert, between amino acids 588-589. Non-limiting examples of these 7-amino acid inserts include TLAVPFK (PHP.B; SEQ ID NO: 1260), SVSKPFL (PHP.B2; SEQ ID NO: 1268), FTLTTPK (PHP.B3; SEQ ID NO: 1269), YTLSQGW (PHP.A; SEQ ID NO: 1275), QAVRTSL (PHP.S; SEQ ID NO: 1319), LAKERLS G2A3; SEQ ID NO: 1320), MNSTKNV (G2B4; SEQ ID NO: 1321), and/or VSGGHHS (G2B5; SEQ ID NO: 1322).

In some embodiments, the AAV serotype may be as described in Jackson et al (Frontiers in Molecular Neuroscience 9:154 (2016)), the contents of which are herein incorporated by reference in their entirety. In some embodiments, the AAV serotype is PHP.B or AAV9. In some embodiments, the AAV serotype is paired with a synapsin promoter to enhance neuronal transduction, as compared to when more ubiquitous promoters are used (i.e., CBA or CMV).

In some embodiments, the AAV serotype is a serotype comprising the AAVPHP.N (PHP.N) peptide, or a variant thereof.

In some embodiments the AAV serotype is a serotype comprising the AAVPHP.B (PHP.B) peptide, or a variant thereof.

In some embodiments, the AAV serotype is a serotype comprising the AAVPHP.A (PHP.A) peptide, or a variant thereof.

In some embodiments, the AAV serotype is a serotype comprising the PHP.S peptide, or a variant thereof.

In some embodiments, the AAV serotype is a serotype comprising the PHP.B2 peptide, or a variant thereof.

In some embodiments, the AAV serotype is a serotype comprising the PHP.B3 peptide, or a variant thereof.

In some embodiments, the AAV serotype is a serotype comprising the G2B4 peptide, or a variant thereof.

In some embodiments, the AAV serotype is a serotype comprising the G2135 peptide, or a variant thereof.

In some embodiments the AAV serotype is VOY1.01, or a variant thereof.

In some embodiments, the AAV serotype is VOY201, or a variant thereof.

### Viral Genome Component: Inverted Terminal Repeats (ITRs)

The AAV particles of the present disclosure comprise a viral genome with at least one ITR region and a payload region. In some embodiments, the viral genome has two ITRs. These two Wits flank the payload region at the 5′ and 3′ ends. The ITRs function as origins of replication comprising recognition sites for replication. ITRs comprise sequence regions which can be complementary and symmetrically arranged. ITRs incorporated into viral genomes of the disclosure may be comprised of naturally occurring polynucleotide sequences or recombinantly derived polynucleotide sequences.

The ITRs may be derived from the same serotype as the capsid, selected from any of the serotypes listed in Table 1, or a derivative thereof. The ITR may be of a different serotype than the capsid. In some embodiments, the AAV particle has more than one ITR. In a non-limiting example, the AAV particle has a viral genome comprising two ITRs. In some embodiments, the ITRs are of the same serotype as one another. In another embodiment, the ITRs are of different serotypes. Non-limiting examples include zero, one or both of the ITRs having the same serotype as the capsid. In some embodiments both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-415 nucleotides in length, 116-120 nucleotides in length, 121-125 nucleotides in length, 126-130 nucleotides in length, 131-435 nucleotides in length, 136140 nucleotides in length, 141-445 nucleotides in length or 146-150 nucleotides in length. In some embodiments, the ITRs are 140-142 nucleotides in length. Non-limiting examples of ITR length are 102, 130, 140, 141, 142, 145 nucleotides in length, and those having at least 95% identity thereto.

In some embodiments, each ITR may be 141 nucleotides in length.

In some embodiments, each ITR may be 130 nucleotides in length.

In some embodiments, the AAV particles comprise two ITRs and one ITR is 141 nucleotides in length and the other ITR is 130 nucleotides in length.

### Viral Genome Component: Promoters

In some embodiments, the payload region of the viral genome comprises at least one element to enhance the transgene target specificity and expression (See e.g., Powell et al. Viral Expression Cassette Elements to Enhance Transgene Target Specificity and Expression in Gene Therapy, 2015; the contents of which are herein incorporated by reference in its entirety). Non-limiting examples of elements to enhance the transgene target specificity and expression include promoters, endogenous miRNAs, post-transcriptional regulatory elements (PREs), polyadenylation (PolyA) signal sequences and upstream enhancers (USEs), CMV enhancers and introns.

A person skilled in the art may recognize that expression of the polypeptides of the disclosure in a target cell may require a specific promoter, including but not limited to, a promoter that is species specific, inducible, tissue-specific, or cell cycle-specific (Parr et al., Nat. Med 3:1145-9 (1997); the contents of which are herein incorporated by reference in their entirety).

In some embodiments, the promoter is deemed to be efficient when it drives expression of the polypeptide(s) encoded in the payload region of the viral genome of the AAV particle.

In some embodiments, the promoter is a promoter deemed to be efficient when it drives expression in the cell being targeted.

In some embodiments, the promoter drives expression of the polypeptides of the disclosure (e.g., a functional antibody) for a period of time in targeted tissues. Expression driven by a promoter may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years, or 5-10 years.

In some embodiments, the promoter drives expression of the polypeptides of the disclosure (e.g., a functional antibody) for at least 1 month, 2 months, 3 months, 4 months, 5 months, 6 months, 7 months, 8 months, 9 months, 10 months, 11 months, 1 year, 2 years, 3 years 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years.

Promoters may be naturally occurring or non-naturally occurring. Non-limiting examples of promoters include viral promoters, plant promoters and mammalian promoters. In some embodiments, the promoters may be human promoters. In some embodiments, the promoter may be truncated.

Promoters which drive or promote expression in most tissues include, but are not limited to, human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C(UBC). Tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes.

Non-limiting examples of muscle-specific promoters include mammalian muscle creatine kinase (NICK) promoter, mammalian desmin (DES) promoter, mammalian troponin I (TNNI2) promoter, and mammalian skeletal alpha-actin (ASKA) promoter (see, e.g. U.S. Patent Publication US20110212529 the contents of which are herein incorporated by reference in their entirety)

Non-limiting examples of tissue-specific expression elements for neurons include neuron-specific enolase (NSE), platelet-derived growth factor (PDGF), platelet-derived growth factor B-chain (PDGF-β), synapsin (Syn), methyl-CpG binding protein 2 (MeCP2), Ca²⁺/calmodulin-dependent protein kinase II (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter.

In some embodiments, the promoter may be less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or more than 800 nucleotides. The promoter may have a length between 200-300, 200-400, 200-500, 200-600, 200-700, 200-800, 300-400, 300-500, 300-600, 300-700, 300-800, 400-500, 400-600, 400-700, 400-800, 500-600-500-700, 500-800, 600-700, 600-800, or 700-800.

In some embodiments, the promoter may be a combination of two or more components of the same or different starting or parental promoters such as, but not limited to, CMV and CBA. Each component may have a length of 200, 210, 220, 230, 240, 250, 260, 270, 280, 290, 300, 310, 320, 330, 340, 350, 360, 370, 380, 381, 382, 383, 384, 385, 386, 387, 388, 389, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or more than 800. Each component may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800. In some embodiments, the promoter is a combination of a 382 nucleotide CMV-enhancer sequence and a 260 nucleotide CBA-promoter sequence.

In some embodiments, the viral genome comprises a ubiquitous promoter. Non-limiting examples of ubiquitous promoters include CMV, CBA (including derivatives CAG, CBh, etc.), EF-1α, PGK, UBC, GUSB (hGBp), and UCOE (promoter of HNRPA2B1-CBX3).

Yu et al. (Molecular Pain 2011, 7:63; the contents of which are herein incorporated by reference in their entirety) evaluated the expression of eGFP under the CAG, and UBC promoters in rat DRG cells and primary DRG cells using lentiviral vectors and found that UBC showed weaker expression than the other 3 promoters and only 1012% glial expression was seen for all promoters. Soderblom et al. (E. Neuro 2015; the contents of which are herein incorporated by reference in its entirety) evaluated the expression of eGFP in AAV8 with CMV and UBC promoters and AAV2 with the CMV promoter after injection in the motor cortex. Intranasal administration of a plasmid containing a UBC or EFIα promoter showed a sustained airway expression greater than the expression with the CMV promoter (See e.g., Gill et al., Gene Therapy 2001, Vol. 8, 1539-1546; the contents of which are herein incorporated by reference in their entirety). Husain et al. (Gene Therapy 2009; the contents of which are herein incorporated by reference in its entirety) evaluated an HβH construct with a hGUSB promoter, a HSV-1LAT promoter and an NSE promoter and found that the HβH construct showed weaker expression than NSE in mouse brain. Passini and Wolfe (J. Virol. 2001, 12382-12392, the contents of which are herein incorporated by reference in its entirety) evaluated the long-term effects of the HβH vector following an intraventricular injection in neonatal mice and found that there was sustained expression for at least 1 year. Low expression in all brain regions was found by Xu et al. (Gene Therapy 2001, 8, 1323-1332; the contents of which are herein incorporated by reference in their entirety) when NFL and NFH promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650nucleotide promoter and NFH is a 920 nucleotide promoter which are both absent in the liver but NFH is abundant in the sensory proprioceptive neurons, brain and spinal cord and MIT is present in the heart. Scn8a is a 470 nucleotide promoter which expresses throughout the DRG, spinal cord and brain with particularly high expression seen in the hippocampal neurons and cerebellar Purkinje cells, cortex, thalamus, and hypothalamus (See e.g., Drews et al. Identification of evolutionary conserved functional noncoding elements in the promoter region of the sodium channel gene SCN8A, Mamm Genome (2007) 18:723-731; and Raymond et al. Expression of Alternatively Spliced Sodium Channel α-subunit genes, Journal of Biological Chemistry (2004) 279(44) 46234-46241; the contents of each of which are herein incorporated by reference in their entireties).

Any of promoters taught by the aforementioned Yu, Soderblom, Gill, Husain, Passini, Xu, Drews, or Raymond may be used.

In some embodiments, the promoter is not cell specific.

In some embodiments, the promoter is a ubiquitin c (UBC) promoter. The UBC promoter may have a size of 300-350 nucleotides. As a non-limiting example, the UBC promoter is 332 nucleotides.

In some embodiments, the promoter is a P-glucuronidase (GUSB) promoter. The GUS:13 promoter may have a size of 350-400 nucleotides. As a non-limiting example, the GUSB promoter is 378 nucleotides.

In some embodiments, the promoter is a neurofilament light (NFL) promoter. The NFL promoter may have a size of 600700 nucleotides. As a non-limiting example, the NFL, promoter is 650 nucleotides.

In some embodiments, the promoter is a neurofilament heavy (NFH) promoter. The NFH promoter may have a size of 900-950 nucleotides. As a non-limiting example, the NFL promoter is 920 nucleotides.

In some embodiments, the promoter is a scn8a promoter. The scn8a promoter may have a size of 450-500 nucleotides. As a non-limiting example, the scn8a promoter is 470 nucleotides.

In some embodiments, the promoter is a phosphoglycerate kinase 1 (PGK) promoter.

In some embodiments, the promoter is a chicken β-actin (CBA) promoter.

In some embodiments, the promoter is a CB6 promoter.

In some embodiments, the promoter is a minimal CB promoter.

In some embodiments, the promoter is a cytomegalovirus (CMV) promoter.

In some embodiments, the promoter i s a CAG promoter.

In some embodiments, the promoter is a GFAP promoter.

In some embodiments, the promoter is a synapsin promoter.

In some embodiments, the promoter is a liver or a skeletal muscle promoter. Non-limiting examples of liver promoters include human α-1-antitrypsin (hAAT) and thyroxine binding globulin (TBG). Non-limiting examples of skeletal muscle promoters include Desmin, MCK or synthetic C5-12.

In some embodiments, the promoter is a RNA pol III promoter. As a non-limiting example, the RNA pol III promoter is U6. As a non-limiting example, the RNA pol III promoter is H1.

In some embodiments, the viral genome comprises two promoters. As a non-limiting example, the promoters are an EF1α promoter and a CMV promoter.

In some embodiments, the viral genome comprises an enhancer element, a promoter and/or a 5′UTR intron. The enhancer element, also referred to herein as an “enhancer,” may be, but is not limited to, a CMV enhancer, the promoter may be, but is not limited to, a CMV, CBA, UBC, GUSB, NSE, Synapsin, MeCP2, and GFAP promoter and the 5′UTR/intron may be, but is not limited to, SV40, and CBA-MVM. As a non-limiting example, the enhancer, promoter and/or intron used in combination may be: (1) CMV enhancer, CMV promoter, SA/40 5′UTR intron; (2) CMV enhancer, CBA promoter, SV 40 5′ UTR intron; (3) CMV enhancer, CBA promoter, CBA-MVM 5′UTR intron; (4) UBC promoter; (5) GUSB promoter; (6) NSE promoter; (7) Synapsin promoter; (8) MeCP2 promoter; and (9) GFAP promoter.

In some embodiments, the viral genome comprises an engineered promoter.

In another embodiment, the viral genome comprises a promoter from a naturally expressed protein.

### Viral Genome Component: Untranslated Regions (UTRs)

By definition, wild type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs may be engineered into UTRs to enhance the stability and protein production. As a non-limiting example, a 5′ UTR from mRNA normally expressed in the liver (e.g., albumin, serum amyloid A, Apolipoprotein A/B/E, transferrin, alpha fetoprotein, erythropoietin, or Factor VIII) may be used in the viral genomes of the AAV particles of the disclosure to enhance expression in hepatic cell lines or liver.

While not wishing to be bound by theory, wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCR(A/G)CCAUGG, where R is a purine (adenine or guanine) three bases upstream of the start codon (ATG), which is followed by another ‘G’.

In some embodiments, the 5′UTR in the viral genome includes a Kozak sequence.

In some embodiments, the 5′UTR in the viral genome does not include a Kozak sequence.

While not wishing to be bound by theory, wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety): Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping UUAUUUA(U/A)(U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In some embodiments, the 3′ UTR of the viral genome may include an oligo(dT) sequence for templated addition of a poly-A tail.

In some embodiments, the viral genome may include at least one miRNA seed, binding site or full sequence microRNAs (or miRNA or miR) are 19-25 nucleotide noncoding RNAs that bind to the sites of nucleic acid targets and down-regulate gene expression either by reducing nucleic acid molecule stability or by inhibiting translation. A microRNA sequence comprises a “seed” region, i.e., a sequence in the region of positions 2-8 of the mature microRNA, which sequence has perfect Watson-Crick complementarity to the miRNA target sequence of the nucleic acid.

In some embodiments, the viral genome may be engineered to include, alter or remove at least one miRNA binding site, sequence, or seed region.

Any UTR from any gene known in the art may be incorporated into the viral genome of the AAV particle. These UTRs, or portions thereof, may be placed in the same orientation as in the gene from which they were selected or they may be altered in orientation or location. In some embodiments, the UTR used in the viral genome of the AAV particle may be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In some embodiments, the viral genome of the AAV particle comprises at least one artificial UTRs which is not a variant of a wild type UTR.

In some embodiments, the viral genome of the AAV particle comprises UTRs which have been selected from a family of transcripts whose proteins share a common function, structure, feature or property.

### Viral Genome Component: Polyadenylation Sequence

In some embodiments, the viral genome of the AAV particles of the present disclosure comprise at least one polyadenylation sequence. The viral genome of the AAV particle may comprise a polyadenylation sequence between the 3′ end of the payload coding sequence and the 5′ end of the 3′ITR.

In some embodiments, the polyadenylation sequence or “polyA sequence” may range from absent to about 500 nucleotides in length. The polyadenylation sequence may be, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, and 600 nucleotides in length.

In some embodiments, the polyadenylation sequence is 50-100 nucleotides in length.

In some embodiments, the polyadenylation sequence is 50-150 nucleotides in length.

In some embodiments, the polyadenylation sequence is 50-160 nucleotides in length.

In some embodiments, the polyadenylation sequence is 50-200 nucleotides in length.

In some embodiments, the polyadenylation sequence is 60-100 nucleotides in length.

In some embodiments, the polyadenylation sequence is 60-150 nucleotides in length.

In some embodiments, the polyadenylation sequence is 60-160 nucleotides in length.

In some embodiments, the polyadenylation sequence is 60-200 nucleotides in length.

In some embodiments, the polyadenylation sequence is 70-100 nucleotides in length.

In some embodiments, the polyadenylation sequence is 70-150 nucleotides in length.

In some embodiments, the polyadenylation sequence is 70-160 nucleotides in length.

In some embodiments, the polyadenylation sequence is 70-200 nucleotides in length.

In some embodiments, the polyadenylation sequence is 80-100 nucleotides in length.

In some embodiments, the polyadenylation sequence is 80-150 nucleotides in length.

In some embodiments, the polyadenylation sequence is 80-160 nucleotides in length.

In some embodiments, the polyadenylation sequence is 80-200 nucleotides in length.

In some embodiments, the polyadenylation sequence is 90-100 nucleotides in length.

In some embodiments, the polyadenylation sequence is 90-150 nucleotides in length.

In some embodiments, the polyadenylation sequence is 90-160 nucleotides in length.

In some embodiments, the polyadenylation sequence is 90-200 nucleotides in length.

In some embodiments, the polyadenylation sequence is 127 nucleotides in length.

In some embodiments, the polyadenylation sequence is 477 nucleotides in length.

In some embodiments, the polyadenylation sequence is 552 nucleotides in length.

### Viral Genome Component: Linkers

Viral genomes of the disclosure may be engineered with one or more spacer or linker regions to separate coding or non-coding regions.

In some embodiments, the payload region of the AAV particle may optionally encode one or more linker sequences. In some cases, the linker may be a peptide linker that may be used to connect the polypeptides encoded by the payload region (i.e., light and heavy antibody chains during expression). Some peptide linkers may be cleaved after expression to separate heavy and light chain domains, allowing assembly of mature antibodies or antibody fragments. Linker cleavage may be enzymatic. In some cases, linkers comprise an enzymatic cleavage site to facilitate intracellular or extracellular cleavage. Some payload regions encode linkers that interrupt polypeptide synthesis during translation of the linker sequence from an mRNA transcript. Such linkers may facilitate the translation of separate protein domains (e.g., heavy and light chain antibody domains) from a single transcript. In some cases, two or more linkers are encoded by a payload region of the viral genome. Non-limiting examples of linkers that may be encoded by the payload region of an AAV particle viral genome are given in Table 2.

TABLE 2
Linkers

| | | SEQ ID |
|---|---|---|
| Lin- | | NO or |
| ker | | SE- |
| No. | Description | QUENCE |
| L1 | Internal ribosome entry site (IRES) | 1724 |
| L2 | Foot and mouth disease virus 2A (F2A) | 1725 |
| L3 | Porcine teschovirus-1 virus 2A (P2A) | 1726 |
| L4 | Furin cleavage site (F) | 1727 |
| L5 | 5xG4S (“5xG4S” amino acid sequence | 1728 |
| | disclosed as SEQ ID NO: 32689) | |
| L6 | Furin-foot and mouth disease virus 2A (F.F2A) | 1729 |
| L7 | Furin-porcine teschovirus-1 virus 2A (F.P2A) | 1730 |
| L8 | Furin cleavage site variant (F1) | 1731 |
| L9 | Furin Thoseaasigna virus 2A (Furin2A) | 1732 |
| L10 | G4S (“G4S” amino acid sequence | 1733 |
| | disclosed as SEQ ID NO: 2443) | |
| L11 | G4S3 (“G4S3” amino acid sequence | 1734 |
| | disclosed as SEQ ID NO: 2449) | |
| L12 | hIgG2 hinge | 1735 |
| L13 | hIgG3 hinge | 1736 |
| L14 | hIgG3-2 hinge | 1737 |
| L15 | hIgG3-3 hinge | 1738 |
| L16 | IRES-2 | 1739 |
| L17 | msiGG-1 hinge | 1740 |
| L18 | msiGG1 hinge | 1741 |
| L19 | Thoseaasigna virus 2A (T2A) | 1742 |
| L20 | 1,4-alpha-glucan-branching enzyme | CHP |
| L21 | 1,4-alpha-glucan-branching enzyme | 1743 |
| L22 | 1,4-beta-N-acetylmuramidase | FKK |
| L23 | 1,4-beta-N-acetylmuramidase | 1744 |
| L24 | 1,4-beta-N-acetylmuramidase | 1745 |
| L25 | 1,4-beta-N-acetylmuramidase | 1746 |
| L26 | 1.4-beta-N-acetylmuramidase | 1747 |
| L27 | 1,4-beta-N-acetylmuramidase | 1748 |
| L28 | 1,4-beta-N-acetylmuramidase | 1749 |
| L29 | 1,4-beta-N-acetylmuramidase | 1750 |
| L30 | 1,4-beta-N-acetylmuramidase | 1751 |
| L31 | 1,4-beta-N-acetylmuramidase | 1752 |
| L32 | 1,4-beta-N-acetylmuramidase | 1753 |
| L33 | 150aa long hypothetical transcriptional regulator | 1754 |
| L34 | 150aa long hypothetical transcriptional regulator | 1755 |
| L35 | 1-deoxy-D-xylulose 5-phosphate reductoisomerase | 1756 |
| L36 | 1-deoxy-D-xylulose 5-phosphate reductoisomerase | 1757 |
| L37 | 1-deoxy-D-xylulose 5-phosphate reductoisomerase | 1758 |
| L38 | 1-deoxy-D-xylulose 5-phosphate reductoisomerase | 1759 |
| L39 | 235aa long hypothetical biotin- | 1760 |
| | [acetyl-CoA-carboxylase] ligase | |
| L40 | 235aa long hypothetical biotin- | 1761 |
| | [acetyl-CoA-carboxylase] ligase | |
| L41 | 235aa long hypothetical biotin- | 1762 |
| | [acetyl-CoA-carboxylase] ligase | |
| L42 | 2-dehydropantoate 2-reductase | 1763 |
| L43 | 2-dehydropantoate 2-reductase | 1764 |
| L44 | 2-dehydropantoate 2-reductase | 1765 |
| L45 | 2-dehydropantoate 2-reductase | 1766 |
| L46 | 2-dehydropantoate 2-reductase | 1767 |
| L47 | 2-dehydropantoate 2-reductase | 1768 |
| L48 | 2-dehydropantoate 2-reductase, putative | 1769 |
| L49 | 2-dehydropantoate 2-reductase, putative | 1770 |
| L50 | 4-alpha-glucanotransferase | 1771 |
| L51 | 4-alpha-glucanotransferase | 1772 |
| L52 | 4-alpha-glucanotransferase | 1773 |
| L53 | 4-diphosphocytidyl-2C-methyl-D-erythritol kinase | HAA |
| L54 | 4-diphosphocytidyl-2C-methyl-D-erythritol kinase | 1774 |
| L55 | 4-diphosphocytidyl-2C-methyl-D-erythritol kinase | 1775 |
| L56 | 4-diphosphocytidyl-2C-methyl-D-erythritol kinase | 1776 |
| L57 | 4-diphosphocytidyl-2C-methyl-D-erythritol kinase | 1777 |
| L58 | 4-hydroxyphenylpyruvate dioxygenase | 1778 |
| L59 | 5-13 amino acids from the N termini | 1779 |
| | of human Ck and CH1 domains linker | |
| L60 | 5-13 amino acids from the N termini | ERK |
| | of human Ck and CH1 domains linker | |
| L61 | 5-13 amino acids from the N termini | 1780 |
| | of human Ck and CH1 domains linker | |
| L62 | 5-13 amino acids from the N termini | 1781 |
| | of human Ck and CH1 domains linker | |
| L63 | 5-13 amino acids from the N termini | 1782 |
| | of human Ck and CH1 domains linker | |
| L64 | 5-13 amino acids from the N termini | 1783 |
| | of human Ck and CH1 domains linker | |
| L65 | 5′-exonuclease | 1784 |
| L66 | 5-methyltetrahydropteroyltriglutamate— | ARL |
| | homocysteinemethyltransferase | |
| L67 | 5-methyltetrahydropteroyltriglutamate | 1785 |
| | homocysteinemethyltransferase | |
| L68 | 5-methyltetrahydropteroyltriglutamate | 1786 |
| | homocysteinemethyltransferase | |
| L69 | 5-methyltetrahydropteroyltriglutamate | 1787 |
| | homocysteinemethyltransferase | |
| L70 | 5-methyltetrahydropteroyltriglutamate | 1788 |
| | homocysteinemethyltransferase | |
| L71 | 5′-nucleotidase | 1789 |
| L72 | 5′-nucleotidase | 1790 |
| L73 | 5′-nucleotidase | 1791 |
| L74 | 5′-nucleotidase | 1792 |
| L75 | 704aa long hypothetical glycosyltransferase | 1793 |
| L76 | 704aa long hypothetical glycosyltransferase | 1794 |
| L77 | 80 kDa nuclear cap binding protein | 1795 |
| L78 | 80 kDa nuclear cap binding protein | 1796 |
| L79 | 80 kDa nuclear cap binding protein | 1797 |
| L80 | 80 kDa nuclear cap binding protein | 1798 |
| L81 | Acetaldehyde dehydrogenase (acylating) | 1799 |
| L82 | Acetaldehyde dehydrogenase (acylating) | 1800 |
| L83 | Acetolactate synthase isozyme III small subunit | 1801 |
| L84 | Acetylcholine receptor protein, alpha chain | 1802 |
| L85 | Acetylcholine receptor protein, beta chain | 1803 |
| L86 | Aconitate hydratase 2 | 1804 |
| L87 | Aconitate hydratase 2 | 1805 |
| L88 | Aconitate hydratase 2 | 1806 |
| L89 | Aconitate hydratase 2 | 1807 |
| L90 | Aconitate hydratase 2 | 1808 |
| L91 | Acriflavine resistance protein B | DWY |
| L92 | Acriflavine resistance protein B | GGS |
| L93 | Acriflavine resistance protein B | IDQ |
| L94 | Acriflavine resistance protein B | NKV |
| L95 | Acriflavine resistance protein B | SEA |
| L96 | Acriflavine resistance protein B | 1809 |
| L97 | Acriflavine resistance protein B | 1810 |
| L98 | Acriflavine resistance protein B | 1811 |
| L99 | Acriflavine resistance protein B | 1812 |
| L100 | Acriflavine resistance protein B | 1813 |
| L101 | Acriflavine resistance protein B | 1814 |
| L102 | Acriflavine resistance protein B | 1815 |
| L103 | Acriflavine resistance protein B | 1816 |
| L104 | Acriflavine resistance protein B | 1817 |
| L105 | Acriflavine resistance protein B | 1818 |
| L106 | Acriflavine resistance protein B | 1819 |
| L107 | Acriflavine resistance protein B | 1820 |
| L108 | Acriflavine resistance protein B | 1821 |
| L109 | Acriflavine resistance protein B | 1822 |
| L110 | Acriflavine resistance protein B | 1823 |
| L111 | Acriflavine resistance protein B | 1824 |
| L112 | Acriflavine resistance protein B | 1825 |
| L113 | Acriflavine resistance protein B | 1826 |
| L114 | Acriflavine resistance protein B | 1827 |
| L115 | Acriflavine resistance protein B | 1828 |
| L116 | Acriflavine resistance protein B | 1829 |
| L117 | Acriflavine resistance protein B | 1830 |
| L118 | Acriflavine resistance protein B | 1831 |
| L119 | Acriflavine resistance protein B | 1832 |
| L120 | Acyl-CoA thioesterase II | 1833 |
| L121 | Acyl-CoA thioesterase II | 1834 |
| L122 | Acvl-CoA thioesterase II | 1835 |
| L123 | Acvl-CoA thioesterase II | 1836 |
| L124 | Acyl-CoA thioesterase II | 1837 |
| L125 | Acyl-coenzyme A thioesterase 4 | 1838 |
| L126 | Acyl-coenzyme A thioesterase 4 | 1839 |
| L127 | Acvl-coenzyme A thioesterase 4 | 1840 |
| L128 | Acyl-coenzyme A thioesterase 4 | 1841 |
| L129 | Acyl-coenzyme A thioesterase 4 | 1842 |
| L130 | Adenine glycosylase | 1843 |
| L131 | Adenylate cyclase | 1844 |
| L132 | Aerolysin | 1845 |
| L133 | Aerolysin | 1846 |
| L134 | Agglutinin | DWK |
| L135 | Agglutinin isolectin 1 | 1847 |
| L136 | Agglutinin isolectin 1 | 1848 |
| L137 | Aldehyde ferredoxin oxidoreductase | 1849 |
| L138 | Aldehyde oxidoreductase | 1850 |
| L139 | Aldehyde oxidoreductase | 1851 |
| L140 | Aldehyde oxidoreductase | 1852 |
| L141 | Aldehyde oxidoreductase | 1853 |
| L142 | Aldehyde oxidoreductase | 1854 |
| L143 | Alkyl hydroperoxide reductase subunit F | 1855 |
| L144 | Alkyl hydroperoxide reductase subunit F | 1856 |
| L145 | Alkyl hydroperoxide reductase subunit F | 1857 |
| L146 | Alkyl hydroperoxide reductase subunit F | 1858 |
| L147 | Alkyl hydroperoxide reductase subunit F | 1859 |
| L148 | Alkyl hydroperoxide reductase subunit F | 1860 |
| L149 | Alkyl hydroperoxide reductase subunit F | 1861 |
| L150 | Alkyl hydroperoxide reductase subunit F | 1862 |
| L151 | Alkyl hydroperoxide reductase subunit F | 1863 |
| L152 | Alkyl hydroperoxide reductase subunit F | 1864 |
| L153 | Allantoicase | 1865 |
| L154 | Allantoicase | 1866 |
| L155 | Alliin lyase 1 | SAV |
| L156 | Alliin lyase 1 | 1867 |
| L157 | Alliin lyase 1 | 1868 |
| L158 | Alliin lyase 1 | 186 |
| L159 | Alliin lyase 1 | 1870 |
| L160 | Alpha amylase | 1871 |
| L161 | Alpha amylase | 1872 |
| L162 | Alpha-actinin 1 | 1873 |
| L163 | Alpha-actinin 1 | 187 |
| L164 | Alpha-adaptin C | 1875 |
| L165 | Alpha-amylase | 1876 |
| L166 | Alpha-glucuronidase | LSD |
| L167 | Alpha-glucuronidase | 1877 |
| L168 | Alpha-glucuronidase | 1878 |
| L169 | Alpha-glucuronidase | 1879 |
| L170 | Alpha-glucuronidase | 1880 |
| L171 | Alpha-glucuronidase | 1881 |
| L172 | Alpha-glucuronidase | 1882 |
| L173 | Alpha-glucuronidase | 1883 |
| L174 | Alpha-glucuronidase | 1884 |
| L175 | Alpha-glucuronidase | 1885 |
| L176 | Alpha-glucuronidase | 1886 |
| L177 | Alpha-glucuronidase | 1887 |
| L178 | Alpha-glucuronidase | 1888 |
| L179 | Alpha-glucuronidase | 1889 |
| L180 | Alpha-glucuronidase | 1890 |
| L181 | Alpha-glucuronidase | 1891 |
| L182 | Alpha-glucuronidase | 1892 |
| L183 | Alpha-glucuronidase | 1893 |
| L184 | Alpha-glucuronidase | 1894 |
| L185 | Alpha-glucuronidase | 1895 |
| L186 | Alpha-glucuronidase | 1896 |
| L187 | Alpha-glucuronidase | 1897 |
| L188 | Alpha-L-arabinofuranosidase B | 1898 |
| L189 | Alpha-mannosidase | 1899 |
| L190 | Alr2269 protein | 1900 |
| L191 | AMP nucleosidase | 1901 |
| L192 | AMP nucleosidase | 1902 |
| L193 | AMP nucleosidase | 1903 |
| L194 | Angiopoietin-1 receptor | DAG |
| L195 | Angiopoietin-1 receptor | NSG |
| L196 | Angiopoietin-1 receptor | TSA |
| L197 | Angiopoietin-1 receptor | VPR |
| L198 | Angiopoietin-1 receptor | 1904 |
| L199 | Angiopoietin-1 receptor | 1905 |
| L200 | Angiopoietin-1 receptor | 1906 |
| L201 | Angiopoietin-1 receptor | 1907 |
| L202 | Angiopoietin-1 receptor | 1908 |
| L203 | Angiopoietin-1 receptor | 1909 |
| L204 | Angiopoietin-1 receptor | 1910 |
| L205 | Angiopoietin-1 receptor | 1911 |
| L206 | Angiopoietin-1 receptor | 1912 |
| L207 | Angiopoietin-1 receptor | 1913 |
| L208 | Angiopoietin-1 receptor | 1914 |
| L209 | Angiopoietin-1 receptor | 1915 |
| L210 | Angiopoietin-1 receptor | 1916 |
| L211 | Angiopoietin-1 receptor | 1917 |
| L212 | Angiopoietin-1 receptor | 1918 |
| L213 | Angiopoietin-1 receptor | 1919 |
| L214 | Angiopoietin-1 receptor | 1920 |
| L215 | Angiopoietin-1 receptor | 1921 |
| L216 | Angiopoietin-1 receptor | 1922 |
| L217 | Angiopoietin-1 receptor | 1923 |
| L218 | Angiopoietin-1 receptor | 1924 |
| L219 | Annexin A2 | QNK |
| L220 | Annexin A2 | 1925 |
| L221 | Annexin A2 | 1926 |
| L222 | Anthranilate phosphoribosyltransferase | 1927 |
| L223 | AP-2 complex subunit beta-2 | 1928 |
| L224 | Archaeosine tRNA-guanine transglycosylase | LGI |
| L225 | Archaeosine tRNA-guanine transglycosylase | 1929 |
| L226 | Archaeosine tRNA-guanine transglycosylase | 1930 |
| L227 | Archaeosine tRNA-guanine transglycosylase | 1931 |
| L228 | Archaeosine tRNA-guanine transglycosylase | 1932 |
| L229 | Archaeosine tRNA-guanine transglycosylase | 1933 |
| L230 | Archaeosine tRNA-guanine transglycosylase | 1934 |
| L231 | Archaeosine tRNA-guanine transglycosylase | 1935 |
| L232 | Archeal exosome RNA binding protein rrp4 | 1936 |
| L233 | Archeal exosome RNA binding protein rrp4 | 1937 |
| L234 | Archeal exosome RNA binding protein rrp4 | 1938 |
| L235 | Arginyl-tRNA synthetase | IDY |
| L236 | Arginyl-tRNA synthetase | 1939 |
| L237 | Arginyl-tRNA synthetase | 1940 |
| L238 | Arginyl-tRNA synthetase | 1941 |
| L239 | Arrestin | 1942 |
| L240 | Arrestin | 1943 |
| L241 | Arsenite oxidase | 1944 |
| L242 | Artificial linker | PGS |
| L243 | Artificial linker | ATK |
| L24 | Artificial linker | ASK |
| L245 | Artificial linker | 1945 |
| L246 | Artificial linker | 1946 |
| L247 | Artificial linker | 1947 |
| L248 | Artificial linker | 1948 |
| L249 | Artificial linker | 1949 |
| L250 | Artificial linker | 1950 |
| L251 | ATP phosphoribosyltransferase | ANR |
| L252 | ATP-dependent DNA helicase | YDP |
| L253 | ATP-dependent DNA helicase | 1951 |
| L254 | ATP-dependent DNA helicase | 1952 |
| L255 | ATP-dependent DNA helicase | 1953 |
| L256 | ATP-dependent DNA helicase | 1954 |
| L257 | ATP-dependent DNA helicase | 1955 |
| L258 | ATP-dependent DNA helicase | 1956 |
| L259 | ATP-dependent DNA helicase | 1957 |
| L260 | ATP-dependent DNA helicase | 1958 |
| L261 | AT-rich DNA-binding protein | 1959 |
| L262 | AT-rich DNA-binding protein | 1960 |
| L263 | Axonin-1 | DEG |
| L264 | Axonin-1 | ECF |
| L265 | Axonin-1 | 1961 |
| L266 | Axonin-1 | 1962 |
| L267 | Axonin-1 | 1963 |
| L268 | Axonin-1 | 1964 |
| L269 | Axonin-1 | 1965 |
| L270 | Axonin-1 | 1966 |
| L271 | Axonin-1 | 1967 |
| L272 | Bacilysin biosynthesis protein BacB | 1968 |
| L273 | Bacilysin biosynthesis protein BacB | 1969 |
| L274 | Bacilysin biosynthesis protein BacB | 1970 |
| L275 | Bacilysin biosynthesis protein BacB | 1971 |
| L276 | Bacilysin biosynthesis protein BacB | 1972 |
| L277 | Bacteriophage Mu transposase | 1973 |
| L278 | Bacteriophage Mu transposase | 1974 |
| L279 | Benzoyl-CoA-dihydrodiol lyase | 1975 |
| L280 | Benzoyl-CoA-dihydrodiol lyase | 1976 |
| L281 | Benzoyl-CoA-dihydrodiol lyase | 1977 |
| L282 | Benzoyl-CoA-dihydrodiol lyase | 1978 |
| L283 | Benzoyl-CoA-dihydrodiol lyase | 1979 |
| L284 | Benzoylformate decarboxylase | 1980 |
| L285 | Benzoylformate decarboxylase | 1981 |
| L286 | Benzoylformate decarboxylase | 1982 |
| L287 | Beta-amylase | 1983 |
| L288 | Beta-galactosidase | AIS |
| L289 | Beta-galactosidase | 1984 |
| L290 | Beta-galactosidase | 1985 |
| L291 | Beta-galactosidase | 1986 |
| L292 | Beta-galactosidase | 1987 |
| L293 | Beta-galactosidase | 1988 |
| L294 | Beta-galactosidase | 1989 |
| L295 | Beta-galactosidase | 1990 |
| L296 | Beta-galactosidase | 1991 |
| L297 | Beta-galactosidase | 1992 |
| L298 | Beta-galactosidase | 1993 |
| L299 | Beta-galactosidase | 1994 |
| L300 | Beta-galactosidase | 1995 |
| L301 | Beta-galactosidase | 1996 |
| L302 | Beta-galactosidase | 1997 |
| L303 | Beta-galactosidase | 1998 |
| L304 | Beta-galactosidase | 1999 |
| L305 | Beta-galactosidase | 2000 |
| L306 | Beta-galactosidase | 2001 |
| L307 | Beta-galactosidase | 2002 |
| L308 | Beta-galactosidase | 2003 |
| L309 | Beta-galactosidase | 2004 |
| L310 | Beta-galactosidase | 2005 |
| L311 | Beta-N-acetylhexosaminidase | QRE |
| L312 | Beta-N-acetylhexosaminidase | 2006 |
| L313 | Beta-N-acetylhexosaminidase | 2007 |
| L314 | Beta-N-acetylhexosaminidase | 2008 |
| L315 | Bifunctional NMN | 2009 |
| | adenylyltransferase/Nudix hydrolase | |
| L316 | Bifunctional purine biosynthesis | 2010 |
| | protein PURH | |
| L317 | Biliverdin reductase A | EHV |
| L318 | Biliverdin reductase A | LME |
| L319 | Biliverdin reductase A | 2011 |
| L320 | Biliverdin reductase A | 2012 |
| L321 | Biodegradative arginine decarboxylase | TVQ |
| L322 | Biodegradative arginine decarboxylase | 2013 |
| L323 | Biodegradative arginine decarboxylase | 2014 |
| L324 | Biodegradative arginine decarboxylase | 2015 |
| L325 | Biodegradative arginine decarboxylase | 2016 |
| L326 | Biodegradative arginine decarboxylase | 2017 |
| L327 | Biodegradative arginine decarboxylase | 2018 |
| L328 | Biodegradative arginine decarboxylase | 2019 |
| L329 | Biodegradative arginine decarboxylase | 2020 |
| L330 | Biodegradative arginine decarboxylase | 2021 |
| L331 | Biodegradative arginine decarboxylase | 2022 |
| L332 | Biodegradative arginine decarboxylase | 2023 |
| L333 | Biodegradative arginine decarboxylase | 2024 |
| L334 | Biotin carboxylase | 2025 |
| L335 | Bowman-Birk trypsin inhibitor | 2026 |
| L336 | Bpt4 gene 59 helicase assembly protein | KQI |
| L337 | BRCA1-associated RING domain protein 1 | 2027 |
| L338 | BRCA1-associated RING domain protein 1 | 2028 |
| L339 | BRCA1-associated RING domain protein 1 | 2029 |
| L340 | Breast cancer 2 | 2030 |
| L341 | Breast cancer 2 | 2031 |
| L342 | Breast cancer 2 | 2032 |
| L343 | Breast cancer 2 | 2033 |
| L344 | Breast cancer 2 | 2034 |
| L345 | Breast cancer 2 | 2035 |
| L346 | Butyrate response factor 2 | 2036 |
| L347 | C4b-binding protein | YKR |
| L348 | C4b-binding protein | 2037 |
| L349 | C5a peptidase | 2038 |
| L350 | C5a peptidase | 2039 |
| L351 | C5a peptidase | 2040 |
| L352 | C5a peptidase | 2041 |
| L353 | C5a peptidase | 2042 |
| L354 | C5a peptidase | 2043 |
| L355 | C5a peptidase | 2044 |
| L356 | C5a peptidase | 2045 |
| L357 | C5a peptidase | 2046 |
| L358 | C5a peptidase | 2047 |
| L359 | C5a peptidase | 2048 |
| L360 | C5a peptidase | 2049 |
| L361 | C5a peptidase | 2050 |
| L362 | Calcium-binding protein | 2051 |
| L363 | CarA | 2052 |
| L364 | CarA | 2053 |
| L365 | Carbamoyl phosphate synthetase (small chain) | 2054 |
| L366 | Carbamoyl phosphate synthetase (small chain) | 2055 |
| L367 | Carbamoyl phosphate synthetase (small chain) | 2056 |
| L368 | Carbamoyl phosphate synthetase (small chain) | 2057 |
| L369 | Carbamovl phosphate synthetase (small chain) | 2058 |
| L370 | Carbon monoxide dehydrogenase/ | 2059 |
| | acetyl-CoA synthase subunitalpha | |
| L371 | Carboxypeptidase Gp180 residues 503-882 | HRG |
| L372 | Catabolite activation-like protein | 2060 |
| L373 | Catabolite activation-like protein | 2061 |
| L374 | Catechol 2,3-dioxygenase | 2062 |
| L375 | Cation-independent mannose | 2063 |
| | 6-phosphate receptor | |
| L376 | CD3 epsilon and gamma ectodomain | 2064 |
| | fragment complex | |
| L377 | CD3 epsilon and gamma ectodomain | 2065 |
| | fragment complex | |
| L378 | Cell filamentation protein | SNP |
| L379 | Cell filamentation protein | 2066 |
| L380 | Cell filamentation protein | 2067 |
| L381 | Cellular coagulation factor XIII zymogen | DIT |
| L382 | Cellular coagulation factor XIII zymogen | NSD |
| L383 | Cellular coagulation factor XIII zymogen | TDT |
| L384 | Cellular coagulation factor XIII zymogen | 2068 |
| L385 | Cellular coagulation factor XIII zymogen | 2069 |
| L386 | Cellular coagulation factor XIII zymogen | 2070 |
| L387 | Cellular coagulation factor XIII zymogen | 2071 |
| L388 | Cellular coagulation factor XIII zymogen | 2072 |
| L389 | Cellular coagulation factor XIII zymogen | 2073 |
| L390 | Cellular coagulation factor XIII zymogen | 2074 |
| L391 | Cellular coagulation factor XIII zymogen | 2075 |
| L392 | Cellular coagulation factor XIII zymogen | 2076 |
| L393 | Cellular coagulation factor XIII zymogen | 2077 |
| L394 | Cellular coagulation factor XIII zymogen | 2078 |
| L395 | Cellular coagulation factor XIII zymogen | 2079 |
| L396 | Cellular coagulation factor XIII zymogen | 2080 |
| L397 | Cellular coagulation factor XIII zymogen | 2081 |
| L398 | Cellular coagulation factor XIII zymogen | 2082 |
| L399 | Cellular coagulation factor XIII zymogen | 2083 |
| L400 | Cellular coagulation factor XIII zymogen | 2084 |
| L401 | Cellular coagulation factor XIII zymogen | 2085 |
| L402 | Cellular coagulation factor XIII zymogen | 2086 |
| L403 | Cellulase | 2087 |
| L404 | Cellulase | 2088 |
| L405 | Cellulase | 2089 |
| L406 | Cellulase | 2090 |
| L407 | Cellulase | 2091 |
| L408 | Cellulase | 2092 |
| L409 | Cellulase | 2093 |
| L410 | Cellulase | 2094 |
| L411 | Cellulase | 2095 |
| L412 | Cellulase linker | 2096 |
| L413 | Cellulase linker | 2097 |
| L414 | Cellulase linker | 2098 |
| L415 | Cellulase linker | 2099 |
| L416 | Chaperone protein FimC | KLR |
| L417 | Chaperone protein FimC | QAA |
| L418 | Chaperone protein FimC | 2100 |
| L419 | Chaperone protein FimC | 2101 |
| L420 | Chaperone protein HscB | RHP |
| L421 | Chaperone protein HscB | 2102 |
| L422 | CheB methylesterase | 2103 |
| L423 | CheB methylesterase | 2104 |
| L424 | CheB methylesterase | 2105 |
| L425 | Chelatase, putative | 2106 |
| L426 | Chemotaxis receptor methyltransferase cheR | 2107 |
| L427 | Chemotaxis receptor methyltransferase cheR | 2108 |
| L428 | Chemotaxis receptor methyltransferase cheR | 2109 |
| L429 | Cholesterol oxidase | 2110 |
| L430 | Cholesterol oxidase | 2111 |
| L431 | Cholesterol oxidase | 2112 |
| L432 | Cholesterol oxidase | 2113 |
| L433 | Cholesterol oxidase | 2114 |
| L434 | Cholesterol oxidase | 2115 |
| L435 | Cholesterol oxidase | 2116 |
| L436 | Cholesterol oxidase | 2117 |
| L437 | Cholesterol oxidase | 2118 |
| L438 | Cholesterol oxidase | 2119 |
| L439 | Cholesterol oxidase | 2120 |
| L440 | Cholesterol oxidase | 2121 |
| L441 | Chromatin structure-remodeling | KNL |
| | complex protein RSC4 | |
| L442 | Chromatin structure-remodeling | 2122 |
| | complex protein RSC4 | |
| L443 | Chromatin structure-remodeling | 2123 |
| | complex protein RSC4 | |
| L444 | Chromatin structure-remodeling | 2124 |
| | complex protein RSC4 | |
| L445 | Chromodomain-helicase-DNA-binding protein 1 | 2125 |
| L446 | Chromodomain-helicase-DNA-binding protein 1 | 2126 |
| L447 | Cleavable disulfide | 2127 |
| L448 | Cleavable disulfide | 2128 |
| L449 | Cleavable disulfide | 2129 |
| L450 | Cleavable disulfide | 2130 |
| L451 | Cleavable disulfide | 2131 |
| L452 | Cleavable disulfide | 2132 |
| L453 | Cleavable disulfide | 2133 |
| L454 | Cleavable disulfide | 2134 |
| L455 | Cleavable disulfide | 2135 |
| L456 | Cleavable disulfide | 2136 |
| L457 | Cleavable disulfide | 2137 |
| L458 | Colicin Ia | 2138 |
| L459 | Collagen adhesin | 2139 |
| L460 | Complement C3 beta chain | 2140 |
| L461 | Complement C3 beta chain | 2141 |
| L462 | Complement C3 beta chain | 2142 |
| L463 | Complement C3 beta chain | 2143 |
| L464 | Complement decay-accelerating factor | EIY |
| L465 | Complement factor H | KRP |
| L466 | Complement receptor type 2 | 2144 |
| L467 | Conserved hypothetical protein | 2145 |
| L468 | Conserved hypothetical protein MTH1747 | DIR |
| L469 | Conserved hypothetical protein MTH1747 | 2146 |
| L470 | Conserved hypothetical protein MTH1747 | 2147 |
| L471 | Conserved hypothetical protein MTH1747 | 2148 |
| L472 | Conserved hypothetical protein MTH1747 | 2149 |
| L473 | Conserved hypothetical protein MTH1747 | 2150 |
| L474 | Conserved hypothetical protein MTH1747 | 2151 |
| L475 | Conserved hypothetical protein MTH1747 | 2152 |
| L476 | Conserved protein (MTH177) | 2153 |
| L477 | Creatine amidinohydrolase | 2154 |
| L478 | Cruciferin | 2155 |
| L479 | Cruciferin | 2156 |
| L480 | Cruciferin | 2157 |
| L481 | Cruciferin | 2158 |
| L482 | Cruciferin | 2159 |
| L483 | Cruciferin | 2160 |
| L484 | Cruciferin | 2161 |
| L485 | CSL3 | 2162 |
| L486 | CSL3 | 2163 |
| L487 | CTP synthase | 2164 |
| L488 | CTP synthase | 2165 |
| L489 | Cullin homolog | HKN |
| L490 | Cullin homolog | 2166 |
| L491 | Cullin homolog | 2167 |
| L492 | Cullin homolog | 2168 |
| L493 | Cullin homolog | 2169 |
| L494 | Cullin homolog | 2170 |
| L495 | Cyclin A2 | 2171 |
| L496 | Cysteine-rich secretory protein | 2172 |
| L497 | Cytidine deaminase | 2173 |
| L498 | Cytidine deaminase | 2174 |
| L499 | Cytidine deaminase | 2175 |
| L500 | Cytochrome b-c1 complex subunit | 2176 |
| | Rieske, mitochondrial | |
| L501 | Cytochrome c oxidase subunit 2 | QAV |
| L502 | Cytochrome c oxidase subunit 2 | 2177 |
| L503 | Cytochrome c oxidase subunit 2 | 2178 |
| L504 | Cytochrome c oxidase subunit 2 | 2179 |
| L505 | Cytochrome c oxidase subunit 2 | 2180 |
| L506 | Cytochrome c4 | GGK |
| L507 | Cytochrome c4 | QGM |
| L508 | D-aminopeptidase | 2181 |
| L509 | DDMC | 2182 |
| L510 | DDMC | 2183 |
| L511 | Deltex protein | 2184 |
| L512 | Deoxyuridine 5′-triphosphate nucleotidohydrolase | 2185 |
| L513 | Diaminopimelate epimerase | 2186 |
| L514 | Diaminopimelate epimerase | 2187 |
| L515 | Diaminopimelate epimerase | 2188 |
| L516 | Di-heme peroxidase | SGC |
| L517 | Di-heme peroxidase | 2189 |
| L518 | Dihydropyrimidine dehydrogenase | 2190 |
| L519 | Dihydropyrimidine dehydrogenase | 2191 |
| L520 | Dihydropyrimidine dehydrogenase | 2192 |
| L521 | Dihydropyrimidine dehydrogenase | 2193 |
| L522 | Dihydropyrimidine dehydrogenase | 2194 |
| L523 | Dihydropyrimidine dehydrogenase | 2195 |
| L524 | Dihydropyrimidine dehydrogenase | 2196 |
| L525 | Dihydropyrimidine dehydrogenase | 2197 |
| L526 | Dihydropyrimidine dehydrogenase | 2198 |
| L527 | Dihydropyrimidine dehydrogenase | 2199 |
| L528 | Dihydropyrimidine dehydrogenase | 2200 |
| L529 | Dihydropyrimidine dehydrogenase | 2201 |
| L530 | Dihydropyrimidine dehydrogenase | 2202 |
| L531 | Dihydropyrimidine dehydrogenase | 2203 |
| L532 | Dihydropyrimidine dehydrogenase | 2204 |
| L533 | Dihydropyrimidine dehydrogenase | 2205 |
| L534 | Dihydropyrimidine dehydrogenase | 2206 |
| L535 | Dihydropyrimidine dehydrogenase | 2207 |
| L536 | Dihydropyrimidine dehydrogenase | 2208 |
| L537 | Dihydropyrimidine dehydrogenase | 2209 |
| L538 | Dihydropyrimidine dehydrogenase | 2210 |
| L539 | Dihydropyrimidine dehydrogenase | 2211 |
| L540 | Dihydropyrimidine dehydrogenase | 2212 |
| L541 | Dihydropyrimidine dehydrogenase | 2213 |
| L542 | Dihydropyrimidine dehydrogenase | 2214 |
| L543 | Dihydropyrimidine dehydrogenase | 2215 |
| L544 | Dihydropyrimidine dehydrogenase | 2216 |
| L545 | Dihydropyrimidine dehydrogenase | 2217 |
| L546 | Dihydropyrimidine dehydrogenase | 2218 |
| L547 | Dihydropyrimidine dehydrogenase | 2219 |
| L548 | Dihydropyrimidine dehydrogenase | 2220 |
| L549 | Discoidin-1 subunit A | 2221 |
| L550 | Discoidin-1 subunit A | 2222 |
| L551 | Discoidin-1 subunit A | 2223 |
| L552 | Dissimilatory copper-containing nitritereductase | 2224 |
| L553 | D-lactate dehydrogenase | DTF |
| L554 | D-lactate dehydrogenase | 2225 |
| L555 | D-lactate dehydrogenase | 2226 |
| L556 | D-lactate dehydrogenase | 2227 |
| L557 | D-lactate dehydrogenase | 2228 |
| L558 | D-lactate dehydrogenase | 2229 |
| L559 | D-lactate dehydrogenase | 2230 |
| L560 | DNA damage-binding protein 1 | LCA |
| L561 | DNA damage-binding protein 1 | 2231 |
| L562 | DNA damage-binding protein 1 | 2232 |
| L563 | DNA damage-binding protein 1 | 2233 |
| L564 | DNA damage-binding protein 1 | 2234 |
| L565 | DNA damage-binding protein 1 | 2235 |
| L566 | DNA damage-binding protein 1 | 2236 |
| L567 | DNA damage-binding protein 1 | 2237 |
| L568 | DNA damage-binding protein 1 | 2238 |
| L569 | DNA damage-binding protein 1 | 2239 |
| L570 | DNA damage-binding protein 1 | 2240 |
| L571 | DNA damage-binding protein 1 | 2241 |
| L572 | DNA damage-binding protein 1 | 2242 |
| L573 | DNA damage-binding protein 1 | 2243 |
| L574 | DNA damage-binding protein 1 | 2244 |
| L575 | DNA damage-binding protein 1 | 2245 |
| L576 | DNA damage-binding protein 1 | 2246 |
| L577 | DNA damage-binding protein 1 | 2247 |
| L578 | DNA damage-binding protein 1 | 2248 |
| L579 | DNA damage-binding protein 1 | 2249 |
| L580 | DNA damage-binding protein 1 | 2250 |
| L581 | DNA damage-binding protein 1 | 2251 |
| L582 | DNA damage-binding protein 1 | 2252 |
| L583 | DNA gyrase B | ALS |
| L584 | DNA gyrase B | 2253 |
| L585 | DNA gyrase B | 2254 |
| L586 | DNA gyrase B | 2255 |
| L587 | DNA gyrase B | 2256 |
| L588 | DNA gyrase B | 2257 |
| L589 | DNA gyrase B | 2258 |
| L590 | DNA gyrase B | 2259 |
| L591 | DNA gyrase B | 2260 |
| L592 | DNA gyrase B | 2261 |
| L593 | DNA gyrase B | 2262 |
| L594 | DNA gyrase B | 2263 |
| L595 | DNA ligase | 2264 |
| L596 | DNA ligase | 2265 |
| L597 | DNA ligase | 2266 |
| L598 | DNA ligase | 2267 |
| L599 | DNA ligase | 2268 |
| L600 | DNA mismatch repair protein MutS | MDA |
| L601 | DNA mismatch repair protein MutS | SII |
| L602 | DNA mismatch repair protein MutS | 2269 |
| L603 | DNA mismatch repair protein MutS | 2270 |
| L604 | DNA mismatch repair protein MutS | 2271 |
| L605 | DNA mismatch repair protein MutS | 2272 |
| L606 | DNA mismatch repair protein MutS | 2273 |
| L607 | DNA polymerase | FSP |
| L608 | DNA polymerase | RQF |
| L609 | DNA polymerase | 2274 |
| L610 | DNA polymerase | 2275 |
| L611 | DNA polymerase | 2276 |
| L612 | DNA polymerase | 2277 |
| L613 | DNA polymerase | 2278 |
| L614 | DNA polymerase | 2279 |
| L615 | DNA polymerase | 2280 |
| L616 | DNA polymerase | 2281 |
| L617 | DNA polymerase alpha subunit B | 2282 |
| L618 | DNA polymerase alpha subunit B | 2283 |
| L619 | DNA polymerase alpha subunit B | 2284 |
| L620 | DNA polymerase alpha subunit B | 2285 |
| L621 | DNA polymerase alpha subunit B | 2286 |
| L622 | DNA polymerase alpha subunit B | 2287 |
| L623 | DNA polymerase alpha subunit B | 2288 |
| L624 | DNA polymerase alpha subunit B | 2289 |
| L625 | DNA polymerase alpha subunit B | 2290 |
| L626 | DNA polymerase alpha subunit B | 2291 |
| L627 | DNA polymerase eta | ALS |
| L628 | DNA polymerase eta | 2292 |
| L629 | DNA polymerase eta | 2293 |
| L630 | DNA polymerase eta | 2294 |
| L631 | DNA polymerase eta | 2295 |
| L632 | DNA polymerase eta | 2296 |
| L633 | DNA polymerase I | AGV |
| L634 | DNA polymerase I | ELE |
| L635 | DNA polymerase I | 2297 |
| L636 | DNA primase | DHK |
| L637 | DNA primase | 2298 |
| L638 | DNA primase | 2299 |
| L639 | DNA primase | 2300 |
| L640 | DNA primase | 2301 |
| L641 | DNA primase | 2302 |
| L642 | DNA primase | 2303 |
| L643 | DNA primase | 2304 |
| L644 | DNA primase/helicase | AGY |
| L645 | DNA primase/helicase | 2305 |
| L646 | DNA primase/helicase | 2306 |
| L647 | DNA primase/helicase | 2307 |
| L648 | DNA primase/helicase | 2308 |
| L649 | DNA primase/helicase | 2309 |
| L650 | DNA primase/helicase | 2310 |
| L651 | DNA primase/helicase | 2311 |
| L652 | DNA primase/helicase | 2312 |
| L653 | DNA primase/helicase | 2313 |
| L654 | DNA primase/helicase | 2314 |
| L655 | DNA topoisomerase 2 | EES |
| L656 | DNA topoisomerase 2 | IPI |
| L657 | DNA topoisomerase 2 | KEL |
| L658 | DNA topoisomerase 2 | 2315 |
| L659 | DNA topoisomerase 2 | 2316 |
| L660 | DNA topoisomerase 2 | 2317 |
| L661 | DNA topoisomerase 2 | 2318 |
| L662 | DNA topoisomerase 2 | 2319 |
| L663 | DNA topoisomerase 2 | 2320 |
| L664 | DNA topoisomerase 2 | 2321 |
| L665 | DNA topoisomerase 2 | 2322 |
| L666 | DNA topoisomerase 2 | 2323 |
| L667 | DNA topoisomerase I | 2324 |
| L668 | DNA topoisomerase I | 2325 |
| L669 | DNA topoisomerase I | 2326 |
| L670 | DNA topoisomerase II, alpha isozyme | PDL |
| L671 | DNA topoisomerase II, alpha isozyme | 2327 |
| L672 | DNA topoisomerase II, alpha isozyme | 2328 |
| L673 | DNA topoisomerase II, alpha isozyme | 2329 |
| L674 | DNA topoisomerase II, alpha isozyme | 2330 |
| L675 | DNA topoisomerase II, alpha isozyme | 2331 |
| L676 | DNA topoisomerase II, alpha isozyme | 2332 |
| L677 | DNA topoisomerase II, alpha isozyme | 2333 |
| L678 | DNA topoisomerase II, alpha isozyme | 2334 |
| L679 | DNA topoisomerase VI A subunit | 2335 |
| L680 | DNA topoisomerase VI A subunit | 2336 |
| L681 | DNA topoisomerase VI A subunit | 2337 |
| L682 | DNA topoisomerase VI A subunit | 2338 |
| L683 | DNA topoisomerase VI A subunit | 2339 |
| L684 | DNA topoisomerase VI A subunit | 2340 |
| L685 | DNA-3-methyladenine glycosylase 2 | 2341 |
| L686 | DNA-binding response regulator MtrA | 2342 |
| L687 | DNA-directed RNA polymerase beta chain | 2343 |
| L688 | DNA-directed RNA polymerase beta chain | 2344 |
| L689 | DNA-directed RNA polymerase beta chain | 2345 |
| L690 | DNA-directed RNA polymerase beta chain | 2346 |
| L691 | DNA-directed RNA polymerase beta chain | 2347 |
| L692 | DNA-directed RNA polymerase beta chain | 2348 |
| L693 | DNA-directed RNA polymerase beta chain | 2349 |
| L694 | DNA-directed RNA polymerase beta chain | 2350 |
| L695 | DNA-directed RNA polymerase | 2351 |
| | II 14.2 kDa polypeptide | |
| L696 | DNA-directed RNA polymerase | 2352 |
| | II 14.2 kDa polypeptide | |
| L697 | DNA-directed RNA polymerase, | 2353 |
| | subunit E′ (rpoe1) | |
| L698 | DNA-directed RNA polymerase, | 2354 |
| | subunit E′ (rpoe1) | |
| L699 | DNA-directed RNA polymerases | ITP |
| | I, II, and III 27 kDa polypeptide | |
| L700 | DNA-directed RNA polymerases | 2355 |
| | I, II, and III 27 kDa polypeptide | |
| L701 | DNA-directed RNA polymerases | 2356 |
| | I, II, and III 27 kDa polypeptide | |
| L702 | DNA-directed RNA polymerases | 2357 |
| | I, II, and III 27 kDa polypeptide | |
| L703 | DNA-directed RNA polymerases | 2358 |
| | I, II, and III 27 kDa polypeptide | |
| L704 | Drosophila neuroglian | 2359 |
| L705 | Dystroglycan | 2360 |
| L706 | Dystrophin | 2361 |
| L707 | Dystrophin | 2362 |
| L708 | Dystrophin | 2363 |
| L709 | Dystrophin | 2364 |
| L710 | Dystrophin | 2365 |
| L711 | Dystrophin | 2366 |
| L712 | Dystrophin | 2367 |
| L713 | E2A DNA-binding protein | 2368 |
| L714 | E2A DNA-binding protein | 2369 |
| L715 | E3 sumo-protein ligase SIZ1 | 2370 |
| L716 | E3 sumo-protein ligase SIZ1 | 2371 |
| L717 | E3 sumo-protein ligase SIZ1 | 2372 |
| L718 | Early switch protein xol-1 2.2 k splice form | 2373 |
| L719 | EGF-like module containing mucin-like | 2374 |
| | hormonereceptor-like 2 precursor | |
| L720 | EGF-like module containing mucin-like | 2375 |
| | hormonereceptor-like 2 precursor | |
| L721 | Elongation factor 1-gamma 1 | 2376 |
| L722 | Elongation factor 1-gamma l | 2377 |
| L723 | Elongation factor g | 2378 |
| L724 | Elongation factor G | 2379 |
| L725 | Elongation factor G | 2380 |
| L726 | Elongation factor G | 2381 |
| L727 | Elongation factor G | 2382 |
| L728 | Elongation factor G | 2383 |
| L729 | Elongation factor G | 2384 |
| L730 | Elongation factor G | 2385 |
| L731 | Elongation factor G | 2386 |
| L732 | Elongation factor G | 2387 |
| L733 | Elongation factor P | 2388 |
| L734 | Elongation factor Ts | 2389 |
| L735 | Elongation factor Ts | 2390 |
| L736 | Elongation factor Ts | 2391 |
| L737 | Elongation factor Tu (ef-Tu) | 2392 |
| L738 | Endoglucanase | 2393 |
| L739 | Endonuclease PI-SceI | 2394 |
| L740 | Endonuclease PI-SceI | 2395 |
| L741 | Endonuclease PI-SceI | 2396 |
| L742 | Endonuclease PI-SceI | 2397 |
| L743 | Endonuclease PI-SceI | 2398 |
| L744 | Endonuclease PI-SceI | 2399 |
| L745 | Endonuclease PI-SceI | 2400 |
| L746 | Endonuclease PI-SceI | 2401 |
| L747 | Endonuclease PI-SceI | 2402 |
| L748 | Enterobactin synthetase component F | 2403 |
| L749 | Enterobactin synthetase component F | 2404 |
| L750 | Enterobactin synthetase component F | 2405 |
| L751 | Enterobactin synthetase component F | 2406 |
| L752 | Enterobactin synthetase component F | 2407 |
| L753 | Enterobactin synthetase component F | 2408 |
| L754 | Enterobactin synthetase component F | 2409 |
| L755 | Enterobactin synthetase component F | 2410 |
| L756 | Enterobactin synthetase component F | 2411 |
| L757 | Enterochelin esterase | 2412 |
| L758 | Epo receptor | EVV |
| L759 | Epo receptor | 2413 |
| L760 | Erythrocyte binding antigen region II | 2414 |
| L761 | Erythrocyte binding antigen region II | 2415 |
| L762 | Erythrocyte binding antigen region II | 2416 |
| L763 | Erythrocyte binding antigen region II | 2417 |
| L764 | Erythrocyte binding antigen region II | 2418 |
| L765 | E-selectin | 2419 |
| L766 | Esterase EstA | SAP |
| L767 | Esterase EstA | 2420 |
| L768 | Esterase EstA | 2421 |
| L769 | Eukaryotic peptide chain release | 2422 |
| | factor GTP-binding subunit | |
| L770 | Exonuclease I | RQP |
| L771 | Exonuclease I | 2423 |
| L772 | FascIclIn I | SDP |
| L773 | FascIclIn I | 2424 |
| L774 | Fibrillin-1 | 2425 |
| L775 | Fibrillin-1 | 2426 |
| L776 | Fibrillin-1 | 2427 |
| L777 | Fibrillin-1 | 2428 |
| L778 | Fibrillin-1 | 2429 |
| L779 | Fibronectin | 2430 |
| L780 | Fibronectin | 2431 |
| L781 | Fibronectin | 2432 |
| L782 | Flagellar hook protein FlgE | 2433 |
| L783 | Flagellar hook protein FlgE | 2434 |
| L784 | Flagellar hook protein FlgE | 2435 |
| L785 | Flagellar hook protein FlgE | 2436 |
| L786 | Flagellar hook protein FlgE | 2437 |
| L787 | Flagellar hook protein FlgE | 2438 |
| L788 | Flagellar hook protein FlgE | 2439 |
| L789 | Flavohemoprotein | 2440 |
| L790 | Flexible G/S rich linker | G |
| L791 | Flexible G/S rich linker | S |
| L792 | Flexible G/S rich linker | GG |
| L793 | Flexible G/S rich linker | GS |
| L794 | Flexible G/S rich linker | GGS |
| L795 | Flexible G/S rich linker | GGG |
| L796 | Flexible G/S rich linker | 2441 |
| L797 | Flexible G/S rich linker | 2442 |
| L798 | Flexible G/S rich linker | 2443 |
| L799 | Flexible G/S rich linker | 2444 |
| L800 | Flexible G/S rich linker | 2445 |
| L801 | Flexible G/S rich linker | 2446 |
| L802 | Flexible G/S rich linker | 2447 |
| L803 | Flexible G/S rich linker | 2448 |
| L804 | Flexible G/S rich linker | 2449 |
| L805 | Flexible G/S rich linker | 2450 |
| L806 | Flexible G/S rich linker | 2451 |
| L807 | Flexible G/S rich linker | 2452 |
| L808 | Flexible G/S rich linker | 2453 |
| L809 | Flexible G/S rich linker | 2454 |
| L810 | Focal adhesion kinase 1 | 2455 |
| L811 | FolC bifunctional protein | 2456 |
| L812 | FolC bifunctional protein | 2457 |
| L813 | FolC bifunctional protein | 2458 |
| L814 | FolC bifunctional protein | 2459 |
| L815 | FolC bifunctional protein | 2460 |
| L816 | FolC bifunctional protein | 2461 |
| L817 | FolC bifunctional protein | 2462 |
| L818 | FolC bifunctional protein | 2463 |
| L819 | Follistatin | 2464 |
| L820 | Formate dehydrogenase (large subunit) | YDK |
| L821 | Formate dehydrogenase (large subunit) | 2465 |
| L822 | Formate dehydrogenase (large subunit) | 2466 |
| L823 | Formate dehydrogenase (large subunit) | 2467 |
| L824 | Formate dehydrogenase (large subunit) | 2468 |
| L825 | Formate dehydrogenase (large subunit) | 2469 |
| L826 | Formate dehydrogenase (large subunit) | 2470 |
| L827 | Formate dehydrogenase (large subunit) | 2471 |
| L828 | Formate dehydrogenase (large subunit) | 2472 |
| L829 | Formate dehydrogenase (large subunit) | 2473 |
| L830 | Formate dehydrogenase (large subunit) | 2474 |
| L831 | Formate dehydrogenase (large subunit) | 2475 |
| L832 | Formate dehydrogenase (large subunit) | 2476 |
| L833 | Formate dehydrogenase, | 2477 |
| | nitrate-inducible major subunit | |
| L834 | Formate dehydrogenase, | 2478 |
| | nitrate-inducible, major subunit | |
| L835 | Formate dehydrogenase, | 2479 |
| | nitrate-inducible, major subunit | |
| L836 | Formate dehydrogenase, | 2480 |
| | nitrate-inducible, major subunit | |
| L837 | Formate dehydrogenase, | 2481 |
| | nitrate-inducible, major subunit | |
| L838 | Formate dehydrogenase, | 2482 |
| | nitrate-inducible, major subunit | |
| L839 | Formate dehydrogenase, | 2483 |
| | nitrate-inducible, major subunit | |
| L840 | Formate dehydrogenase, | 2484 |
| | nitrate-inducible, major subunit | |
| L841 | Formate dehydrogenase, | 2485 |
| | nitrate-inducible, major subunit | |
| L842 | Formate dehydrogenase, | 2486 |
| | nitrate-inducible, major subunit | |
| L843 | Formate dehydrogenase, | 2487 |
| | nitrate-inducible, major subunit | |
| L844 | Formate dehydrogenase, | 2488 |
| | nitrate-inducible, major subunit | |
| L845 | Formate dehydrogenase, | 2489 |
| | nitrate-inducible, major subunit | |
| L846 | Formate dehydrogenase, | 2490 |
| | nitrate-inducible, major subunit | |
| L847 | Fumarylacetoacetate hydrolase | 2491 |
| L848 | Galactose oxidase | GSV |
| L849 | Galactose oxidase | GWK |
| L850 | Galactose oxidase | IAE |
| L851 | Galactose oxidase | KRQ |
| L852 | Galactose oxidase | QDT |
| L853 | Galactose oxidase | TPN |
| L854 | Galactose oxidase | 2492 |
| L855 | Galactose oxidase | 2493 |
| L856 | Galactose oxidase | 2494 |
| L857 | Galactose oxidase | 2495 |
| L858 | Galactose oxidase | 2496 |
| L859 | Galactose oxidase | 2497 |
| L860 | Galactose oxidase | 2498 |
| L861 | Galactose oxidase | 2499 |
| L862 | Galactose oxidase | 2500 |
| L863 | Galactose oxidase | 2501 |
| L864 | Galactose oxidase | 2502 |
| L865 | Galactose oxidase | 2503 |
| L866 | Galactose oxidase | 2504 |
| L867 | Galactose oxidase | 2505 |
| L868 | Galactose oxidase | 2506 |
| L869 | Galactose oxidase | 2507 |
| L870 | Galactose oxidase | 2508 |
| L871 | Galactose oxidase | 2509 |
| L872 | Galactose oxidase | 2510 |
| L873 | Galactose oxidase | 2511 |
| L874 | Galactose oxidase | 2512 |
| L875 | Galactose oxidase | 2513 |
| L876 | Galactose oxidase | 2514 |
| L877 | Galactose oxidase | 2515 |
| L878 | Gamma B-crystallin | 2516 |
| L879 | Gamma-delta T-cell receptor | 2517 |
| L880 | Gelation factor | DSS |
| L881 | Gelation factor | 2518 |
| L882 | Gelation factor | 2519 |
| L883 | Gelation factor | 2520 |
| L884 | Gene activator alpha | 2521 |
| L885 | Gingipain R | 2522 |
| L886 | Glucodextranase | 2523 |
| L887 | Glucodextranase | 2524 |
| L888 | Glucodextranase | 2525 |
| L889 | Glucosamine-fructose-6-phosphate aminotransferase | YEQ |
| L890 | Glucosamine-fructose-6-phosphate aminotransferase | 2526 |
| L891 | Glucosamine-fructose-6-phosphate aminotransferase | 2527 |
| L892 | Glucosamine-fructose-6-phosphate aminotransferase | 2528 |
| L893 | Glucosamine-fructose-6-phosphate aminotransferase | 2529 |
| L894 | Glucosamine-fructose-6-phosphate aminotransferase | 2530 |
| L895 | Glucosamine-fructose-6-phosphate aminotransferase | 2531 |
| L896 | Glucosamine-fructose-6-phosphate aminotransferase | 2532 |
| L897 | Glucosamine-fructose-6-phosphate aminotransferase | 2533 |
| L898 | Glucosamine-fructose-6-phosphate aminotransferase | 2534 |
| L899 | Glucosamine-fructose-6-phosphate aminotransferase | 2535 |
| L900 | Glucose-1-phosphate adenylyltransferase small subunit | 2536 |
| L901 | Glucose-1-phosphate adenylyltransferase small subunit | 2537 |
| L902 | Glucose-6-phosphate isomerase | KNA |
| L903 | Glucose-6-phosphate isomerase | VGF |
| L904 | Glucose-6-phosphate isomerase | 2538 |
| L905 | Glucose-6-phosphate isomerase | 2539 |
| L906 | Glucose-6-phosphate isomerase, conjectural | 2540 |
| L907 | Glutamate dehydrogenase | 2541 |
| L908 | Glutamate dehydrogenase | 2542 |
| L909 | Glutamate receptor interacting protein | 2543 |
| L910 | Glutamate synthase [NADPH] large chain | 2544 |
| L911 | Glutamate synthase [NADPH] large chain | 2545 |
| L912 | Glutamate synthase [NADPH] large chain | 2546 |
| L913 | Glutamate synthase [NADPH] large chain | 2547 |
| L914 | Glutamate synthase [NADPH] large chain | 2548 |
| L915 | Glutamate synthase [NADPH] large chain | 2549 |
| L916 | Glutamate synthase [NADPH] large chain | 2550 |
| L917 | Glutamine synthetase | 2551 |
| L918 | Glutamine synthetase | 2552 |
| L919 | Glutamyl-tRNA synthetase | 2553 |
| L920 | Glutamyl-tRNA synthetase | 2554 |
| L921 | Glutamyl-tRNA synthetase | 2555 |
| L922 | Glutamyl-tRNA synthetase | 2556 |
| L923 | Glutamyl-tRNA synthetase | 2557 |
| L924 | Glutamyl-tRNA synthetase | 2558 |
| L925 | Glutamyl-tRNA synthetase | 2559 |
| L926 | Glutamyl-tRNA synthetase | 2560 |
| L927 | Glutaredoxin 2 | 2561 |
| L928 | Glutathione S-transferase | 2562 |
| L929 | Glutathione S-transferase | 2563 |
| L930 | Glutathione S-transferase | 2564 |
| L931 | Glutathione S-transferase 1-6 | 2565 |
| L932 | Glutathione S-transferase A1 | 2566 |
| L933 | Glutathlone S-transferase I | NKP |
| L934 | GlutathIone S-transferase I | 2567 |
| L935 | Glutathione synthetase | 2568 |
| L936 | Glutathione transferase GST1-4 | 2569 |
| L937 | Glutathione transferase GST1-4 | 2570 |
| L938 | Glutathione transferase sigma class | 2571 |
| L939 | Glycerol-3-phosphate dehydrogenase [NAD(P)+] | 2572 |
| L940 | Glycine cleavage system | 2573 |
| | transcriptionalrepressor, putative | |
| L941 | Glycolipid-anchored surface protein 2 | 2574 |
| L942 | Glycolipid-anchored surface protein 2 | 2575 |
| L943 | Glycyl-tRNA synthetase | KFA |
| L944 | Glycyl-tRNA synthetase | 2576 |
| L945 | Glycyl-tRNA synthetase | 2577 |
| L946 | Glycyl-tRNA synthetase | 2578 |
| L947 | Glycyl-tRNA synthetase | 2579 |
| L948 | Glycyl-tRNA synthetase | 2580 |
| L949 | Glycyl-tRNA synthetase | 2581 |
| L950 | Glycyl-tRNA synthetase | 2582 |
| L951 | Glycyl-tRNA synthetase | 2583 |
| L952 | Glycyl-tRNA synthetase | 2584 |
| L953 | Growth hormone receptor | 2585 |
| L954 | Growth hormone receptor | 2586 |
| L955 | Harmonin | 2587 |
| L956 | HasR protein | 2588 |
| L957 | HasR protein | 2589 |
| L958 | Hemin transport protein HemS | 2590 |
| L959 | Hemin transport protein HemS | 2591 |
| L960 | Hemin transport protein HemS | 2592 |
| L961 | Hemoglobin | 2593 |
| L962 | Hemolytic lectin CEL-iii | 2594 |
| L963 | Hepatocyte nuclear factor 6 | 2595 |
| L964 | Histidyl-tRNA synthetase | 2596 |
| L965 | HNH homing endonuclease | 2597 |
| L966 | HNH homing endonuclease | 2598 |
| L967 | HNH homing endonuclease | 2599 |
| L968 | Homoserine dehydrogenase | 2600 |
| L969 | Homoserine kinase | 2601 |
| L970 | Homoserine kinase | 2602 |
| L971 | Homoserine kinase | 2603 |
| L972 | Homoserine kinase | 2604 |
| L973 | HTH-type transcriptional | 2605 |
| | regulator MqsA (Ygit/B3021) | |
| L974 | HTH-type transcriptional repressor YvoA | 2606 |
| L975 | HTH-type transcriptional repressor YvoA | 2607 |
| L976 | Human IgGI middle hinge linker | 2608 |
| L977 | Human IgG1 upper hinge linker | 2609 |
| L978 | Human IgG3 middle hinge linker | 2610 |
| L979 | Human IgG3m15 middle hinge linker | 2611 |
| L980 | Human IgG4 lower hinge linker | 2612 |
| L981 | Human IgG4 middle hinge linker | 2613 |
| L982 | Human IgG4 upper hinge linker | 2614 |
| L983 | Hybrid cluster protein | 2615 |
| L984 | Hybrid cluster protein | 2616 |
| L985 | Hybrid cluster protein | 2617 |
| L986 | Hybrid cluster protein | 2618 |
| L987 | Hybrid cluster protein | 2619 |
| L988 | Hypothetical conserved protein, GK1056 | 2620 |
| L989 | Hypothetical membrane spanning protein | 2621 |
| L990 | Hypothetical methylmalonyl-CoA | 2622 |
| | decarboxylase alpha subunit | |
| L991 | Hypothetical methylmalonyl-CoA | 2623 |
| | decarboxylase alpha subunit | |
| L992 | Hypothetical methylmalonyl-CoA | 2624 |
| | decarboxylase alpha subunit | |
| L993 | Hypothetical methylmalonyl-CoA | 2625 |
| | decarboxylase alpha subunit | |
| L994 | Hypothetical methylmalonyl-CoA | 2626 |
| | decarboxylase alpha subunit | |
| L995 | Hypothetical methylmalonyl-CoA | 2627 |
| | decarboxylase alpha subunit | |
| L996 | Hypothetical methylmalonyl-CoA | 2628 |
| | decarboxylase alpha subunit | |
| L997 | Hypothetical protein | AEP |
| L998 | Hypothetical protein | 2629 |
| L999 | Hypothetical protein APE0525 | PTL |
| L1000 | Hypothetical protein APE0525 | 2630 |
| L1001 | Hypothetical protein LOC449832 | 2631 |
| L1002 | Hypothetical protein LOC449832 | 2632 |
| L1003 | Hypothetical protein PA4388 | 2633 |
| L1004 | Hypothetical protein PA5201 | ASE |
| L1005 | Hypothetical protein PA5201 | QDP |
| L1006 | Hypothetical protein PA5201 | VKL |
| L1007 | Hypothetical protein PA5201 | 2634 |
| L1008 | Hypothetical protein PA5201 | 2635 |
| L1009 | Hypothetical protein PA5201 | 2636 |
| L1010 | Hypothetical protein PA5201 | 2637 |
| L1011 | Hypothetical protein PA5201 | 2638 |
| L1012 | Hypothetical protein PA5201 | 2639 |
| L1013 | Hypothetical protein PA5201 | 2640 |
| L1014 | Hypothetical protein PA5201 | 2641 |
| L1015 | Hypothetical protein PA5201 | 2642 |
| L1016 | Hypothetical protein PA5201 | 2643 |
| L1017 | Hypothetical protein PA5201 | 2644 |
| L1018 | Hypothetical protein PA5201 | 2645 |
| L1019 | Hypothetical protein PA5201 | 2646 |
| L1020 | Hypothetical protein PA5201 | 2647 |
| L1021 | Hypothetical protein PA5201 | 2648 |
| L1022 | Hypothetical protein PA5201 | 2649 |
| L1023 | Hypothetical protein PA5201 | 2650 |
| L1024 | Hypothetical protein PA5201 | 2651 |
| L1025 | Hypothetical protein PA5201 | 2652 |
| L1026 | Hypothetical protein PA5201 | 2653 |
| L1027 | Hypothetical protein PH0495 | ASN |
| L1028 | Hypothetical protein PH0495 | 2654 |
| L1029 | Hypothetical protein PH0495 | 2655 |
| L1030 | Hypothetical protein PH0495 | 2656 |
| L1031 | Hypothetical protein PH0495 | 2657 |
| L1032 | Hypothetical protein PH0510 | 2658 |
| L1033 | Hypothetical protein PH0510 | 2659 |
| L1034 | Hypothetical protein PH1313 | 2660 |
| L1035 | Hypothetical protein PH1313 | 2661 |
| L1036 | Hypothetical protein SLR0953 | 2662 |
| L1037 | Hypothetical protein SLR0953 | 2663 |
| L1038 | Hypothetical protein SLR0953 | 2664 |
| L1039 | Hypothetical protein SLR0953 | 2665 |
| L1040 | Hypothetical protein SLR0953 | 2666 |
| L1041 | Hypothetical protein YIGZ | 2667 |
| L1042 | Hypothetical protein YIGZ | 2668 |
| L1043 | Hypothetical protein YJIA | 2669 |
| L1044 | Hypothetical protein YJIA | 2670 |
| L1045 | Hypothetical protein YJIA | 2671 |
| L1046 | Hypothetical protein YJIA | 2672 |
| L1047 | Hypothetical protein YJIA | 2673 |
| L1048 | Hypothetical tRNA/rRNA methyltransferase YJFH | 2674 |
| L1049 | Hypothetical tRNA/rRNA methyltransferase YJFH | 2675 |
| L1050 | IcIR transcriptional regulator | 2676 |
| L1051 | IcIR transcriptional regulator | 2677 |
| L1052 | IcIR transcriptional regulator | 2678 |
| L1053 | IcIR transcriptional regulator | 2679 |
| L1054 | Integrase | 2680 |
| L1055 | Interferon, alpha-inducible protein (clone IFI-15k) | 2681 |
| L1056 | Interleukin-1 receptor, type I | AIF |
| L1057 | Interleukin-1 receptor, type I | 2682 |
| L1058 | Interleukin-1 receptor, type I | 2683 |
| L1059 | Interleukin-1 receptor, type I | 2684 |
| L1060 | Interleukin-12 subunit p40 | FFI |
| L1061 | Interleukin-12 subunit p40 | 2685 |
| L1062 | Interleukin-12 subunit p40 | 2686 |
| L1063 | Interleukin-12 subunit p40 | 2687 |
| L1064 | Interleukin-12 subunit p40 | 2688 |
| L1065 | Interleukin-12 subunit p40 | 2689 |
| L1066 | Interleukin-12 subunit p40 | 2690 |
| L1067 | Interleukin-12 subunit p40 | 2691 |
| L1068 | Interleukin-2 receptor alpha chain | 2692 |
| L1069 | Interleukin-2 receptor alpha chain | 2693 |
| L1070 | Internalin B | VTQ |
| L1071 | Internalin B | 2694 |
| L1072 | Internalin B | 2695 |
| L1073 | Internalin B | 2696 |
| L1074 | Internalin B | 2697 |
| L1075 | Internalin B | 2698 |
| L1076 | Internalin B | 2699 |
| L1077 | Internalin B | 2700 |
| L1078 | Internalin B | 2701 |
| L1079 | Internalin B | 2702 |
| L1080 | Internalin B | 2703 |
| L1081 | Internalin B | 2704 |
| L1082 | Internalin B | 2705 |
| L1083 | Intimin | SLV |
| L1084 | Intimin | 2706 |
| L1085 | Intimin | 2707 |
| L1086 | Intimin | 2708 |
| L1087 | Intron-encoded DNA endonuclease I-anil | 2709 |
| L1088 | Intron-encoded DNA endonuclease I-anil | 2710 |
| L1089 | Invasin | KST |
| L1090 | Invasin | 2711 |
| L1091 | Invasin | 2712 |
| L1092 | Invasin | 2713 |
| L1093 | Invasin | 2714 |
| L1094 | Invasin | 2715 |
| L1095 | Invasin | 2716 |
| L1096 | Invasin | 2717 |
| L1097 | Invasin | 2718 |
| L1098 | Invasin | 2719 |
| L1099 | Invasin | 2720 |
| L1100 | Invasin | 2721 |
| L1101 | Invasin | 2722 |
| L1102 | Iron hydrogenase 1 | GAE |
| L1103 | Iron hydrogenase 1 | 2723 |
| L1104 | Iron hydrogenase 1 | 2724 |
| L1105 | Iron hydrogenase 1 | 2725 |
| L1106 | Iron hydrogenase 1 | 2726 |
| L1107 | Iron hydrogenase 1 | 2727 |
| L1108 | Iron hydrogenase 1 | 2728 |
| L1109 | Iron hydrogenase 1 | 2729 |
| L1110 | Iron hydrogenase 1 | 2730 |
| L1111 | Iron hydrogenase 1 | 2731 |
| L1112 | Iron hydrogenase 1 | 2732 |
| L1113 | Iron hydrogenase 1 | 2733 |
| L1114 | Iron hydrogenase 1 | 2734 |
| L1115 | Iron hydrogenase 1 | 2735 |
| L1116 | Iron transport protein | 2736 |
| L1117 | Isoflavanone 4′-O-methyltransferase | 2737 |
| L1118 | Isoflavanone 4′-O-methyltransferase | 2738 |
| L1119 | Junctional adhesion molecule 1 | 2739 |
| L1120 | Junctional adhesion molecule 1 | 2740 |
| L1121 | Junctional adhesion molecule 1 | 2741 |
| L1122 | Kanamycin nucleotidyltransferase | 2742 |
| L1123 | Kanamycin nucleotidyltransferase | 2743 |
| L1124 | Kanamycin nucleotidyltransferase | 2744 |
| L1125 | Kanamycin nucleotidyltransferase | 2745 |
| L1126 | Kelch-like protein 11 | 2746 |
| L1127 | Kexin | ISE |
| L1128 | Kexin | 2747 |
| L1129 | Kexin | 2748 |
| L1130 | Kexin | 2749 |
| L1131 | Kexin | 2750 |
| L1132 | Kexin | 2751 |
| L1133 | Kexin | 2752 |
| L1134 | Kexin | 2753 |
| L1135 | Ku70 | 2754 |
| L1136 | Ku70 | 2755 |
| L1137 | Ku70 | 2756 |
| L1138 | Ku70 | 2757 |
| L1139 | Ku80 | 2758 |
| L1140 | Laccase-1 | 2759 |
| L1141 | Laccase-1 | 2760 |
| L1142 | Laccase-1 | 2761 |
| L1143 | Laccase-1 | 2762 |
| L1144 | Laminin | DKC |
| L1145 | L-aspartate dehydrogenase | SAS |
| L1146 | L-aspartate dehydrogenase | 2763 |
| L1147 | L-aspartate dehydrogenase | 2764 |
| L1148 | Leucine dehydrogenase | 2765 |
| L1149 | Leucine dehydrogenase | 2766 |
| L1150 | Light chain of HyHel10 antibody fragment (fab) | 2767 |
| L1151 | Lin2111 protein | 2768 |
| L1152 | Lin2111 protein | 2769 |
| L1153 | Lipopolysaccharide-responsive | 2770 |
| | and beige-like anchor protein | |
| L1154 | Lipopolysaccharide-responsive | 2771 |
| | and beige-like anchor protein | |
| L1155 | Lipovitellin (LV-1N, LV-1C) | 2772 |
| L1156 | Lipovitellin (LV-1N, LV-1C) | 2773 |
| L1157 | Lipovitellin (LV-1N, LV-1C) | 2774 |
| L1158 | Lipovitellin (LV-1N, LV-1C) | 2775 |
| L1159 | Lipovitellin (LV-1N, LV-1C) | 2776 |
| L1160 | Lipoxygenase-1 | 2777 |
| L1161 | Lipoxygenase-1 | 2778 |
| L1162 | Low affinity immunoglobulin | 2779 |
| | gamma Fc region receptor II-A | |
| L1163 | Luciferase | 2780 |
| L1164 | LysR-type regulatory protein | 2781 |
| L1165 | Macrolide-specific efflux protein MacA | ATE |
| L1166 | Macrolide-specific efflux protein MacA | 2782 |
| L1167 | Macrolide-specific efflux protein MacA | 2783 |
| L1168 | Magnesium transporter, putative | 2784 |
| L1169 | Main hemagglutinin component | 2785 |
| L1170 | Major centromere autoantigen B | 2786 |
| L1171 | Major surface antigen p30 | 2787 |
| L1172 | Major surface antigen p30 | 2788 |
| L1173 | Major vault protein | 2789 |
| L1174 | Major vault protein | 2790 |
| L1175 | Maltose phosphorylase | 2791 |
| L1176 | Maltose phosphorylase | 2792 |
| L1177 | Maltose phosphorylase | 2793 |
| L1178 | Maltose phosphorylase | 2794 |
| L1179 | Maltose phosphorylase | 2795 |
| L1180 | Manganese-dependent inorganic pyrophosphatase | 2796 |
| L1181 | Manganese-dependent inorganic pyrophosphatase | 2797 |
| L1182 | Mannan-binding lectin | 2798 |
| L1183 | Mannan-binding lectin | 2799 |
| L1184 | Mannan-binding lectin | 2800 |
| L1185 | Mannitol dehydrogenase | HNA |
| L1186 | Mannitol dehydrogenase | 2801 |
| L1187 | Membrane cofactor protein | RET |
| L1188 | Membrane cofactor protein | 2802 |
| L1189 | Membrane-associated prostaglandin E synthase-2 | 2803 |
| L1190 | Membrane-associated prostaglandin E synthase-2 | 2804 |
| L1191 | Membrane-associated prostaglandin E synthase-2 | 2805 |
| L1192 | Membrane-associated prostaglandin E synthase-2 | 2806 |
| L1193 | Membrane-associated prostaglandin E synthase-2 | 2807 |
| L1194 | Membrane-bound lytic murein transglycosylase A | 2808 |
| L1195 | Methionyl-tRNA synthetase | 2809 |
| L1196 | Methyl-accepting chemotaxis protein | VRP |
| L1197 | Methyl-accepting chemotaxis protein | 2810 |
| L1198 | Methyl-accepting chemotaxis protein | 2811 |
| L1199 | Methyl-accepting chemotaxis protein | 2812 |
| L1200 | Methyl-coenzyme M reductase | 2813 |
| L1201 | Methyl-coenzyme M reductase | 2814 |
| L1202 | Methyl-coenzyme M reductase | 2815 |
| L1203 | Methyl-coenzyme M reductase | 2816 |
| L1204 | Methylene tetrahydromethanopterin dehydrogenase | 2817 |
| L1205 | Methylene tetrahydromethanopterin dehydrogenase | 2818 |
| L1206 | Mg2+ transporter MgtE | 2819 |
| L1207 | Mg2+ transporter MgtE | 2820 |
| L1208 | Mg2+ transporter MgtE | 2821 |
| L1209 | Mitochondrial aconitase | 2822 |
| L1210 | Mitochondrial aconitase | 2823 |
| L1211 | Modification methylase TaqI | EGK |
| L1212 | Modification methylase TaqI | PAT |
| L1213 | Modification methylase TagI | 2824 |
| L1214 | Modification methylase TaqI | 2825 |
| L1215 | Modification methylase TaqI | 2826 |
| L1216 | Modification methylase TaqI | 2827 |
| L1217 | Modification methylase TagI | 2828 |
| L1218 | Modification methylase TagI | 2829 |
| L1219 | Modification methylase TaqI | 2830 |
| L1220 | Modification methylase TaqI | 2831 |
| L1221 | Multidrug-efflux transporter 1 regulator | 2832 |
| L1222 | Muramoyl-pentapeptide carboxypeptidase | 2833 |
| L1223 | MutL | 2834 |
| L1224 | MutL | 2835 |
| L1225 | MutL | 2836 |
| L1226 | MutL | 2837 |
| L1227 | MutL | 2838 |
| L1228 | MutL | 2839 |
| L1229 | MutL | 2840 |
| L1230 | MutL | 2841 |
| L1231 | MutL | 2842 |
| L1232 | MutM (Fpg) protein | 2843 |
| L1233 | MutM (Fpg) protein | 2844 |
| L1234 | MutM (Fpg) protein | 2845 |
| L1235 | MutM (Fpg) protein | 2846 |
| L1236 | Myotubularin-related protein 2 | THW |
| L1237 | Myotubularin-related protein 2 | 2847 |
| L1238 | Myotubularin-related protein 2 | 2848 |
| L1239 | Myotubularin-related protein 2 | 2849 |
| L1240 | Myotubularin-related protein 2 | 2850 |
| L1241 | Myotubularin-related protein 2 | 2851 |
| L1242 | N utilization substance protein A | EIP |
| L1243 | N utilization substance protein A | 2852 |
| L1244 | N utilization substance protein A | 2853 |
| L1245 | N utilization substance protein A | 2854 |
| L1246 | N-acetylglucosamine kinase | CAY |
| L1247 | N-acetylglucosamine kinase | ISP |
| L1248 | N-acetylglucosamine kinase | 2855 |
| L1249 | N-acyl-D-glutamate deacylase | 2856 |
| L1250 | N-acyl-D-glutamate deacylase | 2857 |
| L1251 | N-acyl-D-glutamate deacylase | 2858 |
| L1252 | N-acyl-D-glutamate deacylase | 2859 |
| L1253 | N-acyl-D-glutamate deacylase | 2860 |
| L1254 | N-acyl-D-glutamate deacylase | 2861 |
| L1255 | N-acyl-D-glutamate deacylase | 2862 |
| L1256 | NAD-dependent malic enzyme | 2863 |
| L1257 | NAD-dependent malic enzyme | 2864 |
| L1258 | NADH peroxidase | ADT |
| L1259 | NADH peroxidase | AVG |
| L1260 | NADH peroxidase | TLI |
| L1261 | NADH peroxidase | 2865 |
| L1262 | NADH peroxidase | 2866 |
| L1263 | NADH peroxidase | 2867 |
| L1264 | NADH peroxidase | 2868 |
| L1265 | NADH peroxidase | 2869 |
| L1266 | NADH peroxidase | 2870 |
| L1267 | NADH pyrophosphatase | 2871 |
| L1268 | Naphthalene 1,2-dioxygenase alpha subunit | 2872 |
| L1269 | Naphthalene 1,2-dioxygenase alpha subunit | 2873 |
| L1270 | NEDD8-activating enzyme E1 catalytic subunit | 2874 |
| L1271 | NEDD8-activating enzyme E1 regulatory subunit | 2875 |
| L1272 | NEDD8-activating enzyme E1 regulatory subunit | 2876 |
| L1273 | NEDD8-activating enzyme E1 regulatory subunit | 2877 |
| L1274 | Nei endonuclease VIII-Like 1 | 2878 |
| L1275 | Nei endonuclease VIII-Like 1 | 2879 |
| L1276 | Nei endonuclease VIII-Like 1 | 2880 |
| L1277 | Nei endonuclease VIII-Like 1 | 2881 |
| L1278 | Neural cell adhesion molecule 2 | 2882 |
| L1279 | Neural cell adhesion molecule 2 | 2883 |
| L1280 | Neural cell adhesion molecule 2 | 2884 |
| L1281 | Neural cell adhesion molecule 2 | 2885 |
| L1282 | Neural cell adhesion molecule 2 | 2886 |
| L1283 | Neuroplastin | 2887 |
| L1284 | Neuroplastin | 2888 |
| L1285 | Neuroplastin | 2889 |
| L1286 | Neutrophil cytosol factor 1 | 2890 |
| L1287 | Nickel responsive regulator | 2891 |
| L1288 | NifU-like protein 2, chloroplast | 2892 |
| L1289 | Nitric oxide reductase | ILM |
| L1290 | Nitric oxide reductase | 2893 |
| L1291 | Nitric oxide reductase | 2894 |
| L1292 | Nitric oxide reductase | 2895 |
| L1293 | Nitric oxide reductase | 2896 |
| L1294 | Nitric oxide reductase | 2897 |
| L1295 | NK receptor | 2898 |
| L1296 | Nuclear factor of activated t-cells, cytoplasmic2 | 2899 |
| L1297 | Nucleolin RBD 12 | 2900 |
| L1298 | O-GlcNAcase NagJ | 2901 |
| L1299 | Orange carotenoid protein | EGV |
| L1300 | Orange carotenoid protein | 2902 |
| L1301 | Orange carotenoid protein | 2903 |
| L1302 | Om/Lys/Arg decarboxylase family protein | LEL |
| L1303 | Orn/Lys/Arg decarboxylase family protein | 2904 |
| L1304 | Orn/Lys/Arg decarboxylase family protein | 2905 |
| L1305 | Om/Lys/Arg decarboxylase family protein | 2906 |
| L1306 | Om/Lys/Arg decarboxylase family protein | 2907 |
| L1307 | Om/Lys/Arg decarboxylase family protein | 2908 |
| L1308 | Orn/Lys/Arg decarboxylase family protein | 2909 |
| L1309 | Orn/Lys/Arg decarboxylase family protein | 2910 |
| L1310 | Osteoclast-stimulating factor 1 | 2911 |
| L1311 | Oxygen-independent coproporphyrinogen III oxidase | 2912 |
| L1312 | Oxygen-independent coproporphyrinogen III oxidase | 2913 |
| L1313 | Oxygen-independent coproporphyrinogen III oxidase | 2914 |
| L1314 | Oxygen-independent coproporphyrinogen III oxidase | 2915 |
| L1315 | Oxygen-independent coproporphyrinogen III oxidase | 2916 |
| L1316 | Oxygen-independent coproporphyrinogen III oxidase | 2917 |
| L1317 | Oxygen-independent coproporphyrinogen III oxidase | 2918 |
| L1318 | Oxygen-independent coproporphyrinogen III oxidase | 2919 |
| L1319 | Oxygen-independent coproporphyrinogen III oxidase | 2920 |
| L1320 | Oxygen-independent coproporphyrinogen III oxidase | 2921 |
| L1321 | Paraneoplastic encephalomyelitis antigen HuD | 2922 |
| L1322 | Paraneoplastic encephalomyelitis antigen HuD | 2923 |
| L1323 | Penicillin binding protein 4 | 2924 |
| L1324 | Penicillin binding protein 4 | 2925 |
| L1325 | Penicillin binding protein 4 | 2926 |
| L1326 | Penicillin binding protein 4 | 2927 |
| L1327 | Penicillin binding protein 4 | 2928 |
| L1328 | Penicillin binding protein 4 | 2929 |
| L1329 | Penicillin binding protein 4 | 2930 |
| L1330 | Peptide-N(4)-(N-acetyl-beta-D- | DGV |
| | glucosaminyl)asparagine amidase F | |
| L1331 | Peptide-N(4)-(N-acetyl-beta-D- | 2931 |
| | glucosaminyl)asparagine amidase F | |
| L1332 | Peptide-N(4)-(N-acetyl-beta-D- | 2932 |
| | glucosaminyl)asparagine amidase F | |
| L1333 | Peptide-N(4)-(N-acetyl-beta-D- | 2933 |
| | glucosaminyl)asparagine amidase F | |
| L1334 | Peroxisomal primary amine oxidase | 2934 |
| L1335 | Peroxisomal primary amine oxidase | 2935 |
| L1336 | Peroxisome biogenesis factor 1 | 2936 |
| L1337 | Pesticidial crystal protein Cry2Aa | 2937 |
| L1338 | Pesticidial crystal protein Cry2Aa | 2938 |
| L1339 | Pesticidial crystal protein Cry2Aa | 2939 |
| L1340 | Phase 1 flagellin | DLT |
| L1341 | Phase 1 flagellin | 2940 |
| L1342 | Phase 1 flagellin | 2941 |
| L1343 | Phase 1 flagellin | 2942 |
| L1344 | Phase 1 flagellin | 2943 |
| L1345 | Phase 1 flagellin | 2944 |
| L1346 | Phase 1 flagellin | 2945 |
| L1347 | Phase 1 flagellin | 2946 |
| L1348 | Phase 1 flagellin | 2947 |
| L1349 | Phase 1 flagellin | 2948 |
| L1350 | Phase 1 flagellin | 2949 |
| L1351 | Phase 1 flagellin | 2950 |
| L1352 | Phase 1 flagellin | 2951 |
| L1353 | Phenylalanyl-tRNA synthetase beta chain | LGL |
| L1354 | Phenylalanyl-tRNA synthetase beta chain | 2952 |
| L1355 | Phenylalanyl-tRNA synthetase beta chain | 2953 |
| L1356 | Phenylalanyl-tRNA synthetase beta chain | 2954 |
| L1357 | Phenylalanyl-tRNA synthetase beta chain | 2955 |
| L1358 | Phenylalanyl-tRNA synthetase beta chain | 2956 |
| L1359 | Phenylalanyl-tRNA synthetase beta chain | 2957 |
| L1360 | Phenylalanyl-tRNA synthetase beta chain | 2958 |
| L1361 | Phenylalanyl-tRNA synthetase beta chain | 2959 |
| L1362 | Phenylalanyl-tRNA synthetase beta chain | 2960 |
| L1363 | Phenylalanyl-tRNA synthetase beta chain | 2961 |
| L1364 | Phenylalanyl-tRNA synthetase beta chain | 2962 |
| L1365 | Phenylalanyl-tRNA synthetase beta chain | 2963 |
| L1366 | Phenylalanyl-tRNA synthetase beta chain | 2964 |
| L1367 | Phosphatase | 2965 |
| L1368 | Phosphatase | 2966 |
| L1369 | Phosphatase | 2967 |
| L1370 | Phosphatidylinositol transfer protein Sec14p | YGT |
| L1371 | Phosphatidylinositol transfer protein Sec14p | 2968 |
| L1372 | Phosphatidylinositol transfer protein Sec14p | 2969 |
| L1373 | Phosphatidylserine synthase | 2970 |
| L1374 | Phosphatidylserine synthase | 2971 |
| L1375 | Phosphatidylserine synthase | 2972 |
| L1376 | Phosphoglycolate phosphatase | 2973 |
| L1377 | Phosphoglycolate phosphatase | 2974 |
| L1378 | Phosphoglycolate phosphatase | 2975 |
| L1379 | Phosphoglycolate phosphatase | 2976 |
| L1380 | Phospholipase D | 2977 |
| L1381 | Phospholipase D | 2978 |
| L1382 | Phospholipase D | 2979 |
| L1383 | Phosphoribosylamine—glycine ligase | 2980 |
| L1384 | Phosphoribosylamine—glycine ligase | 2981 |
| L1385 | Phosphotransferase system, enzyme I | 2982 |
| L1386 | Photosystem II d1 protease | 2983 |
| L1387 | Photosystem II d1 protease | 2984 |
| L1388 | Photosystem II d1 protease | 2985 |
| L1389 | Photosystem II d1 protease | 2986 |
| L1390 | Photosystem II d1 protease | 2987 |
| L1391 | Phthalate dioxygenase reductase | 2988 |
| L1392 | P-hydroxybenzoate hydroxylase | DGL |
| L1393 | P-hydroxybenzoate hydroxylase | IDL |
| L1394 | P-hydroxybenzoate hydroxylase | RLK |
| L1395 | P-hydroxybenzoate hydroxylase | 2989 |
| L1396 | P-hydroxybenzoate hydroxylase | 2990 |
| L1397 | P-hydroxybenzoate hydroxylase | 2991 |
| L1398 | P-hydroxybenzoate hydroxylase | 2992 |
| L1399 | P-hydroxybenzoate hydroxylase | 2993 |
| L1400 | P-hydroxybenzoate hydroxylase | 2994 |
| L1401 | P-hydroxybenzoate hydroxylase | 2995 |
| L1402 | P-hydroxybenzoate hydroxylase | 2996 |
| L1403 | P-hydroxybenzoate hydroxylase | 2997 |
| L1404 | P-hydroxybenzoate hydroxylase | 2998 |
| L1405 | P-hydroxybenzoate hydroxylase | 2999 |
| L1406 | P-hydroxybenzoate hydroxylase | 3000 |
| L1407 | P-hydroxybenzoate hydroxylase | 3001 |
| L1408 | P-hydroxybenzoate hydroxylase | 3002 |
| L1409 | P-hydroxybenzoate hydroxylase | 3003 |
| L1410 | P-hydroxybenzoate hydroxylase | 3004 |
| L1411 | P-hydroxybenzoate hydroxylase | 3005 |
| L1412 | Phytase | LNF |
| L1413 | Phytase | QSN |
| L1414 | Phytase | 3006 |
| L1415 | Phytase | 3007 |
| L1416 | Phytase | 3008 |
| L1417 | Phytase | 3009 |
| L1418 | Phytase | 3010 |
| L1419 | Phytase | 3011 |
| L1420 | Phytase | 3012 |
| L1421 | Phytase | 3013 |
| L1422 | Pirin | LKS |
| L1423 | Pirin | SGE |
| L1424 | Pirin | 3014 |
| L1425 | Pirin | 3015 |
| L1426 | Pirin | 3016 |
| L1427 | Pirin | 3017 |
| L1428 | Pirin | 3018 |
| L1429 | Pirin | 3019 |
| L1430 | Poly(A) polymerase | 3020 |
| L1431 | Poly(A) polymerase | 3021 |
| L1432 | Poly(A) polymerase | 3022 |
| L1433 | Poly(A) polymerase | 3023 |
| L1434 | Poly(A) polymerase | 3024 |
| L1435 | Poly(A) polymerase | 3025 |
| L1436 | Poly(A) polymerase | 3026 |
| L1437 | Poly(A) polymerase | 3027 |
| L1438 | Poly(A) polymerase | 3028 |
| L1439 | Poly(A) polymerase | 3029 |
| L1440 | Poly(A) polymerase | 3030 |
| L1441 | Poly(A) polymerase | 3031 |
| L1442 | Poly(rC)-binding protein 2 | 3032 |
| L1443 | Polymerase x | 3033 |
| L1444 | Polymerase x | 3034 |
| L1445 | Polypeptide N-acetylgalactosaminyltransferase 2 | 3035 |
| L1446 | Polypeptide N-acetylgalactosaminyltransferase 2 | 3036 |
| L1447 | Polyphosphate kinase | 3037 |
| L1448 | Polyphosphate kinase | 3038 |
| L1449 | Polyphosphate kinase | 3039 |
| L1450 | Polypyrimidine tract-binding protein | 3040 |
| L1451 | Porcine pancreatic spasmolytic polypeptide | 3041 |
| L1452 | Possible 3-mercaptopyruvate sulfurtransferase | LFR |
| L1453 | Possible 3-mercaptopyruvate sulfurtransferase | YGM |
| L1454 | Possible 3-mercaptopyruvate sulfurtransferase | 3042 |
| L1455 | Possible 3-mercaptopyruvate sulfurtransferase | 3043 |
| L1456 | Possible 3-mercaptopyruvate sulfurtransferase | 3044 |
| L1457 | Postsynaptic density protein 95 | 3045 |
| L1458 | Postsynaptic density protein 95 | 3046 |
| L1459 | Predicted sugar phosphatases of the HAD superfamily | IAI |
| L1460 | Predicted sugar phosphatases of the HAD superfamily | 3047 |
| L1461 | Predicted sugar phosphatases of the HAD superfamily | 3048 |
| L1462 | Predicted sugar phosphatases of the HAD superfamily | 3049 |
| L1463 | Predicted sugar phosphatases of the HAD superfamily | 3050 |
| L1464 | Predicted sugar phosphatases of the HAD superfamily | 3051 |
| L1465 | Predicted sugar phosphatases of the HAD superfamily | 3052 |
| L1466 | Predicted sugar phosphatases of the HAD superfamily | 3053 |
| L1467 | Predicted sugar phosphatases of the HAD superfamily | 3054 |
| L1468 | Preprotein translocase SecA | ITF |
| L1469 | Preprotein translocase SecA | LID |
| L1470 | Preprotein translocase SecA | 3055 |
| L1471 | Preprotein translocase SecA | 3056 |
| L1472 | Preprotein translocase SecA | 3057 |
| L1473 | Preprotein translocase SecA | 3058 |
| L1474 | Preprotein translocase SecA | 3059 |
| L1475 | Preprotein translocase SecA | 3060 |
| L1476 | Preprotein translocase SecA | 3061 |
| L1477 | Preprotein translocase SecA | 3062 |
| L1478 | Preprotein translocase SecA | 3063 |
| L1479 | Preprotein translocase SecA | 3064 |
| L1480 | Preprotein translocase SecA | 3065 |
| L1481 | Preprotein translocase SecA | 3066 |
| L1482 | Preprotein translocase SecA | 3067 |
| L1483 | Preprotein translocase SecA | 3068 |
| L1484 | Preprotein translocase SecA | 3069 |
| L1485 | Preprotein translocase SecA | 3070 |
| L1486 | Preprotein translocase SecA | 3071 |
| L1487 | PrfA | ING |
| L1488 | Probable 16s rRNA-processing protein RimM | 3072 |
| L1489 | Probable biphenyl-2,3-diol 1,2-dioxygenase BphC | 3073 |
| L1490 | Probable chorismate mutase | LLA |
| L1491 | Probable chorismate mutase | 3074 |
| L1492 | Probable chorismate mutase | 3075 |
| L1493 | Probable ferredoxin-dependent nitrite reductase NirA | VPL |
| L1494 | Probable ferredoxin-dependent nitrite reductase NirA | WGI |
| L1495 | Probable ferredoxin-dependent nitrite reductase NirA | 3076 |
| L1496 | Probable ferredoxin-dependent nitrite reductase NirA | 3077 |
| L1497 | Probable ferredoxin-dependent nitrite reductase NirA | 3078 |
| L1498 | Probable ferredoxin-dependent nitrite reductase NirA | 3079 |
| L1499 | Probable ferredoxin-dependent nitrite reductase NirA | 3080 |
| L1500 | Probable ferredoxin-dependent nitrite reductase NirA | 3081 |
| L1501 | Probable ferredoxin-dependent nitrite reductase NirA | 3082 |
| L1502 | Probable ferredoxin-dependent nitrite reductase NirA | 3083 |
| L1503 | Probable ferredoxin-dependent nitrite reductase NirA | 3084 |
| L1504 | Probable ferredoxin-dependent nitrite reductase NirA | 3085 |
| L1505 | Probable ferredoxin-dependent nitrite reductase NirA | 3086 |
| L1506 | Probable ferredoxin-dependent nitrite reductase NirA | 3087 |
| L1507 | Probable galactokinase | 3088 |
| L1508 | Probable galactokinase | 3089 |
| L1509 | Probable galactokinase | 3090 |
| L1510 | Probable galactokinase | 3091 |
| L1511 | Probable galactokinase | 3092 |
| L1512 | Probable galactokinase | 3093 |
| L1513 | Probable galactokinase | 3094 |
| L1514 | Probable galactokinase | 3095 |
| L1515 | Probable galactokinase | 3096 |
| L1516 | Probable galactokinase | 3097 |
| L1517 | Probable galactokinase | 3098 |
| L1518 | Probable galactokinase | 3099 |
| L1519 | Probable glutathione S-transferase | 3100 |
| L1520 | Probable GST-related protein | 3101 |
| L1521 | Probable HPr(Ser) kinase/phosphatase | 3102 |
| L1522 | Probable thiosulfate sulfur transferase | 3103 |
| L1523 | Probable thiosulfate sulfur transferase | 3104 |
| L1524 | Probable thiosulfate sulfur transferase | 3105 |
| L1525 | Probable thiosulfate sulfur transferase | 3106 |
| L1526 | Probable thiosulfate sulfur transferase | 3107 |
| L1527 | Probable thiosulfate sulfur transferase | 3108 |
| L1528 | Probable thiosulfate sulfur transferase | 3109 |
| L1529 | Probable thiosulfate sulfur transferase | 3110 |
| L1530 | Probable tRNA pseudouridine synthase D | 3111 |
| L1531 | Probable tRNA pseudouridine synthase D | 3112 |
| L1532 | Probable tRNA pseudouridine synthase D | 3113 |
| L1533 | Probable tRNA pseudouridine synthase D | 3114 |
| L1534 | Probable tRNA pseudouridine synthase D | 3115 |
| L1535 | Probable tRNA pseudouridine synthase D | 3116 |
| L1536 | Programed cell death protein 8 | SKE |
| L1537 | Programed cell death protein 8 | TLQ |
| L1538 | Programed cell death protein 8 | 3117 |
| L1539 | Programed cell death protein 8 | 3118 |
| L1540 | Programed cell death protein 8 | 3119 |
| L1541 | Programed cell death protein 8 | 3120 |
| L1542 | Programed cell death protein 8 | 3121 |
| L1543 | Programed cell death protein 8 | 3122 |
| L1544 | Programed cell death protein 8 | 3123 |
| L1545 | Programed cell death protein 8 | 3124 |
| L1546 | Programed cell death protein 8 | 3125 |
| L1547 | Programed cell death protein 8 | 3126 |
| L1548 | Programed cell death protein 8 | 3127 |
| L1549 | Programed cell death protein 8 | 3128 |
| L1550 | Programed cell death protein 8 | 3129 |
| L1551 | Programed cell death protein 8 | 3130 |
| L1552 | Programed cell death protein 8 | 3131 |
| L1553 | Programed cell death protein 8 | 3132 |
| L1554 | Programed cell death protein 8 | 3133 |
| L1555 | Programed cell death protein 8 | 3134 |
| L1556 | Proline oxidase | 3135 |
| L1557 | Prolyl-tRNA synthetase | 3136 |
| L1558 | Prostaglandin G/H synthase 1 | PEI |
| L1559 | Prostaglandin G/H synthase 1 | 3137 |
| L1560 | Protease | 3138 |
| L1561 | Protease | 3139 |
| L1562 | Protease | 3140 |
| L1563 | Protease DegS | 3141 |
| L1564 | Protease DegS | 3142 |
| L1565 | Protease DegS | 3143 |
| L1566 | Protease DegS | 3144 |
| L1567 | Protease III | NAR |
| L1568 | Protease III | RNP |
| L1569 | Protease III | 3145 |
| L1570 | Protease III | 3146 |
| L1571 | Protease III | 3147 |
| L1572 | Protease III | 3148 |
| L1573 | Protease III | 3149 |
| L1574 | Protease III | 3150 |
| L1575 | Protease III | 3151 |
| L1576 | Protease III | 3152 |
| L1577 | Protease III | 3153 |
| L1578 | Protease III | 3154 |
| L1579 | Protease III | 3155 |
| L1580 | Protease III | 3156 |
| L1581 | Protease III | 3157 |
| L1582 | Protease III | 3158 |
| L1583 | Protease III | 3159 |
| L1584 | Protease III | 3160 |
| L1585 | Protease III | 3161 |
| L1586 | Protease III | 3162 |
| L1587 | Protease III | 3163 |
| L1588 | Protease III | 3164 |
| L1589 | Protection of telomeres 1 | 3165 |
| L1590 | Protection of telomeres 1 | 3166 |
| L1591 | Protein (CD58) | 3167 |
| L1592 | Protein (CRP1) | 3168 |
| L1593 | Protein (DNA polymerase) | 3169 |
| L1594 | Protein (DNA polymerase) | 3170 |
| L1595 | Protein (DNA polymerase) | 3171 |
| L1596 | Protein (electron transfer flavoprotein) | 3172 |
| L1597 | Protein (electron transfer flavoprotein) | 3173 |
| L1598 | Protein (Ffh) | 3174 |
| L1599 | Protein (Ffh) | 3175 |
| L1600 | Protein (Ffh) | 3176 |
| L1601 | Protein (Ffh) | 3177 |
| L1602 | Protein (Ffh) | 3178 |
| L1603 | Protein (FokI restriction endonuclease) | 3179 |
| L1604 | Protein (FokI restriction endonuclease) | 3180 |
| L1605 | Protein (FokI restriction endonuclease) | 3181 |
| L1606 | Protein (FokI restriction endonuclease) | 3182 |
| L1607 | Protein (FokI restriction endonuclease) | 3183 |
| L1608 | Protein (FokI restriction endonuclease) | 3184 |
| L1609 | Protein (FokI restriction endonuclease) | 3185 |
| L1610 | Protein (FokI restriction endonuclease) | 3186 |
| L1611 | Protein (FokI restriction endonuclease) | 3187 |
| L1612 | Protein (neural cell adhesion molecule) | 3188 |
| L1613 | Protein (neural cell adhesion molecule) | 3189 |
| L1614 | Protein (neural cell adhesion molecule) | 3190 |
| L1615 | Protein (nine-haem cytochrome c) | FTH |
| L1616 | Protein (nine-haem cytochrome c) | 3191 |
| L1617 | Protein (nine-haem cytochrome c) | 3192 |
| L1618 | Protein (nine-haem cytochrome c) | 3193 |
| L1619 | Protein (nine-haem cytochrome c) | 3194 |
| L1620 | Protein (nine-haem cytochrome c) | 3195 |
| L1621 | Protein (nine-haem cytochrome c) | 3196 |
| L1622 | Protein (nine-haem cytochrome c) | 3197 |
| L1623 | Protein (nine-haem cytochrome c) | 3198 |
| L1624 | Protein (protease/helicase NS3) | 3199 |
| L1625 | Protein (protease/helicase NS3) | 3200 |
| L1626 | Protein (protease/helicase NS3) | 3201 |
| L1627 | Protein (protease/helicase NS3) | 3202 |
| L1628 | Protein disulfide oxidoreductase | 3203 |
| L1629 | Protein disulfide oxidoreductase | 3204 |
| L1630 | Protein disulfide-isomerase A4 | 3205 |
| L1631 | Protein kinase PKR | 3206 |
| L1632 | Protein kinase PKR | 3207 |
| L1633 | Protein TolB | VNK |
| L1634 | Protein TolB | 3208 |
| L1635 | Protein TolB | 3209 |
| L1636 | Protein TolB | 3210 |
| L1637 | Protein TolB | 3211 |
| L1638 | Protein TolB | 3212 |
| L1639 | Protein TolB | 3213 |
| L1640 | Protein translation elongation factor 1A | 3214 |
| L1641 | Protein transport protein Sec24 | DRN |
| L1642 | Protein transport protein Sec24 | 3215 |
| L1643 | Protein transport protein Sec24 | 3216 |
| L1644 | Protein transport protein Sec24 | 3217 |
| L1645 | Protein transport protein Sec24 | 3218 |
| L1646 | Protein transport protein Sec24 | 3219 |
| L1647 | Protein transport protein Sec24 | 3220 |
| L1648 | Protein transport protein Sec24 | 3221 |
| L1649 | Protein transport protein Sec24 | 3222 |
| L1650 | Pseudouridine synthase CBF5 | AIQ |
| L1651 | Pseudouridine synthase CBF5 | 3223 |
| L1652 | Pseudouridine synthase CBF5 | 3224 |
| L1653 | Putative acetylglutamate synthase | 3225 |
| L1654 | Putative acetylglutamate synthase | 3226 |
| L1655 | Putative acetylglutamate synthase | 3227 |
| L1656 | Putative family 31 glucosidase Yicl | 3228 |
| L1657 | Putative family 31 glucosidase Yicl | 3229 |
| L1658 | Putative family 31 glucosidase Yicl | 3230 |
| L1659 | Putative glutathione transferase | 3231 |
| L1660 | Putative glutathione transferase | 3232 |
| L1661 | Putative glutathione transferase | 3233 |
| L1662 | Putative GNTR-family transcriptional regulator | 3234 |
| L1663 | Putative GNTR-family transcriptional regulator | 3235 |
| L1664 | Putative GNTR-family transcriptional regulator | 3236 |
| L1665 | Putative HTH-type transcriptional regulator PH0061 | 3237 |
| L1666 | Putative HTH-type transcriptional regulator PH1519 | 3238 |
| L1667 | Putative HTH-type transcriptional regulator PH1519 | 3239 |
| L1668 | Putative metallopeptidase | 3240 |
| L1669 | Putative N-acetylmannosamine kinase | 3241 |
| L1670 | Putative N-acetylmannosamine kinase | 3242 |
| L1671 | Putative N-acetylmannosamine kinase | 3243 |
| L1672 | Putative NADP oxidoreductase BF3122 | 3244 |
| L1673 | Putative NADP oxidoreductase BF3122 | 3245 |
| L1674 | Putative NADP oxidoreductase BF3122 | 3246 |
| L1675 | Putative NADP oxidoreductase BF3122 | 3247 |
| L1676 | Putative oxidoreductase | 3248 |
| L1677 | Putative secreted alpha-galactosidase | PLP |
| L1678 | Putative secreted alpha-galactosidase | TNG |
| L1679 | Putative secreted alpha-galactosidase | 3249 |
| L1680 | Putative secreted alpha-galactosidase | 3250 |
| L1681 | Putative secreted alpha-galactosidase | 3251 |
| L1682 | Putative tagatose-6-phosphate ketose/aldose isomerase | DKA |
| L1683 | Putative tagatose-6-phosphate ketose/aldose isomerase | 3252 |
| L1684 | Putative tagatose-6-phosphate ketose/aldose isomerase | 3253 |
| L1685 | Putative tagatose-6-phosphate ketose/aldose isomerase | 3254 |
| L1686 | Putative transcriptional regulator GntR | 3255 |
| L1687 | Putative transcriptional repressor (TetR/AcrR family) | KFR |
| L1688 | Putative transcriptional repressor (TetR/AcrR family) | 3256 |
| L1689 | Putative uncharacterized protein | 3257 |
| L1690 | Putative uncharacterized protein | 3258 |
| L1691 | Putative uncharacterized protein | 3259 |
| L1692 | Putative uncharacterized protein | 3260 |
| L1693 | Putative uncharacterized protein | 3261 |
| L1694 | Putative uncharacterized protein | 3262 |
| L1695 | Putative uncharacterized protein | 3263 |
| L1696 | Putative uncharacterized protein | 3264 |
| L1697 | Putative uncharacterized protein | 3265 |
| L1698 | Pyruvate decarboxylase | CAA |
| L1699 | Pyruvate decarboxylase | 3266 |
| L1700 | Pyruvate decarboxylase | 3267 |
| L1701 | Pyruvate decarboxylase | 3268 |
| L1702 | Pyruvate decarboxylase | 3269 |
| L1703 | Pyruvate decarboxylase | 3270 |
| L1704 | Pyruvate dehydrogenase [lipoamide] | YVP |
| | kinase isozyme 2, mitochondrial | |
| L1705 | Pyruvate dehydrogenase [lipoamide] | 3271 |
| | kinase isozyme 2, mitochondrial | |
| L1706 | Pyruvate dehydrogenase [lipoamide] | 3272 |
| | kinase isozyme 2, mitochondrial | |
| L1707 | Pyruvate dehydrogenase E1 | 3273 |
| | component subunit beta, mitochondrial | |
| L1708 | Pyruvate dehydrogenase E1 | 3274 |
| | component subunit beta, mitochondrial | |
| L1709 | Pyruvate dehydrogenase E1 | 3275 |
| | component subunit beta, mitochondrial | |
| L1710 | Pyruvate phosphate dikinase | FNP |
| L1711 | Pyruvate phosphate dikinase | SAL |
| L1712 | Pyruvate phosphate dikinase | 3276 |
| L1713 | Pyruvate phosphate dikinase | 3277 |
| L1714 | Pyruvate phosphate dikinase | 3278 |
| L1715 | Pyruvate phosphate dikinase | 3279 |
| L1716 | Pyruvate phosphate dikinase | 3280 |
| L1717 | Pyruvate phosphate dikinase | 3281 |
| L1718 | Pyruvate phosphate dikinase | 3282 |
| L1719 | Pyruvate phosphate dikinase | 3283 |
| L1720 | Pyruvate phosphate dikinase | 3284 |
| L1721 | Pyruvate phosphate dikinase | 3285 |
| L1722 | Pyruvate-ferredoxin oxidoreductase | VRL |
| L1723 | Pyruvate-ferredoxin oxidoreductase | 3286 |
| L1724 | Pyruvate-ferredoxin oxidoreductase | 3287 |
| L1725 | Pyruvate-ferredoxin oxidoreductase | 3288 |
| L1726 | Pyruvate-ferredoxin oxidoreductase | 3289 |
| L1727 | Pyruvate-ferredoxin oxidoreductase | 3290 |
| L1728 | Pyruvate-ferredoxin oxidoreductase | 3291 |
| L1729 | Pyruvate-ferredoxin oxidoreductase | 3292 |
| L1730 | Pyruvate-ferredoxin oxidoreductase | 3293 |
| L1731 | Pyruvate-ferredoxin oxidoreductase | 3294 |
| L1732 | Pyruvate-ferredoxin oxidoreductase | 3295 |
| L1733 | Pyruvate-ferredoxin oxidoreductase | 3296 |
| L1734 | Pyruvate-ferredoxin oxidoreductase | 3297 |
| L1735 | Pyruvate-ferredoxin oxidoreductase | 3298 |
| L1736 | Pyruvate-ferredoxin oxidoreductase | 3299 |
| L1737 | Pyruvate-ferredoxin oxidoreductase | 3300 |
| L1738 | Pyruvate-ferredoxin oxidoreductase | 3301 |
| L1739 | Pyruvate-ferredoxin oxidoreductase | 3302 |
| L1740 | Pyruvate-ferredoxin oxidoreductase | 3303 |
| L1741 | Pyruvate-ferredoxin oxidoreductase | 3304 |
| L1742 | Quinohemoprotein amine dehydrogenase 60 kDa subunit | 3305 |
| L1743 | Quinohemoprotein amine dehydrogenase 60 kDa subunit | 3306 |
| L1744 | Quinohemoprotein amine dehydrogenase 60 kDa subunit | 330 |
| L1745 | Quinohemoprotein amine dehydrogenase 60 kDa subunit | 3308 |
| L1746 | Quinohemoprotein amine dehydrogenase 60 kDa subunit | 3309 |
| L1747 | Quinohemoprotein amine dehydrogenase 60 kDa subunit | 3310 |
| L1748 | Quinohemoprotein amine dehydrogenase 60 kDa subunit | 3311 |
| L1749 | Quinohemoprotein amine dehydrogenase 60 kDa subunit | 3312 |
| L1750 | Quinohemoprotein amine dehydrogenase 60 kDa subunit | 3313 |
| L1751 | Quinohemoprotein amine dehydrogenase 60 kDa subunit | 3314 |
| L1752 | Rag1 | 3315 |
| L1753 | Rag1 | 3316 |
| L1754 | Receptor-type tyrosine-protein phosphatase Mu | 3317 |
| L1755 | Receptor-type tyrosine-protein phosphatase Mu | 3318 |
| L1756 | RecG | 3319 |
| L1757 | RecG | 3320 |
| L1758 | RecG | 3321 |
| L1759 | RecG | 3322 |
| L1760 | RecG | 3323 |
| L1761 | RecG | 3324 |
| L1762 | RecG | 3325 |
| L1763 | RecG | 3326 |
| L1764 | RecG | 3327 |
| L1765 | RecG | 3328 |
| L1766 | RecG | 3329 |
| L1767 | RecG | 3330 |
| L1768 | Recombination endonuclease VII | 3331 |
| L1769 | Recombining binding protein suppressor of hairless | 3332 |
| L1770 | Restriction endonuclease | ERV |
| L1771 | Restriction endonuclease | 3333 |
| L1772 | Restriction endonuclease | 3334 |
| L1773 | Restriction endonuclease | 3335 |
| L1774 | Retinaldehyde-binding protein 1 | QYP |
| L1775 | Retinaldehyde-binding protein 1 | 3336 |
| L1776 | Retinaldehyde-binding protein 1 | 3337 |
| L1777 | Retinoblastoma pocket | 3338 |
| L1778 | RfcS | ITD |
| L1779 | RfcS | LTE |
| L1780 | RfcS | 3339 |
| L1781 | RfcS | 3340 |
| L1782 | RfcS | 3341 |
| L1783 | RfcS | 3342 |
| L1784 | RfcS | 3343 |
| L1785 | Rhamnogalacturonase B | 3344 |
| L1786 | Rhamnogalacturonase B | 3345 |
| L1787 | Rhamnogalacturonase B | 3346 |
| L1788 | Rhamnogalacturonase B | 3347 |
| L1789 | Rhamnogalacturonase B | 3348 |
| L1790 | Rhodniin | 3349 |
| L1791 | Rhodniin | 3350 |
| L1792 | Riboflavin synthase | 3351 |
| L1793 | Ribonuclease D | 3352 |
| L1794 | Ribonuclease D | 3353 |
| L1795 | Ribonuclease D | 3354 |
| L1796 | Ribonuclease TTHA0252 | 3355 |
| L1797 | Ribonuclease TTHA0252 | 3356 |
| L1798 | Ribonuclease TTHA0252 | 3357 |
| L1799 | Ribonuclease TTHA0252 | 3358 |
| L1800 | Ribonuclease TTHA0252 | 3359 |
| L1801 | Ribonuclease TTHA0252 | 3360 |
| L1802 | Ribonucleotide reductase r1 protein | 3361 |
| L1803 | Ribonucleotide reductase r1 protein | 3362 |
| L1804 | Ribonucleotide reductase r1 protein | 3363 |
| L1805 | Ribonucleotide reductase r1 protein | 3364 |
| L1806 | Ribonucleotide reductase r1 protein | 3365 |
| L1807 | Ribonucleotide reductase r1 protein | 3366 |
| L1808 | Ribosome maturation factor RimM | 3367 |
| L1809 | Ribulose-1,5 bisphosphate | RHA |
| | carboxylase/oxygenase large subunit N- | |
| | methyltransferase | |
| L1810 | Ribulose-1,5 bisphosphate | 3368 |
| | carboxylase/oxygenase large subunit N- | |
| | methyltransferase | |
| L1811 | Rigid extended P-rich | 3369 |
| L1812 | Rigid extended P-rich | 3370 |
| L1813 | Rigid extended P-rich | 3371 |
| L1814 | Rigid extended P-rich | 3372 |
| L1815 | Rigid extended P-rich | 3373 |
| L1816 | Rigid extended P-rich | 3374 |
| L1817 | Rigid extended P-rich | 3375 |
| L1818 | Rigid extended P-rich | 3376 |
| L1819 | Rigid extended P-rich | 3377 |
| L1820 | Rigid extended P-rich | 3378 |
| L1821 | Rigid extended P-rich | 3379 |
| L1822 | Rigid extended P-rich | 3380 |
| L1823 | Rigid extended P-rich | 3381 |
| L1824 | Rigid extended P-rich | 3382 |
| L1825 | Rigid extended P-rich | 3383 |
| L1826 | Rigid helical | 3384 |
| L1827 | Rigid helical | 3385 |
| L1828 | Rigid helical | 3386 |
| L1829 | Rigid helical | 3387 |
| L1830 | Rigid helical | 3388 |
| L1831 | Rigid helical | 3389 |
| L1832 | Rigid helical | 3390 |
| L1833 | Rigid helical | 3391 |
| L1834 | RNA binding domain of rho | 3392 |
| | transcription termination factor | |
| L1835 | RNA binding protein ZFa | 3393 |
| L1836 | Rob transcription factor | 3394 |
| L1837 | Rob transcription factor | 3395 |
| L1838 | RP2 lipase | 3396 |
| L1839 | Rubrerythrin | 3397 |
| L1840 | S-adenosylmethionine synthetase | 3398 |
| L1841 | Sarcoplasmic/endoplasmic reticulum calcium ATPase 1 | QFD |
| L1842 | Sarcoplasmic/endoplasmic reticulum calcium ATPase 1 | 3399 |
| L1843 | Sarcoplasmic/endoplasmic reticulum calcium ATPase 1 | 3400 |
| L1844 | Sarcoplasmic/endoplasmic reticulum calcium ATPase 1 | 3401 |
| L1845 | Sarcoplasmic/endoplasmic reticulum calcium ATPase 1 | 3402 |
| L1846 | Sarcoplasmic/endoplasmic reticulum calcium ATPase 1 | 3403 |
| L1847 | Sarcoplasmic/endoplasmic reticulum calcium ATPase 1 | 3404 |
| L1848 | Sarcoplasmic/endoplasmic reticulum calcium ATPase 1 | 3405 |
| L1849 | Sarcoplasmic/endoplasmic reticulum calcium ATPase 1 | 3406 |
| L1850 | Sarcoplasmic/endoplasmic reticulum calcium ATPase 1 | 3407 |
| L1851 | Sarcoplasmic/endoplasmic reticulum calcium ATPase 1 | 3408 |
| L1852 | Sarcoplasmic/endoplasmic reticulum calcium ATPase 1 | 3409 |
| L1853 | Sarcoplasmic/endoplasmic reticulum calcium ATPase 1 | 3410 |
| L1854 | Sarcoplasmic/endoplasmic reticulum calcium ATPase 1 | 3411 |
| L1855 | Sarcoplasmic/endoplasmic reticulum calcium ATPase 1 | 3412 |
| L1856 | Sarcoplasmic/endoplasmic reticulum calcium ATPase 1 | 3413 |
| L1857 | Sarcoplasmic/endoplasmic reticulum calcium ATPase 1 | 3414 |
| L1858 | Sarcoplasmic/endoplasmic reticulum calcium ATPase 1 | 3415 |
| L1859 | Sarcoplasmic/endoplasmic reticulum calcium ATPase 1 | 3416 |
| L1860 | Sarcoplasmic/endoplasmic reticulum calcium ATPase 1 | 3417 |
| L1861 | Sarcoplasmic/endoplasmic reticulum calcium ATPase 1 | 3418 |
| L1862 | Sarcoplasmic/endoplasmic reticulum calcium ATPase 1 | 3419 |
| L1863 | Sarcoplasmic/endoplasmic reticulum calcium ATPase 1 | 3420 |
| L1864 | Sarcoplasmic/endoplasmic reticulum calcium ATPase 1 | 3421 |
| L1865 | Sarcoplasmic/endoplasmic reticulum calcium ATPase 1 | 3422 |
| L1866 | Sarcoplasmic/endoplasmic reticulum calcium ATPase 1 | 3423 |
| L1867 | Scavenger mRNA-decapping enzyme DcpS | ETG |
| L1868 | Scavenger mRNA-decapping enzyme DcpS | NIT |
| L1869 | Scavenger mRNA-decapping enzyme DcpS | 3424 |
| L1870 | Scavenger mRNA-decapping enzyme DcpS | 3425 |
| L1871 | Sec18p (residues 22-210) | 3426 |
| L1872 | Sec18p (residues 22-210) | 3427 |
| L1873 | Sensor protein | 3428 |
| L1874 | Sensor protein | 3429 |
| L1875 | Septum site-determining protein MinC | 3430 |
| L1876 | Serine acetyltransferase | 3431 |
| L1877 | Serine protease/NTPase/helicase NS3 | 3432 |
| L1878 | Serine protease/NTPase/helicase NS3 | 3433 |
| L1879 | Serine protease/NTPase/helicase NS3 | 3434 |
| L1880 | Serine rich linker | 3435 |
| L1881 | Serine rich linker | 3436 |
| L1882 | Serine rich linker | 3437 |
| L1883 | Serine rich linker | 3438 |
| L1884 | Serine rich linker | 3439 |
| L1885 | Serine rich linker | 3440 |
| L1886 | Serine rich linker | 3441 |
| L1887 | Seryl-tRNA synthetase | 3442 |
| L1888 | Sialidase | 3443 |
| L1889 | Sialidase B | SLT |
| L1890 | Sialidase B | VRE |
| L1891 | Sialidase B | 3444 |
| L1892 | Sialidase B | 3445 |
| L1893 | Sialidase B | 3446 |
| L1894 | Sialidase B | 3447 |
| L1895 | Sialidase B | 3448 |
| L1896 | Sialidase B | 3449 |
| L1897 | SIgnal peptIdase I | SRR |
| L1898 | SIgnal peptIdase I | 3450 |
| L1899 | SIgnal peptIdase I | 3451 |
| L1900 | SIgnal peptIdase I | 3452 |
| L1901 | SIgnal peptIdase I | 3453 |
| L1902 | SIgnal peptIdase I | 3454 |
| L1903 | SIgnal peptIdase I | 3455 |
| L1904 | SIgnal peptIdase I | 3456 |
| L1905 | SIgnal peptIdase I | 3457 |
| L1906 | SIgnal peptIdase I | 3458 |
| L1907 | SIgnal peptIdase I | 3459 |
| L1908 | Signal recognition particle protein | 3460 |
| L1909 | Signal transducer and activator | NDE |
| | of transcription1-alpha/beta | |
| L1910 | Signal transducer and activator | SSF |
| | of transcription1-alpha/beta | |
| L1911 | Signal transducer and activator | 3461 |
| | of transcription1-alpha/beta | |
| L1912 | Signal transducer and activator | 3462 |
| | of transcription1-alpha/beta | |
| L1913 | Signal transducer and activator | 3463 |
| | of transcription1-alpha/beta | |
| L1914 | Signal transducer and activator | 3464 |
| | of transcription1-alpha/beta | |
| L1915 | Signal transduction protein CBL | 3465 |
| L1916 | Signal transduction protein CBL | 3466 |
| L1917 | Similar to RAD54-like | AKP |
| L1918 | Similar to RAD54-like | EYF |
| L1919 | Similar to RAD54-like | RFE |
| L1920 | Similar to RAD54-like | 3467 |
| L1921 | Similar to RAD54-like | 3468 |
| L1922 | Similar to RAD54-like | 3469 |
| L1923 | Similar to RAD54-like | 3470 |
| L1924 | Similar to RAD54-like | 3471 |
| L1925 | Similar to RAD54-like | 3472 |
| L1926 | Similar to RAD54-like | 3473 |
| L1927 | Similar to RAD54-like | 3474 |
| L1928 | Similar to RAD54-like | 3475 |
| L1929 | Similar to RAD54-like | 3476 |
| L1930 | SKD1 protein | LMQ |
| L1931 | SKD1 protein | 3477 |
| L1932 | SKD1 protein | 3478 |
| L1933 | SKDI protein | 3479 |
| L1934 | SKD1 protein | 3480 |
| L1935 | SKD1 protein | 3481 |
| L1936 | Sll1358 protein | 3482 |
| L1937 | Sll1358 protein | 3483 |
| L1938 | Sll1358 protein | 3484 |
| L1939 | Sll1358 protein | 3485 |
| L1940 | Soluble IFN alpha/beta receptor | 3486 |
| L1941 | Soluble IFN alpha/beta receptor | 3487 |
| L1942 | Sporozoite-specific SAG protein | 3488 |
| L1943 | Staphylococcal accessory regulator a homologue | 3489 |
| L1944 | Staphylococcal nuclease domain-containing protein 1 | 3490 |
| L1945 | Staphylococcal nuclease domain-containing protein 1 | 3491 |
| L1946 | Staphylococcal nuclease domain-containing protein 1 | 3492 |
| L1947 | Staphylococcal nuclease domain-containing protein 1 | 3493 |
| L1948 | Staphylococcal nuclease domain-containing protein 1 | 3494 |
| L1949 | Staphylococcal nuclease domain-containing protein 1 | 3495 |
| L1950 | Stat protein | 3496 |
| L1951 | Stat protein | 3497 |
| L1952 | Stat protein | 3498 |
| L1953 | Stat protein | 3499 |
| L1954 | Stat protein | 3500 |
| L1955 | Stat protein | 3501 |
| L1956 | Stat protein | 3502 |
| L1957 | Stat protein | 3503 |
| L1958 | Stat protein | 3504 |
| L1959 | Stat protein | 3505 |
| L1960 | Stat protein | 3506 |
| L1961 | Stat protein | 3507 |
| L1962 | Stat protein | 3508 |
| L1963 | Stat protein | 3509 |
| L1964 | Stat protein | 3510 |
| L1965 | Subtilisin-like protease | 3511 |
| L1966 | Succinyl-CoA ligase [GDP-forming] | 3512 |
| | alpha-chain, mitochondrial | |
| L1967 | Succinyl-CoA ligase [GDP-forming] | 3513 |
| | alpha-chain, mitochondrial | |
| L1968 | Succinyl-CoA ligase [GDP-forming] | 3514 |
| | alpha-chain, mitochondrial | |
| L1969 | Succinyl-CoA ligase [GDP-forming] | 3515 |
| | alpha-chain, mitochondrial | |
| L1970 | Succinyl-CoA ligase [GDP-forming] | 3516 |
| | alpha-chain, mitochondrial | |
| L1971 | Succinyl-CoA ligase [GDP-forming] | 3517 |
| | alpha-chain, mitochondrial | |
| L1972 | Succinyl-CoA synthetase beta chain | ADG |
| L1973 | Succinyl-CoA synthetase beta chain | RQP |
| L1974 | Succinyl-CoA synthetase beta chain | 3518 |
| L1975 | Succinyl-CoA synthetase beta chain | 3519 |
| L1976 | Succinyl-CoA synthetase beta chain | 3520 |
| L1977 | Succinyl-CoA synthetase beta chain | 3521 |
| L1978 | Succinyl-CoA synthetase beta chain | 3522 |
| L1979 | Succinyl-CoA synthetase beta chain | 3523 |
| L1980 | Succinyl-CoA:3-ketoacid-coenzyme A transferase | 3524 |
| L1981 | Sulfurtransferase | 3525 |
| L1982 | Superantigen SMEZ-2 | 3526 |
| L1983 | Superoxide dismutase 1 copper chaperone | 3527 |
| L1984 | Surface layer protein | 3528 |
| L1985 | Surface layer protein | 3529 |
| L1986 | Surface layer protein | 3530 |
| L1987 | Surface layer protein | 3531 |
| L1988 | Surface layer protein | 3532 |
| L1989 | Surface layer protein | 3533 |
| L1990 | Surface layer protein | 3534 |
| L1991 | Surface layer protein | 3535 |
| L1992 | T lymphocyte activation antigen | 3536 |
| L1993 | T lymphocyte activation antigen | 3537 |
| L1994 | T-cell receptor alpha chain C region | 3538 |
| L1995 | Terminal oxygenase component of carbazole | 3539 |
| L1996 | Tetanus neurotoxin | 3540 |
| L1997 | Tetracycline repressor protein class D | 3541 |
| L1998 | The GTP-binding protein Obg | 3542 |
| L1999 | The GTP-binding protein Obg | 3543 |
| L2000 | The GTP-binding protein Obg | 3544 |
| L2001 | The GTP-binding protein Obg | 3545 |
| L2002 | Thioredoxin domain-containing protein 4 | 3546 |
| L2003 | Thioredoxin domain-containing protein 4 | 3547 |
| L2004 | Thiosulfate sulfurtransferase | IDP |
| L2005 | Thiosulfate sulfurtransferase | 3548 |
| L2006 | Thiosulfate sulfurtransferase | 3549 |
| L2007 | Thiosulfate sulfurtransferase | 3550 |
| L2008 | Thiosulfate sulfurtransferase | 3551 |
| L2009 | Threonyl-tRNA synthetase | 3552 |
| L2010 | Threonyl-tRNA synthetase | 3553 |
| L2011 | Threonyl-tRNA synthetase | 3554 |
| L2012 | Threonyl-tRNA synthetase | 3555 |
| L2013 | Threonyl-tRNA synthetase | 3556 |
| L2014 | Threonyl-tRNA synthetase | 3557 |
| L2015 | Threonyl-tRNA synthetase | 3558 |
| L2016 | Threonyl-tRNA synthetase | 3559 |
| L2017 | Threonyl-tRNA synthetase | 3560 |
| L2018 | Threonyl-tRNA synthetase 1 | 3561 |
| L2019 | Threonyl-tRNA synthetase 1 | 3562 |
| L2020 | Threonyl-tRNA synthetase 1 | 3563 |
| L2021 | Threonyl-tRNA synthetase 1 | 3564 |
| L2022 | Threonyl-tRNA synthetase 1 | 3565 |
| L2023 | Threonyl-tRNA synthetase 1 | 3566 |
| L2024 | Threonyl-tRNA synthetase 1 | 3567 |
| L2025 | Threonyl-tRNA synthetase 1 | 3568 |
| L2026 | Thrombospondin 1 | 3569 |
| L2027 | Tick-borne encephalitis virus glycoprotein | 3570 |
| L2028 | Titin | 3571 |
| L2029 | Titin | 3572 |
| L2030 | TLR1789 protein | 3573 |
| L2031 | TLR1789 protein | 3574 |
| L2032 | Topoisomerase I | 3575 |
| L2033 | Topoisomerase 1 | 3576 |
| L2034 | Toxic shock syndrome toxin-1 | 3577 |
| L2035 | Toxic shock syndrome toxin-1 | 3578 |
| L2036 | Toxic shock syndrome toxin-1 | 3579 |
| L2037 | Toxic shock syndrome toxin-1 | 3580 |
| L2038 | T-plasminogen activator F1-G | VPV |
| L2039 | T-plasminogen activator F1-G | 3581 |
| L2040 | TpsB transporter FhaC | 3582 |
| L2041 | TpsB transporter FhaC | 3583 |
| L2042 | TpsB transporter FhaC | 3584 |
| L2043 | Transcarbamylase | 3585 |
| L2044 | Transcarbamylase | 3586 |
| L2045 | Transcription antiterminator LicT | 3587 |
| L2046 | Transcription elongation factor GreB | 3588 |
| L2047 | Transcription initiation factor IIa gamma chain | 3589 |
| L2048 | Transcription initiation factor IIb | 3590 |
| L2049 | Transcription initiation factor IIb | 3591 |
| L2050 | Transcriptional regulator (NtrC family) | 3592 |
| L2051 | Transcriptional regulator AefR | 3593 |
| L2052 | Transcriptional regulator AefR | 3594 |
| L2053 | Transcriptional regulator AefR | 3595 |
| L2054 | Transcriptional regulator AefR | 3596 |
| L2055 | Transcriptional regulator AefR | 3597 |
| L2056 | Transcriptional regulator, AsnC family | 3598 |
| L2057 | Transcriptional regulator, AsnC family | 3599 |
| L2058 | Transcriptional regulator, AsnC family | 3600 |
| L2059 | Transcriptional regulator, biotin repressor family | 3601 |
| L2060 | Transcriptional regulator, Crp/Fnr family | 3602 |
| L2061 | Transcriptional regulator, GntR family | 3603 |
| L2062 | Transcriptional regulator, HTH_3 family | 3604 |
| L2063 | Transcriptional regulator, HTH_3 family | 3605 |
| L2064 | Transcriptional regulator, HTH_3 family | 3606 |
| L2065 | Transcriptional regulator, HTH_3 family | 3607 |
| L2066 | Transcriptional regulator, HTH_3 family | 3608 |
| L2067 | Transcriptional regulator, laci family | 3609 |
| L2068 | Transcriptional regulatory protein ZraR | 3610 |
| L2069 | Transcriptional regulatory protein ZraR | 3611 |
| L2070 | Transcriptional regulatory protein ZraR | 3612 |
| L2071 | Transcriptional regulatory protein ZraR | 3613 |
| L2072 | Transcriptional regulatory protein ZraR | 3614 |
| L2073 | Transcriptional regulatory protein ZraR | 3615 |
| L2074 | Transcriptional regulatory protein ZraR | 3616 |
| L2075 | Transferrin receptor protein | VSN |
| L2076 | Transferrin receptor protein | 3617 |
| L2077 | Transferrin receptor protein | 3618 |
| L2078 | Transferrin receptor protein | 3619 |
| L2079 | Transferrin receptor protein | 3620 |
| L2080 | Translation initiation factor 5A | 3621 |
| L2081 | Translation initiation factor 5A | 3622 |
| L2082 | Translation initiation factor 5A | 3623 |
| L2083 | Translation initiation factor IF2/eIF5b | 3624 |
| L2084 | Translation initiation factor IF2/eIF5b | 3625 |
| L2085 | Transposable element mariner, complete CDS | 3626 |
| L2086 | Tricorn protease | 3627 |
| L2087 | Tricorn protease | 3628 |
| L2088 | Tricom protease | 3629 |
| L2089 | Trigger factor | 3630 |
| L2090 | Trigger factor | 3631 |
| L2091 | Trigger factor | 3632 |
| L2092 | TRNA CCA-adding enzyme | RRI |
| L2093 | TRNA CCA-adding enzyme | 3633 |
| L2094 | TRNA CCA-adding enzyme | 3634 |
| L2095 | TRNA CCA-adding enzyme | 3635 |
| L2096 | TRNA CCA-adding enzyme | 3636 |
| L2097 | TRNA nucleotidyltransferase | 3637 |
| L2098 | TRNA-splicing endonuclease | 3638 |
| L2099 | Tt1467 protein | LEA |
| L2100 | Tt1467 protein | 3639 |
| L2101 | Tumor suppressor p53-binding protein 1 | 3640 |
| L2102 | Tumor suppressor p53-binding protein 1 | 3641 |
| L2103 | Tumor suppressor p53-binding protein 1 | 3642 |
| L2104 | Tumor suppressor p53-binding protein 1 | 3643 |
| L2105 | Type A flavoprotein FprA | 3644 |
| L2106 | Type A flavoprotein FprA | 3645 |
| L2107 | Type A flavoprotein FprA | 3646 |
| L2108 | Type A flavoprotein FprA | 3647 |
| L2109 | Type A flavoprotein FprA | 3648 |
| L2110 | Type I restriction enzyme specificity protein MG438 | QMH |
| L2111 | Type I restriction enzyme specificity protein MG438 | 3649 |
| L2112 | Type I restriction enzyme specificity protein MG438 | 3650 |
| L2113 | Type I restriction-modification enzyme, S subunit | 3651 |
| L2114 | Type I restriction-modification enzyme, S subunit | 3652 |
| L2115 | Type I site-specific restriction- | 3653 |
| | modification system, R (restriction) subunit | |
| L2116 | Type I site-specific restriction- | 3654 |
| | modification system, R (restriction) subunit | |
| L2117 | Type I site-specific restriction- | 3655 |
| | modification system, R (restriction) subunit | |
| L2118 | Type II DNA topoisomerase VI subunit B | 3656 |
| L2119 | Type II DNA topoisomerase VI subunit B | 3657 |
| L2120 | Type II DNA topoisomerase VI subunit B | 3658 |
| L2121 | Type II DNA topoisomerase VI subunit B | 3659 |
| L2122 | Type II DNA topoisomerase VI subunit B | 3660 |
| L2123 | Type II DNA topoisomerase VI subunit B | 3661 |
| L2124 | Type II DNA topoisomerase VI subunit B | 3662 |
| L2125 | Type II DNA topoisomerase VI subunit B | 3663 |
| L2126 | Type II DNA topoisomerase VI subunit B | 3664 |
| L2127 | Type II DNA topoisomerase VI subunit B | 3665 |
| L2128 | Type II DNA topoisomerase VI subunit B | 3666 |
| L2129 | Type VI secretion system component | 3667 |
| L2130 | Type VI secretion system component | 3668 |
| L2131 | Type VI secretion system component | 3669 |
| L2132 | Tyrosine-protein kinase receptor UFO | 3670 |
| L2133 | Tyrosine-protein kinase receptor UFO | 3671 |
| L2134 | Tyrosine-protein kinase ZAP-70 | 3672 |
| L2135 | Tyrosine-protein kinase ZAP-70 | 3673 |
| L2136 | Tyrosyl-DNA phosphodiesterase | 3674 |
| L2137 | Tyrosyl-DNA phosphodiesterase | 3675 |
| L2138 | Ubiquitin carboxyl-terminal hydrolase 7 | 3676 |
| L2139 | UDP-galactopyranose mutase | 3677 |
| L2140 | UDP-galactopyranose mutase | 3678 |
| L2141 | UDP-galactopyranose mutase | 3679 |
| L2142 | UDP-galactopyranose mutase | 3680 |
| L2143 | UDP-galactopyranose mutase | 3681 |
| L2144 | UDP-glucose dehydrogenase | 3682 |
| L2145 | UDP-N-acetylmuramate-L-alanine ligase | 3683 |
| L2146 | UDP-N-acetylmuramate-L-alanine ligase | 3684 |
| L2147 | UDP-N-acetylmuramoylalanine—D-glutamate ligase | 3685 |
| L2148 | UDP-N-acetylmuramoylalanine—D-glutamate ligase | 3686 |
| L2149 | UDP-N-acetylmuramoylalanine- | 3687 |
| | D-glutamyl-lysine-D-alanyl-D-alanine | |
| | ligase, MurF protein | |
| L2150 | UDP-N-acetylmuramoylalanyl- | 3688 |
| | D-glutamate—2,6-diaminopimelate ligase | |
| L2151 | UDP-N-acetylmuramoylalanyl- | 3689 |
| | D-glutamate—2,6-diaminopimelate ligase | |
| L2152 | UDP-N-acetylmuramoylalanyl- | 3690 |
| | D-glutamate—2,6-diaminopimelate ligase | |
| L2153 | UDP-N-acetylmuramoylalanyl- | 3691 |
| | D-glutamate—2,6-diaminopimelate ligase | |
| L2154 | UDP-N-acetylmuramoylalanyl- | 3692 |
| | D-glutamate—2,6-diaminopimelate ligase | |
| L2155 | UDP-N-acetylmuramoylalanyl- | 3693 |
| | D-glutamate—2,6-diaminopimelate ligase | |
| L2156 | UDP-N-acetylmuramoylalanyl- | 3694 |
| | D-glutamate—2,6-diaminopimelate ligase | |
| L2157 | Uncharacterized conserved protein | 3695 |
| L2158 | Uncharacterized conserved protein | 3696 |
| L2159 | Uncharacterized GST-like protein yfcF | 3697 |
| L2160 | Uncharacterized GST-like proteinprotein | 3698 |
| L2161 | Uncharacterized GST-like proteinprotein | 3699 |
| L2162 | Uncharacterized GST-like proteinprotein | 3700 |
| L2163 | Uncharacterized protein | 3701 |
| L2164 | Uncharacterized protein | 3702 |
| L2165 | Uncharacterized protein BT 1490 | 3703 |
| L2166 | Uncharacterized protein ypfl | TLR |
| L2167 | Uncharacterized protein ypfl | VHP |
| L2168 | Uncharacterized protein ypfl | 3704 |
| L2169 | Uncharacterized protein ypfl | 3705 |
| L2170 | Uncharacterized protein ypfl | 3706 |
| L2171 | Uncharacterized protein ypfl | 3707 |
| L2172 | Uncharacterized protein ypfl | 3708 |
| L2173 | Uncharacterized protein ypfl | 3709 |
| L2174 | Uncharacterized protein ypfl | 3710 |
| L2175 | Uncharacterized protein ypfl | 3711 |
| L2176 | Uncharacterized protein ypfl | 3712 |
| L2177 | Uncharacterized protein ypfl | 3713 |
| L2178 | Uncharacterized protein ypfl | 3714 |
| L2179 | Uncharacterized protein ypfl | 3715 |
| L2180 | Uncharacterized protein ypfl | 3716 |
| L2181 | Uncharacterized protein ypfl | 3717 |
| L2182 | Uncharacterized protein ypfl | 3718 |
| L2183 | Unknown protein | 3719 |
| L2184 | Unknown protein | 3720 |
| L2185 | UPF0131 protein ykqA | 3721 |
| L2186 | UPF0131 protein ykqA | 3722 |
| L2187 | UPF0131 protein ykqA | 3723 |
| L2188 | UPF0348 protein MJ0951 | 3724 |
| L2189 | UPF0348 protein MJ0951 | 3725 |
| L2190 | UPF0348 protein MJ0951 | 3726 |
| L2191 | UPF0348 protein MJ0951 | 3727 |
| L2192 | UPF0348 protein MJ0951 | 3728 |
| L2193 | UPF0348 protein MJ0951 | 3729 |
| L2194 | UPF0348 protein MJ0951 | 3730 |
| L2195 | UPF0348 protein MJ0951 | 3731 |
| L2196 | URE2 protein | 3732 |
| L2197 | Uridine diphospho-N- | TAK |
| | acetylenolpyruvylglucosaminereductase | |
| L2198 | Uridine diphospho-N- | 3733 |
| | acetylenolpyruvylglucosaminereductase | |
| L2199 | Uridine diphospho-N- | 3734 |
| | acetylenolpyruvylglucosaminereductase | |
| L2200 | Uridine diphospho-N- | 3735 |
| | acetylenolpyruvylglucosaminereductase | |
| L2201 | Uridine diphospho-N- | 3736 |
| | acetylenolpyruvylglucosaminereductase | |
| L2202 | Urokinase plasminogen activator surface receptor | 3737 |
| L2203 | Urokinase plasminogen activator surface receptor | 3738 |
| L2204 | Vascular cell adhesion molecule-1 | 3739 |
| L2205 | VCP-like ATPase | 3740 |
| L2206 | VCP-like ATPase | 3741 |
| L2207 | Viral CASP8 and FADD-like apoptosis regulator | 3742 |
| L2208 | Vitamin K-dependent protein Z | 3743 |
| L2209 | VP1 protein | 3744 |
| L2210 | V-type ATP synthase alpha chain | 3745 |
| L2211 | Xaa-Pro aminopeptidase | 3746 |
| L2212 | Xaa-Pro aminopeptidase | 3747 |
| L2213 | Xaa-Pro aminopeptidase | 3748 |
| L2214 | Xaa-Pro aminopeptidase | 3749 |
| L2215 | Xanthine dehydrogenase | 3750 |
| L2216 | Xanthine dehydrogenase | 3751 |
| L2217 | Xanthine dehydrogenase | 3752 |
| L2218 | Xanthine dehydrogenase | 3753 |
| L2219 | X-prolyl dipeptidyl aminopeptidase | KSY |
| L2220 | X-prolyl dipeptidyl aminopeptidase | LDG |
| L2221 | X-prolyl dipeptidyl aminopeptidase | LLE |
| L2222 | X-prolyl dipeptidyl aminopeptidase | TYS |
| L2223 | X-prolyl dipeptidyl aminopeptidase | 3754 |
| L2224 | X-prolyl dipeptidyl aminopeptidase | 3755 |
| L2225 | X-prolyl dipeptidyl aminopeptidase | 3756 |
| L2226 | X-prolyl dipeptidyl aminopeptidase | 3757 |
| L2227 | X-prolyl dipeptidyl aminopeptidase | 3758 |
| L2228 | X-prolyl dipeptidyl aminopeptidase | 3759 |
| L2229 | X-prolyl dipeptidyl aminopeptidase | 3760 |
| L2230 | X-prolyl dipeptidyl aminopeptidase | 3761 |
| L2231 | X-prolyl dipeptidyl aminopeptidase | 3762 |
| L2232 | X-prolyl dipeptidyl aminopeptidase | 3763 |
| L2233 | X-prolyl dipeptidyl aminopeptidase | 3764 |
| L2234 | X-prolyl dipeptidyl aminopeptidase | 3765 |
| L2235 | X-prolyl dipeptidyl aminopeptidase | 3766 |
| L2236 | X-prolyl dipeptidyl aminopeptidase | 3767 |
| L2237 | X-prolyl dipeptidyl aminopeptidase | 3768 |
| L2238 | X-prolyl dipeptidyl aminopeptidase | 3769 |
| L2239 | X-prolyl dipeptidyl aminopeptidase | 3770 |
| L2240 | X-prolyl dipeptidyl aminopeptidase | 3771 |
| L2241 | X-prolyl dipeptidyl aminopeptidase | 3772 |
| L2242 | X-prolyl dipeptidyl aminopeptidase | 3773 |
| L2243 | X-prolyl dipeptidyl aminopeptidase | 3774 |
| L2244 | X-prolyl dipeptidyl aminopeptidase | 3775 |
| L2245 | X-prolyl dipeptidyl aminopeptidase | 3776 |
| L2246 | X-prolyl dipeptidyl aminopeptidase | 3777 |
| L2247 | Xylosidase/arabinosidase | 3778 |
| L2248 | Xylosidase/arabinosidase | 3779 |
| L2249 | Xylosidase/arabinosidase | 3780 |
| L2250 | Xylosidase/arabinosidase | 3781 |
| L2251 | Xylosidase/arabinosidase | 3782 |
| L2252 | Xylosidase/arabinosidase | 3783 |
| L2253 | Xylosidase/arabinosidase | 3784 |
| L2254 | YkoF | 3785 |
| L2255 | YkuI protein | 3786 |

Internal ribosomal entry site (IRES) is a nucleotide sequence (>500 nucleotides) that allows for initiation of translation in the middle of an mRNA sequence (Kim, J. H. et al., 2011. PLoS One 6(4): e18556; the contents of which are herein incorporated by reference in its entirety). Use of an IRES sequence ensures co-expression of genes before and after the IRES, though the sequence following the IRES may be transcribed and translated at lower levels than the sequence preceding the RES sequence.

2A peptides are small “self-cleaving” peptides (18-22 amino acids) derived from viruses such as foot-and-mouth disease virus (F2A), porcine teschovirus-1 (P2A), Thoseaasigna virus (T2A), or equine rhinitis A virus (E2A). The 2A designation refers specifically to a region of picornavirus polyproteins that lead to a ribosomal skip at the glycyl-prolyl bond in the C-terminus of the 2A peptide (Kim, J. H. et al., 2011. PLoS One 6(4): el 8556; the contents of which are herein incorporated by reference in its entirety). This skip results in a cleavage between the 2A peptide and its immediate downstream peptide. As opposed to IRES linkers, 2A peptides generate stoichiometric expression of proteins flanking the 2A peptide and their shorter length can be advantageous in generating viral expression vectors.

Some payload regions encode linkers comprising furin cleavage sites. Furin is a calcium dependent serine endoprotease that cleaves proteins just downstream of a basic amino acid target sequence (Arg-X-(Arg/Lys)-Arg) (Thomas, G., 2002. Nature Reviews Molecular Cell Biology 3(10): 753-66; the contents of which are herein incorporated by reference in its entirety). Furin is enriched in the trans-golgi network where it is involved in processing cellular precursor proteins. Furin also plays a role in activating a number of pathogens. This activity can be taken advantage of for expression of polypeptides of the disclosure.

In some embodiments, the payload region may encode one or more linkers comprising cathepsin, matrix metalloproteinases or legumain cleavage sites. Such linkers are described e.g. by Cizeau and Macdonald in International Publication No. WO2008052322, the contents of which are herein incorporated in their entirety. Cathepsins are a family of proteases with unique mechanisms to cleave specific proteins. Cathepsin B is a cysteine protease and cathepsin D is an aspartyl protease. Matrix metalloproteinases are a family of calcium-dependent and zinc-containing endopeptidases. Legumain is an enzyme catalyzing the hydrolysis of (-Asn-Xaa-) bonds of proteins and small molecule substrates.

In some embodiments, payload regions may encode linkers that are not cleaved. Such linkers may include a simple amino acid sequence, such as a glycine rich sequence. In some cases, linkers may comprise flexible peptide linkers comprising glycine and serine residues. The linker may comprise flexible peptide linkers of different lengths, e.g. nxG4S, where n=1-10 (SEQ ID NO: 32690) and the length of the encoded linker varies between 5 and 50 amino acids. In a non-limiting example, the linker may be 5xG4S (SEQ ID NO: 32689). These flexible linkers are small and without side chains so they tend not to influence secondary protein structure while providing a flexible linker between antibody segments (George, R. A., et al., 2002. Protein Engineering 15(11): 871-9; Huston, J. S. et al., 1988. PNAS 85:5879-83; and Shan, D. et al., 1999. Journal of Immunology. 162(11):6589-95; the contents of each of which are herein incorporated by reference in their entirety). Furthermore, the polarity of the serine residues improves solubility and prevents aggregation problems.

In some embodiments, payload regions of the disclosure may encode small and unbranched serine-rich peptide linkers, such as those described by Huston et al. in U.S. Pat. No. 5,525,491, the contents of which are herein incorporated in their entirety. Polypeptides encoded by the payload region of the disclosure, linked by serine-rich linkers, have increased solubility.

In some embodiments, payload regions of the disclosure may encode artificial linkers, such as those described by Whitlow and Filpula in U.S. Pat. No. 5,856,456 and Ladner et al. in U.S. Pat. No. 4,946,778, the contents of each of which are herein incorporated by their entirety.

In some embodiments, the payload region encodes at least one G-453 linker (e.g., SEQ ID NO: 1734 or SEQ ID NO: 2449).

In some embodiments, the payload region encodes at least one G4S linker (e.g., SEQ ID NO: 1733 or SEQ ID NO: 2443).

In some embodiments, the payload region encodes at least one furin site.

In some embodiments, the payload region encodes at least one T2A linker.

In some embodiments, the payload region encodes at least one F2A linker.

In some embodiments, the payload region encodes at least one P2A linker.

In some embodiments, the payload region encodes at least one IDES sequence.

In some embodiments, the payload region encodes at least one G4S5 linker (e.g., SEQ ID NO: 1728 or SEQ ID NO: 32689).

In some embodiments, the payload region encodes at least one furin and one 2A linker.

In some embodiments, the payload region encodes at least one hinge region. As a non-limiting example, the hinge is a IgG hinge.

In some embodiments, the linker region may be 1-50, 1-100, 50-100, 50-150, 100-150, 100-200, 150-200, 150-250, 200-250, 200-300, 250-300, 250-350, 300-350, 300-400, 350-400, 350-450, 400-450, 400-500, 450-500, 450-550, 500-550, 500-600, 550-600, 550-650, or 600-650 nucleotides in length. The linker region may have a length of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73,74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 115, 120, 125, 130, 135, 140, 145, 150, 151, 152, 153, 154, 155, 156, 157, 158, 159, 160, 165, 170, 171, 172, 173, 174, 175, 176, 177, 178, 179, 180, 185, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 640, 650 or greater than 650. In some embodiments, the linker region may be 12 nucleotides in length. In some embodiments, the linker region may be 18 nucleotides in length. In some embodiments, the linker region may be 45 nucleotides in length. In some embodiments, the linker region may be 54 nucleotides in length. In some embodiments, the linker region may be 66 nucleotides in length. In some embodiments, the linker region may be 75 nucleotides in length. In some embodiments, the linker region may be 78 nucleotides in length. In some embodiments, the linker region may be 87 nucleotides in length. In some embodiments, the linker region may be 108 nucleotides in length. In some embodiments, the linker region may be 153 nucleotides in length. In some embodiments, the linker region may be 198 nucleotides in length. In some embodiments, the linker region may be 623 nucleotides in length.

### Viral Genome Component: Introns

In some embodiments, the payload region comprises at least one element to enhance the expression such as one or more introns or portions thereof. Non-limiting examples of introns include, MVM (67-97 bps), HX truncated intron 1 (300 bps), ii-globin SIS/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In some embodiments, the intron or intron portion may be 1-100, 100-500, 500-1000, or 1000-1500 nucleotides in length. The intron may have a length of 10, 15, 20, 30, 40, 50, 60, 70, 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or greater than 500. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500. In some embodiments, the intron may be 15 nucleotides in length. In some embodiments, the intron may be 32 nucleotides in length. In some embodiments, the intron may be 41 nucleotides in length. In some embodiments, the intron may be 53 nucleotides in length. In some embodiments, the intron may be 54 nucleotides in length. In some embodiments, the intron may be 59 nucleotides in length. In some embodiments, the intron may be 73 nucleotides in length. In some embodiments, the intron may be 102 nucleotides in length. In some embodiments, the intron may be 134 nucleotides in length. In some embodiments, the intron may be 168 nucleotides in length. In some embodiments, the intron may be 172 nucleotides in length. In some embodiments, the intron may be 347 nucleotides in length. In some embodiments, the intron may be 1074 nucleotides in length.

### AAV Production

The present disclosure provides methods for the generation of parvoviral particles, e.g. AAV particles, by viral genome replication in a viral replication cell for use in VA-DER systems and/or methods.

In accordance with the disclosure, the viral genome comprising a payload region encoding an antibody, an antibody-based composition or fragment thereof, will be incorporated into the AAV particle produced in the viral replication cell. Methods of making AAV particles are well known in the art and are described in e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO1999014354, WO1999015685, WO1999047691, WO2000055342, WO2000075353, and WO2001023597; Methods In Molecular Biology, ed. Richard, Humana. Press, NJ (1995); O'Reilly et al., Baculovirus Expression Vectors, k Laboratory Manual. Oxford Univ. Press (1994); Samulski et al., J. Vir. 63:3822-8 (1989); Kajigaya et al., Proc. Nat′l Acad. Sci. USA 88: 4646-50 (1991); Ruffing et al., J. Vir. 66:6922-30 (1992); Kimbauer et al., Vir., 219:37-44 (1996); Zhao et al., Vir. 272:382-93 (2000); the contents of each of which are herein incorporated by reference in their entirety. In some embodiments, the AAV particles are made using the methods described in WO2015191508, the contents of which are herein incorporated by reference in their entirety.

Viral replication cells commonly used for production of recombinant AV viral vectors include but are not limited to 293 cells, COS cells, HeLa cells, KB cells, and other mammalian cell lines as described in U.S. Pat. Nos. 6,156,303, 5,387,484, 5,741,683, 5,691,176, and 5,688,676; U.S. patent publication No. 2002/0081721, and International Patent Publication Nos. WO 00/47757; WO 00/24916, and WO 96/17947; the contents of each of which are herein incorporated by reference in their entireties.

In some embodiments, the present disclosure provides a method for producing an AAV particle having enhanced (increased, improved) transduction efficiency comprising the steps of: 1) co-transfecting competent bacterial cells with a bacmid vector and either a viral construct vector and/or AAV payload construct vector, 2) isolating the resultant viral construct expression vector and AAV payload construct expression vector and separately transfecting viral replication cells, 3) isolating and purifying resultant payload and viral construct particles comprising viral construct expression vector or AAV payload construct expression vector, 4) co-infecting a viral replication cell with both the AAV payload and viral construct particles comprising viral construct expression vector or AAV payload construct expression vector, and 5) harvesting and purifying the AAV particle comprising a viral genome.

In some embodiments, the present disclosure provides a method for producing an AAV particle comprising the steps of 1) simultaneously co-transfecting mammalian cells, such as, but not limited to HEK293 cells, with a payload region, a construct expressing rep and cap genes and a helper construct, and 2) harvesting and purifying the AAV particle comprising a viral genome.

In some embodiments, the viral genome of the AAV particle of the disclosure optionally encodes a selectable marker. The selectable marker may comprise a cell-surface marker, such as any protein expressed on the surface of the cell including, but not limited to receptors, CD markers, lectins, integrins, or truncated versions thereof.

In some embodiments, selectable marker reporter genes are selected from those described in International Application No. WO 96/23810; Heim et al., Current Biology 2:178-182 (1996); Heim et al., Proc. Natl. Acad. Sci. USA (1995); or Heim et al., Science 373:663-664 (1995); WO 96/30540, the contents of each of which are incorporated herein by reference in their entireties).

### Payloads of the Disclosure

Any of the delivery vehicles, e.g., retroviral. lentiviral. AAV, plasmid, etc of the present disclosure may comprise at least one payload region. As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi polypeptide or a modulatory nucleic acid or regulatory nucleic acid. Payloads of the present disclosure, when they encode amino acid based molecules, typically encode polypeptides (e.g., peptides, polypeptides, antibodies or antibody based compositions) or fragments or variants thereof.

The payload region may be constructed in such a way as to reflect a region similar to or mirroring the natural organization of an mRNA.

The payload region may comprise a combination of coding and non-coding nucleic acid sequences. Payloads may also be non-coding nucleic acid based molecules such as miRNA, siRNA, aptamers, ribozymes, etc.

In some embodiments, the payload region may encode a coding or non-coding RNA.

In some embodiments, where the delivery vehicle is an AAV, the AAV particle comprises a viral genome with a payload region comprising nucleic acid sequences encoding more than one polypeptide of interest (e.g., a protein such as TRIM21 and/or an antibody). In such an embodiment, a viral genome encoding more than one polypeptide may be replicated and packaged into a viral particle. A target cell transduced with a viral particle comprising more than one polypeptide may express each of the polypeptides in a single cell.

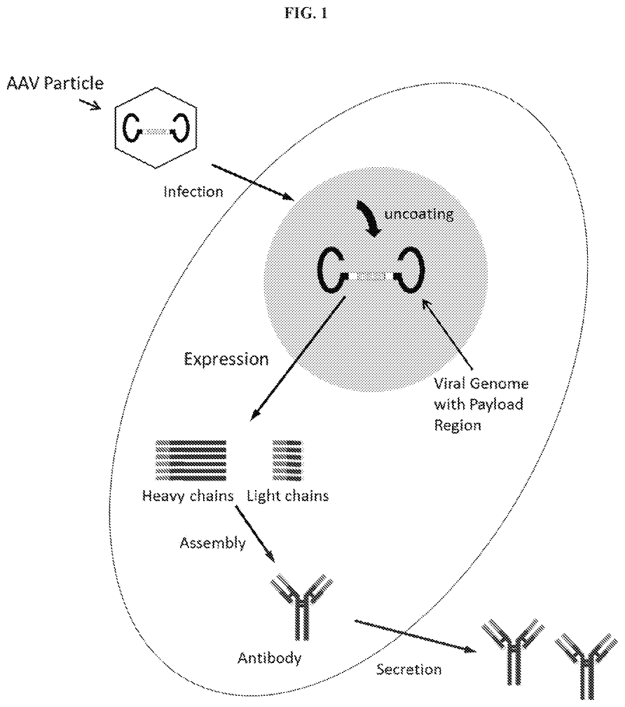

In some embodiments, as shown in FIG. 1, an AAV particle comprises a viral genome with a payload region comprising a nucleic acid sequence encoding a heavy chain and a light chain of an antibody. The heavy chain and light chain are expressed and assembled to form the antibody which is secreted.

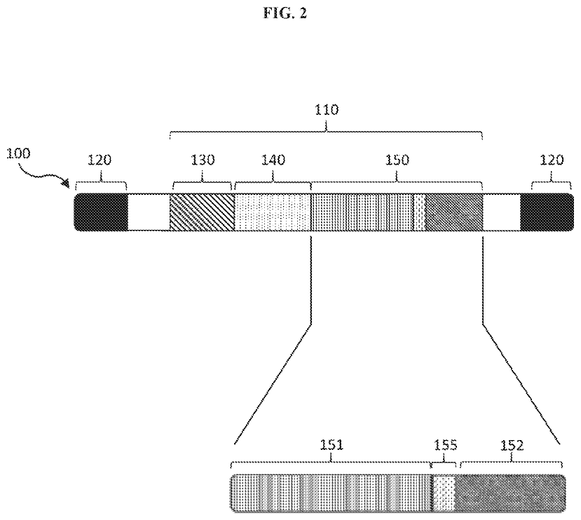

In some embodiments, the payload region may comprise the components as shown in FIG. 2. The payload region 110 is located within the viral genome 100. At the 5′ and/or the 3′ end of the payload region 110 there may be at least one inverted terminal repeat (ITR) 120. Within the payload region, there is a promoter region 130, an intron region 140 and a coding region 150. When the coding region 150 comprises a heavy chain region 151 and light chain region 152 of an antibody, the two chains may be separated by a linker region 155.

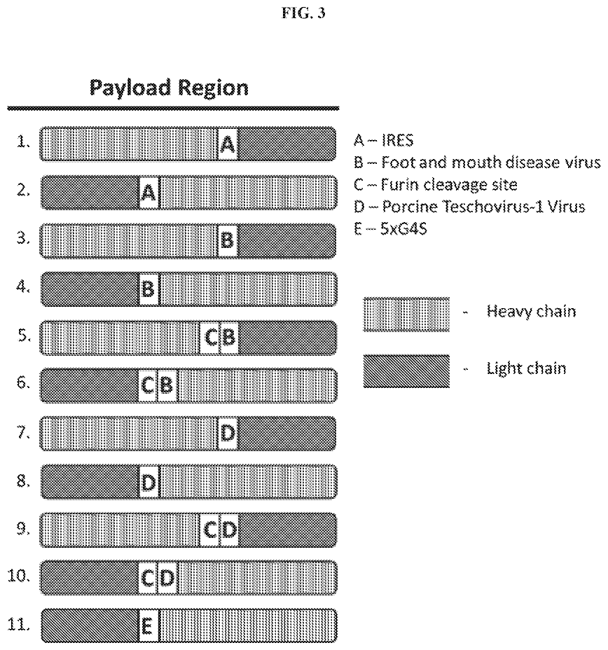

In some embodiments, the coding region may comprise a heavy and light chain sequence and a linker. As shown in FIG. 3, the payload region may comprise a heavy chain and light chain sequence separated by a linker and/or a cleavage site. In some embodiments, the heavy and light chain sequence is separated by an IRES sequence (1 and 2). In some embodiments, the heavy and light chain sequence is separated by a foot and mouth virus sequence (3 and 4). In some embodiments, the heavy and light chain sequence is separated by a foot and mouth virus sequence and a furin cleavage site (5 and 6). In some embodiments, the heavy and light chain sequence is separated by a porcine teschovirus-.1 virus sequence (7 and 8). In some embodiments, the heavy and light chain sequence is separated by a porcine teschovirus-1 virus and a furin cleavage site (9 and 10). In some embodiments, the heavy and light chain sequence is separated by a 5xG4S sequence (SEQ ID NO: 1728 or SEQ ID NO: 32689) (11).

Where the AAV particle payload region encodes a polypeptide, the polypeptide may be a peptide or protein. A protein encoded by the AAV particle payload region may comprise an antibody, an antibody related composition, a secreted protein, an intracellular protein, an extracellular protein, and/or a membrane protein. The encoded proteins may be structural or functional. In addition to the antibodies or antibody-based composition, proteins encoded by the payload region may include, in combination, certain mammalian proteins involved in immune system regulation. The AAV viral genomes encoding polypeptides described herein may be useful in the fields of human disease, viruses, infections veterinary applications and a variety of in vivo and in vitro settings.

In some embodiments, the AAV particles are useful in the field of medicine for the treatment, prophylaxis, palliation, or amelioration of neurological diseases and/or disorders.

### Antibodies and Antibody-Based Compositions

Payload regions of the viral particles of the disclosure may encode polypeptides that form one or more functional antibodies or antibody-based compositions. The phrase “viral particles” is used to refer to an AAV particle, lentiviral particle and/or a retroviral particle. As used herein, the term “antibody” is referred to in the broadest sense and specifically covers various embodiments including, but not limited to monoclonal antibodies, polyclonal antibodies, multispecific antibodies (e.g. bispecific antibodies formed from at least two intact antibodies), and antibody fragments (e.g., diabodies) so long as they exhibit a desired biological activity (e.g., “functional”). Antibodies are primarily amino-acid based molecules but may also comprise one or more modifications (including, but not limited to the addition of sugar moieties, fluorescent moieties, chemical tags, etc.).

As used herein, “antibody-based” or “antibody-derived” compositions are monomeric or multi-merit polypeptides which comprise at least one amino-acid region derived from a known or parental antibody sequence and at least one amino acid region derived from a non-antibody sequence, e.g., mammalian protein.

Payload regions may encode polypeptides that form or function as any antibody, including antibodies that are known in the art and/or antibodies that are commercially available. The encoded antibodies may be therapeutic, diagnostic, or for research purposes. Further, polypeptides of the disclosure may include fragments of such antibodies or antibodies that have been developed to comprise one or more of such fragments (e.g., variable domains or complementarity determining regions (Mils)).

In some embodiments, the viral genome of the viral particles may comprise nucleic acids which have been engineered to enable expression of antibodies, antibody fragments, or components of any of those described in U.S. Pat. No. 7,041,807 related to VYX epitope; US20090175884, US20110305630; US20130330275 related to misfolded proteins in cancer; US20040175775 related to PrP in eye fluid; US20030114360 related to copolymers and methods of treating prion-related diseases; WO2009121176 related to insulin-induced gene peptide compositions; US20030022243, WO2003000853 related to protein aggregation assays; WO200078344 related to prion protein peptides and uses thereof. Each of these publications are incorporated by reference in their entireties.

### Antibody Generation

In some embodiments, viral genomes of the viral particles of the disclosure may encode antibodies or antibody-based compositions produced using methods known in the art. Such methods may include but are not limited to immunization and display technologies (e.g., phage display, yeast display, and ribosomal display). Antibodies may be developed, for example, using any naturally occurring or synthetic antigen. As used herein, an “antigen” is an entity which induces or evokes an immune response in an organism. An immune response is characterized by the reaction of the cells, tissues and/or organs of an organism to the presence of a foreign entity. Such an immune response typically leads to the production by the organism of one or more antibodies against the foreign entity, e.g., antigen or a portion of the antigen. As used herein, “antigens” also refer to binding partners for specific antibodies or binding agents in a display library.

In some embodiments, the sequences of the polypeptides to be encoded in the viral genomes of the disclosure may be derived from antibodies produced using hybridoma technology. Host animals (e.g. mice, rabbits, goats, and llamas) may be immunized by an injection with an antigenic protein to elicit lymphocytes that specifically bind to the antigen. Lymphocytes may be collected and fused with immortalized cell lines to generate hybridomas which can be cultured in a suitable culture medium to promote growth. The antibodies produced by the cultured hybridomas may be subjected to analysis to determine binding specificity of the antibodies for the target antigen. Once antibodies with desirable characteristics are identified, corresponding hybridomas may be subcloned through limiting dilution procedures and grown by standard methods. The antibodies produced by these cells may be isolated and purified using standard immunoglobulin purification procedures.

In some embodiments, the sequences of the polypeptides to be encoded in the viral genomes of the disclosure may be produced using heavy and light chain variable region cDNA sequences selected from hybridomas or from other sources. Sequences encoding antibody variable domains expressed by hybridomas may be determined by extracting RNA molecules from antibody-producing hybridoma cells and producing cDNA by reverse transcriptase polymerase chain reaction (PCR). PCR may be used to amplify cDNA using primers specific for heavy and light chain sequences. PCR products may then be subcloned into plasmids for sequence analysis. Antibodies may be produced by insertion of resulting variable domain sequences into expression vectors.

In some embodiments, the sequences of the polypeptides to be encoded in the viral genomes of the disclosure may be generated using display technologies. Display technologies used to generate polypeptides of the disclosure may include any of the display techniques (e.g. display library screening techniques) disclosed in International Patent Application No. WO2014074532 the contents of which are herein incorporated by reference in their entirety. In some embodiments, synthetic antibodies may be designed, selected, or optimized by screening target antigens using display technologies (e.g. phage display technologies). Phage display libraries may comprise millions to billions of phage particles, each expressing unique antibody fragments on their viral coats. Such libraries may provide richly diverse resources that may be used to select potentially hundreds of antibody fragments with diverse levels of affinity for one or more antigens of interest (McCafferty, et al., 1990. Nature. 348:552-4; Edwards, B. M. et al., 2003. 1 MB. 334: 103.18; Schofield, D. et al., 2007. Genome Biol. 8, 8254 and Pershad, K. et al., 2010. Protein Engineering Design and Selection. 23:279-88; the contents of each of which are herein incorporated by reference in their entirety). Often, the antibody fragments present in such libraries comprise scFv antibody fragments, comprising a fusion protein of V_{H} and V_{L} antibody domains joined by a flexible linker. In some cases, scFvs may contain the same sequence with the exception of unique sequences encoding variable loops of the CDRs. In some cases, scFvs are expressed as fusion proteins, linked to viral coat proteins (e.g. the N-terminus of the viral pill coat protein). V_{L} chains may be expressed separately for assembly with V_{H} chains in the periplasm prior to complex incorporation into viral coats. Precipitated library members may be sequenced from the bound phage to obtain cDNA encoding desired says. Antibody variable domains or CDRs from such sequences may be directly incorporated into antibody sequences for recombinant antibody production or mutated and utilized for further optimization through in vitro affinity maturation.

In some embodiments, the sequences of the polypeptides to be encoded in the viral genomes of the disclosure may be produced using yeast surface display technology, wherein antibody variable domain sequences may be expressed on the cell surface of Saccharomyces cerevisiae. Recombinant antibodies may be developed by displaying the antibody fragment of interest as a fusion to e.g. Aga2p protein on the surface of the yeast, where the protein interacts with proteins and small molecules in a solution says with affinity toward desired receptors may be isolated from the yeast surface using magnetic separation and flow cytometry. Several cycles of yeast surface display and isolation may be done to attain says with desired properties through directed evolution.

In some embodiments, the sequence of the polypeptides to be encoded in the viral genomes of the disclosure (e.g., antibodies) may be designed by VERSITOPE™ Antibody Generation and other methods used by BIOATLA® and described in United States Patent Publication No, US20130281303, the contents of which are herein incorporated by reference in their entirety. In brief recombinant monoclonal antibodies are derived from B-cells of a host immuno-challenged with one or more target antigens. These methods of antibody generation do not rely on immortalized cell lines, such as hybridoma, thereby avoiding some of the associated challenges i.e., genetic instability and low production capacity, producing high affinity and high diversity recombinant monoclonal antibodies. In some embodiments, the method is a natural diversity approach. In another embodiment, the method is a high diversity approach.

In some embodiments, the sequences of the polypeptides to be encoded in the viral genomes of the disclosure may be generated using the BIOATLA® natural diversity approach. In the natural diversity approach of generating recombinant monoclonal antibodies described in United States Patent Publication No. US20130281303, the original pairings of variable heavy (V_{H}) and variable light (V_{L}) domains are retained from the host, yielding recombinant monoclonal antibodies that are naturally paired. These may be advantageous due to a higher likelihood of functionality as compared to non-natural pairings of V_{H} and VL. To produce the recombinant monoclonal antibodies, first a non-human host (i.e., rabbit, mouse, hamster, guinea pig, camel or goat) is immuno-challenged with an antigen of interest. In some embodiments, the host may be a previously challenged human patient. In other embodiments, the host may not have been immuno-challenged, B-cells are harvested from the host and screened by fluorescence activated cell sorting (FACS), or other method, to create a library of B-cells enriched in B-cells capable of binding the target antigen. The cDNA obtained from the mRNA of a single B-cell is then amplified to generate an immunoglobulin library of V_{H} and VT, domains. This library of immunoglobulins is then cloned into expression vectors capable of expressing the V_{H} and V_{L}, domains, wherein the V_{H} and V_{L}, domains remain naturally paired. The library of expression vectors is then used in an expression system to express the V_{H} and V_{L} domains in order to create an antibody library. Screening of the antibody library yields antibodies able to bind the target antigen, and these antibodies can be further characterized. Characterization may include one or more of the following: isoelectric point, thermal stability, sedimentation rate, folding rate, neutralization or antigen activity, antagonist or agonistic activity, expression level, specific and non-specific binding, inhibition of enzymatic activity, rigidity/flexibility, shape, charge, stability across pH, in solvents, under UV radiation, in mechanical stress conditions, or in sonic conditions, half-life, and glycosylation.

In some embodiments, the sequences of the polypeptides to be encoded in the viral genomes of the disclosure may be generated using the BIOATLA® high diversity approach. In the high diversity approach of generating recombinant monoclonal antibodies described in United States Patent Publication No. US20130281303, additional pairings of variable heavy (NTH) and variable light (V_{L}) domains are attained. To produce the recombinant monoclonal antibodies, B-cells harvested from the host are screened by fluorescence activated cell sorting (FACS), panning, or other method, to create a library of B-cells enriched in B-cells capable of binding the target antigen. The cDNA obtained from the mRNA of the pooled B-cells is then amplified to generate an immunoglobulin library of V_{H} and AIL domains. This library of immunoglobulins is then used in a biological display system (mammalian, yeast or bacterial cell surface display systems) to generate a population of cells displaying antibodies, fragments or derivatives comprising the V_{H} and V_{L} domains wherein, the antibodies, fragments or derivatives comprise V_{H} and V_{L}, domain combinations that were not present in the B-cells in vivo. Screening of the cell population by FACS, with the target antigen, yields a subset of cells capable of binding the target antigen and the antibodies displayed on these cells can be further characterized. In an alternate embodiment of the high diversity approach, the immunoglobulin library comprises only V_{H} domains obtained from the B-cells of the immuno-challenged host, while the V_{L} domain(s) are obtained from another source.

In some embodiments, the sequences of the polypeptides to be encoded in the viral genomes of the disclosure may be evolved using BIOATLA® comprehensive approaches. The methods of generating recombinant monoclonal antibodies as described in United States Patent Publication No, US20130281303, further comprises evolving the recombinant antibody by comprehensive positional evolution (CPE™) CPE™ followed by comprehensive protein synthesis (CPS™), PCR shuffling, or other method.

In some embodiments, the sequence of the polypeptides to be encoded in the viral genomes of the disclosure (e.g., antibodies) may be derived from any of the BIOATLA® protein evolution methods described in International Publication WO2012009026, the contents of which are herein incorporated by reference in their entirety. In this method, mutations are systematically performed throughout the polypeptide or molecule of interest, a map is created providing useful informatics to guide the subsequent evolutionary steps. Not wishing to be bound by theory, these evolutionary methods typically start with a template polypeptide and a mutant is derived therefrom, which has desirable properties or characteristics. Non-limiting examples of evolutionary techniques include polymerase chain reaction (PCR), error prone PCR, oligonucleotide-directed mutagenesis, cassette mutagenesis, shuffling, assembly PCR, sexual PCR mutagenesis, in vivo mutagenesis, site-specific mutagenesis, gene reassembly, gene site saturated mutagenesis, in vitro mutagenesis, ligase chain reaction, oligonucleotide synthesis or any combination thereof.

In some embodiments, the BIOATLA® evolution method is Comprehensive Positional Evolution (CPE™). In CPE, naturally occurring amino acid variants are generated for each of the codons of the template polypeptide, wherein 63 different codon options exist for each amino acid variant. A set of polypeptides with single amino acid mutations are generated and the mutations are then confirmed by sequencing or other method known in the art and each amino acid change screened for improved function, neutral mutations, inhibitory mutations, expression, and compatibility with the host system. An EvoMap™ is created that describes in detail the effects of each amino acid mutation on the properties and characteristics of that polypeptide. The data from the EvoMap™ may be utilized to produce polypeptides with more than one amino acid mutation, wherein the resultant multi-site mutant polypeptides can be screened for desirable characteristics.

In some embodiments, the BIOATLA® evolution method is Synergy Evolution, wherein an EvoMap™ is used to identify amino acid positions to introduce 2-20 mutations simultaneously to produce a combinatorial effect. The resulting multi-site mutant polypeptides may be screened on one or more pre-determined characteristics to identify “upmutants” wherein the function of the mutant is improved as compared to the parent polypeptide. In some embodiments, Synergy Evolution is used to enhance binding affinity of an antibody.

In some embodiments, the BIOATLA® evolution method is Flex Evolution, wherein an EvoMap™ is used to identify fully mutable sites within a polypeptide that may then be targeted for alteration, such as introduction of glycosylation sites or chemical conjugation.

In some embodiments, the BIOATLA® evolution method is Comprehensive Positional Insertion Evolution (CPI™), wherein an amino acid is inserted after each amino acid of a template polypeptide to generate a set of lengthened polypeptides. CPI may be used to insert 1, 2, 3, 4, or 5 amino acids at each new position. The resultant lengthened polypeptides are sequenced and assayed for one or more pre-determined properties and evaluated in comparison to its template or parent molecule. In some embodiments, the binding affinity and immunogenicity of the resultant polypeptides are assayed. In some embodiments, the lengthened polypeptides are further mutated and mapped to identify polypeptides with desirable characteristics.

In some embodiments, the BIOATLA® evolution approach is Comprehensive Positional Deletion Evolution (CPD™), wherein each amino acid of the template polypeptide is individually and systematically deleted one at a time. The resultant shortened polypeptides are then sequenced and evaluated by assay for at least one predetermined feature. In some embodiments, the shortened polypeptides are further mutated and mapped to identify polypeptides with desirable characteristics.

In some embodiments, the BIOATLA® evolution approach is Combinatorial Protein Synthesis (CPS™), wherein mutants identified in CPE, CPI, CPD, or other evolutionary techniques are combined for polypeptide synthesis. These combined mutant polypeptides are then screened for enhanced properties and characteristics. In some embodiments CPS is combined with any of the aforementioned evolutionary or polypeptide synthesis methods.

In some embodiments, the sequence of the polypeptides to be encoded in the viral genomes of the disclosure (e.g., antibodies) may be derived from the BIOATLA® Comprehensive Integrated Antibody Optimization (CIAO!™) described in U.S. Pat. No. 8,859,467, the contents of which are herein incorporated by reference in their entirety. The CIAO!™ method allows for simultaneous evolution of polypeptide performance and expression optimization, within a eukaryotic cell host (i.e., mammalian or yeast cell host). First, an antibody library is generated in a mammalian cell production host by antibody cell surface display, wherein the generated antibody library targets a particular antigen of interest. The antibody library is then screened by any method known in the art, for one or more properties or characteristics. One or more antibodies of the library, with desirable properties or characteristics are chosen for further polypeptide evolution by any of the methods known in the art, to produce a library of mutant antibodies by antibody cell surface display in a mammalian cell production host. The generated mutant antibodies are screened for one or more predetermined properties or characteristics, whereby an upmutant is selected, wherein the upmutant has enhanced or improved characteristics as compared to the parent template polypeptide.

In some embodiments, the sequences of the polypeptides to be encoded in the viral genomes of the disclosure may be humanized by the methods of BIOATLA® as described in United States Patent Publication US20130303399, the contents of which are herein incorporated by reference in their entirety. In this method, for generating enhanced full length humanized antibodies in mammalian cells, no back-mutations are required to retain affinity to the antigen and no CDR grafting or phage-display is necessary. The generated humanized antibody has reduced immunogenicity and equal or greater affinity for the target antigen as compared to the parent antibody. The variable regions or CDRs of the generated humanized antibody are derived from the parent or template, whereas the framework and constant regions are derived from one or more human antibodies. To start, the parent, or template antibody is selected, cloned and each CDR sequence identified and synthesized into a CDR fragment library. Double stranded DNA fragment libraries for V_{H} and V_{L} are synthesized from the CDR fragment encoding libraries, wherein at least one CDR fragment library is derived from the template antibody and framework (FW) fragment encoding libraries, wherein the FW fragment library is derived from a pool of human frameworks obtained from natively expressed and functional human antibodies. Stepwise liquid phase ligation of FW and CDR encoding fragments is then used to generate both V_{H} and V_{L} fragment libraries. The V_{H} and V_{L} fragment libraries are then cloned into expression vectors to create a humanization library, which is further transfected into cells for expression of full length humanized antibodies, and used to create a humanized antibody library. The humanized antibody library is then screened to determine expression level of the humanized antibodies, affinity or binding ability for the antigen, and additional improved or enhanced characteristics, as compared to the template or parent antibody. Non-limiting examples of characteristics that may be screened include equilibrium dissociation constant (K_{D}), stability, melting temperature (Tₘ), pI, solubility, expression level, reduced immunogenicity, and improved effector function.

In some embodiments, the sequences of the polypeptides to be encoded in the viral genomes of the disclosure may be generated by the BIOATLA® method for preparing conditionally active antibodies as described in International Publications WO2016033331 and WO2016036916, the contents of which are herein incorporated by reference in their entirety. As used herein, the term “conditionally active” refers to a molecule that is active at an aberrant condition. Further, the conditionally active molecule may be virtually inactive at normal physiological conditions. Aberrant conditions may result from changes in pH, temperature, osmotic pressure, osmolality, oxidative stress, electrolyte concentration, and/or chemical or proteolytic resistance, as non-limiting examples.

The method of preparing a conditionally active antibody is described in International Publications WO2016033331 and WO2016036916 and summarized herein. Briefly, a wild-type polypeptide is selected and the DNA is evolved to create mutant DNAs. Non-limiting examples of evolutionary techniques that may be used to evolve the DNA include polymerase chain reaction (PCR), error prone PCR, shuffling, oligonucleotide-directed mutagenesis, assembly PCR, sexual PCR mutagenesis, in vivo mutagenesis, site-specific mutagenesis, gene reassembly, gene site saturated mutagenesis, in vitro mutagenesis, ligase chain reaction, oligonucleotide synthesis or any combination thereof. Once mutant DNAs are created, they are expressed in a eukaryotic cell production host (i.e., fungal. insect, mammalian, adenoviral. plant), wherein a mutant polypeptide is produced. The mutant polypeptide and the corresponding wild-type polypeptide are then subjected to assays under both normal physiological conditions and aberrant conditions in order to identify mutants that exhibit a decrease in activity in the assay at normal physiological conditions as compared to the wild-type polypeptide and/or an increase in activity in the assay under aberrant conditions, as compared to the corresponding wild-type polypeptide. The desired conditionally active mutant may then be produced in the aforementioned eukaryotic cell production host.

In some embodiments, the conditionally active antibody is a “mirac protein” as described by BIOATLA® in U.S. Pat. No. 8,709,755, the contents of which are herein incorporated by reference in their entirety. As used herein “mirac protein” refers to a conditionally active antibody that is virtually inactive at body temperature but active at lower temperatures.

In some embodiments, the sequence of the polypeptides to be encoded in the viral genomes of the disclosure (e.g., antibodies) may be derived based on any of the BIOATLA™ methods including, but not limited to, VERSITOPE™ Antibody Generation, natural diversity approaches, and high diversity approaches for generating monoclonal antibodies, methods for generation of conditionally active polypeptides, humanized antibodies, mirac proteins, multi-specific antibodies or cross-species active mutant polypeptides, Comprehensive Integrated Antibody Optimization (CIAO!™), Comprehensive Positional Evolution (CPE™), Synergy Evolution, Flex Evolution, Comprehensive Positional Insertion Evolution (CPI™), Comprehensive Positional Deletion Evolution (CPD™) Combinatorial Protein Synthesis (CPS™), or any combination thereof. These methods are described in U.S. Pat. Nos. 8,859,467 and 8,709,755 and United States Publication Nos. US20130281303, US20130303399, US20150065690, US20150252119, US20150086562 and US20100138945, and International Publication Nos. WO2015105888; WO2012009026, WO2011109726, WO2016036916, and WO2016033331, the contents of each of which are herein incorporated by reference in their entirety.

In some embodiments, antibodies of the present disclosure are generated by any of the aforementioned means to target one or more of the following epitopes of the tau protein; phosphorylated tau peptides, pS396, pS396-pS404, pS404, pS396-pS404-pS422, pS422, pS199, pS199-pS202, pS202, pT181, 07231, cis-pT231, any of the following acetylated sites acK174, acK274, acK280, acK281 and/or any combination thereof.

Antibody Fragments and Variants

In some embodiments, antibody fragments encoded by payloads of the disclosure comprise antigen binding regions from intact antibodies. Examples of antibody fragments may include, but are not limited to Fab, Fab′, F(ab)₂, and Fv fragments; diabodies; linear antibodies; single-chain antibody molecules; and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab)₂ fragment that has two antigen-binding sites and is still capable of cross-linking antigen. Compounds and/or compositions of the present disclosure may comprise one or more of these fragments. For the purposes herein, an “antibody” may comprise a heavy and light variable domain as well as an Fc region.

In some embodiments, the Fc region may be a modified Fe region, as described in US Patent Publication US20150065690, wherein the Fc region may have a single amino acid substitution as compared to the corresponding sequence for the wild-type Fc region, wherein the single amino acid substitution yields an Fc region with preferred properties to those of the wild-type Fc region. Non-limiting examples of Fe properties that may be altered by the single amino acid substitution include bind properties or response to pH conditions

As used herein, the term “native antibody” refers to an usually heterotetrameric glycoprotein of about 150,000 Daltons, composed of two identical light (L) chains and two identical heavy (H) chains. Genes encoding antibody heavy and light chains are known and segments making up each have been well characterized and described Matsuda, F. et al., 1998. The journal of Experimental Medicine. 188(11); 2151-62 and Li, A. et al., 2004. Blood. 103(12: 4602-9, the content of each of which are herein incorporated by reference in their entirety). Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain.

As used herein, the term “variable domain” refers to specific antibody domains found on both the antibody heavy and light chains that differ extensively in sequence among antibodies and are used in the binding and specificity of each particular antibody for its particular antigen. Variable domains comprise hypervariable regions. As used herein, the term “hypervariable region” refers to a region within a variable domain comprising amino acid residues responsible for antigen binding. The amino acids present within the hypervariable regions determine the structure of the complementarity determining regions (CDRs) that become part of the antigen-binding site of the antibody. As used herein, the term “CDR” refers to a region of an antibody comprising a structure that is complimentary to its target antigen or epitope. Other portions of the variable domain, not interacting with the antigen, are referred to as framework (FW) regions. The antigen-binding site (also known as the antigen combining site or paratope) comprises the amino acid residues necessary to interact with a particular antigen. The exact residues making up the antigen-binding site are typically elucidated by co-crystallography with bound antigen, however computational assessments can also be used based on comparisons with other antibodies (Strohl, W.R. Therapeutic Antibody Engineering. Woodhead Publishing, Philadelphia PA. 2012. Ch. 3, p 47-54, the contents of which are herein incorporated by reference in their entirety). Determining residues making up CDRs may include the use of numbering schemes including, but not limited to, those taught by Kabat (Wu, T. T. et al., 1970, JEM, 132(2):211-50 and Johnson, G. et al., 2000, Nucleic Acids Res. 28(1): 214-8, the contents of each of which are herein incorporated by reference in their entirety), Chothia (Chothia and Lesk, J. Mol, Biol, 196, 901 (1987), Chothia et al., Nature 342, 877 (1989) and A1-Lazikani, B. et al., 1997, J. Mol. Biol. 273(4):927-48, the contents of each of which are herein incorporated by reference in their entirety), Lefranc (Lefranc, M. P. et al., 2005, Immunome Res. 1:3) and Honegger (Honegger, A. and Pluckthun, A. 2001, J. Mol, Biol. 309(3):657-70, the contents of which are herein incorporated by reference in their entirety).

V_{H} and V_{L} domains have three CDRs each. V_{L} CDRs are referred to herein as CDR-Li, and CDR-L3, in order of occurrence when moving from N- to C-terminus along the variable domain polypeptide. V_{H} CDRs are referred to herein as CDR-H1, CDR-H2, and CDR-F13, in order of occurrence when moving from N- to C-terminus along the variable domain polypeptide. Each of CDRs have favored canonical structures with the exception of the CDR-H3, which comprises amino acid sequences that may be highly variable in sequence and length between antibodies resulting in a variety of three-dimensional structures in antigen-binding domains (Nikoloudis, D. et al., 2014. Peer J. 2:0,456, the contents of which are herein incorporated by reference in their entirety). In some cases, CDR-H3s may be analyzed among a panel of related antibodies to assess antibody diversity. Various methods of determining CDR sequences are known in the art and may be applied to known antibody sequences (Strohl, W. R. Therapeutic Antibody Engineering. Woodhead Publishing, Philadelphia PA. 2012. Ch. 3, p47-54, the contents of which are herein incorporated by reference in their entirety).

As used herein, the term “Fry” refers to an antibody fragment comprising the minimum fragment on an antibody needed to form a complete antigen-binding site. These regions consist of a dimer of one heavy chain and one light chain variable domain in tight, non-covalent association. Fv fragments can be generated by proteolytic cleavage, but are largely unstable. Recombinant methods are known in the art for generating stable Fv fragments, typically through insertion of a flexible linker between the light chain variable domain and the heavy chain variable domain [to form a single chain Fv (scFv)] or through the introduction of a disulfide bridge between heavy and light chain variable domains (Strohl, W. R. Therapeutic Antibody Engineering. Woodhead Publishing, Philadelphia PA. 2012. Ch. 3, p46-47, the contents of which are herein incorporated by reference in their entirety).

As used herein, the term “light chain” refers to a component of an antibody from any vertebrate species assigned to one of two clearly distinct types, called kappa and lambda based on amino acid sequences of constant domains. Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2.

As used herein, the term “single chain Fv” or “scFv” refers to a fusion protein of V_{H} and V_{L}, antibody domains, wherein these domains are linked together into a single polypeptide chain by a flexible peptide linker. In some embodiments, the Fv polypeptide linker enables the scFv to form the desired structure for antigen binding. In some embodiments, scFvs are utilized in conjunction with phage display, yeast display or other display methods where they may be expressed in association with a surface member (e.g. phage coat protein) and used in the identification of high affinity peptides for a given antigen.

As used herein, the term “bispecific antibody” refers to an antibody capable of binding two different antigens. Such antibodies typically comprise regions from at least two different antibodies. Bispecific antibodies may include any of those described in Riethmuller, G. 2012. Cancer Immunity. 12:12-18, Marvin, J. S. et al., 2005. Acta Pharmacologica Sini ca. 26(6):649-58 and Schaefer, W. et al., 2011 PNAS. 108(27):11187-92, the contents of each of which are herein incorporated by reference in their entirety.

As used herein, the term “diabody” refers to a small antibody fragment with two antigen-binding sites. Diabodies comprise a heavy chain variable domain V_{H} connected to a light chain variable domain V_{L} in the same polypeptide chain. By using a linker that is too short to allow pairing between the two domains on the same chain, the domains are forced to pair with the complementary domains of another chain and create two antigen-binding sites. Diabodies are described more fully in, for example, EP 404097; WO 9311161; and Hollinger et al. (Hollinger, P. et al., “Diabodies”: Small bivalent and bispecific antibody fragments. PNAS. 1993. 90:6444-8) the contents of each of which are incorporated herein by reference in their entirety.

The term “intrabody” refers to a form of antibody that is not secreted from a cell in which it is produced, but instead targets one or more intracellular proteins. Intrabodies may be used to affect a multitude of cellular processes including, but not limited to intracellular trafficking, transcription, translation, metabolic processes, proliferative signaling, and cell division. In some embodiments, methods of the present disclosure may include intrabody-based therapies. In some such embodiments, variable domain sequences and/or CDR sequences disclosed herein may be incorporated into one or more constructs for intrabody-based therapy.

As used herein, the term “monoclonal antibody” refers to an antibody obtained from a population of substantially homogeneous cells (or clones), i.e., the individual antibodies comprising the population are identical and/or bind the same epitope, except for possible variants that may arise during production of the monoclonal antibodies, such variants generally being present in minor amounts. In contrast to polyclonal antibody preparations that typically include different antibodies directed against different determinants (epitopes), each monoclonal antibody is directed against a single determinant on the antigen

The modifier “monoclonal” indicates the character of the antibody as being obtained from a substantially homogeneous population of antibodies, and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

As used herein, the term “humanized antibody” refers to a chimeric antibody comprising a minimal portion from one or more non-human (e.g., murine) antibody source(s) with the remainder derived from one or more human immunoglobulin sources. For the most part, humanized antibodies are human immunoglobulins (recipient antibody) in which residues from the hypervariable region from an antibody of the recipient are replaced by residues from the hypervariable region from an antibody of a nonhuman species (donor antibody) such as mouse, rat, rabbit or nonhuman primate having the desired specificity, affinity, and/or capacity.

In some embodiments, viral genomes of the present disclosure may encode antibody mimetics. As used herein, the term “antibody mimetic” refers to any molecule which mimics the function or effect of an antibody and which binds specifically and with high affinity to their molecular targets. In some embodiments, antibody mimetics may be monobodies, designed to incorporate the fibronectin type III domain (Fn3) as a protein scaffold (U.S. Pat. Nos. 6,673,901; 6,348,584). In some embodiments, antibody mimetics may be those known in the art including, but are not limited to affibody molecules, affilins, affirms, anticalins, avimers, Centyrins, DARPINS™, fynomers, Kunitz domains, and domain peptides. In other embodiments, antibody mimetics may include one or more non-peptide regions.

As used herein, the term “antibody variant” refers to a modified antibody (in relation to a native or starting antibody) or a biomolecule resembling a native or starting antibody in structure and/or function (e.g., an antibody mimetic). Antibody variants may be altered in their amino acid sequence, composition, or structure as compared to a native antibody. Antibody variants may include, but are not limited to, antibodies with altered isotypes (e.g., IgA, IgD, IgC₁, IgG₂, IgG₃, IgG₄, or IgM); humanized variants, optimized variants, multispecific antibody variants (e.g., bispecific variants), and antibody fragments.

The preparation of antibodies, whether monoclonal or polyclonal. is known in the art. Techniques for the production of antibodies are well known in the art and described, e.g. in Harlow and Lane “Antibodies, A Laboratory Manual”, Cold Spring Harbor Laboratory Press, 1988; Harlow and Lane “Using Antibodies: A Laboratory Manual” Cold Spring Harbor Laboratory Press, 1999 and “Therapeutic Antibody Engineering: Current and Future Advances Driving the Strongest Growth Area in the Pharmaceutical Industry” Woodhead Publishing, 2012.

### Multispecific Antibodies

In some embodiments, payloads of the disclosure may encode antibodies that bind more than one epitope. As used herein, the terms “multibody” or “multispecific antibody” refer to an antibody wherein two or more variable regions bind to different epitopes. The epitopes may be on the same or different targets. In certain embodiments, a multi-specific antibody is a “bispecific antibody,” which recognizes two different epitopes on the same or different antigens.

In some embodiments, multi-specific antibodies may be prepared by the methods used by BIOATLA® and described in International Patent publication WO201109726; the contents of which are herein incorporated by reference in their entirety. First a library of homologous, naturally occurring antibodies is generated by any method known in the art (i.e., mammalian cell surface display), then screened by FACSAria or another screening method, for multi-specific antibodies that specifically bind to two or more target antigens. In some embodiments, the identified multi-specific antibodies are further evolved by any method known in the art, to produce a set of modified multi-specific antibodies. These modified multi-specific antibodies are screened for binding to the target antigens. In some embodiments, the multi-specific antibody may be further optimized by screening the evolved modified multi-specific antibodies for optimized or desired characteristics.

In some embodiments, multi-specific antibodies may be prepared by the methods used by BIOATLA® and described in Unites States Publication No. US20150252119, the contents of which are herein incorporated by reference in their entirety. In one approach, the variable domains of two parent antibodies, wherein the parent antibodies are monoclonal antibodies are evolved using any method known in the art in a manner that allows a single light chain to functionally complement heavy chains of two different parent antibodies. Another approach requires evolving the heavy chain of a single parent antibody to recognize a second target antigen. A third approach involves evolving the light chain of a parent antibody so as to recognize a second target antigen. Methods for polypeptide evolution are described in International Publication WO2012009026, the contents of which are herein incorporated by reference in their entirety, and include as non-limiting examples, Comprehensive Positional Evolution (CPE), Combinatorial Protein Synthesis (CPS), Comprehensive Positional Insertion (CH), Comprehensive Positional Deletion (CPD), or any combination thereof. The Fc region of the multi-specific antibodies described in United States Publication No. US20150252119 may be created using a knob-in-hole approach, or any other method that allows the Fc domain to form heterodimers. The resultant multi-specific antibodies may be further evolved for improved characteristics or properties such as binding affinity for the target antigen.

### Bispecific Antibodies

In some embodiments, payloads of the disclosure may encode bispecific antibodies. Bispecific antibodies are capable of binding two different antigens. Such antibodies typically comprise antigen-binding regions from at least two different antibodies. For example, a bispecific monoclonal antibody (BsMAb, BsAb) is an artificial protein composed of fragments of two different monoclonal antibodies, thus allowing the BsAb to bind to two different types of antigen.

In some cases, payloads encode bispecific antibodies comprising antigen-binding regions from two different antibodies. For example, such bi specific antibodies may comprise binding regions from two different antibodies selected from Tables 3-53.

Bispecific antibody frameworks may include any of those described in Riethmuller, G., 2012. Cancer Immunity. 12:12-18; Marvin, J. S. et al., 2005. Acta Pharmacologica Sinica. 26(6):649-58; and Schaefer, W. et at, 2011. PNAS. 108(27):11187-92, the contents of each of which are herein incorporated by reference in their entirety.

New generations of BsMAb, called “trifunctional bispecific” antibodies, have been developed. These consist of two heavy and two light chains, one each from two different antibodies, where the two Fab regions (the arms) are directed against two antigens, and the Fe region (the foot) comprises the two heavy chains and forms the third binding site.

Of the two paratopes that form the tops of the variable domains of a bispecific antibody, one can be directed against a target antigen and the other against a T-lymphocyte antigen like CD3. In the case of trifunctional antibodies, the Fc region may additionally bind to a cell that expresses Fc receptors, like a macrophage, a natural killer (NK) cell or a dendritic cell. In sum, the targeted cell is connected to one or two cells of the immune system, which subsequently destroy it.

Other types of bispecific antibodies have been designed to overcome certain problems, such as short half-life, immunogenicity and side-effects caused by cytokine liberation. They include chemically linked Fabs, consisting only of the Fab regions, and various types of bivalent and trivalent single-chain variable fragments (scFvs), fusion proteins mimicking the variable domains of two antibodies. The furthest developed of these newer formats are the bi-specific T-cell engagers (BiTEs) and mAb2′s, antibodies engineered to contain an Fcab antigen-binding fragment instead of the Fc constant region.

Using molecular genetics, two scFvs can be engineered in tandem into a single polypeptide, separated by a linker domain, called a “tandem scFv” (tascFv). TascFvs have been found to be poorly soluble and require refolding when produced in bacteria, or they may be manufactured in mammalian cell culture systems, which avoids refolding requirements but may result in poor yields. Construction of a tascFv with genes for two different scFvs yields a “bispecific single-chain variable fragments” (bis-scFvs). Only two tascFvs have been developed clinically by commercial firms; both are bispecific agents in active early phase development by Micromet for oncologic indications, and are described as “Bispecific T-cell Engagers (BiTE).” Blinatumomab is an anti-CD19/anti-CD3 bispecific tascFv that potentiates T-cell responses to B-cell non-Hodgkin lymphoma in Phase 2. MT110 is an anti-EP-CAM/anti-CD3 bispecific tascFv that potentiates T-cell responses to solid tumors in Phase 1. Bispecific, tetravalent “TandAbs” are also being researched by Affimed (Nelson, A. L., MAbs.2010. January-February; 2(1):77-83).

In some embodiments, payloads may encode antibodies comprising a single antigen-binding domain. These molecules are extremely small, with molecular weights approximately one-tenth of those observed for full-sized mAbs. Further antibodies may include “nanobodies” derived from the antigen-binding variable heavy chain regions (V_{HH}s) of heavy chain antibodies found in camels and llamas, which lack light chains (Nelson, A. L., MAbs.2010. January-February; 2(1):77-83).

Disclosed and claimed in PCT Publication WO2014144573 to Memorial Sloan-Kettering Cancer Center are multimerization technologies for making dimeric multi specific binding agents (e.g., fusion proteins comprising antibody components) with improved properties over multi specific binding agents without the capability of dimerization.

In some cases, payloads of the disclosure may encode tetravalent bispecific antibodies (TetBiAbs as disclosed and claimed in PCT Publication WO2014144357). TetBiAbs feature a second pair of Fab fragments with a second antigen specificity attached to the C-terminus of an antibody, thus providing a molecule that is bivalent for each of the two antigen specificities. The tetravalent antibody is produced by genetic engineering methods, by linking an antibody heavy chain covalently to a Fab light chain, which associates with its cognate, co-expressed Fab heavy chain.

In some aspects, payloads of the disclosure may encode biosynthetic antibodies as described in U.S. Pat. No. 5,091,513, the contents of which are herein incorporated by reference in their entirety. Such antibody may include one or more sequences of amino acids constituting a region which behaves as a biosynthetic antibody binding site (BABS). The sites comprise 1) non-covalently associated or disulfide bonded synthetic V_{H} and V_{L} dimers, 2) V_{H}-V_{L} or V_{L}-V_{H} single chains wherein the V_{H} and V_{L} are attached by a polypeptide linker, or 3) individuals V_{H} or V_{L} domains. The binding domains comprise linked CDR and FR regions, which may be derived from separate immunoglobulins. The biosynthetic antibodies may also include other polypeptide sequences which function, e.g., as an enzyme, toxin, binding site, or site of attachment to an immobilization media or radioactive atom. Methods are disclosed for producing the biosynthetic antibodies, for designing BAGS having any specificity that can be elicited by in vivo generation of antibody, and for producing analogs thereof.

In some embodiments, payloads may encode antibodies with antibody acceptor frameworks taught in U.S. Pat. No. 8,399,625. Such antibody acceptor frameworks may be particularly well suited accepting CDRs from an antibody of interest. In some cases, CDRs from anti-tau antibodies known in the art or developed according to the methods presented herein may be used.

### Miniaturized Antibody

In some embodiments, the antibody encoded by the payloads of the disclosure may be a “miniaturized” antibody. Among the best examples of mAb miniaturization are the small modular immunopharmaceuticals (SMIPs) from Trubion Pharmaceuticals. These molecules, which can be monovalent or bivalent, are recombinant single-chain molecules containing one V_{L}, one V_{H} antigen-binding domain, and one or two constant “effector” domains, all connected by linker domains. Presumably, such a molecule might offer the advantages of increased tissue or tumor penetration claimed by fragments while retaining the immune effector functions conferred by constant domains. At least three “miniaturized” SMIPs have entered clinical development. TRU-015, an anti-CD20 SMIP developed in collaboration with Wyeth, is the most advanced project, having progressed to Phase 2 for rheumatoid arthritis (RA). Earlier attempts in systemic lupus erythrematosus (SLE) and B cell lymphomas were ultimately discontinued. Trubion and Facet Biotechnology are collaborating in the development of TRU-016, an anti-CD37 SMIP, for the treatment of CLL and other lymphoid neoplasias, a project that has reached Phase 2. Wyeth has licensed the anti-CD20 SMTP SBI-087 for the treatment of autoimmune diseases, including RA, SLE, and possibly multiple sclerosis, although these projects remain in the earliest stages of clinical testing. (Nelson, A. L., MAbs.2010. January-February; 2(1):77-83).

### Diabodies

In some embodiments, payloads of the disclosure may encode diabodies. Diabodies are functional bispecific single-chain antibodies (bscAb). These bivalent antigen-binding molecules are composed of non-covalent dimers of scFvs, and can be produced in mammalian cells using recombinant methods. (See, e.g., Mack et al. Proc. Natl. Acad. Sci., 92: 7021-7025, 1995). Few diabodies have entered clinical development. An iodine-123-labeled diabody version of the anti-CEA chimeric antibody cT84.66 has been evaluated for pre-surgical immunoscintigraphic detection of colorectal cancer in a study sponsored by the Beckman Research Institute of the City of Hope (Clinicaltrials.gov NCT00647153) (Nelson, A. L., MAbs., 2010. January-February; 2(1):77-83),

### Unibody

In some embodiments, payloads may encode a “unibody,” in which the hinge region has been removed from IgG4 molecules. While IgG4 molecules are unstable and can exchange light-heavy chain heterodimers with one another, deletion of the hinge region prevents heavy chain-heavy chain pairing entirely, leaving highly specific monovalent light/heavy heterodimers, while retaining the Fe region to ensure stability and half-life in vivo. This configuration may minimize the risk of immune activation or oncogenic growth, as IgG4 interacts poorly with FcRs and monovalent unibodies fail to promote intracellular signaling complex formation. These contentions are, however, largely supported by laboratory, rather than clinical. evidence. Other antibodies may be “miniaturized” antibodies, which are compacted 100 kDa antibodies (see, e.g., Nelson, A. L., MAbs., 2010. January-February; 2(1):77-83).

### Intrabodies

In some embodiments, payloads of the disclosure may encode intrabodies. Intrabodies are a form of antibody that is not secreted from a cell in which it is produced, but instead targets one or more intracellular proteins. Intrabodies are expressed and function intracellularly and may be used to affect a multitude of cellular processes including, but not limited to intracellular trafficking, transcription, translation, metabolic processes, proliferative signaling and cell division. In some embodiments, methods described herein include intrabody-based therapies. In some such embodiments, variable domain sequences and/or CDR sequences disclosed herein are incorporated into one or more constructs for intrabody based therapy. For example, intrabodies may target one or more glycated intracellular proteins or may modulate the interaction between one or more glycated intracellular proteins and an alternative protein.

More than two decades ago, intracellular antibodies against intracellular targets were first described (Biocca, Neuberger and Cattaneo EMBO J. 9: 101-108, 1990). The intracellular expression of intrabodies in different compartments of mammalian cells allows blocking or modulation of the function of endogenous molecules (Biocca, et al., EMBO J. 9: 101-108, 1990; Colby et al., Proc. Natl. Acad. Sci. U.S.A. 101: 17616-21, 2004). Intrabodies can alter protein folding, protein-protein, protein-DNA, protein-RNA interactions and protein modification. They can induce a phenotypic knockout and work as neutralizing agents by direct binding to the target antigen, by diverting its intracellular trafficking or by inhibiting its association with binding partners. They have been largely employed as research tools and are emerging as therapeutic molecules for the treatment of human diseases such as viral pathologies, cancer and misfolding diseases. The fast-growing bio-market of recombinant antibodies provides intrabodies with enhanced binding specificity, stability, and solubility, together with lower immunogenicity, for their use in therapy (Biocca, abstract in Antibody Expression and Production Cell Engineering Volume 7, 2011, pp. 179-195).

In some embodiments, intrabodies have advantages over interfering RNA (iRNA); for example, iRNA has been shown to exert multiple nonspecific effects, whereas intrabodies have been shown to have high specificity and affinity to target antigens. Furthermore, as proteins, intrabodies possess a much longer active half-life than iRNA. Thus, when the active half-life of the intracellular target molecule is long, gene silencing through iRNA may be slow to yield an effect, whereas the effects of intrabody expression can be almost instantaneous. Lastly, it is possible to design intrabodies to block certain binding interactions of a particular target molecule, while sparing others.

Intrabodies are often single chain variable fragments (scFvs) expressed from a recombinant nucleic acid molecule and engineered to be retained intracellularly (e.g., retained in the cytoplasm, endoplasmic reticulum, or periplasm). Intrabodies may be used, for example, to ablate the function of a protein to which the intrabody binds. The expression of intrabodies may also be regulated through the use of inducible promoters in the nucleic acid expression vector comprising the intrabody. Intrabodies may be produced for use in the viral genomes of the disclosure using methods known in the art, such as those disclosed and reviewed in: (Marasco et al. 1993 Proc. Natl. Acad. Sci. USA, 90: 7889-7893; Chen et al., 1994, Hum. Gene Tiler. 5:595-601; Chen et al., 1994, Proc. Natl. Acad. Sci. USA, 91: 5932-5936; Maciejewski et at, 1995, Nature Med., 1: 667-673; Marasco, 1995, Immunotech, 1: 1-19; Mhashilkar, et al., 1995, EMBO J. 14: 1542-51; Chen et al., 1996, Hum. Gene Therap., 7: 1515-1525; Marasco, Gene They 4:11-15, 1997; Rondon and Marasco, 1997, Annu. Rev. Microbial. 51:257-283; Cohen, et al., 1998, Oncogene 17:2445-56; Proba et a., 1998, Mol. Biol. 275:245-253; Cohen et a., 1998, Oncogene 17:2445-2456; Hassanzadeh, et al., 1998, FEBS Lett. 437:81-6; Richardson et al., 1998, Gene Ther. 5:635-44; Ohage and Steipe, 1999, J. Mol. Biol. 291:1119-1128; Ohage et al., 1999, 1. Mot. Biol. 291:1129-1134; Wirtz and Steipe, 1999, Protein Sci. 8:2245-2250; Zhu et al., 1999, J. Immunol. Methods 231:207-222; Arafat et al., 2000, Cancer Gene Ther. 7:1250-6; der Maur et al., 2002, J Biol. Chem. 277:45075-85; Mhashilkar et al., 2002, Gene They 9:307-19; and Wheeler et al., 2003, FASEB J. 17: 1733-5; and references cited therein). In particular, a CCR5 intrabody has been produced by Steinberger et al., 2000, Proc. Natl. Acad. Sci. USA 97:805-810). See generally Marasco, W A, 1998, “Intrabodies: Basic Research and Clinical Gene Therapy Applications” Springer: New York; and for a review of scFvs, see Pluckthun in “The Pharmacology of Monoclonal Antibodies,” 1994, vol. 113, Rosenburg and Moore eds. Springer-Verlag, New York, pp. 269-315.

Sequences from donor antibodies may be used to develop intrabodies. Intrabodies are often recombinantly expressed as single domain fragments such as isolated V_{H} and V_{L} domains or as a single chain variable fragment (scFv) antibody within the cell. For example, intrabodies are often expressed as a single polypeptide to form a single chain antibody comprising the variable domains of the heavy and light chains joined by a flexible linker polypeptide. Intrabodies typically lack disulfide bonds and are capable of modulating the expression or activity of target genes through their specific binding activity. Single chain antibodies can also be expressed as a single chain variable region fragment joined to the light chain constant region.

As is known in the art, an intrabody can be engineered into recombinant polynucleotide vectors to encode sub-cellular trafficking signals at its N or C terminus to allow expression at high concentrations in the sub-cellular compartments where a target protein is located. For example, intrabodies targeted to the endoplasmic reticulum (ER) are engineered to incorporate a leader peptide and, optionally, a C-terminal ER retention signal. such as the KDEL amino acid motif (SEQ ID NO: 32691). Intrabodies intended to exert activity in the nucleus are engineered to include a nuclear localization signal. Lipid moieties are joined to intrabodies in order to tether the intrabody to the cytosolic side of the plasma membrane. Intrabodies can also be targeted to exert function in the cytosol. For example, cytosolic intrabodies are used to sequester factors within the cytosol, thereby preventing them from being transported to their natural cellular destination.

There are certain technical challenges with intrabody expression. In particular, protein conformational folding and structural stability of the newly-synthesized intrabody within the cell is affected by reducing conditions of the intracellular environment.

Intrabodies of the disclosure may be promising therapeutic agents for the treatment of misfolding diseases, including Tauopathies, prion diseases, Alzheimer's, Parkinson's, and Huntington's, because of their virtually infinite ability to specifically recognize the different conformations of a protein, including pathological isoforms, and because they can be targeted to the potential sites of aggregation (both intra and extracellular sites). These molecules can work as neutralizing agents against amyloidogenic proteins by preventing their aggregation, and/or as molecular shunters of intracellular traffic by rerouting the protein from its potential aggregation site (Cardinale, and Biocca, Curr. Mol. Med. 2008, 8:2-11).

### Maxibodies

In some embodiments, the payloads of the disclosure encode a maxibody (bivalent scFV fused to the amino terminus of the Fc (CH2-CH3 domains) of IgG.

### Chimeric Antigen Receptors

In some embodiments, the polypeptides encoded by the viral genomes of the disclosure (e.g., antibodies) may be used to generate chimeric antigen receptors (CARS) as described by BIOATLA® in International Publications WO2016033331 and WO2016036916, the contents of which are herein incorporated by reference in their entirety. As used herein, a “chimeric antigen receptor (CAR)” refers to an artificial chimeric protein comprising at least one antigen specific targeting region (ASTR), wherein the antigen specific targeting region comprises a full-length antibody or a fragment thereof that specifically binds to a target antigen. The ASTR may comprise any of the following; a full length heavy or light chain, an Fab fragment, a single chain Fv fragment, a divalent single chain antibody, or a diabody. As a non-limiting example the ASTR of a CAR may be any of the antibodies listed in Tables 3-53, antibody-based compositions or fragments thereof. Any molecule that is capable of binding a target antigen with high affinity can be used in the ASTR of a CAR. In some embodiments, the CAR may have more than one ASTR. These ASTRs may target two or more antigens or two or more epitopes of the same antigen. In some embodiments, the CAR is conditionally active. In some embodiments, the CAR is used to produce a genetically engineered cytotoxic cell carrying the CAR and capable of targeting the antigen bound by the ASTR.

Chimeric antigen receptors (CARO are particularly useful in the treatment of cancers, though also therapeutically effective in treatment of a wide variety of other diseases and disorders. Non-limiting examples of disease categories that may be treated with CARs or CAR-based therapeutics include autoimmune disorders, B-cell mediated diseases, inflammatory diseases, neuronal disorders, cardiovascular disease and circulatory disorders, or infectious diseases. Not wishing to be bound by theory, CARs traditionally work by targeting antigens presented on the surface of or on the inside of cells to be destroyed e.g., cancer tumor cells, by the cytotoxic cell of the CAR.

### Senescent Cell Surface Protein Antibodies

In some embodiments, the viral particles may comprise nucleic acids which have been engineered to express of antibodies that selectively bind to surface marker proteins of senescent cells. For example, the antibodies may selectively bind to proteins that are in misfolded conformation. The binding antibodies may reduce the number of senescent cells and be used to treat age-related conditions, such as, but not limited to, Alzheimer's disease, cardiovascular disease, emphysema, sarcopenia, and tumorigenesis as well as conditions more cosmetic in nature such as signs of skin aging including wrinkling, sagging, discoloration, age-related tissue dysfunction, tumor formation, and other age-related conditions.

In some embodiments, the expressed antibodies binding to epitopes of senescent cell surface proteins may be, but are not limited to, such as prion epitopes presented by SEQ ID NO: 1-14 of International Publication No. WO02014186878; CD44 epitopes presented by SEQ ID NO: 47-51 of International Publication No. WO2014186878; TNFR epitopes presented by SEQ ID NO: 52-56 of International Publication No. WO2014186878; NOTCH1 epitope presented by SEQ ID NO: 57-61 of International Publication No. WO2014186878; FasR epitopes presented by SEQ ID NO: 62-66 of International Publication No. WO2014186878; epidermal growth factor epitopes presented by SEQ ID NO: 67-81 of International Publication No. WO2014186878; CD38 epitopes presented by SEQ ID NO: 82-86 of International Publication No. WO2014186878, the contents of each of which are herein incorporated by reference in their entirety.

In some embodiments, the expressed antibodies may comprise peptides binding to senescent cell surface prion proteins, such as, but not limited to, those presented by SEQ ID NO: 15-36 of International Publication No. WO2014186878, the contents of which are herein incorporated by reference in their entirety.

In some embodiments, the expressed antibody may be AMF-3a-118 or AIF 3d-19 (SEQ ID NO: 8992 and 103106 of International publication WO2014186878, respectively, the contents of which are herein incorporated by reference in their entirety) targeting senescent cell surface protein FasR. In some embodiments, the expressed antibody may be Ab c-120 (SEQ NO: 37-40 of International publication WO2014186878, the contents of which are herein incorporated by reference in their entirety) targeting senescent cell surface protein PrP.

### Payload Antibodies of the Disclosure

In some embodiments, the payload region of the viral particle comprises one or more nucleic acid sequences encoding antibodies, variants or fragments thereof.

In some embodiments, the payload region of the viral particle comprises one or more nucleic acid sequences encoding TRIM21, variants or fragments thereof.

In some embodiments, the payload region of the viral particle comprises one or more nucleic acid sequences encoding antibody and TRIM21, variants or fragments thereof.

In some embodiments, the payload region of the viral particle comprises one or more nucleic acid sequences encoding one or more of the payload antibody polypeptides listed in any of International Publications, WO2017191559, WO2017191561 or WO2017191560 all to Prothena Biosciences, Limited, the contents of each of which are incorporated by reference herein in their entirety.

In some embodiments, the payload region of the viral particle comprises one or more nucleic acid sequences encoding one or more of the payload antibody polypeptides listed in Tables 3-53, or variants or fragments thereof. As used herein, “antibody polynucleotide” refers to a nucleic acid sequence encoding an antibody polypeptide.

In some embodiments, the payload region of the viral particle comprises one or more nucleic acid sequences listed in Tables 3-53, or variants or fragments thereof.

In some embodiments, the payload region of the viral particle comprises one or more nucleic acid sequences listed in Tables 3-53, or variants or fragments thereof, which result in production of a bispecific antibody. In some embodiments, the payload may be a bispecific antibody. The bispecific antibody may comprise one or more antibody components described herein or otherwise known in the art.

In some embodiments, the payload region of the viral particle comprises an Fe swap component, wherein said Fc swap may mediate direct cell killing. In some embodiments, the Fe swap component is introduced into a bispecific antibody payload.

In some embodiments, the payload region of the viral particle comprises a nucleic acid sequence encoding a payload antibody with at least 50% identity to one or more payload antibody polypeptides listed in Tables 3-53. The encoded antibody polypeptide may have 50%, 51%, 52%, 53%, 54%, 55%, 56%, 57%, 58%, 59%, 60%, 61%, 62%, 63%, 64%, 65%, 66%, 67% 68% 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 9398O, 94%, 95%, 96%, 97%, 98%, 99%, or 100% identity to one or more of the payload antibody polypeptides listed in Tables 3-53, or variants or fragments thereof.

In some embodiments, the full sequence of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 77%, 73%, 74%, 75%, 76%, 77%, 78%. 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Tables 3-53, or variants or fragments thereof.

In some embodiments, the variable region sequence(s) of the encoded antibody polypeptide may have 50%, 51%, 52%, 53%, 54%, 55%, 56%, s7%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 999/O, or 100% identity to one or more of the payload antibody polypeptides listed in Tables 3-53, or variants or fragments thereof.

In some embodiments, the heavy chain of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 811%, 82%, 83%, 84%, 85%, 86%, 87%, 88%, 89%, 90%, 91%, 92%, 93%, 94%, 95%, 96%, 97%, 98%, 99%, or 100% identity to one or more of the payload heavy chain antibody polypeptides listed in Tables 3-53, or variants or fragments thereof.

In some embodiments, the light chain of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87% 88%, 89%. 90%, 911%, 92%, 93%, 94%, 95%, 96%, 97%, 98%, 99%, or 100% identity to one or more of the payload light chain antibody polypeptides listed in Tables 3-53, or variants or fragments thereof.

In some embodiments, the CDR region of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%. 90%, 91%, 92%, 93%, 94%, 95%, 96%, 97%, 98%, 99%, or 100% identity to the CDRs of one or more of the payload antibody polypeptides listed in Tables 3-53, or variants or fragments thereof.

In some embodiments, the payload antibody has 90% identity to one or more of the antibody polypeptides listed in Tables 3-53, or variants or fragments thereof.

In some embodiments, the payload antibody has 91% identity to one or more of the antibody polypeptides listed in Tables 3-53, or variants or fragments thereof.

In some embodiments, the payload antibody has 92% identity to one or more of the antibody polypeptides listed in Tables 3-53, or variants or fragments thereof.

In some embodiments, the payload antibody has 93% identity to one or more of the antibody polypeptides listed in Tables 3-53, or variants or fragments thereof.

In some embodiments, the payload antibody has 94% identity to one or more of the antibody polypeptides listed in Tables 3-53, or variants or fragments thereof.

In some embodiments, the payload antibody has 95% identity to one or more of the antibody polypeptides listed in Tables 3-53, or variants or fragments thereof.

In some embodiments, the payload antibody has 96% identity to one or more of the antibody polypeptides listed in Tables 3-53, or variants or fragments thereof.

In some embodiments, the payload antibody has 97% identity to one or more of the antibody polypeptides listed in Tables 3-53, or variants or fragments thereof.

In some embodiments, the payload antibody has 98% identity to one or more of the antibody polypeptides listed in Tables 3-53, or variants or fragments thereof.

In some embodiments, the payload antibody has 99% identity to one or more of the antibody polypeptides listed in Tables 3-53, or variants or fragments thereof.

In some embodiments, the payload antibody has 100% identity to one or more of the antibody polypeptides listed in Tables 3-53, or variants or fragments thereof.

In some embodiments, the payload region of the viral particle comprises a nucleic acid sequence with at least 50% identity to one or more nucleic acid sequences listed in Tables 3-53, or variants or fragments thereof. The payload nucleic acid sequence may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 7700, 78%, 79%, 80%, 81%, 82′,O, 83%, 84%, 85%, 8600, 8700, 8800, 89%, 90%, 9100, 9200, 9300, 94% 9500, 9600, 97%, 9800, 9900 or 10000 identity to one or more nucleic acid sequences listed in Tables 3-53, or variants or fragments thereof.

In some embodiments, the payload nucleic acid sequence has 9000 identity to one or more of the nucleic acid sequences listed in Tables 3-53, or variants or fragments thereof.

In some embodiments, the payload nucleic acid sequence has 91% identity to one or more of the nucleic acid sequences listed in Tables 3-53, or variants or fragments thereof.

In some embodiments, the payload nucleic acid sequence has 92% identity to one or more of the nucleic acid sequences listed in Tables 3-53, or variants or fragments thereof.

In some embodiments, the payload nucleic acid sequence has 93% identity to one or more of the nucleic acid sequences listed in Tables 3-53, or variants or fragments thereof.

In some embodiments, the payload nucleic acid sequence has 94% identity to one or more of the nucleic acid sequences listed in Tables 3-53, or variants or fragments thereof.

In some embodiments, the payload nucleic acid sequence has 95% identity to one or more of the nucleic acid sequences listed in Tables 3-53, or variants or fragments thereof.

In some embodiments, the payload nucleic acid sequence has 96% identity to one or more of the nucleic acid sequences listed in Tables 3-53, or variants or fragments thereof.

In some embodiments, the payload nucleic acid sequence has 97% identity to one or more of the nucleic acid sequences listed in Tables 3-53, or variants or fragments thereof.

In some embodiments, the payload nucleic acid sequence has 98% identity to one or more of the nucleic acid sequences listed in Tables 3-53, or variants or fragments thereof.

In some embodiments, the payload nucleic acid sequence has 99% identity to one or more of the nucleic acid sequences listed in Tables 3-53, or variants or fragments thereof.

In some embodiments, the payload nucleic acid sequence has 100% identity to one or more of the nucleic acid sequences listed in Tables 3-53, or variants or fragments thereof.

In some embodiments, the payload region of the viral particle comprises a nucleic acid sequence encoding a polypeptide which is an antibody, an antibody-based composition, or a fragment thereof. As a non-limiting example, the antibody may be one or more of the polypeptides listed in Tables 3-53, or variants or fragments thereof. As another non-limiting example, the antibody may be one or more of the heavy chain sequences listed in Tables 3-53. As a non-limiting example, the antibody may be one or more of the light chain sequences listed in Tables 3-53, or variants or fragments thereof.

In some embodiments, the payload region of the viral particle comprises a nucleic acid sequence encoding a polypeptide comprising a heavy chain and a light chain sequence listed in Tables 3-53, or variants or fragments thereof. The payload region may also comprise a linker between the heavy and light chain sequences. The linker may be a sequence known in the art or described in Table 2.

In some embodiments, the payload region of the viral particle comprises a nucleic acid sequence encoding a polypeptide comprising a heavy chain and a light chain sequence listed in Tables 3-53, or variants or fragments thereof, where the heavy chain sequence is from a different antibody than the light chain sequence. The payload region may also comprise a linker between the heavy and light chain sequences. The linker may be a sequence known in the art or described in Table 2.

In some embodiments, the payload region comprises, in the 5′ to 3′ direction, an antibody light chain sequence, a linker and a heavy chain sequence.

In some embodiments, the payload region comprises a nucleic acid sequence encoding, in the 5′ to 3′ direction, an antibody light chain sequence from Tables 3-53, a linker from Table 2 and a heavy chain sequence from Tables 3-53.

In some embodiments, the payload region comprises, in the 5′ to 3′ direction, an antibody heavy chain sequence, a linker and a light chain sequence.

In some embodiments, the payload region comprises a nucleic acid sequence encoding, in the 5′ to 3′ direction, an antibody heavy chain sequence from Tables 3-53, a linker from Table 2, and a light chain sequence from Tables 3-53.

In some embodiments, the payload region comprises a nucleic acid sequence encoding a single heavy chain. As a non-limiting example, the heavy chain is an amino acid sequence or fragment thereof described in Tables 3-53.

Tables 3-53 provide a listing of antibodies and their polynucleotides and/or polypeptides sequences. These sequences may be encoded by or included in the viral particles of the present disclosure. Variants or fragments of the antibody sequences described in Tables 3-53 may be utilized in the viral particles of the present disclosure.

In some embodiments, the viral particles may comprise codon-optimized versions of the nucleic acids encoding the polypeptides listed in Tables 3-53. In some cases, the payload region of the viral particles of the disclosure may encode one or more isoforms or variants of these heavy and light chain antibody domains. Such variants may be humanized or optimized antibody domains comprising one or more complementarity determining regions (CDRs) from the heavy and light chains listed in Tables 3-53. CDRs of the antibodies encoded by the viral genomes of the present disclosure may be 50%, 60%, 70%, 80%, 90%, 95% identical to CDRs listed in or incorporated in the sequences of Tables 3-53. Methods of determining CDRs are well known in the art and are described herein. Payload regions may encode antibody variants with one or more heavy chain variable domain (V_{H}) or light chain variable domain (V_{L}) derived from the antibody sequences in Tables 3-53. In some cases, such variants may include bispecific antibodies. Bispecific antibodies encoded by payload regions of the disclosure may comprise variable domain pairs from two different antibodies.

In some embodiments, the viral particles may comprise a heavy and a light chain of an antibody described herein and two promoters. As a non-limiting example, the viral particles may comprise a nucleic acid sequence of a genome as described in FIG. 1 or FIG. 2 of US Patent Publication No. US20030219733, the contents of which are herein incorporated by reference in its entirety. As another non-limiting example, the viral particles may be a dual-promoter viral particle for antibody expression as described by Lewis et al. (J. of. Virology, September 2002, Vol. 76(17), p 8769-8775; the contents of which are herein incorporated by reference in its entirety).

Parkinson's Disease and Dementia with Lewy Bodies Antibodies

In some embodiments, the payload region of the viral particle comprises one or more nucleic acid sequences encoding one or more of the Parkinson's Disease and dementia with Lewy Bodies payload antibody polypeptides listed in Table 3 (PDLB1-PDLB437; SEQ ID NO: 3787-4223).

TABLE 3
Parkinson's Disease and Dementia with Lewy Bodies Antibodies

| Antibody No. | Target | Description | Antibody Name | Reference Information | SEQ ID NO |
|---|---|---|---|---|---|
| PDLB1 | amyloid proteins | consensus sequence | M13 g3p, fd g3p, fl g3p | US20150376239 | 3787 |
| | | | | SEQ ID NO: 4 | |
| PDLB2 | amyloid proteins | consensus sequence | I2-2 g3p, Ike g3p | US20150376239 | 3788 |
| | | | | SEQ ID NO: 7 | |
| PDLB3 | 118-126 of α- | constant region | IgG1 | US20150259404 | 3789 |
| | synuclein | | | SEQ ID NO: 38 | |
| PDLB4 | amyloid proteins | Fusion protein | M13 g3p | US20150376239 | 3790 |
| | | | | SEQ ID NO: 1 | |
| PDLB5 | amyloid proteins | Fusion protein | Construct 5 | US20150376239 | 3791 |
| | | | | SEQ ID NO: 11 | |
| PDLB6 | amyloid proteins | Fusion protein | Construct 6 | US20150376239 | 3792 |
| | | | | SEQ ID NO: 13 | |
| PDLB7 | amyloid proteins | Fusion protein | fd N2 | US20150376239 | 3793 |
| | | | | SEQ ID NO: 14 | |
| PDLB8 | amyloid proteins | Fusion protein | fl N2 | US20150376239 | 3794 |
| | | | | SEQ ID NO: 15 | |
| PDLB9 | amyloid proteins | Fusion protein | M13 N2 | US20150376239 | 3795 |
| | | | | SEQ ID NO: 16 | |
| PDLB10 | amyloid proteins | Fusion protein | Ike N2 | US20150376239 | 3796 |
| | | | | SEQ ID NO: 17 | |
| PDLB11 | amyloid proteins | Fusion protein | 12-2 N2 | US20150376239 | 3797 |
| | | | | SEQ ID NO: 18 | |
| PDLB12 | amyloid proteins | Fusion protein | If1 N2 | US20150376239 | 3798 |
| | | | | SEQ ID NO: 19 | |
| PDLB13 | amyloid proteins | Fusion protein | fd g3p | US20150376239 | 3799 |
| | | | | SEQ ID NO: 2 | |
| PDLB14 | amyloid proteins | Fusion protein | Construct 3 | US20150376239 | 3800 |
| | | | | SEQ ID NO: 20 | |
| PDLB15 | amyloid proteins | Fusion protein | Construct 3m g3p portion | US20150376239 | 3801 |
| | | | | SEQ ID NO: 24 | |
| PDLB16 | amyloid proteins | Fusion protein | If1 g3p | US20150376239 | 3802 |
| | | | | SEQ ID NO: 29 | |
| PDLB17 | amyloid proteins | Fusion protein | fl g3p | US20150376239 | 3803 |
| | | | | SEQ ID NO: 3 | |
| PDLB18 | amyloid proteins | Fusion protein | fd g3p | US20150376239 | 3804 |
| | | | | SEQ ID NO: 30 | |
| PDLB19 | amyloid proteins | Fusion protein | Construct 8, rs-g3p (If1- | US20150376239 | 3805 |
| | | | N1N2)-hlgG1-Fc | SEQ ID NO: 31 | |
| PDLB20 | amyloid proteins | Fusion protein | I2-2 g3p | US20150376239 | 3806 |
| | | | | SEQ ID NO: 5 | |
| PDLB21 | amyloid proteins | Fusion protein | Ike g3p | US20150376239 | 3807 |
| | | | | SEQ ID NO: 6 | |
| PDLB22 | amyloid proteins | Fusion protein | If1 g3p | US20150376239 | 3808 |
| | | | | SEQ ID NO: 8 | |
| PDLB23 | amyloid proteins | Fusion protein | Construct 4 | US20150376239 | 3809 |
| | | | | SEQ ID NO: 9 | |
| PDLB24 | 118-126 of α- | Heavy chain | 5ClH1 | US20150259404 | 3810 |
| | synuclein | | | SEQ ID NO: 14 | |
| PDLB25 | 118-126 of α- | Heavy chain | 5ClH2 | US20150259404 | 3811 |
| | synuclein | | | SEQ ID NO: 15 | |
| PDLB26 | 118-126 of α- | Heavy chain | 5ClH3 | US20150259404 | 3812 |
| | synuclein | | | SEQ ID NO: 16 | |
| PDLB27 | 118-126 of α- | Heavy chain | 5ClH4 | US20150259404 | 3813 |
| | synuclein | | | SEQ ID NO: 17 | |
| PDLB28 | 118-126 of α- | Heavy chain | 5ClH5 | US20150259404 | 3814 |
| | synuclein | | | SEQ ID NO: 18 | |
| PDLB29 | 118-126 of α- | Heavy chain | 5Cl | US20150259404 | 3815 |
| | synuclein | | | SEQ ID NO: 6 | |
| PDLB30 | ACTH | Heavy chain | Ab7 | WO2015127288 | 3816 |
| | | | | SEQ ID NO: 241 | |
| PDLB31 | ACTH | Heavy chain | Ab9 | WO2015127288 | 3817 |
| | | | | SEQ ID NO: 281 | |
| PDLB32 | ACTH | Heavy chain | Ab10 | WO2015127288 | 3818 |
| | | | | SEQ ID NO: 321 | |
| PDLB33 | ACTH | Heavy chain | Ab11 | WO2015127288 | 3819 |
| | | | | SEQ ID NO: 361 | |
| PDLB34 | ACTH | Heavy chain | Ab12 | WO2015127288 | 3820 |
| | | | | SEQ ID NO: 401 | |
| PDLB35 | ACTH | Heavy chain | Ab2 | WO2015127288 | 3821 |
| | | | | SEQ ID NO: 41 | |
| PDLB36 | ACTH | Heavy chain | Ab1.H | WO2015127288 | 3822 |
| | | | | SEQ ID NO: 441 | |
| PDLB37 | ACTH | Heavy chain | Ab2.H | WO2015127288 | 3823 |
| | | | | SEQ ID NO: 481 | |
| PDLB38 | ACTH | Heavy chain | Ab3.H | WO2015127288 | 3824 |
| | | | | SEQ ID NO: 521 | |
| PDLB39 | ACTH | Heavy chain | Ab4.H | WO2015127288 | 3825 |
| | | | | SEQ ID NO: 561 | |
| PDLB40 | ACTH | Heavy chain | Ab6.H | WO2015127288 | 3826 |
| | | | | SEQ ID NO: 601 | |
| PDLB41 | ACTH | Heavy chain | Ab7.H | WO2015127288 | 3827 |
| | | | | SEQ ID NO: 641 | |
| PDLB42 | ACTH | Heavy chain | Ab7A.H | WO2015127288 | 3828 |
| | | | | SEQ ID NO: 681 | |
| PDLB43 | ACTH | Heavy chain | Ab10.H | WO2015127288 | 3829 |
| | | | | SEQ ID NO: 721 | |
| PDLB44 | ACTH | Heavy chain | Ab11.H | WO2015127288 | 3830 |
| | | | | SEQ ID NO: 761 | |
| PDLB45 | ACTH | Heavy chain | Ab11A.H | WO2015127288 | 3831 |
| | | | | SEQ ID NO: 801 | |
| PDLB46 | ACTH | Heavy chain | Ab3 | WO2015127288 | 3832 |
| | | | | SEQ ID NO: 81 | |
| PDLB47 | ACTH | Heavy chain | Ab12.H | WO2015127288 | 3833 |
| | | | | SEQ ID NO: 841 | |
| PDLB48 | ACTH | Heavy chain | Ab4 | WO2015127288 | 3834 |
| | | | | SEQ ID NO: 121 | |
| PDLB49 | ACTH | Heavy chain | Ab5 | WO2015127288 | 3835 |
| | | | | SEQ ID NO: 161 | |
| PDLB50 | ACTH | Heavy chain | Ab6 | WO2015127288 | 3836 |
| | | | | SEQ ID NO: 201 | |
| PDLB51 | ACTH (Cushing's, | Heavy chain | Ab1 | WO2015127288 | 3837 |
| | PD, AD, anxiety | | | SEQ ID NO: 1 | |
| | disorders) | | | | |
| PDLB52 | alpha synuclein | Heavy chain | Hu1H7VHv1 | U.S. Pat. No. 8,790,644 | 3838 |
| | | | | SEQ ID NO: 19 | |
| PDLB53 | alpha synuclein | Heavy chain | Hu1H7VHv2 | U.S. Pat. No. 8,790,644 | 3839 |
| | | | | SEQ ID NO: 21 | |
| PDLB54 | alpha synuclein | Heavy chain | Hu1H7VHv3 | U.S. Pat. No. 8,790,644 | 3840 |
| | | | | SEQ ID NO: 23 | |
| PDLB55 | alpha synuclein | Heavy chain | Hu1H7VHv4 | U.S. Pat. No. 8,790,644 | 3841 |
| | | | | SEQ ID NO: 25 | |
| PDLB56 | alpha synuclein | Heavy chain | Hu1H7VHv5 | U.S. Pat. No. 8,790,644 | 3842 |
| | | | | SEQ ID NO: 27 | |
| PDLB57 | alpha synuclein | Heavy chain | Hu1H7VHv alternative | U.S. Pat. No. 8,790,644 | 3843 |
| | | | | SEQ ID NO: 44 | |
| PDLB58 | alpha synuclein | Heavy chain | Hu1H7VHv alternatives | U.S. Pat. No. 8,790,644 | 3844 |
| | | | | SEQ ID NO: 46 | |
| PDLB59 | alpha synuclein | Heavy chain | Humanized 5C 1H2 | WO2015075635 | 3845 |
| | | | | SEQ ID NO: 59 | |
| PDLB60 | alpha synuclein | Heavy chain | Humanized 5C 1H5 | WO2015075635 | 3846 |
| | | | | SEQ ID NO: 62 | |
| PDLB61 | alpha synuclein | Heavy chain | Hu1H7VH alternative | WO2015075635 | 3847 |
| | | | | SEQ ID NO: 121 | |
| PDLB62 | alpha synuclein | Heavy chain | Humanized 1 H7 heavy | WO2015075635 | 3848 |
| | | | chain version 3 (variable | SEQ ID NO: 126 | |
| | | | region + constant region) | | |
| PDLB63 | alpha synuclein | Heavy chain | Humanized 1H7 heavy | WO2015075635 | 3849 |
| | | | chain version 3 (variable | SEQ ID NO: 127 | |
| | | | region + constant region | | |
| | | | Gl m3 allotype) | | |
| PDLB64 | alpha synuclein | Heavy chain | Hu9E4VH alternative | WO2015075635 | 3850 |
| | | | | SEQ ID NO: 29 | |
| PDLB65 | alpha synuclein | Heavy chain | Humanized 9E4 heavy | WO2015075635 | 3851 |
| | | | chain version 3 (variable | SEQ ID NO: 34 | |
| | | | region + constant region) | | |
| PDLB66 | alpha synuclein | Heavy chain | Humanized 9E4 heavy | WO2015075635 | 3852 |
| | | | chain version 3 (variable | SEQ ID NO: 36 | |
| | | | region + constant region) | | |
| PDLB67 | alpha synuclein | Heavy chain | Humanized 9E4 heavy | WO2015075635 | 3853 |
| | | | chain version 3 (variable | SEQ ID NO: 37 | |
| | | | region + alternative | | |
| | | | constant region Glm3 | | |
| | | | allotype) | | |
| PDLB68 | alpha synuclein | Heavy chain | Humanized 5C 1 H1 | WO2015075635 | 3854 |
| | | | | SEQ ID NO: 58 | |
| PDLB69 | alpha synuclein | Heavy chain | Humanized 5C 1H3 | WO2015075635 | 3855 |
| | | | | SEQ ID NO: 60 | |
| PDLB70 | alpha synuclein | Heavy chain | Humanized 5C 1H4 | WO2015075635 | 3856 |
| | | | | SEQ ID NO: 61 | |
| PDLB71 | amyloids | Heavy chain | #118 | WO2010012004 | 3857 |
| | | | | SEQ ID NO: 11 | |
| PDLB72 | amyloids | Heavy chain | #121 | WO2010012004 | 3858 |
| | | | | SEQ ID NO: 13 | |
| PDLB73 | amyloids | Heavy chain | #204 | WO2010012004 | 3859 |
| | | | | SEQ ID NO: 16 | |
| PDLB74 | amyloids | Heavy chain | #205 | WO2010012004 | 3860 |
| | | | | SEQ ID NO: 18 | |
| PDLB75 | EAG1 | Heavy chain | chimeric ImAb3 | WO2006037604 | 3861 |
| | | | | SEQ ID NO: 12 | |
| PDLB76 | EAG1 | Heavy chain | chimeric ImAb4 | WO2006037604 | 3862 |
| | | | | SEQ ID NO: 16 | |
| PDLB77 | EAG1 | Heavy chain | HC-lmAb3-humVH3-72 | WO2006037604 | 3863 |
| | | | | SEQ ID NO: 20 | |
| PDLB78 | EAG1 | Heavy chain | HC-lmAb4-humVH4-59 | WO2006037604 | 3864 |
| | | | | SEQ ID NO: 24 | |
| PDLB79 | EAG1 | Heavy chain | HC-lmAb3-humVH3 23 | WO2006037604 | 3865 |
| | | | | SEQ ID NO: 28 | |
| PDLB80 | EAG1 | Heavy chain | HC-lmAb3-humVH2 26 | WO2006037604 | 3866 |
| | | | | SEQ ID NO: 32 | |
| PDLB81 | EAG1 | Heavy chain | HC-lmAb4-humVH1-3 | WO2006037604 | 3867 |
| | | | | SEQ ID NO: 36 | |
| PDLB82 | EAG1 | Heavy chain | ImAb4 | WO2006037604 | 3868 |
| | | | | SEQ ID NO: 4 | |
| PDLB83 | EAG1 | Heavy chain | ImAb3 | WO2006037604 | 3869 |
| | | | | SEQ ID NO: 8 | |
| PDLB84 | NOGO | Heavy chain | H6L13 FL | US20140147435 | 3870 |
| | | | | SEQ ID NO: 27 | |
| PDLB85 | NOGO | Heavy chain | H16L16 FL, H16L18 FL | US20140147435 | 387 |
| | | | | SEQ ID NO: 31 | |
| PDLB86 | NOGO | Heavy chain | H18L16 FL | US20140147435 | 3872 |
| | | | | SEQ ID NO: 33 | |
| PDLB87 | NOGO | Heavy chain | H19L13 FL, H19L16 FL, | US20140147435 | 3873 |
| | | | H19L18 FL | SEQ ID NO: 92 | |
| PDLB88 | NOGO | Heavy chain | H20L13 FL, H20L16 FL, | US20140147435 | 3874 |
| | | | H20L18 FL | SEQ ID NO: 93 | |
| PDLB89 | NOGO | Heavy chain | H21L13 FL, H21L16 FL, | US20140147435 | 3875 |
| | | | H21L18 FL | SEQ ID NO: 94 | |
| PDLB90 | NOGO | Heavy chain | H25L13 FL, H25L16 FL, | US20140147435 | 3876 |
| | | | H25L18 FL | SEQ ID NO: 98 | |
| PDLB91 | Nogo receptor-1 | Heavy chain | 5B10 | US20090215691 | 3877 |
| | | | | SEQ ID NO: 16 | |
| PDLB92 | Nogo receptor-1 | Heavy chain | 5B10 | US20090215691 | 3878 |
| | | | | SEQ ID NO: 18 | |
| PDLB93 | trk-C (NT-3 trkC | Heavy chain | 2250 | U.S. Pat. No. 7,615,383 | 3879 |
| | ligand) | | | SEQ ID NO: 42 | |
| PDLB94 | trk-C (NT-3 trkC | Heavy chain | 2253 | U.S. Pat. No. 7,615,383 | 3880 |
| | ligand) | | | SEQ ID NO: 43 | |
| PDLB95 | trk-C (NT-3 trkC | Heavy chain | 2256 | U.S. Pat. No. 7,615,383 | 3881 |
| | ligand) | | | SEQ ID NO: 44 | |
| PDLB96 | trk-C (NT-3 trkC | Heavy chain | 6.1.2 | U.S. Pat. No. 7,615,383 | 3882 |
| | ligand) | | | SEQ ID NO: 45 | |
| PDLB97 | trk-C (NT-3 trkC | Heavy chain | 6.4.1 | U.S. Pat. No. 7,615,383 | 3883 |
| | ligand) | | | SEQ ID NO: 46 | |
| PDLB98 | trk-C (NT-3 trkC | Heavy chain | 2345 | U.S. Pat. No. 7,615,383 | 3884 |
| | ligand) | | | SEQ ID NO: 47 | |
| PDLB99 | trk-C (NT-3 trkC | Heavy chain | 2349 | U.S. Pat. No. 7,615,383 | 3885 |
| | ligand) | | | SEQ ID NO: 48 | |
| PDLB100 | alpha synuclein | Heavy chain | Hu9E4VH consensus | U.S. Pat. No. 8,609,820 | 3886 |
| | | consensus chain | amino acid sequence | SEQ ID NO: 27 | |
| PDLB101 | alpha synuclein | Heavy chain | m9E4VH | WO2015075635 | 3887 |
| | | consensus chain | | SEQ ID NO: 6 | |
| PDLB102 | alpha synuclein | Heavy chain | Humanized 1H7 heavy | WO2015075635 | 3888 |
| | | constant region | chain constant region | SEQ ID NO: 128 | |
| | | | (IgG2) | | |
| PDLB103 | alpha synuclein | Heavy chain | Humanized 1H7 heavy | WO2015075635 | 3889 |
| | | constant region | chain constant region | SEQ ID NO: 129 | |
| | | | (Glm1 allotype) | | |
| PDLB104 | alpha synuclein | Heavy chain | Humanized 9E4 heavy | WO2015075635 | 3890 |
| | | constant region | chain constant region | SEQ ID NO: 35 | |
| | | | (Glm3 allotype: BIP | | |
| | | | version) | | |
| PDLB105 | alpha synuclein | Heavy chain | Hu1H7 | U.S. Pat. No. 8,790,644 | 3891 |
| | | constant region | | SEQ ID NO: 58 | |
| | | (G1m1 allotype) | | | |
| PDLB106 | alpha syrinclein | Heavy chain | Hu1H7 | U.S. Pat. No. 8,790,644 | 3892 |
| | | constant region | | SEQ ID NO: 52 | |
| | | G1m3 allotype) | | | |
| PDLB107 | alpha synuclein | Heavy chain | Hu1H7 | U.S. Pat. No. 8,790,644 | 3893 |
| | | constant region | | SEQ ID NO: 50 | |
| | | (IgG1; common for | | | |
| | | v1-v5) | | | |
| PDLB108 | alpha synuclein | Heavy chain | Hu1H7 | U.S. Pat. No. 8,790,644 | 3894 |
| | | constant region | | SEQ ID NO: 57 | |
| | | (IgG2) | | | |
| PDLB109 | many - growth | Heavy chain fusion | H19L13, H19L16, | U.S. Pat. No. 8,053,569 | 3895 |
| | factors (to | protein | H19L18, H19L14, | SEQ ID NO: 25 | |
| | increase transport | | H19L15, H19L17, H19L6, | | |
| | across BBB) | | H19L11 | | |
| PDLB110 | many - growth | Heavy chain fusion | H20L13, H20L16, | U.S. Pat. No. 8,053,569 | 3896 |
| | factors (to | protein | H20L18, H20L14, | SEQ ID NO: 28 | |
| | increase transport | | H20L15, H20L17, H2016, | | |
| | across BBB) | | H20L11 | | |
| PDLB111 | many - growth | Heavy chain fusion | H22L13, H22L16, | U.S. Pat. No. 8,053,569 | 3897 |
| | factors (to | protein | H22L18, H22L14, | SEQ ID NO: 34 | |
| | increase transport | | H22L15, H22L17, H22L6, | | |
| | across BBB) | | H22L11 | | |
| PDLB112 | many - growth | Heavy chain fusion | H5L11, H6L11, H14L11, | U.S. Pat. No. 8,053,569 | 3898 |
| | factors (to | protein | H15L11, H16L11, | SEQ ID NO: 24 | |
| | increase transport | | H17L11, H18L11, | | |
| | across BRB) | | H19L11, H20L11, | | |
| | | | H21L11, H22L11, | | |
| | | | H23L11, H24L11, | | |
| | | | H25L11, H700L11 | | |
| PDLB113 | NOGO | Heavy chain | 2A10 construct | WO2007003421 | 3899 |
| | | humanized construct | | SEQ ID NO: 79 | |
| | | H1 | | | |
| PDLB114 | NOGO | Heavy chain | 2A10 construct | WO2007003421 | 3900 |
| | | humanized construct | | SEQ ID NO: 29 | |
| | | H14 | | | |
| PDLB115 | NOGO | Heavy chain | 2A10 construct | WO2007003421 | 3901 |
| | | humanized construct | | SEQ ID NO: 30 | |
| | | H15 | | | |
| PDLB116 | NOGO | Heavy chain | 2A10 construct | WO2007003421 | 3902 |
| | | humanized construct | | SEQ ID NO: 31 | |
| | | H16 | | | |
| PDLB117 | NOGO | Heavy chain | 2A10 construct | WO2007003421 | 3903 |
| | | humanized construct | | SEQ ID NO: 32 | |
| | | H17 | | | |
| PDLB118 | NOGO | Heavy chain | 2A10 construct | WO2007003421 | 3904 |
| | | humanized construct | | SEQ ID NO: 33 | |
| | | H18 | | | |
| PDLB119 | NOGO | Heavy chain | 2A10 construct | WO2007003421 | 3905 |
| | | humanized construct | | SEQ ID NO: 92 | |
| | | H19 | | | |
| PDLB120 | NOGO | Heavy chain | 2A10 construct | WO2007003421 | 3906 |
| | | humanized construct | | SEQ ID NO: 93 | |
| | | H20 | | | |
| PDLB121 | NOGO | Heavy chain | 2A10 construct | WO2007003421 | 3907 |
| | | humanized construct | | SEQ ID NO: 94 | |
| | | H21 | | | |
| PDLB122 | NOGO | Heavy chain | 2A10 construct | WO2007003421 | 3908 |
| | | humanized construct | | SEQ ID NO: 95 | |
| | | H22 | | | |
| PDLB123 | NOGO | Heavy chain | 2A10 construct | WO2007003421 | 3909 |
| | | humanized construct | | SEQ ID NO: 96 | |
| | | H23 | | | |
| PDLB124 | NOGO | Heavy chain | 2A10 construct | WO2007003421 | 3910 |
| | | humanized construct | | SEQ ID NO: 97 | |
| | | H24 | | | |
| PDLB125 | NOGO | Heavy chain | 2A10 construct | WO2007003421 | 3911 |
| | | humanized construct | | SEQ ID NO: 98 | |
| | | H25 | | | |
| PDLB126 | NOGO | Heavy chain | 2A10 construct | WO2007003421 | 3912 |
| | | humanized construct | | SEQ ID NO: 26 | |
| | | H5 | | | |
| PDLB127 | NOGO | Heavy chain | 2A10 construct | WO2007003421 | 3913 |
| | | humanized construct | | SEQ ID NO: 27 | |
| | | H6 | | | |
| PDLB128 | NOGO | Heavy chain | 2A10 construct | WO2007003421 | 3914 |
| | | humanized construct | | SEQ ID NO: 28 | |
| | | H700 | | | |
| PDLB129 | RTN4 (NOGO) | Heavy chain IgG4, | Atinumab | U.S. Pat. No. 8,163,285 | 3915 |
| | | immunomodulator | | SEQ ID NO: 24 | |
| PDLB130 | alpha synuclein | Heavy chain | NI-202.12F4-VHA1b-GL | US20150232542 | 3916 |
| | | variable region | | SEQ ID NO: 10 | |
| PDLB131 | alpha synuclein | Heavy chain | NI-202.3D8-VHE1 | US20150232542 | 3917 |
| | | variable region | | SEQ ID NO: 15 | |
| PDLB132 | alpha synuclein | Heavy chain | NI-202.3D8-VHE1-GL | US20150232542 | 3918 |
| | | variable region | | SEQ ID NO: 16 | |
| PDLB133 | alpha synuclein | Heavy chain | NI-202.3G12-VHB1 | US20150232542 | 3919 |
| | | variable region | | SEQ ID NO: 3 | |
| PDLB134 | alpha synuclein | Heavy chain | NI-202.3G12-VHB1-GL | US20150232542 | 3920 |
| | | variable region | | SEQ ID NO: 4 | |
| PDLB135 | alpha synuclein | Heavy chain | NI-202.12F4-VHA1b | US20150232542 | 3921 |
| | | variable region | | SEQ ID NO: 9 | |
| PDLB136 | alpha synuclein | Heavy chain | Hu9E4VHv3 variable | U.S. Pat. No. 8,609,820 | 3922 |
| | | variable region | region | SEQ ID NO: 10 | |
| PDLB137 | alpha synuclein | Heavy chain | Hu9E4VHv4 variable | U.S. Pat. No. 8,609,820 | 3923 |
| | | variable region | region | SEQ ID NO: 11 | |
| PDLB138 | alpha synuclein | Heavy chain | Hu9E4VLv3 variable | U.S. Pat. No. 8,609,820 | 3924 |
| | | variable region | region | SEQ ID NO: 5 | |
| PDLB139 | alpha synuclein | Heavy chain | m9E4VH variable region | U.S. Pat. No. 8,609,820 | 3925 |
| | | variable region | | SEQ ID NO: 6 | |
| PDLB140 | alpha synuclein | Heavy chain | 1791009Hu9E4VHFr | U.S. Pat. No. 8,609,820 | 3926 |
| | | variable region | variable region | SEQ ID NO: 7 | |
| PDLB141 | alpha synuclein | Heavy chain | Hu9E4VHv1 variable | U.S. Pat. No. 8,609,820 | 3927 |
| | | variable region | region | SEQ ID NO: 8 | |
| PDLB142 | alpha synuclein | Heavy chain | Hu9E4VHv2 variable | U.S. Pat. No. 8,609,820 | 3928 |
| | | variable region | region | SEQ ID NO: 9 | |
| PDLB143 | alpha synuclein | Heavy chain | m1H7 | U.S. Pat. No. 8,790,644 | 3929 |
| | | variable region | | SEQ ID NO: 5 | |
| PDLB144 | alpha synuclein | Heavy chain | mature m1H7 | U.S. Pat. No. 8,790,644 | 3930 |
| | | variable region | | SEQ ID NO: 9 | |
| PDLB145 | alpha synuclein | Heavy chain | Hu9E4VHv 3 | WO2015075635 | 3931 |
| | | variable region | | SEQ ID NO: 10 | |
| PDLB146 | alpha synuclein | Heavy chain | Hu1H7VHv4 | WO2015075635 | 3932 |
| | | variable region | | SEQ ID NO: 101 | |
| PDLB147 | alpha synuclein | Heavy chain | Hu9E4VHv4 (no back | WO2015075635 | 3933 |
| | | variable region | mutation) | SEQ ID NO: 11 | |
| PDLB148 | alpha synuclein | Heavy chain | Hu1H7VHv5 | WO2015075635 | 3934 |
| | | variable region | | SEQ ID NO: 103 | |
| PDLB149 | alpha synuclein | Heavy chain | 63 102889Hu9E4VLFr | WO2015075635 | 3935 |
| | | variable region | | SEQ ID NO: 2 | |
| PDLB150 | alpha synuclein | Heavy chain | m5C1 antibody heavy | WO2015075635 | 3936 |
| | | variable region | chain variable region | SEQ ID NO: 39 | |
| | | | amino acid sequence | | |
| PDLB151 | alpha synuclein | Heavy chain | i 791009Hu9E4VHFr | WO2015075635 | 3937 |
| | | variable region | | SEQ ID NO: 7 | |
| PDLB152 | alpha synuclein | Heavy chain | Hu9E4VHv 1 | WO2015075635 | 3938 |
| | | variable region | | SEQ ID NO: 8 | |
| PDLB153 | alpha synuclein | Heavy chain | mlH7 | WO2015075635 | 3939 |
| | | variable region | | SEQ ID NO: 81 | |
| PDLB154 | alpha synuclein | Heavy chain | mature mlH7 | WO2015075635 | 3940 |
| | | variable region | | SEQ ID NO: 85 | |
| PDLB155 | alpha synuclein | Heavy chain | Hu9E4VHv 2 | WO2015075635 | 3941 |
| | | variable region | | SEQ ID NO: 9 | |
| PDLB156 | alpha synuclein | Heavy chain | Hu lH7VHv1 | WO2015075635 | 3942 |
| | | variable region | | SEQ ID NO: 95 | |
| PDLB157 | alpha synuclein | Heavy chain | Hu1H7VHv2 | WO2015075635 | 3943 |
| | | variable region | | SEQ ID NO: 97 | |
| PDLB158 | alpha synuclein | Heavy chain | Hu1H7VHv3 | WO2015075635 | 3944 |
| | | variable region | | SEQ ID NO: 99 | |
| PDLB159 | alpha synuclein | Heavy chain | BA1: 49/G | WO2011104696 | 3945 |
| | protofibrils | variable region | | SEQ ID NO: 56 | |
| PDLB160 | alpha synuclein | Heavy chain | BA1: 49/G | WO2011104696 | 3946 |
| | protofibrils | variable region | | SEQ ID NO: 57 | |
| PDLB161 | alpha synuclein | Heavy chain | BA2: 38E2/7 | WO2011104696 | 3947 |
| | protofibrils | variable region | | SEQ ID NO: 58 | |
| PDLB162 | alpha synuclein | Heavy chain | BA2: 38E2/7 | WO2011104696 | 3948 |
| | protofibrils | variable region | | SEQ ID NO: 59 | |
| PDLB163 | amyloid | Heavy chain | F11G3 | U.S. Pat. No. 9,125,846 | 3949 |
| | oligomers | variable region | | SEQ ID NO: 11 | |
| PDLB164 | DR6 and P75 | Heavy chain | M66-B03 | WO2010062904 | 3950 |
| | | variable region | | SEQ ID NO: 67 | |
| PDLB165 | DR6 and P75 | Heavy chain | M50-H01 | WO2010062904 | 3951 |
| | | variable region | | SEQ ID NO: 7 | |
| PDLB166 | DR6 and P75 | Heavy chain | M67-G02 | WO2010062904 | 3952 |
| | | variable region | | SEQ ID NO: 77 | |
| PDLB167 | DR6 and P75 | Heavy chain | M72-F03 | WO2010062904 | 3953 |
| | | variable region | | SEQ ID NO: 87 | |
| PDLB168 | DR6 and P75 | Heavy chain | M73-C04 | WO2010062904 | 3954 |
| | | variable region | | SEQ ID NO: 97 | |
| PDLB169 | DR6 and P75 | Heavy chain | 1P1D6.3 | WO2010062904 | 3955 |
| | | variable region | | SEQ ID NO: 107 | |
| PDLB170 | DR6 and P75 | Heavy chain | 1P2F2.1 | WO2010062904 | 3956 |
| | | variable region | | SEQ ID NO: 117 | |
| PDLB171 | DR6 and P75 | Heavy chain | 1P5D10.2 | WO2010062904 | 3957 |
| | | variable region | | SEQ ID NO: 127 | |
| PDLB172 | DR6 and P75 | Heavy chain | M51-H09 | WO2010062904 | 3958 |
| | | variable region | | SEQ ID NO: 17 | |
| PDLB173 | DR6 and P75 | Heavy chain | M53-E04 | WO2010062904 | 3959 |
| | | variable region | | SEQ ID NO: 27 | |
| PDLB174 | DR6 and P75 | Heavy chain | M53-F04 | WO2010062904 | 3960 |
| | | variable region | | SEQ ID NO: 37 | |
| PDLB175 | DR6 and P75 | Heavy chain | M62-B02 | WO2010062904 | 3961 |
| | | variable region | | SEQ ID NO: 47 | |
| PDLB176 | DR6 and P75 | Heavy chain | M63-E10 | WO2010062904 | 3962 |
| | | variable region | | SEQ ID NO: 57 | |
| PDLB177 | LPG | Heavy chain | #7 | U.S. Pat. No. 8,591,902 | 3963 |
| | (lysophosphatidyl | variable region | | SEQ ID NO: 18 | |
| | glucoside) | | | | |
| PDLB178 | LPG | Heavy chain | #15 | U.S. Pat. No. 8,591,902 | 3964 |
| | (lysophosphatidyl | variable region | | SEQ ID NO: 8 | |
| | glucoside) | | | | |
| PDLB179 | MAG | Heavy chain | | U.S. Pat. No. 8,071,731 | 3965 |
| | | variable region | | SEQ ID NO: 13 | |
| PDLB180 | MAG | Heavy chain | | U.S. Pat. No. 8,071,731 | 3966 |
| | | variable region | | SEQ ID NO: 14 | |
| PDLB181 | MAG | Heavy chain | | U.S. Pat. No. 8,071,731 | 3967 |
| | | variable region | | SEQ ID NO: 15 | |
| PDLB182 | MAI (myelin | Heavy chain | | WO2013158748 | 3968 |
| | associated | variable region | | SEQ ID NO: 1 | |
| | inhibitor) | | | | |
| PDLB183 | MAI (myelin | Heavy chain | | WO2013158748 | 3969 |
| | associated | variable region | | SEQ ID NO: 17 | |
| | inhibitor) | | | | |
| PDLB184 | NMDA | Heavy chain | | EP2805972 | 3970 |
| | | variable region | | SEQ ID NO: 43 | |
| PDLB185 | NOGO | Heavy chain | H5L13, H5L16, H5L18, | US20140147435 | 3971 |
| | | variable region | H5L14, H5L15, H5L17, | SEQ ID NO: 11 | |
| | | | H5L6, H5L11 | | |
| PDLB186 | NOGO | Heavy chain | H6L13, H6L16, H6L18, | US20140147435 | 3972 |
| | | variable region | H6L14, H6L15, H6L17, | SEQ ID NO: 12 | |
| | | | H6L6 | | |
| PDLB187 | NOGO | Heavy chain | H700L13, H700L16, | US20140147435 | 3973 |
| | | variable region | H700L18, H700L14, | SEQ ID NO: 13 | |
| | | | H700L15, H700L17, | | |
| | | | H700L6, H700L11 | | |
| PDLB188 | NOGO | Heavy chain | H14L13, H14L16, | US20140147435 | 3974 |
| | | variable region | H14L18, H14L14, | SEQ ID NO: 14 | |
| | | | H14L15, H14L17, H14L6, | | |
| | | | H14L11 | | |
| PDLB189 | NOGO | Heavy chain | H15L13, H15L16, | US20140147435 | 3975 |
| | | variable region | H15L18, H15L14, | SEQ ID NO: 15 | |
| | | | H15L15, H15L17, H15L6, | | |
| | | | H15L11 | | |
| PDLB190 | NOGO | Heavy chain | H16L13, H16L16, | US20140147435 | 3976 |
| | | variable region | H16L18, H16L14, | SEQ ID NO: 16 | |
| | | | H16L15, H16L17, H16L6, | | |
| | | | H16L11 | | |
| PDLB191 | NOGO | Heavy chain | H17L13, H17L16, | US20140147435 | 3977 |
| | | variable region | H17L18, H17L14, | SEQ ID NO: 17 | |
| | | | H17L15, H17L17, H17L6, | | |
| | | | H17L11 | | |
| PDLB192 | NOGO | Heavy chain | H18L13, H18L16, | US20140147435 | 3978 |
| | | variable region | H18L18, H18L14, | SEQ ID NO: 18 | |
| | | | H18L15, H18L17, H18L6, | | |
| | | | H18L11 | | |
| PDLB193 | NOGO | Heavy chain | H1L13, H1L16, H1L18, | US20140147435 | 3979 |
| | | variable region | H1L14, H1L15, H1L17, | SEQ ID NO: 77 | |
| | | | H1L6 | | |
| PDLB194 | NOGO | Heavy chain | H19L13, H19L16, | US20140147435 | 3980 |
| | | variable region | H19L18, H19L4, | SEQ ID NO: 85 | |
| | | | H19L15, H19L17, H19L6, | | |
| | | | H19L11 | | |
| PDLB195 | NOGO | Heavy chain | H20L13, H20L16, | US20140147435 | 3981 |
| | | variable region | H20L18, H20L14, | SEQ ID NO: 86 | |
| | | | H20L15, H20L17, H20L6, | | |
| | | | H20L11 | | |
| PDLB196 | NOGO | Heavy chain | H21L13, H21L16, | US20140147435 | 3982 |
| | | variable region | H21L18, H21L14, | SEQ ID NO: 87 | |
| | | | H21L15, H21L17, H21L6, | | |
| | | | H21L11 | | |
| PDLB197 | NOGO | Heavy chain | H22L13, H22L16, | US20140147435 | 3983 |
| | | variable region | H22L18, H22L14, | SEQ ID NO: 88 | |
| | | | H22L15, H22L17, H22L6, | | |
| | | | H22L11 | | |
| PDLB198 | NOGO | Heavy chain | H23L13, H23L16, | US20140147435 | 3984 |
| | | variable region | H23L18, H23L14, | SEQ ID NO: 89 | |
| | | | H23L15, H23L17, H23L6, | | |
| | | | H23L11 | | |
| PDLB199 | NOGO | Heavy chain | H24L13, H24L16, | US20140147435 | 3985 |
| | | variable region | H24L18, H24L14, | SEQ ID NO: 90 | |
| | | | H24L15, H24L17, H24L6, | | |
| | | | H24L11 | | |
| PDLB200 | NOGO | Heavy chain | H25L13, H25L16, | US20140147435 | 3986 |
| | | variable region | H25L18, H25L14, | SEQ ID NO: 91 | |
| | | | H25L15, H25L17, H25L6, | | |
| | | | H25L11 | | |
| PDLB201 | Nogo-66 | Heavy chain | Antibody clone 50 | US20140065155 | 3987 |
| | | variable region | | SEQ ID NO: 3 | |
| PDLB202 | Nogo-66 | Heavy chain | Antibody clone 51 | US20140065155 | 3988 |
| | | variable region | | SEQ ID NO: 5 | |
| PDLB203 | NogoA/NiG | Heavy chain | 6A3-Ig4 | WO2009056509 | 3989 |
| | | variable region | | SEQ ID NO: 24 | |
| PDLB204 | NogoA/NiG | Heavy chain | 6A3-IgG1 | WO2009056509 | 3990 |
| | | variable region | | SEQ ID NO: 4 | |
| PDLB205 | RGM A | Heavy chain | 5F9.1-GL | US20150183871 | 3991 |
| | | variable region | | SEQ ID NO: 35 | |
| PDLB206 | RGM A | Heavy chain | 5F9.2-GL | US20150183871 | 3992 |
| | | variable region | | SEQ ID NO: 36 | |
| PDLB207 | RGM A | Heavy chain | 5F9.3-GL | US20150183871 | 3993 |
| | | variable region | | SEQ ID NO: 37 | |
| PDLB208 | RGM A | Heavy chain | 5F9.4-GL | US20150183871 | 3994 |
| | | variable region | | SEQ ID NO: 38 | |
| PDLB209 | RGM A | Heavy chain | 5F9.5-GL | US20150183871 | 3995 |
| | | variable region | | SEQ ID NO: 39 | |
| PDLB210 | RGM A | Heavy chain | 5F9.6-GL | US20150183871 | 3996 |
| | | variable region | | SEQ ID NO: 40 | |
| PDLB211 | RGM A | Heavy chain | 5F9.7-GL | US20150183871 | 3997 |
| | | variable region | | SEQ ID NO: 41 | |
| PDLB212 | RGM A | Heavy chain | 5F9.8-GL | US20150183871 | 3998 |
| | | variable region | | SEQ ID NO: 42 | |
| PDLB213 | RGM A | Heavy chain | 5F9.9-GL | US20150183871 | 3999 |
| | | variable region | | SEQ ID NO: 43 | |
| PDLB214 | RGM A | Heavy chain | h5F9.1, h5F9.1, h5F9.1, | US20150183871 | 4000 |
| | | variable region | h5F9.1, h5F9.1, h5F9.2, | SEQ ID NO: 47 | |
| | | | h5F9.3 | | |
| PDLB215 | RGM A | Heavy chain | h5F9.3, h5F9.9, h5F9.25 | US20150183871 | 4001 |
| | | variable region | | SEQ ID NO: 53 | |
| PDLB216 | RGM A | Heavy chain | h5F9.4, h5F9.10, h5F9.26 | US20150183871 | 4002 |
| | | variable region | | SEQ ID NO: 54 | |
| PDLB217 | RGMa | Heavy chain | AE12-1 | US20140023659 | 4003 |
| | | variable region | | SEQ ID NO: 1 | |
| PDLB218 | RGMa | Heavy chain | AE12-20 | US20140023659 | 4004 |
| | | variable region | | SEQ ID NO: 107 | |
| PDLB219 | RGMa | Heavy chain | AE12-21 | US20140023659 | 4005 |
| | | variable region | | SEQ ID NO: 115 | |
| PDLB220 | RGMa | Heavy chain | AE12-23 | US20140023659 | 4006 |
| | | variable region | | SEQ ID NO: 123 | |
| PDLB221 | RGMa | Heavy chain | AE12-24 | US20140023659 | 4007 |
| | | variable region | | SEQ ID NO: 131 | |
| PDLB222 | RGMa | Heavy chain | AE12-3 | US20140023659 | 4008 |
| | | variable region | | SEQ ID NO: 17 | |
| PDLB223 | RGMa | Heavy chain | AE12-4 | US20140023659 | 4009 |
| | | variable region | | SEQ ID NO: 25 | |
| PDLB224 | RGMa | Heavy chain | AE12-5 | US20140023659 | 4010 |
| | | variable region | | SEQ ID NO: 33 | |
| PDLB225 | RGMa | Heavy chain | AE12-6 | US20140023659 | 4011 |
| | | variable region | | SEQ ID NO: 41 | |
| PDLB226 | RGMa | Heavy chain | AE12-7 | US20140023659 | 4012 |
| | | variable region | | SEQ ID NO: 49 | |
| PDLB227 | RGMa | Heavy chain | AE12-8 | US20140023659 | 4013 |
| | | variable region | | SEQ ID NO: 57 | |
| PDLB228 | RGMa | Heavy chain | AE12-2 | US20140023659 | 4014 |
| | | variable region | | SEQ ID NO: 9 | |
| PDLB229 | RGMa | Heavy chain | AE12-13 | US20140023659 | 4015 |
| | | variable region | | SEQ ID NO: 91 | |
| PDLB230 | RGMa | Heavy chain | AE12-15 | US20140023659 | 4016 |
| | | variable region | | SEQ ID NO: 99 | |
| PDLB231 | α-synuclein | Heavy chain | Syn-01 | WO2014132210 | 4017 |
| | aggregates | variable region | | SEQ ID NO: 10 | |
| PDLB232 | α-synuclein | Heavy chain | Syn-F1 | WO2014132210 | 4018 |
| | aggregates | variable region | | SEQ ID NO: 2 | |
| PDLB233 | α-synuclein | Heavy chain | Syn-F2 | WO2014132210 | 4019 |
| | aggregates | variable region | | SEQ ID NO: 6 | |
| PDLB234 | NOGO | Heavy chain | 2A10 construct | WO2007003421 | 4020 |
| | | variable region | | SEQ ID NO: 77 | |
| | | humanized construct | | | |
| | | H1 | | | |
| PDLB235 | NOGO | Heavy chain | 2A10 construct | WO2007003421 | 4021 |
| | | variable region | | SEQ ID NO: 14 | |
| | | humanized construct | | | |
| | | H14 | | | |
| PDLB236 | NOGO | Heavy chain | 2A10 construct | WO2007003421 | 4022 |
| | | variable region | | SEQ ID NO: 15 | |
| | | humanized construct | | | |
| | | H15 | | | |
| PDLB237 | NOGO | Heavy chain | 2A10 construct | WO2007003421 | 4023 |
| | | variable region | | SEQ ID NO: 16 | |
| | | humanized construct | | | |
| | | H16 | | | |
| PDLB238 | NOGO | Heavy chain | 2A10 construct | WO2007003421 | 4024 |
| | | variable region | | SEQ ID NO: 17 | |
| | | humanized construct | | | |
| | | H17 | | | |
| PDLB239 | NOGO | Heavy chain | 2A10 construct | WO2007003421 | 4025 |
| | | variable region | | SEQ ID NO: 18 | |
| | | humanized construct | | | |
| | | H18 | | | |
| PDLB240 | NOGO | Heavy chain | 2A10 construct | WO2007003421 | 4026 |
| | | variable region | | SEQ ID NO: 85 | |
| | | humanized construct | | | |
| | | H19 | | | |
| PDLB241 | NOGO | Heavy chain | 2A10 construct | WO2007003421 | 4027 |
| | | variable region | | SEQ ID NO: 86 | |
| | | humanized construct | | | |
| | | H20 | | | |
| PDLB242 | NOGO | Heavy chain | 2A10 construct | WO2007003421 | 4028 |
| | | variable region | | SEQ ID NO: 87 | |
| | | humanized construct | | | |
| | | H21 | | | |
| PDLB243 | NOGO | Heavy chain | 2A10 construct | WO2007003421 | 4029 |
| | | variable region | | SEQ ID NO: 88 | |
| | | humanized construct | | | |
| | | H22 | | | |
| PDLB244 | NOGO | Heavy chain | 2A10 construct | WO2007003421 | 4030 |
| | | variable region | | SEQ ID NO: 89 | |
| | | humanized construct | | | |
| | | H23 | | | |
| PDLB245 | NOGO | Heavy chain | 2A10 construct | WO2007003421 | 4031 |
| | | variable region | | SEQ ID NO: 90 | |
| | | humanized construct | | | |
| | | H24 | | | |
| PDLB246 | NOGO | Heavy chain | 2A10 construct | WO2007003421 | 4032 |
| | | variable region | | SEQ ID NO: 91 | |
| | | humanized construct | | | |
| | | H25 | | | |
| PDLB247 | NOGO | Heavy chain | 2A10 construct | WO2007003421 | 4033 |
| | | variable region | | SEQ ID NO: 11 | |
| | | humanized construct | | | |
| | | H5 | | | |
| PDLB248 | NOGO | Heavy chain | 2A10 construct | WO2007003421 | 4034 |
| | | variable region | | SEQ ID NO: 12 | |
| | | humanized construct | | | |
| | | H6 | | | |
| PDLB249 | NOGO | Heavy chain | 2A10 construct | WO2007003421 | 4035 |
| | | variable region | | SEQ ID NO: 13 | |
| | | humanized construct | | | |
| | | H700 | | | |
| PDLB250 | alpha synuclein | Heavy chain version | Hu1H7 | U.S. Pat. No. 8,790,644 | 4036 |
| | | 3 (variable | | SEQ ID NO: 55 | |
| | | region + constant | | | |
| | | region) | | | |
| PDLB251 | alpha synuclein | Heavy chain version | Hu1H7 | U.S. Pat. No. 8,790,644 | 4037 |
| | | 3 (variable | | SEQ ID NO: 56 | |
| | | region + constant | | | |
| | | region; G1m3 | | | |
| | | allotype) | | | |
| PDLB252 | 118-126 of α- | Light chain | 5ClL1 | US20150259404 | 4038 |
| | synuclein | | | SEQ ID NO: 29 | |
| PDLB253 | 118-126 of α- | Light chain | 5ClL2 | US20150259404 | 4039 |
| | synuclein | | | SEQ ID NO: 30 | |
| PDLB254 | 118-126 of α- | Light chain | 5ClL3 | US20150259404 | 4040 |
| | synuclein | | | SEQ ID NO: 31 | |
| PDLB255 | 118-126 of α- | Light chain | 5ClL4 | US20150259404 | 4041 |
| | synuclein | | | SEQ ID NO: 32 | |
| PDLB256 | 118-126 of α- | Light chain | IgG1 | US20150259404 | 4042 |
| | synuclein | | | SEQ ID NO: 40 | |
| PDLB257 | 118-126 of α- | Light chain | 5Cl | US20150259404 | 4043 |
| | synuclein | | | SEQ ID NO: 8 | |
| PDLB258 | ACTH | Light chain | Ab3 | WO2015127288 | 4044 |
| | | | | SEQ ID NO: 101 | |
| PDLB259 | ACTH | Light chain | Ab4 | WO2015127288 | 4045 |
| | | | | SEQ ID NO: 141 | |
| PDLB260 | ACTH | Light chain | Ab5 | WO2015127288 | 4046 |
| | | | | SEQ ID NO: 181 | |
| PDLB261 | ACTH | Light chain | Ab1 | WO2015127288 | 4047 |
| | | | | SEQ ID NO: 21 | |
| PDLB262 | ACTH | Light chain | Ab6 | WO2015127288 | 4048 |
| | | | | SEQ ID NO: 221 | |
| PDLB263 | ACTH | Light chain | Ab7 | WO2015127288 | 4049 |
| | | | | SEQ ID NO: 261 | |
| PDLB264 | ACTH | Light chain | Ab9 | WO2015127288 | 4050 |
| | | | | SEQ ID NO: 301 | |
| PDLB265 | ACTH | Light chain | Ab10 | WO2015127288 | 4051 |
| | | | | SEQ ID NO: 341 | |
| PDLB266 | ACTH | Light chain | Ab11 | WO2015127288 | 4052 |
| | | | | SEQ ID NO: 381 | |
| PDLB267 | ACTH | Light chain | Ab12 | WO2015127288 | 4053 |
| | | | | SEQ ID NO: 421 | |
| PDLB268 | ACTH | Light chain | Ab1.H | WO2015127288 | 4054 |
| | | | | SEQ ID NO: 461 | |
| PDLB269 | ACTH | Light chain | Ab2.H | WO2015127288 | 4055 |
| | | | | SEQ ID NO: 501 | |
| PDLB270 | ACTH | Light chain | Ab3.H | WO2015127288 | 4056 |
| | | | | SEQ ID NO: 541 | |
| PDLB271 | ACTH | Light chain | Ab4.H | WO2015127288 | 4057 |
| | | | | SEQ ID NO: 581 | |
| PDLB272 | ACTH | Light chain | Ab2 | WO2015127288 | 4058 |
| | | | | SEQ ID NO: 61 | |
| PDLB273 | ACTH | Light chain | Ab6.H | WO2015127288 | 4059 |
| | | | | SEQ ID NO: 621 | |
| PDLB274 | ACTH | Light chain | Ab7.H | WO2015127288 | 4060 |
| | | | | SEQ ID NO: 661 | |
| PDLB275 | ACTH | Light chain | Ab7A.H | WO2015127288 | 4061 |
| | | | | SEQ ID NO: 701 | |
| PDLB276 | ACTH | Light chain | Ab10.H | WO2015127288 | 4062 |
| | | | | SEQ ID NO: 741 | |
| PDLB277 | ACTH | Light chain | Ab11.H | WO2015127288 | 4063 |
| | | | | SEQ ID NO: 781 | |
| PDLB278 | ACTH | Light chain | Ab11A.H | WO2015127288 | 4064 |
| | | | | SEQ ID NO: 821 | |
| PDLB279 | ACTH | Light chain | Ab12.H | WO2015127288 | 4065 |
| | | | | SEQ ID NO: 861 | |
| PDLB280 | alpha synuclein | Light chain | Hu1H7VLv1 | U.S. Pat. No. 8,790,644 | 4066 |
| | | | | SEQ ID NO: 33 | |
| PDLB281 | alpha synuclein | Light chain | Hu1H7VLv2 | U.S. Pat. No. 8,790,644 | 4067 |
| | | | | SEQ ID NO: 35 | |
| PDLB282 | alpha synuclein | Light chain | Hu1H7VLv3 | U.S. Pat. No. 8,790,644 | 4068 |
| | | | | SEQ ID NO: 37 | |
| PDLB283 | alpha synuclein | Light chain | Hu1H7VLv4 | U.S. Pat. No. 8,790,644 | 4069 |
| | | | | SEQ ID NO: 39 | |
| PDLB284 | alpha synuclein | Light chain | Hu1H7VL alternative | U.S. Pat. No. 8,790,644 | 4070 |
| | | | | SEQ ID NO: 45 | |
| PDLB285 | alpha synuclein | Light chain | sequence for Hu1H7VL | U.S. Pat. No. 8,790,644 | 4071 |
| | | | alternatives | SEQ ID NO: 47 | |
| PDLB286 | alpha synuclein | Light chain | humanized 5C 1L1 | WO2015075635 | 4072 |
| | | | | SEQ ID NO: 69 | |
| PDLB287 | alpha synuclein | Light chain | humanized 5C 1L2 | WO2015075635 | 4073 |
| | | | | SEQ ID NO: 70 | |
| PDLB288 | alpha synuclein | Light chain | Hu1H7VL alternative | WO2015075635 | 4074 |
| | | | | SEQ ID NO: 122 | |
| PDLB289 | alpha synuclein | Light chain | humanized 1H7 light chain | WO2015075635 | 4075 |
| | | | version 3 (variable region + | SEQ ID NO: 124 | |
| | | | constant region with | | |
| | | | Arginine) | | |
| PDLB290 | alpha synuclein | Light chain | humanized 1H7 light chain | WO2015075635 | 4076 |
| | | | version 3 (variable region + | SEQ ID NO: 125 | |
| | | | constant region without | | |
| | | | Arginine) | | |
| PDLB291 | alpha synuclein | Light chain | Hu9E4VL alternative | WO2015075635 | 4077 |
| | | | | SEQ ID NO: 28 | |
| PDLB292 | alpha synuclein | Light chain | humanized 9E4 light chain | WO2015075635 | 4078 |
| | | | version 3 (variable region + | SEQ ID NO: 32 | |
| | | | constant region with | | |
| | | | Arginine) | | |
| PDLB293 | alpha synuclein | Light chain | humanized 9E4 light chain | WO2015075635 | 4079 |
| | | | version 3 (variable region + | SEQ ID NO: 33 | |
| | | | constant region without | | |
| | | | Arginine) | | |
| PDLB294 | alpha synuclein | Light chain | humanized 5C L3 | WO2015075635 | 4080 |
| | | | | SEQ ID NO: 71 | |
| PDLB295 | amyloids | Light chain | #118 | WO2010012004 | 4081 |
| | | | | SEQ ID NO: 10 | |
| PDLB296 | amyloids | Light chain | #121 | WO2010012004 | 4082 |
| | | | | SEQ ID NO: 12 | |
| PDLB297 | amyloids | Light chain | #201 | WO2010012004 | 4083 |
| | | | | SEQ ID NO: 14 | |
| PDLB298 | amyloids | Light chain | #204 | WO2010012004 | 4084 |
| | | | | SEQ ID NO: 15 | |
| PDLB299 | amyloids | Light chain | #205 | WO2010012004 | 4085 |
| | | | | SEQ ID NO: 17 | |
| PDLB300 | EAG1 | Light chain | chimeric ImAb3 | WO2006037604 | 4086 |
| | | | | SEQ ID NO: 10 | |
| PDLB301 | EAG1 | Light chain | chimeric ImAb4 | WO2006037604 | 4087 |
| | | | | SEQ ID NO: 14 | |
| PDLB302 | EAG1 | Light chain | LC-lmAb3-humB3 | WO2006037604 | 4088 |
| | | | | SEQ ID NO: 18 | |
| PDLB303 | EAG1 | Light chain | ImAb4 | WO2006037604 | 4089 |
| | | | | SEQ ID NO: 2 | |
| PDLB304 | EAG1 | Light chain | LC-lmAb4-humA17 | WO2006037604 | 4090 |
| | | | | SEQ ID NO: 22 | |
| PDLB305 | EAG1 | Light chain | LC-lmAb3-humA3 | WO2006037604 | 4091 |
| | | | | SEQ ID NO: 26 | |
| PDLB306 | EAG1 | Light chain | LC-lmAb3-humA17 | WO2006037604 | 4092 |
| | | | | SEQ ID NO: 30 | |
| PDLB307 | EAG1 | Light chain | LC-lmAb4-humA5-1 | WO2006037604 | 4093 |
| | | | | SEQ ID NO: 34 | |
| PDLB308 | EAG1 | Light chain | LC-lmAh4-humO1 | WO2006037604 | 4094 |
| | | | | SEQ ID NO: 38 | |
| PDLB309 | EAG1 | Light chain | ImAb3 | WO2006037604 | 4095 |
| | | | | SEQ ID NO: 6 | |
| PDLB310 | NOGO | Light chain | H6L13 FL, H19L13 FL, | US20140147435 | 4096 |
| | | | H20L13 FL, H21L13 FL, | SEQ ID NO: 35 | |
| | | | H25L13 FL | | |
| PDLB311 | NOGO | Light chain | H16L16 FL, H19L16 FL, | US20140147435 | 4097 |
| | | | H20L16 FL, H21L16 FL, | SEQ ID NO: 38 | |
| | | | H25L16 FL, H18L16 FL | | |
| PDLB312 | NOGO | Light chain | H16L18 FL, H19L18 FL, | US20140147435 | 4098 |
| | | | H20L18 FL, H21L18 FL, | SEQ ID NO: 40 | |
| | | | H25L18 FL | | |
| PDLB313 | Nogo receptor-1 | Light chain | 7E11 | US20090215691 | 4099 |
| | | | | SEQ ID NO: 15 | |
| PDLB314 | Nogo receptor-1 | Light chain | 7E11 | US20090215691 | 4100 |
| | | | | SEQ ID NO: 17 | |
| PDLB315 | trk-C (NT-3 trkC | Light chain | 2250 | U.S. Pat. No. 7,615,383 | 4101 |
| | ligand) | | | SEQ ID NO: 49 | |
| PDLB316 | trk-C (NT-3 trkC | Light chain | 2253 | U.S. Pat. No. 7,615,383 | 4102 |
| | ligand) | | | SEQ ID NO: 50 | |
| PDLB317 | trk-C (NT-3 trkC | Light chain | 2256 | U.S. Pat. No. 7,615,383 | 4103 |
| | ligand) | | | SEQ ID NO: 51 | |
| PDLB318 | trk-C (NT-3 trkC | Light chain | 6.1.2 | U.S. Pat. No. 7,615,383 | 4104 |
| | ligand) | | | SEQ ID NO: 52 | |
| PDLB319 | trk-C (NT-3 trkC | Light chain | 6.4.1 | U.S. Pat. No. 7,615,383 | 4105 |
| | ligand) | | | SEQ ID NO: 53 | |
| PDLB320 | trk-C (NT-3 trkC | Light chain | 2345 | U.S. Pat. No. 7,615,383 | 4106 |
| | ligand) | | | SEQ ID NO: 54 | |
| PDLB321 | trk-C (NT-3 trkC | Light chain | 2349 | U.S. Pat. No. 7,615,383 | 4107 |
| | ligand) | | | SEQ ID NO: 55 | |
| PDLB322 | alpha synuclein | Light chain | Hu9E4VL consensus | U.S. Pat. No. 8,609,820 | 4108 |
| | | consensus chain | amino acid sequence | SEQ ID NO: 26 | |
| PDLB323 | alpha synuclein | Light chain constant | humanized 9E4 light chain | U.S. Pat. No. 8,609,820 | 4109 |
| | | region | constant region | SEQ ID NO: 13 | |
| PDLB324 | alpha synuclein | Light chain constant | humanized 9E4 heavy | U.S. Pat. No. 8,609,820 | 4110 |
| | | region | chain constant region | SEQ ID NO: 14 | |
| PDLB325 | alpha synuclein | Light chain constant | humanized 9E4 | WO2015075635 | 4111 |
| | | region | | SEQ ID NO: 13 | |
| PDLB326 | alpha synuclein | Light chain constant | Hu1H7 | U.S. Pat. No. 8,790,644 | 4112 |
| | | region (with | | SEQ ID NO: 49 | |
| | | arginine) (common | | | |
| | | for v1-v4) | | | |
| PDLB327 | alpha synuclein | Light chain constant | Hu1H7 | U.S. Pat. No. 8,790,644 | 4113 |
| | | region (without | | SEQ ID NO: 51 | |
| | | arginine) (common | | | |
| | | for v1-v4) | | | |
| PDLB328 | many - growth | Light chain fusion | H21L13, H21L16, | U.S. Pat. No. 8,053,569 | 4114 |
| | factors (to | protein | H21L18, H21L14, | SEQ ID NO: 31 | |
| | increase transport | | H21L15, H21L17, H21L6, | | |
| | across BBB) | | H21L11 | | |
| PDLB329 | many - growth | Light chain fusion | H23L13, H23L16, | U.S. Pat. No. 8,053,569 | 4115 |
| | factors (to | protein | H23L18, H23L14, | SEQ ID NO: 36 | |
| | increase transport | | H23L15, H23L17, H23L6, | | |
| | across BBB) | | H23L11 | | |
| PDLB330 | NOGO | Light chain | 2A10 construct | WO2007003421 | 4116 |
| | | humanized construct | | SEQ ID NO: 80 | |
| | | L11 | | | |
| PDLB331 | NOGO | Light chain | 2A10 construct | WO2007003421 | 4117 |
| | | humanized construct | | SEQ ID NO: 35 | |
| | | L13 | | | |
| PDLB332 | NOGO | Light chain | 2A10 construct | WO2007003421 | 4118 |
| | | humanized construct | | SEQ ID NO: 36 | |
| | | L14 | | | |
| PDLB333 | NOGO | Light chain | 2A10 construct | WO2007003421 | 4119 |
| | | humanized construct | | SEQ ID NO: 37 | |
| | | L15 | | | |
| PDLB334 | NOGO | Light chain | 2A10 construct | WO2007003421 | 4120 |
| | | humanized construct | | SEQ ID NO: 38 | |
| | | L16 | | | |
| PDLB335 | NOGO | Light chain | 2A10 construct | WO2007003421 | 4121 |
| | | humanized construct | | SEQ ID NO: 39 | |
| | | L17 | | | |
| PDLB336 | NOGO | Light chain | 2A10 construct | WO2007003421 | 4122 |
| | | humanized construct | | SEQ ID NO: 40 | |
| | | L18 | | | |
| PDLB337 | NOGO | Light chain | 2A10 construct | WO2007003421 | 4123 |
| | | humanized construct | | SEQ ID NO: 34 | |
| | | L6 | | | |
| PDLB338 | RTN4 | Light chain IgG4, | Atinumab | U.S. Pat. No. 8,163,285 | 4124 |
| | | immunomodulator | | SEQ ID NO: 25 | |
| PDLB339 | alpha synuclein | Light chain variable | NI-202.12F4-VLa1 | US20150232542 | 4125 |
| | | region | | SEQ ID NO: 12 | |
| PDLB340 | alpha synuclein | Light chain variable | NI-202.12F4-VLa1-GL | US20150232542 | 4126 |
| | | region | | SEQ ID NO: 13 | |
| PDLB341 | alpha synuclein | Light chain variable | NI-202.3D8-VKa1 | US20150232542 | 4127 |
| | | region | | SEQ ID NO: 18 | |
| PDLB342 | alpha synuclein | Light chain variable | NI-202.3D8-VKa1-GL | US20150232542 | 4128 |
| | | region | | SEQ ID NO: 19 | |
| PDLB343 | alpha synuclein | Light chain variable | NI-202.3D8-VKc1 | US20150232542 | 4129 |
| | | region | | SEQ ID NO: 21 | |
| PDLB344 | alpha synuclein | Light chain variable | NI-202.3D8-VKc1-GL | US20150232542 | 4130 |
| | | region | | SEQ ID NO: 22 | |
| PDLB345 | alpha synuclein | Light chain variable | NI-202.3G12-VLc1 | US20150232542 | 4131 |
| | | region | | SEQ ID NO: 6 | |
| PDLB346 | alpha synuclein | Light chain variable | NI-202.3G12-VLc1-GL | US20150232542 | 4132 |
| | | region | | SEQ ID NO: 7 | |
| PDLB347 | alpha synuclein | Light chain variable | m9E4VL variable region | U.S. Pat. No. 8,609,820 | 4133 |
| | | region | | SEQ ID NO: 1 | |
| PDLB348 | alpha synuclein | Light chain variable | 63102889Hu9E4VLFr | U.S. Pat. No. 8,609,820 | 4134 |
| | | region | region variable | SEQ ID NO: 2 | |
| PDLB349 | alpha synuclein | Light chain variable | Hu9E4VLv1 variable | U.S. Pat. No. 8,609,820 | 4135 |
| | | region | region | SEQ ID NO: 3 | |
| PDLB350 | alpha synuclein | Light chain variable | Hu9E4VLv2 variable | U.S. Pat. No. 8,609,820 | 4136 |
| | | region | region | SEQ ID NO: 4 | |
| PDLB351 | alpha synuclein | Light chain variable | mature m1H7 light chain | U.S. Pat. No. 8,790,644 | 4137 |
| | | region | variable | SEQ ID NO: 11 | |
| PDLB352 | alpha synuclein | Light chain variable | m1H7 light chain variable | U.S. Pat. No. 8,790,644 | 4138 |
| | | region | | SEQ ID NO: 7 | |
| PDLB353 | alpha synuclein | Light chain variable | m9E4VL | WO2015075635 | 4139 |
| | | region | | SEQ ID NO: 1 | |
| PDLB354 | alpha synuclein | Light chain variable | Hu1H7VLv2 | WO2015075635 | 4140 |
| | | region | | SEQ ID NO: 111 | |
| PDLB355 | alpha synuclein | Light chain variable | Hu1H7VLv3 | WO2015075635 | 4141 |
| | | region | | SEQ ID NO: 113 | |
| PDLB356 | alpha synuclein | Light chain variable | Hu1H7VLv1 | WO2015075635 | 4142 |
| | | region | | SEQ ID NO: 109 | |
| PDLB357 | alpha synuclein | Light chain variable | Hu1H7VLv4 | WO2015075635 | 4143 |
| | | region | | SEQ ID NO: 115 | |
| PDLB358 | alpha synuclein | Light chain variable | Hu9E4VLv1 | WO2015075635 | 4144 |
| | | region | | SEQ ID NO: 3 | |
| PDLB359 | alpha synuclein | Light chain variable | Hu9E4VLv2 (No back | WO2015075635 | 4145 |
| | | region | mutation) | SEQ ID NO: 4 | |
| PDLB360 | alpha synuclein | Light chain variable | m5C 1 antibody light chain | WO2015075635 | 4146 |
| | | region | variable region amino acid | SEQ ID NO: 43 | |
| | | | sequence | | |
| PDLB361 | alpha synuclein | Light chain variable | Hu9E4VLv3 | WO2015075635 | 4147 |
| | | region | | SEQ ID NO: 5 | |
| PDLB362 | alpha synuclein | Light chain variable | mlH7 | WO2015075635 | 4148 |
| | | region | | SEQ ID NO: 83 | |
| PDLB363 | alpha synuclein | Light chain variable | mature mlH7 | WO2015075635 | 4149 |
| | | region | | SEQ ID NO: 87 | |
| PDLB364 | alpha synuclein | Light chain variable | BA3: 38Fl1/2_8 | WO2011104696 | 4150 |
| | protofibrils | region | | SEQ ID NO: 60 | |
| PDLB365 | alpha synuclein | Light chain variable | BA3: 38fl1/2_8 | WO2011104696 | 4151 |
| | protofibrils | region | | SEQ ID NO: 61 | |
| PDLB366 | alpha synuclein | Light chain variable | BA4: 48B11/8 | WO2011104696 | 4152 |
| | protofibrils | region | | SEQ ID NO: 62 | |
| PDLB367 | alpha synuclein | Light chain variable | BA4: 48B11/8 | WO2011104696 | 4153 |
| | protofibrils | region | | SEQ ID NO: 63 | |
| PDLB368 | amyloid | Light chain variable | F11G3 | U.S. Pat. No. 9,125,846 | 4154 |
| | oligomers | region | | SEQ ID NO: 12 | |
| PDLB369 | DR6 and P75 | Light chain variable | M73-C04 | WO2010062904 | 4155 |
| | | region | | SEQ ID NO: 102 | |
| PDLB370 | DR6 and P75 | Light chain variable | 1P1D6.3 | WO2010062904 | 4156 |
| | | region | | SEQ ID NO: 112 | |
| PDLB371 | DR6 and P75 | Light chain variable | M50-H02 | WO2010062904 | 4157 |
| | | region | | SEQ ID NO: 12 | |
| PDLB372 | DR6 and P75 | Light chain variable | 1P2F2.1 | WO2010062904 | 4158 |
| | | region | | SEQ ID NO: 122 | |
| PDLB373 | DR6 and P75 | Light chain variable | 1P5D10.2 | WO2010062904 | 4159 |
| | | region | | SEQ ID NO: 132 | |
| PDLB374 | DR6 and P75 | Light chain variable | M51-H09 | WO2010062904 | 4160 |
| | | region | | SEQ ID NO: 22 | |
| PDLB375 | DR6 and P75 | Light chain variable | M53-E04 | WO2010062904 | 4161 |
| | | region | | SEQ ID NO: 32 | |
| PDLB376 | DR6 and P75 | Light chain variable | M53-F04 | WO2010062904 | 4162 |
| | | region | | SEQ ID NO: 42 | |
| PDLB377 | DR6 and P75 | Light chain variable | M62-B02 | WO2010062904 | 4163 |
| | | region | | SEQ ID NO: 52 | |
| PDLB378 | DR6 and P75 | Light chain variable | M63-E10 | WO2010062904 | 4164 |
| | | region | | SEQ ID NO: 62 | |
| PDLB379 | DR6 and P75 | Light chain variable | M66-B03 | WO2010062904 | 4165 |
| | | region | | SEQ ID NO: 72 | |
| PDLB380 | DR6 and P75 | Light chain variable | M67-G02 | WO2010062904 | 4166 |
| | | region | | SEQ ID NO: 82 | |
| PDLB381 | DR6 and P75 | Light chain variable | M72-F03 | WO2010062904 | 4167 |
| | | region | | SEQ ID NO: 92 | |
| PDLB382 | LPG | Light chain variable | #7 | U.S. Pat. No. 8,591,902 | 4168 |
| | (lysophosphatidyl | region | | SEQ ID NO: 17 | |
| | glucoside) | | | | |
| PDLB383 | LPG | Light chain variable | #15 | U.S. Pat. No. 8,591,902 | 4169 |
| | (lysophosphatidyl | region | | SEQ ID NO: 7 | |
| | glucoside) | | | | |
| PDLB384 | MAG | Light chain variable | | U.S. Pat. No. 8,071,731 | 4170 |
| | | region | | SEQ ID NO: 16 | |
| PDLB385 | MAG | Light chain variable | | U.S. Pat. No. 8,071,731 | 4171 |
| | | region | | SEQ ID NO: 17 | |
| PDLB386 | MAG | Light chain variable | | U.S. Pat. No. 8,071,731 | 4172 |
| | | region | | SEQ ID NO: 18 | |
| PDLB387 | MAG | Light chain variable | | U.S. Pat. No. 8,071,731 | 4173 |
| | | region | | SEQ ID NO: 19 | |
| PDLB388 | MAI (myelin | Light chain variable | | WO2013158748 | 4174 |
| | associated | region | | SEQ ID NO: 11 | |
| | inhibitor) | | | | |
| PDLB389 | MAI (myelin | Light chain variable | | WO2013158748 | 4175 |
| | associated | region | | SEQ ID NO: 27 | |
| | inhibitor) | | | | |
| PDLB390 | NMDA | Light chain variable | | EP2805972 SEQ | 4176 |
| | | region | | ID NO: 44 | |
| PDLB391 | NOGO | Light chain variable | H1L6, H5L6, H6L6, | US20140147435 | 4177 |
| | | region | H14L6, H15L6, H16L6, | SEQ ID NO: 19 | |
| | | | H17L6, H18L6, H19L6, | | |
| | | | H20L6, H21L6, H22L6, | | |
| | | | H23L6, H24L6, H25L6, | | |
| | | | H700L6 | | |
| PDLB392 | NOGO | Light chain variable | H1L13, H5L13, H6L13, | US20140147435 | 4178 |
| | | region | H14L13, H15L13, | SEQ ID NO: 20 | |
| | | | H16L13, H17L13, | | |
| | | | H18L13, H19L13, | | |
| | | | H20L13, H21L13, | | |
| | | | H22L13, H23L13, | | |
| | | | H24L13, H25L13, | | |
| | | | H700L13 | | |
| PDLB393 | NOGO | Light chain variable | H1L14, H5L14, H6L14, | US20140147435 | 4179 |
| | | region | H14L14, H15L14, | SEQ ID NO: 21 | |
| | | | H16L14, H17L14, | | |
| | | | H18L14, H19L14, | | |
| | | | H20L14, H21L14, | | |
| | | | H22L14, H23L14, | | |
| | | | H24L14, H25L14, | | |
| | | | H700L14 | | |
| PDLB394 | NOGO | Light chain variable | H1L15, H5L15, H6L15, | US20140147435 | 4180 |
| | | region | H14L15, H15L15, | SEQ ID NO: 22 | |
| | | | H16L15, H17L15, | | |
| | | | H18L15, H19L15, | | |
| | | | H20L15, H21L15, | | |
| | | | H22L15, H23L15, | | |
| | | | H24L15, H25L15, | | |
| | | | H700L15 | | |
| PDLB395 | NOGO | Light chain variable | H1L16, H5L16, H6L16, | US20140147435 | 4181 |
| | | region | H14L16, H15L16, | SEQ ID NO: 23 | |
| | | | H16L16, H17L16, | | |
| | | | H18L16, H19L16, | | |
| | | | H20L16, H21L16, | | |
| | | | H22L16, H23L16, | | |
| | | | H24L16, H25L16, | | |
| | | | H700L16 | | |
| PDLB396 | NOGO | Light chain variable | H1L17, H5L17, H6L17, | US20140147435 | 4182 |
| | | region | H14L17, H15L17, | SEQ ID NO: 24 | |
| | | | H16L17, H17L17, | | |
| | | | H18L17, H19L17, | | |
| | | | H20L17, H21L17, | | |
| | | | H22L17, H23L17, | | |
| | | | H24L17, H25L17, | | |
| | | | H700L17 | | |
| PDLB397 | NOGO | Light chain variable | H1L18, H5L18, H6L18, | US20140147435 | 4183 |
| | | region | H14L18, H15L18, | SEQ ID NO: 25 | |
| | | | H16L18, H17L18, | | |
| | | | H18L18, H19L18, | | |
| | | | H20L18, H21L18, | | |
| | | | H22L18, H23L18, | | |
| | | | H24L18, H25L18, | | |
| | | | H700L18 | | |
| PDLB398 | NOGO | Light chain variable | H5L11, H6L11, H14L11, | US20140147435 | 4184 |
| | | region | H15L11, H16L11, | SEQ ID NO: 78 | |
| | | | H17L11, H18L11, | | |
| | | | H19L11, H20L11, | | |
| | | | H21L11, H22L11, | | |
| | | | H23L11, H24L11, | | |
| | | | H25L11, H700L11 | | |
| PDLB399 | Nogo-66 | Light chain variable | Antibody clone 50 | US20140065155 | 4185 |
| | | region | | SEQ ID NO: 4 | |
| PDLB400 | Nogo-66 | Light chain variable | Antibody clone 51 | US20140065155 | 4186 |
| | | region | | SEQ ID NO: 6 | |
| PDLB401 | NogoA/NiG | Light chain variable | 6A3-Ig4 | WO2009056509 | 4187 |
| | | region | | SEQ ID NO: 25 | |
| PDLB402 | NogoA/NiG | Light chain variable | 6A3-IgG1 | WO2009056509 | 4188 |
| | | region | | SEQ ID NO: 5 | |
| PDLB403 | RGM A | Light chain variable | 5F9.1-GL, 5F9.1-GL, | US20150183871 | 4189 |
| | | region | 5F9.1-GL, 5F9.1-GL, | SEQ ID NO: 44 | |
| | | | 5F9.1-GL, 5F9.1-GL, | | |
| | | | 5F9.1-GL, 5F9.1-GL, | | |
| | | | 5F9.1-GL, 5F9.1-GL, | | |
| | | | h5F9.4, h5F9.11, h5F9.12 | | |
| PDLB404 | RGM A | Light chain variable | 5F9.2-GL, 5F9.2-GL, | US20150183871 | 4190 |
| | | region | 5F9.2-GL, 5F9.2-GL, | SEQ ID NO: 45 | |
| | | | 5F9.2-GL, 5F9.2-GL, | | |
| | | | 5F9.2-GL, 5F9.2-GL, | | |
| | | | 5F9.2-GL, 5F9.2-GL, | | |
| | | | h5F9.5, h5F9.19, h5F9.20 | | |
| PDLB405 | RGM A | Light chain variable | 5F9.3-GL, 5F9.3-GL, | US20150183871 | 4191 |
| | | region | 5F9.3-GL, 5F9.3-GL, | SEQ ID NO: 46 | |
| | | | 5F9.3-GL, 5F9.3-GL, | | |
| | | | 5F9.3-GL, 5F9.3-GL, | | |
| | | | 5F9.3-GL, 5F9.3-GL, | | |
| | | | h5F9.6, h5F9.21, h5F9.22 | | |
| PDLB406 | RGM A | Light chain variable | h5F9.5, h5F9.6, h5F9.7, | US20150183871 | 4192 |
| | | region | h5F9.8, h5F9.9, h5F9.10 | SEQ ID NO: 48 | |
| PDLB407 | RGM A | Light chain variable | h5F9.11, h5F9.19, h5F9.21 | US20150183871 | 4193 |
| | | region | | SEQ ID NO: 49 | |
| PDLB408 | RGM A | Light chain variable | h5F9.12, h5F9.20, | US20150183871 | 4194 |
| | | region | h5F9.22, h5F9.23, | SEQ ID NO: 50 | |
| | | | h5F9.25, h5F9.25, h5F9.26 | | |
| PDLB409 | RGM A | Light chain variable | h5F9.1, h5F9.7, h5F9.23 | US20150183871 | 4195 |
| | | region | | SEQ ID NO: 51 | |
| PDLB410 | RGM A | Light chain variable | h5F9.2, h5F9.8, h5F9.25 | US20150183871 | 4196 |
| | | region | | SEQ ID NO: 52 | |
| PDLB411 | RGMa | Light chain variable | AE12-15 | US20140023659 | 4197 |
| | | region | | SEQ ID NO: 103 | |
| PDLB412 | RGMa | Light chain variable | AE12-20 | US20140023659 | 4198 |
| | | region | | SEQ ID NO: 111 | |
| PDLB413 | RGMa | Light chain variable | AE12-21 | US20140023659 | 4199 |
| | | region | | SEQ ID NO: 119 | |
| PDLB414 | RGMa | Light chain variable | AE12-23 | US20140023659 | 4200 |
| | | region | | SEQ ID NO: 127 | |
| PDLB415 | RGMa | Light chain variable | AE12-2 | US20140023659 | 4201 |
| | | region | | SEQ ID NO: 13 | |
| PDLB416 | RGMa | Light chain variable | AE12-24 | US20140023659 | 4202 |
| | | region | | SEQ ID NO: 135 | |
| PDLB417 | RGMa | Light chain variable | AE12-3 | US20140023659 | 4203 |
| | | region | | SEQ ID NO: 21 | |
| PDLB418 | RGMa | Light chain variable | AE12-4 | US20140023659 | 4204 |
| | | region | | SEQ ID NO: 29 | |
| PDLB419 | RGMa | Light chain variable | AE12-5 | US20140023659 | 4205 |
| | | region | | SEQ ID NO: 37 | |
| PDLB420 | RGMa | Light chain variable | AE12-6 | US20140023659 | 4206 |
| | | region | | SEQ ID NO: 45 | |
| PDLB421 | RGMa | Light chain variable | AE12-1 | US20140023659 | 4207 |
| | | region | | SEQ ID NO: 5 | |
| PDLB422 | RGMa | Light chain variable | AE12-7 | US20140023659 | 4208 |
| | | region | | SEQ ID NO: 53 | |
| PDLB423 | RGMa | Light chain variable | AE12-8 | US20140023659 | 4209 |
| | | region | | SEQ ID NO: 61 | |
| PDLB424 | RGMa | Light chain variable | AE12-13 | US20140023659 | 4210 |
| | | region | | SEQ ID NO: 95 | |
| PDLB425 | α-synuclein | Light chain variable | Syn-01 | WO2014132210 | 4211 |
| | aggregates | region | | SEQ ID NO: 12 | |
| PDLB426 | α-synuclein | Light chain variable | Syn-F1 | WO2014132210 | 4212 |
| | aggregates | region | | SEQ ID NO: 4 | |
| PDLB427 | α-synuclein | Light chain variable | Syn-F2 | WO2014132210 | 4213 |
| | aggregates | region | | SEQ ID NO: 8 | |
| PDLB428 | NOGO | Light chain variable | 2A10 construct | WO2007003421 | 4214 |
| | | region humanized | | SEQ ID NO: 78 | |
| | | construct L11 | | | |
| PDLB429 | NOGO | Light chain variable | 2A10 construct | WO2007003421 | 4215 |
| | | region humanized | | SEQ ID NO: 20 | |
| | | construct L13 | | | |
| PDLB430 | NOGO | Light chain variable | 2A10 construct | WO2007003421 | 4216 |
| | | region humanized | | SEQ ID NO: 21 | |
| | | construct L14 | | | |
| PDLB431 | NOGO | Light chain variable | 2A10 construct | WO2007003421 | 4217 |
| | | region humanized | | SEQ ID NO: 22 | |
| | | construct L15 | | | |
| PDLB432 | NOGO | Light chain variable | 2A10 construct | WO2007003421 | 4218 |
| | | region humanized | | SEQ ID NO: 23 | |
| | | construct L16 | | | |
| PDLB433 | NOGO | Light chain variable | 2A10 construct | WO2007003421 | 4219 |
| | | region humanized | | SEQ ID NO: 24 | |
| | | construct L17 | | | |
| PDLB434 | NOGO | Light chain variable | 2A10 construct | WO2007003421 | 4220 |
| | | region humanized | | SEQ ID NO: 25 | |
| | | construct L18 | | | |
| PDLB435 | NOGO | Light chain variable | 2A10 construct | WO2007003421 | 4221 |
| | | region humanized | | SEQ ID NO: 19 | |
| | | construct L16 | | | |
| PDLB436 | alpha synuclein | Light chain version | Hu1H7 | U.S. Pat. No. 8,790,644 | 4222 |
| | | 3 (variable | | SEQ ID NO: 53 | |
| | | region + constant | | | |
| | | region with | | | |
| | | arginine) | | | |
| PDLB437 | alpha synuclein | Light chain version | Hu1H7 | U.S. Pat. No. 8,790,644 | 4223 |
| | | 3 (variable | | SEQ ID NO: 54 | |
| | | region + constant | | | |
| | | region without | | | |
| | | arginine) | | | |

In some embodiments, the payload region of the viral particle comprises one or more nucleic acid sequences encoding one or more of the polypeptides that comprise a portion of filamentous bacteriophage gene 3 protein (g3p) sufficient to bind to and/or disaggregate amyloid described in International Publication No. WO2014193935, the contents of which are herein incorporated by reference in their entirety. As a non-limiting example, the payload region of the viral particle comprises one or more nucleic acid sequences encoding one or more of the polypeptides described in WO2014193935 may be used to treat, prevent and/or reduce the effects of Parkinson's Disease and/or dementia. As another non-limiting example, the payload region of the viral particle comprises one or more nucleic acid sequences encoding one or more of the polypeptides described in WO2014193935 may be used to treat, prevent and/or reduce the effects of Alzheimer's Disease. As another non-limiting example, the payload region of the viral particle comprises one or more nucleic acid sequences encoding one or more of the polypeptides described in WO2014193935 may be used to treat, prevent and/or reduce the effects of Huntington's Disease. As another non-limiting example, the payload region of the viral particle comprises one or more nucleic acid sequences encoding one or more of the polypeptides described in WO2014193935 may be used to treat, prevent and/or reduce the effects of muscle disease such as, but not limited to, Multiple System Atrophy (MSA), Amyotrophic Lateral Sclerosis (ALS) and Duchenne Muscular Dystrophy (DMD).

### Alzheimer's Disease Antibodies

In some embodiments, the payload region of the viral particle comprises one or more nucleic acid sequences encoding one or more of the Alzheimer's Disease payload antibody polypeptides listed in Table 4 (AD1-AD1178; SEQ ID NO: 4224-5401).

TABLE 4
Alzheimer's Disease Antibodies

| Antibody No. | Target | Description | Antibody Name | Reference Information | SEQ ID NO |
|---|---|---|---|---|---|
| AD1 | amyloid | consensus | M13 g3p, fd g3p, fl | US20150376239 | 4224 |
| | proteins | sequence | g3p | SEQ ID NO: 4 | |
| AD2 | amyloid | consensus | 12-2 g3p, Ike g3p | US20150376239 | 4225 |
| | proteins | sequence | | SEQ ID NO: 7 | |
| AD3 | Aβ amyloid | Consensus | | WO2006066049 | 4226 |
| | | sequence for kappa | | SEQ ID NO: 14 | |
| | | chain | | | |
| AD4 | Aβ amyloid | Consensus | | WO2006066049 | 4227 |
| | | sequence for kappa | | SEQ ID NO: 15 | |
| | | chain | | | |
| AD5 | Aβ amyloid | Consensus | | WO2006066049 | 4228 |
| | | sequence for kappa | | SEQ ID NO: 16 | |
| | | chain | | | |
| AD6 | Aβ amyloid | Consensus | | WO2006066049 | 4229 |
| | | sequence for kappa | | SEQ ID NO: 17 | |
| | | chain | | | |
| AD7 | Aβ amyloid | Consensus | | WO2006066049 | 4230 |
| | | sequence for | | SEQ ID NO: 18 | |
| | | lambda chain | | | |
| AD8 | Aβ amyloid | Consensus | | WO2006066049 | 4231 |
| | | sequence for | | SEQ ID NO: 19 | |
| | | lambda chain | | | |
| AD9 | Aβ amyloid | Consensus | | WO2006066049 | 4232 |
| | | sequence for | | SEQ ID NO: 20 | |
| | | lambda chain | | | |
| AD10 | 118-126 of α- | constant region | IgG1 | US20150259404 | 4233 |
| | synuclein | | | SEQ ID NO: 38 | |
| AD11 | beta A4 | Fc region | Antibody A | WO2007068429 | 4234 |
| | peptide/Alpha | | | SEQ ID NO: 6 | |
| | beta 5 | | | | |
| AD12 | amyloid | Fusion protein | M13 g3p | US20150376239 | 4235 |
| | proteins | | | SEQ ID NO: 1 | |
| AD13 | amyloid | Fusion protein | Construct 5 | US20150376239 | 4236 |
| | proteins | | | SEQ ID NO: 11 | |
| AD14 | amyloid | Fusion protein | Construct 6 | US20150376239 | 4237 |
| | proteins | | | SEQ ID NO: 13 | |
| AD15 | amyloid | Fusion protein | fd N2 | US20150376239 | 4238 |
| | proteins | | | SEQ ID NO: 14 | |
| AD16 | amyloid | Fusion protein | f1 N2 | US20150376239 | 4239 |
| | proteins | | | SEQ ID NO: 15 | |
| AD17 | amyloid | Fusion protein | M13 N2 | US20150376239 | 4240 |
| | proteins | | | SEQ ID NO: 16 | |
| AD18 | amyloid | Fusion protein | Ike N2 | US20150376239 | 4241 |
| | proteins | | | SEQ ID NO: 17 | |
| AD19 | amyloid | Fusion protein | 12-2 N2 | US20150376239 | 4242 |
| | proteins | | | SEQ ID NO: 18 | |
| AD20 | amyloid | Fusion protein | If1 N2 | US20150376239 | 4243 |
| | proteins | | | SEQ ID NO: 19 | |
| AD21 | amyloid | Fusion protein | fd g3p | US20150376239 | 4244 |
| | proteins | | | SEQ ID NO: 2 | |
| AD22 | amyloid | Fusion protein | Construct 3 | US20150376239 | 4245 |
| | proteins | | | SEQ ID NO: 20 | |
| AD23 | amyloid | Fusion protein | Construct 3m g3p | US20150376239 | 4246 |
| | proteins | | portion | SEQ ID NO: 24 | |
| AD24 | amyloid | Fusion protein | If1 g3p | US20150376239 | 4247 |
| | proteins | | | SEQ ID NO: 29 | |
| AD25 | amyloid | Fusion protein | f1 g3p | US20150376239 | 4248 |
| | proteins | | | SEQ ID NO: 3 | |
| AD26 | amyloid | Fusion protein | fd g3p | US20150376239 | 4249 |
| | proteins | | | SEQ ID NO: 30 | |
| AD27 | amyloid | Fusion protein | Construct 8, rs-g3p | US20150376239 | 4250 |
| | proteins | | (If1-N1N2)-hlgG1-Fc | SEQ ID NO: 31 | |
| AD28 | amyloid | Fusion protein | I2-2 g3p | US20150376239 | 4251 |
| | proteins | | | SEQ ID NO: 5 | |
| AD29 | amyloid | Fusion protein | Ike g3p | US20150376239 | 4252 |
| | proteins | | | SEQ ID NO: 6 | |
| AD30 | amyloid | Fusion protein | If1 g3p | US20150376239 | 4253 |
| | proteins | | | SEQ ID NO: 8 | |
| AD31 | amyloid | Fusion protein | Construct 4 | US20150376239 | 4254 |
| | proteins | | | SEQ ID NO: 9 | |
| AD32 | ACTH | Heavy chain | Ab4 | WO2015127288 | 4255 |
| | | | | SEQ ID NO: 121 | |
| AD33 | ACTH | Heavy chain | Ab5 | WO2015127288 | 4256 |
| | | | | SEQ ID NO: 161 | |
| AD34 | ACTH | Heavy chain | Ab6 | WO2015127288 | 4257 |
| | | | | SEQ ID NO: 201 | |
| AD35 | ACTH | Heavy chain | Ab7 | WO2015127288 | 4258 |
| | | | | SEQ ID NO: 241 | |
| AD36 | ACTH | Heavy chain | Ab9 | WO2015127288 | 4259 |
| | | | | SEQ ID NO: 281 | |
| AD37 | ACTH | Heavy chain | Ab10 | WO2015127288 | 4260 |
| | | | | SEQ ID NO: 321 | |
| AD38 | ACTH | Heavy chain | Ab11 | WO2015127288 | 4261 |
| | | | | SEQ ID NO: 361 | |
| AD39 | ACTH | Heavy chain | Ab12 | WO2015127288 | 4262 |
| | | | | SEQ ID NO: 401 | |
| AD40 | ACTH | Heavy chain | Ab2 | WO2015127288 | 4263 |
| | | | | SEQ ID NO: 41 | |
| AD41 | ACTH | Heavy chain | Ab1.H | WO2015127288 | 4264 |
| | | | | SEQ ID NO: 441 | |
| AD42 | ACTH | Heavy chain | Ab2.H | WO2015127288 | 4265 |
| | | | | SEQ ID NO: 481 | |
| AD43 | ACTH | Heavy chain | Ab3.H | WO2015127288 | 4266 |
| | | | | SEQ ID NO: 521 | |
| AD44 | ACTH | Heavy chain | Ab4.H | WO2015127288 | 4267 |
| | | | | SEQ ID NO: 561 | |
| AD45 | ACTH | Heavy chain | Ab6.H | WO2015127288 | 4268 |
| | | | | SEQ ID NO: 601 | |
| AD46 | ACTH | Heavy chain | Ab7.H | WO2015127288 | 4269 |
| | | | | SEQ ID NO: 641 | |
| AD47 | ACTH | Heavy chain | Ab7A.H | WO2015127288 | 4270 |
| | | | | SEQ ID NO: 681 | |
| AD48 | ACTH | Heavy chain | Ab10.H | WO2015127288 | 4271 |
| | | | | SEQ ID NO: 721 | |
| AD49 | ACTH | Heavy chain | Ab11.H | WO2015127288 | 4272 |
| | | | | SEQ ID NO: 761 | |
| AD50 | ACTH | Heavy chain | Ab11A.H | WO2015127288 | 4273 |
| | | | | SEQ ID NO: 801 | |
| AD51 | ACTH | Heavy chain | Ab3 | WO2015127288 | 4274 |
| | | | | SEQ ID NO: 81 | |
| AD52 | ACTH | Heavy chain | Ab12.H | WO2015127288 | 4275 |
| | | | | SEQ ID NO: 841 | |
| AD53 | ACTH | Heavy chain | Ab1 | WO2015127288 | 4276 |
| | | | | SEQ ID NO: 1 | |
| AD54 | Alpha beta | Heavy chain | Gantenerumab | Immunogenetics | 4277 |
| | fibril | | | Information | |
| | | | | System; CHAIN | |
| | | | | ID NO: 8894_H. | |
| AD55 | amyloid beta | Heavy chain | | U.S. Pat. No. 719,576 | 4278 |
| | peptide Aβ | | | SEQ ID NO: 12 | |
| AD56 | Amyloid | Heavy chain | 2 Fab of Yw412.8.31 | Wang, W. et al “A | 4279 |
| | beta/BACE1 | | | Therapeutic | |
| | | | | Antibody Targeting | |
| | | | | BACE1 Inhibits | |
| | | | | Amyloid. NO:- | |
| | | | | {beta}Production | |
| | | | | in Vivo” Sci Transl | |
| | | | | Med 3 (84), | |
| | | | | 84RA43 (2011), | |
| | | | | NCBI Accession # | |
| | | | | 3RIG_H (222aa) | |
| AD57 | amyloid or | Heavy chain | Humanized C2 | WO2008061796 | 4280 |
| | amyloid-like | | | SEQ ID NO: 4 | |
| | proteins | | | | |
| AD58 | amyloid protein | Heavy chain | C2 | US20100150906 | 4281 |
| | | | | SEQ ID NO: 16 | |
| AD59 | amyloids | Heavy chain | #118 | WO2010012004 | 4282 |
| | | | | SEQ ID NO: 11 | |
| AD60 | amyloids | Heavy chain | #121 | WO2010012004 | 4283 |
| | | | | SEQ ID NO: 13 | |
| AD61 | amyloids | Heavy chain | #204 | WO2010012004 | 4284 |
| | | | | SEQ ID NO: 16 | |
| AD62 | amyloids | Heavy chain | #205 | WO2010012004 | 4285 |
| | | | | SEQ ID NO: 18 | |
| AD63 | APP | Heavy chain | F5.100 | WO2014151747 | 4286 |
| | | | | SEQ ID NO: 2 | |
| AD64 | APP | Heavy chain | BBSl MAb | WO2014151747 | 4287 |
| | | | | SEQ ID NO: 24 | |
| AD65 | APP | Heavy chain | F5.87 | WO2014151747 | 4288 |
| | | | | SEQ ID NO: 26 | |
| AD66 | APP | Heavy chain | F5.87 | WO2014151747 | 4289 |
| | | | | SEQ ID NO: 52 | |
| AD67 | Aβ amyloids | Heavy chain | Humanized 12A11, | U.S. Pat. No. 8,784,810 | 4290 |
| | | | version 3 | SEQ ID NO: 11 | |
| AD68 | Aβ amyloids | Heavy chain | Humanized 12A11, | U.S. Pat. No. 8,784,810 | 4291 |
| | | | version 4.1 | SEQ ID NO: 12 | |
| AD69 | Aβ amyloids | Heavy chain | Humanized 12A11, | U.S. Pat. No. 8,784,810 | 4292 |
| | | | version 4.2 | SEQ ID NO: 13 | |
| AD70 | Aβ amyloids | Heavy chain | Humanized 12A11, | U.S. Pat. No. 8,784,810 | 4293 |
| | | | version 4.3 | SEQ ID NO: 14 | |
| AD71 | Aβ amyloids | Heavy chain | Humanized 12A11, | U.S. Pat. No. 8,784,810 | 4294 |
| | | | version 4.4 | SEQ ID NO: 15 | |
| AD72 | Aβ amyloids | Heavy chain | Humanized 12A11, | U.S. Pat. No. 8,784,810 | 4295 |
| | | | version 5.1 | SEQ ID NO: 16 | |
| AD73 | Aβ amyloids | Heavy chain | Humanized 12A11, | U.S. Pat. No. 8,784,810 | 4296 |
| | | | version 5.2 | SEQ ID NO: 17 | |
| AD74 | Aβ amyloids | Heavy chain | Humanized 12A11, | U.S. Pat. No. 8,784,810 | 4297 |
| | | | version 5.3 | SEQ ID NO: 18 | |
| AD75 | Aβ amyloids | Heavy chain | Humanized 12A11, | U.S. Pat. No. 8,784,810 | 4298 |
| | | | version 5.4 | SEQ ID NO: 19 | |
| AD76 | Aβ amyloids | Heavy chain | Humanized 12A11, | U.S. Pat. No. 8,784,810 | 4299 |
| | | | version 5.5 | SEQ ID NO: 20 | |
| AD77 | Aβ amyloids | Heavy chain | Humanized 12A11, | U.S. Pat. No. 8,784,810 | 4300 |
| | | | version 5.6 | SEQ ID NO: 21 | |
| AD78 | Aβ amyloids | Heavy chain | Humanized 12A11, | U.S. Pat. No. 8,784,810 | 4301 |
| | | | version 6.1 | SEQ ID NO: 22 | |
| AD79 | Aβ amyloids | Heavy chain | Humanized 12A11, | U.S. Pat. No. 8,784,810 | 4302 |
| | | | version 6.2 | SEQ ID NO: 23 | |
| AD80 | Aβ amyloids | Heavy chain | Humanized 12A11, | U.S. Pat. No. 8,784,810 | 4303 |
| | | | version 6.3 | SEQ ID NO: 24 | |
| AD81 | Aβ amyloids | Heavy chain | Humanized 12A11, | U.S. Pat. No. 8,784,810 | 4304 |
| | | | version 6.4 | SEQ ID NO: 25 | |
| AD82 | Aβ amyloids | Heavy chain | Humanized 12A11, | U.S. Pat. No. 8,784,810 | 4305 |
| | | | version 7 | SEQ ID NO: 26 | |
| AD83 | Aβ amyloids | Heavy chain | Humanized 12A11, | U.S. Pat. No. 8,784,810 | 4306 |
| | | | version 8 | SEQ ID NO: 27 | |
| AD84 | Aβ amyloids | Heavy chain | Humanized 3D6 | U.S. Pat. No. 8,784,810 | 4307 |
| | | | (Bapineuzumab), | SEQ ID NO: 5 | |
| | | | version 3 | | |
| AD85 | Aβ amyloids | Heavy chain | Humanized 12A11, | U.S. Pat. No. 8,784,810 | 4308 |
| | | | version 2 | SEQ ID NO: 9 | |
| AD86 | beta amyloid | Heavy chain | | US10476265 | 4309 |
| | | | | SEQ ID NO: 20 | |
| AD87 | beta amyloid | Heavy chain | (13C3) | US13319710 | 4310 |
| | | | | SEQ ID NO: 2 | |
| AD88 | beta amyloid | Heavy chain | | US13319710 | 4311 |
| | | | | SEQ ID NO: 26 | |
| AD89 | beta amyloid | Heavy chain | C2 | US20070166311 | 4312 |
| | | | | SEQ ID NO: 22 | |
| AD90 | beta amyloid | Heavy chain | Solanezumab | Immunogenetics | 4313 |
| | peptide | | | Information | |
| | | | | System; CHAIN | |
| | | | | ID NO: 9097_H. | |
| AD91 | beta amyloid | Heavy chain | Mature H1 | WO2007113172 | 4314 |
| | peptide | | | SEQ ID NO: 34 | |
| AD92 | beta amyloid | Heavy chain | Mature H3 | WO2007113172 | 4315 |
| | peptide | | | SEQ ID NO: 38 | |
| AD93 | beta-amyloid | Heavy chain | Aducanumab, | | 4316 |
| | | | BIIB0307 | | |
| AD94 | EAG1 | Heavy chain | chimeric ImAb3 | WO2006037604 | 4317 |
| | | | | SEQ ID NO: 12 | |
| AD95 | EAG1 | Heavy chain | chimeric ImAb4 | WO2006037604 | 4318 |
| | | | | SEQ ID NO: 16 | |
| AD96 | EAG1 | Heavy chain | HC-lmAb3-humVH3- | WO2006037604 | 4319 |
| | | | 72 | SEQ ID NO: 20 | |
| AD97 | EAG1 | Heavy chain | HC-lmAb4-humVH4- | WO2006037604 | 4320 |
| | | | 59 | SEQ ID NO: 24 | |
| AD98 | EAG1 | Heavy chain | HC-lmAb3-humVH3 | WO2006037604 | 4321 |
| | | | 23 | SEQ ID NO: 28 | |
| AD99 | EAG1 | Heavy chain | HC-lmAb3-humVH2 | WO2006037604 | 4322 |
| | | | 26 | SEQ ID NO: 32 | |
| AD100 | EAG1 | Heavy chain | HC-lmAb4-humVH1-3 | WO2006037604 | 4323 |
| | | | | SEQ ID NO: 36 | |
| AD101 | EAG1 | Heavy chain | ImAb4 | WO2006037604 | 4324 |
| | | | | SEQ ID NO: 4 | |
| AD102 | EAG1 | Heavy chain | ImAb3 | WO2006037604 | 4325 |
| | | | | SEQ ID NO: 8 | |
| AD103 | human beta- | Heavy chain | Ponezumab, PF- | U.S. Pat. No. 7,807,165 | 4326 |
| | amyloid | | 04360365, RN-1219, | SEQ ID NO: 11 | |
| | | | clone 9TL | | |
| AD104 | IGG1 Abeta | Heavy chain | Humanized C2 | US20090155249 | 4327 |
| | | | | SEQ ID NO: 16 | |
| AD105 | NOGO | Heavy chain | H6L13 FL | US20140147435 | 4328 |
| | | | | SEQ ID NO: 27 | |
| AD106 | NOGO | Heavy chain | H16L16 FL, H16L18 | US20140147435 | 4329 |
| | | | FL | SEQ ID NO: 31 | |
| AD107 | NOGO | Heavy chain | H18L16 FL | US20140147435 | 4330 |
| | | | | SEQ ID NO: 33 | |
| AD108 | NOGO | Heavy chain | H19L13 FL, H19L16 | US20140147435 | 4331 |
| | | | FL, H19L18 FL | SEQ ID NO: 92 | |
| AD109 | NOGO | Heavy chain | H20L13 FL, H20L16 | US20140147435 | 4332 |
| | | | FL, H20L18 FL | SEQ ID NO: 93 | |
| AD110 | NOGO | Heavy chain | H21L13 EL, H21L16 | US20140147435 | 4333 |
| | | | FL, H21L18 FL | SEQ ID NO: 94 | |
| AD111 | NOGO | Heavy chain | H25L13 FL, H25L16 | US20140147435 | 4334 |
| | | | FL, H25L18 FL | SEQ ID NO: 98 | |
| AD112 | Nogo receptor-1 | Heavy chain | 5B10 | US20090215691 | 4335 |
| | | | | SEQ ID NO: 16 | |
| AD113 | Nogo receptor-1 | Heavy chain | 5B10 | US20090215691 | 4336 |
| | | | | SEQ ID NO: 18 | |
| AD114 | PrPC and/or | Heavy chain | | US20150166668 | 4337 |
| | PrPSc | | | SEQ ID NO: 10 | |
| AD115 | PrPC and/or | Heavy chain | | U.S. Pat. No. 8,852,587 | 4338 |
| | PrPSc | | | SEQ ID NO: 4 | |
| AD116 | tau | Heavy chain | VH antibody | US20150252102 | 4339 |
| | | | | SEQ ID NO: 93 | |
| AD117 | tau | Heavy chain | hACl-36-3A8 Ab1 | WO2013151762 | 4340 |
| | | | | SEQ ID NO: 24 | |
| AD118 | tau | Heavy chain | hACl-36-3B8 Ab1 | WO2013151762 | 4341 |
| | | | | SEQ ID NO: 25 | |
| AD119 | tau | Heavy chain | hACl-36-3A8 Ab1.v2 | WO2013151762 | 4342 |
| | | | | SEQ ID NO: 26 | |
| AD120 | tau | Heavy chain | hACl-36-3A8 Ab1.v3 | WO2013151762 | 4343 |
| | | | | SEQ ID NO: 27 | |
| AD121 | tau | Heavy chain | hACl-36-3A8 Ab1.v4 | WO2013151762 | 4344 |
| | | | | SEQ ID NO: 28 | |
| AD122 | tau | Heavy chain | hACl-36-3B8 Ab1.v2 | WO2013151762 | 4345 |
| | | | | SEQ ID NO: 29 | |
| AD123 | tau | Heavy chain | hACl-36-3B8 Ab1.v3 | WO2013151762 | 4346 |
| | | | | SEQ ID NO: 30 | |
| AD124 | tau | Heavy chain | hACl-36-3B8 Ab1.v4 | WO2013151762 | 4347 |
| | | | | SEQ ID NO: 31 | |
| AD125 | tau | Heavy chain | IPN001 | U.S. Pat. No. 8,980,271 | 4348 |
| | | | | SEQ ID NO: 14 | |
| AD126 | tau | Heavy chain | IPN002 | U.S. Pat. No. 8,980,271 | 4349 |
| | | | | SEQ ID NO: 16 | |
| AD127 | tau | Heavy chain | ACl-36-3A8-Ab1 and | US20150175682 | 4350 |
| | | | hACl-36-2B6-Ab1 | SEQ ID NO: 16 | |
| AD128 | tau | Heavy chain | hACl-36-3A8-Ab1 | US20150175682 | 4351 |
| | | | and hACl-36-2B6-Ab1 | SEQ ID NO: 17 | |
| AD129 | tau | Heavy chain | hACl-36-2B6-Ab1 | US20150175682 | 4352 |
| | | | (IgG4) | SEQ ID NO: 25 | |
| AD130 | tau | Heavy chain | hACl-36-3A8-Ab1.v2 | US20150175682 | 4353 |
| | | | (IgG4) | SEQ ID NO: 26 | |
| AD131 | tau | Heavy chain | hACl-36-3A8-Ab1.v3 | US20150175682 | 4354 |
| | | | (IgG1) | SEQ ID NO: 27 | |
| AD132 | tau | Heavy chain | hACl-36-3A8-Ab1.v4 | US20150175682 | 4355 |
| | | | (IgG1 N297G) | SEQ ID NO: 28 | |
| AD133 | tau | Heavy chain | hACl-36-2B6-Ab1.v2 | US20150175682 | 4356 |
| | | | (IgG4) | SEQ ID NO: 29 | |
| AD134 | tau | Heavy chain | hACl-36-2B6-Ab1.v3 | US20150175682 | 4357 |
| | | | (IgG1) | SEQ ID NO: 30 | |
| AD135 | tau | Heavy chain | hACl-36-2B6-Ab1.v4 | US20150175682 | 4358 |
| | | | (IgG1 N297G) | SEQ ID NO: 31 | |
| AD136 | TrkA | Heavy chain | BXhVH1 | WO2009098238 | 4359 |
| | | | | SEQ ID NO: 1 | |
| AD137 | TrkA | Heavy chain | mVHEP | WO2009098238 | 4360 |
| | | | | SEQ ID NO: 15 | |
| AD138 | TrkA | Heavy chain | BXhVH2 | WO2009098238 | 4361 |
| | | | | SEQ ID NO: 2 | |
| AD139 | TrkA | Heavy chain | BXhVH3 | WO2009098238 | 4362 |
| | | | | SEQ ID NO: 3 | |
| AD140 | TrkA | Heavy chain | BXhVH4 | WO2009098238 | 4363 |
| | | | | SEQ ID NO: 4 | |
| AD141 | TrkA | Heavy chain | BXhVH5 | WO2009098238 | 4364 |
| | | | | SEQ ID NO: 5 | |
| AD142 | TrkA | Heavy chain | HUVHWOV | WO2009098238 | 4365 |
| | | | | SEQ ID NO: 6 | |
| AD143 | trk-C (NT-3 | Heavy chain | 2250 | U.S. Pat. No. 7,615,383 | 4366 |
| | trkC ligand) | | | SEQ ID NO: 42 | |
| AD144 | trk-C (NT-3 | Heavy chain | 2253 | U.S. Pat. No. 7,615,383 | 4367 |
| | trkC ligand) | | | SEQ ID NO: 43 | |
| AD145 | trk-C (NT-3 | Heavy chain | 2256 | U.S. Pat. No. 7,615,383 | 4368 |
| | trkC ligand) | | | SEQ ID NO: 44 | |
| AD146 | trk-C (NT-3 | Heavy chain | 6.1.2 | U.S. Pat. No. 7,615,383 | 4369 |
| | trkC ligand) | | | SEQ ID NO: 45 | |
| AD147 | trk-C (NT-3 | Heavy chain | 6.4.1 | U.S. Pat. No. 7,615,383 | 4370 |
| | trkC ligand) | | | SEQ ID NO: 46 | |
| AD148 | trk-C (NT-3 | Heavy chain | 2345 | U.S. Pat. No. 7,615,383 | 4371 |
| | trkC ligand) | | | SEQ ID NO: 47 | |
| AD149 | trk-C (NT-3 | Heavy chain | 2349 | U.S. Pat. No. 7,615,383 | 4372 |
| | trkC ligand) | | | SEQ ID NO: 48 | |
| AD150 | | Heavy chain | Crenezuma heavy | | 4373 |
| | | | CHAIN | | |
| AD151 | | Heavy chain | Gantenerumab heavy | | 4374 |
| | | | chain | | |
| AD152 | | Heavy chain | Ponezumab heavy | | 4375 |
| | | | CHAIN | | |
| AD153 | | Heavy chain | Solanezumab heavy | | 4376 |
| | | | CHAIN | | |
| AD154 | Aβ amyloid | Heavy chain | | WO2006066049 | 4377 |
| | | consensus | | SEQ ID NO: 21 | |
| | | sequence | | | |
| AD155 | Aβ amyloid | Heavy chain | | WO2006066049 | 4378 |
| | | consensus | | SEQ ID NO: 22 | |
| | | sequence | | | |
| AD156 | Aβ amyloid | Heavy chain | | WO2006066049 | 4379 |
| | | consensus | | SEQ ID NO: 23 | |
| | | sequence | | | |
| AD157 | Aβ amyloid | Heavy chain | | WO2006066049 | 4380 |
| | | consensus | | SEQ ID NO: 24 | |
| | | sequence | | | |
| AD158 | Aβ amyloid | Heavy chain | | WO2006066049 | 4381 |
| | | consensus | | SEQ ID NO: 25 | |
| | | sequence | | | |
| AD159 | Aβ amyloid | Heavy chain | | WO2006066049 | 4382 |
| | | consensus | | SEQ ID NO: 26 | |
| | | sequence | | | |
| AD160 | Aβ amyloid | Heavy chain | | WO2006066049 | 4383 |
| | | consensus | | SEQ ID NO: 27 | |
| | | sequence | | | |
| AD161 | BACE1 | Heavy chain | Nanobody B1 | WO2009121948 | 4384 |
| | | variable | | SEQ ID NO: 1 | |
| | | (nanobody) | | | |
| AD162 | BACE10 | Heavy chain | Nanobody B15 | WO2009121948 | 4385 |
| | | variable | | SEQ ID NO: 10 | |
| | | (nanobody) | | | |
| AD163 | BACE11 | Heavy chain | Nanobody B16 | WO2009121948 | 4386 |
| | | variable | | SEQ ID NO: 11 | |
| | | (nanobody) | | | |
| AD164 | BACE12 | Heavy chain | Nanobody B21 | WO2009121948 | 4387 |
| | | variable | | SEQ ID NO: 12 | |
| | | (nanobody) | | | |
| AD165 | BACE13 | Heavy chain | Nanobody B25 | WO2009121948 | 4388 |
| | | variable | | SEQ ID NO: 13 | |
| | | (nanobody) | | | |
| AD166 | BACE14 | Heavy chain | Nanobody B26 | WO2009121948 | 4389 |
| | | variable | | SEQ ID NO: 14 | |
| | | (nanobody) | | | |
| AD167 | BACE15 | Heavy chain | Nanobody 1B3 | WO2009121948 | 4390 |
| | | variable | | SEQ ID NO: 15 | |
| | | (nanobody) | | | |
| AD168 | BACE16 | Heavy chain | Nanobody 10C2 | WO2009121948 | 4391 |
| | | variable | | SEQ ID NO: 16 | |
| | | (nanobody) | | | |
| AD169 | BACE17 | Heavy chain | Nanobody 12B6 | WO2009121948 | 4392 |
| | | variable | | SEQ ID NO: 17 | |
| | | (nanobody) | | | |
| AD170 | BACE18 | Heavy chain | Nanobody 10B5 | WO2009121948 | 4393 |
| | | variable | | SEQ ID NO: 18 | |
| | | (nanobody) | | | |
| AD171 | BACE19 | Heavy chain | Nanobody 13A5 | WO2009121948 | 4394 |
| | | variable | | SEQ ID NO: 19 | |
| | | (nanobody) | | | |
| AD172 | BACE2 | Heavy chain | Nanobody B2 | WO2009121948 | 4395 |
| | | variable | | SEQ ID NO: 2 | |
| | | (nanobody) | | | |
| AD173 | BACE20 | Heavy chain | Nanobody 2C6 | WO2009121948 | 4396 |
| | | variable | | SEQ ID NO: 20 | |
| | | (nanobody) | | | |
| AD174 | BACE21 | Heavy chain | Nanobody 6A4 | WO2009121948 | 4397 |
| | | variable | | SEQ ID NO: 21 | |
| | | (nanobody) | | | |
| AD175 | BACE22 | Heavy chain | Nanobody 10C4 | WO2009121948 | 4398 |
| | | variable | | SEQ ID NO: 22 | |
| | | (nanobody) | | | |
| AD176 | BACE23 | Heavy chain | Nanobody 13B6 | WO2009121948 | 4399 |
| | | variable | | SEQ ID NO: 23 | |
| | | (nanobody) | | | |
| AD177 | BACE24 | Heavy chain | Nanobody 1A4 | WO2009121948 | 4400 |
| | | variable | | SEQ ID NO: 24 | |
| | | (nanobody) | | | |
| AD178 | BACE25 | Heavy chain | Nanobody 2B6 | WO2009121948 | 4401 |
| | | variable | | SEQ ID NO: 25 | |
| | | (nanobody) | | | |
| AD179 | BACE26 | Heavy chain | Nanobody 4A2 | WO2009121948 | 4402 |
| | | variable | | SEQ ID NO: 26 | |
| | | (nanobody) | | | |
| AD180 | BACE27 | Heavy chain | Nanobody 1 D4 | WO2009121948 | 4403 |
| | | variable | | SEQ ID NO: 27 | |
| | | (nanobody) | | | |
| AD181 | BACE28 | Heavy chain | Nanobody 9D3 | WO2009121948 | 4404 |
| | | variable | | SEQ ID NO: 28 | |
| | | (nanobody) | | | |
| AD182 | BACE3 | Heavy chain | Nanobody B3 | WO2009121948 | 4405 |
| | | variable | | SEQ ID NO: 3 | |
| | | (nanobody) | | | |
| AD183 | BACE4 | Heavy chain | Nanobody B5 | WO2009121948 | 4406 |
| | | variable | | SEQ ID NO: 4 | |
| | | (nanobody) | | | |
| AD184 | BACE5 | Heavy chain | Nanobody B8 | WO2009121948 | 4407 |
| | | variable | | SEQ ID NO: 5 | |
| | | (nanobody) | | | |
| AD185 | BACE6 | Heavy chain | Nanobody B9 | WO2009121948 | 4408 |
| | | variable | | SEQ ID NO: 6 | |
| | | (nanobody) | | | |
| AD186 | BACE7 | Heavy chain | Nanobody B10 | WO2009121948 | 4409 |
| | | variable | | SEQ ID NO: 7 | |
| | | (nanobody) | | | |
| AD187 | BACE8 | Heavy chain | Nanobody B11 | WO2009121948 | 4410 |
| | | variable | | SEQ ID NO: 8 | |
| | | (nanobody) | | | |
| AD188 | BACE9 | Heavy chain | Nanobody B12 | WO2009121948 | 4411 |
| | | variable | | SEQ ID NO: 9 | |
| | | (nanobody) | | | |
| AD189 | amyloid protein | Heavy chain | IG GAMMA-4 | US20100150906 | 4412 |
| | | constant region | CHAIN C REGION | SEQ ID NO: 17 | |
| | | | modified | | |
| AD190 | tau | Heavy chain | hACl-36-3A8-Ab1 | US20150175682 | 4413 |
| | | constant region | and hACl-36-2B6-Ab1 | SEQ ID NO: 14 | |
| AD191 | ApoE | Heavy chain | 2e8 Fab | Trakhanov, S. et al. | 4414 |
| | | fragment | | “Structure of a | |
| | | | | monoclonal 2E8 | |
| | | | | Fab antibody | |
| | | | | fragment specific | |
| | | | | for the low-density | |
| | | | | lipoprotein- | |
| | | | | receptor binding | |
| | | | | region of | |
| | | | | apolipoprotein E | |
| | | | | refined at 1.9 A”, | |
| | | | | Acta Crystallogr. D | |
| | | | | Biol. Crystallogr. | |
| | | | | 55 (PT 1), 122-128 | |
| | | | | (1999), NCBI | |
| | | | | Accession # | |
| | | | | 12E8 P | |
| AD192 | many-growth | Heavy chain fusion | H19L13, H19L16, | U.S. Pat. No. 8,053,569 | 4415 |
| | factors | protein | H19L18, H19L14, | SEQ ID NO: 25 | |
| | | | H19L15, H19L17, | | |
| | | | H19L6, H19L11 | | |
| AD193 | many-growth | Heavy chain fusion | H20L13, H20L16, | U.S. Pat. No. 8,053,569 | 4416 |
| | factors | protein | H20L18, H20L14, | SEQ ID NO: 28 | |
| | | | H20L15, H20L17, | | |
| | | | H20L6, H20L11 | | |
| AD194 | many-growth | Heavy chain fusion | H22L13, H22L16, | U.S. Pat. No. 8,053,569 | 4417 |
| | factors | protein | H22L18, H22L14, | SEQ ID NO: 34 | |
| | | | H22L15, H22L17, | | |
| | | | H22L6, H22L11 | | |
| AD195 | many-growth | Heavy chain fusion | H5L11, H6L11, | U.S. Pat. No. 8,053,569 | 4418 |
| | factors | protein | H14L11, H15L11, | SEQ ID NO: 24 | |
| | | | H16L11, H17L11, | | |
| | | | H18L11, H19L11, | | |
| | | | H20L11, H21L11, | | |
| | | | H22L11, H23L11, | | |
| | | | H24L11, H25L11, | | |
| | | | H700L11 | | |
| AD196 | NOGO | Heavy chain | 2A10 construct | WO2007003421 | 4419 |
| | | humanized | | SEQ ID NO: 79 | |
| | | construct H1 | | | |
| AD197 | NOGO | Heavy chain | 2A10 construct | WO2007003421 | 4420 |
| | | humanized | | SEQ ID NO: 29 | |
| | | construct H14 | | | |
| AD198 | NOGO | Heavy chain | 2A10 construct | WO2007003421 | 4421 |
| | | humanized | | SEQ ID NO: 30 | |
| | | construct H15 | | | |
| AD199 | NOGO | Heavy chain | 2A10 construct | WO2007003421 | 4422 |
| | | humanized | | SEQ ID NO: 31 | |
| | | construct H16 | | | |
| AD200 | NOGO | Heavy chain | 2A10 construct | WO2007003421 | 4423 |
| | | humanized | | SEQ ID NO: 32 | |
| | | construct H17 | | | |
| AD201 | NOGO | Heavy chain | 2A10 construct | WO2007003421 | 4424 |
| | | humanized | | SEQ ID NO: 33 | |
| | | construct H18 | | | |
| AD202 | NOGO | Heavy chain | 2A10 construct | WO2007003421 | 4425 |
| | | humanized | | SEQ ID NO: 92 | |
| | | construct H19 | | | |
| AD203 | NOGO | Heavy chain | 2A10 construct | WO2007003421 | 4426 |
| | | humanized | | SEQ ID NO: 93 | |
| | | construct H20 | | | |
| AD204 | NOGO | Heavy chain | 2A10 construct | WO2007003421 | 4427 |
| | | humanized | | SEQ ID NO: 94 | |
| | | construct H21 | | | |
| AD205 | NOGO | Heavy chain | 2A10 construct | WO2007003421 | 4428 |
| | | humanized | | SEQ ID NO: 95 | |
| | | construct H22 | | | |
| AD206 | NOGO | Heavy chain | 2A10 construct | WO2007003421 | 4429 |
| | | humanized | | SEQ ID NO: 96 | |
| | | construct H23 | | | |
| AD207 | NOGO | Heavy chain | 2A10 construct | WO2007003421 | 4430 |
| | | humanized | | SEQ ID NO: 97 | |
| | | construct H24 | | | |
| AD208 | NOGO | Heavy chain | 2A10 construct | WO2007003421 | 4431 |
| | | humanized | | SEQ ID NO: 98 | |
| | | construct H25 | | | |
| AD209 | NOGO | Heavy chain | 2A10 construct | WO2007003421 | 4432 |
| | | humanized | | SEQ ID NO: 26 | |
| | | construct H5 | | | |
| AD210 | NOGO | Heavy chain | 2A10 construct | WO2007003421 | 4433 |
| | | humanized | | SEQ ID NO: 27 | |
| | | construct H6 | | | |
| AD211 | NOGO | Heavy chain | 2A10 construct | WO2007003421 | 4434 |
| | | humanized | | SEQ ID NO: 28 | |
| | | construct H700 | | | |
| AD212 | RTN4 (NOGO) | Heavy chain IgG4, | Atinumab | U.S. Pat. No. 8,163,285 | 4435 |
| | | immunomodultator | | SEQ ID NO: 24 | |
| AD213 | tau | Heavy chain | ch4E4 | US20150252102 | 4436 |
| | | mature | | SEQ ID NO: 20 | |
| AD214 | tau | Heavy chain | ch4E4(N30Q) | US20150252102 | 4437 |
| | | mature | | SEQ ID NO: 22 | |
| AD215 | A beta | Heavy chain | IR-072 | U.S. Pat. No. 8,858,949 | 4438 |
| | oligomers | variable region | | SEQ ID NO: 1010 | |
| AD216 | A beta | Heavy chain | IR-011 | U.S. Pat. No. 8,858,949 | 4439 |
| | oligomers | variable region | | SEQ ID NO: 114 | |
| AD217 | A beta | Heavy chain | IR-030 | U.S. Pat. No. 8,858,949 | 4440 |
| | oligomers | variable region | | SEQ ID NO: 370 | |
| AD218 | A beta | Heavy chain | IR-031 | U.S. Pat. No. 8,858,949 | 4441 |
| | oligomers | variable region | | SEQ ID NO: 386 | |
| AD219 | A beta | Heavy chain | IR-032 | U.S. Pat. No. 8,858,949 | 4442 |
| | oligomers | variable region | | SEQ ID NO: 402 | |
| AD220 | A beta | Heavy chain | IR-033 | U.S. Pat. No. 8,858,949 | 4443 |
| | oligomers | variable region | | SEQ ID NO: 418 | |
| AD221 | A beta | Heavy chain | IR-034 | U.S. Pat. No. 8,858,949 | 4444 |
| | oligomers | variable region | | SEQ ID NO: 434 | |
| AD222 | A beta | Heavy chain | IR-035 | U.S. Pat. No. 8,858,949 | 4445 |
| | oligomers | variable region | | SEQ ID NO: 450 | |
| AD223 | A beta | Heavy chain | IR-036 | U.S. Pat. No. 8,858,949 | 4446 |
| | oligomers | variable region | | SEQ ID NO: 466 | |
| AD224 | A beta | Heavy chain | IR-037 | U.S. Pat. No. 8,858,949 | 4447 |
| | oligomers | variable region | | SEQ ID NO: 482 | |
| AD225 | A beta | Heavy chain | IR-038 | U.S. Pat. No. 8,858,949 | 4448 |
| | oligomers | variable region | | SEQ ID NO: 498 | |
| AD226 | A beta | Heavy chain | IR-005 | U.S. Pat. No. 8,858,949 | 4449 |
| | oligomers | variable region | | SEQ ID NO: 50 | |
| AD227 | A beta | Heavy chain | IR-081 | U.S. Pat. No. 8,858,949 | 4450 |
| | oligomers | variable region | | SEQ ID NO: 1154 | |
| AD228 | A beta | Heavy chain | IR-039 | U.S. Pat. No. 8,858,949 | 4451 |
| | oligomers | variable region | | SEQ ID NO: 514 | |
| AD229 | A beta | Heavy chain | IR-040 | U.S. Pat. No. 8,858,949 | 4452 |
| | oligomers | variable region | | SEQ ID NO: 530 | |
| AD230 | A beta | Heavy chain | IR-041 | U.S. Pat. No. 8,858,949 | 4453 |
| | oligomers | variable region | | SEQ ID NO: 546 | |
| AD231 | A beta | Heavy chain | IR-043 | U.S. Pat. No. 8,858,949 | 4454 |
| | oligomers | variable region | | SEQ ID NO: 562 | |
| AD232 | A beta | Heavy chain | IR-044 | U.S. Pat. No. 8,858,949 | 4455 |
| | oligomers | variable region | | SEQ ID NO: 578 | |
| AD233 | A beta | Heavy chain | IR-045 | U.S. Pat. No. 8,858,949 | 4456 |
| | oligomers | variable region | | SEQ ID NO: 594 | |
| AD234 | A beta | Heavy chain | IR-046 | U.S. Pat. No. 8,858,949 | 4457 |
| | oligomers | variable region | | SEQ ID NO: 610 | |
| AD235 | A beta | Heavy chain | IR-048 | U.S. Pat. No. 8,858,949 | 4458 |
| | oligomers | variable region | | SEQ ID NO: 626 | |
| AD236 | A beta | Heavy chain | IR-049 | U.S. Pat. No. 8,858,949 | 4459 |
| | oligomers | variable region | | SEQ ID NO: 642 | |
| AD237 | A beta | Heavy chain | IR-050 | U.S. Pat. No. 8,858,949 | 4460 |
| | oligomers | variable region | | SEQ ID NO: 658 | |
| AD238 | A beta | Heavy chain | IR-082 | U.S. Pat. No. 8,858,949 | 4461 |
| | oligomers | variable region | | SEQ ID NO: 1170 | |
| AD239 | A beta | Heavy chain | IR-006 | U.S. Pat. No. 8,858,949 | 4462 |
| | oligomers | variable region | | SEQ ID NO: 66 | |
| AD240 | A beta | Heavy chain | IR-051 | U.S. Pat. No. 8,858,949 | 4463 |
| | oligomers | variable region | | SEQ ID NO: 674 | |
| AD241 | A beta | Heavy chain | IR-052 | U.S. Pat. No. 8,858,949 | 4464 |
| | oligomers | variable region | | SEQ ID NO: 690 | |
| AD242 | A beta | Heavy chain | IR-053 | U.S. Pat. No. 8,858,949 | 4465 |
| | oligomers | variable region | | SEQ ID NO: 706 | |
| AD243 | A beta | Heavy chain | IR-054 | U.S. Pat. No. 8,858,949 | 4466 |
| | oligomers | variable region | | SEQ ID NO: 722 | |
| AD244 | A beta | Heavy chain | IR-055 | U.S. Pat. No. 8,858,949 | 4467 |
| | oligomers | variable region | | SEQ ID NO: 738 | |
| AD245 | A beta | Heavy chain | IR-056 | U.S. Pat. No. 8,858,949 | 4468 |
| | oligomers | variable region | | SEQ ID NO: 754 | |
| AD246 | A beta | Heavy chain | IR-057 | U.S. Pat. No. 8,858,949 | 4469 |
| | oligomers | variable region | | SEQ ID NO: 770 | |
| AD247 | A beta | Heavy chain | IR-058 | U.S. Pat. No. 8,858,949 | 4470 |
| | oligomers | variable region | | SEQ ID NO: 786 | |
| AD248 | A beta | Heavy chain | IR-059 | U.S. Pat. No. 8,858,949 | 4471 |
| | oligomers | variable region | | SEQ ID NO: 802 | |
| AD249 | A beta | Heavy chain | IR-083 | U.S. Pat. No. 8,858,949 | 4472 |
| | oligomers | variable region | | SEQ ID NO: 1186 | |
| AD250 | A beta | Heavy chain | IR-060 | U.S. Pat. No. 8,858,949 | 4473 |
| | oligomers | variable region | | SEQ ID NO: 818 | |
| AD251 | A beta | Heavy chain | IR-007 | U.S. Pat. No. 8,858,949 | 4474 |
| | oligomers | variable region | | SEQ ID NO: 82 | |
| AD252 | A beta | Heavy chain | IR-061 | U.S. Pat. No. 8,858,949 | 4475 |
| | oligomers | variable region | | SEQ ID NO: 834 | |
| AD253 | A beta | Heavy chain | IR-062 | U.S. Pat. No. 8,858,949 | 4476 |
| | oligomers | variable region | | SEQ ID NO: 850 | |
| AD254 | A beta | Heavy chain | IR-063 | U.S. Pat. No. 8,858,949 | 4477 |
| | oligomers | variable region | | SEQ ID NO: 866 | |
| AD255 | A beta | Heavy chain | IR-064 | U.S. Pat. No. 8,858,949 | 4478 |
| | oligomers | variable region | | SEQ ID NO: 882 | |
| AD256 | A beta | Heavy chain | IR-065 | U.S. Pat. No. 8,858,949 | 4479 |
| | oligomers | variable region | | SEQ ID NO: 898 | |
| AD257 | A beta | Heavy chain | IR-066 | U.S. Pat. No. 8,858,949 | 4480 |
| | oligomers | variable region | | SEQ ID NO: 914 | |
| AD258 | A beta | Heavy chain | IR-067 | U.S. Pat. No. 8,858,949 | 4481 |
| | oligomers | variable region | | SEQ ID NO: 930 | |
| AD259 | A beta | Heavy chain | IR-068 | U.S. Pat. No. 8,858,949 | 4482 |
| | oligomers | variable region | | SEQ ID NO: 946 | |
| AD260 | A beta | Heavy chain | IR-084 | U.S. Pat. No. 8,858,949 | 4483 |
| | oligomers | variable region | | SEQ ID NO: 1202 | |
| AD261 | A beta | Heavy chain | IR-069 | U.S. Pat. No. 8,858,949 | 4484 |
| | oligomers | variable region | | SEQ ID NO: 962 | |
| AD262 | A beta | Heavy chain | IR-070 | U.S. Pat. No. 8,858,949 | 4485 |
| | oligomers | variable region | | SEQ ID NO: 978 | |
| AD263 | A beta | Heavy chain | IR-008 | U.S. Pat. No. 8,858,949 | 4486 |
| | oligomers | variable region | | SEQ ID NO: 98 | |
| AD264 | A beta | Heavy chain | IR-071 | U.S. Pat. No. 8,858,949 | 4487 |
| | oligomers | variable region | | SEQ ID NO: 994 | |
| AD265 | A beta | Heavy chain | IR-001 | U.S. Pat. No. 8,858,949 | 4488 |
| | oligomers | variable region | | SEQ ID NO: 2 | |
| AD266 | A beta | Heavy chain | IR-161 | U.S. Pat. No. 8,858,949 | 4489 |
| | oligomers | variable region | | SEQ ID NO: 2878 | |
| AD267 | A beta | Heavy chain | IR-085 | U.S. Pat. No. 8,858,949 | 4490 |
| | oligomers | variable region | | SEQ ID NO: 1218 | |
| AD268 | A beta | Heavy chain | IR-086 | U.S. Pat. No. 8,858,949 | 4491 |
| | oligomers | variable region | | SEQ ID NO: 1234 | |
| AD269 | A beta | Heavy chain | IR-087 | U.S. Pat. No. 8,858,949 | 4492 |
| | oligomers | variable region | | SEQ ID NO: 1250 | |
| AD270 | A beta | Heavy chain | IR-088 | U.S. Pat. No. 8,858,949 | 4493 |
| | oligomers | variable region | | SEQ ID NO: 1266 | |
| AD271 | A beta | Heavy chain | IR-089 | U.S. Pat. No. 8,858,949 | 4494 |
| | oligomers | variable region | | SEQ ID NO: 1282 | |
| AD272 | A beta | Heavy chain | IR-073 | U.S. Pat. No. 8,858,949 | 4495 |
| | oligomers | variable region | | SEQ ID NO: 1026 | |
| AD273 | A beta | Heavy chain | IR-090 | U.S. Pat. No. 8,858,949 | 4496 |
| | oligomers | variable region | | SEQ ID NO: 1298 | |
| AD274 | A beta | Heavy chain | IR-012 | U.S. Pat. No. 8,858,949 | 4497 |
| | oligomers | variable region | | SEQ ID NO: 130 | |
| AD275 | A beta | Heavy chain | IR-092 | U.S. Pat. No. 8,858,949 | 4498 |
| | oligomers | variable region | | SEQ ID NO: 1314 | |
| AD276 | A beta | Heavy chain | IR-093 | U.S. Pat. No. 8,858,949 | 4499 |
| | oligomers | variable region | | SEQ ID NO: 1330 | |
| AD277 | A beta | Heavy chain | IR-094 | U.S. Pat. No. 8,858,949 | 4500 |
| | oligomers | variable region | | SEQ ID NO: 1346 | |
| AD278 | A beta | Heavy chain | IR-095 | U.S. Pat. No. 8,858,949 | 4501 |
| | oligomers | variable region | | SEQ ID NO: 1362 | |
| AD279 | A beta | Heavy chain | IR-097 | U.S. Pat. No. 8,858,949 | 4502 |
| | oligomers | variable region | | SEQ ID NO: 1378 | |
| AD280 | A beta | Heavy chain | IR-098 | U.S. Pat. No. 8,858,949 | 4503 |
| | oligomers | variable region | | SEQ ID NO: 1394 | |
| AD281 | A beta | Heavy chain | IR-100 | U.S. Pat. No. 8,858,949 | 4504 |
| | oligomers | variable region | | SEQ ID NO: 1410 | |
| AD282 | A beta | Heavy chain | IR-101 | U.S. Pat. No. 8,858,949 | 4505 |
| | oligomers | variable region | | SEQ ID NO: 1426 | |
| AD283 | A beta | Heavy chain | IR-074 | U.S. Pat. No. 8,858,949 | 4506 |
| | oligomers | variable region | | SEQ ID NO: 1042 | |
| AD284 | A beta | Heavy chain | IR-102 | U.S. Pat. No. 8,858,949 | 4507 |
| | oligomers | variable region | | SEQ ID NO: 1442 | |
| AD285 | A beta | Heavy chain | IR-104 | U.S. Pat. No. 8,858,949 | 4508 |
| | oligomers | variable region | | SEQ ID NO: 1458 | |
| AD286 | A beta | Heavy chain | IR-013 | U.S. Pat. No. 8,858,949 | 4509 |
| | oligomers | variable region | | SEQ ID NO: 146 | |
| AD287 | A beta | Heavy chain | IR-105 | U.S. Pat. No. 8,858,949 | 4510 |
| | oligomers | variable region | | SEQ ID NO: 1474 | |
| AD288 | A beta | Heavy chain | IR-106 | U.S. Pat. No. 8,858,949 | 4511 |
| | oligomers | variable region | | SEQ ID NO: 1490 | |
| AD289 | A beta | Heavy chain | IR-107 | U.S. Pat. No. 8,858,949 | 4512 |
| | oligomers | variable region | | SEQ ID NO: 1506 | |
| AD290 | A beta | Heavy chain | IR-108 | U.S. Pat. No. 8,858,949 | 4513 |
| | oligomers | variable region | | SEQ ID NO: 1522 | |
| AD291 | A beta | Heavy chain | IR-109 | U.S. Pat. No. 8,858,949 | 4514 |
| | oligomers | variable region | | SEQ ID NO: 1538 | |
| AD292 | A beta | Heavy chain | IR-110 | U.S. Pat. No. 8,858,949 | 4515 |
| | oligomers | variable region | | SEQ ID NO: 1554 | |
| AD293 | A beta | Heavy chain | IR-112 | U.S. Pat. No. 8,858,949 | 4516 |
| | oligomers | variable region | | SEQ ID NO: 1570 | |
| AD294 | A beta | Heavy chain | IR-075 | U.S. Pat. No. 8,858,949 | 4517 |
| | oligomers | variable region | | SEQ ID NO: 1058 | |
| AD295 | A beta | Heavy chain | IR-114 | U.S. Pat. No. 8,858,949 | 4518 |
| | oligomers | variable region | | SEQ ID NO: 1586 | |
| AD296 | A beta | Heavy chain | IR-115 | U.S. Pat. No. 8,858,949 | 4519 |
| | oligomers | variable region | | SEQ ID NO: 1602 | |
| AD297 | A beta | Heavy chain | IR-116 | U.S. Pat. No. 8,858,949 | 4520 |
| | oligomers | variable region | | SEQ ID NO: 1618 | |
| AD298 | A beta | Heavy chain | IR-014 | U.S. Pat. No. 8,858,949 | 4521 |
| | oligomers | variable region | | SEQ ID NO: 162 | |
| AD299 | A beta | Heavy chain | IR-117 | U.S. Pat. No. 8,858,949 | 4522 |
| | oligomers | variable region | | SEQ ID NO: 1634 | |
| AD300 | A beta | Heavy chain | IR-118 | U.S. Pat. No. 8,858,949 | 4523 |
| | oligomers | variable region | | SEQ ID NO: 1650 | |
| AD301 | A beta | Heavy chain | IR-119 | U.S. Pat. No. 8,858,949 | 4524 |
| | oligomers | variable region | | SEQ ID NO: 1666 | |
| AD302 | A beta | Heavy chain | IR-120 | U.S. Pat. No. 8,858,949 | 4525 |
| | oligomers | variable region | | SEQ ID NO: 1682 | |
| AD303 | A beta | Heavy chain | IR-121 | U.S. Pat. No. 8,858,949 | 4526 |
| | oligomers | variable region | | SEQ ID NO: 1698 | |
| AD304 | A beta | Heavy chain | IR-122 | U.S. Pat. No. 8,858,949 | 4527 |
| | oligomers | variable region | | SEQ ID NO: 1714 | |
| AD305 | A beta | Heavy chain | IR-076 | U.S. Pat. No. 8,858,949 | 4528 |
| | oligomers | variable region | | SEQ ID NO: 1074 | |
| AD306 | A beta | Heavy chain | IR-123 | U.S. Pat. No. 8,858,949 | 4529 |
| | oligomers | variable region | | SEQ ID NO: 1730 | |
| AD307 | A beta | Heavy chain | IR-124 | U.S. Pat. No. 8,858,949 | 4530 |
| | oligomers | variable region | | SEQ ID NO: 1746 | |
| AD308 | A beta | Heavy chain | IR-125 | U.S. Pat. No. 8,858,949 | 4531 |
| | oligomers | variable region | | SEQ ID NO: 1762 | |
| AD309 | A beta | Heavy chain | IR-126 | U.S. Pat. No. 8,858,949 | 4532 |
| | oligomers | variable region | | SEQ ID NO: 1778 | |
| AD310 | A beta | Heavy chain | IR-015 | U.S. Pat. No. 8,858,949 | 4533 |
| | oligomers | variable region | | SEQ ID NO: 178 | |
| AD311 | A beta | Heavy chain | IR-127 | U.S. Pat. No. 8,858,949 | 4534 |
| | oligomers | variable region | | SEQ ID NO: 1794 | |
| AD312 | A beta | Heavy chain | IR-002 | U.S. Pat. No. 8,858,949 | 4535 |
| | oligomers | variable region | | SEQ ID NO: 18 | |
| AD313 | A beta | Heavy chain | IR-128 | U.S. Pat. No. 8,858,949 | 4536 |
| | oligomers | variable region | | SEQ ID NO: 1810 | |
| AD314 | A beta | Heavy chain | IR-129 | U.S. Pat. No. 8,858,949 | 4537 |
| | oligomers | variable region | | SEQ ID NO: 1826 | |
| AD315 | A beta | Heavy chain | IR-131 | U.S. Pat. No. 8,858,949 | 4538 |
| | oligomers | variable region | | SEQ ID NO: 1842 | |
| AD316 | A beta | Heavy chain | IR-077 | U.S. Pat. No. 8,858,949 | 4539 |
| | oligomers | variable region | | SEQ ID NO: 1090 | |
| AD317 | A beta | Heavy chain | IR-132 | U.S. Pat. No. 8,858,949 | 4540 |
| | oligomers | variable region | | SEQ ID NO: 1858 | |
| AD318 | A beta | Heavy chain | IR-133 | U.S. Pat. No. 8,858,949 | 4541 |
| | oligomers | variable region | | SEQ ID NO: 1874 | |
| AD319 | A beta | Heavy chain | IR-134 | U.S. Pat. No. 8,858,949 | 4542 |
| | oligomers | variable region | | SEQ ID NO: 1890 | |
| AD320 | A beta | Heavy chain | IR-135 | U.S. Pat. No. 8,858,949 | 4543 |
| | oligomers | variable region | | SEQ ID NO: 1906 | |
| AD321 | A beta | Heavy chain | IR-136 | U.S. Pat. No. 8,858,949 | 4544 |
| | oligomers | variable region | | SEQ ID NO: 1922 | |
| AD322 | A beta | Heavy chain | IR-137 | U.S. Pat. No. 8,858,949 | 4545 |
| | oligomers | variable region | | SEQ ID NO: 1938 | |
| AD323 | A beta | Heavy chain | IR-017 | U.S. Pat. No. 8,858,949 | 4546 |
| | oligomers | variable region | | SEQ ID NO: 194 | |
| AD324 | A beta | Heavy chain | IR-138 | U.S. Pat. No. 8,858,949 | 4547 |
| | oligomers | variable region | | SEQ ID NO: 1954 | |
| AD325 | A beta | Heavy chain | IR-139 | U.S. Pat. No. 8,858,949 | 4548 |
| | oligomers | variable region | | SEQ ID NO: 1970 | |
| AD326 | A beta | Heavy chain | IR-140 | U.S. Pat. No. 8,858,949 | 4549 |
| | oligomers | variable region | | SEQ ID NO: 1986 | |
| AD327 | A beta | Heavy chain | IR-078 | U.S. Pat. No. 8,858,949 | 4550 |
| | oligomers | variable region | | SEQ ID NO: 1106 | |
| AD328 | A beta | Heavy chain | IR-141 | U.S. Pat. No. 8,858,949 | 4551 |
| | oligomers | variable region | | SEQ ID NO: 2002 | |
| AD329 | A beta | Heavy chain | IR-142 | U.S. Pat. No. 8,858,949 | 4552 |
| | oligomers | variable region | | SEQ ID NO: 2018 | |
| AD330 | A beta | Heavy chain | IR-143 | U.S. Pat. No. 8,858,949 | 4553 |
| | oligomers | variable region | | SEQ ID NO: 2034 | |
| AD331 | A beta | Heavy chain | IR-144 | U.S. Pat. No. 8,858,949 | 4554 |
| | oligomers | variable region | | SEQ ID NO: 2050 | |
| AD332 | A beta | Heavy chain | IR-145 | U.S. Pat. No. 8,858,949 | 4555 |
| | oligomers | variable region | | SEQ ID NO: 2066 | |
| AD333 | A beta | Heavy chain | IR-146 | U.S. Pat. No. 8,858,949 | 4556 |
| | oligomers | variable region | | SEQ ID NO: 2082 | |
| AD334 | A beta | Heavy chain | IR-147 | U.S. Pat. No. 8,858,949 | 4557 |
| | oligomers | variable region | | SEQ ID NO: 2098 | |
| AD335 | A beta | Heavy chain | IR-020 | U.S. Pat. No. 8,858,949 | 4558 |
| | oligomers | variable region | | SEQ ID NO: 210 | |
| AD336 | A beta | Heavy chain | IR-149 | U.S. Pat. No. 8,858,949 | 4559 |
| | oligomers | variable region | | SEQ ID NO: 2114 | |
| AD337 | A beta | Heavy chain | IR-150 | U.S. Pat. No. 8,858,949 | 4560 |
| | oligomers | variable region | | SEQ ID NO: 2130 | |
| AD338 | A beta | Heavy chain | IR-079 | U.S. Pat. No. 8,858,949 | 4561 |
| | oligomers | variable region | | SEQ ID NO: 1122 | |
| AD339 | A beta | Heavy chain | IR-151 | U.S. Pat. No. 8,858,949 | 4562 |
| | oligomers | variable region | | SEQ ID NO: 2146 | |
| AD340 | A beta | Heavy chain | IR-152 | U.S. Pat. No. 8,858,949 | 4563 |
| | oligomers | variable region | | SEQ ID NO: 2162 | |
| AD341 | A beta | Heavy chain | IR-153 | U.S. Pat. No. 8,858,949 | 4564 |
| | oligomers | variable region | | SEQ ID NO: 2178 | |
| AD342 | A beta | Heavy chain | IR-154 | U.S. Pat. No. 8,858,949 | 4565 |
| | oligomers | variable region | | SEQ ID NO: 2194 | |
| AD343 | A beta | Heavy chain | IR-155 | U.S. Pat. No. 8,858,949 | 4566 |
| | oligomers | variable region | | SEQ ID NO: 2210 | |
| AD344 | A beta | Heavy chain | IR-156 | U.S. Pat. No. 8,858,949 | 4567 |
| | oligomers | variable region | | SEQ ID NO: 2226 | |
| AD345 | A beta | Heavy chain | IR-157 | U.S. Pat. No. 8,858,949 | 4568 |
| | oligomers | variable region | | SEQ ID NO: 2242 | |
| AD346 | A beta | Heavy chain | IR-158 | U.S. Pat. No. 8,858,949 | 4569 |
| | oligomers | variable region | | SEQ ID NO: 2258 | |
| AD347 | A beta | Heavy chain | IR-021 | U.S. Pat. No. 8,858,949 | 4570 |
| | oligomers | variable region | | SEQ ID NO: 226 | |
| AD348 | A beta | Heavy chain | IR-159 | U.S. Pat. No. 8,858,949 | 4571 |
| | oligomers | variable region | | SEQ ID NO: 2274 | |
| AD349 | A beta | Heavy chain | IR-080 | U.S. Pat. No. 8,858,949 | 4572 |
| | oligomers | variable region | | SEQ ID NO: 1138 | |
| AD350 | A beta | Heavy chain | IR-022 | U.S. Pat. No. 8,858,949 | 4573 |
| | oligomers | variable region | | SEQ ID NO: 242 | |
| AD351 | A beta | Heavy chain | IR-023 | U.S. Pat. No. 8,858,949 | 4574 |
| | oligomers | variable region | | SEQ ID NO: 258 | |
| AD352 | A beta | Heavy chain | IR-024 | U.S. Pat. No. 8,858,949 | 4575 |
| | oligomers | variable region | | SEQ ID NO: 274 | |
| AD353 | A beta | Heavy chain | IR-160 | U.S. Pat. No. 8,858,949 | 4576 |
| | oligomers | variable region | | SEQ ID NO: 2862 | |
| AD354 | A beta | Heavy chain | IR-025 | U.S. Pat. No. 8,858,949 | 4577 |
| | oligomers | variable region | | SEQ ID NO: 290 | |
| AD355 | A beta | Heavy chain | IR-026 | U.S. Pat. No. 8,858,949 | 4578 |
| | oligomers | variable region | | SEQ ID NO: 306 | |
| AD356 | A beta | Heavy chain | IR-027 | U.S. Pat. No. 8,858,949 | 4579 |
| | oligomers | variable region | | SEQ ID NO: 322 | |
| AD357 | A beta | Heavy chain | IR-028 | U.S. Pat. No. 8,858,949 | 4580 |
| | oligomers | variable region | | SEQ ID NO: 338 | |
| AD358 | A beta | Heavy chain | IR-004 | U.S. Pat. No. 8,858,949 | 4581 |
| | oligomers | variable region | | SEQ ID NO: 34 | |
| AD359 | A beta | Heavy chain | IR-029 | U.S. Pat. No. 8,858,949 | 4582 |
| | oligomers | variable region | | SEQ ID NO: 354 | |
| AD360 | AB (1-42) | Heavy chain | 8F5 hum8 VL | US20090232801 | 4583 |
| | Globulomer | variable region | | SEQ ID NO: 1 | |
| AD361 | AB (1-42) | Heavy chain | Hu8F5VHv1 | US20090232801 | 4584 |
| | Globulomer | variable region | | SEQ ID NO: 101 | |
| AD362 | AB (1-42) | Heavy chain | Hu8F5VHv2 | US20090232801 | 4585 |
| | Globulomer | variable region | | SEQ ID NO: 102 | |
| AD363 | AB (1-42) | Heavy chain | Hu8F5VHv1 | US20090232801 | 4586 |
| | Globulomer | variable region | | SEQ ID NO: 108 | |
| AD364 | AB (1-42) | Heavy chain | Hu8F5VHv2 | US20090232801 | 4587 |
| | Globulomer | variable region | | SEQ ID NO: 110 | |
| AD365 | AB (20-42) | Heavy chain | VH 5F7hum8 | US20090175847 | 4588 |
| | Globulomer | variable region | | SEQ ID NO: 1 | |
| AD366 | AB (20-42) | Heavy chain | VH 7C6hum7 | US20090175847 | 4589 |
| | Globulomer | variable region | | SEQ ID NO: 3 | |
| AD367 | ADDL | Heavy chain | | WO2007050359 | 4590 |
| | | variable region | | SEQ ID NO: 108 | |
| AD368 | ADDL | Heavy chain | | WO2007050359 | 4591 |
| | | variable region | | SEQ ID NO: 138 | |
| AD369 | amyloid beta | Heavy chain | | US719576 | 4592 |
| | peptide Aβ | variable region | | SEQ ID NO: 10 | |
| AD370 | amyloid beta | Heavy chain | | US719576 | 4593 |
| | peptide Aβ | variable region | | SEQ ID NO: 8 | |
| AD371 | amyloid | Heavy chain | fl1G3 | U.S. Pat. No. 9,125,846 | 4594 |
| | oligomers | variable region | | SEQ ID NO: 11 | |
| AD372 | amyloid or | Heavy chain | Humanized C2 HIV | WO2008061796 | 4595 |
| | amyloid-like | variable region | AF 4 | SEQ ID NO: 3 | |
| | proteins | | | | |
| AD373 | amyloid protein | Heavy chain | C2 HIV AF 4 | US20100150906 | 4596 |
| | (IGG1 Abeta) | variable region | | SEQ ID NO: 15 | |
| AD374 | amyloid β | Heavy chain | Fv1E1 | U.S. Pat. No. 8,222,002 | 4597 |
| | peptide | variable region | | SEQ ID NO: 1 | |
| AD375 | amyloid β | Heavy chain | VLA2 | U.S. Pat. No. 8,222,002 | 4598 |
| | peptide | variable region | | SEQ ID NO: 101 | |
| AD376 | amyloid β | Heavy chain | Fv1E4 | U.S. Pat. No. 8,222,002 | 4599 |
| | peptide | variable region | | SEQ ID NO: 11 | |
| AD377 | amyloid β | Heavy chain | Fv1E7 | U.S. Pat. No. 8,222,002 | 4600 |
| | peptide | variable region | | SEQ ID NO: 21 | |
| AD378 | amyloid β | Heavy chain | Fv2A7 | U.S. Pat. No. 8,222,002 | 4601 |
| | peptide | variable region | | SEQ ID NO: 31 | |
| AD379 | amyloid β | Heavy chain | Fv2A8 | U.S. Pat. No. 8,222,002 | 4602 |
| | peptide | variable region | | SEQ ID NO: 41 | |
| AD380 | amyloid β | Heavy chain | Fv2B6 | U.S. Pat. No. 8,222,002 | 4603 |
| | peptide | variable region | | SEQ ID NO: 51 | |
| AD381 | amyloid β | Heavy chain | B7 | U.S. Pat. No. 8,222,002 | 4604 |
| | peptide | variable region | | SEQ ID NO: 61 | |
| AD382 | amyloid β | Heavy chain | B6 | U.S. Pat. No. 8,222,002 | 4605 |
| | peptide | variable region | | SEQ ID NO: 71 | |
| AD383 | amyloid β | Heavy chain | F10 | U.S. Pat. No. 8,222,002 | 4606 |
| | peptide | variable region | | SEQ ID NO: 81 | |
| AD384 | amyloid β | Heavy chain | D1 | U.S. Pat. No. 8,222,002 | 4607 |
| | peptide | variable region | | SEQ ID NO: 91 | |
| AD385 | ApoE-CTD | Heavy chain | 807B-M0001-B07 | WO2005051998 | 4608 |
| | | variable region | | SEQ ID NO: 135 | |
| AD386 | ApoE-CTD | Heavy chain | 807B-M0004-A03 | WO2005051998 | 4609 |
| | | variable region | | SEQ ID NO: 136 | |
| AD387 | ApoE-CTD | Heavy chain | 807B-M0004-A05 | WO2005051998 | 4610 |
| | | variable region | | SEQ ID NO: 137 | |
| AD388 | ApoE-CTD | Heavy chain | 807B-M0004-C04 | WO2005051998 | 4611 |
| | | variable region | | SEQ ID NO: 138 | |
| AD389 | ApoE-CTD | Heavy chain | 807B-M0004-C05 | WO2005051998 | 4612 |
| | | variable region | | SEQ ID NO: 139 | |
| AD390 | ApoE-CTD | Heavy chain | 807B-M0004-F06 | WO2005051998 | 4613 |
| | | variable region | | SEQ ID NO: 140 | |
| AD391 | ApoE-CTD | Heavy chain | 807B-M0004-F10 | WO2005051998 | 4614 |
| | | variable region | | SEQ ID NO: 141 | |
| AD392 | ApoE-CTD | Heavy chain | 807B-M0004-H03 | WO2005051998 | 4615 |
| | | variable region | | SEQ ID NO: 142 | |
| AD393 | ApoE-CTD | Heavy chain | 807B-M0009-C03 | WO2005051998 | 4616 |
| | | variable region | | SEQ ID NO: 143 | |
| AD394 | ApoE-CTD | Heavy chain | 807B-M0009-F06 | WO2005051998 | 4617 |
| | | variable region | | SEQ ID NO: 144 | |
| AD395 | ApoE-CTD | Heavy chain | 807B-M0013-A12 | WO2005051998 | 4618 |
| | | variable region | | SEQ ID NO: 145 | |
| AD396 | ApoE-CTD | Heavy chain | 807B-M0079-D10 | WO2005051998 | 4619 |
| | | variable region | | SEQ ID NO: 146 | |
| AD397 | ApoE-CTD | Heavy chain | 807B-M0081-F12 | WO2005051998 | 4620 |
| | | variable region | | SEQ ID NO: 147 | |
| AD398 | ApoE-CTD | Heavy chain | 807B-M0081-H03 | WO2005051998 | 4621 |
| | | variable region | | SEQ ID NO: 148 | |
| AD399 | ApoE-CTD | Heavy chain | 807B-M0083-E11 | WO2005051998 | 4622 |
| | | variable region | | SEQ ID NO: 149 | |
| AD400 | ApoE-CTD | Heavy chain | 807A-M0027-E11 | WO2005051998 | 4623 |
| | | variable region | | SEQ ID NO: 39 | |
| AD401 | ApoE-CTD | Heavy chain | 807A-M0028-B02 | WO2005051998 | 4624 |
| | | variable region | | SEQ ID NO: 40 | |
| AD402 | ApoE-CTD | Heavy chain | 807A-M0026-F05 | WO2005051998 | 4625 |
| | | variable region | | SEQ ID NO: 41 | |
| AD403 | APP | Heavy chain | | WO2014151747 | 4626 |
| | | variable region | | SEQ NO 35 | |
| AD404 | App | Heavy chain | | WO2014151747 | 4627 |
| | | variable region | | SEQ NO 37 | |
| AD405 | APP | Heavy chain | | WO2014151747 | 4628 |
| | | variable region | | SEQ NO 39 | |
| AD406 | APP | Heavy chain | | WO2014151747 | 4629 |
| | | variable region | | SEQ NO 41 | |
| AD407 | APP | Heavy chain | | WO2014151747 | 4630 |
| | | variable region | | SEQ NO 43 | |
| AD408 | Aβ amyloid | Heavy chain | 15C11 | WO2006066049 | 4631 |
| | | variable region | | SEQ ID NO: 4 | |
| AD409 | Aβ amyloid | Heavy chain | 9G8 | WO2006066049 | 4632 |
| | | variable region | | SEQ ID NO: 5 | |
| AD410 | Aβ amyloid | Heavy chain | 266 | WO2006066049 | 4633 |
| | | variable region | | SEQ ID NO: 6 | |
| AD411 | Aβ amyloid | Heavy chain | 12Al 1 vl | WO2006066089 | 4634 |
| | | variable region | | SEQ ID NO: 10 | |
| AD412 | Aβ amyloid | Heavy chain | v2 | WO2006066089 | 4635 |
| | | variable region | | SEQ ID NO: 13 | |
| AD413 | Aβ amyloid | Heavy chain | v2.1 | WO2006066089 | 4636 |
| | | variable region | | SEQ ID NO: 14 | |
| AD414 | Aβ amyloid | Heavy chain | v3 | WO2006066089 | 4637 |
| | | variable region | | SEQ ID NO: 15 | |
| AD415 | Aβ amyloid | Heavy chain | v4.1 | WO2006066089 | 4638 |
| | | variable region | | SEQ ID NO: 16 | |
| AD416 | Aβ amyloid | Heavy chain | v4.2 | WO2006066089 | 4639 |
| | | variable region | | SEQ ID NO: 17 | |
| AD417 | Aβ amyloid | Heavy chain | v4.3 | WO2006066089 | 4640 |
| | | variable region | | SEQ ID NO: 18 | |
| AD418 | Aβ amyloid | Heavy chain | v4.4 | WO2006066089 | 4641 |
| | | variable region | | SEQ ID NO: 19 | |
| AD419 | Aβ amyloid | Heavy chain | v5.1 | WO2006066089 | 4642 |
| | | variable region | | SEQ ID NO: 20 | |
| AD420 | Aβ amyloid | Heavy chain | v5.2 | WO2006066089 | 4643 |
| | | variable region | | SEQ ID NO: 21 | |
| AD421 | Aβ amyloid | Heavy chain | v5.3 | WO2006066089 | 4644 |
| | | variable region | | SEQ ID NO: 22 | |
| AD422 | Aβ amyloid | Heavy chain | v5.4 | WO2006066089 | 4645 |
| | | variable region | | SEQ ID NO: 23 | |
| AD423 | Aβ amyloid | Heavy chain | v5.5 | WO2006066089 | 4646 |
| | | variable region | | SEQ ID NO: 24 | |
| AD424 | Aβ amyloid | Heavy chain | v5.5 | WO2006066089 | 4647 |
| | | variable region | | SEQ ID NO: 25 | |
| AD425 | Aβ amyloid | Heavy chain | v6.1 | WO2006066089 | 4648 |
| | | variable region | | SEQ ID NO: 26 | |
| AD426 | Aβ amyloid | Heavy chain | v6.2 | WO2006066089 | 4649 |
| | | variable region | | SEQ ID NO: 27 | |
| AD427 | Aβ amyloid | Heavy chain | v6.1 | WO2006066089 | 4650 |
| | | variable region | | SEQ ID NO: 28 | |
| AD428 | Aβ amyloid | Heavy chain | v6.2 | WO2006066089 | 4651 |
| | | variable region | | SEQ ID NO: 29 | |
| AD429 | Aβ amyloid | Heavy chain | v7 | WO2006066089 | 4652 |
| | | variable region | | SEQ ID NO: 30 | |
| AD430 | Aβ amyloid | Heavy chain | v8 | WO2006066089 | 4653 |
| | | variable region | | SEQ ID NO: 31 | |
| AD431 | Aβ amyloid | Heavy chain | v3.1 | WO2006066089 | 4654 |
| | | variable region | | SEQ ID NO: 36 | |
| AD432 | Aβ amyloid | Heavy chain | GenBank BAC01733 | WO2006066089 | 4655 |
| | | variable region | | SEQ ID NO: 8 | |
| AD433 | Aβ amyloid | Heavy chain | A19 | WO2006066089 | 4656 |
| | | variable region | | SEQ ID NO: 9 | |
| AD434 | Aβ amyloids | Heavy chain | Humanized 3D6 | U.S. Pat. No. 8,784,810 | 4657 |
| | | variable region | (Bapineuzumab) | SEQ ID NO: 2 | |
| AD435 | Aβ amyloids | Heavy chain | Humanized 10D5 | U.S. Pat. No. 8,784,810 | 4658 |
| | | variable region | | SEQ ID NO: 29 | |
| AD436 | Aβ amyloids | Heavy chain | Humanized 3D6 | U.S. Pat. No. 8,784,810 | 4659 |
| | | variable region | (Bapineuzumab), | SEQ ID NO: 4 | |
| | | | version 2 | | |
| AD437 | Aβ amyloids | Heavy chain | Humanized 12A11 | U.S. Pat. No. 8,784,810 | 4660 |
| | | variable region | | SEQ ID NO: 8 | |
| AD438 | Aβ peptide | Heavy chain | | U.S. Pat. No. 8,066,999 | 4661 |
| | | variable region | | SEQ ID NO: 2 | |
| AD439 | Aβ peptide | Heavy chain | | U.S. Pat. No. 8,066,999 | 4662 |
| | | variable region | | SEQ ID NO: 3 | |
| AD440 | Aβ polypeptide | Heavy chain | preferred embodiment | WO2008084402 | 4663 |
| | | variable region | 6, 11, 12 | SEQ ID NO: 148 | |
| AD441 | Aβ polypeptide | Heavy chain | | WO2008084402 | 4664 |
| | | variable region | | SEQ ID NO: 57 | |
| AD442 | Aβ polypeptide | Heavy chain | | WO2008084402 | 4665 |
| | | variable region | | SEQ ID NO: 58 | |
| AD443 | Aβ polypeptide | Heavy chain | | WO2008084402 | 4666 |
| | | variable region | | SEQ ID NO: 59 | |
| AD444 | Aβ polypeptide | Heavy chain | preferred embodiment | WO2008084402 | 4667 |
| | | variable region | 1, 2, 3, 4, 5, 9 | SEQ ID NO: 60 | |
| AD445 | Aβ polypeptide | Heavy chain | preferred embodiment | WO2008084402 | 4668 |
| | | variable region | 7, 10, 13 | SEQ ID NO: 61 | |
| AD446 | Aβ polypeptide | Heavy chain | preferred embodiment 8 | WO2008084402 | 4669 |
| | | variable region | | SEQ ID NO: 62 | |
| AD447 | Aβ polypeptide | Heavy chain | | WO2008084402 | 4670 |
| | | variable region | | SEQ ID NO: 63 | |
| AD448 | Aβ polypeptide | Heavy chain | | WO2008084402 | 4671 |
| | | variable region | | SEQ ID NO: 64 | |
| AD449 | Aβ polypeptide | Heavy chain | | WO2008084402 | 4672 |
| | | variable region | | SEQ ID NO: 65 | |
| AD450 | Aβ polypeptide | Heavy chain | | WO2008084402 | 4673 |
| | | variable region | | SEQ ID NO: 66 | |
| AD451 | Aβ polypeptide | Heavy chain | | WO2008084402 | 4674 |
| | | variable region | | SEQ ID NO: 67 | |
| AD452 | Aβ polypeptide | Heavy chain | | WO2008084402 | 4675 |
| | | variable region | | SEQ ID NO: 68 | |
| AD453 | Aβ polypeptide | Heavy chain | | WO2008084402 | 4676 |
| | | variable region | | SEQ ID NO: 69 | |
| AD454 | Aβ polypeptide | Heavy chain | | WO2008084402 | 4677 |
| | | variable region | | SEQ ID NO: 70 | |
| AD455 | Aβ polypeptide | Heavy chain | | WO2008084402 | 4678 |
| | | variable region | | SEQ ID NO: 71 | |
| AD456 | beta A4 | Heavy chain | Antibody A | WO2007068429 | 4679 |
| | peptide/Alpha | variable region | | SEQ ID NO: 2 | |
| | beta 4 | | | | |
| AD457 | beta amyloid | Heavy chain | Kabat ID 000333 | U.S. Pat. No. 7,256,273 | 4680 |
| | | variable region | | SEQ ID NO: 34 | |
| AD458 | beta amyloid | Heavy chain | Germline VH4-6 | U.S. Pat. No. 7,256,273 | 4681 |
| | | variable region | | SEQ ID NO: 36 | |
| AD459 | beta amyloid | Heavy chain | Germline VH4-6 | U.S. Pat. No. 7,256,273 | 4682 |
| | | variable region | | SEQ ID NO: 38 | |
| AD460 | beta amyloid | Heavy chain | 12B4 | U.S. Pat. No. 7,256,273 | 4683 |
| | | variable region | | SEQ ID NO: 4 | |
| AD461 | beta amyloid | Heavy chain | humanized 12B4 | U.S. Pat. No. 7,256,273 | 4684 |
| | | variable region | | SEQ ID NO: 8 | |
| AD462 | beta amyloid | Heavy chain | ESBA212 | U.S. Pat. No. 8,323,647 | 4685 |
| | | variable region | | SEQ ID NO: 17 | |
| AD463 | beta amyloid | Heavy chain | Framework 2.3 | U.S. Pat. No. 8,323,647 | 4686 |
| | | variable region | | SEQ ID NO: 18 | |
| AD464 | beta amyloid | Heavy chain | 22C4 | U.S. Pat. No. 8,323,647 | 4687 |
| | | variable region | | SEQ ID NO: 19 | |
| AD465 | beta amyloid | Heavy chain | VH H | U.S. Pat. No. 8,323,647 | 4688 |
| | | variable region | | SEQ ID NO: 20 | |
| AD466 | beta amyloid | Heavy chain | VH I | U.S. Pat. No. 8,323,647 | 4689 |
| | | variable region | | SEQ ID NO: 21 | |
| AD467 | beta amyloid | Heavy chain | VH J | U.S. Pat. No. 8,323,647 | 4690 |
| | | variable region | | SEQ ID NO: 22 | |
| AD468 | beta amyloid | Heavy chain | VH K | U.S. Pat. No. 8,323,647 | 4691 |
| | | variable region | | SEQ ID NO: 23 | |
| AD469 | beta amyloid | Heavy chain | | US10476265 | 4692 |
| | | variable region | | SEQ ID NO: 10 | |
| AD470 | beta amyloid | Heavy chain | | US10476265 | 4693 |
| | | variable region | | SEQ ID NO: 11 | |
| AD471 | beta amyloid | Heavy chain | | US10476265 | 4694 |
| | | variable region | | SEQ ID NO: 12 | |
| AD472 | beta amyloid | Heavy chain | ACI-12-Ab-11 | US20140199323 | 4695 |
| | | variable region | | SEQ ID NO: 10 | |
| AD473 | beta amyloid | Heavy chain | ACI-11-Ab-9 | US20140199323 | 4696 |
| | | variable region | | SEQ ID NO: 8 | |
| AD474 | beta amyloid | Heavy chain | 8C5 | US20150071915 | 4697 |
| | | variable region | | SEQ ID NO: 19 | |
| AD475 | beta amyloid | Heavy chain | 8F5 | US20150071915 | 4698 |
| | | variable region | | SEQ ID NO: 3 | |
| AD476 | beta amyloid | Heavy chain | germline VH3-23 | U.S. Pat. No. 7,189,819 | 4699 |
| | | variable region | | SEQ ID NO: 10 | |
| AD477 | beta amyloid | Heavy chain | | U.S. Pat. No. 7,189,819 | 4700 |
| | | variable region | | SEQ ID NO: 12 | |
| AD478 | beta amyloid | Heavy chain | 10D5 | U.S. Pat. No. 7,189,819 | 4701 |
| | | variable region | | SEQ ID NO: 16 | |
| AD479 | beta amyloid | Heavy chain | m3D6 | U.S. Pat. No. 7,189,819 | 4702 |
| | | variable region | | SEQ ID NO: 4 | |
| AD480 | beta amyloid | Heavy chain | humanized 3D6 | U.S. Pat. No. 7,189,819 | 4703 |
| | | variable region | | SEQ ID NO: 8 | |
| AD481 | beta amyloid | Heavy chain | Kabat ID 109230 | U.S. Pat. No. 7,189,819 | 4704 |
| | | variable region | | SEQ ID NO: 9 | |
| AD482 | beta amyloid | Heavy chain | Bapineuzumab, AAB- | U.S. Pat. No. 8,613,920 | 4705 |
| | | variable region | 001 | SEQ ID NO: 2 | |
| AD483 | beta amyloid | Heavy chain | M99675 | WO2007113172 | 4706 |
| | peptide | variable region | | SEQ ID NO: 21 | |
| AD484 | beta amyloid | Heavy chain | Humanized H1 | WO2007113172 | 4707 |
| | peptide | variable region | | SEQ ID NO: 26 | |
| AD485 | beta amyloid | Heavy chain | Humanized H2 | WO2007113172 | 4708 |
| | peptide | variable region | | SEQ ID NO: 28 | |
| AD486 | beta amyloid | Heavy chain | Humanized H3 | WO2007113172 | 4709 |
| | peptide | variable region | | SEQ ID NO: 30 | |
| AD487 | BETA- | Heavy chain | NI-101.12 | WO2008081008 | 4710 |
| | AMYLOID | variable region | | SEQ ID NO: 10 | |
| AD488 | BETA- | Heavy chain | NI-101.13 | WO2008081008 | 4711 |
| | AMYLOID | variable region | | SEQ ID NO: 14 | |
| AD489 | BETA- | Heavy chain | NI-101.12F6A | WO2008081008 | 4712 |
| | AMYLOID | variable region | | SEQ ID NO: 39 | |
| AD490 | BETA- | Heavy chain | NI-101.10 | WO2008081008 | 4713 |
| | AMYLOID | variable region | | SEQ ID NO: 4 | |
| AD491 | BETA- | Heavy chain | NI-101.13A | WO2008081008 | 4714 |
| | AMYLOID | variable region | | SEQ ID NO: 42 | |
| AD492 | BETA- | Heavy chain | NI-101.13A | WO2008081008 | 4715 |
| | AMYLOID | variable region | | SEQ ID NO: 44 | |
| AD493 | BETA- | Heavy chain | NI-101.11 | WO2008081008 | 4716 |
| | AMYLOID | variable region | | SEQ ID NO: 6 | |
| AD494 | DR6 and P75 | Heavy chain | IP1D6.3 | WO2010062904 | 4717 |
| | | variable region | | SEQ ID NO: 107 | |
| AD495 | DR6 and P75 | Heavy chain | IP2F2.1 | WO2010062904 | 4718 |
| | | variable region | | SEQ ID NO: 117 | |
| AD496 | DR6 and P75 | Heavy chain | IP5D10.2 | WO2010062904 | 4719 |
| | | variable region | | SEQ ID NO: 127 | |
| AD497 | DR6 and P75 | Heavy chain | M51-H09 | WO2010062904 | 4720 |
| | | variable region | | SEQ ID NO: 17 | |
| AD498 | DR6 and P75 | Heavy chain | M53-E04 | WO2010062904 | 4721 |
| | | variable region | | SEQ ID NO: 27 | |
| AD499 | DR6 and P75 | Heavy chain | M53-F04 | WO2010062904 | 4722 |
| | | variable region | | SEQ ID NO: 37 | |
| AD500 | DR6 and P75 | Heavy chain | M62-B02 | WO2010062904 | 4723 |
| | | variable region | | SEQ ID NO: 47 | |
| AD501 | DR6 and P75 | Heavy chain | M63-E10 | WO2010062904 | 4724 |
| | | variable region | | SEQ ID NO: 57 | |
| AD502 | DR6 and P75 | Heavy chain | M66-B03 | WO2010062904 | 4725 |
| | | variable region | | SEQ ID NO: 67 | |
| AD503 | DR6 and P75 | Heavy chain | M50-H01 | WO2010062904 | 4726 |
| | | variable region | | SEQ ID NO: 7 | |
| AD504 | DR6 and P75 | Heavy chain | M67-G02 | WO2010062904 | 4727 |
| | | variable region | | SEQ ID NO: 77 | |
| AD505 | DR6 and P75 | Heavy chain | M77-F03 | WO2010062904 | 4728 |
| | | variable region | | SEQ ID NO: 87 | |
| AD506 | DR6 and P75 | Heavy chain | M73-C04 | WO2010062904 | 4729 |
| | | variable region | | SEQ ID NO: 97 | |
| AD507 | IOD5 | Heavy chain | | WO2002088307 | 4730 |
| | | variable region | | SEQ ID NO: 10 | |
| AD508 | IOD5 | Heavy chain | | WO2002088307 | 4731 |
| | | variable region | | SEQ ID NO: 12 | |
| AD509 | IOD5 | Heavy chain | | WO2002088307 | 4732 |
| | | variable region | | SEQ ID NO: 8 | |
| AD510 | LPG | Heavy chain | #7 | U.S. Pat. No. 8,591,902 | 4733 |
| | (lysophosphatidylglucoside) | variable region | | SEQ ID NO: 18 | |
| AD511 | LPG | Heavy chain | #15 | U.S. Pat. No. 8,591,902 | 4734 |
| | (lysophosphatidylglucoside) | variable region | | SEQ ID NO: 8 | |
| AD512 | MAG | Heavy chain | | U.S. Pat. No. 8,071,731 | 4735 |
| | | variable region | | SEQ ID NO: 13 | |
| AD513 | MAG | Heavy chain | | U.S. Pat. No. 8,071,731 | 4736 |
| | | variable region | | SEQ ID NO: 14 | |
| AD514 | MAG | Heavy chain | | U.S. Pat. No. 8,071,731 | 4737 |
| | | variable region | | SEQ ID NO: 15 | |
| AD515 | MAI (myelin | Heavy chain | | WO2013158748 | 4738 |
| | associated | variable region | | SEQ ID NO: 1 | |
| | inhibitor) | | | | |
| AD516 | MAI (myelin | Heavy chain | | WO2013158748 | 4739 |
| | associated | variable region | | SEQ ID NO: 17 | |
| | inhibitor) | | | | |
| AD517 | NMDA | Heavy chain | | EP2805972 SEQ | 4740 |
| | | variable region | | ID NO: 43 | |
| AD518 | NOGO | Heavy chain | H5L13, H5L16, | US20140147435 | 4741 |
| | | variable region | H5L18, H5L14, | SEQ ID NO: 11 | |
| | | | H5L15, H5L17, H5L6, | | |
| | | | H5L11 | | |
| AD519 | NOGO | Heavy chain | H6L13, H6L16, | US20140147435 | 4742 |
| | | variable region | H6L18, H6L14, | SEQ ID NO: 12 | |
| | | | H6L15, H6L17, H6L6 | | |
| AD520 | NOGO | Heavy chain | H700L13, H700L16, | US20140147435 | 4743 |
| | | variable region | H700L18, H700L14, | SEQ ID NO: 13 | |
| | | | H700L15, H700L17, | | |
| | | | H700L6, H700L11 | | |
| AD521 | NOGO | Heavy chain | H14L13, H14L16, | US20140147435 | 4744 |
| | | variable region | H14L18, H14L14, | SEQ ID NO: 14 | |
| | | | H14L15, H14L17, | | |
| | | | H14L6, H14L11 | | |
| AD522 | NOGO | Heavy chain | H15L13, H15L16, | US20140147435 | 4745 |
| | | variable region | H15L18, H15L14, | SEQ ID NO: 15 | |
| | | | H15L15, H15L17, | | |
| | | | H15L6, H15L11 | | |
| AD523 | NOGO | Heavy chain | H16L13, H16L16, | US20140147435 | 4746 |
| | | variable region | H16L18, H16L14, | SEQ ID NO: 16 | |
| | | | H16L15, H16L17, | | |
| | | | H16L6, H16L11 | | |
| AD524 | NOGO | Heavy chain | H17L13, H17L16, | US20140147435 | 4747 |
| | | variable region | H17L18, H17L14, | SEQ ID NO: 17 | |
| | | | H17L15, H17L17, | | |
| | | | H17L6, H17L11 | | |
| AD525 | NOGO | Heavy chain | H18L13, H18L16, | US20140147435 | 4748 |
| | | variable region | H18L18, H18L14, | SEQ ID NO: 18 | |
| | | | H18L15, H18L17, | | |
| | | | H18L6, H18L11 | | |
| AD526 | NOGO | Heavy chain | H1L13, H1L16, | US20140147435 | 4749 |
| | | variable region | H1L18, H1L14, | SEQ ID NO: 77 | |
| | | | H1L15, H1L17, H1L6 | | |
| AD527 | NOGO | Heavy chain | H19L13, H19L16, | US20140147435 | 4750 |
| | | variable region | H19L18, H19L14, | SEQ ID NO: 85 | |
| | | | H19L15, H19L17, | | |
| | | | H19L6, H19L11 | | |
| AD528 | NOGO | Heavy chain | H20L13, H20L16, | US20140147435 | 4751 |
| | | variable region | H20L18, H20L14, | SEQ ID NO: 86 | |
| | | | H20L15, H20L17, | | |
| | | | H20L6, H20L11 | | |
| AD529 | NOGO | Heavy chain | H21L13, H21L16, | US20140147435 | 4752 |
| | | variable region | H21L18, H21L14, | SEQ ID NO: 87 | |
| | | | H21L15, H21L17, | | |
| | | | H21L6, H21L11 | | |
| AD530 | NOGO | Heavy chain | H22L13, H22L16, | US20140147435 | 4753 |
| | | variable region | H22L18, H22L14, | SEQ ID NO: 88 | |
| | | | H22L15, H22L17, | | |
| | | | H22L6, H22L11 | | |
| AD531 | NOGO | Heavy chain | H23L13, H23L16, | US20140147435 | 4754 |
| | | variable region | H23L18, H23L14, | SEQ ID NO: 89 | |
| | | | H23L15, H23L17, | | |
| | | | H23L6, H23L11 | | |
| AD532 | NOGO | Heavy chain | H24L13, H24L16, | US20140147435 | 4755 |
| | | variable region | H24L18, H24L14, | SEQ ID NO: 90 | |
| | | | H24L15, H24L17, | | |
| | | | H24L6, H24L11 | | |
| AD533 | NOGO | Heavy chain | H25L13, H25L16, | US20140147435 | 4756 |
| | | variable region | H25L18, H25L14, | SEQ ID NO: 91 | |
| | | | H25L15, H25L17, | | |
| | | | H25L6, H25L11 | | |
| AD534 | Nogo-66 | Heavy chain | Antibody clone 50 | US20140065155 | 4757 |
| | | variable region | | SEQ ID NO: 3 | |
| AD535 | Nogo-66 | Heavy chain | Antibody clone 51 | US20140065155 | 4758 |
| | | variable region | | SEQ ID NO: 5 | |
| AD536 | NogoA/NiG | Heavy chain | 6A3-Ig4 | WO2009056509 | 4759 |
| | | variable region | | SEQ ID NO: 24 | |
| AD537 | NogoA/NiG | Heavy chain | 6A3-IgG1 | WO2009056509 | 4760 |
| | | variable region | | SEQ ID NO: 4 | |
| AD538 | N-terminal | Heavy chain | Antibody Tea 1.1 | US20110059092 | 4761 |
| | region of Aβ8- | variable region | (Secreted by | SEQ ID NO: 10 | |
| | x peptide | | Hybridoma IGH525) | | |
| AD539 | N-terminal | Heavy chain | Antibody TeiA 1.6 | US20110059092 | 4762 |
| | region of Aβ8- | variable region | (Secreted by | SEQ ID NO: 2 | |
| | x peptide | | Hybridoma IGH521) | | |
| AD540 | N-terminal | Heavy chain | Antibody TeiA 1.7 | US20110059092 | 4763 |
| | region of Aβ8- | variable region | (Secreted by | SEQ ID NO: 4 | |
| | x peptide | | Hybridoma IGH522) | | |
| AD541 | N-terminal | Heavy chain | Antibody TeiA 1.8 | US20110059092 | 4764 |
| | region of Aβ8- | variable region | (Secreted by | SEQ ID NO: 6 | |
| | x peptide | | Hybridoma IGH523) | | |
| AD542 | N-terminal | Heavy chain | Antibody TeiA 2b.6 | US20110059092 | 4765 |
| | region of Aβ8- | variable region | (Secreted by | SEQ ID NO: 8 | |
| | x peptide | | Hybridoma IGH524) | | |
| AD543 | oligomers of N- | Heavy chain | 9D5 | U.S. Pat. No. 8,795,664 | 4766 |
| | terminal | variable region | | SEQ ID NO: 26 | |
| | truncated Aβ | | | | |
| AD544 | oligomers of N- | Heavy chain | 8C4 | U.S. Pat. No. 8,795,664 | 4767 |
| | terminal | variable region | | SEQ ID NO: 30 | |
| | truncated Aβ | | | | |
| AD545 | PrP | Heavy chain | ICSM18VH | US20140294844 | 4768 |
| | | variable region | | SEQ ID NO: 4 | |
| AD546 | PrPC and/or | Heavy chain | | US20150166668 | 4769 |
| | PrPSc | variable region | | SEQ ID NO: 8 | |
| AD547 | pyroglutamated | Heavy chain | | WO2012136552 | 4770 |
| | A β | variable region | | SEQ ID NO: 25 | |
| AD548 | pyroglutamated | Heavy chain | | WO2012136552 | 4771 |
| | A β | variable region | | SEQ ID NO: 29 | |
| AD549 | pyroglutamated | Heavy chain | | WO2012136552 | 4772 |
| | A β | variable region | | SEQ ID NO: 5 | |
| AD550 | pyroglutamated | Heavy chain | | WO2012136552 | 4773 |
| | A β | variable region | | SEQ ID NO: 9 | |
| AD551 | RGM A | Heavy chain | 5F9.1-GL | US20150183871 | 4774 |
| | | variable region | | SEQ ID NO: 35 | |
| AD552 | RGM A | Heavy chain | 5F9.2-GL | US20150183871 | 4775 |
| | | variable region | | SEQ ID NO: 36 | |
| AD553 | RGM A | Heavy chain | 5F9.3-GL | US20150183871 | 4776 |
| | | variable region | | SEQ ID NO: 37 | |
| AD554 | RGM A | Heavy chain | 5F9.4-GL | US20150183871 | 4777 |
| | | variable region | | SEQ ID NO: 38 | |
| AD555 | RGM A | Heavy chain | 5F9.5-GL | US20150183871 | 4778 |
| | | variable region | | SEQ ID NO: 39 | |
| AD556 | RGM A | Heavy chain | 5F9.6-GL | US20150183871 | 4779 |
| | | variable region | | SEQ ID NO: 40 | |
| AD557 | RGM A | Heavy chain | 5F9.7-GL | US20150183871 | 4780 |
| | | variable region | | SEQ ID NO: 41 | |
| AD558 | RGM A | Heavy chain | 5F9.8-GL | US20150183871 | 4781 |
| | | variable region | | SEQ ID NO: 42 | |
| AD559 | RGM A | Heavy chain | 5F9.9-GL | US20150183871 | 4782 |
| | | variable region | | SEQ ID NO: 43 | |
| AD560 | RGM A | Heavy chain | h5F9.1, h5F9.1, | US20150183871 | 4783 |
| | | variable region | h5F9.1, h5F9.1, | SEQ ID NO: 47 | |
| | | | h5F9.1, h5F9.2, | | |
| | | | h5F9.3 | | |
| AD561 | RGM A | Heavy chain | h5F9.3, h5F9.9, | US20150183871 | 4784 |
| | | variable region | h5F9.25 | SEQ ID NO: 53 | |
| AD562 | RGM A | Heavy chain | h5F9.4, h5F9.10, | US20150183871 | 4785 |
| | | variable region | h5F9.26 | SEQ ID NO: 54 | |
| AD563 | RGMa | Heavy chain | AE12-1 | US20140023659 | 4786 |
| | | variable region | | SEQ ID NO: 1 | |
| AD564 | RGMa | Heavy chain | AE12-20 | US20140023659 | 4787 |
| | | variable region | | SEQ ID NO: 107 | |
| AD565 | RGMa | Heavy chain | AE12-21 | US20140023659 | 4788 |
| | | variable region | | SEQ ID NO: 115 | |
| AD566 | RGMa | Heavy chain | AE12-23 | US20140023659 | 4789 |
| | | variable region | | SEQ ID NO: 123 | |
| AD567 | RGMa | Heavy chain | AE12-24 | US20140023659 | 4790 |
| | | variable region | | SEQ ID NO: 131 | |
| AD568 | RGMa | Heavy chain | AE12-3 | US20140023659 | 4791 |
| | | variable region | | SEQ ID NO: 17 | |
| AD569 | RGMa | Heavy chain | AE12-4 | US20140023659 | 4792 |
| | | variable region | | SEQ ID NO: 25 | |
| AD570 | RGMa | Heavy chain | AE12-5 | US20140023659 | 4793 |
| | | variable region | | SEQ ID NO: 33 | |
| AD571 | RGMa | Heavy chain | AE12-6 | US20140023659 | 4794 |
| | | variable region | | SEQ ID NO: 41 | |
| AD572 | RGMa | Heavy chain | AE12-7 | US20140023659 | 4795 |
| | | variable region | | SEQ ID NO: 49 | |
| AD573 | RGMa | Heavy chain | AE12-8 | US20140023659 | 4796 |
| | | variable region | | SEQ ID NO: 57 | |
| AD574 | RGMa | Heavy chain | AE12-2 | US20140023659 | 4797 |
| | | variable region | | SEQ ID NO: 9 | |
| AD575 | RGMa | Heavy chain | AE12-13 | US20140023659 | 4798 |
| | | variable region | | SEQ ID NO: 91 | |
| AD576 | RGMa | Heavy chain | AE12-15 | US20140023659 | 4799 |
| | | variable region | | SEQ ID NO: 99 | |
| AD577 | tau | Heavy chain | | WO2014100600 | 4800 |
| | | variable region | | SEQ ID NO: 45 | |
| AD578 | tau | Heavy chain | NI-105.24B2 | US20150252102 | 4801 |
| | | variable region | | SEQ ID NO: 13 | |
| AD579 | tau | Heavy chain | NI-105.4A3 | US20150252102 | 4802 |
| | | variable region | | SEQ ID NO: 17 | |
| AD580 | tau | Heavy chain | NI-105.4E4 | US20150252102 | 4803 |
| | | variable region | | SEQ ID NO: 9 | |
| AD581 | tau | Heavy chain | | WO2013041962 | 4804 |
| | | variable region | | SEQ ID NO: 138 | |
| AD582 | tau | Heavy chain | | WO2013041962 | 4805 |
| | | variable region | | SEQ ID NO: 139 | |
| AD583 | tau | Heavy chain | | WO2013041962 | 4806 |
| | | variable region | | SEQ ID NO: 140 | |
| AD584 | tau | Heavy chain | | WO2013041962 | 4807 |
| | | variable region | | SEQ ID NO: 145 | |
| AD585 | tau | Heavy chain | | WO2013041962 | 4808 |
| | | variable region | | SEQ ID NO: 147 | |
| AD586 | tau | Heavy chain | | WO2013041962 | 4809 |
| | | variable region | | SEQ ID NO: 148 | |
| AD587 | tau | Heavy chain | | WO2014100600 | 4810 |
| | | variable region | | SEQ ID NO: 220 | |
| AD588 | tau | Heavy chain | NI-105.17C1 | WO2014100600 | 4811 |
| | | variable region | | SEQ ID NO: 44 | |
| AD589 | tau | Heavy chain | | WO2014100600 | 4812 |
| | | variable region | | SEQ ID NO: 47 | |
| AD590 | tau | Heavy chain | NI-105.6C5 | WO2014100600 | 4813 |
| | | variable region | | SEQ ID NO: 48 | |
| AD591 | tau | Heavy chain | NI-105.29G10 | WO2014100600 | 4814 |
| | | variable region | | SEQ ID NO: 50 | |
| AD592 | tau | Heavy chain | NI-105.6L9 | WO2014100600 | 4815 |
| | | variable region | | SEQ ID NO: 52 | |
| AD593 | tau | Heavy chain | NI-105.40E8 | WO2014100600 | 4816 |
| | | variable region | | SEQ ID NO: 54 | |
| AD594 | tau | Heavy chain | NI-105.48E5 | WO2014100600 | 4817 |
| | | variable region | | SEQ ID NO: 56 | |
| AD595 | tau | Heavy chain | NI-105.6E3 | WO2014100600 | 4818 |
| | | variable region | | SEQ ID NO: 58 | |
| AD596 | tau | Heavy chain | NI-105.22E1 | WO2014100600 | 4819 |
| | | variable region | | SEQ ID NO: 60 | |
| AD597 | tau | Heavy chain | NI-105.26B12 | WO2014100600 | 4820 |
| | | variable region | | SEQ ID NO: 62 | |
| AD598 | tau | Heavy chain | NI-105.12E12 | WO2014100600 | 4821 |
| | | variable region | | SEQ ID NO: 65 | |
| AD599 | tau | Heavy chain | NI-105.60E7 | WO2014100600 | 4822 |
| | | variable region | | SEQ ID NO: 67 | |
| AD600 | tau | Heavy chain | NI-105.14E2 | WO2014100600 | 4823 |
| | | variable region | | SEQ ID NO: 69 | |
| AD601 | tau | Heavy chain | NI-105.39E2 | WO2014100600 | 4824 |
| | | variable region | | SEQ ID NO: 71 | |
| AD602 | tau | Heavy chain | NI-105.19C6 | WO2014100600 | 4825 |
| | | variable region | | SEQ ID NO: 73 | |
| AD603 | tau | Heavy chain | | WO2014100600 | 4826 |
| | | variable region | | SEQ ID NO: 75 | |
| AD604 | tau | Heavy chain | NI-105.9C4 | WO2014100600 | 4827 |
| | | variable region | | SEQ ID NO: 76 | |
| AD605 | tau | Heavy chain | IPN002 variant 1 | U.S. Pat. No. 8,926,974 | 4828 |
| | | variable region | | SEQ ID NO: 36 | |
| AD606 | tau | Heavy chain | IPN002 variant 2 | U.S. Pat. No. 8,926,974 | 4829 |
| | | variable region | | SEQ ID NO: 37 | |
| AD607 | tau | Heavy chain | IPN002 variant 3 | U.S. Pat. No. 8,926,974 | 4830 |
| | | variable region | | SEQ ID NO: 38 | |
| AD608 | tau | Heavy chain | IPN002 variant 4 | U.S. Pat. No. 8,926,974 | 4831 |
| | | variable region | | SEQ ID NO: 39 | |
| AD609 | tau | Heavy chain | PT1 | US20150307600 | 4832 |
| | | variable region | | SEQ ID NO: 35 | |
| AD610 | tau | Heavy chain | PT3 | US20150307600 | 4833 |
| | | variable region | | SEQ ID NO: 37 | |
| AD611 | tau | Heavy chain | | U.S. Pat. No. 9,304,138 | 4834 |
| | | variable region | | SEQ ID NO: 1 | |
| AD612 | tau | Heavy chain | | U.S. Pat. No. 9,304,138 | 4835 |
| | | variable region | | SEQ ID NO: 2 | |
| AD613 | tau | Heavy chain | | U.S. Pat. No. 9,304,138 | 4836 |
| | | variable region | | SEQ ID NO: 3 | |
| AD614 | tau | Heavy chain | | U.S. Pat. No. 9,304,138 | 4837 |
| | | variable region | | SEQ ID NO: 4 | |
| AD615 | tau | Heavy chain | | U.S. Pat. No. 9,304,138 | 4838 |
| | | variable region | | SEQ ID NO: 5 | |
| AD616 | tau | Heavy chain | | U.S. Pat. No. 9,304,138 | 4839 |
| | | variable region | | SEQ ID NO: 68 | |
| AD617 | tau | Heavy chain | | U.S. Pat. No. 9,304,138 | 4840 |
| | | variable region | | SEQ ID NO: 76 | |
| AD618 | tau | Heavy chain | | U.S. Pat. No. 9,304,138 | 4841 |
| | | variable region | | SEQ ID NO: 88 | |
| AD619 | tau | Heavy chain | | U.S. Pat. No. 9,304,138 | 4842 |
| | | variable region | | SEQ ID NO: 96 | |
| AD620 | tau | Heavy chain | | U.S. Pat. No. 9,304,138 | 4843 |
| | | variable region | | SEQ ID NO: 104 | |
| AD621 | tau | Heavy chain | hACl-36-3A8-Ab1 | US20150175682 | 4844 |
| | | variable region | and hACl-36-2B6-Ab1 | SEQ ID NO: 7 | |
| AD622 | tau | Heavy chain | hACl-36-3A8-Ab1.v2. | US20150175682 | 4845 |
| | | variable region | | SEQ ID NO: 20 | |
| AD623 | tau | Heavy chain | hACl-36-2B6-Ab1.v2 | US20150175682 | 4846 |
| | | variable region | | SEQ ID NO: 21 | |
| AD624 | tau | Heavy chain | ADx210 | US20140161875 | 4847 |
| | | variable region | | SEQ ID NO: 15 | |
| AD625 | tau | Heavy chain | ADx210 subpart | US20140161875 | 4848 |
| | | variable region | | SEQ ID NO: 17 | |
| AD626 | tau | Heavy chain | ADx215 | US20140161875 | 4849 |
| | | variable region | | SEQ ID NO: 25 | |
| AD627 | tau antigen | Heavy chain | ADx202 | WO2015004163 | 4850 |
| | | variable region | | SEQ ID NO: 14 | |
| AD628 | tau ps 422 | Heavy chain | antibody Mab2.10.3 | US20110059093 | 4851 |
| | | variable region | | SEQ ID NO: 2 | |
| AD629 | tau ps 422 | Heavy chain | Mab 005 | US20110059093 | 4852 |
| | | variable region | | SEQ ID NO: 22 | |
| AD630 | tau ps 422 | Heavy chain | Mab 019 | US20110059093 | 4853 |
| | | variable region | | SEQ ID NO: 30 | |
| AD631 | tau ps 422 | Heavy chain | Mab 020 | US20110059093 | 4854 |
| | | variable region | | SEQ ID NO: 38 | |
| AD632 | tau ps 422 | Heavy chain | Mab 085 | US20110059093 | 4855 |
| | | variable region | | SEQ ID NO: 46 | |
| AD633 | tau ps 422 | Heavy chain | Mab 086 | US20110059093 | 4856 |
| | | variable region | | SEQ ID NO: 54 | |
| AD634 | tau ps 422 | Heavy chain | Mab 097 | US20110059093 | 4857 |
| | | variable region | | SEQ ID NO: 62 | |
| AD635 | TrkA | Heavy chain | HuVHWO | WO2009098238 | 4858 |
| | | variable region | | SEQ ID NO: 17 | |
| AD636 | NOGO | Heavy chain | 2A10 construct | WO2007003421 | 4859 |
| | | variable region | | SEQ ID NO: 77 | |
| | | humanized | | | |
| | | construct H1 | | | |
| AD637 | NOGO | Heavy chain | 2A10 construct | WO2007003421 | 4860 |
| | | variable region | | SEQ ID NO: 14 | |
| | | humanized | | | |
| | | construct H14 | | | |
| AD638 | NOGO | Heavy chain | 2A10 construct | WO2007003421 | 4861 |
| | | variable region | | SEQ ID NO: 15 | |
| | | humanized | | | |
| | | construct H15 | | | |
| AD639 | NOGO | Heavy chain | 2A10 construct | WO2007003421 | 4862 |
| | | variable region | | SEQ ID NO: 16 | |
| | | humanized | | | |
| | | construct H16 | | | |
| AD640 | NOGO | Heavy chain | 2A10 construct | WO2007003421 | 4863 |
| | | variable region | | SEQ ID NO: 17 | |
| | | humanized | | | |
| | | construct H17 | | | |
| AD641 | NOGO | Heavy chain | 2A10 construct | WO2007003421 | 4864 |
| | | variable region | | SEQ ID NO: 18 | |
| | | humanized | | | |
| | | construct H18 | | | |
| AD642 | NOGO | Heavy chain | 2A10 construct | WO2007003421 | 4865 |
| | | variable region | | SEQ ID NO: 85 | |
| | | humanized | | | |
| | | construct H19 | | | |
| AD643 | NOGO | Heavy chain | 2A10 construct | WO2007003421 | 4866 |
| | | variable region | | SEQ ID NO: 86 | |
| | | humanized | | | |
| | | construct H20 | | | |
| AD644 | NOGO | Heavy chain | 2A10 construct | WO2007003421 | 4867 |
| | | variable region | | SEQ ID NO: 87 | |
| | | humanized | | | |
| | | construct H21 | | | |
| AD645 | NOGO | Heavy chain | 2A10 construct | WO2007003421 | 4868 |
| | | variable region | | SEQ ID NO: 88 | |
| | | humanized | | | |
| | | construct H22 | | | |
| AD646 | NOGO | Heavy chain | 2A10 construct | WO2007003421 | 4869 |
| | | variable region | | SEQ ID NO: 89 | |
| | | humanized | | | |
| | | construct H23 | | | |
| AD647 | NOGO | Heavy chain | 2A10 construct | WO2007003421 | 4870 |
| | | variable region | | SEQ ID NO: 90 | |
| | | humanized | | | |
| | | construct H24 | | | |
| AD648 | NOGO | Heavy chain | 2A10 construct | WO2007003421 | 4871 |
| | | variable region | | SEQ ID NO: 91 | |
| | | humanized | | | |
| | | construct H25 | | | |
| AD649 | NOGO | Heavy chain | 2A10 construct | WO2007003421 | 4872 |
| | | variable region | | SEQ ID NO: 11 | |
| | | humanized | | | |
| | | construct H5 | | | |
| AD650 | NOGO | Heavy chain | 2A10 construct | WO2007003421 | 4873 |
| | | variable region | | SEQ ID NO: 12 | |
| | | humanized | | | |
| | | construct H6 | | | |
| AD651 | NOGO | Heavy chain | 2A10 construct | WO2007003421 | 4874 |
| | | variable region | | SEQ ID NO: 13 | |
| | | humanized | | | |
| | | construct H700 | | | |
| AD652 | beta A4 | Heavy chain with | Antibody A | WO2007068429 | 4875 |
| | peptide/Alpha | Fc region | | SEQ ID NO: 26 | |
| | beta 8 | | | | |
| AD653 | ACTH | Light chain | Ab3 | WO2015127288 | 4876 |
| | | | | SEQ ID NO: 101 | |
| AD654 | ACTH | Light chain | Ab4 | WO2015127288 | 4877 |
| | | | | SEQ ID NO: 141 | |
| AD655 | ACTH | Light chain | Ab5 | WO2015127288 | 4878 |
| | | | | SEQ ID NO: 181 | |
| AD656 | ACTH | Light chain | Ab1 | WO2015127288 | 4879 |
| | | | | SEQ ID NO: 21 | |
| AD657 | ACTH | Light chain | Ab6 | WO2015127288 | 4880 |
| | | | | SEQ ID NO: 221 | |
| AD658 | ACTH | Light chain | Ab7 | WO2015127288 | 4881 |
| | | | | SEQ ID NO: 261 | |
| AD659 | ACTH | Light chain | Ab9 | WO2015127288 | 4882 |
| | | | | SEQ ID NO: 301 | |
| AD660 | ACTH | Light chain | Ab10 | WO2015127288 | 4883 |
| | | | | SEQ ID NO: 341 | |
| AD661 | ACTH | Light chain | Ab11 | WO2015127288 | 4884 |
| | | | | SEQ ID NO: 381 | |
| AD662 | ACTH | Light chain | Ab12 | WO2015127288 | 4885 |
| | | | | SEQ ID NO: 421 | |
| AD663 | ACTH | Light chain | Ab1.H | WO2015127288 | 4886 |
| | | | | SEQ ID NO: 461 | |
| AD664 | ACTH | Light chain | Ab2.H | WO2015127288 | 4887 |
| | | | | SEQ ID NO: 501 | |
| AD665 | ACTH | Light chain | Ab3.H | WO2015127288 | 4888 |
| | | | | SEQ ID NO: 541 | |
| AD666 | ACTH | Light chain | Ab4.H | WO2015127288 | 4889 |
| | | | | SEQ ID NO: 581 | |
| AD667 | ACTH | Light chain | Ab2 | WO2015127288 | 4890 |
| | | | | SEQ ID NO: 61 | |
| AD668 | ACTH | Light chain | Ab6.H | WO2015127288 | 4891 |
| | | | | SEQ ID NO: 621 | |
| AD669 | ACTH | Light chain | Ab7.H | WO2015127288 | 4892 |
| | | | | SEQ ID NO: 661 | |
| AD670 | ACTH | Light chain | Ab7A.H | WO2015127288 | 4893 |
| | | | | SEQ ID NO: 701 | |
| AD671 | ACTH | Light chain | Ab10.H | WO2015127288 | 4894 |
| | | | | SEQ ID NO: 741 | |
| AD672 | ACTH | Light chain | Ab11.H | WO2015127288 | 4895 |
| | | | | SEQ ID NO: 781 | |
| AD673 | ACTH | Light chain | Ab11A.H | WO2015127288 | 4896 |
| | | | | SEQ ID NO: 821 | |
| AD674 | ACTH | Light chain | Ab12.H | WO20151.27288 | 4897 |
| | | | | SEQ ID NO: 861 | |
| AD675 | Alpha beta | Light chain | Gantenerumab | Immunogenetics | 4898 |
| | fibril | | | Informition | |
| | | | | System; CHAIN | |
| | | | | ID NO: 8894_L. | |
| AD676 | amyloid beta | Light chain | | US719,576 | 4899 |
| | peptide Aβ | | | SEQ ID NO: 11 | |
| AD677 | Amyloid | Light chain | 3 Fab of Yw412.8.31 | Wang, W. et al. “A | 4900 |
| | beta/BACE1 | | | Therapeutic | |
| | | | | Antibody Targeting | |
| | | | | BACE1 Inhibits | |
| | | | | Amyloid NO: - | |
| | | | | {beta}Production | |
| | | | | in Vivo” Sci Transl | |
| | | | | Med 3 (84), | |
| | | | | 84RA43 (2011), | |
| | | | | NCBI Accession # | |
| | | | | 3RIG_L (222aa) | |
| AD678 | amyloid or | Light chain | Humanized C2 | WO2008061796 | 4901 |
| | amyloid-like | | | SEQ ID NO: 2 | |
| | proteins | | | | |
| AD679 | amyloid protein | Light chain | C2 | US20100150906 | 4902 |
| | | | | SEQ ID NO: 13 | |
| AD680 | amyloids | Light chain | #118 | WO2010012004 | 4903 |
| | | | | SEQ ID NO: 10 | |
| AD681 | amyloids | Light chain | #121 | WO2010012004 | 4904 |
| | | | | SEQ ID NO: 12 | |
| AD682 | amyloids | Light chain | #201 | WO2010012004 | 4905 |
| | | | | SEQ ID NO: 14 | |
| AD683 | amyloids | Light chain | #204 | WO2010012004 | 4906 |
| | | | | SEQ ID NO: 15 | |
| AD684 | amyloids | Light chain | #205 | WO2010012004 | 4907 |
| | | | | SEQ ID NO: 17 | |
| AD685 | APP | Light chain | F5.100 | WO2014151747 | 4908 |
| | | | | SEQ ID NO: | |
| AD686 | APP | Light chain | BBS1 MAb | WO2014151747 | 4909 |
| | | | | SEQ ID NO: 25 | |
| AD687 | APP | Light chain | F5.87 | WO2014151747 | 4910 |
| | | | | SEQ ID NO: 27 | |
| AD688 | APP | Light chain | F5.87 | WO2014151747 | 4911 |
| | | | | SEQ ID NO: 54 | |
| AD689 | Aβ amyloids | Light chain | Humanized 12A11, | U.S. Pat. No. 8,784,810 | 4912 |
| | | | version 2 | SEQ ID NO: 10 | |
| AD690 | Aβ amyloids | Light chain | Humanized 3D6 | U.S. Pat. No. 8,784,810 | 4913 |
| | | | (Bapineuzumab), | SEQ ID NO: 6 | |
| | | | version 3 | | |
| AD691 | beta A4 | Light chain | Antibody A | WO2007068429 | 4914 |
| | peptide/Alpha | | | SEQ ID NO: 8 | |
| | beta 6 | | | | |
| AD692 | beta A4 | Light chain | Antibody A | WO2007068429 | 4915 |
| | peptide/Alpha | | | SEQ ID NO: 22 | |
| | beta 7 | | | | |
| AD693 | beta amyloid | Light chain | | U.S. Pat. No. 10,476,265 | 4916 |
| | | | | SEQ ID NO: 19 | |
| AD694 | beta amyloid | Light chain | | U.S. Pat. No. 13,319,710 | 4917 |
| | | | | SEQ ID NO: 22 | |
| AD695 | beta amyloid | Light chain | | U.S. Pat. No. 13,319,710 | 4918 |
| | | | | SEQ ID NO: 28 | |
| AD696 | beta amyloid | Light chain | (13C3) | U.S. Pat. No. 13,319,710 | 4919 |
| | | | | SEQ ID NO: 4 | |
| AD697 | beta amyloid | Light chain | C2 | US20070166311 | 4920 |
| | | | | SEQ ID NO: 21 | |
| AD698 | beta amyloid | Light chain | Solanezumab | Immunogenetics | 4921 |
| | peptide | | | Information | |
| | | | | System; CHAIN | |
| | | | | ID NO: 9097_L. | |
| AD699 | beta amyloid | Light chain | Mature L1 | WO2007113172 | 4922 |
| | peptide | | | SEQ ID NO: 40 | |
| AD700 | beta-amyloid | Light chain | Aducanumab, | | 4923 |
| | | | BIIB0307 | | |
| AD701 | EAG1 | Light chain | chimeric ImAb3 | WO2006037604 | 4924 |
| | | | | SEQ ID NO: 10 | |
| AD702 | EAG1 | Light chain | chimeric ImAb4 | WO2006037604 | 4925 |
| | | | | SEQ ID NO: 14 | |
| AD703 | EAG1 | Light chain | LC-lmAb3-humB3 | WO2006037604 | 4926 |
| | | | | SEQ ID NO: 18 | |
| AD704 | EAG1 | Light chain | ImAb4 | WO2006037604 | 4927 |
| | | | | SEQ ID NO: 2 | |
| AD705 | EAG1 | Light chain | LC-lmAb4-humA17 | WO2006037604 | 4928 |
| | | | | SEQ ID NO: 22 | |
| AD706 | EAG1 | Light chain | LC-lmAb3-humA3 | WO2006037604 | 4929 |
| | | | | SEQ ID NO: 26 | |
| AD707 | EAG1 | Light chain | LC-lmAb3-humA17 | WO2006037604 | 4930 |
| | | | | SEQ ID NO: 30 | |
| AD708 | EAG1 | Light chain | LC-lmAb4-humA5-1 | WO2006037604 | 4931 |
| | | | | SEQ ID NO: 34 | |
| AD709 | EAG1 | Light chain | LC-lmAb4-humO1 | WO2006037604 | 4932 |
| | | | | SEQ ID NO: 38 | |
| AD710 | EAG1 | Light chain | ImAb3 | WO2006037604 | 4933 |
| | | | | SEQ ID NO: 6 | |
| AD711 | IGG1 Abeta | Light chain | Humanized C2 | US20090155249 | 4934 |
| | | | | SEQ ID NO: 13 | |
| AD712 | NOGO | Light chain | H6L13 FL, H19L13 | US20140147435 | 4935 |
| | | | FL, H20L13 FL, | SEQ ID NO: 35 | |
| | | | H21L13 FL, H25L13 | | |
| | | | FL | | |
| AD713 | NOGO | Light chain | H16L16 FL, H19L16 | US20140147435 | 4936 |
| | | | FL, H20L16 FL, | SEQ ID NO: 38 | |
| | | | H21L16 FL, H25L16 | | |
| | | | FL, H18L16 FL | | |
| AD714 | NOGO | Light chain | H16L18 FL, H19L18 | US20140147435 | 4937 |
| | | | FL, H20L18 FL, | SEQ ID NO: 40 | |
| | | | H21L18 FL, H25L18 | | |
| | | | FL | | |
| AD715 | Nogo receptor-1 | Light chain | 7E11 | US20090215691 | 4938 |
| | | | | SEQ ID NO: 15 | |
| AD716 | Nogo receptor-2 | Light chain | 7E11 | US20090215691 | 4939 |
| | | | | SEQ ID NO: 17 | |
| AD717 | PrPC and/or | Light chain | | US20150166668 | 4940 |
| | PrPSc | | | SEQ ID NO: 9 | |
| AD718 | PrPC and/or | Light chain | | U.S. Pat. No. 8,852,587 | 4941 |
| | PrPSc | | | SEQ ID NO: 5 | |
| AD719 | tau | Light chain | hACl-36-3A8 Ab1, | WO2013151762 | 4942 |
| | | | hACl-36-3A8 Ab1.v2, | SEQ ID NO: 22 | |
| | | | hACl-36-3A8 Ab1.v3, | | |
| | | | hACl-36-3A8 Ab1.v4 | | |
| AD720 | tau | Light chain | hACl-36-3B8 Ab1, | WO2013151762 | 4943 |
| | | | hACl-36-3B8 Ab1.v2, | SEQ ID NO: 23 | |
| | | | hACl-36-3B8 Ab1.v3, | | |
| | | | hACl-36-3B8 Ab1.v4 | | |
| AD721 | tau | Light chain | IPN001 | U.S. Pat. No. 8,980,271 | 4944 |
| | | | | SEQ ID NO: 13 | |
| AD722 | tau | Light chain | IPN002 | U.S. Pat. No. 8,980,271 | 4945 |
| | | | | SEQ ID NO: 15 | |
| AD723 | tau | Light chain | hACl-36-3A8- | US20150175682 | 4946 |
| | | | Ab1 and hACl-36- | SEQ ID NO: 18 | |
| | | | 2B6-Ab1 | | |
| AD724 | tau | Light chain | hACl-36-3A8-Ab1 | US20150175682 | 4947 |
| | | | (IgG4), hACl-36-3A8- | SEQ ID NO: 22 | |
| | | | Ab1.v2 (IgG4), hACl- | | |
| | | | 36-3A8-Ab1.v3 | | |
| | | | (IgG1), and hACl-36- | | |
| | | | 3A8-Ab1.v4 (IgG1 | | |
| | | | N297G) | | |
| AD725 | tau | Light chain | hACl-36-2B6-Ab1 | US20150175682 | 4948 |
| | | | (IgG4), hACl-36-2B6- | SEQ ID NO: 23 | |
| | | | Ab1.v2 (IgG4), hACl- | | |
| | | | 36-2B6-Ab1.v3 | | |
| | | | (IgG1), and hACl-36- | | |
| | | | 2B6-Ab1.v4 (IgG1 | | |
| | | | N297G) | | |
| AD726 | tau | Light chain | hACl-36-3A8-Ab1 | US20150175682 | 4949 |
| | | | (IgG4) | SEQ ID NO: 24 | |
| AD727 | TrkA | Light chain | BXhVL4 | WO2009098238 | 4950 |
| | | | | SEQ ID NO: 10 | |
| AD728 | TrkA | Light chain | BXhVL5 | WO2009098238 | 4951 |
| | | | | SEQ ID NO: 11 | |
| AD729 | TrkA | Light chain | BXhVLβ | WO2009098238 | 4952 |
| | | | | SEQ ID NO: 12 | |
| AD730 | TrkA | Light chain | BXhVL7 | WO2009098238 | 4953 |
| | | | | SEQ ID NO: 13 | |
| AD731 | TrkA | Light chain | BXhVL8 | WO2009098238 | 4954 |
| | | | | SEQ ID NO: 14 | |
| AD732 | TrkA | Light chain | mVLEP | WO2009098238 | 4955 |
| | | | | SEQ ID NO: 16 | |
| AD733 | TrkA | Light chain | BXhVL1 | WO2009098238 | 4956 |
| | | | | SEQ ID NO: 7 | |
| AD734 | TrkA | Light chain | BXhVL2 | WO2009098238 | 4957 |
| | | | | SEQ ID NO: 8 | |
| AD735 | TrkA | Light chain | BXhVL3 | WO2009098238 | 4958 |
| | | | | SEQ ID NO: 9 | |
| AD736 | trk-C (NT-3 | Light chain | 2250 | U.S. Pat. No. 7,615,383 | 4959 |
| | trkC ligand) | | | SEQ ID NO: 49 | |
| AD737 | trk-C (NT-3 | Light chain | 2253 | U.S. Pat. No. 7,615,383 | 4960 |
| | trkC ligand) | | | SEQ ID NO: 50 | |
| AD738 | trk-C (NT-3 | Light chain | 2256 | U.S. Pat. No. 7,615,383 | 4961 |
| | trkC ligand) | | | SEQ ID NO: 51 | |
| AD739 | trk-C (NT-3 | Light chain | 6.1.2 | U.S. Pat. No. 7,615,383 | 4962 |
| | trkC ligand) | | | SEQ ID NO: 52 | |
| AD740 | trk-C (NT-3 | Light chain | 6.4.1 | U.S. Pat. No. 7,615,383 | 4963 |
| | trkC ligand) | | | SEQ ID NO: 53 | |
| AD741 | trk-C (NT-3 | Light chain | 2345 | U.S. Pat. No. 7,615,383 | 4964 |
| | trkC ligand) | | | SEQ ID NO: 54 | |
| AD742 | trk-C (NT-3 | Light chain | 2349 | U.S. Pat. No. 7,615,383 | 4965 |
| | trkC ligand) | | | SEQ ID NO: 55 | |
| AD743 | | Light chain | Crenezumab light | | 4966 |
| | | | CHAIN | | |
| AD744 | | Light chain | Gantenerumab light | | 4967 |
| | | | chain | | |
| AD745 | | Light chain | Ponezumab light | | 4968 |
| | | | CHAIN | | |
| AD746 | | Light chain | Solanezumab light | | 4969 |
| | | | CHAIN | | |
| AD747 | amyloid protein | Light chain | C2 | US20100150906 | 4970 |
| | | constant region | | SEQ ID NO: 14 | |
| AD748 | IGG1 Abeta | Light Chain | Humanized C2 | US20090155249 | 4971 |
| | | constant region | | SEQ ID NO: 14 | |
| AD749 | ApoE | Light chain | 2e8 Fab | Trakhanov, S. et al. | 4972 |
| | | fragment | | “Structure of a | |
| | | | | monoclonal 2E8 | |
| | | | | Fab antibody | |
| | | | | fragment specific | |
| | | | | for the low-density | |
| | | | | lipoprotein- | |
| | | | | receptor binding | |
| | | | | region of | |
| | | | | apolipoprotein E | |
| | | | | refined at 1.9 A”, | |
| | | | | Acta Crystallogr. D | |
| | | | | Biol. Crystallogr. | |
| | | | | 55 (PT 1), 122-128 | |
| | | | | (1999), NCBI | |
| | | | | Accession # | |
| | | | | 12E8 M | |
| AD750 | many - growth | Light chain fusion | H21L13, H21L16, | U.S. Pat. No. 8,053,569 | 4973 |
| | factors | protein | H21L18, H21L14, | SEQ ID NO: 31 | |
| | | | H21L15, H21L17, | | |
| | | | H21L6, H21L11 | | |
| AD751 | many - growth | Light chain fusion | H23L13, H23L16, | U.S. Pat. No. 8,053,569 | 4974 |
| | factors | protein | H23L18, H23L14, | SEQ ID NO: 36 | |
| | | | H23L15, H23L17, | | |
| | | | H23L6, H23L11 | | |
| AD752 | NOGO | Light chain | 2A10 construct | WO2007003421 | 4975 |
| | | humanized | | SEQ ID NO: 80 | |
| | | construct L11 | | | |
| AD753 | NOGO | Light chain | 2A10 construct | WO2007003421 | 4976 |
| | | humanized | | SEQ ID NO: 35 | |
| | | construct L13 | | | |
| AD754 | NOGO | Light chain | 2A10 construct | WO2007003421 | 4977 |
| | | humanized | | SEQ ID NO: 36 | |
| | | construct L14 | | | |
| AD755 | NOGO | Light chain | 2A10 construct | WO2007003421 | 4978 |
| | | humanized | | SEQ ID NO: 37 | |
| | | construct L15 | | | |
| AD756 | NOGO | Light chain | 2A10 construct | WO2007003421 | 4979 |
| | | humanized | | SEQ ID NO: 38 | |
| | | construct L16 | | | |
| AD757 | NOGO | Light chain | 2A10 construct | WO2007003421 | 4980 |
| | | humanized | | SEQ ID NO: 39 | |
| | | construct L17 | | | |
| AD758 | NOGO | Light chain | 2A10 construct | WO2007003421 | 4981 |
| | | humanized | | SEQ ID NO: 40 | |
| | | construct L18 | | | |
| AD759 | NOGO | Light chain | 2A10 construct | WO2007003421 | 4982 |
| | | humanized | | SEQ ID NO: 34 | |
| | | construct L6 | | | |
| AD760 | RTN4 | Light chain IgG4, | Atinumab | U.S. Pat. No. 8,163,285 | 4983 |
| | | immunomodulator | | SEQ ID NO: 25 | |
| AD761 | tau | Light chain mature | ch4E4 | US20150252102 | 4984 |
| | | | | SEQ ID NO: 21 | |
| AD762 | A beta | Light chain | IR-008 | U.S. Pat. No. 8,858,949 | 4985 |
| | oligomers | variable region | | SEQ ID NO: 100 | |
| AD763 | A beta | Light chain | IR-072 | U.S. Pat. No. 8,858,949 | 4986 |
| | oligomers | variable region | | SEQ ID NO: 1012 | |
| AD764 | A beta | Light chain | IR-073 | U.S. Pat. No. 8,858,949 | 4987 |
| | oligomers | variable region | | SEQ ID NO: 1028 | |
| AD765 | A beta | Light chain | IR-074 | U.S. Pat. No. 8,858,949 | 4988 |
| | oligomers | variable region | | SEQ ID NO: 1044 | |
| AD766 | A beta | Light chain | IR-075 | U.S. Pat. No. 8,858,949 | 4989 |
| | oligomers | variable region | | SEQ ID NO: 1060 | |
| AD767 | A beta | Light chain | IR-076 | U.S. Pat. No. 8,858,949 | 4990 |
| | oligomers | variable region | | SEQ ID NO: 1076 | |
| AD768 | A beta | Light chain | IR-077 | U.S. Pat. No. 8,858,949 | 4991 |
| | oligomers | variable region | | SEQ ID NO: 1092 | |
| AD769 | A beta | Light chain | IR-078 | U.S. Pat. No. 8,858,949 | 4992 |
| | oligomers | variable region | | SEQ ID NO: 1108 | |
| AD770 | A beta | Light chain | IR-079 | U.S. Pat. No. 8,858,949 | 4993 |
| | oligomers | variable region | | SEQ ID NO: 1124 | |
| AD771 | A beta | Light chain | IR-080 | U.S. Pat. No. 8,858,949 | 4994 |
| | oligomers | variable region | | SEQ ID NO: 1140 | |
| AD772 | A beta | Light chain | IR-081 | U.S. Pat. No. 8,858,949 | 4995 |
| | oligomers | variable region | | SEQ ID NO: 1156 | |
| AD773 | A beta | Light chain | IR-011 | U.S. Pat. No. 8,858,949 | 4996 |
| | oligomers | variable region | | SEQ ID NO: 116 | |
| AD774 | A beta | Light chain | IR-082 | U.S. Pat. No. 8,858,949 | 4997 |
| | oligomers | variable region | | SEQ ID NO: 1172 | |
| AD775 | A beta | Light chain | IR-083 | U.S. Pat. No. 8,858,949 | 4998 |
| | oligomers | variable region | | SEQ ID NO: 1188 | |
| AD776 | A beta | Light chain | IR-084 | U.S. Pat. No. 8,858,949 | 4999 |
| | oligomers | variable region | | SEQ ID NO: 1204 | |
| AD777 | A beta | Light chain | IR-085 | U.S. Pat. No. 8,858,949 | 5000 |
| | oligomers | variable region | | SEQ ID NO: 1220 | |
| AD778 | A beta | Light chain | IR-086 | U.S. Pat. No. 8,858,949 | 5001 |
| | oligomers | variable region | | SEQ ID NO: 1236 | |
| AD779 | A beta | Light chain | IR-087 | U.S. Pat. No. 8,858,949 | 5002 |
| | oligomers | variable region | | SEQ ID NO: 1252 | |
| AD780 | A beta | Light chain | IR-088 | U.S. Pat. No. 8,858,949 | 5003 |
| | oligomers | variable region | | SEQ ID NO: 1268 | |
| AD781 | A beta | Light chain | IR-089 | U.S. Pat. No. 8,858,949 | 5004 |
| | oligomers | variable region | | SEQ ID NO: 1284 | |
| AD782 | A beta | Light chain | IR-090 | U.S. Pat. No. 8,858,949 | 5005 |
| | oligomers | variable region | | SEQ ID NO: 1300 | |
| AD783 | A beta | Light chain | IR-092 | U.S. Pat. No. 8,858,949 | 5006 |
| | oligomers | variable region | | SEQ ID NO: 1316 | |
| AD784 | A beta | Light chain | IR-012 | U.S. Pat. No. 8,858,949 | 5007 |
| | oligomers | variable region | | SEQ ID NO: 132 | |
| AD785 | A beta | Light chain | IR-093 | U.S. Pat. No. 8,858,949 | 5008 |
| | oligomers | variable region | | SEQ ID NO: 1332 | |
| AD786 | A beta | Light chain | IR-094 | U.S. Pat. No. 8,858,949 | 5009 |
| | oligomers | variable region | | SEQ ID NO: 1348 | |
| AD787 | A beta | Light chain | IR-095 | U.S. Pat. No. 8,858,949 | 5010 |
| | oligomers | variable region | | SEQ ID NO: 1364 | |
| AD788 | A beta | Light chain | IR-097 | U.S. Pat. No. 8,858,949 | 5011 |
| | oligomers | variable region | | SEQ ID NO: 1380 | |
| AD789 | A beta | Light chain | IR-098 | U.S. Pat. No. 8,858,949 | 5012 |
| | oligomers | variable region | | SEQ ID NO: 1396 | |
| AD790 | A beta | Light chain | IR-100 | U.S. Pat. No. 8,858,949 | 5013 |
| | oligomers | variable region | | SEQ ID NO: 1412 | |
| AD791 | A beta | Light chain | IR-101 | U.S. Pat. No. 8,858,949 | 5014 |
| | oligomers | variable region | | SEQ ID NO: 1428 | |
| AD792 | A beta | Light chain | IR-102 | U.S. Pat. No. 8,858,949 | 5015 |
| | oligomers | variable region | | SEQ ID NO: 1444 | |
| AD793 | A beta | Light chain | IR-104 | U.S. Pat. No. 8,858,949 | 5016 |
| | oligomers | variable region | | SEQ ID NO: 1460 | |
| AD794 | A beta | Light chain | IR-105 | U.S. Pat. No. 8,858,949 | 5017 |
| | oligomers | variable region | | SEQ ID NO: 1476 | |
| AD795 | A beta | Light chain | IR-013 | U.S. Pat. No. 8,858,949 | 5018 |
| | oligomers | variable region | | SEQ ID NO: 148 | |
| AD796 | A beta | Light chain | IR-106 | U.S. Pat. No. 8,858,949 | 5019 |
| | oligomers | variable region | | SEQ ID NO: 1492 | |
| AD797 | A beta | Light chain | IR-107 | U.S. Pat. No. 8,858,949 | 5020 |
| | oligomers | variable region | | SEQ ID NO: 1508 | |
| AD798 | A beta | Light chain | IR-108 | U.S. Pat. No. 8,858,949 | 5021 |
| | oligomers | variable region | | SEQ ID NO: 1524 | |
| AD799 | A beta | Light chain | IR-109 | U.S. Pat. No. 8,858,949 | 5022 |
| | oligomers | variable region | | SEQ ID NO: 1540 | |
| AD800 | A beta | Light chain | IR-110 | U.S. Pat. No. 8,858,949 | 5023 |
| | oligomers | variable region | | SEQ ID NO: 1556 | |
| AD801 | A beta | Light chain | IR-112 | U.S. Pat. No. 8,858,949 | 5024 |
| | oligomers | variable region | | SEQ ID NO: 1572 | |
| AD802 | A beta | Light chain | IR-114 | U.S. Pat. No. 8,858,949 | 5025 |
| | oligomers | variable region | | SEQ ID NO: 1588 | |
| AD803 | A beta | Light chain | IR-115 | U.S. Pat. No. 8,858,949 | 5026 |
| | oligomers | variable region | | SEQ ID NO: 1604 | |
| AD804 | A beta | Light chain | IR-116 | U.S. Pat. No. 8,858,949 | 5027 |
| | oligomers | variable region | | SEQ ID NO: 1620 | |
| AD805 | A beta | Light chain | IR-117 | U.S. Pat. No. 8,858,949 | 5028 |
| | oligomers | variable region | | SEQ ID NO: 1636 | |
| AD806 | A beta | Light chain | IR-014 | U.S. Pat. No. 8,858,949 | 5029 |
| | oligomers | variable region | | SEQ ID NO: 164 | |
| AD807 | A beta | Light chain | IR-118 | U.S. Pat. No. 8,858,949 | 5030 |
| | oligomers | variable region | | SEQ ID NO: 1652 | |
| AD808 | A beta | Light chain | IR-119 | U.S. Pat. No. 8,858,949 | 5031 |
| | oligomers | variable region | | SEQ ID NO: 1668 | |
| AD809 | A beta | Light chain | IR-120 | U.S. Pat. No. 8,858,949 | 5032 |
| | oligomers | variable region | | SEQ ID NO: 1684 | |
| AD810 | A beta | Light chain | IR-121 | U.S. Pat. No. 8,858,949 | 5033 |
| | oligomers | variable region | | SEQ ID NO: 1700 | |
| AD811 | A beta | Light chain | IR-122 | U.S. Pat. No. 8,858,949 | 5034 |
| | oligomers | variable region | | SEQ ID NO: 1716 | |
| AD812 | A beta | Light chain | IR-123 | U.S. Pat. No. 8,858,949 | 5035 |
| | oligomers | variable region | | SEQ ID NO: 1732 | |
| AD813 | A beta | Light chain | IR-124 | U.S. Pat. No. 8,858,949 | 5036 |
| | oligomers | variable region | | SEQ ID NO: 1748 | |
| AD814 | A beta | Light chain | IR-125 | U.S. Pat. No. 8,858,949 | 5037 |
| | oligomers | variable region | | SEQ ID NO: 1764 | |
| AD815 | A beta | Light chain | IR-126 | U.S. Pat. No. 8,858,949 | 5038 |
| | oligomers | variable region | | SEQ ID NO: 1780 | |
| AD816 | A beta | Light chain | IR-127 | U.S. Pat. No. 8,858,949 | 5039 |
| | oligomers | variable region | | SEQ ID NO: 1796 | |
| AD817 | A beta | Light chain | IR-015 | U.S. Pat. No. 8,858,949 | 5040 |
| | oligomers | variable region | | SEQ ID NO: 180 | |
| AD818 | A beta | Light chain | IR-128 | U.S. Pat. No. 8,858,949 | 5041 |
| | oligomers | variable region | | SEQ ID NO: 1812 | |
| AD819 | A beta | Light chain | IR-129 | U.S. Pat. No. 8,858,949 | 5042 |
| | oligomers | variable region | | SEQ ID NO: 1828 | |
| AD820 | A beta | Light chain | IR-131 | U.S. Pat. No. 8,858,949 | 5043 |
| | oligomers | variable region | | SEQ ID NO: 1844 | |
| AD821 | A beta | Light chain | IR-132 | U.S. Pat. No. 8,858,949 | 5044 |
| | oligomers | variable region | | SEQ ID NO: 1860 | |
| AD822 | A beta | Light chain | IR-133 | U.S. Pat. No. 8,858,949 | 5045 |
| | oligomers | variable region | | SEQ ID NO: 1876 | |
| AD823 | A beta | Light chain | IR-134 | U.S. Pat. No. 8,858,949 | 5046 |
| | oligomers | variable region | | SEQ ID NO: 1892 | |
| AD824 | A beta | Light chain | IR-135 | U.S. Pat. No. 8,858,949 | 5047 |
| | oligomers | variable region | | SEQ ID NO: 1908 | |
| AD825 | A beta | Light chain | IR-136 | U.S. Pat. No. 8,858,949 | 5048 |
| | oligomers | variable region | | SEQ ID NO: 1924 | |
| AD826 | A beta | Light chain | IR-137 | U.S. Pat. No. 8,858,949 | 5049 |
| | oligomers | variable region | | SEQ ID NO: 1940 | |
| AD827 | A beta | Light chain | IR-138 | U.S. Pat. No. 8,858,949 | 5050 |
| | oligomers | variable region | | SEQ ID NO: 1956 | |
| AD828 | A beta | Light chain | IR-017 | U.S. Pat. No. 8,858,949 | 5051 |
| | oligomers | variable region | | SEQ ID NO: 196 | |
| AD829 | A beta | Light chain | IR-139 | U.S. Pat. No. 8,858,949 | 5052 |
| | oligomers | variable region | | SEQ ID NO: 1972 | |
| AD830 | A beta | Light chain | IR-140 | U.S. Pat. No. 8,858,949 | 5053 |
| | oligomers | variable region | | SEQ ID NO: 1988 | |
| AD831 | A beta | Light chain | IR-002 | U.S. Pat. No. 8,858,949 | 5054 |
| | oligomers | variable region | | SEQ ID NO: 20 | |
| AD832 | A beta | Light chain | IR-141 | U.S. Pat. No. 8,858,949 | 5055 |
| | oligomers | variable region | | SEQ ID NO: 2004 | |
| AD833 | A beta | Light chain | IR-142 | U.S. Pat. No. 8,858,949 | 5056 |
| | oligomers | variable region | | SEQ ID NO: 2020 | |
| AD834 | A beta | Light chain | IR-143 | U.S. Pat. No. 8,858,949 | 5057 |
| | oligomers | variable region | | SEQ ID NO: 2036 | |
| AD835 | A beta | Light chain | IR-144 | U.S. Pat. No. 8,858,949 | 5058 |
| | oligomers | variable region | | SEQ ID NO: 2052 | |
| AD836 | A beta | Light chain | IR-145 | U.S. Pat. No. 8,858,949 | 5059 |
| | oligomers | variable region | | SEQ ID NO: 2068 | |
| AD837 | A beta | Light chain | IR-146 | U.S. Pat. No. 8,858,949 | 5060 |
| | oligomers | variable region | | SEQ ID NO: 2084 | |
| AD838 | A beta | Light chain | IR-147 | U.S. Pat. No. 8,858,949 | 5061 |
| | oligomers | variable region | | SEQ ID NO: 2100 | |
| AD839 | A beta | Light chain | IR-149 | U.S. Pat. No. 8,858,949 | 5062 |
| | oligomers | variable region | | SEQ ID NO: 2116 | |
| AD840 | A beta | Light chain | IR-020 | U.S. Pat. No. 8,858,949 | 5063 |
| | oligomers | variable region | | SEQ ID NO: 212 | |
| AD841 | A beta | Light chain | IR-150 | U.S. Pat. No. 8,858,949 | 5064 |
| | oligomers | variable region | | SEQ ID NO: 2132 | |
| AD842 | A beta | Light chain | IR-151 | U.S. Pat. No. 8,858,949 | 5065 |
| | oligomers | variable region | | SEQ ID NO: 2148 | |
| AD843 | A beta | Light chain | IR-152 | U.S. Pat. No. 8,858,949 | 5066 |
| | oligomers | variable region | | SEQ ID NO: 2164 | |
| AD844 | A beta | Light chain | IR-153 | U.S. Pat. No. 8,858,949 | 5067 |
| | oligomers | variable region | | SEQ ID NO: 2180 | |
| AD845 | A beta | Light chain | IR-154 | U.S. Pat. No. 8,858,949 | 5068 |
| | oligomers | variable region | | SEQ ID NO: 2196 | |
| AD846 | A beta | Light chain | IR-155 | U.S. Pat. No. 8,858,949 | 5069 |
| | oligomers | variable region | | SEQ ID NO: 2212 | |
| AD847 | A beta | Light chain | IR-156 | U.S. Pat. No. 8,858,949 | 5070 |
| | oligomers | variable region | | SEQ ID NO: 2228 | |
| AD848 | A beta | Light chain | IR-157 | U.S. Pat. No. 8,858,949 | 5071 |
| | oligomers | variable region | | SEQ ID NO: 2244 | |
| AD849 | A beta | Light chain | IR-158 | U.S. Pat. No. 8,858,949 | 5072 |
| | oligomers | variable region | | SEQ ID NO: 2260 | |
| AD850 | A beta | Light chain | IR-159 | U.S. Pat. No. 8,858,949 | 5073 |
| | oligomers | variable region | | SEQ ID NO: 2276 | |
| AD851 | A beta | Light chain | IR-021 | U.S. Pat. No. 8,858,949 | 5074 |
| | oligomers | variable region | | SEQ ID NO: 228 | |
| AD852 | A beta | Light chain | IR-022 | U.S. Pat. No. 8,858,949 | 5075 |
| | oligomers | variable region | | SEQ ID NO: 244 | |
| AD853 | A beta | Light chain | IR-023 | U.S. Pat. No. 8,858,949 | 5076 |
| | oligomers | variable region | | SEQ ID NO: 260 | |
| AD854 | A beta | Light chain | IR-024 | U.S. Pat. No. 8,858,949 | 5077 |
| | oligomers | variable region | | SEQ ID NO: 276 | |
| AD855 | A beta | Light chain | IR-160 | U.S. Pat. No. 8,858,949 | 5078 |
| | oligomers | variable region | | SEQ ID NO: 2864 | |
| AD856 | A beta | Light chain | IR-161 | U.S. Pat. No. 8,858,949 | 5079 |
| | oligomers | variable region | | SEQ ID NO: 2880 | |
| AD857 | A beta | Light chain | IR-025 | U.S. Pat. No. 8,858,949 | 5080 |
| | oligomers | variable region | | SEQ ID NO: 292 | |
| AD858 | A beta | Light chain | IR-026 | U.S. Pat. No. 8,858,949 | 5081 |
| | oligomers | variable region | | SEQ ID NO: 308 | |
| AD859 | A beta | Light chain | IR-027 | U.S. Pat. No. 8,858,949 | 5082 |
| | oligomers | variable region | | SEQ ID NO: 324 | |
| AD860 | A beta | Light chain | IR-028 | U.S. Pat. No. 8,858,949 | 5083 |
| | oligomers | variable region | | SEQ ID NO: 340 | |
| AD861 | A beta | Light chain | IR-029 | U.S. Pat. No. 8.858,949 | 5084 |
| | oligomers | variable region | | SEQ ID NO: 356 | |
| AD862 | A beta | Light chain | IR-004 | U.S. Pat. No. 8,858,949 | 5085 |
| | oligomers | variable region | | SEQ ID NO: 36 | |
| AD863 | A beta | Light chain | IR-030 | U.S. Pat. No. 8,858,949 | 5086 |
| | oligomers | variable region | | SEQ ID NO: 372 | |
| AD864 | A beta | Light chain | IR-031 | U.S. Pat. No. 8,858,949 | 5087 |
| | oligomers | variable region | | SEQ ID NO: 388 | |
| AD865 | A beta | Light chain | IR-001 | U.S. Pat. No. 8,858,949 | 5088 |
| | oligomers | variable region | | SEQ ID NO: 4 | |
| AD866 | A beta | Light chain | IR-032 | U.S. Pat. No. 8,858,949 | 5089 |
| | oligomers | variable region | | SEQ ID NO: 404 | |
| AD867 | A beta | Light chain | IR-033 | U.S. Pat. No. 8,858,949 | 5090 |
| | oligomers | variable region | | SEQ ID NO: 420 | |
| AD868 | A beta | Light chain | IR-034 | U.S. Pat. No. 8,858,949 | 5091 |
| | oligomers | variable region | | SEQ ID NO: 436 | |
| AD869 | A beta | Light chain | IR-035 | U.S. Pat. No. 8,858,949 | 5092 |
| | oligomers | variable region | | SEQ ID NO: 452 | |
| AD870 | A beta | Light chain | IR-036 | U.S. Pat. No. 8,858,949 | 5093 |
| | oligomers | variable region | | SEQ ID NO: 468 | |
| AD871 | A beta | Light chain | IR-037 | U.S. Pat. No. 8,858,949 | 5094 |
| | oligomers | variable region | | SEQ ID NO: 484 | |
| AD872 | A beta | Light chain | IR-038 | U.S. Pat. No. 8,858,949 | 5095 |
| | oligomers | variable region | | SEQ ID NO: 500 | |
| AD873 | A beta | Light chain | IR-039 | U.S. Pat. No. 8,858,949 | 5096 |
| | oligomers | variable region | | SEQ ID NO: 516 | |
| AD874 | A beta | Light chain | IR-005 | U.S. Pat. No. 8,858,949 | 5097 |
| | oligomers | variable region | | SEQ ID NO: 52 | |
| AD875 | A beta | Light chain | IR-040 | U.S. Pat. No. 8,858,949 | 5098 |
| | oligomers | variable region | | SEQ ID NO: 532 | |
| AD876 | A beta | Light chain | IR-041 | U.S. Pat. No. 8,858,949 | 5099 |
| | oligomers | variable region | | SEQ ID NO: 548 | |
| AD877 | A beta | Light chain | IR-043 | U.S. Pat. No. 8,858,949 | 5100 |
| | oligomers | variable region | | SEQ ID NO: 564 | |
| AD878 | A beta | Light chain | IR-044 | U.S. Pat. No. 8,858,949 | 5101 |
| | oligomers | variable region | | SEQ ID NO: 580 | |
| AD879 | A beta | Light chain | IR-045 | U.S. Pat. No. 8,858,949 | 5102 |
| | oligomers | variable region | | SEQ ID NO: 596 | |
| AD880 | A beta | Light chain | IR-046 | U.S. Pat. No. 8,858,949 | 5103 |
| | oligomers | variable region | | SEQ ID NO: 612 | |
| AD881 | A beta | Light chain | IR-048 | U.S. Pat. No. 8,858,949 | 5104 |
| | oligomers | variable region | | SEQ ID NO: 628 | |
| AD882 | A beta | Light chain | IR-049 | U.S. Pat. No. 8,858,949 | 5105 |
| | oligomers | variable region | | SEQ ID NO: 644 | |
| AD883 | A beta | Light chain | IR-050 | U.S. Pat. No. 8,858,949 | 5106 |
| | oligomers | variable region | | SEQ ID NO: 660 | |
| AD884 | A beta | Light chain | IR-051 | U.S. Pat. No. 8,858,949 | 5107 |
| | oligomers | variable region | | SEQ ID NO: 676 | |
| AD885 | A beta | Light chain | IR-006 | U.S. Pat. No. 8,858,949 | 5108 |
| | oligomers | variable region | | SEQ ID NO: 68 | |
| AD886 | A beta | Light chain | IR-052 | U.S. Pat. No. 8,858,949 | 5109 |
| | oligomers | variable region | | SEQ ID NO: 692 | |
| AD887 | A beta | Light chain | IR-053 | U.S. Pat. No. 8,858,949 | 5110 |
| | oligomers | variable region | | SEQ ID NO: 708 | |
| AD888 | A beta | Light chain | IR-054 | U.S. Pat. No. 8,858,949 | 5111 |
| | oligomers | variable region | | SEQ ID NO: 724 | |
| AD889 | A beta | Light chain | IR-055 | U.S. Pat. No. 8,858,949 | 5112 |
| | oligomers | variable region | | SEQ ID NO: 740 | |
| AD890 | A beta | Light chain | IR-056 | U.S. Pat. No. 8,858,949 | 5113 |
| | oligomers | variable region | | SEQ ID NO: 756 | |
| AD891 | A beta | Light chain | IR-057 | U.S. Pat. No. 8,858,949 | 5114 |
| | oligomers | variable region | | SEQ ID NO: 772 | |
| AD892 | A beta | Light chain | IR-058 | U.S. Pat. No. 8,858,949 | 5115 |
| | oligomers | variable region | | SEQ ID NO: 788 | |
| AD893 | A beta | Light chain | IR-059 | U.S. Pat. No. 8,858,949 | 5116 |
| | oligomers | variable region | | SEQ ID NO: 804 | |
| AD894 | A beta | Light chain | IR-060 | U.S. Pat. No. 8,858,949 | 5117 |
| | oligomers | variable region | | SEQ ID NO: 820 | |
| AD895 | A beta | Light chain | IR-061 | U.S. Pat. No. 8,858,949 | 5118 |
| | oligomers | variable region | | SEQ ID NO: 836 | |
| AD896 | A beta | Light chain | IR-007 | U.S. Pat. No. 8,858,949 | 5119 |
| | oligomers | variable region | | SEQ ID NO: 84 | |
| AD897 | A beta | Light chain | IR-062 | U.S. Pat. No. 8,858,949 | 5120 |
| | oligomers | variable region | | SEQ ID NO: 852 | |
| AD898 | A beta | Light chain | IR-063 | U.S. Pat. No. 8,858,949 | 5121 |
| | oligomers | variable region | | SEQ ID NO: 868 | |
| AD899 | A beta | Light chain | IR-064 | U.S. Pat. No. 8,858,949 | 5122 |
| | oligomers | variable region | | SEQ ID NO: 884 | |
| AD900 | A beta | Light chain | IR-065 | U.S. Pat. No. 8,858,949 | 5123 |
| | oligomers | variable region | | SEQ ID NO: 900 | |
| AD901 | A beta | Light chain | IR-066 | U.S. Pat. No. 8,858,949 | 5124 |
| | oligomers | variable region | | SEQ ID NO: 916 | |
| AD902 | A beta | Light chain | IR-067 | U.S. Pat. No. 8,858,949 | 5125 |
| | oligomers | variable region | | SEQ ID NO: 932 | |
| AD903 | A beta | Light chain | IR-068 | U.S. Pat. No. 8,858,949 | 5126 |
| | oligomers | variable region | | SEQ ID NO: 948 | |
| AD904 | A beta | Light chain | IR-069 | U.S. Pat. No. 8,858,949 | 5127 |
| | oligomers | variable region | | SEQ ID NO: 964 | |
| AD905 | A beta | Light chain | IR-070 | U.S. Pat. No. 8,858,949 | 5128 |
| | oligomers | variable region | | SEQ ID NO: 980 | |
| AD906 | A beta | Light chain | IR-071 | U.S. Pat. No. 8,858,949 | 5129 |
| | oligomers | variable region | | SEQ ID NO: 996 | |
| AD907 | AB (1-42) | Light chain | Hu8F5VL | US20090232801 | 5130 |
| | Globulomer | variable region | | SEQ ID NO: 105 | |
| AD908 | AB (1-42) | Light chain | TR1.37′CL | US20090232801 | 5131 |
| | Globulomer | variable region | | SEQ ID NO: 106 | |
| AD909 | AB (1-42) | Light chain | Hu8F5VL | US20090232801 | 5132 |
| | Globulomer | variable region | | SEQ ID NO: 112 | |
| AD910 | AB (1-42) | Light chain | 8F5 hum7 VH | US20090232801 | 5133 |
| | Globulomer | variable region | | SEQ ID NO: 2 | |
| AD911 | AB (20-42) | Light chain | VL 5F7hum8 | US20090175847 | 5134 |
| | Globulomer | variable region | | SEQ ID NO: 2 | |
| AD912 | AB (20-42) | Light chain | VL 7C6hum7 | US20090175847 | 5135 |
| | Globulomer | variable region | | SEQ ID NO: 4 | |
| AD913 | ADDL | Light chain | | WO2007050359 | 5136 |
| | | variable region | | SEQ ID NO: 112 | |
| AD914 | ADDL | Light chain | | WO2007050359 | 5137 |
| | | variable region | | SEQ ID NO: 140 | |
| AD915 | amyloid beta | Light chain | | US719576 | 5138 |
| | peptide Aβ | variable region | | SEQ ID NO: 7 | |
| AD916 | amyloid beta | Light chain | | US719576 | 5139 |
| | peptide Aβ | variable region | | SEQ ID NO: 9 | |
| AD917 | amyloid | Light chain | F11G3 | U.S. Pat. No. 9,125,846 | 5140 |
| | oligomers | variable region | | SEQ ID NO: 12 | |
| AD918 | amyloid or | Light chain | Humanized C2 HIV 1 | WO2008061796 | 5141 |
| | amyloid-like | variable region | | SEQ ID NO: 1 | |
| | proteins | | | | |
| AD919 | amyloid protein | Light chain | C2 HuVK | US20100150906 | 5142 |
| | (IGG1 Abeta) | variable region | | SEQ ID NO: 12 | |
| AD920 | amyloid β | Light chain | Fv1E4 | U.S. Pat. No. 8,222,002 | 5143 |
| | peptide | variable region | | SEQ ID NO: 16 | |
| AD921 | amyloid β | Light chain | Fv1E7 | U.S. Pat. No. 8,222,002 | 5144 |
| | peptide | variable region | | SEQ ID NO: 26 | |
| AD922 | amyloid β | Light chain | Fv2A7 | U.S. Pat. No. 8,222,002 | 5145 |
| | peptide | variable region | | SEQ ID NO: 36 | |
| AD923 | amyloid β | Light chain | Fv2A8 | U.S. Pat. No. 8,222,002 | 5146 |
| | peptide | variable region | | SEQ ID NO: 46 | |
| AD924 | amyloid β | Light chain | Fv2B6 | U.S. Pat. No. 8,222,002 | 5147 |
| | peptide | variable region | | SEQ ID NO: 56 | |
| AD925 | amyloid β | Light chain | Fv1E1 | U.S. Pat. No. 8,222,002 | 5148 |
| | peptide | variable region | | SEQ ID NO: 6 | |
| AD926 | amyloid β | Light chain | B7 | U.S. Pat. No. 8,222,002 | 5149 |
| | peptide | variable region | | SEQ ID NO: 66 | |
| AD927 | amyloid β | Light chain | B6 | U.S. Pat. No. 8,222,002 | 5150 |
| | peptide | variable region | | SEQ ID NO: 76 | |
| AD928 | amyloid β | Light chain | F10 | U.S. Pat. No. 8,222,002 | 5151 |
| | peptide | variable region | | SEQ ID NO: 86 | |
| AD929 | amyloid β | Light chain | D1 | U.S. Pat. No. 8,222,002 | 5152 |
| | peptide | variable region | | SEQ ID NO: 96 | |
| AD930 | ApoE-CTD | Light chain | 807B-M0001-B07 | WO2005051998 | 5153 |
| | | variable region | | SEQ ID NO: 150 | |
| AD931 | ApoE-CTD | Light chain | 807B-M0004-A03 | WO2005051998 | 5154 |
| | | variable region | | SEQ ID NO: 151 | |
| AD932 | ApoE-CTD | Light chain | 807B-M0004-A05 | WO2005051998 | 5155 |
| | | variable region | | SEQ ID NO: 152 | |
| AD933 | ApoE-CTD | Light chain | 807B-M0004-C04 | WO2005051998 | 5156 |
| | | variable region | | SEQ ID NO: 153 | |
| AD934 | ApoE-CTD | Light chain | 807B-M0004-C05 | WO2005051998 | 5157 |
| | | variable region | | SEQ ID NO: 154 | |
| AD935 | ApoE-CTD | Light chain | 807B-M0004-F06 | WO2005051998 | 5158 |
| | | variable region | | SEQ ID NO: 155 | |
| AD936 | ApoE-CTD | Light chain | 807B-M0004-F10 | WO2005051998 | 5159 |
| | | variable region | | SEQ ID NO: 156 | |
| AD937 | ApoE-CTD | Light chain | 807B-M0004-H03 | WO2005051998 | 5160 |
| | | variable region | | SEQ ID NO: 157 | |
| AD938 | ApoE-CTD | Light chain | 807B-M0009-C03 | WO2005051998 | 5161 |
| | | variable region | | SEQ ID NO: 158 | |
| AD939 | ApoE-CTD | Light chain | 807B-M0009-F06 | WO2005051998 | 5162 |
| | | variable region | | SEQ ID NO: 159 | |
| AD940 | ApoE-CTD | Light chain | 807B-M0013-A12 | WO2005051998 | 5163 |
| | | variable region | | SEQ ID NO: 160 | |
| AD941 | ApoE-CTD | Light chain | 807B-M0079-D10 | WO2005051998 | 5164 |
| | | variable region | | SEQ ID NO: 161 | |
| AD942 | ApoE-CTD | Light chain | 807B-M0081-F12 | WO2005051998 | 5165 |
| | | variable region | | SEQ ID NO: 162 | |
| AD943 | ApoE-CTD | Light chain | 807B-M0081-H03 | WO2005051998 | 5166 |
| | | variable region | | SEQ ID NO: 163 | |
| AD944 | ApoE-CTD | Light chain | 807B-M0083-E11 | WO2005051998 | 5167 |
| | | variable region | | SEQ ID NO: 164 | |
| AD945 | ApoE-CTD | Light chain | 807A-M0027-E11 | WO2005051998 | 5168 |
| | | variable region | | SEQ ID NO: 42 | |
| AD946 | ApoE-CTD | Light chain | 807A-M0028-B02 | WO2005051998 | 5169 |
| | | variable region | | SEQ ID NO: 43 | |
| AD947 | ApoE-CTD | Light chain | 807A-M0026-F05 | WO2005051998 | 5170 |
| | | variable region | | SEQ ID NO: 44 | |
| AD948 | APP | Light chain | | WO2014151747 | 5171 |
| | | variable region | | SEQ NO 47 | |
| AD949 | APP | Light chain | | WO2014151747 | 5172 |
| | | variable region | | SEQ NO 45 | |
| AD950 | APP | Light chain | | WO2014151747 | 5173 |
| | | variable region | | SEQ NO 49 | |
| AD951 | APP | Light chain | | WO2014151747 | 5174 |
| | | variable region | | SEQ NO 51 | |
| AD952 | Aβ amyloid | Light chain | 15C11 | WO2006066049 | 5175 |
| | | variable region | | SEQ ID NO: 2 | |
| AD953 | Aβ amyloid | Light chain | 9G8 | WO2006066049 | 5176 |
| | | variable region | | SEQ ID NO: 8 | |
| AD954 | Aβ amyloid | Light chain | 266 | WO2006066049 | 5177 |
| | | variable region | | SEQ ID NO: 9 | |
| AD955 | Aβ amyloid | Light chain | 12A1 | WO2006066089 | 5178 |
| | | variable region | | SEQ ID NO: 2 | |
| AD956 | Aβ amyloid | Light chain | 12A1 | WO2006066089 | 5179 |
| | | variable region | | SEQ ID NO: 4 | |
| AD957 | Aβ amyloid | Light chain | humanized 12Al 1 | WO2006066089 | 5180 |
| | | variable region | | SEQ ID NO: 7 | |
| AD958 | Aβ amyloids | Light chain | Humanized 3D6 | U.S. Pat. No. 8,784,810 | 5181 |
| | | variable region | (Bapineuzumb) | SEQ ID NO: 1 | |
| AD959 | Aβ amyloids | Light chain | Humanized 10D5 | U.S. Pat. No. 8,784,810 | 5182 |
| | | variable region | | SEQ ID NO: 28 | |
| AD960 | Aβ amyloids | Light chain | Humanized 3D6 | U.S. Pat. No. 8,784,810 | 5183 |
| | | variable region | (Bapineuzumb), | SEQ ID NO: 3 | |
| | | | version 2 | | |
| AD961 | Aβ amyloids | Light chain | Humanized 12A11 | U.S. Pat. No. 8,784,810 | 5184 |
| | | variable region | | SEQ ID NO: 7 | |
| AD962 | Aβ peptide | Light chain | | U.S. Pat. No. 8,066,999 | 5185 |
| | | variable region | | SEQ ID NO: 1 | |
| AD963 | Aβ polypeptide | Light chain | preferred embodiment | WO2008084402 | 5186 |
| | | variable region | 1, 8, 12 | SEQ ID NO: 145 | |
| AD964 | Aβ polypeptide | Light chain | preferred embodiment | WO2008084402 | 5187 |
| | | variable region | 5, 13 | SEQ ID NO: 146 | |
| AD965 | Aβ polypeptide | Light chain | | WO2008084402 | 5188 |
| | | variable region | | SEQ ID NO: 147 | |
| AD966 | Aβ polypeptide | Light chain | | WO2008084402 | 5189 |
| | | variable region | | SEQ ID NO: 47 | |
| AD967 | Aβ polypeptide | Light chain | | WO2008084402 | 5190 |
| | | variable region | | SEQ ID NO: 48 | |
| AD968 | Aβ polypeptide | Light chain | | WO2008084402 | 5191 |
| | | variable region | | SEQ ID NO: 49 | |
| AD969 | Aβ polypeptide | Light chain | | WO2008084402 | 5192 |
| | | variable region | | SEQ ID NO: 50 | |
| AD970 | Aβ polypeptide | Light chain | preferred embodiment 3 | WO2008084402 | 5193 |
| | | variable region | | SEQ ID NO: 51 | |
| AD971 | Aβ polypeptide | Light chain | preferred embodiment 4 | WO2008084402 | 5194 |
| | | variable region | | SEQ ID NO: 52 | |
| AD972 | Aβ polypeptide | Light chain | preferred embodiment | WO2008084402 | 5195 |
| | | variable region | 2, 6 | SEQ ID NO: 53 | |
| AD973 | Aβ polypeptide | Light chain | preferred embodiment | WO2008084402 | 5196 |
| | | variable region | 9, 10, 11 | SEQ ID NO: 54 | |
| AD974 | Aβ polypeptide | Light chain | preferred embodiment 7 | WO2008084402 | 5197 |
| | | variable region | | SEQ ID NO: 55 | |
| AD975 | Aβ polypeptide | Light chain | | WO2008084402 | 5198 |
| | | variable region | | SEQ ID NO: 56 | |
| AD976 | beta amyloid | Light chain | 12B4 | U.S. Pat. No. 7,256,273 | 5199 |
| | | variable region | | SEQ ID NO: 2 | |
| AD977 | beta amyloid | Light chain | Germline A19 | U.S. Pat. No. 7,256,273 | 5200 |
| | | variable region | | SEQ ID NO: 30 | |
| AD978 | beta amyloid | Light chain | Kabat ID 000333 | U.S. Pat. No. 7,256,273 | 5201 |
| | | variable region | | SEQ ID NO: 32 | |
| AD979 | beta amyloid | Light chain | humanized 12B4 | U.S. Pat. No. 7,256,273 | 5202 |
| | | variable region | | SEQ ID NO: 6 | |
| AD980 | beta amyloid | Light chain | VL A | U.S. Pat. No. 8,323,647 | 5203 |
| | | variable region | | SEQ ID NO: 10 | |
| AD981 | beta amyloid | Light chain | VL B | U.S. Pat. No. 8,323,647 | 5204 |
| | | variable region | | SEQ ID NO: 11 | |
| AD982 | beta amyloid | Light chain | VL C | U.S. Pat. No. 8,323,647 | 5205 |
| | | variable region | | SEQ ID NO: 12 | |
| AD983 | beta amyloid | Light chain | VL D | U.S. Pat. No. 8,323,647 | 5206 |
| | | variable region | | SEQ ID NO: 13 | |
| AD984 | beta amyloid | Light chain | VL E | U.S. Pat. No. 8,323,647 | 5207 |
| | | variable region | | SEQ ID NO: 14 | |
| AD985 | beta amyloid | Light chain | VL F | U.S. Pat. No. 8,323,647 | 5208 |
| | | variable region | | SEQ ID NO: 15 | |
| AD986 | beta amyloid | Light chain | VL G | U.S. Pat. No. 8,323,647 | 5209 |
| | | variable region | | SEQ ID NO: 16 | |
| AD987 | beta amyloid | Light chain | ESBA212 | U.S. Pat. No. 8,323,647 | 5210 |
| | | variable region | | SEQ ID NO: 7 | |
| AD988 | beta amyloid | Light chain | Framework 2.3 | U.S. Pat. No. 8,323,647 | 5211 |
| | | variable region | | SEQ ID NO: 8 | |
| AD989 | beta amyloid | Light chain | 22C4 | U.S. Pat. No. 8,323,647 | 5212 |
| | | variable region | | SEQ ID NO: 9 | |
| AD990 | beta amyloid | Light chain | | US10476265 | 5213 |
| | | variable region | | SEQ ID NO: 7 | |
| AD991 | beta amyloid | Light chain | | US10476265 | 5214 |
| | | variable region | | SEQ ID NO: 8 | |
| AD992 | beta amyloid | Light chain | | US10476265 | 5215 |
| | | variable region | | SEQ ID NO: 9 | |
| AD993 | beta amyloid | Light chain | ACI-11-Ab-9 | US20140199323 | 5216 |
| | | variable region | | SEQ ID NO: 7 | |
| AD994 | beta amyloid | Light chain | ACI-12-Ab-11 | US20140199323 | 5217 |
| | | variable region | | SEQ ID NO: 9 | |
| AD995 | beta amyloid | Light chain | 8C5 | US20150071915 | 5218 |
| | | variable region | | SEQ ID NO: 20 | |
| AD996 | beta amyloid | Light chain | 8F5 | US20150071915 | 5219 |
| | | variable region | | SEQ ID NO: 4 | |
| AD997 | beta amyloid | Light chain | | U.S. Pat. No. 7,189,819 | 5220 |
| | | variable region | | SEQ ID NO: 11 | |
| AD998 | beta amyloid | Light chain | 10D5 | U.S. Pat. No. 7,189,819 | 5221 |
| | | variable region | | SEQ ID NO: 14 | |
| AD999 | beta amyloid | Light chain | m3D6 | U.S. Pat. No. 7,189,819 | 5222 |
| | | variable region | | SEQ ID NO: 2 | |
| AD1000 | beta amyloid | Light chain | humanized 3D6 | U.S. Pat. No. 7,189,819 | 5223 |
| | | variable region | | SEQ ID NO: 5 | |
| AD1001 | beta amyloid | Light chain | Kabal ID 109230 | U.S. Pat. No. 7,189,819 | 5224 |
| | | variable region | | SEQ ID NO: 6 | |
| AD1002 | beta amyloid | Light chain | germline A19 | U.S. Pat. No. 7,189,819 | 5225 |
| | | variable region | antibody | SEQ ID NO: 7 | |
| AD1003 | beta amyloid | Light chain | Bapineuzumab, AAB- | U.S. Pat. No. 8,613,920 | 5226 |
| | | variable region | 001 | SEQ ID NO: 1 | |
| AD1004 | beta amyloid | Light chain | CAA51135 | WO2007113172 | 5227 |
| | peptide | variable region | | SEQ ID NO: 24 | |
| AD1005 | beta amyloid | Light chain | Humanized L1 | WO2007113172 | 5228 |
| | peptide | variable region | | SEQ ID NO: 32 | |
| AD1006 | beta amyloid | Light chain | Mature H2 | WO2007113172 | 5229 |
| | peptide | variable region | | SEQ ID NO: 36 | |
| AD1007 | BETA- | Light chain | NI-101.12 | WO2008081008 | 5230 |
| | AMYLOID | variable region | | SEQ ID NO: 12 | |
| AD1008 | BETA- | Light Chain | NI-101.13 | WO2008081008 | 5231 |
| | AMYLOID | variable region | | SEQ ID NO: 16 | |
| AD1009 | BETA- | Light chain | NI-101.12F6A | WO2008081008 | 5232 |
| | AMYLOID | variable region | | SEQ ID NO: 41 | |
| AD1010 | BETA- | Light chain | NI-101.13A | WO2008081008 | 5233 |
| | AMYLOID | variable region | | SEQ ID NO: 43 | |
| AD1011 | BETA- | Light chain | NI-101.13B | WO2008081008 | 5234 |
| | AMYLOID | variable region | | SEQ ID NO: 45 | |
| AD1012 | BETA- | Light chain | NI-101.10, NI-101.11 | WO2008081008 | 5235 |
| | AMYLOID | variable region | | SEQ ID NO: 8 | |
| AD1013 | DR6 and P75 | Light chain | M73-C04 | WO2010062904 | 5236 |
| | | variable region | | SEQ ID NO: 102 | |
| AD1014 | DR6 and P75 | Light chain | 1P1D6.3 | WO2010062904 | 5237 |
| | | variable region | | SEQ ID NO: 112 | |
| AD1015 | DR6 and P75 | Light chain | M50-H02 | WO2010062904 | 5238 |
| | | variable region | | SEQ ID NO: 12 | |
| AD1016 | DR6 and P75 | Light chain | 1P2F2.1 | WO2010062904 | 5239 |
| | | variable region | | SEQ ID NO: 122 | |
| AD1017 | DR6 and P75 | Light chain | 1P5D10.2 | WO2010062904 | 5240 |
| | | variable region | | SEQ ID NO: 132 | |
| AD1018 | DR6 and P75 | Light chain | M51-H09 | WO2010062904 | 5241 |
| | | variable region | | SEQ ID NO: 22 | |
| AD1019 | DR6 and P75 | Light chain | M53-E04 | WO2010062904 | 5242 |
| | | variable region | | SEQ ID NO: 32 | |
| AD1020 | DR6 and P75 | Light chain | M53-F04 | WO2010062904 | 5243 |
| | | variable region | | SEQ ID NO: 42 | |
| AD1021 | DR6 and P75 | Light chain | M62-B02 | WO2010062904 | 5244 |
| | | variable region | | SEQ ID NO: 52 | |
| AD1022 | DR6 and P75 | Light chain | M63-E10 | WO2010062904 | 5245 |
| | | variable region | | SEQ ID NO: 62 | |
| AD1023 | DR6 and P75 | Light chain | M66-B03 | WO2010062904 | 5246 |
| | | variable region | | SEQ ID NO: 72 | |
| AD1024 | DR6 and P75 | Light chain | M67-G02 | WO2010062904 | 5247 |
| | | variable region | | SEQ ID NO: 82 | |
| AD1025 | DR6 and P75 | Light chain | M72-F03 | WO2010062904 | 5248 |
| | | variable region | | SEQ ID NO: 92 | |
| AD1026 | IOD5 | Light chain | | WO2002088307 | 5249 |
| | | variable region | | SEQ ID NO: 11 | |
| AD1027 | IOD5 | Light chain | | WO2002088307 | 5250 |
| | | variable region | | SEQ ID NO: 7 | |
| AD1028 | IOD5 | Light chain | | WO2002088307 | 5251 |
| | | variable region | | SEQ ID NO: 9 | |
| AD1029 | LPG | Light chain | #7 | U.S. Pat. No. 8,591,902 | 5252 |
| | (lysophosphatidylglucoside) | variable region | | SEQ ID NO: 17 | |
| AD1030 | LPG | Light chain | #15 | U.S. Pat. No. 8,591,902 | 5253 |
| | (lysophosphatidylglucoside) | variable region | | SEQ ID NO: 7 | |
| AD1031 | MAG | Light chain | | U.S. Pat. No. 8,071,731 | 5254 |
| | | variable region | | SEQ ID NO: 16 | |
| AD1032 | MAG | Light chain | | U.S. Pat. No. 8,071,731 | 5255 |
| | | variable region | | SEQ ID NO: 17 | |
| AD1033 | MAG | Light chain | | U.S. Pat. No. 8,071,731 | 5256 |
| | | variable region | | SEQ ID NO: 18 | |
| AD1034 | MAG | Light chain | | U.S. Pat. No. 8,071,731 | 5257 |
| | | variable region | | SEQ ID NO: 19 | |
| AD1035 | MAI (myelin | Light chain | | WO2013158748 | 5258 |
| | associated | variable region | | SEQ ID NO: 11 | |
| | inhibitor) | | | | |
| AD1036 | MAI (myelin | Light chain | | WO2013158748 | 5259 |
| | associated | variable region | | SEQ ID NO: 27 | |
| | inhibitor) | | | | |
| AD1037 | NMDA | Light chain | | EP2805972 SEQ | 5260 |
| | | variable region | | ID NO: 44 | |
| AD1038 | NOGO | Light chain | H1L6, H5L6, H6L6, | US20140147435 | 5261 |
| | | variable region | H14L6, H15L6, | SEQ ID NO: 19 | |
| | | | H16L6, H17L6, | | |
| | | | H18L6, H19L6, | | |
| | | | H20L6, H21L6, | | |
| | | | H22L6, H23L6, | | |
| | | | H24L6, H25L6, | | |
| | | | H700L6 | | |
| AD1039 | NOGO | Light chain | H1L13, H5L13, | US20140147435 | 5262 |
| | | variable region | H6L13, H14L13, | SEQ ID NO: 20 | |
| | | | H15L13, H16L13, | | |
| | | | H17L13, H18L13, | | |
| | | | H19L13, H20L13, | | |
| | | | H21L13, H22L13, | | |
| | | | H23L13, H24L13, | | |
| | | | H25L13, H700L13 | | |
| AD1040 | NOGO | Light chain | H1L14, H5L14, | US20140147435 | 5263 |
| | | variable region | H6L14, H14L14, | SEQ ID NO: 21 | |
| | | | H15L14, H16L14, | | |
| | | | H17L14, H18L14, | | |
| | | | H19L14, H20L14, | | |
| | | | H21L14, H22L14, | | |
| | | | H23L14, H24L14, | | |
| | | | H25L14, H700L14 | | |
| AD1041 | NOGO | Light chain | H1L15, H5L15, | US20140147435 | 5264 |
| | | variable region | H6L15, H14L15, | SEQ ID NO: 22 | |
| | | | H15L15, H16115, | | |
| | | | H17L15, H18L15, | | |
| | | | H19L15, H20L15, | | |
| | | | H21L15, H22L15, | | |
| | | | H23L15, H24L15, | | |
| | | | H25L15, H700L15 | | |
| AD1042 | NOGO | Light chain | H1L16, H5L16, | US20140147435 | 5265 |
| | | variable region | H6L16, H14L16, | SEQ ID NO: 23 | |
| | | | H15L16, H16L16, | | |
| | | | H17L16, H18L16, | | |
| | | | H19L16, H20L16, | | |
| | | | H21L16, H22L16, | | |
| | | | H23L16, H24L16, | | |
| | | | H25L16, H700L16 | | |
| AD1043 | NOGO | Light chain | H1L17, H5L17, | US20140147435 | 5266 |
| | | variable region | H6L17, H14L17, | SEQ ID NO: 24 | |
| | | | H15L17, H16L17, | | |
| | | | H17L17, H18L17, | | |
| | | | H19L17, H20L17, | | |
| | | | H21L17, H22L17, | | |
| | | | H23L17, H24L17, | | |
| | | | H25L17, H700L17 | | |
| AD1044 | NOGO | Light chain | H1L18, H5L18, | US20140147435 | 5267 |
| | | variable region | H6L18, H14L18, | SEQ ID NO: 25 | |
| | | | H15L18, H16L18, | | |
| | | | H17L18, H18L18, | | |
| | | | H19L18, H20L18, | | |
| | | | H21L18, H22L18, | | |
| | | | H23L18, H24L18, | | |
| | | | H25L18, H700L18 | | |
| AD1045 | NOGO | Light chain | H5L11, H6L11, | US20140147435 | 5268 |
| | | variable region | H14L11, H15L11, | SEQ ID NO: 78 | |
| | | | H16L11, H17L11, | | |
| | | | H18L11, H19L11, | | |
| | | | H20L11, H21L11, | | |
| | | | H22L11, H23L11, | | |
| | | | H24L11, H25L11, | | |
| | | | H700L11 | | |
| AD1046 | Nogo-66 | Light chain | Antibody clone 50 | US20140065155 | 5269 |
| | | variable region | | SEQ ID NO: 4 | |
| AD1047 | Nogo-66 | Light chain | Antibody clone 51 | US20140065155 | 5270 |
| | | variable region | | SEQ ID NO: 6 | |
| AD1048 | NogoA/NiG | Light chain | 6A3-Ig4 | WO2009056509 | 5271 |
| | | variable region | | SEQ ID NO: 25 | |
| AD1049 | NogoA/NiG | Light chain | 6A3-IgG1 | WO2009056509 | 5272 |
| | | variable region | | SEQ ID NO: 5 | |
| AD1050 | N-terminal | Light chain | Antibody TeiA 1.6 | US20110059092 | 5273 |
| | region of Aβ8- | variable region | (Secreted by | SEQ ID NO: 1 | |
| | x peptide | | Hybridoma IGH521) | | |
| AD1051 | N-terminal | Light chain | Antibody TeiA 1.7 | US20110059092 | 5274 |
| | region of Aβ8- | variable region | (Secreted by | SEQ ID NO: 3 | |
| | x peptide | | Hybridoma IGH522) | | |
| AD1052 | N-terminal | Light chain | Antibody TeiA 1.8 | US20110059092 | 5275 |
| | region of Aβ8- | variable region | (Secreted by | SEQ ID NO: 5 | |
| | x peptide | | Hybridoma IGH523) | | |
| AD1053 | N-terminal | Light chain | Antibody TeiA 2b.6 | US20110059092 | 5276 |
| | region of Aβ8- | variable region | (Secreted by | SEQ ID NO: 7 | |
| | x peptide | | Hybridoma IGH524) | | |
| AD1054 | N-terminal | Light chain | Antibody TeiA 1.1 | US20110059092 | 5277 |
| | region of Aβ8- | variable region | (Secreted by | SEQ ID NO: 9 | |
| | x peptide | | Hybridoma IGH525) | | |
| AD1055 | oligomers of N- | Light chain | 9D5 | U.S. Pat. No. 8,795,664 | 5278 |
| | terminal | variable region | | SEQ ID NO: 28 | |
| | truncated Aβ | | | | |
| AD1056 | oligomers of N- | Light chain | 8C4 | U.S. Pat. No. 8,795,664 | 5279 |
| | terminal | variable region | | SEQ ID NO: 32 | |
| | truncated Aβ | | | | |
| AD1057 | PrPC and/or | Light chain | | US20150166668 | 5280 |
| | PrPSc | variable region | | SEQ ID NO: 7 | |
| AD1058 | pyroglutamated | Light chain | | WO2012136552 | 5281 |
| | A β | variable region | | SEQ ID NO: 11 | |
| AD1059 | pyroglutamated | Light chain | | WO2012136552 | 5282 |
| | A β | variable region | | SEQ ID NO: 27 | |
| AD1060 | pyroglutamated | Light chain | | WO2012136552 | 5283 |
| | A β | variable region | | SEQ ID NO: 31 | |
| AD1061 | pyroglutamated | Light chain | | WO2012136552 | 5284 |
| | A β | variable region | | SEQ ID NO: 7 | |
| AD1062 | RGM A | Light chain | 5F9.1-GL, 5F9.1-GL, | US20150183871 | 5285 |
| | | variable region | 5F9.1-GL, 5F9.1-GL, | SEQ ID NO: 44 | |
| | | | 5F9.1-GL, 5F9.1-GL, | | |
| | | | 5F9.1-GL, 5F9.1-GL, | | |
| | | | 5F9.1-GL, 5F9.1-GL, | | |
| | | | h5F9.4, h5F9.11, | | |
| | | | h5F9.12 | | |
| AD1063 | RGM A | Light chain | 5F9.2-GL, 5F9.2-GL, | US20150183871 | 5286 |
| | | variable region | 5F9.2-GL, 5F9.2-GL, | SEQ ID NO: 45 | |
| | | | 5F9.2-GL, 5F9.2-GL, | | |
| | | | 5F9.2-GL, 5F9.2-GL, | | |
| | | | 5F9.2-GL, 5F9.2-GL, | | |
| | | | h5F9.5, h5F9.19, | | |
| | | | h5F9.20 | | |
| AD1064 | RGM A | Light chain | 5F9.3-GL, 5F9.3-GL, | US20150183871 | 5287 |
| | | variable region | 5F9.3-GL, 5F9.3-GL, | SEQ ID NO: 46 | |
| | | | 5F9.3-GL, 5F9.3-GL, | | |
| | | | 5F9.3-GL, 5F9.3-GL, | | |
| | | | 5F9.3-GL, 5F9.3-GL, | | |
| | | | h5F9.6, h5F9.21, | | |
| | | | h5F9.22 | | |
| AD1065 | RGM A | Light chain | h5F9.5, h5F9.6, | US20150183871 | 5288 |
| | | variable region | h5F9,7, h5F9,8, | SEQ ID NO: 48 | |
| | | | h5F9.9, h5F9.10 | | |
| AD1066 | RGM A | Light chain | h5F9.11, | US20150183871 | 5289 |
| | | variable region | h5F9.19, h5F9.21 | SEQ ID NO: 49 | |
| AD1067 | RGM A | Light chain | h5F9.1.2, h5F9.20, | US20150183871 | 5290 |
| | | variable region | h5F9.22, h5F9.23, | SEQ ID NO:. 50 | |
| | | | h5F9.25, h5F9.25, | | |
| | | | h5F9.26 | | |
| AD1068 | RGM A | Light chain | h5F9.1, h5F9.7, | US20150183871 | 5291 |
| | | variable region | h5F9.23 | SEQ ID NO: 51 | |
| AD1069 | RGM A | Light chain | h5F9.2, h5F9.8, | US20150183871 | 5292 |
| | | variable region | h5F9.25 | SEQ ID NO: 52 | |
| AD1070 | RGMa | Light chain | AE12-1 | US20140023659 | 5293 |
| | | variable region | | SEQ ID NO: 5 | |
| AD1071 | RGMa | Light chain | AE12-7 | US20140023659 | 5294 |
| | | variable region | | SEQ ID NO: 53 | |
| AD1072 | RGMa | Light chain | AE12-8 | US20140023659 | 5295 |
| | | variable region | | SEQ ID NO: 61 | |
| AD1073 | RGMa | Light chain | AE12-13 | US20140023659 | 5296 |
| | | variable region | | SEQ ID NO: 95 | |
| AD1074 | RGMa | Light chain | AE12-15 | US20140023659 | 5297 |
| | | variable region | | SEQ ID NO: 103 | |
| AD1075 | RGMa | Light chain | AE12-20 | US20140023659 | 5298 |
| | | variable region | | SEQ ID NO: 111 | |
| AD1076 | RGMa | Light chain | AE12-21 | US20140023659 | 5299 |
| | | variable region | | SEQ ID NO: 119 | |
| AD1077 | RGMa | Light chain | AE12-23 | US20140023659 | 5300 |
| | | variable region | | SEQ ID NO: 127 | |
| AD1078 | RGMa | Light chain | AE12-2 | US20140023659 | 5301 |
| | | variable region | | SEQ ID ID: 13 | |
| AD1079 | RGMa | Light chain | AE12-24 | US20140023659 | 5302 |
| | | variable region | | SEQ ID NO: 135 | |
| AD1080 | RGMa | Light chain | AE12-3 | US20140023659 | 5303 |
| | | variable region | | SEQ ID NO: 21 | |
| AD1081 | RGMa | Light chain | AE12-4 | US20140023659 | 5304 |
| | | variable region | | SEQ ID NO: 29 | |
| AD1082 | RGMa | Light chain | AE12-5 | US20140023659 | 5305 |
| | | variable region | | SEQ ID NO: 37 | |
| AD1083 | RGMa | Light chain | AE12-6 | US20140023659 | 5306 |
| | | variable region | | SEQ ID NO: 45 | |
| AD1084 | tau | Light chain | NI-105.4E4 | US20150252102 | 5307 |
| | | variable region | | SEQ ID NO: 11 | |
| AD1085 | tau | Light chain | NI-105.4B2 | US20150252102 | 5308 |
| | | variable region | | SEQ ID NO: 15 | |
| AD1086 | tau | Light chain | NI-105.4A3 | US20150252102 | 5309 |
| | | variable region | | SEQ ID NO: 19 | |
| AD1087 | tau | Light chain | | WO2013041962 | 5310 |
| | | variable region | | SEQ ID NO: 141 | |
| AD1088 | tau | Light chain | | WO2013041962 | 5311 |
| | | variable region | | SEQ ID NO: 142 | |
| AD1089 | tau | Light chain | | WO2013041962 | 5312 |
| | | variable region | | SEQ ID NO: 143 | |
| AD1090 | tau | Light chain | | WO2013041962 | 5313 |
| | | variable region | | SEQ ID NO: 150 | |
| AD1091 | tau | Light chain | | WO2013041962 | 5314 |
| | | variable region | | SEQ ID NO: 152 | |
| AD1092 | tau | Light chain | | WO2013041962 | 5315 |
| | | variable region | | SEQ ID NO: 153 | |
| AD1093 | tau | Light chain | | WO2014100600 | 5316 |
| | | variable region | | SEQID NO: 221 | |
| AD1094 | tau | Light chain | | WO2014100600 | 5317 |
| | | variable region | | SEQID NO: 222 | |
| AD1095 | tau | Light chain | NI-105.17C1 | WO2014100600 | 5318 |
| | | variable region | | SEQID NO: 46 | |
| AD1096 | tau | Light chain | NI-105.6C5 | WO2014100600 | 5319 |
| | | variable region | | SEQID NO: 49 | |
| AD1097 | tau | Light chain | NI-105.29G10 | WO2014100600 | 5320 |
| | | variable region | | SEQID NO: 51 | |
| AD1098 | tau | Light chain | NI-105.6L9 | WO2014100600 | 5321 |
| | | variable region | | SEQID NO: 53 | |
| AD1099 | tau | Light chain | NI-105.40E8 | WO2014100600 | 5322 |
| | | variable region | | SEQID NO: 55 | |
| AD1100 | tau | Light chain | NI-105.48E5 | WO2014100600 | 5323 |
| | | variable region | | SEQID NO: 57 | |
| AD1101 | tau | Light chain | NI-105.6E3 | WO2014100600 | 5324 |
| | | variable region | | SEQID NO: 59 | |
| AD1102 | tau | Light chain | NI-105.22E1 | WO2014100600 | 5325 |
| | | variable region | | SEQID NO: 61 | |
| AD1103 | tau | Light chain | | WO2014100600 | 5326 |
| | | variable region | | SEQID NO: 63 | |
| AD1104 | tau | Light chain | NI-105.26B12 | WO2014100600 | 5327 |
| | | variable region | | SEQID NO: 64 | |
| AD1105 | tau | Light chain | NI-105.12E12 | WO2014100600 | 5328 |
| | | variable region | | SEQID NO: 66 | |
| AD1106 | tau | Light chain | NI-105.60E7 | WO2014100600 | 5329 |
| | | variable region | | SEQID NO: 68 | |
| AD1107 | tau | Light chain | NI-105,14E2 | WO2014100600 | 5330 |
| | | variable region | | SEQID NO: 70 | |
| AD1108 | tau | Light chain | NI-105.39E2 | WO2014100600 | 5331 |
| | | variable region | | SEQID NO: 72 | |
| AD1109 | tau | Light chain | NI-105.19C6 | WO2014100600 | 5332 |
| | | variable region | | SEQID NO: 74 | |
| AD1110 | tau | Light chain | | WO2014100600 | 5333 |
| | | variable region | | SEQID NO: 77 | |
| AD1111 | tau | Light chain | NI-105.9C4 | WO2014100600 | 5334 |
| | | variable region | | SEQID NO: 78 | |
| AD1112 | tau | Light chain | IPN002 variant 1 | U.S. Pat. No. 8,926,974 | 5335 |
| | | variable region | | SEQ ID NO: 40 | |
| AD1113 | tau | Light chain | IPN002 variant 2 | U.S. Pat. No. 8,926,974 | 5336 |
| | | variable region | | SEQ ID NO: 41 | |
| AD1114 | tau | Light chain | IPN002 variant 3 | U.S. Pat. No. 8,926,974 | 5337 |
| | | variable region | | SEQ ID NO: 42 | |
| AD1115 | tau | Light chain | IPIN002 variant 4 | U.S. Pat. No. 8,926,974 | 5338 |
| | | variable region | | SEQ ID NO: 43 | |
| AD1116 | tau | Light chain | PT1 | US20150307600 | 5339 |
| | | variable region | | SEQ ID NO: 36 | |
| AD1117 | tau | Light chain | PT3 | US20150307600 | 5340 |
| | | variable region | | SEQ ID NO: 38 | |
| AD1118 | tau | Light chain | | U.S. Pat. No. 9,304,138 | 5341 |
| | | variable region | | SEQ ID NO: 6 | |
| AD1119 | tau | Light chain | | U.S. Pat. No. 9,304,138 | 5342 |
| | | variable region | | SEQ ID NO: 7 | |
| AD1120 | tau | Light chain | | U.S. Pat. No. 9,304,138 | 5343 |
| | | variable region | | SEQ ID NO: 8 | |
| AD1121 | tau | Light chain | | U.S. Pat. No. 9,304,138 | 5344 |
| | | variable region | | SEQ ID NO: 9 | |
| AD1122 | tau | Light chain | | U.S. Pat. No. 9,304,138 | 5345 |
| | | variable region | | SEQ ID NO: 10 | |
| AD1123 | tau | Light chain | | U.S. Pat. No. 9,304,138 | 5346 |
| | | variable region | | SEQ ID NO: 11 | |
| AD1124 | tau | Light chain | | U.S. Pat. No. 9,304,138 | 5347 |
| | | variable region | | SEQ ID NO: 69 | |
| AD1125 | tau | Light chain | | U.S. Pat. No. 9,304,138 | 5348 |
| | | variable region | | SEQ ID NO: 77 | |
| AD1126 | tau | Light chain | | U.S. Pat. No. 9,304,138 | 5349 |
| | | variable region | | SEQ ID NO: 92 | |
| AD1127 | tau | Light chain | | U.S. Pat. No. 9,304,138 | 5350 |
| | | variable region | | SEQ ID NO: 97 | |
| AD1128 | tau | Light chain | | U.S. Pat. No. 9,304,138 | 5351 |
| | | variable region | | SEQ ID NO: 105 | |
| AD1129 | tau | Light chain | | U.S. Pat. No. 9,304,138 | 5352 |
| | | variable region | | SEQ ID NO: 116 | |
| AD1130 | tau | Light chain | | U.S. Pat. No. 9,304,138 | 5353 |
| | | variable region | | SEQ ID NO: 118 | |
| AD1131 | tau | Light chain | hACl-36-3A8-Ab1 | US20150175682 | 5354 |
| | | variable region | | SEQ ID NO: 8 | |
| AD1132 | tau | Light chain | hACl-36-2B6-Ab1 | US20150175682 | 5355 |
| | | variable region | | SEQ ID NO: 9 | |
| AD1133 | tau | Light chain | ADx210 | US20140161875 | 5356 |
| | | variable region | | SEQ ID NO: 16 | |
| AD1134 | tau | Light chain | ADx210 isoform | US20140161875 | 5357 |
| | | variable region | | SEQ ID NO: 18 | |
| AD1135 | tau | Light chain | ADx215 | US20140161875 | 5358 |
| | | variable region | | SEQ ID NO: 26 | |
| AD1136 | tau antigen | Light chain | ADx202 | WO2015004163 | 5359 |
| | | variable region | | SEQ ID NO: 9 | |
| AD1137 | tau ps 422 | Light chain | antibody Mab2.10.3 | US20110059093 | 5360 |
| | | variable region | | SEQ ID NO: 1 | |
| AD1138 | tau ps 422 | Light chain | Mab 005 | US20110059093 | 5361 |
| | | variable region | | SEQ ID NO: 26 | |
| AD1139 | tau ps 422 | Light chain | Mab 019 | US20110059093 | 5362 |
| | | variable region | | SEQ ID NO: 34 | |
| AD1140 | tau ps 422 | Light chain | Mab 020 | US20110059093 | 5363 |
| | | variable region | | SEQ ID NO: 42 | |
| AD1141 | tau ps 422 | Light chain | Mab 085 | US20110059093 | 5364 |
| | | variable region | | SEQ ID NO: 50 | |
| AD1142 | tau ps 422 | Light chain | Mab 086 | US20110059093 | 5365 |
| | | variable region | | SEQ ID NO: 58 | |
| AD1143 | tau ps 422 | Light chain | Mab 097 | US20110059093 | 5366 |
| | | variable region | | SEQ ID NO: 66 | |
| AD1144 | TrkA | Light chain | Hu1lo | WO2009098238 | 5367 |
| | | variable region | | SEQ ID NO: 18 | |
| AD1145 | TrkA | Light chain | 3-23*01 | WO2009098238 | 5368 |
| | | variable region | | SEQ ID NO: 19 | |
| AD1146 | TrkA | Light chain | JH4 | WO2009098238 | 5369 |
| | | variable region | | SEQ ID NO: 20 | |
| AD1147 | TrkA | Light chain | L6*01 | WO2009098238 | 5370 |
| | | variable region | | SEQ ID NO: 21 | |
| AD1148 | TrkA | Light chain | JK1 | WO2009098238 | 5371 |
| | | variable region | | SEQ ID NO: 22 | |
| AD1149 | TrkA | Light chain | BXhVH5VLl N297A i | WO2009098238 | 5372 |
| | | variable region | | SEQ ID NO: 23 | |
| AD1150 | NOGO | Light chain | 2A10 construct | WO2007003421 | 5373 |
| | | variable region | | SEQ ID NO: 78 | |
| | | humanized | | | |
| | | construct L11 | | | |
| AD1151 | NOGO | Light chain | 2A10 construct | WO2007003421 | 5374 |
| | | variable region | | SEQ ID NO: 20 | |
| | | humanized | | | |
| | | construct L13 | | | |
| AD1152 | NOGO | Light chain | 2A10 construct | WO2007003421 | 5375 |
| | | variable region | | SEQ ID NO: 21 | |
| | | humanized | | | |
| | | construct L14 | | | |
| AD1153 | NOGO | Light chain | 2A10 construct | WO2007003421 | 5376 |
| | | variable region | | SEQ ID NO: 22 | |
| | | humanized | | | |
| | | construct L15 | | | |
| AD1154 | NOGO | Light chain | 2A10 construct | WO2007003421 | 5377 |
| | | variable region | | SEQ ID NO: 23 | |
| | | humanized | | | |
| | | construct L16 | | | |
| AD1155 | NOGO | Light chain | 2A10 construct | WO2007003421 | 5378 |
| | | variable region | | SEQ ID NO: 24 | |
| | | humanized | | | |
| | | construct L17 | | | |
| AD1156 | NOGO | Light chain | 2A10 construct | WO2007003421 | 5379 |
| | | variable region | | SEQ ID NO: 25 | |
| | | humanized | | | |
| | | construct L18 | | | |
| AD1157 | NOGO | Light chain | 2A10 construct | WO2007003421 | 5380 |
| | | variable region | | SEQ ID NO: 19 | |
| | | humanized | | | |
| | | construct L6 | | | |
| AD1158 | beta A4 | Light chain with | Antibody A | WO2007068429 | 5381 |
| | peptide/Alpha | leader sequence | | SEQ ID NO: 28 | |
| | beta 9 | | | | |
| AD1159 | Aβ amyloid | Light chain, | | WO2006066089 | 5382 |
| | | consensus | | SEQ ID NO: 38 | |
| AD1160 | Aβ amyloid | Light chain, | | WO2006066089 | 5383 |
| | | consensus | | SEQ ID NO: 39 | |
| AD1161 | Aβ amyloid | Light chain, | | WO2006066089 | 5384 |
| | | consensus | | SEQ ID NO: 40 | |
| AD1162 | Aβ amyloid | Light chain, | | WO2006066089 | 5385 |
| | | consensus | | SEQ ID NO: 41 | |
| AD1163 | Aβ amyloid | Light chain, | | WO2006066089 | 5386 |
| | | consensus | | SEQ ID NO: 42 | |
| AD1164 | Aβ amyloid | Light chain, | | WO2006066089 | 5387 |
| | | consensus | | SEQ ID NO: 43 | |
| AD1165 | Aβ amyloid | Light chain, | | WO2006066089 | 5388 |
| | | consensus | | SEQ ID NO: 44 | |
| AD1166 | Aβ amyloid | Light chain, | | WO2006066089 | 5389 |
| | | consensus | | SEQ ID NO: 45 | |
| AD1167 | Aβ amyloid | Light chain, | | WO2006066089 | 5390 |
| | | consensus | | SEQ ID NO: 46 | |
| AD1168 | Aβ amyloid | Light chain, | | WO2006066089 | 5391 |
| | | consensus | | SEQ ID NO: 47 | |
| AD1169 | Aβ amyloid | Light chain, | | WO2006066089 | 5392 |
| | | consensus | | SEQ ID NO: 48 | |
| AD1170 | Aβ amyloid | Light chain, | | WO2006066089 | 5393 |
| | | consensus | | SEQ ID NO: 49 | |
| AD1171 | Aβ amyloid | Light chain, | | WO2006066089 | 5394 |
| | | consensus | | SEQ ID NO: 50 | |
| AD1172 | Aβ amyloid | Light chain, | | WO2006066089 | 5395 |
| | | consensus | | SEQ ID NO: 51 | |
| AD1173 | PrPC and/or | scFv | | U.S. Pat. No. 8,852,587 | 5396 |
| | PrPSc | | | SEQ ID NO: 6 | |
| AD1174 | beta amyloid | scFv | RCK37 | U.S. Pat. No. 8,221,750 | 5397 |
| | | | | SEQ ID NO: 6 | |
| AD1175 | beta amyloid | scFv | RCK22 | U.S. Pat. No. 8,221,750 | 5398 |
| | | | | SEQ ID NO: 8 | |
| AD1176 | PrP | | ICSM181c | US20140294844 | 5399 |
| | | | | SEQ ID NO: 6 | |
| AD1177 | PrPC and/or | | | U.S. Pat. No. 8,852,587 | 5400 |
| | PrPSc | | | SEQ ID NO: 3 | |
| AD1178 | tau | | | US20140302046 | 5401 |
| | | | | SEQ ID NO: 103 | |

### Huntington's Disease Antibodies

In some embodiments, the payload region of the viral particle comprises one or more nucleic acid sequences encoding one or more of the Huntington's Disease payload antibody polypeptides listed in Table 5 (HD1-HD245; SEQ ID NO: 5402-5646).

TABLE 5
Huntington's Disease Antibodies

| Antibody | | | | Reference | SEQ ID |
|---|---|---|---|---|---|
| No. | Target | Description | Antibody Name | Information | NO |
| HD1 | amyloid | consensus | M13 g3p, fd g3p, f1 | US20150376239 | 5402 |
| | proteins | sequence | g3p | SEQ ID NO: 4 | |
| HD2 | amyloid | consensus | 12-2 g3p, Ike g3p | US20150376239 | 5403 |
| | proteins | sequence | | SEQ ID NO: 7 | |
| HD3 | 118-126 of α- | constant region | IgG1 | US20150259404 | 5404 |
| | synuclein | | | SEQ ID NO: 38 | |
| HD4 | amyloid | Fusion protein | M13 g3p | US20150376239 | 5405 |
| | proteins | | | SEQ ID NO: 1 | |
| HD5 | amyloid | Fusion protein | Construct 5 | US20150376239 | 5406 |
| | proteins | | | SEQ ID NO: 11 | |
| HD6 | amyloid | Fusion protein | Construct 6 | US20150376239 | 5407 |
| | proteins | | | SEQ ID NO: 13 | |
| HD7 | amyloid | Fusion protein | fd N2 | US20150376239 | 5408 |
| | proteins | | | SEQ ID NO: 14 | |
| HD8 | amyloid | Fusion protein | f1 N2 | US20150376239 | 5409 |
| | proteins | | | SEQ ID NO: 15 | |
| HD9 | amyloid | Fusion protein | M13 N2 | US20150376239 | 5410 |
| | proteins | | | SEQ ID NO: 16 | |
| HD10 | amyloid | Fusion protein | Ike N2 | US20150376239 | 5411 |
| | proteins | | | SEQ ID NO: 17 | |
| HD11 | amyloid | Fusion protein | 12-2 N2 | US20150376239 | 5412 |
| | proteins | | | SEQ ID NO: 18 | |
| HD12 | amyloid | Fusion protein | If1 N2 | US20150376239 | 5413 |
| | proteins | | | SEQ ID NO: 19 | |
| HD13 | amyloid | Fusion protein | fd g3p | US20150376239 | 5414 |
| | proteins | | | SEQ ID NO: 2 | |
| HD14 | amyloid | Fusion protein | Construct 3 | US20150376239 | 5415 |
| | proteins | | | SEQ ID NO: 20 | |
| HD15 | amyloid | Fusion protein | Construct 3m g3p | US20150376239 | 5416 |
| | proteins | | portion | SEQ ID NO: 24 | |
| HD16 | amyloid | Fusion protein | If1 g3p | US20150376239 | 5417 |
| | proteins | | | SEQ ID NO: 29 | |
| HD17 | amyloid | Fusion protein | f1 g3p | US20150376239 | 5418 |
| | proteins | | | SEQ ID NO: 3 | |
| HD18 | amyloid | Fusion protein | fd g3p | US20150376239 | 5419 |
| | proteins | | | SEQ ID NO: 30 | |
| HD19 | amyloid | Fusion protein | Construct 8, rs-g3p | US20150376239 | 5420 |
| | proteins | | (If1-N1N2)-hlgG1- | SEQ ID NO: 31 | |
| | | | Fc | | |
| HD20 | amyloid | Fusion protein | I2-2 g3p | US20150376239 | 5421 |
| | proteins | | | SEQ ID NO: 5 | |
| HD21 | amyloid | Fusion protein | Ike g3p | US20150376239 | 5422 |
| | proteins | | | SEQ ID NO: 6 | |
| HD22 | amyloid | Fusion protein | If1 g3p | US20150376239 | 5423 |
| | proteins | | | SEQ ID NO: 8 | |
| HD23 | amyloid | Fusion protein | Construct 4 | US20150376239 | 5424 |
| | proteins | | | SEQ ID NO: 9 | |
| HD24 | amyloids | Heavy chain | #118 | WO2010012004 | 5425 |
| | | | | SEQ ID NO: 11 | |
| HD25 | amyloids | Heavy chain | #121 | WO2010012004 | 5426 |
| | | | | SEQ ID NO: 13 | |
| HD26 | amyloids | Heavy chain | #204 | WO2010012004 | 5427 |
| | | | | SEQ ID NO: 16 | |
| HD27 | amyloids | Heavy chain | #205 | WO20100I2004 | 5428 |
| | | | | SEQ ID NO: 18 | |
| HD28 | NOGO | Heavy chain | H6L13 FL | US20140147435 | 5429 |
| | | | | SEQ ID NO: 27 | |
| HD29 | NOGO | Heavy chain | H16L16 FL, H16L18 | US20140147435 | 5430 |
| | | | FL | SEQ ID NO: 31 | |
| HD30 | NOGO | Heavy chain | H18L16 FL | US20140147435 | 5431 |
| | | | | SEQ ID NO: 33 | |
| HD31 | NOGO | Heavy chain | H19L13 FL, H19L16 | US20140147435 | 5432 |
| | | | FL, H19L18 FL | SEQ ID NO: 92 | |
| HD32 | NOGO | Heavy chain | H20L13 FL, H20L16 | US20140147435 | 5433 |
| | | | FL, H20L18 FL | SEQ ID NO: 93 | |
| HD33 | NOGO | Heavy chain | H21L13 FL, H21L16 | US20140147435 | 5434 |
| | | | FL, H21L 18 FL | SEQ ID NO: 94 | |
| HD34 | NOGO | Heavy chain | H25L13 FL, H25L16 | US20140147435 | 5435 |
| | | | FL, H25L 18 FL | SEQ ID NO: 98 | |
| HD35 | Nogo receptor- | Heavy chain | 5B10 | US20090215691 | 5436 |
| | 1 | | | SEQ ID NO: 16 | |
| HD36 | Nogo receptor- | Heavy chain | 5B10 | US20090215691 | 5437 |
| | 1 | | | SEQ ID NO: 18 | |
| HD37 | trk-C (NT-3 | Heavy chain | 2250 | U.S. Pat. No. | 5438 |
| | trkC ligand) | | | 7,615,383 | |
| | | | | SEQ ID NO: 42 | |
| HD38 | trk-C (NT-3 | Heavy chain | 2253 | U.S. Pat. No. | 5439 |
| | trkC ligand) | | | 7,615,383 | |
| | | | | SEQ ID NO: 43 | |
| HD39 | trk-C (NT-3 | Heavy chain | 2256 | U.S. Pat. No. | 5440 |
| | trkC ligand) | | | 7,615,383 | |
| | | | | SEQ ID NO: 44 | |
| HD40 | trk-C (NT-3 | Heavy chain | 6.1.2 | U.S. Pat. No. | 5441 |
| | trkC ligand) | | | 7,615,383 | |
| | | | | SEQ ID NO: 45 | |
| HD41 | trk-C (NT-3 | Heavy chain | 6.4.1 | U.S. Pat. No. | 5442 |
| | tkC ligand) | | | 7,615,383 | |
| | | | | SEQ ID NO: 46 | |
| HD42 | trk-C (NT-3 | Heavy chain | 2345 | U.S. Pat. No. | 5443 |
| | trkC ligand) | | | 7,615,383 | |
| | | | | SEQ ID NO: 47 | |
| HD43 | trk-C (NT-3 | Heavy chain | 2349 | U.S. Pat. No. | 5444 |
| | trkC ligand) | | | 7,615,383 | |
| | | | | SEQ ID NO: 48 | |
| HD44 | many | Heavy chain fusion | H19L13, H19L16, | U.S. Pat. No. | 5445 |
| | | protein | H19L18, H19L14, | 8,053,569 | |
| | | | H19L15, H19L17, | SEQ ID NO: 25 | |
| | | | H19L6, H19L11 | | |
| HD45 | many | Heavy chain fusion | H20L13, H20L16, | U.S. Pat. No. | 5446 |
| | | protein | H20L18, H20L14, | 8,053,569 | |
| | | | H20L15, H20L17, | SEQ ID NO: 28 | |
| | | | H20L6, H20L11 | | |
| HD46 | many | Heavy chain fusion | H22L13, H22L16, | U.S. Pat. No. | 5447 |
| | | protein | H22L18, H22L14, | 8,053,569 | |
| | | | H22L15, H22L17, | SEQ ID NO: 34 | |
| | | | H22L6, H22L11 | | |
| HD47 | many - growth | Heavy chain fusion | H5L11, H6L11, | U.S. Pat. No. | 5448 |
| | factors | protein | H14L11, H15L11, | 8,053,569 | |
| | | | H16L11, H17L11, | SEQ ID NO: 24 | |
| | | | H18L11, H19L11, | | |
| | | | H20L11, H21L11, | | |
| | | | H22L11, H23L11, | | |
| | | | H24L11, H25L11, | | |
| | | | H700L11 | | |
| HD48 | NOGO | Heavy chain | 2A10 construct | WO2007003421 | 5449 |
| | | humanized | | SEQ ID NO: 79 | |
| | | construct H1 | | | |
| HD49 | NOGO | Heavy chain | 2A10 construct | WO2007003421 | 5450 |
| | | humanized | | SEQ ID NO: 29 | |
| | | construct H14 | | | |
| HD50 | NOGO | Heavy chain | 2A10 construct | WO2007003421 | 5451 |
| | | humanized | | SEQ ID NO: 30 | |
| | | construct H15 | | | |
| HD51 | NOGO | Heavy chain | 2A10 construct | WO2007003421 | 5452 |
| | | humanized | | SEQ ID NO: 31 | |
| | | construct H16 | | | |
| HD52 | NOGO | Heavy chain | 2A10 construct | WO2007003421 | 5453 |
| | | humanized | | SEQ ID NO: 32 | |
| | | construct H17 | | | |
| HD53 | NOGO | Heavy chain | 2A10 construct | WO2007003421 | 5454 |
| | | humanized | | SEQ ID NO: 33 | |
| | | construct H18 | | | |
| HD54 | NOGO | Heavy chain | 2A10 construct | WO2007003421 | 5455 |
| | | humanized | | SEQ ID NO: 92 | |
| | | construct H19 | | | |
| HD55 | NOGO | Heavy chain | 2A10 construct | WO2007003421 | 5456 |
| | | humanized | | SEQ ID NO: 93 | |
| | | construct H20 | | | |
| HD56 | NOGO | Heavy chain | 2A10 construct | WO2007003421 | 5457 |
| | | humanized | | SEQ ID NO: 94 | |
| | | construct H21 | | | |
| HD57 | NOGO | Heavy chain | 2A10 construct | WO2007003421 | 5458 |
| | | humanized | | SEQ ID NO: 95 | |
| | | construct H22 | | | |
| HD58 | NOGO | Heavy chain | 2A10 construct | WO2007003421 | 5459 |
| | | humanized | | SEQ ID NO: 96 | |
| | | construct H23 | | | |
| HD59 | NOGO | Heavy chain | 2A10 construct | WO2007003421 | 5460 |
| | | humanized | | SEQ ID NO: 97 | |
| | | construct H24 | | | |
| HD60 | NOGO | Heavy chain | 2A10 construct | WO2007003421 | 5461 |
| | | humanized | | SEQ ID NO: 98 | |
| | | construct H25 | | | |
| HD61 | NOGO | Heavy chain | 2A10 construct | WO2007003421 | 5462 |
| | | humanized | | SEQ ID NO: 26 | |
| | | construct H5 | | | |
| HD62 | NOGO | Heavy chain | 2A10 construct | WO2007003421 | 5463 |
| | | humanized | | SEQ ID NO: 27 | |
| | | construct H6 | | | |
| HD63 | NOGO | Heavy chain | 2A10 construct | WO2007003421 | 5464 |
| | | humanized | | SEQ ID NO: 28 | |
| | | construct H700 | | | |
| HD64 | RTN4 (NOGO) | Heavy chain IgG4, | Atinumab | U.S. Pat. No. | 5465 |
| | | immunomodulator | | 8,163,285 | |
| | | | | SEQ ID NO: 24 | |
| HD65 | amyloid | Heavy chain | F11G3 | U.S. Pat. No. | 5466 |
| | oligomers | variable region | | 9,125,846 | |
| | | | | SEQ ID NO: 11 | |
| HD66 | DR6 and P75 | Heavy chain | 1P1D6.3 | WO2010062904 | 5467 |
| | | variable region | | SEQ ID NO: 107 | |
| HD67 | DR6 and P75 | Heavy chain | M50-H01 | WO2010062904 | 5468 |
| | | variable region | | SEQ ID NO: 7 | |
| HD68 | DR6 and P75 | Heavy chain | M67-G02 | WO2010062904 | 5469 |
| | | variable region | | SEQ ID NO: 77 | |
| HD69 | DR6 and P75 | Heavy chain | M72-F03 | WO2010062904 | 5470 |
| | | variable region | | SEQ ID NO: 87 | |
| HD70 | DR6 and P75 | Heavy chain | M73-C04 | WO2010062904 | 5471 |
| | | variable region | | SEQ ID NO: 97 | |
| HD71 | DR6 and P75 | Heavy chain | 1P2F2.1 | WO2010062904 | 5472 |
| | | variable region | | SEQ ID NO: 117 | |
| HD72 | DR6 and P75 | Heavy chain | 1P5D10.2 | WO2010062904 | 5473 |
| | | variable region | | SEQ ID NO: 127 | |
| HD73 | DR6 and P75 | Heavy chain | M51-H09 | WO2010062904 | 5474 |
| | | variable region | | SEQ ID NO: 17 | |
| HD74 | DR6 and P75 | Heavy chain | M53-E04 | WO2010062904 | 5475 |
| | | variable region | | SEQ ID NO: 27 | |
| HD75 | DR6 and P75 | Heavy chain | M53-F04 | WO2010062904 | 5476 |
| | | variable region | | SEQ ID NO: 37 | |
| HD76 | DR6 and P75 | Heavy chain | M62-B02 | WO2010062904 | 5477 |
| | | variable region | | SEQ ID NO: 47 | |
| HD77 | DR6 and P75 | Heavy chain | M63-E10 | WO2010062904 | 5478 |
| | | variable region | | SEQ ID NO: 57 | |
| HD78 | DR6 and P75 | Heavy chain | M66-B03 | WO2010062904 | 5479 |
| | | variable region | | SEQ ID NO: 67 | |
| HD79 | LPG | Heavy chain | #7 | U.S. Pat. No. | 5480 |
| | (lysophos- | variable region | | 8,591,902 | |
| | phatidyl- | | | SEQ ID NO: 18 | |
| | glucoside) | | | | |
| HD80 | LPG | Heavy chain | #15 | U.S. Pat. No. | 5481 |
| | (lysophos- | variable region | | 8,591,902 | |
| | phatidyl- | | | SEQ ID NO: 8 | |
| | glucoside) | | | | |
| HD81 | MAG | Heavy chain | | U.S. Pat. No. | 5482 |
| | | variable region | | 8,071,731 | |
| | | | | SEQ ID NO: 13 | |
| HD82 | MAG | Heavy chain | | U.S. Pat. No. | 5483 |
| | | variable region | | 8,071,731 | |
| | | | | SEQ ID NO: 14 | |
| HD83 | MAG | Heavy chain | | U.S. Pat. No. | 5484 |
| | | variable region | | 8,071,731 | |
| | | | | SEQ ID NO: 15 | |
| HD84 | MAI (myelin | Heavy chain | | WO2013158748 | 5485 |
| | associated | variable region | | SEQ ID NO: 1 | |
| | inhibitor) | | | | |
| HD85 | MAI (myelin | Heavy chain | | WO2013158748 | 5486 |
| | associated | variable region | | SEQ ID NO: 17 | |
| | inhibitor) | | | | |
| HD86 | NOGO | Heavy chain | H5L13, H5L16, | US20140147435 | 5487 |
| | | variable region | H5L18, H5L14, | SEQ ID NO: 11 | |
| | | | H5L15, H5L17, H5L6, | | |
| | | | H5L11 | | |
| HD87 | NOGO | Heavy chain | H6L 13, H6L16, | US20140147435 | 5488 |
| | | variable region | H6L18, H6L14, | SEQ ID NO: 12 | |
| | | | H6L15, H6L17, H6L6 | | |
| HD88 | NOGO | Heavy chain | H700L13, H700L 16, | US20140147435 | 5489 |
| | | variable region | H700L18, H700L 14, | SEQ ID NO: 13 | |
| | | | H700L15, H700L 17, | | |
| | | | H700L6, H700L11 | | |
| HD89 | NOGO | Heavy chain | H14L13, H14L16, | US20140147435 | 5490 |
| | | variable region | H14L18, H14L14, | SEQ ID NO: 14 | |
| | | | H14L15, H14L17, | | |
| | | | H14L6, H14L11 | | |
| HD90 | NOGO | Heavy chain | H15L13, H15L16, | US20140147435 | 5491 |
| | | variable region | H15L18, H15L14, | SEQ ID NO: 15 | |
| | | | H15L15, H15L17, | | |
| | | | H15L6, H15L11 | | |
| HD91 | NOGO | Heavy chain | H16L13, H16L16, | US20140147435 | 5492 |
| | | variable region | H16L18, H16L14, | SEQ ID NO: 16 | |
| | | | H16L15, H16L17, | | |
| | | | H16L6, H16L11 | | |
| HD92 | NOGO | Heavy chain | H17L13, H17L16, | US20140147435 | 5493 |
| | | variable region | H17L18, H17L14, | SEQ ID NO: 17 | |
| | | | H17L15, H17L17, | | |
| | | | H17L6, H17L11 | | |
| HD93 | NOGO | Heavy chain | H18L13, H18L16, | US20140147435 | 5494 |
| | | variable region | H18L18, H18L14, | SEQ ID NO: 18 | |
| | | | H18L15, H18L17, | | |
| | | | H18L6, H18L11 | | |
| HD94 | NOGO | Heavy chain | H1L13, H1L16, | US20140147435 | 5495 |
| | | variable region | H1L18, H1L14, | SEQ ID NO: 77 | |
| | | | H1L15, H1L17, H1L6 | | |
| HD95 | NOGO | Heavy chain | H19L13, H19L16, | US20140147435 | 5496 |
| | | variable region | H19L18, H19L14, | SEQ ID NO: 85 | |
| | | | H19L15, H19L17, | | |
| | | | H19L6. H19L11 | | |
| HD96 | NOGO | Heavy chain | H20L13, H20L16, | US20140147435 | 5497 |
| | | variable region | H20L18, H20L14, | SEQ ID NO: 86 | |
| | | | H20L15, H20L17, | | |
| | | | H20L6, H20L11 | | |
| HD97 | NOGO | Heavy chain | H21L13, H21L16, | US20140147435 | 5498 |
| | | variable region | H21L18, H21L14, | SEQ ID NO: 87 | |
| | | | H21L15, H21L17, | | |
| | | | H21L6, H21L11 | | |
| HD98 | NOGO | Heavy chain | H22L13, H22L16, | US20140147435 | 5499 |
| | | variable region | H22L18, H22L14, | SEQ ID NO: 88 | |
| | | | H22L15, H22L17, | | |
| | | | H22L6, H22L11 | | |
| HD99 | NOGO | Heavy chain | H23L13, H23L16, | US20140147435 | 5500 |
| | | variable region | H23L18, H23L14, | SEQ ID NO: 89 | |
| | | | H23L15, H23L17, | | |
| | | | H23L6. H23L11 | | |
| HD100 | NOGO | Heavy chain | H24L13, H24L16, | US20140147435 | 5501 |
| | | variable region | H24L18, H24L14, | SEQ ID NO: 90 | |
| | | | H24L15, H24L17, | | |
| | | | H24L6, H24L11 | | |
| HD101 | NOGO | Heavy chain | H25L13, H25L16, | US20140147435 | 5502 |
| | | variable region | H25L18, H25L14, | SEQ ID NO: 91 | |
| | | | H25L15, H25L17, | | |
| | | | H25L6, H25L11 | | |
| HD102 | Nogo-66 | Heavy chain | Antibody clone 50 | US20140065155 | 5503 |
| | | variable region | | SEQ ID NO: 3 | |
| HD103 | Nogo-66 | Heavy chain | Antibody clone 51 | US20140065155 | 5504 |
| | | variable region | | SEQ ID NO: 5 | |
| HD104 | NogoA/NIG | Heavy chain | 6A3-Ig4 | WO2009056509 | 5505 |
| | | variable region | | SEQ ID NO: 24 | |
| HD105 | NogoA/NiG | Heavy chain | 6A3-IgG1 | WO2009056509 | 5506 |
| | | variable region | | SEQ ID NO: 4 | |
| HD106 | RGM A | Heavy chain | 5F9.1-GL | US20150183871 | 5507 |
| | | variable region | | SEQ ID NO: 35 | |
| HD107 | RGM A | Heavy chain | 5F9.2-GL | US20150183871 | 5508 |
| | | variable region | | SEQ ID NO: 36 | |
| HD108 | RGM A | Heavy chain | 5F9.3-GL | US20150183871 | 5509 |
| | | variable region | | SEQ ID NO: 37 | |
| HD109 | RGM A | Heavy chain | 5F9.4-GL | US20150183871 | 5510 |
| | | variable region | | SEQ ID NO: 38 | |
| HD110 | RGM A | Heavy chain | 5F9.5-GL | US20150183871 | 5511 |
| | | variable region | | SEQ ID NO: 39 | |
| HD111 | RGM A | Heavy chain | 5F9.6-GL | US20150183871 | 5512 |
| | | variable region | | SEQ ID NO: 40 | |
| HD112 | RGM A | Heavy chain | 5F9.7-GL | US20150183871 | 5513 |
| | | variable region | | SEQ ID NO: 41 | |
| HD113 | RGM A | Heavy chain | 5F9.8-GL | US20150183871 | 5514 |
| | | variable region | | SEQ ID NO: 42 | |
| HD114 | RGM A | Heavy chain | 5F9.9-GL | US20150183871 | 5515 |
| | | variable region | | SEQ ID NO: 43 | |
| HD115 | RGM A | Heavy chain | hSF9.1, h5F9.1, | US2015/0183871 | 5516 |
| | | variable region | h5F9.1, h5F9.1, | SEQ ID NO: 47 | |
| | | | h5F9.1, h5F9.2, | | |
| | | | h5F9.3 | | |
| HD116 | RGM A | Heavy chain | h5F9.3, h5F9.9, | US2015/0183871 | 5517 |
| | | variable region | h5F9.25 | SEQ ID NO: 53 | |
| HD117 | RGM A | Heavy chain | h5F9.4, h5F9.10, | US2015/0183871 | 5518 |
| | | variable region | h5F9.26 | SEQ ID NO: 54 | |
| HD118 | RGMa | Heavy chain | AE12-21 | US20140023659 | 5519 |
| | | variable region | | SEQ ID NO: 115 | |
| HD119 | RGMa | Heavy chain | AE12-23 | US20140023659 | 5520 |
| | | variable region | | SEQ ID NO: 123 | |
| HD120 | RGMa | Heavy chain | AE12-24 | US20140023659 | 5521 |
| | | variable region | | SEQ ID NO: 131 | |
| HD121 | RGMa | Heavy chain | AE12-3 | US20140023659 | 5522 |
| | | variable region | | SEQ ID NO: 17 | |
| HD122 | RGMa | Heavy chain | AE12-4 | US20140023659 | 5523 |
| | | variable region | | SEQ ID NO: 25 | |
| HD123 | RGMa | Heavy chain | AE12-5 | US20140023659 | 5524 |
| | | variable region | | SEQ ID NO: 33 | |
| HD124 | RGMa | Heavy chain | AE12-6 | US20140023659 | 5525 |
| | | variable region | | SEQ ID NO: 41 | |
| HD125 | RGMa | Heavy chain | AE12-7 | US20140023659 | 5526 |
| | | variable region | | SEQ ID NO: 49 | |
| HD126 | RGMa | Heavy chain | AE12-8 | US20140023659 | 5527 |
| | | variable region | | SEQ ID NO: 57 | |
| HD127 | RGMa | Heavy chain | AE12-2 | US20140023659 | 5528 |
| | | variable region | | SEQ ID NO: 9 | |
| HD128 | RGMa | Heavy chain | AE12-13 | US20140023659 | 5529 |
| | | variable region | | SEQ ID NO: 91 | |
| HD129 | RGMa | Heavy chain | AE12-15 | US20140023659 | 5530 |
| | | variable region | | SEQ ID NO: 99 | |
| HD130 | RGMa | Heavy chain | AE12-1 | US20140023659 | 5531 |
| | | variable region | | SEQ ID NO: 1 | |
| HD131 | RGMa | Heavy chain | AE12-20 | US20140023659 | 5532 |
| | | variable region | | SEQ ID NO: 107 | |
| HD132 | NOGO | Heavy chain | 2A10 construct | WO2007003421 | 5533 |
| | | variable region | | SEQ ID NO: 77 | |
| | | humanized | | | |
| | | construct H1 | | | |
| HD133 | NOGO | Heavy chain | 2A10 construct | WO2007003421 | 5534 |
| | | variable region | | SEQ ID NO: 14 | |
| | | humanized | | | |
| | | construct H14 | | | |
| HD134 | NOGO | Heavy chain | 2A10 construct | WO2007003421 | 5535 |
| | | variable region | | SEQ ID NO: 15 | |
| | | humanized | | | |
| | | construct H15 | | | |
| HD135 | NOGO | Heavy chain | 2A10 construct | WO2007003421 | 5536 |
| | | variable region | | SEQ ID NO: 16 | |
| | | humanized | | | |
| | | construct H16 | | | |
| HD136 | NOGO | Heavy chain | 2A10 construct | WO2007003421 | 5537 |
| | | variable region | | SEQ ID NO: 17 | |
| | | humanized | | | |
| | | construct H17 | | | |
| HD137 | NOGO | Heavy chain | 2A10 construct | WO2007003421 | 5538 |
| | | variable region | | SEQ ID NO: 18 | |
| | | humanized | | | |
| | | construct H18 | | | |
| HD138 | NOGO | Heavy chain | 2A10 construct | WO2007003421 | 5539 |
| | | variable region | | SEQ ID NO: 85 | |
| | | humanized | | | |
| | | construct H19 | | | |
| HD139 | NOGO | Heavy chain | 2A10 construct | WO2007003421 | 5540 |
| | | variable region | | SEQ ID NO: 86 | |
| | | humanized | | | |
| | | construct H20 | | | |
| HD140 | NOGO | Heavy chain | 2A10 construct | WO2007003421 | 5541 |
| | | variable region | | SEQ ID NO: 87 | |
| | | humanized | | | |
| | | construct H21 | | | |
| HD141 | NOGO | Heavy chain | 2A10 construct | WO2007003421 | 5542 |
| | | variable region | | SEQ ID NO: 88 | |
| | | humanized | | | |
| | | construct H22 | | | |
| HD142 | NOGO | Heavy chain | 2A10 construct | WO2007003421 | 5543 |
| | | variable region | | SEQ ID NO: 89 | |
| | | humanized | | | |
| | | construct H23 | | | |
| HD143 | NOGO | Heavy chain | 2A10 construct | WO2007003421 | 5544 |
| | | variable region | | SEQ ID NO: 90 | |
| | | humanized | | | |
| | | construct H24 | | | |
| HD144 | NOGO | Heavy chain | 2A10 construct | WO2007003421 | 5545 |
| | | variable region | | SEQ ID NO: 91 | |
| | | humanized | | | |
| | | construct H25 | | | |
| HD145 | NOGO | Heavy chain | 2A10 construct | WO2007003421 | 5546 |
| | | variable region | | SEQ ID NO: 11 | |
| | | humanized | | | |
| | | construct H5 | | | |
| HD146 | NOGO | Heavy chain | 2A10 construct | WO2007003421 | 5547 |
| | | variable region | | SEQ ID NO: 12 | |
| | | humanized | | | |
| | | construct H6 | | | |
| HD147 | NOGO | Heavy chain | 2A10 construct | WO2007003421 | 5548 |
| | | variable region | | SEQ ID NO: 13 | |
| | | humanized | | | |
| | | construct H700 | | | |
| HD 148 | amy loids | Light chain | #118 | WO2010012004 | 5549 |
| | | | | SEQ ID NO: 10 | |
| HD 149 | amyloids | Light chain | #121 | WO2010012004 | 5550 |
| | | | | SEQ ID NO: 12 | |
| HD 150 | amyloids | Light chain | #201 | WO2010012004 | 5551 |
| | | | | SEQ ID NO: 14 | |
| HD151 | amyloids | Light chain | #204 | WO2010012004 | 5552 |
| | | | | SEQ ID NO: 15 | |
| HD 152 | amyloids | Light chain | #205 | WO2010012004 | 5553 |
| | | | | SEQ ID NO: 17 | |
| HD153 | NOGO | Light chain | H6L13 FL, H19L13 | US20140147435 | 5554 |
| | | | FL, H20L13 FL, | SEQ ID NO: 35 | |
| | | | H21L13 FL, H25L13 | | |
| | | | FL | | |
| HD154 | NOGO | Light chain | H16L16 FL, H19L16 | US20140147435 | 5555 |
| | | | FL, H20L16 FL, | SEQ ID NO: 38 | |
| | | | H21L16 FL, H25L16 | | |
| | | | FL, H18L16 FL | | |
| HD155 | NOGO | Light chain | H16L18 FL, H19L18 | US20140147435 | 5556 |
| | | | FL, H20L18 FL, | SEQ ID NO: 40 | |
| | | | H21L18 FL, H25L18 | | |
| | | | FL | | |
| HD156 | Nogo receptor- | Light chain | 7E11 | US20090215691 | 5557 |
| | 1 | | | SEQ ID NO: 15 | |
| HD157 | Nogo receptor- | Light chain | 7E11 | US20090215691 | 5558 |
| | 1 | | | SEQ ID NO: 17 | |
| HD158 | trk-C (NT-3 | Light chain | 2250 | U.S. Pat. No. | 5559 |
| | trkC ligand) | | | 7,615,383 | |
| | | | | SEQ ID NO: 49 | |
| HD159 | trk-C (NT-3 | Light chain | 2253 | U.S. Pat. No. | 5560 |
| | trkC ligand) | | | 7,615,383 | |
| | | | | SEQ ID NO: 50 | |
| HD160 | trk-C (NT-3 | Light chain | 2256 | U.S. Pat. No. | 5561 |
| | trkC ligand) | | | 7,615,383 | |
| | | | | SEQ ID NO: 51 | |
| HD161 | trk-C (NT-3 | Light chain | 6.1.2 | U.S. Pat. No. | 5562 |
| | trkC ligand) | | | 7,615,383 | |
| | | | | SEQ ID NO: 52 | |
| HD162 | trk-C (NT-3 | Light chain | 6.4.1 | U.S. Pat. No. | 5563 |
| | trkC ligand) | | | 7,615,383 | |
| | | | | SEQ ID NO: 53 | |
| HD163 | trk-C (NT-3 | Light chain | 2345 | U.S. Pat. No. | 5564 |
| | trkC ligand) | | | 7,615,383 | |
| | | | | SEQ ID NO: 54 | |
| HD164 | trk-C (NT-3 | Light chain | 2349 | U.S. Pat. No. | 5565 |
| | trkC ligand) | | | 7,615,383 | |
| | | | | SEQ ID NO: 55 | |
| HD165 | many | Light chain fusion | H21L13, H21L16, | U.S. Pat. No. | 5566 |
| | | protein | H21L18, H21L14, | 8,053,569 | |
| | | | H21L15, H21L17, | SEQ ID NO: 31 | |
| | | | H21L6, H21L11 | | |
| HD166 | many | Light chain fusion | H23L13, H23L16, | U.S. Pat. No. | 5567 |
| | | protein | H23L18, H23L14, | 8,053,569 | |
| | | | H23L15, H23L17, | SEQ ID NO: 36 | |
| | | | H23L6, H23L11 | | |
| HD167 | NOGO | Light chain | 2A10 construct | WO2007003421 | 5568 |
| | | humanized | | SEQ ID NO: 80 | |
| | | construct L11 | | | |
| HD168 | NOGO | Light chain | 2A10 construct | WO2007003421 | 5569 |
| | | humanized | | SEQ ID NO: 35 | |
| | | construct L13 | | | |
| HD169 | NOGO | Light chain | 2A10 construct | WO2007003421 | 5570 |
| | | humanized | | SEQ ID NO: 36 | |
| | | construct L 14 | | | |
| HD170 | NOGO | Light chain | 2A10 construct | WO2007003421 | 5571 |
| | | humanized | | SEQ ID NO: 37 | |
| | | construct L15 | | | |
| HD171 | NOGO | Light chain | 2A10 construct | WO2007003421 | 5572 |
| | | humanized | | SEQ ID NO: 38 | |
| | | construct L16 | | | |
| HD172 | NOGO | Light chain | 2A10 construct | WO2007003421 | 5573 |
| | | humanized | | SEQ ID NO: 39 | |
| | | construct L17 | | | |
| HD173 | NOGO | Light chain | 2A10 construct | WO2007003421 | 5574 |
| | | humanized | | SEQ ID NO: 40 | |
| | | construct L18 | | | |
| HD174 | NOGO | Light chain | 2A10 construct | WO2007003421 | 5575 |
| | | humanized | | SEQ ID NO: 34 | |
| | | construct L6 | | | |
| HD175 | RTN4 | Light chain IgG4, | Atinumab | U.S. Pat. No. | 5576 |
| | | immunomodulator | | 8,163,285 | |
| | | | | SEQ ID NO: 25 | |
| HD176 | huntingtin | Light chain single | | US20050226863 | 5577 |
| | protein | domain | | SEQ ID NO: 1 | |
| HD177 | huntingtin | Light chain single | VL12.3 | US20050226863 | 5578 |
| | protein | domain | | SEQ ID NO: 10 | |
| HD178 | huntingtin | Light chain single | | US20050226863 | 5579 |
| | protein | domain | | SEQ ID NO: 2 | |
| HD179 | huntingtin | Light chain single | | US20050226863 | 5580 |
| | protein | domain | | SEQ ID NO: 3 | |
| HD180 | huntingtin | Light chain single | | US20050226863 | 5581 |
| | protein | domain | | SEQ ID NO: 4 | |
| HD181 | amyloid | Light chain | F11G3 | U.S. Pat. No. | 5582 |
| | oligomers | variable region | | 9,125,846 | |
| | | | | SEQ ID NO: 12 | |
| HD182 | DR6 and P75 | Light chain | M73-C04 | WO2010062904 | 5583 |
| | | variable region | | SEQ ID NO: 102 | |
| HD183 | DR6 and P75 | Light chain | IP1D6.3 | WO2010062904 | 5584 |
| | | variable region | | SEQ ID NO: 112 | |
| HD184 | DR6 and P75 | Light chain | M50-H02 | WO2010062904 | 5585 |
| | | variable region | | SEQ ID NO: 12 | |
| HD185 | DR6 and P75 | Light chain | 1P2F2.1 | WO2010062904 | 5586 |
| | | variable region | | SEQ ID NO: 122 | |
| HD186 | DR6 and P75 | Light chain | 1P5D10.2 | WO2010062904 | 5587 |
| | | variable region | | SEQ ID NO: 132 | |
| HD187 | DR6 and P75 | Light chain | M51-H09 | WO2010062904 | 5588 |
| | | variable region | | SEQ ID NO: 22 | |
| HD188 | DR6 and P75 | Light chain | M53-E04 | WO2010062904 | 5589 |
| | | variable region | | SEQ ID NO: 32 | |
| HD189 | DR6 and P75 | Light chain | M53-F04 | WO2010062904 | 5590 |
| | | variable region | | SEQ ID NO: 42 | |
| HD190 | DR6 and P75 | Light chain | M62-B02 | WO2010062904 | 5591 |
| | | variable region | | SEQ ID NO: 52 | |
| HD191 | DR6 and P75 | Light chain | M63-E10 | WO2010062904 | 5592 |
| | | variable region | | SEQ ID NO: 62 | |
| HD192 | DR6 and P75 | Light chain | M66-B03 | WO2010062904 | 5593 |
| | | variable region | | SEQ ID NO: 72 | |
| HD193 | DR6 and P75 | Light chain | M67-G02 | WO2010062904 | 5594 |
| | | variable region | | SEQ ID NO: 82 | |
| HD194 | DR6 and P75 | Light chain | M72-F03 | WO2010062904 | 5595 |
| | | variable region | | SEQ ID NO: 92 | |
| HD195 | LPG | Light chain | #7 | U.S. Pat. No. | 5596 |
| | (lysophos- | variable region | | 8,591,902 | |
| | phatidyl- | | | SEQ ID NO: 17 | |
| | glucoside) | | | | |
| HD196 | LPG | Light chain | #15 | U.S. Pat. No. | 5597 |
| | (lysophos- | variable region | | 8,591,902 | |
| | phatidyl- | | | SEQ ID NO: 7 | |
| | glucoside) | | | | |
| HD197 | MAG | Light chain | | U.S. Pat. No. | 5598 |
| | | variable region | | 8,071,731 | |
| | | | | SEQ ID NO: 16 | |
| HD198 | MAG | Light chain | | U.S. Pat. No. | 5599 |
| | | variable region | | 8,071,731 | |
| | | | | SEQ ID NO: 17 | |
| HD199 | MAG | Light chain | | U.S. Pat. No. | 5600 |
| | | variable region | | 8,071,731 | |
| | | | | SEQ ID NO: 18 | |
| HD200 | MAG | Light chain | | U.S. Pat. No. | 5601 |
| | | variable region | | 8,071,731 | |
| | | | | SEQ ID NO: 19 | |
| HD201 | MAI (myelin | Light chain | | WO2013158748 | 5602 |
| | associated | variable region | | SEQ ID NO: 11 | |
| | inhibitor) | | | | |
| HD202 | MAI (myelin | Light chain | | WO2013158748 | 5603 |
| | associated | variable region | | SEQ ID NO: 27 | |
| | inhibitor) | | | | |
| HD203 | NOGO | Light chain | H1L6, H5L6, H6L6, | US20140147435 | 5604 |
| | | variable region | H14L6, H15L6, | SEQ ID NO: 19 | |
| | | | H16L6, H17L6, | | |
| | | | H18L6, H19L6, | | |
| | | | H20L6, H21L6, | | |
| | | | H22L6, H23L6, | | |
| | | | H24L6, H25L6, | | |
| | | | H700L6 | | |
| HD204 | NOGO | Light chain | H1L13, H5L13, | US20140147435 | 5605 |
| | | variable region | H6L13, H14L13, | SEQ ID NO: 20 | |
| | | | H15L13, H16L13, | | |
| | | | H17L13, H18L13, | | |
| | | | H19L13, H20L13, | | |
| | | | H21L13, H22L13, | | |
| | | | H23L13, H24L13 | | |
| | | | H25L13, H700L13 | | |
| HD205 | NOGO | Light chain | H1L14, H5L14, | US20140147435 | 5606 |
| | | variable region | H6L14, H14L14, | SEQ ID NO: 21 | |
| | | | H15L14, H16L14, | | |
| | | | H17L14, H18L14, | | |
| | | | H19L14, H20L14, | | |
| | | | H21L14, H22L14, | | |
| | | | H23L14, H24L14, | | |
| | | | H25L14, H700L14 | | |
| HD206 | NOGO | Light chain | H1L15, H5L15, | US20140147435 | 5607 |
| | | variable region | H6L15, H14L15, | SEQ ID NO: 22 | |
| | | | H15L15, H16L15, | | |
| | | | H17L15, H18L15, | | |
| | | | H19L15, H20L15, | | |
| | | | H21L15, H22L15, | | |
| | | | H23L15, H24L15, | | |
| | | | H25L15, H700L15 | | |
| HD207 | NOGO | Light chain | H1L 16, H5L 16, | US20140147435 | 5608 |
| | | variable region | H6L 16, H14L16, | SEQ ID NO: 23 | |
| | | | H15L16, H16L16, | | |
| | | | H17L16, H18L16, | | |
| | | | H19L16, H20L16, | | |
| | | | H21L16, H22L16, | | |
| | | | H23L16, H24L16, | | |
| | | | H25L16, H700L16 | | |
| HD208 | NOGO | Light chain | H1L17, H5L17, | US20140147435 | 5609 |
| | | variable region | H6L17, H14L17, | SEQ ID NO: 24 | |
| | | | H15L17, H16L17, | | |
| | | | H17L17, H18L17, | | |
| | | | H19L17, H20L17, | | |
| | | | H21L17, H22L17, | | |
| | | | H23L17, H24L17, | | |
| | | | H25L17, H700L17 | | |
| HD209 | NOGO | Light chain | H1L18, H5L18, | US20140147435 | 5610 |
| | | variable region | H6L18, H14L18, | SEQ ID NO: 25 | |
| | | | H15L18, H16L18, | | |
| | | | H17L18, H18L18, | | |
| | | | H19L18, H20L18, | | |
| | | | H21L18, H22L18, | | |
| | | | H23L18, H24L18, | | |
| | | | H25L18, H700L18 | | |
| HD210 | NOGO | Light chain | H5L11, H6L11, | US20140147435 | 5611 |
| | | variable region | H14L11, H15L11, | SEQ ID NO: 78 | |
| | | | H16L11, H17L11, | | |
| | | | H18L11, H19L11 | | |
| | | | H20L11, H21L11, | | |
| | | | H22L11, H23L11, | | |
| | | | H24L11, H25L11, | | |
| | | | H700L11 | | |
| HD211 | Nogo-66 | Light chain | Antibody clone 50 | US20140065155 | 5612 |
| | | variable region | | SEQ ID NO: 4 | |
| HD212 | Nogo-66 | Light chain | Antibody clone 51 | US20140065155 | 5613 |
| | | variable region | | SEQ ID NO: 6 | |
| HD213 | NogoA/NiG | Light chain | 6A3-Ig4 | WO2009056509 | 5614 |
| | | variable region | | SEQ ID NO: 25 | |
| HD214 | NogoA/NIG | Light chain | 6A3-IgG1 | WO2009056509 | 5615 |
| | | variable region | | SEQ ID NO: 5 | |
| HD215 | RGM A | Light chain | 5F9.1-GL, 5F9.1-GL, | US20150183871 | 5616 |
| | | variable region | 5F9.1-GL, 5F9.1-GL, | SEQ ID NO: 44 | |
| | | | 5F9.1-GL, 5F9.1-GL, | | |
| | | | 5F9.1-GL, 5F9.1-GL, | | |
| | | | 5F9.1-GL, 5F9.1-GL, | | |
| | | | h5F9.4, h5F9.11, | | |
| | | | h5F9.12 | | |
| HD216 | RGM A | Light chain | 5F9.2-GL, 5F9.2-GL, | US20150183871 | 5617 |
| | | variable region | 5F9.2-GL, 5F9.2-GL, | SEQ ID NO: 45 | |
| | | | 5F9.2-GL, 5F9.2-GL, | | |
| | | | 5F9.2-GL, 5F9.2-GL, | | |
| | | | 5F9.2-GL, 5F9.2-GL, | | |
| | | | h5F9.5, h5F9.19, | | |
| | | | h5F9.20 | | |
| HD217 | RGM A | Light chain | 5F9.3-GL, 5F9.3-GL, | US20150183871 | 5618 |
| | | variable region | 5F9.3~GL, 5F9.3-GL, | SEQ ID NO: 46 | |
| | | | 5F9.3-GL, 5F9.3-GL, | | |
| | | | 5F9.3-GL, 5F9.3-GL, | | |
| | | | 5F9.3-GL, 5F9.3-GL, | | |
| | | | h5F9.6, h5F9.21, | | |
| | | | h5F9.22 | | |
| HD218 | RGM A | Light chain | h5F9.5, 15F9.6, | US20150183871 | 5619 |
| | | variable region | h5F9.7, h5F9.8, | SEQ ID NO: 48 | |
| | | | h5F9.9, h5F9.10 | | |
| HD219 | RGM A | Light chain | h5F9.11, | US20150183871 | 5620 |
| | | variable region | h5F9.19, h5F9.21 | SEQ ID NO: 49 | |
| HD220 | RGM A | Light chain | h5F9.12, h5F9.20, | US20150183871 | 5621 |
| | | variable region | h5F9.22, h5F9.23, | SEQ ID NO: 50 | |
| | | | h5F9.25, h5F9.25, | | |
| | | | h5F9.26 | | |
| HD221 | RGM A | Light chain | h5F9.1, h5F9.7, | US20150183871 | 5622 |
| | | variable region | h5F9.23 | SEQ ID NO: 51 | |
| HD222 | RGM A | Light chain | h5F9.2, h5F9.8, | US20150183871 | 5623 |
| | | variable region | h5F9.25 | SEQ ID NO: 52 | |
| HD223 | RGMa | Light chain | AE12-15 | US20140023659 | 5624 |
| | | variable region | | SEQ ID NO: 103 | |
| HD224 | RGMa | Light chain | AE12-20 | US20140023659 | 5625 |
| | | variable region | | SEQ ID NO: 111 | |
| HD225 | RGMa | Light chain | AE12-21 | US20140023659 | 5626 |
| | | variable region | | SEQ ID NO: 119 | |
| HD226 | RGMa | Light chain | AE12-23 | US20140023659 | 5627 |
| | | variable region | | SEQ ID NO: 127 | |
| HD227 | RGMa | Light chain | AE12-2 | US20140023659 | 5628 |
| | | variable region | | SEQ ID NO: 13 | |
| HD228 | RGM | Light chain | AE12-24 | US20140023659 | 5629 |
| | | variable region | | SEQ ID NO: 135 | |
| HD229 | RGMa | Light chain | AE12-3 | US20140023659 | 5630 |
| | | variable region | | SEQ ID NO: 21 | |
| HD230 | RGMa | Light chain | AE12-4 | US20140023659 | 5631 |
| | | variable region | | SEQ ID NO: 29 | |
| HD231 | RGMa | Light chain | AE12-5 | US20140023659 | 5632 |
| | | variable region | | SEQ ID NO: 37 | |
| HD232 | RGMa | Light chain | AE12-6 | US20140023659 | 5633 |
| | | variable region | | SEQ ID NO: 45 | |
| HD233 | RGMa | Light chain | AE12-1 | US20140023659 | 5634 |
| | | variable region | | SEQ ID NO: 5 | |
| HD234 | RGMa | Light chain | AE12-7 | US20140023659 | 5635 |
| | | variable region | | SEQ ID NO: 53 | |
| HD235 | RGMa | Light chain | AE12-8 | US20140023659 | 5636 |
| | | variable region | | SEQ ID NO: 61 | |
| HD236 | RGMa | Light chain | AE12-13 | US20140023659 | 5637 |
| | | variable region | | SEQ ID NO: 95 | |
| HD237 | NOGO | Light chain | 2A10 construct | WO2007003421 | 5638 |
| | | variable region | | SEQ ID NO: 78 | |
| | | humanized | | | |
| | | construct L11 | | | |
| HD238 | NOGO | Light chain | 2A10 construct | WO2007003421 | 5639 |
| | | variable region | | SEQ ID NO: 20 | |
| | | humanized | | | |
| | | construct L13 | | | |
| HD239 | NOGO | Light chain | 2A10 construct | WO2007003421 | 5640 |
| | | variable region | | SEQ ID NO: 21 | |
| | | humanized | | | |
| | | construct L14 | | | |
| HD240 | NOGO | Light chain | 2A10 construct | WO2007003421 | 5641 |
| | | variable region | | SEQ ID NO: 22 | |
| | | humanized | | | |
| | | construct L15 | | | |
| HD241 | NOGO | Light chain | 2A10 construct | WO2007003421 | 5642 |
| | | variable region | | SEQ ID NO: 23 | |
| | | humanized | | | |
| | | construct L16 | | | |
| HD242 | NOGO | Light chain | 2A10 construct | WO2007003421 | 5643 |
| | | variable region | | SEQ ID NO: 24 | |
| | | humanized | | | |
| | | construct L 17 | | | |
| HD243 | NOGO | Light chain | 2A10 construct | WO2007003421 | 5644 |
| | | variable region | | SEQ ID NO: 25 | |
| | | humanized | | | |
| | | construct L18 | | | |
| HD244 | NOGO | Light chain | 2A10 construct | WO2007003421 | 5645 |
| | | variable region | | SEQ ID NO: 19 | |
| | | humanized | | | |
| | | construct L6 | | | |
| HD245 | HTT | | | Lecerf, J.M. et al., | 5646 |
| | | | | Human single- | |
| | | | | chain Fv | |
| | | | | intrabodies | |
| | | | | counteract in situ | |
| | | | | huntingtin | |
| | | | | aggregation in | |
| | | | | cellular models of | |
| | | | | Huntington's | |
| | | | | disease, Proc. Natl. | |
| | | | | Acad. Sci. U.S.A. | |
| | | | | 98 (8). 4764-4769 | |
| | | | | (2001), NCBI | |
| | | | | Accession # | |
| | | | | ACA53373.1 | |

### Muscle Disease Antibodies

In some embodiments, the payload region of the viral particle comprises one or more nucleic acid sequences encoding one or more of the muscle disease payload antibody polypeptides listed in Table 6 (MUS1-MUS485; SEQ ID NO: 5647-6131). A non-exhaustive listing of muscle diseases includes Multiple System Atrophy (MSA), Amyotrophic Lateral Sclerosis (ALS) and Duchenne Muscular Dystrophy (DMD).

TABLE 6
Muscle Disease Antibodies

| Antibody | | | | Reference | SEQ ID |
|---|---|---|---|---|---|
| No. | Target | Description | Antibody Name | Information | NO |
| MUS1 | amyloid | consensus sequence | M13 g3p, fd g3p, f1 | US20150376239 | 5647 |
| | proteins | | g3p | SEQ ID NO: 4 | |
| MUS2 | amyloid | consensus sequence | 12-2 g3p, Ike g3p | US20150376239 | 5648 |
| | proteins | | | SEQ ID NO: 7 | |
| MUS3 | 118-126 of α- | constant region | IgG1 | US20150259404 | 5649 |
| | synuclein | | | SEQ ID NO: 38 | |
| MUS4 | amyloid | Fusion protein | M13 g3p | US20150376239 | 5650 |
| | proteins | | | SEQ ID NO: 1 | |
| MUS5 | amyloid | Fusion protein | Construct 5 | US20150376239 | 5651 |
| | proteins | | | SEQ ID NO: 11 | |
| MUS6 | amyloid | Fusion protein | Construct 6 | US20150376239 | 5652 |
| | proteins | | | SEQ ID NO: 13 | |
| MUS7 | amyloid | Fusion protein | fd N2 | US20150376239 | 5653 |
| | proteins | | | SEQ ID NO: 14 | |
| MUS8 | amyloid | Fusion protein | f1 N2 | US20150376239 | 5654 |
| | proteins | | | SEQ ID NO: 15 | |
| MUS9 | amyloid | Fusion protein | M13 N2 | US20150376239 | 5655 |
| | proteins | | | SEQ ID NO: 16 | |
| MUS10 | amyloid | Fusion protein | Ike N2 | US20150376239 | 5656 |
| | proteins | | | SEQ ID NO: 17 | |
| MUS11 | amyloid | Fusion protein | 12-2 N2 | US20150376239 | 5657 |
| | proteins | | | SEQ ID NO: 18 | |
| MUS12 | amyloid | Fusion protein | If1 N2 | US20150376239 | 5658 |
| | proteins | | | SEQ ID NO: 19 | |
| MUS13 | amyloid | Fusion protein | fd g3p | US20150376239 | 5659 |
| | proteins | | | SEQ ID NO: 2 | |
| MUS14 | amyloid | Fusion protein | Construct 3 | US20150376239 | 5660 |
| | proteins | | | SEQ ID NO: 20 | |
| MUS15 | amyloid | Fusion protein | Construct 3m g3p | US20150376239 | 5661 |
| | proteins | | portion | SEQ ID NO: 24 | |
| MUS16 | amyloid | Fusion protein | If1 g3p | US20150376239 | 5662 |
| | proteins | | | SEQ ID NO: 29 | |
| MUS17 | amyloid | Fusion protein | f1 g3p | US20150376239 | 5663 |
| | proteins | | | SEQ ID NO: 3 | |
| MUS18 | amyloid | Fusion protein | fd g3p | US20150376239 | 5664 |
| | proteins | | | SEQ ID NO: 30 | |
| MUS19 | amyloid | Fusion protein | Construct 8, rs-g3p | US20150376239 | 5665 |
| | proteins | | (If1-NIN2)-hlgG1-Fc | SEQ ID NO: 31 | |
| MUS20 | amyloid | Fusion protein | 12-2 g3p | US20150376239 | 5666 |
| | proteins | | | SEQ ID NO: 5 | |
| MUS21 | amyloid | Fusion protein | Ike g3p | US20150376239 | 5667 |
| | proteins | | | SEQ ID NO: 6 | |
| MUS22 | amyloid | Fusion protein | If1 g3p | US20150376239 | 5668 |
| | proteins | | | SEQ ID NO: 8 | |
| MUS23 | amyloid | Fusion protein | Construct 4 | US20150376239 | 5669 |
| | proteins | | | SEQ ID NO: 9 | |
| MUS24 | ACVR2B | Heavy chain | H6L4. H6L5, H6L6 | U.S. Pat. No. | 5670 |
| | (SMA - muscle | | | 8,388,968 | |
| | growth) | | | SEQ ID NO: 146 | |
| MUS25 | ACVR2B | Heavy chain | | U.S. Pat. No. | 5671 |
| | (SMA - muscle | | | 8,388,968 | |
| | growth) | | | SEQ ID NO: 146 | |
| MUS26 | amyloids | Heavy chain | #118 | WO2010012004 | 5672 |
| | | | | SEQ ID NO: 11 | |
| MUS27 | amyloids | Heavy chain | #121 | WO2010012004 | 5673 |
| | | | | SEQ ID NO: 13 | |
| MUS28 | amyloids | Heavy chain | #204 | WO2010012004 | 5674 |
| | | | | SEQ ID NO: 16 | |
| MUS29 | amyloids | Heavy chain | #205 | WO2010012004 | 5675 |
| | | | | SEQ ID NO: 18 | |
| MUS30 | EAG1 | Heavy chain | chimeric ImAb3 | WO2006037604 | 5676 |
| | | | | SEQ ID NO: 12 | |
| MUS31 | EAG1 | Heavy chain | chimeric ImAb4 | WO2006037604 | 5677 |
| | | | | SEQ ID NO: 16 | |
| MUS32 | EAG1 | Heavy chain | HC-lmAb3-hum VH3- | WO2006037604 | 5678 |
| | | | 72 | SEQ ID NO: 20 | |
| MUS33 | EAG1 | Heavy chain | HC-lmAb4-hum VH4- | WO2006037604 | 5679 |
| | | | 59 | SEQ ID NO: 24 | |
| MUS34 | EAG1 | Heavy chain | HC-lmAb3-humVH3 | WO2006037604 | 5680 |
| | | | 23 | SEQ ID NO: 28 | |
| MUS35 | EAG1 | Heavy chain | HC-lmAb3-hum VH2 | WO2006037604 | 5681 |
| | | | 26 | SEQ ID NO: 32 | |
| MUS36 | EAG1 | Heavy chain | HC-lmAb4-hum VH1- | WO2006037604 | 5682 |
| | | | 3 | SEQ ID NO: 36 | |
| MUS37 | EAG1 | Heavy chain | ImAb4 | WO2006037604 | 5683 |
| | | | | SEQ ID NO: 4 | |
| MUS38 | EAG1 | Heavy chain | ImAb3 | WO2006037604 | 5684 |
| | | | | SEQ ID NO: 8 | |
| MUS39 | GDF-8 | Heavy chain | 358-22 | US20130287762 | 5685 |
| | | | | SEQ ID NO: 10 | |
| MUS40 | GDF-8 | Heavy chain | 358-11-M1 | US20130287762 | 5686 |
| | | | | SEQ ID NO: 16 | |
| MUS41 | GDF-8 | Heavy chain | 358-22-M1 | US20130287762 | 5687 |
| | | | | SEQ ID NO: 4 | |
| MUS42 | growth | Heavy chain | | | 5688 |
| | differentiation | | | | |
| | factor 8 | | | | |
| MUS43 | growth | Heavy chain | Domagrozumab | | 5689 |
| | differentiation | | | | |
| | factor 8 | | | | |
| MUS44 | MAG | Heavy chain | | | 5690 |
| MUS45 | MSTN | Heavy chain | H24L13, H24L16, | | 5691 |
| | | | H24L18, H24L14, | | |
| | | | H24L15, H24L17, | | |
| | | | H24L6, H24L11 | | |
| MUS46 | myostatin | Heavy chain | NI-204.11F11 | US20110256132 | 5692 |
| | | | | SEQ ID NO: 26 | |
| MUS47 | myostatin | Heavy chain | NI-204.67E12 | US20110256132 | 5693 |
| | | | | SEQ ID NO: 28 | |
| MUS48 | myostatin | Heavy chain | NI-204.6H1 | US20110256132 | 5694 |
| | | | | SEQ ID NO: 29 | |
| MUS49 | myostatin | Heavy chain | NI-204.6H1 | US20110256132 | 5695 |
| | | | | SEQ ID NO: 30 | |
| MUS50 | Myostatin | Heavy chain | 312-19, 312-19-MI | US20130142788 | 5696 |
| | | | | SEQ ID NO: 123 | |
| MUS51 | Myostatin | Heavy chain | 591-33. 591-33-M1 | US20130142788 | 5697 |
| | | | | SEQ ID NO: 125 | |
| MUS52 | Myostatin | Heavy chain | 114-41, 114-41-M1 | US20130142788 | 5698 |
| | | | | SEQ ID NO: 127 | |
| MUS53 | Myostatin | Heavy chain | 595-16, 595-16- M1 | US20130142788 | 5699 |
| | | | | SEQ ID NO: 138 | |
| MUS54 | Myostatin | Heavy chain | 591-37, 591-37-MI | US20130142788 | 5700 |
| | | | | SEQ ID NO: 139 | |
| MUS55 | Myostatin | Heavy chain | 358-11, 358-11-M1 | US20130142788 | 5701 |
| | | | | SEQ ID NO: 140 | |
| MUS56 | Myostatin | Heavy chain | 358-22, 358-22-M1 | US20130142788 | 5702 |
| | | | | SEQ ID NO: 141 | |
| MUS57 | Myostatin | Heavy chain | 597-120, 597-120-M1 | US20130142788 | 5703 |
| | | | | SEQ ID NO: 142 | |
| MUS58 | Myostatin | Heavy chain | 311-3 | US20130142788 | 5704 |
| | | | | SEQ ID NO: 143 | |
| MUS59 | Myostatin | Heavy chain | 311-3-MI | US20130142788 | 5705 |
| | | | | SEQ ID NO: 144 | |
| MUS60 | Myostatin | Heavy chain | 312-19-M1 | US20130142788 | 5706 |
| | | | | SEQ ID NO: 26 | |
| MUS61 | Myostatin | Heavy chain | 114-41 | US20130142788 | 5707 |
| | | | | SEQ ID NO: 30 | |
| MUS62 | Myostatin | Heavy chain | 311-3-M1 | US20130142788 | 5708 |
| | | | | SEQ ID NO: 35 | |
| MUS63 | Myostatin | Heavy chain | 312-19 | US20130142788 | 5709 |
| | | | | SEQ ID NO: 36 | |
| MUS64 | Myostatin | Heavy chain | 591-33 | US20130142788 | 5710 |
| | | | | SEQ ID NO: 38 | |
| MUS65 | Myostatin | Heavy chain | 591-33-M1 | US20130142788 | 5711 |
| | | | | SEQ ID NO: 39 | |
| MUS66 | Myostatin | Heavy chain | 312-56 | US20130142788 | 5712 |
| | | | | SEQ ID NO: 98 | |
| MUS67 | myostatin | Heavy chain | NJ-205.21G2 | US20130209489 | 5713 |
| | antagonists | | | SEQ ID NO: 11 | |
| MUS68 | myostatin | Heavy chain | NI-205.8A2 | US20130209489 | 5714 |
| | antagonists | | | SEQ ID NO: 12 | |
| MUS69 | myostatin | Heavy chain | NI-205.8A2 | US20130209489 | 5715 |
| | antagonists | | | SEQ ID NO: 13 | |
| MUS70 | myostatin | Heavy chain | NI-205.15F12 | US20130209489 | 5716 |
| | antagonists | | | SEQ ID NO: 14 | |
| MUS71 | myostatin | Heavy chain | NI-205.15F12 | US20130209489 | 5717 |
| | antagonists | | | SEQ ID NO: 15 | |
| MUS72 | myostatin | Heavy chain | NI-205.113C4 | US20130209489 | 5718 |
| | antagonists | | | SEQ ID NO: 16 | |
| MUS73 | myostatin | Heavy chain | NI-205.113C4 | US20130209489 | 5719 |
| | antagonists | | | SEQ ID NO: 17 | |
| MUS74 | myostatin | Heavy chain | NI-205.25F3 | US20130209489 | 5720 |
| | antagonists | | | SEQ ID NO: 18 | |
| MUS75 | myostatin | Heavy chain | NI-205.25F3 | US20130209489 | 5721 |
| | antagonists | | | SEQ ID NO: 19 | |
| MUS76 | NOGO | Heavy chain | H19L13 FL, H19L16 | US20140147435 | 5722 |
| | | | FL, H19L18 FL | SEQ ID NO: 92 | |
| MUS77 | NOGO | Heavy chain | H20L13 FL, H20L16 | US20140147435 | 5723 |
| | | | FL, H20L18 FL | SEQ ID NO: 93 | |
| MUS78 | NOGO | Heavy chain | H21L13 FL, H21L16 | US20140147435 | 5724 |
| | | | FL, H21L18 FL | SEQ ID NO: 94 | |
| MUS79 | NOGO | Heavy chain | H25L13 FL, H25L16 | US20140147435 | 5725 |
| | | | FL, H25L18 FL | SEQ ID NO: 98 | |
| MUS80 | NOGO | Heavy chain | H6L13 FL | US20140147435 | 5726 |
| | | | | SEQ ID NO: 27 | |
| MUS81 | NOGO | Heavy chain | H16L16 FL, H16L18 | US20140147435 | 5727 |
| | | | FL | SEQ ID NO: 31 | |
| MUS82 | NOGO | Heavy chain | H18L16 FL | US20140147435 | 5728 |
| | | | | SEQ ID NO: 33 | |
| MUS83 | Nogo receptor- | Heavy chain | 5B10 | US20090215691 | 5729 |
| | 1 | | | SEQ ID NO: 16 | |
| MUS84 | Nogo receptor- | Heavy chain | 5B10 | US20090215691 | 5730 |
| | 1 | | | SEQ ID NO: 18 | |
| MUS85 | RTN4 | Heavy chain | | SEQ ID NO: 38 | 5731 |
| | | | | U.S. Pat. No. | |
| | | | | 7,780,964 | |
| MUS86 | S1P4 | Heavy chain | | WO2015057939 | 5732 |
| | | | | SEQ ID NO: 39 | |
| MUS87 | trk-C (NT-3 | Heavy chain | 2250 | U.S. Pat. No. | 5733 |
| | trkC ligand) | | | 7,615,383 | |
| | | | | SEQ ID NO: 42 | |
| MUS88 | trk-C (NT-3 | Heavy chain | 2253 | U.S. Pat. No. | 5734 |
| | trkC ligand) | | | 7,615,383 | |
| | | | | SEQ ID NO: 43 | |
| MUS89 | trk-C (NT-3 | Heavy chain | 2256 | U.S. Pat. No. | 5735 |
| | trkC ligand) | | | 7,615,383 | |
| | | | | SEQ ID NO: 44 | |
| MUS90 | trk-C (NT-3 | Heavy chain | 6.1.2 | U.S. Pat. No. | 5736 |
| | trkC ligand) | | | 7,615,383 | |
| | | | | SEQ ID NO: 45 | |
| MUS91 | trk-C (NT-3 | Heavy chain | 6.4.1 | U.S. Pat. No. | 5737 |
| | trk(C ligand) | | | 7,615,383 | |
| | | | | SEQ ID NO: 46 | |
| MUS92 | trk-C (NT-3 | Heavy chain | 2345 | U.S. Pat. No. | 5738 |
| | trkC ligand) | | | 7,615,383 | |
| | | | | SEQ ID NO: 47 | |
| MUS93 | trk-C (NT-3 | Heavy chain | 2349 | U.S. Pat. No. | 5739 |
| | trkC ligand) | | | 7,615,383 | |
| | | | | ISEQ D NO: 48 | |
| MUS94 | myostatin | Heavy chain | NI-205.87E7 | US20130209489 | 5740 |
| | antagonists | consensus | | SEQ ID NO: 20 | |
| MUS95 | GDF-8 | Heavy chain | | U.S. Pat. No. | 5741 |
| | | constant region | | 8,956,608 | |
| | | | | SEQ ID NO: 19 | |
| MUS96 | many - growth | Heavy chain fusion | H19L13, H19L16, | U.S. Pat. No. | 5742 |
| | factors (to | protein | H19L18, H19L14, | 8,053,569 | |
| | increase | | H19L15, H19L17, | SEQ ID NO: 25 | |
| | transport across | | H19L6, H19L11 | | |
| | BBB) | | | | |
| MUS97 | many - growth | Heavy chain fusion | H20L13, H20L16, | U.S. Pat. No. | 5743 |
| | factors (to | protein | H20L18, H20L14, | 8,053,569 | |
| | increase | | H20L15, H20L17, | SEQ ID NO: 28 | |
| | transport across | | H20L6, H20L11 | | |
| | BBB) | | | | |
| MUS98 | many - growth | Heavy chain fusion | H22L13, H22L16, | U.S. Pat. No. | 5744 |
| | factors (to | protein | H22L18, H22L14, | 8,053,569 | |
| | increase | | H22L15, H22L17, | SEQ ID NO: 34 | |
| | transport across | | H22L6, H22L11 | | |
| | BBB) | | | | |
| MUS99 | many - growth | Heavy chain fusion | H5L11, H6L11, | U.S. Pat. No. | 5745 |
| | factors (to | protein | H14L11, H15L11, | 8,053,569 | |
| | increase | | H16L11, H17L11, | SEQ ID NO: 24 | |
| | transport across | | H18L11, H19L11, | | |
| | BBB) | | H20L11, H21L11, | | |
| | | | H22L11, H23L11, | | |
| | | | H24L11, H25L11, | | |
| | | | H700L11 | | |
| MUS100 | NOGO | Heavy chain | 2A10 construct | WO2007003421 | 5746 |
| | | humanized | | SEQ ID NO: 79 | |
| | | construct H1 | | | |
| MUS101 | NOGO | Heavy chain | 2A10 construct | WO2007003421 | 5747 |
| | | humanized | | SEQ ID NO: 29 | |
| | | construct H14 | | | |
| MUS102 | NOGO | Heavy chain | 2A10 construct | WO2007003421 | 5748 |
| | | humanized | | SEQ ID NO: 30 | |
| | | construct H15 | | | |
| MUS103 | NOGO | Heavy chain | 2A10 construct | WO2007003421 | 5749 |
| | | humanized | | SEQ ID NO: 31 | |
| | | construct H16 | | | |
| MUS104 | NOGO | Heavy chain | 2A10 construct | WO2007003421 | 5750 |
| | | humanized | | SEQ ID NO: 32 | |
| | | construct H17 | | | |
| MUS105 | NOGO | Heavy chain | 2A10 construct | WO2007003421 | 5751 |
| | | humanized | | SEQ ID NO: 33 | |
| | | construct H18 | | | |
| MUS106 | NOGO | Heavy chain | 2A10 construct | WO2007003421 | 5752 |
| | | humanized | | SEQ ID NO: 92 | |
| | | construct H19 | | | |
| MUS107 | NOGO | Heavy chain | 2A10 construct | WO2007003421 | 5753 |
| | | humanized | | SEQ ID NO: 93 | |
| | | construct H20 | | | |
| MUS108 | NOGO | Heavy chain | 2A10 construct | WO2007003421 | 5754 |
| | | humanized | | SEQ ID NO: 94 | |
| | | construct H21 | | | |
| MUS109 | NOGO | Heavy chain | 2A10 construct | WO2007003421 | 5755 |
| | | humanized | | SEQ ID NO: 95 | |
| | | construct H22 | | | |
| MUS110 | NOGO | Heavy chain | 2A10 construct | WO2007003421 | 5756 |
| | | humanized | | SEQ ID NO: 96 | |
| | | construct H23 | | | |
| MUS111 | NOGO | Heavy chain | 2A10 construct | WO2007003421 | 5757 |
| | | humanized | | SEQ ID NO: 97 | |
| | | construct H24 | | | |
| MUS112 | NOGO | Heavy chain | 2A10 construct | WO2007003421 | 5758 |
| | | humanized | | SEQ ID NO: 98 | |
| | | construct H25 | | | |
| MUS113 | NOGO | Heavy chain | 2A10 construct | WO2007003421 | 5759 |
| | | humanized | | SEQ ID NO: 26 | |
| | | construct H5 | | | |
| MUS114 | NOGO | Heavy chain | 2A10 construct | WO2007003421 | 5760 |
| | | humanized | | SEQ ID NO: 27 | |
| | | construct H6 | | | |
| MUS115 | NOGO | Heavy chain | 2A10 construct | WO2007003421 | 5761 |
| | | humanized | | SEQ ID NO: 28 | |
| | | construct H700 | | | |
| MUS116 | RTN4 (NOGO) | Heavy chain IgG4, | Atinumab | U.S. Pat. No. | 5762 |
| | | immunomodulator | | 8,163,285 | |
| | | | | SEQ ID NO: 24 | |
| MUS117 | amyloid | Heavy chain | F11G3 | U.S. Pat. No. | 5763 |
| | oligomers | variable region | | 9,125,846 | |
| | | | | SEQ ID NO: 11 | |
| MUS118 | differentiation | Heavy chain | H8L4. H8L5, H8L6 | US20140023638 | 5764 |
| | factor 8 | variable region | | SEQ ID NO: 17 | |
| | (GDF8) | | | | |
| MUS119 | DR6 and P75 | Heavy chain | M62-B02 | WO2010062904 | 5765 |
| | | variable region | | SEQ ID NO: 47 | |
| MUS120 | DR6 and P75 | Heavy chain | M63-E10 | WO2010062904 | 5766 |
| | | variable region | | SEQ ID NO: 57 | |
| MUS121 | DR6 and P75 | Heavy chain | M66-B03 | WO2010062904 | 5767 |
| | | variable region | | SEQ ID NO: 67 | |
| MUS122 | DR6 and P75 | Heavy chain | M50-H01 | WO2010062904 | 5768 |
| | | variable region | | SEQ ID NO: 7 | |
| MUS123 | DR6 and P75 | Heavy chain | M67-G02 | WO2010062904 | 5769 |
| | | variable region | | SEQ ID NO: 77 | |
| MUS124 | DR6 and P75 | Heavy chain | M72-F03 | WO2010062904 | 5770 |
| | | variable region | | SEQ ID NO: 87 | |
| MUS125 | DR6 and P75 | Heavy chain | M73-C04 | WO2010062904 | 5771 |
| | | variable region | | SEQ ID NO: 97 | |
| MUS126 | DR6 and P75 | Heavy chain | 1P1D6.3 | WO2010062904 | 5772 |
| | | variable region | | SEQ ID NO: 107 | |
| MUS127 | DR6 and P75 | Heavy chain | 1P2F2.1 | WO2010062904 | 5773 |
| | | variable region | | SEQ ID NO: 117 | |
| MUS128 | DR6 and P75 | Heavy chain | IP5D10.2 | WO2010062904 | 5774 |
| | | variable region | | SEQ ID NO: 127 | |
| MUS129 | DR6 and P75 | Heavy chain | M51-H09 | WO2010062904 | 5775 |
| | | variable region | | SEQ ID NO: 17 | |
| MUS130 | DR6 and P75 | Heavy chain | M53-E04 | WO2010062904 | 5776 |
| | | variable region | | SEQ ID NO: 27 | |
| MUS131 | DR6 and P75 | Heavy chain | M53-F04 | WO2010062904 | 5777 |
| | | variable region | | SEQ ID NO: 37 | |
| MUS132 | GDF-8 | Heavy chain | 595-16 | U.S. Pat. No. | 5778 |
| | | variable region | | 8,956,608 | |
| | | | | SEQ ID NO: 26 | |
| MUS133 | GDF-8 | Heavy chain | 12A5-10HC | U.S. Pat. No. | 5779 |
| | | variable region | | 8,956,608 | |
| | | | | SEQ ID NO: 7 | |
| MUS134 | growth | Heavy chain | | U.S. Pat. No. | 5780 |
| | differentiation | variable region | | 8,840,894 | |
| | factor 8 | | | SEQ ID NO: 360 | |
| MUS135 | LPG | Heavy chain | #7 | U.S. Pat. No. | 5781 |
| | (lysophos- | variable region | | 8,591,902 | |
| | phatidyl- | | | SEQ ID NO: 18 | |
| | glucoside) | | | | |
| MUS136 | LPG | Heavy chain | #15 | U.S. Pat. No. | 5782 |
| | (lysophos- | variable region | | 8,591,902 | |
| | phatidyl- | | | SEQ ID NO: 8 | |
| | glucoside) | | | | |
| MUS137 | MAG | Heavy chain | | U.S. Pat. No. | 5783 |
| | | variable region | | 8,071,731 | |
| | | | | SEQ ID NO: 13 | |
| MUS138 | MAG | Heavy chain | | U.S. Pat. No. | 5784 |
| | | variable region | | 8,071,731 | |
| | | | | SEQ ID NO: 14 | |
| MUS139 | MAG | Heavy chain | | U.S. Pat. No. | 5785 |
| | | variable region | | 8,071,731 | |
| | | | | SEQ ID NO: 15 | |
| MUS140 | MAI (myelin | Heavy chain | | WO2013158748 | 5786 |
| | associated | variable region | | SEQ ID NO: 1 | |
| | inhibitor) | | | | |
| MUS141 | MAI (myelin | Heavy chain | | WO2013158748 | 5787 |
| | associated | variable region | | SEQ ID NO: 17 | |
| | inhibitor) | | | | |
| MUS142 | myostatin | Heavy chain | NI-204.7B3 | SEQ ID 6 WO | 5788 |
| | | variable region | | 2006107611 | |
| MUS143 | myostatin | Heavy chain | NI-204.10A8 | US20110256132 | 5789 |
| | | variable region | | SEQ ID NO: 14 | |
| MUS144 | myostatin | Heavy chain | NI-204.10D12 | US20110256132 | 5790 |
| | | variable region | | SEQ ID NO: 19 | |
| MUS145 | myostatin | Heavy chain | NI-204.10D12 | US20110256132 | 5791 |
| | | variable region | | SEQ ID NO: 20 | |
| MUS146 | myostatin | Heavy chain | NI-104.12G7 | US20110256132 | 5792 |
| | | variable region | | SEQ ID NO: 22 | |
| MUS147 | Inyostatin | Heavy chain | NI-204.10A8 | US20110256132 | 5793 |
| | | variable region | | SEQ ID NO: 23 | |
| MUS148 | myostatin | Heavy chain | NI-104.12G7 | US20110256132 | 5794 |
| | | variable region | | SEQ ID NO: 25 | |
| MUS149 | myostatin | Heavy chain | 312-56 | US20110256132 | 5795 |
| | | variable region | | SEQ ID NO: 8 | |
| MUS150 | NOGO | Heavy chain | H20L13, H20L16, | US20140147435 | 5796 |
| | | variable region | H20L18, H20L14, | SEQ ID NO: 86 | |
| | | | H20L15, H20L17, | | |
| | | | H20L6, H20L11 | | |
| MUS151 | NOGO | Heavy chain | H21L13, H21L16, | US20140147435 | 5797 |
| | | variable region | H21L18, H21L14, | SEQ ID NO: 87 | |
| | | | H21L15, H21L17, | | |
| | | | H21L6, H21L11 | | |
| MUS152 | NOGO | Heavy chain | H22L13, H22L16, | US20140147435 | 5798 |
| | | variable region | H22L18, H22L14, | SEQ ID NO: 88 | |
| | | | H22L15, H22L17, | | |
| | | | H22L6, H22L11 | | |
| MUS153 | NOGO | Heavy chain | H23L13, H23L16, | US20140147435 | 5799 |
| | | variable region | H23L18, H23L14, | SEQ ID NO: 89 | |
| | | | H23L15, H23L17, | | |
| | | | H23L6, H23L11 | | |
| MUS154 | NOGO | Heavy chain | H24L13, H24L16, | US20140147435 | 5800 |
| | | variable region | H24L18, H24L14, | SEQ ID NO: 90 | |
| | | | H24L15, H24L17, | | |
| | | | H24L6, H24L11 | | |
| MUS155 | NOGO | Heavy chain | H25L13, H25L16, | US20140147435 | 5801 |
| | | variable region | H25L18, H25L14, | SEQ ID NO: 91 | |
| | | | H25L15, H25L17, | | |
| | | | H25L6, H25L11 | | |
| MUS156 | NOGO | Heavy chain | H5L13, H5L16, | US20140147435 | 5802 |
| | | variable region | H5L18, H5L14, | SEQ ID NO: 11 | |
| | | | H5L15, H5L 17, H5L6, | | |
| | | | H5L11 | | |
| MUS157 | NOGO | Heavy chain | H6L13, H6L16, | US20140147435 | 5803 |
| | | variable region | H6L18, H6L14, | SEQ ID NO: 12 | |
| | | | H6L15, H6L17, H6L6 | | |
| MUS158 | NOGO | Heavy chain | H700L13, H700L16, | US20140147435 | 5804 |
| | | variable region | H700L18, H700L14, | SEQ ID NO: 13 | |
| | | | H700L15, H700L17, | | |
| | | | H700L6, H700L11 | | |
| MUS159 | NOGO | Heavy chain | H14L13, H14L16, | US20140147435 | 5805 |
| | | variable region | H14L18, H14L14, | SEQ ID NO: 14 | |
| | | | H14L15, H14L17, | | |
| | | | H14L6, H14L11 | | |
| MUS160 | NOGO | Heavy chain | H15L13, H15L16, | US20140147435 | 5806 |
| | | variable region | H15L18, H15L14, | SEQ ID NO: 15 | |
| | | | H15L15, H15L17, | | |
| | | | H15L6, H15L11 | | |
| MUS161 | NOGO | Heavy chain | H16L13, H16L16, | US20140147435 | 5807 |
| | | variable region | H16L18, H16L14, | SEQ ID NO: 16 | |
| | | | H16L15, H16L17, | | |
| | | | H16L6, H16L11 | | |
| MUS162 | NOGO | Heavy chain | H17L13, H17L16, | US20140147435 | 5808 |
| | | variable region | H17L18, H17L14, | SEQ ID NO: 17 | |
| | | | H17L15, H17L17, | | |
| | | | H17L6, H17L11 | | |
| MUS163 | NOGO | Heavy chain | H18L13, H18L16, | US20140147435 | 5809 |
| | | variable region | H18L18, H18L14, | SEQ ID NO: 18 | |
| | | | H18L15, H18L17, | | |
| | | | H18L6, H18L11 | | |
| MUS164 | NOGO | Heavy chain | H1L13, H1L16, | US20140147435 | 5810 |
| | | variable region | H1L18, H1L14, | SEQ ID NO: 77 | |
| | | | H1L15, H1L17, H1L6 | | |
| MUS165 | NOGO | Heavy chain | H19L13, H19L16, | US20140147435 | 5811 |
| | | variable region | H19L18, H19L14, | SEQ ID NO: 85 | |
| | | | H19L15, H19L17, | | |
| | | | H19L6, H19L11 | | |
| MUS166 | Nogo-66 | Heavy chain | Antibody clone 50 | US20140065155 | 5812 |
| | | variable region | | SEQ ID NO: 3 | |
| MUS167 | Nogo-66 | Heavy chain | Antibody clone 51 | US20140065155 | 5813 |
| | | variable region | | SEQ ID NO: 5 | |
| MUS168 | NogoA/NiG | Heavy chain | 6A3-Ig4 | WO2009056509 | 5814 |
| | | variable region | | SEQ ID NO: 24 | |
| MUS169 | NogoA/NiG | Heavy chain | 6A3-IgG1 | WO2009056509 | 5815 |
| | | variable region | | SEQ ID NO: 4 | |
| MUS170 | RGM A | Heavy chain | 5F9.1-GL | US20150183871 | 5816 |
| | | variable region | | SEQ ID NO: 35 | |
| MUS171 | RGM A | Heavy chain | 5F9.2-GL | US20150183871 | 5817 |
| | | variable region | | SEQ ID NO: 36 | |
| MUS172 | RGM A | Heavy chain | 5F9.3-GL | US20150183871 | 5818 |
| | | variable region | | SEQ ID NO: 37 | |
| MUS173 | RGM A | Heavy chain | 5F9.4-GL | US20150183871 | 5819 |
| | | variable region | | SEQ ID NO: 38 | |
| MUS174 | RGM A | Heavy chain | 5F9.5-GL | US20150183871 | 5820 |
| | | variable region | | SEQ ID NO: 39 | |
| MUS175 | RGM A | Heavy chain | 5F9.6-GL | US20150183871 | 5821 |
| | | variable region | | SEQ ID NO: 40 | |
| MUS176 | RGM A | Heavy chain | 5F9.7-GL | US20150183871 | 5822 |
| | | variable region | | SEQ ID NO: 41 | |
| MUS177 | RGM A | Heavy chain | 5F9.8-GL | US20150183871 | 5823 |
| | | variable region | | SEQ ID NO: 42 | |
| MUS178 | RGM A | Heavy chain | 5F9.9-GL | US20150183871 | 5824 |
| | | variable region | | SEQ ID NO: 43 | |
| MUS179 | RGM A | Heavy chain | h5F9.1, h5F9.1, | US20150183871 | 5825 |
| | | variable region | h5F9.1, h5F9.1, | SEQ ID NO: 47 | |
| | | | h5F9.1, h5F9.2, | | |
| | | | h5F9.3 | | |
| MUS180 | RGM A | Heavy chain | h5F9.3, h5F9.9, | US20150183871 | 5826 |
| | | variable region | h5F9.25 | SEQ ID NO: 53 | |
| MUS181 | RGM A | Heavy chain | h5F9.4, h5F9.10, | US20150183871 | 5827 |
| | | variable region | h5F9.26 | SEQ ID NO: 54 | |
| MUS182 | RGMa | Heavy chain | AE12-1 | US20140023659 | 5828 |
| | | variable region | | SEQ ID NO: 1 | |
| MUS183 | R.GMa | Heavy chain | AE12-20 | US20140023659 | 5829 |
| | | variable region | | SEQ ID NO: 107 | |
| MUS184 | RGMa | Heavy chain | AE12-21 | US20140023659 | 5830 |
| | | variable region | | SEQ ID NO: 115 | |
| MUS185 | RGMa | Heavy chain | AE12-23 | US20140023659 | 5831 |
| | | variable region | | SEQ ID NO: 123 | |
| MUS186 | RGMa | Heavy chain | AE12-24 | US20140023659 | 5832 |
| | | variable region | | SEQ ID NO: 131 | |
| MUS187 | R.GMa | Heavy chain | AE12-3 | US20140023659 | 5833 |
| | | variable region | | SEQ ID NO: 17 | |
| MUS188 | RGMa | Heavy chain | AE12-4 | US20140023659 | 5834 |
| | | variable region | | SEQ ID NO: 25 | |
| MUS189 | RGMa | Heavy chain | AE12-5 | US20140023659 | 5835 |
| | | variable region | | SEQ ID NO: 33 | |
| MUS190 | RGMa | Heavy chain | AE12-6 | US20140023659 | 5836 |
| | | variable region | | SEQ ID NO: 41 | |
| MUS191 | RGMa | Heavy chain | AE12-7 | US20140023659 | 5837 |
| | | variable region | | SEQ ID NO: 49 | |
| MUS192 | RGMa | Heavy chain | AE12-8 | US20140023659 | 5838 |
| | | variable region | | SEQ ID NO: 57 | |
| MUS193 | RGMa | Heavy chain | AE12-2 | US20140023659 | 5839 |
| | | variable region | | SEQ ID NO: 9 | |
| MUS194 | RGMa | Heavy chain | AE12-13 | US20140023659 | 5840 |
| | | variable region | | SEQ ID NO: 91 | |
| MUS195 | RGMa | Heavy chain | AE12-15 | US20140023659 | 5841 |
| | | variable region | | SEQ ID NO: 99 | |
| MUS196 | SIP4 | Heavy chain | | WO2015057939 | 5842 |
| | | variable region | | SEQ ID NO: 7 | |
| MUS197 | SOD1 | Heavy chain | NI205.19G5 | US20140301945 | 5843 |
| | | variable region | | SEQ ID NO: 12 | |
| MUS198 | SOD1 | Heavy chain | | US20140301945 | 5844 |
| | | variable region | | SEQ ID NO: 16 | |
| MUS199 | SOD1 | Heavy chain | | US20140301945 | 5845 |
| | | variable region | | SEQ ID NO: 20 | |
| MUS200 | SOD1 | Heavy chain | | US20140301945 | 5846 |
| | | variable region | | SEQ ID NO: 24 | |
| MUS201 | SOD1 | Heavy chain | Landogrozumab, | US20140301945 | 5847 |
| | | variable region | LY2495655, LY- | SEQ ID NO: 28 | |
| | | | 2495655 | | |
| MUS202 | SOD1 | Heavy chain | 2A10 construct | US20140301945 | 5848 |
| | | variable region | | SEQ ID NO: 32 | |
| MUS203 | SOD1 | Heavy chain | 2A10 construct | US20140301945 | 5849 |
| | | variable region | | SEQ ID NO: 36 | |
| MUS204 | SOD1 | Heavy chain | NI205.1A9 | US20140301945 | 5850 |
| | | variable region | | SEQ ID NO: 4 | |
| MUS205 | SOD1 | Heavy chain | 2A10 construct | US20140301945 | 5851 |
| | | variable region | | SEQ ID NO: 40 | |
| MUS206 | SOD1 | Heavy chain | 2A10 construct | US20140301945 | 5852 |
| | | variable region | | SEQ ID NO: 44 | |
| MUS207 | SOD1 | Heavy chain | 2A10 construct | US20140301945 | 5853 |
| | | variable region | | SEQ ID NO: 48 | |
| MUS208 | SOD1 | Heavy chain | NI205.14W3 | US20140301945 | 5854 |
| | | variable region | | SEQ ID NO: 8 | |
| MUS209 | SOD1 | Heavy chain | NI-205.87E7 | U.S. Pat. No. | 5855 |
| | | variable region | | 9,109,037 | |
| | | | | SEQ ID NO: 1 | |
| MUS210 | SOD1 | Heavy chain | NI205.9E12 | U.S. Pat. No. | 5856 |
| | | variable region | | 9,109,037 | |
| | | | | SEQ ID NO: 107 | |
| MUS211 | SOD1 | Heavy chain | NI205.9E12 | U.S. Pat. No. | 5857 |
| | | variable region | | 9,109,037 | |
| | | | | SEQ ID NO: 113 | |
| MUS212 | SOD1 | Heavy chain | NI205.98H6 | U.S. Pat. No. | 5858 |
| | | variable region | | 9,109,037 | |
| | | | | SEQ ID NO: 129 | |
| MUS213 | SOD1 | Heavy chain | NI205.98H6 | U.S. Pat. No. | 5859 |
| | | variable region | | 9,109,037 | |
| | | | | SEQ ID NO: 131 | |
| MUS214 | SOD1 | Heavy chain | NI205.10D3 | U.S. Pat. No. | 5860 |
| | | variable region | | 9,109,037 | |
| | | | | SEQ ID NO: 147 | |
| MUS215 | SOD1 | Heavy chain | NI205.10D3 | U.S. Pat. No. | 5861 |
| | | variable region | | 9,109,037 | |
| | | | | SEQ ID NO: 149 | |
| MUS216 | SOD1 | Heavy chain | NI205.44B22 | U.S. Pat. No. | 5862 |
| | | variable region | | 9,109,037 | |
| | | | | SEQ ID NO: 165 | |
| MUS217 | SOD1 | Heavy chain | NI205.44B22 | U.S. Pat. No. | 5863 |
| | | variable region | | 9,109,037 | |
| | | | | SEQ ID NO: 167 | |
| MUS218 | SOD1 | Heavy chain | NI-205.21G1 | U.S. Pat. No. | 5864 |
| | | variable region | | 9,109,037 | |
| | | | | SEQ ID NO: 17 | |
| MUS219 | SOD1 | Heavy chain | NI205.38H2 | U.S. Pat. No. | 5865 |
| | | variable region | | 9,109,037 | |
| | | | | SEQ ID NO: 183 | |
| MUS220 | SOD1 | Heavy chain | NI-205.21G1 | U.S. Pat. No. | 5866 |
| | | variable region | | 9,109,037 | |
| | | | | SEQ ID NO: 19 | |
| MUS221 | SOD1 | Heavy chain | NI205.38H2 | U.S. Pat. No. | 5867 |
| | | variable region | | 9,109,037 | |
| | | | | SEQ ID NO: 201 | |
| MUS222 | SOD1 | Heavy chain | NI205.36D5 | U.S. Pat. No. | 5868 |
| | | variable region | | 9,109,037 | |
| | | | | SEQ ID NO: 217 | |
| MUS223 | SOD1 | Heavy chain | NI-205.68G5 | U.S. Pat. No. | 5869 |
| | | variable region | | 9,109,037 | |
| | | | | SEQ ID NO: 35 | |
| MUS224 | SOD1 | Heavy chain | NI-205.68G5 | U.S. Pat. No. | 5870 |
| | | variable region | | 9,109,037 | |
| | | | | SEQ ID NO: 37 | |
| MUS225 | SOD1 | Heavy chain | NI-205.20A1 | U.S. Pat. No. | 5871 |
| | | variable region | | 9,109,037 | |
| | | | | SEQ ID NO: 53 | |
| MUS226 | SOD1 | Heavy chain | NI-205.20A1 | U.S. Pat. No. | 5872 |
| | | variable region | | 9,109,037 | |
| | | | | SEQ ID NO: 55 | |
| MUS227 | SOD1 | Heavy chain | NI205.41D1 | U.S. Pat. No. | 5873 |
| | | variable region | | 9,109,037 | |
| | | | | SEQ ID NO: 71 | |
| MUS228 | SOD1 | Heavy chain | NI205.41D1 | U.S. Pat. No. | 5874 |
| | | variable region | | 9,109,037 | |
| | | | | SEQ ID NO: 73 | |
| MUS229 | SOD1 | Heavy chain | NI205.29E11 | U.S. Pat. No. | 5875 |
| | | variable region | | 9,109,037 | |
| | | | | SEQ ID NO: 89 | |
| MUS230 | SOD1 | Heavy chain | NI205.29E11 | U.S. Pat. No. | 5876 |
| | | variable region | | 9,109,037 | |
| | | | | SEQ ID NO: 91 | |
| MUS231 | TDP-43 | Heavy chain | 2A10 construct | US20140255304 | 5877 |
| | | variable region | | SEQ ID NO: 1 | |
| MUS232 | TDP-43 | Heavy chain | 2A10 construct | US20140255304 | 5878 |
| | | variable region | | SEQ ID NO: 10 | |
| MUS233 | TDP-43 | Heavy chain | 2A10 construct | US20140255304 | 5879 |
| | | variable region | | SEQ ID NO: 130 | |
| MUS234 | TDP-43 | Heavy chain | 2A10 construct | US20140255304 | 5880 |
| | | variable region | | SEQ ID NO: 138 | |
| MUS235 | TDP-43 | Heavy chain | 2A10 construct | US20140255304 | 5881 |
| | | variable region | | SEQ ID NO: 146 | |
| MUS236 | TDP-43 | Heavy chain | 2A10 construct | US20140255304 | 5882 |
| | | variable region | | SEQ ID NO: 151 | |
| MUS237 | TDP-43 | Heavy chain | 2A10 construct | US20140255304 | 5883 |
| | | variable region | | SEQ ID NO: 159 | |
| MUS238 | TDP-43 | Heavy chain | 2A10 construct | US20140255304 | 5884 |
| | | variable region | | SEQ ID NO: 167 | |
| MUS239 | TDP-43 | Heavy chain | 2A10 construct | US20140255304 | 5885 |
| | | variable region | | SEQ ID NO: 175 | |
| MUS240 | TDP-43 | Heavy chain | 2A10 construct | US20140255304 | 5886 |
| | | variable region | | SEQ ID NO: 18 | |
| MUS241 | TDP-43 | Heavy chain | H6L13 FL, H19L13 | US20140255304 | 5887 |
| | | variable region | FL, H20L13 FL, | SEQ ID NO: 183 | |
| | | | H21L13 FL, H25L13 | | |
| | | | FL | | |
| MUS242 | TDP-43 | Heavy chain | H16L18 FL, H19L18 | US20140255304 | 5888 |
| | | variable region | FL, H20L 18 FL, | SEQ ID NO: 191 | |
| | | | H21L18 FL, H25L18 | | |
| | | | FL | | |
| MUS243 | TDP-43 | Heavy chain | H18L16 FL | US20140255304 | 5889 |
| | | variable region | | SEQ ID NO: 199 | |
| MUS244 | TDP-43 | Heavy chain | H6L13, H6L16, | US20140255304 | 5890 |
| | | variable region | H6L18, H6L14, | SEQ ID NO: 207 | |
| | | | H6L15, H6L17, H6L6 | | |
| MUS245 | TDP-43 | Heavy chain | H14L13, H14L16, | US20140255304 | 5891 |
| | | variable region | H14L18, H14L14, | SEQ ID NO: 215 | |
| | | | H14L15, H14L17, | | |
| | | | H1416, H14L11 | | |
| MUS246 | TDP-43 | Heavy chain | H16L13, H16L16, | US20140255304 | 5892 |
| | | variable region | H16L18, H16L14, | SEQ ID NO: 223 | |
| | | | H16L15, H16L17, | | |
| | | | H16L6, H16L11 | | |
| MUS247 | TDP-43 | Heavy chain | H18L13, H18L16, | US20140255304 | 5893 |
| | | variable region | H18L.18, H18L14, | SEQ ID NO: 231 | |
| | | | H18L15, H18L17, | | |
| | | | H18L6, H18L11 | | |
| MUS248 | TDP-43 | Heavy chain | H1L13, H5L13, | US20140255304 | 5894 |
| | | variable region | H6L13. H14L13, | SEQ ID NO: 239 | |
| | | | H15L13, H16L13, | | |
| | | | H17L13, H18L13, | | |
| | | | H19L13, H20L13, | | |
| | | | H21L13, H22L13, | | |
| | | | H23L.13, H24L13, | | |
| | | | H25L13, H700L13 | | |
| MUS249 | TDP-43 | Heavy chain | H1L15, H5L15, | US20140255304 | 5895 |
| | | variable region | H6L15, H14L15, | SEQ ID NO: 247 | |
| | | | H15L15, H16L15, | | |
| | | | H17L15, H18L15, | | |
| | | | H19L15, H20L15, | | |
| | | | H21L15, H22L15, | | |
| | | | H23L15, H24L15, | | |
| | | | H25L15, H700L15 | | |
| MUS250 | TDP-43 | Heavy chain | H1L17, H5L17, | US20140255304 | 5896 |
| | | variable region | H6L17, H14L17, | SEQ ID NO: 255 | |
| | | | H15L17, H16L17, | | |
| | | | H17L17, H18L17, | | |
| | | | H19L17, H20L17, | | |
| | | | H21L17, H22L17, | | |
| | | | H23L17, H24L17, | | |
| | | | H25L17, H700L17 | | |
| MUS251 | TDP-43 | Heavy chain | 2A10 construct | US20140255304 | 5897 |
| | | variable region | | SEQ ID NO: 26 | |
| MUS252 | TDP-43 | Heavy chain | H16L16 FL, H16L18 | US20140255304 | 5898 |
| | | variable region | FL | SEQ ID NO: 263 | |
| MUS253 | TDP-43 | Heavy chain | 2A10 construct | US20140255304 | 5899 |
| | | variable region | | SEQ ID NO: 35 | |
| MUS254 | TDP-43 | Heavy chain | 2A10 construct | US20140255304 | 5900 |
| | | variable region | | SEQ ID NO: 45 | |
| MUS255 | TDP-43 | Heavy chain | 2A10 construct | US20140255304 | 5901 |
| | | variable region | | SEQ ID NO: 53 | |
| MUS256 | TDP-43 | Heavy chain | 2A10 construct | US20140255304 | 5902 |
| | | variable region | | SEQ ID NO: 61 | |
| MUS257 | TDP-43 | Heavy chain | 2A10 construct | US20140255304 | 5903 |
| | | variable region | | SEQ ID NO: 69 | |
| MUS258 | TDP-43 | Heavy chain | 2A10 construct | US20140255304 | 5904 |
| | | variable region | | SEQ ID NO: 77 | |
| MUS259 | TDP-43 | Heavy chain | 2A10 construct | US20140255304 | 5905 |
| | | variable region | | SEQ ID NO: 87 | |
| MUS260 | trkC | Heavy chain | | US20070031418 | 5906 |
| | | variable region | | SEQ ID NO: 1 | |
| MUS261 | NOGO | Heavy chain | 2A10 construct | WO2007003421 | 5907 |
| | | variable region | | SEQ ID NO: 77 | |
| | | humanized | | | |
| | | construct H1 | | | |
| MUS262 | NOGO | Heavy chain | 2A10 construct | WO2007003421 | 5908 |
| | | variable region | | SEQ ID NO: 14 | |
| | | humanized | | | |
| | | construct H14 | | | |
| MUS263 | NOGO | Heavy chain | 2A10 construct | WO2007003421 | 5909 |
| | | variable region | | SEQ ID NO: 15 | |
| | | humanized | | | |
| | | construct H15 | | | |
| MUS264 | NOGO | Heavy chain | 2A10 construct | WO2007003421 | 5910 |
| | | variable region | | SEQ ID NO: 16 | |
| | | humanized | | | |
| | | construct H16 | | | |
| MUS265 | NOGO | Heavy chain | 2A10 construct | WO2007003421 | 5911 |
| | | variable region | | SEQ ID NO: 17 | |
| | | humanized | | | |
| | | construct H17 | | | |
| MUS266 | NOGO | Heavy chain | 2A10 construct | WO2007003421 | 5912 |
| | | variable region | | SEQ ID NO: 18 | |
| | | humanized | | | |
| | | construct H18 | | | |
| MUS267 | NOGO | Heavy chain | 2A10 construct | WO2007003421 | 5913 |
| | | variable region | | SEQ ID NO: 85 | |
| | | humanized | | | |
| | | construct H19 | | | |
| MUS268 | NOGO | Heavy chain | 2A10 construct | WO2007003421 | 5914 |
| | | variable region | | SEQ ID NO: 86 | |
| | | humanized | | | |
| | | construct H20 | | | |
| MUS269 | NOGO | Heavy chain | 2A10 construct | WO2007003421 | 5915 |
| | | variable region | | SEQ ID NO: 87 | |
| | | humanized | | | |
| | | construct H21 | | | |
| MUS270 | NOGO | Heavy chain | 2A10 construct | WO2007003421 | 5916 |
| | | variable region | | SEQ ID NO: 88 | |
| | | humanized | | | |
| | | construct H22 | | | |
| MUS271 | NOGO | Heavy chain | 2A10 construct | WO2007003421 | 5917 |
| | | variable region | | SEQ ID NO: 89 | |
| | | humanized | | | |
| | | construct H23 | | | |
| MUS272 | NOGO | Heavy chain | 2A10 construct | WO2007003421 | 5918 |
| | | variable region | | SEQ ID NO: 90 | |
| | | humanized | | | |
| | | construct H24 | | | |
| MUS273 | NOGO | Heavy chain | 2A10 construct | WO2007003421 | 5919 |
| | | variable region | | SEQ ID NO: 91 | |
| | | humanized | | | |
| | | construct I-25 | | | |
| MUS274 | NOGO | Heavy chain | 2A10 construct | WO2007003421 | 5920 |
| | | variable region | | SEQ ID NO: 11 | |
| | | humanized | | | |
| | | construct H5 | | | |
| MUS275 | NOGO | Heavy chain | 2A10 construct | WO2007003421 | 5921 |
| | | variable region | | SEQ ID NO: 12 | |
| | | humanized | | | |
| | | construct H6 | | | |
| MUS276 | NOGO | Heavy chain | 2A10 construct | WO2007003421 | 5922 |
| | | variable region | | SEQ ID NO: 13 | |
| | | humanized | | | |
| | | construct H700 | | | |
| MUS277 | ACVR2B | Light chain | H7L4, H7L5, H7L6 | U.S. Pat. No. | 5923 |
| | (SMA - muscle | | | 8,388,968 | |
| | growth) | | | SEQ ID NO: 141 | |
| MUS278 | ACVR2B | Light chain | | U.S. Pat. No. | 5924 |
| | | | | 8,388,968 | |
| | | | | SEQ ID NO: 141 | |
| MUS279 | amyloids | Light chain | #118 | WO2010012004 | 5925 |
| | | | | SEQ ID NO: 10 | |
| MUS280 | amyloids | Light chain | #121 | WO2010012004 | 5926 |
| | | | | SEQ ID NO: 12 | |
| MUS281 | amyloids | Light chain | #201 | WO2010012004 | 5927 |
| | | | | SEQ ID NO: 14 | |
| MUS282 | amyloids | Light chain | #204 | WO2010012004 | 5928 |
| | | | | SEQ ID NO: 15 | |
| MUS283 | amyloids | Light chain | #205 | WO2010012004 | 5929 |
| | | | | SEQ ID NO: 17 | |
| MUS284 | EAG1 | Light chain | chimeric ImAb3 | WO2006037604 | 5930 |
| | | | | SEQ ID NO: 10 | |
| MUS285 | EAG1 | Light chain | chimeric ImAb4 | WO2006037604 | 5931 |
| | | | | SEQ ID NO: 14 | |
| MUS286 | EAG1 | Light chain | LC-ImAb3-humB3 | WO2006037604 | 5932 |
| | | | | SEQ ID NO: 18 | |
| MUS287 | EAG1 | Light chain | ImAb4 | WO2006037604 | 5933 |
| | | | | SEQ ID NO: 2 | |
| MUS288 | EAG1 | Light chain | LC-lmAb4-humA17 | WO2006037604 | 5934 |
| | | | | SEQ ID NO: 22 | |
| MUS289 | EAG1 | Light chain | LC-lmAb3-humA3 | WO2006037604 | 5935 |
| | | | | SEQ ID NO: 26 | |
| MUS290 | EAG1 | Light chain | LC-lmAb3-humA17 | WO2006037604 | 5936 |
| | | | | SEQ ID NO: 30 | |
| MUS291 | EAG1 | Light chain | LC-lmAb4-humA5-1 | WO2006037604 | 5937 |
| | | | | SEQ ID NO: 34 | |
| MUS292 | EAG1 | Light chain | LC-lmAb4-humO1 | WO2006037604 | 5938 |
| | | | | SEQ ID NO: 38 | |
| MUS293 | EAG1 | Light chain | ImAb3 | WO2006037604 | 5939 |
| | | | | SEQ ID NO: 6 | |
| MUS294 | GDF-8 | Light chain | 597-120-M1 | US20130287762 | 5940 |
| | | | | SEQ ID NO: 12 | |
| MUS295 | GDF-8 | Light chain | 597-120 | US20130287762 | 5941 |
| | | | | SEQ ID NO: 18 | |
| MUS296 | GDF-8 | Light chain | 311-3 | US20130287762 | 5942 |
| | | | | SEQ ID NO: 6 | |
| MUS297 | growth | Light chain | | | 5943 |
| | differentiation | | | | |
| | factor 8 | | | | |
| MUS298 | growth | Light chain | Domagrozumab | | 5944 |
| | differentiation | | | | |
| | factor 8 | | | | |
| MUS299 | MAG | Light chain | | | 5945 |
| MUS300 | MSTN | Light chain | H25L13, H25L16, | | 5946 |
| | | | H25L18, H25L14, | | |
| | | | H25L15, H25L17, | | |
| | | | H25L6, H25L11 | | |
| MUS301 | Myostatin | Light chain | 306-155 | US20130142788 | 5947 |
| | | | | SEQ ID NO: 145 | |
| MUS302 | Myostatin | Light chain | 14-173 | US20130142788 | 5948 |
| | | | | SEQ ID NO: 146 | |
| MUS303 | Myostatin | Light chain | 14-173-M1 | US20130142788 | 5949 |
| | | | | SEQ ID NO: 147 | |
| MUS304 | myostatin | Light chain | NI-204.67E12 | US20110256132 | 5950 |
| | | | | SEQ ID NO: 27 | |
| MUS305 | myostatin | Light chain | NI-204.12G3 | US20110256132 | 5951 |
| | | | | SEQ ID NO: 31 | |
| MUS306 | myostatin | Light chain | NI-204.12G3 | US20110256132 | 5952 |
| | | | | SEQ ID NO: 32 | |
| MUS307 | myostatin | Light chain | NI-204.7G5 | US20110256132 | 5953 |
| | | | | SEQ ID NO: 33 | |
| MUS308 | myostatin | Light chain | NI-204.7G5 | US20110256132 | 5954 |
| | | | | SEQ ID NO: 34 | |
| MUS309 | Myostatin | Light chain | 114-41-M1 | US20130142788 | 5955 |
| | | | | SEQ ID NO: 27 | |
| MUS310 | Myostatin | Light chain | 14-173, 14-173-M1 | US20130142788 | 5956 |
| | | | | SEQ ID NO: 33 | |
| MUS311 | Myostatin | Light chain | 306-155 | US20130142788 | 5957 |
| | | | | SEQ ID NO: 37 | |
| MUS312 | Myostatin | Light chain | 303-8 | US20130142788 | 5958 |
| | | | | SEQ ID NO: 40 | |
| MUS313 | myostatin | Light chain | N1-204.34A3 | US20130209489 | 5959 |
| | antagonists | | | SEQ ID NO: 1 | |
| MUS314 | myostatin | Light chain | NI-205.21G2 | US20130209489 | 5960 |
| | antagonists | | | SEQ ID NO: 10 | |
| MUS315 | myostatin | Light chain | NI-204.25H3 | US20130209489 | 5961 |
| | antagonists | | | SEQ ID NO: 2 | |
| MUS316 | myostatin | Light chain | NI-204.25H3 | US20130209489 | 5962 |
| | antagonists | | | SEQ ID NO: 3 | |
| MUS317 | myostatin | Light chain | B12 | US20130209489 | 5963 |
| | antagonists | | | SEQ ID NO: 4 | |
| MUS318 | myostatin | Light chain | B1 | US20130209489 | 5964 |
| | antagonists | | | SEQ ID NO: 5 | |
| MUS319 | myostatin | Light chain | NI-205.3F10 | US20130209489 | 5965 |
| | antagonists | | | SEQ ID NO: 6 | |
| MUS320 | myostatin | Light chain | NI-205.3F10 | US20130209489 | 5966 |
| | antagonists | | | SEQ ID NO: 7 | |
| MUS321 | myostatin | Light chain | NI-205.51C1 | US20130209489 | 5967 |
| | antagonists | | | SEQ ID NO: 8 | |
| MUS322 | myostatin | Light chain | NI-205.51C1 | US20130209489 | 5968 |
| | antagonists | | | SEQ ID NO: 9 | |
| MUS323 | NOGO | Light chain | H6L13 FL, H19L13 | US20140147435 | 5969 |
| | | | FL, H20L 13 FL, | SEQ ID NO: 35 | |
| | | | H21L13 FL, H25L13 | | |
| | | | FL | | |
| MUS324 | NOGO | Light chain | H16L16 FL, H19L16 | US20140147435 | 5970 |
| | | | FL, H20L16 FL, | SEQ ID NO: 38 | |
| | | | H21L16 FL, H25L16 | | |
| | | | FL, H18L16 FL | | |
| MUS325 | NOGO | Light chain | H16L18 FL, H19L18 | US20140147435 | 5971 |
| | | | FL, H20L18 FL, | SEQ ID NO: 40 | |
| | | | H21L18 FL, H25L18 | | |
| | | | FL | | |
| MUS326 | Nogo receptor- | Light chain | 7E11 | US20090215691 | 5972 |
| | 1 | | | SEQ ID NO: 15 | |
| MUS327 | Nogo receptor- | Light chain | 7E11 | US20090215691 | 5973 |
| | 1 | | | SEQ ID NO: 17 | |
| MUS328 | RTN4 | Light chain | | | 5974 |
| MUS329 | SIP4 | Light chain | | WO2015057939 | 5975 |
| | | | | SEQ ID NO: 41 | |
| MUS330 | trk-C (NT-3 | Light chain | 2250 | U.S. Pat. No. | 5976 |
| | trkC ligand) | | | 7,615,383 | |
| | | | | SEQ ID NO: 49 | |
| MUS331 | trk-C (NT-3 | Light chain | 2253 | U.S. Pat. No. | 5977 |
| | trkC ligand) | | | 7,615,383 | |
| | | | | SEQ ID NO: 50 | |
| MUS332 | trk-C (NT-3 | Light chain | 2256 | U.S. Pat. No. | 5978 |
| | trkC ligand) | | | 7,615,383 | |
| | | | | SEQ ID NO: 51 | |
| MUS333 | trkc-C (NT-3 | Light chain | 6.1.2 | U.S. Pat. No. | 5979 |
| | trkC ligand) | | | 7,615,383 | |
| | | | | SEQ ID NO: 52 | |
| MUS334 | trk-C (NT-3 | Light chain | 6.4.1 | U.S. Pat. No. | 5980 |
| | trkC ligand) | | | 7,615,383 | |
| | | | | SEQ ID NO: 53 | |
| MUS335 | trk-C (NT-3 | Light chain | 2345 | U.S. Pat. No. | 5981 |
| | turkC ligand) | | | 7,615,383 | |
| | | | | SEQ ID NO: 54 | |
| MUS336 | trk-C (NT-3 | Light chain | 2349 | U.S. Pat. No. | 5982 |
| | trkC ligand) | | | 7,615,383 | |
| | | | | SEQ ID NO: 55 | |
| MUS337 | GDF-8 | Light chain | 12A5-18HC | U.S. Pat. No. | 5983 |
| | | constant region | | 8,956,608 | |
| | | | | SEQ ID NO: 17 | |
| MUS338 | many - growth | Light chain fusion | H21L.13, H21L16, | U.S. Pat. No. | 5984 |
| | factors (to | protein | H21L18, H21L14, | 8,053,569 | |
| | increase | | H21L15, H21L17, | SEQ ID NO: 31 | |
| | transport across | | H21L6, H21L11 | | |
| | BBB) | | | | |
| MUS339 | many - growth | Light chain fusion | H23L13, H23L16, | U.S. Pat. No. | 5985 |
| | factors (to | protein | H23L.18, H23L14, | 8,053,569 | |
| | increase | | H23L15, H23L17, | SEQ ID NO: 36 | |
| | transport across | | H23L6, H23L11 | | |
| | BBB) | | | | |
| MUS340 | NOGO | Light chain | 2A10 construct | WO2007003421 | 5986 |
| | | humanized | | SEQ ID NO: 80 | |
| | | construct L11 | | | |
| MUS341 | NOGO | Light chain | 2A10 construct | WO2007003421 | 5987 |
| | | humanized | | SEQ ID NO: 35 | |
| | | construct L 13 | | | |
| MUS342 | NOGO | Light chain | 2A10 construct | WO2007003421 | 5988 |
| | | humanized | | SEQ ID NO: 36 | |
| | | construct L14 | | | |
| MUS343 | NOGO | Light chain | 2A10 construct | WO2007003421 | 5989 |
| | | humanized | | SEQ ID NO: 37 | |
| | | construct L15 | | | |
| MUS344 | NOGO | Light chain | 2.A10 construct | WO2007003421 | 5990 |
| | | humanized | | SEQ ID NO: 38 | |
| | | construct L 16 | | | |
| MUS345 | NOGO | Light chain | 2A10 construct | WO2007003421 | 5991 |
| | | humanized | | SEQ ID NO: 39 | |
| | | construct L17 | | | |
| MUS346 | NOGO | Light chain | 2A10 construct | WO2007003421 | 5992 |
| | | humanized | | SEQ ID NO: 40 | |
| | | construct L18 | | | |
| MUS347 | NOGO | Light chain | 2A10 construct | WO2007003421 | 5993 |
| | | humanized | | SEQ ID NO: 34 | |
| | | construct L6 | | | |
| MUS348 | RTN4 | Light chain IgG4, | Atinumab | U.S. Pat. No. | 5994 |
| | | immunomodulator | | 8,163,285 | |
| | | | | SEQ ID NO: 25 | |
| MUS349 | amyloid | Light chain | F11G3 | U.S. Pat. No. | 5995 |
| | oligomers | variable region | | 9,125,846 | |
| | | | | SEQ ID NO: 12 | |
| MUS350 | differentiation | Light chain | H9L4, H9L5, H8L6 | US20140023638 | 5996 |
| | factor 8 | variable region | | SEQ ID NO: 18 | |
| | (GDF8) | | | | |
| MUS351 | DR6 and P75 | Light chain | M73-C04 | WO2010062904 | 5997 |
| | | variable region | | SEQ ID NO: 102 | |
| MUS352 | DR6 and P75 | Light chain | 1PID6.3 | WO2010062904 | 5998 |
| | | variable region | | SEQ ID NO: 112 | |
| MUS353 | DR6 and P75 | Light chain | M50-H02 | WO2010062904 | 5999 |
| | | variable region | | SEQ ID NO: 12 | |
| MUS354 | DR6 and P75 | Light chain | 1P2F2.1 | WO2010062904 | 6000 |
| | | variable region | | SEQ ID NO: 122 | |
| MUS355 | DR6 and P75 | Light chain | 1P5D10.2 | WO2010062904 | 6001 |
| | | variable region | | SEQ ID NO: 132 | |
| MUS356 | DR6 and P75 | Light chain | M51-H09 | WO2010062904 | 6002 |
| | | variable region | | SEQ ID NO: 22 | |
| MUS357 | DR6 and P75 | Light chain | M53-E04 | WO2010062904 | 6003 |
| | | variable region | | SEQ ID NO: 32 | |
| MUS358 | DR6 and P75 | Light chain | M53-F04 | WO2010062904 | 6004 |
| | | variable region | | SEQ ID NO: 42 | |
| MUS359 | DR6 and P75 | Light chain | M62-B02 | WO2010062904 | 6005 |
| | | variable region | | SEQ ID NO: 52 | |
| MUS360 | DR6 and P75 | Light chain | M63-E10 | WO2010062904 | 6006 |
| | | variable region | | SEQ ID NO: 62 | |
| MUS361 | DR6 and P75 | Light chain | M66-B03 | WO2010062904 | 6007 |
| | | variable region | | SEQ ID NO: 72 | |
| MUS362 | DR6 and P75 | Light chain | M67-G02 | WO2010062904 | 6008 |
| | | variable region | | SEQ ID NO: 82 | |
| MUS363 | DR6 and P75 | Light chain | M72-F03 | WO2010062904 | 6009 |
| | | variable region | | SEQ ID NO: 92 | |
| MUS364 | GDF-8 | Light chain | 595-16- M1 | U.S. Pat. No. | 6010 |
| | | variable region | | 8,956,608 | |
| | | | | SEQ ID NO: 27 | |
| MUS365 | GDF-8 | Light chain | A12A5-12HC | U.S. Pat. No. | 6011 |
| | | variable region | | 8,956,608 | |
| | | | | SEQ ID NO: 9 | |
| MUS366 | growth | Light chain | | U.S. Pat. No. | 6012 |
| | differentiation | variable region | | 8,840,894 | |
| | factor 8 | | | SEQ ID NO: 368 | |
| MUS367 | LPG | Light chain | #7 | U.S. Pat. No. | 6013 |
| | (lysophosphatid | variable region | | 8,591,902 | |
| | ylglucoside) | | | SEQ ID NO: 17 | |
| MUS368 | LPG | Light chain | #15 | U.S. Pat. No. | 6014 |
| | (lysophosphatid | variable region | | 8,591,902 | |
| | ylglucoside) | | | SEQ ID NO: 7 | |
| MUS369 | MAG | Light chain | | U.S. Pat. No. | 6015 |
| | | variable region | | 8,071,731 | |
| | | | | SEQ ID NO: 16 | |
| MUS370 | MAG | Light chain | | U.S. Pat. No. | 6016 |
| | | variable region | | 8,071,731 | |
| | | | | SEQ ID NO: 17 | |
| MUS371 | MAG | Light chain | | U.S. Pat. No. | 6017 |
| | | variable region | | 8,071,731 | |
| | | | | SEQ ID NO: 18 | |
| MUS372 | MAG | Light chain | | U.S. Pat. No. | 6018 |
| | | variable region | | 8,071,731 | |
| | | | | SEQ ID NO: 19 | |
| MUS373 | MAI (myelin | Light chain | | WO2013158748 | 6019 |
| | associated | variable region | | SEQ ID NO: 11 | |
| | inhibitor) | | | | |
| MUS374 | MAI (myelin | Light chain | | WO2013158748 | 6020 |
| | associated | variable region | | SEQ ID NO: 27 | |
| | inhibitor) | | | | |
| MUS375 | myostatin | Light chain | NI-204.34A3 | SEQ ID 8 WO | 6021 |
| | | variable region | | 2006107611 | |
| MUS376 | myostatin | Light chain | NI-204.9F6 | US20110256132 | 6022 |
| | | variable region | | SEQ ID NO: 17 | |
| MUS377 | myostatin | Light chain | NI-204.9F6 | US20110256132 | 6023 |
| | | variable region | | SEQ ID NO: 21 | |
| MUS378 | myostatin | Light chain | NI-204.11F11 | US20110256132 | 6024 |
| | | variable region | | SEQ ID NO: 24 | |
| MUS379 | myostatin | Light chain | 303-8 | US20110256132 | 6025 |
| | | variable region | | SEQ ID NO: 7 | |
| MUS380 | NOGO | Light chain | H1L6, H5L6, H6L6, | US20140147435 | 6026 |
| | | variable region | H14L6, H15L6, | SEQ ID NO: 19 | |
| | | | H16L6, H17L6, | | |
| | | | H18L6, H19L6, | | |
| | | | H20L6, H21L6, | | |
| | | | H22L6, H23L6, | | |
| | | | H24L6, H25L6, | | |
| | | | H700L6 | | |
| MUS381 | NOGO | Light chain | H1L13, H5L13, | US20140147435 | 6027 |
| | | variable region | H6L13, H14L13, | SEQ ID NO: 20 | |
| | | | H15L13, H16L13, | | |
| | | | H17L.13, H18L.13, | | |
| | | | H19L13, H20L.13, | | |
| | | | H21L13, H22L.13, | | |
| | | | H23L13, H24L13, | | |
| | | | H25L13, H700L13 | | |
| MUS382 | NOGO | Light chain | H1L 14, H5L14, | US20140147435 | 6028 |
| | | variable region | H6L 14, H14L14, | SEQ ID NO: 21 | |
| | | | H15L.14, H16L14, | | |
| | | | H17L.14, H18L14, | | |
| | | | H19L14, H20L14, | | |
| | | | H21L14, H22L14, | | |
| | | | H23L.14, H24L14, | | |
| | | | H25L14, H700L14 | | |
| MUS383 | NOGO | Light chain | H1L15, H5L15, | US20140147435 | 6029 |
| | | variable region | H6L15. H14L15, | SEQ ID NO: 22 | |
| | | | H15L15, H16L15, | | |
| | | | H17L15, H18L15, | | |
| | | | H19L15, H20L15, | | |
| | | | H21L15, H22L15, | | |
| | | | H23L15, H24L15, | | |
| | | | H25L15, H700L15 | | |
| MUS384 | NOGO | Light chain | H1L16, H5L16, | US20140147435 | 6030 |
| | | variable region | H6L16, H14L16 | SEQ ID NO: 23 | |
| | | | H15L16, H16L16, | | |
| | | | H17L16, H18L16, | | |
| | | | H19L16, H20L16, | | |
| | | | H21L16, H22L16, | | |
| | | | H23L16, H24L16, | | |
| | | | H25L16, H700L16 | | |
| MUS385 | NOGO | Light chain | H1L. 17, HSL17, | US20140147435 | 6031 |
| | | variable region | H6L.17, H14L.17, | SEQ ID NO: 24 | |
| | | | H15L17, H16L17, | | |
| | | | H17L17, H18L17, | | |
| | | | H19L17, H20L17, | | |
| | | | H21L17, H22L17, | | |
| | | | H23L17, H24L17, | | |
| | | | H25L17, H700L.17 | | |
| MUS386 | NOGO | Light chain | H1L18, H5L18, | US20140147435 | 6032 |
| | | variable region | H6L 18, H14L18, | SEQ ID NO: 25 | |
| | | | H15L18, H16L18, | | |
| | | | H17L18, H18L18, | | |
| | | | H19L18, H20L18, | | |
| | | | H21L18, H22L18, | | |
| | | | H23L18, H24L18, | | |
| | | | H25L18. H700L18 | | |
| MUS387 | NOGO | Light chain | H5L11, H6L11, | US20140147435 | 6033 |
| | | variable region | H14L11, H15L11, | SEQ ID NO: 78 | |
| | | | H16L11, H17L11, | | |
| | | | H18L11, H19L11, | | |
| | | | H20L11, H21L11, | | |
| | | | H22L11, H23L11, | | |
| | | | H24L11, H25L11, | | |
| | | | H700L11 | | |
| MUS388 | Nogo-66 | Light chain | Antibody clone 50 | US20140065155 | 6034 |
| | | variable region | | SEQ ID NO: 4 | |
| MUS389 | Nogo-66 | Light chain | Antibody clone 51 | US20140065155 | 6035 |
| | | variable region | | SEQ ID NO: 6 | |
| MUS390 | NogoA/NIG | Light chain | 6A3-Ig4 | WO2009056509 | 6036 |
| | | variable region | | SEQ ID NO: 25 | |
| MUS391 | NogoA/NIG | Light chain | 6A3-IgGI | WO2009056509 | 6037 |
| | | variable region | | SEQ ID NO: 5 | |
| MUS392 | RGM A | Light chain | 5F9.1-GL, 5F9.1-GL, | US20150183871 | 6038 |
| | | variable region | 5F9.1-GL, SF9.1-GL, | SEQ ID NO: 44 | |
| | | | 5F9.1-GL, 5F9.1-GL, | | |
| | | | 5F9.1-GL, 5F9.1-GL, | | |
| | | | 5F9.1-GL, 5F9.1-GL, | | |
| | | | h5F9.4, h5F9.11, | | |
| | | | h5F9.12 | | |
| MUS393 | RGM A | Light chain | 5F9.2-GL, 5F9.2-GL, | US20150183871 | 6039 |
| | | variable region | 5F9.2-GL, 5F9.2-GL, | SEQ ID NO: 45 | |
| | | | 5F9.2-GL, 5F9.2-GL, | | |
| | | | 5F9.2-GL, 5F9.2-GL, | | |
| | | | 5F9.2-GL, 5F9.2-GL, | | |
| | | | h5F9.5, h5F9.19, | | |
| | | | h5F9.20 | | |
| MUS394 | RGM A | Light chain | 5F9.3-GL, 5F9.3-GL, | US20150183871 | 6040 |
| | | variable region | 5F9.3-GL, 5F9.3-GL, | SEQ ID NO: 46 | |
| | | | 5F9.3-GL, 5F9.3-GL, | | |
| | | | 5F9.3-GL, 5F9.3-GL, | | |
| | | | 5F9.3-GL, 5F9.3-GL, | | |
| | | | h5F9.6, h5F9.21, | | |
| | | | h5F9.22 | | |
| MUS395 | RGM A | Light chain | h5F9.5, h5F9.6, | US20150183871 | 6041 |
| | | variable region | hSF9.7, h5F9.8, | SEQ ID NO: 48 | |
| | | | h5F9.9. h5F9.10 | | |
| MUS396 | RGM A | Light chain | h5F9.11, | US20150183871 | 6042 |
| | | variable region | h5F9.19, h5F9.21 | SEQ ID NO: 49 | |
| MUS397 | RGM A | Light chain | h5F9.12, h5F9.20, | US20150183871 | 6043 |
| | | variable region | h5F9.22, h5F9.23, | SEQ ID NO: 50 | |
| | | | h5F9.25, h5F9.25, | | |
| | | | h5F9.26 | | |
| MUS398 | RGM A | Light chain | h5F9.1, h5F9.7, | US20150183871 | 6044 |
| | | variable region | h5F9.23 | SEQ ID NO: 51 | |
| MUS399 | RGM A | Light chain | hSF9.2, h5F9.8, | US20150183871 | 6045 |
| | | variable region | h5F9.25 | SEQ ID NO: 52 | |
| MUS400 | RGMa | Light chain | AE12-15 | US20140023659 | 6046 |
| | | variable region | | SEQ ID NO: 103 | |
| MUS401 | RGMa | Light chain | AE12-20 | US20140023659 | 6047 |
| | | variable region | | SEQ ID NO: 111 | |
| MUS402 | RGMa | Light chain | AE12-21 | US20140023659 | 6048 |
| | | variable region | | SEQ ID NO: 119 | |
| MUS403 | RGMa | Light chain | AE12-23 | US20140023659 | 6049 |
| | | variable region | | SEQ ID NO: 127 | |
| MUS404 | RGMa | Light chain | AE12-2 | US20140023659 | 6050 |
| | | variable region | | SEQ ID NO: 13 | |
| MUS405 | RGMa | Light chain | AE12-24 | US20140023659 | 6051 |
| | | variable region | | SEQ ID NO: 135 | |
| MUS406 | RGMa | Light chain | AE12-3 | US20140023659 | 6052 |
| | | variable region | | SEQ ID NO: 21 | |
| MUS407 | RGMa | Light chain | AE12-4 | US20140023659 | 6053 |
| | | variable region | | SEQ ID NO: 29 | |
| MUS408 | RGMa | Light chain | AE12-5 | US20140023659 | 6054 |
| | | variable region | | SEQ ID NO: 37 | |
| MUS409 | RGMa | Light chain | AE12-6 | US20140023659 | 6055 |
| | | variable region | | SEQ ID NO: 45 | |
| MUS410 | RGMa | Light chain | AE12-1 | US20140023659 | 6056 |
| | | variable region | | SEQ ID NO: 5 | |
| MUS411 | RGMa | Light chain | AE12-7 | US20140023659 | 6057 |
| | | variable region | | SEQ ID NO: 53 | |
| MUS412 | RGMa | Light chain | AE12-8 | US20140023659 | 6058 |
| | | variable region | | SEQ ID NO: 61 | |
| MUS413 | RGMa | Light chain | AE12-13 | US20140023659 | 6059 |
| | | variable region | | SEQ ID NO: 95 | |
| MUS414 | SIP4 | Light chain | | WO2015057939 | 6060 |
| | | variable region | | SEQ ID NO: 9 | |
| MUS415 | SOD1 | Light chain | NI205.14W3 | US20140301945 | 6061 |
| | | variable region | | SEQ ID NO: 10 | |
| MUS416 | SODI | Light chain | NI205.19G5 | US20140301945 | 6062 |
| | | variable region | | SEQ ID NO: 14 | |
| MUS417 | SOD1 | Light chain | | US20140301945 | 6063 |
| | | variable region | | SEQ ID NO: 18 | |
| MUS418 | SOD1 | Light chain | | US20140301945 | 6064 |
| | | variable region | | SEQ ID NO: 22 | |
| MUS419 | SOD1 | Light chain | | US20140301945 | 6065 |
| | | variable region | | SEQ ID NO: 26 | |
| MUS420 | SOD1 | Light chain | Landogrozumab, | US20140301945 | 6066 |
| | | variable region | LY2495655, LY- | SEQ ID NO: 30 | |
| | | | 2495656 | | |
| MUS421 | SOD1 | Light chain | 2A10 construct | US20140301945 | 6067 |
| | | variable region | | SEQ ID NO: 34 | |
| MUS422 | SOD1 | Light chain | 2A10 construct | US20140301945 | 6068 |
| | | variable region | | SEQ ID NO: 38 | |
| MUS423 | SOD1 | Light chain | 2A10 construct | US20140301945 | 6069 |
| | | variable region | | SEQ ID NO: 42 | |
| MUS424 | SOD1 | Light chain | 2A10 construct | US20140301945 | 6070 |
| | | variable region | | SEQ ID NO: 46 | |
| MUS425 | SODI | Light chain | 2A10 construct | US20140301945 | 6071 |
| | | variable region | | SEQ ID NO: 50 | |
| MUS426 | SOD1 | Light chain | NI205.1A9 | US20140301945 | 6072 |
| | | variable region | | SEQ ID NO: 6 | |
| MUS427 | SOD1 | Light chain | NI205.31D2 | US9109037 SEQ | 6073 |
| | | variable region | | ID NO: 109 | |
| MUS428 | SODI | Light chain | NI205.8F8 | US9109037 SEQ | 6074 |
| | | variable region | | ID NO: 121 | |
| MUS429 | SODI | Light chain | NI205.8F8 | US9109037 SEQ | 6075 |
| | | variable region | | ID NO: 139 | |
| MUS430 | SOD1 | Light chain | NI205.31C11 | US9109037 SEQ | 6076 |
| | | variable region | | ID NO: 157 | |
| MUS431 | SOD1 | Light chain | NI205.31C11 | US9109037 SEQ | 6077 |
| | | variable region | | ID NO: 175 | |
| MUS432 | SODI | Light chain | NI205.8C10 | US9109037 SEQ | 6078 |
| | | variable region | | ID NO: 191 | |
| MUS433 | SODI | Light chain | NI205,8C10 | US9109037 SEQ | 6079 |
| | | variable region | | ID NO: 199 | |
| MUS434 | SOD1 | Light chain | NI205.10H7 | US9109037 SEQ | 6080 |
| | | variable region | | ID NO: 209 | |
| MUS435 | SOD1 | Light chain | NI205.10H7 | US9109037 SEQ | 6081 |
| | | variable region | | ID NO: 225 | |
| MUS436 | SODI | Light chain | NI205.58E11 | US9109037 SEQ | 6082 |
| | | variable region | | ID NO: 27 | |
| MUS437 | SOD1 | Light chain | NI205.58E11 | US9109037 SEQ | 6083 |
| | | variable region | | ID NO: 45 | |
| MUS438 | SOD1 | Light chain | NI205.14H5 | US9109037 SEQ | 6084 |
| | | variable region | | ID NO: 63 | |
| MUS439 | SOD1 | Light chain | NI205.14H5 | US9109037 SEQ | 6085 |
| | | variable region | | ID NO: 81 | |
| MUS440 | SOD1 | Light chain | NI205.36D5 | US9109037 SEQ | 6086 |
| | | variable region | | ID NO: 9 | |
| MUS441 | SOD1 | Light chain | NI205.31D2 | US9109037 SEQ | 6087 |
| | | variable region | | ID NO: 99 | |
| MUS442 | TDP-43 | Light chain | 2A10 construct | US20140255304 | 6088 |
| | | variable region | | SEQ ID NO: 122 | |
| MUS443 | TDP-43 | Light chain | 2A10 construct | US20140255304 | 6089 |
| | | variable region | | SEQ ID NO: 134 | |
| MUS444 | TDP-43 | Light chain | 2A10 construct | US20140255304 | 6090 |
| | | variable region | | SEQ ID NO: 14 | |
| MUS445 | TDP-43 | Light chain | 2A10 construct | US20140255304 | 6091 |
| | | variable region | | SEQ ID NO: 142 | |
| MUS446 | TDP-43 | Light chain | 2A10 construct | US20140255304 | 6092 |
| | | variable region | | SEQ ID NO: 150 | |
| MUS447 | TDP-43 | Light chain | 2A10 construct | US20140255304 | 6093 |
| | | variable region | | SEQ ID NO: 155 | |
| MUS448 | TDP-43 | Light chain | 2A10 construct | US20140255304 | 6094 |
| | | variable region | | SEQ ID NO: 163 | |
| MUS449 | TDP-43 | Light chain | 2A10 construct | US20140255304 | 6095 |
| | | variable region | | SEQ ID NO: 171 | |
| MUS450 | TDP-43 | Light chain | 2A10 construct | US20140255304 | 6096 |
| | | variable region | | SEQ ID NO: 179 | |
| MUS451 | TDP-43 | Light chain | H16L16 FL, H19L16 | US20140255304 | 6097 |
| | | variable region | FL, H20L16 FL, | SEQ ID NO: 187 | |
| | | | H21L16 FL, H25L16 | | |
| | | | FL, H18L16 FL | | |
| MUS452 | TDP-43 | Light chain | H6L13 FL | US20140255304 | 6098 |
| | | variable region | | SEQ ID NO: 195 | |
| MUS453 | TDP-43 | Light chain | H5L13, H5L16, | US20140255304 | 6099 |
| | | variable region | H5L18, H5L14, | SEQ ID NO: 203 | |
| | | | H5L15, H5L17, H5L6, | | |
| | | | H5L11 | | |
| MUS454 | TDP-43 | Light chain | H700L13, H700L16, | US20140255304 | 6100 |
| | | variable region | H700L18, H700L14, | SEQ ID NO: 211 | |
| | | | H700L15, H700L17, | | |
| | | | H700L6, H700L11 | | |
| MUS455 | TDP-43 | Light chain | H15L13, H15L16, | US20140255304 | 6101 |
| | | variable region | H15L18, H15L14, | SEQ ID NO: 219 | |
| | | | H15L15, H15L17, | | |
| | | | H15L6, H15L11 | | |
| MUS456 | TDP-43 | Light chain | 2A10 construct | US20140255304 | 6102 |
| | | variable region | | SEQ ID NO: 22 | |
| MUS457 | TDP-43 | Light chain | H17L13, H17L16, | US20140255304 | 6103 |
| | | variable region | H17L18, H17L14, | SEQ ID NO: 227 | |
| | | | H17L15, H17L17, | | |
| | | | H17L6. H17L11 | | |
| MUS458 | TDP-43 | Light chain | H1L6, H5L6, H6L6, | US20140255304 | 6104 |
| | | variable region | H14L6, H15L6, | SEQ ID NO: 235 | |
| | | | H16L6, H17L6, | | |
| | | | H18L6, H19L6, | | |
| | | | H20L6, H21L6, | | |
| | | | H22L6, H23L6, | | |
| | | | H24L6, H25L6, | | |
| | | | H700L6 | | |
| MUS459 | TDP-43 | Light chain | H1L14, H5L14, | US20140255304 | 6105 |
| | | variable region | H6114, H14L14, | SEQ ID NO: 243 | |
| | | | H15L14, H16L14, | | |
| | | | H17L14, H18L14, | | |
| | | | H19L14, H20L14, | | |
| | | | H21L14, H22L14, | | |
| | | | H23L14, H24L14, | | |
| | | | H25L14, H700114 | | |
| MUS460 | TDP-43 | Light chain | H1L16, H5L16, | US20140255304 | 6106 |
| | | variable region | H6L16, H14L16, | SEQ ID NO: 251 | |
| | | | H15L16, H16L16, | | |
| | | | H17L16, H18L16, | | |
| | | | H19L16, H20L16, | | |
| | | | H21L16, H22L16, | | |
| | | | H23L16, H24L16, | | |
| | | | H25L16, H700L16 | | |
| MUS461 | TDP-43 | Light chain | H1L18, H5L18, | US20140255304 | 6107 |
| | | variable region | H6L18. H14L18, | SEQ ID NO: 259 | |
| | | | H15L18, H16L18, | | |
| | | | H17L18, H18L18, | | |
| | | | H19L18, H20L18, | | |
| | | | H21L18, H22L18, | | |
| | | | H23L18, H24L18, | | |
| | | | H25L18, H700L18 | | |
| MUS462 | TDP-43 | Light chain | H1L13, H1L16, | US20140255304 | 6108 |
| | | variable region | H1L18, H1L14, | SEQ ID NO: 267 | |
| | | | H1L15, H1L17, H1L6 | | |
| MUS463 | TDP-43 | Light chain | 2A10 construct | US20140255304 | 6109 |
| | | variable region | | SEQ ID NO: 31 | |
| MUS464 | TDP-43 | Light chain | 2A10 construct | US20140255304 | 6110 |
| | | variable region | | SEQ ID NO: 40 | |
| MUS465 | TDP-43 | Light chain | 2.A10 construct | US20140255304 | 6111 |
| | | variable region | | SEQ ID NO: 49 | |
| MUS466 | TDP-43 | Light chain | 2A10 construct | US20140255304 | 6112 |
| | | variable region | | SEQ ID NO: 57 | |
| MUS467 | TDP-43 | Light chain | 2A10 construct | US20140255304 | 6113 |
| | | variable region | | SEQ ID NO: 6 | |
| MUS468 | TDP-43 | Light chain | 2A10 construct | US20140255304 | 6114 |
| | | variable region | | SEQ ID NO: 65 | |
| MUS469 | TDP-43 | Light chain | 2A10 construct | US20140255304 | 6115 |
| | | variable region | | SEQ ID NO: 73 | |
| MUS470 | TDP-43 | Light chain | 2A10 construct | US20140255304 | 6116 |
| | | variable region | | SEQ ID NO: 82 | |
| MUS471 | trkC | Light chain | | US20070031418 | 6117 |
| | | variable region | | SEQ ID NO: 2 | |
| MUS472 | NOGO | Light chain | 2A10 construct | WO2007003421 | 6118 |
| | | variable region | | SEQ ID NO: 78 | |
| | | humanized | | | |
| | | construct L11 | | | |
| MUS473 | NOGO | Light chain | 2A10 construct | WO2007003421 | 6119 |
| | | variable region | | SEQ ID NO: 20 | |
| | | humanized | | | |
| | | construct L.13 | | | |
| MUS474 | NOGO | Light chain | 2A10 construct | WO2007003421 | 6120 |
| | | variable region | | SEQ ID NO: 21 | |
| | | humanized | | | |
| | | construct L14 | | | |
| MUS475 | NOGO | Light chain | 2A10 construct | WO2007003421 | 6121 |
| | | variable region | | SEQ ID NO: 22 | |
| | | humanized | | | |
| | | construct L15 | | | |
| MUS476 | NOGO | Light chain | 2A10 construct | WO2007003421 | 6122 |
| | | variable region | | SEQ ID NO: 23 | |
| | | humanized | | | |
| | | construct L16 | | | |
| MUS477 | NOGO | Light chain | 2A10 construct | WO2007003421 | 6123 |
| | | variable region | | SEQ ID NO: 24 | |
| | | humanized | | | |
| | | construct L17 | | | |
| MUS478 | NOGO | Light chain | 2A10 construct | WO2007003421 | 6124 |
| | | variable region | | SEQ ID NO: 25 | |
| | | humanized | | | |
| | | construct L18 | | | |
| MUS479 | NOGO | Light chain | 2A10 construct | WO2007003421 | 6125 |
| | | variable region | | SEQ ID NO: 19 | |
| | | humanized | | | |
| | | construct L6 | | | |
| MUS480 | GDF-8 | scFy | 591-37 | US20130287762 | 6126 |
| | | | | SEQ ID NO: 14 | |
| MUS481 | GDF-8 | scFv | 358-11 | US20130287762 | 6127 |
| | | | | SEQ ID NO: 2 | |
| MUS482 | GDF-8 | scFv | 591-37-MI | US20130287762 | 6128 |
| | | | | SEQ ID NO: 8 | |
| MUS483 | myostatin | scFv | NI-204.7B3 | SEQ ID 4 WO | 6129 |
| | | | | 2006107611 | |
| MUS484 | SOD1 | scFv | 2A10 construct | Ghadge, G.D. et | 6130 |
| | | | | al., Single chain | |
| | | | | variable fragment | |
| | | | | antibodies block | |
| | | | | aggregation and | |
| | | | | toxicity induced by | |
| | | | | familial ALS- | |
| | | | | linked mutant | |
| | | | | forms of SOD1, | |
| | | | | Neurobiol. Dis. | |
| | | | | (2013), NCBI | |
| | | | | Accession # | |
| | | | | AGK37119.1 | |
| MUS485 | SOD1 | scFv | 2A10 construct | Ghadge, G.D. et | 6131 |
| | | | | al., Single chain | |
| | | | | variable fragment | |
| | | | | antibodies block | |
| | | | | aggregation and | |
| | | | | toxicity induced by | |
| | | | | familial ALS- | |
| | | | | linked mutant | |
| | | | | forms of SOD1, | |
| | | | | Neurobiol. Dis. | |
| | | | | (2013), NCBI | |
| | | | | Accession # | |
| | | | | AGK37120.1 | |

### Neuropathy Antibodies

In some embodiments, the payload region of the viral particle comprises one or more nucleic acid sequences encoding one or more of the neuropathy payload antibody polypeptides listed in Table 7 (NEURO1-NEURO65; SEQ ID NO:6132-6196).

TABLE 7
Neuropathy Antibodies

| Antibody No. | Target | Description | Antibody Name | Reference Information | SEQ ID NO |
|---|---|---|---|---|---|
| NEURO1 | trk-C (NT-3 | Heavy chain | 2250 | U.S. Pat. No. 7,615,383 | 6132 |
| | trkC ligand) | | | SEQ ID NO: 42 | |
| NEURO2 | trk-C (NT-3 | Heavy chain | 2253 | U.S. Pat. No. 7,615,383 | 6133 |
| | trkC ligand) | | | SEQ ID NO: 43 | |
| NEURO3 | trk-C (NT-3 | Heavy chain | 2256 | U.S. Pat. No. 7,615,383 | 6134 |
| | trkC ligand) | | | SEQ ID NO: 44 | |
| NEURO4 | trk-C (NT-3 | Heavy chain | 6.1.2 | U.S. Pat. No. 7,615,383 | 6135 |
| | trkC ligand) | | | SEQ ID NO: 45 | |
| NEURO5 | trk-C (NT-3 | Heavy chain | 6.4.1 | U.S. Pat. No. 7,615,383 | 6136 |
| | trkC ligand) | | | SEQ ID NO: 46 | |
| NEURO6 | trk-C (NT-3 | Heavy chain | 2345 | U.S. Pat. No. 7,615,383 | 6137 |
| | trkC ligand) | | | SEQ ID NO: 47 | |
| NEURO7 | trk-C (NT-3 | Heavy chain | 2349 | U.S. Pat. No. 7,615,383 | 6138 |
| | trkC ligand) | | | SEQ ID NO: 48 | |
| NEURO8 | RTN4 (NOGO) | Heavy chain IgG4, | Atinumab | U.S. Pat. No. 8,163,285 | 6139 |
| | | immunomodulator | | SEQ ID NO: 24 | |
| NEURO9 | LPG | Heavy chain | #7 | U.S. Pat. No. 8,591,902 | 6140 |
| | (lysophosphatidyl- | variable region | | SEQ ID NO: 18 | |
| | glucoside) | | | | |
| NEURO10 | LPG | Heavy chain | #15 | U.S. Pat. No. 8,591,902 | 6141 |
| | (lysophosphatidyl- | variable region | | SEQ ID NO: 8 | |
| | glucoside) | | | | |
| NEURO11 | MAG | Heavy chain | | U.S. Pat. No. 8,071,731 | 6142 |
| | | variable region | | SEQ ID NO: 13 | |
| NEURO12 | MAG | Heavy chain | | U.S. Pat. No. 8,071,731 | 6143 |
| | | variable region | | SEQ ID NO: 14 | |
| NEURO13 | MAG | Heavy chain | | U.S. Pat. No. 8,071,731 | 6144 |
| | | variable region | | SEQ ID NO: 15 | |
| NEURO14 | MAI (myelin | Heavy chain | | WO2013158748 | 6145 |
| | associated | variable region | | SEQ ID NO: 1 | |
| | inhibitor) | | | | |
| NEURO15 | MAI (myelin | Heavy chain | | WO2013158748 | 6146 |
| | associated | variable region | | SEQ ID NO: 17 | |
| | inhibitor) | | | | |
| NEURO16 | RAGE protein | Heavy chain | Mab 7F9 | US20130149313 | 6147 |
| | | variable region | | SEQ ID NO: 1 | |
| NEURO17 | RAGE protein | Heavy chain | Mab 4E5 | US20130149313 | 6148 |
| | | variable region | | SEQ ID NO: 17 | |
| NEURO18 | RAGE protein | Heavy chain | Mab 11E6 | US20130149313 | 6149 |
| | | variable region | | SEQ ID NO: 9 | |
| NEURO19 | RGMa | Heavy chain | AE12-1 | US20140023659 | 6150 |
| | | variable region | | SEQ ID NO: 1 | |
| NEURO20 | RGMa | Heavy chain | AE12-20 | US20140023659 | 6151 |
| | | variable region | | SEQ ID NO: 107 | |
| NEURO21 | RGMa | Heavy chain | AE12-21 | US20140023659 | 6152 |
| | | variable region | | SEQ ID NO: 115 | |
| NEURO22 | RGMa | Heavy chain | AE12-23 | US20140023659 | 6153 |
| | | variable region | | SEQ ID NO: 123 | |
| NEURO23 | RGMa | Heavy chain | AE12-24 | US20140023659 | 6154 |
| | | variable region | | SEQ ID NO: 131 | |
| NEURO24 | RGMa | Heavy chain | AE12-3 | US20140023659 | 6155 |
| | | variable region | | SEQ ID NO: 17 | |
| NEURO25 | RGMa | Heavy chain | AE12-4 | US20140023659 | 6156 |
| | | variable region | | SEQ ID NO: 25 | |
| NEURO26 | RGMa | Heavy chain | AE12-5 | US20140023659 | 6157 |
| | | variable region | | SEQ ID NO: 33 | |
| NEURO27 | RGMa | Heavy chain | AE12-6 | US20140023659 | 6158 |
| | | variable region | | SEQ ID NO: 41 | |
| NEURO28 | RGMa | Heavy chain | AE12-7 | US20140023659 | 6159 |
| | | variable region | | SEQ ID NO: 49 | |
| NEURO29 | RGMa | Heavy chain | AE12-8 | US20140023659 | 6160 |
| | | variable region | | SEQ ID NO: 57 | |
| NEURO30 | RGMa | Heavy chain | AE12-2 | US20140023659 | 6161 |
| | | variable region | | SEQ ID NO: 9 | |
| NEURO31 | RGMa | Heavy chain | AE12-13 | US20140023659 | 6162 |
| | | variable region | | SEQ ID NO: 91 | |
| NEURO32 | RGMa | Heavy chain | AE12-15 | US20140023659 | 6163 |
| | | variable region | | SEQ ID NO: 99 | |
| NEURO33 | trk-C (NT-3 | Light chain | 2250 | U.S. Pat. No. 7,615,383 | 6164 |
| | trkC ligand) | | | SEQ ID NO: 49 | |
| NEURO34 | trk-C (NT-3 | Light chain | 2253 | U.S. Pat. No. 7,615,383 | 6165 |
| | trkC ligand) | | | SEQ ID NO: 50 | |
| NEURO35 | trk-C (NT-3 | Light chain | 2256 | U.S. Pat. No. 7,615,383 | 6166 |
| | trkC ligand) | | | SEQ ID NO: 51 | |
| NEURO36 | trk-C (NT-3 | Light chain | 6.1.2 | U.S. Pat. No. 7,615,383 | 6167 |
| | trkC ligand) | | | SEQ ID NO: 52 | |
| NEURO37 | trk-C (NT-3 | Light chain | 6.4.1 | U.S. Pat. No. 7,615,383 | 6168 |
| | trkC ligand) | | | SEQ ID NO: 53 | |
| NEURO38 | trk-C (NT-3 | Light chain | 2345 | U.S. Pat. No. 7,615,383 | 6169 |
| | trkC ligand) | | | SEQ ID NO: 54 | |
| NEURO39 | trk-C (NT-3 | Light chain | 2349 | U.S. Pat. No. 7,615,383 | 6170 |
| | trkC ligand) | | | SEQ ID NO: 55 | |
| NEURO40 | RTN4 | Light chain IgG4, | Atinumab | U.S. Pat. No. 8,163,285 | 6171 |
| | | immunomodulator | | SEQ ID NO: 25 | |
| NEURO41 | LPG | Light chain | #7 | U.S. Pat. No. 8,591,902 | 6172 |
| | (lysophosphatidyl- | variable region | | SEQ ID NO: 17 | |
| | glucoside) | | | | |
| NEURO42 | LPG | Light chain | #15 | U.S. Pat. No. 8,591,902 | 6173 |
| | (lysophosphatidyl- | variable region | | SEQ ID NO: 7 | |
| | glucoside) | | | | |
| NEURO43 | MAG | Light chain | | U.S. Pat. No. 8,071,731 | 6174 |
| | | variable region. | | SEQ ID NO: 16 | |
| NEURO44 | MAG | Light chain | | U.S. Pat. No. 8,071,731 | 6175 |
| | | variable region | | SEQ ID NO: 17 | |
| NEURO45 | MAG | Light chain | | U.S. Pat. No. 8,071,731 | 6176 |
| | | variable region | | SEQ ID NO: 18 | |
| NEURO46 | MAG | Light chain | | U.S. Pat. No. 8,071,731 | 6177 |
| | | variable region | | SEQ ID NO: 19 | |
| NEURO47 | MAI (myelin | Light chain | | WO2013158748 | 6178 |
| | associated | variable region | | SEQ ID NO: 11 | |
| | inhibitor) | | | | |
| NEURO48 | MAI (myelin | Light chain | | WO2013158748 | 6179 |
| | associated | variable region | | SEQ ID NO: 27 | |
| | inhibitor) | | | | |
| NEURO49 | RAGE protein | Light chain | Mab 11E6 | US20130149313 | 6180 |
| | | variable region | | SEQ ID NO: 13 | |
| NEURO50 | RAGE protein | Light chain | Mab 4E5 | US20130149313 | 6181 |
| | | variable region | | SEQ ID NO: 21 | |
| NEURO51 | RAGE protein | Light chain | Mab 7F9 | US20130149313 | 6182 |
| | | variable region | | SEQ ID NO: 5 | |
| NEURO52 | RGMa | Light chain | AE12-15 | US20140023659 | 6183 |
| | | variable region | | SEQ ID NO: 103 | |
| NEURO53 | RGMa | Light chain | AE12-20 | US20140023659 | 6184 |
| | | variable region | | SEQ ID NO: 111 | |
| NEURO54 | RGMa | Light chain | AE12-21 | US20140023659 | 6185 |
| | | variable region | | SEQ ID NO: 119 | |
| NEURO55 | RGMa | Light chain | AE12-23 | US20140023659 | 6186 |
| | | variable region | | SEQ ID NO: 127 | |
| NEURO56 | RGMa | Light chain | AE12-2 | US20140023659 | 6187 |
| | | variable region | | SEQ ID NO: 13 | |
| NEURO57 | RGMa | Light chain | AE12-24 | US20140023659 | 6188 |
| | | variable region | | SEQ ID NO: 135 | |
| NEURO58 | RGMa | Light chain | AE12-3 | US20140023659 | 6189 |
| | | variable region | | SEQ ID NO: 21 | |
| NEURO59 | RGMa | Light chain | AE12-4 | US20140023659 | 6190 |
| | | variable region | | SEQ ID NO: 29 | |
| NEURO60 | RGMa | Light chain | AE12-5 | US20140023659 | 6191 |
| | | variable region | | SEQ ID NO: 37 | |
| NEURO61 | RGMa | Light chain | AE12-6 | US20140023659 | 6192 |
| | | variable region | | SEQ ID NO: 45 | |
| NEURO62 | RGMa | Light chain | AE12-1 | US20140023659 | 6193 |
| | | variable region | | SEQ ID NO: 5 | |
| NEURO63 | RGMa | Light chain | AE12-7 | US20140023659 | 6194 |
| | | variable region | | SEQ ID NO: 53 | |
| NEURO64 | RGMa | Light chain | AE12-8 | US20140023659 | 6195 |
| | | variable region | | SEQ ID NO: 61 | |
| NEURO65 | RGMa | Light chain | AE12-13 | US20140023659 | 6196 |
| | | variable region | | SEQ ID NO: 95 | |

### Psychiatric Disorder Antibodies

In some embodiments, the payload region of the viral particle comprises one or more nucleic acid sequences encoding one or more of the psychiatric disorder payload antibody polypeptides listed in Table 8 (PSYCH1-PSYCH160; SECS ID NO: 6197-6356),

TABLE 8
Psychiatric Disorder Antibodies

| Antibody No. | Target | Description | Antibody Name | Reference Information | SEQ ID NO |
|---|---|---|---|---|---|
| PSYCH1 | ACTH | Heavy chain | Ab4 | WO2015127288 | 6197 |
| | | | | SEQ ID NO: 121 | |
| PSYCH2 | ACTH | Heavy chain | Ab1.H | WO2015127288 | 6198 |
| | | | | SEQ ID NO: 441 | |
| PSYCH3 | ACTH | Heavy chain | Ab2.H | WO2015127288 | 6199 |
| | | | | SEQ ID NO: 481 | |
| PSYCH4 | ACTH | Heavy chain | Ab3.H | WO2015127288 | 6200 |
| | | | | SEQ ID NO: 521 | |
| PSYCH5 | ACTH | Heavy chain | Ab4.H | WO2015127288 | 6201 |
| | | | | SEQ ID NO: 561 | |
| PSYCH6 | ACTH | Heavy chain | Ab6.H | WO2015127288 | 6202 |
| | | | | SEQ ID NO: 601 | |
| PSYCH7 | ACTH | Heavy chain | Ab7.H | WO2015127288 | 6203 |
| | | | | SEQ ID NO: 641 | |
| PSYCH8 | ACTH | Heavy chain | Ab7A.H | WO2015127288 | 6204 |
| | | | | SEQ ID NO: 681 | |
| PSYCH9 | ACTH | Heavy chain | Ab10.H | WO2015127288 | 6205 |
| | | | | SEQ ID NO: 721 | |
| PSYCH10 | ACTH | Heavy chain | Ab11.H | WO2015127288 | 6206 |
| | | | | SEQ ID NO: 761 | |
| PSYCH11 | ACTH | Heavy chain | Ab11A.H | WO2015127288 | 6207 |
| | | | | SEQ ID NO: 801 | |
| PSYCH12 | ACTH | Heavy chain | Ab5 | WO2015127288 | 6208 |
| | | | | SEQ ID NO: 161 | |
| PSYCH13 | ACTH | Heavy chain | Ab3 | WO2015127288 | 6209 |
| | | | | SEQ ID NO: 81 | |
| PSYCH14 | ACTH | Heavy chain | Ab12.H | WO2015127288 | 6210 |
| | | | | SEQ ID NO: 841 | |
| PSYCH15 | ACTH | Heavy chain | Ab6 | WO2015127288 | 6211 |
| | | | | SEQ ID NO: 201 | |
| PSYCH16 | ACTH | Heavy chain | Ab7 | WO2015127288 | 6212 |
| | | | | SEQ ID NO: 241 | |
| PSYCH17 | ACTH | Heavy chain | Ab9 | WO2015127288 | 6213 |
| | | | | SEQ ID NO: 281 | |
| PSYCH18 | ACTH | Heavy chain | Ab10 | WO2015127288 | 6214 |
| | | | | SEQ ID NO: 321 | |
| PSYCH19 | ACTH | Heavy chain | Ab11 | WO2015127288 | 6215 |
| | | | | SEQ ID NO: 361 | |
| PSYCH20 | ACTH | Heavy chain | Ab12 | WO2015127288 | 6216 |
| | | | | SEQ ID NO: 401 | |
| PSYCH21 | ACTH | Heavy chain | Ab2 | WO2015127288 | 6217 |
| | | | | SEQ ID NO: 41 | |
| PSYCH22 | ACTH | Heavy chain | Ab1 | WO2015127288 | 6218 |
| | | | | SEQ ID NO: 1 | |
| PSYCH23 | neuregulin | Heavy chain | | US20140363438 | 6219 |
| | (NRG) | | | SEQ ID NO: 72 | |
| PSYCH24 | neuregulin | Heavy chain | | US20140363438 | 6220 |
| | (NRG) | | | SEQ ID NO: 74 | |
| PSYCH25 | Anx-A1 | Heavy chain | VJ-4B6 | US20150004164 | 6221 |
| | | variable region | | SEQ ID NO: 16 | |
| PSYCH26 | Anx-A1 | Heavy chain | VJ-4B6 | US20150004164 | 6222 |
| | | variable region | | SEQ ID NO: 20 | |
| PSYCH27 | RGM A | Heavy chain | 5F9.1-GL | US20150183871 | 6223 |
| | | variable region | | SEQ ID NO: 35 | |
| PSYCH28 | RGM A | Heavy chain | 5F9.2-GL | US20150183871 | 6224 |
| | | variable region | | SEQ ID NO: 36 | |
| PSYCH29 | RGM A | Heavy chain | 5F9.3-GL | US20150183871 | 6225 |
| | | variable region | | SEQ ID NO: 37 | |
| PSYCH30 | RGM A | Heavy chain | 5F9.4-GL | US20150183871 | 6226 |
| | | variable region | | SEQ ID NO: 38 | |
| PSYCH31 | RGM A | Heavy chain | 5F9.5-GL | US20150183871 | 6227 |
| | | variable region | | SEQ ID NO: 39 | |
| PSYCH32 | RGM A | Heavy chain | 5F9.6-GL | US20150183871 | 6228 |
| | | variable region | | SEQ ID NO: 40 | |
| PSYCH33 | RGM A | Heavy chain | 5F9.7-GL | US20150183871 | 6229 |
| | | variable region | | SEQ ID NO: 41 | |
| PSYCH34 | RGM A | Heavy chain | 5F9.8-GL | US20150183871 | 6230 |
| | | variable region | | SEQ ID NO: 42 | |
| PSYCH35 | RGM A | Heavy chain | 5F9.9-G.L | US20150183871 | 6231 |
| | | variable region | | SEQ ID NO: 43 | |
| PSYCH36 | RGM A | Heavy chain | h5F9.1, h5F9.1, h5F9.1, | US20150183871 | 6232 |
| | | variable region | h5F9.1, h5F9.1, h5F9.2, | SEQ ID NO: 47 | |
| | | | h5F9.3 | | |
| PSYCH37 | RGM A | Heavy chain | h5F9.3, h5F9.9, | US20150183871 | 6233 |
| | | variable region | h5F9.25 | SEQ ID NO: 53 | |
| PSYCH38 | RGM A | Heavy chain | h5F9.4, h5F9.10, | US20150183871 | 6234 |
| | | variable region | h5F9.26 | SEQ ID NO: 54 | |
| PSYCH39 | RGMa | Heavy chain | AE12-1 | US20140023659 | 6235 |
| | | variable region | | SEQ ID NO: 1 | |
| PSYCH40 | RGMa | Heavy chain | AE12-20 | US20140023659 | 6236 |
| | | variable region | | SEQ ID NO: 107 | |
| PSYCH41 | RGMa | Heavy chain | AE12-21 | US20140023659 | 6237 |
| | | variable region | | SEQ ID NO: 115 | |
| PSYCH42 | RGMa | Heavy chain | AE12-23 | US20140023659 | 6238 |
| | | variable region | | SEQ ID NO: 123 | |
| PSYCH43 | RGMa | Heavy chain | AE12-24 | US20140023659 | 6239 |
| | | variable region | | SEQ ID NO: 131 | |
| PSYCH44 | RGMa | Heavy chain | AE12-3 | US20140023659 | 6240 |
| | | variable region | | SEQ ID NO: 17 | |
| PSYCH45 | RGMa | Heavy chain | AE12-4 | US20140023659 | 6241 |
| | | variable region | | SEQ ID NO: 25 | |
| PSYCH46 | RGMa | Heavy chain | AE12-5 | US20140023659 | 6242 |
| | | variable region | | SEQ ID NO: 33 | |
| PSYCH47 | RGMa | Heavy chain | AE12-6 | US20140023659 | 6243 |
| | | variable region | | SEQ ID NO: 41 | |
| PSYCH48 | RGMa | Heavy chain | AE12-7 | US20140023659 | 6244 |
| | | variable region | | SEQ ID NO: 49 | |
| PSYCH49 | RGMa | Heavy chain | AE12-8 | US20140023659 | 6245 |
| | | variable region | | SEQ ID NO: 57 | |
| PSYCH50 | RGMa | Heavy chain | AE12-2 | US20140023659 | 6246 |
| | | variable region | | SEQ ID NO: 9 | |
| PSYCH51 | RGMa | Heavy chain | AE12-13 | US20140023659 | 6247 |
| | | variable region | | SEQ ID NO: 91 | |
| PSYCH52 | RGMa | Heavy chain | AE12-15 | US20140023659 | 6248 |
| | | variable region | | SEQ ID NO: 99 | |
| PSYCH53 | TMEFE2 | Heavy chain | PQ01 | US20150030602 | 6249 |
| | | variable region | | SEQ ID NO: 10 | |
| PSYCH54 | TNFa | Heavy chain | 2SD4 | US20140296493 | 6250 |
| | | variable region | | SEQ ID NO: 10 | |
| PSYCH55 | TNFa | Heavy chain | D2E7 | US20140296493 | 6251 |
| | | variable region | | SEQ ID NO: 2 | |
| PSYCH56 | ghrelin | Heavy chain | | US20060233788 | 6252 |
| | | variable region | | SEQ ID NO: 12 | |
| PSYCH57 | ghrelin | Heavy chain | | US20060233788 | 6253 |
| | | variable region | | SEQ ID NO: 13 | |
| PSYCH58 | ghrelin | Heavy chain | | US20060233788 | 6254 |
| | | variable region | | SEQ ID NO: 32 | |
| PSYCH59 | ghrelin | Heavy chain | | US20060233788 | 6255 |
| | | variable region | | SEQ ID NO: 33 | |
| PSYCH60 | neuregulin | Heavy chain | | US20140363438 | 6256 |
| | (NRG) | variable region | | SEQ ID NO: 21 | |
| PSYCH61 | neuregulin | Heavy chain | | US20140363438 | 6257 |
| | (NRG) | variable region | | SEQ ID NO: 52 | |
| PSYCH62 | neuregulin | Heavy chain | | US20140363438 | 6258 |
| | (NRG) | variable region | | SEQ ID NO: 54 | |
| PSYCH63 | neuregulin | Heavy chain | | US20140363438 | 6259 |
| | (NRG) | variable region | | SEQ ID NO: 56 | |
| PSYCH64 | neuregulin | Heavy chain | | US20140363438 | 6260 |
| | (NRG) | variable region | | SEQ ID NO: 58 | |
| PSYCH65 | neuregulin | Heavy chain | | US20140363438 | 6261 |
| | (NRG) | variable region | | SEQ ID NO: 60 | |
| PSYCH66 | neuregulin | Heavy chain | | US20140363438 | 6262 |
| | (NRG) | variable region | | SEQ ID NO: 62 | |
| PSYCH67 | neuregulin | Heavy chain | | US20140363438 | 6263 |
| | (NRG) | variable region | | SEQ ID NO: 63 | |
| PSYCH68 | neuregulin | Heavy chain | | US20140363438 | 6264 |
| | (NRG) | variable region | | SEQ ID NO: 64 | |
| PSYCH69 | neuregulin | Heavy chain | | US20140363438 | 6265 |
| | (NRG) | variable region | | SEQ ID NO: 66 | |
| PSYCH70 | neuregulin | Heavy chain | | US20140363438 | 6266 |
| | (NRG) | variable region | | SEQ ID NO: 68 | |
| PSYCH71 | neuregulin | Heavy chain | | US20140363438 | 6267 |
| | (NRG) | variable region | | SEQ ID NO: 70 | |
| PSYCH72 | ACTH | Light chain | Ab3 | WO2015127288 | 6268 |
| | | | | SEQ ID NO: 101 | |
| PSYCH73 | ACTH | Light chain | Ab4 | WO2015127288 | 6269 |
| | | | | SEQ ID NO: 141 | |
| PSYCH74 | ACTH | Light chain | Ab5 | WO2015127288 | 6270 |
| | | | | SEQ ID NO: 181 | |
| PSYCH75 | ACTH | Light chain | Ab1 | WO2015127288 | 6271 |
| | | | | SEQ ID NO: 21 | |
| PSYCH76 | ACTH | Light chain | Ab6 | WO2015127288 | 6272 |
| | | | | SEQ ID NO: 221 | |
| PSYCH77 | ACTH | Light chain | Ab7 | WO2015127288 | 6273 |
| | | | | SEQ ID NO: 261 | |
| PSYCH78 | ACTH | Light chain | Ab9 | WO2015127288 | 6274 |
| | | | | SEQ ID NO: 301 | |
| PSYCH79 | ACTH | Light chain | Ab10 | WO2015127288 | 6275 |
| | | | | SEQ ID NO: 341 | |
| PSYCH80 | ACTH | Light chain | Ab11 | WO2015127288 | 6276 |
| | | | | SEQ ID NO: 381 | |
| PSYCH81 | ACTH | Light chain | Ab12 | WO2015127288 | 6277 |
| | | | | SEQ ID NO: 421 | |
| PSYCH82 | ACTH | Light chain | Ab1.H | WO2015127288 | 6278 |
| | | | | SEQ ID NO: 461 | |
| PSYCH83 | ACTH | Light chain | Ab2.H | WO2015127288 | 6279 |
| | | | | SEQ ID NO: 501 | |
| PSYCH84 | ACTH | Light chain | Ab3.H | WO2015127288 | 6280 |
| | | | | SEQ ID NO: 541 | |
| PSYCH85 | ACTH | Light chain | Ab4.H | WO2015127288 | 6281 |
| | | | | SEQ ID NO: 581 | |
| PSYCH86 | ACTH | Light chain | Ab2 | WO2015127288 | 6282 |
| | | | | SEQ ID NO: 61 | |
| PSYCH87 | ACTH | Light chain | Ab6.H | WO2015127288 | 6283 |
| | | | | SEQ ID NO: 621 | |
| PSYCH88 | ACTH | Light chain | Ab7.H | WO2015127288 | 6284 |
| | | | | SEQ ID NO: 661 | |
| PSYCH89 | ACTH | Light chain | Ab7A.H | WO2015127288 | 6285 |
| | | | | SEQ ID NO: 701 | |
| PSYCH90 | ACTH | Light chain | Ab10.H | WO2015127288 | 6286 |
| | | | | SEQ ID NO: 741 | |
| PSYCH91 | ACTH | Light chain | Ab11.H | WO2015127288 | 6287 |
| | | | | SEQ ID NO: 781 | |
| PSYCH92 | ACTH | Light chain | Ab11A.H | WO2015127288 | 6288 |
| | | | | SEQ ID NO: 821 | |
| PSYCH93 | ACTH | Light chain | Ab12.H | WO2015127288 | 6289 |
| | | | | SEQ ID NO: 861 | |
| PSYCH94 | neuregulin | Light chain | | US20140363438 | 6290 |
| | (NRG) | | | SEQ ID NO: 73 | |
| PSYCH95 | neuregulin | Light chain | | US20140363438 | 6291 |
| | (NRG) | | | SEQ ID NO: 75 | |
| PSYCH96 | Anx-A1 | Light chain | VJ-4B6 | US20150004164 | 6292 |
| | | variable region | | SEQ ID NO: 15 | |
| PSYCH97 | Anx-A1 | Light chain | VJ-4B6 | US20150004164 | 6293 |
| | | variable region | | SEQ ID NO: 19 | |
| PSYCH98 | RGM A | Light chain | 5F9.1-GL, 5F9.1-GL, | US20150183871 | 6294 |
| | | variable region | 5F9.1-GL, 5F9.1-GL, | SEQ ID NO: 44 | |
| | | | 5F9.1-GL, 5F9.1-GL, | | |
| | | | 5F9.1-GL, 5F9.1-GL, | | |
| | | | 5F9.1-GL, 5F9.1-GL, | | |
| | | | h5F9.4, h5F9.11, | | |
| | | | h5F9.12 | | |
| PSYCH99 | RGM A | Light chain | 5F9.2-GL, 5F9.2-GL, | US20150183871 | 6295 |
| | | variable region | 5F9.2-GL, 5F9.2-GL, | SEQ ID NO: 45 | |
| | | | 5F9.2-GL, 5F9.2-GL, | | |
| | | | 5F9.2-GL, 5F9.2-GL, | | |
| | | | 5F9.2-GL, 5F9.2-GL, | | |
| | | | h5F9.5, h5F9.19, | | |
| | | | h5F9.20 | | |
| PSYCH100 | RGM A | Light chain | 5F9.3-GL, 5F9.3-GL, | US20150183871 | 6296 |
| | | variable region | 5F9.3-GL, 5F9.3-GL, | SEQ ID NO: 46 | |
| | | | 5F9.3-GL, 5F9.3-GL, | | |
| | | | 5F9.3-GL, 5F9.3-GL, | | |
| | | | 5F9.3-GL, 5F9.3-GL, | | |
| | | | h5F9.6, h5F9.21, | | |
| | | | h5F9.22 | | |
| PSYCH101 | RGM A | Light chain | h5F9.5, h5F9.6, h5F9.7, | US20150183871 | 6297 |
| | | variable region | h5F9.8, h5F9.9, | SEQ ID NO: 48 | |
| | | | h5F9.10 | | |
| PSYCH102 | RGM A | Light chain | h5F9.11, | US20150183871 | 6298 |
| | | variable region | h5F9.19, h5F9.21 | SEQ ID NO: 49 | |
| PSYCH103 | RGM A | Light chain | h5F9.12, h5F9.20, | US20150183871 | 6299 |
| | | variable region | h5F9.22, h5F9.23, | SEQ ID NO: 50 | |
| | | | h5F9.25, h5F9.25, | | |
| | | | h5F9.26 | | |
| PSYCH104 | RGM A | Light chain | h5F9.1, h5F9.7, | US20150183871 | 6300 |
| | | variable region | h5F9.23 | SEQ ID NO: 51 | |
| PSYCH105 | RGM A | Light chain | h5F9.2, h5F9.8, | US20150183871 | 6301 |
| | | variable region | h5F9.25 | SEQ ID NO: 52 | |
| PSYCH106 | RGMa | Light chain | AE12-15 | US20140023659 | 6302 |
| | | variable region | | SEQ ID NO: 103 | |
| PSYCH107 | RGMa | Light chain | AE12-20 | US20140023659 | 6303 |
| | | variable region | | SEQ ID NO: 111 | |
| PSYCH108 | RGMa | Light chain | AE12-21 | US20140023659 | 6304 |
| | | variable region | | SEQ ID NO: 119 | |
| PSYCH109 | RGMa | Light chain | AE12-23 | US20140023659 | 6305 |
| | | variable region | | SEQ ID NO: 127 | |
| PSYCH110 | RGMa | Light chain | AE12-2 | US20140023659 | 6306 |
| | | variable region | | SEQ ID NO: 13 | |
| PSYCH111 | RGMa | Light chain | AE12-24 | US20140023659 | 6307 |
| | | variable region | | SEQ ID NO: 135 | |
| PSYCH112 | RGMa | Light chain | AE12-3 | US20140023659 | 6308 |
| | | variable region | | SEQ ID NO: 21 | |
| PSYCH113 | RGMa | Light chain | AE12-4 | US20140023659 | 6309 |
| | | variable region | | SEQ ID NO: 29 | |
| PSYCH114 | RGMa | Light chain | AE12-5 | US20140023659 | 6310 |
| | | variable region | | SEQ ID NO: 37 | |
| PSYCH115 | RGMa | Light chain | AE12-6 | US20140023659 | 6311 |
| | | variable region | | SEQ ID NO: 45 | |
| PSYCH116 | RGMa | Light chain | AE12-1 | US20140023659 | 6312 |
| | | variable region | | SEQ ID NO: 5 | |
| PSYCH117 | RGMa | Light chain | AE12-7 | US20140023659 | 6313 |
| | | variable region | | SEQ ID NO: 53 | |
| PSYCH118 | RGMa | Light chain | AE12-8 | US20140023659 | 6314 |
| | | variable region | | SEQ ID NO: 61 | |
| PSYCH119 | RGMa | Light chain | AE12-13 | US20140023659 | 6315 |
| | | variable region | | SEQ ID NO: 95 | |
| PSYCH120 | TMEFE3 | Light chain | PQ01 | US20150030602 | 6316 |
| | | variable region | | SEQ ID NO: 12 | |
| PSYCH121 | TNFa | Light chain | D2E7 | US20140296493 | 6317 |
| | | variable region | | SEQ ID NO: 1 | |
| PSYCH122 | TNFa | Light chain | 2SD4 | US20140296493 | 6318 |
| | | variable region | | SEQ ID NO: 9 | |
| PSYCH123 | ghrelin | Light chain | | US20060233788 | 6319 |
| | | variable region | | SEQ ID NO: 3 | |
| PSYCH124 | ghrelin | Light chain | | US20060233788 | 6320 |
| | | variable region | | SEQ ID NO: 30 | |
| PSYCH125 | ghrelin | Light chain | | US20060233788 | 6321 |
| | | variable region | | SEQ ID NO: 31 | |
| PSYCH126 | ghrelin | Light chain | | US20060233788 | 6322 |
| | | variable region | | SEQ ID NO: 4 | |
| PSYCH127 | neuregulin | Light chain | | US20140363438 | 6323 |
| | (NRG) | variable region | | SEQ ID NO: 22 | |
| PSYCH128 | neuregulin | Light chain | | US20140363438 | 6324 |
| | (NRG) | variable region | | SEQ ID NO: 23 | |
| PSYCH129 | neuregulin | Light chain | | US20140363438 | 6325 |
| | (NRG) | variable region | | SEQ ID NO: 24 | |
| PSYCH130 | neuregulin | Light chain | | US20140363438 | 6326 |
| | (NRG) | variable region | | SEQ ID NO: 25 | |
| PSYCH131 | neuregulin | Light chain | | US20140363438 | 6327 |
| | (NRG) | variable region | | SEQ ID NO: 26 | |
| PSYCH132 | neuregulin | Light chain | | US20140363438 | 6328 |
| | (NRG) | variable region | | SEQ ID NO: 27 | |
| PSYCH133 | neuregulin | Light chain | | US20140363438 | 6329 |
| | (NRG) | variable region | | SEQ ID NO: 53 | |
| PSYCH134 | neuregulin | Light chain | | US20140363438 | 6330 |
| | (NRG) | variable region | | SEQ ID NO: 55 | |
| PSYCH135 | neuregulin | Light chain | | US20140363438 | 6331 |
| | (NRG) | variable region | | SEQ ID NO: 57 | |
| PSYCH136 | neuregulin | Light chain | | US20140363438 | 6332 |
| | (NRG) | variable region | | SEQ ID NO: 59 | |
| PSYCH137 | neuregulin | Light chain | | US20140363438 | 6333 |
| | (NRG) | variable region | | SEQ ID NO: 61 | |
| PSYCH138 | neuregulin | Light chain | | US20140363438 | 6334 |
| | (NRG) | variable region | | SEQ ID NO: 65 | |
| PSYCH139 | neuregulin | Light chain | | US20140363438 | 6335 |
| | (NRG) | variable region | | SEQ ID NO: 67 | |
| PSYCH140 | neuregulin | Light chain | | US20140363438 | 6336 |
| | (NRG) | variable region | | SEQ ID NO: 69 | |
| PSYCH141 | neuregulin | Light chain | | US20140363438 | 6337 |
| | (NRG) | variable region | | SEQ ID NO: 71 | |
| PSYCH142 | neurokinin B | Single chain scFv | N024C01 | U.S. Pat. No. 7,514,079 | 6338 |
| | | | | SEQ ID NO: 22 | |
| PSYCH143 | neurokinin B | Single chain scFv | N025B07 | U.S. Pat. No. 7,514,079 | 6339 |
| | | | | SEQ ID NO: 23 | |
| PSYCH144 | neurokinin B | Single chain scFv | N015E08 | U.S. Pat. No. 7,514,079 | 6340 |
| | | | | SEQ ID NO: 24 | |
| PSYCH145 | neurokinin B | Single chain scFv | N015F10 | U.S. Pat. No. 7,514,079 | 6341 |
| | | | | SEQ ID NO: 25 | |
| PSYCH146 | neurokinin B | Single chain scFv | N024D01 | U.S. Pat. No. 7,514,079 | 6342 |
| | | | | SEQ ID NO: 26 | |
| PSYCH147 | neurokinin B | Single chain scFv | N015D08 | U.S. Pat. No. 7,514,079 | 6343 |
| | | | | SEQ ID NO: 27 | |
| PSYCH148 | neurokinin B | Single chain scFv | N024B07 | U.S. Pat. No. 7,514,079 | 6344 |
| | | | | SEQ ID NO: 28 | |
| PSYCH149 | neurokinin B | Single chain scFv | N024E07 | U.S. Pat. No. 7,514,079 | 6345 |
| | | | | SEQ ID NO: 29 | |
| PSYCH150 | neurokinin B | Single chain scFv | N023F05 | U.S. Pat. No. 7,514,079 | 6346 |
| | | | | SEQ ID NO: 30 | |
| PSYCH151 | neurokinin B | Single chain scFv | N024D08 | U.S. Pat. No. 7,514,079 | 6347 |
| | | | | SEQ ID NO: 31 | |
| PSYCH152 | neurokinin B | Single chain scFv | N023B03 | U.S. Pat. No. 7,514,079 | 6348 |
| | | | | SEQ ID NO: 32 | |
| PSYCH153 | neurokinin B | Single chain scFv | N023E01 | U.S. Pat. No. 7,514,079 | 6349 |
| | | | | SEQ ID NO: 33 | |
| PSYCH154 | neurokinin B | Single chain scFv | N024C05 | U.S. Pat. No. 7,514,079 | 6350 |
| | | | | SEQ ID NO: 34 | |
| PSYCH155 | neurokinin B | Single chain scFv | N025E05 | U.S. Pat. No. 7,514,079 | 6351 |
| | | | | SEQ ID NO: 35 | |
| PSYCH156 | neurokinin B | Single chain scFv | N025C01 | U.S. Pat. No. 7,514,079 | 6352 |
| | | | | SEQ ID NO: 36 | |
| PSYCH157 | neurokinin B | Single drain scFv | N024F09 | U.S. Pat. No. 7,514,079 | 6353 |
| | | | | SEQ ID NO: 37 | |
| PSYCH158 | neurokinin B | Single chain scFv | N024B01 | U.S. Pat. No. 7,514,079 | 6354 |
| | | | | SEQ ID NO: 38 | |
| PSYCH159 | neurokinin B | Single chain scFv | N024F07 | U.S. Pat. No. 7,514,079 | 6355 |
| | | | | SEQ ID NO: 39 | |
| PSYCH160 | neurokinin B | Single chain scFv | N015D10 | U.S. Pat. No. 7,514,079 | 6356 |
| | | | | SEQ ID NO: 40 | |

### Cancer, Inflammation and Immune System Antibodies

In some embodiments, the payload region of the viral particle comprises one or more nucleic acid sequences encoding one or more of the cancer, inflammation and immune system payload antibody polypeptides listed in Table 9 (CII1-CII3310; SEQ ID NO: 6357-19665).

Lengthy table referenced here
US20240124889A1-20240418-T00001
Please refer to the end of the specification for access instructions.

In Table 9, the target number (Target No.) code is described in the following semi-colon delimited list where the target number is followed by the target (e.g., Target No. 1 with target AC133 is shown as Target No. 1-Target AC133). The targets represented by the codes in Table 9 include, but are not limited to, Target No. 1-Target AC133; Target No. 2-Target ACTH; Target No. 3-Target activin receptor-like kinase 1 (ALK-1); Target No. 4-Target ADAMTS4; Target No. 5-Target AFP; Target No. 6-Target Albumin; Target No. 7-Target ALCM; Target No. 8-Target alpha-4 integrin; Target No. 9-Target angiopoietin 2 (ANGPT2; ANG-2); Target No. 10-Target angiopoietin 2 (ANGPT2; ANG-2) (ANGPT2; ANG-2); Target No. 11-Target Annexin IV or a phospholipid; and (b) a complement inhibitor; Target No. 12-Target Anti-CD-3; Target No. 13-Target antiHER2; Target No. 14-Target anti-Her2 and anti-Her3; Target No. 15-Target antiHER3; Target No. 16-Target anti-idiotype (Id); Target No. 17-Target Anx-A1; Target No, 18-Target A0C3 (VAP-1); Target No. 19-Target Alpha-V integrin; Target No. 20-Target AXT; Target No. 21-Target B and T human lymphocytes; Target No. 22-Target b7 subunit of a4b7, aEb7 integrins, humanized IgG1; Target No. 23-Target B7-H1; Target No. 24-Target B7-H3; Target No. 25-Target B7-H4; Target No. 26-Target B7-H5; Target No. 27-Target B7-H6; Target No. 28-Target B7-H7; Target No. 29-Target B7-H8; Target No, 30-Target BMP9; Target No. 31-Target BSG; Target No. 32-Target C3b; Target No. 33-Target C3b, Properdin (factor P), Factors Ba and Bb, C5, C6, C7, C8, C9; Target No, 34-Target C5; Target No. 35-Target C5a; Target No. 36-Target Cyd polypeptide; Target No, 37-Target CA 125 (MUC16); Target No, 38-Target CA-125 (imitation); Target No. 39-Target C-antigen; Target No. 40-Target Carbohydrate Antigen 242 (CA242); Target No. 41-Target carbonic anhydrase 9(CA-IX); Target No. 42-Target CC chemokines; Target No. 43-Target CCL11 (eotaxin-1); Target No. 44-Target CCL2, MCP-1, MCAF; Target No. 45-Target CCR2; Target No. 46-Target CCR4; Target No. 47-Target CD100; Target No. 48-Target CD11; Target No. 49-Target CD11a; Target No. 50-Target CD123; Target No. 51-Target CD147 (basigin); Target No. 52-Target CD154 (CD40LG); Target No. 53-Target CD19; Target No. 54-Target CD19; Target No. 55-Target CD2; Target No. 56-Target CD20; Target No. 57-Target CD20/CD40; Target No. 58-Target CD20/EGFR; Target No. 59-Target CD200; Target No. 60-Target CD22; Target No. 61-Target CD221; Target No. 62-Target CD248 (TEM-1); Target No. 63-Target CD27; Target No. 64-Target CD274 (PD-L1); Target No. 65-Target CD28; Target No. 66-Target CD3; Target No. 67-Target CD3; Target No. 68-Target CD3 epsilon; Target No. 69-Target CD3 epsilon, anti-IL1-Ri; Target No. 70-Target CD3, CD19; Target No. 71-Target CD3, EpCAM; Target No. 72-Target CD3, MSCP; Target No. 73-Target CD3/CD19 or CD3/CD20; Target No. 74-Target CD3; CD19; Target No. 75-Target CD30; Target No. 76-Target CD31; Target No. 77-Target CD32; Target No. 78-Target CD324/E-cadherin; Target No. 79-Target CD32b; Target No. 80-Target CD33; Target No. 81-Target CD34; Target No. 82-Target CD35; Target No. 83-Target CD37; Target No. 84-Target CD37 and CD20; Target No. 85-Target CD38; Target No. 86-Target CD38, human IgG1; Target No. 87-Target CD38, human IgG2; Target No. 88-Target CD3E; Target No. 89-Target CD3E, EPCAM; Target No. 90-Target CD3E, EPCAM (IL-beta); Target No. 91-Target CD4; Target No. 92-Target CD40; Target No. 93-Target CD40LG; Target No, 94-Target CD44 v6; Target No. 95-Target CD-49d, CD11a; Target No. 96-Target CD51; Target No. 97-Target CD52; Target No. 98-Target CD55/CD59 and CD20; Target No. 99-Target CD6; Target No. 100-Target CD64; Target No. 101 Target CD70; Target No. 102-Target CD74; Target No. 103-Target CD79B; Target No. 104-Target CD89; Target No. 105-Target CEA; Target No. 106-Target CEACAM5; Target No. 107-Target Cell surface targets; Target No. 108-Target CH region of an immunoglobulin; Target No. 109-Target c-MET; Target No, 110-Target c-MET/EGFR; Target No, 111-Target c-MET/EGER; c-MET; Target No. 112-Target c-MET/EGER; EGFR; HGF; Target No. 113-Target c-MET/FGFR; Target No. 114-Target c-MET/EGER; Target No. 115-Target c-MET/EGER; ErbB2; Target No. 116-Target c-MET; EGFR; VEGF; c-MET/EGFR; Target No. 117-Target CSAp; Target No. 118-Target CSF1R; Target No. 119-Target CSF2; Target No. 120-Target CSF2RA; Target No, 121-Target CSPG4; Target No, 122-Target CTGF; Target No. 123-Target CTLA4; Target No. 124-Target CTLA4, human IgG2; Target No. 125-Target CTLA4, human IgG3; Target No. 126-Target C-X-C chemokine receptor type 4; Target No. 127-Target CXCL10; Target No. 128-Target CXCL13; Target No. 129-Target CXCR4; Target No. 130-Target difucosyl Lewis blood group antigens Y-6 and B-7-2; Target No. 131-Target DKK1; Target No. 132-Target DLL3; Target No. 133-Target DLL4; Target No. 134-Target DNA/histone complex; Target No. 135-Target DPP4, CD26; Target No. 136-Target DR5; Target No. 137Target ETNA 1; Target No, 138-Target EGF; Target No, 139-Target EGFL7; Target No. 140-Target EGFR; Target No. 141-Target EGFR (EGERvIII); Target No. 142-Target EGFR (HERD; Target No. 143-Target EGFR and IGF1R; Target No. 144-Target EGFR family; Target No. 145-Target EGFR, ERBB1, HER1; Target No. 146-Target EGFR, ERBB1, HER2; Target No. 147-Target EGFR, HER2, or HER3; Target No. 148-Target EGFR/cMet; Target No. 149-Target EGFR/HER3; Target No. 150-Target EGFR/VEGFR/HER; Target No. 151-Target EGFR; c-Met; Target No. 152-Target EGFR; VEGF; Target No. 153-Target EGFRvIII; Target No. 154-Target EGP-1 (TROP2); Target No. 155-Target EMP2; Target No. 156-Target endoglin; Target No. 157-Target EPCAM; Target No. 158-Target EpCAM, CD3; Target No. 159-Target EphA2 receptor; Target No. 160-Target EPHA3; Target No. 161-Target EphA3; EGFR BER2; PD-L1; HGF; Target No. 162-Target episialin; Target No. 163-Target ERB2; Target No. 164-Target ERBB; Target No. 165-Target ERBB1; Target No. 166-Target ERBB2; Target No. 167-Target ERBB3; Target No. 168-Target ErbB3/IGF1R; Target No. 169-Target ErbB4; Target No. 170-Target ErbB5; Target No. 171-Target ErbB6; Target No. 172-Target ErbB7; Target No. 173-Target ErbB8; Target No. 174-Target euGc, NGNA; Target No. 175-Target F3; Target No. 176-Target FAP; Target No. 177-Target FAN; Target No. 178-Target FasR; Target No. 179-Target FcRn; Target No. 180-Target FcγRIIB (FcγR); Target No, 181-Target FcγRIIB; Target No. 182-Target FcγRIIIA; Target No. 183-Target FGF-8; Target No. 184-Target FGFR2; Target No. 185-Target fibronectin ED-A; Target No. 186-Target fibronectin IIIC isoforms Target No. 187-Target fibronectin extra domain-B; Target No. 188-Target FL Ti; Target No. 189-Target FLT3; Target No. 190-Target folate receptor alpha; Target No. 191-Target FOLR1; Target No. 192-Target Frizzled receptor; Target No. 193-Target ganglioside; Target No. 194-Target GD2; Target No. 195-Target GD2/DOTA; Target No. 196-Target GD2/huOKT3; Target No. 197-Target GD3; Target No. 198-Target GD3 ganglioside; Target No. 199-Target GFRα3; Target No. 200-Target glycan antigen; Target No. 201-Target glypican 3; Target No. 202-Target GM2; Target No. 203-Target GPNMB; Target No. 204-Target Growth factor 7; Target No. 205-Target GUCY2C, anti-GCC; Target No. 206-Target HB-EGF; Target No, 207-Target HB-EGF/EGFR; Target No. 208-Target hen egg lysozyme; Target No. 209-Target HER/EGFR; Target No. 210-Target HER1, HER3, CD80, CD86, PD-1, CTLA4, B7-H4, RON, CD200, CD4, BAF R, EGFR, IGFR VEGFR, a member of the TNF family of receptors, a Tie receptor, MET, IGF1, IGF2, TNF, a TNT ligand, IL-6, TWEAK, Fn14, CD20, CD23, CRIPTO, HGF, alpha4beta1 integrin, alpha5beta1 integrin, alpha6beta4 integrin, and alphaVbeta6 integrin; Target No. 211-Target HER2; Target No. 212-Target HER2/CD3; Target No. 213-Target HER2/Dig; Target No. 214-Target HER2/neu; Target No. 215-Target HER3; Target No. 216-Target HER3, human IgG1; Target No. 217-Target HGF; Target No. 218-Target hIL-12; Target No. 219-Target hIL13; Target No. 220-Target HIV gp120; Target No. 221-Target HLA-DR; Target No. 222-Target hNav1.7; Target No. 223-Target hPG; Target No. 224-Target human TNF; Target No. 225-Target huTNFR; Target No. 226-Target huTNFR1; Target No. 227-Target ICAM-1; Target No. 228-Target IFNAR1; Target No. 229-Target IFN-α; Target No. 230-Target IGF; Target No. 231-Target IGF; IGT1R; Target No. 232-Target IGF1; Target No, 233-Target IGF IR; Target No. 234-Target IGF1R1Dig; Target No. 235-Target IGF-1R/ErbB3; Target No. 236-Target IGF1R; EGFR; Target No. 237-Target IgG4 (CD40); Target No. 238-Target IGHE; Target No. 239-Target IL1; Target No. 240-Target IL10; Target No. 241 Target IL11; Target No. 242-Target IL12; Target No. 243-Target IL12B, IL12 p40, NKSF2, CMLF p40; Target No. 244-Target IL12B, IL12 p40, NKSF2, CMLF p41; Target No. 245-Target 11_12 p40; Target No. 246-Target IL13; Target No. 247-Target IL13, Human IgG4; Target No. 248-Target IL13, Human IgG5; Target No, 249-Target IL17; Target No. 250-Target IL17A; Target No. 251-Target 11,17A and IL17F; Target No. 252-Target IL17RA; Target No. 253-Target IL18; Target No. 254-Target IL18BP; Target No. 255-Target ILIA; Target No. 256-Target LIB; Target No. 257-Target IL20; Target No. 258-Target IL20, NGF; Target No. 259-Target 1122; Target No. 260-Target IL23 A; Target No. 261-Target IL23p19 subunit, humanized IgG1; Target No. 262-Target IL23p19 subunit, humanized IgG2; Target No. 263-Target IL2RA; Target No. 264-Target 11-31RA; Target No, 265-Target IL4; Target No. 266-Target IL4R; Target No. 267-Target IL5; Target No. 268-Target IL5RA; Target No. 269-Target IL6; Target No. 270-Target IL6R; Target No. 271-Target IL6R, humanized IgG2; Target No, 272-Target IL7; Target No. 273-Target IL7R; Target No. 274-Target IL8; Target No. 275-Target IL9; Target No. 276-Target ILGF2; Target No, 277-Target Integrin 2; Target No. 278-Target integrin a4137; Target No, 279-Target integrin (108; Target No, 280-Target IP-10; Target No, 281-Target IS12B; Target No. 282-Target ITGA2; Target No. 283-Target ITGA4_ITGB7; Target No. 284-Target ITGAL; Target No. 285-Target ITGAV_ITGB3; Target No, 286-Target ITGAV_ITGB3; Target No. 287-Target IDR; Target No. 288-Target KIR2; Target No. 289-Target KIR2D; Target No. 290-Target KLRC1; Target No. 291-Target LAG-3; Target No. 292-Target LecLe.sup.x, Le.sup.aLe.sup.x, Di-Le.sup.a, Le.sup.x containing glycans and Le.sup.a containing glycans; Target No. 293-Target Lewis b (LeB); Target No, 294-Target Lewis Y (LeY); Target No. 295-Target LIGHT/HER2/CD23; Target No. 296-Target LIGHT/HER2/CD24; Target No. 297-Target LIGHT/HER2/CD25; Target No. 298-Target LIGHT/HER2/CD26; Target No. 299-Target LIGHT/HER2/CD27; Target No. 300-Target LIGHT/HER2/CD28; Target No. 301-Target LIGHT/HER2/CD29; Target No. 302-Target LIGHT/HER2/CD30; Target No. 303-Target LIGHT/HER2/CD31; Target No. 304-Target LIGHT/HER2/CD32; Target No. 305-Target LINGO-1; Target No. 306-Target LOXL2; Target No. 307-Target LTA; Target No. 308-Target MAGE-A3; Target No. 309-Target MAI (myelin associated inhibitor); Target No. 310-Target many targets; Target No. 311-Target MCP-1; Target No. 312-Target MCP-2; Target No. 313-Target MCP-3; Target No. 314-Target MCP-4; Target No, 315-Target MCP-5; Target No. 316-Target MCP-6; Target No. 317-Target MC SP; Target No. 318-Target MEK; Target No, 319-Target mesothelin; Target No. 320-Target MET; Target No. 321-Target MET Receptor; Target No. 322-Target MHC; Target No. 323-Target MHC class 11; Target No. 324-Target MIF; Target No. 325-Target MMP3; Target No. 326-Target molecules on brain microvascular endothelial cells; Target No. 327-Target monosialo-GM2; Target No. 328-Target MS4A1; Target No. 329-Target MSLN; Target No. 330-Target MST1R; Target No. 331-Target MT4-MMR/EGFR; Target No. 332-Target MTX and EGFR; Target No. 333-Target MTX and hCD-20; Target No, 334-Target MIX and hCD-3; Target No. 335-Target MTX and mCD-3; Target No. 336-Target MUC1; Target No. 337-Target MUC1/MUC5ac; Target No. 338-Target MUC5AC; Target No. 339-Target mucin CanAg; Target No. 340-Target N terminus end of properdin; Target No. 341-Target NCAM1; Target No. 342-Target NeuGc, NGNA; Target No. 343-Target neuregulin (NRG); Target No. 344-Target neurokinin B; Target No. 345-Target neurotensin; Target No. 346-Target NGF; Target No. 347-Target NGF; c-MET; Target No. 348-Target N-glycolyl-GM3; Target No. 349-Target NMDA; Target No. 350-Target NOGO; Target No. 351-Target Nogo receptor-i; Target No. 352-Target Notch receptor; Target No. 353-Target NOTCH1; Target No. 354-Target NRP1; Target No. 355-Target 0-acetylated-GD2; Target No. 356-Target 01′GL; Target No. 357-Target OX-40; Target No. 358-Target oxLDL; Target No. 359-Target PAM4 antigens; Target No. 360-Target PD-1; Target No. 361-Target PD1, human IgG4; Target No. 362-Target PDGFRA; Target No, 363-Target PDGFR-beta; Target No. 364-Target PDGFRβ/VEGFA; Target No. 365-Target PD-L1; Target No. 366-Target PD-Li, human IgG1; Target No. 367-Target PD-L2; Target No. 368-Target periostin; Target No. 369-Target PERP; Target No. 370-Target PhosphatidyL-serine, chimeric IgG1; Target No. 371-Target PhosphatidyL-serine, Chimeric IgG2; Target No. 372-Target polyubiquitin; Target No. 373-Target PSMA; Target No. 374-Target PVRL4; Target No. 375-Target PVRL5; Target No. 376-Target RANKL; Target No, 377-Target RANKL/PTH; Target No. 378-Target RFB4; Target No. 379-Target RON; Target No. 380-Target RTN4 (NOGO); Target No. 381-Target S1P4; Target No. 382-Target SDC1; Target No. 383-Target selectin; Target No. 384-Target Serum albumin (mouse); Target No. 385-Target Serum albumin or neonatal Fc receptor; Target No. 386-Target sialic acid (Neu5Gc or Neu5Ac); Target No. 387-Target sialyl Tn (sTn); Target No. 388-Target Sialyl-Lewis A (sLeA); Target No. 389-Target siaiyltetraosyl carbohydrate (Colo205); Target No. 390-Target SI Pa; Target No. 391-Target SLAMF7; Target No. 392-Target SLC34A2; Target No. 393-Target SOST; Target No. 394-Target STEAP1; Target No. 395-Target sTn; Target No. 396-Target TAC; Target No. 397-Target TAG-72; Target No. 398-Target Tenascin (INC-A1 or TNC-A4); Target No. 399-Target Tenascin (INC-A2); Target No. 400-Target tenascin C; Target No. 401-Target tenascin W; Target No. 402-Target tenascin; Target No. 403-Target Ten-M2; Target No. 404-Target TGF beta 1; Target No. 405-Target TGFbeta; Target No. 406-Target TGF-α; Target No, 407-Target TIGIT; Target No. 408-Target TIM-3; Target No. 409-Target TLR3; Target No. 410-Target Tn antigen; Target No. 411-Target Tn-(ML1C21); Target No. 412-Target TNF; Target No. 413-Target TNFalpha; Target No. 414-Target TNFRSF10B Target No. 415-Target TNFRSF12A; Target No. 416-Target TINFRSF8; Target No. 417-Target TNFRSF9; Target No. 418-Target TNT SF11; Target No. 419-Target TNFSF13B; Target No. 420-Target TPBG; Target No. 421-Target TRAIL-R2; Target No. 422-Target TrkA; Target No. 423-Target TSLP; Target No. 424-Target tumor associated carbohydrate antigen (TACH); Target No. 425-Target tumor specific glycosylation of MUC1, Target No. 426-Target tumor-associated calcium signal transducer 2; Target No. 427-Target TYRP1(glycoprotein 75); Target No. 428-Target VEGF; Target No. 429-Target VEGF, c-Met, CD20; CD38, IL-8, CD25, CD74, FcalphaR1, FcepsilonR1, acetyl choline receptor, fas, fast, TRAIL, hepatitis virus, hepatitis C virus, envelope E2 of hepatitis C virus, tissue factor, a complex of tissue factor and Factor VII, EGFr, CD4, and CD28; Target No. 430-Target VEGFA; Target No. 431-Target VEGFA, ANGT2; Target No, 432-Target VEGFR2; Target No. 433-Target vimentin; Target No. 434-Target VRGF; Target No. 435-Target VSTM5; Target No. 436-Target VWF; Target No. 437-Target a6134 integrin; Target No. 438-Target a-folate receptor, αvβ6integrin, BCMA, B7-E13, B7-H6, CALX, CD19, CD20, CD22, CD30, CD33, CD37, CD44, CD44v6, CD44v7/8, CD70; CD123, CD138, CD171, CEA, DLL4, EGP-2, EGP-40, CSPG4, EGFR, EGER family including ErbB2 (HER2), EGFRvIII, EPCAM, EphA2, EpCAM, RAP, FBP, fetal acetylcholine receptor, Fzd7, GD2, GD3, Glypican-3 (GPC3), h5T4, IL-11Ra, IL13R-α2, KDR, κ light chain, 2 light chain, LeY, L1CAM, MAGE-A1, mesothelin, MHC presented peptides, MUC1, MUC16, NKG2D ligands, Notch1, Notch2/3, NY-ESO-1, PRAME, PSCA, PSMA, Survivin; TAG-72, TEMs, TERT, VEGFR2, and ROR1; and Target No. 439-Target αβv6 integrin.

In Table 9, the description number (Description No.) code is described in the following semi-colon delimited list where the description number is followed by the description (e.g., Description No. 1 with description aglycosylated antibody is shown as Description No. 1-Description aglycosylated antibody). The targets represented by the codes in Table 9 include, but are not limited to, Description No. 1-Descriptionaglycosylated antibody; Description No. 2-DescriptionAmplified variable region; Description No, 3-DescriptionAntibody; Description No. 4-DescriptionAntibody for Pulmonary Fibrosis; Description No. 5-DescriptionBinding peptide; Description No. 6-DescriptionBispecific; Description No. 7-Descriptionbispecific antibody; Description No. 8-DescdptionBR96 say; Description No. 9-DescriptionChain A, Human Igg1 Fc Fragment; Description No. 10-DescriptionChain B, Human Igg1 Fc Fragment; Description No. 11-DescriptionChimeric antigen receptor with cd19 Binding domain; Description No. 12-DescriptionConsensus sequence; Description No. 13-DescriptionConstant region; Description No. 14-DescriptionConstant region IgG1; Description No. 15-DescriptionConstant region IgG2; Description No. 16-DescriptionConstant region IgG3; Description No. 17-DescriptionConstruct; Description No. 18-DescriptionDiabody; Description No. 19-DescriptionDomain antibody; Description No. 20-DescriptiondsFv; Description No. 21-DescdptionDVD heavy chain; Description No. 22-DescriptionDVD light chain; Description No. 23-DescriptionEGFR-specific variable region and CH2 region; Description No. 24-DescriptionFab Heavy chain; Description No. 25-Description Fab heavy chain-Fc; Description No. 26-Description Fc; Description No. 27-Description Fc domain; Description No. 28-Description Fc polypeptide; Description No. 29-Description fc region Igg1; Description No. 30-Description fibronectin type III (FN3) domain; Description No. 31-Description first Fe domain, isoleucine zipper, IgG2 hinge, and second Fc domain; Description No. 32-Description fragment crystallizable region; Description No. 33-Description full sequence; Description No. 34-Description fusion construct; Description No. 35-Description fusion protein; Description No. 36-Description Fusion protein, bispecific; Description No. 37-Description Fusion protein, tumor suppressor protein epha7ecd; Description No, 38-Description Germline Heavy Chain—variable region; Description No. 39-Description Heavy chain variable region; Description No. 40-Description Heavy chain; Description No. 41-Description Heavy chain-constant region; Description No. 42-Description Heavy Chain-variable region; Description No. 43-Description Heavy Chain (Genetic Recombination), Antibody for paroxysmal nocturnal hemoglobinuria; Description No. 44-Description Heavy chain 1; Description No. 45-Description Heavy Chain 1, Antibody for immunosuppressant; Description No. 46-Description Heavy chain 2; Description No, 47-Description Heavy chain A; Description No. 48-Description Heavy chain amino acid sequence humanized; Description No. 49-Description Heavy chain antigen binding region; Description No. 50-Description Heavy chain B; Description No. 51-Description Heavy chain amino acid antibodies; Description No. 52-Description Heavy chain CDR; Description No. 53-Description Heavy Chain CDR 1, immunosuppressant; Description No. 54-Description Heavy Chain CDR 2, immunosuppressant; Description No. 55-Description Heavy Chain CDR 3, immunosuppressant; Description No. 56-Description Heavy chain CDR grafted anti-IL-5; Description No. 57-Description Heavy Chain CDR1; Description No. 58-Description Heavy Chain CDR1, Antibody for paroxysmal nocturnal hemoglobinuria; Description No, 59-Description Heavy chain CDR1, Antibody for rheumatoid arthritis; Description No. 60-Description Heavy Chain CDR 1, immunosuppressant; Description No. 61-Description Heavy Chain CDR2; Description No. 62-Description Heavy Chain CDR2, Antibody for paroxysmal nocturnal hemoglobinuria; Description No, 63-Description Heavy chain CDR2, Antibody for rheumatoid arthritis; Description No. 64-Description Heavy Chain CDR2, immunosuppressant; Description No, 65-Description Heavy Chain CDR3; Description No. 66-Description Heavy Chain CDR3, Antibody for paroxysmal nocturnal hemoglobinuria; Description No. 67-Description Heavy chain CDR3, Antibody for rheumatoid arthritis; Description No. 68-Description Heavy Chain CDR3, immunosuppressant; Description No. 69-Description Heavy chain chimeric; Description No. 70-Description Heavy chain Consensus sequence; Description No. 71-Description Heavy chain constant; Description No. 72-Description heavy chain constant domain; Description No. 73-Description Heavy chain constant gamma-1; Description No. 74-Description Heavy chain constant Ig gamma 1; Description No. 75-Description Heavy chain constant of polypeptide; Description No. 76-Description Heavy chain-constant region Hu1D10-IgG2M3; Description No, 77-Description Heavy chain constant region, human IgG4; Description No. 78-Description Heavy chain constant region, wildtype; Description No. 79-Description Heavy chain constant, CH1; Description No. 80-Description Heavy chain constant, CH2; Description No. 81-Description Heavy chain constant, CH3; Description No. 82-Description Heavy chain constant, human IgG; Description No. 83-Description Heavy chain constant, human IgG4; Description No, 84-Description Heavy chain constant, human IgG4 hingeless; Description No. 85-Description Heavy chain Fab; Description No. 86-Description Heavy chain Fab fragment, Chimeric (anti-alpha2-V_{H}-IGHG1-CH1); Description No. 87-Description Heavy chain gamma consensus sequence; Description No. 88-Description Heavy chain gamma sequence; Description No. 89-Description Heavy chain humanized construct H1; Description No. 90-Description Heavy chain humanized construct H14; Description No. 91-Description Heavy chain humanized construct H15; Description No. 92-Description Heavy chain humanized construct H16; Description No. 93-Description Heavy chain humanized construct H17; Description No. 94-Description Heavy chain humanized construct F118; Description No. 95-Description Heavy chain humanized construct H19; Description No. 96-Description Heavy chain humanized construct H20; Description No. 97-Description Heavy chain humanized construct 1-121; Description No. 98-Description Heavy chain humanized construct H22; Description No. 99-Description Heavy chain humanized construct 1-123; Description No. 100-Description Heavy chain humanized construct H24; Description No. 101-Description Heavy chain humanized construct H25; Description No. 102-Description Heavy chain humanized construct H5; Description No. 103-Description Heavy chain humanized construct H6; Description No, 104-Description Heavy chain humanized construct H700; Description No. 105-Description Heavy chain IgG4, immunomodulator; Description No. 106-Description Heavy chain immunoglobulin variable region; Description No, 107-Description Heavy chain immunoglobulin; Description No. 108 Description Heavy chain leader and variable region of the murine anti-IGF-I receptor antibody; Description No. 109-Description Heavy chain mature; Description No. 110-Description Heavy chain mature fragment; Description No. 111-Description Heavy chain mature immunoglobulin; Description No. 112-Description Heavy chain mature variable region; Description No. 113-Description Heavy chain mature, Antibody for rheumatic diseases; Description No. 114-Description Heavy chain of huAbF46-H4-A1, human IgG2 hinge and constant region of human IgG1; Description No. 115-Description Heavy chain of huAbF464H4-A1, human IgG2 hinge and constant region of human IgG2; Description No. 116-Description Heavy chain of huAbF46-H4-A1, U6-HC7 hinge and constant region of human IgG1; Description No. 117-Description Heavy chain polypeptide; Description No. 118-Description Heavy chain protein; Description No. 119-Description Heavy chain sequence; Description No. 120-Description Heavy chain used in humanization; Description No. 121-Description Heavy chain variable and constant chain; Description No. 122-Description Heavy chain variable domain; Description No. 123-Description heavy chain variable domain H1 AC10; Description No. 124-Description heavy chain variable domain 112 AC11; Description No. 125-Description heavy chain variable domain H3 AC12; Description No. 126-Description heavy chain variable domain L1 AC11; Description No. 127-Description heavy chain variable domain L2 AC12; Description No. 128-Description heavy chain variable domain L3 AC13; Description No. 129-Description Heavy chain variable domain of anti-alpha2-integrin; Description No. 130-Description Heavy chain variable domain of anti-alpha2-integrin mAb; Description No, 131-Description Heavy Chain Variable Domain, Antibody for rheumatoid arthritis, psoriatic arthritis, ankylosing spondylitis, Crohn's disease, ulcerative colitis, moderate to severe chronic psoriasis and juvenile idiopathic arthritis, D2E7; Description No. 132-Description Heavy Chain Variable domain, immunosuppressant for lupus; Description No. 133-Description Heavy chain variable domain, murine; Description No. 134-Description Heavy chain variable of scFv, immunosuppressant for lupus; Description No. 135-Description heavy chain variable region (excludes the heavy chain variable region of the ErbB3 binding site of 160; Description No. 136-Description heavy chain variable region (V_{H}); Description No. 137-Description Heavy chain variable region (with signal sequence removed); Description No. 138-Description Heavy chain variable region 1; Description No. 139-Description Heavy chain variable region 2; Description No. 140-Description heavy chain variable region and heavy chain; Description No. 141-Description Heavy chain variable region and IgG-1 constant region; Description No. 142-Description Heavy chain variable region chain, Antibody for rheumatoid arthritis; Description No. 143-Description Heavy chain variable region consensus framework; Description No, 144-Description Heavy chain variable region domain (as translated) listed in USS 736137; Description No. 145-Description Heavy chain variable region domain chain 1, Anti-IgE antibody; Description No. 146-Description Heavy chain variable region domain, Antibody for Fibrotic diseases, scarring, diffuse scleroderma; Description No. 147-Description heavy chain variable region dual variable domain; Description No. 148-Description Heavy chain variable region humanized construct H1; Description No. 149-Description Heavy chain variable region humanized construct H14; Description No. 150-Description Heavy chain variable region humanized construct H15; Description No. 151-Description Heavy chain variable region humanized construct H16; Description No. 152-Description Heavy chain variable regi on humanized construct H17; Description No. 153-Description Heavy chain variable region humanized construct H18; Description No. 154-Description Heavy chain variable region humanized construct H19; Description No. 155-Description Heavy chain variable region humanized construct H20; Description No. 156-Description Heavy chain variable region humanized construct H21; Description No. 157-Description Heavy chain variable region humanized construct H22; Description No. 158-Description Heavy chain variable region humanized construct H23; Description No. 159-Description Heavy chain variable region humanized construct H24; Description No. 160-Description Heavy chain variable region humanized construct H25; Description No. 161-Description Heavy chain variable region humanized construct H5; Description No. 162-Description Heavy chain variable region humanized construct H6; Description No, 163-Description Heavy chain variable region humanized construct H700; Description No. 164-Description Heavy chain variable region variant; Description No. 165-Description Heavy chain variable region with CDRs and human CR1-hinge-aglycosylCH2CH3; Description No. 166-Description Heavy chain variable region with predicted signal; Description No. 167-Description Heavy chain variable region without predicted signal; Description No. 168-Description Heavy chain variable region without signal; Description No. 169-Description Heavy chain variable region without signal sequence; Description No. 170-Description Heavy chain variable region, Amino acid sequence encoded by the 4-61 gene; Description No. 171-Description Heavy chain variable region, Antibody for acute coronary syndrome, atherosclerosis; Description No. 172-Description Heavy chain variable region, Antibody for allograft rejection; Description No. 173-Description Heavy Chain Variable Region, Antibody for chronic plaque psoriasis; Description No, 174-Description Heavy chain variable region, Antibody for Neuromyelitis optica and NMO Spectrum Disorder; Description No. 175-Description Heavy chain variable region, Antibody for osteoporosis; Description No. 176-Description Heavy chain variable region, Antibody for psoriasis (blocks T-cell migration); Description No. 177-Description Heavy chain variable region, Antibody for Pulmonary Fibrosis; Description No. 178-Description Heavy chain variable region, Antibody for rheumatoid arthritis; Description No. 179-Description Heavy chain variable region, camelid derived; Description No. 180-Description Heavy chain variable region, chimeric; Description No. 181-Description Heavy chain variable region, E26 variants; Description No. 182:Description Heavy chain variable region, human IgG1 subgroup III; Description No. 183-Description Heavy chain variable region, humanized, immunoglobulin; Description No. 184-Description Heavy Chain Variable Region, immunosuppressant; Description No. 185-Description Heavy chain variable region, immunoglobulin; Description No. 186-Description Heavy chain variable region, or mature/immunoglobulin; Description No. 187-Description heavy chain variable region, variant; Description No. 188-Description Heavy chain variable region, with peptide signal; Description No. 189-Description Heavy chain variable region-CDR1; Description No. 190-Description Heavy chain variable region-CDR2; Description No. 191-Description Heavy chain variable region-CDR3; Description No. 192-Description Heavy chain variable region-CH1; Description No. 193-Description Heavy chain variable, Antibody for allergic reaction peanuts; Description No. 194-Description Heavy chain variable, Antibody for psoriasis, graft-versus-host disease (prevention), acute kidney transplant rejection; Description No. 195-Description Heavy chain variable, Antibody for rheumatoid arthritis; Description No. 196-Description Heavy chain variable, Antibody for rheumatoid arthritis, lupus nephritis etc, multiple sclerosis; Description No. 197-Description Heavy chain variant; Description No. 198-Description Heavy chain V-D-J assignment; Description No. 199-Description Heavy chain wild-type; Description No. 200-Description Heavy Chain with Flag Tag; Description No. 201-Description Heavy chain with signal peptide; Description No. 202-Description Heavy chain, ANGPT2; Description No. 203-Description Heavy chain, Antibody for acute coronary syndrome, atherosclerosis; Description No. 204-Description Heavy chain, Antibody for allergic diseases; Description No. 205-Description Heavy chain, Antibody for allergic disorders; Description No. 206-Description Heavy chain, Antibody for Allograft rejection, intravenous steroid-refractory ulcerative colitis, kidney transplantation, psoriasis; Description No. 207-Description Heavy chain, Antibody for Allograft rejection, graft-versus-host disease; Description No. 208-Description Heavy chain, Antibody for asthma, rheumatoid arthritis, leukemia, inflammatory diseases; Description No. 209-Description Heavy Chain, Antibody for Crohn's disease and rheumatoid arthritis; Description No. 210-Description Heavy chain, Antibody for Crohn's disease, psoriasis, ankylosing spondylitis; Description No. 211-Description Heavy chain, Antibody for Crohn's disease, Psoriasis, Transplantation, Type 1 diabetes, Ulcerative colitis, Multiple sclerosis, Atherosclerosis; Description No. 212-Description Heavy chain, Antibody for diabetes mellitus type 1; Description No. 213-Description Heavy chain, Antibody for diabetes mellitus type 1, psoriasis; Description No. 214-Description Heavy chain, Antibody for diabetes, vascular disease, acne, cancer and psoriasis; Description No. 215-Description Heavy chain, Antibody for Idiopathic pulmonary fibrosis; Description No. 216-Description Heavy chain, Antibody for idiopathic pulmonary fibrosis, focal segmental glomerulosclerosis, cancer; Description No. 217-Description Heavy chain, Antibody for osteoporosis; Description No. 218-Description Heavy chain, Antibody for osteoporosis, Denosumab αOPGL-1; Description No. 219-Description Heavy Chain, Antibody for paroxysmal nocturnal hemoglobinuria; Description No. 220-Description Heavy Chain, Antibody for Plaque-type psoriasis; Description No. 221-Description Heavy chain, Antibody for prevention of organ transplant rejections; Description No. 222-Description Heavy chain, Antibody for psoriasis; Description No. 223-Description Heavy chain, Antibody for psoriasis, organ transplant immunological rejection suppression; Description No. 224-Description Heavy chain, Antibody for Psoriasis, rheumatoid arthritis; Description No. 225-Description Heavy chain, Antibody for Psoriasis, rheumatoid arthritis, sciatica, lumbar radicular pain; Description No. 226-Description Heavy chain, Antibody for psoriasis, Crohn's disease, multiple sclerosis; Description No. 227-Description Heavy chain, Antibody for Psoriatic arthritis; Description No, 228-Description Heavy chain, Antibody for rheumatic diseases; Description No. 229-Description Heavy chain, Antibody for rheumatoid arthritis; Description No, 230-Description Heavy chain, Antibody for Rheumatoid arthritis, disease-modifying anti-rheumatic drug; Description No. 231-Description Heavy chain, Antibody for Rheumatoid arthritis, Multiple sclerosis; Description No. 232-Description Heavy Chain, Antibody for rheumatoid arthritis, psoriatic arthritis, ankylosing spondylitis, Crohn's disease, ulcerative colitis, moderate to severe chronic psoriasis and juvenile idiopathic arthritis, D2E7; Description No. 233-Description Heavy chain, Antibody for Systemic lupus erythematosus; Description No. 234-Description Heavy Chain, Antibody for ulcerative colitis and Crohn's disease; Description No. 235-Description Heavy chain, Anti-EGFr; Description No. 236-Description Heavy chain, anti-IGFR Fab-hLIGHT; Description No. 237-Description Heavy chain, chimeric; Description No. 238-Description Heavy chain, fusion; Description No. 239-Description Heavy chain, human subgroup II; Description No. 240-Description Heavy chain, immunoglobulin; Description No. 241-Description Heavy Chain, immunosuppressant; Description No. 242-Description Heavy chain, immunosuppressive drug; Description No. 243-Description Heavy chain, Mus musculus; Description No. 244-Description Heavy chain, VEGFA; Description No. 245-Description Heavy chain-constant and variable region; Description No. 246-Description Heavy chain-constant region; Description No. 247-Description Heavy chain-constant region of Hu1D10-IgG1; Description No. 248-Description Heavy chain-variable region; Description No. 249-Description Heavy chain-variable region of Hu1d10-IgG2M3 or Hu1D10-IgG1; Description No. 250-Description Heavy CHIMERIC chain 1, immunosuppressant, Anti-CD25 antibody; Description No. 251-Description Heavy-chain-CDR1; Description No. 252-Description Heavy-chain-CDR2; Description No. 253-Description Heavy-chain-CDR3; Description No. 254-Description Herceptin Heavy chain variable region-CH1 (Heavy chain variable region(1-120)+CH1(121-218)); Description No. 255-Description H-GAMMA-1 (Heavy chain variable region(1-118)+CH1(119-216) HINGE-REGION(217-231)+CH2(232-341)+CH3(342-448)); Description No. 256-Description H-GAMMA-1 (Heavy chain variable region(1-120)+CH1(121-218)+HINGE-REGION(219-233) CH2(234-343) CH3(344-450); Description No. 257-Description H-GAMMA-1 (Heavy chain variable region(1-121)+CHI(122-219)+HINGE-REGION(220-220)1012(221-330)+CH3(331-437); Description No. 258-Description Hinge, CH2 and CH3 domain of IgG1; Description No. 259-Description huHMFG1-scFv; Description No. 260-Description HuLuc-63 Heavy chain variable CDR1; Description No. 261-Description HuLuc-63 Heavy chain variable CDR2; Description No. 262-Description HuLuc-63 Heavy chain variable CDR3; Description No. 263-Description HuLuc-63 Light chain variable CDR1; Description No. 264-Description HuLuc-63 Light chain variable CDR2; Description No. 265-Description HuLuc-63 Light chain variable CDR3; Description No. 266-Description human Heavy chain—constant region; Description No. 267-Description Human IgG2 hinge region; Description No. 268-Description Humanized Heavy chain variable region-CHI (Heavy chain variable region(1-121) CH1(122-219)); Description No. 269-Description Humanized Heavy chain variable region-CH1(Heavy chain variable region(1-121)+CH1(122-201); Description No. 270-Description Humanized Light chain-KAPPA (V-KAPPA(1-107)+C-KAPPA(108-211)); Description No. 271-Description Humanized Light chain-KAPPA (V-KAPPA(1-107)+C-KAPPA(108-214)); Description No. 272-Description Humanized L-KAPPA; Description No. 273-Description Human-mouse chimeric anti-CD20 Heavy chain 1; Description No. 274-Description Human-mouse chimeric anti-CD20 Light chain 1; Description No. 275-Description Ig gamma-1 chain C region; Description No. 276-Description Ig kappa constant region; Description No. 277-Description IGHG1 constant region; Description No. 278-Description immunosuppressive drug; Description No. 279-Description Isoleucine zipper; Description No. 280-Description Kappa constant region; Description No. 281-Description kappa light chain; Description No. 282-Description Kappa Light Chain—variable region; Description No. 283-Description Lambda light chain; Description No. 284-Description Light chain; Description No. 285-Description Light Chain—variable region; Description No. 286-Description Light Chain (Genetic Recombination), Antibody for paroxysmal nocturnal hemoglobinuria; Description No. 287-Description Light chain (L-KAPPA (V-KAPPA(1-107)+C4KAPPA(108-214)); Description No. 288-Description Light chain 1; Description No. 289-Description Light Chain 1, Antibody for immunosuppressant; Description No. 290-Description Light chain 1, Anti-HER2; Description No. 291-Description Light chain 2; Description No. 292-Description Light chain 3; Description No. 293-Description Light chain 4; Description No. 294-Description Light chain amino acid sequence humanized; Description No. 295-Description Light chain and lambda constant region; Description No. 296-Description Light chain antigen binding region; Description No, 297-Description Light chain CDR; Description No. 298-Description Light Chain CDR 1, immunosuppressant; Description No, 299-Description Light Chain CDR 2, immunosuppressant; Description No. 300-Description Light Chain CDR 3, immunosuppressant; Description No. 301-Description Light chain CDR grafted anti-IL-5; Description No. 302-Description Light chain CDR1; Description No. 303-Description Light Chain CDR1, Antibody for paroxysmal nocturnal hemoglobinuria; Description No. 304-Description Light chain CDR1, Antibody for rheumatoid arthritis; Description No. 305-Description Light Chain CDR1, immunosuppressant; Description No. 306-Description Light chain CDR2; Description No. 307-Description Light Chain CDR2, Antibody for paroxysmal nocturnal hemoglobinuria; Description No, 308-Description Light chain CDR2, Antibody for rheumatoid arthritis; Description No. 309-Description Light Chain CDR2, immunosuppressant; Description No, 310-Description Light chain CDR3; Description No. 311-Description Light Chain CDR3, Antibody for paroxysmal nocturnal hemoglobinuria; Description No. 312-Description Light chain CDR3, Antibody for rheumatoid arthritis; Description No. 313-Description Light Chain CDR3, immunosuppressant; Description No. 314-Description Light chain chimeric; Description No. 315-Description Light chain Ck; Description No, 316-Description Light chain consensus, hum KI, light kappa subgroup I; Description No. 317-Description Light chain constant region; Description No. 318-Description Light chain constant region kappa; Description No. 319-Description Light chain constant region of Hu1D10-IgG2M3 or Hu1D10-IgG1; Description No. 320-Description Light chain constant region, kappa; Description No. 321-Description Light chain constant region, lambda, human; Description No. 322-Description Light chain D; Description No. 323-Description Light chain E; Description No. 324-Description Light chain F; Description No. 325-Description Light chain humanized construct LI I; Description No. 326-Description Light chain humanized construct L13; Description No. 327-Description Light chain humanized construct L14; Description No. 328-Description Light chain humanized construct L15; Description No, 329-Description Light chain humanized construct L16; Description No. 330-Description Light chain humanized construct L17; Description No, 331-Description Light chain humanized construct L18; Description No. 332-Description Light chain humanized construct LG; Description No. 333-Description Light chain IgG4, immunomodulator; Description No. 334-Description Light chain immunoglobulin variable region; Description No. 335-Description Light chain immunoglobulin; Description No, 336-Description Light chain kappa; Description No. 337-Description Light chain Kappa, Antibody for allergic reaction peanuts; Description No. 338-Description Light chain kappa consensus framework, human; Description No. 339-Description Light chain kappa consensus sequence; Description No. 340-Description Light chain kappa constant; Description No. 341-Description Light chain kappa constant region; Description No. 342-Description Light chain kappa sequence; Description No. 343-Description Light chain kappa variable region; Description No. 344-Description Light chain leader and variable region of the murine anti4G1F-I receptor antibody; Description No. 345-Description Light chain mature; Description No. 346-Description Light chain mature fragment; Description No. 347-Description Light chain mature immunoglobulin; Description No. 348-Description Light chain mature protein, Antibody for rheumatic diseases; Description No, 349-Description Light chain mature variable region; Description No. 350-Description Light chain of huAbF46414-A1(H36Y) and human kappa constant region; Description No. 351:Description Light chain polypeptide; Description No. 352-Description Light chain protein; Description No. 353-Description Light chain sequence; Description No. 354-Description Light chain used in humanization; Description No. 355-Description Light chain variable and constant chain; Description No. 356-Description Light chain variable domain of anti-alpha2-integrin; Description No, 357-Description Light chain variable domain of anti-alpha2-integrin mAb; Description No. 358-Description Light Chain Variable Domain, Antibody for rheumatoid arthritis, psoriatic arthritis, ankylosing spondylitis, Crohn's disease, ulcerative colitis, moderate to severe chronic psoriasis and juvenile idiopathic arthritis, D2E7; Description No. 359-Description Light chain variable domain, immunosuppressant for lupus; Description No. 360-Description Light chain variable kappa; Description No. 361-Description Light chain variable kappa, Amino acid sequence encoded by the VK gene; Description No. 362-Description Light chain variable of scFv, immunosuppressant for lupus; Description No. 363-Description Light chain variable region; Description No. 364-Description Light chain variable region; Description No, 365-Description light chain variable region (excludes the light chain variable region sequence of the ErbB3 binding site of 16F); Description No. 366-Description light chain variable region (excludes the light chain variable region sequence of the IGF-1R binding site of 16F); Description No. 367-Description light chain variable region (V_{L}); Description No. 368-Description Light chain variable region 1; Description No. 369-Description Light chain variable region 2; Description No. 370-Description Light chain variable region and human IgG1 constant region; Description No. 371-Description light chain variable region and light chain; Description No. 372-Description Light chain variable region consensus framework; Description No. 373-Description Light chain variable region domain (as translated) listed in U.S. Pat. No. 5,736,137; Description No. 374-Description Light chain variable region domain chain 1, Anti-IgE antibody; Description No. 375-Description Light chain variable region domain listed in U.S. Pat. No. 5,736,137 (with signal sequence removed); Description No. 376-Description Light chain variable region domain, Antibody for Fibrotic diseases, scarring, diffuse scleroderma; Description No. 377-Description light chain variable region dual variable domain; Description No. 378-Description Light chain variable region humanized construct L11; Description No. 379-Description Light chain variable region humanized construct L13; Description No. 380-Description Light chain variable region humanized construct L14; Description No. 381-Description Light chain variable region humanized construct L15; Description No. 382-Description Light chain variable region humanized construct L16; Description No. 383-Description Light chain variable region humanized construct LI 7; Description No. 384-Description Light chain variable region humanized construct L18; Description No. 385-Description Light chain variable region humanized construct L6; Description No. 386-Description Light chain variable region kappa; Description No. 387-Description Light chain variable region of Hu1D10-IgG2M3 or Hu1D10-IgG1; Description No. 388-Description Light chain variable region variant; Description No. 389-Description Light chain variable region with predicted signal; Description No. 390-Description Light chain variable region without predicted signal; Description No. 391-Description Light chain variable region without signal sequence; Description No. 392-Description Light chain variable region, Antibody for acute coronary syndrome, atherosclerosis; Description No. 393-Description Light chain variable region, Antibody for allograft rejection; Description No. 394-Description Light Chain Variable Region, Antibody for chronic plaque psoriasis; Description No. 395-Description Light chain variable region, Antibody for idiopathic pulmonary fibrosis, focal segmental glomerulosclerosis, cancer; Description No. 396-Description Light chain variable region, Antibody for Neuromyelitis optica and NMO Spectrum Disorder; Description No. 397-Description Light Chain Variable Region, Antibody for osteoporosis; Description No. 398-Description Light chain variable region, Antibody for psoriasis (blocks T-cell migration); Description No. 399-Description Light chain variable region, Antibody for Pulmonary Fibrosis; Description No. 400-Description Light chain variable region, Antibody for rheumatoid arthritis; Description No. 401-Description Light chain variable region, camelid derived; Description No. 402-Description Light chain variable region, chimeric; Description No. 403-Description Light chain variable region, Chimeric antigen receptor with cd19Binding domain; Description No. 404-Description Light chain variable region, E26 variants; Description No. 405-Description Light chain variable region, Human kappa; Description No. 406-Description Light chain variable region, humanized; Description No. 407-Description Light chain variable region, humanized, immunoglobulin; Description No. 408-Description Light Chain Variable Region, immunosuppressant; Description No. 409-Description Light chain variable region, immunoglobulin; Description No. 410-Description Light chain variable region, or mature/immunoglobulin; Description No. 411-Description light chain variable region, variant; Description No. 412-Description Light chain variable region; Light chain C; Description No. 413-Description Light chain variable region; Light chain D; Description No. 414-Description Light chain variable region; Light chain E; Description No. 415-Description Light chain variable region; Light chain F; Description No. 416-Description Light chain variable region-CDR1 From U.S. Pat. No. 8,557,243; Description No. 417-Description Light chain variable region-CDR2 From U.S. Pat. No. 8,557,243; Description No. 418-Description Light chain variable region-CDR3 From U.S. Pat. No. 8,557,243; Description No. 419-Description Light chain variable, Antibody for psoriasis, graft-versus-host disease (prevention), acute kidney transplant rejection; Description No. 420-Description Light chain variable, Antibody for rheumatoid arthritis; Description No. 421-Description Light chain variable, Antibody for rheumatoid arthritis, lupus nephritis etc, multiple sclerosis; Description No. 422-Description Light chain variant; Description No. 423-Description Light chain V-J assignment; Description No. 424-Description Light chain wild-type; Description No. 425-Description Light chain with signal peptide; Description No. 426-Description Light chain, 71F10Fab-hLIGHT fusion; Description No. 427-Description Light chain, ANGPT2; Description No. 428-Description Light chain, Antibody for acute coronary syndrome, atherosclerosis; Description No. 429-Description Light chain, Antibody for allergic diseases; Description No. 430-Description Light chain, Antibody for allergic disorders; Description No. 431-Description Light chain, Antibody for Allograft rejection, intravenous steroid-refractory ulcerative colitis, kidney transplantation, psoriasis; Description No. 432-Description Light chain, Antibody for Allograft rejection, graft-versus-host disease; Description No. 433-Description Light chain, Antibody for asthma, rheumatoid arthritis, leukemia, inflammatory diseases; Description No. 434-Description Light Chain, Antibody for Crohn's disease and rheumatoid arthritis; Description No. 435-Description Light chain, Antibody for Crohn's disease, psoriasis, ankylosing spondylitis; Description No. 436-Description Light chain, Antibody for Crohn's disease, Psoriasis, Transplantation, Type I diabetes, Ulcerative colitis, Multiple sclerosis, Atherosclerosis; Description No. 437-Description Light chain, Antibody for diabetes mellitus type 1, psoriasis; Description No. 438-Description Light chain, Antibody for diabetes mellitus type 2; Description No. 439-Description Light chain, Antibody for diabetes, vascular disease, acne, cancer and psoriasis; Description No. 440-Description Light chain, Antibody for Idiopathic pulmonary fibrosis; Description No. 441-Description Light chain, Antibody for idiopathic pulmonary fibrosis, focal segmental glomerulosclerosis, cancer; Description No. 442-Description Light chain, Antibody for osteoporosis; Description No. 443-Description Light chain, Antibody for osteoporosis, Denosumab αOPGL-1; Description No, 444-Description Light Chain, Antibody for paroxysmal nocturnal hemoglobinuria; Description No. 445-Description Light Chain, Antibody for Plaque-type psoriasis; Description No. 446-Description Light chain, Antibody for prevention of organ transplant rejections; Description No. 447-Description Light chain, Antibody for psoriasis; Description No. 448-Description Light chain, Antibody for psoriasis, organ transplant immunological rejection suppression; Description No. 449-Description Light chain, Antibody for Psoriasis, rheumatoid arthritis; Description No. 450-Description Light chain, Antibody for Psoriatic arthritis; Description No. 451-Description Light chain, Antibody for rheumatic diseases; Description No. 452-Description Light chain, Antibody for rheumatoid arthritis; Description No, 453-Description Light chain, Antibody for Rheumatoid arthritis, disease-modifying anti-rheumatic drug; Description No. 454-Description Light chain, Antibody for Rheumatoid arthritis, Multiple sclerosis; Description No, 455-Description Light Chain, Antibody for rheumatoid arthritis, psoriatic arthritis, ankylosing spondylitis, Crohn's disease, ulcerative colitis, moderate to severe chronic psoriasis and juvenile idiopathic arthritis, D2E7; Description No. 456-Description Light chain, Antibody for Systemic lupus erythematosus; Description No. 457-Description Light Chain, Antibody for ulcerative colitis and Crohn's disease; Description No. 458-Description Light chain, anti-CD23 Fab-hLIGHT fusion; Description No. 459-Description Light chain, chimeric; Description No. 460-Description Light chain, Chimeric (anti-alpha2-VL-IGKC-CL); Description No. 461-Description Light chain, human subgroup; Description No. 462-Description Light Chain, immunosuppressant; Description No. 463-Description Light chain, immunosuppressive drug; Description No. 464-Description Light chain, kappa constant; Lambda chain constant region; Description No. 465-Description Light chain, lambda constant; Description No. 466-Description Light chain, lambda human Ig; Description No. 467:Description Light chain, Mus musculus; Description No, 468-Description Light chain, VEGFA; Description No. 469-Description Light chain-variable region; Description No. 470-Description Light CHIMERIC chain 1, immunosuppressant, Anti-CD25 antibody; Description No. 471-Description L-KAPPA (V-KAPPA(1-107)+C-KAPPA(108-214)); Description No. 472-Description MAb17-1A gamma; Description No. 473-Description MAb17-1A kappa; Description No. 474-Description Mouse Anti-CD20 Heavy chain; Description No. 475-Description Mouse Anti-CD20 Light chain; Description No. 476-Description Nanobody; Description No. 477-Description Polypeptide; Description No. 478-Description polypeptide, Antibody for thrombotic thrombocytopenic purpura, acute coronary syndrome; Description No. 479-Description Scf Light chain variable region-Heavy; Description No. 480-Description ScFv; Description No. 481-Description scFv fusion protein; Description No. 482-Description Scfv Heavy-Light; Description No. 483-Description scFv immunosuppressant for lupus; Description No. 484-Description scFv, Antibody for allergic reaction peanuts; Description No. 485-Description ScFv, BHA10 ScFvs with S46L(V_{L}) stabilizing mutation; Description No. 486-Description ScFv, BHA10 ScFvs with V55G(V_{L}) stabilizing mutation; Description No. 487-Description Scfv, Chimeric antigen receptor with cd19Binding domain; Description No. 488-Description scFv-CH chain; Description No. 489-Description SEA/E-120; Description No. 490-Description secretory signal sequence of Heavy chain; Description No. 491-Description Single chain; Description No. 492-Description Single chain antibody; Description No. 493-Description Single chain scFv; Description No. 494-Description single chain variable fragment; Description No. 495-Description single chain variable fragment (scFv); Description No. 496-Description single chain variable region; Description No. 497-Description Single heavy chain variable domain; Description No. 498-Description Single variable domain antibody; Description No. 499-Description Single-chain fusion peptide; Description No. 500-Description single-domain; Description No. 501-Description single-domain antibody (dAb); Description No. 502-Description single-domain antibody (sdAb); Description No. 503-Description Small modular immunopharmaceutical (smip) polypeptide; Description No. 504-Description Variable domain antibody; Description No. 505-Description Variable region; Description No. 506-Description variant Fc region; Description No. 507-Description VH-VL; and Description No. 508-Description VL-VH.

In some embodiments, the payload region of the viral particle comprises one or more nucleic acid sequences encoding Pfiliximab, a fragment or variant thereof. As a non-limiting example, the payload region of the viral particle comprises one or more nucleic acid sequences encoding Pfiliximab may be used to treat, prevent and/or reduce the effects of multiple sclerosis. As another non-limiting example, the payload region of the viral particle comprises one or more nucleic acid sequences encoding Priliximab, a fragment or variant thereof may be used to treat, prevent and/or reduce the effects of Crohns Disease,

In some embodiments, the payload region of the viral particle comprises one or more nucleic acid sequences encoding Rovelizumab, a fragment or variant thereof. As a non-limiting example, the payload region of the viral particle comprises one or more nucleic acid sequences encoding Rovelizumab, a fragment or variant thereof may be used to treat, prevent and/or reduce the effects of multiple sclerosis.

In some embodiments, the payload region of the viral particle comprises one or more nucleic acid sequences encoding Nerelimomab, a fragment or variant thereof. As a non-limiting example, the payload region of the viral particle comprises one or more nucleic acid sequences encoding Nerelimomab, a fragment or variant thereof may be used as an immunosuppressant.

In some embodiments, the payload region of the viral particle comprises one or more nucleic acid sequences encoding BAYX1351, a fragment or variant thereof. As a non-limiting example, the payload region of the viral particle comprises one or more nucleic acid sequences encoding BAYX1351, a fragment or variant thereof may be used as an immunosuppressant.

In some embodiments, the payload region of the viral particle comprises one or more nucleic acid sequences encoding Clenoliximab (also known as CE9γ4PE, MEC-151 and PRIMATIZED®), a fragment or variant thereof. As a non-limiting example, the payload region of the viral particle comprises one or more nucleic acid sequences encoding Clenoliximab (also known as CE9γ4PE, IDEC-151 and PRIMATIZED®), a fragment or variant thereof may be used to treat, prevent or reduce the effects of rheumatoid arthritis and/or asthma. As a non-limiting example, the payload region of the viral particle comprises one or more nucleic acid sequences encoding the heavy chain of Clenoliximab (also known as CE9γ4PE, IDEC-151 and PRIMATIZED®), a fragment or variant thereof may be used to treat, prevent or reduce the effects of rheumatoid arthritis and/or asthma. As a non-limiting example, the payload region of the viral particle comprises one or more nucleic acid sequences encoding the light chain of Clenoliximab (also known as CE9γ4PE, IDEC-151 and PRIMATIZED®), a fragment or variant thereof may be used to treat, prevent or reduce the effects of rheumatoid arthritis and/or asthma. As a non-limiting example, the payload region of the viral particle comprises one or more nucleic acid sequences encoding the heavy chain of Clenoliximab (also known as CE9γ4PE, IDEC-151 and PRIMATIZED®) as described in U.S. Pat. No. 6,136,310 as SEQ ID NO: 11 (the contents of which are herein incorporated by reference in its entirety), a fragment or variant thereof may be used to treat, prevent or reduce the effects of rheumatoid arthritis and/or asthma. As a non-limiting example, the payload region of the viral particle comprises one or more nucleic acid sequences encoding the light chain of Clenoliximab (also known as CE974PE, IDEC-151 and PRIMATIZED®) as described in U.S. Pat. No. 6,136,310 as SEC) ID NO: 5 (the contents of which are herein incorporated by reference in its entirety), a fragment or variant thereof may be used to treat, prevent or reduce the effects of rheumatoid arthritis and/or asthma.

In some embodiments, the payload region of the viral particle comprises one or more nucleic acid sequences encoding Maslimomab, a fragment or variant thereof. As a non-limiting example, the payload region of the viral particle comprises one or more nucleic acid sequences encoding Maslimomab, a fragment or variant thereof may be used as an immunosuppressant.

In some embodiments, the payload region of the viral particle comprises one or more nucleic acid sequences encoding Atorolimumab (also known as P3x22914G4), a fragment or variant thereof. As a non-limiting example, the payload region of the viral particle comprises one or more nucleic acid sequences encoding Atorolimumab (also known as P3x22914G4), a fragment or variant thereof may be used as an immunosuppressant.

In some embodiments, the payload region of the viral particle comprises one or more nucleic acid sequences encoding Vapaliximab (also known as 2D10), a fragment or variant thereof. As a non-limiting example, the payload region of the viral particle comprises one or more nucleic acid sequences encoding Vapaliximab (also known as 2D10), a fragment or valiant thereof may be used as an immunosuppressant.

In some embodiments, the payload region of the viral particle comprises one or more nucleic acid sequences encoding Ziralimumab (also known as ABX-RB2, cem2.6), a fragment or variant thereof. As a non-limiting example, the payload region of the viral particle comprises one or more nucleic acid sequences encoding Ziralimumab (also known as ABX-RB2, cem2.6), a fragment or variant thereof may be used to treat, prevent and/or reduce the effects of cancer, inflammation and/or immune system disorders.

In some embodiments, the payload region of the viral particle comprises one or more nucleic acid sequences encoding Zolimomab aritox (also known as H65-ricin A chain immunotoxin and H65-RTA), a fragment or variant thereof. As a non-limiting example, the payload region of the viral particle comprises one or more nucleic acid sequences encoding Zolimomab aritox (also known as H65-ricin A chain immunotoxin and E165-RTA), a fragment or variant thereof may be used to treat, prevent or reduce the effects of systemic lupus erythematosus, graft-versus-host disease and/or cutaneous T cell lymphoma.

In some embodiments, the payload region of the viral particle comprises one or more nucleic acid sequences encoding Zanolimumab (also known as HuMax-CD4), a fragment or variant thereof. As a non-limiting example, the payload region of the viral particle comprises one or more nucleic acid sequences encoding Zanolimumab (also known as HuMax-CD4), a fragment or variant thereof may be used to treat, prevent or reduce the effects of rheumatoid arthritis, psoriasis and/or T-cell lymphoma.

In some embodiments, the payload region of the viral particle comprises one or more nucleic acid sequences encoding Bertilimumab (also known as CA17-213), a fragment or variant thereof. As a non-limiting example, the payload region of the viral particle comprises one or more nucleic acid sequences encoding Bertilimumab (also known as CA717-213), a fragment or variant thereof may be used to treat, prevent or reduce the effects of allergies.

In some embodiments, the payload region of the viral particle comprises one or more nucleic acid sequences encoding Pascolizumab (also known as SB-240683), a fragment or variant thereof. As a non-limiting example, the payload region of the viral particle comprises one or more nucleic acid sequences encoding Pascolizumab (also known as SB-240683), a fragment or variant thereof may be used to treat, prevent or reduce the effects of allergies.

In some embodiments, the payload region of the viral particle comprises one or more nucleic acid sequences encoding Odulimomab (also known as afolimomab, anti-LF A1 and ANTILFA), a fragment or variant thereof. As a non-limiting example, the payload region of the viral particle comprises one or more nucleic acid sequences encoding Odulimomab (also known as afolimomab, anti-LFA1 and ANTILFA), a fragment or variant thereof may be used to treat, prevent or reduce the effects of allograft rejection.

In some embodiments, the payload region of the viral particle comprises one or more nucleic acid sequences encoding Enlimomab pegol, a fragment or variant thereof. As a non-limiting example, the payload region of the viral particle comprises one or more nucleic acid sequences encoding Enlimomab pegol, a fragment or variant thereof may be used to treat, prevent or reduce the effects of renal transplant rejection.

In some embodiments, the payload region of the viral particle comprises a nucleic acid sequence encoding an antibody or a fragment thereof as described in United States Publication Nos. US20130122003, US20150056211, US20160069US20150056211, US20160069894 or U.S. Pat. No. 7,524,496. In a non-limiting example, the antibody targets IL-6. In another non-limiting example, the antibody targets EGF.

### Migraine and Pain Antibodies

In some embodiments, the payload region of the viral particle comprises one or more nucleic acid sequences encoding one or more of the migraine and pain payload antibody polypeptides listed in Table 10 (MP1-MP564; SEQ ID NO: 19666-20229).

TABLE 10
Migraine and Pain Antibodies

| Antibody No. | Target | Description | Antibody Name | Reference Information | SEQ ID NO |
|---|---|---|---|---|---|
| MP1 | CGRP | Heavy chain | G1, cluster | U.S. Pat. No. 9,115,194 | 19666 |
| | | | headache | SEQ ID NO: 11 | |
| MP2 | CGRP | Heavy chain | 10E4 | U.S. Pat. No. 9,102,731 | 19667 |
| | | | | SEQ ID NO: 36 | |
| MP3 | CGRP | Heavy chain | 11H9 | U.S. Pat. No. 9,102,731 | 19668 |
| | | | | SEQ ID NO: 38 | |
| MP4 | CGRP | Heavy chain | 12G8 HIL | U.S. Pat. No. 9,102,731 | 19669 |
| | | | | SEQ ID NO: 39 | |
| MP5 | CGRP | Heavy chain | 13H2 | U.S. Pat. No. 9,102,731 | 19670 |
| | | | | SEQ ID NO: 40 | |
| MP6 | CGRP | Heavy chain | 32H7 | U.S. Pat. No. 9,102,731 | 19671 |
| | | | | SEQ ID NO: 41 | |
| MP7 | CGRP | Heavy chain | A | US20120294802 | 19672 |
| | | | | SEQ ID NO: 3 | |
| MP8 | CGRP | Heavy chain | Ab1 | US20120294802 | 19673 |
| | | | | SEQ ID NO: 4 | |
| MP9 | CGRP | Heavy chain | Ab10 | US20120294802 | 19674 |
| | | | | SEQ ID NO: 94 | |
| MP10 | CGRP | Heavy chain | Ab11 | US20120294802 | 19675 |
| | | | | SEQ ID NO: 104 | |
| MP11 | CGRP | Heavy chain | Ab12 | US20120294802 | 19676 |
| | | | | SEQ ID NO: 114 | |
| MP12 | CGRP | Heavy chain | Ab13 | US20120294802 | 19677 |
| | | | | SEQ ID NO: 124 | |
| MP13 | CGRP | Heavy chain | 02E7 | U.S. Pat. No. 9,102,731 | 19678 |
| | | | | SEQ ID NO: 31 | |
| MP14 | CGRP | Heavy chain | Ab14 | US20120294802 | 19679 |
| | | | | SEQ ID NO: 134 | |
| MP15 | CGRP | Heavy chain | Ab2 | US20120294802 | 19680 |
| | | | | SEQ ID NO: 14 | |
| MP16 | CGRP | Heavy chain | Ab3 | US20120294802 | 19681 |
| | | | | SEQ ID NO: 24 | |
| MP17 | CGRP | Heavy chain | Ab4 | US20120294802 | 19682 |
| | | | | SEQ ID NO: 34 | |
| MP18 | CGRP | Heavy chain | Ab5 | US20120294802 | 19683 |
| | | | | SEQ ID NO: 44 | |
| MP19 | CGRP | Heavy chain | Ab6 | US20120294802 | 19684 |
| | | | | SEQ ID NO: 54 | |
| MP20 | CGRP | Heavy chain | Ab7 | US20120294802 | 19685 |
| | | | | SEQ ID NO: 64 | |
| MP21 | CGRP | Heavy chain | Ab8 | US20120294802 | 19686 |
| | | | | SEQ ID NO: 74 | |
| MP22 | CGRP | Heavy chain | Ab9 | US20120294802 | 19687 |
| | | | | SEQ ID NO: 84 | |
| MP23 | CGRP | Heavy chain | B | US20120294802 | 19688 |
| | | | | SEQ ID NO: 13 | |
| MP24 | CGRP | Heavy chain | 01E11/04E4/09D4 | U.S. Pat. No. 9,102,731 | 19689 |
| | | | | SEQ ID NO: 29 | |
| MP25 | CGRP | Heavy chain | C | US20120294802 | 19690 |
| | | | | SEQ ID NO: 23 | |
| MP26 | CGRP | Heavy chain | D | US20120294802 | 19691 |
| | | | | SEQ ID NO: 33 | |
| MP27 | CGRP | Heavy chain | E | US20120294802 | 19692 |
| | | | | SEQ ID NO: 43 | |
| MP28 | CGRP | Heavy chain | F | US20120294802 | 19693 |
| | | | | SEQ ID NO: 53 | |
| MP29 | CGRP | Heavy chain | G | US20120294802 | 19694 |
| | | | | SEQ ID NO: 63 | |
| MP30 | CGRP | Heavy chain | H | US20120294802 | 19695 |
| | | | | SEQ ID NO: 73 | |
| MP31 | CGRP | Heavy chain | I | US20120294802 | 19696 |
| | | | | SEQ ID NO: 83 | |
| MP32 | CGRP | Heavy chain | J | US20120294802 | 19697 |
| | | | | SEQ ID NO: 93 | |
| MP33 | CGRP | Heavy chain | K | US20120294802 | 19698 |
| | | | | SEQ ID NO: 103 | |
| MP34 | CGRP | Heavy chain | L | US20120294802 | 19699 |
| | | | | SEQ ID NO: 113 | |
| MP35 | CGRP | Heavy chain | 01H7 | U.S. Pat. No. 9,102,731 | 19700 |
| | | | | SEQ ID NO: 30 | |
| MP36 | CGRP | Heavy chain | M | US20120294802 | 19701 |
| | | | | SEQ ID NO: 123 | |
| MP37 | CGRP | Heavy chain | N | US20120294802 | 19702 |
| | | | | SEQ ID NO: 133 | |
| MP38 | CGRP | Heavy chain | 03B6 | U.S. Pat. No. 9,102,731 | 19703 |
| | | | | SEQ ID NO: 32 | |
| MP39 | CGRP | Heavy chain | 03C8/05F5/12E8 | U.S. Pat. No. 9,102,731 | 19704 |
| | | | | SEQ ID NO: 33 | |
| MP40 | CGRP | Heavy chain | 04H6 | U.S. Pat. No. 9,102,731 | 19705 |
| | | | | SEQ ID NO: 34 | |
| MP41 | CGRP | Heavy chain | 09F5 | U.S. Pat. No. 9,102,731 | 19706 |
| | | | | SEQ ID NO: 35 | |
| MP42 | CGRP | Heavy chain | 11D11 | U.S. Pat. No. 9,102,731 | 19707 |
| | | | | SEQ ID NO: 37 | |
| MP43 | TrkA | Heavy chain | BXhVH1 | WO2009098238 | 19708 |
| | | | | SEQ ID NO: 1 | |
| MP44 | TrkA | Heavy chain | BXhVH2 | WO2009098238 | 19709 |
| | | | | SEQ ID NO: 2 | |
| MP45 | TrkA | Heavy chain | BXhVH3 | WO2009098238 | 19710 |
| | | | | SEQ ID NO: 3 | |
| MP46 | TrkA | Heavy chain | BXhVH4 | WO2009098238 | 19711 |
| | | | | SEQ ID NO: 4 | |
| MP47 | TrkA | Heavy chain | BXhVH5 | WO2009098238 | 19712 |
| | | | | SEQ ID NO: 5 | |
| MP48 | TrkA | Heavy chain | BXhVH5VL1 | US20150183885 | 19713 |
| | | | | SEQ ID NO: 28 | |
| MP49 | TrkA | Heavy chain | GBR | US20150183885 | 19714 |
| | | | VH5(G42E)VL1 | SEQ ID NO: 53 | |
| MP50 | TrkA | Heavy chain | GBR | US20150183885 | 19715 |
| | | | VH5(K3Q)VL1 | SEQ ID NO: 50 | |
| MP51 | TrkA | Heavy chain | GBR | US20150183885 | 19716 |
| | | | VH5(K3Q, | SEQ ID NO: 61 | |
| | | | A49S, | | |
| | | | Y50A)VL1 | | |
| MP52 | TrkA | Heavy chain | GBR | US20150183885 | 19717 |
| | | | VH5(K3Q, | SEQ ID NO: 66 | |
| | | | A49S, | | |
| | | | Y50A, P60A, | | |
| | | | T62S)VL1 | | |
| MP53 | TrkA | Heavy chain | GBR | US20150183885 | 19718 |
| | | | VH5(K3Q, | SEQ ID NO: 62 | |
| | | | P60A, | | |
| | | | T62S)VL1 | | |
| MP54 | TrkA | Heavy chain | GBR | US20150183885 | 19719 |
| | | | VH5(K3Q, | SEQ ID NO: 58 | |
| | | | T40A)VL1 | | |
| MP55 | TrkA | Heavy chain | GBR | US20150183885 | 19720 |
| | | | VH5(K3Q, | SEQ ID NO: 63 | |
| | | | T40A, | | |
| | | | P60A, T62S)VL1 | | |
| MP56 | TrkA | Heavy chain | GBR | US20150183885 | 19721 |
| | | | VH5(K3Q, | SEQ ID NO: 67 | |
| | | | T40A, R44G, | | |
| | | | A49S, Y50A, | | |
| | | | P60A, | | |
| | | | T62S)VL1 | | |
| MP57 | TrkA | Heavy chain | GBR | US20150183885 | 19722 |
| | | | VH5(K3Q, | SEQ ID NO: 68 | |
| | | | T40A, R44G, | | |
| | | | A49S, Y50A, | | |
| | | | P60A, T62S, | | |
| | | | R94K)VL1 | | |
| MP58 | TrkA | Heavy chain | GBR | US20150183885 | 19723 |
| | | | VH5(K3Q, | SEQ ID NO: 65 | |
| | | | T40A, | | |
| | | | R44G, A49S, | | |
| | | | Y50A)VL1 | | |
| MP59 | TrkA | Heavy chain | GBR | US20150183885 | 19724 |
| | | | VH5(K3Q, | SEQ ID NO: 56 | |
| | | | V37A(VL1 | | |
| MP60 | TrkA | Heavy chain | GBR | US20150183885 | 19725 |
| | | | VH5(K3Q, | SEQ ID NO: 57 | |
| | | | V37A)VL1(*) | | |
| MP61 | TrkA | Heavy chain | GBR | US20150183885 | 19726 |
| | | | VH5(K3Q, | SEQ ID NO: 60 | |
| | | | V37A, | | |
| | | | R44G)VL1 | | |
| MP62 | TrkA | Heavy chain | GBR | US20150183885 | 19727 |
| | | | VH5(K3Q, | SEQ ID NO: 64 | |
| | | | V37A, | | |
| | | | T40A, P60A, | | |
| | | | T62S)VL1 | | |
| MP63 | TrkA | Heavy chain | GBR | US20150183885 | 19728 |
| | | | VH5(P60A, | SEQ ID NO: 59 | |
| | | | T62S)VL1 | | |
| MP64 | TrkA | Heavy chain | GBR | US20150183885 | 19729 |
| | | | VH5(R94K)VL1 | SEQ ID NO: 55 | |
| MP65 | TrkA | Heavy chain | GBR | US20150183885 | 19730 |
| | | | VH5(V37A)VL1 | SEQ ID NO: 51 | |
| MP66 | TrkA | Heavy chain | GBR | US20150183885 | 19731 |
| | | | VH5(V37A)VL1(*) | SEQ ID NO: 52 | |
| MP67 | TrkA | Heavy chain | GBR | US20150183885 | 19732 |
| | | | VH5(V89L)VL1 | SEQ ID NO: 54 | |
| MP68 | TrkA | Heavy chain | HUVHWOV | WO2009098238 | 19733 |
| | | | | SEQ ID NO: 6 | |
| MP69 | TrkA | Heavy chain | mVHEP | WO2009098238 | 19734 |
| | | | | SEQ ID NO: 15 | |
| MP70 | GFRα3 | Heavy chain | H1M2236N | U.S. Pat. No. 8,968,736 | 19735 |
| | | variable region | | SEQ ID NO: 397 | |
| MP71 | GFRα3 | Heavy chain | H1M2243N | U.S. Pat. No. 8,968,736 | 19736 |
| | | variable region | | SEQ ID NO: 381 | |
| MP72 | GFRα3 | Heavy chain | H4H2207N | U.S. Pat. No. 8,968,736 | 19737 |
| | | variable region | | SEQ ID NO: 2 | |
| MP73 | GFRα3 | Heavy chain | H4H2210N | U.S. Pat. No. 8,968,736 | 19738 |
| | | variable region | | SEQ ID NO: 66 | |
| MP74 | GFRα3 | Heavy chain | H4H2212N | U.S. Pat. No. 8,968,736 | 19739 |
| | | variable region | | SEQ ID NO: 18 | |
| MP75 | GFRα3 | Heavy chain | H4H2234N | U.S. Pat. No. 8,968,736 | 19740 |
| | | variable region | | SEQ ID NO: 82 | |
| MP76 | GFRα3 | Heavy chain | H4H2236N3 | U.S. Pat. No. 8,968,736 | 19741 |
| | | variable region | | SEQ ID NO: 34 | |
| MP77 | GFRα3 | Heavy chain | H4H2243N2 | U.S. Pat. No. 8,968,736 | 19742 |
| | | variable region | | SEQ ID NO: 50 | |
| MP78 | GFRα3 | Heavy chain | H4H2291S | U.S. Pat. No. 8,968,736 | 19743 |
| | | variable region | | SEQ ID NO: 98 | |
| MP79 | GFRα3 | Heavy chain | H4H2292S | U.S. Pat. No. 8,968,736 | 19744 |
| | | variable region | | SEQ ID NO: 114 | |
| MP80 | GFRα3 | Heavy chain | H4H2293P | U.S. Pat. No. 8,968,736 | 19745 |
| | | variable region | | SEQ ID NO: 130 | |
| MP81 | GFRα3 | Heavy chain | H4H2294S | U.S. Pat. No. 8,968,736 | 19746 |
| | | variable region | | SEQ ID NO: 146 | |
| MP82 | GFRα3 | Heavy chain | H4H2295S | U.S. Pat. No. 8,968,736 | 19747 |
| | | variable region | | SEQ ID NO: 162 | |
| MP83 | GFRα3 | Heavy chain | H4H2296S | U.S. Pat. No. 8,968,736 | 19748 |
| | | variable region | | SEQ ID NO: 178 | |
| MP84 | GFRα3 | Heavy chain | H4H2341S | U.S. Pat. No. 8,968,736 | 19749 |
| | | variable region | | SEQ ID NO: 194 | |
| MP85 | GFRα3 | Heavy chain | H4H2342P | U.S. Pat. No. 8,968,736 | 19750 |
| | | variable region | | SEQ ID NO: 210 | |
| MP86 | GFRα3 | Heavy chain | H4H2344S | U.S. Pat. No. 8,968,736 | 19751 |
| | | variable region | | SEQ ID NO: 226 | |
| MP87 | GFRα3 | Heavy chain | H4H2345S | U.S. Pat. No. 8,968,736 | 19752 |
| | | variable region | | SEQ ID NO: 242 | |
| MP88 | GFRα3 | Heavy chain | H4H2346S | U.S. Pat. No. 8,968,736 | 19753 |
| | | variable region | | SEQ ID NO: 258 | |
| MP89 | GFRα3 | Heavy chain | H4H2352S | U.S. Pat. No. 8,968,736 | 19754 |
| | | variable region | | SEQ ID NO: 290 | |
| MP90 | GFRα3 | Heavy chain | H4H2354S | U.S. Pat. No. 8,968,736 | 19755 |
| | | variable region | | SEQ ID NO: 306 | |
| MP91 | GFRα3 | Heavy chain | H4H2355S | U.S. Pat. No. 8,968,736 | 19756 |
| | | variable region | | SEQ ID NO: 322 | |
| MP92 | GFRα3 | Heavy chain | H4H2357S | U.S. Pat. No. 8,968,736 | 19757 |
| | | variable region | | SEQ ID NO: 338 | |
| MP93 | GFRα3 | Heavy chain | H4H2364S | U.S. Pat. No. 8,968,736 | 19758 |
| | | variable region | | SEQ ID NO: 354 | |
| MP94 | hNav1.7 | Heavy chain | H1H1015B | WO2014159595 | 19759 |
| | | variable region | | SEQ ID NO: 126 | |
| MP95 | hNav1.7 | Heavy chain | H1H1019B | WO2014159595 | 19760 |
| | | variable region | | SEQ ID NO: 110 | |
| MP96 | hNav1.7 | Heavy chain | H1H1021B | WO2014159595 | 19761 |
| | | variable region | | SEQ ID NO: 428 | |
| MP97 | hNav1.7 | Heavy chain | H1H1022B | WO2014159595 | 19762 |
| | | variable region | | SEQ ID NO: 130 | |
| MP98 | hNav1.7 | Heavy chain | H1H1023B | WO2014159595 | 19763 |
| | | variable region | | SEQ ID NO: 134 | |
| MP99 | hNav1.7 | Heavy chain | H1H1026B | WO2014159595 | 19764 |
| | | variable region | | SEQ ID NO: 138 | |
| MP100 | hNav1.7 | Heavy chain | H1H1028B | WO2014159595 | 19765 |
| | | variable region | | SEQ ID NO: 430 | |
| MP101 | hNav1.7 | Heavy chain | H1H1029B | WO2014159595 | 19766 |
| | | variable region | | SEQ ID NO: 432 | |
| MP102 | hNav1.7 | Heavy chain | H1H1030B | WO2014159595 | 19767 |
| | | variable region | | SEQ ID NO: 142 | |
| MP103 | hNav1.7 | Heavy chain | H1H1032B | WO2014159595 | 19768 |
| | | variable region | | SEQ ID NO: 146 | |
| MP104 | hNav1.7 | Heavy chain | H1H1036B | WO2014159595 | 19769 |
| | | variable region | | SEQ ID NO: 434 | |
| MP105 | hNav1.7 | Heavy chain | H1H1038B | WO2014159595 | 19770 |
| | | variable region | | SEQ ID NO: 150 | |
| MP106 | hNav1.7 | Heavy chain | H1H1039B | WO2014159595 | 19771 |
| | | variable region | | SEQ ID NO: 436 | |
| MP107 | hNav1.7 | Heavy chain | H1H1040B | WO2014159595 | 19772 |
| | | variable region | | SEQ ID NO: 438 | |
| MP108 | hNav1.7 | Heavy chain | H1H1041B | WO2014159595 | 19773 |
| | | variable region | | SEQ ID NO: 154 | |
| MP109 | hNav1.7 | Heavy chain | H1H1042B | WO2014159595 | 19774 |
| | | variable region | | SEQ ID NO: 440 | |
| MP110 | hNav1.7 | Heavy chain | H1H1044B | WO2014159595 | 19775 |
| | | variable region | | SEQ ID NO: 158 | |
| MP111 | hNav1.7 | Heavy chain | H1H1045B | WO2014159595 | 19776 |
| | | variable region | | SEQ ID NO: 162 | |
| MP112 | hNav1.7 | Heavy chain | H1H1050B | WO2014159595 | 19777 |
| | | variable region | | SEQ ID NO: 166 | |
| MP113 | hNav1.7 | Heavy chain | H1H1052B | WO2014159595 | 19778 |
| | | variable region | | SEQ ID NO: 442 | |
| MP114 | hNav1.7 | Heavy chain | H1H1055B | WO2014159595 | 19779 |
| | | variable region | | SEQ ID NO: 170 | |
| MP115 | hNav1.7 | Heavy chain | H1H1056B | WO2014159595 | 19780 |
| | | variable region | | SEQ ID NO: 174 | |
| MP116 | hNav1.7 | Heavy chain | H1H1058B | WO2014159595 | 19781 |
| | | variable region | | SEQ ID NO: 444 | |
| MP117 | hNav1.7 | Heavy chain | H1H1059B | WO2014159595 | 19782 |
| | | variable region | | SEQ ID NO: 178 | |
| MP118 | hNav1.7 | Heavy chain | H1H1060B | WO2014159595 | 19783 |
| | | variable region | | SEQ ID NO: 182 | |
| MP119 | hNav1.7 | Heavy chain | H1H1061B | WO2014159595 | 19784 |
| | | variable region | | SEQ ID NO: 446 | |
| MP120 | hNav1.7 | Heavy chain | H1H1065B | WO2014159595 | 19785 |
| | | variable region | | SEQ ID NO: 448 | |
| MP121 | hNav1.7 | Heavy chain | H1H1066B | WO2014159595 | 19786 |
| | | variable region | | SEQ ID NO: 450 | |
| MP122 | hNav1.7 | Heavy chain | H1H1067B | WO2014159595 | 19787 |
| | | variable region | | SEQ ID NO: 452 | |
| MP123 | hNav1.7 | Heavy chain | H1H1068B | WO2014159595 | 19788 |
| | | variable region | | SEQ ID NO: 454 | |
| MP124 | hNav1.7 | Heavy chain | H1H1076B | WO2014159595 | 19789 |
| | | variable region | | SEQ ID NO: 456 | |
| MP125 | hNav1.7 | Heavy chain | H1H1089B | WO2014159595 | 19790 |
| | | variable region | | SEQ ID NO: 458 | |
| MP126 | hNav1.7 | Heavy chain | H1H1090B | WO2014159595 | 19791 |
| | | variable region | | SEQ ID NO: 460 | |
| MP127 | hNav1.7 | Heavy chain | H1H1097B | WO2014159595 | 19792 |
| | | variable region | | SEQ ID NO: 462 | |
| MP128 | hNav1.7 | Heavy chain | H1H1100B | WO2014159595 | 19793 |
| | | variable region | | SEQ ID NO: 464 | |
| MP129 | hNav1.7 | Heavy chain | H1H1102B | WO2014159595 | 19794 |
| | | variable region | | SEQ ID NO: 466 | |
| MP130 | hNav1.7 | Heavy chain | H1H1106B | WO2014159595 | 19795 |
| | | variable region | | SEQ ID NO: 468 | |
| MP131 | hNav1.7 | Heavy chain | H1H1107B | WO2014159595 | 19796 |
| | | variable region | | SEQ ID NO: 470 | |
| MP132 | hNav1.7 | Heavy chain | H1H1108B | WO2014159595 | 19797 |
| | | variable region | | SEQ ID NO: 472 | |
| MP133 | hNav1.7 | Heavy chain | H1H1109B | WO2014159595 | 19798 |
| | | variable region | | SEQ ID NO: 474 | |
| MP134 | hNav1.7 | Heavy chain | H1H1111B | WO2014159595 | 19799 |
| | | variable region | | SEQ ID NO: 476 | |
| MP135 | hNav1.7 | Heavy chain | H1H1114B | WO2014159595 | 19800 |
| | | variable region | | SEQ ID NO: 426 | |
| MP136 | hNav1.7 | Heavy chain | H1H1117B | WO2014159595 | 19801 |
| | | variable region | | SEQ ID NO: 478 | |
| MP137 | hNav1.7 | Heavy chain | H1H1118B | WO2014159595 | 19802 |
| | | variable region | | SEQ ID NO: 480 | |
| MP138 | hNav1.7 | Heavy chain | H1H1119B | WO2014159595 | 19803 |
| | | variable region | | SEQ ID NO: 482 | |
| MP139 | hNav1.7 | Heavy chain | H1H1121B | WO2014159595 | 19804 |
| | | variable region | | SEQ ID NO: 484 | |
| MP140 | hNav1.7 | Heavy chain | H1H1126B | WO2014159595 | 19805 |
| | | variable region | | SEQ ID NO: 486 | |
| MP141 | hNav1.7 | Heavy chain | H1H1130B | WO2014159595 | 19806 |
| | | variable region | | SEQ ID NO: 488 | |
| MP142 | hNav1.7 | Heavy chain | H1H1131B | WO2014159595 | 19807 |
| | | variable region | | SEQ ID NO: 490 | |
| MP143 | hNav1.7 | Heavy chain | H1H1133B | WO2014159595 | 19808 |
| | | variable region | | SEQ ID NO: 492 | |
| MP144 | hNav1.7 | Heavy chain | H1H1134B | WO2014159595 | 19809 |
| | | variable region | | SEQ ID NO: 494 | |
| MP145 | hNav1.7 | Heavy chain | H1H1135B | WO2014159595 | 19810 |
| | | variable region | | SEQ ID NO: 496 | |
| MP146 | hNav1.7 | Heavy chain | H1H1137B | WO2014159595 | 19811 |
| | | variable region | | SEQ ID NO: 498 | |
| MP147 | hNav1.7 | Heavy chain | H1H1139B | WO2014159595 | 19812 |
| | | variable region | | SEQ ID NO: 500 | |
| MP148 | hNav1.7 | Heavy chain | H1H1141B | WO2014159595 | 19813 |
| | | variable region | | SEQ ID NO: 502 | |
| MP149 | hNav1.7 | Heavy chain | H1H1149B | WO2014159595 | 19814 |
| | | variable region | | SEQ ID NO: 504 | |
| MP150 | hNav1.7 | Heavy chain | H1H1153B | WO2014159595 | 19815 |
| | | variable region | | SEQ ID NO: 506 | |
| MP151 | hNav1.7 | Heavy chain | H1H1156B | WO2014159595 | 19816 |
| | | variable region | | SEQ ID NO: 508 | |
| MP152 | hNav1.7 | Heavy chain | H1H1157B | WO2014159595 | 19817 |
| | | variable region | | SEQ ID NO: 510 | |
| MP153 | hNav1.7 | Heavy chain | H1H1158B | WO2014159595 | 19818 |
| | | variable region | | SEQ ID NO: 512 | |
| MP154 | hNav1.7 | Heavy chain | H1H1162B | WO2014159595 | 19819 |
| | | variable region | | SEQ ID NO: 514 | |
| MP155 | hNav1.7 | Heavy chain | H1H1172B | WO2014159595 | 19820 |
| | | variable region | | SEQ ID NO: 516 | |
| MP156 | hNav1.7 | Heavy chain | H2M799N | WO2014159595 | 19821 |
| | | variable region | | SEQ ID NO: 823 | |
| MP157 | hNav1.7 | Heavy chain | H4H1003P | WO2014159595 | 19822 |
| | | variable region | | SEQ ID NO: 860 | |
| MP158 | hNav1.7 | Heavy chain | H4H1025P | WO2014159595 | 19823 |
| | | variable region | | SEQ ID NO: 872 | |
| MP159 | hNav1.7 | Heavy chain | H4H361 B | WO2014159595 | 19824 |
| | | variable region | | SEQ ID NO: 234 | |
| MP160 | hNav1.7 | Heavy chain | H4H362B | WO2014159595 | 19825 |
| | | variable region | | SEQ ID NO: 30 | |
| MP161 | hNav1.7 | Heavy chain | H4H362P | WO2014159595 | 19826 |
| | | variable region | | SEQ ID NO: 868 | |
| MP162 | hNav1.7 | Heavy chain | H4H365B | WO2014159595 | 19827 |
| | | variable region | | SEQ ID NO: 238 | |
| MP163 | hNav1.7 | Heavy chain | H4H367B | WO2014159595 | 19828 |
| | | variable region | | SEQ ID NO: 34 | |
| MP164 | hNav1.7 | Heavy chain | H4H368B | WO2014159595 | 19829 |
| | | variable region | | SEQ ID NO: 38 | |
| MP165 | hNav1.7 | Heavy chain | H4H370B | WO2014159595 | 19830 |
| | | variable region | | SEQ ID NO: 518 | |
| MP166 | hNav1.7 | Heavy chain | H4H371 B | WO2014159595 | 19831 |
| | | variable region | | SEQ ID NO: 242 | |
| MP167 | hNav1.7 | Heavy chain | H4H372B | WO2014159595 | 19832 |
| | | variable region | | SEQ ID NO: 246 | |
| MP168 | hNav1.7 | Heavy chain | H4H373B | WO2014159595 | 19833 |
| | | variable region | | SEQ ID NO: 250 | |
| MP169 | hNav1.7 | Heavy chain | H4H378B | WO2014159595 | 19834 |
| | | variable region | | SEQ ID NO: 520 | |
| MP170 | hNav1.7 | Heavy chain | H4H379B | WO2014159595 | 19835 |
| | | variable region | | SEQ ID NO: 254 | |
| MP171 | hNav1.7 | Heavy chain | H4H381 B | WO2014159595 | 19836 |
| | | variable region | | SEQ ID NO: 258 | |
| MP172 | hNav1.7 | Heavy chain | H4H382B | WO2014159595 | 19837 |
| | | variable region | | SEQ ID NO: 42 | |
| MP173 | hNav1.7 | Heavy chain | H4H383B | WO2014159595 | 19838 |
| | | variable region | | SEQ ID NO: 522 | |
| MP174 | hNav1.7 | Heavy chain | H4H385B | WO2014159595 | 19839 |
| | | variable region | | SEQ ID NO: 262 | |
| MP175 | hNav1.7 | Heavy chain | H4H385B | WO2014159595 | 19840 |
| | | variable region | | SEQ ID NO: 681 | |
| MP176 | hNav1.7 | Heavy chain | H4H388B | WO2014159595 | 19841 |
| | | variable region | | SEQ ID NO: 266 | |
| MP177 | hNav1.7 | Heavy chain | H4H389B | WO2014159595 | 19842 |
| | | variable region | | SEQ ID NO: 524 | |
| MP178 | hNav1.7 | Heavy chain | H4H391B | WO2014159595 | 19843 |
| | | variable region | | SEQ ID NO: 46 | |
| MP179 | hNav1.7 | Heavy chain | H4H391P | WO2014159595 | 19844 |
| | | variable region | | SEQ ID NO: 50 | |
| MP180 | hNav1.7 | Heavy chain | H4H395B | WO2014159595 | 19845 |
| | | variable region | | SEQ ID NO: 685 | |
| MP181 | hNav1.7 | Heavy chain | H4H396B | WO2014159595 | 19846 |
| | | variable region | | SEQ ID NO: 270 | |
| MP182 | hNav1.7 | Heavy chain | H4H397B | WO2014159595 | 19847 |
| | | variable region | | SEQ ID NO: 54 | |
| MP183 | hNav1.7 | Heavy chain | H4H398B | WO2014159595 | 19848 |
| | | variable region | | SEQ ID NO: 274 | |
| MP184 | hNav1.7 | Heavy chain | H4H399B | WO2014159595 | 19849 |
| | | variable region | | SEQ ID NO: 278 | |
| MP185 | hNav1.7 | Heavy chain | H4H400B | WO2014159595 | 19850 |
| | | variable region | | SEQ ID NO: 282 | |
| MP186 | hNav1.7 | Heavy chain | H4H402B | WO2014159595 | 19851 |
| | | variable region | | SEQ ID NO: 286 | |
| MP187 | hNav1.7 | Heavy chain | H4H405B | WO2014159595 | 19852 |
| | | variable region | | SEQ ID NO: 526 | |
| MP188 | hNav1.7 | Heavy chain | H4H407B | WO2014159595 | 19853 |
| | | variable region | | SEQ ID NO: 528 | |
| MP189 | hNav1.7 | Heavy chain | H4H408B | WO2014159595 | 19854 |
| | | variable region | | SEQ ID NO: 58 | |
| MP190 | hNav1.7 | Heavy chain | H4H409B | WO2014159595 | 19855 |
| | | variable region | | SEQ ID NO: 290 | |
| MP191 | hNav1.7 | Heavy chain | H4H413B | WO2014159595 | 19856 |
| | | variable region | | SEQ ID NO: 530 | |
| MP192 | hNav1.7 | Heavy chain | H4H415B | WO2014159595 | 19857 |
| | | variable region | | SEQ ID NO: 294 | |
| MP193 | hNav1.7 | Heavy chain | H4H416B | WO2014159595 | 19858 |
| | | variable region | | SEQ ID NO: 298 | |
| MP194 | hNav1.7 | Heavy chain | H4H419B | WO2014159595 | 19859 |
| | | variable region | | SEQ ID NO: 302 | |
| MP195 | hNav1.7 | Heavy chain | H4H422B | WO2014159595 | 19860 |
| | | variable region | | SEQ ID NO: 306 | |
| MP196 | hNav1.7 | Heavy chain | H4H426B | WO2014159595 | 19861 |
| | | variable region | | SEQ ID NO: 62 | |
| MP197 | hNav1.7 | Heavy chain | H4H427B | WO2014159595 | 19862 |
| | | variable region | | SEQ ID NO: 532 | |
| MP198 | hNav1.7 | Heavy chain | H4H432B | WO2014159595 | 19863 |
| | | variable region | | SEQ ID NO: 534 | |
| MP199 | hNav1.7 | Heavy chain | H4H434B | WO2014159595 | 19864 |
| | | variable region | | SEQ ID NO: 310 | |
| MP200 | hNav1.7 | Heavy chain | H4H434B | WO2014159595 | 19865 |
| | | variable region | | SEQ ID NO: 689 | |
| MP201 | hNav1.7 | Heavy chain | H4H434P | WO2014159595 | 19866 |
| | | variable region | | SEQ ID NO: 693 | |
| MP202 | hNav1.7 | Heavy chain | H4H436B | WO2014159595 | 19867 |
| | | variable region | | SEQ ID NO: 536 | |
| MP203 | hNav1.7 | Heavy chain | H4H437B | WO2014159595 | 19868 |
| | | variable region | | SEQ ID NO: 538 | |
| MP204 | hNav1.7 | Heavy chain | H4H438B | WO2014159595 | 19869 |
| | | variable region | | SEQ ID NO: 314 | |
| MP205 | hNav1.7 | Heavy chain | H4H438B | WO2014159595 | 19870 |
| | | variable region | | SEQ ID NO: 697 | |
| MP206 | hNav1.7 | Heavy chain | H4H439B | WO2014159595 | 19871 |
| | | variable region | | SEQ ID NO: 66 | |
| MP207 | hNav1.7 | Heavy chain | H4H439P | WO2014159595 | 19872 |
| | | variable region | | SEQ ID NO: 70 | |
| MP208 | hNav1.7 | Heavy chain | H4H441 B | WO2014159595 | 19873 |
| | | variable region | | SEQ ID NO: 701 | |
| MP209 | hNav1.7 | Heavy chain | H4H441 P | WO2014159595 | 19874 |
| | | variable region | | SEQ ID NO: 864 | |
| MP210 | hNav1.7 | Heavy chain | H4H442B | WO2014159595 | 19875 |
| | | variable region | | SEQ ID NO: 318 | |
| MP211 | hNav1.7 | Heavy chain | H4H443B | WO2014159595 | 19876 |
| | | variable region | | SEQ ID NO: 74 | |
| MP212 | hNav1.7 | Heavy chain | H4H444B | WO2014159595 | 19877 |
| | | variable region | | SEQ ID NO: 322 | |
| MP213 | hNav1.7 | Heavy chain | H4H445B | WO2014159595 | 19878 |
| | | variable region | | SEQ ID NO: 540 | |
| MP214 | hNav1.7 | Heavy chain | H4H446B | WO2014159595 | 19879 |
| | | variable region | | SEQ ID NO: 326 | |
| MP215 | hNav1.7 | Heavy chain | H4H448B | WO2014159595 | 19880 |
| | | variable region | | SEQ ID NO: 78 | |
| MP216 | hNav1.7 | Heavy chain | H4H453B | WO2014159595 | 19881 |
| | | variable region | | SEQ ID NO: 542 | |
| MP217 | hNav1.7 | Heavy chain | H4H456B | WO2014159595 | 19882 |
| | | variable region | | SEQ ID NO: 330 | |
| MP218 | hNav1.7 | Heavy chain | H4H457B | WO2014159595 | 19883 |
| | | variable region | | SEQ ID NO: 334 | |
| MP219 | hNav1.7 | Heavy chain | H4H458B | WO2014159595 | 19884 |
| | | variable region | | SEQ ID NO: 338 | |
| MP220 | hNav1.7 | Heavy chain | H4H460B | WO2014159595 | 19885 |
| | | variable region | | SEQ ID NO: 342 | |
| MP221 | hNav1.7 | Heavy chain | H4H461 B | WO2014159595 | 19886 |
| | | variable region | | SEQ ID NO: 346 | |
| MP222 | hNav1.7 | Heavy chain | H4H462B | WO2014159595 | 19887 |
| | | variable region | | SEQ ID NO: 350 | |
| MP223 | hNav1.7 | Heavy chain | H4H463B | WO2014159595 | 19888 |
| | | variable region | | SEQ ID NO: 354 | |
| MP224 | hNav1.7 | Heavy chain | H4H464B | WO2014159595 | 19889 |
| | | variable region | | SEQ ID NO: 358 | |
| MP225 | hNav1.7 | Heavy chain | H4H465B | WO2014159595 | 19890 |
| | | variable region | | SEQ ID NO: 362 | |
| MP226 | hNav1.7 | Heavy chain | H4H466B | WO2014159595 | 19891 |
| | | variable region | | SEQ ID NO: 366 | |
| MP227 | hNav1.7 | Heavy chain | H4H467B | WO2014159595 | 19892 |
| | | variable region | | SEQ ID NO: 370 | |
| MP228 | hNav1.7 | Heavy chain | H4H468B | WO2014159595 | 19893 |
| | | variable region | | SEQ ID NO: 82 | |
| MP229 | hNav1.7 | Heavy chain | H4H468P | WO2014159595 | 19894 |
| | | variable region | | SEQ ID NO: 86 | |
| MP230 | hNav1.7 | Heavy chain | H4H471B | WO2014159595 | 19895 |
| | | variable region | | SEQ ID NO: 90 | |
| MP231 | hNav1.7 | Heavy chain | H4H471P | WO2014159595 | 19896 |
| | | variable region | | SEQ ID NO: 94 | |
| MP232 | hNav1.7 | Heavy chain | H4H472B | WO2014159595 | 19897 |
| | | variable region | | SEQ ID NO: 374 | |
| MP233 | hNav1.7 | Heavy chain | H4H473B | WO2014159595 | 19898 |
| | | variable region | | SEQ ID NO: 378 | |
| MP234 | hNav1.7 | Heavy chain | H4H475B | WO2014159595 | 19899 |
| | | variable region | | SEQ ID NO: 382 | |
| MP235 | hNav1.7 | Heavy chain | H4H477B | WO2014159595 | 19900 |
| | | variable region | | SEQ ID NO: 386 | |
| MP236 | hNav1.7 | Heavy chain | H4H478B | WO2014159595 | 19901 |
| | | variable region | | SEQ ID NO: 544 | |
| MP237 | hNav1.7 | Heavy chain | H4H480B | WO2014159595 | 19902 |
| | | variable region | | SEQ ID NO: 390 | |
| MP238 | hNav1.7 | Heavy chain | H4H481 B | WO2014159595 | 19903 |
| | | variable region | | SEQ ID NO: 394 | |
| MP239 | hNav1.7 | Heavy chain | H4H482B | WO2014159595 | 19904 |
| | | variable region | | SEQ ID NO: 398 | |
| MP240 | hNav1.7 | Heavy chain | H4H483B | WO2014159595 | 19905 |
| | | variable region | | SEQ ID NO: 402 | |
| MP241 | hNav1.7 | Heavy chain | H4H484B | WO2014159595 | 19906 |
| | | variable region | | SEQ ID NO: 406 | |
| MP242 | hNav1.7 | Heavy chain | H4H486B | WO2014159595 | 19907 |
| | | variable region | | SEQ ID NO: 410 | |
| MP243 | hNav1.7 | Heavy chain | H4H488B | WO2014159595 | 19908 |
| | | variable region | | SEQ ID NO: 414 | |
| MP244 | hNav1.7 | Heavy chain | H4H489B | WO2014159595 | 19909 |
| | | variable region | | SEQ ID NO: 418 | |
| MP245 | hNav1.7 | Heavy chain | H4H490B | WO2014159595 | 19910 |
| | | variable region | | SEQ ID NO: 546 | |
| MP246 | hNav1.7 | Heavy chain | H4H491B | WO2014159595 | 19911 |
| | | variable region | | SEQ ID NO: 422 | |
| MP247 | hNav1.7 | Heavy chain | H1 H1 105B | WO2014159595 | 19912 |
| | | variable region | | SEQ ID NO: 198 | |
| MP248 | hNav1.7 | Heavy chain | H1 H1 123B | WO2014159595 | 19913 |
| | | variable region | | SEQ ID NO: 202 | |
| MP249 | hNav1.7 | Heavy chain | H1 H1 138B | WO2014159595 | 19914 |
| | | variable region | | SEQ ID NO: 206 | |
| MP250 | hNav1.7 | Heavy chain | H1 H1 144B | WO2014159595 | 19915 |
| | | variable region | | SEQ ID NO: 210 | |
| MP251 | hNav1.7 | Heavy chain | H1 H1 147B | WO2014159595 | 19916 |
| | | variable region | | SEQ ID NO: 214 | |
| MP252 | hNav1.7 | Heavy chain | H1 H1 155B | WO2014159595 | 19917 |
| | | variable region | | SEQ ID NO: 218 | |
| MP253 | hNav1.7 | Heavy chain | H1 H1 164B | WO2014159595 | 19918 |
| | | variable region | | SEQ ID NO: 222 | |
| MP254 | hNav1.7 | Heavy chain | H1 H1 166B | WO2014159595 | 19919 |
| | | variable region | | SEQ ID NO: 226 | |
| MP255 | hNav1.7 | Heavy chain | H1 H1 169B | WO2014159595 | 19920 |
| | | variable region | | SEQ ID NO: 230 | |
| MP256 | hNav1.7 | Heavy chain | H1 H1006P | WO2014159595 | 19921 |
| | | variable region | | SEQ ID NO: 705 | |
| MP257 | hNav1.7 | Heavy chain | H1 H1025B | WO2014159595 | 19922 |
| | | variable region | | SEQ ID NO: 722 | |
| MP258 | hNav1.7 | Heavy chain | H1 H1068B | WO2014159595 | 19923 |
| | | variable region | | SEQ ID NO: 709 | |
| MP259 | hNav1.7 | Heavy chain | H1 H1069B | WO2014159595 | 19924 |
| | | variable region | | SEQ ID NO: 186 | |
| MP260 | hNav1.7 | Heavy chain | H1 H1082B | WO2014159595 | 19925 |
| | | variable region | | SEQ ID NO: 190 | |
| MP261 | hNav1.7 | Heavy chain | H1 H1098B | WO2014159595 | 19926 |
| | | variable region | | SEQ ID NO: 194 | |
| MP262 | hNav1.7 | Heavy chain | H1 M683N | WO2014159595 | 19927 |
| | | variable region | | SEQ ID NO: 2 | |
| MP263 | hNav1.7 | Heavy chain | H1 M797N | WO2014159595 | 19928 |
| | | variable region | | SEQ ID NO: 6 | |
| MP264 | hNav1.7 | Heavy chain | H1 M799N | WO2014159595 | 19929 |
| | | variable region | | SEQ ID NO: 26 | |
| MP265 | hNav1.7 | Heavy chain | H1 M801 N | WO2014159595 | 19930 |
| | | variable region | | SEQ ID NO: 727 | |
| MP266 | hNav1.7 | Heavy chain | H1 M826N | WO2014159595 | 19931 |
| | | variable region | | SEQ ID NO: 743 | |
| MP267 | hNav1.7 | Heavy chain | H1 M834N | WO2014159595 | 19932 |
| | | variable region | | SEQ ID NO: 10 | |
| MP268 | hNav1.7 | Heavy chain | H1 M836N | WO2014159595 | 19933 |
| | | variable region | | SEQ ID NO: 759 | |
| MP269 | hNav1.7 | Heavy chain | H1 M839N | WO2014159595 | 19934 |
| | | variable region | | SEQ ID NO: 14 | |
| MP270 | hNav1.7 | Heavy chain | H1 M852N | WO2014159595 | 19935 |
| | | variable region | | SEQ ID NO: 18 | |
| MP271 | hNav1.7 | Heavy chain | H1 M875N | WO2014159595 | 19936 |
| | | variable region | | SEQ ID NO: 22 | |
| MP272 | hNav1.7 | Heavy chain | H1 M879N | WO2014159595 | 19937 |
| | | variable region | | SEQ ID NO: 791 | |
| MP273 | hNav1.7 | Heavy chain | H1 M994N | WO2014159595 | 19938 |
| | | variable region | | SEQ ID NO: 807 | |
| MP274 | hNav1.7 | Heavy chain | H1H1003B | WO2014159595 | 19939 |
| | | variable region | | SEQ ID NO: 98 | |
| MP275 | hNav1.7 | Heavy chain | H1H1006B | WO2014159595 | 19940 |
| | | variable region | | SEQ ID NO: 102 | |
| MP276 | hNav1.7 | Heavy chain | H1H1008B | WO2014159595 | 19941 |
| | | variable region | | SEQ ID NO: 106 | |
| MP277 | hNav1.7 | Heavy chain | H1H1010B | WO2014159595 | 19942 |
| | | variable region | | SEQ ID NO: 114 | |
| MP278 | hNav1.7 | Heavy chain | H1H1011B | WO2014159595 | 19943 |
| | | variable region | | SEQ ID NO: 118 | |
| MP279 | hNav1.7 | Heavy chain | H1H1013B | WO2014159595 | 19944 |
| | | variable region | | SEQ ID NO: 122 | |
| MP280 | TNF | Heavy chain | | US20030157061 | 19945 |
| | | variable region | | SEQ ID NO: 2 | |
| MP281 | TNF | Heavy chain | | US20030157061 | 19946 |
| | | variable region | | SEQ ID NO: 6 | |
| MP282 | TrkA | Heavy chain | HuVHWO | WO2009098238 | 19947 |
| | | variable region | | SEQ ID NO: 17 | |
| MP283 | NGF | Heavy chain, | Fulranumab, | U.S. Pat. No. 7,601,818 | 19948 |
| | | Antibody for | 4D4, AMG- | SEQ ID NO: 40 | |
| | | chronic pain | 403, JNJ- | | |
| | | | 42160443 | | |
| MP284 | NGF | Heavy chain, | Fasinumab, | | 19949 |
| | | Antibody for | REGN475, | | |
| | | chronic pain | SAR164877 | | |
| MP285 | NGF | Heavy chain, | Tanezumab, | US20040237124 | 19950 |
| | | Antibody for | PF- | SEQ ID NO: 1 | |
| | | pain, chronic | 04383119, | | |
| | | and acute, | RN624, E3 | | |
| | | osteoarthritis | | | |
| MP286 | CGRP | Light chain | G1, cluster | U.S. Pat. No. 9,115,194 | 19951 |
| | | | headache | SEQ ID NO: 12 | |
| MP287 | CGRP | Light chain | 04E4 | U.S. Pat. No. 9,102,731 | 19952 |
| | | | | SEQ ID NO: 17 | |
| MP288 | CGRP | Light chain | 10E4 | U.S. Pat. No. 9,102,731 | 19953 |
| | | | | SEQ ID NO: 22 | |
| MP289 | CGRP | Light chain | 02E7 | U.S. Pat. No. 9,102,731 | 19954 |
| | | | | SEQ ID NO: 14 | |
| MP290 | CGRP | Light chain | 12E8 | U.S. Pat. No. 9,102,731 | 19955 |
| | | | | SEQ ID NO: 25 | |
| MP291 | CGRP | Light chain | 01E11 | U.S. Pat. No. 9,102,731 | 19956 |
| | | | | SEQ ID NO: 12 | |
| MP292 | CGRP | Light chain | 01H7 | U.S. Pat. No. 9,102,731 | 19957 |
| | | | | SEQ ID NO: 13 | |
| MP293 | CGRP | Light chain | 03B6 | U.S. Pat. No. 9,102,731 | 19958 |
| | | | | SEQ ID NO: 15 | |
| MP294 | CGRP | Light chain | 03C8 | U.S. Pat. No. 9,102,731 | 19959 |
| | | | | SEQ ID NO: 16 | |
| MP295 | CGRP | Light chain | 04H6 | U.S. Pat. No. 9,102,731 | 19960 |
| | | | | SEQ ID NO: 18 | |
| MP296 | CGRP | Light chain | 05F5 | U.S. Pat. No. 9,102,731 | 19961 |
| | | | | SEQ ID NO: 19 | |
| MP297 | CGRP | Light chain | 09D4 | U.S. Pat. No. 9,102,731 | 19962 |
| | | | | SEQ ID NO: 20 | |
| MP298 | CGRP | Light chain | 09F5 | U.S. Pat. No. 9,102,731 | 19963 |
| | | | | SEQ ID NO: 21 | |
| MP299 | CGRP | Light chain | 11D11 HL | U.S. Pat. No. 9,102,731 | 19964 |
| | | | | SEQ ID NO: 23 | |
| MP300 | CGRP | Light chain | 11H9 | U.S. Pat. No. 9,102,731 | 19965 |
| | | | | SEQ ID NO: 24 | |
| MP301 | CGRP | Light chain | 12G8 HL | U.S. Pat. No. 9,102,731 | 19966 |
| | | | | SEQ ID NO: 26 | |
| MP302 | CGRP | Light chain | 13H2 | U.S. Pat. No. 9,102,731 | 19967 |
| | | | | SEQ ID NO: 27 | |
| MP303 | CGRP | Light chain | 32H7 | U.S. Pat. No. 9,102,731 | 19968 |
| | | | | SEQ ID NO: 28 | |
| MP304 | CGRP | Light chain | A | US20120294802 | 19969 |
| | | | | SEQ ID NO: 1 | |
| MP305 | CGRP | Light chain | Ab1 | US20120294802 | 19970 |
| | | | | SEQ ID NO: 2 | |
| MP306 | CGRP | Light chain | Ab10 | US20120294802 | 19971 |
| | | | | SEQ ID NO: 92 | |
| MP307 | CGRP | Light chain | Ab11 | US20120294802 | 19972 |
| | | | | SEQ ID NO: 102 | |
| MP308 | CGRP | Light chain | Ab12 | US20120294802 | 19973 |
| | | | | SEQ ID NO: 112 | |
| MP309 | CGRP | Light chain | Ab13 | US20120294802 | 19974 |
| | | | | SEQ ID NO: 122 | |
| MP310 | CGRP | Light chain | Ab14 | US20120294802 | 19975 |
| | | | | SEQ ID NO: 132 | |
| MP311 | CGRP | Light chain | Ab2 | US20120294802 | 19976 |
| | | | | SEQ ID NO: 12 | |
| MP312 | CGRP | Light chain | Ab3 | US20120294802 | 19977 |
| | | | | SEQ ID NO: 22 | |
| MP313 | CGRP | Light chain | Ab4 | US20120294802 | 19978 |
| | | | | SEQ ID NO: 32 | |
| MP314 | CGRP | Light chain | Ab5 | US20120294802 | 19979 |
| | | | | SEQ ID NO: 42 | |
| MP315 | CGRP | Light chain | Ab6 | US20120294802 | 19980 |
| | | | | SEQ ID NO: 52 | |
| MP316 | CGRP | Light chain | Ab7 | US20120294802 | 19981 |
| | | | | SEQ ID NO: 62 | |
| MP317 | CGRP | Light chain | Ab8 | US20120294802 | 19982 |
| | | | | SEQ ID NO: 72 | |
| MP318 | CGRP | Light chain | Ab9 | US20120294802 | 19983 |
| | | | | SEQ ID NO: 82 | |
| MP319 | CGRP | Light chain | B | US20120294802 | 19984 |
| | | | | SEQ ID NO: 11 | |
| MP320 | CGRP | Light chain | C | US20120294802 | 19985 |
| | | | | SEQ ID NO: 21 | |
| MP321 | CGRP | Light chain | D | US20120294802 | 19986 |
| | | | | SEQ ID NO: 31 | |
| MP322 | CGRP | Light chain | E | US20120294802 | 19987 |
| | | | | SEQ ID NO: 41 | |
| MP323 | CGRP | Light chain | F | US20120294802 | 19988 |
| | | | | SEQ ID NO: 51 | |
| MP324 | CGRP | Light chain | G | US20120294802 | 19989 |
| | | | | SEQ ID NO: 61 | |
| MP325 | CGRP | Light chain | H | US20120294802 | 19990 |
| | | | | SEQ ID NO: 71 | |
| MP326 | CGRP | Light chain | I | US20120294802 | 19991 |
| | | | | SEQ ID NO: 81 | |
| MP327 | CGRP | Light chain | J | US20120294802 | 19992 |
| | | | | SEQ ID NO: 91 | |
| MP328 | CGRP | Light chain | K | US20120294802 | 19993 |
| | | | | SEQ ID NO: 101 | |
| MP329 | CGRP | Light chain | L | US20120294802 | 19994 |
| | | | | SEQ ID NO: 111 | |
| MP330 | CGRP | Light chain | M | US20120294802 | 19995 |
| | | | | SEQ ID NO: 121 | |
| MP331 | CGRP | Light chain | N | US20120294802 | 19996 |
| | | | | SEQ ID NO: 131 | |
| MP332 | TrkA | Light chain | BXhVH5VL1, | US20150183885 | 19997 |
| | | | GBR | SEQ ID NO: 29 | |
| | | | VH5(K3Q)VL1, | | |
| | | | GBR | | |
| | | | VH5(V37A)VL1, | | |
| | | | GBR | | |
| | | | VH5(V37A)VL1(*), | | |
| | | | GBR | | |
| | | | VH5(G42E)VL1, | | |
| | | | GBR | | |
| | | | VH5(V89L)VL1, | | |
| | | | GBR | | |
| | | | VH5(R94K)VL1, | | |
| | | | GBR | | |
| | | | VH5(K3Q, | | |
| | | | V37A)VL1, | | |
| | | | GBR | | |
| | | | VH5(K3Q, | | |
| | | | V37A)VL1(*), | | |
| | | | GBR | | |
| | | | VH5(K3Q, | | |
| | | | T40A)VL1, | | |
| | | | GBR | | |
| | | | VH5(P60A, | | |
| | | | T62S)VL1, | | |
| | | | GBR | | |
| | | | VH5(K3Q, | | |
| | | | V37A, | | |
| | | | R44G)VL1, | | |
| | | | GBR | | |
| | | | VH5(K3Q, | | |
| | | | A49S, | | |
| | | | Y50A)VL1, | | |
| | | | GBR | | |
| | | | VH5(K3Q, | | |
| | | | P60A, | | |
| | | | T62S)VL1, | | |
| | | | GBR | | |
| | | | VH5(K3Q, | | |
| | | | T40A, | | |
| | | | P60A, T62S)VL1, | | |
| | | | GBR | | |
| | | | VH5(K3Q, | | |
| | | | V37A, | | |
| | | | T40A, P60A, | | |
| | | | T62S)VL1, | | |
| | | | GBR | | |
| | | | VH5(K3Q, | | |
| | | | T40A, | | |
| | | | R44G, A49S, | | |
| | | | Y50A)VL1, | | |
| | | | GBR | | |
| | | | VH5(K3Q, | | |
| | | | A49S, | | |
| | | | Y50A, P60A, | | |
| | | | T62S)VL1, | | |
| | | | GBR | | |
| | | | VH5(K3Q, | | |
| | | | T40A, R44G, | | |
| | | | A49S, Y50A, | | |
| | | | P60A, | | |
| | | | T62S)VL1, | | |
| | | | GBR | | |
| | | | VH5(K3Q, | | |
| | | | T40A, R44G, | | |
| | | | A49S, Y50A, | | |
| | | | P60A, T62S, | | |
| | | | R94K)VL1 | | |
| MP333 | TrkA | Light chain | BXhVL2 | WO2009098238 | 19998 |
| | | | | SEQ ID NO: 8 | |
| MP334 | TrkA | Light chain | BXhVL3 | WO2009098238 | 19999 |
| | | | | SEQ ID NO: 9 | |
| MP335 | TrkA | Light chain | BXhVL4 | WO2009098238 | 20000 |
| | | | | SEQ ID NO: 10 | |
| MP336 | TrkA | Light chain | BXhVL5 | WO2009098238 | 20001 |
| | | | | SEQ ID NO: 11 | |
| MP337 | TrkA | Light chain | BXhVL7 | WO2009098238 | 20002 |
| | | | | SEQ ID NO: 13 | |
| MP338 | TrkA | Light chain | BXhVL8 | WO2009098238 | 20003 |
| | | | | SEQ ID NO: 14 | |
| MP339 | TrkA | Light chain | BXhVL1 | WO2009098238 | 20004 |
| | | | | SEQ ID NO: 7 | |
| MP340 | TrkA | Light chain | BXhVLβ | WO2009098238 | 20005 |
| | | | | SEQ ID NO: 12 | |
| MP341 | TrkA | Light chain | mVLEP | WO2009098238 | 20006 |
| | | | | SEQ ID NO: 16 | |
| MP342 | GFRα3 | Light chain | H1M2236N | U.S. Pat. No. 8,968,736 | 20007 |
| | | variable region | | SEQ ID NO: 405 | |
| MP343 | GFRα3 | Light chain | H1M2243N | U.S. Pat. No. 8,968,736 | 20008 |
| | | variable region | | SEQ ID NO: 389 | |
| MP344 | GFRα3 | Light chain | H4H2207N | U.S. Pat. No. 8,968,736 | 20009 |
| | | variable region | | SEQ ID NO: 10 | |
| MP345 | GFRα3 | Light chain | H4H2210N | U.S. Pat. No. 8,968,736 | 20010 |
| | | variable region | | SEQ ID NO: 74 | |
| MP346 | GFRα3 | Light chain | H4H2212N | U.S. Pat. No. 8,968,736 | 20011 |
| | | variable region | | SEQ ID NO: 26 | |
| MP347 | GFRα3 | Light chain | H4H2234N | U.S. Pat. No. 8,968,736 | 20012 |
| | | variable region | | SEQ ID NO: 90 | |
| MP348 | GFRα3 | Light chain | H4H2236N3 | U.S. Pat. No. 8,968,736 | 20013 |
| | | variable region | | SEQ ID NO: 42 | |
| MP349 | GFRα3 | Light chain | H4H2243N2 | U.S. Pat. No. 8,968,736 | 20014 |
| | | variable region | | SEQ ID NO: 58 | |
| MP350 | GFRα3 | Light chain | H4H2291S | U.S. Pat. No. 8,968,736 | 20015 |
| | | variable region | | SEQ ID NO: 106 | |
| MP351 | GFRα3 | Light chain | H4H2292S | U.S. Pat. No. 8,968,736 | 20016 |
| | | variable region | | SEQ ID NO: 122 | |
| MP352 | GFRα3 | Light chain | H4H2293P | U.S. Pat. No. 8,968,736 | 20017 |
| | | variable region | | SEQ ID NO: 138 | |
| MP353 | GFRα3 | Light chain | H4H2294S | U.S. Pat. No. 8,968,736 | 20018 |
| | | variable region | | SEQ ID NO: 154 | |
| MP354 | GFRα3 | Light chain | H4H2295S | U.S. Pat. No. 8,968,736 | 20019 |
| | | variable region | | SEQ ID NO: 170 | |
| MP355 | GFRα3 | Light chain | H4H2296S | U.S. Pat. No. 8,968,736 | 20020 |
| | | variable region | | SEQ ID NO: 186 | |
| MP356 | GFRα3 | Light chain | H4H2341S | U.S. Pat. No. 8,968,736 | 20021 |
| | | variable region | | SEQ ID NO: 202 | |
| MP357 | GFRα3 | Light chain | H4H2342P | U.S. Pat. No. 8,968,736 | 20022 |
| | | variable region | | SEQ ID NO: 218 | |
| MP358 | GFRα3 | Light chain | H4H2344S | U.S. Pat. No. 8,968,736 | 20023 |
| | | variable region | | SEQ ID NO: 234 | |
| MP359 | GFRα3 | Light chain | H4H2345S | U.S. Pat. No. 8,968,736 | 20024 |
| | | variable region | | SEQ ID NO: 250 | |
| MP360 | GFRα3 | Light chain | H4H2346S | U.S. Pat. No. 8,968,736 | 20025 |
| | | variable region | | SEQ ID NO: 266 | |
| MP361 | GFRα3 | Light chain | H4H2350P | U.S. Pat. No. 8,968,736 | 20026 |
| | | variable region | | SEQ ID NO: 282 | |
| MP362 | GFRα3 | Light chain | H4H2352S | U.S. Pat. No. 8,968,736 | 20027 |
| | | variable region | | SEQ ID NO: 298 | |
| MP363 | GFRα3 | Light chain | H4H2354S | U.S. Pat. No. 8,968,736 | 20028 |
| | | variable region | | SEQ ID NO: 314 | |
| MP364 | GFRα3 | Light chain | H4H2355S | U.S. Pat. No. 8,968,736 | 20029 |
| | | variable region | | SEQ ID NO: 330 | |
| MP365 | GFRα3 | Light chain | H4H2357S | U.S. Pat. No. 8,968,736 | 20030 |
| | | variable region | | SEQ ID NO: 346 | |
| MP366 | GFRα3 | Light chain | H4H2364S | U.S. Pat. No. 8,968,736 | 20031 |
| | | variable region | | SEQ ID NO: 362 | |
| MP367 | hNav1.7 | Light chain | H1 H1 105B | WO2014159595 | 20032 |
| | | variable region | | SEQ ID NO: 200 | |
| MP368 | hNav1.7 | Light chain | H1 H1 138B | WO2014159595 | 20033 |
| | | variable region | | SEQ ID NO: 208 | |
| MP369 | hNav1.7 | Light chain | H1 H1 144B | WO2014159595 | 20034 |
| | | variable region | | SEQ ID NO: 212 | |
| MP370 | hNav1.7 | Light chain | H1 H1 147B | WO2014159595 | 20035 |
| | | variable region | | SEQ ID NO: 216 | |
| MP371 | hNav1.7 | Light chain | H1 H1 155B | WO2014159595 | 20036 |
| | | variable region | | SEQ ID NO: 220 | |
| MP372 | hNav1.7 | Light chain | H1 H1 164B | WO2014159595 | 20037 |
| | | variable region | | SEQ ID NO: 224 | |
| MP373 | hNav1.7 | Light chain | H1 H1 166B | WO2014159595 | 20038 |
| | | variable region | | SEQ ID NO: 228 | |
| MP374 | hNav1.7 | Light chain | H1 H1 169B | WO2014159595 | 20039 |
| | | variable region | | SEQ ID NO: 232 | |
| MP375 | hNav1.7 | Light chain | H1 H1006P | WO2014159595 | 20040 |
| | | variable region | | SEQ ID NO: 707 | |
| MP376 | hNav1.7 | Light chain | H1 H1025B | WO2014159595 | 20041 |
| | | variable region | | SEQ ID NO: 724 | |
| MP377 | hNav1.7 | Light chain | H1 H1068B | WO2014159595 | 20042 |
| | | variable region | | SEQ ID NO: 711 | |
| MP378 | hNav1.7 | Light chain | H1 H1069B | WO2014159595 | 20043 |
| | | variable region | | SEQ ID NO: 188 | |
| MP379 | hNav1.7 | Light chain | H1 H1082B | WO2014159595 | 20044 |
| | | variable region | | SEQ ID NO: 192 | |
| MP380 | hNav1.7 | Light chain | H1 H1098B | WO2014159595 | 20045 |
| | | variable region | | SEQ ID NO: 196 | |
| MP381 | hNav1.7 | Light chain | H1 M683N | WO2014159595 | 20046 |
| | | variable region | | SEQ ID NO: 4 | |
| MP382 | hNav1.7 | Light chain | H1 M797N | WO2014159595 | 20047 |
| | | variable region | | SEQ ID NO: 8 | |
| MP383 | hNav1.7 | Light chain | H1 M799N | WO2014159595 | 20048 |
| | | variable region | | SEQ ID NO: 28 | |
| MP384 | hNav1.7 | Light chain | H1 M801 N | WO2014159595 | 20049 |
| | | variable region | | SEQ ID NO: 735 | |
| MP385 | hNav1.7 | Light chain | H1 M826N | WO2014159595 | 20050 |
| | | variable region | | SEQ ID NO: 751 | |
| MP386 | hNav1.7 | Light chain | H1 M834N | WO2014159595 | 20051 |
| | | variable region | | SEQ ID NO: 12 | |
| MP387 | hNav1.7 | Light chain | H1 M836N | WO2014159595 | 20052 |
| | | variable region | | SEQ ID NO: 767 | |
| MP388 | hNav1.7 | Light chain | H1 M839N | WO2014159595 | 20053 |
| | | variable region | | SEQ ID NO: 16 | |
| MP389 | hNav1.7 | Light chain | H1 M852N | WO2014159595 | 20054 |
| | | variable region | | SEQ ID NO: 20 | |
| MP390 | hNav1.7 | Light chain | H1 M875N | WO2014159595 | 20055 |
| | | variable region | | SEQ ID NO: 24 | |
| MP391 | hNav1.7 | Light chain | H1 M879N | WO2014159595 | 20056 |
| | | variable region | | SEQ ID NO: 799 | |
| MP392 | hNav1.7 | Light chain | H1 M994N | WO2014159595 | 20057 |
| | | variable region | | SEQ ID NO: 815 | |
| MP393 | hNav1.7 | Light chain | H1H1002B | WO2014159595 | 20058 |
| | | variable region | | SEQ ID NO: 548 | |
| MP394 | hNav1.7 | Light chain | H1H1003B | WO2014159595 | 20059 |
| | | variable region | | SEQ ID NO: 100 | |
| MP395 | hNav1.7 | Light chain | H1H1005B | WO2014159595 | 20060 |
| | | variable region | | SEQ ID NO: 550 | |
| MP396 | hNav1.7 | Light chain | H1H1006B | WO2014159595 | 20061 |
| | | variable region | | SEQ ID NO: 104 | |
| MP397 | hNav1.7 | Light chain | H1H1008B | WO2014159595 | 20062 |
| | | variable region | | SEQ ID NO: 108 | |
| MP398 | hNav1.7 | Light chain | H1H1009B | WO2014159595 | 20063 |
| | | variable region | | SEQ ID NO: 552 | |
| MP399 | hNav1.7 | Light chain | H1H1010B | WO2014159595 | 20064 |
| | | variable region | | SEQ ID NO: 116 | |
| MP400 | hNav1.7 | Light chain | H1H1011B | WO2014159595 | 20065 |
| | | variable region | | SEQ ID NO: 120 | |
| MP401 | hNav1.7 | Light chain | H1H1013B | WO2014159595 | 20066 |
| | | variable region | | SEQ ID NO: 124 | |
| MP402 | hNav1.7 | Light chain | H1H1015B | WO2014159595 | 20067 |
| | | variable region | | SEQ ID NO: 128 | |
| MP403 | hNav1.7 | Light chain | H1H1016B | WO2014159595 | 20068 |
| | | variable region | | SEQ ID NO: 554 | |
| MP404 | hNav1.7 | Light chain | H1H1019B | WO2014159595 | 20069 |
| | | variable region | | SEQ ID NO: 112 | |
| MP405 | hNav1.7 | Light chain | H1H1020B | WO2014159595 | 20070 |
| | | variable region | | SEQ ID NO: 556 | |
| MP406 | hNav1.7 | Light chain | H1H1022B | WO2014159595 | 20071 |
| | | variable region | | SEQ ID NO: 132 | |
| MP407 | hNav1.7 | Light chain | H1H1023B | WO2014159595 | 20072 |
| | | variable region | | SEQ ID NO: 136 | |
| MP408 | hNav1.7 | Light chain | H1H1024B | WO2014159595 | 20073 |
| | | variable region | | SEQ ID NO: 558 | |
| MP409 | hNav1.7 | Light chain | H1H1025B | WO2014159595 | 20074 |
| | | variable region | | SEQ ID NO: 560 | |
| MP410 | hNav1.7 | Light chain | H1H1026B | WO2014159595 | 20075 |
| | | variable region | | SEQ ID NO: 140 | |
| MP411 | hNav1.7 | Light chain | H1H1030B | WO2014159595 | 20076 |
| | | variable region | | SEQ ID NO: 144 | |
| MP412 | hNav1.7 | Light chain | H1H1032B | WO2014159595 | 20077 |
| | | variable region | | SEQ ID NO: 148 | |
| MP413 | hNav1.7 | Light chain | H1H1034B | WO2014159595 | 20078 |
| | | variable region | | SEQ ID NO: 562 | |
| MP414 | hNav1.7 | Light chain | H1H1035B | WO2014159595 | 20079 |
| | | variable region | | SEQ ID NO: 564 | |
| MP415 | hNav1.7 | Light chain | H1H1038B | WO2014159595 | 20080 |
| | | variable region | | SEQ ID NO: 152 | |
| MP416 | hNav1.7 | Light chain | H1H1041B | WO2014159595 | 20081 |
| | | variable region | | SEQ ID NO: 156 | |
| MP417 | hNav1.7 | Light chain | H1H1044B | WO2014159595 | 20082 |
| | | variable region | | SEQ ID NO: 160 | |
| MP418 | hNav1.7 | Light chain | H1H1045B | WO2014159595 | 20083 |
| | | variable region | | SEQ ID NO: 164 | |
| MP419 | hNav1.7 | Light chain | H1H1048B | WO2014159595 | 20084 |
| | | variable region | | SEQ ID NO: 566 | |
| MP420 | hNav1.7 | Light chain | H1H1049B | WO2014159595 | 20085 |
| | | variable region | | SEQ ID NO: 568 | |
| MP421 | hNav1.7 | Light chain | H1H1050B | WO2014159595 | 20086 |
| | | variable region | | SEQ ID NO: 168 | |
| MP422 | hNav1.7 | Light chain | H1H1051B | WO2014159595 | 20087 |
| | | variable region | | SEQ ID NO: 570 | |
| MP423 | hNav1.7 | Light chain | H1H1055B | WO2014159595 | 20088 |
| | | variable region | | SEQ ID NO: 172 | |
| MP424 | hNav1.7 | Light chain | H1H1056B | WO2014159595 | 20089 |
| | | variable region | | SEQ ID NO: 176 | |
| MP425 | hNav1.7 | Light chain | H1H1059B | WO2014159595 | 20090 |
| | | variable region | | SEQ ID NO: 180 | |
| MP426 | hNav1.7 | Light chain | H1H1060B | WO2014159595 | 20091 |
| | | variable region | | SEQ ID NO: 184 | |
| MP427 | hNav1.7 | Light chain | H1H1064B | WO2014159595 | 20092 |
| | | variable region | | SEQ ID NO: 572 | |
| MP428 | hNav1.7 | Light chain | H1H1071B | WO2014159595 | 20093 |
| | | variable region | | SEQ ID NO: 574 | |
| MP429 | hNav1.7 | Light chain | H1H1072B | WO2014159595 | 20094 |
| | | variable region | | SEQ ID NO: 576 | |
| MP430 | hNav1.7 | Light chain | H1H1077B | WO2014159595 | 20095 |
| | | variable region | | SEQ ID NO: 578 | |
| MP431 | hNav1.7 | Light chain | H1H1086B | WO2014159595 | 20096 |
| | | variable region | | SEQ ID NO: 580 | |
| MP432 | hNav1.7 | Light chain | H1H1096B | WO2014159595 | 20097 |
| | | variable region | | SEQ ID NO: 582 | |
| MP433 | hNav1.7 | Light chain | H1H1120B | WO2014159595 | 20098 |
| | | variable region | | SEQ ID NO: 584 | |
| MP434 | hNav1.7 | Light chain | H1H1128B | WO2014159595 | 20099 |
| | | variable region | | SEQ ID NO: 586 | |
| MP435 | hNav1.7 | Light chain | H1H1132B | WO2014159595 | 20100 |
| | | variable region | | SEQ ID NO: 588 | |
| MP436 | hNav1.7 | Light chain | H1H1142B | WO2014159595 | 20101 |
| | | variable region | | SEQ ID NO: 590 | |
| MP437 | hNav1.7 | Light chain | H1H1171B | WO2014159595 | 20102 |
| | | variable region | | SEQ ID NO: 592 | |
| MP438 | hNav1.7 | Light chain | H2M799N | WO2014159595 | 20103 |
| | | variable region | | SEQ ID NO: 831 | |
| MP439 | hNav1.7 | Light chain | H4H1003P | WO2014159595 | 20104 |
| | | variable region | | SEQ ID NO: 862 | |
| MP440 | hNav1.7 | Light chain | H4H1025P | WO2014159595 | 20105 |
| | | variable region | | SEQ ID NO: 874 | |
| MP441 | hNav1.7 | Light chain | H4H361 B | WO2014159595 | 20106 |
| | | variable region | | SEQ ID NO: 236 | |
| MP442 | hNav1.7 | Light chain | H4H362B | WO2014159595 | 20107 |
| | | variable region | | SEQ ID NO: 32 | |
| MP443 | hNav1.7 | Light chain | H4H362P | WO2014159595 | 20108 |
| | | variable region | | SEQ ID NO: 870 | |
| MP444 | hNav1.7 | Light chain | H4H363B | WO2014159595 | 20109 |
| | | variable region | | SEQ ID NO: 594 | |
| MP445 | hNav1.7 | Light chain | H4H364B | WO2014159595 | 20110 |
| | | variable region | | SEQ ID NO: 596 | |
| MP446 | hNav1.7 | Light chain | H4H365B | WO2014159595 | 20111 |
| | | variable region | | SEQ ID NO: 240 | |
| MP447 | hNav1.7 | Light chain | H4H366B | WO2014159595 | 20112 |
| | | variable region | | SEQ ID NO: 598 | |
| MP448 | hNav1.7 | Light chain | H4H367B | WO2014159595 | 20113 |
| | | variable region | | SEQ ID NO: 36 | |
| MP449 | hNav1.7 | Light chain | H4H368B | WO2014159595 | 20114 |
| | | variable region | | SEQ ID NO: 40 | |
| MP450 | hNav1.7 | Light chain | H4H369B | WO2014159595 | 20115 |
| | | variable region | | SEQ ID NO: 600 | |
| MP451 | hNav1.7 | Light chain | H4H371 B | WO2014159595 | 20116 |
| | | variable region | | SEQ ID NO: 244 | |
| MP452 | hNav1.7 | Light chain | H4H372B | WO2014159595 | 20117 |
| | | variable region | | SEQ ID NO: 248 | |
| MP453 | hNav1.7 | Light chain | H4H373B | WO2014159595 | 20118 |
| | | variable region | | SEQ ID NO: 252 | |
| MP454 | hNav1.7 | Light chain | H4H374B | WO2014159595 | 20119 |
| | | variable region | | SEQ ID NO: 602 | |
| MP455 | hNav1.7 | Light chain | H4H375B | WO2014159595 | 20120 |
| | | variable region | | SEQ ID NO: 604 | |
| MP456 | hNav1.7 | Light chain | H4H376B | WO2014159595 | 20121 |
| | | variable region | | SEQ ID NO: 606 | |
| MP457 | hNav1.7 | Light chain | H4H377B | WO2014159595 | 20122 |
| | | variable region | | SEQ ID NO: 608 | |
| MP458 | hNav1.7 | Light chain | H4H379B | WO2014159595 | 20123 |
| | | variable region | | SEQ ID NO: 256 | |
| MP459 | hNav1.7 | Light chain | H4H380B | WO2014159595 | 20124 |
| | | variable region | | SEQ ID NO: 610 | |
| MP460 | hNav1.7 | Light chain | H4H381 B | WO2014159595 | 20125 |
| | | variable region | | SEQ ID NO: 260 | |
| MP461 | hNav1.7 | Light chain | H4H382B | WO2014159595 | 20126 |
| | | variable region | | SEQ ID NO: 44 | |
| MP462 | hNav1.7 | Light chain | H4H384B | WO2014159595 | 20127 |
| | | variable region | | SEQ ID NO: 612 | |
| MP463 | hNav1.7 | Light chain | H4H385B | WO2014159595 | 20128 |
| | | variable region | | SEQ ID NO: 264 | |
| MP464 | hNav1.7 | Light chain | H4H385B | WO2014159595 | 20129 |
| | | variable region | | SEQ ID NO: 683 | |
| MP465 | hNav1.7 | Light chain | H4H387B | WO2014159595 | 20130 |
| | | variable region | | SEQ ID NO: 614 | |
| MP466 | hNav1.7 | Light chain | H4H388B | WO2014159595 | 20131 |
| | | variable region | | SEQ ID NO: 268 | |
| MP467 | hNav1.7 | Light chain | H4H391B | WO2014159595 | 20132 |
| | | variable region | | SEQ ID NO: 48 | |
| MP468 | hNav1.7 | Light chain | H4H391P | WO2014159595 | 20133 |
| | | variable region | | SEQ ID NO: 52 | |
| MP469 | hNav1.7 | Light chain | H4H392B | WO2014159595 | 20134 |
| | | variable region | | SEQ ID NO: 616 | |
| MP470 | hNav1.7 | Light chain | H4H394B | WO2014159595 | 20135 |
| | | variable region | | SEQ ID NO: 618 | |
| MP471 | hNav1.7 | Light chain | H4H395B | WO2014159595 | 20136 |
| | | variable region | | SEQ ID NO: 620 | |
| MP472 | hNav1.7 | Light chain | H4H395B | WO2014159595 | 20137 |
| | | variable region | | SEQ ID NO: 687 | |
| MP473 | hNav1.7 | Light chain | H4H396B | WO2014159595 | 20138 |
| | | variable region | | SEQ ID NO: 272 | |
| MP474 | hNav1.7 | Light chain | H4H397B | WO2014159595 | 20139 |
| | | variable region | | SEQ ID NO: 56 | |
| MP475 | hNav1.7 | Light chain | H4H398B | WO2014159595 | 20140 |
| | | variable region | | SEQ ID NO: 276 | |
| MP476 | hNav1.7 | Light chain | H4H399B | WO2014159595 | 20141 |
| | | variable region | | SEQ ID NO: 280 | |
| MP477 | hNav1.7 | Light chain | H4H400B | WO2014159595 | 20142 |
| | | variable region | | SEQ ID NO: 284 | |
| MP478 | hNav1.7 | Light chain | H4H402B | WO2014159595 | 20143 |
| | | variable region | | SEQ ID NO: 288 | |
| MP479 | hNav1.7 | Light chain | H4H404B | WO2014159595 | 20144 |
| | | variable region | | SEQ ID NO: 622 | |
| MP480 | hNav1.7 | Light chain | H4H408B | WO2014159595 | 20145 |
| | | variable region | | SEQ ID NO: 60 | |
| MP481 | hNav1.7 | Light chain | H4H409B | WO2014159595 | 20146 |
| | | variable region | | SEQ ID NO: 292 | |
| MP482 | hNav1.7 | Light chain | H4H411B | WO2014159595 | 20147 |
| | | variable region | | SEQ ID NO: 626 | |
| MP483 | hNav1.7 | Light chain | H4H410B | WO2014159595 | 20148 |
| | | variable region | | SEQ ID NO: 624 | |
| MP484 | hNav1.7 | Light chain | H4H412B | WO2014159595 | 20149 |
| | | variable region | | SEQ ID NO: 628 | |
| MP485 | hNav1.7 | Light chain | H4H414B | WO2014159595 | 20150 |
| | | variable region | | SEQ ID NO: 630 | |
| MP486 | hNav1.7 | Light chain | H4H415B | WO2014159595 | 20151 |
| | | variable region | | SEQ ID NO: 296 | |
| MP487 | hNav1.7 | Light chain | H4H416B | WO2014159595 | 20152 |
| | | variable region | | SEQ ID NO: 300 | |
| MP488 | hNav1.7 | Light chain | H4H419B | WO2014159595 | 20153 |
| | | variable region | | SEQ ID NO: 304 | |
| MP489 | hNav1.7 | Light chain | H4H421B | WO2014159595 | 20154 |
| | | variable region | | SEQ ID NO: 632 | |
| MP490 | hNav1.7 | Light chain | H4H422B | WO2014159595 | 20155 |
| | | variable region | | SEQ ID NO: 308 | |
| MP491 | hNav1.7 | Light chain | H4H426B | WO2014159595 | 20156 |
| | | variable region | | SEQ ID NO: 64 | |
| MP492 | hNav1.7 | Light chain | H4H428B | WO2014159595 | 20157 |
| | | variable region | | SEQ ID NO: 634 | |
| MP493 | hNav1.7 | Light chain | H4H430B | WO2014159595 | 20158 |
| | | variable region | | SEQ ID NO: 636 | |
| MP494 | hNav1.7 | Light chain | H4H431B | WO2014159595 | 20159 |
| | | variable region | | SEQ ID NO: 638 | |
| MP495 | hNav1.7 | Light chain | H4H433B | WO2014159595 | 20160 |
| | | variable region | | SEQ ID NO: 640 | |
| MP496 | hNav1.7 | Light chain | H4H434B | WO2014159595 | 20161 |
| | | variable region | | SEQ ID NO: 312 | |
| MP497 | hNav1.7 | Light chain | H4H434B | WO2014159595 | 20162 |
| | | variable region | | SEQ ID NO: 691 | |
| MP498 | hNav1.7 | Light chain | H4H434P | WO2014159595 | 20163 |
| | | variable region | | SEQ ID NO: 695 | |
| MP499 | hNav1.7 | Light chain | H4H435B | WO2014159595 | 20164 |
| | | variable region | | SEQ ID NO: 642 | |
| MP500 | hNav1.7 | Light chain | H4H438B | WO2014159595 | 20165 |
| | | variable region | | SEQ ID NO: 316 | |
| MP501 | hNav1.7 | Light chain | H4H438B | WO2014159595 | 20166 |
| | | variable region | | SEQ ID NO: 699 | |
| MP502 | hNav1.7 | Light chain | H4H439B | WO2014159595 | 20167 |
| | | variable region | | SEQ ID NO: 68 | |
| MP503 | hNav1.7 | Light chain | H4H439P | WO2014159595 | 20168 |
| | | variable region | | SEQ ID NO: 72 | |
| MP504 | hNav1.7 | Light chain | H4H440B | WO2014159595 | 20169 |
| | | variable region | | SEQ ID NO: 644 | |
| MP505 | hNav1.7 | Light chain | H4H441B | WO2014159595 | 20170 |
| | | variable region | | SEQ ID NO: 646 | |
| MP506 | hNav1.7 | Light chain | H4H441 B | WO2014159595 | 20171 |
| | | variable region | | SEQ ID NO: 703 | |
| MP507 | hNav1.7 | Light chain | H4H441 P | WO2014159595 | 20172 |
| | | variable region | | SEQ ID NO: 866 | |
| MP508 | hNav1.7 | Light chain | H4H442B | WO2014159595 | 20173 |
| | | variable region | | SEQ ID NO: 320 | |
| MP509 | hNav1.7 | Light chain | H4H443B | WO2014159595 | 20174 |
| | | variable region | | SEQ ID NO: 76 | |
| MP510 | hNav1.7 | Light chain | H4H444B | WO2014159595 | 20175 |
| | | variable region | | SEQ ID NO: 324 | |
| MP511 | hNav1.7 | Light chain | H4H446B | WO2014159595 | 20176 |
| | | variable region | | SEQ ID NO: 328 | |
| MP512 | hNav1.7 | Light chain | H4H448B | WO2014159595 | 20177 |
| | | variable region | | SEQ ID NO: 80 | |
| MP513 | hNav1.7 | Light chain | H4H450B | WO2014159595 | 20178 |
| | | variable region | | SEQ ID NO: 648 | |
| MP514 | hNav1.7 | Light chain | H4H451B | WO2014159595 | 20179 |
| | | variable region | | SEQ ID NO: 650 | |
| MP515 | hNav1.7 | Light chain | H4H452B | WO2014159595 | 20180 |
| | | variable region | | SEQ ID NO: 652 | |
| MP516 | hNav1.7 | Light chain | H4H455B | WO2014159595 | 20181 |
| | | variable region | | SEQ ID NO: 654 | |
| MP517 | hNav1.7 | Light chain | H4H456B | WO2014159595 | 20182 |
| | | variable region | | SEQ ID NO: 332 | |
| MP518 | hNav1.7 | Light chain | H4H457B | WO2014159595 | 20183 |
| | | variable region | | SEQ ID NO: 336 | |
| MP519 | hNav1.7 | Light chain | H4H458B | WO2014159595 | 20184 |
| | | variable region | | SEQ ID NO: 340 | |
| MP520 | hNav1.7 | Light chain | H4H459B | WO2014159595 | 20185 |
| | | variable region | | SEQ ID NO: 656 | |
| MP521 | hNav1.7 | Light chain | H4H460B | WO2014159595 | 20186 |
| | | variable region | | SEQ ID NO: 344 | |
| MP522 | hNav1.7 | Light chain | H4H461 B | WO2014159595 | 20187 |
| | | variable region | | SEQ ID NO: 348 | |
| MP523 | hNav1.7 | Light chain | H4H462B | WO2014159595 | 20188 |
| | | variable region | | SEQ ID NO: 352 | |
| MP524 | hNav1.7 | Light chain | H4H463B | WO2014159595 | 20189 |
| | | variable region | | SEQ ID NO: 356 | |
| MP525 | hNav1.7 | Light chain | H4H464B | WO2014159595 | 20190 |
| | | variable region | | SEQ ID NO: 360 | |
| MP526 | hNav1.7 | Light chain | H4H465B | WO2014159595 | 20191 |
| | | variable region | | SEQ ID NO: 364 | |
| MP527 | hNav1.7 | Light chain | H4H466B | WO2014159595 | 20192 |
| | | variable region | | SEQ ID NO: 368 | |
| MP528 | hNav1.7 | Light chain | H4H467B | WO2014159595 | 20193 |
| | | variable region | | SEQ ID NO: 372 | |
| MP529 | hNav1.7 | Light chain | H4H468B | WO2014159595 | 20194 |
| | | variable region | | SEQ ID NO: 84 | |
| MP530 | hNav1.7 | Light chain | H4H468P | WO2014159595 | 20195 |
| | | variable region | | SEQ ID NO: 88 | |
| MP531 | hNav1.7 | Light chain | H4H469B | WO2014159595 | 20196 |
| | | variable region | | SEQ ID NO: 658 | |
| MP532 | hNav1.7 | Light chain | H4H470B | WO2014159595 | 20197 |
| | | variable region | | SEQ ID NO: 660 | |
| MP533 | hNav1.7 | Light chain | H4H471B | WO2014159595 | 20198 |
| | | variable region | | SEQ ID NO: 92 | |
| MP534 | hNav1.7 | Light chain | H4H471P | WO2014159595 | 20199 |
| | | variable region | | SEQ ID NO: 96 | |
| MP535 | hNav1.7 | Light chain | H4H472B | WO2014159595 | 20200 |
| | | variable region | | SEQ ID NO: 376 | |
| MP536 | hNav1.7 | Light chain | H4H473B | WO2014159595 | 20201 |
| | | variable region | | SEQ ID NO: 380 | |
| MP537 | hNav1.7 | Light chain | H4H474B | WO2014159595 | 20202 |
| | | variable region | | SEQ ID NO: 662 | |
| MP538 | hNav1.7 | Light chain | H4H475B | WO2014159595 | 20203 |
| | | variable region | | SEQ ID NO: 384 | |
| MP539 | hNav1.7 | Light chain | H4H476B | WO2014159595 | 20204 |
| | | variable region | | SEQ ID NO: 664 | |
| MP540 | hNav1.7 | Light chain | H4H477B | WO2014159595 | 20205 |
| | | variable region | | SEQ ID NO: 388 | |
| MP541 | hNav1.7 | Light chain | H4H479B | WO2014159595 | 20206 |
| | | variable region | | SEQ ID NO: 666 | |
| MP542 | hNav1.7 | Light chain | H4H480B | WO2014159595 | 20207 |
| | | variable region | | SEQ ID NO: 392 | |
| MP543 | hNav1.7 | Light chain | H4H481 B | WO2014159595 | 20208 |
| | | variable region | | SEQ ID NO: 396 | |
| MP544 | hNav1.7 | Light chain | H4H482B | WO2014159595 | 20209 |
| | | variable region | | SEQ ID NO: 400 | |
| MP545 | hNav1.7 | Light chain | H4H483B | WO2014159595 | 20210 |
| | | variable region | | SEQ ID NO: 404 | |
| MP546 | hNav1.7 | Light chain | H4H484B | WO2014159595 | 20211 |
| | | variable region | | SEQ ID NO: 408 | |
| MP547 | hNav1.7 | Light chain | H4H486B | WO2014159595 | 20212 |
| | | variable region | | SEQ ID NO: 412 | |
| MP548 | hNav1.7 | Light chain | H4H487B | WO2014159595 | 20213 |
| | | variable region | | SEQ ID NO: 668 | |
| MP549 | hNav1.7 | Light chain | H4H488B | WO2014159595 | 20214 |
| | | variable region | | SEQ ID NO: 416 | |
| MP550 | hNav1.7 | Light chain | H4H489B | WO2014159595 | 20215 |
| | | variable region | | SEQ ID NO: 420 | |
| MP551 | hNav1.7 | Light chain | H4H491B | WO2014159595 | 20216 |
| | | variable region | | SEQ ID NO: 424 | |
| MP552 | TNF | Light chain | | US20030157061 | 20217 |
| | | variable region | | SEQ ID NO: 4 | |
| MP553 | TrkA | Light chain | 3-23*01 | WO2009098238 | 20218 |
| | | variable region | | SEQ ID NO: 19 | |
| MP554 | TrkA | Light chain | BXhVH5VL1 | WO2009098238 | 20219 |
| | | variable region | N297A i | SEQ ID NO: 23 | |
| MP555 | TrkA | Light chain | HuVLWO | WQ2009098238 | 20220 |
| | | variable region | | SEQ ID NO: 18 | |
| MP556 | TrkA | Light chain | JH4 | WO2009098238 | 20221 |
| | | variable region | | SEQ ID NO: 20 | |
| MP557 | TrkA | Light chain | JK1 | WO2009098238 | 20222 |
| | | variable region | | SEQ ID NO: 22 | |
| MP558 | TrkA | Light chain | L6*01 | WO2009098238 | 20223 |
| | | variable region | | SEQ ID NO: 21 | |
| MP559 | NGF | Light chain | Fasinumab, | U.S. Pat. No. 7,988,967 | 20224 |
| | | variable region, | REGN475, | SEQ ID NO: 110; | |
| | | Antibody for | SAR164877 | U.S. Pat. No. 7,988,967 | |
| | | chronic pain | | SEQ ID NO: 92 | |
| MP560 | NGF | Light chain, | Fulranumab, | U.S. Pat. No. 7,601,818 | 20225 |
| | | Antibody for | 4D4, AMG- | SEQ ID NO: 44; | |
| | | chronic pain | 403, JNJ- | U.S. Pat. No. 8,552,157 | |
| | | | 42160443 | SEQ ID NO: 17; | |
| | | | | U.S. Pat. No. 8,048,421 | |
| | | | | SEQ ID NO: 84 | |
| MP561 | NGF | Light chain, | Fasinumab, | | 20226 |
| | | Antibody for | REGN475, | | |
| | | chronic pain | SAR164877 | | |
| MP562 | NGF | Light chain, | Tanezumab, | US20040237124 | 20227 |
| | | Antibody for | PF-04383119, | SEQ ID NO: 2 | |
| | | pain, chronic | RN624, E3 | | |
| | | and acute, | | | |
| | | osteoarthritis | | | |
| MP563 | CGRP | Variable Heavy | G1, cluster | U.S. Pat. No. 9,115,194 | 20228 |
| | | Domain | headache | SEQ ID NO: 1 | |
| MP564 | CGRP | Variable Light | G1, cluster | U.S. Pat. No. 9,115,194 | 20229 |
| | | Domain | headache | SEQ ID NO: 2 | |

### Ocular Disease Antibodies

In some embodiments, the payload region of the viral particle comprises one or more nucleic acid sequences encoding one or more of the ocular disease payload antibody polypeptides listed in Table 11 (0C1-00676; SEQ ID NO: 20230-20905).

TABLE 11
Ocular Disease Antibodies

| Antibody No. | Target | Description | Antibody Name | Reference Information | SEQ ID NO |
|---|---|---|---|---|---|
| OC1 | VEGF-A | Fab-12 1cz8_L, | Ranibizumab, | Lien and Lowman, In: | 20230 |
| | | Fab-12 variant | Lucentis | Chemajovsky, 2008, Therapeutic | |
| | | Y0317/L- | | Antibodies. Handbook of | |
| | | KAPPA (V- | | Experimental Pharmacology 181, | |
| | | KAPPA(1- | | Springer-Verlag, Berlin | |
| | | 107) + C- | | Heidelberg 131-150 | |
| | | KAPPA(108- | | | |
| | | 213) | | | |
| OC2 | VEGF-A | Fab-12 variant | Ranibizumab, | Lien and Lowman, In: | 20231 |
| | | Y031/Fab-12 | Lucentis | Chemajovsky, 2008, Therapeutic | |
| | | variant Y0317 | | Antibodies. Handbook of | |
| | | and VH-CH1 | | Experimental Pharmacology 181, | |
| | | (VH(1- | | Springer-Verlag, Berlin | |
| | | 123) + CH1(124- | | Heidelberg 131-150 | |
| | | 215) | | | |
| OC3 | VEGF-A | Fab-12 variant | Ranibizumab, | Lien and Lowman, In: | 20232 |
| | | Y0317/L- | Lucentis | Chemajovsky, 2008, Therapeutic | |
| | | K APPA (V- | | Antibodies. Handbook of | |
| | | KAPPA(1- | | Experimental Pharmacology 181, | |
| | | 107) + C- | | Springer-Verlag, Berlin | |
| | | KAPPA(108- | | Heidelberg 131-150 | |
| | | 213) | | | |
| OC4 | VEGF-A | Fab-12 variant | Ranibizumab, | Lien and Lowman, In: | 20233 |
| | | Y0317/VH- | Lucentis | Chemajovsky, 2008, Therapeutic | |
| | | CH1 (VH(1- | | Antibodies. Handbook of | |
| | | 123) + CH1(124- | | Experimental Pharmacology 181, | |
| | | 215) | | Springer-Verlag, Berlin | |
| | | | | Heidelberg 131-150 | |
| OC5 | | Fusion protein | Aflibercept | | 20234 |
| | | | fusion protein | | |
| OC6 | | Fusion protein | Conbercept | | 20235 |
| | | | fusion protein | | |
| OC7 | Annexin IV or a | Heavy chain | B4 | WO2014116880 | 20236 |
| | phospholipid; | | | SEQ ID NO: 15 | |
| | and (b) a | | | | |
| | complement | | | | |
| | inhibitor | | | | |
| OC8 | Annexin IV or a | Heavy chain | B4 | WO2014116880 | 20237 |
| | phospholipid; | | | SEQ ID NO: 16 | |
| | and (b) a | | | | |
| | complement | | | | |
| | inhibitor | | | | |
| OC9 | Annexin IV or a | Heavy chain | C2 | WO2014116880 | 20238 |
| | phospholipid; | | | SEQ ID NO: 36 | |
| | and (b) a | | | | |
| | complement | | | | |
| | inhibitor | | | | |
| OC10 | C3b | Heavy chain | rhuMAB 4D5- | U.S. Pat. No. 8,377,437 | 20239 |
| | | | 8 | SEQ ID NO: 14 | |
| OC11 | C3b, Properdin | Heavy chain | H-6 | WO2015099838 | 20240 |
| | (factor P), | | | SEQ ID NO: 49 | |
| | Factors Ba and | | | | |
| | Bb, C5, C6, C7, | | | | |
| | C8, C9 | | | | |
| OC12 | C3b, Properdin | Heavy chain | H-7 | WO2015099838 | 20241 |
| | (factor P), | | | SEQ ID NO: 50 | |
| | Factors Ba and | | | | |
| | Bb, C5, C6, C7, | | | | |
| | C8, C9 | | | | |
| OC13 | C3b, Properdin | Heavy chain | H-8 | WO2015099838 | 20242 |
| | (factor P), | | | SEQ ID NO: 51 | |
| | Factors Ba and | | | | |
| | Bb, C5, C6, C7, | | | | |
| | C8. C9 | | | | |
| OC14 | C3b, Properdin | Heavy chain | H-9 | WO2015099838 | 20243 |
| | (factor P), | | | SEQ ID NO: 52 | |
| | Factors Ba and | | | | |
| | Bb, C5, C6, C7, | | | | |
| | C8, C9 | | | | |
| OC15 | C3b, Properdin | Heavy chain | H-10 | WO2015099838 | 20244 |
| | (factor P), | | | SEQ ID NO: 53 | |
| | Factors Ba and | | | | |
| | Bb, C5, C6, C7, | | | | |
| | C8, C9 | | | | |
| OC16 | C3b, Properdin | Heavy chain | H-11 | WO2015099838 | 20245 |
| | (factor P), | | | SEQ ID NO: 54 | |
| | Factors Ba and | | | | |
| | Bb, C5, C6, C7, | | | | |
| | C8, C9 | | | | |
| OC17 | C3b, Properdin | Heavy chain | H-12 | WO2015099838 | 20246 |
| | (factor P), | | | SEQ ID NO: 55 | |
| | Factors Ba and | | | | |
| | Bb, C5, C6, C7, | | | | |
| | C8, C9 | | | | |
| OC18 | C3b, Properdin | Heavy chain | H-13 | WO2015099838 | 20247 |
| | (factor P), | | | SEQ ID NO: 56 | |
| | Factors Ba and | | | | |
| | Bb, C5, C6, C7, | | | | |
| | C8, C9 | | | | |
| OC19 | C3b, Properdin | Heavy chain | H-14 | WO2015099838 | 20248 |
| | (factor P), | | | SEQ ID NO: 57 | |
| | Factors Ba and | | | | |
| | Bb, C5, C6, C7, | | | | |
| | C8, C9 | | | | |
| OC20 | C3b, Properdin | Heavy chain | H-15 | WO2015099838 | 20249 |
| | (factor P), | | | SEQ ID NO: 58 | |
| | Factors Ba and | | | | |
| | Bb, C5, C6, C7, | | | | |
| | C8, C9 | | | | |
| OC21 | C3b, Properdin | Heavy chain | H-16 | WO2015099838 | 20250 |
| | (factor P), | | | SEQ ID NO: 59 | |
| | Factors Ba and | | | | |
| | Bb, C5, C6, C7, | | | | |
| | C8, C9 | | | | |
| OC22 | C3b, Properdin | Heavy chain | H-17 | WO2015099838 | 20251 |
| | (factor P), | | | SEQ ID NO: 60 | |
| | Factors Ba and | | | | |
| | Bb, C5, C6, C7, | | | | |
| | C8, C9 | | | | |
| OC23 | C3b, Properdin | Heavy chain | H-18 | WO2015099838 | 20252 |
| | (factor P), | | | SEQ ID NO: 61 | |
| | Factors Ba and | | | | |
| | Bb, C5, C6, C7, | | | | |
| | C8, C9 | | | | |
| OC24 | C3b, Properdin | Heavy chain | H-19 | WO2015099838 | 20253 |
| | (factor P), | | | SEQ ID NO: 62 | |
| | Factors Ba and | | | | |
| | Bb, C5, C6, C7, | | | | |
| | C8, C9 | | | | |
| OC25 | C3b, Properdin | Heavy chain | H-20 | WO2015099838 | 20254 |
| | (factor P), | | | SEQ ID NO: 63 | |
| | Factors Ba and | | | | |
| | Bb, C5, C6, C7, | | | | |
| | C8, C9 | | | | |
| OC26 | C3b, Properdin | Heavy chain | H-21 | WO2015099838 | 20255 |
| | (factor P), | | | SEQ ID NO: 64 | |
| | Factors Ba and | | | | |
| | Bb, C5, C6, C7, | | | | |
| | C8, C9 | | | | |
| OC27 | C3b, Properdin | Heavy chain | H-22 | WO2015099838 | 20256 |
| | (factor P), | | | SEQ ID NO: 65 | |
| | Factors Ba and | | | | |
| | Bb, C5, C6, C7, | | | | |
| | C8, C9 | | | | |
| OC28 | C3b, Properdin | Heavy chain | H-23 | WO2015099838 | 20257 |
| | (factor P), | | | SEQ ID NO: 66 | |
| | Factors Ba and | | | | |
| | Bb, C5, C6, C7, | | | | |
| | C8, C9 | | | | |
| OC29 | C3b, Properdin | Heavy chain | H-24 | WO2015099838 | 20258 |
| | (factor P), | | | SEQ ID NO: 67 | |
| | Factors Ba and | | | | |
| | Bb, C5, C6, C7, | | | | |
| | C8, C9 | | | | |
| OC30 | C3b, Properdin | Heavy chain | H-25 | WO2015099838 | 20259 |
| | (factor P), | | | SEQ ID NO: 68 | |
| | Factors Ba and | | | | |
| | Bb, C5, C6, C7, | | | | |
| | C8, C9 | | | | |
| OC31 | C3b, Properdin | Heavy chain | H-26 | WO2015099838 | 20260 |
| | (factor P), | | | SEQ ID NO: 69 | |
| | Factors Ba and | | | | |
| | Bb, C5, C6, C7, | | | | |
| | C8, C9 | | | | |
| OC32 | C3b, Properdin | Heavy chain | H-27 | WO2015099838 | 20261 |
| | (factor P), | | | SEQ ID NO: 70 | |
| | Factors Ba and | | | | |
| | Bb, C5, C6, C7, | | | | |
| | C8, C9 | | | | |
| OC33 | C3b, Properdin | Heavy chain | H-28 | WO2015099838 | 20262 |
| | (factor P), | | | SEQ ID NO: 71 | |
| | Factors Ba and | | | | |
| | Bb, C5, C6, C7, | | | | |
| | C8, C9 | | | | |
| OC34 | C3b, Properdin | Heavy chain | H-29 | WO2015099838 | 20263 |
| | (factor P), | | | SEQ ID NO: 72 | |
| | Factors Ba and | | | | |
| | Bb, C5, C6, C7, | | | | |
| | C8, C9 | | | | |
| OC35 | C3b, Properdin | Heavy chain | H-30 | WO2015099838 | 20264 |
| | (factor P), | | | SEQ ID NO: 73 | |
| | Factors Ba and | | | | |
| | Bb, C5, C6, C7, | | | | |
| | C8, C9 | | | | |
| OC36 | C3b, Properdin | Heavy chain | H-31 | WO2015099838 | 20265 |
| | (factor P), | | | SEQ ID NO: 74 | |
| | Factors Ba and | | | | |
| | Bb, C5, C6, C7, | | | | |
| | C8, C9 | | | | |
| OC37 | C3b, Properdin | Heavy chain | H-32 | WO2015099838 | 20266 |
| | (factor P), | | | SEQ ID NO: 75 | |
| | Factors Ba and | | | | |
| | Bb, C5, C6, C7, | | | | |
| | C8, C9 | | | | |
| OC38 | C3b, Properdin | Heavy chain | H-33 | WO2015099838 | 20267 |
| | (factor P), | | | SEQ ID NO: 76 | |
| | Factors Ba and | | | | |
| | Bb, C5, C6, C7, | | | | |
| | C8, C9 | | | | |
| OC39 | C3b, Properdin | Heavy chain | H-34 | WO2015099838 | 20268 |
| | (factor P), | | | SEQ ID NO: 77 | |
| | Factors Ba and | | | | |
| | Bb, C5, C6, C7, | | | | |
| | C8, C9 | | | | |
| OC40 | C3b, Properdin | Heavy chain | H-35 | WO2015099838 | 20269 |
| | (factor P), | | | SEQ ID NO: 78 | |
| | Factors Ba and | | | | |
| | Bb, C5, C6, C7, | | | | |
| | C8, C9 | | | | |
| OC41 | C3b, Properdin | Heavy chain | H-36 | WO2015099838 | 20270 |
| | (factor P), | | | SEQ ID NO: 79 | |
| | Factors Ba and | | | | |
| | Bb, C5, C6, C7, | | | | |
| | C8, C9 | | | | |
| OC42 | C3b, Properdin | Heavy chain | H-37 | WO2015099838 | 20271 |
| | (factor P), | | | SEQ ID NO: 80 | |
| | Factors Ba and | | | | |
| | Bb, C5, C6, C7, | | | | |
| | C8, C9 | | | | |
| OC43 | C3b, Properdin | Heavy chain | H-38 | WO2015099838 | 20272 |
| | (factor P), | | | SEQ ID NO: 81 | |
| | Factors Ba and | | | | |
| | Bb, C5, C6, C7, | | | | |
| | C8, C9 | | | | |
| OC44 | C3b, Properdin | Heavy chain | H-39 | WO2015099838 | 20273 |
| | (factor P), | | | SEQ ID NO: 82 | |
| | Factors Ba and | | | | |
| | Bb, C5, C6, C7, | | | | |
| | C8, C9 | | | | |
| OC45 | C3b, Properdin | Heavy chain | H-40 | WO2015099838 | 20274 |
| | (factor P), | | | SEQ ID NO: 83 | |
| | Factors Ba and | | | | |
| | Bb, C5, C6, C7, | | | | |
| | C8, C9 | | | | |
| OC46 | C3b, Properdin | Heavy chain | H-41 | WO2015099838 | 20275 |
| | (factor P), | | | SEQ ID NO: 84 | |
| | Factors Ba and | | | | |
| | Bb, C5, C6, C7, | | | | |
| | C8, C9 | | | | |
| OC47 | C3b, Properdin | Heavy chain | H-42 | WO2015099838 | 20276 |
| | (factor P), | | | SEQ ID NO: 85 | |
| | Factors Ba and | | | | |
| | Bb, C5, C6, C7, | | | | |
| | C8, C9 | | | | |
| OC48 | C3b, Properdin | Heavy chain | H-43 | WO2015099838 | 20277 |
| | (factor P), | | | SEQ ID NO: 86 | |
| | Factors Ba and | | | | |
| | Bb, C5, C6, C7, | | | | |
| | C8, C9 | | | | |
| OC49 | C3b, Properdin | Heavy chain | H-1 | WO2015099838 | 20278 |
| | (factor P), | | | SEQ ID NO: 44 | |
| | Factors Ba and | | | | |
| | Bb, C5, C6, C7, | | | | |
| | C8, C9 | | | | |
| OC50 | C3b, Properdin | Heavy chain | H-2 | WO2015099838 | 20279 |
| | (factor P), | | | SEQ ID NO: 45 | |
| | Factors Ba and | | | | |
| | Bb, C5, C6, C7, | | | | |
| | C8, C9 | | | | |
| OC51 | C3b, Properdin | Heavy chain | H-3 | WO2015099838 | 20280 |
| | (factor P), | | | SEQ ID NO: 46 | |
| | Factors Ba and | | | | |
| | Bb, C5, C6, C7, | | | | |
| | C8, C9 | | | | |
| OC52 | C3b, Properdin | Heavy chain | H-4 | WO2015099838 | 20281 |
| | (factor P), | | | SEQ ID NO: 47 | |
| | Factors Ba and | | | | |
| | Bb, C5, C6, C7, | | | | |
| | C8, C9 | | | | |
| OC53 | C3b, Properdin | Heavy chain | H-5 | WO2015099838 | 20282 |
| | (factor P), | | | SEQ ID NO: 48 | |
| | Factors Ba and | | | | |
| | Bb, C5, C6, C7, | | | | |
| | C8, C9 | | | | |
| OC54 | C5 | Heavy chain | NVS808 | US20150158936 | 20283 |
| | | | | SEQ ID NO: 107 | |
| OC55 | C5 | Heavy chain | NVS806 | US20150158936 | 20284 |
| | | | | SEQ ID NO: 121 | |
| OC56 | C5 | Heavy chain | NVS804 | US20150158936 | 20285 |
| | | | | SEQ ID NO: 135 | |
| OC57 | C5 | Heavy chain | NVS809 | US20150158936 | 20286 |
| | | | | SEQ ID NO: 149 | |
| OC58 | C5 | Heavy chain | NVS805 | US20150158936 | 20287 |
| | | | | SEQ ID NO: 163 | |
| OC59 | C5 | Heavy chain | NVS962-S | US20150158936 | 20288 |
| | | | | SEQ ID NO: 177 | |
| OC60 | C5 | Heavy chain | NVS962-Q | US20150158936 | 20289 |
| | | | | SEQ ID NO: 191 | |
| OC61 | C5 | Heavy chain | NVS962-S31A | US20150158936 | 20290 |
| | | | | SEQ ID NO: 205 | |
| OC62 | C5 | Heavy chain | NVS962-G | US20150158936 | 20291 |
| | | | | SEQ ID NO: 219 | |
| OC63 | C5 | Heavy chain | NVS963 | US20150158936 | 20292 |
| | | | | SEQ ID NO: 23 | |
| OC64 | C5 | Heavy chain | NVS962-T | US20150158936 | 20293 |
| | | | | SEQ ID NO: 233 | |
| OC65 | C5 | Heavy chain | NVS965-T | US20150158936 | 20294 |
| | | | | SEQ ID NO: 247 | |
| OC66 | C5 | Heavy chain | NVS965-Q | US20150158936 | 20295 |
| | | | | SEQ ID NO: 261 | |
| OC67 | C5 | Heavy chain | NVS965-S | US20150158936 | 20296 |
| | | | | SEQ ID NO: 275 | |
| OC68 | C5 | Heavy chain | NVS964 | US20150158936 | 20297 |
| | | | | SEQ ID NO: 37 | |
| OC69 | C5 | Heavy chain | Antibody 8109 | US20150158936 | 20298 |
| | | | | SEQ ID NO: 418 | |
| OC70 | C5 | Heavy chain | Antibody 8110 | US20150158936 | 20299 |
| | | | | SEQ ID NO: 434 | |
| OC71 | C5 | Heavy chain | Antibody 8111 | US20150158936 | 20300 |
| | | | | SEQ ID NO: 449 | |
| OC72 | C5 | Heavy chain | Antibody 8113 | US20150158936 | 20301 |
| | | | | SEQ ID NO: 462 | |
| OC73 | C5 | Heavy chain | Antibody 8114 | US20150158936 | 20302 |
| | | | | SEQ ID NO: 478 | |
| OC74 | C5 | Heavy chain | NVS966 | US20150158936 | 20303 |
| | | | | SEQ ID NO: 51 | |
| OC75 | C5 | Heavy chain | NVS965 | US20150158936 | 20304 |
| | | | | SEQ ID NO: 65 | |
| OC76 | C5 | Heavy chain | NVS967 | US20150158936 | 20305 |
| | | | | SEQ ID NO: 79 | |
| OC77 | C5 | Heavy chain | NVS962 | US20150158936 | 20306 |
| | | | | SEQ ID NO: 9 | |
| OC78 | C5 | Heavy chain | NVS807 | US20150158936 | 20307 |
| | | | | SEQ ID NO: 93 | |
| OC79 | C5 | Heavy chain | H5 | US20150239966 | 20308 |
| | | | | SEQ ID NO: 10 | |
| OC80 | C5 | Heavy chain | H6 | US20150239966 | 20309 |
| | | | | SEQ ID NO: 12 | |
| OC81 | C5 | Heavy chain | H1 | US20150239966 | 20310 |
| | | | | SEQ ID NO: 2 | |
| OC82 | C5 | Heavy chain | H2 | US20150239966 | 20311 |
| | | | | SEQ ID NO: 4 | |
| OC83 | C5 | Heavy chain | H3 | US20150239966 | 20312 |
| | | | | SEQ ID NO: 6 | |
| OC84 | C5 | Heavy chain | H4 | US20150239966 | 20313 |
| | | | | SEQ ID NO: 8 | |
| OC85 | C5 | Heavy chain | Tesidolumab, | U.S. Pat. No. 8,241,628 | 20314 |
| | | | “LFG 316, | SEQ ID NO: 9 | |
| | | | LFG-316, | | |
| | | | LFG316” | | |
| OC86 | C5 | Heavy chain | | U.S. Pat. No. 9,133,269 | 20315 |
| | | | | SEQ ID NO: 1 | |
| OC87 | C5 | Heavy chain | | U.S. Pat. No. 9,133,269 | 20316 |
| | | | | SEQ ID NO: 2 | |
| OC88 | C5 | Heavy chain | | U.S. Pat. No. 9,133,269 | 20317 |
| | | | | SEQ ID NO: 27 | |
| OC89 | C5 | Heavy chain | | U.S. Pat. No. 9,133,269 | 20318 |
| | | | | SEQ ID NO: 3 | |
| OC90 | C5 | Heavy chain | | U.S. Pat. No. 9,133,269 | 20319 |
| | | | | SEQ ID NO: 4 | |
| OC91 | C5 | Heavy chain | | U.S. Pat. No. 9,133,269 | 20320 |
| | | | | SEQ ID NO: 5 | |
| OC92 | C5a | Heavy chain | BNJ364 | US20130224187 | 20321 |
| | | | | SEQ ID NO: 25 | |
| OC93 | C5a | Heavy chain | BNJ367, | US20130224187 | 20322 |
| | | | BNJ371, | SEQ ID NO: 33 | |
| | | | BNJ378 | | |
| OC94 | C5a | Heavy chain | BNJ366 | US20130224187 | 20323 |
| | | | | SEQ ID NO: 44 | |
| OC95 | CA 125 | Heavy chain | Sofituzumab | U.S. Pat. No. 7,723,485 | 20324 |
| | (MUC16) | | vedotin, | SEQ ID NO: 1 | |
| | | | DMUC5754A | | |
| | | | (conjugate), | | |
| | | | MMUC1206A | | |
| | | | (nonconjugate) | | |
| OC96 | CGRP | Heavy chain | Ab4 | US20120294802 | 20325 |
| | | | | SEQ ID NO: 34 | |
| OC97 | CGRP | Heavy chain | Ab1 | US20120294802 | 20326 |
| | | | | SEQ ID NO: 4 | |
| OC98 | CGRP | Heavy chain | Ab5 | US20120294802 | 20327 |
| | | | | SEQ ID NO: 44 | |
| OC99 | CGRP | Heavy chain | Ab6 | US20120294802 | 20328 |
| | | | | SEQ ID NO: 54 | |
| OC100 | CGRP | Heavy chain | Ab7 | US20120294802 | 20329 |
| | | | | SEQ ID NO: 64 | |
| OC101 | CGRP | Heavy chain | Ab8 | US20120294802 | 20330 |
| | | | | SEQ ID NO: 74 | |
| OC102 | CGRP | Heavy chain | Ab9 | US20120294802 | 20331 |
| | | | | SEQ ID NO: 84 | |
| OC103 | CGRP | Heavy chain | Ab10 | US20120294802 | 20332 |
| | | | | SEQ ID NO: 94 | |
| OC104 | CGRP | Heavy chain | Ab11 | US20120294802 | 20333 |
| | | | | SEQ ID NO: 104 | |
| OC105 | CGRP | Heavy chain | Ab12 | US20120294802 | 20334 |
| | | | | SEQ ID NO: 114 | |
| OC106 | CGRP | Heavy chain | Ab13 | US20120294802 | 20335 |
| | | | | SEQ ID NO: 124 | |
| OC107 | CGRP | Heavy chain | Ab14 | US20120294802 | 20336 |
| | | | | SEQ ID NO: 134 | |
| OC108 | CGRP | Heavy chain | Ab2 | US20120294802 | 20337 |
| | | | | SEQ ID NO: 14 | |
| OC109 | CGRP | Heavy chain | Ab3 | US20120294802 | 20338 |
| | | | | SEQ ID NO: 24 | |
| OC110 | Factor D | Heavy chain | Fab 238 | WO2009134711 | 20339 |
| | | | | SEQ ID NO: 52 | |
| OC111 | Factor D, | Heavy Chain | Lampalizumab, | U.S. Pat. No. 8,273,352 | 20340 |
| | humanized | | | SEQ ID NO: 62 | |
| | IgG1 | | | | |
| OC112 | platelet-derived | Heavy chain | Rinucumab, | | 20341 |
| | growth factor | | REGN2176 | | |
| | receptor beta | | | | |
| | PDGFRB | | | | |
| OC113 | S1P4 | Heavy chain | | WO2015057939 | 20342 |
| | | | | SEQ ID NO: 39 | |
| OC114 | VEGF, C5, | Heavy chain | NVS73 | US20140186350 | 20343 |
| | Factor P, Factor | | | SEQ ID 113 | |
| | D, EPO, EPOR, | | | | |
| | IL-1β, IL-17A, | | | | |
| | Il-10, TNFα, or | | | | |
| | FGFR2 | | | | |
| OC115 | VEGF, C5, | Heavy chain | NVS73T | US20140186350 | 20344 |
| | Factor P, Factor | | | SEQ ID 115 | |
| | D, EPO, EPOR, | | | | |
| | IL-1β, IL-17A, | | | | |
| | Il-10, TNFα, or | | | | |
| | FGFR2 | | | | |
| OC116 | VEGF, C5, | Heavy chain | NVS75 | US20140186350 | 20345 |
| | Factor P, Factor | | | SEQ ID 194 | |
| | D, EPO, EPOR, | | | | |
| | IL-1β, IL-17A, | | | | |
| | Il-10, TNFα, or | | | | |
| | FGFR2 | | | | |
| OC117 | VEGF, C5, | Heavy chain | NVS74T, | US20140186350 | 20346 |
| | Factor P, Factor | | NCS75T | SEQ ID 196 | |
| | D, EPO, EPOR, | | | | |
| | IL-1β, IL-17A, | | | | |
| | Il-10, TNFα, or | | | | |
| | FGFR2 | | | | |
| OC118 | VEGF, C5, | Heavy chain | NVS1 | US20140186350 | 20347 |
| | Factor P, Factor | | | SEQ ID 21 | |
| | D, EPO, EPOR, | | | | |
| | IL-1β, IL-17A, | | | | |
| | Il-10, TNFα, or | | | | |
| | FGFR2 | | | | |
| OC119 | VEGF, C5, | Heavy chain | NVS2 | US20140186350 | 20348 |
| | Factor P, Factor | | | SEQ ID 23 | |
| | D, EPO, EPOR, | | | | |
| | IL-1β, IL-17A, | | | | |
| | Il-10, TNFα, or | | | | |
| | FGFR2 | | | | |
| OC120 | VEGF, C5, | Heavy chain | NVS3 | US20140186350 | 20349 |
| | Factor P, Factor | | | SEQ ID 25 | |
| | D, EPO, EPOR, | | | | |
| | IL-1β, IL-17A, | | | | |
| | Il-10, TNFα, or | | | | |
| | FGFR2 | | | | |
| OC121 | VEGF, C5, | Heavy chain | NVS36 | US20140186350 | 20350 |
| | Factor P, Factor | | | SEQ ID 27 | |
| | D, EPO, EPOR, | | | | |
| | IL-1β, IL-17A, | | | | |
| | Il-10, TNFα, or | | | | |
| | FGFR2 | | | | |
| OC122 | VEGF, C5, | Heavy chain | NVS37 | US20140186350 | 20351 |
| | Factor P, Factor | | | SEQ ID 29 | |
| | D, EPO, EPOR, | | | | |
| | IL-1β, IL-17A, | | | | |
| | Il-10, TNFα, or | | | | |
| | FGFR2 | | | | |
| OC123 | VEGF, C5, | Heavy chain | NVS70 | US20140186350 | 20352 |
| | Factor P, Factor | | | SEQ ID 42 | |
| | D, EPO, EPOR, | | | | |
| | IL-1β, IL-17A, | | | | |
| | Il-10, TNFα, or | | | | |
| | FGFR2 | | | | |
| OC124 | VEGF, C5, | Heavy chain | NVS70T | US20140186350 | 20353 |
| | Factor P, Factor | | | SEQ ID 44 | |
| | D, EPO, EPOR, | | | | |
| | IL-1β, IL-17A, | | | | |
| | Il-10, TNFα, or | | | | |
| | FGFR2 | | | | |
| OC125 | VEGF, C5, | Heavy chain | NVS71 | US20140186350 | 20354 |
| | Factor P, Factor | | | SEQ ID 61 | |
| | D, EPO, EPOR, | | | | |
| | IL-1β, IL-17A, | | | | |
| | Il-10, TNFα, or | | | | |
| | FGFR2 | | | | |
| OC126 | VEGF, C5, | Heavy chain | NVS71T | US20140186350 | 20355 |
| | Factor P, Factor | | | SEQ ID 63 | |
| | D, EPO, EPOR, | | | | |
| | IL-1β, IL-17A, | | | | |
| | Il-10, TNFα, or | | | | |
| | FGFR2 | | | | |
| OC127 | VEGF, C5, | Heavy chain | NVS72 | US20140186350 | 20356 |
| | Factor P, Factor | | | SEQ ID 83 | |
| | D, EPO, EPOR, | | | | |
| | IL-1β, IL-17A, | | | | |
| | Il-10, TNFα, or | | | | |
| | FGFR2 | | | | |
| OC128 | VEGF, C5, | Heavy chain | NVS72T | US20140186350 | 20357 |
| | Factor P, Factor | | | SEQ ID 85 | |
| | D, EPO, EPOR, | | | | |
| | IL-1β, IL-17A, | | | | |
| | Il-10, TNFα, or | | | | |
| | FGFR2 | | | | |
| OC129 | VEGF, C5, | Heavy chain | NVS4, NVS1j | US20140186350 | 20358 |
| | Factor P, Factor | | | SEQ ID 9 | |
| | D, EPO, EPOR, | | | | |
| | IL-1β, IL-17A, | | | | |
| | Il-10, TNFα, or | | | | |
| | FGFR2 | | | | |
| OC130 | VEGF, C5, | Heavy chain | NVS81 | US20140186350 | 20359 |
| | Factor P, Factor | | | SEQ ID 157 | |
| | D, EPO, EPOR, | | | | |
| | IL-1β, IL-17A, | | | | |
| | Il-10, TNFα, or | | | | |
| | FGFR2 | | | | |
| OC131 | VEGF, C5, | Heavy chain | NVS81T | US20140186350 | 20360 |
| | Factor P, Factor | | | SEQ ID 159 | |
| | D, EPO, EPOR, | | | | |
| | IL-1β, IL-17A, | | | | |
| | Il-10, TNFα, or | | | | |
| | FGFR2 | | | | |
| OC132 | VEGF, C5. | Heavy chain | NVS82 | US20140186350 | 20361 |
| | Factor P, Factor | | | SEQ ID 161 | |
| | D, EPO, EPOR, | | | | |
| | IL-1β, IL-17A, | | | | |
| | Il-10, TNFα, or | | | | |
| | FGFR2 | | | | |
| OC133 | VEGF, C5, | Heavy chain | NVS82T | US20140186350 | 20362 |
| | Factor P, Factor | | | SEQ ID 163 | |
| | D, EPO, EPOR, | | | | |
| | IL-1β, IL-17A, | | | | |
| | Il-10, TNFα, or | | | | |
| | FGFR2 | | | | |
| OC134 | VECF, C5, | Heavy chain | NVS1b | US20140186350 | 20363 |
| | Factor P, Factor | | | SEQ ID 171 | |
| | D, EPO, EPOR, | | | | |
| | IL-1β, IL-17A, | | | | |
| | Il-10, TNFα, or | | | | |
| | FGFR2 | | | | |
| OC135 | VEGF, C5, | Heavy chain | NVS1c | US20140186350 | 20364 |
| | Factor P, Factor | | | SEQ ID 173 | |
| | D, EPO, EPOR, | | | | |
| | IL-1β, IL-17A, | | | | |
| | Il-10, TNFα, or | | | | |
| | FGFR2 | | | | |
| OC136 | VEGF, C5, | Heavy chain | NVS1d | US20140186350 | 20365 |
| | Factor P, Factor | | | SEQ ID 175 | |
| | D, EPO, EPOR, | | | | |
| | IL-1β, IL-17A, | | | | |
| | Il-10, TNFα, or | | | | |
| | FGFR2 | | | | |
| OC137 | VEGF, C5, | Heavy chain | NVS1e | US20140186350 | 20366 |
| | Factor P, Factor | | | SEQ ID 177 | |
| | D, EPO, EPOR, | | | | |
| | IL-1β, IL-17A, | | | | |
| | Il-10, TNFα, or | | | | |
| | FGFR2 | | | | |
| OC138 | VEGF, C5, | Heavy chain | NVS1f | US20140186350 | 20367 |
| | Factor P, Factor | | | SEQ ID 179 | |
| | D, EPO, EPOR, | | | | |
| | IL-1β, IL-17A, | | | | |
| | Il-10, TNFα, or | | | | |
| | FGFR2 | | | | |
| OC139 | VEGF, C5, | Heavy chain | NVS1g | US20140186350 | 20368 |
| | Factor P, Factor | | | SEQ ID 181 | |
| | D, EPO, EPOR, | | | | |
| | IL-1β, IL-17A, | | | | |
| | Il-10, TNFα, or | | | | |
| | FGFR2 | | | | |
| OC140 | VEGF, C5, | Heavy chain | NVS1h | US20140186350 | 20369 |
| | Factor P, Factor | | | SEQ ID 183 | |
| | D, EPO, EPOR, | | | | |
| | IL-1β, IL-17A, | | | | |
| | Il-10, TNFα, or | | | | |
| | FGFR2 | | | | |
| OC141 | sphingosine-1- | Heavy chain | Sonepcizumab, | | 20370 |
| | phosphate | full | S1P-LT1011 | | |
| OC142 | sphingosine-1- | Heavy chain | Sonepcizumab, | | 20371 |
| | phosphate | variable | S1P-LT1009 | | |
| OC143 | Factor D | Heavy chain | Fab 238 | WO2009134711 | 20372 |
| | | variable region | | SEQ ID NO: 18 | |
| OC144 | Factor D | Heavy chain | Fab 238-1 | WO2009134711 | 20373 |
| | | variable region | | SEQ ID NO: 19 | |
| OC145 | Factor D | Heavy chain | Humanized | WO2009134711 | 20374 |
| | | variable region | Clone #111 | SEQ ID NO: 2 | |
| OC146 | Factor D | Heavy chain | Fab 238-2 | WO2009134711 | 20375 |
| | | variable region | | SEQ ID NO: 20 | |
| OC147 | Factor D | Heavy chain | Fab 238-3 | WO2009134711 | 20376 |
| | | variable region | | SEQ ID NO: 21 | |
| OC148 | Factor D | Heavy chain | Fab 238-4 | WO2009134711 | 20377 |
| | | variable region | | SEQ ID NO: 22 | |
| OC149 | Factor D | Heavy chain | Fab 238-5 | WO2009134711 | 20378 |
| | | variable region | | SEQ ID NO: 23 | |
| OC150 | Factor D | Heavy chain | Fab 238-6 | WO2009134711 | 20379 |
| | | variable region | | SEQ ID NO: 24 | |
| OC151 | Factor D | Heavy chain | Fab 238-7 | WO2009134711 | 20380 |
| | | variable region | | SEQ ID NO: 25 | |
| OC152 | Factor D | Heavy chain | Fab 238-8 | WO2009134711 | 20381 |
| | | variable region | | SEQ ID NO: 26 | |
| OC153 | Factor D | Heavy chain | Fab 238-9 | WO2009134711 | 20382 |
| | | variable region | | SEQ ID NO: 27 | |
| OC154 | Factor D | Heavy chain | Fab 238-10 | WO2009134711 | 20383 |
| | | variable region | | SEQ ID NO: 28 | |
| OC155 | Factor D | Heavy chain | Fab 238-11 | WO2009134711 | 20384 |
| | | variable region | | SEQ ID NO: 29 | |
| OC156 | Factor D | Heavy chain | L243 | WO2009134711 | 20385 |
| | | variable region | | SEQ ID NO: 34 | |
| OC157 | Factor D | Heavy chain | humanized | WO2009134711 | 20386 |
| | | variable region | L243 | SEQ ID NO: 38 | |
| OC158 | LPG | Heavy chain | #7 | U.S. Pat. No. 8,591,902 | 20387 |
| | (lysophosphati- | variable region | | SEQ ID NO: 18 | |
| | dylglucoside) | | | | |
| OC159 | LPG | Heavy chain | #15 | U.S. Pat. No. 8,591,902 | 20388 |
| | (lysophosphati- | variable region | | SEQ ID NO: 8 | |
| | dylglucoside) | | | | |
| OC160 | PDGFR-beta | Heavy chain | 3373N | US20140193402 | 20389 |
| | | variable region | | SEQ ID 114 | |
| OC161 | PDGFR-beta | Heavy chain | 3374N | US20140193402 | 20390 |
| | | variable region | | SEQ ID 130 | |
| OC162 | PDGFR-beta | Heavy chain | 3094P | US20140193402 | 20391 |
| | | variable region | | SEQ ID 146 | |
| OC163 | PDGFR-beta | Heavy chain | 3095S | US20140193402 | 20392 |
| | | variable region | | SEQ ID 162 | |
| OC164 | PDGFR-beta | Heavy chain | 3096S | US20140193402 | 20393 |
| | | variable region | | SEQ ID 178 | |
| OC165 | PDGFR-beta | Heavy chain | 3305N | US20140193402 | 20394 |
| | | variable region | | SEQ ID 18 | |
| OC166 | PDGFR-beta | Heavy chain | 3097S | US20140193402 | 20395 |
| | | variable region | | SEQ ID 194 | |
| OC167 | PDGFR-beta | Heavy chain | 3299N | US20140193402 | 20396 |
| | | variable region | | SEQ ID 2 | |
| OC168 | PDGFR-beta | Heavy chain | 3098S | US20140193402 | 20397 |
| | | variable region | | SEQ ID 210 | |
| OC169 | PDGFR-beta | Heavy chain | 3099S | US20140193402 | 20398 |
| | | variable region | | SEQ ID 226 | |
| OC170 | PDGFR-beta | Heavy chain | 3102S | US20140193402 | 20399 |
| | | variable region | | SEQ ID 242 | |
| OC171 | PDGFR-beta | Heavy chain | 3103S | US20140193402 | 20400 |
| | | variable region | | SEQ ID 258 | |
| OC172 | PDGFR-beta | Heavy chain | 3104S | US20140193402 | 20401 |
| | | variable region | | SEQ ID 274 | |
| OC173 | PDGFR-beta | Heavy chain | 3105S | US20140193402 | 20402 |
| | | variable region | | SEQ ID 290 | |
| OC174 | PDGFR-beta | Heavy chain | 3106S | US20140193402 | 20403 |
| | | variable region | | SEQ ID 306 | |
| OC175 | PDGFR-beta | Heavy chain | 3107S | US20140193402 | 20404 |
| | | variable region | | SEQ ID 322 | |
| OC176 | PDGFR-beta | Heavy chain | 3310N | US20140193402 | 20405 |
| | | variable region | | SEQ ID 34 | |
| OC177 | PDGFR-beta | Heavy chain | 3361N | US20140193402 | 20406 |
| | | variable region | | SEQ ID 50 | |
| OC178 | PDGFR-beta | Heavy chain | 3363N | US20140193402 | 20407 |
| | | variable region | | SEQ ID 66 | |
| OC179 | PDGFR-beta | Heavy chain | 3365N | US20140193402 | 20408 |
| | | variable region | | SEQ ID 82 | |
| OC180 | PDGFR-beta | Heavy chain | 3368N | US20140193402 | 20409 |
| | | variable region | | SEQ ID 98 | |
| OC181 | PDGFRβ/VEGF-A | Heavy chain | Cluster # 1322 | US20110177074 | 20410 |
| | | variable region | | SEQ ID NO: 100 | |
| OC182 | PDGFRβ/VEGF-A | Heavy chain | Cluster # 1323 | US20110177074 | 20411 |
| | | variable region | | SEQ ID NO: 104 | |
| OC183 | PDGFRβ/VEGF-A | Heavy chain | Cluster # 1330 | US20110177074 | 20412 |
| | | variable region | | SEQ ID NO: 108 | |
| OC184 | PDGFRβ/VEGF-A | Heavy chain | Cluster # 1334 | US20110177074 | 20413 |
| | | variable region | | SEQ ID NO: 112 | |
| OC185 | PDGFRβ/VEGF-A | Heavy chain | Cluster # 1345 | US20110177074 | 20414 |
| | | variable region | | SEQ ID NO: 116 | |
| OC186 | PDGFRβ/VEGF-A | Heavy chain | Cluster # 600 | US20110177074 | 20415 |
| | | variable region | | SEQ ID NO: 12 | |
| OC187 | PDGFRβ/VEGF-A | Heavy chain | Cluster # 1346 | US20110177074 | 20416 |
| | | variable region | | SEQ ID NO: 120 | |
| OC188 | PDGFRβ/VEGF-A | Heavy chain | Cluster # 1359 | US20110177074 | 20417 |
| | | variable region | | SEQ ID NO: 124 | |
| OC189 | PDGFRβ/VEGF-A | Heavy chain | Cluster # 1365 | US20110177074 | 20418 |
| | | variable region | | SEQ ID NO: 128 | |
| OC190 | PDGFRβ/VEGF-A | Heavy chain | Cluster # 1402 | US20110177074 | 20419 |
| | | variable region | | SEQ ID NO: 132 | |
| OC191 | PDGFRβ/VEGF-A | Heavy chain | Cluster # 1515 | US20110177074 | 20420 |
| | | variable region | | SEQ ID NO: 136 | |
| OC192 | PDGFRβ/VEGF-A | Heavy chain | Cluster # 1531 | US20110177074 | 20421 |
| | | variable region | | SEQ ID NO: 140 | |
| OC193 | PDGFRβ/VEGF-A | Heavy chain | Cluster # 1535 | US20110177074 | 20422 |
| | | variable region | | SEQ ID NO: 144 | |
| OC194 | PDGFRβ/VEGF-A | Heavy chain | Cluster # 1541 | US20110177074 | 20423 |
| | | variable region | | SEQ ID NO: 148 | |
| OC195 | PDGFRβ/VEGF-A | Heavy chain | Cluster # 1550 | US20110177074 | 20424 |
| | | variable region | | SEQ ID NO: 152 | |
| OC196 | PDGFRβ/VEGF-A | Heavy chain | Cluster # 1564 | US20110177074 | 20425 |
| | | variable region | | SEQ ID NO: 156 | |
| OC197 | PDGFRβ/VEGF-A | Heavy chain | Cluster # 607 | US20110177074 | 20426 |
| | | variable region | | SEQ ID NO: 16 | |
| OC198 | PDGFRβ/VEGF-A | Heavy chain | Cluster # 1601 | US20110177074 | 20427 |
| | | variable region | | SEQ ID NO: 160 | |
| OC199 | PDGFRβ/VEGF-A | Heavy chain | Cluster # 1629 | US20110177074 | 20428 |
| | | variable region | | SEQ ID NO: 164 | |
| OC200 | PDGFRβ/VEGF-A | Heavy chain | Cluster # 635 | US20110177074 | 20429 |
| | | variable region | | SEQ ID NO: 168 | |
| OC201 | PDGFRβ/VEGF-A | Heavy chain | Cluster # 636 | US20110177074 | 20430 |
| | | variable region | | SEQ ID NO: 172 | |
| OC202 | PDGFRβ/VEGF-A | Heavy chain | Cluster # 638 | US20110177074 | 20431 |
| | | variable region | | SEQ ID NO: 176 | |
| OC203 | PDGFRβ/VEGF-A | Heavy chain | Cluster # 656 | US20110177074 | 20432 |
| | | variable region | | SEQ ID NO: 180 | |
| OC204 | PDGFRβ/VEGF-A | Heavy chain | Cluster # 665 | US20110177074 | 20433 |
| | | variable region | | SEQ ID NO: 184 | |
| OC205 | PDGFRβ/VEGF-A | Heavy chain | Cluster # 668 | US20110177074 | 20434 |
| | | variable region | | SEQ ID NO: 188 | |
| OC206 | PDGFRβ/VEGF-A | Heavy drain | Cluster # 669 | US20110177074 | 20435 |
| | | variable region | | SEQ ID NO: 192 | |
| OC207 | PDGFRβ/VEGF-A | Heavy chain | Cluster # 679 | US20110177074 | 20436 |
| | | variable region | | SEQ ID NO: 196 | |
| OC208 | PDGFRβ/VEGF-A | Heavy chain | Cluster # 613 | US20110177074 | 20437 |
| | | variable region | | SEQ ID NO: 20 | |
| OC209 | PDGFRβ/VEGF-A | Heavy chain | Cluster # 695 | US20110177074 | 20438 |
| | | variable region | | SEQ ID NO: 200 | |
| OC210 | PDGFRβ/VEGF-A | Heavy chain | Cluster # 709 | US20110177074 | 20439 |
| | | variable region | | SEQ ID NO: 204 | |
| OC211 | PDGFRβ/VEGF-A | Heavy chain | Cluster # 710 | US20110177074 | 20440 |
| | | variable region | | SEQ ID NO: 208 | |
| OC212 | PDGFRβ/VEGF-A | Heavy chain | Cluster # 741 | US20110177074 | 20441 |
| | | variable region | | SEQ ID NO: 212 | |
| OC213 | PDGFRβ/VEGF-A | Heavy chain | Cluster # 752 | US20110177074 | 20442 |
| | | variable region | | SEQ ID NO: 216 | |
| OC214 | PDGFRβ/VEGF-A | Heavy chain | Cluster # 772 | US20110177074 | 20443 |
| | | variable region | | SEQ ID NO: 220 | |
| OC215 | PDGFRβ/VEGF-A | Heavy chain | Cluster # 779 | US20110177074 | 20444 |
| | | variable region | | SEQ ID NO: 224 | |
| OC216 | PDGFRβ/VEGF-A | Heavy chain | Cluster # 799 | US20110177074 | 20445 |
| | | variable region | | SEQ ID NO: 228 | |
| OC217 | PDGFRβ/VEGF-A | Heavy chain | Cluster # 830 | US20110177074 | 20446 |
| | | variable region | | SEQ ID NO: 232 | |
| OC218 | PDGFRβ/VEGF-A | Heavy chain | Cluster # 844 | US20110177074 | 20447 |
| | | variable region | | SEQ ID NO: 236 | |
| OC219 | PDGFRβ/VEGF-A | Heavy chain | Cluster # 941 | US20110177074 | 20448 |
| | | variable region | | SEQ ID NO: 24 | |
| OC220 | PDGFRβ/VEGF-A | Heavy chain | Cluster # 847 | US20110177074 | 20449 |
| | | variable region | | SEQ ID NO: 240 | |
| OC221 | PDGFRβ/VEGF-A | Heavy chain | Cluster # 868 | US20110177074 | 20450 |
| | | variable region | | SEQ ID NO: 244 | |
| OC222 | PDGFRβ/VEGF-A | Heavy chain | Cluster # 870 | US20110177074 | 20451 |
| | | variable region | | SEQ ID NO: 248 | |
| OC223 | PDGFRβ/VEGF-A | Heavy chain | Cluster # 883 | US20110177074 | 20452 |
| | | variable region | | SEQ ID NO: 252 | |
| OC224 | PDGFRβ/VEGF-A | Heavy chain | Cluster # 887 | US20110177074 | 20453 |
| | | variable region | | SEQ ID NO: 256 | |
| OC225 | PDGFRβ/VEGF-A | Heavy chain | Cluster # 901 | US20110177074 | 20454 |
| | | variable region | | SEQ ID NO: 260 | |
| OC226 | PDGFRβ/VEGF-A | Heavy chain | Cluster # 905 | US20110177074 | 20455 |
| | | variable region | | SEQ ID NO: 264 | |
| OC227 | PDGFRβ/VEGF-A | Heavy chain | Cluster # 909 | US20110177074 | 20456 |
| | | variable region | | SEQ ID NO: 268 | |
| OC228 | PDGFRβ/VEGF-A | Heavy chain | Cluster # 928 | US20110177074 | 20457 |
| | | variable region | | SEQ ID NO: 272 | |
| OC229 | PDGFRβ/VEGF-A | Heavy chain | Cluster # 1036 | US20110177074 | 20458 |
| | | variable region | | SEQ ID NO: 276 | |
| OC230 | PDGFRβ/VEGF-A | Heavy chain | Cluster # 946 | US20110177074 | 20459 |
| | | variable region | | SEQ ID NO: 28 | |
| OC231 | PDGFRβ/VEGF-A | Heavy chain | Cluster # 1039 | US20110177074 | 20460 |
| | | variable region | | SEQ ID NO: 280 | |
| OC232 | PDGFRβ/VEGF-A | Heavy chain | Cluster # 1040 | US20110177074 | 20461 |
| | | variable region | | SEQ ID NO: 284 | |
| OC233 | PDGFRβ/VEGF-A | Heavy chain | Cluster # 1044 | US20110177074 | 20462 |
| | | variable region | | SEQ ID NO: 288 | |
| OC234 | PDGFRβ/VEGF-A | Heavy chain | Cluster # 1048 | US20110177074 | 20463 |
| | | variable region | | SEQ ID NO: 292 | |
| OC235 | PDGFRβ/VEGF-A | Heavy chain | Cluster # 1056 | US20110177074 | 20464 |
| | | variable region | | SEQ ID NO: 296 | |
| OC236 | PDGFRβ/VEGF-A | Heavy chain | Cluster # 1064 | US20110177074 | 20465 |
| | | variable region | | SEQ ID NO: 300 | |
| OC237 | PDGFRβ/VEGF-A | Heavy chain | Cluster # 1080 | US20110177074 | 20466 |
| | | variable region | | SEQ ID NO: 304 | |
| OC238 | PDGFRβ/VEGF-A | Heavy chain | Cluster # 1092 | US20110177074 | 20467 |
| | | variable region | | SEQ ID NO: 308 | |
| OC239 | PDGFRβ/VEGF-A | Heavy chain | Cluster # 1094 | US20110177074 | 20468 |
| | | variable region | | SEQ ID NO: 312 | |
| OC240 | PDGFRβ/VEGF-A | Heavy chain | Cluster # 1096 | US20110177074 | 20469 |
| | | variable region | | SEQ ID NO: 316 | |
| OC241 | PDGFRβ/VEGF-A | Heavy chain | Cluster # 947 | US20110177074 | 20470 |
| | | variable region | | SEQ ID NO: 32 | |
| OC242 | PDGFRβ/VEGF-A | Heavy chain | Cluster # 1107 | US20110177074 | 20471 |
| | | variable region | | SEQ ID NO: 320 | |
| OC243 | PDGFRβ/VEGF-A | Heavy chain | Cluster # 1111 | US20110177074 | 20472 |
| | | variable region | | SEQ ID NO: 324 | |
| OC244 | PDGFRβ/VEGF-A | Heavy chain | Cluster # 1123 | US20110177074 | 20473 |
| | | variable region | | SEQ ID NO: 328 | |
| OC245 | PDGFRβ/VEGF-A | Heavy chain | Cluster # 1135 | US20110177074 | 20474 |
| | | variable region | | SEQ ID NO: 332 | |
| OC246 | PDGFRβ/VEGF-A | Heavy chain | Cluster # 1142 | US20110177074 | 20475 |
| | | variable region | | SEQ ID NO: 336 | |
| OC247 | PDGFRβ/VEGF-A | Heavy chain | Cluster # 1155 | US20110177074 | 20476 |
| | | variable region | | SEQ ID NO: 340 | |
| OC248 | PDGFRβ/VEGF-A | Heavy chain | Cluster # 1250 | US20110177074 | 20477 |
| | | variable region | | SEQ ID NO: 344 | |
| OC249 | PDGFRβ/VEGF-A | Heavy chain | Cluster # 1252 | US20110177074 | 20478 |
| | | variable region | | SEQ ID NO: 348 | |
| OC250 | PDGFRβ/VEGF-A | Heavy chain | Cluster # 1254 | US20110177074 | 20479 |
| | | variable region | | SEQ ID NO: 352 | |
| OC251 | PDGFRβ/VEGF-A | Heavy chain | Cluster # 1257 | US20110177074 | 20480 |
| | | variable region | | SEQ ID NO: 356 | |
| OC252 | PDGFRβ/VEGF-A | Heavy chain | Cluster # 949 | US20110177074 | 20481 |
| | | variable region | | SEQ ID NO: 36 | |
| OC253 | PDGFRβ/VEGF-A | Heavy chain | Cluster # 1264 | US20110177074 | 20482 |
| | | variable region | | SEQ ID NO: 360 | |
| OC254 | PDGFRβ/VEGF-A | Heavy chain | Cluster # 1266 | US20110177074 | 20483 |
| | | variable region | | SEQ ID NO: 364 | |
| OC255 | PDGFRβ/VEGF-A | Heavy chain | Cluster # 1268 | US20110177074 | 20484 |
| | | variable region | | SEQ ID NO: 368 | |
| OC256 | PDGFRβ/VEGF-A | Heavy chain | Cluster # 1269 | US20110177074 | 20485 |
| | | variable region | | SEQ ID NO: 372 | |
| OC257 | PDGFRβ/VEGF-A | Heavy chain | Cluster # 1270 | US20110177074 | 20486 |
| | | variable region | | SEQ ID NO: 376 | |
| OC258 | PDGFRβ/VEGF-A | Heavy chain | Cluster # 1281 | US20110177074 | 20487 |
| | | variable region | | SEQ ID NO: 380 | |
| OC259 | PDGFRβ/VEGF-A | Heavy chain | Cluster # 1283 | US20110177074 | 20488 |
| | | variable region | | SEQ ID NO: 384 | |
| OC260 | PDGFRβ/VEGF-A | Heavy chain | Cluster # 1285 | US20110177074 | 20489 |
| | | variable region | | SEQ ID NO: 388 | |
| OC261 | PDGFRβ/VEGF-A | Heavy chain | Cluster # 1409 | US20110177074 | 20490 |
| | | variable region | | SEQ ID NO: 392 | |
| OC262 | PDGFRβ/VEGF-A | Heavy chain | Cluster # 1410 | US20110177074 | 20491 |
| | | variable region | | SEQ ID NO: 396 | |
| OC263 | PDGFRβ/VEGF-A | Heavy chain | Cluster # 975 | US20110177074 | 20492 |
| | | variable region | | SEQ ID NO: 40 | |
| OC264 | PDGFRβ/VEGF-A | Heavy chain | Cluster # 1413 | US20110177074 | 20493 |
| | | variable region | | SEQ ID NO: 400 | |
| OC265 | PDGFRβ/VEGF-A | Heavy chain | Cluster # 1416 | US20110177074 | 20494 |
| | | variable region | | SEQ ID NO: 404 | |
| OC266 | PDGFRβ/VEGF-A | Heavy chain | Cluster # 1420 | US20110177074 | 20495 |
| | | variable region | | SEQ ID NO: 408 | |
| OC267 | PDGFRβ/VEGF-A | Heavy chain | Cluster # 1428 | US20110177074 | 20496 |
| | | variable region | | SEQ ID NO: 412 | |
| OC268 | PDGFRβ/VEGF-A | Heavy chain | Cluster # 1437 | US20110177074 | 20497 |
| | | variable region | | SEQ ID NO: 416 | |
| OC269 | PDGFRβ/VEGF-A | Heavy chain | Cluster # 1449 | US20110177074 | 20498 |
| | | variable region | | SEQ ID NO: 420 | |
| OC270 | PDGFRβ/VEGF-A | Heavy chain | Cluster # 1458 | US20110177074 | 20499 |
| | | variable region | | SEQ ID NO: 424 | |
| OC271 | PDGFRβ/VEGF-A | Heavy chain | Cluster # 1476 | US20110177074 | 20500 |
| | | variable region | | SEQ ID NO: 428 | |
| OC272 | PDGFRβ/VEGF-A | Heavy chain | Cluster # 1479 | US20110177074 | 20501 |
| | | variable region | | SEQ ID NO: 432 | |
| OC273 | PDGFRβ/VEGF-A | Heavy chain | Cluster # 997 | US20110177074 | 20502 |
| | | variable region | | SEQ ID NO: 44 | |
| OC274 | PDGFRβ/VEGF-A | Heavy chain | Cluster # 1035 | US20110177074 | 20503 |
| | | variable region | | SEQ ID NO: 48 | |
| OC275 | PDGFRβ/VEGF-A | Heavy chain | Cluster # 1223 | US20110177074 | 20504 |
| | | variable region | | SEQ ID NO: 52 | |
| OC276 | PDGFRβ/VEGF-A | Heavy chain | Cluster # 1228 | US20110177074 | 20505 |
| | | variable region | | SEQ ID NO: 56 | |
| OC277 | PDGFRβ/VEGF-A | Heavy chain | Cluster # 1230 | US20110177074 | 20506 |
| | | variable region | | SEQ ID NO: 60 | |
| OC278 | PDGFRβ/VEGF-A | Heavy chain | Cluster # 1231 | US20110177074 | 20507 |
| | | variable region | | SEQ ID NO: 64 | |
| OC279 | PDGFRβ/VEGF-A | Heavy chain | Cluster # 1236 | US20110177074 | 20508 |
| | | variable region | | SEQ ID NO: 68 | |
| OC280 | PDGFRβ/VEGF-A | Heavy chain | Cluster # 1238 | US20110177074 | 20509 |
| | | variable region | | SEQ ID NO: 72 | |
| OC281 | PDGFRβ/VEGF-A | Heavy chain | Cluster # 1244 | US20110177074 | 20510 |
| | | variable region | | SEQ ID NO: 76 | |
| OC282 | PDGFRβ/VEGF-A | Heavy chain | Cluster # 1245 | US20110177074 | 20511 |
| | | variable region | | SEQ ID NO: 80 | |
| OC283 | PDGFRβ/VEGF-A | Heavy chain | Cluster # 1299 | US20110177074 | 20512 |
| | | variable region | | SEQ ID NO: 84 | |
| OC284 | PDGFRβ/VEGF-A | Heavy chain | Cluster # 1312 | US20110177074 | 20513 |
| | | variable region | | SEQ ID NO: 88 | |
| OC285 | PDGFRβ/VEGF-A | Heavy chain | Cluster # 1314 | US20110177074 | 20514 |
| | | variable region | | SEQ ID NO: 92 | |
| OC286 | PDGFRβ/VEGF-A | Heavy chain | Cluster # 1317 | US20110177074 | 20515 |
| | | variable region | | SEQ ID NO: 96 | |
| OC287 | PDGFRβ/VEGF-A | Heavy chain | Cluster # 597 | US20110177074 | 20516 |
| | | variable region | | SEQ ID NO: 8 | |
| OC288 | RGMa | Heavy chain | AE12-1 | US20140023659 | 20517 |
| | | variable region | | SEQ ID NO: 1 | |
| OC289 | RGMa | Heavy chain | AE12-20 | US20140023659 | 20518 |
| | | variable region | | SEQ ID NO: 107 | |
| OC290 | RGMa | Heavy chain | AE12-21 | US20140023659 | 20519 |
| | | variable region | | SEQ ID NO: 115 | |
| OC291 | RGMa | Heavy chain | AE12-23 | US20140023659 | 20520 |
| | | variable region | | SEQ ID NO: 123 | |
| OC292 | RGMa | Heavy chain | AE12-24 | US20140023659 | 20521 |
| | | variable region | | SEQ ID NO: 131 | |
| OC293 | RGMa | Heavy chain | AE12-3 | US20140023659 | 20522 |
| | | variable region | | SEQ ID NO: 17 | |
| OC294 | RGMa | Heavy chain | AE12-4 | US20140023659 | 20523 |
| | | variable region | | SEQ ID NO: 25 | |
| OC295 | RGMa | Heavy chain | AE12-5 | US20140023659 | 20524 |
| | | variable region | | SEQ ID NO: 33 | |
| OC296 | RGMa | Heavy chain | AE12-6 | US20140023659 | 20525 |
| | | variable region | | SEQ ID NO: 41 | |
| OC297 | RGMa | Heavy chain | AE12-7 | US20140023659 | 20526 |
| | | variable region | | SEQ ID NO: 49 | |
| OC298 | RGMa | Heavy chain | AE12-8 | US20140023659 | 20527 |
| | | variable region | | SEQ ID NO: 57 | |
| OC299 | RGMa | Heavy chain | AE12-2 | US20140023659 | 20528 |
| | | variable region | | SEQ ID NO: 9 | |
| OC300 | RGMa | Heavy chain | AE12-13 | US20140023659 | 20529 |
| | | variable region | | SEQ ID NO: 91 | |
| OC301 | RGMa | Heavy chain | AE12-15 | US20140023659 | 20530 |
| | | variable region | | SEQ ID NO: 99 | |
| OC302 | S1P4 | Heavy chain | | WO2015057939 | 20531 |
| | | variable region | | SEQ ID NO: 7 | |
| OC303 | VEGF, C5, | Heavy chain | NVS73 | US20140186350 | 20532 |
| | Factor P, Factor | variable region | | SEQ ID 111 | |
| | D, EPO, EPOR, | | | | |
| | IL-1β, IL-17A, | | | | |
| | Il-10, TNFα, or | | | | |
| | FGFR2 | | | | |
| OC304 | VEGF, C5, | Heavy chain | NVS75 | US20140186350 | 20533 |
| | Factor P, Factor | variable region | | SEQ ID 193 | |
| | D, EPO, EPOR, | | | | |
| | IL-1β, IL-17A, | | | | |
| | Il-10, TNFα, or | | | | |
| | FGFR2 | | | | |
| OC305 | VEGF, C5, | Heavy chain | NVS70 | US20140186350 | 20534 |
| | Factor P, Factor | variable region | | SEQ ID 40 | |
| | D, EPO, EPOR, | | | | |
| | IL-1β, IL-17A, | | | | |
| | Il-10, TNFα, or | | | | |
| | FGFR2 | | | | |
| OC306 | VEGF, C5, | Heavy chain | NVS71 | US20140186350 | 20535 |
| | Factor P, Factor | variable region | | SEQ ID 59 | |
| | D, EPO, EPOR, | | | | |
| | IL-1β, IL-17A, | | | | |
| | Il-10, TNFα, or | | | | |
| | FGFR2 | | | | |
| OC307 | VEGF, C5, | Heavy chain | NVS4 | US20140186350 | 20536 |
| | Factor P, Factor | variable region | | SEQ ID 7 | |
| | D, EPO, EPOR, | | | | |
| | IL-1β, IL-17A, | | | | |
| | Il-10, TNFα, or | | | | |
| | FGFR2 | | | | |
| OC308 | VEGF, C5, | Heavy chain | NVS72 | US20140186350 | 20537 |
| | Factor P, Factor | variable region | | SEQ ID 81 | |
| | D, EPO, EPOR, | | | | |
| | IL-1β, IL-17A, | | | | |
| | Il-10, TNFα, or | | | | |
| | FGFR2 | | | | |
| OC309 | VEGF-A | Heavy chain | H6 | US20140086829 | 20538 |
| | | variable region | | SEQ ID 4 | |
| OC310 | VEGF-A | Heavy chain | H5 | US20140086829 | 20539 |
| | | variable region | | SEQ ID 5 | |
| OC311 | VEGF-A | Heavy chain | H7 | US20140086829 | 20540 |
| | | variable region | | SEQ ID 6 | |
| OC312 | C5a | Heavy chain | BNJ364 | US20130224187 | 20541 |
| | | with signal | | SEQ ID 24 | |
| | | peptide | | | |
| OC313 | C5a | Heavy chain | BNJ367, | US20130224187 | 20542 |
| | | with signal | BNJ371, | SEQ ID 32 | |
| | | peptide | BNJ378 | | |
| OC314 | C5a | Heavy chain | BNJ366 | US20130224187 | 20543 |
| | | with signal | | SEQ ID 43 | |
| | | peptide | | | |
| OC315 | C5a | Heavy chain | BNJ369, | US20130224187 | 20544 |
| | | with signal | BNJ381, | SEQ ID 48 | |
| | | peptide | BNJ383 | | |
| OC316 | Annexin IV or a | Light chain | B4 | WO2014116880 | 20545 |
| | phospholipid; | | | SEQ ID 13 | |
| | and (b) a | | | | |
| | complement | | | | |
| | inhibitor | | | | |
| OC317 | Annexin IV or a | Light chain | B4 | WO2014116880 | 20546 |
| | phospholipid; | | | SEQ ID 14 | |
| | and (b) a | | | | |
| | complement | | | | |
| | inhibitor | | | | |
| OC318 | Annexin IV or a | Light chain | C2 | WO2014116880 | 20547 |
| | phospholipid; | | | SEQ ID 34 | |
| | and (b) a | | | | |
| | complement | | | | |
| | inhibitor | | | | |
| OC319 | Annexin IV or a | Light chain | C2 | WO2014116880 | 20548 |
| | phospholipid; | | | SEQ ID 35 | |
| | and (b) a | | | | |
| | complement | | | | |
| | inhibitor | | | | |
| OC320 | C3b | Light chain | rhuMAB 4D5- | U.S. Pat. No. 8,377,437 | 20549 |
| | | | 8 | SEQ ID 13 | |
| OC321 | C3b, Properdin | Light chain | L-1 | WO2015099838 | 20550 |
| | (factor P), | | | SEQ ID 1 | |
| | Factors Ba and | | | | |
| | Bb, C5, C6, C7, | | | | |
| | C8, C9 | | | | |
| OC322 | C3b, Properdin | Light chain | L-10 | WO2015099838 | 20551 |
| | (factor P), | | | SEQ ID 10 | |
| | Factors Ba and | | | | |
| | Bb, C5, C6, C7, | | | | |
| | C8, C9 | | | | |
| OC323 | C3b, Properdin | Light chain | L-11 | WO2015099838 | 20552 |
| | (factor P), | | | SEQ ID 11 | |
| | Factors Ba and | | | | |
| | Bb, C5, C6, C7, | | | | |
| | C8, C9 | | | | |
| OC324 | C3b, Properdin | Light chain | L-12 | WO2015099838 | 20553 |
| | (factor P), | | | SEQ ID 12 | |
| | Factors Ba and | | | | |
| | Bb, C5, C6, C7, | | | | |
| | C8, C9 | | | | |
| OC325 | C3b, Properdin | Light chain | L-13 | WO2015099838 | 20554 |
| | (factor P), | | | SEQ ID 13 | |
| | Factors Ba and | | | | |
| | Bb, C5, C6, C7, | | | | |
| | C8, C9 | | | | |
| OC326 | C3b, Properdin | Light chain | L-14 | WO2015099838 | 20555 |
| | (factor P), | | | SEQ ID 14 | |
| | Factors Ba and | | | | |
| | Bb, C5, C6, C7, | | | | |
| | C8, C9 | | | | |
| OC327 | C3b, Properdin | Light chain | L-15 | WO2015099838 | 20556 |
| | (factor P), | | | SEQ ID 15 | |
| | Factors Ba and | | | | |
| | Bb, C5, C6, C7, | | | | |
| | C8, C9 | | | | |
| OC328 | C3b, Properdin | Light chain | L-16 | WO2015099838 | 20557 |
| | (factor P), | | | SEQ ID 16 | |
| | Factors Ba and | | | | |
| | Bb, C5, C6, C7, | | | | |
| | C8, C9 | | | | |
| OC329 | C3b, Properdin | Light chain | L-17 | WO2015099838 | 20558 |
| | (factor P), | | | SEQ ID 17 | |
| | Factors Ba and | | | | |
| | Bb, C5, C6, C7, | | | | |
| | C8, C9 | | | | |
| OC330 | C3b, Properdin | Light chain | L-18 | WO2015099838 | 20559 |
| | (factor P), | | | SEQ ID 18 | |
| | Factors Ba and | | | | |
| | Bb, C5, C6, C7, | | | | |
| | C8, C9 | | | | |
| OC331 | C3b, Properdin | Light chain | L-19 | WO2015099838 | 20560 |
| | (factor P), | | | SEQ ID 19 | |
| | Factors Ba and | | | | |
| | Bb, C5, C6, C7, | | | | |
| | C8, C9 | | | | |
| OC332 | C3b, Properdin | Light chain | L-2 | WO2015099838 | 20561 |
| | (factor P), | | | SEQ ID 2 | |
| | Factors Ba and | | | | |
| | Bb, C5, C6, C7, | | | | |
| | C8, C9 | | | | |
| OC333 | C3b, Properdin | Light chain | L-20 | WO2015099838 | 20562 |
| | (factor P), | | | SEQ ID 20 | |
| | Factors Ba and | | | | |
| | Bb, C5, C6, C7, | | | | |
| | C8. C9 | | | | |
| OC334 | C3b, Properdin | Light chain | L-21 | WO2015099838 | 20563 |
| | (factor P), | | | SEQ ID 21 | |
| | Factors Ba and | | | | |
| | Bb, C5, C6, C7, | | | | |
| | C8, C9 | | | | |
| OC335 | C3b, Properdin | Light chain | L-22 | WO2015099838 | 20564 |
| | (factor P), | | | SEQ ID 22 | |
| | Factors Ba and | | | | |
| | Bb, C5, C6, C7, | | | | |
| | C8, C9 | | | | |
| OC336 | C3b, Properdin | Light chain | L-23 | WO2015099838 | 20565 |
| | (factor P), | | | SEQ ID 23 | |
| | Factors Ba and | | | | |
| | Bb, C5, C6, C7, | | | | |
| | C8, C9 | | | | |
| OC337 | C3b, Properdin | Light chain | L-24 | WO2015099838 | 20566 |
| | (factor P), | | | SEQ ID 24 | |
| | Factors Ba and | | | | |
| | Bb, C5, C6, C7, | | | | |
| | C8, C9 | | | | |
| OC338 | C3b, Properdin | Light chain | L-25 | WO2015099838 | 20567 |
| | (factor P), | | | SEQ ID NO: 25 | |
| | Factors Ba and | | | | |
| | Bb, C5, C6, C7, | | | | |
| | C8, C9 | | | | |
| OC339 | C3b, Properdin | Light chain | L-26 | WO2015099838 | 20568 |
| | (factor P), | | | SEQ ID NO: 26 | |
| | Factors Ba and | | | | |
| | Bb, C5, C6, C7, | | | | |
| | C8, C9 | | | | |
| OC340 | C3b, Properdin | Light chain | L-27 | WO2015099838 | 20569 |
| | (factor P), | | | SEQ ID NO: 27 | |
| | Factors Ba and | | | | |
| | Bb, C5, C6, C7, | | | | |
| | C8, C9 | | | | |
| OC341 | C3b, Properdin | Light chain | L-28 | WO2015099838 | 20570 |
| | (factor P), | | | SEQ ID NO: 28 | |
| | Factors Ba and | | | | |
| | Bb, C5, C6, C7, | | | | |
| | C8, C9 | | | | |
| OC342 | C3b, Properdin | Light chain | L-29 | WO2015099838 | 20571 |
| | (factor P), | | | SEQ ID NO: 29 | |
| | Factors Ba and | | | | |
| | Bb, C5, C6, C7, | | | | |
| | C8, C9 | | | | |
| OC343 | C3b, Properdin | Light chain | L-3 | WO2015099838 | 20572 |
| | (factor P), | | | SEQ ID NO: 3 | |
| | Factors Ba and | | | | |
| | Bb, C5, C6, C7, | | | | |
| | C8, C9 | | | | |
| OC344 | C3b, Properdin | Light chain | L-30 | WO2015099838 | 20573 |
| | (factor P), | | | SEQ ID NO: 30 | |
| | Factors Ba and | | | | |
| | Bb, C5, C6, C7, | | | | |
| | C8, C9 | | | | |
| OC345 | C3b, Properdin | Light chain | L-31 | WO2015099838 | 20574 |
| | (factor P), | | | SEQ ID NO: 31 | |
| | Factors Ba and | | | | |
| | Bb, C5, C6, C7, | | | | |
| | C8, C9 | | | | |
| OC346 | C3b, Properdin | Light chain | L-32 | WO2015099838 | 20575 |
| | (factor P), | | | SEQ ID NO: 32 | |
| | Factors Ba and | | | | |
| | Bb, C5, C6, C7, | | | | |
| | C8, C9 | | | | |
| OC347 | C3b, Properdin | Light chain | L-33 | WO2015099838 | 20576 |
| | (factor P), | | | SEQ ID NO: 33 | |
| | Factors Ba and | | | | |
| | Bb, C5, C6, C7, | | | | |
| | C8, C9 | | | | |
| OC348 | C3b, Properdin | Light chain | L-34 | WO2015099838 | 20577 |
| | (factor P), | | | SEQ ID NO: 34 | |
| | Factors Ba and | | | | |
| | Bb, C5, C6, C7, | | | | |
| | C8, C9 | | | | |
| OC349 | C3b, Properdin | Light chain | L-35 | WO2015099838 | 20578 |
| | (factor P), | | | SEQ ID NO: 35 | |
| | Factors Ba and | | | | |
| | Bb, C5, C6, C7, | | | | |
| | C8, C9 | | | | |
| OC350 | C3b, Properdin | Light chain | L-36 | WO2015099838 | 20579 |
| | (factor P), | | | SEQ ID NO: 36 | |
| | Factors Ba and | | | | |
| | Bb, C5, C6, C7, | | | | |
| | C8, C9 | | | | |
| OC351 | C3b, Properdin | Light chain | L-37 | WO2015099838 | 20580 |
| | (factor P), | | | SEQ ID NO: 37 | |
| | Factors Ba and | | | | |
| | Bb, C5, C6, C7, | | | | |
| | C8, C9 | | | | |
| OC352 | C3b, Properdin | Light chain | L-38 | WO2015099838 | 20581 |
| | (factor P), | | | SEQ ID NO: 38 | |
| | Factors Ba and | | | | |
| | Bb, C5, C6, C7, | | | | |
| | C8, C9 | | | | |
| OC353 | C3b, Properdin | Light chain | L-39 | WO2015099838 | 20582 |
| | (factor P), | | | SEQ ID NO: 39 | |
| | Factors Ba and | | | | |
| | Bb, C5, C6, C7, | | | | |
| | C8, C9 | | | | |
| OC354 | C3b, Properdin | Light chain | L-4 | WO2015099838 | 20583 |
| | (factor P), | | | SEQ ID NO: 4 | |
| | Factors Ba and | | | | |
| | Bb, C5, C6, C7, | | | | |
| | C8, C9 | | | | |
| OC355 | C3b, Properdin | Light chain | L-40 | WO2015099838 | 20584 |
| | (factor P), | | | SEQ ID NO: 40 | |
| | Factors Ba and | | | | |
| | Bb, C5, C6, C7, | | | | |
| | C8, C9 | | | | |
| OC356 | C3b, Properdin | Light chain | L-41 | WO2015099838 | 20585 |
| | (factor P), | | | SEQ ID NO: 41 | |
| | Factors Ba and | | | | |
| | Bb, C5, C6, C7, | | | | |
| | C8, C9 | | | | |
| OC357 | C3b, Properdin | Light chain | L-42 | WO2015099838 | 20586 |
| | (factor P), | | | SEQ ID NO: 42 | |
| | Factors Ba and | | | | |
| | Bb, C5, C6, C7, | | | | |
| | C8, C9 | | | | |
| OC358 | C3b, Properdin | Light chain | L-43 | WO2015099838 | 20587 |
| | (factor P), | | | SEQ ID NO: 43 | |
| | Factors Ba and | | | | |
| | Bb, C5, C6, C7, | | | | |
| | C8, C9 | | | | |
| OC359 | C3b, Properdin | Light chain | L-5 | WO2015099838 | 20588 |
| | (factor P), | | | SEQ ID NO: 5 | |
| | Factors Ba and | | | | |
| | Bb, C5, C6, C7, | | | | |
| | C8, C9 | | | | |
| OC360 | C3b, Properdin | Light chain | L-6 | WO2015099838 | 20589 |
| | (factor P), | | | SEQ ID NO: 6 | |
| | Factors Ba and | | | | |
| | Bb, C5, C6, C7, | | | | |
| | C8, C9 | | | | |
| OC361 | C3b, Properdin | Light chain | L-7 | WO2015099838 | 20590 |
| | (factor P), | | | SEQ ID NO: 7 | |
| | Factors Ba and | | | | |
| | Bb, C5, C6, C7, | | | | |
| | C8, C9 | | | | |
| OC362 | C3b, Properdin | Light chain | L-8 | WO2015099838 | 20591 |
| | (factor P), | | | SEQ ID NO: 8 | |
| | Factors Ba and | | | | |
| | Bb, C5, C6, C7, | | | | |
| | C8, C9 | | | | |
| OC363 | C3b, Properdin | Light chain | L-9 | WO2015099838 | 20592 |
| | (factor P), | | | SEQ ID NO: 9 | |
| | Factors Ba and | | | | |
| | Bb, C5, C6, C7, | | | | |
| | C8, C9 | | | | |
| OC364 | C5 | Light chain | NVS962 | US20150158936 | 20593 |
| | | | | SEQ ID 10 | |
| OC365 | C5 | Light chain | NVS808 | US20150158936 | 20594 |
| | | | | SEQ ID 108 | |
| OC366 | C5 | Light chain | NVS806 | US20150158936 | 20595 |
| | | | | SEQ ID 122 | |
| OC367 | C5 | Light chain | NVS804 | US20150158936 | 20596 |
| | | | | SEQ ID 136 | |
| OC368 | C5 | Light chain | NVS809 | US20150158936 | 20597 |
| | | | | SEQ ID 150 | |
| OC369 | C5 | Light chain | NVS805 | US20150158936 | 20598 |
| | | | | SEQ ID 164 | |
| OC370 | C5 | Light chain | NVS962-S | US20150158936 | 20599 |
| | | | | SEQ ID 178 | |
| OC371 | C5 | Light chain | NVS962-Q | US20150158936 | 20600 |
| | | | | SEQ ID 192 | |
| OC372 | C5 | Light chain | NVS962-S31A | US20150158936 | 20601 |
| | | | | SEQ ID 206 | |
| OC373 | C5 | Light chain | NVS962-G | US20150158936 | 20602 |
| | | | | SEQ ID 220 | |
| OC374 | C5 | Light chain | NVS962-T | US20150158936 | 20603 |
| | | | | SEQ ID 234 | |
| OC375 | C5 | Light chain | NVS963 | US20150158936 | 20604 |
| | | | | SEQ ID 24 | |
| OC376 | C5 | Light chain | NVS965-T | US20150158936 | 20605 |
| | | | | SEQ ID 248 | |
| OC377 | C5 | Light chain | NVS965-Q | US20150158936 | 20606 |
| | | | | SEQ ID 262 | |
| OC378 | C5 | Light chain | NVS965-S | US20150158936 | 20607 |
| | | | | SEQ ID 276 | |
| OC379 | C5 | Light chain | NVS964 | US20150158936 | 20608 |
| | | | | SEQ ID 38 | |
| OC380 | C5 | Light chain | Antibody 8109 | US20150158936 | 20609 |
| | | | | SEQ ID 419 | |
| OC381 | C5 | Light chain | Antibody 8110 | US20150158936 | 20610 |
| | | | | SEQ ID 435 | |
| OC382 | C5 | Light chain | Antibody 8111 | US20150158936 | 20611 |
| | | | | SEQ ID 450 | |
| OC383 | C5 | Light chain | Antibody 8113 | US20150158936 | 20612 |
| | | | | SEQ ID 463 | |
| OC384 | C5 | Light chain | Antibody 8114 | US20150158936 | 20613 |
| | | | | SEQ ID 479 | |
| OC385 | C5 | Light chain | NVS966 | US20150158936 | 20614 |
| | | | | SEQ ID 52 | |
| OC386 | C5 | Light chain | NVS965 | US20150158936 | 20615 |
| | | | | SEQ ID 66 | |
| OC387 | C5 | Light chain | NVS967 | US20150158936 | 20616 |
| | | | | SEQ ID 80 | |
| OC388 | C5 | Light chain | NVS807 | US20150158936 | 20617 |
| | | | | SEQ ID 94 | |
| OC389 | C5 | Light chain | L1 | US20150239966 | 20618 |
| | | | | SEQ ID 1 | |
| OC390 | C5 | Light chain | L6 | US20150239966 | 20619 |
| | | | | SEQ ID NO: 11 | |
| OC391 | C5 | Light chain | L2 | US20150239966 | 20620 |
| | | | | SEQ ID 3 | |
| OC392 | C5 | Light chain | L3 | US20150239966 | 20621 |
| | | | | SEQ ID NO: 5 | |
| OC393 | C5 | Light chain | L4 | US20150239966 | 20622 |
| | | | | SEQ ID NO: 7 | |
| OC394 | C5 | Light chain | L5 | US20150239966 | 20623 |
| | | | | SEQ ID NO: 9 | |
| OC395 | C5 | Light chain | Tesidolumab, | U.S. Pat. No. 8,241,628 | 20624 |
| | | | “LFG 316, | SEQ ID 10 | |
| | | | LFG-316, | | |
| | | | LFG316” | | |
| OC396 | C5 | Light chain | | U.S. Pat. No. 9,133,269 | 20625 |
| | | | | SEQ ID 10 | |
| OC397 | C5 | Light chain | | U.S. Pat. No. 9,133,269 | 20626 |
| | | | | SEQ ID 11 | |
| OC398 | C5 | Light chain | | U.S. Pat. No. 9,133,269 | 20627 |
| | | | | SEQ ID 12 | |
| OC399 | C5 | Light chain | | U.S. Pat. No. 9,133,269 | 20628 |
| | | | | SEQ ID 13 | |
| OC400 | C5 | Light chain | | U.S. Pat. No. 9,133,269 | 20629 |
| | | | | SEQ ID 14 | |
| OC401 | C5 | Light chain | | U.S. Pat. No. 9,133,269 | 20630 |
| | | | | SEQ ID 15 | |
| OC402 | C5 | Light chain | | U.S. Pat. No. 9,133,269 | 20631 |
| | | | | SEQ ID 16 | |
| OC403 | C5 | Light chain | | U.S. Pat. No. 9,133,269 | 20632 |
| | | | | SEQ ID 17 | |
| OC404 | C5 | Light chain | | U.S. Pat. No. 9,133,269 | 20633 |
| | | | | SEQ ID 18 | |
| OC405 | C5 | Light chain | | U.S. Pat. No. 9,133,269 | 20634 |
| | | | | SEQ ID 19 | |
| OC406 | C5 | Light chain | | U.S. Pat. No. 9,133,269 | 20635 |
| | | | | SEQ ID 20 | |
| OC407 | C5 | Light chain | | U.S. Pat. No. 9,133,269 | 20636 |
| | | | | SEQ ID 21 | |
| OC408 | C5 | Light chain | | U.S. Pat. No. 9,133,269 | 20637 |
| | | | | SEQ ID 22 | |
| OC409 | C5 | Light chain | | U.S. Pat. No. 9,133,269 | 20638 |
| | | | | SEQ ID 23 | |
| OC410 | C5 | Light chain | | U.S. Pat. No. 9,133,269 | 20639 |
| | | | | SEQ ID 24 | |
| OC411 | C5 | Light chain | | U.S. Pat. No. 9,133,269 | 20640 |
| | | | | SEQ ID 6 | |
| OC412 | C5 | Light chain | | U.S. Pat. No. 9,133,269 | 20641 |
| | | | | SEQ ID 7 | |
| OC413 | C5 | Light chain | | U.S. Pat. No. 9,133,269 | 20642 |
| | | | | SEQ ID 8 | |
| OC414 | C5 | Light chain | | U.S. Pat. No. 9,133,269 | 20643 |
| | | | | SEQ ID 9 | |
| OC415 | C5a | Light chain | BNJ364, | US20130224187 | 20644 |
| | | | BNJ367, | SEQ ID 17 | |
| | | | BNJ366, | | |
| | | | BNJ369 | | |
| OC416 | C5a | Light chain | BNJ371, | US20130224187 | 20645 |
| | | | BNJ381 | SEQ ID 36 | |
| OC417 | C5a | Light chain | BNJ378, | US20130224187 | 20646 |
| | | | BNJ383 | SEQ ID 40 | |
| OC418 | CGRP | Light chain | Ab11 | US20120294802 | 20647 |
| | | | | SEQ ID NO: 102 | |
| OC419 | CGRP | Light chain | Ab12 | US20120294802 | 20648 |
| | | | | SEQ ID NO: 112 | |
| OC420 | CGRP | Light chain | Ab2 | US20120294802 | 20649 |
| | | | | SEQ ID NO: 12 | |
| OC421 | CGRP | Light chain | Ab13 | US20120294802 | 20650 |
| | | | | SEQ ID NO: 122 | |
| OC422 | CGRP | Light chain | Ab14 | US20120294802 | 20651 |
| | | | | SEQ ID NO: 132 | |
| OC423 | CGRP | Light chain | Ab1 | US20120294802 | 20652 |
| | | | | SEQ ID NO: 2 | |
| OC424 | CGRP | Light chain | Ab3 | US20120294802 | 20653 |
| | | | | SEQ ID NO: 22 | |
| OC425 | CGRP | Light chain | Ab4 | US20120294802 | 20654 |
| | | | | SEQ ID NO: 32 | |
| OC426 | CGRP | Light chain | Ab5 | US20120294802 | 20655 |
| | | | | SEQ ID NO: 42 | |
| OC427 | CGRP | Light chain | Ab6 | US20120294802 | 20656 |
| | | | | SEQ ID NO: 52 | |
| OC428 | CGRP | Light chain | Ab7 | US20120294802 | 20657 |
| | | | | SEQ ID NO: 62 | |
| OC429 | CGRP | Light chain | Ab8 | US20120294802 | 20658 |
| | | | | SEQ ID NO: 72 | |
| OC430 | CGRP | Light chain | Ab9 | US20120294802 | 20659 |
| | | | | SEQ ID NO: 82 | |
| OC431 | CGRP | Light chain | Ab10 | US20120294802 | 20660 |
| | | | | SEQ ID NO: 92 | |
| OC432 | Factor D | Light chain | Fab 238 | WO2009134711 | 20661 |
| | | | | SEQ ID NO: 47 | |
| OC433 | Factor D, | Light Chain | Lampalizumab, | U.S. Pat. No. 8,273,352 | 20662 |
| | humanized | | | SEQ ID NO: 47 | |
| | IgG2 | | | | |
| OC434 | platelet-derived | Light chain | Rinucumab, | | 20663 |
| | growth factor | | REGN2176 | | |
| | receptor beta | | | | |
| | PDGFRB | | | | |
| OC435 | S1P4 | Light chain | | WO2015057939 | 20664 |
| | | | | SEQ ID NO: 41 | |
| OC436 | VEGF, C5, | Light chain | NVS73, | US20140186350 | 20665 |
| | Factor P, Factor | | | SEQ ID 122 | |
| | D, EPO, EPOR, | | | | |
| | IL-1β, IL-17A, | | | | |
| | Il-10, INFα, or | | | | |
| | FGFR2 | | | | |
| OC437 | VEGF, C5, | Light chain | NVS81 | US20140186350 | 20666 |
| | Factor P, Factor | | | SEQ ID 158 | |
| | D, EPO, EPOR, | | | | |
| | IL-1β, IL-17A, | | | | |
| | Il-10, INFα, or | | | | |
| | FGFR2 | | | | |
| OC438 | VEGF, C5, | Light chain | NVS81T | US20140186350 | 20667 |
| | Factor P, Factor | | | SEQ ID 160 | |
| | D, EPO, EPOR, | | | | |
| | IL-1β, IL-17A, | | | | |
| | Il-10, TNFα, or | | | | |
| | FGFR2 | | | | |
| OC439 | VEGF, C5, | Light chain | NVS82 | US20140186350 | 20668 |
| | Factor P, Factor | | | SEQ ID 162 | |
| | D, EPO, EPOR, | | | | |
| | IL-1β, IL-17A, | | | | |
| | Il-10, TNFα, or | | | | |
| | FGFR2 | | | | |
| OC440 | VEGF, C5, | Light chain | NVS82T | US20140186350 | 20669 |
| | Factor P, Factor | | | SEQ ID 164 | |
| | D, EPO, EPOR, | | | | |
| | IL-1β, IL-17A, | | | | |
| | Il-10, TNFα, or | | | | |
| | FGFR2 | | | | |
| OC441 | VEGF, C5, | Light chain | NVS1b | US20140186350 | 20670 |
| | Factor P, Factor | | | SEQ ID 172 | |
| | D, EPO, EPOR, | | | | |
| | IL-1β, IL-17A, | | | | |
| | Il-10, TNFα, or | | | | |
| | FGFR2 | | | | |
| OC442 | VEGF, C5, | Light chain | NVS1c | US20140186350 | 20671 |
| | Factor P, Factor | | | SEQ ID 174 | |
| | D, EPO, EPOR, | | | | |
| | IL-1β, IL-17A, | | | | |
| | Il-10, TNFα, or | | | | |
| | FGFR2 | | | | |
| OC443 | VEGF, C5, | Light chain | NVS1d | US20140186350 | 20672 |
| | Factor P, Factor | | | SEQ ID 176 | |
| | D, EPO, EPOR, | | | | |
| | IL-1β, IL-17A, | | | | |
| | Il-10, TNFα, or | | | | |
| | FGFR2 | | | | |
| OC444 | VEGF, C5, | Light chain | NVS1e | US20140186350 | 20673 |
| | Factor P, Factor | | | SEQ ID 178 | |
| | D, EPO, EPOR, | | | | |
| | IL-1β, IL-17A, | | | | |
| | Il-10, TNFα, or | | | | |
| | FGFR2 | | | | |
| OC445 | VEGF, C5, | Light chain | NVS1f | US20140186350 | 20674 |
| | Factor P, Factor | | | SEQ ID 180 | |
| | D, EPO, EPOR, | | | | |
| | IL-1β, IL-17A, | | | | |
| | Il-10, TNFα, or | | | | |
| | FGFR2 | | | | |
| OC446 | VEGF, C5, | Light chain | NVS1g | US20140186350 | 20675 |
| | Factor P, Factor | | | SEQ ID 182 | |
| | D, EPO, EPOR, | | | | |
| | IL-1β, IL-17A, | | | | |
| | Il-10, TNFα, or | | | | |
| | FGFR2 | | | | |
| OC447 | VEGF, C5, | Light chain | NVS1h | US20140186350 | 20676 |
| | Factor P, Factor | | | SEQ ID 184 | |
| | D, EPO, EPOR, | | | | |
| | IL-1β, IL-17A, | | | | |
| | Il-10, TNFα, or | | | | |
| | FGFR2 | | | | |
| OC448 | VEGF, C5, | Light chain | NVS1j | US20140186350 | 20677 |
| | Factor P, Factor | | | SEQ ID 185 | |
| | D, EPO, EPOR, | | | | |
| | IL-1β, IL-17A, | | | | |
| | Il-10, TNFα, or | | | | |
| | FGFR2 | | | | |
| OC449 | VEGF, C5, | Light chain | NVS4, NVS1, | US20140186350 | 20678 |
| | Factor P, Factor | | NVS2, NVS3, | SEQ ID 19 | |
| | D, EPO, EPOR, | | NVS36, | | |
| | IL-1β, IL-17A, | | NVS37 | | |
| | Il-10, TNFα, or | | | | |
| | FGFR2 | | | | |
| OC450 | VEGF, C5, | Light chain | NVS75, | US20140186350 | 20679 |
| | Factor P, Factor | | NVS74T, | SEQ ID 202 | |
| | D, EPO, EPOR, | | NCS75T | | |
| | IL-1β, IL-17A, | | | | |
| | Il-10, TNFα, or | | | | |
| | FGFR2 | | | | |
| OC451 | VEGF, C5, | Light chain | NVS70, | US20140186350 | 20680 |
| | Factor P, Factor | | NVS70T | SEQ ID 51 | |
| | D, EPO, EPOR, | | | | |
| | IL-1β, IL-17A, | | | | |
| | Il-10, TNFα, or | | | | |
| | FGFR2 | | | | |
| OC452 | VEGF, C5, | Light chain | NVS71, | US20140186350 | 20681 |
| | Factor P, Factor | | NVS71T | SEQ ID 73 | |
| | D, EPO, EPOR, | | | | |
| | IL-1β, IL-17A, | | | | |
| | Il-10, TNFα, or | | | | |
| | FGFR2 | | | | |
| OC453 | VEGF, C5, | Light chain | NVS72, | US20140186350 | 20682 |
| | Factor P, Factor | | NVS72T | SEQ ID 95 | |
| | D, EPO, EPOR, | | | | |
| | IL-1β, IL-17A, | | | | |
| | Il-10, TNFα, or | | | | |
| | FGFR2 | | | | |
| OC454 | sphingosine-1- | Light chain | Sonepcizumab, | | 20683 |
| | phosphate | full | S1P-LT1012 | | |
| OC455 | sphingosine-1- | Light chain | Sonepcizumab, | | 20684 |
| | phosphate | variable | S1P-LT1010 | | |
| OC456 | Factor D | Light chain | Humanized | WO2009134711 | 20685 |
| | | variable region | Clone #111 | SEQ ID NO: 1 | |
| OC457 | Factor D | Light chain | Fab 238-4 | WO2009134711 | 20686 |
| | | variable region | | SEQ ID NO: 10 | |
| OC458 | Factor D | Light chain | Fab 238-5 | WO2009134711 | 20687 |
| | | variable region | | SEQ ID NO: 11 | |
| OC459 | Factor D | Light chain | Fab 238-6 | WO2009134711 | 20688 |
| | | variable region | | SEQ ID NO: 12 | |
| OC460 | Factor D | Light chain | Fab 238-7 | WO2009134711 | 20689 |
| | | variable region | | SEQ ID NO: 13 | |
| OC461 | Factor D | Light chain | Fab 238-8 | WO2009134711 | 20690 |
| | | variable region | | SEQ ID NO: 14 | |
| OC462 | Factor D | Light chain | Fab 238-9 | WO2009134711 | 20691 |
| | | variable region | | SEQ ID NO: 15 | |
| OC463 | Factor D | Light chain | Fab 238-10 | WO2009134711 | 20692 |
| | | variable region | | SEQ ID NO: 16 | |
| OC464 | Factor D | Light chain | Fab 238-11 | WO2009134711 | 20693 |
| | | variable region | | SEQ ID NO: 17 | |
| OC465 | Factor D | Light chain | L243 | WO2009134711 | 20694 |
| | | variable region | | SEQ ID NO: 32 | |
| OC466 | Factor D | Light chain | humanized | WO2009134711 | 20695 |
| | | variable region | L243 | SEQ ID NO: 36 | |
| OC467 | Factor D | Light chain | Fab 238 | WO2009134711 | 20696 |
| | | variable region | | SEQ ID NO: 6 | |
| OC468 | Factor D | Light chain | Fab 238-1 | WO2009134711 | 20697 |
| | | variable region | | SEQ ID NO: 7 | |
| OC469 | Factor D | Light chain | Fab 238-2 | WO2009134711 | 20698 |
| | | variable region | | SEQ ID NO: 8 | |
| OC470 | Factor D | Light chain | Fab 238-3 | WO2009134711 | 20699 |
| | | variable region | | SEQ ID NO: 9 | |
| OC471 | LPG | Light chain | #7 | U.S. Pat. No. 8,591,902 | 20700 |
| | (lysophosphati- | variable region | | SEQ ID NO: 17 | |
| | dylglucoside) | | | | |
| OC472 | LPG | Light chain | #15 | U.S. Pat. No. 8,591,902 | 20701 |
| | (lysophosphati- | variable region | | SEQ ID NO: 7 | |
| | dylglucoside) | | | | |
| OC473 | PDGFR-beta | Light chain | 3299N | US20140193402 | 20702 |
| | | variable region | | SEQ ID 10 | |
| OC474 | PDGFR-beta | Light chain | 3368N | US20140193402 | 20703 |
| | | variable region | | SEQ ID 106 | |
| OC475 | PDGFR-beta | Light chain | 3373N | US20140193402 | 20704 |
| | | variable region | | SEQ ID 122 | |
| OC476 | PDGFR-beta | Light chain | 3374N | US20140193402 | 20705 |
| | | variable region | | SEQ ID 138 | |
| OC477 | PDGFR-beta | Light chain | 3094P | US20140191402 | 20706 |
| | | variable region | | SEQ ID 154 | |
| OC478 | PDGFR-beta | Light chain | 3095S | US20140193402 | 20707 |
| | | variable region | | SEQ ID 170 | |
| OC479 | PDGFR-beta | Light chain | 3096S | US20140193402 | 20708 |
| | | variable region | | SEQ ID 186 | |
| OC480 | PDGFR-beta | Light chain | 3097S | US20140193402 | 20709 |
| | | variable region | | SEQ ID 202 | |
| OC481 | PDGFR-beta | Light chain | 3098S | US20140193402 | 20710 |
| | | variable region | | SEQ ID 218 | |
| OC482 | PDGFR-beta | Light chain | 3099S | US20140193402 | 20711 |
| | | variable region | | SEQ ID 234 | |
| OC483 | PDGFR-beta | Light chain | 3102S | US20140193402 | 20712 |
| | | variable region | | SEQ ID 250 | |
| OC484 | PDGFR-beta | Light chain | 3305N | US20140193402 | 20713 |
| | | variable region | | SEQ ID 26 | |
| OC485 | PDGFR-beta | Light chain | 3103S | US20140193402 | 20714 |
| | | variable region | | SEQ ID 266 | |
| OC486 | PDGFR-beta | Light chain | 3104S | US20140193402 | 20715 |
| | | variable region | | SEQ ID 282 | |
| OC487 | PDGFR-beta | Light chain | 3105S | US20140193402 | 20716 |
| | | variable region | | SEQ ID 298 | |
| OC488 | PDGFR-beta | Light chain | 3106S | US20140193402 | 20717 |
| | | variable region | | SEQ ID 314 | |
| OC489 | PDGFR-beta | Light chain | 3107S | US20140193402 | 20718 |
| | | variable region | | SEQ ID 330 | |
| OC490 | PDGFR-beta | Light chain | 3310N | US20140193402 | 20719 |
| | | variable region | | SEQ ID 42 | |
| OC491 | PDGFR-beta | Light chain | 3361N | US20140193402 | 20720 |
| | | variable region | | SEQ ID 58 | |
| OC492 | PDGFR-beta | Light chain | 3363N | US20140193402 | 20721 |
| | | variable region | | SEQ ID 74 | |
| OC493 | PDGFR-beta | Light chain | 3365N | US20140193402 | 20722 |
| | | variable region | | SEQ ID 90 | |
| OC494 | PDGFRβ/VEGF-A | Light chain | Cluster # 600 | US20110177074 | 20723 |
| | | variable region | | SEQ ID NO: 10 | |
| OC495 | PDGFRβ/VEGF-A | Light chain | Cluster # 1323 | US20110177074 | 20724 |
| | | variable region | | SEQ ID NO: 102 | |
| OC496 | PDGFRβ/VEGF-A | Light chain | Cluster # 1330 | US20110177074 | 20725 |
| | | variable region | | SEQ ID NO: 106 | |
| OC497 | PDGFRβ/VEGF-A | Light chain | Cluster # 1334 | US20110177074 | 20726 |
| | | variable region | | SEQ ID NO: 110 | |
| OC498 | PDGFRβ/VEGF-A | Light chain | Cluster # 1345 | US20110177074 | 20727 |
| | | variable region | | SEQ ID NO: 114 | |
| OC499 | PDGFRβ/VEGF-A | Light chain | Cluster # 1346 | US20110177074 | 20728 |
| | | variable region | | SEQ ID NO: 118 | |
| OC500 | PDGFRβ/VEGF-A | Light chain | Cluster # 1359 | US20110177074 | 20729 |
| | | variable region | | SEQ ID NO: 122 | |
| OC501 | PDGFRβ/VEGF-A | Light chain | Cluster # 1365 | US20110177074 | 20730 |
| | | variable region | | SEQ ID NO: 126 | |
| OC502 | PDGFRβ/VEGF-A | Light chain | Cluster # 1402 | US20110177074 | 20731 |
| | | variable region | | SEQ ID NO: 130 | |
| OC503 | PDGFRβ/VEGF-A | Light chain | Cluster # 1515 | US20110177074 | 20732 |
| | | variable region | | SEQ ID NO: 134 | |
| OC504 | PDGFRβ/VEGF-A | Light chain | Cluster # 1531 | US20110177074 | 20733 |
| | | variable region | | SEQ ID NO: 138 | |
| OC505 | PDGFRβ/VEGF-A | Light chain | Cluster # 607 | US20110177074 | 20734 |
| | | variable region | | SEQ ID NO: 14 | |
| OC506 | PDGFRβ/VEGF-A | Light chain | Cluster # 1535 | US20110177074 | 20735 |
| | | variable region | | SEQ ID NO: 142 | |
| OC507 | PDGFRβ/VEGF-A | Light chain | Cluster # 1541 | US20110177074 | 20736 |
| | | variable region | | SEQ ID NO: 146 | |
| OC508 | PDGFRβ/VEGF-A | Light chain | Cluster # 1550 | US20110177074 | 20737 |
| | | variable region | | SEQ ID NO: 150 | |
| OC509 | PDGFRβ/VEGF-A | Light chain | Cluster # 1564 | US20110177074 | 20738 |
| | | variable region | | SEQ ID NO: 154 | |
| OC510 | PDGFRβ/VEGF-A | Light chain | Cluster # 1601 | US20110177074 | 20739 |
| | | variable region | | SEQ ID NO: 158 | |
| OC511 | PDGFRβ/VEGF-A | Light chain | Cluster # 1629 | US20110177074 | 20740 |
| | | variable region | | SEQ ID NO: 162 | |
| OC512 | PDGFRβ/VEGF-A | Light chain | Cluster # 635 | US20110177074 | 20741 |
| | | variable region | | SEQ ID NO: 166 | |
| OC513 | PDGFRβ/VEGF-A | Light chain | Cluster # 636 | US20110177074 | 20742 |
| | | variable region | | SEQ ID NO: 170 | |
| OC514 | PDGFRβ/VEGF-A | Light chain | Cluster # 638 | US20110177074 | 20743 |
| | | variable region | | SEQ ID NO: 174 | |
| OC515 | PDGFRβ/VEGF-A | Light chain | Cluster # 656 | US20110177074 | 20744 |
| | | variable region | | SEQ ID NO: 178 | |
| OC516 | PDGFRβ/VEGF-A | Light chain | Cluster # 613 | US20110177074 | 20745 |
| | | variable region | | SEQ ID NO: 18 | |
| OC517 | PDGFRβ/VEGF-A | Light chain | Cluster # 665 | US20110177074 | 20746 |
| | | variable region | | SEQ ID NO: 182 | |
| OC518 | PDGFRβ/VEGF-A | Light chain | Cluster # 668 | US20110177074 | 20747 |
| | | variable region | | SEQ ID NO: 186 | |
| OC519 | PDGFRβ/VEGF-A | Light chain | Cluster # 669 | US20110177074 | 20748 |
| | | variable region | | SEQ ID NO: 190 | |
| OC520 | PDGFRβ/VEGF-A | Light chain | Cluster # 679 | US20110177074 | 20749 |
| | | variable region | | SEQ ID NO: 194 | |
| OC521 | PDGFRβ/VEGF-A | Light chain | Cluster # 695 | US20110177074 | 20750 |
| | | variable region | | SEQ ID NO: 198 | |
| OC522 | PDGFRβ/VEGF-A | Light chain | Cluster # 709 | US20110177074 | 20751 |
| | | variable region | | SEQ ID NO: 202 | |
| OC523 | PDGFRβ/VEGF-A | Light chain | Cluster # 710 | US20110177074 | 20752 |
| | | variable region | | SEQ ID NO: 206 | |
| OC524 | PDGFRβ/VEGF-A | Light chain | Cluster # 741 | US20110177074 | 20753 |
| | | variable region | | SEQ ID NO: 210 | |
| OC525 | PDGFRβ/VEGF-A | Light chain | Cluster # 752 | US20110177074 | 20754 |
| | | variable region | | SEQ ID NO: 214 | |
| OC526 | PDGFRβ/VEGF-A | Light chain | Cluster # 772 | US20110177074 | 20755 |
| | | variable region | | SEQ ID NO: 218 | |
| OC527 | PDGFRβ/VEGF-A | Light chain | Cluster # 941 | US20110177074 | 20756 |
| | | variable region | | SEQ ID NO: 22 | |
| OC528 | PDGFRβ/VEGF-A | Light chain | Cluster # 779 | US20110177074 | 20757 |
| | | variable region | | SEQ ID NO: 222 | |
| OC529 | PDGFRβ/VEGF-A | Light chain | Cluster # 799 | US20110177074 | 20758 |
| | | variable region | | SEQ ID NO: 226 | |
| OC530 | PDGFRβ/VEGF-A | Light chain | Cluster # 830 | US20110177074 | 20759 |
| | | variable region | | SEQ ID NO: 230 | |
| OC531 | PDGFRβ/VEGF-A | Light chain | Cluster # 844 | US20110177074 | 20760 |
| | | variable region | | SEQ ID NO: 234 | |
| OC532 | PDGFRβ/VEGF-A | Light chain | Cluster # 847 | US20110177074 | 20761 |
| | | variable region | | SEQ ID NO: 238 | |
| OC533 | PDGFRβ/VEGF-A | Light chain | Cluster # 868 | US20110177074 | 20762 |
| | | variable region | | SEQ ID NO: 242 | |
| OC534 | PDGFRβ/VEGF-A | Light chain | Cluster # 870 | US20110177074 | 20763 |
| | | variable region | | SEQ ID NO: 246 | |
| OC535 | PDGFRβ/VEGF-A | Light chain | Cluster # 883 | US20110177074 | 20764 |
| | | variable region | | SEQ ID NO: 250 | |
| OC536 | PDGFRβ/VEGF-A | Light chain | Cluster # 887 | US20110177074 | 20765 |
| | | variable region | | SEQ ID NO: 254 | |
| OC537 | PDGFRβ/VEGF-A | Light chain | Cluster # 901 | US20110177074 | 20766 |
| | | variable region | | SEQ ID NO: 258 | |
| OC538 | PDGFRβ/VEGF-A | Light chain | Cluster # 946 | US20110177074 | 20767 |
| | | variable region | | SEQ ID NO: 26 | |
| OC539 | PDGFRβ/VEGF-A | Light chain | Cluster # 905 | US20110177074 | 20768 |
| | | variable region | | SEQ ID NO: 262 | |
| OC540 | PDGFRβ/VEGF-A | Light chain | Cluster # 909 | US20110177074 | 20769 |
| | | variable region | | SEQ ID NO: 266 | |
| OC541 | PDGFRβ/VEGF-A | Light chain | Cluster # 928 | US20110177074 | 20770 |
| | | variable region | | SEQ ID NO: 270 | |
| OC542 | PDGFRβ/VEGF-A | Light chain | Cluster # 1036 | US20110177074 | 20771 |
| | | variable region | | SEQ ID NO: 274 | |
| OC543 | PDGFRβ/VEGF-A | Light chain | Cluster # 1039 | US20110177074 | 20772 |
| | | variable region | | SEQ ID NO: 278 | |
| OC544 | PDGFRβ/VEGF-A | Light chain | Cluster # 1040 | US20110177074 | 20773 |
| | | variable region | | SEQ ID NO: 282 | |
| OC545 | PDGFRβ/VEGF-A | Light chain | Cluster # 1044 | US20110177074 | 20774 |
| | | variable region | | SEQ ID NO: 286 | |
| OC546 | PDGFRβ/VEGF-A | Light chain | Cluster # 1048 | US20110177074 | 20775 |
| | | variable region | | SEQ ID NO: 290 | |
| OC547 | PDGFRβ/VEGF-A | Light chain | Cluster # 1056 | US20110177074 | 20776 |
| | | variable region | | SEQ ID NO: 294 | |
| OC548 | PDGFRβ/VEGF-A | Light chain | Cluster # 1064 | US20110177074 | 20777 |
| | | variable region | | SEQ ID NO: 298 | |
| OC549 | PDGFRβ/VEGF-A | Light chain | Cluster # 947 | US20110177074 | 20778 |
| | | variable region | | SEQ ID NO: 30 | |
| OC550 | PDGFRβ/VEGF-A | Light chain | Cluster # 1080 | US20110177074 | 20779 |
| | | variable region | | SEQ ID NO: 302 | |
| OC551 | PDGFRβ/VEGF-A | Light chain | Cluster # 1092 | US20110177074 | 20780 |
| | | variable region | | SEQ ID NO: 306 | |
| OC552 | PDGFRβ/VEGF-A | Light chain | Cluster # 1094 | US20110177074 | 20781 |
| | | variable region | | SEQ ID NO: 310 | |
| OC553 | PDGFRβ/VEGF-A | Light chain | Cluster # 1096 | US20110177074 | 20782 |
| | | variable region | | SEQ ID NO: 314 | |
| OC554 | PDGFRβ/VEGF-A | Light chain | Cluster # 1107 | US20110177074 | 20783 |
| | | variable region | | SEQ ID NO: 318 | |
| OC555 | PDGFRβ/VEGF-A | Light chain | Cluster # 1111 | US20110177074 | 20784 |
| | | variable region | | SEQ ID NO: 322 | |
| OC556 | PDGFRβ/VEGF-A | Light chain | Cluster # 1123 | US20110177074 | 20785 |
| | | variable region | | SEQ ID NO: 326 | |
| OC557 | PDGFRβ/VEGF-A | Light chain | Cluster # 1135 | US20110177074 | 20786 |
| | | variable region | | SEQ ID NO: 330 | |
| OC558 | PDGFRβ/VEGF-A | Light chain | Cluster # 1142 | US20110177074 | 20787 |
| | | variable region | | SEQ ID NO: 334 | |
| OC559 | PDGFRβ/VEGF-A | Light chain | Cluster # 1155 | US20110177074 | 20788 |
| | | variable region | | SEQ ID NO: 338 | |
| OC560 | PDGFRβ/VEGF-A | Light chain | Cluster # 949 | US20110177074 | 20789 |
| | | variable region | | SEQ ID NO: 34 | |
| OC561 | PDGFRβ/VEGF-A | Light chain | Cluster # 1250 | US20110177074 | 20790 |
| | | variable region | | SEQ ID NO: 342 | |
| OC562 | PDGFRβ/VEGF-A | Light chain | Cluster # 1252 | US20110177074 | 20791 |
| | | variable region | | SEQ ID NO: 346 | |
| OC563 | PDGFRβ/VEGF-A | Light chain | Cluster # 1254 | US20110177074 | 20792 |
| | | variable region | | SEQ ID NO: 350 | |
| OC564 | PDGFRβ/VEGF-A | Light chain | Cluster # 1257 | US20110177074 | 20793 |
| | | variable region | | SEQ ID NO: 354 | |
| OC565 | PDGFRβ/VEGF-A | Light chain | Cluster # 1264 | US20110177074 | 20794 |
| | | variable region | | SEQ ID NO: 358 | |
| OC566 | PDGFRβ/VEGF-A | Light chain | Cluster # 1266 | US20110177074 | 20795 |
| | | variable region | | SEQ ID NO: 362 | |
| OC567 | PDGFRβ/VEGF-A | Light chain | Cluster # 1268 | US20110177074 | 20796 |
| | | variable region | | SEQ ID NO: 366 | |
| OC568 | PDGFRβ/VEGF-A | Light chain | Cluster # 1269 | US20110177074 | 20797 |
| | | variable region | | SEQ ID NO: 370 | |
| OC569 | PDGFRβ/VEGF-A | Light chain | Cluster #1270 | US20110177074 | 20798 |
| | | variable region | | SEQ ID NO: 374 | |
| OC570 | PDGFRβ/VEGF-A | Light chain | Cluster # 1281 | US20110177074 | 20799 |
| | | variable region | | SEQ ID NO: 378 | |
| OC571 | PDGFRβ/VEGF-A | Light chain | Cluster # 975 | US20110177074 | 20800 |
| | | variable region | | SEQ ID NO: 38 | |
| OC572 | PDGFRβ/VEGF-A | Light chain | Cluster # 1283 | US20110177074 | 20801 |
| | | variable region | | SEQ ID NO: 382 | |
| OC573 | PDGFRβ/VEGF-A | Light chain | Cluster # 1285 | US20110177074 | 20802 |
| | | variable region | | SEQ ID NO: 386 | |
| OC574 | PDGFRβ/VEGF-A | Light chain | Cluster # 1409 | US20110177074 | 20803 |
| | | variable region | | SEQ ID NO: 390 | |
| OC575 | PDGFRβ/VEGF-A | Light chain | Cluster # 1410 | US20110177074 | 20804 |
| | | variable region | | SEQ ID NO: 394 | |
| OC576 | PDGFRβ/VEGF-A | Light chain | Cluster # 1413 | US20110177074 | 20805 |
| | | variable region | | SEQ ID NO: 398 | |
| OC577 | PDGFRβ/VEGF-A | Light chain | Cluster # 1416 | US20110177074 | 20806 |
| | | variable region | | SEQ ID NO: 402 | |
| OC578 | PDGFRβ/VEGF-A | Light chain | Cluster # 1420 | US20110177074 | 20807 |
| | | variable region | | SEQ ID NO: 406 | |
| OC579 | PDGFRβ/VEGF-A | Light chain | Cluster # 1428 | US20110177074 | 20808 |
| | | variable region | | SEQ ID NO: 410 | |
| OC580 | PDGFRβ/VEGF-A | Light chain | Cluster # 1437 | US20110177074 | 20809 |
| | | variable region | | SEQ ID NO: 414 | |
| OC581 | PDGFRβ/VEGF-A | Light chain | Cluster # 1449 | US20110177074 | 20810 |
| | | variable region | | SEQ ID NO: 418 | |
| OC582 | PDGFRβ/VEGF-A | Light chain | Cluster # 997 | US20110177074 | 20811 |
| | | variable region | | SEQ ID NO: 42 | |
| OC583 | PDGFRβ/VEGF-A | Light chain | Cluster # 1458 | US20110177074 | 20812 |
| | | variable region | | SEQ ID NO: 422 | |
| OC584 | PDGFRβ/VEGF-A | Light chain | Cluster # 1476 | US20110177074 | 20813 |
| | | variable region | | SEQ ID NO: 426 | |
| OC585 | PDGFRβ/VEGF-A | Light chain | Cluster # 1479 | US20110177074 | 20814 |
| | | variable region | | SEQ ID NO: 430 | |
| OC586 | PDGFRβ/VEGF-A | Light chain | Cluster # 1035 | US20110177074 | 20815 |
| | | variable region | | SEQ ID NO: 46 | |
| OC587 | PDGFRβ/VEGF-A | Light chain | Cluster # 1228 | US20110177074 | 20816 |
| | | variable region | | SEQ ID NO: 54 | |
| OC588 | PDGFRβ/VEGF-A | Light chain | Cluster # 1230 | US20110177074 | 20817 |
| | | variable region | | SEQ ID NO: 58 | |
| OC589 | PDGFRβ/VEGF-A | Light chain | Cluster # 1231 | US20110177074 | 20818 |
| | | variable region | | SEQ ID NO: 62 | |
| OC590 | PDGFRβ/VEGF-A | Light chain | Cluster # 1236 | US20110177074 | 20819 |
| | | variable region | | SEQ ID NO: 66 | |
| OC591 | PDGFRβ/VEGF-A | Light chain | Cluster # 1238 | US20110177074 | 20820 |
| | | variable region | | SEQ ID NO: 70 | |
| OC592 | PDGFRβ/VEGF-A | Light chain | Cluster # 1244 | US20110177074 | 20821 |
| | | variable region | | SEQ ID NO: 74 | |
| OC593 | PDGFRβ/VEGF-A | Light chain | Cluster # 1245 | US20110177074 | 20822 |
| | | variable region | | SEQ ID NO: 78 | |
| OC594 | PDGFRβ/VEGF-A | Light chain | Cluster # 1299 | US20110177074 | 20823 |
| | | variable region | | SEQ ID NO: 82 | |
| OC595 | PDGFRβ/VEGF-A | Light chain | Cluster # 1312 | US20110177074 | 20824 |
| | | variable region | | SEQ ID NO: 86 | |
| OC596 | PDGFRβ/VEGF-A | Light chain | Cluster # 1314 | US20110177074 | 20825 |
| | | variable region | | SEQ ID NO: 90 | |
| OC597 | PDGFRβ/VEGF-A | Light chain | Cluster # 1317 | US20110177074 | 20826 |
| | | variable region | | SEQ ID NO: 94 | |
| OC598 | PDGFRβ/VEGF-A | Light chain | Cluster # 1322 | US20110177074 | 20827 |
| | | variable region | | SEQ ID NO: 98 | |
| OC599 | PDGFRβ/VEGF-A | Light chain | Cluster # 597 | US20110177074 | 20828 |
| | | variable region | | SEQ ID NO: 6 | |
| OC600 | RGMa | Light chain | AE12-15 | US20140023659 | 20829 |
| | | variable region | | SEQ ID NO: 103 | |
| OC601 | RGMa | Light chain | AE12-20 | US20140023659 | 20830 |
| | | variable region | | SEQ ID NO: 111 | |
| OC602 | RGMa | Light chain | AE12-21 | US20140023659 | 20831 |
| | | variable region | | SEQ ID NO: 119 | |
| OC603 | RGMa | Light chain | AE12-23 | US20140023659 | 20832 |
| | | variable region | | SEQ ID NO: 127 | |
| OC604 | RGMa | Light chain | AE12-2 | US20140023659 | 20833 |
| | | variable region | | SEQ ID NO: 13 | |
| OC605 | RGMa | Light chain | AE12-24 | US20140023659 | 20834 |
| | | variable region | | SEQ ID NO: 135 | |
| OC606 | RGMa | Light chain | AE12-3 | US20140023659 | 20835 |
| | | variable region | | SEQ ID NO: 21 | |
| OC607 | RGMa | Light chain | AE12-4 | US20140023659 | 20836 |
| | | variable region | | SEQ ID NO: 29 | |
| OC608 | RGMa | Light chain | AE12-5 | US20140023659 | 20837 |
| | | variable region | | SEQ ID NO: 37 | |
| OC609 | RGMa | Light chain | AE12-6 | US20140023659 | 20838 |
| | | variable region | | SEQ ID NO: 45 | |
| OC610 | RGMa | Light chain | AE12-1 | US20140023659 | 20839 |
| | | variable region | | SEQ ID NO: 5 | |
| OC611 | RGMa | Light chain | AE12-7 | US20140023659 | 20840 |
| | | variable region | | SEQ ID NO: 53 | |
| OC612 | RGMa | Light chain | AE12-8 | US20140023659 | 20841 |
| | | variable region | | SEQ ID NO: 61 | |
| OC613 | RGMa | Light chain | AE12-13 | US20140023659 | 20842 |
| | | variable region | | SEQ ID NO: 95 | |
| OC614 | S1P4 | Light chain | | WO2015057939 | 20843 |
| | | variable region | | SEQ ID NO: 9 | |
| OC615 | VEGF, C5, | Light chain | NVS73 | US20140186350 | 20844 |
| | Factor P, Factor | variable region | | SEQ ID 120 | |
| | D, EPO, EPOR, | | | | |
| | IL-1β, IL-17A, | | | | |
| | Il-10, TNFα, or | | | | |
| | FGFR2 | | | | |
| OC616 | VEGF, C5, | Light chain | NVS4 | US20140186350 | 20845 |
| | Factor P, Factor | variable region | | SEQ ID 17 | |
| | D, EPO, EPOR, | | | | |
| | IL-1β, IL-17A, | | | | |
| | Il-10, TNFα, or | | | | |
| | FGFR2 | | | | |
| OC617 | VEGF, C5, | Light chain | NVS75 | US20140186350 | 20846 |
| | Factor P, Factor | variable region | | SEQ ID 201 | |
| | D, EPO, EPOR, | | | | |
| | IL-1β, IL-17A, | | | | |
| | Il-10, TNFα, or | | | | |
| | FGFR2 | | | | |
| OC618 | VEGF, C5, | Light chain | NVS70 | US20140186350 | 20847 |
| | Factor P, Factor | variable region | | SEQ ID 49 | |
| | D, EPO, EPOR, | | | | |
| | IL-1β, IL-17A, | | | | |
| | Il-10, TNFα, or | | | | |
| | FGFR2 | | | | |
| OC619 | VEGF, C5, | Light chain | NVS71 | US20140186350 | 20848 |
| | Factor P, Factor | variable region | | SEQ ID 71 | |
| | D, EPO, EPOR, | | | | |
| | IL-1β, IL-17A, | | | | |
| | Il-10, TNFα, or | | | | |
| | FGFR2 | | | | |
| OC620 | VEGF, C5, | Light chain | NVS72 | US20140186350 | 20849 |
| | Factor P, Factor | variable region | | SEQ ID 93 | |
| | D, EPO, EPOR, | | | | |
| | IL-1β, IL-17A, | | | | |
| | Il-10, TNFα, or | | | | |
| | FGFR2 | | | | |
| OC621 | VEGF-A | Light chain | L3 | US20140086829 | 20850 |
| | | variable region | | SEQ ID NO: 8 | |
| OC622 | C5a | Light chain | BNJ364, | US20130224187 | 20851 |
| | | with signal | BNJ367, | SEQ ID 16 | |
| | | peptide | BNJ366, | | |
| | | | BNJ369 | | |
| OC623 | C5a | Light chain | BNJ371, | US20130224187 | 20852 |
| | | with signal | BNJ381 | SEQ ID 35 | |
| | | peptide | | | |
| OC624 | C5a | Light chain | BNJ378, | US20130224187 | 20853 |
| | | with signal | BNJ383 | SEQ ID 39 | |
| | | peptide | | | |
| OC625 | VEGF-A | scFv | L3H6 | US20140086829 | 20854 |
| | | | | SEQ ID NO: 10 | |
| OC626 | VEGF-A | scFv | L3H5 | US20140086829 | 20855 |
| | | | | SEQ ID NO: 12 | |
| OC627 | VEGF-A | scFv | L3H7 | US20140086829 | 20856 |
| | | | | SEQ ID NO: 14 | |
| OC628 | VEGF-A | scFv | Fab L3H6 | US20140086829 | 20857 |
| | | | | SEQ ID 17 | |
| OC629 | VEGF-A | scFv | Fab L3H6 | US20140086829 | 20858 |
| | | | | SEQ ID 18 | |
| OC630 | VEGF-A | scFv | Fab L3H5 | US20140086829 | 20859 |
| | | | | SEQ ID 21 | |
| OC631 | VEGF-A | scFv | Fab L3H5 | US20140086829 | 20860 |
| | | | | SEQ ID 22 | |
| OC632 | VEGF-A | scFv | Fab L3H7 | US20140086829 | 20861 |
| | | | | SEQ ID 25 | |
| OC633 | Annexin IV or a | scoff | B4 | WO2014116880 | 20862 |
| | phospholipid; | | | SEQ ID 17 | |
| | and (b) a | | | | |
| | complement | | | | |
| | inhibitor | | | | |
| OC634 | Annexin IV or a | scFV | B4 | WO2014116880 | 20863 |
| | phospholipid; | | | SEQ ID 18 | |
| | and (b) a | | | | |
| | complement | | | | |
| | inhibitor | | | | |
| OC635 | Annexin IV or a | scFV | C2 | WO2014116880 | 20864 |
| | phospholipid; | | | SEQ ID 37 | |
| | and (b) a | | | | |
| | complement | | | | |
| | inhibitor | | | | |
| OC636 | Annexin IV or a | scFV | C2 | WO2014116880 | 20865 |
| | phospholipid; | | | SEQ ID 38 | |
| | and (b) a | | | | |
| | complement | | | | |
| | inhibitor | | | | |
| OC637 | VEGF, C5, | single chain | NVS78 | US20140186350 | 20866 |
| | Factor P, Factor | | | SEQ ID 146 | |
| | D, EPO, EPOR, | | | | |
| | IL-1β, IL-17A, | | | | |
| | Il-10, TNFα, or | | | | |
| | FGFR2 | | | | |
| OC638 | VEGF, C5, | single chain | NVS78T | US20140186350 | 20867 |
| | Factor P, Factor | | | SEQ ID 147 | |
| | D, EPO, EPOR, | | | | |
| | IL-1β, IL-17A, | | | | |
| | Il-10, TNFα, or | | | | |
| | FGFR2 | | | | |
| OC639 | VEGF, C5, | single chain | NVS90 | US20140186350 | 20868 |
| | Factor P, Factor | | | SEQ ID 148 | |
| | D, EPO, EPOR, | | | | |
| | IL-1β, IL-17A, | | | | |
| | Il-10, TNFα, or | | | | |
| | FGFR2 | | | | |
| OC640 | VEGF, C5, | single chain | NVS90T | US20140186350 | 20869 |
| | Factor P, Factor | | | SEQ ID 149 | |
| | D, EPO, EPOR, | | | | |
| | IL-1β, IL-17A, | | | | |
| | Il-10, TNFα, or | | | | |
| | FGFR2 | | | | |
| OC641 | VEGF, C5, | single chain | NVS79 | US20140186350 | 20870 |
| | Factor P, Factor | | | SEQ ID 150 | |
| | D, EPO, EPOR, | | | | |
| | IL-1β, IL-17A, | | | | |
| | Il-10, TNFα, or | | | | |
| | FGFR2 | | | | |
| OC642 | VEGF, C5, | single chain | NVS79T | US20140186350 | 20871 |
| | Factor P, Factor | | | SEQ ID 151 | |
| | D, EPO, EPOR, | | | | |
| | IL-1β, IL-17A, | | | | |
| | Il-10, TNFα, or | | | | |
| | FGFR2 | | | | |
| OC643 | VEGF, C5, | single chain | NVS91 | US20140186350 | 20872 |
| | Factor P, Factor | | | SEQ ID 152 | |
| | D, EPO, EPOR, | | | | |
| | IL-1β, IL-17A, | | | | |
| | Il-10, TNFα, or | | | | |
| | FGFR2 | | | | |
| OC644 | VEGF, C5, | single chain | NVS91T | US20140186350 | 20873 |
| | Factor P, Factor | | | SEQ ID 153 | |
| | D, EPO, EPOR, | | | | |
| | IL-1β, IL-17A, | | | | |
| | Il-10, TNFα, or | | | | |
| | FGFR2 | | | | |
| OC645 | VEGF, C5, | single chain | NVS80 | US20140186350 | 20874 |
| | Factor P, Factor | | | SEQ ID 154 | |
| | D, EPO, EPOR, | | | | |
| | IL-1β, IL-17A, | | | | |
| | Il-10, TNFα, or | | | | |
| | FGFR2 | | | | |
| OC646 | VEGF, C5, | single chain | NVS80T | US20140186350 | 20875 |
| | Factor P, Factor | | | SEQ ID 156 | |
| | D, EPO, EPOR, | | | | |
| | IL-1β, IL-17A, | | | | |
| | Il-10, TNFα, or | | | | |
| | FGFR2 | | | | |
| OC647 | VEGF, C5, | single chain | NVS83 | US20140186350 | 20876 |
| | Factor P, Factor | | | SEQ ID 165 | |
| | D, EPO, EPOR, | | | | |
| | IL-1β, IL-17A, | | | | |
| | Il-10, TNFα, or | | | | |
| | FGFR2 | | | | |
| OC648 | VEGF, C5, | single chain | NVS83T | US20140186350 | 20877 |
| | Factor P, Factor | | | SEQ ID 166 | |
| | D, EPO, EPOR, | | | | |
| | IL-1β, IL-17A, | | | | |
| | Il-10, TNFα, or | | | | |
| | FGFR2 | | | | |
| OC649 | VEGF, C5, | single chain | NVS84 | US20140186350 | 20878 |
| | Factor P, Factor | | | SEQ ID 167 | |
| | D, EPO, EPOR, | | | | |
| | IL-1β, IL-17A, | | | | |
| | Il-10, TNFα, or | | | | |
| | FGFR2 | | | | |
| OC650 | VEGF, C5, | single chain | NVS84T | US20140186350 | 20879 |
| | Factor P, Factor | | | SEQ ID 168 | |
| | D, EPO, EPOR, | | | | |
| | IL-1β, IL-17A, | | | | |
| | Il-10, TNFα, or | | | | |
| | FGFR2 | | | | |
| OC651 | VEGF, C5, | single chain | NVS85 | US20140186350 | 20880 |
| | Factor P, Factor | | | SEQ ID 169 | |
| | D, EPO, EPOR, | | | | |
| | IL-1β, IL-17A, | | | | |
| | Il-10, TNFα, or | | | | |
| | FGFR2 | | | | |
| OC652 | VEGF, C5, | single chain | NVS85T | US20140186350 | 20881 |
| | Factor P, Factor | | | SEQ ID 170 | |
| | D, EPO, EPOR, | | | | |
| | IL-1β, IL-17A, | | | | |
| | Il-10, TNFα, or | | | | |
| | FGFR2 | | | | |
| OC653 | TGFbeta | single-domain | DOM23h-33 | WO2011012609 | 20882 |
| | | | | SEQ ID 1 | |
| OC654 | TGFbeta | single-domain | DOM23h-439 | WO2011012609 | 20883 |
| | | | | SEQ ID 10 | |
| OC655 | TGFbeta | single-domain | DOM23h-440 | WO2011012609 | 20884 |
| | | | | SEQ ID 11 | |
| OC656 | TGFbeta | single-domain | DOM23h-262- | WO2011012609 | 20885 |
| | | | 6 | SEQ ID 12 | |
| OC657 | TGFbeta | single-domain | DOM23h-262- | WO2011012609 | 20886 |
| | | | 10 | SEQ ID 13 | |
| OC658 | TGFbeta | single-domain | DOM23h-271- | WO2011012609 | 20887 |
| | | | 3 | SEQ ID 14 | |
| OC659 | TGFbeta | single-domain | DOM23h-271- | WO2011012609 | 20888 |
| | | | 7 | SEQ ID 15 | |
| OC660 | TGFbeta | single-domain | DOM23h-271- | WO2011012609 | 20889 |
| | | | 12 | SEQ ID 16 | |
| OC661 | TGFbeta | single-domain | DOM23h-271- | WO2011012609 | 20890 |
| | | | 13 | SEQ ID 17 | |
| OC662 | TGFbeta | single-domain | DOM23h-437- | WO2011012609 | 20891 |
| | | | 4 | SEQ ID 18 | |
| OC663 | TGFbeta | single-domain | DOM23h-437- | WO2011012609 | 20892 |
| | | | 6 | SEQ ID 19 | |
| OC664 | TGFbeta | single-domain | DOM23h-251 | WO2011012609 | 20893 |
| | | | | SEQ ID 2 | |
| OC665 | TGFbeta | single-domain | DOM23h-437- | WO2011012609 | 20894 |
| | | | 8 | SEQ ID 20 | |
| OC666 | TGFbeta | single-domain | DOM23h-437- | WO2011012609 | 20895 |
| | | | 9 | SEQ ID 21 | |
| OC667 | TGFbeta | single-domain | DOM23h-439- | WO2011012609 | 20896 |
| | | | 6 | SEQ ID 22 | |
| OC668 | TGFbeta | single-domain | DOM23h-439- | WO2011012609 | 20897 |
| | | | 8 | SEQ ID 23 | |
| OC669 | TGFbeta | single-domain | DOM23h-262 | WO2011012609 | 20898 |
| | | | | SEQ ID 3 | |
| OC670 | TGFbeta | single-domain | DOM23h-271 | WO2011012609 | 20899 |
| | | | | SEQ ID 4 | |
| OC671 | TGFbeta | single-domain | DOM23h-348 | WO2011012609 | 20900 |
| | | | | SEQ ID 5 | |
| OC672 | TGFbeta | single-domain | DOM23h-435 | WO2011012609 | 20901 |
| | | | | SEQ ID 6 | |
| OC673 | TGFbeta | single-domain | DOM23h-436 | WO2011012609 | 20902 |
| | | | | SEQ ID 7 | |
| OC674 | TGFbeta | single-domain | DOM23h-437 | WO2011012609 | 20903 |
| | | | | SEQ ID 8 | |
| OC675 | TGFbeta | single-domain | DOM23h-438 | WO2011012609 | 20904 |
| | | | | SEQ ID 9 | |
| OC676 | VEGFA | | Brolucizumab, | | 20905 |
| | | | ESBA-1008, | | |
| | | | ESBA1008, | | |

### Systemic Disease Antibodies

In some embodiments, the payload region of the viral particle comprises one or more nucleic acid sequences encoding one or more of the systemic disease payload antibody polypeptides listed in Table 12 (SYS1-SYS73; SEQ ID NO: 20906-20978).

TABLE 12
Systemic Disease Antibodies

| Antibody | | | | Reference | SEQ ID |
|---|---|---|---|---|---|
| No. | Target | Description | Antibody Name | Information | NO |
| SYS1 | integrin αIIbß3, | Chain A, Antibody | Tadocizumab, C4G1, | U.S. Pat. No. 5,777,085 SEQ | 20906 |
| | GPIIb/IIIa | for platelet | YM-337 | ID NO: 23 | |
| | | aggregation | | | |
| SYS2 | integrin αIIbß3, | Chain B, Antibody | Tadocizumab, C4G1, | U.S. Pat. No. 5,777,085 SEQ | 20907 |
| | GPIIb/IIIa | for platelet | YM-337 | ID NO: 12 | |
| | | aggregation | | | |
| SYS3 | | Fab fragment | Tadocizumab | | 20908 |
| SYS4 | | Fab fragment | Tadocizumab | | 20909 |
| SYS5 | | Fusion protein | Sotatercept | | 20910 |
| SYS6 | selectin P | Heavy chain | Inclacumab, LC1004- | | 20911 |
| | | | 002, RO4905417 | | |
| SYS7 | | Heavy chain | Alirocumab | | 20912 |
| SYS8 | | Heavy chain | Abciximab | | 20913 |
| SYS9 | | Heavy chain | Bococizumab | | 20914 |
| SYS10 | | Heavy chain | Evinacumab | | 20915 |
| SYS11 | | Heavy chain | Inclacumab | | 20916 |
| SYS12 | | Heavy chain | Lanadelumab | | 20917 |
| SYS13 | | Heavy chain | Ralpancizumab | | 20918 |
| SYS14 | | Heavy chain | Roledumab | | 20919 |
| SYS15 | CD20 | Heavy Chain, | Idarucizumab | U.S. Pat. No. 8,486,398 SEQ | 20920 |
| | | Antibody for | | ID NO: 35; | |
| | | reversing | | U.S. Pat. No. 8,486,398 SEQ | |
| | | anticoagulation of | | ID NO: 39 | |
| | | dabigatran | | | |
| SYS16 | oxLDL | Heavy chain | Orticumab, BI-204, | | 20921 |
| | | Antibody for acute | MLDL-1278A, R7418, | | |
| | | coronary | RG-7418 | | |
| | | syndrome, | | | |
| | | atherosclerosis | | | |
| SYS17 | | Heavy chain Fab | Idarucizumab | | 20922 |
| | | fragment | | | |
| SYS18 | selectin P | Heavy chain | Inclacumab, LC1004- | U.S. Pat. No. 7,563,441 SEQ | 20923 |
| | | variable region | 002, RO4905417 | ID NO: 4 | |
| SYS19 | oxLDL | Heavy chain | Orticumab, Bi-204, | U.S. Pat. No. 8,318,161 SEQ | 20924 |
| | | variable region, | MLDL-1278A, R7418, | ID NO: 3 | |
| | | Antibody for acute | RG-7418 | | |
| | | coronary | | | |
| | | syndrome, | | | |
| | | atherosclerosis | | | |
| SYS20 | C5 | Heavy Chain | Pexelizumab, 5G1.1-SC | | 20925 |
| | | Variable Region, | | | |
| | | Antibody for | | | |
| | | cardiopulmonary | | | |
| | | bypass, myocardial | | | |
| | | infection, | | | |
| | | h5g1.1VHC + F | | | |
| SYS21 | PCSK9 | Heavy chain | Alirocumab | U.S. Pat. No. 8,062,640 SEQ | 20926 |
| | | variable region, | | ID NO: 90 | |
| | | Antibody for | | | |
| | | cholesterol | | | |
| SYS22 | TNFSF11 | Heavy Chain | Denosumab, Prolia | U.S. Pat. No. 7,364,736; | 20927 |
| | | Variable Region, | | U.S. Pat. No. 8,058,418, | |
| | | Antibody for | | U.S. Pat. No. 8,409,578 | |
| | | osteoporosis | | | |
| SYS23 | TFPI | Heavy chain, | Concizumab, Anti- | U.S. Pat. No. 8,361,469 SEQ | 20928 |
| | | Antibody for | TFPI, NN7415, | ID NO: 24 | |
| | | bleeding, | mab2021 | | |
| SYS24 | PCSK9 | Heavy chain, | Bococizumab | U.S. Pat, No. 8,399,646 SEQ | 20929 |
| | | Antibody for | | ID NO: 15 | |
| | | cardiovascular | | | |
| | | disease | | | |
| SYS25 | PCSK9 | Heavy chain, | Alirocumab | | 20930 |
| | | Antibody for | | | |
| | | cholesterol | | | |
| SYS26 | PCSK9 | Heavy chain, | Ralpancizumab, PF- | | 20931 |
| | | Antibody for | 05335810, RN317 | | |
| | | dyslipidemia, | | | |
| | | Hypercholester- | | | |
| | | olemia | | | |
| SYS27 | F9, F10 | Heavy chain, | Emicizumab, ACE910, | | 20932 |
| | | Antibody for | hBS910 | | |
| | | hematology | | | |
| | | (hemophilia), anti | | | |
| | | 10 | | | |
| SYS28 | F9, F10 | Heavy chain, | Emicizumab, ACE910, | | 20933 |
| | | Antibody for | hBS910 | | |
| | | hematology | | | |
| | | (hemophilia), anti | | | |
| | | F-91 | | | |
| SYS29 | PCSK9 | Heavy chain, | Lodelcizumab, | | 20934 |
| | | Antibody for | LGT209, NVP- | | |
| | | hypercholesterole- | LGT209 | | |
| | | mia | | | |
| SYS30 | PCSK9 | Heavy chain, | Evolocumab | U.S. Pat. No. 8,030,457 | 20935 |
| | | Antibody for | | | |
| | | hyperlipidemia | | | |
| SYS31 | ANGPTL3 | Heavy chain, | Evinacumab, REGN | | 20936 |
| | | Antibody for | 1500 | | |
| | | Hypertriglyceride- | | | |
| | | mia | | | |
| SYS32 | TNFSF11 | Heavy Chain, | Denosumab, Prolia | U.S. Pat. No. 7,364,736; | 20937 |
| | | Antibody for | | U.S. Pat. No. 8,058,418; | |
| | | osteoporosis | | U.S. Pat. No. 8,409,578; | |
| SYS33 | SOST | Heavy chain, | Romosozumab | U.S. Pat. No. 7,592,429 | 20938 |
| | | Antibody for | | U.S. Pat. No. 7,872,106; | |
| | | osteoporosis, | | U.S. Pat. No. 8,003,108; | |
| | | | | U.S. Pat. No. 8,017,120 SEQ | |
| | | | | ID NOs: 147, 145 | |
| SYS34 | SOST | Heavy chain, | Blosozumab | U.S. Pat. No. 7,744,874 SEQ | 20939 |
| | | Antibody for | | ID NO: 3 | |
| | | osteoporosis. | | | |
| SYS35 | TNFSF11 | Heavy chain, | Denosumab, Prolia | U.S. Pat. No. 8,962,807 SEQ | 20940 |
| | | Antibody for | | ID NO: 177; | |
| | | osteoporosis, | | U.S. Pat. No. 7,528,236 SEQ | |
| | | Denosumab | | ID NO: 28 | |
| | | αOPGL-1 | | | |
| SYS36 | RHD | Heavy chain, | Roledumab, LFB-R593, | WO 2010100383 | 20941 |
| | | Antibody for | DMATRIA ™ | | |
| | | prevention of | | | |
| | | fetomaternal | | | |
| | | alloimmunization | | | |
| | | in RhD women | | | |
| SYS37 | CD20 | Heavy Chain, | Idarucizumab | U.S. Pat. No. 8,486,398 SEQ | 20942 |
| | | Antibody for | | ID No: 38; | |
| | | reversing | | U.S. Pat. No. 8,486,398 SEQ | |
| | | anticoagulation of | | ID No: 41; | |
| | | dabigatran | | U.S. Pat. No. 8,486,398 SEQ | |
| | | | | ID No: 36 | |
| SYS38 | | hemophilia | Factor IX-Fc antibody | US20050147618 | 20943 |
| | | | | SEQ ID NO: 23 | |
| SYS39 | | hemophilia | Factor VIII-Fc antibody | WO2011069164 | 20944 |
| | | | | SEQ ID NO: 2 | |
| SYS40 | selectin P | Light chain | Inclacumab, LC1004- | U.S. Pat. No. 8,039,596 SEQ | 20945 |
| | (a5b1) | | 002, RO4905418 | ID NO: 10; | |
| | | | | U.S. Pat. No. 7,432,359 SEQ | |
| | | | | ID NO: 89 | |
| SYS41 | | Light chain | Alirocumab | | 20946 |
| SYS42 | | Light chain | Abciximab | | 20947 |
| SYS43 | | Light chain | Bococizumab | | 20948 |
| SYS44 | | Light chain | Evinacumab | | 20949 |
| SYS45 | | Light chain | Idarucizumab | | 20950 |
| SYS46 | | Light chain | Inclacumab | | 20951 |
| SYS47 | | Light chain | Lanadelumab | | 20952 |
| SYS48 | | Light chain | Ralpancizumab | | 20953 |
| SYS49 | | Light chain | Roledumab | | 20954 |
| SYS50 | ANGPTL3 | Light chain, | Evinacumab, REGN | | 20955 |
| | | Antibody for | 1500 | | |
| | | Hypertriglyceri- | | | |
| | | demia | | | |
| SYS51 | CD41 7E3 | Light chain 1, | Abciximab, c7E3 Fab, | U.S. Pat. No. 5,275,812; | 20956 |
| | | Antibody for | ReoPro | U.S. Pat. No. 5,770,198; | |
| | | preventing blood | | U.S. Pat. No. 5,440,020 | |
| | | clot, ReoPro-Like | | | |
| SYS52 | CD41 7E3 | Light chain 1, | Abciximab, c7E3 Fab, | U.S. Pat. No. 5,275,812; | 20957 |
| | | Antibody for | ReoPro | U.S. Pat. No. 5,770,198; | |
| | | preventing blood | | U.S. Pat. No. 5,440,020 | |
| | | clot, ReoPro-Like | | | |
| SYS53 | selectin P | Light chain | Inclacumab, LC1004- | U.S. Pat. No. 7,563,441 SEQ | 20958 |
| | | variable region | 002, RO4905417 | ID NO: 3 | |
| SYS54 | oxLDL | Light chain | Orticumab, Bi-204, | U.S. Pat. No. 8,318,161 SEQ | 20959 |
| | | variable region, | MLDL-1278A, R7418, | ID NO: 4 | |
| | | Antibody for acute | RG-7418 | | |
| | | coronary | | | |
| | | syndrome, | | | |
| | | atherosclerosis | | | |
| SYS55 | C5 | Light Chain | Pexelizumab, 5G1.1-SC | | 20960 |
| | | Variable Region, | | | |
| | | Antibody for | | | |
| | | cardiopulmonary | | | |
| | | bypass, myocardial | | | |
| | | infection, | | | |
| | | h5g1.1VHC + F | | | |
| SYS56 | PCSK9 | Light chain | Alirocumab | U.S. Pat. No. 8,062,640 SEQ | 20961 |
| | | variable region, | | ID NO: 92 | |
| | | Antibody for | | | |
| | | cholesterol | | | |
| SYS57 | TNFSF11 | Light Chain | Denosumab, Prolia | U.S. Pat. No. 7,364,736; | 20962 |
| | | Variable Region, | | U.S. Pat. No. 8,058,418; | |
| | | Antibody for | | U.S. Pat. No. 8,409,578 | |
| | | osteoporosis | | | |
| SYS58 | oxLDL | Light chain, | Orticumab, BI-204, | | 20963 |
| | | Antibody for acute | MLDL-1278A, R7418, | | |
| | | coronary | RG-7418 | | |
| | | syndrome, | | | |
| | | atherosclerosis | | | |
| SYS59 | PCSK9 | Light chain, | Lodelcizumab, | U.S. Pat. No. 8,710,192 SEQ | 20964 |
| | | Antibody for | LGT209, NVP- | ID NO: 17 | |
| | | blood, | LGT209 | | |
| | | hypercholesterole- | | | |
| | | mia | | | |
| SYS60 | PCSK9 | Light chain, | Bococizumab | U.S. Pat. No. 8,399,646 SEQ | 20965 |
| | | Antibody for | | ID NO: 14 | |
| | | cardiovascular | | | |
| | | disease | | | |
| SYS61 | PCSK9 | Light chain, | Alirocumab | | 20966 |
| | | Antibody for | | | |
| | | cholesterol | | | |
| SYS62 | PCSK9 | Light chain, | Ralpancizumab, PF- | | 20967 |
| | | Antibody for | 05335810, RN317 | | |
| | | dyslipidemia, | | | |
| | | Hypercholesterole- | | | |
| | | mia | | | |
| SYS63 | F9, F10 | Light chain, | Emicizumab, ACE910, | | 20968 |
| | | Antibody for | hBS910 | | |
| | | hematology | | | |
| | | (hemophilia) | | | |
| SYS64 | TFPI | Light chain, | Concizumab, Anti- | U.S. Pat. No. 8,361,469 SEQ | 20969 |
| | | Antibody for | TFPI, NN7415, | ID NO: 21 | |
| | | hemophilia, | mab2021 | | |
| SYS65 | PCSK9 | Light chain, | Evolocumab | U.S. Pat. No. 8,030,457 SEQ | 20970 |
| | | Antibody for | | ID NO: 297 | |
| | | hyperlipidemia | | | |
| SYS66 | TNFSF11 | Light Chain, | Denosumab, Prolia | U.S. Pat. No. 7,364,736; | 20971 |
| | | Antibody for | | U.S. Pat. No. 8,058,418; | |
| | | osteoporosis | | U.S. Pat. No. 8,409,578 | |
| SYS67 | SOST | Light chain, | Romosozumab | U.S. Pat. No. 7,592,429; | 20972 |
| | | Antibody for | | U.S. Pat. No. 7,872,106; | |
| | | osteoporosis, | | U.S. Pat. No. 8,003,108; | |
| | | | | U.S. Pat. No. 8,017,120 SEQ | |
| | | | | ID NOs: 141, 143 | |
| SYS68 | SOST | Light chain, | Blosozumab | U.S. Pat. No. 7,744,874 SEQ | 20973 |
| | | Antibody for | | ID NO: 6 | |
| | | osteoporosis, | | | |
| SYS69 | TNFSF11 | Light chain, | Denosumab, Prolia | U.S. Pat. No. 8,992,925 SEQ | 20974 |
| | | Antibody for | | ID NO: 8; | |
| | | osteoporosis, | | U.S. Pat. No. 7,364,736 SEQ | |
| | | Denosumab | | ID NO: 4 | |
| | | αOPGL-1 | | | |
| SYS70 | RHD | Light chain, | Roledumab, LFB-R593, | WO 2010100383 | 20975 |
| | | Antibody for | DMATRIA ™ | | |
| | | prevention of | | | |
| | | fetomaternal | | | |
| | | alloimmunization | | | |
| | | in RhD women | | | |
| SYS71 | | Peptide | Ecallantide | | 20976 |
| SYS72 | C5 | scFv, Antibody for | Pexelizumab, 5G1.1-SC | | 20977 |
| | | cardiopulmonary | | | |
| | | bypass, myocardial | | | |
| | | infection, h5g1.1 | | | |
| SYS73 | | | Abaloparatide | | 20978 |

### Tau

In some embodiments, the payload region of the viral particle comprises one or more nucleic acid sequences encoding one or more of the payload antibody polypeptides listed in Table 13 (TAU1-TAU1322; SEQ ID NO: 20979-22300).

TABLE 13
Tau Associated Disease Antibodies

| Antibody | | | | | SEQ |
|---|---|---|---|---|---|
| No. | Target | Description | Antibody Name | Reference | ID NO |
| TAU1 | tau | Heavy chain | MC-1 | | 20979 |
| TAU2 | tau | Heavy chain | PHF-1 | | 20980 |
| TAU3 | tau | Heavy chain | IPN002 | | 20981 |
| TAU4 | amyloids | Heavy chain | #118 | WO2010012004 SEQ ID NO: 11 | 20982 |
| TAU5 | amyloids | Heavy chain | #121 | WO2010012004 SEQ ID NO: 13 | 20983 |
| TAU6 | amyloids | Heavy chain | #204 | WO2010012004 SEQ ID NO: 17 | 20984 |
| TAU7 | amyloids | Heavy chain | #205 | WO2010012004 SEQ ID NO: 19 | 20985 |
| TAU8 | NOGO | Heavy chain | H6L13 FL | US20140147435 SEQ ID NO: 27 | 20986 |
| TAU9 | NOGO | Heavy chain | H16L16 FL, | US20140147435 SEQ ID NO: 31 | 20987 |
| | | | H16L18 FL | | |
| TAU10 | NOGO | Heavy chain | H18L16 FL | US20140147435 SEQ ID NO: 33 | 20988 |
| TAU11 | NOGO | Heavy chain | H19L13 FL, | US20140147435 SEQ ID NO: 92 | 20989 |
| | | | H19L16 FL, | | |
| | | | H19L18 FL | | |
| TAU12 | NOGO | Heavy chain | H20L13 FL, | US20140147435 SEQ ID NO: 93 | 20990 |
| | | | H20L16 FL, | | |
| | | | H20L18 FL | | |
| TAU13 | NOGO | Heavy chain | H21L13 FL, | US20140147435 SEQ ID NO: 94 | 20991 |
| | | | H21L16 FL, | | |
| | | | H21L18 FL | | |
| TAU14 | NOGO | Heavy chain | H25L13 FL, | US20140147435 SEQ ID NO: 98 | 20992 |
| | | | H25L16 FL, | | |
| | | | H25L18 FL | | |
| TAU15 | Nogo receptor- | Heavy chain | 5B10 | US20090215691 SEQ ID NO: 16 | 20993 |
| | 1 | | | | |
| TAU16 | Nogo receptor- | Heavy chain | 5B10 | US20090215691 SEQ ID NO: 18 | 20994 |
| | 1 | | | | |
| TAU17 | PrP | Heavy chain | Ab c-120 | WO2014186878 SEQ ID NO: 38 | 20995 |
| TAU18 | PrPC and/or | Heavy chain | | US20150166668 SEQ ID NO: 10 | 20996 |
| | PrPSc | | | | |
| TAU19 | PrPC and/or | Heavy chain | | U.S. Pat. No. 8,852,587 SEQ ID NO: 4 | 20997 |
| | PrPSc | | | | |
| TAU20 | tau | Heavy chain | VH antibody | US20150252102 SEQ ID NO: 93 | 20998 |
| TAU21 | tau | Heavy chain | hAC1-36-3A8 | WO2013151762 SEQ ID NO: 24 | 20999 |
| | | | Ab1 | | |
| TAU22 | tau | Heavy chain | hACI-36-3B8 | WO2013151762 SEQ ID NO: 25 | 21000 |
| | | | Ab1 | | |
| TAU23 | tau | Heavy chain | hACI-36-3A8 | WO2013151762 SEQ ID NO: 26 | 21001 |
| | | | Abl.v2 | | |
| TAU24 | tau | Heavy chain | hACI-36-3A8 | WO2013151762 SEQ ID NO: 27 | 21002 |
| | | | Abl.v3 | | |
| TAU25 | tau | Heavy chain | hAC1-36-3A8 | WO2013151762 SEQ ID NO: 28 | 21003 |
| | | | Ab1.v4 | | |
| TAU26 | tau | Heavy chain | hAC1-36-3B8 | WO2013151762 SEQ ID NO: 29 | 21004 |
| | | | Abl.v2 | | |
| TAU27 | tau | Heavy chain | hACl-36-3B8 | WO2013151762 SEQ ID NO: 30 | 21005 |
| | | | Abl.v3 | | |
| TAU28 | tau | Heavy chain | hACI-36-3B8 | WO2013151762 SEQ ID NO: 31 | 21006 |
| | | | Abl.v4 | | |
| TAU29 | tau | Heavy chain | IPN001 | U.S. Pat. No. 8,980,271 SEQ ID NO: 14 | 21007 |
| TAU30 | tau | Heavy chain | IPN002 | U.S. Pat. No. 8,98,0271 SEQ ID NO: 16 | 21008 |
| TAU31 | tau | Heavy chain | ACI-36-3A8- | US20150175682 SEQ ID NO: 16 | 21009 |
| | | | Ab1 and hACl- | | |
| | | | 36-2B6-Ab1 | | |
| TAU32 | tau | Heavy chain | hACI-36-3A8- | US20150175682 SEQ ID NO: 17 | 21010 |
| | | | Abl and hACl- | | |
| | | | 36-2B6-Ab1 | | |
| TAU33 | tau | Heavy chain | hAC1-36-2B6- | US20150175682 SEQ ID NO: 25 | 21011 |
| | | | Abl (IgG4) | | |
| TAU34 | tau | Heavy chain | hAC1-36-3A8- | US20150175682 SEQ ID NO: 26 | 21012 |
| | | | Abl.v2 (IgG4) | | |
| TAU35 | tau | Heavy chain | hACI-36-3A8- | US20150175682 SEQ ID NO: 27 | 21013 |
| | | | Abl.v3 (IgG1) | | |
| TAU36 | tau | Heavy chain | hACl-36-3A8- | US20150175682 SEQ ID NO: 28 | 21014 |
| | | | Abl.v4 (IgG1 | | |
| | | | N297G) | | |
| TAU37 | tau | Heavy chain | hAC1-36-2B6- | US20150175682 SEQ ID NO: 29 | 21015 |
| | | | Abl.v2 (IgG4) | | |
| TAU38 | tau | Heavy chain | hAC1-36-2B6 | US20150175682 SEQ ID NO: 30 | 21016 |
| | | | Abl.v3 (IgG1) | | |
| TAU39 | tau | Heavy chain | hAC1-36-2B6- | US20150175682 SEQ ID NO: 31 | 21017 |
| | | | Ab1.v4 (IgG1 | | |
| | | | N297G) | | |
| TAU40 | trk-C | Heavy chain | 2250 | U.S. Pat. No. 7,615,383 SEQ ID NO: 42 | 21018 |
| TAU41 | trk-C | Heavy chain | 2253 | U.S. Pat. No. 7,615,383 SEQ ID NO: 43 | 21019 |
| TAU42 | trk-C | Heavy chain | 2256 | U.S. Pat. No. 7,615,383 SEQ ID NO: 44 | 21020 |
| TAU43 | trk-C | Heavy chain | 6.1.2 | U.S. Pat. No. 7,615,383 SEQ ID NO: 45 | 21021 |
| TAU44 | trk-C | Heavy chain | 6.4.1 | U.S. Pat. No. 7,615,383 SEQ ID NO: 46 | 21022 |
| TAU45 | trk-C | Heavy chain | 2345 | U.S. Pat. No. 7,615,383 SEQ ID NO: 47 | 21023 |
| TAU46 | trk-C | Heavy chain | 2349 | U.S. Pat. No. 7,615,383 SEQ ID NO: 48 | 21024 |
| TAU47 | tau | Heavy chain | hAC1-36-3A8- | US20150175682 SEQ ID NO: 14 | 21025 |
| | | constant | Ab1 and hACl- | | |
| | | region | 36-2B6-Abl | | |
| TAU48 | many | Heavy chain | | U.S. Pat. No. 8,053,569 SEQ ID NO: 25 | 21026 |
| | | fusion protein | | | |
| TAU49 | many | Heavy chain | | U.S. Pat. No. 8,053,569 SEQ ID NO: 28 | 21027 |
| | | fusion protein | | | |
| TAU50 | many | Heavy chain | | U.S. Pat. No. 8,053,569 SEQ ID NO: 34 | 21028 |
| | | fusion protein | | | |
| TAU51 | many - growth | Heavy chain | | U.S. Pat. No. 8,053,569 SEQ ID NO: 24 | 21029 |
| | factors (to | fusion protein | | | |
| | increase | | | | |
| | transport | | | | |
| | across BBB) | | | | |
| TAU52 | NOGO | Heavy chain | 2A10 construct | WO2007003421 SEQ ID NO: 79 | 21030 |
| | | humanized | | | |
| | | construct H1 | | | |
| TAU53 | NOGO | Heavy chain | 2A10 construct | WO2007003421 SEQ ID NO: 29 | 21031 |
| | | humanized | | | |
| | | construct H14 | | | |
| TAU54 | NOGO | Heavy chain | 2A10 construct | WO2007003421 SEQ ID NO: 30 | 21032 |
| | | humanized | | | |
| | | construct H15 | | | |
| TAU55 | NOGO | Heavy chain | 2A10 construct | WO2007003421 SEQ ID NO: 31 | 21033 |
| | | humanized | | | |
| | | construct H16 | | | |
| TAU56 | NOGO | Heavy chain | 2A10 construct | WO2007003421 SEQ ID NO: 32 | 21034 |
| | | humanized | | | |
| | | construct H17 | | | |
| TAU57 | NOGO | Heavy chain | 2A10 construct | WO2007003421 SEQ ID NO: 33 | 21035 |
| | | humanized | | | |
| | | construct H18 | | | |
| TAU58 | NOGO | Heavy chain | 2A10 construct | WO2007003421 SEQ ID NO: 92 | 21036 |
| | | humanized | | | |
| | | construct H19 | | | |
| TAU59 | NOGO | Heavy chain | 2A10 construct | WO2007003421 SEQ ID NO: 93 | 21037 |
| | | humanized | | | |
| | | construct H20 | | | |
| TAU60 | NOGO | Heavy chain | 2A10 construct | WO2007003421 SEQ ID NO: 94 | 21038 |
| | | humanized | | | |
| | | construct H21 | | | |
| TAU61 | NOGO | Heavy chain | 2A10 construct | WO2007003421 SEQ ID NO: 95 | 21039 |
| | | humanized | | | |
| | | construct H22 | | | |
| TAU62 | NOGO | Heavy chain | 2A10 construct | WO2007003421 SEQ ID NO: 96 | 21040 |
| | | humanized | | | |
| | | construct H23 | | | |
| TAU63 | NOGO | Heavy chain | 2A10 construct | WO2007003421 SEQ ID NO: 97 | 21041 |
| | | humanized | | | |
| | | construct H24 | | | |
| TAU64 | NOGO | Heavy chain | 2A10 construct | WO2007003421 SEQ ID NO: 98 | 21042 |
| | | humanized | | | |
| | | construct H25 | | | |
| TAU65 | NOGO | Heavy chain | 2A10 construct | WO2007003421 SEQ ID NO: 26 | 21043 |
| | | humanized | | | |
| | | construct H5 | | | |
| TAU66 | NOGO | Heavy chain | 2A10 construct | WO2007003421 SEQ ID NO: 27 | 21044 |
| | | humanized | | | |
| | | construct H6 | | | |
| TAU67 | NOGO | Heavy chain | 2A10 construct | WO2007003421 SEQ ID NO: 28 | 21045 |
| | | humanized | | | |
| | | construct | | | |
| | | H700 | | | |
| TAU68 | RTN4 | Heavy chain | Atinumab | U.S. Pat. No. 8,163,285 SEQ ID NO: 24 | 21046 |
| | (NOGO) | IgG4, | | | |
| | | immunomodu- | | | |
| | | lator | | | |
| TAU69 | tau | Heavy chain | ch4E4 | US20150252102 SEQ ID NO: 20 | 21047 |
| | | mature | | | |
| TAU70 | tau | Heavy chain | ch4E4(N30Q) | US20150252102 SEQ ID NO: 22 | 21048 |
| | | mature | | | |
| TAU71 | NOGO | Heavy chain | 2A10 construct | WO2007003421 SEQ ID NO: 77 | 21049 |
| | | variable | | | |
| | | humanized | | | |
| | | construct H1 | | | |
| TAU72 | NOGO | Heavy chain | 2A10 construct | WO2007003421 SEQ ID NO: 14 | 21050 |
| | | variable | | | |
| | | humanized | | | |
| | | construct H14 | | | |
| TAU73 | NOGO | Heavy chain | 2A10 construct | WO2007003421 SEQ ID NO: 15 | 21051 |
| | | variable | | | |
| | | humanized | | | |
| | | construct H15 | | | |
| TAU74 | NOGO | Heavy chain | 2A10 construct | WO2007003421 SEQ ID NO: 16 | 21052 |
| | | variable | | | |
| | | humanized | | | |
| | | construct H16 | | | |
| TAU75 | NOGO | Heavy chain | 2A10 construct | WO2007003421 SEQ ID NO: 17 | 21053 |
| | | variable | | | |
| | | humanized | | | |
| | | construct H17 | | | |
| TAU76 | NOGO | Heavy chain | 2A10 construct | WO2007003421 SEQ ID NO: 18 | 21054 |
| | | variable | | | |
| | | humanized | | | |
| | | construct H18 | | | |
| TAU77 | NOGO | Heavy chain | 2A10 construct | WO2007003421 SEQ ID NO: 85 | 21055 |
| | | variable | | | |
| | | humanized | | | |
| | | construct H19 | | | |
| TAU78 | NOGO | Heavy chain | 2A10 construct | WO2007003421 SEQ ID NO: 86 | 21056 |
| | | variable | | | |
| | | humanized | | | |
| | | construct H20 | | | |
| TAU79 | NOGO | Heavy chain | 2A10 construct | WO2007003421 SEQ ID NO: 87 | 21057 |
| | | variable | | | |
| | | humanized | | | |
| | | construct H21 | | | |
| TAU80 | NOGO | Heavy chain | 2A10 construct | WO2007003421 SEQ ID NO: 88 | 21058 |
| | | variable | | | |
| | | humanized | | | |
| | | construct H22 | | | |
| TAU81 | NOGO | Heavy chain | 2A10 construct | WO2007003421 SEQ ID NO: 89 | 21059 |
| | | variable | | | |
| | | humanized | | | |
| | | construct H23 | | | |
| TAU82 | NOGO | Heavy chain | 2A10 construct | WO2007003421 SEQ ID NO: 90 | 21060 |
| | | variable | | | |
| | | humanized | | | |
| | | construct H24 | | | |
| TAU83 | NOGO | Heavy chain | 2A10 construct | WO2007003421 SEQ ID NO: 91 | 21061 |
| | | variable | | | |
| | | humanized | | | |
| | | construct H25 | | | |
| TAU84 | NOGO | Heavy chain | 2A10 construct | WO2007003421 SEQ ID NO: 11 | 21062 |
| | | variable | | | |
| | | humanized | | | |
| | | construct H5 | | | |
| TAU85 | NOGO | Heavy chain | 2A10 construct | WO2007003421 SEQ ID NO: 12 | 21063 |
| | | variable | | | |
| | | humanized | | | |
| | | construct H6 | | | |
| TAU86 | NOGO | Heavy chain | 2A10 construct | WO2007003421 SEQ ID NO: 13 | 21064 |
| | | variable | | | |
| | | humanized | | | |
| | | construct | | | |
| | | H700 | | | |
| TAU87 | amyloid | Heavy chain | F11G3 | U.S. Pat. No. 9,125,846 SEQ ID NO: 11 | 21065 |
| | oligomers | variable | | | |
| | | region | | | |
| TAU88 | LPG(lysophos- | Heavy chain | #7 | U.S. Pat. No. 8,591,902 SEQ ID NO: 18 | 21066 |
| | phatidylgluco- | variable | | | |
| | side) | region | | | |
| TAU89 | LPG(lysophos- | Heavy chain | #15 | U.S. Pat. No. 8,591,902 SEQ ID NO: 8 | 21067 |
| | phatidylgluco- | variable | | | |
| | side) | region | | | |
| TAU90 | MAG | Heavy chain | | U.S. Pat. No. 8,071,731 SEQ ID NO: 13 | 21068 |
| | | variable | | | |
| | | region | | | |
| TAU91 | MAG | Heavy chain | | U.S. Pat. No. 8,071,731 SEQ ID NO: 14 | 21069 |
| | | variable | | | |
| | | region | | | |
| TAU92 | MAG | Heavy chain | | U.S. Pat. No. 8,071,731 SEQ ID NO: 15 | 21070 |
| | | variable | | | |
| | | region | | | |
| TAU93 | MAI (myelin | Heavy chain | | WO2013158748 SEQ ID NO: 1 | 21071 |
| | associated | variable | | | |
| | inhibitor) | region | | | |
| TAU94 | MAI (myelin | Heavy chain | | WO2013158748 SEQ ID NO: 17 | 21072 |
| | associated | variable | | | |
| | inhibitor) | region | | | |
| TAU95 | NMDA | Heavy chain | | EP2805972 SEQ ID NO: 43 | 21073 |
| | | variable | | | |
| | | region | | | |
| TAU96 | NOGO | Heavy chain | H5L13, H5L16, | US20140147435 SEQ ID NO: 11 | 21074 |
| | | variable | H5L18, H5L14, | | |
| | | region | H5L15, H5L17 | | |
| | | | H5L6, H5L11 | | |
| TAU97 | NOGO | Heavy chain | H6L13, H6L16, | US20140147435 SEQ ID NO: 12 | 21075 |
| | | variable | H6L18, H6L14, | | |
| | | region | H6L15, H6L17, | | |
| | | | H6L6 | | |
| TAU98 | NOGO | Heavy chain | H700L13, | US20140147435 SEQ ID NO: 13 | 21076 |
| | | variable | H700L16, | | |
| | | region | H700L18, | | |
| | | | H700L14, | | |
| | | | H700L15, | | |
| | | | H700L17, | | |
| | | | H700L6, | | |
| | | | H700L11 | | |
| TAU99 | NOGO | Heavy chain | H14L13, | US20140147435 SEQ ID NO: 14 | 21077 |
| | | variable | H14L16, | | |
| | | region | H14L18, | | |
| | | | H14L14, | | |
| | | | H14L15, | | |
| | | | H14L17, H14L6, | | |
| | | | H14L11 | | |
| TAU100 | NOGO | Heavy chain | H15L13, | US20140147435 SEQ ID NO: 15 | 21078 |
| | | variable | HI5L16, | | |
| | | region | HI5L18, | | |
| | | | HI5L14, | | |
| | | | HI5L15, | | |
| | | | H15L17, H15L6, | | |
| | | | H15L11 | | |
| TAU101 | NOGO | Heavy chain | H16L13, | US20140147435 SEQ ID NO: 16 | 21079 |
| | | variable | H16L16, | | |
| | | region | H16L18, | | |
| | | | H16L14, | | |
| | | | H16L15, | | |
| | | | H16L17, H16L6, | | |
| | | | H16L11 | | |
| TAU102 | NOGO | Heavy chain | H17L13, | US20140147435 SEQ ID NO: 17 | 21080 |
| | | variable | H17L16, | | |
| | | region | H17L18, | | |
| | | | H17L14, | | |
| | | | H17L15, | | |
| | | | H17L17, H17L6, | | |
| | | | H17L11 | | |
| TAU103 | NOGO | Heavy chain | H18L13, | US20140147435 SEQ ID NO: 18 | 21081 |
| | | variable | H18L16, | | |
| | | region | H18L18, | | |
| | | | H18L14, | | |
| | | | H18L15, | | |
| | | | H18L17, H18L6, | | |
| | | | H18L11 | | |
| TAU104 | NOGO | Heavy chain | HIL13, H1L16, | US20140147435 SEQ ID NO: 77 | 21082 |
| | | variable | HIL18, HIL14, | | |
| | | region | HIL15, HIL17, | | |
| | | | H1L6 | | |
| TAU105 | NOGO | Heavy chain | H19L13, | US20140147435 SEQ ID NO: 85 | 21083 |
| | | variable | H19L16, | | |
| | | region | H19L18, | | |
| | | | H19L14, | | |
| | | | H19L15, | | |
| | | | H19L17, H19L6, | | |
| | | | H19L11 | | |
| TAU106 | NOGO | Heavy chain | H20L13, | US20140147435 SEQ ID NO: 86 | 21084 |
| | | variable | H20L16, | | |
| | | region | H20L18, | | |
| | | | H20L14, | | |
| | | | H20L15, | | |
| | | | H20L17, H20L6, | | |
| | | | H20L11 | | |
| TAU107 | NOGO | Heavy chain | H21L13, | US20140147435 SEQ ID NO: 87 | 21085 |
| | | variable | H21L16, | | |
| | | region | H21L18, | | |
| | | | H21L14, | | |
| | | | H21L15, | | |
| | | | H21L17, H21L6, | | |
| | | | H21L11 | | |
| TAU108 | NOGO | Heavy chain | H22L13, | US20140147435 SEQ ID NO: 88 | 21086 |
| | | variable | H22L16, | | |
| | | region | H22L18, | | |
| | | | H22L14, | | |
| | | | H22L15, | | |
| | | | H22L17, H22L6, | | |
| | | | H22L11 | | |
| TAU109 | NOGO | Heavy chain | H23L13, | US20140147435 SEQ ID NO: 89 | 21087 |
| | | variable | H23L16, | | |
| | | region | H23L18, | | |
| | | | H23L14, | | |
| | | | H23L15, | | |
| | | | H23L17, H23L6, | | |
| | | | H23L11 | | |
| TAU110 | NOGO | Heavy chain | H24L13, | US20140147435 SEQ ID NO: 90 | 21088 |
| | | variable | H24L16, | | |
| | | region | H24L18, | | |
| | | | H24L14, | | |
| | | | H24L15, | | |
| | | | H24L17, H24L6, | | |
| | | | H24L11 | | |
| TAU111 | NOGO | Heavy chain | H25L13, | US20140147435 SEQ ID NO: 91 | 21089 |
| | | variable | H25L16, | | |
| | | region | H25L18, | | |
| | | | H25L14, | | |
| | | | H25L15, | | |
| | | | H25L17, H25L6, | | |
| | | | H25L11 | | |
| TAU112 | NOGO | Heavy chain | 2A10 | U.S. Pat. No. 7,988,964 SEQ ID NO: 37 | 21090 |
| | | variable | | | |
| | | region | | | |
| TAU113 | NOGO | Heavy chain | 2C4 | U.S. Pat. No. 7,988,964 SEQ ID NO: 38 | 21091 |
| | | variable | | | |
| | | region | | | |
| TAU114 | NOGO | Heavy chain | 15C3 | U.S. Pat. No. 7,988,964 SEQ ID NO: 39 | 21092 |
| | | variable | | | |
| | | region | | | |
| TAU115 | Nogo-66 | Heavy chain | Antibody clone | US20140065155 SEQ ID NO: 3 | 21093 |
| | | variable | 50 | | |
| | | region | | | |
| TAU116 | Nogo-66 | Heavy chain | Antibody clone | US20140065155 SEQ ID NO: 5 | 21094 |
| | | variable | 51 | | |
| | | region | | | |
| TAU117 | NogoA/NiG | Heavy chain | 6A3-Ig4 | WO2009056509 SEQ ID NO: 24 | 21095 |
| | | variable | | | |
| | | region | | | |
| TAU118 | NogoA/NiG | Heavy chain | 6A3-IgGI | WO2009056509 SEQ ID NO: 4 | 21096 |
| | | variable | | | |
| | | region | | | |
| TAU119 | PrP | Heavy chain | ICSM18VH | US20140294844 SEQ ID NO: 4 | 21097 |
| | | variable | | | |
| | | region | | | |
| TAU120 | PrP | Heavy chain | Ab c-120 | WO2014186878 SEQ ID NO: 40 | 21098 |
| | | variable | | | |
| | | region | | | |
| TAU121 | PrPC and/or | Heavy chain | | US20150166668 SEQ ID NO: 8 | 21099 |
| | PrPSc | variable | | | |
| | | region | | | |
| TAU122 | RGM A | Heavy chain | 5F9.1-GL | US20150183871 SEQ ID NO: 35 | 21100 |
| | | variable | | | |
| | | region | | | |
| TAU123 | RGM A | Heavy chain | 5F9.2-GL | US20150183871 SEQ ID NO: 36 | 21101 |
| | | variable | | | |
| | | region | | | |
| TAU124 | RGM A | Heavy chain | 5F9.3-GL | US20150183871 SEQ ID NO: 37 | 21102 |
| | | variable | | | |
| | | region | | | |
| TAU125 | RGM A | Heavy chain | 5F9.4-GL | US20150183871 SEQ ID NO: 38 | 21103 |
| | | variable | | | |
| | | egion | | | |
| TAU126 | RGM A | Heavy chain | 5F9.5-GL | US20150183871 SEQ ID NO: 39 | 21104 |
| | | variable | | | |
| | | region | | | |
| TAU127 | RGM A | Heavy chain | 5F9.6-GL | US20150183871 SEQ ID NO: 40 | 21105 |
| | | variable | | | |
| | | region | | | |
| TAU128 | RGM A | Heavy chain | 5F9.7-GL | US20150183871 SEQ ID NO: 41 | 21106 |
| | | variable | | | |
| | | region | | | |
| TAU129 | RGM A | Heavy chain | 5F9.8-GL | US20150183871 SEQ ID NO: 42 | 21107 |
| | | variable | | | |
| | | region | | | |
| TAU130 | RGM A | Heavy chain | 5F9.9-GL | US20150183871 SEQ ID NO: 43 | 21108 |
| | | variable | | | |
| | | region | | | |
| TAU131 | RGM A | Heavy chain | h5F9.1, h5F9.1, | US20150183871 SEQ ID NO: 47 | 21109 |
| | | variable | h5F9.1, hF9.1, | | |
| | | region | h5F9.1, hF9.2, | | |
| | | | h5F9.3 | | |
| TAU132 | RGM A | Heavy chain | h5F9.3, h5F9.9, | US20150183871 SEQ ID NO: 53 | 21110 |
| | | variable | h5F9.25 | | |
| | | region | | | |
| TAU133 | RGM A | Heavy chain | h5F9.4, h5F9.10, | US20150183871 SEQ ID NO: 54 | 21111 |
| | | variable | h5F9.26 | | |
| | | region | | | |
| TAU134 | RGMa | Heavy chain | AE12-1 | US20140023659 SEQ ID NO: 1 | 21112 |
| | | variable | | | |
| | | region | | | |
| TAU135 | RGMa | Heavy chain | AE12-20 | US20140023659 SEQ ID NO: 107 | 21113 |
| | | variable | | | |
| | | region | | | |
| TAU136 | RGMa | Heavy chain | AE12-21 | US20140023659 SEQ ID NO: 115 | 21114 |
| | | variable | | | |
| | | region | | | |
| TAU137 | RGMa | Heavy chain | AE12-23 | US20140023659 SEQ ID NO: 123 | 21115 |
| | | variable | | | |
| | | region | | | |
| TAU138 | RGMa | Heavy chain | AE12-24 | US20140023659 SEQ ID NO: 131 | 21116 |
| | | variable | | | |
| | | region | | | |
| TAU139 | RGMa | Heavy chain | AE12-3 | US20140023659 SEQ ID NO: 17 | 21117 |
| | | variable | | | |
| | | region | | | |
| TAU140 | RGMa | Heavy chain | AE12-4 | US20140023659 SEQ ID NO: 25 | 21118 |
| | | variable | | | |
| | | region | | | |
| TAU141 | RGMa | Heavy chain | AE12-5 | US20140023659 SEQ ID NO: 33 | 21119 |
| | | variable | | | |
| | | region | | | |
| TAU142 | RGMa | Heavy chain | AE12-6 | US20140023659 SEQ ID NO: 41 | 21120 |
| | | variable | | | |
| | | region | | | |
| TAU143 | RGMa | Heavy chain | AE12-7 | US20140023659 SEQ ID NO: 49 | 21121 |
| | | variable | | | |
| | | region | | | |
| TAU144 | RGMa | Heavy chain | AE12-8 | US20140023659 SEQ ID NO: 57 | 21122 |
| | | variable | | | |
| | | region | | | |
| TAU145 | RGMa | Heavy chain | AE12-2 | US20140023659 SEQ ID NO: 9 | 21123 |
| | | variable | | | |
| | | region | | | |
| TAU146 | RGMa | Heavy chain | AE12-13 | US20140023659 SEQ ID NO: 91 | 21124 |
| | | variable | | | |
| | | region | | | |
| TAU147 | RGMa | Heavy chain | AE12-15 | US20140023659 SEQ ID NO: 99 | 21125 |
| | | variable | | | |
| | | region | | | |
| TAU148 | tau | Heavy chain | | WO2014100600 SEQ ID NO: 45 | 21126 |
| | | variable | | | |
| | | region | | | |
| TAU149 | tau | Heavy chain | NI-105.24B2 | US20150252102 SEQ ID NO: 13 | 21127 |
| | | variable | | | |
| | | region | | | |
| TAU150 | tau | Heavy chain | NI-105.4A3 | US20150252102 SEQ ID NO: 17 | 21128 |
| | | variable | | | |
| | | region | | | |
| TAU151 | tau | Heavy chain | NI-105.4E4 | US20150252102 SEQ ID NO: 9 | 21129 |
| | | variable | | | |
| | | region | | | |
| TAU152 | tau | Heavy chain | | WO2013041962 SEQ ID NO: 138 | 21130 |
| | | variable | | | |
| | | region | | | |
| TAU153 | tau | Heavy chain | | WO2013041962 SEQ ID NO: 139 | 21131 |
| | | variable | | | |
| | | region | | | |
| TAU154 | tau | Heavy chain | | WO2013041962 SEQ ID NO: 140 | 21132 |
| | | variable | | | |
| | | region | | | |
| TAU155 | tau | Heavy chain | | WO2013041962 SEQ ID NO: 145 | 21133 |
| | | variable | | | |
| | | region | | | |
| TAU156 | tau | Heavy chain | | WO2013041962 SEQ ID NO: 147 | 21134 |
| | | variable | | | |
| | | region | | | |
| TAU157 | tau | Heavy chain | | WO2013041962 SEQ ID NO: 148 | 21135 |
| | | variable | | | |
| | | region | | | |
| TAU158 | tau | Heavy chain | | WO2014100600 SEQ ID NO: 220 | 21136 |
| | | variable | | | |
| | | region | | | |
| TAU159 | tau | Heavy chain | NI-105.17C1 | WO2014100600 SEQ ID NO: 44 | 21137 |
| | | variable | | | |
| | | region | | | |
| TAU160 | tau | Heavy chain | | WO2014100600 SEQ ID NO: 47 | 21138 |
| | | variable | | | |
| | | region | | | |
| TAU161 | tau | Heavy chain | NI-105.6C5 | WO2014100600 SEQ ID NO: 48 | 21139 |
| | | variable | | | |
| | | region | | | |
| TAU162 | tau | Heavy chain | NI-105.29G10 | WO2014100600 SEQ ID NO: 50 | 21140 |
| | | variable | | | |
| | | region | | | |
| TAU163 | tau | Heavy chain | NI-105.6L9 | WO2014100600 SEQ ID NO: 52 | 21141 |
| | | variable | | | |
| | | region | | | |
| TAU164 | tau | Heavy chain | NI-105.40E8 | WO2014100600 SEQ ID NO: 54 | 21142 |
| | | variable | | | |
| | | region | | | |
| TAU165 | tau | Heavy chain | NI-105.48E5 | WO2014100600 SEQ ID NO: 56 | 21143 |
| | | variable | | | |
| | | region | | | |
| TAU166 | tau | Heavy chain | NI-105.6E3 | WO2014100600 SEQ ID NO: 58 | 21144 |
| | | variable | | | |
| | | region | | | |
| TAU167 | tau | Heavy chain | NI-105.22E1 | WO2014100600 SEQ ID NO: 60 | 21145 |
| | | variable | | | |
| | | region | | | |
| TAU168 | tau | Heavy chain | NI-105.26B12 | WO2014100600 SEQ ID NO: 62 | 21146 |
| | | variable | | | |
| | | region | | | |
| TAU169 | tau | Heavy chain | NI-105.12E12 | WO2014100600 SEQ ID NO: 65 | 21147 |
| | | variable | | | |
| | | region | | | |
| TAU170 | tau | Heavy chain | NI-105.60E7 | WO2014100600 SEQ ID NO: 67 | 21148 |
| | | variable | | | |
| | | region | | | |
| TAU171 | tau | Heavy chain | NI-105.14E2 | WO2014100600 SEQ ID NO: 69 | 21149 |
| | | variable | | | |
| | | region | | | |
| TAU172 | tau | Heavy chain | NI-105.39E2 | WO2014100600 SEQ ID NO: 71 | 21150 |
| | | variable | | | |
| | | region | | | |
| TAU173 | tau | Heavy chain | NI-105.19C6 | WO2014100600 SEQ ID NO: 73 | 21151 |
| | | variable | | | |
| | | region | | | |
| TAU174 | tau | Heavy chain | | WO2014100600 SEQ ID NO: 75 | 21152 |
| | | variable | | | |
| | | region | | | |
| TAU175 | tau | Heavy chain | NI-105.9C4 | WO2014100600 SEQ ID NO: 76 | 21153 |
| | | variable | | | |
| | | region | | | |
| TAU176 | tau | Heavy chain | | U.S. Pat. No. 9,304,138 SEQ ID NO: 1 | 21154 |
| | | variable | | | |
| | | region | | | |
| TAU177 | tau | Heavy chain | | U.S. Pat. No. 9,304,138 SEQ ID NO: 2 | 21155 |
| | | variable | | | |
| | | region | | | |
| TAU178 | tau | Heavy chain | | U.S. Pat. No. 9,304,138 SEQ ID NO: 3 | 21156 |
| | | variable | | | |
| | | region | | | |
| TAU179 | tau | Heavy chain | | U.S. Pat. No. 9,304,138 SEQ ID NO: 4 | 21157 |
| | | variable | | | |
| | | region | | | |
| TAU180 | tau | Heavy chain | | U.S. Pat. No. 9,304,138 SEQ ID NO: 5 | 21158 |
| | | variable | | | |
| | | region | | | |
| TAU181 | tau | Heavy chain | | U.S. Pat. No. 9,304,138 SEQ ID NO: 68 | 21159 |
| | | variable | | | |
| | | region | | | |
| TAU182 | tau | Heavy chain | | U.S. Pat. No. 9,304,138 SEQ ID NO: 76 | 21160 |
| | | variable | | | |
| | | region | | | |
| TAU183 | tau | Heavy chain | | U.S. Pat. No. 9,304,138 SEQ ID NO: 88 | 21161 |
| | | variable | | | |
| | | region | | | |
| TAU184 | tau | Heavy chain | | U.S. Pat. No. 9,304,138 SEQ ID NO: 96 | 21162 |
| | | variable | | | |
| | | region | | | |
| TAU185 | tau | Heavy chain | | U.S. Pat. No. 9,304,138 SEQ ID NO: 104 | 21163 |
| | | variable | | | |
| | | region | | | |
| TAU186 | tau | Heavy chain | hAC1-36-3A8- | US20150175682 SEQ ID NO: 7 | 21164 |
| | | variable | Abl and hACl- | | |
| | | region | 36-2B6-Ab1 | | |
| TAU187 | tau | Heavy chain | hACl-36-3A8- | US20150175682 SEQ ID NO: 20 | 21165 |
| | | variable | Ab1.v2. | | |
| | | region | | | |
| TAU188 | tau | Heavy chain | hAC1-36-2B6- | US20150175682 SEQ ID NO: 21 | 21166 |
| | | variable | Ab1.v2 | | |
| | | region | | | |
| TAU189 | tau | Heavy chain | ADx210 | US20140161875 SEQ ID NO: 15 | 21167 |
| | | variable | | | |
| | | region | | | |
| TAU190 | tau | Heavy chain | ADx210 subpart | US20140161875 SEQ ID NO: 17 | 21168 |
| | | variable | | | |
| | | region | | | |
| TAU191 | tau | Heavy chain | ADx215 | US20140161875 SEQ ID NO: 25 | 21169 |
| | | variable | | | |
| | | region | | | |
| TAU192 | tau | Heavy chain | IPN002 variant 1 | U.S. Pat. No. 8,926,974 SEQ ID NO: 36 | 21170 |
| | | variable | | | |
| | | region | | | |
| TAU193 | tau | Heavy chain | IPN002 variant 2 | U.S. Pat. No. 8,926,974 SEQ ID NO: 37 | 21171 |
| | | variable | | | |
| | | region | | | |
| TAU194 | tau | Heavy chain | IPN002 variant 3 | U.S. Pat. No. 8,926,974 SEQ ID NO: 38 | 21172 |
| | | variable | | | |
| | | region | | | |
| TAU195 | tau | Heavy chain | IPN002 variant 4 | U.S. Pat. No. 8,926,974 SEQ ID NO: 39 | 21173 |
| | | variable | | | |
| | | region | | | |
| TAU196 | tau | Heavy chain | PT1 | US20150307600 SEQ ID NO: 35 | 21174 |
| | | variable | | | |
| | | region | | | |
| TAU197 | tau | Heavy chain | PT3 | US20150307600 SEQ ID NO: 37 | 21175 |
| | | variable | | | |
| | | region | | | |
| TAU198 | tau antigen | Heavy chain | ADx202 | WO2015004163 SEQ ID NO: 14 | 21176 |
| | | variable | | | |
| | | region | | | |
| TAU199 | tau pS422 | Heavy chain | antibody | US20110059093 SEQ ID NO: 2 | 21177 |
| | | variable | Mab2.10.3 | | |
| | | region | | | |
| TAU200 | tau pS422 | Heavy chain | Mab 005 | US20110059093 SEQ ID NO: 22 | 21178 |
| | | variable | | | |
| | | region | | | |
| TAU201 | tau pS422 | Heavy chain | Mab 019 | US20110059093 SEQ ID NO: 30 | 21179 |
| | | variable | | | |
| | | region | | | |
| TAU202 | tau pS422 | Heavy chain | Mab 020 | US20110059093 SEQ ID NO: 38 | 21180 |
| | | variable | | | |
| | | region | | | |
| TAU203 | tau pS422 | Heavy chain | Mab 085 | US20110059093 SEQ ID NO: 46 | 21181 |
| | | variable | | | |
| | | region | | | |
| TAU204 | tau pS422 | Heavy chain | Mab 086 | US20110059093 SEQ ID NO: 54 | 21182 |
| | | variable | | | |
| | | region | | | |
| TAU205 | tau pS422 | Heavy chain | Mab 097 | US20110059093 SEQ ID NO: 62 | 21183 |
| | | variable | | | |
| | | region | | | |
| TAU206 | tau | Light chain | MC-1 | | 21184 |
| TAU207 | tau | Light chain | PHF-1 | | 21185 |
| TAU208 | tau | Light chain | IPN002 | | 21186 |
| TAU209 | amyloids | Light chain | #118 | WO2010012004 SEQ ID NO: 12 | 21187 |
| TAU210 | amyloids | Light chain | #121 | WO2010012004 SEQ ID NO: 14 | 21188 |
| TAU211 | amyloids | Light chain | #201 | WO2010012004 SEQ ID NO: 15 | 21189 |
| TAU212 | amyloids | Light chain | #204 | WO2010012004 SEQ ID NO: 16 | 21190 |
| TAU213 | amyloids | Light chain | #205 | WO2010012004 SEQ ID NO: 18 | 21191 |
| TAU214 | NOGO | Light chain | H6L13 FL, | US20140147435 SEQ ID NO: 35 | 21192 |
| | | | H19L13 FL, | | |
| | | | H20L13 FL, | | |
| | | | H21L13 FL, | | |
| | | | H25L13 FL | | |
| TAU215 | NOGO | Light chain | H16L16 FL, | US20140147435 SEQ ID NO: 38 | 21193 |
| | | | H19L16 FL, | | |
| | | | H20L16 FL, | | |
| | | | H21L16 FL, | | |
| | | | H25L16 FL, | | |
| | | | H18L16 FL | | |
| TAU216 | NOGO | Light chain | H16L18 FL, | US20140147435 SEQ ID NO: 40 | 21194 |
| | | | H19L18 FL, | | |
| | | | H20L18 FL, | | |
| | | | H21L18 FL, | | |
| | | | H25L18 FL | | |
| TAU217 | Nogo receptor- | Light chain | 7.00E+11 | US20090215691 SEQ ID NO: 15 | 21195 |
| | 1 | | | | |
| TAU218 | Nogo receptor- | Light chain | 7.00E+11 | US20090215691 SEQ ID NO: 17 | 21196 |
| | 1 | | | | |
| TAU219 | PrP | Light chain | Ab c-120 | WO2014186878 SEQ ID NO: 37 | 21197 |
| TAU220 | PrPC and/or | Light chain | | US20150166668 SEQ ID NO: 9 | 21198 |
| | PrPSc | | | | |
| TAU221 | PrPC and/or | Light chain | | U.S. Pat. No. 8,852,587 SEQ ID NO: 5 | 21199 |
| | PrPSc | | | | |
| TAU222 | tau | Light chain | hAC1-36-3A8 | WO2013151762 SEQ ID NO: 22 | 21200 |
| | | | Abl, hACl-36- | | |
| | | | 3A8 Abl.v2, | | |
| | | | hAC1-36-3A8 | | |
| | | | Abl.v3, hACl- | | |
| | | | 36-3A8 Ab1.v4 | | |
| TAU223 | tau | Light chain | hACl-36-3B8 | WO2013151762 SEQ ID NO: 23 | 21201 |
| | | | Abl, hACl-36- | | |
| | | | 3B8 Abl.v2, | | |
| | | | hAC1-36-3B8 | | |
| | | | Abl.v3, hACl- | | |
| | | | 36-3B8 Abl.v4 | | |
| TAU224 | tau | Light chain | IPN001 | U.S. Pat. No. 8,98,0271 SEQ ID NO: 13 | 21202 |
| TAU225 | tau | Light chain | IPN002 | U.S. Pat. No. 8,980,271 SEQ ID NO: 15 | 21203 |
| TAU226 | tau | Light chain | hAC1-36-3A8- | US20150175682 SEQ ID NO: 18 | 21204 |
| | | | Abl and hACl- | | |
| | | | 36-2B6-Ab | | |
| TAU227 | tau | Light chain | hACl-36-3A8- | US20150175682 SEQ ID NO: 22 | 21205 |
| | | | Ab1 (IgG4), | | |
| | | | hACl-36-3A8- | | |
| | | | Ab1.v2 (IgG4), | | |
| | | | hAC1-36-3A8- | | |
| | | | Ab1.v3 (IgGl), | | |
| | | | and hACl-36- | | |
| | | | 3A8-Abl.v4 | | |
| | | | (IgGl N297G) | | |
| TAU228 | tau | Light chain | hACl-36-2B6- | US20150175682 SEQ ID NO: 23 | 21206 |
| | | | Abl (IgG4), | | |
| | | | hACl-36-2B6- | | |
| | | | Abl.v2 (IgG4), | | |
| | | | hACl-36-2B6- | | |
| | | | Abl.v3 (IgG1), | | |
| | | | and hACl-36- | | |
| | | | 2B6-Ab1.v4 | | |
| | | | (IgG1 N297G) | | |
| TAU229 | tau | Light chain | hAC1-36-3A8- | US20150175682 SEQ ID NO: 24 | 21207 |
| | | | Abl (IgG4) | | |
| TAU230 | trk-C | Light chain | 2250 | U.S. Pat. No. 7,615,383 SEQ ID NO: 49 | 21208 |
| TAU231 | trk-C | Light chain | 2253 | U.S. Pat. No. 7,615,383 SEQ ID NO: 50 | 21209 |
| TAU232 | trk-C | Light chain | 2256 | U.S. Pat. No. 7,615,383 SEQ ID NO: 51 | 21210 |
| TAU233 | trk-C | Light chain | 6.1.2 | U.S. Pat. No. 7,615,383 SEQ ID NO: 52 | 21211 |
| TAU234 | trk-C | Light chain | 6.4.1 | U.S. Pat. No. 7,615,383 SEQ ID NO: 53 | 21212 |
| TAU235 | trk-C | Light chain | 2345 | U.S. Pat. No. 7,615,383 SEQ ID NO: 54 | 21213 |
| TAU236 | trk-C | Light chain | 2349 | U.S. Pat. No. 7,615,383 SEQ ID NO: 55 | 21214 |
| TAU237 | many | Light chain | | U.S. Pat. No. 8,053,569 SEQ ID NO: 31 | 21215 |
| | | fusion protein | | | |
| TAU238 | many | Light chain | | U.S. Pat. No. 8,053,569 SEQ ID NO: 36 | 21216 |
| | | fusion protein | | | |
| TAU239 | NOGO | Light chain | 2A10 construct | WO2007003421 SEQ ID NO: 80 | 21217 |
| | | humanized | | | |
| | | construct L11 | | | |
| TAU240 | NOGO | Light chain | 2A10 construct | WO2007003421 SEQ ID NO: 35 | 21218 |
| | | humanized | | | |
| | | construct L13 | | | |
| TAU241 | NOGO | Light chain | 2A10 construct | WO2007003421 SEQ ID NO: 36 | 21219 |
| | | humanized | | | |
| | | construct L14 | | | |
| TAU242 | NOGO | Light chain | 2A10 construct | WO2007003421 SEQ ID NO: 37 | 21220 |
| | | humanized | | | |
| | | construct L 15 | | | |
| TAU243 | NOGO | Light chain | 2A10 construct | WO2007003421 SEQ ID NO: 38 | 21221 |
| | | humanized | | | |
| | | construct L16 | | | |
| TAU244 | NOGO | Light chain | 2A10 construct | WO2007003421 SEQ ID NO: 39 | 21222 |
| | | humanized | | | |
| | | construct L17 | | | |
| TAU245 | NOGO | Light chain | 2A10 construct | WO2007003421 SEQ ID NO: 40 | 21223 |
| | | humanized | | | |
| | | construct L18 | | | |
| TAU246 | NOGO | Light chain | 2A10 construct | WO2007003421 SEQ ID NO: 34 | 21224 |
| | | humanized | | | |
| | | construct L6 | | | |
| TAU247 | RTN4 | Light chain | Atinumab | U.S. Pat. No. 8,163,285 SEQ ID NO: 25 | 21225 |
| | | IgG4, | | | |
| | | immunomodu- | | | |
| | | lator | | | |
| TAU248 | tau | Light chain | ch4E4 | US20150252102 SEQ ID NO: 21 | 21226 |
| | | mature | | | |
| TAU249 | NOGO | Light chain | 2A10 construct | WO2007003421 SEQ ID NO: 78 | 21227 |
| | | variable | | | |
| | | humanized | | | |
| | | construct L11 | | | |
| TAU250 | NOGO | Light chain | 2A10 construct | WO2007003421 SEQ ID NO: 20 | 21228 |
| | | variable | | | |
| | | humanized | | | |
| | | construct L13 | | | |
| TAU251 | NOGO | Light chain | 2A10 construct | WO2007003421 SEQ ID NO: 21 | 21229 |
| | | variable | | | |
| | | humanized | | | |
| | | construct L 14 | | | |
| TAU252 | NOGO | Light chain | 2A10 construct | WO2007003421 SEQ ID NO: 22 | 21230 |
| | | variable | | | |
| | | humanized | | | |
| | | construct L15 | | | |
| TAU253 | NOGO | Light chain | 2A10 construct | WO2007003421 SEQ ID NO: 23 | 21231 |
| | | variable | | | |
| | | humanized | | | |
| | | construct L16 | | | |
| TAU254 | NOGO | Light chain | 2A10 construct | WO2007003421 SEQ ID NO: 24 | 21232 |
| | | variable | | | |
| | | humanized | | | |
| | | construct L17 | | | |
| TAU255 | NOGO | Light chain | 2A10 construct | WO2007003421 SEQ ID NO: 25 | 21233 |
| | | variable | | | |
| | | humanized | | | |
| | | construct L18 | | | |
| TAU256 | NOGO | Light chain | 2A10 construct | WO2007003421 SEQ ID NO: 19 | 21234 |
| | | variable | | | |
| | | humanized | | | |
| | | construct L6 | | | |
| TAU257 | amyloid | Light chain | F11G3 | U.S. Pat. No. 9,125,846 SEQ ID NO: 12 | 21235 |
| | oligomers | variable | | | |
| | | region | | | |
| TAU258 | LPG(lysophos- | Light chain | #7 | U.S. Pat. No. 8,591,902 SEQ ID NO: 17 | 21236 |
| | phatidylgluco- | variable | | | |
| | side) | region | | | |
| TAU259 | LPG(lysophos- | Light chain | #15 | U.S. Pat. No. 8,591,902 SEQ ID NO: 7 | 21237 |
| | phatidylgluco- | variable | | | |
| | side) | region | | | |
| TAU260 | MAG | Light chain | | U.S. Pat. No. 8,071,731 SEQ ID NO: 16 | 21238 |
| | | variable | | | |
| | | region | | | |
| TAU261 | MAG | Light chain | | U.S. Pat. No. 8,071,731 SEQ ID NO: 17 | 21239 |
| | | variable | | | |
| | | region | | | |
| TAU262 | MAG | Light chain | | U.S. Pat. No. 8,071,731 SEQ ID NO: 18 | 21240 |
| | | variable | | | |
| | | region | | | |
| TAU263 | MAG | Light chain | | U.S. Pat. No. 8,071,731 SEQ ID NO: 19 | 21241 |
| | | variable | | | |
| | | region | | | |
| TAU264 | MAI (myelin | Light chain | | WO2013158748 SEQ ID NO: 11 | 21242 |
| | associated | variable | | | |
| | inhibitor) | region | | | |
| TAU265 | MAI (myelin | Light chain | | WO2013158748 SEQ ID NO: 27 | 21243 |
| | associated | variable | | | |
| | inhibitor) | region | | | |
| TAU266 | NMDA | Light chain | | EP2805972 SEQ ID NO: 44 | 21244 |
| | | variable | | | |
| | | region | | | |
| TAU267 | NOGO | Light chain | HIL6, H5L6, | US20140147435 SEQ ID NO: 19 | 21245 |
| | | variable | H6L6, H14L6, | | |
| | | region | H15L6, H16L6, | | |
| | | | H17L6, H18L6, | | |
| | | | H19L6, H20L6, | | |
| | | | H21L6, H22L6, | | |
| | | | H23L6, H24L6, | | |
| | | | H25L6, H700L6 | | |
| TAU268 | NOGO | Light chain | HIL13, H5L13, | US20140147435 SEQ ID NO: 20 | 21246 |
| | | variable | H6L13, H14L13, | | |
| | | region | H15L13, | | |
| | | | H16L13, | | |
| | | | H17L13, | | |
| | | | H18L13, | | |
| | | | H19L13, | | |
| | | | H20L13, | | |
| | | | H21L13, | | |
| | | | H22L13, | | |
| | | | H23L13, | | |
| | | | H24L13, | | |
| | | | H25L13, | | |
| | | | H700L13 | | |
| TAU269 | NOGO | Light chain | HIL14, H5L14, | US20140147435 SEQ ID NO: 21 | 21247 |
| | | variable | H6L14, H14L14, | | |
| | | region | H15L14, | | |
| | | | H16L14, | | |
| | | | H17L14, | | |
| | | | H18L14, | | |
| | | | H19L14, | | |
| | | | H20L14, | | |
| | | | H21L14, | | |
| | | | H22L14, | | |
| | | | H23L14, | | |
| | | | H24L14, | | |
| | | | H25L14, | | |
| | | | H700L14 | | |
| TAU270 | NOGO | Light chain | HIL15, H5L15, | US20140147435 SEQ ID NO: 22 | 21248 |
| | | variable | H6L15, H14L15, | | |
| | | region | H15L15, | | |
| | | | H16L15, | | |
| | | | H17L15, | | |
| | | | H18L15, | | |
| | | | H19L15, | | |
| | | | H20L15, | | |
| | | | H21L15, | | |
| | | | H22L15, | | |
| | | | H23L15, | | |
| | | | H24L15, | | |
| | | | H25L15, | | |
| | | | H700L15 | | |
| TAU271 | NOGO | Light chain | H1L16, H5L16, | US20140147435 SEQ ID NO: 23 | 21249 |
| | | variable | H6L16, H14L16, | | |
| | | region | H15L16, | | |
| | | | H16L16, | | |
| | | | H17L16, | | |
| | | | H18L16, | | |
| | | | H19L16, | | |
| | | | H20L16, | | |
| | | | H21L16, | | |
| | | | H22L16, | | |
| | | | H23L16, | | |
| | | | H24L16, | | |
| | | | H25L16, | | |
| | | | H700L16 | | |
| TAU272 | NOGO | Light chain | HIL17, H5L17, | US20140147435 SEQ ID NO: 24 | 21250 |
| | | variable | H6L17, H14L17, | | |
| | | region | H15L17, | | |
| | | | H16L17, | | |
| | | | H17L17, | | |
| | | | H18L17, | | |
| | | | H19L17, | | |
| | | | H20L17, | | |
| | | | H21L17, | | |
| | | | H22L17, | | |
| | | | H23L17, | | |
| | | | H24L17, | | |
| | | | H25L17, | | |
| | | | H700L17 | | |
| TAU273 | NOGO | Light chain | H1L18, H5L18, | US20140147435 SEQ ID NO: 25 | 21251 |
| | | variable | H6L18, H14L18, | | |
| | | region | H15L18, | | |
| | | | H16L18, | | |
| | | | H17L18, | | |
| | | | H18L18, | | |
| | | | H19L18, | | |
| | | | H20L18, | | |
| | | | H21L18, | | |
| | | | H22L18, | | |
| | | | H23L18, | | |
| | | | H24L18, | | |
| | | | H25L18, | | |
| | | | H700L18 | | |
| TAU274 | NOGO | Light chain | H5L11, H6L11, | US20140147435 SEQ ID NO: 78 | 21252 |
| | | variable | H14L11, | | |
| | | region | H15L11, | | |
| | | | H16L11, | | |
| | | | H17L11, | | |
| | | | H18L11, | | |
| | | | H19L11, | | |
| | | | H20L11, | | |
| | | | H21L11, | | |
| | | | H22L11, | | |
| | | | H23L11, | | |
| | | | H24L11, | | |
| | | | H25L11, | | |
| | | | H700L11 | | |
| TAU275 | NOGO | Light chain | 2A10 | U.S. Pat. No. 7,988,964 SEQ ID NO: 40 | 21253 |
| | | variable | | | |
| | | region | | | |
| TAU276 | NOGO | Light chain | 2C4 | U.S. Pat. No. 7,988,964 SEQ ID NO: 41 | 21254 |
| | | variable | | | |
| | | region | | | |
| TAU277 | Nogo-66 | Light chain | Antibody clone | U.S. 20140065155 SEQ ID NO: 4 | 21255 |
| | | variable | 50 | | |
| | | region | | | |
| TAU278 | Nogo-66 | Light chain | Antibody clone | US20140065155 SEQ ID NO: 6 | 21256 |
| | | variable | 51 | | |
| | | region | | | |
| TAU279 | NogoA/NiG | Light chain | 6A3-Ig4 | WO2009056509 SEQ ID NO: 25 | 21257 |
| | | variable | | | |
| | | region | | | |
| TAU280 | NogoA/NiG | Light chain | 6A3-IgGI | WO2009056509 SEQ ID NO: 5 | 21258 |
| | | variable | | | |
| | | region | | | |
| TAU281 | PrP | Light chain | Ab c-120 | WO2014186878 SEQ ID NO: 39 | 21259 |
| | | variable | | | |
| | | region | | | |
| TAU282 | PrPC and/or | Light chain | | US20150166668 SEQ ID NO: 7 | 21260 |
| | PrPSc | variable | | | |
| | | region | | | |
| TAU283 | RGM A | Light chain | 5F9.1-GL, | US20150183871 SEQ ID NO: 44 | 21261 |
| | | variable | 5F9.1-GL, | | |
| | | region | 5F9.1-GL, | | |
| | | | 5F9.1-GL, | | |
| | | | 5F9.1-GL, | | |
| | | | 5F9.1-GL, | | |
| | | | 5F9.1-GL, | | |
| | | | 5F9.1-GL, | | |
| | | | 5F9.1-GL, | | |
| | | | 5F9.1-GL, | | |
| | | | h5F9.4, h5F9.11, | | |
| | | | h5F9.12 | | |
| TAU284 | RGM A | Light chain | 5F9.2-GL, | US20150183871 SEQ ID NO: 45 | 21262 |
| | | variable | 5F9.2-GL, | | |
| | | region | 5F9.2-GL, | | |
| | | | 5F9.2-GL, | | |
| | | | 5F9.2-GL, | | |
| | | | 5F9.2-GL, | | |
| | | | 5F9.2-GL, | | |
| | | | 5F9.2-GL, | | |
| | | | 5F9.2-GL, | | |
| | | | 5F9.2-GL, | | |
| | | | h5F9.5, h5F9.19, | | |
| | | | h5F9.20 | | |
| TAU285 | RGM A | Light chain | 5F9.3-GL, | US20150183871 SEQ ID NO: 46 | 21263 |
| | | variable | 5F9.3-GL, | | |
| | | region | 5F9.3-GL, | | |
| | | | 5F9.3-GL, | | |
| | | | 5F9.3-GL, | | |
| | | | 5F9.3-GL, | | |
| | | | 5F9.3-GL, | | |
| | | | 5F9.3-GL, | | |
| | | | 5F9.3-GL, | | |
| | | | 5F9.3-GL, | | |
| | | | hSF9.6. h5F9.21, | | |
| | | | h5F9.22 | | |
| TAU286 | RGM A | Light chain | h5F9.5, h5F9,6, | US20150183871 SEQ ID NO: 48 | 21264 |
| | | variable | h5F9.7, h5F9.8, | | |
| | | region | h5F9.9, h5F9.10 | | |
| TAU287 | RGM A | Light chain | h5F9.11, | US20150183871 SEQ ID NO: 49 | 21265 |
| | | variable | h5F9.19, h5F9.2 | | |
| | | region | 1 | | |
| TAU288 | RGM A | Light chain | h5F9.12, | US20150183871 SEQ ID NO: 50 | 21266 |
| | | variable | h5F9.20, | | |
| | | region | h5F9.22, | | |
| | | | h5F9.23, | | |
| | | | h5F9.25, | | |
| | | | h5F9.25, | | |
| | | | h5F9.26 | | |
| TAU289 | RGM A | Light chain | h5F9.1, h5F9.7, | US20150183871 SEQ ID NO: 51 | 21267 |
| | | variable | h5F9.23 | | |
| | | region | | | |
| TAU290 | RGM A | Light chain | h5F9.2, h5F9.8, | US20150183871 SEQ ID NO: 52 | 21268 |
| | | variable | h5F9.25 | | |
| | | region | | | |
| TAU291 | RGMa | Light chain | AE12-15 | US20140023659 SEQ ID NO: 103 | 21269 |
| | | variable | | | |
| | | region | | | |
| TAU292 | RGMa | Light chain | AE12-20 | US20140023659 SEQ ID NO: 111 | 21270 |
| | | variable | | | |
| | | region | | | |
| TAU293 | RGMa | Light chain | AE12-21 | US20140023659 SEQ ID NO: 119 | 21271 |
| | | variable | | | |
| | | region | | | |
| TAU294 | RGMa | Light chain | AE12-23 | US20140023659 SEQ ID NO: 127 | 21272 |
| | | variable | | | |
| | | region | | | |
| TAU295 | RGMa | Light chain | AE12-2 | US20140023659 SEQ ID NO: 13 | 21273 |
| | | variable | | | |
| | | region | | | |
| TAU296 | RGMa | Light chain | AE12-24 | US20140023659 SEQ ID NO: 135 | 21274 |
| | | variable | | | |
| | | region | | | |
| TAU297 | RGMa | Light chain | AE12-3 | US20140023659 SEQ ID NO: 21 | 21275 |
| | | variable | | | |
| | | region | | | |
| TAU298 | RGMa | Light chain | AE12-4 | US20140023659 SEQ ID NO: 29 | 21276 |
| | | variable | | | |
| | | region | | | |
| TAU299 | RGMa | Light chain | AE12-5 | US20140023659 SEQ ID NO: 37 | 21277 |
| | | variable | | | |
| | | region | | | |
| TAU300 | RGMa | Light chain | AE12-6 | US20140023659 SEQ ID NO: 45 | 21278 |
| | | variable | | | |
| | | region | | | |
| TAU301 | RGMa | Light chain | AE12-1 | US20140023659 SEQ ID NO: 5 | 21279 |
| | | variable | | | |
| | | region | | | |
| TAU302 | RGMa | Light chain | AE12-7 | US20140023659 SEQ ID NO: 53 | 21280 |
| | | variable | | | |
| | | region | | | |
| TAU303 | RGMa | Light chain | AE12-8 | US20140023659 SEQ ID NO: 61 | 21281 |
| | | variable | | | |
| | | region | | | |
| TAU304 | RGMa | Light chain | AE12-13 | US20140023659 SEQ ID NO: 95 | 21282 |
| | | variable | | | |
| | | region | | | |
| TAU305 | tau | Light chain | NI-105.4E4 | US20150252102 SEQ ID NO: 11 | 21283 |
| | | variable | | | |
| | | region | | | |
| TAU306 | tau | Light chain | NI-105.24B2 | US20150252102 SEQ ID NO: 15 | 21284 |
| | | variable | | | |
| | | region | | | |
| TAU307 | tau | Light chain | NI-105.4A3 | US20150252102 SEQ ID NO: 19 | 21285 |
| | | variable | | | |
| | | region | | | |
| TAU308 | tau | Light chain | | WO2013041962 SEQ ID NO: 141 | 21286 |
| | | variable | | | |
| | | region | | | |
| TAU309 | tau | Light chain | | WO2013041962 SEQ ID NO: 142 | 21287 |
| | | variable | | | |
| | | region | | | |
| TAU310 | tau | Light chain | | WO2013041962 SEQ ID NO: 143 | 21288 |
| | | variable | | | |
| | | region | | | |
| TAU311 | tau | Light chain | | WO2013041962 SEQ ID NO: 150 | 21289 |
| | | variable | | | |
| | | region | | | |
| TAU312 | tau | Light chain | | WO2013041962 SEQ ID NO: 152 | 21290 |
| | | variable | | | |
| | | region | | | |
| TAU313 | tau | Light chain | | WO2013041962 SEQ ID NO: 153 | 21291 |
| | | variable | | | |
| | | region | | | |
| TAU314 | tau | Light chain | | WO2014100600 SEQ ID NO: 221 | 21292 |
| | | variable | | | |
| | | region | | | |
| TAU315 | tau | Light chain | | WO2014100600 SEQ ID NO: 222 | 21293 |
| | | variable | | | |
| | | region | | | |
| TAU316 | tau | Light chain | NI-105.17C1 | WO2014100600 SEQ ID NO: 46 | 21294 |
| | | variable | | | |
| | | region | | | |
| TAU317 | tau | Light chain | NI-105.6C5 | WO2014100600 SEQ ID NO: 49 | 21295 |
| | | variable | | | |
| | | region | | | |
| TAU318 | tau | Light chain | NI-105.29G10 | WO2014100600 SEQ ID NO: 51 | 21296 |
| | | variable | | | |
| | | region | | | |
| TAU319 | tau | Light chain | NI-105.6L9 | WO2014100600 SEQ ID NO: 53 | 21297 |
| | | variable | | | |
| | | region | | | |
| TAU320 | tau | Light chain | NI-105.40E8 | WO2014100600 SEQ ID NO: 55 | 21298 |
| | | variable | | | |
| | | region | | | |
| TAU321 | tau | Light chain | NI-105.48E5 | WO2014100600 SEQ ID NO: 57 | 21299 |
| | | variable | | | |
| | | region | | | |
| TAU322 | tau | Light chain | NI-105.6E3 | WO2014100600 SEQ ID NO: 59 | 21300 |
| | | variable | | | |
| | | region | | | |
| TAU323 | tau | Light chain | NI-105.22E1 | WO2014100600 SEQ ID NO: 61 | 21301 |
| | | variable | | | |
| | | region | | | |
| TAU324 | tau | Light chain | | WO2014100600 SEQ ID NO: 63 | 21302 |
| | | variable | | | |
| | | region | | | |
| TAU325 | tau | Light chain | NI-105.26B12 | WO2014100600 SEQ ID NO: 64 | 21303 |
| | | variable | | | |
| | | region | | | |
| TAU326 | tau | Light chain | NI-105.12E12 | WO2014100600 SEQ ID NO: 66 | 21304 |
| | | variable | | | |
| | | region | | | |
| TAU327 | tau | Light chain | NI-105.60E7 | WO2014100600 SEQ ID NO: 68 | 21305 |
| | | variable | | | |
| | | region | | | |
| TAU328 | tau | Light chain | NI-105.14E2 | WO2014100600 SEQ ID NO: 70 | 21306 |
| | | variable | | | |
| | | region | | | |
| TAU329 | tau | Light chain | NI-105.39E2 | WO2014100600 SEQ ID NO: 72 | 21307 |
| | | variable | | | |
| | | region | | | |
| TAU330 | tau | Light chain | NI-105.19C6 | WO2014100600 SEQ ID NO: 74 | 21308 |
| | | variable | | | |
| | | region | | | |
| TAU331 | tau | Light chain | | WO2014100600 SEQ ID NO: 77 | 21309 |
| | | variable | | | |
| | | region | | | |
| TAU332 | tau | Light chain | NI-105.9C4 | WO2014100600 SEQ ID NO: 78 | 21310 |
| | | variable | | | |
| | | region | | | |
| TAU333 | tau | Light chain | IPN002 variant 1 | U.S. Pat. No. 8,926,974 SEQ ID NO: 40 | 21311 |
| | | variable | | | |
| | | region | | | |
| TAU334 | tau | Light chain | IPN002 variant 2 | U.S. Pat. No. 8,926,974 SEQ ID NO: 41 | 21312 |
| | | variable | | | |
| | | region | | | |
| TAU335 | tau | Light chain | IPN002 variant 3 | U.S. Pat. No. 8,926,974 SEQ ID NO: 42 | 21313 |
| | | variable | | | |
| | | region | | | |
| TAU336 | tau | Light chain | IPN002 variant 4 | U.S. Pat. No. 8,926,974 SEQ ID NO: 43 | 21314 |
| | | variable | | | |
| | | region | | | |
| TAU337 | tau | Light chain | PT1 | US20150307600 SEQ ID NO: 36 | 21315 |
| | | variable | | | |
| | | region | | | |
| TAU338 | tau | Light chain | PT3 | US20150307600 SEQ ID NO: 38 | 21316 |
| | | variable | | | |
| | | region | | | |
| TAU339 | tau | Light chain | | U.S. Pat. No. 9,304,138 SEQ ID NO: 6 | 21317 |
| | | variable | | | |
| | | region | | | |
| TAU340 | tau | Light chain | | U.S. Pat. No. 9,304,138 SEQ ID NO: 7 | 21318 |
| | | variable | | | |
| | | region | | | |
| TAU341 | tau | Light chain | | U.S. Pat. No. 9,304,138 SEQ ID NO: 8 | 21319 |
| | | variable | | | |
| | | region | | | |
| TAU342 | tau | Light chain | | U.S. Pat. No. 9,304,138 SEQ ID NO: 9 | 21320 |
| | | variable | | | |
| | | region | | | |
| TAU343 | tau | Light chain | | U.S. Pat. No. 9,304,138 SEQ ID NO: 10 | 21321 |
| | | variable | | | |
| | | region | | | |
| TAU344 | tau | Light chain | | U.S. Pat. No. 9,304,138 SEQ ID NO: 11 | 21322 |
| | | variable | | | |
| | | region | | | |
| TAU345 | tau | Light chain | | U.S. Pat. No. 9,304,138 SEQ ID NO: 69 | 21323 |
| | | variable | | | |
| | | region | | | |
| TAU346 | tau | Light chain | | U.S. Pat. No. 9,304,138 SEQ ID NO: 77 | 21324 |
| | | variable | | | |
| | | region | | | |
| TAU347 | tau | Light chain | | U.S. Pat. No. 9,304,138 SEQ ID NO: 92 | 21325 |
| | | variable | | | |
| | | region | | | |
| TAU348 | tau | Light chain | | U.S. Pat. No. 9,304,138 SEQ ID NO: 97 | 21326 |
| | | variable | | | |
| | | region | | | |
| TAU349 | tau | Light chain | | U.S. Pat. No. 9,304,138 SEQ ID NO: 105 | 21327 |
| | | variable | | | |
| | | region | | | |
| TAU350 | tau | Light chain | | U.S. Pat. No. 9,304,138 SEQ ID NO: 116 | 21328 |
| | | variable | | | |
| | | region | | | |
| TAU351 | tau | Light chain | | U.S. Pat. No. 9,304,138 SEQ ID NO: 118 | 21329 |
| | | variable | | | |
| | | region | | | |
| TAU352 | tau | Light chain | hACl-36-3A8- | US20150175682 SEQ ID NO: 8 | 21330 |
| | | variable | Ab1 | | |
| | | region | | | |
| TAU353 | tau | Light chain | hACl-36-2B6- | US20150175682 SEQ ID NO: 9 | 21331 |
| | | variable | Ab1 | | |
| | | region | | | |
| TAU354 | tau | Light chain | ADx210 | US20140161875 SEQ ID NO: 16 | 21332 |
| | | variable | | | |
| | | region | | | |
| TAU355 | tau | Light chain | ADx210 isoform | US20140161875 SEQ ID NO: 18 | 21333 |
| | | variable | | | |
| | | region | | | |
| TAU356 | tau | Light chain | ADx215 | US20140161875 SEQ ID NO: 26 | 21334 |
| | | variable | | | |
| | | region | | | |
| TAU357 | tau antigen | Light chain | ADx202 | WO2015004163 SEQ ID NO: 9 | 21335 |
| | | variable | | | |
| | | region | | | |
| TAU358 | tau pS422 | Light chain | antibody | US20110059093 SEQ ID NO: 1 | 21336 |
| | | variable | Mab2.10.3 | | |
| | | region | | | |
| TAU359 | tau pS422 | Light chain | Mab 005 | US20110059093 SEQ ID NO: 26 | 21337 |
| | | variable | | | |
| | | region | | | |
| TAU360 | tau pS422 | Light chain | Mab 019 | US20110059093 SEQ ID NO: 34 | 21338 |
| | | variable | | | |
| | | region | | | |
| TAU361 | tau pS422 | Light chain | Mab 020 | US20110059093 SEQ ID NO: 42 | 21339 |
| | | variable | | | |
| | | region | | | |
| TAU362 | tau pS422 | Light chain | Mab 085 | US20110059093 SEQ ID NO: 50 | 21340 |
| | | variable | | | |
| | | region | | | |
| TAU363 | tau pS422 | Light chain | Mab 086 | US20110059093 SEQ ID NO: 58 | 21341 |
| | | variable | | | |
| | | region | | | |
| TAU364 | tau pS422 | Light chain | Mab 097 | US20110059093 SEQ ID NO: 66 | 21342 |
| | | variable | | | |
| | | region | | | |
| TAU365 | PrPC and/or | scFv | | U.S. Pat. No. 8,852,587 SEQ ID NO: 6 | 21343 |
| | PrPSc | | | | |
| TAU366 | amyloid | | M13 g3p | US20150376239 SEQ ID NO: 1 | 21344 |
| | proteins | | | | |
| TAU367 | amyloid | | Construct 5 | US20150376239 SEQ ID NO: 11 | 21345 |
| | proteins | | | | |
| TAU368 | amyloid | | Construct 6 | US20150376239 SEQ ID NO: 13 | 21346 |
| | proteins | | | | |
| TAU369 | amyloid | | fd N2 | US20150376239 SEQ ID NO: 14 | 21347 |
| | proteins | | | | |
| TAU370 | amyloid | | fl N2 | US20150376239 SEQ ID NO: 15 | 21348 |
| | proteins | | | | |
| TAU371 | amyloid | | M13 N2 | US20150376239 SEQ ID NO: 16 | 21349 |
| | proteins | | | | |
| TAU372 | amyloid | | Ike N2 | US20150376239 SEQ ID NO: 17 | 21350 |
| | proteins | | | | |
| TAU373 | amyloid | | 12-2 N2 | US20150376239 SEQ ID NO: 18 | 21351 |
| | proteins | | | | |
| TAU374 | amyloid | | If1 N2 | US20150376239 SEQ ID NO: 19 | 21352 |
| | proteins | | | | |
| TAU375 | amyloid | | fd g3p | US20150376239 SEQ ID NO: 2 | 21353 |
| | proteins | | | | |
| TAU376 | amyloid | | Construct 3 | US20150376239 SEQ ID NO: 20 | 21354 |
| | proteins | | | | |
| TAU377 | amyloid | | Construct 3m | US20150376239 SEQ ID NO: 24 | 21355 |
| | proteins | | g3p portion | | |
| TAU378 | amyloid | | If1 g3p | US20150376239 SEQ ID NO: 29 | 21356 |
| | proteins | | | | |
| TAU379 | amyloid | | fl g3p | US20150376239 SEQ ID NO: 3 | 21357 |
| | proteins | | | | |
| TAU380 | amyloid | | fd g3p | US20150376239 SEQ ID NO: 30 | 21358 |
| | proteins | | | | |
| TAU381 | amyloid | | Construct 8, rs- | US20150376239 SEQ ID NO: 31 | 21359 |
| | proteins | | g3p (If1-NIN2)- | | |
| | | | hlgG1-Fc | | |
| TAU382 | amyloid | | consensus | US20150376239 SEQ ID NO: 4 | 21360 |
| | proteins | | sequence of M13 | | |
| | | | g3p, fd g3p, fl | | |
| | | | g3p | | |
| TAU383 | amyloid | | 12-2 g3p | US20150376239 SEQ ID NO: 5 | 21361 |
| | proteins | | | | |
| TAU384 | amyloid | | Ike g3p | US20150376239 SEQ ID NO: 6 | 21362 |
| | proteins | | | | |
| TAU385 | amyloid | | consensus | US20150376239 SEQ ID NO: 7 | 21363 |
| | proteins | | sequence of 12-2 | | |
| | | | g3p, Ike g3p | | |
| TAU386 | amyloid | | If1 g3p | US20150376239 SEQ ID NO: 8 | 21364 |
| | proteins | | | | |
| TAU387 | amyloid | | Construct 4 | US20150376239 SEQ ID NO: 9 | 21365 |
| | proteins | | | | |
| TAU388 | PrP | | ICSM181c | US20140294844 SEQ ID NO: 6 | 21366 |
| TAU389 | PrPC and/or | | | U.S. Pat. No. 8,852,587 SEQ ID NO: 3 | 21367 |
| | PrPSc | | | | |
| TAU390 | tau | | | US20140302046 SEQ ID NO: 103 | 21368 |
| TAU391 | B-amyloid | Heavy chain | 1B 1-40 | US20100323905 SEQ ID NO: 92 | 21369 |
| | | variable | | | |
| | | region | | | |
| | | antibody | | | |
| TAU392 | B-amyloid | Heavy chain | 3A 1-42 | US20100323905 SEQ ID NO: 94 | 21370 |
| | | variable | | | |
| | | region | | | |
| | | antibody | | | |
| TAU393 | B-amyloid | Heavy chain | FC5 | US20100323905 SEQ ID NO: 96 | 21371 |
| | | variable | | | |
| | | region | | | |
| | | antibody | | | |
| TAU394 | Tau | Chain A, | | Cehlar, O. et al., ″Structure Of Tau | 21372 |
| | | Structure Of | | Peptide In Complex With Tau5 | |
| | | Tau Peptide | | Antib Fragment″, unpublished, | |
| | | In Complex | | 4TQE_A | |
| | | With Tau5 | | | |
| | | Antibody Fab | | | |
| | | Fragment | | | |
| TAU395 | Tau | Chain A and | | Shih, H. H., et al., An ultra-specific | 21373 |
| | | B, Structure | | avian antibody to phosphorylated | |
| | | Of The Anti- | | tau protein reveals a unique | |
| | | ptau Fab | | mechanism for phosphoepitope | |
| | | (pt231/ | | recognition″, J. Biol. Chem. 287 | |
| | | ps235_1) In | | (53), 44425-44434 (2012), | |
| | | Complex | | Accession number 4GLR_A and | |
| | | With | | 4GLR_B | |
| | | Phosphoepi- | | | |
| | | tope | | | |
| | | Pt231/ps235 | | | |
| TAU396 | Tau | Chain P | At8 Fab | Malia, T. J. et al, ″Epitope mapping | 21374 |
| | | Anti-tau At8 | | and structural basis for the | |
| | | Fab With | | recognition of phosphorylated tau | |
| | | Doubly | | by the anti-tau antibody AT8″, | |
| | | Phosphorylat- | | Proteins 84 (4), 427-434 (2016), | |
| | | ed Tau | | Accession number 5E2V_P | |
| | | Peptide | | | |
| TAU397 | Tau | Chain P, | At8 Fab | Malia, T. J. et al, ″Epitope mapping | 21375 |
| | | Anti-tau At8 | | and structural basis for the | |
| | | Fab With | | recognition of phosphorylated tau | |
| | | Triply | | by the anti-tau antibody AT8″, | |
| | | Phosphorylat- | | Proteins 84 (4), 427-434 (2016), | |
| | | ed Tau | | Accession number 5E2W_P | |
| | | Peptide | | | |
| TAU398 | Tau | Chain P, X- | Rb86 | Bujotzek, A. et al, ″VH-VL | 21376 |
| | | ray Structure | | orientation prediction for antibody | |
| | | Of The Fab | | humanization candidate selection: | |
| | | Fragment Of | | A case study″, MAbs 8 (2), 288- | |
| | | The Anti Tau | | 305 (2016), Accession number | |
| | | Antibody | | 5DMG_P, 5DMG_X, 5DMG_Z | |
| | | Rb86 In | | | |
| | | Complex | | | |
| | | With The | | | |
| | | Phosphorylat- | | | |
| | | ed Tau | | | |
| | | Peptide (416- | | | |
| | | 430) | | | |
| TAU399 | Tau | Heavy chain | CDC8E8 VH | WO2016079597 SEQ ID NO: 9; | 21377 |
| | | | | US20150050215 SEQ ID NO: 138 | |
| TAU400 | Tau | Heavy chain | RHA - IgG1 | WO2016079597 SEQ ID NO: 28 | 21378 |
| TAU401 | Tau | Heavy chain | RHB - IgG1 | WO2016079597 SEQ ID NO: 29 | 21379 |
| TAU402 | Tau | Heavy chain | RHC - IgGI | WO2016079597 SEQ ID NO: 30 | 21380 |
| TAU403 | Tau | Heavy chain | RHD - IgG1 | WO2016079597 SEQ ID NO: 31 | 21381 |
| TAU404 | Tau | Heavy chain | RHE - IgG1 | WO2016079597 SEQ ID NO: 32 | 21382 |
| TAU405 | Tau | Heavy chain | RHF - IgG1 | WO2016079597 SEQ ID NO: 33 | 21383 |
| TAU406 | Tau | Heavy chain | RHG - IgG1 | WO2016079597 SEQ ID NO: 34 | 21384 |
| TAU407 | Tau | Heavy chain | RHH - IgG1 | WO2016079597 SEQ ID NO: 35 | 21385 |
| TAU408 | Tau | Heavy chain | RHI ~ IgG1 | WO2016079597 SEQ ID NO: 36 | 21386 |
| TAU409 | Tau | Heavy chain | RHJ - IgG1 | WO2016079597 SEQ ID NO: 37 | 21387 |
| TAU410 | Tau | Heavy chain | RHK - IgG1 | WO2016079597 SEQ ID NO: 38 | 21388 |
| TAU411 | Tau | Heavy chain | RHL - IgG1 | WO2016079597 SEQ ID NO: 39 | 21389 |
| TAU412 | Tau | Heavy chain | RHM - IgG1 | WO2016079597 SEQ ID NO: 40 | 21390 |
| TAU413 | Tau | Heavy chain | CDC8E8 - IgG1 | WO2016079597 SEQ ID NO: 41 | 21391 |
| TAU414 | Tau | Heavy chain | mouse DC8E8 - | WO2016079597 SEQ ID NO: 42 | 21392 |
| | | | IgG1 | | |
| TAU415 | Tau | Heavy chain | RHA - IgG4 | WO2016079597 SEQ ID NO: 43 | 21393 |
| TAU416 | Tau | Heavy chain | RHB - IgG4 | WO2016079597 SEQ ID NO: 44 | 21394 |
| TAU417 | Tau | Heavy chain | RHC - IgG4 | WO2016079597 SEQ ID NO: 45 | 21395 |
| TAU418 | Tau | Heavy chain | RHD - IgG4 | WO2016079597 SEQ ID NO: 46 | 21396 |
| TAU419 | Tau | Heavy chain | RHE - IgG4 | WO2016079597 SEQ ID NO: 47 | 21397 |
| TAU420 | Tau | Heavy chain | RHF - 1gG4 | WO2016079597 SEQ ID NO: 48 | 21398 |
| TAU421 | Tau | Heavy chain | RHG - IgG4 | WO2016079597 SEQ ID NO: 49 | 21399 |
| TAU422 | Tau | Heavy chain | RHH - IgG4 | WO2016079597 SEQ ID NO: 50 | 21400 |
| TAU423 | Tau | Heavy chain | RHI - IgG4 | WO2016079597 SEQ ID NO: 51 | 21401 |
| TAU424 | Tau | Heavy chain | RHJ - IgG4 | WO2016079597 SEQ ID NO: 52 | 21402 |
| TAU425 | Tau | Heavy chain | RHK ~ IgG4 | WO2016079597 SEQ ID NO: 53 | 21403 |
| TAU426 | Tau | Heavy chain | RHL - IgG4 | WO2016079597 SEQ ID NO: 54 | 21404 |
| TAU427 | Tau | Heavy chain | RHM - IgG4 | WO2016079597 SEQ ID NO: 55 | 21405 |
| TAU428 | Tau | Heavy chain | cDC8E8 - IgG4 | WO2016079597 SEQ ID NO: 56 | 21406 |
| TAU429 | Tau | Heavy chain | DC8E8 | WO2016079597 SEQ ID NO: 90 | 21407 |
| TAU430 | Tau | Heavy chain | cDC8E8 | WO2016079597 SEQ ID NO: 92 | 21408 |
| TAU431 | Tau | Heavy chain | OptiDC8E8 | WO2016079597 SEQ ID NO: 94 | 21409 |
| TAU432 | Tau | Heavy chain | RHA | WO2016079597 SEQ ID NO: 96 | 21410 |
| TAU433 | Tau | Heavy chain | RHB | WO2016079597 SEQ ID NO: 97 | 21411 |
| TAU434 | Tau | Heavy chain | RHC | WO2016079597 SEQ ID NO: 98 | 21412 |
| TAU435 | Tau | Heavy chain | RHD | WO2016079597 SEQ ID NO: 99 | 21413 |
| TAU436 | Tau | Heavy chain | RHE | WO2016079597 SEQ ID NO: 100 | 21414 |
| TAU437 | Tau | Heavy chain | RHF | WO2016079597 SEQ ID NO: 101 | 21415 |
| TAU438 | Tau | Heavy chain | RHG | WO2016079597 SEQ ID NO: 102 | 21416 |
| TAU439 | Tau | Heavy chain | RHH | WO2016079597 SEQ ID NO: 103 | 21417 |
| TAU440 | Tau | Heavy chain | RHI | WO2016079597 SEQ ID NO: 104 | 21418 |
| TAU441 | Tau | Heavy chain | RHJ | WO2016079597 SEQ ID NO: 105 | 21419 |
| TAU442 | Tau | Heavy chain | RHK | WO2016079597 SEQ ID NO: 106 | 21420 |
| TAU443 | Tau | Heavy chain | RHL | WO2016079597 SEQ ID NO: 107 | 21421 |
| TAU444 | Tau | Heavy chain | RHM | WO2016079597 SEQ ID NO: 108 | 21422 |
| TAU445 | Tau | Heavy chain | RHA - IgG1 | WO2016079597 SEQ ID NO: 111 | 21423 |
| TAU446 | Tau | Heavy chain | RHB - IgG1 | WO2016079597 SEQ ID NO: 112 | 21424 |
| TAU447 | Tau | Heavy chain | RHC - IgG1 | WO2016079597 SEQ ID NO: 113 | 21425 |
| TAU448 | Tau | Heavy chain | RHD - IgG1 | WO2016079597 SEQ ID NO: 114 | 21426 |
| TAU449 | Tau | Heavy chain | RHE - IgG1 | WO2016079597 SEQ ID NO: 115 | 21427 |
| TAU450 | Tau | Heavy chain | RHF - IgG1 | WO2016079597 SEQ ID NO: 116 | 21428 |
| TAU451 | Tau | Heavy chain | RHG - IgG1 | WO2016079597 SEQ ID NO: 117 | 21429 |
| TAU452 | Tau | Heavy chain | RHH - IgG1 | WO2016079597 SEQ ID NO: 118 | 21430 |
| TAU453 | Tau | Heavy chain | RHI - IgGI | WO2016079597 SEQ ID NO: 119 | 21431 |
| TAU454 | Tau | Heavy chain | RHJ - IgGI | WO2016079597 SEQ ID NO: 120 | 21432 |
| TAU455 | Tau | Heavy chain | RHK - IgG1 | WO2016079597 SEQ ID NO: 121 | 21433 |
| TAU456 | Tau | Heavy chain | RHL - IgG1 | WO2016079597 SEQ ID NO: 122 | 21434 |
| TAU457 | Tau | Heavy chain | RHM - IgG1 | WO2016079597 SEQ ID NO: 123 | 21435 |
| TAU458 | Tau | Heavy chain | cDC8E8 - IgG1 | WO2016079597 SEQ ID NO: 124 | 21436 |
| TAU459 | Tau | Heavy chain | mouse DC8E8 - | WO2016079597 SEQ ID NO: 125 | 21437 |
| | | | IgG1 | | |
| TAU460 | Tau | Heavy chain | codon opt mouse | WO2016079597 SEQ ID NO: 126 | 21438 |
| | | | DC8E8 | | |
| TAU461 | Tau | Heavy chain | RHA - IgG4 | WO2016079597 SEQ ID NO: 127 | 21439 |
| TAU462 | Tau | Heavy chain | RHB - IgG4 | WO2016079597 SEQ ID NO: 128 | 21440 |
| TAU463 | Tau | Heavy chain | RHC - IgG4 | WO2016079597 SEQ ID NO: 129 | 21441 |
| TAU464 | Tau | Heavy chain | RHD - IgG4 | WO2016079597 SEQ ID NO: 130 | 21442 |
| TAU465 | Tau | Heavy chain | RHE - IgG4 | WO2016079597 SEQ ID NO: 131 | 21443 |
| TAU466 | Tau | Heavy chain | RHF - IgG4 | WO2016079597 SEQ ID NO: 132 | 21444 |
| TAU467 | Tau | Heavy chain | RHG - IgG4 | WO2016079597 SEQ ID NO: 133 | 21445 |
| TAU468 | Tau | Heavy chain | RHH - IgG4 | WO2016079597 SEQ ID NO: 134 | 21446 |
| TAU469 | Tau | Heavy chain | RHI - IgG4 | WO2016079597 SEQ ID NO: 135 | 21447 |
| TAU470 | Tau | Heavy chain | RHJ - 1gG4 | WO2016079597 SEQ ID NO: 136 | 21448 |
| TAU471 | Tau | Heavy chain | RHK - IgG4 | WO2016079597 SEQ ID NO: 137 | 21449 |
| TAU472 | Tau | Heavy chain | RHL - IgG4 | WO2016079597 SEQ ID NO: 138 | 21450 |
| TAU473 | Tau | Heavy chain | RHM - IgG4 | WO2016079597 SEQ ID NO: 139 | 21451 |
| TAU474 | Tau | Heavy chain | cDC8E8 - IgG4 | WO2016079597 SEQ ID NO: 140 | 21452 |
| TAU475 | Tau | Heavy chain | | U.S. Pat. No. 8,697,076 SEQ ID NO: 12 | 21453 |
| TAU476 | Tau | Heavy chain | 5202.4 | US20160024193 SEQ ID NO: 63 | 21454 |
| TAU477 | Tau | Heavy chain | | US20160031977 SEQ ID NO: 22 | 21455 |
| TAU478 | Tau | Heavy chain | | US20160031977 SEQ ID NO: 24 | 21456 |
| TAU479 | Tau | Heavy chain | | US20160031977 SEQ ID NO: 26 | 21457 |
| TAU480 | Tau | heavy chain | ch4A3-mlgGl- | US20150344553 SEQ ID NO: 213 | 21458 |
| | | | Agly | | |
| TAU481 | Tau | heavy chain | ch4E4(N30Q)- | US20150344553 SEQ ID NO: 214 | 21459 |
| | | | mIgG1-Agly | | |
| TAU482 | Tau | heavy chain | ch6C5-mIgG1- | US20150344553 SEQ ID NO: 215 | 21460 |
| | | | Agly | | |
| TAU483 | Tau | heavy chain | ch17C1-mlgG1- | US20150344553 SEQ ID NO: 216 | 21461 |
| | | | Agly | | |
| TAU484 | Tau | Heavy chain | human NI- | US20150344553 SEQ ID NO: 218 | 21462 |
| | | | 105.40E8 | | |
| | | | (R104W)-hIgG1 | | |
| TAU485 | Tau | Heavy chain | NI- | US20150344553 SEQ ID NO: 43; | 21463 |
| | | | 105.4E4(N30Q) | U.S. Pat. No. 8,940,272 SEQ ID NO: 93 | |
| TAU486 | Tau | Heavy chain | | US20150050215 SEQ ID NO: 140 | 21464 |
| TAU487 | Tau | Heavy chain | | US20150050215 SEQ ID NO: 142 | 21465 |
| TAU488 | Tau | Heavy chain | pT231/pS235 | WO2014016737 SEQ ID NO: 70 | 21466 |
| TAU489 | Tau | heavy chain | ch40E8(R104W) | US20150344553 SEQ ID NO: 208 | 21467 |
| | | (mouse | | | |
| | | IgG2a) | | | |
| TAU490 | Tau | heavy chain | ch17C1 | US20150344553 SEQ ID NO: 203 | 21468 |
| | | (mouse | | | |
| | | IgG2a) | | | |
| TAU491 | Tau | heavy chain | ch6C5 | US20150344553 SEQ ID NO: 205 | 21469 |
| | | (mouse | | | |
| | | IgG2a) | | | |
| TAU492 | Tau | heavy chain | ch40E8 | US20150344553 SEQ ID NO: 207 | 21470 |
| | | (mouse | | | |
| | | IgG2a) | | | |
| TAU493 | Tau | heavy chain | ch6E3 | US20150344553 SEQ ID NO: 210 | 21471 |
| | | (mouse | | | |
| | | IgG2a) | | | |
| TAU494 | Tau | heavy chain | | WO2016079597 SEQ ID NO: 172 | 21472 |
| | | constant | | | |
| | | region | | | |
| TAU495 | Tau | heavy chain | | WO2016079597 SEQ ID NO: 173 | 21473 |
| | | constant | | | |
| | | region | | | |
| TAU496 | Tau | Heavy chain | | WO2015197823 SEQ ID NO: 83 | 21474 |
| | | constant | | | |
| | | region, IgGl | | | |
| TAU497 | Tau | Heavy chain | ch4E4(N30Q) | U.S. Pat. No. 8,940,272 SEQ ID NO: 22 | 21475 |
| | | mature | | | |
| | | (mouse | | | |
| | | IgG2a) | | | |
| TAU498 | Tau | Heavy chain | ch4E4 | U.S. Pat. No. 8,940,272 SEQ ID NO: 20 | 21476 |
| | | mature | | | |
| | | (mouse | | | |
| | | IgG2a) | | | |
| TAU499 | Tau | Heavy chain | ch4E4 | US20150344553 SEQ ID NO: 20 | 21477 |
| | | mature | | | |
| | | (mouse | | | |
| | | IgG2a) | | | |
| TAU500 | Tau | Heavy chain | ch4E4(N30Q) | US20150344553 SEQ ID NO: 22 | 21478 |
| | | mature | | | |
| | | (mouse | | | |
| | | IgG2a) | | | |
| TAU501 | Tau | Heavy chain | NI-105.4A3-VH | US20150344553 SEQ ID NO: 17; | 21479 |
| | | variable | | U.S. Pat. No. 8,940,272 SEQ ID NO: 17 | |
| TAU502 | Tau | Heavy chain | NI-105.24B2- | US20150344553 SEQ ID NO: 13; | 21480 |
| | | variable | VH | U.S. Pat. No. 8,940,272 SEQ ID NO: 13 | |
| TAU503 | Tau | Heavy chain | NI-105.4E4-VH | US20150344553 SEQ ID NO: 9; | 21481 |
| | | variable | | U.S. Pat. No. 8,940,272 SEQ ID NO: 9 | |
| TAU504 | Tau | Heavy chain | | US20150307600 SEQ ID NO: 35 | 21482 |
| | | variable | | | |
| TAU505 | Tau | Heavy chain | | US20150307600 SEQ ID NO: 37 | 21483 |
| | | variable | | | |
| TAU506 | Tau | Heavy chain | RHA | WO2016079597 SEQ ID NO: 13 | 21484 |
| | | variable | | | |
| | | region | | | |
| TAU507 | Tau | Heavy chain | RHB | WO2016079597 SEQ ID NO: 14 | 21485 |
| | | variable | | | |
| | | region | | | |
| TAU508 | Tau | Heavy chain | RHC | WO2016079597 SEQ ID NO: 15 | 21486 |
| | | variable | | | |
| | | region | | | |
| TAU509 | Tau | Heavy chain | RHD | WO2016079597 SEQ ID NO: 16 | 21487 |
| | | variable | | | |
| | | region | | | |
| TAU510 | Tau | Heavy chain | RHE | WO2016079597 SEQ ID NO: 17 | 21488 |
| | | variable | | | |
| | | region | | | |
| TAU511 | Tau | Heavy chain | RHF | WO2016079597 SEQ ID NO: 18 | 21489 |
| | | variable | | | |
| | | region | | | |
| TAU512 | Tau | Heavy chain | RHG | WO2016079597 SEQ ID NO: 19 | 21490 |
| | | variable | | | |
| | | region | | | |
| TAU513 | Tau | Heavy chain | RHH | WO2016079597 SEQ ID NO: 20 | 21491 |
| | | variable | | | |
| | | region | | | |
| TAU514 | Tau | Heavy chain | RHI | WO2016079597 SEQ ID NO: 21 | 21492 |
| | | variable | | | |
| | | region | | | |
| TAU515 | Tau | Heavy chain | RHJ | WO2016079597 SEQ ID NO: 22 | 21493 |
| | | variable | | | |
| | | region | | | |
| TAU516 | Tau | Heavy chain | RHK | WO2016079597 SEQ ID NO: 23 | 21494 |
| | | variable | | | |
| | | region | | | |
| TAU517 | Tau | Heavy chain | RHL | WO2016079597 SEQ ID NO: 24 | 21495 |
| | | variable | | | |
| | | region | | | |
| TAU518 | Tau | Heavy chain | RHM | WO2016079597 SEQ ID NO: 25 | 21496 |
| | | variable | | | |
| | | region | | | |
| TAU519 | Tau | Heavy chain | | U.S. Pat. No. 8,697,076 SEQ ID NO: 7 | 21497 |
| | | variable | | | |
| | | region | | | |
| TAU520 | Tau | Heavy chain | | US20160024193 SEQ ID NO: 58 | 21498 |
| | | variable | | and 62 | |
| | | region | | | |
| TAUS21 | Tau | Heavy chain | 16B5 | US20160031976 SEQ ID NO: 10 | 21499 |
| | | variable | | | |
| | | region | | | |
| TAU522 | Tau | Heavy chain | NI-105.17C1 | US20150344553 SEQ ID NO: 45 | 21500 |
| | | variable | | | |
| | | region | | | |
| TAU523 | Tau | Heavy chain | NI-105.6C5 | US20150344553 SEQ ID NO: 48 | 21501 |
| | | variable | | | |
| | | region | | | |
| TAU524 | Tau | Heavy chain | M-105.29G10 | US20150344553 SEQ ID NO: 50 | 21502 |
| | | variable | | | |
| | | region | | | |
| TAU525 | Tau | Heavy chain | NI-105.6L9 | US20150344553 SEQ ID NO: 52 | 21503 |
| | | variable | | | |
| | | region | | | |
| TAU526 | Tau | Heavy chain | NI-105.40E8 | US20150344553 SEQ ID NO: 54 | 21504 |
| | | variable | | | |
| | | region | | | |
| TAU527 | Tau | Heavy chain | NI-105.40E8 | US20150344553 SEQ ID NO: 220 | 21505 |
| | | variable | R104W | | |
| | | region | | | |
| TAU528 | Tau | Heavy chain | NI-105.48E5 | US20150344553 SEQ ID NO: 56 | 21506 |
| | | variable | | | |
| | | region | | | |
| TAU529 | Tau | Heavy chain | NI-105.6E3 | US20150344553 SEQ ID NO: 58 | 21507 |
| | | variable | | | |
| | | region | | | |
| TAU530 | Tau | Heavy chain | N1-105.22E1 | US20150344553 SEQ ID NO: 60 | 21508 |
| | | variable | | | |
| | | region | | | |
| TAU531 | Tau | Heavy chain | NI-105.26B12 | US20150344553 SEQ ID NO: 62 | 21509 |
| | | variable | | | |
| | | region | | | |
| TAU532 | Tau | Heavy chain | NI-105.12E12 | US20150344553 SEQ ID NO: 65 | 21510 |
| | | variable | | | |
| | | region | | | |
| TAU533 | Tau | Heavy chain | NI-105.60E7 | US20150344553 SEQ ID NO: 67 | 21511 |
| | | variable | | | |
| | | region | | | |
| TAU534 | Tau | Heavy chain | NI-105.14E2 | US20150344553 SEQ ID NO: 69 | 21512 |
| | | variable | | | |
| | | region | | | |
| TAU535 | Tau | Heavy chain | NI-105.39E2 | US20150344553 SEQ ID NO: 71 | 21513 |
| | | variable | | | |
| | | region | | | |
| TAU536 | Tau | Heavy chain | NI-105.19C6 | US20150344553 SEQ ID NO: 73 | 21514 |
| | | variable | | | |
| | | region | | | |
| TAU537 | Tau | Heavy chain | NI-105.9C4 | US20150344553 SEQ ID NO: 76 | 21515 |
| | | variable | | | |
| | | region | | | |
| TAU538 | Tau | Heavy chain | 19.3 | US20150320860 SEQ ID NO: 7 | 21516 |
| | | variable | | | |
| | | region | | | |
| TAU539 | Tau | Heavy chain | 3-66 | US20150320860 SEQ ID NO: 8 | 21517 |
| | | variable | | | |
| | | region | | | |
| TAU540 | Tau | Heavy chain | | US20150253341 SEQ ID NO: 37 | 21518 |
| | | variable | | | |
| | | region | | | |
| TAU541 | Tau | Heavy chain | NI-101.10 | US20150147343 SEQ ID NO: 4 | 21519 |
| | | variable | | | |
| | | region | | | |
| TAU542 | Tau | Heavy chain | NI-101.11 | US20150147343 SEQ ID NO: 6 | 21520 |
| | | variable | | | |
| | | region | | | |
| TAU543 | Tau | Heavy chain | NI-101.12 | US20150147343 SEQ ID NO: 10 | 21521 |
| | | variable | | | |
| | | region | | | |
| TAU544 | Tau | Heavy chain | NI-101.13; NI- | US20150147343 SEQ ID NO: 14, | 21522 |
| | | variable | 101.13A; NI- | 42, 43 | |
| | | region | 101.13B | | |
| TAU545 | Tau | Heavy chain | NI-101.12F6A | US20150147343 SEQ ID NO: 39 | 21523 |
| | | variable | | | |
| | | region | | | |
| TAU546 | Tau | Heavy chain | Ta1501 | US20150183854 SEQ ID NO: 18 | 21524 |
| | | variable | | | |
| | | region | | | |
| TAU547 | Tau | Heavy chain | Ta1502 | US20150183854 SEQ ID NO: 19 | 21525 |
| | | variable | | | |
| | | region | | | |
| TAU548 | Tau | Heavy chain | Ta1505 | US20150183854 SEQ ID NO: 20 | 21526 |
| | | variable | | | |
| | | region | | | |
| TAU549 | Tau | Heavy chain | Ta1506 | US20150183854 SEQ ID NO: 21 | 21527 |
| | | variable | | | |
| | | region | | | |
| TAU550 | Tau | Heavy chain | Ta1507 | US20150183854 SEQ ID NO: 22 | 21528 |
| | | variable | | | |
| | | region | | | |
| TAU551 | Tau | Heavy chain | Ta1508 | US20150183854 SEQ ID NO: 23 | 21529 |
| | | variable | | | |
| | | region | | | |
| TAU552 | Tau | Heavy chain | Ta1509 | US20150183854 SEQ ID NO: 24 | 21530 |
| | | variable | | | |
| | | region | | | |
| TAU553 | Tau | Heavy chain | | US20150050215 SEQ ID NO: 145 | 21531 |
| | | variable | | | |
| | | region | | | |
| TAU554 | Tau | Heavy chain | | US20150050215 SEQ ID NO: 147 | 21532 |
| | | variable | | | |
| | | region | | | |
| TAU555 | Tau | Heavy chain | | US20150050215 SEQ ID NO: 148 | 21533 |
| | | variable | | | |
| | | region | | | |
| TAU556 | Tau | Heavy chain | | U.S. Pat. No. 8,980,270 SEQ ID NO: 14 | 21534 |
| | | variable | | | |
| | | region | | | |
| TAU557 | Tau | Heavy chain | | U.S. Pat. No. 8,98,0270 SEQ ID NO: 16 | 21535 |
| | | variable | | | |
| | | region | | | |
| TAU558 | Tau | Heavy chain | | US20150183855 SEQ ID NO: 15; | 21536 |
| | | variable | | WO2016126993 SEQ ID NO: 15 | |
| | | region | | | |
| TAU559 | Tau | Heavy chain | CBTAU-7.1 | WO2015197823 SEQ ID NO: 87 | 21537 |
| | | variable | | | |
| | | region | | | |
| TAU560 | Tau | Heavy chain | CBTAU-8.1 | WO2015197823 SEQ ID NO: 91 | 21538 |
| | | variable | | | |
| | | region | | | |
| TAU561 | Tau | Heavy chain | CBTAU- 16.1 | WO2015197823 SEQ ID NO: 95 | 21539 |
| | | variable | | | |
| | | region | | | |
| TAU562 | Tau | Heavy chain | CBTAU-18.1 | WO2015197823 SEQ ID NO: 99 | 21540 |
| | | variable | | | |
| | | region | | | |
| TAU563 | Tau | Heavy chain | CBTAU-20.1 | WO2015197823 SEQ ID NO: 103 | 21541 |
| | | variable | | | |
| | | region | | | |
| TAU564 | Tau | Heavy chain | CBTAU-22.1 | WO2015197823 SEQ ID NO: 107 | 21542 |
| | | variable | | | |
| | | region | | | |
| TAU565 | Tau | Heavy chain | CBTAU-24.1 | WO2015197823 SEQ ID NO: 111 | 21543 |
| | | variable | | | |
| | | region | | | |
| TAU566 | Tau | Heavy chain | CBTAU-27.1 | WO2015197823 SEQ ID NO: 115 | 21544 |
| | | variable | | | |
| | | region | | | |
| TAU567 | Tau | Heavy chain | CBTAU 28.1 | WO2015197823 SEQ ID NO: 119 | 21545 |
| | | variable | | | |
| | | region | | | |
| TAU568 | Tau | Heavy chain | CBTAU -41.1 | WO2015197823 SEQ ID NO: 123 | 21546 |
| | | variable | | | |
| | | region | | | |
| TAU569 | Tau | Heavy chain | CBTAU -41.2 | WO2015197823 SEQ ID NO: 127 | 21547 |
| | | variable | | | |
| | | region | | | |
| TAU570 | Tau | Heavy chain | CBTAU -42.1 | WO2015197823 SEQ ID NO: 131 | 21548 |
| | | variable | | | |
| | | region | | | |
| TAU571 | Tau | Heavy chain | CBTAU 43.1 | WO2015197823 SEQ ID NO: 135 | 21549 |
| | | variable | | | |
| | | region | | | |
| TAU572 | Tau | Heavy chain | CBTAU 44.1 | WO2015197823 SEQ ID NO: 139 | 21550 |
| | | variable | | | |
| | | region | | | |
| TAU573 | Tau | Heavy chain | CBTAU 45.1 | WO2015197823 SEQ ID NO: 143 | 21551 |
| | | variable | | | |
| | | region | | | |
| TAU574 | Tau | Heavy chain | CBTAU 46.1 | WO2015197823 SEQ ID NO: 147 | 21552 |
| | | variable | | | |
| | | region | | | |
| TAU575 | Tau | Heavy chain | CBTAU 47.1 | WO2015197823 SEQ ID NO: 151 | 21553 |
| | | variable | | | |
| | | region | | | |
| TAU576 | Tau | Heavy chain | CBTAU 47.2 | WO2015197823 SEQ ID NO: 155 | 21554 |
| | | variable | | | |
| | | region | | | |
| TAU577 | Tau | Heavy chain | CBTAU 49.1 | WO2015197823 SEQ ID NO: 159 | 21555 |
| | | variable | | | |
| | | region | | | |
| TAU578 | Tau | Heavy chain | Native 7.1 | WO2015197823 SEQ ID NO: 257 | 21556 |
| | | variable | | | |
| | | region | | | |
| TAU579 | Tau | Heavy chain | Native 8.1 | WO2015197823 SEQ ID NO: 261 | 21557 |
| | | variable | | | |
| | | region | | | |
| TAU580 | Tau | Heavy chain | Native 16.1 | WO2015197823 SEQ ID NO: 265 | 21558 |
| | | variable | | | |
| | | region | | | |
| TAU581 | Tau | Heavy chain | Native 18.1 | WO2015197823 SEQ ID NO: 269 | 21559 |
| | | variable | | | |
| | | region | | | |
| TAU582 | Tau | Heavy chain | Native 20.1 | WO2015197823 SEQ ID NO: 272 | 21560 |
| | | variable | | | |
| | | region | | | |
| TAU583 | Tau | Heavy chain | Native 22.1 | WO2015197823 SEQ ID NO: 275 | 21561 |
| | | variable | | | |
| | | region | | | |
| TAU584 | Tau | Heavy chain | Native 24.1 | WO2015197823 SEQ ID NO: 279 | 21562 |
| | | variable | | | |
| | | region | | | |
| TAU585 | Tau | Heavy chain | Native 27.1 | WO2015197823 SEQ ID NO: 282 | 21563 |
| | | variable | | | |
| | | region | | | |
| TAU586 | Tau | Heavy chain | Native 28.1 | WO2015197823 SEQ ID NO: 284 | 21564 |
| | | variable | | | |
| | | region | | | |
| TAU587 | Tau | Heavy chain | Native 41.1; | WO2015197823 SEQ ID NO: 287, | 21565 |
| | | variable | Native 41.2 | 289 | |
| | | region | | | |
| TAU588 | Tau | Heavy chain | Native 42.1 | WO2015197823 SEQ ID NO: 292 | 21566 |
| | | variable | | | |
| | | region | | | |
| TAU589 | Tau | Heavy chain | Native 43.1 | WO2015197823 SEQ ID NO: 295 | 21567 |
| | | variable | | | |
| | | region | | | |
| TAU590 | Tau | Heavy chain | Native 44.1 | WO2015197823 SEQ ID NO: 298 | 21568 |
| | | variable | | | |
| | | region | | | |
| TAU591 | Tau | Heavy chain | Native 45.1 | WO2015197823 SEQ ID NO: 302 | 21569 |
| | | variable | | | |
| | | region | | | |
| TAU592 | Tau | Heavy chain | Native 46.1 | WO2015197823 SEQ ID NO: 306 | 21570 |
| | | variable | | | |
| | | region | | | |
| TAU593 | Tau | Heavy chain | Native 47.1 | WO2015197823 SEQ ID NO: 309 | 21571 |
| | | variable | | | |
| | | region | | | |
| TAU594 | Tau | Heavy chain | Native 47.2 | WO2015197823 SEQ ID NO: 311 | 21572 |
| | | variable | | | |
| | | region | | | |
| TAU595 | Tau | Heavy chain | Native 49.1 | WO2015197823 SEQ ID NO: 313 | 21573 |
| | | variable | | | |
| | | region | | | |
| TAU596 | Tau | Heavy chain | 6B2G12; | WO2016007414 SEQ ID NO: 9 | 21574 |
| | | variable | scFv235 | and 11 | |
| | | region | | | |
| TAU597 | Tau | Heavy chain | | WO2015120364 SEQ ID NO: 30 | 21575 |
| | | variable | | | |
| | | region | | | |
| TAU598 | Tau | Heavy chain | | WO2015120364 SEQ ID NO: 42 | 21576 |
| | | variable | | | |
| | | region | | | |
| TAU599 | Tau | Heavy chain | pT231/pS235_1; | WO2014016737 SEQ ID NO: 15 | 21577 |
| | | variable | pT231/pS235_2 | and 17 | |
| | | region | | | |
| TAU600 | Tau | Heavy chain | pT212/pS214_1 | WO2014016737 SEQ ID NO: 19 | 21578 |
| | | variable | | | |
| | | region | | | |
| TAU601 | Tau | Heavy chain | pT212/pS214_2 | WO2014016737 SEQ ID NO: 21 | 21579 |
| | | variable | | | |
| | | region | | | |
| TAU602 | Tau | Heavy chain | pS396/pS404_1 | WO2014016737 SEQ ID NO: 23 | 21580 |
| | | variable | | | |
| | | region | | | |
| TAU603 | Tau | Heavy chain | pS396/pS404_2 | WO2014016737 SEQ ID NO: 25 | 21581 |
| | | variable | | | |
| | | region | | | |
| TAU604 | Tau | Heavy chain | 2H9 | WO2014096321 SEQ ID NO: 11 | 21582 |
| | | variable | | | |
| | | region | | | |
| TAU605 | Tau | Heavy chain | | WO2015122922 SEQ ID NO: 16 | 21583 |
| | | variable | | and 24 | |
| | | region | | | |
| TAU606 | Tau | Heavy chain | | WO2015122922 SEQ ID NO: 32 | 21584 |
| | | variable | | | |
| | | region | | | |
| TAU607 | Tau | Heavy chain | | WO2015122922 SEQ ID NO: 40 | 21585 |
| | | variable | | | |
| | | region | | | |
| TAU608 | Tau | Heavy chain | | WO2015122922 SEQ ID NO: 48 | 21586 |
| | | variable | | | |
| | | region | | | |
| TAU609 | Tau | Heavy chain | | WO2015122922 SEQ ID NO: 56 | 21587 |
| | | variable | | | |
| | | region | | | |
| TAU610 | Tau | Heavy chain | | WO2015122922 SEQ ID NO: 64 | 21588 |
| | | variable | | | |
| | | region | | | |
| TAU611 | Tau | Heavy chain | | WO2015122922 SEQ ID NO: 72 | 21589 |
| | | variable | | | |
| | | region | | | |
| TAU612 | Tau | Heavy chain | | US20150320860 SEQ ID NO: 34 | 21590 |
| | | variable | | | |
| | | region fused | | | |
| | | with a human | | | |
| | | IgG2 heavy | | | |
| | | chain | | | |
| | | constant | | | |
| | | region | | | |
| TAU613 | Tau | Heavy chain | NI-105.17C1 | US20150344553 SEQ ID NO: 44 | 21591 |
| | | variable | | | |
| | | region, before | | | |
| | | germlining | | | |
| TAU614 | Tau | Heavy chain | NI-105.6C5 | US20150344553 SEQ ID NO: 47 | 21592 |
| | | variable | | | |
| | | region, before | | | |
| | | germlining | | | |
| TAU615 | Tau | Heavy chain | NI-105.26B12 | US20150344553 SEQ ID NO: 63 | 21593 |
| | | variable | | | |
| | | region, before | | | |
| | | germlining | | | |
| TAU616 | Tau | Heavy chain | NI-105.9C4 | US20150344553 SEQ ID NO: 75 | 21594 |
| | | variable | | | |
| | | region, before | | | |
| | | germlining | | | |
| TAU617 | Tau | Heavy chain | variant 1-VH32 | US20150175685 SEQ ID NO: 19; | 21595 |
| | | variable | | WO2015197735 SEQ ID NO: 19 | |
| | | region, | | | |
| | | humanized | | | |
| TAU618 | Tau | Heavy chain | variant 2-VH20 | US20150175685 SEQ ID NO: 20; | 21596 |
| | | variable | | WO2015197735 SEQ ID NO: 20 | |
| | | region. | | | |
| | | humanized | | | |
| TAU619 | Tau | Heavy chain | IPN002 VH | U.S. Pat. No. 8,980,270 SEQ ID NO: 36 | 21597 |
| | | variable | variant 1 | | |
| | | region, | | | |
| | | humanized | | | |
| TAU620 | Tau | Heavy chain | IPN002 VH | U.S. Pat. No. 8,980,270 SEQ ID NO: 37 | 21598 |
| | | variable | variant 2 | | |
| | | region, | | | |
| | | humanized | | | |
| TAU621 | Tau | Heavy chain | IPN002 VH | U.S. Pat. No. 8,980,270 SEQ ID NO: 38 | 21599 |
| | | variable | variant 3 | | |
| | | region, | | | |
| | | humanized | | | |
| TAU622 | Tau | Heavy chain | IPN002 VHI | U.S. Pat. No. 8,980,270 SEQ ID NO: 39 | 21600 |
| | | variable | variant 4 | | |
| | | region, | | | |
| | | humanized | | | |
| TAU623 | Tau | Heavy chain, | BACO2002.1 | US20160031976 SEQ ID NO: 14 | 21601 |
| | | human Ig | | | |
| TAU624 | Tau | Heavy chain, | | US20160031976 SEQ ID NO: 29 | 21602 |
| | | human IgG1 | | | |
| | | constant | | | |
| | | region | | | |
| TAU625 | Tau | Heavy chain, | TAM_1, | US20160024193 SEQ ID NO: 87 | 21603 |
| | | IgG1 | TAM_2, | | |
| | | | TAM_3, | | |
| | | | TAM_4, | | |
| | | | TAM_5, | | |
| | | | TAM_6, | | |
| | | | TAM_7, | | |
| | | | TAM_8, | | |
| | | | TAM_9, | | |
| | | | TAM_10, | | |
| | | | TAM_11, | | |
| | | | TAM_12, | | |
| | | | TAM_13, | | |
| | | | TAM_14, | | |
| | | | TAM_15, | | |
| | | | TAM_16, | | |
| | | | TAM_17, | | |
| | | | TAM_18, | | |
| | | | TAM_19, | | |
| | | | TAM_20, | | |
| | | | TAM_21, | | |
| | | | TAM_22, | | |
| | | | TAM_23 | | |
| TAU626 | Tau | Heavy chain, | TAM_1, | US20160024193 SEQ ID NO: 88 | 21604 |
| | | IgG1 N297G | TAM_2, | | |
| | | | TAM_3, | | |
| | | | TAM_4, | | |
| | | | TAM_5, | | |
| | | | TAM_6, | | |
| | | | TAM_7, | | |
| | | | TAM_8, | | |
| | | | TAM_9, | | |
| | | | TAM_10, | | |
| | | | TAM_11, | | |
| | | | TAM_12. | | |
| | | | TAM_13, | | |
| | | | TAM_14, | | |
| | | | TAM_15, | | |
| | | | TAM_16, | | |
| | | | TAM_17, | | |
| | | | TAM_18, | | |
| | | | TAM_19, | | |
| | | | TAM_20, | | |
| | | | TAM_21, | | |
| | | | TAM_22, | | |
| | | | TAM_23 | | |
| TAU627 | Tau | Heavy chain, | TAM_1, | US20160024193 SEQ ID NO: 86 | 21605 |
| | | IgG4 isotypes | TAM_2, | | |
| | | | TAM_3, | | |
| | | | TAM_4, | | |
| | | | TAM_5, | | |
| | | | TAM_6, | | |
| | | | TAM_7, | | |
| | | | TAM_8, | | |
| | | | TAM_9, | | |
| | | | TAM_10, | | |
| | | | TAM_11, | | |
| | | | TAM_12, | | |
| | | | TAM_13, | | |
| | | | TAM_14, | | |
| | | | TAM_15, | | |
| | | | TAM_16, | | |
| | | | TAM_17, | | |
| | | | TAM_18, | | |
| | | | TAM_19, | | |
| | | | TAM_20, | | |
| | | | TAM_21, | | |
| | | | TAM_22, | | |
| | | | TAM_23 | | |
| TAU628 | Tau | Heavy chain, | | US20160031976 SEQ ID NO: 15 | 21606 |
| | | mature | | | |
| TAU629 | Tau | heavy-chain | Tau-A2-SH | WO2015114538 SEQ ID NO: 14 | 21607 |
| | | antibody; | | | |
| | | camelid | | | |
| TAU630 | Tau | heavy-chain | TauA2var-SH | WO2015114538 SEQ ID NO: 17 | 21608 |
| | | antibody; | | | |
| | | Camelid | | | |
| TAU631 | Tau | heavy-chain | Tau-A2 variant | WO2015114538 SEQ ID NO: 15 | 21609 |
| | | antibody; | | | |
| | | Camelid | | | |
| TAU632 | Tau | heavy-chain | Tau-A2 variant | WO2015114538 SEQ ID NO: 16 | 21610 |
| | | antibody; | | | |
| | | Camelid | | | |
| TAU633 | Tau | Light chain | cDC8E8 VK | US20150050215 SEQ ID NO: | 21611 |
| | | | | 141; WO2016079597 SEQ ID NO: | |
| | | | | 10 | |
| TAU634 | Tau | Light chain | RKA | WO2016079597 SEQ ID NO: 57 | 21612 |
| TAU635 | Tau | Light chain | cDC8E8 | WO2016079597 SEQ ID NO: 59 | 21613 |
| TAU636 | Tau | Light chain | OptiDC8E8 | WO2016079597 SEQ ID NO: 95 | 21614 |
| TAU637 | Tau | Light chain | RKA | WO2016079597 SEQ ID NO: 109 | 21615 |
| TAU638 | Tau | Light chain | RKB | WO2016079597 SEQ ID NO: 110 | 21616 |
| TAU639 | Tau | Light chain | RKA | WO2016079597 SEQ ID NO: 141 | 21617 |
| TAU640 | Tau | Light chain | RKB | WO2016079597 SEQ ID NO: 142 | 21618 |
| TAU641 | Tau | Light chain | cDC8E8 | WO2016079597 SEQ ID NO: 143 | 21619 |
| TAU642 | Tau | Light chain | | U.S. Pat. No. 8,697,076 SEQ ID NO: 14 | 21620 |
| TAU643 | Tau | Light chain | 5202.4 | US20160024193 SEQ ID NO: 61 | 21621 |
| TAU644 | Tau | Light chain | TAM_1 | US20160024193 SEQ ID NO: 64 | 21622 |
| TAU645 | Tau | Light chain | TAM_2 | US20160024193 SEQ ID NO: 65 | 21623 |
| TAU646 | Tau | Light chain | TAM_3 | US20160024193 SEQ ID NO: 66 | 21624 |
| TAU647 | Tau | Light chain | TAM_4 | US20160024193 SEQ ID NO: 67 | 21625 |
| TAU648 | Tau | Light chain | TAM_5 | US20160024193 SEQ ID NO: 68 | 21626 |
| TAU649 | Tau | Light chain | TAM_6 | US20160024193 SEQ ID NO: 69 | 21627 |
| TAU650 | Tau | Light chain | TAM_7 | US20160024193 SEQ ID NO: 70 | 21628 |
| TAU651 | Tau | Light chain | TAM_8 | US20160024193 SEQ ID NO: 71 | 21629 |
| TAU652 | Tau | Light chain | TAM_9 | US20160024193 SEQ ID NO: 72 | 21630 |
| TAU653 | Tau | Light chain | TAM_10 | US20160024193 SEQ ID NO: 73 | 21631 |
| TAU654 | Tau | Light chain | TAM_11 | US20160024193 SEQ ID NO: 74 | 21632 |
| TAU655 | Tau | Light chain | TAM_12 | US20160024193 SEQ ID NO: 75 | 21633 |
| TAU656 | Tau | Light chain | TAM_13 | US20160024193 SEQ ID NO: 76 | 21634 |
| TAU657 | Tau | Light chain | TAM_14 | US20160024193 SEQ ID NO: 77 | 21635 |
| TAU658 | Tau | Light chain | TAM_15 | US20160024193 SEQ ID NO: 78 | 21636 |
| TAU659 | Tau | Light chain | TAM_16 | US20160024193 SEQ ID NO: 79 | 21637 |
| TAU660 | Tau | Light chain | TAM_17 | US20160024193 SEQ ID NO: 80 | 21638 |
| TAU661 | Tau | Light chain | TAM_18 | US20160024193 SEQ ID NO: 81 | 21639 |
| TAU662 | Tau | Light chain | TAM_19 | US20160024193 SEQ ID NO: 82 | 21640 |
| TAU663 | Tau | Light chain | TAM_20; | US20160024193 SEQ ID NO: 83 | 21641 |
| | | | TAM_22 | and 85 | |
| TAU664 | Tau | Light chain | TAM_21 | US20160024193 SEQ ID NO: 84 | 21642 |
| TAU665 | Tau | Light chain | | US20160031977 SEQ ID NO: 23 | 21643 |
| TAU666 | Tau | Light chain | | US20160031977 SEQ ID NO: 25 | 21644 |
| TAU667 | Tau | Light chain | | US20160031977 SEQ ID NO: 27 | 21645 |
| TAU668 | Tau | Light chain | | US20160031977 SEQ ID NO: 28 | 21646 |
| TAU669 | Tau | Light chain | | US20150050215 SEQ ID NO: 139 | 21647 |
| TAU670 | Tau | Light chain | | US20150050215 SEQ ID NO: 143 | 21648 |
| TAU671 | Tau | Light Chain | pT231/pS235 | WO2014016737 SEQ ID NO: 71 | 21649 |
| TAU672 | Tau | Light chain | RKB | WO2016079597 SEQ ID NO: 58 | 21650 |
| TAU673 | Tau | Light chain | cDC8E8 | WO2016079597 SEQ ID NO: 93 | 21651 |
| TAU674 | Tau | light chain | ch40E8 | US20150344553 SEQ ID NO: 209 | 21652 |
| | | (lambda) | | | |
| TAU675 | Tau | light chain | ch6E3 | US20150344553 SEQ ID NO: 211 | 21653 |
| | | (mouse | | | |
| | | kappa) | | | |
| TAU676 | Tau | light chain | ch17C1 | US20150344553 SEQ ID NO: 204 | 21654 |
| | | (mouse | | | |
| | | lambda) | | | |
| TAU677 | Tau | light chain | ch6C5 | US20150344553 SEQ ID NO: 206 | 21655 |
| | | (mouse | | | |
| | | lambda) | | | |
| TAU678 | Tau | light chain | ch17C1(N31Q) | US20150344553 SEQ ID NO: 212 | 21656 |
| | | (mouse | | | |
| | | lambda) | | | |
| TAU679 | Tau | light chain | | WO2016079597 SEQ ID NO: 170; | 21657 |
| | | constant | | WO2015197823 SEQ ID NO: 84; | |
| | | region | | US20150320860 SEQ ID NO: 36; | |
| | | | | WO2015197735 SEQ ID NO: 59; | |
| | | | | U.S. Pat. No. 9,290,567 SEQ ID NO: 11 | |
| TAU680 | Tau | light chain | | WO2016079597 SEQ ID NO: 171; | 21658 |
| | | constant | | US20160031976 SEQ ID NO: 32 | |
| | | region | | | |
| TAU681 | Tau | Light chain | human NI- | US20150344553 SEQ ID NO: 219 | 21659 |
| | | lambda | 105.40E8 light | | |
| | | | chain | | |
| TAU682 | Tau | Light chain | ch17C1(N31Q, | US20150344553 SEQ ID NO: 217 | 21660 |
| | | lambda | 148V) | | |
| | | mouse | | | |
| TAU683 | Tau | Light chain | ch4E4 | US20150344553 SEQ ID NO: 21; | 21661 |
| | | mature | | U.S. Pat. No. 8,940,272 SEQ ID NO: 21 | |
| | | (mouse | | | |
| | | lambda) | | | |
| TAU684 | Tau | Light chain | NI-105.4A3-VL | US20150344553 SEQ ID NO: 19; | 21662 |
| | | variable | | U.S. Pat. No. 8,940,272 SEQ ID NO: 19 | |
| TAU685 | Tau | Light chain | | US20150344553 SEQ ID NO: 15 | 21663 |
| | | variable | | | |
| TAU686 | Tau | Light chain | NI-105.4E4-VL; | US20150344553 SEQ ID NO: 11, | 21664 |
| | | variable | NI-105.24B2-VL | 15 | |
| TAU687 | Tau | Light chain | | US20150307600 SEQ ID NO: 36 | 21665 |
| | | variable | | | |
| TAU688 | Tau | Light chain | | US20150307600 SEQ ID NO: 38 | 21666 |
| | | variable | | | |
| TAU689 | Tau | Light chain | RKA | WO2016079597 SEQ ID NO: 26 | 21667 |
| | | variable | | | |
| | | region | | | |
| TAU690 | Tau | Light chain | RKB | WO2016079597 SEQ ID NO: 27 | 21668 |
| | | variable | | | |
| | | region | | | |
| TAU691 | Tau | Light chain | DC8E8 | WO2016079597 SEQ ID NO: 91 | 21669 |
| | | variable | | | |
| | | region | | | |
| TAU692 | Tau | Light chain | | U.S. Pat. No. 8,940,272 SEQ ID NO: 15 | 21670 |
| | | variable | | | |
| | | region | | | |
| TAU693 | Tau | Light chain | | U.S. Pat. No. 8,697,076 SEQ ID NO: 8 | 21671 |
| | | variable | | | |
| | | region | | | |
| TAU694 | Tau | Light chain | | US20160024193 SEQ ID NO: 36 | 21672 |
| | | variable | | | |
| | | region | | | |
| TAU695 | Tau | Light chain | | US20160024193 SEQ ID NO: 37 | 21673 |
| | | variable | | | |
| | | region | | | |
| TAU696 | Tau | Light chain | | US20160024193 SEQ ID NO: 38 | 21674 |
| | | variable | | | |
| | | region | | | |
| TAU697 | Tau | Light chain | | US20160024193 SEQ ID NO: 39 | 21675 |
| | | variable | | | |
| | | region | | | |
| TAU698 | Tau | Light chain | | US20160024193 SEQ ID NO: 40 | 21676 |
| | | variable | | | |
| | | region | | | |
| TAU699 | Tau | Light chain | | US20160024193 SEQ ID NO: 41 | 21677 |
| | | variable | | | |
| | | region | | | |
| TAU700 | Tau | Light chain | | US20160024193 SEQ ID NO: 42 | 21678 |
| | | variable | | | |
| | | region | | | |
| TAU701 | Tau | Light chain | | US20160024193 SEQ ID NO: 43 | 21679 |
| | | variable | | | |
| | | region | | | |
| TAU702 | Tau | Light chain | | US20160024193 SEQ ID NO: 44 | 21680 |
| | | variable | | | |
| | | region | | | |
| TAU703 | Tau | Light chain | | US20160024193 SEQ ID NO: 45 | 21681 |
| | | variable | | | |
| | | region | | | |
| TAU704 | Tau | Light chain | | US20160024193 SEQ ID NO: 46 | 21682 |
| | | variable | | | |
| | | region | | | |
| TAU705 | Tau | Light chain | | US20160024193 SEQ ID NO: 47 | 21683 |
| | | variable | | | |
| | | region | | | |
| TAU706 | Tau | Light chain | | US20160024193 SEQ ID NO: 48 | 21684 |
| | | variable | | | |
| | | region | | | |
| TAU707 | Tau | Light chain | | US20160024193 SEQ ID NO: 49 | 21685 |
| | | variable | | | |
| | | region | | | |
| TAU708 | Tau | Light chain | | US20160024193 SEQ ID NO: 50 | 21686 |
| | | variable | | | |
| | | region | | | |
| TAU709 | Tau | Light chain | | US20160024193 SEQ ID NO: 51 | 21687 |
| | | variable | | | |
| | | region | | | |
| TAU710 | Tau | Light chain | | US20160024193 SEQ ID NO: 52 | 21688 |
| | | variable | | | |
| | | region | | | |
| TAU711 | Tau | Light chain | | US20160024193 SEQ ID NO: 53 | 21689 |
| | | variable | | | |
| | | region | | | |
| TAU712 | Tau | Light chain | | US20160024193 SEQ ID NO: 54 | 21690 |
| | | variable | | | |
| | | region | | | |
| TAU713 | Tau | Light chain | | US20160024193 SEQ ID NO: 55 | 21691 |
| | | variable | | and 57 | |
| | | region | | | |
| TAU714 | Tau | Light chain | | US20160024193 SEQ ID NO: 56 | 21692 |
| | | variable | | | |
| | | region | | | |
| TAU715 | Tau | Light chain | 5202.4 | US20160024193 SEQ ID NO: 60 | 21693 |
| | | variable | | | |
| | | region | | | |
| TAU716 | Tau | Light chain | NI-105.17C1 | US20150344553 SEQ ID NO: 46 | 21694 |
| | | variable | | | |
| | | region | | | |
| TAU717 | Tau | Light chain | NI-105.17C1 | US20150344553 SEQ ID NO: 221 | 21695 |
| | | variable | N31Q | | |
| | | region | | | |
| TAU718 | Tau | Light chain | NI-105,17C1 | US20150344553 SEQ ID NO: 222 | 21696 |
| | | variable | N31Q, 148V | | |
| | | region | | | |
| TAU719 | Tau | Light chain | NI-105.6C5 | US20150344553 SEQ ID NO: 49 | 21697 |
| | | variable | | | |
| | | region | | | |
| TAU720 | Tau | Light chain | M-105.29G10 | US20150344553 SEQ ID NO: 51 | 21698 |
| | | variable | | | |
| | | region | | | |
| TAU721 | Tau | Light chain | NI-105.6L9 | US20150344553 SEQ ID NO: 53 | 21699 |
| | | variable | | | |
| | | region | | | |
| TAU722 | Tau | Light chain | NI-105.40E8 | US20150344553 SEQ ID NO: 55 | 21700 |
| | | variable | | | |
| | | region | | | |
| TAU723 | Tau | Light chain | NI-105,48E5 | US20150344553 SEQ ID NO: 57 | 21701 |
| | | variable | | | |
| | | region | | | |
| TAU724 | Tau | Light chain | NI-105.6E3 | US20150344553 SEQ ID NO: 59 | 21702 |
| | | variable | | | |
| | | region | | | |
| TAU725 | Tau | Light chain | NI-105.22E1 | US20150344553 SEQ ID NO: 61 | 21703 |
| | | variable | | | |
| | | region | | | |
| TAU726 | Tau | Light chain | NI-105.26B13 | US20150344553 SEQ ID NO: 64 | 21704 |
| | | variable | | | |
| | | region | | | |
| TAU727 | Tau | Light chain | NI-105.12E12 | US20150344553 SEQ ID NO: 66 | 21705 |
| | | variable | | | |
| | | region | | | |
| TAU728 | Tau | Light chain | NI-105,60E7 | US20150344553 SEQ ID NO: 68 | 21706 |
| | | variable | | | |
| | | region | | | |
| TAU729 | Tau | Light chain | NI-105.14E2 | US20150344553 SEQ ID NO: 70 | 21707 |
| | | variable | | | |
| | | region | | | |
| TAU730 | Tau | Light chain | NI-105.39E2 | US20150344553 SEQ ID NO: 72 | 21708 |
| | | variable | | | |
| | | region | | | |
| TAU731 | Tau | Light chain | NI-105.19C6 | US20150344553 SEQ ID NO: 74 | 21709 |
| | | variable | | | |
| | | region | | | |
| TAU732 | Tau | Light chain | N1-105.9C4 | US20150344553 SEQ ID NO: 78 | 21710 |
| | | variable | | | |
| | | region | | | |
| TAU733 | Tau | Light chain | 19.3 | US20150320860 SEQ ID NO: 9 | 21711 |
| | | variable | | | |
| | | region | | | |
| TAU734 | Tau | Light chain | 3-66 | US20150320860 SEQ ID NO: 10 | 21712 |
| | | variable | | | |
| | | region | | | |
| TAU735 | Tau | Light chain | h3B3 | US20150320860 SEQ ID NO: 25 | 21713 |
| | | variable | | | |
| | | region | | | |
| TAU736 | Tau | Light chain | 19.3 | US20150320860 SEQ ID NO: 26 | 21714 |
| | | variable | | | |
| | | region | | | |
| TAU737 | Tau | Light chain | 17.1 | US20150320860 SEQ ID NO: 27 | 21715 |
| | | variable | | | |
| | | region | | | |
| TAU738 | Tau | Light chain | 14.2 | US20150320860 SEQ ID NO: 28 | 21716 |
| | | variable | | | |
| | | region | | | |
| TAU739 | Tau | Light chain | 13.1 | US20150320860 SEQ ID NO: 29 | 21717 |
| | | variable | | | |
| | | region | | | |
| TAU740 | Tau | Light chain | 7.2 | US20150320860 SEQ ID NO: 30 | 21718 |
| | | variable | | | |
| | | region | | | |
| TAU741 | Tau | Light chain | 9.2 | US20150320860 SEQ ID NO: 31 | 21719 |
| | | variable | | | |
| | | region | | | |
| TAU742 | Tau | Light chain | 11.4 | US20150320860 SEQ ID NO: 32 | 21720 |
| | | variable | | | |
| | | region | | | |
| TAU743 | Tau | Light chain | | US20150253341 SEQ ID NO: 39 | 21721 |
| | | variable | | | |
| | | region | | | |
| TAU744 | Tau | Light chain | NI-101.10; NI- | US20150147343 SEQ ID NO: 8 | 21722 |
| | | variable | 101.11 | | |
| | | region | | | |
| TAU745 | Tau | Light chain | NI-101.12 | US20150147343 SEQ ID NO: 12 | 21723 |
| | | variable | | | |
| | | region | | | |
| TAU746 | Tau | Light chain | NI-101.13 | US20150147343 SEQ ID NO: 16 | 21724 |
| | | variable | | | |
| | | region | | | |
| TAU747 | Tau | Light chain | NI-101,12F6A | US20150147343 SEQ ID NO: 41 | 21725 |
| | | variable | | | |
| | | region | | | |
| TAU748 | Tau | Light chain | NI-101.13A | US20150147343 SEQ ID NO: 44 | 21726 |
| | | variable | | | |
| | | region | | | |
| TAU749 | Tau | Light chain | NI-101.13B | US20150147343 SEQ ID NO: 45 | 21727 |
| | | variable | | | |
| | | region | | | |
| TAU750 | Tau | Light chain | Tal501 | US20150183854 SEQ ID NO: 25 | 21728 |
| | | variable | | | |
| | | region | | | |
| TAU751 | Tau | Light chain | Tal502; Ta1505 | US20150183854 SEQ ID NO: 26 | 21729 |
| | | variable | | | |
| | | region | | | |
| TAU752 | Tau | Light chain | Ta1506 | US20150183854 SEQ ID NO: 27 | 21730 |
| | | variable | | | |
| | | region | | | |
| TAU753 | Tau | Light chain | Ta1507 | US20150183854 SEQ ID NO: 28 | 21731 |
| | | variable | | | |
| | | region | | | |
| TAU754 | Tau | Light chain | Tal508 | US20150183854 SEQ ID NO: 29 | 21732 |
| | | variable | | | |
| | | region | | | |
| TAU755 | Tau | Light chain | Tal509 | US20150183854 SEQ ID NO: 30 | 21733 |
| | | variable | | | |
| | | region | | | |
| TAU756 | Tau | Light chain | | US20150050215 SEQ ID NO: 150 | 21734 |
| | | variable | | | |
| | | region | | | |
| TAU757 | Tau | Light chain | | US20150050215 SEQ ID NO: 152 | 21735 |
| | | variable | | | |
| | | region | | | |
| TAU758 | Tau | Light chain | | US20150050215 SEQ ID NO: 153 | 21736 |
| | | variable | | | |
| | | region | | | |
| TAU759 | Tau | Light chain | | U.S. Pat. No. 8,980,270 SEQ ID NO: 13 | 21737 |
| | | variable | | | |
| | | region | | | |
| TAU760 | Tau | Light chain | | U.S. Pat. No. 8,980,270 SEQ ID NO: 15 | 21738 |
| | | variable | | | |
| | | region | | | |
| TAU761 | Tau | Light chain | CBTAU-7.1 | WO2015197823 SEQ ID NO: 88 | 21739 |
| | | variable | | | |
| | | region | | | |
| TAU762 | Tau | Light chain | CBTAU-8.1 | WO2015197823 SEQ ID NO: 92 | 21740 |
| | | variable | | | |
| | | region | | | |
| TAU763 | Tau | Light chain | CBTAU- 16.1 | WO2015197823 SEQ ID NO: 96 | 21741 |
| | | variable | | | |
| | | region | | | |
| TAU764 | Tau | Light chain | CBTAU- 18.1 | WO2015197823 SEQ ID NO: 100 | 21742 |
| | | variable | | | |
| | | region | | | |
| TAU765 | Tau | Light chain | CBTAU-20.1 | WO2015197823 SEQ ID NO: 104 | 21743 |
| | | variable | | | |
| | | region | | | |
| TAU766 | Tau | Light chain | CBTAU-22.1 | WO2015197823 SEQ ID NO: 108 | 21744 |
| | | variable | | | |
| | | region | | | |
| TAU767 | Tau | Light chain | CBTAU-24.1 | WO2015197823 SEQ ID NO: 112 | 21745 |
| | | variable | | | |
| | | region | | | |
| TAU768 | Tau | Light chain | CBTAU-27.1 | WO2015197823 SEQ ID NO: 116 | 21746 |
| | | variable | | | |
| | | region | | | |
| TAU769 | Tau | Light chain | CBTAU 28.1 | WO2015197823 SEQ ID NO: 120 | 21747 |
| | | variable | | | |
| | | region | | | |
| TAU770 | Tau | Light chain | CBTAU ~41.1 | WO2015197823 SEQ ID NO: 124 | 21748 |
| | | variable | | | |
| | | region | | | |
| TAU771 | Tau | Light chain | CBTAU -41.2 | WO2015197823 SEQ ID NO: 128 | 21749 |
| | | variable | | | |
| | | region | | | |
| TAU772 | Tau | Light chain | CBTAU -42.1 | WO2015197823 SEQ ID NO: 132 | 21750 |
| | | variable | | | |
| | | region | | | |
| TAU773 | Tau | Light chain | CBTAU 43.1 | WO2015197823 SEQ ID NO: 136 | 21751 |
| | | variable | | | |
| | | region | | | |
| TAU774 | Tau | Light chain | CBTAU 44.1 | WO2015197823 SEQ ID NO: 140 | 21752 |
| | | variable | | | |
| | | region | | | |
| TAU775 | Tau | Light chain | CBTAU 45.1 | WO2015197823 SEQ ID NO: 144 | 21753 |
| | | variable | | | |
| | | region | | | |
| TAU776 | Tau | Light chain | CBTAU 46.1 | WO2015197823 SEQ ID NO: 148 | 21754 |
| | | variable | | | |
| | | region | | | |
| TAU777 | Tau | Light chain | CBTAU 47.1 | WO2015197823 SEQ ID NO: 152 | 21755 |
| | | variable | | | |
| | | region | | | |
| TAU778 | Tau | Light chain | CBTAU 47.2 | WO2015197823 SEQ ID NO: 156 | 21756 |
| | | variable | | | |
| | | region | | | |
| TAU779 | Tau | Light chain | CBTAU 49.1 | WO2015197823 SEQ ID NO: 160 | 21757 |
| | | variable | | | |
| | | region | | | |
| TAU780 | Tau | Light chain | Native 7.1 | WO2015197823 SEQ ID NO: 259 | 21758 |
| | | variable | | | |
| | | region | | | |
| TAU781 | Tau | Light chain | Native 8.1 | WO2015197823 SEQ ID NO: 263 | 21759 |
| | | variable | | | |
| | | region | | | |
| TAU782 | Tau | Light chain | Native 16.1 | WO2015197823 SEQ ID NO: 267 | 21760 |
| | | variable | | | |
| | | region | | | |
| TAU783 | Tau | Light chain | Native 18.1 | WO2015197823 SEQ ID NO: 270 | 21761 |
| | | variable | | | |
| | | region | | | |
| TAU784 | Tau | Light chain | Native 20.1 | WO2015197823 SEQ ID NO: 273 | 21762 |
| | | variable | | | |
| | | region | | | |
| TAU785 | Tau | Light chain | Native 22.1 | WO2015197823 SEQ ID NO: 277 | 21763 |
| | | variable | | | |
| | | region | | | |
| TAU786 | Tau | Light chain | Native 24.1 | WO2015197823 SEQ ID NO: 280 | 21764 |
| | | variable | | | |
| | | region | | | |
| TAU787 | Tau | Light chain | Native 27.1 | WO2015197823 SEQ ID NO: 283 | 21765 |
| | | variable | | | |
| | | region | | | |
| TAU788 | Tau | Light chain | Native 28.1 | WO2015197823 SEQ ID NO: 285 | 21766 |
| | | variable | | | |
| | | region | | | |
| TAU789 | Tau | Light chain | Native 41.1 | WO2015197823 SEQ ID NO: 288 | 21767 |
| | | variable | | | |
| | | region | | | |
| TAU790 | Tau | Light chain | Native 41.2; | WO2015197823 SEQ ID NO: 290; | 21768 |
| | | variable | Native 42.1 | WO2015197823 SEQ ID NO: 293 | |
| | | region | | | |
| TAU791 | Tau | Light chain | Native 43.1 | WO2015197823 SEQ ID NO: 296 | 21769 |
| | | variable | | | |
| | | region | | | |
| TAU792 | Tau | Light chain | Native 44.1 | WO2015197823 SEQ ID NO: 300 | 21770 |
| | | variable | | | |
| | | region | | | |
| TAU793 | Tau | Light chain | Native 45.1 | WO2015197823 SEQ ID NO: 304 | 21771 |
| | | variable | | | |
| | | region | | | |
| TAU794 | Tau | Light chain | Native 46.1 | WO2015197823 SEQ ID NO: 307 | 21772 |
| | | variable | | | |
| | | region | | | |
| TAU795 | Tau | Light chain | Native 47.1 | WO2015197823 SEQ ID NO: 310 | 21773 |
| | | variable | | | |
| | | region | | | |
| TAU796 | Tau | Light chain | Native 47.2 | WO2015197823 SEQ ID NO: 312 | 21774 |
| | | variable | | | |
| | | region | | | |
| TAU797 | Tau | Light chain | Native 49.1 | WO2015197823 SEQ ID NO: 314 | 21775 |
| | | variable | | | |
| | | region | | | |
| TAU798 | Tau | Light chain | 6B2G12 | WO2016007414 SEQ ID NO: 8 | 21776 |
| | | variable | | | |
| | | region | | | |
| TAU799 | Tau | Light chain | scFv235 | WO2016007414 SEQ ID NO: 10 | 21777 |
| | | variable | | | |
| | | region | | | |
| TAU800 | Tau | Light chain | | WO2015120364 SEQ ID NO: 24 | 21778 |
| | | variable | | | |
| | | region | | | |
| TAU801 | Tau | Light chain | | WO2015120364 SEQ ID NO: 36 | 21779 |
| | | variable | | | |
| | | region | | | |
| TAU802 | Tau | Light chain | pT231/pS235_1 | WO2014016737 SEQ ID NO: 14 | 21780 |
| | | variable | | | |
| | | region | | | |
| TAU803 | Tau | Light chain | pT231/pS235_2 | WO2014016737 SEQ ID NO: 16 | 21781 |
| | | variable | | | |
| | | region | | | |
| TAU804 | Tau | Light chain | pT212/pS214_1 | WO2014016737 SEQ ID NO: 18 | 21782 |
| | | variable | | | |
| | | region | | | |
| TAU805 | Tau | Light chain | pT212/pS214_2 | WO2014016737 SEQ ID NO: 20 | 21783 |
| | | variable | | | |
| | | region | | | |
| TAU806 | Tau | Light chain | pS396/pS404_1 | WO2014016737 SEQ ID NO: 22 | 21784 |
| | | variable | | | |
| | | region | | | |
| TAU807 | Tau | Light chain | pS396/pS404_2 | WO2014016737 SEQ ID NO: 24 | 21785 |
| | | variable | | | |
| | | region | | | |
| TAU808 | Tau | Light chain | 2H9 | WO2014096321 SEQ ID NO: 15 | 21786 |
| | | variable | | | |
| | | region | | | |
| TAU809 | Tau | Light chain | | WO2015122922 SEQ ID NO: 15 | 21787 |
| | | variable | | and 23 | |
| | | region | | | |
| TAU810 | Tau | Light chain | | WO2015122922 SEQ ID NO: 31 | 21788 |
| | | variable | | and 39 | |
| | | region | | | |
| TAU811 | Tau | Light chain | | WO2015122922 SEQ ID NO: 47 | 21789 |
| | | variable | | | |
| | | region | | | |
| TAU812 | Tau | Light chain | | WO2015122922 SEQ ID NO: 55 | 21790 |
| | | variable | | | |
| | | region | | | |
| TAU813 | Tau | Light chain | | WO2015122922 SEQ ID NO: 63 | 21791 |
| | | variable | | | |
| | | region | | | |
| TAU814 | Tau | Light chain | | WO2015122922 SEQ ID NO: 71 | 21792 |
| | | variable | | | |
| | | region | | | |
| TAU815 | Tau | light chain | 16B5 | US20160031976 SEQ ID NO: 16 | 21793 |
| | | variable | | | |
| | | region kappa | | | |
| TAU816 | Tau | light chain | | US20160031976 SEQ ID NO: 20 | 21794 |
| | | variable | | | |
| | | region kappa | | | |
| TAU817 | Tau | Light chain | NI-105.9C4 | US20150344553 SEQ ID NO: 77 | 21795 |
| | | variable | | | |
| | | region, before | | | |
| | | germlining | | | |
| TAU818 | Tau | Light chain | variant 1-VL21 | US20150175685 SEQ ID NO: 16; | 21796 |
| | | variable | | WO2015197735 SEQ ID NO: 16 | |
| | | region, | | | |
| | | humanized | | | |
| TAU819 | Tau | Light chain | variant 2-VL22 | US20150175685 SEQ ID NO: 17; | 21797 |
| | | variable | | WO2015197735 SEQ ID NO: 17 | |
| | | region, | | | |
| | | humanized | | | |
| TAU820 | Tau | Light chain | variant 4-VL01 | US20150175685 SEQ ID NO: 32 | 21798 |
| | | variable | | | |
| | | region, | | | |
| | | humanized | | | |
| TAU821 | Tau | Light chain | variant 5-VL09 | US20150175685 SEQ ID NO: 33 | 21799 |
| | | variable | | | |
| | | region, | | | |
| | | humanized | | | |
| TAU822 | Tau | Light chain | variant 6-VL12 | US20150175685 SEQ ID NO: 34 | 21800 |
| | | variable | | | |
| | | region, | | | |
| | | humanized | | | |
| TAU823 | Tau | Light chain | variant 7-VL15 | US20150175685 SEQ ID NO: 35 | 21801 |
| | | variable | | | |
| | | region, | | | |
| | | humanized | | | |
| TAU824 | Tau | Light chain | variant 8-VL16 | US20150175685 SEQ ID NO: 36 | 21802 |
| | | variable | | | |
| | | region, | | | |
| | | humanized | | | |
| TAU825 | Tau | Light chain | variant 9-VL17 | US20150175685 SEQ ID NO: 37 | 21803 |
| | | variable | | | |
| | | region, | | | |
| | | humanized | | | |
| TAU826 | Tau | Light chain | variant 10-VL19 | US20150175685 SEQ ID NO: 38 | 21804 |
| | | variable | | | |
| | | region, | | | |
| | | humanized | | | |
| TAU827 | Tau | Light chain | variant 11-VL28 | US20150175685 SEQ ID NO: 39 | 21805 |
| | | variable | | | |
| | | region, | | | |
| | | humanized | | | |
| TAU828 | Tau (pS422) | Light chain | variant 12-VL33 | US20150175685 SEQ ID NO: 40 | 21806 |
| | | variable | | | |
| | | region, | | | |
| | | humanized | | | |
| TAU829 | Tau | Light chain | variant 13-VL35 | US20150175685 SEQ ID NO: 41 | 21807 |
| | | variable | | | |
| | | region, | | | |
| | | humanized | | | |
| TAU830 | Tau | Light chain | variant 14-VL39 | US20150175685 SEQ ID NO: 42 | 21808 |
| | | variable | | | |
| | | region, | | | |
| | | humanized | | | |
| TAU831 | Tau | Light chain | variant 15-VL40 | US20150175685 SEQ ID NO: 43 | 21809 |
| | | variable | | | |
| | | region, | | | |
| | | humanized | | | |
| TAU832 | Tau | Light chain | variant 16-VL41 | US20150175685 SEQ ID NO: 44 | 21810 |
| | | variable | | | |
| | | region, | | | |
| | | humanized | | | |
| TAU833 | Tau | Light chain | variant 17-VL42 | US20150175685 SEQ ID NO: 45 | 21811 |
| | | variable | | | |
| | | region, | | | |
| | | humanized | | | |
| TAU834 | Tau | Light chain | variant 4-VH01 | US20150175685 SEQ ID NO: 46 | 21812 |
| | | variable | | | |
| | | region, | | | |
| | | humanized | | | |
| TAU835 | Tau | Light chain | variant 5-VH02 | US20150175685 SEQ ID NO: 47 | 21813 |
| | | variable | | | |
| | | region, | | | |
| | | humanized | | | |
| TAU836 | Tau | Light chain | variant 6-VH03 | US20150175685 SEQ ID NO: 48 | 21814 |
| | | variable | | | |
| | | region, | | | |
| | | humanized | | | |
| TAU837 | Tau | Light chain | variant 7-VH04 | US20150175685 SEQ ID NO: 49 | 21815 |
| | | variable | | | |
| | | region, | | | |
| | | humanized | | | |
| TAU838 | Tau | Light chain | variant 8-VH14 | US20150175685 SEQ ID NO: 50 | 21816 |
| | | variable | | | |
| | | region, | | | |
| | | humanized | | | |
| TAU839 | Tau | Light chain | variant 9-VH15 | US20150175685 SEQ ID NO: 51 | 21817 |
| | | variable | | | |
| | | region, | | | |
| | | humanized | | | |
| TAU840 | Tau | Light chain | variant 10-VH18 | US20150175685 SEQ ID NO: 52 | 21818 |
| | | variable | | | |
| | | region, | | | |
| | | humanized | | | |
| TAU841 | Tau | Light chain | variant 11-VH19 | US20150175685 SEQ ID NO: 53 | 21819 |
| | | variable | | | |
| | | region, | | | |
| | | humanized | | | |
| TAU842 | Tau | Light chain | variant 12-VH22 | US20150175685 SEQ ID NO: 54 | 21820 |
| | | variable | | | |
| | | region, | | | |
| | | humanized | | | |
| TAU843 | Tau | Light chain | variant 13-VH23 | US20150175685 SEQ ID NO: 55 | 21821 |
| | | variable | | | |
| | | region, | | | |
| | | humanized | | | |
| TAU844 | Tau | Light chain | variant 14-VH24 | US20150175685 SEQ ID NO: 56 | 21822 |
| | | variable | | | |
| | | region, | | | |
| | | humanized | | | |
| TAU845 | Tau | Light chain | variant 15-VH31 | US20150175685 SEQ ID NO: 57 | 21823 |
| | | variable | | | |
| | | region, | | | |
| | | humanized | | | |
| TAU846 | Tau | Light chain | IPN002 Vk | U.S. Pat. No. 8,980,270 SEQ ID NO: 40 | 21824 |
| | | variable | variant 1 | | |
| | | region, | | | |
| | | humanized | | | |
| TAU847 | Tau | Light chain | IPN002 Vk | U.S. Pat. No. 8,980,270 SEQ ID NO: 41 | 21825 |
| | | variable | variant 2 | | |
| | | region, | | | |
| | | humanized | | | |
| TAU848 | Tau | Light chain | IPN002 Vk | U.S. 8,980,270 SEQ ID NO: 42 | 21826 |
| | | variable | variant 3 | | |
| | | region, | | | |
| | | humanized | | | |
| TAU849 | Tau | Light chain | IPN002 VK | U.S. 8,980,270 SEQ ID NO: 43 | 21827 |
| | | variable | variant 4 | | |
| | | region, | | | |
| | | humanized | | | |
| TAU850 | Tau | Light chain | | US20160031976 SEQ ID NO: 21 | 21828 |
| | | variable | | | |
| | | region, | | | |
| | | mature | | | |
| TAU851 | Tau | Light chain | | US20160031976 SEQ ID NO: 22 | 21829 |
| | | variable | | | |
| | | region, | | | |
| | | mature | | | |
| TAU852 | Tau | Light chain | | US20160031976 SEQ ID NO: 23 | 21830 |
| | | variable | | | |
| | | region, | | | |
| | | mature | | | |
| TAU853 | Tau | ScFv | scFv235 | WO2016007414 SEQ ID NO: 18 | 21831 |
| TAU854 | Tau | scFv235 | | WO2016007414 SEQ ID NO: 22 | 21832 |
| | | Fusion | | | |
| | | Protein | | | |
| TAU855 | Tau | scFv235 | | WO2016007414 SEQ ID NO: 23 | 21833 |
| | | Fusion | | | |
| | | Protein | | | |
| TAU856 | Tau | scFv235 | | WO2016007414 SEQ ID NO: 24 | 21834 |
| | | Fusion | | | |
| | | Protein | | | |
| TAU857 | Tau | scFv235 | | WO2016007414 SEQ ID NO: 25 | 21835 |
| | | Fusion | | | |
| | | Protein | | | |
| TAU858 | Tau | scFv235 | | WO2016007414 SEQ ID NO: 26 | 21836 |
| | | Fusion | | | |
| | | Protein | | | |
| TAU859 | Tau | | Y15982 Igkv8- | WO2016079597 SEQ ID NO: 60 | 21837 |
| | | | 21*01 | | |
| TAU860 | Tau | | L17135 Igkv8- | WO2016079597 SEQ ID NO: 61 | 21838 |
| | | | 28*02 | | |
| TAU861 | Tau | | Y15980 IGKV8- | WO2016079597 SEQ ID NO: 62 | 21839 |
| | | | 19*01 | | |
| TAU862 | Tau | | AJ235948 | WO2016079597 SEQ ID NO: 63 | 21840 |
| | | | IGKV8-30*01 | | |
| TAU863 | Tau | | AJ235947 | WO2016079597 SEQ ID NO: 64 | 21841 |
| | | | IGKV8-28*01 | | |
| TAU864 | Tau | | X72449 | WO2016079597 SEQ ID NO: 65 | 21842 |
| TAU865 | Tau | | AC160990 | WO2016079597 SEQ ID NO: 66 | 21843 |
| | | | Musmus IGHV1- | | |
| | | | 81*01 | | |
| TAU866 | Tau | | AC160473 | WO2016079597 SEQ ID NO: 67 | 21844 |
| | | | Musmus IGHVI- | | |
| | | | 83*01 | | |
| TAU867 | Tau | | AC160990 | WO2016079597 SEQ ID NO: 68 | 21845 |
| | | | Musmus IGHV1- | | |
| | | | 83*01 | | |
| TAU868 | Tau | | AC160473 | WO2016079597 SEQ ID NO: 69 | 21846 |
| | | | Musmus IGHV1- | | |
| | | | 75*01 | | |
| TAU869 | Tau | | X02064 Musmus | WO2016079597 SEQ ID NO: 70 | 21847 |
| | | | IGHV1-54*02 | | |
| TAU870 | Tau | | M65092 | WO2016079597 SEQ ID NO: 71 | 21848 |
| TAU871 | Tau | | | US20150320860 SEQ ID NO: 56 | 21849 |
| TAU872 | Tau | | | US20150320860 SEQ ID NO: 57 | 21850 |
| TAU873 | Tau | | | US20150320860 SEQ ID NO: 58 | 21851 |
| TAU874 | Tau | | | US20150320860 SEQ ID NO: 59 | 21852 |
| TAU875 | Tau | Light chain | | US20150183855 SEQ ID NO: 14; | 21853 |
| | | variable | | WO2016126993 SEQ ID NO: 14 | |
| | | region | | | |
| TAU876 | Tau (O- | Heavy chain | | WO2014159244 SEQ ID NO: 1 | 21854 |
| | GlcNAc) | variable | | | |
| | | region | | | |
| TAU877 | Tau (O- | Light chain | | WO2014159244 SEQ ID NO: 2 | 21855 |
| | GlcNAc) | variable | | | |
| | | region | | | |
| TAU878 | Tau (pS422) | Heavy chain | | WO2015197735 SEQ ID NO: 58 | 21856 |
| | | constant | | | |
| | | region | | | |
| TAU879 | Tau (pS422) | Heavy chain | | WO2015197735 SEQ ID NO: 139 | 21857 |
| | | HC anti-TfR2 | | | |
| | | antibody | | | |
| | | conjugated to | | | |
| | | scFv anti- | | | |
| | | biotin | | | |
| | | antibody | | | |
| | | fragment | | | |
| TAU880 | Tau (pS422) | Heavy chain | | WO2015197735 SEQ ID NO: 138 | 21858 |
| | | HC anti-TfR2 | | | |
| | | antibody | | | |
| | | conjugated to | | | |
| | | scFv anti- | | | |
| | | digoxigenin | | | |
| | | antibody | | | |
| | | fragment | | | |
| TAU881 | Tau (pS422) | Heavy chain | | WO2015197735 SEQ ID NO: 135 | 21859 |
| | | HC anti-TfRI | | | |
| | | antibody | | | |
| | | conjugated to | | | |
| | | scFv anti- | | | |
| | | digoxigenin | | | |
| | | antibody | | | |
| | | fragment | | | |
| TAU882 | Tau (pS422) | Heavy chain | VH00 | WO2015197735 SEQ ID NO: 11; | 21860 |
| | | variable | | U.S. Pat. No. 9,290,567 SEQ ID NO: 54 | |
| | | region | | | |
| TAU883 | Tau (pS422) | Heavy chain | | WO2015197735 SEQ ID NO: 68 | 21861 |
| | | variable | | | |
| | | region | | | |
| TAU884 | Tau (pS422) | Heavy chain | | WO2015197735 SEQ ID NO: 76 | 21862 |
| | | variable | | | |
| | | region | | | |
| TAU885 | Tau (pS422) | Heavy chain | | WO2015197735 SEQ ID NO: 84 | 21863 |
| | | variable | | | |
| | | region | | | |
| TAU886 | Tau (pS422) | Heavy chain | | WO2015197735 SEQ ID NO: 92 | 21864 |
| | | variable | | | |
| | | region | | | |
| TAU887 | Tau (pS422) | Heavy chain | | WO2015197735 SEQ ID NO: 100 | 21865 |
| | | variable | | | |
| | | region | | | |
| TAU888 | Tau (pS422) | Heavy chain | | WO2015197735 SEQ ID NO: 108 | 21866 |
| | | variable | | | |
| | | region | | | |
| TAU889 | Tau (pS422) | Heavy chain | | WO2015197735 SEQ ID NO: 116 | 21867 |
| | | variable | | | |
| | | region | | | |
| TAU890 | Tau (pS422) | Heavy chain | | WO2015197735 SEQ ID NO: 129 | 21868 |
| | | variable | | | |
| | | region | | | |
| TAU891 | Tau (pS422) | Heavy chain | | WO2015197735 SEQ ID NO: 131 | 21869 |
| | | variable | | | |
| | | region | | | |
| TAU892 | Tau (pS422) | Heavy chain | | WO2015197735 SEQ ID NO: 148 | 21870 |
| | | variable | | | |
| | | region of the | | | |
| | | anti-HeliCar | | | |
| | | motif | | | |
| TAU893 | Tau (pS422) | Heavy | | WO2015197735 SEQ ID NO: 136 | 21871 |
| | | chainHC anti- | | | |
| | | TfRI antibody | | | |
| | | conjugated to | | | |
| | | scFv anti- | | | |
| | | biotin | | | |
| | | antibody | | | |
| | | fragment | | | |
| TAU894 | Tau (pS422) | Helicar motif | | WO2015197735 SEQ ID NO: 152 | 21872 |
| | | amino acid | | | |
| | | sequence | | | |
| | | cystein | | | |
| | | variant 1 | | | |
| | | fused to | | | |
| | | pseudomonas | | | |
| | | exotoxin | | | |
| | | LR8M with a | | | |
| | | GGG- | | | |
| | | peptidic | | | |
| | | linker and the | | | |
| | | C-terminal K | | | |
| | | deleted | | | |
| TAU895 | Tau (pS422) | human Ig- | | WO2015197735 SEQ ID NO: 60 | 21873 |
| | | lambda | | | |
| | | constant | | | |
| | | region | | | |
| TAU896 | Tau (pS422) | Light chain | | WO2015197735 SEQ ID NO: 137 | 21874 |
| | | LC anti-TIR2 | | | |
| | | antibody | | | |
| TAU897 | Tau (pS422) | Light chain | LC anti-TfR1 | WO2015197735 SEQ ID NO: 134 | 21875 |
| | | LC anti-TfRI | antibody | | |
| | | antibody | | | |
| TAU898 | Tau (pS422) | Light chain | VL00 | WO2015197735 SEQ ID NO: 7 | 21876 |
| | | variable | | | |
| | | region | | | |
| TAU899 | Tau (pS422) | Light chain | | WO2015197735 SEQ ID NO: 72 | 21877 |
| | | variable | | | |
| | | region | | | |
| TAU900 | Tau (pS422) | Light chain | | WO2015197735 SEQ ID NO: 80 | 21878 |
| | | variable | | | |
| | | region | | | |
| TAU901 | Tau (pS422) | Light chain | | WO2015197735 SEQ ID NO: 88 | 21879 |
| | | variable | | | |
| | | region | | | |
| TAU902 | Tau (pS422) | Light chain | | WO2015197735 SEQ ID NO: 96 | 21880 |
| | | variable | | | |
| | | region | | | |
| TAU903 | Tau (pS422) | Light chain | | WO2015197735 SEQ ID NO: 104 | 21881 |
| | | variable | | | |
| | | region | | | |
| TAU904 | Tau (pS422) | Light chain | | WO2015197735 SEQ ID NO: 112 | 21882 |
| | | variable | | | |
| | | region | | | |
| TAU905 | Tau (pS422) | Light chain | | WO2015197735 SEQ ID NO: 120 | 21883 |
| | | variable | | | |
| | | region | | | |
| TAU906 | Tau (pS422) | Light chain | | WO2015197735 SEQ ID NO: 130 | 21884 |
| | | variable | | | |
| | | region | | | |
| TAU907 | Tau (pS422) | Light chain | | WO2015197735 SEQ ID NO: 132 | 21885 |
| | | variable | | | |
| | | region | | | |
| TAU908 | Tau (pS422) | Light chain | | WO2015197735 SEQ ID NO: 151 | 21886 |
| | | variable | | | |
| | | region N51C | | | |
| | | variant of the | | | |
| | | anti-HeliCar | | | |
| | | motif | | | |
| TAU909 | Tau (pS422) | Light chain | | WO2015197735 SEQ ID NO: 150 | 21887 |
| | | variable | | | |
| | | region N55C | | | |
| | | variant of the | | | |
| | | anti-HeliCar | | | |
| | | motif | | | |
| TAU910 | Tau (pS422) | Light chain | | WO2015197735 SEQ ID NO: 149 | 21888 |
| | | variable | | | |
| | | region of the | | | |
| | | anti-HeliCar | | | |
| | | motif | | | |
| TAU911 | Tau pS422 | Heavy chain | | U.S. Pat. No. 9,290,567 SEQ ID NO: 13 | 21889 |
| | | constant | | | |
| | | region | | | |
| TAU912 | Tau pS422 | Heavy chain | | U.S. Pat. No. 9,290,567 SEQ ID NO: 14 | 21890 |
| | | constant | | | |
| | | region | | | |
| TAU913 | Tau pS422 | Heavy chain | | U.S. Pat. No. 9,290,567 SEQ ID NO: 15 | 21891 |
| | | constant | | | |
| | | region | | | |
| TAU914 | Tau pS422 | Heavy chain | | U.S. Pat. No. 9,290,567 SEQ ID NO: 16 | 21892 |
| | | constant | | | |
| | | region | | | |
| TAU915 | Tau pS422 | Heavy chain | Mab2.10.3 | U.S. Pat. No. 9,290,567 SEQ ID NO: 2 | 21893 |
| | | variable | | | |
| | | region | | | |
| TAU916 | Tau pS422 | Heavy chain | Mab 005 | U.S. Pat. No. 9,290,567 SEQ ID NO: 22 | 21894 |
| | | variable | | | |
| | | region | | | |
| TAU917 | Tau pS422 | Heavy chain | Mab 019 | U.S. Pat. No. 9,290,567 SEQ ID NO: 30 | 21895 |
| | | variable | | | |
| | | region | | | |
| TAU918 | Tau pS422 | Heavy chain | Mab 020 | U.S. Pat. No. 9,290,567 SEQ ID NO: 38 | 21896 |
| | | variable | | | |
| | | region | | | |
| TAU919 | Tau pS422 | Heavy chain | Mab 085 | U.S. Pat. No. 9,290,567 SEQ ID NO: 46 | 21897 |
| | | variable | | | |
| | | region | | | |
| TAU920 | Tau pS422 | Heavy chain | Mab 097 | U.S. Pat. No. 9,290,567 SEQ ID NO: 62 | 21898 |
| | | variable | | | |
| | | region | | | |
| TAU921 | Tau pS422 | Light chain | Mab2.10.3 | U.S. Pat. No. 9,290,567 SEQ ID NO: 1 | 21899 |
| | | variable | | | |
| | | region | | | |
| TAU922 | Tau pS422 | Light chain | Mab 005 | U.S. Pat. No. 9,290,567 SEQ ID NO: 26 | 21900 |
| | | variable | | | |
| | | region | | | |
| TAU923 | Tau pS422 | Light chain | Mab 019 | U.S. Pat. No. 9,290,567 SEQ ID NO: 34 | 21901 |
| | | variable | | | |
| | | region | | | |
| TAU924 | Tau pS422 | Light chain | Mab 020 | U.S. Pat. No. 9,290,567 SEQ ID NO: 42 | 21902 |
| | | variable | | | |
| | | region | | | |
| TAU925 | Tau pS422 | Light chain | Mab 085 | U.S. Pat. No. 9,290,567 SEQ ID NO: 50 | 21903 |
| | | variable | | | |
| | | region | | | |
| TAU926 | Tau pS422 | Light chain | Mab 086 | U.S. Pat. No. 9,290,567 SEQ ID NO: 58 | 21904 |
| | | variable | | | |
| | | region | | | |
| TAU927 | Tau pS422 | Light chain | Mab 097 | U.S. Pat. No. 9,290,567 SEQ ID NO: 66 | 21905 |
| | | variable | | | |
| | | region | | | |
| TAU928 | Tau/Amyloid | Heavy chain | 3.F5 | US20100323905 SEQ ID NO: 13 | 21906 |
| | beta/Alpha | variable | | and 119 | |
| | synuclein | region | | | |
| | | antibody | | | |
| TAU929 | Tau/Amyloid | Heavy chain | 3.A9 | US20100323905 SEQ ID NO: 14 | 21907 |
| | beta/Alpha | variable | | and 120 | |
| | synuclein | region | | | |
| | | antibody | | | |
| TAU930 | Tau/Amyloid | Heavy chain | 3.00E+09 | US20100323905 SEQ ID NO: 15, | 21908 |
| | beta/Alpha | variable | | 110 | |
| | synuclein | region | | | |
| | | antibody | | | |
| TAU931 | Tau/Amyloid | Heavy chain | #08 | US20100323905 SEQ ID NO: 16 | 21909 |
| | beta/Alpha | variable | | and 111 | |
| | synuclein | region | | | |
| | | antibody | | | |
| TAU932 | Tau/Amyloid | Heavy chain | VHH29 | US20100323905 SEQ ID NO: 18, | 21910 |
| | beta/Alpha | variable | | 118 | |
| | synuclein | region | | | |
| | | antibody | | | |
| TAU933 | Tau/Amyloid | Heavy chain | VHH07 | US20100323905 SEQ ID NO: 97, | 21911 |
| | beta/Alpha | variable | | 98 | |
| | synuclein | region | | | |
| | | antibody | | | |
| TAU934 | Tau/Amyloid | Heavy chain | VHH15 | US20100323905 SEQ ID NO: 99- | 21912 |
| | beta/Alpha | variable | | 101 | |
| | synuclein | region | | | |
| | | antibody | | | |
| TAU935 | Tau/Amyloid | Heavy chain | VHH01 | US20100323905 SEQ ID NO: 102 | 21913 |
| | beta/Alpha | variable | | | |
| | synuclein | region | | | |
| | | antibody | | | |
| TAU936 | Tau/Amyloid | Heavy chain | VHH04 | US20100323905 SEQ ID NO: 103 | 21914 |
| | beta/Alpha | variable | | | |
| | synuclein | region | | | |
| | | antibody | | | |
| TAU937 | Tau/Amyloid | Heavy chain | VHH19 | US20100323905 SEQ ID NO: 104 | 21915 |
| | beta/Alpha | variable | | | |
| | synuclein | region | | | |
| | | antibody | | | |
| TAU938 | Tau/Amyloid | Heavy chain | VHH21 | US20100323905 SEQ ID NO: 105 | 21916 |
| | beta/Alpha | variable | | | |
| | synuclein | region | | | |
| | | antibody | | | |
| TAU939 | Tau/Amyloid | Heavy chain | VHH05 | US20100323905 SEQ ID NO: 106 | 21917 |
| | beta/Alpha | variable | | | |
| | synuclein | region | | | |
| | | antibody | | | |
| TAU940 | Tau/Amyloid | Heavy chain | VHH23 | US20100323905 SEQ ID NO: 107 | 21918 |
| | beta/Alpha | variable | | | |
| | synuclein | region | | | |
| | | antibody | | | |
| TAU941 | Tau/Amyloid | Heavy chain | VHH34 | US20100323905 SEQ ID NO: 108 | 21919 |
| | beta/Alpha | variable | | | |
| | sy nuclein | region | | | |
| | | antibody | | | |
| TAU942 | Tau/Amyloid | Heavy chain | VHH26 | US20100323905 SEQ ID NO: 109 | 21920 |
| | beta/Alpha | variable | | | |
| | sy nuclein | region | | | |
| | | antibody | | | |
| TAU943 | Tau/Amyloid | Heavy chain | VHH18 | US20100323905 SEQ ID NO: 17 | 21921 |
| | beta/Alpha | variable | | and 112 | |
| | synuclein | region | | | |
| | | antibody | | | |
| TAU944 | Tau/Amyloid | Heavy chain | VHH09 | US20100323905 SEQ ID NO: 113 | 21922 |
| | beta/Alpha | variable | | | |
| | synuclein | region | | | |
| | | antibody | | | |
| TAU945 | Tau/Amyloid | Heavy chain | VHH20 | US20100323905 SEQ ID NO: 114 | 21923 |
| | beta/Alpha | variable | | | |
| | synuclein | region | | | |
| | | antibody | | | |
| TAU946 | Tau/Amyloid | Heavy chain | VHH32 | US20100323905 SEQ ID NO: 115 | 21924 |
| | beta/Alpha | variable | | | |
| | synuclein | region | | | |
| | | antibody | | | |
| TAU947 | Tau/Amyloid | Heavy chain | VHH30 | US20100323905 SEQ ID NO: 116 | 21925 |
| | beta/Alpha | variable | | | |
| | synuclein | region | | | |
| | | antibody | | | |
| TAU948 | Tau/Amyloid | Heavy chain | VHH28 | US20100323905 SEQ ID NO: 117 | 21926 |
| | beta/Alpha | variable | | | |
| | synuclein | region | | | |
| | | antibody | | | |
| TAU949 | Tau/Amyloid | Heavy chain | VHH14 | US20100323905 SEQ ID NO: 121 | 21927 |
| | beta/Alpha | variable | | | |
| | synuclein | region | | | |
| | | antibody | | | |
| TAU950 | Tau/Amyloid | Heavy chain | VHH12 | US20100323905 SEQ ID NO: 122 | 21928 |
| | beta/Alpha | variable | | | |
| | synuclein | region | | | |
| | | antibody | | | |
| TAU951 | Tau/Amyloid | Heavy chain | 1B | US20100323905 SEQ ID NO: 52 | 21929 |
| | beta/Alpha | variable | | | |
| | synuclein | region | | | |
| | | antibody, | | | |
| | | amyloid 42 | | | |
| | | VHH | | | |
| TAU952 | Tau/Amyloid | Heavy chain | 1D | US20100323905 SEQ ID NO: 53 | 21930 |
| | beta/Alpha | variable | | | |
| | synuclein | region | | | |
| | | antibody, | | | |
| | | amyloid 42 | | | |
| | | VHH | | | |
| TAU953 | Tau/Amyloid | Heavy chain | 2A | US20100323905 SEQ ID NO: 54 | 21931 |
| | beta/Alpha | variable | | | |
| | synuclein | region | | | |
| | | antibody, | | | |
| | | amyloid 42 | | | |
| | | VHH | | | |
| TAU954 | Tau/Amyloid | Heavy chain | 2B | US20100323905 SEQ ID NO: 55 | 21932 |
| | beta/Alpha | variable | | | |
| | synuclein | region | | | |
| | | antibody, | | | |
| | | amyloid 42 | | | |
| | | VHH | | | |
| TAU955 | Tau/Amyloid | Heavy chain | 2F | US20100323905 SEQ ID NO: 56 | 21933 |
| | beta/Alpha | variable | | | |
| | synuclein | region | | | |
| | | antibody, | | | |
| | | amyloid 42 | | | |
| | | VHH | | | |
| TAU956 | Tau/Amyloid | Heavy chain | 3A | US20100323905 SEQ ID NO: 57 | 21934 |
| | beta/Alpha | variable | | | |
| | synuclein | region | | | |
| | | antibody, | | | |
| | | amyloid 42 | | | |
| | | VHH | | | |
| TAU957 | Tau/Amyloid | Heavy chain | 3H | US20100323905 SEQ ID NO: 58 | 21935 |
| | beta/Alpha | variable | | | |
| | sy nuclein | region | | | |
| | | antibody, | | | |
| | | amyloid 42 | | | |
| | | VHH | | | |
| TAU958 | Tau/Amyloid | Heavy chain | 4C | US20100323905 SEQ ID NO: 59 | 21936 |
| | beta/Alpha | variable | | | |
| | synuclein | region | | | |
| | | antibody, | | | |
| | | amyloid 42 | | | |
| | | VHH | | | |
| TAU959 | Tau/Amyloid | Heavy chain | 8F | US20100323905 SEQ ID NO: 60 | 21937 |
| | beta/Alpha | variable | | | |
| | synuclein | region | | | |
| | | antibody, | | | |
| | | amyloid 42 | | | |
| | | VHH | | | |
| TAU960 | Tau/Amyloid | Heavy chain | 11D | US20100323905 SEQ ID NO: 61 | 21938 |
| | beta/Alpha | variable | | | |
| | synuclein | region | | | |
| | | antibody, | | | |
| | | amyloid 42 | | | |
| | | VHH | | | |
| TAU961 | Tau/Amyloid | Heavy chain | EME7E | US20100323905 SEQ ID NO: 62 | 21939 |
| | beta/Alpha | variable | | | |
| | synuclein | region | | | |
| | | antibody, | | | |
| | | VHH for | | | |
| | | emerin | | | |
| TAU962 | Tau/Amyloid | Heavy chain | EME1C | US20100323905 SEQ ID NO: 63 | 21940 |
| | beta/Alpha | variable | | | |
| | synuclein | region | | | |
| | | antibody, | | | |
| | | VHH for | | | |
| | | emerin | | | |
| TAU963 | Tau/Amyloid | Heavy chain | VHH01 | US20100323905 SEQ ID NO: 64 | 21941 |
| | beta/Alpha | variable | | | |
| | synuclein | region | | | |
| | | antibody, | | | |
| | | VHH for | | | |
| | | emerin | | | |
| TAU964 | Tau/Amyloid | Heavy chain | VHH03 / | US20100323905 SEQ ID NO: 65 | 21942 |
| | beta/Alpha | variable | VHH23 | | |
| | synuclein | region | | | |
| | | antibody, | | | |
| | | VHH for | | | |
| | | emerin | | | |
| TAU965 | Tau/Amyloid | Heavy chain | EME3H | US20100323905 SEQ ID NO: 66 | 21943 |
| | beta/Alpha | variable | | | |
| | synuclein | region | | | |
| | | antibody, | | | |
| | | VHH for | | | |
| | | emerin | | | |
| TAU966 | Tau/Amyloid | Heavy chain | VHH09 | US20100323905 SEQ ID NO: 67 | 21944 |
| | beta/Alpha | variable | | | |
| | synuclein | region | | | |
| | | antibody, | | | |
| | | VHH for | | | |
| | | emerin | | | |
| TAU967 | Tau/Amyloid | Heavy chain | VHH12 | US20100323905 SEQ ID NO: 68 | 21945 |
| | beta/Alpha | variable | | | |
| | synuclein | region | | | |
| | | antibody | | | |
| | | VHH for | | | |
| | | emerin | | | |
| TAU968 | Tau/Amyloid | Heavy chain | VHH05 | US20100323905 SEQ ID NO: 69 | 21946 |
| | beta/Alpha | variable | | | |
| | synuclein | region | | | |
| | | antibody, | | | |
| | | VHH for | | | |
| | | emerin | | | |
| TAU969 | Tau/Amyloid | Heavy chain | VHH11 | US20100323905 SEQ ID NO: 70 | 21947 |
| | beta/Alpha | variable | | | |
| | synuclein | region | | | |
| | | antibody, | | | |
| | | VHH for | | | |
| | | emerin | | | |
| TAU970 | Tau/Amyloid | Heavy chain | EME8A | US20100323905 SEQ ID NO: 71 | 21948 |
| | beta/Alpha | variable | | | |
| | synuclein | region | | | |
| | | antibody, | | | |
| | | VHH for | | | |
| | | emerin | | | |
| TAU971 | Tau/Amyloid | Heavy chain | VHH02 | US20100323905 SEQ ID NO: 72 | 21949 |
| | beta/Alpha | variable | | | |
| | synuclein | region | | | |
| | | antibody, | | | |
| | | VHH for | | | |
| | | emerin | | | |
| TAU972 | Tau/Amyloid | Heavy chain | VHH15 | US20100323905 SEQ ID NO: 73 | 21950 |
| | beta/Alpha | variable | | | |
| | synuclein | region | | | |
| | | antibody, | | | |
| | | VHH for | | | |
| | | emerin | | | |
| TAU973 | Tau/Amyloid | Heavy chain | VHH10 | US20100323905 SEQ ID NO: 74 | 21951 |
| | beta/Alpha | variable | | | |
| | synuclein | region | | | |
| | | antibody, | | | |
| | | VHH for | | | |
| | | emerin | | | |
| TAU974 | Tau/Amyloid | Heavy chain | EME4B | US20100323905 SEQ ID NO: 75 | 21952 |
| | beta/Alpha | variable | | | |
| | synuclein | region | | | |
| | | antibody, | | | |
| | | VHH for | | | |
| | | emerin | | | |
| TAU975 | Tau/Amyloid | Heavy chain | VHH13 | US20100323905 SEQ ID NO: 76 | 21953 |
| | beta/Alpha | variable | | | |
| | synuclein | region | | | |
| | | antibody, | | | |
| | | VHH for | | | |
| | | emerin | | | |
| TAU976 | Tau/Amyloid | Heavy chain | EME7F | US20100323905 SEQ ID NO: 77 | 21954 |
| | beta/Alpha | variable | | | |
| | synuclein | region | | | |
| | | antibody, | | | |
| | | VHH for | | | |
| | | emerin | | | |
| TAU977 | Tau/Amyloid | Heavy chain | VHH14 | US20100323905 SEQ ID NO: 78 | 21955 |
| | beta/Alpha | variable | | | |
| | synuclein | region | | | |
| | | antibody, | | | |
| | | VHH for | | | |
| | | emerin | | | |
| TAU978 | Tau/Amyloid | Heavy chain | EME2G | US20100323905 SEQ ID NO: 79 | 21956 |
| | beta/Alpha | variable | | | |
| | synuclein | region | | | |
| | | antibody, | | | |
| | | VHH for | | | |
| | | emerin | | | |
| TAU979 | Tau/Amyloid | Heavy chain | EME8D | US20100323905 SEQ ID NO: 80 | 21957 |
| | beta/Alpha | variable | | | |
| | synuclein | region | | | |
| | | antibody, | | | |
| | | VHH for | | | |
| | | emerin | | | |
| TAU980 | Tau/Amyloid | Heavy chain | VHH04 | US20100323905 SEQ ID NO: 81 | 21958 |
| | beta/Alpha | variable | | | |
| | synuclein | region | | | |
| | | antibody, | | | |
| | | VHH for | | | |
| | | emerin | | | |
| TAU981 | Tau/Amyloid | Heavy chain | VHH07 / | US20100323905 SEQ ID NO: 82 | 21959 |
| | beta/Alpha | variable | VHH08 | | |
| | synuclein | region | | | |
| | | antibody, | | | |
| | | VHH for | | | |
| | | emerin | | | |
| TAU982 | Tau/Amyloid | Heavy chain | VHH16 | US20100323905 SEQ ID NO: 83 | 21960 |
| | beta/Alpha | variable | | | |
| | synuclein | region | | | |
| | | antibody, | | | |
| | | VHH for | | | |
| | | emerin | | | |
| TAU983 | Tau/Amyloid | Heavy chain | 3.6B | US20100323905 SEQ ID NO: 84 | 21961 |
| | beta/Alpha | variable | | | |
| | synuclein | region | | | |
| | | antibody, | | | |
| | | VHH for | | | |
| | | emerin | | | |
| TAU984 | Tau/Amyloid | Heavy chain | 3.8B | US20100323905 SEQ ID NO: 85 | 21962 |
| | beta/Alpha | variable | | | |
| | synuclein | region | | | |
| | | antibody, | | | |
| | | VHH for | | | |
| | | emerin | | | |
| TAU985 | Tau/Amyloid | Heavy chain | VHH24 | US20100323905 SEQ ID NO: 86 | 21963 |
| | beta/Alpha | variable | | | |
| | synuclein | region | | | |
| | | antibody, | | | |
| | | VHH for | | | |
| | | emerin | | | |
| TAU986 | Tau/Amyloid | Heavy chain | VHH21 | US20100323905 SEQ ID NO: 87 | 21964 |
| | beta/Alpha | variable | | | |
| | synuclein | region | | | |
| | | antibody, | | | |
| | | VHH for | | | |
| | | emerin | | | |
| TAU987 | Tau/Amyloid | Heavy chain | 3.8E | US20100323905 SEQ ID NO: 88 | 21965 |
| | beta/Alpha | variable | | | |
| | synuclein | region | | | |
| | | antibody. | | | |
| | | VHH for | | | |
| | | emerin | | | |
| TAU988 | Tau/Amyloid | Heavy chain | | US20100323905 SEQ ID NO: 89 | 21966 |
| | beta/Alpha | variable | | | |
| | synuclein | region | | | |
| | | antibody, | | | |
| | | VHH which | | | |
| | | can | | | |
| | | translocate | | | |
| | | via blood | | | |
| | | brain barrier | | | |
| TAU989 | Tau/Amyloid | Heavy chain | | US20100323905 SEQ ID NO: 90 | 21967 |
| | beta/Alpha | variable | | | |
| | synuclein | region | | | |
| | | antibody, | | | |
| | | VHH which | | | |
| | | can | | | |
| | | translocate | | | |
| | | via blood | | | |
| | | brain barrier | | | |
| TAU990 | Tau/Aß | | | US20110002945 SEQ ID NO: 2 | 21968 |
| | peptides | | | | |
| TAU991 | Tau/Aß | | | US20110002945 SEQ ID NO: 3 | 21969 |
| | peptides | | | | |
| TAU992 | Tau | CDR | | WO2016137811 SEQ ID NO: 3 | 21970 |
| TAU993 | Tau | CDR | | WO2016137811 SEQ ID NO: 4 | 21971 |
| TAU994 | Tau | CDR | | WO2016137811 SEQ ID NO: 5 | 21972 |
| TAU995 | Tau | CDR | | WO2016137811 SEQ ID NO: 6 | 21973 |
| TAU996 | Tau | CDR | | WO2016137811 SEQ ID NO: 7 | 21974 |
| TAU997 | Tau | CDR | | WO2016137811 SEQ ID NO: 8 | 21975 |
| TAU998 | Tau | CDR | | WO2015122922 SEQ ID NO: 41 | 21976 |
| TAU999 | Tau | CDR | | WO2015122922 SEQ ID NO: 49 | 21977 |
| | | | | and 57 | |
| TAU1000 | Tau | CDR | | WO2016126993 SEQ ID NO: 16 | 21978 |
| TAU1001 | Tau | CDR | | WO2016126993 SEQ ID NO: 17 | 21979 |
| TAU1002 | Tau | CDR | | WO2016126993 SEQ ID NO: 18 | 21980 |
| TAU1003 | Tau | CDR | | WO2016126993 SEQ ID NO: 19 | 21981 |
| TAU1004 | Tau | CDR | | WO2016126993 SEQ ID NO: 20 | 21982 |
| TAU1005 | Tau | CDR | | WO2016126993 SEQ ID NO: 21 | 21983 |
| TAU1006 | Tau | dimeric | DH-Tau15 | WO2016055941 SEQ ID NO: 20 | 21984 |
| | | antibody | | | |
| TAU1007 | Tau | Fc | Fc-Tau15 | WO2016055941 SEQ ID NO: 23 | 21985 |
| TAU1008 | Tau | full antibody | MC-1 Furin 2A | WO2015035190 SEQ ID NO: 2 | 21986 |
| TAU1009 | Tau | full antibody | MC-1 Furin 2A | WO2015035190 SEQ ID NO: 4 | 21987 |
| TAU1010 | Tau | full antibody | MC-1 optimized | WO2015035190 SEQ ID NO: 6 | 21988 |
| | | | seq | | |
| TAU1011 | Tau | full antibody | PHF-1 Furin 2A | WO2015035190 SEQ ID NO: 1 | 21989 |
| TAU1012 | Tau | full antibody | PHF-1 Furin 2A | WO2015035190 SEQ ID NO: 3 | 21990 |
| TAU1013 | Tau | full antibody | PHF-1 optimized | WO2015035190 SEQ ID NO: 5 | 21991 |
| | | | seq | | |
| TAU1014 | Tau | Heavy chain | 1 A6 | WO2016137950 SEQ ID NO: 46 | 21992 |
| TAU1015 | Tau | heavy chain | 113F5-F7 | WO2016196726 SEQ ID NO: 90 | 21993 |
| TAU1016 | Tau | heavy chain | 11E10-B8 | WO2016196726 SEQ ID NO: 30 | 21994 |
| TAU1017 | Tau | heavy chain | 123E9-A1 | WO2016196726 SEQ ID NO: 140 | 21995 |
| TAU1018 | Tau | heavy chain | 125B11-H3 | WO2016196726 SEQ ID NO: 80 | 21996 |
| TAU1019 | Tau | heavy chain | 126F11-G11 | WO2016196726 SEQ ID NO: 180 | 21997 |
| TAU1020 | Tau | heavy chain | 12A10-E8 | WO2016196726 SEQ ID NO: 250 | 21998 |
| TAU1021 | Tau | heavy chain | 14F5-D9 | WO2016196726 SEQ ID NO: 210 | 21999 |
| TAU1022 | Tau | heavy chain | 15C6-A7 | WO2016196726 SEQ ID NO: 150 | 22000 |
| TAU1023 | Tau | Heavy chain | 17H3.2 | WO2016112078 SEQ ID NO: 20 | 22001 |
| TAU1024 | Tau | heavy chain | 19F8-B1 | WO2016196726 SEQ ID NO: 160 | 22002 |
| TAU1025 | Tau | heavy chain | 19H6-F7 | WO2016196726 SEQ ID NO: 60 | 22003 |
| TAU1026 | Tau | heavy chain | 22G7-C9 | WO2016196726 SEQ ID NO: 230 | 22004 |
| TAU1027 | Tau | heavy chain | 24A11-D5 | WO2016196726 SEQ ID NO: 170 | 22005 |
| TAU1028 | Tau | heavy chain | 26C1-B11 and | WO2016196726 SEQ ID NO: 100 | 22006 |
| | | | 26C1-C8 | and 110 | |
| TAU1029 | Tau | Heavy chain | 29H2.10 | WO2016112078 SEQ ID NO: 22 | 22007 |
| TAU1030 | Tau | Heavy chain | 29H2.10N31S | WO2016112078 SEQ ID NO: 23 | 22008 |
| | | | (Mutant) | | |
| TAU1031 | Tau | heavy chain | 30G1-B2 | WO2016196726 SEQ ID NO: 120 | 22009 |
| TAU1032 | Tau | heavy chain | 37D3-H9 and | WO2016196726 SEQ ID NO: 10 | 22010 |
| | | | 37D3-H9b | and 20 | |
| TAU1033 | Tau | heavy chain | 3A4-H4 | WO2016196726 SEQ ID NO: 50 | 22011 |
| TAU1034 | Tau | Heavy chain | 4G11 | WO2016137950 SEQ ID NO: 42 | 22012 |
| TAU1035 | Tau | heavy chain | 52F6-F11 | WO2016196726 SEQ ID NO: 270 | 22013 |
| TAU1036 | Tau | heavy chain | 54C1-H11 and | WO2016196726 SEQ ID NO: 40 | 22014 |
| | | | 61E7-C4 | | |
| TAU1037 | Tau | heavy chain | 55E7-F11 | WO2016196726 SEQ ID NO: 260 | 22015 |
| TAU1038 | Tau | heavy chain | 66F5-A1 | WO2016196726 SEQ ID NO: 130 | 22016 |
| TAU1039 | Tau | heavy chain | 73H6-B8 | WO2016196726 SEQ ID NO: 220 | 22017 |
| TAU1040 | Tau | heavy chain | 7A11-C12 | WO2016196726 SEQ ID NO: 240 | 22018 |
| TAU1041 | Tau | heavy chain | 89F4-A1 | WO2016196726 SEQ ID NO: 190 | 22019 |
| TAU1042 | Tau | heavy chain | 93A8-D2 | WO2016196726 SEQ ID NO: 200 | 22020 |
| TAU1043 | Tau | heavy chain | 94B2-C1 | WO2016196726 SEQ ID NO: 70 | 22021 |
| TAU1044 | Tau ps409 | heavy chain | hAC1-36-3A8- | US20150175682 SEQ ID NO: 11 | 22022 |
| | | | Ab1 | | |
| TAU1045 | Tau | heavy chain | hu125B11.v17 | WO2016196726 SEQ ID NO: 310 | 22023 |
| | | | and hu125B11- | and 448 | |
| | | | H3.HC3 | | |
| TAU1046 | Tau | heavy chain | hu 125B11.v17 | WO2016196726 SEQ ID NO: 311 | 22024 |
| TAU1047 | Tau | heavy chain | hu125B11.v26 | WO2016196726 SEQ ID NO: 320 | 22025 |
| TAU1048 | Tau | heavy chain | hu125B11.v26 | WO2016196726 SEQ ID NO: 321 | 22026 |
| TAU1049 | Tau | heavy chain | hu125B11.v28 | WO2016196726 SEQ ID NO: 330 | 22027 |
| TAU1050 | Tau | heavy chain | hu125B11.v28 | WO2016196726 SEQ ID NO: 331 | 22028 |
| TAU1051 | Tau | heavy chain | hu125B11- | WO2016196726 SEQ ID NO: 446 | 22029 |
| | | | H3.HC1 | | |
| TAU1052 | Tau | heavy chain | hu 125B11- | WO2016196726 SEQ ID NO: 447 | 22030 |
| | | | H3.HC2 | | |
| TAU1053 | Tau | heavy chain | hu 125B11- | WO2016196726 SEQ ID NO: 449 | 22031 |
| | | | H3.HC4 | | |
| TAU1054 | Tau | heavy chain | hu125B11- | WO2016196726 SEQ ID NO: 450 | 22032 |
| | | | H3.HC5 | | |
| TAU1055 | Tau | heavy chain | hu125B11- | WO2016196726 SEQ ID NO: 451 | 22033 |
| | | | H3.HC6 | | |
| TAU1056 | Tau | heavy chain | Hu37D3.v39 | WO2016196726 SEQ ID NO: 560, | 22034 |
| | | | | 570, 580 | |
| TAU1057 | Tau | heavy chain | Hu37D3-H9.v1 | WO2016196726 SEQ ID NO: 280 | 22035 |
| TAU1058 | Tau | heavy chain | Hu37D3- | WO2016196726 SEQ ID NO: 340 | 22036 |
| | | | H9.v28.A4 | | |
| TAU1059 | Tau | heavy chain | Hu37D3- | WO2016196726 SEQ ID NO: 348 | 22037 |
| | | | H9.v28.A4 | | |
| | | | IgG4- | | |
| | | | S228P.YTE | | |
| TAU1060 | Tau | heavy chain | Hu37D3- | WO2016196726 SEQ ID NO: 602 | 22038 |
| | | | H9.v28.A4 | | |
| | | | lgG4- | | |
| | | | S228P.YTE des- | | |
| | | | K | | |
| TAU1061 | Tau | heavy chain | Hu37D3-H9.v5 | WO2016196726 SEQ ID NO: 290 | 22039 |
| TAU1062 | Tau | heavy chain | Hu94B2.HC1 | WO2016196726 SEQ ID NO: 452 | 22040 |
| TAU1063 | Tau | heavy chain | Hu94B2.HC2 | WO2016196726 SEQ ID NO: 453 | 22041 |
| TAU1064 | Tau | heavy chain | Hu94B2.HC3 | WO2016196726 SEQ ID NO: 454 | 22042 |
| TAU1065 | Tau | heavy chain | Hu94B2.HC4 | WO2016196726 SEQ ID NO: 455 | 22043 |
| TAU1066 | Tau | heavy chain | Hu94B2.HC5 | WO2016196726 SEQ ID NO: 456 | 22044 |
| TAU1067 | Tau | heavy chain | Hu94B2.HC6 | WO2016196726 SEQ ID NO: 457 | 22045 |
| TAU1068 | Tau | heavy chain | Hu94B2.HC7 | WO2016196726 SEQ ID NO: 458 | 22046 |
| TAU1069 | Tau | heavy chain | Hu94B2.HC8 | WO2016196726 SEQ ID NO: 459 | 22047 |
| TAU1070 | Tau | heavy chain | Hu94B2.v105 | WO2016196726 SEQ ID NO: 300 | 22048 |
| TAU1071 | Tau(pS422) | heavy chain | MAb086 | WO2015197735 SEQ ID NO: 11 | 22049 |
| TAU1072 | Tau | heavy chain | | WO2016137811 SEQ ID NO: 2 | 22050 |
| TAU1073 | Tau | heavy chain | | WO2016137811 SEQ ID NO: 12 | 22051 |
| TAU1074 | Tau (pS422) | heavy chain | | WO2015197735 SEQ ID NO: 58 | 22052 |
| | | constant | | | |
| | | regions | | | |
| TAU1075 | Tau | heavy chain | DC8E8 | WO2016079597 SEQ ID NO: 7 | 22053 |
| | | variable | | | |
| | | domain | | | |
| TAU1076 | Tau(pS422) | heavy chain | | WO2015197735 SEQ ID NO: 21 | 22054 |
| | | variable | | | |
| | | domain | | | |
| TAU1077 | Tau | heavy chain | | WO2016137811 SEQ ID NO: 10 | 22055 |
| | | variable | | | |
| | | domain | | | |
| TAU1078 | Tau & | intrabody | A2 | WO2014193632 SEQ ID NO: 2 | 22056 |
| | huntingtin | | | | |
| TAU1079 | Tau & | intrabody | E10 | WO2014193632 SEQ ID NO: 3 | 22057 |
| | huntingtin | | | | |
| TAU1080 | Tau & | intrabody | H8 | WO2014193632 SEQ ID NO: 4 | 22058 |
| | huntingtin | | | | |
| TAU1081 | Tau & | intrabody | TNT41 | WO2014193632 SEQ ID NO: 1 | 22059 |
| | huntingtin | | | | |
| TAU1082 | Tau & | intrabody | | WO2014193632 SEQ ID NO: 5 | 22060 |
| | huntingtin | | | | |
| TAU1083 | Tau(pS422) | lg-kappa light | | WO2015197735 SEQ ID NO: 59 | 22061 |
| | | chain | | | |
| | | constant | | | |
| | | region | | | |
| TAU1084 | Tau(pS422) | 1g-kappa light | | WO2015197735 SEQ ID NO: 60 | 22062 |
| | | chain | | | |
| | | constant | | | |
| | | region | | | |
| TAU1085 | Tau | light chain | 1 A6 | WO2016137950 SEQ ID NO: 48 | 22063 |
| TAU1086 | Tau | light chain | 113F5-F7 | WO2016196726 SEQ ID NO: 91 | 22064 |
| TAU1087 | Tau | light chain | 11E10-B8 | WO2016196726 SEQ ID NO: 31 | 22065 |
| TAU1088 | Tau | light chain | 123E9-A1 | WO2016196726 SEQ ID NO: 141 | 22066 |
| TAU1089 | Tau | light chain | 125B11-H3 | WO2016196726 SEQ ID NO: 81 | 22067 |
| TAU1090 | Tau | light chain | 126F11-G11 | WO2016196726 SEQ ID NO: 181 | 22068 |
| TAU1091 | Tau | light chain | 12A10-E8 | WO2016196726 SEQ ID NO: 251 | 22069 |
| TAU1092 | Tau | light chain | 14F5-D9 | WO2016196726 SEQ ID NO: 211 | 22070 |
| TAU1093 | Tau | light chain | 15C6-A7 | WO2016196726 SEQ ID NO: 151 | 22071 |
| TAU1094 | Tau | light chain | 17H3.2 | WO2016112078 SEQ ID NO: 21 | 22072 |
| TAU1095 | Tau | light chain | 19F8-B1 | WO2016196726 SEQ ID NO: 161 | 22073 |
| TAU1096 | Tau | light chain | 19H6-F7 | WO2016196726 SEQ ID NO: 61 | 22074 |
| TAU1097 | Tau | light chain | 22G7-C9 | WO2016196726 SEQ ID NO: 231 | 22075 |
| TAU1098 | Tau | light chain | 24A11-D5 | WO2016196726 SEQ ID NO: 171 | 22076 |
| TAU1099 | Tau | light chain | 26C1-B11 | WO2016196726 SEQ ID NO: 101 | 22077 |
| TAU1100 | Tau | light chain | 26C1-C8 | WO2016196726 SEQ ID NO: 111 | 22078 |
| TAU1101 | Tau | Light chain | 29H2.10 | WO2016112078 SEQ ID NO: 24 | 22079 |
| TAU1102 | Tau | light chain | 30G1-B2 | WO2016196726 SEQ ID NO: 121 | 22080 |
| TAU1103 | Tau | light chain | 37D3-H9 | WO2016196726 SEQ ID NO: 11 | 22081 |
| TAU1104 | Tau | light chain | 37D3-H9b | WO2016196726 SEQ ID NO: 21 | 22082 |
| TAU1105 | Tau | light chain | 3A4-H4 | WO2016196726 SEQ ID NO: 51 | 22083 |
| TAU1106 | Tau | Light chain | 4G11 | WO2016137950 SEQ ID NO: 44 | 22084 |
| TAU1107 | Tau | light chain | 52F6-F11 | WO2016196726 SEQ ID NO: 271 | 22085 |
| TAU1108 | Tau | light chain | 54C1-H11 and | WO2016196726 SEQ ID NO: 41 | 22086 |
| | | | 61E7-C4 | | |
| TAU1109 | Tau | light chain | 55E7-F11 | WO2016196726 SEQ ID NO: 261 | 22087 |
| TAU1110 | Tau | light chain | 66F5-A1 | WO2016196726 SEQ ID NO: 131 | 22088 |
| TAU1111 | Tau | light chain | 73H6-B8 | WO2016196726 SEQ ID NO: 221 | 22089 |
| TAU1112 | Tau | light chain | 7A11-C12 | WO2016196726 SEQ ID NO: 241 | 22090 |
| TAU1113 | Tau | light chain | 89F4-A1 | WO2016196726 SEQ ID NO: 191 | 22091 |
| TAU1114 | Tau | light chain | 93A8-D2 | WO2016196726 SEQ ID NO: 201 | 22092 |
| TAU1115 | Tau | light chain | 94B2-C1 | WO2016196726 SEQ ID NO: 71 | 22093 |
| TAU1116 | Tau ps410 | light chain | hACl-36-3A8- | US20150175682 SEQ ID NO: 12 | 22094 |
| | | | Ab1 | | |
| TAU1117 | Tau | light chain | hu125B11- | WO2016196726 SEQ ID NO: 442 | 22095 |
| | | | H3.LC1 | | |
| TAU1118 | Tau | light chain | hu125B11- | WO2016196726 SEQ ID NO: 443 | 22096 |
| | | | H3.LC2 | | |
| TAU1119 | Tau | light chain | hu125B11- | WO2016196726 SEQ ID NO: 444 | 22097 |
| | | | H3.LC3 | | |
| TAU1120 | Tau | light chain | hu125B11- | WO2016196726 SEQ ID NO: 445 | 22098 |
| | | | H3.LC4 | | |
| TAU1121 | Tau | light chain | Hu37D3.v39 | WO2016196726 SEQ ID NO: 561 | 22099 |
| TAU1122 | Tau | light chain | Hu37D3.v40 | WO2016196726 SEQ ID NO: 571 | 22100 |
| TAU1123 | Tau | light chain | Hu37D3.v41 | WO2016196726 SEQ ID NO: 581 | 22101 |
| TAU1124 | Tau | light chain | Hu37D3-H9.v1 | WO2016196726 SEQ ID NO: 281 | 22102 |
| TAU1125 | Tau | light chain | Hu37D3- | WO2016196726 SEQ ID NO: 341 | 22103 |
| | | | H9.v28,A4 | | |
| TAU1126 | Tau | light chain | Hu37D3- | WO2016196726 SEQ ID NO: 349 | 22104 |
| | | | H9.v28.A4 | | |
| | | | IgG4- | | |
| | | | S228P.YTE | | |
| TAU1127 | Tau | light chain | Hu37D3-H9.v5 | WO2016196726 SEQ ID NO: 291 | 22105 |
| TAU1128 | Tau | light chain | Hu94B2.LC10 | WO2016196726 SEQ ID NO: 461 | 22106 |
| TAU1129 | Tau | light chain | Hu94B2.LC11 | WO2016196726 SEQ ID NO: 462 | 22107 |
| TAU1130 | Tau | light chain | Hu94B2.LC12 | WO2016196726 SEQ ID NO: 463 | 22108 |
| TAU1131 | Tau | light chain | Hu94B2.LC13 | WO2016196726 SEQ ID NO: 464 | 22109 |
| TAU1132 | Tau | light chain | Hu94B2.LC14 | WO2016196726 SEQ ID NO: 465 | 22110 |
| TAU1133 | Tau | light chain | Hu94B2.LC15 | WO2016196726 SEQ ID NO: 466 | 22111 |
| TAU1134 | Tau | light chain | Hu94B2.LC16 | WO2016196726 SEQ ID NO: 467 | 22112 |
| TAU1135 | Tau | light chain | Hu94B2.LC9 | WO2016196726 SEQ ID NO: 460 | 22113 |
| TAU1136 | Tau | light chain | Hu94B2.v105 | WO2016196726 SEQ ID NO: 301 | 22114 |
| TAU1137 | Tau(pS422) | light chain | MAb086 | WO2015197735 SEQ ID NO: 07 | 22115 |
| TAU1138 | Tau | light chain | | WO2016137811 SEQ ID NO: 1 | 22116 |
| TAU1139 | Tau | light chain | | WO2016137811 SEQ ID NO: 11 | 22117 |
| TAU1140 | Tau | light chain | | US8940272B2 SEQ ID NO: 11 | 22118 |
| TAU1141 | Tau ps411 | light chain | hACl-36-2B6- | US20150175682 SEQ ID NO: 13 | 22119 |
| | | | Abl | | |
| TAU1142 | Tau | light chain | DC8E8 | WO2016079597 SEQ ID NO: 8 | 22120 |
| | | variable | | | |
| | | domain | | | |
| TAU1143 | Tau | light chain | | WO2016137811 SEQ ID NO: 9 | 22121 |
| | | variable | | | |
| | | domain | | | |
| TAU1144 | Tau | single domain | Tau15 | WO2016055941 SEQ ID NO: 7 | 22122 |
| | | antibody | | | |
| TAU1145 | Tau | single domain | Tau81 | WO2016055941 SEQ ID NO: 8 | 22123 |
| | | antibody | | | |
| TAU1146 | pTau | Heavy chain | AB1 | WO2017005732A1 SEQ ID NO: 8 | 22124 |
| | (pS198/pS199/ | | | | |
| | pS202/pT205) | | | | |
| TAU1147 | pTau | Heavy chain | AB1 | WO2017005732A1 SEQ ID NO: | 22125 |
| | (pS198/pS199/ | | | 10 | |
| | pS202/pT205) | | | | |
| TAU1148 | pTau | Heavy chain | AB1 | WO2017005732A1 SEQ ID NO: | 22126 |
| | (pS198/pS199/ | | | 14 | |
| | pS202/pT205) | | | | |
| TAU1149 | pTau | Heavy chain | ABI | WO2017005732A1 SEQ ID NO: | 22127 |
| | (pS198/pS199/ | | | 15 | |
| | pS202/pT205) | | | | |
| TAU1150 | pTau | Heavy chain | AB1 | WO2017005732A1 SEQ ID NO: | 22128 |
| | (pS198/pS199/ | | | 16 | |
| | pS202/pT205) | | | | |
| TAU1151 | pTau | Heavy chain | AB1 | WO2017005732A1 SEQ ID NO: | 22129 |
| | (pS198/pS199/ | | | 20 | |
| | pS202/pT205) | | | | |
| TAU1152 | pTau | Heavy chain | AB1 | WO2017005732A1 SEQ ID NO: | 22130 |
| | (pS198/pS199/ | | | 21 | |
| | pS202/pT205) | | | | |
| TAU1153 | pTau | Heavy chain | AB1 | WO2017005732A1 SEQ ID NO: | 22131 |
| | (pS198/pS199/ | | | 22 | |
| | pS202/pT205) | | | | |
| TAU1154 | pTau | Heavy chain | AB1 | WO2017005732A1 SEQ ID NO: | 22132 |
| | (pS198/pS199/ | | | 23 | |
| | pS202/pT205) | | | | |
| TAU1155 | pTau | Heavy chain | AB1 | WO2017005732AI SEQ ID NO: | 22133 |
| | (pS198/pS199/ | | | 24 | |
| | pS202/pT205) | | | | |
| TAU1156 | pTau | Heavy chain | AB1 | WO2017005732A1 SEQ ID NO: | 22134 |
| | (pS198/pS199/ | | | 25 | |
| | pS202/pT205) | | | | |
| TAU1157 | Tau | Heavy chain | AB1 | WO2017005732A1 SEQ ID NO: | 22135 |
| | | | | 27 | |
| TAU1158 | pTAU (pS396) | Heavy Chain | C10.2 | US20170015738A1 SEQ ID NO: | 22136 |
| | | | | 16 | |
| TAU1159 | Tau | heavy chain | C2N-8E12 | WO2016201434A2 SEQ ID NO: | 22137 |
| | | | | 14 | |
| TAU1160 | pTAU (pS396) | Heavy Chain | C5.2 | US20170015738A1 SEQ ID NO: | 22138 |
| | | | | 24 | |
| TAU1161 | pTAU (pS396) | Heavy Chain | C8.3 | US20170015738A1 SEQ ID NO: | 22139 |
| | | | | 32 | |
| TAU1162 | pTAU (pS396) | Heavy Chain | D1.2 | US20170015738A1 SEQ ID NO: | 22140 |
| | | | | 8 | |
| TAU1163 | pTAU (pS396) | Heavy Chain | humanized C10.2 | US20170015738A1 SEQ ID NO: | 22141 |
| | | | | 35 | |
| TAU1164 | Tau | Heavy chain | mFab AB | WO2017005734A1 SEQ ID NO: | 22142 |
| | | | | 20 | |
| TAU1165 | Tau | Heavy chain | mFab AB1 | WO2017005734A1 SEQ ID NO: 8 | 22143 |
| TAU1166 | Tau | Heavy chain | | WO2017005734A1 SEQ ID NO: | 22144 |
| | | | | 10 | |
| TAU1167 | Tau | Heavy chain | | WO2017005734A1 SEQ ID NO: | 22145 |
| | | | | 11 | |
| TAU1168 | Tau | Heavy chain | | WO2017005734A1 SEQ ID NO: | 22146 |
| | | | | 12 | |
| TAU1169 | Tau | Heavy chain | | WO2017005734A1 SEQ ID NO: | 22147 |
| | | | | 13 | |
| TAU1170 | Tau | Heavy chain | | WO2017005734A1 SEQ ID NO: | 22148 |
| | | | | 22 | |
| TAU1171 | Tau | Heavy chain | | WO2017005734A1 SEQ ID NO: | 22149 |
| | | | | 23 | |
| TAU1172 | Tau | Heavy chain | | WO2017005734A1 SEQ ID NO: | 22150 |
| | | | | 54 | |
| TAU1173 | Tau | Heavy chain | | WO2017005734AI SEQ ID NO: | 22151 |
| | | | | 55 | |
| TAU1174 | Tau | Heavy chain | | WO2017005734A1 SEQ ID NO: | 22152 |
| | | | | 15 | |
| TAU1175 | Tau | Heavy chain | | WO2017005734A1 SEQ ID NO: | 22153 |
| | | | | 16 | |
| TAU1176 | Tau | Heavy chain | | WO2017005734A1 SEQ ID NO: | 22154 |
| | | | | 17 | |
| TAU1177 | Tau | Heavy chain | | WO2017005734AI SEQ ID NO: | 22155 |
| | | | | 18 | |
| TAU1178 | Tau | Heavy chain | C2N-8E12 | WO2016201434A2 SEQ ID NO: | 22156 |
| | | (VH1) | | 15 | |
| TAU1179 | Tau | Heavy chain | C2N-8E12 | WO2016201434A2 SEQ ID NO: | 22157 |
| | | (VH2) | | 16 | |
| TAU1180 | Tau | Heavy chain | C2N-8E12 | WO2016201434A2 SEQ ID NO: | 22158 |
| | | (VH3) | | 17 | |
| TAU1181 | Tau | Heavy chain | C2N-8E12 | WO2016201434A2 SEQ ID NO: | 22159 |
| | | (VH4) | | 18 | |
| TAU1182 | Tau (421) | Heavy chain | 1G10D2 | WO2017027685A2 SEQ ID NO: | 22160 |
| | | variable | | 12 | |
| | | domain | | | |
| TAU1183 | Tau (421) | Heavy chain | 1G11A10 | WO2017027685A2 SEQ ID NO: | 22161 |
| | | variable | | 20 | |
| | | domain | | | |
| TAU1184 | Tau pS404 | Heavy chain | 4E6G7 | WO2017027691A1 SEQ ID NO: | 22162 |
| | | variable | | 13 | |
| | | domain | | | |
| TAU1185 | Tau (421) | Heavy chain | 5G2A3 | WO2017027685A2 SEQ ID NO: | 22163 |
| | | variable | | 40 | |
| | | domain | | | |
| TAU1186 | Tau | Heavy chain | IPN001 VH | U.S. Pat. No. 9,567,395 SEQ ID NO: 18 | 22164 |
| | | variable | | | |
| | | region | | | |
| TAU1187 | Tau | Heavy chain | IPN002 VH | U.S. Pat. No. 9,56,7395 SEQ ID NO: 20 | 22165 |
| | | variable | | | |
| | | region | | | |
| TAU1188 | Tau | Heavy chain | AC1-35-1D2- | U.S. Pat. No. 9,540,434 SEQ ID NO: 86 | 22166 |
| | | variable | Ab1 | | |
| | | region (VH) | | | |
| TAU1189 | Tau | Heavy chain | AC1-35-2A1- | U.S. Pat. No. 9,540,434 SEQ ID NO: 109 | 22167 |
| | | variable | Abl, ACI-35~ | | |
| | | region (VH) | 2A1-Ab2, and | | |
| | | | ACI-35-4A6- | | |
| | | | Ab2 | | |
| TAU1190 | Tau | Heavy chain | ACI-35-2G5- | U.S. Pat. No. 9,540,434 SEQ ID NO: 111 | 22168 |
| | | variable | ABI | | |
| | | region (VH) | | | |
| TAU1191 | Tau | Heavy chain | ACI-35-2G5- | U.S. Pat. No. 9,540,434 SEQ ID NO: 113 | 22169 |
| | | variable | AB2 and ACI- | | |
| | | region (VH) | 35-2G5-AB3 | | |
| TAU1192 | Tau | Heavy chain | ACI-35-4A6- | U.S. Pat. No. 9,540,434 SEQ ID NO: 84 | 22170 |
| | | variable | Ab1 | | |
| | | region (VH) | | | |
| TAU1193 | Tau | Heavy chain | IPN002 VH | U.S. Pat. No. 9,567,395 SEQ ID NO: 28 | 22171 |
| | | variable | variant 1 | | |
| | | region, | | | |
| | | humanized | | | |
| TAU1194 | Tau | Heavy chain | IPN002 VH | U.S. Pat. No. 9,567,395 SEQ ID NO: 29 | 22172 |
| | | variable | variant 2 | | |
| | | region, | | | |
| | | humanized | | | |
| TAU1195 | Tau | Heavy chain | IPN002 VH | U.S. Pat. No. 9,567,395 SEQ ID NO: 30 | 22173 |
| | | variable | variant 3 | | |
| | | region, | | | |
| | | humanized | | | |
| TAU1196 | Tau | Heavy chain | IPN002 VH | U.S. Pat. No. 9,567,395 SEQ ID NO: 31 | 22174 |
| | | variable | variant 4 | | |
| | | region, | | | |
| | | humanized | | | |
| TAU1197 | Tau (pS422) | Heavy chain | VH35H5 | US20160376352A1 SEQ ID NO: | 22175 |
| | | variant 16 | | 65 | |
| TAU1198 | Tau | Heavy chain | C2N-8E12 | WO2016201434A2 SEQ ID NO: 1 | 22176 |
| | | variant gVH1 | | | |
| TAU1199 | Tau | Heavy chain | C2N-8E12 | WO2016201434A2 SEQ ID NO: 2 | 22177 |
| | | variant gVH2 | | | |
| TAU1200 | Tau | Heavy chain | C2N-8E12 | WO2016201434A2 SEQ ID NO: 3 | 22178 |
| | | variant gVH3 | | | |
| TAU1201 | Tau | Heavy chain | C2N-8E12 | WO2016201434A2 SEQ ID NO: 4 | 22179 |
| | | variant g VH4 | | | |
| TAU1202 | Tau (421) | Heavy Chain | 5B3C11 | WO2017027685A2 SEQ ID NO: | 22180 |
| | | VL2 Variable | | 32 | |
| | | Domain | | | |
| TAU1203 | Tau | Heavy chain; | | WO2017005734A1 SEQ ID NO: | 22181 |
| | | humanized | | 25 | |
| TAU1204 | Tau | Heavy chain; | | WO2017005734A1 SEQ ID NO: | 22182 |
| | | humanized | | 26 | |
| TAU1205 | Tau | Heavy chain; | | WO2017005734A1 SEQ ID NO: | 22183 |
| | | humanized | | 56 | |
| TAU1206 | Tau | Heavy chain; | | WO2017005734A1 SEQ ID NO: | 22184 |
| | | humanized | | 57 | |
| TAU1207 | Tau | Heavy chain; | | WO2017005732A1 SEQ ID NO: | 22185 |
| | | humanized | | 32 | |
| TAU1208 | Tau | Heavy chain; | | WO2017005732A1 SEQ ID NO: | 22186 |
| | | humanized | | 33 | |
| TAU1209 | Tau | Heavy chain; | | WO2017005732A1 SEQ ID NO: | 22187 |
| | | humanized | | 36 | |
| TAU1210 | Tau | Heavy chain; | | WO2017005732A1 SEQ ID NO: | 22188 |
| | | humanized | | 37 | |
| TAU1211 | Tau | Heavy chain; | | WO2017005732A1 SEQ ID NO: | 22189 |
| | | humanized | | 38 | |
| TAU1212 | Tau | Heavy chain; | | WO2017005732A1 SEQ ID NO: | 22190 |
| | | humanized | | 39 | |
| TAU1213 | pTau | Light chain | AB1 | WO2017005732A1 SEQ ID NO: 7 | 22191 |
| | (pS198/pS199/ | | | | |
| | pS202/pT205) | | | | |
| TAU1214 | pTau | Light chain | AB1 | WO2017005732A1 SEQ ID NO: 9 | 22192 |
| | (pS198/pS199/ | | | | |
| | pS202/pT205) | | | | |
| TAU1215 | pTau | Light chain | AB1 | WO2017005732A1 SEQ ID NO: | 22193 |
| | (pS198/pS199/ | | | 11 | |
| | pS202/pT205) | | | | |
| TAU1216 | pTau | Light chain | AB1 | WO2017005732A1 SEQ ID NO: | 22194 |
| | (pS198/pS199/ | | | 12 | |
| | S202/pT205) | | | | |
| TAU1217 | pTau | Light chain | AB1 | WO2017005732A1 SEQ ID NO: | 22195 |
| | (pS198/pS199/ | | | 13 | |
| | pS202/pT205) | | | | |
| TAU1218 | pTau | Light chain | AB1 | WO2017005732A1 SEQ ID NO: | 22196 |
| | (pS198/p$199/ | | | 17 | |
| | pS202/pT205) | | | | |
| TAU1219 | pTau | Light chain | AB1 | WO2017005732AI SEQ ID NO: | 22197 |
| | (pS198/pS199/ | | | 18 | |
| | pS202/pT205) | | | | |
| TAU1220 | pTau | Light chain | AB1 | WO2017005732A1 SEQ ID NO: | 22198 |
| | (pS198/pS199/ | | | 19 | |
| | pS202/pT205) | | | | |
| TAU1221 | Tau | Light chain | AB1 | WO2017005732A1 SEQ ID NO: | 22199 |
| | | | | 26 | |
| TAU1222 | pTAU (pS396) | Light Chain | C10.2 | US20170015738A1 SEQ ID NO: | 22200 |
| | | | | 15 | |
| TAU1223 | Tau | light chain | C2N-8E12 | WO2016201434A2 SEQ ID NO: 9 | 22201 |
| TAU1224 | pTAU (pS396) | Light Chain | C5.2 | US20170015738A1 SEQ ID NO: | 22202 |
| | | | | 23 | |
| TAU1225 | pTAU (pS396) | Light Chain | C8.3 | US20170015738A1 SEQ ID NO: | 22203 |
| | | | | 31 | |
| TAU1226 | pTAU (pS396) | Light Chain | D1.2 | US20170015738A1 SEQ ID NO: | 22204 |
| | | | | 7 | |
| TAU1227 | pTAU (pS396) | Light Chain | D1.2* | US20170015738A1 SEQ ID NO: | 22205 |
| | | | | 34 | |
| TAU1228 | pTAU (pS396) | Light Chain | humanized C10.2 | US20170015738A1 SEQ ID NO: | 22206 |
| | | | | 36 | |
| TAU1229 | Tau | Light chain | mFab AB 1 | WO2017005734A1 SEQ ID NO: | 22207 |
| | | | | 19 | |
| TAU1230 | Tau | Light chain | mFab AB1 | WO2017005734A1 SEQ ID NO: 7 | 22208 |
| TAU1231 | Tau | Light chain | | WO2017005734A1 SEQ ID NO: 9 | 22209 |
| TAU1232 | Tau | Light chain | | WO2017005734A1 SEQ ID NO: | 22210 |
| | | | | 14 | |
| TAU1233 | Tau | Light chain | | WO2017005734A1 SEQ ID NO: | 22211 |
| | | | | 21 | |
| TAU1234 | Tau | Light chain | C2N-8E12 | WO2016201434A2 SEQ ID NO: | 22212 |
| | | (VK1) | | 10 | |
| TAU1235 | Tau | Light chain | C2N-8E12 | WO2016201434A2 SEQ ID NO: | 22213 |
| | | (VK2) | | 11 | |
| TAU1236 | Tau | Light chain | C2N-8E12 | WO2016201434A2 SEQ ID NO: | 22214 |
| | | (VK3) | | 12 | |
| TAU1237 | Tau | Light chain | C2N-8E12 | WO2016201434A2 SEQ ID NO: | 22215 |
| | | (VK4) | | 13 | |
| TAU1238 | Tau (421) | Light Chain | 1G10D2 | WO2017027685A2 SEQ ID NO: 8 | 22216 |
| | | Variable | | | |
| | | Domain | | | |
| TAU1239 | Tau (421) | Light Chain | 1G11A10 | WO2017027685A2 SEQ ID NO: | 22217 |
| | | Variable | | 16 | |
| | | Domain | | | |
| TAU1240 | Tau pS404 | Light chain | 4E6G7 | WO2017027691A1 SEQ ID NO: 9 | 22218 |
| | | variable | | | |
| | | domain | | | |
| TAU1241 | Tau (421) | Light Chain | 5G2A3 | WO2017027685A2 SEQ ID NO: | 22219 |
| | | Variable | | 36 | |
| | | Domain | | | |
| TAU1242 | Tau | Light chain | IPN001 VL | U.S. Pat. No. 9,567,395 SEQ ID NO: 17 | 22220 |
| | | variable | | | |
| | | region | | | |
| TAU1243 | Tau | Light chain | IPN002 VL | U.S. Pat. No. 9,567,395 SEQ ID NO: 19 | 22221 |
| | | variable | | | |
| | | region | | | |
| TAU1244 | Tau | Light chain | ACI-35-1D2- | U.S. Pat. No. 9,540,434 SEQ ID NO: 87 | 22222 |
| | | variable | Ab1 | | |
| | | region (VK) | | | |
| TAU1245 | Tau | Light chain | ACI-35-2A1- | U.S. Pat. No. 9,540,434 SEQ ID NO: 117 | 22223 |
| | | variable | Ab1 | | |
| | | region (VK) | | | |
| TAU1246 | Tau | Light chain | ACI-35-2A1- | U.S. Pat. No. 9,540,434 SEQ ID NO: 110 | 22224 |
| | | variable | Ab2 | | |
| | | region (VK) | | | |
| TAU1247 | Tau | Light chain | ACI-35-2G5- | U.S. Pat. No. 9,540,434 SEQ ID NO: 112 | 22225 |
| | | variable | AB1 | | |
| | | region (VK) | | | |
| TAU1248 | Tau | Light chain | ACI-35-2G5- | U.S. Pat. No. 9,540,434 SEQ ID NO: 114 | 22226 |
| | | variable | AB2 and ACI- | | |
| | | region (VK) | 35-2G5-AB3 | | |
| TAU1249 | Tau | Light chain | ACI-35-4A6- | U.S. Pat. No. 9,540,434 SEQ ID NO: 85 | 22227 |
| | | variable | Abl | | |
| | | region (VK) | | | |
| TAU1250 | Tau | Light chain | ACI-35-4A6- | U.S. Pat. No. 9,540,434 SEQ ID NO: 119 | 22228 |
| | | variable | Ab2 | | |
| | | region (VK) | | | |
| TAU1251 | Tau | Light chain | IPN002 Vk | U.S. Pat. No. 9,567,395 SEQ ID NO: 32 | 22229 |
| | | variable | variant 1 | | |
| | | region, | | | |
| | | humanized | | | |
| TAU1252 | Tau | Light chain | IPN002 Vk | U.S. Pat. No. 9,567,395 SEQ ID NO: 33 | 22230 |
| | | variable | variant 2 | | |
| | | region, | | | |
| | | humanized | | | |
| TAU1253 | Tau | Light chain | IPN002 Vk | U.S. Pat. No. 9,567,395 SEQ ID NO: 34 | 22231 |
| | | variable | variant 3 | | |
| | | region, | | | |
| | | humanized | | | |
| TAU1254 | Tau | Light chain | IPN002 Vk | U.S. Pat. No. 9,567,395 SEQ ID NO: 35 | 22232 |
| | | variable | variant 4 | | |
| | | region, | | | |
| | | humanized | | | |
| TAU1255 | Tau (pS422) | Light chain | VL31A1 | US20160376352A1 SEQ ID NO: | 22233 |
| | | variant 18 | | 66 | |
| TAU1256 | Tau (pS422) | Light chain | VL49G1 | US20160376352A1 SEQ ID NO: | 22234 |
| | | variant 19 | | 67 | |
| TAU1257 | Tau (pS422) | Light chain | VL35F2 | US20160376352A1 SEQ ID NO: | 22235 |
| | | variant 20 | | 68 | |
| TAU1258 | Tau (pS422) | Light chain | VL53A2 | US20160376352A1 SEQ ID NO: | 22236 |
| | | variant 21 | | 69 | |
| TAU1259 | Tau (pS422) | Light chain | VL35G4 | US20160376352A1 SEQ ID NO: | 22237 |
| | | variant 22 | | 78 | |
| TAU1260 | Tau (p$422) | Light chain | VL4G1 | US20160376352A1 SEQ ID NO: | 22238 |
| | | variant 24 | | 86 | |
| TAU1261 | Tau | Light chain | C2N-8E12 | WO2016201434A2 SEQ ID NO: 5 | 22239 |
| | | variant gVL1 | | | |
| TAU1262 | Tau | Light chain | C2N-8E12 | WO2016201434A2 SEQ ID NO: 6 | 22240 |
| | | variant gVL2 | | | |
| TAU1263 | Tau | Light chain | C2N-8E12 | WO2016201434A2 SEQ ID NO: 7 | 22241 |
| | | variant gVL3 | | | |
| TAU1264 | Tau | Light chain | C2N-8E12 | WO2016201434A2 SEQ ID NO: 8 | 22242 |
| | | variant gVL4 | | | |
| TAU1265 | Tau (421) | Light chain | 5B3C11 | WO2017027685A2 SEQ ID NO: | 22243 |
| | | VL1 variable | | 24 | |
| | | domain | | | |
| TAU1266 | Tau (421) | Light chain | 5B3C11 | WO2017027685A2 SEQ ID NO: | 22244 |
| | | VL2 variable | | 28 | |
| | | domain | | | |
| TAU1267 | Tau | Light chain; | | WO2017005734A1 SEQ ID NO: | 22245 |
| | | humanized | | 24 | |
| TAU1268 | Tau | Light chain; | | WO2017005732A1 SEQ ID NO: | 22246 |
| | | humanized | | 30 | |
| TAU1269 | Tau | Light chain; | | WO2017005732A1 SEQ ID NO: | 22247 |
| | | humanized | | 31 | |
| TAU1270 | Tau | Light chain; | | WO2017005732A1 SEQ ID NO: | 22248 |
| | | humanized | | 34 | |
| TAU1271 | Tau | Light chain; | | WO2017005732A1 SEQ ID NO: | 22249 |
| | | humanized | | 35 | |
| TAU1272 | Tau (421) | scFv | 1G10D2 | WO2017027685A2 SEQ ID NO: | 22250 |
| | | | | 47 | |
| TAU1273 | Tau (421) | scFv | 1G10D2 | WO2017027685A2 SEQ ID NO: | 22251 |
| | | | | 48 | |
| TAU1274 | Tau (421) | scFv | 1G10D2 | WO2017027685A2 SEQ ID NO: | 22252 |
| | | | | 49 | |
| TAU1275 | Tau (421) | scFv | 1G10D2 | WO2017027685A2 SEQ ID NO: | 22253 |
| | | | | 50 | |
| TAU1276 | Tau (421) | scFv | 1G10D2 | WO2017027685A2 SEQ ID NO: | 22254 |
| | | | | 51 | |
| TAU1277 | Tau (421) | scFv | 1G10D2 | WO2017027685A2 SEQ ID NO: | 22255 |
| | | | | 52 | |
| TAU1278 | Tau (421) | scFv | 1G10D2 | WO2017027685A2 SEQ ID NO: | 22256 |
| | | | | 53 | |
| TAU1279 | Tau (421) | scFv | 1G10D2 | WO2017027685A2 SEQ ID NO: | 22257 |
| | | | | 54 | |
| TAU1280 | Tau (421) | scFv | 1G10D2 | WO2017027685A2 SEQ ID NO: | 22258 |
| | | | | 55 | |
| TAU1281 | Tau (421) | scFv | 1G10D2 | WO2017027685A2 SEQ ID NO: | 22259 |
| | | | | 56 | |
| TAU1282 | Tau (421) | scFv | 1G11A10 | WO2017027685A2 SEQ ID NO: | 22260 |
| | | | | 57 | |
| TAU1283 | Tau (421) | scFv | 1G11A10 | WO2017027685A2 SEQ ID NO: | 22261 |
| | | | | 58 | |
| TAU1284 | Tau (421) | scFv | 1G11A10 | WO2017027685A2 SEQ ID NO: | 22262 |
| | | | | 59 | |
| TAU1285 | Tau (421) | scFv | 1G11A10 | WO2017027685A2 SEQ ID NO: | 22263 |
| | | | | 60 | |
| TAU1286 | Tau (421) | scFv | 1G11A10 | WO2017027685A2 SEQ ID NO: | 22264 |
| | | | | 61 | |
| TAU1287 | Tau (421) | scFv | 1G11A10 | WO2017027685A2 SEQ ID NO: | 22265 |
| | | | | 62 | |
| TAU1288 | Tau (421) | scFv | 1G11A10 | WO2017027685A2 SEQ ID NO: | 22266 |
| | | | | 63 | |
| TAU1289 | Tau (421) | scFv | 1G11A10 | WO2017027685A2 SEQ ID NO: | 22267 |
| | | | | 64 | |
| TAU1290 | Tau (421) | scFv | 1G11A10 | WO2017027685A2 SEQ ID NO: | 22268 |
| | | | | 65 | |
| TAU1291 | Tau (421) | scFv | 1G11A10 | WO2017027685A2 SEQ ID NO: | 22269 |
| | | | | 66 | |
| TAU1292 | Tau pS404 | scFv | 4E6G7 | WO2017027691A1 SEQ ID NO: | 22270 |
| | | | | 17 | |
| TAU1293 | Tau (421) | scFv | 5B3C11 (VL1) | WO2017027685A2 SEQ ID NO: | 22271 |
| | | | | 67 | |
| TAU1294 | Tau (421) | scFv | 5B3C11 (VL1) | WO2017027685A2 SEQ ID NO: | 22272 |
| | | | | 68 | |
| TAU1295 | Tau (421) | scFv | 5B3C11 (VL1) | WO2017027685A2 SEQ ID NO: | 22273 |
| | | | | 59 | |
| TAU1296 | Tau (421) | scFv | 5B3C11 (VL1) | WO2017027685A2 SEQ ID NO: | 22274 |
| | | | | 70 | |
| TAU1297 | Tau (421) | scFv | 5B3C11 (VL1) | WO2017027685A2 SEQ ID NO: | 22275 |
| | | | | 71 | |
| TAU1298 | Tau (421) | scFv | 5B3C11 (VL1) | WO2017027685A2 SEQ ID NO: | 22276 |
| | | | | 72 | |
| TAU1299 | Tau (421) | scFv | 5B3C11 (VL1) | WO2017027685A2 SEQ ID NO: | 22277 |
| | | | | 73 | |
| TAU1300 | Tau (421) | scFv | 5B3C11 (VL1) | WO2017027685A2 SEQ ID NO: | 22278 |
| | | | | 74 | |
| TAU1301 | Tau (421) | scFv | 5B3C11 (VL1) | WO2017027685A2 SEQ ID NO: | 22279 |
| | | | | 75 | |
| TAU1302 | Tau (421) | scFv | 5B3C11 (VL1) | WO2017027685A2 SEQ ID NO: | 22280 |
| | | | | 76 | |
| TAU1303 | Tau (421) | scFy | 5B3C11 (VL2) | WO2017027685A2 SEQ ID NO: | 22281 |
| | | | | 77 | |
| TAU1304 | Tau (421) | scFv | 5B3C11 (VL2) | WO2017027685A2 SEQ ID NO: | 22282 |
| | | | | 78 | |
| TAU1305 | Tau (421) | scFv | 5B3C11 (VL2) | WO2017027685A2 SEQ ID NO: | 22283 |
| | | | | 79 | |
| TAU1306 | Tau (421) | scFv | 5B3C11 (VL2) | WO2017027685A2 SEQ ID NO: | 22284 |
| | | | | 80 | |
| TAU1307 | Tau (421) | scFv | 5B3C11 (VL2) | WO2017027685A2 SEQ ID NO: | 22285 |
| | | | | 81 | |
| TAU1308 | Tau (421) | scFv | 5B3C11 (VL2) | WO2017027685A2 SEQ ID NO: | 22286 |
| | | | | 82 | |
| TAU1309 | Tau (421) | scFv | 5B3C11 (VL2) | WO2017027685A2 SEQ ID NO: | 22287 |
| | | | | 83 | |
| TAU1310 | Tau (421) | scFv | 5B3C11 (VL2) | WO2017027685A2 SEQ ID NO: | 22288 |
| | | | | 84 | |
| TAU1311 | Tau (421) | scFv | 5B3C11 (VL2) | WO2017027685A2 SEQ ID NO: | 22289 |
| | | | | 85 | |
| TAU1312 | Tau (421) | sc.Fv | 5B3C11 (VL2) | WO2017027685A2 SEQ ID NO: | 22290 |
| | | | | 86 | |
| TAU1313 | Tau (421) | scFv | 5G2A3 | WO2017027685A2 SEQ ID NO: | 22291 |
| | | | | 87 | |
| TAU1314 | Tau (421) | scFv | 5G2A3 | WO2017027685A2 SEQ ID NO: | 22292 |
| | | | | 88 | |
| TAU1315 | Tau (421) | scFv | 5G2A3 | WO2017027685A2 SEQ ID NO: | 22293 |
| | | | | 89 | |
| TAU1316 | Tau (421) | sc.Fv | 5G2A3 | WO2017027685A2 SEQ ID NO: | 22294 |
| | | | | 90 | |
| TAU1317 | Tau (421) | scFv | 5G2A3 | WO2017027685A2 SEQ ID NO: | 22295 |
| | | | | 91 | |
| TAU1318 | Tau (421) | scFv | 5G2A3 | WO2017027685A2 SEQ ID NO: | 22296 |
| | | | | 92 | |
| TAU1319 | Tau (421) | scFv | 5G2A3 | WO2017027685A2 SEQ ID NO: | 22297 |
| | | | | 93 | |
| TAU1320 | Tau (421) | scFv | 5G2A3 | WO2017027685A2 SEQ ID NO: | 22298 |
| | | | | 94 | |
| TAU1321 | Tau (421) | scFv | 5G2A3 | WO2017027685A2 SEQ ID NO: | 22299 |
| | | | | 95 | |
| TAU1322 | Tau (421) | scFv | 5G2A3 | WO2017027685A2 SEQ ID NO: | 22300 |
| | | | | 96 | |

Payload regions of the viral genomes of the disclosure may encode any anti-tau antibodies, or tau-associated antibodies, not limited to those described in Table 13, including antibodies that are known in the art and/or antibodies that are commercially available. This may include fragments of such antibodies or antibodies that have been developed to comprise one or more of such fragments (e.g., variable domains or complementarity determining regions (CDRs)). Anti-tau antibodies that may be encoded by payloads of the disclosure include, but are not limited to, AT8 (pSer²⁰²/pThr²⁰²: ThermoFisher, Waltham, MA; described in International Publication No, WO1995017429, the contents of which are herein incorporated in their entirety), AT100 (pSer²¹²/pSer²¹⁴; ThermoFisher, Waltham, MA; described in U.S. Pat. No. 6,121,003, the contents of which are herein incorporated in their entirety), AT180 (pThr²³¹; ThermoFisher, Waltham, MA; described in International Publication No. WO1995017429, the contents of which are herein incorporated by reference in their entirety), MC-1 (Tau^{2-18/31 2-342} conformational antibody; as described in international Publication WO199620218, the contents of which are herein incorporated by reference in their entirety), MC-6 (pSer²³⁵; described in U.S. Pat. No. 5,811,310, the contents of which are herein incorporated in their entirety), TG-3 (pThr²³¹; described in Jicha, G A et al., 1997 J Neurochem 69(5):2087-95, the contents of which are herein incorporated by reference in their entirety), CP13 (pSer²⁰²), CP27 (human Tau¹³⁰⁻¹⁵⁰), Tau12 (human Tau⁹⁻¹⁸; Abcam, Cambridge, MA), TG5 (Tau²⁰²; described in U.S. Pat. No. 5,811,310), described in U.S. Pat. No. 5,811,310), PHF-1 (pSer³⁹⁶/pSer⁴⁰⁴ described in International Publication WO199620218), Alz50 (Tau⁷⁻⁹ and Tau³¹²⁻³⁴² conformational epitope; described in U.S. Pat. No. 5,811,310 and Carmel, G et. al. 1996 J Biol Chem 271(51):32780-32795 and Jicha, G A et al. 1997 J Neurosci Res 48(2):128-132, the contents of each of which are herein incorporated by reference in their entirety), Tau-1 (de-phosphorylated Ser¹⁹⁵/Ser¹⁹⁸/Ser¹⁹⁹/Ser²⁰²; ThermoFisher Waltham, MA), Tau46 Abcam, Cambridge, MA), pS199 (ThermoFisher, Waltham, MA), pT205, pS396 (ThermoFisher, Waltham, MA; described in U.S. Pat. No. 8,647,631, the contents of which are herein incorporated by reference in their entirety), pS404 (ThermoFisher, Waltham, MA; described in U.S. Pat. No. 8,647,631, the contents of which are herein incorporated by reference in their entirety), pS422 (ThermoFisher, Waltham, MA), A0024 (hTau²⁴³⁻⁴⁴¹ Dako, Glostrup, Denmark), HT7 (hTau¹⁵⁹⁻¹⁶³; ThermoFisher, Waltham, MA), Tau2 (hTau⁵²⁻⁶⁸, Abcam, Cambridge, MA), AD2 (pSer³⁹⁶/pSer⁴⁰⁴, Bio-Rad Laboratories, Hercules, CA), AT120 (hTau²¹⁶⁻²²⁴; described in U.S. Pat. No. 5,843,779, the contents of which are herein incorporated by reference in their entirety), AT270 (pThr¹⁸¹; ThermoFisher, Waltham, MA), 12E8 (pSer²⁶² and/or Ser 356); K9JA (hTau 243-441; Dako, Caprinteria, CA), TauC3 (hTau Asp441; Santa Cruz Biotechnology, Dallas, TX; described in United States Patent Publication US20120244174 and Gamblin, T C et al 2003 PNAS 100(17):10032-7, the contents of each of which are herein incorporated by reference in their entirety), 4E6G7 (pSer³⁹⁶/pSer⁴⁰⁴; described in United States Patent Publication No. US2010316564 and Congdon, E. E. et al., 2016. Molecular Neurodegeneration August 30; 11(1):62, the contents of which are herein incorporated by reference in their entirety), 6B2 and variants thereof, described in International Patent Publication WO2016007414, the contents of which are herein incorporated by reference in their entirety, RZ3 (pThr²³¹), PG5 (pSer⁴⁰⁹), BT2 (pS^{199/202}), DA31 (Tau 150190), CP9 (pThr²³¹) Ta1505 (phospho site between Tau^{410/421}, particularly pSer⁴¹³ as described in United States Patent Publication US20150183854 and Umeda, T. et al., 2015. Ann Clin Trans Neurol 2(3): 241-255, the contents of each of which are herein incorporated by reference in their entirety), PHF-6 (pThr²³⁷, as described in Hoffman R et. al., 1997, Biochemistry 36; 8114-8124, the contents of which are herein incorporated by reference in their entirety), PHF-13 (pSer³⁹⁶, as described in Hoffman R et. al., 1997. Biochemistry 36; 8114-8124); 16B5 (Tau 25-46, as described in United States Publication US20160031976, the contents of which are herein incorporated by reference in their entirety), DC8E8 (as described in United States Patent Publication US20150050215, the contents of which are herein incorporated by reference in their entirety), PT1 or PT3 (as described in U.S. Pat. No. 9,371,376, the contents of which are herein incorporated by reference in their entirety), 4G11 (Tau⁵⁷⁻⁶⁴, as described in International Publication WO2016137950, the contents of which are herein incorporated by reference in their entirety), 1A6 (Tau⁷⁻¹⁷ and Tau²¹⁵⁻²²⁰, as described in International Publication WO2016137950), Tau15 or Tau81 (as described in International Publication WO2016055941, the contents of which are herein incorporated by reference in their entirety), TOC-1 (dimerized or aggregated tau, as described in International Publication WO2012149365, the contents of which are herein incorporated by reference in their entirety), pS404IgG2a/k (Neotope Biosciences, South San Francisco, CA; as described in Ittner et al., 2015. Neurochemistry 132:135145, the contents of which are herein incorporated by reference in their entirety), TOMA. (tau oligomer monoclonal antibody; as described in U.S. Pat. Nos. 8,778,343 and 9,125,846; International Publications WO2012051498 and WO2011026031, or United States Publication Nos. US20150004169 and US20150322143, and Castillo-Carranza, D L et al., 2014 J Neurosci 34(12)426072, the contents of each of which are herein incorporated by reference in their entirety), TTC-99 (oligomeric tau), BMS-986168 (as described in United States Patent Publication US2014294831, international Publication WO2015081085 and U.S. Pat. No. 8,980,271, the contents of which are herein incorporated by reference in their entirety), 3H3 (pan-amyloid epitope; described in Levites, et al 2015 J Neurosci 35(16)6265-76, the contents of which are herein incorporated by reference in their entirety), cis-pT231 (described in International Publications WO2012149334 and WO2011056561, the contents of which are herein incorporated by reference in their entirety), CP-3 (pSer²¹⁴; described in Jicha et al 1999 J Neurosci 19(17):7486-94, the contents of which are herein incorporated by reference in their entirety), INT1 (Tau²⁻¹⁸; as described in United States Patent Publication 20160031978, the contents of which are herein incorporated by reference in their entirety), Tau-nY29 (nTyr²⁹; described in Reynolds M R, et al., 2006 J Neurosci 26(42):10636-45, the contents of which are herein incorporated by reference in their entirety), Tau-nY197 (nTyr¹⁹⁷; described in Reyes, J F et al., 2012 Acta Neuropathol 123(1):119-32, the contents of which are herein incorporated by reference in their entirety), Tau-nY394 (nTyr³⁹⁴; described in Reyes, J F et al 2012), 4E4 (Tau³³⁷⁻³⁴³ Tau 387-397; described in International Publication WO2012049570 and United States Patent Publication US20150252102, the contents of each of which are herein incorporated by reference in their entirety), ADx210 (described in United States Patent Publication US20140161875, the contents of which are herein incorporated by reference in their entirety), ADx215 (described in United States Patent Publication US20140161875), ADx202 (as described in International Publication WO2015004163, the contents of which are herein incorporated by reference in their entirety), AP422 (pSer⁴²² described in Hasegawa, M et al 1996 FEBS Lett 384:25-30, the contents of which are herein incorporated by reference in their entirety), Tau5 (Tau²¹⁰⁻²⁴¹), RTA2 (Tau²⁷³⁻²⁸³), RTAC (Tau⁴²⁶⁻⁴⁴¹), RTA1 (Tau²⁵⁷⁻²⁷⁴), T46 (Tau³⁹⁵⁻⁴³²), T49, MIGT4, O.BG.15, 525, 3-39, 4F1, MapTau (Tau⁹⁵⁻¹⁰⁸; SMI Covance), T1, HYB33801 (Tau 5-12), Tau13 (Tau t-r8), B11E8, 5J20 (14-3-3 tau), DC25 (71Tau 347-353), DC39N11 (Tau⁴⁵⁻⁷³), DC-11 (Tau³²¹⁻³⁹¹; described in U.S. Pat. No. 7,746,180, the contents of which are herein incorporated by reference in their entirety), DC39 (Tau⁴⁰¹⁻⁴¹¹), DC4R, n847 (nitrated tau), SPM452, T14, 1E1/A6 (Tau²⁷⁵⁻²⁹¹), 5E2, 8E6/C11 (Tau²⁰⁹⁻²²⁴), 2E12 (pT231), NFT200, 248E5 (Tau 3-14), IG2 (Thr¹⁷⁵, Thr¹⁸¹, Thr²³¹; as described in International Publication WO2016041553, the contents of which are herein incorporated by reference in their entirety), YP3 (as described in WO2007019273, the contents of which are herein incorporated by reference in their entirety), YP4 (as described in WO2007019273) and 14-3-3 Tau (pSer14-3-3 binding motif; Abcam, Cambridge, MA). Further, anti-tau antibodies may be any of those listed in the antibody section of Alzforum.org or at the Antibody Resource Page.com, the contents of each of which are herein incorporated by reference in their entirety. Further, anti-tau antibodies may be any commercially available anti-tau antibody. Additional antibodies may include any of those taught in Petry, F. R. et al., 2014. PLoS One 9(5): e94251, the contents of which are herein incorporated by reference in their entirety. In one example, such antibodies may include any of those described in Jicha, G. A. et 1997. Journal of Neuroscience Research 48:128-132, the contents of which are herein incorporated by reference in their entirety. One such antibody, MC-1, recognizes distinct conformations of tau that are associated with neurological disease.

In some embodiments, the viral particles may have a payload region comprising any of the anti-tau antibodies as described in international Publication WO2017189963, the contents of which are herein incorporated by reference in their entirety. As a non-limiting example, the payload region may comprise one or more of the anti-tau antibodies as described in Table 13 of International Publication WO2017189963. In some embodiments, the payload region encodes one or more anti-tau antibodies selected from SEC) ID NO: 2948-4269 as described in WO2017189963.

In some embodiments, payloads may encode anti-tau antibodies (or fragments thereof) taught in United States Publication No. US2014294831, the contents of which are herein incorporated by reference in their entirety. Such antibodies may include IPN001 and/or IPN002 antibodies or fragments of such antibodies. In some cases, variable domains of IPN002 as presented in FIGS. 2A and 2B of US2014294831 may be used (e.g., incorporated into another antibody). In some cases, CDR regions of IPN002 as underlined in FIGS. 2A and 2B may be used (e.g., incorporated into another antibody or used to prepare humanized versions of IPN002). In some cases, anti-tau antibodies may include any of the IPN001 or IPN002 antibody variants taught in US2014294831 (e.g., in FIGS. 9-16 of that publication). In some embodiments, this antibody is also referred to as BMS-986168.

In some cases, payloads may encode anti-tau antibodies (or fragments thereof) taught in Otvos, L. et al., 1994, J Neurosci. Res 39(6):669-73, the contents of which are herein incorporated by reference in their entirety. Such antibodies may include monoclonal antibody PHF-1 or fragments thereof. The PIF-1 antibody binds to tau paired helical filaments, a pathological conformation of tau, found in certain neurological disorders, including Alzheimer's disease. Further, antibody affinity is increased when either serine 396 or serine 404 of tau is phosphorylated and even further increased when both are phosphorylated.

In some embodiments, payloads may encode anti-tau antibodies (or fragments thereof) taught in U.S. Pat. No. 5,811,310, the contents of which are herein incorporated by reference in their entirety. Such embodiments may include monoclonal antibodies PHF-1 or MC-1 or fragments thereof. MC-1 is a conformational antibody binding to the epitopes presented in Jicha, CI. A., et al., 1997. J Neurosci Res 48(128-132).

In some embodiments, payloads may encode anti-tau antibodies (or fragments thereof) taught in International Publication Number WO2015035190, the contents of which are herein incorporated by reference in their entirety. Such embodiments may include, but are not limited to, antibodies PHF-1 or MC-1 or fragments thereof. Viral genomes of the viral particles of the present disclosure may comprise or encode any of SEQ ID NO: 1-6 of WO2015035190.

Anti-tau antibodies (or fragments thereof) encoded by viral genomes of the disclosure may include antibodies that bind to one or more of the epitopes presented in Otvos, L. et al., 1994. J Neurosci, Res 39(6):669-73 (e.g., any of those presented in Table 1 of that publication).

In some embodiments, payloads may encode anti-tau antibodies (or fragments thereof) taught in U.S. Pat. No. 7,746,180, the contents of which are herein incorporated by reference in their entirety. Such embodiments may include antibody DC-11 or fragments thereof.

In some embodiments, the antibodies encoded by the viral genomes of the present disclosure may target any of the antigenic regions or epitopes described in United States Patent Publication No US2008050383 or US20100316564, the contents of which are herein incorporated by reference in their entirety. In some embodiments, the antibody targets pS396/pS404. Such embodiments may include antibody 4E6 and/or variants or fragments thereof. The affinity of antibody 4E6 for soluble PHF and its ability to reduce soluble phospho tau has been described in Congdon, E. E. et al., 2016. Molecular Neurodegeneration August 30; 11(1):62, the contents of which are herein incorporated by reference in their entirety.

In some embodiments, the antibodies encoded by the viral genomes of the present disclosure may target any of the antigenic regions or epitopes described in International Patent Publication WO1998022120, the contents of which are herein incorporated by reference in their entirety. In some embodiments, the antibody may be PHF-6 (pT231), or fragments or variants thereof. In another embodiment, the antibody may be PHF-13 (pS396), or a fragment of variant thereof. These antibodies are further described in Hoffman et al., 1997. Biochemistry 36: 8114-8124, the contents of which are herein incorporated by reference in their entirety.

In some embodiments, the antibodies encoded by the viral genomes of the present disclosure may target any of the antigenic regions or epitopes described in International Publication WO2016126993, the contents of which are herein incorporated by reference in their entirety. The antibodies may be derived from any of the tau epitopes described in Table A of WO2016126993. In some embodiments, the antibody of the present disclosure may comprise any of the sequences listed in Table B or Table 1 of WO2016126993.

In some embodiments, the antibodies encoded by the viral genomes of the present disclosure may target any of the antigenic regions or epitopes described in United States Patent Publication US20120244174, the contents of which are herein incorporated by reference in their entirety. In some embodiments, the antibody may bind to caspase-cleaved tau. In some embodiments, the epitope for antibodies targeting caspase cleaved tau is aspartic acid 421. In another embodiment, the epitope for antibodies targeting caspase cleaved tau may be the C-terminus after glutamic residue Glu391. In yet another embodiment, the epitope for antibodies targeting caspase cleaved tau may be at the N-terminus at aspartic acid residue 13. In another embodiment, the antibody may be TauC3.

In some embodiments, the antibodies encoded by the viral genomes of the present disclosure may target any of the antigenic regions or epitopes described in United States Patent Publication US20160031978, the contents of which are herein incorporated by reference in their entirety. In some embodiments, the antibody may bind to tau N-terminal residues associated with the PP1/GSK3 signaling cascade. In some embodiments, the antibody may be TNT1.

In some embodiments, the antibodies encoded by the viral genomes of the present disclosure may be any of those described in d'Abramo, C et al., 2015. PLOS One 10(8):e0135774, the contents of which are herein incorporated by reference in their entirety. In some embodiments, the antibody may be CP13 (pS202), or a fragment or variant thereof. In another embodiment, the antibody may be RZ3 (pT231), or a fragment or variant thereof. In another embodiment, the antibody may be PG-5 (pS409), or a fragment or variant thereof.

Anti-tau antibodies or fragments thereof encoded by the viral genomes of the present disclosure may target tau in any antigenic form. As non-limiting examples, antigenic tau may be an unphosphorylated or unmodified tau protein, a phosphorylated or otherwise post-translationally modified tau protein (O-GlnAcylated, or nitrosylated), an oligomeric species of tau protein, a soluble species of tau protein, an insoluble species of tau protein, a conformationally abnormal species of tau protein, a neuropathological form of tau protein and/or a neurofibrillary tangle or a precursor thereof.

Anti-tau antibodies or fragments thereof encoded by the viral genomes of the disclosure, may target any antigenic region or epitope along the full length of any of the six human tau protein isoforms. As non-limiting examples, the targeted antigenic peptides of the tau protein may be any of the following phosphorylated sites pT50, pS396, pS396-pS404, pS404, pS396-pS404-pS422, pS409, pS413, pS422, pS198, pS199, P5199-0202,0202, pT205, p717212, pS214, pT212-pS214, p717181, pT231, cis-pT231, pS235, pS238, pT245, 135262, pY310, pY394, pY324, pY356, pTau¹⁷⁷⁻¹⁸⁷, pY610, pS622, nitrosylated tau (nY18, nY29), methylated tau (di-meK281, dimeK311), O-GlnAcylated tau at 5400, any of the following acetylated sites acK174, acK274, acK280, acK281 and/or any combination thereof. Acetylated tau proteins and associated antigenic peptides are described in Min et al., 2010, Neuron., 67, 953-966, Min et al., 2015, Nature Medicine., 10, 1154-1162, Cohen et al., 2011, Nature Communications., 2, 252, Gorsky et al., 2016, Scientific Report., 6, 22685, Tracy et al. 2016, Neuron., 90, 245-260, the contents of each of which are herein incorporated by reference in their entirety. Phosphorylated tau proteins and associated antigenic peptides are described in Asuni et al., 2007, Jr Neurosci., 27, 9115-9129, Boutajangout et al., 2010, 0.1 Neurosci., 30, 16559-16566, Boutajangout et al., 2011, J Neurochem., 118, 658-667, Chai et al., 2011, Jr Biol Chem., 286, 34457-34467, Gu et al., 2011, J Bio Chem., 288, 33081-33095, Sankaranarayanan et al., 2015, PloS One, 10, e0125614, Ittner et al., 2015, J Neurochem., 132, 135-145, D'Abramo et al., 2016, Neurobiol Aging., 37, 58-65, Collin et al. 2014, Brain., 137, 2834-2846, Kondo et al., 2015, Nature., 523, 431-436, the contents of each of which are herein incorporated by reference in their entirety.

In some embodiments, the antibody encoded by the viral genomes of the present disclosure may be a pS409 targeting antibody as described in Lee et al., 2016, Cell Reports, 16, 1690-1700, or International Patent Publication WO2013151762, the contents of each of which are herein incorporated by reference in their entirety. In some embodiments, this antibody may be RG6100 or 8071057 or variants or fragments thereof.

In some embodiments, the antibody encoded by the viral genomes of the present disclosure may be a pS413 targeting antibody as described in Umeda et al., 2015, Ann Clin Trans Neurol., 2(3), 241-255 or International Patent Publication WO2013180238, the contents of each of which are herein incorporated by reference in their entirety. In some embodiments, the antibody is Ta1505 or variants or fragments thereof.

In some embodiments, the antibody encoded by the viral genomes of the present disclosure may target a tau epitope with amino acid residues 210-275, more specifically pS238 and/or pT245, as described in International Publication WO2011053565, the contents of which are herein incorporated by reference in their entirety.

In some embodiments, the CDRs of an antibody encoded by the viral genomes of the present disclosure may be any of those listed in or incorporated in the antibody sequences of Table 13. In some embodiments, the CDRs may be any of those described in International Publication WO2015122922, the contents of which are herein incorporated by reference in their entirety. In some embodiments, a CDR may be any of those chosen from the group of SEQ 11 NO: 41, 49, or 57 of WO2015122922. Further a CDR of an antibody encoded by the viral genomes of the present disclosure may have 50%, 60%, 70%, 80%, 90%, or 95% identity to SEQ. ID NO: 41, 49, or 57 of WO2015122922.

In some embodiments, the antibodies encoded by the viral genomes of the present disclosure may be any of those described in International Publication WO2016097315, the contents of which are herein incorporated by reference in their entirety. In some embodiments, the antibody may have an amino acid sequence as shown by SEQ ID NO: 2, 11, 20, 29, 38, 47, 56, 65, 74, 83, 92, 101, 110, 119, 128, 137, 146, 155, 164, 173, 182, 191, 209, 218, 226, or 227 of WO2016097315.

In some embodiments, the antibodies encoded by the viral genomes of the present disclosure may be a multispecific blood brain barrier receptor antibody that also targets tau, as described in International Publication WO2016094566, the contents of which are herein incorporated by reference in their entirety. In some embodiments, the antibody may have a sequence as shown by SEQ ID NO: 1, 2, 17, 18, 33, 34, 49, 50, 65, 66, 81, 82, 916, 25-32, 41-48, 57-64, 73-80, 8996 of WO2016094566.

In some embodiments, the antibodies (or fragments thereof) encoded by the viral genomes of the present disclosure may be any of those taught in U.S. Pat. Nos. 8,778,343 and 9,125,846, International Publications WO2012051498 and WO2011026031, or United States Publication Nos. US20150004169 and US20150322143, the contents of each of which are herein incorporated by reference in their entirety. Such antibodies may include those that bind to oligomeric species of tau. Further, such an antibody may be referred to as TOMA (tau oligomer monoclonal antibody), as described in Castillo-Carranza et at (Castillo-Carranza, D L et al., 2014 J Neurosci 34(12)4260-72) the contents of which are herein incorporated by reference in their entirety. In some embodiments, the antibody that binds oligomeric tau may be TTC-99.

In some embodiments, the antibodies (or fragments thereof) encoded by the viral genomes of the present disclosure may be any of those taught in international Publications WO2014059442, the contents of which are herein incorporated by reference in their entirety. Such antibodies may include those that bind to oligomeric species of tau.

In some embodiments, the antibodies (or fragments thereof) encoded by the viral genomes of the present disclosure may be any of those taught in the International Publications WO2014008404 and WO2016126993, United States Patent Publication 0520150183855, Yanamandra, K et al., 2013 Neuron 80(2):402-14 and Yanamandra, K et al 2015 Ann Clin Transl Neurol 2(3):278-88, the contents of each of which are herein incorporated by reference in their entirety. Such antibodies may block tau seeding. Non-limiting examples of antibodies described in these publications include HJ8.1.1, HJ8.1.2, HJ8.2, HJ8.3, HJ8.4, HJ8.5, HJ8.7, R18.8, HJ9.1, HJ9.2, HJ9,3, HJ9.4, HJ9.5, and variants thereof. Non-limiting examples of targeted epitopes of tau may include amino acids 2234, 385-391, 405-411, 3-6, 118-122, 386-401, 7-13, and/or 272-281 of human tau.

In some embodiments, the antibodies (or fragments thereof) encoded by the viral genomes of the present disclosure may be any of those taught in the international Publications WO2002062851, the contents of which are herein incorporated by reference in their entirety.

In some embodiments, the antibodies (or fragments thereof) encoded by the viral genomes of the present disclosure may be as described in Bright, J et al., 2015 Neurobiol of Aging 36:693-709; Pedersen, J T and Sigurdsson E M, 2015 Trends Mol Med 21(6):394-402; Levites, Y et al 2015 J Neurosci 35(16)6265-76; Jicha et al 1999 J Neurosci 19(17):7486-94; Reyes J F et al., 2012 Acta Neuropathol 123(1):119-32; Reynolds MIR, et al., 2006 J Neurosci. 26(42):10636-45; Gamblin, T C et al 2003 PNAS 100(17):10032-7; Castillo-Carranza, D L et al., 2014 J Neurosci 34(12)4260-72; Walls, K C et al., 2014 Neurosci Lett 575:96-100; Yanamandra, K. et al., 2013 Neuron 80(2):402-14; Yanamandra, K. et al 2015 Ann Clin Transl Neurol 2(3):278-88; Allen B, et al., 2002 J Neurosci 22(21):9340-51; Gotz, J et. al., 2010 Biochem Biophys Acta 1802(10):860-71; Hasegawa, M et al 1996 FEBS Lett 384:25-30; Carmel, G et. al. 1996 J Biol Chem 271(50:32780-32795; Jicha, G A et al. 1997 J Neurosci Res 48(2):128-132; Jicha, G A et al., 1997 J Neurochem 69(5):2087-95; the contents of each of which are herein incorporated by reference in their entirety.

Antibodies or fragments thereof encoded by the viral genomes of the present disclosure may be any commercially available anti-tau antibody known in the art or developed by a person with skill in the art. Non-limiting examples of commercially available anti-tau antibodies include EPR2396(2) (pThr⁵⁰; Abcam, Cambridge, MA), 5H911 (pThr¹⁸¹; ThermoFisher, Waltham, MA), M7004D06 (pThr¹⁸¹; BioLegend, San Diego, CA), 1E7 (pThr¹⁸¹; EMI) Millipore, Billerica, MA), EPR2400 (pSer¹⁹; Abcam, Cambridge, MA), EPR2401Y (pSer¹⁹⁹; Abcam, Cambridge, MA), 2H23L4 (pSer¹⁹⁹; ThermoFisher, Waltham, MA), EPR2402 (pSer²⁰²; Abcam, Cambridge, MA), 10F8 (pSer²⁰²; Abcam, Cambridge, MA), EPR2403(2) (pThr²⁰⁵; Abcam, Cambridge, MA), EPR1884(2) (pSer²¹⁴; Abcam, Cambridge, MA), EPR2488 (pThr²³¹; Abcam, Cambridge, MA), 1H6L6 (pThr²³¹; ThermoFisher, Waltham, MA), 3G3 (pThr²³¹, pSer²³⁵; Abcam, Cambridge, MA), EPR2452 (pSer²³⁵; Abcam, Cambridge, MA), 12610 (pSer²³⁸; Abcam, Cambridge, MA), EPR2454 (pSer²⁶²; Abcam, Cambridge, MA), EPR2457(2) (pSer³²⁴; Abcam, Cambridge, MA), EPR2603 (pSer³⁵⁶; Abcam, Cambridge, MA), EPR2731 (pSer³⁹⁶; Abcam, Cambridge, MA), EPR2605 (pSer⁴⁰⁴; Abcam, Cambridge, MA), EPR2866 (pSer⁴²²; Abcam, Cambridge, MA), 1A4 (pTau¹¹⁷; Origene, Rockville, MD), 7G9 (pTau¹⁷⁷⁻¹⁸⁷; Origene, Rockville, MD), 9B4 (pTau¹⁷⁷⁻¹⁸⁷; Origene, Rockville, MD), 2A4 (pTau¹⁷⁷⁻¹⁸⁷. Origene, Rockville, MD), 9G3 (pTyr¹⁸; NovusBio, Littleton, CO), EPR2455(2) (pSer⁶¹⁰; Abcam, Cambridge, MA), EP2456Y (pSer⁶²²; Abcam, Cambridge, MA; EMD Millipore, Billerica, MA), Slip 151 (PHF Tau⁹⁵⁻¹⁰⁸; BioLegend, San Diego, CA), TOMA-1 (Oligomeric Tau; ENID Millipore, Billerica, MA), Tau-nY18 (nTyr¹⁸; Origene, Rockville, MD; BioLegend, San Diego, CA; EMD Millipore, Billerica, MA), Tau-nY29 (nTyr²⁹; BioLegend, San Diego, CA; EMD Millipore, Billerica, MA; Abcam, Cambridge, MA), 1C9.G6 (di-methyl-Lys²⁸¹; BioLegend, San Diego, CA), 7G5.F4 (di-methyl-Lys³¹¹; BioLegend, San Diego, CA), TNT-1 (Taut-18; EMD Millipore, Billerica, MA), TNT-2 (Tau²⁻¹⁸; EMD Millipore, Billerica, MA), 7B8 (Tate-12; Abcam, Cambridge, MA), Tau-13 (Tau²⁰⁻³⁵; BioLegend, San Diego, CA), 1-100 (Tau¹⁻¹⁰⁰, BioLegend, San Diego, CA), 2G9.F10 (Tau¹⁵⁷⁻¹⁶⁸; BioLegend, San Diego, CA; Origene, Rockville, MD), 39E10 (Tau¹⁸⁹⁻¹⁹⁵; BioLegend, San Diego, CA; Origene, Rockville, MD), 77E9 (Tau 185-195; BioLegend, San Diego, CA; Origene; Rockville, MD), AT8 (pSer²⁰⁵, pSer²⁰⁵; ThermoFisher, Waltham, MA), A.717100 (pSer²¹², pSer²¹⁴; ThermoFisher, Waltham, MA), PHF-6 (pThr²³¹; NovusBio, Littleton, CO; EMD Millipore, Billerica, MA; BioLegend, San Diego, CA; ThermoFisher, Waltham; MA), AT180 (pThr²³¹; ThermoFisher, Waltham, MA), AT270 (pThr¹⁸¹; ThermoFisher, Waltham, MA), PHF-13 (pSer³⁹⁶; ThermoFisher, Waltham, MA; BioLegend, San Diego, CA), Tau³¹⁶⁻⁴²¹ BioLegend, San Diego, CA; EMD Millipore, Billerica, MA; ThermoFisher, Waltham, MA), Tau12 (Tau⁶⁻¹⁸; BioLegend, San Diego, CA; EMD Millipore, Billerica, MA), Tau5 (Tau²¹⁰⁻²⁴¹; BioLegend, San Diego, CA; EMD Millipore, Billerica, MA; Abcam, Cambridge MA; ThermoFisher, Waltham, MA), HT7 (Tau 159-163; ThermoFisher, Waltham, MA), 77G7 (Tau 316-355; BioLegend, San Diego, CA), Tau46 (Tau404-441; BioLegend, San Diego, CA; NovusBio, Littleton, CO; Abcam, Cambridge, MA), UMAB239; Origene, Rockville, MD), UMAB239 (Tau 623-758; Origene, Rockville, MD), OTI13B5 (Tau 623-758; Origene, Rockville, MD) (Tau⁶²³⁻⁷⁵⁸; Origene, Rockville, MD), OTI13B5 (Tau⁶²³⁻⁷⁵⁸; Origene, Rockville, MD), E178 (Tau⁷⁰⁰⁻⁸⁰⁰; Abcam, Cambridge, MA), SP70 (N-terminal Tau; Origene, Rockville, MD; NovusBio, Littleton, CO; ThermoFisher, Waltham, MA; Abcam, Cambridge, MA), C45 (N-terminal Tau; Origene, Rockville, MD), Tau7 (C-terminal Tau; EMD Millipore, Billerica, MA), S.125.0 (C-terminal Tau; ThermoFisher, Waltham, MA), 8E6/C11 (Three-repeat Tau²⁰⁹⁻²²⁴; EMD Millipore, Billerica, MA), 1E1/A6 (Four-repeat Tau²⁷⁵⁻²⁹¹; EMD Millipore, Billerica, MA), 7D12.1 (Four-repeat Tau²⁷⁵⁻²⁹¹; EMD Millipore, Billerica, MA), 5C7 (Four-repeat Tau²⁶⁷⁻²⁷⁸; BioLegend, San Diego, CA; Origene, Rockville, MD), 5F9 (Four-repeat Tau²⁷⁵⁻²⁹¹; BioLegend, San Diego, CA; Origene, Rockville, MD), 3H6.H7 (0N Tau³⁹⁻⁵⁰; BioLegend, San Diego, CA; Origene, Rockville, MD), 4H5.B9 (1N Tau⁶⁸⁻⁷⁹; BioLegend, San Diego, CA; Origene, Rockville, MD), 71C11 (2N Tau; BioLegend, San Diego, CA), PC1C6 (unphosphorylated tau; EMD Millipore, Billerica., MA), Tau2 (BioLegend, San Diego, CA; Origene, Rockville, MD; EMI) Millipore, Billerica, MA), 2E9 (Origene, Rockville, MD; NovusBio, Littleton, CO), 4F1 (Origene, Rockville, MD; NovusBio, Littleton, CO), 5B10 (NovusBio, Littleton, CO); 5E2 (EMD Millipore, Billerica, MA), Tau-93 (Origene, Rockville, MD), T14 (ThermoFisher, Waltham, MA), T46 (ThermoFisher, Waltham, MA), 13172 (ThermoFisher, Waltham, MA) and/or variants or derivates thereof.

In some embodiments, the antibodies encoded by the viral genomes of the present disclosure may be multispecific antibodies for transferrin receptor and a brain antigen, wherein the brain antigen may be tau, as described in International Publication WO2016081643, the contents of which are herein incorporated by reference in their entirety. In some embodiments, the antibody may have a sequence as given by SEQ ID NO: 160 or 161 of WO2016081643.

In some embodiments, the antibodies encoded by the viral genomes of the present disclosure are any of those described in U.S. Pat. Nos. 8,871,447, 8,420,613, International Publication No. WO2014193935, WO2010011999, or in United States Publication Nos. US20110250217, US20110020237, US20100316590, or US20120225864, the contents of each of which are herein incorporated by reference in their entirety. In some embodiments, the antibody recognizes an amyloidogenic or aggregating protein.

### Foodborne Illness and Gastroenteritis Related Antibodies

In some embodiments, the payload region of the viral particle comprises one or more nucleic acid sequences encoding one or more of the gastrointestinal and food illness related payload antibody polypeptides listed in Tables 14-20.

In some embodiments, the payload region of the viral particle comprises one or more nucleic acid sequences encoding one or more of the payload antibody polypeptides listed in Table 14 against Clostridium Difficile toxins (CD1-CD141; SEQ ID NO: 22301-22441).

TABLE 14
Antibodies against Clostridium Difficile toxins

| Antibody | | Antibody | | |
|---|---|---|---|---|
| No. | Description | Name | Reference Information | SEQ ID NO |
| CD1 | Camelid heavy chain only, Toxin | | WO2015100409 SEQ ID | 22301 |
| | A and B, | | NO: 164 | |
| CD2 | Camelid heavy chain only, Toxin | | WO2015100409 SEQ ID | 22302 |
| | A and B, | | NO: 165 | |
| CD3 | Camelid heavy chain only, Toxin | | WO2015100409 SEQ ID | 22303 |
| | A and B, | | NO: 166 | |
| CD4 | Camelid heavy chain only, Toxin | | WO2015100409 SEQ ID | 22304 |
| | A and B, | | NO: 167 | |
| CD5 | Heavy chain variable region, | PA-39 | U.S. Pat. No. 8,986,697 | 22305 |
| | toxin A | | SEQ ID NO: 1 | |
| CD6 | Heavy chain variable region, | PA-39 | U.S. Pat. No. 8,986,697 | 22306 |
| | toxin A | | SEQ ID NO: 2 | |
| CD7 | Heavy chain variable region, | PA-50 | U.S. Pat. No. 8,986,697 | 22307 |
| | toxin A | | SEQ ID NO: 5 | |
| CD8 | Heavy chain variable region, | PA-50 | U.S. Pat. No. 8,986,697 | 22308 |
| | toxin A | | SEQ ID NO: 6 | |
| CD9 | Heavy chain variable region, | | US20130202618 SEQ ID | 22309 |
| | toxin A | | NO: 1 | |
| CD10 | Heavy chain variable region, | | US20130202618 SEQ ID | 22310 |
| | toxin A | | NO: 2 | |
| CD11 | Heavy chain variable region, | | US20130202618 SEQ ID | 22311 |
| | toxin A | | NO: 5 | |
| CD12 | Heavy chain variable region, | | US20130202618 SEQ ID | 22312 |
| | toxin A | | NO: 6 | |
| CD13 | Heavy chain variable region, | H1H3067N | US20130230531 SEQ ID | 22313 |
| | toxin A and/or toxin B | | NO: 34 | |
| CD14 | Heavy chain variable region, | H1H3134N | US20130230531 SEQ ID | 22314 |
| | toxin A and/or toxin B | | NO: 18 | |
| CD15 | Heavy chain variable region, | H1H3117N | US20130230531 SEQ ID | 22315 |
| | toxin A and/or toxin B | | NO: 2 | |
| CD16 | Heavy chain variable region, | H1H3123N | US20130230531 SEQ ID | 22316 |
| | toxin A and/or toxin B | | NO: 66 | |
| CD17 | Heavy chain variable region, | H1H3121N | US20130230531 SEQ ID | 22317 |
| | toxin A and/or toxin B | | NO: 50 | |
| CD18 | Heavy chain variable region, | H1H3124N | US20130230531 SEQ ID | 22318 |
| | toxin A and/or toxin B | | NO: 82 | |
| CD19 | Heavy chain variable region, | H1H3328P | US20130230531 SEQ ID | 22319 |
| | toxin A and/or toxin B | | NO: 130 | |
| CD20 | Heavy chain variable region, | H1H3324P | US20130230531 SEQ ID | 22320 |
| | toxin A and/or toxin B | | NO: 98 | |
| CD21 | Heavy chain variable region, | H1H3325P | US20130230531 SEQ ID | 22321 |
| | toxin A and/or toxin B | | NO: 114 | |
| CD22 | Heavy chain variable region, | H1H3330P | US20130230531 SEQ ID | 22322 |
| | toxin A and/or toxin B | | NO: 146 | |
| CD23 | Heavy chain variable region, | H1H3350P | US20130230531 SEQ ID | 22323 |
| | toxin A and/or toxin B | | NO: 162 | |
| CD24 | Heavy chain variable region, | H1H3347P | US20130230531 SEQ ID | 22324 |
| | toxin A and/or toxin B | | NO: 274 | |
| CD25 | Heavy chain variable region, | H1H3335P | US20130230531 SEQ ID | 22325 |
| | toxin A and/or toxin B | | NO: 194 | |
| CD26 | Heavy chain variable region, | H1H3344P | US20130230531 SEQ ID | 22326 |
| | toxin A and/or toxin B | | NO: 258 | |
| CD27 | Heavy chain variable region, | H1H3339P | US20130230531 SEQ ID | 22327 |
| | toxin A and/or toxin B | | NO: 226 | |
| CD28 | Heavy chain variable region, | H1H3337P | US20130230531 SEQ ID | 22328 |
| | toxin A and/or toxin B | | NO: 210 | |
| CD29 | Heavy chain variable region, | H1H3343P | US20130230531 SEQ ID | 22329 |
| | toxin A and/or toxin B | | NO: 242 | |
| CD30 | Heavy chain variable region, | H1H3411P | US20130230531 SEQ ID | 22330 |
| | toxin A and/or toxin B | | NO: 354 | |
| CD31 | Heavy chain variable region, | H1H3354P | US20130230531 SEQ ID | 22331 |
| | toxin A and/or toxin B | | NO: 290 | |
| CD32 | Heavy chain variable region, | H1H3317P | US20130230531 SEQ ID | 22332 |
| | toxin A and/or toxin B | | NO: 178 | |
| CD33 | Heavy chain variable region, | H1H3355P | US20130230531 SEQ ID | 22333 |
| | toxin A and/or toxin B | | NO: 306 | |
| CD34 | Heavy chain variable region, | H1H3394P | US20130230531 SEQ ID | 22334 |
| | toxin A and/or toxin B | | NO: 322 | |
| CD35 | Heavy chain variable region, | H1H3401P | US20130230531 SEQ ID | 22335 |
| | toxin A and/or toxin B | | NO: 338 | |
| CD36 | Heavy chain variable region, | PA-41 | U.S. Pat. No. 8,986,697 | 22336 |
| | toxin B | | SEQ ID NO: 8 | |
| CD37 | Heavy chain variable region, | PA-41 | U.S. Pat. No. 8,986,697 | 22337 |
| | toxin B | | SEQ ID NO: 9 | |
| CD38 | Heavy chain variable region, | | US20130202618 SEQ ID | 22338 |
| | toxin B | | NO: 8 | |
| CD39 | Heavy chain variable region, | | US20130202618 SEQ ID | 22339 |
| | toxin B | | NO: 9 | |
| CD40 | Heavy chain, toxin A | 3D8 | U.S. Pat. No. 8,609,111 | 22340 |
| | | | SEQ ID NO: 1 | |
| CD41 | Heavy chain, toxin A | 1B11 | U.S. Pat. No. 8,609,111 | 22341 |
| | | | SEQ ID NO: 2 | |
| CD42 | Heavy chain, toxin A | 33.3H2 | U.S. Pat. No. 8,609,111 | 22342 |
| | | | SEQ ID NO: 3 | |
| CD43 | Heavy chain, toxin A | | US20140004118 SEQ ID | 22343 |
| | | | NO: 89 | |
| CD44 | Heavy chain, toxin A | | US20140004118 SEQ ID | 22344 |
| | | | NO: 93 | |
| CD45 | Heavy chain, toxin B | | US20130058962 SEQ ID | 22345 |
| | | | NO: 65 | |
| CD46 | Heavy chain, toxin B | Bezlotoxumab | | 22346 |
| CD47 | Heavy-chain-only, toxin A | | US20130058962 SEQ ID | 22347 |
| | | | NO: 59 | |
| CD48 | Heavy-chain-only, toxin A | | US20130058962 SEQ ID | 22348 |
| | | | NO: 60 | |
| CD49 | Heavy-chain-only, toxin A | | US20130058962 SEQ ID | 22349 |
| | | | NO: 61 | |
| CD50 | Heavy-chain-only, toxin A | | US20130058962 SEQ ID | 22350 |
| | | | NO: 62 | |
| CD51 | Heavy-chain-only, toxin A | | US20130058962 SEQ ID | 22351 |
| | | | NO: 63 | |
| CD52 | Heavy-chain-only, toxin A | | US20130058962 SEQ ID | 22352 |
| | | | NO: 64 | |
| CD53 | Heavy-chain-only, toxin A | | US20130058962 SEQ ID | 22353 |
| | | | NO: 87 | |
| CD54 | Heavy-chain-only, toxin A | | US20130058962 SEQ ID | 22354 |
| | | | NO: 95 | |
| CD55 | Heavy-chain-only, toxin B | 124-152 | U.S. Pat. No. | 22355 |
| | | | 8,609,111 | |
| | | | SEQ ID NO: 54 | |
| CD56 | Heavy-chain-only, toxin B | | US20130058962 SEQ ID | 22356 |
| | | | NO: 66 | |
| CD57 | Heavy-chain-only, toxin B | | US20130058962 SEQ ID | 22357 |
| | | | NO: 67 | |
| CD58 | Heavy-chain-only, toxin B | | US20130058962 SEQ ID | 22358 |
| | | | NO: 68 | |
| CD59 | Heavy-chain-only, toxin B | | US20130058962 SEQ ID | 22359 |
| | | | NO: 69 | |
| CD60 | Heavy-chain-only, toxin B | | US20130058962 SEQ ID | 22360 |
| | | | NO: 70 | |
| CD61 | Heavy-chain-only, toxin B | | US20130058962 SEQ ID | 22361 |
| | | | NO: 71 | |
| CD62 | Heavy-chain-only, toxin B | | US20130058962 SEQ ID | 22362 |
| | | | NO: 72 | |
| CD63 | Heavy-chain-only, toxin B | | US20130058962 SEQ ID | 22363 |
| | | | NO: 73 | |
| CD64 | Heavy-chain-only, toxin B | | US20130058962 SEQ ID | 22364 |
| | | | NO: 74 | |
| CD65 | Heavy-chain-only, toxin B | | US20130058962 SEQ ID | 22365 |
| | | | NO: 75 | |
| CD66 | Heavy-chain-only, toxin B | | US20130058962 SEQ ID | 22366 |
| | | | NO: 76; SEQ ID NO: 87; | |
| | | | SEQ ID NO: 95 | |
| CD67 | Light chain variable region, toxin | PA-39 | U.S. Pat. No. 8,986,697 | 22367 |
| | A | | SEQ ID NO: 3 | |
| CD68 | Light chain variable region, toxin | PA-39 | U.S. Pat. No. 8,986,697 | 22368 |
| | A | | SEQ ID NO: 4 | |
| CD69 | Light chain variable region, toxin | PA-50 | U.S. Pat. No. 8,986,697 | 22369 |
| | A | | SEQ ID NO: 7 | |
| CD70 | Light chain variable region, toxin | | US20130202618 SEQ ID | 22370 |
| | A | | NO: 3 | |
| CD71 | Light chain variable region, toxin | | US20130202618 SEQ ID | 22371 |
| | A | | NO: 4 | |
| CD72 | Light chain variable region, toxin | | US20130202618 SEQ ID | 22372 |
| | A | | NO: 7 | |
| CD73 | Light chain variable region, toxin | H1H3067N | US20130230531 SEQ ID | 22373 |
| | A and/or toxin B | | NO: 42 | |
| CD74 | Light chain variable region, toxin | H1H3134N | US20130230531 SEQ ID | 22374 |
| | A and/or toxin B | | NO: 26 | |
| CD75 | Light chain variable region, toxin | H1H3117N | US20130230531 SEQ ID | 22375 |
| | A and/or toxin B | | NO: 10 | |
| CD76 | Light chain variable region, toxin | H1H3123N | US20130230531 SEQ ID | 22376 |
| | A and/or toxin B | | NO: 74 | |
| CD77 | Light chain variable region, toxin | H1H3121N | US20130230531 SEQ ID | 22377 |
| | A and/or toxin B | | NO: 58 | |
| CD78 | Light chain variable region, toxin | H1H3124N | US20130230531 SEQ ID | 22378 |
| | A and/or toxin B | | NO: 90 | |
| CD79 | Light chain variable region, toxin | H1H3328P | US20130230531 SEQ ID | 22379 |
| | A and/or toxin B | | NO: 138 | |
| CD80 | Light chain variable region, toxin | H1H3324P | US20130230531 SEQ ID | 22380 |
| | A and/or toxin B | | NO: 106 | |
| CD81 | Light chain variable region, toxin | H1H3325P | US20130230531 SEQ ID | 22381 |
| | A and/or toxin B | | NO: 122 | |
| CD82 | Light chain variable region, toxin | H1H3330P | US20130230531 SEQ ID | 22382 |
| | A and/or toxin B | | NO: 154 | |
| CD83 | Light chain variable region, toxin | H1H3350P | US20130230531 SEQ ID | 22383 |
| | A and/or toxin B | | NO: 170 | |
| CD84 | Light chain variable region, toxin | H1H3347P | US20130230531 SEQ ID | 22384 |
| | A and/or toxin B | | NO: 282 | |
| CD85 | Light chain variable region, toxin | H1H3335P | US20130230531 SEQ ID | 22385 |
| | A and/or toxin B | | NO: 202 | |
| CD86 | Light chain variable region, toxin | H1H3344P | US20130230531 SEQ ID | 22386 |
| | A and/or toxin B | | NO: 266 | |
| CD87 | Light chain variable region, toxin | H1H3339P | US20130230531 SEQ ID | 22387 |
| | A and/or toxin B | | NO: 234 | |
| CD88 | Light chain variable region, toxin | H1H3337P | US20130230531 SEQ ID | 22388 |
| | A and/or toxin B | | NO: 218 | |
| CD89 | Light chain variable region, toxin | H1H3343P | US20130230531 SEQ ID | 22389 |
| | A and/or toxin B | | NO: 250 | |
| CD90 | Light chain variable region, toxin | H1H3411P | US20130230531 SEQ ID | 22390 |
| | A and/or toxin B | | NO: 362 | |
| CD91 | Light chain variable region, toxin | H1H3354P | US20130230531 SEQ ID | 22391 |
| | A and/or toxin B | | NO: 298 | |
| CD92 | Light chain variable region, toxin | H1H3317P | US20130230531 SEQ ID | 22392 |
| | A and/or toxin B | | NO: 186 | |
| CD93 | Light chain variable region, toxin | H1H3355P | US20130230531 SEQ ID | 22393 |
| | A and/or toxin B | | NO: 314 | |
| CD94 | Light chain variable region, toxin | H1H3394P | US20130230531 SEQ ID | 22394 |
| | A and/or toxin B | | NO: 330 | |
| CD95 | Light chain variable region, toxin | H1H3401P | US20130230531 SEQ ID | 22395 |
| | A and/or toxin B | | NO: 346 | |
| CD96 | Light chain variable region, toxin | PA-41 | U.S. Pat. No. 8,986,697 | 22396 |
| | B | | SEQ ID NO: 10 | |
| CD97 | Light chain variable region, toxin | | US20130202618 SEQ ID | 22397 |
| | B | | NO: 10 | |
| CD98 | Light chain, toxin A | 3D8 | U.S. Pat. No. 8,609,111 | 22398 |
| | | | SEQ ID NO: 4 | |
| CD99 | Light chain, toxin A | 1B11 | U.S. Pat. No. 8,609,111 | 22399 |
| | | | SEQ ID NO: 5 | |
| CD100 | Light chain, toxin A | 33.3H2 | U.S. Pat. No. 8,609,111 | 22400 |
| | | | SEQ ID NO: 6 | |
| CD101 | Light chain, toxin A | | US20140004118 SEQ ID | 22401 |
| | | | NO: 91 | |
| CD102 | Light chain, toxin A | | US20140004118 SEQ ID | 22402 |
| | | | NO: 95 | |
| CD103 | Light chain, toxin B | 124-152 | U.S. Pat. No. 8,609,111 | 22403 |
| | | | SEQ ID NO: 58 | |
| CD104 | Light chain, toxin B | Bezlotoxumab | | 22404 |
| CD105 | Recombinant camelid heavy | | WO2015100409 SEQ ID | 22405 |
| | chain only, Toxin A and B | | NO: 87 | |
| CD106 | Recombinant camelid heavy | | WO2015100409 SEQ ID | 22406 |
| | chain only, Toxin A and B | | NO: 95 | |
| CD107 | Recombinant camelid heavy- | | WO2015100409 SEQ ID | 22407 |
| | chain-only, toxin A | | NO: 59 | |
| CD108 | Recombinant camelid heavy- | | WO2015100409 SEQ ID | 22408 |
| | chain-only, toxin A | | NO: 60 | |
| CD109 | Recombinant camelid heavy - | | WO2015100409 SEQ ID | 22409 |
| | chain-only, toxin A | | NO: 61 | |
| CD110 | Recombinant camelid heavy- | | WO2015100409 SEQ ID | 22410 |
| | chain-only, toxin A | | NO: 62 | |
| CD111 | Recombinant camelid heavy- | | WO2015100409 SEQ ID | 22411 |
| | chain-only, toxin A | | NO: 63 | |
| CD112 | Recombinant camelid heavy- | | WO2015100409 SEQ ID | 22412 |
| | chain-only, toxin A | | NO: 64 | |
| CD113 | Recombinant camelid heavy- | | WO2015100409 SEQ ID | 22413 |
| | chain-only, toxin B | | NO: 65 | |
| CD114 | Recombinant camelid heavy- | | WO2015100409 SEQ ID | 22414 |
| | chain-only, toxin B | | NO: 66 | |
| CD115 | Recombinant camelid heavy- | | WO2015100409 SEQ ID | 22415 |
| | chain-only, toxin B | | NO: 67 | |
| CD116 | Recombinant camelid heavy- | | WO2015100409 SEQ ID | 22416 |
| | chain-only, toxin B | | NO: 68 | |
| CD117 | Recombinant camelid heavy- | | WO2015100409 SEQ ID | 22417 |
| | chain-only, toxin B | | NO: 69 | |
| CD118 | Recombinant camelid heavy - | | WO2015100409 SEQ ID | 22418 |
| | chain-only, toxin B | | NO: 70 | |
| CD119 | Recombinant camelid heavy- | | WO2015100409 SEQ ID | 22419 |
| | chain-only, toxin B | | NO: 71 | |
| CD120 | Recombinant camelid heavy- | | WO2015100409 SEQ ID | 22420 |
| | chain-only, toxin B | | NO: 72 | |
| CD121 | Recombinant camelid heavy- | | WO2015100409 SEQ ID | 22421 |
| | chain-only, toxin B | | NO: 73 | |
| CD122 | Recombinant camelid heavy- | | WO2015100409 SEQ ID | 22422 |
| | chain-only, toxin B | | NO: 74 | |
| CD123 | Recombinant camelid heavy- | | WO2015100409 SEQ ID | 22423 |
| | chain-only, toxin B | | NO: 75 | |
| CD124 | Recombinant camelid heavy- | | WO2015100409 SEQ ID | 22424 |
| | chain-only, toxin B | | NO: 76 | |
| CD125 | Toxin A | Actoxumab | | 22425 |
| CD126 | Toxin A | Actoxumab | | 22426 |
| CD127 | Toxin A | MK3415A | U.S. Pat. No. 7,625,559 | 22427 |
| | | (Actoxumab + | SEQ ID NO: 1 | |
| | | bezlo- | | |
| | | toxumab) | | |
| CD128 | Toxin A | MK3415A | U.S. Pat. No. 7,625,559 | 22428 |
| | | (Actoxumab + | SEQ ID NO: 4 | |
| | | bezlo- | | |
| | | toxumab) | | |
| CD129 | Toxin A | MK3415A | U.S. Pat. No. 7,625,559 | 22429 |
| | | (Actoxumab + | SEQ ID NO: 54 | |
| | | bezlo- | | |
| | | toxumab) | | |
| CD130 | Toxin A | MK3415A | U.S. Pat. No. 7,625,559 | 22430 |
| | | (Actoxumab + | SEQ ID NO: 58 | |
| | | bezlo | | |
| | | toxumab) | | |
| CD131 | Toxin A | A4.2 | US20130230537 SEQ ID | 22431 |
| | | | NO: 34 | |
| CD132 | Toxin A | A5.1 | US20130230537 SEQ ID | 22432 |
| | | | NO: 35 | |
| CD133 | Toxin A | A19.2 | US20130230537 SEQ ID | 22433 |
| | | | NO: 36 | |
| CD134 | Toxin A | A20.1 | US20130230537 SEQ ID | 22434 |
| | | | NO: 37 | |
| CD135 | Toxin A | A24.1 | US20130230537 SEQ ID | 22435 |
| | | | NO: 38 | |
| CD136 | Toxin A | A26.8 | US20130230537 SEQ ID | 22436 |
| | | | NO: 39 | |
| CD137 | Toxin B | B5.2 | US20130230537 SEQ ID | 22437 |
| | | | NO: 40 | |
| CD138 | Toxin B | B7.3 | US20130230537 SEQ ID | 22438 |
| | | | NO: 41 | |
| CD139 | Toxin B | B13.6 | US20130230537 SEQ ID | 22439 |
| | | | NO: 42 | |
| CD140 | Toxin B | B15.3 | US20130230537 SEQ ID | 22440 |
| | | | NO: 43 | |
| CD141 | Toxin B | B15.5 | US20130230537 SEQ ID | 22441 |
| | | | NO: 44 | |

In some embodiments, the payload region of the viral particle comprises one or more nucleic acid sequences encoding one or more of the payload antibody polypeptides listed in Table 15 against Campylobacter jejuni (CAMP 1-CAMP10, SEQ ID NO: 22442-22451).

TABLE 15
Antibodies against Campylobacter jejuni

| Antibody | | | | |
|---|---|---|---|---|
| No. | Description | Antibody Name | Reference Information | SEQ ID NO |
| CAMP1 | Consensus | FlagV1 | WO2014063253 SEQ ID NO: 7 | 22442 |
| CAMP2 | — | FlagV1M | WO2014063253 SEQ ID NO: 8 | 22443 |
| CAMP3 | — | FlagV1F23M | WO2014063253 SEQ ID NO: 9 | 22444 |
| CAMP4 | — | Flag V1MDSB | WO2014063253 SEQ ID NO: 10 | 22445 |
| CAMP5 | — | FlagV1MDSB | WO2014063253 SEQ ID NO: 11 | 22446 |
| CAMP6 | Consensus | FlagV6 | WO2014063253 SEQ ID NO: 12 | 22447 |
| CAMP7 | — | FlagV6M | WO2014063253 SEQ ID NO: 13 | 22448 |
| CAMP8 | — | FlagV6F23M | WO2014063253 SEQ ID NO: 14 | 22449 |
| CAMP9 | — | FlagV6MDSB | WO2014063253 SEQ ID NO: 15 | 22450 |
| CAMP10 | — | FlagV6F23MDSB | WO2014063253 SEQ ID NO: 16 | 22451 |

In some embodiments, the payload region of the viral particle comprises one or more nucleic acid sequences encoding one or more of the payload antibody polypeptides listed in Table 16 against bacterial infections of the intestine (BACG1-BACG98; SEQ ID NO: 22452-22549).

TABLE 16
Antibodies against bacterial infections of the intestine

| Antibody | | Antibody | Reference | SEQ |
|---|---|---|---|---|
| No. | Description | Name | Information | ID NO |
| BACG1 | Antibody against Listeria monocytogenes | Antibody | CN103497252 | 22452 |
| | | from | SEQ ID NO: 1 | |
| | | CN10349 | | |
| | | 7252 | | |
| BACG2 | Bivalent monovalent antibody against Pseudomonas, | anti- | U.S. Pat. No. | 22453 |
| | Clostridium, Staphylococcus, Pasteurella, Yersinia, | LYS3- | 7,655,759 | |
| | Bacillus anthracis, Neisseria, Vibrio, enterotoxic E. coli. | long | SEQ ID NO: | |
| | Salmonella, Shigella, and Listeria, Clostridium, | hinge/ | 22 | |
| | Staphylococcus, Pseudomonas, Pasteurella, Yersinia, | Cys-Tag. | | |
| | Bacillus anthracis, Neisseria, Vibrio, enterotoxic E. coli., | | | |
| | Salmonella, Shigella, and Listeria bacteria | | | |
| BACG3 | Heavy chain only, Antibody against Pseudomonas, | LYS2 | U.S. Pat. No. | 22454 |
| | Clostridium, Staphylococcus, Pasteurella, Yersinia, | VHH | 7,655,759 | |
| | Bacillus anthracis, Neisseria, Vibrio, enterotoxic E. coli., | | SEQ ID NO: | |
| | Salmonella, Shigella, and Listeria, Clostridium, | | 18 | |
| | Staphylococcus, Pseudomonas, Pasteurella, Yersinia, | | | |
| | Bacillus anthracis, Neisseria, Vibrio, enterotoxic E. coli., | | | |
| | Salmonella, Shigella, and Listeria bacteria | | | |
| BACG4 | Heavy chain only, Antibody against Pseudomonas, | LYS3 | U.S. Pat. No. | 22455 |
| | Clostridium, Staphylococcus, Pasteurella, Yersinia, | VHH | 7,655,759 | |
| | Bacillus anthracis, Neisseria, Vibrio, enterotoxic E. coli. | | SEQ ID NO: | |
| | Salmonella, Shigella, and Listeria, Clostridium, | | 24 | |
| | Staphylococcus, Pseudomonas, Pasteurella, Yersinia, | | | |
| | Bacillus anthracis, Neisseria, Vibrio, enterotoxic E. coli., | | | |
| | Salmonella, Shigella, and Listeria bacteria | | | |
| BACG5 | Heavy chain segment including variable region, | F10 | U.S. Pat. No. | 22456 |
| | Starhylococcus enterotoxin B | | 8,895,704 | |
| | | | SEQ ID NO: | |
| | | | 30 | |
| BACG6 | Heavy chain variable region, Antibody against, P. | mAb 741 | U.S. Pat. No. | 22457 |
| | aeruginosa, Proteus Vulgaris, non-pathogenic E. coli., | | 8,263,078 | |
| | Citrobacter freundii, Serratia marcenscens, Enterobacter | | SEQ ID NO: 1 | |
| | cloacae, Campylobacter jejuni, Helicobacter pylori, | | | |
| | Salmonella typhimurium, Salmonella muenchen, Proteus | | | |
| | mirabilis and Enteropathogenic E. coli. | | | |
| BACG7 | Heavy chain variable region, Antibody against, P. | mAb 763 | U.S. Pat. No. | 22458 |
| | aeruginosa, Proteus Vulgaris, non-pathogenic E. coli., | | 8,263,078 | |
| | Citrobacter freundii, Serratia marcenscens, Enterobacter | | SEQ ID NO: 2 | |
| | cloacae, Campylobacter jejuni, Helicobacter pylori, | | | |
| | Salmonella typhimurium, Salmonella muenchen, Proteus | | | |
| | mirabilis and Enteropathogenic E. coli.., | | | |
| BACG8 | Heavy chain variable region, antibody against flagellin | | U.S. Pat. No. | 22459 |
| | from Salmonella or Pseudomonas | | 8,173,130 | |
| | | | SEQ ID NO: 1 | |
| BACG9 | Heavy chain variable region, Antibody against Gram | INO 743 | US20100239583 | 22460 |
| | negative (E. coli., Salmonella, Serratia, Proteus, | | SEQ ID | |
| | Enterobacter, Citrobacter, Campylobacter | | NO: 1 | |
| | and Pseudomonas) | | | |
| BACG10 | Heavy chain variable region, Antibody against | Abba3 | U.S. Pat. No. | 22461 |
| | Helicobacter pyroli | | 8,025,880 | |
| | | | SEQ ID NO: | |
| | | | 18 | |
| BACG11 | Heavy chain variable region, Antibody against | IgHV3- | U.S. Pat. No. | 22462 |
| | Helicobacter pyroli | 48*3 | 8,025,880 | |
| | | | SEQ ID NO: | |
| | | | 20 | |
| BACG12 | Heavy chain variable region, Antibody against | clone 5 | U.S. Pat. No. | 22463 |
| | Helicobacter pyroli | | 8,025,880 | |
| | | | SEQ ID NO: | |
| | | | 21 | |
| BACG13 | Heavy chain variable region, Antibody against | C4 | U.S. Pat. No. | 22464 |
| | Helicobacter pyroli | | 8,025,880 | |
| | | | SEQ ID NO: | |
| | | | 22 | |
| BACG14 | Heavy chain variable region, Antibody against | IgHV1- | U.S. Pat. No. | 22465 |
| | Helicobacter pyroli | 18*01 | 8,025,880 | |
| | | | SEQ ID NO: | |
| | | | 23 | |
| BACG15 | Heavy chain variable region, Antibody against | C5 | U.S. Pat. No. | 22466 |
| | Helicobacter pyroli | | 8,025,880 | |
| | | | SEQ ID NO: | |
| | | | 24 | |
| BACG16 | Heavy chain variable region, antibody against many | SWLA3 | WO20030079 | 22467 |
| | pathogens, | | 89 SEQ ID | |
| | | | NO: 4 | |
| BACG17 | Heavy chain variable region, antibody against | SWLA3 | US20040052814 | 22468 |
| | Streptococcus mutans, Escherichia coli, Shigella | | SEQ ID | |
| | dysenteriae, Salmonella typhimurium, Streptococcus | | NO: 4 | |
| | pneumoniae, Staphylococcus aureus, and Pseudomonas | | | |
| | aeruginosa | | | |
| BACG18 | Heavy chain variable region, antibody against | SWLA3 | US20040052814 | 22469 |
| | Staphylococcus mutans, Escherichia coli, Shigella | | SEQ ID | |
| | dysenteriae, Salmonella typhimurium, Streptococcus | | NO: 8 | |
| | pneumoniae, Staphylococcus aureus, and Pseudomonas | | | |
| | aeruginosa | | | |
| BACG19 | Heavy chain, antibody against E coli, Shigella, | Ab1 | WO2012162253 | 22470 |
| | Entaamoeba histolytica, Salmonella, Campylobacter, or | | SEQ ID | |
| | Clostridium difficile, rotavirus, RSV, HIV, norvovirus, | | NO: 4 | |
| | adenovirus, and astrovirus, other diseases causing | | | |
| | diarrhea, | | | |
| BACG20 | Heavy chain, antibody against E coli, Shigella, | Ab2 | WO2012162253 | 22471 |
| | Entaamoeba histolytica, Salmonella, Campylobacter, or | | SEQ ID | |
| | Clostridium difficile, rotavirus, RSV, HIV, norvovirus, | | NO: 14 | |
| | adenovirus, and astrovirus, other diseases causing | | | |
| | diarrhea, | | | |
| BACG21 | Heavy chain, antibody against E coli, Shigella, | Ab3 | WO2012162253 | 22472 |
| | Entaamoeba histolytica, Salmonella, Campylobacter, or | | SEQ ID | |
| | Clostridium difficile, rotavirus, RSV, HIV, norvovirus, | | NO: 24 | |
| | adenovirus, and astrovirus, other diseases causing | | | |
| | diarrhea, | | | |
| BACG22 | Heavy chain, antibody against E coli, Shigella, | Ab4 | WO2012162253 | 22473 |
| | Entaamoeba histolytica, Salmonella, Campylobacter, or | | SEQ ID | |
| | Clostridium difficile, rotavirus, RSV, HIV, norvovirus, | | NO: 34 | |
| | adenovirus, and astrovirus, other diseases causing | | | |
| | diarrhea, | | | |
| BACG23 | Heavy chain, antibody against E coli, Shigella, | Ab5 | WO2012162253 | 22474 |
| | Entaamoeba histolytica, Salmonella, Campylobacter, or | | SEQ ID | |
| | Clostridium difficile, rotavirus, RSV, HIV, norvovirus, | | NO: 44 | |
| | adenovirus, and astrovirus, other diseases causing | | | |
| | diarrhea, | | | |
| BACG24 | Heavy chain, antibody against E coli, Shigella, | Ab6 | WO2012162253 | 22475 |
| | Entaamoeba histolytica, Salmonella, Campylobacter, or | | SEQ ID | |
| | Clostridium difficile, rotavirus, RSV, HIV, norvovirus, | | NO: 54 | |
| | adenovirus, and astrovirus, other diseases causing | | | |
| | diarrhea | | | |
| BACG25 | Heavy chain, antibody against E coli, Shigella, | Ab7 | WO2012162253 | 22476 |
| | Entaamoeba histolytica, Salmonella, Campylobacter, or | | SEQ ID | |
| | Clostridium difficile, rotavirus, RSV, HIV, norvovirus, | | NO: 64 | |
| | adenovirus, and astrovirus, other diseases causing | | | |
| | diarrhea, | | | |
| BACG26 | Heavy chain, antibody against E coli, Shigella, | Ab8 | WO2012162253 | 22477 |
| | Entaamoeba histolytica, Salmonella, Campylobacter, or | | SEQ ID | |
| | Clostridium difficile, rotavirus, RSV, HIV, norvovirus, | | NO: 74 | |
| | adenovirus, and astrovirus, other diseases causing | | | |
| | diarrhea, | | | |
| BACG27 | Heavy chain, antibody against E coli, Shigella, | Ab9 | WO2012162253 | 22478 |
| | Entaamoeba histolytica, Salmonella, Campylobacter, or | | SEQ ID | |
| | Clostridium difficile, rotavirus, RSV, HIV, norvovirus, | | NO: 84 | |
| | adenovirus, and astrovirus, other diseases causing | | | |
| | diarrhea, | | | |
| BACG28 | Heavy chain, antibody against E coli, Shigella, | Ab10 | WO2012162253 | 22479 |
| | Entaamoeba histolytica, Salmonella, Campylobacter, or | | SEQ ID | |
| | Clostridium difficile, rotavirus, RSV, HIV, norvovirus, | | NO: 94 | |
| | adenovirus, and astrovirus, other diseases causing | | | |
| | diarrhea, | | | |
| BACG29 | Heavy chain, antibody against E coli, Shigella, | Ab11 | WO2012162253 | 22480 |
| | Entaamoeba histolytica, Salmonella, Campylobacter, or | | SEQ ID | |
| | Clostridium difficile, rotavirus, RSV, HIV, norvovirus, | | NO: 104 | |
| | adenovirus, and astrovirus, other diseases causing | | | |
| | diarrhea, | | | |
| BACG30 | Heavy chain, antibody against E coli, Shigella, | Ab12 | WO2012162253 | 22481 |
| | Entaamoeba histolytica, Salmonella, Campylobacter, or | | SEQ ID | |
| | Clostridium difficile, rotavirus, RSV, HIV, norvovirus, | | NO: 114 | |
| | adenovirus, and astrovirus, other diseases causing | | | |
| | diarrhea, | | | |
| BACG31 | Heavy chain, antibody against E coli, Shigella, | Ab13 | WO2012162253 | 22482 |
| | Entaamoeba histolytica, Salmonella, Campylobacter, or | | SEQ ID | |
| | Clostridium difficile, rotavirus, RSV, HIV, norvovirus, | | NO: 124 | |
| | adenovirus, and astrovirus, other diseases causing | | | |
| | diarrhea, | | | |
| BACG32 | Heavy chain, antibody against E coli, Shigella, | Ab14 | WO2012162253 | 22483 |
| | Entaamoeba histolytica, Salmonella, Campylobacter, or | | SEQ ID | |
| | Clostridium difficile, rotavirus, RSV, HIV, norvovirus, | | NO: 134 | |
| | adenovirus, and astrovirus, other diseases causing | | | |
| | diarrhea, | | | |
| BACG33 | Heavy chain, Antibody against Escherichia coli | | WO2014070117 | 22484 |
| | infection, Staphylococcus infection | | SEQ ID | |
| | | | NO: 3 | |
| BACG34 | Heavy chain, Antibody against Listeria | 6H8 | U.S. Pat. No. | 22485 |
| | monocytogenes or WR-tubercle bacillus | | 8,445,643 | |
| | | | SEQ ID NO: 5 | |
| BACG35 | Heavy chain, Antibody against Pseudomonas, | | U.S. Pat. No. | 22486 |
| | Clostridium, Staphylococcus, Pasteurella, Yersinia, | | 7,655,759 | |
| | Bacillus anthracis, Neisseria, Vibrio, enterotoxic E. coli, | | SEQ ID NO: | |
| | Salmonella, Shigella, and Listeria, Clostridium, | | 25 | |
| | Staphylococcus, Pseudomonas, Pasteurella, Yersinia, | | | |
| | Bacillus anthracis, Neisseria, Vibrio, enterotoxic E. coli, | | | |
| | Salmonella, Shigella, and Listeria bacteria | | | |
| BACG36 | Heavy chain, Antibody against Pseudomonas, | | U.S. Pat. No. | 22487 |
| | Clostridium, Staphylococcus, Pasteurella, Yersinia, | | 7,655,759 | |
| | Bacillus anthracis, Neisseria, Vibrio, enterotoxic E. coli, | | SEQ ID NO: | |
| | Salmonella, Shigella, and Listeria, Clostridium, | | 26 | |
| | Staphylococcus, Pseudomonas, Pasteurella, Yersinia, | | | |
| | Bacillus anthracis, Neisseria, Vibrio, enterotoxic E. coli, | | | |
| | Salmonella, Shigella, and Listeria bacteria | | | |
| BACG37 | Heavy chain, Starhylococcus enterotoxin B | 100C9 | U.S. Pat. No. | 22488 |
| | | | 8,895,704 | |
| | | | SEQ ID NO: | |
| | | | 34 | |
| BACG38 | Heavy chain, Starhylococcus enterotoxin B | 79G9+ | U.S. Pat. No. | 22489 |
| | | | 8,895,704 | |
| | | | SEQ ID NO: 38 | |
| BACG39 | Heavy chain, Starhylococcus enterotoxin B | 79G9 | U.S. Pat. No. | 22490 |
| | | | 8,895,704 | |
| | | | SEQ ID NO: 126 | |
| BACG40 | Heavy chain, Starhylococcus enterotoxin B | 154G12 | U.S. Pat. No. | 22491 |
| | | | 8,895,704 | |
| | | | SEQ ID NO: 142 | |
| BACG41 | Light chain variable region, Antibody against, P. | mAb 741 | U.S. Pat. No. | 22492 |
| | aeruginosa, Proteus Vulgaris, non-pathogenic E. Coli, | | 8,263,078 | |
| | Citrobacter freundii, Serratia marcenscens, Enterobacter | | SEQ ID NO: 3 | |
| | cloacae, Campylobacter jejuni, Helicobacter pylori, | | | |
| | Salmonella typhimurium, Salmonella muenchen, Proteus | | | |
| | mirabilis and Enteropathogenic E. Coli. | | | |
| BACG42 | Light chain variable region, Antibody against, P. | mAb 763 | U.S. Pat. No. | 22493 |
| | aeruginosa, Proteus Vulgaris, non-pathogenic E. Coli, | | 8,263,078 | |
| | Citrobacter freundii, Serratia marcenscens, Enterobacter | | SEQ ID NO: 4 | |
| | cloacae, Campylobacter jejuni, Helicobacter pylori, | | | |
| | Salmonella typhimurium, Salmonella muenchen, Proteus | | | |
| | mirabilis and Enteropathogenic E. Coli | | | |
| BACG43 | Light chain variable region, Antibody against E coli, | Ab1 | WO2012162253 | 22494 |
| | Shigella, Entaamoeba histolytica, Salmonella, | | SEQ ID | |
| | Campylobacter, or Clostridium difficile, rotavirus, RSV, | | NO: 1 | |
| | HIV, norvovirus, adenovirus, and astrovirus, other | | | |
| | diseases causing diarrhea | | | |
| BACG44 | Light chain variable region, antibody against flagellin | | U.S. Pat. No. | 22495 |
| | from Salmonella or Pseudomonas | | 8,173,130 | |
| | | | SEQ ID NO: 3 | |
| BACG45 | Light chain variable region, Antibody against Gram | INO 743 | US20100239583 | 22496 |
| | negative (E. coli, Salmonella, Serratia, Proteus, | | SEQ ID | |
| | Enterobacter, Citrobacter, Campylobacter | | NO: 2 | |
| | and Pseudomonas) | | | |
| BACG46 | Light chain variable region, Antibody against | Abba3 | U.S. Pat. No. | 22497 |
| | Helicobacter pyroli | | 8,025,880 | |
| | | | SEQ ID NO: 19 | |
| BACG47 | Light chain variable region, Antibody against many | SWLA3 | WO2003007989 | 22498 |
| | pathogens | | SEQ ID | |
| | | | NO: 7 | |
| BACG48 | Light chain, Antibody against E. coli, Shigaella, | Ab 1 | US201200294822 | 22499 |
| | Entaamoeba histolvtica, Salmonella, Campylobacter, | | SEQ ID | |
| | or Clostridium difficile or a virus selected from rotavirus, | | NO: 2 | |
| | RSV, HIV, norvovirus, adenovirus, and astrovirus | | | |
| BACG49 | Light chain, Antibody against E. coli, Shigaella, | Ab 1 | US201200294822 | 22500 |
| | Entaamoeba histolvtica, Salmonella, Campylobacter, | | SEQ ID | |
| | or Clostridium difficile or a virus selected from rotavirus, | | NO: 4 | |
| | RSV, HIV, norvovirus, adenovirus, and astrovirus | | | |
| BACG50 | Light chain, Antibody against E. coli, Shigaella, | Ab 2 | US201200294 | 22501 |
| | Entaamoeba histolvtica, Salmonella, Campylobacter, | | 822 SEQ ID | |
| | or Clostridium difficile or a virus selected from rotavirus, | | NO: 12 | |
| | RSV, HIV, norvovirus, adenovirus, and astrovirus | | | |
| BACG51 | Light chain, Antibody against E. coli, Shigaella, | Ab 2 | US201200294822 | 22502 |
| | Entaamoeba histolvtica, Salmonella, Campylobacter, | | SEQ ID | |
| | or Clostridium difficile or a virus selected from rotavirus, | | NO: 14 | |
| | RSV, HIV, norvovirus, adenovirus, and astrovirus | | | |
| BACG52 | Light chain, Antibody against E. coli, Shigaella, | Ab 3 | US201200294822 | 22503 |
| | Entaamoeba histolvtica, Salmonella, Campylobacter, | | SEQ ID | |
| | or Clostridium difficile or a virus selected from rotavirus, | | NO: 22 | |
| | RSV, HIV, norvovirus, adenovirus, and astrovirus | | | |
| BACG53 | Light chain, Antibody against E. coli, Shigaella, | Ab 3 | US201200294822 | 22504 |
| | Entaamoeba histolvtica, Salmonella, Campylobacter, | | SEQ ID | |
| | or Clostridium difficile or a virus selected from rotavirus, | | NO: 24 | |
| | RSV, HIV, norvovirus, adenovirus, and astrovirus | | | |
| BACG54 | Light chain, Antibody against E. coli, Shigaella, | Ab 4 | US201200294822 | 22505 |
| | Entaamoeba histolvtica, Salmonella, Campylobacter, | | SEQ ID | |
| | or Clostridium difficile or a virus selected from rotavirus, | | NO: 32 | |
| | RSV, HIV, novovirus, adenovirus, and astrovirus | | | |
| BACG55 | Light chain, Antibody against E. coli, Shigaella, | Ab 4 | US201200294 | 22506 |
| | Entaamoeba histolvtica, Salmonella, Campylobacter, | | 822 SEQ ID | |
| | or Clostridium difficile or a virus selected from rotavirus, | | NO: 34 | |
| | RSV, HIV, norvovirus, adenovirus, and astrovirus | | | |
| BACG56 | Light chain, Antibody against E. coli, Shigaella, | Ab 5 | US201200294822 | 22507 |
| | Entaamoeba histolvtica, Salmonella, Campylobacter, | | SEQ ID | |
| | or Clostridium difficile or a virus selected from rotavirus, | | NO: 42 | |
| | RSV, HIV, norvovirus, adenovirus, and astrovirus | | | |
| BACG57 | Light chain, Antibody against E. coli, Shigaella, | Ab 5 | US201200294822 | 22508 |
| | Entaamoeba histolvtica, Salmonella, Campylobacter, | | SEQ ID | |
| | or Clostridium difficile or a virus selected from rotavirus, | | NO: 44 | |
| | RSV, HIV, norvovirus, adenovirus, and astrovirus | | | |
| BACG58 | Light chain, Antibody against E. coli, Shigaella, | Ab 6 | US201200294822 | 22509 |
| | Entaamoeba histolvtica, Salmonella, Campylobacter, | | SEQ ID | |
| | or Clostridium difficile or a virus selected from rotavirus, | | NO: 52 | |
| | RSV, HIV, norvovirus, adenovirus, and astrovirus | | | |
| BACG59 | Light chain, Antibody against E. coli, Shigaella, | Ab 6 | US201200294822 | 22510 |
| | Entaamoeba histolvtica, Salmonella, Campylobacter, | | SEQ ID | |
| | or Clostridium difficile or a virus selected from rotavirus, | | NO: 54 | |
| | RSV, HIV, norvovirus, adenovirus, and astrovirus | | | |
| BACG60 | Light chain, Antibody against E. coli, Shigaella, | Ab 7 | US201200294822 | 22511 |
| | Entaamoeba histolvtica, Salmonella, Campylobacter, | | SEQ ID | |
| | or Clostridium difficile or a virus selected from rotavirus, | | NO: 62 | |
| | RSV, HIV, norvovirus, adenovirus, and astrovirus | | | |
| BACG61 | Light chain, Antibody against E. coli, Shigaella, | Ab 7 | US201200294822 | 22512 |
| | Entaamoeba histolvtica, Salmonella, Campylobacter, | | SEQ ID | |
| | or Clostridium difficile or a virus selected from rotavirus, | | NO: 64 | |
| | RSV, HIV, norvovirus, adenovirus, and astrovirus | | | |
| BACG62 | Light chain, Antibody against E. coli, Shigaella, | Ab 8 | US201200294822 | 22513 |
| | Entaamoeba histolvtica, Salmonella, Campylobacter, | | SEQ ID | |
| | or Clostridium difficile or a virus selected from rotavirus, | | NO: 72 | |
| | RSV, HIV, norvovirus, adenovirus, and astrovirus | | | |
| BACG63 | Light chain, Antibody against E. coli, Shigaella, | Ab 8 | US201200294822 | 22514 |
| | Entaamoeba histolvtica, Salmonella, Campylobacter, | | SEQ ID | |
| | or Clostridium difficile or a virus selected from rotavirus, | | NO: 74 | |
| | RSV, HIV, norvovirus, adenovirus, and astrovirus | | | |
| BACG64 | Light chain, Antibody against E. coli, Shigaella, | Ab 9 | US201200294822 | 22515 |
| | Entaamoeba histolvtica, Salmonella, Campylobacter, | | SEQ ID | |
| | or Clostridium difficile or a virus selected from rotavirus, | | NO: 82 | |
| | RSV, HIV, norvovirus, adenovirus, and astrovirus | | | |
| BACG65 | Light chain, Antibody against E. coli, Shigaella, | Ab 9 | US201200294822 | 22516 |
| | Entaamoeba histolvtica, Salmonella, Campylobacter, | | SEQ ID | |
| | or Clostridium difficile or a virus selected from rotavirus, | | NO: 84 | |
| | RSV, HIV, norvovirus, adenovirus, and astrovirus | | | |
| BACG66 | Light chain, Antibody against E. coli, Shigaella, | Ab 10 | US201200294822 | 22517 |
| | Entaamoeba histolvtica, Salmonella, Campylobacter, | | SEQ ID | |
| | or Clostridium difficile or a virus selected from rotavirus, | | NO: 92 | |
| | RSV, HIV, norvovirus, adenovirus, and astrovirus | | | |
| BACG67 | Light chain, Antibody against E. coli, Shigaella, | Ab 10 | US201200294822 | 22518 |
| | Entaamoeba histolvtica, Salmonella, Campylobacter, | | SEQ ID | |
| | or Clostridium difficile or a virus selected from rotavirus, | | NO: 94 | |
| | RSV, HIV, norvovirus, adenovirus, and astrovirus | | | |
| BACG68 | Light chain, Antibody against E. coli, Shigaella, | Ab 11 | US201200294822 | 22519 |
| | Entaamoeba histolvtica, Salmonella, Campylobacter, | | SEQ ID | |
| | or Clostridium difficile or a virus selected from rotavirus, | | NO: 102 | |
| | RSV, HIV, norvovirus, adenovirus, and astrovirus | | | |
| BACG69 | Light chain, Antibody against E. coli, Shigaella, | Ab 11 | US201200294822 | 22520 |
| | Entaamoeba histolvtica, Salmonella, Campylobacter, | | SEQ ID | |
| | or Clostridium difficile or a virus selected from rotavirus, | | NO: 104 | |
| | RSV, HIV, norvovirus, adenovirus, and astrovirus | | | |
| BACG70 | Light chain, Antibody against E. coli, Shigaella, | Ab 12 | US201200294822 | 22521 |
| | Entaamoeba histolvtica, Salmonella, Campylobacter, | | SEQ ID | |
| | or Clostridium difficile or a virus selected from rotavirus, | | NO: 112 | |
| | RSV, HIV, norvovirus, adenovirus, and astrovirus | | | |
| BACG71 | Light chain, Antibody against E. coli, Shigaella, | Ab 12 | US201200294822 | 22522 |
| | Entaamoeba histolvtica, Salmonella, Campylobacter, | | SEQ ID | |
| | or Clostridium difficile or a virus selected from rotavirus, | | NO: 114 | |
| | RSV, HIV, norvovirus, adenovirus, and astrovirus | | | |
| BACG72 | Light chain, Antibody against E. coli, Shigaella, | Ab 13 | US201200294822 | 22523 |
| | Entaamoeba histolvtica, Salmonella, Campylobacter, | | SEQ ID | |
| | or Clostridium difficile or a virus selected from rotavirus, | | NO: 122 | |
| | RSV, HIV, norvovirus, adenovirus, and astrovirus | | | |
| BACG73 | Light chain, Antibody against E. coli, Shigaella, | Ab 13 | US201200294822 | 22524 |
| | Entaamoeba histolvtica, Salmonella, Campylobacter, | | SEQ ID | |
| | or Clostridium difficile or a virus selected from rotavirus, | | NO: 124 | |
| | RSV, HIV, norvovirus, adenovirus, and astrovirus | | | |
| BACG74 | Light chain, Antibody against E. coli, Shigaella, | Ab 14 | US201200294822 | 22525 |
| | Entaamoeba histolvtica, Salmonella, Campylobacter, | | SEQ ID | |
| | or Clostridium difficile or a virus selected from rotavirus, | | NO: 132 | |
| | RSV, HIV, norvovirus, adenovirus, and astrovirus | | | |
| BACG75 | Light chain, Antibody against E. coli, Shigaella, | Ab 14 | US201200294822 | 22526 |
| | Entaamoeba histolvtica, Salmonella, Campylobacter, | | SEQ ID | |
| | or Clostridium difficile or a virus selected from rotavirus, | | NO: 134 | |
| | RSV, HIV, norvovirus, adenovirus, and astrovirus | | | |
| BACG76 | Light chain, Antibody against E coli, Shigella, | Ab2 | WO2012162253 | 22527 |
| | Entaamoeba histolytica, Salmonella, Campylobacter, or | | SEQ ID | |
| | Clostridium difficile, rotavirus, RSV, HIV, norvovirus, | | NO: 11 | |
| | adenovirus, and astrovirus, other diseases causing | | | |
| | diarrhea | | | |
| BACG77 | Light chain, Antibody against E coli, Shigella, | Ab3 | WO2012162253 | 22528 |
| | Entaamoeba histolytica, Salmonella, Campylobacter, or | | SEQ ID | |
| | Clostridium difficile, rotavirus, RSV, HIV, norvovirus, | | NO: 22 | |
| | adenovirus, and astrovirus, other diseases causing | | | |
| | diarrhea | | | |
| BACG78 | Light chain, Antibody against E coli, Shigella, | Ab4 | WO2012162253 | 22529 |
| | Entaamoeba histolytica, Salmonella, Campylobacter, or | | SEQ ID | |
| | Clostridium difficile, rotavirus, RSV, HIV, norvovirus, | | NO: 31 | |
| | adenovirus, and astrovirus, other diseases causing | | | |
| | diarrhea | | | |
| BACG79 | Light chain, Antibody against E coli, Shigella, | Ab5 | WO2012162253 | 22530 |
| | Entaamoeba histolytica, Salmonella, Campylobacter, or | | SEQ ID | |
| | Clostridium difficile, rotavirus, RSV, HIV, norvovirus, | | NO: 42 | |
| | adenovirus, and astrovirus, other diseases causing | | | |
| | diarrhea | | | |
| BACG80 | Light chain, Antibody against E coli, Shigella, | Ab6 | WO2012162253 | 22531 |
| | Entaamoeba histolytica, Salmonella, Campylobacter, or | | SEQ ID | |
| | Clostridium difficile, rotavirus, RSV, HIV, norvovirus, | | NO: 52 | |
| | adenovirus, and astrovirus, other diseases causing | | | |
| | diarrhea | | | |
| BACG81 | Light chain, Antibody against E coli, Shigella, | Ab7 | WO2012162253 | 22532 |
| | Entaamoeba histolytica, Salmonella, Campylobacter, or | | SEQ ID | |
| | Clostridium difficile, rotavirus, RSV, HIV, norvovirus, | | NO: 61 | |
| | adenovirus, and astrovirus, other diseases causing | | | |
| | diarrhea | | | |
| BACG82 | Light chain, Antibody against E coli, Shigella, | Ab8 | WO2012162253 | 22533 |
| | Entaamoeba histolytica, Salmonella, Campylobacter, or | | SEQ ID | |
| | Clostridium difficile, rotavirus, RSV, HIV, norvovirus, | | NO: 71 | |
| | adenovirus, and astrovirus, other diseases causing | | | |
| | diarrhea | | | |
| BACG83 | Light chain, Antibody against E coli, Shigella, | Ab9 | WO2012162253 | 22534 |
| | Entaamoeba histolytica, Salmonella, Campylobacter, or | | SEQ ID | |
| | Clostridium difficile, rotavirus, RSV, HIV, norvovirus, | | NO: 82 | |
| | adenovirus, and astrovirus, other diseases causing | | | |
| | diarrhea | | | |
| BACG84 | Light chain, Antibody against E coli, Shigella, | Ab10 | WO2012162253 | 22535 |
| | Entaamoeba histolytica, Salmonella, Campylobacter, or | | SEQ ID | |
| | Clostridium difficile, rotavirus, RSV, HIV, norvovirus, | | NO: 91 | |
| | adenovirus, and astrovirus, other diseases causing | | | |
| | diarrhea | | | |
| BACG85 | Light chain, Antibody against E coli, Shigella, | Ab11 | WO2012162253 | 22536 |
| | Entaamoeba histolytica, Salmonella, Campylobacter, or | | SEQ ID | |
| | Clostridium difficile, rotavirus, RSV, HIV, norvovirus, | | NO: 102 | |
| | adenovirus, and astrovirus, other diseases causing | | | |
| | diarrhea | | | |
| BACG86 | Light chain, Antibody against E coli, Shigella, | Ab12 | WO2012162253 | 22537 |
| | Entaamoeba histolytica, Salmonella, Campylobacter, or | | SEQ ID | |
| | Clostridium difficile, rotavirus, RSV, HIV, norvovirus, | | NO: 112 | |
| | adenovirus, and astrovirus, other diseases causing | | | |
| | diarrhea | | | |
| BACG87 | Light chain, Antibody against E coli, Shigella, | Ab13 | WO2012162253 | 22538 |
| | Entaamoeba histolytica, Salmonella, Campylobacter, or | | SEQ ID | |
| | Clostridium difficile, rotavirus, RSV, HIV, norvovirus, | | NO: 122 | |
| | adenovirus, and astrovirus, other diseases causing | | | |
| | diarrhea | | | |
| BACG88 | Light chain, Antibody against E coli, Shigella, | Ab14 | WO2012162253 | 22539 |
| | Entaamoeba histolytica, Salmonella, Campylobacter, or | | SEQ ID | |
| | Clostridium difficile, rotavirus, RSV, HIV, norvovirus, | | NO: 132 | |
| | adenovirus, and astrovirus, other diseases causing | | | |
| | diarrhea | | | |
| BACG89 | Light chain, Antibody against Escherichia coli infection, | | WO2014070117 | 22540 |
| | Staphylococcus infection | | SEQ ID | |
| | | | NO: 4 | |
| BACG90 | Light chain, Antibody against Listeria monocytogenes or | 6H8 | U.S. Pat. No. | 22541 |
| | WR-tubercle bacillus | | 8,445,643 | |
| | | | SEQ ID NO: 6 | |
| BACG91 | Light chain, Staphylococcus enterotoxin B | F10 | U.S. Pat. No. | 22542 |
| | | | 8,895,704 | |
| | | | SEQ ID NO: 28 | |
| BACG92 | Light chain, Staphylococcus enterotoxin B | 100C9 | U.S. Pat. No. | 22543 |
| | | | 8,895,704 | |
| | | | SEQ ID NO: 32 | |
| BACG93 | Light chain, Staphylococcus enterotoxin B | 79G9 | U.S. Pat. No. | 22544 |
| | | | 8895704 | |
| | | | SEQ ID NO: 36 | |
| BACG94 | Light chain, Staphylococcus enterotoxin B | 154G12 | U.S. Pat. No. | 22545 |
| | | | 8,895,704 | |
| | | | SEQ ID NO: 134 | |
| BACG95 | ScFv, Antibody against Clostridium perfringens, anti- | ScFv-1A8 | Zhao, B. and | 22546 |
| | alpha toxin 1A8 | | Xu, C. | |
| | | | “Cloning and | |
| | | | sequencing of | |
| | | | the ScFv-2.E3 | |
| | | | gene anti- | |
| | | | alpha toxin of | |
| | | | clostridium | |
| | | | perfringens | |
| | | | type A”, Chin. | |
| | | | J. Vet. Sci. 20, | |
| | | | 246-248 | |
| | | | (2000), CNBI | |
| | | | Accession # | |
| | | | AAU11282 | |
| BACG96 | ScFv, Antibody against Clostridium perfringens, anti- | ScFv-2E3 | Zhao, B. and | 22547 |
| | alpha toxin 2E3 | | Xu, C. | |
| | | | “Cloning and | |
| | | | sequencing of | |
| | | | the ScFv-2E3 | |
| | | | gene anti- | |
| | | | alpha toxin of | |
| | | | clostridium | |
| | | | perfringens | |
| | | | type A”, Chin. | |
| | | | J. Vet. Sci. 20, | |
| | | | 246-248 | |
| | | | (2000), NCBI | |
| | | | Accession # | |
| | | | AAU11283 | |
| BACG97 | Variable fragment, Antibody against Pseudomonas, | αTT2 | U.S. Pat. No. | 22548 |
| | Clostridium, Staphylococcus, Pasteurella, Yersinia, | | 7,655,759 | |
| | Bacillus anthracis, Neisseria, Vibrio, enterotoxic E. coli, | | SEQ ID NO: 8 | |
| | Salmonella, Shigella, and Listeria, Clostridium, | | | |
| | Staphylococcus, Pseudomonas, Pasteurella, Yersinia, | | | |
| | Bacillus anthracis, Neisseria, Vibrio, enterotoxic E. coli, | | | |
| | Salmonella, Shigella, and Listeria bacteria | | | |
| BACG98 | Variable fragment, antibody against Pseudomonas, | αTT1 | U.S. Pat. No. | 22549 |
| | Clostridium, Staphylococcus, Pasteurella, Yersinia, | | 7,655,759 | |
| | Bacillus anthracis, Neisseria, Vibrio, enterotoxic E. coli, | | SEQ ID NO: 7 | |
| | Salmonella, Shigella, and Listeria, Clostridium, | | | |
| | Staphylococcus, Pseudomonas,Pasteurella, Yersinia, | | | |
| | Bacillus anthracis, Neisseria, Vibrio, enterotoxic E. coli, | | | |
| | Salmonella, Shigella, and Listeria bacteria, | | | |

In some embodiments, the payload region of the viral particle comprises one or more nucleic acid sequences encoding one or more of the payload antibody polypeptides listed in Table 17 against Hepatitis A and/or Hepatitis E (HEPAE1-HEPAE41; SEQ ID NO: 22550-22590).

TABLE 17
Antibodies against Hepatitis A and Hepatitis E

| Antibody | | | | SEQ |
|---|---|---|---|---|
| No. | Description | Antibody Name | Reference Information | ID NO |
| HEPAE1 | Heavy chain variable region, | HEV-Fab-216 | CN1486990A; CN100497391C | 22550 |
| | HEV Ab, a humanized | | | |
| | neutralizing genetically | | | |
| | engineered antibody | | | |
| HEPAE2 | Heavy chain variable region, | HEV-Fab-315 | CN1486990A; CN100497391C | 22551 |
| | HEV Ab, a humanized | | | |
| | neutralizing genetically | | | |
| | engineered antibody | | | |
| HEPAE3 | Heavy chain variable region, | HEV-Fab-319 | CN1486990A; CN100497391C | 22552 |
| | HEV Ab, a humanized | | | |
| | neutralizing genetically | | | |
| | engineered antibody | | | |
| HEPAE4 | Heavy chain variable region, | HEV-Fab-328 | CN1486990A; CN100497391C | 22553 |
| | HEV Ab, a humanized | | | |
| | neutralizing genetically | | | |
| | engineered antibody | | | |
| HEPAE5 | Heavy chain variable region, | HEV-Fab-404 | CN1486990A; CN100497391C | 22554 |
| | HEV Ab, a humanized | | | |
| | neutralizing genetically | | | |
| | engineered antibody | | | |
| HEPAE6 | Heavy chain variable region, | 13D8 | U.S. Pat. No. 7,786,264 SEQ ID NO. 8; | 22555 |
| | HEV monoclonal antibody | | US20060233822; US20100003281; | |
| | | | EP1452541; EP2322625 | |
| HEPAE7 | Heavy chain variable region, | 16D7 | U.S. Pat. No. 7,786,264 SEQ ID NO. 20; | 22556 |
| | HEV monoclonal antibody | | US20060233822; US20100003281; | |
| | | | EP1452541; EP2322625 | |
| HEPAE8 | Heavy chain variable region, | 8C11 | U.S. Pat. No. 7,786,264 SEQ ID NO. 12; | 22557 |
| | HEV monoclonal antibody | | US20060233822; US20100003281; | |
| | | | EP1452541; EP2322625 | |
| HEPAE9 | Heavy chain variable region, | 8H3 | U.S. Pat. No. 7,786,264 SEQ ID NO. 16; | 22558 |
| | HEV monoclonal antibody | | US20060233822; US20100003281; | |
| | | | EP1452541; EP2322625 | |
| HEPAE10 | Heavy chain variable region, | HEV#31 | U.S. Pat. No. 7,148,323 SEQ ID NO: 3; | 22559 |
| | HEV neutralizing antibody | | US20050233316; U.S. Pat. No. 6,930,176; | |
| | | | WO2001040270 | |
| HEPAE11 | Heavy chain variable region, | HEV#4 | U.S. Pat. No. 7,786,264 SEQ ID NO: 1; | 22560 |
| | HEV neutralizing antibody | | US20050233316; U.S. Pat. No. 6,930,176; | |
| | | | WO2001040270 | |
| HEPAE12 | Heavy chain variable region, | anti-HAV | Kim S. J., et al., Neutralizing | 22561 |
| | partial, HAV | capsid | human monoclonal antibodies to | |
| | | | hepatitis A virus recovered by | |
| | | | phage display; Virology 318 (2), | |
| | | | 598-607 (2004), NCBI Accession | |
| | | | # AAO86899.1(124aa) | |
| HEPAE13 | Heavy chain variable region, | anti-HAV | Kim S. J., et al., Neutralizing | 22562 |
| | partial, HAV | capsid | human monoclonal antibodies to | |
| | | | hepatitis A virus recovered by | |
| | | | phage display; Virology 318 (2), | |
| | | | 598-607 (2004), NCBI Accession | |
| | | | # AAO86898.1(129aa) | |
| HEPAE14 | Heavy chain variable region, | anti-HAV | Kim S. J., et al., Neutralizing | 22563 |
| | partial, HAV | capsid | human monoclonal antibodies to | |
| | | | hepatitis A virus recovered by | |
| | | | phage display; Virology 318 (2), | |
| | | | 598-607 (2004), NCBI Accession | |
| | | | # AAO86897.1(123aa) | |
| HEPAE15 | Heavy chain variable region, | anti-HA V | Kim S. J., et al., Neutralizing | 22564 |
| | partial, HAV | capsid | human monoclonal antibodies to | |
| | | | hepatitis A virus recovered by | |
| | | | phage display; Virology 318 (2), | |
| | | | 598-607 (2004), NCBI Accession | |
| | | | # AAO86896.1(129aa) | |
| HEPAE16 | Heavy chain, HEV antibody | 8g12 | Gu Y., et al., Structural basis for | 22565 |
| | (mouse monoclonal antibody), | | the neutralization of hepatitis E | |
| | E2 glycoprotein | | virus by a cross-genotype | |
| | | | antibody; Cell Res. 25 (5), | |
| | | | 604-620 (2015); NCBI Accession | |
| | | | # 4PLJ_H (229aa) | |
| HEPAE17 | Heavy chain, HEV antibody | | Tang X., et al., Proc. Natl. Acad. | 22566 |
| | (mouse monoclonal antibody), E2 | | Sci. U.S.A. 108 (25), 10266- | |
| | glycoprotein | | 10271 (2011); NCBI Accession # | |
| | | | 3RKD_H(230aa) | |
| HEPAE18 | Light chain variable region, | HAV#14 | U.S. Pat. No. 7,635,476 SEQ ID NO: 4; | 22567 |
| | gamma1, HAV, | | U.S. Pat. No. 7,282,205; US20040260067; | |
| | | | US20070287667; WO2003040341 | |
| HEPAE19 | Light chain variable region, | HAV#4 | U.S. Pat. No. 7,635,476 SEQ ID NO: 1; | 22568 |
| | gamma1, HAV, | | U.S. Pat. No. 7,282,205; US20040260067; | |
| | | | US20070287667; WO2003040341 | |
| HEPAE20 | Light chain variable region, | HAV#5 | U.S. Pat. No. 7,635,476 SEQ ID NO: 2; | 22569 |
| | gamma1, HAV, | | U.S. Pat. No. 7,282,205; US20040260067; | |
| | | | US20070287667; WO2003040341 | |
| HEPAE21 | Light chain variable region, | HAV#6 | U.S. Pat. No. 7,635,476 SEQ ID NO: 3; | 22570 |
| | gamma1, HAV, | | U.S. Pat. No. 7,282,205; US20040260067; | |
| | | | US20070287667; WO2003040341 | |
| HEPAE22 | Light chain variable region, HEV | HEV-Fab-216 | CN1486990A; CN100497391C | 22571 |
| | Ab, a humanized neutralizing | | | |
| | genetically engineered antibody | | | |
| HEPAE23 | Light chain variable region, HEV | HEV-Fab-315 | CN1486990A; CN100497391C | 22572 |
| | Ab, a humanized neutralizing | | | |
| | genetically engineered antibody | | | |
| HEPAE24 | Light chain variable region, HEV | HEV-Fab-319 | CN1486990A; CN100497391C | 22573 |
| | Ab, a humanized neutralizing | | | |
| | genetically engineered antibody | | | |
| HEPAE25 | Light chain variable region, HEV | HEV-Fab-328 | CN1486990A; CN100497391C | 22574 |
| | Ab, a humanized neutralizing | | | |
| | genetically engineered antibody | | | |
| HEPAE26 | Light chain variable region, HEV | HEV-Fab-404 | CN1486990A; CN100497391C | 22575 |
| | Ab, a humanized neutralizing | | | |
| | genetically engineered antibody | | | |
| HEPAE27 | Light chain variable region, | | WO2011114353 SEQ ID NO: 25 | 22576 |
| | monovalent, HAV | | | |
| HEPAE28 | Light chain variable region, | anti-HAV | Kim S. J., et al., Neutralizing | 22577 |
| | partial, HAV | capsid | human monoclonal antibodies to | |
| | | | hepatitis A virus recovered by | |
| | | | phage display; Virology 318 (2), | |
| | | | 598-607 (2004), NCBI Accession | |
| | | | # AAO86903.1(107aa) | |
| HEPAE29 | Light chain variable region, | anti-HAV | Kim S. J., el al., Neutralizing | 22578 |
| | partial, HAV | capsid | human monoclonal antibodies to | |
| | | | hepatitis A virus recovered by | |
| | | | phage display; Virology 318 (2), | |
| | | | 598-607 (2004), NCBI Accession | |
| | | | # AAO86902.1(107aa) | |
| HEPAE30 | Light chain variable region, | anti-HAV | Kim S. J., el al., Neutralizing | 22579 |
| | partial, HAV | capsid | human monoclonal antibodies to | |
| | | | hepatitis A virus recovered by | |
| | | | phage display; Virology 318 (2), | |
| | | | 598-607 (2004), NCBI Accession | |
| | | | # AAO86901.1(107aa) | 22580 |
| HEPAE31 | Light chain variable region, | anti-HAV | Kim S. J., et al., Neutralizing | |
| | partial, HAV | capsid | human monoclonal antibodies to | |
| | | | hepatitis A virus recovered by | |
| | | | phage display; Virology 318 (2), | |
| | | | 598-607 (2004). NCBI Accession | |
| | | | # AAO86900.1(107aa) | |
| HEPAE32 | Light chain variable, HEV | 13D8 | U.S. Pat. No. 7,786,264 SEQ ID NO. 6; | 22581 |
| | monoclonal antibody | | US20060233822; US20100003281; | |
| | | | EP1452541; EP2322625 | |
| HEPAE33 | Light chain variable, HEV | 16D7 | U.S. Pat. No. 7,786,264 SEQ ID NO. 18; | 22582 |
| | monoclonal antibody | | US20060233822; US20100003281; | |
| | | | EP1452541; EP2322625 | |
| HEPAE34 | Light chain variable, HEV | 8C11 | U.S. Pat. No. 7,786,264 SEQ ID NO: 10; | 22583 |
| | monoclonal antibody | | US20060233822; US20100003281; | |
| | | | EP1452541; EP2322625 | |
| HEPAE35 | Light chain variable, HEV | 8H3 | U.S. Pat. No. 7,786,264 SEQ ID NO. 14; | 22584 |
| | monoclonal antibody | | US20060233822; US20100003281; | |
| | | | EP1452541; EP2322625 | |
| HEPAE36 | Light chain variable, HEV | HEV#31 | U.S. Pat. No. 7,148,323 SEQ ID NO: 4; | 22585 |
| | monoclonal antibody | | US20050233316; U.S. Pat. No. 6,930,176; | |
| | | | WO2001040270 | |
| HEPAE37 | Light chain variable, HEV | HEV#4 | U.S. Pat. No. 7,148,323 SEQ ID NO: 2; | 22586 |
| | monoclonal antibody | | US20050233316; U.S. Pat. No. 6,930,176; | |
| | | | WO2001040270 | |
| HEPAE38 | Light chain, E2 glycoprotein, | 8g12 | Gu Y., et al., Structural basis for | 22587 |
| | HEV antibody (mouse | | the neutralization of hepatitis E | |
| | monoclonal antibody) | | virus by a cross-genotype | |
| | | | antibody; Cell Res. 25 (5), 604- | |
| | | | 620 (2015); NCBI Accession # | |
| | | | 4PLJ_L (212aa) | |
| HEPAE39 | Light chain, E2 glycoprotein, | | Tang X., et al., Proc. Natl. Acad. | 22588 |
| | HEV antibody (mouse | | Sci. U.S.A. 108 (25), 10266- | |
| | monoclonal antibody) | | 10271 (2011), NCBI Accession # | |
| | | | 3RKD_C (214aa) | |
| HEPAE40 | Monovalent Heavy chain variable | | WO2011114353 SEQ ID NO: 24 | 22589 |
| | region, HAV | | | |
| HEPAE41 | ScFv, HAV, Monovalent human | | WO2011114353 SEQ ID NO: 27 | 22590 |
| | antibody | | | |

In some embodiments, the payload region of the viral particle comprises one or more nucleic acid sequences, fragment or variants thereof or encodes one or more polypeptides, fragments or variants thereof described in Chinese Pub. No. CN103923881, CN103923882, CN1605628, CN1318565, CN1163512, the contents of each of which are herein incorporated by reference in their entirety, against HAV.

In some embodiments, the payload region of the viral particle comprises one or more nucleic acid sequences encoding one or more of the payload antibody polypeptides listed in Table 18 against Norwalk virus (NORV1-NORV48; SEQ ID NO: 22591-22638).

TABLE 18
Antibodies against Norwalk virus

| Antibody | | Antibody | | SEQ |
|---|---|---|---|---|
| No. | Description | Name | Reference Information | ID NO |
| NORV1 | Heavy chain variable region, | B7 | WO2014126921 SEQ ID NO: 8 | 22591 |
| | Norwalk virus | | | |
| NORV2 | Light chain variable region, | B7 | WO2014126921 SEQ ID NO: 16 | 22592 |
| | Norwalk virus | | | |
| NORV3 | Heavy chain variable region, | B72 | WO2014126921 SEQ ID NO: 120 | 22593 |
| | Norwalk virus | | | |
| NORV4 | Light chain variable region, | B72 | WO2014126921 SEQ ID NO: 128 | 22594 |
| | Norwalk virus | | | |
| NORV5 | Heavy chain variable region, | C9 | WO2014126921 SEQ ID NO: 88 | 22595 |
| | Norwalk virus | | | |
| NORV6 | Light chain variable region, | C9 | WO2014126921 SEQ ID NO: 96 | 22596 |
| | Norwalk virus | | | |
| NORV7 | Heavy chain variable region, | D4 | WO2014126921 SEQ ID NO: 136 | 22597 |
| | Norwalk virus | | | |
| NORV8 | Light chain variable region, | D4 | WO2014126921 SEQ ID NO: 144 | 22598 |
| | Norwalk virus | | | |
| NORV9 | Heavy chain variable region, | D8 | WO2014126921 SEQ ID NO: 24 | 22599 |
| | Norwalk virus | | | |
| NORV10 | Light chain variable region, | D8 | WO2014126921 SEQ ID NO: 32 | 22600 |
| | Norwalk virus | | | |
| NORV11 | Heavy chain variable region, | E5 | WO2014126921 SEQ ID NO: 40 | 22601 |
| | Norwalk virus | | | |
| NORV12 | Light chain variable region, | E5 | WO2014126921 SEQ ID NO: 48 | 22602 |
| | Norwalk virus | | | |
| NORV13 | Heavy chain variable region, | FI1 | WO2014126921 SEQ ID NO: 72 | 22603 |
| | Norwalk virus | | | |
| NORV14 | Light chain variable region, | FI1 | WO2014126921 SEQ ID NO: 80 | 22604 |
| | Norwalk virus | | | |
| NORV15 | Heavy chain variable region, | G3 | WO2014126921 SEQ ID NO: 104 | 22605 |
| | Norwalk virus | | | |
| NORV16 | Light chain variable region, | G3 | WO2014126921 SEQ ID NO: 112 | 22606 |
| | Norwalk virus | | | |
| NORV17 | Heavy chain variable region, | G4 | WO2014126921 SEQ ID NO: 56 | 22607 |
| | Norwalk virus | | | |
| NORV18 | Light chain variable region, | G4 | WO2014126921 SEQ ID NO: 64 | 22608 |
| | Norwalk virus | | | |
| NORV19 | Heavy chain variable region, | | WO2014183052 SEQ ID NO: 1 | 22609 |
| | Norwalk or MD2004 virus | | | |
| NORV20 | Heavy chain variable region, | | WO2014183052 SEQ ID NO: 2 | 22610 |
| | Norwalk or MD2004 virus | | | |
| NORV21 | Heavy chain variable region, | | WO2014183052 SEQ ID NO: 3 | 22611 |
| | Norwalk or MD2004 virus | | | |
| NORV22 | Heavy chain variable region, | | WO2014183052 SEQ ID NO: 4 | 22612 |
| | Norwalk or MD2004 virus | | | |
| NORV23 | Heavy chain variable region, | | WO2014183052 SEQ ID NO: 5 | 22613 |
| | Norwalk or MD2004 virus | | | |
| NORV24 | Heavy chain variable region, | | WO2014183052 SEQ ID NO: 6 | 22614 |
| | Norwalk or MD2004 virus | | | |
| NORV25 | Heavy chain variable region, | | WO2014183052 SEQ ID NO: 7 | 22615 |
| | Norwalk or MD2004 virus | | | |
| NORV26 | Heavy chain variable region, | | WO2014183052 SEQ ID NO: 8 | 22616 |
| | Norwalk or MD2004 virus | | | |
| NORV27 | Heavy chain variable region, | | WO2014183052 SEQ ID NO: 9 | 22617 |
| | Norwalk or MD2004 virus | | | |
| NORV28 | Heavy chain variable region, | | WO2014183052 SEQ ID NO: 10 | 22618 |
| | Norwalk or MD2004 virus | | | |
| NORV29 | Heavy chain variable region, | | WO2014183052 SEQ ID NO: 11 | 22619 |
| | Norwalk or MD2004 virus | | | |
| NORV30 | Heavy chain variable region, | | WO2014183052 SEQ ID NO: 12 | 22620 |
| | Norwalk or MD2004 virus | | | |
| NORV31 | Heavy chain variable region, | | WO2014183052 SEQ ID NO: 13 | 22621 |
| | Norwalk or MD2004 virus | | | |
| NORV32 | Heavy chain variable region, | | WO2014183052 SEQ ID NO: 14 | 22622 |
| | Norwalk or MD2004 virus | | | |
| NORV33 | Heavy chain variable region, | | WO2014183052 SEQ ID NO: 15 | 22623 |
| | Norwalk or MD2004 virus | | | |
| NORV34 | Heavy chain variable region, | | WO2014183052 SEQ ID NO: 16 | 22624 |
| | Norwalk or MD2004 virus | | | |
| NORV35 | Heavy chain variable region, | | WO2014183052 SEQ ID NO: 17 | 22625 |
| | Norwalk or MD2004 virus | | | |
| NORV36 | Heavy chain variable region, | | WO2014183052 SEQ ID NO: 18 | 22626 |
| | Norwalk or MD2004 virus | | | |
| NORV37 | Heavy chain variable region, | | WO2014183052 SEQ ID NO: 19 | 22627 |
| | Norwalk or MD2004 virus | | | |
| NORV38 | Heavy chain variable region, | | WO2014183052 SEQ ID NO: 20 | 22628 |
| | Norwalk or MD2004 virus | | | |
| NORV39 | Heavy chain variable region, | | WO2014183052 SEQ ID NO: 21 | 22629 |
| | Norwalk or MD2004 virus | | | |
| NORV40 | Heavy chain variable region, | | WO2014183052 SEQ ID NO: 22 | 22630 |
| | Norwalk or MD2004 virus | | | |
| NORV41 | Heavy chain variable region, | | WO2014183052 SEQ ID NO: 23 | 22631 |
| | Norwalk or MD2004 virus | | | |
| NORV42 | Heavy chain variable region, | | WO2014183052 SEQ ID NO: 24 | 22632 |
| | Norwalk or MD2004 virus | | | |
| NORV43 | Heavy chain variable region, | | WO2014183052 SEQ ID NO: 25 | 22633 |
| | Norwalk or MD2004 virus | | | |
| NORV44 | Heavy chain variable region, | | WO2014183052 SEQ ID NO: 26 | 22634 |
| | Norwalk or MD2004 virus | | | |
| NORV45 | Heavy chain variable region, | | WO2014183052 SEQ ID NO: 27 | 22635 |
| | Norwalk or MD2004 virus | | | |
| NORV46 | Heavy chain variable region, | | WO2014183052 SEQ ID NO: 28 | 22636 |
| | Norwalk or MD2004 virus | | | |
| NORV47 | Heavy chain variable region, | | WO2014183052 SEQ ID NO: 29 | 22637 |
| | Norwalk or MD2004 virus | | | |
| NORV48 | Heavy chain variable region, | | WO2014183052 SEQ ID NO: 30 | 22638 |
| | Norwalk or MD2004 virus | | | |

In some embodiments, the payload region of the viral particle comprises one or more nucleic acid sequences encoding one or more of the payload antibody polypeptides listed in Table 19 against Rotavirus (ROTV1-ROTV25; SEQ ID NO: 22639-22663).

TABLE 19
Antibodies against rotavirus

| Antibody | | | SEQ |
|---|---|---|---|
| No. | Description | Reference Information | ID NO |
| ROTV1 | Heavy chain single domain | U.S. Pat. No. 8,105,592; US20090226418 SEQ ID NO: 1 | 22639 |
| ROTV2 | Heavy chain single domain | U.S. Pat. No. 8,105,592; US20090226418 SEQ ID NO: 2 | 22640 |
| ROTV3 | Heavy chain single domain | U.S. Pat. No. 8,105,592; US20090226418 SEQ ID NO: 3 | 22641 |
| ROTV4 | Heavy chain single domain | U.S. Pat. No. 8,105,592; US20090226418 SEQ ID NO: 4 | 22642 |
| ROTV5 | Heavy chain single domain | U.S. Pat. No. 8,105,592; US20090226418 SEQ ID NO: 5 | 22643 |
| ROTV6 | Heavy chain single domain | U.S. Pat. No. 8,105,592; US20090226418 SEQ ID NO: 6 | 22644 |
| ROTV7 | Heavy chain single domain | U.S. Pat. No. 8,105,592; US20090226418 SEQ ID NO: 7 | 22645 |
| ROTV8 | Heavy chain single domain | U.S. Pat. No. 8,105,592; US20090226418 SEQ ID NO: 8 | 22646 |
| ROTV9 | Heavy chain single domain | U.S. Pat. No. 8,105,592; US20090226418 SEQ ID NO: 9 | 22647 |
| ROTV10 | Heavy chain single domain | U.S. Pat. No. 8,105,592; US20090226418 SEQ ID NO: 10 | 22648 |
| ROTV11 | Heavy chain single domain | U.S. Pat. No. 8,105,592; US20090226418 SEQ ID NO: 11 | 22649 |
| ROTV12 | Heavy chain single domain | U.S. Pat. No. 8,105,592; US20090226418 SEQ ID NO: 12 | 22650 |
| ROTV13 | Heavy chain single domain | U.S. Pat. No. 8,105,592; US20090226418 SEQ ID NO: 13 | 22651 |
| ROTV14 | Heavy chain single domain | U.S. Pat. No. 8,105,592; US20090226418 SEQ ID NO: 14 | 22652 |
| ROTV15 | Heavy chain single domain | U.S. Pat. No. 8,105,592; US20090226418 SEQ ID NO: 15 | 22653 |
| ROTV16 | Heavy chain single domain | U.S. Pat. No. 8,105,592; US20090226418 SEQ ID NO: 16 | 22654 |
| ROTV17 | Heavy chain single domain | U.S. Pat. No. 8,105,592; US20090226418 SEQ ID NO: 17 | 22655 |
| ROTV18 | Heavy chain single domain | U.S. Pat. No. 8,105,592; US20090226418 SEQ ID NO: 18 | 22656 |
| ROTV19 | Heavy chain single domain | U.S. Pat. No. 8,105,592; US20090226418 SEQ ID NO: 19 | 22657 |
| ROTV20 | Heavy chain single domain | U.S. Pat. No. 8,105,592; US20090226418 SEQ ID NO: 20 | 22658 |
| ROTV21 | Heavy chain single domain | U.S. Pat. No. 8,105,592; US20090226418 SEQ ID NO: 21 | 22659 |
| ROTV22 | Human VP6 polypeptide | US20030166139 SEQ ID NO: 2 | 22660 |
| ROTV23 | Human VP6 polypeptide | US20030166139 SEQ ID NO: 4 | 22661 |
| ROTV24 | | Aiyegbo, M. S., et al “Human RotavirUSVp6- | 22662 |
| | | Specific Antibodies Mediate intracellular | |
| | | Neutralization By Binding To A Quater | |
| | | Structure in The Transcriptional Pore”, Plos One | |
| | | 8, 61101 (2013), NCBI Accession # 4HFW_B | |
| ROTV25 | | Aiyegbo, M. S., et al “Human RotavirUSVp6- | 22663 |
| | | Specific Antibodies Mediate intracellular | |
| | | Neutralization By Binding To A Quater | |
| | | Structure in The Transcriptional Pore”, Plos One | |
| | | 8, 61101 (2013), NCBI Accession # 4HFW_A | |

In some embodiments, the payload region of the viral particle comprises one or more nucleic acid sequences encoding one or more of the payload antibody polypeptides listed in Table 20 against Entamoeba Histolytica (ENTH1-ENTH16; SEQ ID NO: 22664-22679).

TABLE 20
Antibodies against Entamoeba Histolytica

| Antibody No./ | | | SEQ |
|---|---|---|---|
| Antibody Name | Description | Reference Information | ID NO |
| ENTH1 | Heavy chain (partial sequence) gamma, | Cheng, X. J. et al., Exp. Parasitol. 96 | 22664 |
| | Entamoeba histolytica antibody | (1), 52-56 (2000), NCBI Accession # | |
| | | BAA97670.1 (220aa) | |
| ENTH2 | Heavy chain (partial sequence) gamma, | Tachibana, H. et al., Clin. Diagn. Lab. | 22665 |
| | Entamoeba histolytica Antibody | Immunol. 6 (3), 383-387 (1999), NCBI | |
| | | Accession # BAA82104.1 (222aa) | |
| ENTH3 | Heavy chain (partial sequence) gamma, | Tachibana, H. et al., Clin. Diagn. Lab. | 22666 |
| | Entamoeba histolytica Antibody | Immunol. 6 (3), 383-387 (1999), NCBI | |
| | | Accession # BAA82101.1 (226aa) | |
| ENTH4 | Heavy chain (partial sequence) IgG, | Tachibana, H., et al., Infect. Immun. 77 | 22667 |
| | Entamoeba histolytica Intermediate | (1), 549-556 (2009), NCBI Accession # | |
| | Subunit Lectin-Specific Human | BAH03695.1 (220aa) | |
| | Monoclonal Antibodies | | |
| ENTH5 | Heavy chain (partial sequence) IgG, | Tachibana, H., el al., Infect. Immun. 77 | 22668 |
| | Entamoeba histolytica Intermediate | (1), 549-556 (2009), NCBI Accession # | |
| | Subunit Lectin-Specific Human | BAH03694.1 (226aa) | |
| | Monoclonal Antibodies | | |
| ENTH6 | Heavy chain (partial sequence) IgG, | Tachibana, H., et al., Infect. Immun. 77 | 22669 |
| | Entamoeba histolytica Intermediate | (1), 549-556(2009), NCBI Accession # | |
| | Subunit Lectin-Specific Human | BAH03693.1 (221aa) | |
| | Monoclonal Antibodies | | |
| ENTH7 | Heavy chain (partial sequence) IgG, | Tachibana. H., et al., Infect. Immun. 77 | 22670 |
| | Entamoeba histolytica Intermediate | (1), 549-556 (2009), NCBI Accession # | |
| | Subunit Lectin-Specific Human | BAH03692.1 (223aa) | |
| | Monoclonal Anybodies | | |
| ENTH8 | Light chain (partial sequence) IgG, | Tachibana, H., et al., Infect. Immun. 77 | 22671 |
| | Entamoeba histolytica intermediate | (1), 549-556 (2009), NCBI Accession # | |
| | Subunit Lectin-Specific Human | BAH03699.1 (219aa) | |
| | Monoclonal Antibodies | | |
| ENTH9 | Light chain (partial sequence) IgG, | Tachibana. H., et al., Infect. Immun. 77 | 22672 |
| | Entamoeba histolytica Intermediate | (1), 549-556 (2009), NCBI Accession # | |
| | Subunit Lectin-Specific Human | BAH03698.1 (220aa) | |
| | Monoclonal Antibodies | | |
| ENTH10 | Light chain (partial sequence) IgG, | Tachibana, H., et al., Infect. Immun. 77 | 22673 |
| | Entamoeba histolytica Intermediate | (1), 549-556 (2009), NCBI Accession # | |
| | Subunit Lectin-Specific Human | BAH03697.1 (214aa) | |
| | Monoclonal Antibodies | | |
| ENTH11 | Light chain (partial sequence) IgG, | Tachibana, H., et al., Infect. Immun. 77 | 22674 |
| | Entamoeba histolytica Intermediate | (1), 549-556 (2009), NCBI Accession # | |
| | Subunit Lectin-Specific Human | BAH03696.1 (214aa) | |
| | Monoclonal Antibodies | | |
| ENTH12 | Light chain (partial sequence) kappa, | Cheng, X. J. et al., Exp. Parasitol. 96 (1), | 22675 |
| | Entamoeba histolytica antibody | 52-56 (2000), NCBI Accession # | |
| | | BAA97671.1 (214aa) | |
| ENTH13 | Light chain (partial sequence) kappa, | Tachibana, H. et al., Clin. Diagn. Lab. | 22676 |
| | Entamoeba histolytica antibody | Immunol. 6 (3), 383-387 (1999), NCBI | |
| | | Accession # BAA821051 (215aa) | |
| ENTH14 | Light chain (partial sequence) kappa, | Tachibana, H. et al., Clin. Diagn. Lab. | 22677 |
| | Entamoeba histolytica antibody | Immunol. 6 (3), 383-387 (1999), NCBI | |
| | | Accession # BAA82100.1 (214aa) | |
| ENTH15/ | Single chain Fv Antibody 350-E2 | NCBI Accession # AEY80059.1 (274aa) | 22678 |
| 350-E2 | against Entamoeba histolytica | | |
| ENTH16/ | Single chain Fv Antibody JR4A11 | NCBI Accession # AEY80058.1 (287aa) | 22679 |
| JR4A11 | Entamoeba histolytica | | |

In some embodiments, the payload region of the viral particle comprises one or more nucleic acid sequences encoding one or more of the payload antibody polypeptides, fragments or variants thereof described in International Pub. No. WO2001012646, the contents of which are herein incorporated by reference in their entirety, against Listeria monocytogenes, salmonella and/or leishmania.

### Neglected Tropical Diseases

In some embodiments, the payload region of the viral particle comprises one or more nucleic acid sequences encoding one or more of the neglected tropical disease related payload antibody polypeptides listed in Tables 21-24.

In some embodiments, the payload region of the viral particle comprises one or more nucleic acid sequences encoding one or more of the payload antibody polypeptides listed in Table 21 against Dengue Fever Virus (DENG1-DENG123; SEQ ID NO: 22680422802).

TABLE 21
Antibodies against Dengue Fever Virus

| Antibody | | Antibody | | SEQ |
|---|---|---|---|---|
| No. | Description | Name | Reference Information | ID NO |
| DENG1 | Bispecific, DENV serotype 1, | m3666 | US20150218255 SEQ ID NO: 96 | 22680 |
| | DENV serotype 2, DENV | | | |
| | serotype 3, and DENV serotype 4 | | | |
| DENG2 | Fab Fragment | Fab 14c10 | Teoh, E. P., el al., Sci Transl Med 4 | 22681 |
| | | | (139), 139RA83 (2012), NCBI | |
| | | | Accession # 4CAU_E(230 aa) | |
| DENG3 | Heavy chain | 5j7 Fab | Fibriansah, G., el al., A highly | 22682 |
| | | | potent human antibody neutralizes | |
| | | | dengue virus serotype 3 by binding | |
| | | | across three surface proteins; Nat | |
| | | | Commun 6, 6341 (2015), NCBI | |
| | | | Accession # 3J6U_H (135aa) | |
| DENG4 | Heavy Chain | Ede1 C8 | Dejnirattisai, W., et al., A new | 22683 |
| | | | class of highly potent, broadly | |
| | | | neutralizing antibodies isolated | |
| | | | from viremic patients infected with | |
| | | | dengue virus; Nat. Immunol. 16 | |
| | | | (2), 170-177 (2015), NCBI | |
| | | | Accession # 4UTA_H (272 aa) | |
| DENG5 | Heavy Chain | Fab 2h12 | Midgley, C. M., et al., J, Immunol. | 22684 |
| | | | 188 (10), 4971-4979 (2012), NCBI | |
| | | | Accession # 4AL8_H (217 aa) | |
| DENG6 | Heavy Chain Fab Fragment Of | 1f4 Fab | Fibriansah, G., et al., A potent anti- | 22685 |
| | Antibodv 1f4 | | dengue human antibody | |
| | | | preferentially recognizes the | |
| | | | conformation of E protein | |
| | | | monomers assembled on the virus | |
| | | | surface; EMBO Mol Med 6 (3), | |
| | | | 358-371 (2014), NCBI Accession | |
| | | | # 4C2I_H (232 aa) | |
| DENG7 | Heavy chain variable region | 9Fl 2 | WO2010093335 SEQ ID NO: 4 | 22686 |
| DENG8 | Heavy chain variable region, | 9F12 | US20150218255 SEQ ID NO: 83 | 22687 |
| | DENV serotype 1, DENV | | | |
| | serotype 2, DENV serotype 3, and | | | |
| | DENV serotype 4 | | | |
| DENG9 | Heavy chain variable region, | m366 | US20150218255 SEQ ID NO: 4 | 22688 |
| | DENV serotype 1, DENV | | | |
| | serotype 2, DENV serotype 3, and | | | |
| | DENV serotype 4 | | | |
| DENG10 | Heavy chain variable region, | m366.6 | US20150218255 SEQ ID NO: 24 | 22689 |
| | DENV serotype 1, DENV | | | |
| | serotype 2, DENV serotype 3, and | | | |
| | DENV serotype 4 | | | |
| DENG11 | Heavy chain variable region, | m360.6 | US20150218255 SEQ ID NO: 44 | 22690 |
| | DENV serotype 1, DENV | | | |
| | serotype 2, DENV serotype 3, and | | | |
| | DENV serotype 4 | | | |
| DENG12 | Heavy chain variable region, | HMB-DV-1 | U.S. Pat. No. 9,073,981 SEQ ID NO: 13 | 22691 |
| | DENV-I, DENV-2, DENV-3, | | | |
| | DENV-4 | | | |
| DENG13 | Heavy chain variable region, | HMB-DV-2 | U.S. Pat. No. 9,073,981 SEQ ID NO: 29 | 22692 |
| | DENV-I, DENV-2, DENV-3, | | | |
| | DENV-4 | | | |
| DENG14 | Heavy chain variable region, | HMB-DV-3 | U.S. Pat. No. 9,073,981 SEQ ID NO: 45 | 22693 |
| | DENV-I, DENV-2, DENV-3, | | | |
| | DENV-4 | | | |
| DENG15 | Heavy chain variable region, | HMB-DV-4 | U.S. Pat. No. 9,073,981 SEQ ID NO: 61 | 22694 |
| | DENV-I, DENV-2, DENV-3, | | | |
| | DENV-4 | | | |
| DENG16 | Heavy chain variable region, | HMB-DV-4 | U.S. Pat. No. 9,073,981 SEQ ID NO: 65 | 22695 |
| | DENV-I, DENV-2, DENV-3, | | | |
| | DENV-4 | | | |
| DENG17 | Heavy chain variable region, | HMB-DV-5 | U.S. Pat. No. 9,073,981 SEQ ID NO: 79 | 22696 |
| | DENV-I, DENV-2, DEN V-3, | | | |
| | DENV-4 | | | |
| DENG18 | Heavy chain variable region, | HMB-DV-6, | U.S. Pat. No. 9,073,981 SEQ ID NO: 95 | 22697 |
| | DENV-I, DENV-2, DENV-3, | HMB-DV-7 | | |
| | DENV-4 | | | |
| DENG19 | Heavy chain variable region, | HMB-DV-8 | U.S. Pat. No. 9,073,981 SEQ ID NO: 117 | 22698 |
| | DENV-I, DENV-2, DENV-3, | | | |
| | DENV-4 | | | |
| DENG20 | Heavy chain variable region, | HMB-DV-9 | U.S. Pat. No. 9,073,981 SEQ ID NO: 131 | 22699 |
| | DENV-I, DENV-2, DENV-3, | | | |
| | DENV-4 | | | |
| DENG21 | Heavy chain variable region, | HMB-DV-10 | U.S. Pat. No. 9,073,981 SEQ ID NO: 145 | 22700 |
| | DENV-I, DENV-2, DENV-3, | | | |
| | DENV-4 | | | |
| DENG22 | Heavy chain variable region, | HMB-DV-11 | U.S. Pat. No. 9,073,981 SEQ ID NO: 151 | 22701 |
| | DENV-I, DENV-2, DENV-3, | | | |
| | DENV-4 | | | |
| DENG23 | Heavy chain variable region, | HMB-DV-12 | U.S. Pat. No. 9,073,981 SEQ ID NO: 165 | 22702 |
| | DENV-I, DENV-2, DENV-3, | | | |
| | DENV-4 | | | |
| DENG24 | Heavy chain variable region, | HMB-DV-13 | U.S. Pat. No. 9,073,981 SEQ ID NO: 181 | 22703 |
| | DENV-I, DENV-2, DENV-3, | | | |
| | DENV-4 | | | |
| DENG25 | Heavy chain variable region, | HMB-DV-14 | U.S. Pat. No. 9,073,981 SEQ ID NO: 195 | 22704 |
| | DENV-I, DENV-2, DENV-3, | | | |
| | DENV-4 | | | |
| DENG26 | Heavy chain variable region, DV- | A68 | US20150225474 SEQ ID NO: 19 | 22705 |
| | 1, DV-2, DV-3, and DV-10 | | | |
| DENG27 | Heavy chain variable region, DV- | A100 | US20150225474 SEQ ID NO: 20 | 22706 |
| | 1, DV-2, DV-3, and DV-11 | | | |
| DENG28 | Heavy chain variable region, DV- | C58 | US20150225474 SEQ ID NO: 21 | 22707 |
| | 1, DV-2, DV-3, and DV-12 | | | |
| DENG29 | Heavy chain variable region, DV- | C98 | US20150225474 SEQ ID NO: 32 | 22708 |
| | 1, DV-2, DV-3, and DV-13 | | | |
| DENG30 | Heavy chain variable region, DV- | A11 | US20150225474 SEQ ID NO: 33 | 22709 |
| | 1, DV-2, DV-3, and DV-14 | | | |
| DENG31 | Heavy chain variable region, DV- | B11 | US20150225474 SEQ ID NO: 36 | 22710 |
| | 1, DV-2, DV-3, and DV-15 | | | |
| DENG32 | Heavy chain variable region, DV- | D88 | US20150225474 SEQ ID NO: 1 | 22711 |
| | 1, DV-2, DV-3, and DV-4 | | | |
| DENG33 | Heavy chain variable region, DV- | mAb11 | WO2014144061 SEQ ID NO: 1 | 22712 |
| | 1, DV-2, DV-3, and DV-1 | | | |
| DENG34 | Heavy chain variable region, DV- | F38 | US20150225474 SEQ ID NO: 80 | 22713 |
| | 1, DV-2, DV-3, and DV-5 | | | |
| DENG35 | Heavy chain variable region, DV- | A48 | US20150225474 SEQ ID NO: 16 | 22714 |
| | 1, DV-2, DV-3, and DV-6 | | | |
| DENG36 | Heavy drain variable region, DV- | C88 | US20150225474 SEQ ID NO: 17 | 22715 |
| | 1, DV-2, DV-3, and DV-7 | | | |
| DENG37 | Heavy chain variable region, DV- | F108 | US20150225474 SEQ ID NO: 18 | 22716 |
| | 1, DV-2, DV-3, and DV-8 | | | |
| DENG38 | Heavy chain variable region, DV- | B48 | US20150225474 SEQ ID NO: 18 | 22717 |
| | 1, DV-2, DV-3, and DV-9 | | | |
| DENG39 | Heavy chain, Antigen-binding | 2d22 | Fibriansah, G., et al., DENGUE | 22718 |
| | Fragment Of Human Antibody | | VIRUS. Cryo-EM structure of an | |
| | 2d22 | | antibody that neutralizes dengue | |
| | | | virus type 2 by locking E protein | |
| | | | dimers; Science 349 (6243), 88-91 | |
| | | | (2015), NCBI Accession # | |
| | | | 5A1Z_K (128 aa) | |
| DENG40 | Heavy chain, Dengue virus NS-1 | | U.S. Pat. No. 7,473,424; US20040209244; | 22719 |
| | protein | | WO2004067567; EP1592712 SEQ ID NO: 3 | |
| DENG41 | Heavy chain, Dengue virus | DB32-6 | U.S. Pat. No. 8,637,035 SEQ ID NO: 1 | 22720 |
| | serotype 2 | | | |
| DENG42 | Heavy chain, Dengue virus | DB2-3 | U.S. Pat. No. 8,637,035 SEQ ID NO: 13 | 22721 |
| | serotype 2 | | | |
| DENG43 | Heavy chain, Dengue virus | DB13-19 | U.S. Pat. No. 8,637,035 SEQ ID NO: 14 | 22722 |
| | serotype 2 | | | |
| DENG44 | Heavy chain, Dengue virus | DB23-3 | U.S. Pat. No. 8,637,035 SEQ ID NO: 15 | 22723 |
| | serotype 2 | | | |
| DENG45 | Heavy chain, Dengue virus | DB25-2 | U.S. Pat. No. 8,637,035 SEQ ID NO: 16 | 22724 |
| | serotype 2 | | | |
| DENG46 | Heavy chain, Dengue virus | DB42-3 | U.S. Pat. No. 8,637,035 SEQ ID NO: 17 | 22725 |
| | serotype 2 | | | |
| DENG47 | Heavy chain, Dengue virus type | 1A5 | U.S. Pat. No. 8,337,853 SEQ ID NO: 97 | 22726 |
| | 10 | | | |
| DENG48 | Heavy chain, Dengue virus type | 2H7 | U.S. Pat. No. 8,337,853 SEQ ID NO: 113 | 22727 |
| | 11 | | | |
| DENG49 | Heavy chain, Dengue virus type | 2H5 | U.S. Pat. No. 8,337,853 SEQ ID NO: 129 | 22728 |
| | 12 | | | |
| DENG50 | Heavy chain, Dengue virus type | 3A2 | US20130089543 SEQ ID NO: 145 | 22729 |
| | 13 | | | |
| DENG51 | Heavy chain, Dengue virus type | 1B2 | US20130089543 SEQ ID NO: 161 | 22730 |
| | 14 | | | |
| DENG52 | Heavy chain, Dengue virus type | 1A10 | US20130089543 SEQ ID NO: 177 | 22731 |
| | 15 | | | |
| DENG53 | Heavy chain, Dengue virus type 4 | 5H2 | U.S. Pat. No. 7,622,113 SEQ ID NO: 1 | 22732 |
| DENG54 | Heavy chain, Dengue virus type 5 | 5A7 | U.S. Pat. No. 7,622,113 SEQ ID NO: 17 | 22733 |
| DENG55 | Heavy chain, Dengue vires type 6 | 3C1 | U.S. Pat. No. 7,622,113 SEQ ID NO: 33 | 22734 |
| DENG56 | Heavy chain, Dengue virus type 7 | 3E4 | U.S. Pat. No. 7,622,113 SEQ ID NO: 49 | 22735 |
| DENG57 | Heavy chain, Dengue virus type 8 | 7G4 | U.S. Pat. No. 7,622,113 SEQ ID NO: 65 | 22736 |
| DENG58 | Heavy chain, Dengue virus type 9 | 5D9 | U.S. Pat. No. 7,622,113 SEQ ID NO: 81 | 22737 |
| DENG59 | Heavy chain, DV 1 | 14c10 clone | US20130259871 FIG. 4b | 22738 |
| | | 8 | | |
| DENG60 | Heavy chain, DV-1, DV-2, DV-3, | Antibody | US20140056913 SEQ ID NO: 1 | 22739 |
| | and DV-4 | 4e11 | | |
| DENG61 | Heavy chain, DV-1, DV-2, DV-3, | Variant of | US20140056913 SEQ ID NO: 21 | 22740 |
| | and DV-4 | 4E11 | | |
| DENG62 | Heavy chain, DV-1, DV-2, DV-3, | 4E5A | WO20155123362 SEQ ID NO: 29 | 22741 |
| | and DV-4 | | | |
| DENG63 | Light Chain | 5j7 Fab | Fibriansah, G., et al., A highly | 22742 |
| | | | potent human antibody neutralizes | |
| | | | dengue virus serotype 3 by binding | |
| | | | across three surface proteins; Nat | |
| | | | Commun 6, 6341 (2015), NCBI | |
| | | | Accession # 3J6U_L (118aa) | |
| DENG64 | Light Chain | Ede1 C8 | Dejnirattisai, W., et al., A new | 22743 |
| | | | class of highly potent, broadly | |
| | | | neutralizing antibodies isolated | |
| | | | from viremic patients infected with | |
| | | | dengue virus; Nat. Immunol. 16 | |
| | | | (2), 170-177 (2015), NCBI | |
| | | | Accession # 4UTA_L (217 aa) | |
| DENG65 | Light Chain | Fab 2h12 | Midgley, C. M., et al., J, Immunol. | 22744 |
| | | | 188 (10), 4971-4979 (2012), NCBI | |
| | | | Accession # 4AL8_L (213 aa) | |
| DENG66 | Light Chain Fab Fragment Of | 1f4 Fab | Fibriansah, G., et al., A potent anti- | 22745 |
| | Antibody 1f4 | | dengue human antibody | |
| | | | preferentially recognizes the | |
| | | | conformation of E protein | |
| | | | monomers assembled on the virus | |
| | | | surface; EMBO Mol Med 6 (3), | |
| | | | 358-371 (2014), NCBI Accession | |
| | | | # 4C2I_N (239 aa) | |
| DENG67 | Light chain variable region | 9Fl 2 | WO2010093335 SEQ ID NO: 6 | 22746 |
| DENG68 | Light chain variable region, | 9F12 | US20150218255 SEQ ID NO: 84 | 22747 |
| | DENV serotype 1, DENV | | | |
| | serotype 2, DENV serotype 3, and | | | |
| | DENV serotype 4 | | | |
| DENG69 | Light chain variable region, | m366 | US20150218255 SEQ ID NO: 6 | 22748 |
| | DENV serotype 1, DENV | | | |
| | serotype 2, DENV serotype 3, and | | | |
| | DENV serotype 4 | | | |
| DENG70 | Light chain variable region, | m366.6 | US20150218255 SEQ ID NO: 26 | 22749 |
| | DENV serotype 1, DENV | | | |
| | serotype 2, DENV serotype 3, and | | | |
| | DENV serotype 4 | | | |
| DENG71 | Light chain variable region, | m360.6 | US20150218255 SEQ ID NO: 46 | 22750 |
| | DENV serotype 1, DENV | | | |
| | serotype 2, DENV serotype 3, and | | | |
| | DENV serotype 4 | | | |
| DENG72 | Light chain variable region, | HMB-DV-1 | U.S. Pat. No. 9,073,981 SEQ ID NO: 14 | 22751 |
| | DENV-I, DENV-2, DENV-3, | | | |
| | DENV-4 | | | |
| DENG73 | Light chain variable region, | HMB-DV-2 | U.S. Pat. No. 9,073,981 SEQ ID NO: 30 | 22752 |
| | DENV-I, DENV-2, DENV-3, | | | |
| | DENV-4 | | | |
| DENG74 | Light chain variable region, | HMB-DV-3 | U.S. Pat. No. 9,073,981 SEQ ID NO: 46 | 22753 |
| | DENV-I, DENV-2, DENV-3, | | | |
| | DENV-4 | | | |
| DENG75 | Light chain variable region, | HMB-DV-4 | U.S. Pat. No. 9,073,981 SEQ ID NO: 62 | 22754 |
| | DENV-I, DENV-2, DENV-3, | | | |
| | DENV-4 | | | |
| DENG76 | Light chain variable region, | HMB-DV-5 | U.S. Pat. No. 9,073,981 SEQ ID NO: 80 | 22755 |
| | DENV-I, DENV-2, DENV-3, | | | |
| | DENV-4 | | | |
| DENG77 | Light chain variable region, | HMB-DV-6 | U.S. Pat. No. 9,073,981 SEQ ID NO: 96 | 22756 |
| | DENV-I, DENV-2, DENV-3, | | | |
| | DENV-4 | | | |
| DENG78 | Light chain variable region, | HMB-DV-7 | U.S. Pat. No. 9,073,981 SEQ ID NO: 103 | 22757 |
| | DENV-I, DENV-2, DENV-3, | | | |
| | DENV-4 | | | |
| DENG79 | Light chain variable region, | HMB-DV-8 | U.S. Pat. No. 9,073,981 SEQ ID NO: 118 | 22758 |
| | DENV-I, DENV-2, DENV-3, | | | |
| | DENV-4 | | | |
| DENG80 | Light chain variable region, | HMB-DV-9 | U.S. Pat. No. 9,073,981 SEQ ID NO: 132 | 22759 |
| | DENV-I, DENV-2, DENV-3, | | | |
| | DENV-4 | | | |
| DENG81 | Light chain variable region, | HMB-DV-10, | U.S. Pat. No. 9,073,981 SEQ ID NO: 146 | 22760 |
| | DENV-I, DENV-2, DENV-3, | HMB-DV-11 | | |
| | DENV-4 | | | |
| DENG82 | Light chain variable region, | HMB-DV-12 | U.S. Pat. No. 9,073,981 SEQ ID NO: 166 | 22761 |
| | DENV-I, DENV-2, DENV-3, | | | |
| | DENV-4 | | | |
| DENG83 | Light chain variable region, | HMB-DV-13 | U.S. Pat. No. 9,073,981 SEQ ID NO: 182 | 22762 |
| | DENV-I, DENV-2, DENV-3, | | | |
| | DENV-4 | | | |
| DENG84 | Light chain variable region, | HMB-DV-14 | U.S. Pat. No. 9,073,981 SEQ ID NO: 196 | 22763 |
| | DENV-I, DENV-2, DENV-3, | | | |
| | DENV-4 | | | |
| DENG85 | Light chain variable region, DV-1, | D88, F38, | US20150225474 SEQ ID NO: 2 | 22764 |
| | DV-2, DV-3, and DV-4 | A48, C88, | | |
| | | F108, B48, | | |
| | | A68, A100, | | |
| | | C58, C78, | | |
| | | C68, D98, | | |
| | | D188, C128, | | |
| | | C98 | | |
| DENG86 | Light chain variable region, DV-1, | C78 | US20S50225474 SEQ ID NO: 23 | 22765 |
| | DV-2, DV-3, and DV-4 | | | |
| DENG87 | Light chain variable region, DV-1, | C68 | US20150225474 SEQ ID NO: 25 | 22766 |
| | DV-2, DV-3, and DV-4 | | | |
| DENG88 | Light chain variable region, DV-1, | D98 | US20150225474 SEQ ID NO: 27 | 22767 |
| | DV-2, DV-3, and DV-4 | | | |
| DENG89 | Light chain variable region, DV-1, | D188 | US20150225474 SEQ ID NO: 29 | 22768 |
| | DV-2, DV-3, and DV-4 | | | |
| DENG90 | Light chain variable region, DV-1, | C128 | US20150225474 SEQ ID NO: 31 | 22769 |
| | DV-2, DV-3, and DV-4 | | | |
| DENG91 | Light chain variable region, DV-1, | A11, B11 | US20150225474 SEQ ID NO: 34 | 22770 |
| | DV-2, DV-3, and DV-4 | | | |
| DENG92 | Light chain variable region, DV-1, | mAb11 | WO2014144061 SEQ ID NO: 3 | 22771 |
| | DV-2, DV-3, and DV-4 | | | |
| DENG93 | Light chain, Antigen-binding | 2d22 | Fibriansah, G., el al., DENGUE | 22772 |
| | Fragment Of Human Antibody | | VIRUS. Cryo-EM structure of an | |
| | 2d22 | | antibody that neutralizes dengue | |
| | | | virus type 2 by locking E protein | |
| | | | dimers; Science 349 (6243), 88-91 | |
| | | | (2015), NCBI Accession # | |
| | | | 5A1Z_L (115 aa) | |
| DENG94 | Light chain, Dengue virus NS-1 | | U.S. Pat. No. 7,473,424; US20040209244; | 22773 |
| | protein | | WO2004067567; EP1592712 SEQ ID NO: 4 | |
| DENG95 | Light chain, Dengue virus | DB32-6 | U.S. Pat. No. 8,637,035 SEQ ID NO: 5 | 22774 |
| | serotype 2 | | | |
| DENG96 | Light chain, Dengue virus | DB2-3, | U.S. Pat. No. 8,637,035 SEQ ID NO: 19 | 22775 |
| | serotype 2 | DB-19 | | |
| DENG97 | Light chain, Dengue virus | DB23-3 | U.S. Pat. No. 8,637,035 SEQ ID NO: 20 | 22776 |
| | serotype 2 | | | |
| DENG98 | Light chain, Dengue virus | DB25-2 | U.S. Pat. No. 8,637,035 SEQ ID NO: 21 | 22777 |
| | serotype 2 | | | |
| DENG99 | Light chain, Dengue virus | DB42-3 | U.S. Pat. No. 8,637,035 SEQ ID NO: 22 | 22778 |
| | serotype 2 | | | |
| DENG100 | Light chain, Dengue virus | 5H2 | U.S. Pat. No. 7,622,113 SEQ ID NO: 9 | 22779 |
| | serotype 4 | | | |
| DENG101 | Light chain, Dengue virus | 5A7 | U.S. Pat. No. 7,622,113 SEQ ID NO: 25 | 22780 |
| | serotype 4 | | | |
| DENG102 | Light chain, Dengue virus | 3C1 | U.S. Pat. No. 7,622,113 SEQ ID NO: 41 | 22781 |
| | serotype 4 | | | |
| DENG103 | Light chain, Dengue virus | 3E4 | U.S. Pat. No. 7,622,113 SEQ ID NO: 57 | 22782 |
| | serotype 4 | | | |
| DENG104 | Light chain, Dengue virus | 7G4 | U.S. Pat. No. 7,622,113 SEQ ID NO: 73 | 22783 |
| | serotype 4 | | | |
| DENG105 | Light chain, Dengue virus | 5D9 | U.S. Pat. No. 7,622,153 SEQ ID NO: 89 | 22784 |
| | serotype 4 | | | |
| DENG106 | Light chain, Dengue virus | 1A5 | U.S. Pat. No. 8,337,853 SEQ ID NO: 105 | 22785 |
| | serotype 4 | | | |
| DENG107 | Light chain, Dengue virus | 2H7 | U.S. Pat. No. 8,337,853 SEQ ID NO: 121 | 22786 |
| | serotype 4 | | | |
| DENG108 | Light chain, Dengue virus | 2H5 | U.S. Pat. No. 8,337,853 SEQ ID NO: 137 | 22787 |
| | serotype 4 | | | |
| DENG109 | Light chain, Dengue virus | 3A2 | US20130089543 SEQ ID NO: 153 | 22788 |
| | serotype 4 | | | |
| DENG110 | Light chain, Dengue virus | 1B2 | US20130089543 SEQ ID NO: 169 | 22789 |
| | serotype 4 | | | |
| DENG111 | Light chain, Dengue virus | 1A10 | US20130089543 SEQ ID NO: 185 | 22790 |
| | serotype 4 | | | |
| DENG112 | Light chain, DV 1 | 14c10 clone | US20130259871 FIG. 4b | 22791 |
| | | 8 | | |
| DENG113 | Light chain, DV-1, DV-2, DV-3, | Antibody | US20140056913 SEQ ID NO: 2 | 22792 |
| | and DV-4 | 4e11 | | |
| DENG114 | Light chain, DV-1, DV-2, DV-3, | Variant of | US20140056913 SEQ ID NO: 22 | 22793 |
| | and DV-4 | 4E11 | | |
| DENG115 | Light chain, DV-l, DV-2, DV-3, | 4E5A | WO20155123362 SEQ ID NO: 30 | 22794 |
| | and DV-4 | | | |
| DENG116 | scFv | 9Fl 2 | WO2010093335 SEQ ID NO: 8 | 22795 |
| DENG117 | Scfv Fragment | Ede2 A11 | Dejnirattisai, W., et al., A new | 22796 |
| | | | class of highly potent, broadly | |
| | | | neutralizing antibodies isolated | |
| | | | from viremic patients infected with | |
| | | | dengue virus; Nat. Immunol. 16 | |
| | | | (2), 170-177 (2015), NCBI | |
| | | | Accession # 4UT7_L(153 aa) | |
| DENG118 | Scfv Fragment | Ede2 A11 | Dejnirattisai, W., et al, A new | 22797 |
| | | | class of highly potent, broadly | |
| | | | neutralizing antibodies isolated | |
| | | | from viremic patients infected with | |
| | | | dengue virus; Nat. Immunol. 16 | |
| | | | (2), 170-177 (2015), NCBI | |
| | | | Accession # 4UT7_H (150 aa) | |
| DENG119 | | Ede2 A11 | Dejnirattisai, W., et al., A new | 22798 |
| | | | class of lightly potent, broadly | |
| | | | neutralizing antibodies isolated | |
| | | | from viremic patients infected with | |
| | | | dengue virus; Nat. Immunol. 16 | |
| | | | (2), 170-177 (2015), NCBI | |
| | | | Accession # 4UTB_L (218 aa) | |
| DENG120 | | Ede1 C10 | Dejnirattisai, W., et al., A new | 22799 |
| | | | class of highly potent, broadly | |
| | | | neutralizing antibodies isolated | |
| | | | from viremic patients infected with | |
| | | | dengue virus; Nat. Immunol. 16 | |
| | | | (2), 170-177 (2015), NCBI | |
| | | | Accession # 4UT9_L (154aa) | |
| DENG121 | | Ede1 C10 | Dejnirattisai, W., et al., A new | 22800 |
| | | | class of highly potent, broadly | |
| | | | neutralizing antibodies isolated | |
| | | | from viremic patients infected with | |
| | | | dengue virus; Nat. Immunol. 16 | |
| | | | (2), 170-177 (2015), NCBI | |
| | | | Accession # 4UT9_H (144 aa) | |
| DENG122 | | Ede2 B7 | Dejnirattisai, W., et al., A new | 22801 |
| | | | class of highly potent, broadly | |
| | | | neutralizing antibodies isolated | |
| | | | from viremic patients infected with | |
| | | | dengue virus; Nat. Immunol. 16 | |
| | | | (2), 170-177 (2015), NCBI | |
| | | | Accession # 4UT6_L (218 aa) | |
| DENG123 | | Ede2 B7 | Dejnirattisai, W., et al., A new | 22802 |
| | | | class of highly potent, broadly | |
| | | | neutralizing antibodies isolated | |
| | | | from viremic patients injected with | |
| | | | dengue virus; Nat. Immunol. 16 | |
| | | | (2), 170-177 (2015), NCBI | |
| | | | Accession # 4UT6_H (283 aa) | |

In some embodiments, the payload region of the viral particle comprises one or more nucleic acid sequences encoding one or more of the payload antibody polypeptides, fragments or variants thereof described in International Pub. No. WO2013089647 and WO2013035345, U.S. Pat. No. 8,637,035 and US887187, US Publication No. US20050123900, and Chinese Patent Publication No, CN102757480, the contents of which are herein incorporated by reference in their entirety, against Listeria monocytogenes, salmonella and/or leishmania.

In some embodiments, the payload region of the viral particle comprises one or more nucleic acid sequences encoding one or more of the payload antibody polypeptides listed in Table 22 against Rabies Virus (RABV1-RABV91; SECS ID NO: 22803-22893).

TABLE 22
Antibodies against Rabies Virus

| Antibody | | | | SEQ |
|---|---|---|---|---|
| No. | Description | Antibody Name | Reference Information | ID NO |
| RABV1 | Fab Heavy Chain Fd region | | CN101696242 SEQ ID NO: 9 | 22803 |
| RABV2 | Fab Light chain | | CN101696242 SEQ ID NO: 10 | 22804 |
| RABV3 | Heavy chain | | U.S. Pat. No. 6,890,532 SEQ ID NO: 3 | 22805 |
| RABV4 | Heavy chain | Mab JB.1 | U.S. Pat. No. 7,071,319 SEQ ID NO: 10 | 22806 |
| RABV5 | Heavy chain | Mab 57 | U.S. Pat. No. 7,071,319 SEQ ID NO: 14 | 22807 |
| RABV6 | Heavy chain | CR04-098 | U.S. Pat. No. 9,005,624 SEQ ID NO: 335 | 22808 |
| RABV7 | Heavy chain | CR57, | U.S. Pat. No. 9,005,624 SEQ ID NO: 123 | 22809 |
| | | Rafivirumab | | |
| RABV8 | Heavy chain | CR57, | | 22810 |
| | | Rafivirumab | | |
| RABV9 | Heavy chain | CRJB | U.S. Pat. No. 9,005,624 SEQ ID NO: 127 | 22811 |
| RABV10 | Heavy chain | Foravirumab | | 22812 |
| RABV11 | Heavy chain, Anti-rabies SOJB | | Presniak, M. et al. | 22813 |
| | immunoglobulin | | “Development of a cocktail of | |
| | | | recombinant-expressed human | |
| | | | rabies virus-neutralizing | |
| | | | monoclonal antibodies for | |
| | | | postexposure prophylaxis of | |
| | | | rabies”, J. Infect. Dis. 188 (1), | |
| | | | 53-56 (2003), NCBI | |
| | | | Accession # AAO17822.1 | |
| RABV12 | Heavy chain variable region | | CN101696242 SEQ ID NO: 4 | 22814 |
| RABV13 | Heavy chain variable region | SC04-001 | U.S. Pat. No. 9,005,624 SEQ ID NO: 26 | 22815 |
| RABV14 | Heavy chain variable region | SC04-004 | U.S. Pat. No. 9,005,624 SEQ ID NO: 27 | 22816 |
| RABV15 | Heavy chain variable region | SC04-008 | U.S. Pat. No. 9,005,624 SEQ ID NO: 28 | 22817 |
| RABV16 | Heavy chain variable region | SC04-010 | U.S. Pat. No. 9,003,624 SEQ ID NO: 29 | 22818 |
| RABV17 | Heavy chain variable region | SC04-018 | U.S. Pat. No. 9,005,624 SEQ ID NO: 30 | 22819 |
| RABV18 | Heavy chain variable region | SC04-021 | U.S. Pat. No. 9,005,624 SEQ ID NO: 31 | 22820 |
| RABV19 | Heavy chain variable region | SC04-026 | U.S. Pat. No. 9,005,624 SEQ ID NO: 32 | 22821 |
| RABV20 | Heavy chain variable region | SC04-031 | U.S. Pat. No. 9,005,624 SEQ ID NO: 33 | 22822 |
| RABV21 | Heavy chain variable region | SC04-038 | U.S. Pat. No. 9,005,624 SEQ ID NO: 44 | 22823 |
| RABV22 | Heavy chain variable region | SC04-040 | U.S. Pat. No. 9,005,624 SEQ ID NO: 35 | 22824 |
| RABV23 | Heavy chain variable region | SC04-060 | U.S. Pat. No. 9,005,624 SEQ ID NO: 36 | 22825 |
| RABV24 | Heavy chain variable region | SC04-073 | U.S. Pat. No. 9,005,624 SEQ ID NO: 37 | 22826 |
| RABV25 | Heavy chain variable region | SC04-097 | U.S. Pat. No. 9,005,624 SEQ ID NO: 38 | 22827 |
| RABV26 | Heavy chain variable region | SC04-098 | U.S. Pat. No. 9,005,624 SEQ ID NO: 39 | 22828 |
| RABV27 | Heavy chain variable region | SC04-103 | U.S. Pat. No. 9,005,624 SEQ ID NO: 40 | 22829 |
| RABV28 | Heavy chain variable region | SC04-104 | U.S. Pat. No. 9,005,624 SEQ ID NO: 41 | 22830 |
| RABV29 | Heavy chain variable region | SC04-108 | U.S. Pat. No. 9,005,624 SEQ ID NO: 42 | 22831 |
| RABV30 | Heavy chain variable region | SC04-120 | U.S. Pat. No. 9,005,624 SEQ ID NO: 43 | 22832 |
| RABV31 | Heavy chain variable region | SC04-125 | U.S. Pat. No. 9,005,624 SEQ ID NO: 44 | 22833 |
| RABV32 | Heavy chain variable region | SC04-126 | U.S. Pat. No. 9,005,624 SEQ ID NO: 45 | 22834 |
| RABV33 | Heavy chain variable region | SC04-140 | U.S. Pat. No. 9,005,624 SEQ ID NO: 46 | 22835 |
| RABV34 | Heavy chain variable region | SC04-144 | U.S. Pat. No. 9,005,624 SEQ ID NO: 47 | 22836 |
| RABV35 | Heavy chain variable region | SC04-146 | U.S. Pat. No. 9,005,624 SEQ ID NO: 48 | 22837 |
| RABV36 | Heavy chain variable region | SC04-164 | U.S. Pat. No. 9,005,624 SEQ ID NO: 49 | 22838 |
| RABV37 | Heavy chain variable region | RVFab5 | WO201113757 SEQ ID NO: 2 | 22839 |
| RABV38 | Heavy chain variable region | RVFab8 | WO2011137570 SEQ ID NO: 2 | 22840 |
| RABV39 | Heavy chain variable region | | CN101337990 SEQ ID NO: 2 | 22841 |
| RABV40 | Heavy chain variable region | | CN101337990 SEQ ID NO: 8 | 22842 |
| RABV41 | Heavy chain variable region | R8 VH | CN104193823 SEQ ID NO: 1 | 22843 |
| RABV42 | Heavy chain variable region | R5 VH | CN104193823 SEQ ID NO: 2 | 22844 |
| RABV43 | Heavy chain variable region | R7 VH, R9 VH | CN104193823 SEQ ID NO: 3 | 22845 |
| RABV44 | Heavy chain variable region | | CN101235086 SEQ ID NO: 38 | 22846 |
| RABV45 | Heavy chain, Anti-rabies | | Prosniak, M. et al. | 22847 |
| | SOJA immunoglobulin | | “Development of a cocktail of | |
| | | | recombinant-expressed human | |
| | | | rabies virus-neutralizing | |
| | | | monoclonal antibodies for | |
| | | | postexposure prophylaxis of | |
| | | | rabies”, J. Infect. Dis. 188 (1), | |
| | | | 53-56 (2003), NCBI | |
| | | | Accession # AAO17823.1 | |
| RABV46 | Light chain | | U.S. Pat. No. 6,890,532 SEQ ID NO: 4 | 22848 |
| RABV47 | Light chain | Mab JB.1 | U.S. Pat. No. 7,071,319 SEQ ID NO: 12 | 22849 |
| RABV48 | Light chain | Mab 57 | U.S. Pat. No. 7,071,319 SEQ ID NO: 16 | 22850 |
| RABV49 | Light chain | CR04-098 | U.S. Pat. No. 9,005,624 SEQ ID NO: 337 | 22851 |
| RABV50 | Light chain | CR57, | U.S. Pat. No. 9,005,624 SEQ ID NO: 125 | 22852 |
| | | Rafivirumab | | |
| RABV51 | Light chain | CR57, | | 22853 |
| | | Rafivirumab | | |
| RABV52 | Light chain | CRJB | U.S. Pat. No. 9,005,624 SEQ ID NO: 129 | 22854 |
| RABV53 | Light chain | Foravirumab | | 22855 |
| RABV54 | Light chain Kappa, Anti-rabies | | Prosniak, M. et al. | 22856 |
| | SOJA immunoglobulin | | “Development of a cocktail of | |
| | | | recombinant-expressed human | |
| | | | rabies virus-neutralizing | |
| | | | monoclonal antibodies for | |
| | | | postexposure prophylaxis of | |
| | | | rabies”, J. Infect. Dis. 188 (1), | |
| | | | 53-56 (2003), NCBI | |
| | | | Accession # AAO17825.1 | |
| RABV55 | Light chain kappa, Anti-rabies | | Prosniak, M. et al. | 22857 |
| | SOJA immunoglobulin | | “Development of a cocktail of | |
| | [Homo sapiens] | | recombinant-expressed human | |
| | | | rabies virus-neutralizing | |
| | | | monoclonal antibodies for | |
| | | | postexposure prophylaxis of | |
| | | | rabies”, J. Infect. Dis. 188 (1), | |
| | | | 53-56 (2003), NCBI | |
| | | | Accession # AAO17821.1 | |
| RABV56 | Light chain Lambda, Anti-rabies | | Prosniak, M. et al. | 22858 |
| | S057 immunoglobulin | | “Development of a cocktail of | |
| | | | recombinant-expressed human | |
| | | | rabies virus-neutralizing | |
| | | | monoclonal antibodies for | |
| | | | postexposure prophylaxis of | |
| | | | rabies”, J. Infect. Dis. 188 (1), | |
| | | | 53-56 (2003), NCBI | |
| | | | Accession # AAO17824.1 | |
| RABV57 | Light chain lambda, Anti-rabies | | Prosniak, M. et al. | 22859 |
| | SOJB immunoglobulin | | “Development of a cocktail of | |
| | | | recombinant-expressed human | |
| | | | rabies virus-neutralizing | |
| | | | monoclonal antibodies for | |
| | | | postexposure prophylaxis of | |
| | | | rabies”, J. Infect. Dis. 188 (1), | |
| | | | 53-56 (2003), NCBI | |
| | | | Accession # AAO17826.1 | |
| RABV58 | Light chain variable region | SC04-001 | U.S. Pat. No. 9,005,624 SEQ ID NO: 50 | 22860 |
| RABV59 | Light chain variable region | SC04-004 | U.S. Pat. No. 9,005,624 SEQ ID NO: 51 | 22861 |
| RABV60 | Light chain variable region | SC04-008 | U.S. Pat. No. 9,005,624 SEQ ID NO: 52 | 22862 |
| RABV61 | Light chain variable region | SC04-010 | U.S. Pat. No. 9,005,624 SEQ ID NO: 55 | 22863 |
| RABV62 | Light chain variable region | SC04-018 | U.S. Pat. No. 9,005,624 SEQ ID NO: 54 | 22864 |
| RABV63 | Light chain variable region | SC04-021 | U.S. Pat. No. 9,005,624 SEQ ID NO: 55 | 22865 |
| RABV64 | Light chain variable region | SC04-026 | U.S. Pat. No. 9,005,624 SEQ ID NO: 56 | 22866 |
| RABV65 | Light chain variable region | SC04-031 | U.S. Pat. No. 9,005,624 SEQ ID NO: 57 | 22867 |
| RABV66 | Light chain variable region | SC04-038 | U.S. Pat. No. 9,005,624 SEQ ID NO: 58 | 22868 |
| RABV67 | Light chain variable region | SC04-040 | U.S. Pat. No. 9,005,624 SEQ ID NO: 59 | 22869 |
| RABV68 | Light chain variable region | SC04-060 | U.S. Pat. No. 9,005,624 SEQ ID NO: 60 | 22870 |
| RABV69 | Light chain variable region | SC04-073 | U.S. Pat. No. 9,005,624 SEQ ID NO: 61 | 22871 |
| RABV70 | Light chain variable region | SC04-097 | U.S. Pat. No. 9,005,624 SEQ ID NO: 62 | 22872 |
| RABV71 | Light chain variable region | SC04-098 | U.S. Pat. No. 9,005,624 SEQ ID NO: 63 | 22873 |
| RABV72 | Light chain variable region | SC04-103 | U.S. Pat. No. 9,005,624 SEQ ID NO: 64 | 22874 |
| RABV73 | Light chain variable region | SC04-104 | U.S. Pat. No. 9,005,624 SEQ ID NO: 65 | 22875 |
| RABV74 | Light chain variable region | SC04-108 | U.S. Pat. No. 9,005,624 SEQ ID NO: 66 | 22876 |
| RABV75 | Light chain variable region | SC04-120 | U.S. Pat. No. 9,005,624 SEQ ID NO: 67 | 22877 |
| RABV76 | Light chain variable region | SC04-125 | U.S. Pat. No. 9,005,624 SEQ ID NO: 68 | 22878 |
| RABV77 | Light chain variable region | SC04-126 | U.S. Pat. No. 9,005,624 SEQ ID NO: 69 | 22879 |
| RABV78 | Light chain variable region | SC04-140 | U.S. Pat. No. 9,005,624 SEQ ID NO: 70 | 22880 |
| RABV79 | Light chain variable region | SC04-144 | U.S. Pat. No. 9,005,624 SEQ ID NO: 71 | 22881 |
| RABV80 | Light chain variable region | SC04-146 | U.S. Pat. No. 9,005,624 SEQ ID NO: 72 | 22882 |
| RABV81 | Light chain variable region | SC04-164 | U.S. Pat. No. 9,005,624 SEQ ID NO: 73 | 22883 |
| RABV82 | Light chain variable region | RVFab5 | WO201113757 SEQ ID NO: 1 | 22884 |
| RABV83 | Light chain variable region | RVFab8 | WO2011137570 SEQ ID NO: 1 | 22885 |
| RABV84 | Light chain variable region | | CN101337990 SEQ ID NO: 4 | 22886 |
| RABV85 | Light chain variable region | | CN101337990 SEQ I DNO: 10 | 22887 |
| RABV86 | Light chain variable region | R8VL | CN104193823 SEQ ID NO: 4 | 22888 |
| RABV87 | Light chain variable region | R5 VL | CN104193823 SEQ ID NO: 5 | 22889 |
| RABV88 | Light chain variable region | R7 VL | CN104193823 SEQ ID NO: 6 | 22890 |
| RABV89 | Light chain variable region | R9 VL | CN104193823 SEQ ID NO: 7 | 22891 |
| RABV90 | Light chain variable region | | CN101696242 SEQ ID NO: 8 | 22892 |
| RABV91 | Light chain variable region | | CN101235086 SEQ ID NO: 39 | 22893 |

In some embodiments, the payload region of the viral particle comprises one or more nucleic acid sequences encoding one or more of the payload antibody polypeptides listed in Table 23 against Chagas Virus (CHAG1-CHAG2; SEQ ID NO: 22894-22895).

TABLE 23
Antibodies against Chagas Virus

| Antibody | | | SEQ |
|---|---|---|---|
| No. | Description | Reference Information | ID NO |
| CHAG1 | Heavy Chain Of The Fab Fragment, | Buschiazzo et al., PLoS Pathol. 8 (1), E1002474 | 22894 |
| | Trypanosoma cruzi trans-sialidase | (2012), NCBI Accession # 3OPZ_J (222aa) | |
| CHAG2 | Light chain of Fab fragment, | Buschiazzo et al., PLoS Pathog. 8 (1), E1002474 | 22895 |
| | Trypanosoma cmzi trans-sialidase | (2012), NCBI Accession # 3OPZ_N (213aa) | |

In some embodiments, the payload region of the viral particle comprises one or more nucleic acid sequences encoding one or more of the payload antibody polypeptides listed in Table 24 against Chikungunya Virus (CHIK1-CHIK6; SEQ ID NO: 22896-22901).

TABLE 24
Antibodies against Chikungunya Virus

| Antibody | | Antibody | | SEQ |
|---|---|---|---|---|
| No. | Description | Name | Reference Information | ID NO |
| CHIK1 | Heavy chain Fab | 9.8b | Sun, S. et al., Structural analyses at pseudo | 22896 |
| | fragment | | atomic resolution of Chikungunya virus and | |
| | | | antibodies show mechanisms of | |
| | | | neutralization, Elife 2, E00435 (2013), NCBI | |
| | | | Accession # 4GQ9_H (218 aa) | |
| CHIK2 | Heavy chain | 5F10F17E2 | US20130189279 SEQ ID NO: 6 | 22897 |
| | variable | | | |
| CHIK3 | Heavy chain | 8B10F8 | US20130189279 SEQ ID NO: 26 | 22898 |
| | variable | | | |
| CHIK4 | Light chain Fab | 9.8b | Sun, S. et al., Structural analyses at pseudo | 22899 |
| | fragment | | atomic resolution of Chikungunya virus and | |
| | | | antibodies show mechanisms of | |
| | | | neutralization, Elife 2, E00435 (2013), NCBI | |
| | | | Accession # 4GQ9_L (212 aa) | |
| CHIK5 | Light chain | 5F10F175E2 | US20130189279 SEQ ID NO: 8 | 22900 |
| | variable | | | |
| CHIK6 | Light chain | 8B10F8 | US20130189279 SEQ ID NO: 28 | 22901 |
| | variable | | | |

In some embodiments, the payload region of the viral particle comprises one or more nucleic acid sequences, fragment or variants thereof or encodes one or more polypeptides, fragments or variants thereof encoding antibodies described International Pub No. WO1983001785 and U.S. Pat. No. 5,827,671, the contents of each of which are herein incorporated by reference in their entirety, against the protozoan parasite Leishmania.

In some embodiments, the payload region of the viral particle comprises one or more nucleic acid sequences, fragment or variants thereof or encodes one or more polypeptides, fragments or variants thereof encoding antibodies against the Buruli ulcer Mycobacterium ulcerans), Leprosy/Hansen's disease (Mycobacterium leprae), Leishmaniasis, Cysticercosis, Dracunculiasis (Guinea Worm Disease), Echinococcosis, Fascioliasis, Human African Trypanosomiasis (African Sleeping Sickness), Lymphatic filariasis, Onchocerciasis, Schistosomiasis, Soil-transmitted Helminths (STH).

### Toxins

In some embodiments, the payload region of the viral particle comprises one or more nucleic acid sequences encoding one or more of the toxin related payload antibody polypeptides listed in Tables 2528.

In some embodiments, the payload region of the viral particle comprises one or more nucleic acid sequences encoding one or more of the payload antibody polypeptides listed in Table 25 against Ricin Toxin (RICN1-RICN20; SEQ ID NO: 22902-22921).

TABLE 25
Antibodies against Ricin Toxin

| Antibody | | Antibody | | SEQ |
|---|---|---|---|---|
| No. | Description | Name | Reference Information | ID NO |
| RICN1 | Camelid heavy-chain only | RTA: JIV-F5 | WO2015100409 SEQ ID NO: 124 | 22902 |
| RICN2 | Camelid heavy-chain only | JIV-F6 | WO2015100409 SEQ ID NO: 126 | 22903 |
| RICN3 | Camelid heavy-chain only | JIV-G 12 | WO2015100409 SEQ ID NO: 128 | 22904 |
| RICN4 | Camelid heavy-chain only | JIY-A7 | WO2015100409 SEQ ID NO: 130 | 22905 |
| RICN5 | Camelid heavy-chain only | JIY-D9 | WO2015100409 SEQ ID NO: 132 | 22906 |
| RICN6 | Camelid heavy-chain only | JIY-D10 | WO2015100409 SEQ ID NO: 134 | 22907 |
| RICN7 | Camelid heavy-chain only | JIY-El | WO2015100409 SEQ ID NO: 136 | 22908 |
| RICN8 | Camelid heavy-chain only | JIY-E3 | WO2015100409 SEQ ID NO: 138 | 22909 |
| RICN9 | Camelid heavy-chain only | JIY-E5 | WO2015100409 SEQ ID NO: 140 | 22910 |
| RICN10 | Camelid heavy-chain only | JIY-F10 | WO2015100409 SEQ ID NO: 142 | 22911 |
| RICN11 | Camelid heavy-chain only | JIY-G11 | WO2015100409 SEQ ID NO: 144 | 22912 |
| RICN12 | Camelid heavy-chain only | RTB: JIW-B1 | WO2015100409 SEQ ID NO: 146 | 22913 |
| RICN13 | Camelid heavy-chain only | JIW-C12 | WO2015100409 SEQ ID NO: 148 | 22914 |
| RICN14 | Camelid heavy-chain only | JIW-D12 | WO2015100409 SEQ ID NO: 150 | 22915 |
| RICN15 | Camelid heavy-chain only | JIW-G5 | WO2015100409 SEQ ID NO: 152 | 22916 |
| RICN16 | Camelid heavy-chain only | JIW-G 10 | WO2015100409 SEQ ID NO: 154 | 22917 |
| RICN17 | Camelid heavy-chain only | JIZ-B7 | WO2015100409 SEQ ID NO: 156 | 22918 |
| RICN18 | Camelid heavy-chain only | JIZ- B9 | WO2015100409 SEQ ID NO: 158 | 22919 |
| RICN19 | Camelid heavy-chain only | JIZ-D8 | WO2015100409 SEQ ID NO: 160 | 22920 |
| RICN20 | Camelid heavy-chain only | JIZ-G4 | WO2015100409 SEQ ID NO: 162 | 22921 |

In some embodiments, the payload region of the viral particle comprises one or more nucleic acid sequences encoding one or more of the payload antibody polypeptides listed in Table 26 against Anthrax (ANTH1-ANTH245; SEQ ID NO: 22922-23166).

TABLE 26
Antibodies against Anthrax

| Antibody | | Antibody | | SEQ |
|---|---|---|---|---|
| No. | Description | Name | Reference Information | ID NO |
| ANTH1 | Camelid heavy-chain only | JHD-B6 | WO2015100409 SEQ ID NO: 100 | 22922 |
| ANTH2 | Camelid heavy-chain only | JHE-D9 | WO2015100409 SEQ ID NO: 102 | 22923 |
| ANTH3 | Camelid heavy-chain only | JIJ-A12 | WO2015100409 SEQ ID NO: 104 | 22924 |
| ANTH4 | Camelid heavy-chain only | JIJ-B8 | WO2015100409 SEQ ID NO: 106 | 22925 |
| ANTH5 | Camelid heavy-chain only | JIJ-C11 | WO2015100409 SEQ ID NO: 108 | 22926 |
| ANTH6 | Camelid heavy-chain only | JIJ-D3 | WO2015100409 SEQ ID NO: 110 | 22927 |
| ANTH7 | Camelid heavy-chain only | JIJ-E9 | WO2015100409 SEQ ID NO: 112 | 22928 |
| ANTH8 | Camelid heavy-chain only | JIJ-F11 | WO2015100409 SEQ ID NO: 114 | 22929 |
| ANTH9 | Camelid heavy-chain only | JIK-B8 | WO2015100409 SEQ ID NO: 116 | 22930 |
| ANTH10 | Camelid heavy-chain only | JI -B 10 | WO2015100409 SEQ ID NO: 118 | 22931 |
| ANTH11 | Camelid heavy-chain only | JIK-B 12 | WO2015100409 SEQ ID NO: 120 | 22932 |
| ANTH12 | Camelid heavy-chain only | JIK-F4 | WO2015100409 SEQ ID NO: 122 | 22933 |
| ANTH13 | CDR | | WO2003063768 SEQ ID NO: 1 | 22934 |
| ANTH14 | CDR | | WO2003063768 SEQ ID NO: 2 | 22935 |
| ANTH15 | CDR | | WO2003063768 SEQ ID NO: 3 | 22936 |
| ANTH16 | Heavy chain | | U.S. Pat. No. 8,617,548 SEC ID NO: 2 | 22937 |
| ANTH17 | Heavy chain | IQNPA Lambda | U.S. Pat. No. 7,658,925 SEQ ID NO: 2 | 22938 |
| ANTH18 | Heavy chain | IQNLF Lambda | U.S. Pat. No. 7,658,925 SEQ ID NO: 6 | 22939 |
| ANTH19 | Heavy chain | 1A5 | US20090022736 SEQ ID NO: 1 | 22940 |
| ANTH20 | Heavy chain | 4A12 | US20090022736 SEQ ID NO: 3 | 22941 |
| ANTH21 | Heavy chain | 24B1 | US20090022736 SEQ ID NO: 5 | 22942 |
| ANTH22 | Heavy chain | 24G4 | US20090022736 SEQ ID NO: 7 | 22943 |
| ANTH23 | Heavy chain | 32E12 | US20090022736 SEQ ID NO: 9 | 22944 |
| ANTH24 | Heavy chain | 33F4 | US20090022736 SEQ ID NO: 11 | 22945 |
| ANTH25 | Heavy chain | scfv 2LF | EP2778173 SEQ ID NO: 9 | 22946 |
| ANTH26 | Heavy chain | | US20040258699 SEQ ID NO: 78 | 22947 |
| ANTH27 | Heavy chain | | US20040258699 SEQ ID NO: 79 | 22948 |
| ANTH28 | Heavy chain | | US20040258699 SEQ ID NO: 80 | 22949 |
| ANTH29 | Heavy chain | | US20040258699 SEQ ID NO: 81 | 22950 |
| ANTH30 | Heavy chain | | US20040258699 SEQ ID NO: 82 | 22951 |
| ANTH31 | Heavy chain | | US20040258699 SEQ ID NO: 83 | 22952 |
| ANTH32 | Heavy chain | | US20040258699 SEQ ID NO: 84 | 22953 |
| ANTH33 | Heavy chain | | US20040258699 SEQ ID NO: 85 | 22954 |
| ANTH34 | Heavy chain | | US20040258699 SEQ ID NO: 86 | 22955 |
| ANTH35 | Heavy chain | | US20040258699 SEQ ID NO: 87 | 22956 |
| ANTH36 | Heavy chain | | US20040258699 SEQ ID NO: 88 | 22957 |
| ANTH37 | Heavy chain | | US20040258699 SEQ ID NO: 89 | 22958 |
| ANTH38 | Heavy chain | | US20040258699 SEQ ID NO: 90 | 22959 |
| ANTH39 | Heavy chain | | US20040258699 SEQ ID NO: 91 | 22960 |
| ANTH40 | Heavy chain | | US20040258699 SEQ ID NO: 92 | 22961 |
| ANTH41 | Heavy chain | | US20040258699 SEQ ID NO: 93 | 22962 |
| ANTH42 | Heavy chain | | US20040258699 SEQ ID NO: 94 | 22963 |
| ANTH43 | Heavy chain | | US20040258699 SEQ ID NO: 95 | 22964 |
| ANTH44 | Heavy chain | | US20040258699 SEQ ID NO: 96 | 22965 |
| ANTH45 | Heavy chain | | US20040258699 SEQ ID NO: 97 | 22966 |
| ANTH46 | Heavy chain | | US20040258699 SEQ ID NO: 98 | 22967 |
| ANTH47 | Heavy chain | | US20040258699 SEQ ID NO: 99 | 22968 |
| ANTH48 | Heavy chain | | US20040258699 SEQ ID NO: 100 | 22969 |
| ANTH49 | Heavy chain | | US20040258699 SEQ ID NO: 101 | 22970 |
| ANTH50 | Heavy chain | | US20040258699 SEQ ID NO: 102 | 22971 |
| ANTH51 | Heavy chain | | US20040258699 SEQ ID NO: 103 | 22972 |
| ANTH52 | Heavy chain | | US20040258699 SEQ ID NO: 104 | 22973 |
| ANTH53 | Heavy chain | | US20040258699 SEQ ID NO: 105 | 22974 |
| ANTH54 | Heavy chain | | US20040258699 SEQ ID NO: 106 | 22975 |
| ANTH55 | Heavy chain | | US20040258699 SEQ ID NO: 107 | 22976 |
| ANTH56 | Heavy chain | | US20040258699 SEQ ID NO: 108 | 22977 |
| ANTH57 | Heavy chain | | US20040258699 SEQ ID NO: 109 | 22978 |
| ANTH58 | Heavy chain | | US20040258699 SEQ ID NO: 110 | 22979 |
| ANTH59 | Heavy chain | | US20040258699 SEQ ID NO: 111 | 22980 |
| ANTH60 | Heavy chain | | US20040258699 SEQ ID NO: 112 | 22981 |
| ANTH61 | Heavy chain | | US20040258699 SEQ ID NO: 113 | 22982 |
| ANTH62 | Heavy chain | | US20040258699 SEQ ID NO: 114 | 22983 |
| ANTH63 | Heavy chain | | US20040258699 SEQ ID NO: 115 | 22984 |
| ANTH64 | Heavy chain | | US20040258699 SEQ ID NO: 116 | 22985 |
| ANTH65 | Heavy chain | | US20040258699 SEQ ID NO: 117 | 22986 |
| ANTH66 | Heavy chain | | US20040258699 SEQ ID NO: 118 | 22987 |
| ANTH67 | Heavy chain and light chain | 14B7 scFV | U.S. Pat. No. 7,902,344; | 22988 |
| | variable region | | U.S. Pat. No. 6,916,474 SEQ ID NO: 21 | |
| ANTH68 | Heavy chain fd region | W1 | U.S. Pat. No. 8,685,396 SEQ ID NO: 1 | 22989 |
| ANTH69 | Heavy chain fd region | W2 | U.S. Pat. No. 8,685,396 SEQ ID NO: 17 | 22990 |
| ANTH70 | Heavy chain | W5 | U.S. Pat. No. 8,685,396 SEQ ID NO: 33 | 22991 |
| ANTH71 | Heavy chain | A63-6 | U.S. Pat. No. 8,685,396 SEQ ID NO: 34 | 22992 |
| ANTH72 | Heavy chain | F3-6 | U.S. Pat. No. 8,685,396 SEQ ID NO: 35 | 22993 |
| ANTH73 | Heavy chain | F5-1 | U.S. Pat. No. 8,685,396 SEQ ID NO: 36 | 22994 |
| ANTH74 | Heavy chain variable region | ETI-204 | US2010156196 SEQ ID NO: 1 | 22995 |
| ANTH75 | Heavy chain variable region | 6.20 | WO2015107307 SEQ ID NO: 1 | 22996 |
| ANTH76 | Heavy chain variable region | 33PA83 | WO2009071860 SEQ ID NO: 1 | 22997 |
| ANTH77 | Heavy chain variable region | anti-γDPGA | U.S. Pat. No. 8,501,182 SEQ ID NO: 1 | 22998 |
| | | antibody | | |
| ANTH78 | Heavy chain variable region | 4C | U.S. Pat. No. 8,501,182 SEQ ID NO: 3 | 22999 |
| ANTH79 | Heavy chain variable region | 11D | U.S. Pat. No. 8,501,182 SEQ ID NO: 5 | 23000 |
| ANTH80 | Heavy chain variable region | F20G75 | WO2007131363 SEQ ID NO: 16 | 23001 |
| ANTH81 | Heavy chain variable region | F20G76 | WO2007131363 SEQ ID NO: 18 | 23002 |
| ANTH82 | Heavy chain variable region | F20G77 | WO2007131363 SEQ ID NO: 20 | 23003 |
| ANTH83 | Heavy chain variable region | V2 variant | U.S. Pat. No. 8,507,655 SEQ ID NO: 7 | 23004 |
| ANTH84 | Heavy chain variable region | 6.20 variant | U.S. Pat. No. 8,507,655 SEQ ID NO: 9 | 23005 |
| ANTH85 | Heavy chain variable region | J24.15 variant | U.S. Pat. No. 8,507,653 SEQ ID NO: 11 | 23006 |
| ANTH86 | Heavy chain variable region | J24.7 variant | U.S. Pat. No. 8,507,655 SEQ ID NO: 13 | 23007 |
| ANTH87 | Heavy chain variable region | V2 variant human | U.S. Pat. No. 8,507,655 SEQ ID NO: 15 | 23008 |
| ANTH88 | Heavy chain variable region | 6.20 variant | U.S. Pat. No. 8,507,655 SEQ ID NO: 17 | 23009 |
| | | human | | |
| ANTH89 | Heavy chain variable region | J24.15 variant | U.S. Pat. No. 8,507,655 SEQ ID NO: 19 | 23010 |
| | | human | | |
| ANTH90 | Heavy chain variable region | J24.7 variant | U.S. Pat. No. 8,507,655 SEQ ID NO: 21 | 23011 |
| | | human | | |
| ANTH91 | Heavy chain variable region | HuMab 5E8 | U.S. Pat. No. 8,404,820 SEQ ID NO: 2 | 23012 |
| ANTH92 | Heavy chain variable region | HnMab 2D5 | U.S. Pat. No. 8,404,820 SEQ ID NO: 8 | 23013 |
| ANTH93 | Heavy chain variable region | HuMab 2H4 | U.S. Pat. No. 8,404,820 SEQ ID NO: 12 | 23014 |
| ANTH94 | Heavy chain variable region | HuMab 5D5- | U.S. Pat. No. 8,404,820 SEQ ID NO: 16 | 23015 |
| | | 2E10 | | |
| ANTH95 | Heavy chain variable region | 13E3 | U.S. Pat. No. 8,309,090 SEQ ID NO: 2 | 23016 |
| ANTH96 | Heavy chain variable region | 3E1 | U.S. Pat. No. 8,309,090 SEQ ID NO: 6 | 23017 |
| ANTH97 | Heavy chain variable region | KCTC 10756BP | U.S. Pat. No. 8,268,316 SEQ ID NO: 2 | 23018 |
| ANTH98 | Heavy chain variable region | M18 scFv | U.S. Pat. No. 7,902,344; | 23019 |
| | | | U.S. Pat. No. 6,916,474 SEQ ID NO: 23 | |
| ANTH99 | Heavy chain variable region | 21D9 MAb | U.S. Pat. No. 7,442,373 SEQ ID NO: 2 | 23020 |
| ANTH100 | Heavy chain variable region | 1C6 Mab | U.S. Pat. No. 7,442,373 SEQ ID NO: 6 | 23021 |
| ANTH101 | Heavy chain variable region | 4H7 Mab | U.S. Pat. No. 7,442,373 SEQ ID NO: 10 | 23022 |
| ANTH102 | Heavy chain variable region | 22G12 Mab | U.S. Pat. No. 7,442,373 SEQ ID NO: 14 | 23023 |
| ANTH103 | Heavy chain variable region | monoclonal | WO1999055842 SEQ ID NO: 20 | 23024 |
| | | antibody 9-1 | | |
| ANTH104 | Heavy chain variable region | monoclonal | WO1999055842 SEQ ID NO: 21 | 23025 |
| | | antibody 7-1 | | |
| ANTH105 | Heavy chain variable region | monoclonal | WO1999055842 SEQ ID NO: 22 | 23026 |
| | | antibody 24-2 | | |
| ANTH106 | Heavy chain variable region | monoclonal | WO1999055842 SEQ ID NO: 23 | 23027 |
| | | antibody 21-4 | | |
| ANTH107 | Heavy chain variable region | monoclonal | WO1999055842 SEQ ID NO: 24 | 23028 |
| | | antibody 10-2 | | |
| ANTH108 | Heavy chain variable region | monoclonal | WO1999055842 SEQ ID NO: 25 | 23029 |
| | | antibody 22-1 | | |
| ANTH109 | Heavy chain variable region | monoclonal | WO1999055842 SEQ ID NO: 26 | 23030 |
| | | antibody 13-3 | | |
| ANTH110 | Heavy chain variable region | monoclonal | WO1999055842 SEQ ID NO: 27 | 23031 |
| | | antibody 8-3 | | |
| ANTH111 | Heavy chain variable region | monoclonal | WO1999055842 SEQ ID NO: 29 | 23032 |
| | | antibody 6-1 | | |
| ANTH112 | Heavy chain variable region | monoclonal | WO1999055842 SEQ ID NO: 30 | 23033 |
| | | antibody 3-1 | | |
| ANTH113 | Heavy chain variable region, | EF12A | U.S. Pat. No. 8,961,975 SEQ ID NO: 51 | 23034 |
| | Edema factor binding | | | |
| ANTH114 | Heavy chain variable region, | EF13D | U.S. Pat. No. 8,961,975 SEQ ID NO: 33 | 23035 |
| | Edema factor binding | | | |
| ANTH115 | Heavy chain variable region, | EF14H | U.S. Pat. No. 8,961,975 SEQ ID NO: 52 | 23036 |
| | Edema factor binding | | | |
| ANTH116 | Heavy chain variable region, | EF15A | U.S. Pat. No. 8,961,975 SEQ ID NO: 53 | 23037 |
| | Edema factor binding | | | |
| ANTH117 | Heavy chain variable region, | LF9D | U.S. Pat. No. 8,961,975 SEQ ID NO: 49 | 23038 |
| | Lethal factor | | | |
| ANTH118 | Heavy chain variable region, | LF10E | U.S. Pat. No. 8,961,975 SEQ ID NO: 1 | 23039 |
| | Lethal factor | | | |
| ANTH119 | Heavy chain, Antibody | Obiltoxaximab | | 23040 |
| | against inhalational anthrax | | | |
| ANTH120 | Kappa light chain | | US20040258699 SEQ ID NO: 19 | 23041 |
| ANTH121 | Kappa light chain | | US20040258699 SEQ ID NO: 20 | 23042 |
| ANTH122 | Kappa light chain | | US20040258699 SEQ ID NO: 21 | 23043 |
| ANTH123 | Kappa tight chain | | US20040258699 SEQ ID NO: 22 | 23044 |
| ANTH124 | Kappa light chain | | US20040258699 SEQ ID NO: 23 | 23045 |
| ANTH125 | Kappa light chain | | US20040258699 SEQ ID NO: 24 | 23046 |
| ANTH126 | Kappa light chain | | US20040258699 SEQ ID NO: 25 | 23047 |
| ANTH127 | Kappa light chain | | US20040258699 SEQ ID NO: 26 | 23048 |
| ANTH128 | Kappa tight chain | | US20040258699 SEQ ID NO: 39 | 23049 |
| ANTH129 | Kappa light chain | | US20040258699 SEQ ID NO: 40 | 23050 |
| ANTH130 | Kappa light chain | | US20040258699 SEQ ID NO: 41 | 23051 |
| ANTH131 | Kappa light chain | | US20040258699 SEQ ID NO: 42 | 23052 |
| ANTH132 | Kappa light chain | | US20040258699 SEQ ID NO: 43 | 23053 |
| ANTH133 | Kappa light chain | | US20040258699 SEQ ID NO: 44 | 23054 |
| ANTH134 | Kappa light chain | | US20040258699 SEQ ID NO: 45 | 23055 |
| ANTH135 | Kappa light chain | | US20040258699 SEQ ID NO: 46 | 23056 |
| ANTH136 | Kappa light chain | | US20040258699 SEQ ID NO: 47 | 23057 |
| ANTH137 | Kappa light chain | | US20040258699 SEQ ID NO: 48 | 23058 |
| ANTH138 | Kappa light chain | | US20040258699 SEQ ID NO: 49 | 23059 |
| ANTH139 | Kappa light chain | | US20040258699 SEQ ID NO: 50 | 23060 |
| ANTH140 | Kappa light chain | | US20040258699 SEQ ID NO: 51 | 23061 |
| ANTH141 | Kappa light chain | | US20040258699 SEQ ID NO: 52 | 23062 |
| ANTH142 | Kappa light chain | | US20040258699 SEQ ID NO: 53 | 23063 |
| ANTH143 | Kappa light chain | | US20040258699 SEQ ID NO: 54 | 23064 |
| ANTH144 | Kappa light chain | | US20040258699 SEQ ID NO: 55 | 23065 |
| ANTH145 | Kappa light chain | | US20040258699 SEQ ID NO: 56 | 23066 |
| ANTH146 | Kappa light chain | | US20040258699 SEQ ID NO: 57 | 23067 |
| ANTH147 | Kappa light chain | | US20040258699 SEQ ID NO: 58 | 23068 |
| ANTH148 | Kappa light chain | | US20040258699 SEQ ID NO: 59 | 23069 |
| ANTH149 | Kappa light chain | | US20040258699 SEQ ID NO: 60 | 23070 |
| ANTH150 | Kappa light chain | | US20040258699 SEQ ID NO: 61 | 23071 |
| ANTH151 | Lambda light chain | | US20040258699 SEQ ID NO: 27 | 23072 |
| ANTH152 | Lambda light chain | | US20040258699 SEQ ID NO: 28 | 23073 |
| ANTH153 | Lambda light chain | | US20040258699 SEQ ID NO: 29 | 23074 |
| ANTH154 | Lambda light chain | | US20040258699 SEQ ID NO: 30 | 23075 |
| ANTH155 | Lambda light chain | | US20040258699 SEQ ID NO: 31 | 23076 |
| ANTH156 | Lambda light chain | | US20040258699 SEQ ID NO: 32 | 23077 |
| ANTH157 | Lambda light chain | | US20040258699 SEQ ID NO: 33 | 23078 |
| ANTH158 | Lambda light chain | | US20040258699 SEQ ID NO: 34 | 23079 |
| ANTH159 | Lambda light chain | | US20040258699 SEQ ID NO: 35 | 23080 |
| ANTH160 | Lambda tight chain | | US20040258699 SEQ ID NO: 36 | 23081 |
| ANTH161 | Lambda light chain | | US20040258699 SEQ ID NO: 37 | 23082 |
| ANTH162 | Lambda light chain | | US20040258699 SEQ ID NO: 38 | 23083 |
| ANTH163 | Lambda light chain | | US20040258699 SEQ ID NO: 62 | 23084 |
| ANTH164 | Lambda light chain | | US20040258699 SEQ ID NO: 63 | 23085 |
| ANTH165 | Lambda light chain | | US20040258699 SEQ ID NO: 64 | 23086 |
| ANTH166 | Lambda light chain | | US20040258699 SEQ ID NO: 65 | 23087 |
| ANTH167 | Lambda light chain | | US20040258699 SEQ ID NO: 66 | 23088 |
| ANTH168 | Lambda light chain | | US20040258699 SEQ ID NO: 67 | 23089 |
| ANTH169 | Lambda light chain | | US20040258699 SEQ ID NO: 68 | 23090 |
| ANTH170 | Lambda light chain | | US20040258699 SEQ ID NO: 69 | 23091 |
| ANTH171 | Lambda light chain | | US20040258699 SEQ ID NO: 70 | 23092 |
| ANTH172 | Lambda light chain | | US20040258699 SEQ ID NO: 71 | 23093 |
| ANTH173 | Lambda light chain | | US20040258699 SEQ ID NO: 72 | 23094 |
| ANTH174 | Lambda light chain | | US20040258699 SEQ ID NO: 73 | 23095 |
| ANTH175 | Lambda light chain | | US20040258699 SEQ ID NO: 74 | 23096 |
| ANTH178 | Lambda light chain | | US20040258699 SEQ ID NO: 75 | 23097 |
| ANTH177 | Lambda light chain | | US20040258699 SEQ ID NO: 76 | 23098 |
| ANTH178 | Lambda light chain | | US20040258699 SEQ ID NO: 77 | 23099 |
| ANTH179 | Light chain | | U.S. Pat. No. 8,617,548 SEQ ID NO: 1 | 23100 |
| ANTH180 | Light chain | IQNPA Lkappa | U.S. Pat. No. 7,658,925 SEQ ID NO: 4 | 23101 |
| ANTH181 | Light chain | IQNLF Lkappa | U.S. Pat. No. 7,658,925 SEQ ID NO: 8 | 23102 |
| ANTH182 | Light chain | 1A5 | US20090022736 SEQ ID NO: 2 | 23103 |
| ANTH183 | Light chain | 4A12 | US20090022736 SEQ ID NO: 4 | 23104 |
| ANTH184 | Light chain | 24B1 | US20090022736 SEQ ID NO: 6 | 23105 |
| ANTH185 | Light chain | 24G4 | US20090022736 SEQ ID NO: 8 | 23106 |
| ANTH186 | Light chain | ′32E12 | US20090022736 SEQ ID NO: 10 | 23107 |
| ANTH187 | Light chain | 33F4 | US20090022736 SEQ ID NO: 12 | 23108 |
| ANTH188 | Light chain | scFv 2LF | EP2778173 SEQ ID NO: 6 | 23109 |
| ANTH189 | Light chain | Obiltoxaximab | | 23110 |
| ANTH190 | Light chain region | W1 | U.S. Pat. No. 8,685,396 SEQ ID NO: 9 | 23111 |
| ANTH191 | Light chain region | W2 | U.S. Pat. No. 8,685,396 SEQ ID NO: 25 | 23112 |
| ANTH192 | Light chain region | W5 | U.S. Pat. No. 8,685,396 SEQ ID NO: 37 | 23113 |
| ANTH193 | Light chain region | A63-6 | U.S. Pat. No. 8,685,396 SEQ ID NO: 38 | 23114 |
| ANTH194 | Light chain region | F3-6 | U.S. Pat. No. 8,685,396 SEQ ID NO: 39 | 23115 |
| ANTH195 | Light chain region | F5-1 | U.S. Pat. No. 8,685,396 SEQ ID NO: 40 | 23116 |
| ANTH196 | Light chain variable region | LF11H | U.S. Pat. No. 8,961,975 SEQ ID NO: 25 | 23117 |
| ANTH197 | Light chain variable region | LF9D | U.S. Pat. No. 8,961,975 SEQ ID NO: 17 | 23118 |
| ANTH198 | Light chain variable region | LF10E | U.S. Pat. No. 8,961,975 SEQ ID NO: 9 | 23119 |
| ANTH199 | Light chain variable region | 6.20 | WO2015107307 SEQ ID NO: 2 | 23120 |
| ANTH200 | Light chain variable region | 35PA83 | WO2009071860 SEQ ID NO: 2 | 23121 |
| ANTH201 | Light chain variable region | anti-γDPGA | U.S. Pat. No. 8,501,182 SEQ ID NO: 2 | 23122 |
| | | antibody | | |
| ANTH202 | Light chain variable region | 4C | U.S. Pat. No. 8,501,182 SEQ ID NO: 4 | 23123 |
| ANTH203 | Light chain variable region | 11D | U.S. Pat. No. 8,501,182 SEQ ID NO: 6 | 23124 |
| ANTH204 | Light chain variable region | F20G75 | WO2007131363 SEQ ID NO: 10 | 23125 |
| ANTH205 | Light chain variable region | F20G76 | WO2007131363 SEQ ID NO: 12 | 23126 |
| ANTH206 | Light chain variable region | F20G77 | WO2007131363 SEQ ID NO: 14 | 23127 |
| ANTH207 | Light chain variable region | V2 variant | U.S. Pat. No. 8,507,655 SEQ ID NO: 8 | 23128 |
| ANTH208 | Light chain variable region | 6.20 variant | U.S. Pat. No. 8,507,655 SEQ ID NO: 10 | 23129 |
| ANTH209 | Light chain variable region | J24.15 variant | U.S. Pat. No. 8,507,655 SEQ ID NO: 12 | 23130 |
| ANTH210 | Light chain variable region | J24.7 variant | U.S. Pat. No. 8,507,655 SEQ ID NO: 14 | 23131 |
| ANTH211 | Light chain variable region | V2 variant human | U.S. Pat. No. 8,507,655 SEQ ID NO: 16 | 23132 |
| ANTH212 | Light chain variable region | 6.20 variant | U.S. Pat. No. 8,507,655 SEQ ID NO: 18 | 23133 |
| | | human | | |
| ANTH213 | Light chain variable region | J24.15 variant | U.S. Pat. No. 8,507,655 SEQ ID NO: 20 | 23134 |
| | | human | | |
| ANTH214 | Light chain variable region | J24.7 variant | U.S. Pat. No. 8,507,655 SEQ ID NO: 22 | 23135 |
| | | human | | |
| ANTH215 | Light chain variable region | HuMab 5E8 | U.S. Pat. No. 8,401,820 SEQ ID NO: 4 | 23136 |
| | | (Major) | | |
| ANTH216 | Light chain variable region | HuMab 5E8 | U.S. Pat. No. 8,404,820 SEQ ID NO: 6 | 23137 |
| | | (Minor) | | |
| ANTH217 | Light chain variable region | HuMab 2P5 | U.S. Pat. No. 8,404,820 SEQ ID NO: 10 | 23138 |
| ANTH218 | Light chain variable region | HuMab 2H4 | U.S. Pat. No. 8,404,820 SEQ ID NO: 14 | 23139 |
| ANTH219 | Light chain variable region | HuMab 5D5- | U.S. Pat. No. 8,404,820 SEQ ID NO: 18 | 23140 |
| | | 2E10 | | |
| ANTH220 | Light chain variable region | 13E3 | U.S. Pat. No. 8,309,090 SEQ ID NO: 4 | 23141 |
| ANTH221 | Light chain variable region | 3E1 | U.S. Pat. No. 8,309,090 SEQ ID NO: 8 | 23142 |
| ANTH222 | Light chain variable region | KCTC 10756BP | U.S. Pat. No. 8,268,316 SEQ ID NO: 7 | 23143 |
| ANTH223 | Light chain variable region | modified M18 | U.S. Pat. No. 7,902,344; | 23144 |
| | | sequence | U.S. Pat. No. 6,916,474 SEQ ID NO: 25 | |
| ANTH224 | Light chain variable region | 21D9 MAb | U.S. Pat. No. 7,442,373 SEQ ID NO: 4 | 23145 |
| ANTH225 | Light chain variable region | 1C6 Mab | U.S. Pat. No. 7,442,373 SEQ ID NO: 8 | 23146 |
| ANTH226 | Light chain variable region | 4H7 Mab | U.S. Pat. No. 7,442,373 SEQ ID NO: 12 | 23147 |
| ANTH227 | Light chain variable region | 22G12 Mab | U.S. Pat. No. 7,442,373 SEQ ID NO: 16 | 23148 |
| ANTH228 | Light chain variable region | ETI-204 | US20120156196 SEQ ID NO: 2 | 23149 |
| | antibody against anthrax toxin, | | | |
| ANTH229 | Light chain variable region, | EF12A | U.S. Pat. No. 8,961,975 SEQ ID NO: 54 | 23150 |
| | Edema factor | | | |
| ANTH230 | Light chain variable region, | EF13D | U.S. Pat. No. 8,961,975 SEQ ID NO: 41 | 23151 |
| | Edema factor | | | |
| ANTH231 | Light chain variable region, | EF14H | U.S. Pat. No. 8,961,973 SEQ ID NO: 55 | 23152 |
| | Edema factor | | | |
| ANTH232 | Light chain variable region, | EP15A | U.S. Pat. No. 8,961,975 SEQ ID NO: 56 | 23153 |
| | Edema factor | | | |
| ANTH233 | Scfv | PWB2447 scFv | U.S. Pat. No. 7,601,351; | 23154 |
| | | | U.S. Pat. No. 7,906,119; | |
| | | | US20110189197 SEQ ID NO: 48 | |
| ANTH234 | Scfv | PWC2004 scFv | U.S. Pat. No. 7,601,351; | 23155 |
| | | | U.S. Pat. No. 7,906,119; | |
| | | | US20110189197 SEQ ID NO: 49 | |
| ANTH235 | Scfv | PWD0283 scFv | U.S. Pat. No. 7,601,351; | 23156 |
| | | | U.S. Pat. No. 7,906,119; | |
| | | | US20110189197 SEQ ID NO: 50 | |
| ANTH236 | Scfv | PWP0323 scFv | U.S. Pat. No. 7,601,351; | 23157 |
| | | | U.S. Pat. No. 7,906,119; | |
| | | | US20110189197 SEQ ID NO: 51 | |
| ANTH237 | Scfv | PWD0422 scFv | U.S. Pat. No. 7,601,351; | 23158 |
| | | | U.S. Pat. No. 7,906,119; | |
| | | | US20110189197 SEQ ID NO: 52 | |
| ANTH238 | Scfv | PWD0587 scFv | U.S. Pat. No. 7,601,351; | 23159 |
| | | | U.S. Pat. No. 7,906,119; | |
| | | | US20110189197 SEQ ID NO: 53 | |
| ANTH239 | Scfv | PWD0791 scFv | U.S. Pat. No. 7,601,351; | 23160 |
| | | | U.S. Pat. No. 7,906,119; | |
| | | | US20110189197 SEQ ID NO: 54 | |
| ANTH240 | Scfv | PHP2222 scFv | U.S. Pat. No. 7,601,351; | 23161 |
| | | | U.S. Pat. No. 7,906,119; | |
| | | | US20110189197 SEQ ID NO: 55 | |
| ANTH241 | Scfv | PHD2581 scFv | U.S. Pat. No. 7,601,351; | 23162 |
| | | | U.S. Pat. No. 7,906,119; | |
| | | | US20110189197 SEQ ID NO: 56 | |
| ANTH242 | | Abthrax | US20120136196 SEQ ID NO: 48 | 23163 |
| ANTH243 | | Abthrax | US20120156196 SEQ ID NO: 49 | 23164 |
| ANTH244 | | | WO2003063768 SEQ ID NO: 4 | 23165 |
| ANTH245 | | | WO2003063768 SEQ ID NO: 5 | 23166 |

In some embodiments, the payload region of the viral particle comprises one or more nucleic acid sequences encoding one or more of the payload antibody polypeptides listed in Table 27 against Botulinum Toxin (BOTT1-BOTT30; SU) ID NO: 23167-23196).

TABLE 27
Antibodies against Botulinum Toxin

| | | | | SEQ |
|---|---|---|---|---|
| Antibody | | Antibody | | ID |
| No. | Description | Name | Reference Information | NO |
| BOTT1 | Heavy-chain-only | | US20130058962 SEQ ID NO: 56 | 23167 |
| BOTT2 | Heavy-chain-only | | US20130058962 SEQ ID NO: 57 | 23168 |
| BOTT3 | Heavy-chain-only | | US20130058962 SEQ ID NO: 58 | 23169 |
| BOTT4 | Heavy-chain only binding | JDA-D12 | WO2015100409 SEQ ID NO: 20 | 23170 |
| | agents specific to BoNT/A | | | |
| | holotoxin | | | |
| BOTT5 | Heavy-chain only binding | JDQ-A5 | WO2015100409 SEQ ID NO: 22 | 23171 |
| | agents specific to BoNT/A | | | |
| | holotoxin | | | |
| BOTT6 | Heavy-chain only binding | JDQ-B5 | WO2015100409 SEQ ID NO: 24 | 23172 |
| | agents specific to BoNT/A | | | |
| | holotoxin | | | |
| BOTT7 | Heavy-chain only binding | JDQ-C2 | WO2015100409 SEQ ID NO: 26 | 23173 |
| | agents specific to BoNT/A | | | |
| | holotoxin | | | |
| BOTT8 | Heavy-chain only binding | JDQ-F 12 | WO2015100409 SEQ ID NO: 28 | 23174 |
| | agents specific to BoNT/A | | | |
| | holotoxin | | | |
| BOTT9 | Heavy-chain only binding | JDQ-G5 | WO2015100409 SEQ ID NO: 30 | 23175 |
| | agents specific to BoNT/A | | | |
| | holotoxin | | | |
| BOTT10 | Heavy-chain only binding | JDQ-H7 | WO2015100409 SEQ ID NO: 32 | 23176 |
| | agents specific to BoNT/A | | | |
| | holotoxin | | | |
| BOTT11 | Heavy-chain only binding | JEQ-A5 | WO2015100409 SEQ ID NO: 34 | 23177 |
| | agents specific to BoNT/A | | | |
| | holotoxin | | | |
| BOTT12 | Heavy-chain only binding | JEQ-H11 | WO2015100409 SEQ ID NO: 36 | 23178 |
| | agents specific to BoNT/A | | | |
| | holotoxin | | | |
| BOTT13 | Heavy-chain only binding agent | E-9 | WO2015100409 SEQ ID NO: 38 | 23179 |
| BOTT14 | Heavy-chain only binding agent | B2 | WO2015100409 SEQ ID NO: 40 | 23180 |
| BOTT15 | Heavy-chain only binding agent | C5 | WO2015100409 SEQ ID NO: 42 | 23181 |
| BOTT16 | Heavy-chain only binding agent | F9 | WO2015100409 SEQ ID NO: 44 | 23182 |
| BOTT17 | Heavy-chain only binding agent | heavy-chain only | WO2015100409 SEQ ID NO: 46 | 23183 |
| | | binding agent | | |
| BOTT18 | Heavy-chain only binding agent | heavy-chain only | WO2015100409 SEQ ID NO: 48 | 23184 |
| | with tag | binding agent | | |
| | | with tag | | |
| BOTT19 | Heavy-chain only binding agent | heavy-chain only | WO2015100409 SEQ ID NO: 50 | 23185 |
| | with tag | binding agent | | |
| | | with tag | | |
| BOTT20 | Heavy-chain only dimer | heavy-chain only | WO2015100409 SEQ ID NO: 52 | 23186 |
| | binding agent with two tags | dimer binding | | |
| | | agent with two | | |
| | | tags | | |
| BOTT21 | Recombinant camelid heavy- | H7 | WO2015100409 SEQ ID NO: 56 | 23187 |
| | chain-only antibody | | | |
| BOTT22 | Recombinant camelid heavy- | B5 | WO2015100409 SEQ ID NO: 57 | 23188 |
| | chain-only antibody | | | |
| BOTT23 | Recombinant camelid heavy- | | WO2015100409 SEQ ID NO: 58 | 23189 |
| | chain-only antibody | | | |
| BOTT24 | Scfv | scFv#2 | WO2015100409 SEQ ID NO: 2 | 23190 |
| BOTT25 | Scfv | scFv#3 | WO2015100409 SEQ ID NO: 4 | 23191 |
| BOTT26 | Scfv | scFv#7 | WO2015100409 SEQ ID NO: 6 | 23192 |
| BOTT27 | Scfv | scFv#8 | WO2015100409 SEQ ID NO: 8 | 23193 |
| BOTT28 | Scfv | scFv#21 | WO2015100409 SEQ ID NO: 10 | 23194 |
| BOTT29 | Scfv | scFv#E | WO2015100409 SEQ ID NO: 12 | 23195 |
| BOTT30 | Scfv | scFv#7-2E | WO2015100409 SEQ ID NO: 14 | 23196 |

In some embodiments, the payload region of the viral particle comprises one or more nucleic acid sequences encoding one or more of the payload antibody polypeptides listed in Table 28 against Shiga Toxin (SHIG1-SHIG71; SEQ ID NO: 23197-23267).

TABLE 28
Antibodies against Shiga Toxin

| | | | | SEQ |
|---|---|---|---|---|
| Antibody | | Antibody | | ID |
| No. | Description | Name | Reference Information | NO |
| SHIG1 | Camelid heavy-chain only | JET-H12 | WO2015100409 SEQ ID NO: 96 | 23197 |
| SHIG2 | Camelid heavy-chain only | JFG-H6 | WO2015100409 SEQ ID NO: 98 | 23198 |
| SHIG3 | Heavy chain | | US2014013548 SEQ ID NO: 44 | 23199 |
| SHIG4 | Heavy chain | | US2014013548 SEQ ID NO: 21 | 23200 |
| SHIG5 | Heavy chain of cαstx1 | Shigamab | US20120195891 SEQ ID NO: 1 | 23201 |
| SHIG6 | Heavy chain of cαstx1 | Shigamab | US20120195891 SEQ ID NO: 2 | 23202 |
| SHIG7 | Heavy chain of cαstx2 | Shigamab | US20120195891 SEQ ID NO: 3 | 23203 |
| SHIG8 | Heavy chain of cαstx2 | Shigamab | US20120195891 SEQ ID NO: 4 | 23204 |
| SHIG9 | Heavy chain single domain | | WO2014191904 SEQ ID NO: 7 | 23205 |
| SHIG10 | Heavy chain single domain | | WO2014191904 SEQ ID NO: 8 | 23206 |
| SHIG11 | Heavy chain single domain | | WO2014191904 SEQ ID NO: 9 | 23207 |
| SHIG12 | Heavy chain single domain | | WO2014191904 SEQ ID NO: 10 | 23208 |
| SHIG13 | Heavy chain single domain | | WO2014191904 SEQ ID NO: 11 | 23209 |
| SHIG14 | Heavy chain single domain | | WO2014191904 SEQ ID NO: 12 | 23210 |
| SHIG15 | Heavy chain single domain | | WO2014191904 SEQ ID NO: 13 | 23211 |
| SHIG16 | Heavy chain single domain | | WO2014191904 SEQ ID NO: 14 | 23212 |
| SHIG17 | Heavy chain single domain | | WO2014191904 SEQ ID NO: 15 | 23213 |
| SHIG18 | Heavy chain single domain | | WO2014191904 SEQ ID NO: 16 | 23214 |
| SHIG19 | Heavy chain single domain | | WO2014191904 SEQ ID NO: 17 | 23215 |
| SHIG20 | Heavy chain single domain | | WO2014191904 SEQ ID NO: 18 | 23216 |
| SHIG21 | Heavy chain single domain | | WO2014191904 SEQ ID NO: 19 | 23217 |
| SHIG22 | Heavy chain single domain | | WO2014191904 SEQ ID NO: 20 | 23218 |
| SHIG23 | Heavy chain single domain | | WO2014191904 SEQ ID NO: 21 | 23219 |
| SHIG24 | Heavy chain single domain | | WO2014191904 SEQ ID NO: 22 | 23220 |
| SHIG25 | Heavy chain single domain | | WO2014191904 SEQ ID NO: 23 | 23221 |
| SHIG26 | Heavy chain single domain | | WO2014191904 SEQ ID NO: 24 | 23222 |
| SHIG27 | Heavy chain single domain | | WO2014191904 SEQ ID NO: 25 | 23223 |
| SHIG28 | Heavy chain single domain | | WO2014191904 SEQ ID NO: 26 | 23224 |
| SHIG29 | Heavy chain single domain | | WO2014191904 SEQ ID NO: 27 | 23225 |
| SHIG30 | Heavy chain single domain | | WO2014191904 SEQ ID NO: 28 | 23226 |
| SHIG31 | Heavy chain single domain | | WO2014191904 SEQ ID NO: 29 | 23227 |
| SHIG32 | Heavy chain single domain | | WO2014191904 SEQ ID NO: 30 | 23228 |
| SHIG33 | Heavy chain single domain | | WO2014191904 SEQ ID NO: 31 | 23229 |
| SHIG34 | Heavy chain single domain | | WO2014191904 SEQ ID NO: 32 | 23230 |
| SHIG35 | Heavy chain single domain | | WO2014191904 SEQ ID NO: 33 | 23231 |
| SHIG36 | Heavy chain single domain | | WO2014191904 SEQ ID NO: 34 | 23232 |
| SHIG37 | Heavy chain single domain | | WO2014191904 SEQ ID NO: 35 | 23233 |
| SHIG38 | Heavy chain single domain | | WO2014191904 SEQ ID NO: 36 | 23234 |
| SHIG39 | Heavy chain single domain | | WO2014191904 SEQ ID NO: 37 | 23235 |
| SHIG40 | Heavy chain single domain | | WO2014191904 SEQ ID NO: 38 | 23236 |
| SHIG41 | Heavy chain single domain | | WO2014191904 SEQ ID NO: 39 | 23237 |
| SHIG42 | Heavy chain single domain | | WO2014191904 SEQ ID NO: 40 | 23238 |
| SHIG43 | Heavy chain single domain | | WO2014191904 SEQ ID NO: 41 | 23239 |
| SHIG44 | Heavy chain single domain | | WO2014191904 SEQ ID NO: 42 | 23240 |
| SHIG45 | Heavy chain single domain | | WO2014191904 SEQ ID NO: 43 | 23241 |
| SHIG46 | Heavy chain single domain | | WO2014191904 SEQ ID NO: 44 | 23242 |
| SHIG47 | Heavy chain single domain | | WO2014191904 SEQ ID NO: 45 | 23243 |
| SHIG48 | Heavy chain single domain | | WO2014191904 SEQ ID NO: 46 | 23244 |
| SHIG49 | Heavy chain single domain | | WO2014191904 SEQ ID NO: 47 | 23245 |
| SHIG50 | Heavy-chain-only | | US20130058962 SEQ ID NO: 77 | 23246 |
| SHIG51 | Heavy-chain-only | | US20130058962 SEQ ID NO: 78 | 23247 |
| SHIG52 | Heavy-chain-only | | US20130058962 SEQ ID NO: 79 | 23248 |
| SHIG53 | Heavy-chain-only | | US20130058962 SEQ ID NO: 80 | 23249 |
| SHIG54 | Heavy-chain-only | | US20130058962 SEQ ID NO: 81 | 23250 |
| SHIG55 | Heavy-chain-only | | US20130058962 SEQ ID NO: 82 | 23251 |
| SHIG56 | Heavy-chain-only | | US20130058962 SEQ ID NO: 83 | 23252 |
| SHIG57 | Heavy-chain-only | | US20130058962 SEQ ID NO: 84 | 23253 |
| SHIG58 | Heavy-chain-only | | US20130058962 SEQ ID NO: 85 | 23254 |
| SHIG59 | Heavy-chain-only | | US20130058962 SEQ ID NO: 86 | 23255 |
| SHIG60 | Light chain | | US2014013548 SEQ ID NO: 42 | 23256 |
| SHIG61 | Light chain | | US2014013548 SEQ ID NO: 19 | 23257 |
| SHIG62 | Recombinant camelid heavy- | JET-A9 | WO2015100409 SEQ ID NO: 77 | 23258 |
| | chain-only antibody, STX1 | | | |
| SHIG63 | Recombinant camelid heavy- | JGG-D4 | WO2015100409 SEQ ID NO: 78 | 23259 |
| | chain-only antibody, STX1 | | | |
| SHIG64 | Recombinant camelid heavy- | JFD-A4 | WO2015100409 SEQ ID NO: 84 | 23260 |
| | chain-only antibody, STX1, | | | |
| | STX2 | | | |
| SHIG65 | Recombinant camelid heavy- | JFD-A5 | WO2015100409 SEQ ID NO: 85 | 23261 |
| | chain-only antibody, STX1, | | | |
| | STX2 | | | |
| SHIG66 | Recombinant camelid heavy- | JGG-G6 | WO2015100409 SEQ ID NO: 86 | 23262 |
| | chain-only antibody, STX1, | | | |
| | STX2 | | | |
| SHIG67 | Recombinant camelid heavy- | JEN-D10 | WO2015100409 SEQ ID NO: 79 | 23263 |
| | chain-only antibody, STX2 | | | |
| SHIG68 | Recombinant camelid heavy- | JGH-G1 | WO2015100409 SEQ ID NO: 80 | 23264 |
| | chain-only antibody, STX2 | | | |
| SHIG69 | Recombinant camelid heavy- | JEU-A6 | WO2015100409 SEQ ID NO: 81 | 23265 |
| | chain-only antibody, STX2 | | | |
| SHIG70 | Recombinant camelid heavy- | JEU-D2 | WO2015100409 SEQ ID NO: 82 | 23266 |
| | chain-only antibody, STX2 | | | |
| SHIG71 | Recombinant camelid heavy- | JGH-G9 | WO2015100409 SEQ ID NO: 83 | 23267 |
| | chain-only antibody, STX2 | | | |

In some embodiments, the payload region of the viral particle comprises one or more nucleic acid sequences, fragments or variants thereof or encodes one or more polypeptides, fragments or variants thereof described in US Pub. No. US20090280104, the contents of each of which are herein incorporated by reference in their entirety, against Shiga toxin.

### Tropical Diseases

In some embodiments, the payload region of the viral particle comprises one or more nucleic acid sequences encoding one or more of the tropical disease related payload antibody polypeptides listed in Tables 2931.

In some embodiments, the payload region of the viral particle comprises one or more nucleic acid sequences encoding one or more of the payload antibody polypeptides listed in Table 29 against Plasmodium Falciparum causing Malaria (MALA1-MALA57; SEQ ID NO: 23268-23324).

TABLE 29
Antibodies against Plasmodium Falciparum causing Malaria

| | | | | SEQ |
|---|---|---|---|---|
| Antibody | | Antibody | | ID |
| No. | Description | Name | Reference Information | NO |
| MALA1 | Heavy chain | immunoglobulin | Wajanarogana, S. et al., Construction of a | 23268 |
| | | heavy chain | human functional single-chain variable fragment | |
| | | variable region, | (scFv) antibody recognizing the malaria parasite | |
| | | partial | Plasmodium falciparum, Biotechnol. Appl. | |
| | | | Biochem. 44 (PT 1), 55-61 (2006), NCBI | |
| | | | Accession # AAX76832.1 (129aa) | |
| MALA2 | Heavy chain | anti-MSP1 | Sowa, K. M. et al., Isolation of a monoclonal | 23269 |
| | | MAD20 block2 | antibody from a malaria patient-derived phage | |
| | | ScFv Ig heavy | display library recognizing the Block 2 region | |
| | | chain variable | of Plasmodium falciparum merozoite surface | |
| | | region, partial | protein-1, Mol. Biochem. Parasitol. 112 (1), | |
| | | | 143-147 (2001), NCBI Accession #AAK08696.1 | |
| | | | (119aa) | |
| MALA3 | Heavy chain | immunoglobulin | Lundquist, R. et al., Human recombinant | 23270 |
| | | heavy chain | antibodies against Plasmodium falciparum | |
| | | variable region, | merozoite surface protein 3 cloned from | |
| | | partial | peripheral blood leukocytes of individuals with | |
| | | | immunity to malaria demonstrate antiparasitic | |
| | | | properties, Infect. Immun. 74 (6), 3222-3231, | |
| | | | (2006), NCBI Accession # AAT09786.1 | |
| | | | (113aa) | |
| MALA4 | Heavy chain | 2A10 anti- | NCBI Accession # BAK41504.1 (118aa) | 23271 |
| | variable region | malaria | | |
| | | antibody | | |
| MALA5 | Heavy chain | | U.S. Pat. No. 7,811,569 to Dziegiel; SEQ ID NO: 1 | 23272 |
| MALA6 | Heavy chain, | | U.S. Pat. No. 7,811,569 to Dziegiel; SEQ ID NO: 3 | 23273 |
| | Anti-ang-2 | | | |
| | antibody | | | |
| MALA7 | Heavy chain | | U.S. Pat. No. 7,811,569 to Dziegiel; SEQ ID NO: 5 | 23274 |
| MALA8 | Heavy chain | | US20150197562 SEQ ID NO: 14 | 23275 |
| | variable region | | | |
| MALA9 | Heavy chain | mAh 5D5 | US20150158941 SEQ ID NO: 16 | 23276 |
| | variable region | | | |
| MALA10 | Heavy chain | | US20140112930 SEQ ID NO: 18 | 23277 |
| | variable region | | | |
| MALA11 | Heavy chain | M071Xi0199 | WO2014087007; SEQ ID NO: 182 | 23278 |
| | variable region | | | |
| MALA12 | Heavy chain | M071Xi2204 | WO2014087007; SEQ ID NO: 186 | 23279 |
| | variable region | | | |
| MALA13 | Heavy chain | M071Xi0237 | WO2014087007; SEQ ID NO: 190 | 23280 |
| | variable region | | | |
| MALA14 | Heavy chain | M071Xi2127 | WO2014087007; SEQ ID NO: 194 | 23281 |
| | variable region | | | |
| MALA15 | Heavy chain | M071Xi0092 | WO2014087007; SEQ ID NO: 198 | 23282 |
| | variable region | | | |
| MALA16 | Heavy chain | M071Xi2057 | WO2014087007; SEQ ID NO: 202 | 23283 |
| | variable region | | | |
| MALA17 | Heavy chain | M070Xi3010 | WO2014087007; SEQ ID NO: 206 | 23284 |
| | variable region | | | |
| MALA18 | Heavy chain | M071Xi0227 | WO2014087007; SEQ ID NO: 210 | 23285 |
| | variable region | | | |
| MALA19 | Heavy chain | M071Xi0081 | WO2014087007; SEQ ID NO: 214 | 23286 |
| | variable region | | | |
| MALA20 | Heavy chain | M071Xi0124 | WO2014087007; SEQ ID NO: 218 | 23287 |
| | variable region | | | |
| MALA21 | Heavy chain | M036Xi0326 | WO2014087007; SEQ ID NO: 222 | 23288 |
| | variable region | | | |
| MALA22 | Heavy chain | M070Xi3195 | WO2014087007; SEQ ID NO: 226 | 23289 |
| | variable region | | | |
| MALA23 | Heavy chain | M070Xi3062 | WO2014087007; SEQ ID NO: 230 | 23290 |
| | variable region | | | |
| MALA24 | Heavy chain | M071Xi2217 | WO2014087007; SEQ ID NO: 234 | 23291 |
| | variable region | | | |
| MALA25 | Heavy chain | M036Xi0003 | WO2014087007; SEQ ID NO: 238 | 23292 |
| | variable region | | | |
| MALA26 | Heavy chain, Eba- | R217 | Chen et al., PLoS Pathol. 9 (5), E1003390 | 23293 |
| | 175 | | (2013), NCBI Accession # 4QEX_I (215aa) | |
| MALA27 | Heavy chain, Eba- | R218 | Chen et al., PLoS Pathol. 9 (5), E1003390 | 23294 |
| | 175 | | (2013), NCBI Accession # 4K2U_I (233aa) | |
| MALA28 | Light chain | anti-MSP1 | Sowa, K. M. et al., Isolation of a monoclonal | 23295 |
| | | MAD20 block2 | antibody from a malaria patient-derived phage | |
| | | ScFv Ig heavy | display library recognizing the Block 2 region | |
| | | chain variable | of Plasmodium falciparum merozoite surface | |
| | | region, partial | protein-1, Mol. Biochem. Parasitol. 112 (1), | |
| | | | 143-147 (2001), NCBI Accession | |
| | | | #AAK08697.1 (119aa) | |
| MALA29 | Light chain | anti-MSP1 | Sowa, K. M. et al., Isolation of a monoclonal | 23296 |
| | | MAD20 block2 | antibody from a malaria patient-derived phage | |
| | | ScFv Ig light | display library recognizing the Block 2 region | |
| | | chain variable | of Plasmodium falciparum merozoite surface | |
| | | region, partial | protein-1, Mol. Biochem. Parasitol. 112 (1), | |
| | | | 143-147 (2001), NCBI Accession | |
| | | | #AAK08698.1 (110aa) | |
| MALA30 | Light chain | immunoglobulin | Wajanarogana, S. et al., Construction of a | 23297 |
| | | light chain | human functional single-chain variable fragment | |
| | | variable region, | (scFv) antibody recognizing the malaria parasite | |
| | | partial | Plasmodium falciparum, Biotechnol. Appl. | |
| | | | Biochem. 44 (PT 1), 55-61 (2006) AAX76833.1 | |
| | | | (107aa) | |
| MALA31 | Kappa light chain | immunoglobulin | Lundquist, R. et al., Human recombinant | 23298 |
| | | kappa light | antibodies against Plasmodium falciparum | |
| | | chain variable | merozoite surface protein 3 cloned from | |
| | | region, partial | peripheral blood leukocytes of individuals with | |
| | | | immunity to malaria demonstrate antiparasitic | |
| | | | properties, Infect. Immun. 74 (6), 3222-3231, | |
| | | | (2006), NCBI Accession # AAT09787.1 | |
| | | | (113aa) | |
| MALA32 | Light chain | 2A10 anti- | NCBI Accession # BAK41503.1 (108aa) | 23299 |
| | variable region | malaria | | |
| | | antibody | | |
| MALA33 | Light chain | | U.S. Pat. No. 7,811,569 to Dziegiel; SEQ ID NO: 2 | 23300 |
| MALA34 | Light chain, Anti- | | U.S. Pat. No. 7,811,569 to Dziegiel; SEQ ID NO: 4 | 23301 |
| | ang-2 antibody | | | |
| MALA35 | Light chain | | U.S. Pat. No. 7,811,569 to Dziegiel; SEQ ID NO: 6 | 23302 |
| MALA36 | Light chain | | US20150197562 SEQ ID NO: 15 | 23303 |
| | variable region | | | |
| MALA37 | Light chain | | US20150197562 SEQ ID NO: 19 | 23304 |
| | variable region | | | |
| MALA38 | Light chain | mAb 5D5 | US20150158941 SEQ ID NO: 14 | 23305 |
| | variable region | | | |
| MALA39 | Light chain | | US20140112930 SEQ ID NO: 20 | 23306 |
| | variable region | | | |
| MALA40 | Light chain | M071Xi0199 | WO2014087007; SEQ ID NO: 184 | 23307 |
| | variable region | | | |
| MALA41 | Light chain | M071Xi2204 | WO2014087007; SEQ ID NO: 188 | 23308 |
| | variable region | | | |
| MALA42 | Light chain | M071Xi0237 | WO2014087007; SEQ ID NO: 192 | 23309 |
| | variable region | | | |
| MALA43 | Light chain | M071Xi2127 | WO2014087007; SEQ ID NO: 196 | 23310 |
| | variable region | | | |
| MALA44 | Light chain | M071Xi0092 | WO2014087007; SEQ ID NO: 200 | 23311 |
| | variable region | | | |
| MALA45 | Light chain | M071Xi2057 | WO2014087007; SEQ ID NO: 204 | 23312 |
| | variable region | | | |
| MALA46 | Light chain | M070Xi3010 | WO2014087007; SEQ ID NO: 208 | 23313 |
| | variable region | | | |
| MALA47 | Light chain | M071Xi0227 | WO2014087007; SEQ ID NO: 212 | 23314 |
| | variable region | | | |
| MALA48 | Light chain | M071Xi0081 | WO2014087007; SEQ ID NO: 216 | 23315 |
| | variable region | | | |
| MALA49 | Light chain | M071Xi0124 | WO2014087007; SEQ ID NO: 220 | 23316 |
| | variable region | | | |
| MALA50 | Light chain | M036Xi0326 | WO2014087007; SEQ ID NO: 224 | 23317 |
| | variable region | | | |
| MALA51 | Light chain | M070Xi3195 | WO2014087007; SEQ ID NO: 228 | 23318 |
| | variable region | | | |
| MALA52 | Light chain | M070Xi3062 | WO2014087007; SEQ ID NO: 232 | 23319 |
| | variable region | | | |
| MALA53 | Light chain | M071Xi2217 | WO2014087007; SEQ ID NO: 236 | 23320 |
| | variable region | | | |
| MALA54 | Light chain | M036Xi0003 | WO2014087007; SEQ ID NO: 240 | 23321 |
| | variable region | | | |
| MALA55 | Light chain, Eba- | R217 | Chen et al., PLoS Pathol. 9 (5), E1003390 | 23322 |
| | 175 | | (2013), NCBI Accession # 4QEX_M (214aa) | |
| MALA56 | Light chain, Eba- | R218 | Chen et al., PLoS Pathol. 9 (5), E1003390 | 23323 |
| | 175 | | (2013), NCBI Accession # 4K2U_M (234aa) | |
| MALA57 | Vivax apical | F8.12.19 | NCBI Accession # 2J4W_L (213aa) | 23324 |
| | membrane antigen | | | |
| | 1 monoclonal | | | |
| | antibody, seqres | | | |

In some embodiments, the payload region of the viral particle comprises one or more nucleic acid sequences encoding one or more of the payload antibody polypeptides listed in Table 30 against Ebola and/or Margburg Viruses (EBOL1-EBOL53; SEQ ID NO: 23325-23377),

TABLE 30
Antibodies against Ebola and Marburg viruses

| | | | | SEQ |
|---|---|---|---|---|
| Antibody | | Antibody | | ID |
| No. | Description | Name | Reference Information | NO |
| EBOL1 | Chain A, Sudan Ebolavirus | 16f6 | Bale et al., Structural basis for | 23325 |
| | Glycoprotein (Strain Boniface) | | differential neutralization of | |
| | | | ebolaviruses; Viruses 4 (4), 447-470 | |
| | | | (2012), NCBI Accession # 3VE0_B | |
| | | | (212aa) | |
| EBOL2 | Chain B, Sudan Ebolavirus | 16f6 | Bale et al., Structural basis for | 23326 |
| | Glycoprotein (Strain Boniface) | | differential neutralization of | |
| | | | ebolaviruses; Viruses 4 (4), 447-470 | |
| | | | (2012), NCBI Accession # 3VE0_A | |
| | | | (220aa) | |
| EBOL3 | Ebola Virus Glycoprotein | 13f6-1-2 | Lee J. E. et al., Complex of a protective | 23327 |
| | | Fab | antibody with its Ebola virus GP | |
| | | | peptide epitope: unusual features of a V | |
| | | | lambda x light chain; J. Mol. Biol. 375 | |
| | | | (1), 202-216 (2008), NCBI Accession # | |
| | | | 2QHR_L (218aa) | |
| EBOL4 | Ebola Virus Glycoprotein | 13f6-1-2 | Lee J. E. et al., Complex of a protective | 23328 |
| | | Fab | antibody with its Ebola virus GP | |
| | | | peptide epitope: unusual features of a V | |
| | | | lambda x light chain; J. Mol. Biol. 375 | |
| | | | (1), 202-216 (2008), NCBI Accession # | |
| | | | 2QHR_H (222aa) | |
| EBOL5 | Fab heavy chain Envelope | Mr78 | Hashiguchi, T., et al., Cell 160 (5), | 23329 |
| | Glycoprotein Gp1 | | 904-912 (2015), NCBI Accession # | |
| | | | 3X2D_P (226aa) | |
| EBOL6 | Fab light chain, Envelope | Mr78 | Hashiguchi, T., et al., Cell 160 (5), | 23330 |
| | Glycoprotein Gp1 | | 904-912 (2015), NCBI Accession # | |
| | | | 3X2D_O (213aa) | |
| EBOL7 | Fusion protein, Zaire Ebola virus, | | US20140356354 SEQ ID NO: 2 | 23331 |
| | Mayinga strain glycoprotein | | | |
| EBOL8 | Heavy chain Ebolavirus-Protective | | Olal, D., et al., Structure of an | 23332 |
| | Antibody | | Antibody in Complex with Its Mucin | |
| | | | Domain Linear Epitope That Is | |
| | | | Protective against Ebola Virus; J. Virol. | |
| | | | 86 (5), 2809-2816 (2012), NCBI | |
| | | | Accession # 2Y6S_H (213aa) | |
| EBOL9 | Heavy chain Filovirus (Ebola or | | US20140356354 SEQ ID NO: 6 | 23333 |
| | Marburg) | | | |
| EBOL10 | Heavy chain Filovirus (Ebola or | | US20140356354 SEQ ID NO: 7 | 23334 |
| | Marburg) | | | |
| EBOL11 | Heavy chain Filovirus (Ebola or | | US20140356354 SEQ ID NO: 8 | 23335 |
| | Marburg) | | | |
| EBOL12 | Heavy chain Filovirus (Ebola or | | US20140356354 SEQ ID NO: 9 | 23336 |
| | Marburg) | | | |
| EBOL13 | Heavy chain Filovirus (Ebola or | | US20140356354 SEQ ID NO: 10 | 23337 |
| | Marburg) | | | |
| EBOL14 | Heavy chain Filovirus (Ebola or | | US20140356354 SEQ ID NO: 11 | 23338 |
| | Marburg) | | | |
| EBOL15 | Heavy chain variable region, Zaire | | WO2015127136 SEQ ID NO: 71 | 23339 |
| | ebolavirus (ZEBOV) glycoprotein | | | |
| EBOL16 | Heavy chain variable region, Zaire | | WO2015127136 SEQ ID NO: 47 | 23340 |
| | ebolavirus (ZEBOV) glycoprotein | | | |
| EBOL17 | Heavy chain variable region, Zaire | | WO2015127136 SEQ ID NO: 23 | 23341 |
| | ebolavirus (ZEBOV) glycoprotein | | | |
| EBOL18 | Heavy chain variable region, Ebola | 16H11 | U.S. Pat. No. 9,097,713 SEQ ID NO: 2 | 23342 |
| | Sudan Boniface virus (ESB) | | | |
| | glycoprotein (GP) | | | |
| EBOL19 | Heavy chain variable region, Ebola | 19B3 | U.S. Pat. No. 9,097,713 SEQ ID NO: 4 | 23343 |
| | Sudan Boniface virus (ESB) | | | |
| | glycoprotein (GP) | | | |
| EBOL20 | Heavy chain variable region, Ebola | 17F6 | U.S. Pat. No. 9,097,713 SEQ ID NO: 6 | 23344 |
| | Sudan Boniface virus (ESB) | | | |
| | glycoprotein (GP) | | | |
| EBOL21 | Heavy chain variable region, Ebola | 16F6 | U.S. Pat. No. 9,097,713 SEQ ID NO: 8 | 23345 |
| | Sudan Boniface virus (ESB) | | | |
| | glycoprotein (GP) | | | |
| EBOL22 | Heavy chain variable region, Ebola | EGP 6D8 | U.S. Pat. No. 7,335,356 SEQ ID NO: 22 | 23346 |
| | virus GP | 1-2 | | |
| EBOL23 | Heavy chain variable region, Ebola | EGP13F6-1-2 | U.S. Pat. No. 7,335,356 SEQ ID NO: 32 | 23347 |
| | virus GP | | | |
| EBOL24 | Heavy chain variable region, Ebola | EGP13C6-1-1 | U.S. Pat. No. 7,335,356 SEQ ID NO: 12 | 23348 |
| | virus GP | | | |
| EBOL25 | Heavy chain variable region, | | WO2015127140 SEQ ID NO: 14 | 23349 |
| | Marburg virus, Ebola virus, Sudan | | | |
| | virus, Bundibugyo virus, Tai | | | |
| | Forest virus or Reston virus | | | |
| | glycoprotein | | | |
| EBOL26 | Heavy chain variable region, | | WO2015127140 SEQ ID NO: 38 | 23350 |
| | Marburg virus, Ebola virus, Sudan | | | |
| | virus, Bundibugyo virus, Tai | | | |
| | Forest virus or Reston virus | | | |
| | glycoprotein | | | |
| EBOL27 | Heavy chain variable region, | | WO2015127140 SEQ ID NO: 62 | 23351 |
| | Marburg virus, Ebola virus, Sudan | | | |
| | virus, Bundibugyo virus, Tai | | | |
| | Forest virus or Reston virus | | | |
| | glycoprotein | | | |
| EBOL28 | Heavy chain variable region, | | WO2015127140 SEQ ID NO: 86 | 23352 |
| | Marburg virus, Ebola virus, Sudan | | | |
| | virus, Bundibugyo virus, Tai | | | |
| | Forest virus or Reston virus | | | |
| | glycoprotein | | | |
| EBOL29 | Heavy chain variable region, | | WO2015127140 SEQ ID NO: 110 | 23353 |
| | Marburg virus, Ebola virus, Sudan | | | |
| | virus, Bundibugyo virus, Tai | | | |
| | Forest virus or Reston virus | | | |
| | glycoprotein | | | |
| EBOL30 | Heavy chain variable region, | | WO2015127140 SEQ ID NO: 134 | 23354 |
| | Marburg virus, Ebola virus, Sudan | | | |
| | virus, Bundibugyo virus, Tai | | | |
| | Forest virus or Reston virus | | | |
| | glycoprotein | | | |
| EBOL31 | Heavy chain variable region, | | WO2015127140 SEQ ID NO: 158 | 23355 |
| | Marburg virus, Ebola virus, Sudan | | | |
| | virus, Bundibugyo virus, Tai | | | |
| | Forest virus or Reston virus | | | |
| | glycoprotein | | | |
| EBOL32 | Heavy chain, Ebola virus | Fab Kz52 | Lee J. E. et al., Structure of the Ebola | 23356 |
| | glycoprotein, | | virus glycoprotein bound to an | |
| | | | antibody from a human survivor; | |
| | | | Nature 454 (7201), 177-182 (2008), | |
| | | | NCBI Accession # 3CSY_G (226aa) | |
| EBOL33 | Light chain variable region, Ebola | 16F6 | U.S. Pat. No. 9,097,713 SEQ ID NO: 10 | 23357 |
| | Sudan Boniface virus (ESB) | | | |
| | glycoprotein (GP) | | | |
| EBOL34 | Light chain variable region, Ebola | EGP 6D8 | U.S. Pat. No. 7,335,356 SEQ ID NO: 27 | 23358 |
| | virus GP | 1-2 | | |
| EBOL35 | Light chain variable region, Ebola | EGP13F6-1-2 | U.S. Pat. No. 7,335,356 SEQ ID NO: 37 | 23359 |
| | virus GP | | | |
| EBOL36 | Light chain variable region, Ebola | EGP13C6-1-1 | U.S. Pat. No. 7,335,356 SEQ ID NO: 16 | 23360 |
| | virus GP | | | |
| EBOL37 | Light chain variable region, | | WO2015127140 SEQ ID NO: 2 | 23361 |
| | Marburg virus, Ebola virus, Sudan | | | |
| | virus, Bundibugyo virus, Tai | | | |
| | Forest virus or Reston virus | | | |
| | glycoprotein | | | |
| EBOL38 | Light chain variable region, | | WO2015127140 SEQ ID NO: 26 | 23362 |
| | Marburg virus, Ebola virus, Sudan | | | |
| | virus, Bundibugyo virus, Tai | | | |
| | Forest virus or Reston virus | | | |
| | glycoprotein | | | |
| EBOL39 | Light chain variable region, | | WO2015127140 SEQ ID NO: 50 | 23363 |
| | Marburg virus, Ebola virus, Sudan | | | |
| | virus, Bundibugyo virus, Tai | | | |
| | Forest vires or Reston virus | | | |
| | glycoprotein | | | |
| EBOL40 | Light chain variable region, | | WO2015127140 SEQ ID NO: 74 | 23364 |
| | Marburg virus, Ebola virus, Sudan | | | |
| | virus, Bundibugyo virus, Tai | | | |
| | Forest virus or Reston virus | | | |
| | glycoprotein | | | |
| EBOL41 | Light chain variable region, | | WO2015127140 SEQ ID NO: 98 | 23365 |
| | Marburg virus, Ebola virus, Sudan | | | |
| | virus, Bundibugyo virus, Tai | | | |
| | Forest virus or Reston virus | | | |
| | glycoprotein | | | |
| EBOL42 | Light chain variable region, | | WO2015127140 SEQ ID NO: 122 | 23366 |
| | Marburg virus, Ebola virus, Sudan | | | |
| | virus, Bundibugyo virus, Tai | | | |
| | Forest virus or Reston virus | | | |
| | glycoprotein | | | |
| EBOL43 | Light chain variable region, | | WO2015127140 SEQ ID NO: 146 | 23367 |
| | Marburg virus, Ebola virus, Sudan | | | |
| | virus, Bundibugyo virus, Tai | | | |
| | Forest virus or Reston virus | | | |
| | glycoprotein | | | |
| EBOL44 | Light chain variable region, Zaire | | WO2015127136 SEQ ID NO: 59 | 23368 |
| | ebolavirus (ZEBOV) glycoprotein | | | |
| EBOL45 | Light chain variable region, Zaire | | WO2015127136 SEQ ID NO: 35 | 23369 |
| | ebolavirus (ZEBOV) glycoprotein | | | |
| EBOL46 | Light chain variable region, Zaire | | WO2015127136 SEQ ID NO: 11 | 23370 |
| | ebolavirus (ZEBOV) glycoprotein | | | |
| EBOL47 | light chain, Ebola virus | Fab Kz52 | Lee J. E. et al., Structure of the Ebola | 23371 |
| | glycoprotein | | virus glycoprotein bound to an | |
| | | | antibody from a human survivor; | |
| | | | Nature 454 (7201), 177-182 (2008), | |
| | | | NCBI Accession # 3CSY_H (217aa) | |
| EBOL48 | Light chain, Ebolavirus-Protective | | Olal, D., et al., Structure of an | 23372 |
| | Antibody | | Antibody in Complex with Its Mucin | |
| | | | Domain Linear Epitope That Is | |
| | | | Protective against Ebola Virus; J. Virol. | |
| | | | 86 (5), 2809-2816 (2012), NCBI | 23373 |
| | | | Accession # 2Y6S_L (217aa) | |
| EBOL49 | Light chain, Filovirus (Ebola or | | US20140356354 SEQ ID NO: 12 | 23374 |
| | Marburg) | | | |
| EBOL50 | Light chain, Filovirus (Ebola or | | US20140356354 SEQ ID NO: 13 | 23375 |
| | Marburg) | | | |
| EBOL51 | Light chain, Filovirus (Ebola or | | US20140356354 SEQ ID NO: 14 | 23376 |
| | Marburg) | | | |
| EBOL52 | Light chain, Filovirus (Ebola or | | US20140356354 SEQ ID NO: 15 | 23377 |
| | Marburg) | | | |
| EBOL53 | Light chain, Filovirus (Ebola or | | US20140356354 SEQ ID NO: 16 | 23378 |
| | Marburg) | | | |

In some embodiments, the payload region of the viral particle comprises one or more nucleic acid sequences, fragment or variants thereof or encodes one or more polypeptides, fragments or variants thereof described in U.S. Pat. No. 7,335,356 and EP Pub. No. EP1539238, the contents of each of which are herein incorporated by reference in their entirety, against Ebola.

In some embodiments, the payload region of the viral particle comprises one or more nucleic acid sequences encoding one or more of the payload antibody polypeptides listed in Table 31 against Mosquito-borne disease (MOSQ1-MOSQ118; SEQ ID NO: 23378-23495).

TABLE 31
Antibodies against Mosquito-borne diseases

| | | | | SEQ |
|---|---|---|---|---|
| Antibody | | Antibody | | ID |
| No. | Description | Name | Reference Information | NO |
| MOSQ1 | Gamma heavy chain, | | Thibodeaux, B. A. “Development of a human- | 23378 |
| | partial, anti-Saint Louis | | murine chimeric immunoglobulin M antibody | |
| | encephalitis virus | | for use in the serological detection of human | |
| | envelope glycoprotein | | flavivirus antibodies”, Clin. Vaccine | |
| | immunoglobulin | | Immunol. 16 (5), 679-685, 2009), NCBI | |
| | | | Accession # ACI62179 | |
| MOSQ2 | Gamma heavy chain, | | Thibodeaux, B. A. “Development of a human- | 23379 |
| | partial, anti-Saint Louis | | murine chimeric immunoglobulin M antibody | |
| | encephalitis virus | | for use in the serological detection of human | |
| | envelope glycoprotein | | flavivirus antibodies”, Clin. Vaccine | |
| | immunoglobulin | | Immunol. 16 (5), 679-685, 2009), NCBI | |
| | | | Accession # ACI62180 | |
| MOSQ3 | Heavy chain variable | anti- | US20080292644 SEQ ID NO: 69 | 23380 |
| | region, Japanese | DLVR1/CLEC5A | | |
| | encephalitis virus | | | |
| MOSQ4 | Heavy chain variable | anti- | US20080292644 SEQ ID NO: 70 | 23381 |
| | region, Japanese | DLVR1/CLEC5A | | |
| | encephalitis virus | | | |
| MOSQ5 | Heavy chain variable | anti- | US20080292644 SEQ ID NO: 71 | 23382 |
| | region, Japanese | DLVR1/CLEC5A | | |
| | encephalitis virus | | | |
| MOSQ6 | Heavy chain variable | | CN103864925 SEQ ID NO: 2 | 23383 |
| | region, Japanese | | | |
| | encephalitis virus | | | |
| MOSQ7 | Heavy chain variable | | Throsby, M. “Isolation and characterization | 23384 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20480.1 | |
| MOSQ8 | Heavy chain variable | | Throsby, M. “Isolation and characterization | 23385 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20479.1 | |
| MOSQ9 | Heavy chain variable | | Throsby, M. “Isolation and characterization | 23386 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20478.1 | |
| MOSQ10 | Heavy chain variable | | Throsby, M. “Isolation and characterization | 23387 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20477.1 | |
| MOSQ11 | Heavy chain variable | | Throsby, M. “Isolation and characterization | 23388 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20476.1 | |
| MOSQ12 | Heavy chain variable | | Throsby, M. “Isolation and characterization | 23389 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20475.1 | |
| MOSQ13 | Heavy chain variable | | Throsby, M. “Isolation and characterization | 23390 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20474.1 | |
| MOSQ14 | Heavy chain variable | | Throsby, M. “Isolation and characterization | 23391 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20473.1 | |
| MOSQ15 | Heavy chain variable | | Throsby, M. “Isolation and characterization | 23392 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20472.1 | |
| MOSQ16 | Heavy chain variable | | Throsby, M. “Isolation and characterization | 23393 |
| | region, partial sequence, | | of human monoclonal antibodies from | |
| | WNV | | individuals infected with west nile virus” J. | |
| | | | Virol. 80 (14), 6982-6992 (2006). NCBI | |
| | | | Accession # ABF20471.1 | |
| MOSQ17 | Heavy chain variable | mAbl 1 | WO2014144061 SEQ ID NO: 1 | 23394 |
| | region, WNV, Dengue, | | | |
| | St. Louis encephalitis, | | | |
| | yellow fever virus, | | | |
| | Japanese encephalitis | | | |
| | virus, Murray Valley | | | |
| | encephalitis virus | | | |
| MOSQ18 | Heavy chain, WNV | CR4348 | U.S. Pat. No. 8,911,738 SEQ ID NO: 30 | 23395 |
| MOSQ19 | Heavy chain, WNV | CR4354 | U.S. Pat. No. 8,911,738 SEQ ID NO: 32 | 23396 |
| MOSQ20 | Heavy chain, WNV | CR4261 | U.S. Pat. No. 8,911,738 SEQ ID NO: 60 | 23397 |
| MOSQ21 | Heavy chain, WNV | CR4267 | U.S. Pat. No. 8,911,738 SEQ ID NO: 62 | 23398 |
| MOSQ22 | Heavy chain, WNV | CR4328 | U.S. Pat. No. 8,911,738 SEQ ID NO: 64 | 23399 |
| MOSQ23 | Heavy chain, WNV | CR4335 | U.S. Pat. No. 8,911,738 SEQ ID NO: 66 | 23400 |
| MOSQ24 | Heavy chain, WNV | CR4383 | U.S. Pat. No. 8,911,738 SEQ ID NO: 68 | 23401 |
| MOSQ25 | Heavy chain, WNV | CRM4354 | U.S. Pat. No. 8,911,738 SEQ ID NO: 148 | 23402 |
| MOSQ26 | Heavy chain variable | Antibody from | U.S. Pat. No. 8,911,738 SEQ ID NO: 20 | 23403 |
| | region, WNV | U.S. Pat. No. 8,911,738 | | |
| MOSQ27 | Heavy chain variable | E16 heavy chain | U.S. Pat. No. 7,572,456 SEQ ID NO: 21 | 23404 |
| | region, WNV | version 1 | | |
| MOSQ28 | Heavy chain variable | E16 heavy chain | U.S. Pat. No. 7,572,456 SEQ ID NO: 22 | 23405 |
| | region, WNV | version 2 | | |
| MOSQ29 | Heavy chain variable | E16 heavy chain | U.S. Pat. No. 7,572,456 SEQ ID NO: 23 | 23406 |
| | region, WNV | version 3 | | |
| MOSQ30 | Heavy chain variable | Antibody from | U.S. Pat. No. 8,911,738 SEQ ID NO: 18 | 23407 |
| | region, WNV | U.S. Pat. No. 8,911,738 | | |
| MOSQ31 | Heavy chain variable | hu-E16/E16p | U.S. Pat. No. 8,663,950 SEQ ID NO: 2 | 23408 |
| | region, WNV | | | |
| MOSQ32 | Heavy chain variable | hu-E16/E16p | U.S. Pat. No. 8,663,950 SEQ ID NO: 3 | 23409 |
| | region, WNV | | | |
| MOSQ33 | Heavy chain variable | E16 | U.S. Pat. No. 7,527,973 SEQ ID NO: 4 | 23410 |
| | region, WNV | | | |
| MOSQ34 | Heavy chain variable | E24 | U.S. Pat. No. 7,527,973 SEQ ID NO: 8 | 23411 |
| | region, WNV | | | |
| MOSQ35 | Heavy chain variable | E34 | U.S. Pat. No. 7,527,973 SEQ ID NO: 12 | 23412 |
| | region, WNV | | | |
| MOSQ36 | Heavy chain variable | 11 | US20090130123 SEQ ID NO: 23 | 23413 |
| | region, WNV | | | |
| MOSQ37 | Heavy chain variable | 71 | US20090130123 SEQ ID NO: 24 | 23414 |
| | region, WNV | | | |
| MOSQ38 | Heavy chain variable | 73 | US20090130123 SEQ ID NO: 25 | 23415 |
| | region, WNV | | | |
| MOSQ39 | Heavy chain variable | 85 | US20090130123 SEQ ID NO: 26 | 23416 |
| | region, WNV | | | |
| MOSQ40 | Heavy chain variable | 15 | US20090130123 SEQ ID NO: 27 | 23417 |
| | region, WNV | | | |
| MOSQ41 | Heavy chain variable | 95 | US20090130123 SEQ ID NO: 28 | 23418 |
| | region, WNV | | | |
| MOSQ42 | Heavy chain variable | 84 | US20090130123 SEQ ID NO: 29 | 23419 |
| | region, WNV | | | |
| MOSQ43 | Heavy chain variable | 10 | US20090130123 SEQ ID NO: 30 | 23420 |
| | region, WNV | | | |
| MOSQ44 | Heavy chain variable | 69 | US20090130123 SEQ ID NO: 31 | 23421 |
| | region, WNV | | | |
| MOSQ45 | Heavy chain variable | 79 | US20090130123 SEQ ID NO: 32 | 23422 |
| | region, WNV | | | |
| MOSQ46 | Heavy chain variable | 94 | US20090130123 SEQ ID NO: 33 | 23423 |
| | region, WNV | | | |
| MOSQ47 | Heavy chain variable | 9FI2 | WO2010093335 SEQ ID NO: 4 | 23424 |
| | region, WNV | | | |
| MOSQ48 | Heavy chain variable | | Throsby, M. “Isolation and characterization | 23425 |
| | region, partial sequence, | | of human monoclonal antibodies from | |
| | WMV | | individuals infected with west nile virus” J. | |
| | | | Virol. 80 (14), 6982-6992 (2006), NCBI | |
| | | | Accession # ABF20481.1 | |
| MOSQ49 | Heavy chain translation, | hu-E16/E16p | U.S. Pat. No. 8,663,950 SEQ ID NO: 5 | 23426 |
| | WNV | | | |
| MOSQ50 | Heavy chain variable | anti-yellow fever | Thibodeaux, B. A. “A humanized IgG but not | 23427 |
| | region, Yellow fever | virus vaccine | IgM antibody is effective in prophylaxis and | |
| | virus | strain 17D E | therapy of yellow fever infection in an | |
| | | glycoprotein | AG129/17D-204 peripheral challenge mouse | |
| | | | model” Antiviral Res. 94 (1), 1-8 (2012), | |
| | | | NCBI Accession # ADO17683 | |
| MOSQ51 | Light chain variable | anti- | US20080292644 SEQ ID NO: 66 | 23428 |
| | region, Japanese | DLVR1/CLEC5A | | |
| | encephalitis virus | | | |
| MOSQ52 | Light chain variable | anti- | US20080292644 SEQ ID NO: 67 | 23429 |
| | region, Japanese | DLVR1/CLEC5A | | |
| | encephalitis virus | | | |
| MOSQ53 | Light chain variable | anti- | US20080292644 SEQ ID NO: 68 | 23430 |
| | region, Japanese | DLVR1/CLEC5A | | |
| | encephalitis virus | | | |
| MOSQ54 | Light chain variable | | CN103864925 SEQ ID NO: 1 | 23431 |
| | region, Japanese | | | |
| | encephalitis virus | | | |
| MOSQ55 | Light chain variable | mAbl 1 | WO2014144061 SEQ ID NO: 3 | 23432 |
| | region, WNV, Dengue, | | | |
| | St. Louis encephalitis, | | | |
| | yellow fever virus, | | | |
| | Japanese encephalitis | | | |
| | virus, Murray Valley | | | |
| | encephalitis virus | | | |
| MOSQ56 | Light chain, WNV | CR4348 | U.S. Pat. No. 8,911,738 SEQ ID NO: 34 | 23433 |
| MOSQ57 | Light chain, WNV | CR4354 | U.S. Pat. No. 8,911,738 SEQ ID NO: 36 | 23434 |
| MOSQ58 | Light chain, WNV | CR4261 | U.S. Pat. No. 8,911,738 SEQ ID NO: 70 | 23435 |
| MOSQ59 | Light chain, WNV | CR4267 | U.S. Pat. No. 8,911,738 SEQ ID NO: 72 | 23436 |
| MOSQ60 | Light chain, WNV | CR4328 | U.S. Pat. No. 8,911,738 SEQ ID NO: 74 | 23437 |
| MOSQ61 | Light chain, WNV | CR4335 | U.S. Pat. No. 8,911,738 SEQ ID NO: 76 | 23438 |
| MOSQ62 | Light chain, WNV | CR4383 | U.S. Pat. No. 8,911,738 SEQ ID NO: 78 | 23439 |
| MOSQ63 | Light chain variable | Antibody from | U.S. Pat. No. 8,911,738 SEQ ID NO: 22 | 23440 |
| | region, WNV | U.S. Pat. No. 8,911,738 | | |
| MOSQ64 | Light chain variable | Antibody from | U.S. Pat. No. 8,911,738 SEQ ID NO: 24 | 23441 |
| | region, WNV | U.S. Pat. No. 8,911,738 | | |
| MOSQ65 | Light chain variable | E16 | U.S. Pat. No. 7,527,973 SEQ ID NO: 2 | 23442 |
| | region, WNV | | | |
| MOSQ66 | Light chain variable | E24 | U.S. Pat. No. 7,527,973 SEQ ID NO: 6 | 23443 |
| | region, WNV | | | |
| MOSQ67 | Light chain variable | E34 | U.S. Pat. No. 7,527,973 SEQ ID NO: 10 | 23444 |
| | region, WNV | | | |
| MOSQ68 | Light chain variable | E16 light chain | U.S. Pat. No. 7,572,456 SEQ ID NO: 25 | 23445 |
| | region, WNV | version 1 | | |
| MOSQ69 | Light chain variable | E16 light chain | U.S. Pat. No. 7,572,456 SEQ ID NO: 26 | 23446 |
| | region, WNV | version 2 | | |
| MOSQ70 | Light chain variable | 11 | US20090130123 SEQ ID NO: 34 | 23447 |
| | region, WNV | | | |
| MOSQ71 | Light chain variable | 71 | US20090130123 SEQ ID NO: 35 | 23448 |
| | region, WNV | | | |
| MOSQ72 | Light chain variable | 73 | US20090130123 SEQ ID NO: 36 | 23449 |
| | region, WNV | | | |
| MOSQ73 | Light chain variable | 85 | US20090130123 SEQ ID NO: 37 | 23450 |
| | region, WNV | | | |
| MOSQ74 | Light chain variable | 15 | US20090130123 SEQ ID NO: 38 | 23451 |
| | region, WNV | | | |
| MOSQ75 | Light chain variable | 95 | US20090130123 SEQ ID NO: 39 | 23452 |
| | region, WNV | | | |
| MOSQ76 | Light chain variable | 84 | US20090130123 SEQ ID NO: 40 | 23453 |
| | region, WNV | | | |
| MOSQ77 | Light chain variable | 10 | US20090130123 SEQ ID NO: 41 | 23454 |
| | region, WNV | | | |
| MOSQ78 | Light chain variable | | US20090130123 SEQ ID NO: 42 | 23455 |
| | region, WNV | | | |
| MOSQ79 | Light chain variable | 79 | US20090130123 SEQ ID NO: 43 | 23456 |
| | region, WNV | | | |
| MOSQ80 | Light chain variable | 94 | US20090130123 SEQ ID NO: 44 | 23457 |
| | region, WNV | | | |
| MOSQ81 | Light chain variable | 9FI2 | WO2010093335 SEQ ID NO: 6 | 23458 |
| | region, WNV | | | |
| MOSQ82 | Light chain variable | | Throsby, M. “Isolation and characterization | 23459 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20470.1 | |
| MOSQ83 | Light chain variable | | Throsby, M. “Isolation and characterization | 23460 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20469.1 | |
| MOSQ84 | Light chain variable | | Throsby, M. “Isolation and characterization | 23461 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20468.1 | |
| MOSQ85 | Light chain variable | | Throsby, M. “Isolation and characterization | 23462 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20467.1 | |
| MOSQ86 | Light chain variable | | Throsby, M. “Isolation and characterization | 23463 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20466.1 | |
| MOSQ87 | Light chain variable | | Throsby, M. “Isolation and characterization | 23464 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20465.1 | |
| MOSQ88 | Light chain variable | | Throsby, M. “Isolation and characterization | 23465 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20464.1 | |
| MOSQ89 | Light chain variable | | Throsby, M. “Isolation and characterization | 23466 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20463.1 | |
| MOSQ90 | Light chain variable | | Throsby, M. “Isolation and characterization | 23467 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20462.1 | |
| MOSQ91 | Light chain variable | | Throsby, M. “Isolation and characterization | 23468 |
| | region, partial sequence, | | of human monoclonal antibodies from | |
| | WNV | | individuals infected with west nile virus” J, | |
| | | | Virol. 80 (14), 6982-6992 (2006), NCBI | |
| | | | Accession # ABF20461.1 | |
| MOSQ92 | Light chain variable | | Throsby, M. “Isolation and characterization | 23469 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20460.1 | |
| MOSQ93 | Light chain variable | | Throsby, M. “Isolation and characterization | 23470 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20459.1 | |
| MOSQ94 | Light chain variable | | Throsby, M. “Isolation and characterization | 23471 |
| | region, partial sequence, | | of human monoclonal antibodies from | |
| | WNV | | individuals infected with west nile virus” J. | |
| | | | Virol. 80 (14), 6982-6992 (2006), NCBI | |
| | | | Accession # ABF20458.1 | |
| MOSQ95 | Light chain translation, | hu-E16/E16p | U.S. Pat. No. 8,663,950 SEQ ID NO: 7 | 23472 |
| | WNV | | | |
| MOSQ96 | Light chain variable | anti-yellow fever | Thibodeaux, B. A. “A humanized IgG but not | 23473 |
| | region, Yellow fever | virus vaccine | IgM antibody is effective in prophylaxis and | |
| | virus | strain 17D E | therapy of yellow fever infection in an | |
| | | glycoprotein | AG129/17D-204 peripheral challenge mouse | |
| | | | model” Antiviral Res. 94 (1), 1-8 (2012), | |
| | | | NCBI Accession # ADO17684 | |
| MOSQ97 | ScFv, WNV | 9FI2 | WO2010093335 SEQ ID NO: 8 | 23474 |
| MOSQ98 | Fc region, WNV, | mAb-11 | WO2014144061 SEQ ID NO: 5 | 23475 |
| | Dengue, St. Louis | | | |
| | encephalitis, yellow | | | |
| | fever virus, Japanese | | | |
| | encephalitis virus, | | | |
| | Murray Valley | | | |
| | encephalitis virus | | | |
| MOSQ99 | Fc region, WNV, | mAb-11-LALA | WO2014144061 SEQ ID NO: 6 | 23476 |
| | Dengue, St. Louis | | | |
| | encephalitis, yellow | | | |
| | fever virus, Japanese | | | |
| | encephalitis virus, | | | |
| | Murray Valley | | | |
| | encephalitis virus | | | |
| MOSQ100 | ScFv, WNV | 11 | US20090130123 SEQ ID NO: 12 | 23477 |
| MOSQ101 | ScFv, WNV | 71 | US20090130123 SEQ ID NO: 13 | 23478 |
| MOSQ102 | ScFv, WNV | 73 | US20090130123 SEQ ID NO: 14 | 23479 |
| MOSQ103 | ScFv, WNV | 85 | US20090130123 SEQ ID NO: 15 | 23480 |
| MOSQ104 | ScFv, WNV | 15 | US20090130123 SEQ ID NO: 16 | 23481 |
| MOSQ105 | ScFv, WNV | 95 | US20090130123 SEQ ID NO: 17 | 23482 |
| MOSQ106 | ScFv, WNV | 84 | US20090130123 SEQ ID NO: 18 | 23483 |
| MOSQ107 | ScFv, WNV | 10 | US20090130123 SEQ ID NO: 19 | 23484 |
| MOSQ108 | ScFv, WNV | 69 | US20090130123 SEQ ID NO: 20 | 23485 |
| MOSQ109 | ScFv, WNV | 79 | US20090130123 SEQ ID NO: 21 | 23486 |
| MOSQ110 | ScFv, WNV | 94 | US20090130123 SEQ ID NO: 22 | 23487 |
| MOSQ111 | ScFvs, WNV | SC04-348 | U.S. Pat. No. 8,911,738 SEQ ID NO: 26 | 23488 |
| MOSQ112 | ScFvs, WNV | SC04-354 | U.S. Pat. No. 8,911,738 SEQ ID NO: 28 | 23489 |
| MOSQ113 | ScFv, Yellow | anti-yellow | Daffis, S. et al. “Antibody responses against | 23490 |
| | fever virus | fever virus E | wild-type yellow fever virus and the 17D | |
| | | protein scFv 7A | vaccine strain: characterization with human | |
| | | | monoclonal antibody fragments and | |
| | | | neutralization escape variants” Virology 337 | 23491 |
| | | | (2), 262-272 (2005), NCBI Accession # | |
| | | | AAT76799 | |
| MOSQ114 | ScFv, Yellow | anti-yellow | Daffis, S. et al. “Antibody responses against | 23492 |
| | fever virus | fever virus E | wild-type yellow fever virus and the 17D | |
| | | protein scFv | vaccine strain: characterization with human | |
| | | R3(27) | monoclonal antibody fragments and | |
| | | | neutralization escape variants” Virology 337 | |
| | | | (2), 262-272 (2005), NCBI Accession # | |
| | | | AAT76800 | |
| MOSQ115 | ScFv, Yellow | anti-yellow | Daffis, S. et al. “Antibody responses against | 23493 |
| | fever virus | fever virus E | wild-type yellow fever virus and the 17D | |
| | | protein scFv 5A | vaccine strain: characterization with human | |
| | | | monoclonal antibody fragments and | |
| | | | neutralization escape variants” Virology 337 | |
| | | | (2), 262-272 (2005), NCBI Accession # | |
| | | | AAT76801 | |
| MOSQ116 | ScFv, Yellow | anti-yellow | Daffis, S. et al. “Antibody responses against | 23494 |
| | fever virus | fever virus E | wild-type yellow fever virus and the 17D | |
| | | protein scFv 1A | vaccine strain: characterization with human | |
| | | | monoclonal antibody fragments and | |
| | | | neutralization escape variants” Virology 337 | |
| | | | (2), 262-272 (2005), NCBI Accession # | |
| | | | AAT76802 | |
| MOSQ117 | ScFv, Yellow | anti-yellow | Daffis, S. et al. “Antibody responses against | 23495 |
| | fever virus | fever virus E | wild-type yellow fever virus and the 17D | |
| | | protein scFv 2A | vaccine strain: characterization with human | |
| | | | monoclonal antibody fragments and | |
| | | | neutralization escape variants” Virology 337 | |
| | | | (2), 262-272 (2005), NCBI Accession # | |
| | | | AAT76803 | |
| MOSQ118 | ScFv, Yellow | anti-yellow | Daffis, S. et al. “Antibody responses against | 23496 |
| | fever virus | fever virus E | wild-type yellow fever virus and the 17D | |
| | | protein scFv | vaccine strain: characterization with human | |
| | | R3(9) | monoclonal antibody fragments and | |
| | | | neutralization escape variants” Virology 337 | |
| | | | (2), 262-272 (2005), NCBI Accession # | |
| | | | AAT76804 | |

In some embodiments, the payload region or me viral particle comprises one or more nucleic acid sequences, fragment or variants thereof or encodes one or more polypeptides, fragments or variants thereof described in U.S. Pat. No. 6,399,062 and US Pub. No. US20110171225, the contents of each of which are herein incorporated by reference in their entirety, against Malaria.

### Infectious Diseases

In some embodiments, the payload region of the viral particle comprises one or more nucleic acid sequences encoding one or more of the infectious disease related payload antibody polypeptides listed in Tables 32-53.

In some embodiments, the payload region of the viral particle comprises one or more nucleic acid sequences encoding one or more of the payload antibody polypeptides listed in Table 32 against Influenza virus (INFL1-INFL1085; SEQ ID NO: 23496-24580).

TABLE 32
Antibodies against Influenza virus

| | | | | SEQ |
|---|---|---|---|---|
| Antibody | | Antibody | | ID |
| No. | Description | Name | Reference Information | NO |
| INFL1 | Fab Fragment Heavy | ch65 | Whittle, J. R. et al., Broadly neutralizing | 23496 |
| | chain | | human antibody that recognizes the | |
| | | | receptor-binding pocket of influenza | |
| | | | virus hemagglutinin; Proc. Natl. Acad. | |
| | | | Sci. U.S.A. 108 (34), 14216-14221 | |
| | | | (2011), NCBI Accession #3SMS_H | |
| INFL2 | Fab Heavy Chain | Fab Cr6261 | Lingwood, D., et al., Structural and | 23497 |
| | | (Somatic Heavy | genetic basis for development of broadly | |
| | | Chain With | neutralizing influenza antibodies; Nature | |
| | | Germline- | 489 (7417), 566-570 (2012), NCBI | |
| | | Reverted Light | Accession #4EVN_M (242aa) | |
| | | Chain) | | |
| INFL3 | Fab heavy chain | Del2d1 | Krause, J. C. et al., M Bio 2 (1), E00345- | 23498 |
| | | | E00310 (2011), NCBI Accession | |
| | | | #3QHF_H | |
| INFL4 | Fab heavy chain | Fld194 Fab | Xiong, X. et al., Structures of complexes | 23499 |
| | | | formed by H5 influenza hemagglutinin | |
| | | | with a potent broadly neutralizing human | |
| | | | monoclonal antibody; Proc. Natl. Acad. | |
| | | | Sci. U.S.A. 112 (30), 9430-9435 (2015), | |
| | | | NCBI Accession #5A3I_C (230aa) | |
| INFL5 | Fab heavy chain | H5.3 | Winarski, K. L., Thornburg, N. J. et al., | 23500 |
| | | | Vaccine-elicited antibody that neutralizes | |
| | | | H5N1 influenza and variants binds the | |
| | | | receptor site and polymorphic sites | |
| | | | “PNAS 2015 112 (30) 9346-9351”, NCBI | |
| | | | Accession #4XNM_H | |
| INFL6 | Fab Heavy chain | 5j8 | Hong, M. et al., Antibody Recognition of | 23501 |
| | | | the Pandemic H1N1 Influenza Virus | |
| | | | Hemagglutinin Receptor Binding Site; J. | |
| | | | Virol. 87 (22), 12471-12480 (2013), | |
| | | | NCBI Accession #4M5Z_H | |
| INFL7 | Fab lambda heavy | CR6261 | Ekiert, D. C. et al., Antibody recognition | 23502 |
| | chain | | of a highly conserved influenza virus | |
| | | | epitope; Science 324 (5924), 246-251 | |
| | | | (2009), NCBI Accession #3GBN_H | |
| INFL8 | Fab lambda light | CR6261 | Ekiert, D. C. et al., Antibody recognition | 23503 |
| | chain | | of a highly conserved influenza virus | |
| | | | epitope; Science 324 (5924), 246-251 | |
| | | | (2009), NCBI Accession #3GBN_L | |
| INFL9 | Fab lambda light | Fab Cr6261 | Lingwood, D., et al., Structural and | 23504 |
| | chain | (Somatic Heavy | genetic basis for development of broadly | |
| | | Chain With | neutralizing influenza antibodies; Nature | |
| | | Germline- | 489 (7417), 566-570 (2012), NCBI | |
| | | Reverted Light | Accession #4EVN_N (217aa) | |
| | | Chain) | | |
| INFL10 | Fab light chain | Del2d1 | Krause, J. C. et al., M Bio 2 (1), E00345- | 23505 |
| | | | E00310 (2011), NCBI Accession | |
| | | | #3QHF_L | |
| INFL11 | Fab Light Chain | Fld194 Fab | Xiong, X. et al., Structures of complexes | 23506 |
| | | | formed by H5 influenza hemagglutinin | |
| | | | with a potent broadly neutralizing human | |
| | | | monoclonal antibody; Proc. Natl. Acad, | |
| | | | Sci. U.S.A. 112 (30), 9430-9435 (2015), | |
| | | | NCBI Accession #5A3I_D (219aa) | |
| INFL12 | Fab, heavy chain | F045-092 | Lee, P. S. et al., Receptor mimicry by | 23507 |
| | | | antibody F045-092 facilitates universal | |
| | | | binding to the H3 subtype of influenza | |
| | | | virus; Nat Commun 5, 3614 (2014), | |
| | | | NCBI Accession #4O5I_W | |
| INFL13 | Fab, Light Chain | F045-092 | Lee, P. S. et al., Receptor mimicry by | 23508 |
| | | | antibody F045-092 facilitates universal | |
| | | | binding to the H3 subtype of influenza | |
| | | | virus; Nat Commun 5, 3614 (2014), | |
| | | | NCBI Accession #4O5I_V | |
| INFL14 | Fab, light chain | H5.3 | Winarski, K. L., Thornburg, N. J. et al., | 23509 |
| | | | “Vaccine-elicited antibody that | |
| | | | neutralizes H5N1 influenza and variants | |
| | | | binds the receptor site and polymorphic | |
| | | | sites “PNAS 2015 112 (30) 9346-9351”, | |
| | | | NCBI Accession #4XNM_L | |
| INFL15 | Gamma heavy chain | 8i10 | U.S. Pat. No. 8,858,948 SEQ ID NO: 69 | 23510 |
| | variable | | | |
| INFL16 | Gamma heavy chain | 23K12 | U.S. Pat. No. 8,858,948 SEQ ID NO: 100 | 23511 |
| | variable | | | |
| INFL17 | Heavy chain | CR6261, | WO 2008028946 | 23512 |
| | | Diridavumab, CR- | | |
| | | 6261 | | |
| INFL18 | Heavy chain | Firivumab, CT-P22 | US20130004505 | 23513 |
| INFL19 | Heavy chain | CT-P22 | US20130004505 SEQ ID NO: 41; WO | 23514 |
| | | | 2011/111966 | |
| INFL20 | Heavy chain | Navivumab, | WO2013048153, US20140234336 SEQ | 23515 |
| | | CT149 | ID NO: 40 | |
| INFL21 | Heavy chain | AT10-004 | US20150010566, WO2013081463 SEQ | 23516 |
| | | | ID NO: 31 | |
| INFL22 | Heavy chain | AT10-003 | US20150010566, WO2013081463 SEQ | 23517 |
| | | | ID NO: 32 | |
| INFL23 | Heavy chain | AT10-002 | US20150010566, WO2013081463 SEQ | 23518 |
| | | | ID NO: 33 | |
| INFL24 | Heavy chain | AT10-001 | US20150010566, WO2013081463 SEQ | 23519 |
| | | | ID NO: 34 | |
| INFL25 | Heavy chain | AT10-005 | US20150010566, WO2013081463 SEQ | 23520 |
| | | | ID NO: 35 | |
| INFL26 | Heavy chain | CT104 | WO2011111966, US20130004505 SEQ | 23521 |
| | | | ID NO: 37 | |
| INFL27 | Heavy chain | CT120 | WO2011111966, US20130004505 SEQ | 23522 |
| | | | ID NO: 41 | |
| INFL28 | Heavy chain | CT123 | WO2011111966, US20130004505 SEQ | 23523 |
| | | | ID NO: 45 | |
| INFL29 | Heavy chain | 2A | US20140011982 SEQ ID NO: 2 | 23524 |
| INFL30 | Heavy chain | F005-126 | WO2014049520, US20140086927 SEQ | 23525 |
| | | | ID NO: 2 | |
| INFL31 | Heavy chain | BF1-1 | WO2008156763 SEQ ID NO: 7 | 23526 |
| INFL32 | Heavy chain | BF1-19 | WO2008156763 SEQ ID NO: 11 | 23527 |
| INFL33 | Heavy chain | BF1-10 | WO2008156763 SEQ ID NO: 9 | 23528 |
| INFL34 | Heavy chain | | WO2010127252, U.S. Pat. No. 8,894,997 SEQ | 23529 |
| | | | ID NO: 3 | |
| INFL35 | Heavy chain | A18 | WO13170139 SEQ ID NO: 94 | 23530 |
| INFL36 | Heavy chain | Ab A18 | U.S. Pat. No. 7,788,200 SEQ ID NO: 15 | 23531 |
| INFL37 | Heavy chain | Ab 014, Ab 028 | U.S. Pat. No. 7,788,200 SEQ ID NO: 16 | 23532 |
| INFL38 | Heavy chain | Ab 071 | U.S. Pat. No. 7,788,200 SEQ ID NO: 162 | 23533 |
| INFL39 | Heavy chain | Ab 072 | U.S. Pat. No. 7,788,200 SEQ ID NO: 163 | 23534 |
| INFL40 | Heavy chain | Ab 078, Ab 079, | U.S. Pat. No. 7,788,200 SEQ ID NO: 164 | 23535 |
| | | Ab 080, Ab 081 | | |
| INFL41 | Heavy chain | Ab 001, Ab 009, | U.S. Pat. No. 7,788,200 SEQ ID NO: 17 | 23536 |
| | | Ab 017, Ab 160, | | |
| | | Ab 186, Ab 187, | | |
| | | Ab 188, Ab 189, | | |
| | | Ab 190, Ab 191, | | |
| | | Ab 192, Ab 193, | | |
| | | Ab 202, Ab 211 | | |
| INFL42 | Heavy chain | Ab 002, Ab 010, | U.S. Pat. No. 7,788,200 SEQ ID NO: 18 | 23537 |
| | | Ab 026, Ab 203, | | |
| | | Ab 212 | | |
| INFL43 | Heavy chain | Ab 003, Ab 011, | U.S. Pat. No. 7,788,200 SEQ ID NO: 19 | 23538 |
| | | Ab 027, Ab 194, | | |
| | | Ab 195, Ab 196, | | |
| | | Ab 197, Ab 198, | | |
| | | Ab 199, Ab 200, | | |
| | | Ab 204, Ab 213 | | |
| INFL44 | Heavy chain | Ab 086 | U.S. Pat. No. 7,788,200 SEQ ID NO: 20 | 23539 |
| INFL45 | Heavy chain | Ab 154, Ab 155, | U.S. Pat. No. 7,788,200 SEQ ID NO: 21 | 23540 |
| | | Ab 157 | | |
| INFL46 | Heavy chain | Ab 157, Ab 159 | U.S. Pat. No. 7,788,200 SEQ ID NO: 22 | 23541 |
| INFL47 | Heavy chain | Ab 210, Ab 219 | U.S. Pat. No. 7,788,200 SEQ ID NO: 23 | 23542 |
| INFL48 | Heavy chain | Ab A001, Ab | U.S. Pat. No. 7,788,200 SEQ ID NO: 24 | 23543 |
| | | A002, Ab A003, | | |
| | | Ab A010, Ab | | |
| | | A011, Ab 031, Ab | | |
| | | 037 | | |
| INFL49 | Heavy chain | Ab 004, Ab 005, | U.S. Pat. No. 7,788,200 SEQ ID NO: 25 | 23544 |
| | | Ab 006, Ab 012, | | |
| | | Ab 013, Ab 032, | | |
| | | Ab 038, Ab 043, | | |
| | | Ab 044, Ab 045, | | |
| | | Ab 046, Ab 047, | | |
| | | Ab 048, Ab 049, | | |
| | | Ab 050, Ab 051, | | |
| | | Ab 052, Ab 067, | | |
| | | Ab 068, Ab 069, | | |
| | | Ab 070, Ab 073, | | |
| | | Ab 074, Ab 075, | | |
| | | Ab 076, Ab 077 | | |
| INFL50 | Heavy chain | Ab 007, Ab 008, | U.S. Pat. No. 7,788,200 SEQ ID NO: 26 | 23545 |
| | | Ab A009, Ab A14, | | |
| | | Ab 015, Ab 033, | | |
| | | Ab 039 | | |
| INFL51 | Heavy chain | Ab 016, Ab A017, | U.S. Pat. No. 7,788,200 SEQ ID NO: 27 | 23546 |
| | | Ab C18, Ab A019, | | |
| | | Ab 034, Ab 040 | | |
| INFL52 | Heavy chain | F005-126 | WO2014049520 SEQ ID 2 | 23547 |
| INFL53 | Heavy chain | 8f24 | WO2012045001 SEQ ID 1 | 23548 |
| INFL54 | Heavy chain | 3E22 | WO2012045001 SEQ ID 5 | 23549 |
| INFL55 | Heavy chain | 5117 | WO2012045001 SEQ ID 9 | 23550 |
| INFL56 | Heavy chain | | WO2012045001 SEQ ID 13 | 23551 |
| INFL57 | Heavy chain | | WO2012045001 SEQ ID 29 | 23552 |
| INFL58 | Heavy chain | | WO2012045001 SEQ ID 33 | 23553 |
| INFL59 | Heavy chain | | WO2012045001 SEQ ID 17 | 23554 |
| INFL60 | Heavy chain | 10A14 | WO2012045001 SEQ ID 21 | 23555 |
| INFL61 | Heavy chain | 8D4 | WO2012045001 SEQ ID 25 | 23556 |
| INFL62 | Heavy chain | 2B9 | U.S. Pat. No. 9,115,201 SEQ ID NO: 6 | 23557 |
| INFL63 | Heavy chain | mAB 7A7 | US20150239960, US20140170163, | 23558 |
| | | | U.S. Pat. No. 8,673,314, US20110027270, | |
| | | | WO2010138564 SEQ ID NO: 6 | |
| INFL64 | Heavy chain | mAB 12D1 | US20150239960, US20140170163, | 23559 |
| | | | U.S. Pat. No. 8,673,314, US20110027270, | |
| | | | WO2010138564 SEQ ID NO: 12 | |
| INFL65 | Heavy chain | mAB 66A6 | US20150239960, US20140170163, | 23560 |
| | | | U.S. Pat. No. 8,673,314, US20110027270, | |
| | | | WO2010138564 SEQ ID NO: 16 | |
| INFL66 | Heavy chain | M1 D12 | US20110033473, WO2009125395 SEQ | 23561 |
| | | | ID NO: 17 | |
| INFL67 | Heavy chain | mAB1.12 | WO2013030165 SEQ ID NO: 1 | 23562 |
| INFL68 | Heavy chain | mAB3.1 | WO2013030165 SEQ ID NO: 3 | 23563 |
| INFL69 | Heavy chain | 5A7 | WO2015120097 SEQ ID NO: 7 | 23564 |
| INFL70 | Heavy chain | TRL053 | WO2015120097 SEQ ID NO: 17 | 23565 |
| INFL71 | Heavy chain | TRL579 | WO2015120097 SEQ ID NO: 27 | 23566 |
| INFL72 | Heavy chain | TRL784 | WO2015120097 SEQ ID NO: 37 | 23567 |
| INFL73 | Heavy chain | TRL794 | WO2015120097 SEQ ID NO: 47 | 23568 |
| INFL74 | Heavy chain | TRL798 | WO2015120097 SEQ ID NO: 57 | 23569 |
| INFL75 | Heavy chain | TRL799 | WO2015120097 SEQ ID NO: 67 | 23570 |
| INFL76 | Heavy chain | TRL809 | WO2015120097 SEQ ID NO: 77 | 23571 |
| INFL77 | Heavy chain | TRL811 | WO2015120097 SEQ ID NO: 87 | 23572 |
| INFL78 | Heavy chain | TRL812 | WO2015120097 SEQ ID NO: 97 | 23573 |
| INFL79 | Heavy chain | TRL813 | WO2015120097 SEQ ID NO: 107 | 23574 |
| INFL80 | Heavy chain | TRL823 | WO2015120097 SEQ ID NO: 117 | 23575 |
| INFL81 | Heavy chain | TRL832 | WO2015120097 SEQ ID NO: 127 | 23576 |
| INFL82 | Heavy chain | TRL833 | WO2015120097 SEQ ID NO: 137 | 23577 |
| INFL83 | Heavy chain | TRL834 | WO2015120097 SEQ ID NO: 147 | 23578 |
| INFL84 | Heavy chain | TRL835 | WO2015120097 SEQ ID NO: 157 | 23579 |
| INFL85 | Heavy chain | TRL835 | WO2015120097 SEQ ID NO: 158 | 23580 |
| INFL86 | Heavy chain | TRL837 | WO2015120097 SEQ ID NO: 168 | 23581 |
| INFL87 | Heavy chain | TRL839 | WO2015120097 SEQ ID NO: 178 | 23582 |
| INFL88 | Heavy chain | TRL841 | WO2015120097 SEQ ID NO: 188 | 23583 |
| INFL89 | Heavy chain | TRL842 | WO2015120097 SEQ ID NO: 198 | 23584 |
| INFL90 | Heavy chain | TRL845 | WO2015120097 SEQ ID NO: 208 | 23585 |
| INFL91 | Heavy chain | TRL846 | WO2015120097 SEQ ID NO: 217 | 23586 |
| INFL92 | Heavy chain | TRL847 | WO2015120097 SEQ ID NO: 227 | 23587 |
| INFL93 | Heavy chain | TRL848 | WO2015120097 SEQ ID NO: 237 | 23588 |
| INFL94 | Heavy chain | TRL849 | WO2015120097 SEQ ID NO: 247 | 23589 |
| INFL95 | Heavy chain | TRL851 | WO2015120097 SEQ ID NO: 257 | 23590 |
| INFL96 | Heavy chain | TRL854 | WO2015120097 SEQ ID NO: 267 | 23591 |
| INFL97 | Heavy chain | TRL856 | WO2015120097 SEQ ID NO: 277 | 23592 |
| INFL98 | Heavy chain | TRL858 | WO2015120097 SEQ ID NO: 287 | 23593 |
| INFL99 | Heavy chain | humM2e-hBiTE-1 | WO2014140368 SEQ ID NO: 8 | 23594 |
| INFL100 | Heavy chain | humM2e-hBiTE-2 | WO2014140368 SEQ ID NO: 16 | 23595 |
| INFL101 | Heavy chain | humM2e-hBiTE-3 | WO2014140368 SEQ ID NO: 24 | 23596 |
| INFL102 | Heavy chain | humM2e-hBiTE-4 | WO2014140368 SEQ ID NO: 32 | 23597 |
| INFL103 | Heavy chain | VH of humM2e- | WO2014140368 SEQ ID NO: 40 | 23598 |
| | | hBiTE-5 | | |
| INFL104 | Heavy chain | humM2e-hBiTE-6 | WO2014140368 SEQ ID NO: 48 | 23599 |
| INFL105 | Heavy chain | humM2e-hBiTE-7 | WO2014140368 SEQ ID NO: 56 | 23600 |
| INFL106 | Heavy chain | humM2e-hBiTE-8 | WO2014140368 SEQ ID NO: 64 | 23601 |
| INFL107 | Heavy chain | humM2e-hBiTE-9 | WO2014140368 SEQ ID NO: 72 | 23602 |
| INFL108 | Heavy chain | murM2e-hBiTE | WO2014140368 SEQ ID NO: 80 | 23603 |
| INFL109 | Heavy chain | FLA5.10 | U.S. Pat. No. 8,124,092 SEQ ID NO: 1 | 23604 |
| INFL110 | Heavy chain | FLD21.140 | U.S. Pat. No. 8,124,092 SEQ ID NO: 5 | 23605 |
| INFL111 | Heavy chain | FLA3.14 | U.S. Pat. No. 8,124,092 SEQ ID NO: 9 | 23606 |
| INFL112 | Heavy chain | FLD20.19 | U.S. Pat. No. 8,124,092 SEQ ID NO: 13 | 23607 |
| INFL113 | Heavy chain | FLD84 | U.S. Pat. No. 8,124,092 SEQ ID NO: 42 | 23608 |
| INFL114 | Heavy chain | FLD93 | U.S. Pat. No. 8,124,092 SEQ ID NO: 52 | 23609 |
| INFL115 | Heavy chain | FLD122 | U.S. Pat. No. 8,124,092 SEQ ID NO: 62 | 23610 |
| INFL116 | Heavy chain | FLD127 | U.S. Pat. No. 8,124,092 SEQ ID NO: 72 | 23611 |
| INFL117 | Heavy chain | FLD129 | U.S. Pat. No. 8,124,092 SEQ ID NO: 82 | 23612 |
| INFL118 | Heavy chain | FLD132 | U.S. Pat. No. 8,124,092 SEQ ID NO: 92 | 23613 |
| INFL119 | Heavy chain | FLD194 | U.S. Pat. No. 8,124,092 SEQ ID NO: 102 | 23614 |
| INFL120 | Heavy chain | mAb2 | WO2015112994 SEQ ID NO: 80 | 23615 |
| INFL121 | Heavy chain | mAb3 | WO2015112994 SEQ ID NO: 84 | 23616 |
| INFL122 | Heavy chain | | Tsibane, T. et al., Influenza Human | 23617 |
| | | | Monoclonal Antibody 1F1 Interacts with | |
| | | | Three Major Antigenic Sites and | |
| | | | Residues Mediating Human Receptor | |
| | | | Specificity in H1N1 Viruses; PLoS | |
| | | | Pathol. 8 (12), E1003067 (2012), NCBI | |
| | | | Accession #4GXU_S | |
| INFL123 | Heavy chain | C05 | Ekiert, D. C., et al., Cross-neutralization | 23618 |
| | | | of influenza A viruses mediated by a | |
| | | | single antibody loop; Nature 489 (7417), | |
| | | | 526-532 (2012), NCBI Accession | |
| | | | #4FNL_H (247aa) | |
| INFL124 | Heavy chain | CR8020 | Ekiert, D. C., et al., A. highly conserved | 23619 |
| | | | neutralizing epitope on group 2 influenza | |
| | | | A viruses; Science 333 (6044), 843-850 | |
| | | | (2011); WO2010130636, NCBI | |
| | | | Accession #3SDY_H | |
| INFL125 | Heavy chain | CR8043 | Friesen, R. H. et al., A common solution | 23620 |
| | | | to group 2 influenza virus neutralization; | |
| | | | Proc. Natl. Acad. Sci. U.S.A. 111 (1), | |
| | | | 445-450 (2014), NCBI Accession | |
| | | | #4NM8_H | |
| INFL126 | Heavy chain | CR8059 | Dreyfus, C. et al., Highly conserved | 23621 |
| | | | protective epitopes on influenza B | |
| | | | viruses; Science 337 (6100), 1343-1348 | |
| | | | (2012), NCBI Accession #4FQK_H | |
| INFL127 | Heavy chain | CR8071 | Dreyfus, C. et al., Highly conserved | 23622 |
| | | | protective epitopes on influenza B | |
| | | | viruses; Science 337 (6100), 1343-1348 | 23623 |
| | | | (2012), NCBI Accession #4FQJ_H | |
| | | | (234aa) | |
| INFL128 | Heavy chain | CR9114 | WO2013079473; WO2014191435; | 23624 |
| | | | Dreyfus, C., Laursen, N. S. et al., Highly | |
| | | | conserved protective epitopes on | |
| | | | influenza B viruses; Science 337 (6100), | |
| | | | 1343-1348 (2012), NCBI Accession | |
| | | | #4FQY_H (230aa) | |
| INFL129 | Heavy chain | Ch67 | Schmidt, A. G., et al., Preconfiguration of | 23625 |
| | | | the antigen-binding site during affinity | |
| | | | maturation of a broadly neutralizing | |
| | | | influenza virus antibody; Proc. Natl. | |
| | | | Acad. Set. U.S.A. 110 (1), 264-269 | |
| | | | (2013), NCBI Accession #4HKX_A | |
| | | | (231aa) | |
| INFL130 | Heavy chain | Fab 26/9 | Schulze-Gahmen, U. et al., J. Biol. | 23626 |
| | | | Chem. 263 (32), 17100-17105 (1988); | |
| | | | Churchill, M. E., et al., J. Mol. Biol. 241 | |
| | | | (4), 534-556 (1994), NCBI Accession | |
| | | | #1FRG_H | |
| INFL131 | Heavy chain | Fab 3.1 | Wyrzucki, A. et al., Alternative | 23627 |
| | | | Recognition of the Conserved Stem | |
| | | | Epitope in Influenza A Virus | |
| | | | Hemagglutinin by a VH3-30-Encoded | |
| | | | Heterosubtypic Antibody; J. Virol. 88 | |
| | | | (12), 7083-7092 (2014), NCBI Accession | |
| | | | #4PY8_I | |
| INFL132 | Heavy chain | Fab 2g1 | Xu, R. et al., A recurring motif for | 23628 |
| | | | antibody recognition of the receptor- | |
| | | | binding site of influenza hemagglutinin; | |
| | | | Nat. Struct. Mol. Biol. 20 (3), 363-370 | |
| | | | (2013), NCBI Accession #4HG4_N | |
| | | | (223aa) | |
| INFL133 | Heavy chain | Fab 8m2 | Xu, R. et al., A recurring motif for | 23629 |
| | | | antibody recognition of the receptor- | |
| | | | binding site of influenza hemagglutinin; | |
| | | | Nat. Struct. Mol. Biol. 20 (3), 363-370 | |
| | | | (2013), NCBI Accession #4HFU_H | |
| | | | (226aa) | |
| INFL134 | Heavy chain | Fab 8f8 | Xu, R. et al., A recurring motif for | 23630 |
| | | | antibody recognition of the receptor- | |
| | | | binding site of influenza hemagglutinin; | |
| | | | Nat. Struct. Mol. Biol. 20 (3), 363-370 | |
| | | | (2013), NCBI Accession #4HF5_H | |
| | | | (233aa) | |
| INFL135 | Heavy chain | Fab 2d1 | Xu, R., et al., Structural basis of | 23631 |
| | | | preexisting immunity to the 2009 H1N1 | |
| | | | pandemic influenza virus; Science 328 | |
| | | | (5976), 357-360 (2010), NCBI Accession | |
| | | | #3LZF_H (230aa) | |
| INFL136 | Heavy chain | Fi6v3 | Corti, D. et al., A neutralizing antibody | 23632 |
| | | | selected from plasma cells that binds to | |
| | | | group 1 and group 2 influenza A | |
| | | | hemagglutinins; Science 333 (6044), | |
| | | | 850-856 (2011), NCBI Accession | |
| | | | #3ZTJ_G | |
| INFL137 | Heavy Chain | Heavy chain | Iba, Y., et al., Conserved Neutralizing | 23633 |
| | | 3WHE_N | Epitope at Globular Head of | |
| | | | Hemagglutinin in H3N2 Influenza | |
| | | | Viruses; J. Virol. (2014), NCBI | 23634 |
| | | | Accession #3WHE_M (226aa) | |
| INFL138 | Heavy chain | 7A13 | Krause et al. “Human Monoclonal | 23635 |
| | | | Antibodies to Pandemic 1957 H2N2 and | |
| | | | Pandemic 1968 H3N2 Influenza Viruses” | |
| | | | J. Virol. 86 (11), 6334-6340 (2012), | |
| | | | NCBI Accession #AFH78447 | |
| INFL139 | Heavy chain | 2D1 | WO2010127252, U.S. Pat. No. 8,894,997 SEQ ID | 23636 |
| | | | NO: 7 | |
| INFL140 | Heavy chain | 1F1 | WO2010127252, U.S. Pat. No. 8,894,997 SEQ ID | 23637 |
| | | | NO: 1 | |
| INFL141 | Heavy chain | | WO2010127252, U.S. Pat. No. 8,894,997 SEQ ID | 23638 |
| | | | NO: 4 | |
| INFL142 | Heavy chain | 1I20 | WO2010127252, U.S. Pat. No. 8,894,997 SEQ ID | 23639 |
| | | | NO: 5 | |
| INFL143 | Heavy chain | 4D20 | WO2010127252, U.S. Pat. No. 8,894,997 SEQ ID | 23640 |
| | | | NO: 9 | |
| INFL144 | Heavy chain | | WO2010127252, U.S. Pat. No. 8,894,997 SEQ ID | 23641 |
| | | | NO: 11 | |
| INFL145 | Heavy chain | | US20140205614, US20100316654 SEQ | 23642 |
| | | | ID NO: 21 | |
| INFL146 | Heavy chain | | US20140205614, US20100316654 SEQ | 23643 |
| | | | ID NO: 22 | |
| INFL147 | Heavy chain | | US20140205614, US20100316654 SEQ | 23644 |
| | | | ID NO: 23 | |
| INFL148 | Heavy chain | | US20140205614, US20100316654 SEQ | 23645 |
| | | | ID NO: 24 | |
| INFL149 | Heavy chain | | US20140205614, US20100316654 SEQ | 23646 |
| | | | ID NO: 25 | |
| INFL150 | Heavy chain | | US20140205614, US20100316654 SEQ | 23647 |
| | | | ID NO: 26 | |
| INFL151 | Heavy chain | | US20140205614, US20100316654 SEQ | 23648 |
| | | | ID NO: 27 | |
| INFL152 | Heavy chain | | US20140205614, US20100316654 SEQ | 23649 |
| | | | ID NO: 28 | |
| INFL153 | Heavy chain | | US20140205614, US20100316654 SEQ | 23650 |
| | | | ID NO: 29 | |
| INFL154 | Heavy chain | | US20140205614, US20100316654 SEQ | 23651 |
| | | | ID NO: 30 | |
| INFL155 | Heavy chain | | US20140205614, US20100316654 SEQ | 23652 |
| | | | ID NO: 31 | |
| INFL156 | Heavy chain | | US20140205614, US20100316654 SEQ | 23653 |
| | | | ID NO: 32 | |
| INFL157 | Heavy chain | | US20140205614, US20100316654 SEQ | 23654 |
| | | | ID NO: 33 | |
| INFL158 | Heavy chain | | US20140205614, US20100316654 SEQ | 23655 |
| | | | ID NO: 34 | |
| INFL159 | Heavy chain | | US20140205614, US20100316654 SEQ | 23656 |
| | | | ID NO: 35 | |
| INFL160 | Heavy chain | | US20140205614, US20100316654 SEQ | 23657 |
| | | | ID NO: 36 | |
| INFL161 | Heavy chain | | US20140205614, US20100316654 SEQ | 23658 |
| | | | ID NO: 37 | |
| INFL162 | Heavy chain | | US20140205614, US20100316654 SEQ | 23659 |
| | | | ID NO: 38 | |
| INFL163 | Heavy chain | | US20140205614, US20100316654 SEQ | 23660 |
| | | | ID NO: 39 | |
| INFL164 | Heavy chain | | US20140205614, US20100316654 SEQ | 23661 |
| | | | ID NO: 40 | |
| INFL165 | Heavy chain | | US20140205614, US20100316654 SEQ | 23662 |
| | | | ID NO: 41 | |
| INFL166 | Heavy chain | | US20140205614, US20100316654 SEQ | 23663 |
| | | | ID NO: 42 | |
| INFL167 | Heavy chain | | US20140205614, US20100316654 SEQ | 23664 |
| | | | ID NO: 43 | |
| INFL168 | Heavy chain | | US20140205614, US20100316654 SEQ | 23665 |
| | | | ID NO: 44 | |
| INFL169 | Heavy chain | | US20140205614, US20100316654 SEQ | 23666 |
| | | | ID NO: 45 | |
| INFL170 | Heavy chain | mAb1 | WO2015112994 SEQ ID NO: 76 | 23667 |
| INFL171 | Heavy chain | CR8033 | Dreyfus, C., Laursen, N. S. et al., Highly | 23668 |
| | | | conserved protective epitopes on | |
| | | | influenza B viruses; Science 337 (6100), | |
| | | | 1343-1348 (2012), NCBI Accession # | |
| | | | 4FQL_H | |
| INFL172 | Heavy chain (Partial) | monoclonal | Burioni, R. et al., Monoclonal antibodies | 23669 |
| | | antibody PN- | isolated from human B cells neutralize a | |
| | | SIA28 | broad range of H1 subtype influenza A | |
| | | | viruses including swine-origin Influenza | |
| | | | virus(S-OIV); Virology (2010), NCBI | |
| | | | Accession #ACX30936.1 (122aa) | |
| INFL173 | Heavy chain (Partial) | monoclonal | Burioni, R, et al., Monoclonal antibodies | 23670 |
| | | antibody PN- | isolated from human B cells neutralize a | |
| | | SIA49 | broad range of H1 subtype influenza A | |
| | | | viruses including swine-origin Influenza | |
| | | | virus(S-OIV); Virology (2010), NCBI | |
| | | | Accession #ACX30937.1 (127aa) | |
| INFL174 | Heavy chain cdr1 | Ab1A2 | WO2015028478 SEQ ID 6 | 23671 |
| INFL175 | Heavy chain cdr2 | Ab1A2 | WO2015028478 SEQ ID 7 | 23672 |
| INFL176 | Heavy chain cdr3 | Ab1A2 | WO2015028478 SEQ ID 8 | 23673 |
| INFL177 | Heavy chain constant | | U.S. Pat. No. 8,992,929 SEQ ID NO. 22 | 23674 |
| | region, Human igg1 | | | |
| INFL178 | Heavy chain Fab | CT147 | WO2013048153, US20140234336 SEQ | 23675 |
| | | | ID NO: 38 | |
| INFL179 | Heavy chain Fab | CT164 | WO2013048153, US20140234336 SEQ | 23676 |
| | | | ID NO: 42 | |
| INFL180 | Heavy chain Fab | CT166 | WO2013048153, US20140234336 SEQ | 23677 |
| | | | ID NO: 44 | |
| INFL181 | Heavy chain G2 | h2B9 | U.S. Pat. No. 9,115,201 SEQ ID NO: 7 | 23678 |
| INFL182 | Heavy chain G5 | h2B10 | U.S. Pat. No. 9,115,201 SEQ ID NO: 8 | 23679 |
| INFL183 | Heavy chain variable | HC-VD from | US2013030234 SEQ ID NO: 1 | 23680 |
| | (exemplary) | US2013030234 | | |
| INFL184 | Heavy chain variable | HC-VD from | US2013030234 SEQ ID NO: 2 | 23681 |
| | (exemplary) | US2013030234 | | |
| INFL185 | Heavy chain variable | HC-VD from | US2013030234 SEQ ID NO: 3 | 23682 |
| | (exemplary) | US2013030234 | | |
| INFL186 | Heavy chain variable | HC-VD from | US2013030234 SEQ ID NO: 4 | 23683 |
| | (exemplary) | US2013030234 | | |
| INFL187 | Heavy chain variable | HC-VD from | US2013030234 SEQ ID NO: 5 | 23684 |
| | (exemplary) | US2013030234 | | |
| INFL188 | Heavy chain variable | HC-VD from | US2013030234 SEQ ID NO: 6 | 23685 |
| | (exemplary) | US2013030234 | | |
| INFL189 | Heavy chain variable | HC-VD from | US2013030234 SEQ ID NO: 7 | 23686 |
| | (exemplary) | US2013030234 | | |
| INFL190 | Heavy chain variable | HC-VD from | US2013030234 SEQ ID NO: 8 | 23687 |
| | (exemplary) | US2013030234 | | |
| INFL191 | Heavy chain variable | HC-VD from | US2013030234 SEQ ID NO: 9 | 23688 |
| | (exemplary) | US2013030234 | | |
| INFL192 | Heavy chain variable | HC-VD from | US2013030234 SEQ ID NO: 10 | 23689 |
| | (exemplary) | US2013030234 | | |
| INFL193 | Heavy chain variable | HC-VD from | US2013030234 SEQ ID NO: 11 | 23690 |
| | (exemplary) | US2013030234 | | |
| INFL194 | Heavy chain variable | HC-VD from | US2013030234 SEQ ID NO: 12 | 23691 |
| | (exemplary) | US2013030234 | | |
| INFL195 | Heavy chain variable | HC-VD from | US2013030234 SEQ ID NO: 13 | 23692 |
| | (exemplary) | US2013030234 | | |
| INFL196 | Heavy chain variable | HC-VD from | US2013030234 SEQ ID NO: 14 | 23693 |
| | (exemplary) | US2013030234 | | |
| INFL197 | Heavy chain variable | HC-VD from | US2013030234 SEQ ID NO: 15 | 23694 |
| | (exemplary) | US2013030234 | | |
| INFL198 | Heavy chain variable | HC-VD from | US2013030234 SEQ ID NO: 16 | 23695 |
| | (exemplary) | US2013030234 | | |
| INFL199 | Heavy chain variable | CR6141 | US20150104459 SEQ ID NO: 199 | 23696 |
| | region | | | |
| INFL200 | Heavy chain variable | 39.18 B11 | US20140161822 SEQ ID NO: 154 | 23697 |
| | region | | | |
| INFL201 | Heavy chain variable | 39.18 E12 | US20140161822 SEQ ID NO: 158 | 23698 |
| | region | | | |
| INFL202 | Heavy chain variable | GG3 | WO2014159960 SEQ ID NO: 17 | 23699 |
| | region | | | |
| INFL203 | Heavy chain variable | N547 | U.S. Pat. No. 8,003,106 SEQ ID NO: 28 | 23700 |
| | region | | | |
| INFL204 | Heavy chain variable | L66 | U.S. Pat. No. 8,003,106 SEQ ID NO: 30 | 23701 |
| | region | | | |
| INFL205 | Heavy chain variable | C40 | U.S. Pat. No. 8,003,106 SEQ ID NO: 26 | 23702 |
| | region | | | |
| INFL206 | Heavy chain variable | 14C2 | U.S. Pat. No. 8,080,244 SEQ ID NO: 6 | 23703 |
| | region | | | |
| INFL207 | Heavy chain variable | h14C2 | U.S. Pat. No. 8,080,244 SEQ ID NO: 2 | 23704 |
| | region | | | |
| INFL208 | Heavy chain variable | 8G9 | U.S. Pat. No. 8,603,467 SEQ ID NO: 2 | 23705 |
| | region | | | |
| INFL209 | Heavy chain variable | 13D4 | U.S. Pat. No. 8,603,467 SEQ ID NO: 6 | 23706 |
| | region | | | |
| INFL210 | Heavy chain variable | 20A11 | U.S. Pat. No. 8,603,467 SEQ ID NO: 10 | 23707 |
| | region | | | |
| INFL211 | Heavy chain variable | VN04-2-HuG1 | US20100150941 SEQ ID NO: 5 | 23708 |
| | region | | | |
| INFL212 | Heavy chain variable | VN04-3-HuG1 | US20100150941 SEQ ID NO: 7 | 23709 |
| | region | | | |
| INFL213 | Heavy chain variable | FI6 variant 1 | U.S. Pat. No. 8,871,207 SEQ ID NO: 13 | 23710 |
| | region | | | |
| INFL214 | Heavy chain variable | FI6 variant 2 | U.S. Pat. No. 8,871,207 SEQ ID NO: 33 | 23711 |
| | region | | | |
| INFL215 | Heavy chain variable | FI6 variant 3 | U.S. Pat. No. 8,871,207 SEQ ID NO: 55 | 23712 |
| | region | | | |
| INFL216 | Heavy chain variable | FI6 variant 4, FI6 | U.S. Pat. No. 8,871,207 SEQ ID NO: 59 | 23713 |
| | region | variant 5 | | |
| INFL217 | Heavy chain variable | FI28 variant 1 | U.S. Pat. No. 8,871,207 SEQ ID NO: 29 | 23714 |
| | region | | | |
| INFL218 | Heavy chain variable | FI28 variant 2 | U.S. Pat. No. 8,871,207 SEQ ID NO: 35 | 23715 |
| | region | | | |
| INFL219 | Heavy chain variable | 21B15 | U.S. Pat. No. 8,858,948 SEQ ID NO: 44 | 23716 |
| | region | | | |
| INFL220 | Heavy chain variable | 3241_G23 | U.S. Pat. No. 8,858,948 SEQ ID NO: 116 | 23717 |
| | region | | | |
| INFL221 | Heavy chain variable | 3244_I10 | U.S. Pat. No. 8,858,948 SEQ ID NO: 120 | 23718 |
| | region | | | |
| INFL222 | Heavy chain variable | 3243_J07 | U.S. Pat. No. 8,858,948 SEQ ID NO: 124 | 23719 |
| | region | | | |
| INFL223 | Heavy chain variable | 3259_J21 | U.S. Pat. No. 8,858,948 SEQ ID NO: 128 | 23720 |
| | region | | | |
| INFL224 | Heavy chain variable | 3245_O19 | U.S. Pat. No. 8,858,948 SEQ ID NO: 132 | 23721 |
| | region | | | |
| INFL225 | Heavy chain variable | 3244_H04 | U.S. Pat. No. 8,858,948 SEQ ID NO: 136 | 23722 |
| | region | | | |
| INFL226 | Heavy chain variable | 3136_G05 | U.S. Pat. No. 8,858,948 SEQ ID NO: 140 | 23723 |
| | region | | | |
| INFL227 | Heavy chain variable | 3252_C13 | U.S. Pat. No. 8,858,948 SEQ ID NO: 144 | 23724 |
| | region | | | |
| INFL228 | Heavy chain variable | 3255_J06 | U.S. Pat. No. 8,858,948 SEQ ID NO: 148 | 23725 |
| | region | | | |
| INFL229 | Heavy chain variable | 3420_I23 | U.S. Pat. No. 8,858,948 SEQ ID NO: 152 | 23726 |
| | region | | | |
| INFL230 | Heavy chain variable | 3139_P23 | U.S. Pat. No. 8,858,948 SEQ ID NO: 156 | 23727 |
| | region | | | |
| INFL231 | Heavy chain variable | 3139_P23 | U.S. Pat. No. 8,858,948 SEQ ID NO: 158 | 23728 |
| | region | | | |
| INFL232 | Heavy chain variable | 3248_P18 | U.S. Pat. No. 8,858,948 SEQ ID NO: 162 | 23729 |
| | region | | | |
| INFL233 | Heavy chain variable | 3253_P10 | U.S. Pat. No. 8,858,948 SEQ ID NO: 166 | 23730 |
| | region | | | |
| INFL234 | Heavy chain variable | 3260_D19 | U.S. Pat. No. 8,858,948 SEQ ID NO: 170 | 23731 |
| | region | | | |
| INFL235 | Heavy chain variable | 3362_B11 | U.S. Pat. No. 8,858,948 SEQ ID NO: 172 | 23732 |
| | region | | | |
| INFL236 | Heavy chain variable | 3242_P05 | U.S. Pat. No. 8,858,948 SEQ ID NO: 176 | 23733 |
| | region | | | |
| INFL237 | Heavy chain variable | 2K11 | Krause, J. C. et al. “Epitope-specific | 23734 |
| | region | | human influenza antibody repertoires | |
| | | | diversify by B cell intraclonal sequence | |
| | | | divergence and interclonal convergence” | |
| | | | J. Immunol. 187 (7), 3704-3711 (2011), | |
| | | | NCBI Accession #AEO16793 | |
| INFL238 | Heavy chain variable | 2O10 | Krause, J. C. et al. “Epitope-specific | 23735 |
| | region | | human influenza antibody repertoires | |
| | | | diversify by B cell intraclonal sequence | |
| | | | divergence and interclonal convergence” | |
| | | | J. Immunol. 187 (7), 3704-3711 (2011), | |
| | | | NCBI Accession #AEO16795 | |
| INFL239 | Heavy chain variable | 4K8 | Krause, J. C. et al. “Epitope-specific | 23736 |
| | region | | human influenza antibody repertoires | |
| | | | diversify by B cell intraclonal sequence | |
| | | | divergence and interclonal convergence” | |
| | | | J. Immunol. 187 (7), 3704-3711 (2011), | |
| | | | NCBI Accession #AEO16799 | |
| INFL240 | Heavy chain variable | 6D9 | Krause, J. C. et al. “Epitope-specific | 23737 |
| | region | | human influenza antibody repertoires | |
| | | | diversify by B cell intraclonal sequence | |
| | | | divergence and interclonal convergence” | |
| | | | J. Immunol. 187 (7), 3704-3711 (2011), | |
| | | | NCBI Accession #AEO16801 | |
| INFL241 | Heavy chain variable | 4D20 | Yu, X. et al “Neutralizing antibodies | 23738 |
| | region | | derived from the B cells of 1918 | |
| | | | influenza pandemic survivors”, Nature | |
| | | | 455 (7212), 532-536, NCBI Accession | |
| | | | #ACI04579 | |
| INFL242 | Heavy chain variable | 2B12 | Yu, X. et al “Neutralizing antibodies | 23739 |
| | region | | derived from the B cells of 1918 | |
| | | | influenza pandemic survivors”, Nature | |
| | | | 455 (7212), 532-536, NCBI Accession | |
| | | | #ABY48866 | |
| INFL243 | Heavy chain variable | 8D4 | NCBI Accession #AFI57036 | 23740 |
| | region | | | |
| INFL244 | Heavy chain variable | 5B6 | NCBI Accession #AFI57040 | 23741 |
| | region | | | |
| INFL245 | Heavy chain variable | A66 | WO2009079259, US20110038935, | 23742 |
| | region | | US20140011982 SEQ ID NO: 32 | |
| INFL246 | Heavy chain variable | D7 | WO2009079259, US20110038935, | 23743 |
| | region | | US20140011982 SEQ ID NO: 6 | |
| INFL247 | Heavy chain variable | D8, D80 | WO2009079259, US20110038935, | 23744 |
| | region | | US20140011982 SEQ ID NO: 12 | |
| INFL248 | Heavy chain variable | E88 | WO2009079259, US20110038935, | 23745 |
| | region | | US20140011982 SEQ ID NO: 36 | |
| INFL249 | Heavy chain variable | E90, F10 | WO2009079259, US20110038935, | 23746 |
| | region | | US20140011982 SEQ ID NO: 18 | |
| INFL250 | Heavy chain variable | F10 | WO2009079259, US20110038935, | 23747 |
| | region | | US20140011982 SEQ ID NO: 112 | |
| INFL251 | Heavy chain variable | G17 | WO2009079259, US20110038935, | 23748 |
| | region | | US20140011982 SEQ ID NO: 24 | |
| INFL252 | Heavy chain variable | H40 | WO2009079259, US20110038935, | 23749 |
| | region | | US20140011982 SEQ ID NO: 28 | |
| INFL253 | Heavy chain variable | CH65 | WO2013020074, US20140302043 SEQ | 23750 |
| | region | | ID NO: 14 | |
| INFL254 | Heavy chain variable | CH66 | WO2013020074, US20140302043 SEQ | 23751 |
| | region | | ID NO: 15 | |
| INFL255 | Heavy chain variable | CH67 | WO2013020074, US20140302043 SEQ | 23752 |
| | region | | ID NO: 16 | |
| INFL256 | Heavy chain variable | CL86OUCA | WO2013020074, US20140302043 SEQ | 23753 |
| | region | | ID NO: 13 | |
| INFL257 | Heavy chain variable | Antibody 1 | WO2015051010 SEQ ID NO: 2 | 23754 |
| | region | | | |
| INFL258 | Heavy chain variable | Antibody 2 | WO2015051010 SEQ ID NO: 12 | 23755 |
| | region | | | |
| INFL259 | Heavy chain variable | Antibody 3 | WO2015051010 SEQ ID NO: 22 | 23756 |
| | region | | | |
| INFL260 | Heavy chain variable | Antibody 4 | WO2015051010 SEQ ID NO: 32 | 23757 |
| | region | | | |
| INFL261 | Heavy chain variable | Antibody 5 | WO2015051010 SEQ ID NO: 42 | 23758 |
| | region | | | |
| INFL262 | Heavy chain variable | Antibody 6 | WO2015051010 SEQ ID NO: 52 | 23759 |
| | region | | | |
| INFL263 | Heavy chain variable | Antibody 7 | WO2015051010 SEQ ID NO: 62 | 23760 |
| | region | | | |
| INFL264 | Heavy chain variable | Antibody 8 | WO2015051010 SEQ ID NO: 72 | 23761 |
| | region | | | |
| INFL265 | Heavy chain variable | Antibody 9 | WO2015051010 SEQ ID NO: 82 | 23762 |
| | region | | | |
| INFL266 | Heavy chain variable | Antibody 10 | WO2015051010 SEQ ID NO: 92 | 23763 |
| | region | | | |
| INFL267 | Heavy chain variable | Antibody 11 | WO2015051010 SEQ ID NO: 102 | 23764 |
| | region | | | |
| INFL268 | Heavy chain variable | Antibody 12 | WO2015051010 SEQ ID NO: 112 | 23765 |
| | region | | | |
| INFL269 | Heavy chain variable | Antibody 13 | WO2015051010 SEQ ID NO: 122 | 23766 |
| | region | | | |
| INFL270 | Heavy chain variable | Antibody 14 | WO2015051010 SEQ ID NO: 132 | 23767 |
| | region | | | |
| INFL271 | Heavy chain variable | Antibody 15 | WO201505I010 SEQ ID NO: 142 | 23768 |
| | region | | | |
| INFL272 | Heavy chain variable | Antibody 3-GL | WO2015051010 SEQ ID NO: 152 | 23769 |
| | region | | | |
| INFL273 | Heavy chain variable | EM4C04 | US20120282273 SEQ ID NO: 2 | 23770 |
| | region | | | |
| INFL274 | Heavy chain variable | 005-2G02 | WO2013059524, US20140348851 SEQ | 23771 |
| | region | | ID NO: 1 | |
| INFL275 | Heavy chain variable | 005-2G02 | WO2013059524, US20140348851 SEQ | 23772 |
| | region | | ID NO: 9 | |
| INFL276 | Heavy chain variable | 09-2A06 | WO2013059524, US20140348851 SEQ | 23773 |
| | region | | ID NO: 21 | |
| INFL277 | Heavy chain variable | 09-2A06 | WO2013059524, US20140348851 SEQ | 23774 |
| | region | | ID NO: 29 | |
| INFL278 | Heavy chain variable | 09-3A01 | WO2013059524, US20140348851 SEQ | 23775 |
| | region | | ID NO: 41 | |
| INFL279 | Heavy chain variable | 09-3A01 | WO2013059524, US20140348851 SEQ | 23776 |
| | region | | ID NO: 49 | |
| INFL280 | Heavy chain variable | 70-IF02 | WO2012096994, US20140046039 SEQ | 23777 |
| | region | | ID NO: 18 | |
| INFL281 | Heavy chain variable | | US20120058124 SEQ ID NO: 10 | 23778 |
| | region | | | |
| INFL282 | Heavy chain variable | | US20120058124 SEQ ID NO: 11 | 23779 |
| | region | | | |
| INFL283 | Heavy chain variable | | US20120058124 SEQ ID NO: 12 | 23780 |
| | region | | | |
| INFL284 | Heavy chain variable | | US20120058124 SEQ ID NO: 13 | 23781 |
| | region | | | |
| INFL285 | Heavy chain variable | | US20120058124 SEQ ID NO: 14 | 23782 |
| | region | | | |
| INFL286 | Heavy chain variable | 81.39 | US20140161822, US20140248286, | 23783 |
| | region | | WO2014078268 SEQ ID NO: 111 | |
| INFL287 | Heavy chain variable | 81.39 | US20140161822, US20140248286, | 23784 |
| | region | | WO2014078268 SEQ ID NO: 115 | |
| INFL288 | Heavy chain variable | 39.29 | US20140161822, US20140248286, | 23785 |
| | region | | WO2014078268 SEQ ID NO: 134 | |
| INFL289 | Heavy chain variable | 39.29 | US20140161822, US20140248286, | 23786 |
| | region | | WO2014078268 SEQ ID NO: 138 | |
| INFL290 | Heavy chain variable | 39.29 | US20140161822, US20140248286, | 23787 |
| | region | | WO2014078268 SEQ ID NO: 142 | |
| INFL291 | Heavy chain variable | 39.29 | US20140161822, US20140248286, | 23788 |
| | region | | WO2014078268 SEQ ID NO: 148 | |
| INFL292 | Heavy chain variable | 36.89 | US20140161822, US20140248286, | 23789 |
| | region | | WO2014078268 SEQ ID NO: 160 | |
| INFL293 | Heavy chain variable | 9.01F3 | US20140161822, US20140248286, | 23790 |
| | region | | WO2014078268 SEQ ID NO: 164 | |
| INFL294 | Heavy chain variable | 23.06C2 | US20140161822, US20140248286, | 23791 |
| | region | | WO2014078268 SEQ ID NO: 168 | |
| INFL295 | Heavy chain variable | 39.29 | US20140161822, US20140248286, | 23792 |
| | region | | WO2014078268 SEQ ID NO: 234 | |
| INFL296 | Heavy chain variable | F16 Variant 5 | WO2013011347, US20140271655, | 23793 |
| | region | | US8871207 SEQ ID NO: 59 | |
| INFL297 | Heavy chain variable | F16 Variant 3 | WO2013011347, US20140271655, | 23794 |
| | region | | US8871207 SEQ ID NO: 55 | |
| INFL298 | Heavy chain variable | F16 Variant 2 | WO2010010466 SEQ ID NO: 33 | 23795 |
| | region | | | |
| INFL299 | Heavy chain variable | FC41 | WO2010010467 SEQ ID NO 60 | 23796 |
| | region | | | |
| INFL300 | Heavy chain variable | FE43 | WO2010010467 SEQ ID NO 74 | 23797 |
| | region | | | |
| INFL301 | Heavy chain variable | FB75, FB110, | WO2010010467 SEQ ID NO 121 | 23798 |
| | region | FB177 | | |
| INFL302 | Heavy chain variable | FE17 | WO2010010467 SEQ ID NO 105 | 23799 |
| | region | | | |
| INFL303 | Heavy chain variable | FB79 | WO2010010467 SEQ ID NO 131 | 23800 |
| | region | | | |
| INFL304 | Heavy chain variable | FC1C | WO2010010467 SEQ ID NO 139 | 23801 |
| | region | | | |
| INFL305 | Heavy chain variable | FC6 | WO2010010467 SEQ ID NO 45 | 23802 |
| | region | | | |
| INFL306 | Heavy chain variable | FE53 | WO2010010467 SEQ ID NO 89 | 23803 |
| | region | | | |
| INFL307 | Heavy chain variable | 7A7 | WO2010138564 SEQ ID NO: 6 | 23804 |
| | region | | | |
| INFL308 | Heavy chain variable | 12DI | WO2010138564 SEQ ID NO: 12 | 23805 |
| | region | | | |
| INFL309 | Heavy chain variable | 66A6 | WO2010138564 SEQ ID NO: 16 | 23806 |
| | region | | | |
| INFL310 | Heavy chain variable | B-1 | U.S. Pat. No. 8,975,378, US20110319600, | 23807 |
| | region | | WO2010073647 SEQ ID NO: 27 | |
| INFL311 | Heavy chain variable | D1 | U.S. Pat. No. 8,975,378, US20110319600, | 23808 |
| | region | | WO2010073647 SEQ ID NO: 29 | |
| INFL312 | Heavy chain variable | E-2 | U.S. Pat. No. 8,975,378, US20110319600, | 23809 |
| | region | | WO2010073647 SEQ ID NO: 31 | |
| INFL313 | Heavy chain variable | B-3 | U.S. Pat. No. 8,975,378, US20110319600, | 23810 |
| | region | | WO2010073647 SEQ ID NO: 33 | |
| INFL314 | Heavy chain variable | 5A7 | WO2013114885, US20140377262 SEQ | 23811 |
| | region | | ID NO: 33 | |
| INFL315 | Heavy chain variable | 3A2 | WO2013114885, US20140377262 SEQ | 23812 |
| | region | | ID NO: 37 | |
| INFL316 | Heavy chain variable | 10C4 | WO2013114885, US20140377262 SEQ | 23813 |
| | region | | ID NO: 41 | |
| INFL317 | Heavy chain variable | Fab49 | WO2009144667, US20110076265 SEQ | 23814 |
| | region | | ID NO: 1 | |
| INFL318 | Heavy chain variable | Fab28 IgG PN- | WO2009115972, WO2011117848, | 23815 |
| | region | SIA28 | US20110014187 SEQ ID NO: 1 | |
| INFL319 | Heavy chain variable | TCN-522 | US20120207760, U.S. Pat. No. 8,916,160 SEQ ID | 23816 |
| | region | | NO: 771; U.S. Pat. No. 8,900,590 SEQ ID NO: 32 | |
| INFL320 | Heavy chain variable | CR8019 | WO2010130636 SEQ ID NO: 26 | 23817 |
| | region | | | |
| INFL321 | Heavy chain variable | CR8020 | WO2010130636 SEQ ID NO: 30 | 23818 |
| | region | | | |
| INFL322 | Heavy chain variable | CR8021 | WO2010130636 SEQ ID NO: 34 | 23819 |
| | region | | | |
| INFL323 | Heavy chain variable | CR8038 | WO2010130636 SEQ ID NO: 38 | 23820 |
| | region | | | |
| INFL324 | Heavy chain variable | CR8039 | WO2010130636 SEQ ID NO: 42 | 23821 |
| | region | | | |
| INFL325 | Heavy chain variable | CR8040 | WO2010130636 SEQ ID NO: 46 | 23822 |
| | region | | | |
| INFL326 | Heavy chain variable | CR8041 | WO2010130636 SEQ ID NO: 50 | 23823 |
| | region | | | |
| INFL327 | Heavy chain variable | CR8043 | WO2010130636 SEQ ID NO: 54 | 23824 |
| | region | | | |
| INFL328 | Heavy chain variable | CR8049 | WO2010130636 SEQ ID NO: 58 | 23825 |
| | region | | | |
| INFL329 | Heavy chain variable | CR8050 | WO2010130636 SEQ ID NO: 61 | 23826 |
| | region | | | |
| INFL330 | Heavy chain variable | CR8052 | WO2010130636 SEQ ID NO: 65 | 23827 |
| | region | | | |
| INFL331 | Heavy chain variable | CR8055 | WO2010130636 SEQ ID NO: 69 | 23828 |
| | region | | | |
| INFL332 | Heavy chain variable | CR8057 | WO2010130636 SEQ ID NO: 73 | 23829 |
| | region | | | |
| INFL333 | Heavy chain variable | CR8069 | WO2010130636 SEQ ID NO: 77 | 23830 |
| | region | | | |
| INFL334 | Heavy chain variable | CR6255 | US20090311265, U.S. Pat. No. 8,691,223, | 23831 |
| | region | | U.S. Pat. No. 9,109,017, WO2008028946A SEQ ID | |
| | | | NO: 59 | |
| INFL335 | Heavy chain variable | CR6257 | US20090311265, U.S. Pat. No. 8,691,223, | 23832 |
| | region | | U.S. Pat. No. 9,109,017, WO2008028946 SEQ ID | |
| | | | NO: 61 | |
| INFL336 | Heavy chain variable | CR6260 | US20090311265, U.S. Pat. No. 8,691,223, | 23833 |
| | region | | U.S. Pat. No. 9,109,017, WO2008028946 SEQ ID | |
| | | | NO: 63 | |
| INFL337 | Heavy chain variable | CR6261 | US20090311265, U.S. Pat. No. 8,691,223, | 23834 |
| | region | | U.S. Pat. No. 9,109,017, WO2008028946 SEQ ID | |
| | | | NO: 65 | |
| INFL338 | Heavy chain variable | CR6262 | US20090311265, U.S. Pat. No. 8,691,223, | 23835 |
| | region | | U.S. Pat. No. 9,109,017, WO2008028946 SEQ ID | |
| | | | NO: 67 | |
| INFL339 | Heavy chain variable | CR6268 | US20090311265, U.S. Pat. No. 8,691,223, | 23836 |
| | region | | U.S. Pat. No. 9,109,017, WO2008028946 SEQ ID | |
| | | | NO: 69 | |
| INFL340 | Heavy chain variable | CR6307 | US20090311265, U.S. Pat. No. 8,691,223, | 23837 |
| | region | | U.S. Pat. No. 9,109,017, WO2008028946 SEQ ID | |
| | | | NO: 71 | |
| INFL341 | Heavy chain variable | CR6310 | US20090311265, U.S. Pat. No. 8,691,223, | 23838 |
| | region | | U.S. Pat. No. 9,109,017, WO2008028946 SEQ ID | |
| | | | NO: 73 | |
| INFL342 | Heavy chain variable | CR6314 | US20090311265, U.S. Pat. No. 8,691,223, | 23839 |
| | region | | U.S. Pat. No. 9,109,017, WO2008028946 SEQ ID | |
| | | | NO: 75 | |
| INFL343 | Heavy chain variable | CR6323 | US20090311265, U.S. Pat. No. 8,691,223, | 23840 |
| | region | | U.S. Pat. No. 9,109,017, WO2008028946 SEQ ID | |
| | | | NO: 77 | |
| INFL344 | Heavy chain variable | CR6325 | US20090311265, U.S. Pat. No. 8,691,223, | 23841 |
| | region | | U.S. Pat. No. 9,109,017, WO2008028946 SEQ ID | |
| | | | NO: 79 | |
| INFL345 | Heavy chain variable | CR6331 | US20090311265, U.S. Pat. No. 8,691,223, | 23842 |
| | region | | U.S. Pat. No. 9,109,017, WO2008028946 SEQ ID | |
| | | | NO: 81 | |
| INFL346 | Heavy chain variable | CR6344 | US20090311265, U.S. Pat. No. 8,691,223, | 23843 |
| | region | | U.S. Pat. No. 9,109,017, WO2008028946 SEQ ID | |
| | | | NO: 83 | |
| INFL347 | Heavy chain variable | CR6141 | US20090311265, U.S. Pat. No. 8,691,223, | 23844 |
| | region | | U.S. Pat. No. 9,109,017, WO2008028946 SEQ ID | |
| | | | NO: 317 | |
| INFL348 | Heavy chain variable | CR6272 | US20090311265, U.S. Pat. No. 8,691,223, | 23845 |
| | region | | U.S. Pat. No. 9,109,017, WO2008028946 SEQ ID | |
| | | | NO: 321 | |
| INFL349 | Heavy chain variable | CR6296 | US20090311265, U.S. Pat. No. 8,691,223, | 23846 |
| | region | | U.S. Pat. No. 9,109,017, WO2008028946 SEQ ID | |
| | | | NO: 325 | |
| INFL350 | Heavy chain variable | CR6301 | US20090311265, U.S. Pat. No. 8,691,223, | 23847 |
| | region | | U.S. Pat. No. 9,109,017, WO2008028946 SEQ ID | |
| | | | NO: 329 | |
| INFL351 | Heavy chain variable | CR6327 | US20090311265, U.S. Pat. No. 8,691,223, | 23848 |
| | region | | U.S. Pat. No. 9,109,017, WO2008028946 SEQ ID | |
| | | | NO: 333 | |
| INFL352 | Heavy chain variable | CR6328 | US20090311265, U.S. Pat. No. 8,691,223, | 23849 |
| | region | | U.S. Pat. No. 9,109,017, WO2008028946 SEQ ID | |
| | | | NO: 337 | |
| INFL353 | Heavy chain variable | CR6329 | US20090311265, U.S. Pat. No. 8,691,223, | 23850 |
| | region | | U.S. Pat. No. 9,109,017, WO2008028946 SEQ ID | |
| | | | NO: 341 | |
| INFL354 | Heavy chain variable | CR6332 | US20090311265, U.S. Pat. No. 8,691,223, | 23851 |
| | region | | U.S. Pat. No. 9,109,017, WO2008028946 SEQ ID | |
| | | | NO: 345 | |
| INFL355 | Heavy chain variable | CR6334 | US20090311265, U.S. Pat. No. 8,691,223, | 23852 |
| | region | | U.S. Pat. No. 9,109,017, WO2008028946 SEQ ID | |
| | | | NO: 349 | |
| INFL356 | Heavy chain variable | CR6336 | US20090311265, U.S. Pat. No. 8,691,223, | 23853 |
| | region | | U.S. Pat. No. 9,109,017, WO2008028946 SEQ ID | |
| | | | NO: 353 | |
| INFL357 | Heavy chain variable | CR6339 | US20090311265, U.S. Pat. No. 8,691,223, | 23854 |
| | region | | U.S. Pat. No. 9,109,017, WO2008028946 SEQ ID | |
| | | | NO: 357 | |
| INFL358 | Heavy chain variable | CR6342 | US20090311265, U.S. Pat. No. 8,691,223, | 23855 |
| | region | | U.S. Pat. No. 9,109,017, WO2008028946 SEQ ID | |
| | | | NO: 361 | |
| INFL359 | Heavy chain variable | CR6343 | US20090311265, U.S. Pat. No. 8,691,223, | 23856 |
| | region | | U.S. Pat. No. 9,109,017, WO2008028946 SEQ ID | |
| | | | NO: 365 | |
| INFL360 | Heavy chain variable | CR9003 | US20140120113 SEQ ID NO: 2 | 23857 |
| | region | | | |
| INFL361 | Heavy chain variable | CR9004 | US20140120113 SEQ ID NO: 6 | 23858 |
| | region | | | |
| INFL362 | Heavy chain variable | CR9005 | US20140120113 SEQ ID NO: 10 | 23859 |
| | region | | | |
| INFL363 | Heavy chain variable | CR9006 | US20140120113 SEQ ID NO: 14 | 23860 |
| | region | | | |
| INFL364 | Heavy chain variable | CR9007 | US20140120113 SEQ ID NO: 18 | 23861 |
| | region | | | |
| INFL365 | Heavy chain variable | CR9008 | US20140120113 SEQ ID NO: 22 | 23862 |
| | region | | | |
| INFL366 | Heavy chain variable | CR9009 | US20140120113 SEQ ID NO: 26 | 23863 |
| | region | | | |
| INFL367 | Heavy chain variable | CR9010 | US20140120113 SEQ ID NO: 30 | 23864 |
| | region | | | |
| INFL368 | Heavy chain variable | CR9011 | US20140120113 SEQ ID NO: 34 | 23865 |
| | region | | | |
| INFL369 | Heavy chain variable | CR9012 | US20140120113 SEQ ID NO: 38 | 23866 |
| | region | | | |
| INFL370 | Heavy chain variable | CR9029 | US20140120113 SEQ ID NO: 42 | 23867 |
| | region | | | |
| INFL371 | Heavy chain variable | CR9030 | US20140120113 SEQ ID NO: 46 | 23868 |
| | region | | | |
| INFL372 | Heavy chain variable | CR9031 | US20140120113 SEQ ID NO: 50 | 23869 |
| | region | | | |
| INFL373 | Heavy chain variable | CR9112 | US20140120113 SEQ ID NO: 54 | 23870 |
| | region | | | |
| INFL374 | Heavy chain variable | CR9113 | US20140120113 SEQ ID NO: 58 | 23871 |
| | region | | | |
| INFL375 | Heavy chain variable | CR9114 | US20140120113 SEQ ID NO: 62 | 23872 |
| | region | | | |
| INFL376 | Heavy chain variable | CR8033 | U.S. Pat. No. 8,852,595 SEQ ID NO: 71 | 23873 |
| | region | | | |
| INFL377 | Heavy chain variable | CR8059 | U.S. Pat. No. 8,852,595 SEQ ID NO: 75 | 23874 |
| | region | | | |
| INFL378 | Heavy chain variable | CR8071 | U.S. Pat. No. 8,852,595 SEQ ID NO: 78 | 23875 |
| | region | | | |
| INFL379 | Heavy chain variable | CR10051 | U.S. Pat. No. 8,852,595 SEQ ID NO: 81 | 23876 |
| | region | | | |
| INFL380 | Heavy chain variable | CR10049 | U.S. Pat. No. 8,852,595 SEQ ID NO: 85 | 23877 |
| | region | | | |
| INFL381 | Heavy chain variable | CR10023 | U.S. Pat. No. 8,852,595 SEQ ID NO: 89 | 23878 |
| | region | | | |
| INFL382 | Heavy chain variable | CR10032 | U.S. Pat. No. 8,852,595 SEQ ID NO: 93 | 23879 |
| | region | | | |
| INFL383 | Heavy chain variable | CR11035 | U.S. Pat. No. 8,852,595 SEQ ID NO: 101 | 23880 |
| | region | | | |
| INFL384 | Heavy chain variable | CR11036 | U.S. Pat. No. 8,852,595 SEQ ID NO: 105 | 23881 |
| | region | | | |
| INFL385 | Heavy chain variable | CR11038 | U.S. Pat. No. 8,852,595 SEQ ID NO: 109 | 23882 |
| | region | | | |
| INFL386 | Heavy chain variable | CR11039 | U.S. Pat. No. 8,852,595 SEQ ID NO: 113 | 23883 |
| | region | | | |
| INFL387 | Heavy chain variable | CR8031 | U.S. Pat. No. 8,852,595 SEQ ID NO: 119 | 23884 |
| | region | | | |
| INFL388 | Heavy chain variable | CR8032 | U.S. Pat. No. 8,852,595 SEQ ID NO: 123 | 23885 |
| | region | | | |
| INFL389 | Heavy chain variable | CR8034 | U.S. Pat. No. 8,852,595 SEQ ID NO: 127 | 23886 |
| | region | | | |
| INFL390 | Heavy chain variable | CR8035 | U.S. Pat. No. 8,852,595 SEQ ID NO: 131 | 23887 |
| | region | | | |
| INFL391 | Heavy chain variable | | U.S. Pat. No. 8,992,929 SEQ ID NO: 4 | 23888 |
| | region | | | |
| INFL392 | Heavy chain variable | M2e | U.S. Pat. No. 8,420,794 SEQ ID NO: 2 | 23889 |
| | region | | | |
| INFL393 | Heavy chain variable | | U.S. Pat. No. 8,715,743, US20140275492 SEQ ID | 23890 |
| | region | | NO: 22 | |
| INFL394 | Heavy chain variable | | U.S. Pat. No. 8,715,743, US20140275492 SEQ ID | 23891 |
| | region | | NO: 25 | |
| INFL395 | Heavy chain variable | | U.S. Pat. No. 8,715,743, US20140275492 SEQ ID | 23892 |
| | region | | NO: 36 | |
| INFL396 | Heavy chain variable | 4A10 | Krause, J. C. et al. “Epitope-specific | 23893 |
| | region | | human influenza antibody repertoires | |
| | | | diversify by B cell intraclonal sequence | |
| | | | divergence and interclonal convergence” | |
| | | | J. Immunol. 187 (7), 3704-3711 (2011), | |
| | | | NCBI Accession #AEO16797 | |
| INFL397 | Heavy chain variable | anti-1918 influenza | Yu, X., et al., Neutralizing antibodies | 23894 |
| | region | HA Ig | derived front the B cells of 1918 | |
| | | | influenza pandemic survivors; Nature | |
| | | | 455 (7212), 532-536 (2008), NCBI | |
| | | | Accession #ACI04579.1 (129aa) | |
| INFL398 | Heavy chain variable | TCN-522 | US20150086555 SEQ ID NO: 33 | 23895 |
| | region | (3212_I12) | | |
| INFL399 | Heavy chain variable | TCN-521 | US20150086555 SEQ ID NO: 21 | 23896 |
| | region | (3280_D18) | | |
| INFL400 | Heavy chain variable | TCN-523 | US20150086555 SEQ ID NO: 45 | 23897 |
| | region | (5248_A17) | | |
| INFL401 | Heavy chain variable | TCN-563 | US20150086555 SEQ ID NO: 57 | 23898 |
| | region | (5237_B21) | | |
| INFL402 | Heavy chain variable | TCN-526 | US20150086555 SEQ ID NO: 69 | 23899 |
| | region | (5084_C17) | | |
| INFL403 | Heavy chain variable | TCN-527 | US20150086555 SEQ ID NO: 81 | 23900 |
| | region | (5086_C06) | | |
| INFL404 | Heavy chain variable | TCN-528 | US20150086555 SEQ ID NO: 93 | 23901 |
| | region | (5087_P17) | | |
| INFL405 | Heavy chain variable | TCN-529 | US20150086555 SEQ ID NO: 105 | 23902 |
| | region | (5297_H01) | | |
| INFL406 | Heavy chain variable | TCN-530 | US20150086555 SEQ ID NO: 117 | 23903 |
| | region | (5248_H10) | | |
| INFL407 | Heavy chain variable | TCN-531 | US20150086555 SEQ ID NO: 129 | 23904 |
| | region | (5091_H13) | | |
| INFL408 | Heavy chain variable | TCN-532 | US20150086555 SEQ ID NO: 141 | 23905 |
| | region | (5262_H18) | | |
| INFL409 | Heavy chain variable | TCN-533 | US20150086555 SEQ ID NO: 153 | 23906 |
| | region | (5256_A17a), | | |
| | | TCN-564 | | |
| | | (5256_A17b) | | |
| INFL410 | Heavy chain variable | TCN-534 | US20150086555 SEQ ID NO: 161 | 23907 |
| | region | (5249_B02) | | |
| INFL411 | Heavy chain variable | TCN-535 | US20150086555 SEQ ID NO: 173 | 23908 |
| | region | (5246_P19), | | |
| | | TCN-558 | | |
| | | (5248_H10b) | | |
| INFL412 | Heavy chain variable | TCN-536 | US20150086555 SEQ ID NO: 184 | 23909 |
| | region | (5095_N01) | | |
| INFL413 | Heavy chain variable | TCN-537 | US20150086555 SEQ ID NO: 195 | 23910 |
| | region | (3194_D21) | | |
| INFL414 | Heavy chain variable | TCN-538 | US20150086555 SEQ ID NO: 207 | 23911 |
| | region | (3206_O17) | | |
| INFL415 | Heavy chain variable | TCN-539 | US20150086555 SEQ ID NO: 219 | 23912 |
| | region | (5056_A08) | | |
| INFL416 | Heavy chain variable | TCN-540 | US20150086555 SEQ ID NO: 231 | 23913 |
| | region | (5060_F05) | | |
| INFL417 | Heavy chain variable | TCN-541 | US20150086555 SEQ ID NO: 243 | 23914 |
| | region | (5062_M11) | | |
| INFL418 | Heavy chain variable | TCN-542 | US20150086555 SEQ ID NO: 255 | 23915 |
| | region | (5079_A16) | | |
| INFL419 | Heavy chain variable | TCN-543 | US20150086555 SEQ ID NO: 267 | 23916 |
| | region | (5081_G23) | | |
| INFL420 | Heavy chain variable | TCN-544 | US20150086555 SEQ ID NO: 279 | 23917 |
| | region | (5082_A19) | | |
| INFL421 | Heavy chain variable | TCN-545 | US20150086555 SEQ ID NO: 291 | 23918 |
| | region | (5082_I15) | | |
| INFL422 | Heavy chain variable | TCN-546 | US20150086555 SEQ ID NO: 302 | 23919 |
| | region | (5089_L08) | | |
| INFL423 | Heavy chain variable | TCN-547 | US20150086555 SEQ ID NO: 313 | 23920 |
| | region | (5092_F11) | | |
| INFL424 | Heavy chain variable | TCN-548 | US20150086555 SEQ ID NO: 325 | 23921 |
| | region | (5092_P01) | | |
| INFL425 | Heavy chain variable | TCN-549 | US20150086555 SEQ ID NO: 335 | 23922 |
| | region | (5092_P04) | | |
| INFL426 | Heavy chain variable | TCN-550 | US20150086555 SEQ ID NO: 346 | 23923 |
| | region | (5096_F06) | | |
| INFL427 | Heavy chain variable | TCN-551 | US20150086555 SEQ ID NO: 358 | 23924 |
| | region | (5243_D01) | | |
| INFL428 | Heavy chain variable | TCN-552 | US20150086555 SEQ ID NO: 370 | 23925 |
| | region | (5249_I23) | | |
| INFL429 | Heavy chain variable | TCN-553 | US20150086555 SEQ ID NO: 382 | 23926 |
| | region | (5261_C18) | | |
| INFL430 | Heavy chain variable | TCN-554 | US20150086555 SEQ ID NO: 392 | 23927 |
| | region | (5277_M05) | | |
| INFL431 | Heavy chain variable | TCN-555 | US20150086555 SEQ ID NO: 403 | 23928 |
| | region | (5246_L16) | | |
| INFL432 | Heavy chain variable | TCN-556 | US20150086555 SEQ ID NO: 408 | 23929 |
| | region | (5089_K12) | | |
| INFL433 | Heavy chain variable | TCN-557 | US20150086555 SEQ ID NO: 420 | 23930 |
| | region | (5081_A04) | | |
| INFL434 | Heavy chain variable | TCN-559 | US20150086555 SEQ ID NO: 434 | 23931 |
| | region | (5097_G08) | | |
| INFL435 | Heavy chain variable | TCN-560 | US20150086555 SEQ ID NO: 446 | 23932 |
| | region | (5084_P10) | | |
| INFL436 | Heavy chain variable | TCN-504 | US20150086555 SEQ ID NO: 510 | 23933 |
| | region | (3251_K17) | | |
| INFL437 | Heavy chain variable | AB1 | US20120093834, WO2009121004 SEQ | 23934 |
| | region | | ID NO: 4 | |
| INFL438 | Heavy chain variable | AB2 | US20120093834, WO2009121004 SEQ | 23935 |
| | region | | ID NO: 45 | |
| INFL439 | Heavy chain variable | AB3 | US20120093834, WO2009121004 SEQ | 23936 |
| | region | | ID NO: 9 | |
| INFL440 | Heavy chain variable | AB4, AB5, AB6 | US20120093834, WO2009121004 SEQ | 23937 |
| | region | | ID NO: 61 | |
| INFL441 | Heavy chain variable | VN04-2 | WO2008033105 SEQ ID NO: 5 | 23938 |
| | region | | | |
| INFL442 | Heavy chain variable | VN04-3 | WO2008033105 SEQ ID NO: 7 | 23939 |
| | region | | | |
| INFL443 | Heavy chain variable | 1286-C05 | WO2010132604, US20120128671 SEQ | 23940 |
| | region | | ID NO: 1 | |
| INFL444 | Heavy chain variable | 1286-A11 | WO2010132604, US20120128671 SEQ | 23941 |
| | region | | ID NO: 2 | |
| INFL445 | Heavy chain variable | CR8001 | WO2010130636 SEQ ID NO: 2 | 23942 |
| | region | | | |
| INFL446 | Heavy chain variable | CR8003 | WO2010130636 SEQ ID NO: 6 | 23943 |
| | region | | | |
| INFL447 | Heavy chain variable | CR8015 | WO2010130636 SEQ ID NO: 10 | 23944 |
| | region | | | |
| INFL448 | Heavy chain variable | CR8016 | WO2010130636 SEQ ID NO: 14 | 23945 |
| | region | | | |
| INFL449 | Heavy chain variable | CR8017 | WO2010130636 SEQ ID NO: 18 | 23946 |
| | region | | | |
| INFL450 | Heavy chain variable | CR8018 | WO2010130636 SEQ ID NO: 22 | 23947 |
| | region | | | |
| INFL451 | Heavy chain variable | anti-1918 influenza | Yu, X., et al., Neutralizing antibodies | 23948 |
| | region (Partial) | HA Ig | derived from the B cells of 1918 | |
| | | | influenza pandemic survivors; Nature | |
| | | | 455 (7212), 532-536 (2008), NCBI | 23949 |
| | | | Accession #ACI04581.1 (145aa) | |
| INFL452 | Heavy chain variable | 1A2 | WO2015028478 SEQ ID NO: 2 | 23950 |
| | region mouse IgG | | | |
| INFL453 | Heavy chain variable | 7B8 | WO2015028478 SEQ ID NO: 4 | 23951 |
| | region mouse IgG | | | |
| INFL454 | Heavy chain variable | monoclonal | Grandea, A. G. et al., Human antibodies | 23952 |
| | region, partial | antibody TCN-031 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23854.1 | |
| | | | (120aa) | |
| INFL455 | Heavy chain variable | monoclonal | Grandea, A. G. et al., Human, antibodies | 23953 |
| | region, partial | antibody TCN-032 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23853.1 | |
| | | | (120aa) | |
| INFL456 | Heavy chain variable | monoclonal | Grandea, A. G. et al., Human antibodies | 23954 |
| | region, partial | antibody 3362_B11 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23869.1 | |
| | | | (123aa) | |
| INFL457 | Heavy chain variable | monoclonal | Grandea, A. G. et al., Human antibodies | 23955 |
| | region, partial | antibody | reveal a protective epitope that is highly | |
| | | 3260_D19 | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23868.1 | |
| | | | (118aa) | |
| INFL458 | Heavy chain variable | monoclonal | Grandea, A. G. et al., Human antibodies | 23956 |
| | region, partial | antibody 3253_P10 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23867.1 | |
| | | | (121aa) | |
| INFL459 | Heavy chain variable | monoclonal | Grandea, A. G. et al., Human antibodies | 23957 |
| | region, partial | antibody 3248_P18 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23866.1 | |
| | | | (120aa) | |
| INFL460 | Heavy chain variable | monoclonal | Grandea, A. G. et al., Human antibodies | 23958 |
| | region, partial | antibody 3139_P23 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23865.1 | |
| | | | (119aa) | |
| INFL461 | Heavy chain variable | monoclonal | Grandea, A. G. et al., Human antibodies | 23959 |
| | region, partial | antibody 3420_I23 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23864.1 | 23960 |
| | | | (121aa) | |
| INFL462 | Heavy chain variable | monoclonal | Grandea, A. G. et al., Human antibodies | 23961 |
| | region, partial | antibody 3255_J06 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23863.1 | |
| | | | (119aa) | |
| INFL463 | Heavy chain variable | monoclonal | Grandea, A. G. et al., Human antibodies | 23962 |
| | region, partial | antibody 3252_C13 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession # ADK23862.1 | |
| | | | (119aa) | |
| INFL464 | Heavy chain variable | monoclonal | Grandea, A. G. et al., Human antibodies | 23963 |
| | region, partial | antibody | reveal a protective epitope that is highly | |
| | | 3136_G05 | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23861.1 | |
| | | | (120aa) | |
| INFL465 | Heavy chain variable | monoclonal | Grandea, A. G. et al., Human antibodies | 23964 |
| | region, partial | antibody | reveal a protective epitope that is highly | |
| | | 3244_H04 | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23860.1 | |
| | | | (118aa) | |
| INFL466 | Heavy chain variable | monoclonal | Grandea, A. G. et al., Human antibodies | 23965 |
| | region, partial | antibody | reveal a protective epitope that is highly | |
| | | 3245_O19 | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23859.1 | |
| | | | (118aa) | |
| INFL467 | Heavy chain variable | monoclonal | Grandea, A. G. et al., Human antibodies | 23966 |
| | region, partial | antibody 3259_J21 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23858.1 | |
| | | | (120aa) | |
| INFL468 | Heavy chain variable | monoclonal | Grandea, A. G. et al., Human antibodies | 23967 |
| | region, partial | antibody 3243_J07 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23857.1 | |
| | | | (121aa) | |
| INFL469 | Heavy chain variable | monoclonal | Grandea, A. G. et al., Human antibodies | 23968 |
| | region, partial | antibody 3244_I10 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession # ADK23856.1 | |
| | | | (121aa) | |
| INFL470 | Heavy chain variable | monoclonal | Grandea, A. G. et al., Human antibodies | 23969 |
| | region, partial | antibody | reveal a protective epitope that is highly | |
| | | 3241_G23 | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23855.1 | |
| | | | (122aa) | |
| INFL471 | Heavy chain variable | Monoclonal | Yasugi, M. et al., Emerging Antigenic | 23970 |
| | region, partial | antibody clone 5E4 | Variants at the Antigenic Site Sb in | |
| | | | Pandemic A(H1N1)2009 Influenza Virus | |
| | | | in Japan Detected by a Human | |
| | | | Monoclonal Antibody; PLoS ONE 8 | |
| | | | (10), E77892 (2013), NCBI Accession | |
| | | | #BAM76754.1 (141aa) | |
| INFL472 | Heavy chain variable | 100F4-HV | Hu, H., et al., A Human Antibody | 23971 |
| | region, partial | | Recognizing a Conserved Epitope of H5 | |
| | | | Hemagglutinin Broadly Neutralizes | |
| | | | Highly Pathogenic Avian Influenza | |
| | | | H5N1 Viruses; J. Virol. 86 (6), 2978- | |
| | | | 2989 (2012), NCBI Accession | |
| | | | #AEL30603.1 (121aa) | |
| INFL473 | Heavy chain variable | anti-stem HA | Pappas, L. et al., Rapid development of | 23972 |
| | region, partial, Anti- | immunoglobulin | broadly influenza neutralizing antibodies | |
| | stem HA | | through redundant mutations; Nature | |
| | immunoglobulin | | (2014), NCBI Accession #AIN40460.1 | |
| | | | (120aa) | |
| INFL474 | Heavy chain variable | anti-stem HA | Pappas, L. et al., Rapid development of | 23973 |
| | region, partial, Anti- | immunoglobulin | broadly influenza neutralizing antibodies | |
| | stem HA | | through redundant mutations; Nature | |
| | immunoglobulin | | (2014), NCBI Accession #AIN40459.1 | |
| | | | (127aa) | |
| INFL475 | Heavy chain variable | anti-stem HA | Pappas, L. et al., Rapid development of | 23974 |
| | region, partial, Anti- | immunoglobulin | broadly influenza neutralizing antibodies | |
| | stem HA | | through redundant mutations; Nature | |
| | immunoglobulin | | (2014), NCBI Accession #AIN40458.1 | |
| | | | (129aa) | |
| INFL476 | Heavy chain variable | anti-stem HA | Pappas, L. et al., Rapid development of | 23975 |
| | region, partial, Anti- | immunoglobulin | broadly influenza neutralizing antibodies | |
| | stem HA | | through redundant mutations; Nature | |
| | immunoglobulin | | (2014), NCBI Accession #AIN40457.1 | |
| | | | (132aa) | |
| INFL477 | Heavy chain variable | anti-stem HA | Pappas, L. et al., Rapid development of | 23976 |
| | region, partial, Anti- | immunoglobulin | broadly influenza neutralizing antibodies | |
| | stem HA | | through redundant mutations; Nature | |
| | immunoglobulin | | (2014), NCBI Accession #AIN40456.1 | |
| | | | (127aa) | |
| INFL478 | Heavy chain variable | anti-stem HA | Pappas, L. et al., Rapid development of | 23977 |
| | region, partial, Anti- | immunoglobulin | broadly influenza neutralizing antibodies | |
| | stem HA | | through redundant mutations; Nature | |
| | immunoglobulin | | (2014), NCBI Accession #AIN40455.1 | |
| | | | (121aa) | |
| INFL479 | Heavy chain variable | anti-stem HA | Pappas, L. et al., Rapid development of | 23978 |
| | region, partial, Anti- | immunoglobulin | broadly influenza neutralizing antibodies | |
| | stem HA | | through redundant mutations; Nature | |
| | immunoglobulin | | (2014), NCBI Accession #AIN40454.1 | |
| | | | (126aa) | |
| INFL480 | Heavy chain variable | anti-stem HA | Pappas, L. et al., Rapid development of | 23979 |
| | region, partial, Anti- | immunoglobulin | broadly influenza neutralizing antibodies | |
| | stem HA | | through redundant mutations; Nature | |
| | immunoglobulin | | (2014), NCBI Accession #AIN40453.1 | 23980 |
| | | | (120aa) | |
| INFL481 | Heavy chain variable | anti-stem HA | Pappas, L. et al., Rapid development of | 23981 |
| | region, partial, Anti- | immunoglobulin | broadly influenza neutralizing antibodies | |
| | stem HA | | through redundant mutations; Nature | |
| | immunoglobulin | | (2014), NCBI Accession #AIN40452.1 | |
| | | | (122aa) | |
| INFL482 | Heavy chain variable | anti-stem HA | Pappas, L. et al., Rapid development of | 23982 |
| | region, partial, Anti- | immunoglobulin | broadly influenza neutralizing antibodies | |
| | stem HA | | through redundant mutations; Nature | |
| | immunoglobulin | | (2014), NCBI Accession #AIN40451.1 | |
| | | | (125aa) | |
| INFL483 | Heavy chain variable | anti-stem HA | Pappas, L. et al., Rapid development of | 23983 |
| | region, partial, Anti- | immunoglobulin | broadly influenza neutralizing antibodies | |
| | stem HA | | through redundant mutations; Nature | |
| | immunoglobulin | | (2014), NCBI Accession #AIN40450.1 | |
| | | | (126aa) | |
| INFL484 | Heavy chain variable | anti-stem HA | Pappas, L. et al., Rapid development of | 23984 |
| | region, partial, Anti- | immunoglobulin | broadly influenza neutralizing antibodies | |
| | stem HA | | through redundant mutations; Nature | |
| | immunoglobulin | | (2014), NCBI Accession #AIN40449.1 | |
| | | | (129aa) | |
| INFL485 | Heavy chain variable | anti-stem HA | Pappas, L. et al., Rapid development of | 23985 |
| | region, partial, Anti- | immunoglobulin | broadly influenza neutralizing antibodies | |
| | stem HA | | through redundant mutations; Nature | |
| | immunoglobulin | | (2014), NCBI Accession #AIN40448.1 | |
| | | | (119aa) | |
| INFL486 | Heavy chain variable | anti-stem HA | Pappas, L. et al., Rapid development of | 23986 |
| | region, partial, Anti- | immunoglobulin | broadly influenza neutralizing antibodies | |
| | stem HA | | through redundant mutations; Nature | |
| | immunoglobulin | | (2014), NCBI Accession #AIN40447.1 | |
| | | | (120aa) | |
| INFL487 | Heavy chain variable | anti-stem HA | Pappas, L. et al., Rapid development of | 23987 |
| | region, partial, Anti- | immunoglobulin | broadly influenza neutralizing antibodies | |
| | stem HA | | through redundant mutations; Nature | |
| | immunoglobulin | | (2014), NCBI Accession #AIN40446.1 | |
| | | | (120aa) | |
| INFL488 | Heavy chain, Human | Fab 39.29 | Nakamura, G. et al., An in vivo human- | 23988 |
| | igg, | | plasmablast enrichment technique allows | |
| | | | rapid identification of therapeutic | |
| | | | influenza a antibodies; Cell Host | |
| | | | Microbe 14 (1), 93-103 (2013), NCBI | |
| | | | Accession #4KVN_H (227aa) | |
| INFL489 | Heavy chain, Igg1 | Fab H5m9 | Zhu, X., et al., A Unique and Conserved | 23989 |
| | | | Neutralization Epitope in H5N1 | |
| | | | Influenza Viruses Identified by an | |
| | | | Antibody against the | |
| | | | A/Goose/Guangdong/1/96 | |
| | | | Hemagglutinin; J. Virol. 87 (23), 12619- | |
| | | | 12635 (2013), NCBI Accession | |
| | | | #4MHH_H (222aa) | |
| INFL490 | Immunoglobulin | T2-6A | Huang, K.-Y. A., et al., Focused antibody- | 23990 |
| | heavy chain variable | | response to influenza linked to antigenic | |
| | region, partial | | drift; J. Clin. Invest. (2015), NCBI | |
| | | | Accession #AKF02484.1 (124aa) | |
| INFL491 | Kappa light chain | | U.S. Pat. No. 8,992,929 SEQ ID NO. 24 | 23991 |
| | constant region, | | | |
| | human | | | |
| INFL492 | Kappa light chain | 8D4 | NCBI Accession #AFI57037 | 23992 |
| | variable | | | |
| INFL493 | Kappa light chain | 5B6 | NCBI Accession #AFI57041 | 23993 |
| | variable | | | |
| INFL494 | Kappa light chain | 8i10 | U.S. Pat. No. 8,858,948 SEQ ID NO: 56 | 23994 |
| | variable region | | | |
| INFL495 | Kappa light chain | 23K12 | U.S. Pat. No. 8,858,948 SEQ ID NO: 91 | 23995 |
| | variable region | | | |
| INFL496 | Kappa light chain | 4K8 | Krause, J. C. et al. “Epitope-specific | 23996 |
| | variable region | | human influenza antibody repertoires | |
| | | | diversify by B cell intraclonal sequence | |
| | | | divergence and interclonal convergence” | |
| | | | J. Immunol. 187 (7), 3704-3711 (2011), | |
| | | | NCBI Accession #AEO16800 | |
| INFL497 | Kappa light chain | 6D9 | Krause, J. C. et al. “Epitope-specific | 23997 |
| | variable region | | human influenza antibody repertoires | |
| | | | diversify by B cell intraclonal sequence | |
| | | | divergence and interclonal convergence” | |
| | | | J. Immunol. 187 (7), 3704-3711 (2011), | |
| | | | NCBI Accession #AEO16802 | |
| INFL498 | Kappa light chain | 8G9 | U.S. Pat. No. 8,603,467 SEQ ID NO: 4 | 23998 |
| | variable region | | | |
| INFL499 | Kappa light chain | 13D4 | U.S. Pat. No. 8,603,467 SEQ ID NO: 8 | 23999 |
| | variable region | | | |
| INFL500 | Kappa light chain | 20A11 | U.S. Pat. No. 8,603,467 SEQ ID NO: 12 | 24000 |
| | variable region | | | |
| INFL501 | Kappa light chain | EM4C04 | US20120282273 SEQ ID NO: 1 | 24001 |
| | variable region | | | |
| INFL502 | Kappa, light chain | Fab H5m9 | Zhu, X., et al., A Unique and Conserved | 24002 |
| | | | Neutralization Epitope in H5N1 | |
| | | | Influenza Viruses Identified by an | |
| | | | Antibody against the | |
| | | | A/Goose/Guangdong/1/96 | |
| | | | Hemagglutinin; J. Virol. 87 (23), 12619- | |
| | | | 12635 (2013), NCBI Accession # | |
| | | | 4MHH_L(218aa) | |
| INFL503 | Lambda light chain | 7A13 | Krause et al. “Human Monoclonal | 24003 |
| | | | Antibodies to Pandemic 1957 H2N2 and | |
| | | | Pandemic 1968 H3N2 Influenza Viruses” | |
| | | | J. Virol. 86 (11), 6334-6340 (2012), | |
| | | | NCBI Accession #AFH78448 | |
| INFL504 | Lambda light chain | 2K11 | Krause, J. C. et al. “Epitope-specific | 24004 |
| | variable | | human influenza antibody repertoires | |
| | | | diversify by B cell intraclonal sequence | |
| | | | divergence and interclonal convergence” | |
| | | | J. Immunol. 187 (7), 3704-3711 (2011), | |
| | | | NCBI Accession #AEO16794 | |
| INFL505 | Lambda light chain | 2O10 | Krause, J. C. et al. “Epitope-specific | 24005 |
| | variable | | human influenza antibody repertoires | |
| | | | diversify by B cell intraclonal sequence | |
| | | | divergence and interclonal convergence” | |
| | | | J. Immunol. 187 (7), 3704-3711 (2011), | |
| | | | NCBI Accession #AEO16796 | |
| INFL506 | Lambda light chain | Monoclonal | Yasugi, M. et al., Emerging Antigenic | 24006 |
| | variable region, | antibody clone 5E4 | Variants at the Antigenic Site Sb in | |
| | partial | | Pandemic A(H1N1)2009 Influenza Virus | |
| | | | in Japan Detected by a Human | |
| | | | Monoclonal Antibody; PLoS ONE 8 | |
| | | | (10), E77892 (2013), NCBI Accession | |
| | | | #BAM76755.1 (126aa) | |
| INFL507 | Lambda light chain | T2-6A | Huang, K.-Y. A., et al., Focused antibody | 24007 |
| | variable region, | | response to influenza linked to antigenic | |
| | partial, | | drift; J. Clin. Invest. (2015), NCBI | 24008 |
| | Immunoglobulin | | Accession #AKF02488.1 (113aa) | |
| INFL508 | Light chain | CR6261, | WO2008028946 | 24009 |
| | | Diridavumab, CR- | | |
| | | 6261 | | |
| INFL509 | Light chain | Firivumab, CT-P22 | US20130004505 | 24010 |
| INFL510 | Light chain | Navivumab, | WO2013048153, US20140234336 SEQ | 24011 |
| | | CT149 | ID NO: 39 | |
| INFL511 | Light chain | AT10-004 | US20150010566, WO2013081463 SEQ | 24012 |
| | | | ID NO: 36 | |
| INFL512 | Light chain | AT10-003 | US20150010566, WO2013081463 SEQ | 24013 |
| | | | ID NO: 37 | |
| INFL513 | Light chain | AT10-002 | US20150010566, WO2013081463 SEQ | 24014 |
| | | | ID NO: 38 | |
| INFL514 | Light chain | AT10-001 | US20150010566, WO2013081463 SEQ | 24015 |
| | | | ID NO: 39 | |
| INFL515 | Light chain | AT10-005 | US20150010566, WO2013081463 SEQ | 24016 |
| | | | ID NO: 40 | |
| INFL516 | Light chain | CT104 | WO2011111966, US20130004505 SEQ | 24017 |
| | | | ID NO: 36 | |
| INFL517 | Light chain | CT120 | WO2011111966, US20130004505 SEQ | 24018 |
| | | | ID NO: 40 | |
| INFL518 | Light chain | CT123 | WO2011111966, US20130004505 SEQ | 24019 |
| | | | ID NO: 44 | |
| INFL519 | Light chain | 2A | US20140011982 SEQ ID NO: 4 | 24020 |
| INFL520 | Light chain | F005-126 | WO2014049520, US20140086927 SEQ | 24021 |
| | | | ID NO: 13 | |
| INFL521 | Light chain | BF1-1 | WO2008156763 SEQ ID NO: 8 | 24022 |
| INFL522 | Light chain | BF1-19 | WO2008156763 SEQ ID NO: 12 | 24023 |
| INFL523 | Light chain | BF1-10 | WO2008156763 SEQ ID NO: 10 | 24024 |
| INFL524 | Light chain | 2D1 | WO2010127252, U.S. Pat. No. 8,894,997 SEQ ID | 24025 |
| | | | NO: 8 | |
| INFL525 | Light chain | 1F1 | WO2010127252, U.S. Pat. No. 8,894,997 SEQ ID | 24026 |
| | | | NO: 2 | |
| INFL526 | Light chain | 4D20 | WO2010127252, U.S. Pat. No. 8,894,997 SEQ ID | 24027 |
| | | | NO: 10 | |
| INFL527 | Light chain | A18 | WO13170139 SEQ ID NO: 95 | 24028 |
| INFL528 | Light chain | Ab A18 | U.S. Pat. No. 7,788,200 SEQ ID NO: 28 | 24029 |
| INFL529 | Light chain | Ab 067 | U.S. Pat. No. 7,788,200 SEQ ID NO: 153 | 24030 |
| INFL530 | Light chain | Ab 068 | U.S. Pat. No. 7,788,200 SEQ ID NO: 154 | 24031 |
| INFL531 | Light chain | Ab 069, Ab 079 | U.S. Pat. No. 7,788,200 SEQ ID NO: 155 | 24032 |
| INFL532 | Light chain | Ab 070 | U.S. Pat. No. 7,788,200 SEQ ID NO: 156 | 24033 |
| INFL533 | Light chain | Ab 073 | U.S. Pat. No. 7,788,200 SEQ ID NO: 165 | 24034 |
| INFL534 | Light chain | Ab 074, Ab 080 | U.S. Pat. No. 7,788,200 SEQ ID NO: 166 | 24035 |
| INFL535 | Light chain | Ab 075 | U.S. Pat. No. 7,788,200 SEQ ID NO: 167 | 24036 |
| INFL536 | Light chain | Ab 076 | U.S. Pat. No. 7,788,200 SEQ ID NO: 168 | 24037 |
| INFL537 | Light chain | Ab 077, Ab 081 | U.S. Pat. No. 7,788,200 SEQ ID NO: 169 | 24038 |
| INFL538 | Light chain | Ab 014, Ab 154, | U.S. Pat. No. 7,788,200 SEQ ID NO: 29 | 24039 |
| | | Ab 157 | | |
| INFL539 | Light chain | Ab 028, Ab 155 | U.S. Pat. No. 7,788,200 SEQ ID NO: 30 | 24040 |
| INFL540 | Light chain | Ab 001, Ab 002, | U.S. Pat. No. 7,788,200 SEQ ID NO: 31 | 24041 |
| | | Ab 003 | | |
| INFL541 | Light chain | Ab 009, Ab 010, | U.S. Pat. No. 7,788,200 SEQ ID NO: 32 | 24042 |
| | | Ab 011 | | |
| INFL542 | Light chain | Ab 017, Ab B18, | U.S. Pat. No. 7,788,200 SEQ ID NO: 33 | 24043 |
| | | Ab B18, Ab 019, | | |
| | | Ab 019 | | |
| INFL543 | Light chain | Ab 025, Ab 026, | U.S. Pat. No. 7,788,200 SEQ ID NO: 34 | 24044 |
| | | Ab 027, Ab 028 | | |
| INFL544 | Light chain | Ab 159 | U.S. Pat. No. 7,788,200 SEQ ID NO: 35 | 24045 |
| INFL545 | Light chain | Ab 160 | U.S. Pat. No. 7,788,200 SEQ ID NO: 36 | 24046 |
| INFL546 | Light chain | Ab 186, Ab 194 | U.S. Pat. No. 7,788,200 SEQ ID NO: 37 | 24047 |
| INFL547 | Light chain | Ab 187, Ab 195 | U.S. Pat. No. 7,788,200 SEQ ID NO: 38 | 24048 |
| INFL548 | Light chain | Ab 188, Ab 196 | U.S. Pat. No. 7,788,200 SEQ ID NO: 39 | 24049 |
| INFL549 | Light chain | Ab 189, Ab 197 | U.S. Pat. No. 7,788,200 SEQ ID NO: 40 | 24050 |
| INFL550 | Light chain | Ab 190, Ab 198 | U.S. Pat. No. 7,788,200 SEQ ID NO: 41 | 24051 |
| INFL551 | Light chain | Ab 191, Ab 199 | U.S. Pat. No. 7,788,200 SEQ ID NO: 42 | 24052 |
| INFL552 | Light chain | Ab 192, Ab 200 | U.S. Pat. No. 7,788,200 SEQ ID NO: 43 | 24053 |
| INFL553 | Light chain | Ab 193 | U.S. Pat. No. 7,788,200 SEQ ID NO: 44 | 24054 |
| INFL554 | Light chain | Ab 202, Ab 203, | U.S. Pat. No. 7,788,200 SEQ ID NO: 45 | 24055 |
| | | Ab 204, Ab 210, | | |
| | | Ab 031, Ab 032, | | |
| | | Ab 033, Ab 034 | | |
| INFL555 | Light chain | Ab 211, Ab 212, | U.S. Pat. No. 7,788,200 SEQ ID NO: 46 | 24056 |
| | | Ab 213, Ab 219, | | |
| | | Ab 037, Ab 038, | | |
| | | Ab 039, Ab 040 | | |
| INFL556 | Light chain | Ab A001, Ab 004, | U.S. Pat. No. 7,788,200 SEQ ID NO: 47 | 24057 |
| | | Ab 007, Ab 016 | | |
| INFL557 | Light chain | Ab A002, Ab 005, | U.S. Pat. No. 7,788,200 SEQ ID NO: 48 | 24058 |
| | | Ab 008, Ab A017 | | |
| INFL558 | Light chain | Ab A003, Ab 006, | U.S. Pat. No. 7,788,200 SEQ ID NO: 49 | 24059 |
| | | Ab A009, Ab C18 | | |
| INFL559 | Light chain | Ab A010, Ab 012, | U.S. Pat. No. 7,788,200 SEQ ID NO: 50 | 24060 |
| | | Ab A14, Ab A019 | | |
| INFL560 | Light chain | Ab A011, Ab | U.S. Pat. No. 7,788,200 SEQ ID NO: 51 | 24061 |
| | | 013m Ab 0135 | | |
| INFL561 | Light chain | Ab 044, Ab 071, | U.S. Pat. No. 7,788,200 SEQ ID NO: 52 | 24062 |
| | | Ab 072, Ab 078 | | |
| INFL562 | Light chain | Ab 051 | U.S. Pat. No. 7,788,200 SEQ ID NO: 53 | 24063 |
| INFL563 | Light chain | Ab 049 | U.S. Pat. No. 7,788,200 SEQ ID NO: 54 | 24064 |
| INFL564 | Light chain | Ab 047 | U.S. Pat. No. 7,788,200 SEQ ID NO: 55 | 24065 |
| INFL565 | Light chain | Ab 050 | U.S. Pat. No. 7,788,200 SEQ ID NO: 56 | 24066 |
| INFL566 | Light chain | Ab 045 | U.S. Pat. No. 7,788,200 SEQ ID NO: 57 | 24067 |
| INFL567 | Light chain | Ab 048 | U.S. Pat. No. 7,788,200 SEQ ID NO: 58 | 24068 |
| INFL568 | Light chain | Ab 046 | U.S. Pat. No. 7,788,200 SEQ ID NO: 59 | 24069 |
| INFL569 | Light chain | Ab 043 | U.S. Pat. No. 7,788,200 SEQ ID NO: 60 | 24070 |
| INFL570 | Light chain | Ab 052 | U.S. Pat. No. 7,788,200 SEQ ID NO: 61 | 24071 |
| INFL571 | Light chain | F005-126 | WO2014049520 SEQ ID 13 | 24072 |
| INFL572 | Light chain | 8f24 | WO2012045001 SEQ ID 3 | 24073 |
| INFL573 | Light chain | 3E22 | WO2012045001 SEQ ID 7 | 24074 |
| INFL574 | Light chain | 5117 | WO2012045001 SEQ ID 11 | 24075 |
| INFL575 | Light chain | | WO2012045001 SEQ ID 15 | 24076 |
| INFL576 | Light chain | | WO2012045001 SEQ ID 31 | 24077 |
| INFL577 | Light chain | | WO2012045001 SEQ ID 35 | 24078 |
| INFL578 | Light chain | | WO2012045001 SEQ ID 19 | 24079 |
| INFL579 | Light chain | 10A14 | WO2012045001 SEQ ID 23 | 24080 |
| INFL580 | Light chain | 8D4 | WO2012045001 SEQ ID 27 | 24081 |
| INFL581 | Light chain | 2B9 | U.S. Pat. No. 9,115,201 SEQ ID NO: 5 | 24082 |
| INFL582 | Light chain | mAB 7A7 | US20150239960, US20140170163, | 24083 |
| | | | U.S. Pat. No. 8,673,314, US20110027270, | |
| | | | WO2010138564 SEQ ID NO: 7 | |
| INFL583 | Light chain | mAB 12D1 | US20150239960, US20140170163, | 24084 |
| | | | U.S. Pat. No. 8,673,314, US20110027270, | |
| | | | WO2010138564 SEQ ID NO: 13 | |
| INFL584 | Light chain | mAB 66A6 | US20150239960, US20140170163, | 24085 |
| | | | U.S. Pat. No. 8,673,314, US20110027270, | |
| | | | WO2010138564 SEQ ID NO: 17 | |
| INFL585 | Light chain | M1 D12 | US20110033473, WO2009125395 SEQ | 24086 |
| | | | ID NO: 15 | |
| INFL586 | Light chain | mAB1.12 | WO2013030165 SEQ ID NO: 2 | 24087 |
| INFL587 | Light chain | mAB3.1 | WO2013030165 SEQ ID NO: 4 | 24088 |
| INFL588 | Light chain | 5A7 | WO2015120097 SEQ ID NO: 8 | 24089 |
| INFL589 | Light chain | TRL053 | WO2015120097 SEQ ID NO: 18 | 24090 |
| INFL590 | Light chain | TRL579 | WO2015120097 SEQ ID NO: 28 | 24091 |
| INFL591 | Light chain | TRL784 | WO2015120097 SEQ ID NO: 38 | 24092 |
| INFL592 | Light chain | TRL794 | WO2015120097 SEQ ID NO: 48 | 24093 |
| INFL593 | Light chain | TRL798 | WO2015120097 SEQ ID NO: 58 | 24094 |
| INFL594 | Light chain | TRL799 | WO2015120097 SEQ ID NO: 68 | 24095 |
| INFL595 | Light chain | TRL809 | WO2015120097 SEQ ID NO: 78 | 24096 |
| INFL596 | Light chain | TRL811 | WO2015120097 SEQ ID NO: 88 | 24097 |
| INFL597 | Light chain | TRL812 | WO2015120097 SEQ ID NO: 98 | 24098 |
| INFL598 | Light chain | TRL813 | WO2015120097 SEQ ID NO: 108 | 24099 |
| INFL599 | Light chain | TRL823 | WO2015120097 SEQ ID NO: 118 | 24100 |
| INFL600 | Light chain | TRL832 | WO2015120097 SEQ ID NO: 128 | 24101 |
| INFL601 | Light chain | TRL833 | WO2015120097 SEQ ID NO: 138 | 24102 |
| INFL602 | Light chain | TRL834 | WO2015120097 SEQ ID NO: 148 | 24103 |
| INFL603 | Light chain | TRL837 | WO2015120097 SEQ ID NO: 167 | 24104 |
| INFL604 | Light chain | TRL839 | WO2015120097 SEQ ID NO: 177 | 24105 |
| INFL605 | Light chain | TRL841 | WO2015120097 SEQ ID NO: 187 | 24106 |
| INFL606 | Light chain | TRL842 | WO2015120097 SEQ ID NO: 197 | 24107 |
| INFL607 | Light chain | TRL845 | WO2015120097 SEQ ID NO: 207 | 24108 |
| INFL608 | Light chain | TRL846 | WO2015120097 SEQ ID NO: 218 | 24109 |
| INFL609 | Light chain | TRL847 | WO2015120097 SEQ ID NO: 228 | 24110 |
| INFL610 | Light chain | TRL848 | WO2015120097 SEQ ID NO: 238 | 24111 |
| INFL611 | Light chain | TRL849 | WO2015120097 SEQ ID NO: 248 | 24112 |
| INFL612 | Light chain | TRL851 | WO2015120097 SEQ ID NO: 258 | 24113 |
| INFL613 | Light chain | TRL854 | WO2015120097 SEQ ID NO: 268 | 24114 |
| INFL614 | Light chain | TRL856 | WO2015120097 SEQ ID NO: 278 | 24115 |
| INFL615 | Light chain | TRL858 | WO2015120097 SEQ ID NO: 288 | 24116 |
| INFL616 | Light chain | humM2e-hBiTE-1 | WO2014140368 SEQ ID NO: 10 | 24117 |
| INFL617 | Light chain | humM2e-hBiTE-2 | WO2014140368 SEQ ID NO: 18 | 24118 |
| INFL618 | Light chain | humM2e-hBiTE-3 | WO2014140368 SEQ ID NO: 26 | 24119 |
| INFL619 | Light chain | humM2e-hBiTE-4 | WO2014140368 SEQ ID NO: 34 | 24120 |
| INFL620 | Light chain | VH of humM2e- | WO2014140368 SEQ ID NO: 42 | 24121 |
| | | hBiTE-5 | | |
| INFL621 | Light chain | humM2e-hBiTE-6 | WO2014140368 SEQ ID NO: 50 | 24122 |
| INFL622 | Light chain | humM2e-hBiTE-7 | WO2014140368 SEQ ID NO: 58 | 24123 |
| INFL623 | Light chain | humM2e-hBiTE-8 | WO2014140368 SEQ ID NO: 66 | 24124 |
| INFL624 | Light chain | humM2e-hBiTE-9 | WO2014140368 SEQ ID NO: 74 | 24125 |
| INFL625 | Light chain | murM2e-hBiTE | WO2014140368 SEQ ID NO: 82 | 24126 |
| INFL626 | Light chain | FLA5.10 | U.S. Pat. No. 8,124,092 SEQ ID NO: 3 | 24127 |
| INFL627 | Light chain | FLD21.140 | U.S. Pat. No. 8,124,092 SEQ ID NO: 7 | 24128 |
| INFL628 | Light chain | FLA3.14 | U.S. Pat. No. 8,124,092 SEQ ID NO: 11 | 24129 |
| INFL629 | Light chain | FLD20.19 | U.S. Pat. No. 8,124,092 SEQ ID NO: 15 | 24130 |
| INFL630 | Light chain | FLD84 | U.S. Pat. No. 8,124,092 SEQ ID NO: 44 | 24131 |
| INFL631 | Light chain | FLD93 | U.S. Pat. No. 8,124,092 SEQ ID NO: 54 | 24132 |
| INFL632 | Light chain | FLD122 | U.S. Pat. No. 8,124,092 SEQ ID NO: 64 | 24133 |
| INFL633 | Light chain | FLD127 | U.S. Pat. No. 8,124,092 SEQ ID NO: 74 | 24134 |
| INFL634 | Light chain | FLD129 | U.S. Pat. No. 8,124,092 SEQ ID NO: 84 | 24135 |
| INFL635 | Light chain | FLD132 | U.S. Pat. No. 8,124,092 SEQ ID NO: 94 | 24136 |
| INFL636 | Light chain | FLD194 | U.S. Pat. No. 8,124,092 SEQ ID NO: 104 | 24137 |
| INFL637 | Light chain | mAb1 | WO2015112994 SEQ ID NO: 77 | 24138 |
| INFL638 | Light chain | mAb2 | WO2015112994 SEQ ID NO: 81 | 24139 |
| INFL639 | Light chain | mAb3 | WO2015112994 SEQ ID NO: 85 | 24140 |
| INFL640 | Light chain | CT-P22 | US20130004505 SEQ ID NO: 40; | 24141 |
| | | | WO2011/111966 | |
| INFL641 | Light chain | C05 | Ekiert, D. C., et al., Cross-neutralization | 24142 |
| | | | of influenza A viruses mediated by a | |
| | | | single antibody loop; Nature 489 (7417), | |
| | | | 526-532 (2012), NCBI Accession | |
| | | | #4FNL_L (214aa) | |
| INFL642 | Light chain | CR8020 | Ekiert, D. C., et al., A highly conserved | 24143 |
| | | | neutralizing epitope on group 2 influenza | |
| | | | A viruses; Science 333 (6044), 843-850 | |
| | | | (2011); WO2010130636, NCBI | |
| | | | Accession #3SDY_L | |
| INFL643 | Light chain | CR8033 | Dreyfus, C., Laursen, N. S. et al., Highly | 24144 |
| | | | conserved protective epitopes on | |
| | | | influenza B viruses; Science 337 (6100), | |
| | | | 1343-1348 (2012), NCBI Accession # | |
| | | | 4FQL_L | |
| INFL644 | Light chain | CR8043 | Friesen, R. H. et al., A common solution | 24145 |
| | | | to group 2 influenza virus neutralization; | |
| | | | Proc. Natl. Acad. Sci. U.S.A. 111 (1), | |
| | | | 445-450 (2014), NCBI Accession | |
| | | | #4NM8_L | |
| INFL645 | Light chain | CR8059 | Dreyfus, C. et al., Highly conserved | 24146 |
| | | | protective epitopes on influenza B | |
| | | | viruses; Science 337 (6100), 1343-1348 | |
| | | | (2012), NCBI Accession #4FQK_L | |
| INFL646 | Light chain | CR8071 | Dreyfus, C. et al., Highly conserved | 24147 |
| | | | protective epitopes on influenza B | |
| | | | viruses; Science 337 (6100), 1343-1348 | |
| | | | (2012), NCBI Accession # 4FQJ_L | 24148 |
| | | | (216aa) | |
| INFL647 | Light chain | CR9114 | WO2013079473; WO2014191435; | 24149 |
| | | | Dreyfus, C., Laursen, N. S. et al., Highly | |
| | | | conserved protective epitopes on | |
| | | | influenza B viruses; Science 337 (6100), | |
| | | | 1343-1348 (2012), NCBI Accession | |
| | | | #4FQY_L(216aa) | |
| INFL648 | Light chain | Ch67 | Schmidt, A. G., et al., Preconfiguration of | 24150 |
| | | | the antigen-binding site during affinity | |
| | | | maturation of a broadly neutralizing | |
| | | | influenza virus antibody; Proc. Natl. | |
| | | | Acad. Sci. U.S.A. 110 (1), 264-269 | |
| | | | (2013), NCBI Accession #4HKX_B | |
| | | | (214aa) | |
| INFL649 | Light chain | Fab 26/9 | Schulze-Gahmen, U. et al., J. Biol. | 24151 |
| | | | Chem. 263 (32), 17100-17105 (1988); | |
| | | | Churchill, M. E., et al., J. Mol. Biol. 241 | |
| | | | (4), 534-556 (1994), NCBI Accession | |
| | | | #LFRG_L | |
| INFL650 | Light chain | Fab 3.1 | Wyrzucki, A. et al., Alternative | 24152 |
| | | | Recognition of the Conserved Stem | |
| | | | Epitope in Influenza A Virus | |
| | | | Hemagglutinin by a VH3-30-Encoded | |
| | | | Heterosubtypic Antibody; J. Virol. 88 | |
| | | | (12), 7083-7092 (2014), NCBI Accession | |
| | | | #4PY8_J | |
| INFL651 | Light chain | Fab 2g1 | Xu, R. et al., A recurring motif for | 24153 |
| | | | antibody recognition of the receptor- | |
| | | | binding site of influenza hemagglutinin; | |
| | | | Nat. Struct. Mol. Biol. 20 (3), 363-370 | |
| | | | (2013), NCBI Accession #4HG4_M | |
| | | | (214aa) | |
| INFL652 | Light chain | Fab 8m2 | Xu, R. et al., A recurring motif for | 24154 |
| | | | antibody recognition of the receptor- | |
| | | | binding site of influenza hemagglutinin; | |
| | | | Nat. Struct. Mol. Biol. 20 (3), 363-370 | |
| | | | (2013), NCBI Accession #4HFU_L | |
| | | | (215aa) | |
| INFL653 | Light chain | Fab 8f8 | Xu, R. et al., A recurring motif for | 24155 |
| | | | antibody recognition of the receptor- | |
| | | | binding site of influenza hemagglutinin; | |
| | | | Nat. Struct. Mol. Biol. 20 (3), 363-370 | |
| | | | (2013), NCBI Accession # 4HF5_L | |
| | | | (218aa) | |
| INFL654 | Light chain | Fab 2d1 | Xu, R., et al., Structural basis of | 24156 |
| | | | preexisting immunity to the 2009 H1N1 | |
| | | | pandemic influenza virus; Science 328 | |
| | | | (5976), 357-360 (2010), NCBI Accession | |
| | | | #3LZF_L (217aa) | |
| INFL655 | Light chain | Fi6v3 | Corti, D. et al., A neutralizing antibody | 24157 |
| | | | selected from plasma cells that binds to | |
| | | | group 1 and group 2 influenza A | |
| | | | hemagglutinins; Science 333 (6044), | |
| | | | 850-856 (2011), NCBI Accession | |
| | | | #3ZTN_L | |
| INFL656 | Light chain | Light chain from | Iba, Y., et al., Conserved Neutralizing | 24158 |
| | | 3WHE_N | Epitope at Globular Head of | |
| | | | Hemagglutinin in H3N2 Influenza | |
| | | | Viruses; J. Virol. (2014), NCBI | 24159 |
| | | | Accession #3WHE_N (220aa) | |
| INFL657 | Light chain | monoclonal | Burioni, R. et al., Monoclonal antibodies | 24160 |
| | (partial) | antibody PN- | isolated from human B cells neutralize a | |
| | | SIA28 | broad range of H1 subtype influenza A | |
| | | | viruses including swine-origin Influenza | |
| | | | virus(S-OIV); Virology (2010), NCBI | |
| | | | Accession #ACX30939.1 (105aa) | |
| INFL658 | Light chain | monoclonal | Burioni, R. et al., Monoclonal antibodies | 24161 |
| | (partial) | antibody PN- | isolated from human B cells neutralize a | |
| | | SIA49 | broad range of H1 subtype influenza A | |
| | | | viruses including swine-origin Influenza | |
| | | | virus(S-OIV); Virology (2010), NCBI | |
| | | | Accession #ACX30938.1 (105aa) | |
| INFL659 | Light chain; Fab | 5j8 | Hong, M. et al., Antibody Recognition of | 24162 |
| | | | the Pandemic H1N1 Influenza Virus | |
| | | | Hemagglutinin Receptor Binding Site; J. | |
| | | | Virol. 87 (22), 12471-12480 (2013), | |
| | | | NCBI Accession #4M5Z_L | |
| INFL660 | Light chain CDR 1 | Ab1A2 | WO2015028478 SEQ ID 9 | 24163 |
| INFL661 | Light chain CDR 2 | Ab1A2 | WO2015028478 SEQ ID 10 | 24164 |
| INFL662 | Light chain CDR 3 | Ab1A2 | WO2015028478 SEQ ID 11 | 24165 |
| INFL663 | Light chain Fab | CT147 | WO2013048153, US20140234336 SEQ | 24166 |
| | | | ID NO: 37 | |
| INFL664 | Light chain Fab | CT164 | WO2013048153, US20140234336 SEQ | 24167 |
| | | | ID NO: 41 | |
| INFL665 | Light chain Fab | CT166 | WO2013048153, US20140234336 SEQ | 24168 |
| | | | ID NO: 43 | |
| INFL666 | Light chain Human | Fab 39.29 | Nakamura, G. et al., An in vivo human- | 24169 |
| | igg | | plasmablast enrichment technique allows | |
| | | | rapid identification of therapeutic | |
| | | | influenza a antibodies; Cell Host | |
| | | | Microbe 14 (1), 93-103 (2013), NCBI | |
| | | | Accession #4KVN_L (215aa) | |
| INFL667 | Light chain K3 | h2B11 | U.S. Pat. No. 9,115,201 SEQ ID NO: 9 | 24170 |
| INFL668 | Light chain K3 | h2B12 | U.S. Pat. No. 9,115,201 SEQ ID NO: 10 | 24171 |
| INFL669 | Light chain partial | 4A10 | Krause, J. C. et al. “Epitope-specific | 24172 |
| | region | | human influenza antibody repertoires | |
| | | | diversify by B cell intraclonal sequence | |
| | | | divergence and interclonal convergence” | |
| | | | J. Immunol. 187 (7), 3704-3711 (2011), | |
| | | | NCBI Accession #AEO16798 | |
| INFL670 | Light chain variable | LC-VD from | US2013030234 SEQ ID NO: 33 | 24173 |
| | (exemplary) | US2013030234 | | |
| INFL671 | Light chain variable | LC-VD from | US2013030234 SEQ ID NO: 34 | 24174 |
| | (exemplary) | US2013030234 | | |
| INFL672 | Light chain variable | LC-VD from | US2013030234 SEQ ID NO: 35 | 24175 |
| | (exemplary) | US2013030234 | | |
| INFL673 | Light chain variable | LC-VD from | US2013030234 SEQ ID NO: 36 | 24176 |
| | (exemplary) | US2013030234 | | |
| INFL674 | Light chain variable | LC-VD from | US2013030234 SEQ ID NO: 37 | 24177 |
| | (exemplary) | US2013030234 | | |
| INFL675 | Light chain variable | LC-VD from | US2013030234 SEQ ID NO: 38 | 24178 |
| | (exemplary) | US2013030234 | | |
| INFL676 | Light chain variable | LC-VD from | US2013030234 SEQ ID NO: 39 | 24179 |
| | (exemplary) | US2013030234 | | |
| INFL677 | Light chain variable | LC-VD from | US2013030234 SEQ ID NO: 40 | 24180 |
| | (exemplary) | US2013030234 | | |
| INFL678 | Light chain variable | LC-VD from | US2013030234 SEQ ID NO: 41 | 24181 |
| | (exemplary) | US2013030234 | | |
| INFL679 | Light chain variable | LC-VD from | US2013030234 SEQ ID NO: 42 | 24182 |
| | (exemplary) | US2013030234 | | |
| INFL680 | Light chain variable | LC-VD from | US2013030234 SEQ ID NO: 43 | 24183 |
| | (exemplary) | US2013030234 | | |
| INFL681 | Light chain variable | 39.18 B11 | US20140161822 SEQ ID NO: 156 | 24184 |
| | region | | | |
| INFL682 | Light chain variable | GG3 | WO2014159960 SEQ ID NO: 25 | 24185 |
| | region | | | |
| INFL683 | Light chain variable | N547 | U.S. Pat. No. 8,003,106 SEQ ID NO: 29 | 24186 |
| | region | | | |
| INFL684 | Light chain variable | L66 | U.S. Pat. No. 8,003,106 SEQ ID NO: 31 | 24187 |
| | region | | | |
| INFL685 | Light chain variable | C40 | U.S. Pat. No. 8,003,106 SEQ ID NO: 27 | 24188 |
| | region | | | |
| INFL686 | Light chain variable | 14C2 | U.S. Pat. No. 8,080,244 SEQ ID NO: 7 | 24189 |
| | region | | | |
| INFL687 | Light chain variable | h14C2 | U.S. Pat. No. 8,080,244 SEQ ID NO: 1 | 24190 |
| | region | | | |
| INFL688 | Light chain variable | VN04-2-HuG1 | US20100150941 SEQ ID NO: 6 | 24191 |
| | region | | | |
| INFL689 | Light chain variable | VN04-3-HuG1 | US20100150941 SEQ ID NO: 8 | 24192 |
| | region | | | |
| INFL690 | Light chain variable | FI6 variant 1, FI6 | U.S. Pat. No. 8,871,207 SEQ ID NO: 14 | 24193 |
| | region | variant 2 | | |
| INFL691 | Light chain variable | FI6 variant 3, FI6 | U.S. Pat. No. 8,871,207 SEQ ID NO: 57 | 24194 |
| | region | variant 4 | | |
| INFL692 | Light chain variable | FI6 variant 5 | U.S. Pat. No. 8,871,207 SEQ ID NO: 61 | 24195 |
| | region | | | |
| INFL693 | Light chain variable | FI28 vanant 1, | U.S. Pat. No. 8,871,207 SEQ ID NO: 30 | 24196 |
| | region | FI28 variant 2 | | |
| INFL694 | Light chain variable | 21B15 | U.S. Pat. No. 8,858,948 SEQ ID NO: 46 | 24197 |
| | region | | | |
| INFL695 | Light chain variable | 3241_G23 | U.S. Pat. No. 8,858,948 SEQ ID NO: 118 | 24198 |
| | region | | | |
| INFL696 | Light chain variable | 3244_I10 | U.S. Pat. No. 8,858,948 SEQ ID NO: 122 | 24199 |
| | region | | | |
| INFL697 | Light chain variable | 3243_J07 | U.S. Pat. No. 8,858,948 SEQ ID NO: 126 | 24200 |
| | region | | | |
| INFL698 | Light chain variable | 3259_J21 | U.S. Pat. No. 8,858,948 SEQ ID NO: 130 | 24201 |
| | region | | | |
| INFL699 | Light chain variable | 3245_O19 | U.S. Pat. No. 8,858,948 SEQ ID NO: 134 | 24202 |
| | region | | | |
| INFL700 | Light chain variable | 3244_H04 | U.S. Pat. No. 8,858,948 SEQ ID NO: 138 | 24203 |
| | region | | | |
| INFL701 | Light chain variable | 3136_G05 | U.S. Pat. No. 8,858,948 SEQ ID NO: 142 | 24204 |
| | region | | | |
| INFL702 | Light chain variable | 3252_C13 | U.S. Pat. No. 8,858,948 SEQ ID NO: 146 | 24205 |
| | region | | | |
| INFL703 | Light chain variable | 3255_J06 | U.S. Pat. No. 8,858,948 SEQ ID NO: 150 | 24206 |
| | region | | | |
| INFL704 | Light chain variable | 3420_I23 | U.S. Pat. No. 8,858,948 SEQ ID NO: 154 | 24207 |
| | region | | | |
| INFL705 | Light chain variable | 3248_P18 | U.S. Pat. No. 8,858,948 SEQ ID NO: 160 | 24208 |
| | region | | | |
| INFL706 | Light chain variable | 3253_P10 | U.S. Pat. No. 8,858,948 SEQ ID NO: 164 | 24209 |
| | region | | | |
| INFL707 | Light chain variable | 3260_D19 | U.S. Pat. No. 8,858,948 SEQ ID NO: 168 | 24210 |
| | region | | | |
| INFL708 | Light chain variable | 3362_B11 | U.S. Pat. No. 8,858,948 SEQ ID NO: 174 | 24211 |
| | region | | | |
| INFL709 | Light chain variable | 3242_P05 | U.S. Pat. No. 8,858,948 SEQ ID NO: 178 | 24212 |
| | region | | | |
| INFL710 | Light chain variable | A66 | WO2009079259, US20110038935, | 24213 |
| | region | | US20140011982 SEQ ID NO: 34 | |
| INFL711 | Light chain variable | D7, H98 | WO2009079259, US20110038935, | 24214 |
| | region | | US20140011982 SEQ ID NO: 8 | |
| INFL712 | Light chain variable | D8 | WO2009079259, US20110038935, | 24215 |
| | region | | US20140011982 SEQ ID NO: 14 | |
| INFL713 | Light chain variable | D80 | WO2009079259, US20110038935, | 24216 |
| | region | | US20140011982 SEQ ID NO: 16 | |
| INFL714 | Light chain variable | E88 | WO2009079259, US20110038935, | 24217 |
| | region | | US20140011982 SEQ ID NO: 38 | |
| INFL715 | Light chain variable | E90 | WO2009079259, US20110038935, | 24218 |
| | region | | US20140011982 SEQ ID NO: 22 | |
| INFL716 | Light chain variable | F10 | WO2009079259, US20110038935, | 24219 |
| | region | | US20140011982 SEQ ID NO: 20 | |
| INFL717 | Light chain variable | G17 | WO2009079259, US20110038935, | 24220 |
| | region | | US20140011982 SEQ ID NO: 26 | |
| INFL718 | Light chain variable | H40 | WO2009079259, US20110038935, | 24221 |
| | region | | US20140011982 SEQ ID NO: 30 | |
| INFL719 | Light chain variable | H98 | WO2009079259, US20110038935, | 24222 |
| | region | | US20140011982 SEQ ID NO: 10 | |
| INFL720 | Light chain variable | CH65 | WO2013020074, US20140302043 SEQ | 24223 |
| | region | | ID NO: 10 | |
| INFL721 | Light chain variable | CH66 | WO2013020074, US20140302043 SEQ | 24224 |
| | region | | ID NO: 11 | |
| INFL722 | Light chain variable | CH67 | WO2013020074, US20140302043 SEQ | 24225 |
| | region | | ID NO: 12 | |
| INFL723 | Light chain variable | CL86OUCA | WO2013020074, US20140302043 SEQ | 24226 |
| | region | | ID NO: 9 | |
| INFL724 | Light chain variable | Antibody 1 | WO2015051010 SEQ ID NO: 7 | 24227 |
| | region | | | |
| INFL725 | Light chain variable | Antibody 2 | WO2015051010 SEQ ID NO: 17 | 24228 |
| | region | | | |
| INFL726 | Light chain variable | Antibody 3 | WO2015051010 SEQ ID NO: 27 | 24229 |
| | region | | | |
| INFL727 | Light chain variable | Antibody 4 | WO2015051010 SEQ ID NO: 37 | 24230 |
| | region | | | |
| INFL728 | Light chain variable | Antibody 5 | WO2015051010 SEQ ID NO: 47 | 24231 |
| | region | | | |
| INFL729 | Light chain variable | Antibody 6 | WO2015051010 SEQ ID NO: 57 | 24232 |
| | region | | | |
| INFL730 | Light chain variable | Antibody 7 | WO2015051010 SEQ ID NO: 67 | 24233 |
| | region | | | |
| INFL731 | Light chain variable | Antibody 8 | WO2015051010 SEQ ID NO: 77 | 24234 |
| | region | | | |
| INFL732 | Light chain variable | Antibody 9 | WO2015051010 SEQ ID NO: 87 | 24235 |
| | region | | | |
| INFL733 | Light chain variable | Antibody 10 | WO2015051010 SEQ ID NO: 97 | 24236 |
| | region | | | |
| INFL734 | Light chain variable | Antibody 11 | WO2015051010 SEQ ID NO: 107 | 24237 |
| | region | | | |
| INFL735 | Light chain variable | Antibody 12 | WO2015051010 SEQ ID NO: 117 | 24238 |
| | region | | | |
| INFL736 | Light chain variable | Antibody 13 | WO2015051010 SEQ ID NO: 127 | 24239 |
| | region | | | |
| INFL737 | Light chain variable | Antibody 14 | WO2015051010 SEQ ID NO: 137 | 24240 |
| | region | | | |
| INFL738 | Light chain variable | Antibody 15 | WO2015051010 SEQ ID NO: 147 | 24241 |
| | region | | | |
| INFL739 | Light chain variable | Antibody 3-GL | WO2015051010 SEQ ID NO: 157 | 24242 |
| | region | | | |
| INFL740 | Light chain variable | 005-2G02 | WO2013059524, US20140348851 SEQ | 24243 |
| | region | | ID NO: 11 | |
| INFL741 | Light chain variable | 005-2G02 | WO2013059524, US20140348851 SEQ | 24244 |
| | region | | ID NO: 19 | |
| INFL742 | Light chain variable | 09-2A06 | WO2013059524, US20140348851 SEQ | 24245 |
| | region | | ID NO: 31 | |
| INFL743 | Light chain variable | 09-2A06 | WO2013059524, US20140348851 SEQ | 24246 |
| | region | | ID NO: 39 | |
| INFL744 | Light chain variable | 09-3A01 | WO2013059524, US20140348851 SEQ | 24247 |
| | region | | ID NO: 51 | |
| INFL745 | Light chain variable | 09-3A01 | WO2013059524, US20140348851 SEQ | 24248 |
| | region | | ID NO: 59 | |
| INFL746 | Light chain variable | 70-IF02 | WO2012096994, US20140046039 SEQ | 24249 |
| | region | | ID NO: 21 | |
| INFL747 | Light chain variable | | US20120058124 SEQ ID NO: 15 | 24250 |
| | region | | | |
| INFL748 | Light chain variable | | US20120058124 SEQ ID NO: 16 | 24251 |
| | region | | | |
| INFL749 | Light chain variable | | US20120058124 SEQ ID NO: 17 | 24252 |
| | region | | | |
| INFL750 | Light chain variable | | US20120058124 SEQ ID NO: 18 | 24253 |
| | region | | | |
| INFL751 | Light chain variable | | US20120058124 SEQ ID NO: 19 | 24254 |
| | region | | | |
| INFL752 | Light chain variable | | US20120058124 SEQ ID NO: 20 | 24255 |
| | region | | | |
| INFL753 | Light chain variable | | US20120058124 SEQ ID NO: 21 | 24256 |
| | region | | | |
| INFL754 | Light chain variable | | US20120058124 SEQ ID NO: 22 | 24257 |
| | region | | | |
| INFL755 | Light chain variable | | US20120058124 SEQ ID NO: 23 | 24258 |
| | region | | | |
| INFL756 | Light chain variable | | US20120058124 SEQ ID NO: 24 | 24259 |
| | region | | | |
| INFL757 | Light chain variable | | US20120058124 SEQ ID NO: 25 | 24260 |
| | region | | | |
| INFL758 | Light chain variable | | US20120058124 SEQ ID NO: 26 | 24261 |
| | region | | | |
| INFL759 | Light chain variable | | US20120058124 SEQ ID NO: 70 | 24262 |
| | region | | | |
| INFL760 | Light chain variable | 81.39 | US20140161822, US20140248286, | 24263 |
| | region | | WO2014078268 SEQ ID NO: 113 | |
| INFL761 | Light chain variable | 81.39 | US20140161822, US20140248286, | 24264 |
| | region | | WO2014078268 SEQ ID NO: 117 | |
| INFL762 | Light chain variable | 81.39 | US20140161822, US20140248286, | 24265 |
| | region | | WO2014078268 SEQ ID NO: 119 | |
| INFL763 | Light chain variable | 81.39 | US20140161822, US20140248286, | 24266 |
| | region | | WO2014078268 SEQ ID NO: 122 | |
| INFL764 | Light chain variable | 81.39 | US20140161822, US20140248286, | 24267 |
| | region | | WO2014078268 SEQ ID NO: 124 | |
| INFL765 | Light chain variable | 81.39 | US20140161822, US20140248286, | 24268 |
| | region | | WO2014078268 SEQ ID NO: 126 | |
| INFL766 | Light chain variable | 81.39 | US20140161822, US20140248286, | 24269 |
| | region | | WO2014078268 SEQ ID NO: 128 | |
| INFL767 | Light chain variable | 81.39 | US20140161822, US20140248286, | 24270 |
| | region | | WO2014078268 SEQ ID NO: 130 | |
| INFL768 | Light chain variable | 81.39 | US20140161822, US20140248286, | 24271 |
| | region | | WO2014078268 SEQ ID NO: 132 | |
| INFL769 | Light chain variable | 39.29 | US20140161822, US20140248286, | 24272 |
| | region | | WO2014078268 SEQ ID NO: 136 | |
| INFL770 | Light chain variable | 39.29 | US20140161822, US20140248286, | 24273 |
| | region | | WO2014078268 SEQ ID NO: 140 | |
| INFL771 | Light chain variable | 39.29 | US20140161822, US20140248286, | 24274 |
| | region | | WO2014078268 SEQ ID NO: 144 | |
| INFL772 | Light chain variable | 39.29 | US20140161822, US20140248286, | 24275 |
| | region | | WO2014078268 SEQ ID NO: 146 | |
| INFL773 | Light chain variable | 39.29 | US20140161822, US20140248286, | 24276 |
| | region | | WO2014078268 SEQ ID NO: 150 | |
| INFL774 | Light chain variable | 39.29 | US20140161822, US20140248286, | 24277 |
| | region | | WO2014078268 SEQ ID NO: 152 | |
| INFL775 | Light chain variable | 36.89 | US20140161822, US20140248286, | 24278 |
| | region | | WO2014078268 SEQ ID NO: 162 | |
| INFL776 | Light chain variable | 9.01F3 | US20140161822, US20140248286, | 24279 |
| | region | | WO2014078268 SEQ ID NO: 166 | |
| INFL777 | Light chain variable | 23.06C2 | US20140161822, US20140248286, | 24280 |
| | region | | WO2014078268 SEQ ID NO: 170 | |
| INFL778 | Light chain variable | 39.29 | US20140161822, US20140248286, | 24281 |
| | region | | WO2014078268 SEQ ID NO: 235 | |
| INFL779 | Light chain variable | F16 Variant 3 | WO2013011347, US20140271655, | 24282 |
| | region | | U.S. Pat. No. 8,871,207 SEQ ID NO: 57 | |
| INFL780 | Light chain variable | F16 Variant 5 | WO2013011347, US20140271655, | 24283 |
| | region | | U.S. Pat. No. 8,871,207 SEQ ID NO: 61 | |
| INFL781 | Light chain variable | FC41 | WO2010010467 SEQ ID NO 61 | 24284 |
| | region | | | |
| INFL782 | Light chain variable | FE43 | WO2010010467 SEQ ID NO 75 | 24285 |
| | region | | | |
| INFL783 | Light chain variable | FB75, FB110, | WO2010010467 SEQ ID NO 122 | 24286 |
| | region | FB177 | | |
| INFL784 | Light chain variable | FB17 | WO2010010467 SEQ ID NO 106 | 24287 |
| | region | | | |
| INFL785 | Light chain variable | FC6 | WO2010010467 SEQ ID NO 46 | 24288 |
| | region | | | |
| INFL786 | Light chain variable | FE53 | WO2010010467 SEQ ID NO 90 | 24289 |
| | region | | | |
| INFL787 | Light chain variable | 7A7 | WO2010138564 SEQ ID NO: 7 | 24290 |
| | region | | | |
| INFL788 | Light chain variable | 12DI | WO2010138564 SEQ ID NO: 13 | 24291 |
| | region | | | |
| INFL789 | Light chain variable | 66A6 | WO2010138564 SEQ ID NO: 17 | 24292 |
| | region | | | |
| INFL790 | Light chain variable | B-1 | U.S. Pat. No. 8,975,378, US20110319600, | 24293 |
| | region | | WO2010073647 SEQ ID NO: 28 | |
| INFL791 | Light chain variable | D1 | U.S. Pat. No. 8,975,378, US20110319600, | 24294 |
| | region | | WO2010073647 SEQ ID NO: 30 | |
| INFL792 | Light chain variable | E-2 | U.S. Pat. No. 8,975,378, US20110319600, | 24295 |
| | region | | WO2010073647 SEQ ID NO: 32 | |
| INFL793 | Light chain variable | B-3 | U.S. Pat. No. 8,975,378, US20110319600, | 24296 |
| | region | | WO2010073647 SEQ ID NO: 34 | |
| INFL794 | Light chain variable | 5A7 | WO2013114885, US20140377262 SEQ | 24297 |
| | region | | ID NO: 35 | |
| INFL795 | Light chain variable | 3A2 | WO2013114885, US20140377262 SEQ | 24298 |
| | region | | ID NO: 39 | |
| INFL796 | Light chain variable | 10C4 | WO2013114885, US20140377262 SEQ | 24299 |
| | region | | ID NO: 43 | |
| INFL797 | Light chain variable | Fab49 | WO2009144667, US20110076265 SEQ | 24300 |
| | region | | ID NO: 2 | |
| INFL798 | Light chain variable | Fab28, IgG PN- | WO2009115972, WO2011117848, | 24301 |
| | region | SIA28 | US20110014187 SEQ ID NO: 2 | |
| INFL799 | Light chain variable | TCN-522 | US20120207760, U.S. Pat. No. 8,916,160 SEQ ID | 24302 |
| | region | | NO: 778; U.S. Pat. No. 8,900,590 SEQ ID NO: 33 | |
| INFL800 | Light chain variable | CR8018 | WO2010130636 SEQ ID NO: 24 | 24303 |
| | region | | | |
| INFL801 | Light chain variable | CR8019 | WO2010130636 SEQ ID NO: 28 | 24304 |
| | region | | | |
| INFL802 | Light chain variable | CR8020 | WO2010130636 SEQ ID NO: 32 | 24305 |
| | region | | | |
| INFL803 | Light chain variable | CR8021 | WO2010130636 SEQ ID NO: 36 | 24306 |
| | region | | | |
| INFL804 | Light chain variable | CR8038 | WO2010130636 SEQ ID NO: 40 | 24307 |
| | region | | | |
| INFL805 | Light chain variable | CR8039 | WO2010130636 SEQ ID NO: 44 | 24308 |
| | region | | | |
| INFL806 | Light chain variable | CR8040 | WO2010130636 SEQ ID NO: 48 | 24309 |
| | region | | | |
| INFL807 | Light chain variable | CR8041 | WO2010130636 SEQ ID NO: 52 | 24310 |
| | region | | | |
| INFL808 | Light chain variable | CR8043 | WO2010130636 SEQ ID NO: 56 | 24311 |
| | region | | | |
| INFL809 | Light chain variable | CR8049 | WO2010130636 SEQ ID NO: 59 | 24312 |
| | region | | | |
| INFL810 | Light chain variable | CR8050 | WO2010130636 SEQ ID NO: 63 | 24313 |
| | region | | | |
| INFL811 | Light chain variable | CR8052 | WO2010130636 SEQ ID NO: 67 | 24314 |
| | region | | | |
| INFL812 | Light chain variable | CR8055 | WO2010130636 SEQ ID NO: 71 | 24315 |
| | region | | | |
| INFL813 | Light chain variable | CR8057 | WO2010130636 SEQ ID NO: 75 | 24316 |
| | region | | | |
| INFL814 | Light chain variable | CR8069 | WO2010130636 SEQ ID NO: 79 | 24317 |
| | region | | | |
| INFL815 | Light chain variable | CR6255 | US20090311265, U.S. Pat. No. 8,691,223, | 24318 |
| | region | | U.S. Pat. No. 9,109,017, WO2008028946 SEQ ID | |
| | | | NO: 85 | |
| INFL816 | Light chain variable | CR6257 | US20090311265, U.S. Pat. No. 8,691,223, | 24319 |
| | region | | U.S. Pat. No. 9,109,017, WO2008028946 SEQ ID | |
| | | | NO: 87 | |
| INFL817 | Light chain variable | CR6260 | US20090311265, U.S. Pat. No. 8,691,223, | 24320 |
| | region | | U.S. Pat. No. 9,109,017, WO2008028946 SEQ ID | |
| | | | NO: 89 | |
| INFL818 | Light chain variable | CR6261 | US20090311265, U.S. Pat. No. 8,691,223, | 24321 |
| | region | | U.S. Pat. No. 9,109,017, WO2008028946 SEQ ID | |
| | | | NO: 91 | |
| INFL819 | Light chain variable | CR6262 | US20090311265, U.S. Pat. No. 8,691,223, | 24322 |
| | region | | U.S. Pat. No. 9,109,017, WO2008028946 SEQ ID | |
| | | | NO: 93 | |
| INFL820 | Light chain variable | CR6268 | US20090311265, U.S. Pat. No. 8,691,223, | 24323 |
| | region | | U.S. Pat. No. 9,109,017, WO2008028946 SEQ ID | |
| | | | NO: 95 | |
| INFL821 | Light chain variable | CR6307 | US20090311265, U.S. Pat. No. 8,691,223, | 24324 |
| | region | | U.S. Pat. No. 9,109,017, WO2008028946 SEQ ID | |
| | | | NO: 97 | |
| INFL822 | Light chain variable | CR6310 | US20090311265, U.S. Pat. No. 8,691,223, | 24325 |
| | region | | U.S. Pat. No. 9,109,017, WO2008028946 SEQ ID | |
| | | | NO: 99 | |
| INFL823 | Light chain variable | CR6314 | US20090311265, U.S. Pat. No. 8,691,223, | 24326 |
| | region | | U.S. Pat. No. 9,109,017, WO2008028946 SEQ ID | |
| | | | NO: 101 | |
| INFL824 | Light chain variable | CR6323 | US20090311265, U.S. Pat. No. 8,691,223, | 24327 |
| | region | | U.S. Pat. No. 9,109,017, WO2008028946 SEQ ID | |
| | | | NO: 103 | |
| INFL825 | Light chain variable | CR6325 | US20090311265, U.S. Pat. No. 8,691,223, | 24328 |
| | region | | U.S. Pat. No. 9,109,017, WO2008028946 SEQ ID | |
| | | | NO: 105 | |
| INFL826 | Light chain variable | CR6331 | US20090311265, U.S. Pat. No. 8,691,223, | 24329 |
| | region | | U.S. Pat. No. 9,109,017, WO2008028946 SEQ ID | |
| | | | NO: 107 | |
| INFL827 | Light chain variable | CR6344 | US20090311265, U.S. Pat. No. 8,691,223, | 24330 |
| | region | | U.S. Pat. No. 9,109,017, WO2008028946 SEQ ID | |
| | | | NO: 109 | |
| INFL828 | Light chain variable | CR6141 | US20090311265, U.S. Pat. No. 8,691,223, | 24331 |
| | region | | U.S. Pat. No. 9,109,017, WO2008028946 SEQ ID | |
| | | | NO: 319 | |
| INFL829 | Light chain variable | CR6272 | US20090311265, U.S. Pat. No. 8,691,223, | 24332 |
| | region | | U.S. Pat. No. 9,109,017, WO2008028946 SEQ ID | |
| | | | NO: 323 | |
| INFL830 | Light chain variable | CR6296 | US20090311265, U.S. Pat. No. 8,691,223, | 24333 |
| | region | | U.S. Pat. No. 9,109,017, WO2008028946 SEQ ID | |
| | | | NO: 327 | |
| INFL831 | Light chain variable | CR6301 | US20090311265, U.S. Pat. No. 8,691,223, | 24334 |
| | region | | U.S. Pat. No. 9,109,017, WO2008028946 SEQ ID | |
| | | | NO: 331 | |
| INFL832 | Light chain variable | CR6327 | US20090311265, U.S. Pat. No. 8,691,223, | 24335 |
| | region | | U.S. Pat. No. 9,109,017, WO2008028946 SEQ ID | |
| | | | NO: 335 | |
| INFL833 | Light chain variable | CR6328 | US20090311265, U.S. Pat. No. 8,691,223, | 24336 |
| | region | | U.S. Pat. No. 9,109,017, WO2008028946 SEQ ID | |
| | | | NO: 339 | |
| INFL834 | Light chain variable | CR6329 | US20090311265, U.S. Pat. No. 8,691,223, | 24337 |
| | region | | U.S. Pat. No. 9,109,017, WO2008028946 SEQ ID | |
| | | | NO: 343 | |
| INFL835 | Light chain variable | CR6332 | US20090311265, U.S. Pat. No. 8,691,223, | 24338 |
| | region | | U.S. Pat. No. 9,109,017, WO2008028946 SEQ ID | |
| | | | NO: 347 | |
| INFL836 | Light chain variable | CR6334 | US20090311265, U.S. Pat. No. 8,691,223, | 24339 |
| | region | | U.S. Pat. No. 9,109,017, WO2008028946 SEQ ID | |
| | | | NO: 351 | |
| INFL837 | Light chain variable | CR6336 | US20090311265, U.S. Pat. No. 8,691,223, | 24340 |
| | region | | U.S. Pat. No. 9,109,017, WO2008028946 SEQ ID | |
| | | | NO: 355 | |
| INFL838 | Light chain variable | CR6339 | US20090311265, U.S. Pat. No. 8,691,223, | 24341 |
| | region | | U.S. Pat. No. 9,109,017, WO2008028946 SEQ ID | |
| | | | NO: 359 | |
| INFL839 | Light chain variable | CR6342 | US20090311265, U.S. Pat. No. 8,691,223, | 24342 |
| | region | | U.S. Pat. No. 9,109,017, WO2008028946 SEQ ID | |
| | | | NO: 363 | |
| INFL840 | Light chain variable | CR6343 | US20090311265, U.S. Pat. No. 8,691,223, | 24343 |
| | region | | U.S. Pat. No. 9,109,017, WO2008028946 SEQ ID | |
| | | | NO: 367 | |
| INFL841 | Light chain variable | CR9003 | US20140120113 SEQ ID NO: 4 | 24344 |
| | region | | | |
| INFL842 | Light chain variable | CR9004 | US20140120113 SEQ ID NO: 8 | 24345 |
| | region | | | |
| INFL843 | Light chain variable | CR9005 | US20140120113 SEQ ID NO: 12 | 24346 |
| | region | | | |
| INFL844 | Light chain variable | CR9006 | US20140120113 SEQ ID NO: 16 | 24347 |
| | region | | | |
| INFL845 | Light chain variable | CR9007 | US20140120113 SEQ ID NO: 20 | 24348 |
| | region | | | |
| INFL846 | Light chain variable | CR9008 | US20140120113 SEQ ID NO: 24 | 24349 |
| | region | | | |
| INFL847 | Light chain variable | CR9009 | US20140120113 SEQ ID NO: 28 | 24350 |
| | region | | | |
| INFL848 | Light chain variable | CR9010 | US20140120113 SEQ ID NO: 32 | 24351 |
| | region | | | |
| INFL849 | Light chain variable | CR9011 | US20140120113 SEQ ID NO: 36 | 24352 |
| | region | | | |
| INFL850 | Light chain variable | CR9012 | US20140120113 SEQ ID NO: 40 | 24353 |
| | region | | | |
| INFL851 | Light chain variable | CR9029 | US20140120113 SEQ ID NO: 44 | 24354 |
| | region | | | |
| INFL852 | Light chain variable | CR9030 | US20140120113 SEQ ID NO: 48 | 24355 |
| | region | | | |
| INFL853 | Light chain variable | CR9031 | US20140120113 SEQ ID NO: 52 | 24356 |
| | region | | | |
| INFL854 | Light chain variable | CR9112 | US20140120113 SEQ ID NO: 56 | 24357 |
| | region | | | |
| INFL855 | Light chain variable | CR9113 | US20140120113 SEQ ID NO: 60 | 24358 |
| | region | | | |
| INFL856 | Light chain variable | CR9114 | US20140120113 SEQ ID NO: 64 | 24359 |
| | region | | | |
| INFL857 | Light chain variable | CR8033 | U.S. Pat. No. 8,852,595 SEQ ID NO: 73 | 24360 |
| | region | | | |
| INFL858 | Light chain variable | CR8059 | U.S. Pat. No. 8,852,595 SEQ ID NO: 77 | 24361 |
| | region | | | |
| INFL859 | Light chain variable | CR8071 | U.S. Pat. No. 8,852,595 SEQ ID NO: 79 | 24362 |
| | region | | | |
| INFL860 | Light chain variable | CR10051 | U.S. Pat. No. 8,852,595 SEQ ID NO: 83 | 24363 |
| | region | | | |
| INFL861 | Light chain variable | CR10049 | U.S. Pat. No. 8,852,595 SEQ ID NO: 87 | 24364 |
| | region | | | |
| INFL862 | Light chain variable | CR10023 | U.S. Pat. No. 8,852,595 SEQ ID NO: 91 | 24365 |
| | region | | | |
| INFL863 | Light chain variable | CR10032 | U.S. Pat. No. 8,852,595 SEQ ID NO: 95 | 24366 |
| | region | | | |
| INFL864 | Light chain variable | CR11035 | U.S. Pat. No. 8,852,595 SEQ ID NO: 103 | 24367 |
| | region | | | |
| INFL865 | Light chain variable | CR11036 | U.S. Pat. No. 8,852,595 SEQ ID NO: 107 | 24368 |
| | region | | | |
| INFL866 | Light chain variable | CR11038 | U.S. Pat. No. 8,852,595 SEQ ID NO: 111 | 24369 |
| | region | | | |
| INFL867 | Light chain variable | CR11039 | U.S. Pat. No. 8,852,595 SEQ ID NO: 115 | 24370 |
| | region | | | |
| INFL868 | Light chain variable | CR8031 | U.S. Pat. No. 8,852,595 SEQ ID NO: 121 | 24371 |
| | region | | | |
| INFL869 | Light chain variable | CR8032 | U.S. Pat. No. 8,852,595 SEQ ID NO: 125 | 24372 |
| | region | | | |
| INFL870 | Light chain variable | CR8034 | U.S. Pat. No. 8,852,595 SEQ ID NO: 129 | 24373 |
| | region | | | |
| INFL871 | Light chain variable | | U.S. Pat. No. 8,992,929 SEQ ID NO: 2 | 24374 |
| | region | | | |
| INFL872 | Light chain variable | M2e | U.S. Pat. No. 8,420,794 SEQ ID NO: 1 | 24375 |
| | region | | | |
| INFL873 | Light chain variable | | U.S. Pat. No. 8,715,743, US20140275492 SEQ ID | 24376 |
| | region | | NO: 16 | |
| INFL874 | Light chain variable | | U.S. Pat. No. 8,715,743, US20140275492 SEQ ID | 24377 |
| | region | | NO: 19 | |
| INFL875 | Light chain variable | | U.S. Pat. No. 8,715,743, US20140275492 SEQ ID | 24378 |
| | region | | NO: 32 | |
| INFL876 | Light chain variable | anti-1918 influenza | Yu, X., et al., Neutralizing antibodies | 24379 |
| | region | HA Ig | derived from the B cells of 1918 | |
| | | | influenza pandemic survivors; Nature | |
| | | | 455 (7212), 532-536 (2008), NCBI | |
| | | | Accession #ACI04582.1 (121aa) | |
| INFL877 | Light chain variable | anti-1918 influenza | Yu, X., et al., Neutralizing antibodies | 24380 |
| | region | HA Ig | derived from the B cells of 1918 | |
| | | | influenza pandemic survivors; Nature | |
| | | | 455 (7212), 532-536 (2008), NCBI | |
| | | | Accession #ACI04580.1 (118aa) | |
| INFL878 | Light chain variable | 4D20 | Yu, X. et al “Neutralizing antibodies | 24381 |
| | region | | derived from the B cells of 1918 | |
| | | | influenza pandemic survivors”, Nature | |
| | | | 455 (7212), 532-536, NCBI Accession | |
| | | | #ACI04580 | |
| INFL879 | Light chain variable | 2B12 | Yu, X. et al “Neutralizing antibodies | 24382 |
| | region | | derived from the B cells of 1918 | |
| | | | influenza pandemic survivors”, Nature | |
| | | | 455 (7212), 532-536, NCBI Accession | |
| | | | #ABY48869 | |
| INFL880 | Light chain variable | TCN-535 | US20150086555 SEQ ID NO: 180 | 24383 |
| | region | (5246_P19) | | |
| INFL881 | Light chain variable | TCN-536 | US20150086555 SEQ ID NO: 191 | 24384 |
| | region | (5095_N01) | | |
| INFL882 | Light chain variable | TCN-537 | US20150086555 SEQ ID NO: 202 | 24385 |
| | region | (3194_D21) | | |
| INFL883 | Light chain variable | TCN-538 | US20150086555 SEQ ID NO: 214 | 24386 |
| | region | (3206_O17) | | |
| INFL884 | Light chain variable | TCN-539 | US20150086555 SEQ ID NO: 226 | 24387 |
| | region | (5056_A08) | | |
| INFL885 | Light chain variable | TCN-540 | US20150086555 SEQ ID NO: 238 | 24388 |
| | region | (5060_F05) | | |
| INFL886 | Light chain variable | TCN-541 | US20150086555 SEQ ID NO: 250 | 24389 |
| | region | (5062_M11) | | |
| INFL887 | Light chain variable | TCN-542 | US20150086555 SEQ ID NO: 262 | 24390 |
| | region | (5079_A16) | | |
| INFL888 | Light chain variable | TCN-543 | US20150086555 SEQ ID NO: 274 | 24391 |
| | region | (5081_G23) | | |
| INFL889 | Light chain variable | TCN-544 | US20150086555 SEQ ID NO: 286 | 24392 |
| | region | (5082_A19) | | |
| INFL890 | Light chain variable | TCN-545 | US20150086555 SEQ ID NO: 298 | 24393 |
| | region | (5082_I15) | | |
| INFL891 | Light chain variable | TCN-546 | US20150086555 SEQ ID NO: 309 | 24394 |
| | region | (5089_L08) | | |
| INFL892 | Light chain variable | TCN-547 | US20150086555 SEQ ID NO: 320 | 24395 |
| | region | (5092_F11) | | |
| INFL893 | Light chain variable | TCN-548 | US20150086555 SEQ ID NO: 331 | 24396 |
| | region | (5092_P01) | | |
| INFL894 | Light chain variable | TCN-549 | US20150086555 SEQ ID NO: 342 | 24397 |
| | region | (5092_P04) | | |
| INFL895 | Light chain variable | TCN-550 | US20150086555 SEQ ID NO: 353 | 24398 |
| | region | (5096_F06) | | |
| INFL896 | Light chain variable | TCN-551 | US20150086555 SEQ ID NO: 365 | 24399 |
| | region | (5243_D01) | | |
| INFL897 | Light chain variable | TCN-552 | US20150086555 SEQ ID NO: 377 | 24400 |
| | region | (5249_I23) | | |
| INFL898 | Light chain variable | TCN-553 | US20150086555 SEQ ID NO: 389 | 24401 |
| | region | (5261_C18) | | |
| INFL899 | Light chain variable | TCN-554 | US20150086555 SEQ ID NO: 399 | 24402 |
| | region | (5277_M05) | | |
| INFL900 | Light chain variable | TCN-555 | US20150086555 SEQ ID NO: 405 | 24403 |
| | region | (5246_L16) | | |
| INFL901 | Light chain variable | TCN-556 | US20150086555 SEQ ID NO: 415 | 24404 |
| | region | (5089_K12) | | |
| INFL902 | Light chain variable | TCN-557 | US20150086555 SEQ ID NO: 427 | 24405 |
| | region | (5081_A04) | | |
| INFL903 | Light chain variable | TCN-559 | US20150086555 SEQ ID NO: 441 | 24406 |
| | region | (5097_G08) | | |
| INFL904 | Light chain variable | TCN-560 | US20150086555 SEQ ID NO: 453 | 24407 |
| | region | (5084_P10) | | |
| INFL905 | Light chain variable | TCN-564 | US20150086555 SEQ ID NO: 519 | 24408 |
| | region | (5256_A17b) | | |
| INFL906 | Light chain variable | CR8001 | WO2010130636 SEQ ID NO: 4 | 24409 |
| | region | | | |
| INFL907 | Light chain variable | CR8003 | WO2010130636 SEQ ID NO: 8 | 24410 |
| | region | | | |
| INFL908 | Light chain variable | CR8015 | WO2010130636 SEQ ID NO: 12 | 24411 |
| | region | | | |
| INFL909 | Light chain variable | CR8016 | WO2010130636 SEQ ID NO: 16 | 24412 |
| | region | | | |
| INFL910 | Light chain variable | CR8017 | WO2010130636 SEQ ID NO: 20 | 24413 |
| | region | | | |
| INFL911 | Light chain variable | TCN-522 | US20150086555 SEQ ID NO: 40 | 24414 |
| | region | (3212_I12) | | |
| INFL912 | Light chain variable | TCN-521 | US20150086555 SEQ ID NO: 28 | 24415 |
| | region | (3280_D18) | | |
| INFL913 | Light chain variable | TCN-523 | US20150086555 SEQ ID NO: 52 | 24416 |
| | region | (5248_A17), | | |
| | | TCN-533 | | |
| | | (5256_A17a), | | |
| | | TCN-534 | | |
| | | (5249_B02) | | |
| INFL914 | Light chain variable | TCN-563 | US20150086555 SEQ ID NO: 64 | 24417 |
| | region | (5237_B21) | | |
| INFL915 | Light chain variable | TCN-526 | US20150086555 SEQ ID NO: 76 | 24418 |
| | region | (5084_C17) | | |
| INFL916 | Light chain variable | TCN-527 | US20150086555 SEQ ID NO: 88 | 24419 |
| | region | (5086_C06) | | |
| INFL917 | Light chain variable | TCN-528 | US20150086555 SEQ ID NO: 100 | 24420 |
| | region | (5087_P17) | | |
| INFL918 | Light chain variable | TCN-529 | US20150086555 SEQ ID NO: 112 | 24421 |
| | region | (5297_H01) | | |
| INFL919 | Light chain variable | TCN-530 | US20150086555 SEQ ID NO: 124 | 24422 |
| | region | (5248_H10), | | |
| | | TCN-558 | | |
| | | (5248_H10b) | | |
| INFL920 | Light chain variable | TCN-531 | US20150086555 SEQ ID NO: 136 | 24423 |
| | region | (5091_H13) | | |
| INFL921 | Light chain variable | TCN-532 | US20150086555 SEQ ID NO: 148 | 24424 |
| | region | (5262_H18) | | |
| INFL922 | Light chain variable | TCN-534 | US20150086555 SEQ ID NO: 168 | 24425 |
| | region | (5249_B02) | | |
| INFL923 | Light chain variable | TCN-504 | US20150086555 SEQ ID NO: 524 | 24426 |
| | region | (3251_K17) | | |
| INFL924 | Light chain variable | AB1 | US20120093834, WO2009121004 SEQ | 24427 |
| | region | | ID NO: 71 | |
| INFL925 | Light chain variable | AB2 | US20120093834, WO2009121004 SEQ | 24428 |
| | region | | ID NO: 140 | |
| INFL926 | Light chain variable | AB3 | US20120093834, WO2009121004 SEQ | 24429 |
| | region | | ID NO: 81 | |
| INFL927 | Light chain variable | AB4 | US20120093834, WO2009121004 SEQ | 24430 |
| | region | | ID NO: 158 | |
| INFL928 | Light chain variable | AB5 | US20120093834, WO2009121004 SEQ | 24431 |
| | region | | ID NO: 159 | |
| INFL929 | Light chain variable | AB6 | US20120093834, WO2009121004 SEQ | 24432 |
| | region | | ID NO: 160 | |
| INFL930 | Light chain variable | VN04-2 | WO2008033105 SEQ ID NO: 6 | 24433 |
| | region | | | |
| INFL931 | Light chain variable | VN04-3 | WO2008033105 SEQ ID NO: 8 | 24434 |
| | region | | | |
| INFL932 | Light chain variable | 1286-C05 | WO2010132604, US20120128671 SEQ | 24435 |
| | region | | ID NO: 3 | |
| INFL933 | Light chain variable | 1286-C05 | WO2010132604, US20120128671 SEQ | 24436 |
| | region | | ID NO: 4 | |
| INFL934 | Light chain variable | 1286-C05 | WO2010132604, US20120128671 SEQ | 24437 |
| | region | | ID NO: 5 | |
| INFL935 | Light chain variable | 1286-A11 | WO2010132604, US20120128671 SEQ | 24438 |
| | region | | ID NO: 6 | |
| INFL936 | Light chain variable | IA2 | WO2015028478 SEQ ID NO: 3 | 24439 |
| | region mouse IgG | | | |
| INFL937 | Light chain variable | 7B8 | WO2015028478 SEQ ID NO: 5 | 24440 |
| | region mouse IgG | | | |
| INFL938 | Light chain variable | monoclonal | U.S. Pat. No. 8,900,590, US2012039899, Grandea, | 24441 |
| | region, partial | antibody TCN-031 | A. G. et al., Human antibodies reveal a | |
| | | | protective epitope that is highly | |
| | | | conserved among human and nonhuman | |
| | | | influenza A viruses; Proc. Natl. Acad. | |
| | | | Set. U.S.A. 107 (28), 12658-12663 | |
| | | | (2010), NCBI Accession #ADK23871.1 | |
| | | | (106aa) | |
| INFL939 | Light chain variable | monoclonal | Grandea, A. G. et al., Human antibodies | 24442 |
| | region, partial | antibody TCN-032 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23870.1 | |
| | | | (107aa) | |
| INFL940 | Light chain variable | monoclonal | Grandea, A. G. et al., Human antibodies | 24443 |
| | region, partial | antibody 3362_B11 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession # ADK23886.1 | 24444 |
| | | | (107aa) | |
| INFL941 | Light chain variable | monoclonal | Grandea, A. G. et al., Human antibodies | 24445 |
| | region, partial | antibody | reveal a protective epitope that is highly | |
| | | 3260_D19 | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23885.1 | |
| | | | (106aa) | |
| INFL942 | Light chain variable | monoclonal | Grandea, A. G. et al., Human antibodies | 24446 |
| | region, partial | antibody 3253_P10 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession | |
| | | | #ADK23884.1(107aa) | |
| INFL943 | Light chain variable | monoclonal | Grandea, A. G. et al., Human antibodies | 24447 |
| | region, partial | antibody 3248_P18 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23883.1 | |
| | | | (106aa) | |
| INFL944 | Light chain variable | monoclonal | Grandea, A. G. et al., Human antibodies | 24448 |
| | region, partial | antibody 3139_P23 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession | |
| | | | #ADK23882.1(107aa) | |
| INFL945 | Light chain variable | monoclonal | Grandea, A. G. et al., Human antibodies | 24449 |
| | region, partial | antibody 3420_I23 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession | |
| | | | #ADK23881.1(108aa) | |
| INFL946 | Light chain variable | monoclonal | Grandea, A. G. et al., Human antibodies | 24450 |
| | region, partial | antibody 3255_J06 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession | |
| | | | #ADK23880.1(108aa) | |
| INFL947 | Light chain variable | monoclonal | Grandea, A. G. et al., Human antibodies | 24451 |
| | region, partial | antibody 3252_C13 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23879.1 | |
| | | | (108aa) | |
| INFL948 | Light chain variable | monoclonal | Grandea, A. G. et al., Human antibodies | 24452 |
| | region, partial | antibody | reveal a protective epitope that is highly | |
| | | 3136_G05 | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23878.1 | |
| | | | (108aa) | |
| INFL949 | Light chain variable | monoclonal | Grandea, A. G. et al., Human antibodies | 24453 |
| | region, partial | antibody | reveal a protective epitope that is highly | |
| | | 3244_H04 | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23877.1 | |
| | | | (107aa) | |
| INFL950 | Light chain variable | monoclonal | Grandea, A. G. et al., Human antibodies | 24454 |
| | region, partial | antibody | reveal a protective epitope that is highly | |
| | | 3245_O19 | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23876.1 | |
| | | | (107aa) | |
| INFL951 | Light chain variable | monoclonal | Grandea, A. G. et al., Human antibodies | 24455 |
| | region, partial | antibody 3259_J21 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23875.1 | |
| | | | (107aa) | |
| INFL952 | Light chain variable | monoclonal | Grandea, A. G. et al., Human antibodies | 24456 |
| | region, partial | antibody 3243_J07 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23874.1 | |
| | | | (108aa) | |
| INFL953 | Light chain variable | monoclonal | Grandea, A. G. et al., Human antibodies | 24457 |
| | region, partial | antibody 3244_I10 | reveal a protective epitope that is highly | |
| | | | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23873.1 | |
| | | | (108aa) | |
| INFL954 | Light chain variable | monoclonal | Grandea, A. G. et al., Human antibodies | 24458 |
| | region, partial | antibody | reveal a protective epitope that is highly | |
| | | 3241_G23 | conserved among human and nonhuman | |
| | | | influenza A viruses; Proc. Natl. Acad. | |
| | | | Sci. U.S.A. 107 (28), 12658-12663 | |
| | | | (2010), NCBI Accession #ADK23872.1 | |
| | | | (108aa) | |
| INFL955 | Light chain variable | 100F4-LV | Hu, H., et al., A Human Antibody | 24459 |
| | region, partial | | Recognizing a Conserved Epitope of H5 | |
| | | | Hemagglutinin Broadly Neutralizes | |
| | | | Highly Pathogenic Avian Influenza | |
| | | | H5N1 Viruses; J. Virol. 86 (6), 2978- | |
| | | | 2989 (2012), NCBI Accession | |
| | | | #AEL30604.1 (112aa) | |
| INFL956 | Light Chain, Fab | ch65 | Whittle, J. R. et al., Broadly neutralizing | 24460 |
| | Fragment | | human antibody that recognizes the | |
| | | | receptor-binding pocket of influenza | |
| | | | virus hemagglutinin; Proc. Natl. Acad. | |
| | | | Sci. U.S.A. 108 (34), 14216-14221 | |
| | | | (2011), NCBI Accession #3SM5_L | |
| INFL957 | Light chain | 1I20 | WO2010127252, U.S. Pat. No. 8,894,997 SEQ ID | 24461 |
| | | | NO: 6 | |
| INFL958 | Light chain | | WO2010127252, U.S. Pat. No. 8,894,997 SEQ ID | 24462 |
| | | | NO: 12 | |
| INFL959 | Monoclonal antibody | Neutralizing | Wu, Y. et al., A potent broad-spectrum | 24463 |
| | heavy chain | Human | protective human monoclonal antibody | |
| | | Monoclonal | crosslinking two hemagglutinin | |
| | | Antibody With | monomers of influenza A virus; Nat | |
| | | 1968 H3 Ha | Commun 6, 7708 (2015), NCBI | |
| | | | Accession #4UBD_C | |
| INFL960 | Monoclonal antibody | Neutralizing | Wu, Y. et al., A potent broad-spectrum | 24464 |
| | light chain | Human | protective human monoclonal antibody | |
| | | Monoclonal | crosslinking two hemagglutinin | |
| | | Antibody With | monomers of influenza A virus; Nat | |
| | | 1968 H3 Ha | Commun 6, 7708 (2015), NCBI | |
| | | | Accession #4UBD_D | |
| INFL961 | Mutated heavy chain | 8G9 mutated | U.S. Pat. No. 8,603,467 SEQ ID NO: 42 | 24465 |
| | variable | | | |
| INFL962 | Mutated heavy chain | 13D4 mutated | U.S. Pat. No. 8,603,467 SEQ ID NO: 46 | 24466 |
| | variable (VH-LV) | | | |
| INFL963 | Mutated heavy chain | 13D4 mutated | U.S. Pat. No. 8,603,467 SEQ ID NO: 44 | 24467 |
| | variable (VH-SV) | | | |
| INFL964 | Nanobody | 202-C8 | US20110182897, WO2009147248 SEQ | 24468 |
| | | | ID NO: 138 | |
| INFL965 | Nanobody | 203-B12 | US20110182897, WO2009147248 SEQ | 24469 |
| | | | ID NO: 2439 | |
| INFL966 | Nanobody | 203-H9 | US20110182897, WO2009147248 SEQ | 24470 |
| | | | ID NO: 2445 | |
| INFL967 | Scfv | JM7_B-G7 | WO2012072788 SEQ ID NO: 7 | 24471 |
| INFL968 | Scfv | JM7_S-F8 | WO2012072788 SEQ ID NO: 15 | 24472 |
| INFL969 | Scfv | JM7JH-F1 | WO2012072788 SEQ ID NO: 17 | 24473 |
| INFL970 | Scfv | JM7_S-A9 | WO2012072788 SEQ ID NO: 19 | 24474 |
| INFL971 | Scfv | JM7_S-A10 | WO2012072788 SEQ ID NO: 21 | 24475 |
| INFL972 | Scfv | JM7_B-H | WO2012072788 SEQ ID NO: 23 | 24476 |
| INFL973 | Scfv | JM6_SC-H1 | WO2012072788 SEQ ID NO: 25 | 24477 |
| INFL974 | Scfv | jM6_SC_D3 | WO2012072788 SEQ ID NO: 27 | 24478 |
| INFL975 | Scfv | H2526 | Schmidt, A. G. et al., Viral receptor- | 24479 |
| | | | binding site antibodies with diverse | |
| | | | germline origins; Cell 161 (5), 1026- | |
| | | | 1034 (2015), NCBI Accession #4YJZ_L | |
| INFL976 | Scfv fragment | AVC4 | WO2010040572A2 FIG. 6 | 24480 |
| INFL977 | Scfv fragment | AVD1 | WO2010040572A2 FIG. 8 | 24481 |
| INFL978 | Scfv fragment | AVE2 | WO2010040572A2 FIG. 10 | 24482 |
| INFL979 | Scfv fragment | AVA6 | WO2010040572A2 FIG. 12 | 24483 |
| INFL980 | Scfv fragment | AVG4 | WO2010040572A2 FIG. 14 | 24484 |
| INFL981 | Scfv heavy chain | SC06-141 | US20150104459 SEQ ID NO: 309 | 24485 |
| | variable region | | | |
| INFL982 | Scfv heavy chain | SC06-255 | US20150104459 SEQ ID NO: 313 | 24486 |
| | variable region | | | |
| INFL983 | Scfv heavy chain | SC06-257 | US20150104459 SEQ ID NO: 317 | 24487 |
| | variable region | | | |
| INFL984 | Scfv heavy chain | SC6-260 | US20150104459 SEQ ID NO: 321 | 24488 |
| | variable region | | | |
| INFL985 | Scfv heavy chain | SC06-261 | US20150104459 SEQ ID NO: 325 | 24489 |
| | variable region | | | |
| INFL986 | Scfv heavy chain | SC06-262 | US20150104459 SEQ ID NO: 329 | 24490 |
| | variable region | | | |
| INFL987 | Scfv heavy chain | SC06-268 | US20150104459 SEQ ID NO: 333 | 24491 |
| | variable region | | | |
| INFL988 | Scfv heavy chain | SC06-272 | US20150104459 SEQ ID NO: 337 | 24492 |
| | variable region | | | |
| INFL989 | Scfv heavy chain | SC06-296 | US20150104459 SEQ ID NO: 341 | 24493 |
| | variable region | | | |
| INFL990 | Scfv heavy chain | SC06-301 | US20150104459 SEQ ID NO: 345 | 24494 |
| | variable region | | | |
| INFL991 | Scfv heavy chain | SC06-307 | US20150104459 SEQ ID NO: 349 | 24495 |
| | variable region | | | |
| INFL992 | Scfv heavy chain | SC06-310 | US20150104459 SEQ ID NO: 353 | 24496 |
| | variable region | | | |
| INFL993 | Scfv heavy chain | SC06-314 | US20150104459 SEQ ID NO: 357 | 24497 |
| | variable region | | | |
| INFL994 | Scfv heavy chain | SC06-323 | US20150104459 SEQ ID NO: 361 | 24498 |
| | variable region | | | |
| INFL995 | Scfv heavy chain | SC06-325 | US20150104459 SEQ ID NO: 365 | 24499 |
| | variable region | | | |
| INFL996 | Scfv heavy chain | SC06-327 | US20150104459 SEQ ID NO: 369 | 24500 |
| | variable region | | | |
| INFL997 | Scfv heavy chain | SC06-328 | US20150104459 SEQ ID NO: 373 | 24501 |
| | variable region | | | |
| INFL998 | Scfv heavy chain | SC06-329 | US20150104459 SEQ ID NO: 377 | 24502 |
| | variable region | | | |
| INFL999 | Scfv heavy chain | SC06-331 | US20150104459 SEQ ID NO: 381 | 24503 |
| | variable region | | | |
| INFL1000 | Scfv heavy chain | SC06-332 | US20150104459 SEQ ID NO: 385 | 24504 |
| | variable region | | | |
| INFL1001 | Scfv heavy chain | SC06-334 | US20150104459 SEQ ID NO: 389 | 24505 |
| | variable region | | | |
| INFL1002 | Scfv heavy chain | SC06-336 | US20150104459 SEQ ID NO: 393 | 24506 |
| | variable region | | | |
| INFL1003 | Scfv heavy chain | SC06-339 | US20150104459 SEQ ID NO: 397 | 24507 |
| | variable region | | | |
| INFL1004 | Scfv heavy chain | SC06-342 | US20150104459 SEQ ID NO: 401 | 24508 |
| | variable region | | | |
| INFL1005 | Scfv heavy chain | SC06-343 | US20150104459 SEQ ID NO: 405 | 24509 |
| | variable region | | | |
| INFL1006 | Scfv heavy chain | SC06-344 | US20150104459 SEQ ID NO: 409 | 24510 |
| | variable region | | | |
| INFL1007 | Scfv heavy chain | CR6255 | US20150104459 SEQ ID NO: 417 | 24511 |
| | variable region | | | |
| INFL1008 | Scfv heavy chain | CR6257 | US20150104459 SEQ ID NO: 423 | 24512 |
| | variable region | | | |
| INFL1009 | Scfv heavy chain | CR6260 | US20150104459 SEQ ID NO: 429 | 24513 |
| | variable region | | | |
| INFL1010 | Scfv heavy chain | CR6261 | US20150104459 SEQ ID NO: 435 | 24514 |
| | variable region | | | |
| INFL1011 | Scfv heavy chain | CR6262 | US20150104459 SEQ ID NO: 441 | 24515 |
| | variable region | | | |
| INFL1012 | Scfv heavy chain | CR6268 | US20150104459 SEQ ID NO: 447 | 24516 |
| | variable region | | | |
| INFL1013 | Scfv heavy chain | CR6272 | US20150104459 SEQ ID NO: 453 | 24517 |
| | variable region | | | |
| INFL1014 | Scfv heavy chain | CR696 | US20150104459 SEQ ID NO: 459 | 24518 |
| | variable region | | | |
| INFL1015 | Scfv heavy chain | CR6301 | US20150104459 SEQ ID NO: 465 | 24519 |
| | variable region | | | |
| INFL1016 | Scfv heavy chain | CR6307 | US20150104459 SEQ ID NO: 471 | 24520 |
| | variable region | | | |
| INFL1017 | Scfv heavy chain | CR6310 | US20150104459 SEQ ID NO: 477 | 24521 |
| | variable region | | | |
| INFL1018 | Scfv heavy chain | CR6314 | US20150104459 SEQ ID NO: 483 | 24522 |
| | variable region | | | |
| INFL1019 | Scfv heavy chain | CR6323 | US20150104459 SEQ ID NO: 489 | 24523 |
| | variable region | | | |
| INFL1020 | Scfv heavy chain | CR6325 | US20150104459 SEQ ID NO: 495 | 24524 |
| | variable region | | | |
| INFL1021 | Scfv heavy chain | CR6327 | US20150104459 SEQ ID NO: 501 | 24525 |
| | variable region | | | |
| INFL1022 | Scfv heavy chain | CR6328 | US20150104459 SEQ ID NO: 507 | 24526 |
| | variable region | | | |
| INFL1023 | Scfv heavy chain | CR6329 | US20150104459 SEQ ID NO: 513 | 24527 |
| | variable region | | | |
| INFL1024 | Scfv heavy chain | CR6331 | US20150104459 SEQ ID NO: 519 | 24528 |
| | variable region | | | |
| INFL1025 | Scfv heavy chain | CR6332 | US20150104459 SEQ ID NO: 525 | 24529 |
| | variable region | | | |
| INFL1026 | Scfv heavy chain | CR6334 | US20150104459 SEQ ID NO: 531 | 24530 |
| | variable region | | | |
| INFL1027 | Scfv heavy chain | CR6336 | US20150104459 SEQ ID NO: 537 | 24531 |
| | variable region | | | |
| INFL1028 | Scfv heavy chain | CR6339 | US20150104459 SEQ ID NO: 543 | 24532 |
| | variable region | | | |
| INFL1029 | Scfv heavy chain | CR6342 | US20150104459 SEQ ID NO: 550 | 24533 |
| | variable region | | | |
| INFL1030 | Scfv heavy chain | CR6343 | US20150104459 SEQ ID NO: 556 | 24534 |
| | variable region | | | |
| INFL1031 | Scfv heavy chain | CR6344 | US20150104459 SEQ ID NO: 562 | 24535 |
| | variable region | | | |
| INFL1032 | Scfv light chain | SC06-141 | US20150104459 SEQ ID NO: 310 | 24536 |
| | variable region | | | |
| INFL1033 | Scfv light chain | SC06-255 | US20150104459 SEQ ID NO: 314 | 24537 |
| | variable region | | | |
| INFL1034 | Scfv light chain | SC06-257 | US20150104459 SEQ ID NO: 318 | 24538 |
| | variable region | | | |
| INFL1035 | Scfv light chain | SC6-260 | US20150104459 SEQ ID NO: 322 | 24539 |
| | variable region | | | |
| INFL1036 | Scfv light chain | SC06-261 | US20150104459 SEQ ID NO: 326 | 24540 |
| | variable region | | | |
| INFL1037 | Scfv light chain | SC06-262 | US20150104459 SEQ ID NO: 330 | 24541 |
| | variable region | | | |
| INFL1038 | Scfv light chain | SC06-268 | US20150104459 SEQ ID NO: 334 | 24542 |
| | variable region | | | |
| INFL1039 | Scfv light chain | SC06-272 | US20150104459 SEQ ID NO: 338 | 24543 |
| | variable region | | | |
| INFL1040 | Scfv light chain | SC06-296 | US20150104459 SEQ ID NO: 342 | 24544 |
| | variable region | | | |
| INFL1041 | Scfv light chain | SC06-301 | US20150104459 SEQ ID NO: 346 | 24545 |
| | variable region | | | |
| INFL1042 | Scfv light chain | SC06-307 | US20150104459 SEQ ID NO: 350 | 24546 |
| | variable region | | | |
| INFL1043 | Scfv light chain | SC06-310 | US20150104459 SEQ ID NO: 354 | 24547 |
| | variable region | | | |
| INFL1044 | Scfv light chain | SC06-314 | US20150104459 SEQ ID NO: 358 | 24548 |
| | variable region | | | |
| INFL1045 | Scfv light chain | SC06-323 | US20150104459 SEQ ID NO: 362 | 24549 |
| | variable region | | | |
| INFL1046 | Scfv light chain | SC06-325 | US20150104459 SEQ ID NO: 366 | 24550 |
| | variable region | | | |
| INFL1047 | Scfv light chain | SC06-327 | US20150104459 SEQ ID NO: 370 | 24551 |
| | variable region | | | |
| INFL1048 | Scfv light chain | SC06-328 | US20150104459 SEQ ID NO: 374 | 24552 |
| | variable region | | | |
| INFL1049 | Scfv light chain | SC06-329 | US20150104459 SEQ ID NO: 378 | 24553 |
| | variable region | | | |
| INFL1050 | Scfv light chain | SC06-331 | US20150104459 SEQ ID NO: 382 | 24554 |
| | variable region | | | |
| INFL1051 | Scfv light chain | SC06-332 | US20150104459 SEQ ID NO: 386 | 24555 |
| | variable region | | | |
| INFL1052 | Scfv light chain | SC06-334 | US20150104459 SEQ ID NO: 390 | 24556 |
| | variable region | | | |
| INFL1053 | Scfv light chain | SC06-336 | US20150104459 SEQ ID NO: 394 | 24557 |
| | variable region | | | |
| INFL1054 | Scfv light chain | SC06-339 | US20150104459 SEQ ID NO: 398 | 24558 |
| | variable region | | | |
| INFL1055 | Scfv light chain | SC06-342 | US20150104459 SEQ ID NO: 402 | 24559 |
| | variable region | | | |
| INFL1056 | Scfv light chain | SC06-343 | US20150104459 SEQ ID NO: 406 | 24560 |
| | variable region | | | |
| INFL1057 | Scfv light chain | SC06-344 | US20150104459 SEQ ID NO: 410 | 24561 |
| | variable region | | | |
| INFL1058 | Scfv light chain | CR6141 | US20150104459 SEQ ID NO: 414 | 24562 |
| | variable region | | | |
| INFL1059 | Scfv light chain | CR6255 | US20150104459 SEQ ID NO: 420 | 24563 |
| | variable region | | | |
| INFL1060 | Scfv light chain | CR6257 | US20150104459 SEQ ID NO: 426 | 24564 |
| | variable region | | | |
| INFL1061 | Scfv light chain | CR6260 | US20150104459 SEQ ID NO: 432 | 24565 |
| | variable region | | | |
| INFL1062 | Scfv light chain | CR6261 | US20150104459 SEQ ID NO: 438 | 24566 |
| | variable region | | | |
| INFL1063 | Scfv light chain | CR6262 | US20150104459 SEQ ID NO: 444 | 24567 |
| | variable region | | | |
| INFL1064 | Scfv light chain | CR6268 | US20150104459 SEQ ID NO: 450 | 24568 |
| | variable region | | | |
| INFL1065 | Scfv light chain | CR6272 | US20150104459 SEQ ID NO: 456 | 24569 |
| | variable region | | | |
| INFL1066 | Scfv light chain | CR696 | US20150104459 SEQ ID NO: 462 | 24570 |
| | variable region | | | |
| INFL1067 | Scfv light chain | CR6301 | US20150104459 SEQ ID NO: 468 | 24571 |
| | variable region | | | |
| INFL1068 | Scfv light chain | CR6307 | US20150104459 SEQ ID NO: 474 | 24572 |
| | variable region | | | |
| INFL1069 | Scfv light chain | CR6310 | US20150104459 SEQ ID NO: 480 | 24573 |
| | variable region | | | |
| INFL1070 | Scfv light chain | CR6314 | US20150104459 SEQ ID NO: 486 | 24574 |
| | variable region | | | |
| INFL1071 | Scfv light chain | CR6323 | US20150104459 SEQ ID NO: 492 | 24575 |
| | variable region | | | |
| INFL1072 | Scfv light chain | CR6325 | US20150104459 SEQ ID NO: 498 | 24576 |
| | variable region | | | |
| INFL1073 | Scfv light chain | CR6327 | US20150104459 SEQ ID NO: 504 | 24577 |
| | variable region | | | |
| INFL1074 | Scfv light chain | CR6328 | US20150104459 SEQ ID NO: 510 | 24578 |
| | variable region | | | |
| INFL1075 | Scfv light chain | CR6329 | US20150104459 SEQ ID NO: 516 | 24579 |
| | variable region | | | |
| INFL1076 | Scfv light chain | CR6331 | US20150104459 SEQ ID NO: 522 | 24580 |
| | variable region | | | |
| INFL1077 | Scfv light chain | CR6332 | US20150104459 SEQ ID NO: 528 | 24581 |
| | variable region | | | |
| INFL1078 | Scfv light chain | CR6334 | US20150104459 SEQ ID NO: 534 | 24582 |
| | variable region | | | |
| INFL1079 | Scfv light chain | CR6336 | US20150104459 SEQ ID NO: 540 | 24583 |
| | variable region | | | |
| INFL1080 | Scfv light chain | CR6339 | US20150104459 SEQ ID NO: 547 | 24584 |
| | variable region | | | |
| INFL1081 | Scfv light chain | CR6342 | US20150104459 SEQ ID NO: 553 | 24585 |
| | variable region | | | |
| INFL1082 | Scfv light chain | CR6343 | US20150104459 SEQ ID NO: 559 | 24586 |
| | variable region | | | |
| INFL1083 | Scfv light chain | CR6344 | US20150104459 SEQ ID NO: 565 | 24587 |
| | variable region | | | |
| INFL1084 | Vhch antibody | 641 I-9 | Schmidt, A. G. et al., Viral receptor- | 24588 |
| | | | binding site antibodies with diverse | |
| | | | germline origins; Cell 161 (5), 1026- | |
| | | | 1034 (2015), NCBI Accession #4YK4_Z | |
| INFL1085 | Vlcl antibody | 641 I-9 | Schmidt, A. G. et al., Viral receptor- | 24589 |
| | | | binding site antibodies with diverse | |
| | | | germline origins; Cell 161 (5), 1026- | |
| | | | 1034 (2015), NCBI Accession #4YK4_Y | |

In some embodiments, the payload region of the viral particle comprises one or more nucleic acid sequences, fragments or variants thereof or encodes one or more polypeptides, fragments or variants thereof described in U.S. Pat. Nos. 8,003,106 and 8,540,995, International Patent Publication No. WO2015028478, WO2012045001, US Publication No. US20150239960 and US20130251715, the contents of each of which are herein incorporated by reference in their entirety, against influenza.

In some embodiments, the payload region of the viral particle comprises one or more nucleic acid sequences encoding one or more of the payload antibody polypeptides listed in Table 33 against Respiratory Syncytial Virus (RSV1-RSV1088; SEQ ID NO: 24581-25668).

TABLE 33
Antibodies against Repsiratory Syncytial Virus

| Antibody | | | | SEQ ID |
|---|---|---|---|---|
| No. | Description | Antibody Name | Reference Information | NO |
| RSV1 | Heavy chain variable, F | clone 888 | US20110189171; U.S. Pat. No. 7,879,329 | 24581 |
| | and G Proteins | | SEQ ID NO: 43 | |
| RSV2 | Heavy chain variable, F | mAb 824 | US20110189171; U.S. Pat. No. 7,879,329 | 24582 |
| | and G Proteins | | SEQ ID NO: 178 | |
| RSV3 | Heavy chain variable, F | clone 735 | US20110189171; U.S. Pat. No. 7,879,329 | 24583 |
| | and G Proteins | | SEQ ID NO: 1 | |
| RSV4 | Heavy chain variable, F | clone 736 | US20110189171; U.S. Pat. No. 7,879,329 | 24584 |
| | and G Proteins | | SEQ ID NO: 2 | |
| RSV5 | Heavy chain variable, F | clone 744 | US20110189171; U.S. Pat. No. 7,879,329 | 24585 |
| | and G Proteins | | SEQ ID NO: 3 | |
| RSV6 | Heavy chain variable, F | clone 793 | US20110189171; U.S. Pat. No. 7,879,329 | 24586 |
| | and G Proteins | | SEQ ID NO: 4 | |
| RSV7 | Heavy chain variable, F | clone 795 | US20110189171; U.S. Pat. No. 7,879,329 | 24587 |
| | and G Proteins | | SEQ ID NO: 5 | |
| RSV8 | Heavy chain variable, F | clone 796 | US20110189171; U.S. Pat. No. 7,879,329 | 24588 |
| | and G Proteins | | SEQ ID NO: 6 | |
| RSV9 | Heavy chain variable, F | clone 799 | US20110189171; U.S. Pat. No. 7,879,329 | 24589 |
| | and G Proteins | | SEQ ID NO: 7 | |
| RSV10 | Heavy chain variable, F | clone 800 | US20110189171; U.S. Pat. No. 7,879,329 | 24590 |
| | and G Proteins | | SEQ ID NO: 8 | |
| RSV11 | Heavy chain variable, F | clone 801 | US20110189171; U.S. Pat. No. 7,879,329 | 24591 |
| | and G Proteins | | SEQ ID NO: 9 | |
| RSV12 | Heavy chain variable, F | clone 804 | US20110189171; U.S. Pat. No. 7,879,329 | 24592 |
| | and G Proteins | | SEQ ID NO: 10 | |
| RSV13 | Heavy chain variable, F | clone 810 | US20110189171; U.S. Pat. No. 7,879,329 | 24593 |
| | and G Proteins | | SEQ ID NO: 11 | |
| RSV14 | Heavy chain variable, F | clone 811 | US20110189171; U.S. Pat. No. 7,879,329 | 24594 |
| | and G Proteins | | SEQ ID NO: 12 | |
| RSV15 | Heavy chain variable, F | clone 812 | US20110189171; U.S. Pat. No. 7,879,329 | 24595 |
| | and G Proteins | | SEQ ID NO: 13 | |
| RSV16 | Heavy chain variable, F | clone 814 | US20110189171; U.S. Pat. No. 7,879,329 | 24596 |
| | and G Proteins | | SEQ ID NO: 14 | |
| RSV17 | Heavy chain variable, F | clone 816 | US20110189171; U.S. Pat. No. 7,879,329 | 24597 |
| | and G Proteins | | SEQ ID NO: 15 | |
| RSV18 | Heavy chain variable, F | clone 817 | US20110189171; U.S. Pat. No. 7,879,329 | 24598 |
| | and G Proteins | | SEQ ID NO: 16 | |
| RSV19 | Heavy chain variable, F | clone 818 | US20110189171; U.S. Pat. No. 7,879,329 | 24599 |
| | and G Proteins | | SEQ ID NO: 17 | |
| RSV20 | Heavy chain variable, F | clone 819 | US20110189171; U.S. Pat. No. 7,879,329 | 24600 |
| | and G Proteins | | SEQ ID NO: 18 | |
| RSV21 | Heavy chain variable, F | clone 824 | US20110189171; U.S. Pat. No. 7,879,329 | 24601 |
| | and G Proteins | | SEQ ID NO: 19 | |
| RSV22 | Heavy chain variable, F | clone 825 | US20110189171; U.S. Pat. No. 7,879,329 | 24602 |
| | and G Proteins | | SEQ ID NO: 20 | |
| RSV23 | Heavy chain variable, F | clone 827 | US20110189171; U.S. Pat. No. 7,879,329 | 24603 |
| | and G Proteins | | SEQ ID NO: 21 | |
| RSV24 | Heavy chain variable, F | clone 829 | US20110189171; U.S. Pat. No. 7,879,329 | 24604 |
| | and G Proteins | | SEQ ID NO: 22 | |
| RSV25 | Heavy chain variable, F | clone 830 | US20110189171; U.S. Pat. No. 7,879,329 | 24605 |
| | and G Proteins | | SEQ ID NO: 23 | |
| RSV26 | Heavy chain variable, F | clone 831 | US20110189171; U.S. Pat. No. 7,879,329 | 24606 |
| | and G Proteins | | SEQ ID NO: 24 | |
| RSV27 | Heavy chain variable, F | clone 835 | US20110189171; U.S. Pat. No. 7,879,329 | 24607 |
| | and G Proteins | | SEQ ID NO: 25 | |
| RSV28 | Heavy chain variable, F | clone 838 | US20110189171; U.S. Pat. No. 7,879,329 | 24608 |
| | and G Proteins | | SEQ ID NO: 26 | |
| RSV29 | Heavy chain variable, F | clone 841 | US20110189171; U.S. Pat. No. 7,879,329 | 24609 |
| | and G Proteins | | SEQ ID NO: 27 | |
| RSV30 | Heavy chain variable, F | clone 853 | US20110189171; U.S. Pat. No. 7,879,329 | 24610 |
| | and G Proteins | | SEQ ID NO: 28 | |
| RSV31 | Heavy chain variable, F | clone 855 | US20110189171; U.S. Pat. No. 7,879,329 | 24611 |
| | and G Proteins | | SEQ ID NO: 29 | |
| RSV32 | Heavy chain variable, F | clone 856 | US20110189171; U.S. Pat. No. 7,879,329 | 24612 |
| | and G Proteins | | SEQ ID NO: 30 | |
| RSV33 | Heavy chain variable, F | clone 857 | US20110189171; U.S. Pat. No. 7,879,329 | 24613 |
| | and G Proteins | | SEQ ID NO: 31 | |
| RSV34 | Heavy chain variable, F | clone 858 | US20110189171; U.S. Pat. No. 7,879,329 | 24614 |
| | and G Proteins | | SEQ ID NO: 32 | |
| RSV35 | Heavy chain variable, F | clone 859 | US20110189171; U.S. Pat. No. 7,879,329 | 24615 |
| | and G Proteins | | SEQ ID NO: 33 | |
| RSV36 | Heavy chain variable, F | clone 861 | US20110189171; U.S. Pat. No. 7,879,329 | 24616 |
| | and G Proteins | | SEQ ID NO: 34 | |
| RSV37 | Heavy chain variable, F | clone 863 | US20110189171; U.S. Pat. No. 7,879,329 | 24617 |
| | and G Proteins | | SEQ ID NO: 35 | |
| RSV38 | Heavy chain variable, F | clone 868 | US20110189171; U.S. Pat. No. 7,879,329 | 24618 |
| | and G Proteins | | SEQ ID NO: 36 | |
| RSV39 | Heavy chain variable, F | clone 870 | US20110189171; U.S. Pat. No. 7,879,329 | 24619 |
| | and G Proteins | | SEQ ID NO: 37 | |
| RSV40 | Heavy chain variable, F | clone 871 | US20110189171; U.S. Pat. No. 7,879,329 | 24620 |
| | and G Proteins | | SEQ ID NO: 38 | |
| RSV41 | Heavy chain variable, F | clone 880 | US20110189171; U.S. Pat. No. 7,879,329 | 24621 |
| | and G Proteins | | SEQ ID NO: 39 | |
| RSV42 | Heavy chain variable, F | clone 881 | US20110189171; U.S. Pat. No. 7,879,329 | 24622 |
| | and G Proteins | | SEQ ID NO: 40 | |
| RSV43 | Heavy chain variable, F | clone 884 | US20110189171; U.S. Pat. No. 7,879,329 | 24623 |
| | and G Proteins | | SEQ ID NO: 41 | |
| RSV44 | Heavy chain variable, F | clone 886 | US20110189171; U.S. Pat. No. 7,879,329 | 24624 |
| | and G Proteins | | SEQ ID NO: 42 | |
| RSV45 | Heavy chain variable, F | clone 894 | US20110189171; U.S. Pat. No. 7,879,329 | 24625 |
| | and G Proteins | | SEQ ID NO: 44 | |
| RSV46 | heavy chain variable, F | 3210 variant 1 | WO2013140247 SEQ ID NO: | 24626 |
| | protein of RSV, MPV, or | | 13 | |
| | PVM | | | |
| RSV47 | heavy chain variable, F | 3210 variant 2, | WO2013140247 SEQ ID NO: | 24627 |
| | protein of RSV, MPV, or | 3210 variant 3, | 17 | |
| | PVM | 3210 variant 6 | | |
| RSV48 | heavy chain variable, F | 2430 variant 1 | WO2013140247 SEQ ID NO: | 24628 |
| | protein of RSV, MPV, or | | 29 | |
| | PVM | | | |
| RSV49 | heavy chain variable, F | 2430 variant 2, | WO2013140247 SEQ ID NO: | 24629 |
| | protein of RSV, MPV, or | 2430 variant 5 | 33 | |
| | PVM | | | |
| RSV50 | heavy chain variable, F | 3210 variant 4, | WO2013140247 SEQ ID NO: | 24630 |
| | protein of RSV, MPV, or | 3210 variant 5 | 49 | |
| | PVM | | | |
| RSV51 | heavy chain variable, F | 2430 variant 3, | WO2013140247 SEQ ID NO: | 24631 |
| | protein of RSV, MPV, or | 2430 variant 4 | 59 | |
| | PVM | | | |
| RSV52 | Heavy chain variable, | | US20140093501 SEQ ID NO: | 24632 |
| | CDR Grafted, F Protein, | | 31 | |
| RSV53 | Heavy chain, F Protein | AM22 | U.S. Pat. No. 8,568,726 SEQ ID NO: 16 | 24633 |
| RSV54 | Heavy chain, F Protein | RSVF2-5 | U.S. Pat. No. 8,221,759 SEQ ID NO: 1 | 24634 |
| RSV55 | Heavy chain, F Protein | | EP1259547; U.S. Pat. No. 8,153,133 | 24635 |
| | | | SEQ ID NO: 4 | |
| RSV56 | Heavy chain, F Protein | MEDI- | EP1259547; U.S. Pat. No. 8,153,133 SEQ | 24636 |
| | | 493/Pavilizumab- | ID NO: 2 | |
| | | N-VL (Brand name | | |
| | | Synagis) | | |
| RSV57 | Heavy chain, F Protein | | EP1259547; U.S. Pat. No. 8,153,133 SEQ | 24637 |
| | | | ID NO: 36 | |
| RSV58 | Heavy chain, F Protein | clone 18 | EP1259547; U.S. Pat. No. 8,153,133 SEQ | 24638 |
| | | | ID NO: 37 | |
| RSV59 | Heavy chain, F Protein | clone 19 | EP1259547; U.S. Pat. No. 8,153,133 SEQ | 24639 |
| | | | ID NO: 39 | |
| RSV60 | Heavy chain, F Protein | clone 20 | EP1259547; U.S. Pat. No. 8,153,133 SEQ | 24640 |
| | | | ID NO: 41 | |
| RSV61 | Heavy chain, F Protein | clone 21 | EP1259547; U.S. Pat. No. 8,153,133 SEQ | 24641 |
| | | | ID NO: 43 | |
| RSV62 | Heavy chain, F Protein | clone 22 | EP1259547; U.S. Pat. No. 8,153,133 SEQ | 24642 |
| | | | ID NO: 45 | |
| RSV63 | Heavy chain, F Protein | clone 23 | EP1259547; U.S. Pat. No. 8,153,133 SEQ | 24643 |
| | | | ID NO: 47 | |
| RSV64 | Heavy chain, F Protein | clone 24 | EP1259547; U.S. Pat. No. 8,153,133 SEQ | 24644 |
| | | | ID NO: 49 | |
| RSV65 | Heavy chain, F Protein | clone 25 | EP1259547; U.S. Pat. No. 8,153,133 SEQ | 24645 |
| | | | ID NO: 51 | |
| RSV66 | Heavy chain, F Protein | clone 26 | EP1259547; U.S. Pat. No. 8,153,133 SEQ | 24646 |
| | | | ID NO: 53 | |
| RSV67 | Heavy chain variable | | US20140093501 SEQ ID NO: | 24647 |
| | region, F Protein | | 17 | |
| RSV68 | Heavy chain variable | MAb1308F | US20140093501 SEQ ID NO: | 24648 |
| | region, F Protein | | 18 | |
| RSV69 | Heavy chain variable | huCOR | US20140093501 SEQ ID NO: | 24649 |
| | region, F Protein | | 30 | |
| RSV70 | Heavy chain variable | M.Ab1129 | US20140093501 SEQ ID NO: | 24650 |
| | region, F Protein | | 32 | |
| RSV71 | Heavy chain variable | RSV G8 | U.S. Pat. No. 7,867,497 SEQ ID NO: 2 | 24651 |
| | region, F Protein | | | |
| RSV72 | Heavy chain variable | Clone 1 | US20120135006 SEQ ID NO: | 24652 |
| | region, F Protein | | 18 | |
| RSV73 | Heavy chain variable | Clone 2 | US20120135006 SEQ ID NO: | 24653 |
| | region, F Protein | | 20 | |
| RSV74 | Heavy chain variable | Clone 3 | US20120135006 SEQ ID NO: | 24654 |
| | region, F Protein | | 22 | |
| RSV75 | Heavy chain variable | Clone 22 | US20120135006 SEQ ID NO: | 24655 |
| | region, F Protein | | 24 | |
| RSV76 | Heavy chain variable | Clone 23 | US20120135006 SEQ ID NO: | 24656 |
| | region, F Protein | | 26 | |
| RSV77 | Heavy chain variable | RSV13-9 | WO2009088159 SEQ ID NO: 4 | 24657 |
| | region, F Protein | | | |
| RSV78 | HV3 heavy chain | | US20140093501 SEQ ID NO: | 24658 |
| | variable, F Protein | | 16 | |
| RSV79 | Constant heavy region, F | B4HuVK | EP636182; WO1993020210; | 24659 |
| | protein | | SEQ ID NO: 6 | |
| RSV80 | Constant heavy region, F | B13/B14HuVK | EP636182; WO1993020210; | 24660 |
| | protein | | SEQ ID NO: 8 | |
| RSV81 | Heavy chain, F protein | 58c5 | US20140044719 SEQ ID NO: 1 | 24661 |
| RSV82 | Heavy chain, F protein | sc5 | US20140044719 SEQ ID NO: 9 | 24662 |
| RSV83 | Heavy chain, F protein | | US20110027294 SEQ ID NO: | 24663 |
| | | | 74 | |
| RSV84 | Heavy chain, F protein | | US20110027294 SEQ ID NO: | 24664 |
| | | | 75 | |
| RSV85 | Heavy chain, F protein | | US20110027294 SEQ ID NO: | 24665 |
| | | | 76 | |
| RSV86 | Heavy chain, F protein | | US20110027294 SEQ ID NO: | 24666 |
| | | | 77 | |
| RSV87 | Heavy chain, F protein | | US20110027294 SEQ ID NO: | 24667 |
| | | | 78 | |
| RSV88 | Heavy chain, F protein | | US20110027294 SEQ ID NO: | 24668 |
| | | | 79 | |
| RSV89 | Heavy chain, F protein | | US20110027294 SEQ ID NO: | 24669 |
| | | | 80 | |
| RSV90 | Heavy chain, F protein | Gλ-1 | US20050175986 SEQ ID NO: 5 | 24670 |
| RSV91 | Heavy chain, F protein | A construct | US20050175986 SEQ ID NO: 7 | 24671 |
| RSV92 | Heavy chain, F protein | B construct | US20050175986 SEQ ID NO: 8 | 24672 |
| RSV93 | Heavy chain, F protein | hu19A | US20050019758; | 24673 |
| | | | WO1998019704 SEQ ID NO: 5 | |
| RSV94 | Heavy chain, F protein | hu19B | US20050019758; | 24674 |
| | | | WO1998019704 SEQ ID NO: 6 | |
| RSV95 | Heavy chain, F protein | hu19C | US20050019758; | 24675 |
| | | | WO1998019704 SEQ ID NO: 7 | |
| RSV96 | Heavy chain, F protein | hu19D | US20050019758; | 24676 |
| | | | WO1998019704 SEQ ID NO: 8 | |
| RSV97 | Heavy chain, F protein | B4HuVH | EP636182; WO1993020210; | 24677 |
| | | | SEQ ID NO: 5 | |
| RSV98 | Heavy chain, F protein | B13/B14HuVK | EP636182; WO1993020210; | 24678 |
| | | | SEQ ID NO: 7 | |
| RSV99 | Heavy chain, F protein | RSV19 | EP636182; WO1993020210; | 24679 |
| | | | SEQ ID NO: 10 | |
| RSV100 | Heavy chain, F protein | | WO19922004381 | 24680 |
| RSV101 | Heavy chain, F protein | | WO19922004381 | 24681 |
| RSV102 | Heavy chain variable | P1212 | US20140044719 SEQ ID NO: | 24682 |
| | region, F Protein | | 122 | |
| RSV103 | Heavy chain variable | P12f4 | US20140044719 SEQ ID NO: | 24683 |
| | region, F Protein | | 131 | |
| RSV104 | Heavy chain variable | P11d4 | US20140044719 SEQ ID NO: | 24684 |
| | region, F Protein | | 137 | |
| RSV105 | Heavy chain variable | A1e9 | US20140044719 SEQ ID NO: | 24685 |
| | region, F Protein | | 144 | |
| RSV106 | Heavy chain variable | A12a6 | US20140044719 SEQ ID NO: | 24686 |
| | region, F Protein | | 149 | |
| RSV107 | Heavy chain variable | A13c4 | US20140044719 SEQ ID NO: | 24687 |
| | region, F Protein | | 155 | |
| RSV108 | Heavy chain variable | A17d4 | US20140044719 SEQ ID NO: | 24688 |
| | region, F Protein | | 161 | |
| RSV109 | Heavy chain variable | A4B4 | US20140044719 SEQ ID NO: | 24689 |
| | region, F Protein | | 167 | |
| RSV110 | Heavy chain variable | A8c7 | US20140044719 SEQ ID NO: | 24690 |
| | region, F Protein | | 172 | |
| RSV111 | Heavy chain variable | IX-493L1FR | US20140044719 SEQ ID NO: | 24691 |
| | region, F Protein | | 176 | |
| RSV112 | Heavy chain variable | M3H9 | US20140044719 SEQ ID NO: | 24692 |
| | region, F Protein | | 181 | |
| RSV113 | Heavy chain variable | B21M | US20110027294 SEQ ID NO: | 24693 |
| | region, F Protein | | 49 | |
| RSV114 | Heavy chain variable | 101F | US20110027294 SEQ ID NO: 4 | 24694 |
| | region, F Protein | | | |
| RSV115 | Heavy chain variable | HNK20 | EP1720908; WO2005079479 | 24695 |
| | region, F Protein | | SEQ ID NO: 1 | |
| RSV116 | Heavy chain variable | P1212 | US20140044719 SEQ ID NO: | 24696 |
| | region, F Protein | | 123 | |
| RSV117 | Heavy chain variable | P12f4 | US20140044719 SEQ ID NO: | 24697 |
| | region, F Protein | | 132 | |
| RSV118 | Heavy chain variable | P11d4 | US20140044719 SEQ ID NO: | 24698 |
| | region, F Protein | | 138 | |
| RSV119 | Heavy chain variable | A1e9 | US20140044719 SEQ ID NO: | 24699 |
| | region, F Protein | | 145 | |
| RSV120 | Heavy chain variable | A12a6 | US20140044719 SEQ ID NO: | 24700 |
| | region, F Protein | | 150 | |
| RSV121 | Heavy chain variable | A13c4 | US20140044719 SEQ ID NO: | 24701 |
| | region, F Protein | | 156 | |
| RSV122 | Heavy chain variable | A17d4 | US20140044719 SEQ ID NO: | 24702 |
| | region, F Protein | | 162 | |
| RSV123 | Heavy chain variable | A4B4 | US20140044719 SEQ ID NO: | 24703 |
| | region, F Protein | | 168 | |
| RSV124 | Heavy chain variable | A8c7 | US20140044719 SEQ ID NO: | 24704 |
| | region, F Protein | | 173 | |
| RSV125 | Heavy chain variable | IX-493L1FR | US20140044719 SEQ ID NO: | 24705 |
| | region, F Protein | | 177 | |
| RSV126 | Heavy chain variable | H1 H3564P | WO2014159822 SEQ ID NO: 2 | 24706 |
| | region, F Protein | | | |
| RSV127 | Heavy chain variable | H1 H3565P | WO2014159822 SEQ ID NO: | 24707 |
| | region, F Protein | | 18 | |
| RSV128 | Heavy chain variable | H1 H3566P | WO2014159822 SEQ ID NO: | 24708 |
| | region, F Protein | | 34 | |
| RSV129 | Heavy chain variable | H1 H3567P | WO2014159822 SEQ ID NO: | 24709 |
| | region, F Protein | | 50 | |
| RSV130 | Heavy chain variable | H1 H3581 P | WO2014159822 SEQ ID NO: | 24710 |
| | region, F Protein | | 66 | |
| RSV131 | Heavy chain variable | H1 H3583P | WO2014159822 SEQ ID NO: | 24711 |
| | region, F Protein | | 82 | |
| RSV132 | Heavy chain variable | H1 H3589P | WO2014159822 SEQ ID NO: | 24712 |
| | region, F Protein | | 98 | |
| RSV133 | Heavy chain variable | H1 H3591 P | WO2014159822 SEQ ID NO: | 24713 |
| | region, F Protein | | 114 | |
| RSV134 | Heavy chain variable | H1 H3592P | WO2014159822 SEQ ID NO: | 24714 |
| | region, F Protein | | 130 | |
| RSV135 | Heavy chain variable | H1 H3597P | WO2014159822 SEQ ID NO: | 24715 |
| | region, F Protein | | 146 | |
| RSV136 | Heavy chain variable | H1 H3598P | WO2014159822 SEQ ID NO: | 24716 |
| | region, F Protein | | 162 | |
| RSV137 | Heavy chain variable | H1 H3603P | WO2014159822 SEQ ID NO: | 24717 |
| | region, F Protein | | 178 | |
| RSV138 | Heavy chain variable | H1 H3604P | WO2014159822 SEQ ID NO: | 24718 |
| | region, F Protein | | 194 | |
| RSV139 | Heavy chain variable | H1 H3605P | WO2014159822 SEQ ID NO: | 24719 |
| | region, F Protein | | 210 | |
| RSV140 | Heavy chain variable | H1 H3607P | WO2014159822 SEQ ID NO: | 24720 |
| | region, F Protein | | 226 | |
| RSV141 | Heavy chain variable | H1 H3608P2 | WO2014159822 SEQ ID NO: | 24721 |
| | region, F Protein | | 242 | |
| RSV142 | Heavy chain variable | H1 H3592P2 | WO2014159822 SEQ ID NO: | 24722 |
| | region, F Protein | | 258 | |
| RSV143 | Heavy chain variable | H1 H3592P3 | WO2014159822 SEQ ID NO: | 24723 |
| | region, F Protein | | 274 | |
| RSV144 | Heavy chain variable | H1 M3621 N | WO2014159822 SEQ ID NO: | 24724 |
| | region, F Protein | | 290 | |
| RSV145 | Heavy chain variable | H1 M3622N | WO2014159822 SEQ ID NO: | 24725 |
| | region, F Protein | | 306 | |
| RSV146 | Heavy chain variable | H1 M2634N | WO2014159822 SEQ ID NO: | 24726 |
| | region, F Protein | | 322 | |
| RSV147 | Heavy chain variable | H1 M3627N | WO2014159822 SEQ ID NO: | 24727 |
| | region, F Protein | | 338 | |
| RSV148 | Heavy chain variable | Clone No. 735 | US20120009623 SEQ ID NO: 1 | 24728 |
| | region, F Protein | | | |
| RSV149 | Heavy chain variable | Clone No. 736 | US20120009623 SEQ ID NO: 2 | 24729 |
| | region, F Protein | | | |
| RSV150 | Heavy chain variable | Clone No. 744 | US20120009623 SEQ ID NO: 3 | 24730 |
| | region, F Protein | | | |
| RSV151 | Heavy chain variable | Clone No. 793 | US20120009623 SEQ ID NO: 4 | 24731 |
| | region, F Protein | | | |
| RSV152 | Heavy chain variable | Clone No. 795 | US20120009623 SEQ ID NO: 5 | 24732 |
| | region, F Protein | | | |
| RSV153 | Heavy chain variable | Clone No. 796 | US20120009623 SEQ ID NO: 6 | 24733 |
| | region, F Protein | | | |
| RSV154 | Heavy chain variable | Clone No. 799 | US20120009623 SEQ ID NO: 7 | 24734 |
| | region, F Protein | | | |
| RSV155 | Heavy chain variable | Clone No. 800 | US20120009623 SEQ ID NO: 8 | 24735 |
| | region, F Protein | | | |
| RSV156 | Heavy chain variable | Clone No. 801 | US20120009623 SEQ ID NO: 9 | 24736 |
| | region, F Protein | | | |
| RSV157 | Heavy chain variable | Clone No. 804 | US20120009623 SEQ ID NO: | 24737 |
| | region, F Protein | | 10 | |
| RSV158 | Heavy chain variable | Clone No. 810 | US20120009623 SEQ ID NO: | 24738 |
| | region, F Protein | | 11 | |
| RSV159 | Heavy chain variable | Clone No. 811 | US20120009623 SEQ ID NO: | 24739 |
| | region, F Protein | | 12 | |
| RSV160 | Heavy chain variable | Clone No. 812 | US20120009623 SEQ ID NO: | 24740 |
| | region, F Protein | | 13 | |
| RSV161 | Heavy chain variable | Clone No. 814 | US20120009623 SEQ ID NO: | 24741 |
| | region, F Protein | | 14 | |
| RSV162 | Heavy chain variable | Clone No. 816 | US20120009623 SEQ ID NO: | 24742 |
| | region, F Protein | | 15 | |
| RSV163 | Heavy chain variable | Clone No. 817 | US20120009623 SEQ ID NO: | 24743 |
| | region, F Protein | | 16 | |
| RSV164 | Heavy chain variable | Clone No. 818 | US20120009623 SEQ ID NO: | 24744 |
| | region, F Protein | | 17 | |
| RSV165 | Heavy chain variable | Clone No. 819 | US20120009623 SEQ ID NO: | 24745 |
| | region, F Protein | | 18 | |
| RSV166 | Heavy chain variable | Clone No. 824 | US20120009623 SEQ ID NO: | 24746 |
| | region, F Protein | | 19 | |
| RSV167 | Heavy chain variable | Clone No. 825 | US20120009623 SEQ ID NO: | 24747 |
| | region, F Protein | | 20 | |
| RSV168 | Heavy chain variable | Clone No. 827 | US20120009623 SEQ ID NO: | 24748 |
| | region, F Protein | | 21 | |
| RSV169 | Heavy chain variable | Clone No. 829 | US20120009623 SEQ ID NO: | 24749 |
| | region, F Protein | | 22 | |
| RSV170 | Heavy chain variable | Clone No. 830 | US20120009623 SEQ ID NO: | 24750 |
| | region, F Protein | | 23 | |
| RSV171 | Heavy chain variable | Clone No. 831 | US20120009623 SEQ ID NO: | 24751 |
| | region, F Protein | | 24 | |
| RSV172 | Heavy chain variable | Clone No. 835 | US20120009623 SEQ ID NO: | 24752 |
| | region, F Protein | | 25 | |
| RSV173 | Heavy chain variable | Clone No. 838 | US20120009623 SEQ ID NO: | 24753 |
| | region, F Protein | | 26 | |
| RSV174 | Heavy chain variable | Clone No. 841 | US20120009623 SEQ ID NO: | 24754 |
| | region, F Protein | | 27 | |
| RSV175 | Heavy chain variable | Clone No. 853 | US20120009623 SEQ ID NO: | 24755 |
| | region, F Protein | | 28 | |
| RSV176 | Heavy chain variable | Clone No. 855 | US20120009623 SEQ ID NO: | 24756 |
| | region, F Protein | | 29 | |
| RSV177 | Heavy chain variable | Clone No. 856 | US20120009623 SEQ ID NO: | 24757 |
| | region, F Protein | | 30 | |
| RSV178 | Heavy chain variable | Clone No. 857 | US20120009623 SEQ ID NO: | 24758 |
| | region, F Protein | | 31 | |
| RSV179 | Heavy chain variable | Clone No. 858 | US20120009623 SEQ ID NO: | 24759 |
| | region, F Protein | | 32 | |
| RSV180 | Heavy chain variable | Clone No. 859 | US20120009623 SEQ ID NO: | 24760 |
| | region, F Protein | | 33 | |
| RSV181 | Heavy chain variable | Clone No. 861 | US20120009623 SEQ ID NO: | 24761 |
| | region, F Protein | | 34 | |
| RSV182 | Heavy chain variable | Clone No. 863 | US20120009623 SEQ ID NO: | 24762 |
| | region, F Protein | | 35 | |
| RSV183 | Heavy chain variable | Clone No. 868 | US20120009623 SEQ ID NO: | 24763 |
| | region, F Protein | | 36 | |
| RSV184 | Heavy chain variable | Clone No. 870 | US20120009623 SEQ ID NO: | 24764 |
| | region, F Protein | | 37 | |
| RSV185 | Heavy chain variable | Clone No. 871 | US20120009623 SEQ ID NO: | 24765 |
| | region, F Protein | | 38 | |
| RSV186 | Heavy chain variable | Clone No. 880 | US20120009623 SEQ ID NO: | 24766 |
| | region, F Protein | | 39 | |
| RSV187 | Heavy chain variable | Clone No. 881 | US20120009623 SEQ ID NO: | 24767 |
| | region, F Protein | | 40 | |
| RSV188 | Heavy chain variable | Clone No. 884 | US20120009623 SEQ ID NO: | 24768 |
| | region, F Protein | | 41 | |
| RSV189 | Heavy chain variable | Clone No. 886 | US20120009623 SEQ ID NO: | 24769 |
| | region, F Protein | | 42 | |
| RSV190 | Heavy chain variable | Clone No. 888 | US20120009623 SEQ ID NO: | 24770 |
| | region, F Protein | | 43 | |
| RSV191 | Heavy chain variable | Clone No. 894 | US20120009623 SEQ ID NO: | 24771 |
| | region, F Protein | | 44 | |
| RSV192 | Heavy chain variable | Gλ-1 | US20050175986 SEQ ID NO: 4 | 24772 |
| | region, F Protein | | | |
| RSV193 | Super humanized heavy | SHVh1 | EP1720908; WO2005079479 | 24773 |
| | chain based on HNK20, F | | SEQ ID NO: 3 | |
| | protein | | | |
| RSV194 | Super humanized heavy | SHVh2 | EP1720908; WO2005079479 | 24774 |
| | chain based on HNK20, F | | SEQ ID NO: 4 | |
| | protein | | | |
| RSV195 | Super humanized heavy | SHVh3 | EP1720908; WO2005079479 | 24775 |
| | chain based on HNK20, F | | SEQ ID NO: 5 | |
| | protein | | | |
| RSV196 | Super humanized heavy | SHVh4 | EP1720908; WO2005079479 | 24776 |
| | chain based on HNK20, F | | SEQ ID NO: 6 | |
| | protein | | | |
| RSV197 | Super humanized heavy | SHVh5 | EP1720908; WO2005079479 | 24777 |
| | chain based on HNK20, F | | SEQ ID NO: 7 | |
| | protein | | | |
| RSV198 | Super humanized heavy | SHVh6 | EP1720908; WO2005079479 | 24778 |
| | chain based on HNK20, F | | SEQ ID NO: 8 | |
| | protein | | | |
| RSV199 | Super humanized heavy | SHVh7 | EP1720908; WO2005079479 | 24779 |
| | chain based on HNK20, F | | SEQ ID NO: 9 | |
| | protein | | | |
| RSV200 | Heavy chain variable | B4 | EP636182; WO1993020210; | 24780 |
| | region, F Protein | | SEQ ID NO: 3 | |
| RSV201 | Heavy chain variable | B13/14 | EP636182; WO1993020210; | 24781 |
| | region, F Protein | | SEQ ID NO: 4 | |
| RSV202 | Heavy chain variable | RF-1 | EP854730; WO1996040252; | 24782 |
| | region, F Protein | | FIG. 7B | |
| RSV203 | Heavy chain variable | RF-2 | EP854730; WO1996040252; | 24783 |
| | region, F Protein | | FIG. 8B | |
| RSV204 | Heavy chain, G Protein | IF12 | U.S. Pat. No. 8,273,354 SEQ ID NO: 28 | 24784 |
| RSV205 | Heavy chain, G Protein | 3G12 | U.S. Pat. No. 8,273,354 SEQ ID NO: 29 | 24785 |
| RSV206 | Heavy chain, G Protein | 1A5 | U.S. Pat. No. 8,273,354 SEQ ID NO: 30 | 24786 |
| RSV207 | Heavy chain, G Protein | 3D3 | U.S. Pat. No. 8,273,354 SEQ ID NO: 31 | 24787 |
| RSV208 | Heavy chain, G Protein | 1G1 | U.S. Pat. No. 8,273,354 SEQ ID NO: 32 | 24788 |
| RSV209 | Heavy chain, G Protein | 2B11 | U.S. Pat. No. 8,273,354 SEQ ID NO: 33 | 24789 |
| RSV210 | Heavy chain, G Protein | 5D8 | U.S. Pat. No. 8,273,354 SEQ ID NO: 34 | 24790 |
| RSV211 | Heavy chain, G Protein | 2D10 | U.S. Pat. No. 8,273,354 SEQ ID NO: 35 | 24791 |
| RSV212 | Heavy chain, G Protein | 3F9 | U.S. Pat. No. 8,273,354 SEQ ID NO: 36 | 24792 |
| RSV213 | Heavy chain, G Protein | 1D4 | U.S. Pat. No. 8,273,354 SEQ ID NO: 37 | 24793 |
| RSV214 | Heavy chain, G Protein | 1G8 | U.S. Pat. No. 8,273,354 SEQ ID NO: 38 | 24794 |
| RSV215 | Heavy chain, G Protein | 6A12 | U.S. Pat. No. 8,273,354 SEQ ID NO: 39 | 24795 |
| RSV216 | Heavy chain, G Protein | 10C6 | U.S. Pat. No. 8,273,354 SEQ ID NO: 40 | 24796 |
| RSV217 | Heavy chain, G Protein | Hu 131-2G | U.S. Pat. No. 8,273,354 SEQ ID NO: 41 | 24797 |
| RSV218 | Heavy chain, G Protein | AT46 | US20150004155 SEQ ID NO: | 24798 |
| | | | 109 | |
| RSV219 | Heavy chain, G Protein | AT32 | US20150004155 SEQ ID NO: | 24799 |
| | | | 110 | |
| RSV220 | Heavy chain, G Protein | AT33 | US20150004155 SEQ ID NO: | 24800 |
| | | | 111 | |
| RSV221 | Heavy chain, G Protein | AT34 | US20150004155 SEQ ID NO: | 24801 |
| | | | 112 | |
| RSV222 | Heavy chain, G Protein | AT35 | US20150004155 SEQ ID NO: | 24802 |
| | | | 113 | |
| RSV223 | Heavy chain, G Protein | AT36 | US20150004155 SEQ ID NO: | 24803 |
| | | | 114 | |
| RSV224 | Heavy chain, G Protein | AT37 | US20150004155 SEQ ID NO: | 24804 |
| | | | 115 | |
| RSV225 | Heavy chain, G Protein | AT39 | US20150004155 SEQ ID NO: | 24805 |
| | | | 116 | |
| RSV226 | Heavy chain, G Protein | AT40 | US20150004155 SEQ ID NO: | 24806 |
| | | | 117 | |
| RSV227 | Heavy chain, G Protein | AT42 | US20150004155 SEQ ID NO: | 24807 |
| | | | 118 | |
| RSV228 | Heavy chain, G Protein | AT43 | US20150004155 SEQ ID NO: | 24808 |
| | | | 119 | |
| RSV229 | Heavy chain, G Protein | AT44 | US20150004155 SEQ ID NO: | 24809 |
| | | | 120 | |
| RSV230 | Heavy chain, G Protein | AT45 | US20150004155 SEQ ID NO: | 24810 |
| | | | 121 | |
| RSV231 | Heavy chain, G Protein | AT47 | US20150004155 SEQ ID NO: | 24811 |
| | | | 122 | |
| RSV232 | Heavy chain, G Protein | AT49 | US20150004155 SEQ ID NO: | 24812 |
| | | | 123 | |
| RSV233 | Heavy chain, G Protein | AT50 | US20150004155 SEQ ID NO: | 24813 |
| | | | 124 | |
| RSV234 | Heavy chain, G Protein | AT51 | US20150004155 SEQ ID NO: | 24814 |
| | | | 125 | |
| RSV235 | Heavy chain variable | CB058.1 | WO2014170257 SEQ ID NO: | 24815 |
| | region, G Protein | | 37 | |
| RSV236 | Heavy chain variable | CB048.3 | WO2014170257 SEQ ID NO: | 24816 |
| | region, G Protein | | 39 | |
| RSV237 | Heavy chain variable | CB010.7 | WO2014170257 SEQ ID NO: | 24817 |
| | region, G Protein | | 41 | |
| RSV238 | Heavy chain variable | CB003.1 | WO2014170257 SEQ ID NO: | 24818 |
| | region, G Protein | | 43 | |
| RSV239 | Heavy chain variable | CB028.2 | WO2014170257 SEQ ID NO: | 24819 |
| | region, G Protein | | 45 | |
| RSV240 | Heavy chain variable | CB002.1 | WO2014170257 SEQ ID NO: | 24820 |
| | region, G Protein | | 47 | |
| RSV241 | Heavy chain variable | CB017.3L | WO2014170258 SEQ ID NO: | 24821 |
| | region, G Protein | | 73 | |
| RSV242 | Heavy chain variable | CB017.5L | WO2014170258 SEQ ID NO: | 24822 |
| | region, G Protein | | 75 | |
| RSV243 | Heavy chain variable | CB028.1 | WO2014170258 SEQ ID NO: | 24823 |
| | region, G Protein | | 77 | |
| RSV244 | Heavy chain variable | CB030.1 | WO2014170258 SEQ ID NO: | 24824 |
| | region, G Protein | | 79 | |
| RSV245 | Heavy chain variable | CB047.1 | WO2014170258 SEQ ID NO: | 24825 |
| | region, G Protein | | 81 | |
| RSV246 | Heavy chain variable | CB047.2 | WO2014170258 SEQ ID NO: | 24826 |
| | region, G Protein | | 83 | |
| RSV247 | Heavy chain variable | CB065.1 | WO2014170258 SEQ ID NO: | 24827 |
| | region, G Protein | | 85 | |
| RSV248 | Heavy chain variable | CB071.1L | WO2014170258 SEQ ID NO: | 24828 |
| | region, G Protein | | 87 | |
| RSV249 | Heavy chain variable | CB072.1L | WO2014170258 SEQ ID NO: | 24829 |
| | region, G Protein | | 89 | |
| RSV250 | Heavy chain variable | CB073.1L | WO2014170258 SEQ ID NO: | 24830 |
| | region, G Protein | | 91 | |
| RSV251 | Heavy chain variable | CB076.2L | WO2014170258 SEQ ID NO: | 24831 |
| | region, G Protein | | 93 | |
| RSV252 | Heavy chain variable | CB079.1 | WO2014170258 SEQ ID NO: | 24832 |
| | region, G Protein | | 95 | |
| RSV253 | Heavy chain | AM14 | US20140377279 SEQ ID NO: | 24833 |
| | | | 78 | |
| RSV254 | Heavy chain | AM16 | US20140377279 SEQ ID NO: | 24834 |
| | | | 85 | |
| RSV255 | Heavy chain | AM23 | US20140377279 SEQ ID NO: | 24835 |
| | | | 92 | |
| RSV256 | Heavy chain | D25 | US20140377279 SEQ ID NO: 7 | 24836 |
| RSV257 | Heavy chain | AFFF | U.S. Pat. No. 7,635,568 SEQ ID NO: 210 | 24837 |
| RSV258 | Heavy chain | P12f2 | U.S. Pat. No. 7,635,568 SEQ ID NO: 212 | 24838 |
| RSV259 | Heavy chain | P12f4 | U.S. Pat. No. 7,635,568 SEQ ID NO: 214 | 24839 |
| RSV260 | Heavy chain | P11d4 | U.S. Pat. No. 7,635,568 SEQ ID NO: 216 | 24840 |
| RSV261 | Heavy chain | A1e9 | U.S. Pat. No. 7,635,568 SEQ ID NO: 218 | 24841 |
| RSV262 | Heavy chain | A12a6 | U.S. Pat. No. 7,635,568 SEQ ID NO: 220 | 24842 |
| RSV263 | Heavy chain | A13c4 | U.S. Pat. No. 7,635,568 SEQ ID NO: 222 | 24843 |
| RSV264 | Heavy chain | A17d4 | U.S. Pat. No. 7,635,568 SEQ ID NO: 224 | 24844 |
| RSV265 | Heavy chain | A4B4 | U.S. Pat. No. 7,635,568 SEQ ID NO: 226 | 24845 |
| RSV266 | Heavy chain | A8c7 | U.S. Pat. No. 7,635,568 SEQ ID NO: 228 | 24846 |
| RSV267 | Heavy chain | 1X-493L1FR | U.S. Pat. No. 7,635,568 SEQ ID NO: 230 | 24847 |
| RSV268 | Heavy chain | H3-3F4 | U.S. Pat. No. 7,635,568 SEQ ID NO: 232 | 24848 |
| RSV269 | Heavy chain | M3H9 | U.S. Pat. No. 7,635,568 SEQ ID NO: 234 | 24849 |
| RSV270 | Heavy chain | Y10H6 | U.S. Pat. No. 7,635,568 SEQ ID NO: 236 | 24850 |
| RSV271 | Heavy chain | DG | U.S. Pat. No. 7,635,568 SEQ ID NO: 238 | 24851 |
| RSV272 | Heavy chain | AFFF(1) | U.S. Pat. No. 7,635,568 SEQ ID NO: 240 | 24852 |
| RSV273 | Heavy chain | 6H8 | U.S. Pat. No. 7,635,568 SEQ ID NO: 242 | 24853 |
| RSV274 | Heavy chain | L1-7E5 | U.S. Pat. No. 7,635,568 SEQ ID NO: 244 | 24854 |
| RSV275 | Heavy chain | L2-15B10 | U.S. Pat. No. 7,635,568 SEQ ID NO: 246 | 24855 |
| RSV276 | Heavy chain | A13al1 | U.S. Pat. No. 7,635,568 SEQ ID NO: 248 | 24856 |
| RSV277 | Heavy chain | A1h5 | U.S. Pat. No. 7,635,568 SEQ ID NO: 250 | 24857 |
| RSV278 | Heavy chain | A4B4(1) | U.S. Pat. No. 7,635,568 SEQ ID NO: 252 | 24858 |
| RSV279 | Heavy chain | A4B4LIFR-S28R | U.S. Pat. No. 7,635,568 SEQ ID NO: 254 | 24859 |
| | | (MEDI-524, | | |
| | | Motavizumab, | | |
| | | Numax) | | |
| RSV280 | Heavy chain | A4B4-F52S | U.S. Pat. No. 7,635,568 SEQ ID NO: 256 | 24860 |
| RSV281 | Heavy chain | | U.S. Pat. No. 7,364,737 SEQ ID NO: 1 | 24861 |
| RSV282 | Heavy chain | | U.S. Pat. No. 7,364,737 SEQ ID NO: 2 | 24862 |
| RSV283 | Heavy chain variable | J variant | WO2015108967 SEQ ID NO: | 24863 |
| | region | | 12 | |
| RSV284 | Heavy chain variable | L variant | WO2015108967 SEQ ID NO: | 24864 |
| | region | | 13 | |
| RSV285 | Heavy chain variable | LA variant | WO2015108967 SEQ ID NO: | 24865 |
| | region | | 14 | |
| RSV286 | Heavy chain variable | 1G7 | WO2015108967 SEQ ID NO: | 24866 |
| | region | | 15 | |
| RSV287 | Heavy chain variable | IF5 | WO2015108967 SEQ ID NO: | 24867 |
| | region | | 16 | |
| RSV288 | Heavy chain variable | 2D10 | WO2015108967 SEQ ID NO: | 24868 |
| | region | | 17 | |
| RSV289 | Heavy chain variable | 1G7-GLM | WO2015108967 SEQ ID NO: | 24869 |
| | region | | 18 | |
| RSV290 | Heavy chain variable | B12-1 | WO2015108967 SEQ ID NO: | 24870 |
| | region | | 19 | |
| RSV291 | Heavy chain variable | E3-5 | WO2015108967 SEQ ID NO: | 24871 |
| | region | | 20 | |
| RSV292 | Heavy chain variable | E9-2 | WO2015108967 SEQ ID NO: | 24872 |
| | region | | 21 | |
| RSV293 | Heavy chain variable | 1X-493L1FR | U.S. Pat. No. 7,635,568 SEQ ID NO: 7 | 24873 |
| | region | | | |
| RSV294 | Heavy chain variable | AFFF, AFFF(1) | U.S. Pat. No. 7,635,568 SEQ ID NO: 9 | 24874 |
| | region | | | |
| RSV295 | Heavy chain variable | P12f2 | U.S. Pat. No. 7,635,568 SEQ ID NO: 17 | 24875 |
| | region | | | |
| RSV296 | Heavy chain variable | P12f4 | U.S. Pat. No. 7,635,568 SEQ ID NO: 24 | 24876 |
| | region | | | |
| RSV297 | Heavy chain variable | P11d4 | U.S. Pat. No. 7,635,568 SEQ ID NO: 28 | 24877 |
| | region | | | |
| RSV298 | Heavy chain variable | A1e9, A1h5 | U.S. Pat. No. 7,635,568 SEQ ID NO: 33 | 24878 |
| | region | | | |
| RSV299 | Heavy chain variable | A12a6 | U.S. Pat. No. 7,635,568 SEQ ID NO: 36 | 24879 |
| | region | | | |
| RSV300 | Heavy chain variable | A13c4 | U.S. Pat. No. 7,635,568 SEQ ID NO: 40 | 24880 |
| | region | | | |
| RSV301 | Heavy chain variable | A17d4 | U.S. Pat. No. 7,635,568 SEQ ID NO: 44 | 24881 |
| | region | | | |
| RSV302 | Heavy chain variable | A4B4, A4B4(1), | U.S. Pat. No. 7,635,568 SEQ ID NO: 48 | 24882 |
| | region | A4B4L1FR-S28R | | |
| | | (MEDI-524, | | |
| | | Motavizumab, | | |
| | | Numax), A4B4- | | |
| | | F52S | | |
| RSV303 | Heavy chain variable | A8c7 | U.S. Pat. No. 7,635,568 SEQ ID NO: 51 | 24883 |
| | region | | | |
| RSV304 | Heavy chain variable | H3-3F4, M3H9, | U.S. Pat. No. 7,635,568 SEQ ID NO: 55 | 24884 |
| | region | Y10H6 | | |
| RSV305 | Heavy chain variable | DG, 6H8, L1-7E5, | U.S. Pat. No. 7,635,568 SEQ ID NO: 78 | 24885 |
| | region | L2-15B10 | | |
| RSV306 | Heavy chain variable | A13al1 | U.S. Pat. No. 7,635,568 SEQ ID NO: 67 | 24886 |
| | region | | | |
| RSV307 | Heavy chain variable | | U.S. Pat. No. 7,364,742 SEQ ID NO: 7 | 24887 |
| | region | | | |
| RSV308 | Heavy chain variable | | U.S. Pat. No. 7,364,742 SEQ ID NO: 8 | 24888 |
| | region | | | |
| RSV309 | Heavy chain variable | D2E7 | EP1807111; WO2006041970 | 24889 |
| | region | | SEQ ID NO: 2 | |
| RSV310 | Heavy chain variable | 2SD4 | EP1807111; WO2006041970 | 24890 |
| | region | | SEQ ID NO: 10 | |
| RSV311 | Heavy chain, human | | Wen, X., “Structure of the | 24891 |
| | metapneumovirus fusion | | human metapneumovirus fusion | |
| | protein with | | protein with neutralizing | |
| | neutralizing antibody | | antibody identifies a | |
| | identifies a pneumovirus | | pneumovirus antigenic site”, | |
| | antigenic site, | | Nat. Struct. Mol. Biol. 19 (4), | |
| | | | 461-463 (2012), NCBI | |
| | | | Accession # 4DAG_H(220 aa) | |
| RSV312 | Heavy chain variable, M2 | 8A4/G9 - IgG | US20140348858 SEQ ID NO: 3 | 24892 |
| | 1 antigen | | | |
| RSV313 | Heavy chain, Pre fusion | HMB2435 | WO2015010792 SEQ ID NO: | 24893 |
| | RSV F protein | | 13 | |
| RSV314 | Heavy chain, Pre fusion | HMB2437 | WO2015010792 SEQ ID NO: | 24894 |
| | RSV F protein | | 29 | |
| RSV315 | Heavy chain, Pre fusion | HMB2416 | WO2015010792 SEQ ID NO: | 24895 |
| | RSV F protein | | 45 | |
| RSV316 | Heavy chain, Pre fusion | HMB2437 | WO2015010792 SEQ ID NO: | 24896 |
| | RSV F protein | | 85 | |
| RSV317 | Heavy chain, Pre fusion | CR9501 | WO2014202570 SEQ ID NO: | 24897 |
| | RSV F protein | | 53 | |
| RSV318 | Heavy chain, Pre fusion | CR9502 | WO2014202570 SEQ ID NO: | 24898 |
| | RSV F protein | | 57 | |
| RSV319 | Heavy chain 1, Pre fusion | HMB2432 | WO2015010792 SEQ ID NO: | 24899 |
| | RSV F protein | | 61 | |
| RSV320 | Heavy chain 2, Pre fusion | HMB2432 | WO2015010792 SEQ ID NO: | 24900 |
| | RSV F protein | | 65 | |
| RSV321 | Heavy chain FR LG, Pre | HMB2435 | WO2015010792 SEQ ID NO: | 24901 |
| | fusion RSV F protein | | 75 | |
| RSV322 | light chain, F and G | clone 735 | US20110189171; U.S. Pat. No. 7,879,329 | 24902 |
| | Proteins | | SEQ ID NO: 89 | |
| RSV323 | light chain, F and G | clone 736 | US20110189171; U.S. Pat. No. 7,879,329 | 24903 |
| | Proteins | | SEQ ID NO: 90 | |
| RSV324 | light chain, F and G | clone 744 | US20110189171; U.S. Pat. No. 7,879,329 | 24904 |
| | Proteins | | SEQ ID NO: 91 | |
| RSV325 | light chain, F and G | clone 793 | US20110189171; U.S. Pat. No. 7,879,329 | 24905 |
| | Proteins | | SEQ ID NO: 92 | |
| RSV326 | light chain, F and G | clone 795 | US20110189171; U.S. Pat. No. 7,879,329 | 24906 |
| | Proteins | | SEQ ID NO: 93 | |
| RSV327 | light chain, F and G | clone 796 | US20110189171; U.S. Pat. No. 7,879,329 | 24907 |
| | Proteins | | SEQ ID NO: 94 | |
| RSV328 | light chain, F and G | clone 799 | US20110189171; U.S. Pat. No. 7,879,329 | 24908 |
| | Proteins | | SEQ ID NO: 95 | |
| RSV329 | light chain, F and G | clone 800 | US20110189171; U.S. Pat. No. 7,879,329 | 24909 |
| | Proteins | | SEQ ID NO: 96 | |
| RSV330 | light chain, F and G | clone 801 | US20110189171; U.S. Pat. No. 7,879,329 | 24910 |
| | Proteins | | SEQ ID NO: 97 | |
| RSV331 | light chain, F and G | clone 804 | US20110189171; U.S. Pat. No. 7,879,329 | 24911 |
| | Proteins | | SEQ ID NO: 98 | |
| RSV332 | light chain, F and G | clone 810 | US20110189171; U.S. Pat. No. 7,879,329 | 24912 |
| | Proteins | | SEQ ID NO: 99 | |
| RSV333 | light chain, F and G | clone 811 | US20110189171; U.S. Pat. No. 7,879,329 | 24913 |
| | Proteins | | SEQ ID NO: 100 | |
| RSV334 | light chain, F and G | clone 812 | US20110189171; U.S. Pat. No. 7,879,329 | 24914 |
| | Proteins | | SEQ ID NO: 101 | |
| RSV335 | light chain, F and G | clone 814 | US20110189171; U.S. Pat. No. 7,879,329 | 24915 |
| | Proteins | | SEQ ID NO: 102 | |
| RSV336 | light chain, F and G | clone 816 | US20110189171; U.S. Pat. No. 7,879,329 | 24916 |
| | Proteins | | SEQ ID NO: 103 | |
| RSV337 | light chain, F and G | clone 817 | US20110189171; U.S. Pat. No. 7,879,329 | 24917 |
| | Proteins | | SEQ ID NO: 104 | |
| RSV338 | light chain, F and G | clone 818 | US20110189171; U.S. Pat. No. 7,879,329 | 24918 |
| | Proteins | | SEQ ID NO: 105 | |
| RSV339 | light chain, F and G | clone 819 | US20110189171; U.S. Pat. No. 7,879,329 | 24919 |
| | Proteins | | SEQ ID NO: 106 | |
| RSV340 | light chain, F and G | clone 824 | US20110189171; U.S. Pat. No. 7,879,329 | 24920 |
| | Proteins | | SEQ ID NO: 107 | |
| RSV341 | light chain, F and G | clone 825 | US20110189171; U.S. Pat. No. 7,879,329 | 24921 |
| | Proteins | | SEQ ID NO: 108 | |
| RSV342 | light chain, F and G | clone 827 | US20110189171; U.S. Pat. No. 7,879,329 | 24922 |
| | Proteins | | SEQ ID NO: 109 | |
| RSV343 | light chain, F and G | clone 829 | US20110189171; U.S. Pat. No. 7,879,329 | 24923 |
| | Proteins | | SEQ ID NO: 110 | |
| RSV344 | light chain, F and G | clone 830 | US20110189171; U.S. Pat. No. 7,879,329 | 24924 |
| | Proteins | | SEQ ID NO: 111 | |
| RSV345 | light chain, F and G | clone 831 | US20110189171; U.S. Pat. No. 7,879,329 | 24925 |
| | Proteins | | SEQ ID NO: 112 | |
| RSV346 | light chain, F and G | clone 835 | US20110189171; U.S. Pat. No. 7,879,329 | 24926 |
| | Proteins | | SEQ ID NO: 113 | |
| RSV347 | light chain, F and G | clone 838 | US20110189171; U.S. Pat. No. 7,879,329 | 24927 |
| | Proteins | | SEQ ID NO: 114 | |
| RSV348 | light chain, F and G | clone 841 | US20110189171; U.S. Pat. No. 7,879,329 | 24928 |
| | Proteins | | SEQ ID NO: 115 | |
| RSV349 | light chain, F and G | clone 853 | US20110189171; U.S. Pat. No. 7,879,329 | 24929 |
| | Proteins | | SEQ ID NO: 116 | |
| RSV350 | light chain, F and G | clone 855 | US20110189171; U.S. Pat. No. 7,879,329 | 24930 |
| | Proteins | | SEQ ID NO: 117 | |
| RSV351 | light chain, F and G | clone 856 | US20110189171; U.S. Pat. No. 7,879,329 | 24931 |
| | Proteins | | SEQ ID NO: 118 | |
| RSV352 | light chain, F and G | clone 857 | US20110189171; U.S. Pat. No. 7,879,329 | 24932 |
| | Proteins | | SEQ ID NO: 119 | |
| RSV353 | light chain, F and G | clone 858 | US20110189171; U.S. Pat. No. 7,879,329 | 24933 |
| | Proteins | | SEQ ID NO: 120 | |
| RSV354 | light chain, F and G | clone 859 | US20110189171; U.S. Pat. No. 7,879,329 | 24934 |
| | Proteins | | SEQ ID NO: 121 | |
| RSV355 | light chain, F and G | clone 861 | US20110189171; U.S. Pat. No. 7,879,329 | 24935 |
| | Proteins | | SEQ ID NO: 122 | |
| RSV356 | light chain, F and G | clone 863 | US20110189171; U.S. Pat. No. 7,879,329 | 24936 |
| | Proteins | | SEQ ID NO: 123 | |
| RSV357 | light chain, F and G | clone 868 | US20110189171; U.S. Pat. No. 7,879,329 | 24937 |
| | Proteins | | SEQ ID NO: 124 | |
| RSV358 | light chain, F and G | clone 870 | US20110189171; U.S. Pat. No. 7,879,329 | 24938 |
| | Proteins | | SEQ ID NO: 125 | |
| RSV359 | light chain, F and G | clone 871 | US20110189171; U.S. Pat. No. 7,879,329 | 24939 |
| | Proteins | | SEQ ID NO: 126 | |
| RSV360 | light chain, F and G | clone 880 | US20110189171; U.S. Pat. No. 7,879,329 | 24940 |
| | Proteins | | SEQ ID NO: 127 | |
| RSV361 | light chain, F and G | clone 881 | US20110189171; U.S. Pat. No. 7,879,329 | 24941 |
| | Proteins | | SEQ ID NO: 128 | |
| RSV362 | light chain, F and G | clone 884 | US20110189171; U.S. Pat. No. 7,879,329 | 24942 |
| | Proteins | | SEQ ID NO: 129 | |
| RSV363 | light chain, F and G | clone 886 | US20110189171; U.S. Pat. No. 7,879,329 | 24943 |
| | Proteins | | SEQ ID NO: 130 | |
| RSV364 | light chain, F and G | clone 888 | US20110189171; U.S. Pat. No. 7,879,329 | 24944 |
| | Proteins | | SEQ ID NO: 131 | |
| RSV365 | light chain, F and G | clone 894 | US20110189171; U.S. Pat. No. 7,879,329 | 24945 |
| | Proteins | | SEQ ID NO: 132 | |
| RSV366 | Light chain variable, F | 3210 variant 1, | WO2013140247 SEQ ID NO: | 24946 |
| | protein of RSV, MPV, or | 3210 variant 2, | 14 | |
| | PVM | 3210 variant 5 | | |
| RSV367 | Light chain variable, F | 2430 variant 1, | WO2013140247 SEQ ID NO: | 24947 |
| | protein of RSV, MPV, or | 2430 variant 2, | 30 | |
| | PVM | 2430 variant 4 | | |
| RSV368 | Light chain variable, F | 3210 variant 3 | WO2013140247 SEQ ID NO: | 24948 |
| | protein of RSV, MPV, or | | 37 | |
| | PVM | | | |
| RSV369 | Light chain variable, F | 3210 variant 4, | WO2013140247 SEQ ID NO: | 24949 |
| | protein of RSV, MPV, or | 3210 variant 6 | 50 | |
| | PVM | | | |
| RSV370 | Light chain variable, F | 2430 variant 3, | WO2013140247 SEQ ID NO: | 24950 |
| | protein of RSV, MPV, or | 2430 variant 5 | 60 | |
| | PVM | | | |
| RSV371 | Light chain, F Protein | clone 19 | EP1259547; U.S. Pat. No. 8,153,133 | 24951 |
| | | | SEQ ID NO: 40 | |
| RSV372 | Light chain variable | | US20140093501 SEQ ID NO: | 24952 |
| | region, CDR Grafted, F | | 20 | |
| | Protein | | | |
| RSV373 | Light chain variable | | US20140093501 SEQ ID NO: | 24953 |
| | region, CDR Grafted, F | | 34 | |
| | Protein | | | |
| RSV374 | Light chain, F Protein | AM22 | U.S. Pat. No. 8,568,726 SEQ ID NO: 32 | 24954 |
| RSV375 | Light chain, F Protein | RSVF2-5 | U.S. Pat. No. 8,221,759 SEQ ID NO: 9 | 24955 |
| ID NO: 3 | | | | |
| RSV376 | Light chain, F Protein | | EP1259547; U.S. Pat. No. 8,153,133 SEQ | 24956 |
| | | | ID NO: 3 | |
| RSV377 | Light chain, F Protein | MEDI- | EP1259547; U.S. Pat. No. 8,153,133 SEQ | 24957 |
| | | 493/Pavilizumab- | ID NO: 1 | |
| | | N-VL (Brand name | | |
| | | Synagis) | | |
| RSV378 | Light chain, F Protein | | EP1259547; U.S. Pat. No. 8,153,133 SEQ | 24958 |
| | | | ID NO: 35 | |
| RSV379 | Light chain, F Protein | clone 18 | EP1259547; U.S. Pat. No. 8,153,133 SEQ | 24959 |
| | | | ID NO: 38 | |
| RSV380 | Light chain, F Protein | clone 20 | EP1259547; U.S. Pat. No. 8,153,133 SEQ | 24960 |
| | | | ID NO: 42 | |
| RSV381 | Light chain, F Protein | clone 21 | EP1259547; U.S. Pat. No. 8,153,133 SEQ | 24961 |
| | | | ID NO: 44 | |
| RSV382 | Light chain, F Protein | clone 22 | EP1259547; U.S. Pat. No. 8,153,133 SEQ | 24962 |
| | | | ID NO: 46 | |
| RSV383 | Light chain, F Protein | clone 23 | EP1259547; U.S. Pat. No. 8,153,133 SEQ | 24963 |
| | | | ID NO: 48 | |
| RSV384 | Light chain, F Protein | clone 24 | EP1259547; U.S. Pat. No. 8,153,133 SEQ | 24964 |
| | | | ID NO: 50 | |
| RSV385 | Light chain, F Protein | clone 25 | EP1259547; U.S. Pat. No. 8,153,133 SEQ | 24965 |
| | | | ID NO: 52 | |
| RSV386 | Light chain, F Protein | clone 26 | EP1259547; U.S. Pat. No. 8,153,133 SEQ | 24966 |
| | | | ID NO: 54 | |
| RSV387 | Light chain variable | huK 102 | US20140093501 SEQ ID NO: | 24967 |
| | region, F Protein | | 19 | |
| RSV388 | Light chain variable | huK102 | US20140093501 SEQ ID NO: | 24968 |
| | region, F Protein | | 33 | |
| RSV389 | Light chain variable | RSV G8 | U.S. Pat. No. 7,867,497 SEQ ID NO: 4 | 24969 |
| | region, F Protein | | | |
| RSV390 | Light chain variable | Clone 1 | US20120135006 SEQ ID NO: | 24970 |
| | region, F Protein | | 17 | |
| RSV391 | Light chain variable | Clone 2 | US20120135006 SEQ ID NO: | 24971 |
| | region, F Protein | | 19 | |
| RSV392 | Light chain variable | Clone 3 | US20120135006 SEQ ID NO: | 24972 |
| | region, F Protein | | 21 | |
| RSV393 | Light chain variable | Clone 22 | US20120135006 SEQ ID NO: | 24973 |
| | region, F Protein | | 23 | |
| RSV394 | Light chain variable | Clone 23 | US20120135006 SEQ ID NO: | 24974 |
| | region, F Protein | | 25 | |
| RSV395 | Light chain variable | RSV13-9 | WO2009088159 SEQ ID NO: 2 | 24975 |
| | region, F Protein | | | |
| RSV396 | Light chain variable | MAb1308F | US20140093501 SEQ ID NO: | 24976 |
| | region, F Protein | | 21 | |
| RSV397 | Light chain, F Protein | 58c5 | US20140044719 SEQ ID NO: 5 | 24977 |
| RSV398 | Light chain, F Protein | sc5 | US20140044719 SEQ ID NO: | 24978 |
| | | | 13 | |
| RSV399 | Light chain, F Protein | Clone No. 735 | US20120009623 SEQ ID NO: | 24979 |
| | | | 89 | |
| RSV400 | Light chain, F Protein | Clone No. 736 | US20120009623 SEQ ID NO: | 24980 |
| | | | 90 | |
| RSV401 | Light chain, F Protein | Clone No. 744 | US20120009623 SEQ ID NO: | 24981 |
| | | | 91 | |
| RSV402 | Light chain, F Protein | Clone No. 793 | US20120009623 SEQ ID NO: | 24982 |
| | | | 92 | |
| RSV403 | Light chain, F Protein | Clone No. 795 | US20120009623 SEQ ID NO: | 24983 |
| | | | 93 | |
| RSV404 | Light chain, F Protein | Clone No. 796 | US20120009623 SEQ ID NO: | 24984 |
| | | | 94 | |
| RSV405 | Light chain, F Protein | Clone No. 799 | US20120009623 SEQ ID NO: | 24985 |
| | | | 95 | |
| RSV406 | Light chain, F Protein | Clone No. 800 | US20120009623 SEQ ID NO: | 24986 |
| | | | 96 | |
| RSV407 | Light chain, F Protein | Clone No. 80 | US20120009623 SEQ ID NO: | 24987 |
| | | | 97 | |
| RSV408 | Liglit chain, F Protein | Clone No. 804 | US20120009623 SEQ ID NO: | 24988 |
| | | | 98 | |
| RSV409 | Light chain, F Protein | Clone No. 810 | US20120009623 SEQ ID NO: | 24989 |
| | | | 99 | |
| RSV410 | Light chain, F Protein | Clone No. 811 | US20120009623 SEQ ID NO: | 24990 |
| | | | 100 | |
| RSV411 | Light chain, F Protein | Clone No. 812 | US20120009623 SEQ ID NO: | 24991 |
| | | | 101 | |
| RSV412 | Light chain, F Protein | Clone No. 814 | US20120009623 SEQ ID NO: | 24992 |
| | | | 102 | |
| RSV413 | Light chain, F Protein | Clone No. 816 | US20120009623 SEQ ID NO: | 24993 |
| | | | 103 | |
| RSV414 | Light chain, F Protein | Clone No. 817 | US20120009623 SEQ ID NO: | 24994 |
| | | | 104 | |
| RSV415 | Light chain, F Protein | Clone No. 818 | US20120009623 SEQ ID NO: | 24995 |
| | | | 105 | |
| RSV416 | Light chain, F Protein | Clone No. 819 | US20120009623 SEQ ID NO: | 24996 |
| | | | 106 | |
| RSV417 | Light chain, F Protein | Clone No. 824 | US20120009623 SEQ ID NO: | 24997 |
| | | | 107 | |
| RSV418 | Light chain, F Protein | Clone No. 825 | US20120009623 SEQ ID NO: | 24998 |
| | | | 108 | |
| RSV419 | Light chain, F Protein | Clone No. 827 | US20120009623 SEQ ID NO: | 24999 |
| | | | 109 | |
| RSV420 | Light chain, F Protein | Clone No. 829 | US20120009623 SEQ ID NO: | 25000 |
| | | | 110 | |
| RSV421 | Light chain, F Protein | Clone No. 830 | US20120009623 SEQ ID NO: | 25001 |
| | | | 111 | |
| RSV422 | Light chain, F Protein | Clone No. 831 | US20120009623 SEQ ID NO: | 25002 |
| | | | 112 | |
| RSV423 | Light chain, F Protein | Clone No. 835 | US20120009623 SEQ ID NO: | 25003 |
| | | | 113 | |
| RSV424 | Light chain, F Protein | Clone No. 838 | US20120009623 SEQ ID NO: | 25004 |
| | | | 114 | |
| RSV425 | Light chain, F Protein | Clone No. 841 | US20120009623 SEQ ID NO: | 25005 |
| | | | 115 | |
| RSV426 | Light chain, F Protein | Clone No. 853 | US20120009623 SEQ ID NO: | 25006 |
| | | | 116 | |
| RSV427 | Light chain, F Protein | Clone No. 855 | US20120009623 SEQ ID NO: | 25007 |
| | | | 117 | |
| RSV428 | Light chain, F Protein | Clone No. 856 | US20120009623 SEQ ID NO: | 25008 |
| | | | 118 | |
| RSV429 | Light chain, F Protein | Clone No. 857 | US20120009623 SEQ ID NO: | 25009 |
| | | | 119 | |
| RSV430 | Light chain, F Protein | Clone No. 858 | US20120009623 SEQ ID NO: | 25010 |
| | | | 120 | |
| RSV431 | Light chain, F Protein | Clone No. 859 | US20120009623 SEQ ID NO: | 25011 |
| | | | 121 | |
| RSV432 | Light chain, F Protein | Clone No. 861 | US20120009623 SEQ ID NO: | 25012 |
| | | | 122 | |
| RSV433 | Light chain, F Protein | Clone No. 863 | US20120009623 SEQ ID NO: | 25013 |
| | | | 123 | |
| RSV434 | Light chain, F Protein | Clone No. 868 | US20120009623 SEQ ID NO: | 25014 |
| | | | 124 | |
| RSV435 | Light chain, F Protein | Clone No. 870 | US20120009623 SEQ ID NO: | 25015 |
| | | | 125 | |
| RSV436 | Light chain, F Protein | Clone No. 871 | US20120009623 SEQ ID NO: | 25016 |
| | | | 126 | |
| RSV437 | Light chain, F Protein | Clone No. 880 | US20120009623 SEQ ID NO: | 25017 |
| | | | 127 | |
| RSV438 | Light chain, F Protein | Clone No. 881 | US20120009623 SEQ ID NO: | 25018 |
| | | | 128 | |
| RSV439 | Light chain, F Protein | Clone No. 884 | US20120009623 SEQ ID NO: | 25019 |
| | | | 129 | |
| RSV440 | Light chain, F Protein | Clone No. 886 | US20120009623 SEQ ID NO: | 25020 |
| | | | 130 | |
| RSV441 | Light chain, F Protein | Clone No. 888 | US20120009623 SEQ ID NO: | 25021 |
| | | | 131 | |
| RSV442 | Light chain, F Protein | Clone No. 894 | US20120009623 SEQ ID NO: | 25022 |
| | | | 132 | |
| RSV443 | Light chain, F Protein | | US20110027294 SEQ ID NO: | 25023 |
| | | | 63 | |
| RSV444 | Light chain, F Protein | | US20110027294 SEQ ID NO: | 25024 |
| | | | 64 | |
| RSV445 | Light chain, F Protein | | US20110027294 SEQ ID NO: | 25025 |
| | | | 65 | |
| RSV446 | Light chain, F Protein | | US20110027294 SEQ ID NO: | 25026 |
| | | | 66 | |
| RSV447 | Light chain, F Protein | | US20110027294 SEQ ID NO: | 25027 |
| | | | 67 | |
| RSV448 | Light chain, F Protein | | US20110027294 SEQ ID NO: | 25028 |
| | | | 68 | |
| RSV449 | Light chain, F Protein | | US20110027294 SEQ ID NO: | 25029 |
| | | | 69 | |
| RSV450 | Light chain, F Protein | | US20110027294 SEQ ID NO: | 25030 |
| | | | 70 | |
| RSV451 | Light chain, F Protein | | US20110027294 SEQ ID NO: | 25031 |
| | | | 71 | |
| RSV452 | Light chain, F Protein | | US20110027294 SEQ ID NO: | 25032 |
| | | | 72 | |
| RSV453 | Light chain, F Protein | | US20110027294 SEQ ID NO: | 25033 |
| | | | 73 | |
| RSV454 | Light chain, F Protein | | US20110027294 SEQ ID NO: | 25034 |
| | | | 81 | |
| RSV455 | Light chain, F Protein | | US20110027294 SEQ ID NO: | 25035 |
| | | | 82 | |
| RSV456 | Light chain, F Protein | | US20110027294 SEQ ID NO: | 25036 |
| | | | 83 | |
| RSV457 | Light chain, F Protein | | US20110027294 SEQ ID NO: | 25037 |
| | | | 84 | |
| RSV458 | Light chain, F Protein | | US20110027294 SEQ ID NO: | 25038 |
| | | | 85 | |
| RSV459 | Light chain, F Protein | | US20110027294 SEQ ID NO: | 25039 |
| | | | 86 | |
| RSV460 | Light chain, F Protein | | US20110027294 SEQ ID NO: | 25040 |
| | | | 87 | |
| RSV461 | Light chain, F Protein | | US20110027294 SEQ ID NO: | 25041 |
| | | | 88 | |
| RSV462 | Light chain, F Protein | | US20110027294 SEQ ID NO: | 25042 |
| | | | 89 | |
| RSV463 | Light chain, F Protein | | US20110027294 SEQ ID NO: | 25043 |
| | | | 90 | |
| RSV464 | Light chain, F Protein | | US20110027294 SEQ ID NO: | 25044 |
| | | | 91 | |
| RSV465 | Light chain, F Protein | | US20110027294 SEQ ID NO: | 25045 |
| | | | 92 | |
| RSV466 | Light chain, F Protein | | US20110027294 SEQ ID NO: | 25046 |
| | | | 93 | |
| RSV467 | Light chain, F Protein | | US20110027294 SEQ ID NO: | 25047 |
| | | | 94 | |
| RSV468 | Light chain, F Protein | | US20110027294 SEQ ID NO: | 25048 |
| | | | 95 | |
| RSV469 | Light chain, F Protein | | US20110027294 SEQ ID NO: | 25049 |
| | | | 96 | |
| RSV470 | Light chain, F Protein | | US20110027294 SEQ ID NO: | 25050 |
| | | | 97 | |
| RSV471 | Light chain, F Protein | | US20110027294 SEQ ID NO: | 25051 |
| | | | 98 | |
| RSV472 | Light chain, F Protein | | US20110027294 SEQ ID NO: | 25052 |
| | | | 99 | |
| RSV473 | Light chain, F Protein | | US20110027294 SEQ ID NO: | 25053 |
| | | | 100 | |
| RSV474 | Light chain, F Protein | | US20110027294 SEQ ID NO: | 25054 |
| | | | 101 | |
| RSV475 | Light chain, F Protein | | US20110027294 SEQ ID NO: | 25055 |
| | | | 102 | |
| RSV476 | Light chain, F Protein | | US20110027294 SEQ ID NO: | 25056 |
| | | | 103 | |
| RSV477 | Light chain, F Protein | | US20110027294 SEQ ID NO: | 25057 |
| | | | 104 | |
| RSV478 | Light chain, F Protein | | US20110027294 SEQ ID NO: | 25058 |
| | | | 105 | |
| RSV479 | Light chain, F Protein | | US20110027294 SEQ ID NO: | 25059 |
| | | | 106 | |
| RSV480 | Light chain, F Protein | | US20110027294 SEQ ID NO: | 25060 |
| | | | 107 | |
| RSV481 | Light chain, F Protein | | US20110027294 SEQ ID NO: | 25061 |
| | | | 108 | |
| RSV482 | Light chain, F Protein | | US20110027294 SEQ ID NO: | 25062 |
| | | | 109 | |
| RSV483 | Light chain, F Protein | | US20110027294 SEQ ID NO: | 25063 |
| | | | 110 | |
| RSV484 | Light chain, F Protein | | US20110027294 SEQ ID NO: | 25064 |
| | | | 111 | |
| RSV485 | Light chain, F Protein | | US20110027294 SEQ ID NO: | 25065 |
| | | | 112 | |
| RSV486 | Light chain, F Protein | Gλ-1A | US20050175986 SEQ ID NO: 9 | 25066 |
| RSV487 | Light chain, F Protein | A construct | US20050175986 SEQ ID NO: | 25067 |
| | | | 11 | |
| RSV488 | Light chain, F Protein | B construct | US20050175986 SEQ ID NO: | 25068 |
| | | | 12 | |
| RSV489 | Light chain, F Protein | hu19A | US20050019758; | 25069 |
| | | | WO1998019704 SEQ ID NO: | |
| | | | 10 | |
| RSV490 | Light chain, F Protein | hu19B | US20050019758; | 25070 |
| | | | WO1998019704 SEQ ID NO: | |
| | | | 11 | |
| RSV491 | Light chain, F Protein | hu19C | US20050019758; | 25071 |
| | | | WO1998019704 SEQ ID NO: | |
| | | | 12 | |
| RSV492 | Light chain, F Protein | hu19D | US20050019758; | 25072 |
| | | | WO1998019704 SEQ ID NO: | |
| | | | 13 | |
| RSV493 | Light chain, F Protein | RSV19 | EP636182; WO1993020210; | 25073 |
| | | | SEQ ID NO: 12 | |
| RSV494 | Light chain, F Protein | | WO19922004381 | 25074 |
| RSV495 | Light chain variable | P1212 | US20140044719 SEQ ID NO: | 25075 |
| | region, F Protein | | 127 | |
| RSV496 | Light chain variable | P12f4 | US20140044719 SEQ ID NO: | 25076 |
| | region, F Protein | | 134 | |
| RSV497 | Light chain variable | P11d4 | US20140044719 SEQ ID NO: | 25077 |
| | region, F Protein | | 140 | |
| RSV498 | Light chain variable | A1e9 | US20140044719 SEQ ID NO: | 25078 |
| | region, F Protein | | 146 | |
| RSV499 | Light chain variable | A12a6 | US20140044719 SEQ ID NO: | 25079 |
| | region, F Protein | | 152 | |
| RSV500 | Light chain variable | A13c4 | US20140044719 SEQ ID NO: | 25080 |
| | region, F Protein | | 158 | |
| RSV501 | Light chain variable | A17d4 | US20140044719 SEQ ID NO: | 25081 |
| | region, F Protein | | 164 | |
| RSV502 | Light chain variable | A4B4 | US20140044719 SEQ ID NO: | 25082 |
| | region, F Protein | | 169 | |
| RSV503 | Light chain variable | A8c7 | US20140044719 SEQ ID NO: | 25083 |
| | region, F Protein | | 174 | |
| RSV504 | Light chain variable | IX-493L1FR | US20140044719 SEQ ID NO: | 25084 |
| | region, F Protein | | 178 | |
| RSV505 | Light chain variable | M3H9 | US20140044719 SEQ ID NO: | 25085 |
| | region, F Protein | | 180 | |
| RSV506 | Light chain variable | B21M | US20110027294 SEQ ID NO: | 25086 |
| | region, F Protein | | 51 | |
| RSV507 | Light chain variable | 101F | US20110027294 SEQ ID NO: 6 | 25087 |
| | region, F Protein | | | |
| RSV508 | Light chain variable | HNK20 | EP1720908; WO2005079479 | 25088 |
| | region, F Protein | | SEQ ID NO: 2 | |
| RSV509 | Light chain variable | P1212 | US20140044719 SEQ ID NO: | 25089 |
| | region, F Protein | | 128 | |
| RSV510 | Light chain variable | P12f4 | US20140044719 SEQ ID NO: | 25090 |
| | region, F Protein | | 135 | |
| RSV511 | Light chain variable | P11d4 | US20140044719 SEQ ID NO: | 25091 |
| | region, F Protein | | 141 | |
| RSV512 | Light chain variable | A1e9 | US20140044719 SEQ ID NO: | 25092 |
| | region, F Protein | | 147 | |
| RSV513 | Light chain variable | A12a6 | US20140044719 SEQ ID NO: | 25093 |
| | region, F Protein | | 153 | |
| RSV514 | Light chain variable | A13c4 | US20140044719 SEQ ID NO: | 25094 |
| | region, F Protein | | 159 | |
| RSV515 | Light chain variable | A17d4 | US20140044719 SEQ ID NO: | 25095 |
| | region, F Protein | | 165 | |
| RSV516 | Light chain variable | A4B4 | US20140044719 SEQ ID NO: | 25096 |
| | region, F Protein | | 170 | |
| RSV517 | Light chain variable | A8c7 | US20140044719 SEQ ID NO: | 25097 |
| | region, F Protein | | 175 | |
| RSV518 | Light chain variable | IX-493L1FR | US20140044719 SEQ ID NO: | 25098 |
| | region, F Protein | | 179 | |
| RSV519 | Light chain variable | H1 H3564P | WO2014159822 SEQ ID NO: | 25099 |
| | region, F Protein | | 10 | |
| RSV520 | Light chain variable | H1 H3565P | WO2014159822 SEQ ID NO: | 25100 |
| | region, F Protein | | 26 | |
| RSV521 | Light chain variable | H1 H3566P | WO2014159822 SEQ ID NO: | 25101 |
| | region, F Protein | | 42 | |
| RSV522 | Light chain variable | H1 H3567P | WO2014159822 SEQ ID NO: | 25102 |
| | region, F Protein | | 58 | |
| RSV523 | Light chain variable | H1 H3581 P | WO2014159822 SEQ ID NO: | 25103 |
| | region, F Protein | | 74 | |
| RSV524 | Light chain variable | H1 H3583P | WO2014159822 SEQ ID NO: | 25104 |
| | region, F Protein | | 90 | |
| RSV525 | Light chain variable | H1 H3589P | WO2014159822 SEQ ID NO: | 25105 |
| | region, F Protein | | 106 | |
| RSV526 | Light chain variable | H1 H3591 P | WO2014159822 SEQ ID NO: | 25106 |
| | region, F Protein | | 122 | |
| RSV527 | Light chain variable | H1 H3592P | WO2014159822 SEQ ID NO: | 25107 |
| | region, F Protein | | 138 | |
| RSV528 | Light chain variable | H1 H3597P | WO2014159822 SEQ ID NO: | 25108 |
| | region, F Protein | | 154 | |
| RSV529 | Light chain variable | H1 H3598P | WO2014159822 SEQ ID NO: | 25109 |
| | region, F Protein | | 170 | |
| RSV530 | Light chain variable | H1 H3603P | WO2014159822 SEQ ID NO: | 25110 |
| | region, F Protein | | 186 | |
| RSV531 | Light chain variable | H1 H3604P | WO2014159822 SEQ ID NO: | 25111 |
| | region, F Protein | | 202 | |
| RSV532 | Light chain variable | H1 H3605P | WO2014159822 SEQ ID NO: | 25112 |
| | region, F Protein | | 218 | |
| RSV533 | Light chain variable | H1 H3607P | WO2014159822 SEQ ID NO: | 25113 |
| | region, F Protein | | 234 | |
| RSV534 | Light chain variable | H1 H3608P2 | WO2014159822 SEQ ID NO: | 25114 |
| | region, F Protein | | 250 | |
| RSV535 | Light chain variable | H1 H3592P2 | WO2014159822 SEQ ID NO: | 25115 |
| | region, F Protein | | 266 | |
| RSV536 | Light chain variable | H1 H3592P3 | WO2014159822 SEQ ID NO: | 25116 |
| | region, F Protein | | 282 | |
| RSV537 | Light chain variable | H1 M3621 N | WO2014159822 SEQ ID NO: | 25117 |
| | region, F Protein | | 298 | |
| RSV538 | Light chain variable | H1 M3622N | WO2014159822 SEQ ID NO: | 25118 |
| | region, F Protein | | 314 | |
| RSV539 | Light chain variable | H1 M2634N | WO2014159822 SEQ ID NO: | 25119 |
| | region, F Protein | | 330 | |
| RSV540 | Light chain variable | H1 M3627N | WO2014159822 SEQ ID NO: | 25120 |
| | region, F Protein | | 346 | |
| RSV541 | Light chain variable | Gλ-1 | US20050175986 SEQ ID NO: 2 | 25121 |
| | region, F Protein | | | |
| RSV542 | Light chain variable | MAb1129 | US20140093501 SEQ ID NO: | 25122 |
| | region, F Protein | | 35 | |
| RSV543 | super humanized kappa | SHVl1 | EP1720908; WO2005079479 | 25123 |
| | light chain based on | | SEQ ID NO: 10 | |
| | HNK20, F protein | | | |
| RSV544 | super humanized kappa | SHVl2 | EP1720908: WO2005079479 | 25124 |
| | light chain based on | | SEQ ID NO: 11 | |
| | HNK.20, F protein | | | |
| RSV545 | super humanized kappa | SHVl3 | EP1720908; WO2005079479 | 25125 |
| | light chain based on | | SEQ ID NO: 12 | |
| | HNK20, F protein | | | |
| RSV546 | super humanized kappa | SHVl4 | EP1720908; WO2005079479 | 25126 |
| | light chain based on | | SEQ ID NO: 13 | |
| | HNK20, F protein | | | |
| RSV547 | super humanized kappa | SHVl5 | EP1720908; WO2005079479 | 25127 |
| | light chain based on | | SEQ ID NO: 14 | |
| | HNK20, F protein | | | |
| RSV548 | super humanized kappa | SHVl6 | EP1720908; WO2005079479 | 25128 |
| | light chain based on | | SEQ ID NO: 15 | |
| | HNK20, F protein | | | |
| RSV549 | Light chain variable | B4 | EP636182; WO1993020210; | 25129 |
| | region, F Protein | | SEQ ID NO: 1 | |
| RSV550 | Light chain variable | B13/14 | EP636182; WO1993020210; | 25130 |
| | region, F Protein | | SEQ ID NO: 2 | |
| RSV551 | Light chain variable | RF-1 | EP854730; WO1996040252; | 25131 |
| | region, F Protein | | FIG. 7A | |
| RSV552 | Light chain variable | RF-2 | EP854730; WO1996040252; | 25132 |
| | region, F Protein | | FIG. 8A | |
| RSV553 | Light chain variable | CB058.1 | WO2014170257 SEQ ID NO: | 25133 |
| | region Kappa, G protein | | 38 | |
| RSV554 | Light chain variable | CB048.3 | WO2014170257 SEQ ID NO: | 25134 |
| | region Kappa, G protein | | 40 | |
| RSV555 | Light chain variable | CB010.7 | WO2014170257 SEQ ID NO: | 25135 |
| | region Kappa, G protein | | 42 | |
| RSV556 | Light chain variable | CB003.1 | WO2014170257 SEQ ID NO: | 25136 |
| | region Kappa, G protein | | 44 | |
| RSV557 | Light chain variable | CB028.2 | WO2014170257 SEQ ID NO: | 25137 |
| | region Kappa, G protein | | 46 | |
| RSV558 | Light chain variable | CB002.1 | WO2014170257 SEQ ID NO: | 25138 |
| | region Kappa, G protein | | 48 | |
| RSV559 | Light chain, G Protein | 1F12 | U.S. Pat. No. 8,273,354 SEQ ID NO: 42 | 25139 |
| RSV560 | Light chain, G Protein | 3G12 | U.S. Pat. No. 8,273,354 SEQ ID NO: 43 | 25140 |
| RSV561 | Light chain, G Protein | 1A5 | U.S. Pat. No. 8,273,354 SEQ ID NO: 44 | 25141 |
| RSV562 | Light chain, G Protein | 3D3 | U.S. Pat. No. 8,273,354 SEQ ID NO: 45 | 25142 |
| RSV563 | Light chain, G Protein | 1GI | U.S. Pat. No. 8,273,354 SEQ ID NO: 46 | 25143 |
| RSV564 | Light chain, G Protein | 2B11 | U.S. Pat. No. 8,273,354 SEQ ID NO: 47 | 25144 |
| RSV565 | Light chain, G Protein | 5D8 | U.S. Pat. No. 8,273,354 SEQ ID NO: 48 | 25145 |
| RSV566 | Light chain, G Protein | 2D10 | U.S. Pat. No. 8,273,354 SEQ ID NO: 49 | 25146 |
| RSV567 | Light chain, G Protein | 3F9 | U.S. Pat. No. 8,273,354 SEQ ID NO: 50 | 25147 |
| RSV568 | Light chain, G Protein | 1D4 | U.S. Pat. No. 8,273,354 SEQ ID NO: 51 | 25148 |
| RSV569 | Light chain, G Protein | 1G8 | U.S. Pat. No. 8,273,354 SEQ ID NO: 52 | 25149 |
| RSV570 | Light chain, G Protein | 6A12 | U.S. Pat. No. 8,273,354 SEQ ID NO: 53 | 25150 |
| RSV571 | Light chain, G Protein | 10C6 | U.S. Pat. No. 8,273,354 SEQ ID NO: 54 | 25151 |
| RSV572 | Light chain, G Protein | Hu 131-2G | U.S. Pat. No. 8,273,354 SEQ ID NO: 55 | 25152 |
| RSV573 | Light chain, G Protein | AT46 | US20150004155 SEQ ID NO: | 25153 |
| | | | 127 | |
| RSV574 | Light chain, G Protein | AT32 | US20150004155 SEQ ID NO: | 25154 |
| | | | 128 | |
| RSV575 | Light chain, G Protein | AT33 | US20150004155 SEQ ID NO: | 25155 |
| | | | 129 | |
| RSV576 | Light chain, G Protein | AT34 | US20150004155 SEQ ID NO: | 25156 |
| | | | 130 | |
| RSV577 | Light chain, G Protein | AT35 | US20150004155 SEQ ID NO: | 25157 |
| | | | 131 | |
| RSV578 | Light chain, G Protein | AT36 | US20150004155 SEQ ID NO: | 25158 |
| | | | 132 | |
| RSV579 | Light chain, G Protein | AT37 | US20150004155 SEQ ID NO: | 25159 |
| | | | 133 | |
| 134 | | | | |
| RSV580 | Light chain, G Protein | AT39 | US20150004155 SEQ ID NO: | 25160 |
| | | | 134 | |
| RSV581 | Light chain, G Protein | AT40 | US20150004155 SEQ ID NO: | 25161 |
| | | | 135 | |
| RSV582 | Light chain, G Protein | AT42 | US20150004155 SEQ ID NO: | 25162 |
| | | | 136 | |
| RSV583 | Light chain, G Protein | AT43 | US20150004155 SEQ ID NO: | 25163 |
| | | | 137 | |
| RSV584 | Light chain, G Protein | AT44 | US20150004155 SEQ ID NO: | 25164 |
| | | | 138 | |
| RSV585 | Light chain, G Protein | AT45 | US20150004155 SEQ ID NO: | 25165 |
| | | | 139 | |
| RSV586 | Light chain, G Protein | AT47 | US20150004155 SEQ ID NO: | 25166 |
| | | | 140 | |
| RSV587 | Light chain, G Protein | AT49 | US20150004155 SEQ ID NO: | 25167 |
| | | | 141 | |
| RSV588 | Light chain, G Protein | AT50 | US20150004155 SEQ ID NO: | 25168 |
| | | | 142 | |
| RSV589 | Light chain, G Protein | AT51 | US20150004155 SEQ ID NO: | 25169 |
| | | | 143 | |
| RSV590 | Light chain variable | CB017.3L | WO2014170258 SEQ ID NO: | 25170 |
| | region, G Protein | | 74 | |
| RSV591 | Light chain variable | CB017.5L | WO2014170258 SEQ ID NO: | 25171 |
| | region, G Protein | | 76 | |
| RSV592 | Light chain variable | CB028.1 | WO2014170258 SEQ ID NO: | 25172 |
| | region, G Protein | | 78 | |
| RSV593 | Light chain variable | CB030.1 | WO2014170258 SEQ ID NO: | 25173 |
| | region, G Protein | | 80 | |
| RSV594 | Light chain variable | CB047.1 | WO2014170258 SEQ ID NO: | 25174 |
| | region, G Protein | | 82 | |
| RSV595 | Light chain variable | CB047.2 | WO2014170258 SEQ ID NO: | 25175 |
| | region, G Protein | | 84 | |
| RSV596 | Light chain variable | CB065.1 | WO2014170258 SEQ ID NO: | 25176 |
| | region, G Protein | | 86 | |
| RSV597 | Light chain variable | CB071.1L | WO2014170258 SEQ ID NO: | 25177 |
| | region, G Protein | | 88 | |
| RSV598 | Light chain variable | CB072.1L | WO2014170258 SEQ ID NO: | 25178 |
| | region, G Protein | | 90 | |
| RSV599 | Light chain variable | CB073.1L | WO2014170258 SEQ ID NO: | 25179 |
| | region, G Protein | | 92 | |
| RSV600 | Light chain variable | CB076.2L | WO2014170258 SEQ ID NO: | 25180 |
| | region, G Protein | | 94 | |
| RSV601 | Light chain variable | CB079.1 | WO2014170258 SEQ ID NO: | 25181 |
| | region, G Protein | | 96 | |
| RSV602 | Light chain, human | | Wen,X., “Structure of the | 25182 |
| | metapneumovirus fusion | | human metapneumovirus fusion | |
| | protein with | | protein with neutralizing | |
| | neutralizing antibody | | antibody identifies a | |
| | identifies a pneumovirus | | pneumovirus antigenic site”, | |
| | antigenic site | | Nat. Struct. Mol. Biol. 19 (4), | |
| | | | 461-463 (2012), NCBI | |
| | | | Accession # 4DAG L(213 aa) | |
| RSV603 | Light chain | AM14 | US20140377279 SEQ ID NO: | 25183 |
| | | | 79 | |
| RSV604 | Light chain | AM16 | US20140377279 SEQ ID NO: | 25184 |
| | | | 86 | |
| RSV605 | Light chain | AM23 | US20140377279 SEQ ID NO: | 25185 |
| | | | 93 | |
| RSV606 | Light chain | D25 | US20140377279 SEQ ID NO: 8 | 25186 |
| RSV607 | Light chain | AFFF | U.S. Pat. No. 7,635,568 SEQ ID NO: 211 | 25187 |
| RSV608 | Light chain | P12f2 | U.S. Pat. No. 7,635,568 SEQ ID NO: 213 | 25188 |
| RSV609 | Light chain | P12f4 | U.S. Pat. No. 7,635,568 SEQ ID NO: 215 | 25189 |
| RSV610 | Light chain | P11d4 | U.S. Pat. No. 7,635,568 SEQ ID NO: 217 | 25190 |
| RSV611 | Light chain | A1e9 | U.S. Pat. No. 7,635,568 SEQ ID NO: 219 | 25191 |
| RSV612 | Light chain | A12a6 | U.S. Pat. No. 7,635,568 SEQ ID NO: 221 | 25192 |
| RSV613 | Light chain | A13c4 | U.S. Pat. No. 7,635,568 SEQ ID NO: 223 | 25193 |
| RSV614 | Light chain | A17d4 | U.S. Pat. No. 7,635,568 SEQ ID NO: 225 | 25194 |
| RSV615 | Light chain | A4B4 | U.S. Pat. No. 7,635,568 SEQ ID NO: 227 | 25195 |
| RSV616 | Light chain | A8c7 | U.S. Pat. No. 7,635,568 SEQ ID NO: 229 | 25196 |
| RSV617 | Light chain | 1X-493L1FR | U.S. Pat. No. 7,635,568 SEQ ID NO: 231 | 25197 |
| RSV618 | Light chain | H3-3F4 | U.S. Pat. No. 7,635,568 SEQ ID NO: 233 | 25198 |
| RSV619 | Light chain | M3H9 | U.S. Pat. No. 7,635,568 SEQ ID NO: 235 | 25199 |
| RSV620 | Light chain | Y10H6 | U.S. Pat. No. 7,635,568 SEQ ID NO: 237 | 25200 |
| RSV621 | Light chain | DG | U.S. Pat. No. 7,635,568 SEQ ID NO: 239 | 25201 |
| RSV622 | Light chain | AFFF(1) | U.S. Pat. No. 7,635,568 SEQ ID NO: 241 | 25202 |
| RSV623 | Light chain | 6H8 | U.S. Pat. No. 7,635,568 SEQ ID NO: 243 | 25203 |
| RSV624 | Light chain | L1-7E5 | U.S. Pat. No. 7,635,568 SEQ ID NO: 245 | 25204 |
| RSV625 | Light chain | L2-15B10 | U.S. Pat. No. 7,635,568 SEQ ID NO: 247 | 25205 |
| RSV626 | Light chain | A13a11 | U.S. Pat. No. 7,635,568 SEQ ID NO: 249 | 25206 |
| RSV627 | Light chain | A1h5 | U.S. Pat. No. 7,635,568 SEQ ID NO: 251 | 25207 |
| RSV628 | Light chain | A4B4(1) | U.S. Pat. No. 7,635,568 SEQ ID NO: 253 | 25208 |
| RSV629 | Light chain | A4B4L1FR-S28R | U.S. Pat. No. 7,635,568 SEQ ID NO: 255 | 25209 |
| RSV630 | Light chain | A4B4-F52S | U.S. Pat. No. 7,635,568 SEQ ID NO: 257 | 25210 |
| RSV631 | Light chain variable | AFFF | U.S. Pat. No. 7,635,568 SEQ ID NO: 13 | 25211 |
| | region | | | |
| RSV632 | Light chain variable | P12f2 | U.S. Pat. No. 7,635,568 SEQ ID NO: 21 | 25212 |
| | region | | | |
| RSV633 | Light chain variable | P12f4 | U.S. Pat. No. 7,635,568 SEQ ID NO: 26 | 25213 |
| | region | | | |
| RSV634 | Light chain variable | P11d4 | U.S. Pat. No. 7,635,568 SEQ ID NO: 30 | 25214 |
| | region | | | |
| RSV635 | Light chain variable | A1e9 | U.S. Pat. No. 7,635,568 SEQ ID NO: 34 | 25215 |
| | region | | | |
| RSV636 | Light chain variable | A12a6 | U.S. Pat. No. 7,635,568 SEQ ID NO: 38 | 25216 |
| | region | | | |
| RSV637 | Light chain variable | A13c4 | U.S. Pat. No. 7,635,568 SEQ ID NO: 42 | 25217 |
| | region | | | |
| RSV638 | Light chain variable | A17d4 | U.S. Pat. No. 7,635,568 SEQ ID NO: 46 | 25218 |
| | region | | | |
| RSV639 | Light chain variable | A4B4 | U.S. Pat. No. 7,635,568 SEQ ID NO: 49 | 25219 |
| | region | | | |
| RSV640 | Light chain variable | A8c7 | U.S. Pat. No. 7,635,568 SEQ ID NO: 52 | 25220 |
| | region | | | |
| RSV641 | Light chain variable | 1X-493L1FR | U.S. Pat. No. 7,635,568 SEQ ID NO: 54 | 25221 |
| | region | | | |
| RSV642 | Light chain variable | H3-3F4, DG | U.S. Pat. No. 7,635,568 SEQ ID NO: 56 | 25222 |
| | region | | | |
| RSV643 | Light chain variable | M3H9 | U.S. Pat. No. 7,635,568 SEQ ID NO: 70 | 25223 |
| | region | | | |
| region | | | | |
| RSV644 | Light chain variable | Y10H6 | U.S. Pat. No. 7,635,568 SEQ ID NO: 58 | 25224 |
| | region | | | |
| RSV645 | Light chain variable | AFFF(1) | U.S. Pat. No. 7,635,568 SEQ ID NO: 60 | 25225 |
| | region | | | |
| RSV646 | Light chain variable | 6H8 | U.S. Pat. No. 7,635,568 SEQ ID NO: 62 | 25226 |
| | region | | | |
| RSV647 | Light chain variable | L1-7E5 | U.S. Pat. No. 7,635,568 SEQ ID NO: 64 | 25227 |
| | region | | | |
| RSV648 | Light chain variable | L2-15B10 | U.S. Pat. No. 7,635,568 SEQ ID NO: 65 | 25228 |
| | region | | | |
| RSV649 | Light chain variable | A13a11 | U.S. Pat. No. 7,635,568 SEQ ID NO: 68 | 25229 |
| | region | | | |
| RSV650 | Light chain variable | A1h5 | U.S. Pat. No. 7,635,568 SEQ ID NO: 71 | 25230 |
| | region | | | |
| RSV651 | Light chain variable | A4B4(1) | U.S. Pat. No. 7,635,568 SEQ ID NO: 74 | 25231 |
| | region | | | |
| RSV652 | Light chain variable | A4B4L1FR-S28R | U.S. Pat. No. 7,635,568 SEQ ID NO: 11 | 25232 |
| | region | | | |
| RSV653 | Light chain variable | A4B4-F52S | U.S. Pat. No. 7,635,568 SEQ ID NO: 76 | 25233 |
| | region | | | |
| RSV654 | Light chain variable | 6H; 11H; 21H; | U.S. Pat. No. 7,364,737 SEQ ID NO: 21 | 25234 |
| | region | 22H; and 23H | | |
| RSV655 | Light chain variable | 13H and 19H | U.S. Pat. No. 7,364,737 SEQ ID NO: 22 | 25235 |
| | region | | | |
| RSV656 | Light chain variable | 6L; 11L; 21L; and | U.S. Pat. No. 7,364,737 SEQ ID NO: 23 | 25236 |
| | region | 22L | | |
| RSV657 | Light chain variable | 23L | U.S. Pat. No. 7,364,737 SEQ ID NO: 24 | 25237 |
| | region | | | |
| RSV658 | Light chain variable | 13L and 19L | U.S. Pat. No. 7,364,737 SEQ ID NO: 25 | 25238 |
| | region | | | |
| RSV659 | Light chain variable | | U.S. Pat. No. 7,364,742 SEQ ID NO: 9 | 25239 |
| | region | | | |
| RSV660 | Light chain variable | | U.S. Pat. No. 7,364,742 SEQ ID NO: 10 | 25240 |
| | region | | | |
| RSV661 | Light chain variable | | U.S. Pat. No. 7,364,742 SEQ ID NO: 11 | 25241 |
| | region | | | |
| RSV662 | Light chain variable | | U.S. Pat. No. 7,364,742 SEQ ID NO: 12 | 25242 |
| | region | | | |
| RSV663 | Light chain variable | D2E7 | EP1807111; WO2006041970 | 25243 |
| | region | | SEQ ID NO: 1 | |
| RSV664 | Light chain variable | 2SD4 | EP1807111; WO2006041970 | 25244 |
| | region | | SEQ ID NO: 9 | |
| RSV665 | Light chain variable, M2 | 8A4/G9 - IgG | US20140348858 SEQ ID NO: 4 | 25245 |
| | 1 antigen | | | |
| RSV666 | Light chain, Pre fusion | HMB2435 | WO2015010792 SEQ ID NO: | 25246 |
| | RSV F protein | | 14 | |
| RSV667 | Light chain, Pre fusion | HMB2437 | WO2015010792 SEQ ID NO: | 25247 |
| | RSV F protein | | 30 | |
| RSV668 | Light chain, Pre fusion | HMB2416 | WO2015010792 SEQ ID NO: | 25248 |
| | RSV F protein | | 46 | |
| RSV669 | Light chain, Pre fusion | HMB2437 | WO2015010792 SEQ ID NO: | 25249 |
| | RSV F protein | | 86 | |
| RSV670 | Light chain, Pre fusion | CR9501 | WO2014202570 SEQ ID NO: | 25250 |
| | RSV F protein | | 61 | |
| RSV671 | Light chain, Pre fusion | CR9502 | WO2014202570 SEQ ID NO: | 25251 |
| | RSV F protein | | 65 | |
| RSV672 | Light chain 1, Pre fusion | HMB2432 | WO2015010792 SEQ ID NO: | 25252 |
| | RSV F protein | | 62 | |
| RSV673 | Light chain FR GL, Pre | HMB2432 | WO2015010792 SEQ ID NO: | 25253 |
| | fusion RSV F protein | | 66 | |
| RSV674 | Light chain 2, Pre fusion | HMB2435 | WO2015010792 SEQ ID NO: | 25254 |
| | RSV F protein | | 76 | |
| RSV675 | derived Ig variable region | RSV19VH | EP636182; WO1993020210; | 25255 |
| | amino acid sequence, F | | SEQ ID NO: 13 | |
| | protein | | | |
| RSV676 | derived Ig variable region | pHuRSV19VH | EP636182; WO1993020210; | 25256 |
| | amino acid sequence, F | | SEQ ID NO: 14 | |
| | protein | | | |
| RSV677 | derived Ig variable region | pHuRSV19VHFNS | EP636182; WO1993020210; | 25257 |
| | amino acid sequence, F | | SEQ ID NO: 15 | |
| | protein | | | |
| RSV678 | derived Ig variable region | pHuRSV19VHNIK | EP636182; WO1993020210; | 25258 |
| | amino acid sequence, F | | SEQ ID NO: 16 | |
| | protein | | | |
| RSV679 | derived Ig variable region | pHuRSV19VK | EP636182; WO1993020210; | 25259 |
| | amino acid sequence, F | | SEQ ID NO: 17 | |
| | protein | | | |
| RSV680 | Nanobody binding to | LG202A10 | US20110182897 SEQ ID NO: | 25260 |
| | RSV F protein | | 126 | |
| RSV681 | Nanobody binding to | LG202A12 | US20110182897 SEQ ID NO: | 25261 |
| | RSV F protein | | 127 | |
| RSV682 | Nanobody binding to | LG202A5 | US20110182897 SEQ ID NO: | 25262 |
| | RSV F protein | | 128 | |
| RSV683 | Nanobody binding to | LG202A9 | US20110182897 SEQ ID NO: | 25263 |
| | RSV F protein | | 129 | |
| RSV684 | Nanobody binding to | LG202B10 | US20110182897 SEQ ID NO: | 25264 |
| | RSV F protein | | 130 | |
| RSV685 | Nanobody binding to | LG202B7 | US20110182897 SEQ ID NO: | 25265 |
| | RSV F protein | | 131 | |
| RSV686 | Nanobody binding to | LG202B8 | US20110182897 SEQ ID NO: | 25266 |
| | RSV F protein | | 132 | |
| RSV687 | Nanobody binding to | LG202B9 | US20110182897 SEQ ID NO: | 25267 |
| | RSV F protein | | 133 | |
| RSV688 | Nanobody binding to | LG202C1 | US20110182897 SEQ ID NO: | 25268 |
| | RSV F protein | | 134 | |
| RSV689 | Nanobody binding to | LG202C11 | US20110182897 SEQ ID NO: | 25269 |
| | RSV F protein | | 135 | |
| RSV690 | Nanobody binding to | LG202C2 | US20110182897 SEQ ID NO: | 25270 |
| | RSV F protein | | 136 | |
| RSV691 | Nanobody binding to | LG202C7 | US20110182897 SEQ ID NO: | 25271 |
| | RSV F protein | | 137 | |
| RSV692 | Nanobody binding to | LG202C8 | US20110182897 SEQ ID NO: | 25272 |
| | RSV F protein | | 138 | |
| RSV693 | Nanobody binding to | LG202C9 | US20110182897 SEQ ID NO: | 25273 |
| | RSV F protein | | 139 | |
| RSV694 | Nanobody binding to | LG202D5 | US20110182897 SEQ ID NO: | 25274 |
| | RSV F protein | | 140 | |
| RSV695 | Nanobody binding to | LG202D7 | US20110182897 SEQ ID NO: | 25275 |
| | RSV F protein | | 141 | |
| RSV696 | Nanobody binding to | LG202D8 | US20110182897 SEQ ID NO: | 25276 |
| | RSV F protein | | 142 | |
| RSV697 | Nanobody binding to | LG202E11 | US20110182897 SEQ ID NO: | 25277 |
| | RSV F protein | | 143 | |
| RSV698 | Nanobody binding to | LG202E2 | US20110182897 SEQ ID NO: | 25278 |
| | RSV F protein | | 144 | |
| RSV699 | Nanobody binding to | LG202E5 | US20110182897 SEQ ID NO: | 25279 |
| | RSV F protein | | 145 | |
| RSV700 | Nanobody binding to | LG202E6 | US20110182897 SEQ ID NO: | 25280 |
| | RSV F protein | | 146 | |
| RSV701 | Nanobody binding to | LG202E7 | US20110182897 SEQ ID NO: | 25281 |
| | RSV F protein | | 147 | |
| RSV702 | Nanobody binding to | LG202F10 | US20110182897 SEQ ID NO: | 25282 |
| | RSV F protein | | 148 | |
| RSV703 | Nanobody binding to | LG202F12 | US20110182897 SEQ ID NO: | 25283 |
| | RSV F protein | | 149 | |
| RSV704 | Nanobody binding to | LG202F3 | US20110182897 SEQ ID NO: | 25284 |
| | RSV F protein | | 150 | |
| RSV705 | Nanobody binding to | LG202F4 | US20110182897 SEQ ID NO: | 25285 |
| | RSV F protein | | 151 | |
| RSV706 | Nanobody binding to | LG202F8 | US20110182897 SEQ ID NO: | 25286 |
| | RSV F protein | | 152 | |
| RSV707 | Nanobody binding to | LG202G11 | US20110182897 SEQ ID NO: | 25287 |
| | RSV F protein | | 153 | |
| RSV708 | Nanobody binding to | LG202G3 | US20110182897 SEQ ID NO: | 25288 |
| | RSV F protein | | 154 | |
| RSV709 | Nanobody binding to | LG202G8 | US20110182897 SEQ ID NO: | 25289 |
| | RSV F protein | | 155 | |
| RSV710 | Nanobody binding to | LG202H2 | US20110182897 SEQ ID NO: | 25290 |
| | RSV F protein | | 156 | |
| RSV711 | Nanobody binding to | LG202H8 | US20110182897 SEQ ID NO: | 25291 |
| | RSV F protein | | 157 | |
| RSV712 | Nanobody binding to | LG191B9 | US20110182897 SEQ ID NO: | 25292 |
| | RSV F protein | | 158 | |
| RSV713 | Nanobody binding to | LG191D3 | US20110182897 SEQ ID NO: | 25293 |
| | RSV F protein | | 159 | |
| RSV714 | Nanobody binding to | LG192A8 | US20110182897 SEQ ID NO: | 25294 |
| | RSV F protein | | 160 | |
| RSV715 | Nanobody binding to | LG192B1 | US20110182897 SEQ ID NO: | 25295 |
| | RSV F protein | | 161 | |
| RSV716 | Nanobody binding to | LG192C10 | US20110182897 SEQ ID NO: | 25296 |
| | RSV F protein | | 162 | |
| RSV717 | Nanobody binding to | LG192C4 | US20110182897 SEQ ID NO: | 25297 |
| | RSV F protein | | 163 | |
| RSV718 | Nanobody binding to | LG192C6 | US20110182897 SEQ ID NO: | 25298 |
| | RSV F protein | | 164 | |
| RSV719 | Nanobody binding to | LG192D3 | US20110182897 SEQ ID NO: | 25299 |
| | RSV F protein | | 165 | |
| RSV720 | Nanobody binding to | LG191E4 | US20110182897 SEQ ID NO: | 25300 |
| | RSV F protein | | 166 | |
| RSV721 | Nanobody binding to | LG192F2 | US20110182897 SEQ ID NO: | 25301 |
| | RSV F protein | | 167 | |
| RSV722 | Nanobody binding to | LG192H1 | US20110182897 SEQ ID NO: | 25302 |
| | RSV F protein | | 168 | |
| RSV723 | Nanobody binding to | LG192H2 | US20110182897 SEQ ID NO: | 25303 |
| | RSV F protein | | 169 | |
| RSV724 | Nanobody binding to | LG20610B | US20110182897 SEQ ID NO: | 25304 |
| | RSV F protein | | 170 | |
| RSV725 | Nanobody binding to | LG20610C | US20110182897 SEQ ID NO: | 25305 |
| | RSV F protein | | 171 | |
| RSV726 | Nanobody binding to | LG20610D | US20110182897 SEQ ID NO: | 25306 |
| | RSV F protein | | 172 | |
| RSV727 | Nanobody binding to | LG20610E | US20110182897 SEQ ID NO: | 25307 |
| | RSV F protein | | 173 | |
| RSV728 | Nanobody binding to | LG20610F | US20110182897 SEQ ID NO: | 25308 |
| | RSV F protein | | 174 | |
| RSV729 | Nanobody binding to | LG20611D | US20110182897 SEQ ID NO: | 25309 |
| | RSV F protein | | 175 | |
| RSV730 | Nanobody binding to | LG20611H | US20110182897 SEQ ID NO: | 25310 |
| | RSV F protein | | 176 | |
| RSV731 | Nanobody binding to | LG20612F | US20110182897 SEQ ID NO: | 25311 |
| | RSV F protein | | 177 | |
| RSV732 | Nanobody binding to | LG2062A | US20110182897 SEQ ID NO: | 25312 |
| | RSV F protein | | 178 | |
| RSV733 | Nanobody binding to | LG2062C | US20110182897 SEQ ID NO: | 25313 |
| | RSV F protein | | 179 | |
| RSV734 | Nanobody binding to | LG2062E | US20110182897 SEQ ID NO: | 25314 |
| | RSV F protein | | 180 | |
| RSV735 | Nanobody binding to | LG2062F | US20110182897 SEQ ID NO: | 25315 |
| | RSV F protein | | 181 | |
| RSV736 | Nanobody binding to | LG2062G | US20110182897 SEQ ID NO: | 25316 |
| | RSV F protein | | 182 | |
| RSV737 | Nanobody binding to | LG2062H | US20110182897 SEQ ID NO: | 25317 |
| | RSV F protein | | 183 | |
| RSV738 | Nanobody binding to | LG2063A | US20110182897 SEQ ID NO: | 25318 |
| | RSV F protein | | 184 | |
| RSV739 | Nanobody binding to | LG2063B | US20110182897 SEQ ID NO: | 25319 |
| | RSV F protein | | 185 | |
| RSV740 | Nanobody binding to | LG2063C | US20110182897 SEQ ID NO: | 25320 |
| | RSV F protein | | 186 | |
| RSV741 | Nanobody binding to | LG2063D | US20110182897 SEQ ID NO: | 25321 |
| | RSV F protein | | 187 | |
| RSV742 | Nanobody binding to | LG2063E | US20110182897 SEQ ID NO: | 25322 |
| | RSV F protein | | 188 | |
| RSV743 | Nanobody binding to | LG2063F | US20110182897 SEQ ID NO: | 25323 |
| | RSV F protein | | 189 | |
| RSV744 | Nanobody binding to | LG2064D | US20110182897 SEQ ID NO: | 25324 |
| | RSV F protein | | 190 | |
| RSV745 | Nanobody binding to | LG2064G | US20110182897 SEQ ID NO: | 25325 |
| | RSV F protein | | 191 | |
| RSV746 | Nanobody binding to | LG2065A | US20110182897 SEQ ID NO: | 25326 |
| | RSV F protein | | 192 | |
| RSV747 | Nanobody binding to | LG2065E | US20110182897 SEQ ID NO: | 25327 |
| | RSV F protein | | 193 | |
| RSV748 | Nanobody binding to | LG2066A | US20110182897 SEQ ID NO: | 25328 |
| | RSV F protein | | 194 | |
| RSV749 | Nanobody binding to | LG2066D | US20110182897 SEQ ID NO: | 25329 |
| | RSV F protein | | 195 | |
| RSV750 | Nanobody binding to | LG2067B | US20110182897 SEQ ID NO: | 25330 |
| | RSV F protein | | 196 | |
| RSV751 | Nanobody binding to | LG2067C | US20110182897 SEQ ID NO: | 25331 |
| | RSV F protein | | 197 | |
| RSV752 | Nanobody binding to | LG2067E | US20110182897 SEQ ID NO: | 25332 |
| | RSV F protein | | 198 | |
| RSV753 | Nanobody binding to | LG2067G | US20110182897 SEQ ID NO: | 25333 |
| | RSV F protein | | 199 | |
| RSV754 | Nanobody binding to | LG2067H | US20110182897 SEQ ID NO: | 25334 |
| | RSV F protein | | 200 | |
| RSV755 | Nanobody binding to | LG20711A | US20110182897 SEQ ID NO: | 25335 |
| | RSV F protein | | 201 | |
| RSV756 | Nanobody binding to | LG20711B | US20110182897 SEQ ID NO: | 25336 |
| | RSV F protein | | 202 | |
| RSV757 | Nanobody binding to | LG20711D | US20110182897 SEQ ID NO: | 25337 |
| | RSV F protein | | 203 | |
| RSV758 | Nanobody binding to | LG20711E | US20110182897 SEQ ID NO: | 25338 |
| | RSV F protein | | 204 | |
| RSV759 | Nanobody binding to | LG20711F | US20110182897 SEQ ID NO: | 25339 |
| | RSV F protein | | 205 | |
| RSV760 | Nanobody binding to | LG20711G | US20110182897 SEQ ID NO: | 25340 |
| | RSV F protein | | 206 | |
| RSV761 | Nanobody binding to | LG20711H | US20110182897 SEQ ID NO: | 25341 |
| | RSV F protein | | 207 | |
| RSV762 | Nanobody binding to | LG2071A | US20110182897 SEQ ID NO: | 25342 |
| | RSV F protein | | 208 | |
| RSV763 | Nanobody binding to | LG2071B | US20110182897 SEQ ID NO: | 25343 |
| | RSV F protein | | 209 | |
| RSV764 | Nanobody binding to | LG2071C | US20110182897 SEQ ID NO: | 25344 |
| | RSV F protein | | 210 | |
| RSV765 | Nanobody binding to | LG207D1 | US20110182897 SEQ ID NO: | 25345 |
| | RSV F protein | | 211 | |
| RSV766 | Nanobody binding to | LG2071E | US20110182897 SEQ ID NO: | 25346 |
| | RSV F protein | | 212 | |
| RSV767 | Nanobody binding to | LG2071F | US20110182897 SEQ ID NO: | 25347 |
| | RSV F protein | | 213 | |
| RSV768 | Nanobody binding to | LG2074A | US20110182897 SEQ ID NO: | 25348 |
| | RSV F protein | | 214 | |
| RSV769 | Nanobody binding to | LG2074B | US20110182897 SEQ ID NO: | 25349 |
| | RSV F protein | | 215 | |
| RSV770 | Nanobody binding to | LG2074D | US20110182897 SEQ ID NO: | 25350 |
| | RSV F protein | | 216 | |
| RSV771 | Nanobody binding to | LG2074H | US20110182897 SEQ ID NO: | 25351 |
| | RSV F protein | | 217 | |
| RSV772 | Nanobody binding to | LG2075A | US20110182897 SEQ ID NO: | 25352 |
| | RSV F protein | | 218 | |
| RSV773 | Nanobody binding to | LG2075B | US20110182897 SEQ ID NO: | 25353 |
| | RSV F protein | | 219 | |
| RSV774 | Nanobody binding to | LG2075C | US20110182897 SEQ ID NO: | 25354 |
| | RSV F protein | | 220 | |
| RSV775 | Nanobody binding to | LG2075D | US20110182897 SEQ ID NO: | 25355 |
| | RSV F protein | | 221 | |
| RSV776 | Nanobody binding to | LG2075E | US20110182897 SEQ ID NO: | 25356 |
| | RSV F protein | | 222 | |
| RSV777 | Nanobody binding to | LG2076A | US20110182897 SEQ ID NO: | 25357 |
| | RSV F protein | | 223 | |
| RSV778 | Nanobody binding to | LG2076B | US20110182897 SEQ ID NO: | 25358 |
| | RSV F protein | | 224 | |
| RSV779 | Nanobody binding to | LG2076C | US20110182897 SEQ ID NO: | 25359 |
| | RSV F protein | | 225 | |
| RSV780 | Nanobody binding to | LG2076D | US20110182897 SEQ ID NO: | 25360 |
| | RSV F protein | | 226 | |
| RSV781 | Nanobody binding to | LG2076E | US20110182897 SEQ ID NO: | 25361 |
| | RSV F protein | | 227 | |
| RSV782 | Nanobody binding to | LG2076F | US20110182897 SEQ ID NO: | 25362 |
| | RSV F protein | | 228 | |
| RSV783 | Nanobody binding to | LG2079A | US20110182897 SEQ ID NO: | 25363 |
| | RSV F protein | | 229 | |
| RSV784 | Nanobody binding to | LG2079B | US20110182897 SEQ ID NO: | 25364 |
| | RSV F protein | | 230 | |
| RSV785 | Nanobody binding to | LG2079C | US20110182897 SEQ ID NO: | 25365 |
| | RSV F protein | | 231 | |
| RSV786 | Nanobody binding to | LG2079D | US20110182897 SEQ ID NO: | 25366 |
| | RSV F protein | | 232 | |
| RSV787 | Nanobody binding to | LG2079E | US20110182897 SEQ ID NO: | 25367 |
| | RSV F protein | | 233 | |
| RSV788 | Nanobody binding to | LG2079F | US20110182897 SEQ ID NO: | 25368 |
| | RSV F protein | | 234 | |
| RSV789 | Nanobody binding to | LG2079G | US20110182897 SEQ ID NO: | 25369 |
| | RSV F protein | | 235 | |
| RSV790 | Nanobody binding to | LG2079H | US20110182897 SEQ ID NO: | 25370 |
| | RSV F protein | | 236 | |
| RSV791 | Nanobody binding to | LG213B7 | US20110182897 SEQ ID NO: | 25371 |
| | RSV F protein | | 237 | |
| RSV792 | Nanobody binding to | LG213D6 | US20110182897 SEQ ID NO: | 25372 |
| | RSV F protein | | 238 | |
| RSV793 | Nanobody binding to | LG213D7 | US20110182897 SEQ ID NO: | 25373 |
| | RSV F protein | | 239 | |
| RSV794 | Nanobody binding to | LG213E6 | US20110182897 SEQ ID NO: | 25374 |
| | RSV F protein | | 240 | |
| RSV795 | Nanobody binding to | LG213H7 | US20110182897 SEQ ID NO: | 25375 |
| | RSV F protein | | 241 | |
| RSV796 | Nanobody binding to | LG214A8 | US20110182897 SEQ ID NO: | 25376 |
| | RSV F protein | | 242 | |
| RSV797 | Nanobody binding to | LG214C10 | US20110182897 SEQ ID NO: | 25377 |
| | RSV F protein | | 243 | |
| RSV798 | Nanobody binding to | LG214D10 | US20110182897 SEQ ID NO: | 25378 |
| | RSV F protein | | 244 | |
| RSV799 | Nanobody binding to | LG214E8 | US20110182897 SEQ ID NO: | 25379 |
| | RSV F protein | | 245 | |
| RSV800 | Nanobody binding to | LG214F8 | US20110182897 SEQ ID NO: | 25380 |
| | RSV F protein | | 246 | |
| RSV801 | Nanobody binding to | LG214H10 | US20110182897 SEQ ID NO: | 25381 |
| | RSV F protein | | 247 | |
| RSV802 | Nanobody binding to | RSVPMP5C1 | US20110182897 SEQ ID NO: | 25382 |
| | RSV F protein | | 248 | |
| RSV803 | Nanobody binding to | RSVPMP8A1 | US20110182897 SEQ ID NO: | 25383 |
| | RSV F protein | | 249 | |
| RSV804 | Nanobody binding to | RSVPMP8G1 | US20110182897 SEQ ID NO: | 25384 |
| | RSV F protein | | 250 | |
| RSV805 | Nanobody binding to | RSVPMP25B3 | US20110182897 SEQ ID NO: | 25385 |
| | RSV F protein | | 251 | |
| RSV806 | Nanobody binding to | RSVPMP8C8 | US20110182897 SEQ ID NO: | 25386 |
| | RSV F protein | | 252 | |
| RSV807 | Nanobody binding to | RSVPMP5A6 | US20110182897 SEQ ID NO: | 25387 |
| | RSV F protein | | 253 | |
| RSV808 | Nanobody binding to | RSVPMP8E11 | US20110182897 SEQ ID NO: | 25388 |
| | RSV F protein | | 254 | |
| RSV809 | Nanobody binding to | RSVPMP8F11 | US20110182897 SEQ ID NO: | 25389 |
| | RSV F protein | | 255 | |
| RSV810 | Nanobody binding to | RSVPMP13F11 | US20110182897 SEQ ID NO: | 25390 |
| | RSV F protein | | 256 | |
| RSV811 | Nanobody binding to | RSVPMP15B8 | US20110182897 SEQ ID NO: | 25391 |
| | RSV F protein | | 257 | |
| RSV812 | Nanobody binding to | RSVPMP15G11 | US20110182897 SEQ ID NO: | 25392 |
| | RSV F protein | | 258 | |
| RSV813 | Nanobody binding to | RSVPMP17C10 | US20110182897 SEQ ID NO: | 25393 |
| | RSV F protein | | 259 | |
| RSV814 | Nanobody binding to | RSVPMP21E7 | US20110182897 SEQ ID NO: | 25394 |
| | RSV F protein | | 260 | |
| RSV815 | Nanobody binding to | RSVPMP21F8 | US20110182897 SEQ ID NO: | 25395 |
| | RSV F protein | | 261 | |
| RSV816 | Nanobody binding to | RSVPMP5A2 | US20110182897 SEQ ID NO: | 25396 |
| | RSV F protein | | 262 | |
| RSV817 | Nanobody binding to | RSVPMP5B2 | US20110182897 SEQ ID NO: | 25397 |
| | RSV F protein | | 263 | |
| RSV818 | Nanobody binding to | RSVPMP5C3 | US20110182897 SEQ ID NO: | 25398 |
| | RSV F protein | | 264 | |
| RSV819 | Nanobody binding to | RSVPMP5D2 | US20110182897 SEQ ID NO: | 25399 |
| | RSV F protein | | 265 | |
| RSV820 | Nanobody binding to | RSVPMP5E2 | US20110182897 SEQ ID NO: | 25400 |
| | RSV F protein | | 266 | |
| RSV821 | Nanobody binding to | RSVPMP5F3 | US20110182897 SEQ ID NO: | 25401 |
| | RSV F protein | | 267 | |
| RSV822 | Nanobody binding to | RSVPMP5G3 | US20110182897 SEQ ID NO: | 25402 |
| | RSV F protein | | 268 | |
| RSV823 | Nanobody binding to | RSVPMP5H2 | US20110182897 SEQ ID NO: | 25403 |
| | RSV F protein | | 269 | |
| RSV824 | Nanobody binding to | RSVPMP5H3 | US20110182897 SEQ ID NO: | 25404 |
| | RSV F protein | | 270 | |
| RSV825 | Nanobody binding to | RSVPMP8C1 | US20110182897 SEQ ID NO: | 25405 |
| | RSV F protein | | 271 | |
| RSV826 | Nanobody binding to | RSVPMP8F2 | US20110182897 SEQ ID NO: | 25406 |
| | RSV F protein | | 272 | |
| RSV827 | Nanobody binding to | RSVPMP8G4 | US20110182897 SEQ ID NO: | 25407 |
| | RSV F protein | | 273 | |
| RSV828 | Nanobody binding to | RSVPMP13A1 | US20110182897 SEQ ID NO: | 25408 |
| | RSV F protein | | 274 | |
| RSV829 | Nanobody binding to | RSVPMP13A4 | US20110182897 SEQ ID NO: | 25409 |
| | RSV F protein | | 275 | |
| RSV830 | Nanobody binding to | RSVPMP13B1 | US20110182897 SEQ ID NO: | 25410 |
| | RSV F protein | | 276 | |
| RSV831 | Nanobody binding to | RSVPMP13B2 | US20110182897 SEQ ID NO: | 25411 |
| | RSV F protein | | 277 | |
| RSV832 | Nanobody binding to | RSVPMP13C1 | US20110182897 SEQ ID NO: | 25412 |
| | RSV F protein | | 278 | |
| RSV833 | Nanobody binding to | RSVPMP13C3 | US20110182897 SEQ ID NO: | 25413 |
| | RSV F protein | | 279 | |
| RSV834 | Nanobody binding to | RSVPMP13D6 | US20110182897 SEQ ID NO: | 25414 |
| | RSV F protein | | 280 | |
| RSV835 | Nanobody binding to | RSVPMP13E2 | US20110182897 SEQ ID NO: | 25415 |
| | RSV F protein | | 281 | |
| RSV836 | Nanobody binding to | RSVPMP13E3 | US20110182897 SEQ ID NO: | 25416 |
| | RSV F protein | | 282 | |
| RSV837 | Nanobody binding to | RSVPMP15A5 | US20110182897 SEQ ID NO: | 25417 |
| | RSV F protein | | 283 | |
| RSV838 | Nanobody binding to | RSVPMP15A6 | US20110182897 SEQ ID NO: | 25418 |
| | RSV F protein | | 284 | |
| RSV839 | Nanobody binding to | RSVPMP15B2 | US20110182897 SEQ ID NO: | 25419 |
| | RSV F protein | | 285 | |
| RSV840 | Nanobody binding to | RSVPMP15B3 | US20110182897 SEQ ID NO: | 25420 |
| | RSV F protein | | 286 | |
| RSV841 | Nanobody binding to | RSVPMP15E5 | US20110182897 SEQ ID NO: | 25421 |
| | RSV F protein | | 287 | |
| RSV842 | Nanobody binding to | RSVPMP17C2 | US20110182897 SEQ ID NO: | 25422 |
| | RSV F protein | | 288 | |
| RSV843 | Nanobody binding to | RSVPMP17D4 | US20110182897 SEQ ID NO: | 25423 |
| | RSV F protein | | 289 | |
| RSV844 | Nanobody binding to | RSVPMP17G4 | US20110182897 SEQ ID NO: | 25424 |
| | RSV F protein | | 290 | |
| RSV845 | Nanobody binding to | RSVPMP19B2 | US20110182897 SEQ ID NO: | 25425 |
| | RSV F protein | | 291 | |
| RSV846 | Nanobody binding to | RSVPMP25A4 | US20110182897 SEQ ID NO: | 25426 |
| | RSV F protein | | 292 | |
| RSV847 | Nanobody binding to | RSVPMP25A9 | US20110182897 SEQ ID NO: | 25427 |
| | RSV F protein | | 293 | |
| RSV848 | Nanobody binding to | RSVPMP25B5 | US20110182897 SEQ ID NO: | 25428 |
| | RSV F protein | | 294 | |
| RSV849 | Nanobody binding to | RSVPMP25G2 | US20110182897 SEQ ID NO: | 25429 |
| | RSV F protein | | 295 | |
| RSV850 | Nanobody binding to | RSVPMP25H5 | US20110182897 SEQ ID NO: | 25430 |
| | RSV F protein | | 296 | |
| RSV851 | Nanobody binding to | RSVPMP25E11 | US20110182897 SEQ ID NO: | 25431 |
| | RSV F protein | | 297 | |
| RSV852 | Nanobody binding to | RSVPMP8G3 | US20110182897 SEQ ID NO: | 25432 |
| | RSV F protein | | 298 | |
| RSV853 | Nanobody binding to | RSVPMP13B5 | US20110182897 SEQ ID NO: | 25433 |
| | RSV F protein | | 299 | |
| RSV854 | Nanobody binding to | RSVPMP15F2 | US20110182897 SEQ ID NO: | 25434 |
| | RSV F protein | | 300 | |
| RSV855 | Nanobody binding to | RSVPMP19E2 | US20110182897 SEQ ID NO: | 25435 |
| | RSV F protein | | 301 | |
| RSV856 | Nanobody binding to | RSVPMP25D1 | US20110182897 SEQ ID NO: | 25436 |
| | RSV F protein | | 302 | |
| RSV857 | Nanobody binding to | RSVPMP5A1 | US20110182897 SEQ ID NO: | 25437 |
| | RSV F protein | | 303 | |
| RSV858 | Nanobody binding to | RSVPMP5G2 | US20110182897 SEQ ID NO: | 25438 |
| | RSV F protein | | 304 | |
| RSV859 | Nanobody binding to | RSVPMP5H1 | US20110182897 SEQ ID NO: | 25439 |
| | RSV F protein | | 305 | |
| RSV860 | Nanobody binding to | RSVPMP6B1 | US20110182897 SEQ ID NO: | 25440 |
| | RSV F protein | | 306 | |
| RSV861 | Nanobody binding to | RSVPMP8H2 | US20110182897 SEQ ID NO: | 25441 |
| | RSV F protein | | 307 | |
| RSV862 | Nanobody binding to | RSVPMP8H3 | US20110182897 SEQ ID NO: | 25442 |
| | RSV F protein | | 308 | |
| RSV863 | Nanobody binding to | RSVPMP13A3 | US20110182897 SEQ ID NO: | 25443 |
| | RSV F protein | | 309 | |
| RSV864 | Nanobody binding to | RSVPMP13C5 | US20110182897 SEQ ID NO: | 25444 |
| | RSV F protein | | 310 | |
| RSV865 | Nanobody binding to | RSVPMP13H1 | US20110182897 SEQ ID NO: | 25445 |
| | RSV F protein | | 311 | |
| RSV866 | Nanobody binding to | RSVPMP13H2 | US20110182897 SEQ ID NO: | 25446 |
| | RSV F protein | | 312 | |
| RSV867 | Nanobody binding to | RSVPMP15E6 | US20110182897 SEQ ID NO: | 25447 |
| | RSV F protein | | 313 | |
| RSV868 | Nanobody binding to | RSVPMP17A3 | US20110182897 SEQ ID NO: | 25448 |
| | RSV F protein | | 314 | |
| RSV869 | Nanobody binding to | RSVPMP25G8 | US20110182897 SEQ ID NO: | 25449 |
| | RSV F protein | | 315 | |
| RSV870 | Nanobody binding to | RSVPMP6D1 | US20110182897 SEQ ID NO: | 25450 |
| | RSV F protein | | 316 | |
| RSV871 | Nanobody binding to | RSVPMP8D5 | US20110182897 SEQ ID NO: | 25451 |
| | RSV F protein | | 317 | |
| RSV872 | Nanobody binding to | RSVPMP13B4 | US20110182897 SEQ ID NO: | 25452 |
| | RSV F protein | | 318 | |
| RSV873 | Nanobody binding to | RSVPMP13B6 | US20110182897 SEQ ID NO: | 25453 |
| | RSV F protein | | 319 | |
| RSV874 | Nanobody binding to | RSVPMP13E6 | US20110182897 SEQ ID NO: | 25454 |
| | RSV F protein | | 320 | |
| RSV875 | Nanobody binding to | RSVPMP13F4 | US20110182897 SEQ ID NO: | 25455 |
| | RSV F protein | | 321 | |
| RSV876 | Nanobody binding to | RSVPMP15H3 | US20110182897 SEQ ID NO: | 25456 |
| | RSV F protein | | 322 | |
| RSV877 | Nanobody binding to | RSVPMP17E5 | US20110182897 SEQ ID NO: | 25457 |
| | RSV F protein | | 323 | |
| RSV878 | Nanobody binding to | RSVPMP19D3 | US20110182897 SEQ ID NO: | 25458 |
| | RSV F protein | | 324 | |
| RSV879 | Nanobody binding to | RSVPMP19F3 | US20110182897 SEQ ID NO: | 25459 |
| | RSV F protein | | 325 | |
| RSV880 | Nanobody binding to | RSVPMP25C4 | US20110182897 SEQ ID NO: | 25460 |
| | RSV F protein | | 326 | |
| RSV881 | Nanobody binding to | RSVPMP25E3 | US20110182897 SEQ ID NO: | 25461 |
| | RSV F protein | | 327 | |
| RSV882 | Nanobody binding to | RSVPMP5G4 | US20110182897 SEQ ID NO: | 25462 |
| | RSV F protein | | 328 | |
| RSV883 | Nanobody binding to | RSVPMP6G5 | US20110182897 SEQ ID NO: | 25463 |
| | RSV F protein | | 329 | |
| RSV884 | Nanobody binding to | RSVPMP8E6 | US20110182897 SEQ ID NO: | 25464 |
| | RSV F protein | | 330 | |
| RSV885 | Nanobody binding to | RSVPMP13A10 | US20110182897 SEQ ID NO: | 25465 |
| | RSV F protein | | 331 | |
| RSV886 | Nanobody binding to | RSVPMP21H10 | US20110182897 SEQ ID NO: | 25466 |
| | RSV F protein | | 332 | |
| RSV887 | Nanobody binding to | RSVPMP5A8 | US20110182897 SEQ ID NO: | 25467 |
| | RSV F protein | | 333 | |
| RSV888 | Nanobody binding to | RSVPMP5A10 | US20110182897 SEQ ID NO: | 25468 |
| | RSV F protein | | 334 | |
| RSV889 | Nanobody binding to | RSVPMP14A6 | US20110182897 SEQ ID NO: | 25469 |
| | RSV F protein | | 335 | |
| RSV890 | Nanobody binding to | RSVPMP16A6 | US20110182897 SEQ ID NO: | 25470 |
| | RSV F protein | | 336 | |
| RSV891 | Nanobody binding to | RSVPMP22D6 | US20110182897 SEQ ID NO: | 25471 |
| | RSV F protein | | 337 | |
| RSV892 | Nanobody binding to | RSVPMP8E2 | US20110182897 SEQ ID NO: | 25472 |
| | RSV F protein | | 338 | |
| RSV893 | Nanobody binding to | RSVPMP8C6 | US20110182897 SEQ ID NO: | 25473 |
| | RSV F protein | | 339 | |
| RSV894 | Nanobody binding to | RSVPMP5C6 | US20110182897 SEQ ID NO: | 25474 |
| | RSV F protein | | 340 | |
| RSV895 | Nanobody binding to | RSVPMP6D4 | US20110182897 SEQ ID NO: | 25475 |
| | RSV F protein | | 341 | |
| RSV896 | Nanobody binding to | RSVPMP8B10 | US20110182897 SEQ ID NO: | 25476 |
| | RSV F protein | | 342 | |
| RSV897 | Nanobody binding to | RSVPMP8E10 | US20110182897 SEQ ID NO: | 25477 |
| | RSV F protein | | 343 | |
| RSV898 | Nanobody binding to | RSVPMP15A7 | US20110182897 SEQ ID NO: | 25478 |
| | RSV F protein | | 344 | |
| RSV899 | Nanobody binding to | RSVPMP15E10 | US20110182897 SEQ ID NO: | 25479 |
| | RSV F protein | | 345 | |
| RSV900 | Nanobody binding to | RSVPMP13C7 | US20110182897 SEQ ID NO: | 25480 |
| | RSV F protein | | 346 | |
| RSV901 | Nanobody binding to | RSVPMP15A9 | US20110182897 SEQ ID NO: | 25481 |
| | RSV F protein | | 347 | |
| RSV902 | Nanobody binding to | RSVPMP15F11 | US20110182897 SEQ ID NO: | 25482 |
| | RSV F protein | | 348 | |
| RSV903 | Nanobody binding to | RSVPMP15A1 | US20110182897 SEQ ID NO: | 25483 |
| | RSV F protein | | 349 | |
| RSV904 | Nanobody binding to | RSVPMP6H2 | US20110182897 SEQ ID NO: | 25484 |
| | RSV F protein | | 350 | |
| RSV905 | Nanobody binding to | RSVPMP17A9 | US20110182897 SEQ ID NO: | 25485 |
| | RSV F protein | | 351 | |
| RSV906 | Nanobody binding to | RSVPMP7G1 | US20110182897 SEQ ID NO: | 25486 |
| | RSV F protein | | 352 | |
| RSV907 | Nanobody binding to | RSVPMP5A9 | US20110182897 SEQ ID NO: | 25487 |
| | RSV F protein | | 353 | |
| RSV908 | Nanobody binding to | RSVPMP7B2 | US20110182897 SEQ ID NO: | 25488 |
| | RSV F protein | | 354 | |
| RSV909 | Nanobody binding to | RSVPMP22A4 | US20110182897 SEQ ID NO: | 25489 |
| | RSV F protein | | 355 | |
| RSV910 | Nanobody binding to | RSVPMP22E10 | US20110182897 SEQ ID NO: | 25490 |
| | RSV F protein | | 356 | |
| RSV911 | Nanobody binding to | RSVPMP22H4 | US20110182897 SEQ ID NO: | 25491 |
| | RSV F protein | | 357 | |
| RSV912 | Nanobody binding to | RSVPMP15C5 | US20110182897 SEQ ID NO: | 25492 |
| | RSV F protein | | 358 | |
| RSV913 | Nanobody binding to | RSVNC39 | US20110182897 SEQ ID NO: | 25493 |
| | RSV F protein | | 359 | |
| RSV914 | Nanobody binding to | RSVPMP7B9 | US20110182897 SEQ ID NO: | 25494 |
| | RSV F protein | | 360 | |
| RSV915 | Nanobody binding to | RSVPMP15E11 | US20110182897 SEQ ID NO: | 25495 |
| | RSV F protein | | 361 | |
| RSV916 | Nanobody binding to | RSVPMP7E7 | US20110182897 SEQ ID NO: | 25496 |
| | RSV F protein | | 362 | |
| RSV917 | Nanobody binding to | RSVPMP14H3 | US20110182897 SEQ ID NO: | 25497 |
| | RSV F protein | | 363 | |
| RSV918 | Nanobody binding to | RSVPMP24D6 | US20110182897 SEQ ID NO: | 25498 |
| | RSV F protein | | 364 | |
| RSV919 | Nanobody binding to | RSVPMP23E5 | US20110182897 SEQ ID NO: | 25499 |
| | RSV F protein | | 365 | |
| RSV920 | Nanobody binding to | RSVPMP8A6 | US20110182897 SEQ ID NO: | 25500 |
| | RSV F protein | | 366 | |
| RSV921 | Nanobody binding to | RSVPMP14E2 | US20110182897 SEQ ID NO: | 25501 |
| | RSV F protein | | 367 | |
| RSV922 | Nanobody binding to | RSVPMP25F3 | US20110182897 SEQ ID NO: | 25502 |
| | RSV F protein | | 368 | |
| RSV923 | Nanobody binding to | RSVPMP19A6 | US20110182897 SEQ ID NO: | 25503 |
| | RSV F protein | | 369 | |
| RSV924 | Nanobody binding to | RSVPMP23G1 | US20110182897 SEQ ID NO: | 25504 |
| | RSV F protein | | 370 | |
| RSV925 | Nanobody binding to | RSVPMP15H8 | US20110182897 SEQ ID NO: | 25505 |
| | RSV F protein | | 371 | |
| RSV926 | Nanobody binding to | RSVNC41 | US20110182897 SEQ ID NO: | 25506 |
| | RSV F protein | | 372 | |
| RSV927 | Nanobody binding to | RSVPMP6A8 | US20110182897 SEQ ID NO: | 25507 |
| | RSV F protein | | 373 | |
| RSV928 | Nanobody binding to | RSVPMP25H9 | US20110182897 SEQ ID NO: | 25508 |
| | RSV F protein | | 374 | |
| RSV929 | Nanobody binding to | RSVPMP8B11 | US20110182897 SEQ ID NO: | 25509 |
| | RSV F protein | | 375 | |
| RSV930 | Nanobody binding to | RSVPMP17E1 | US20110182897 SEQ ID NO: | 25510 |
| | RSV F protein | | 376 | |
| RSV931 | Nanobody binding to | RSVPMP21A4 | US20110182897 SEQ ID NO: | 25511 |
| | RSV F protein | | 377 | |
| RSV932 | Nanobody binding to | RSVPMP25A11 | US20110182897 SEQ ID NO: | 25512 |
| | RSV F protein | | 378 | |
| RSV933 | Nanobody binding to | RSVPMP25C8 | US20110182897 SEQ ID NO: | 25513 |
| | RSV F protein | | 379 | |
| RSV934 | Nanobody binding to | RSVNC23 | US20110182897 SEQ ID NO: | 25514 |
| | RSV F protein | | 380 | |
| RSV935 | Nanobody binding to | RSVPMP20A11 | US20110182897 SEQ ID NO: | 25515 |
| | RSV F protein | | 381 | |
| RSV936 | Nanobody binding to | RSVPMP20A9 | US20110182897 SEQ ID NO: | 25516 |
| | RSV F protein | | 382 | |
| RSV937 | Nanobody binding to | RSVPMP1F7 | US20110182897 SEQ ID NO: | 25517 |
| | RSV F protein | | 383 | |
| RSV938 | Nanobody binding to | RSVPMP20D6 | US20110182897 SEQ ID NO: | 25518 |
| | RSV F protein | | 384 | |
| RSV939 | Nanobody binding to | RSVPMP1F1 | US20110182897 SEQ ID NO: | 25519 |
| | RSV F protein | | 385 | |
| RSV940 | Nanobody binding to | RSVPMP3D3 | US20110182897 SEQ ID NO: | 25520 |
| | RSV F protein | | 386 | |
| RSV941 | Nanobody binding to | RSVPMP3E6 | US20110182897 SEQ ID NO: | 25521 |
| | RSV F protein | | 387 | |
| RSV942 | Nanobody binding to | RSVPMP1C8 | US20110182897 SEQ ID NO: | 25522 |
| | RSV F protein | | 388 | |
| RSV943 | Nanobody binding to | RSVPMP1A2 | US20110182897 SEQ ID NO: | 25523 |
| | RSV F protein | | 389 | |
| RSV944 | Nanobody binding to | RSVPMP1C5 | US20110182897 SEQ ID NO: | 25524 |
| | RSV F protein | | 390 | |
| RSV945 | Nanobody binding to | RSVPMP20G5 | US20110182897 SEQ ID NO: | 25525 |
| | RSV F protein | | 391 | |
| RSV946 | Nanobody binding to | RSVPMP4D8 | US20110182897 SEQ ID NO: | 25526 |
| | RSV F protein | | 392 | |
| RSV947 | Nanobody binding to | RSVPMP20B6 | US20110182897 SEQ ID NO: | 25527 |
| | RSV F protein | | 393 | |
| RSV948 | Nanobody binding to | RSVPMPID11 | US20110182897 SEQ ID NO: | 25528 |
| | RSV F protein | | 394 | |
| RSV949 | Nanobody binding to | RSVPMP20A8 | US20110182897 SEQ ID NO: | 25529 |
| | RSV F protein | | 395 | |
| RSV950 | Nanobody binding to | RSVPMP20E7 | US20110182897 SEQ ID NO: | 25530 |
| | RSV F protein | | 396 | |
| RSV951 | Nanobody binding to | RSVPMP20G8 | US20110182897 SEQ ID NO: | 25531 |
| | RSV F protein | | 397 | |
| RSV952 | Nanobody binding to | RSVPMP2D3 | US20110182897 SEQ ID NO: | 25532 |
| | RSV F protein | | 398 | |
| RSV953 | Nanobody binding to | RSVPMP2G5 | US20110182897 SEQ ID NO: | 25533 |
| | RSV F protein | | 399 | |
| RSV954 | Nanobody binding to | RSVPMP2A6 | US20110182897 SEQ ID NO: | 25534 |
| | RSV F protein | | 400 | |
| RSV955 | Nanobody binding to | RSVPMP3A2 | US20110182897 SEQ ID NO: | 25535 |
| | RSV F protein | | 401 | |
| RSV956 | Nanobody binding to | RSVPMP4A8 | US20110182897 SEQ ID NO: | 25536 |
| | RSV F protein | | 402 | |
| RSV957 | Nanobody binding to | RSVPMP4F9 | US20110182897 SEQ ID NO: | 25537 |
| | RSV F protein | | 403 | |
| RSV958 | Nanobody binding to | RSVPMP1A6 | US20110182897 SEQ ID NO: | 25538 |
| | RSV F protein | | 404 | |
| RSV959 | Nanobody binding to | RSVPMP3C2 | US20110182897 SEQ ID NO: | 25539 |
| | RSV F protein | | 405 | |
| RSV960 | Nanobody binding to | RSVPMP4H9 | US20110182897 SEQ ID NO: | 25540 |
| | RSV F protein | | 406 | |
| RSV961 | Nanobody binding to | RSVPMP4B10 | US20110182897 SEQ ID NO: | 25541 |
| | RSV F protein | | 407 | |
| RSV962 | Nanobody binding to | 203B1 | US20110182897 SEQ ID NO: | 25542 |
| | RSV F protein | | 2431 | |
| RSV963 | Nanobody binding to | 203B2 | US20110182897 SEQ ID NO: | 25543 |
| | RSV F protein | | 2432 | |
| RSV964 | Nanobody binding to | 203G1 | US20110182897 SEQ ID NO: | 25544 |
| | RSV F protein | | 2433 | |
| RSV965 | Nanobody binding to | 203H1 | US20110182897 SEQ ID NO: | 25545 |
| | RSV F protein | | 2434 | |
| RSV966 | Nanobody binding to | 202E4 | US20110182897 SEQ ID NO: | 25546 |
| | RSV F protein | | 2435 | |
| RSV967 | Nanobody binding to | 189E2 | US20110182897 SEQ ID NO: | 25547 |
| | RSV F protein | | 2436 | |
| RSV968 | Nanobody binding to | 203A12 | US20110182897 SEQ ID NO: | 25548 |
| | RSV F protein | | 2437 | |
| RSV969 | Nanobody binding to | 203A9 | US20110182897 SEQ ID NO: | 25549 |
| | RSV F protein | | 2438 | |
| RSV970 | Nanobody binding to | 203B12 | US20110182897 SEQ ID NO: | 25550 |
| | RSV F protein | | 2439 | |
| RSV971 | Nanobody binding to | 203D2 | US20110182897 SEQ ID NO: | 25551 |
| | RSV F protein | | 2440 | |
| RSV972 | Nanobody binding to | 203D9 | US20110182897 SEQ ID NO: | 25552 |
| | RSV F protein | | 2441 | |
| RSV973 | Nanobody binding to | 203G3 | US20110182897 SEQ ID NO: | 25553 |
| | RSV F protein | | 2442 | |
| RSV974 | Nanobody binding to | 203G9 | US20110182897 SEQ ID NO: | 25554 |
| | RSV F protein | | 2443 | |
| RSV975 | Nanobody binding to | 203G10 | US20110182897 SEQ ID NO: | 25555 |
| | RSV F protein | | 2444 | |
| RSV976 | Nanobody binding to | 203H9 | US20110182897 SEQ ID NO: | 25556 |
| | RSV F protein | | 2445 | |
| RSV977 | Nanobody binding to | 203H10 | US20110182897 SEQ ID NO: | 25557 |
| | RSV F protein | | 2446 | |
| RSV978 | Nanobody binding to | 202E4 | US20110182897 SEQ ID NO: | 25558 |
| | RSV F protein | | 2447 | |
| RSV979 | Nanobody binding to | 189E2 | US20110182897 SEQ ID NO: | 25559 |
| | RSV F protein | | 2448 | |
| RSV980 | Nanobody binding to | PRSVPMP20C3 | US20110182897 SEQ ID NO: | 25560 |
| | RSV F protein | | 2574 | |
| RSV981 | Nanobody binding to | PRSVPMP20C5 | US20110182897 SEQ ID NO: | 25561 |
| | RSV F protein | | 2575 | |
| RSV982 | Nanobody binding to | PRSVPMP20B2 | US20110182897 SEQ ID NO: | 25562 |
| | RSV F protein | | 2576 | |
| RSV983 | Nanobody binding to | PRSVPMP20C1 | US20110182897 SEQ ID NO: | 25563 |
| | RSV F protein | | 2577 | |
| RSV984 | Nanobody binding to | PRSVPMP1G8 | US20110182897 SEQ ID NO: | 25564 |
| | RSV F protein | | 2578 | |
| RSV985 | Nanobody binding to | PRSVNMP1A4 | US20110182897 SEQ ID NO: | 25565 |
| | RSV F protein | | 2579 | |
| RSV986 | Nanobody binding to | PRSVPMP13E12 | US20110182897 SEQ ID NO: | 25566 |
| | RSV F protein | | 2580 | |
| RSV987 | Nanobody binding to | PRSVPMP5C6 | US20110182897 SEQ ID NO: | 25567 |
| | RSV F protein | | 2581 | |
| RSV988 | Nanobody binding to | LG203E7 | US20110182897 SEQ ID NO: | 25568 |
| | RSV F protein | | 2682 | |
| RSV989 | Nanobody binding to | LG203G8 | US20110182897 SEQ ID NO: | 25569 |
| | RSV F protein | | 2683 | |
| RSV990 | Nanobody binding to | LG211A10 | US20110182897 SEQ ID NO: | 25570 |
| | RSV F protein | | 2684 | |
| RSV991 | Nanobody binding to | LG211A8 | US20110182897 SEQ ID NO: | 25571 |
| | RSV F protein | | 2685 | |
| RSV992 | Nanobody binding to | LG211B10 | US20110182897 SEQ ID NO: | 25572 |
| | RSV F protein | | 2686 | |
| RSV993 | Nanobody binding to | LG211B8 | US20110182897 SEQ ID NO: | 25573 |
| | RSV F protein | | 2687 | |
| RSV994 | Nanobody binding to | LG211C12 | US20110182897 SEQ ID NO: | 25574 |
| | RSV F protein | | 2688 | |
| RSV995 | Nanobody binding to | LG211C8 | US20110182897 SEQ ID NO: | 25575 |
| | RSV F protein | | 2689 | |
| RSV996 | Nanobody binding to | LG211D10 | US20110182897 SEQ ID NO: | 25576 |
| | RSV F protein | | 2690 | |
| RSV997 | Nanobody binding to | LG211D8 | US20110182897 SEQ ID NO: | 25577 |
| | RSV F protein | | 2691 | |
| RSV998 | Nanobody binding to | LG211E10 | US20110182897 SEQ ID NO: | 25578 |
| | RSV F protein | | 2692 | |
| RSV999 | Nanobody binding to | LG211E12 | US20110182897 SEQ ID NO: | 25579 |
| | RSV F protein | | 2693 | |
| RSV1000 | Nanobody binding to | LG211E8 | US20110182897 SEQ ID NO: | 25580 |
| | RSV F protein | | 2694 | |
| RSV1001 | Nanobody binding to | LG211H8 | US20110182897 SEQ ID NO: | 25581 |
| | RSV F protein | | 2695 | |
| RSV1002 | Nanobody binding to | LG212A10 | US20110182897 SEQ ID NO: | 25582 |
| | RSV F protein | | 2696 | |
| RSV1003 | Nanobody binding to | LG212A12 | US20110182897 SEQ ID NO: | 25583 |
| | RSV F protein | | 2697 | |
| RSV1004 | Nanobody binding to | LG212A2 | US20110182897 SEQ ID NO: | 25584 |
| | RSV F protein | | 2698 | |
| RSV1005 | Nanobody binding to | LG212A8 | US20110182897 SEQ ID NO: | 25585 |
| | RSV F protein | | 2699 | |
| RSV1006 | Nanobody binding to | LG212B12 | US20110182897 SEQ ID NO: | 25586 |
| | RSV F protein | | 2700 | |
| RSV1007 | Nanobody binding to | LG212B2 | US20110182897 SEQ ID NO: | 25587 |
| | RSV F protein | | 2701 | |
| RSV1008 | Nanobody binding to | LG212C12 | US20110182897 SEQ ID NO: | 25588 |
| | RSV F protein | | 2702 | |
| RSV1009 | Nanobody binding to | LG212D10 | US20110182897 SEQ ID NO: | 25589 |
| | RSV F protein | | 2703 | |
| RSV1010 | Nanobody binding to | LG212D12 | US20110182897 SEQ ID NO: | 25590 |
| | RSV F protein | | 2704 | |
| RSV1011 | Nanobody binding to | LG212D2 | US20110182897 SEQ ID NO: | 25591 |
| | RSV F protein | | 2705 | |
| RSV1012 | Nanobody binding to | LG212E10 | US20110182897 SEQ ID NO: | 25592 |
| | RSV F protein | | 2706 | |
| RSV1013 | Nanobody binding to | LG212E12 | US20110182897 SEQ ID NO: | 25593 |
| | RSV F protein | | 2707 | |
| RSV1014 | Nanobody binding to | LG212E6 | US20110182897 SEQ ID NO: | 25594 |
| | RSV F protein | | 2708 | |
| RSV1015 | Nanobody binding to | LG212F10 | US20110182897 SEQ ID NO: | 25595 |
| | RSV F protein | | 2709 | |
| RSV1016 | Nanobody binding to | LG212F12 | US20110182897 SEQ ID NO: | 25596 |
| | RSV F protein | | 2710 | |
| RSV1017 | Nanobody binding to | LG212F6 | US20110182897 SEQ ID NO: | 25597 |
| | RSV F protein | | 2711 | |
| RSV1018 | Nanobody binding to | LG212F8 | US20110182897 SEQ ID NO: | 25598 |
| | RSV F protein | | 2712 | |
| RSV1019 | Nanobody binding to | LG212G10 | US20110182897 SEQ ID NO: | 25599 |
| | RSV F protein | | 2713 | |
| RSV1020 | Nanobody binding to | LG212G2 | US20110182897 SEQ ID NO: | 25600 |
| | RSV F protein | | 2714 | |
| RSV1021 | Nanobody binding to | LG212H10 | US20110182897 SEQ ID NO: | 25601 |
| | RSV F protein | | 2715 | |
| RSV1022 | Nanobody binding to | LG212H2 | US20110182897 SEQ ID NO: | 25602 |
| | RSV F protein | | 2716 | |
| RSV1023 | Nanobody binding to | LG212H8 | US20110182897 SEQ ID NO: | 25603 |
| | RSV F protein | | 2717 | |
| RSV1024 | Nanobody binding to | IV121 | US20110182897 SEQ ID NO: | 25604 |
| | RSV F protein | | 3064 | |
| RSV1025 | Nanobody binding to | IV122 | US20110182897 SEQ ID NO: | 25605 |
| | RSV F protein | | 3065 | |
| RSV1026 | Nanobody binding to | IV123 | US20110182897 SEQ ID NO: | 25606 |
| | RSV F protein | | 3066 | |
| RSV1027 | Nanobody binding to | IV126 | US20110182897 SEQ ID NO: | 25607 |
| | RSV F protein | | 3067 | |
| RSV1028 | Nanobody binding to | IV127 | US20110182897 SEQ ID NO: | 25608 |
| | RSV F protein | | 3068 | |
| RSV1029 | Nanobody binding to | IV131 | US20110182897 SEQ ID NO: | 25609 |
| | RSV F protein | | 3069 | |
| RSV1030 | Nanobody binding to | IV132 | US20110182897 SEQ ID NO: | 25610 |
| | RSV F protein | | 3070 | |
| RSV1031 | Nanobody binding to | IV133 | US20110182897 SEQ ID NO: | 25611 |
| | RSV F protein | | 3071 | |
| RSV1032 | Nanobody binding to | IV134 | US20110182897 SEQ ID NO: | 25612 |
| | RSV F protein | | 3072 | |
| RSV1033 | Nanobody binding to | IV135 | US20110182897 SEQ ID NO: | 25613 |
| | RSV F protein | | 3073 | |
| RSV1034 | Nanobody binding to | IV136 | US20110182897 SEQ ID NO: | 25614 |
| | RSV F protein | | 3074 | |
| RSV1035 | Nanobody binding to | IV140 | US20110182897 SEQ ID NO: | 25615 |
| | RSV F protein | | 3075 | |
| RSV1036 | Nanobody binding to | IV144 | US20110182897 SEQ ID NO: | 25616 |
| | RSV F protein | | 3076 | |
| RSV1037 | Nanobody binding to | IV156 | US20110182897 SEQ ID NO: | 25617 |
| | RSV F protein | | 3077 | |
| RSV1038 | Nanobody binding to | IV157 | US20110182897 SEQ ID NO: | 25618 |
| | RSV F protein | | 3078 | |
| RSV1039 | Nanobody binding to | IV160 | US20110182897 SEQ ID NO: | 25619 |
| | RSV F protein | | 3079 | |
| RSV1040 | Nanobody binding to | IV124 | US20110182897 SEQ ID NO: | 25620 |
| | RSV F protein | | 3080 | |
| RSV1041 | Nanobody binding to | IV125 | US20110182897 SEQ ID NO: | 25621 |
| | RSV F protein | | 3081 | |
| RSV1042 | Nanobody binding to | IV145 | US20110182897 SEQ ID NO: | 25622 |
| | RSV F protein | | 3082 | |
| RSV1043 | Nanobody binding to | IV146 | US20110182897 SEQ ID NO: | 25623 |
| | RSV F protein | | 3083 | |
| RSV1044 | Nanobody binding to | IV147 | US20110182897 SEQ ID NO: | 25624 |
| | RSV F protein | | 3084 | |
| RSV1045 | Nanobody binding to | IV151 | US20110182897 SEQ ID NO: | 25625 |
| | RSV F protein | | 3085 | |
| RSV1046 | Nanobody binding to | IV153 | US20110182897 SEQ ID NO: | 25626 |
| | RSV F protein | | 3086 | |
| RSV1047 | Nanobody binding to | IV154 | US20110182897 SEQ ID NO: | 25627 |
| | RSV F protein | | 3087 | |
| RSV1048 | Nanobody binding to | IV155 | US20110182897 SEQ ID NO: | 25628 |
| | RSV F protein | | 3088 | |
| RSV1049 | Nanobody binding to | IV1 | US20110182897 SEQ ID NO: | 25629 |
| | RSV F protein | | 3089 | |
| RSV1050 | Nanobody binding to | IV2 | US20110182897 SEQ ID NO: | 25630 |
| | RSV F protein | | 3090 | |
| RSV1051 | Nanobody binding to | IV3 | US20110182897 SEQ ID NO: | 25631 |
| | RSV F protein | | 3091 | |
| RSV1052 | Nanobody binding to | IV4 | US20110182897 SEQ ID NO: | 25632 |
| | RSV F protein | | 3092 | |
| RSV1053 | Nanobody binding to | IV6 | US20110182897 SEQ ID NO: | 25633 |
| | RSV F protein | | 3093 | |
| RSV1054 | Nanobody binding to | IV7 | US20110182897 SEQ ID NO: | 25634 |
| | RSV F protein | | 3094 | |
| RSV1055 | Nanobody binding to | IV9 | US20110182897 SEQ ID NO: | 25635 |
| | RSV F protein | | 3095 | |
| RSV1056 | Nanobody binding to | IV10 | US20110182897 SEQ ID NO: | 25636 |
| | RSV F protein | | 3096 | |
| RSV1057 | Nanobody binding to | IV11 | US20110182897 SEQ ID NO: | 25637 |
| | RSV F protein | | 3097 | |
| RSV1058 | Nanobody binding to | IV12 | US20110182897 SEQ ID NO: | 25638 |
| | RSV F protein | | 3098 | |
| RSV1059 | Nanobody binding to | IV16 | US20110182897 SEQ ID NO: | 25639 |
| | RSV F protein | | 3099 | |
| RSV1060 | Nanobody binding to | IV24 | US20110182897 SEQ ID NO: | 25640 |
| | RSV F protein | | 3100 | |
| RSV1061 | Nanobody binding to | IV26 | US20110182897 SEQ ID NO: | 25641 |
| | RSV F protein | | 3101 | |
| RSV1062 | Nanobody binding to | IV30 | US20110182897 SEQ ID NO: | 25642 |
| | RSV F protein | | 3102 | |
| RSV1063 | Nanobody binding to | IV34 | US20110182897 SEQ ID NO: | 25643 |
| | RSV F protein | | 3103 | |
| RSV1064 | Nanobody binding to | IV14 | US20110182897 SEQ ID NO: | 25644 |
| | RSV F protein | | 3104 | |
| RSV1065 | Nanobody binding to | IV15 | US20110182897 SEQ ID NO: | 25645 |
| | RSV F protein | | 3105 | |
| RSV1066 | Nanobody binding to | IV17 | US20110182897 SEQ ID NO: | 25646 |
| | RSV F protein | | 3106 | |
| RSV1067 | Nanobody binding to | IV18 | US20110182897 SEQ ID NO: | 25647 |
| | RSV F protein | | 3107 | |
| RSV1068 | Nanobody binding to | IV29 | US20110182897 SEQ ID NO: | 25648 |
| | RSV F protein | | 3108 | |
| RSV1069 | Nanobody binding to | IV31 | US20110182897 SEQ ID NO: | 25649 |
| | RSV F protein | | 3109 | |
| RSV1070 | Nanobody binding to | IV33 | US20110182897 SEQ ID NO: | 25650 |
| | RSV F protein | | 3110 | |
| RSV1071 | Nanobody binding to | IV35 | US20110182897 SEQ ID NO: | 25651 |
| | RSV F protein | | 3111 | |
| RSV1072 | Nanobody binding to | IV36 | US20110182897 SEQ ID NO: | 25652 |
| | RSV F protein | | 3112 | |
| RSV1073 | Nanobody binding to | IV40 | US20110182897 SEQ ID NO: | 25653 |
| | RSV F protein | | 3113 | |
| RSV1074 | Nanobody binding to | IV42 | US20110182897 SEQ ID NO: | 25654 |
| | RSV F protein | | 3114 | |
| RSV1075 | Nanobody binding to | IV8 | US20110182897 SEQ ID NO: | 25655 |
| | RSV F protein | | 3115 | |
| RSV1076 | Nanobody binding to | IV21 | US20110182897 SEQ ID NO: | 25656 |
| | RSV F protein | | 3116 | |
| RSV1077 | Nanobody binding to | IV23 | US20110182897 SEQ ID NO: | 25657 |
| | RSV F protein | | 3117 | |
| RSV1078 | Nanobody binding to | IV45 | US20110182897 SEQ ID NO: | 25658 |
| | RSV F protein | | 3118 | |
| RSV1079 | Nanobody binding to | IV47 | US20110182897 SEQ ID NO: | 25659 |
| | RSV F protein | | 3119 | |
| RSV1080 | Nanobody binding to | IV48 | US20110182897 SEQ ID NO: | 25660 |
| | RSV F protein | | 3120 | |
| RSV1081 | Nanobody binding to | IV50 | US20110182897 SEQ ID NO: | 25661 |
| | RSV F protein | | 3121 | |
| RSV1082 | Nanobody binding to | IV22 | US20110182897 SEQ ID NO: | 25662 |
| | RSV F protein | | 3122 | |
| RSV1083 | Nanobody binding to | IV37 | US20110182897 SEQ ID NO: | 25663 |
| | RSV F protein | | 3123 | |
| RSV1084 | Nanobody binding to | IV38 | US20110182897 SEQ ID NO: | 25664 |
| | RSV F protein | | 3124 | |
| RSV1085 | Nanobody binding to | IV5 | US20110182897 SEQ ID NO: | 25665 |
| | RSV F protein | | 3125 | |
| RSV1086 | Nanobody binding to | IV27 | US20110182897 SEQ ID NO: | 25666 |
| | RSV F protein | | 3126 | |
| RSV1087 | Nanobody binding to | IV25 | US20110182897 SEQ ID NO: | 25667 |
| | RSV F protein | | 3127 | |
| RSV1088 | Nanobody binding to | IV28 | US20110182897 SEQ ID NO: | 25668 |
| | RSV F protein | | 3128 | |

In some embodiments, the payload region of the viral particle comprises one or more nucleic acid sequences, fragments or variants thereof or encodes one or more polypeptides, fragments or variants thereof described in US Publication No. US20140363427, and international Publication No. WO2004083373, the contents of each of which are herein incorporated by reference in their entirety, against RSV F or RSV G protein.

In some embodiments, the payload region of the viral particle comprises one or more nucleic acid sequences encoding one or more of the payload antibody polypeptides listed in Table 34 against Hepatitis B, Hepatitis C and/or Hepatitis D (HEPBD1-HEPBD317; SEQ ID NO: 25669-25985).

TABLE 23
Antibodies against Hepatitis B, C, D viruses

| Antibody | | Antibody | | SEQ |
|---|---|---|---|---|
| No. | Description | Name | Reference Information | ID NO |
| HEPBD1 | Anti-preS1 | | Park, S. G., et al., Hepatitis B virus- | 25669 |
| | immunoglobulin, | | neutralizing anti-pre-S1 human antibody | |
| | HBV Ab | | fragments from large naive antibody phage | |
| | | | library; Antiviral Res. 68 (3), 109-115 | |
| | | | (2005); NCBI Accession # AAW82034.1 | |
| | | | (107aa) | |
| HEPBD2 | Anti-preS1 | | Park, S. G., et al., Hepatitis B virus- | 25670 |
| | immunoglobulin, | | neutralizing anti-pre-S1 human antibody | |
| | HBV Ab | | fragments from large naive antibody phage | |
| | | | library; Antiviral Res. 68 (3), 109-115 | |
| | | | (2005); NCBI Accession # AAW82035.1 | |
| | | | (132aa) | |
| HEPBD3 | Anti-preS1 | | Park, S. G., et al., Hepatitis B virus- | 25671 |
| | immunoglobulin, | | neutralizing anti-pre-S1 human antibody | |
| | HBV Ab | | fragments from large naive antibody phage | |
| | | | library; Antiviral Res. 68 (3), 109-115 | |
| | | | (2005); NCBI Accession # | |
| | | | AAW82033.1(111aa) | |
| HEPBD4 | Anti-preS1 | | Park, S. G., et al., Hepatitis B virus- | 25672 |
| | immunoglobulin, | | neutralizing anti-pre-S1 human antibody | |
| | HBV Ab | | fragments from large naive antibody phage | |
| | | | library; Antiviral Res. 68 (3), 109-115 | |
| | | | (2005); NCBI Accession # AAW82032.1 | |
| | | | (142aa) | |
| HEPBD5 | HCV Ab | Hu5b3.v3 | Pantua, H., et al., Glycan shifting on | 25673 |
| | | | hepatitis C virus(HCV) e2 glycoprotein is | |
| | | | a mechanism for escape from broadly | |
| | | | neutralizing antibodies; J. Mol. Biol. 425 | |
| | | | (11), 1899-1914 (2013) NCBI Accession # | |
| | | | 4HS8_H(228aa) | |
| HEPBD6 | HCV Ab | Igh526 | Kong L., et al., Structure of Hepatitis C | 25674 |
| | | | Virus Envelope Glycoprotein E1 Antigenic | |
| | | | Site 314-324 in Complex with Antibody | |
| | | | IGH526; J. Mol. Biol. 427 (16), 2617-2628 | |
| | | | (2015) NCBI Accession # | |
| | | | 4N0Y_H(231aa) | |
| HEPBD7 | Heavy chain partial, | | Esposito, G., et al., Recombinant human | 25675 |
| | HCV Ab | | antibodies specific for hepatitis C virus | |
| | | | proteins; Arch. Virol. 142 (3), 601-610 | |
| | | | (1997) NCBI Accession # CAA54914 | |
| | | | (122aa) | |
| HEPBD8 | Heavy chain variable | | EP0521348 | 25676 |
| | gene, Chimeric HBV | | | |
| | Ab | | | |
| HEPBD9 | Heavy chain variable | | Keck, Z. Y., et al., Human monoclonal | 25677 |
| | region partial, HCV | | antibody to hepatitis C virUSE1 | |
| | Ab | | glycoprotein that blocks virus attachment | |
| | | | and viral infectivity; J. Virol. 78 (13), | |
| | | | 7257-7263 (2004) NCBI Accession # | |
| | | | AAS47839 (142aa) | |
| HEPBD10 | Heavy chain variable | E183/A2 | US20120308580 SEQ ID NO: 33; | 25678 |
| | region, HBV Ab | | WO2011062562; CN102781961, EP2501723 | |
| HEPBD11 | Heavy chain variable | | US20100260712 SEQ ID NO: 1; | 25679 |
| | region, HBV Ab | | WO2009069917 | |
| HEPBD12 | Heavy chain variable | | WO2015107126 SEQ ID NO: 2 | 25680 |
| | region, HBV Ab | | | |
| HEPBD13 | Heavy chain variable | HB48-33, | U.S. Pat. No. 8,840,895 SEQ ID NO: 1 | 25681 |
| | region, HBV Ab | HB48-35, | | |
| | | HB48-59 | | |
| HEPBD14 | Heavy chain variable | HFW141 | U.S. Pat. No. 7,435,414 SEQ ID NO: 35; | 25682 |
| | region, HBV Ab | | US20060014937; WO2005100400; | |
| | | | CN1980956 | |
| HEPBD15 | Heavy chain variable | | U.S. Pat. No. 7,112,664 SEQ ID NO: 8; U.S. Pat. No. 6,680,053, | 25683 |
| | region, HBV Ab | | U.S. Pat. No. 6,924,368, US20020061581, | |
| | | | US20040191259, US20050249753, | |
| | | | WO2001092529 | |
| HEPBD16 | Heavy chain variable | Ab17.1.4 1 | USRE39586 SEQ ID NO: 4; U.S. Pat. No. 6,146,629; | 25684 |
| | region, HBV Ab | | WO1997047653 | |
| HEPBD17 | Heavy chain variable | Ab 19.79.5 | USRE40831 SEQ ID NO: 4 | 25685 |
| | region, HBV Ab | | | |
| HEPBD18 | Heavy chain variable | | US20150232537 SEQ ID NO: 7; | 25686 |
| | region, HBV Ab | | WO2014048910; CA2884388; | |
| | | | CN104662041A; EP2900692 | |
| HEPBD19 | Heavy chain variable | | WO2013165972 SEQ ID NO: 45 | 25687 |
| | region, HBV Ab | | | |
| HEPBD20 | Heavy chain variable | | WO2013165972 SEQ ID NO: 54 | 25688 |
| | region, HBV Ab | | | |
| HEPBD21 | Heavy chain variable | | WO2013165972 SEQ ID NO: 63 | 25689 |
| | region, HBV Ab | | | |
| HEPBD22 | Heavy chain variable | | WO2013165972 SEQ ID NO: 72 | 25690 |
| | region, HBV Ab | | | |
| HEPBD23 | Heavy chain variable | | WO2013165972 SEQ ID NO: 81 | 25691 |
| | region, HBV Ab | | | |
| HEPBD24 | Heavy chain variable | | WO2013165972 SEQ ID NO: 90 | 25692 |
| | region, HBV Ab | | | |
| HEPBD25 | Heavy chain variable | | WO2013165972 SEQ ID NO: 99 | 25693 |
| | region, HBV Ab | | | |
| HEPBD26 | Heavy chain variable | | WO2013165972 SEQ ID NO: 108 | 25694 |
| | region, HBV Ab | | | |
| HEPBD27 | Heavy chain variable | | WO2013165972 SEQ ID NO: 117 | 25695 |
| | region, HBV Ab | | | |
| HEPBD28 | Heavy chain variable | | WO2013165972 SEQ ID NO: 126 | 25696 |
| | region, HBV Ab | | | |
| HEPBD29 | Heavy chain variable | | WO2013165972 SEQ ID NO: 135 | 25697 |
| | region, HBV Ab | | | |
| HEPBD30 | Heavy chain variable | | WO2013165972 SEQ ID NO: 144 | 25698 |
| | region, HBV Ab | | | |
| HEPBD31 | Heavy chain variable | | WO2013165972 SEQ ID NO: 153 | 25699 |
| | region, HBV Ab | | | |
| HEPBD32 | Heavy chain variable | | WO2013165972 SEQ ID NO: 162 | 25700 |
| | region, HBV Ab | | | |
| HEPBD33 | Heavy chain variable | | WO2013165972 SEQ ID NO: 171 | 25701 |
| | region, HBV Ab | | | |
| HEPBD34 | Heavy chain variable | | WO2013165972 SEQ ID NO: 180 | 25702 |
| | region, HBV Ab | | | |
| HEPBD35 | Heavy chain variable | | WO2013165972 SEQ ID NO: 189 | 25703 |
| | region, HBV Ab | | | |
| HEPBD36 | Heavy chain variable | | WO2013165972 SEQ ID NO: 198 | 25704 |
| | region, HBV Ab | | | |
| HEPBD37 | Heavy chain variable | | WO2013165972 SEQ ID NO: 207 | 25705 |
| | region, HBV Ab | | | |
| HEPBD38 | Heavy chain variable | | WO2013165972 SEQ ID NO: 405 | 25706 |
| | region, HBV Ab | | | |
| HEPBD39 | Heavy chain variable | | WO2013165972 SEQ ID NO: 409 | 25707 |
| | region, HBV Ab | | | |
| HEPBD40 | Heavy chain variable | | WO2013165972 SEQ ID NO: 412 | 25708 |
| | region, HBV Ab | | | |
| HEPBD41 | Heavy chain variable | | WO2013165972 SEQ ID NO: 418 | 25709 |
| | region, HBV Ab | | | |
| HEPBD42 | Heavy chain variable | | WO2013165972 SEQ ID NO: 421 | 25710 |
| | region, HBV Ab | | | |
| HEPBD43 | Heavy chain variable | | WO2009069916 SEQ ID NO: 1 | 25711 |
| | region, HBV Ab | | | |
| HEPBD44 | Heavy chain variable | PE1-1 | WO1994011495 | 25712 |
| | region, HBV Ab | | | |
| HEPBD45 | Heavy chain variable | ZM1-1 | WO1994011495 | 25713 |
| | region, HBV Ab | | | |
| HEPBD46 | Heavy chain variable | ZM1-2 | WO1994011495 | 25714 |
| | region, HBV Ab | | | |
| HEPBD47 | Heavy chain variable | MD3-4 | WO1994011495 | 25715 |
| | region, HBV Ab | | | |
| HEPBD48 | Heavy chain variable | A2E2 | CN102757492 SEQ ID NO: 2 | 25716 |
| | region, HBV Ab | | | |
| HEPBD49 | Heavy chain variable | C9G9 | CN102757492 SEQ ID NO: 6 | 25717 |
| | region, HBV Ab | | | |
| HEPBD50 | Heavy chain variable | | CN104530228 SEQ ID NO: 3 | 25718 |
| | region, HBV Ab | | | |
| HEPBD51 | Heavy chain variable | | CN104530228 SEQ ID NO: 4 | 25719 |
| | region, HBV Ab | | | |
| HEPBD52 | Heavy chain variable | Ab19 | U.S. Pat. No. 8,580,256 SEQ ID NO: 2 | 25720 |
| | region, HBV Ab | | | |
| HEPBD53 | Heavy chain variable | Ab17 | U.S. Pat. No. 8,580,256 SEQ ID NO: 4 | 25721 |
| | region, HBV Ab | | | |
| HEPBD54 | Heavy chain variable | KR127 | U.S. Pat. No. 8,420,353 SEQ ID NO: 28 | 25722 |
| | region, HBV Ab | | | |
| HEPBD55 | Heavy chain variable | DP7 | U.S. Pat. No. 8,420,353 SEQ ID NO: 32 | 25723 |
| | region, HBV Ab | | | |
| HEPBD56 | Heavy chain variable | HZI | U.S. Pat. No. 8,420,353 SEQ ID NO: 36 | 25724 |
| | region, HBV Ab | | | |
| HEPBD57 | Heavy chain variable | MBL-HCV1 | U.S. Pat. No. 8,551,484 SEQ ID NO: 1 | 25725 |
| | region, HCV Ab | (Antibody | | |
| | | produced by | | |
| | | clone 83-128) | | |
| HEPBD58 | Heavy chain variable | MBL-HCV1 | U.S. Pat. No. 8,551,484 SEQ ID NO: 3 | 25726 |
| | region, HCV Ab | (Antibody | | |
| | | produced by | | |
| | | clone 95-2) | | |
| HEPBD59 | Heavy chain variable | MBL-HCV1 | U.S. Pat. No. 8,551,484 SEQ ID NO: 5 | 25727 |
| | region, HCV Ab | (Antibody | | |
| | | produced by | | |
| | | clone 95-14) | | |
| HEPBD60 | Heavy chain variable | Clone 13 | U.S. Pat. No. 7,250,166 SEQ ID NO: 1 | 25728 |
| | region, HCV Ab | | | |
| HEPBD61 | Heavy chain variable | Clone 98 | U.S. Pat. No. 7,250,166 SEQ ID NO: 3 | 25729 |
| | region, HCV Ab | | | |
| HEPBD62 | Heavy chain variable | Clone 1:4 | U.S. Pat. No. 7,250,166 SEQ ID NO: 5 | 25730 |
| | region, HCV Ab | | | |
| HEPBD63 | Heavy chain variable | Clone 1:8 | U.S. Pat. No. 7,250,166 SEQ ID NO: 7 | 25731 |
| | region, HCV Ab | | | |
| HEPBD64 | Heavy chain variable | Clone 1:9 | U.S. Pat. No. 7,250,166 SEQ ID NO: 9 | 25732 |
| | region, HCV Ab | | | |
| HEPBD65 | Heavy chain variable | Clone 1:10 | U.S. Pat. No. 7,250,166 SEQ ID NO: 11 | 25733 |
| | region, HCV Ab | | | |
| HEPBD66 | Heavy chain variable | Clone 4:6 | U.S. Pat. No. 7,250,166 SEQ ID NO: 13 | 25734 |
| | region, HCV Ab | | | |
| HEPBD67 | Heavy chain variable | Clone 6a:5 | U.S. Pat. No. 7,250,166 SEQ ID NO: 15 | 25735 |
| | region, HCV Ab | | | |
| HEPBD68 | Heavy chain variable | Clone 2a:2 | U.S. Pat. No. 7,250,166 SEQ ID NO: 17 | 25736 |
| | region, HCV Ab | | | |
| HEPBD69 | Heavy chain variable | Clone 2a:4 | U.S. Pat. No. 7,250,166 SEQ ID NO: 19 | 25737 |
| | region, HCV Ab | | | |
| HEPBD70 | Heavy chain variable | Clone 2a:5 | U.S. Pat. No. 7,250,166 SEQ ID NO: 21 | 25738 |
| | region, HCV Ab | | | |
| HEPBD71 | Heavy chain variable | Clone 2a:13 | U.S. Pat. No. 7,250,166 SEQ ID NO: 23 | 25739 |
| | region, HCV Ab | | | |
| HEPBD72 | Heavy chain variable | Clone 2a:14 | U.S. Pat. No. 7,250,166 SEQ ID NO: 25 | 25740 |
| | region, HCV Ab | | | |
| HEPBD73 | Heavy chain variable | Clone 2a:23 | U.S. Pat. No. 7,250,166 SEQ ID NO: 27 | 25741 |
| | region, HCV Ab | | | |
| HEPBD74 | Heavy chain variable | Clone 2a:23 | U.S. Pat. No. 7,250,166 SEQ ID NO: 29 | 25742 |
| | region, HCV Ab | | | |
| HEPBD75 | Heavy chain variable | Clone 2a:25 | U.S. Pat. No. 7,250,166 SEQ ID NO: 31 | 25743 |
| | region, HCV Ab | | | |
| HEPBD76 | Heavy chain variable | Clone 2a:30 | U.S. Pat. No. 7,250,166 SEQ ID NO: 33 | 25744 |
| | region, HCV Ab | | | |
| HEPBD77 | Heavy chain variable | Clone 2a:32 | U.S. Pat. No. 7,250,166 SEQ ID NO: 35 | 25745 |
| | region, HCV Ab | | | |
| HEPBD78 | Heavy chain variable | Clone 2a:33 | U.S. Pat. No. 7,250,166 SEQ ID NO: 37 | 25746 |
| | region, HCV Ab | | | |
| HEPBD79 | Heavy chain variable | Clone 2a:37 | U.S. Pat. No. 7,250,166 SEQ ID NO: 39 | 25747 |
| | region, HCV Ab | | | |
| HEPBD80 | Heavy chain variable | Clone 2a:40 | U.S. Pat. No. 7,250,166 SEQ ID NO: 41 | 25748 |
| | region, HCV Ab | | | |
| HEPBD81 | Heavy chain variable | Clone 2b:1 | U.S. Pat. No. 7,250,166 SEQ ID NO: 43 | 25749 |
| | region, HCV Ab | | | |
| HEPBD82 | Heavy chain variable | Clone 2b:3 | U.S. Pat. No. 7,250,166 SEQ ID NO: 45 | 25750 |
| | region, HCV Ab | | | |
| HEPBD83 | Heavy chain variable | Clone 2b:4 | U.S. Pat. No. 7,250,166 SEQ ID NO: 47 | 25751 |
| | region, HCV Ab | | | |
| HEPBD84 | Heavy chain variable | Clone 2b:5 | U.S. Pat. No. 7,250,166 SEQ ID NO: 49 | 25752 |
| | region, HCV Ab | | | |
| HEPBD85 | Heavy chain variable | Clone 2b:7 | U.S. Pat. No. 7,250,166 SEQ ID NO: 51 | 25753 |
| | region, HCV Ab | | | |
| HEPBD86 | Heavy chain variable | Clone 2b:9 | U.S. Pat. No. 7,250,166 SEQ ID NO: 53 | 25754 |
| | region, HCV Ab | | | |
| HEPBD87 | Heavy chain variable | Clone 2b:10 | U.S. Pat. No. 7,250,166 SEQ ID NO: 55 | 25755 |
| | region, HCV Ab | | | |
| HEPHD88 | Heavy chain variable | anti-NS3 Fab | U.S. Pat. No. 7,314,919 SEQ ID NO: 1 | 25756 |
| | region, HCV Ab | | | |
| HEPBD89 | Heavy chain variable | Antibody | U.S. Pat. No. 8,551,484 SEQ ID NO: 32 | 25757 |
| | region, HCV Ab | produced by | | |
| | | clone 95-14 | | |
| HEPBD90 | Heavy chain variable | Antibody | U.S. Pat. No. 8,551,484 SEQ ID NO: 33 | 25758 |
| | region, HCV Ab | produced by | | |
| | | clone 95-38 | | |
| HEPBD91 | Heavy chain variable | Antibody | U.S. Pat. No. 8,551,484 SEQ ID NO: 34 | 25759 |
| | region, HCV Ab | produced by | | |
| | | clone 95-25 | | |
| HEPBD92 | Heavy chain variable | Antibody | U.S. Pat. No. 8,551,484 SEQ ID NO: 35 | 25760 |
| | region, HCV Ab | produced by | | |
| | | clone 95.42 | | |
| HEPBD93 | Heavy chain variable | Antibody | U.S. Pat. No. 8,551,484 SEQ ID NO: 36 | 25761 |
| | region, HCV Ab | produced by | | |
| | | clone 95-43 | | |
| HEPBD94 | Heavy chain variable | Antibody | U.S. Pat. No. 8,551,484 SEQ ID NO: 37 | 25762 |
| | region, HCV Ab | produced by | | |
| | | clone 95-49 | | |
| HEPBD95 | Heavy chain variable | Antibody | U.S. Pat. No. 8,551,484 SEQ ID NO: 38 | 25763 |
| | region, HCV Ab | produced by | | |
| | | clone 95-54 | | |
| HEPBD96 | Heavy chain variable | Antibody | U.S. Pat. No. 8,551,484 SEQ ID NO: 39 | 25764 |
| | region, HCV Ab | produced by | | |
| | | clone 95-58 | | |
| HEPBD97 | Heavy chain variable | Antibody | U.S. Pat. No. 8,551,484 SEQ ID NO: 40 | 25765 |
| | region, HCV Ab | produced by | | |
| | | clone 95-62 | | |
| HEPBD98 | Heavy chain variable | HC-84.1 | US20130084301 SEQ ID NO: 55 | 25766 |
| | region, HCV Ab | | | |
| HEPBD99 | Heavy chain variable | HC-84.20 | US20130084301 SEQ ID NO: 56 | 25767 |
| | region, HCV Ab | | | |
| HEPBD100 | Heavy chain variable | HC-84.21 | US20130084301 SEQ ID NO: 57 | 25768 |
| | region, HCV Ab | | | |
| HEPBD101 | Heavy chain variable | HC-84.22 | US20130084301 SEQ ID NO: 58 | 25769 |
| | region, HCV Ab | | | |
| HEPBD102 | Heavy chain variable | HC-23 | US20130084301 SEQ ID NO: 59 | 25770 |
| | region, HCV Ab | | | |
| HEPBD103 | Heavy chain variable | HC-84.24 | US20130084301 SEQ ID NO: 60 | 25771 |
| | region, HCV Ab | | | |
| HEPBD104 | Heavy chain variable | HC-84.25 | US20130084301 SEQ ID NO: 61 | 25772 |
| | region, HCV Ab | | | |
| HEPBD105 | Heavy chain variable | HC-84.26 | US20130084301 SEQ ID NO: 62 | 25773 |
| | region, HCV Ab | | | |
| HEPBD106 | Heavy chain variable | HC-84.27. | US20130084301 SEQ ID NO: 63 | 25774 |
| | region, HCV Ab | | | |
| HEPBD107 | Heavy chain variable | AOT3 | US20120009196 SEQ ID NO: 1 | 25775 |
| | region, HCV Ab | | | |
| HEPBD108 | Heavy chain variable | C11-3 | US20120009196 SEQ ID NO: 3 | 25776 |
| | region, HCV Ab | | | |
| HEPBD109 | Heavy chain variable | C11-7 | US20120009196 SEQ ID NO: 5 | 25777 |
| | region, HCV Ab | | | |
| HEPBD110 | Heavy chain variable | C11-9 | US20120009196 SEQ ID NO: 7 | 25778 |
| | region, HCV Ab | | | |
| HEPBD111 | Heavy chain variable | C11-14 | US20120009196 SEQ ID NO: 9 | 25779 |
| | region, HCV Ab | | | |
| HEPBD112 | Heavy chain variable | | WO2014065822 SEQ ID NO: 3 | 25780 |
| | region, HCV Ab | | | |
| HEPBD113 | Heavy chain variable | | WO2014065822 SEQ ID NO: 7 | 25781 |
| | region, HCV Ab | | | |
| HEPBD114 | Heavy chain variable | mPA-29 | WO2007143701 SEQ ID NO: 2 | 25782 |
| | region, HCV Ab | | | |
| HEPBD115 | Heavy chain variable | Hc33.1 | Li Y. et al., Structural basis for penetration | 25783 |
| | region, HCV Ab | | of the glycan shield of hepatitis C virUSe2 | |
| | | | glycoprotein by a broadly neutralizing | |
| | | | human antibody; J. Biol. Chem. 290 (16), | |
| | | | 10117-10125 (2015) NCBI Accession # | |
| | | | 4XVJ_H (141aa) | |
| HEPBD116 | Heavy chain variable | | Martin, F., et al., Affinity selection of a | 25784 |
| | region, HCV Ab | | camelized V(H) domain antibody inhibitor | |
| | | | of hepatitis C virUSNS3 protease; Protein | |
| | | | Eng. 10 (5), 607-614 (1997NCBI | |
| | | | Accession # 1OL0_B (121aa) | |
| HEPBD117 | Heavy chain variable | | US20150118242 SEQ ID NO: 2 | 25785 |
| | region, HCV Ab | | | |
| HEPBD118 | Heavy chain variable | | US20150166637 SEQ ID NO: 1, | 25786 |
| | region, Human HBV | | WO2014010890; CA2878155, | |
| | antibody that binds to | | CN104487090, EP2858674 | |
| | the surface antigen | | | |
| | (HBsAg) | | | |
| HEPBD119 | Heavy chain variable | | US20150166637 SEQ ID NO: 2; | 25787 |
| | region, Human HBV | | WO2014010890; CA2878155, | |
| | antibody that binds to | | CN104487090, EP2858674 | |
| | the surface antigen | | | |
| | (HBsAg) | | | |
| HEPBD120 | Heavy chain variable | | US20150166637 SEQ ID NO: 3; | 25788 |
| | region, Human HBV | | WO2014010890; CA2878155, | |
| | antibody that binds to | | CN104487090, EP2858674 | |
| | the surface antigen | | | |
| | (HBsAg) | | | |
| HEPBD121 | Heavy chain variable | | US20150166637 SEQ ID NO: 4; | 25789 |
| | region, Human HBV | | WO2014010890; CA2878155, | |
| | antibody that binds to | | CN104487090, EP2858674 | |
| | the surface antigen | | | |
| | (HBsAg) | | | |
| HEPBD122 | Heavy chain variable | | US20150166637 SEQ ID NO: 5; | 25790 |
| | region, Human HBV | | WO2014010890; CA2878155, | |
| | antibody that binds to | | CN104487090, EP2858674 | |
| | the surface antigen | | | |
| | (HBsAg) | | | |
| HEPBD123 | Heavy chain variable | c18/A2 | US20120308580 SEQ ID NO: 2; | 25791 |
| | region, Monoclonal | | WO2011062562; CN102781961, | |
| | HBV antibody | | EP2501723 | |
| HEPBD124 | Heavy chain variable | | US20110097270 SEQ ID NO: 1 | 25792 |
| | region, Neutralizing | | | |
| | monoclonal HBV | | | |
| | antibody | | | |
| HEPBD125 | Heavy chain, full | | US20150232537 SEQ ID NO: 9; | 25793 |
| | HBV Ab | | WO2014048910; CA2884388; | |
| | | | CN104662041A; EP2900692 | |
| HEPBD126 | Heavy chain, HBV Ab | HBFab21 | CN103588874 SEQ ID NO: 2 | 25794 |
| HEPBD127 | Heavy chain, HCV Ab | Fab clone 1:5 | U.S. Pat. No. 6,747,136 SEQ ID NO: 1 | 25795 |
| HEPBD128 | Heavy chain, HCV Ab | Fab clone 1:7 | U.S. Pat. No. 6,747,136 SEQ ID NO: 2 | 25796 |
| HEPBD129 | Heavy chain, HCV Ab | Fab clone 1:11 | U.S. Pat. No. 6,747,136 SEQ ID NO: 3 | 25797 |
| HEPBD130 | Heavy chain, HCV Ab | Fab clone L3 | U.S. Pat. No. 6,747,136 SEQ ID NO: 4 | 25798 |
| HEPBD131 | Heavy chain, HCV Ab | Fab clone L1 | U.S. Pat. No. 6,747,136 SEQ ID NO: 5 | 25799 |
| HEPBD132 | Heavy chain, HCV Ab | Fab clone A8 | U.S. Pat. No. 6,747,136 SEQ ID NO: 6 | 25800 |
| HEPBD133 | Heavy chain, HCV Ab | Fab clone A12 | U.S. Pat. No. 6,747,136 SEQ ID NO: 7 | 25801 |
| HEPBD134 | Heavy chain, HCV Ab | HCV-AB 68 | U.S. Pat. No. 7,241,445 SEQ ID NO: 3 | 25802 |
| HEPBD135 | Heavy chain, HCV Ab | e8 | U.S. Pat. No. 7,727,529 SEQ ID NO: 1 | 25803 |
| HEPBD136 | Heavy chain, HCV Ab | e10 | U.S. Pat. No. 7,727,529 SEQ ID NO: 3 | 25804 |
| HEPBD137 | Heavy chain, HCV Ab | e20 | U.S. Pat. No. 7,727,529 SEQ ID NO: 5 | 25805 |
| HEPBD138 | Heavy chain, HCV Ab | e137 | U.S. Pat. No. 7,727,529 SEQ ID NO: 7 | 25806 |
| HEPBD139 | Heavy chain, HCV Ab | e301 | U.S. Pat. No. 7,727,529 SEQ ID NO: 9 | 25807 |
| HEPBD140 | Heavy chain, HCV Ab | e509 | U.S. Pat. No. 7,727,529 SEQ ID NO: 11 | 25808 |
| HEPBD141 | Heavy chain, HCV Ab | 5D2 | US20090104207 SEQ ID NO: 7 | 25809 |
| HEPBD142 | Heavy chain, HCV Ab | Mab V | WO2013186752 SEQ ID NO: 3 | 25810 |
| HEPBD143 | Heavy chain, HCV Ab | Mab VI | WO2013186752 SEQ ID NO: 5 | 25811 |
| HEPBD144 | Heavy chain, HCV Ab | | WO2007143701 SEQ ID NO: 12 | 25812 |
| HEPBD145 | Heavy chain, HCV Ab | HuPA29VH#1 | WO2007143701 SEQ ID NO: 15 | 25813 |
| HEPBD146 | Heavy chain, HCV Ab | HuPA29VH#2 | WO2007143701 SEQ ID NO: 16 | 25814 |
| HEPBD147 | Heavy chain, HCV Ab | HuPA29VH#3 | WO2007143701 SEQ ID NO: 17 | 25815 |
| HEPBD148 | Heavy chain, HCV Ab | PA29 | WO2007143701 SEQ ID NO: 28 | 25816 |
| HEPBD149 | Heavy chain, HCV Ab | Ap33 | Kong, L., et al., Structure of Hepatitis C | 25817 |
| | | | Virus Envelope Glycoprotein E2 Antigenic | |
| | | | Site 412 to 423 in Complex with Antibody | |
| | | | AP33; J. Virol. 86 (23), 13085-13088 | |
| | | | (2012) NCBI Accession # 4G6A _H | |
| | | | (224aa) | |
| HEPBD150 | Heavy chain, HCV Ab | Single Chain Fv | Gilmartin, A. A., et al., Protein Eng. Des. | 25818 |
| | | Fragment 1:7 | Sel. 25 (2), 59-66 (2012) NCBI Accession | |
| | | | # 3U6R_A(149aa) | |
| HEPBD151 | Heavy Chain, HCV | Ar3c | Kong, L., et al., Hepatitis C virUSE2 | 25819 |
| | Fab | | envelope glycoprotein core structure; | |
| | | | Science 342 (6162), 1090-1094 (2013) | |
| | | | NCBI Accession # 4MWF_A (233aa) | |
| HEPBD152 | Heavy Chain, HCV | Mrct10.v362 | Pantua, H., et al., Glycan shifting on | 25820 |
| | Fab | | hepatitis C virus (HCV) e2 glycoprotein is | |
| | | | a mechanism for escape from broadly | |
| | | | neutralizing antibodies; J. Mol. Biol. 425 | |
| | | | (11), 1899-1914 (2013) NCBI Accession # | |
| | | | HS6_H (226aa) | |
| HEPBD153 | Heavy Chain, Hcv1 | Hcv1, P2(1) | Kong, L., et al., Structural basis of | 25821 |
| | HCV Ab | Form | hepatitis C virus neutralization by broadly | |
| | | | neutralizing antibody HCV1; Proc. Natl. | |
| | | | Acad. Sci. U.S.A. 109 (24), 9499-9504 | |
| | | | (2012) NCBI Accession # 4DGV_H | |
| | | | (226aa) | |
| HEPBD154 | Heavy Chain, Hcv1 | Hcv1, C2 Form | Kong, L., et al., Structural basis of | 25822 |
| | HCV Ab | | hepatitis C virus neutralization by broadly | |
| | | | neutralizing antibody HCV1; Proc. Natl. | |
| | | | Acad. Sci. U.S.A. 109 (24), 9499-9504 | |
| | | | (2012) NCBI Accession # 4DGY_H | |
| | | | (226aa) | |
| HEPBD155 | Heavy gamma chain | P18-9E | U.S. Pat. No. 8,592,559 SEQ ID NO: 13 | 25823 |
| | variable, HCV Ab | | | |
| HEPBD156 | Heavy-chain-only, | VHH D03 | WO2014053634 SEQ ID NO: 4 | 25824 |
| | HCV Ab | | | |
| HEPBD157 | Heavy-chain-only, | VHH C09 | WO2014053634 SEQ ID NO: 5 | 25825 |
| | HCV Ab | | | |
| HEPBD158 | Heavy-chain-only, | Bl 1 | WO2014053634 SEQ ID NO: 6 | 25826 |
| | HCV Ab | | | |
| HEPBD159 | Heavy-chain-only, | D04 | WO2014053634 SEQ ID NO: 7 | 25827 |
| | HCV Ab | | | |
| HEPBD160 | Light chain full, HBV | | US20150232537 SEQ ID NO: 10; | 25828 |
| | Ab | | WO2014048910; CA2884388; | |
| | | | CN104662041A; EP2900692 | |
| HEPBD161 | Light chain kappa, | | Esposito, C., et al., Recombinant human | 25829 |
| | partial, HCV Ab | | antibodies specific for hepatitis C virus | |
| | | | proteins; Arch. Virol. 142 (3), 601-610 | |
| | | | (1997) NCBI Accession # | |
| | | | CAA54913.1(110aa) | |
| HEPBD162 | Light chain variable | c18/A2 | US20120308580 SEQ ID NO: 1; | 25830 |
| | domain, monoclonal | | WO2011062562; CN102781961, EP2501723 | |
| | HBV antibody | | | |
| HEPBD163 | Light chain variable | | US20110097270 SEQ ID NO: 9 | 25831 |
| | domain, neutralizing | | | |
| | monoclonal HBV | | | |
| | antibody, | | | |
| HEPBD164 | Light chain variable | | EP0521348 | 25832 |
| | gene, Chimeric HBV | | | |
| | Ab | | | |
| HEPBD165 | Light chain variable | E183/A2 | US20120308580 SEQ ID NO: 32; | 25833 |
| | region, HBV Ab | | WO2011062562; CN102781961, EP2501723 | |
| HEPBD166 | Light chain variable | HB48-33 | U.S. Pat. No. 8,840,895 SEQ ID NO: 2 | 25834 |
| | region, HBV Ab | | | |
| HEPBD167 | Light chain variable | HB48-35 | U.S. Pat. No. 8,840,895 SEQ ID NO: 3 | 25835 |
| | region, HBV Ab | | | |
| HEPBD168 | Light chain variable | HB48-59 | U.S. Pat. No. 8,840,895 SEQ ID NO: 4 | 25836 |
| | region, HBV Ab | | | |
| HEPBD169 | Light chain variable | LFW22-31 | U.S. Pat. No. 7,435,414 SEQ ID NO: 36; | 25837 |
| | region, HBV Ab | | US20060014937; WO2005100400; | |
| | | | CN1980956 | |
| HEPBD170 | Light chain variable | LFW22-312 | U.S. Pat. No. 7,435,414 SEQ ID NO: 37; | 25838 |
| | region, HBV Ab | | US20060014937; WO2005100400; | |
| | | | CN1980956 | |
| HEPBD171 | Light chain variable | | WO2013165972 SEQ ID NO: 216 | 25839 |
| | region, HBV Ab | | | |
| HEPBD172 | Light chain variable | | WO2013165972 SEQ ID NO: 225 | 25840 |
| | region, HBV Ab | | | |
| HEPBD173 | Light chain variable | | WO2013165972 SEQ ID NO: 234 | 25841 |
| | region, HBV Ab | | | |
| HEPBD174 | Light chain variable | | WO2013165972 SEQ ID NO: 243 | 25842 |
| | region, HBV Ab | | | |
| HEPBD175 | Light chain variable | | WO2013165972 SEQ ID NO: 252 | 25843 |
| | region, HBV Ab | | | |
| HEPBD176 | Light chain variable | | WO2013165972 SEQ ID NO: 261 | 25844 |
| | region, HBV Ab | | | |
| HEPBD177 | Light chain variable | | WO2013165972 SEQ ID NO: 270 | 25845 |
| | region, HBV Ab | | | |
| HEPBD178 | Light chain variable | | WO2013165972 SEQ ID NO: 279 | 25846 |
| | region, HBV Ab | | | |
| HEPBD179 | Light chain variable | | WO2013165972 SEQ ID NO: 288 | 25847 |
| | region, HBV Ab | | | |
| HEPBD180 | Light chain variable | | WO2013165972 SEQ ID NO: 297 | 25848 |
| | region, HBV Ab | | | |
| HEPBD181 | Light chain variable | | WO2013165972 SEQ ID NO: 306 | 25849 |
| | region, HBV Ab | | | |
| HEPBD182 | Light chain variable | | WO2013165972 SEQ ID NO: 315 | 25850 |
| | region, HBV Ab | | | |
| HEPBD183 | Light chain variable | | WO2013165972 SEQ ID NO: 324 | 25851 |
| | region, HBV Ab | | | |
| HEPBD184 | Light chain variable | | WO2013165972 SEQ ID NO: 333 | 25852 |
| | region, HBV Ab | | | |
| HEPBD185 | Light chain variable | | WO2013165972 SEQ ID NO: 342 and 351 | 25853 |
| | region, HBV Ab | | | |
| HEPBD186 | Light chain variable | | WO2013165972 SEQ ID NO: 360 | 25854 |
| | region, HBV Ab | | | |
| HEPBD187 | Light chain variable | | WO2013165972 SEQ ID NO: 369 | 25855 |
| | region, HBV Ab | | | |
| HEPBD188 | Light chain variable | | WO2013165972 SEQ ID NO: 378 | 25856 |
| | region, HBV Ab | | | |
| HEPBD189 | Light chain variable | | WO2013165972 SEQ ID NO: 387 | 25857 |
| | region, HBV Ab | | | |
| HEPBD190 | Light chain variable | | WO2013165972 SEQ ID NO: 396 | 25858 |
| | region, HBV Ab | | | |
| HEPBD191 | Light chain variable | | WO2009069916 SEQ ID NO: 2 | 25859 |
| | region, HBV Ab | | | |
| HEPBD192 | Light chain variable | PE1-1 | WO1994011495 | 25860 |
| | region, HBV Ab | | | |
| HEPBD193 | Light chain variable | ZM1-1 | WO1994011495 | 25861 |
| | region, HBV Ab | | | |
| HEPBD194 | Light chain variable | ZM1-2 | WO1994011495 | 25862 |
| | region, HB V Ab | | | |
| HEPBD195 | Light chain variable | MD3-4 | WO1994011495 | 25863 |
| | region, HBV Ab | | | |
| HEPBD196 | Light chain variable | A2E2 | CN102757492 SEQ ID NO: 4 | 25864 |
| | region, HBV Ab | | | |
| HEPBD197 | Light chain variable | C9G9 | CN102757492 SEQ ID NO: 8 | 25865 |
| | region, HBV Ab | | | |
| HEPBD198 | Light chain variable | | CN104530228 SEQ ID NO: 1 | 25866 |
| | region, HBV Ab | | | |
| HEPBD199 | Light chain variable | | CN104530228 SEQ ID NO: 2 | 25867 |
| | region, HBV Ab | | | |
| HEPBD200 | Light chain variable | Ab19 | U.S. Pat. No. 8,580,256 SEQ ID NO: 1 | 25868 |
| | region, HBV Ab | | | |
| HEPBD201 | Light chain variable | Ab17 | U.S. Pat. No. 8,580,256 SEQ ID NO: 3 | 25869 |
| | region, HBV Ab | | | |
| HEPBD202 | Light chain variable | KR127 | U.S. Pat. No. 8,420,353 SEQ ID NO: 4 | 25870 |
| | region, HBV Ab | | | |
| HEPBD203 | Light chain variable | KR127 | U.S. Pat. No. 8,420,353 SEQ ID NO: 2 | 25871 |
| | region, HBV Ab | | | |
| HEPBD204 | Light chain variable | KR127 | U.S. Pat. No. 8,420,353 SEQ ID NO: 30 | 25872 |
| | region, HBV Ab | | | |
| HEPBD205 | Light chain variable | DPK12 | U.S. Pat. No. 8,420,353 SEQ ID NO: 34 | 25873 |
| | region, HBV Ab | | | |
| HEPBD206 | Light chain variable | HZI | U.S. Pat. No. 8,420,353 SEQ ID NO: 38 | 25874 |
| | region, HBV Ab | | | |
| HEPBD207 | Light chain variable | | U.S. Pat. No. 7,112,664 SEQ ID NO: 7; U.S. Pat. No. 6,680,053, | 25875 |
| | region, HBV Ab | | U.S. Pat. No. 6,924,368, US20020061581, | |
| | | | US20040191259, US20050249753, | |
| | | | WO2001092529 | |
| HEPBD208 | Light chain variable | Ab17.1.4 1 | USRE39586 SEQ ID NO: 2; U.S. Pat. No. 6,146,629; | 25876 |
| | region, HBV Ab | | WO1997047653 | |
| HEPBD209 | Light chain variable | Ab 19.79.5 | USRE40831 SEQ ID NO: 2 | 25877 |
| | region, HBV Ab | | | |
| HEPBD210 | Light chain variable | | US20150232537 SEQ ID NO: 8; | 25878 |
| | region, HBV Ab | | WO2014048910; CA2884388; | |
| | | | CN104662041A; EP2900692 | |
| HEPBD211 | Light chain variable | | US20100260712 SEQ ID NO: 2; | 25879 |
| | region, HBV Ab | | WO2009069917 | |
| HEPBD212 | Light chain variable | | WO2015107126 SEQ ID NO: 4 | 25880 |
| | region, HBV Ab | | | |
| HEPBD213 | Light chain variable | MBL-HCV1 | U.S. Pat. No. 8,551,484 SEQ ID NO: 2 | 25881 |
| | region, HCV Ab | (Antibody | | |
| | | produced by | | |
| | | clone 83-128) | | |
| HEPBD214 | Light chain variable | MBL-HCV1 | U.S. Pat. No. 8,551,484 SEQ ID NO: 4 | 25882 |
| | region, HCV Ab | (Antibody | | |
| | | produced by | | |
| | | clone 95-2) | | |
| HEPBD215 | Light chain variable | MBL-HV1 | U.S. Pat. No. 8,551,484 SEQ ID NO: 6 | 25883 |
| | region, HCV Ab | (Antibody | | |
| | | produced by | | |
| | | clone 073-1) | | |
| HEPBD216 | Light chain variable | Clone 13 | U.S. Pat. No. 7,250,166 SEQ ID NO: 2 | 25884 |
| | region, HCV Ab | | | |
| HEPBD217 | Light chain variable | Clone 98 | U.S. Pat. No. 7,250,166 SEQ ID NO: 4 | 25885 |
| | region, HCV Ab | | | |
| HEPBD218 | Light chain variable | Clone 1:4 | U.S. Pat. No. 7,250,166 SEQ ID NO: 6 | 25886 |
| | region, HCV Ab | | | |
| HEPBD219 | Light chain variable | Clone 1:8 | U.S. Pat. No. 7,250,166 SEQ ID NO: 8 | 25887 |
| | region, HCV Ab | | | |
| HEPBD220 | Light chain variable | Clone 1:9 | U.S. Pat. No. 7,250,166 SEQ ID NO: 10 | 25888 |
| | region, HCV Ab | | | |
| HEPBD221 | Light chain variable | Clone 1:10 | U.S. Pat. No. 7,250,166 SEQ ID NO: 12 | 25889 |
| | region, HCV Ab | | | |
| HEPBD222 | Light chain variable | Clone 4:6 | U.S. Pat. No. 7,250,166 SEQ ID NO: 14 | 25890 |
| | region, HCV Ab | | | |
| HEPBD223 | Light chain variable | Clone 6a:5 | U.S. Pat. No. 7,250,166 SEQ ID NO: 16 | 25891 |
| | region, HCV Ab | | | |
| HEPBD224 | Light chain variable | Clone 2a:2 | U.S. Pat. No. 7,250,166 SEQ ID NO: 18 | 25892 |
| | region, HCV Ab | | | |
| HEPBD225 | Light chain variable | Clone 2a:4 | U.S. Pat. No. 7,250,166 SEQ ID NO: 20 | 25893 |
| | region, HCV Ab | | | |
| HEPBD226 | Light chain variable | Clone 2a:5 | U.S. Pat. No. 7,250,166 SEQ ID NO: 22 | 25894 |
| | region, HCV Ab | | | |
| HEPBD227 | Light chain variable | Clone 2a:13 | U.S. Pat. No. 7,250,166 SEQ ID NO: 24 | 25895 |
| | region, HCV Ab | | | |
| HEPBD228 | Light chain variable | Clone 2a:14 | U.S. Pat. No. 7,250,166 SEQ ID NO: 26 | 25896 |
| | region, HCV Ab | | | |
| HEPBD229 | Light chain variable | Clone 2a:23 | U.S. Pat. No. 7,250,166 SEQ ID NO: 28 | 25897 |
| | region, HCV Ab | | | |
| HEPBD230 | Light chain variable | Clone 2a:23 | U.S. Pat. No. 7,250,166 SEQ ID NO: 30 | 25898 |
| | region, HCV Ab | | | |
| HEPBD231 | Light chain variable | Clone 2a:25 | U.S. Pat. No. 7,250,166 SEQ ID NO: 32 | 25899 |
| | region, HCV Ab | | | |
| HEPBD232 | Light chain variable | Clone 2a:30 | U.S. Pat. No. 7,250,166 SEQ ID NO: 34 | 25900 |
| | region, HCV Ab | | | |
| HEPBD233 | Light chain variable | Clone 2a:32 | U.S. Pat. No. 7,250,166 SEQ ID NO: 36 | 25901 |
| | region, HCV Ab | | | |
| HEPBD234 | Light chain variable | Clone 2a:33 | U.S. Pat. No. 7,250,166 SEQ ID NO: 38 | 25902 |
| | region, HCV Ab | | | |
| HEPBD235 | light chain variable | Clone 2a:37 | U.S. Pat. No. 7,250,166 SEQ ID NO: 40 | 25903 |
| | region, HCV Ab | | | |
| HEPBD236 | Light chain variable | Clone 2a:40 | U.S. Pat. No. 7,250,166 SEQ ID NO: 42 | 25904 |
| | region, HCV Ab | | | |
| HEPBD237 | Light chain variable | Clone 2b:1 | U.S. Pat. No. 7,250,166 SEQ ID NO: 44 | 25905 |
| | region, HCV Ab | | | |
| HEPBD238 | Light chain variable | Clone 2b:3 | U.S. Pat. No. 7,250,166 SEQ ID NO: 46 | 25906 |
| | region, HCV Ab | | | |
| HEPBD239 | Light chain variable | Clone 2b:4 | U.S. Pat. No. 7,250,166 SEQ ID NO: 48 | 25907 |
| | region, HCV Ab | | | |
| HEPBD240 | Light chain variable | Clone 2b:5 | U.S. Pat. No. 7,250,166 SEQ ID NO: 50 | 25908 |
| | region, HCV Ab | | | |
| HEPBD241 | Light chain variable | Clone 2b:7 | U.S. Pat. No. 7,250,166 SEQ ID NO: 52 | 25909 |
| | region, HCV Ab | | | |
| HEPBD242 | Light chain variable | Clone 2b:9 | U.S. Pat. No. 7,250,166 SEQ ID NO: 54 | 25910 |
| | region, HCV Ab | | | |
| HEPBD243 | Light chain variable | Clone 2b:10 | U.S. Pat. No. 7,250,166 SEQ ID NO: 56 | 25911 |
| | region, HCV Ab | | | |
| HEPBD244 | Light chain variable | anti-NS3 Fab | U.S. Pat. No. 7,314,919 SEQ ID NO: 6 | 25912 |
| | region, HCV Ab | | | |
| HEPBD245 | Light chain variable | | U.S. Pat. No. 7,507,408 SEQ ID NO: 2 | 25913 |
| | region, HCV Ab | | | |
| HEPBD246 | Light chain variable | | U.S. Pat. No. 7,507,408 SEQ ID NO: 4 | 25914 |
| | region, HCV Ab | | | |
| HEPBD247 | Light chain variable | | U.S. Pat. No. 7,507,408 SEQ ID NO: 6 | 25915 |
| | region, HCV Ab | | | |
| HEPBD248 | Light chain variable | Antibody | U.S. Pat. No. 8,551,484 SEQ ID NO: 44 | 25916 |
| | region, HCV Ab | produced by | | |
| | | clone 95-14 | | |
| HEPBD249 | Light chain variable | Antibody | U.S. Pat. No. 8,551,484 SEQ ID NO: 53 | 25917 |
| | region, HCV Ab | produced by | | |
| | | clone 95-38 | | |
| HEPBD250 | Light chain variable | HC-84.1 | US20130084301 SEQ ID NO: 64 | 25918 |
| | region, HCV Ab | | | |
| HEPBD251 | Light chain variable | HC-84.20 | US20130084301 SEQ ID NO: 65 | 25919 |
| | region, HCV Ab | | | |
| HEPBD252 | Light chain variable | HC-84.21 | US20130084301 SEQ ID NO: 66 | 25920 |
| | region, HCV Ab | | | |
| HEPBD253 | Light chain variable | HC-84.22 | US20130084301 SEQ ID NO: 67 | 25921 |
| | region, HCV Ab | | | |
| HEPBD254 | Light chain variable | HC-23 | US20130084301 SEQ ID NO: 68 | 25922 |
| | region, HCV Ab | | | |
| HEPBD255 | Light chain variable | HC-84.24 | US20130084301 SEQ ID NO: 69 | 25923 |
| | region, HCV Ab | | | |
| HEPBD256 | Light chain variable | HC-84.25 | US20130084301 SEQ ID NO: 70 | 25924 |
| | region, HCV Ab | | | |
| HEPBD257 | Light chain variable | HC-84.26 | US20130084301 SEQ ID NO: 71 | 25925 |
| | region, HCV Ab | | | |
| HEPBD258 | Light chain variable | HC-84.27. | US20130084301 SEQ ID NO. 72 | 25926 |
| | region, HCV Ab | | | |
| HEPBD259 | Light chain variable | AOT3 | US20120009196 SEQ ID NO: 2 | 25927 |
| | region, HCV Ab | | | |
| HEPBD260 | Light chain variable | C11-3 | US20120009196 SEQ ID NO: 4 | 25928 |
| | region, HCV Ab | | | |
| HEPBD261 | Light chain variable | C11-7 | US20120009196 SEQ ID NO: 6 | 25929 |
| | region, HCV Ab | | | |
| HEPBD262 | Light chain variable | C11-9 | US20120009196 SEQ ID NO: 8 | 25930 |
| | region, HCV Ab | | | |
| HEPBD263 | Light chain variable | C11-14 | US20120009196 SEQ ID NO: 10 | 25931 |
| | region, HCV Ab | | | |
| HEPBD264 | Light chain variable | | WO2014065822 SEQ ID NO: 5 | 25932 |
| | region, HCV Ab | | | |
| HEPBD265 | Light chain variable | | WO2014065822 SEQ ID NO: 9 | 25933 |
| | region, HCV Ab | | | |
| HEPBD266 | Light chain variable | Antibody light | WO2007143701 SEQ ID NO: 1 | 25934 |
| | region, HCV Ab | chain from | | |
| | | WO2007143701 | | |
| HEPBD267 | Light chain variable | Hc33.1 | Li Y. et al., Structural basis for penetration | 25935 |
| | region, HCV Ab | | of the glycan shield of hepatitis C virUSe2 | |
| | | | glycoprotein by a broadly neutralizing | |
| | | | human antibody; J. Biol. Chem. 290 (16), | |
| | | | 10117-10125 (2015) NCBI Accession # | |
| | | | 4XVJ_L (115aa) | |
| HEPBD268 | Light chain variable | | US20150118242 SEQ ID NO: 3 | 25936 |
| | region, HCV Ab | | | |
| HEPBD269 | Light chain variable | | US20150166637 SEQ ID NO: 6; | 25937 |
| | region, Human HBV | | WO2014010890; CA2878155, | |
| | antibody that binds to | | CN104487090; EP2858674 | |
| | the surface antigen | | | |
| | (HBsAg) | | | |
| HEPBD270 | Light chain variable | | US20150166637 SEQ ID NO: 7; | 25938 |
| | region, Human HBV | | WO2014010890; CA2878155, | |
| | antibody that binds to | | CN104487090, EP2858674 | |
| | the surface antigen | | | |
| | (HBsAg) | | | |
| HEPBD271 | Light chain variable | | US20150166637 SEQ ID NO: 8; | 25939 |
| | region, Human HBV | | WO2014010890; CA2878155, | |
| | antibody that binds to | | CN104487090, EP2858674 | |
| | the surface antigen | | | |
| | (HBsAg) | | | |
| HEPBD272 | Light chain variable | | US20150166637 SEQ ID NO: 9; | 25940 |
| | region, Human HBV | | WO2014010890; CA2878155, | |
| | antibody that binds to | | CN104487090, EP2858674 | |
| | the surface antigen | | | |
| | (HBsAg) | | | |
| HEPBD273 | Light chain variable | | US20150166637 SEQ ID NO: 10; | 25941 |
| | region, Human HBV | | WO2014010890, CA2878155, | |
| | antibody that binds to | | CN104487090, EP2858674 | |
| | the surface antigen | | | |
| | (HBsAg) | | | |
| HEPBD274 | Light chain variable | | Keck, Z. Y., et al., Human monoclonal | 25942 |
| | region, partial, HCV | | antibody to hepatitis C virUSE1 | |
| | Ab | | glycoprotein that blocks virus attachment | |
| | | | and viral infectivity; J. Virol. 78(13), | |
| | | | 7257-7263 (2004) NCBI Accession # | |
| | | | AAS47840 (147aa) | |
| HEPBD275 | Light chain, HCV Ab | Hu5b3.v3 | Pantua, H., et al., Glycan shifting on | 25943 |
| | | | hepatitis C virus (HCV) e2 glycoprotein is | |
| | | | a mechanism for escape from broadly | |
| | | | neutralizing antibodies; J. Mol. Biol. 425 | |
| | | | (11), 1899-1914 (2013) NCBI Accession # | |
| | | | 4HS8_L (218aa) | |
| HEPBD276 | Light chain, HCV Ab | Ap33 | Kong, L., et al., Structure of Hepatitis C | 25944 |
| | | | Virus Envelope Glycoprotein E2 Antigenic | |
| | | | Site 412 to 423 in Complex with Antibody | |
| | | | AP33; J. Virol. 86 (23), 13085-13088 | |
| | | | (2012) NCBI Accession # 4G6A_L | |
| | | | (218aa) | |
| HEPBD277 | Light chain, HBV Ab | HBFab21 | CN103588874 SEQ ID NO: 1 | 25945 |
| HEPBD278 | Light chain, HCV Ab | Fab clone 1:5 | U.S. Pat. No. 6,747,136 SEQ ID NO: 8 | 25946 |
| HEPBD279 | Light chain, HCV Ab | Fab clone 1:7 | U.S. Pat. No. 6,747,136 SEQ ID NO: 9 | 25947 |
| HEPBD280 | Light chain, HCV Ab | Fab clone 1:11 | U.S. Pat. No. 6,747,136 SEQ ID NO: 10 | 25948 |
| HEPBD281 | Light chain, HCV Ab | Fab clone L3 | U.S. Pat. No. 6,747,136 SEQ ID NO: 11 | 25949 |
| HEPBD282 | Light chain, HCV Ab | Fab clone L1 | U.S. Pat. No. 6,747,136 SEQ ID NO: 12 | 25950 |
| HEPBD283 | Light chain, HCV Ab | Fab clone A8 | U.S. Pat. No. 6,747,136 SEQ ID NO: 13 | 25951 |
| HEPBD284 | Light chain, HCV Ab | Fab clone A12 | U.S. Pat. No. 6,747,136 SEQ ID NO: 14 | 25952 |
| HEPBD285 | Light chain, HCV Ab | HCV#1 | U.S. Pat. No. 6,924,362 SEQ ID NO: 1 | 25953 |
| HEPBD286 | Light chain, HCV Ab | HCV#4 | U.S. Pat. No. 6,924,362 SEQ ID NO: 2 | 25954 |
| HEPBD287 | Light chain, HCV Ab | HCV#7 | U.S. Pat. No. 6,924,362 SEQ ID NO: 3 | 25955 |
| HEPBD288 | Light chain, HCV Ab | HCV#12 | U.S. Pat. No. 6,924,362 SEQ ID NO: 4 | 25956 |
| HEPBD289 | Light chain, HCV Ab | HGV#13 | U.S. Pat. No. 6,924,362 SEQ ID NO: 5 | 25957 |
| HEPBD290 | Light chain, HCV Ab | HCV-AB 68 | U.S. Pat. No. 7,241,445 SEQ ID NO: 4 | 25958 |
| HEPBD291 | Light chain, HCV Ab | e8 | U.S. Pat. No. 7,727,529 SEQ ID NO: 2 | 25959 |
| HEPBD292 | Light chain, HCV Ab | e10 | U.S. Pat. No. 7,727,529 SEQ ID NO: 4 | 25960 |
| HEPBD293 | Light chain, HCV Ab | e20 | U.S. Pat. No. 7,727,529 SEQ ID NO: 6 | 25961 |
| HEPBD294 | Light chain, HCV Ab | e137 | U.S. Pat. No. 7,727,529 SEQ ID NO: 8 | 25962 |
| HEPBD295 | Light chain, HCV Ab | e301 | U.S. Pat. No. 7,727,529 SEQ ID NO: 10 | 25963 |
| HEPBD296 | Light chain, HCV Ab | e509 | U.S. Pat. No. 7,727,529 SEQ ID NO: 12 | 25964 |
| HEPBD297 | Light chain, HCV Ab | 5D2 | US20090104207 SEQ ID NO: 8 | 25965 |
| HEPBD298 | Light chain, HCV Ab | MabV | WO2013186752 SEQ ID NO: 4 | 25966 |
| HEPBD299 | Light chain, HCV Ab | Mab VI | WO2013186752 SEQ ID NO: 6 | 25967 |
| HEPBD300 | Light chain, HCV Ab | | WO2007143701 SEQ ID NO: 11 | 25968 |
| HEPBD301 | Light chain, HCV Ab | HuPA29VH#1 | WO2007143701 SEQ ID NO: 18 | 25969 |
| HEPBD302 | Light chain, HCV Ab | HuPA29VH#2 | WO2007143701 SEQ ID NO: 19 | 25970 |
| HEPBD303 | Light chain, HCV Ab | PA29 | WO2007143701 SEQ ID NO: 29 | 25971 |
| HEPBD304 | Light chain, HCV Ab | Single Chain Fv | Gilmartin, A. A., et al., Protein Eng. Des. | 25972 |
| | | Fragment 1:7 | Sel. 25 (2), 59-66 (2012) NCBI Accession | |
| | | | # 3U6R_B (143aa) | |
| HEPBD305 | Light chain, HCV Ab | Igh526 | Kong L., et al., Structure of Hepatitis C | 25973 |
| | | | Virus Envelope Glycoprotein E1 Antigenic | |
| | | | Site 314-324 in Complex with Antibody | |
| | | | IGH526; J. Mol. Biol. 427 (16), 2617-2628 | |
| | | | (2015) NCBI Accession # 4N0Y_L | |
| | | | (218aa) | |
| HEPBD306 | Light chain, HCV Fab | Ar3c | Kong, L., et al., Hepatitis C virUSE2 | 25974 |
| | | | envelope glycoprotein core structure; | |
| | | | Science 342 (6162), 1090-1094 (2013) | |
| | | | NCBI Accession # 4MWF_B (214aa) | |
| HEPBD307 | Light chain, HCV Fab | Mrct10.v362 | Pantua, H., et al., Glycan shifting on | 25975 |
| | | | hepatitis C virus (HCV) e2 glycoprotein is | |
| | | | a mechanism for escape from broadly | |
| | | | neutralizing antibodies; J. Mol. Biol. 425 | |
| | | | (11), 1899-1914 (2013) NCBI Accession # | |
| | | | 4HS6_L (218aa) | |
| HEPBD308 | Light chain, Hcv1 | Hcv1, C2 Form | Kong, L., et al., Structural basis of | 25976 |
| | HCV Ab | | hepatitis C virus neutralization by broadly | |
| | | | neutralizing antibody HCV1; Proc. Natl. | |
| | | | Acad. Sci, U.S.A. 109 (24), 9499-9504 | |
| | | | (2012) NCBI Accession # 4DGY_L | |
| | | | (213aa) | |
| HEPBD309 | Light chain, Hcv1 | Hcv1, P2(1) | Kong, L., et al., Structural basis of | 25977 |
| | HCV Ab | Form | hepatitis C virus neutralization by broadly | |
| | | | neutralizing antibody HCV1; Proc. Natl. | |
| | | | Acad. Sci, U.S.A. 109 (24), 9499-9504 | |
| | | | (2012) NCBI Accession # 4DGV_L | |
| | | | (213aa) | |
| HEPBD310 | Light kappa chain | P18-9E | U.S. Pat. No. 8,592,559 SEQ ID NO: 14 | 25978 |
| | variable, HCV Ab | | | |
| HEPBD311 | PEGylated anti-E2 | | WO2006028634 SEQ ID NO: 1 | 25979 |
| | heavy chain, HCV Ab | | | |
| HEPBD312 | PEGylated anti-E2 | | WO2006028634 SEQ ID NO: 2 | 25980 |
| | heavy chain, HCV Ab | | | |
| HEPBD313 | PEGylated anti-E2 | | WO2006028634 SEQ ID NO: 3 | 25981 |
| | heavy chain, HCV Ab | | | |
| HEPBD314 | PEGylated anti-E2 | | WO2006028634 SEQ ID NO: 4 | 25982 |
| | heavy chain, HCV Ab | | | |
| HEPBD315 | PEGylated anti-E2 | | WO2006028634 SEQ ID NO: 8 | 25983 |
| | heavy chain, HCV Ab | | | |
| HEPBD316 | single chain, HBV Ab | | U.S. Pat. No. 6,562,599 SEQ ID NO: 4 | 25984 |
| HEPBD317 | single chain, HBV Ab | | U.S. Pat. No. 6,562,599 SEQ ID NO: 6 | 25985 |

In some embodiments, the payload region of the viral particle comprises one or more nucleic acid sequences, fragments or variants thereof or encodes one or more polypeptides, fragments or variants thereof described in U.S. Pat. Nos. 7,241,445, and 8,858,947, the contents of each of which are herein incorporated by reference in their entirety, against HCV.

In some embodiments, the payload region of the viral particle comprises one or more nucleic acid sequences, fragments or variants thereof or encodes one or more polypeptides, fragments or variants thereof described in US Publication No. US20150072885 and US20110046354, U.S. Pat. No. 5,204,095, European Publication No. EP0232921, EP0038642, and EP0186371, and International Publication No. WO1994011495, the contents of each of which are herein incorporated by reference in their entirety, against HBV.

In some embodiments, the payload region of the viral particle comprises one or more nucleic acid sequences, fragments or variants thereof or encodes one or more polypeptides, fragments or variants thereof described in U.S. Pat. No. 6,020,195, the contents of which are herein incorporated by reference in their entirety, against HGV (hepatitis (i virus).

In some embodiments, the payload region of the viral particle comprises one or more nucleic acid sequences encoding one or more of the payload antibody polypeptides listed in Table 35 against Herpes Virus (HERP1-HERP109; SEQ ID NO: 25986-26094).

TABLE 35
Antibodies against Herpesvirus

| Antibody | | Antibody | | SEQ |
|---|---|---|---|---|
| No. | Description | Name | Reference Information | ID NO |
| HERP1 | Chain A, HSV | E317 Fab | Lee, C. et al., “Structural basis for the | 25986 |
| | | | antibody neutralization of herpes simplex | |
| | | | virus” Acta Crystallogr. D Biol. Crystallogr. | |
| | | | 69 (PT 10), 1935-1945 (2013), NCBI | |
| | | | Accession # 3W9D_A | |
| HERP2 | Chain B, HSV | E317 Fab | Lee, C. et al., “Structural basis for the | 25987 |
| | | | antibody neutralization of herpes simplex | |
| | | | virus” Acta Crystallogr. D Biol. Clystallogr. | |
| | | | 69 (PT 10), 1935-1945 (2013), NCBI | |
| | | | Accession # 3W9D_B | |
| HERP3 | Chain C, HSV | E317 Fab | Lee, C. et al., “Structural basis for the | 25988 |
| | | | antibody neutralization of herpes simplex | |
| | | | virus” Acta Crystatlogr. D Biol. Crystallogr. | |
| | | | 69 (PT 10), 1935-1945 (2013), NCBI | |
| | | | Accession # 3W9D_C | |
| HERP4 | Chain D, HSV | E317 Fab | Lee, C. et al., “Structural basis for the | 25989 |
| | | | antibody neutralization of herpes simplex | |
| | | | virus” Acta Crystallogr. D Biol. Clystallogr. | |
| | | | 69 (PT 10), 1935-1945 (2013), NCBI | |
| | | | Accession # 3W9D_D | |
| HERP5 | Chimeric anti- | | Tanner, J. E., “Peptides Designed To Spatially | 25990 |
| | EBVs | | Depict the Epstein-Barr Virus Major Virion | |
| | gp350 antibody | | Glycoprotein gp350 Neutralization Epitope | |
| | | | Elicit Antibodies That Block Virus- | |
| | | | Neutralizing Antibody 72A1 Interaction with | |
| | | | the Native gp350 Molecule””, J. Virol. 89 (9), | |
| | | | 4932-4941 (2015), NCBI Accession | |
| | | | #AJR20276 | |
| HERP6 | Chimeric anti- | | Tanner, J. E., “Peptides Designed To Spatially | 25991 |
| | EBVs | | Depict the Epstein-Barr Virus Major Virion | |
| | gp350 antibody | | Glycoprotein gp350 Neutralization Epitope | |
| | | | Elicit Antibodies That Block Virus- | |
| | | | Neutralizing Antibody 72A1 Interaction with | |
| | | | the Native gp350 Molecule””, J. Virol. 89 (9), | |
| | | | 4932-4941 (2015), NCBI Accession | |
| | | | #AJR20275 | |
| HERP7 | CMV | AE11F/3-20L1 | Lantto, J. et al., Binding characteristics | 25992 |
| | | | determine the neutralizing potential of | |
| | | | antibody fragments specific for antigenic | |
| | | | domain 2 on glycoprotein B of human | |
| | | | cytomegalovirus, Virology 305 (1), 201-209 | |
| | | | (2003), NCBI Accession # AAN87569.1 (256 | |
| | | | aa) | |
| HERP8 | Fv, EBV | G5 | Fang, C. Y., “Modulation of Epstein-Barr virus | 25993 |
| | | | latent membrane protein 1 activity by | |
| | | | intrabodies”, Intervirology 50 (4), 254-263 | |
| | | | (2007), NCBI Accession #ABA55015 | |
| HERP9 | Fv, EBV | A4 | Fang, C. Y., “Modulation of Epstein-Barr virus | 25994 |
| | | | latent membrane protein 1 activity by | |
| | | | intrabodies”, Intervirology 50 (4), 254-263 | |
| | | | (2007), NCBI Accession #ABA55014 | |
| HERP10 | Fv, EBV | B8 | Fang, C. Y., “Modulation of Epstein-Barr virus | 25995 |
| | | | latent membrane protein 1 activity by | |
| | | | intrabodies”, Intervirology 50 (4), 254-263 | |
| | | | (2007), NCBI Accession #ABA55013 | |
| HERP11 | Fv, EBV | F5 | Fang, C. Y., “Modulation of Epstein-Barr virus | 25996 |
| | | | latent membrane protein 1 activity by | |
| | | | intrabodies”, Intervirology 50 (4), 254-263 | |
| | | | (2007), NCBI Accession #ABA55012 | |
| HERP12 | Fv, EBV | E2 | Fang, C. Y., “Modulation of Epstein-Barr virus | 25997 |
| | | | latent membrane protein 1 activity by | |
| | | | intrabodies”, Intervirology 50 (4), 254-263 | |
| | | | (2007), NCBI Accession #ABA55011 | |
| HERP13 | Fv, EBV | H3 | Fang, C. Y., “Modulation of Epstein-Barr virus | 25998 |
| | | | latent membrane protein 1 activity by | |
| | | | intrabodies”, Intervirology 50 (4), 254-263 | |
| | | | (2007), NCBI Accession #ABA55010 | |
| HERP14 | Heavy chain, | DDF-VZV1 | US20100074906 SEQ ID NO: 20 | 25999 |
| | FLAGhis tagged | | | |
| | sequence, VZV | | | |
| HERP15 | Heavy chain | ACHDV1 | Burioni, R. et al. “Recombinant human Fab to | 26000 |
| | variable domain, | | glycoprotein D neutralizes infectivity and | |
| | clone 11, HSV | | prevents cell-to-cell transmission of herpes | |
| | | | simplex viruses 1 and 2 in vitro”, Proc. Natl. | |
| | | | Acad. Sci. U.S.A. 91 (1), 355-359 (1994), | |
| | | | NCBI Accession # AAB29447 | |
| HERP16 | Heavy chain | ACHDV1 | Burioni, R. et al. “Recombinant human Fab to | 26001 |
| | variable domain, | | glycoprotein D neutralizes infectivity and | |
| | clone 13, HSV | | prevents cell-to-cell transmission of herpes | |
| | | | simplex viruses 1 and 2 in vitro”, Proc. Natl. | |
| | | | Acad. Sci. U.S.A. 91 (1), 355-359 (1994), | |
| | | | NCBI Accession # AAB29449 | |
| HERP17 | Heavy chain | ACHDV2 | Burioni, R, et al, “Recombinant human Fab to | 26002 |
| | variable domain, | | glycoprotein D neutralizes infectivity and | |
| | clone 15, HSV | | prevents cell-to-cell transmission of herpes | |
| | | | simplex viruses 1 and 2 in vitro”, Proc. Natl. | |
| | | | Acad. Sci. U.S.A. 91 (1), 355-359 (1994), | |
| | | | NCBI Accession # AAB29456 | |
| HERP18 | Heavy chain | ACHDV1 | Burioni, R. et al. “Recombinant human Fab to | 26003 |
| | variable domain, | | glycoprotein D neutralizes infectivity and | |
| | clone 15, HSV | | prevents cell-to-cell transmission of herpes | |
| | | | simplex viruses 1 and 2 in vitro”, Proc. Natl. | |
| | | | Acad. Sci. U.S.A. 91 (1), 355-359 (1994), | |
| | | | NCBI Accession # AAB29450 | |
| HERP19 | Heavy chain | ACHDV1 | Burioni, R. et al. “Recombinant human Fab to | 26004 |
| | variable domain, | | glycoprotein D neutralizes infectivity and | |
| | clone 18, HSV | | prevents cell-to-cell transmission of herpes | |
| | | | simplex viruses 1 and 2 in vitro”, Proc. Natl. | |
| | | | Acad. Sci. U.S.A. 91 (1), 355-359 (1994), | |
| | | | NCBI Accession # AAB29448 | |
| HERP20 | Heavy chain | ACHDV1 | Burioni, R. et al. “Recombinant human Fab to | 26005 |
| | variable domain, | | glycoprotein D neutralizes infectivity and | |
| | clone 2, HSV | | prevents cell-to-cell transmission of herpes | |
| | | | simplex viruses 1 and 2 in vitro”, Proc. Natl. | |
| | | | Acad. Sci. U.S.A. 91 (1), 355-359 (1994), | |
| | | | NCBI Accession # AAB29455 | |
| HERP21 | Heavy chain | IF7 | U.S. Pat. No. 8,202,518 SEQ ID NO: 5 | 26006 |
| | variable region, | | | |
| | CMV | | | |
| HERP22 | Heavy chain | Humanized 57.4 | WO2014200898 SEQ ID NO: 633 | 26007 |
| | variable region, | | | |
| | CMV | | | |
| HERP23 | Heavy chain | Humanized 57.4 | WO2014200898 SEQ ID NO: 634 | 26008 |
| | variable region, | | | |
| | CMV | | | |
| HERP24 | Heavy chain | Humanized 58.5 | WO2014200898 SEQ ID NO: 637 | 26009 |
| | variable region, | | | |
| | CMV | | | |
| HERP25 | Heavy chain | Humanized 58.5 | WO2014200898 SEQ ID NO: 638 | 26010 |
| | variable region, | | | |
| | CMV | | | |
| HERP26 | Heavy chain | Humanized | WO2014200898 SEQ ID NO: 641 | 26011 |
| | variable region, | 272.7 | | |
| | CMV | | | |
| HERP27 | Heavy chain | Humanized | WO2014200898 SEQ ID NO: 644 | 26012 |
| | variable region, | 276.10 | | |
| | CMV | | | |
| HERP28 | Heavy chain | Humanized | WO2014200898 SEQ ID NO: 645 | 26013 |
| | variable region, | 276.10 | | |
| | CMV | | | |
| HERP29 | Heavy chain | Sm5-1 | Li, B., Construction and characterization of a | 26014 |
| | variable region, | | high-affinity humanized SMS-1 monoclonal | |
| | CMV | | antibody, Biochem. Biophys. Res. Commun. | |
| | | | 357 (4), 951-956 (2007), NCBI Accession # | |
| | | | ABI22831.1 | |
| HERP30 | Heavy chain | | Schoppel, K. et al., Antibodies specific for the | 26015 |
| | variable region, | | antigenic domain 1 of glycoprotein B | |
| | CMV | | (gpUL55) of human cytomegalovirus bind to | |
| | | | different substructures, Virology 216 (1), 133- | |
| | | | 145 (1996), NCBI Accession # AAB26953.1 | |
| | | | (163 aa) | |
| HERP31 | Heavy chain | | Schoppel, K. et al., Antibodies specific for the | 26016 |
| | variable region, | | antigenic domain 1 of glycoprotein B | |
| | CMV | | (gpUL55) of human cytomegalovirus bind to | |
| | | | different substructures, Virology 216 (1), 133- | |
| | | | 145 (1996), NCBI Accession # AAB26952.1 | |
| | | | (161 aa) | |
| HERP32 | Heavy chain | | Schoppel, K. et al., Antibodies specific for the | 26017 |
| | variable region, | | antigenic domain 1 of glycoprotein B | |
| | CMV | | (gpUL55) of human cytomegalovirus bind to | |
| | | | different substructures, Virology 216 (1), 133- | |
| | | | 145 (1996), NCBI Accession # AAB26951.1 | |
| | | | (158 aa) | |
| HERP33 | Heavy chain | | Potzsch, S., B Cell Repertoire Analysis | 26018 |
| | variable region, | | Identifies New Antigenic Domains on | |
| | CMV | | Glycoprotein B of Human Cytomegalovirus | |
| | | | which Are Target of Neutralizing Antibodies, | |
| | | | NCBI Accession # AEF33814.1 | |
| HERP34 | Heavy chain | 1F11 | U.S. Pat. No. 9,149,524 SEQ ID NO: 7 | 26019 |
| | variable region, | | | |
| | CMV, a complex | | | |
| | of human | | | |
| | cytomegalovirus | | | |
| | (hCMV) proteins | | | |
| | UL130 and | | | |
| | UL131A | | | |
| HERP35 | Heavy chain | 2F4 | U.S. Pat. No. 9,149,524 SEQ ID NO: 17 | 26020 |
| | variable region, | | | |
| | CMV, a complex | | | |
| | of human | | | |
| | cytomegalovirus | | | |
| | (hCMV) proteins | | | |
| | UL130 and | | | |
| | UL131A | | | |
| HERP36 | Heavy chain | 5A2 | U.S. Pat. No. 9,149,524 SEQ ID NO: 39 | 26021 |
| | variable region, | | | |
| | CMV, a complex | | | |
| | of human | | | |
| | cytomegalovirus | | | |
| | (hCMV) proteins | | | |
| | UL130 and | | | |
| | UL131A | | | |
| HERP37 | Heavy chain | EV2038 | U.S. Pat. No. 8,492,529 SEQ ID NO: 10 | 26022 |
| | variable region, | | | |
| | CMV, AD1 | | | |
| | region of HMV | | | |
| | glycoprotein gB | | | |
| HERP38 | Heavy chain | | US20150064174 SEQ ID 1 | 26023 |
| | variable region, | | | |
| | EBV | | | |
| HERP39 | Heavy chain | | US20150064174 SEQ ID 2 | 26024 |
| | variable region, | | | |
| | EBV | | | |
| HERP40 | Heavy chain | HCMV16 | WO1994009136, FIG. 1 | 26025 |
| | variable region, | | | |
| | gH glycoprotein | | | |
| | of HCMV | | | |
| HERP41 | Heavy chain | | Nejatollahi, F. and Bagheri, V., “Isolation of | 26026 |
| | variable region, | | neutralizing human specific single-chain | |
| | HSV | | antibodies against Herpes Simplex Virus type | |
| | | | 1 glycoproiein D”, unpublished, NCBI | |
| | | | Accession # AGO59015 | |
| HERP42 | Heavy chain | E317 | U.S. Pat. No. 8,431,118 SEQ ID NO: 1; U.S. Pat. No. 8,252,906 | 26027 |
| | variable region, | | | |
| | HSV 1&2 | | | |
| HERP43 | Heavy chain | E425 | U.S. Pat. No. 8,431,118 SEQ ID NO: 3; U.S. Pat. No. 8,252,906 | 26028 |
| | variable region, | | | |
| | HSV 1&2 | | | |
| HERP44 | Heavy chain | Y571 | U.S. Pat. No. 8,431,118 SEQ ID NO: 41; U.S. Pat. No. 8,252,906 | 26029 |
| | variable region, | | | |
| | HSV 1&2 | | | |
| HERP45 | Heavy chain | | U.S. Pat. No. 5,506,132 SEQ ID NO: 4 | 26030 |
| | variable region, | | | |
| | VZV | | | |
| HERP46 | Heavy chain | DDF-VZV2 | US20100074906 SEQ ID NO: 26 | 26031 |
| | variable region, | | | |
| | VZV | | | |
| HERP47 | Heavy chain | EV2038 | U.S. Pat. No. 8,492,529 SEQ ID NO: 6 | 26032 |
| | without a signal | | | |
| | sequence, CMV, | | | |
| | AD1 region of | | | |
| | HCMV | | | |
| | glycoprote in gB | | | |
| HERP48 | Heavy chain, | 8f9 | McLean, G. R. et al., Recognition of human | 26033 |
| | CMV | | cytomegalovirus by human primary | |
| | | | immunoglobulins identifies an innate | |
| | | | foundation to an adaptive immune response, J. | |
| | | | Immunol. 174 (8), 4768-4778 (2005), NCBI | |
| | | | Accession # CAE54374.1 | |
| HERP49 | Heavy chain, | Mab 109 | Simpson, J. A. et al., Neutralizing monoclonal | 26034 |
| | CMV | | antibodies that distinguish three antigenic sites | |
| | | | on human cytomegalovirus glycoprotein H | |
| | | | have conformationally distinct binding sites, J. | |
| | | | Virol. 67 (1), 489-496 (1993), NCBI | |
| | | | Accession # AAB24505.1 (119 aa) | |
| HERP50 | Heavy chain, | Mab 115 | Simpson, J. A. et al., Neutralizing monoclonal | 26035 |
| | CMV | | antibodies that distinguish three antigenic sites | |
| | | | on human cytomegalovirus glycoprotein H | |
| | | | have conformationaly distinct binding sites, J. | |
| | | | Virol. 67 (1), 489-496 (1993), NCBI | |
| | | | Accession # AAB24504.1 (117 aa) | |
| HERP51 | Heavy chain, | Mab 33 | Simpson, J. A. et al., Neutralizing monoclonal | 26036 |
| | CMV | | antibodies that distinguish three antigenic sites | |
| | | | on human cytomegalovirus glycoprotein H | |
| | | | have conformationally distinct binding sites, J. | |
| | | | Virol. 67 (1), 489-496 (1993), NCBI | |
| | | | Accession # AAB24503.1 (120 aa) | |
| HERP52 | Heavy chain, | Mab 5 | Simpson, J. A. et at., Neutralizing monoclonal | 26037 |
| | CMV | | antibodies that distinguish three antigenic sites | |
| | | | on human cytomegalovirus glycoprotein H | |
| | | | have conformationaly distinct binding sites, J. | |
| | | | Virol. 67 (1), 489-496 (1993), NCBI | |
| | | | Accession # AAB24502.1 (120 aa) | |
| HERP53 | Heavy chain, | 6G4 | WO2010007463 SEQ ID NO: 7 | 26038 |
| | CMV, a | | | |
| | combination of | | | |
| | the hCMV | | | |
| | proteins UL128, | | | |
| | UL130 and | | | |
| | UL131A | | | |
| HERP54 | Heavy chain, | | US20140093526 SEQ ID 12 | 26039 |
| | HHV-6 | | | |
| HERP55 | Heavy chain, | FabHSV 8. | U.S. Pat. No. 6,156,313 SEQ ID NO: 2 | 26040 |
| | HSV 1&2 | | | |
| HERP56 | Heavy chain, | 64-683 | U.S. Pat. No. 5,646,041 SEQ ID NO: 2; EP876478 | 26041 |
| | HSV 1&2 | | | |
| HERP57 | Heavy chain, | H005157 | US20140302062 SEQ ID NO: 3 | 26042 |
| | HSV 1&2 | | | |
| HERP58 | Heavy chain, | H005158 | US20140302062 SEQ ID NO: 4 | 26043 |
| | HSV 1&2 | | | |
| HERP59 | Heavy chain, | H005159 | US20140302062 SEQ ID NO: 5 | 26044 |
| | HSV 1&2 | | | |
| HERP60 | Heavy chain, | H005160 | US20140302062 SEQ ID NO: 6 | 26045 |
| | HSV 1&2 | | | |
| HERP61 | Heavy chain, | H005188 | US20140302062 SEQ ID NO: 7 | 26046 |
| | HSV 1&2 | | | |
| HERP62 | Heavy chain, | H005190 | US20140302062 SEQ ID NO: 8 | 26047 |
| | HSV 1&2 | | | |
| HERP63 | Heavy chain, | H005192 | US20140302062 SEQ ID NO: 9 | 26048 |
| | HSV 1&2 | | | |
| HERP64 | Light chain | HCMV16 | WO1994009136, FIG. 2 | 26049 |
| | variable region, | | | |
| | gH glycoprotein | | | |
| | of HCMV | | | |
| HERP65 | Light chain | DDF-VZV1 | US20100074906 SEQ ID NO: 22 | 26050 |
| | recombinant, | | | |
| | VZV | | | |
| HERP66 | Light chain | 1F7 | U.S. Pat. No. 8,202,518 SEQ ID NO: 10 | 26051 |
| | variable region, | | | |
| | CMV | | | |
| HERP67 | Light chain | Humanized 57.4 | WO2014200898 SEQ ID NO: 631 | 26052 |
| | variable region, | | | |
| | CMV | | | |
| HERP68 | Light chain | Humanized 57.4 | WO2014200898 SEQ ID NO: 632 | 26053 |
| | variable region, | | | |
| | CMV | | | |
| HERP69 | Light chain | Humanized 58.5 | WO2014200898 SEQ ID NO: 635 | 26054 |
| | variable region, | | | |
| | CMV | | | |
| HERP70 | Light chain | Humanized 58.5 | WO2014200898 SEQ ID NO: 636 | 26055 |
| | variable region, | | | |
| | CMV | | | |
| HERP71 | Light chain | Humanized | WO2014200898 SEQ ID NO: 639 | 26056 |
| | variable region, | 272.7 | | |
| | CMV | | | |
| HERP72 | Light chain | Humanized | WO2014200898 SEQ ID NO: 640 | 26057 |
| | variable region, | 272.7 | | |
| | CMV | | | |
| HERP73 | Light chain | Humanized | WO2014200898 SEQ ID NO: 642 | 26058 |
| | variable region, | 276.10 | | |
| | CMV | | | |
| HERP74 | Light chain | Humanized | WO2014200898 SEQ ID NO: 643 | 26059 |
| | variable region, | 276.10 | | |
| | CMV | | | |
| HERP75 | Light chain | Sm5-1 | Li, B., Construction and characterization of a | 26060 |
| | variable region, | | high-affinity humanized SM5-1 monoclonal | |
| | CMV | | antibody, Biochem. Biophys. Res. Commun. | |
| | | | 357 (4), 951-956 (2007), NCBI Accession # | |
| | | | ABI22832.1 | |
| HERP76 | Light chain | 8f9 | Schoppel, K. et al., Antibodies specific for the | 26061 |
| | variable region, | | antigenic domain 1 of glycoprotein B | |
| | CMV | | (gpUL55) of human cytomegalovirus bind to | |
| | | | different substructures, Virology 216 (1), 133- | |
| | | | 145 (1996), NCBI Accession # AAB26956.1 | |
| | | | (146 aa) | |
| HERP77 | Light chain | | Schoppel, K. et al., Antibodies specific for the | 26062 |
| | variable region, | | antigenic domain 1 of glycoprotein B | |
| | CMV | | (gpUL55) of human cytomegalovirus bind to | |
| | | | different substructures, Virology 216 (1), 133- | |
| | | | 145 (1996), NCBI Accession # AAB26955.1 | |
| | | | (141 aa) | |
| HERP78 | Light chain | | Schoppel, K. et al., Antibodies specific for the | 26063 |
| | variable region, | | antigenic domain 1 of glycoprotein B | |
| | CMV | | (gpUL55) of human cytomegalovirus bind to | |
| | | | different substructures, Virology 216 (1), 133- | |
| | | | 45 (1996), NCBI Accession # AAB26954.1 | |
| | | | (152 aa) | |
| HERP79 | Light chain | | Potzsch, S., B Cell Repertoire Analysis | 26064 |
| | variable region, | | Identifies New Antigenic Domains on | |
| | CMV | | Glycoprotein B of Human Cytomegalovirus | |
| | | | which Are Target of Neutralizing Antibodies, | |
| | | | NCBI Accession # AEF33824.1 | |
| HERP80 | Light chain | 1F11 | U.S. Pat. No. 9,149,524 SEQ ID NO: 8 | 26065 |
| | variable region, | | | |
| | CMV, a | | | |
| | combination of | | | |
| | the hCMV | | | |
| | proteins UL128, | | | |
| | UL130 and | | | |
| | UL131A | | | |
| HERP81 | Light chain | 2F4 | U.S. Pat. No. 9,149,524 SEQ ID NO: 18 | 26066 |
| | variable region, | | | |
| | CMV, a | | | |
| | combination of | | | |
| | the hCMV | | | |
| | proteins UL128, | | | |
| | UL130 and | | | |
| | UL131A | | | |
| HERP82 | Light chain | 5A2 | U.S. Pat. No. 9,149,524 SEQ ID NO: 40 | 26067 |
| | variable region, | | | |
| | CMV, a | | | |
| | combination of | | | |
| | the hCMV | | | |
| | proteins UL128, | | | |
| | UL130 and | | | |
| | UL131A | | | |
| HERP83 | Light chain | EV2038 | U.S. Pat. No. 8,492,529 SEQ ID NO. 12 | 26068 |
| | variable region, | | | |
| | CMV, AD1 | | | |
| | region of HCMV | | | |
| | glycoprotein gB | | | |
| HERP84 | Light chain | | US20150064174 SEQ ID 3 | 26069 |
| | variable region, | | | |
| | EBV | | | |
| HERP85 | Light chain | | US20150064174 SEQ ID 4 | 26070 |
| | variable region, | | | |
| | EBV | | | |
| HERP86 | Light chain | | Nejatollahi, F. and Bagheri, V., “Isolation of | 26071 |
| | variable region, | | neutralizing human specific single-chain | |
| | HSV | | antibodies against Herpes Simplex Virus type | |
| | | | 1 glycoprotein D”, unpublished”, NCBI | |
| | | | Accession # AGO59016 | |
| HERP87 | Light chain | E317 | U.S. Pat. No. 8,431,118 SEQ ID NO: 2; U.S. Pat. No. 8,252,906 | 26072 |
| | variable region, | | | |
| | HSV 1&2 | | | |
| HERP88 | Light chain | E425 | U.S. Pat. No. 8,431,118 SEQ ID NO: 4; U.S. Pat. No. 8,252,906 | 26073 |
| | variable region, | | | |
| | HSV 1&2 | | | |
| HERP89 | Light chain | Y571 | U.S. Pat. No. 8,431,118 SEQ ID NO: 42; U.S. Pat. No. 8,252,906 | 26074 |
| | variable region, | | | |
| | HSV 1&2 | | | |
| HERP90 | Light chain | | U.S. Pat. No. 5,506,132 SEQ ID NO: 2 | 26075 |
| | variable region, | | | |
| | VZV | | | |
| HERP91 | Light chain | DDF-VZV2 | US20100074906 SEQ ID NO: 24 | 26076 |
| | variable region, | | | |
| | VZV | | | |
| HERP92 | Light chain | EV2038 | U.S. Pat. No. 8,492,529 SEQ ID NO: 8 | 26077 |
| | without a signal | | | |
| | sequence, CMV, | | | |
| | AD1 region of | | | |
| | HCMV | | | |
| | glycoprotein gB | | | |
| HERP93 | Light chain, CMV | 8f9 | McLean, G. R. et al., Recognition of human | 26078 |
| | | | cytomegalovirus by human primary | |
| | | | immunoglobulins identifies an innate | |
| | | | foundation to an adaptive immune response, J. | |
| | | | Immunol. 174 (8), 4768-4778 (2005), NCBI | |
| | | | Accession # CAE54366.1 | |
| HERP94 | Light chain, CMV | Mab 109 | Simpson, J. A. et al., Neutralizing monoclonal | 26079 |
| | | | antibodies that distinguish three antigenic sites | |
| | | | on human cytomegalovirus glycoprotein H | |
| | | | have conformationally distinct binding sites, J. | |
| | | | Virol. 67 (1), 489-496 (1993), NCBI | |
| | | | Accession # AAB24501.1 (111 aa) | |
| HERP95 | Light chain, CMV | Mab 115 | Simpson, J. A. et al., Neutralizing monoclonal | 26080 |
| | | | antibodies that distinguish three antigenic sites | |
| | | | on human cytomegalovirus glycoprotein H | |
| | | | have conformationaly distinct binding sites, J. | |
| | | | Virol. 67 (1), 489-496 (1993), NCBI | |
| | | | Accession # AAB24500.1 (107 aa) | |
| HERP96 | Light chain, CMV | Mab 33 | Simpson, J. A. et al., Neutralizing monoclonal | 26081 |
| | | | antibodies that distinguish three antigenic sites | |
| | | | on human cytomegalovirus glycoprotein H | |
| | | | have conformationally distinct binding sites, J. | |
| | | | Virol. 67 (1), 489-496 (1993), NCBI | |
| | | | Accession # AAB24499.1 (107 aa) | |
| HERP97 | Light chain, CMV | Mab 5 | Simpson, J. A. et al., Neutralizing monoclonal | 26082 |
| | | | antibodies that distinguish three antigenic sites | |
| | | | on human cytomegalovirus glycoprotein H | |
| | | | have conformationally distinct binding sites, J. | |
| | | | Virol. 67 (1), 489-496 (1993), NCBI | |
| | | | Accession # AAB24498.1 (107 aa) | |
| HERP98 | Light chain, | 6G4 | WO2010007463 SEQ ID NO: 8 | 26083 |
| | CMV, a | | | |
| | combination of | | | |
| | the hCMV | | | |
| | proteins UL128, | | | |
| | UL130 and | | | |
| | UL131A | | | |
| HERP99 | Light chain, | | US20140093526 SEQ ID 10 | 26084 |
| | HHV-6 | | | |
| HERP100 | Light chain, HSV | 64-683 | U.S. Pat. No. 5,646,041 SEQ ID NO: 4; EP876478 | 26085 |
| | 1&2 | | | |
| HERP101 | Light chain, HSV | K003927 | US20140302062 SEQ ID NO: 10 | 26086 |
| | 1&2 | | | |
| HERP102 | Light chain, HSV | K003928 | US20140302062 SEQ ID NO: 11 | 26087 |
| | 1&2 | | | |
| HERP103 | Light chain, HSV | K003929 | US20140302062 SEQ ID NO: 12 | 26088 |
| | 1&2 | | | |
| HERP104 | Light chain, HSV | K003930 | US20140302062 SEQ ID NO: 13 | 26089 |
| | 1&2 | | | |
| HERP105 | Light chain, HSV | K003946 | US20140302062 SEQ ID NO: 14 | 26090 |
| | 1&2 | | | |
| HERP106 | Light chain, HSV | K003948 | US20140302062 SEQ ID NO: 15 | 26091 |
| | 1&2 | | | |
| HERP107 | Light chain, HSV | K003949 | US20140302062 SEQ ID NO: 16 | 26092 |
| | 1&2 | | | |
| HERP108 | Light chain, HSV | L001844 | US20140302062 SEQ ID NO: 17 | 26093 |
| | 1&2 | | | |
| HERP109 | Single chain Fv | | Lantto, J. et al., Non-germ-line encoded | 26094 |
| | antibody, | | residues are critical for effective antibody | |
| | glycoprotein B | | recognition of a poorly immunogenic | |
| | recombinant, | | neutralization epitope on glycoprotein B of | |
| | CMV | | human cytomegalovirus, Eur. J. Immunol. 32 | |
| | | | (6), 1659-1669 (2002), NCBI Accession # | |
| | | | AAM92769.1 (255 aa) | |

In some embodiments, the payload region of the viral particle comprises one or more nucleic acid sequences, fragment or variants thereof or encodes one or more polypeptides, fragments or variants thereof described in International Publication No. WO2010109874, and WO1997026329, the contents of each of which are herein incorporated by reference in their entirety, against HSV.

In some embodiments, the payload region of the viral particle comprises one or more nucleic acid sequences, fragment or variants thereof or encodes one or more polypeptides, fragments or variants thereof described in International Publication No. WO1995031546, the contents of which are herein incorporated by reference in their entirety, against VZV.

In some embodiments, the payload region of the viral particle comprises one or more nucleic acid sequences encoding one or more of the payload antibody polypeptides listed in Table 36 against Coronavirus (CORV1-CORV65; SEQ ID NO: 26095-26159).

TABLE 36
Antibodies against Coronaviruses

| Antibody | | Antibody | Reference | SEQ |
|---|---|---|---|---|
| No. | Description | Name | Information | ID NO |
| CORV1 | Heavy chain partial sequence, | | Liu, J., unpublished, NCBI | 26095 |
| | Human anti-SARS antibody, Ig | | Accession # BAE94186.1(228aa) | |
| CORV2 | Heavy chain partial sequence | H12 | AAX19356.1(127aa) | 26096 |
| | Human SARS neutralization | | | |
| | antibody, Ig | | | |
| CORV3 | Heavy chain variable partial | | Leung et at., PLoS Med. 3 (7), | 26097 |
| | sequence, Human neutralizing | | E237 (2006), NCBI Accession # | |
| | SARS antibody | | ABA54614.1(113aa) | |
| CORV4 | Heavy chain variable region, | M396 | Prabakaran et al., J. Biol. Chem. | 26098 |
| | Human anti-SARS antibody | | 281 (23), 15829-15836 (2006), | |
| | | | NCBI Accession # 2G75_D | |
| | | | (213aa) | |
| CORV5 | Heavy chain variable region, | | Prabakaran et al., J. Biol. Chem. | 26099 |
| | Human neutralizing SARS | | 281 (23), 15829-15836 (2006), | |
| | antibody | | NCBI Accession # 2DD8_L | |
| | | | (213aa) | |
| CORV6 | Heavy chain variable region, | | CN103864924 SEQ ID NO: 1 | 26100 |
| | Humanized neutralizing murine | | | |
| | monoclonal MERS | | | |
| CORV7 | Heavy chain variable region, | | CN104447986 SEQ ID NO: 1 | 26101 |
| | MERs | | | |
| CORV8 | Heavy chain variable region, | | U.S. Pat. No. 7,750,123 | 26102 |
| | Neutralizing antibody (binds to | | SEQ ID NO: 12; | |
| | the spike protein (5) of SARS- | | WO2005060520; CN1914226; | |
| | cov) | | US20050249739 | |
| CORV9 | Heavy chain variable region, | s110.4 | US20110159001 SEQ ID NO: 62; | 26103 |
| | SARS antibody | | WO2009128963; EP2242768; | |
| | | | CN102015767 | |
| CORV10 | Heavy chain variable region, | s124.5 | US20110159001 SEQ ID NO: 66; | 26104 |
| | SARS antibody | | WO2009128963; EP2242768; | |
| | | | CN102015767 | |
| CORV11 | Heavy chain variable region, | s215.17 | US20110159001 SEQ ID NO: 70; | 26105 |
| | SARS antibody | | WO2009128963; EP2242768; | |
| | | | CN102015767 | |
| CORV12 | Heavy chain variable region, | s218.9 | US20110159001 SEQ ID NO: 74; | 26106 |
| | SARS antibody | | WO2009128963; EP2242768; | |
| | | | CN102015767 | |
| CORV13 | Heavy chain variable region, | s223.4 | US20110159001 SEQ ID NO: 78; | 26107 |
| | SARS antibody | | WO2009128963; EP2242768; | |
| | | | CN102015767 | |
| CORV14 | Heavy chain variable region, | s225.12 | US20110159001 SEQ ID NO: 82; | 26108 |
| | SARS antibody | | WO2009128963; EP2242768; | |
| | | | CN102015767 | |
| CORV15 | Heavy chain variable region, | s231.19 | US20110159001 SEQ ID NO: 86; | 26109 |
| | SARS antibody | | WO2009128963; EP2242768; | |
| | | | CN102015767 | |
| CORV16 | Heavy chain variable region, | s230.14 + 15 | US20110159001 SEQ ID NO: 90; | 26110 |
| | SARS antibody | | WO2009128963; EP2242768; | |
| | | | CN102015767 | |
| CORV17 | Heavy chain variable region, | s227.14 | US20110159001 SEQ ID NO: 94; | 26111 |
| | SARS antibody | | WO2009128963; EP2242768; | |
| | | | CN102015767 | |
| CORV18 | Heavy chain variable region, | s109.8 | US20110159001 SEQ ID NO: 98; | 26112 |
| | SARS antibody | | WO2009128963; EP2242768; | |
| | | | CN102015767 | |
| CORV19 | Heavy chain variable region, | Fab58 | CN1513874 | 26113 |
| | SARS antibody | | | |
| CORV20 | Heavy chain variable region, | Fab59 | CN1513874 | 26114 |
| | SARS antibody | | | |
| CORV21 | Heavy chain variable region, | 3C7 | U.S. Pat. No. 7,728,110 SEQ | 26115 |
| | SARS human monoclonal | | ID NO: 60; WO2008060331; | |
| | antibody | | EP2035454A2, US20080248043 | |
| CORV22 | Heavy chain variable region, | F26G18 | U.S. Pat. No. 7,622,112 SEQ | 26116 |
| | SARS human monoclonal | | ID NO: 5; WO2005054469; | |
| | antibody | | US20080248043; US20080081047 | |
| CORV23 | Heavy chain variable region A, | | WO2006095180 SEQ ID NO: 24 | 26117 |
| | humanized antibody binding to | | | |
| | S2 domain of SARS | | | |
| CORV24 | Heavy chain Humanized | | CN103864924 SEQ ID NO: 3 | 26118 |
| | neutralizing murine monoclonal | | | |
| | MERS | | | |
| CORV25 | Heavy chain, MERS | m336 | WO2015057942 SEQ ID NO: 1 | 26119 |
| CORV26 | Heavy chain, MERS | m337 | WO2015057942 SEQ ID NO: 9 | 26120 |
| CORV27 | Heavy chain, MERS | m338 | WO2015057942 SEQ ID NO: 16 | 26121 |
| CORV28 | Heavy chain, MERS | 2e 6 | CN104447986 SEQ ID NO: 3 | 26122 |
| CORV29 | Heavy chain, MERS | M336 | Ying et al., Nat Commun 6, 8223 | 26123 |
| | | | (2015), NCBI Accession # | |
| | | | 4XAK_H(252aa) | |
| CORV30 | Human anti-SARS antibody | | Leung et al., PLoS Med. 3 (7), | 26124 |
| | | | E237 (2006), NCBI Accession # | |
| | | | ABA54613.1(117aa) | |
| CORV31 | Human monoclonal MERS | Mers-27 | CN104628848 SEQ ID NO: 1 | 26125 |
| CORV32 | Human monoclonal MERS | Mers-27 | CN104628848 SEQ ID NO. 3 | 26126 |
| CORV33 | Human monoclonal MERS | Mers-4 | CN104628849 SEQ ID NO: 1 | 26127 |
| CORV34 | Human monoclonal MERS | Mers-4 | CN104628849 SEQ ID NO: 3 | 26128 |
| CORV35 | Kappa light chain partial | H12 | AX19355.1(108aa) | 26129 |
| | sequence, human SARS | | | |
| | neutralization antibody, Ig | | | |
| CORV36 | Light chain partial sequence, | | Liu, J., unpublished, NCBI | 26130 |
| | Human anti-SARS antibody, Ig | | Accession # BAE94187.1(219aa) | |
| CORV37 | Light chain variable domain, | | CN104447986 SEQ ID NO: 2 | 26131 |
| | MERS | | | |
| CORV38 | Light chain variable partial | 80R | Hwang et al., J. Biol. Chem. 281 | 26132 |
| | sequence, Human neutralizing | | (45), 34610-34616 (2006), NCBI | |
| | SARS antibody | | Accession # 2GHW_D (247aa) | |
| CORV39 | Light chain variable region, A | | WO2006095180 SEQ ID NO: 25 | 26133 |
| | humanized antibody binding to | | | |
| | S2 domain of SARS | | | |
| CORV40 | Light chain variable region, | M396 | Prabakaran et al., J. Biol. Chem. | 26134 |
| | human anti-SARS antibody | | 281 (23), 15829-15836 (2006), | |
| | | | NCBI Accession # 2G75_C | |
| | | | (245aa) | |
| CORV41 | Light chain variable region, | | Prabakaran et al., J. Biol. Chem. | 26135 |
| | Human neutralizing SARS | | 281 (23), 15829-15836 (2006), | |
| | antibody | | NCBI Accession # | |
| | | | 2DD8_H(245aa) | |
| CORV42 | Light chain variable region, | | CN103864924 SEQ ID NO: 2 | 26136 |
| | Humanized neutralizing murine | | | |
| | monoclonal MERS | | | |
| CORV43 | Light chain variable region, | | U.S. Pat. No. 7,750,123 SEQ | 26137 |
| | neutralizing antibody (binds to | | ID NO: 20; WO2005060520; | |
| | the spike protein (S) of SARS- | | CN1914226; US20050249739 | |
| | cov) | | | |
| CORV44 | Light chain variable region, | s110.4 | US20110159001 SEQ ID NO: 64; | 26138 |
| | SARS antibody | | WO2009128963; EP2242768; | |
| | | | CN102015767 | |
| CORV45 | Light chain variable region, | s124.5 | US20110159001 SEQ ID NO: 68; | 26139 |
| | SARS antibody | | WO2009128963; EP2242768; | |
| | | | CN102015767 | |
| CORV46 | Light chain variable region, | s215.17 | US20110159001 SEQ ID NO: 72; | 26140 |
| | SARS antibody | | WO2009128963; EP2242768; | |
| | | | CN102015767 | |
| CORV47 | Light chain variable region, | s218.9 | US20110159001 SEQ ID NO: 76; | 26141 |
| | SARS antibody | | WO2009128963; EP2242768; | |
| | | | CN102015767 | |
| CORV48 | Light chain variable region, | s223.4 | US20110159001 SEQ ID NO: 80; | 26142 |
| | SARS antibody | | WO2009128963; EP2242768; | |
| | | | CN102015767 | |
| CORV49 | Light chain variable region, | s225.12 | US20110159001 SEQ ID NO: 84; | 26143 |
| | SARS antibody | | WO2009128963; EP2242768; | |
| | | | CN102015767 | |
| CORV50 | Light chain variable region, | s231.19 | US20110159001 SEQ ID NO: 88; | 26144 |
| | SARS antibody | | WO2009128963; EP2242768; | |
| | | | CN102015767 | |
| CORV51 | Light chain variable region, | s230.14 + 15 | US20110159001 SEQ ID NO: 92; | 26145 |
| | SARS antibody | | WO2009128963; EP2242768; | |
| | | | CN102015767 | |
| CORV52 | Light chain variable region, | s227.14 | US20110159001 SEQ ID NO: 96; | 26146 |
| | SARS antibody | | WO2009128963; EP2242768; | |
| | | | CN102015767 | |
| CORV53 | Light chain variable region, | s109.8 | US20110159001 SEQ ID NO: 101; | 26147 |
| | SARS antibody | | WO2009128963; EP2242768; | |
| | | | CN102015767 | |
| CORV54 | Light chain variable region, | Fab58 | CN1513874 | 26148 |
| | SARS antibody | | | |
| CORV55 | Light chain variable region, | Fab59 | CN1513874 | 26149 |
| | SARS antibody | | | |
| CORN56 | Light chain variable region, | 3C7 | U.S. Pat. No. 7,728,110 SEQ | 26150 |
| | SARS human monoclonal | | ID NO: 58; WO2008060331; | |
| | antibody | | EP2035454A2, US20080248043 | |
| CORV57 | Light chain variable region, | F26G18 | U.S. Pat. No. 7,622,112 SEQ | 26151 |
| | SARS human monoclonal | | ID NO: 14; WO2005054469; | |
| | antibody | | US20080248043; US20080081047 | |
| CORV58 | Light chain, Humanized | | CN103864924 SEQ ID NO: 4 | 26152 |
| | neutralizing murine monoclonal | | | |
| | MERS | | | |
| CORV59 | Light chain, MERS | m336 | WO2015057942 SEQ ID NO: 2 | 26153 |
| CORV60 | Light chain, MERS | m337 | WO2015057942 SEQ ID NO: 10 | 26154 |
| CORV61 | Light chain, MERS | m338 | WO2015057942 SEQ ID NO: 17 | 26155 |
| CORV62 | Light chain, MERS | 2E 6 | CN104447986 SEQ ID NO: 4 | 26156 |
| CORV63 | Light chain, MERS | M336 | Ying et al., Nat Commun 6, 8223 | 26157 |
| | | | (2015), NCBI Accession # | |
| | | | 4XAK_L (214aa) | |
| CORV64 | Variable heavy chain-constant | 4C2Fab | CN103864924 SEQ ID NO: 7 | 26158 |
| | heavy chain 1, Humanized | | | |
| | neutralizing murine monoclonal | | | |
| | MERS | | | |
| CORV65 | Variable light chain-constant | 4C2Fab | CN103864924 SEQ ID NO: 9 | 26159 |
| | light chain 1, Humanized | | | |
| | neutralizing murine monoclonal | | | |
| | MERS | | | |

In some embodiments, the payload region of the viral particle comprises one or more nucleic acid sequences, fragments or variants thereof or encodes one or more polypeptides, fragments or variants thereof described in U.S. Pat. No. 7,629,443, US Publication No. US20080254440, Chinese Publication No. CN103613666, CN1570638, CN101522208, CN1673231, CN1590409, CN1557838, and CN1488645, the contents of each of which are herein incorporated by reference in their entirety, against SARS.

In some embodiments, the payload region of the viral particle comprises one or more nucleic acid sequences encoding one or more of the payload antibody polypeptides listed in Table 37 against John Cunningham Vitus (JCV1-JCA168; SEQ ID NO: 26160-26223).

TABLE 37
Antibodies against John Cunningham Virus

| Antibody | | Antibody | Reference | SEQ ID |
|---|---|---|---|---|
| No. | Description | Name | Information | NO |
| JCV1 | Heavy chain | 14G8 | US20150056188 SEQ ID NO: 1 | 26160 |
| JCV2 | Heavy chain | 16H5 | US20150056188 SEQ ID NO: 5 | 26161 |
| JCV3 | Heavy chain | 18C9 | US20150056188 SEQ ID NO: 9 | 26162 |
| JCV4 | Heavy chain | 34C6 | US20150056188 SEQ ID NO: 13 | 26163 |
| JCV5 | Heavy chain | 18C9 N55S | US20150056188 SEQ ID NO: 16 | 26164 |
| JCV6 | Heavy chain | 18C9 N55Q | US20150056188 SEQ ID NO: 18 | 26165 |
| JCV7 | Heavy chain | 18C9 N55D | US20150056188 SEQ ID NO: 20 | 26166 |
| JCV8 | Heavy chain | 18C9 N55H | US20150056188 SEQ ID NO: 22 | 26167 |
| JCV9 | Heavy chain | 18C9 N55T | US20150056188 SEQ ID NO: 24 | 26168 |
| JCV10 | Heavy chain | 18C9 N55A | US20150056188 SEQ ID NO: 26 | 26169 |
| JCV11 | Heavy chain | 18C9 N55L | US20150056188 SEQ ID NO: 28 | 26170 |
| JCV12 | Heavy chain | 18C9 N55X | US20150056188 SEQ ID NO: 30 | 26171 |
| JCV13 | Heavy chain | 18C9 G56A | US20150056188 SEQ ID NO: 32 | 26172 |
| JCV14 | Heavy chain | 18C9 G56V | US20150056188 SEQ ID NO: 34 | 26173 |
| JCV15 | Heavy chain | 18C9 G56P | US20150056188 SEQ ID NO: 36 | 26174 |
| JCV16 | Heavy chain | 18C9 G56X | US20150056188 SEQ ID NO: 38 | 26175 |
| JCV17 | Heavy chain | 399-h (C35A | US20150050271 SEQ ID NO: 20 | 26176 |
| | | V50A) | | |
| JCV18 | Heavy chain | Antibody from | US20150050271 SEQ ID NO: 66 | 26177 |
| | | US20150050271 | | |
| JCV19 | Heavy chain | H0 | US20150050271 SEQ ID NO: 51 | 26178 |
| JCV20 | Heavy chain | H1 | US20150050271 SEQ ID NO: 52 | 26179 |
| JCV21 | Heavy chain | H3 | US20150050271 SEQ ID NO: 54 | 26180 |
| JCV22 | Heavy chain | H4 | US20150050271 SEQ ID NO: 55 | 26181 |
| JCV23 | Heavy chain | H5 | US20150050271 SEQ ID NO: 56 | 26182 |
| JCV24 | Heavy chain | H6 | US20150050271 SEQ ID NO: 57 | 26183 |
| JCV25 | Heavy chain | H7 | US20150050271 SEQ ID NO: 58 | 26184 |
| JCV26 | Heavy chain | H8 | US20150050271 SEQ ID NO: 59 | 26185 |
| JCV27 | Heavy chain | H9 | US20150050271 SEQ ID NO: 60 | 26186 |
| JCV28 | Heavy chain | L0 | US20150050271 SEQ ID NO: 48 | 26187 |
| JCV29 | Heavy chain | jcv411_vh | US20150050271 SEQ ID NO: 43 | 26188 |
| JCV30 | Heavy chain | IGHV3-30-3x01 | US20150050271 SEQ ID NO: 44 | 26189 |
| JCV31 | Heavy chain | H0 | US20150050271 SEQ ID NO: 19 | 26190 |
| JCV32 | Heavy chain | H0 V50G | US20150050271 SEQ ID NO: 21 | 26191 |
| JCV33 | Heavy chain | H1 | US20150050271 SEQ ID NO: 22 | 26192 |
| JCV34 | Heavy chain | H2 | US20150050271 SEQ ID NO: 23 | 26193 |
| JCV35 | Heavy chain | H3 | US20150050271 SEQ ID NO: 24 | 26194 |
| JCV36 | Heavy chain | H4 | US20150050271 SEQ ID NO: 25 | 26195 |
| JCV37 | Heavy chain | H5 | US20150050271 SEQ ID NO: 26 | 26196 |
| JCV38 | Heavy chain | H6 | US20150050271 SEQ ID NO: 27 | 26197 |
| JCV39 | Heavy chain | H7 | US20150050271 SEQ ID NO: 28 | 26198 |
| JCV40 | Heavy chain | H8 | US20150050271 SEQ ID NO: 29 | 26199 |
| JCV41 | Heavy chain | H9 | US20150050271 SEQ ID NO: 30 | 26200 |
| JCV42 | Heavy chain | GRE1 | US20150191530 SEQ ID NO: 1 | 26201 |
| | variable region | | | |
| JCV43 | Heavy chain | R399 | US20150050271 SEQ ID NO: 6 | 26202 |
| | variable region | | | |
| JCV44 | Light chain | 14G8 | US20150056188 SEQ ID NO: 3 | 26203 |
| JCV45 | Light chain | 16H5 | US20150056188 SEQ ID NO: 7 | 26204 |
| JCV46 | Light chain | 18C9 | US20150056188 SEQ ID NO: 11 | 26205 |
| JCV47 | Light chain | 34C6 | US20150056188 SEQ ID NO: 14 | 26206 |
| JCV48 | Light chain | 18C9 C96L | US20150056188 SEQ ID NO: 40 | 26207 |
| JCV49 | Light chain | 18C9 C96S | US20150056188 SEQ ID NO: 42 | 26208 |
| JCV50 | Light chain | 18C9 C96A | US20150056188 SEQ ID NO: 44 | 26209 |
| JCV51 | Light chain | 18C9 C96X | US20150056188 SEQ ID NO: 46 | 26210 |
| JCV52 | Light chain | 399-1 (N31G), L | US20150050271 SEQ ID NO: 15 | 26211 |
| JCV53 | Light chain | Antibody from | US20150050271 SEQ ID NO: 67 | 26212 |
| | | US20150050271 | | |
| JCV54 | Light chain | H2 | US20150050271 SEQ ID NO: 53 | 26213 |
| JCV55 | Light chain | L1 | US20150050271 SEQ ID NO: 49 | 26214 |
| JCV56 | Light chain | L2 | US20150050271 SEQ ID NO: 50 | 26215 |
| JCV57 | Light chain | IGKV1D-13x01 | US20150050271 SEQ ID NO: 39 | 26216 |
| JCV58 | Light chain | L0 | US20150050271 SEQ ID NO: 11 | 26217 |
| JCV59 | Light chain | L1 | US20150050271 SEQ ID NO: 12 | 26218 |
| JCV60 | Light chain | L2 | US20150050271 SEQ ID NO: 13 | 26219 |
| JCV61 | Light chain | L2 N31A | US20150050271 SEQ ID NO: 14 | 26220 |
| JCV62 | Light chain | GRE1 | US20150191530 SEQ ID NO: 2 | 26221 |
| | variable region | | | |
| JCV63 | Light chain | R399 | US20150050271 SEQ ID NO: 1 | 26222 |
| | variable region | | | |
| JCV64 | Light chain | R411, jcv411_vh | US20150050271 SEQ ID NO: 38 | 26223 |
| | variable region | | | |

In some embodiments, the payload region of the viral particle comprises one or more nucleic acid sequences encoding one or more of the payload antibody polypeptides listed in Table 38 against Poxvirus (POXV1-POXV10; SEQ ID NO: 26224-26233).

TABLE 38
Antibodies against Poxvirus

| Antibody | | Antibody | Reference | SEQ ID |
|---|---|---|---|---|
| No. | Description | Name | Information | NO |
| POXV1 | Heavy chain | B5R | U.S. Pat. No. | 26224 |
| | variable region, | binding | 8,623,370 | |
| | B5R envelope | antibody | SEQ ID NO: 2 | |
| | protein | | | |
| POXV2 | Heavy chain | B5R | U.S. Pat. No. | 26225 |
| | variable region, | binding | 8,623,370 | |
| | B5R envelope | antibody | SEQ ID NO: 6 | |
| | protein | | | |
| POXV3 | Heavy chain | B5R | U.S. Pat. No. | 26226 |
| | variable region, | binding | 8,623,370 | |
| | B5R envelope | antibody | SEQ ID NO: 10 | |
| | protein | | | |
| POXV4 | Heavy chain | B5R | U.S. Pat. No. | 26227 |
| | variable region, | binding | 8,623,370 | |
| | B5R envelope | antibody | SEQ ID NO: 14 | |
| | protein | | | |
| POXV5 | Heavy chain, | H3L | US20140186370 | 26228 |
| | H3L envelope | binding | SEQ ID NO: 14 | |
| | protein | antibody | | |
| POXV6 | Light chain | B5R | U.S. Pat. No. | 26229 |
| | variable region, | binding | 8,623,370 | |
| | B5R envelope | antibody | SEQ ID NO: 4 | |
| | protein | | | |
| POXV7 | Light chain | B5R | U.S. Pat. No. | 26230 |
| | variable region, | binding | 8,623,370 | |
| | B5R envelope | antibody | SEQ ID NO: 8 | |
| | protein | | | |
| POXV8 | Light chain | B5R | U.S. Pat. No. | 26231 |
| | variable region, | binding | 8,623,370 | |
| | B5R envelope | antibody | SEQ ID NO: 12 | |
| | protein | | | |
| POXV9 | Light chain | B5R | U.S. Pat. No. | 26232 |
| | variable region, | binding | 8,623,370 | |
| | B5R envelope | antibody | SEQ ID NO: 16 | |
| | protein | | | |
| POXV10 | Light chain | H3L | US20140186370 | 26233 |
| | H3L envelope | binding | SEQ ID NO: 16 | |
| | protein | antibody | | |

In some embodiments, the payload region of the viral particle comprises one or more nucleic acid sequences encoding one or more of the payload antibody polypeptides listed in Table 39 against Enterovirus 71 (ENTV1-ENTV16; SEQ ID NO: 26234-26249).

TABLE 39
Antibodies against Enterovirus 71

| Antibody | | Antibody | Reference | SEQ ID |
|---|---|---|---|---|
| No. | Description | Name | Information | NO |
| ENTV1 | Heavy chain | | CN102718864A | 26234 |
| | variable region | | SEQ ID NO: 2 | |
| ENTV2 | Heavy chain | E18 | WO2015092668 | 26235 |
| | variable region | | SEQ ID NO: 1 | |
| ENTV3 | Heavy chain | E19 | WO2015092668 | 26236 |
| | variable region | | SEQ ID NO: 3 | |
| ENTV4 | Heavy chain | E20 | WO2015092668 | 26237 |
| | variable region | | SEQ ID NO: 5 | |
| ENTV5 | Heavy chain | E19 humanized | WO2015092668 | 26238 |
| | variable region | VH1 | SEQ ID NO: 19 | |
| ENTV6 | Heavy chain | E19 humanized | WO2015092668 | 26239 |
| | variable region | VH2 | SEQ ID NO: 20 | |
| ENTV7 | Heavy chain | E19 humanized | WO2015092668 | 26240 |
| | variable region | VH3 | SEQ ID NO: 21 | |
| ENTV8 | Heavy chain | E19 humanized | WO2015092668 | 26241 |
| | variable region | VH4 | SEQ ID NO: 22 | |
| ENTV9 | Light chain | | CN102718864A | 26242 |
| | variable region | | SEQ ID NO: 1 | |
| ENTV10 | Light chain | E18 | WO2015092668 | 26243 |
| | variable region | | SEQ ID NO: 2 | |
| ENTV11 | Light chain | E19 | WO2015092668 | 26244 |
| | variable region | | SEQ ID NO: 4 | |
| ENTV12 | Light chain | E20 | WO2015092668 | 26245 |
| | variable region | | SEQ ID NO: 6 | |
| ENTV13 | Light chain | E18 VL2 | WO2015092668 | 26246 |
| | variable region | | SEQ ID NO: 15 | |
| ENTV14 | Light chain | E19 humanized | WO2015092668 | 26247 |
| | variable region | VL1 | SEQ ID NO: 16 | |
| ENTV15 | Light chain | E19 humanized | WO2015092668 | 26248 |
| | variable region | VL2 | SEQ ID NO: 17 | |
| ENTV16 | Light chain | E19 humanized | WO2015092668 | 26249 |
| | variable region | VL3 | SEQ ID NO: 18 | |

In some embodiments, the payload region of the viral particle comprises one or more nucleic acid sequences, fragments or variants thereof or encodes one or more polypeptides, fragments or variants thereof described in Chinese Publication No, CN104357400, the contents of which are herein incorporated by reference in their entirety, against EV71.

In some embodiments, the payload region of the viral particle comprises one or more nucleic acid sequences, fragments or variants encoding MAB979, fragments or variants thereof for treating a disease and/or disorder or preventing a disease and/or disorder. As a non-limiting example, the disease and/or disorder is EV71.

In some embodiments, the payload region of the viral particle comprises one or more nucleic acid sequences encoding one or more of the payload antibody polypeptides listed in Table 40 against Rubella Virus (RUBV1-RUBV4; SEQ ID NO: 26250-26253).

TABLE 40
Antibodies against Rubella Virus

| Antibody | | Antibody | Reference | SEQ ID |
|---|---|---|---|---|
| No. | Description | Name | Information | NO |
| RUBV1 | Heavy chain | DDF-RuV1 | US20100143376 | 26250 |
| | variable region | | SEQ ID NO: 2 | |
| RUBV2 | Heavy chain | DDF-RuV2 | US20100143376 | 26251 |
| | variable region | | SEQ ID NO: 9 | |
| RUBV3 | Light chain | DDF-RuV1 | US20100143376 | 26252 |
| | variable region | | SEQ ID NO: 7 | |
| RUBV4 | Light chain | DDF-RuV2 | US20100143376 | 26253 |
| | variable region | | SEQ ID NO: 14 | |

In some embodiments, the payload region of the viral particle comprises one or more nucleic acid sequences encoding one or more of the payload antibody polypeptides listed in Table 41 against Human Papilloma Virus (HPV1-HPV2; SEQ ID NO: 6896-6897).

TABLE 41
Antibodies against Human Papilloma Virus

| Antibody | | Reference | SEQ ID |
|---|---|---|---|
| No. | Description | Information | NO |
| HPV1 | Heavy chain | WO2015096269 | 26254 |
| | variable region | SEQ ID NO: 1 | |
| HPV2 | Light chain | WO2015096269 | 26255 |
| | variable region | SEQ ID NO: 2 | |

In some embodiments, the payload region of the viral particle comprises one or more nucleic acid sequences, fragments or variants thereof or encodes one or more polypeptides, fragments or variants thereof described in US Publication No. US20130337438, the contents of which are herein incorporated by reference in their entirety, against HPV.

In some embodiments, the payload region of the viral particle comprises one or more nucleic acid sequences encoding one or more of the broadly neutralizing payload antibody polypeptides listed in Table 42 against viruses (VIR1-VIR14; SEQ ID NO: 26256-26269).

TABLE 42
Broadly Neutralizing Antibodies for Viruses

| Antibody | | Antibody | Reference | SEQ ID |
|---|---|---|---|---|
| No. | Description | Name | Information | NO |
| VIR1 | Heavy chain variable region, hepatitis, influenza, | 3G4 | U.S. Pat. No. | 26256 |
| | HIV, herpes, paramyxovirus, poxvirus, | | 7,611,704 | |
| | rhabdovirus or arenavirus | | SEQ ID NO: 2 | |
| VIR2 | Heavy chain variable region, hepatitis, influenza, | 3G4 | U.S. Pat. No. | 26257 |
| | HIV, herpes, paramyxovirus, poxvirus, | | 7,611,704 | |
| | rhabdovirus or arenavirus | | SEQ ID NO: 4 | |
| VIR3 | Heavy chain variable region, HIV, herpes, | 679 | U.S. Pat. No. | 26258 |
| | cytomegalovirus, rabies, influenza, hepatitis B, | | 7,429,381 | |
| | Sendai, feline leukemia, Reo, polio, human serum | | SEQ ID NO: 4 | |
| | parvo-like, simian 40, respiratory syncytial, | | | |
| | mouse mammary tumor, Varicella-Zoster, light | | | |
| | chain variable region, Dengue, rubella, measles, | | | |
| | adenovirus, human T-cell leukemias, Epstein- | | | |
| | Barr, murine leukemia, mumps, vesicular | | | |
| | stomatitis, Sindbis, lymphocytic | | | |
| | choriomeningitis, wart and blue tongue | | | |
| VIR4 | Heavy chain variable region, HIV, herpes, | Mu-9V | U.S. Pat. No. | 26259 |
| | cytomegalovirus, rabies, influenza, hepatitis B, | | 7,429,381 | |
| | Sendai, feline leukemia, Reo, polio, human serum | | SEQ ID NO: 10 | |
| | parvo-like, simian 40, respiratory syncytial, | | | |
| | mouse mammary tumor, Varicella-Zoster, | | | |
| | Dengue, rubella, measles, adenovirus, human T- | | | |
| | cell leukemias, Epstein-Barr, murine leukemia, | | | |
| | mumps, vesicular stomatitis, Sindbis, | | | |
| | lymphocytic choriomeningitis, wart and blue | | | |
| | tongue, light chain variable region | | | |
| VIR5 | Heavy chain variable region, HIV, herpes, | humanized | U.S. Pat. No. | 26260 |
| | cytomegalovirus, rabies, influenza, hepatitis B, | Mu-9 | 7,429,381 | |
| | Sendai, feline leukemia, Reo, polio, human serum | | SEQ ID NO: 14 | |
| | parvo-like, simian 40, respiratory syncytial, | | | |
| | mouse mammary tumor, Varicella-Zoster, | | | |
| | Dengue, rubella, measles, adenovirus, human T- | | | |
| | cell leukemias, Epstein-Barr, murine leukemia, | | | |
| | mumps, vesicular stomatitis, Sindbis, | | | |
| | lymphocytic choriomeningitis, wart and blue | | | |
| | tongue, light chain variable region | | | |
| VIR6 | Heavy chain variable region, Human | Fab-2 | US20120269801 | 26261 |
| | cytomegalovirus, HCMV, human T-cell leukemia | Clone3 | SEQ ID NO: 6 | |
| | virus type 1, HIV-1, simian immunodeficiency | | | |
| | virus, Ebola virus, Herpesvirus saimiri virus, | | | |
| | influenza virus, and vaccinia virus | | | |
| VIR7 | Heavy chain variable region, Human | Fab-3 | US20120269801 | 26262 |
| | cytomegalovirus, HCMV, human T-cell leukemia | Clone 7 | SEQ ID NO: 10 | |
| | virus type 1, HIV-1, simian immunodeficiency | | | |
| | virus, Ebola virus, Herpesvirus saimiri virus, | | | |
| | influenza virus, and vaccinia virus | | | |
| VIR8 | Light chain variable region, HIV, herpes, | Mu-9V | U.S. Pat. No. | 26263 |
| | cytomegalovirus, rabies, influenza, hepatitis B, | | 7,429,381 | |
| | Sendai, feline leukemia, Reo, polio, human serum | | SEQ ID NO: 8 | |
| | parvo-like, simian 40, respiratory syncytial, | | | |
| | mouse mammary tumor, Varicella-Zoster, | | | |
| | Dengue, rubella, measles, adenovirus, human T- | | | |
| | cell leukemias, Epstein-Barr, murine leukemia, | | | |
| | mumps, vesicular stomatitis, Sindbis, | | | |
| | lymphocytic choriomeningitis, wart and blue | | | |
| | tongue, light chain variable region | | | |
| VIR9 | Light chain variable region, HIV, herpes, | humanized | U.S. Pat. No. | 26264 |
| | cytomegalovirus, rabies, influenza, hepatitis B, | Mu-9 | 7,429,381 | |
| | Sendai, feline leukemia, Reo, polio, human serum | | SEQ ID NO: 12 | |
| | parvo-like, simian 40, respiratory syncytial, | | | |
| | mouse mammary tumor, Varicella-Zoster, | | | |
| | Dengue, rubella, measles, adenovirus, human T- | | | |
| | cell leukemias, Epstein-Barr, murine leukemia, | | | |
| | mumps, vesicular stomatitis, Sindbis, | | | |
| | lymphocytic choriomeningitis, wart and blue | | | |
| | tongue, light chain variable region | | | |
| VIR10 | Light chain variable region, HIV, herpes, | 679 | U.S. Pat. No. | 26265 |
| | cytomegalovirus, rabies, influenza, hepatitis B, | | 7,429,381 | |
| | Sendai, feline leukemia, Reo, polio, human serum | | SEQ ID NO: 2 | |
| | parvo-like, simian 40, respiratory syncytial, | | | |
| | mouse mammary tumor, Varicella-Zoster, | | | |
| | Dengue, rubella, measles, adenovirus, human T- | | | |
| | cell leukemias, Epstein-Barr, murine leukemia, | | | |
| | mumps, vesicular stomatitis, Sindbis, | | | |
| | lymphocytic choriomeningitis, wart and blue | | | |
| | tongue | | | |
| VIR11 | Light chain variable region, Human | Fab-3 | US20120269801 | 26266 |
| | cytomegalovirus, HCMV, human T-cell leukemia | Clone 7 | SEQ ID NO: 8 | |
| | virus type 1, HIV-1, simian immunodeficiency | | | |
| | virus, Ebola virus, Herpesvirus saimiri virus, | | | |
| | influenza virus, and vaccinia virus | | | |
| VIR12 | Light chain variable, Human cytomegalovirus, | Fab-2 | US20120269801 | 26267 |
| | HCMV, human T-cell leukemia virus type 1, | Clone3 | SEQ ID NO: 4 | |
| | HIV-1, simian immunodeficiency virus, Ebola | | | |
| | virus, Herpesvirus saimiri virus, influenza virus, | | | |
| | and vaccinia virus, region | | | |
| VIR13 | ScFv, hepatitis, influenza, HIV, herpes, | 3A2 | U.S. Pat. No. | 26268 |
| | paramyxovirus, poxvirus, rhabdovirus or | | 7,611,704 | |
| | arenavirus | | SEQ ID NO: 6 | |
| VIR14 | ScFv, HIV, herpes, cytomegalovirus, rabies, | 679 | U.S. Pat. No. | 26269 |
| | influenza, hepatitis B, Sendai, feline leukemia, | | 7,429,381 | |
| | Reo, polio, human serum parvo-like, simian 40, | | SEQ ID NO: 6 | |
| | respiratory syncytial, mouse mammary tumor, | | | |
| | Varicella-Zoster, Dengue, rubella, measles, | | | |
| | adenovirus, human T-cell leukemias, Epstein- | | | |
| | Barr, murine leukemia, mumps, vesicular | | | |
| | stomatitis, Sindbis, lymphocytic | | | |
| | choriomeningitis, wart and blue tongue | | | |

In some embodiments, the payload region of the viral particle comprises one or more nucleic acid sequences encoding one or more of the payload antibody polypeptides listed in Table 43 against Pseudomonas Aeruginosa (PSEU1-PSEU285; SEQ ID NO: 26270-26554).

TABLE 43
Antibodies against Pseudomonas Aeruginosa

| Antibody | | Antibody | Reference | SEQ |
|---|---|---|---|---|
| No. | Description | Name | Information | ID NO |
| PSEU1 | Bivalent nanobody | 260 (1E11-40GS-2B10) | US20150044215 SEQ ID NO: 118 | 26270 |
| PSEU2 | Bivalent nanobody | 272 (11B09-40GS-10C05) | US20150044215 SEQ ID NO: 119 | 26271 |
| PSEU3 | Bivalent nanobody | 308 (6B05-40GS-1E11) | US20150044215 SEQ ID NO: 120 | 26272 |
| PSEU4 | Bivalent nanobody | 264 (1E11-40GS-2B02) | US20150044215 SEQ ID NO: 121 | 26273 |
| PSEU5 | Bivalent nanobody | 302 (5H01-40GS-7C10) | US20150044215 SEQ ID NO: 122 | 26274 |
| PSEU6 | Bivalent nanobody | 234 (7C10-40GS-5H01) | US20150044215 SEQ ID NO: 123 | 26275 |
| PSEU7 | Bivalent nanobody | 064 (13F07-40GS-7C10) | US20150044215 SEQ ID NO: 124 | 26276 |
| PSEU8 | Bivalent nanobody | 275 (2G09-40GC-5H10) | US20150044215 SEQ ID NO: 125 | 26277 |
| PSEU9 | Bivalent nanobody | 083 (7C10-40GS-11B09) | US20150044215 SEQ ID NO: 126 | 26278 |
| PSEU10 | Bivalent nanobody | 087 (1E11-40GS-7C10) | US20150044215 SEQ ID NO: 127 | 26279 |
| PSEU11 | Bivalent nanobody | 269 (6B05-40GS-13F07) | US20150044215 SEQ ID NO: 128 | 26280 |
| PSEU12 | Bivalent nanobody | 256 (13F07-40GS-5H01) | US20150044215 SEQ ID NO: 129 | 26281 |
| PSEU13 | Bivalent nanobody | 277 (5H01-40GS-11B09) | US20150044215 SEQ ID NO: 130 | 26282 |
| PSEU14 | Bivalent nanobody | 257 (13F07-40GS-2B10) | US20150044215 SEQ ID NO: 131 | 26283 |
| PSEU15 | Bivalent nanobody | 285 (13F07-40GS-2B02) | US20150044215 SEQ ID NO: 132 | 26284 |
| PSEU16 | Bivalent nanobody | 115 (11B09-40GS-13F07) | US20150044215 SEQ ID NO: 133 | 26285 |
| PSEU17 | Bivalent nanobody | 258 (13F07-40GS-14E10) | US20150044215 SEQ ID NO: 134 | 26286 |
| PSEU18 | Bivalent nanobody | 283 (7E09-40G5-6B05) | US20150044215 SEQ ID NO: 135 | 26287 |
| PSEU19 | Bivalent nanobody | 271 (7C10-40GS-14E10) | US20150044215 SEQ ID NO: 136 | 26288 |
| PSEU20 | Bivalent nanobody | 259 (1E11-40GS-5H01) | US20150044215 SEQ ID NO: 137 | 26289 |
| PSEU21 | Bivalent nanobody | 319 (13F07-40GS-6B05) | US20150044215 SEQ ID NO: 138 | 26290 |
| PSEU22 | Bivalent nanobody | 335 (5H01-40G5-1E11) | US20150044215 SEQ ID NO: 139 | 26291 |
| PSEU23 | Bivalent nanobody | 261 (5H01-40GS-2B10) | US20150044215 SEQ ID NO: 140 | 26292 |
| PSEU24 | Bivalent nanobody | 262 (7E09-40GS-7C10) | US20150044215 SEQ ID NO: 141 | 26293 |
| PSEU25 | Constant heavy chain | | US20150044215 SEQ ID NO: 148 | 26294 |
| PSEU26 | Constant light chain | | US20150044215 SEQ ID NO: 149 | 26295 |
| PSEU27 | Heavy chain | Panobacumab | U.S. Pat. No. 8,197,816 SEQ ID NO: 8 | 26296 |
| PSEU28 | Heavy chain | | US20130156696 SEQ ID NO: 2 | 26297 |
| PSEU29 | Heavy chain | | U.S. Pat. No. 7,494,653 SEQ ID NO: 2 | 26298 |
| PSEU30 | Heavy chain | KB0001 | U.S. Pat. No. 8,044,181 SEQ ID NO: 3 | 26299 |
| | variable region | | | |
| PSEU31 | Heavy chain | KB0001 | U.S. Pat. No. 8,044,181 SEQ ID NO: 5 | 26300 |
| | variable region | | | |
| PSEU32 | Heavy chain | KB0001 | U.S. Pat. No. 8,044,181 SEQ ID NO: 7 | 26301 |
| | variable region | | | |
| PSEU33 | Heavy chain | KB0001 | U.S. Pat. No. 8,044,181 SEQ ID NO: 9 | 26302 |
| | variable region | | | |
| PSEU34 | Heavy chain | KB0001 | U.S. Pat. No. 8,044,181 SEQ ID NO: 11 | 26303 |
| | variable region | | | |
| PSEU35 | Heavy chain | 1F3 | U.S. Pat. No. 9,085,611 SEQ ID NO: 11 | 26304 |
| | variable region | | | |
| PSEU36 | Heavy chain | 2A4 | U.S. Pat. No. 9,085,611 SEQ ID NO: 13 | 26305 |
| | variable region | | | |
| PSEU37 | Heavy chain | | U.S. Pat. No. 9,085,611 SEQ ID NO: 27 | 26306 |
| | variable region | | | |
| PSEU38 | Heavy chain | mAbs LST-001 | U.S. Pat. No. 8,653,242 SEQ ID NO: 29 | 26307 |
| | variable region | | | |
| PSEU39 | Heavy chain | rnAbs LST-002 | U.S. Pat. No. 8,653,242 SEQ ID NO: 49 | 26308 |
| | variable region | | | |
| PSEU40 | Heavy chain | mAbs LST-005 | U.S. Pat. No. 8,653,242 SEQ ID NO: 52 | 26309 |
| | variable region | | | |
| PSEU41 | Heavy chain | rnAbs LST-006 | U.S. Pat. No. 8,653,242 SEQ ID NO: 54 | 26310 |
| | variable region | | | |
| PSEU42 | Heavy chain | mAbs LST-007 | U.S. Pat. No. 8,653,242 SEQ ID NO: 13 | 26311 |
| | variable region | | | |
| PSEU43 | Heavy chain | mAbs LST-008 | U.S. Pat. No. 8,653,242 SEQ ID NO: 15 | 26312 |
| | variable region | | | |
| PSEU44 | Heavy chain | 310BO6 | U.S. Pat. No. 7,597,893 SEQ ID NO: 8 | 26313 |
| | variable region | | | |
| PSEU45 | Heavy chain | Cam-003 | US20140227285 SEQ ID NO: 1 | 26314 |
| | variable region | | | |
| PSEU46 | Heavy chain | Cam-004 | US20140227285 SEQ ID NO: 3 | 26315 |
| | variable region | | | |
| PSEU47 | Heavy chain | Cam-005 | US20140227285 SEQ ID NO: 4 | 26316 |
| | variable region | | | |
| PSEU48 | Heavy chain | WapR-001 | US20140227285 SEQ ID NO: 5 | 26317 |
| | variable region | | | |
| PSEU49 | Heavy chain | WapR-002 | US20140227285 SEQ ID NO: 7 | 26318 |
| | variable region | | | |
| PSEU50 | Heavy chain | WapR-003 | US20140227285 SEQ ID NO: 9 | 26319 |
| | variable region | | | |
| PSEU51 | Heavy chain | WapR-004 | US20140227285 SEQ ID NO: 11 | 26320 |
| | variable region | | | |
| PSEU52 | Heavy chain | WapR-007 | US20140227285 SEQ ID NO: 13 | 26321 |
| | variable region | | | |
| PSEU53 | Heavy chain | WapR-016 | US20140227285 SEQ ID NO: 15 | 26322 |
| | variable region | | | |
| PSEU54 | Heavy chain | 1584 | US20130045207 SEQ ID NO: 8 | 26323 |
| | variable region | | | |
| PSEU55 | Heavy chain | 1573 | US20130045207 SEQ ID NO: 16 | 26324 |
| | variable region | | | |
| PSEU56 | Heavy chain | 1572 | US20130045207 SEQ ID NO: 24 | 26325 |
| | variable region | | | |
| PSEU57 | Heavy chain | 1587 | US20130045207 SEQ ID NO: 32 | 26326 |
| | variable region | | | |
| PSEU58 | Heavy chain | 3099 | US20130022604 SEQ ID NO: 8 | 26327 |
| | variable region | | | |
| PSEU59 | Heavy chain | 2745 | US20130022604 SEQ ID NO: 16 | 26328 |
| | variable region | | | |
| PSEU60 | Heavy chain | 2459 | US20130022604 SEQ ID NO: 24 | 26329 |
| | variable region | | | |
| PSEU61 | Heavy chain | 2316 | US20130022606 SEQ ID NO: 8 | 26330 |
| | variable region | | | |
| PSEU62 | Heavy chain | 1838 | US20130022606 SEQ ID NO: 16 | 26331 |
| | variable region | | | |
| PSEU63 | Heavy chain | 2314 | US20130022606 SEQ ID NO: 24 | 26332 |
| | variable region | | | |
| PSEU64 | Heavy chain | 2326 | US20130022606 SEQ ID NO: 32 | 26333 |
| | variable region | | | |
| PSEU65 | Heavy chain | 2328 | US20130022606 SEQ ID NO: 40 | 26334 |
| | variable region | | | |
| PSEU66 | Heavy chain | 2438 | US20130022606 SEQ ID NO: 48 | 26335 |
| | variable region | | | |
| PSEU67 | Heavy chain | 1774 | US20130004500 SEQ ID NO: 8 | 26336 |
| | variable region | | | |
| PSEU68 | Heavy chain | 1660 | US20130004500 SEQ ID NO: 16 | 26337 |
| | variable region | | | |
| PSEU69 | Heavy chain | 1923 | US20130004500 SEQ ID NO: 24 | 26338 |
| | variable region | | | |
| PSEU70 | Heavy chain | 1656 | US20130004499 SEQ ID NO: 8 | 26339 |
| | variable region | | | |
| PSEU71 | Heavy chain | 1640 | US20130004499 SEQ ID NO: 16 | 26340 |
| | variable region | | | |
| PSEU72 | Heavy chain | 2459 | US20130004499 SEQ ID NO: 24 | 26341 |
| | variable region | | | |
| PSEU73 | Heavy chain | | US20120114657 SEQ ID NO: 8 | 26342 |
| | variable region | | | |
| PSEU74 | Heavy chain | Anti-It-2 | US20110177087 SEQ ID NO: 13 | 26343 |
| | variable region | | | |
| PSEU75 | Heavy chain | Anti-It-3 | US20110177087 SEQ ID NO: 14 | 26344 |
| | variable region | | | |
| PSEU76 | Heavy chain | Anti-It-4 | US20110177087 SEQ ID NO: 15 | 26345 |
| | variable region | | | |
| PSEU77 | Heavy chain | Anti-It-5 | US20110177087 SEQ ID NO: 16 | 26346 |
| | variable region | | | |
| PSEU78 | Heavy chain | Anti-It-6 | US20110177087 SEQ ID NO: 17 | 26347 |
| | variable region | | | |
| PSEU79 | Heavy chain | Anti-170003 | US20110177087 SEQ ID NO: 18 | 26348 |
| | variable region | | | |
| PSEU80 | Heavy chain | Anti-170006 | US20110177087 SEQ ID NO: 19 | 26349 |
| | variable region | | | |
| PSEU81 | Heavy chain | Anti-Pa01 | US20110177087 SEQ ID NO: 20 | 26350 |
| | variable region | | | |
| PSEU82 | Heavy chain | Anti-IATS016 | US20110177087 SEQ ID NO: 21 | 26351 |
| | variable region | | | |
| PSEU83 | Heavy chain | | US20090191186 SEQ ID NO: 1 | 26352 |
| | variable region | | | |
| PSEU84 | Heavy chain | | US20090191186 SEQ ID NO: 11 | 26353 |
| | variable region | | | |
| PSEU85 | Heavy chain | | US20090191186 SEQ ID NO: 3 | 26354 |
| | variable region | | | |
| PSEU86 | Heavy chain | | US20090191186 SEQ ID NO: 7 | 26355 |
| | variable region | | | |
| PSEU87 | Heavy chain | | US20090191186 SEQ ID NO: 9 | 26356 |
| | variable region | | | |
| PSEU88 | Heavy chain | | US20090191186 SEQ ID NO: 5 | 26357 |
| | variable region | | | |
| PSEU89 | Heavy chain | | US20090191186 SEQ ID NO: 13 | 26358 |
| | variable region | | | |
| PSEU90 | Heavy chain | | US20090191186 SEQ ID NO: 21 | 26359 |
| | variable region | | | |
| PSEU91 | Heavy chain | | US20090191186 SEQ ID NO: 17 | 26360 |
| | variable region | | | |
| PSEU92 | Heavy chain | | US20090191186 SEQ ID NO: 26 | 26361 |
| | variable region | | | |
| PSEU93 | Heavy chain | | US20090191186 SEQ ID NO: 25 | 26362 |
| | variable region | | | |
| PSEU94 | Heavy chain | | US20090191186 SEQ ID NO: 23 | 26363 |
| | variable region | | | |
| PSEU95 | Heavy chain | | US20090191186 SEQ ID NO: 29 | 26364 |
| | variable region | | | |
| PSEU96 | Heavy chain | | US20090191186 SEQ ID NO: 35 | 26365 |
| | variable region | | | |
| PSEU97 | Heavy chain | V2L2 | WO2014074528 SEQ ID NO: 216 | 26366 |
| | variable region | | | |
| PSEU98 | Heavy chain | V2L2-MD | WO2014074528 SEQ ID NO: 255 | 26367 |
| | variable region | | | |
| PSEU99 | Heavy chain | V2L2-MD and V2L2- | WO2014074528 SEQ ID NO: 256 | 26368 |
| | variable region | GL | | |
| PSEU100 | Heavy chain | V2L2-GL | WO2014074528 SEQ ID NO: 257 | 26369 |
| | variable region | | | |
| PSEU101 | Heavy chain | 2409 | WO2013024905 SEQ ID NO: 16 | 26370 |
| | variable region | | | |
| PSEU102 | Heavy chain | 2453 | WO2013024905 SEQ ID NO: 24 | 26371 |
| | variable region | | | |
| PSEU103 | Heavy chain | S20 | U.S. Pat. No. 7,972,845 SEQ ID NO: 2 | 26372 |
| | variable region | | | |
| PSEU104 | Heavy chain | Fab 13.37 | US20150044215 SEQ ID NO: 142 | 26373 |
| | variable region | | | |
| PSEU105 | Heavy chain | Fab 26.24 | US20150044215 SEQ ID NO: 144 | 26374 |
| | variable region | | | |
| PSEU106 | Heavy chain | Fab 35.36 | US20150044215 SEQ ID NO: 146 | 26375 |
| | variable region | | | |
| PSEU107 | Heavy chain | KB0001 | U.S. Pat. No. 8,044,181 SEQ ID NO: 1 | 26376 |
| | variable region | | | |
| PSEU108 | Heavy chain, LPS | | Horn, M. P. et al. “Preclinical In | 26377 |
| | serotype IATS-O11, | | Vitro and In Vivo characterization | |
| | | | of the fully human monoclonal | |
| | | | IgM antibody KBPA101 specific | |
| | | | for Pseudomonas aeruginosa | |
| | | | serotype IATS-O11”, Antimicrob. | |
| | | | Agents Chemother. 54 (6), 2338- | |
| | | | 2344 (2010) | |
| PSEU109 | J chain | Panobacumab | | 26378 |
| PSEU110 | Light chain | Panobacumab | U.S. Pat. No. 8,197,816 SEQ ID NO: 7 | 26379 |
| PSEU111 | Light chain | | US20130156696 SEQ ID NO: 4 | 26380 |
| PSEU112 | Light chain | | U.S. Pat. No. 7,494,653 SEQ ID NO: 4 | 26381 |
| PSEU113 | Light chain | 1F3 | U.S. Pat. No. 9,085,611 SEQ ID NO: 12 | 26382 |
| | variable region | | | |
| PSEU114 | Light chain | 2A4 | U.S. Pat. No. 9,085,611 SEQ ID NO: 4 | 26383 |
| | variable region | | | |
| PSEU115 | Light chain | | U.S. Pat. No. 9,085,611 SEQ ID NO: 28 | 26384 |
| | variable region | | | |
| PSEU116 | Light chain | mAbs LST-001 | U.S. Pat. No. 8,653,242 SEQ ID NO: 18 | 26385 |
| | variable region | | | |
| PSEU117 | Light chain | mAbs LST-006 | U.S. Pat. No. 8,653,242 SEQ ID NO: 53 | 26386 |
| | variable region | | | |
| PSEU118 | Light chain | mAbs LST-008 | U.S. Pat. No. 8,653,242 SEQ ID NO: 14 | 26387 |
| | variable region | | | |
| PSEU119 | Light chain | mAbs LST-008 | U.S. Pat. No. 8,653,242 SEQ ID NO: 16 | 26388 |
| | variable region | | | |
| PSEU120 | Light chain | 310BO6 | U.S. Pat. No. 7,597,893 SEQ ID NO: 7 | 26389 |
| | variable region | | | |
| PSEU121 | Light chain | Cam-003, Cam-004, | US20140227285 SEQ ID NO: 2 | 26390 |
| | variable region | Cam-005 | | |
| PSEU122 | Light chain | WapR-001 | US20140227285 SEQ ID NO: 6 | 26391 |
| | variable region | | | |
| PSEU123 | Light chain | WapR-002 | US20140227285 SEQ ID NO: 8 | 26392 |
| | variable region | | | |
| PSEU124 | Light chain | WapR-003 | US20140227285 SEQ ID NO: 10 | 26393 |
| | variable region | | | |
| PSEU125 | Light chain | WapR-004, WapR- | US20140227285 SEQ ID NO: 12 | 26394 |
| | variable region | 004RAD | | |
| PSEU126 | Light chain | WapR-007 | US20140227285 SEQ ID NO: 14 | 26395 |
| | variable region | | | |
| PSEU127 | Light chain | WapR-016 | US20140227285 SEQ ID NO: 16 | 26396 |
| | variable region | | | |
| PSEU128 | Light chain | 1584 | US20130045207 SEQ ID NO: 7 | 26397 |
| | variable region | | | |
| PSEU129 | Light chain | 1573 | US20130045207 SEQ ID NO: 15 | 26398 |
| | variable region | | | |
| PSEU130 | Light chain | 1572 | US20130045207 SEQ ID NO: 23 | 26399 |
| | variable region | | | |
| PSEU131 | Light chain | 1587 | US20130045207 SEQ ID NO: 31 | 26400 |
| | variable region | | | |
| PSEU132 | Light chain | 3099 | US20130022604 SEQ ID NO: 7 | 26401 |
| | variable region | | | |
| PSEU133 | Light chain | 2745 | US20130022604 SEQ ID NO: 15 | 26402 |
| | variable region | | | |
| PSEU134 | Light chain | 2459 | US20130022604 SEQ ID NO: 23 | 26403 |
| | variable region | | | |
| PSEU135 | Light chain | 2316 | US20130022606 SEQ ID NO: 7 | 26404 |
| | variable region | | | |
| PSEU136 | Light chain | 1838 | US20130022606 SEQ ID NO: 15 | 26405 |
| | variable region | | | |
| PSEU137 | Light chain | 2314 | US20130022606 SEQ ID NO: 23 | 26406 |
| | variable region | | | |
| PSEU138 | Light chain | 2326 | US20130022606 SEQ ID NO: 31 | 26407 |
| | variable region | | | |
| PSEU139 | Light chain | 2328 | US20130022606 SEQ ID NO: 39 | 26408 |
| | variable region | | | |
| PSEU140 | Light chain | 2438 | US20130022606 SEQ ID NO: 47 | 26409 |
| | variable region | | | |
| PSEU141 | Light chain | 1774 | US20130004500 SEQ ID NO: 7 | 26410 |
| | variable region | | | |
| PSEU142 | Light chain | 1660 | US20130004500 SEQ ID NO: 15 | 26411 |
| | variable region | | | |
| PSEU143 | Light chain | 1923 | US20130004500 SEQ ID NO: 23 | 26412 |
| | variable region | | | |
| PSEU144 | Light chain | 1656 | US20130004499 SEQ ID NO: 7 | 26413 |
| | variable region | | | |
| PSEU145 | Light chain | 1640 | US20130004499 SEQ ID NO: 15 | 26414 |
| | variable region | | | |
| PSEU146 | Light chain | 2459 | US20130004499 SEQ ID NO: 23 | 26415 |
| | variable region | | | |
| PSEU147 | Light chain | | US20120114657 SEQ ID NO: 7 | 26416 |
| | variable region | | | |
| PSEU148 | Light chain | Anti-It-2 | US20110177087 SEQ ID NO: 22 | 26417 |
| | variable region | | | |
| PSEU149 | Light chain | Anti-It-3 | US20110177087 SEQ ID NO: 23 | 26418 |
| | variable region | | | |
| PSEU150 | Light chain | Anti-It-4 | US20110177087 SEQ ID NO: 24 | 26419 |
| | variable region | | | |
| PSEU151 | Light chain | Anti-It-5 | US20110177087 SEQ ID NO: 25 | 26420 |
| | variable region | | | |
| PSEU152 | Light chain | Anti-It-6 | US20110177087 SEQ ID NO: 26 | 26421 |
| | variable region | | | |
| PSEU153 | Light chain | Anti-170003 | US20110177087 SEQ ID NO: 27 | 26422 |
| | variable region | | | |
| PSEU154 | Light chain | Anti-170006 | US20110177087 SEQ ID NO: 28 | 26423 |
| | variable region | | | |
| PSEU155 | Light chain | Anti-Pa01 | US20110177087 SEQ ID NO: 29 | 26424 |
| | variable region | | | |
| PSEU156 | Light chain | Anti- | US20110177087 SEQ ID NO: 30 | 26425 |
| | variable region | IATS016 | | |
| PSEU157 | Light chain | | US20090191186 SEQ ID NO: 2 | 26426 |
| | variable region | | | |
| PSEU158 | Light chain | | US20090191186 SEQ ID NO: 12 | 26427 |
| | variable region | | | |
| PSEU159 | Light chain | | US20090191186 SEQ ID NO: 8 | 26428 |
| | variable region | | | |
| PSEU160 | Light chain | | US20090191186 SEQ ID NO: 10 | 26429 |
| | variable region | | | |
| PSEU161 | Light chain | | US20090191186 SEQ ID NO: 6 | 26430 |
| | variable region | | | |
| PSEU162 | Light chain | | US20090191186 SEQ ID NO: 37 | 26431 |
| | variable region | | | |
| PSEU163 | Light chain | | US20090191186 SEQ ID NO: 18 | 26432 |
| | variable region | | | |
| PSEU164 | Light chain | | US20090191186 SEQ ID NO: 24 | 26433 |
| | variable region | | | |
| PSEU165 | Light chain | | US20090191186 SEQ ID NO: 20 | 26434 |
| | variable region | | | |
| PSEU166 | Light chain | | US20090191186 SEQ ID NO: 36 | 26435 |
| | variable region | | | |
| PSEU167 | Light chain | | US20090191186 SEQ ID NO: 28 | 26436 |
| | variable region | | | |
| PSEU168 | Light chain | | US20090191186 SEQ ID NO: 30 | 26437 |
| | variable region | | | |
| PSEU169 | Light chain | | US20090191186 SEQ ID NO: 34 | 26438 |
| | variable region | | | |
| PSEU170 | Light chain | | US20090191186 SEQ ID NO: 32 | 26439 |
| | variable region | | | |
| PSEU171 | Light chain | V2L2 | WO2014074528 SEQ ID NO: 217 | 26440 |
| | variable region | | | |
| PSEU172 | Light chain | 2409 | WO2013024905 SEQ ID NO: 15 | 26441 |
| | variable region | | | |
| PSEU173 | Light chain | 2453 | WO2013024905 SEQ ID NO: 23 | 26442 |
| | variable region | | | |
| PSEU174 | Light chain | S20 | U.S. Pat. No. 7,972,845 SEQ ID NO: 4 | 26443 |
| | variable region | | | |
| PSEU175 | Light chain | Fab 13.37 | US20150044215 SEQ ID NO: 143 | 26444 |
| | variable region | | | |
| PSEU176 | Light chain | Fab 26.24 | US20150044215 SEQ ID NO: 145 | 26445 |
| | variable region | | | |
| PSEU177 | Light chain | Fab 35.36 | US20150044215 SEQ ID NO: 145 | 26446 |
| | variable region | | | |
| PSEU178 | Light chain variable | mAbs LST-002 | U.S. Pat. No. 8,653,242 SEQ ID NO: 32 | 26447 |
| | region majority | | | |
| PSEU179 | Light chain variable | mAbs LST-006 | U.S. Pat. No. 8,653,242 SEQ ID NO: 55 | 26448 |
| | region majority | | | |
| PSEU180 | Light chain variable | mAbs LST-002 | U.S. Pat. No. 8,653,242 SEQ ID NO: 51 | 26449 |
| | region minority | | | |
| PSEU181 | Light chain variable | mAbs LST-007 | U.S. Pat. No. 8,653,242 SEQ ID NO: 56 | 26450 |
| | region minority | | | |
| PSEU182 | Light chain, Anti-P. | | Horn, M. P. et al. “Preclinical In | 26451 |
| | Aeuginosa LPS | | Vitro and In Vivo characterization | |
| | serotype IATS-O11, | | of the fully human monoclonal | |
| | | | IgM antibody KBPA101 specific | |
| | | | for Pseudomonas aeruginosa | |
| | | | serotype IATS-O11”, Antimicrob. | |
| | | | Agents Chemother. 54 (6), 2338- | |
| | | | 2344 (2010) | |
| PSEU183 | Light kappa chain | KB0001 | U.S. Pat. No. 8,044,181 SEQ ID NO: 10 | 26452 |
| | variable region | | | |
| PSEU184 | Light kappa chain | KB0001 | U.S. Pat. No. 8,044,181 SEQ ID NO: 2 | 26453 |
| | variable region | | | |
| PSEU185 | Light kappa chain | KB0001 | U.S. Pat. No. 8,044,181 SEQ ID NO: 4 | 26454 |
| | variable region | | | |
| PSEU186 | Light kappa chain | KB0001 | U.S. Pat. No. 8,044,181 SEQ ID NO: 6 | 26455 |
| | variable region | | | |
| PSEU187 | Light kappa chain | KB0001 | U.S. Pat. No. 8,044,181 SEQ ID NO: 8 | 26456 |
| | variable region | | | |
| PSEU188 | Monovalent nanobody | 5H01 | US20150044215 SEQ ID NO: 1 | 26457 |
| PSEU189 | Monovalent nanobody | 7C10 | US20150044215 SEQ ID NO: 2 | 26458 |
| PSEU190 | Monovalent nanobody | 1E11 | US20150044215 SEQ ID NO: 3 | 26459 |
| PSEU191 | Monovalent nanobody | 2B02 | US20150044215 SEQ ID NO: 4 | 26460 |
| PSEU192 | Monovalent nanobody | 2B10 | US20150044215 SEQ ID NO: 5 | 26461 |
| PSEU193 | Monovalent nanobody | 2G09 | US20150044215 SEQ ID NO: 6 | 26462 |
| PSEU194 | Monovalent nanobody | 6B05 | US20150044215 SEQ ID NO: 7 | 26463 |
| PSEU195 | Monovalent nanobody | 10C05 | US20150044215 SEQ ID NO: 8 | 26464 |
| PSEU196 | Monovalent nanobody | 11B09 | US20150044215 SEQ ID NO: 9 | 26465 |
| PSEU197 | Monovalent nanobody | 14E10 | US20150044215 SEQ ID NO: 10 | 26466 |
| PSEU198 | Monovalent nanobody | 7E09 | US20150044215 SEQ ID NO: 11 | 26467 |
| PSEU199 | Monovalent nanobody | 13F07 | US20150044215 SEQ ID NO: 12 | 26468 |
| PSEU200 | Monovalent nanobody | 3B11 | US20150044215 SEQ ID NO: 13 | 26469 |
| PSEU201 | Monovalent nanobody | 4C03 | US20150044215 SEQ ID NO: 14 | 26470 |
| PSEU202 | Monovalent nanobody | 4G10 | US20150044215 SEQ ID NO: 15 | 26471 |
| PSEU203 | Monovalent nanobody | 12B02 | US20150044215 SEQ ID NO: 16 | 26472 |
| PSEU204 | Monovalent nanobody | 14B10 | US20150044215 SEQ ID NO: 17 | 26473 |
| PSEU205 | Monovalent nanobody | 3E10 | US20150044215 SEQ ID NO: 18 | 26474 |
| PSEU206 | Monovalent nanobody | 5E02 | US20150044215 SEQ ID NO: 19 | 26475 |
| PSEU207 | Scfv-Fc | W4-M1 | WO2014074528 SEQ ID NO: 78 | 26476 |
| PSEU208 | Scfv-Fc | W4-M5 | WO2014074528 SEQ ID NO: 79 | 26477 |
| PSEU209 | Scfv-Fc | W4-M6 | WO2014074528 SEQ ID NO: 80 | 26478 |
| PSEU210 | Scfv-Fc | W4-M7 | WO2014074528 SEQ ID NO: 81 | 26479 |
| PSEU211 | Scfv-Fc | W4-M8 | WO2014074528 SEQ ID NO: 82 | 26480 |
| PSEU212 | Scfv-Fc | W4-M9 | WO2014074528 SEQ ID NO: 83 | 26481 |
| PSEU213 | Scfv-Fc | W4-M11 | WO2014074528 SEQ ID NO: 84 | 26482 |
| PSEU214 | Scfv-Fc | W4-M12 | WO2014074528 SEQ ID NO: 85 | 26483 |
| PSEU215 | Scfv-Fc | W4-M14 | WO2014074528 SEQ ID NO: 86 | 26484 |
| PSEU216 | Scfv-Fc | W4-M15 | WO2014074528 SEQ ID NO: 87 | 26485 |
| PSEU217 | Scfv-Fc | W4-M16 | WO2014074528 SEQ ID NO: 88 | 26486 |
| PSEU218 | Scfv-Fc | W4-M17 | WO2014074528 SEQ ID NO: 89 | 26487 |
| PSEU219 | Scfv-Fc | W4-M19 | WO2014074528 SEQ ID NO: 90 | 26488 |
| PSEU220 | Scfv-Fc | W4-M20 | WO2014074528 SEQ ID NO: 91 | 26489 |
| PSEU221 | Sefv-Fc | W4-M4 | WO2014074528 SEQ ID NO: 92 | 26490 |
| PSEU222 | Scfv-Fc | W4-M10 | WO2014074528 SEQ ID NO: 93 | 26491 |
| PSEU223 | Scfv-Fc | W4-HC1-LCP | WO2014074528 SEQ ID NO: 94 | 26492 |
| PSEU224 | Scfv-Fc | W4-HC1-LC7 | WO2014074528 SEQ ID NO: 95 | 26493 |
| PSEU225 | Scfv-Fc | W4-HC2-LC7 | WO2014074528 SEQ ID NO: 96 | 26494 |
| PSEU226 | Scfv-Fc | W4-HC3-LCP | WO2014074528 SEQ ID NO: 97 | 26495 |
| PSEU227 | Scfv-Fc | W4-HC4-LCP | WO2014074528 SEQ ID NO: 98 | 26496 |
| PSEU228 | Scfv-Fc | W4-HC5-LCP | WO2014074528 SEQ ID NO: 99 | 26497 |
| PSEU229 | Scfv-Fc | W4-HC5-LC7 | WO2014074528 SEQ ID NO: 100 | 26498 |
| PSEU230 | Scfv-Fc | W4-HC7-LCP | WO2014074528 SEQ ID NO: 101 | 26499 |
| PSEU231 | Scfv-Fc | W4-VH1-VL8 | WO2014074528 SEQ ID NO: 102 | 26500 |
| PSEU232 | Scfv-Fc | W4-VH2-VLP | WO2014074528 SEQ ID NO: 103 | 26501 |
| PSEU233 | Scfv-Fc | W4-VH2-VL8 | WO2014074528 SEQ ID NO: 104 | 26502 |
| PSEU234 | Scfv-Fc | W4-VH3-VL7 | WO2014074528 SEQ ID NO: 105 | 26503 |
| PSEU235 | Scfv-Fc | W4-VH3-VL8 | WO2014074528 SEQ ID NO: 106 | 26504 |
| PSEU236 | Scfv-Fc | W4-VH5-VL8 | WO2014074528 SEQ ID NO: 107 | 26505 |
| PSEU237 | Scfv-Fc | W4-VH6-VL7 | WO2014074528 SEQ ID NO: 108 | 26506 |
| PSEU238 | Scfv-Fc | W4-VH6-VL8 | WO2014074528 SEQ ID NO: 109 | 26507 |
| PSEU239 | Scfv-Fc | W4-VH6-VLP | WO2014074528 SEQ ID NO: 110 | 26508 |
| PSEU240 | Scfv-Fc | W4-VH7-VLP | WO2014074528 SEQ ID NO: 111 | 26509 |
| PSEU241 | Scfv-Fc | W4-VH7-VL7 | WO2014074528 SEQ ID NO: 112 | 26510 |
| PSEU242 | Scfv-Fc | W4-VH7-VL8 | WO2014074528 SEQ ID NO: 113 | 26511 |
| PSEU243 | Scfv-Fc | W4-VH9-VLP | WO2014074528 SEQ ID NO: 114 | 26512 |
| PSEU244 | Scfv-Fc | W4-VH10-VLP | WO2014074528 SEQ ID NO: 115 | 26513 |
| PSEU245 | Scfv-Fc | W4-VH11-VLP | WO2014074528 SEQ ID NO: 116 | 26514 |
| PSEU246 | Scfv-Fc | W4-VH12-VLP | WO2014074528 SEQ ID NO: 117 | 26515 |
| PSEU247 | Scfv-Fc | W4-VH15-VLP | WO2014074528 SEQ ID NO: 118 | 26516 |
| PSEU248 | Scfv-Fc | W4-VH16-VLP | WO2014074528 SEQ ID NO: 119 | 26517 |
| PSEU249 | Scfv-Fc | W4-VH20-VLP | WO2014074528 SEQ ID NO: 120 | 26518 |
| PSEU250 | Scfv-Fc | W4-VH31-VLP | WO2014074528 SEQ ID NO: 121 | 26519 |
| PSEU251 | Scfv-Fc | W4-VH37-VLP | WO2014074528 SEQ ID NO: 122 | 26520 |
| PSEU252 | Scfv-Fc | W4-VH41-VLP | WO2014074528 SEQ ID NO: 123 | 26521 |
| PSEU253 | Scfv-Fc | W4-VH42-VLP | WO2014074528 SEQ ID NO: 124 | 26522 |
| PSEU254 | Scfv-Fc | W4-VH35-VLP | WO2014074528 SEQ ID NO: 125 | 26523 |
| PSEU255 | Scfv-Fc | W4-VH36-VLP | WO2014074528 SEQ ID NO: 126 | 26524 |
| PSEU256 | Scfv-Fc | W4-VH52-VLP | WO2014074528 SEQ ID NO: 127 | 26525 |
| PSEU257 | Scfv-Fc | W4-VH53-VLP | WO2014074528 SEQ ID NO: 128 | 26526 |
| PSEU258 | Scfv-Fc | W4-VH54-VLP | WO2014074528 SEQ ID NO: 129 | 26527 |
| PSEU259 | Scfv-Fc | W4-VH55-VLP | WO2014074528 SEQ ID NO: 130 | 26528 |
| PSEU260 | Scfv-Fc | W4-VH56-VLP | WO2014074528 SEQ ID NO: 131 | 26529 |
| PSEU261 | Scfv-Fc | W4-VH57-VLP | WO2014074528 SEQ ID NO: 132 | 26530 |
| PSEU262 | Scfv-Fc | W4-VH58-VLP | WO2014074528 SEQ ID NO: 133 | 26531 |
| PSEU263 | Scfv-Fc | W4-VH60-VLP | WO2014074528 SEQ ID NO: 134 | 26532 |
| PSEU264 | Scfv-Fc | W4-VH61-VLP | WO2014074528 SEQ ID NO: 135 | 26533 |
| PSEU265 | Scfv-Fc | W4-VH62-VLP | WO2014074528 SEQ ID NO: 136 | 26534 |
| PSEU266 | Scfv-Fc | W4-VH63-VLP | WO2014074528 SEQ ID NO: 137 | 26535 |
| PSEU267 | Scfv-Fc | W4-VH64-VLP | WO2014074528 SEQ ID NO: 138 | 26536 |
| PSEU268 | Scfv-Fc | W4-VH65-VLP | WO2014074528 SEQ ID NO: 139 | 26537 |
| PSEU269 | Scfv-Fc | W4-VH66-VLP | WO2014074528 SEQ ID NO: 140 | 26538 |
| PSEU270 | Scfv-Fc | W4-VH67-VLP | WO2014074528 SEQ ID NO: 141 | 26539 |
| PSEU271 | Scfv-Fc | W4-VH69-VLP | WO2014074528 SEQ ID NO: 142 | 26540 |
| PSEU272 | Scfv-Fc | W4-VH70-VLP | WO2014074528 SEQ ID NO: 143 | 26541 |
| PSEU273 | Scfv-Fc | W4-VH72-VLP | WO2014074528 SEQ ID NO: 144 | 26542 |
| PSEU274 | Scfv-Fc | W4-VH79-VLP | WO2014074528 SEQ ID NO: 145 | 26543 |
| PSEU275 | Scfv-Fc | W4-VH80-VLP | WO2014074528 SEQ ID NO: 146 | 26544 |
| PSEU276 | Scfv-Fc | W4-M9 | WO2014074528 SEQ ID NO: 152 | 26545 |
| PSEU277 | Scfv-Fc | Psl0170 | WO2014074528 SEQ ID NO: 245 | 26546 |
| PSEU278 | Scfv-Fc | Psl0304 | WO2014074528 SEQ ID NO: 246 | 26547 |
| PSEU279 | Scfv-Fc | Psl0348 | WO2014074528 SEQ ID NO: 247 | 26548 |
| PSEU280 | Scfv-Fc | Psl0573 | WO2014074528 SEQ ID NO: 248 | 26549 |
| PSEU281 | Scfv-Fc | Psl0574 | WO2014074528 SEQ ID NO: 249 | 26550 |
| PSEU282 | Scfv-Fc | Psl0582 | WO2014074528 SEQ ID NO: 250 | 26551 |
| PSEU283 | Scfv-Fc | Psl0584 | WO2014074528 SEQ ID NO: 251 | 26552 |
| PSEU284 | Scfv-Fc | Psl0585 | WO2014074528 SEQ ID NO: 252 | 26553 |
| PSEU285 | Scfv-Fc | Psl0589 | WO2014074528 SEQ ID NO: 253 | 26554 |

In some embodiments, the payload region of the viral particle comprises one or more nucleic acid sequences encoding one or more of the payload antibody polypeptides listed in Table 44 against Streptococcus bacteria (STRP1-STRP40; SEQ ID NO: 26555-26594),

TABLE 44
Antibodies against Streptococcus bacteria

| Antibody | | Antibody | Reference | SEQ ID |
|---|---|---|---|---|
| No. | Description | Name | Information | NO |
| STRP1 | Heavy chain variable region, | | U.S. Pat. No. 7,625,561 | 26555 |
| | Diabody for Streptococcus | | SEQ ID NO: 5 | |
| STRP2 | Heavy chain variable region, | | U.S. Pat. No. 7,625,561 | 26556 |
| | Diabody for Streptococcus | | SEQ ID NO: 3 | |
| STRP3 | Heavy chain variable region, | | U.S. Pat. No. 7,625,561 | 26557 |
| | Diabody for Streptococcus | | SEQ ID NO: 7 | |
| STRP4 | Heavy chain variable region, | DP-54 | Lucas, A. H. “Combinatorial library | 26558 |
| | partial, Streptococcus | | cloning of human antibodies to | |
| | pneumoniae | | Streptococcus pneumoniae capsular | |
| | | | polysaccharides: variable region | |
| | | | primary structures and evidence for | |
| | | | somatic mutation of Fab fragments | |
| | | | specific for capsular serotypes 6B, | |
| | | | 14, and 23F” Infect. Immun. 69 (2), | |
| | | | 853-864 (2001), NCBI Accession # | |
| | | | AAD48823 | |
| STRP5 | Heavy chain variable | DP-35 | Lucas, A. H. “Combinatorial library | 26559 |
| | region, partial, | | cloning of human antibodies to | |
| | Streptococcus pneumoniae | | Streptococcus pneumoniae capsular | |
| | | | polysaccharides: variable region | |
| | | | primary structures and evidence for | |
| | | | somatic mutation of Fab fragments | |
| | | | specific for capsular serotypes 6B, | |
| | | | 14, and 23F,” Infect. Immun. 69 (2), | |
| | | | 853-864 (2001), NCBI Accession # | |
| | | | AAD48825 | |
| STRP6 | Heavy chain variable | DP-47 | Lucas, A. H. “Combinatorial library | 26560 |
| | region, partial, | | cloning of human antibodies to | |
| | Streptococcus pneumoniae | | Streptococcus pneumoniae capsular | |
| | | | polysaccharides: variable region | |
| | | | primary structures and evidence for | |
| | | | somatic mutation of Fab fragments | |
| | | | specific for capsular serotypes 6B, | |
| | | | 14, and 23F” Infect. Immun. 69 (2), | |
| | | | 853-864 (2001), NCBI Accession # | |
| | | | AAD48827 | |
| STRP7 | Heavy chain variable | DP-47 | Lucas, A. H. “Combinatorial library | 26561 |
| | region, partial, | | cloning of human antibodies to | |
| | Streptococcus pneumoniae | | Streptococcus pneumoniae capsular | |
| | | | polysaccharides: variable region | |
| | | | primary structures and evidence for | |
| | | | somatic mutation of Fab fragments | |
| | | | specific for capsular serotypes 6B, | |
| | | | 14, and 23F” Infect. Immun. 69 (2), | |
| | | | 853-864 (2001), NCBI Accession # | |
| | | | AAD48828 | |
| STRP8 | Heavy chain variable | LSG-6.1 | Lucas, A. H. “Combinatorial library | 26562 |
| | region, partial, | | cloning of human antibodies to | |
| | Streptococcus pneumoniae | | Streptococcus pneumoniae capsular | |
| | | | polysaccharides: variable region | |
| | | | primary structures and evidence for | |
| | | | somatic mutation of Fab fragments | |
| | | | specific for capsular serotypes 6B, | |
| | | | 14, and 23F” Infect. Immun. 69 (2), | |
| | | | 853-864 (2001), NCBI Accession # | |
| | | | AAD48830 | |
| STRP9 | Heavy chain variable | LSG6.1 | Lucas, A. H. “Combinatorial library | 26563 |
| | region, partial, | | cloning of human antibodies to | |
| | Streptococcus pneumoniae | | Streptococcus pneumoniae capsular | |
| | | | polysaccharides: variable region | |
| | | | primary structures and evidence for | |
| | | | somatic mutation of Fab fragments | |
| | | | specific for capsular serotypes 6B, | |
| | | | 14, and 23F” Infect. Immun. 69 (2), | |
| | | | 853-864 (2001), NCBI Accession # | |
| | | | AAD48832 | |
| STRP10 | Heavy chain variable | DP-47 | Lucas, A. H. “Combinatorial library | 26564 |
| | region, partial, | | cloning of human antibodies to | |
| | Streptococcus pneumoniae | | Streptococcus pneumoniae capsular | |
| | | | polysaccharides: variable region | |
| | | | primary structures and evidence for | |
| | | | somatic mutation of Fab fragments | |
| | | | specific for capsular serotypes 6B, | |
| | | | 14, and 23F” Infect. Immun. 69 (2), | |
| | | | 853-864 (2001), NCBI Accession # | |
| | | | AAD48835 | |
| STRP11 | Heavy chain variable region, | humanized | U.S. Pat. No. 7,429,381 | 26565 |
| | Streptococcus agalactiae, | Mu-9 | SEQ ID NO: 14 | |
| | Legionella pneumophilia, | | | |
| | Streptococcus pyogenes, | | | |
| | Escherichia coli, | | | |
| | Neisseria gonorrhoeae, | | | |
| | Neisseria meningitidis, | | | |
| | Pneumococcus, | | | |
| | Hemophilis influenzae B, | | | |
| | Treponema pallidum, | | | |
| | Lyme disease spirochetes, | | | |
| | Pseudomonas aeruginosa, | | | |
| | Mycobacterium leprae, | | | |
| | Brucella abortus and | | | |
| | Mycobacterium tuberculosis. | | | |
| STRP12 | Heavy chain variable region, | Anti-PsaA | US20070003561 | 26566 |
| | Streptococcus pneumoniae | 7-1G9 | SEQ ID NO: 16 | |
| STRP13 | Heavy chain variable region, | Anti-PsaA | US20070003561 | 26567 |
| | Streptococcus pneumoniae | 1-15E5 | SEQ ID NO: 32 | |
| STRP14 | Heavy chain variable region, | Anti-PsaA | US20070003561 | 26568 |
| | Streptococcus pneumoniae | 9A7 | SEQ ID NO: 48 | |
| STRP15 | Heavy chain variable region, | 23f Fab | Bryson, S., “Multitasking | 26569 |
| | Streptococcus pneumoniae | 023.102, | Immunoglobulin V-Genes And | |
| | | chain B | Somatic Div Cdr3 Loops Generate | |
| | | | Binding Sites For Chemically Di | |
| | | | Antigens From Bacterial And Viral | |
| | | | Pathogens” Unpublished”, NCBI | |
| | | | Accession # 4HIE B | |
| STRP16 | Heavy chain variable region, | 5.12.14 | U.S. Pat. No. 5,686,070 | 26570 |
| | Streptococcus pneumoniae, | | SEQ ID NO: 22 | |
| | Escherichia coli, or | | | |
| | Pseudomonas aeruginosa | | | |
| SIRP17 | Heavy chain variable region, | 6G4.2.5 | U.S. Pat. No. 5,686,070 | 26571 |
| | Streptococcus pneumoniae, | | SEQ ID NO: 50 | |
| | Escherichia coli, or | | | |
| | Pseudomonas aeruginosa | | | |
| STRP18 | Heavy chain variable region, | chimeric | U.S. Pat. No. 5,686,070 | 26572 |
| | Streptococcus pneumoniae, | 6G4.2.5 | SEQ ID NO: 58 | |
| | Escherichia coli, or | | | |
| | Pseudomonas aeruginosa | | | |
| STRP19 | Heavy chain, | Mab679 | U.S. Pat. No. 7,429,381 | 26573 |
| | Streptococcus agalactiae, | | SEQ ID NO: 4 | |
| | Legionella pneumophilia, | | | |
| | Streptococcus pyogenes, | | | |
| | Escherichia coli, | | | |
| | Neisseria gonorrhoeae, | | | |
| | Neisseria meningitidis, | | | |
| | Pneumococcus, | | | |
| | Hemophilis influenzae B, | | | |
| | Treponema pallidum, | | | |
| | Lyme disease spirochetes, | | | |
| | Pseudomonas aeruginosa, | | | |
| | Mycobacterium leprae, | | | |
| | Brucella abortus and | | | |
| | Mycobacterium tuberculosis. | | | |
| STRP20 | Light chain variable region, | | U.S. Pat. No. 7,625,561 | 26574 |
| | Diabody for Streptococcus | | SEQ ID NO: 6 | |
| STRP21 | Light chain variable region, | | U.S. Pat. No. 7,625,561 | 26575 |
| | Diabody for Streptococcus | | SEQ ID NO: 8 | |
| STRP22 | Light chain variable region, | | U.S. Pat. No. 7,625,561 | 26576 |
| | Diabody for Streptococcus | | SEQ ID NO: 4 | |
| STRP23 | Light chain variable | A2 | Lucas, A. H. “Combinatorial library | 26577 |
| | region, partial, | | cloning of human antibodies to | |
| | Streptococcus pneumoniae | | Streptococcus pneumoniae capsular | |
| | | | polysaccharides: variable region | |
| | | | primary structures and evidence for | |
| | | | somatic mutation of Fab fragments | |
| | | | specific for capsular serotypes 6B, | |
| | | | 14, and 23F” Infect. Immun. 69 (2), | |
| | | | 853-864 (2001), NCBI Accession # | |
| | | | AAD48824 | |
| STRP24 | Light chain variable | B3 | Lucas, A. H. “Combinatorial library | 26578 |
| | region, partial, | | cloning of human antibodies to | |
| | Streptococcus pneumoniae | | Streptococcus pneumoniae capsular | |
| | | | polysaccharides: variable region | |
| | | | primary structures and evidence for | |
| | | | somatic mutation of Fab fragments | |
| | | | specific for capsular serotypes 6B, | |
| | | | 14, and 23F” Infect. Immun. 69 (2), | |
| | | | 853-864 (2001), NCBI Accession # | |
| | | | AAD48822 | |
| STRP25 | Light chain variable | A23 | Lucas, A. H. “Combinatorial library | 26579 |
| | region, partial, | | cloning of human antibodies to | |
| | Streptococcus pneumoniae | | Streptococcus pneumoniae capsular | |
| | | | polysaccharides: variable region | |
| | | | primary structures and evidence for | |
| | | | somatic mutation of Fab fragments | |
| | | | specific for capsular serotypes 6B, | |
| | | | 14, and 23F” Infect. Immun. 69 (2), | |
| | | | 853-864 (2001), NCBI Accession # | |
| | | | AAD48826 | |
| STRP26 | Light chain variable | L2 | Lucas, A. H. “Combinatorial library | 26580 |
| | region, partial, | | cloning of human antibodies to | |
| | Streptococcus pneumoniae | | Streptococcus pneumoniae capsular | |
| | | | polysaccharides: variable region | |
| | | | primary structures and evidence for | |
| | | | somatic mutation of Fab fragments | |
| | | | specific for capsular serotypes 6B, | |
| | | | 14, and 23F” Infect. Immun. 69 (2), | |
| | | | 853-864 (2001), NCBI Accession # | |
| | | | AAD48829 | |
| STRP27 | Light chain variable | DPL5 | Lucas, A. H. “Combinatorial library | 26581 |
| | region, partial, | | cloning of human antibodies to | |
| | Streptococcus pneumoniae | | Streptococcus pneumoniae capsular | |
| | | | polysaccharides: variable region | |
| | | | primary structures and evidence for | |
| | | | somatic mutation of Fab fragments | |
| | | | specific for capsular serotypes 6B, | |
| | | | 14, and 23F” Infect. Immun. 69 (2), | |
| | | | 853-864 (2001), NCBI Accession # | |
| | | | AAD48831 | |
| STRP28 | Light chain variable | DPL5 | Lucas, A. H. “Combinatorial library | 26582 |
| | region, partial, | | cloning of human antibodies to | |
| | Streptococcus pneumoniae | | Streptococcus pneumoniae capsular | |
| | | | polysaccharides: variable region | |
| | | | primary structures and evidence for | |
| | | | somatic mutation of Fab fragments | |
| | | | specific for capsular serotypes 6B, | |
| | | | 14, and 23F” Infect. Immun. 69 (2), | |
| | | | 853-864 (2001), NCBI Accession # | |
| | | | AAD48833 | |
| STRP29 | Light chain variable | L2 | Lucas, A. H. “Combinatorial library | 26583 |
| | region, partial, | | cloning of human antibodies to | |
| | Streptococcus pneumoniae | | Streptococcus pneumoniae capsular | |
| | | | polysaccharides: variable region | |
| | | | primary structures and evidence for | |
| | | | somatic mutation of Fab fragments | |
| | | | specific for capsular serotypes 6B, | |
| | | | 14, and 23F” Infect. Immun. 69 (2), | |
| | | | 853-864 (2001), NCBI Accession # | |
| | | | AAD48834 | |
| STRP30 | Light chain variable | Anti-PsaA | US20070003561 | 26584 |
| | region, partial, | 7-1G9 | SEQ ID NO: 8 | |
| | Streptococcus pneumoniae | | | |
| STRP31 | Light chain variable | Anti-PsaA | US20070003561 | 26585 |
| | region, partial, | 1-15E5 | SEQ ID NO: 24 | |
| | Streptococcus pneumoniae | | | |
| STRP32 | Light chain variable | Anti-pSaA | US20070003561 | 26586 |
| | region, partial, | 9A7 | SEQ ID NO: 40 | |
| | Streptococcus pneumoniae | | | |
| STRP33 | Light chain variable region, | 5.12.14 | U.S. Pat. No. 5,686,070 | 26587 |
| | Streptococcus pneumoniae, | | SEQ ID NO: 20 | |
| | Escherichia coli, or | | | |
| | Pseudomonas aeruginosa | | | |
| STRP34 | Light chain variable region, | 6G4.2.5 | U.S. Pat. No. 5,686,070 | 26588 |
| | Streptococcus pneumoniae, | | SEQ ID NO: 48 | |
| | Escherichia coli, or | | | |
| | Pseudomonas aeruginosa | | | |
| STRP35 | Light chain variable region, | chimeric | U.S. Pat. No. 5,686,070 | 26589 |
| | Streptococcus pneumoniae, | 6G4.2.5 | SEQ ID NO: 56 | |
| | Escherichia coli, or | | | |
| | Pseudomonas aeruginosa | | | |
| STRP36 | Light chain, | Mab679 | U.S. Pat. No. 7,429,381 | 26590 |
| | Streptococcus agalactiae, | | SEQ ID NO: 2 | |
| | Legionella pneumophilia, | | | |
| | Streptococcus pyogenes, | | | |
| | Escherichia coli, | | | |
| | Neisseria gonorrhoeae, | | | |
| | Neisseria meningitidis, | | | |
| | Pneumococcus, | | | |
| | Hemophilis influenzae B, | | | |
| | Treponema pallidum, | | | |
| | Lyme disease spirochetes, | | | |
| | Pseudomonas aeruginosa, | | | |
| | Mycobacterium leprae, | | | |
| | Brucella abortus and | | | |
| | Mycobacterium tuberculosis | | | |
| STRP37 | scFv, Streptococcus agalactiae, | Mab679 | U.S. Pat. No. 7,429,381 | 26591 |
| | Legionella pneumophilia, | | SEQ ID NO: 6 | |
| | Streptococcus pyogenes, | | | |
| | Escherichia coli, | | | |
| | Neisseria gonorrhoeae, | | | |
| | Neisseria meningitidis, | | | |
| | Pneumococcus, | | | |
| | Hemophilis influenzae B, | | | |
| | Treponema pallidum, | | | |
| | Lyme disease spirochetes, | | | |
| | Pseudomonas aeruginosa, | | | |
| | Mycobacterium leprae, | | | |
| | Brucella abortus and | | | |
| | Mycobacterium tuberculosis | | | |
| STRP38 | scFv, Streptococcus agalactiae, | Mu-9V | U.S. Pat. No. 7,429,381 | 26592 |
| | Legionella pneumophilia, | | SEQ ID NO: 8 | |
| | Streptococcus pyogenes, | | | |
| | Escherichia coli, | | | |
| | Neisseria gonorrhoeae, | | | |
| | Neisseria meningitidis, | | | |
| | Pneumococcus, | | | |
| | Hemophilis influenzae B, | | | |
| | Treponema pallidum, | | | |
| | Lyme disease spirochetes, | | | |
| | Pseudomonas aeruginosa, | | | |
| | Mycobacterium leprae, | | | |
| | Brucella abortus and | | | |
| | Mycobacterium tuberculosis | | | |
| STRP39 | scFv, Streptococcus agalactiae, | Mu-9V | U.S. Pat. No. 7,429,381 | 26593 |
| | Legionella pneumophilia, | | SEQ ID NO: 10 | |
| | Streptococcus pyogenes, | | | |
| | Escherichia coli, | | | |
| | Neisseria gonorrhoeae, | | | |
| | Neisseria meningitidis, | | | |
| | Pneumococcus, | | | |
| | Hemophilis influenzae B, | | | |
| | Treponema pallidum, | | | |
| | Lyme disease spirochetes, | | | |
| | Pseudomonas aeruginosa, | | | |
| | Mycobacterium leprae, | | | |
| | Brucella abortus and | | | |
| | Mycobacterium tuberculosis | | | |
| STRP40 | scFv, Streptococcus agalactiae, | humanized | U.S. Pat. No. 7,429,381 | 26594 |
| | Legionella pneumophilia, | Mu-9 | SEQ ID NO: 12 | |
| | Streptococcus pyogenes, | | | |
| | Escherichia coli, | | | |
| | Neisseria gonorrhoeae, | | | |
| | Neisseria meningitidis, | | | |
| | Pneumococcus, | | | |
| | Hemophilis influenzae B, | | | |
| | Treponema pallidum, | | | |
| | Lyme disease spirochetes, | | | |
| | Pseudomonas aeruginosa, | | | |
| | Mycobacterium leprae, | | | |
| | Brucella abortus and | | | |
| | Mycobacterium tuberculosis | | | |

In some embodiments, the payload region of the viral particle comprises one or more nucleic acid sequences, fragments or variants thereof or encodes one or more polypeptides, fragments or variants thereof described in US Pub No. US20040198960 and US20130195876, the contents of each of which are herein incorporated by reference in their entirety, against Streptococcus Pneumoniae infection.

In some embodiments, the payload region of the viral particle comprises one or more nucleic acid sequences, fragments or variants encoding Afelimomab, fragments or variants thereof for treating a disease and/or disorder or preventing a disease and/or disorder. As a non-limiting example, the disease and/or disorder is sepsis.

In some embodiments, the payload region of the viral particle comprises one or more nucleic acid sequences, fragments or variants encoding Nebacumab, fragments or variants thereof for treating a disease and/or disorder or preventing a disease and/or disorder. As a non-limiting example, the disease and/or disorder is sepsis.

In some embodiments, the payload region of the viral particle comprises one or more nucleic acid sequences encoding one or more of the payload antibody polypeptides listed in Table 45 against Staphylococcal bacteria and related bacteria (STPH1-STPH249; SEQ ID NO: 26595-26843).

TABLE 45
Antibodies against Staphylococcal bacteria and related bacteria

| Antibody | | Antibody | Reference | SEQ ID |
|---|---|---|---|---|
| No. | Description | Name | Information | NO |
| STPH1 | Heavy chain variable region, S. aureus | | U.S. Pat. No. 8,609,102 | 26595 |
| | | | SEQ ID NO: 2 | |
| STPH2 | Heavy chain variable region, S. aureus | | U.S. Pat. No. 8,609,102 | 26596 |
| | | | SEQ ID NO: 6 | |
| STPH3 | Heavy chain variable region, S. aureus | SAR279356 | U.S. Pat. No. 7,786,255 | 26597 |
| | or S. epidermidis, E. coli, Yersinia | | SEQ ID NO: 1 | |
| | pestis (Y. pestis), Y. entercolitica, | | | |
| | Xanthomnonas axonopodis (X. | | | |
| | axonopodis), Pseudonmonas | | | |
| | fuorescerns (P. | | | |
| | fluorescens), Actinobacillus | | | |
| | actinomycetemcomitans (A. | | | |
| | actinomycetemcomitans), A. | | | |
| | pleuropneumoniae, Ralstonia | | | |
| | solanacearum (R. | | | |
| | solanacearum), Bordetella pertussis (B. | | | |
| | pertussis), B. parapertussis or B. | | | |
| | bronchiseptica | | | |
| STPH4 | Heavy chain variable region, S. aureus | SAR279356 | US20110002932 SEQ ID | 26598 |
| | or S. epidermidis, E. coli, Yersinia | | NO: 1 | |
| | pestis (Y. pestis), Y. entercolitica, | | | |
| | Xanthomnonas axonopodis (X. | | | |
| | axonopodis), Pseudonmonas | | | |
| | fuorescerns (P. | | | |
| | fluorescens), Actinobacillus | | | |
| | actinomycetemcomitans (A. | | | |
| | actinomycetemcomitans), A. | | | |
| | pleuropneumoniae, Ralstonia | | | |
| | solanacearum (R. | | | |
| | solanacearum), Bordetella pertussis (B. | | | |
| | pertussis), B. parapertussis or B. | | | |
| | bronchiseptica | | | |
| STPH5 | Heavy chain variable region, S. | 108-1 | U.S. Pat. No. 8,475,798 | 26599 |
| | epidermidis | | SEQ ID NO: 18 | |
| STPH6 | Heavy chain variable region, S. | 108-36 | U.S. Pat. No. 8,475,798 | 26600 |
| | epidermidis | | SEQ ID NO: 22 | |
| STPH7 | Heavy chain variable region, S. | 110-15 | U.S. Pat. No. 8,475,798 | 26601 |
| | epidermidis | | SEQ ID NO: 26 | |
| STPH8 | Heavy chain variable region, S. | 108-1VH- | U.S. Pat. No. 8,475,798 | 26602 |
| | epidermidis | Hu | SEQ ID NO: 28 | |
| STPH9 | Heavy chain variable region, S. | 108-36VH- | U.S. Pat. No. 8,475,798 | 26603 |
| | epidermidis | Hu | SEQ ID NO: 30 | |
| STPH10 | Heavy chain variable region, S. | 110-15VH- | U.S. Pat. No. 8,475,798 | 26604 |
| | epidermidis | Hu | SEQ ID NO: 32 | |
| STPH11 | Heavy chain variable region, | Pagibaximab | U.S. Pat. No. 8,372,958 | 26605 |
| | Staphylococcal sepsis | | SEQ ID NO: 87 | |
| STPH12 | Heavy chain variable region, | Pagibaximab | U.S. Pat. No. 8,372,958 | 26606 |
| | Staphylococcal sepsis | | SEQ ID NO: 12 | |
| STPH13 | Heavy chain variable region, | Pagibaximab | U.S. Pat. No. 8,372,958 | 26607 |
| | Staphylococcal sepsis | | SEQ ID NO: 17 | |
| STPH14 | Heavy chain variable region, | F628 | U.S. Pat. No. 8,912,314 | 26608 |
| | Staphylococci such as S. aureus and S. | | SEQ ID NO: 3 | |
| | epidermidis, E. coli such as E. | | | |
| | coli strains O157:H7 and | | | |
| | CFT073, Yersinia pestis, Yersinia | | | |
| | entercolitica, Xanthomonas axonopodis, | | | |
| | Pseudomonas fluorescens (all of which | | | |
| | are sequenced species with complete | | | |
| | pgaABCD loci), and Actinobacillus | | | |
| | actinomycetemcomitans (AA), | | | |
| | Actinobacillus pleuropneumoniae (Ap), | | | |
| | Ralstonia solanacearum (e.g., megaplasmid | | | |
| | form), Bordetella pertussis, Bordetella | | | |
| | parapertussis and Bordetella | | | |
| | bronchiseptica | | | |
| STPH15 | Heavy chain variable region, | F630 | U.S. Pat. No. 8,912,314 | 26609 |
| | Staphylococci such as S. aureus and S. | | SEQ ID NO: 5 | |
| | epidermidis, E. coli such as E. | | | |
| | coli strains O157:H7 and | | | |
| | CFT073, Yersinia pestis, Yersinia | | | |
| | entercolitica, Xanthomonas axonopodis, | | | |
| | Pseudomonas fluorescens (all of which | | | |
| | are sequenced species with complete | | | |
| | pgaABCD loci), and Actinobacillus | | | |
| | actinomycetemcomitans (AA), | | | |
| | Actinobacillus pleuropneumoniae (Ap), | | | |
| | Ralstonia solanacearum (e.g., megaplasmid | | | |
| | form), Bordetella pertussis, Bordetella | | | |
| | parapertussis and Bordetella | | | |
| | bronchiseptica | | | |
| STPH16 | Heavy chain variable region, | F598 | U.S. Pat. No. 8,912,314 | 26610 |
| | Staphylococci such as S. aureus and S. | | SEQ ID NO: 55 | |
| | epidermidis, E. coli such as E. | | | |
| | coli strains O157:H7 and | | | |
| | CFT073, Yersinia pestis, Yersinia | | | |
| | entercolitica, Xanthomonas axonopodis, | | | |
| | Pseudomonas fluorescens (all of which | | | |
| | are sequenced species with complete | | | |
| | pgaABCD loci), and Actinobacillus | | | |
| | actinomycetemcomitans (AA), | | | |
| | Actinobacillus pleuropneumoniae (Ap), | | | |
| | Ralstonia solanacearum (e.g., megaplasmid | | | |
| | form), Bordetella pertussis, Bordetella | | | |
| | parapertussis and Bordetella | | | |
| | bronchiseptica | | | |
| STPH17 | Heavy chain variable region, | F628 | U.S. Pat. No. 8,912,314 | 26611 |
| | Staphylococci such as S. aureus and S. | | SEQ ID NO: 58 | |
| | epidermidis, E. coli such as E. | | | |
| | coli strains O157:H7 and | | | |
| | CFT073, Yersinia pestis, Yersinia | | | |
| | entercolitica, Xanthomonas axonopodis, | | | |
| | Pseudomonas fluorescens (all of which | | | |
| | are sequenced species with complete | | | |
| | pgaABCD loci), and Actinobacillus | | | |
| | actinomycetemcomitans (AA), | | | |
| | Actinobacillus pleuropneumoniae (Ap), | | | |
| | Ralstonia solanacearum (e.g., megaplasmid | | | |
| | form), Bordetella pertussis, Bordetella | | | |
| | parapertussis and Bordetella | | | |
| | bronchiseptica | | | |
| STPH18 | Heavy chain variable region, | F598 | U.S. Pat. No. 8,912,314 | 26612 |
| | Staphylococci such as S. aureus and S. | | SEQ ID NO: 1 | |
| | epidermidis, E. coli such as E. | | | |
| | coli strains O157:H7 and | | | |
| | CFT073, Yersinia pestis, Yersinia | | | |
| | entercolitica, Xanthomonas axonopodis, | | | |
| | Pseudomonas fluorescens (all of which | | | |
| | are sequenced species with complete | | | |
| | pgaABCD loci), and Actinobacillus | | | |
| | actinomycetemcomitans (AA), | | | |
| | Actinobacillus pleuropneumoniae (Ap), | | | |
| | Ralstonia solanacearum (e.g., megaplasmid | | | |
| | form), Bordetella pertussis, Bordetella | | | |
| | parapertussis and Bordetella | | | |
| | bronchiseptica | | | |
| STPH19 | Heavy chain variable region, | 108-3BVH- | U.S. Pat. No. 8,475,798 | 26613 |
| | Staphylococcus epidermidis | Hu | SEQ ID NO: 34 | |
| STPH20 | Heavy chain, MRSA, MSSA | 2B2 | U.S. Pat. No. 8,735,554 | 26614 |
| | | | SEQ ID NO: 3 | |
| STPH21 | Heavy chain, MRSA, MSSA | 2G7 | U.S. Pat. No. 8,735,554 | 26615 |
| | | | SEQ ID NO: 5 | |
| STPH22 | Heavy chain, MRSA, MSSA | 3B12 | U.S. Pat. No. 8,735,554 | 26616 |
| | | | SEQ ID NO: 7 | |
| STPH23 | Heavy chain, S. aureus | DF1.1 | U.S. Pat. No. 8,715,673 | 26617 |
| | | | SEQ ID NO: 2 | |
| STPH24 | Heavy chain, S. aureus | DF1 | U.S. Pat. No. 8,715,673 | 26618 |
| | | | SEQ ID NO: 4 | |
| STPH25 | Heavy chain, S. aureus | DF2 | U.S. Pat. No. 8,715,673 | 26619 |
| | | | SEQ ID NO: 35 | |
| STPH26 | Heavy chain, S. aureus | DF3 | U.S. Pat. No. 8,715,673 | 26620 |
| | | | SEQ ID NO: 36 | |
| STPH27 | Heavy chain, S. aureus | DF4 | U.S. Pat. No. 8,715,673 | 26621 |
| | | | SEQ ID NO: 37 | |
| STPH28 | Heavy chain, S. aureus | DF5 | U.S. Pat. No. 8,715,673 | 26622 |
| | | | SEQ ID NO: 38 | |
| STPH29 | Heavy chain, S. aureus | DF6 | U.S. Pat. No. 8,715,673 | 26623 |
| | | | SEQ ID NO: 39 | |
| STPH30 | Heavy chain, S. aureus | DF7 | U.S. Pat. No. 8,715,673 | 26624 |
| | | | SEQ ID NO: 40 | |
| STPH31 | Heavy chain, S. aureus | DF8 | U.S. Pat. No. 8,715,673 | 26625 |
| | | | SEQ ID NO: 41 | |
| STPH32 | Heavy chain, S. aureus | DF9 | U.S. Pat. No. 8,715,673 | 26626 |
| | | | SEQ ID NO: 42 | |
| STPH33 | Heavy chain, S. aureus | DF10 | U.S. Pat. No. 8,715,673 | 26627 |
| | | | SEQ ID NO: 43 | |
| STPH34 | Heavy chain, S. aureus | DF11 | U.S. Pat. No. 8,715,673 | 26628 |
| | | | SEQ ID NO: 44 | |
| STPH35 | Heavy chain, S. aureus | DF12 | U.S. Pat. No. 8,715,673 | 26629 |
| | | | SEQ ID NO: 45 | |
| STPH36 | Heavy chain, S. aureus | DF13 | U.S. Pat. No. 8,715,673 | 26630 |
| | | | SEQ ID NO: 46 | |
| STPH37 | Heavy chain, S. aureus | DF14 | U.S. Pat. No. 8,715,673 | 26631 |
| | | | SEQ ID NO: 47 | |
| STPH38 | Heavy chain, S. aureus | DF15 | U.S. Pat. No. 8,715,673 | 26632 |
| | | | SEQ ID NO: 48 | |
| STPH39 | Heavy chain, S. aureus | DF16 | U.S. Pat. No. 8,715,673 | 26633 |
| | | | SEQ ID NO: 49 | |
| STPH40 | Heavy chain, S. aureus | DF17 | U.S. Pat. No. 8,715,673 | 26634 |
| | | | SEQ ID NO: 50 | |
| STPH41 | Heavy chain, S. aureus | DF18 | U.S. Pat. No. 8,715,673 | 26635 |
| | | | SEQ ID NO: 51 | |
| STPH42 | Heavy chain, S. aureus | DF19 | U.S. Pat. No. 8,715,673 | 26636 |
| | | | SEQ ID NO: 52 | |
| STPH43 | Heavy chain, S. aureus | DF20 | U.S. Pat. No. 8,715,673 | 26637 |
| | | | SEQ ID NO: 53 | |
| STPH44 | Heavy chain, S. aureus and S. | CR2430 | U.S. Pat. No. 8,460,666 | 26638 |
| | epidermidis | | SEQ ID NO: 26 | |
| STPH45 | Heavy chain, S. aureus and S. | CR5132 | U.S. Pat. No. 8,460,666 | 26639 |
| | epidermidis | | SEQ ID NO: 28 | |
| STPH46 | Heavy chain, S. aureus and S. | CR5133 | U.S. Pat. No. 8,460,666 | 26640 |
| | epidermidis | | SEQ ID NO: 30 | |
| STPH47 | Heavy chain, S. aureus and S. | CR6166 | U.S. Pat. No. 8,460,666 | 26641 |
| | epidermidis | | SEQ ID NO: 117 | |
| STPH48 | Heavy chain, S. aureus and S. | CR6171 | U.S. Pat. No. 8,460,666 | 26642 |
| | epidermidis | | SEQ ID NO: 119 | |
| STPH49 | Heavy chain, S. aureus and S. | CR6176 | U.S. Pat. No. 8,460,666 | 26643 |
| | epidermidis | | SEQ ID NO: 121 | |
| STPH50 | Heavy chain, S. aureus and S. | CR6187 | U.S. Pat. No. 8,460,666 | 26644 |
| | epidermidis | | SEQ ID NO: 123 | |
| STPH51 | Heavy chain, S. aureus and S. | CR6193 | U.S. Pat. No. 8,460,666 | 26645 |
| | epidermidis | | SEQ ID NO: 125 | |
| STPH52 | Heavy chain, S. aureus and S. | CR6249 | U.S. Pat. No. 8,460,666 | 26646 |
| | epidermidis | | SEQ ID NO: 127 | |
| STPH53 | Heavy chain, S. aureus and S. | CR6273 | U.S. Pat. No. 8,460,666 | 26647 |
| | epidermidis | | SEQ ID NO: 129 | |
| STPH54 | Heavy chain, S. aureus and S. | CR6389 | U.S. Pat. No. 8,460,666 | 26648 |
| | epidermidis | | SEQ ID NO: 131 | |
| STPH55 | Heavy chain, S. aureus and S. | CR6403 | U.S. Pat. No. 8,460,666 | 26649 |
| | epidermidis | | SEQ ID NO: 133 | |
| STPH56 | Heavy chain, S. aureus and S. | CR6406 | U.S. Pat. No. 8,460,666 | 26650 |
| | epidermidis | | SEQ ID NO: 135 | |
| STPH57 | Heavy chain, S. aureus and S. | CR6410 | U.S. Pat. No. 8,460,666 | 26651 |
| | epidermidis | | SEQ ID NO: 137 | |
| STPH58 | Heavy chain, S. aureus and S. | CR6446 | U.S. Pat. No. 8,460,666 | 26652 |
| | epidermidis | | SEQ ID NO: 139 | |
| STPH59 | Heavy chain, S. aureus and S. | CR6450 | U.S. Pat. No. 8,460,666 | 26653 |
| | epidermidis | | SEQ ID NO: 141 | |
| STPH60 | Heavy chain, S. aureus and S. | CR6452 | U.S. Pat. No. 8,460,666 | 26654 |
| | epidermidis | | SEQ ID NO: 143 | |
| STPH61 | Heavy chain, S. aureus and S. | CR6453 | U.S. Pat. No. 8,460,666 | 26655 |
| | epidermidis | | SEQ ID NO: 145 | |
| STPH62 | Heavy chain, S. aureus and S. | CR6464 | U.S. Pat. No. 8,460,666 | 26656 |
| | epidermidis | | SEQ ID NO: 147 | |
| STPH63 | Heavy chain, S. aureus and S. | CR6471 | U.S. Pat. No. 8,460,666 | 26657 |
| | epidermidis | | SEQ ID NO: 149 | |
| STPH64 | Heavy chain, S. aureus and S. | CR6516 | U.S. Pat. No. 8,460,666 | 26658 |
| | epidermidis | | SEQ ID NO: 151 | |
| STPH65 | Heavy chain, S. aureus and S. | CR6517 | U.S. Pat. No. 8,460,666 | 26659 |
| | epidermidis | | SEQ ID NO: 153 | |
| STPH66 | Heavy chain, S. aureus and S. | CR6526 | U.S. Pat. No. 8,460,666 | 26660 |
| | epidermidis | | SEQ ID NO: 155 | |
| STPH67 | Heavy chain, S. aureus and S. | CR6528 | U.S. Pat. No. 8,460,666 | 26661 |
| | epidermidis | | SEQ ID NO: 157 | |
| STPH68 | Heavy chain, S. aureus and S. | CR6531 | U.S. Pat. No. 8,460,666 | 26662 |
| | epidermidis | | SEQ ID NO: 159 | |
| STPH69 | Heavy chain, S. aureus and S. | CR6533 | U.S. Pat. No. 8,460,666 | 26663 |
| | epidermidis | | SEQ ID NO: 161 | |
| STPH70 | Heavy chain, S. aureus and S. | CR6536 | U.S. Pat. No. 8,460,666 | 26664 |
| | epidermidis | | SEQ ID NO: 163 | |
| STPH71 | Heavy chain, S. aureus and S. | CR6537 | U.S. Pat. No. 8,460,666 | 26665 |
| | epidermidis | | SEQ ID NO: 165 | |
| STPH72 | Heavy chain, S. aureus and S. | CR6538 | U.S. Pat. No. 8,460,666 | 26666 |
| | epidermidis | | SEQ ID NO: 167 | |
| STPH73 | Heavy chain, S. aureus and S. | CR6540 | U.S. Pat. No. 8,460,666 | 26667 |
| | epidermidis | | SEQ ID NO: 169 | |
| STPH74 | Heavy chain, S. aureus and S. | CR6544 | U.S. Pat. No. 8,460,666 | 26668 |
| | epidermidis | | SEQ ID NO: 171 | |
| STPH75 | Heavy chain, S. aureus and S. | CR6566 | U.S. Pat. No. 8,460,666 | 26669 |
| | epidermidis | | SEQ ID NO: 173 | |
| STPH76 | Heavy chain, S. aureus and S. | CR6625 | U.S. Pat. No. 8,460,666 | 26670 |
| | epidermidis | | SEQ ID NO: 175 | |
| STPH77 | Heavy chain, S. aureus, Enterococcus | CR5140 | U.S. Pat. No. 8,628,776 | 26671 |
| | | | SEQ ID NO: 395 | |
| STPH78 | Heavy chain, S. aureus, Enterococcus | CR5159 | U.S. Pat. No. 8,628,776 | 26672 |
| | | | SEQ ID NO: 82 | |
| STPH79 | Heavy chain, S. aureus, Enterococcus | CR5179 | U.S. Pat. No. 8,628,776 | 26673 |
| | | | SEQ ID NO: 399 | |
| STPH80 | Heavy chain, S. aureus, Enterococcus | CR6016 | U.S. Pat. No. 8,628,776 | 26674 |
| | | | SEQ ID NO: 88 | |
| STPH81 | Heavy chain, S. aureus, Enterococcus | CR6049 | U.S. Pat. No. 8,628,776 | 26675 |
| | | | SEQ ID NO: 92 | |
| STPH82 | Heavy chain, S. aureus. Enterococcus | CR6071 | U.S. Pat. No. 8,628,776 | 26676 |
| | | | SEQ ID NO: 94 | |
| STPH83 | Heavy chain, S. aureus, Enterococcus | CR6078 | U.S. Pat. No. 8,628,776 | 26677 |
| | | | SEQ ID NO: 96 | |
| STPH84 | Heavy chain, S. aureus, Enterococcus | CR6086 | U.S. Pat. No. 8,628,776 | 26678 |
| | | | SEQ ID NO: 407 | |
| STPH85 | Heavy chain, S. aureus, Enterococcus | CR6089 | U.S. Pat. No. 8,628,776 | 26679 |
| | | | SEQ ID NO: 213 | |
| STPH86 | Heavy chain, S. aureus, Enterococcus | CR6191 | U.S. Pat. No. 8,628,776 | 26680 |
| | | | SEQ ID NO: 411 | |
| STPH87 | Heavy chain, S. aureus, Enterococcus | CR6198 | U.S. Pat. No. 8,628,776 | 26681 |
| | | | SEQ ID NO: 415 | |
| STPH88 | Heavy chain, S. aureus, Enterococcus | CR6242 | U.S. Pat. No. 8,628,776 | 26682 |
| | | | SEQ ID NO: 417 | |
| STPH89 | Heavy chain, S. aureus, Enterococcus | CR6252 | U.S. Pat. No. 8,628,776 | 26683 |
| | | | SEQ ID NO: 100 | |
| STPH90 | Heavy chain, S. aureus, Enterococcus | CR6389 | U.S. Pat. No. 8,628,776 | 26684 |
| | | | SEQ ID NO: 423 | |
| STPH91 | Heavy chain, S. aureus, Enterococcus | CR6402 | U.S. Pat. No. 8,628,776 | 26685 |
| | | | SEQ ID NO: 427 | |
| STPH92 | Heavy chain, S. aureus, Enterococcus | CR6415 | U.S. Pat. No. 8,628,776 | 26686 |
| | | | SEQ ID NO: 431 | |
| STPH93 | Heavy chain, S. aureus, Enterococcus | CR6429 | U.S. Pat. No. 8,628,776 | 26687 |
| | | | SEQ ID NO: 435 | |
| STPH94 | Heavy chain, S. aureus, Enterococcus | CR5140 | U.S. Pat. No. 8,628,776 | 26688 |
| | | | SEQ ID NO: 439 | |
| STPH95 | Heavy chain, S. aureus, Enterococcus | CR5159 | U.S. Pat. No. 8,628,776 | 26689 |
| | | | SEQ ID NO: 102 | |
| STPH96 | Heavy chain, S. aureus, Enterococcus | CR5179 | U.S. Pat. No. 8,628,776 | 26690 |
| | | | SEQ ID NO: 443 | |
| STPH97 | Heavy chain, S. aureus, Enterococcus | CR6016 | U.S. Pat. No. 8,628,776 | 26691 |
| | | | SEQ ID NO: 108 | |
| STPH98 | Heavy chain, S. aureus, Enterococcus | CR6049 | U.S. Pat. No. 8,628,776 | 26692 |
| | | | SEQ ID NO: 112 | |
| STPH99 | Heavy chain, S. aureus, Enterococcus | CR6071 | U.S. Pat. No. 8,628,776 | 26693 |
| | | | SEQ ID NO: 114 | |
| STPH100 | Heavy chain, S. aureus, Enterococcus | CR6078 | U.S. Pat. No. 8,628,776 | 26694 |
| | | | SEQ ID NO: 116 | |
| STPH101 | Heavy chain, S. aureus, Enterococcus | CR6086 | U.S. Pat. No. 8,628,776 | 26695 |
| | | | SEQ ID NO: 451 | |
| STPH102 | Heavy chain, S. aureus, Enterococcus | CR6089 | U.S. Pat. No. 8,628,776 | 26696 |
| | | | SEQ ID NO: 217 | |
| STPH103 | Heavy chain, S. aureus, Enterococcus | CR6191 | U.S. Pat. No. 8,628,776 | 26697 |
| | | | SEQ ID NO: 455 | |
| STPH104 | Heavy chain, S. aureus, Enterococcus | CR6198 | U.S. Pat. No. 8,628,776 | 26698 |
| | | | SEQ ID NO: 459 | |
| STPH105 | Heavy chain, S. aureus, Enterococcus | CR6242 | U.S. Pat. No. 8,628,776 | 26699 |
| | | | SEQ ID NO: 461 | |
| STPH106 | Heavy chain, S. aureus, Enterococcus | CR6252 | U.S. Pat. No. 8,628,776 | 26700 |
| | | | SEQ ID NO: 120 | |
| STPH107 | Heavy chain, S. aureus, Enterococcus | CR6389 | U.S. Pat. No. 8,628,776 | 26701 |
| | | | SEQ ID NO: 467 | |
| STPH108 | Heavy chain, S. aureus, Enterococcus | CR6402 | U.S. Pat. No. 8,628,776 | 26702 |
| | | | SEQ ID NO: 471 | |
| STPH109 | Heavy chain, S. aureus, Enterococcus | CR6415 | U.S. Pat. No. 8,628,776 | 26703 |
| | | | SEQ ID NO: 475 | |
| STPH110 | Heavy chain, S. aureus, Enterococcus | CR6429 | U.S. Pat. No. 8,628,776 | 26704 |
| | | | SEQ ID NO: 479 | |
| STPH111 | Heavy chain, S. aureus, S. epidermidis, | F1 antibody | U.S. Pat. No. 8,617,556 | 26705 |
| | S. caprae, S. saprophyticus, S. capitis, or | variant | SEQ ID NO: 7 | |
| | methicillin-resistant S. aureus (MRSA) | | | |
| STPH112 | Heavy chain, S. aureus, S. epidermidis, | F1 antibody | U.S. Pat. No. 8,617,556 | 26706 |
| | S. caprae, S. saprophyticus, S. capitis, or | variant | SEQ ID NO: 9 | |
| | methicillin-resistant S. aureus (MRSA) | | | |
| STPH113 | Heavy chain, S. aureus, S. epidermidis, | rF1 | U.S. Pat. No. 8,617,556 | 26707 |
| | S. caprae, S. saprophyticus, S. capitis, or | | SEQ ID NO: 55 | |
| | methicillin-resistant S. aureus (MRSA) | | | |
| STPH114 | Heavy chain, S. aureus, S. epidermidis, | rF1 A114C | U.S. Pat. No. 8,617,556 | 26708 |
| | S. caprae, S. saprophyticus, S. capitis, or | | SEQ ID NO: 56 | |
| | methicillin-resistant S. aureus (MRSA) | | | |
| STPH115 | Heavy chain, S. aureus, S. epidermidis, | rF1 | U.S. Pat. No. 8,617,556 | 26709 |
| | S. caprae, S. saprophyticus, S. capitis, or | | SEQ ID NO: 63 | |
| | methicillin-resistant S. aureus (MRSA) | | | |
| STPH116 | Heavy chain, S. aureus, S. epidermidis, | rF1 A114C | U.S. Pat. No. 8,617,556 | 26710 |
| | S. caprae, S. saprophyticus, S. capitis, or | | SEQ ID NO: 62 | |
| | methicillin-resistant S. aureus (MRSA) | | | |
| STPH117 | Light chain | | U.S. Pat. No. 8,609,102 | 26711 |
| | | | SEQ ID NO: 4 | |
| STPH118 | Light chain variable region, S. aureus | | U.S. Pat. No. 8,609,102 | 26712 |
| | | | SEQ ID NO: 8 | |
| STPH119 | Light chain variable region, S. aureus or | SAR279356 | U.S. Pat. No. 7,786,255 | 26713 |
| | S. epidermidis, E. coli, Yersinia | | SEQ ID NO: 2 | |
| | pestis (Y. pestis), Y. entercolitica, | | | |
| | Xanthomnonas axonopodis (X. | | | |
| | axonopodis), Pseudonmonas | | | |
| | fuorescerns (P. | | | |
| | fluorescens), Actinobacillus | | | |
| | actinomycetemcomitans (A. | | | |
| | actinomycetemcomitans), A. | | | |
| | pleuropneumoniae, Ralstonia | | | |
| | solanacearum (R. | | | |
| | solanacearum), Bordetella pertussis (B. | | | |
| | pertussis), B. parapertussis or B. | | | |
| | bronchiseptica | | | |
| STPH120 | Light chain variable region, S. aureus or | SAR279356 | US20110002932 SEQ ID | 26714 |
| | S. epidermidis, E. coli, Yersinia | | NO: 2 | |
| | pestis (Y. pestis), Y. entercolitica, | | | |
| | Xanthomnonas axonopodis (X. | | | |
| | axonopodis), Pseudonmonas | | | |
| | fuorescerns (P. | | | |
| | fluorescens), Actinobacillus | | | |
| | actinomycetemcomitans (A. | | | |
| | actinomycetemcomitans), A. | | | |
| | pleuropneumoniae, Ralstonia | | | |
| | solanacearum (R. | | | |
| | solanacearum), Bordetella pertussis (B. | | | |
| | pertussis), B. parapertussis or B. | | | |
| | bronchiseptica | | | |
| STPH121 | Light chain variable region, S. | 108-1 | U.S. Pat. No. 8,475,798 | 26715 |
| | epidermidis | | SEQ ID NO: 16 | |
| STPH122 | Light chain variable region, S. | 108-36 | U.S. Pat. No. 8,475,798 | 26716 |
| | epidermidis | | SEQ ID NO: 20 | |
| STPH123 | Light chain variable region, S. | 110-15 | U.S. Pat. No. 8,475,798 | 26717 |
| | epidermidis | | SEQ ID NO: 24 | |
| STPH124 | Light chain variable region, S. | 108-1VL- | U.S. Pat. No. 8,475,798 | 26718 |
| | epidermidis | Hu | SEQ ID NO: 27 | |
| STPH125 | Light chain variable region, S. | 108-36VL- | U.S. Pat. No. 8,475,798 | 26719 |
| | epidermidis | Hu | SEQ ID NO: 29 | |
| STPH126 | Light chain variable region, S. | 110-15VL- | U.S. Pat. No. 8,475,798 | 26720 |
| | epidermidis | Hu | SEQ ID NO: 31 | |
| STPH127 | Light chain variable region, | Pagibaximab | U.S. Pat. No. 8,372,958 | 26721 |
| | Staphylococcal sepsis | | SEQ ID NO: 89 | |
| STPH128 | Light chain variable region, | Pagibaximab | U.S. Pat. No. 8,372,958 | 26722 |
| | Staphylococcal sepsis | | SEQ ID NO: 10 | |
| STPH129 | Light chain variable region, | Pagibaximab | U.S. Pat. No. 8,372,958 | 26723 |
| | Staphylococcal sepsis | | SEQ ID NO: 16 | |
| STPH130 | Light chain variable region, | F598 | U.S. Pat. No. 8,912,314 | 26724 |
| | Staphylococci such as S. aureus and S. | | SEQ ID NO: 2 | |
| | epidermidis, E. coli such as E. | | | |
| | coli strains O157:H7 and | | | |
| | CFT073, Yersinia pestis, Yersinia | | | |
| | entercolitica, Xanthomonas axonopodis, | | | |
| | Pseudomonas fluorescens (all of which | | | |
| | are sequenced species with complete | | | |
| | pgaABCD loci), and Actinobacillus | | | |
| | actinomycetemcomitans (AA), | | | |
| | Actinobacillus pleuropneumoniae (Ap), | | | |
| | Ralstonia solanacearum (e.g., megaplasmid | | | |
| | form), Bordetella pertussis, Bordetella | | | |
| | parapertussis and Bordetella | | | |
| | bronchiseptica | | | |
| STPH131 | Light chain variable region, | F628 | U.S. Pat. No. 8,912,314 | 26725 |
| | Staphylococci such as S. aureus and S. | | SEQ ID NO: 4 | |
| | epidermidis, E. coli such as E. | | | |
| | coli strains O157: H7 and | | | |
| | CFT073, Yersinia pestis, Yersinia | | | |
| | entercolitica, Xanthomonas axonopodis, | | | |
| | Pseudomonas fluorescens (all of which | | | |
| | are sequenced species with complete | | | |
| | pgaABCD loci), and Actinobacillus | | | |
| | actinomycetemcomitans (AA), | | | |
| | Actinobacillus pleuropneumoniae (Ap), | | | |
| | Ralstonia solanacearum (e.g., megaplasmid | | | |
| | form), Bordetella pertussis, Bordetella | | | |
| | parapertussis and Bordetella | | | |
| | bronchiseptica | | | |
| STPH132 | Light chain variable region, | F630 | U.S. Pat. No. 8,912,314 | 26726 |
| | Staphylococci such as S. aureus and S. | | SEQ ID NO: 6 | |
| | epidermidis, E. coli such as E. | | | |
| | coli strains O157:H7 and | | | |
| | CFT073, Yersinia pestis, Yersinia | | | |
| | entercolitica, Xanthomonas axonopodis, | | | |
| | Pseudomonas fluorescens (all of which | | | |
| | are sequenced species with complete | | | |
| | pgaABCD loci), and Actinobacillus | | | |
| | actinomycetemcomitans (AA), | | | |
| | Actinobacillus pleuropneumoniae (Ap), | | | |
| | Ralstonia solanacearum (e.g., megaplasmid | | | |
| | form), Bordetella pertussis, Bordetella | | | |
| | parapertussis and Bordetella | | | |
| | bronchiseptica | | | |
| STPH133 | Light chain variable region, | F598 | U.S. Pat. No. 8,912,314 | 26727 |
| | Staphylococci such as S. aureus and S. | | SEQ ID NO: 57 | |
| | epidermidis, E. coli such as E. | | | |
| | coli strains O157:H7 and | | | |
| | CFT073, Yersinia pestis, Yersinia | | | |
| | entercolitica, Xanthomonas axonopodis, | | | |
| | Pseudomonas fluorescens (all of which | | | |
| | are sequenced species with complete | | | |
| | pgaABCD loci), and Actinobacillus | | | |
| | actinomycetemcomitans (AA), | | | |
| | Actinobacillus pleuropneumoniae (Ap), | | | |
| | Ralstonia solanacearum (e.g., megaplasmid | | | |
| | form), Bordetella pertussis, Bordetella | | | |
| | parapertussis and Bordetella | | | |
| | bronchiseptica | | | |
| STPH134 | Light chain variable region, | F630 | U.S. Pat. No. 8,912,314 | 26728 |
| | Staphylococci such as S. aureus and S. | | SEQ ID NO: 60 | |
| | epidermidis, E. coli such as E. | | | |
| | coli strains O157:H7 and | | | |
| | CFT073, Yersinia pestis, Yersinia | | | |
| | entercolitica, Xanthomonas axonopodis, | | | |
| | Pseudomonas fluorescens (all of which | | | |
| | are sequenced species with complete | | | |
| | pgaABCD loci), and Actinobacillus | | | |
| | actinomycetemcomitans (AA), | | | |
| | Actinobacillus pleuropneumoniae (Ap), | | | |
| | Ralstonia solanacearum (e.g., megaplasmid | | | |
| | form), Bordetella pertussis, Bordetella | | | |
| | parapertussis and Bordetella | | | |
| | bronchiseptica | | | |
| STPH135 | Light chain, MRSA, MSSA | 2B2 | U.S. Pat. No. 8,735,554 | 26729 |
| | | | SEQ ID NO: 2 | |
| STPH136 | Light chain. MRSA, MSSA | 2G7 | U.S. Pat. No. 8,735,554 | 26730 |
| | | | SEQ ID NO: 4 | |
| STPH137 | Light chain, MRSA, MSSA | 3B12 | U.S. Pat. No. 8,735,554 | 26731 |
| | | | SEQ ID NO: 6 | |
| STPH138 | Light chain, S. aureus | DF1.1 | U.S. Pat. No. 8,715,673 | 26732 |
| | | | SEQ ID NO: 1 | |
| STPH139 | Light chain, S. aureus | DF1-DF20 | U.S. Pat. No. 8,715,673 | 26733 |
| | | | SEQ ID NO: 3 | |
| STPH140 | Light chain, S. aureus and S. epidermidis | CR2430 | U.S. Pat. No. 8,460,666 | 26734 |
| | | | SEQ ID NO: 32 | |
| STPH141 | Light chain, S. aureus and S. epidermidis | CR5132 | U.S. Pat. No. 8,460,666 | 26735 |
| | | | SEQ ID NO: 34 | |
| STPH142 | Light chain, S. aureus and S. epidermidis | CR5133 | U.S. Pat. No. 8,460,666 | 26736 |
| | | | SEQ ID NO: 36 | |
| STPH143 | Light chain, S. aureus and S. epidermidis | CR6166 | U.S. Pat. No. 8,460,666 | 26737 |
| | | | SEQ ID NO: 177 | |
| STPH144 | Light chain, S. aureus and S. epidermidis | CR6171 | U.S. Pat. No. 8,460,666 | 26738 |
| | | | SEQ ID NO: 179 | |
| STPH145 | Light chain, S. aureus and S. epidermidis | CR6176 | U.S. Pat. No. 8,460,666 | 26739 |
| | | | SEQ ID NO: 181 | |
| STPH146 | Light chain, S. aureus and S. epidermidis | CR6187 | U.S. Pat. No. 8,460,666 | 26740 |
| | | | SEQ ID NO: 183 | |
| STPH147 | Light chain, S. aureus and S. epidermidis | CR6193 | U.S. Pat. No. 8,460,666 | 26741 |
| | | | SEQ ID NO: 185 | |
| STPH148 | Light chain, S. aureus and S. epidermidis | CR6249 | U.S. Pat. No. 8,460,666 | 26742 |
| | | | SEQ ID NO: 187 | |
| STPH149 | Light chain, S. aureus and S. epidermidis | CR6273 | U.S. Pat. No. 8,460,666 | 26743 |
| | | | SEQ ID NO: 189 | |
| STPH150 | Light chain, S. aureus and S. epidermidis | CR6389 | U.S. Pat. No. 8,460,666 | 26744 |
| | | | SEQ ID NO: 191 | |
| STPH151 | Light chain, S. aureus and S. epidermidis | CR6403 | U.S. Pat. No. 8,460,666 | 26745 |
| | | | SEQ ID NO: 193 | |
| STPH152 | Light chain, S. aureus and S. epidermidis | CR6406 | U.S. Pat. No. 8,460,666 | 26746 |
| | | | SEQ ID NO: 195 | |
| STPH153 | Light chain, S. aureus and S. epidermidis | CR6410 | U.S. Pat. No. 8,460,666 | 26747 |
| | | | SEQ ID NO: 197 | |
| STPH154 | Light chain, S. aureus and S. epidermidis | CR6446 | U.S. Pat. No. 8,460,666 | 26748 |
| | | | SEQ ID NO: 199 | |
| STPH155 | Light chain, S. aureus and S. epidermidis | CR6450 | U.S. Pat. No. 8,460,666 | 26749 |
| | | | SEQ ID NO: 201 | |
| STPH156 | Light chain, S. aureus and S. epidermidis | CR6452 | U.S. Pat. No. 8,460,666 | 26750 |
| | | | SEQ ID NO: 203 | |
| STPH157 | Light chain, S. aureus and S. epidermidis | CR6453 | U.S. Pat. No. 8,460,666 | 26751 |
| | | | SEQ ID NO: 205 | |
| STPH158 | Light chain, S. aureus and S. epidermidis | CR6464 | U.S. Pat. No. 8,460,666 | 26752 |
| | | | SEQ ID NO: 207 | |
| STPH159 | Light chain, S. aureus and S. epidermidis | CR6471 | U.S. Pat. No. 8,460,666 | 26753 |
| | | | SEQ ID NO: 209 | |
| STPH160 | Light chain, S. aureus and S. epidermidis | CR6516 | U.S. Pat. No. 8,460,666 | 26754 |
| | | | SEQ ID NO: 211 | |
| STPH161 | Light chain, S. aureus and S. epidermidis | CR6517 | U.S. Pat. No. 8,460,666 | 26755 |
| | | | SEQ ID NO: 213 | |
| STPH162 | Light chain, S. aureus and S. epidermidis | CR6526 | U.S. Pat. No. 8,460,666 | 26756 |
| | | | SEQ ID NO: 215 | |
| STPH163 | Light chain, S. aureus and S. epidermidis | CR6528 | U.S. Pat. No. 8,460,666 | 26757 |
| | | | SEQ ID NO: 217 | |
| STPH164 | Light chain, S. aureus and S. epidermidis | CR6531 | U.S. Pat. No. 8,460,666 | 26758 |
| | | | SEQ ID NO: 219 | |
| STPH165 | Light chain, S. aureus and S. epidermidis | CR6533 | U.S. Pat. No. 8,460,666 | 26759 |
| | | | SEQ ID NO: 221 | |
| STPH166 | Light chain, S. aureus and S. epidermidis | CR6536 | U.S. Pat. No. 8,460,666 | 26760 |
| | | | SEQ ID NO: 223 | |
| STPH167 | Light chain, S. aureus and S. epidermidis | CR6537 | U.S. Pat. No. 8,460,666 | 26761 |
| | | | SEQ ID NO: 225 | |
| STPH168 | Light chain, S. aureus and S. epidermidis | CR6538 | U.S. Pat. No. 8,460,666 | 26762 |
| | | | SEQ ID NO: 227 | |
| STPH169 | Light chain, S. aureus and S. epidermidis | CR6540 | U.S. Pat. No. 8,460,666 | 26763 |
| | | | SEQ ID NO: 229 | |
| STPH170 | Light chain, S. aureus and S. epidermidis | CR6544 | U.S. Pat. No. 8,460,666 | 26764 |
| | | | SEQ ID NO: 231 | |
| STPH171 | Light chain, S. aureus and S. epidermidis | CR6566 | U.S. Pat. No. 8,460,666 | 26765 |
| | | | SEQ ID NO: 233 | |
| STPH172 | Light chain, S. aureus and S. epidermidis | CR6625 | U.S. Pat. No. 8,460,666 | 26766 |
| | | | SEQ ID NO: 235 | |
| STPH173 | Light chain, S. aureus, Enterococcus | CR6157 | U.S. Pat. No. 8,628,776 | 26767 |
| | | | SEQ ID NO: 397 | |
| STPH174 | Light chain, S. aureus, Enterococcus | CR5166 | U.S. Pat. No. 8,628,776 | 26768 |
| | | | SEQ ID NO: 84 | |
| STPH175 | Light chain, S. aureus, Enterococcus | CR5187 | U.S. Pat. No. 8,628,776 | 26769 |
| | | | SEQ ID NO: 86 | |
| STPH176 | Light chain, S. aureus, Enterococcus | CR6043 | U.S. Pat. No. 8,628,776 | 26770 |
| | | | SEQ ID NO: 90 | |
| STPH177 | Light chain, S. aureus, Enterococcus | CR6050 | U.S. Pat. No. 8,628,776 | 26771 |
| | | | SEQ ID NO: 401 | |
| STPH178 | Light chain, S. aureus, Enterococcus | CR6077 | U.S. Pat. No. 8,628,776 | 26772 |
| | | | SEQ ID NO: 403 | |
| STPH179 | Light chain, S. aureus, Enterococcus | CR6079 | U.S. Pat. No. 8,628,776 | 26773 |
| | | | SEQ ID NO: 405 | |
| STPH180 | Light chain, S. aureus, Enterococcus | CR6087 | U.S. Pat. No. 8,628,776 | 26774 |
| | | | SEQ ID NO: 211 | |
| STPH181 | Light chain, S. aureus, Enterococcus | CR6092 | U.S. Pat. No. 8,628,776 | 26775 |
| | | | SEQ ID NO: 409 | |
| STPH182 | Light chain, S. aureus, Enterococcus | CR6195 | U.S. Pat. No. 8,628,776 | 26776 |
| | | | SEQ ID NO: 413 | |
| STPH183 | Light chain, S. aureus, Enterococcus | CR6241 | U.S. Pat. No. 8,628,776 | 26777 |
| | | | SEQ ID NO: 98 | |
| STPH184 | Light chain, S. aureus, Enterococcus | CR6246 | U.S. Pat. No. 8,628,776 | 26778 |
| | | | SEQ ID NO: 419 | |
| STPH185 | Light chain, S. aureus, Enterococcus | CR6388 | U.S. Pat. No. 8,628,776 | 26779 |
| | | | SEQ ID NO: 421 | |
| STPH186 | Light chain, S. aureus, Enterococcus | CR6396 | U.S. Pat. No. 8,628,776 | 26780 |
| | | | SEQ ID NO: 425 | |
| STPH187 | Light chain, S. aureus, Enterococcus | CR6409 | U.S. Pat. No. 8,628,776 | 26781 |
| | | | SEQ ID NO: 429 | |
| STPH188 | Light chain, S. aureus, Enterococcus | CR6421 | U.S. Pat. No. 8,628,776 | 26782 |
| | | | SEQ ID NO: 433 | |
| STPH189 | Light chain, S. aureus, Enterococcus | CR6432 | U.S. Pat. No. 8,628,776 | 26783 |
| | | | SEQ ID NO: 437 | |
| STPH190 | Light chain, S. aureus, Enterococcus | CR5157 | U.S. Pat. No. 8,628,776 | 26784 |
| | | | SEQ ID NO: 441 | |
| STPH191 | Light chain, S. aureus, Enterococcus | CR5166 | U.S. Pat. No. 8,628,776 | 26785 |
| | | | SEQ ID NO: 104 | |
| STPH192 | Light chain, S. aureus, Enterococcus | CR5187 | U.S. Pat. No. 8,628,776 | 26786 |
| | | | SEQ ID NO: 106 | |
| STPH193 | Light chain, S. aureus, Enterococcus | CR6043 | U.S. Pat. No. 8,628,776 | 26787 |
| | | | SEQ ID NO: 110 | |
| STPH194 | Light chain, S. aureus, Enterococcus | CR6050 | U.S. Pat. No. 8,628,776 | 26788 |
| | | | SEQ ID NO: 445 | |
| STPH195 | Light chain, S. aureus, Enterococcus | CR6077 | U.S. Pat. No. 8,628,776 | 26789 |
| | | | SEQ ID NO: 447 | |
| STPH196 | Light chain, S. aureus, Enterococcus | CR6079 | U.S. Pat. No. 8,628,776 | 26790 |
| | | | SEQ ID NO: 449 | |
| STPH197 | Light chain, S. aureus, Enterococcus | CR6087 | U.S. Pat. No. 8,628,776 | 26791 |
| | | | SEQ ID NO: 215 | |
| STPH198 | Light chain, S. aureus, Enterococcus | CR6092 | U.S. Pat. No. 8,628,776 | 26792 |
| | | | SEQ ID NO: 453 | |
| STPH199 | Light chain, S. aureus, Enterococcus | CR6195 | U.S. Pat. No. 8,628,776 | 26793 |
| | | | SEQ ID NO: 457 | |
| STPH200 | Light chain, S. aureus, Enterococcus | CR6241 | U.S. Pat. No. 8,628,776 | 26794 |
| | | | SEQ ID NO: 118 | |
| STPH201 | Light chain, S. aureus, Enterococcus | CR6246 | U.S. Pat. No. 8,628,776 | 26795 |
| | | | SEQ ID NO: 463 | |
| STPH202 | Light chain, S. aureus, Enterococcus | CR6388 | U.S. Pat. No. 8,628,776 | 26796 |
| | | | SEQ ID NO: 465 | |
| STPH203 | Light chain, S. aureus, Enterococcus | CR6396 | U.S. Pat. No. 8,628,776 | 26797 |
| | | | SEQ ID NO: 469 | |
| STPH204 | Light chain, S. aureus, Enterococcus | CR6409 | U.S. Pat. No. 8,628,776 | 26798 |
| | | | SEQ ID NO: 473 | |
| STPH205 | Light chain, S. aureus, Enterococcus | CR6421 | U.S. Pat. No. 8,628,776 | 26799 |
| | | | SEQ ID NO: 477 | |
| STPH206 | Light chain, S. aureus, Enterococcus | CR6432 | U.S. Pat. No. 8,628,776 | 26800 |
| | | | SEQ ID NO: 481 | |
| STPH207 | Light chain, S. aureus, S. epidermidis, S. | F1 antibody | U.S. Pat. No. 8,617,556 | 26801 |
| | caprae, S. saprophyticus, S. capitis, or | variant | SEQ ID NO: 8 | |
| | methicillin-resistant S. aureus (MRSA) | | | |
| STPH208 | Light chain, S. aureus, S. epidermidis, S. | F1 antibody | U.S. Pat. No. 8,617,556 | 26802 |
| | caprae, S. saprophyticus, S. capitis, or | variant | SEQ ID NO: 10 | |
| | methicillin-resistant S. aureus (MRSA) | | | |
| STPH209 | Light chain, S. aureus, S. epidermidis, S. | F1 antibody | U.S. Pat. No. 8,617,556 | 26803 |
| | caprae, S. saprophyticus, S. capitis, or | variant | SEQ ID NO: 11 | |
| | methicillin-resistant S. aureus (MRSA) | | | |
| STPH210 | Light chain, S. aureus, S. epidermidis, S. | rF1 | U.S. Pat. No. 8,617,556 | 26804 |
| | caprae, S. saprophyticus, S. capitis, or | | SEQ ID NO: 57 | |
| | methicillin-resistant S. aureus (MRSA) | | | |
| STPH211 | Light chain, S. aureus, S. epidermidis, S. | rF1 V205C | U.S. Pat. No. 8,617,556 | 26805 |
| | caprae, S. saprophyticus, S. capitis, or | | SEQ ID NO: 58 | |
| | methicillin-resistant S. aureus (MRSA) | | | |
| STPH212 | Light chain, S. aureus, S. epidermidis, S. | rF1 | U.S. Pat. No. 8,617,556 | 26806 |
| | caprae, S. saprophyticus, S. capitis, or | | SEQ ID NO: 64 | |
| | methicillin-resistant S. aureus (MRSA) | | | |
| STPH213 | ScFv, S. aureus and S. epidermidis | SC02-430 | U.S. Pat. No. 8,460,666 | 26807 |
| | | | SEQ ID NO: 20 | |
| STPH214 | ScFv, S. aureus and S. epidermidis | SC05-132 | U.S. Pat. No. 8,460,666 | 26808 |
| | | | SEQ ID NO: 22 | |
| STPH215 | ScFv, S. aureus and S. epidermidis | SC05-133 | U.S. Pat. No. 8,460,666 | 26809 |
| | | | SEQ ID NO: 24 | |
| STPH216 | ScFv, S. aureus, Enterococcus | SC05-140 | U.S. Pat. No. 8,628,776 | 26810 |
| | | | SEQ ID NO: 351 | |
| STPH217 | ScFv, S. aureus, Enterococcus | SC05-157 | U.S. Pat. No. 8,628,776 | 26811 |
| | | | SEQ ID NO: 353 | |
| STPH218 | ScFv, S. aureus, Enterococcus | SC05-159 | U.S. Pat. No. 8,628,776 | 26812 |
| | | | SEQ ID NO: 62 | |
| STPH219 | ScFv, S. aureus, Enterococcus | SC05-166 | U.S. Pat. No. 8,628,776 | 26813 |
| | | | SEQ ID NO: 64 | |
| STPH220 | ScFv, S. aureus, Enterococcus | SC05-179 | U.S. Pat. No. 8,628,776 | 26814 |
| | | | SEQ ID NO: 355 | |
| STPH221 | ScFv, S. aureus, Enterococcus | SC05-187 | U.S. Pat. No. 8,628,776 | 26815 |
| | | | SEQ ID NO: 66 | |
| STPH222 | ScFv, S. aureus, Enterococcus | SC06-016 | U.S. Pat. No. 8,628,776 | 26816 |
| | | | SEQ ID NO: 68 | |
| STPH223 | ScFv, S. aureus, Enterococcus | SC06-043 | U.S. Pat. No. 8,628,776 | 26817 |
| | | | SEQ ID NO: 70 | |
| STPH224 | ScFv, S. aureus, Enterococcus | SC06-049 | U.S. Pat. No. 8,628,776 | 26818 |
| | | | SEQ ID NO: 72 | |
| STPH225 | ScFv, S. aureus, Enterococcus | SC06-050 | U.S. Pat. No. 8,628,776 | 26819 |
| | | | SEQ ID NO: 357 | |
| STPH226 | ScFv, S. aureus, Enterococcus | SC06-071 | U.S. Pat. No. 8,628,776 | 26820 |
| | | | SEQ ID NO: 74 | |
| STPH227 | ScFv, S. aureus, Enterococcus | SC06-077 | U.S. Pat. No. 8,628,776 | 26821 |
| | | | SEQ ID NO: 359 | |
| STPH228 | ScFv, S. aureus, Enterococcus | SC06-078 | U.S. Pat. No. 8,628,776 | 26822 |
| | | | SEQ ID NO: 76 | |
| STPH229 | ScFv, S. aureus, Enterococcus | SC06-079 | U.S. Pat. No. 8,628,776 | 26823 |
| | | | SEQ ID NO: 361 | |
| STPH230 | ScFv, S. aureus, Enterococcus | SC06-086 | U.S. Pat. No. 8,628,776 | 26824 |
| | | | SEQ ID NO: 363 | |
| STPH231 | ScFv, S. aureus, Enterococcus | SC06-087 | U.S. Pat. No. 8,628,776 | 26825 |
| | | | SEQ ID NO: 207 | |
| STPH232 | ScFv, S. aureus, Enterococcus | SC06-089 | U.S. Pat. No. 8,628,776 | 26826 |
| | | | SEQ ID NO: 209 | |
| STPH233 | ScFv, S. aureus, Enterococcus | SC06-092 | U.S. Pat. No. 8,628,776 | 26827 |
| | | | SEQ ID NO: 365 | |
| STPH234 | ScFv, S. aureus, Enterococcus | SC06-191 | U.S. Pat. No. 8,628,776 | 26828 |
| | | | SEQ ID NO: 367 | |
| STPH235 | ScFv, S. aureus, Enterococcus | SC06-195 | U.S. Pat. No. 8,628,776 | 26829 |
| | | | SEQ ID NO: 369 | |
| STPH236 | ScFv, S. aureus, Enterococcus | SC06-198 | U.S. Pat. No. 8,628,776 | 26830 |
| | | | SEQ ID NO: 371 | |
| STPH237 | ScFv, S. aureus, Enterococcus | SC06-241 | U.S. Pat. No. 8,628,776 | 26831 |
| | | | SEQ ID NO: 78 | |
| STPH238 | ScFv, S. aureus, Enterococcus | SC06-242 | U.S. Pat. No. 8,628,776 | 26832 |
| | | | SEQ ID NO: 373 | |
| STPH239 | ScFv, S. aureus, Enterococcus | SC06-246 | U.S. Pat. No. 8,628,776 | 26833 |
| | | | SEQ ID NO: 375 | |
| STPH240 | ScFv, S. aureus, Enterococcus | SC06-252 | U.S. Pat. No. 8,628,776 | 26834 |
| | | | SEQ ID NO: 80 | |
| STPH241 | ScFv, S. aureus, Enterococcus | SC06-388 | U.S. Pat. No. 8,628,776 | 26835 |
| | | | SEQ ID NO: 377 | |
| STPH242 | ScFv, S. aureus, Enterococcus | SC06-389 | U.S. Pat. No. 8,628,776 | 26836 |
| | | | SEQ ID NO: 379 | |
| STPH243 | ScFv, S. aureus, Enterococcus | SC06-396 | U.S. Pat. No. 8,628,776 | 26837 |
| | | | SEQ ID NO: 381 | |
| STPH244 | ScFv, S. aureus, Enterococcus | SC06-402 | U.S. Pat. No. 8,628,776 | 26838 |
| | | | SEQ ID NO: 383 | |
| STPH245 | ScFv, S. aureus, Enterococcus | SC06-409 | U.S. Pat. No. 8,628,776 | 26839 |
| | | | SEQ ID NO: 385 | |
| STPH246 | ScFv, S. aureus, Enterococcus | SC06-415 | U.S. Pat. No. 8,628,776 | 26840 |
| | | | SEQ ID NO: 387 | |
| STPH247 | ScFv, S. aureus, Enterococcus | SC06-421 | U.S. Pat. No. 8,628,776 | 26841 |
| | | | SEQ ID NO: 389 | |
| STPH248 | ScFv, S. aureus, Enterococcus | SC06-429 | U.S. Pat. No. 8,628,776 | 26842 |
| | | | SEQ ID NO: 391 | |
| STPH249 | ScFv, S. aureus, Enterococcus | SC06-432 | U.S. Pat. No. 8,628,776 | 26843 |
| | | | SEQ ID NO: 393 | |

In some embodiments, the payload region of the viral particle comprises one or more nucleic acid sequences, fragments or variants thereof or encodes one or more polypeptides, fragments or variants thereof described in International Publication No. WO2000071585, WO2013162751, WO2015089502, WO2015088346 (e.g., SEQ ID NO: 17), US Pub No. US20030224000, US20080014202, US20140037650 US20140170134, U.S. Pat. No. 8,460,666, the contents of each of which are herein incorporated by reference in their entirety, against Staphylococcus infection.

In some embodiments, the payload region of the viral particle comprises one or more nucleic acid sequences encoding one or more of the payload antibody polypeptides listed in Table 46 against Clostridium tetani (CTET1-CTET57; SEQ ID NO: 26844-26900).

TABLE 46
Antibodies against Clostridium Tetani

| | | | | SEQ |
|---|---|---|---|---|
| Antibody | | Antibody | | ID |
| No. | Description | Name | Reference Information | NO |
| CTET1 | Heavy chain | | Sims, G. P. “Tetanus toxoid specific antibody heavy chain | 26844 |
| | partial | | V-gene sequence”, Unpublished, CNBI Accession # | |
| | | | AAC69189.1 | |
| CTET2 | Heavy chain | F5-20 | Sims, G. P. “Tetanus toxoid specific antibody heavy chain | 26845 |
| | variable region | | V-gene sequence”, Unpublished, CNBI Accession # | |
| | | | AAB50736.1 | |
| CTET3 | Heavy chain | | Larrick, J. W., “Therapeutic human antibodies derived | 26846 |
| | variable region | | from PCR amplification of B-cell variable regions”, | |
| | | | Immunol. Rev. 130, 69-85 (1992), CNBI Accession # | |
| | | | AAB25318.1 | |
| CTET4 | Heavy chain | | de Kruif, J. et al., “Human immunoglobulin repertoires | 26847 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36976.1 | |
| CTET5 | Heavy chain | | de Kruif, J. et al., “Human immunoglobulin repertoires | 26848 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36975.1 | |
| CTET6 | Heavy chain | | de Kruif, J. et al., “Human immunoglobulin repertoires | 26849 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36974.1 | |
| CTET7 | Heavy chain | | de Kruif, J. et al., “Human immunoglobulin repertoires | 26850 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36973.1 | |
| CTET8 | Heavy chain | | de Kruif, J. et al., “Human immunoglobulin repertoires | 26851 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36972.1 | |
| CTET9 | Heavy chain | | de Kruif, J. et al., “Human immunoglobulin repertoires | 26852 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36971.1 | |
| CTET10 | Heavy chain | | de Kruif, J. et al., “Human immunoglobulin repertoires | 26853 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36970.1 | |
| CTET11 | Heavy chain | | de Kruif, J. et al., “Human immunoglobulin repertoires | 26854 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36969.1 | |
| CTET12 | Heavy chain | | de Kruif, J. et al., “Human immunoglobulin repertoires | 26855 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36968.1 | |
| CTET13 | Heavy chain | | de Kruif, J. et al., “Human immunoglobulin repertoires | 26856 |
| | variable region, | | against tetanus toxoid contain a large and diverse fraction | |
| | Human | | of high-affinity promiscuous V(H) genes”, J. Mol, Biol, | |
| | immunoglobulin | | 387 (3), 548-558 (2009), CNBI Accession # ACL36967.1 | |
| CTET14 | Heavy chain | | de Kruif, J. et al., “Human immunoglobulin repertoires | 26857 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36966.1 | |
| CTET15 | Heavy chain | | de Kruif, J. et al., “Human immunoglobulin repertoires | 26858 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36965.1 | |
| CTET16 | Heavy chain | | de Kruif, J. et al., “Human immunoglobulin repertoires | 26859 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36964.1 | |
| CTET17 | Heavy chain | | de Kruif, J. et al., “Human immunoglobulin repertoires | 26860 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36963.1 | |
| CTET18 | Heavy chain | | de Kruif, J. et al., “Human immunoglobulin repertoires | 26861 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36962.1 | |
| CTET19 | Heavy chain | | de Kruif, J. et al., “Human immunoglobulin repertoires | 26862 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36961.1 | |
| CTET20 | Heavy chain | | de Kruif, J. et al., “Human immunoglobulin repertoires | 26863 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36960.1 | |
| CTET21 | Heavy chain | | de Kruif, J. et al., “Human immunoglobulin repertoires | 26864 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36958.1 | |
| CTET22 | Heavy chain | | de Kruif, J. et al., “Human immunoglobulin repertoires | 26865 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36959.1 | |
| CTET23 | Heavy chain | | de Kruif, J. et al., “Human immunoglobulin repertoires | 26866 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36957.1 | |
| CTET24 | Heavy chain | | de Kruif, J. et al., “Human immunoglobulin repertoires | 26867 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36956.1 | |
| CTET25 | Heavy chain | | de Kruif, J. et al., “Human immunoglobulin repertoires | 26868 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36955.1 | |
| CTET26 | Heavy chain | | de Kruif, J. et al., “Human immunoglobulin repertoires | 26869 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36954.1 | |
| CTET27 | Heavy chain | | de Kruif, J. et al., “Human immunoglobulin repertoires | 26870 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36953.1 | |
| CTET28 | Heavy chain | | de Kruif, J, et al., “Human immunoglobulin repertoires | 26871 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36952.1 | |
| CTET29 | Heavy chain | | de Kruif, J. et al., “Human immunoglobulin repertoires | 26872 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36951.1 | |
| CTET30 | Heavy chain | | de Kruif, J. et al., “Human immunoglobulin repertoires | 26873 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36950.1 | |
| CTET31 | Heavy chain | | de Kruif, J. et al., “Human immunoglobulin repertoires | 26874 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36949.1 | |
| CTET32 | Heavy chain | | de Kruif, J. et al., “Human immunoglobulin repertoires | 26875 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36948.1 | |
| CTET33 | Heavy chain | | de Kruif, J. et al., “Human immunoglobulin repertoires | 26876 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36947.1 | |
| CTET34 | Heavy chain | | de Kruif, J, et al., “Human immunoglobulin repertoires | 26877 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36946.1 | |
| CTET35 | Heavy chain | | de Kruif, J. et al., “Human immunoglobulin repertoires | 26878 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36924.1 | |
| CTET36 | Heavy chain | | de Kruif, J. et al., “Human immunoglobulin repertoires | 26879 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36925.1 | |
| CTET37 | Heavy chain | | de Kruif, J. et al., “Human immunoglobulin repertoires | 26880 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36926.1 | |
| CTET38 | Heavy chain | | de Kruif, J. et al., “Human immunoglobulin repertoires | 26881 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36927.1 | |
| CTET39 | Heavy chain | | de Kruif, J. et al., “Human immunoglobulin repertoires | 26882 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36928.1 | |
| CTET40 | Heavy chain | | de Kruif, J. et al., “Human immunoglobulin repertoires | 26883 |
| | variable region, | | against tetanus toxoid contain a large and diverse fraction | |
| | Human | | of high-affinity promiscuous V(H) genes”, J, Mol. Biol. | |
| | immunoglobulin | | 387 (3), 548-558 (2009), CNBI Accession # ACL36929.1 | |
| CTET41 | Heavy chain | | de Kruif, J. et al., “Human immunoglobulin repertoires | 26884 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36930.1 | |
| CTET42 | Heavy chain | | de Kruif, J. et al., “Human immunoglobulin repertoires | 26885 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36931.1 | |
| CTET43 | Heavy chain | | de Kruif, J. et al., “Human immunoglobulin repertoires | 26886 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36932.1 | |
| CTET44 | Heavy chain | | de Kruif, J. et al., “Human immunoglobulin repertoires | 26887 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36933.1 | |
| CTET45 | Heavy chain | | de Kruif, J. et al., “Human immunoglobulin repertoires | 26888 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36934.1 | |
| CTET46 | Heavy chain | | de Kruif, J. et al., “Human immunoglobulin repertoires | 26889 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36935.1 | |
| CTET47 | Heavy chain | | de Kruif, J. et al., “Human immunoglobulin repertoires | 26890 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36936.1 | |
| CTET48 | Heavy chain | | e Kruif, J. et al., “Human immunoglobulin repertoires | 26891 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36937.1 | |
| CTET49 | Heavy chain | | e Kruif, J. et al., “Human immunoglobulin repertoires | 26892 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36938.1 | |
| CTET50 | Heavy chain | | e Kruif, J. et al., “Human immunoglobulin repertoires | 26893 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36939.1 | |
| CTET51 | Heavy chain | | e Kruif, J. et al., “Human immunoglobulin repertoires | 26894 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36940.1 | |
| CTET52 | Heavy chain | | e Kruif, J. et al., “Human immunoglobulin repertoires | 26895 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36941.1 | |
| CTET53 | Heavy chain | | e Kruif, J. et al., “Human immunoglobulin repertoires | 26896 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36943.1 | |
| CTET54 | Heavy chain | | e Kruif, J. et al., “Human immunoglobulin repertoires | 26897 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36942.1 | |
| CTET55 | Heavy chain | | e Kruif, J. et al., “Human immunoglobulin repertoires | 26898 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36944.1 | |
| CTET56 | Heavy chain | | e Kruif, J. et al., “Human immunoglobulin repertoires | 26899 |
| | variable region, | | against tetanus toxoid contain a large and diverse fraction | |
| | Human | | of high-affinity promiscuous V(H) genes”, J. Mol. Biol. | |
| | immunoglobulin | | 387 (3), 548-558 (2009), CNBI Accession # ACL36945.1 | |
| CTET57 | Light chain | | Larrick, J. W.. “Therapeutic human antibodies derived | 26900 |
| | variable region | | from PCR amplification of B-cell variable regions”, | |
| | | | Immunol. Rev. 130, 69-85 (1992), CNBI Accession # | |
| | | | AAB25319.1 | |

In some embodiments, the payload region of the viral particle comprises one or more nucleic acid sequences encoding one or more of the payload antibody polypeptides listed in Table 47 against Bordetella Pertussis and/or Bordetella Parapertussis (BORT1-BORT25; SEQ. ID NO: 26901-26925).

TABLE 47
Antibodies against Bordetella Pertussis and Bordetella Parapertussis

| | | | | SEQ |
|---|---|---|---|---|
| Antibody | | Antibody | | ID |
| No. | Description | Name | Reference information | NO |
| BORT1 | Heavy chain | 42.1 1.D4 | WO2014160098 SEQ ID NO: 47 | 26901 |
| BORT2 | Heavy chain | 42.12.G2 | WO2014160098 SEQ ID NO: 51 | 26902 |
| BORT3 | Heavy chain | 42.12.A12 | WO2014160098 SEQ ID NO: 55 | 26903 |
| BORT4 | Heavy chain | 42.12.A9 | WO2014160098 SEQ ID NO: 59 | 26904 |
| BORT5 | Heavy chain | 42.18.E12 | WO2014160098 SEQ ID NO: 63 | 26905 |
| BORT6 | Heavy chain | 55.12.A8 | WO2014160098 SEQ ID NO: 67 | 26906 |
| BORT7 | Heavy chain | 55.15.H5 | WO2014160098 SEQ ID NO: 71 | 26907 |
| BORT8 | Heavy chain | 55.17.D8 | WO2014160098 SEQ ID NO: 75 | 26908 |
| BORT9 | Heavy chain | 55.22.E7 | WO2014160098 SEQ ID NO: 79 | 26909 |
| BORT10 | Light chain | 42,1 1.D4 | WO2014160098 SEQ ID NO: 49 | 26910 |
| BORT11 | Light chain | 42.12.G2 | WO2014160098 SEQ ID NO: 53 | 26911 |
| BORT12 | Light chain | 42.12.A12 | WO2014160098 SEQ ID NO: 57 | 26912 |
| BORT13 | Light chain | 42.12.A9 | WO2014160098 SEQ ID NO: 61 | 26913 |
| BORT14 | Light chain | 42.18.E12 | WO2014160098 SEQ ID NO: 65 | 26914 |
| BORT15 | Light chain | 55.12.A8 | WO2014160098 SEQ ID NO: 69 | 26915 |
| BORT16 | Light chain | 55.15.H5 | WO2014160098 SEQ ID NO: 73 | 26916 |
| BORT17 | Light chain | 55.17.D8 | WO2014160098 SEQ ID NO: 77 | 26917 |
| BORT18 | Light chain | 55.22.E7 | WO2014160098 SEQ ID NO: 81 | 26918 |
| BORT19 | Single chain variable | | Hussein, A. H. et al. “Construction and | 26919 |
| | fragment antibody type | | characterization of single-chain variable | |
| | 1 a1, single chain | | fragment antibodies directed against the | |
| | variable region | | Bordetella pertussis surface adhesins | |
| | | | filamentous hemagglutinin and pertactin” | |
| | | | Infect. Immun. 75 (11), 5476-5482 (2007), | |
| | | | NCBI Accession # ABB13478.1 | |
| BORT20 | Single chain variable | | Hussein, A. H. et al. “Construction and | 26920 |
| | fragment antibody type | | characterization of single-chain variable | |
| | 18 a18, single chain | | fragment antibodies directed against the | |
| | variable region | | Bordetella pertussis surface adhesins | |
| | | | filamentous hemagglutinin and pertactin” | |
| | | | Infect. Immun. 75 (11), 5476-5482 (2007), | |
| | | | NCBI Accession # ABB13483.1 | |
| BORT21 | Single chain variable | | Hussein, A. H, el al. “Construction and | 26921 |
| | fragment antibody type | | characterization of single-chain variable | |
| | 2 a2, single chain | | fragment antibodies directed against the | |
| | variable region | | Bordetella pertussis surface adhesins | |
| | | | filamentous hemagglutinin and pertactin” | |
| | | | Infect. Immun. 75 (11), 5476-5482 (2007), | |
| | | | NCBI Accession # ABB13479.1 | |
| BORT22 | Single chain variable | | Hussein, A. H. et al. “Construction and | 26922 |
| | fragment antibody type | | characterization of single-chain variable | |
| | 4 b4, single chain | | fragment antibodies directed against the | |
| | variable region | | Bordetella pertussis surface adhesins | |
| | | | filamentous hemagglutinin and pertactin” | |
| | | | Infect. Immun. 75 (11), 5476-5482 (2007), | |
| | | | NCBI Accession # ABB13480.1 | |
| BORT23 | Single chain variable | | Hussein, A. H. et al. “Construction and | 26923 |
| | fragment antibody type | | characterization of single-chain variable | |
| | 5 c5, single chain | | fragment antibodies directed against the | |
| | variable region | | Bordetella pertussis surface adhesins | |
| | | | filamentous hemagglutinin and pertactin” | |
| | | | Infect. Immun. 75 (11), 5476-5482 (2007), | |
| | | | NCBI Accession # ABB13481.1 | |
| BORT24 | Single chain variable | | Hussein, A. H. et al. “Construction and | 26924 |
| | fragment antibody type | | characterization of single-chain variable | |
| | 6 d6, single chain | | fragment antibodies directed against the | |
| | variable region | | Bordetella pertussis surface adhesins | |
| | | | filamentous hemagglutinin and pertactin” | |
| | | | Infect. Immun. 75 (11), 5476-5482 (2007), | |
| | | | NCBI Accession # ABB13482.1 | |
| BORT25 | Single chain variable | | Hussein, A. H. et al. “Construction and | 26925 |
| | fragment antibody type | | characterization of single-chain variable | |
| | 7 e, single chain | | fragment antibodies directed against the | |
| | variable region | | Bordetella pertussis surface adhesins | |
| | | | filamentous hemagglutinin and pertactin” | |
| | | | Infect. Immun. 75 (11), 5476-5482 (2007), | |
| | | | NCBI Accession # ABB13484.1 | |

In some embodiments, the payload region of the viral particle comprises one or more nucleic acid sequences encoding one or more of the payload antibody polypeptides listed in Table 48 against Mycobacteria (MYCO1-MYCO16; SU) ID NO: 26926-26941).

TABLE 48
Antibodies against Mycobacteria

| | | | | SEQ |
|---|---|---|---|---|
| Antibody | | Antibody | | ID |
| No. | Description | Name | Reference Information | NO |
| MYCO1 | Autod2 Single-chain variable | | Berger et al., Microbes Infect, 9 | 26926 |
| | fragment antibody, Tb antibody, anti- | | (8), 963-970 (2007), NCBI | |
| | neutrophil cytoplasmic antibodies | | Accession # ABI81486.1 | |
| | cross-react with mycobacterium avium | | | |
| | subsp. Paratuberculosis antigens | | | |
| MYCO2 | autoh1 single-chain variable fragment | | Berger et al., Microbes Infect. 9 | 26927 |
| | antibody, Tb antibody, anti-neutrophil | | (8), 963-970 (2007), NCBI | |
| | cytoplasmic antibodies cross-react | | Accession # ABI81485.1 | |
| | with mycobacterium avium subsp. | | | |
| | Paratuberculosis antigens, | | | |
| MYCO3 | Heavy chain constant region, | moG2a/ | US20130309237 SEQ ID NO: | 26928 |
| | Mycobacteria | moG2afull | 10 | |
| MYCO4 | Heavy chain constant region, | hG1mG2a | US20130309237 SEQ ID NO: | 26929 |
| | Mycobacteria | | 11 | |
| MYCO5 | Heavy chain constant region, | hG3mG2a | US20130309237 SEQ ID NO: | 26930 |
| | Mycobacteria | | 12 | |
| MYCO6 | Heavy chain constant region, | huG1full | US20130309237 SEQ ID NO: | 26931 |
| | Mycobacteria | | 13 | |
| MYCO7 | Heavy chain constant region, | huG3full | US20130309237 SEQ ID NO: | 26932 |
| | Mycobacteria | | 14 | |
| MYCO8 | Heavy chain variable region, | 2F12 IgGs | US20130309237 SEQ ID NO: | 26933 |
| | Mycobacteria | | 15 | |
| MYCO9 | Heavy chain variable region, | 2F12 IgGs | US20130309237 SEQ ID NO: | 26934 |
| | Mycobacteria | | 18 | |
| MYCO10 | Heavy chain variable region, partial | 16a1 | Al-sayyed et al., Tuberculosis | 26935 |
| | sequence, Tb antibody, mouse | | (Edinb) 87 (6), 489-497 (2007), | |
| | monoclonal mpt51 | | NCBI Accession # ABS20005.1 | |
| MYCO11 | Light chain constant region, | HuCK | US20130309237 SEQ ID NO: | 26936 |
| | Mycobacteria | | 16 | |
| MYCO12 | Light chain variable region, | MoCK | US20130309237 SEQ ID NO: | 26937 |
| | Mycobacteria | | 17 | |
| MYCO13 | Light chain variable region, partial | 16a1 | Al-sayyed el al., Tuberculosis | 26938 |
| | sequence, Tb antibody, mouse | | (Edinb) 87 (6), 489-497 (2007), | |
| | monoclonal mpt51 | | NCBI Accession # ABS20006.1 | |
| MYC014 | Scfv, Tb antibody, an engineered | | US20060229438 SEQ ID NO: 3 | 26939 |
| | single chain antibody | | | |
| MYC015 | Scfv, Tb antibody, an engineered | | US20060229438 SEQ ID NO: 4 | 26940 |
| | single chain antibody | | | |
| MYC016 | Scfv, Tb antibody, an engineered | | US20060229438 SEQ ID NO: 2 | 26941 |
| | single chain antibody | | | |

In some embodiments, the payload region of the viral particle comprises one or more nucleic acid sequences encoding one or more of the payload antibody polypeptides listed in Table 49 against Francisella tularensis (FRAN1-FRAN16; SEQ ID NO: 26942-26957).

TABLE 49
Antibodies against Francisella Tularensis

| | | | | SEQ |
|---|---|---|---|---|
| Antibody | | Antibody | | ID |
| No. | Description | Name | Reference Information | NO |
| FRAN1 | Chain H | Ab-52 | Rynkiewicz, M. J. et al., “Structural Analysis of a Protective | 26942 |
| | | | Epitope of the Francisella tularensis O-Polysaccharide”, | |
| | | | Biochemistry- 51 (28), 5684-5694 (2012), NCBI Accession # | |
| | | | 3UJT_H | |
| FRAN2 | Chain H | N62 | Lu, Z., et al., “The binding sites of monoclonal antibodies to | 26943 |
| | | | the non-reducing end of Francisella tularensis O-antigen | |
| | | | accommodate mainly the terminal saccharide”, Immunology | |
| | | | 140 (3), 374-389 (2013), NCBI Accession # 4KPH_H | |
| FRAN3 | Chain H | Ab64 | Lu, Z. et al., “B-cell epitopes in GroEL of Francisella | 26944 |
| | | | tularensis”, PLoS ONE 9 (6), E99847 (2014), NCBI | |
| | | | Accession # 4PB9_H | |
| FRAN4 | Chain H | Ab53 | Lu, Z, et al., “B-cell epitopes in GroEL of Francisella | 26945 |
| | | | tularensis”, PLoS ONE 9 (6), E99847 (2014), NCBI | |
| | | | Accession # 4PB0_H | |
| FRAN5 | Chain H | N203 | Lu, Z. et al., “Functional and Structural Characterization of | 26946 |
| | | | Francisella tularensis O-Antigen Antibodies at the Low End | |
| | | | of Antigen Reactivity”, Monoclonal Antib Immunodiagn | |
| | | | Immunother 33 (4), 235-245 (2014), NCBI Accession # | |
| | | | 4OTX_H | |
| FRAN6 | Chain I | Ab-52 | Rynkiewicz, M. J., et al., “Structural Analysis of a Protective | 26947 |
| | | | Epitope of the Francisella tularensis O-Polysaccharide”, | |
| | | | Biochemistry 51 (28), 5684-5694 (2012), NCBI Accession # | |
| | | | 3UJT_I | |
| FRAN7 | Chain I | N62 | Lu, Z., et al., “The binding sites of monoclonal antibodies to | 26948 |
| | | | the non-reducing end of Francisella tularensis O-antigen | |
| | | | accommodate mainly the terminal saccharide”, Immunology | |
| | | | 140 (3), 374-389 (2013), NCBI Accession # 4KPH_I | |
| FRAN8 | Chain I | N203 | Lu, Z. et al., “Functional and Structural Characterization of | 26949 |
| | | | Francisella tularensis O-Antigen Antibodies at the Low End | |
| | | | of Antigen Reactivity”, Monoclonal Antib Immunodiagn | |
| | | | Immunother 33 (4), 235-245 (2014), NCBI Accession # | |
| | | | 4OTX_I | |
| FRAN9 | Chain L | Ab-52 | Rynkiewicz, M. J., et al., “Structural Analysis of a Protective | 26950 |
| | | | Epitope of the Francisella tularensis O-Polysaccharide”, | |
| | | | Biochemistry 51 (28), 5684-5694 (2012), NCBI Accession # | |
| | | | 3UJT_L | |
| FRAN10 | Chain L | N62 | Lu, Z., et al., “The binding sites of monoclonal antibodies to | 26951 |
| | | | the non-reducing end of Francisella tularensis O-antigen | |
| | | | accommodate mainly the terminal saccharide”, Immunology | |
| | | | 140 (3), 374-389 (2013), NCBI Accession # 4KPH_L | |
| FRAN11 | Chain L | Ab64 | Lu, Z. et al., “B-cell epitopes in GroEL of Francisella | 26952 |
| | | | tularensis”, PLoS ONE 9 (6), E99847 (2014), NCBI | |
| | | | Accession # 4PB9_L | |
| FRAN12 | Chain L | Ab53 | Lu, Z. et al., “B-cell epitopes in GroEL of Francisella | 26953 |
| | | | tularensis”, PLoS ONE 9 (6), E99847 (2014), NCBI | |
| | | | Accession # 4PB0_L | |
| FRAN13 | Chain L | N203 | Lu, Z, et al., “Functional and Structural Characterization of | 26954 |
| | | | Francisella tularensis O-Antigen Antibodies at the Low End | |
| | | | of Antigen Reactivity”, Monoclonal Antib Immunodiagn | |
| | | | Immunother 33 (4), 235-245 (2014), NCBI Accession # | |
| | | | 4OTX_L | |
| FRAN14 | Chain M | Ab-52 | Rynkiewicz, M. J. et al., “Structural Analysis of a Protective | 26955 |
| | | | Epitope of the Francisella tularensis O-Polysaccharide”, | |
| | | | Biochemistry 51 (28), 5684-5694 (2012), NCBI Accession # | |
| | | | 3UJT_M | |
| FRAN15 | Chain M | N62 | Lu, Z., et al., “The binding sites of monoclonal antibodies to | 26956 |
| | | | the non-reducing end of Francisella tularensis O-antigen | |
| | | | accommodate mainly the terminal saccharide”, Immunology | |
| | | | 140 (3), 374-389 (2013), NCBI Accession # 4KPH_M | |
| FRAN16 | Chain M | N203 | Lu, Z, et al., “Functional and Structural Characterization of | 26957 |
| | | | Francisella tularensis O-Antigen Antibodies at the Low End | |
| | | | of Antigen Reactivity”, Monoclonal Antib Immunodiagn | |
| | | | Immunother 33 (4), 235-245 (2014), NCBI Accession # | |
| | | | 4OTX_M | |

In some embodiments, the payload region of the viral particle comprises one or more nucleic acid sequences encoding one or more of the payload antibody polypeptides listed in Table 50 against Bacteria (BACI1-BACI24; SEQ ID NO: 26958-26981).

TABLE 50
Antibodies against Bacteria

| | | | | SEQ |
|---|---|---|---|---|
| Antibody | | | | ID |
| No. | Description | Antibody Name | Reference Information | NO |
| BACI1 | Heavy chain variable | | US20080038266 SEQ ID NO: 1 | 26958 |
| | region, Enterococcus | | | |
| | faecium, Enterococcus | | | |
| | faecalis, Clostridium | | | |
| | difficile | | | |
| BACI2 | Heavy chain variable | Naid60 | US20060073139 SEQ ID NO: 5 | 26959 |
| | region, Neisseria | | | |
| | meningitidis, | | | |
| BACI3 | Heavy chain, Neisseria | | Fernandez de Cossio, M. E., et al. | 26960 |
| | meningitidis, | | “Human monoclonal antibodies against | |
| | | | an epitope on the class 5c outer | |
| | | | membrane protein common to many | |
| | | | pathogenic strains of Neisseria | |
| | | | meningitidis”, J. Infect. Dis. 166 (6), | |
| | | | 1322-1328 (1992), AAB18935 | |
| BACI4 | Heavy chain, Neisseria | | Fernandez de Cossio, M. E., et al. | 26961 |
| | meningitidis, | | “Human monoclonal antibodies against | |
| | | | an epitope on the class 5c outer | |
| | | | membrane protein common to many | |
| | | | pathogenic strains of Neisseria | |
| | | | meningitidis”, J. Infect. Dis. 166 (6), | |
| | | | 1322-1328 (1992), AAB18934 | |
| BACI5 | Heavy chain, Septic | Edobacomab, | | 26962 |
| | shock, meningococcal | E5, XMMEN- | | |
| | septic shock | 0E5 | | |
| BACI6 | Ig kappa chain V-I | | Goni, F. and Frangione, B., “Amino acid | 26963 |
| | region WEA, | | sequence of the Fv region of a human | |
| | Klebsiella bacteria | | monoclonal IgM (protein WEA) with | |
| | | | antibody activity against 3,4-pyruvylated | |
| | | | galactose in Klebsiella polysaccharides | |
| | | | K30 and K33”, Proc. Natl. Acad. Sci. | |
| | | | U.S.A. 80 (15), 4837-4841 (1983). | |
| | | | P01610 | |
| BACI7 | Ig kappa chain V-I | | Goni, F. and Frangione, B., “Amino acid | 26964 |
| | region WEA, | | sequence of the Fv region of a human | |
| | Klebsiella bacteria | | monoclonal IgM (protein WEA) with | |
| | | | antibody activity against 3,4-pyruvylated | |
| | | | galactose in Klebsiella polysaccharides | |
| | | | K30 and K33”, Proc. Natl. Acad. Sci. | |
| | | | U.S.A. 80 (15), 4837-4841 (1983), | |
| | | | P01763 | |
| BACI8 | Light chain variable | | US20080038266 SEQ ID NO: 16 | 26965 |
| | region, Enterococcus | | | |
| | faecium, Enterococcus | | | |
| | faecalis, Clostridium | | | |
| | difficile | | | |
| BACI9 | Light chain variable | Naid60 | US20060073139 SEQ ID NO: 6 | 26966 |
| | region, Neisseria | | | |
| | meningitidis | | | |
| BACI10 | Light chain, Septic | Edobacomab, | | 26967 |
| | shock, meningococcal | E5, XMMEN- | | |
| | septic shock, | 0E5 | | |
| BACI11 | scFv antibody, Anti- | | Zou, N., et al. “Human Single-Chain Fv | 26968 |
| | Burkholderia mallei | | Antibodies against Burkholderia mallei | |
| | | | and Burkholderia pseudomallei”, | |
| | | | unpublished, NCBI Accession # | |
| | | | ABI97022.1 | |
| BACI12 | scFv antibody, Anti- | | Zou, N., et al. “Human Single-Chain Fv | 26969 |
| | Burkholderia mallei | | Antibodies against Burkholderia mallei | |
| | | | and Burkholderia pseudomallei”, | |
| | | | unpublished, NCBI Accession # | |
| | | | ABI97023.1 | |
| BACI13 | scFv antibody, Anti- | | Zou, N., et al. “Human Single-Chain Fv | 26970 |
| | Burkholderia mallei | | Antibodies against Burkholderia mallei | |
| | | | and Burkholderia pseudomallei”, | |
| | | | unpublished, NCBI Accession # | |
| | | | ABI97021.1 | |
| BACI14 | scFv antibody, Anti- | | Zou, N., et al. “Human Single-Chain Fv | 26971 |
| | Burkholderia mallei | | Antibodies against Burkholderia mallei | |
| | | | and Burkholderia pseudomallei”, | |
| | | | unpublished, NCBI Accession # | |
| | | | ABI97018.1 | |
| BACI15 | scFv antibody, Anti- | | Zou, N., et al. “Human Single-Chain Fv | 26972 |
| | Burkholderia mallei | | Antibodies against Burkholderia mallei | |
| | | | and Burkholderia pseudomallei”, | |
| | | | unpublished, NCBI Accession # | |
| | | | ABI97024.1 | |
| BACI16 | scFv antibody, Anti- | | Zou, N., et al. “Human Single-Chain Fv | 26973 |
| | Burkholderia mallei | | Antibodies against Burkholderia mallei | |
| | | | and Burkholderia pseudomallei”, | |
| | | | unpublished, NCBI Accession # | |
| | | | ACZ65033.1 | |
| BACI17 | scFv antibody, Anti- | | Zou, N., et al. “Human Single-Chain Fv | 26974 |
| | Burkholderia mallei | | Antibodies against Burkholderia mallei | |
| | | | and Burkholderia pseudomallei”, | |
| | | | unpublished, NCBI Accession # | |
| | | | ACZ65032.1 | |
| BACI18 | scFv antibody, Anti- | | Zou, N., et al. “Human Single-Chain Fv | 26975 |
| | Burkholderia mallei | | Antibodies against Burkholderia mallei | |
| | | | and Burkholderia pseudomallei”, | |
| | | | unpublished, NCBI Accession # | |
| | | | ACZ65031.1 | |
| BACI19 | scFv antibody, Anti- | | Zou, N., et al. “Human Single-Chain Fv | 26976 |
| | Burkholderia mallei | | Antibodies against Burkholderia mallei | |
| | | | and Burkholderia pseudomallei”, | |
| | | | unpublished, NCBI Accession # | |
| | | | ACZ65030.1 | |
| BACI20 | scFv antibody, Anti- | | Zou, N., et al. “Human Single-Chain. Fv | 26977 |
| | Burkholderia mallei | | Antibodies against Burkholderia mallei | |
| | | | and Burkholderia pseudomallei”, | |
| | | | unpublished, NCBI Accession # | |
| | | | ACZ65029.1 | |
| BACI21 | scFv antibody, Anti- | | Zou, N., et al. “Human Single-Chain Fv | 26978 |
| | Burkholderia mallei | | Antibodies against Burkholderia mallei | |
| | | | and Burkholderia pseudomallei”, | |
| | | | unpublished, NCBI Accession # | |
| | | | ACZ65028.1 | |
| BAC122 | scFv antibody, Anti- | | Zou, N,, et al. “Human Single-Chain Fv | 26979 |
| | Burkholderia mallei | | Antibodies against Burkholderia mallei | |
| | | | and Burkholderia pseudomallei”, | |
| | | | unpublished, NCBI Accession # | |
| | | | AB197020.1 | |
| BACI23 | scFv antibody, Anti- | | Zou, N., et al. “Human Single-Chain Fv | 26980 |
| | Burkholderia mallei | | Antibodies against Burkholderia mallei | |
| | | | and Burkholderia pseudomallei”, | |
| | | | unpublished, NCBI Accession # | |
| | | | ABI97019.1 | |
| BACI24 | Single chain variable, | CB515 | LaRocca, T. J., et al. “Bactericidal action | 26981 |
| | Borrelia, | | of a complement-independent relapsing | |
| | | | fever Borrelia resides in its variable | |
| | | | region”, J. Immunol. 180 (9), 6222-6228 | |
| | | | (2008), NCBI Accession # ABV22509 | |

In some embodiments, the payload region of the viral particle comprises one or more nucleic acid sequences, fragments or variants encoding Doxorubicin, fragments or variants thereof for treating a disease and/or disorder or preventing a disease and/or disorder. As a non-limiting example, the disease and/or disorder is bacterial infection.

In some embodiments, the payload region of the viral particle comprises one or more nucleic acid sequences encoding one or more of the payload antibody polypeptides listed in Table 51 against Toxoplasma gondii (TOXO1-TOXO2; SEQ ID NO: 26982-26983).

TABLE 51
Antibodies against Toxoplasma gondii

| Antibody | | | | SEQ ID |
|---|---|---|---|---|
| No. | Description | Antibody Name | Reference Information | NO |
| TOXO1 | 4f11e12 | Fab Heavy Chain Variable | Graille, M. et al., J. Mol. Biol. 354 | 26982 |
| | | Region, Surface antigen 1 | (2), 447-458 (2005), NCBI Accession | |
| | | (SAG1) | # 1YNT_D (218aa) | |
| TOXO2 | 4f11e12 | Fab Light Chain Variable | Graille, M. et al., J. Mol, Biol. 354 | 26983 |
| | | Region, Surface antigen 1 | (2), 447-458 (2005), NCBI Accession | |
| | | (SAG1) | # 1YNT_C (213aa) | |

In some embodiments, the payload region of the viral particle comprises one or more nucleic acid sequences encoding one or more of the payload antibody polypeptides listed in Table 52 against Candida Yeast (CAND1; SEQ ID NO: 26984).

TABLE 52
Antibodies against Candida Yeast

| Antibody | | | SEQ ID |
|---|---|---|---|
| No. | Description | Antibody Name | NO |
| CAND1 | Efungumab | Candida | 26984 |

In some embodiments, the payload region of the viral particle comprises one or more nucleic acid sequences encoding one or more of the payload antibody polypeptides listed in Table 53 (HIV1-HIV1601; SEQ ID NO: 26985-28585).

TABLE 53
HIV Antibodies

| | | | | SEQ |
|---|---|---|---|---|
| Antibody | | Antibody | | ID |
| No. | Description | Name | Reference Information | NO |
| HIV1 | 4e10 Antibody | 4e10antibody | NCBI Accession # 4OB5_H (127aa) | 26985 |
| | Germline Precursor 7 | | | |
| | Heavy Chain Fv | | | |
| HIV2 | 4e10 Antibody | 4e10antibody | NCBI Accession # 4OB5_L (114aa) | 26986 |
| | Germline Precursor 7 | | | |
| | Light Chain Fv | | | |
| HIV3 | Antigen Binding | Ch103 | Liao et al., Co-evolution of a broadly | 26987 |
| | Fragment Of Heavy | | neutralizing HIV-1 antibody and founder virus; | |
| | Chain | | Nature 496 (7446), 469-476 (2013), NCBI | |
| | | | Accession # 4JAM_H (226aa) | |
| HIV4 | Antigen Binding | Ch103 | Liao et al., Co-evolution of a broadly | 26988 |
| | Fragment Of Light | | neutralizing HIV-1 antibody and founder virus; | |
| | Chain | | Nature 496 (7446), 469-476 (2013), NCBI | |
| | | | Accession # 4JAM_L (209aa) | |
| HIV5 | Fab Fragment, Heavy | N12-i15 | NCBI Accession # 3QEH_G (232aa) | 26989 |
| | Chain | | | |
| HIV6 | Fab Fragment, Light | N12-i15 | NCBI Accession # 3QEH_H (218aa) | 26990 |
| | Chain | | | |
| HIV7 | Fab Heavy Chain | 35o22 | Huang et al., Broad and potent HIV-1 | 26991 |
| | | | neutralization by a human antibody that binds | |
| | | | the gp41-gp120 interface; Nature 515 (7525), | |
| | | | 138-142 (2014), NCBI Accession # 4TOY_H | |
| | | | (243 aa) | |
| HIV8 | Fab Heavy Chain | 8anc195 | Scharf, L., et al., Cell Rep 7 (3), 785-795 | 26992 |
| | | | (2014), NCBI Accession # 4P9H_H (244aa) | |
| HIV9 | Fab Heavy Chain | B13 | Chen L. et al., Science 326 (5956), 1123-1127 | 26993 |
| | | | (2009), NCBI Accession # 3IDY_B (231aa) | |
| HIV10 | Fab Heavy Chain | Ch58 | Liao et al., vaccine Induction of Antibodies | 26994 |
| | | | against a Structurally Heterogeneous Site of | |
| | | | Immune Pressure within HIV-1 Envelope | |
| | | | Protein Variable region 1 and 2; Immunity 38 | |
| | | | (1), 176-186 (2013), NCBI Accession # | |
| | | | 4HQQ_H (231aa) | |
| HIV11 | Fab Heavy Chain | Ch59 | Liao et al., vaccine Induction of Antibodies | 26995 |
| | | | against a Structurally Heterogeneous Site of | |
| | | | Immune Pressure within HIV-1 Envelope | |
| | | | Protein Variable region 1 and 2; Immunity 38 | |
| | | | (1), 176-186 (2013), NCBI Accession # | |
| | | | 4HPY_H (225aa) | |
| HIV12 | Fab Heavy Chain | E51 | Huang C et al., Proc. Natl, Acad. Sci. U.S.A. | 26996 |
| | | | 101 (9), 2706-2711 (2004), NCBI Accession # | |
| | | | 1RZF_H (235aa) | |
| HIV13 | Fab Heavy Chain | N26-i1 Fab | NCBI Accession # 4FZE_H(232aa) | 26997 |
| HIV14 | Fab Heavy Chain | Pgt145 | McLellan, J. S. et al., Structure of HIV-1 gp120 | 26998 |
| | | | V1 V2 domain with broadly neutralizing | |
| | | | antibody PG9; Nature 480 (7377), 336-343 | |
| | | | (2011), NCBI Accession # 3U1S_H (267aa) | |
| HIV15 | Fab Heavy Chain Of | A32 Fab | NCBI Accession # 3TNM_A (231aa) | 26999 |
| | Human Anti-hiv-1 | | | |
| | Env Antibody A32 | | | |
| HIV16 | Fab Heavy Chain Of | C11 Fab | NCBI Accession # 4FZ8_H (237aa) | 27000 |
| | Human Anti-hiv-1 | | | |
| | Env Antibody C11 | | | |
| HIV17 | Fab Light Chain | 35o22 | Huang et al., Broad and potent HIV-1 | 27001 |
| | | | neutralization by a human antibody that binds | |
| | | | the gp41-gp120 interface; Nature 515 (7525), | |
| | | | 138-142 (2014), NCBI Accession # 4TOY_L | |
| | | | (216aa) | |
| HIV18 | Fab Light Chain | 8anc195 | Scharf, L., et al., Cell Rep 7 (3), 785-795 | 27002 |
| | | | (2014), NCBI Accession # 4P9H_L (215aa) | |
| HIV19 | Fab Light Chain | B13 | Chen L. et al., Science 326 (5956), 1123-1127 | 27003 |
| | | | (2009), NCBI Accession # 3IDY_C (215aa) | |
| HIV20 | Fab Light Chain | Ch58 | Liao et al., vaccine Induction of Antibodies | 27004 |
| | | | against a Structurally Heterogeneous Site of | |
| | | | Immune Pressure within HIV-1 Envelope | |
| | | | Protein Variable region 1 and 2; Immunity 38 | |
| | | | (1), 176-186 (2013), NCBI Accession # | |
| | | | 4HQQ_L (216aa) | |
| HIV21 | Fab Light Chain | Ch59 | Liao et al., vaccine Induction of Antibodies | 27005 |
| | | | against a Structurally Heterogeneous Site of | |
| | | | Immune Pressure within HIV-1 Envelope | |
| | | | Protein Variable region 1 and 2; Immunity 38 | |
| | | | (1), 176-186 (2013), NCBI Accession # | |
| | | | 4HPY_L (215aa) | |
| HIV22 | Fab Light Chain | E51 | Huang C et al., Proc. Natl. Acad. Sci. U.S.A. | 27006 |
| | | | 101 (9), 2706-2711 (2004), NCBI Accession # | |
| | | | 1RZF_L (213aa) | |
| HIV23 | Fab Light Chain | Monoclonal | Bartesaghi, A. et al., Perfusion structure of | 27007 |
| | | Antibody | trimeric HIV-1 envelope glycoprotein | |
| | | Vrc03 | determined by cryo-electron microscopy; Nat. | |
| | | | Struct. Mol. Biol. 20 (12), 1352-1357 (2013), | |
| | | | NCBI Accession # 4CC8_L (209aa) | |
| HIV24 | Fab Light Chain | N26-i1 Fab | NCBI Accession# 4FZE_L (212aa) | 27008 |
| HIV25 | Fab Light Chain | Pgt145 | McLellan, J. S. et al., Structure of HIV-1 gp120 | 27009 |
| | | | V1 V2 domain with broadly neutralizing | |
| | | | antibody PG9; Nature 480 (7377), 336-343 | |
| | | | (2011), NCBI Accession # 3U1S_L (239aa) | |
| HIV26 | Fab Light Chain Of | A32 Fab | NCBI Accession # 3TNM_B (216aa) | 27010 |
| | Human Anti-hiv-1 | | | |
| | Env Antibody A32 | | | |
| HIV27 | Fab Light Chain Of | C11 Fab | NCBI Accession # 4FZ8_L (218aa) | 27011 |
| | Human Anti-hiv-1 | | | |
| | Env Antibody C11 | | | |
| HIV28 | Fab Region Of The | Fab 2558 | Gorny et al., PLoS ONE 6 (12), E27780 (2011), | 27012 |
| | Heavy Chain | | NCBI Accession # 3UJI_H (223aa) | |
| HIV29 | Fab Region Of The | Fab 4025 | Gorny et al., PLoS ONE 6 (12), E27780 (2011), | 27013 |
| | Heavy Chain | | NCBI Accession # 3UJJ_H (230aa) | |
| HIV30 | Fab, Heavy Chain | 3bnc60 | Scheid, J. F., et al,. Science 333 (6049), 1633- | 27014 |
| | | | 1637 (2011), NCBI Accession # 3RPI_A | |
| | | | (229aa) | |
| HIV31 | Fab, Heavy Chain | 48d | Huang C C et al., Proc. Natl. Acad. Sci. U.S.A. | 27015 |
| | | | 101 (9), 2706-2711 (2004), NCBI Accession # | |
| | | | 1R27_H (219aa) | |
| HIV32 | Fab, Heavy Chain | 4e10Fab | Bird et al., Nat. Struct. Mol. Biol. (2014), NCBI | 27016 |
| | | | Accession # 4NGH_H (228aa) | |
| HIV33 | Fab, Heavy Chain | Ch58-ua | Nicely et al. Ebiomedicine 2 (2015), NCBI | 27017 |
| | | | Accession # 4RIS_H (230aa) | |
| HIV34 | Fab, Heavy chain | Mab 2909 | Spurrier, B., et al., Structure 19 (5), 691-699 | 27018 |
| | | | (2011), NCBI Accession # 3Q6F_J (233aa) | |
| HIV35 | Fab, Heavy Chain | Monoclonal | Bartesaghi, A. et al., Perfusion structure of | 27019 |
| | | Antibody | trimeric HIV-1 envelope glycoprotein | |
| | | Vrc03 | determined by cryo-electron microscopy; Nat. | |
| | | | Struct. Mol. Biol. 20 (12), 1352-1357 (2013), | |
| | | | NCBI Accession # 4CC8_I (233aa) | |
| HIV36 | Fab, light chain | 3bnc60 | Scheid, J. F., et al., Science 333 (6049), 1633- | 27020 |
| | | | 1637 (2011), NCBI Accession # 3RPI_B | |
| | | | (206aa) | |
| HIV37 | Fab, Light Chain | 48d | Huang C C et al., Proc. Natl. Acad. Sci. U.S.A. | 27021 |
| | | | 101 (9), 2706-2711. (2004), NCBI Accession # | |
| | | | 1RZ7_L (212aa) | |
| HIV38 | Fab, Light Chain | 4e10Fab | Bird et al., Nat. Struct. Mol. Biol. (2014), NCBI | 27022 |
| | | | Accession # 4NGH_L (215aa) | |
| HIV39 | Fab, Light Chain | Ch58-ua | Nicely et al. Ebiomedicine 2 (2015), NCBI | 27023 |
| | | | Accession # 4RIS_L (216aa) | |
| HIV40 | Fab, light Chain | Mab 2909 | Spurrier, B., et al., Structure 19 (5), 691-699 | 27024 |
| | | | (2011), NCBI Accession # 3Q6F_K (213aa) | |
| HIV41 | Gamma heavy chain | 1443_C16 | U.S. Pat. No. 9,051,362 SEQ ID NO: 12 | 27025 |
| HIV42 | Gamma heavy chain | 1471 M23 | U.S. Pat. No. 9,051,362 SEQ ID NO: 139 | 27026 |
| HIV43 | Gamma heavy chain | 1489_I13 | U.S. Pat. No. 9,051,362 SEQ ID NO: 59 | 27027 |
| HIV44 | Gamma heavy chain | 1503_H05 | U.S. Pat. No. 9,051,362 SEQ ID NO: 53 | 27028 |
| HIV45 | Gamma heavy chain | 1456_A12 | U.S. Pat. No. 9,051,362 SEQ ID NO: 48 | 27029 |
| | variable region | | | |
| HIV46 | Gamma heavy chain | 1456_P20 | U.S. Pat. No. 9,051,362 SEQ ID NO: 33 | 27030 |
| | variable region | | | |
| HIV47 | Gamma heavy chain | 1460_G14 | U.S. Pat. No. 9,051,362 SEQ ID NO: 35 | 27031 |
| | variable region | | | |
| HIV48 | Gamma heavy chain | 1470_M23 | U.S. Pat. No. 9,051,362 SEQ ID NO: 140 | 27032 |
| | variable region | | | |
| HIV49 | Gamma heavy chain | 1480_I08 | U.S. Pat. No. 9,051,362 SEQ ID NO: 31 | 27033 |
| | variable region | | | |
| HIV50 | Gamma heavy chain | 1480_I08 | U.S. Pat. No. 9,051,362 SEQ ID NO: 65 | 27034 |
| | variable region | | | |
| HIV51 | Gamma heavy chain | 1489_I13 | U.S. Pat. No. 9,051,362 SEQ ID NO: 60 | 27035 |
| | variable region | | | |
| HIV52 | Gamma heavy chain | 1495_C14 | U.S. Pat. No. 9,051,362 SEQ ID NO: 37 | 27036 |
| | variable region | | | |
| HIV53 | Gamma heavy chain | 1503_H05 | U.S. Pat. No. 9,051,362 SEQ ID NO: 54 | 27037 |
| | variable region | | | |
| HIV54 | Gamma heavy chain | 1496_C09 | U.S. Pat. No. 9,051,362 SEQ ID NO: 39 | 27038 |
| | variable region | | | |
| HIV55 | Gamma heavy chain | 1456_A12 | U.S. Pat. No. 9,051,362 SEQ ID NO: 47 | 27039 |
| HIV56 | Gamma heavy chain | 1460_G14 | U.S. Pat. No. 9,051,362 SEQ ID NO: 20 | 27040 |
| HIV57 | Gamma heavy chain | 1495_C14 | U.S. Pat. No. 9,051,362 SEQ ID NO: 24 | 27041 |
| HIV58 | Gamma heavy chain | 1496_C09 | U.S. Pat. No. 9,051,362 SEQ ID NO: 28 | 27042 |
| HIV59 | Gamma heavy | 1456_P20 | U.S. Pat. No. 9,051,362 SEQ ID NO: 16 | 27043 |
| HIV60 | Gp41-specific | | US20140348785 SEQ ID NO: 11 | 27044 |
| | antibody, heavy chain | | | |
| HIV61 | Gp41-specific | | US20140348785 SEQ ID NO: 146 | 27045 |
| | antibody, heavy chain | | | |
| | consensus | | | |
| HIV62 | Gp41-specific | | US20140348785 SEQ ID NO: 187 | 27046 |
| | antibody, heavy chain | | | |
| | consensus variable | | | |
| | region | | | |
| HIV63 | Gp41-specific | | US20140348785 SEQ ID NO: 188 | 27047 |
| | antibody, heavy chain | | | |
| | consensus variable | | | |
| | region | | | |
| HIV64 | Gp41-specific | | US20140348785 SEQ ID NO: 153 | 27048 |
| | antibody, heavy chain | | | |
| | variable region | | | |
| HIV65 | Gp41-specific | | US20140348785 SEQ ID NO: 154 | 27049 |
| | antibody, heavy chain | | | |
| | variable region | | | |
| HIV66 | Gp41-specific | | US20140348785 SEQ ID NO: 155 | 27050 |
| | antibody, heavy chain | | | |
| | variable region | | | |
| HIV67 | Gp41-specific | | US20140348785 SEQ ID NO: 156 | 27051 |
| | antibody, heavy chain | | | |
| | variable region | | | |
| HIV68 | Gp41-specific | | US20140348785 SEQ ID NO: 157 | 27052 |
| | antibody, heavy chain | | | |
| | variable region | | | |
| HIV69 | Gp41-specific | | US20140348785 SEQ ID NO: 158 | 27053 |
| | antibody, heavy chain | | | |
| | variable region | | | |
| HIV70 | Gp41-specific | | US20140348785 SEQ ID NO: 159 | 27054 |
| | antibody, heavy chain | | | |
| | variable region | | | |
| HIV71 | Gp41-specific | | US20140348785 SEQ ID NO: 160 | 27055 |
| | antibody, heavy chain | | | |
| | variable region | | | |
| HIV72 | Gp41-specific | | US20140348785 SEQ ID NO: 161 | 27056 |
| | antibody, heavy chain | | | |
| | variable region | | | |
| HIV73 | Gp41-specific | | US20140348785 SEQ ID NO: 162 | 27057 |
| | antibody, heavy chain | | | |
| | variable region | | | |
| HIV74 | Gp41 -specific | | US20140348785 SEQ ID NO: 163 | 27058 |
| | antibody, heavy chain | | | |
| | variable region | | | |
| HIV75 | Gp41-specific | | US20140348785 SEQ ID NO: 189 | 27059 |
| | antibody, heavy chain | | | |
| | variable region | | | |
| HIV76 | Gp41-specific | | US20140348785 SEQ ID NO: 190 | 27060 |
| | antibody, heavy chain | | | |
| | variable region | | | |
| HIV77 | Gp41-specific | | US20140348785 SEQ ID NO: 191 | 27061 |
| | antibody, heavy chain | | | |
| | variable region | | | |
| HIV78 | Gp41-specific | | US20140348785 SEQ ID NO: 192 | 27062 |
| | antibody, heavy chain | | | |
| | variable region | | | |
| HIV79 | Gp41-specific | | US20140348785 SEQ ID NO: 200 | 27063 |
| | antibody, heavy chain | | | |
| | variable region | | | |
| HIV80 | Gp41-specific | | US20140348785 SEQ ID NO: 201 | 27064 |
| | antibody, heavy chain | | | |
| | variable region | | | |
| HIV81 | Gp41-specific | | US20140348785 SEQ ID NO: 202 | 27065 |
| | antibody, heavy chain | | | |
| | variable region | | | |
| HIV82 | Gp41-specific | | US20140348785 SEQ ID NO: 203 | 27066 |
| | antibody, heavy chain | | | |
| | variable region | | | |
| HIV83 | Gp41-specific | | US20140348785 SEQ ID NO: 204 | 27067 |
| | antibody, heavy chain | | | |
| | variable region | | | |
| HIV84 | Gp41-specific | | US20140348785 SEQ ID NO: 205 | 27068 |
| | antibody, heavy chain | | | |
| | variable region | | | |
| HIV85 | Gp41-specific | | US20140348785 SEQ ID NO: 206 | 27069 |
| | antibody, heavy chain | | | |
| | variable region | | | |
| HIV86 | Gp41-specific | | US20140348785 SEQ ID NO: 207 | 27070 |
| | antibody, heavy chain | | | |
| | variable region | | | |
| HIV87 | Gp41-specific | | US20140348785 SEQ ID NO: 208 | 27071 |
| | antibody, heavy chain | | | |
| | variable region | | | |
| HIV88 | Gp41-specific | | US20140348785 SEQ ID NO: 209 | 27072 |
| | antibody, heavy chain | | | |
| | variable region | | | |
| HIV89 | Gp41-specific | | US20140348785 SEQ ID NO: 12 | 27073 |
| | antibody, light chain | | | |
| | variable region | | | |
| HIV90 | Gp41-specific | | US20140348785 SEQ ID NO: 164 | 27074 |
| | antibody, light chain | | | |
| | variable region | | | |
| HIV91 | Gp41-specific | | US20140348785 SEQ ID NO: 165 | 27075 |
| | antibody, light chain | | | |
| | variable region | | | |
| HIV92 | Gp41-specific | | US20140348785 SEQ ID NO: 166 | 27076 |
| | antibody, light chain | | | |
| | variable region | | | |
| HIV93 | Gp41-specific | | US20140348785 SEQ ID NO: 167 | 27077 |
| | antibody, light chain | | | |
| | variable region | | | |
| HIV94 | Gp41-specific | | US20140348785 SEQ ID NO: 168 | 27078 |
| | antibody, light chain | | | |
| | variable region | | | |
| HIV95 | Gp41-specific | | US20140348785 SEQ ID NO: 169 | 27079 |
| | antibody, light chain | | | |
| | variable region | | | |
| HIV96 | Gp41-specific | | US20140348785 SEQ ID NO: 170 | 27080 |
| | antibody, light chain | | | |
| | variable region | | | |
| HIV97 | Gp41-specific | | US20140348785 SEQ ID NO: 171 | 27081 |
| | antibody, light chain | | | |
| | variable region | | | |
| HIV98 | Gp41-specific | | US20140348785 SEQ ID NO: 172 | 27082 |
| | antibody, light chain | | | |
| | variable region | | | |
| HIV99 | Gp41-specific | | US20140348785 SEQ ID NO: 173 | 27083 |
| | antibody, light chain | | | |
| | variable region | | | |
| HIV100 | Gp41-specific | | US20140348785 SEQ ID NO: 174 | 27084 |
| | antibody, light chain | | | |
| | variable region | | | |
| HIV101 | Gp41-specific | | US20140348785 SEQ ID NO: 175 | 27085 |
| | antibody, light chain | | | |
| | variable region | | | |
| HIV102 | Gp41-specific | | US20140348785 SEQ ID NO: 176 | 27086 |
| | antibody, light chain | | | |
| | variable region | | | |
| HIV103 | Gp41-specific | | US20140348785 SEQ ID NO: 177 | 27087 |
| | antibody, light chain | | | |
| | variable region | | | |
| HIV104 | Gp41-specific | | US20140348785 SEQ ID NO: 178 | 27088 |
| | antibody, light chain | | | |
| | variable region | | | |
| HIV105 | Gp41-specific | | US20140348785 SEQ ID NO: 179 | 27089 |
| | antibody, light chain | | | |
| | variable region | | | |
| HIV106 | Gp41-specific | | US20140348785 SEQ ID NO: 180 | 27090 |
| | antibody, light chain | | | |
| | variable region | | | |
| HIV107 | Gp41-specific | | US20140348785 SEQ ID NO: 181 | 27091 |
| | antibody, light chain | | | |
| | variable region | | | |
| HIV108 | Gp41-specific | | US20140348785 SEQ ID NO: 182 | 27092 |
| | antibody, light chain | | | |
| | variable region | | | |
| HIV109 | Gp41-specific | | US20140348785 SEQ ID NO: 183 | 27093 |
| | antibody, light chain | | | |
| | variable region | | | |
| HIV110 | Gp41-specific | | US20140348785 SEQ ID NO: 184 | 27094 |
| | antibody, light chain | | | |
| | variable region | | | |
| HIV111 | Gp41-specific | | US20140348785 SEQ ID NO: 185 | 27095 |
| | antibody, light chain | | | |
| | variable region | | | |
| HIV112 | Gp41-specific | | US20140348785 SEQ ID NO: 186 | 27096 |
| | antibody, light chain | | | |
| | variable region | | | |
| HIV113 | Gp41-specific | | US20140348785 SEQ ID NO: 197 | 27097 |
| | antibody, light chain | | | |
| | variable region | | | |
| HIV114 | Gp41-specific | | US20140348785 SEQ ID NO: 198 | 27098 |
| | antibody, light chain | | | |
| | variable region | | | |
| HIV115 | Gp41-specific | | US20140348785 SEQ ID NO: 199 | 27099 |
| | antibody, light chain | | | |
| | variable region | | | |
| HIV116 | Heavy chain | Vrc06b | Wu, X., et al., Maturation and Diversity of the | 27100 |
| | | | VRC01-Antibody Lineage over 15 Years of | |
| | | | Chronic HIV-1 Infection; Cell 161 (3), 470-485 | |
| | | | (2015), NCBI Accession # 4XNZ_E (234aa) | |
| HIV117 | Heavy Chain | 2424 | Kumar, R., et al., Functional and Structural | 27101 |
| | | | Characterization of Human V3-Specific | |
| | | | Monoclonal Antibody 2424 with Neutralizing | |
| | | | Activity against HIV-1 JRFL; J. Virol. 89 (17), | |
| | | | 9090-9102 (2015), NCBI Accession # | |
| | | | 4XML_H(223aa) | |
| HIV118 | Heavy chain | 5827 | US20140205607 Table S13 | 27102 |
| HIV119 | Heavy chain | 7863 | US20140205607 Table S13 | 27103 |
| HIV120 | Heavy Chain | 8062 | Gustchina, E., PLoS ONE 8 (11), E78187 | 27104 |
| | | | (2013), NCBI Accession # 4KHX_H(245aa) | |
| HIV121 | Heavy chain | 18761 | US20140205607 Table S13 | 27105 |
| HIV122 | Heavy chain | 19891 | US20140205607 Table S13 | 27106 |
| HIV123 | Heavy chain | 22425 | US20140205607 Table S13 | 27107 |
| HIV124 | Heavy chain | 28241 | US20140205607 Table S13 | 27108 |
| HIV125 | Heavy chain | 61272 | US20140205607 Table S13 | 27109 |
| HIV126 | Heavy chain | 61822 | US20140205607 Table S13 | 27110 |
| HIV127 | Heavy chain | 65030 | US20140205607 Table S13 | 27111 |
| HIV128 | Heavy chain | 70085 | US20140205607 Table S13 | 27112 |
| HIV129 | Heavy chain | 70542 | US20140205607 Table S13 | 27113 |
| HIV130 | Heavy chain | 80585 | US20140205607 Table S13 | 27114 |
| HIV131 | Heavy chain | 87722 | US20140205607 Table S13 | 27115 |
| HIV132 | Heavy chain | 96362 | US20140205607 Table S13 | 27116 |
| HIV133 | Heavy chain | 103787 | US20140205607 Table S13 | 27117 |
| HIV134 | Heavy chain | 146940 | US20140205607 Table S13 | 27118 |
| HIV135 | Heavy chain | 153849 | US20140205607 Table S13 | 27119 |
| HIV136 | Heavy chain | 1.00E+09 | US20140348785 SEQ ID NO: 1 | 27120 |
| HIV137 | Heavy chain | 104625_2 | US20140205607 Table S14 | 27121 |
| HIV138 | Heavy chain | 105239_4 | US20140205607 Table S14 | 27122 |
| HIV139 | Heavy chain | 10731_1 | US20140205607 Table S14 | 27123 |
| HIV140 | Heavy Chain | 10e8 | Huang J et al., Nature 491 (7424), 406-412 | 27124 |
| | | (monoclonal) | (2012), NCBI Accession # 4G6F_B (236aa) | |
| HIV141 | Heavy chain | 120119_4 | US20140205607 Table S14 | 27125 |
| HIV142 | Heavy chain | 121325_4 | US20140205607 Table S14 | 27126 |
| HIV143 | Heavy chain | 12467_3 | US20140205607 Table S14 | 27127 |
| HIV144 | Heavy chain | 124918_2 | US20140205607 Table S14 | 27128 |
| HIV145 | Heavy chain | 127586_4 | US20140205607 Table S14 | 27129 |
| HIV146 | Heavy chain | 12A10HC | US20140328862 SEQ ID NO: 147 | 27130 |
| HIV147 | Heavy chain | 12A12HC | US20140328862 SEQ ID NO: 148 | 27131 |
| HIV148 | Heavy chain | 12A13HC | US20140328862 SEQ ID NO: 149 | 27132 |
| HIV149 | Heavy chain | 12A16HC | US20140328862 SEQ ID NO: 150 | 27133 |
| HIV150 | Heavy chain | 12A17HC | US20140328862 SEQ ID NO: 151 | 27134 |
| HIV151 | Heavy chain | 12A1HC | US20140328862 SEQ ID NO: 152 | 27135 |
| HIV152 | Heavy chain | 12A20HC | US20140328862 SEQ ID NO: 153 | 27136 |
| HIV153 | Heavy Chain | 12a21 | NCBI Accession # 4JPW_H (225aa) | 27137 |
| HIV154 | Heavy chain | 12A21HC | US20140328862 SEQ ID NO: 154 | 27138 |
| HIV155 | Heavy chain | I2A22HC | US20140328862 SEQ ID NO: 155 | 27139 |
| HIV156 | Heavy chain | 12A23HC | US20140328862 SEQ ID NO: 156 | 27140 |
| HIV157 | Heavy chain | 12A27HC | US20140328862 SEQ ID NO: 157 | 27141 |
| HIV158 | Heavy chain | 12A2HC | US20140328862 SEQ ID NO: 158 | 27142 |
| HIV159 | Heavy chain | 12A30HC | US20140328862 SEQ ID NO: 159 | 27143 |
| HIV160 | Heavy chain | 12A37HC | US20140328862 SEQ ID NO: 160 | 27144 |
| HIV161 | Heavy chain | 12A46HC | US20140328862 SEQ ID NO: 161 | 27145 |
| HIV162 | Heavy chain | 12A4HC | US20140328862 SEQ ID NO: 162 | 27146 |
| HIV163 | Heavy chain | 12A55HC | US20140328862 SEQ ID NO: 163 | 27147 |
| HIV164 | Heavy chain | 12A56HC | US20140328862 SEQ ID NO: 164 | 27148 |
| HIV165 | Heavy chain | 12A6HC | US20140328862 SEQ ID NO: 165 | 27149 |
| HIV166 | Heavy chain | 12A7HC | US20140328862 SEQ ID NO: 166 | 27150 |
| HIV167 | Heavy chain | 12A9HC | US20140328862 SEQ ID NO: 167 | 27151 |
| HIV168 | Heavy chain | 132797_4 | US20140205607 Table S14 | 27152 |
| HIV169 | Heavy chain | 135083_3 | US20140205607 Table S14 | 27153 |
| HIV170 | Heavy chain | 13826_2 | US20140205607 Table S14 | 27154 |
| HIV171 | Heavy chain | 143251_3 | US20140205607 Table S14 | 27155 |
| HIV172 | Heavy chain | 149590_4 | US20140205607 Table S14 | 27156 |
| HIV173 | Heavy chain | 149768_4 | US20140205607 Table S14 | 27157 |
| HIV174 | Heavy chain | 151901_4 | US20140205607 Table S14 | 27158 |
| HIV175 | Heavy chain | 156858_3 | US20140205607 Table S14 | 27159 |
| HIV176 | Heavy chain | 164202_3 | US20140205607 Table S14 | 27160 |
| HIV177 | Heavy chain | 164922_3 | US20140205607 Table S14 | 27161 |
| HIV178 | Heavy chain | 165478_2 | US20140205607 Table S14 | 27162 |
| HIV179 | Heavy chain | 166726_3 | US20140205607 Table S14 | 27163 |
| HIV180 | Heavy chain | 167612_4 | US20140205607 Table S14 | 27164 |
| HIV181 | Heavy chain | 168509_2 | US20140205607 Table S14 | 27165 |
| HIV182 | Heavy chain | 169094_4 | US20140205607 Table S14 | 27166 |
| HIV183 | Heavy chain | 17720_4 | US20140205607 Table S14 | 27167 |
| HIV184 | Heavy chain | 178037_3 | US20140205607 Table S14 | 27168 |
| HIV185 | Heavy chain | 179400_4 | US20140205607 Table S14 | 27169 |
| HIV186 | Heavy chain | 179500_4 | US20140205607 Table S14 | 27170 |
| HIV187 | Heavy chain | 179888_3 | US20140205607 Table S14 | 27171 |
| HIV188 | Heavy Chain | 17b | Kwong, P. D., et al., structure of an HIV gp120 | 27172 |
| | | | envelope glycoprotein in complex with the CD4 | |
| | | | receptor and a neutralizing human antibody; | |
| | | | Nature 393 (6686), 648-659 (1998), NCBI | |
| | | | Accession# 1G9M_H (229aa) | |
| HIV189 | Heavy chain | 18278_1 | US20140205607 Table S14 | 27173 |
| HIV190 | Heavy chain | 184939_4 | US20140205607 Table S14 | 27174 |
| HIV191 | Heavy chain | 185961_4 | US20140205607 Table S14 | 27175 |
| HIV192 | Heavy chain | 186066_4 | US20140205607 Table S14 | 27176 |
| HIV193 | Heavy chain | 186275_2 | US20140205607 Table S14 | 27177 |
| HIV194 | Heavy chain | 186640_2 | US20140205607 Table S14 | 27178 |
| HIV195 | Heavy chain | 190244_4 | US20140205607 Table S14 | 27179 |
| HIV196 | Heavy chain | 193526_4 | US20140205607 Table S14 | 27180 |
| HIV197 | Heavy chain | 193896_4 | US20140205607 Table S14 | 27181 |
| HIV198 | Heavy chain | 195462_4 | US20140205607 Table S14 | 27182 |
| HIV199 | Heavy chain | 196147_4 | US20140205607 Table S14 | 27183 |
| HIV200 | Heavy chain | 196283_4 | US20140205607 Table S14 | 27184 |
| HIV201 | Heavy Chain | 1b2530 | Zhou T el al., Structural Repertoire of HIV-1- | 27185 |
| | | | Neutralizing Antibodies Targeting the CD4 | |
| | | | Supersite in 14 Donors; Cell 161 (6), 1280- | |
| | | | 1292 (2015), NCBI Accession # 4YFL_H | |
| | | | (227aa) | |
| HIV202 | Heavy chain | 1F7 | U.S. Pat. No. 6,057,421A FIG. 8 | 27186 |
| HIV203 | Heavy chain | 1NC9 | WO2012154312 SEQ ID NO: 2471 | 27187 |
| HIV204 | Heavy Chain | 2.2C | Acharya, P., et al., Structural Definition of an | 27188 |
| | | | Antibody-Dependent Cellular Cytotoxicity | |
| | | | Response Implicated in Reduced Risk for HIV- | |
| | | | 1 Infection; J. Virol. 88 (21), 12895-12906 | |
| | | | (2014), NCBI Accession # 4R4N_H (220aa) | |
| HIV205 | Heavy chain | 24972_4 | US20140205607 Table S14 | 27189 |
| HIV206 | Heavy chain | 28936_1 | US20140205607 Table S14 | 27190 |
| HIV207 | Heavy chain | 2F5 | U.S. Pat. No. 8,637,036B2 SEQ ID NO: 5 | 27191 |
| HIV208 | Heavy chain | 2F5 F100BW | U.S. Pat. No. 8,637,036B2 SEQ ID NO: 7 | 27192 |
| HIV209 | Heavy chain | 2F5 L100AW | U.S. Pat. No. 8,637,036B2 SEQ ID NO: 6 | 27193 |
| HIV210 | Heavy chain | 2F5 | U.S. Pat. No. 8,637,036B2 SEQ ID NO: 9 | 27194 |
| | | L100AW- | | |
| | | V100DW | | |
| HIV211 | Heavy chain | 2F5 | U.S. Pat. No. 8,637,036B2 SEQ ID NO: 8 | 27195 |
| | | V100DW | | |
| HIV212 | Heavy chain | 30263_2 | US20140205607 Table S14 | 27196 |
| HIV213 | Heavy chain | 3040HC | WO2015117008 SEQ ID NO: 14 | 27197 |
| HIV214 | Heavy chain | 3044HC | WO20S5117008 SEQ ID NO: 17 | 27198 |
| HIV215 | Heavy chain | 31458_3 | US20140205607 Table S14 | 27199 |
| HIV216 | Heavy chain | 3430HC | WO2015117008 SEQ ID NO: 15 | 27200 |
| HIV217 | Heavy chain | 3484HC | WO2015117008 SEQ ID NO: 16 | 27201 |
| HIV218 | Heavy chain | 3630HC | WO2015117008 SEQ ID NO: 18 | 27202 |
| HIV219 | Heavy chain | 3A124HC | US20140328862 SEQ ID NO: 261 | 27203 |
| HIV220 | Heavy chain | 3A125HC | US20140328862 SEQ ID NO: 262 | 27204 |
| HIV221 | Heavy chain | 3A140HC | US20140328862 SEQ ID NO: 263 | 27205 |
| HIV222 | Heavy chain | 3A144HC | US20140328862 SEQ ID NO: 264 | 27206 |
| HIV223 | Heavy chain | 3A160HC | US20140328862 SEQ ID NO: 265 | 27207 |
| HIV224 | Heavy chain | 3A18HC | US20140328862 SEQ ID NO: 266 | 27208 |
| HIV225 | Heavy chain | 3A204HC | US20140328862 SEQ ID NO: 267 | 27209 |
| HIV226 | Heavy chain | 3A228HC | US20140328862 SEQ ID NO: 268 | 27210 |
| HIV227 | Heavy chain | 3A233HC | US20140328862 SEQ ID NO: 269 | 27211 |
| HIV228 | Heavy chain | 3A244HC | US20140328862 SEQ ID NO: 270 | 27212 |
| HIV229 | Heavy chain | 3A255HC | US20140328862 SEQ ID NO: 271 | 27213 |
| HIV230 | Heavy chain | 3A296HC | US20140328862 SEQ ID NO: 272 | 27214 |
| HIV231 | Heavy chain | 3A334HC | US20140328862 SEQ ID NO: 273 | 27215 |
| HIV232 | Heavy chain | 3A366HC | US20140328862 SEQ ID NO: 274 | 27216 |
| HIV233 | Heavy chain | 3A381HC | US20140328862 SEQ ID NO: 275 | 27217 |
| HIV234 | Heavy chain | 3A384HC | US20140328862 SEQ ID NO: 276 | 27218 |
| HIV235 | Heavy chain | 3A419HC | US20140328862 SEQ ID NO: 277 | 27219 |
| HIV236 | Heavy chain | 3a426hc | US20140328862 SEQ ID NO: 343 | 27220 |
| HIV237 | Heavy chain | 3A461HC | US20140328862 SEQ ID NO: 278 | 27221 |
| HIV238 | Heavy chain | 3A474HC | US20140328862 SEQ ID NO: 279 | 27222 |
| HIV239 | Heavy chain | 3a515hc | US20140328862 SEQ ID NO: 344 | 27223 |
| HIV240 | Heavy chain | 3A518HC | US20140328862 SEQ ID NO: 280 | 27224 |
| HIV241 | Heavy chain | 3A539HC | US20140328862 SEQ ID NO: 281 | 27225 |
| HIV242 | Heavy chain | 3A576HC | US20140328862 SEQ ID NO: 282 | 27226 |
| HIV243 | Heavy chain | 3A613HC | US20140328862 SEQ ID NO: 283 | 27227 |
| HIV244 | Heavy chain | 3A64HC | US20140328862 SEQ ID NO: 284 | 27228 |
| HIV245 | Heavy chain | 3A650HC | US20140328862 SEQ ID NO: 285 | 27229 |
| HIV246 | Heavy chain | 3A67HC | US20140328862 SEQ ID NO: 286 | 27230 |
| HIV247 | Heavy chain | 3A779HC | US20140328862 SEQ ID NO: 287 | 27231 |
| HIV248 | Heavy chain | 3A816HC | US20140328862 SEQ ID NO: 288 | 27232 |
| HIV249 | Heavy chain | 3A869HC | US20140328862 SEQ ID NO: 289 | 27233 |
| HIV250 | Heavy chain | 3A93HC | US20140328862 SEQ ID NO: 290 | 27234 |
| HIV251 | Heavy chain | 3A966HC | US20140328862 SEQ ID NO: 291 | 27235 |
| HIV252 | Heavy chain | 3A978HC | US20140328862 SEQ ID NO: 292 | 27236 |
| HIV253 | Heavy chain | 3ANC32HC | US20140328862 SEQ ID NO: 346 | 27237 |
| HIV254 | Heavy chain | 3ANC3HC | US20140328862 SEQ ID NO: 293 | 27238 |
| HIV255 | Heavy chain | 3ANC3HC | US20140328862 SEQ ID NO: 347 | 27239 |
| HIV256 | Heavy chain | 3ANC41HC | US20140328862 SEQ ID NO: 348 | 27240 |
| HIV257 | Heavy chain | 3ANC42HC | US20140328862 SEQ ID NO: 294 | 27241 |
| HIV258 | Heavy chain | 3ANC42HC | US20140328862 SEQ ID NO: 349 | 27242 |
| HIV259 | Heavy chain | 3ANC66HC | US20140328862 SEQ ID NO: 295 | 27243 |
| HIV260 | Heavy chain | 3ANC66HC | US20140328862 SEQ ID NO: 350 | 27244 |
| HIV261 | Heavy chain | 3ANC70HC | US20140328862 SEQ ID NO: 351 | 27245 |
| HIV262 | Heavy chain | 3ANC75HC | US20140328862 SEQ ID NO: 352 | 27246 |
| HIV263 | Heavy chain | 3ANC79HC | US20140328862 SEQ ID NO: 296 | 27247 |
| HIV264 | Heavy chain | 3ANC79HC | US20140328862 SEQ ID NO: 353 | 27248 |
| HIV265 | Heavy chain | 3ANC87HC | US20140328862 SEQ ID NO: 354 | 27249 |
| HIV266 | Heavy chain | 3ANC8HC | US20140328862 SEQ ID NO: 355 | 27250 |
| HIV267 | Heavy chain | 3ANC96HC | US20140328862 SEQ ID NO: 356 | 27251 |
| HIV268 | Heavy chain | 3B106HC | US20140328862 SEQ ID NO: 357 | 27252 |
| HIV269 | Heavy chain | 3B10HC | US20140328862 SEQ ID NO: 297 | 27253 |
| HIV270 | Heavy chain | 3B120HC | US20140328862 SEQ ID NO: 298 | 27254 |
| HIV271 | Heavy chain | 3B126HC | US20140328862 SEQ ID NO: 299 | 27255 |
| HIV272 | Heavy chain | 3B129HC | US20140328862 SEQ ID NO: 300 | 27256 |
| HIV273 | Heavy chain | 3B142HC | US20140328862 SEQ ID NO: 301 | 27257 |
| HIV274 | Heavy chain | 3B154HC | US20140328862 SEQ ID NO: 302 | 27258 |
| HIV275 | Heavy chain | 3B165HC | US20140328862 SEQ ID NO: 303 | 27259 |
| HIV276 | Heavy chain | 3B16HC | US20140328862 SEQ ID NO: 358 | 27260 |
| HIV277 | Heavy chain | 3B171HC | US20140328862 SEQ ID NO: 304 | 27261 |
| HIV278 | Heavy chain | 3B17HC | US20140328862 SEQ ID NO: 305 | 27262 |
| HIV279 | Heavy chain | 3B180HC | US20140328862 SEQ ID NO: 359 | 27263 |
| HIV280 | Heavy chain | 3B183HC | US20140328862 SEQ ID NO: 360 | 27264 |
| HIV281 | Heavy chain | 3B186HC | US20140328862 SEQ ID NO: 306 | 27265 |
| HIV282 | Heavy chain | 3B191HC | US20140328862 SEQ ID NO: 361 | 27266 |
| HIV283 | Heavy chain | 3B193HC | US20140328862 SEQ ID NO: 307 | 27267 |
| HIV284 | Heavy chain | 3B21HC | US20140328862 SEQ ID NO: 362 | 27268 |
| HIV285 | Heavy chain | 3B22HC | US20140328862 SEQ ID NO: 308 | 27269 |
| HIV286 | Heavy chain | 3B27HC | US20140328862 SEQ ID NO: 309 | 27270 |
| HIV287 | Heavy chain | 3B29HC | US20140328862 SEQ ID NO: 310 | 27271 |
| HIV288 | Heavy chain | 3B2HC | US20140328862 SEQ ID NO: 311 | 27272 |
| HIV289 | Heavy chain | 3B31HC | US20140328862 SEQ ID NO: 312 | 27273 |
| HIV290 | Heavy chain | 3B33HC | US20140328862 SEQ ID NO: 313 | 27274 |
| HIV291 | Heavy chain | 3B40HC | US20140328862 SEQ ID NO: 314 | 27275 |
| HIV292 | Heavy chain | 3B41HC | US20140328862 SEQ ID NO: 315 | 27276 |
| HIV293 | Heavy chain | 3B44HC | US20140328862 SEQ ID NO: 316 | 27277 |
| HIV294 | Heavy chain | 3B45HC | US20140328862 SEQ ID NO: 317 | 27278 |
| HIV295 | Heavy chain | 3b46HC | US20140328862 SEQ ID NO: 345 | 27279 |
| HIV296 | Heavy chain | 3B48HC | US20140328862 SEQ ID NO: 318 | 27280 |
| HIV297 | Heavy chain | 3B50HC | US20140328862 SEQ ID NO: 319 | 27281 |
| HIV298 | Heavy chain | 3B51HC | US20140328862 SEQ ID NO: 320 | 27282 |
| HIV299 | Heavy chain | 3B56HC | US20140328862 SEQ ID NO: 321 | 27283 |
| HIV300 | Heavy chain | 3B57HC | US20140328862 SEQ ID NO: 322 | 27284 |
| HIV301 | Heavy chain | 3B5HC | US20140328862 SEQ ID NO: 323 | 27285 |
| HIV302 | Heavy chain | 3B61HC | US20140328862 SEQ ID NO: 324 | 27286 |
| HIV303 | Heavy chain | 3B6HC | US20140328862 SEQ ID NO: 325 | 27287 |
| HIV304 | Heavy chain | 3B77HC | US20140328862 SEQ ID NO: 326 | 27288 |
| HIV305 | Heavy chain | 3B79HC | US20140328862 SEQ ID NO: 327 | 27289 |
| HIV306 | Heavy chain | 3B84HC | US20140328862 SEQ ID NO: 328 | 27290 |
| HIV307 | Heavy chain | 3B86HC | US20140328862 SEQ ID NO: 329 | 27291 |
| HIV308 | Heavy chain | 3B8HC | US20140328862 SEQ ID NO: 330 | 27292 |
| HIV309 | Heavy chain | 3B93HC | US20140328862 SEQ ID NO: 331 | 27293 |
| HIV310 | Heavy chain | 3BBM60 | US20140328862 SEQ ID NO: 363 | 27294 |
| HIV311 | Heavy chain | 3BBM60 | US20140328862 SEQ ID NO: 364 | 27295 |
| HIV312 | Heavy chain | 3BBM60 | US20140328862 SEQ ID NO: 365 | 27296 |
| HIV313 | Heavy chain | 3BBM60 | US20140328862 SEQ ID NO: 366 | 27297 |
| HIV314 | Heavy chain | 3BBM60 | US20140328862 SEQ ID NO: 367 | 27298 |
| HIV315 | Heavy chain | 3BBM60 | US20140328862 SEQ ID NO: 368 | 27299 |
| HIV316 | Heavy chain | 3BBM60 | US20140328862 SEQ ID NO: 369 | 27300 |
| HIV317 | Heavy chain | 3BBM60 | US20140328862 SEQ ID NO: 370 | 27301 |
| HIV318 | Heavy chain | 3BBM60 | US20140328862 SEQ ID NO: 371 | 27302 |
| HIV319 | Heavy chain | 3BBM60 | US20140328862 SEQ ID NO: 372 | 27303 |
| HIV320 | Heavy chain | 3BBM60 | US20140328862 SEQ ID NO: 373 | 27304 |
| HIV321 | Heavy chain | 3BBM60 | US20140328862 SEQ ID NO: 374 | 27305 |
| HIV322 | Heavy chain | 3BBM60 | US20140328862 SEQ ID NO: 375 | 27306 |
| HIV323 | Heavy chain | 3BBM60 | US20140328862 SEQ ID NO: 376 | 27307 |
| HIV324 | Heavy chain | 3BBM60 | US20140328862 SEQ ID NO: 377 | 27308 |
| HIV325 | Heavy chain | 3BNC101HC | US20140328862 SEQ ID NO: 332 | 27309 |
| HIV326 | Heavy chain | 3BNC101HC | US20140328862 SEQ ID NO: 378 | 27310 |
| HIV327 | Heavy chain | 3BNC102HC | US20140328862 SEQ ID NO: 379 | 27311 |
| HIV328 | Heavy chain | 3BNC104HC | US20140328862 SEQ ID NO: 380 | 27312 |
| HIV329 | Heavy chain | 3BNC105HC | US20140328862 SEQ ID NO: 381 | 27313 |
| HIV330 | Heavy chain | 3BNC106HC | US20140328862 SEQ ID NO: 382 | 27314 |
| HIV331 | Heavy chain | 3BNC107HC | US20140328862 SEQ ID NO: 383 | 27315 |
| HIV332 | Heavy chain | 3BNC108HC | US20140328862 SEQ ID NO: 384 | 27316 |
| HIV333 | Heavy chain | 3BNC10HC | US20140328862 SEQ ID NO: 385 | 27317 |
| HIV334 | Heavy chain | 3BNC114HC | US20140328862 SEQ ID NO: 386 | 27318 |
| HIV335 | Heavy Chain | 3bnc117 | Zhou T et al., Immunity 39 (2), 245-258 (2013), | 27319 |
| | | | NCBI Accession # 4LSV_H (226aa) | |
| HIV336 | Heavy chain | 3BNC117HC | US20140328862 SEQ ID NO: 387 | 27320 |
| HIV337 | Heavy chain | 3BNC124HC | US20140328862 SEQ ID NO: 333 | 27321 |
| HIV338 | Heavy chain | 3BNC126HC | US20140328862 SEQ ID NO: 388 | 27322 |
| HIV339 | Heavy chain | 3BNC127HC | US20140328862 SEQ ID NO: 389 | 27323 |
| HIV340 | Heavy chain | 3BNC130HC | US20140328862 SEQ ID NO: 334 | 27324 |
| HIV341 | Heavy chain | 3BNC134HC | US20140328862 SEQ ID NO: 390 | 27325 |
| HIV342 | Heavy chain | 3BNC140HC | US20140328862 SEQ ID NO: 391 | 27326 |
| HIV343 | Heavy chain | 3BNC141HC | US20140328862 SEQ ID NO: 392 | 27327 |
| HIV344 | Heavy chain | 3BNC142HC | US20140328862 SEQ ID NO: 393 | 27328 |
| HIV345 | Heavy chain | 3BNC148HC | US20140328862 SEQ ID NO: 394 | 27329 |
| HIV346 | Heavy chain | 3BNC149HC | US20140328862 SEQ ID NO: 335 | 27330 |
| HIV347 | Heavy chain | 3BNC149HC | US20140328862 SEQ ID NO: 395 | 27331 |
| HIV348 | Heavy chain | 3BNC151HC | US20140328862 SEQ ID NO: 396 | 27332 |
| HIV349 | Heavy chain | 3BNC153HC | US20140328862 SEQ ID NO: 397 | 27333 |
| HIV350 | Heavy chain | 3BNC156HC | US20140328862 SEQ ID NO: 398 | 27334 |
| HIV351 | Heavy chain | 3BNC158HC | US20140328862 SEQ ID NO: 399 | 27335 |
| HIV352 | Heavy chain | 3BNC159HC | US20140328862 SEQ ID NO: 400 | 27336 |
| HIV353 | Heavy chain | 3BNC15HC | US20140328862 SEQ ID NO: 401 | 27337 |
| HIV354 | Heavy chain | 3BNC173HC | US20140328862 SEQ ID NO: 402 | 27338 |
| HIV355 | Heavy chain | 3BNC175HC | US20140328862 SEQ ID NO: 403 | 27339 |
| HIV356 | Heavy chain | 3BNC176HC | US20140328862 SEQ ID NO: 404 | 27340 |
| HIV357 | Heavy chain | 3BNC177HC | US20140328862 SEQ ID NO: 336 | 27341 |
| HIV358 | Heavy chain | 3BNC17HC | US20140328862 SEQ ID NO: 337 | 27342 |
| HIV359 | Heavy chain | 3BNC181HC | US20140328862 SEQ ID NO: 405 | 27343 |
| HIV360 | Heavy chain | 3BNC186HC | US20140328862 SEQ ID NO: 406 | 27344 |
| HIV361 | Heavy chain | 3BNC18HC | US20140328862 SEQ ID NO: 407 | 27345 |
| HIV362 | Heavy chain | 3BNC193HC | US20140328862 SEQ ID NO: 408 | 27346 |
| HIV363 | Heavy chain | 3BNC196HC | US20140328862 SEQ ID NO: 409 | 27347 |
| HIV364 | Heavy chain | 3BNC20HC | US20140328862 SEQ ID NO: 410 | 27348 |
| HIV365 | Heavy chain | 3BNC29HC | US20140328862 SEQ ID NO: 411 | 27349 |
| HIV366 | Heavy chain | 3BNC31HC | US20140328862 SEQ ID NO: 412 | 27350 |
| HIV367 | Heavy chain | 3BNC33HC | US20140328862 SEQ ID NO: 413 | 27351 |
| HIV368 | Heavy chain | 3BNC42HC | US20140328862 SEQ ID NO: 414 | 27352 |
| HIV369 | Heavy chain | 3BNC44HC | US20140328862 SEQ ID NO: 415 | 27353 |
| HIV370 | Heavy chain | 3BNC45HC | US20140328862 SEQ ID NO: 416 | 27354 |
| HIV371 | Heavy chain | 3BNC48HC | US20140328862 SEQ ID NO: 338 | 27355 |
| HIV372 | Heavy chain | 3BNC53HC | US20140328862 SEQ ID NO: 417 | 27356 |
| HIV373 | Heavy chain | 3BNC54HC | US20140328862 SEQ ID NO: 418 | 27357 |
| HIV374 | Heavy chain | 3BNC55HC | US20140328862 SEQ ID NO: 419 | 27358 |
| HIV375 | Heavy chain | 3BNC58HC | US20140328862 SEQ ID NO: 339 | 27359 |
| HIV376 | Heavy chain | 3BNC59HC | US20140328862 SEQ ID NO: 420 | 27360 |
| HIV377 | Heavy chain | 3BNC60HC | US20140328862 SEQ ID NO: 421 | 27361 |
| HIV378 | Heavy chain | 3BNC62HC | US20140328862 SEQ ID NO: 422 | 27362 |
| HIV379 | Heavy chain | 3BNC64HC | US20140328862 SEQ ID NO: 423 | 27363 |
| HIV380 | Heavy chain | 3BNC65HC | US20140328862 SEQ ID NO: 424 | 27364 |
| HIV381 | Heavy chain | 3BNC66HC | US20140328862 SEQ ID NO: 425 | 27365 |
| HIV382 | Heavy chain | 3BNC6HC | US20140328862 SEQ ID NO: 426 | 27366 |
| HIV383 | Heavy chain | 3BNC72HC | US20140328862 SEQ ID NO: 427 | 27367 |
| HIV384 | Heavy chain | 3BNC75HC | US20140328862 SEQ ID NO: 428 | 27368 |
| HIV385 | Heavy chain | 3BNC78HC | US20140328862 SEQ ID NO: 340 | 27369 |
| HIV386 | Heavy chain | 3BNC79HC | US20140328862 SEQ ID NO: 429 | 27370 |
| HIV387 | Heavy chain | 3BNC81HC | US20140328862 SEQ ID NO: 430 | 27371 |
| HIV388 | Heavy chain | 3BNC82HC | US20140328862 SEQ ID NO: 341 | 27372 |
| HIV389 | Heavy chain | 3BNC84HC | US20140328862 SEQ ID NO: 431 | 27373 |
| HIV390 | Heavy chain | 3BNC86HC | US20140328862 SEQ ID NO: 432 | 27374 |
| HIV391 | Heavy chain | 3BNC87HC | US20140328862 SEQ ID NO: 433 | 27375 |
| HIV392 | Heavy chain | 3BNC89HC | US20140328862 SEQ ID NO: 434 | 27376 |
| HIV393 | Heavy chain | 3BNC8HC | US20140328862 SEQ ID NO: 342 | 27377 |
| HIV394 | Heavy chain | 3BNC91HC | US20140328862 SEQ ID NO: 435 | 27378 |
| HIV395 | Heavy chain | 3BNC92HC | US20140328862 SEQ ID NO: 436 | 27379 |
| HIV396 | Heavy chain | 3BNC94HC | US20140328862 SEQ ID NO: 437 | 27380 |
| HIV397 | Heavy chain | 3BNC95HC | US20140328862 SEQ ID NO: 438 | 27381 |
| HIV398 | Heavy Chain | 412d | Huang et al., Science 317 (5846), 1930-1934 | 27382 |
| | | | (2007), NCBI Accession # 2QAD_H (231aa) | |
| HIV399 | Heavy chain | 43243_3 | US20140205607 Table S14 | 27383 |
| HIV400 | Heavy chain | 43359_2 | US20140205607 Table S14 | 27384 |
| HIV401 | Heavy chain | 43555_1 | US20140205607 Table S14 | 27385 |
| HIV402 | Heavy chain | 43567_2 | US20140205607 Table S14 | 27386 |
| HIV403 | Heavy Chain | 44-vrc13.01 | Zhou T et al., Structural Repertoire of HIV-1- | 27387 |
| | | | Neutralizing Antibodies Targeting the CD4 | |
| | | | Supersite in 14 Donors; Cell 161 (6), 1280- | |
| | | | 1292 (2015), NCBI Accession # | |
| | | | 4YDJ_A(238aa) | |
| HIV404 | Heavy chain | 45-46m2 | Diskin, R., et al., Restricting HIV-1 pathways | 27388 |
| | | | for escape using rationally designed anti-HIV-1 | |
| | | | antibodies; J. Exp. Med. 210 (6), 1235-1249 | |
| | | | (2013), NCBI Accession # 4JKP_H (229aa) | |
| HIV405 | Heavy chain | 46260_1 | US20140205607 Table S14 | 27389 |
| HIV406 | Heavy chain | 47890_1 | US20140205607 Table S14 | 27390 |
| HIV407 | Heavy Chain | 4e10 Fv | Finton, K. A., et al., PLoS Pathol. 9 (9), | 27391 |
| | | | E1003639 (2013), NCBI Accession # 4LLV_A | |
| | | | (129aa) | |
| HIV408 | Heavy chain | 53821_1 | US20140205607 Table S14 | 27392 |
| HIV409 | Heavy chain | 57729_2 | US20140205607 Table S14 | 27393 |
| HIV410 | Heavy chain | 61048_1 | US20140205607 Table S14 | 27394 |
| HIV411 | Heavy chain | 69713_1 | US20140205607 Table S14 | 27395 |
| HIV412 | Heavy chain | 70679_1 | US20140205607 Table S14 | 27396 |
| HIV413 | Heavy chain | 71632_2 | US20140205607 Table S14 | 27397 |
| HIV414 | Heavy chain | 74400_3 | US20140205607 Table S14 | 27398 |
| HIV415 | Heavy chain | 74511_1 | US20140205607 Table S14 | 27399 |
| HIV416 | Heavy chain | 76927_2 | US20140205607 Table S14 | 27400 |
| HIV417 | Heavy Chain | 7b2 | Santra, S., et al., PLoS Pathol. 11 (8), | 27401 |
| | | | E1005042 (2015), NCBI Accession # 4YDV_H | |
| | | | (252aa) | |
| HIV418 | Heavy chain | 7H6 | US20140348785 SEQ ID NO: 3 | 27402 |
| HIV419 | Heavy chain | 7N16 | US20140348785 SEQ ID NO: 5 | 27403 |
| HIV420 | Heavy chain | 8460_4 | US20140205607 Table S14 | 27404 |
| HIV421 | Heavy chain | 86277_2 | US20140205607 Table S14 | 27405 |
| HIV422 | Heavy chain | 86343_1 | US20140205607 Table S14 | 27406 |
| HIV423 | Heavy chain | 86984_2 | US20140205607 Table S14 | 27407 |
| HIV424 | Heavy chain | 89680_4 | US20140205607 Table S14 | 27408 |
| HIV425 | Heavy chain | 8A253HC | US20140328862 SEQ ID NO: 5 | 27409 |
| HIV426 | Heavy chain | 8A275HC | US20140328862 SEQ ID NO: 6 | 27410 |
| HIV427 | Heavy chain | 8ABM11 | US20140328862 SEQ ID NO: 7 | 27411 |
| HIV428 | Heavy chain | 8ABM12 | US20140328862 SEQ ID NO: 8 | 27412 |
| HIV429 | Heavy chain | 8ABM13 | US20140328862 SEQ ID NO: 9 | 27413 |
| HIV430 | Heavy chain | 8ABM14 | US20140328862 SEQ ID NO: 10 | 27414 |
| HIV431 | Heavy chain | 8ABM20 | US20140328862 SEQ ID NO: 11 | 27415 |
| HIV432 | Heavy chain | 8ABM24 | US20140328862 SEQ ID NO: 12 | 27416 |
| HIV433 | Heavy chain | 8ABM26 | US20140328862 SEQ ID NO: 13 | 27417 |
| HIV434 | Heavy chain | 8ABM27 | US20140328862 SEQ ID NO: 14 | 27418 |
| HIV435 | Heavy chain | 8ANC103HC | US20140328862 SEQ ID NO: 36 | 27419 |
| HIV436 | Heavy chain | 8ANC105HC | US20140328862 SEQ ID NO: 15 | 27420 |
| HIV437 | Heavy chain | 8ANC106HC | US20140328862 SEQ ID NO: 37 | 27421 |
| HIV438 | Heavy chain | 8ANC107HC | US20140328862 SEQ ID NO: 38 | 27422 |
| HIV439 | Heavy chain | 8ANC108HC | US20140328862 SEQ ID NO: 39 | 27423 |
| HIV440 | Heavy chain | 8ANC109HC | US20140328862 SEQ ID NO: 40 | 27424 |
| HIV441 | Heavy chain | 8ANC10HC | US20140328862 SEQ ID NO: 41 | 27425 |
| HIV442 | Heavy chain | 8ANC111HC | US20140328862 SEQ ID NO: 42 | 27426 |
| HIV443 | Heavy chain | 8ANC112HC | US20140328862 SEQ ID NO: 43 | 27427 |
| HIV444 | Heavy chain | 8ANC113HC | US20140328862 SEQ ID NO: 44 | 27428 |
| HIV445 | Heavy chain | 8ANC114HC | US20140328862 SEQ ID NO: 45 | 27429 |
| HIV446 | Heavy chain | 8ANC115HC | US20140328862 SEQ ID NO: 46 | 27430 |
| HIV447 | Heavy chain | 8ANC116HC | US20140328862 SEQ ID NO: 16 | 27431 |
| HIV448 | Heavy chain | 8ANC117HC | US20140328862 SEQ ID NO: 47 | 27432 |
| HIV449 | Heavy chain | 8ANC11HC | US20140328862 SEQ ID NO: 48 | 27433 |
| HIV450 | Heavy chain | 8ANC121HC | US20140328862 SEQ ID NO: 49 | 27434 |
| HIV451 | Heavy chain | 8ANC126HC | US20140328862 SEQ ID NO: 50 | 27435 |
| HIV452 | Heavy chain | 8ANC127HC | US20140328862 SEQ ID NO: 17 | 27436 |
| HIV453 | Heavy chain | 8ANC130HC | US20140328862 SEQ ID NO: 51 | 27437 |
| HIV454 | Heavy Chain | 8anc131 | Zhou T et al., Structural Repertoire of HIV-1- | 27438 |
| | | | Neutralizing Antibodies Targeting the CD4 | |
| | | | Supersite in 14 Donors; Cell 161 (6), 1280- | |
| | | | 1292 (2015), NCBI Accession # 4RWY_H | |
| | | | (227aa) | |
| HIV455 | Heavy chain | 8ANC131HC | US20140328862 SEQ ID NO: 18 | 27439 |
| HIV456 | Heavy chain | 8ANC132HC | US20140328862 SEQ ID NO: 52 | 27440 |
| HIV457 | Heavy chain | 8ANC133HC | US20140328862 SEQ ID NO: 53 | 27441 |
| HIV458 | Heavy Chain | 8anc134 | Zhou T et al., Structural Repertoire of HIV-1- | 27442 |
| | | | Neutralizing Antibodies Targeting the CD4 | |
| | | | Supersite in 14 Donors; Cell 161 (6), 1280- | |
| | | | 1292 (2015), NCBI Accession # 4RX4_H | |
| | | | (229aa) | |
| HIV459 | Heavy chain | 8ANC134HC | US20140328862 SEQ ID NO: 19 | 27443 |
| HIV460 | Heavy chain | 8ANC136HC | US20140328862 SEQ ID NO: 54 | 27444 |
| HIV461 | Heavy chain | 8ANC137HC | US20140328862 SEQ ID NO: 55 | 27445 |
| HIV462 | Heavy chain | 8ANC139HC | US20140328862 SEQ ID NO: 56 | 27446 |
| HIV463 | Heavy chain | 8ANC13HC | US20140328862 SEQ ID NO: 20 | 27447 |
| HIV464 | Heavy chain | 8ANC140HC | US20140328862 SEQ ID NO: 57 | 27448 |
| HIV465 | Heavy chain | 8ANC142HC | US20140328862 SEQ ID NO: 58 | 27449 |
| HIV466 | Heavy chain | 8ANC143HC | US20140328862 SEQ ID NO: 59 | 27450 |
| HIV467 | Heavy chain | 8ANC144HC | US20140328862 SEQ ID NO: 60 | 27451 |
| HIV468 | Heavy chain | 8ANC145HC | US20140328862 SEQ ID NO: 61 | 27452 |
| HIV469 | Heavy chain | 8ANC146HC | US20140328862 SEQ ID NO: 62 | 27453 |
| HIV470 | Heavy chain | 8ANC147HC | US20140328862 SEQ ID NO: 63 | 27454 |
| HIV471 | Heavy chain | 8ANC148HC | US20140328862 SEQ ID NO: 64 | 27455 |
| HIV472 | Heavy chain | 8ANC149HC | US20140328862 SEQ ID NO: 65 | 27456 |
| HIV473 | Heavy chain | 8ANC14HC | US20140328862 SEQ ID NO: 66 | 27457 |
| HIV474 | Heavy chain | 8ANC150HC | US20140328862 SEQ ID NO: 67 | 27458 |
| HIV475 | Heavy chain | 8ANC151HC | US20140328862 SEQ ID NO: 68 | 27459 |
| HIV476 | Heavy chain | 8ANC153HC | US20140328862 SEQ ID NO: 69 | 27460 |
| HIV477 | Heavy chain | 8ANC154HC | US20140328862 SEQ ID NO: 70 | 27461 |
| HIV478 | Heavy chain | 8ANC155HC | US20140328862 SEQ ID NO: 71 | 27462 |
| HIV479 | Heavy chain | 8ANC156HC | US20140328862 SEQ ID NO: 72 | 27463 |
| HIV480 | Heavy chain | 8ANC157HC | US20140328862 SEQ ID NO: 73 | 27464 |
| HIV481 | Heavy chain | 8ANC158HC | US20140328862 SEQ ID NO: 74 | 27465 |
| HIV482 | Heavy chain | 8ANC160HC | US20140328862 SEQ ID NO: 75 | 27466 |
| HIV483 | Heavy chain | 8ANC161HC | US20140328862 SEQ ID NO: 76 | 27467 |
| HIV484 | Heavy chain | 8ANC162HC | US20140328862 SEQ ID NO: 77 | 27468 |
| HIV485 | Heavy chain | 8ANC163HC | US20140328862 SEQ ID NO: 78 | 27469 |
| HIV486 | Heavy chain | 8ANC164HC | US20140328862 SEQ ID NO: 79 | 27470 |
| HIV487 | Heavy chain | 8ANC165HC | US20140328862 SEQ ID NO: 80 | 27471 |
| HIV488 | Heavy chain | 8ANC166HC | US20140328862 SEQ ID NO: 81 | 27472 |
| HIV489 | Heavy chain | 8ANC168HC | US20140328862 SEQ ID NO: 82 | 27473 |
| HIV490 | Heavy chain | 8ANC169HC | US20140328862 SEQ ID NO: 83 | 27474 |
| HIV491 | Heavy chain | 8ANC16HC | US20140328862 SEQ ID NO: 84 | 27475 |
| HIV492 | Heavy chain | 8ANC171HC | US20140328862 SEQ ID NO: 21 | 27476 |
| HIV493 | Heavy chain | 8ANC173HC | US20140328862 SEQ ID NO: 85 | 27477 |
| HIV494 | Heavy chain | 8ANC174HC | US20140328862 SEQ ID NO: 86 | 27478 |
| HIV495 | Heavy chain | 8ANC175HC | US20140328862 SEQ ID NO: 87 | 27479 |
| HIV496 | Heavy chain | 8ANC176HC | US20140328862 SEQ ID NO: 88 | 27480 |
| HIV497 | Heavy chain | 8ANC177HC | US20140328862 SEQ ID NO: 89 | 27481 |
| HIV498 | Heavy chain | 8ANC178HC | US20140328862 SEQ ID NO: 90 | 27482 |
| HIV499 | Heavy chain | 8ANC179HC | US20140328862 SEQ ID NO: 91 | 27483 |
| HIV500 | Heavy chain | 8ANC17HC | US20140328862 SEQ ID NO: 92 | 27484 |
| HIV501 | Heavy chain | 8ANC18 | US20140328862 SEQ ID NO: 22 | 27485 |
| HIV502 | Heavy chain | 8ANC180HC | US20140328862 SEQ ID NO: 93 | 27486 |
| HIV503 | Heavy chain | 8ANC181HC | US20140328862 SEQ ID NO: 94 | 27487 |
| HIV504 | Heavy chain | 8ANC182HC | US20140328862 SEQ ID NO: 23 | 27488 |
| HIV505 | Heavy chain | 8ANC184HC | US20140328862 SEQ ID NO: 95 | 27489 |
| HIV506 | Heavy chain | 8ANC185HC | US20140328862 SEQ ID NO: 96 | 27490 |
| HIV507 | Heavy chain | 8ANC186HC | US20140328862 SEQ ID NO: 97 | 27491 |
| HIV508 | Heavy chain | 8ANC187HC | US20140328862 SEQ ID NO: 98 | 27492 |
| HIV509 | Heavy chain | 8ANC188HC | US20140328862 SEQ ID NO: 99 | 27493 |
| HIV510 | Heavy chain | 8ANC191HC | US20140328862 SEQ ID NO: 100 | 27494 |
| HIV511 | Heavy chain | 8ANC192HC | US20140328862 SEQ ID NO: 24 | 27495 |
| HIV512 | Heavy chain | 8ANC193HC | US20140328862 SEQ ID NO: 101 | 27496 |
| HIV513 | Heavy chain | 8ANC194HC | US20140328862 SEQ ID NO: 102 | 27497 |
| HIV514 | Heavy chain | 8ANC195HC | US20140328862 SEQ ID NO: 103 | 27498 |
| HIV515 | Heavy chain | 8ANC196HC | US20140328862 SEQ ID NO: 104 | 27499 |
| HIV516 | Heavy chain | 8ANC20HC | US20140328862 SEQ ID NO: 105 | 27500 |
| HIV517 | Heavy chain | 8ANC21HC | US20140328862 SEQ ID NO: 106 | 27501 |
| HIV518 | Heavy chain | 8ANC22HC | US20140328862 SEQ ID NO: 25 | 27502 |
| HIV519 | Heavy chain | 8ANC24HC | US20140328862 SEQ ID NO: 107 | 27503 |
| HIV520 | Heavy chain | 8ANC25HC | US20140328862 SEQ ID NO: 108 | 27504 |
| HIV521 | Heavy chain | 8ANC26HC | US20140328862 SEQ ID NO: 26 | 27505 |
| HIV522 | Heavy chain | 8ANC27HC | US20140328862 SEQ ID NO: 109 | 27506 |
| HIV523 | Heavy chain | 8ANC2HC | US20140328862 SEQ ID NO: 27 | 27507 |
| HIV524 | Heavy chain | 8ANC30HC | US20140328862 SEQ ID NO: 28 | 27508 |
| HIV525 | Heavy chain | 8ANC31HC | US20140328862 SEQ ID NO: 110 | 27509 |
| HIV526 | Heavy chain | 8ANC33HC | US20140328862 SEQ ID NO: 111 | 27510 |
| HIV527 | Heavy chain | 8ANC34HC | US20140328862 SEQ ID NO: 112 | 27511 |
| HIV528 | Heavy chain | 8ANC36HC | US20140328862 SEQ ID NO: 113 | 27512 |
| HIV529 | Heavy chain | 8ANC37HC | US20140328862 SEQ ID NO: 29 | 27513 |
| HIV530 | Heavy chain | 8ANC38HC | US20140328862 SEQ ID NO: 114 | 27514 |
| HIV531 | Heavy chain | 8ANC39HC | US20140328862 SEQ ID NO: 115 | 27515 |
| HIV532 | Heavy chain | 8ANC3HC | US20140328862 SEQ ID NO: 116 | 27516 |
| HIV533 | Heavy chain | 8ANC40HC | US20140328862 SEQ ID NO: 30 | 27517 |
| HIV534 | Heavy chain | 8ANC41HC | US20140328862 SEQ ID NO: 31 | 27518 |
| HIV535 | Heavy chain | 8ANC43HC | US20140328862 SEQ ID NO: 117 | 27519 |
| HIV536 | Heavy chain | 8ANC45HC | US20140328862 SEQ ID NO: 32 | 27520 |
| HIV537 | Heavy chain | 8ANC46HC | US20140328862 SEQ ID NO: 118 | 27521 |
| HIV538 | Heavy chain | 8ANC48HC | US20140328862 SEQ ID NO: 119 | 27522 |
| HIV539 | Heavy chain | 8ANC49HC | US20140328862 SEQ ID NO: 120 | 27523 |
| HIV540 | Heavy chain | 8ANC50HC | US20140328862 SEQ ID NO: 33 | 27524 |
| HIV541 | Heavy chain | 8ANC51HC | US20140328862 SEQ ID NO: 121 | 27525 |
| HIV542 | Heavy chain | 8ANC53HC | US20140328862 SEQ ID NO: 34 | 27526 |
| HIV543 | Heavy chain | 8ANC57HC | US20140328862 SEQ ID NO: 122 | 27527 |
| HIV544 | Heavy chain | 8ANC58HC | US20140328862 SEQ ID NO: 123 | 27528 |
| HIV545 | Heavy chain | 8ANC5HC | US20140328862 SEQ ID NO: 124 | 27529 |
| HIV546 | Heavy chain | 8ANC60HC | US20140328862 SEQ ID NO: 125 | 27530 |
| HIV547 | Heavy chain | 8ANC63HC | US20140328862 SEQ ID NO: 126 | 27531 |
| HIV548 | Heavy chain | 8ANC65HC | US20140328862 SEQ ID NO: 127 | 27532 |
| HIV549 | Heavy chain | 8ANC67HC | US20140328862 SEQ ID NO: 128 | 27533 |
| HIV550 | Heavy chain | 8ANC69HC | US20140328862 SEQ ID NO: 129 | 27534 |
| HIV551 | Heavy chain | 8ANC6HC | US20140328862 SEQ ID NO: 130 | 27535 |
| HIV552 | Heavy chain | 8ANC70HC | US20140328862 SEQ ID NO: 131 | 27536 |
| HIV553 | Heavy chain | 8ANC71HC | US20140328862 SEQ ID NO: 132 | 27537 |
| HIV554 | Heavy chain | 8ANC72HC | US20140328862 SEQ ID NO: 133 | 27538 |
| HIV555 | Heavy chain | 8ANC74HC | US20140328862 SEQ ID NO: 134 | 27539 |
| HIV556 | Heavy chain | 8ANC75HC | US20140328862 SEQ ID NO: 135 | 27540 |
| HIV557 | Heavy chain | 8ANC76HC | US20140328862 SEQ ID NO: 136 | 27541 |
| HIV558 | Heavy chain | 8ANC78HC | US20140328862 SEQ ID NO: 137 | 27542 |
| HIV559 | Heavy chain | 8ANC79HC | US20140328862 SEQ ID NO: 138 | 27543 |
| HIV560 | Heavy chain | 8ANC7HC | US20140328862 SEQ ID NO: 139 | 27544 |
| HIV561 | Heavy chain | 8ANC80HC | US20140328862 SEQ ID NO: 140 | 27545 |
| HIV562 | Heavy chain | 8ANC82HC | US20140328862 SEQ ID NO: 141 | 27546 |
| HIV563 | Heavy chain | 8ANC83HC | US20140328862 SEQ ID NO: 142 | 27547 |
| HIV564 | Heavy chain | 8ANC88HC | US20140328862 SEQ ID NO: 35 | 27548 |
| HIV565 | Heavy chain | 8ANC91HC | US20140328862 SEQ ID NO: 143 | 27549 |
| HIV566 | Heavy chain | 8ANC92HC | US20140328862 SEQ ID NO: 144 | 27550 |
| HIV567 | Heavy chain | 8ANC93HC | US20140328862 SEQ ID NO: 145 | 27551 |
| HIV568 | Heavy chain | 8ANC9HC | US20140328862 SEQ ID NO: 146 | 27552 |
| HIV569 | Heavy chain | 94565_1 | US20140205607 Table S14 | 27553 |
| HIV570 | Heavy chain | 95589_2 | US20140205607 Table S14 | 27554 |
| HIV571 | Heavy chain | 96298_1 | US20140205607 Table S14 | 27555 |
| HIV572 | Heavy chain | 9815_2 | US20140205607 Table S14 | 27556 |
| HIV573 | Heavy chain | 99473_3 | US20140205607 Table S14 | 27557 |
| HIV574 | Heavy chain | 99989_1 | US20140205607 Table S14 | 27558 |
| HIV575 | Heavy chain | Antibody | US20140328862 SEQ ID NO: 439 | 27559 |
| HIV576 | Heavy chain | Anti-HcG | Fotinou C. et al “Structure of an Fab fragment | 27560 |
| | | | against a C-terminal peptide of hCG at 2.0 A | |
| | | | resolution” J. Biol. Chem. 273 (35), 22515- | |
| | | | 22518 (1998); NCBI Accession # 1SBS_H | |
| HIV577 | Heavy Chain | B12 | Zhou T et al., Structural definition of a | 27561 |
| | | | conserved neutralization epitope on HIV-1 | |
| | | | gp120; Nature 445 (7129), 732-737 (2007), | |
| | | | NCBI Accession # 2NY7_H (230aa) | |
| HIV578 | Heavy Chain | C38-vrc16.01 | Zhou T et al., Structural Repertoire of HIV-1- | 27562 |
| | | | Neutralizing Antibodies Targeting the CD4 | |
| | | | Supersite in 14 Donors; Cell 161 (6), 1280- | |
| | | | 1292 (2015), NCBI Accession # 4YDK_H | |
| | | | (234aa) | |
| HIV579 | Heavy Chain | C38-vrc18.02 | Zhou T et al., Structural Repertoire of HIV-1- | 27563 |
| | | | Neutralizing Antibodies Targeting the CD4 | |
| | | | Supersite in 14 Donors; Cell 161 (6), 1280- | |
| | | | 1292 (2015), NCBI Accession # 4YDL_H | |
| | | | (226aa) | |
| HIV580 | Heavy chain | CAP256- | WO2015128846 SEQ ID NO: 13 | 27564 |
| | | VRC26.01 | | |
| HIV581 | Heavy chain | CAP256- | WO2015128846 SEQ ID NO: 17 | 27565 |
| | | VRC26.02 | | |
| HIV582 | Heavy chain | CAP256- | WO2015128846 SEQ ID NO: 21 | 27566 |
| | | VRC26.03 | | |
| HIV583 | Heavy chain | CAP256- | WO2015128846 SEQ ID NO: 25 | 27567 |
| | | VRC26.04 | | |
| HIV584 | Heavy chain | CAP256- | WO2015128846 SEQ ID NO: 29 | 27568 |
| | | VRC26.05 | | |
| HIV585 | Heavy chain | CAP256- | WO2015128846 SEQ ID NO: 33 | 27569 |
| | | VRC26.06 | | |
| HIV586 | Heavy chain | CAP256- | WO2015128846 SEQ ID NO: 37 | 27570 |
| | | VRC26.07 | | |
| HIV587 | Heavy chain | CAP256- | WO2015128846 SEQ ID NO: 41 | 27571 |
| | | VRC26.08 | | |
| HIV588 | Heavy chain | CAP256- | WO2015128846 SEQ ID NO: 45 | 27572 |
| | | VRC26.09 | | |
| HIV589 | Heavy chain | CAP256- | WO2015128846 SEQ ID NO: 49 | 27573 |
| | | VRC26.10 | | |
| HIV590 | Heavy chain | CAP256- | WO2015128846 SEQ ID NO: 53 | 27574 |
| | | VRC26.11 | | |
| HIV591 | Heavy chain | CAP256- | WO2015128846 SEQ ID NO: 57 | 27575 |
| | | VRC26.12 | | |
| HIV592 | Heavy chain | CAP256- | WO2015128846 SEQ ID NO: 170 | 27576 |
| | | VRC26.25 | | |
| HIV593 | Heavy chain | CAP256- | WO2015128846 SEQ ID NO: 178 | 27577 |
| | | VRC26.26 | | |
| HIV594 | Heavy chain | CAP256- | WO2015128846 SEQ ID NO: 186 | 27578 |
| | | VRC26.27 | | |
| HIV595 | Heavy chain | CAP256- | WO2015128846 SEQ ID NO: 5 | 27579 |
| | | VRC26-I1 | | |
| HIV596 | Heavy chain | CAP256- | WO2015128846 SEQ ID NO: 9 | 27580 |
| | | VRC26-I2. | | |
| HIV597 | Heavy chain | CAP256- | WO2015128846 SEQ ID NO: 1 | 27581 |
| | | VRC26- | | |
| | | UCA. | | |
| HIV598 | Heavy chain | construct | WO2015013390 SEQ ID NO: 3 | 27582 |
| | | #2816, #2859 | | |
| HIV599 | Heavy chain | construct | WO2015013390 SEQ ID NO: 4 | 27583 |
| | | #2817 | | |
| HIV600 | Heavy chain | construct | WO2015013390 SEQ ID NO: 8 | 27584 |
| | | #2858, #2860 | | |
| HIV601 | Heavy Chain | Fab 2219 | Stanfield, R. L., et al., J. Virol. 80 (12), 6093- | 27585 |
| | | | 6105 (2006), NCBI Accession # 2B0S_H | |
| | | | (226aa) | |
| HIV602 | Heavy Chain | Fab 2g12 | Doores. K. J., et al., J. Virol. 84 (20), 10690- | 27586 |
| | | | 10699 (2010), NCBI Accession # 3OAU_H | |
| | | | (225aa) | |
| HIV603 | Heavy Chain | Fab 2g12 | Stanfield, R. L. et al., Crystal structure of the | 27587 |
| | | | HIV neutralizing antibody 2G12 in complex | |
| | | | with a bacterial oligosaccharide analog of | |
| | | | mammalian oligomannose; Glycobiology 25 | |
| | | | (4), 412-419 (2015), NCBI Accession # | |
| | | | 4RBP_H (224aa) | |
| HIV604 | Heavy Chain | Fab F425- | Bell et al., J. Mol. Biol. 375 (4), 969-978 | 27588 |
| | | b4e8 | (2008), NCBI Accession # 2QSC_H (222aa) | |
| HIV605 | Heavy chain | fusion protein | US20080038280 SEQ ID NO: 5 | 27589 |
| | | of A32 and | | |
| | | m9 | | |
| HIV606 | Heavy chain | g20 | WO2015117008 SEQ ID NO: 4 | 27590 |
| HIV607 | Heavy chain | g22 | WO2015117008 SEQ ID NO: 7 | 27591 |
| HIV608 | Heavy chain | g23 | WO2015117008 SEQ ID NO: 2 | 27592 |
| HIV609 | Heavy chain | g3 | WO2015117008 SEQ ID NO: 13 | 27593 |
| HIV610 | Heavy chain | g4 | WO2015117008 SEQ ID NO: 9 | 27594 |
| HIV611 | Heavy chain | g44 | WO2015117008 SEQ ID NO: 11 | 27595 |
| HIV612 | Heavy chain | g46 | WO2015117008 SEQ ID NO: 10 | 27596 |
| HIV613 | Heavy chain | G4D | US20130195881 SEQ ID NO: 9 | 27597 |
| HIV614 | Heavy chain | G4H | US20130195881 SEQ ID NO: 8 | 27598 |
| HIV615 | Heavy chain | g50 | WO2015117008 SEQ ID NO: 12 | 27599 |
| HIV616 | Heavy chain | g52 | WO2015117008 SEQ ID NO: 1 | 27600 |
| HIV617 | Heavy chain | g59 | WO2015117008 SEQ ID NO: 5 | 27601 |
| HIV618 | Heavy chain | g62 | WO2015117008 SEQ ID NO: 6 | 27602 |
| HIV619 | Heavy chain | g8 | WO2015117008 SEQ ID NO: 3 | 27603 |
| HIV620 | Heavy chain | gl5 | WO2015117008 SEQ ID NO: 8 | 27604 |
| HIV621 | Heavy chain | gVRC- | WO2013090644 SEQ ID NO: 45 | 27605 |
| | | H5(d74)/VR | | |
| | | C-PG04LC | | |
| HIV622 | Heavy chain | gVRCOH12(D74)/ | WO2013090644 SEQ ID NO: 46 | 27606 |
| | | VRC- | | |
| | | PG04LC | | |
| HIV623 | Heavy Chain | I2 (unbound) | Fera, D. et al., Affinity maturation in an HIV | 27607 |
| | | From Ch103 | broadly neutralizing B-cell lineage through | |
| | | Lineage | reorientation of variable domains; Proc. Natl. | |
| | | | Acad. Sci. U.S.A. 111 (28), 10275-10280 | |
| | | | (2014), NCBI Accession # 4QHN_A (232aa) | |
| HIV624 | Heavy chain | IGHV3- | US20140348785 SEQ ID NO: 7 | 27608 |
| | | 15*05 | | |
| HIV625 | Heavy chain | LSSB2055HC | US20140328862 SEQ ID NO: 229 | 27609 |
| HIV626 | Heavy chain | LSSB2066HC | US20140328862 SEQ ID NO: 230 | 27610 |
| HIV627 | Heavy chain | LSSB2068HC | US20140328862 SEQ ID NO: 231 | 27611 |
| HIV628 | Heavy chain | LSSB2080HC | US20140328862 SEQ ID NO: 232 | 27612 |
| HIV629 | Heavy chain | LSSB2133HC | US20140328862 SEQ ID NO: 233 | 27613 |
| HIV630 | Heavy chain | LSSB2182HC | US20140328862 SEQ ID NO: 234 | 27614 |
| HIV631 | Heavy chain | LSSB218HC | US20140328862 SEQ ID NO: 235 | 27615 |
| HIV632 | Heavy chain | LSSB2277HC | US20140328862 SEQ ID NO: 236 | 27616 |
| HIV633 | Heavy chain | LSSB2288HC | US20140328862 SEQ ID NO: 237 | 27617 |
| HIV634 | Heavy chain | LSSB2339HC | US20140328862 SEQ ID NO: 168 | 27618 |
| HIV635 | Heavy chain | LSSB2351HC | US20140328862 SEQ ID NO: 169 | 27619 |
| HIV636 | Heavy chain | LSSB2361HC | US20140328862 SEQ ID NO: 170 | 27620 |
| HIV637 | Heavy chain | LSSB2364HC | US20140328862 SEQ ID NO: 171 | 27621 |
| HIV638 | Heavy chain | LSSB2367HC | US20140328862 SEQ ID NO: 172 | 27622 |
| HIV639 | Heavy chain | LSSB2416HC | US20140328862 SEQ ID NO: 173 | 27623 |
| HIV640 | Heavy chain | LSSB2434HC | US20140328862 SEQ ID NO: 174 | 27624 |
| HIV641 | Heavy chain | LSSB2483HC | US20140328862 SEQ ID NO: 175 | 27625 |
| HIV642 | Heavy chain | LSSB2490HC | US20140328862 SEQ ID NO: 176 | 27626 |
| HIV643 | Heavy chain | LSSB2503HC | US20140328862 SEQ ID NO: 177 | 27627 |
| HIV644 | Heavy chain | LSSB2525HC | US20140328862 SEQ ID NO: 178 | 27628 |
| HIV645 | Heavy chain | LSSB2530HC | US20140328862 SEQ ID NO: 179 | 27629 |
| HIV646 | Heavy chain | LSSB2538HC | US20140328862 SEQ ID NO: 180 | 27630 |
| HIV647 | Heavy chain | LSSB2554HC | US20140328862 SEQ ID NO: 181 | 27631 |
| HIV648 | Heavy chain | LSSB2573HC | US20140328862 SEQ ID NO: 182 | 27632 |
| HIV649 | Heavy chain | LSSB2578HC | US20140328862 SEQ ID NO: 183 | 27633 |
| HIV650 | Heavy chain | LSSB2586HC | US20140328862 SEQ ID NO: 184 | 27634 |
| HIV651 | Heavy chain | LSSB2609HC | US20140328862 SEQ ID NO: 185 | 27635 |
| HIV652 | Heavy chain | LSSB2612HC | US20140328862 SEQ ID NO: 186 | 27636 |
| HIV653 | Heavy chain | LSSB2630HC | US20140328862 SEQ ID NO: 187 | 27637 |
| HIV654 | Heavy chain | LSSB2640HC | US20S40328862 SEQ ID NO: 188 | 27638 |
| HIV655 | Heavy chain | LSSB2644HC | US20140328862 SEQ ID NO: 189 | 27639 |
| HIV656 | Heavy chain | LSSB2665HC | US20S40328862 SEQ ID NO: 190 | 27640 |
| HIV657 | Heavy chain | LSSB2666HC | US20140328862 SEQ ID NO: 191 | 27641 |
| HIV658 | Heavy chain | LSSB2669HC | US20S40328862 SEQ ID NO: 192 | 27642 |
| HIV659 | Heavy chain | LSSB2680HC | US20140328862 SEQ ID NO: 193 | 27643 |
| HIV660 | Heavy chain | LSSB2683HC | US20S40328862 SEQ ID NO: 194 | 27644 |
| HIV661 | Heavy chain | LSSB331HC | US20140328862 SEQ ID NO: 238 | 27645 |
| HIV662 | Heavy chain | LSSB344HC | US20140328862 SEQ ID NO: 195 | 27646 |
| HIV663 | Heavy chain | LSSNEC101HC | US20140328862 SEQ ID NO: 239 | 27647 |
| HIV664 | Heavy chain | LSSNEC106HC | US20140328862 SEQ ID NO: 240 | 27648 |
| HIV665 | Heavy chain | LSSNEC107HC | US20140328862 SEQ ID NO: 196 | 27649 |
| HIV666 | Heavy chain | LSSNEC108HC | US20140328862 SEQ ID NO: 197 | 27650 |
| HIV667 | Heavy chain | LSSNEC109HC | US20140328862 SEQ ID NO: 198 | 27651 |
| HIV668 | Heavy chain | LSSNEC110HC | US20140328862 SEQ ID NO: 199 | 27652 |
| HIV669 | Heavy chain | LSSNEC112HC | US20140328862 SEQ ID NO: 241 | 27653 |
| HIV670 | Heavy chain | LSSNEC115HC | US20140328862 SEQ ID NO: 242 | 27654 |
| HIV671 | Heavy chain | LSSNEC116HC | US20140328862 SEQ ID NO: 200 | 27655 |
| HIV672 | Heavy chain | LSSNEC117HC | US20140328862 SEQ ID NO: 201 | 27656 |
| HIV673 | Heavy chain | LSSNEC118HC | US20140328862 SEQ ID NO: 202 | 27657 |
| HIV674 | Heavy chain | LSSNEC11HC | US20140328862 SEQ ID NO: 203 | 27658 |
| HIV675 | Heavy chain | LSSNEC122HC | US20140328862 SEQ ID NO: 204 | 27659 |
| HIV676 | Heavy chain | LSSNEC123HC | US20140328862 SEQ ID NO: 205 | 27660 |
| HIV677 | Heavy chain | LSSNEC124HC | US20140328862 SEQ ID NO: 243 | 27661 |
| HIV678 | Heavy chain | LSSNEC125HC | US20140328862 SEQ ID NO: 244 | 27662 |
| HIV679 | Heavy chain | LSSNEC126HC | US20140328862 SEQ ID NO: 245 | 27663 |
| HIV680 | Heavy chain | LSSNEC127HC | US20140328862 SEQ ID NO: 206 | 27664 |
| HIV681 | Heavy chain | LSSNEC14HC | US20140328862 SEQ ID NO: 246 | 27665 |
| HIV682 | Heavy chain | LSSNEC16HC | US20140328862 SEQ ID NO: 247 | 27666 |
| HIV683 | Heavy chain | LSSNEC18HC | US20140328862 SEQ ID NO: 207 | 27667 |
| HIV684 | Heavy chain | LSSNEC21HC | US20140328862 SEQ ID NO: 248 | 27668 |
| HIV685 | Heavy chain | LSSNEC24HC | US20140328862 SEQ ID NO: 208 | 27669 |
| HIV686 | Heavy chain | LSSNEC29HC | US20140328862 SEQ ID NO: 209 | 27670 |
| HIV687 | Heavy chain | LSSNEC2HC | US20140328862 SEQ ID NO: 210 | 27671 |
| HIV688 | Heavy chain | LSSNEC30HC | US20140328862 SEQ ID NO: 249 | 27672 |
| HIV689 | Heavy chain | LSSNEC33HC | US20140328862 SEQ ID NO: 211 | 27673 |
| HIV690 | Heavy chain | LSSNEC34HC | US20140328862 SEQ ID NO: 212 | 27674 |
| HIV691 | Heavy chain | LSSNEC3HC | US20140328862 SEQ ID NO: 213 | 27675 |
| HIV692 | Heavy chain | LSSNEC46HC | US20140328862 SEQ ID NO: 214 | 27676 |
| HIV693 | Heavy chain | LSSNEC48HC | US20140328862 SEQ ID NO: 215 | 27677 |
| HIV694 | Heavy chain | LSSNEC49HC | US20140328862 SEQ ID NO: 250 | 27678 |
| HIV695 | Heavy chain | LSSNEC52HC | US20140328862 SEQ ID NO: 216 | 27679 |
| HIV696 | Heavy chain | LSSNEC54HC | US20140328862 SEQ ID NO: 251 | 27680 |
| HIV697 | Heavy chain | LSSNEC55HC | US20140328862 SEQ ID NO: 252 | 27681 |
| HIV698 | Heavy chain | LSSNEC56HC | US20140328862 SEQ ID NO: 217 | 27682 |
| HIV699 | Heavy chain | LSSNEC57HC | US20140328862 SEQ ID NO: 253 | 27683 |
| HIV700 | Heavy chain | LSSNEC5HC | US20140328862 SEQ ID NO: 254 | 27684 |
| HIV701 | Heavy chain | LSSNEC60HC | US20140328862 SEQ ID NO: 218 | 27685 |
| HIV702 | Heavy chain | LSSNEC66HC | US20140328862 SEQ ID NO: 219 | 27686 |
| HIV703 | Heavy chain | LSSNEC67HC | US20140328862 SEQ ID NO: 255 | 27687 |
| HIV704 | Heavy chain | LSSNEC70HC | US20140328862 SEQ ID NO: 220 | 27688 |
| HIV705 | Heavy chain | LSSNEC72HC | US20140328862 SEQ ID NO: 221 | 27689 |
| HIV706 | Heavy chain | LSSNEC74HC | US20140328862 SEQ ID NO: 256 | 27690 |
| HIV707 | Heavy chain | LSSNEC77HC | US20140328862 SEQ ID NO: 257 | 27691 |
| HIV708 | Heavy chain | LSSNEC7HC | US20140328862 SEQ ID NO: 222 | 27692 |
| HIV709 | Heavy chain | LSSNEC82HC | US20140328862 SEQ ID NO: 223 | 27693 |
| HIV710 | Heavy chain | LSSNEC85HC | US20140328862 SEQ ID NO: 258 | 27694 |
| HIV711 | Heavy chain | LSSNEC89HC | US20140328862 SEQ ID NO: 224 | 27695 |
| HIV712 | Heavy chain | LSSNEC8HC | US20140328862 SEQ ID NO: 225 | 27696 |
| HIV713 | Heavy chain | LSSNEC91HC | US20140328862 SEQ ID NO: 259 | 27697 |
| HIV714 | Heavy chain | LSSNEC92HC | US20140328862 SEQ ID NO: 260 | 27698 |
| HIV715 | Heavy chain | LSSNEC94HC | US20140328862 SEQ ID NO: 226 | 27699 |
| HIV716 | Heavy chain | LSSNEC95HC | US20140328862 SEQ ID NO: 227 | 27700 |
| HIV717 | Heavy chain | LSSNEC9HC | US20140328862 SEQ ID NO: 228 | 27701 |
| HIV718 | Heavy chain | m12_Fd-aa | U.S. Pat. No. 7,803,913B2 SEQ ID NO: 3 | 27702 |
| HIV719 | Heavy chain | m14-Fd-aa | U.S. Pat. No. 7,803,913B2 SEQ ID NO: 1 | 27703 |
| HIV720 | Heavy chain | m16-Fd-aa | U.S. Pat. No. 7,803,913B2 SEQ ID NO: 4 | 27704 |
| HIV721 | Heavy chain | m18 Fd-aa | U.S. Pat. No. 7,803,913B2 SEQ ID NO: 2 | 27705 |
| HIV722 | Heavy Chain | M66 | Ofek, G., et al., Structural Basis for HIV-1 | 27706 |
| | | | Neutralization by 2F5-Like Antibodies m66 | |
| | | | and m66.6; J. Virol. 88 (5), 2426-2441 (2014), | |
| | | | NCBI Accession # 4NRY_L (220aa) | |
| HIV723 | Heavy Chain | M66.6 | Ofek, G., et al., Structural Basis for HIV-1 | 27707 |
| | | | Neutralization by 2F5-Like Antibodies m66 | |
| | | | and m66.6; J. Virol. 88 (5), 2426-2441 (2014), | |
| | | | NCBI Accession # 4NRZ_H (234aa) | |
| HIV724 | Heavy Chain | Mab 2158 | Spurrier, B., et al., Functional Implications of | 27708 |
| | | | the Binding Mode of a Human Conformation- | |
| | | | Dependent V2 Monoclonal Antibody against | |
| | | | HIV; J. Virol, 88 (8), 4100-4112 (2014), NCBI | |
| | | | Accession # 4OAW_D (236aa) | |
| HIV725 | Heavy chain | MV1 | US20130195881 SEQ ID NO: 10 | 27709 |
| HIV726 | Heavy Chain | Pg16 Fab | Pancera, M., et al., Nat. Struct. Mol. Biol. 20 | 27710 |
| | | | (7), 804-813 (2013), NCBI Accession # | |
| | | | 4DQO_H (246aa) | |
| HIV727 | Heavy Chain | Pg9 | Willis, J. R., et al., J. Clin. Invest. 125 (6), 2523- | 27711 |
| | | | 2531 (2015), NCBI Accession # | |
| | | | 4YAQ_H(248aa) | |
| HIV728 | Heavy Chain | Pgt121-Gl | Mouquet H et al., Complex-type N-glycan | 27712 |
| | | Fab | recognition by potent broadly neutralizing HIV | |
| | | | antibodies; Proc Natl Acad Sci USA. 2012 | |
| | | | Nov. 20; 109(47): E3268-77, NCBI Accession # | |
| | | | 4FQQ_B (244aa) | |
| HIV729 | Heavy Chain | Pgt122 | Julien, J. P., et al., PLoS Pathol. 9 (5), | 27713 |
| | | | E1003342 (2013), NCBI Accession # 4JY5_H | |
| | | | (235aa) | |
| HIV730 | Heavy Chain | Pgt123 | Julien, J. P., et al., PLoS Pathol. 9 (5), | 27714 |
| | | | E1003342 (2013), NCBI Accession # 4JY6_B | |
| | | | (235aa) | |
| HIV731 | Heavy Chain | Pgt124 | Garces, F., et al., Structural Evolution of | 27715 |
| | | | Glycan Recognition by a Family of Potent HIV | |
| | | | Antibodies; Cell 159 (1), 69-79 (2014), NCBI | |
| | | | Accession # 4R26_H (236aa) | |
| HIV732 | Heavy Chain | Pgt130 | Doores, K. J., et al., J. Virol. 89 (2), 1105-1118 | 27716 |
| | | | (2015), NCBI Accession # 4RNR_A (233aa) | |
| HIV733 | Heavy Chain | Pgt135 | Grover et al., Science 343 (6171), 656-661 | 27717 |
| | | | (2014), NCBI Accession # 4NZR_H (234aa) | |
| HIV734 | Heavy chain | S19 | US20110059015 SEQ ID NO: 6 | 27718 |
| HIV735 | Heavy chain | S20 | US20110059015 SEQ ID NO: 8 | 27719 |
| HIV736 | Heavy chain | S8 | US20110059015 SEQ ID NO: 4 | 27720 |
| HIV737 | Heavy Chain | Vrc- Pg04 | Wu, X., et al., Focused evolution of HIV-1 | 27721 |
| | | | neutralizing antibodies revealed by structures | |
| | | | and deep sequencing; Science 333 (6049), | |
| | | | 1593-1602 (2011)”, NCBI Accession # | |
| | | | 3SE9_H (228aa) | |
| HIV738 | Heavy chain | VRC01 | U.S. Pat. No. 8,637,036B2 SEQ ID NO: 1 | 27722 |
| HIV739 | Heavy chain | VRC01HC/VRCO3LC | WO2013090644 SEQ ID NO: 2 | 27723 |
| HIV740 | Heavy chain | VRC02 | U.S. Pat. No. 8,637,036B2 SEQ ID NO: 3 | 27724 |
| HIV741 | Heavy chain | VRC03 | U.S. Pat. No. 8,637,036B2 SEQ ID NO: 27 | 27725 |
| HIV742 | Heavy chain | VRC03HC- | WO2013090644 SEQ ID NO: 32 | 27726 |
| | | VRC01LC | | |
| HIV743 | Heavy chain | VRC07 | US20140322163 SEQ ID NO: 258 | 27727 |
| | | G54H, S58N | | |
| HIV744 | Heavy chain | VRC07 I37V, | US20140322163 SEQ ID NO: 260 | 27728 |
| | | G54H, S58N, | | |
| | | T93A | | |
| HIV745 | Heavy chain | VRC07 I37V, | US20140322163 SEQ ID NO: 259 | 27729 |
| | | G54H, T93A | | |
| HIV746 | Heavy Chain | Vrc08c | Wu, X., et al., Maturation and Diversity of the | 27730 |
| | | | VRC01-Antibody Lineage over 15 Years of | |
| | | | Chronic HIV-1 Infection; Cell 161 (3), 470-485 | |
| | | | (2015), NCBI Accession # 4XNY_H (235aa) | |
| HIV747 | Heavy Chain | Vrc23 | Georgiev, I. S., et al., Delineating antibody | 27731 |
| | | | recognition in polyclonal sera from patterns of | |
| | | | HIV-1 isolate neutralization; Science 340 | |
| | | | (6133), 751-756 (2013), NCBI Accession # | |
| | | | 4J6R_H (224aa) | |
| HIV748 | Heavy chain | VRC-CH30 | WO2013090644 SEQ ID NO: 22 | 27732 |
| HIV749 | Heavy Chain | Vrc-ch31 | Zhou T et al., Immunity 39 (2), 245-258 (2013), | 27733 |
| | | | NCBI Accession # 4LSP_H (236aa) | |
| HIV750 | Heavy chain | VRC-CH32 | Wu X. et al, “Focused evolution of HIV-1 | 27734 |
| | | | neutralizing antibodies revealed by structures | |
| | | | and deep sequencing” Science 333 (6049), | |
| | | | 1593-1602 (2011), NCBI Accession # | |
| | | | AEM62724 | |
| HIV751 | Heavy chain | VRC-CH33 | WO2013090644 SEQ ID NO: 28 | 27735 |
| HIV752 | Heavy chain | VRC-CH34 | WO2013090644 SEQ ID NO: 30 | 27736 |
| HIV753 | Heavy chain | VRCO7 | US20140322163 SEQ ID NO: 33 | 27737 |
| | | G54H | | |
| HIV754 | Heavy chain | VRC-PG04 | Wu X. et al, “Focused evolution of HIV-1 | 27738 |
| | | | neutralizing antibodies revealed by structures | |
| | | | and deep sequencing” Science 333 (6049), | |
| | | | 1593-1602 (2011), NCBI Accession # | |
| | | | AEM62752 | |
| HIV755 | Heavy chain | VRC-PG04b | WO2013090644 SEQ ID NO: 44 | 27739 |
| HIV756 | Heavy Chain | Vrc-pg20 | Zhou T et al., Immunity 39 (2), 245-258 (2013), | 27740 |
| | | | NCBI Accession # 4LSU_H (227aa) | |
| HIV757 | Heavy chain | X5 | U.S. Pat. No. 7,378,093B2 SEQ ID NO: 3 | 27741 |
| HIV758 | Heavy chain | X5 | U.S. Pat. No. 8,110,192B2 SEQ ID NO: 5 | 27742 |
| HIV759 | Heavy chain | X5 variant | U.S. Pat. No. 7,378,093B2 SEQ ID NO: 11 | 27743 |
| HIV760 | Heavy Chain | Z13e1 | Stanfield, R. L., et al, J. Mol. Biol. 414 (3). 460- | 27744 |
| | | | 476 (2011), NCBI Accession # 3Q1S_H(230aa) | |
| HIV761 | Heavy Chain | Z258- | Zhou. T et al., Structural Repertoire of HIV-1- | 27745 |
| | | vrc27.01 | Neutralizing Antibodies Targeting the CD4 | |
| | | | Supersite in 14 Donors; Cell 161 (6), 1280- | |
| | | | 1292 (2015), NCBI Accession # | |
| | | | 4YDI_H(227aa) | |
| HIV762 | Heavy Chain | | NCBI Accession # 1N0X_K (230aa) | 27746 |
| HIV763 | Heavy chain | | U.S. Pat. No. 5,804,440A SEQ ID NO: 142 | 27747 |
| HIV764 | Heavy chain | | U.S. Pat. No. 5,804,440A SEQ ID NO: 143 | 27748 |
| HIV765 | Heavy chain | | U.S. Pat. No. 5,804,440A SEQ ID NO: 144 | 27749 |
| HIV766 | Heavy chain | | U.S. Pat. No. 5,804,440A SEQ ID NO: 145 | 27750 |
| HIV767 | Heavy chain | | U.S. Pat. No. 5,804,440A SEQ ID NO: 146 | 27751 |
| HIV768 | Heavy chain | | U.S. Pat. No. 5,804,440A SEQ ID NO: 66 | 27752 |
| HIV769 | Heavy chain | | U.S. Pat. No. 5,804,440A SEQ ID NO: 67 | 27753 |
| HIV770 | Heavy chain | | U.S. Pat. No. 5,804,440A SEQ ID NO: 68 | 27754 |
| HIV771 | Heavy chain | | U.S. Pat. No. 5,804,440A SEQ ID NO: 70 | 27755 |
| HIV772 | Heavy chain | | U.S. Pat. No. 5,804,440A SEQ ID NO: 72 | 27756 |
| HIV773 | Heavy chain | | U.S. Pat. No. 5,804,440A SEQ ID NO: 73 | 27757 |
| HIV774 | Heavy chain | | U.S. Pat. No. 5,804,440A SEQ ID NO: 74 | 27758 |
| HIV775 | Heavy chain | | U.S. Pat. No. 5,804,440A SEQ ID NO: 75 | 27759 |
| HIV776 | Heavy chain | | U.S. Pat. No. 5,804,440A SEQ ID NO: 78 | 27760 |
| HIV777 | Heavy chain | | WO2014063059 SEQ ID NO: 10 | 27761 |
| HIV778 | Heavy chain | | WO2014063059 SEQ ID NO: 12 | 27762 |
| HIV779 | Heavy chain | | WO2014063059 SEQ ID NO: 130 | 27763 |
| HIV780 | Heavy chain | | WO2014063059 SEQ ID NO: 14 | 27764 |
| HIV781 | Heavy chain | | WO2014063059 SEQ ID NO: 16 | 27765 |
| HIV782 | Heavy chain | | WO2014063059 SEQ ID NO: 18 | 27766 |
| HIV783 | Heavy chain | | WO2014063059 SEQ ID NO: 20 | 27767 |
| HIV784 | Heavy chain | | WO2014063059 SEQ ID NO: 22 | 27768 |
| HIV785 | Heavy chain | | WO2014063059 SEQ ID NO: 24 | 27769 |
| HIV786 | Heavy chain | | WO2014063059 SEQ ID NO: 4 | 27770 |
| HIV787 | Heavy chain | | WO2014063059 SEQ ID NO: 6 | 27771 |
| HIV788 | Heavy chain | | WO2014063059 SEQ ID NO: 8 | 27772 |
| HIV789 | Heavy chain | | WO2014063059 SEQ ID NO: 2 | 27773 |
| | consensus | | | |
| HIV790 | Heavy chain constant | G4D | US20130195881 SEQ ID NO: 6 | 27774 |
| | region | | | |
| HIV791 | Heavy chain constant | G4H | US20130195881 SEQ ID NO: 5 | 27775 |
| | region | | | |
| HIV792 | Heavy chain constant | MV1 | US20130195881 SEQ ID NO: 7 | 27776 |
| | region | | | |
| HIV793 | Heavy chain constant | TNX-355, | US20130195881 SEQ ID NO: 4 | 27777 |
| | region | Idalizumab | | |
| HIV794 | Heavy Chain Fab | Ch04 | McLellan, J. S., et al. Nature 480 (7377), 336- | 27778 |
| | | | 343 (2011), NCBI Accession # 3U46_A | |
| | | | (238aa) | |
| HIV795 | Heavy Chain Of | 21C | Diskin, R., et al, Nat. Struct. Mol. Biol. 17 (5), | 27779 |
| | Anti-HIV Fab From | | 608-613 (2010), NCBI Accession # 3LMJ_H | |
| | Human 21c Antibody | | (231aa) | |
| HIV796 | Heavy Chain Of | 830a | Pan et al, J. Virol. 89 (15), 8003-8010 (2015), | 27780 |
| | Anti-hiv-1 Gp120 | | NCBI Accession # 4YWG_H (226aa) | |
| | V1v2 Antibody 830a | | | |
| HIV797 | Heavy chain partial | 412D | Huang C. et al “Structural basis of tyrosine | 27781 |
| | | | sulfation and VH-gene usage in antibodies that | |
| | | | recognize the HIV type 1 coreceptor-binding | |
| | | | site on gp120” Proc. Natl. Acad. Sci. U.S.A. | |
| | | | 101 (9), 2706-2711 (2004), NCBI Accession # | |
| | | | AAR88379 | |
| HIV798 | Heavy chain variable | 0.5γ(1C10) | U.S. Pat. No. 8,722,861B2 SEQ ID NO: 1 | 27782 |
| | region | | | |
| HIV799 | Heavy chain variable | 0.5δ (3D6) | U.S. Pat. No. 8,722,861B2 SEQ ID NO: 5 | 27783 |
| | region | | | |
| HIV800 | Heavy chain variable | 10J4 mAb | WO2015103549 SEQ ID NO: 3 | 27784 |
| | region | | | |
| HIV801 | Heavy chain variable | 10M6 mAb | WO2015103549 SEQ ID NO: 5 | 27785 |
| | region | | | |
| HIV802 | Heavy chain variable | 13110 mAb | WO2015103549 SEQ ID NO: 7 | 27786 |
| | region | | | |
| HIV803 | Heavy chain variable | 2N5mAb | WO2015103549 SEQ ID NO: 9 | 27787 |
| | region | | | |
| HIV804 | Heavy chain variable | 35022 mAb | WO2015103549 SEQ ID NO: 1 | 27788 |
| | region | | | |
| HIV805 | Heavy chain variable | 42F9 | U.S. Pat. No. 8,722,861B2 SEQ ID NO: 7 | 27789 |
| | region | | | |
| HIV806 | Heavy chain variable | 4835_F12 | US20140205612 SEQ ID NO: 404 | 27790 |
| | region | (PGT-124) | | |
| HIV807 | Heavy chain variable | 4838_L06 | US20140205612 SEQ ID NO: 66 | 27791 |
| | region | (PGT-121) | | |
| HIV808 | Heavy chain variable | 4858_P08 | US20140205612 SEQ ID NO: 167 | 27792 |
| | region | (PGT-123) | | |
| HIV809 | Heavy chain variable | 4869-K15 | US20140205612 SEQ ID NO: 419 | 27793 |
| | region | (PGT-133) | | |
| HIV810 | Heavy chain variable | 4873_E03 | US20140205612 SEQ ID NO: 62 | 27794 |
| | region | (PGT-121) | | |
| HIV811 | Heavy chain variable | 4876_M06 | US20140205612 SEQ ID NO: 434 | 27795 |
| | region | (PGT-134) | | |
| HIV812 | Heavy chain variable | 4877_D15 | US20140205612 SEQ ID NO: 155 | 27796 |
| | region | (PGT-122) | | |
| HIV813 | Heavy chain variable | 4964_G22 | US20140205612 SEQ ID NO: 275 | 27797 |
| | region | (PGT-141), | | |
| | | 4993_K13 | | |
| | | (PGT-141) | | |
| HIV814 | Heavy chain variable | 4970_K22 | US20140205612 SEQ ID NO: 306 | 27798 |
| | region | (PGT-144) | | |
| HIV815 | Heavy chain variable | 4980_N08 | US20140205612 SEQ ID NO: 297 | 27799 |
| | region | (PGT-143) | | |
| HIV816 | Heavy chain variable | 4995_E20 | US20140205612 SEQ ID NO: 291 | 27800 |
| | region | (PGT-142) | | |
| HIV817 | Heavy chain variable | 4995_P16 | US20140205612 SEQ ID NO: 400 | 27801 |
| | region | (PGT-145) | | |
| HIV818 | Heavy chain variable | 49G2 | U.S. Pat. No. 8,722,861B2 SEQ ID NO: 9 | 27802 |
| | region | | | |
| HIV819 | Heavy chain variable | 4O20mAb | WO2015103549 SEQ ID NO: 11 | 27803 |
| | region | | | |
| HIV820 | Heavy chain variable | 5114_A19 | US20140205612 SEQ ID NO: 333 | 27804 |
| | region | (PGT-128) | | |
| HIV821 | Heavy chain variable | 5120_N10 | US20140205612 SEQ ID NO: 462 | 27805 |
| | region | (PGT-139) | | |
| HIV822 | Heavy chain variable | 5131_A17 | US20140205612 SEQ ID NO: 443 | 27806 |
| | region | (PGT-132) | | |
| HIV823 | Heavy chain variable | 5136_H01 | US20140205612 SEQ ID NO: 345 | 27807 |
| | region | (PGT-131) | | |
| HIV824 | Heavy chain variable | 5138_G07 | US20140205612 SEQ ID NO: 453 | 27808 |
| | region | (PGT-138) | | |
| HIV825 | Heavy chain variable | 5141_B17 | US20140205612 SEQ ID NO: 199 | 27809 |
| | region | (PGT-126) | | |
| HIV826 | Heavy chain variable | 5145_B14 | US20140205612 SEQ ID NO: 318 | 27810 |
| | region | (PGT-127) | | |
| HIV827 | Heavy chain variable | 5147_N06 | US20140205612 SEQ ID NO: 215 | 27811 |
| | region | (PGT-130) | | |
| HIV828 | Heavy chain variable | 5329_C19 | US20140205612 SEQ ID NO: 248 | 27812 |
| | region | (PGT-136), | | |
| | | 5366_P21 | | |
| | | (PGT-136) | | |
| HIV829 | Heavy chain variable | 5343_B08 | US20140205612 SEQ ID NO: 231 | 27813 |
| | region | (PGT-135), | | |
| | | 5344_E16 | | |
| | | (PGT-135) | | |
| HIV830 | Heavy chain variable | 5345_I01 | US20140205612 SEQ ID NO: 362 | 27814 |
| | region | (PGT-137) | | |
| HIV831 | Heavy chain variable | 5G2 | U.S. Pat. No. 8,722,861B2 SEQ ID NO: 3 | 27815 |
| | region | | | |
| HIV832 | Heavy chain variable | 6808_B09 | US20140205612 SEQ ID NO: 546 | 27816 |
| | region | (PGT-156) | | |
| HIV833 | Heavy chain variable | 6831_A21 | US20140205612 SEQ ID NO: 473 | 27817 |
| | region | (PGT-151) | | |
| HIV834 | Heavy chain variable | 6843_G20 | US20140205612 SEQ ID NO: 516 | 27818 |
| | region | (PGT-154) | | |
| HIV835 | Heavy chain variable | 6881_N05 | US20140205612 SEQ ID NO: 572 | 27819 |
| | region | (PGT-158). | | |
| HIV836 | Heavy chain variable | 6889_117 | US20140205612 SEQ ID NO: 489 | 27820 |
| | region | (PGT-152) | | |
| HIV837 | Heavy chain variable | 6891_F06 | US20140205612 SEQ ID NO: 501 | 27821 |
| | region | (PGT-153) | | |
| HIV838 | Heavy chain variable | 6892_C23 | US20140205612 SEQ ID NO: 559 | 27822 |
| | region | (PGT-157) | | |
| HIV839 | Heavy chain variable | 6892_D19 | US20140205612 SEQ ID NO: 531 | 27823 |
| | region | (PGT-155) | | |
| HIV840 | Heavy chain variable | 7B9mAb | WO2015103549 SEQ ID NO: 13 | 27824 |
| | region | | | |
| HIV841 | Heavy chain variable | 7K3mAb | WO2015103549 SEQ ID NO: 15 | 27825 |
| | region | | | |
| HIV842 | Heavy chain variable | B4 | U.S. Pat. No. 7,872,110B2 SEQ ID NO: 2 | 27826 |
| | region | | | |
| HIV843 | Heavy chain variable | B4DIVHv.1 | U.S. Pat. No. 7,872,110B2 SEQ ID NO: 5 | 27827 |
| | region | | | |
| HIV844 | Heavy chain variable | B4DIVHv.2 | U.S. Pat. No. 7,872,110B2 SEQ ID NO: 6 | 27828 |
| | region | | | |
| HIV845 | Heavy chain variable | B4DTVHv.3 | U.S. Pat. No. 7,872,110B2 SEQ ID NO: 7 | 27829 |
| | region | | | |
| HIV846 | Heavy chain variable | B4DIVHv.4 | U.S. Pat. No. 7,872,110B2 SEQ ID NO: 8 | 27830 |
| | region | | | |
| HIV847 | Heavy chain variable | bI2 IgA2 | WO2014040024 SEQ ID NO: 29 | 27831 |
| | region | antibody | | |
| HIV848 | Heavy chain variable | CHμ39.1 | U.S. Pat. No. 5,773,247 SEQ ID NO: 10 | 27832 |
| | region | | | |
| HIV849 | Heavy chain variable | CHμ5.5 | U.S. Pat. No. 5,773,247 SEQ ID NO: 14 | 27833 |
| | region | | | |
| HIV850 | Heavy chain variable | F425-Alg8 | WO2014040024 SEQ ID NO: 9 | 27834 |
| | region | antibody | | |
| HIV851 | Heavy chain variable | Fab 43 | US20090191216 SEQ ID NO: 8 | 27835 |
| | region | | | |
| HIV852 | Heavy chain variable | HGN194 | US20110212106 SEQ ID NO: 45 | 27836 |
| | region | | | |
| HIV853 | Heavy chain variable | HJ16 | US20110212106 SEQ ID NO: 13 | 27837 |
| | region | | | |
| HIV854 | Heavy chain variable | HK20 | US20110212106 SEQ ID NO: 29 | 27838 |
| | region | | | |
| HIV855 | Heavy chain variable | IgA antibody | WO2014040024 SEQ ID NO: 11 | 27839 |
| | region | | | |
| HIV856 | Heavy chain variable | L1719A11 | US20150158934 SEQ ID NO: 175 | 27840 |
| | region | | | |
| HIV857 | Heavy chain variable | L1719A12 | US20150158934 SEQ ID NO: 176 | 27841 |
| | region | | | |
| HIV858 | Heavy chain variable | L1719A9 | US20150158934 SEQ ID NO: 174 | 27842 |
| | region | | | |
| HIV859 | Heavy chain variable | L1719B12 | US20150158934 SEQ ID NO: 177 | 27843 |
| | region | | | |
| HIV860 | Heavy chain variable | L1719C1 | US20150158934 SEQ ID NO: 178 | 27844 |
| | region | | | |
| HIV861 | Heavy chain variable | L1719D10 | US20150158934 SEQ ID NO: 179 | 27845 |
| | region | | | |
| HIV862 | Heavy chain variable | L1719E1 | US20150158934 SEQ ID NO: 180 | 27846 |
| | region | | | |
| HIV863 | Heavy chain variable | L1719E11 | US20150158934 SEQ ID NO: 181 | 27847 |
| | region | | | |
| HIV864 | Heavy chain variable | L1719E12 | US20150158934 SEQ ID NO: 182 | 27848 |
| | region | | | |
| HIV865 | Heavy chain variable | L1719F11 | US20150158934 SEQ ID NO: 183 | 27849 |
| | region | | | |
| HIV866 | Heavy chain variable | L1719H10 | US20150158934 SEQ ID NO: 185 | 27850 |
| | region | | | |
| HIV867 | Heavy chain variable | L1719H9 | US20150158934 SEQ ID NO: 184 | 27851 |
| | region | | | |
| HIV868 | Heavy chain variable | L1720C1 | US20150158934 SEQ ID NO: 186 | 27852 |
| | region | | | |
| HIV869 | Heavy chain variable | L1720E4 | US20150158934 SEQ ID NO: 187 | 27853 |
| | region | | | |
| HIV870 | Heavy chain variable | L1721A3 | US20150158934 SEQ ID NO: 188 | 27854 |
| | region | | | |
| HIV871 | Heavy chain variable | L1721A5 | US20150158934 SEQ ID NO: 189 | 27855 |
| | region | | | |
| HIV872 | Heavy chain variable | L1721A8 | US20150158934 SEQ ID NO: 190 | 27856 |
| | region | | | |
| HIV873 | Heavy chain variable | L1721H4 | US20150158934 SEQ ID NO: 191 | 27857 |
| | region | | | |
| HIV874 | Heavy chain variable | L1723A10 | US20150158934 SEQ ID NO: 193 | 27858 |
| | region | | | |
| HIV875 | Heavy chain variable | L1723A11 | US20150158934 SEQ ID NO: 194 | 27859 |
| | region | | | |
| HIV876 | Heavy chain variable | L1723A9 | US20150158934 SEQ ID NO: 192 | 27860 |
| | region | | | |
| HIV877 | Heavy chain variable | L1723E5 | US20150158934 SEQ ID NO: 195 | 27861 |
| | region | | | |
| HIV878 | Heavy chain variable | L2319G11 | US20150158934 SEQ ID NO: 197 | 27862 |
| | region | | | |
| HIV879 | Heavy chain variable | L2319G7 | US20150158934 SEQ ID NO: 196 | 27863 |
| | region | | | |
| HIV880 | Heavy chain variable | L2319H7 | US20150158934 SEQ ID NO: 198 | 27864 |
| | region | | | |
| HIV881 | Heavy chain variable | L2320E9 | US20150158934 SEQ ID NO: 199 | 27865 |
| | region | | | |
| HIV882 | Heavy chain variable | L2320F9 | US20150158934 SEQ ID NO: 200 | 27866 |
| | region | | | |
| HIV883 | Heavy chain variable | L2321B7 | US20150158934 SEQ ID NO: 201 | 27867 |
| | region | | | |
| HIV884 | Heavy chain variable | L2321H6 | US20150158934 SEQ ID NO: 202 | 27868 |
| | region | | | |
| HIV885 | Heavy chain variable | L81C11 | US20150158934 SEQ ID NO: 15 | 27869 |
| | region | | | |
| HIV886 | Heavy chain variable | L81C9 | US20150158934 SEQ ID NO: 30 | 27870 |
| | region | | | |
| HIV887 | Heavy chain variable | L81D9 | US20150158934 SEQ ID NO: 10 | 27871 |
| | region | | | |
| HIV888 | Heavy chain variable | L81E1 | US20150158934 SEQ ID NO: 18 | 27872 |
| | region | | | |
| HIV889 | Heavy chain variable | L81E7 | US20150158934 SEQ ID NO: 16 | 27873 |
| | region | | | |
| HIV890 | Heavy chain variable | L81F1 | US20150158934 SEQ ID NO: 19 | 27874 |
| | region | | | |
| HIV891 | Heavy chain variable | L81G7 | US20150158934 SEQ ID NO: 13 | 27875 |
| | region | | | |
| HIV892 | Heavy chain variable | L81H1 | US20150158934 SEQ ID NO: 98 | 27876 |
| | region | | | |
| HIV893 | Heavy chain variable | L81H2 | US20150158934 SEQ ID NO: 23 | 27877 |
| | region | | | |
| HIV894 | Heavy chain variable | L81H7 | US20150158934 SEQ ID NO: 11 | 27878 |
| | region | | | |
| HIV895 | Heavy chain variable | L81H9 | US20150158934 SEQ ID NO: 28 | 27879 |
| | region | | | |
| HIV896 | Heavy chain variable | L82B12A | US20150158934 SEQ ID NO: 105 | 27880 |
| | region | | | |
| HIV897 | Heavy chain variable | L82B1A | US20150158934 SEQ ID NO: 99 | 27881 |
| | region | | | |
| HIV898 | Heavy chain variable | L82B1D | US20150158934 SEQ ID NO: 100 | 27882 |
| | region | | | |
| HIV899 | Heavy chain variable | L82B2A | US20150158934 SEQ ID NO: 101 | 27883 |
| | region | | | |
| HIV900 | Heavy chain variable | L82B3F | US20150158934 SEQ ID NO: 102 | 27884 |
| | region | | | |
| HIV901 | Heavy chain variable | L82B4A | US20150158934 SEQ ID NO: 103 | 27885 |
| | region | | | |
| HIV902 | Heavy chain variable | L82B4E | US20150158934 SEQ ID NO: 104 | 27886 |
| | region | | | |
| HIV903 | Heavy chain variable | L82B4F | US20150158934 SEQ ID NO: 21 | 27887 |
| | region | | | |
| HIV904 | Heavy chain variable | L832G6 | US20150158934 SEQ ID NO: 113 | 27888 |
| | region | | | |
| HIV905 | Heavy chain variable | L833E1 | US20150158934 SEQ ID NO: 72 | 27889 |
| | region | | | |
| HIV906 | Heavy chain variable | L833F5 | US20150158934 SEQ ID NO: 17 | 27890 |
| | region | | | |
| HIV907 | Heavy chain variable | L833H1 | US20150158934 SEQ ID NO: 114 | 27891 |
| | region | | | |
| HIV908 | Heavy chain variable | L833H3 | US20150158934 SEQ ID NO: 115 | 27892 |
| | region | | | |
| HIV909 | Heavy chain variable | L88B10B | US20150158934 SEQ ID NO: 27 | 27893 |
| | region | | | |
| HIV910 | Heavy chain variable | L88B11B | US20150158934 SEQ ID NO: 12 | 27894 |
| | region | | | |
| HIV911 | Heavy chain variable | L88B12G | US20150158934 SEQ ID NO: 29 | 27895 |
| | region | | | |
| HIV912 | Heavy chain variable | L88B1D | US20150158934 SEQ ID NO: 20 | 27896 |
| | region | | | |
| HIV913 | Heavy chain variable | L88B2A | US20150158934 SEQ ID NO: 106 | 27897 |
| | region | | | |
| HIV914 | Heavy chain variable | L88FA2 | US20150158934 SEQ ID NO: 26 | 27898 |
| | region | | | |
| HIV915 | Heavy chain variable | L88FA3 | US20150158934 SEQ ID NO: 107 | 27899 |
| | region | | | |
| HIV916 | Heavy chain variable | L88FA5 | US20150158934 SEQ ID NO: 108 | 27900 |
| | region | | | |
| HIV917 | Heavy chain variable | L88FB1 | US20150158934 SEQ ID NO: 25 | 27901 |
| | region | | | |
| HIV918 | Heavy chain variable | L88FC11 | US20150158934 SEQ ID NO: 22 | 27902 |
| | region | | | |
| HIV919 | Heavy chain variable | L88FD12 | US20150158934 SEQ ID NO: 24 | 27903 |
| | region | | | |
| HIV920 | Heavy chain variable | L89B12D | US20150158934 SEQ ID NO: 112 | 27904 |
| | region | | | |
| HIV921 | Heavy chain variable | L89B1D | US20150158934 SEQ ID NO: 109 | 27905 |
| | region | | | |
| HIV922 | Heavy chain variable | L89B2C | US20150158934 SEQ ID NO: 110 | 27906 |
| | region | | | |
| HIV923 | Heavy chain variable | L89B3E | US20150158934 SEQ ID NO: 14 | 27907 |
| | region | | | |
| HIV924 | Heavy chain variable | L89B6B | US20150158934 SEQ ID NO: 111 | 27908 |
| | region | | | |
| HIV925 | Heavy chain variable | L8Cb15 | US20150158934 SEQ ID NO: 116 | 27909 |
| | region | | | |
| HIV926 | Heavy chain variable | L8Cj3 | US20150158934 SEQ ID NO: 73 | 27910 |
| | region | | | |
| HIV927 | Heavy chain variable | L8Fe2 | US20150158934 SEQ ID NO: 117 | 27911 |
| | region | | | |
| HIV928 | Heavy chain variable | L8Fg12 | US20150158934 SEQ ID NO: 118 | 27912 |
| | region | | | |
| HIV929 | Heavy chain variable | L8Fj19 | US20150158934 SEQ ID NO: 119 | 27913 |
| | region | | | |
| HIV930 | Heavy chain variable | L8Fo17 | US20150158934 SEQ ID NO: 120 | 27914 |
| | region | | | |
| HIV931 | Heavy chain variable | L8Fp6 | US20150158934 SEQ ID NO: 121 | 27915 |
| | region | | | |
| HIV932 | Heavy chain variable | L8Hi20 | US20150158934 SEQ ID NO: 122 | 27916 |
| | region | | | |
| HIV933 | Heavy chain variable | L911B11E | US20150158934 SEQ ID NO: 140 | 27917 |
| | region | | | |
| HIV934 | Heavy chain variable | L911B12B | US20150158934 SEQ ID NO: 71 | 27918 |
| | region | | | |
| HIV935 | Heavy chain variable | L911B1E | US20150158934 SEQ ID NO: 137 | 27919 |
| | region | | | |
| HIV936 | Heavy chain variable | L911B1G | US20150158934 SEQ ID NO: 65 | 27920 |
| | region | | | |
| HIV937 | Heavy chain variable | L911B2E | US20150158934 SEQ ID NO: 138 | 27921 |
| | region | | | |
| HIV938 | Heavy chain variable | L911B3D | US20150158934 SEQ ID NO: 75 | 27922 |
| | region | | | |
| HIV939 | Heavy chain variable | L911B9A | US20150158934 SEQ ID NO: 139 | 27923 |
| | region | | | |
| HIV940 | Heavy chain variable | L911F12B | US20150158934 SEQ ID NO: 142 | 27924 |
| | region | | | |
| HIV941 | Heavy chain variable | L911F1B | US20150158934 SEQ ID NO: 141 | 27925 |
| | region | | | |
| HIV942 | Heavy chain variable | L911F1F | US20150158934 SEQ ID NO: 77 | 27926 |
| | region | | | |
| HIV943 | Heavy chain variable | L911F4C | US20150158934 SEQ ID NO: 33 | 27927 |
| | region | | | |
| HIV944 | Heavy chain variable | L91A1 | US20150158934 SEQ ID NO: 123 | 27928 |
| | region | | | |
| HIV945 | Heavy chain variable | L91B5 | US20150158934 SEQ ID NO: 37 | 27929 |
| | region | | | |
| HIV946 | Heavy chain variable | L91B5, 4A7 | US20150158934 SEQ ID NO: 97 | 27930 |
| | region | | | |
| HIV947 | Heavy chain variable | L91B5, A12 | US20150158934 SEQ ID NO: 92 | 27931 |
| | region | | | |
| HIV948 | Heavy chain variable | L91B5, A4 | US20150158934 SEQ ID NO: 90 | 27932 |
| | region | | | |
| HIV949 | Heavy chain variable | L91B5, A7 | US20150158934 SEQ ID NO: 91 | 27933 |
| | region | | | |
| HIV950 | Heavy chain variable | L91B5, B2 | US20150158934 SEQ ID NO: 93 | 27934 |
| | region | | | |
| HIV951 | Heavy chain variable | L91B5, D4 | US20150158934 SEQ ID NO: 94 | 27935 |
| | region | | | |
| HIV952 | Heavy chain variable | L91B5, F11 | US20150158934 SEQ ID NO: 96 | 27936 |
| | region | | | |
| HIV953 | Heavy chain variable | L91B5, F4 | US20150158934 SEQ ID NO: 95 | 27937 |
| | region | | | |
| HIV954 | Heavy chain variable | L91C2 | US20150158934 SEQ ID NO: 61 | 27938 |
| | region | | | |
| HIV955 | Heavy chain variable | L91E1 | US20150158934 SEQ ID NO: 45 | 27939 |
| | region | | | |
| HIV956 | Heavy chain variable | L91E2 | US20150158934 SEQ ID NO: 124 | 27940 |
| | region | | | |
| HIV957 | Heavy chain variable | L91F10 | US20150158934 SEQ ID NO: 69 | 27941 |
| | region | | | |
| HIV958 | Heavy chain variable | L91G2 | US20150158934 SEQ ID NO: 64 | 27942 |
| | region | | | |
| HIV959 | Heavy chain variable | L91H3 | US20150158934 SEQ ID NO: 128 | 27943 |
| | region | | | |
| HIV960 | Heavy chain variable | L91H9 | US20150158934 SEQ ID NO: 41 | 27944 |
| | region | | | |
| HIV961 | Heavy chain variable | L922B2 | US20150158934 SEQ ID NO: 143 | 27945 |
| | region | | | |
| HIV962 | Heavy chain variable | L922B4 | US20150158934 SEQ ID NO: 144 | 27946 |
| | region | | | |
| HIV963 | Heavy chain variable | L922E1 | US20150158934 SEQ ID NO: 145 | 27947 |
| | region | | | |
| HIV964 | Heavy chain variable | L922E2 | US20150158934 SEQ ID NO: 53 | 27948 |
| | region | | | |
| HIV965 | Heavy chain variable | L923A1 | US20150158934 SEQ ID NO: 146 | 27949 |
| | region | | | |
| HIV966 | Heavy chain variable | L923A4 | US20150158934 SEQ ID NO: 32 | 27950 |
| | region | | | |
| HIV967 | Heavy chain variable | L92A11 | US20150158934 SEQ ID NO: 125 | 27951 |
| | region | | | |
| HIV968 | Heavy chain variable | L92C7 | US20150158934 SEQ ID NO: 62 | 27952 |
| | region | | | |
| HIV969 | Heavy chain variable | L92D4 | US20150158934 SEQ ID NO: 126 | 27953 |
| | region | | | |
| HIV970 | Heavy chain variable | L92E6 | US20150158934 SEQ ID NO: 63 | 27954 |
| | region | | | |
| HIV971 | Heavy chain variable | L92E7 | US20150158934 SEQ ID NO: 74 | 27955 |
| | region | | | |
| HIV972 | Heavy chain variable | L92E7, A1 | US20150158934 SEQ ID NO: 85 | 27956 |
| | region | | | |
| HIV973 | Heavy chain variable | L92E7, A2 | US20150158934 SEQ ID NO: 86 | 27957 |
| | region | | | |
| HIV974 | Heavy chain variable | L92E7, A3 | US20150158934 SEQ ID NO: 87 | 27958 |
| | region | | | |
| HIV975 | Heavy chain variable | L92E7, A4 | US20150158934 SEQ ID NO: 80 | 27959 |
| | region | | | |
| HIV976 | Heavy chain variable | L92E7, A4 | US20150158934 SEQ ID NO: 88 | 27960 |
| | region | | | |
| HIV977 | Heavy chain variable | L92E7, A5 | US20150158934 SEQ ID NO: 89 | 27961 |
| | region | | | |
| HIV978 | Heavy chain variable | L92E7, B5 | US20150158934 SEQ ID NO: 78 | 27962 |
| | region | | | |
| HIV979 | Heavy chain variable | L92E7. C | US20150158934 SEQ ID NO: 79 | 27963 |
| | region | | | |
| HIV980 | Heavy chain variable | L92E7, C3 | US20150158934 SEQ ID NO: 82 | 27964 |
| | region | | | |
| HIV981 | Heavy chain variable | L92E7, D3 | US20150158934 SEQ ID NO: 83 | 27965 |
| | region | | | |
| HIV982 | Heavy chain variable | L92E7, E1 | US20150158934 SEQ ID NO: 84 | 27966 |
| | region | | | |
| HIV983 | Heavy chain variable | L92E7, G4 | US20150158934 SEQ ID NO: 81 | 27967 |
| | region | | | |
| HIV984 | Heavy chain variable | L932A9 | US20150158934 SEQ ID NO: 58 | 27968 |
| | region | | | |
| HIV985 | Heavy chain variable | L932E10 | US20150158934 SEQ ID NO: 35 | 27969 |
| | region | | | |
| HIV986 | Heavy chain variable | L932E8 | US20150158934 SEQ ID NO: 147 | 27970 |
| | region | | | |
| HIV987 | Heavy chain variable | L932G9 | US20150158934 SEQ ID NO: 34 | 27971 |
| | region | | | |
| HIV988 | Heavy chain variable | L933D10 | US20150158934 SEQ ID NO: 50 | 27972 |
| | region | | | |
| HIV989 | Heavy chain variable | L93B3 | US20150158934 SEQ ID NO: 70 | 27973 |
| | region | | | |
| HIV990 | Heavy chain variable | L93B4 | US20150158934 SEQ ID NO: 127 | 27974 |
| | region | | | |
| HIV991 | Heavy chain variable | L93C3 | US20150158934 SEQ ID NO: 51 | 27975 |
| | region | | | |
| HIV992 | Heavy chain variable | L93C6 | US20150158934 SEQ ID NO: 67 | 27976 |
| | region | | | |
| HIV993 | Heavy chain variable | L93D3 | US20150158934 SEQ ID NO: 129 | 27977 |
| | region | | | |
| HIV994 | Heavy chain variable | L93D4 | US20150158934 SEQ ID NO: 43 | 27978 |
| | region | | | |
| HIV995 | Heavy chain variable | L93D9 | US20150158934 SEQ ID NO: 130 | 27979 |
| | region | | | |
| HIV996 | Heavy chain variable | L93E3 | US20150158934 SEQ ID NO: 55 | 27980 |
| | region | | | |
| HIV997 | Heavy chain variable | L93E6 | US20150158934 SEQ ID NO: 131 | 27981 |
| | region | | | |
| HIV998 | Heavy chain variable | L93F12 | US20150158934 SEQ ID NO: 133 | 27982 |
| | region | | | |
| HIV999 | Heavy chain variable | L93F2 | US20150158934 SEQ ID NO: 132 | 27983 |
| | region | | | |
| HIV1000 | Heavy chain variable | L93F2 | US20150158934 SEQ ID NO: 59 | 27984 |
| | region | | | |
| HIV1001 | Heavy chain variable | L93H6 | US20150158934 SEQ ID NO: 38 | 27985 |
| | region | | | |
| HIV1002 | Heavy chain variable | L93H9 | US20150158934 SEQ ID NO: 134 | 27986 |
| | region | | | |
| HIV1003 | Heavy chain variable | L94A12 | US20150158934 SEQ ID NO: 46 | 27987 |
| | region | | | |
| HIV1004 | Heavy chain variable | L94C2 | US20150158934 SEQ ID NO: 31 | 27988 |
| | region | | | |
| HIV1005 | Heavy chain variable | L94D12 | US20150158934 SEQ ID NO: 42 | 27989 |
| | region | | | |
| HIV1006 | Heavy chain variable | L94D4 | US20150158934 SEQ ID NO: 47 | 27990 |
| | region | | | |
| HIV1007 | Heavy chain variable | L94E3 | US20150158934 SEQ ID NO: 39 | 27991 |
| | region | | | |
| HIV1008 | Heavy chain variable | L94E4 | US20150158934 SEQ ID NO: 54 | 27992 |
| | region | | | |
| HIV1009 | Heavy chain variable | L94E5 | US20150158934 SEQ ID NO: 57 | 27993 |
| | region | | | |
| HIV1010 | Heavy chain variable | L94H1 | US20150158934 SEQ ID NO: 36 | 27994 |
| | region | | | |
| HIV1011 | Heavy chain variable | L94H2 | US20150158934 SEQ ID NO: 40 | 27995 |
| | region | | | |
| HIV1012 | Heavy chain variable | L94H5 | US20150158934 SEQ ID NO: 48 | 27996 |
| | region | | | |
| HIV1013 | Heavy chain variable | L94H7 | US20150158934 SEQ ID NO: 135 | 27997 |
| | region | | | |
| HIV1014 | Heavy chain variable | L95B10D | US20150158934 SEQ ID NO: 136 | 27998 |
| | region | | | |
| HIV1015 | Heavy chain variable | L95B12A | US20150158934 SEQ ID NO: 68 | 27999 |
| | region | | | |
| HIV1016 | Heavy chain variable | L95B12E | US20150158934 SEQ ID NO: 66 | 28000 |
| | region | | | |
| HIV1017 | Heavy chain variable | L95B8A | US20150158934 SEQ ID NO: 60 | 28001 |
| | region | | | |
| HIV1018 | Heavy chain variable | L98FB10 | US20150158934 SEQ ID NO: 76 | 28002 |
| | region | | | |
| HIV1019 | Heavy chain variable | L9Ab16 | US20150158934 SEQ ID NO: 148 | 28003 |
| | region | | | |
| HIV1020 | Heavy chain variable | L9Ab19 | US20150158934 SEQ ID NO: 149 | 28004 |
| | region | | | |
| HIV1021 | Heavy chain variable | L9Ad13 | US20150158934 SEQ ID NO: 151 | 28005 |
| | region | | | |
| HIV1022 | Heavy chain variable | L9Ad14 | US20150158934 SEQ ID NO: 152 | 28006 |
| | region | | | |
| HIV1023 | Heavy chain variable | L9Ad3 | US20150158934 SEQ ID NO: 150 | 28007 |
| | region | | | |
| HIV1024 | Heavy chain variable | L9Aj2 | US20150158934 SEQ ID NO: 153 | 28008 |
| | region | | | |
| HIV1025 | Heavy chain variable | L9An7 | US20150158934 SEQ ID NO: 154 | 28009 |
| | region | | | |
| HIV1026 | Heavy chain variable | L9Ao15 | US20150158934 SEQ ID NO: 155 | 28010 |
| | region | | | |
| HIV1027 | Heavy chain variable | L9Ap11 | US20150158934 SEQ ID NO: 156 | 28011 |
| | region | | | |
| HIV1028 | Heavy chain variable | L9Bb3 | US20150158934 SEQ ID NO: 157 | 28012 |
| | region | | | |
| HIV1029 | Heavy chain variable | L9Bc6 | US20150158934 SEQ ID NO: 158 | 28013 |
| | region | | | |
| HIV1030 | Heavy chain variable | L9Bd8 | US20150158934 SEQ ID NO: 159 | 28014 |
| | region | | | |
| HIV1031 | Heavy chain variable | L9Bd9 | US20150158934 SEQ ID NO: 160 | 28015 |
| | region | | | |
| HIV1032 | Heavy chain variable | L9Be11 | US20150158934 SEQ ID NO: 161 | 28016 |
| | region | | | |
| HIV1033 | Heavy chain variable | L9Bf11 | US20150158934 SEQ ID NO: 49 | 28017 |
| | region | | | |
| HIV1034 | Heavy chain variable | L9Bf19 | US20150158934 SEQ ID NO: 162 | 28018 |
| | region | | | |
| HIV1035 | Heavy chain variable | L9Bj13 | US20150158934 SEQ ID NO: 163 | 28019 |
| | region | | | |
| HIV1036 | Heavy chain variable | L9Bm10 | US20150158934 SEQ ID NO: 164 | 28020 |
| | region | | | |
| HIV1037 | Heavy chain variable | L9Bm16 | US20150158934 SEQ ID NO: 56 | 28021 |
| | region | | | |
| HIV1038 | Heavy chain variable | L9Bp16 | US20150158934 SEQ ID NO: 165 | 28022 |
| | region | | | |
| HIV1039 | Heavy chain variable | L9Bp5 | US20150158934 SEQ ID NO: 44 | 28023 |
| | region | | | |
| HIV1040 | Heavy chain variable | L9Ca12 | US20150158934 SEQ ID NO: 166 | 28024 |
| | region | | | |
| HIV1041 | Heavy chain variable | L9Ca13 | US20150158934 SEQ ID NO: 167 | 28025 |
| | region | | | |
| HIV1042 | Heavy chain variable | L9Cd12 | US20150158934 SEQ ID NO: 168 | 28026 |
| | region | | | |
| HIV1043 | Heavy chain variable | L9Cf15 | US20150158934 SEQ ID NO: 169 | 28027 |
| | region | | | |
| HIV1044 | Heavy chain variable | L9Cl22 | US20150158934 SEQ ID NO: 52 | 28028 |
| | region | | | |
| HIV1045 | Heavy chain variable | L9Cm18 | US20150158934 SEQ ID NO: 170 | 28029 |
| | region | | | |
| HIV1046 | Heavy chain variable | L9Co22 | US20150158934 SEQ ID NO: 171 | 28030 |
| | region | | | |
| HIV1047 | Heavy chain variable | L9Cp5 | US20150158934 SEQ ID NO: 172 | 28031 |
| | region | | | |
| HIV1048 | Heavy chain variable | L9Cpl3 | US20150158934 SEQ ID NO: 173 | 28032 |
| | region | | | |
| HIV1049 | Heavy chain variable | Makandal | US20100111990 SEQ ID NO: 4 | 28033 |
| | region | monoclonal | | |
| | | antibody | | |
| | | (Mmab) | | |
| HIV1050 | Heavy chain variable | NM-01 | U.S. Pat. No. 5,665,569 SEQ ID NO: 17 | 28034 |
| | region | | | |
| HIV1051 | Heavy chain variable | NM-01 | U.S. Pat. No. 5,665,569 SEQ ID NO: 27 | 28035 |
| | region | HuVH | | |
| HIV1052 | Heavy chain variable | NM-01 | U.S. Pat. No. 5,665,569 SEQ ID NO: 29 | 28036 |
| | region | HuVK | | |
| HIV1053 | Heavy chain variable | NM-01 | U.S. Pat. No. 5,665,569 SEQ ID NO: 31 | 28037 |
| | region | HuVKF | | |
| HIV1054 | Heavy chain variable | PGT125 | Walker L. M. et al “Broad neutralization | 28038 |
| | region | | coverage of HIV by multiple highly potent | |
| | | | antibodies”, Nature 477 (7365), 466-470 | |
| | | | (2011), NCBI Accession # AEN14393 | |
| HIV1055 | Heavy chain variable | PGT126 | Walker L. M. et al “Broad neutralization | 28039 |
| | region | | coverage of HIV by multiple highly potent | |
| | | | antibodies”, Nature 477 (7365), 466-470 | |
| | | | (2011), NCBI Accession # AEN14394 | |
| HIV1056 | Heavy chain variable | PGT131 | Walker L. M. et al “Broad neutralization | 28040 |
| | region | | coverage of HIV by multiple highly potent | |
| | | | antibodies”, Nature 477 (7365), 466-470 | |
| | | | (2011), NCBI Accession # AEN14389 | |
| HIV1057 | Heavy chain variable | PGT136 | Walker L. M. et al “Broad neutralization | 28041 |
| | region | | coverage of HIV by multiple highly potent | |
| | | | antibodies”, Nature 477 (7365),466-470 | |
| | | | (2011), NCBI Accession # AEN14400 | |
| HIV1058 | Heavy chain variable | PGT137 | Walker L. M. et al “Broad neutralization | 28042 |
| | region | | coverage of HIV by multiple highly potent | |
| | | | antibodies”, Nature 477 (7365), 466-470 | |
| | | | (2011), NCBI Accession # AEN14401 | |
| HIV1059 | Heavy chain variable | PGT141 | Walker L. M. et al “Broad neutralization | 28043 |
| | region | | coverage of HIV by multiple highly potent | |
| | | | antibodies”, Nature 477 (7365), 466-470 | |
| | | | (2011), NCBI Accession # AEN14402 | |
| HIV1060 | Heavy chain variable | PGT142 | Walker L. M. et al “Broad neutralization | 28044 |
| | region | | coverage of HIV by multiple highly potent | |
| | | | antibodies”, Nature 477 (7365), 466-470 | |
| | | | (2011), NCBI Accession # AEN14368 | |
| HIV1061 | Heavy chain variable | PGT143 | Walker L. M. et al “Broad neutralization | 28045 |
| | region | | coverage of HIV by multiple highly potent | |
| | | | antibodies”, Nature 477 (7365), 466-470 | |
| | | | (2011), NCBI Accession # AEN14404 | |
| HIV1062 | Heavy chain variable | PGT144 | Walker L. M. et al “Broad neutralization | 28046 |
| | region | | coverage of HIV by multiple highly potent | |
| | | | antibodies”, Nature 477 (7365), 466-470 | |
| | | | (2011), NCBI Accession # AEN14405 | |
| HIV1063 | Heavy chain variable | PGT151 | Falkowska, E. et al “Broadly Neutralizing HIV | 28047 |
| | region | | Antibodies Define a Glycan-Dependent Epitope | |
| | | | on the Perfusion Conformation of gp41 on | |
| | | | Cleaved Envelope Trimers” Immunity 40 (5), | |
| | | | 657-668 (2014), NCBI Accession # AIC3535 | |
| HIV1064 | Heavy chain variable | PGT152 | Falkowska, E. et al “Broadly Neutralizing HIV | 28048 |
| | region | | Antibodies Define a Glycan-Dependent Epitope | |
| | | | on the Perfusion Conformation of gp41 on | |
| | | | Cleaved Envelope Trimers” Immunity 40 (5), | |
| | | | 657-668 (2014), NCBI Accession # AIC32536 | |
| HIV1065 | Heavy chain variable | PGT153 | Falkowska, E. et al “Broadly Neutralizing HIV | 28049 |
| | region | | Antibodies Define a Glycan-Dependent Epitope | |
| | | | on the Perfusion Conformation of gp41 on | |
| | | | Cleaved Envelope Trimers” Immunity 40 (5), | |
| | | | 657-668 (2014), NCBI Accession # AIC32537 | |
| HIV1066 | Heavy chain variable | PGT154 | Falkowska, E. et al “Broadly Neutralizing HIV | 28050 |
| | region | | Antibodies Define a Glycan-Dependent Epitope | |
| | | | on the Perfusion Conformation of gp41 on | |
| | | | Cleaved Envelope Trimers” Immunity 40 (5), | |
| | | | 657-668 (2014), NCBI Accession # AIC32521 | |
| HIV1067 | Heavy chain variable | PGT155 | Falkowska, E. et al “Broadly Neutralizing HIV | 28051 |
| | region | | Antibodies Define a Glycan-Dependent Epitope | |
| | | | on the Perfusion Conformation of gp41 on | |
| | | | Cleaved Envelope Trimers” Immunity 40 (5), | |
| | | | 657-668 (2014), NCBI Accession # AIC32539 | |
| HIV1068 | Heavy chain variable | PGT156 | Falkowska, E. et al “Broadly Neutralizing HIV | 28052 |
| | region | | Antibodies Define a Glycan-Dependent Epitope | |
| | | | on the Perfusion Conformation of gp41 on | |
| | | | Cleaved Envelope Trimers” Immunity 40 (5), | |
| | | | 657-668 (2014), NCBI Accession # AIC32540 | |
| HIV1069 | Heavy chain variable | PGT157 | Falkowska, E. et al “Broadly Neutralizing HIV | 28053 |
| | region | | Antibodies Define a Glycan-Dependent Epitope | |
| | | | on the Perfusion Conformation of gp41 on | |
| | | | Cleaved Envelope Trimers” Immunity 40 (5), | |
| | | | 657-668 (2014), NCBI Accession # AIC32541 | |
| HIV1070 | Heavy chain variable | PGT158 | Falkowska, E. et al “Broadly Neutralizing HIV | 28054 |
| | region | | Antibodies Define a Glycan-Dependent Epitope | |
| | | | on the Perfusion Conformation of gp41 on | |
| | | | Cleaved Envelope Trimers” Immunity 40 (5), | |
| | | | 657-668 (2014), NCBI Accession # AIC32542 | |
| HIV1071 | Heavy chain variable | rF105 | WO1993012232 SEQ ID NO: 4 | 28055 |
| | region | | | |
| HIV1072 | Heavy chain variable | ScFvX5- | U.S. Pat. No. 7,378,093B2 SEQ ID NO: 14 | 28056 |
| | region | CD4 | | |
| HIV1073 | Heavy chain variable | TNX-355, | US20130195881 SEQ ID NO: 3 | 28057 |
| | region | Idalizumab | | |
| HIV1074 | Heavy chain variable | VCR14 | US20150044137 SEQ ID NO: 13 | 28058 |
| | region | | | |
| HIV1075 | Heavy chain variable | VCR14b | US20150044137 SEQ ID NO: 14 | 28059 |
| | region | | | |
| HIV1076 | Heavy chain variable | VCR14c | US20150044137 SEQ ID NO: 15 | 28060 |
| | region | | | |
| HIV1077 | Heavy chain variable | VCR16 | US20150044137 SEQ ID NO: 29 | 28061 |
| | region | | | |
| HIV1078 | Heavy chain variable | VCR16b | US20150044137 SEQ ID NO: 30 | 28062 |
| | region | | | |
| HIV1079 | Heavy chain variable | VCR16c | US20150044137 SEQ ID NO: 31 | 28063 |
| | region | | | |
| HIV1080 | Heavy chain variable | VCR16d | US20150044137 SEQ ID NO: 32 | 28064 |
| | region | | | |
| HIV1081 | Heavy chain variable | VLP_A14 | US20150158934 SEQ ID NO: 203 | 28065 |
| | region | | | |
| HIV1082 | Heavy chain variable | VLP_B9 | US20150158934 SEQ ID NO: 204 | 28066 |
| | region | | | |
| HIV1083 | Heavy chain variable | VLP3_B21 | US20150158934 SEQ ID NO: 205 | 28067 |
| | region | | | |
| HIV1084 | Heavy chain variable | VRC13 | US20150044137 SEQ ID NO: 5 | 28068 |
| | region | | | |
| HIV1085 | Heavy chain variable | VRC13b | US20150044137 SEQ ID NO: 6 | 28069 |
| | region | | | |
| HIV1086 | Heavy chain variable | VRC13c | US20150044137 SEQ ID NO: 7 | 28070 |
| | region | | | |
| HIV1087 | Heavy chain variable | VRC13d | US20150044137 SEQ ID NO: 8 | 28071 |
| | region | | | |
| HIV1088 | Heavy chain variable | VRC13e | US20150044137 SEQ ID NO: 9 | 28072 |
| | region | | | |
| HIV1089 | Heavy chain variable | VRC13f | US20150044137 SEQ ID NO: 10 | 28073 |
| | region | | | |
| HIV1090 | Heavy chain variable | VRC13g | US20150044137 SEQ ID NO: 11 | 28074 |
| | region | | | |
| HIV1091 | Heavy chain variable | VRC13h | US20150044137 SEQ ID NO: 12 | 28075 |
| | region | | | |
| HIV1092 | Heavy chain variable | VRC15 | US20150044137 SEQ ID NO: 16 | 28076 |
| | region | | | |
| HIV1093 | Heavy chain variable | | US20150004190 SEQ ID NO: 56 | 28077 |
| | region | | | |
| HIV1094 | Heavy chain variable | P7 | NCBI Accession # AAB41043.1 (136aa) | 28078 |
| | region, partial | | | |
| HIV1095 | Heavy Chain, Fab | Ch04 | McLellan, J. S. et al., Structure of HIV-1 gp120 | 28079 |
| | | | V1 V2 domain with broadly neutralizing | |
| | | | antibody PG9; Nature 480 (7377), 336-343 | |
| | | | (2011), NCBI Accession # 3TCL_A (237aa) | |
| HIV1096 | Heavy Chain, Fab | N5-i5 | Acharya, P., et al., Structural Definition of an | 28080 |
| | | | Antibody-Dependent Cellular Cytotoxicity | |
| | | | Response Implicated in Reduced Risk for HIV- | |
| | | | 1 infection; J. Virol. 88 (21), 12895-12906 | |
| | | | (2014), NCBI Accession # 4H8W_H (226aa) | |
| HIV1097 | Heavy Chain, Fab | N60-i3 | Gohain, N., et al., Cocrystal Structures of | 28081 |
| | | | Antibody N60-i3 and Antibody JR4 in | |
| | | | Complex with gp120 Define More Cluster A | |
| | | | Epitopes Involved in Effective Antibody- | |
| | | | Dependent Effector Function against HIV-1; J. | |
| | | | Virol. 89 (17), 8840-8854 (2015), NCBI | |
| | | | Accession # 4RFO_H (229aa) | |
| HIV1098 | Heavy Chain, Ig | Nih45-46 Fab | Diskin, R., et al., Science 334 (6060), 1289- | 28082 |
| | Gamma-1 Chain C | | 1293 (2011), NCBI Accession # 3U7Y_H | |
| | Region | | (229aa) | |
| HIV1099 | Heavy Chain, Ig | Pgt127 | Pejchal, R., et al., Science 334 (6059), 1097- | 28083 |
| | Gamma-1 Chain C | | 1103 (2011), NCBI Accession # | |
| | Region | | 3TWC_H(239aa) | |
| HIV1100 | Heavy Chain, Ig | Pgt128 | Pejchal, R., et al., Science 334 (6059), 1097- | 28084 |
| | Gamma-1 Chain C | | 1103 (2011), NCBI Accession # | |
| | Region | | 3TV3_H(239aa) | |
| HIV1101 | HIV, heavy chain | Suvizumab | | 28085 |
| HIV1102 | HIV1 gp120 | HIV1 gp120 | WO2001000678 SEQ ID NO: 43 | 28086 |
| | antibody, heavy chain | antibody | | |
| HIV1103 | HIV1 gp120 | HIV1 gp120 | WO2001000678 SEQ ID NO: 44 | 28087 |
| | antibody, heavy chain | antibody | | |
| HIV1104 | HIV1 gp120 | HIV1 gp120 | WO2001000678 SEQ ID NO: 45 | 28088 |
| | antibody, heavy chain | antibody | | |
| HIV1105 | HIV1 gp120 | HIV1 gp120 | WO2001000678 SEQ ID NO: 46 | 28089 |
| | antibody, heavy chain | antibody | | |
| HIV1106 | HIV1 gp120 | HIV1 gp120 | WO2001000678 SEQ ID NO: 47 | 28090 |
| | antibody, heavy chain | antibody | | |
| HIV1107 | HIV1 gp120 | HIV1 gp120 | WO2001000678 SEQ ID NO: 48 | 28091 |
| | antibody, heavy chain | antibody | | |
| HIV1108 | HIV1 gp120 | HIV1 gp120 | WO2001000678 SEQ ID NO: 49 | 28092 |
| | antibody, heavy chain | antibody | | |
| HIV1109 | HIV1 gp120 | HIV1 gp120 | WO2001000678 SEQ ID NO: 57 | 28093 |
| | antibody, heavy chain | antibody | | |
| HIV1110 | HIV1 gp120 | HIV1 gp120 | WO2001000678 SEQ ID NO: 58 | 28094 |
| | antibody, heavy chain | antibody | | |
| HIV1111 | HIV1 gp120 | HIV1 gp120 | WO2001000678 SEQ ID NO: 59 | 28095 |
| | antibody, heavy chain | antibody | | |
| HIV1112 | HIV1 gp120 | HIV1 gp120 | WO2001000678 SEQ ID NO: 60 | 28096 |
| | antibody, heavy chain | antibody | | |
| HIV1113 | HIV1 gp120 | HIV1 gp120 | WO2001000678 SEQ ID NO: 61 | 28097 |
| | antibody, heavy chain | antibody | | |
| HIV1114 | HIV1 gp120 | HIV1 gp120 | WO2001000678 SEQ ID NO: 62 | 28098 |
| | antibody, heavy chain | antibody | | |
| HIV1115 | HIV1 gp120 | HIV1 gp120 | WO2001000678 SEQ ID NO: 63 | 28099 |
| | antibody, heavy chain | antibody | | |
| HIV1116 | HIV1 gp120 | HIV1 gp120 | WO2001000678 SEQ ID NO: 64 | 28100 |
| | antibody, heavy chain | antibody | | |
| HIV1117 | HIV1 gp120 | HIV1 gp120 | WO2001000678 SEQ ID NO: 73 | 28101 |
| | antibody, heavy chain | antibody | | |
| HIV1118 | HIV1 gp120 | HIV1 gp120 | WO2001000678 SEQ ID NO: 74 | 28102 |
| | antibody, heavy chain | antibody | | |
| HIV1119 | HIV1 gp120 | HIV1 gp120 | WO2001000678 SEQ ID NO: 75 | 28103 |
| | antibody, heavy chain | antibody | | |
| HIV1120 | HIV1 gp120 | HIV1 gp120 | WO2001000678 SEQ ID NO: 76 | 28104 |
| | antibody, heavy chain | antibody | | |
| HIV1121 | HIV1 gp120 | HIV1 gp120 | WO2001000678 SEQ ID NO: 77 | 28105 |
| | antibody, heavy chain | antibody | | |
| HIV1122 | HIV1 gp120 | HIV1 gp120 | WO2001000678 SEQ ID NO: 78 | 28106 |
| | antibody, heavy chain | antibody | | |
| HIV1123 | HIV1 gp120 | HIV1 gp120 | WO2001000678 SEQ ID NO: 50 | 28107 |
| | antibody, light chain | antibody | | |
| HIV1124 | HIV1 gp120 | HIV1 gp120 | WO2001000678 SEQ ID NO: 51 | 28108 |
| | antibody, light chain | antibody | | |
| HIV1125 | HIV1 gp120 | HIV1 gp120 | WO2001000678 SEQ ID NO: 52 | 28109 |
| | antibody, light chain | antibody | | |
| HIV1126 | HIV1 gp120 | HIV1 gp120 | WO2001000678 SEQ ID NO: 53 | 28110 |
| | antibody, light chain | antibody | | |
| HIV1127 | HIV1 gp120 | HIV1 gp120 | WO2001000678 SEQ ID NO: 54 | 28111 |
| | antibody, light chain | antibody | | |
| HIV1128 | HIV1 gp120 | HIV1 gp120 | WO2001000678 SEQ ID NO: 55 | 28112 |
| | antibody, light chain | antibody | | |
| HIV1129 | HIV1 gp120 | HIV1 gp120 | WO2001000678 SEQ ID NO: 56 | 28113 |
| | antibody, light chain | antibody | | |
| HIV1130 | HIV1 gp120 | HIV1 gp120 | WO2001000678 SEQ ID NO: 65 | 28114 |
| | antibody, light chain | antibody | | |
| HIV1131 | HIV1 gp120 | HIV1 gp120 | WO2001000678 SEQ ID NO: 66 | 28115 |
| | antibody, light chain | antibody | | |
| HIV1132 | HIV1 gp120 | HIV1 gp120 | WO2001000678 SEQ ID NO: 67 | 28116 |
| | antibody, light chain | antibody | | |
| HIV1133 | HIV1 gp120 | HIV1 gp120 | WO2001000678 SEQ ID NO: 68 | 28117 |
| | antibody, light chain | antibody | | |
| HIV1134 | HIV1 gp120 | HIV1 gp120 | WO2001000678 SEQ ID NO: 69 | 28118 |
| | antibody, light chain | antibody | | |
| HIV1135 | HIV1 gp120 | HIV1 gp120 | WO2001000678 SEQ ID NO: 70 | 28119 |
| | antibody, light chain | antibody | | |
| HIV1136 | HIV1 gp120 | HIV1 gp120 | WO2001000678 SEQ ID NO: 71 | 28120 |
| | antibody, light chain | antibody | | |
| HIV1137 | HIV1 gp120 | HIV1 gp120 | WO2001000678 SEQ ID NO: 72 | 28121 |
| | antibody, light chain | antibody | | |
| HIV1138 | HIV1 gp120 | HIV1 gp120 | WO2001000678 SEQ ID NO: 79 | 28122 |
| | antibody, light chain | antibody | | |
| HIV1139 | HIV1 gp120 | HIV1 gp120 | WO2001000678 SEQ ID NO: 80 | 28123 |
| | antibody, light chain | antibody | | |
| HIV1140 | HIV1 gp120 | HIV1 gp120 | WO2001000678 SEQ ID NO: 81 | 28124 |
| | antibody, light chain | antibody | | |
| HIV1141 | HIV1 gp120 | HIV1 gp120 | WO2001000678 SEQ ID NO: 82 | 28125 |
| | antibody, light chain | antibody | | |
| HIV1142 | HIV1 gp120 | HIV1 gp120 | WO2001000678 SEQ ID NO: 83 | 28126 |
| | antibody, light chain | antibody | | |
| HIV1143 | Kappa light chain | 1460_G14 | U.S. Pat. No. 9,051,362 SEQ ID NO: 22 | 28127 |
| HIV1144 | Kappa light chain | 1456_P20 | U.S. Pat. No. 9,051,362 SEQ ID NO: 34 | 28128 |
| | variable region | | | |
| HIV1145 | Kappa light chain | 1460_G14 | U.S. Pat. No. 9,051,362 SEQ ID NO: 36 | 28129 |
| | variable region | | | |
| HIV1146 | Kappa light chain | 1456_P20 | U.S. Pat. No. 9,051,362 SEQ ID NO: 18 | 28130 |
| HIV1147 | Lambda light chain | 1456_A12 | U.S. Pat. No. 9,051,362 SEQ ID NO: 50 | 28131 |
| HIV1148 | Lambda light chain | 1469 M23 | U.S. Pat. No. 9,051,362 SEQ ID NO: 142 | 28132 |
| HIV1149 | Lambda light chain | 1489_I13 | U.S. Pat. No. 9,051,362 SEQ ID NO: 14 | 28133 |
| HIV1150 | Lambda light chain | 1495_C14 | U.S. Pat. No. 9,051,362 SEQ ID NO: 26 | 28134 |
| HIV1151 | Lambda light chain | 1489_I13 | U.S. Pat. No. 9,051,362 SEQ ID NO: 32 | 28135 |
| | variable region | | | |
| HIV1152 | Lambda light chain | 1495_C14 | U.S. Pat. No. 9,051,362 SEQ ID NO: 38 | 28136 |
| | variable region | | | |
| HIV1153 | Lambda light chain | 1496_C09 | U.S. Pat. No. 9,051,362 SEQ ID NO: 40 | 28137 |
| | variable region | | | |
| HIV1154 | Lambda light chain | 1456_A12 | U.S. Pat. No. 9,051,362 SEQ ID NO: 51 | 28138 |
| | variable region | | | |
| HIV1155 | Lambda light chain | 1503_H05 | U.S. Pat. No. 9,051,362 SEQ ID NO: 56 | 28139 |
| | variable region | | | |
| HIV1156 | Lambda light chain | 1496_C09 | U.S. Pat. No. 9,051,362 SEQ ID NO: 30 | 28140 |
| HIV1157 | Light chain | 2424 | Kumar, R., et al., Functional and Structural | 28141 |
| | | | Characterization of Human V3-Specific | |
| | | | Monoclonal Antibody 2424 with Neutralizing | |
| | | | Activity against HIV-1 JRFL; J. Virol. 89 (17), | |
| | | | 9090-9102 (2015), NCBI Accession # | |
| | | | 4XML_L(215aa) | |
| HIV1158 | Light chain | 8062 | Gustchina, E., PLoS ONE 8 (11), E78187 | 28142 |
| | | | (2013), NCBI Accession # 4KHX_L(213aa) | |
| HIV1159 | Light chain | 1.00E+09 | US20140348785 SEQ ID NO: 2 | 28143 |
| HIV1160 | Light Chain | 10e8 | Huang J et al., Nature 491 (7424), 406-412 | 28144 |
| | | (monoclonal) | (2012), NCBI Accession # 4G6F_D (215aa) | |
| HIV1161 | Light chain | 12a12kc | US20140328862 SEQ ID NO: 453 | 28145 |
| HIV1162 | Light chain | 12a13kc | US20140328862 SEQ ID NO: 454 | 28146 |
| HIV1163 | Light chain | 12a16kc | US20140328862 SEQ ID NO: 455 | 28147 |
| HIV1164 | Light chain | 12a1kc | US20140328862 SEQ ID NO: 456 | 28148 |
| HIV1165 | Light chain | 12a20kc | US20140328862 SEQ ID NO: 457 | 28149 |
| HIV1166 | Light chain | 12a21 | NCBI Accession # 4JPW_L (210aa) | 28150 |
| HIV1167 | Light chain | 12a21kc | US20140328862 SEQ ID NO: 458 | 28151 |
| HIV1168 | Light chain | 12a22kc | US20140328862 SEQ ID NO: 459 | 28152 |
| HIV1169 | Light chain | 12a23kc | US20140328862 SEQ ID NO: 460 | 28153 |
| HIV1170 | Light chain | 12a27kc | US20140328862 SEQ ID NO: 461 | 28154 |
| HIV1171 | Light chain | 12a46kc | US20140328862 SEQ ID NO: 462 | 28155 |
| HIV1172 | Light chain | 12a55kc | US20140328862 SEQ ID NO: 463 | 28156 |
| HIV1173 | Light chain | 12a56kc | US20140328862 SEQ ID NO: 464 | 28157 |
| HIV1174 | Light chain | 12a6kc | US20140328862 SEQ ID NO: 465 | 28158 |
| HIV1175 | Light chain | 12a7kc | US20140328862 SEQ ID NO: 466 | 28159 |
| HIV1176 | Light chain | 17b | Kwong, P. D., et al., structure of an HIV gp120 | 28160 |
| | | | envelope glycoprotein in complex with the CD4 | |
| | | | receptor and a neutralizing human antibody; | |
| | | | Nature 393 (6686). 648-659 (1998), NCBI | |
| | | | Accession # 1G9M_L(214aa) | |
| HIV1177 | Light chain | 1b2530 | Zhou T et al., Structural Repertoire of HIV-1- | 28161 |
| | | | Neutralizing Antibodies Targeting the CD4 | |
| | | | Supersite in 14 Donors; Cell 161 (6), 1280- | |
| | | | 1292 (2015), NCBI Accession # 4YFL_L | |
| | | | (215aa) | |
| HIV1178 | Light chain | 1F7 | U.S. Pat. No. 6,057,421A FIG. 8 | 28162 |
| HIV1179 | Light chain | 1NC9 | WO2012154312 SEQ ID NO: 2472 | 28163 |
| HIV1180 | Light chain | 2.2C | Acharya, P., et al., Structural Definition of an | 28164 |
| | | | Antibody-Dependent Cellular Cytotoxicity | |
| | | | Response Implicated in Reduced Risk for HIV- | |
| | | | 1 Infection; J. Virol. 88 (21), 12895-12906 | |
| | | | (2014), NCBI Accession # 4R4N_L (210aa) | |
| HIV1181 | Light chain | 2F5 | U.S. Pat. No. 8,637,036B2 SEQ ID NO: 10 | 28165 |
| HIV1182 | Light chain | 3040LC | WO2015117008 SEQ ID NO: 29 | 28166 |
| HIV1183 | Light chain | 3044LC | WO2015117008 SEQ ID NO: 32 | 28167 |
| HIV1184 | Light chain | 3430LC | WO2015117008 SEQ ID NO: 30 | 28168 |
| HIV1185 | Light chain | 3484LC | WO2015117008 SEQ ID NO: 31 | 28169 |
| HIV1186 | Light chain | 3630LC | WO2015117008 SEQ ID NO: 33 | 28170 |
| HIV1187 | Light chain | 3A124KC | US20140328862 SEQ ID NO: 506 | 28171 |
| HIV1188 | Light chain | 3A125KC | US20140328862 SEQ ID NO: 507 | 28172 |
| HIV1189 | Light chain | 3A140LC | US20140328862 SEQ ID NO: 508 | 28173 |
| HIV1190 | Light chain | 3A144KC | US20140328862 SEQ ID NO: 509 | 28174 |
| HIV1191 | Light chain | 3A160KC | US20140328862 SEQ ID NO: 510 | 28175 |
| HIV1192 | Light chain | 3A18KC | US20140328862 SEQ ID NO: 511 | 28176 |
| HIV1193 | Light chain | 3A204KC | US20140328862 SEQ ID NO: 512 | 28177 |
| HIV1194 | Light chain | 3A228KC | US20140328862 SEQ ID NO: 513 | 28178 |
| HIV1195 | Light chain | 3A233LC | US20140328862 SEQ ID NO: 514 | 28179 |
| HIV1196 | Light chain | 3A244LC | US20140328862 SEQ ID NO: 515 | 28180 |
| HIV1197 | Light chain | 3A255LC | US20140328862 SEQ ID NO: 516 | 28181 |
| HIV1198 | Light chain | 3A296KC | US20140328862 SEQ ID NO: 517 | 28182 |
| HIV1199 | Light chain | 3A334LC | US20140328862 SEQ ID NO: 518 | 28183 |
| HIV1200 | Light chain | 3A366KC | US20140328862 SEQ ID NO: 519 | 28184 |
| HIV1201 | Light chain | 3A384KC | US20140328862 SEQ ID NO: 520 | 28185 |
| HIV1202 | Light chain | 3A419KC | US20140328862 SEQ ID NO: 521 | 28186 |
| HIV1203 | Light chain | 3a426kc | US20140328862 SEQ ID NO: 535 | 28187 |
| HIV1204 | Light chain | 3A461KC | US20140328862 SEQ ID NO: 522 | 28188 |
| HIV1205 | Light chain | 3A474KC | US20140328862 SEQ ID NO: 523 | 28189 |
| HIV1206 | Light chain | 3a515kc | US20140328862 SEQ ID NO: 536 | 28190 |
| HIV1207 | Light chain | 3A518KC | US20140328862 SEQ ID NO: 524 | 28191 |
| HIV1208 | Light chain | 3A539LC | US20140328862 SEQ ID NO: 525 | 28192 |
| HIV1209 | Light chain | 3A576LC | US20140328862 SEQ ID NO: 526 | 28193 |
| HIV1210 | Light chain | 3A613LC | US20140328862 SEQ ID NO: 527 | 28194 |
| HIV1211 | Light chain | 3A64KC | US20140328862 SEQ ID NO: 528 | 28195 |
| HIV1212 | Light chain | 3A650KC | US20140328862 SEQ ID NO: 529 | 28196 |
| HIV1213 | Light chain | 3A67KC | US20140328862 SEQ ID NO: 530 | 28197 |
| HIV1214 | Light chain | 3A779KC | US20140328862 SEQ ID NO: 531 | 28198 |
| HIV1215 | Light chain | 3A816KC | US20140328862 SEQ ID NO: 532 | 28199 |
| HIV1216 | Light chain | 3A869KC | US20140328862 SEQ ID NO: 533 | 28200 |
| HIV1217 | Light chain | 3A93LC | US20140328862 SEQ ID NO: 534 | 28201 |
| HIV1218 | Light chain | 3anc3kc | US20140328862 SEQ ID NO: 547 | 28202 |
| HIV1219 | Light chain | 3b106kc | US20140328862 SEQ ID NO: 548 | 28203 |
| HIV1220 | Light chain | 3b129kc | US20140328862 SEQ ID NO: 537 | 28204 |
| HIV1221 | Light chain | 3b16kc | US20140328862 SEQ ID NO: 549 | 28205 |
| HIV1222 | Light chain | 3b171lc | US20140328862 SEQ ID NO: 538 | 28206 |
| HIV1223 | Light chain | 3b180kc | US20140328862 SEQ ID NO: 550 | 28207 |
| HIV1224 | Light chain | 3b183kc | US20140328862 SEQ ID NO: 551 | 28208 |
| HIV1225 | Light chain | 3b191kc | US20140328862 SEQ ID NO: 552 | 28209 |
| HIV1226 | Light chain | 3b21kc | US20140328862 SEQ ID NO: 553 | 28210 |
| HIV1227 | Light chain | 3b27kc | US20140328862 SEQ ID NO: 539 | 28211 |
| HIV1228 | Light chain | 3b41kc | US20140328862 SEQ ID NO: 540 | 28212 |
| HIV1229 | Light chain | 3b46kc | US20140328862 SEQ ID NO: 542 | 28213 |
| HIV1230 | Light chain | 3b57lc | US20140328862 SEQ ID NO: 543 | 28214 |
| HIV1231 | Light chain | 3b5kc | US20140328862 SEQ ID NO: 541 | 28215 |
| HIV1232 | Light chain | 3b8kc | US20140328862 SEQ ID NO: 544 | 28216 |
| HIV1233 | Light chain | 3bnc102kc | US20140328862 SEQ ID NO: 554 | 28217 |
| HIV1234 | Light chain | 3bnc104kc | US20140328862 SEQ ID NO: 555 | 28218 |
| HIV1235 | Light chain | 3bnc105kc | US20140328862 SEQ ID NO: 556 | 28219 |
| HIV1236 | Light chain | 3bnc107kc | US20140328862 SEQ ID NO: 557 | 28220 |
| HIV1237 | Light chain | 3bnc108kc | US20140328862 SEQ ID NO: 558 | 28221 |
| HIV1238 | Light chain | 3bnc117 | Zhou T et al., Immunity 39 (2), 245-258 (2013), | 28222 |
| | | | NCBI Accession # 4LSV_L(206aa) | |
| HIV1239 | Light chain | 3bnc117kc | US20140328862 SEQ ID NO: 559 | 28223 |
| HIV1240 | Light chain | 3bnc134kc | US20140328862 SEQ ID NO: 560 | 28224 |
| HIV1241 | Light chain | 3bnc142kc | US20140328862 SEQ ID NO: 561 | 28225 |
| HIV1242 | Light chain | 3bnc151kc | US20140328862 SEQ ID NO: 562 | 28226 |
| HIV1243 | Light chain | 3bnc153kc | US20140328862 SEQ ID NO: 563 | 28227 |
| HIV1244 | Light chain | 3bnc156kc | US20140328862 SEQ ID NO: 564 | 28228 |
| HIV1245 | Light chain | 3bnc158kc | US20140328862 SEQ ID NO: 565 | 28229 |
| HIV1246 | Light chain | 3bnc159kc | US20140328862 SEQ ID NO: 566 | 28230 |
| HIV1247 | Light chain | 3bnc15kc | US20140328862 SEQ ID NO: 567 | 28231 |
| HIV1248 | Light chain | 3bnc176kc | US20140328862 SEQ ID NO: 568 | 28232 |
| HIV1249 | Light chain | 3bnc193kc | US20140328862 SEQ ID NO: 569 | 28233 |
| HIV1250 | Light chain | 3bnc196kc | US20140328862 SEQ ID NO: 570 | 28234 |
| HIV1251 | Light chain | 3bnc31kc | US20140328862 SEQ ID NO: 571 | 28235 |
| HIV1252 | Light chain | 3bnc42kc | US20140328862 SEQ ID NO: 572 | 28236 |
| HIV1253 | Light chain | 3bnc53kc | US20140328862 SEQ ID NO: 573 | 28237 |
| HIV1254 | Light chain | 3BNC55KC | US20140328862 SEQ ID NO: 545 | 28238 |
| HIV1255 | Light chain | 3BNC60KC | US20140328862 SEQ ID NO: 546 | 28239 |
| HIV1256 | Light chain | 3bnc62kc | US20140328862 SEQ ID NO: 574 | 28240 |
| HIV1257 | Light chain | 3bnc65kc | US20140328862 SEQ ID NO: 575 | 28241 |
| HIV1258 | Light chain | 3bnc66kc | US20140328862 SEQ ID NO: 576 | 28242 |
| HIV1259 | Light chain | 3bnc75kc | US20140328862 SEQ ID NO: 577 | 28243 |
| HIV1260 | Light chain | 3bnc79kc | US20140328862 SEQ ID NO: 578 | 28244 |
| HIV1261 | Light chain | 3bnc81kc | US20140328862 SEQ ID NO: 579 | 28245 |
| HIV1262 | Light chain | 3bnc84kc | US20140328862 SEQ ID NO: 580 | 28246 |
| HIV1263 | Light chain | 3bnc87kc | US20140328862 SEQ ID NO: 581 | 28247 |
| HIV1264 | Light chain | 3bnc89kc | US20140328862 SEQ ID NO: 582 | 28248 |
| HIV1265 | Light chain | 3bnc91kc | US20140328862 SEQ ID NO: 583 | 28249 |
| HIV1266 | Light chain | 3bnc95kc | US20140328862 SEQ ID NO: 584 | 28250 |
| HIV1267 | Light chain | 412d | Huang et al., Science 317 (5846), 1930-1934 | 28251 |
| | | | (2007), NCBI Accession # 2QAD_G (214aa) | |
| HIV1268 | Light Chain | 44-vrc13.01 | Zhon T et al., Structural Repertoire of HIV-1- | 28252 |
| | | | Neutralizing Antibodies Targeting the CD4 | |
| | | | Supersite in 14 Donors; Cell 161 (6), 1280- | |
| | | | 1292 (2015), NCBI Accession # 4YDJ_B | |
| | | | (206aa) | |
| HIV1269 | Light chain | 45-46m2 | Diskin, R., et al., Restricting HIV-1 pathways | 28253 |
| | | | for escape using rationally designed anti-HIV-1 | |
| | | | antibodies; J. Exp. Med. 210 (6), 1235-1249 | |
| | | | (2013), NCBI Accession # 4JKP_L (210aa) | |
| HIV1270 | Light chain | 4835_F12 | US20140205612 SEQ ID NO: 413 | 28254 |
| | | (PGT-124) | | |
| HIV1271 | Light chain | 4838_L06 | US20140205612 SEQ ID NO: 148 | 28255 |
| | | (PGT-121) | | |
| HIV1272 | Light chain | 4858_P08 | US20140205612 SEQ ID NO: 176 | 28256 |
| | | (PGT-123) | | |
| HIV1273 | Light chain | 4869-K15 | US20140205612 SEQ ID NO: 428 | 28257 |
| | | (PGT-133) | | |
| HIV1274 | Light chain | 4873_E03 | US20140205612 SEQ ID NO: 147 | 28258 |
| | | (PGT-121) | | |
| HIV1275 | Light chain | 4876_M06 | US20140205612 SEQ ID NO: 439 | 28259 |
| | | (PGT-134) | | |
| HIV1276 | Light chain | 4877_D15 | US20140205612 SEQ ID NO: 160 | 28260 |
| | | (PGT-122) | | |
| HIV1277 | Light chain | 4964_G22 | US20140205612 SEQ ID NO: 284 | 28261 |
| | | (PGT-141), | | |
| | | 4993_K13 | | |
| | | (PGT-141), | | |
| | | 4995_E20 | | |
| | | (PGT-142) | | |
| HIV1278 | Light chain | 4970_K22 | US20140205612 SEQ ID NO: 312 | 28262 |
| | | (PGT-144) | | |
| HIV1279 | Light chain | 4980_N08 | US20140205612 SEQ ID NO: 301 | 28263 |
| | | (PGT-143) | | |
| HIV1280 | Light chain | 4995_P16 | US20140205612 SEQ ID NO: 385 | 28264 |
| | | (PGT-145) | | |
| HIV1281 | Light chain | 4e10 Fv | Finton, K. A., et al., PLoS Pathol. 9 (9), | 28265 |
| | | | E1003639 (2013), NCBI Accession # 4LLV_B | |
| | | | (112aa) | |
| HIV1282 | Light chain | 5114_A19 | US20140205612 SEQ ID NO: 392 | 28266 |
| | | (PGT-128) | | |
| HIV1283 | Light chain | 5120_N10 | US20140205612 SEQ ID NO: 469 | 28267 |
| | | (PGT-139) | | |
| HIV1284 | Light chain | 5131_A17 | US20140205612 SEQ ID NO: 488 | 28268 |
| | | (PGT-132) | | |
| HIV1285 | Light chain | 5136_H01 | US20140205612 SEQ ID NO: 355 | 28269 |
| | | (PGT-131) | | |
| HIV1286 | Light chain | 5138_G07 | US20140205612 SEQ ID NO: 483 | 28270 |
| | | (PGT-138) | | |
| HIV1287 | Light chain | 5141_B17 | US20140205612 SEQ ID NO: 208 | 28271 |
| | | (PGT-126) | | |
| HIV1288 | Light chain | 5145_B14 | US20140205612 SEQ ID NO: 329 | 28272 |
| | | (PGT-127) | | |
| HIV1289 | Light chain | 5147_N06 | US20140205612 SEQ ID NO: 244 | 28273 |
| | | (PGT-130) | | |
| HIV1290 | Light chain | 5329_C19 | US20140205612 SEQ ID NO: 257 | 28274 |
| | | (PGT-136), | | |
| | | 5366_P21 | | |
| | | (PGT-136) | | |
| HIV1291 | Light chain | 5343_B08 | US20140205612 SEQ ID NO: 240 | 28275 |
| | | (PGT-135), | | |
| | | 5344_E16 | | |
| | | (PGT-135) | | |
| HIV1292 | Light chain | 5345_I01 | US20140205612 SEQ ID NO: 396 | 28276 |
| | | (PGT-137) | | |
| HIV1293 | Light chain | 6808_B09 | US20140205612 SEQ ID NO: 553 | 28277 |
| | | (PGT-156) | | |
| HIV1294 | Light chain | 6831_A21 | US20140205612 SEQ ID NO: 482 | 28278 |
| | | (PGT-151) | | |
| HIV1295 | Light chain | 6843_G20 | US20140205612 SEQ ID NO: 524 | 28279 |
| | | (PGT-154) | | |
| HIV1296 | Light chain | 6881_N05 | US20140205612 SEQ ID NO: 578 | 28280 |
| | | (PGT-158). | | |
| HIV1297 | Light chain | 6889_I17 | US20140205612 SEQ ID NO: 496 | 28281 |
| | | (PGT-152) | | |
| HIV1298 | Light chain | 6891_F06 | US20140205612 SEQ ID NO: 510 | 28282 |
| | | (PGT-153) | | |
| HIV1299 | Light chain | 6892_C23 | US20140205612 SEQ ID NO: 565 | 28283 |
| | | (PGT-157) | | |
| HIV1300 | Light chain | 6892_D19 | US20140205612 SEQ ID NO: 539 | 28284 |
| | | (PGT-155) | | |
| HIV1301 | Light chain | 7H6 | US20140348785 SEQ ID NO: 4 | 28285 |
| HIV1302 | Light chain | 7N16 | US20140348785 SEQ ID NO: 6 | 28286 |
| HIV1303 | Light chain | 8anc131 | Zhou T et al. Structural Repertoire of HIV-1- | 28287 |
| | | | Neutralizing Antibodies Targeting the CD4 | |
| | | | Supersite in 14 Donors; Cell 161 (6), 1280- | |
| | | | 1292 (2015), NCBI Accession # 4RWY_L | |
| | | | (213aa) | |
| HIV1304 | Light chain | 8ANC131KC | US20140328862 SEQ ID NO: 440 | 28288 |
| HIV1305 | Light chain | 8anc134 | Zhou T et al, Structural Repertoire of HIV-1- | 28289 |
| | | | Neutralizing Antibodies Targeting the CD4 | |
| | | | Supersite in 14 Donors; Cell 161 (6), 1280- | |
| | | | 1292 (2015), NCBI Accession # 4RX4_L | |
| | | | (213aa) | |
| HIV1306 | Light chain | 8ANC134KC | US20140328862 SEQ ID NO: 441 | 28290 |
| HIV1307 | Light chain | 8ANC13KC | US20140328862 SEQ ID NO: 442 | 28291 |
| HIV1308 | Light chain | 8ANC14KC | US20140328862 SEQ ID NO: 448 | 28292 |
| HIV1309 | Light chain | 8ANC16KC | US20140328862 SEQ ID NO: 449 | 28293 |
| HIV1310 | Light chain | 8anc182kc | US20140328862 SEQ ID NO: 446 | 28294 |
| HIV1311 | Light chain | 8anc192kc | US20140328862 SEQ ID NO: 447 | 28295 |
| HIV1312 | Light chain | 8ANC195KC | US20140328862 SEQ ID NO: 450 | 28296 |
| HIV1313 | Light chain | 8ANC24KC | US20140328862 SEQ ID NO: 451 | 28297 |
| HIV1314 | Light chain | 8ANC45KC | US20140328862 SEQ ID NO: 443 | 28298 |
| HIV1315 | Light chain | 8ANC50KC | US20140328862 SEQ ID NO: 444 | 28299 |
| HIV1316 | Light chain | 8ANC5KC | US20140328862 SEQ ID NO: 452 | 28300 |
| HIV1317 | Light chain | 8ANC88KC | US20140328862 SEQ ID NO: 445 | 28301 |
| HIV1318 | Light chain | Anti-HcG | Fotinou C. et al “Structure of an Fab fragment | 28302 |
| | | | against a C-terminal peptide of hCG at 2.0 A | |
| | | | resolution” J. Biol. Chem. 273 (35), 22515- | |
| | | | 22518 (1998); NCBI Accession # 1SBS_L | |
| HIV1319 | Light chain | B12 | Zhou T et al., Structural definition of a | 28303 |
| | | | conserved neutralization epitope on HIV-1 | |
| | | | gp120; Nature 445 (7129), 732-737 (2007), | |
| | | | NCBI Accession # 2NY7_L (215aa) | |
| HIV1320 | Light Chain | C38-vrc16.01 | Zhou T et al., Structural Repertoire of HIV-1- | 28304 |
| | | | Neutralizing Antibodies Targeting the CD4 | |
| | | | Supersite in 14 Donors; Cell 161 (6), 1280- | |
| | | | 1292 (2015), NCBI Accession # 4YDK_L | |
| | | | (214aa) | |
| HIV1321 | Light chain | C38-vrc18.02 | Zhou T et al., Structural Repertoire of HIV-1- | 28305 |
| | | | Neutralizing Antibodies Targeting the CD4 | |
| | | | Supersite in 14 Donors; Cell 161 (6), 1280- | |
| | | | 1292 (2015), NCBI Accession # 4YDL_L | |
| | | | (211aa) | |
| HIV1322 | Light chain | CAP256- | WO2015128846 SEQ ID NO: 14 | 28306 |
| | | VRC26.01 | | |
| HIV1323 | Light chain | CAP256- | WO2015128846 SEQ ID NO: 18 | 28307 |
| | | VRC26.02 | | |
| HIV1324 | Light chain | CAP256- | WO2015128846 SEQ ID NO: 22 | 28308 |
| | | VRC26.03 | | |
| HIV1325 | Light chain | CAP256- | WO2015128846 SEQ ID NO: 26 | 28309 |
| | | VRC26.04 | | |
| HIV1326 | Light chain | CAP256- | WO2015128846 SEQ ID NO: 30 | 28310 |
| | | VRC26.05 | | |
| HIV1327 | Light chain | CAP256- | WO2015128846 SEQ ID NO: 34 | 28311 |
| | | VRC26.06 | | |
| HIV1328 | Light chain | CAP256- | WO2015128846 SEQ ID NO: 38 | 28312 |
| | | VRC26.07 | | |
| HIV1329 | Light chain | CAP256- | WO2015128846 SEQ ID NO: 42 | 28313 |
| | | VRC26.08 | | |
| HIV1330 | Light chain | CAP256- | WO2015128846 SEQ ID NO: 46 | 28314 |
| | | VRC26.09 | | |
| HIV1331 | Light chain | CAP256- | WO2015128846 SEQ ID NO: 50 | 28315 |
| | | VRC26.10 | | |
| HIV1332 | Light chain | CAP256- | WO2015128846 SEQ ID NO: 54 | 28316 |
| | | VRC26.11 | | |
| HIV1333 | Light chain | CAP256- | WO2015128846 SEQ ID NO: 58 | 28317 |
| | | VRC26.12 | | |
| HIV1334 | Light chain | CAP256- | WO2015128846 SEQ ID NO: 171 | 28318 |
| | | VRC26.25 | | |
| HIV1335 | Light chain | CAP256- | WO2015128846 SEQ ID NO: 179 | 28319 |
| | | VRC26.26 | | |
| HIV1336 | Light chain | CAP256- | WO2015128846 SEQ ID NO: 187 | 28320 |
| | | VRC26.27 | | |
| HIV1337 | Light chain | CAP256- | WO2015128846 SEQ ID NO: 6 | 28321 |
| | | VRC26-I1 | | |
| HIV1338 | Light chain | CAP256- | WO2015128846 SEQ ID NO: 10 | 28322 |
| | | VRC26-I2 | | |
| HIV1339 | Light chain | CAP256- | WO2015128846 SEQ ID NO: 2 | 28323 |
| | | VRC26- | | |
| | | UCA. | | |
| HIV1340 | Light chain | construct | WO2015013390 SEQ ID NO: 5 | 28324 |
| | | #2816, #2861 | | |
| HIV1341 | Light chain | construct | WO2015013390 SEQ ID NO: 6 | 28325 |
| | | #2817, #2860 | | |
| HIV1342 | Light chain | construct | WO2015013390 SEQ ID NO: 7 | 28326 |
| | | #2858, | | |
| | | #2859, #2861 | | |
| HIV1343 | Light chain | Fab 2219 | Stanfield, R. L., et al., J. Virol. 80 (12), 6093- | 28327 |
| | | | 6105 (2006), NCBI Accession # 2B0S_L | |
| | | | (215aa) | |
| HIV1344 | Light chain | Fab 2g12 | Doores, K. J., et al., J. Virol. 84 (20), 10690- | 28328 |
| | | | 10699 (2010), NCBI Accession # | |
| | | | 3OAU_L(212a) | |
| HIV1345 | Light chain | Fab 2g12 | Stanfield, R. L. et al., Crystal structure of the | 28329 |
| | | | HIV neutralizing antibody 2G12, in complex | |
| | | | with a bacterial oligosaccharide analog of | |
| | | | mammalian oligomannose; Glycobiology 25 | |
| | | | (4), 412-419 (2015), NCBI Accession # | |
| | | | 4RBP_L (213aa) | |
| HIV1346 | Light chain | Fab F425- | Bell et al., J. Mol. Biol. 375 (4), 969-978 | 28330 |
| | | b4e8 | (2008), NCBI Accession # 2QSC_L (215aa) | |
| HIV1347 | Light chain | G4D | US20130195881 SEQ ID NO: 39 | 28331 |
| HIV1348 | Light chain | G4H | US20130195881 SEQ ID NO: 38 | 28332 |
| HIV1349 | Light chain | gVRC-H5(d74)/ | WO2013090644 SEQ ID NO: 19 | 28333 |
| | | VRC-PG04LC, | | |
| | | gVRCOH12(D74)/ | | |
| | | VRC-PG04LC | | |
| HIV1350 | Light chain | 12 (unbound) | Fera, D. et al., Affinity maturation in an HIV | 28334 |
| | | From Ch103 | broadly neutralizing B-cell lineage through | |
| | | Lineage | reorientation of variable domains; Proc. Natl. | |
| | | | Acad. Sci. U.S.A. 111 (28), 10275-10280 | |
| | | | (2014), NCBI Accession # 4QHN_B (213aa) | |
| HIV1351 | Light chain | IGLV3- | US20140348785 SEQ ID NO: 8 | 28335 |
| | | 19*01 | | |
| HIV1352 | Light chain | k3 | WO2015117008 SEQ ID NO: 19 | 28336 |
| HIV1353 | Light chain | k5 | WO2015117008 SEQ ID NO: 20 | 28337 |
| HIV1354 | Light chain | k53 | WO2015117008 SEQ ID NO: 24 | 28338 |
| HIV1355 | Light chain | k59 | WO2015117008 SEQ ID NO: 21 | 28339 |
| HIV1356 | Light chain | k61 | WO2015117008 SEQ ID NO: 25 | 28340 |
| HIV1357 | Light chain | k62 | WO2015117008 SEQ ID NO: 22 | 28341 |
| HIV1358 | Light chain | k81 | WO2015117008 SEQ ID NO: 28 | 28342 |
| HIV1359 | Light chain | kl 1 | WO2015117008 SEQ ID NO: 26 | 28343 |
| HIV1360 | Light chain | kl8 | WO2015117008 SEQ ID NO: 23 | 28344 |
| HIV1361 | Light chain | kl9 | WO2015117008 SEQ ID NO: 27 | 28345 |
| HIV1362 | Light chain | LSSB2066KC | US20140328862 SEQ ID NO: 501 | 28346 |
| HIV1363 | Light chain | LSSB2080KC | US20140328862 SEQ ID NO: 502 | 28347 |
| HIV1364 | Light chain | LSSB2133KC | US20140328862 SEQ ID NO: 503 | 28348 |
| HIV1365 | Light chain | LSSB2182KC | US20140328862 SEQ ID NO: 504 | 28349 |
| HIV1366 | Light chain | LSSB2339LC | US20140328862 SEQ ID NO: 467 | 28350 |
| HIV1367 | Light chain | LSSB2351LC | US20140328862 SEQ ID NO: 468 | 28351 |
| HIV1368 | Light chain | LSSB2364LC | US20140328862 SEQ ID NO: 469 | 28352 |
| HIV1369 | Light chain | LSSB2367LC | US20140328862 SEQ ID NO: 470 | 28353 |
| HIV1370 | Light chain | LSSB2490LC | US20140328862 SEQ ID NO: 471 | 28354 |
| HIV1371 | Light chain | LSSB2530LC | US20140328862 SEQ ID NO: 472 | 28355 |
| HIV1372 | Light chain | LSSB2554LC | US20140328862 SEQ ID NO: 473 | 28356 |
| HIV1373 | Light chain | LSSB2586LC | US20140328862 SEQ ID NO: 474 | 28357 |
| HIV1374 | Light chain | LSSB2612LC | US20140328862 SEQ ID NO: 475 | 28358 |
| HIV1375 | Light chain | LSSB2640LC | US20140328862 SEQ ID NO: 476 | 28359 |
| HIV1376 | Light chain | LSSB2644LC | US20140328862 SEQ ID NO: 477 | 28360 |
| HIV1377 | Light chain | LSSB2666LC | US20140328862 SEQ ID NO: 478 | 28361 |
| HIV1378 | Light chain | LSSB2680LC | US20140328862 SEQ ID NO: 479 | 28362 |
| HIV1379 | Light chain | LSSB2683LC | US20140328862 SEQ ID NO: 480 | 28363 |
| HIV1380 | Light chain | LSSB331KC | US20140328862 SEQ ID NO: 505 | 28364 |
| HIV1381 | Light chain | LSSB344LC | US20140328862 SEQ ID NO: 481 | 28365 |
| HIV1382 | Light chain | LSSNEC107LC | US20140328862 SEQ ID NO: 482 | 28366 |
| HIV1383 | Light chain | LSSNEC108LC | US20140328862 SEQ ID NO: 483 | 28367 |
| HIV1384 | Light chain | LSSNEC117LC | US20140328862 SEQ ID NO: 484 | 28368 |
| HIV1385 | Light chain | LSSNEC118LC | US20140328862 SEQ ID NO: 485 | 28369 |
| HIV1386 | Light chain | LSSNEC122LC | US20140328862 SEQ ID NO: 486 | 28370 |
| HIV1387 | Light chain | LSSNEC24LC | US20140328862 SEQ ID NO: 487 | 28371 |
| HIV1388 | Light chain | LSSNEC2LC | US20140328862 SEQ ID NO: 488 | 28372 |
| HIV1389 | Light chain | LSSNEC33LC | US20140328862 SEQ ID NO: 489 | 28373 |
| HIV1390 | Light chain | LSSNEC46LC | US20140328862 SEQ ID NO: 490 | 28374 |
| HIV1391 | Light chain | LSSNEC48LC | US20140328862 SEQ ID NO: 491 | 28375 |
| HIV1392 | Light chain | LSSNEC52LC | US20140328862 SEQ ID NO: 492 | 28376 |
| HIV1393 | Light chain | LSSNEC56LC | US20140328862 SEQ ID NO: 493 | 28377 |
| HIV1394 | Light chain | LSSNEC60LC | US20140328862 SEQ ID NO: 494 | 28378 |
| HIV1395 | Light chain | LSSNEC70LC | US20140328862 SEQ ID NO: 495 | 28379 |
| HIV1396 | Light chain | LSSNEC72LC | US20140328862 SEQ ID NO: 496 | 28380 |
| HIV1397 | Light chain | LSSNEC7LC | US20140328862 SEQ ID NO: 497 | 28381 |
| HIV1398 | Light chain | LSSNEC89LC | US20140328862 SEQ ID NO: 498 | 28382 |
| HIV1399 | Light chain | LSSNEC94LC | US20140328862 SEQ ID NO: 499 | 28383 |
| HIV1400 | Light chain | LSSNEC9LC | US20140328862 SEQ ID NO: 500 | 28384 |
| HIV1401 | Light chain | m12-Fd-aa | U.S. Pat. No. 7,803,913B2 SEQ ID NO: 7 | 28385 |
| HIV1402 | Light chain | m14-Fd-aa | U.S. Pat. No. 7,803,913B2 SEQ ID NO: 5 | 28386 |
| HIV1403 | Light chain | m16-Fd-aa | U.S. Pat. No. 7,803,913B2 SEQ ID NO: 8 | 28387 |
| HIV1404 | Light chain | m18 Fd-aa | U.S. Pat. No. 7,803,913B2 SEQ ID NO: 6 | 28388 |
| HIV1405 | Light chain | M66 | Ofek, G., et al., Structural Basis for HIV-1 | 28389 |
| | | | Neutralization by 2F5-Like Antibodies m66 | |
| | | | and m66.6; J. Virol. 88 (5), 2426-2441 (2014), | |
| | | | NCBI Accession # 4NRY_H (235aa) | |
| HIV1406 | Light chain | M66.6 | Ofek, G., et al., Structural Basis for HIV-1 | 28390 |
| | | | Neutralization by 2F5-Like Antibodies m66 | |
| | | | and m66.6; J. Virol. 88 (5), 2426-2441 (2014), | |
| | | | NCBI Accession # 4NRZ_L (213aa) | |
| HIV1407 | Light Chain | Mab 2158 | Spurrier, B., et al., Functional Implications of | 28391 |
| | | | the Binding Mode of a Human Conformation- | |
| | | | Dependent V2 Monoclonal Antibody against | |
| | | | HIV; J. Virol, 88 (8), 4100-4112 (2014), NCBI | |
| | | | Accession # 4OAW_C (214aa) | |
| HIV1408 | Light chain | MV1 | US20130195881 SEQ ID NO: 40 | 28392 |
| HIV1409 | Light chain | Pg16 Fab | Pancera, M., et al., Nat. Struct. Mol. Biol. 20 | 28393 |
| | | | (7), 804-813 (2013), NCBI Accession # | |
| | | | 4DQO_L (216aa) | |
| HIV1410 | Light chain | Pg9 | Willis, J. R., et al., J. Clin. Invest. 125 (6), 2523- | 28394 |
| | | | 2531 (2015), NCBI Accession # 4YAQ_L | |
| | | | (216aa) | |
| HIV1411 | Light chain | Pgt121-Gl | Mouquet H et al., Complex-type N-glycan | 28395 |
| | | Fab | recognition by potent broadly neutralizing HIV | |
| | | | antibodies; Proc Natl Acad Sci USA. 2012 | |
| | | | Nov. 20; 109(47): E3268-77, NCBI Accession # | |
| | | | 4FQQ_A (215aa) | |
| HIV1412 | Light chain | Pgt122 | Julien, J. P., et al., PLoS Pathol. 9 (5), | 28396 |
| | | | E1003342 (2013)”, NCBI Accession # 4JY5_L | |
| | | | (211aa) | |
| HIV1413 | Light chain | Pgt123 | Julien, J. P., et al., PLoS Pathol. 9 (5), | 28397 |
| | | | E1003342 (2013)”, NCBI Accession # 4JY6_A | |
| | | | (211aa) | |
| HIV1414 | Light chain | Pgt124 | Garces, F., et al., Structural Evolution of | 28398 |
| | | | Glycan Recognition by a Family of Potent HIV | |
| | | | Antibodies; Cell 159 (1), 69-79 (2014), NCBI | |
| | | | Accession # 4R26_L (214aa) | |
| HIV1415 | Light chain | Pgt130 | Doores, K. J., et al., J. Virol. 89 (2), 1105-1118 | 28399 |
| | | | (2015), NCBI Accession # 4RNR_B (216aa) | |
| HIV1416 | Light chain | Pgt135 | Grover et al., Science 343 (6171), 656-661 | 28400 |
| | | | (2014), NCBI Accession # 4NZR_L (214aa) | |
| HIV1417 | Light chain | S8, S19, S20 | US20110059015 SEQ ID NO: 2 | 28401 |
| HIV1418 | light chain | Suvizumab | | 28402 |
| HIV1419 | Light Chain | Vrc- Pg04 | Wu, X., et al., Focused evolution of HIV-1 | 28403 |
| | | | neutralizing antibodies revealed by structures | |
| | | | and deep sequencing; Science 333 (6049), | |
| | | | 1593-1602 (2011)”, NCBI Accession # 3SE9_L | |
| | | | (208aa) | |
| HIV1420 | Light chain | VRC01 | U.S. Pat. No. 8,637,036B2 SEQ ID NO: 2 | 28404 |
| HIV1421 | Light chain | VRC01 | US2014 0322163 SEQ ID NO: 53 | 28405 |
| | | E1/12 | | |
| | | deletion | | |
| HIV1422 | Light chain | VRC01 | US2014 0322163 SEQ ID NO: 222 | 28406 |
| | | E1/I2del | | |
| | | F97D | | |
| HIV1423 | Light chain | VRC01 | US2014 0322163 SEQ ID NO: 225 | 28407 |
| | | E1/I2del | | |
| | | F97H | | |
| HIV1424 | Light chain | VRC01 | US2014 0322163 SEQ ID NO: 223 | 28408 |
| | | E1/I2del | | |
| | | F97K | | |
| HIV1425 | Light chain | VRC01 | US2014 0322163 SEQ ID NO: 224 | 28409 |
| | | E1/I2del | | |
| | | F97S | | |
| HIV1426 | Light chain | VRC01 | US2014 0322163 SEQ ID NO: 219 | 28410 |
| | | E1/I2del V3E | | |
| HIV1427 | Light chain | VRC01 | US2014 0322163 SEQ ID NO: 227 | 28411 |
| | | E1/I2del | | |
| | | V3E, F97H | | |
| HIV1428 | Light chain | VRC01 | US2014 0322163 SEQ ID NO: 226 | 28412 |
| | | E1/I2del | | |
| | | V3E, F97S | | |
| HIV1429 | Light chain | VRC01 | US2014 0322163 SEQ ID NO: 220 | 28413 |
| | | E1/I2del V3K | | |
| HIV1430 | Light chain | VRC01 | US2014 0322163 SEQ ID NO: 221 | 28414 |
| | | E1/I2del V3S | | |
| HIV1431 | Light chain | VRC01HC/ | WO2013090644 SEQ ID NO: 31 | 28415 |
| | | VRC03LC | | |
| HIV1432 | Light chain | VRC01hpL02 | US2014 0322163 SEQ ID NO: 50 | 28416 |
| HIV1433 | Light chain | VRC01hpL02 | US2014 0322163 SEQ ID NO: 232 | 28417 |
| | | E1/I2- | | |
| | | deletion, V3S | | |
| HIV1434 | Light chain | VRC01hpL03 | US2014 0322163 SEQ ID NO: 228 | 28418 |
| HIV1435 | Light chain | VRC01hpL04 | US2014 0322163 SEQ ID NO: 229 | 28419 |
| HIV1436 | Light chain | VRC01hpL05 | US2014 0322163 SEQ ID NO: 230 | 28420 |
| HIV1437 | Light chain | VRC01hpL06 | US2014 0322163 SEQ ID NO: 231 | 28421 |
| HIV1438 | Light chain | VRC01LhpL | US2014 0322163 SEQ ID NO: 233 | 28422 |
| | | 03 E1/I2- | | |
| | | deletion, V3S | | |
| HIV1439 | Light chain | VRC01LhpL | US2014 0322163 SEQ ID NO: 237 | 28423 |
| | | 04 E1/I2- | | |
| | | deletion, V3E | | |
| HIV1440 | Light chain | VRC01LhpL | US2014 0322163 SEQ ID NO: 234 | 28424 |
| | | 04 E1/I2- | | |
| | | deletion, V3S | | |
| HIV1441 | Light chain | VRC01LhpL | US2014 0322163 SEQ ID NO: 235 | 28425 |
| | | 05 E1/12 | | |
| | | deletion, V3S | | |
| HIV1442 | Light chain | VRC01LhpL | US2014 0322163 SEQ ID NO: 236 | 28426 |
| | | 06 E1/I2- | | |
| | | deletion, V3S | | |
| HIV1443 | Light chain | VRC02 | U.S. Pat. No. 8,637,036B2 SEQ ID NO: 4 | 28427 |
| HIV1444 | Light chain | VRC03 | U.S. Pat. No. 8,637,036B2 SEQ ID NO: 28 | 28428 |
| HIV1445 | Light chain | VRC03HC- | WO2013090644 SEQ ID NO: 1 | 28429 |
| | | VRC01LC | | |
| HIV1446 | Light chain | Vrc06b | Wu, X., et al., Maturation and Diversity of the | 28430 |
| | | | VRC01-Antibody Lineage over 15 Years of | |
| | | | Chronic HIV-1 Infection; Cell 161 (3), 470-485 | |
| | | | (2015), NCBI Accession # 4XNZ_F (209aa) | |
| HIV1447 | Light chain | Vrc08c | Wu, X., et al., Maturation and Diversity of the | 28431 |
| | | | VRC01-Antibody Lineage over 15 Years of | |
| | | | Chronic HIV-1 Infection; Cell 161 (3), 470-485 | |
| | | | (2015), NCBI Accession # 4XNY_L (211aa) | |
| HIV1448 | Light chain | Vrc23 | Georgiev, I. S., et al., Delineating antibody | 28432 |
| | | | recognition in polyclonal sera from patterns of | |
| | | | HIV-1 isolate neutralization; Science 340 | |
| | | | (6133), 751-756 (2013), NCBI Accession # | |
| | | | 4J6R_L (210aa) | |
| HIV1449 | Light chain | VRC-CH30 | WO2013090644 SEQ ID NO: 21 | 28433 |
| HIV1450 | Light chain | Vrc-ch31 | Zhou T et al., Immunity 39 (2), 245-258 (2013), | 28434 |
| | | | NCBI Accession # 4LSP_L (210aa) | |
| HIV1451 | Light chain | VRC-CH32 | Wu X. et al., “Focused evolution of HIV-1 | 28435 |
| | | | neutralizing antibodies revealed by structures | |
| | | | and deep sequencing” Science 333 (6049), | |
| | | | 1593-1602 (2011), NCBI Accession # | |
| | | | AEM62727 | |
| HIV1452 | Light chain | VRC-CH33 | WO2013090644 SEQ ID NO: 27 | 28436 |
| HIV1453 | Light chain | VRC-CH34 | WO2013090644 SEQ ID NO: 29 | 28437 |
| HIV1454 | Light chain | VRC-PG04 | Wu X. et al,“Focused evolution of HIV-1 | 28438 |
| | | | neutralizing antibodies revealed by structures | |
| | | | and deep sequencing” Science 333 (6049), | |
| | | | 1593-1602 (2011), NCBI Accession # | |
| | | | AEM62754 | |
| HIV1455 | Light chain | VRC-PG04b | WO2013090644 SEQ ID NO: 43 | 28439 |
| HIV1456 | Light chain | Vrc-pg20 | Zhou T et al., immunity 39 (2), 245-258 (2013), | 28440 |
| | | | NCBI Accession # 4LSU_L (204aa) | |
| HIV1457 | Light chain | X5 | U.S. Pat. No. 7,378,093B2 SEQ ID NO: 2 | 28441 |
| HIV1458 | Light chain | X5 | U.S. Pat. No. 8,110,192B2 SEQ ID NO: 4 | 28442 |
| HIV1459 | Light chain | Z13e1 | Stanfield. R. L., et al, J. Mol. Biol. 414 (3), 460- | 28443 |
| | | | 476 (2011), NCBI Accession # 3Q1S_L | |
| | | | (212aa) | |
| HIV1460 | Light Chain | Z258- | Zhon T et al., Structural Repertoire of HIV-1- | 28444 |
| | | vrc27.01 | Neutralizing Antibodies Targeting the CD4 | |
| | | | Supersite in 14 Donors; Cell 161 (6), 1280- | |
| | | | 1292 (2015), NCBI Accession # 4YDI_L | |
| | | | (210aa) | |
| HIV1461 | Light chain | | NCBI Accession # 1N0X_M (215aa) | 28445 |
| HIV1462 | Light chain | | Okada, N., et al., Human IgM Monoclonal | 28446 |
| | | | Antibodies Reactive with HIV-1-Infected Cells | |
| | | | Generated Using a Trans-Chromosome Mouse; | |
| | | | Microbiol. Immunol. 49 (5), 447-459 (2005), | |
| | | | NCBI Accession # AAS01772.1(236aa) | |
| HIV1463 | Light chain | | U.S. Pat. No. 5,804,440A SEQ ID NO: 101 | 28447 |
| HIV1464 | Light chain | | U.S. Pat. No. 5,804,440A SEQ ID NO: 102 | 28448 |
| HIV1465 | Light chain | | U.S. Pat. No. 5,804,440A SEQ ID NO: 103 | 28449 |
| HIV1466 | Light chain | | U.S. Pat. No. 5,804,440A SEQ ID NO: 104 | 28450 |
| HIV1467 | Light chain | | U.S. Pat. No. 5,804,440A SEQ ID NO: 105 | 28451 |
| HIV1468 | Light chain | | U.S. Pat. No. 5,804,440A SEQ ID NO: 107 | 28452 |
| HIV1469 | Light chain | | U.S. Pat. No. 5,804,440A SEQ ID NO: 110 | 28453 |
| HIV1470 | Light chain | | U.S. Pat. No. 5,804,440A SEQ ID NO: 115 | 28454 |
| HIV1471 | Light chain | | U.S. Pat. No. 5,804,440A SEQ ID NO: 118 | 28455 |
| HIV1472 | Light chain | | U.S. Pat. No. 5,804,440A SEQ ID NO: 121 | 28456 |
| HIV1473 | Light chain | | U.S. Pat. No. 5,804,440A SEQ ID NO: 122 | 28457 |
| HIV1474 | Light chain | | U.S. Pat. No. 5,804,440A SEQ ID NO: 124 | 28458 |
| HIV1475 | Light chain | | U.S. Pat. No. 5,804,440A SEQ ID NO: 132 | 28459 |
| HIV1476 | Light chain | | U.S. Pat. No. 5,804,440A SEQ ID NO: 147 | 28460 |
| HIV1477 | Light chain | | U.S. Pat. No. 5,804,440A SEQ ID NO: 148 | 28461 |
| HIV1478 | Light chain | | U.S. Pat. No. 5,804,440A SEQ ID NO: 149 | 28462 |
| HIV1479 | Light chain | | U.S. Pat. No. 5,804,440A SEQ ID NO: 150 | 28463 |
| HIV1480 | Light chain | | U.S. Pat. No. 5,804,440A SEQ ID NO: 151 | 28464 |
| HIV1481 | Light chain | | U.S. Pat. No. 5,804,440A SEQ ID NO: 95 | 28465 |
| HIV1482 | Light chain | | U.S. Pat. No. 5,804,440A SEQ ID NO: 96 | 28466 |
| HIV1483 | Light chain | | U.S. Pat. No. 5,804,440A SEQ ID NO: 97 | 28467 |
| HIV1484 | Light chain | | U.S. Pat. No. 5,804,440A SEQ ID NO: 98 | 28468 |
| HIV1485 | Light chain | | WO2014063059 SEQ ID NO: 11 | 28469 |
| HIV1486 | Light chain | | WO2014063059 SEQ ID NO: 129 | 28470 |
| HIV1487 | Light chain | | WO2014063059 SEQ ID NO: 13 | 28471 |
| HIV1488 | Light chain | | WO2014063059 SEQ ID NO: 15 | 28472 |
| HIV1489 | Light chain | | WO2014063059 SEQ ID NO: 17 | 28473 |
| HIV1490 | Light chain | | WO2014063059 SEQ ID NO: 19 | 28474 |
| HIV1491 | Light chain | | WO2014063059 SEQ ID NO: 21 | 28475 |
| HIV1492 | Light chain | | WO2014063059 SEQ ID NO: 23 | 28476 |
| HIV1493 | Light chain | | WO2014063059 SEQ ID NO: 3 | 28477 |
| HIV1494 | Light chain | | WO2014063059 SEQ ID NO: 5 | 28478 |
| HIV1495 | Light chain | | WO2014063059 SEQ ID NO: 7 | 28479 |
| HIV1496 | Light chain | | WO2014063059 SEQ ID NO: 9 | 28480 |
| HIV1497 | Light chain | | WO2014063059 SEQ ID NO: 1 | 28481 |
| | consensus | | | |
| HIV1498 | Light chain constant | TNX-355, | US20130195881 SEQ ID NO: 2 | 28482 |
| | region | Idalizumab | | |
| HIV1499 | Light Chain Fab | Ch02 | McLellan, J. S., et al., Nature 480 (7377), 336- | 28483 |
| | | | 343 (2011), NCBI Accession # 3U46_B | |
| | | | (215aa) | |
| HIV1500 | Light Chain Of Anti- | 21C | Diskin, R., et al., Nat. Struct. Mol. Biol. 17 (5), | 28484 |
| | HIV Fab From | | 608-613 (2010), NCBI Accession # 3LMJ_L | |
| | Human 21c Antibody | | (217aa) | |
| HIV1501 | Light Chain Of Anti- | 830a | Pan et al., J. Virol. 89 (15), 8003-8010 (2015), | 28485 |
| | hiv-1 Gp120 V1v2 | | NCBI Accession # 4YWG_L (216aa) | |
| | Antibody 830a | | | |
| HIV1502 | Light Chain Of Anti- | Fab 2558 | Gorny et al., PLoS ONE 6 (12), E27780 (2011), | 28486 |
| | hiv-1 V3 Monoclonal | | NCBI Accession # 3UJI_L (209aa) | |
| | Antibody | | | |
| HIV1503 | Light Chain Of Anti- | Fab 4025 | Gorny et al., PLoS ONE 6 (12), E27780 (2011), | 28487 |
| | hiv-1 V3 Monoclonal | | NCBI Accession # 3UJJ_L (213aa) | |
| | Antibody | | | |
| HIV1504 | Light chain partial | 412D | Huang C. et al “Structural basis of tyrosine | 28488 |
| | | | sulfation and VH-gene usage in antibodies that | |
| | | | recognize the HIV type 1 coreceptor-binding | |
| | | | site on gp120” Proc. Natl. Acad. Sci. U.S.A. | |
| | | | 101 (9), 2706-2711 (2004), NCBI Accession # | |
| | | | AAR88380 | |
| HIV1505 | Light chain partial | 694/98D | Li L. et al, “A broad range of mutations in HIV- | 28489 |
| | | | 1 neutralizing human monoclonal antibodies | |
| | | | specific for V2, V3, and the CD4 binding site”, | |
| | | | Mol. Immunol. 66 (2), 364-374 (2015); NCBI | |
| | | | Accession # AKH36512 | |
| HIV1506 | Light chain variable | 0.5γ (1C10) | U.S. Pat. No. 8,722,861B2 SEQ ID NO: 2 | 28490 |
| | region | | | |
| HIV1507 | Light chain variable | 0.5γ (3D6) | U.S. Pat. No. 8,722,861B2 SEQ ID NO: 6 | 28491 |
| | region | | | |
| HIV1508 | Light chain variable | 10J4 mAb | WO2015103549 SEQ ID NO: 4 | 28492 |
| | region | | | |
| HIV1509 | Light chain variable | 10M6 mAb | WO2015103549 SEQ ID NO: 6 | 28493 |
| | region | | | |
| HIV1510 | Light chain variable | 13110 mAb | WO2015103549 SEQ ID NO: 8 | 28494 |
| | region | | | |
| HIV1511 | Light chain variable | 2N5mAb | WO2015103549 SEQ ID NO: 10 | 28495 |
| | region | | | |
| HIV1512 | Light chain variable | 35022 mAb | WO2015103549 SEQ ID NO: 2 | 28496 |
| | region | | | |
| HIV1513 | Light chain variable | 42F9 | U.S. Pat. No. 8,722,861B2 SEQ ID NO: 8 | 28497 |
| | region | | | |
| HIV1514 | Light chain variable | 49G2 | U.S. Pat. No. 8,722,861B2 SEQ ID NO: 10 | 28498 |
| | region | | | |
| HIV1515 | Light chain variable | 4O20mAb | WO2015103549 SEQ ID NO: 12 | 28499 |
| | region | | | |
| HIV1516 | Light chain variable | 5G2 | U.S. Pat. No. 8,722,861B2 SEQ ID NO: 4 | 28500 |
| | region | | | |
| HIV1517 | Light chain variable | 7B9mAb | WO2015103549 SEQ ID NO: 14 | 28501 |
| | region | | | |
| HIV1518 | Light chain variable | 7K3mAb | WO2015103549 SEQ ID NO: 16 | 28502 |
| | region | | | |
| HIV1519 | Light chain variable | B4 | U.S. Pat. No. 7,872,110B2 SEQ ID NO: 4 | 28503 |
| | region | | | |
| HIV1520 | Light chain variable | B4DIVKv.1 | U.S. Pat. No. 7,872,110B2 SEQ ID NO: 9 | 28504 |
| | region | | | |
| HIV1521 | Light chain variable | B4DIVKv.2 | U.S. Pat. No. 7,872,110B2 SEQ ID NO: 10 | 28505 |
| | region | | | |
| HIV1522 | Light chain variable | B4DIVKv.3 | U.S. Pat. No. 7,872,110B2 SEQ ID NO: 11 | 28506 |
| | region | | | |
| HIV1523 | Light chain variable | bl2 IgA2 | WO2014040024 SEQ ID NO: 30 | 28507 |
| | region | antibody | | |
| HIV1524 | Light chain variable | CHμ39.1 | U.S. Pat. No. 5,773,247 SEQ ID NO: 12 | 28508 |
| | region | | | |
| HIV1525 | Light chain variable | CHμ5.5 | U.S. Pat. No. 5,773,247 SEQ ID NO: 16 | 28509 |
| | region | | | |
| HIV1526 | Light chain variable | F425-Alg8 | WO2014040024 SEQ ID NO: 13 | 28510 |
| | region | antibody | | |
| HIV1527 | Light chain variable | Fab 43 | US20090191216 SEQ ID NO: 9 | 28511 |
| | region | | | |
| HIV1528 | Light chain variable | HGN194 | US20110212106 SEQ ID NO: 46 | 28512 |
| | region | | | |
| HIV1529 | Light chain variable | HJ16 | US20110212106 SEQ ID NO: 14 | 28513 |
| | region | | | |
| HIV1530 | Light chain variable | HK20 | US20110212106 SEQ ID NO: 30 | 28514 |
| | region | | | |
| HIV1531 | Light chain variable | IgA antibody | WO2014040024 SEQ ID NO: 15 | 28515 |
| | region | | | |
| HIV1532 | Light chain variable | Makandal | US20100111990 SEQ ID NO: 3 | 28516 |
| | region | monoclonal | | |
| | | antibody | | |
| | | (Mmab) | | |
| HIV1533 | Light chain variable | NM-01 | U.S. Pat. No. 5,665,569 SEQ ID NO: 18 | 28517 |
| | region | | | |
| HIV1534 | Light chain variable | NM-01 | U.S. Pat. No. 5,665,569 SEQ ID NO: 28 | 28518 |
| | region | HuVH | | |
| HIV1535 | Light chain variable | NM-01 | U.S. Pat. No. 5,665,569 SEQ ID NO: 30 | 28519 |
| | region | HuVK | | |
| HIV1536 | Light chain variable | NM-01 | U.S. Pat. No. 5,665,569 SEQ ID NO: 32 | 28520 |
| | region | HuVKF | | |
| HIV1537 | Light chain variable | PGT125 | Walker L. M. et al “Broad neutralization | 28521 |
| | region | | coverage of HIV by multiple highly potent | |
| | | | antibodies”, Nature 477 (7365), 466-470 | |
| | | | (2011), NCBI Accession # AEN14410 | |
| HIV1538 | Light chain variable | PGT126 | Walker L. M. et al “Broad neutralization | 28522 |
| | region | | coverage of HIV by multiple highly potent | |
| | | | antibodies”, Nature 477 (7365), 466-470 | |
| | | | (2011), NCBI Accession # AEN14411 | |
| HIV1539 | Light chain variable | PGT131 | Walker L. M. et al “Broad neutralization | 28523 |
| | region | | coverage of HIV by multiple highly potent | |
| | | | antibodies”, Nature 477 (7365), 466-470 | |
| | | | (2011), NCBI Accession # AENT4415 | |
| HIV1540 | Light chain variable | PGT136 | Walker L. M. et al “Broad neutralization | 28524 |
| | region | | coverage of HIV by multiple highly potent | |
| | | | antibodies”, Nature 477 (7365), 466-470 | |
| | | | (2011), NCBI Accession # AEN14417 | |
| HIV1541 | Light chain variable | PGT137 | Walker L. M. et al “Broad neutralization | 28525 |
| | region | | coverage of HIV by multiple highly potent | |
| | | | antibodies”, Nature 477 (7365), 466-470 | |
| | | | (2011), NCBI Accession # AEN14418 | |
| HIV1542 | Light chain variable | PGT141 | Walker L. M. et al “Broad neutralization | 28526 |
| | region | | coverage of HIV by multiple highly potent | |
| | | | antibodies”, Nature 477 (7365), 466-470 | |
| | | | (2011), NCBI Accession# AEN14419 | |
| HIV1543 | Light chain variable | PGT142 | Walker L.M. et al “Broad neutralization | 28527 |
| | region | | coverage of HIV by multiple highly potent | |
| | | | antibodies”, Nature 477 (7365), 466-470 | |
| | | | (20 1 I), NCBI Accessiots # AEN14385 | |
| HIV1544 | Light chain variable | PGT143 | Walker L.M. et al “Broad neutralization | 28528 |
| | region | | coverage of HIV by multiple liighly potent | |
| | | | antibodies”. Nature 477 (7365), 466-470 | |
| | | | (2011), NCBI Accession # AEN14421 | |
| HIV1545 | Light chain variable | PGT144 | Walker L.M. et al “Broad neutralization | 28529 |
| | region | | coverage of HIV by multiple highly potent | |
| | | | antibodies”, Nature 477 (7365), 466-470 | |
| | | | (2011), NCBI Accession # AEN14422 | |
| HIV1546 | Light chain variable | PGT151 | Falkowska, E. et al “Broadly Neutralizing HIV | 28530 |
| | region | | Antibodies Define a Gly can-Dependent Epitope | |
| | | | on the Prefusion Conformation of gp41 on | |
| | | | Cleaved Envelope Trimers” Immunity 40 (5), | |
| | | | 657-668 (2014), NCBI Accession # AIC32543 | |
| HIV1547 | Light chain variable | PGT152 | Falkowska, E. et al “Broadly Neutralizing HIV | 28531 |
| | region | | Antibodies Define a Glycan-Dependent Epitope | |
| | | | on the Prefusion Conformation of gp41 on | |
| | | | Cleaved Envelope Trimers” Immunity 40 (5), | |
| | | | 657-668 (2014), NCBI Accession # AIC32544 | |
| HIV1548 | Light chain variable | PGT153 | Falkowska, E. et al “Broadly Neutralizing HIV | 28532 |
| | region | | Antibodies Define a Glycan-Dependent Epitope | |
| | | | on the Profusion Conformation of gp41 on | |
| | | | Cleaved Envelope Trimers” Immunity 40 (>). | |
| | | | 657-668 (2014), NCBI Accession # AIC32545 | |
| HIV1549 | Light chain variable | PGT154 | Falkowska, E. et al “Broadly Neutralizing HIV | 28533 |
| | region | | Antibodies Define a Glycan-Dependent Epitope | |
| | | | on the Prefusion Conformation of gp41 on | |
| | | | Cleaved Envelope Trimers” Immunity 40 (5), | |
| | | | 657-668 (2014), NCBI Accession # AIC32529 | |
| HIV1550 | Light chain variable | PGT155 | Falkowska, E. et al “Broadly Neutralizing HIV | 28534 |
| | region | | Antibodies Define a Glycan-Dependent Epitope | |
| | | | on the Prefusion Conformation of gp41 on | |
| | | | Cleaved Envelope Trimers” Immunity 40 (5), | |
| | | | 657-668 (2014), NCBI Accession # AIC32547 | |
| HIV1551 | Light chain variable | PGT156 | Falkowska, E. et al “Broadly Neutralizing HIV | 28535 |
| | region | | Antibodies Define a Glycan-Dependent Epitope | |
| | | | on the Prefusion Conformation of gp41 on | |
| | | | Cleaved Envelope Trimers” Immunity 40 (5), | |
| | | | 657-668 (2014), NCBI Accession # AIC32548 | |
| HIV1552 | Light chain variable | PGT157 | Falkowska, E. et al “Broadly Neutralizing HIV | 28536 |
| | region | | Antibodies Define a Glycan-Dependent Epitope | |
| | | | on the Prefusion Conformation of gp41 on | |
| | | | Cleaved Envelope Trimers” Immunity 40 (5), | |
| | | | 657-668 (2014), NCBI Accession # AIC32549 | |
| HIV1553 | Light chain variable | PGTI58 | Falkowska, E. et al “Broadly Neutralizing HIV | 28537 |
| | region | | Antibodies Define a Glycan-Dependent Epitope | |
| | | | on the Prefusion Conformation of gp41 on | |
| | | | Cleaved Envelope Trimers” Immunity 40 (5), | |
| | | | 657-668 (2014), NCBI Accession # AIC32550 | |
| HIV1554 | Light chain variable | rF105 | WO1993012232 SEQ ID NO: 3 | 28538 |
| | region | | | |
| HIV1555 | Light chain variable | ScFvX5-CD4 | U.S. Pat. No. 7,378,093B2 SEQ ID NO: 15 | 28539 |
| | region | | | |
| HIV1556 | Light chain variable | TNX-355, | US20130195881 SEQ ID NO: 1 | 28540 |
| | region | Idalizumab | | |
| HIV1557 | Light chain variable | VCR14 | US20150044137 SEQ ID NO: 25 | 28541 |
| | region | | | |
| HIV1558 | Light chain variable | VCR14b | US20150044137 SEQ ID NO: 26 | 28542 |
| | region | | | |
| HIV1559 | Light chain variable | VCR14c | US20150044137 SEQ ID NO: 27 | 28543 |
| | region | | | |
| HIV1560 | Light chain variable | VCR16 | US20150044137 SEQ ID NO: 33 | 28544 |
| | region | | | |
| HIV1561 | Light chain variable | VCR16b | US20150044137 SEQ ID NO: 34 | 28545 |
| | region | | | |
| HIV1562 | Light chain variable | VCR16c | US20150044137 SEQ ID NO: 35 | 28546 |
| | region | | | |
| HIV1563 | Light chain variable | VCR16d | US20150044137 SEQ ID NO: 36 | 28547 |
| | region | | | |
| HIV1564 | Light chain variable | VRC13 | US20150044137 SEQ ID NO: 17 | 28548 |
| | region | | | |
| HIV1565 | Light chain variable | VRC13b | US20150044137 SEQ ID NO: 18 | 28549 |
| | region | | | |
| HIV1566 | Light chain variable | VRC13c | US20150044137 SEQ ID NO: 19 | 28550 |
| | region | | | |
| HIV1567 | Light chain variable | VRC13d | US20150044137 SEQ ID NO: 20 | 28551 |
| | region | | | |
| HIV1568 | Light chain variable | VRC13e | US20150044137 SEQ ID NO: 21 | 28552 |
| | region | | | |
| HIV1569 | Light chain variable | VRC13f | US20150044137 SEQ ID NO: 22 | 28553 |
| | region | | | |
| HIV1570 | Light chain variable | VRC13g | US20150044137 SEQ ID NO: 23 | 28554 |
| | region | | | |
| HIV1571 | Light chain variable | VRC13h | US20150044137 SEQ ID NO: 24 | 28555 |
| | region | | | |
| HIV1572 | Light chain variable | VRC15 | US20150044137 SEQ ID NO: 28 | 28556 |
| | region | | | |
| HIV1573 | Light chain variable | | US20150004190 SEQ ID NO: 57 | 28557 |
| | region | | | |
| HIV1574 | Light Chain, Fab | Ch04 | McLellan, J. S. et al., Structure of HIV-1 gp120 | 28558 |
| | | | V1 V2 domain with broadly neutralizing | |
| | | | antibody PC9; Nature 480 (7377), 336-343 | |
| | | | (2011), NCBI Accession # 3TCL_B (215aa) | |
| HIV1575 | Light Chain, Fab | N5-i5 | Acharya, P., et al., Structural Definition of an | 28559 |
| | | | Antibody-Dependent Cellular Cytotoxicity | |
| | | | Response Implicated in Reduced Risk for HIV- | |
| | | | 1 Infection; J. Virol. 88 (21), 12895-12906 | |
| | | | (2014), NCBI Accession # 4H8W_L (217aa) | |
| HIV1576 | Light Chain, Ig | Nih45-46 Fab | Diskin, R., et al., Science 334 (6060), 1289- | 28560 |
| | Kappa Chain C | | 1293 (2011), NCBI Accession # 3U7Y_L | |
| | Region | | (210aa) | |
| HIV1577 | Light Chain, Ig | Pgt127 | Pejchal, R., et al., Science 334 (6059), 1097- | 28561 |
| | Lambda-2 Chain C | | 1103 (2011), NCBI Accession # | |
| | region | | 3TWC_L(211aa) | |
| HIV1578 | Light Chain, Ig | 7b2 | Santra, S., et al., PLoS Pathol. 11 (8), | 28562 |
| | Kappa Chain C | | E1005042 (2015), NCBI Accession # 4YDV_L | |
| | Region | | (265aa) | |
| HIV1579 | Light Chain; Fab | N60-i3 | Gohain, N., et al., Cocrystal Structures of | 28563 |
| | | | Antibody N60-i3 and Antibody JR4 in | |
| | | | Complex with gp120 Define More Cluster A | |
| | | | Epitopes Invoked in Effective Antibody- | |
| | | | Dependent Effector Function against HIV-1; J. | |
| | | | Virol. 89 (17), 8840-8854 (2015), NCBI | |
| | | | Accession # 4RFO_L (221aa) | |
| HIV1580 | Light Chain; Ig | Pgt128 | Pejchal, R., et al., Science 334 (6059), 1097- | 28564 |
| | Lambda-2 Chain C | | 1103 (2011), NCBI Accession # 3TV3_L | |
| | region | | (211aa) | |
| HIV1581 | Scfv | B11 | U.S. Pat. No. 7,744,887B2 SEQ ID NO: 8 | 28565 |
| HIV1582 | Scfv | | U.S. Pat. No. 8,110,192B2 SEQ ID NO: 1 | 28566 |
| HIV1583 | Scfv | | U.S. Pat. No. 8,110,192B2 SEQ ID NO: 2 | 28567 |
| HIV1584 | Scfv | | U.S. Pat. No. 8,110,192B2 SEQ ID NO: 3 | 28568 |
| HIV1585 | Scfv (SEQRES) | 3b3 variant | Clark et al., Protein Sci. 18 (12), 2429-2441 | 28569 |
| | | | (2009), NCBI Accession # 3JUY_A (256aa) | |
| HIV1586 | Scfv | D5 | U.S. Pat. No. 7,744,887B2 SEQ ID NO: 2 | 28570 |
| HIV1587 | Scfv-cd4 fusion | | U.S. Pat. No. 8,110,192B2 SEQ ID NO: 8 | 28571 |
| | protein | | | |
| HIV1588 | | 447-52d | Dhillon, A. K., et al., Acta Crystallogr. D Biol. | 28572 |
| | | | Crystallogr. D64 (PT 7), 792-802 (2008), NCBI | |
| | | | Accession # 3C2A_1(231aa) | |
| HIV1589 | | 447-52d | Dhillon, A. K., et al., Acta Crystallogr, D Biol. | 28573 |
| | | | Crystallogr. D64 (PT 7), 792-802 (2008), NCBI | |
| | | | Accession # 3C2A_M (216aa) | |
| HIV1590 | | F105 | Wilkinson, R. A., et al., J. Virol. 79 (20), 13060- | 28574 |
| | | | 13069 (2005), NCBI Accession # 1U6A_H | |
| | | | (224aa) | |
| HIV1591 | | F105 | Wilkinson, R. A., et al., J. Virol. 79 (20), 13060- | 28575 |
| | | | 13069 (2005), NCBI Accession # 1U6A_L | |
| | | | (215aa) | |
| HIV1592 | | Fab 8062 | Frisch, C., et al., PLoS Pathol. 6 (11), | 28576 |
| | | | E1001182 (2010), NCBI Accession # 3MAC_H | |
| | | | (245aa) | |
| HIV1593 | | Fab 8062 | Frisch, C., et al., PLoS Pathol. 6 (11), | 28577 |
| | | | E1001182 (2010), NCBI Accession # 3MAC_L | |
| | | | (213aa) | |
| HIV1594 | | Fab 8066 | Frisch, C., et al., PLoS Pathol. 6 (11), | 28578 |
| | | | E1001182 (2010), NCBI Accession # 3MA9_H | |
| | | | (245aa) | |
| HIV1595 | | Fab 8066 | Frisch, C., et al., PLoS Pathol. 6 (11), | 28579 |
| | | | E1001182 (2010), NCBI Accession # 3MA9_L | |
| | | | (213aa) | |
| HIV1596 | | Fab′2F5 | U.S. Pat. No. 6,482,928 SEQ ID NO: 6 | 28580 |
| | | fragment | | |
| HIV1597 | | Fab′2F5 | U.S. Pat. No. 6,482,928 SEQ ID NO: 7 | 28581 |
| | | fragment | | |
| HIV1598 | | M18 Fab | Prabakaran, P., et al., J. Mol. Biol. 357 (1), 82- | 28582 |
| | | | 99 (2006), NCBI Accession # 2AJ3_D (228aa) | |
| HIV1599 | | M18 Fab | Prabakaran, P., et al., J. Mol. Biol. 357 (1), 82- | 28583 |
| | | | 99 (2006), NCBI Accession # 2AJ3_E (213aa) | |
| HIV1600 | | Pg16 | Pancera, M. et al., J. Virol. 84 (16), 8098-8110 | 28584 |
| | | | (2010), NCBI Accession # 3MME_A (238aa) | |
| HIV1601 | | Pg16 | Paneera, M, et al., J. Virol. 84 (16), 8098-8110 | 28585 |
| | | | (2010), NCBI Accession # 3MME_B (216aa) | |

In some embodiments, the payload region of the viral particle comprises one or more nucleic acid sequences, fragments or variants thereof or encodes one or more polypeptides, fragments or variants thereof described in European Patent Publication No. EP327000, EP478689, EP554401, EP581353 and EP711439, US Publication No. US20110104163, US20110212106, 0520130215726 and US20130251726, U.S. Pat. Nos. 5,266,479, 5,804,440, 6,657,050, 8,637,036, and 9,090,675, and International Publication No. WO2012154312, WO2013163427, WO2014043386, WO2015048462, WO2015048610, WO2015048770 the contents of each of which are herein incorporated by reference in their entirety, against HIV.

AAV Particles Comprising TRIM21 payloads

In some embodiments, the payload region of the viral particle comprises one or more nucleic acid sequences encoding TRIM21, variants or fragments thereof.

In some embodiments, the viral particles are useful in the field of medicine for the treatment, prophylaxis, palliation or amelioration of neurological diseases and/or disorders.

In some embodiments, the viral particles are AAV particles, comprising a viral genome and a capsid.

Non-limiting examples of ITR to ITR sequences of AAV particles comprising a viral genome with a payload region comprising a TRIM21 polynucleotide sequence are described in Table 54,

TABLE 54
Representative ITR to ITR Sequences of AAV
particles comprising TRIM21 sequences

| ITR to ITR | Sequence | ITR to ITR |
|---|---|---|
| Construct Name | Length (nt) | SEQ ID NO |
| TRIM21_ITR1 | 3590 | 32672 |
| TRIM21_ITR2 | 3629 | 32673 |
| TRIM21_ITR3 | 3638 | 32674 |
| TRIM21_ITR4 | 4307 | 32675 |

In some embodiments, the AAV particle comprises a viral genome which comprises a sequence that has a percent identity to any of SEQ ID NO: 32672-32675, The viral genome may have 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99% or 100% identity to any of SEQ NOs: 32672-32675. The viral genome may have 1-10%, 10-20%, 30-40%, 50-60%, 50-70%, 50-80%, 50-90%, 50-99%, 50-100%, 60-70%, 60-80%, 60-90%, 60-99%, 60100%, 70-80%, 70-90%, 70-99%, 70-100%, 80-85%, 80-90%, 80-95%, 80-99%, 80-100%, 90-95%, 90-99%, or 90-100% to any of SEQ ID NOs: 32672-32675. As a non-limiting example, the viral genome comprises a sequence which has 80% identity to any of SEQ ID NO: 32672-32675. As another non-limiting example, the viral genome comprises a sequence which as 85% identity to any of SEQ ID NO: 32672-32675, As another non-limiting example, the viral genome comprises a sequence which as 90% identity to any of SEQ ID NO: 32672-32675. As another non-limiting example, the viral genome comprises a sequence which as 95% identity to any of SEQ ID NO: 32672-32675. As another non-limiting example, the viral genome comprises a sequence which as 99% identity to any of SEQ ID NO: 32672-32675.

In some embodiments, the AAV particles comprising a TRIM21 polynucleotide comprise one or more components such as, but not limited to, a 5′ ITR, a promoter, an exon region, an intron region, a tag, a TRIM21 polynucleotide, a poly(A) sequence and a 3′ ITR.

In certain embodiments the viral genome may also encode one or more antibody polynucleotides as described herein.

In some embodiments, the AAV particle viral genome may comprise one or more sequences as described in Table 55 below. The regions may be located before or after any of the other sequence regions described herein. Viral genomes may further comprise more than one copy of one or more sequence regions as described in Table 55.

TABLE 55
Representative viral genome component sequences

| Component | Sequence | Component |
|---|---|---|
| Name | Length (nt) | SEQ ID NO |
| ITR1 | 141 | 32676 |
| PROMOTER1 | 654 | 32677 |
| EXON1 | 134 | 32678 |
| INTRON1 | 32 | 32679 |
| INTRON2 | 347 | 32680 |
| EXON2 | 53 | 32681 |
| TAG1 | 27 | 32682 |
| TAG2 | 18 | 32683 |
| TAG3 | 21 | 32684 |
| TAG4 | 708 | 32685 |
| TRIM21_1 | 1428 | 32686 |
| POLYA1 | 477 | 32687 |
| ITR2 | 141 | 32688 |

In some embodiments, the AAV particle viral genome may comprise at least one inverted terminal region (ITR) region. The ITR region(s) may, independently, have a length such as, but not limited to, 75, 6, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, and 175 nucleotides. The length of the ITR. region for the viral genome may be 75-80, 75-85, 75-100, 80-85, 80-90, 80-105, 85-90, 85-95, 85-110, 90-95, 90-100, 90115, 95-100, 95-105, 95-120, 100-105, 100-110, 100-125, 105-110, 1105-115, 1105-130, 1110-115, 110-120, 110-135, 115-120, 115-125, 115-140, 120-125, 120-130, 120-145, 125-130, 1125-135, 125-150, 130-135, 130-140, 130-155, 135-140, 135-145, 135-160, 140-145, 140-150, 140-165, 145-150, 145-155, 145-170, 150-155, 150-160, 150-175, 155-160, 155-165, 160-1165, 160-1170, 165-170, 165-175, and 170-175 nucleotides. As a non-limiting example, the viral genome comprises a 5′ ITR that is about 141 nucleotides in length. As a non-limiting example, the viral genome comprises a 5′ ITR that is about 130 nucleotides in length. As a non-limiting example, the viral genome comprises a 5′ ITR that is about 119 nucleotides in length. As a non-limiting example, the viral genome comprises a 3′ ITR that is about 141 nucleotides in length. As a non-limiting example, the viral genome comprises a 3′ ITR that is about 130 nucleotides in length. As a non-limiting example, the viral genome comprises a 3′ ITR that is about 1119 nucleotides in length. As a non-limiting example, the 5′ ITR and the 3′ ITR may comprise the same length and/or the same sequence. In another non-limiting example, the 5′ 11′R and the 3′1717R are different in length and/or in sequence.

In some embodiments, the at least one ITR may be ITR1 (SEQ ID NO: 32676). In some embodiments, the at least one ITR may be ITR2 (SEQ ID NO: 32688). In some embodiments, the AAV particle viral genome comprises two ITR regions. As a non-limiting example, the ITR regions may be ITR1 and ITR2. In some embodiments, the 5′ ITR, comprises ITR1 and the 3′ ITR comprises ITR2.

In some embodiments, the AAV particle viral genome may comprise at least one promoter sequence region. The promoter sequence region(s) may, independently, have a length such as, but not limited to, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600 and more than 600 nucleotides. The length of the promoter region for the viral genome may be 4-10, 10-20, 10-50, 20-30, 30-40, 40-50, 50-60, 50-100, 60-70, 70-80, 80-90, 90-100, 100-110, 100-150, 110-120, 120-130, 130-140, 140-150, 150-160, 150-200, 160-470, 170-480, 180-190, 190-200, 200-210, 200-250, 210-220, 220-230, 230-240, 240-250, 250-260, 250-300, 260-270, 270-280, 280-290, 290-300, 300-310, 300-350, 310-320, 320-330, 330-340, 340-350, 350-360, 350-400, 360-370, 370-380, 380-390, 390-400, 400-410, 400-450, 410-420, 420-430, 430-440, 440-450, 450-460, 450-500, 460-470, 470-480, 480-490, 490-500, 500-510, 500-550, 510520, 520530, 530540, 540-550, 550-560, 550-600, 560-570, 570-580, 580-590, 590-600 and more than 600 nucleotides. As a non-limiting example, the viral genome comprises a promoter region that is about 654 nucleotides in length.

In certain embodiments, the AAV particle viral genome comprises one promoter sequence region. In certain embodiments, the promoter sequence region is PROMOTER1 (SEQ ID NO: 32677).

In some embodiments, the AAV particle viral genome promoter sequence region comprises a chicken beta-actin (CBA) promoter or variant or derivative thereof.

In some embodiments, the AAV particle viral genome may comprise more than one promoter sequence region. In some embodiments, the AAV particle viral genome comprises two promoter sequence regions. In some embodiments, the AAV particle viral genome comprises more than two promoter sequence regions.

In some embodiments, the AAV particle viral genome may comprise at least one intron and/or exon sequence region.

In some embodiments, the AAV particle viral genome may comprise at least one intron sequence region. The intron sequence region(s) may, independently, have a length such as, but not limited to, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 11.1, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, and more than 350 nucleotides. The length of the intron sequence region for the viral genome may be 25-35, 25-50, 35-45, 45-55, 50-75, 5565, 65-75, 75-85, 75100, 85-95, 95-105, 100-125, 105-115, 115-125, 125-135, 125-150, 135-145, 145-155, 150-175, 155-165, 165-175, 175-185, 175-200, 185-195, 195-205, 200-225, 205-215, 215-225, 225-235, 225-250, 235-245, 245-255, 250-275, 255-265, 265-275, 275-285, 275-300, 285-295, 295-305, 300-325, 305-315, 315325, 325335, 325-350, and 335-345 nucleotides. As a non-limiting example, the viral genome comprises an intron sequence region that is about 32 nucleotides in length. As a non-limiting example, the viral genome comprises an intron sequence region that is about 53 nucleotides in length. As a non-limiting example, the viral genome comprises an intron sequence region that is about 134 nucleotides in length. As a non-limiting example, the viral genome comprises an intron sequence region that is about 347 nucleotides in length. As a non-limiting example, the viral genome comprises an intron sequence region that is about 379 nucleotides in length. As a non-limiting example, the viral genome comprises an intron sequence region that is about 566 nucleotides in length.

In some embodiments, the AAV particle viral genome comprises two intron sequence regions. In some embodiments, the AAV particle viral genome comprises three intron sequence regions. In some embodiments, the AAV particle viral genome comprises more than three intron sequence regions.

In some embodiments, the AAV particle viral genome comprises INTRON1 (SEQ ID NO: 32679). In some embodiments, the AAV particle viral genome comprises INTRON2 (SEQ ID NO: 32680), In some embodiments, the AAV particle viral genome comprises INTRON 1 and INTRON2. In some embodiments, one or more intron sequences are derived from an ie1 (CMV immediate early) gene. In some embodiments, one or more intron sequences are derived from a human beta-globin gene.

In some embodiments, the AAV particle viral genome may comprise at least one exon sequence region. The exon sequence region(s) may, independently, have a length such as, but not limited to, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, and more than 350 nucleotides. The length of the exon sequence region for the viral genome may be 25-35, 25-50, 3545, 4555, 50-75, 55-65, 65-75, 75-85, 75-100, 8595, 95-105, 100-125, 105-115, 115-125, 125-135, 125-150, 135-145, 145-155, 150-175, 155-165, 165-175, 175-185, 175-200, 185-195, 195-205, 200-225, 205-215, 215-225, 225-235, 225-250, 235-245, 245-255, 250275, 255265, 265-275, 275-285, 275-300, 285-295, 295-305, 300-325, 305-315, 315-325, 325-335, 325-350, and 335-345 nucleotides. As a non-limiting example, the viral genome comprises an exon region that is about 32 nucleotides in length. As a non-limiting example, the viral genome comprises an exon sequence region that is about 53 nucleotides in length. As a non-limiting example, the viral genome comprises an exon sequence region that is about 134 nucleotides in length. As a non-limiting example, the viral genome comprises an exon sequence region that is about 347 nucleotides in length. As a non-limiting example, the viral genome comprises an exon sequence region that is about 379 nucleotides in length. As a non-limiting example, the viral genome comprises an exon sequence region that is about 566 nucleotides in length.

In some embodiments, the AAV particle viral genome comprises two exon sequence regions. In some embodiments, the AAV particle viral genome comprises three exon sequence regions. In some embodiments, the AAV particle viral genome comprises more than three exon sequence regions.

In some embodiments, the AAV particle viral genome comprises EXON1 (SEQ ID NO. 32678). In some embodiments, the AAV particle viral genome comprises EXON2 (SEQ ID NO: 32681). In some embodiments, the AAV particle viral genome comprises EXON: 1 and EXON2. In some embodiments, one or more exon sequences are derived from an ie1 (CMV immediate early) gene. In some embodiments, one or more exon sequences are derived from a human beta-globin gene.

In some embodiments, the AAV particle viral genome comprises a hybrid intron/exon sequence region comprising at least one intron and at least one exon. In some embodiments, the hybrid intron/exon sequence region comprises one intron and one exon. In some embodiments, the hybrid intron/exon sequence region comprises two introns and two exons. In some embodiments, an intron or exon sequence may comprise a full length intron or exon. In some embodiments, an intron or exon sequence may comprise a fragment or variant of an intron or exon sequence.

The hybrid intron/exon sequence region(s) may, independently, have a length such as, but not limited to, 15-100, 100-200, 200-300, 300-400, 400-500, 500-600, 600-700, 700-800, 800-900, 900-1000, 1000-1100, 1100-1200, and more than 1200 nucleotides. As a non-limiting example, the viral genome comprises a hybrid intron/exon sequence region that is about 379 nucleotides in length. As a non-limiting example, the viral genome comprises a hybrid intron/exon sequence region that is about 566 nucleotides in length. As a non-limiting example, the viral genome comprises a hybrid intron/exon region that is about 379 nucleotides in length.

In some embodiments, the hybrid intron/exon sequence region may comprise one or more of EXON1 (SEQ ID NO: 32678), EXON2 (SEQ ID NO: 32681), INTRON1 (SEQ ID NO: 32679) and/or INTRON2 (SEQ ID NO: 32680), In some embodiments, the hybrid intron/exon sequence region, when read 5′ to 3′ comprises EXON1, INTRON1, INTRON2, and EXON2.

In some embodiments, the intron/exon sequence region is an enhancer sequence. In some embodiments, the intron/exon sequence region is not an enhancer sequence.

As used herein, an “enhancer” or “enhancer sequence” refers to an element or component of the viral genome used to enhance the target specificity and/or expression of the payload (e.g., antibody or TRIM21). In some embodiments, a promoter may comprise an enhancer sequence. In some embodiments, an enhancer may be a separate component of the viral genome than the promoter. In some embodiments, an enhancer may be 5′ to a promoter sequence in a viral genome. In some embodiments, an enhancer may be 3′ to a promoter sequence in a viral genome.

In some embodiments, the intron/exon sequence region is a component of a promoter sequence. In some embodiments, the intron/exon sequence region is not a component of a promoter sequence.

In some embodiments, the AAV particle viral genome comprises at least one tag sequence region. As used herein, the term “tag” indicates a polynucleotide sequence appended to the payload (5′ or 3′), that once expressed may be used to identify the expressed payload. Alternatively, the term “tag” may indicate a polynucleotide sequence appended to the payload (5′ or 3′) that signals for retention of the expressed payload in a particular region of the cell (e.g., endoplasmic reticulum).

The tag sequence region(s) may, independently, have a length such as, but not limited to, 10, 11, 12, 13, 14, 15, 16, 17, 18, 11.9, 20, 21, 22, 23, 24, 25, 26, 27, 28, 29, 30 or more than 30 nucleotides. The length of the tag sequence region in the viral genome may be 10-15, 15-20, 20-25, 25-30, or more than 30 nucleotides. As a non-limiting example, the viral genome comprises a tag sequence region that is about 18 nucleotides in length. As a non-limiting example, the viral genome comprises a tag sequence region that is about 21 nucleotides in length. As a non-limiting example, the viral genome comprises a tag sequence region that is about 27 nucleotides in length. As a non-limiting example, the viral genome comprises a tag sequence region that is about 39 nucleotides in length. As a non-limiting example, the viral genome comprises a tag sequence region that is about 708 nucleotides in length.

In some embodiments, the AAV particle viral genome comprises one tag sequence region. In some embodiments, the AAV particle viral genome comprises a tag sequence region given as TAG1 (SEQ ID NO: 32682). In some embodiments, the AAV particle viral genome comprises a tag sequence region given as TAG2 (SEQ ID NO: 32683). In some embodiments, the AAV particle viral genome comprises a tag sequence region given as TAG3 (SEQ ID NO: 32684). In some embodiments, the AAV particle viral genome comprises a tag sequence region given as TAG4 (SEQ ID NO: 32685). In some embodiments, the tag sequence region is a Human influenza hemagglutinin (HA) tag. In some embodiments, the tag sequence region a His tag. In some embodiments, the tag sequence region comprises more than one His sequence repeated. In some embodiments, the tag sequence region comprises six repeats of a His tag sequence. In some embodiments, the tag sequence region comprises a TEV (Tobacco Etch Virus protease) site. In some embodiments, the tag sequence region comprises an mcherry sequence.

In some embodiments, the AAV particle viral genome comprises more than one tag sequence region. In some embodiments, the AAV particle viral genome comprises two tag sequence regions. In some embodiments, the AAV particle viral genome comprises tag sequence regions TACI2 and TAG3. In some embodiments, the AAV particle viral genome comprises three tag sequence regions. In some embodiments, the AAV particle viral genome comprises more than three tag sequence regions.

In some embodiments, the AAV particle viral genome comprises one or more TRIM21 sequence regions. In some embodiments, the AAV particle viral genome comprises a TRIM21 sequence region given as TRIM21_1 (SEQ ID NO: 32686). In some embodiments, the AAV particle viral genome comprises a TRIM21 sequence region given as SEQ ID NO: 32670 or a fragment, variant or derivative thereof. In some embodiments, the TRIM21 sequence region may be 1428 nucleotides in length.

In some embodiments, the AAV particle viral genome may comprise at least one polyadenylation (polyA) sequence region. The polyadenylation sequence region(s) may, independently, have a length such as, but not limited to, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, and 600 nucleotides. The length of the polyadenylation sequence region for the viral genome may be 4-10, 10-20, 10-50, 20-30, 3040, 40-50, 50-60, 50-100, 60-70, 70-80, 80-90, 90-100, 100-110, 100-150, 110-120, 120-130, 130-140, 140-150, 150-160, 150-200, 160-170, 170-180, 180-190, 190-200, 200210, 200250, 210 220, 220-230, 230-240, 240-250, 250-260, 250-300, 260-270, 270-280, 280-290, 290-300, 300-310-300-350, 310-320, 320-330, 330-340, 340-350, 350-360, 350-400, 360-370, 370-380, 380-390, 390-400, 400-410, 400-450, 410-420, 420-430, 430-440, 440-450, 450-460, 450-500, 460-470, 470-480, 480-490, 490-500, 500-510, 500-550, 510-520, 520-530, 530-540, 540-550, 550 560, 550-600, 560-570, 570-580, 580-590, and 590-600 nucleotides. In some embodiments, the viral genome comprises a polyadenylation sequence region that is about 477 nucleotides in length.

In some embodiments, the AAV particle viral genome comprises at least one polyA sequence region. In some embodiments, the AAV particle viral genome comprises one polyA sequence region. In some embodiments, the AAV particle viral genome comprises a polyA sequence region given as POLYA1 (SEC.) ID NO: 32687). As a non-limiting example, the polyA sequence comprises a human growth hormone polyadenylation sequence.

In some embodiments, the AAV particle viral genome comprises more than one polyA sequence region.

In some embodiments, the AAV particle viral genome may comprise any of the sequences shown in Table 56.

TABLE 56
Representative sequence regions in ITR to ITR sequences

| | TRIM21_ITR1 | | TRIM21_ITR2 | | TRIM21_ITR3 | | TRIM21_ITR4 | |
|---|---|---|---|---|---|---|---|---|
| | (SEQ ID NO: | | (SEQ ID NO: | | (SEQ ID NO: | | (SEQ ID NO: | |
| | 32672) | | 32673) | | 32674) | | 32675) | |
| | Region | | Region | | Region | | Region | |
| | SEQ | Region | SEQ | Region | SEQ | Region | SEQ | Region |
| Sequence Regions | ID NO | length | ID NO | length | ID NO | length | ID NO | length |
| 5′ ITR | 32676 | 141 | 32676 | 141 | 32676 | 141 | 32676 | 141 |
| Promoter | 32677 | 654 | 32677 | 654 | 32677 | 654 | 32677 | 654 |
| Exon | 32678 | 134 | 32678 | 134 | 32678 | 134 | 32678 | 134 |
| Intron | 32679 | 32 | 32679 | 32 | 32679 | 32 | 32679 | 32 |
| Intron | 32680 | 347 | 32680 | 347 | 32680 | 347 | 32680 | 347 |
| Exon | 32681 | 53 | 32681 | 53 | 32681 | 53 | 32681 | 53 |
| Tag | — | — | 32682 | 27 | 32683 | 18 | 32685 | 708 |
| Tag | — | — | — | — | 32684 | 21 | — | — |
| TRIM21 | 32686 | 1428 | 32686 | 1428 | 32686 | 1428 | 32686 | 1428 |
| PolyA | 32687 | 477 | 32687 | 477 | 32687 | 477 | 32687 | 477 |
| 3′ ITR | 32688 | 141 | 32688 | 141 | 32688 | 141 | 32688 | 141 |

In some embodiments, the AAV particle viral genome comprises SEQ ID NO: 32672 (TRIM21_ITR1), which comprises a 5′ inverted terminal repeat (ITR) sequence region and a 3′ ITR sequence region, a CBA promoter region, a human beta globin intron/exon region comprising an ie1 exon 1 region, an ie1 intron 1 region, a human beta-globin intron region, a human-beta-globin exon region, a TRIM21 sequence region, and a human growth hormone polyadenylation sequence.

In some embodiments, the AAV particle viral genome comprises SEQ ID NO: 32673 (TRIM21_ITR2), which comprises a 5′ inverted terminal repeat (ITR) sequence region and a 3′ ITR sequence region, a CBA promoter region, a human beta globin intron/exon region comprising an ie1 exon 1 region, an ie1 intron 1 region, a human beta-globin intron region, a human beta-globin exon region, an HA tag sequence region, a TRIM sequence region, and a human growth hormone polyadenylation sequence.

In some embodiments, the AAV particle viral genome comprises SEQ ID NO: 32674 (TRINE′ ITR3), which comprises a 5′ inverted terminal repeat (ITR) sequence region and a 3′ ITR sequence region, a CBA promoter region, a human beta globin intron/exon region comprising an ie1 exon 1 region, an ie1 intron 1 region, a human beta-globin intron region, a human beta globin exon region, a His tag sequence region, a TEV site, a TRIM21 sequence region, and a human growth hormone polyadenylation sequence.

In some embodiments, the AAV particle viral genome comprises SEQ ID NO: 32675 (TRIM21_ITR4), which comprises a 5′ inverted terminal repeat (ITR) sequence region and a 3′ ITR sequence region, a CBA promoter region, a human beta globin intron/exon region comprising an ie1 exon 1 region, an ie1 intron 1 region, a human beta-globin intron region, a human-beta-globin exon region, an mCherry tag sequence region, a TRIM21 sequence region, and a human growth hormone polyadenylation sequence.

Certain embodiments provide the viral genome as packaged in a capsid having a serotype selected from Table 1, or any capsid known in the art. For example, the capsid serotype may be selected from the group consisting of VOY101, VOY201, AAVPHP.B, AAVPHP.N, AAV1, AAV2, AAV3, AAV4, AAV5, AAV6, AAV7, AAV8, AAV9, AAV9.47, AAV9(hu14), AAV9 K449R, AAV10, AAV11, AAV12, AAVrh8, AAVrh10, ARVIN, and AAVDJ8, or any variant thereof.

In some embodiments a viral genome as provided in Tables 54-56 is packaged into an AAV capsid to generate an AAV particle. In some embodiments, the AAV particle comprises a viral genome comprising SEQ ID NO: 32672 and a VOY101 capsid. In some embodiments, the AAV particle comprises a viral genome comprising SEQ ID NO: 32672 and a VOY201 capsid. In some embodiments, the AAV particle comprises a viral genome comprising SEQ ID NO: 32672 and an AAV9 capsid. In some embodiments, the AAV particle comprises a viral genome comprising SEQ ID NO: 32672 and an AAV9 K449R. capsid. In some embodiments, the AAV particle comprises a viral genome comprising SEQ ID NO: 32672 and an AAVPHP.B capsid. In some embodiments, the AAV particle comprises a viral genome comprising SEQ ID NO: 32672 and an AAVPHP.N capsid.

In some embodiments a viral genome as provided in Tables 54-56 is packaged into an AAV capsid to generate an AAV particle. In some embodiments, the AAV particle comprises a viral genome comprising SEQ ID NO: 32673 and a VOY101 capsid. In some embodiments, the AAV particle comprises a viral genome comprising SEQ ID NO: 32673 and a VOY201 capsid. In some embodiments, the AAV particle comprises a viral genome comprising SEQ ID NO: 32673 and an AAV9 capsid. In some embodiments, the AAV particle comprises a viral genome comprising SEQ ID NO: 32673 and an AAV9 K449R capsid. In some embodiments, the AAV particle comprises a viral genome comprising SEQ ID NO: 32673 and an AAVPHP.B capsid. In some embodiments, the AAV particle comprises a viral genome comprising SEQ ID NO: 32673 and an AAVPHP.N capsid.

In some embodiments a viral genome as provided in Tables 54-56 is packaged into an AAV capsid to generate an AAV particle. In some embodiments, the AAV particle comprises a viral genome comprising SEQ ID NO: 32674 and a VOY101 capsid. In some embodiments, the AAV particle comprises a viral genome comprising SEQ ID NO: 32674 and a VOY201 capsid. In some embodiments, the AAV particle comprises a viral genome comprising SEQ ID NO: 32674 and an AAV9 capsid. In some embodiments, the AAV particle comprises a viral genome comprising SEQ ID NO: 32674 and an AAV9 K449R capsid. In some embodiments, the AAV particle comprises a viral genome comprising SEQ ID NO: 32674 and an AAVPHP.B capsid. In some embodiments, the AAV particle comprises a viral genome comprising SEQ ID NO: 32674 and an AAVPHRN capsid.

In some embodiments a viral genome as provided in Tables 54-56 is packaged into an AAV capsid to generate an AAV particle. In some embodiments, the AAV particle comprises a viral genome comprising SEQ ID NO: 32675 and a VOY101 capsid. In some embodiments, the AAV particle comprises a viral genome comprising SEQ ID NO: 32675 and a VOY201. capsid. In some embodiments, the AAV particle comprises a viral genome comprising SEQ ID NO: 32675 and an AAV9 capsid. In some embodiments, the AAV particle comprises a viral genome comprising SEQ ID NO: 32675 and an AAV9 K449R capsid. In some embodiments, the AAV particle comprises a viral genome comprising SEQ ID NO: 32675 and an AAVPHP.B capsid. In some embodiments, the AAV particle comprises a viral genome comprising SEQ ID NO: 32675 and an AAVPHP.N capsid.

### Chimeric Antigen Receptor

In some embodiments, payloads of the present disclosure may be a chimeric antigen receptor (CAR), which when transduced into immune cells (e.g., T cells and NK cells), can re-direct the immune cells against the target (e.g., a tumor cell) which expresses a molecule recognized by the extracellular target moiety of the CAR. The expression of such payloads may be augmented using the VA-DER systems of the disclosure whereby either TRIM21 as a protein or vectored TRIM21 is also delivered or administered with the CAR or with an antibody which recognizes the CAR. Cells engineered to express CARs may also be engineered to express TRIM21 either chromosomally or by using a vectored delivery of a sequence encoding TRIM21.

As used herein, the term “chimeric antigen receptor (CAR)” refers to a synthetic receptor that mimics the TCR on the surface of T cells. In general, a CAR is composed of an extracellular targeting domain, a transmembrane domain/region and an intracellular signaling/activation domain. In a standard CAR receptor, the components: the extracellular targeting domain, transmembrane domain and intracellular signaling/activation domain, are linearly constructed as a single fusion protein. The extracellular region comprises a targeting domain/moiety (e.g., a scFv) that recognizes a specific tumor antigen or other tumor cell-surface molecules. The intracellular region may contain a signaling domain of TCR. complex (e.g., the signal region of CD3C), and/or one or more costimulatory signaling domains, such as those from CD28, 4-1BB (CD137) and OX-40 (CD134). For example, a “first-generation CAR” only has the CD3 signaling domain, whereas, a second-generation CARs have a CD3ζ signal domain plus one costimulatory signaling domain, and a third-generation CARs having CD3ζ signal domain plus two or more costimulatory signaling domains. A CAR, when expressed by a T cell, endows the T cell with antigen specificity determined by the extracellular targeting moiety of the CAR. It is also desirable to add one or more elements such as homing and suicide genes to develop a more competent and safer architecture of CAR, which has given rise to the so called the fourth-generation CAR.

In some embodiments, the extracellular targeting domain is joined through the hinge (also called space domain or spacer) and transmembrane regions to an intracellular signaling domain. The hinge may need to be varied to optimize the potency of CAR expressing cells towards the cancer cells due to the size of the target protein where the targeting moiety binds, and the size and affinity of the targeting domain itself. Upon recognition and binding of the targeting moiety to the target cell, the intracellular signaling domain leads to an activation signal for the CAR T cell, which is further amplified by the “second signal” from one or more intracellular costimulatory domains. The CAR T cell, once activated, can destroy the target cell.

In some embodiments, the CAR of the present disclosure may be split into two parts, each part is linked a dimerizing domain, such that an input that triggers the dimerization promotes assembly of the intact functional receptor. Wu and Lim recently reported a split CAR in which the extracellular CD19 binding domain and the intracellular signaling element are separated and linked to the FKBP domain and the FRB* (T2089L mutant of FKBP-rapamycin binding) domain that heterodimerize in the presence of the rapamycin analog AP2.1967. The split receptor is assembled in the presence of AP21967 and together with the specific antigen binding, activates T cells (Wu et al, Science, 2015, 625(6258): aab4077).

In accordance with the disclosure, the extracellular target moiety of a CAR may be any agent that recognizes and binds to a given target molecule, for example, a neoantigen on tumor cells, with high specificity and affinity. The target moiety may be an antibody and variants thereof that specifically bind to a target molecule on tumor cells, or a peptide aptamer selected from a random sequence pool based on its ability to bind to the target molecule on tumor cells, or a variant or fragment thereof that can bind to the target molecule on tumor cells, or an antigen recognition domain from native T-cell receptor (TCR) (e.g. CD4 extracellular domain to recognize HIV infected cells), or exotic recognition components such as a linked cytokine that leads to recognition of target cells bearing the cytokine receptor, or a natural ligand of a receptor.

In some embodiments, the targeting domain of a CAR may be a Ig NAR, a Fab fragment, a Fab′ fragment, a F(ab)′2 fragment, a F(ab)′3 fragment, Fv, a single chain variable fragment (scFv), a bis-scFv, a (scFv)2, a minibody, a diabody, a triabody, a tetrabody, a disulfide stabilized Fv protein (dsFv), a unibody, a nanobody, or an antigen binding region derived from an antibody that specifically recognizes a target molecule, for example a tumor specific antigen (TSA). In some embodiments, the targeting moiety is a scFv antibody. The scFv domain, when it is expressed on the surface of a CAR T cell and subsequently binds to a target protein on a cancer cell, is able to maintain the CAR T cell in proximity to the cancer cell and to trigger the activation of the T cell. A scFv can be generated using routine recombinant DNAA_technology techniques and is discussed in the present disclosure.

In some embodiments, the targeting moiety of a CAR construct may be a natural ligand of the target molecule, or a variant and/or fragment thereof capable of binding the target molecule. In some aspects, the targeting moiety of a CAR may be a receptor of the target molecule, for example, a full length human CD27, as a CD70 receptor, may be fused in frame to the signaling domain of CD3ζ forming a CD27 chimeric receptor as an immunotherapeutic agent for CD70-positive malignancies (see, e.g., US patent publication NO: US20130323214; the contents of which are incorporated by reference herein in their entirety).

In some embodiments, the targeting moiety of a CAR may recognize a tumor specific antigen (TSA), for example a cancer neoantigen whose expression is restricted to tumor cells.

As non-limiting examples, the CAR of the present disclosure may comprise the extracellular targeting domain capable of binding to a tumor specific antigen selected from 5T4, 707-AP, A33, AFP (α-fetoprotein), AKAP-4 (A kinase anchor protein 4), ALK, α5β1-integrin, androgen receptor, annexin II, alpha-actinin-4, ART-4, B1, B7113, B7114, BAGE (B melanoma antigen), BCMA, BCR-ABL fusion protein, beta-catenin, BKT-antigen, BTAA, CA-I (carbonic anhydrase I), CA50 (cancer antigen 50), CA125, CA15-3, CA195, CA242, calretinin, CAIX (carbonic anhydrase), CAMEL (cytotoxic T-lymphocyte recognized antigen on melanoma), CAM43, CAP-1, Caspase-8/m, CD4, CD5, CD7, CD19, CD20, CD22, CD23, CD25, CD27/m, CD28, CD30, CD33, CD34, CD36, CD38, CD40/CD154, CD41, CD44v6, CD44v7/8, CD45, CD49f, CD56, CD68KP1, CD74, CD79a/CD79b, CD103, CD123, CD133, CD138, CD171, cdc27/m, CDK4 (cyclin dependent kinase 4), CDKN2A, CD5, CEA (carcinoembryonic antigen), CEACAMS, CEACAM6, chromogranin, c-Met, c-Myc, coa-1, CSAp, CT7, CT10, cyclophilin B, cyclin B1, cytoplasmic tyrosine kinases, cytokeratin, DAM-10, DAM-6, dek-can fusion protein, desmin, DEPDC1 (DEP domain containing 1), E2A-PRL, EBNA, EGF-R (epidermal growth factor receptor), EGP-1(epithelial glycoprotein-1) (TROP-2), EGP-2, EGP-40, EGFR (epidermal growth factor receptor), EGFRvIII, EF-2, ELF2M, EMMPRIN, EpCAM (epithelial cell adhesion molecule), EphA2, Epstein Barr virus antigens, Erb (ErbB1; ErbB3; ErbB4), ETA (epithelial tumor antigen), ETV6-AML1 fusion protein, FAP (fibroblast activation protein), FBP (folate-binding protein), FGF-5, folate receptor α, FOS related antigen 1, fucosyl GM1, G250, GAGE (GAGE-1; GAGE-2), galactin, GD2 (ganglioside), GD3, GFAP (glial fibrillary acidic protein), GM2 (oncofetal antigen-immunogenic-1; OFA-I-1), GnT-V, Gp100, H4-RET, HAGE (helicase antigen), HER-2/neu, HIFs (hypoxia inducible factors), HIF-1α, HIF-2α, HLA-A2, HLA-A*0201-R1701, HLA-A11, HMWMAA, Hom/Mel-40, HSP70-2M (Heat shock protein 70), HST-2, HTgp-175, hTERT (or hTRT), human papillomavirus-E6/human papillomavirus-E7 and E6, iCE (immune-capture EIA), IGH-IGK, IL-2R, IL-5, ILK (integrin-linked kinase), IMP3 (insulin-like growth factor II mRNA-binding protein 3), IRF4 (interferon regulatory factor 4), KDR (kinase insert domain receptor), KIAA0205, KRAB-zinc finger protein (KID)-3; KID31, KSA (17-1A), K-ras, LADE, LCK, LDLR/FUT (LDLR-fucosyltransferaseAS fusion protein), LeY (Lewis Y), MAD-CT-1, MAGE (tyrosinase, melanoma-associated antigen) (MAGE-1; MAGE-3), melan-A tumor antigen (MART), MART-2/Ski, MC1R (melanocortin 1 receptor), MDM2, mesothelin, MPHOSPH1, MSA(muscle-specific actin), mTOR (mammalian targets of rapamycin), MUC-1, MUC-2, MUM-1 (melanoma. associated antigen (mutated) 1), MUM-2, MUM-3, Myosin/m, MYL-RAR, NA88-A, N-acetylglucosaminyltransferase, neo-PAP, NF-KB (nuclear factor-kappa B), neurofilament, NSE (neuron-specific enolase), Notch receptors, NuMa, N-Ras, NY-BR-1, NY-CO-1, NY-ESO-1, Oncostatin M, OS-9, OY-TES1, pS3 mutants, p190 minor bcr-abl, p15(58), p185erbB2, p180erbB-3; PAGE (prostate associated gene), PAP (prostatic acid phosphatase), PAX3, PARS, PDGFR (platelet derived growth factor receptor), cytochrome P450 involved in piperidine and pyrrolidine utilization (MA); Pml-RAR alpha fusion protein, PR-3 (proteinase 3), PSA (prostate specific antigen), PSM, PSMA (Prostate stem cell antigen), PRAME (preferentially expressed antigen of melanoma), PTPRK, RAGE (renal tumor antigen), Raf (A-Raf, B-Raf and C-Raf), Ras, receptor tyrosine kinases, RCAS1, RGSS, RORI (receptor tyrosine kinase-like orphan receptor 1), RU1, RU2, SAGE, SART-1, SART-3, SCP-1, SDCCAG16, SP-17 (sperm protein 17), src-family, SSX (synovial sarcoma X breakpoint)-1, SSX-2(HOM-MEL-40), SSX-3, SSX-4, STAT-3, STAT-5, STAT-6, STEAD, STn, survivin, syk-ZAP70, TA-90 (Mac-2 binding protein\cyclophilin C-associated protein), TAAL6, TACSTD1 (tumor associated calcium signal transducer 1), TACSTD2, TAG-72-4, TAGE, TARP (T cell receptor gamma alternate reading frame protein), TEL/AML1 fusion protein, TEM1, TEM8 (endosialin or CD248), TGFβ, T1F2, TLP, IMERSS2 ETS fusion gene, TNF-receptor (TNF-α, receptor, TNF-β receptor; or TNF-γ receptor), transferrin receptor, TPS, TRP-1 (tyrosine related protein 1), TRP-2, TRP-2/INT2, TSP-180, VEGF receptor, WNT, WT-1 (Wilm's tumor antigen) and XAGE.

As non-limiting examples, the targeting moiety of the present disclosure may be a scFv antibody that recognizes a tumor specific antigen (TSA), for example scFvs of antibodies SS, SS1 and HN1 that specifically recognize and bind to human mesothelin (U.S. Pat. No. 9,359,447), scFv of antibody of GD2 (U.S. Pat. No. 9,315,585), a CD19 antigen binding domain (U.S. Pat. No. 9,328,156); a NKG2D ligand binding domain (U.S. Pat. No. 9,273,283; US patent publication NO.: US20160311906A1); human anti-mesothelin scFvs comprising the amino acid sequences of SEQ ID NO.: 11 and 12 of U.S. Pat. No. 9,272,002; an anti-CS1 binding agent (US patent publication NO.: 11520160075784); an anti-BCMA binding domain (International Patent Publication NO. WO2016/014565); anti-CD1.9 scFv antibody of SEQ ID NO.: 20 in U.S. Pat. No. 9,102,761; GFR alpha 4 antigen binding fragments having the amino acid sequences of SEQ ID NOs.: 59 and 79 of International patent publication NO.: 2016/025880; anti-CLL-1 (C-type lectin-like molecule 1) binding domains having the amino acid sequences of SEQ ID NO.:47, 44, 48, 49, 50, 39, 40, 41, 42, 43, 45, 46, 51, 73, 70, 74, 75, 76, 65, 66, 67, 68, 69, 71, 72, 77, 195, 86, 83, 87, 88, 89, 78, 79, 80, 81, 82, 84, 85, 90 and 196 of International Patent Publication NO.: WO2016014535); CD33 binding domains having the amino acid sequences of SEQ ID NOs.: 39-46 of International patent publication NO.: WO2016014576; a GPC3 (glypican-3) binding domain (SEQ ID NO.: 2 and SEQ ID NO.: 4 of International patent publication NO.: WO2016036973), a GFR alpha4 (Glycosyl-phosphatidylinositol (GPI)-linked GDNF family a receptor 4 cell-surface receptor) binding domain (International Patent Publication NO.: WO2016025880); CD123 binding domains having the amino acid sequences of SEQ ID NOs.: 480, 483, 485, 478, 158, 159, 160, 157, 217, 218, 219, 216, 276, 277, 278, and 275 of International patent publication NO.: WO20160258896; an anti-RORI antibody or fragments thereof (International patent publication NO.: WO2016016344); says specific to GPC-3 (SEQ ID NOs.: 1 and 24 of International patent publication NO.: WO2016049459); scFv for CSPG4 (SEQ ID NO.: 2 of International patent publication NO.: NV02015080981; scFv for folate receptor alpha (US Patent Publication NO.: US20170002072A1); the contents of each of which are incorporated herein by reference in their entirety.

The intracellular domain of a CAR fusion polypeptide, after binding to its target molecule, transmits a signal to the immune effector cell, activating at least one of the normal effector functions of immune effector cells, including cytolytic activity (e.g., cytokine secretion) or helper activity. Therefore, the intracellular domain comprises an “intracellular signaling domain” of a T cell receptor (TCR). In some embodiments, the intracellular signaling domain of the present disclosure may contain signaling motifs which are known as immunoreceptor tyrosine-based activation motifs (ITAMs). In some embodiments, the intracellular region of the present disclosure further comprises one or more costimulatory signaling domains which provide additional signals to the immune effector cells. These costimulatory signaling domains, in combination with the signaling domain can further improve expansion, activation, memory, persistence, and tumor-eradicating efficiency of CAR engineered immune cells (e.g., CAR T cells). In some cases, the costimulatory signaling region contains 1, 2, 3, or 4 cytoplasmic domains of one or more intracellular signaling and/or costimulatory molecules. In some embodiments, the intracellular region of the present disclosure may comprise a functional signaling domain from a protein selected from the group consisting of an MHC class I molecule, a TNF receptor protein, an immunoglobulin-like protein, a cytokine receptor, an integrin, a signaling lymphocytic activation protein (SLAM) such as CD48, CD229, 2B4, CD84, NTB-A, CRACC, BLAME, CD2F-10, SLAMF6, SLAMF7, an activating NK cell receptor, BTLA, a Toll ligand receptor, 0X40, CD2, CD7, CD27, CD28, CD30, CD40, CD5, ICAM-1, LFA-1 (CM11a/CD18), 4-1BB (CD137), B7-H3, CDS, ICAM-1, ICOS (CD278), GITR, BAFFR, LIGHT, HVEM (LIGHTR), SLAMF7, NKp80 (KLRF1), NKp44, NKp30, NKp46, CD19, CD4, CD8alpha, CD8beta, IL2R beta, IL2R gamma, IL7R alpha, IL-15Ra, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, NKD2, CSLP76, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, CD270 (MEM), GADS, SLP-76, PAG/Cbp, CD19a, a ligand that specifically binds with CD83, DAP 10, TRIM, ZAP70, Killer immunoglobulin receptors (KIRs) such as KIR2IL1, KIR2DL2/L3, KIR2DL4, KIR2DL5A, KIR2DL5B, KIR2DS1, KIR2DS2, KIR2DS3, KIR2DS4, KIR2DS5, KIR3DL1/S1, KIR3DL2, KIR3DL3, and KIR2DP1; lectin related NK cell receptors such as Ly49, Ly49A, and. Ly49C.

In some embodiments, the CAR of the present disclosure may comprise a transmembrane domain. As used herein, the term “Transmembrane domain™” refers broadly to an amino acid sequence of about 15 residues in length which spans the plasma membrane. More preferably, a transmembrane domain includes at least 20, 21, 22, 23, 24, 25, 26, 27, 28, 29, 30, 31, 32, 33, 34, 35, 36, 37, 38, 39, 40, 41, 42, 43, 44, or 45 amino acid residues and spans the plasma membrane. In some embodiments, the transmembrane domain of the present disclosure may be derived either from a natural or from a synthetic source. The transmembrane domain of a CAR may be derived from any naturally membrane-bound or transmembrane protein. In some embodiments, the transmembrane domain of the present disclosure may be selected from the group consisting of a CD8a transmembrane domain, a CD4 transmembrane domain, a CD 28 transmembrane domain, a CTLA-4 transmembrane domain, a PD-1 transmembrane domain, and a human IgG4 Fc region.

In some embodiments, the CAR of the present disclosure may comprise an optional hinge region (also called spacer). A hinge sequence is a short sequence of amino acids that facilitates flexibility of the extracellular targeting domain that moves the target binding domain away from the effector cell surface to enable proper cell/cell contact, target binding and effector cell activation (Patel et al., Gene Therapy, 1999; 6: 412-419). The hinge sequence may be positioned between the targeting moiety and the transmembrane domain. The hinge sequence can be any suitable sequence derived or obtained from any suitable molecule. The hinge sequence may be derived from all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge, the extracellular regions of type 1 membrane proteins such as CD8a. CD4, CD28 and CD7, which may be a wild type sequence or a derivative.

### Disease Specific Epitopes, Innate Defense Regulator Peptides Cyclic Peptides

In some embodiments, the viral genomes of the viral particles may comprise nucleic acids which have been engineered to enable expression of antibodies binding to disease-specific epitopes of proteins. Such antibodies may be used to diagnose, prevent, and/or treat the corresponding medical conditions by targeting epitopes of the protein presented by or accessible on native or non-native forms (e.g., misfolded forms of native proteins) of the target. Such epitopes may be specific to diseases involved with misfolding of a protein due to pathologic condition and resulting in misfolded aggregates. The disease-specific proteins are considered to be toxic to neurons and to have a role in neuronal cell death and dysfunction in neurodegenerative diseases including, but not limited to, Alzheimer's disease (AD), amyotrophic lateral sclerosis (ALS), Parkinson's disease, dementia by Lewy body (DLB), and prion diseases, e.g. Creutzfeldt-Jakob disease (CJD), Gerstmann-Straussler-Scheinker syndrome (GSS), kuru, and fatal familial insomnia (FFI). By also utilizing a vectored TRI 21 construct, such antibody destruction, expression or regulation may be augmented as with the present VA-DER systems.

In some embodiments, the encoded disease-specific epitopes may include epitopes on SOD1 that are revealed as SOD1 (Superoxide dismutase [Cu—Zn]) dissociates from its homodimeric, normal state. The SOD epitopes may be selectively presented or accessible in non-native SOD1 forms including misfolded SOD1 monomer, misfolded SOD1 dimer, and the epitopes selectively presented or accessible in SOD1 aggregates. Such epitopes may be specific to neurodegenerative diseases including, but not limited to, amyotrophic lateral sclerosis (ALS), Alzheimer's (AD), Parkinson's (PD), and Lewy body diseases (LBD).

In some embodiments, the expressed antibodies may bind to epitopes presented by or accessible on non-native forms of SOD1, such as those presented by SEQ ID NO: 2, 3, 5, 6, and 7 of U.S. Pat. No. 7,977,314 (the contents of which are herein incorporated by reference in its entirety), or presented by or accessible on monomeric forms of SOD1, such as those presented by SEQ ID NO: 1 and 4 of U.S. Pat. No. 7,977,314, the contents of which are herein incorporated by reference in their entirety. In some embodiments, the expressed antibodies may comprise isolated peptides corresponding to such epitopes, such as those presented in SEQ ID NO: 1-8 or SEQ ID NO: 8-16, or epitopes presented by SEQ ID N0: 34-63, 65-79 of U.S. Pat. No. 7,977,314, the contents of which are herein incorporated by reference in their entirety.

In some embodiments, the encoded disease-specific epitopes may be specific to diseases associated with prion protein (PrP); familial amyloid polyneuropathy or senile systemic amyloidosis or a disease related by the presence of misfolded transthyretine (TTR); renal accumulation of 132 microglobulin amyloid deposits or a disease related by the presence of misfolded 132 microglobulin, amyotrophic lateral sclerosis (AI 0.5) or a disease related by the presence of misfolded SOD1; leukemias or myelomas or a disease related by the presence of misfolded cluster of differentiation 38 (CD38); colon cancer metastasis and or a disease related by the presence of misfolded cluster of differentiation (CD44); tumors associated with tumor necrosis factor receptor (TNFR); cancers including cervical, head and neck, endometrial, lung and breast carcinomas, pleural mesotheliomas, malignant melanomas, Hodgkin lymphomas, anaplastic large cell non-Hodgkin lymphomas, or a disease related by the presence of misfolded. Notch homolog 1 (NOTCH1) e.g. acute myeloid leukemias and B-cell chronic lymphoid leukemias; cancer in which Fas receptor (FasR) is implicated; cancers and related disorders in which misfolded epidermal growth factor (EGFR_) is implicated; and/or other related diseases, disorders and conditions.

In some embodiments, the encoded disease specific epitopes may include epitopes that are revealed as the proteins misfold. In some embodiments, the expressed antibodies may bind to predicted epitopes of human PrP, such as those presented by SEQ ID NO: 1-10 of US Patent Publication No. US20100233176; bovine PIT, such as those presented by SEQ ID NO: 11-15 of US Patent Publication No. US20100233176, TTR, such as those presented by SEQ ID NO: 16-22 of US Patent Publication No. US20100233176; beta-2 microglobulin, such as those presented by SEQ ID NO: 23-26 of US Patent Publication No. US20100233176; SOD1, such as those presented by SEQ ID NO: 27-40 of US Patent Publication No. US20100233176; CD38, such as those presented by SEQ ID NO: 41-45 of US Patent Publication No. US20100233176; CD44, such as those presented by 46-50 of US Patent Publication No. US20100233176; TNFR, such as those presented by 51-55 of US Patent Publication No. US20100233176; notch protein, such as those presented in SEQ ID NO: 56-60 of US Patent Publication No. US20100233176; FasR, such as those presented by SEQ ID NO: 61-65 of US Patent Publication No. US20100233176; and EGFR, such as those presented by SEQ ID NO: 66-80 of US Patent Publication No. US20100233176; the contents of which are herein incorporated by reference in their entirety.

In some embodiments, the expressed antibodies may comprise peptides corresponding to such epitopes. In some embodiments, the expressed antibodies may comprise prion-specific peptides, such as those presented by SEQ ID NO: 81-88 of US Patent Publication No. US20100233176, the contents of which are herein incorporated by reference in their entirety, and variations thereof.

In some embodiments, the encoded disease-specific epitopes may be specific to prion diseases, including transmissible spongiform encephalopathies (TSEs) or other prion diseases. In some embodiments, the expressed antibodies may bind to predicted epitopes of PrP, such as those presented by SEQ ID NO: 24, 26, 28, 30, 34, 36, 39-43, of US Patent Publication No. US20150004185, the contents of which are herein incorporated by reference in their entirety. In some embodiments, the expressed antibodies may comprise prion-specific peptides or peptide fusions, such as those presented by SEQ ID NO: 12-23, 25, 27, 29, 31, 33, 35, 37, 38, 43, and 44-48 of US Patent Publication No. US20150004185, the contents of which are herein incorporated by reference in their entirety.

In some embodiments, the expressed antibodies may comprise prion peptides binding to prion specific abnormal isoform of the prion protein, such as those presented by SEQ ID NO: 2-10 of US Patent Publication No. US20040072236, the contents of which are herein incorporated by reference in their entirety.

In some embodiments, the viral genomes of the viral particles may comprise nucleic acids which have been engineered to express innate defense regulator (IDR) peptides. IDRs are immunomodulatory peptides that act directly on cells to affect an innate immune response. Such IDRs may be used to treat neurodegenerative diseases associated with neuroinflammation, e.g. amyotrophic lateral sclerosis (ALS), Alzheimer's disease, Friedreich's ataxia, Huntington's disease, Lewy body disease, Parkinson's disease, spinal muscular atrophy, and multiple sclerosis (MS) and other neurodegenerative diseases. In some embodiments, IDRs may be those presented by SEQ ID NO: 1-969, and 973-1264 of International Publication No. WO2013034982, the contents of which are herein incorporated by reference in their entirety, or analogs, derivatives, amidated variations and conservative variations thereof.

In some embodiments, the viral genomes of the viral particles may comprise nucleic acids which have been engineered to express antibodies binding to an epitope of the Tropomyosin receptor kinase (TrkC) receptor. Such antibodies may comprise a peptide, such as one presented by SES) Il) NO: 1 of U.S. Pat. No. 9,200,080, the contents of which are herein incorporated by reference in their entirety.

In some embodiments, the viral genomes of the viral particles may comprise nucleic acids which have been engineered to express cyclic peptides with an amino acid sequence SNK. Non-limiting examples of other cyclic peptides include SEQ ID NO: 1-7 of U.S. Pat. No. 9,216,217, the contents of which are herein incorporated by reference in their entirety. The method of preparing the antibodies may include hyperimmune preparation method, as described in U.S. Pat. No. 9,216,217, the contents of which are herein incorporated by reference in their entirety.

### Prions

In some embodiments, the viral genomes of the viral particles may comprise a nucleic acid sequence encoding antibodies comprising prion peptides comprising prion epitopes, and fusions and repeats thereof, such as those presented by SEQ NO: 8-32, 35, and 36 of U.S. patent Ser. No. US/905,6918, the contents of which are herein incorporated by reference in their entirety.

In some embodiments, the viral genomes of the viral particles may comprise a nucleic acid sequence encoding prion binding proteins (PrPBP). In some embodiments, the PrPBPs are cadherins, such as those presented by SEQ ID NO: 1 and 2 of International Publication WO1997/045746, the contents of which are herein incorporated by reference in their entirety. In some embodiments, the PrPBPs are cadherins, such as those presented by SEQ ID NO: 2 and 7-9 of International Publication No. WO2001000235, the contents of which are herein incorporated by reference in their entirety.

### The Nature of the Polypeptides and Variants

Antibodies as well as any proteins, including TRIM21, encoded by payload regions of the viral genomes of the disclosure may be translated as a whole polypeptide, a plurality of polypeptides or fragments of polypeptides, which independently may be encoded by one or more nucleic acids, fragments of nucleic acids or variants of any of the aforementioned. As used herein, “polypeptide” means a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

In some embodiments “variant mimics” are provided. As used herein, the term “variant mimic” is one which contains one or more amino acids which would mimic an activated sequence. For example, glutamate may serve as a mimic for phosphoro-threonine and/or phosphoro-serine. Alternatively, variant mimics may result in deactivation or in an inactivated product containing the mimic, e.g., phenylalanine may act as an inactivating substitution for tyrosine; or alanine may act as an inactivating substitution for serine.

The term “amino acid sequence variant” refers to molecules with some differences in their amino acid sequences as compared to a native or starting sequence. The amino acid sequence variants may possess substitutions, deletions, and/or insertions at certain positions within the amino acid sequence. “Native” or “starting” sequence should not be confused with a wild type sequence. As used herein, a native or starting sequence is a relative term referring to an original molecule against which a comparison may be made. “Native” or “starting” sequences or molecules may represent the wild-type (that sequence found in nature) but do not have to be the wild-type sequence.

Ordinarily, variants will possess at least about 70?/(homology to a native sequence, and preferably, they will be at least about 80%, more preferably at least about 90% homologous to a native sequence. “Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to amino acid sequences is meant the corresponding sequence of other species having substantial identity to a second sequence of a second species.

“Analogs” is meant to include polypeptide variants which differ by one or more amino acid alterations, e.g., substitutions, additions, or deletions of amino acid residues that still maintain the properties of the parent polypeptide.

Sequence tags or amino acids, such as one or more lysines, can be added to the peptide sequences of the disclosure (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble or linked to a solid support.

“Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to proteins are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants” when referring to proteins, are those with one or more amino acids in the native or starting amino acid sequence removed. Ordinarily, deletional variants will have one or more amino acids deleted in a particular region of the molecule.

As used herein, the term “derivative” is used synonymously with the term “variant” and refers to a molecule that has been modified or changed in any way relative to a reference molecule or starting molecule. In some embodiments, derivatives include native or starting proteins that have been modified with an organic proteinaceous or non-proteinaceous derivatizing agent, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present disclosure.

Other post-translational modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W. H. Freeman & Co., San Francisco, pp. 79-86 (1983)).

“Features” when referring to proteins are defined as distinct amino acid sequence-based components of a molecule. Features of the proteins of the present disclosure include surface manifestations, local conformational shape, folds, loops, half-loops, domains, half-domains, sites, termini or any combination thereof.

As used herein when referring to proteins the term “surface manifestation” refers to a polypeptide-based component of a protein appearing on an outermost surface.

As used herein when referring to proteins the term “local conformational shape” means a polypeptide based structural manifestation of a protein which is located within a definable space of the protein.

As used herein when referring to proteins the term “fold” means the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein when referring to proteins the term “loop” refers to a structural feature of a peptide or polypeptide which reverses the direction of the backbone of a peptide or polypeptide and comprises four or more amino acid residues. Oliva et al. have identified at least 5 classes of protein loops (J. Mol Biol 266 (4): 814-830; 1997).

As used herein when referring to proteins the term “half-loop” refers to a portion of an identified loop having at least half the number of amino acid residues as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7-amino acid loop could produce half-loops of 3 amino acids or 4 amino acids (7/2=3.5+/−0.5 being 3 or 4).

As used herein when referring to proteins the term “domain” refers to a motif of a polypeptide having one or more identifiable structural or functional characteristics or properties (e.g., binding capacity, serving as a site for protein-protein interactions).

As used herein when referring to proteins the term “half-domain” means portion of an identified domain having at least half the number of amino acid residues as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7-amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein when referring to proteins the terms “site” as it pertains to amino acid based embodiments is used synonymous with “amino acid residue” and “amino acid side chain”. A site represents a position within a peptide or polypeptide that may be modified, manipulated, altered, derivatized or varied within the polypeptide based molecules of the present disclosure.

As used herein the terms “termini or terminus” when referring to proteins refers to an extremity of a peptide or polypeptide. Such extremity is not limited only to the first or final site of the peptide or polypeptide but may include additional amino acids in the terminal regions. The polypeptide based molecules of the present disclosure may be characterized as having both an N-terminus (terminated by an amino acid with a free amino group (NH2)) and a C-terminus (terminated by an amino acid with a free carboxyl group (COOH)). Proteins of the disclosure are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide-based moiety such as an organic conjugate.

Once any of the features have been identified or defined as a component of a molecule of the disclosure, any of several manipulations and/or modifications of these features may be performed by moving, swapping, inverting, deleting, randomizing, or duplicating. Furthermore, it is understood that manipulation of features may result in the same outcome as a modification to the molecules of the disclosure. For example, a manipulation which involves deleting a domain would result in the alteration of the length of a molecule just as modification of a nucleic acid to encode less than a full-length molecule would.

Modifications and manipulations can be accomplished by methods known in the art such as site directed mutagenesis. The resulting modified molecules may then be tested for activity using in vitro or in vivo assays such as those described herein or any other suitable screening assay known in the art.

microRNA

microRNAs (or miRNA) are 19-25 nucleotide long noncoding RNAs that bind to the 3′ UTR. of nucleic acid molecules and down-regulate gene expression either by reducing nucleic acid molecule stability or by inhibiting translation. The polynucleotides of the disclosure may comprise one or more microRNA target sequences, microRNA sequences, or microRNA seeds. Such sequences may correspond to any known microRNA such as those taught in US Publication US2005/0261218 and US Publication US2005/0059005, the contents of which are incorporated herein by reference in their entirety. As a non-limiting embodiment, known microRNAs, their sequences and their binding site sequences in the human genome are listed below in Table 57.

A microRNA sequence comprises a “seed” region, i.e., a sequence in the region of positions 2-8 of the mature microRNA, which sequence has perfect Watson-Crick complementarity to the miRNA target sequence. A microRNA seed may comprise positions 2-8 or 2-7 of the mature microRNA. In some embodiments, a microRNA seed may comprise 7 nucleotides (e.g., nucleotides 2-8 of the mature microRNA), wherein the seed-complementary site in the corresponding miRNA target is flanked by an adenine (A) opposed to microRNA position 1. In some embodiments, a microRNA seed may comprise 6 nucleotides (e.g., nucleotides 2-7 of the mature microRNA), wherein the seed-complementary site in the corresponding miRNA target is flanked by an adenine (A) opposed to microRNA position 1. See for example, Grimson A, Farh K K, Johnston W K, Garrett-Engele P, Lim L P, Bartel D P; Mol Cell. 2007 Jul. 6; 27(1):91-105, The bases of the microRNA seed have complete complementarily with the target sequence. By engineering microRNA target sequences into the payloads of the disclosure one can target the molecule, provided the microRNA in question is available. This process will reduce the hazard of off target effects upon nucleic acid delivery.

Identification of microRNA, microRNA target regions, and their expression patterns and role in biology have been reported (Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is herein incorporated by reference in its entirety).

For example, if the polynucleotide is not intended to be delivered to the liver but ends up there, then miR-122, a microRNA abundant in liver, can inhibit the expression of the polynucleotide if one or multiple target sites of miR-122 are engineered into the polynucleotide. Introduction of one or multiple binding sites for different microRNA can be engineered to further decrease the longevity, stability, and protein translation of a polynucleotide.

As used herein, the term “microRNA. site” refers to a microRNA target site or a microRNA recognition site, or any nucleotide sequence to which a microRNA binds or associates. It should be understood that “binding” may follow traditional Watson-Crick hybridization rules or may reflect any stable association of the microRNA with the target sequence at or adjacent to the microRNA site.

Conversely, for the purposes of the polynucleotides of the present disclosure, microRNA binding sites can be engineered out of (i.e. removed from) sequences in which they naturally occur in order to increase protein expression in specific tissues. For example, miR-122 binding sites may be removed to improve protein expression in the liver.

Regulation of expression in multiple tissues can be accomplished through introduction or removal or one or several microRNA binding sites.

Specifically, microRNAs are known to be differentially expressed in immune cells (also called hematopoietic cells), such as antigen presenting cells (APCs) (e.g. dendritic cells and macrophages), macrophages, monocytes, B lymphocytes, T lymphocytes, granulocytes, natural killer cells, etc. Immune cell specific microRNAs are involved in immunogenicity, autoimmunity, the immune-response to infection, inflammation, as well as unwanted immune response after gene therapy and tissue/organ transplantation. Immune cells specific microRNAs also regulate many aspects of development, proliferation, differentiation and apoptosis of hematopoietic cells (immune cells). For example, miR-142 and miR-146 are exclusively expressed in the immune cells, particularly abundant in myeloid dendritic cells. Introducing the miR-142 binding site into the 3′-UTR of a polypeptide of the present disclosure can selectively suppress the gene expression in the antigen presenting cells through miR-142 mediated mRNA degradation, limiting antigen presentation in professional APCs (e.g. dendritic cells) and thereby preventing antigen-mediated immune response after gene delivery (see, Annoni A et al., blood, 2009, 114, 5152-5161, the content of which is herein incorporated by reference in its entirety.)

In some embodiments, microRNAs binding sites that are known to be expressed in immune cells, in particular, the antigen presenting cells, can be engineered into the polynucleotides to suppress the expression of the polynucleotide in APCs through microRNA mediated RNA degradation, subduing the antigen-mediated immune response, while the expression of the polynucleotide is maintained in non-immune cells where the immune cell specific microRNAs are not expressed.

Many microRNA expression studies have been conducted, and are described in the art, to profile the differential expression of microRNAs in various cancer cells/tissues and other diseases. Some microRNAs are abnormally over-expressed in certain cancer cells and others are under-expressed. For example, microRNAs are differentially expressed in cancer cells (WO2008/154098, US2013/0059015, US2013/0042333, WO2011/157294); cancer stem cells (US2012/0053224); pancreatic cancers and diseases (US2009/0131348, US2011/0171646, US2010/0286232, U.S. Pat. No. 8,389,210); asthma and inflammation (U.S. Pat. No. 8,415,096); prostate cancer (US2013/0053264); hepatocellular carcinoma (WO2012/151212, US2012/0329672, WO2008/054828, U.S. Pat. No. 8,252,538); lung cancer cells (WO2011/076143, WO2013/033640, WO2009/070653, US2010/0323357); cutaneous T cell lymphoma (WO2013/011378); colorectal cancer cells (WO2011/0281756, WO2011/076142); cancer positive lymph nodes (WO2009/100430; US2009/0263803); nasopharyngeal carcinoma (EP2112235); chronic obstructive pulmonary disease (US2012/0264626, US2013/0053263); thyroid cancer (WO2013/066678); ovarian cancer cells (US2012/0309645, WO2011/095623); breast cancer cells (WO2008/154098, WO2007/081740, US2012/0214699), leukemia and lymphoma (WO2008/073915; US2009/0092974, US2012/0316081, US2012/0283310, WO2010/018563, the content of each of which is incorporated herein by reference in their entirety).

TABLE 57
microRNA Sequences

| | | | | miR | |
|---|---|---|---|---|---|
| | | miR | | BS | |
| microRNA | | SEQ | | SEQ | Types of Tissues |
| name | miR SEQ | ID | miR BS SEQ | ID | and/or Cells |
| hsa-miR- | CCGGUUCCAGUCC | 28586 | CUCCAGGGACUGGA | 30628 | A colorectal |
| 6070 | CUGGAG | | ACCGG | | microRNAome |
| hsa-miR- | UGCUCAGGUUGC | 28587 | UCCCAGCUGUGCAA | 30629 | Abnormal skin |
| 3934-3p | ACAGCUGGGA | | CCUGAGCA | | (psoriasis) |
| hsa-miR- | UCAGGUGUGGAA | 28588 | CUGCCUCAGUUUCC | 30630 | Abnormal skin |
| 3934-5p | ACUGAGGCAG | | ACACCUGA | | (psoriasis) |
| hsa-miR- | CAAAACCGCGAUU | 28589 | UGCAAGAGUAAUCG | 30631 | Abnormal skin |
| 548ay-3p | ACUCUUGCA | | CGGUUUUG | | (psoriasis) |
| hsa-miR- | AAAAGUAAUUGU | 28590 | GCAAAAACCACAAU | 30632 | Abnormal skin |
| 548ay-5p | GGUUUUUGC | | UACUUUU | | (psoriasis) |
| hsa-miR- | AAAAACUGCAAU | 28591 | GCAAAAGUGAUUGC | 30633 | Abnormal skin |
| 548az-3p | CACUUUUGC | | AGUUUUU | | (psoriasis) |
| hsa-miR- | CAAAAGUGAUUG | 28592 | GCAAAAACCACAAU | 30634 | Abnormal skin |
| 548az-5p | UGGUUUUUGC | | CACUUUUG | | (psoriasis) |
| hsa-miR- | AGCAGUGUUUGU | 28593 | UGUGGGCAAAACAA | 30635 | Abnormal skin |
| 6499-3p | UUUGCCCACA | | ACACUGCU | | (psoriasis) |
| hsa-miR- | UCGGGCGCAAGA | 28594 | ACUGCAGUGCUCUU | 30636 | Abnormal skin |
| 6499-5p | GCACUGCAGU | | GCGCCCGA | | (psoriasis) |
| hsa-miR- | ACACUUGUUGGG | 28595 | GCAGGUCAUCCCAA | 30637 | Abnormal skin |
| 6500-3p | AUGACCUGC | | CAAGUGU | | (psoriasis) |
| hsa-miR- | AGGAGCUAUCCAC | 28596 | GGACACCUGGAGUG | 30638 | Abnormal skin |
| 6500-5p | UCCAGGUGUCC | | GAUAGCUCCU | | (psoriasis) |
| hsa-miR- | CCAGAGCAGCCUG | 28597 | ACUGUUACCGCAGG | 30639 | Abnormal skin |
| 6501-3p | CGGUAACAGU | | CUGCUCUGG | | (psoriasis) |
| hsa-miR- | AGUUGCCAGGGC | 28598 | ACCAAAGGCAGCCC | 30640 | Abnormal skin |
| 6501-5p | UGCCUUUGGU | | UGGCAACU | | (psoriasis) |
| hsa-miR- | UAGACCAUCUUUC | 28599 | AUACUCUAGAAAGA | 30641 | Abnormal skin |
| 6502-3p | UAGAGUAU | | UGGUCUA | | (psoriasis) |
| hsa-miR- | AGCUCUAGAAAG | 28600 | GGUCAACAAUCUUU | 30642 | Abnormal skin |
| 6502-5p | AUUGUUGACC | | CUAGAGCU | | (psoriasis) |
| hsa-miR- | GGGACUAGGAUG | 28601 | GGAGGUCUGCAUCC | 30643 | Abnormal skin |
| 6503-3p | CAGACCUCC | | UAGUCCC | | (psoriasis) |
| hsa-miR- | AGGUCUGCAUUC | 28602 | UCUGGGGAUUUGAA | 30644 | Abnormal skin |
| 6503-5p | AAAUCCCCAGA | | UGCAGACCU | | (psoriasis) |
| hsa-miR- | CAUUACAGCACAG | 28603 | AGAAUGGCUGUGCU | 30645 | Abnormal skin |
| 6504-3p | CCAUUCU | | GUAAUG | | (psoriasis) |
| hsa-miR- | UCUGGCUGUGCU | 28604 | CUGCAUUACAGCAC | 30646 | Abnormal skin |
| 6504-5p | GUAAUGCAG | | AGCCAGA | | (psoriasis) |
| hsa-miR- | UGACUUCUACCUC | 28605 | CUUUGGAAGAGGUA | 30647 | Abnormal skin |
| 6505-3p | UUCCAAAG | | GAAGUCA | | (psoriasis) |
| hsa-miR- | UUGGAAUAGGGG | 28606 | GCUGAGAUAUCCCC | 30648 | Abnormal skin |
| 6505-5p | AUAUCUCAGC | | UAUUCCAA | | (psoriasis) |
| hsa-miR- | UCGUAUCAGAGA | 28607 | GUGUCUGGAAUCUC | 30649 | Abnormal skin |
| 6506-3p | UUCCAGACAC | | UGAUACGA | | (psoriasis) |
| hsa-miR- | ACUGGGAUGUCA | 28608 | ACCAUAUUCAGUGA | 30650 | Abnormal skin |
| 6506-5p | CUGAAUAUGGU | | CAUCCCAGU | | (psoriasis) |
| hsa-miR- | CAAAGUCCUUCCU | 28609 | GGGAAAAAUAGGAA | 30651 | Abnormal skin |
| 6507-3p | AUUUUUCCC | | GGACUUUG | | (psoriasis) |
| hsa-miR- | GAAGAAUAGGAG | 28610 | ACAAAGUCCCUCCU | 30652 | Abnormal skin |
| 6507-5p | GGACUUUGU | | AUUCUUC | | (psoriasis) |
| hsa-miR- | UGGGCCAUGCAU | 28611 | AGUUCUAGAAAUGC | 30653 | Abnormal skin |
| 6508-3p | UUCUAGAACU | | AUGGCCCA | | (psoriasis) |
| hsa-miR- | UCUAGAAAUGCA | 28612 | GGUGGGUCAUGCAU | 30654 | Abnormal skin |
| 6508-5p | UGACCCACC | | UUCUAGA | | (psoriasis) |
| hsa-miR- | UUCCACUGCCACU | 28613 | AAAUUAGGUAGUGG | 30655 | Abnormal skin |
| 6509-3p | ACCUAAUUU | | CAGUGGAA | | (psoriasis) |
| hsa-miR- | AUUAGGUAGUGG | 28614 | GUUCCACUGCCACU | 30656 | Abnormal skin |
| 6509-5p | CAGUGGAAC | | ACCUAAU | | (psoriasis) |
| hsa-miR- | CACCGACUCUGUC | 28615 | CUGCAGGAGACAGA | 30657 | Abnormal skin |
| 6510-3p | UCCUGCAG | | GUCGGUG | | (psoriasis) |
| hsa-miR- | CAGCAGGGGAGA | 28616 | GACUCCUCUCUCUC | 30658 | Abnormal skin |
| 6510-5p | GAGAGGAGUC | | CCCUGCUG | | (psoriasis) |
| hsa-miR- | UUCCAGCCCUUCU | 28617 | CCUACCAUUAGAAG | 30659 | Abnormal skin |
| 6512-3p | AAUGGUAGG | | GGCUGGAA | | (psoriasis) |
| hsa-miR- | UACCAUUAGAAG | 28618 | UCUUCCAGCUCUUC | 30660 | Abnormal skin |
| 6512-5p | AGCUGGAAGA | | UAAUGGUA | | (psoriasis) |
| hsa-miR- | UCAAGUGUCAUC | 28619 | CUAGGGACAGAUGA | 30661 | Abnormal skin |
| 6513-3p | UGUCCCUAG | | CACUUGA | | (psoriasis) |
| hsa-miR- | UUUGGGAUUGAC | 28620 | AGACAUGUGGCGUC | 30662 | Abnormal skin |
| 6513-5p | GCCACAUGUCU | | AAUCCCAAA | | (psoriasis) |
| hsa-miR- | CUGCCUGUUCUUC | 28621 | CUGGAGUGGAAGAA | 30663 | Abnormal skin |
| 6514-3p | CACUCCAG | | CAGGCAG | | (psoriasis) |
| hsa-miR- | UAUGGAGUGGAC | 28622 | GCCAGCUGAAAGUC | 30664 | Abnormal skin |
| 6514-5p | UUUCAGCUGGC | | CACUCCAUA | | (psoriasis) |
| hsa-miR- | CCUCACCAUCCCU | 28623 | GCAGGCAGAAGGGA | 30665 | Abnormal skin |
| 6511a-3p | UCUGCCUGC | | UGGUGAGG | | (psoriasis) and |
| | | | | | epididymis |
| hsa-miR- | CAGGCAGAAGUG | 28624 | CCUGUCAGCCCCAC | 30666 | Abnormal skin |
| 6511a-5p | GGGCUGACAGG | | UUCUGCCUG | | (psoriasis) and |
| | | | | | epididymis |
| hsa-miR- | UCUCUUCAUCUAC | 28625 | CUGGGGGGUAGAUG | 30667 | Abnormal skin |
| 6515-3p | CCCCCAG | | AAGAGA | | (psoriasis) and |
| | | | | | epididymis |
| hsa-miR- | UUGGAGGGUGUG | 28626 | GAUGUCUUCCACAC | 30668 | Abnormal skin |
| 6515-5p | GAAGACAUC | | CCUCCAA | | (psoriasis) and |
| | | | | | epididymis |
| hsa-miR- | CUGCCAGCCCCGU | 28627 | UGCCCUGGAACGGG | 30669 | Acute Myeloid |
| 3972 | UCCAGGGCA | | GCUGGCAG | | Leukemia |
| hsa-miR- | ACAAAGUACAGC | 28628 | CUAAGGCUAAUGCU | 30670 | Acute Myeloid |
| 3973 | AUUAGCCUUAG | | GUACUUUGU | | Leukemia |
| hsa-miR- | AAAGGUCAUUGU | 28629 | GCAUUAACCUUACA | 30671 | Acute Myeloid |
| 3974 | AAGGUUAAUGC | | AUGACCUUU | | Leukemia |
| hsa-miR- | UGAGGCUAAUGC | 28630 | GUGAAGUAGUGCAU | 30672 | Acute Myeloid |
| 3975 | ACUACUUCAC | | UAGCCUCA | | Leukemia |
| hsa-miR- | UAUAGAGAGCAG | 28631 | ACAUUAAUCUUCCU | 30673 | Acute Myeloid |
| 3976 | GAAGAUUAAUGU | | GCUCUCUAUA | | Leukemia |
| hsa-miR- | GUGCUUCAUCGU | 28632 | UAAGGUUAAUUACG | 30674 | Acute Myeloid |
| 3977 | AAUUAACCUUA | | AUGAAGCAC | | Leukemia |
| hsa-miR- | GUGGAAAGCAUG | 28633 | ACACCCUGGAUGCA | 30675 | Acute Myeloid |
| 3978 | CAUCCAGGGUGU | | UGCUUUCCAC | | Leukemia |
| hsa-miR- | AGACACAUUUGG | 28634 | GGUUCCCUCUCCAA | 30676 | Adipocyte |
| 642a-3p | AGAGGGAACC | | AUGUGUCU | | |
| hsa-miR- | UUCCCUUUGUCAU | 28635 | AGGCAUAGGAUGAC | 30677 | Adipose, other |
| 204-5p | CCUAUGCCU | | AAAGGGAA | | tissues/cells, kidney |
| hsa-miR- | GCUGGGAAGGCA | 28636 | ACGUCCCUUUGCCU | 30678 | Adipose, other |
| 204-3p | AAGGGACGU | | UCCCAGC | | tissues/cells, |
| | | | | | Kidney |
| hsa-miR- | UACCACAGGGUA | 28637 | CCGUGGUUCUACCC | 30679 | Airway smooth |
| 140-3p | GAACCACGG | | UGUGGUA | | muscle |
| hsa-miR- | GGCUGGAGCGAG | 28638 | CACCACUGCACUCG | 30680 | B cells |
| 3135b | UGCAGUGGUG | | CUCCAGCC | | |
| hsa-miR- | CCAGGCUCUGCAG | 28639 | UCCCACUGCAGAGC | 30681 | B cells |
| 3155b | UGGGA | | CUGG | | |
| hsa-miR- | CUGGGAGGUGUG | 28640 | ACCACAAUAUCACA | 30682 | B cells |
| 3689c | AUAUUGUGGU | | CCUCCCAG | | |
| hsa-miR- | GGGAGGUGUGAU | 28641 | CGAGUGUGAGAUCA | 30683 | B cells |
| 3689d | CUCACACUCG | | CACCUCCC | | |
| hsa-miR- | UGUGAUAUCAUG | 28642 | UCCCAGGAACCAUG | 30684 | B cells |
| 3689e | GUUCCUGGGA | | AUAUCACA | | |
| hsa-miR- | UGUGAUAUCGUG | 28643 | UCCCAGGAAGCACG | 30685 | B cells |
| 3689f | CUUCCUGGGA | | AUAUCACA | | |
| hsa-miR- | GGUGGGCUUCCCG | 28644 | CCCUCCGGGAAGCC | 30686 | B cells |
| 4417 | GAGGG | | CACC | | |
| hsa-miR- | CACUGCAGGACUC | 28645 | CUGCUGAGUCCUGC | 30687 | B cells |
| 4418 | AGCAG | | AGUG | | |
| hsa-miR- | UGAGGGAGGAGA | 28646 | UGCAGUCUCCUCCC | 30688 | B cells |
| 1419a | CUGCA | | UCA | | |
| hsa-miR- | GAGGCUGAAGGA | 28647 | CCAUCUUCCUUCAG | 30689 | B cells |
| 4419b | AGAUGG | | CCUC | | |
| hsa-miR- | GUCACUGAUGUC | 28648 | CUCAGCUACAGACA | 30690 | B cells |
| 4420 | UGUAGCUGAG | | UCAGUGAC | | |
| hsa-miR- | ACCUGUCUGUGG | 28649 | UAGCUCCUUUCCAC | 30691 | B cells |
| 4421 | AAAGGAGCUA | | AGACAGGU | | |
| hsa-miR- | AGAGUUAACUCA | 28650 | UAGUCCAUUUUGAG | 30692 | B cells |
| 4424 | AAAUGGACUA | | UUAACUCU | | |
| hsa-miR- | UGUUGGGAUUCA | 28651 | AUGGUCCUGCUGAA | 30693 | B cells |
| 4425 | GCAGGACCAU | | UCCCAACA | | |
| hsa-miR- | GAAGAUGGACGU | 28652 | AAAGUACGUCCAUC | 30694 | B cells |
| 4426 | ACUUU | | UUC | | |
| hsa-miR- | UCUGAAUAGAGU | 28653 | ACUCUUCAGACUCU | 30695 | B cells |
| 4427 | CUGAAGAGU | | AUUCAGA | | |
| hsa-miR- | CAAGGAGACGGG | 28654 | GCUCCAUGUUCCCG | 30696 | B cells |
| 4428 | AACAUGGAGC | | UCUCCUUG | | |
| hsa-miR- | AAAAGCUGGGCU | 28655 | CGCCUCUCAGCCCA | 30697 | B cells |
| 4429 | GAGAGGCG | | GCUUUU | | |
| hsa-miR- | AGGCUGGAGUGA | 28656 | CUCCGCUCACUCCA | 30698 | B cells |
| 4430 | GCGGAG | | GCCU | | |
| hsa-miR- | GCGACUCUGAAA | 28657 | ACCUUCUAGUUUUC | 30699 | B cells |
| 4431 | ACUAGAAGGU | | AGAGUCGC | | |
| hsa-miR- | AAAGACUCUGCA | 28658 | AGGCAUCUUGCAGA | 30700 | B cells |
| 4432 | AGAUGCCU | | GUCUUU | | |
| hsa-miR- | ACAGGAGUGGGG | 28659 | AUGUCCCACCCCCA | 30701 | B cells |
| 4433-3p | GUGGGACAU | | CUCCUGU | | |
| hsa-miR- | CGUCCCACCCCCC | 28660 | ACAGGAGUGGGGGG | 30702 | B cells |
| 4433-5p | ACUCCUGU | | UGGGACG | | |
| hsa-miR- | AGGAGAAGUAAA | 28661 | UUCUACUUUACUUC | 30703 | B cells |
| 4434 | GUAGAA | | UCCU | | |
| hsa-miR- | AUGGCCAGAGCUC | 28662 | CCUCUGUGUGAGCU | 30704 | B cells |
| 4435 | ACACAGAGG | | CUGGCCAU | | |
| hsa-miR- | UGGGCUCAGGGU | 28663 | AACCUUUGUACCCU | 30705 | B cells |
| 4437 | ACAAAGGUU | | GAGCCCA | | |
| hsa-miR- | CACAGGCUUAGA | 28664 | ACUGUCUUUUCUAA | 30706 | B cells |
| 4438 | AAAGACAGU | | GCCUGUG | | |
| hsa-miR- | GUGACUGAUACC | 28665 | AUGCCUCCAAGGUA | 30707 | B cells |
| 4439 | UUGGAGGCAU | | UCAGUCAC | | |
| hsa-miR- | UGUCGUGGGGCU | 28666 | CAAGCCAGCAAGCC | 30708 | B cells |
| 4440 | UGCUGGCUUG | | CCACGACA | | |
| hsa-miR- | ACAGGGAGGAGA | 28667 | UACAAUCUCCUCCC | 30709 | B cells |
| 4441 | UUGUA | | UGU | | |
| hsa-miR- | GCCGGACAAGAG | 28668 | CCUCCCUCUUGUCC | 30710 | B cells |
| 4442 | GGAGG | | GGC | | |
| hsa-miR- | UUGGAGGCGUGG | 28669 | AAAACCCACGCCUC | 30711 | B cells |
| 4443 | GUUUU | | CAA | | |
| hsa-miR- | CUCGAGUUGGAA | 28670 | CGCCUCUUCCAACU | 30712 | B cells |
| 4444 | GAGGCG | | CGAG | | |
| hsa-miR- | CACGGCAAAAGA | 28671 | UGGAUUGUUUCUUU | 30713 | B cells |
| 4445-3p | AACAAUCCA | | UGCCGUG | | |
| hsa-miR- | AGAUUGUUUCUU | 28672 | UGCACGGCAAAAGA | 30714 | B cells |
| 4445-5p | UUGCCGUGCA | | AACAAUCU | | |
| hsa-miR- | GGUGGGGGCUGU | 28673 | AAACAACAGCCCCC | 30715 | B cells |
| 4447 | UGUUU | | ACC | | |
| hsa-miR- | GGCUCCUUGGUCU | 28674 | UACCCCUAGACCAA | 30716 | B cells |
| 4448 | AGGGGUA | | GGAGCC | | |
| hsa-miR- | CGUCCCGGGGCUG | 28675 | UGCCUCGCGCAGCC | 30717 | B cells |
| 4449 | CGCGAGGCA | | CCGGGACG | | |
| hsa-miR- | UGGGGAUUUGGA | 28676 | UCACCACUUCUCCA | 30718 | B cells |
| 4450 | GAAGUGGUGA | | AAUCCCCA | | |
| hsa-miR- | UGGUAGAGCUGA | 28677 | UGUCCUCAGCUCUA | 30719 | B cells |
| 4451 | GGACA | | CCA | | |
| hsa-miR- | UUGAAUUCUUGG | 28678 | AUCACUUAAGGCCA | 30720 | B cells |
| 4452 | CCUUAAGUGAU | | AGAAUUCAA | | |
| hsa-miR- | GAGCUUGGUCUG | 28679 | AACCGCUACAGACC | 30721 | B cells |
| 4453 | UAGCGGUU | | AAGCUC | | |
| hsa-miR- | GGAUCCGAGUCAC | 28680 | UGGUGCCGUGACUC | 30722 | B cells |
| 4454 | GGCACCA | | GGAUCC | | |
| hsa-miR- | AGGGUGUGUGUG | 28681 | AAAAACACACACAC | 30723 | B cells |
| 4455 | UUUUU | | CCU | | |
| hsa-miR- | CCUGGUGGCUUCC | 28682 | AAAAGGAAGCCACC | 30724 | B cells |
| 4456 | UUUU | | AGG | | |
| hsa-miR- | UCACAAGGUAUU | 28683 | UACGCCAGUCAAUA | 30725 | B cells |
| 4457 | GACUGGCGUA | | CCUUGUGA | | |
| hsa-miR- | AGAGGUAGGUGU | 28684 | UUCUUCCACACCUA | 30726 | B cells |
| 4458 | GGAAGAA | | CCUCU | | |
| hsa-miR- | CCAGGAGGCGGA | 28685 | CUCCACCUCCUCCG | 30727 | B cells |
| 4459 | GGAGGUGGAG | | CCUCCUGG | | |
| hsa-miR- | AUAGUGGUUGUG | 28686 | AAGGUAAAUUCACA | 30728 | B cells |
| 4460 | AAUUUACCUU | | ACCACUAU | | |
| hsa-miR- | GAUUGAGACUAG | 28687 | GCCUAGCCCUACUA | 30729 | B cells |
| 4461 | UAGGGCUAGGC | | GUCUCAAUC | | |
| hsa-miR- | UGACACGGAGGG | 28688 | UUCCCAAGCCACCC | 30730 | B cells |
| 4462 | UGGCUUGGGAA | | UCCGUGUCA | | |
| hsa-miR- | GAGACUGGGGUG | 28689 | GGCCCCACCCCAGU | 30731 | B cells |
| 4463 | GGGCC | | CUC | | |
| hsa-miR- | AAGGUUUGGAUA | 28690 | UAUUGCAUCUAUCC | 30732 | B cells |
| 4464 | GAUGCAAUA | | AAACCUU | | |
| hsa-miR- | CUCAAGUAGUCU | 28691 | UCCCCUGGUCAGAC | 30733 | B cells |
| 4465 | GACCAGGGGA | | UACUUGAG | | |
| hsa-miR- | GGGUGCGGGCCG | 28692 | CCCCGCCGGCCCGC | 30734 | B cells |
| 4466 | GCGGGG | | ACCC | | |
| hsa-miR- | AGAGCAGAAGGA | 28693 | AUCUCAUCCUUCUG | 30735 | B cells |
| 4468 | UGAGAU | | CUCU | | |
| hsa-miR- | UGGCAAACGUGG | 28694 | UCUCGGCUUCCACG | 30736 | B cells |
| 4470 | AAGCCGAGA | | UUUGCCA | | |
| hsa-miR- | GGUGGGGGGUGU | 28695 | AAAACAACACCCCC | 30737 | B cells |
| 4472 | UGUUUU | | CACC | | |
| hsa-miR- | CUAGUGCUCUCCG | 28696 | UACUUGUAACGGAG | 30738 | B cells |
| 4473 | UUACAAGUA | | AGCACUAG | | |
| hsa-miR- | CAAGGGACCAAGC | 28697 | AUAAUGAAUGCUUG | 30739 | B cells |
| 4475 | AUUCAUUAU | | GUCCCUUG | | |
| hsa-miR- | CAGGAAGGAUUU | 28698 | GCCUGUCCCUAAAU | 30740 | B cells |
| 4476 | AGGGACAGGC | | CCUUCCUG | | |
| hsa-miR- | CUAUUAAGGACA | 28699 | GAAUCACAAAUGUC | 30741 | B cells |
| 4477a | UUUGUGAUUC | | CUUAAUAG | | |
| hsa-miR- | AUUAAGGACAUU | 28700 | AUCAAUCACAAAUG | 30742 | B cells |
| 4477b | UGUGAUUGAU | | UCCUUAAU | | |
| hsa-miR- | GAGGCUGAGCUG | 28701 | CUCCUCAGCUCAGC | 30743 | B cells |
| 4478 | AGGAG | | CUC | | |
| hsa-miR- | CGCGCGGCCGUGC | 28702 | CUGCUCCGAGCACG | 30744 | B cells |
| 4479 | UCGGAGCAG | | GCCGCGCG | | |
| hsa-miR- | AGCCAAGUGGAA | 28703 | UAAAGUAACUUCCA | 30745 | B cells |
| 4480 | GUUACUUUA | | CUUGGCU | | |
| hsa-miR- | GGAGUGGGCUGG | 28704 | AACCACCAGCCCAC | 30746 | B cells |
| 1481 | UGGUU | | UCC | | |
| hsa-miR- | UUUCUAUUUCUC | 28705 | GAGCCCCACUGAGA | 30747 | B cells |
| 4482-3p | AGUGGGGCUC | | AAUAGAAA | | |
| hsa-miR- | AACCCAGUGGGCU | 28706 | CAUUUCCAUAGCCC | 30748 | B cells |
| 4482-5p | AUGGAAAUG | | ACUGGGUU | | |
| hsa-miR- | GGGGUGGUCUGU | 28707 | CAACAACAGACCAC | 30749 | B cells |
| 4483 | UGUUG | | CCC | | |
| hsa-miR- | AAAAGGCGGGAG | 28708 | UGGGGCUUCUCCCG | 30750 | B cells |
| 4484 | AAGCCCCA | | CCUUUU | | |
| hsa-miR- | UAACGGCCGCGGU | 28709 | UUAGGGUACCGCGG | 30751 | B cells |
| 4485 | ACCCUAA | | CCGUUA | | |
| hsa-miR- | GCUGGGCGAGGC | 28710 | UGCCAGCCUCGCCC | 30752 | B cells |
| 4486 | UGGCA | | AGC | | |
| hsa-miR- | AGAGCUGGCUGA | 28711 | CUGCCCUUCAGCCA | 30753 | B cells |
| 4487 | AGGGCAG | | GCUCU | | |
| hsa-miR- | AGGGGGGGGCU | 28712 | CGCCGGAGCCCGCC | 30754 | B cells |
| 4488 | CCGGCG | | CCCU | | |
| hsa-miR- | UCUGGUAAGAGA | 28713 | UAUGCCCAAAUCUC | 30755 | B cells |
| 4490 | UUUGGGCAUA | | UUACCAGA | | |
| hsa-miR- | AAUGUGGACUGG | 28714 | UUUGGUCACACCAG | 30756 | B cells |
| 4491 | UGUGACCAAA | | UCCACAUU | | |
| hsa-miR- | GGGGCUGGGCGC | 28715 | GGCGCGCGCCCAGC | 30757 | B cells |
| 4492 | GCGCC | | CCC | | |
| hsa-miR- | AGAAGGCCUUUCC | 28716 | ACAGAGAUGGAAAG | 30758 | B cells |
| 4493 | AUCUCUGU | | GCCUUCU | | |
| hsa-miR- | CCAGACUGUGGCU | 28717 | CCUCUGGUCAGCCA | 30759 | B cells |
| 4494 | GACCAGAGG | | CAGUCUGG | | |
| hsa-miR- | AAUGUAAACAGG | 28718 | AGCAAAAAGCCUGU | 30760 | B cells |
| 4495 | CUUUUUGCU | | UUACAUU | | |
| hsa-miR- | GAGGAAACUGAA | 28719 | CCCUCUCAGCUUCA | 30761 | B cells |
| 4496 | GCUGAGAGGG | | GUUUCCUC | | |
| hsa-miR- | CUCCGGGACGGCU | 28720 | GCCCAGCCGUCCCG | 30762 | B cells |
| 4497 | GGGC | | GAG | | |
| hsa-miR- | UGGGCUGGCAGG | 28721 | CAGCACUUGCCCUG | 30763 | B cells |
| 4498 | GCAAGUGCUG | | CCAGCCCA | | |
| hsa-miR- | AAGACUGAGAGG | 28722 | UCCCUCCUCUCAGU | 30764 | B cells |
| 4499 | AGGGA | | CUU | | |
| hsa-miR- | UGAGGUAGUAGU | 28723 | AAGAAACUACUACC | 30765 | B cells |
| 4500 | UUCUU | | UCA | | |
| hsa-miR- | UAUGUGACCUCG | 28724 | UGAUUCAUCCGAGG | 30766 | B cells |
| 4501 | GAUGAAUCA | | UCACAUA | | |
| hsa-miR- | GCUGAUGAUGAU | 28725 | CUUCAGCACCAUCA | 30767 | B cells |
| 4502 | GGUGCUGAAG | | UCAUCAGC | | |
| hsa-miR- | UUUAAGCAGGAA | 28726 | UAAAUUCUAUUUCC | 30768 | B cells |
| 4503 | AUAGAAUUUA | | UGCUUAAA | | |
| hsa-miR- | UGUGACAAUAGA | 28727 | CAUGUUCAUCUCUA | 30769 | B cells |
| 4504 | GAUGAACAUG | | UUGUCACA | | |
| hsa-miR- | AGGCUGGGCUGG | 28728 | UCCGUCCCAGCCCA | 30770 | B cells |
| 4505 | GACGGA | | GCCU | | |
| hsa-miR- | AAAUGGGUGGUC | 28729 | UUGCCUCAGACCAC | 30771 | B cells |
| 4506 | UGAGGCAA | | CCAUUU | | |
| hsa-miR- | CUGGGUUGGGCU | 28730 | CCCAGCCCAGCCCA | 30772 | B cells |
| 4507 | GGGCUGGG | | ACCCAG | | |
| hsa-miR- | GCGGGGCUGGGC | 28731 | CGCGCGCCCAGCCC | 30773 | B cells |
| 4508 | GCGCG | | CGC | | |
| hsa-miR- | ACUAAAGGAUAU | 28732 | AAAACCUUCUAUAU | 30774 | B cells |
| 4509 | AGAAGGUUUU | | CCUUUAGU | | |
| hsa-miR- | UGAGGGAGUAGG | 28733 | AACCAUACAUCCUA | 30775 | B cells |
| 4510 | AUGUAUGGUU | | CUCCCUCA | | |
| hsa-miR- | GAAGAACUGUUG | 28734 | AGGGCAAAUGCAAC | 30776 | B cells |
| 4511 | CAUUUGCCCU | | AGUUCUUC | | |
| hsa-miR- | CAGGGCCUCACUG | 28735 | UGGGCGAUACAGUG | 30777 | B cells |
| 4512 | UAUCGCCCA | | AGGCCCUG | | |
| hsa-miR- | AGACUGACGGCU | 28736 | AUGGGCCUCCAGCC | 30778 | B cells |
| 4513 | GGAGGCCCAU | | GUCAGUCU | | |
| hsa-miR- | ACAGGCAGGAUU | 28737 | UUCCCCAAUCCUGC | 30779 | B cells |
| 4514 | GGGGAA | | CUGU | | |
| hsa-miR- | AGGACUGGACUCC | 28738 | GGGCUGCCGGGAGU | 30780 | B cells |
| 4515 | CGGCAGCCC | | CCAGUCCU | | |
| hsa-miR- | GGGAGAAGGGUC | 28739 | GCCCCGACCCUUCU | 30781 | B cells |
| 4516 | GGGGC | | CCC | | |
| hsa-miR- | AAAUAUGAUGAA | 28740 | CUCAGCUGUGAGUU | 30782 | B cells |
| 4517 | ACUCACAGCUGAG | | UCAUCAUAUUU | | |
| hsa-miR- | GCUCAGGGAUGA | 28741 | UCUCAGCACAGUUA | 30783 | B cells |
| 4518 | UAACUGUGCUGA | | UCAUCCCUGAGC | | |
| | GA | | | | |
| hsa-miR- | CAGCAGUGCGCAG | 28742 | CAGCCCUGCGCACU | 30784 | B cells |
| 4519 | GGCUG | | GCUG | | |
| hsa-miR- | GCUAAGGAAGUC | 28743 | CUGAGCACAGGACU | 30785 | B cells |
| 4521 | CUGUGCUCAG | | UCCUUAGC | | |
| hsa-miR- | UGACUCUGCCUGU | 28744 | ACCGGCCUACAGGC | 30786 | B cells |
| 4522 | AGGCCGGU | | AGAGUCA | | |
| hsa-miR- | GACCGAGAGGGCC | 28745 | ACAGCCGAGGCCCU | 30787 | B cells |
| 4523 | UCGGCUGU | | CUCGGUC | | |
| hsa-miR- | GGGGGGAUGUGC | 28746 | AACCAGCAUGCACA | 30788 | B cells |
| 4525 | AUGCUGGUU | | UCCCCCC | | |
| hsa-miR- | UGGUCUGCAAAG | 28747 | ACAGUCAUCUCUUU | 30789 | B cells |
| 4527 | AGAUGACUGU | | GCAGACCA | | |
| hsa-miR- | UCAUUAUAUGUA | 28748 | GUCCAGAUCAUACA | 30790 | B cells |
| 4528 | UGAUCUGGAC | | UAUAAUGA | | |
| hsa-miR- | CCCAGCAGGACGG | 28749 | CGCUCCCGUCCUGC | 30791 | B cells |
| 4530 | GAGCG | | UGGG | | |
| hsa-miR- | AUGGAGAAGGCU | 28750 | UCAGAAGCCUUCUC | 30792 | B cells |
| 4531 | UCUGA | | CAU | | |
| hsa-miR- | CCCCGGGGAGCCC | 28751 | CGCCGGGCUCCCCG | 30793 | B cells |
| 4532 | GGCG | | GGG | | |
| hsa-miR- | UGGAAGGAGGUU | 28752 | AGCGUCCGGCAACC | 30794 | B cells |
| 4533 | GCCGGACGCU | | UCCUUCCA | | |
| hsa-miR- | GGAUGGAGGAGG | 28753 | AGACCCCUCCUCCA | 30795 | B cells |
| 4534 | GGUCU | | UCC | | |
| hsa-miR- | GUGGACCUGGCU | 28754 | GUCCCAGCCAGGUC | 30796 | B cells |
| 4535 | GGGAC | | CAC | | |
| hsa-miR- | UCGUGCAUAUAU | 28755 | AUGUGGUAGAUAUA | 30797 | B cells |
| 4536-3p | CUACCACAU | | UGCACGA | | |
| hsa-miR- | UGUGGUAGAUAU | 28756 | AUCGUGCAUAUAUC | 30798 | B cells |
| 4536-5p | AUGCACGAU | | UACCACA | | |
| hsa-miR- | UGAGCCGAGCUG | 28757 | CAGCUAAGCUCAGC | 30799 | B cells |
| 4537 | AGCUUAGCUG | | UCGGCUCA | | |
| hsa-miR- | GAGCUUGGAUGA | 28758 | UCAGCCCAGCUCAU | 30800 | B cells |
| 4538 | GCUGGGCUGA | | CCAAGCUC | | |
| hsa-miR- | GCUGAACUGGGC | 28759 | GCCCAGCUCAGCCC | 30801 | B cells |
| 4539 | UGAGCUGGGC | | AGUUCAGC | | |
| hsa-miR- | UUAGUCCUGCCUG | 28760 | UAAACCUACAGGCA | 30802 | B cells |
| 4540 | UAGGUUUA | | GGACUAA | | |
| hsa-miR- | UUGGGCUGGGCU | 28761 | CCCAACCCAGCCCA | 30803 | B-cells |
| 1587 | GGGUUGGG | | GCCCAA | | |
| hsa-miR- | UAGGAUGGGGGU | 28762 | CACCUCUCACCCCC | 30804 | B-cells |
| 2392 | GAGAGGUG | | AUCCUA | | |
| hsa-miR- | AAAAGUAAUUGU | 28763 | AGCAAAAUCCACAA | 30805 | B-cells |
| 548ab | GGAUUUUGCU | | UUACUUUU | | |
| hsa-miR- | CAAAAACCGGCAA | 28764 | CAAAAGUAAUUGCC | 30806 | B-cells |
| 548ac | UUACUUUUG | | GGUUUUUG | | |
| hsa-miR- | GAAAACGACAAU | 28765 | UGCAAAAGUCAUUG | 30807 | B-cells |
| 548ad | GACUUUUGCA | | UCGUUUUC | | |
| hsa-miR- | CAAAAACUGCAA | 28766 | UGAAAGUAAUUGCA | 30808 | B-cells |
| 548ae | UUACUUUCA | | GUUUUUG | | |
| hsa-miR- | AAAGGUAAUUGU | 28767 | GCAGAAACCACAAU | 30809 | B-cells |
| 548ag | GGUUUCUGC | | UACCUUU | | |
| hsa-miR- | CAAAAACUGCAG | 28768 | GCAAAAGUAACUGC | 30810 | B-cells |
| 548ah-3p | UUACUUUUGC | | AGUUUUUG | | |
| hsa-miR- | AAAAGUGAUUGC | 28769 | CAAACACUGCAAUC | 30811 | B-cells |
| 548ah-5p | AGUGUUUG | | ACUUUU | | |
| hsa-miR- | AAAGGUAAUUGC | 28770 | GGGAAAAACUGCAA | 30812 | B-cells |
| 548ai | AGUUUUUCCC | | UUACCUUU | | |
| hsa-miR- | UAAAAACUGCAA | 28771 | UAAAAGUAAUUGCA | 30813 | B-cells |
| 548aj-3p | UUACUUUUA | | GUUUUUA | | |
| hsa-miR- | UGCAAAAGUAAU | 28772 | CAAAAACUGCAAUU | 30814 | B-cells |
| 548aj-5p | UGCAGUUUUUG | | ACUUUUGCA | | |
| hsa-miR- | AAAAGUAACUGC | 28773 | UCAAAAACCGCAGU | 30815 | B-cells |
| 548ak | GGUUUUUGA | | UACUUUU | | |
| hsa-miR- | AACGGCAAUGAC | 28774 | UGGUACAAAAGUCA | 30816 | B-cells |
| 548al | UUUUGUACCA | | UUGCCGUU | | |
| hsa-miR- | CAAAAACUGCAG | 28775 | ACAAAAGUAACUGC | 30817 | B-cells |
| 548am-3p | UUACUUUUGU | | AGUUUUUG | | |
| hsa-miR- | AAAAGUAAUUGC | 28776 | GGCAAAAACCGCAA | 30818 | B-cells |
| 548am-5p | GGUUUUUGCC | | UUACUUUU | | |
| hsa-miR- | AAAAGGCAUUGU | 28777 | CAAAAACCACAAUG | 30819 | B-cells |
| 548an | GGUUUUUG | | CCUUUU | | |
| hsa-miR- | CGGCCCGGGCUGC | 28778 | AGGAACAGCAGCAG | 30820 | Blood |
| 1538 | UGCUGUUCCU | | CCCGGGCCG | | |
| hsa-miR- | AGAGGUAUAGGG | 28779 | UUCCCAUGCCCUAU | 30821 | Blood |
| 202-3p | CAUGGGAA | | ACCUCU | | |
| hsa-miR- | UUCCUAUGCAUA | 28780 | CAAAGAAGUAUAUG | 30822 | Blood |
| 202-5p | UACUUCUUUG | | CAUAGGAA | | |
| hsa-miR- | AUCAUAGAGGAA | 28781 | AACAUGGAUUUUCC | 30823 | Blood |
| 376b-3p | AAUCCAUGUU | | UCUAUGAU | | |
| hsa-miR- | CGUGGAUAUUCC | 28782 | AAACAUAGAAGGAA | 30824 | Blood |
| 376b-5p | UUCUAUGUUU | | UAUCCACG | | |
| hsa-miR- | UGAGUAUUACAU | 28783 | GAGAUUGGCCAUGU | 30825 | Blood |
| 496 | GGCCAAUCUC | | AAUACUCA | | |
| hsa-miR- | CUUCCGCCCCGCC | 28784 | CGACGCCCGGCGGG | 30826 | Blood |
| 718 | GGGCGUCG | | GCGGAAG | | |
| hsa-miR- | UAGCUUAUCAGA | 28785 | UCAACAUCAGUCUG | 30827 | Blood ( myeloid |
| 21-5p | CUGAUGUUGA | | AUAAGCUA | | cells), liver, |
| | | | | | endothelial cells |
| hsa-miR- | CACUAGAUUGUG | 28786 | UCCAGGAGCUCACA | 30828 | Blood (immune |
| 28-3p | AGCUCCUGGA | | AUCUAGUG | | cells) |
| hsa-miR- | AAGGAGCUCACA | 28787 | CUCAAUAGACUGUG | 30829 | Blood (immune |
| 28-5p | GUCUAUUGAG | | AGCUCCUU | | cells) |
| hsa-miR- | UACGUCAUCGUU | 28788 | UGACGAUGACAACG | 30830 | Blood |
| 598 | GUCAUCGUCA | | AUGACGUA | | (lymphocytes) |
| hsa-miR- | CACGCUCAUGCAC | 28789 | UGUGGGUGUGUGCA | 30831 | Blood (myeloid |
| 574-3p | ACACCCACA | | UGAGCGUG | | cells) |
| hsa-miR- | CAACACCAGUCGA | 28790 | ACAGCCCAUCGACU | 30832 | Blood (myeloid |
| 21-3p | UGGGCUGU | | GGUGUUG | | cells), glioblast, |
| | | | | | liver, vascular |
| | | | | | endothelial cells |
| hsa-miR- | UUCACCACCUUCU | 28791 | GCUGGGUGGAGAAG | 30833 | Blood (myeloid), |
| 197-3p | CCACCCAGC | | GUGGUGAA | | other tissues/cells |
| hsa-miR- | CGGGUAGAGAGG | 28792 | CCUCCCACUGCCCU | 30834 | Blood (myeloid), |
| 197-5p | GCAGUGGGAGG | | CUCUACCCG | | other tissues/cells |
| hsa-miR- | AAUCCUUGCUACC | 28793 | ACCCAGGUAGCAAG | 30835 | Blood (plasma) |
| 500b | UGGGU | | GAUU | | |
| hsa-miR- | CAAUUUAGUGUG | 28794 | AAAUAUCACACACA | 30836 | Blood and glia |
| 32-3p | UGUGAUAUUU | | CUAAAUUG | | |
| hsa-miR- | UAUUGCACAUUA | 28795 | UGCAACUUAGUAAU | 30837 | Blood and glia |
| 32-5p | CUAAGUUGCA | | GUGCAAUA | | |
| hsa-miR- | CCUAUUCUUGAU | 28796 | GAAACAAGUAAUCA | 30838 | Blood and other |
| 26a-2-3p | UACUUGUUUC | | AGAAUAGG | | tissues |
| hsa-miR- | UUCAAGUAAUCC | 28797 | AGCCUAUCCUGGAU | 30839 | Blood and other |
| 26a-5p | AGGAUAGGCU | | UACUUGAA | | tissues |
| hsa-miR- | CCAGUUACCGCUU | 28798 | GCGGUAGCGGAAGC | 30840 | Blood mononuclear |
| 935 | CCGCUACCGC | | GGUAACUGG | | cells |
| hsa-miR- | GAUUUCAGUGGA | 28799 | GAACUUCACUCCAC | 30841 | Blood(plasma) |
| 205-3p | GUGAAGUUC | | UGAAAUC | | |
| hsa-miR- | UCCUUCAUUCCAC | 28800 | CAGACUCCGGUGGA | 30842 | Blood(plasma) |
| 205-5p | CGGAGUCUG | | AUGAAGGA | | |
| hsa-miR- | AAAAUGGUGCCC | 28801 | UGUAGUCACUAGGG | 30843 | Blood(plasma), |
| 224-3p | UAGUGACUACA | | CACCAUUUU | | ovary |
| hsa-miR- | CAAGUCACUAGU | 28802 | AACGGAACCACUAG | 30844 | Blood(plasma) |
| 224-5p | GGUUCCGUU | | UGACUUG | | ovary |
| hsa-miR- | CCAGUAUUAACU | 28803 | UCAGCAGCACAGUU | 30845 | Blood, embryonic |
| 16-1-3p | GUGCUGCUGA | | AAUACUGG | | stem cells, |
| | | | | | hematopoietic |
| | | | | | tissues (spleen) |
| hsa-miR- | UCCCCCAGGUGUG | 28804 | AAAUCAGAAUCACA | 30846 | Blood, endothelial |
| 361-3p | AUUCUGAUUU | | CCUGGGGGA | | cells |
| hsa-miR- | AAAAGCUGGGUU | 28805 | UCGCCCUCUCAACC | 30847 | Blood, heart |
| 320a | GAGAGGGCGA | | CAGCUUUU | | (myocardial) |
| hsa-miR- | UGGACGGAGAAC | 28806 | ACCCUUAUCAGUUC | 30848 | Blood, tongue, |
| 184 | UGAUAAGGGU | | UCCGUCCA | | pancreas (islet) |
| hsa-miR- | UGGUGGGCCGCA | 28807 | GCACAUGUUCUGCG | 30849 | Bone marrow |
| 654-5p | GAACAUGUGC | | GCCCACCA | | |
| hsa-miR- | AGUGCCUGCUAU | 28808 | UGCCUGGCACAUAG | 30850 | Brain |
| 1271-3p | GUGCCAGGCA | | CAGGCACU | | |
| hsa-miR- | CUUGGCACCUAGC | 28809 | UGAGUGCUUGCUAG | 30851 | Brain |
| 1271-5p | AAGCACUCA | | GUGCCAAG | | |
| hsa-miR- | UUAUUGCUUAAG | 28810 | CUACGCGUAUUCUU | 30852 | Brain |
| 137 | AAUACGCGUAG | | AAGCAAUAA | | |
| hsa-miR- | UUGCAUAGUCAC | 28811 | GAUCACUUUUGUGA | 30853 | Brain |
| 153 | AAAAGUGAUC | | CUAUGCAA | | |
| hsa-miR- | GUGAAUUACCGA | 28812 | UUAUGGCCCUUCGG | 30854 | Brain |
| 183-3p | AGGGCCAUAA | | UAAUUCAC | | |
| hsa-miR- | UAUGGCACUGGU | 28813 | AGUGAAUUCUACCA | 30855 | Brain |
| 183-5p | AGAAUUCACU | | GUGCCAUA | | |
| hsa-miR- | UGAUAUGUUUGA | 28814 | ACCUAAUAUAUCAA | 30856 | Brain |
| 190a | UAUAUUAGGU | | ACAUAUCA | | |
| hsa-miR- | UGAUAUGUUUGA | 28815 | AACCCAAUAUCAAA | 30857 | Brain |
| 190b | UAUUGGGUU | | CAUAUCA | | |
| hsa-miR- | AGCAGGUGCGGG | 28816 | CGCCGCCCCGCACC | 30858 | Brain |
| 3665 | GCGGCG | | UGCU | | |
| hsa-miR- | CAGUGCAAGUGU | 28817 | UCGGCAUCUACACU | 30859 | Brain |
| 3666 | AGAUGCCGA | | UGCACUG | | |
| hsa-miR- | UAUGUAAUAUGG | 28818 | AAGAUGUGGACCAU | 30860 | Brain |
| 380-3p | UCCACAUCUU | | AUUACAUA | | |
| hsa-miR- | AAUAUAACACAG | 28819 | ACAGGCCAUCUGUG | 30861 | Brain |
| 410 | AUGGCCUGU | | UUAUAUU | | |
| hsa-miR- | AUCGGGAAUGUC | 28820 | GGGCGGACACGACA | 30862 | Brain |
| 425-3p | GUGUCCGCCC | | UUCCCGAU | | |
| hsa-miR- | AAUGACACGAUC | 28821 | UCAACGGGAGUGAU | 30863 | Brain |
| 425-5p | ACUCCCGUUGA | | CGUGUCAUU | | |
| hsa-miR- | UACUCAGGAGAG | 28822 | GUGAUUGCCACUCU | 30864 | Brain |
| 510 | UGGCAAUCAC | | CCUGAGUA | | |
| hsa-miR-7- | UGGAAGACUAGU | 28823 | ACAACAAAAUCACU | 30865 | Brain |
| 5p | GAUUUUGUUGU | | AGUCUUCCA | | |
| hsa-miR-9- | AUAAAGCUAGAU | 28824 | ACUUUCGGUUAUCU | 30866 | Brain |
| 3p | AACCGAAAGU | | AGCUUUAU | | |
| hsa-miR-9- | UCUUUGGUUAUC | 28825 | UCAUACAGCUAGAU | 30867 | Brain |
| 5p | UAGCUGUAUGA | | AACCAAAGA | | |
| hsa-miR- | ACAGGUGAGGUU | 28826 | GGCUCCCAAGAACC | 30868 | Brain and |
| 125a-3p | CUUGGGAGCC | | UCACCUGU | | hematopoietic cells |
| hsa-miR- | UCCCUGAGACCCU | 28827 | UCACAGGUUAAAGG | 30869 | Brain and |
| 125a-5p | UUAACCUGUGA | | GUCUCAGGGA | | hematopoietic cells |
| hsa-miR-7- | CAACAAAUCCCAG | 28828 | UUAGGUAGACUGGG | 30870 | Brain and pancreas |
| 2-3p | UCUACCUAA | | AUUUGUUG | | |
| hsa-miR- | CGUGUUCACAGCG | 28829 | AUCAAGGUCCGCUG | 30871 | Brain and plasma |
| 124-5p | GACCUUGAU | | UGAACACG | | (circulating) |
| hsa-miR- | UAAGGCACGCGG | 28830 | GGCAUUCACCGCGU | 30872 | Brain and plasma |
| 124-3p | UGAAUGCC | | GCCUUA | | (exosomal) |
| hsa-miR- | AACACACCUGGUU | 28831 | AAAGAGGUUAACCA | 30873 | Brain and platelet |
| 329 | AACCUCUUU | | GGUGUGUU | | |
| hsa-miR- | UAACAGUCUACA | 28832 | CGACCAUGGCUGUA | 30874 | Brain(neuron), |
| 132-3p | GCCAUGGUCG | | GACUGUUA | | immune cells |
| hsa-miR- | ACCGUGGCUUUCG | 28833 | AGUAACAAUCGAAA | 30875 | Brain(neuron), |
| 132-5p | AUUGUUACU | | GCCACGGU | | immune cells |
| hsa-miR- | UAACAGUCUCCAG | 28834 | GGCCGUGACUGGAG | 30876 | Brain(ncuron), |
| 212-3p | UCACGGCC | | ACUGUUA | | spleen |
| hsa-miR- | ACCUUGGCUCUAG | 28835 | AGUAAGCAGUCUAG | 30877 | Brain(neuron), |
| 212-5p | ACUGCUUACU | | AGCCAAGGU | | spleen |
| hsa-miR- | UCUCACACAGAAA | 28836 | ACGGGUGCGAUUUC | 30878 | Brain, circulating |
| 342-3p | UCGCACCCGU | | UGUGUGAGA | | plasma |
| hsa-miR- | UGGUUGACCAUA | 28837 | GCGCAUGUUCUAUG | 30879 | Brain, embryonic |
| 380-5p | GAACAUGCGC | | GUCAACCA | | stem cells |
| hsa-miR- | CAGUAACAAAGA | 28838 | ACAAGGAUGAAUCU | 30880 | Brain, epithelial |
| 802 | UUCAUCCUUGU | | UUGUUACUG | | cells, hepatocytes |
| hsa-miR- | AGAGUUGAGUCU | 28839 | CGGGACGUCCAGAC | 30881 | Brain, |
| 219-1-3p | GGACGUCCCG | | UCAACUCU | | oligodendrocytes |
| hsa-miR- | AGAAUUGUGGCU | 28840 | ACAGAUGUCCAGCC | 30882 | Brain, |
| 219-2-3p | GGACAUCUGU | | ACAAUUCU | | oligodendrocytes |
| hsa-miR- | UGAUUGUCCAAA | 28841 | AGAAUUGCGUUUGG | 30883 | Brain, |
| 219-5p | CGCAAUUCU | | ACAAUCA | | oligodendrocytes |
| hsa-miR- | UAUAGGGAUUGG | 28842 | CGCCACGGCUCCAA | 30884 | Brain, other tissues |
| 135a-3p | AGCCGUGGCG | | UCCCUAUA | | |
| hsa-miR- | UAUGGCUUUUUA | 28843 | UCACAUAGGAAUAA | 30885 | Brain, other tissues |
| 135a-5p | UUCCUAUGUGA | | AAAGCCAUA | | |
| hsa-miR- | AUGUAGGGCUAA | 28844 | CCCAUGGCUUUUAG | 30886 | Brain, placenta, |
| 135b-3p | AAGCCAUGGG | | CCCUACAU | | other tissues |
| hsa-miR- | UAUGGCUUUUCA | 28845 | UCACAUAGGAAUGA | 30887 | Brain, placenta, |
| 135b-5p | UUCCUAUGUGA | | AAAGCCAUA | | other tissues |
| hsa-miR- | AACCAUCGACCGU | 28846 | GUCCACUCAACGGU | 30888 | Brain, stem |
| 181c-3p | UGAGUGGAC | | CGAUGGUU | | cells/progenitor |
| hsa-miR- | AACAUUCAACCUG | 28847 | ACUCACCGACAGGU | 30889 | Brain, stem |
| 181c-5p | UCGGUGAGU | | UGAAUGUU | | cells/progenitor |
| hsa-miR- | CUUCCAGACGCUC | 28848 | CGACGUGGGGCGGA | 30890 | Breast tumor |
| 3180-5p | CGCCCCACGUCG | | GCGUCUGGAAG | | |
| hsa-miR- | AACAACAAAAUC | 28849 | UGGAAGACUAGUGA | 30891 | Breast tumor |
| 3529-3p | ACUAGUCUUCCA | | UUUUGUUGUU | | |
| hsa-miR- | AGGUAGACUGGG | 28850 | AACAACAAAUCCCA | 30892 | Breast tumor |
| 3529-5p | AUUUGUUGUU | | GUCUACCU | | |
| hsa-miR- | AAACACCAUUGUC | 28851 | GUGGAGUGUGACAA | 30893 | Breast tumor |
| 3591-3p | ACACUCCAC | | UGGUGUUU | | |
| hsa-miR- | UUUAGUGUGAUA | 28852 | UCAAACGCCAUUAU | 30894 | Breast tumor |
| 3591-5p | AUGGCGUUUGA | | CACACUAAA | | |
| hsa-miR- | UAUGGAAAGACU | 28853 | AGAGUGGCAAAGUC | 30895 | Breast tumor |
| 3688-3p | UUGCCACUCU | | UUUCCAUA | | |
| hsa-miR- | AGUGGCAAAGUC | 28854 | AUAUGGAAAGACUU | 30896 | Breast tumor |
| 3688-5p | UUUCCAUAU | | UGCCACU | | |
| hsa-miR- | CAGCCCGGAUCCC | 28855 | AAGUGGGCUGGGAU | 30897 | Breast tumor |
| 3940-3p | AGCCCACUU | | CCGGGCUG | | |
| hsa-miR- | GUGGGUUGGGGC | 28856 | CAGAGCCCGCCCCA | 30898 | Breast tumor |
| 3940-5p | GGGCUCUG | | ACCCAC | | |
| hsa-miR- | UUCGGGCUGGCCU | 28857 | CCGGAGCAGCAGGC | 30899 | Breast tumor |
| 3944-3p | GCUGCUCCGG | | CAGCCCGAA | | |
| hsa-miR- | UGUGCAGCAGGCC | 28858 | UCUCGGUUGGCCUG | 30900 | Breast tumor |
| 3944-5p | AACCGAGA | | CUGCACA | | |
| hsa-miR- | CAGGGCAGGAAG | 28859 | UUGUCCACUUCUUC | 30901 | Breast tumor |
| 4436b-3p | AAGUGGACAA | | CUGCCCUG | | |
| hsa-miR- | GUCCACUUCUGCC | 28860 | GGCAGGGCAGGCAG | 30902 | Breast tumor |
| 4436b-5p | UGCCCUGCC | | AAGUGGAC | | |
| hsa-miR- | UUUGGACAGAAA | 28861 | ACCUGCGUGUUUUC | 30903 | Breast tumor |
| 4520b-3p | ACACGCAGGU | | UGUCCAAA | | |
| hsa-miR- | CCUGCGUGUUUUC | 28862 | UUGGACAGAAAACA | 30904 | Breast tumor |
| 4520b-5p | UGUCCAA | | CGCAGG | | |
| hsa-miR- | UGCCGCCCUCUCG | 28863 | CUAGAGCAGCGAGA | 30905 | Breast tumor |
| 4632-3p | CUGCUCUAG | | GGGCGGCA | | |
| hsa-miR- | GAGGGCAGCGUG | 28864 | UCCGCCACACCCAC | 30906 | Breast tumor |
| 4632-5p | GGUGUGGCGGA | | GCUGCCCUC | | |
| hsa-miR- | AGGAGCUAGCCA | 28865 | UGCAUAUGCCUGGC | 30907 | Breast tumor |
| 4633-3p | GGCAUAUGCA | | UAGCUCCU | | |
| hsa-miR- | AUAUGCCUGGCU | 28866 | GAGGAGCUAGCCAG | 30908 | Breast tumor |
| 4633-5p | AGCUCCUC | | GCAUAU | | |
| hsa-miR- | CGGCGCGACCGGC | 28867 | CCCCGGGCCGGUCG | 30909 | Breast tumor |
| 4634 | CCGGGG | | CGCCG | | |
| hsa-miR- | UCUUGAAGUCAG | 28868 | UUGCGGGUUCUGAC | 30910 | Breast tumor |
| 4635 | AACCCGCAA | | UUCAAGA | | |
| hsa-miR- | AACUCGUGUUCA | 28869 | CUAAAGGCUUUGAA | 30911 | Breast tumor |
| 4636 | AAGCCUUUAG | | CACGAGUU | | |
| hsa-miR- | CCUGGACACCGCU | 28870 | CGGCCGGCUGAGCG | 30912 | Breast tumor |
| 4638-3p | CAGCCGGCCG | | GUGUCCAGG | | |
| hsa-miR- | ACUCGGCUGCGGU | 28871 | ACUUGUCCACCGCA | 30913 | Breast tumor |
| 4638-5p | GGACAAGU | | GCCGAGU | | |
| hsa-miR- | UCACUCUCACCUU | 28872 | GCAAAGCAAGGUGA | 30914 | Breast tumor |
| 4639-3p | GCUUUGC | | GAGUGA | | |
| hsa-miR- | UUGCUAAGUAGG | 28873 | UCAAUCUCAGCCUA | 30915 | Breast tumor |
| 4639-5p | CUGAGAUUGA | | CUUAGCAA | | |
| hsa-miR- | CACCCCCUGUUUC | 28874 | GUGGGCCAGGAAAC | 30916 | Breast tumor |
| 4640-3p | CUGGCCCAC | | AGGGGGUG | | |
| hsa-miR- | UGGGCCAGGGAG | 28875 | CCCACCAGCUGCUC | 30917 | Breast tumor |
| 4640-5p | CAGCUGGUGGG | | CCUGGCCCA | | |
| hsa-miR- | UGCCCAUGCCAUA | 28876 | UGAGGCAAAAGUAU | 30918 | Breast tumor |
| 4641 | CUUUUGCCUCA | | GGCAUGGGCA | | |
| hsa-miR- | AUGGCAUCGUCCC | 28877 | AGCCACCAGGGGAC | 30919 | Breast tumor |
| 4642 | CUGGUGGCU | | GAUGCCAU | | |
| hsa-miR- | GACACAUGACCAU | 28878 | UUAGCAUUUAUGGU | 30920 | Breast tumor |
| 4643 | AAAUGCUAA | | CAUGUGUC | | |
| hsa-miR- | UGGAGAGAGAAA | 28879 | CUUCUGUCUCUUUU | 30921 | Breast tumor |
| 4644 | AGAGACAGAAG | | CUCUCUCCA | | |
| hsa-miR- | AGACAGUAGUUC | 28880 | AACCAGGCAAGAAC | 30922 | Breast tumor |
| 4645-3p | UUGCCUGGUU | | UACUGUCU | | |
| hsa-miR- | ACCAGGCAAGAA | 28881 | ACAAUAUUUCUUGC | 30923 | Breast tumor |
| 4645-5p | AUAUUGU | | CUGGU | | |
| hsa-miR- | AUUGUCCCUCUCC | 28882 | CUGGGAAGGGAGAG | 30924 | Breast tumor |
| 4646-3p | CUUCCCAG | | GGACAAU | | |
| hsa-miR- | ACUGGGAAGAGG | 28883 | UCCCUCAGCUCCUC | 30925 | Breast tumor |
| 4646-5p | AGCUGAGGGA | | UUCCCAGU | | |
| hsa-miR- | GAAGAUGGUGCU | 28884 | UUCCUCAGCACAGC | 30926 | Breast tumor |
| 4647 | GUGCUGAGGAA | | ACCAUCUUC | | |
| hsa-miR- | UGUGGGACUGCA | 28885 | CUCCCAUUUGCAGU | 30927 | Breast tumor |
| 4648 | AAUGGGAG | | CCCACA | | |
| hsa-miR- | UCUGAGGCCUGCC | 28886 | UGGGGAGAGGCAGG | 30928 | Breast tumor |
| 4649-3p | UCUCCCCA | | CCUCAGA | | |
| hsa-miR- | UGGGCGAGGGGU | 28887 | CUCUGAGAGCCCAC | 30929 | Breast tumor |
| 4649-5p | GGGCUCUCAGAG | | CCCUCGCCCA | | |
| hsa-miR- | AGGUAGAAUGAG | 28888 | AUGUCAGGCCUCAU | 30930 | Breast tumor |
| 4650-3p | GCCUGACAU | | UCUACCU | | |
| hsa-miR- | UCAGGCCUCUUUC | 28889 | AAGGUAGAAAGAGG | 30931 | Breast tumor |
| 4650-5p | UACCUU | | CCUGA | | |
| hsa-miR- | CGGGGUGGGUGA | 28890 | GCCCGACCUCACCC | 30932 | Breast tumor |
| 4651 | GGUCGGGC | | ACCCCG | | |
| hsa-miR- | GUUCUGUUAACCC | 28891 | UGAGGGGAUGGGUU | 30933 | Breast tumor |
| 4652-3p | AUCCCCUCA | | AACAGAAC | | |
| hsa-miR- | AGGGGACUGGUU | 28892 | UAGUUCUAUUAACC | 30934 | Breast tumor |
| 4652-5p | AAUAGAACUA | | AGUCCCCU | | |
| hsa-miR- | UGGAGUUAAGGG | 28893 | UCUCCAAGCAACCC | 30935 | Breast tumor |
| 4653-3p | UUGCUUGGAGA | | UUAACUCCA | | |
| hsa-miR- | UCUCUGAGCAAG | 28894 | GGUGUUAAGCCUUG | 30936 | Breast tumor |
| 4653-5p | GCUUAACACC | | CUCAGAGA | | |
| hsa-miR- | UGUGGGAUCUGG | 28895 | CCAGAUGCCUCCAG | 30937 | Breast tumor |
| 4654 | AGGCAUCUGG | | AUCCCACA | | |
| hsa-miR- | ACCCUCGUCAGGU | 28896 | CCCCGGGGACCUGA | 30938 | Breast tumor |
| 4655-3p | CCCCGGGG | | CGAGGGU | | |
| hsa-miR- | CACCGGGGAUGGC | 28897 | CGACCCUCUGCCAU | 30939 | Breast tumor |
| 4655-5p | AGAGGGUCG | | CCCCGGUG | | |
| hsa-miR- | UGGGCUGAGGGC | 28898 | ACAGGCCUCCUGCC | 30940 | Breast tumor |
| 4656 | AGGAGGCCUGU | | CUCAGCCCA | | |
| hsa-miR- | AAUGUGGAAGUG | 28899 | AUGCCUCAGACCAC | 30941 | Breast tumor |
| 4657 | GUCUGAGGCAU | | UUCCACAUU | | |
| hsa-miR- | GUGAGUGUGGAU | 28900 | AUUCCUCCAGGAUC | 30942 | Breast tumor |
| 4658 | CCUGGAGGAAU | | CACACUCAC | | |
| hsa-miR- | UUUCUUCUUAGA | 28901 | CGUUGCCAUGUCUA | 30943 | Breast tumor |
| 4659a-3p | CAUGGCAACG | | AGAAGAAA | | |
| hsa-miR- | CUGCCAUGUCUAA | 28902 | GUUUUCUUCUUAGA | 30944 | Breast tumor |
| 4659a-5p | GAAGAAAAC | | CAUGGCAG | | |
| hsa-miR- | UUUCUUCUUAGA | 28903 | AGCUGCCAUGUCUA | 30945 | Breast tumor |
| 4659b-3p | CAUGGCAGCU | | AGAAGAAA | | |
| hsa-miR- | UUGCCAUGUCUA | 28904 | UUCUUCUUAGACAU | 30946 | Breast tumor |
| 4659b-5p | AGAAGAA | | GGCAA | | |
| hsa-miR- | UGCAGCUCUGGU | 28905 | CUCCAUUUUCCACC | 30947 | Breast tumor |
| 4660 | GGAAAAUGGAG | | AGAGCUGCA | | |
| hsa-miR- | CAGGAUCCACAGA | 28906 | UGGACUAGCUCUGU | 30948 | Breast tumor |
| 4661-3p | GCUAGUCCA | | GGAUCCUG | | |
| hsa-miR- | AACUAGCUCUGU | 28907 | GUCAGGAUCCACAG | 30949 | Breast tumor |
| 4661-5p | GGAUCCUGAC | | AGCUAGUU | | |
| hsa-miR- | AAAGAUGGACAA | 28908 | AUUUAGCCAAUUGU | 30950 | Breast tumor |
| 4662b | UUGGCUAAAU | | CCAUCUUU | | |
| hsa-miR- | AGCUGAGCUCCAU | 28909 | ACUGCACGUCCAUG | 30951 | Breast tumor |
| 4663 | GGACGUGCAGU | | GAGCUCAGCU | | |
| hsa-miR- | CUUCCGGUCUGUG | 28910 | GACGGGGCUCACAG | 30952 | Breast tumor |
| 4664-3p | AGCCCCGUC | | ACCGGAAG | | |
| hsa-miR- | UGGGGUGCCCACU | 28911 | AACUUGCGGAGUGG | 30953 | Breast tumor |
| 4664-5p | CCGCAAGUU | | GCACCCCA | | |
| hsa-miR- | CUCGGCCGCGGCG | 28912 | GGCGGGGGCUACGC | 30954 | Breast tumor |
| 4665-3p | CGUAGCCCCCGCC | | GCCGCGGCCGAG | | |
| hsa-miR- | CUGGGGGACGCG | 28913 | GCUCGCGCUCACGC | 30955 | Breast tumor |
| 4665-5p | UGAGCGCGAGC | | GUCCCCCAG | | |
| hsa-miR- | CAUACAAUCUGAC | 28914 | AAAUACAUGUCAGA | 30956 | Breast tumor |
| 4666a-3p | AUGUAUUU | | UUGUAUG | | |
| hsa-miR- | AUACAUGUCAGA | 28915 | GGCAUACAAUCUGA | 30957 | Breast tumor |
| 4666a-5p | UUGUAUGCC | | CAUGUAU | | |
| hsa-miR- | UCCCUCCUUCUGU | 28916 | CUGUGGGGACAGAA | 30958 | Breast tumor |
| 4667-3p | CCCCACAG | | GGAGGGA | | |
| hsa-miR- | ACUGGGGAGCAG | 28917 | GGUUCUCCUUCUGC | 30959 | Breast tumor |
| 4667-5p | AAGGAGAACC | | UCCCCAGU | | |
| hsa-miR- | GAAAAUCCUUUU | 28918 | CUGGAAAAACAAAA | 30960 | Breast tumor |
| 4668-3p | UGUUUUUCCAG | | AGGAUUUUC | | |
| hsa-miR- | AGGGAAAAAAAA | 28919 | GACAAAUCCUUUUU | 30961 | Breast tumor |
| 4668-5p | AAGGAUUUGUC | | UUUUUCCCU | | |
| hsa-miR- | UGUGUCCGGGAA | 28920 | CCUCCUCCACUUCC | 30962 | Breast tumor |
| 4669 | GUGGAGGAGG | | CGGACACA | | |
| hsa-miR- | UGAAGUUACAUC | 28921 | AAGCGACCAUGAUG | 30963 | Breast tumor |
| 4670-3p | AUGGUCGCUU | | UAACUUCA | | |
| hsa-miR- | AAGCGACCAUGA | 28922 | UGAAGUUACAUCAU | 30964 | Breast tumor |
| 4670-5p | UGUAACUUCA | | GGUCGCUU | | |
| hsa-miR- | UUAGUGCAUAGU | 28923 | AGACCAAAGACUAU | 30965 | Breast tumor |
| 4671-3p | CUUUGGUCU | | GCACUAA | | |
| hsa-miR- | ACCGAAGACUGU | 28924 | AGAUUAGCGCACAG | 30966 | Breast tumor |
| 4671-5p | GCGCUAAUCU | | UCUUCGGU | | |
| hsa-miR- | UUACACAGCUGG | 28925 | UGCCUCUGUCCAGC | 30967 | Breast tumor |
| 4672 | ACAGAGGCA | | UGUGUAA | | |
| hsa-miR- | UCCAGGCAGGAGC | 28926 | UCCAGUCCGGCUCC | 30968 | Breast tumor |
| 4673 | CGGACUGGA | | UGCCUGGA | | |
| hsa-miR- | CUGGGCUCGGGAC | 28927 | AGCCGCGCGUCCCG | 30969 | Breast tumor |
| 4674 | GCGCGGCU | | AGCCCAG | | |
| hsa-miR- | GGGGCUGUGAUU | 28928 | CCUGCUGGUCAAUC | 30970 | Breast tumor |
| 4675 | GACCAGCAGG | | ACAGCCCC | | |
| hsa-miR- | CACUGUUUCACCA | 28929 | AAGAGCCAGUGGUG | 30971 | Breast tumor |
| 4676-3p | CUGGCUCUU | | AAACAGUG | | |
| hsa-miR- | GAGCCAGUGGUG | 28930 | UCACUGUCUCACCA | 30972 | Breast tumor |
| 4676-5p | AGACAGUGA | | CUGGCUC | | |
| hsa-miR- | AAGGUAUUGUUC | 28931 | UCAUAAGUCUGAAC | 30973 | Breast tumor |
| 4678 | AGACUUAUGA | | AAUACCUU | | |
| hsa-miR- | UCUGUGAUAGAG | 28932 | AGCAAAGAAUCUCU | 30974 | Breast tumor |
| 4679 | AUUCUUUGCU | | AUCACAGA | | |
| hsa-miR- | UCUGAAUUGUAA | 28933 | UAACAACUCUUACA | 30975 | Breast tumor |
| 4680-3p | GAGUUGUUA | | AUUCAGA | | |
| hsa-miR- | AGAACUCUUGCA | 28934 | ACAUCUAAGACUGC | 30976 | Breast tumor |
| 4680-5p | GUCUUAGAUGU | | AAGAGUUCU | | |
| hsa-miR- | AACGGGAAUGCA | 28935 | AGAUACAGCCUGCA | 30977 | Breast tumor |
| 4681 | GGCUGUAUCU | | UUCCCGUU | | |
| hsa-miR- | UCUGAGUUCCUG | 28936 | AGACCAGGCUCCAG | 30978 | Breast tumor |
| 4682 | GAGCCUGGUCU | | GAACUCAGA | | |
| hsa-miR- | UGGAGAUCCAGU | 28937 | AUCGGGCGAGCACU | 30979 | Breast tumor |
| 4683 | GCUCGCCCGAU | | GGAUCUCCA | | |
| hsa-miR- | UGUUGCAAGUCG | 28938 | ACGUCUCCACCGAC | 30980 | Breast tumor |
| 4684-3p | GUGGAGACGU | | UUGCAACA | | |
| hsa-miR- | CUCUCUACUGACU | 28939 | UAUGUUGCAAGUCA | 30981 | Breast tumor |
| 4684-5p | UGCAACAUA | | GUAGAGAG | | |
| hsa-miR- | UCUCCCUUCCUGC | 28940 | CUAGCCAGGGCAGG | 30982 | Breast tumor |
| 4685-3p | CCUGGCUAG | | AAGGGAGA | | |
| hsa-miR- | CCCAGGGCUUGGA | 28941 | AACCUUGCCCCACU | 30983 | Breast tumor |
| 4685-5p | GUGGGGCAAGGU | | CCAAGCCCUGGG | | |
| | U | | | | |
| hsa-miR- | UAUCUGCUGGGC | 28942 | AACACCAGAAAGCC | 30984 | Breast tumor |
| 4686 | UUUCUGGUGUU | | CAGCAGAUA | | |
| hsa-miR- | UGGCUGUUGGAG | 28943 | GCCUGCCCCCUCCA | 30985 | Breast tumor |
| 4687-3p | GGGGCAGGC | | ACAGCCA | | |
| hsa-miR- | CAGCCCUCCUCCC | 28944 | UUUGGGUGCGGGAG | 30986 | Breast tumor |
| 4687-5p | GCACCCAAA | | GAGGGCUG | | |
| hsa-miR- | UAGGGGCAGCAG | 28945 | CCCAGGUCCUCUGC | 30987 | Breast tumor |
| 4688 | AGGACCUGGG | | UGCCCCUA | | |
| hsa-miR- | UUGAGGAGACAU | 28946 | GGCCCCCACCAUGU | 30988 | Breast tumor |
| 4689 | GGUGGGGGCC | | CUCCUCAA | | |
| hsa-miR- | GCAGCCCAGCUGA | 28947 | CAGAGGCCUCAGCU | 30989 | Breast tumor |
| 4690-3p | GGCCUCUG | | GGGCUGC | | |
| hsa-miR- | GAGCAGGCGAGG | 28948 | UUCAGCCCAGOCUC | 30990 | Breast tumor |
| 4690-5p | CUGGGCUGAA | | GCCUGCUC | | |
| hsa-miR- | CCAGCCACGGACU | 28949 | AUGCACUCUCAGUC | 30991 | Breast tumor |
| 4691-3p | GAGAGUGCAU | | CGUGGCUGG | | |
| hsa-miR- | GUCCUCCAGGCCA | 28950 | CCGCAGCUCAUGGC | 30992 | Breast tumor |
| 4691-5p | UGAGCUGCGG | | CUGGAGGAC | | |
| hsa-miR- | UCAGGCAGUGUG | 28951 | AUCUGAUACCCACA | 30993 | Breast tumor |
| 4692 | GGUAUCAGAU | | CUGCCUGA | | |
| hsa-miR- | UGAGAGUGGAAU | 28952 | AAAUACUGUGAAUU | 30994 | Breast tumor |
| 4693-3p | UCACAGUAUUU | | CCACUCUCA | | |
| hsa-miR- | AUACUGUGAAUU | 28953 | UGUGACAGUGAAAU | 30995 | Breast tumor |
| 4693-5p | UCACUGUCACA | | UCACAGUAU | | |
| hsa-miR- | CAAAUGGACAGG | 28954 | AGGUGUUAUCCUGU | 30996 | Breast tumor |
| 4694-3p | AUAACACCU | | CCAUUUG | | |
| hsa-miR- | AGGUGUUAUCCU | 28955 | GCAAAUGGAUAGGA | 30997 | Breast tumor |
| 4694-5p | AUCCAUUUGC | | UAACACCU | | |
| hsa-miR- | UGAUCUCACCGCU | 28956 | GAAGGAGGCAGCGG | 30998 | Breast tumor |
| 4695-3p | GCCUCCUUC | | UGAGAUCA | | |
| hsa-miR- | CAGGAGGCAGUG | 28957 | CCUGCUCGCCCACU | 30999 | Breast tumor |
| 4695-5p | GGCGAGCAGG | | GCCUCCUG | | |
| hsa-miR- | UGCAAGACGGAU | 28958 | AGAUGACAGUAUCC | 31000 | Breast tumor |
| 4696 | ACUGUCAUCU | | GUCUUGCA | | |
| hsa-miR- | UGUCAGUGACUCC | 28959 | ACCAAGGGGCAGGA | 31001 | Breast tumor |
| 4697-3p | UGCCCCUUGGU | | GUCACUGACA | | |
| hsa-miR- | AGGGGGCGCAGU | 28960 | CACGUCAGUGACUG | 31002 | Breast tumor |
| 4697-5p | CACUGACGUG | | CGCCCCCU | | |
| hsa-miR- | UCAAAAUGUAGA | 28961 | UGGGGUCUUCCUCU | 31003 | Breast tumor |
| 4698 | GGAAGACCCCA | | ACAUUUUGA | | |
| hsa-miR- | AAUUUACUCUGC | 28962 | GGAGAAGAUUGCAG | 31004 | Breast tumor |
| 4699-3p | AAUCUUCUCC | | AGUAAAUU | | |
| hsa-miR- | AGAAGAUUGCAG | 28963 | GGAACUUACUCUGC | 31005 | Breast tumor |
| 4699-5p | AGUAAGUUCC | | AAUCUUCU | | |
| hsa-miR- | CACAGGACUGACU | 28964 | CACUGGGGUGAGGA | 31006 | Breast tumor |
| 4700-3p | CCUCACCCCAGUG | | GUCAGUCCUGUG | | |
| hsa-miR- | UCUGGGGAUGAG | 28965 | ACACACUGUCCUCA | 31007 | Breast tumor |
| 4700-5p | GACAGUGUGU | | UCCCCAGA | | |
| hsa-miR- | AUGGGUGAUGGG | 28966 | ACACCACACCCAUC | 31008 | Breast tumor |
| 4701-3p | UGUGGUGU | | ACCCAU | | |
| hsa-miR- | UUGGCCACCACAC | 28967 | AAGGGGUAGGUGUG | 31009 | Breast tumor |
| 4701-5p | CUACCCCUU | | GUGGCCAA | | |
| hsa-miR- | UGUAGUUGUAUU | 28968 | GUGGCAAUACAAUA | 31010 | Breast tumor |
| 4703-3p | GUAUUGCCAC | | CAACUACA | | |
| hsa-miR- | UAGCAAUACAGU | 28969 | ACUAUAUUUGUACU | 31011 | Breast tumor |
| 4703-5p | ACAAAUAUAGU | | GUAUUGCUA | | |
| hsa-miR- | UCAGUCACAUAUC | 28970 | UAGACACUAGAUAU | 31012 | Breast tumor |
| 4704-3p | UAGUGUCUA | | GUGACUGA | | |
| hsa-miR- | GACACUAGGCAU | 28971 | AAUCACUCACAUGC | 31013 | Breast tumor |
| 4704-5p | GUGAGUGAUU | | CUAGUGUC | | |
| hsa-miR- | UCAAUCACUUGG | 28972 | ACAGCAAUUACCAA | 31014 | Breast tumor |
| 4705 | UAAUUGCUGU | | GUGAUUGA | | |
| hsa-miR- | AGCGGGGAGGAA | 28973 | AAGCAGCGCCCACU | 31015 | Breast tumor |
| 4706 | GUGGGCGCUGCU | | UCCUCCCCGCU | | |
| | U | | | | |
| hsa-miR- | AGCCCGCCCCAGC | 28974 | AGAACCUCGGCUGG | 31016 | Breast tumor |
| 4707-3p | CGAGGUUCU | | GGCGGGCU | | |
| hsa-miR- | GCCCCGGCGCGGG | 28975 | CCAGAACCCGCCCG | 31017 | Breast tumor |
| 4707-5p | CGGGUUCUGG | | CGCCGGGGC | | |
| hsa-miR- | AGCAAGGCGGCA | 28976 | AUCAGAGAGAUGCC | 31018 | Breast tumor |
| 4708-3p | UCUCUCUGAU | | GCCUUGCU | | |
| hsa-miR- | AGAGAUGCOGCCU | 28977 | AAGGAGCAAGGCGG | 31019 | Breast tumor |
| 4708-5p | UGCUCCUU | | CAUCUCU | | |
| hsa-miR- | UUGAAGAGGAGG | 28978 | GCUACAGAGCACCU | 31020 | Breast tumor |
| 4709-3p | UGCUCUGUAGC | | CCUCUUCAA | | |
| hsa-miR- | ACAACAGUGACU | 28979 | UUGGAGAGCAAGUC | 31021 | Breast tumor |
| 4709-5p | UGCUCUCCAA | | ACUGUUGU | | |
| hsa-miR- | GGGUGAGGGCAG | 28980 | AACCACCUGCCCUC | 31022 | Breast tumor |
| 4710 | GUGGUU | | ACCC | | |
| hsa-miR- | CGUGUCUUCUGGC | 28981 | AUCAAGCCAGAAGA | 31023 | Breast tumor |
| 4711-3p | UUGAU | | CACG | | |
| hsa-miR- | UGCAUCAGGCCAG | 28982 | CUCAUGUCUUCUGG | 31024 | Breast tumor |
| 4711-5p | AAGACAUGAG | | CCUGAUGCA | | |
| hsa-miR- | AAUGAGAGACCU | 28983 | AUACAGUACAGGUC | 31025 | Breast tumor |
| 4712-3p | GUACUGUAU | | UCUCAUU | | |
| hsa-miR- | UCCAGUACAGGUC | 28984 | GAAAUGAGAGACCU | 31026 | Breast tumor |
| 4712-5p | UCUCAUUUC | | GUACUGGA | | |
| hsa-miR- | UGGGAUCCAGAC | 28985 | UUCUCCCACUGUCU | 31027 | Breast tumor |
| 4713-3p | AGUGGGAGAA | | GGAUCCCA | | |
| hsa-miR- | UUCUCCCACUACC | 28986 | UGGGAGCCUGGUAG | 31028 | Breast tumor |
| 4713-5p | AGGCUCCCA | | UGGGAGAA | | |
| hsa-miR- | CCAACCUAGGUGG | 28987 | CAACUCUGACCACC | 31029 | Breast tumor |
| 4714-3p | UCAGAGUUG | | UAGGUUGG | | |
| hsa-miR- | AACUCUGACCCCU | 28988 | AUCAACCUAAGGGG | 31030 | Breast tumor |
| 4714-5p | UAGGUUGAU | | UCAGAGUU | | |
| hsa-miR- | GUGCCACCUUAAC | 28989 | AUUGGCUGCAGUUA | 31031 | Breast tumor |
| 4715-3p | UGCAGCCAAU | | AGGUGGCAC | | |
| hsa-miR- | AAGUUGGCUGCA | 28990 | CCACCUUAACUGCA | 31032 | Breast tumor |
| 4715-5p | GUUAAGGUGG | | GCCAACUU | | |
| hsa-miR- | AAGGGGGAAGGA | 28991 | UCUCCAUGUUUCCU | 31033 | Breast tumor |
| 4716-3p | AACAUGGAGA | | UCCCCCUU | | |
| hsa-miR- | UCCAUGUUUCCUU | 28992 | AGAAGGGGGAAGGA | 31034 | Breast tumor |
| 4716-5p | CCCCCUUCU | | AACAUGGA | | |
| hsa-miR- | ACACAUGGGUGG | 28993 | AGGCCACAGCCACC | 31035 | Breast tumor |
| 4717-3p | CUGUGGCCU | | CAUGUGU | | |
| hsa-miR- | UAGGCCACAGCCA | 28994 | ACACAUGGGUGGCU | 31036 | Breast tumor |
| 4717-5p | CCCAUGUGU | | GUGGCCUA | | |
| hsa-miR- | AGCUGUACCUGA | 28995 | UGCUUGGUUUCAGG | 31037 | Breast tumor |
| 4718 | AACCAAGCA | | UACAGCU | | |
| hsa-miR- | UCACAAAUCUAU | 28996 | CCUGCAUAUUAUAG | 31038 | Breast tumor |
| 4719 | AAUAUGCAGG | | AUUUGUGA | | |
| hsa-miR- | UGCUUAAGUUGU | 28997 | AUACUUGGUACAAC | 31039 | Breast tumor |
| 4720-3p | ACCAAGUAU | | UUAAGCA | | |
| hsa-miR- | CCUGGCAUAUUU | 28998 | AAGUUAUACCAAAU | 31040 | Breast tumor |
| 4720-5p | GGUAUAACUU | | AUGCCAGG | | |
| hsa-miR- | UGAGGGCUCCAG | 28999 | CCACCGUCACCUGG | 31041 | Breast tumor |
| 4721 | GUGACGGUGG | | AGCCCUCA | | |
| hsa-miR- | ACCUGCCAGCACC | 29000 | CUGCAGGGAGGUGC | 31042 | Breast tumor |
| 4722-3p | UCCCUGCAG | | UGGCAGGU | | |
| hsa-miR- | GGCAGGAGGGCU | 29001 | CAACCUGGCACAGC | 31043 | Breast tumor |
| 4722-5p | GUGCCAGGUUG | | CCUCCUGCC | | |
| hsa-miR- | CCCUCUCUGGCUC | 29002 | UUUGGGGAGGAGCC | 31044 | Breast tumor |
| 4723-3 | CUCCCCAAA | | AGAGAGGG | | |
| hsa-miR- | UGGGGGAGCCAU | 29003 | UGCUCUUAUCUCAU | 31045 | Breast tumor |
| 4723-5p | GAGAUAAGAGCA | | GGCUCCCCCA | | |
| hsa-miR- | GUACCUUCUGGU | 29004 | ACUAGCUGAACCAG | 31046 | Breast tumor |
| 4724-3p | UCAGCUAGU | | AAGGUAC | | |
| hsa-miR- | AACUGAACCAGG | 29005 | CGAAGCUCACUCCU | 31047 | Breast tumor |
| 4724-5p | AGUGAGCUUCG | | GGUUCAGUU | | |
| hsa-miR- | UGGGGAAGGCGU | 29006 | CCCGACACUGACGC | 31048 | Breast tumor |
| 4725-3p | CAGUGUCGGG | | CUUCCCCA | | |
| hsa-miR- | AGACCCUGCAGCC | 29007 | GGUGGGAAGGCUGC | 31049 | Breast tumor |
| 4725-5p | UUCCCACC | | AGGGUCU | | |
| hsa-miR- | ACCCAGGUUCCCU | 29008 | UGCGGCCAGAGGGA | 31050 | Breast tumor |
| 4726-3p | CUGGCCGCA | | ACCUGGGU | | |
| hsa-miR- | AGGGCCAGAGGA | 29009 | CCACUCCAGGCUCC | 31051 | Breast tumor |
| 4726-5p | GCCUGGAGUGG | | UCUGGCCCU | | |
| hsa-miR- | AUAGUGGGAAGC | 29010 | GAAUCUGCCAGCUU | 31052 | Breast tumor |
| 4727-3p | UGGCAGAUUC | | CCCACUAU | | |
| hsa-miR- | AUCUGCCAGCUUC | 29011 | CCACUGUGGAAGCU | 31053 | Breast tumor |
| 4727-5p | CACAGUGG | | GGCAGAU | | |
| hsa-miR- | CAUGCUGACCUCC | 29012 | CUGGGGCAGGAGGG | 31054 | Breast tumor |
| 4728-3p | CUCCUGCCCCAG | | AGGUCAGCAUG | | |
| hsa-miR- | UGGGAGGGGAGA | 29013 | UGCUUGCUGCCUCU | 31055 | Breast tumor |
| 4728-5p | GGCAGCAAGCA | | CCCCUCCCA | | |
| hsa-miR- | UCAUUUAUCUGU | 29014 | UAGCUUCCCAACAG | 31056 | Breast tumor |
| 4729 | UGGGAAGCUA | | AUAAAUGA | | |
| hsa-miR- | CUGGOGGAGCOCA | 29015 | UGGCAUGGAAUGGG | 31057 | Breast tumor |
| 4730 | UUCCAUGCCA | | CUCCGCCAG | | |
| hsa-miR- | CACACAAGUGGCC | 29016 | AGUGUUGGGGGCCA | 31058 | Breast tumor |
| 4731-3p | CCCAACACU | | CUUGUGUG | | |
| hsa-miR- | UGCUGGGGGCCAC | 29017 | CACACUCAUGUGGC | 31059 | Breast tumor |
| 4731-5p | AUGAGUGUG | | CCCCAGCA | | |
| hsa-miR- | GCCCUGACCUGUC | 29018 | CAGAACAGGACAGG | 31060 | Breast tumor |
| 4732-3p | CUGUUCUG | | UCAGGGC | | |
| hsa-miR- | UGUAGAGCAGGG | 29019 | AGCUUCCUGCUCCC | 31061 | Breast tumor |
| 4732-5p | AGCAGGAAGCU | | UGCUCUACA | | |
| hsa-miR- | CCACCAGGUCUAG | 29020 | AUCCCAAUGCUAGA | 31062 | Breast tumor |
| 4733-3p | CAUUGGGAU | | CCUGGUGG | | |
| hsa-miR- | AAUCCCAAUGCUA | 29021 | CACCGGGUCUAGCA | 31063 | Breast tumor |
| 4733-5p | GACCCGGUG | | UUGGGAUU | | |
| hsa-miR- | GCUGCGGGCUGCG | 29022 | CGCCCUGACCGCAG | 31064 | Breast tumor |
| 4734 | GUCAGGGCG | | CCCGCAGC | | |
| hsa-miR- | AAAGGUGCUCAA | 29023 | AUGUCUAAUUUGAG | 31065 | Breast tumor |
| 4735-3p | AUUAGACAU | | CACCUUU | | |
| hsa-miR- | CCUAAUUUGAAC | 29024 | UACCGAAGGUGUUC | 31066 | Breast tumor |
| 4735-5p | ACCUUCGGUA | | AAAUUAGG | | |
| hsa-miR- | AGGCAGGUUAUC | 29025 | CAGCCCAGAUAACC | 31067 | Breast tumor |
| 4736 | UGGGCUG | | UGCCU | | |
| hsa-miR- | AUGCGAGGAUGC | 29026 | CACUGUCAGCAUCC | 31068 | Breast tumor |
| 4737 | UGACAGUG | | UCGCAU | | |
| hsa-miR- | UGAAACUGGAGC | 29027 | UCCUCCAGGCGCUC | 31069 | Breast tumor |
| 4738-3p | GCCUGGAGGA | | CAGUUUCA | | |
| hsa-miR- | ACCAGCGCGUUUU | 29028 | AUGAAACUGAAAAC | 31070 | Breast tumor |
| 4738-5p | CAGUUUCAU | | GCGCUGGU | | |
| hsa-miR- | AAGGGAGGAGGA | 29029 | AGGGCCCCUCCGCU | 31071 | Breast tumor |
| 4739 | GCGGAGGGGCCCU | | CCUCCUCCCUU | | |
| hsa-miR- | GCCCGAGAGGAUC | 29030 | GCAGGGACGGAUCC | 31072 | Breast tumor |
| 4740-3p | CGUCCCUGC | | UCUCGGGC | | |
| hsa-miR- | AGGACUGAUCCUC | 29031 | CCUGCCCGAGAGGA | 31073 | Breast tumor |
| 4740-5p | UCGGGCAGG | | UCAGUCCU | | |
| hsa-miR- | UCAGGCAAAGGG | 29032 | UCUGUAAAUAUCCC | 31074 | Breast tumor |
| 4742-5p | AUAUUUACAGA | | UUUGCCUGA | | |
| hsa-miR- | UUUCUGUCUUUU | 29033 | CUGGACCAGAAAAG | 31075 | Breast tumor |
| 4743-3p | CUGGUCCAG | | ACAGAAA | | |
| hsa-miR- | UGGCCGGAUGGG | 29034 | AUGCCUCCUGUCCC | 31076 | Breast tumor |
| 4743-5p | ACAGGAGGCAU | | AUCCGGCCA | | |
| hsa-miR- | UCUAAAGACUAG | 29035 | CAUAGCGAAGUCUA | 31077 | Breast tumor |
| 4744 | ACUUCGCUAUG | | GUCUUUAGA | | |
| hsa-miR- | UGGCCCGGCGACG | 29036 | GACCGUGAGACGUC | 31078 | Breast tumor |
| 4745-3p | UCUCACGGUC | | GCCGGGCCA | | |
| hsa-miR- | UGAGUGGGGCUC | 29037 | CGCCGUCCCGGGAG | 31079 | Breast tumor |
| 4745-5p | CCGGGACGGCG | | CCCCACUCA | | |
| hsa-miR- | AGCGGUGCUCCUG | 29038 | UCGGCCCGCAGGAG | 31080 | Breast tumor |
| 4746-3p | CGGGCCGA | | CACCGCU | | |
| hsa-miR- | CCGGUCCCAGGAG | 29039 | UCUGCAGGUUCUCC | 31081 | Breast tumor |
| 4746-5p | AACCUGCAGA | | UGGGACCGG | | |
| hsa-miR- | AAGGCCCGGGCUU | 29040 | CUGGGAGGAAAGCC | 31082 | Breast tumor |
| 4747-3p | UCCUCCCAG | | CGGGCCUU | | |
| hsa-miR- | AGGGAAGGAGGC | 29041 | CUAAGACCAAGCCU | 31083 | Breast tumor |
| 4747-5p | UUGGUCUUAG | | CCUUCCCU | | |
| hsa-miR- | GAGGUUUGGGGA | 29042 | AGCAAAUCCUCCCC | 31084 | Breast tumor |
| 4748 | GGAUUUGCU | | AAACCUC | | |
| hsa-miR- | CGCCCCUCCUGCC | 29043 | CUGUGGGGGCAGGA | 31085 | Breast tumor |
| 4749-3p | CCCACAG | | GGGGCG | | |
| hsa-miR- | UGCGGGGACAGG | 29044 | GAUGCCCUGGCCUG | 31086 | Breast tumor |
| 4749-5p | CCAGGGCAUC | | UCCCCGCA | | |
| hsa-miR- | CCUGACCCACCCC | 29045 | CUGCGGGAGGGGGU | 31087 | Breast tumor |
| 4750-3p | CUCCCGCAG | | GGGUCAGG | | |
| hsa-miR- | CUCGGGCGGAGG | 29046 | CACUCAACCACCUC | 31088 | Breast tumor |
| 4750-5p | UGGUUGAGUG | | CGCCCGAG | | |
| hsa-miR- | AGAGGACCCGUA | 29047 | CCUUCUAGCAGCUA | 31089 | Breast tumor |
| 4751 | GCUGCUAGAAGG | | CGGGUCCUCU | | |
| hsa-miR- | UUGUGGAUCUCA | 29048 | AGCACAUCCUUGAG | 31090 | Breast tumor |
| 4752 | AGGAUGUGCU | | AUCCACAA | | |
| hsa-miR- | UUCUCUUUCUUU | 29049 | ACACAAGGCUAAAG | 31091 | Breast tumor |
| 4753-3p | AGCCUUGUGU | | AAAGAGAA | | |
| hsa-miR- | CAAGGCCAAAGG | 29050 | CUGUUCUCUUCCUU | 31092 | Breast tumor |
| 4753-5p | AAGAGAACAG | | UGGCCUUG | | |
| hsa-miR- | AUGCGGACCUGG | 29051 | ACUCCGCUAACCCA | 31093 | Breast tumor |
| 4754 | GUUAGCGGAGU | | GGUCCGCAU | | |
| hsa-miR- | AGCCAGGCUCUGA | 29052 | ACUUUCCCUUCAGA | 31094 | Breast tumor |
| 4755-3p | AGGGAAAGU | | GCCUGGCU | | |
| hsa-miR- | UUUCCCUUCAGAG | 29053 | AAAGCCAGGCUCUG | 31095 | Breast tumor |
| 4755-5p | CCUGGCUUU | | AAGGGAAA | | |
| hsa-miR- | CCAGAGAUGGUU | 29054 | AUAGGAAGGCAACC | 31096 | Breast tumor |
| 4756-3p | GCCUUCCUAU | | AUCUCUGG | | |
| hsa-miR- | CAGGGAGGCGCUC | 29055 | AGCAGAGAGUGAGC | 31097 | Breast tumor |
| 4756-5p | ACUCUCUGCU | | GCCUCCCUG | | |
| hsa-miR- | CAUGACGUCACAG | 29056 | GCGAAGCCUCUGUG | 31098 | Breast tumor |
| 4757-3p | AGGCUUCGC | | ACGUCAUG | | |
| hsa-miR- | AGGCCUCUGUGAC | 29057 | ACACCGUGACGUCA | 31099 | Breast tumor |
| 4757-5p | GUCACGGUGU | | CAGAGGCCU | | |
| hsa-miR- | UGCCCCACCUGCU | 29058 | GAGGGUGGUCAGCA | 31100 | Breast tumor |
| 4758-3p | GACCACCCUC | | GGUGGGGCA | | |
| hsa-miR- | GUGAGUGGGAGC | 29059 | CAGCCCCACCGGCU | 31101 | Breast tumor |
| 4758-5p | CGGUGGGGCUG | | CCCACUCAC | | |
| hsa-miR- | UAGGACUAGAUG | 29060 | UAAUUCCAACAUCU | 31102 | Breast tumor |
| 4759 | UUGGAAUUA | | AGUCCUA | | |
| hsa-miR- | AAAUUCAUGUUC | 29061 | GGUUUAGAUUGAAC | 31103 | Breast tumor |
| 4760-3p | AAUCUAAACC | | AUGAAUUU | | |
| hsa-miR- | UUUAGAUUGAAC | 29062 | CUAACUUCAUGUUC | 31104 | Breast tumor |
| 4760-5p | AUGAAGUUAG | | AAUCUAAA | | |
| hsa-miR- | GAGGGCAUGCGC | 29063 | GGACAAAGUGCGCA | 31105 | Breast tumor |
| 4761-3p | ACUUUGUCC | | UGCCCUC | | |
| hsa-miR- | ACAAGGUGUGCA | 29064 | GGUCAGGCAUGCAC | 31106 | Breast tumor |
| 4761-5p | UGCCUGACC | | ACCUUGU | | |
| hsa-miR- | CUUCUGAUCAAG | 29065 | CACCACAAAUCUUG | 31107 | Breast tumor |
| 4762-3p | AUUUGUGGUG | | AUCAGAAG | | |
| hsa-miR- | CCAAAUCUUGAUC | 29066 | AGGCUUCUGAUCAA | 31108 | Breast tumor |
| 4762-5p | AGAAGCCU | | GAUUUGG | | |
| hsa-miR- | AGGCAGGGGCUG | 29067 | CCCGCCCAGCACCA | 31109 | Breast tumor |
| 4763-3p | GUGCUGGGCGGG | | GCCCCUGCCU | | |
| hsa-miR- | CGCCUGCCCAGCC | 29068 | AGCAGGAGGGCUGG | 31110 | Breast tumor |
| 4763-5p | CUCCUGCU | | GCAGGCG | | |
| hsa-miR- | UUAACUCCUUUCA | 29069 | CCAUGGGUGUGAAA | 31111 | Breast tumor |
| 4764-3p | CACCCAUGG | | GGAGUUAA | | |
| hsa-miR- | UGGAUGUGGAAG | 29070 | AGAUAACUCCUUCC | 31112 | Breast tumor |
| 4764-5p | GAGUUAUCU | | ACAUCCA | | |
| hsa-miR- | UGAGUGAUUGAU | 29071 | GAACAUAGCUAUCA | 31113 | Breast tumor |
| 4765 | AGCUAUGUUC | | AUCACUCA | | |
| hsa-miR- | AUAGCAAUUGCU | 29072 | UUCCAAAAGAGCAA | 31114 | Breast tumor |
| 4766-3p | CUUUUGGAA | | UUGCUAU | | |
| hsa-miR- | UCUGAAAGAGCA | 29073 | AACACCAACUGCUC | 31115 | Breast tumor |
| 4766-5p | GUUGGUGUU | | UUUCAGA | | |
| hsa-miR- | CGCGGGCGCUCCU | 29074 | GGCGGCGGCCAGGA | 31116 | Breast tumor |
| 4767 | GGCCGCCGCC | | GCGCCCGCG | | |
| hsa-miR- | CCAGGAGAUCCAG | 29075 | AUUCUCUCUGGAUC | 31117 | Breast tumor |
| 4768-3p | AGAGAAU | | UCCUGG | | |
| hsa-miR- | AUUCUCUCUGGA | 29076 | AUCCAUGGGAUCCA | 31118 | Breast tumor |
| 4768-5p | UCCCAUGGAU | | GAGAGAAU | | |
| hsa-miR- | UCUGCCAUCCUCC | 29077 | GUAGGGGAGGGAGG | 31119 | Breast tumor |
| 4769-3p | CUCCCCUAC | | AUGGCAGA | | |
| hsa-miR- | GGUGGGAUGGAG | 29078 | CUCAUACCUUCUCU | 31120 | Breast tumor |
| 4769-5p | AGAAGGUAUGAG | | CCAUCCCACC | | |
| hsa-miR- | UGAGAUGACACU | 29079 | AGCUACAGUGUCAU | 31121 | Breast tumor |
| 4770 | GUAGCU | | CUCA | | |
| hsa-miR- | AGCAGACUUGACC | 29080 | UAAUUGUAGGUCAA | 31122 | Breast tumor |
| 4771 | UACAAUUA | | GUCUGCU | | |
| hsa-miR- | CAGAACAGGAGC | 29081 | GCCUUUCUAUGCUC | 31123 | Breast tumor |
| 4773 | AUAGAAAGGC | | CUGUUCUG | | |
| hsa-miR- | UUAAUUUUUUGU | 29082 | AGUGACCGAAACAA | 31124 | Breast tumor |
| 4775 | UUCGGUCACU | | AAAAUUAA | | |
| hsa-miR- | CUUGCCAUCCUGG | 29083 | AUGCAGUGGACCAG | 31125 | Breast tumor |
| 4776-3p | UCCACUGCAU | | GAUGGCAAG | | |
| hsa-miR- | GUGGACCAGGAU | 29084 | AGCCCUUGCCAUCC | 31126 | Breast tumor |
| 4776-5p | GGCAAGGGCU | | UGGUCCAC | | |
| hsa-miR- | AUACCUCAUCUAG | 29085 | UACAGCAUUCUAGA | 31127 | Breast tumor |
| 4777-3p | AAUGCUGUA | | UGAGGUAU | | |
| hsa-miR- | UUCUAGAUGAGA | 29086 | UAUAUAUAUCUCUC | 31128 | Breast tumor |
| 4777-5p | GAUAUAUAUA | | AUCUAGAA | | |
| hsa-miR- | UCUUCUUCCUUUG | 29087 | UCAACUCUGCAAAG | 31129 | Breast tumor |
| 4778-3p | CAGAGUUGA | | GAAGAAGA | | |
| hsa-miR- | AAUUCUGUAAAG | 29088 | CCUCUUCUUCCUUU | 31130 | Breast tumor |
| 4778-5p | GAAGAAGAGG | | ACAGAAUU | | |
| hsa-miR- | UAGGAGGGAAUA | 29089 | CUGCUUUUACUAUU | 31131 | Breast tumor |
| 4779 | GUAAAAGCAG | | CCCUCCUA | | |
| hsa-miR- | ACCCUUGAGCCUG | 29090 | GCUAGGGAUCAGGC | 31132 | Breast tumor |
| 4780 | AUCCCUAGC | | UCAAGGGU | | |
| hsa-miR- | AAUGUUGGAAUC | 29091 | CUCUAGCGAGGAUU | 31133 | Breast tumor |
| 4781-3p | CUCGCUAGAG | | CCAACAUU | | |
| hsa-miR- | UAGCGGGGAUUC | 29092 | CCAAUAUUGGAAUC | 31134 | Breast tumor |
| 4781-5p | CAAUAUUGG | | CCCGCUA | | |
| hsa-miR- | UGAUUGUCUUCA | 29093 | GUUCUAGAUAUGAA | 31135 | Breast tumor |
| 4782-3p | UAUCUAGAAC | | GACAAUCA | | |
| hsa-miR- | UUCUGGAUAUGA | 29094 | UUGAUUGUCUUCAU | 31136 | Breast tumor |
| 4782-5p | AGACAAUCAA | | AUCCAGAA | | |
| hsa-miR- | CCCCGGUGUUGGG | 29095 | GCAGACGCGCCCCA | 31137 | Breast tumor |
| 4783-3p | GCGCGUCUGC | | ACACCGGGG | | |
| hsa-miR- | GGCGCGCCCAGCU | 29096 | AGCCCGGGAGCUGG | 31138 | Breast tumor |
| 4783-5p | CCCGGGCU | | GCGCGCC | | |
| hsa-miR- | UGAGGAGAUGCU | 29097 | UCAGUCCCAGCAUC | 31139 | Breast tumor |
| 4784 | GGGACUGA | | UCCUCA | | |
| hsa-miR- | AGAGUCGGCGAC | 29098 | GCUGGCGGCGUCGC | 31140 | Breast tumor |
| 4785 | GCCGCCAGC | | CGACUCU | | |
| hsa-miR- | UGAAGCCAGCUCU | 29099 | GCCCAGACCAGAGC | 31141 | Breast tumor |
| 4786-3p | GGUCUGGGC | | UGGCUUCA | | |
| hsa-miR- | UGAGACCAGGAC | 29100 | GGUGCAUCCAGUCC | 31142 | Breast tumor |
| 4786-5p | UGGAUGCACC | | UGGUCUCA | | |
| hsa-miR- | GAUGCGCCGCCCA | 29101 | GCGCGGGGCAGUGG | 31143 | Breast tumor |
| 4787-3p | CUGCCCCGCGC | | GCGGCGCAUC | | |
| hsa-miR- | GCGGGGGUGGCG | 29102 | GGGAUGCCGCCGCC | 31144 | Breast tumor |
| 4787-5p | GCGGCAUCCC | | ACCCCCGC | | |
| hsa-miR- | UUACGGACCAGCU | 29103 | GCCUCCCUUAGCUG | 31145 | Breast tumor |
| 4788 | AAGGGAGGC | | GUCCGUAA | | |
| hsa-miR- | CACACAUAGCAGG | 29104 | UAUAUACACCUGCU | 31146 | Breast tumor |
| 4789-3p | UGUAUAUA | | AUGUGUG | | |
| hsa-miR- | GUAUACACCUGA | 29105 | CAUACACAUAUCAG | 31147 | Breast tumor |
| 4789-5p | UAUGUGUAUG | | GUGUAUAC | | |
| hsa-miR- | UGAAUGGUAAAG | 29106 | UGUGACAUCGCUUU | 31148 | Breast tumor |
| 4790-3p | CGAUGUCACA | | ACCAUUCA | | |
| hsa-miR- | AUCGCUUUACCAU | 29107 | AACAUGAAUGGUAA | 31149 | Breast tumor |
| 4790-5p | UCAUGUU | | AGCGAU | | |
| hsa-miR- | UGGAUAUGAUGA | 29108 | UUUCAGUCAUCAUA | 31150 | Breast tumor |
| 4791 | CUGAAA | | UCCA | | |
| hsa-miR- | CGGUGAGCGCUCG | 29109 | GCCAGCGAGCGCUC | 31151 | Breast tumor |
| 4792 | CUGGC | | ACCG | | |
| hsa-miR- | UCUGCACUGUGA | 29110 | AGCCAGCCAACUCA | 31152 | Breast tumor |
| 4793-3p | GUUGGCUGGCU | | CAGUGCAGA | | |
| hsa-miR- | ACAUCCUGCUCCA | 29111 | CCUCUGCCCUGUGG | 31153 | Breast tumor |
| 4793-5p | CAGGGCAGAGG | | AGCAGGAUGU | | |
| hsa-miR- | UCUGGCUAUCUCA | 29112 | ACAGUCUCGUGAGA | 31154 | Breast tumor |
| 4794 | CGAGACUGU | | UAGCCAGA | | |
| hsa-miR- | AUAUUAUUAGCC | 29113 | AUCCAGAAGUGGCU | 31155 | Breast tumor |
| 4795-3p | ACUUCUGGAU | | AAUAAUAU | | |
| hsa-miR- | AGAAGUGGCUAA | 29114 | UCAAUAUUAUUAGC | 31156 | Breast tumor |
| 4795-5p | UAAUAUUGA | | CACUUCU | | |
| hsa-miR- | UAAAGUGGCAGA | 29115 | GUGUCUAUACUCUG | 31157 | Breast tumor |
| 4796-3p | GUAUAGACAC | | CCACUUUA | | |
| hsa-miR- | UGUCUAUACUCU | 29116 | GUAAAGUGACAGAG | 31158 | Breast tumor |
| 4796-5p | GUCACUUUAC | | UAUAGACA | | |
| hsa-miR- | UCUCAGUAAGUG | 29117 | ACAGAGUGCCACUU | 31159 | Breast tumor |
| 4797-3p | GCACUCUGU | | ACUGAGA | | |
| hsa-miR- | GACAGAGUGCCAC | 29118 | UUCAGUAAGUGGCA | 31160 | Breast tumor |
| 4797-5p | UUACUGAA | | CUCUGUC | | |
| hsa-miR- | AACUCACGAAGU | 29119 | ACUUCGGUAUACUU | 31161 | Breast tumor |
| 4798-3p | AUACCGAAGU | | CGUGAGUU | | |
| hsa-miR- | UUCGGUAUACUU | 29120 | CCAAUUCACAAAGU | 31162 | Breast tumor |
| 4798-5p | UGUGAAUUGG | | AUACCGAA | | |
| hsa-miR- | ACUGGCAUGCUGC | 29121 | UAUAUAAAUGCAGC | 31163 | Breast tumor |
| 4799-3p | AUUUAUAUA | | AUGCCAGU | | |
| hsa-miR- | AUCUAAAUGCAG | 29122 | GACUGGCAUGCUGC | 31164 | Breast tumor |
| 4799-5p | CAUGCCAGUC | | AUUUAGAU | | |
| hsa-miR- | CAUCCGUCCGUCU | 29123 | GUGGACAGACGGAC | 31165 | Breast tumor |
| 4800-3p | GUCCAC | | GGAUG | | |
| hsa-miR- | AGUGGACCGAGG | 29124 | UCCUUCCUUCCUCG | 31166 | Breast tumor |
| 4800-5p | AAGGAAGGA | | GUCCACU | | |
| hsa-miR- | UACACAAGAAAA | 29125 | UGAGCCUUGGUUUU | 31167 | Breast tumor |
| 4801 | CCAAGGCUCA | | CUUGUGUA | | |
| hsa-miR- | UAACAUAAUAGU | 29126 | UCAAUCCACACUAU | 31168 | Breast tumor |
| 4803 | GUGGAUUGA | | UAUGUUA | | |
| hsa-miR- | UGCUUAACCUUGC | 29127 | UUUCGAGGGCAAGG | 31169 | Breast tumor |
| 4804-3p | CCUCGAAA | | UUAAGCA | | |
| hsa-miR- | UUGGACGGUAAG | 29128 | UUGCUUAACCUUAC | 31170 | Breast tumor |
| 4804-5p | GUUAAGCAA | | CGUCCAA | | |
| hsa-miR- | AAAAGCAUCAGG | 29129 | UGGGUACUUCCUGA | 31171 | Breast tumor and B |
| 4422 | AAGUACCCA | | UGCUUUU | | cells |
| hsa-miR- | GCAGGACAGGCA | 29130 | AUCCACUUCUGCCU | 31172 | Breast tumor and B |
| 4436a | GAAGUGGAU | | GUCCUGC | | cells |
| hsa-miR- | CAGGGCUGGCAG | 29131 | ACCCAUGUCACUGC | 31173 | Breast tumor and B |
| 4446-3p | UGACAUGGGU | | CAGCCCUG | | cells |
| hsa-miR- | AUUUCCCUGCCAU | 29132 | GCCAAGGGAAUGGC | 31174 | Breast tumor and B |
| 4446-5p | UCCCUUGGC | | AGGGAAAU | | cells |
| hsa-miR- | UGGCGGCGGUAG | 29133 | AAGCCCAUAACUAC | 31175 | Breast tumor and B |
| 4467 | UUAUGGGCUU | | CGCCGCCA | | cells |
| hsa-miR- | GCUCCCUCUAGGG | 29134 | UCCGAGCGACCCUA | 31176 | Breast tumor and B |
| 4469 | UCGCUCGGA | | GAGGGAGC | | cells |
| hsa-miR- | UGGGAACUUAGU | 29135 | UUAAACCUCUACUA | 31177 | Breast tumor and B |
| 4471 | AGAGGUUUAA | | AGUUCCCA | | cells |
| hsa-miR- | UGGGGCUAGUGA | 29136 | CGUCCUGCAUCACU | 31178 | Breast tumor and B |
| 4489 | UGCAGGACG | | AGCCCCA | | cells |
| hsa-miR- | GCUGACAGCAGG | 29137 | AGCGGCCAGCCCUG | 31179 | Breast tumor and B |
| 4526 | GCUGGCCGCU | | CUGUCAGC | | cells |
| hsa-miR- | AUUGGACUGCUG | 29138 | ACGGGCCAUCAGCA | 31180 | Breast tumor and B |
| 4529-3p | AUGGCCCGU | | GUCCAAU | | cells |
| hsa-miR- | AGGCCAUCAGCAG | 29139 | UUCAUUGGACUGCU | 31181 | Breast tumor and B |
| 4529-5p | UCCAAUGAA | | GAUGGCCU | | cells |
| hsa-miR- | UUGGACAGAAAA | 29140 | UUCCUGCGUGUUUU | 31182 | Breast tumor and B |
| 4520a-3p | CACGCAGGAA | | CUGUCCAA | | cells, skin |
| | | | | | (psoriasis) |
| hsa-miR- | CCUGCGUGUUUUC | 29141 | UUGGACAGAAAACA | 31183 | Breast tumor and B |
| 4520a-5p | UGUCCAA | | CGCAGG | | cells, skin |
| | | | | | (psoriasis) |
| hsa-miR- | UGAGACAGGCUU | 29142 | AUAGCAGCAUAAGC | 31184 | Breast tumor and B |
| 4524a-3p | AUGCUGCUAU | | CUGUCUCA | | cells, skin |
| | | | | | (psoriasis) |
| hsa-miR- | AUAGCAGCAUGA | 29143 | UGAGACAGGUUCAU | 31185 | Breast tumor and B |
| 4524a-5p | ACCUGUCUCA | | GCUGCUAU | | cells, skin |
| | | | | | (psoriasis) |
| hsa-miR- | GAGACAGGUUCA | 29144 | UAGCAGCAUGAACC | 31186 | Breast tumor and B |
| 4524b-3p | UGCUGCUA | | UGUCUC | | cells, skin |
| | | | | | (psoriasis) |
| hsa-miR- | AUAGCAGCAUAA | 29145 | GAGACAGGCUUAUG | 31187 | Breast tumor and B |
| 4524b-Sp | GCCUGUCUC | | CUGCUAU | | cells, skin |
| | | | | | (psoriasis) |
| hsa-miR- | UGUGUGGAUCCU | 29146 | UGCCUCCUCCAGGA | 31188 | Breast tumor and |
| 3911 | GGAGGAGGCA | | UCCACACA | | female reproductive |
| | | | | | tract |
| hsa-miR- | AGACAUCAAGAU | 29147 | UUUGGGACUGAUCU | 31189 | Breast tumor and |
| 3913-3p | CAGUCCCAAA | | UGAUGUCU | | female reproductive |
| | | | | | tract |
| hsa-miR- | UUUGGGACUGAU | 29148 | AGACAUCAAGAUCA | 31190 | Breast tumor and |
| 3913-5p | CUUGAUGUCU | | GUCCCAAA | | female reproductive |
| | | | | | tract |
| hsa-miR- | AAGGAACCAGAA | 29149 | ACUUCUCAUUUUCU | 31191 | Breast tumor and |
| 3914 | AAUGAGAAGU | | GGUUCCUU | | female reproductive |
| | | | | | tract |
| hsa-miR- | UCUGGCCUUGACU | 29150 | AAAGAGUCAAGUCA | 31192 | Breast tumor and |
| 3922-3p | UGACUCUUU | | AGGCCAGA | | female reproductive |
| | | | | | tract |
| hsa-miR- | UCAAGGCCAGAG | 29151 | UGCUGUGGGACCUC | 31193 | Breast tumor and |
| 3922-5p | GUCCCACAGCA | | UGGCCUUGA | | female reproductive |
| | | | | | tract |
| hsa-miR- | ACUCCAGUUUUA | 29152 | CAAGAGAACUAAAA | 31194 | Breast tumor and |
| 3925-3p | GUUCUCUUG | | CUGGAGU | | female reproductive |
| | | | | | tract |
| hsa-miR- | AAGAGAACUGAA | 29153 | AGGCUCCACUUUCA | 31195 | Breast tumor and |
| 3925-5p | AGUGGAGCCU | | GUUCUCUU | | female reproductive |
| | | | | | tract |
| hsa-miR- | UAAGGGGUGUAU | 29154 | UGCAUCUGCCAUAC | 31196 | Breast tumor and |
| 3936 | GGCAGAUGCA | | ACCCCUUA | | lymphoblastic |
| | | | | | leukemia |
| hsa-miR- | UUUCAGAUAACA | 29155 | AUGUAAUACUGUUA | 31197 | Breast tumor and |
| 3942-3p | GUAUUACAU | | UCUGAAA | | lymphoblastic |
| | | | | | leukemia |
| hsa-miR- | AAGCAAUACUGU | 29156 | AUUUCAGGUAACAG | 31198 | Breast tumor and |
| 3942-5p | UACCUGAAAU | | UAUUGCUU | | lymphoblastic |
| | | | | | leukemia |
| hsa-miR- | UACUAACUGCAG | 29157 | UCACUUGAAUCUGC | 31199 | Breast tumor and |
| 4637 | AUUCAAGUGA | | AGUUAGUA | | lymphoblastic |
| | | | | | leukemia |
| hsa-miR- | AUUGCCUAACAU | 29158 | UUCUGGCACAUGUU | 31200 | Breast tumor and |
| 4774-3p | GUGCCAGAA | | AGGCAAU | | Lymphoblastic |
| | | | | | leukemia |
| hsa-miR- | UCUGGUAUGUAG | 29159 | UUAUUACCUACUAC | 31201 | Breast tumor and |
| 4774-5p | UAGGUAAUAA | | AUACCAGA | | Lymphoblastic |
| | | | | | leukemia |
| hsa-miR- | AGUUGCCUUUUU | 29160 | GCAUGGGAACAAAA | 31202 | Breast tumor B |
| 4423-5p | GUUCCCAUGC | | AGGCAACU | | cells and skin |
| | | | | | (psoriasis) |
| hsa-miR- | AUAGGCACCAAA | 29161 | UUGUUGCUUUUUGG | 31203 | Breast tumor, B |
| 4423-3p | AAGCAACAA | | UGCCUAU | | cells and skin |
| | | | | | (psoriasis) |
| hsa-miR- | CCUGCAACUUUGC | 29162 | UCUGAUCAGGCAAA | 31204 | Breast tumor, blood |
| 4772-3p | CUGAUCAGA | | GUUGCAGG | | mononuclear cells |
| hsa-miR- | UGAUCAGGCAAA | 29163 | AGUCUGCAAUUUUG | 31205 | Breast tumor, blood |
| 4772-5p | AUUGCAGACU | | CCUGAUCA | | mononuclear cells |
| hsa-miR- | UUGUGGCUGGUC | 29164 | UUAGCCUCAUGACC | 31206 | Breast tumor, |
| 4474-3p | AUGAGGCUAA | | AGCCACAA | | lymphoblastic |
| | | | | | leukemia and B |
| | | | | | cells |
| hsa-miR- | UUAGUCUCAUGA | 29165 | UGUGUCUGAUCAUG | 31207 | Breast tumor, |
| 4474-5p | UCAGACACA | | AGACUAA | | lymphoblastic |
| | | | | | leukemia and B |
| | | | | | cells |
| hsa-miR- | AAAGAUAGACAA | 29166 | AUUUAGCCAAUUGU | 31208 | Breast tumor, |
| 4662a-3p | UUGGCUAAAU | | CUAUCUUU | | psoriasis |
| hsa-miR- | UUAGCCAAUUGU | 29167 | CUAAAGAUGGACAA | 31209 | Breast tumor, |
| 4662a-5p | CCAUCUUUAG | | UUGGCUAA | | psoriasis |
| hsa-miR- | UCUGUGAGACCA | 29168 | AGUAGUUCUUUGGU | 31210 | Breast tumor, |
| 4677-3p | AAGAACUACU | | CUCACAGA | | psoriasis |
| hsa-miR- | UUGUUCUUUGGU | 29169 | UGGCUGAAAGACCA | 31211 | Breast tumor, |
| 4677-5p | CUUUCAGCCA | | AAGAACAA | | psoriasis |
| hsa-miR- | CGGGCUGUCCGGA | 29170 | AGCCGACCCCUCCG | 31212 | Breast tumor, |
| 4741 | GGGGUCGGCU | | GACAGCCCG | | psoriasis |
| hsa-miR- | UCUGUAUUCUCCU | 29171 | CUGCAGGCAAAGGA | 31213 | Breast tumor, |
| 4742-3p | UUGCCUGCAG | | GAAUACAGA | | psoriasis |
| hsa-miR- | UACAUGGAUGGA | 29172 | GCUUGAAGGUUUCC | 31214 | Breast tumor, |
| 4802-3p | AACCUUCAAGC | | AUCCAUGUA | | psoriasis |
| hsa-miR- | UAUGGAGGUUCU | 29173 | AACAUGGUCUAGAA | 31215 | Breast tumor, |
| 4802-5p | AGACCAUGUU | | CCUCCAUA | | psoriasis |
| hsa-miR- | GGGACCAUCCUGC | 29174 | CCACAGCAGGCAGG | 31216 | Breast tumors |
| 3619-3p | CUGCUGUGG | | AUGGUCCC | | |
| hsa-miR- | UCAGCAGGCAGGC | 29175 | GCUGCACCAGCCUG | 31217 | Breast tumors |
| 3619-5p | UGGUGCAGC | | CCUGCUGA | | |
| hsa-miR- | UCACCUGACCUCC | 29176 | ACAGGCAUGGGAGG | 31218 | Breast tumors |
| 3622a-3p | CAUGCCUGU | | UCAGGUGA | | |
| hsa-miR- | CAGGCACGGGAGC | 29177 | CUCACCUGAGCUCC | 31219 | Breast tumors |
| 3622a-5p | UCAGGUGAG | | CGUGCCUG | | |
| hsa-miR- | UGAGUGUUGUCU | 29178 | UGCCCUCGUAGACA | 31220 | Breast tumors |
| 3659 | ACGAGGGCA | | ACACUCA | | |
| hsa-miR- | ACUGACAGGAGA | 29179 | UCAAAAUGCUCUCC | 31221 | Breast tumors |
| 3660 | GCAUUUUGA | | UGUCAGU | | |
| hsa-miR- | UGACCUGGGACUC | 29180 | CAGCUGUCCGAGUC | 31222 | Breast tumors |
| 3661 | GGACAGCUG | | CCAGGUCA | | |
| hsa-miR- | UCUCAGGAGUAA | 29181 | AACUCUGUCUUUAC | 31223 | Breast tumors |
| 3664-3p | AGACAGAGUU | | UCCUGAGA | | |
| hsa-miR- | AACUCUGUCUUCA | 29182 | ACUCAUGAGUGAAG | 31224 | Breast tumors |
| 3664-5p | CUCAUGAGU | | ACAGAGUU | | |
| hsa-miR- | UGGGGCGGAGCU | 29183 | GGCCUCCGGAAGCU | 31225 | Breast tunor |
| 3180-3p | UCCGGAGGCC | | CCGCCCCA | | |
| hsa-miR- | CAAUCAGCAAGU | 29184 | AGGGCAGUAUACUU | 31226 | Breast, myeloid |
| 34a-3p | AUACUGCCCU | | GCUGAUUG | | cells, ciliated |
| | | | | | epithelial cells |
| hsa-miR- | UGGCAGUGUCUU | 29185 | ACAACCAGCUAAGA | 31227 | Breast, myeloid |
| 34a-5p | AGCUGGUUGU | | CACUGCCA | | cells, ciliated |
| | | | | | epithelial cells |
| hsa-miR- | CCAAUAUUGGCU | 29186 | GGAGCAGCACAGCC | 31228 | Breast, pancreas |
| 195-3p | GUGCUGCUCC | | AAUAUUGG | | (islet) |
| hsa-miR- | UAGCAGCACAGA | 29187 | GCCAAUAUUUCUGU | 31229 | Breast, pancreas |
| 195-5p | AAUAUUGGC | | GCUGCUA | | (islet) |
| hsa-miR- | UAAUUUUAUGUA | 29188 | ACUAGCUUAUACAU | 31230 | Cardiomyocytes |
| 590-3p | UAAGCUAGU | | AAAAUUA | | |
| hsa-miR- | GAGCUUAUUCAU | 29189 | CUGCACUUUUAUGA | 31231 | Cardiomyocytes |
| 590-5p | AAAAGUGCAG | | AUAAGCUC | | |
| hsa-miR- | AAGACGGGAGGA | 29190 | CUCCCUUCUUUCCU | 31232 | Cartilage |
| 483-5p | AAGAAGGGAG | | CCCGUCUU | | (chondrocyte), fetal |
| | | | | | brain |
| hsa-miR- | CAGUGGUUUUAC | 29191 | CUACCAUAGGGUAA | 31233 | Cartilage |
| 140-5p | CCUAUGGUAG | | AACCACUG | | (chondrocytes) |
| hsa-miR- | AUUCUAAUUUCU | 29192 | AAAGACGUGGAGAA | 31234 | Cartilage/ |
| 576-5p | CCACGUCUUU | | AUUAGAAU | | chondrocyte |
| hsa-miR- | AACCAGCACCCCA | 29193 | GUCCAAAGUUGGGG | 31235 | Cartilage/ |
| 634 | ACUUUGGAC | | UGCUGGUU | | chondrocyte |
| hsa-miR- | AAAGACAUAGGA | 29194 | GAGGUGACUCUAUC | 31236 | Cartilage/ |
| 641 | UAGAGUCACCUC | | CUAUGUCUUU | | chondrocyte |
| hsa-miR- | UAACUGGUUGAA | 29195 | GGUUCAGUUGUUCA | 31237 | Cartilage/chondroc |
| 582-3p | CAACUGAACC | | ACCAGUUA | | yte |
| hsa-miR- | CGUGCCACCCUUU | 29196 | CUGGGGAAAAGGGU | 31238 | Cartilage/chondroc |
| 1227-3p | UCCCCAG | | GGCACG | | vtes |
| hsa-miR- | GUGGGGCCAGGC | 29197 | CCACCGCCUGGCCC | 31239 | Cartilage/chondroc |
| 1227-5p | GGUGG | | CAC | | vtes |
| hsa-miR- | AAAAGCUGGGUU | 29198 | UUGCCCUCUCAACC | 31240 | Central nervous |
| 320b | GAGAGGGCAA | | CAGCUUUU | | system |
| hsa-miR- | GGUCCAGAGGGG | 29199 | GAACCUAUCUCCCC | 31241 | Central nervous |
| 198 | AGAUAGGUUC | | UCUGGACC | | system(CNS) |
| hsa-miR- | UAGCCUUCAGAUC | 29200 | AAAACACCAAGAUC | 31242 | Cervical and breast |
| 3614-3p | UUGGUGUUUU | | UGAAGGCUA | | tumors |
| hsa-miR- | CCACUUGGAUCUG | 29201 | GGGCAGCCUUCAGA | 31243 | Cervical and breast |
| 3614-5p | AAGGCUGCCC | | UCCAAGUGG | | tumors |
| hsa-miR- | UGUGCGCAGGGA | 29202 | GGGAGAGGUCUCCC | 31244 | Cervical cancer |
| 933 | GACCUCUCCC | | UGCGCACA | | |
| hsa-miR- | UGUCUACUACUG | 29203 | CCAGUGUCUCCAGU | 31245 | Cervical cancer |
| 934 | GAGACACUGG | | AGUAGACA | | |
| hsa-miR- | AAGGCAGGGCCCC | 29204 | GGGGAGCGGGGGCC | 31246 | Cervical cancer |
| 940 | CGCUCCCC | | CUGCCUU | | |
| hsa-miR- | CUGACUGUUGCCG | 29205 | CUGGAGGACGGCAA | 31247 | Cervical cancer |
| 943 | UCCUCCAG | | CAGUCAG | | |
| hsa-miR- | AAAAACCACAAU | 29206 | UGGUGCAAAAGUAA | 31248 | Cervical tumor |
| 548aa | UACUUUUGCACCA | | UUGUGGUUUUU | | |
| hsa-miR- | CAAAAACCGCAAU | 29207 | UGCAAAAGUAAUUG | 31249 | Cervical tumor |
| 548z | UACUUUUGCA | | CGGUUUUUG | | |
| hsa-miR- | AUGUGCCUGAGG | 29208 | UGUCUUACUCCCUC | 31250 | Cervical tumor |
| 550b-2-5p | GAGUAAGACA | | AGGCACAU | | |
| hsa-miR- | UCUUACUCCCUCA | 29209 | CAGUGCCUGAGGGA | 31251 | Cervical tumor |
| 550b-3p | GGCACUG | | GUAAGA | | |
| hsa-miR- | AGACACAUUUGG | 29210 | GGGUCCCUCUCCAA | 31252 | Cervical tumor |
| 642b-3p | AGAGGGACCC | | AUGUGUCU | | |
| hsa-miR- | GGUUCCCUCUCCA | 29211 | AGACACAUUUGGAG | 31253 | Cervical tumor |
| 642b-5p | AAUGUGUCU | | AGGGAACC | | |
| hsa-miR- | AAAAUUUCUUUC | 29212 | CUAAGUAGUGAAAG | 31254 | Cervical tumors |
| 3606-3p | ACUACUUAG | | AAAUUUU | | |
| hsa-miR- | UUAGUGAAGGCU | 29213 | AAUUAAAAUAGCCU | 31255 | Cervical tumors |
| 3606-5p | AUUUUAAUU | | UCACUAA | | |
| hsa-miR- | ACUGUAAACGCU | 29214 | CAUCAGAAAGCGUU | 31256 | Cervical tumors |
| 3607-3p | UUCUGAUG | | UACAGU | | |
| hsa-miR- | GCAUGUGAUGAA | 29215 | ACUGAUUUGCUUCA | 31257 | Cervical tumors |
| 3607-5p | GCAAAUCAGU | | UCACAUGC | | |
| hsa-miR- | CAAAGUGAUGAG | 29216 | CAGCCAGUAUUACU | 31258 | Cervical tumors |
| 3609 | UAAUACUGGCUG | | CAUCACUUUG | | |
| hsa-miR- | GAAUCGGAAAGG | 29217 | CGGCGCCUCCUUUC | 31259 | Cervical tumors |
| 3610 | AGGCGCCG | | CGAUUC | | |
| hsa-miR- | UUGUGAAGAAAG | 29218 | UAAGAAUUUCUUUC | 31260 | Cervical tumors |
| 3611 | AAAUUCUUA | | UUCACAA | | |
| hsa-miR- | AGGAGGCAUCUU | 29219 | UCCAUUUCUCAAGA | 31261 | Cervical tumors |
| 3612 | GAGAAAUGGA | | UGCCUCCU | | |
| hsa-miR- | ACAAAAAAAAAA | 29220 | GAAGGGUUGGGCUU | 31262 | Cervical tumors |
| 3613-3p | GCCCAACCCUUC | | UUUUUUUUGU | | |
| hsa-miR- | UGUUGUACUUUU | 29221 | GAACAAAAAAAAAA | 31263 | Cervical tumors |
| 3613-5p | UUUUUUGUUC | | GUACAACA | | |
| hsa-miR- | UCUCUCGGCUCCU | 29222 | GAGCCGCGAGGAGC | 31264 | Cervical tumors |
| 3615 | CGCGGCUC | | CGAGAGA | | |
| hsa-miR- | CGAGGGCAUUUC | 29223 | GCCUGCAUCAUGAA | 31265 | Cervical tumors |
| 3616-3p | AUGAUGCAGGC | | AUGCCCUCG | | |
| hsa-miR- | AUGAAGUGCACU | 29224 | ACAUAUCAUGAGUG | 31266 | Cervical tumors |
| 3616-5p | CAUGAUAUGU | | CACUUCAU | | |
| hsa-miR- | UGUCUACAUUAA | 29225 | GCUCUUUUCAUUAA | 31267 | Cervical tumors |
| 3618 | UGAAAAGAGC | | UGUAGACA | | |
| hsa-miR- | UCACCCUGCAUCC | 29226 | CUGGGUGCGGGAUG | 31268 | Cervical tumors |
| 3620-3p | CGCACCCAG | | CAGGGUGA | | |
| hsa-miR- | GUGGGCUGGGCU | 29227 | GGCCCAGCCCAGCC | 31269 | Cervical tumors |
| 3620-5p | GGGCUGGGCC | | CAGCCCAC | | |
| hsa-miR- | CGCGGGUCGGGG | 29228 | CCUGCAGACCCCGA | 31270 | Cervical tumors |
| 3621 | UCUGCAGG | | CCCGCG | | |
| hsa-miR- | UCACCUGAGCUCC | 29229 | CAGGCACGGGAGCU | 31271 | Cervical tumors |
| 3622b-3p | CGUGCCUG | | CAGGUGA | | |
| hsa-miR- | AGGCAUGGGAGG | 29230 | UCACCUGACCUCCC | 31272 | Cervical tumors |
| 3622b-5p | UCAGGUGA | | AUGCCU | | |
| hsa-miR- | CAUCAGCACCCUA | 29231 | AGAAAGGACAUAGG | 31273 | Cervical tumors and |
| 3617-3p | UGUCCUUUCU | | GUGCUGAUG | | psoriasis |
| hsa-miR- | AAAGACAUAGUU | 29232 | CCCAUCUUGCAACU | 31274 | Cervical tumors and |
| 3617-5p | GCAAGAUGGG | | AUGUCUUU | | psoriasis |
| hsa-miR- | UUUGUGACCUGG | 29233 | GGUUAGUGGACCAG | 31275 | Cholesterol |
| 758-3p | UCCACUAACC | | GUCACAAA | | regulation and brain |
| hsa-miR- | GAUGGUUGACCA | 29234 | GUGUGCUCUCUGGU | 31276 | Cholesterol |
| 758-5p | GAGAGCACAC | | CAACCAUC | | regulation and brain |
| hsa-miR- | AAAAGCUGGGUU | 29235 | ACCCUCUCAACCCA | 31277 | Chondrocyte |
| 320c | GAGAGGGU | | GCUUUU | | |
| hsa-miR- | CACAAGGUAUUG | 29236 | AGGUAAUACCAAUA | 31278 | Chondrocyte |
| 624-3p | GUAUUACCU | | CCUUGUG | | |
| hsa-miR- | UAGUACCAGUACC | 29237 | UGAACACAAGGUAC | 31279 | Chondrocyte |
| 624-5p | UUGUGUUCA | | UGGUACUA | | |
| hsa-miR- | AGUAUUCUGUAC | 29238 | ACCUUCCCUGGUAC | 31280 | Chondrocytes |
| 630 | CAGGGAAGGU | | AGAAUACU | | |
| hsa-miR- | UGGCAGUGUAUU | 29239 | ACCAGCUAACAAUA | 31281 | Chondrocytes, |
| 449a | GUUAGCUGGU | | CACUGCCA | | ciliated epithelial |
| | | | | | cells |
| hsa-miR- | UAUACAAGGGCA | 29240 | ACAGAGAGCUUGCC | 31282 | Chondrogenesis, |
| 381-3p | AGCUCUCUGU | | CUUGUAUA | | lung, brain |
| hsa-miR- | AGCGAGGUUGCCC | 29241 | AUAUACAAAGGGCA | 31283 | Chondrogenesis, |
| 381-5p | UUUGUAUAU | | ACCUCGCU | | lung, brain |
| hsa-miR- | CAAUCACUAACUC | 29242 | AUGGCAGUGGAGUU | 31284 | Ciliated epithelial |
| 34b-3p | CACUGCCAU | | AGUGAUUG | | cells |
| hsa-miR- | UAGGCAGUGUCA | 29243 | CAAUCAGCUAAUGA | 31285 | Ciliated epithelial |
| 34b-5p | UUAGCUGAUUG | | CACUGCCUA | | cells |
| hsa-miR- | CAGCCACAACUAC | 29244 | AGUGGCAGGGUAGU | 31286 | Ciliated epithelial |
| 449b-3p | CCUGCCACU | | UGUGGCUG | | cells, other tissues |
| hsa-miR- | AGGCAGUGUAUU | 29245 | GCCAGCUAACAAUA | 31287 | Ciliated epithelial |
| 449b-5p | GUUAGCUGGC | | CACUGCCU | | cells, other tissues |
| hsa-miR- | AAUCACUAACCAC | 29246 | CCUGGCCGUGUGGU | 31288 | Ciliated epithelial |
| 34c-3p | ACGGCCAGG | | UAGUGAUU | | cells, placenta |
| hsa-miR- | AGGCAGUGUAGU | 29247 | GCAAUCAGCUAACU | 31289 | Ciliated epithelial |
| 34c-5p | UAGCUGAUUGC | | ACACUGCCU | | cells, placenta |
| hsa-miR- | GUCCGCUCGGCGG | 29248 | UGGGCCACCGCCGA | 31290 | Circulating |
| 572 | UGGCCCA | | GCGGAC | | microrna (in |
| | | | | | plasma) |
| hsa-miR- | GAACGCCUGUUCU | 29249 | CCACCUGGCAAGAA | 31291 | Circulating |
| 614 | UGCCAGGUGG | | CAGGCGUUC | | micrornas (in |
| | | | | | Plasma) |
| hsa-miR- | AAGUGUGCAGGG | 29250 | ACCAGUGCCCUGCA | 31292 | Circulating |
| 648 | CACUGGU | | CACUU | | micrornas (in |
| | | | | | Plasma) |
| hsa-miR- | AGGGGUGCUAUC | 29251 | UCAAUCACAGAUAG | 31293 | Circulating plasma |
| 342-5p | UGUGAUUGA | | CACCCCU | | |
| hsa-miR- | CUUUCAGUCAGA | 29252 | GCAGCAAACAUCUG | 31294 | CNS (prefrontal |
| 30d-3p | UGUUUGCUGC | | ACUGAAAG | | cortex |
| hsa-miR- | UGUAAACAUCCCC | 29253 | CUUCCAGUCGGGGA | 31295 | CNS (prefrontal |
| 30d-5p | GACUGGAAG | | UGUUUACA | | cortex, embryoid |
| | | | | | body cells |
| hsa-miR- | UGUAAACAUCCUC | 29254 | CUUCCAGUCGAGGA | 31296 | CNS(prefrontal |
| 30a-5p | GACUGGAAG | | UGUUUACA | | cortex), other |
| | | | | | tissues |
| hsa-miR- | AGAAGUAAUUGC | 29255 | UGGCAAAACCGCAA | 31297 | Colorectal |
| 548a | GGUUUUGCCA | | UUACUUCU | | micrornaome |
| hsa-miR- | CAAAACUGGCAA | 29256 | GCAAAAGUAAUUGC | 31298 | Colorectal |
| 548a-3p | UUACUUUUGC | | CAGUUUUG | | micrornaome |
| hsa-miR- | AAAAGUAAUUGC | 29257 | GGUAAAACUCGCAA | 31299 | Colorectal |
| 548a-5p | GAGUUUUACC | | UUACUUUU | | micrornaome |
| hsa-miR- | CAAGAACCUCAGU | 29258 | ACAAAAGCAACUGA | 31300 | Colorectal |
| 548b-3p | UGCUUUUGU | | GGUUCUUG | | micrornaome |
| hsa-miR- | CAAAAAUCUCAA | 29259 | GCAAAAGUAAUUGA | 31301 | Colorectal |
| 548c-3p | UUACUUUUGC | | GAUUUUUG | | micrornaome |
| hsa-miR- | CAAAAACCACAGU | 29260 | GCAAAAGAAACUGU | 31302 | Colorectal |
| 548d-3p | UUCUUUUGC | | GGUUUUUG | | micrornaome |
| hsa-miR- | AAAAGUAAUUGU | 29261 | GGCAAAAACCACAA | 31303 | Colorectal |
| 548d-5p | GGUUUUUGCC | | UUACUUUU | | micrornaome |
| hsa-miR- | UGACAACUAUGG | 29262 | AGAGCUCAUCCAUA | 31304 | Colorectal |
| 549a | AUGAGCUCU | | GUUGUCA | | micrornaome |
| hsa-miR- | AACAGGUGACUG | 29263 | UUGUCUAACCAGUC | 31305 | Colorectal |
| 552 | GUUAGACAA | | ACCUGUU | | micrornaome |
| hsa-miR- | AAAACGGUGAGA | 29264 | AAAACAAAAUCUCA | 31306 | Colorectal |
| 553 | UUUUGUUUU | | CCGUUUU | | micrornaome |
| hsa-miR- | GCUAGUCCUGACU | 29265 | ACUGGCUGAGUCAG | 31307 | Colorectal |
| 554 | CAGCCAGU | | GACUAGC | | micrornaome |
| hsa-miR- | AGGGUAAGCUGA | 29266 | AUCAGAGGUUCAGC | 31308 | Colorectal |
| 555 | ACCUCUGAU | | UUACCCU | | micrornaome |
| hsa-miR- | AUAUUACCAUUA | 29267 | AAAGAUGAGCUAAU | 31309 | Colorectal |
| 556-3p | GCUCAUCUUU | | GGUAAUAU | | micrornaome |
| hsa-miR- | GAUGAGCUCAUU | 29268 | CUCAUAUUACAAUG | 31310 | Colorectal |
| 556-5p | GUAAUAUGAG | | AGCUCAUC | | micrornaome |
| hsa-miR- | AGGUUGACAUAC | 29269 | GGGAAACGUAUGUC | 31311 | Colorectal |
| 563 | GUUUCCC | | AACCU | | micrornaome |
| hsa-miR- | AUGUAUAAAUGU | 29270 | GUGUGUAUACAUUU | 31312 | Colorectal |
| 568 | AUACACAC | | AUACAU | | micrornaome |
| hsa-miR- | CUGAAGUGAUGU | 29271 | CUGAUCAGUUACAC | 31313 | Colorectal |
| 573 | GUAACUGAUCAG | | AUCACUUCAG | | micrornaome |
| hsa-miR- | AAGAUGUGGAAA | 29272 | GAUUCCAAUUUUUC | 31314 | Colorectal |
| 576-3p | AAUUGGAAUC | | CACAUCUU | | micrornaome |
| hsa-miR- | UAGAUAAAAUAU | 29273 | CAGGUACCAAUAUU | 31315 | Colorectal |
| 577 | UGGUACCUG | | UUAUCUA | | micrornaome |
| hsa-miR- | CUUCUUGUGCUCU | 29274 | ACAAUCCUAGAGCA | 31316 | Colorectal |
| 578 | AGGAUUGU | | CAAGAAG | | micrornaome |
| hsa-miR- | UAUGCAUUGUAU | 29275 | GGACCUAAAAAUAC | 31317 | Colorectal |
| 586 | UUUUAGGUCC | | AAUGCAUA | | micrornaome |
| hsa-miR- | UUUCCAUAGGUG | 29276 | GUGACUCAUCACCU | 31318 | Colorectal |
| 587 | AUGAGUCAC | | AUGGAAA | | micrornaome |
| hsa-miR- | UUGGCCACAAUG | 29277 | GUUCUAACCCAUUG | 31319 | Colorectal |
| 588 | GGUUAGAAC | | UGGCCAA | | micrornaome |
| hsa-miR- | UGUGUCACUCGA | 29278 | ACAGUGGUCAUCGA | 31320 | Colorectal |
| 597 | UGACCACUGU | | GUGACACA | | micrornaome |
| hsa-miR- | ACUUACAGACAA | 29279 | GAGCAAGGCUCUUG | 31321 | Colorectal |
| 600 | GAGCCUUGCUC | | UCUGUAAGU | | micrornaome |
| hsa-miR- | AGGCUGCGGAAU | 29280 | GUCCUGAAUUCCGC | 31322 | Colorectal |
| 604 | UCAGGAC | | AGCCU | | micrornaome |
| hsa-miR- | UAAAUCCCAUGG | 29281 | AGGAGAAGGCACCA | 31323 | Colorectal |
| 605 | UGCCUUCUCCU | | UGGGAUUUA | | micrornaome |
| hsa-miR- | AAACUACUGAAA | 29282 | AUCUUUGAUUUUCA | 31324 | Colorectal |
| 606 | AUCAAAGAU | | GUAGUUU | | micrornaome |
| hsa-miR- | GUUCAAAUCCAG | 29283 | GUUAUAGAUCUGGA | 31325 | Colorectal |
| 607 | AUCUAUAAC | | UUUGAAC | | micrornaome |
| hsa-miR- | AGGGUGUUUCUC | 29284 | AGAGAUGAGAGAAA | 31326 | Colorectal |
| 609 | UCAUCUCU | | CACCCU | | micrornaome |
| hsa-miR- | GACCUGGACAUG | 29285 | ACUGGGCACAAACA | 31327 | Colorectal |
| 619 | UUUGUGCCCAGU | | UGUCCAGGUC | | micrornaome |
| hsa-miR- | AUGGAGAUAGAU | 29286 | AUUUCUAUAUCUAU | 31328 | Colorectal |
| 620 | AUAGAAAU | | CUCCAU | | micrornaome |
| hsa-miR- | AGCUGUCUGAAA | 29287 | AAGACAUUUUCAGA | 31329 | Colorectal |
| 626 | AUGUCUU | | CAGCU | | micrornaome |
| hsa-miR- | AGACCUGGCCCAG | 29288 | GCUGAGGUCUGGGC | 31330 | Colorectal |
| 631 | ACCUCAGC | | CAGGUCU | | micrornaome |
| hsa-miR- | ACUUGGGCACUG | 29289 | GGACAUUGUUUCAG | 31331 | Colorectal |
| 635 | AAACAAUGUCC | | UGCCCAAGU | | micrornaome |
| hsa-miR- | ACUGGGGGCUUU | 29290 | ACGCAGAGCCCGAA | 31332 | Colorectal |
| 637 | CGGGCUCUGCGU | | AGCCCCCAGU | | micrornaome |
| hsa-miR- | AUCGCUGCGGUU | 29291 | ACAGCGCUCGCAAC | 31333 | Colorectal |
| 639 | GCGAGCGCUGU | | CGCAGCGAU | | micrornaome |
| hsa-miR- | GUCCCUCUCCAAA | 29292 | CAAGACACAUUUGG | 31334 | Colorectal |
| 642a-5p | UGUGUCUUG | | AGAGGGAC | | micrornaome |
| hsa-miR- | ACUUGUAUGCUA | 29293 | CUACCUGAGCUAGC | 31335 | Colorectal |
| 643 | GCUCAGGUAG | | AUACAAGU | | micrornaome |
| hsa-miR- | UUUAGGAUAAGC | 29294 | CAAAAGUCAAGCUU | 31336 | Colorectal |
| 651 | UUGACUUUUG | | AUCCUAAA | | micrornaome |
| hsa-miR- | GUGUUGAAACAA | 29295 | CAGUAGAGAUUGUU | 31337 | Colorectal |
| 653 | UCUCUACUG | | UCAACAC | | micrornaome |
| hsa-miR- | UAUGUCUGCUGA | 29296 | AAGGUGAUGGUCAG | 31338 | Colorectal |
| 654-3p | CCAUCACCUU | | CAGACAUA | | micrornaome |
| hsa-miR- | GGGGCUGGGGCC | 29297 | GCUCGGCCCCGGCC | 31339 | Corneal epithelial |
| 762 | GGGGCCGAGC | | CCAGCCCC | | cells |
| hsa-let-7c | UGAGGUAGUAGG | 29298 | AACCAUACAACCUA | 31340 | Dendritic cells |
| | UUGUAUGGUU | | CUACCUCA | | |
| hsa-miR- | GUGUCUUUUGCU | 29299 | UGACUGCAGAGCAA | 31341 | Dendritic cells and |
| 511 | CUGCAGUCA | | AAGACAC | | macrophages |
| hsa-miR- | CCCCGGGAACGUC | 29300 | GCUCCAGUCUCGAC | 31342 | Embryoid body |
| 1247-3p | GAGACUGGAGC | | GUUCCCGGGG | | cells |
| hsa-miR- | ACCCGUCCCGUUC | 29301 | UCCGGGGACGAACG | 31343 | Embryoid body |
| 1247-5p | GUCCCCGGA | | GGACGGGU | | cells |
| hsa-miR- | AUGGGUGAAUUU | 29302 | AUCCUUCUACAAAU | 31344 | Embryoid body |
| 1262 | GUAGAAGGAU | | UCACCCAU | | cells |
| hsa-miR- | CUGGACUGAGCCG | 29303 | CCAGUAGCACGGCU | 31345 | Embryoid body |
| 1269a | UGCUACUGG | | CAGUCCAG | | cells |
| hsa-miR- | CUGGACUGAGCCA | 29304 | CCAGUAGCAUGGCU | 31346 | Embryoid body |
| 1269b | UGCUACUGG | | CAGUCCAG | | cells |
| hsa-miR- | UACGUAGAUAUA | 29305 | AAAAUACAUAUAUA | 31347 | Embryoid body |
| 1277-3p | UAUGUAUUUU | | UCUACGUA | | cells |
| hsa-miR- | AAAUAUAUAUAU | 29306 | GCUCUUUUCAUUAA | 31348 | Embryoid body |
| 1277-5p | AUAUGUACGUAU | | UGUAGACA | | cells |
| hsa-miR- | UGCUGGAUCAGU | 29307 | GACUCGAACCACUG | 31349 | Embryoid body |
| 1287 | GGUUCGAGUC | | AUCCAGCA | | cells |
| hsa-miR- | UGGAUUUUUGGA | 29308 | UCCCUGAUCCAAAA | 31350 | Embryoid body |
| 1290 | UCAGGGA | | AUCCA | | cells |
| hsa-miR- | UUAUAAAGCAAU | 29309 | AAUCAGUCUCAUUG | 31351 | Embryoid body |
| 340-5p | GAGACUGAUU | | CUUUAUAA | | cells |
| hsa-miR- | UAGUGCAAUAUU | 29310 | ACCCUAUAAGCAAU | 31352 | Embryoid body |
| 454-3p | GCUUAUAGGGU | | AUUGCACUA | | cells, central |
| | | | | | nervous system, |
| | | | | | monocytes |
| hsa-miR- | ACCCUAUCAAUAU | 29311 | GCAGAGACAAUAUU | 31353 | Embryoid body |
| 454-5p | UGUCUCUGC | | GAUAGGGU | | cells, central |
| | | | | | nervous system, |
| | | | | | monocytes |
| hsa-miR- | UAAGUGCUUCCA | 29312 | AAGCAUGGAAGCAC | 31354 | Embryonic body |
| 302e | UGCUU | | UUA | | cells |
| hsa-miR- | UCGGCCUGACCAC | 29313 | GUGGGGUGGGUGGU | 31355 | Embryonic stem |
| 1234-3p | CCACCCCAC | | CAGGCCGA | | cell |
| hsa-miR- | GGGGGGGGGGGG | 29314 | CGGCCCCCCCCCCCC | 31356 | Embryonic stem |
| 1234-5p | GGGGGGCCG | | CCCCCC | | cell |
| hsa-let- | CUAUACGACCUGC | 29315 | AGAAAGGCAGCAGG | 31357 | Embryonic stem |
| 7d-3p | UGCCUUUCU | | UCGUAUAG | | cells |
| hsa-let- | AGAGGUAGUAGG | 29316 | AACUAUGCAACCUA | 31358 | Embryonic stem |
| 7d-5p | UUGCAUAGUU | | CUACCUCU | | cells |
| hsa-miR- | CCGCACUGUGGGU | 29317 | GCAGCAAGUACCCA | 31359 | Embryonic stem |
| 106b-3p | ACUUGCUGC | | CAGUGCGG | | cells |
| hsa-miR- | UAAAGUGCUGAC | 29318 | AUCUGCACUGUCAG | 31360 | Embryonic stem |
| 106b-5p | AGUGCAGAU | | CACUUUA | | cells |
| hsa-miR- | AACUGGAUCAAU | 29319 | CACUCCUAUAAUUG | 31361 | Embryonic stem |
| 1243 | UAUAGGAGUG | | AUCCAGUU | | cells |
| hsa-miR- | AAGUAGUUGGUU | 29320 | AACCAUCUCAUACA | 31362 | Embryonic stem |
| 1244 | UGUAUGAGAUGG | | AACCAACUACUU | | cells |
| | UU | | | | |
| hsa-miR- | AAGUGAUCUAAA | 29321 | AUGUAGGCCUUUAG | 31363 | Embryonic stem |
| 1245a | GGCCUACAU | | AUCACUU | | cells |
| hsa-miR- | UCAGAUGAUCUA | 29322 | UAUAGGCCUUUAGA | 31364 | Embryonic stem |
| 1245b-3p | AAGGCCUAUA | | UCAUCUGA | | cells |
| hsa-miR- | UAGGCCUUUAGA | 29323 | UUUAAGUGAUCUAA | 31365 | Embryonic stem |
| 1245b-5p | UCACUUAAA | | AGGCCUA | | cells |
| hsa-miR- | ACUCUAGCUGCCA | 29324 | AGCGCCUUUGGCAG | 31366 | Embryonic stem |
| 1251 | AAGGCGCU | | CUAGAGU | | cells |
| hsa-miR- | AGAAGGAAAUUG | 29325 | UAAAUGAAUUCAAU | 31367 | Embryonic stem |
| 1252 | AAUUCAUUUA | | UUCCUUCU | | cells |
| hsa-miR- | AGAGAAGAAGAU | 29326 | UGCAGGCUGAUCUU | 31368 | Embryonic stem |
| 1253 | CAGCCUGCA | | CUUCUCU | | cells |
| hsa-miR- | AGCCUGGAAGCU | 29327 | ACUGCAGGCUCCAG | 31369 | Embryonic stem |
| 1254 | GGAGCCUGCAGU | | CUUCCAGGCU | | cells |
| hsa-miR- | AGGAUGAGCAAA | 29328 | AAUCUACUUUCUUU | 31370 | Embryonic stem |
| 1255a | GAAAGUAGAUU | | GCUCAUCCU | | cells |
| hsa-miR- | AACCACUUUCUUU | 29329 | UGGAUGAGCAAAGA | 31371 | Embryonic stem |
| 1255b-2-3p | GCUCAUCCA | | AAGUGGUU | | cells |
| hsa-miR- | CGGAUGAGCAAA | 29330 | AACCACUUUCUUUG | 31372 | Embryonic stem |
| 1255b-5p | GAAAGUGGUU | | CUCAUCCG | | cells |
| hsa-miR- | AGGCAUUGACUU | 29331 | AGCUAGUGAGAAGU | 31373 | Embryonic stem |
| 1256 | CUCACUAGCU | | CAAUGCCU | | cells |
| hsa-miR- | AGUGAAUGAUGG | 29332 | GGUCAGAACCCAUC | 31374 | Embryonic stem |
| 1257 | GUUCUGACC | | AUUCACU | | cells |
| hsa-miR- | AGUUAGGAUUAG | 29333 | UUCCACGACCUAAU | 31375 | Embryonic stem |
| 1258 | GUCGUGGAA | | CCUAACU | | cells |
| hsa-miR- | AUGGAUAAGGCU | 29334 | AAGCCAAAGCCUUA | 31376 | Embryonic stem |
| 1261 | UUGGCUU | | UCCAU | | cells |
| hsa-miR- | AUGGUACCCUGGC | 29335 | ACUCAGUAUGCCAG | 31377 | Embryonic stem |
| 1263 | AUACUGAGU | | GGUACCAU | | cells |
| hsa-miR- | CAAGUCUUAUUU | 29336 | AACAGGUGCUCAAA | 31378 | Embryonic stem |
| 1264 | GAGCACCUGUU | | UAAGACUUG | | cells |
| hsa-miR- | CAGGAUGUGGUC | 29337 | AACAACACUUGACC | 31379 | Embryonic stem |
| 1265 | AAGUGUUGUU | | ACAUCCUG | | cells |
| hsa-miR- | CCUCAGGGCUGUA | 29338 | AGCCCUGUUCUACA | 31380 | Embryonic stem |
| 1266 | GAACAGGGCU | | GCCCUGAGG | | cells |
| hsa-miR- | CCUGUUGAAGUG | 29339 | UGGGGAUUACACUU | 31381 | Embryonic stem |
| 1267 | UAAUCCCCA | | CAACAGG | | cells |
| hsa-miR- | CGGGCGUGGUGG | 29340 | CCCCCACCACCACG | 31382 | Embryonic stem |
| 1268a | UGGGGG | | CCCG | | cells |
| hsa-miR- | CGGGCGUGGUGG | 29341 | CACCCCCACCACCA | 31383 | Embryonic stem |
| 1268b | UGGGGGUG | | CGCCCG | | cells |
| hsa-miR- | CUGGAGAUAUGG | 29342 | ACACAGCUCUUCCA | 31384 | Embryonic stem |
| 1270 | AAGAGCUGUGU | | UAUCUCCAG | | cells |
| hsa-miR- | GAUGAUGAUGGC | 29343 | UUUCAGAAUUUGCU | 31385 | Embryonic stem |
| 1272 | AGCAAAUUCUGA | | GCCAUCAUCAUC | | cells |
| | AA | | | | |
| hsa-miR- | GGGCGACAAAGC | 29344 | AAGAAAGAGUCUUG | 31386 | Embryonic stem |
| 1273a | AAGACUCUUUCU | | CUUUGUCGCCC | | cells |
| | U | | | | |
| hsa-miR- | GAACCCAUGAGG | 29345 | ACUGCAGCCUCAAC | 31387 | Embryonic stem |
| 1273d | UUGAGGCUGCAG | | CUCAUGGGUUC | | cells |
| | U | | | | |
| hsa-miR- | GUGGGGGAGAGG | 29346 | GACAGCCUCUCCCC | 31388 | Embryonic stem |
| 1275 | CUGUC | | CAC | | cells |
| hsa-miR- | UAAAGAGCCCUG | 29347 | UGUCUCCACAGGGC | 31389 | Embryonic stem |
| 1276 | UGGAGACA | | UCUUUA | | cells |
| hsa-miR- | UAGUACUGUGCA | 29348 | AUAGAUGAUAUGCA | 31390 | Embryonic stem |
| 1278 | UAUCAUCUAU | | CAGUACUA | | cells |
| hsa-miR- | UCGUUUGCCUUU | 29349 | AAGCAGAAAAAGGC | 31391 | Embryonic stem |
| 1282 | UUCUGCUU | | AAACGA | | cells |
| hsa-miR- | UGGACUGCCCUGA | 29350 | UCUCCAGAUCAGGG | 31392 | Embryonic stem |
| 1288 | UCUGGAGA | | CAGUCCA | | cells |
| hsa-miR- | UGGGUGGUCUGG | 29351 | GCACAAAUCUCCAG | 31393 | Embryonic stem |
| 1293 | AGAUUUGUGC | | ACCACCCA | | cells |
| hsa-miR- | UGUGAGGUUGGC | 29352 | AGACAACAAUGCCA | 31394 | Embryonic stem |
| 1294 | AUUGUUGUCU | | ACCUCACA | | cells |
| hsa-miR- | UUCAAGUAAUUC | 29353 | CACCUGAAUUACUU | 31395 | Embryonic stem |
| 1297 | AGGUG | | GAA | | cells |
| hsa-miR- | UUCUGGAAUUCU | 29354 | UCCCUCACACAGAA | 31396 | Embryonic stem |
| 1299 | GUGUGAGGGA | | UUCCAGAA | | cells |
| hsa-miR- | UUUUCAACUCUA | 29355 | UCUCUCCCAUUAGA | 31397 | Embryonic stem |
| 1305 | AUGGGAGAGA | | GUUGAAAA | | cells |
| hsa-miR- | ACGUUGGCUCUG | 29356 | CACCACCAGAGCCA | 31398 | Embryonic stem |
| 1306-3p | GUGGUG | | ACGU | | cells |
| hsa-miR- | CCACCUCCCCUGC | 29357 | UGGACGUUUGCAGG | 31399 | Embryonic stem |
| 1306-5p | AAACGUCCA | | GGAGGUGG | | cells |
| hsa-miR- | ACUCGGCGUGGCG | 29358 | CACGACCGACGCCA | 31400 | Embryonic stem |
| 1307-3p | UCGGUCGUG | | CGCCGAGU | | cells |
| hsa-miR- | UCGACCGGACCUC | 29359 | AGCCGGUCGAGGUC | 31401 | Embryonic stem |
| 1307-5p | GACCGGCU | | CGGUCGA | | cells |
| hsa-miR- | UGAGAACUGAAU | 29360 | AGCCUAUGGAAUUC | 31402 | Embryonic stem |
| 146b-5p | UCCAUAGGCU | | AGUUCUCA | | cells |
| hsa-miR- | AAUCAUACACGG | 29361 | AAUAGGUCAACCGU | 31403 | Embryonic stem |
| 154-3p | UUGACCUAUU | | GUAUGAUU | | cells |
| hsa-miR- | UAGGUUAUCCGU | 29362 | CGAAGGCAACACGG | 31404 | Embryonic stem |
| 154-5p | GUUGCCUUCG | | AUAACCUA | | cells |
| hsa-miR- | CCAGUCCUGUGCC | 29363 | AGGCGGCAGGCACA | 31405 | Embryonic stem |
| 1910 | UGCCGCCU | | GGACUGG | | cells |
| hsa-miR- | UCUGCCCCCUCCG | 29364 | UGGCAGCAGCGGAG | 31406 | Embryonic stem |
| 1913 | CUGCUGCCA | | GGGGCAGA | | cells |
| hsa-miR- | GGAGGGGUCCCGC | 29365 | CCUCCCAGUGCGGG | 31407 | Embryonic stem |
| 1914-3p | ACUGGGAGG | | ACCCCUCC | | cells |
| hsa-miR- | CCCUGUGCCCGGC | 29366 | CAGAAGUGGGCCGG | 31408 | Embryonic stem |
| 1914-5p | CCACUUCUG | | GCACAGGG | | cells |
| hsa-miR- | CCCCAGGGCGACG | 29367 | CCCGCCGCGUCGCC | 31409 | Embryonic stem |
| 1915-3p | CGGCGGG | | CUGGGG | | cells |
| hsa-miR- | ACCUUGCCUUGCU | 29368 | GGCCCGGGCAGCAA | 31410 | Embryonic stem |
| 1915-5p | GCCCGGGCC | | GGCAAGGU | | cells |
| hsa-miR- | AUUUGUGCUUGG | 29369 | GUGACAGAGCCAAG | 31411 | Embryonic stem |
| 2113 | CUCUGUCAC | | CACAAAU | | cells |
| hsa-miR- | AUUGUCCUUGCU | 29370 | AUCUCCAAACAGCA | 31412 | Embryonic stem |
| 2355-3p | GUUUGGAGAU | | AGGACAAU | | cells |
| hsa-miR- | AUCCCCAGAUACA | 29371 | UUGUCCAUUGUAUC | 31413 | Embryonic stem |
| 2355-5p | AUGGACAA | | UGGGGAU | | cells |
| hsa-miR- | CAGUGCAAUAGU | 29372 | GCUUUGACAAUACU | 31414 | Embryonic stem |
| 301a-3p | AUUGUCAAAGC | | AUUGCACUG | | cells |
| hsa-miR- | GCUCUGACUUUA | 29373 | AGUAGUGCAAUAAA | 31415 | Embryonic stem |
| 30la-5p | UUGCACUACU | | GUCAGAGC | | cells |
| hsa-miR- | UAAGUGCUUCCA | 29374 | CUACUAAAACAUGG | 31416 | Embryonic stem |
| 3026-3p | UGUUUUAGUAG | | AAGCACUUA | | cells |
| hsa-miR- | ACUUUAACAUGG | 29375 | GAAAGCACUUCCAU | 31417 | Embryonic stem |
| 302b-5p | AAGUGCUUUC | | GUUAAAGU | | cells |
| hsa-miR- | UAAGUGCUUCCA | 29376 | CCACUGAAACAUGG | 31418 | Embryonic stem |
| 302c-3p | UGUUUCAGUGG | | AAGCACUUA | | cells |
| hsa-miR- | UUUAACAUGGGG | 29377 | CAGCAGGUACCCCC | 31419 | Embryonic stem |
| 302c-5p | GUACCUGCUG | | AUGUUAAA | | cells |
| hsa-miR- | UAAGUGCUUCCA | 29378 | ACACUCAAACAUGG | 31420 | Embryonic stem |
| 302d-3p | UGUUUGAGUGU | | AAGCACUUA | | cells |
| hsa-miR- | ACUUUAACAUGG | 29379 | GCAAGUGCCUCCAU | 31421 | Embryonic stem |
| 302d-5p | AGGCACUUGC | | GUUAAAGU | | cells |
| hsa-miR- | AAUUGCACUUUA | 29380 | UCACCAUUGCUAAA | 31422 | Embryonic stem |
| 367-3p | GCAAUGGUGA | | GUGCAAUU | | cells |
| hsa-miR- | ACUGUUGCUAAU | 29381 | AGAGUUGCAUAUUA | 31423 | Embryonic stem |
| 367-5p | AUGCAACUCU | | GCAACAGU | | cells |
| hsa-miR- | AGCUCGGUCUGA | 29382 | ACUGAGGGGCCUCA | 31424 | Embryonic stem |
| 423-3p | GGCCCCUCAGU | | GACCGAGCU | | cells |
| hsa-miR- | AAAAACUGAGAC | 29383 | UGCAAAAGUAGUCU | 31425 | Embryonic stem |
| 548e | UACUUUUGCA | | CAGUUUUU | | cells |
| hsa-miR- | AAAAACUGUAAU | 29384 | AAAAGUAAUUACAG | 31426 | Embryonic stem |
| 548f | UACUUUU | | UUUUU | | cells |
| hsa-miR- | AAAACUGUAAUU | 29385 | GUACAAAAGUAAUU | 31427 | Embryonic stem |
| 548g-3p | ACUUUUGUAC | | ACAGUUUU | | cells |
| hsa-miR- | UGCAAAAGUAAU | 29386 | CAAAAACUGCAAUU | 31428 | Embryonic stem |
| 548g-5p | UGCAGUUUUUG | | ACUUUUGCA | | cells |
| hsa-miR- | CAAAAACCGCAAU | 29387 | UGCAAAAGUAAUUG | 31429 | Embryonic stem |
| 548h-3p | UACUUUUGCA | | CGGUUUUUG | | cells |
| hsa-miR- | AAAAGUAAUCGC | 29388 | GACAAAAACCGCGA | 31430 | Embryonic stem |
| 548h-5p | GGUUUUUGUC | | UUACUUUU | | cells |
| hsa-miR- | AAAAGUACUUGC | 29389 | AGCAAAAUCCGCAA | 31431 | Embryonic stem |
| 548k | GGAUUUUGCU | | GUACUUUU | | cells |
| hsa-miR- | AAAAGUAUUUGC | 29390 | GACAAAACCCGCAA | 31432 | Embryonic stem |
| 5481 | GGGUUUUGUC | | AUACUUUU | | cells |
| hsa-miR- | CAAAGGUAUUUG | 29391 | CAAAAACCACAAAU | 31433 | Embryonic stem |
| 548m | UGGUUUUUG | | ACCUUUG | | cells |
| hsa-miR- | CCAAAACUGCAGU | 29392 | GCAAAAGUAACUGC | 31434 | Embryonic stem |
| 5480-3p | UACUUUUGC | | AGUUUUGG | | cells |
| hsa-miR- | AAAAGUAAUUGC | 29393 | GGCAAAAACCGCAA | 31435 | Embryonic stem |
| 5480-5p | GGUUUUUGCC | | UUACUUUU | | cells |
| hsa-miR- | UAGCAAAAACUG | 29394 | AAAGUAACUGCAGU | 31436 | Embryonic stem |
| 548p | CAGUUACUUU | | UUUUGCUA | | cells |
| hsa-miR- | GGAGGUUGGGAA | 29395 | CUCUGCCCUUCCCA | 31437 | Embryonic stem |
| 6086 | GGGCAGAG | | ACCUCC | | cells |
| hsa-miR- | UGAGGCGGGGGG | 29396 | GCUCGCCCCCCCGC | 31438 | Embryonic stem |
| 6087 | GCGAGC | | CUCA | | cells |
| hsa-miR- | AGAGAUGAAGCG | 29397 | CGCCCCCCCGCUUC | 31439 | Embryonic stem |
| 6088 | GGGGGGCG | | AUCUCU | | cells |
| hsa-miR- | GGAGGCCGGGGU | 29398 | CCGCCCCGCCCCACC | 31440 | Embryonic stem |
| 6089 | GGGGGGGGGGGG | | CCGGCCUCC | | cells |
| hsa-miR- | GGGGAGCGAGGG | 29399 | GCCCCGCCCCUCGC | 31441 | Embryonic stem |
| 6090 | GCGGGGC | | UCCCC | | cells |
| hsa-miR- | UAUUCAUUUAUC | 29400 | UGUAGGCUGGGGAU | 31442 | Embryonic stem |
| 664a-3p | CCCAGCCUACA | | AAAUGAAUA | | cells |
| hsa-miR- | ACUGGCUAGGGA | 29401 | AUCCAAUCAUUUUC | 31443 | Embryonic stem |
| 664a-5p | AAAUGAUUGGAU | | CCUAGCCAGU | | cells |
| hsa-miR- | UUCAUUUGCCUCC | 29402 | UGUAGGCUGGGAGG | 31444 | Embryonic stem |
| 664b-3p | CAGCCUACA | | CAAAUGAA | | cells |
| hsa-miR- | UGGGCUAAGGGA | 29403 | UACCCAAUCAUCUC | 31445 | Embryonic stem |
| 6646-5p | GAUGAUUGGGUA | | CCUUAGCCCA | | cells |
| hsa-miR- | ACUCCAGCCCCAC | 29404 | GCUGAGGCUGUGGG | 31446 | Embryonic stem |
| 766-3p | AGCCUCAGC | | GCUGGAGU | | cells |
| hsa-miR- | AGGAGGAAUUGG | 29405 | AAGACCAGCACCAA | 31447 | Embryonic stem |
| 766-5p | UGCUGGUCUU | | UUCCUCCU | | cells |
| hsa-miR- | AGGCAGCGGGGU | 29406 | UAUCCACUACACCC | 31448 | Embryonic stem |
| 885-3p | GUAGUGGAUA | | CGCUGCCU | | cells |
| hsa-miR- | UCCAUUACACUAC | 29407 | AGAGGCAGGGUAGU | 31449 | Embryonic stem |
| 885-5p | CCUGCCUCU | | GUAAUGGA | | cells |
| hsa-miR- | ACUGCUGAGCUA | 29408 | CGGGAAGUGCUAGC | 31450 | Embryonic stem |
| 93-3p | GCACUUCCCG | | UCAGCAGU | | cells |
| hsa-miR- | CAAAGUGCUGUU | 29409 | CUACCUGCACGAAC | 31451 | Embryonic stem |
| 93-5p | CGUGCAGGUAG | | AGCACUUUG | | cells |
| hsa-miR- | CACCCGGCUGUGU | 29410 | GCACAUGUGCACAC | 31452 | Embryonic stem |
| 941 | GCACAUGUGC | | AGCCGGGUG | | cells |
| hsa-miR- | AGCUUCUUUACA | 29411 | CAAGGCAGCACUGU | 31453 | Embryonic stem |
| 103a-2-5p | GUGCUGCCUUG | | AAAGAAGCU | | cells and a variety |
| | | | | | of cells and tissues |
| hsa-miR- | AGCAGCAUUGUA | 29412 | UCAUAGCCCUGUAC | 31454 | Embryonic stem |
| 103a-3p | CAGGGCUAUGA | | AAUGCUGCU | | cells and a variety |
| | | | | | of cells and tissues |
| hsa-miR- | CCUGAGAAAAGG | 29413 | UUGGCCCUUUUCUC | 31455 | Embryonic stem |
| 4251 | GCCAA | | AGG | | cells and neural |
| | | | | | precursors |
| hsa-miR- | GGCCACUGAGUCA | 29414 | UGGUGCUGACUCAG | 31456 | Embryonic stem |
| 4252 | GCACCA | | UGGCC | | cells and neural |
| | | | | | precursors |
| hsa-miR- | AGGGCAUGUCCA | 29415 | ACCCCCUGGACAUG | 31457 | Embryonic stem |
| 4253 | GGGGGU | | CCCU | | cells and neural |
| | | | | | precursors |
| hsa-miR- | GCCUGGAGCUACU | 29416 | GAGAUGGUGGAGUA | 31458 | Embryonic stem |
| 4254 | CCACCAUCUC | | GCUCCAGGC | | cells and neural |
| | | | | | precursors |
| hsa-miR- | CAGUGUUCAGAG | 29417 | UCCAUCUCUGAACA | 31459 | Embryonic stem |
| 4255 | AUGGA | | CUG | | cells and neural |
| | | | | | precursors |
| hsa-miR- | AUCUGACCUGAU | 29418 | ACCUUCAUCAGGUC | 31460 | Embryonic stem |
| 4256 | GAAGGU | | AGAU | | cells and neural |
| | | | | | precursors |
| hsa-miR- | CCAGAGGUGGGG | 29419 | CUCAGUCCCCACCU | 31461 | Embryonic stem |
| 4257 | ACUGAG | | CUGG | | cells and neural |
| | | | | | precursors |
| hsa-miR- | CCCCGCCACCGCC | 29420 | CCAAGGCGGUGGCG | 31462 | Embryonic stem |
| 4258 | UUGG | | GGG | | cells and neural |
| | | | | | precursors |
| hsa-miR- | CAGUUGGGUCUA | 29421 | UCCUGACCCCUAGA | 31463 | Embryonic stem |
| 4259 | GGGGUCAGGA | | CCCAACUG | | cells and neural |
| | | | | | precursors |
| hsa-miR- | CUUGGGGCAUGG | 29422 | UGGGACUCCAUGCC | 31464 | Embryonic stem |
| 4260 | AGUCCCA | | CCAAG | | cells and neural |
| | | | | | precursors |
| hsa-miR- | AGGAAACAGGGA | 29423 | UGGGUCCCUGUUUC | 31465 | Embryonic stem |
| 4261 | CCCA | | CU | | cells and neural |
| | | | | | precursors |
| hsa-miR- | GACAUUCAGACU | 29424 | CAGGUAGUCUGAAU | 31466 | Embryonic stem |
| 4262 | ACCUG | | GUC | | cells and neural |
| | | | | | precursors |
| hsa-miR- | AUUCUAAGUGCC | 29425 | GGCCAAGGCACUUA | 31467 | Embryonic stem |
| 4263 | UUGGCC | | GAAU | | cells and neural |
| | | | | | precursors |
| hsa-miR- | ACUCAGUCAUGG | 29426 | AAUGACCAUGACUG | 31468 | Embryonic stem |
| 4264 | UCAUU | | AGU | | cells and neural |
| | | | | | precursors |
| hsa-miR- | CUGUGGGCUCAGC | 29427 | CCCAGAGCUGAGCC | 31469 | Embryonic stem |
| 4265 | UCUGGG | | CACAG | | cells and neural |
| | | | | | precursors |
| hsa-miR- | CUAGGAGGCCUU | 29428 | GGCCAAGGCCUCCU | 31470 | Embryonic stem |
| 4266 | GGCC | | AG | | cells and neural |
| | | | | | precursors |
| hsa-miR- | UCCAGCUCGGUGG | 29429 | GUGCCACCGAGCUG | 31471 | Embryonic stem |
| 4267 | CAC | | GA | | cells and neural |
| | | | | | precursors |
| hsa-miR- | GGCUCCUCCUCUC | 29430 | CACAUCCUGAGAGG | 31472 | Embryonic stem |
| 4268 | AGGAUGUG | | AGGAGCC | | cells and neural |
| | | | | | precursors |
| hsa-miR- | GCAGGCACAGACA | 29431 | GCCAGGGCUGUCUG | 31473 | Embryonic stem |
| 4269 | GCCCUGGC | | UGCCUGC | | cells and neural |
| | | | | | precursors |
| hsa-miR- | UCAGGGAGUCAG | 29432 | GCCCUCCCCUGACU | 31474 | Embryonic stem |
| 4270 | GGGAGGGC | | CCCUGA | | cells and neural |
| | | | | | precursors |
| hsa-miR- | GGGGGAAGAAAA | 29433 | CCCCACCUUUUCUU | 31475 | Embryonic stem |
| 4271 | GGUGGGG | | CCCCC | | cells and neural |
| | | | | | precursors |
| hsa-miR- | CAUUCAACUAGU | 29434 | ACAAUCACUAGUUG | 31476 | Embryonic stem |
| 4272 | GAUUGU | | AAUG | | cells and neural |
| | | | | | precursors |
| hsa-miR- | CAGCAGUCCCUCC | 29435 | CAGGGGGAGGGACU | 31477 | Embryonic stem |
| 4274 | CCCUG | | GCUG | | cells and neural |
| | | | | | precursors |
| hsa-miR- | CCAAUUACCACUU | 29436 | AAAGAAGUGGUAAU | 31478 | Embryonic stem |
| 4275 | CUUU | | UGG | | cells and neural |
| | | | | | precursors |
| hsa-miR- | CUCAGUGACUCAU | 29437 | GCACAUGAGUCACU | 31479 | Embryonic stem |
| 4276 | GUGC | | GAG | | cells and neural |
| | | | | | precursors |
| hsa-miR- | GCAGUUCUGAGC | 29438 | GUGUACUGUGCUCA | 31480 | Embryonic stem |
| 4277 | ACAGUACAC | | GAACUGC | | cells and neural |
| | | | | | precursors |
| hsa-miR- | CUAGGGGGUUUG | 29439 | CAAGGGCAAACCCC | 31481 | Embryonic stem |
| 4278 | CCCUUG | | CUAG | | cells and neural |
| | | | | | precursors |
| hsa-miR- | CUCUCCUCCCGGC | 29440 | GAAGCCGGGAGGAG | 31482 | Embryonic stem |
| 4279 | UUC | | AG | | cells and neural |
| | | | | | precursors |
| hsa-miR- | GAGUGUAGUUCU | 29441 | GCUCUGCUCAGAAC | 31483 | Embryonic stem |
| 4280 | GAGCAGAGC | | UACACUC | | cells and neural |
| | | | | | precursors |
| hsa-miR- | GGGUCCCGGGGA | 29442 | CCCCCCUCCCCGGG | 31484 | Embryonic stem |
| 4281 | GGGGGG | | ACCC | | cells and neural |
| | | | | | precursors |
| hsa-miR- | UAAAAUUUGCAU | 29443 | UCCUGGAUGCAAAU | 31485 | Embryonic stem |
| 4282 | CCAGGA | | UUUA | | cells and neural |
| | | | | | precursors |
| hsa-miR- | UGGGGCUCAGCG | 29444 | AAACUCGCUGAGCC | 31486 | Embryonic stem |
| 4283 | AGUUU | | CCA | | cells and neural |
| | | | | | precursors |
| hsa-miR- | GGGCUCACAUCAC | 29445 | AUGGGGUGAUGUGA | 31487 | Embryonic stem |
| 4284 | CCCAU | | GCCC | | cells and neural |
| | | | | | precursors |
| hsa-miR- | GCGGCGAGUCCGA | 29446 | AUGAGUCGGACUCG | 31488 | Embryonic stem |
| 4285 | CUCAU | | CCGC | | cells and neural |
| | | | | | precursors |
| hsa-miR- | ACCCCACUCCUGG | 29447 | GGUACCAGGAGUGG | 31489 | Embryonic stem |
| 4286 | UACC | | GGU | | cells and neural |
| | | | | | precursors |
| hsa-miR- | UCUCCCUUGAGGG | 29448 | AAAGUGCCCUCAAG | 31490 | Embryonic stem |
| 4287 | CACUUU | | GGAGA | | cells and neural |
| | | | | | precursors |
| hsa-miR- | UUGUCUGCUGAG | 29449 | GGAAACUCAGCAGA | 31491 | Embryonic stem |
| 4288 | UUUCC | | CAA | | cells and neural |
| | | | | | precursors |
| hsa-miR- | GCAUUGUGCAGG | 29450 | UGAUAGCCCUGCAC | 31492 | Embryonic stem |
| 4289 | GCUAUCA | | AAUGC | | cells and neural |
| | | | | | precursors |
| hsa-miR- | UGCCCUCCUUUCU | 29451 | GAGGGAAGAAAGGA | 31493 | Embryonic stem |
| 4290 | UCCCUC | | GGGCA | | cells and neural |
| | | | | | precursors |
| hsa-miR- | UUCAGCAGGAAC | 29452 | AGCUGUUCCUGCUG | 31494 | Embryonic stem |
| 4291 | AGCU | | AA | | cells and neural |
| | | | | | precursors |
| hsa-miR- | CCCCUGGGCCGGC | 29453 | CCAAGGCCGGCCCA | 31495 | Embryonic stem |
| 4292 | CUUGG | | GGGG | | cells and neural |
| | | | | | precursors |
| hsa-miR- | CAGCCUGACAGGA | 29454 | CUGUUCCUGUCAGG | 31496 | Embryonic stem |
| 4293 | ACAG | | CUG | | cells and neural |
| | | | | | precursors |
| hsa-miR- | GGGAGUCUACAG | 29455 | CCCUGCUGUAGACU | 31497 | Embryonic stem |
| 4294 | CAGGG | | CCC | | cells and neural |
| | | | | | precursors |
| hsa-miR- | CAGUGCAAUGUU | 29456 | AAGGAAAACAUUGC | 31498 | Embryonic stem |
| 4295 | UUCCUU | | ACUG | | cells and neural |
| | | | | | precursors |
| hsa-miR- | AUGUGGGCUCAG | 29457 | UGAGCCUGAGCCCA | 31499 | Embryonic stem |
| 4296 | GCUCA | | CAU | | cells and neural |
| | | | | | precursors |
| hsa-miR- | UGCCUUCCUGUCU | 29458 | CACAGACAGGAAGG | 31500 | Embryonic stem |
| 4297 | GUG | | CA | | cells and neural |
| | | | | | precursors |
| hsa-miR- | CUGGGACAGGAG | 29459 | CUGCCUCCUCCUCC | 31501 | Embryonic stem |
| 4298 | GAGGAGGCAG | | UGUCCCAG | | cells and neural |
| | | | | | precursors |
| hsa-miR- | GCUGGUGACAUG | 29460 | GCCUCUCAUGUCAC | 31502 | Embryonic stem |
| 4299 | AGAGGC | | CAGC | | cells and neural |
| | | | | | precursors |
| hsa-miR- | UGGGAGCUGGAC | 29461 | GAAGUAGUCCAGCU | 31503 | Embryonic stem |
| 4300 | UACUUC | | CCCA | | cells and neural |
| | | | | | precursors |
| hsa-miR- | UCCCACUACUUCA | 29462 | UCACAAGUGAAGUA | 31504 | Embryonic stem |
| 4301 | CUUGUGA | | GUGGGA | | cells and neural |
| | | | | | precursors |
| hsa-miR- | CCAGUGUGGCUCA | 29463 | CUCGCUGAGCCACA | 31505 | Embryonic stem |
| 4302 | GCGAG | | CUGG | | cells and neural |
| | | | | | precursors |
| hsa-miR- | UUCUGAGCUGAG | 29464 | CUGUCCUCAGCUCA | 31506 | Embryonic stem |
| 4303 | GACAG | | GAA | | cells and neural |
| | | | | | precursors |
| hsa-miR- | CCGGCAUGUCCAG | 29465 | UGCCCUGGACAUGC | 31507 | Embryonic stem |
| 4304 | GGCA | | CGG | | cells and neural |
| | | | | | precursors |
| hsa-miR- | CCUAGACACCUCC | 29466 | GAACUGGAGGUGUC | 31508 | Embryonic stem |
| 4305 | AGUUC | | UAGG | | cells and neural |
| | | | | | precursors |
| hsa-miR- | UGGAGAGAAAGG | 29467 | UACUGCCUUUCUCU | 31509 | Embryonic stem |
| 4306 | CAGUA | | CCA | | cells and neural |
| | | | | | precursors |
| hsa-miR- | AAUGUUUUUUCC | 29468 | GGAAACAGGAAAAA | 31510 | Embryonic stem |
| 4307 | UGUUUCC | | ACAUU | | cells and neural |
| | | | | | precursors |
| hsa-miR- | UCCCUGGAGUUUC | 29469 | AAGAAGAAACUCCA | 31511 | Embryonic stem |
| 4308 | UUCUU | | GGGA | | cells and neural |
| | | | | | precursors |
| hsa-miR- | CUGGAGUCUAGG | 29470 | UGGAAUCCUAGACU | 31512 | Embryonic stem |
| 4309 | AUUCCA | | CCAG | | cells and neural |
| | | | | | precursors |
| hsa-miR- | GCAGCAUUCAUG | 29471 | GGGACAUGAAUGCU | 31513 | Embryonic stem |
| 4310 | UCCC | | GC | | cells and neural |
| | | | | | precursors |
| hsa-miR- | GAAAGAGAGCUG | 29472 | CACACUCAGCUCUC | 31514 | Embryonic stem |
| 4311 | AGUGUG | | UUUC | | cells and neural |
| | | | | | precursors |
| hsa-miR- | GGCCUUGUUCCUG | 29473 | UGGGGACAGGAACA | 31515 | Embryonic stem |
| 4312 | UCCCCA | | AGGCC | | cells and neural |
| | | | | | precursors |
| hsa-miR- | AGCCCCCUGGCCC | 29474 | GGGUUUGGGGCCAG | 31516 | Embryonic stem |
| 4313 | CAAACCC | | GGGGCU | | cells and neural |
| | | | | | precursors |
| hsa-miR- | CUCUGGGAAAUG | 29475 | CUGUCCCAUUUCCC | 31517 | Embryonic stem |
| 4314 | GGACAG | | AGAG | | cells and neural |
| | | | | | precursors |
| hsa-miR- | CCGCUUUCUGAGC | 29476 | GUCCAGCUCAGAAA | 31518 | Embryonic stem |
| 4315 | UGGAC | | GCGG | | cells and neural |
| | | | | | precursors |
| hsa-miR- | GGUGAGGCUAGC | 29477 | CACCAGCUAGCCUC | 31519 | Embryonic stem |
| 4316 | UGGUG | | ACC | | cells and neural |
| | | | | | precursors |
| hsa-miR- | ACAUUGCCAGGG | 29478 | AAACUCCCUGGCAA | 31520 | Embryonic stem |
| 4317 | AGUUU | | UGU | | cells and neural |
| | | | | | precursors |
| hsa-miR- | CACUGUGGGUAC | 29479 | AGCAUGUACCCACA | 31521 | Embryonic stem |
| 4318 | AUGCU | | GUG | | cells and neural |
| | | | | | precursors |
| hsa-miR- | UCCCUGAGCAAAG | 29480 | GUGGCUUUGCUCAG | 31522 | Embryonic stem |
| 4319 | CCAC | | GGA | | cells and neural |
| | | | | | precursors |
| hsa-miR- | GGGAUUCUGUAG | 29481 | AGGAAGCUACAGAA | 31523 | Embryonic stem |
| 4320 | CUUCCU | | UCCC | | cells and neural |
| | | | | | precursors |
| hsa-miR- | UUAGCGGUGGAC | 29482 | CGCAGGGCGGUCCA | 31524 | Embryonic stem |
| 4321 | CGCCCUGCG | | CCGCUAA | | cells and neural |
| | | | | | precursors |
| hsa-miR- | CUGUGGGCUCAGC | 29483 | CCCCACGCGCUGAG | 31525 | Embryonic stem |
| 4322 | GCGUGGGG | | CCCACAG | | cells and neural |
| | | | | | precursors |
| hsa-miR- | CAGCCCCACAGCC | 29484 | UCUGAGGCUGUGGG | 31526 | Embryonic stem |
| 4323 | UCAGA | | GCUG | | cells and neural |
| | | | | | precursors |
| hsa-miR- | CCCUGAGACCCUA | 29485 | UUAAGGUUAGGGUC | 31527 | Embryonic stem |
| 4324 | ACCUUAA | | UCAGGG | | cells and neural |
| | | | | | precursors |
| hsa-miR- | UUGCACUUGUCUC | 29486 | UCACUGAGACAAGU | 31528 | Embryonic stem |
| 4325 | AGUGA | | GCAA | | cells and neural |
| | | | | | precursors |
| hsa-miR- | UGUUCCUCUGUCU | 29487 | GUCUGGGAGACAGA | 31529 | Embryonic stem |
| 4326 | CCCAGAC | | GGAACA | | cells and neural |
| | | | | | precursors |
| hsa-miR- | GGCUUGCAUGGG | 29488 | CCAGUCCCCCAUGC | 31530 | Embryonic stem |
| 4327 | GGACUGG | | AAGCC | | cells and neural |
| | | | | | precursors |
| hsa-miR- | CCAGUUUUCCCAG | 29489 | AAUCCUGGGAAAAC | 31531 | Embryonic stem |
| 4328 | GAUU | | UGG | | cells and neural |
| | | | | | precursors |
| hsa-miR- | CCUGAGACCCUAG | 29490 | GUGGAACUAGGGUC | 31532 | Embryonic stem |
| 4329 | UUCCAC | | UCAGG | | cells and neural |
| | | | | | precursors |
| hsa-miR- | CCUCAGAUCAGAG | 29491 | GCAAGGCUCUGAUC | 31533 | Embryonic stem |
| 4330 | CCUUGC | | UGAGG | | cells and neural |
| | | | | | precursors |
| hsa-miR- | CAUUGCACUUGUC | 29492 | UCAGACCGAGACAA | 31534 | Embryonic stem |
| 25-3p | UCGGUCUGA | | GUGCAAUG | | cells, airway |
| | | | | | smooth muscle |
| hsa-miR- | AGGCGGAGACUU | 29493 | CAAUUGCCCAAGUC | 31535 | Embryonic stem |
| 25-5p | GGGCAAUUG | | UCCGCCU | | cells, airway |
| | | | | | smooth muscle |
| hsa-miR- | CCUAUUCUUGGU | 29494 | CGUGCAAGUAACCA | 31536 | Embryonic stem |
| 26a-1-3p | UACUUGCACG | | AGAAUAGG | | cells, Blood and |
| | | | | | other tissues |
| hsa-miR- | AAUGGAUUUUUG | 29495 | CCUGCUCCAAAAAU | 31537 | Embryonic stem |
| 1246 | GAGCAGG | | CCAUU | | cells, epithelial cells |
| hsa-miR- | UGCGGGGCUAGG | 29496 | UGCUGUUAGCCCUA | 31538 | Embryonic stem |
| 744-5p | GCUAACAGCA | | GCCCCGCA | | cells, heart |
| hsa-miR- | AAAAGUAAUUGC | 29497 | GGCAAAAUCCGCAA | 31539 | Embryonic stem |
| 548i | GGAUUUUGCC | | UUACUUUU | | cells, immune cells |
| hsa-miR- | CAAAAGUAAUUG | 29498 | ACAAAAUCCACAAU | 31540 | Embryonic stem |
| 548n | UGGAUUUUGU | | UACUUUUG | | cells, immune cells |
| hsa-miR- | UAAGUGCUUCCA | 29499 | UCACCAAAACAUGG | 31541 | Embryonic stem |
| 302a-3p | UGUUUUGGUGA | | AAGCACUUA | | cells, lipid |
| | | | | | metabolism |
| hsa-miR- | ACUUAAACGUGG | 29500 | AGCAAGUACAUCCA | 31542 | Embryonic stem |
| 302a-5p | AUGUACUUGCU | | CGUUUAAGU | | cells, lipid |
| | | | | | metabolism |
| hsa-let- | CUAUACAAUCUAC | 29501 | GAAAGACAGUAGAU | 31543 | Embryonic stem |
| 7a-3p | UGUCUUUC | | UGUAUAG | | cells, lung |
| hsa-let- | UGAGGUAGUAGG | 29502 | AACUAUACAACCUA | 31544 | Embryonic stem |
| 7a-5p | UUGUAUAGUU | | CUACCUCA | | cells, lung |
| hsa-let- | CUGUACAGCCUCC | 29503 | GGAAAGCUAGGAGG | 31545 | Embryonic stem |
| 7a-2-3p | UAGCUUUCC | | CUGUACAG | | cells, lung, myeloid |
| | | | | | cells |
| hsa-miR- | CACCAGGCAUUGU | 29504 | GGAGACCACAAUGC | 31546 | Embryonic stem |
| 1911-3p | GGUCUCC | | CUGGUG | | cells, neural |
| | | | | | precursor |
| hsa-miR- | UGAGUACCGCCAU | 29505 | CCCAACAGACAUGG | 31547 | Embryonic stem |
| 1911-5p | GUCUGUUGGG | | CGGUACUCA | | cells, neural |
| | | | | | precursor |
| hsa-miR- | UACCCAGAGCAUG | 29506 | UUCACACUGCAUGC | 31548 | Embryonic stem |
| 1912 | CAGUGUGAA | | UCUGGGUA | | cells, neural |
| | | | | | precursor |
| hsa-miR- | AAGUGCUGUCAU | 29507 | GACCUCAGCUAUGA | 31549 | Embryonic stem |
| 512-3p | AGCUGAGGUC | | CAGCACUU | | cells, placenta |
| hsa-miR- | CACUCAGCCUUGA | 29508 | GAAAGUGCCCUCAA | 31550 | Embryonic stem |
| 512-5p | GGGCACUUUC | | GGCUGAGUG | | cells, placenta, |
| hsa-miR- | UCGACAGCACGAC | 29509 | GAAGGCAGUGUCGU | 31551 | Endometrial tissues |
| 196b-3p | ACUGCCUUC | | GCUGUCGA | | |
| hsa-miR- | UAGGUAGUUUCC | 29510 | CCCAACAACAGGAA | 31552 | Endometrial tissues |
| 196b-5p | UGUUGUUGGG | | ACUACCUA | | |
| hsa-miR- | UACAGUACUGUG | 29511 | UUCAGUUAUCACAG | 31553 | Endothelial cells |
| 101-3p | AUAACUGAA | | UACUGUA | | |
| hsa-miR- | CAGUUAUCACAG | 29512 | AGCAUCAGCACUGU | 31554 | Endothelial cells |
| 101-5p | UGCUGAUGCU | | GAUAACUG | | |
| hsa-miR- | UGUGCAAAUCUA | 29513 | UCAGUUUUGCAUAG | 31555 | Endothelial cells |
| 19a-3p | UGCAAAACUGA | | AUUUGCACA | | |
| hsa-miR- | AGUUUUGCAUAG | 29514 | UGUAGUGCAACUAU | 31556 | Endothelial cells |
| 19a-5p | UUGCACUACA | | GCAAAACU | | |
| hsa-miR- | AGUUUUGCAGGU | 29515 | GCUGGAUGCAAACC | 31557 | Endothelial cells |
| 19b-1-5p | UUGCAUCCAGC | | UGCAAAACU | | |
| hsa-miR- | AGUUUUGCAGGU | 29516 | UGAAAUGCAAACCU | 31558 | Endothelial cells |
| 19b-2-5p | UUGCAUUUCA | | GCAAAACU | | |
| hsa-miR- | UGUGCAAAUCCA | 29517 | UCAGUUUUGCAUGG | 31559 | Endothelial cells |
| 19b-3p | UGCAAAACUGA | | AUUUGCACA | | |
| hsa-miR- | UACUGCAUCAGG | 29518 | UCCAAUCAGUUCCU | 31560 | Endothelial cells |
| 217 | AACUGAUUGGA | | GAUGCAGUA | | |
| hsa-miR- | AUGGUUCCGUCA | 29519 | CCAUGGUGCUUGAC | 31561 | Endothelial cells |
| 218-1-3p | AGCACCAUGG | | GGAACCAU | | |
| hsa-miR- | AGCUACAUCUGGC | 29520 | ACCCAGUAGCCAGA | 31562 | Endothelial cells |
| 222-3p | UACUGGGU | | UGUAGCU | | |
| hsa-miR- | CUCAGUAGCCAGU | 29521 | AGGAUCUACACUGG | 31563 | Endothelial cells |
| 222-5p | GUAGAUCCU | | CUACUGAG | | |
| hsa-miR- | UUAUCAGAAUCU | 29522 | GUACCCCUGGAGAU | 31564 | Endothelial cells |
| 361-5p | CCAGGGGUAC | | UCUGAUAA | | |
| hsa-miR- | AUCAACAGACAU | 29523 | GCGCCCAAUUAAUG | 31565 | Endothelial cells |
| 421 | UAAUUGGGCGC | | UCUGUUGAU | | |
| hsa-miR- | CAAAACGUGAGG | 29524 | AUAGCAGCGCCUCA | 31566 | Endothelial cells |
| 424-3p | CGCUGCUAU | | CGUUUUG | | |
| hsa-miR- | CAGCAGCAAUUCA | 29525 | UUCAAAACAUGAAU | 31567 | Endothelial cells |
| 424-5p | UGUUUUGAA | | UGCUGCUG | | |
| hsa-miR- | UUCACAGGGAGG | 29526 | AUGACACCUCCCUG | 31568 | Endothelial cells |
| 513a-5p | UGUCAU | | UGAA | | |
| hsa-miR- | GCGGAGAGAGAA | 29527 | GCUCCCCAUUCUCU | 31569 | Endothelial cells |
| 5739 | UGGGGAGC | | CUCCGC | | |
| hsa-miR- | CCUGCGAGUCUCC | 29528 | CCACCGCCGGAGAC | 31570 | Endothelial cells |
| 6068 | GGCGGUGG | | UCGCAGG | | |
| hsa-miR- | GGGCUAGGGCCU | 29529 | GGGGGCAGCAGGCC | 31571 | Endothelial cells |
| 6069 | GCUGCCCCC | | CUAGCCC | | |
| hsa-miR- | UUCUGCUGCCGGC | 29530 | GCCUUGGCCGGCAG | 31572 | Endothelial cells |
| 6071 | CAAGGC | | CAGAA | | |
| hsa-miR- | UCCUCAUCACACU | 29531 | CUAAGGUGCAGUGU | 31573 | Endothelial cells |
| 6072 | GCACCUUAG | | GAUGAGGA | | |
| hsa-miR- | GGUAGUGAGUUA | 29532 | GUAGCUGAUAACUC | 31574 | Endothelial cells |
| 6073 | UCAGCUAC | | ACUACC | | |
| hsa-miR- | GAUAUUCAGAGG | 29533 | CCACCUAGCCUCUG | 31575 | Endothelial cells |
| 6074 | CUAGGUGG | | AAUAUC | | |
| hsa-miR- | ACGGCCCAGGCGG | 29534 | CACCAAUGCCGCCU | 31576 | Endothelial cells |
| 6075 | CAUUGGUG | | GGGCCGU | | |
| hsa-miR- | AGCAUGACAGAG | 29535 | CCACCUCUCCUCUG | 31577 | Endothelial cells |
| 6076 | GAGAGGUGG | | UCAUGCU | | |
| hsa-miR- | GGGAAGAGCUGU | 29536 | GAAGGCCGUACAGC | 31578 | Endothelial cells |
| 6077 | ACGGCCUUC | | UCUUCCC | | |
| hsa-miR- | CCGCCUGAGCUAG | 29537 | CCACAGCUAGCUCA | 31579 | Endothelial cells |
| 6078 | CUGUGG | | GGCGG | | |
| hsa-miR- | UUGGAAGCUUGG | 29538 | CAGCUAGUUGGUCC | 31580 | Endothelial cells |
| 6079 | ACCAACUAGCUG | | AAGCUUCCAA | | |
| hsa-miR- | UCUAGUGCGGGC | 29539 | CGGGAACGCCCGCA | 31581 | Endothelial cells |
| 6080 | GUUCCCG | | CUAGA | | |
| hsa-miR- | AGGAGCAGUGCC | 29540 | GGCGCCUUGGCCGG | 31582 | Endothelial cells |
| 6081 | GGCCAAGGCGCC | | CACUGCUCCU | | |
| hsa-miR- | GAAUACGUCUGG | 29541 | GGAUCAACCAGACG | 31583 | Endothelial cells |
| 6082 | UUGAUCC | | UAUUC | | |
| hsa-miR- | CUUAUAUCAGAG | 29542 | CCCACAGCCUCUGA | 31584 | Endothelial cells |
| 6083 | GCUGUGGG | | UAUAAG | | |
| hsa-miR- | UUCCGCCAGUCGG | 29543 | CCGGCCACCGACUG | 31585 | Endothelial cells |
| 6084 | UGGCCGG | | GCGGAA | | |
| hsa-miR- | AAGGGGCUGGGG | 29544 | UGUGCUCCCCCAGC | 31586 | Endothelial cells |
| 6085 | GAGCACA | | CCCUU | | |
| hsa-miR- | AGGUUGGGAUCG | 29545 | AGCAUUGCAACCGA | 31587 | Endothelial cells |
| 92a-1-5p | GUUGCAAUGCU | | UCCCAACCU | | |
| hsa-miR- | GGGUGGGGAUUU | 29546 | GUAAUGCAACAAAU | 31588 | Endothelial cells |
| 92a-2-5p | GUUGCAUUAC | | CCCCACCC | | |
| hsa-miR- | UAUUGCACUUGU | 29547 | ACAGGCCGGGACAA | 31589 | Endothelial cells |
| 92a-3p | CCCGGCCUGU | | GUGCAAUA | | and CNS |
| hsa-miR- | UAUUGCACUCGUC | 29548 | GGAGGCCGGGACGA | 31590 | Endothelial cells |
| 92b-3p | CCGGCCUCC | | GUGCAAUA | | and heart |
| hsa-miR- | AGGGACGGGACG | 29549 | CACUGCACCGCGUC | 31591 | Endothelial cells |
| 92b-5p | CGGUGCAGUG | | CCGUCCCU | | and heart |
| hsa-miR- | AUCACAUUGCCAG | 29550 | GGAAAUCCCUGGCA | 31592 | Endothelial cells, |
| 23a-3p | GGAUUUCC | | AUGUGAU | | brain(astrocyte), |
| | | | | | blood(erythroid) |
| hsa-miR- | GGGGUUCCUGGG | 29551 | AAAUCCCAUCCCCA | 31593 | Endothelial cells, |
| 23a-5p | GAUGGGAUUU | | GGAACCCC | | brain(astrocyte), |
| | | | | | blood(erythroid) |
| hsa-miR- | ACUGCAGUGAAG | 29552 | CUACAAGUGCCUUC | 31594 | Endothelial cells, |
| 17-3p | GCACUUGUAG | | ACUGCAGU | | embryonic stem |
| | | | | | cells |
| hsa-miR- | AGCUACAUUGUC | 29553 | GAAACCCAGCAGAC | 31595 | Endothelial cells, |
| 221-3p | UGCUGGGUUUC | | AAUGUAGCU | | immune cells |
| hsa-miR- | ACCUGGCAUACAA | 29554 | AAAUCUACAUUGUA | 31596 | Endothelial cells, |
| 221-5p | UGUAGAUUU | | UGCCAGGU | | immune cells |
| hsa-miR- | UCGUACCGUGAG | 29555 | CGCAUUAUUACUCA | 31597 | Endothelial cells, |
| 126-3p | UAAUAAUGCG | | CGGUACGA | | lung |
| hsa-miR- | CAUUAUUACUUU | 29556 | CGCGUACCAAAAGU | 31598 | Endothelial cells, |
| 126-5p | UGGUACGCG | | AAUAAUG | | lung |
| hsa-miR- | UACUCCAGAGGGC | 29557 | CAUGAGUGACGCCC | 31599 | Endothelial |
| 508-5p | GUCACUCAUG | | UCUGGAGUA | | progenitor cells |
| | | | | | (pcs) |
| hsa-miR- | UCCCACGUUGUGG | 29558 | CUGCUGGGCCACAA | 31600 | Endothelial |
| 662 | CCCAGCAG | | CGUGGGA | | progenitor cells, |
| | | | | | oocytes |
| hsa-miR- | CCUCACCACCCCU | 29559 | UGCAGGCAGAAGGG | 31601 | Epididymis |
| 6511b-3p | UCUGCCUGCA | | GUGGUGAGG | | |
| hsa-miR- | CUGCAGGCAGAA | 29560 | UGUCAGCCCCACUU | 31602 | Epididymis |
| 6511b-5p | GUGGGGCUGACA | | CUGCCUGCAG | | |
| hsa-miR- | CCAAACCAGUCGU | 29561 | CCACAGGCACGACU | 31603 | Epididymis |
| 6715a-3p | GCCUGUGG | | GGUUUGG | | |
| hsa-miR- | CUCAAACCGGCUG | 29562 | CCACAGGCACAGCC | 31604 | Epididy mis |
| 6715b-3p | UGCCUGUGG | | GGUUUGAG | | |
| hsa-miR- | ACAGGCACGACUG | 29563 | UGCCAAACCAGUCG | 31605 | Epididymis |
| 6715b-5p | GUUUGGCA | | UGCCUGU | | |
| hsa-miR- | UCCGAACUCUCCA | 29564 | GCAGAGGAAUGGAG | 31606 | Epididymis |
| 6716-3p | UUCCUCUGC | | AGUUCGGA | | |
| hsa-miR- | UGGGAAUGGGGG | 29565 | GGCCCUUACCCCCA | 31607 | Epididymis |
| 6716-5p | UAAGGGCC | | UUCCCA | | |
| hsa-miR- | AGGCGAUGUGGG | 29566 | UCUCUACAUCCCCA | 31608 | Epididymis |
| 6717-5p | GAUGUAGAGA | | CAUCGCCU | | |
| hsa-miR- | UAGUGGUCAGAG | 29567 | UCAUAAGCCCUCUG | 31609 | Epididymis |
| 6718-5p | GGCUUAUGA | | ACCACUA | | |
| hsa-miR- | UCUGACAUCAGU | 29568 | CAGGAGAAUCACUG | 31610 | Epididymis |
| 6719-3p | GAUUCUCCUG | | AUGUCAGA | | |
| hsa-miR- | CGCGCCUGCAGGA | 29569 | UCUACCAGUUCCUG | 31611 | Epididymis |
| 6720-3p | ACUGGUAGA | | CAGGCGCG | | |
| hsa-miR- | UGGGCAGGGGCU | 29570 | CUCCUACAAUAAGC | 31612 | Epididymis |
| 6721-5p | UAUUGUAGGAG | | CCCUGCCCA | | |
| hsa-miR- | UGCAGGGGUCGG | 29571 | CCUGGCCCACCCGA | 31613 | Epididymis |
| 6722-3p | GUGGGCCAGG | | CCCCUGCA | | |
| hsa-miR- | AGGCGCACCCGAC | 29572 | GCAUGUGGUCGGGU | 31614 | Epididymis |
| 6722-5p | CACAUGC | | GCGCCU | | |
| hsa-miR- | AUAGUCCGAGUA | 29573 | GCCCCGACGUUACU | 31615 | Epididymis |
| 6723-5p | ACGUCGGGGC | | CGGACUAU | | |
| hsa-miR- | CUGGGCCCGCGGC | 29574 | CCCCACGCCCGCCG | 31616 | Epididy mis |
| 6724-5p | GGGCGUGGGG | | CGGGCCCAG | | |
| hsa-miR- | UACUUGGAAAGG | 29575 | CAACUGAUGCCUUU | 31617 | Epididy mis |
| 890 | CAUCAGUUG | | CCAAGUA | | |
| hsa-miR- | UGCAACGAACCUG | 29576 | UCAGUGGCUCAGGU | 31618 | Epididy mis |
| 891a | AGCCACUGA | | UCGUUGCA | | |
| hsa-miR- | UGCAACUUACCUG | 29577 | UCAAUGACUCAGGU | 31619 | Epididymis |
| 891b | AGUCAUUGA | | AAGUUGCA | | |
| hsa-miR- | CACUGUGUCCUUU | 29578 | CUACGCAGAAAGGA | 31620 | Epididymis |
| 892a | CUGCGUAG | | CACAGUG | | |
| hsa-miR- | CACUGGCUCCUUU | 29579 | UCUACCCAGAAAGG | 31621 | Epididymis |
| 892b | CUGGGUAGA | | AGCCAGUG | | |
| hsa-miR- | CACUGUUUCCUUU | 29580 | UCCACUCAGAAAGG | 31622 | Epididy mis |
| 892c-3p | CUGAGUGGA | | AAACAGUG | | |
| hsa-miR- | UAUUCAGAAAGG | 29581 | UGACUGGCACCUUU | 31623 | Epididymis |
| 892c-5p | UGCCAGUCA | | CUGAAUA | | |
| hsa-miR- | AUUCCUAGAAAU | 29582 | UAUGAACAAUUUCU | 31624 | Epithelial cells |
| 384 | UGUUCAUA | | AGGAAU | | |
| hsa-miR- | UAAUACUGUCUG | 29583 | ACGGUUUUACCAGA | 31625 | Epithelial cells |
| 429 | GUAAAACCGU | | CAGUAUUA | | |
| hsa-miR- | UGAAACAUACAC | 29584 | GAGGUUUCCCGUGU | 31626 | Epithelial cells |
| 494 | GGGAAACCUC | | AUGUUUCA | | |
| hsa-let- | CUAUACAACCUAC | 29585 | GGGAAGGCAGUAGG | 31627 | Epithelial cells, |
| 7b-3p | UGCCUUCCC | | UUGUAUAG | | endothelial cells |
| | | | | | (vascular) |
| hsa-let- | UGAGGUAGUAGG | 29586 | AACCACACAACCUA | 31628 | Epithelial cells, |
| 7b-5p | UUGUGUGGUU | | CUACCUCA | | endothelial cells |
| | | | | | (vascular) |
| hsa-miR- | UAACACUGUCUG | 29587 | ACAUCGUUACCAGA | 31629 | Epithelial cells, |
| 200a-3p | GUAACGAUGU | | CAGUGUUA | | many other tissues |
| hsa-miR- | CAUCUUACCGGAC | 29588 | UCCAGCACUGUCCG | 31630 | Epithelial cells, |
| 200a-5p | AGUGCUGGA | | GUAAGAUG | | many other tissues |
| hsa-miR- | UAAUACUGCCUG | 29589 | UCAUCAUUACCAGG | 31631 | Epithelial cells, |
| 200b-3p | GUAAUGAUGA | | CAGUAUUA | | many other tissues |
| hsa-miR- | CAUCUUACUGGGC | 29590 | UCCAAUGCUGCCCA | 31632 | Epithelial cells, |
| 200b-5p | AGCAUUGGA | | GUAAGAUG | | many other tissues |
| hsa-miR- | UAAUACUGCCGG | 29591 | UCCAUCAUUACCCG | 31633 | Epithelial cells, |
| 200c-3p | GUAAUGAUGGA | | GCAGUAUUA | | many other tissues, |
| | | | | | embryonic stem |
| | | | | | cells |
| hsa-miR- | CGUCUUACCCAGC | 29592 | CCAAACACUGCUGG | 31634 | Epithelial cells, |
| 200c-5p | AGUGUUUGG | | GUAAGACG | | many other tissues, |
| | | | | | embryonic stem |
| | | | | | cells |
| hsa-miR- | UCCAGCAUCAGUG | 29593 | CAACAAAAUCACUG | 31635 | Epithelial cells, |
| 338-3p | AUUUUGUUG | | AUGCUGGA | | oligodendrocytes |
| hsa-miR- | UACAGUAUAGAU | 29594 | AGUACAUCAUCUAU | 31636 | Erythroid |
| 144-3p | GAUGUACU | | ACUGUA | | |
| hsa-miR- | GGAUAUCAUCAU | 29595 | CUUACAGUAUAUGA | 31637 | Erythroid |
| 144-5p | AUACUGUAAG | | UGAUAUCC | | |
| hsa-miR- | CGGGGCAGCUCAG | 29596 | AUCCUGUACUGAGC | 31638 | Erythroid cells |
| 486-3p | UACAGGAU | | UGCCCCG | | |
| hsa-miR- | UCCUUCUGCUCCG | 29597 | CUGGGGGACGGAGC | 31639 | Esophageal cell line |
| 1237-3p | UCCCCCAG | | AGAAGGA | | KYSE-150R |
| hsa-miR- | CGGGGGCGGGGCC | 29598 | CGCGCUUCGGCCCC | 31640 | Esophageal cell line |
| 1237-5p | GAAGCGCG | | GCCCCCG | | KYSE-150R |
| hsa-miR- | UCCUGCGCGUCCC | 29599 | GGGCAUCUGGGACG | 31641 | Esophageal cell line |
| 1539 | AGAUGCCC | | CGCAGGA | | KYSE-150R |
| hsa-miR- | CAUCAGAAUUCA | 29600 | CUAGCCUCCAUGAA | 31642 | Female |
| 2115-3p | UGGAGGCUAG | | UUCUGAUG | | reproductive tract |
| hsa-miR- | AGCUUCCAUGACU | 29601 | UCCAUCAGGAGUCA | 31643 | Female |
| 2115-5p | CCUGAUGGA | | UGGAAGCU | | reproductive tract |
| hsa-miR- | UGACAGCGCCCUG | 29602 | GAGCCAGGCAGGGC | 31644 | Female |
| 2277-3p | CCUGGCUC | | GCUGUCA | | reproductive tract |
| hsa-miR- | AGCGCGGGCUGA | 29603 | GACUGGCAGCGCUC | 31645 | Female |
| 2277-5p | GCGCUGCCAGUC | | AGCCCGCGCU | | reproductive tract |
| hsa-miR- | CUGGGAGGUGUG | 29604 | ACCACGAUAUCACA | 31646 | Female |
| 3689a-3p | AUAUCGUGGU | | CCUCCCAG | | reproductive tract |
| hsa-miR- | UGUGAUAUCAUG | 29605 | UCCCAGGAACCAUG | 31647 | Female |
| 3689b-5p | GUUCCUGGGA | | AUAUCACA | | reproductive tract |
| hsa-miR- | AGGUGCUCCAGGC | 29606 | UGUGAGCCAGCCUG | 31648 | Female |
| 3907 | UGGCUCACA | | GAGCACCU | | reproductive tract |
| hsa-miR- | GAGCAAUGUAGG | 29607 | AAACAGUCUACCUA | 31649 | Female |
| 3908 | UAGACUGUUU | | CAUUGCUC | | reproductive tract |
| hsa-miR- | UGUCCUCUAGGGC | 29608 | AGACUGCAGGCCCU | 31650 | Female |
| 3909 | CUGCAGUCU | | AGAGGACA | | reproductive tract |
| hsa-miR- | AAAGGCAUAAAA | 29609 | UGUCUUGGUUUUAU | 31651 | Female |
| 3910 | CCAAGACA | | GCCUUU | | reproductive tract |
| hsa-miR- | UAACGCAUAAUA | 29610 | ACAUGUCCAUAUUA | 31652 | Female |
| 3912 | UGGACAUGU | | UGCGUUA | | reproductive tract |
| hsa-miR- | UUGAGGAAAAGA | 29611 | AAUAAGACCAUCUU | 31653 | Female |
| 3915 | UGGUCUUAUU | | UUCCUCAA | | reproductive tract |
| hsa-miR- | AAGAGGAAGAAA | 29612 | CUGAGAACCAGCCA | 31654 | Female |
| 3916 | UGGCUGGUUCUC | | UUUCUUCCUCUU | | reproductive tract |
| | AG | | | | |
| hsa-miR- | GCUCGGACUGAGC | 29613 | CCCACCUGCUCAGU | 31655 | Female |
| 3917 | AGGUGGG | | CCGAGC | | reproductive tract |
| hsa-miR- | ACAGGGCCGCAGA | 29614 | AGUCUCCAUCUGOG | 31656 | Female |
| 3918 | UGGAGACU | | GCCCUGU | | reproductive tract |
| hsa-miR- | GCAGAGAACAAA | 29615 | ACUGAGUCCUUUGU | 31657 | Female |
| 3919 | GGACUCAGU | | UCUCUGC | | reproductive tract |
| hsa-miR- | ACUGAUUAUCUU | 29616 | UCAGAGAGUUAAGA | 31658 | Female |
| 3920 | AACUCUCUGA | | UAAUCAGU | | reproductive tract |
| hsa-miR- | UCUCUGAGUACCA | 29617 | ACAAGGCAUAUGGU | 31659 | Female |
| 3921 | UAUGCCUUGU | | ACUCAGAGA | | reproductive tract |
| hsa-miR- | AACUAGUAAUGU | 29618 | CCCUAAUCCAACAU | 31660 | Female |
| 3923 | UGGAUUAGGG | | UACUAGUU | | reproductive tract |
| hsa-miR- | AUAUGUAUAUGU | 29619 | AGUAGCAGUCACAU | 31661 | Female |
| 3924 | GACUGCUACU | | AUACAUAU | | reproductive tract |
| hsa-miR- | UGGCCAAAAAGC | 29620 | UCUCUGCCUGCUUU | 31662 | Female |
| 3926 | AGGCAGAGA | | UUGGCCA | | reproductive tract |
| hsa-miR- | GGAGGAACCUUG | 29621 | GCCGAAGCUCCAAG | 31663 | Female |
| 3928 | GAGCUUCGGC | | GUUCCUCC | | reproductive tract |
| hsa-miR- | GAGGCUGAUGUG | 29622 | AGUGGUCUACUCAC | 31664 | Female |
| 3929 | AGUAGACCACU | | AUCAGCCUC | | reproductive tract |
| hsa-miR- | CUGUCCUAAGGU | 29623 | AACUCAACAACCUU | 31665 | Female |
| 676-3p | UGUUGAGUU | | AGGACAG | | reproductive tract |
| hsa-miR- | UCUUCAACCUCAG | 29624 | UGCAAGUCCUGAGG | 31666 | Female |
| 676-5p | GACUUGCA | | UUGAAGA | | reproductive tract |
| hsa-miR- | UGUGAUAUCAUG | 29625 | UCCCAGGAACCAUG | 31667 | Female |
| 3689a-5p | GUUCCUGGGA | | AUAUCACA | | reproductive tract |
| | | | | | and peripheral |
| | | | | | blood |
| hsa-miR- | CUGGGAGGUGUG | 29626 | ACCACAAUAUCACA | 31668 | Female |
| 3689b-3p | AUAUUGUGGU | | CCUCCCAG | | reproductive tract |
| | | | | | and peripheral |
| | | | | | blood |
| hsa-miR- | CAGGUAGAUAUU | 29627 | AUGCCUAUCAAAUA | 31669 | Female |
| 3927-3p | UGAUAGGCAU | | UCUACCUG | | reproductive tract |
| | | | | | and psoriasis |
| hsa-miR- | GCCUAUCACAUAU | 29628 | ACAGGCAGAUAUGU | 31670 | Female |
| 3927-5p | CUGCCUGU | | GAUAGGC | | reproductive tract |
| | | | | | and psoriasis |
| hsa-miR- | GGGCUGGGGCGC | 29629 | ACCUCCCCGCGCCC | 31671 | Fibroblast |
| 5787 | GGGGAGGU | | CAGCCC | | |
| hsa-miR- | UGCUUCCUUUCAG | 29630 | ACCCUCUGAAAGGA | 31672 | Frontal cortex |
| 516a-3p | AGGGU | | AGCA | | |
| hsa-miR- | UGAGUUGGCCAU | 29631 | CUCACUCAGAUGGC | 31673 | Frontal cortex |
| 571 | CUGAGUGAG | | CAACUCA | | |
| hsa-miR- | AACAUUCAUUGU | 29632 | ACCCACCGACAACA | 31674 | Glia cells |
| 181d | UGUCGGUGGGU | | AUGAAUGUU | | |
| hsa-miR- | UCACAGUGAACCG | 29633 | AAAGAGACCGGUUC | 31675 | Glioblast, brain |
| 128 | GUCUCUUU | | ACUGUGA | | |
| hsa-miR- | CAACAAAUCACAG | 29634 | UAUGGCAGACUGUG | 31676 | Glioblast, brain, |
| 7-1-3p | UCUGCCAUA | | AUUUGUUG | | pancreas |
| hsa-miR- | CUCACUGAACAAU | 29635 | UUGCAUUCAUUGUU | 31677 | Glioblast, |
| 181b-3p | GAAUGCAA | | CAGUGAG | | Embryonic stem |
| | | | | | cells, epidermal |
| | | | | | (keratinocytes) |
| hsa-miR- | AACAUUCAUUGC | 29636 | ACCCACCGACAGCA | 31678 | Glioblast, |
| 181b-5p | UGUCGGUGGGU | | AUGAAUGUU | | Embryonic stem |
| | | | | | cells, epidermal |
| | | | | | (keratinocytes) |
| hsa-miR- | ACCAUCGACCGUU | 29637 | GGUACAAUCAACGG | 31679 | Glioblast, myeloid |
| 181a-3p | GAUUGUACC | | UCGAUGGU | | cells, embryonic |
| | | | | | stem cells |
| hsa-miR- | AACAUUCAACGCU | 29638 | ACUCACCGACAGCG | 31680 | Glioblast, myeloid |
| 181a-5p | GUCGGUGAGU | | UUGAAUGUU | | cells, embryonic |
| | | | | | stem cells |
| hsa-miR- | ACCACUGACCGUU | 29639 | GGUACAGUCAACGG | 31681 | Glioblast, stem cells |
| 181a-2-3p | GACUGUACC | | UCAGUGGU | | |
| hsa-miR- | CUGUUGCCACUAA | 29640 | AGGUUGAGGUUAGU | 31682 | Heart |
| 744-3p | CCUCAACCU | | GGCAACAG | | |
| hsa-miR- | AUAAGACGAGCA | 29641 | ACAAGCUUUUUGCU | 31683 | Heart |
| 208a | AAAAGCUUGU | | CGUCUUAU | | (cardiomyocyte), |
| | | | | | muscle |
| hsa-miR- | AUAAGACGAACA | 29642 | ACAAACCUUUUGUU | 31684 | Heart |
| 208b | AAAGGUUUGU | | CGUCUUAU | | (cardiomyocyte), |
| | | | | | muscle |
| hsa-miR- | AGGGAGGGACGG | 29643 | GCACAGCCCCCGUC | 31685 | Heart and brain |
| 149-3p | GGGCUGUGC | | CCUCCCU | | |
| hsa-miR- | UCUGGCUCCGUGU | 29644 | GGGAGUGAAGACAC | 31686 | Heart and brain |
| 149-5p | CUUCACUCCC | | GGAGCCAGA | | |
| hsa-miR-1 | UGGAAUGUAAAG | 29645 | AUACAUACUUCUUU | 31687 | Heart and muscle |
| | AAGUAUGUAU | | ACAUUCCA | | |
| hsa-miR- | AACAUCACAGCAA | 29646 | AGCACAGACUUGCU | 31688 | Heart, cardiac stem |
| 499a-3p | GUCUGUGCU | | GUGAUGUU | | cells |
| hsa-miR- | UUAAGACUUGCA | 29647 | AAACAUCACUGCAA | 31689 | Heart, cardiac stem |
| 499a-5p | GUGAUGUUU | | GUCUUAA | | cells |
| hsa-miR- | AACAUCACUGCAA | 29648 | UGUUAAGACUUGCA | 31690 | Heart, cardiac stem |
| 499b-3p | GUCUUAACA | | GUGAUGUU | | cells |
| hsa-miR- | ACAGACUUGCUG | 29649 | UGAACAUCACAGCA | 31691 | Heart, cardiac stem |
| 499b-5p | UGAUGUUCA | | AGUCUGU | | cells |
| hsa-miR- | AAACCGUUACCAU | 29650 | AACUCAGUAAUGGU | 31692 | Heart, central |
| 45la | UACUGAGUU | | AACGGUUU | | nervous system, |
| | | | | | epithelial cells |
| hsa-miR- | UAGCAAGAGAAC | 29651 | AAUGGUAAUGGUUC | 31693 | Heart, central |
| 451b | CAUUACCAUU | | UCUUGCUA | | nervous system, |
| | | | | | epithelial cells |
| hsa-miR- | UGAGGGGCAGAG | 29652 | AAAGUCUCGCUCUC | 31694 | Heart, embryonic |
| 423-5p | AGCGAGACUUU | | UGCCCCUCA | | stem cells |
| hsa-miR- | CAAGCUCGCUUCU | 29653 | CAGACCCAUAGAAG | 31695 | Hematopoietic cells |
| 99a-3p | AUGGGUCUG | | CGAGCUUG | | |
| hsa-miR- | AACCCGUAGAUCC | 29654 | CACAAGAUCGGAUC | 31696 | Hematopoietic cells |
| 99a-5p | GAUCUUGUG | | UACGGGUU | | |
| hsa-miR- | CAAGCUCGUGUCU | 29655 | CGGACCCACAGACA | 31697 | Hematopoietic cells |
| 99b-3p | GUGGGUCCG | | CGAGCUUG | | and embryonic stem |
| | | | | | cells |
| hsa-miR- | CACCCGUAGAACC | 29656 | CGCAAGGUCGGUUC | 31698 | Hematopoietic cells |
| 99b-5p | GACCUUGCG | | UACGGGUG | | and embryonic stem |
| | | | | | cells |
| hsa-miR- | UGGAGACGCGGCC | 29657 | ACUCCAACAGGGCC | 31699 | Hematocytes, brain |
| 139-3p | CUGUUGGAGU | | GCGUCUCCA | | |
| hsa-miR- | UCUACAGUGCACG | 29658 | ACUGGAGACACGUG | 31700 | Hematocytes, brain |
| 139-5p | UGUCUCCAGU | | CACUGUAGA | | |
| hsa-miR- | CAAAUUCGUAUC | 29659 | UAUUCCCCUAGAUA | 31701 | Hematopoietic cells |
| 10a-3p | UAGGGGAAUA | | CGAAUUUG | | |
| hsa-miR- | UACCCUGUAGAUC | 29660 | CACAAAUUCGGAUC | 31702 | Hematopoietic cells |
| 10a-5p | CGAAUUUGUG | | UACAGGGUA | | |
| hsa-miR- | CCUGCAGCGACUU | 29661 | GGAAGCCAUCAAGU | 31703 | Hematopoietic cells |
| 1184 | GAUGGCUUCC | | CGCUGCAGG | | |
| hsa-miR- | UCAGUGCACUACA | 29662 | ACAAAGUUCUGUAG | 31704 | Hematopoietic cells |
| 148a-3p | GAACUUUGU | | UGCACUGA | | |
| hsa-miR- | AAAGUUCUGAGA | 29663 | AGUCGGAGUGUCUC | 31705 | Hematopoietic cells |
| 148a-5p | CACUCCGACU | | AGAACUUU | | |
| hsa-miR- | CCUGUUCUCCAUU | 29664 | GAGCCAAGUAAUGG | 31706 | Hematopoietic cells |
| 26b-3p | ACUUGGCUC | | AGAACAGG | | |
| hsa-miR- | UUCAAGUAAUUC | 29665 | ACCUAUCCUGAAUU | 31707 | Hematopoietic cells |
| 26b-5p | AGGAUAGGU | | ACUUGAA | | |
| hsa-miR- | GCCCUGAACGAGG | 29666 | CUCCAGACCCCUCG | 31708 | Hematopoietic cells |
| 345-3p | GGUCUGGAG | | UUCAGGGC | | |
| hsa-miR- | GCUGACUCCUAGU | 29667 | GAGCCCUGGACUAG | 31709 | Hematopoietic cells |
| 345-5p | CCAGGGCUC | | GAGUCAGC | | |
| hsa-miR- | AUCACACAAAGGC | 29668 | ACAAAAGUUGCCUU | 31710 | Hematopoietic cells |
| 377-3p | AACUUUUGU | | UGUGUGAU | | |
| hsa-miR- | AGAGGUUGCCCU | 29669 | GAAUUCACCAAGGG | 31711 | Hematopoietic cells |
| 377-5p | UGGUGAAUUC | | CAACCUCU | | |
| hsa-miR- | GGUAGAUUUUCC | 29670 | ACCAUAGAAGGAAA | 31712 | Hematopoietic cells |
| 376a-2-5p | UUCUAUGGU | | AUCUACC | | (erythroid, platelet, |
| | | | | | and lymphoma) |
| hsa-miR- | AUCAUAGAGGAA | 29671 | ACGUGGAUUUUCCU | 31713 | Hematopoietic cells |
| 376a-3p | AAUCCACGU | | CUAUGAU | | (erythroid, platelet, |
| | | | | | and lymphoma) |
| hsa-miR- | GUAGAUUCUCCU | 29672 | UACUCAUAGAAGGA | 31714 | Hematopoietic cells |
| 376a-5p | UCUAUGAGUA | | GAAUCUAC | | (erythroid, platelet, |
| | | | | | and lymphoma) |
| hsa-miR- | CUGGUACAGGCCU | 29673 | CUGUCCCCCAGGCC | 31715 | Hematopoietic cells |
| 150-3p | GGGGGACAG | | UGUACCAG | | (lymphoid) |
| hsa-miR- | UCUCCCAACCCUU | 29674 | CACUGGUACAAGGG | 31716 | Hematopoietic cells |
| 150-5p | GUACCAGUG | | UUGGGAGA | | (lymphoid) |
| hsa-miR- | ACGGGUUAGGCU | 29675 | AGCUCCCAAGAGCC | 31717 | Hematopoietic cells |
| 125b-1-3p | CUUGGGAGCU | | UAACCCGU | | (monocytes), |
| | | | | | brain(neuron) |
| hsa-miR- | UCACAAGUCAGGC | 29676 | GUCCCAAGAGCCUG | 31718 | Hematopoietic cells |
| 125b-2-3p | UCUUGGGAC | | ACUUGUGA | | (monocytes), |
| | | | | | brain(neuron) |
| hsa-miR- | AACCCGUAGAUCC | 29677 | CACAAGUUCGGAUC | 31719 | Hematopoietic cells |
| 100-5p | GAACUUGUG | | UACGGGUU | | and endothelial |
| | | | | | cells |
| hsa-let- | CUGUACAGGCCAC | 29678 | GCAAGGCAGUGGCC | 31720 | Hematopoietic |
| 7g-3p | UGCCUUGC | | UGUACAG | | cells, adipose, |
| | | | | | smooth muscle cells |
| hsa-let- | UGAGGUAGUAGU | 29679 | AACUGUACAAACUA | 31721 | Hematopoietic |
| 7g-5p | UUGUACAGUU | | CUACCUCA | | cells, adipose, |
| | | | | | smooth muscle cells |
| hsa-miR- | UCCCUGAGACCCU | 29680 | UCACAAGUUAGGGU | 31722 | Hematopoietic |
| 125b-5p | AACUUGUGA | | CUCAGGGA | | cells, brain (neuron) |
| hsa-miR- | CAAGCUUGUAUC | 29681 | CAUACCUAUAGAUA | 31723 | Hematopoietic |
| 100-3p | UAUAGGUAUG | | CAAGCUUG | | cells, endothelial |
| | | | | | cells |
| hsa-miR- | AAAGUGCUGCGA | 29682 | ACGCUCAAAUGUCG | 31724 | Hematopoietic |
| 372 | CAUUUGAGCGU | | CAGCACUUU | | cells, lung, |
| | | | | | placental (blood) |
| hsa-miR- | UUUAGAGACGGG | 29683 | AGAGCAAGACCCCG | 31725 | Hepatocyte |
| 1303 | GUCUUGCUCU | | UCUCUAAA | | |
| hsa-miR- | UGGCCCUGACUGA | 29684 | ACUGCUGGUCUUCA | 31726 | Hepatocytes |
| 1291 | AGACCAGCAGU | | GUCAGGGCCA | | |
| hsa-miR- | GCGACCCAUACUU | 29685 | CUGAAACCAAGUAU | 31727 | Hepatocytes |
| 551b-3p | GGUUUCAG | | GGGUCGC | | |
| hsa-miR- | GAAAUCAAGCGU | 29686 | GGUCUCACCCACGC | 31728 | Hepatocytes |
| 551b-5p | GGGUGAGACC | | UUGAUUUC | | |
| hsa-miR- | CCCUGGGCCUCUG | 29687 | CUGGGGAGCAGAGG | 31729 | Hepatocytes |
| 939-3p | CUCCCCAG | | CCCAGGG | | |
| hsa-miR- | UGGGGAGCUGAG | 29688 | CACCCCCAGAGCCU | 31730 | Hepatocytes |
| 939-5p | GCUCUGGGGGUG | | CAGCUCCCCA | | |
| hsa-miR- | GGGGAGCUGUGG | 29689 | UACUGCUUCCACAG | 31731 | Human testis |
| 920 | AAGCAGUA | | CUCCCC | | |
| hsa-miR- | CUAGUGAGGGAC | 29690 | GAAUCCUGGUUCUG | 31732 | Human testis |
| 921 | AGAACCAGGAUU | | UCCCUCACUAG | | |
| | C | | | | |
| hsa-miR- | AGAGUCUUGUGA | 29691 | GCAAGACAUCACAA | 31733 | Human testis |
| 924 | UGUCUUGC | | GACUCU | | |
| hsa-miR- | GCAGCAGAGAAU | 29692 | GACGUAGUCCUAUU | 31734 | Human testis, |
| 922 | AGGACUACGUC | | CUCUGCUGC | | neuronal tissues |
| hsa-miR- | UGAGCGCCUCGAC | 29693 | CGGCUCUGUCGUCG | 31735 | Immune cell |
| 339-3p | GACAGAGCCG | | AGGCGCUCA | | |
| hsa-miR- | UCCCUGUCCUCCA | 29694 | CGUGAGCUCCUGGA | 31736 | Immune cell |
| 339-5p | GGAGCUCACG | | GGACAGGGA | | |
| hsa-let- | CUAUACGGCCUCC | 29695 | GGAAAGCUAGGAGG | 31737 | Immune cells |
| 7e-3p | UAGCUUUCC | | CCGUAUAG | | |
| hsa-let- | UGAGGUAGGAGG | 29696 | AACUAUACAACCUC | 31738 | Immune cells |
| 7e-5p | UUGUAUAGUU | | CUACCUCA | | |
| hsa-let- | CUGCGCAAGCUAC | 29697 | AGCAAGGCAGUAGC | 31739 | Immune cells |
| 7i-3p | UGCCUUGCU | | UUGCGCAG | | |
| hsa-let- | UGAGGUAGUAGU | 29698 | AACAGCACAAACUA | 31740 | Immune cells |
| 7i-5p | UUGUGCUGUU | | CUACCUCA | | |
| hsa-miR- | UGCCCUGUGGACU | 29699 | CCAGAACUGAGUCC | 31741 | Immune cells |
| 146b-3p | CAGUUCUGG | | ACAGGGCA | | |
| hsa-miR- | UGGUUCUAGACU | 29700 | UAGUUGGCAAGUCU | 31742 | Immune cells |
| 182-3p | UGCCAACUA | | AGAACCA | | |
| hsa-miR- | UGUCUGCCCGCAU | 29701 | AGAGGCAGGCAUGC | 31743 | Immune cells |
| 346 | GCCUGCCUCU | | GGGCAGACA | | |
| hsa-miR- | AAAAGUAAUUGC | 29702 | ACCAAAGACCGCAA | 31744 | Immune cells |
| 548j | GGUCUUUGGU | | UUACUUUU | | |
| hsa-miR- | UCGAGGAGCUCAC | 29703 | AGACUGUGAGCUCC | 31745 | Immune cells (B- |
| 151b | AGUCU | | UCGA | | cells) |
| hsa-let- | CUAUACAAUCUA | 29704 | GGGAAGGCAAUAGA | 31746 | Immune cells (T |
| 7f-1-3p | UUGCCUUCCC | | UUGUAUAG | | cells) |
| hsa-let- | CUAUACAGUCUAC | 29705 | GGAAAGACAGUAGA | 31747 | Immune cells (T |
| 7f-2-3p | UGUCUUUCC | | CUGUAUAG | | cells) |
| hsa-let- | UGAGGUAGUAGA | 29706 | AACUAUACAAUCUA | 31748 | Immune cells (T |
| 7f-5p | UUGUAUAGUU | | CUACCUCA | | cells) |
| hsa-miR- | AAAAGUAAUUGU | 29707 | GGCCAAAACCACAA | 31749 | Immune cells, |
| 548b-5p | GGUUUUGGCC | | UUACUUUU | | frontal cortex |
| hsa-miR- | AAAAGUAAUUGC | 29708 | GGCAAAAACCGCAA | 31750 | Immune cells, |
| 548c-5p | GGUUUUUGCC | | UUACUUUU | | frontal cortex |
| hsa-miR- | CCUCUGAAAUUCA | 29709 | CUGAAGAACUGAAU | 31751 | Immune cells, |
| 146a-3p | GUUCUUCAG | | UUCAGAGG | | hematopoiesis |
| hsa-miR- | UGAGAACUGAAU | 29710 | AACCCAUGGAAUUC | 31752 | Immune cells, |
| 146a-5p | UCCAUGGGUU | | AGUUCUCA | | hematopoiesis |
| hsa-miR- | ACAGCAGGCACAG | 29711 | ACUGCCUGUCUGUG | 31753 | Immune cells, |
| 214-3p | ACAGGCAGU | | CCUGCUGU | | pancreas |
| hsa-miR- | UGCCUGUCUACAC | 29712 | GCACAGCAAGUGUA | 31754 | Immune cells, |
| 214-5p | UUGCUGUGC | | GACAGGCA | | pancreas |
| hsa-miR- | UAGCACCAUCUGA | 29713 | UAACCGAUUUCAGA | 31755 | Immune system |
| 29a-3p | AAUCGGUUA | | UGGUGCUA | | |
| hsa-miR- | ACUGAUUUCUUU | 29714 | CUGAACACCAAAAG | 31756 | Immune system |
| 29a-5p | UGGUGUUCAG | | AAAUCAGU | | |
| hsa-miR- | GCUGGUUUCAUA | 29715 | UCUAAACCACCAUA | 31757 | Immune system |
| 29b-1-5p | UGGUGGUUUAGA | | UGAAACCAGC | | |
| hsa-miR- | CUGGUUUCACAU | 29716 | CUAAGCCACCAUGU | 31758 | Immune system |
| 29b-2-5p | GGUGGCUUAG | | GAAACCAG | | |
| hsa-miR- | UAGCACCAUUUG | 29717 | AACACUGAUUUCAA | 31759 | Immune system |
| 29b-3p | AAAUCAGUGUU | | AUGGUGCUA | | |
| hsa-miR- | UAGCACCAUUUG | 29718 | UAACCGAUUUCAAA | 31760 | Immune system |
| 29c-3p | AAAUCGGUUA | | UGGUGCUA | | |
| hsa-miR- | UGACCGAUUUCUC | 29719 | GAACACCAGGAGAA | 31761 | Immune system |
| 29c-5p | CUGGUGUUC | | AUCGGUCA | | |
| hsa-miR- | UGUCACUCGGCUC | 29720 | GUAGUGGGCCGAGC | 31762 | Keratinocytes |
| 668 | GGCCCACUAC | | CGAGUGACA | | |
| hsa-miR- | CUGCCAAUUCCAU | 29721 | CUGUGACCUAUGGA | 31763 | Kidney |
| 192-3p | AGGUCACAG | | AUUGGCAG | | |
| hsa-miR- | CUGACCUAUGAA | 29722 | GGCUGUCAAUUCAU | 31764 | Kidney |
| 192-5p | UUGACAGCC | | AGGUCAG | | |
| hsa-miR- | ACUGCCCCAGGUG | 29723 | CCAGCAGCACCUGG | 31765 | Kidney |
| 324-3p | CUGCUGG | | GGCAGU | | |
| hsa-miR- | AACGCCAUUAUCA | 29724 | UAUUUAGUGUGAUA | 31766 | Kidney and |
| 122-3p | CACUAAAUA | | AUGGCGUU | | liver/hepatocytes |
| hsa-miR- | CUUUCAGUCGGA | 29725 | GCUGCAAACAUCCG | 31767 | Kidney and |
| 30a-3p | UGUUUGCAGC | | ACUGAAAG | | pancreatic cells |
| hsa-miR- | AAUUGCACGGUA | 29726 | UACAGAUGGAUACC | 31768 | Kidney stem cell, |
| 363-3p | UCCAUCUGUA | | GUGCAAUU | | blood cells |
| hsa-miR- | CGGGUGGAUCAC | 29727 | AAAUUGCAUCGUGA | 31769 | Kidney stem cell, |
| 363-5p | GAUGCAAUUU | | UCCACCCG | | blood cells |
| hsa-miR- | CUGGGAGGUGGA | 29728 | GAAGUAAACAUCCA | 31770 | Kidney, adipose, |
| 30b-3p | UGUUUACUUC | | CCUCCCAG | | CNS(prefrontal |
| | | | | | cortex) |
| hsa-miR- | UGUAAACAUCCU | 29729 | AGCUGAGUGUAGGA | 31771 | Kidney, adipose, |
| 30b-5p | ACACUCAGCU | | UGUUUACA | | CNS(prefrontal |
| | | | | | cortex) |
| hsa-miR- | CUGGGAGAGGGU | 29730 | GGAGUAAACAACCC | 31772 | Kidney, adipose, |
| 30c-1-3p | UGUUUACUCC | | UCUCCCAG | | CNS(prefrontal |
| | | | | | cortex) |
| hsa-miR- | CUGGGAGAAGGC | 29731 | AGAGUAAACAGCCU | 31773 | Kidney, adipose, |
| 30c-2-3p | UGUUUACUCU | | UCUCCCAG | | CNS(prefrontal |
| | | | | | cortex) |
| hsa-miR- | UGUAAACAUCCU | 29732 | GCUGAGAGUGUAGG | 31774 | Kidney, adipose, |
| 30c-5p | ACACUCUCAGC | | AUGUUUACA | | CNS(prefrontal |
| | | | | | cortex) |
| hsa-miR- | UUUUUCAUUAUU | 29733 | GGUCAGGAGCAAUA | 31775 | Kidney, breast |
| 335-3p | GCUCCUGACC | | AUGAAAAA | | |
| hsa-miR- | UCAAGAGCAAUA | 29734 | ACAUUUUUCGUUAU | 31776 | Kidney, breast |
| 335-5p | ACGAAAAAUGU | | UGCUCUUGA | | |
| hsa-miR- | CAAAGUGCUUAC | 29735 | CUACCUGCACUGUA | 31777 | Kidney, breast and |
| 17-5p | AGUGCAGGUAG | | AGCACUUUG | | endothelial cells |
| hsa-miR- | GGAUUCCUGGAA | 29736 | AGAACAGUAUUUCC | 31778 | Kidney, cartilage, |
| 145-3p | AUACUGUUCU | | AGGAAUCC | | vascular smooth |
| | | | | | muscle |
| hsa-miR- | GUCCAGUUUUCCC | 29737 | AGGGAUUCCUGGGA | 31779 | Kidney, cartilage, |
| 145-5p | AGGAAUCCCU | | AAACUGGAC | | vascular smooth |
| | | | | | muscle |
| hsa-miR- | ACUGCAUUAUGA | 29738 | CUUUAAGUGCUCAU | 31780 | Kidney, endothelial |
| 20a-3p | GCACUUAAAG | | AAUGCAGU | | cells, osteogenic |
| | | | | | cells |
| hsa-miR- | UAAAGUGCUUAU | 29739 | CUACCUGCACUAUA | 31781 | Kidney, endothelial |
| 20a-5p | AGUGCAGGUAG | | AGCACUUUA | | cells, osteogenic |
| | | | | | cells |
| hsa-miR- | GAGGGUUGGGUG | 29740 | GGAGAGCCUCCACC | 31782 | Kidney, heart, lung, |
| 296-3p | GAGGCUCUCC | | CAACCCUC | | endothelial cells |
| hsa-miR- | CUGUGCGUGUGA | 29741 | UCAGCCGCUGUCAC | 31783 | Kidney, heart, |
| 210 | CAGCGGCUGA | | ACGCACAG | | vascular endothelial |
| | | | | | cells |
| hsa-miR- | CCAGUGGGGCUGC | 29742 | CAGAUAACAGCAGC | 31784 | Kidney, liver |
| 194-3p | UGUUAUCUG | | CCCACUGG | | |
| hsa-miR- | UGUAACAGCAAC | 29743 | UCCACAUGGAGUUG | 31785 | Kidney, liver |
| 194-5p | UCCAUGUGGA | | CUGUUACA | | |
| hsa-miR- | UCACAGUGGUCUC | 29744 | AUAAUCCCAGAGAC | 31786 | Kidney, pancreas |
| 216a-3p | UGGGAUUAU | | CACUGUGA | | |
| hsa-miR- | UAAUCUCAGCUG | 29745 | UCACAGUUGCCAGC | 31787 | Kidney, pancreas |
| 216a-5p | GCAACUGUGA | | UGAGAUUA | | |
| hsa-miR- | CAGUGCCUCGGCA | 29746 | GGGCUGCACUGCCG | 31788 | Lipid metabolism |
| 33b-3p | GUGCAGCCC | | AGGCACUG | | |
| hsa-miR- | GUGCAUUGCUGU | 29747 | GCAAUGCAACAGCA | 31789 | Lipid metabolism |
| 33b-5p | UGCAUUGC | | AUGCAC | | |
| hsa-miR- | ACUGGACUUGGA | 29748 | UUCUGCCUCCAAGU | 31790 | Lipid metabolism |
| 378b | GGCAGAA | | CCAGU | | |
| hsa-miR- | ACUGGACUUGGA | 29749 | CCACUCUUCUGACU | 31791 | Lipid metabolism |
| 378c | GUCAGAAGAGUG | | CCAAGUCCAGU | | |
| | G | | | | |
| hsa-miR- | ACUGGACUUGGA | 29750 | UUUCUGACUCCAAG | 31792 | Lipid metabolism |
| 378d | GUCAGAAA | | UCCAGU | | |
| hsa-miR- | ACUGGACUUGGA | 29751 | UCCUGACUCCAAGU | 31793 | Lipid metabolism |
| 378e | GUCAGGA | | CCAGU | | |
| hsa-miR- | ACUGGACUUGGA | 29752 | CUUCUGGCUCCAAG | 31794 | Lipid metabolism |
| 378f | GCCAGAAG | | UCCAGU | | |
| hsa-miR- | ACUGGGCUUGGA | 29753 | CUUCUGACUCCAAG | 31795 | Lipid metabolism |
| 378g | GUCAGAAG | | CCCAGU | | |
| hsa-miR- | ACUGGACUUGGU | 29754 | CCAUCUGACACCAA | 31796 | Lipid metabolism |
| 378h | GUCAGAUGG | | GUCCAGU | | |
| hsa-miR- | ACUGGACUAGGA | 29755 | CCUUCUGACUCCUA | 31797 | Lipid metabolism |
| 378i | GUCAGAAGG | | GUCCAGU | | |
| hsa-miR- | ACUGGAUUUGGA | 29756 | UUCUGGCUCCAAAU | 31798 | Lipid metabolism |
| 378 | GCCAGAA | | CCAGU | | |
| hsa-miR- | AGGAAUGUUCCU | 29757 | GGCAAAGAAGGAAC | 31799 | Lipid metabolism |
| 613 | UCUUUGCC | | AUUCCU | | |
| hsa-miR- | UCAGUGCAUGAC | 29758 | CCAAGUUCUGUCAU | 31800 | Liver |
| 152 | AGAACUUGG | | GCACUGA | | |
| hsa-miR- | UCACACCUGCCUC | 29759 | GGGGGGCGAGGCAG | 31801 | Liver (hepatocytes) |
| 1228-3p | GCCCCCC | | GUGUGA | | |
| hsa-miR- | GUGGGCGGGGGC | 29760 | CACACACCUGCCCC | 31802 | Liver (hepatocytes) |
| 1228-5p | AGGUGUGUG | | CGCCCAC | | |
| hsa-miR- | ACGCCCUUCCCCC | 29761 | UGAAGAAGGGGGGG | 31803 | Liver (hepatocytes) |
| 1249 | CCUUCUUCA | | AAGGGCGU | | |
| hsa-miR- | UUGCAUAUGUAG | 29762 | AUGGGACAUCCUAC | 31804 | Liver (hepatocytes) |
| 448 | GAUGUCCCAU | | AUAUGCAA | | |
| hsa-miR- | GUUUGCACGGGU | 29763 | AGACAAGGCCCACC | 31805 | Liver (hepatocytes) |
| 557 | GGGCCUUGUCU | | CGUGCAAAC | | |
| hsa-miR- | GACUAUAGAACU | 29764 | UGAGGGGGAAAGUU | 31806 | Liver (hepatocytes), |
| 625-3p | UUCCCCCUCA | | CUAUAGUC | | circulating (blood) |
| hsa-miR- | AGGGGGAAAGUU | 29765 | GGACUAUAGAACUU | 31807 | Liver (hepatocytes), |
| 625-5p | CUAUAGUCC | | UCCCCCU | | circulating (blood) |
| hsa-miR- | CUUUUUGCGGUC | 29766 | GCAAGCCCAGACCG | 31808 | Liver(hepatocytes) |
| 129-5p | UGGGCUUGC | | CAAAAAG | | |
| hsa-miR- | UCUUGUGUUCUC | 29767 | ACUGAUCUAGAGAA | 31809 | Liver(hepatocytes) |
| 581 | UAGAUCAGU | | CACAAGA | | |
| hsa-miR- | CCCAGUGUUCAGA | 29768 | GAACAGGUAGUCUG | 31810 | Liver, |
| 199a-5p | CUACCUGUUC | | AACACUGGG | | cardiomyocytes |
| hsa-miR- | ACAGUAGUCUGC | 29769 | UAACCAAUGUGCAG | 31811 | Liver, embryonic |
| 199a-3p | ACAUUGGUUA | | ACUACUGU | | body cells, |
| | | | | | cardiomyocytes |
| hsa-miR- | ACAGUAGUCUGC | 29770 | UAACCAAUGUGCAG | 31812 | Liver, osteoblast |
| 199b-3p | ACAUUGGUUA | | ACUACUGU | | |
| hsa-miR- | CCCAGUGUUUAG | 29771 | GAACAGAUAGUCUA | 31813 | Liver, osteoblast |
| 199b-5p | ACUAUCUGUUC | | AACACUGGG | | |
| hsa-miR- | UGGAGUGUGACA | 29772 | CAAACACCAUUGUC | 31814 | Liver/hepatocytes |
| 122-5p | AUGGUGUUUG | | ACACUCCA | | |
| hsa-miR- | UGCCCUAAAUGCC | 29773 | GCCAGAAGGGGCAU | 31815 | Lung |
| 18b-3p | CCUUCUGGC | | UUAGGGCA | | |
| hsa-miR- | UAAGGUGCAUCU | 29774 | CUAACUGCACUAGA | 31816 | Lung |
| 18b-5p | AGUGCAGUUAG | | UGCACCUUA | | |
| hsa-miR- | CUCCUAUAUGAU | 29775 | GAAGAAAGGCAUCA | 31817 | Lung |
| 337-3p | GCCUUUCUUC | | UAUAGGAG | | |
| hsa-miR- | GAACGGCUUCAU | 29776 | AACUCCUGUAUGAA | 31818 | Lung |
| 337-5p | ACAGGAGUU | | GCCGUUC | | |
| hsa-miR- | UGUGACUGGUUG | 29777 | CCCCUCUGGUCAAC | 31819 | Lung (epithelial) |
| 134 | ACCAGAGGGG | | CAGUCACA | | |
| hsa-miR- | ACUGCCCUAAGUG | 29778 | CCAGAAGGAGCACU | 31820 | Lung and |
| 18a-3p | CUCCUUCUGG | | UAGGGCAGU | | endothelial cells |
| hsa-miR- | UAAGGUGCAUCU | 29779 | CUAUCUGCACUAGA | 31821 | Lung and |
| 18a-5p | AGUGCAGAUAG | | UGCACCUUA | | endothelial cells |
| hsa-miR- | CAGUGCAAUGAU | 29780 | AUGCCCUUUCAUCA | 31822 | Lung, epidermal |
| 130b-3p | GAAAGGGCAU | | UUGCACUG | | cells( keratinocytes) |
| hsa-miR- | ACUCUUUCCCUGU | 29781 | GUAGUGCAACAGGG | 31823 | Lung, epidermal |
| 130b-5p | UGCACUAC | | AAAGAGU | | cells( keratinocytes) |
| hsa-miR- | UUUGGCAAUGGU | 29782 | AGUGUGAGUUCUAC | 31824 | Lung, immune cells |
| 182-5p | AGAACUCACACU | | CAUUGCCAAA | | |
| hsa-miR- | AGGGCCCCCCCUC | 29783 | ACAGGAUUGAGGGG | 31825 | Lung, liver, |
| 296-5p | AAUCCUGU | | GGGCCCU | | endothelial cells |
| hsa-miR- | CAGUGCAAUGUU | 29784 | AUGCCCUUUUAACA | 31826 | Lung, monocytes, |
| 130a-3p | AAAAGGGCAU | | UUGCACUG | | vascular endothelial |
| | | | | | cells |
| hsa-miR- | UUCACAUUGUGC | 29785 | GCAGACAGUAGCAC | 31827 | Lung, monocytes, |
| 130a-5p | UACUGUCUGC | | AAUGUGAA | | vascular endothelial |
| | | | | | cells |
| hsa-miR- | UCGGAUCCGUCUG | 29786 | AGCCAAGCUCAGAC | 31828 | Lung, placenta |
| 127-3p | AGCUUGGCU | | GGAUCCGA | | |
| hsa-miR- | CUGAAGCUCAGA | 29787 | AUCAGAGCCCUCUG | 31829 | Lung, |
| 127-5p | GGGCUCUGAU | | AGCUUCAG | | placenta(islet) |
| hsa-miR- | CCUCUUCCCCUUG | 29788 | CUGGAGAGACAAGG | 31830 | Lymphatic |
| 1236-3p | UCUCUCCAG | | GGAAGAGG | | endothelial cells |
| hsa-miR- | UGAGUGACAGGG | 29789 | UCCCCAUUUCCCCU | 31831 | Lymphatic |
| 1236-5p | GAAAUGGGGA | | GUCACUCA | | endothelial cells |
| hsa-miR- | UCAGGACACUUCU | 29790 | UCCAAGUUCAGAAG | 31832 | Lymphoblastic |
| 5000-3p | GAACUUGGA | | UGUCCUGA | | leukemia |
| hsa-miR- | CAGUUCAGAAGU | 29791 | ACUCAGGAACACUU | 31833 | Lymphoblastic |
| 5000-5p | GUUCCUGAGU | | CUGAACUG | | leukemia |
| hsa-miR- | UUUCCCUUUCCAU | 29792 | CUGCCAGGAUGGAA | 31834 | Lymphoblastic |
| 5006-3p | CCUGGCAG | | AGGGAAA | | leukemia |
| hsa-miR- | UUGCCAGGGCAG | 29793 | UUCCACCUCCUGCC | 31835 | Lymphoblastic |
| 5006-5p | GAGGUGGAA | | CUGGCAA | | leukemia |
| hsa-miR- | AGAGAUUGGUAG | 29794 | ACCUGAUUUCUACC | 31836 | Lymphoblastic |
| 5186 | AAAUCAGGU | | AAUCUCU | | leukemia |
| hsa-miR- | AAUCGGACCCAUU | 29795 | CUCCGGUUUAAAUG | 31837 | Lymphoblastic |
| 5188 | UAAACCGGAG | | GGUCCGAUU | | leukemia |
| hsa-miR- | UCUGGGCACAGGC | 29796 | CCUGUCCAUCCGCC | 31838 | Lymphoblastic |
| 5189 | GGAUGGACAGG | | UGUGCCCAGA | | leukemia |
| hsa-miR- | CCAGUGACUGAGC | 29797 | UGGCUCCAGCUCAG | 31839 | Lymphoblastic |
| 5190 | UGGAGCCA | | UCACUGG | | leukemia |
| hsa-miR- | AGGAUAGGAAGA | 29798 | AGCACUUCAUUCUU | 31840 | Lymphoblastic |
| 5191 | AUGAAGUGCU | | CCUAUCCU | | leukemia |
| hsa-miR- | AGGAGAGUGGAU | 29799 | ACCACCUGGAAUCC | 31841 | Lymphoblastic |
| 5192 | UCCAGGUGGU | | ACUCUCCU | | leukemia |
| hsa-miR- | UCCUCCUCUACCU | 29800 | ACUGGGAUGAGGUA | 31842 | Lymphoblastic |
| 5193 | CAUCCCAGU | | GAGGAGGA | | leukemia |
| hsa-miR- | UGAGGGGUUUGG | 29801 | CCAUCCCAUUCCAA | 31843 | Lymphoblastic |
| 5194 | AAUGGGAUGG | | ACCCCUCA | | leukemia |
| hsa-miR- | AUCCAGUUCUCUG | 29802 | AGCCCCCUCAGAGA | 31844 | Lymphoblastic |
| 5195-3p | AGGGGGCU | | ACUGGAU | | leukemia |
| hsa-miR- | AACCCCUAAGGCA | 29803 | CCAUCCAGUUGCCU | 31845 | Lymphoblastic |
| 5195-5p | ACUGGAUGG | | UAGGGGUU | | leukemia |
| hsa-miR- | UCAUCCUCGUCUC | 29804 | CUGGGAGGGAGACG | 31846 | Lymphoblastic |
| 5196-3p | CCUCCCAG | | AGGAUGA | | leukemia |
| hsa-miR- | AGGGAAGGGGAC | 29805 | CCCAACCCUCGUCC | 31847 | Lymphoblastic |
| 5196-5p | GAGGGUUGGG | | CCUUCCCU | | leukemia |
| hsa-miR- | AAGAAGAGACUG | 29806 | AUUCGAUGACUCAG | 31848 | Lymphoblastic |
| 5197-3p | AGUCAUCGAAU | | UCUCUUCUU | | leukemia |
| hsa-miR- | CAAUGGCACAAAC | 29807 | UCAAGAAUGAGUUU | 31849 | Lymphoblastic |
| 5197-5p | UCAUUCUUGA | | GUGCCAUUG | | leukemia |
| hsa-miR- | ACUGAAUCCUCUU | 29808 | CUGAGGAAAAGAGG | 31850 | Lymphoblastic |
| 5187-3p | UUCCUCAG | | AUUCAGU | | leukemia, skin |
| | | | | | (psoriasis) |
| hsa-miR- | UGGGAUGAGGGA | 29809 | UCCACUUCAAUCCC | 31851 | Lymphoblastic |
| 5187-5p | UUGAAGUGGA | | UCAUCCCA | | leukemia, skin |
| | | | | | (psoriasis) |
| hsa-miR- | CAGGCCAUAUUG | 29810 | UGAGGCAGCACAAU | 31852 | Lymphocyte, blood, |
| 15a-3p | UGCUGCCUCA | | AUGGCCUG | | hematopoietic |
| | | | | | tissues (spleen) |
| hsa-miR- | UAGCAGCACAUA | 29811 | CACAAACCAUUAUG | 31853 | Lymphocyte, blood, |
| 15a-5p | AUGGUUUGUG | | UGCUGCUA | | hematopoietic |
| | | | | | tissues (spleen) |
| hsa-miR- | CGAAUCAUUAUU | 29812 | UAGAGCAGCAAAUA | 31854 | Lymphocyte, blood, |
| 15b-3p | UGCUGCUCUA | | AUGAUUCG | | hematopoietic |
| | | | | | tissues (spleen) |
| hsa-miR- | UAGCAGCACAUCA | 29813 | UGUAAACCAUGAUG | 31855 | Lymphocyte, blood, |
| 15b-5p | UGGUUUACA | | UGCUGCUA | | hematopoietic |
| | | | | | tissues (spleen) |
| hsa-miR- | CCAAUAUUACUG | 29814 | UAAAGCAGCACAGU | 31856 | Lymphocyte, blood, |
| 16-2-3p | UGCUGCUUUA | | AAUAUUGG | | hematopoietic |
| | | | | | tissues (spleen) |
| hsa-miR- | CUAGGUAUGGUC | 29815 | GGAUCCCUGGGACC | 31857 | Lymphocytes |
| 331-5p | CCAGGGAUCC | | AUACCUAG | | |
| hsa-miR- | GUGUGUGGAAAU | 29816 | GCAGAAGCAUUUCC | 31858 | Macrophage |
| 147a | GCUUCUGC | | ACACAC | | |
| hsa-miR- | GUGUGCGGAAAU | 29817 | UAGCAGAAGCAUUU | 31859 | Macrophage |
| 147b | GCUUCUGCUA | | CCGCACAC | | |
| hsa-miR- | AGCAGCAUUGUA | 29818 | UGAUAGCCCUGUAC | 31860 | Many tissues and |
| 107 | CAGGGCUAUCA | | AAUGCUGCU | | brain |
| | | | | | hepatocytes/liver |
| hsa-miR- | AACUGGOCCUCAA | 29819 | AGCGGGACUUUGAG | 31861 | Many tissues/cells, |
| 193b-3p | AGUCCCGCU | | GGCCAGUU | | semen |
| hsa-miR- | CGGGGUUUUGAG | 29820 | UCAUCUCGCCCUCA | 31862 | Many tissues/cells, |
| 193b-5p | GGCGAGAUGA | | AAACCCCG | | semen |
| hsa-miR- | GCAGGGACAGCA | 29821 | GCACCCCUUUGCUG | 31863 | Melanocytes |
| 211-3p | AAGGGGUGC | | UCCCUGC | | |
| hsa-miR- | UUCCCUUUGUCAU | 29822 | AGGCGAAGGAUGAC | 31864 | Melanocytes |
| 211-5p | CCUUCGCCU | | AAAGGGAA | | |
| hsa-miR- | AUGGCCAAAACU | 29823 | AAAAUAACUGCAGU | 31865 | Melanoma |
| 548s | GCAGUUAUUUU | | UUUGGCCAU | | micrornaome |
| hsa-miR- | AAAAACCACAAU | 29824 | UGGUGCAAAAGUAA | 31866 | Melanoma |
| 548t-3p | UACUUUUGCACCA | | UUGUGGUUUUU | | micrornaome |
| hsa-miR- | CAAAAGUGAUCG | 29825 | CAAAAACCACGAUC | 31867 | Melanoma |
| 548t-5p | UGGUUUUUG | | ACUUUUG | | micrornaome |
| hsa-miR- | CAAAGACUGCAA | 29826 | CGCAAAAGUAAUUG | 31868 | Melanoma |
| 548u | UUACUUUUGCG | | CAGUCUUUG | | micrornaome |
| hsa-miR- | AAAAGUAACUGC | 29827 | AGGCAAAAACCGCA | 31869 | Melanoma |
| 548w | GGUUUUUGCCU | | GUUACUUUU | | micrornaome |
| hsa-miR- | UGCCUGGAACAU | 29828 | AGUCCCUACUAUGU | 31870 | Melanoma |
| 3116 | AGUAGGGACU | | UCCAGGCA | | miRNAome |
| hsa-miR- | AUAGGACUCAUA | 29829 | CUGGCACUAUAUGA | 31871 | Melanoma |
| 3117-3p | UAGUGCCAG | | GUCCUAU | | miRNAome |
| hsa-miR- | AGACACUAUACG | 29830 | AUAUGACUCGUAUA | 31872 | Melanoma |
| 3117-5p | AGUCAUAU | | GUGUCU | | miRNAome |
| hsa-miR- | UGUGACUGCAUU | 29831 | AGAAUUUUCAUAAU | 31873 | Melanoma |
| 3118 | AUGAAAAUUCU | | GCAGUCACA | | miRNAome |
| hsa-miR- | UGGCUUUUAACU | 29832 | GCCAUCAAAGUUAA | 31874 | Melanoma |
| 3119 | UUGAUGGC | | AAGCCA | | miRNAome |
| hsa-miR- | CACAGCAAGUGU | 29833 | UGCCUGUCUACACU | 31875 | Melanoma |
| 3120-3p | AGACAGGCA | | UGCUGUG | | miRNAome |
| hsa-miR- | CCUGUCUGUGCCU | 29834 | UGUACAGCAGGCAC | 31876 | Melanoma |
| 3120-5p | GCUGUACA | | AGACAGG | | miRNAome |
| hsa-miR- | UAAAUAGAGUAG | 29835 | UGUCCUUUGCCUAC | 31877 | Melanoma |
| 3121-3p | GCAAAGGACA | | UCUAUUUA | | miRNAome |
| hsa-miR- | UCCUUUGCCUAUU | 29836 | CUUAAAUAGAAUAG | 31878 | Melanoma |
| 3121-5p | CUAUUUAAG | | GCAAAGGA | | miRNAome |
| hsa-miR- | GUUGGGACAAGA | 29837 | AAGACCGUCCUCUU | 31879 | Melanoma |
| 3122 | GGACGGUCUU | | GUCCCAAC | | miRNAome |
| hsa-miR- | CAGAGAAUUGUU | 29838 | GAUUAAACAAUUCU | 31880 | Melanoma |
| 3123 | UAAUC | | CUG | | miRNAome |
| hsa-miR- | UAGAGGAAGCUG | 29839 | UCUCUCCACAGCUU | 31881 | Melanoma |
| 3125 | UGGAGAGA | | CCUCUA | | miRNAome |
| hsa-miR- | UCCCCUUCUGCAG | 29840 | CCAGCAGGCCUGCA | 31882 | Melanoma |
| 3127-3p | GCCUGCUGG | | GAAGGGGA | | miRNAome |
| hsa-miR- | AUCAGGGCUUGU | 29841 | CUUCCCAUUCCACA | 31883 | Melanoma |
| 3127-5p | GGAAUGGGAAG | | AGCCCUGAU | | miRNAome |
| hsa-miR- | UCUGGCAAGUAA | 29842 | AUGAGAGUUUUUUA | 31884 | Melanoma |
| 3128 | AAAACUCUCAU | | CUUGCCAGA | | miRNAome |
| hsa-miR- | UCGAGGACUGGU | 29843 | AAGGCCCUUCCACC | 31885 | Melanoma |
| 3131 | GGAAGGGCCUU | | AGUCCUCGA | | miRNAome |
| hsa-miR- | UGGGUAGAGAAG | 29844 | UCCUCUGAGCUCCU | 31886 | Melanoma |
| 3132 | GAGCUCAGAGGA | | UCUCUACCCA | | miRNAome |
| hsa-miR- | UAAAGAACUCUU | 29845 | AUUGGGUUUUAAGA | 31887 | Melanoma |
| 3133 | AAAACCCAAU | | GUUCUUUA | | miRNAome |
| hsa-miR- | UGAUGGAUAAAA | 29846 | AAUAUGUAGUCUUU | 31888 | Melanoma |
| 3134 | GACUACAUAUU | | UAUCCAUCA | | miRNAome |
| hsa-miR- | UGCCUAGGCUGA | 29847 | CACUGCAGUCUCAG | 31889 | Melanoma |
| 3135a | GACUGCAGUG | | CCUAGGCA | | miRNAome |
| hsa-miR- | UGGCCCAACCUAU | 29848 | ACUAACUGAAUAGG | 31890 | Melanoma |
| 3136-3p | UCAGUUAGU | | UUGGGCCA | | miRNAome |
| hsa-miR- | CUGACUGAAUAG | 29849 | AAUGACCCUACCUA | 31891 | Melanoma |
| 3136-5p | GUAGGGUCAUU | | UUCAGUCAG | | miRNAome |
| hsa-miR- | UCUGUAGCCUGG | 29850 | ACCCCAUUGCUCCC | 31892 | Melanoma |
| 3137 | GAGCAAUGGGGU | | AGGCUACAGA | | miRNAome |
| hsa-miR- | UAGGAGCUCAAC | 29851 | AACAGGCAUCUGUU | 31893 | Melanoma |
| 3139 | AGAUGCCUGUU | | GAGCUCCUA | | miRNAome |
| hsa-miR- | GAGGGCGGGUGG | 29852 | UCCUCCUCCACCCG | 31894 | Melanoma |
| 3141 | AGGAGGA | | CCCUC | | miRNAome |
| hsa-miR- | AUAACAUUGUAA | 29853 | CGAAAGAAGCGCUU | 31895 | Melanoma |
| 3143 | AGCGCUUCUUUCG | | UACAAUGUUAU | | miRNAome |
| hsa-miR- | AGAUAUUUUGAG | 29854 | CAAUUCCAAACACU | 31896 | Melanoma |
| 3145-3p | UGUUUGGAAUUG | | CAAAAUAUCU | | miRNAome |
| hsa-miR- | AACUCCAAACACU | 29855 | UGAGUUUUGAGUGU | 31897 | Melanoma |
| 3145-5p | CAAAACUCA | | UUGGAGUU | | miRNAome |
| hsa-miR- | CAUGCUAGGAUA | 29856 | CCAUUCUUUCUAUC | 31898 | Melanoma |
| 3146 | GAAAGAAUGG | | CUAGCAUG | | miRNAome |
| hsa-miR- | GGUUGGGCAGUG | 29857 | UCACACCCUCCUCA | 31899 | Melanoma |
| 3147 | AGGAGGGUGUGA | | CUGCCCAACC | | miRNAome |
| hsa-miR- | UGGAAAAAACUG | 29858 | AAGCACACACCAGU | 31900 | Melanoma |
| 3148 | GUGUGUGCUU | | UUUUUCCA | | miRNAome |
| hsa-miR- | CUGGGGAGAUCC | 29859 | CCAACCUCGAGGAU | 31901 | Melanoma |
| 3150a-3p | UCGAGGUUGG | | CUCCCCAG | | miRNAome |
| hsa-miR- | CAACCUCGACGAU | 29860 | GCUGAGGAGAUCGU | 31902 | Melanoma |
| 3150a-5p | CUCCUCAGC | | CGAGGUUG | | miRNAome |
| hsa-miR- | UGAGGAGAUCGU | 29861 | CCAACCUCGACGAU | 31903 | Melanoma |
| 3150b-3p | CGAGGUUGG | | CUCCUCA | | miRNAome |
| hsa-miR- | CAACCUCGAGGAU | 29862 | GCUGGGGAGAUCCU | 31904 | Melanoma |
| 3150b-5p | CUCCCCAGC | | CGAGGUUG | | miRNAome |
| hsa-miR- | GGUGGGGCAAUG | 29863 | ACCUGAUCCCAUUG | 31905 | Melanoma |
| 3151 | GGAUCAGGU | | CCCCACC | | miRNAome |
| hsa-miR- | GGGGAAAGCGAG | 29864 | AAAUGUCCCUACUC | 31906 | Melanoma |
| 3153 | UAGGGACAUUU | | GCUUUCCCC | | miRNAome |
| hsa-miR- | CAGAAGGGGAGU | 29865 | UCUGCUCCCAACUC | 31907 | Melanoma |
| 3154 | UGGGAGCAGA | | CCCUUCUG | | miRNAome |
| hsa-miR- | CCAGGCUCUGCAG | 29866 | AGUUCCCACUGCAG | 31908 | Melanoma |
| 3155a | UGGGAACU | | AGCCUGG | | miRNAome |
| hsa-miR- | CUCCCACUUCCAG | 29867 | AGAAAGAUCUGGAA | 31909 | Melanoma |
| 3156-3p | AUCUUUCU | | GUGGGAG | | miRNAome |
| hsa-miR- | AAAGAUCUGGAA | 29868 | UGUCUCCCACUUCC | 31910 | Melanoma |
| 3156-5p | GUGGGAGACA | | AGAUCUUU | | miRNAome |
| hsa-miR- | CUGCCCUAGUCUA | 29869 | AGCUUCAGCUAGAC | 31911 | Melanoma |
| 3157-3p | GCUGAAGCU | | UAGGGCAG | | miRNAome |
| hsa-miR- | UUCAGCCAGGCUA | 29870 | AGACUGCACUAGCC | 31912 | Melanoma |
| 3157-5p | GUGCAGUCU | | UGGCUGAA | | miRNAome |
| hsa-miR- | UAGGAUUACAAG | 29871 | GUGGCCGACACUUG | 31913 | Melanoma |
| 3159 | UGUCGGCCAC | | UAAUCCUA | | miRNAome |
| hsa-miR- | AGAGCUGAGACU | 29872 | UGGGCUUUCUAGUC | 31914 | Melanoma |
| 3160-3p | AGAAAGCCCA | | UCAGCUCU | | miRNAome |
| hsa-miR- | GGCUUUCUAGUC | 29873 | GGAGAGCUGAGACU | 31915 | Melanoma |
| 3160-5p | UCAGCUCUCC | | AGAAAGCC | | miRNAome |
| hsa-miR- | CUGAUAAGAACA | 29874 | AUCUGGGCCUCUGU | 31916 | Melanoma |
| 3161 | GAGGCCCAGAU | | UCUUAUCAG | | miRNAome |
| hsa-miR- | UCCCUACCCCUCC | 29875 | UGGGGAGUGGAGGG | 31917 | Melanoma |
| 3162-3p | ACUCCCCA | | GUAGGGA | | miRNAome |
| hsa-miR- | UUAGGGAGUAGA | 29876 | CUCCCCACCCUUCU | 31918 | Melanoma |
| 3162-5p | AGGGUGGGGAG | | ACUCCCUAA | | miRNAome |
| hsa-miR- | UAUAAAAUGAGG | 29877 | GUCUUACUGCCCUC | 31919 | Melanoma |
| 3163 | GCAGUAAGAC | | AUUUUAUA | | miRNAome |
| hsa-miR- | UGUGACUUUAAG | 29878 | CGCCAUUUCCCUUA | 31920 | Melanoma |
| 3164 | GGAAAUGGCG | | AAGUCACA | | miRNAome |
| hsa-miR- | AGGUGGAUGCAA | 29879 | UGAGGUCACAUUGC | 31921 | Melanoma |
| 3165 | UGUGACCUCA | | AUCCACCU | | miRNAome |
| hsa-miR- | CGCAGACAAUGCC | 29880 | UAGGCCAGUAGGCA | 31922 | Melanoma |
| 3166 | UACUGGCCUA | | UUGUCUGCG | | miRNAome |
| hsa-miR- | GAGUUCUACAGU | 29881 | GUCUGACUGUAGAA | 31923 | Melanoma |
| 3168 | CAGAC | | CUC | | miRNAome |
| hsa-miR- | UAGGACUGUGCU | 29882 | CUAUGUGCCAAGCA | 31924 | Melanoma |
| 3169 | UGGCACAUAG | | CAGUCCUA | | miRNAome |
| hsa-miR- | CUGGGGUUCUGA | 29883 | ACUGUCUGUCUCAG | 31925 | Melanoma |
| 3170 | GACAGACAGU | | AACCCCAG | | miRNAome |
| hsa-miR- | AAAGGAGGAAAU | 29884 | UGGCCUGCCUAUUU | 31926 | Melanoma |
| 3173-3p | AGGCAGGCCA | | CCUCCUUU | | miRNAome |
| hsa-miR- | UGCCCUGCCUGUU | 29885 | AAAGGAGAAAACAG | 31927 | Melanoma |
| 3173-5p | UUCUCCUUU | | GCAGGGCA | | miRNAome |
| hsa-miR- | UAGUGAGUUAGA | 29886 | GGCUCUGCAUCUCU | 31928 | Melanoma |
| 3174 | GAUGCAGAGCC | | AACUCACUA | | miRNAome |
| hsa-miR- | ACUGGCCUGGGAC | 29887 | CCGGUAGUCCCAGG | 31929 | Melanoma |
| 3176 | UACCGG | | CCAGU | | miRNAome |
| hsa-miR- | UGCACGGCACUGG | 29888 | ACGUGUCCCCAGUG | 31930 | Melanoma |
| 3177-3p | GGACACGU | | CCGUGCA | | miRNAome |
| hsa-miR- | UGUGUACACACG | 29889 | AGCGCCUGGCACGU | 31931 | Melanoma |
| 3177-5p | UGCCAGGCGCU | | GUGUACACA | | miRNAome |
| hsa-miR- | GGGGCGCGGCCGG | 29890 | CGAUCCGGCCGCGC | 31932 | Melanoma |
| 3178 | AUCG | | CCC | | miRNAome |
| hsa-miR- | AGAAGGGGUGAA | 29891 | ACGUUUAAAUUUCA | 31933 | Melanoma |
| 3179 | AUUUAAACGU | | CCCCUUCU | | miRNAome |
| hsa-miR- | AUCGGGCCCUCGG | 29892 | CCGGCGCCGAGGGC | 31934 | Melanoma |
| 3181 | CGCCGG | | CCGAU | | miRNAome |
| hsa-miR- | GCUUCUGUAGUG | 29893 | GACUACACUACAGA | 31935 | Melanoma |
| 3182 | UAGUC | | AGC | | miRNAome |
| hsa-miR- | GCCUCUCUCGGAG | 29894 | UCCGAGCGACUCCG | 31936 | Melanoma |
| 3183 | UCGCUCGGA | | AGAGAGGC | | miRNAome |
| hsa-miR- | AAAGUCUCGCUCU | 29895 | UGAGGGGCAGAGAG | 31937 | Melanoma |
| 3184-3p | CUGCCCCUCA | | CGAGACUUU | | miRNAome |
| hsa-miR- | UGAGGGGCCUCA | 29896 | AAAAGCUCGGUCUG | 31938 | Melanoma |
| 3184-5p | GACCGAGCUUUU | | AGGCCCCUCA | | miRNAome |
| hsa-miR- | AGAAGAAGGCGG | 29897 | CCGCAGACCGACCG | 31939 | Melanoma |
| 3185 | UCGGUCUGCGG | | CCUUCUUCU | | miRNAome |
| hsa-miR- | UUGGCCAUGGGG | 29898 | CCGCGCAGCCCCAU | 31940 | Melanoma |
| 3187-3p | CUGCGCGG | | GGCCAA | | miRNAome |
| hsa-miR- | CCUGGGCAGCGUG | 29899 | CCUUCAGCCACACG | 31941 | Melanoma |
| 3187-5p | UGGCUGAAGG | | CUGCCCAGG | | miRNAome |
| hsa-miR- | AGAGGCUUUGUG | 29900 | CCCCGUAUCCGCAC | 31942 | Melanoma |
| 3188 | CGGAUACGGGG | | AAAGCCUCU | | miRNAome |
| hsa-miR- | CCCUUGGGUCUGA | 29901 | CUACCCCAUCAGAC | 31943 | Melanoma |
| 3189-3p | UGGGGUAG | | CCAAGGG | | miRNAome |
| hsa-miR- | UGCCCCAUCUGUG | 29902 | UCCUACCCAGGGCA | 31944 | Melanoma |
| 3189-5p | CCCUGGGUAGGA | | CAGAUGGGGCA | | miRNAome |
| hsa-miR- | UGUGGAAGGUAG | 29903 | UCUCUGGCCGUCUA | 31945 | Melanoma |
| 3190-3p | ACGGCCAGAGA | | CCUUCCACA | | miRNAome |
| hsa-miR- | UCUGGCCAGCUAC | 29904 | UGGGGACGUAGCUG | 31946 | Melanoma |
| 3190-5p | GUCCCCA | | GCCAGA | | miRNAome |
| hsa-miR- | UGGGGACGUAGC | 29905 | CUGUCUGGCCAGCU | 31947 | Melanoma |
| 3191-3p | UGGCCAGACAG | | ACGUCCCCA | | miRNAome |
| hsa-miR- | CUCUCUGGCCGUC | 29906 | UGGAAGGUAGACGG | 31948 | Melanoma |
| 3191-5p | UACCUUCCA | | CCAGAGAG | | miRNAome |
| hsa-miR- | UCUGGGAGGUUG | 29907 | UUCCACUGCUACAA | 31949 | Melanoma |
| 3192 | UAGCAGUGGAA | | CCUCCCAGA | | miRNAome |
| hsa-miR- | UCCUGCGUAGGA | 29908 | ACUCCUCAGAUCCU | 31950 | Melanoma |
| 3193 | UCUGAGGAGU | | ACGCAGGA | | miRNAome |
| hsa-miR- | AGCUCUGCUGCUC | 29909 | ACUGCCAGUGAGCA | 31951 | Melanoma |
| 3194-3p | ACUGGCAGU | | GCAGAGCU | | miRNAome |
| hsa-miR- | GGCCAGCCACCAG | 29910 | CAGCCCUCCUGGUG | 31952 | Melanoma |
| 3194-5p | GAGGGCUG | | GCUGGCC | | miRNAome |
| hsa-miR- | CGCGCCGGGCCCG | 29911 | AACCCGGGCCCGGC | 31953 | Melanoma |
| 3195 | GGUU | | GCG | | miRNAome |
| hsa-miR- | GGAGGCGCAGGO | 29912 | CGCCUUUCCGAGCC | 31954 | Melanoma |
| 3197 | UCGGAAAGGCG | | UGCGCCUCC | | miRNAome |
| hsa-miR- | GUGGAGUCCUGG | 29913 | UCUCCAUUCCCCAG | 31955 | Melanoma |
| 3198 | GGAAUGGAGA | | GACUCCAC | | miRNAome |
| hsa-miR- | AGGGACUGCCUU | 29914 | AACUUUCUCCUAAG | 31956 | Melanoma |
| 3199 | AGGAGAAAGUU | | GCAGUCCCU | | miRNAome |
| hsa-miR- | GGGAUAUGAAGA | 29915 | AUUUUUCUUCAUAU | 31957 | Melanoma |
| 3201 | AAAAU | | CCC | | miRNAome, |
| hsa-miR- | UGGAAGGGAGAA | 29916 | AUUAAAGCUCUUCU | 31958 | Melanoma |
| 3202 | GAGCUUUAAU | | CCCUUCCA | | miRNAome, |
| | | | | | epithelial cell |
| | | | | | BEAS2B |
| hsa-miR- | ACUUUCCUCACUC | 29917 | ACUUCACGGGAGUG | 31959 | Melanoma |
| 3124-3p | CCGUGAAGU | | AGGAAAGU | | miRNAome, ovary |
| hsa-miR- | UUCGCGGGCGAA | 29918 | GACUUUGCCUUCGC | 31960 | Melanoma |
| 3124-5p | GGCAAAGUC | | CCGCGAA | | miRNAome, ovary |
| hsa-miR- | CAUCUGGCAUCCG | 29919 | UCUGUGUGACGGAU | 31961 | Melanoma |
| 3126-3p | UCACACAGA | | GCCAGAUG | | miRNAome, ovary |
| hsa-miR- | UGAGGGACAGAU | 29920 | UGCUUCUGGCAUCU | 31962 | Melanoma |
| 3126-5p | GCCAGAAGCA | | GUCCCUCA | | miRNAome, ovary |
| hsa-miR- | AAACUAAUCUCU | 29921 | GCAGCAGUGUAGAG | 31963 | Melanoma |
| 3129-3p | ACACUGCUGC | | AUUAGUUU | | miRNAome, ovary |
| hsa-miR- | GCAGUAGUGUAG | 29922 | AAACCAAUCUCUAC | 31964 | Melanoma |
| 3129-5p | AGAUUGGUUU | | ACUACUGC | | miRNAome, ovary |
| hsa-miR- | GCUGCACCGGAGA | 29923 | UUACCCAGUCUCCG | 31965 | Melanoma |
| 3130-3p | CUGGGUAA | | GUGCAGC | | miRNAome, ovary |
| hsa-miR- | UACCCAGUCUCCG | 29924 | GGCUGCACCGGAGA | 31966 | Melanoma |
| 3130-5p | GUGCAGCC | | CUGGGUA | | miRNAome, ovary |
| hsa-miR- | UGUGGACAGUGA | 29925 | ACUCCCUCUACCUC | 31967 | Melanoma |
| 3138 | GGUAGAGGGAGU | | ACUGUCCACA | | miRNAome, ovary |
| hsa-miR- | AGCUUUUGGGAA | 29926 | ACUACCUGAAUUCC | 31968 | Melanoma |
| 3140-3p | UUCAGGUAGU | | CAAAAGCU | | miRNAome, ovary |
| hsa-miR- | ACCUGAAUUACCA | 29927 | AAAGCUUUUGGUAA | 31969 | Melanoma |
| 3140-5p | AAAGCUUU | | UUCAGGU | | miRNAome, ovary |
| hsa-miR- | AUAUACCUGUUC | 29928 | UAAAGAGACCGAAC | 31970 | Melanoma |
| 3144-3p | GGUCUCUUUA | | AGGUAUAU | | miRNAome, ovary |
| hsa-miR- | AGGGGACCAAAG | 29929 | CUAUAUAUCUCUUU | 31971 | Melanoma |
| 3144-5p | AGAUAUAUAG | | GGUCCCCU | | miRNAome, ovary |
| hsa-miR- | UUUGUAUGGAUA | 29930 | AUACACACACAUAU | 31972 | Melanoma |
| 3149 | UGUGUGUGUAU | | CCAUACAAA | | miRNAome, ovary |
| hsa-miR- | UGUGUUAGAAUA | 29931 | UUAUUGCCCCUAUU | 31973 | Melanoma |
| 3152-3p | GGGGCAAUAA | | CUAACACA | | miRNAome, ovary |
| hsa-miR- | AUUGCCUCUGUUC | 29932 | CUUGUGUUAGAACA | 31974 | Melanoma |
| 3152-5p | UAACACAAG | | GAGGCAAU | | miRNAome, ovary |
| hsa-miR- | AAGGGCUUCCUCU | 29933 | GUCCUGCAGAGAGG | 31975 | Melanoma |
| 3158-3p | CUGCAGGAC | | AAGCCCUU | | miRNAome, ovary |
| hsa-miR- | CCUGCAGAGAGG | 29934 | GAAGGGCUUCCUCU | 31976 | Melanoma |
| 3158-5p | AAGCCCUUC | | CUGCAGG | | miRNAome, ovary |
| hsa-miR- | AGGAUUUCAGAA | 29935 | ACACCAGUAUUUCU | 31977 | Melanoma |
| 3167 | AUACUGGUGU | | GAAAUCCU | | miRNAome, ovary |
| hsa-miR- | AGAUGUAUGGAA | 29936 | GAUAUAUACAGAUU | 31978 | Melanoma |
| 3171 | UCUGUAUAUAUC | | CCAUACAUCU | | miRNAome, ovary |
| hsa-miR- | CGGGGAGAGAAC | 29937 | ACGUCACUGCGUUC | 31979 | Melanoma |
| 3175 | GCAGUGACGU | | UCUCCCCG | | miRNAome, ovary |
| hsa-miR- | UGGGGCGGAGCU | 29938 | CUCCGGAAGCUCCG | 31980 | Melanoma |
| 3180 | UCCGGAG | | CCCCA | | miRNAome, ovary |
| hsa-miR- | UCACGCGGAGAG | 29939 | CAAAGCCAUCUCUC | 31981 | Melanoma |
| 3186-3p | AUGGCUUUG | | CGCGUGA | | miRNAome, ovary |
| hsa-miR- | CAGGCGUCUGUCU | 29940 | AAGCCACGUAGACA | 31982 | Melanoma |
| 3186-5p | ACGUGGCUU | | GACGCCUG | | miRNAome, ovary |
| hsa-miR- | CACCUUGCGCUAC | 29941 | CAGACCUGAGUAGC | 31983 | Melanoma |
| 3200-3p | UCAGGUCUG | | GCAAGGUG | | miRNAome, ovary |
| hsa-miR- | AAUCUGAGAAGG | 29942 | ACCUUGUGOGCCUU | 31984 | Melanoma |
| 3200-5p | CGCACAAGGU | | CUCAGAUU | | miRNAome, ovary |
| hsa-miR- | AAGGCCUUUCUG | 29943 | UCUGAAGGUUCAGA | 31985 | Melanoma |
| 3142 | AACCUUCAGA | | AAGGCCUU | | miRNAome; |
| | | | | | immune cells |
| hsa-miR- | GUGACAUCACAU | 29944 | GCUGCCGUAUAUGU | 31986 | Mesenchymal stem |
| 489 | AUACGGCAGC | | GAUGUCAC | | cells |
| hsa-miR- | UCAGAACAAAUG | 29945 | UCUGGGAACCGGCA | 31987 | Mesothelial cells |
| 589-3p | CCGGUUCCCAGA | | UUUGUUCUGA | | |
| hsa-miR- | UGAGAACCACGUC | 29946 | CUCAGAGCAGACGU | 31988 | Mesothelial cells |
| 589-5p | UGCUCUGAG | | GGUUCUCA | | |
| hsa-miR- | UCAUAUUGCUUC | 29947 | AGAAAGAAGCAAUA | 31989 | Monocytes |
| 1279 | UUUCU | | UGA | | |
| hsa-miR- | UGUGACAGAUUG | 29948 | UUUCAGUUAUCAAU | 31990 | Monocytes |
| 542-3p | AUAACUGAAA | | CUGUCACA | | |
| hsa-miR- | UGGAGUCCAGGA | 29949 | AAAAUGCAGAUUCC | 31991 | Multiple cell types |
| 1289 | AUCUGCAUUUU | | UGGACUCCA | | |
| hsa-miR- | AAGCCCUUACCCC | 29950 | AUACUUUUUGGGGU | 31992 | Multiple cell types |
| 129-1-3p | AAAAAGUAU | | AAGGGCUU | | |
| hsa-miR- | AAGCCCUUACCCC | 29951 | AUGCUUUUUGGGGU | 31993 | Multiple cell types |
| 129-2-3p | AAAAAGCAU | | AAGGGCUU | | |
| hsa-miR- | UGGAAUGUAAGG | 29952 | CCACACACUUCCUU | 31994 | Muscle (cardiac and |
| 206 | AAGUGUGUGG | | ACAUUCCA | | skeletal) |
| hsa-miR- | CUUAUCAGAUUG | 29953 | AAUUACAAUACAAU | 31995 | Muscle (myoblasts) |
| 374a-3p | UAUUGUAAUU | | CUGAUAAG | | |
| hsa-miR- | UUAUAAUACAAC | 29954 | CACUUAUCAGGUUG | 31996 | Muscle (myoblasts) |
| 374a-5p | CUGAUAAGUG | | UAUUAUAA | | |
| hsa-miR- | CUUAGCAGGUUG | 29955 | AAUGAUAAUACAAC | 31997 | Muscle (myoblasts) |
| 374b-3p | UAUUAUCAUU | | CUGCUAAG | | |
| hsa-miR- | AUAUAAUACAAC | 29956 | CACUUAGCAGGUUG | 31998 | Muscle (myoblasts) |
| 374b-5p | CUGCUAAGUG | | UAUUAUAU | | |
| hsa-miR- | CACUUAGCAGGU | 29957 | AUAUAAUACAACCU | 31999 | Muscle (myoblasts) |
| 374c-3p | UGUAUUAUAU | | GCUAAGUG | | |
| hsa-miR- | AUAAUACAACCU | 29958 | AGCACUUAGCAGGU | 32000 | Muscle (myoblasts) |
| 374c-5p | GCUAAGUGCU | | UGUAUUAU | | |
| hsa-miR- | UUUGGUCCCCUUC | 29959 | CAGCUGGUUGAAGG | 32001 | Muscle, heart, |
| 133a | AACCAGCUG | | GGACCAAA | | epithelial cells |
| | | | | | (lung) |
| hsa-miR- | UUUGGUCCCCUUC | 29960 | UAGCUGGUUGAAGG | 32002 | Muscle, heart, |
| 133b | AACCAGCUA | | GGACCAAA | | epithelial cells |
| | | | | | (lung) |
| hsa-miR- | UGUAAACAUCCU | 29961 | CUUCCAGUCAAGGA | 32003 | Myeloid cell and |
| 30e-5p | UGACUGGAAG | | UGUUUACA | | glia cells |
| hsa-miR- | UGUCAGUUUGUC | 29962 | UGGGGUAUUUGACA | 32004 | Myeloid cells |
| 223-3p | AAAUACCCCA | | AACUGACA | | |
| hsa-miR- | CGUGUAUUUGAC | 29963 | AACUCAGCUUGUCA | 32005 | Myeloid cells |
| 223-5p | AAGCUGAGUU | | AAUACACG | | |
| hsa-miR- | UUCACAGUGGCU | 29964 | GCGGAACUUAGCCA | 32006 | Myeloid cells |
| 27a-3p | AAGUUCCGC | | CUGUGAA | | |
| hsa-miR- | AGGGCUUAGCUG | 29965 | UGCUCACAAGCAGC | 32007 | Myeloid cells |
| 27a-5p | CUUGUGAGCA | | UAAGCCCU | | |
| hsa-miR- | AUCACAUUGCCAG | 29966 | GGUAAUCCCUGGCA | 32008 | Myeloid cells and |
| 23b-3p | GGAUUACC | | AUGUGAU | | blood |
| hsa-miR- | UGGGUUCCUGGC | 29967 | AAAUCAGCAUGCCA | 32009 | Myeloid cells and |
| 23b-5p | AUGCUGAUUU | | GGAACCCA | | blood |
| hsa-miR- | CUUUCAGUCGGA | 29968 | GCUGUAAACAUCCG | 32010 | Myeloid cells and |
| 30e-3p | UGUUUACAGC | | ACUGAAAG | | glia cells |
| hsa-miR- | UGCCUACUGAGCU | 29969 | ACUGAUAUCAGCUC | 32011 | Myeloid cells and |
| 24-1-5p | GAUAUCAGU | | AGUAGGCA | | lung |
| hsa-miR- | UGCCUACUGAGCU | 29970 | CUGUGUUUCAGCUC | 32012 | Myeloid cells and |
| 24-2-5p | GAAACACAG | | AGUAGGCA | | lung |
| hsa-miR- | UGGCUCAGUUCA | 29971 | CUGUUCCUGCUGAA | 32013 | Myeloid cells and |
| 24-3p | GCAGGAACAG | | CUGAGCCA | | lung |
| hsa-miR- | UUCACAGUGGCU | 29972 | GCAGAACUUAGCCA | 32014 | Myeloid cells and |
| 27b-3p | AAGUUCUGC | | CUGUGAA | | vascular endothelial |
| | | | | | cells |
| hsa-miR- | AGAGCUUAGCUG | 29973 | GUUCACCAAUCAGC | 32015 | Myeloid cells and |
| 27b-5p | AUUGGUGAAC | | UAAGCUCU | | vascular endothelial |
| | | | | | cells |
| hsa-miR- | UGUAGUGUUUCC | 29974 | UCCAUAAAGUAGGA | 32016 | Myeloid cells, |
| 142-3p | UACUUUAUGGA | | AACACUACA | | hematopoiesis, |
| | | | | | APC cells |
| hsa-miR- | CAUAAAGUAGAA | 29975 | AGUAGUGCUUUCUA | 32017 | Myeloid cells, |
| 142-5p | AGCACUACU | | CUUUAUG | | hematopoiesis, |
| | | | | | APC cells |
| hsa-miR- | UGAAGGUCUACU | 29976 | CCUGGCACACAGUA | 32018 | Myeloid cells, |
| 493-3p | GUGUGCCAGG | | GACCUUCA | | pancreas (islet) |
| hsa-miR- | UUGUACAUGGUA | 29977 | AAUGAAAGCCUACC | 32019 | Myeloid cells, |
| 493-5p | GGCUUUCAUU | | AUGUACAA | | pancreas (islet) |
| hsa-miR- | CUGGAUGGCUCCU | 29978 | AGACAUGGAGGAGC | 32020 | Myoblast |
| 432-3p | CCAUGUCU | | CAUCCAG | | |
| hsa-miR- | UCUUGGAGUAGG | 29979 | CCACCCAAUGACCU | 32021 | Myoblast |
| 432-5p | UCAUUGGGUGG | | ACUCCAAGA | | |
| hsa-miR- | CUCAUCUGCAAAG | 29980 | CACUUACUUCUUUG | 32022 | Myoblast |
| 452-3p | AAGUAAGUG | | CAGAUGAG | | |
| hsa-miR- | AACUGUUUGCAG | 29981 | UCAGUUUCCUCUGC | 32023 | Myoblast |
| 452-5p | AGGAAACUGA | | AAACAGUU | | |
| hsa-miR- | CUUGGUUCAGGG | 29982 | UGGGGACCCUCCCU | 32024 | Myoblast |
| 659-3p | AGGGUCCCCA | | GAACCAAG | | |
| hsa-miR- | AGGACCUUCCCUG | 29983 | UCCUUGGUUCAGGG | 32025 | Myoblast |
| 659-5p | AACCAAGGA | | AAGGUCCU | | |
| hsa-miR- | ACCUCCUGUGUGC | 29984 | UAAUCCAUGCACAC | 32026 | Myoblast |
| 660-3p | AUGGAUUA | | AGGAGGU | | |
| hsa-miR- | UACCCAUUGCAUA | 29985 | CAACUCCGAUAUGC | 32027 | Myoblast |
| 660-5p | UCGGAGUUG | | AAUGGGUA | | |
| hsa-miR- | AAAGCUGGGUUG | 29986 | CCUUCUCAACCCAG | 32028 | Neural cells |
| 320e | AGAAGG | | CUUU | | |
| hsa-miR- | GGUAUCCGUUUG | 29987 | ACCAUCCCCAAACG | 32029 | Neuroblastoma |
| 3713 | GGGAUGGU | | GAUACC | | |
| hsa-miR- | GAAGGCAGCAGU | 29988 | ACAGGGGAGCACUG | 32030 | Neuroblastoma |
| 3714 | GCUCCCCUGU | | CUGCCUUC | | |
| hsa-miR- | UCAGUGCAUCACA | 29989 | ACAAAGUUCUGUGA | 32031 | Neuron |
| 148b-3p | GAACUUUGU | | UGCACUGA | | |
| hsa-miR- | AAGUUCUGUUAU | 29990 | GCCUGAGUGUAUAA | 32032 | Neuron |
| 148b-5p | ACACUCAGGO | | CAGAACUU | | |
| hsa-miR- | CUGGCCCUCUCUG | 29991 | ACGGAAGGGCAGAG | 32033 | Neuron, blood |
| 328 | CCCUUCCGU | | AGGGCCAG | | |
| hsa-miR- | CUAGACUGAAGC | 29992 | CCUCAAGGAGCUUC | 32034 | Neuron, fetal liver |
| 151a-3p | UCCUUGAGG | | AGUCUAG | | |
| hsa-miR- | UCGAGGAGCUCAC | 29993 | ACUAGACUGUGAGC | 32035 | Neuron, fetal liver |
| 15la-5p | AGUCUAGU | | UCCUCGA | | |
| hsa-miR- | CACAUUACACGGU | 29994 | AGAGGUCGACCGUG | 32036 | Neurons |
| 323a-3p | CGACCUCU | | UAAUGUG | | |
| hsa-miR- | AGGUGGUCCGUG | 29995 | GCGAACGCGCCACG | 32037 | Neurons |
| 323a-5p | GCGCGUUCGC | | GACCACCU | | |
| hsa-miR- | CGCAUCCCCUAGG | 29996 | ACACCAAUGCCCUA | 32038 | Neurons |
| 324-5p | GCAUUGGUGU | | GGGGAUGCG | | |
| hsa-miR- | CCUCUGGGCCCUU | 29997 | CUGGAGGAAGGGCC | 32039 | Neurons |
| 326 | CCUCCAG | | CAGAGG | | |
| hsa-miR- | CCUAGUAGGUGU | 29998 | ACACUUACUGGACA | 32040 | Neurons, placenta |
| 325 | CCAGUAAGUGU | | CCUACUAGG | | |
| hsa-miR- | ACGGUGCUGGAU | 29999 | AAAGGCCACAUCCA | 32041 | Oligodendrocytes |
| 1250 | GUGGCCUUU | | GCACCGU | | |
| hsa-miR- | UCAGCACCAGGAU | 30000 | CUCCAACAAUAUCC | 32042 | Oligodendrocytes |
| 3065-3p | AUUGUUGGAG | | UGGUGCUGA | | |
| hsa-miR- | UCAACAAAAUCAC | 30001 | UCCAGCAUCAGUGA | 32043 | Oligodendrocytes |
| 3065-5p | UGAUGCUGGA | | UUUUGUUGA | | |
| hsa-miR- | AACAAUAUCCUG | 30002 | CACUCAGCACCAGG | 32044 | Oligodendrocytes |
| 338-5p | GUGCUGAGUG | | AUAUUGUU | | |
| hsa-miR- | GGCAGGUUCUCAC | 30003 | CCUAGAGAGGGUGA | 32045 | Oligodendrocytes |
| 657 | CCUCUCUAGG | | GAACCUGCC | | |
| hsa-miR- | GACACGGGCGACA | 30004 | GGGCCGCAGCUGUC | 32046 | Oocyte |
| 602 | GCUGCGGCCC | | GCCCGUGUC | | |
| hsa-miR- | AUGUAUGUGUGC | 30005 | CAUGCACAUGCACA | 32047 | Oocyte and prostate |
| 297 | AUGUGCAUG | | CAUACAU | | |
| hsa-miR- | UAUACAAGGGCA | 30006 | AGAGAGAGUCUGCC | 32048 | Osteoblast |
| 300 | GACUCUCUCU | | CUUGUAUA | | |
| hsa-miR- | GGCGGCGGCGGA | 30007 | CCCCCGCCUCCGCC | 32049 | Osteoblast |
| 3960 | GGCGGGGG | | GCCGCC | | |
| hsa-miR- | GCAGGUGCUCACU | 30008 | AGGAGGACAAGUGA | 32050 | Osteoblast |
| 764 | UGUCCUCCU | | GCACCUGC | | |
| hsa-miR- | GGGGCCUGGCGG | 30009 | CCGCCCACCGCCAG | 32051 | Osteoblasts |
| 2861 | UGGGCGG | | GCCCC | | |
| hsa-miR- | GCCCAAAGGUGA | 30010 | CCCAAAAAAUUCAC | 32052 | Osteoblasts, heart |
| 186-3p | AUUUUUUGGG | | CUUUGGGC | | |
| hsa-miR- | CAAAGAAUUCUCC | 30011 | AGCCCAAAAGGAGA | 32053 | Osteoblasts, heart |
| 186-5p | UUUUGGGCU | | AUUCUUUG | | |
| hsa-miR- | CUGCAAUGUAAG | 30012 | GUAAGAAGUGCUUA | 32054 | Osteogenic cells |
| 106a-3p | CACUUCUUAC | | CAUUGCAG | | |
| hsa-miR- | AAAAGUGCUUAC | 30013 | CUACCUGCACUGUA | 32055 | Osteogenic cells |
| 106a-5p | AGUGCAGGUAG | | AGCACUUUU | | |
| hsa-miR- | ACUGUAGUAUGG | 30014 | CUGGAAGUGCCCAU | 32056 | Osteogenic cells |
| 20b-3p | GCACUUCCAG | | ACUACAGU | | |
| hsa-miR- | CAAAGUGCUCAU | 30015 | CUACCUGCACUAUG | 32057 | Osteogenic cells |
| 20b-5p | AGUGCAGGUAG | | AGCACUUUG | | |
| hsa-miR- | GGGGUAUUGUUU | 30016 | CCUGGCAGCGGAAA | 32058 | Ovary |
| 503-3p | CCGCUGCCAGG | | CAAUACCCC | | |
| hsa-miR- | UAGCAGCGGGAA | 30017 | CUGCAGAACUGUUC | 32059 | Ovary |
| 503-5p | CAGUUCUGCAG | | CCGCUGCUA | | |
| hsa-miR- | CGAGCCUCAAGCA | 30018 | AAGUCCCUUGCUUG | 32060 | Ovary, female |
| 2114-3p | AGGGACUU | | AGGCUCG | | reproductive tract |
| hsa-miR- | UAGUCCCUUCCUU | 30019 | GACCGCUUCAAGGA | 32061 | Ovary, female |
| 2114-5p | GAAGCGGUC | | AGGGACUA | | reproductive tract |
| hsa-miR- | ACUGGACUUGGA | 30020 | CCUUCUGACUCCAA | 32062 | Ovary, lipid |
| 378a-3p | GUCAGAAGG | | GUCCAGU | | metabolism |
| hsa-miR- | CUCCUGACUCCAG | 30021 | ACACAGGACCUGGA | 32063 | Ovary, |
| 378a-5p | GUCCUGUGU | | GUCAGGAG | | placenta/trophoblast |
| | | | | | lipid metabolism |
| hsa-miR- | UUUGUUCGUUCG | 30022 | UCACGCGAGCCGAA | 32064 | Pancreas (islet) |
| 375 | GCUCGCGUGA | | CGAACAAA | | |
| hsa-miR- | ACGGAUGUUUGA | 30023 | UAGCACAUGCUCAA | 32065 | Pancreatic cells |
| 105-3p | GCAUGUGCUA | | ACAUCCGU | | |
| hsa-miR- | UCAAAUGCUCAG | 30024 | ACCACAGGAGUCUG | 32066 | Pancreatic cells |
| 105-5p | ACUCCUGUGGU | | AGCAUUUGA | | |
| hsa-miR- | CGGCAACAAGAA | 30025 | CUCAGGCAGUUUCU | 32067 | Pancreatic cells, |
| 196a-3p | ACUGCCUGAG | | UGUUGCCG | | endometrial tissues, |
| | | | | | mesenchymal stem |
| | | | | | cells |
| hsa-miR- | UAGGUAGUUUCA | 30026 | CCCAACAACAUGAA | 32068 | Pancreatic cells, |
| 196a-5p | UGUUGUUGGG | | ACUACCUA | | endometrial tissues, |
| | | | | | mesenchymal stem |
| | | | | | cells |
| hsa-miR- | CAAUGUUUCCACA | 30027 | GUGAUGCACUGUGG | 32069 | Pancreatic islet, |
| 33a-3p | GUGCAUCAC | | AAACAUUG | | lipid metabolism |
| hsa-miR- | GUGCAUUGUAGU | 30028 | UGCAAUGCAACUAC | 32070 | Pancreatic islet, |
| 33a-5p | UGCAUUGCA | | AAUGCAC | | lipid metabolism |
| hsa-miR- | AUCCCACCUCUGC | 30029 | UGGUGGCAGAGGUG | 32071 | Periodontal tissue |
| 1260a | CACCA | | GGAU | | |
| hsa-miR- | AUCCCACCACUGC | 30030 | AUGGUGGCAGUGGU | 32072 | Periodontal tissue |
| 1260b | CACCAU | | GGGAU | | |
| hsa-miR- | ACCUUCCUCUCCA | 30031 | AAAGACCCAUGGAG | 32073 | Peripheral blood |
| 3667-3p | UGGGUCUUU | | AGGAAGGU | | |
| hsa-miR- | AAAGACCCAUUG | 30032 | ACCUUCUCCUCAAU | 32074 | Peripheral blood |
| 3667-5p | AGGAGAAGGU | | GGGUCUUU | | |
| hsa-miR- | AAUGUAGAGAUU | 30033 | AUUUUGAUCAAUCU | 32075 | Peripheral blood |
| 3668 | GAUCAAAAU | | CUACAUU | | |
| hsa-miR- | ACGGAAUAUGUA | 30034 | UAUAUUCCGUAUAC | 32076 | Peripheral blood |
| 3669 | UACGGAAUAUA | | AUAUUCCGU | | |
| hsa-miR- | AGAGCUCACAGCU | 30035 | UAGAGAAGGACAGC | 32077 | Peripheral blood |
| 3670 | GUCCUUCUCUA | | UGUGAGCUCU | | |
| hsa-miR- | AUCAAAUAAGGA | 30036 | UGCAGACUAGUCCU | 32078 | Peripheral blood |
| 3671 | CUAGUCUGCA | | UAUUUGAU | | |
| hsa-miR- | AUGAGACUCAUG | 30037 | AAGAUGUUUUACAU | 32079 | Peripheral blood |
| 3672 | UAAAACAUCUU | | GAGUCUCAU | | |
| hsa-miR- | AUGGAAUGUAUA | 30038 | UAUUCCGUAUAUAC | 32080 | Peripheral blood |
| 3673 | UACGGAAUA | | AUUCCAU | | |
| hsa-miR- | AUUGUAGAACCU | 30039 | GGCCAAUCUUAGGU | 32081 | Peripheral blood |
| 3674 | AAGAUUGGCC | | UCUACAAU | | |
| hsa-miR- | CAUCUCUAAGGA | 30040 | UUGGGGGAGUUCCU | 32082 | Peripheral blood |
| 3675-3p | ACUCCCCCAA | | UAGAGAUG | | |
| hsa-miR- | UAUGGGGCUUCU | 30041 | GAAAUCUCUACAGA | 32083 | Peripheral blood |
| 3675-5p | GUAGAGAUUUC | | AGCCCCAUA | | |
| hsa-miR- | CCGUGUUUCCCCC | 30042 | AAAGCGUGGGGGAA | 32084 | Peripheral blood |
| 3676-3p | ACGCUUU | | ACACGG | | |
| hsa-miR- | AGGAGAUCCUGG | 30043 | AACCCAGGAUCUCC | 32085 | Peripheral blood |
| 3676-5p | GUU | | U | | |
| hsa-miR- | CUCGUGGGCUCUG | 30044 | GGCCGUGGCCAGAG | 32086 | Peripheral blood |
| 3677-3p | GCCACGGCC | | CCCACGAG | | |
| hsa-miR- | CAGUGGCCAGAGC | 30045 | CACUGCAGGGCUCU | 32087 | Peripheral blood |
| 3677-5p | CCUGCAGUG | | GGCCACUG | | |
| hsa-miR- | CUGCAGAGUUUG | 30046 | CCGGUCCGUACAAA | 32088 | Peripheral blood |
| 3678-3p | UACGGACCGG | | CUCUGCAG | | |
| hsa-miR- | UCCGUACAAACUC | 30047 | CACAGCAGAGUUUG | 32089 | Peripheral blood |
| 3678-5p | UGCUGUG | | UACGGA | | |
| hsa-miR- | CUUCCCCCCAGUA | 30048 | GAUGAAGAUUACUG | 32090 | Peripheral blood |
| 3679-3p | AUCUUCAUC | | GGGGGAAG | | |
| hsa-miR- | UGAGGAUAUGGC | 30049 | UCCCCUUCCCUGCC | 32091 | Peripheral blood |
| 3679-5p | AGGGAAGGGGA | | AUAUCCUCA | | |
| hsa-miR- | UUUUGCAUGACCC | 30050 | CCUACUCCCAGGGU | 32092 | Peripheral blood |
| 3680-3p | UGGGAGUAGG | | CAUGCAAAA | | |
| hsa-miR- | GACUCACUCACAG | 30051 | UGCACAAUCCUGUG | 32093 | Peripheral blood |
| 3680-5p | GAUUGUGCA | | AGUGAGUC | | |
| hsa-miR- | ACACAGUGCUUCA | 30052 | AGUAGUGGAUGAAG | 32094 | Peripheral blood |
| 3681-3p | UCCACUACU | | CACUGUGU | | |
| hsa-miR- | UAGUGGAUGAUG | 30053 | GCACAGAGUGCAUC | 32095 | Peripheral blood |
| 3681-5p | CACUCUGUGC | | AUCCACUA | | |
| hsa-miR- | UGAUGAUACAGG | 30054 | CUACCUCCACCUGU | 32096 | Peripheral blood |
| 3682-3p | UGGAGGUAG | | AUCAUCA | | |
| hsa-miR- | CUACUUCUACCUG | 30055 | AUGAUAACACAGGU | 32097 | Peripheral blood |
| 3682-5p | UGUUAUCAU | | AGAAGUAG | | |
| hsa-miR- | UGCGACAUUGGA | 30056 | UGAUACUACUUCCA | 32098 | Peripheral blood |
| 3683 | AGUAGUAUCA | | AUGUCGCA | | |
| hsa-miR- | UUAGACCUAGUA | 30057 | AAGGACGUGUACUA | 32099 | Peripheral blood |
| 3684 | CACGUCCUU | | GGUCUAA | | |
| hsa-miR- | UUUCCUACCCUAC | 30058 | AGUCUUCAGGUAGG | 32100 | Peripheral blood |
| 3685 | CUGAAGACU | | GUAGGAAA | | |
| hsa-miR- | AUCUGUAAGAGA | 30059 | UCAUUUACUUUCUC | 32101 | Peripheral blood |
| 3686 | AAGUAAAUGA | | UUACAGAU | | |
| hsa-miR- | CCCGGACAGGCGU | 30060 | ACGUCGCACGAACG | 32102 | Peripheral blood |
| 3687 | UCGUGCGACGU | | CCUGUCCGGG | | |
| hsa-miR- | ACCUGGACCCAGC | 30061 | CUUUGUCUACGCUG | 32103 | Peripheral blood |
| 3690 | GUAGACAAAG | | GGUCCAGGU | | |
| hsa-miR- | ACCAAGUCUGCGU | 30062 | GAGAGGAUGACGCA | 32104 | Peripheral blood |
| 3691-3p | CAUCCUCUC | | GACUUGGU | | |
| hsa-miR- | AGUGGAUGAUGG | 30063 | GUACCGAGUCUCCA | 32105 | Peripheral blood |
| 3691-5p | AGACUCGGUAC | | UCAUCCACU | | |
| hsa-miR- | GUUCCACACUGAC | 30064 | ACUUCUGCAGUGUC | 32106 | Peripheral blood |
| 3692-3p | ACUGCAGAAGU | | AGUGUGGAAC | | |
| hsa-miR- | CCUGCUGGUCAGG | 30065 | CAGUAUCCACUCCU | 32107 | Peripheral blood |
| 3692-5p | AGUGGAUACUG | | GACCAGCAGG | | |
| hsa-miR- | GAGGGUCUUGGG | 30066 | GUCACAUCCCUCCC | 32108 | Placenta |
| 1182 | AGGGAUGUGAC | | AAGACCCUC | | |
| hsa-miR- | AUAUACAGGGGG | 30067 | AUAAGAGUCUCCCC | 32109 | Placenta |
| 1185-1-3p | AGACUCUUAU | | CUGUAUAU | | |
| hsa-miR- | AUAUACAGGGGG | 30068 | AUGAGAGUCUCCCC | 32110 | Placenta |
| 1185-2-3p | AGACUCUCAU | | CUGUAUAU | | |
| hsa-miR- | AGAGGAUACCCU | 30069 | AACAUACAAAGGGU | 32111 | Placenta |
| 1185-5p | UUGUAUGUU | | AUCCUCU | | |
| hsa-miR- | UCUACAAAGGAA | 30070 | AGAAAGCGCUUUCC | 32112 | Placenta |
| 1283 | AGCGCUUUCU | | UUUGUAGA | | |
| hsa-miR- | UCAAAACUGAGG | 30071 | AGAAAAUGCCCCUC | 32113 | Placenta |
| 1323 | GGCAUUUUCU | | AGUUUUGA | | |
| hsa-miR- | UUCUCCAAAAGA | 30072 | CAGAAAGUGCUUUC | 32114 | Placenta |
| 515-5p | AAGCACUUUCUG | | UUUUGGAGAA | | |
| hsa-miR- | UUCUCGAGGAAA | 30073 | GAAAGUGCUUCUUU | 32115 | Placenta |
| 516a-5p | GAAGCACUUUC | | CCUCGAGAA | | |
| hsa-miR- | CCUCUAGAUGGA | 30074 | AGACAGUGCUUCCA | 32116 | Placenta |
| 517-5p | AGCACUGUCU | | UCUAGAGG | | |
| hsa-miR- | AUCGUGCAUCCCU | 30075 | ACACUCUAAAGGGA | 32117 | Placenta |
| 517a-3p | UUAGAGUGU | | UGCACGAU | | |
| hsa-miR- | AUCGUGCAUCCCU | 30076 | ACACUCUAAAGGGA | 32118 | Placenta |
| 517b-3p | UUAGAGUGU | | UGCACGAU | | |
| hsa-miR- | AUCGUGCAUCCUU | 30077 | ACACUCUAAAAGGA | 32119 | Placenta |
| 517c-3p | UUAGAGUGU | | UGCACGAU | | |
| hsa-miR- | CAAAGCGCUCCCC | 30078 | ACCUCUAAAGGGGA | 32120 | Placenta |
| 518b | UUUAGAGGU | | GCGCUUUG | | |
| hsa-miR- | CAAAGCGCUUCUC | 30079 | ACACUCUAAAGAGA | 32121 | Placenta |
| 518c-3p | UUUAGAGUGU | | AGCGCUUUG | | |
| hsa-miR- | UCUCUGGAGGGA | 30080 | CAGAAAGUGCUUCC | 32122 | Placenta |
| 518c-5p | AGCACUUUCUG | | CUCCAGAGA | | |
| hsa-miR- | GAAAGCGCUUCUC | 30081 | CCUCUAAAGAGAAG | 32123 | Placenta |
| 518f-3p | UUUAGAGG | | CGCUUUC | | |
| hsa-miR- | CUCUAGAGGGAA | 30082 | GAGAAAGUGCUUCC | 32124 | Placenta |
| 518f-5p | GCACUUUCUC | | CUCUAGAG | | |
| hsa-miR- | AAAGUGCAUCCU | 30083 | ACACUCUAAAAGGA | 32125 | Placenta |
| 519a-3p | UUUAGAGUGU | | UGCACUUU | | |
| hsa-miR- | CUCUAGAGGGAA | 30084 | CAGAAAGCGCUUCC | 32126 | Placenta |
| 519a-5p | GCGCUUUCUG | | CUCUAGAG | | |
| hsa-miR- | CAAAGUGCCUCCC | 30085 | CACUCUAAAGGGAG | 32127 | Placenta |
| 519d | UUUAGAGUG | | GCACUUUG | | |
| hsa-miR- | AAGUGCCUCCUUU | 30086 | AACACUCUAAAAGG | 32128 | Placenta |
| 519e-3p | UAGAGUGUU | | AGGCACUU | | |
| hsa-miR- | UUCUCCAAAAGG | 30087 | GAAAGUGCUCCCUU | 32129 | Placenta |
| 519e-5p | GAGCACUUUC | | UUGGAGAA | | |
| hsa-miR- | AAAGUGCUUCCCU | 30088 | ACAGUCCAAAGGGA | 32130 | Placenta |
| 520a-3p | UUGGACUGU | | AGCACUUU | | |
| hsa-miR- | CUCCAGAGGGAA | 30089 | AGAAAGUACUUCCC | 32131 | Placenta |
| 520a-5p | GUACUUUCU | | UCUGGAG | | |
| hsa-miR- | ACAAAGUGCUUCC | 30090 | ACUCUAAAGGGAAG | 32132 | Placental specific |
| 520h | CUUUAGAGU | | CACUUUGU | | |
| hsa-miR- | CUACAAAGGGAA | 30091 | GAGAAAGUGCUUCC | 32133 | Placental specific |
| 524-5p | GCACUUUCUC | | CUUUGUAG | | |
| hsa-miR- | GAAGGOGCUUCCC | 30092 | CGCUCUAAAGGGAA | 32134 | Placental specific |
| 525-3p | UUUAGAGCG | | GCGCCUUC | | |
| hsa-miR- | CUCCAGAGGGAU | 30093 | AGAAAGUGCAUCCC | 32135 | Placental specific |
| 525-5p | GCACUUUCU | | UCUGGAG | | |
| hsa-miR- | CUCUAGAGGGAA | 30094 | CAGAAAGUGCUUCC | 32136 | Placental specific |
| 526a | GCACUUUCUG | | CUCUAGAG | | |
| hsa-miR- | GAAAGUGCUUCC | 30095 | GCCUCUAAAAGGAA | 32137 | Placental specific |
| 526b-3p | UUUUAGAGGC | | GCACUUUC | | |
| hsa-miR- | CUCUUGAGGGAA | 30096 | ACAGAAAGUGCUUC | 32138 | Placental specific |
| 526b-5p | GCACUUUCUGU | | CCUCAAGAG | | |
| hsa-miR- | ACUGGACUUAGG | 30097 | GCCUUCUGACCCUA | 32139 | Plasma |
| 422a | GUCAGAAGGC | | AGUCCAGU | | |
| hsa-miR- | AAACAAACAUGG | 30098 | AAGAAGUGCACCAU | 32140 | Platelet |
| 495-3p | UGCACUUCUU | | GUUUGUUU | | |
| hsa-miR- | GAAGUUGCCCAU | 30099 | CGAAAAUAACAUGG | 32141 | Platelet |
| 495-5p | GUUAUUUUCG | | GCAACUUC | | |
| hsa-miR- | AAUCAUUCACGG | 30100 | AAGUGUUGUCCGUG | 32142 | Renal epithelial |
| 382-3p | ACAACACUU | | AAUGAUU | | cells |
| hsa-miR- | GAAGUUGUUCGU | 30101 | CGAAUCCACCACGA | 32143 | Renal epithelial |
| 382-5p | GGUGGAUUCG | | ACAACUUC | | cells |
| hsa-miR- | CCUCCGUGUUACC | 30102 | CUAGAGGACAGGUA | 32144 | Reproductive tracts |
| 3605-3p | UGUCCUCUAG | | ACACGGAGG | | |
| hsa-miR- | UGAGGAUGGAUA | 30103 | GGCUUCCUUGCUAU | 32145 | Reproductive tracts |
| 3605-5p | GCAAGGAAGCC | | CCAUCCUCA | | |
| hsa-miR- | UUCAGAUCCCAGC | 30104 | AGAGGCACCGCUGG | 32146 | Salivary gland |
| 5100 | GGUGCCUCU | | GAUCUGAA | | |
| hsa-miR- | GUCCUAGGAGGC | 30105 | CAGAGGAGCCUCCU | 32147 | Salivary gland |
| 5571-3p | UCCUCUG | | AGGAC | | |
| hsa-miR- | CAAUUCUCAAAG | 30106 | GGGAGGCUCCUUUG | 32148 | Salivary gland |
| 5571-5p | GAGCCUCCC | | AGAAUUG | | |
| hsa-miR- | GUUGGGGUGCAG | 30107 | AGCAGACCCCUGCA | 32149 | Salivary gland |
| 5572 | GGGUCUGCU | | CCCCAAC | | |
| hsa-miR- | UUUCCGGCUCGCG | 30108 | ACACACCCACGCGA | 32150 | Sarcoma |
| 1180 | UGGGUGUGU | | GCCGGAAA | | |
| hsa-miR- | UGAGUGUGUGUG | 30109 | ACACACUCACACAC | 32151 | Semen |
| 574-5p | UGUGAGUGUGU | | ACACACUCA | | |
| hsa-miR- | AGUGGGAGGCCA | 30110 | UGCCGUGCCCUGGC | 32152 | Serum |
| 1233-1-5p | GGGCACGGCA | | CUCCCACU | | |
| hsa-miR- | UGAGCCCUGUCCU | 30111 | CUGCGGGAGGACAG | 32153 | Serum |
| 1233-3p | CCCGCAG | | GGCUCA | | |
| hsa-miR- | AAGUGCCGCCAUC | 30112 | ACACUCAAAAGAUG | 32154 | Serum |
| 37la-3p | UUUUGAGUGU | | GCGGCACUU | | |
| hsa-miR- | ACUCAAACUGUG | 30113 | AGUGCCCCCACAGU | 32155 | Serum |
| 371a-5p | GGGGCACU | | UUGAGU | | |
| hsa-miR- | AAGUGCCCCCACA | 30114 | GCACUCAAACUGUG | 32156 | Serum |
| 371b-3p | GUUUGAGUGC | | GGGGCACUU | | |
| hsa-miR- | ACUCAAAAGAUG | 30115 | AAAGUGCCGCCAUC | 32157 | Serum |
| 371b-5p | GCGGCACUUU | | UUUUGAGU | | |
| hsa-miR- | AAACCUGUGUUG | 30116 | GACUCUUGAACAAC | 32158 | Serum |
| 649 | UUCAAGAGUC | | ACAGGUUU | | |
| hsa-miR- | ACAGUAGAGGGA | 30117 | CUGCGAUUCCUCCO | 32159 | Skin |
| 936 | GGAAUCGCAG | | UCUACUGU | | |
| hsa-miR- | GUGAAAUGUUUA | 30118 | CUAGUGGUCCUAAA | 32160 | Skin (epithelium) |
| 203a | GGACCACUAG | | CAUUUCAC | | |
| hsa-miR- | UUGAACUGUUAA | 30119 | UCCAGUGGUUCUUA | 32161 | Skin specific |
| 203b-3p | GAACCACUGGA | | ACAGUUCAA | | (epithelium) |
| hsa-miR- | UAGUGGUCCUAA | 30120 | UGUGAAAUGUUUAG | 32162 | Skin specific |
| 203b-5p | ACAUUUCACA | | GACCACUA | | (epithelium) |
| hsa-miR- | UGCAGGACCAAG | 30121 | AGGGCUCAUCUUGG | 32163 | Smooth muscle |
| 1286 | AUGAGCCCU | | UCCUGCA | | |
| hsa-miR- | CUCCCACAUGCAG | 30122 | UGCAAACCCUGCAU | 32164 | Smooth muscle, |
| 188-3p | GGUUUGCA | | GUGGGAG | | central nervous |
| | | | | | system |
| hsa-miR- | CAUCCCUUGCAUG | 30123 | CCCUCCACCAUGCA | 32165 | Smooth muscle, |
| 188-5p | GUGGAGGG | | AGGGAUG | | central nervous |
| | | | | | system |
| hsa-miR- | AAAAUGAAAUGA | 30124 | UGGGCUGGGCUCAU | 32166 | Solid tumor |
| 3646 | GCCCAGCCCA | | UUCAUUUU | | |
| hsa-miR- | AGCCGCGGGGAUC | 30125 | CCCUCGGCGAUCCC | 32167 | Solid tumor |
| 3648 | GCCGAGGG | | CGCGGCU | | |
| hsa-miR- | AGGGACCUGAGU | 30126 | CUUAGACACUCAGG | 32168 | Solid tumor |
| 3649 | GUCUAAG | | UCCCU | | |
| hsa-miR- | AGGUGUGUCUGU | 30127 | GGACUCUACAGACA | 32169 | Solid tumor |
| 3650 | AGAGUCC | | CACCU | | |
| hsa-miR- | CAUAGCCCGGUCG | 30128 | UCAUGUACCAGCGA | 32170 | Solid tumor |
| 3651 | CUGGUACAUGA | | CCGGGCUAUG | | |
| hsa-miR- | CGGCUGGAGGUG | 30129 | UCCUCACACCUCCA | 32171 | Solid tumor |
| 3652 | UGAGGA | | GCCG | | |
| hsa-miR- | CUAAGAAGUUGA | 30130 | CUUCAGUCAACUUC | 32172 | Solid tumor |
| 3653 | CUGAAG | | UUAG | | |
| hsa-miR- | GACUGGACAAGC | 30131 | UUCCUCAGCUUGUC | 32173 | Solid tumor |
| 3654 | UGAGGAA | | CAGUC | | |
| hsa-miR- | GCUUGUCGCUGCG | 30132 | AGCAACACCGCAGC | 32174 | Solid tumor |
| 3655 | GUGUUGCU | | GACAAGC | | |
| hsa-miR- | GGCGGGUGCGGG | 30133 | CCACCCCCGCACCC | 32175 | Solid tumor |
| 3656 | GGUGG | | GCC | | |
| hsa-miR- | UGUGUCCCAUUA | 30134 | AAUCACCAAUAAUG | 32176 | Solid tumor |
| 3657 | UUGGUGAUU | | GGACACA | | |
| hsa-miR- | UUUAAGAAAACA | 30135 | AUCUCCAUGGUGUU | 32177 | Solid tumor |
| 3658 | CCAUGGAGAU | | UUCUUAAA | | |
| hsa-miR- | GCUAUUUCACGAC | 30136 | AACCCUGGUGUCGU | 32178 | Stem cells |
| 138-2-3p | ACCAGGGUU | | GAAAUAGC | | |
| hsa-miR- | AGCUGGUGUUGU | 30137 | CGGCCUGAUUCACA | 32179 | Stem cells |
| 138-5p | GAAUCAGGCCG | | ACACCAGCU | | |
| hsa-miR- | AAUAAUACAUGG | 30138 | AAAGAUCAACCAUG | 32180 | Stem cells |
| 369-3p | UUGAUCUUU | | UAUUAUU | | |
| hsa-miR- | AGAUCGACCGUG | 30139 | GCGAAUAUAACACG | 32181 | Stem cells |
| 369-5p | UUAUAUUCGC | | GUCGAUCU | | |
| hsa-miR- | AAUCAUGUGCAG | 30140 | CAUAUUGGCACUGC | 32182 | Stem cells |
| 96-3p | UGCCAAUAUG | | ACAUGAUU | | |
| hsa-miR- | UUUGGCACUAGC | 30141 | AGCAAAAAUGUGCU | 32183 | Stem cells |
| 96-5p | ACAUUUUUGCU | | AGUGCCAAA | | |
| hsa-miR- | UCCUGUACUGAGC | 30142 | CUCGGGGCAGCUCA | 32184 | Stem cells (adipose) |
| 486-5p | UGCCCCGAG | | GUACAGGA | | |
| hsa-miR- | GCUACUUCACAAC | 30143 | GGCCCUGGUGUUGU | 32185 | Stem cells, |
| 138-1-3p | ACCAGGGCC | | GAAGUAGC | | epidermal cells |
| | | | | | (keratinocytes) |
| hsa-miR- | CAUCAUCGUCUCA | 30144 | AGACUCAUUUGAGA | 32186 | Stem cells, placenta |
| 136-3p | AAUGAGUCU | | CGAUGAUG | | |
| hsa-miR- | ACUCCAUUUGUU | 30145 | UCCAUCAUCAAAAC | 32187 | Stem cells, placenta |
| 136-5p | UUGAUGAUGGA | | AAAUGGAGU | | |
| hsa-miR- | CUCCUACAUAUUA | 30146 | UGUUAAUGCUAAUA | 32188 | T/B cells, |
| 155-3p | GCAUUAACA | | UGUAGGAG | | monocytes, breast |
| hsa-miR- | UUAAUGCUAAUC | 30147 | ACCCCUAUCACGAU | 32189 | T/B cells, |
| 155-5p | GUGAUAGGGGU | | UAGCAUUAA | | monocytes, breast |
| hsa-miR- | AGAUCAGAAGGU | 30148 | AGCCACAAUCACCU | 32190 | Testes, brain |
| 383 | GAUUGUGGCU | | UCUGAUCU | | (medulla) |
| hsa-miR- | UACUGCAGACGU | 30149 | CAUGAUUGCCACGU | 32191 | Testis |
| 509-3-5p | GGCAAUCAUG | | CUGCAGUA | | |
| hsa-miR- | GUUAGGGCCAAC | 30150 | CCAAGAGAUGUUGG | 32192 | T-Lymphocytes |
| 2909 | AUCUCUUGG | | CCCUAAC | | |
| hsa-miR- | AACAUAGAGGAA | 30151 | ACGUGGAAUUUCCU | 32193 | Trophoblast |
| 376c-3p | AUUCCACGU | | CUAUGUU | | |
| hsa-miR- | GGUGGAUAUUCC | 30152 | AACAUAGAAGGAAU | 32194 | Trophoblast |
| 376c-5p | UUCUAUGUU | | AUCCACC | | |
| hsa-miR- | UCAUAGOCCUGUA | 30153 | AGCAGCAUUGUACA | 32195 | Variety of cells and |
| 103b | CAAUGCUGCU | | GGGCUAUGA | | tissues |
| hsa-miR- | UAACACUGUCUG | 30154 | CCAUCUUUACCAGA | 32196 | Variety of cells and |
| 141-3p | GUAAAGAUGG | | CAGUGUUA | | tissues |
| hsa-miR- | CAUCUUCCAGUAC | 30155 | UCCAACACUGUACU | 32197 | Variety of cells and |
| 141-5p | AGUGUUGGA | | GGAAGAUG | | tissues |
| hsa-miR- | AACUGGCCUACAA | 30156 | ACUGGGACUUUGUA | 32198 | Variety of cells and |
| 193a-3p | AGUCCCAGU | | GGCCAGUU | | tissues |
| hsa-miR- | UGGGUCUUUGCG | 30157 | UCAUCUCGCCCGCA | 32199 | Variety of cells and |
| 193a-5p | GGCGAGAUGA | | AAGACCCA | | tissues |
| hsa-miR- | AUGACCUAUGAA | 30158 | GUCUGUCAAUUCAU | 32200 | Variety of cells and |
| 215 | UUGACAGAC | | AGGUCAU | | tissues |
| hsa-miR- | AAGCUGCCAGUU | 30159 | ACAGUUCUUCAACU | 32201 | Variety of cells and |
| 22-3p | GAAGAACUGU | | GGCAGCUU | | tissues |
| hsa-miR- | AGUUCUUCAGUG | 30160 | UAAAGCUUGCCACU | 32202 | Variety of cells and |
| 22-5p | GCAAGCUUUA | | GAAGAACU | | tissues |
| hsa-miR- | ACAGAUUCGAUU | 30161 | AUUCCCCUAGAAUC | 32203 | Variety of tissues |
| 10b-3p | CUAGGGGAAU | | GAAUCUGU | | and cells |
| hsa-miR- | UACCCUGUAGAAC | 30162 | CACAAAUUCGGUUC | 32204 | Variety of tissues |
| 10b-5p | CGAAUUUGUG | | UACAGGGUA | | and cells |
| hsa-miR- | UAGCAGCACGUA | 30163 | CGCCAAUAUUUACG | 32205 | Variety of tissues, |
| 16-5p | AAUAUUGGCG | | UGCUGCUA | | blood |
| hsa-miR- | UGAGAUGAAGCA | 30164 | GAGCUACAGUGCUU | 32206 | Vascular smooth |
| 143-3p | CUGUAGCUC | | CAUCUCA | | muscle |
| hsa-miR- | GGUGCAGUGCUG | 30165 | ACCAGAGAUGCAGC | 32207 | Vascular smooth |
| 143-5p | CAUCUCUGGU | | ACUGCACC | | muscle, T-cells |
| hsa-miR- | UUGCUCACUGUUC | 30166 | CUAGGGAAGAACAG | 32208 | |
| 1178-3p | UUCCCUAG | | UGAGCAA | | |
| hsa-miR- | CAGGGUCAGCUG | 30167 | CAUGCUCAGCUGAC | 32209 | |
| 1178-5p | AGCAUG | | CCUG | | |
| hsa-miR- | AAGCAUUCUUUC | 30168 | CCAACCAAUGAAAG | 32210 | |
| 1179 | AUUGGUUGG | | AAUGCUU | | |
| hsa-miR- | CCGUCGCCGCCAC | 30169 | CGGCUCGGGUGGCG | 32211 | |
| 1181 | CCGAGCCG | | GCGACGG | | |
| hsa-miR- | CACUGUAGGUGA | 30170 | UGCCCACUCUCACC | 32212 | |
| 1183 | UGGUGAGAGUGG | | AUCACCUACAGUG | | |
| | GCA | | | | |
| hsa-miR- | GGGAUGGUAGAC | 30171 | GCACGUCACCGGUC | 32213 | |
| 1193 | CGGUGACGUGC | | UACCAUCCC | | |
| hsa-miR- | UAGGACACAUGG | 30172 | AGAAGUAGACCAUG | 32214 | |
| 1197 | UCUACUUCU | | UGUCCUA | | |
| hsa-miR- | CUCCUGAGCCAUU | 30173 | GAGGCUCAGAAUGG | 32215 | |
| 1200 | CUGAGCCUC | | CUCAGGAG | | |
| hsa-miR- | GUGCCAGCUGCAG | 30174 | CUCCCCCACUGCAG | 32216 | |
| 1202 | UGGGGGAG | | CUGGCAC | | |
| hsa-miR- | CCCGGAGCCAGGA | 30175 | GAGCUGCAUCCUGG | 32217 | |
| 1203 | UGCAGCUC | | CUCCGGG | | |
| hsa-miR- | UCGUGGCCUGGUC | 30176 | AUAAUGGAGACCAG | 32218 | |
| 1204 | UCCAUUAU | | GCCACGA | | |
| hsa-miR- | UCUGCAGGGUUU | 30177 | CUCAAAGCAAACCC | 32219 | |
| 1205 | GCUUUGAG | | UGCAGA | | |
| hsa-miR- | UGUUCAUGUAGA | 30178 | GCUUAAACAUCUAC | 32220 | |
| 1206 | UGUUUAAGC | | AUGAACA | | |
| hsa-miR- | UCAGCUGGCCCUC | 30179 | GAAAUGAGGGCCAG | 32221 | |
| 1207-3p | AUUUC | | CUGA | | |
| hsa-miR- | UGGCAGGGAGGC | 30180 | CCCCUCCCAGCCUC | 32222 | |
| 1207-5p | UGGGAGGGG | | CCUGCCA | | |
| hsa-miR- | UCACUGUUCAGAC | 30181 | UCCGCCUGUCUGAA | 32223 | |
| 1208 | AGGCGGA | | CAGUGA | | |
| hsa-miR- | CCCCACCUCCUCU | 30182 | CUGAGGAGAGAGGA | 32224 | |
| 1224-3p | CUCCUCAG | | GGUGGGG | | |
| hsa-miR- | GUGAGGACUCGG | 30183 | CCACCUCCCGAGUC | 32225 | |
| 1224-5p | GAGGUGG | | CUCAC | | |
| hsa-miR- | UGAGCCCCUGUGC | 30184 | CUGGGGGCGGCACA | 32226 | |
| 1225-3p | CGCCCCCAG | | GGGGCUCA | | |
| hsa-miR- | GUGGGUACGGCCC | 30185 | CCCCCCACUGGGCC | 32227 | |
| 1225-5p | AGUGGGGGG | | GUACCCAC | | |
| hsa-miR- | UCACCAGCCCUGU | 30186 | CUAGGGAACACAGG | 32228 | |
| 1226-3p | GUUCCCUAG | | GCUGGUGA | | |
| hsa-miR- | GUGAGGGCAUGC | 30187 | CCCCAUCCAGGCCU | 32229 | |
| 1226-5p | AGGCCUGGAUGG | | GCAUGCCCUCAC | | |
| | GG | | | | |
| hsa-miR- | CUCUCACCACUGC | 30188 | CUGUGGGAGGGCAG | 32230 | |
| 1229-3p | CCUCCCACAG | | UGGUGAGAG | | |
| hsa-miR- | GUGGGUAGGGUU | 30189 | CGCUCUCCCCCAAA | 32231 | |
| 1229-5p | UGGGGGAGAGCG | | CCCUACCCAC | | |
| hsa-miR- | GUGUCUGGGCGG | 30190 | GCAGCUGUCCGCCC | 32232 | |
| 1231 | ACAGCUGC | | AGACAC | | |
| hsa-miR- | CUUCCUCGUCUGU | 30191 | GGGGCAGACAGACG | 32233 | |
| 1238-3p | CUGCCCC | | AGGAAG | | |
| hsa-miR- | GUGAGUGGGAGC | 30192 | CACACACUGGGGCU | 32234 | |
| 1238-5p | CCCAGUGUGUG | | CCCACUCAC | | |
| hsa-miR- | ACCUUCUUGUAU | 30193 | UUUAGCACAGUGCU | 32235 | |
| 1248 | AAGCACUGUGCU | | UAUACAAGAAGGU | | |
| | AAA | | | | |
| hsa-miR- | GGCGACAAAACG | 30194 | GACAGGGUCUCGUU | 32236 | |
| 1273c | AGACCCUGUC | | UUGUCGCC | | |
| hsa-miR- | UUGCUUGAACCCA | 30195 | UCCACUUCCUGGGU | 32237 | |
| 1273e | GGAAGUGGA | | UCAAGCAA | | |
| hsa-miR- | GGAGAUGGAGGU | 30196 | CACUGCAACCUCCA | 32238 | |
| 1273f | UGCAGUG | | UCUCC | | |
| hsa-miR- | ACCACUGCACUCC | 30197 | CUCAGGCUGGAGUG | 32239 | |
| 1273g-3p | AGCCUGAG | | CAGUGGU | | |
| hsa-miR- | GGUGGUUGAGGC | 30198 | ACUUACUGCAGCCU | 32240 | |
| 1273g-5p | UGCAGUAAGU | | CAACCACC | | |
| hsa-miR- | UCGCCUCCUCCUC | 30199 | GGGAGAGGAGGAGG | 32241 | |
| 1281 | UCCC | | CGA | | |
| hsa-miR- | UCUAUACAGACCC | 30200 | GAAAAGCCAGGGUC | 32242 | |
| 1284 | UGGCUUUUC | | UGUAUAGA | | |
| hsa-miR- | UCUGGGCAACAA | 30201 | AGGUCUCACUUUGU | 32243 | |
| 1285-3p | AGUGAGACCU | | UGCCCAGA | | |
| hsa-miR- | GAUCUCACUUUG | 30202 | CCUGGGCAACAAAG | 32244 | |
| 1285-5p | UUGCCCAGG | | UGAGAUC | | |
| hsa-miR- | UCGCGCCCCGGCU | 30203 | GAACGGGAGCCGGG | 32245 | |
| 1292-3p | CCCGUUC | | GCGCGA | | |
| hsa-miR- | UGGGAACGGGUU | 30204 | CAGCGUCUGCCGGA | 32246 | |
| 1292-5p | CCGGCAGACGCUG | | ACCCGUUCCCA | | |
| hsa-miR- | UUAGGCCGCAGA | 30205 | UCACCCAGAUCUGC | 32247 | |
| 1295a | UCUGGGUGA | | GGCCUAA | | |
| hsa-miR- | AAUAGGCCACGG | 30206 | UUGCCCAGAUCCGU | 32248 | |
| 1295b-3p | AUCUGGGCAA | | GGCCUAUU | | |
| hsa-miR- | CACCCAGAUCUGC | 30207 | AUUAGGCCGCAGAU | 32249 | |
| 1295b-5p | GGCCUAAU | | CUGGGUG | | |
| hsa-miR- | UUAGGGCCCUGGC | 30208 | GGAGAUGGAGCCAG | 32250 | |
| 1296 | UCCAUCUCC | | GGCCCUAA | | |
| hsa-miR- | UUCAUUCGGCUG | 30209 | UACAUCUGGACAGC | 32251 | |
| 1298 | UCCAGAUGUA | | CGAAUGAA | | |
| hsa-miR- | UUGCAGCUGCCUG | 30210 | GAAGUCACUCCCAG | 32252 | |
| 1301 | GGAGUGACUUC | | GCAGCUGCAA | | |
| hsa-miR- | UUGGGACAUACU | 30211 | UUUAGCAUAAGUAU | 32253 | |
| 1302 | UAUGCUAAA | | GUCCCAA | | |
| hsa-miR- | UCUCACUGUAGCC | 30212 | GGGGUUCGAGGCUA | 32254 | |
| 1304-3p | UCGAACCCC | | CAGUGAGA | | |
| hsa-miR- | UUUGAGGCUACA | 30213 | CACAUCUCACUGUA | 32255 | |
| 1304-5p | GUGAGAUGUG | | GCCUCAAA | | |
| hsa-miR- | CAGGGAGGUGAA | 30214 | AUCACAUUCACCUC | 32256 | |
| 1321 | UGUGAU | | CCUG | | |
| hsa-miR- | GAUGAUGCUGCU | 30215 | CAGCAUCAGCAGCA | 32257 | |
| 1322 | GAUGCUG | | UCAUC | | |
| hsa-miR- | CCAGACAGAAUUC | 30216 | GAAAGUGCAUAGAA | 32258 | |
| 1324 | UAUGCACUUUC | | UUCUGUCUGG | | |
| hsa-miR- | CUCCUGGGGCCCG | 30217 | GCGAGAGUGCGGGC | 32259 | |
| 1343 | CACUCUCGC | | CCCAGGAG | | |
| hsa-miR- | CUCCGUUUGCCUG | 30218 | CAGCGAAACAGGCA | 32260 | |
| 1468 | UUUCGCUG | | AACGGAG | | |
| hsa-miR- | CUCGGCGCGGGGC | 30219 | GGAGCCCGCGCCCC | 32261 | |
| 1469 | GCGGGCUCC | | GCGCCGAG | | |
| hsa-miR- | GCCCUCCGCCCGU | 30220 | CGGGGUGCACGGGC | 32262 | |
| 1470 | GCACCCCG | | GGAGGGC | | |
| hsa-miR- | GCCCGCGUGUGGA | 30221 | ACACCUGGCUCCAC | 32263 | |
| 1471 | GCCAGGUGU | | ACGCGGGC | | |
| hsa-miR- | AAAACCGUCUAG | 30222 | ACAACUGUAACUAG | 32264 | |
| 1537 | UUACAGUUGU | | ACGGUUUU | | |
| hsa-miR- | UCCAGUGCCCUCC | 30223 | GGAGAGGAGGGCAC | 32265 | |
| 1825 | UCUCC | | UGGA | | |
| hsa-miR- | UGAGGCAGUAGA | 30224 | AUUCAAUCUACUGC | 32266 | |
| 1827 | UUGAAU | | CUCA | | |
| hsa-miR- | AGGGGCUGGCUU | 30225 | GACCAGAGGAAAGC | 32267 | |
| 185-3p | UCCUCUGGUC | | CAGCCCCU | | |
| hsa-miR- | UGGAGAGAAAGG | 30226 | UCAGGAACUGCCUU | 32268 | |
| 185-5p | CAGUUCCUGA | | UCUCUCCA | | |
| hsa-miR- | UCGUGUCUUGUG | 30227 | CCGGCUGCAACACA | 32269 | |
| 187-3p | UUGCAGCCGG | | AGACACGA | | |
| hsa-miR- | GGCUACAACACAG | 30228 | GCCCGGGUCCUGUG | 32270 | |
| 187-5p | GACCCGGGC | | UUGUAGCC | | |
| hsa-miR- | CGGCGGGGACGGC | 30229 | GACCAAUCGCCGUC | 32271 | |
| 1908 | GAUUGGUC | | CCCGCCG | | |
| hsa-miR- | CGCAGGGGCCGGG | 30230 | CGGUGAGCACCCGG | 32272 | |
| 1909-3p | UGCUCACCG | | CCCCUGCG | | |
| hsa-miR- | UGAGUGCCGGUG | 3023 | CAGGGCAGGCACCG | 32273 | |
| 1909-5p | CCUGCCCUG | | GCACUCA | | |
| hsa-miR- | GCUGCGCUUGGA | 30232 | GGGGACGAAAUCCA | 32274 | |
| 191-3p | UUUCGUCCCC | | AGCGCAGC | | |
| hsa-miR- | CAACGGAAUCCCA | 30233 | CAGCUGCUUUUGGG | 32275 | |
| 191-5p | AAAGCAGCUG | | AUUCCGUUG | | |
| hsa-miR- | UCAGGCCAGGCAC | 30234 | UGAGCCACUGUGCC | 32276 | |
| 1972 | AGUGGCUCA | | UGGCCUGA | | |
| hsa-miR- | ACCGUGCAAAGG | 30235 | UAUGCUACCUUUGC | 32277 | |
| 1973 | UAGCAUA | | ACGGU | | |
| hsa-miR- | CCUCCUGCCCUCC | 30236 | ACAGCAAGGAGGGC | 32278 | |
| 1976 | UUGCUGU | | AGGAGG | | |
| hsa-miR- | UGUUUUGAUAAC | 30237 | ACAUUACUGUUAUC | 32279 | |
| 2052 | AGUAAUGU | | AAAACA | | |
| hsa-miR- | GUGUUAAUUAAA | 30238 | GUAAAUAGAGGUUU | 32280 | |
| 2053 | CCUCUAUUUAC | | AAUUAACAC | | |
| hsa-miR- | CUGUAAUAUAAA | 30239 | AAUAAAUUAAAUUU | 32281 | |
| 2054 | UUUAAUUUAUU | | AUAUUACAG | | |
| hsa-miR- | UUGGGGAAACGG | 30240 | CACUCAGCGGCCGU | 32282 | |
| 2110 | CCGCUGAGUG | | UUCCCCAA | | |
| hsa-miR- | CCUCCCAUGCCAA | 30241 | GGGAGUUCUUGGCA | 32283 | |
| 2116-3p | GAACUCCC | | UGGGAGG | | |
| hsa-miR- | GGUUCUUAGCAU | 30242 | AGACCUCCUAUGCU | 32284 | |
| 2116-5p | AGGAGGUCU | | AAGAACC | | |
| hsa-miR- | UGUUCUCUUUGCC | 30243 | CUGUCCUUGGCAAA | 32285 | |
| 2117 | AAGGACAG | | GAGAACA | | |
| hsa-miR- | AAAUCUCUGCAG | 30244 | UCACAUUUGCCUGC | 32286 | |
| 216b | GCAAAUGUGA | | AGAGAUUU | | |
| hsa-miR- | CAUGGUUCUGUC | 30245 | CGCGGUGCUUGACA | 32287 | |
| 218-2-3p | AAGCACCGCG | | GAACCAUG | | |
| hsa-miR- | UUGUGCUUGAUC | 30246 | ACAUGGUUAGAUCA | 32288 | |
| 218-5p | UAACCAUGU | | AGCACAA | | |
| hsa-miR- | UCUGCAAGUGUC | 30247 | CCUCGCCUCUGACA | 32289 | |
| 2276 | AGAGGCGAGG | | CUUGCAGA | | |
| hsa-miR- | GAGAGCAGUGUG | 30248 | CCAGGCAACACACA | 32290 | |
| 2278 | UGUUGCCUGG | | CUGCUCUC | | |
| hsa-miR- | AUCACAUUGCCAG | 30249 | GGGUAAUCACUGGC | 32291 | |
| 23c | UGAUUACCC | | AAUGUGAU | | |
| hsa-miR- | AGCAGAGGCAGA | 30250 | CCUGAGCCUCUCUG | 32292 | |
| 2467-3p | GAGGCUCAGG | | CCUCUGCU | | |
| hsa-miR- | UGAGGCUCUGUU | 30251 | GAGCCAAGGCUAAC | 32293 | |
| 2467-5p | AGCCUUGGCUC | | AGAGCCUCA | | |
| hsa-miR- | UAUCAUGGAGUU | 30252 | GUGCUUUACCAACU | 32294 | |
| 2681-3p | GGUAAAGCAC | | CCAUGAUA | | |
| hsa-miR- | GUUUUACCACCUC | 30253 | AGUCUCCUGGAGGU | 32295 | |
| 2681-5p | CAGGAGACU | | GGUAAAAC | | |
| hsa-miR- | CGCCUCUUCAGCG | 30254 | GGAAGACAGCGCUG | 32296 | |
| 2682-3p | CUGUCUUCC | | AAGAGGCG | | |
| hsa-miR- | CAGGCAGUGACU | 30255 | GACGUCUGAACAGU | 32297 | |
| 2682-5p | GUUCAGACGUC | | CACUGCCUG | | |
| hsa-miR- | AGAAUUGCGUUU | 30256 | ACUGAUUGUCCAAA | 32298 | |
| 2964a-3p | GGACAAUCAGU | | CGCAAUUCU | | |
| hsa-miR- | AGAUGUCCAGCCA | 30257 | CGAGAAUUGUGGCU | 32299 | |
| 2964a-5p | CAAUUCUCG | | GGACAUCU | | |
| hsa-miR- | AGCAGAAGCAGG | 30258 | UGGGAGAACCUCCC | 32300 | |
| 298 | GAGGUUCUCCCA | | UGCUUCUGCU | | |
| hsa-miR- | UAUGUGGGAUGG | 30259 | AAGCGGUUUACCAU | 32301 | |
| 299-3p | UAAACCGCUU | | CCCACAUA | | |
| hsa-miR- | UGGUUUACCGUCC | 30260 | AUGUAUGUGGGACG | 32302 | |
| 299-5p | CACAUACAU | | GUAAACCA | | |
| hsa-miR- | CAGUGCAAUGAU | 30261 | GCUUUGACAAUAUC | 32303 | |
| 301b | AUUGUCAAAGC | | AUUGCACUG | | |
| hsa-miR- | UAAUUGCUUCCA | 30262 | AAACAUGGAAGCAA | 32304 | |
| 302f | UGUUU | | UUA | | |
| hsa-miR- | UUGCCACACUGCA | 30263 | UGUAAGGUGUUGCA | 32305 | |
| 3064-3p | ACACCUUACA | | GUGUGGCAA | | |
| hsa-miR- | UCUGGCUGUUGU | 30264 | UUGCACACCACAAC | 32306 | |
| 3064-5p | GGUGUGCAA | | AGCCAGA | | |
| hsa-miR- | GAUAUCAGCUCA | 30265 | CGGUGCCUACUGAG | 32307 | |
| 3074-3p | GUAGGCACCG | | CUGAUAUC | | |
| hsa-miR- | GUUCCUGCUGAAC | 30266 | CUGGCUCAGUUCAG | 32308 | |
| 3074-5p | UGAGCCAG | | CAGGAAC | | |
| hsa-miR- | AUAUGGGUUUAC | 30267 | ACCAACUAGUAAAC | 32309 | |
| 3115 | UAGUUGGU | | CCAUAU | | |
| hsa-miR- | UGCUAUGCCAACA | 30268 | AUGGCAAUAUGUUG | 32310 | |
| 31-3p | UAUUGCCAU | | GCAUAGCA | | |
| hsa-miR- | AGGCAAGAUGCU | 30269 | AGCUAUGCCAGCAU | 32311 | |
| 31-5p | GGCAUAGCU | | CUUGCCU | | |
| hsa-miR- | CGGGGCGGCAGG | 30270 | GAGGCCCCUGOCGC | 32312 | |
| 3196 | GGCCUC | | CCCG | | |
| hsa-miR- | AAAAGCUGGGUU | 30271 | UCCUCUCAACCCAG | 32313 | |
| 320d | GAGAGGA | | CUUUU | | |
| hsa-miR- | CCCAAUACACGGU | 30272 | AAGAGGUCGACCGU | 32314 | |
| 323b-3p | CGACCUCUU | | GUAUUGGG | | |
| hsa-miR- | AGGUUGUCCGUG | 30273 | UGCGAACUCACCAC | 32315 | |
| 323b-5p | GUGAGUUCGCA | | GGACAACCU | | |
| hsa-miR- | GCAAAGCACACGG | 30274 | UCUCUGCAGGCCGU | 32316 | |
| 330-3p | CCUGCAGAGA | | GUGCUUUGC | | |
| hsa-miR- | UCUCUGGGCCUGU | 30275 | GCCUAAGACACAGG | 32317 | |
| 330-5p | GUCUUAGGC | | COCAGAGA | | |
| hsa-miR- | GCCCCUGGGCCUA | 30276 | UUCUAGGAUAGGCC | 32318 | |
| 331-3p | UCCUAGAA | | CAGGGGC | | |
| hsa-miR- | UCCGUCUCAGUUA | 30277 | GCUAUAAAGUAACU | 32319 | |
| 340-3p | CUUUAUAGC | | GAGACGGA | | |
| hsa-miR- | AACACACCUAUUC | 30278 | UGAAUCCUUGAAUA | 32320 | |
| 362-3p | AAGGAUUCA | | GGUGUGUU | | |
| hsa-miR- | AAUCCUUGGAACC | 30279 | ACUCACACCUAGGU | 32321 | |
| 362-5p | UAGGUGUGAGU | | UCCAAGGAUU | | |
| hsa-miR- | UAAUGCCCCUAAA | 30280 | AUAAGGAUUUUUAG | 32322 | |
| 365a-3p | AAUCCUUAU | | GGGCAUUA | | |
| hsa-miR- | AGGGACUUUUGG | 30281 | CACAUCUGCCCCCA | 32323 | |
| 365a-5p | GGGCAGAUGUG | | AAAGUCCCU | | |
| hsa-miR- | UAAUGCCCCUAAA | 30282 | AUAAGGAUUUUUAG | 32324 | |
| 365b-3p | AAUCCUUAU | | GGGCAUUA | | |
| hsa-miR- | AGGGACUUUCAG | 30283 | ACAGCUGCCCCUGA | 32325 | |
| 365b-5p | GGGCAGCUGU | | AAGUCCCU | | |
| hsa-miR- | GAAAAUGAUGAG | 30284 | CAUCAGUCACUACU | 32326 | |
| 3662 | UAGUGACUGAUG | | CAUCAUUUUC | | |
| hsa-miR- | UGAGCACCACACA | 30285 | GCGCCCGGCCUGUG | 32327 | |
| 3663-3p | GGCCGGGCGC | | UGGUGCUCA | | |
| hsa-miR- | GCUGGUCUGCGU | 30286 | CCGAGCACCACGCA | 32328 | |
| 3663-5p | GGUGCUCGG | | GACCAGC | | |
| hsa-miR- | GCCUGCUGGGGU | 30287 | ACCAGGUUCCACCC | 32329 | |
| 370 | GGAACCUGGU | | CAGCAGGC | | |
| hsa-miR- | GAAGUGCUUCGA | 30288 | ACACCCCAAAAUCG | 32330 | |
| 373-3p | UUUUGGGGUGU | | AAGCACUUC | | |
| hsa-miR- | ACUCAAAAUGGG | 30289 | GGAAAGCGCCCCCA | 32331 | |
| 373-5p | GGCGCUUUCC | | UUUUGAGU | | |
| hsa-miR- | UAUGUAACAUGG | 30290 | AGUUAGUGGACCAU | 32332 | |
| 379-3p | UCCACUAACU | | GUUACAUA | | |
| hsa-miR- | UGGUAGACUAUG | 30291 | CCUACGUUCCAUAG | 32333 | |
| 379-5p | GAACGUAGG | | UCUACCA | | |
| hsa-miR- | UGUAGAUACGAG | 30292 | GUGGCUGGUGCUCG | 32334 | |
| 3935 | CACCAGCCAC | | UAUCUACA | | |
| hsa-miR- | ACAGGCGGCUGU | 30293 | CCCCCAUUGCUACA | 32335 | |
| 3937 | AGCAAUGGGGG | | GCCGCCUGU | | |
| hsa-miR- | AAUUCCCUUGUA | 30294 | CCGGGUUAUCUACA | 32336 | |
| 3938 | GAUAACCCGG | | AGGGAAUU | | |
| hsa-miR- | UACGOGCAGACCA | 30295 | GACAUCCUGUGGUC | 32337 | |
| 3939 | CAGGAUGUC | | UGCGCGUA | | |
| hsa-miR- | UUACACACAACUG | 30296 | UAUGAUCCUCAGUU | 32338 | |
| 3941 | AGGAUCAUA | | GUGUGUAA | | |
| hsa-miR- | UAGCCCCCAGGCU | 30297 | CGCCAAGUGAAGCC | 32339 | |
| 3943 | UCACUUGGCG | | UGGGGGCUA | | |
| hsa-miR- | AGGGCAUAGGAG | 30298 | AUAUCAACCCUCUC | 32340 | |
| 3945 | AGGGUUGAUAU | | CUAUGCCCU | | |
| hsa-miR- | GAAUGUUGCUCG | 30299 | AGGGGUUCACCGAG | 32341 | |
| 409-3p | GUGAACCCCU | | CAACAUUC | | |
| hsa-miR- | AGGUUACCCGAGC | 30300 | AUGCAAAGUUGCUC | 32342 | |
| 409-5p | AACUUUGCAU | | GGGUAACCU | | |
| hsa-miR- | UAUGUAACACGG | 30301 | GGUUAGUGGACCGU | 32343 | |
| 411-3p | UCCACUAACC | | GUUACAUA | | |
| hsa-miR- | UAGUAGACCGUA | 30302 | CGUACGCUAUACGG | 32344 | |
| 411-5p | UAGCGUACG | | UCUACUA | | |
| hsa-miR- | ACUUCACCUGGUC | 30303 | ACGGCUAGUGGACC | 32345 | |
| 412 | CACUAGCCGU | | AGGUGAAGU | | |
| hsa-miR- | GUGUUCUCUGAU | 30304 | CUGUCCAUCAGAGA | 32346 | |
| 4273 | GGACAG | | ACAC | | |
| hsa-miR- | CAGGUCGUCUUGC | 30305 | AGAAGCCCUGCAAG | 32347 | |
| 431-3p | AGGGCUUCU | | ACGACCUG | | |
| hsa-miR- | UGUCUUGCAGGCC | 30306 | UGCAUGACGGCCUG | 32348 | |
| 431-5p | GUCAUGCA | | CAAGACA | | |
| hsa-miR- | AUCAUGAUGGGC | 30307 | ACACCGAGGAGCCC | 32349 | |
| 433 | UCCUCGGUGU | | AUCAUGAU | | |
| hsa-miR- | UUGCUAGUUGCA | 30308 | ACAGAGAGGAGUGC | 32350 | |
| 449c-3p | CUCCUCUCUGU | | AACUAGCAA | | |
| hsa-miR- | UAGGCAGUGUAU | 30309 | ACAGCCGCUAGCAA | 32351 | |
| 449c-5p | UGCUAGCGGCUG | | UACACUGCCUA | | |
| | U | | | | |
| hsa-miR- | AUUGGGGACAUU | 30310 | AUGAAUGCAAAAUG | 32352 | |
| 450a-3p | UUGCAUUCAU | | UCCCCAAU | | |
| hsa-miR- | UUUUGCGAUGUG | 30311 | AUAUUAGGAACACA | 32353 | |
| 450a-5p | UUCCUAAUAU | | UCGCAAAA | | |
| hsa-miR- | UUGGGAUCAUUU | 30312 | UAUGGAUGCAAAAU | 32354 | |
| 450b-3p | UGCAUCCAUA | | GAUCCCAA | | |
| hsa-miR- | UUUUGCAAUAUG | 30313 | UAUUCAGGAACAUA | 32355 | |
| 450b-5p | UUCCUGAAUA | | UUGCAAAA | | |
| hsa-miR- | GCAGUCCAUGGGC | 30314 | GUGUAUAUGCCCAU | 32356 | |
| 455-3p | AUAUACAC | | GGACUGC | | |
| hsa-miR- | UAUGUGCCUUUG | 30315 | CGAUGUAGUCCAAA | 32357 | |
| 455-5p | GACUACAUCG | | GGCACAUA | | |
| hsa-miR- | AUACACAUACACG | 30316 | AUGUGUGUUGCGUG | 32358 | |
| 466 | CAACACACAU | | UAUGUGUAU | | |
| hsa-miR- | UUGCAUGUCAGA | 30317 | GGGAAUUACAAUCU | 32359 | |
| 4666b | UUGUAAUUCCC | | GACAUGCAA | | |
| hsa-miR- | UCACUCCUCUCCU | 30318 | AAGACGGGAGGAGA | 32360 | |
| 483-3p | CCCGUCUU | | GGAGUGA | | |
| hsa-miR- | UCAGGCUCAGUCC | 30319 | AUCGGGAGGGGACU | 32361 | |
| 484 | CCUCCCGAU | | GAGCCUGA | | |
| hsa-miR- | GUCAUACACGGCU | 30320 | AGAGAGGAGAGCCG | 32362 | |
| 485-3p | CUCCUCUCU | | UGUAUGAC | | |
| hsa-miR- | AGAGGCUGGCCG | 30321 | GAAUUCAUCACGGC | 32363 | |
| 485-5p | UGAUGAAUUC | | CAGCCUCU | | |
| hsa-miR- | AAUCAUACAGGG | 30322 | AACUGGAUGUCCCU | 32364 | |
| 487a | ACAUCCAGUU | | GUAUGAUU | | |
| hsa-miR- | AAUCGUACAGGG | 30323 | AAGUGGAUGACCCU | 32365 | |
| 487b | UCAUCCACUU | | GUACGAUU | | |
| hsa-miR- | UUGAAAGGCUAU | 30324 | GACCAAGAAAUAGC | 32366 | |
| 488-3p | UUCUUGGUC | | CUUUCAA | | |
| hsa-miR- | CCCAGAUAAUGGC | 30325 | UUGAGAGUGCCAUU | 32367 | |
| 488-5p | ACUCUCAA | | AUCUGGG | | |
| hsa-miR- | CAACCUGGAGGAC | 30326 | CAGCAUGGAGUCCU | 32368 | |
| 490-3p | UCCAUGCUG | | CCAGGUUG | | |
| hsa-miR- | CCAUGGAUCUCCA | 30327 | ACCCACCUGGAGAU | 32369 | |
| 490-5p | GGUGGGU | | CCAUGG | | |
| hsa-miR- | CUUAUGCAAGAU | 30328 | GUAGAAGGGAAUCU | 32370 | |
| 491-3p | UCCCUUCUAC | | UGCAUAAG | | |
| hsa-miR- | AGUGGGGAACCC | 30329 | CCUCAUGGAAGGGU | 32371 | |
| 491-5p | UUCCAUGAGG | | UCCCCACU | | |
| hsa-miR- | AGGACCUGCGGG | 30330 | AAGAAUCUUGUCCC | 32372 | |
| 492 | ACAAGAUUCUU | | GCAGGUCCU | | |
| hsa-miR- | CAAACCACACUGU | 30331 | UCUAACACCACAGU | 32373 | |
| 497-3p | GGUGUUAGA | | GUGGUUUG | | |
| hsa-miR- | CAGCAGCACACUG | 30332 | ACAAACCACAGUGU | 32374 | |
| 497-5p | UGGUUUGU | | GCUGCUG | | |
| hsa-miR- | UUUCAAGCCAGG | 30333 | GAAAAACGCCCCCU | 32375 | |
| 498 | GGGCGUUUUUC | | GGCUUGAAA | | |
| hsa-miR- | UCACUACCUGACA | 30334 | ACUGUAUUGUCAGG | 32376 | |
| 4999-3p | AUACAGU | | UAGUGA | | |
| hsa-miR- | UGCUGUAUUGUC | 30335 | UCACUACCUGACAA | 32377 | |
| 4999-5p | AGGUAGUGA | | UACAGCA | | |
| hsa-miR- | UUCUGCCUCUGUC | 30336 | AAGGACCUGGACAG | 32378 | |
| 5001-3p | CAGGUCCUU | | AGGCAGAA | | |
| hsa-miR- | AGGGCUGGACUC | 30337 | AGCUCCGCCGCUGA | 32379 | |
| 5001-5p | AGCGGCGGAGCU | | GUCCAGCCCU | | |
| hsa-miR- | UGACUGCCUCACU | 30338 | AAGUGGUCAGUGAG | 32380 | |
| 5002-3p | GACCACUU | | GCAGUCA | | |
| hsa-miR- | AAUUUGGUUUCU | 30339 | ACUAAGUGCCUCAG | 32381 | |
| 5002-5p | GAGGCACUUAGU | | AAACCAAAUU | | |
| hsa-miR- | UACUUUUCUAGG | 30340 | CCCCAACAACCUAG | 32382 | |
| 5003-3p | UUGUUGGGG | | AAAAGUA | | |
| hsa-miR- | UCACAACAACCUU | 30341 | UCUACCCUGCAAGG | 32383 | |
| 5003-5p | GCAGGGUAGA | | UUGUUGUGA | | |
| hsa-miR- | CUUGGAUUUUCC | 30342 | CUGAGGCCCAGGAA | 32384 | |
| 5004-3p | UGGGCCUCAG | | AAUCCAAG | | |
| hsa-miR- | UGAGGACAGGGC | 30343 | UCGUGAAUUUGCCC | 32385 | |
| 5004-5p | AAAUUCACGA | | UGUCCUCA | | |
| hsa-miR- | AUCAUAUGAACC | 30344 | AUUAGAGUUUGGUU | 32386 | |
| 5007-3p | AAACUCUAAU | | CAUAUGAU | | |
| hsa-miR- | UAGAGUCUGGCU | 30345 | AAACCAUAUCAGCC | 32387 | |
| 5007-5p | GAUAUGGUUU | | AGACUCUA | | |
| hsa-miR- | CCUGUGCUCCCAG | 30346 | GCGAGGCCCUGGGA | 32388 | |
| 5008-3p | GGCCUCGC | | GCACAGG | | |
| hsa-miR- | UGAGGCCCUUGG | 30347 | CCACUGUGCCCCAA | 32389 | |
| 5008-5p | GGCACAGUGG | | GGGCCUCA | | |
| hsa-miR- | UCCUAAAUCUGA | 30348 | UUUUGGACUUUCAG | 32390 | |
| 5009-3p | AAGUCCAAAA | | AUUUAGGA | | |
| hsa-miR- | UUGGACUUUUUC | 30349 | AUCCCCAAAUCUGA | 32391 | |
| 5009-5p | AGAUUUGGGGAU | | AAAAGUCCAA | | |
| hsa-miR- | AUGCACCUGGGCA | 30350 | CAGAAUCCUUGCCC | 32392 | |
| 500a-3p | AGGAUUCUG | | AGGUGCAU | | |
| hsa-miR- | UAAUCCUUGCUAC | 30351 | UCUCACCCAGGUAG | 32393 | |
| 500a-5p | CUGGGUGAGA | | CAAGGAUUA | | |
| hsa-miR- | UUUUGUGUCUCCC | 30352 | CUGGGGAAUGGGAG | 32394 | |
| 5010-3p | AUUCCCCAG | | ACACAAAA | | |
| hsa-miR- | AGGGGGAUGGCA | 30353 | AAUUUUGCUCUGCC | 32395 | |
| 5010-5p | GAGCAAAAUU | | AUCCCCCU | | |
| hsa-miR- | GUGCAUGGCUGU | 30354 | UGUUAUAUAUACAG | 32396 | |
| 5011-3p | AUAUAUAACA | | CCAUGCAC | | |
| hsa-miR- | UAUAUAUACAGC | 30355 | GAGUGCAUGGCUGU | 32397 | |
| 5011-5p | CAUGCACUC | | AUAUAUA | | |
| hsa-miR- | AAUGCACCCGGGC | 30356 | AGAAUCCUUGCCCG | 32398 | |
| 501-3p | AAGGAUUCU | | GGUGCAUU | | |
| hsa-miR- | AAUCCUUUGUCCC | 30357 | UCUCACCCAGGGAC | 32399 | |
| 501-5p | UGGGUGAGA | | AAAGGAUU | | |
| hsa-miR- | AAUGCACCUGGGC | 30358 | UGAAUCCUUGCCCA | 32400 | |
| 502-3p | AAGGAUUCA | | GGUGCAUU | | |
| hsa-miR- | AUCCUUGCUAUCU | 30359 | UAGCACCCAGAUAG | 32401 | |
| 502-5p | GGGUGCUA | | CAAGGAU | | |
| hsa-miR- | AGACCCUGGUCUG | 30360 | GAUAGAGUGCAGAC | 32402 | |
| 504 | CACUCUAUC | | CAGGGUCU | | |
| hsa-miR- | UUGCAGCUGCGG | 30361 | ACCUUACAACCGCA | 32403 | |
| 5047 | UUGUAAGGU | | GCUGCAA | | |
| hsa-miR- | CGUCAACACUUGC | 30362 | AGGAAACCAGCAAG | 32404 | |
| 505-3p | UGGUUUCCU | | UGUUGACG | | |
| hsa-miR- | GGGAGCCAGGAA | 30363 | ACAUCAAUACUUCC | 32405 | |
| 505-5p | GUAUUGAUGU | | UGGCUCCC | | |
| hsa-miR- | UAAGGCACCCUUC | 30364 | UCUACUCAGAAGGG | 32406 | |
| 506-3p | UGAGUAGA | | UGCCUUA | | |
| hsa-miR- | UAUUCAGGAAGG | 30365 | UUAAGUAACACCUU | 32407 | |
| 506-5p | UGUUACUUAA | | CCUGAAUA | | |
| hsa-miR- | UUUUGCACCUUU | 30366 | UUCACUCCAAAAGG | 32408 | |
| 507 | UGGAGUGAA | | UGCAAAA | | |
| hsa-miR- | UGAUUGUAGCCU | 30367 | UCUACUCCAAAAGG | 32409 | |
| 508-3p | UUUGGAGUAGA | | CUACAAUCA | | |
| hsa-miR- | GGGUUUGUAGCU | 30368 | CAUGCCAGCAAAGC | 32410 | |
| 5087 | UUGCUGGCAUG | | UACAAACCC | | |
| hsa-miR- | CAGGGCUCAGGG | 30369 | CUCCAUCCAAUCCC | 32411 | |
| 5088 | AUUGGAUGGAG | | UGAGCCCUG | | |
| hsa-miR- | AUGCUACUCGGA | 30370 | UCAGUGGGAUUUCC | 32412 | |
| 5089-3p | AAUCCCACUGA | | GAGUAGCAU | | |
| hsa-miR- | GUGGGAUUUCUG | 30371 | GAUGCUACUCAGAA | 32413 | |
| 5089-5p | AGUAGCAUC | | AUCCCAC | | |
| hsa-miR- | CCGGGGCAGAUU | 30372 | CACCCUACACCAAU | 32414 | |
| 5090 | GGUGUAGGGUG | | CUGCCCCGG | | |
| hsa-miR- | ACGGAGACGACA | 30373 | CAGCACAGUCUUGU | 32415 | |
| 5091 | AGACUGUGCUG | | CGUCUCCGU | | |
| hsa-miR- | AAUCCACGCUGAG | 30374 | GAUGCCAAGCUCAG | 32416 | |
| 5092 | CUUGGCAUC | | CGUGGAUU | | |
| hsa-miR- | AGGAAAUGAGGC | 30375 | GCUCCUAGCCAGCC | 32417 | |
| 5093 | UGGCUAGGAGC | | UCAUUUCCU | | |
| hsa-miR- | UGAUUGGUACGU | 30376 | CUACCCACAGACGU | 32418 | |
| 509-3p | CUGUGGGUAG | | ACCAAUCA | | |
| hsa-miR- | AAUCAGUGAAUG | 30377 | AGGUUCAAGGCAUU | 32419 | |
| 5094 | CCUUGAACCU | | CACUGAUU | | |
| hsa-miR- | UUACAGGCGUGA | 30378 | CGCGGUGGUUCACG | 32420 | |
| 5095 | ACCACCGCG | | CCUGUAA | | |
| hsa-miR- | UACUGCAGACAG | 30379 | UGAUUGCCACUGUC | 32421 | |
| 509-5p | UGGCAAUCA | | UGCAGUA | | |
| hsa-miR- | GUUUCACCAUGU | 30380 | GCCUGACCAACAUG | 32422 | |
| 5096 | UGGUCAGGC | | GUGAAAC | | |
| hsa-miR- | UAAAUUUCACCU | 30381 | CCUUCUCAGAAAGG | 32423 | |
| 513a-3p | UUCUGAGAAGG | | UGAAAUUUA | | |
| hsa-miR- | UUCACAAGGAGG | 30382 | AUAAAUGACACCUC | 32424 | |
| 513b | UGUCAUUUAU | | CUUGUGAA | | |
| hsa-miR- | UAAAUUUCACCU | 30383 | UCUUCUCAGAAAGG | 32425 | |
| 513c-3p | UUCUGAGAAGA | | UGAAAUUUA | | |
| hsa-miR- | UUCUCAAGGAGG | 30384 | AUAAACGACACCUC | 32426 | |
| 513c-5p | UGUCGUUUAU | | CUUGAGAA | | |
| hsa-miR- | AUUGACACUUCU | 30385 | UCUACUCACAGAAG | 32427 | |
| 514a-3p | GUGAGUAGA | | UGUCAAU | | |
| hsa-miR- | UACUCUGGAGAG | 30386 | CAUGAUUGUCACUC | 32428 | |
| 514a-5p | UGACAAUCAUG | | UCCAGAGUA | | |
| hsa-miR- | AUUGACACCUCUG | 30387 | UCCACUCACAGAGG | 32429 | |
| 514b-3p | UGAGUGGA | | UGUCAAU | | |
| hsa-miR- | UUCUCAAGAGGG | 30388 | AUGAUUGCCUCCCU | 32430 | |
| 514b-5p | AGGCAAUCAU | | CUUGAGAA | | |
| hsa-miR- | GAGUGCCUUCUU | 30389 | AACGCUCCAAAAGA | 32431 | |
| 515-3p | UUGGAGCGUU | | AGGCACUC | | |
| hsa-miR- | UGCUUCCUUUCAG | 30390 | ACCCUCUGAAAGGA | 32432 | |
| 516b-3p | AGGGU | | AGCA | | |
| hsa-miR- | AUCUGGAGGUAA | 30391 | AAAGUGCUUCUUAC | 32433 | |
| 516b-5p | GAAGCACUUU | | CUCCAGAU | | |
| hsa-miR- | GAAAGCGCUUCCC | 30392 | UCCAGCAAAGGGAA | 32434 | |
| 518a-3p | UUUGCUGGA | | GCGCUUUC | | |
| hsa-miR- | CUGCAAAGGGAA | 30393 | GAAAGGGCUUCCCU | 32435 | |
| 518a-5p | GCCCUUUC | | UUGCAG | | |
| hsa-miR- | CAAAGCGCUUCCC | 30394 | GCUCCAAAGGGAAG | 32436 | |
| 518d-3p | UUUGGAGC | | CGCUUUG | | |
| hsa-miR- | CUCUAGAGGGAA | 30395 | CAGAAAGUGCUUCC | 32437 | |
| 518d-5p | GCACUUUCUG | | CUCUAGAG | | |
| hsa-miR- | AAAGCGCUUCCCU | 30396 | CACUCUGAAGGGAA | 32438 | |
| 518e-3p | UCAGAGUG | | GCGCUUU | | |
| hsa-miR- | CUCUAGAGGGAA | 30397 | CAGAAAGCGCUUCC | 32439 | |
| 518e-5p | GCGCUUUCUG | | CUCUAGAG | | |
| hsa-miR- | AAAGUGCAUCCU | 30398 | AACCUCUAAAAGGA | 32440 | |
| 519b-3p | UUUAGAGGUU | | UGCACUUU | | |
| hsa-miR- | CUCUAGAGGGAA | 30399 | CAGAAAGCGCUUCC | 32441 | |
| 519b-5p | GOGCUUUCUG | | CUCUAGAG | | |
| hsa-miR- | AAAGUGCAUCUU | 30400 | AUCCUCUAAAAAGA | 32442 | |
| 519c-3p | UUUAGAGGAU | | UGCACUUU | | |
| hsa-miR- | CUCUAGAGGGAA | 30401 | CAGAAAGCGCUUCC | 32443 | |
| 519c-5p | GCGCUUUCUG | | CUCUAGAG | | |
| hsa-miR- | AAAGUGCUUCCU | 30402 | CCCUCUAAAAGGAA | 32444 | |
| 520b | UUUAGAGGG | | GCACUUU | | |
| hsa-miR- | AAAGUGCUUCCU | 30403 | ACCCUCUAAAAGGA | 32445 | |
| 520c-3p | UUUAGAGGGU | | AGCACUUU | | |
| hsa-miR- | CUCUAGAGGGAA | 30404 | CAGAAAGUGCUUCC | 32446 | |
| 520c-5p | GCACUUUCUG | | CUCUAGAG | | |
| hsa-miR- | AAAGUGCUUCUC | 30405 | ACCCACCAAAGAGA | 32447 | |
| 520d-3p | UUUGGUGGGU | | AGCACUUU | | |
| hsa-miR- | CUACAAAGGGAA | 30406 | GAAAGGGCUUCCCU | 32448 | |
| 520d-5p | GCCCUUUC | | UUGUAG | | |
| hsa-miR- | AAAGUGCUUCCU | 30407 | CCCUCAAAAAGGAA | 32449 | |
| 520e | UUUUGAGGG | | GCACUUU | | |
| hsa-miR- | AAGUGCUUCCUU | 30408 | AACCCUCUAAAAGG | 32450 | |
| 520f | UUAGAGGGUU | | AAGCACUU | | |
| hsa-miR- | ACAAAGUGCUUCC | 30409 | ACACUCUAAAGGGA | 32451 | |
| 520g | CUUUAGAGUGU | | AGCACUUUGU | | |
| hsa-miR- | AACGCACUUCCCU | 30410 | ACACUCUAAAGGGA | 32452 | |
| 521 | UUAGAGUGU | | AGUGCGUU | | |
| hsa-miR- | AAAAUGGUUCCC | 30411 | ACACUCUAAAGGGA | 32453 | |
| 522-3p | UUUAGAGUGU | | ACCAUUUU | | |
| hsa-miR- | CUCUAGAGGGAA | 30412 | CAGAAAGCGCUUCC | 32454 | |
| 522-5p | GCGCUUUCUG | | CUCUAGAG | | |
| hsa-miR- | GAACGCGCUUCCC | 30413 | ACCCUCUAUAGGGA | 32455 | |
| 523-3p | UAUAGAGGGU | | AGCGCGUUC | | |
| hsa-miR- | CUCUAGAGGGAA | 30414 | CAGAAAGCGCUUCC | 32456 | |
| 523-5p | GCGCUUUCUG | | CUCUAGAG | | |
| hsa-miR- | GAAGGCGCUUCCC | 30415 | ACUCCAAAGGGAAG | 32457 | |
| 524-3p | UUUGGAGU | | CGCCUUC | | |
| hsa-miR- | CUGCAAAGGGAA | 30416 | GAAAGGGCUUCCCU | 32458 | |
| 527 | GCCCUUUC | | UUGCAG | | |
| hsa-miR- | CCUCCCACACCCA | 30417 | UGCAAGCCUUGGGU | 32459 | |
| 532-3p | AGGCUUGCA | | GUGGGAGG | | |
| hsa-miR- | CAUGCCUUGAGU | 30418 | ACGGUCCUACACUC | 32460 | |
| 532-5p | GUAGGACCGU | | AAGGCAUG | | |
| hsa-miR- | AUCAUACAAGGA | 30419 | AAAGAAAUUGUCCU | 32461 | |
| 539-3p | CAAUUUCUUU | | UGUAUGAU | | |
| hsa-miR- | GGAGAAAUUAUC | 30420 | ACACACCAAGGAUA | 32462 | |
| 539-5p | CUUGGUGUGU | | AUUUCUCC | | |
| hsa-miR- | UGGUGGGCACAG | 30421 | AGUCCAGAUUCUGU | 32463 | |
| 541-3p | AAUCUGGACU | | GCCCACCA | | |
| hsa-miR- | AAAGGAUUCUGC | 30422 | AGUGGGACCGACAG | 32464 | |
| 541-5p | UGUCGGUCCCACU | | CAGAAUCCUUU | | |
| hsa-miR- | UCGGGGAUCAUC | 30423 | UCUCGUGACAUGAU | 32465 | |
| 542-5p | AUGUCACGAGA | | GAUCCCCGA | | |
| hsa-miR- | AAACAUUCGCGG | 30424 | AAGAAGUGCACCGC | 32466 | |
| 543 | UGCACUUCUU | | GAAUGUUU | | |
| hsa-miR- | AUUCUGCAUUUU | 30425 | GAACUUGCUAAAAA | 32467 | |
| 544a | UAGCAAGUUC | | UGCAGAAU | | |
| hsa-miR- | ACCUGAGGUUGU | 30426 | UUAGAAAUGCACAA | 32468 | |
| 544b | GCAUUUCUAA | | CCUCAGGU | | |
| hsa-miR- | UCAGCAAACAUU | 30427 | GCACACAAUAAAUG | 32469 | |
| 545-3p | UAUUGUGUGC | | UUUGCUGA | | |
| hsa-miR- | UCAGUAAAUGUU | 30428 | UCAUCUAAUAAACA | 32470 | |
| 545-5p | UAUUAGAUGA | | UUUACUGA | | |
| hsa-miR- | AGCUACAGUUAC | 30429 | UGGUGCAAAAGUAA | 32471 | |
| 548 | UUUUGCACCA | | CUGUAGCU | | |
| hsa-miR- | UAAAAACUGCAA | 30430 | GAAAGUAAUUGCAG | 32472 | |
| 548-3p | UUACUUUC | | UUUUUA | | |
| hsa-miR- | UGCAAAAGUAAU | 30431 | CAAAAACUGCAAUU | 32473 | |
| 548-5p | UGCAGUUUUUG | | ACUUUUGCA | | |
| hsa-miR- | AAAGACCGUGAC | 30432 | UGCAAAAGUAGUCA | 32474 | |
| 548ao-3p | UACUUUUGCA | | CGGUCUUU | | |
| hsa-miR- | AGAAGUAACUAC | 30433 | UGCAAAAACCGUAG | 32475 | |
| 548ao-5p | GGUUUUUGCA | | UUACUUCU | | |
| hsa-miR- | AAAAACCACAAU | 30434 | AAAAGUAAUUGUGG | 32476 | |
| 548ap-3p | UACUUUU | | UUUUU | | |
| hsa-miR- | AAAAGUAAUUGC | 30435 | AAAGACCGCAAUUA | 32477 | |
| 548ap-5p | GGUCUUU | | CUUUU | | |
| hsa-miR- | CAAAAACUGCAA | 30436 | GCAAAAGUAAUUGC | 32478 | |
| 548ag-3p | UUACUUUUGC | | AGUUUUUG | | |
| hsa-miR- | GAAAGUAAUUGC | 30437 | GGCAAAAACAGCAA | 32479 | |
| 548aq-5p | UGUUUUUGCC | | UUACUUUC | | |
| hsa-miR- | UAAAACUGCAGU | 30438 | GCAAAAAUAACUGC | 32480 | |
| 548ar-3p | UAUUUUUGC | | AGUUUUA | | |
| hsa-miR- | AAAAGUAAUUGC | 30439 | GCAAAAACUGCAAU | 32481 | |
| 548ar-5p | AGUUUUUGC | | UACUUUU | | |
| hsa-miR- | UAAAACCCACAAU | 30440 | ACAAACAUAAUUGU | 32482 | |
| 548as-3p | UAUGUUUGU | | GGGUUUUA | | |
| hsa-miR- | AAAAGUAAUUGC | 30441 | GGCAAAACCCGCAA | 32483 | |
| 548as-5p | GGGUUUUGCC | | UUACUUUU | | |
| hsa-miR- | CAAAACCGCAGUA | 30442 | ACAAAAGUUACUGC | 32484 | |
| 548at-3p | ACUUUUGU | | GGUUUUG | | |
| hsa-miR- | AAAAGUUAUUGC | 30443 | AGCCAAAACCGCAA | 32485 | |
| 548at-5p | GGUUUUGGCU | | UAACUUUU | | |
| hsa-miR- | UGGCAGUUACUU | 30444 | CUGGUGCAAAAGUA | 32486 | |
| 548au-3p | UUGCACCAG | | ACUGCCA | | |
| hsa-miR- | AAAAGUAAUUGC | 30445 | GCAAAAACCGCAAU | 32487 | |
| 548au-5p | GGUUUUUGC | | UACUUUU | | |
| hsa-miR- | AAAACUGCAGUU | 30446 | GCAAAAGUAACUGC | 32488 | |
| 548av-3p | ACUUUUGC | | AGUUUU | | |
| hsa-miR- | AAAAGUACUUGC | 30447 | AAAUCCGCAAGUAC | 32489 | |
| 548av-5p | GGAUUU | | UUUU | | |
| hsa-miR- | GUGCAAAAGUCA | 30448 | AACCGUGAUGACUU | 32490 | |
| 548aw | UCACGGUU | | UUGCAC | | |
| hsa-miR- | GCUGGUGCAAAA | 30449 | CCGCCAUUACUUUU | 32491 | |
| 548q | GUAAUGGCGG | | GCACCAGC | | |
| hsa-miR- | AAAAGUAAUCAC | 30450 | GGCAAAAACAGUGA | 32492 | |
| 548y | UGUUUUUGCC | | UUACUUUU | | |
| hsa-miR- | AGUGCCUGAGGG | 30451 | CUCUUACUCCCUCA | 32493 | |
| 550a-3-5p | AGUAAGAG | | GGCACU | | |
| hsa-miR- | UGUCUUACUCCCU | 30452 | AUGUGCCUGAGGGA | 32494 | |
| 550a-3p | CAGGCACAU | | GUAAGACA | | |
| hsa-miR- | AGUGCCUGAGGG | 30453 | GGGCUCUUACUCCC | 32495 | |
| 550a-5p | AGUAAGAGCCC | | UCAGGCACU | | |
| hsa-miR- | GCGACCCACUCUU | 30454 | UGGAAACCAAGAGU | 32496 | |
| 55la | GGUUUCCA | | GGGUCGC | | |
| hsa-miR- | UUAGCUUAAGGA | 30455 | GAUCUGGUACUCCU | 32497 | |
| 5579-3p | GUACCAGAUC | | UAAGCUAA | | |
| hsa-miR- | UAUGGUACUCCU | 30456 | GUUAGCUUAAGGAG | 32498 | |
| 5579-5p | UAAGCUAAC | | UACCAUA | | |
| hsa-miR- | UGAGCUGCUGUA | 30457 | AUUUUGGUACAGCA | 32499 | |
| 558 | CCAAAAU | | GCUCA | | |
| hsa-miR- | CACAUAUGAAGU | 30458 | GUGCUGGCUCACUU | 32500 | |
| 5580-3p | GAGCCAGCAC | | CAUAUGUG | | |
| hsa-miR- | UGCUGGCUCAUU | 30459 | ACACAUAUGAAAUG | 32501 | |
| 5580-5p | UCAUAUGUGU | | AGCCAGCA | | |
| hsa-miR- | UUCCAUGCCUCCU | 30460 | GGAACUUCUAGGAG | 32502 | |
| 5581-3p | AGAAGUUCC | | GCAUGGAA | | |
| hsa-miR- | AGCCUUCCAGGAG | 30461 | UCUCCAUUUCUCCU | 32503 | |
| 5581-5p | AAAUGGAGA | | GGAAGGCU | | |
| hsa-miR- | UAAAACUUUAAG | 30462 | CCUAGGCACACUUA | 32504 | |
| 5582-3p | UGUGCCUAGG | | AAGUUUUA | | |
| hsa-miR- | UAGGCACACUUA | 30463 | GCUAUAACUUUAAG | 32505 | |
| 5582-5p | AAGUUAUAGC | | UGUGCCUA | | |
| hsa-miR- | GAAUAUGGGUAU | 30464 | CCAAACUAAUAUAC | 32506 | |
| 5583-3p | AUUAGUUUGG | | CCAUAUUC | | |
| hsa-miR- | AAACUAAUAUAC | 30465 | CAGAAUAUGGGUAU | 32507 | |
| 5583-5p | CCAUAUUCUG | | AUUAGUUU | | |
| hsa-miR- | UAGUUCUUCCCUU | 30466 | AAUUGGGCAAAGGG | 32508 | |
| 5584-3p | UGCCCAAUU | | AAGAACUA | | |
| hsa-miR- | CAGGGAAAUGGG | 30467 | UCUAGUUCUUCCCA | 32509 | |
| 5584-5p | AAGAACUAGA | | UUUCCCUG | | |
| hsa-miR- | CUGAAUAGCUGG | 30468 | ACCUGUAGUCCCAG | 32510 | |
| 5585-3p | GACUACAGGU | | CUAUUCAG | | |
| hsa-miR- | UGAAGUACCAGC | 30469 | CUCUCGAGUAGCUG | 32511 | |
| 5585-5p | UACUCGAGAG | | GUACUUCA | | |
| hsa-miR- | CAGAGUGACAAG | 30470 | CUUUAACCAGCUUG | 32512 | |
| 5586-3p | CUGGUUAAAG | | UCACUCUG | | |
| hsa-miR- | UAUCCAGCUUGU | 30471 | GCAUAUAGUAACAA | 32513 | |
| 5586-5p | UACUAUAUGC | | GCUGGAUA | | |
| hsa-miR- | GCCCCGGGCAGUG | 30472 | GAUGAUCACACUGC | 32514 | |
| 5587-3p | UGAUCAUC | | CCGGGGC | | |
| hsa-miR- | AUGGUCACCUCCG | 30473 | AGUCCCGGAGGUGA | 32515 | |
| 5587-5p | GGACU | | CCAU | | |
| hsa-miR- | AAGUCCCACUAAU | 30474 | GCUGGCAUUAGUGG | 32516 | |
| 5588-3p | GCCAGC | | GACUU | | |
| hsa-miR- | ACUGGCAUUAGU | 30475 | AAAAGUCCCACUAA | 32517 | |
| 5588-5p | GGGACUUUU | | UGCCAGU | | |
| hsa-miR- | UGCACAUGGCAAC | 30476 | UGGGAGCUAGGUUG | 32518 | |
| 5589-3p | CUAGCUCCCA | | CCAUGUGCA | | |
| hsa-miR- | GGCUGGGUGCUC | 30477 | ACUGCACAAGAGCA | 32519 | |
| 5589-5p | UUGUGCAGU | | CCCAGCC | | |
| hsa-miR- | UAAAGUAAAUAU | 30478 | UUUUGGUGCAUAUU | 32520 | |
| 559 | GCACCAAAA | | UACUUUA | | |
| hsa-miR- | AAUAAAGUUCAU | 30479 | UUGCCAUACAUGAA | 32521 | |
| 5590-3p | GUAUGGCAA | | CUUUAUU | | |
| hsa-miR- | UUGCCAUACAUA | 30480 | AAUAAAGUCUAUGU | 32522 | |
| 5590-5p | GACUUUAUU | | AUGGCAA | | |
| hsa-miR- | AUACCCAUAGCUU | 30481 | UGGGAGCUAAGCUA | 32523 | |
| 5591-3p | AGCUCCCA | | UGGGUAU | | |
| hsa-miR- | UGGGAGCUAAGC | 30482 | AUACCCAUAGCUUA | 32524 | |
| 5591-5p | UAUGGGUAU | | GCUCCCA | | |
| hsa-miR- | CAAAGUUUAAGA | 30483 | ACUUCAAGGAUCUU | 32525 | |
| 561-3p | UCCUUGAAGU | | AAACUUUG | | |
| hsa-miR- | AUCAAGGAUCUU | 30484 | GGCAAAGUUUAAGA | 32526 | |
| 561-5p | AAACUUUGCC | | UCCUUGAU | | |
| hsa-miR- | AAAGUAGCUGUA | 30485 | GCAAAUGGUACAGC | 32527 | |
| 562 | CCAUUUGC | | UACUUU | | |
| hsa-miR- | AGGCACGGUGUC | 30486 | GCCUGCUGACACCG | 32528 | |
| 564 | AGCAGGC | | UGCCU | | |
| hsa-miR- | GGGCGCCUGUGA | 30487 | GUUGGGAUCACAGG | 32529 | |
| 566 | UCCCAAC | | CGCCC | | |
| hsa-miR- | AGUAUGUUCUUC | 30488 | GUUCUGUCCUGGAA | 32530 | |
| 567 | CAGGACAGAAC | | GAACAUACU | | |
| hsa-miR- | GAGAAAUGCUGG | 30489 | GCAGAUUAGUCCAG | 32531 | |
| 5680 | ACUAAUCUGC | | CAUUUCUC | | |
| hsa-miR- | AGAAAGGGUGGC | 30490 | AAGAGGUAUUGCCA | 32532 | |
| 5681a | AAUACCUCUU | | CCCUUUCU | | |
| hsa-miR- | AGGUAUUGCCACC | 30491 | ACUAGAAAGGGUGG | 32533 | |
| 5681b | CUUUCUAGU | | CAAUACCU | | |
| hsa-miR- | GUAGCACCUUGCA | 30492 | ACCUUAUCCUGCAA | 32534 | |
| 5682 | GGAUAAGGU | | GGUGCUAC | | |
| hsa-miR- | UACAGAUGCAGA | 30493 | GAAGUCAGAGAAUC | 32535 | |
| 5683 | UUCUCUGACUUC | | UGCAUCUGUA | | |
| hsa-miR- | AACUCUAGCCUGA | 30494 | CUGUUGCUCAGGCU | 32536 | |
| 5684 | GCAACAG | | AGAGUU | | |
| hsa-miR- | ACAGCCCAGCAGU | 30495 | CCCGUGAUAACUGC | 32537 | |
| 5685 | UAUCACGGG | | UGGGCUGU | | |
| hsa-miR- | UAUCGUAUCGUA | 30496 | ACAAUACAAUACGA | 32538 | |
| 5686 | UUGUAUUGU | | UACGAUA | | |
| hsa-miR- | UUAGAACGUUUU | 30497 | AUUUGACCCUAAAA | 32539 | |
| $687 | AGGGUCAAAU | | CGUUCUAA | | |
| hsa-miR- | UAACAAACACCUG | 30498 | GCUGUUUUACAGGU | 32540 | |
| 5688 | UAAAACAGC | | GUUUGUUA | | |
| hsa-miR- | AGCAUACACCUGU | 30499 | UCUAGGACUACAGG | 32541 | |
| 5689 | AGUCCUAGA | | UGUAUGCU | | |
| hsa-miR- | AGUUAAUGAAUC | 30500 | ACUUUCCAGGAUUC | 32542 | |
| 569 | CUGGAAAGU | | AUUAACU | | |
| hsa-miR- | UCAGCUACUACCU | 30501 | CCUAAUAGAGGUAG | 32543 | |
| 5690 | CUAUUAGG | | UAGCUGA | | |
| hsa-miR- | UUGCUCUGAGCUC | 30502 | GCUUUCUCGGAGCU | 32544 | |
| 5691 | CGAGAAAGC | | CAGAGCAA | | |
| hsa-miR- | CAAAUAAUACCAC | 30503 | ACACCCACUGUGGU | 32545 | |
| 5692a | AGUGGGUGU | | AUUAUUUG | | |
| hsa-miR- | AAUAAUAUCACA | 30504 | ACACCUACUGUGAU | 32546 | |
| 5692b | GUAGGUGU | | AUUAUU | | |
| hsa-miR- | AAUAAUAUCACA | 30505 | GUACACCUACUGUG | 32547 | |
| 5692c | GUAGGUGUAC | | AUAUUAUU | | |
| hsa-miR- | GCAGUGGCUCUG | 30506 | GAGUUCAUUUCAGA | 32548 | |
| 5693 | AAAUGAACUC | | GCCACUGC | | |
| hsa-miR- | CAGAUCAUGGGA | 30507 | CUGAGACAGUCCCA | 32549 | |
| 5694 | CUGUCUCAG | | UGAUCUG | | |
| hsa-miR- | ACUCCAAGAAGA | 30508 | CUGUCUAGAUUCUU | 32550 | |
| 5695 | AUCUAGACAG | | CUUGGAGU | | |
| hsa-miR- | CUCAUUUAAGUA | 30509 | GGCAUCAGACUACU | 32551 | |
| 5696 | GUCUGAUGCC | | UAAAUGAG | | |
| hsa-miR- | UCAAGUAGUUUC | 30510 | CCUUUAUCAUGAAA | 32552 | |
| 5697 | AUGAUAAAGG | | CUACUUGA | | |
| hsa-miR- | UGGGGGAGUGCA | 30511 | CCACAAUCACUGCA | 32553 | |
| 5698 | GUGAUUGUGG | | CUCCCCCA | | |
| hsa-miR- | UCCUGUCUUUCCU | 30512 | GCUCCAACAAGGAA | 32554 | |
| 5699 | UGUUGGAGC | | AGACAGGA | | |
| hsa-miR- | UAAUGCAUUAAA | 30513 | CCUUCAAUAAUUUA | 32555 | |
| 5700 | UUAUUGAAGG | | AUGCAUUA | | |
| hsa-miR- | UUAUUGUCACGU | 30514 | AAUCAGAACGUGAC | 32556 | |
| 5701 | UCUGAUU | | AAUAA | | |
| hsa-miR- | UGAGUCAGCAAC | 30515 | CAUGGGAUAUGUUG | 32557 | |
| 5702 | AUAUCCCAUG | | CUGACUCA | | |
| hsa-miR- | AGGAGAAGUCGG | 30516 | ACCUUCCCGACUUC | 32558 | |
| 5703 | GAAGGU | | UCCU | | |
| hsa-miR- | CGAAAACAGCAA | 30517 | GCAAAGGUAAUUGC | 32559 | |
| 570-3p | UUACCUUUGC | | UGUUUUCG | | |
| hsa-miR- | UUAGGCCAUCAUC | 30518 | GCAUAAUGGGAUGA | 32560 | |
| 5704 | CCAUUAUGC | | UGGCCUAA | | |
| hsa-miR- | UGUUUCGGGGCU | 30519 | CACAGGCCAUGAGC | 32561 | |
| 5705 | CAUGGCCUGUG | | CCCGAAACA | | |
| hsa-miR- | AAAGGUAAUUGC | 30520 | GGGAAAAACUGCAA | 32562 | |
| 570-5p | AGUUUUUCCC | | UUACCUUU | | |
| hsa-miR- | UUCUGGAUAACA | 30521 | AGCUUCAGCAUGUU | 32563 | |
| 5706 | UGCUGAAGCU | | AUCCAGAA | | |
| hsa-miR- | ACGUUUGAAUGC | 30522 | GCCUUGUACAGCAU | 32564 | |
| 5707 | UGUACAAGGC | | UCAAACGU | | |
| hsa-miR- | AUGAGCGACUGU | 30523 | GGUCAGGCACAGUC | 32565 | |
| 5708 | GCCUGACC | | GCUCAU | | |
| hsa-miR- | GAGCCAGUUGGA | 30524 | GCUCCUGUCCAACU | 32566 | |
| 575 | CAGGAGC | | GGCUC | | |
| hsa-miR- | UUCAUUUGGUAU | 30525 | AAUCGCGGUUUAUA | 32567 | |
| 579 | AAACCGCGAUU | | CCAAAUGAA | | |
| hsa-miR- | UUGAGAAUGAUG | 30526 | CCUAAUGAUUCAUC | 32568 | |
| 580 | AAUCAUUAGG | | AUUCUCAA | | |
| hsa-miR- | UUACAGUUGUUC | 30527 | AGUAACUGGUUGAA | 32569 | |
| 582-5p | AACCAGUUACU | | CAACUGUAA | | |
| hsa-miR- | CAAAGAGGAAGG | 30528 | GUAAUGGGACCUUC | 32570 | |
| 583 | UCCCAUUAC | | CUCUUUG | | |
| hsa-miR- | UCAGUUCCAGGCC | 30529 | AGCCUGGUUGGCCU | 32571 | |
| 584-3p | AACCAGGCU | | GGAACUGA | | |
| hsa-miR- | UUAUGGUUUGCC | 30530 | CUCAGUCCCAGGCA | 32572 | |
| 584-5p | UGGGACUGAG | | AACCAUAA | | |
| hsa-miR- | UGGGCGUAUCUG | 30531 | UAGCAUACAGAUAC | 32573 | |
| 585 | UAUGCUA | | GCCCA | | |
| hsa-miR- | AGACCAUGGGUU | 30532 | ACAAUGAGAACCCA | 32574 | |
| 591 | CUCAUUGU | | UGGUCU | | |
| hsa-miR- | UUGUGUCAAUAU | 30533 | ACAUCAUCGCAUAU | 32575 | |
| 592 | GCGAUGAUGU | | UGACACAA | | |
| hsa-miR- | UGUCUCUGCUGG | 30534 | AGAAACCCCAGCAG | 32576 | |
| 593-3p | GGUUUCU | | AGACA | | |
| hsa-miR- | AGGCACCAGCCAG | 30535 | GCUGAGCAAUGCCU | 32577 | |
| 593-5p | GCAUUGCUCAGC | | GGCUGGUGCCU | | |
| hsa-miR- | GAAGUGUGCCGU | 30536 | AGACACACCACGGC | 32578 | |
| 595 | GGUGUGUCU | | ACACUUC | | |
| hsa-miR- | AAGCCUGCCCGGC | 30537 | CCCGAGGAGCCGGG | 32579 | |
| 596 | UCCUCGGG | | CAGGCUU | | |
| hsa-miR- | GUUGUGUCAGUU | 30538 | GUUUGAUAAACUGA | 32580 | |
| 599 | UAUCAAAC | | CACAAC | | |
| hsa-miR- | UGGUCUAGGAUU | 30539 | CUCCUCCAACAAUC | 32581 | |
| 601 | GUUGGAGGAG | | CUAGACCA | | |
| hsa-miR- | CACACACUGCAAU | 30540 | GCAAAAGUAAUUGC | 32582 | |
| 603 | UACUUUUGC | | AGUGUGUG | | |
| hsa-miR- | AGGGGUGGUGUU | 30541 | ACGGAGCUGUCCCA | 32583 | |
| 608 | GGGACAGCUCCGU | | ACACCACCCCU | | |
| hsa-miR- | UGAGCUAAAUGU | 30542 | UCCCAGCACACAUU | 32584 | |
| 610 | GUGCUGGGA | | UAGCUCA | | |
| hsa-miR- | GCGAGGACCCCUC | 30543 | GUCAGACCCCGAGG | 32585 | |
| 611 | GGGGUCUGAC | | GGUCCUCGC | | |
| hsa-miR- | GCUGGGCAGGGC | 30544 | AAGGAGCUCAGAAG | 32586 | |
| 612 | UUCUGAGCUCCUU | | CCCUGCCCAGC | | |
| hsa-miR- | GGGAAAAGGAAG | 30545 | UCCUCCCCCUUCCU | 32587 | |
| 6124 | GGGGAGGA | | UUUCCC | | |
| hsa-miR- | GCGGAAGGCGGA | 30546 | UCCGCCGCUCCGCC | 32588 | |
| $125 | GCGGCGGA | | UUCCGC | | |
| hsa-miR- | GUGAAGGCCCGGC | 30547 | UCUCCGCCGGGCCU | 32589 | |
| 6126 | GGAGA | | UCAC | | |
| hsa-miR- | UGAGGGAGUGGG | 30548 | CCUCCCACCCACUC | 32590 | |
| 6127 | UGGGAGG | | CCUCA | | |
| hsa-miR- | ACUGGAAUUGGA | 30549 | UUUUGACUCCAAUU | 32591 | |
| 5128 | GUCAAAA | | CCAGU | | |
| hsa-miR- | UGAGGGAGUUGG | 30550 | UAUACACCCAACUC | 32592 | |
| 6129 | GUGUAUA | | CCUCA | | |
| hsa-miR- | UGAGGGAGUGGA | 30551 | CAUACAAUCCACUC | 32593 | |
| 6130 | UUGUAUG | | CCUCA | | |
| hsa-miR- | GGCUGGUCAGAU | 30552 | CACUCCCAUCUGAC | 32594 | |
| 6131 | GGGAGUG | | CAGCC | | |
| hsa-miR- | AGCAGGGCUGGG | 30553 | UGCAAUCCCCAGCC | 32595 | |
| 6132 | GAUUGCA | | CUGCU | | |
| hsa-miR- | UGAGGGAGGAGG | 30554 | UACCCAACCUCCUC | 32596 | |
| 6133 | UUGGGUA | | CCUCA | | |
| hsa-miR- | UGAGGUGGUAGG | 30555 | UCUACAUCCUACCA | 32597 | |
| 6134 | AUGUAGA | | CCUCA | | |
| hsa-miR- | UCCGAGCCUGGGU | 30556 | AAGAGGGAGACCCA | 32598 | |
| 615-3p | CUCCCUCUU | | GGCUCGGA | | |
| hsa-miR- | GGGGGUCCCCGGU | 30557 | GAUCCGAGCACCGG | 32599 | |
| 615-5p | GCUCGGAUC | | GGACCCCC | | |
| hsa-miR- | AGUCAUUGGAGG | 30558 | CUGCUCAAACCCUC | 32600 | |
| 616-3p | GUUUGAGCAG | | CAAUGACU | | |
| hsa-miR- | CAGCAGGAGGUG | 30559 | CUCCCCUCACCUCC | 32601 | |
| 6165 | AGGGGAG | | UGCUG | | |
| hsa-miR- | ACUCAAAACCCUU | 30560 | AAGUCACUGAAGGG | 32602 | |
| 616-5p | CAGUGACUU | | UUUUGAGU | | |
| hsa-miR- | AGACUUCCCAUUU | 30561 | GCCACCUUCAAAUG | 32603 | |
| 617 | GAAGGUGGC | | GGAAGUCU | | |
| hsa-miR- | AAACUCUACUUG | 30562 | ACUCAGAAGGACAA | 32604 | |
| 618 | UCCUUCUGAGU | | GUAGAGUUU | | |
| hsa-miR- | GGCUAGCAACAGC | 30563 | AGGUAAGCGCUGUU | 32605 | |
| 621 | GCUUACCU | | GCUAGCC | | |
| hsa-miR- | ACAGUCUGCUGA | 30564 | GCUCCAACCUCAGC | 32606 | |
| 622 | GGUUGGAGC | | AGACUGU | | |
| hsa-miR- | AUCCCUUGCAGGG | 30565 | ACCCAACAGCCCCU | 32607 | |
| 623 | GCUGUUGGGU | | GCAAGGGAU | | |
| hsa-miR- | GUGAGUCUCUAA | 30566 | UCCUCUUUUCUUAG | 32608 | |
| 627 | GAAAAGAGGA | | AGACUCAC | | |
| hsa-miR- | UCUAGUAAGAGU | 30567 | UCGACUGCCACUCU | 32609 | |
| 628-3p | GGCAGUCGA | | UACUAGA | | |
| hsa-miR- | AUGCUGACAUAU | 30568 | CCUCUAGUAAAUAU | 32610 | |
| 628-5p | UUACUAGAGG | | GUCAGCAU | | |
| hsa-miR- | GUUCUCCCAACGU | 30569 | GCUGGGCUUACGUU | 32611 | |
| 629-3p | AAGCCCAGC | | GGGAGAAC | | |
| hsa-miR- | UGGGUUUACGUU | 30570 | AGUUCUCCCAACGU | 32612 | |
| 629-5p | GGGAGAACU | | AAACCCA | | |
| hsa-miR- | GUGUCUGCUUCCU | 30571 | UCCCACAGGAAGCA | 32613 | |
| 632 | GUGGGA | | GACAC | | |
| hsa-miR- | CUAAUAGUAUCU | 30572 | UUUAUUGUGGUAGA | 32614 | |
| 633 | ACCACAAUAAA | | UACUAUUAG | | |
| hsa-miR- | UGUGCUUGCUCG | 30573 | UGCGGGCGGGACGA | 32615 | |
| 636 | UCCCGCCCGCA | | GCAAGCACA | | |
| hsa-miR- | AGGGAUCGCGGG | 30574 | AGGCCGCCACCCGC | 32616 | |
| 638 | CGGGUGGCGGCCU | | CCGCGAUCCCU | | |
| hsa-miR- | AUGAUCCAGGAA | 30575 | AGAGGCAGGUUCCU | 32617 | |
| 640 | CCUGCCUCU | | GGAUCAU | | |
| hsa-miR- | AGUGUGGCUUUC | 30576 | GCUCUAAGAAAGCC | 32618 | |
| 644a | UUAGAGC | | ACACU | | |
| hsa-miR- | UCUAGGCUGGUA | 30577 | UCAGCAGUACCAGC | 32619 | |
| 645 | CUGCUGA | | CUAGA | | |
| hsa-miR- | AAGCAGCUGCCUC | 30578 | GCCUCAGAGGCAGC | 32620 | |
| 646 | UGAGGC | | UGCUU | | |
| hsa-miR- | GUGGCUGCACUCA | 30579 | GAAGGAAGUGAGUG | 32621 | |
| 647 | CUUCCUUC | | CAGCCAC | | |
| hsa-miR- | AGGAGGCAGCGC | 30580 | GUCCUGAGAGCGCU | 32622 | |
| 650 | UCUCAGGAC | | GCCUCCU | | |
| hsa-miR- | AAUGGCGCCACUA | 30581 | CACAACCCUAGUGG | 32623 | |
| 652-3p | GGGUUGUG | | CGCCAUU | | |
| hsa-miR- | CAACCCUAGGAGA | 30582 | UGAAUGGCACCCUC | 32624 | |
| 652-5p | GGGUGCCAUUCA | | UCCUAGGGUUG | | |
| hsa-miR- | AUAAUACAUGGU | 30583 | AAAGAGGUUAACCA | 32625 | |
| 655 | UAACCUCUUU | | UGUAUUAU | | |
| hsa-miR- | AAUAUUAUACAG | 30584 | AGAGGUUGACUGUA | 32626 | |
| 656 | UCAACCUCU | | UAAUAUU | | |
| hsa-miR- | GGCGGAGGGAAG | 30585 | ACCAACGGACCUAC | 32627 | |
| 658 | UAGGUCCGUUGG | | UUCCCUCCGCC | | |
| | U | | | | |
| hsa-miR- | UGCCUGGGUCUCU | 30586 | ACGCGCAGGCCAGA | 32628 | |
| 661 | GGCCUGCGCGU | | GACCCAGGCA | | |
| hsa-miR- | AGGCGGGGCGCCG | 30587 | GCGGUCCCGCGGCG | 32629 | |
| 663a | CGGGACCGC | | CCCCGCCU | | |
| hsa-miR- | GGUGGCCCGGCCG | 30588 | CCUCAGGCACGGCC | 32630 | |
| 663b | UGCCUGAGG | | GGGCCACC | | |
| hsa-miR- | ACCAGGAGGCUG | 30589 | AGGGGCCUCAGCCU | 32631 | |
| 665 | AGGCCCCU | | CCUGGU | | |
| hsa-miR- | GUCCCUGAGUGU | 30590 | CACCACAUACACUC | 32632 | |
| 670 | AUGUGGUG | | AGGGAC | | |
| hsa-miR- | UCCGGUUCUCAGG | 30591 | GGUGGAGCCCUGAG | 32633 | |
| 671-3p | GCUCCACC | | AACCGGA | | |
| hsa-miR- | AGGAAGCCCUGG | 30592 | CUCCAGCCCCUCCA | 32634 | |
| 671-5p | AGGGGCUGGAG | | GGGCUUCCU | | |
| hsa-miR- | CUGUAUGCCCUCA | 30593 | UGAGCGGUGAGGGC | 32635 | |
| 675-3p | CCGCUCA | | AUACAG | | |
| hsa-miR- | UGGUGCGGAGAG | 30594 | CACUGUGGGCCCUC | 32636 | |
| 675-5p | GGCCCACAGUG | | UCCGCACCA | | |
| hsa-miR- | CAACUAGACUGU | 30595 | CUAGAAGCUCACAG | 32637 | |
| 708-3p | GAGCUUCUAG | | UCUAGUUG | | |
| hsa-miR- | AAGGAGCUUACA | 30596 | CCCAGCUAGAUUGU | 32638 | |
| 708-5p | AUCUAGCUGGG | | AAGCUCCUU | | |
| hsa-miR- | GGGACCCAGGGA | 30597 | CUUACGUCUCUCCC | 32639 | |
| 711 | GAGACGUAAG | | UGGGUCCC | | |
| hsa-miR- | GCAGAGUGCAAA | 30598 | GUCAAAAUUGUUUG | 32640 | |
| 759 | CAAUUUUGAC | | CACUCUGC | | |
| hsa-miR- | CGGCUCUGGGUCU | 30599 | UCCCCACAGACCCA | 32641 | |
| 760 | GUGGGGA | | GAGCCG | | |
| hsa-miR- | GCAGCAGGGUGA | 30600 | UGUGUCAGUUUCAC | 32642 | |
| 761 | AACUGACACA | | CCUGCUGC | | |
| hsa-miR- | UGGAGGAGAAGG | 30601 | CAUCACCUUCCUUC | 32643 | |
| 765 | AAGGUGAUG | | UCCUCCA | | |
| hsa-miR- | UCUGCUCAUACCC | 30602 | AGAAACCAUGGGGU | 32644 | |
| 767-3p | CAUGGUUUCU | | AUGAGCAGA | | |
| hsa-miR- | UGCACCAUGGUU | 30603 | CAUGCUCAGACAAC | 32645 | |
| 767-5p | GUCUGAGCAUG | | CAUGGUGCA | | |
| hsa-miR- | CUGGGAUCUCCGG | 30604 | AACCAAGACCCCGG | 32646 | |
| 769-3p | GGUCUUGGUU | | AGAUCCCAG | | |
| hsa-miR- | UGAGACCUCUGG | 30605 | AGCUCAGAACCCAG | 32647 | |
| 769-5p | GUUCUGAGCU | | AGGUCUCA | | |
| hsa-miR- | UCCAGUACCACGU | 30606 | UGGCCCUGACACGU | 32648 | |
| 770-5p | GUCAGGGCCA | | GGUACUGGA | | |
| hsa-miR- | GGAGACUGAUGA | 30607 | UCCCGGGAACUCAU | 32649 | |
| 873-3p | GUUCCCGGGA | | CAGUCUCC | | |
| hsa-miR- | GCAGGAACUUGU | 30608 | AGGAGACUCACAAG | 32650 | |
| 873-5p | GAGUCUCCU | | UUCCUGC | | |
| hsa-miR- | CUGCCCUGGCCCG | 30609 | UCGGUCCCUCGGGC | 32651 | |
| 874 | AGGGACCGA | | CAGGGCAG | | |
| hsa-miR- | CCUGGAAACACUG | 30610 | CACAACCUCAGUGU | 32652 | |
| 875-3p | AGGUUGUG | | UUCCAGG | | |
| hsa-miR- | UAUACCUCAGUU | 30611 | CACCUGAUAAAACU | 32653 | |
| 875-5p | UUAUCAGGUG | | GAGGUAUA | | |
| hsa-miR- | UGGUGGUUUACA | 30612 | UGAAUUACUUUGUA | 32654 | |
| 876-3p | AAGUAAUUCA | | AACCACCA | | |
| hsa-miR- | UGGAUUUCUUUG | 30613 | UGGUGAUUCACAAA | 32655 | |
| 876-5p | UGAAUCACCA | | GAAAUCCA | | |
| hsa-miR- | UCCUCUUCUCCCU | 30614 | CUGGGAGGAGGGAG | 32656 | |
| 877-3p | CCUCCCAG | | AAGAGGA | | |
| hsa-miR- | GUAGAGGAGAUG | 30615 | CCCUGCGCCAUCUC | 32657 | |
| 877-5p | GCGCAGGG | | CUCUAC | | |
| hsa-miR- | GUGAACGGGCGCC | 30616 | CCUCGGGAUGGCGC | 32658 | |
| 887 | AUCCCGAGG | | CCGUUCAC | | |
| hsa-miR- | GACUGACACCUCU | 30617 | UUCACCCAAAGAGG | 32659 | |
| 888-3p | UUGGGUGAA | | UGUCAGUC | | |
| hsa-miR- | UACUCAAAAAGC | 30618 | UGACUGACAGCUUU | 32660 | |
| 888-5p | UGUCAGUCA | | UUGAGUA | | |
| hsa-miR- | UUAAUAUCGGAC | 30619 | ACAAUGGUUGUCCG | 32661 | |
| 889 | AACCAUUGU | | AUAUUAA | | |
| hsa-miR- | AUCCGOGCUCUGA | 30620 | GGCAGAGAGUCAGA | 32662 | |
| 937-3p | CUCUCUGCC | | GCGCGGAU | | |
| hsa-miR- | GUGAGUCAGGGU | 30621 | CCAGCCCCACCCUG | 32663 | |
| 937-5p | GGGGCUGG | | ACUCAC | | |
| hsa-miR- | UGCCCUUAAAGG | 30622 | ACUGGGUUCACCUU | 32664 | |
| 938 | UGAACCCAGU | | UAAGGGCA | | |
| hsa-miR- | UCUUCUCUGUUU | 30623 | CACAUGGCCAAAAC | 32665 | |
| 942 | UGGCCAUGUG | | AGAGAAGA | | |
| hsa-miR- | AAAUUAUUGUAC | 30624 | CUCAUCCGAUGUAC | 32666 | |
| 944 | AUCGGAUGAG | | AAUAAUUU | | |
| hsa-miR-95 | UUCAACGGGUAU | 30625 | UGCUCAAUAAAUAC | 32667 | |
| | UUAUUGAGCA | | CCGUUGAA | | |
| hsa-miR- | CUAUACAACUUAC | 30626 | GGGAAAGUAGUAAG | 32668 | |
| 98-3p | UACUUUCCC | | UUGUAUAG | | |
| hsa-miR- | UGAGGUAGUAAG | 30627 | AACAAUACAACUUA | 32669 | |
| 98-5p | UUGUAUUGUU | | CUACCUCA | | |

### II. Formulation and Delivery

### Pharmaceutical Compositions

According to the present disclosure the viral particles may be prepared as pharmaceutical compositions. It will be understood that such compositions necessarily comprise one or more active ingredients and, most often, a pharmaceutically acceptable excipient.

Relative amounts of the active ingredient (e.g. viral particle), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the viral particle pharmaceutical compositions described herein may comprise at least one payload. As a non-limiting example, the pharmaceutical compositions may contain a viral particle with 1, 2, 3, 4 or 5 payloads. In some embodiments, the pharmaceutical composition may contain a nucleic acid encoding a payload construct encoding proteins selected from antibodies and/or antibody-based compositions.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, rats, birds, including commercially relevant birds such as poultry, chickens, ducks, geese, and/or turkeys.

In some embodiments, compositions are administered to humans, human patients, or subjects.

### Formulations

The viral particles of the disclosure can be formulated using one or more excipients to: (1) increase stability; (2) increase cell transfection or transduction; (3) permit the sustained or delayed expression of the payload; (4) alter the biodistribution (e.g., target the viral particle to specific tissues or cell types); (5) increase the translation of encoded protein; (6) alter the release profile of encoded protein; and/or (7) allow for regulatable expression of the payload.

Formulations of the present disclosure can include, without limitation, saline, liposomes, lipid nanoparticles, polymers, peptides, proteins, cells transfected with viral vectors (e.g., for transfer or transplantation into a subject) and combinations thereof.

Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods include the step of associating the active ingredient with an excipient and/or one or more other accessory ingredients. As used herein, the phrase “active ingredient” generally refers either to a viral particle carrying a payload region encoding the polypeptides of the disclosure or to the antibody or antibody-based composition encoded by a viral genome of by a viral particle as described herein.

Formulations of the viral particles and pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In some embodiments, the viral particles of the disclosure may be formulated in PBS with 0.001% of Pluronic acid (F-68) at a pH of about 7.0.

Relative amounts of the active ingredient (e.g. viral particle), the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, or at least 80% (w/w) active ingredient.

In some embodiments, the AAV formulations described herein may contain sufficient viral particles for expression of at least one expressed functional antibody or antibody-based composition. As a non-limiting example, the viral particles may contain viral genomes encoding 1, 2, 3, 4, or 5 functional antibodies.

According to the present disclosure viral particles may be formulated for CNS delivery. Agents that cross the brain blood barrier may be used. For example, some cell penetrating peptides that can target molecules to the brain blood barrier endothelium may be used for formulation (e.g., Mathupala, Expert Opin Ther Pat., 2009, 19, 137-140: the content of which is incorporated herein by reference in its entirety).

### Excipients and Diluents

The viral particles of the disclosure can be formulated using one or more excipients or diluents to (1) increase stability; (2) increase cell transfection or transduction; (3) permit the sustained or delayed release; (4) alter the biodistribution (e.g., target the viral particle to specific tissues or cell types); (5) increase the translation of encoded protein in vivo; (6) alter the release profile of encoded protein in vivo; and; or (7) allow for regulatable expression of the polypeptides of the disclosure.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, M D, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

### Inactive Ingredients

In some embodiments, viral particle formulations may comprise at least one inactive ingredient. As used herein, the term “inactive ingredient” refers to one or more agents that do not contribute to the activity of the active ingredient of the pharmaceutical composition included in formulations. In some embodiments, all, none or some of the inactive ingredients which may be used in the formulations of the present disclosure may be approved by the US Food and Drug Administration (FDA).

In some embodiments, the viral particle pharmaceutical compositions comprise at least one inactive ingredient such as, but not limited to, 1,2,6-Hexanetriol; 1,2-Dimyristoyl-Sn-Glycero-3-(Phospho-S-(1-Glycerol)); 1,2-Dimyristoyl-Sn-Glycero-3-Phosphocholine; 1,2-Dioleoyl-Sn-Glycero-3-Phosphocholine; 1,2-Dipalmitoyl-Sn-Glycero-3-(Phospho-Rac-(1-Glycerol)); 1,2-Distearoyl-Sn-Glycero-3-(Phospho-Rac-(1-Glycerol)); 1,2-Distearoyl-Sn-Glycero-3-Phosphocholine; 1-O-Tolylbiguanide; 2-Ethyl-1,6-Hexanediol; Acetic Acid; Acetic Acid, Glacial; Acetic Anhydride; Acetone; Acetone Sodium Bisulfate; Acetylated Lanolin Alcohols; Acetylated Monoglycerides; Acetylcysteine; Acetyltryptophan, DL-; Acrylates Copolymer; Acrylic Acid-Isooctyl Acrylate Copolymer; Acrylic Adhesive 788; Activated Charcoal; Adcote 72A103; Adhesive Tape; Adipic Acid; Aerotex Resin 3730; Alanine; Albumin Aggregated; Albumin Colloidal; Albumin Human; Alcohol; Alcohol, Dehydrated; Alcohol, Denatured; Alcohol, Diluted; Alfadex; Alginic Acid; Alkyl Ammonium Sulfonic Acid Betaine; Alkyl Aryl Sodium Sulfonate; Allantoin; Allyl .Alpha.-Ionone; Almond Oil; Alpha-Terpineol; Alpha-Tocopherol; Alpha-Tocopherol Acetate, D1-; Alpha-Tocopherol, D1-; Aluminum Acetate; Aluminum Chlorhydroxy Allantoinate; Aluminum Hydroxide; Aluminum Hydroxide-Sucrose, Hydrated; Aluminum Hydroxide Gel; Aluminum Hydroxide Gel F 500; Aluminum Hydroxide Gel F 5000; Aluminum Monostearate; Aluminum Oxide; Aluminum Polyester; Aluminum Silicate; Aluminum Starch Octenylsuccinate; Aluminum Stearate; Aluminum Subacetate; Aluminum Sulfate Anhydrous; Amerchol C; Amerchol-Cab; Aminomethylpropanol; Ammonia; Ammonia Solution; Ammonia Solution, Strong; Ammonium Acetate; Ammonium Hydroxide; Ammonium Lauryl Sulfate; Ammonium Nonoxynol-4 Sulfate; Ammonium Salt Of C-12-C-15 Linear Primary Alcohol Ethoxylate; Ammonium Sulfate; Ammonyx; Amphoteric-2; Amphoteric-9; Anethole; Anhydrous Citric Acid; Anhydrous Dextrose; Anhydrous Lactose; Anhydrous Trisodium Citrate; Aniseed Oil; Anoxid Sbn; Antifoam; Antipyrine; Apaflurane; Apricot Kernel Oil Peg-6 Esters; Aquaphor; Arginine; Arlacel; Ascorbic Acid; Ascorbyl Palmitate; Aspartic Acid; Balsam Peru; Barium Sulfate; Beeswax; Beeswax, Synthetic; Beheneth-10; Bentonite; Benzalkonium Chloride; Benzenesulfonic Acid; Benzethonium Chloride; Benzododecinium Bromide; Benzoic Acid; Benzyl Alcohol; Benzyl Benzoate; Benzyl Chloride; Betadex; Bibapcitide; Bismuth Subgallate; Boric Acid; Brocrinat; Butane; Butyl Alcohol; Butyl Ester Of Vinyl Methyl Ether/Maleic Anhydride Copolymer (125000 Mw); Butyl Stearate; Butylated Hydroxyanisole; Butylated Hydroxytoluene; Butylene Glycol; Butylparaben; Butyric Acid; C20-40 Paneth-24; Caffeine; Calcium; Calcium Carbonate; Calcium Chloride; Calcium Gluceptate; Calcium Hydroxide; Calcium Lactate; Calcobutrol; Caldiamide Sodium; Caloxetate Trisodium; Calteridol Calcium; Canada Balsam; Caprylic/Capric Triglyceride; Caprylic/Capric/Stearic Triglyceride; Captan; Captisol; Caramel; Carbomer 1342; Carbomer 1382; Carbomer 934; Carbomer 934p; Carbomer 940; Carbomer 941; Carbomer 980; Carbomer 981; Carbomer Homopolymer Type B (Allyl Pentaerythritol Crosslinked); Carbomer Homopolymer Type C (Allyl Pentaerythritol Crosslinked); Carbon Dioxide; Carboxy Vinyl Copolymer; Carboxymethylcellulose; Carboxymethylcellulose Sodium; Carboxypolymethylene; Carrageenan; Carrageenan Salt; Castor Oil; Cedar Leaf Oil; Cellulose; Cellulose, Microcrystalline; Cerasynt-Se; Ceresin; Ceteareth-12; Ceteareth-15; Ceteareth-30; Cetearyl Alcohol/Ceteareth-20; Cetearyl Ethylhexanoate; Ceteth-10; Ceteth-2; Ceteth-20; Ceteth-23; Cetostearyl Alcohol; Cetrimonium Chloride; Cetyl Alcohol; Cetyl Esters Wax; Cetyl Palmitate; Cetylpyridinium Chloride; Chlorobutanol; Chlorobutanol Hemihydrate; Chlorobutanol, Anhydrous; Chlorocresol; Chloroxylenol; Cholesterol; Choleth; Choleth-24; Citrate; Citric Acid; Citric Acid Monohydrate; Citric Acid, Hydrous; Cocamide Ether Sulfate; Cocamine Oxide; Coco Betaine; Coco Diethanolamide; Coco Monoethanolamide; Cocoa Butter; Coco-Glycerides; Coconut Oil; Coconut Oil, Hydrogenated; Coconut Oil/Palm Kernel Oil Glycerides, Hydrogenated; Cocoyl Caprylocaprate; Cola Nitida Seed Extract; Collagen; Coloring Suspension; Corn Oil; Cottonseed Oil; Cream Base; Creatine; Creatinine; Cresol; Croscarmellose Sodium; Crospovidone; Cupric Sulfate; Cupric Sulfate Anhydrous; Cyclomethicone; Cyclomethicone/Dimethicone Copolyol; Cysteine; Cysteine Hydrochloride; Cysteine Hydrochloride Anhydrous; Cysteine, D1-; D&C Red No. 28; D&C Red No. 33; D&C Red No. 36; D&C Red No. 39; D&C Yellow No. 10; Dalfampridine; Daubert 1-5 Pestr (Matte) 164z; Decyl Methyl Sulfoxide; Dehydag Wax Sx; Dehydroacetic Acid; Dehymuls E; Denatonium Benzoate; Deoxycholic Acid; Dextran; Dextran 40; Dextrin; Dextrose; Dextrose Monohydrate; Dextrose Solution; Diatrizoic Acid; Diazolidinyl Urea; Dichlorobenzyl Alcohol; Dichlorodifluoromethane; Dichlorotetrafluoroethane; Diethanolamine; Diethyl Pyrocarbonate; Diethyl Sebacate; Diethylene Glycol Monoethyl Ether; Diethylhexyl Phthalate; Dihydroxyaluminum Aminoacetate; Diisopropanolamine; Diisopropyl Adipate; Diisopropyl Dilinoleate; Dimethicone 350; Dimethicone Copolyol; Dimethicone Mdx4-4210; Dimethicone Medical Fluid 360; Dimethyl Isosorbide; Dimethyl Sulfoxide; Dimethylaminoethyl Methacrylate-Butyl Methacrylate-Methyl Methacrylate Copolymer; Dimethyldioctadecylammonium Bentonite; Dimethylsiloxane/Methylvinylsiloxane Copolymer; Dinoseb Ammonium Salt; Dipalmitoylphosphatidylglycerol, D1-; Dipropylene Glycol; Disodium Cocoamphodiacetate; Disodium Laureth Sulfosuccinate; Disodium Lauryl Sulfosuccinate; Disodium Sulfosalicylate; Disofenin; Divinylbenzene Styrene Copolymer; Dmdm Hydantoin; Docosanol; Docusate Sodium; Duro-Tak 280-2516; Duro-Tak 387-2516; Duro-Tak 80-1196; Duro-Tak 87-2070; Duro-Tak 87-2194; Duro-Tak 87-2287; Duro-Tak 87-2296; Duro-Tak 87-2888; Duro-Tak 87-2979; Edetate Calcium Disodium; Edetate Disodium; Edetate Disodium Anhydrous; Edetate Sodium; Edetic Acid; Egg Phospholipids; Entsufon; Entsufon Sodium; Epilactose; Epitetracycline Hydrochloride; Essence Bouquet 9200; Ethanolamine Hydrochloride; Ethyl Acetate; Ethyl Oleate; Ethylcelluloses; Ethylene Glycol; Ethylene Vinyl Acetate Copolymer; Ethylenediamine; Ethylenediamine Dihydrochloride; Ethylene-Propylene Copolymer; Ethylene-Vinyl Acetate Copolymer (28% Vinyl Acetate); Ethylene-Vinyl Acetate Copolymer (9% Vinylacetate); Ethylhexyl Hydroxystearate; Ethylparaben; Eucalyptol; Exametazime; Fat, Edible; Fat, Hard; Fatty Acid Esters; Fatty Acid Pentaerythriol Ester; Fatty Acids; Fatty Alcohol Citrate; Fatty Alcohols; Fd&C Blue No. I; Fd&C Green No. 3; Fd&C Red No. 4; Fd&C Red No. 40; Fd&C Yellow No. 10 (Delisted); Fd&C Yellow No. 5; Fd&C Yellow No. 6; Ferric Chloride; Ferric Oxide; Flavor 89-186; Flavor 89-259; Flavor Df-119; Flavor Df-1530; Flavor Enhancer; Flavor Fig 827118; Flavor Raspberry Pfc-8407; Flavor Rhodia Pharmaceutical No. Rf 451; Fluorochlorohydrocarbons; Formaldehyde; Formaldehyde Solution; Fractionated Coconut Oil; Fragrance 3949-5; Fragrance 520a; Fragrance 6.007; Fragrance 91-122; Fragrance 9128-Y; Fragrance 93498g; Fragrance Balsam Pine No, 5124; Fragrance Bouquet 10328; Fragrance Chemoderm 6401-B; Fragrance Chemoderm 6411; Fragrance Cream No. 73457; Fragrance Cs-28197; Fragrance Felton 066m; Fragrance Firmenich 47373; Fragrance Givaudan Ess 9090/1c; Fragrance 11-6540; Fragrance Herbal 10396; Fragrance Nj-1085; Fragrance P O F1-147; Fragrance Pa 52805; Fragrance Pera Derm D; Fragrance Rbd-9819; Fragrance Shaw Mudge U-7776; Fragrance Tf 044078; Fragrance Lingerer Honeysuckle K 2771; Fragrance lingerer N5195; Fructose; Gadolinium Oxide; Galactose; Gamma Cyclodextrin; Gelatin; Gelatin, Crosslinked; Gelfoam Sponge; Gellan Gum (Low Acyl); Gelva 737; Gentisic Acid; Gentisic Acid Ethanolamide; Gluceptate Sodium; Gluceptate Sodium Dihydrate; Gluconolactone; Glucuronic Acid; Glutamic Acid, D1-; Glutathione; Glycerin; Glycerol Ester Of Hydrogenated Rosin; Glyceryl Citrate; Glyceryl Isostearate; Glyceryl Laurate; Glyceryl Monostearate; Glyceryl Oleate; Glyceryl Oleate/Propylene Glycol; Glyceryl Palmitate; Glyceryl Ricinoleate; Glyceryl Stearate; Glyceryl Stearate Laureth-23; Glyceryl Stearate/Peg Stearate; Glyceryl Stearate/Peg-100 Stearate; Glyceryl Stearate/Peg-40 Stearate; Glyceryl Stearate-Stearamidoethyl Diethylamine; Glyceryl Trioleate; Glycine; Glycine Hydrochloride; Glycol Distearate; Glycol Stearate; Guanidine Hydrochloride; Guar Gum; Hair Conditioner (18n195-1m); Heptane; Hetastarch; Hexylene Glycol; High Density Polyethylene; Histidine; Human Albumin Microspheres; Hyaluronate Sodium; Hydrocarbon; Hydrocarbon Gel, Plasticized; Hydrochloric Acid; Hydrochloric Acid, Diluted; Hydrocortisone; Hydrogel Polymer; Hydrogen Peroxide; Hydrogenated Castor Oil; Hydrogenated Palm Oil; Hydrogenated Palm/Palm Kernel Oil Peg-6 Esters; Hydrogenated Polybutene 635-690; Hydroxide Ion; Hydroxyethyl Cellulose; Hydroxyethylpiperazine Ethane Sulfonic Acid; Hydroxymethyl Cellulose; Hydroxyoctacosanyl Hydroxystearate; Hydroxypropyl Cellulose; Hydroxypropyl Methylcellulose 2906; Hydroxypropyl-Beta-cyclodextrin; Hypromellose 2208 (15000 Mpa·S); Hypromellose 2910 (15000 Mpa·S); Hypromelloses; Imidurea; Iodine; Iodoxamic Acid; Iofetamine Hydrochloride; Irish Moss Extract; Isobutane; Isoceteth-20; Isoleucine; Isooctyl Acrylate; Isopropyl Alcohol; Isopropyl Isostearate; Isopropyl Myristate; Isopropyl Myristate m Myristyl Alcohol; Isopropyl Palmitate; Isopropyl Stearate; Isostearic Acid; Isostearyl Alcohol; Isotonic Sodium Chloride Solution; Jelene; Kaolin; Kathon Cg; Kathon Cg E; Lactate; Lactic Acid; Lactic Acid, D1-; Lactic Acid, L-; Lactobionic Acid; Lactose; Lactose Monohydrate; Lactose, Hydrous; Laneth; Lanolin; Lanolin Alcohol-Mineral Oil; Lanolin Alcohols; Lanolin Anhydrous; Lanolin Cholesterols; Lanolin Nonionic Derivatives; Lanolin, Ethoxylated; Lanolin, Hydrogenated; Lauralkonium Chloride; Lauramine Oxide; Laurdimonium Hydrolyzed Animal Collagen; Laureth Sulfate; Laureth-2; Laureth-23; Laureth-4; Laurie Diethanolamide; Laurie Myristic Diethanolamide; Lauroyl Sarcosine; Lauryl Lactate; Lauryl Sulfate; Lavandula Angustifolia Flowering Top; Lecithin; Lecithin Unbleached; Lecithin, Egg; Lecithin, Hydrogenated; Lecithin, Hydrogenated Soy; Lecithin, Soybean; Lemon Oil; Leucine; Levulinic Acid; Lidofenin; Light Mineral Oil; Light Mineral Oil (85 Ssu); Limonene, (+/−)-; Lipocol Se-15; Lysine; Lysine Acetate; Lysine Monohydrate; Magnesium Aluminum Silicate; Magnesium Aluminum Silicate Hydrate; Magnesium Chloride; Magnesium Nitrate; Magnesium Stearate; Maleic Acid; Mannitol; Maprofix; Mebrofenin; Medical Adhesive Modified S-15; Medical Antiform A-F Emulsion; Medronate Disodium; Medronic Acid; Meglumine; Menthol; Metacresol; Metaphosphoric Acid; Methanesulfonic Acid; Methionine; Methyl Alcohol; Methyl Gluceth-10; Methyl Gluceth-20; Methyl Gluceth-20 Sesquistearate; Methyl Glucose Sesquistearate; Methyl Laurate; Methyl Pyrrolidone; Methyl Salicylate; Methyl Stearate; Methylboronic Acid; Methylcellulose (4000 Mpa·S); Methylcelluloses; Methylchloroisothiazolinone; Methylene Blue; Methylisothiazolinone; Methylparaben; Microcrystal line Wax; Mineral Oil; Mono and Diglyceride; Monostearyl Citrate; Monothioglycerol; Multisterol Extract; Myristyl Alcohol; Myristyl Lactate; Myristyl-.Gamma.-Picolinium Chloride; N-(Carbamoyl-Methoxy Peg-40)-1,2-Distearoyl-Cephalin Sodium; N,N-Dimethylacetamide; Niacinamide; Nioxime; Nitric Acid; Nitrogen; Nonoxynol Iodine; Nonoxynol-15; Nonoxynol-9; Norflurane; Oatmeal; Octadecene-1/Maleic Acid Copolymer; Octanoic Acid; Octisalate; Octoxynol-1; Octoxynol-40; Octoxynol-9; Octyldodecanol; Octylphenol Polymethylene; Oleic Acid; Oleth-10/01eth-5; Oleth-2; Oleth-20; Oleyl Alcohol; Oleyl Oleate; Olive Oil; Oxidronate Disodium; Oxyquinoline; Palm Kernel Oil; Palmitamine Oxide; Parabens; Paraffin; Paraffin, White Soft; Parfum Creme 45/3; Peanut Oil; Peanut Oil, Refined; Pectin; Peg 6-32 Stearate/Glycol Stearate; Peg Vegetable Oil; Peg-100 Stearate; Peg-12 Glyceryl Laurate; Peg-120 Glyceryl Stearate; Peg-120 Methyl Glucose Dioleate; Peg-15 Cocamine; Peg-150 Distearate; Peg-2 Stearate; Peg-20 Sorbitan Isostearate; Peg-22 Methyl Ether/Dodecyl Glycol Copolymer; Peg-25 Propylene Glycol Stearate; Peg-4 Dilaurate; Peg-4 Laurate; Peg-40 Castor Oil; Peg-40 Sorbitan Diisostearate; Peg-45/Dodecyl Glycol Copolymer; Peg-5 Oleate; Peg-50 Stearate; Peg-54 Hydrogenated Castor Oil; Peg-6 Isostearate; Peg-60 Castor Oil; Peg-60 Hydrogenated Castor Oil; Peg-7 Methyl Ether; Peg-75 Lanolin; Peg-8 Laurate; Peg-8 Stearate; Pegoxol 7 Stearate; Pentadecalactone; Pentaerythritol Cocoate; Pentasodium Pentetate; Pentetate Calcium Trisodium; Pentetic Acid; Peppermint Oil; Perflutren; Perfume 25677; Perfume Bouquet; Perfume E-1991; Perfume Gd 5604; Perfume Tana 90/42 Scba; Perfume W-1952-1; Petrolatum; Petrolatum, White; Petroleum Distillates; Phenol; Phenol, Liquefied; Phenonip; Phenoxyethanol; Phenylalanine; Phenylethyl Alcohol; Phenylmercuric Acetate; Phenylmercuric Nitrate; Phosphatidyl Glycerol, Egg; Phospholipid; Phospholipid, Egg; Phospholipon 90g; Phosphoric Acid; Pine Needle Oil (Pinus Sylvestris); Piperazine Hexahydrate; Plastibase-50w; Polacrilin; Polidronium Chloride; Poloxamer 124; Poloxamer 181; Poloxamer 182; Poloxamer 188; Poloxamer 237; Poloxamer-407; Poly(Bis(P-Carboxyphenoxy)Propane Anhydride):Sebacic Acid; Poly(Dimethylsiloxane/MethylvinylsiloxanelMethylhydrogensiloxane) Dimethylvinyl Or Dimethylhydroxy Or Trimethyl. Endblocked; Poly(D1-Lactic-Co-Glycolic Acid), (50:50; Poly(D1-Lactic-Co-Glycolic Acid), Ethyl Ester Terminated, (50:50; Polyacrylic Acid (250000 Mw); Polybutene (1400 Mw); Polycarbophil; Polyester; Polyester Polyamine Copolymer; Polyester Rayon; Polyethylene Glycol 1000; Polyethylene Glycol 1450; Polyethylene Glycol 1500; Polyethylene Glycol 1540; Polyethylene Glycol 200; Polyethylene Glycol 300; Polyethylene Glycol 300-1600; Polyethylene Glycol 3350; Polyethylene Glycol 400; Polyethylene Glycol 4000; Polyethylene Glycol 540; Polyethylene Glycol 600; Polyethylene Glycol 6000; Polyethylene Glycol 8000; Polyethylene Glycol 900; Polyethylene High Density Containing Ferric Oxide Black (<1%); Polyethylene Low Density Containing Barium Sulfate (20-24%); Polyethylene T; Polyethylene Terephthalates; Polyglactin; Polyglyceryl-3 Oleate; Polyglyceryl-4 Oleate; Polyhydroxyethyl Methacrylate; Polyisobutylene; Polyisobutylene (1100000 Mw); Polyisobutylene (35000 Mw); Polyisobutylene 178-236; Polyisobutylene 241-294; Polyisobutylene 35-39; Polyisobutylene Low Molecular Weight; Polyisobutylene Medium Molecular Weight; Polyisobutylene/Polybutene Adhesive; Polylactide; Polyols; Polyoxyethylene Polyoxypropylene 1800; Polyoxyethylene Alcohols; Polyoxyethylene Fatty Acid Esters; Polyoxyethylene Propylene; Polyoxyl 20 Cetostearyl Ether; Polyoxyl 35 Castor Oil; Polyoxyl 40 Hydrogenated Castor Oil; Polyoxyl 40 Stearate; Polyoxyl 400 Stearate; Polyoxyl 6 And Polyoxyl 32 Palmitostearate; Polyoxyl Distearate; Polyoxyl Glyceryl Stearate; Polyoxyl Lanolin; Polyoxyl Palmitate; Polyoxyl Stearate; Polypropylene; Polypropylene Glycol; Polyquaternium-10; Polyquaternium-7 (70/30 Acrylamide/Dadmac; Polysiloxane; Polysorbate 20; Polysorbate 40; Polysorbate 60; Polysorbate 65; Polysorbate 80; Polyurethane; Polyvinyl Acetate; Polyvinyl Alcohol; Polyvinyl Chloride; Polyvinyl Chloride-Polyvinyl Acetate Copolymer; Polyvinylpyridine; Poppy Seed Oil; Potash; Potassium Acetate; Potassium Alum; Potassium Bicarbonate; Potassium Bisulfite; Potassium Chloride; Potassium Citrate; Potassium Hydroxide; Potassium Metabisulfite; Potassium Phosphate, Dibasic; Potassium Phosphate, Monobasic; Potassium Soap; Potassium Sorbate; Povidone Acrylate Copolymer; Povidone Hydrogel; Povidone K17; Povidone K25; Povidone K29/32; Povidone K30; Povidone K90; Povidone K90f; Povidone/Eicosene Copolymer; Povidones; Ppg-12/Smdi Copolymer; Ppg-15 Stearyl Ether; Ppg-20 Methyl Glucose Ether Distearate; Ppg-26 Oleate; Product Wat; Proline; Promigen D; Promulgen G; Propane; Propellant A-46; Propyl Gallate; Propylene Carbonate; Propylene Glycol; Propylene Glycol Diacetate; Propylene Glycol Dicaprylate; Propylene Glycol Monolaurate; Propylene Glycol Monopalmitostearate; Propylene Glycol Palmitostearate; Propylene Glycol Ricinoleate; Propylene Glycol/Diazolidinyl Urea/Methylparaben/Propylparben; Propylparaben; Protamine Sulfate; Protein Hydrolysate; Pvm/Ma Copolymer; Quaternium-15; Quaternium-15 Cis-Form; Quaternium-52; Ra-2397; Ra-3011; Saccharin; Saccharin Sodium; Saccharin Sodium Anhydrous; Safflower Oil; Sd Alcohol 3a; Sd Alcohol 40; Sd Alcohol 40-2; Sd Alcohol 40b; Sepineo P 600; Serine; Sesame Oil; Shea Butter; Silastic Brand Medical Grade Tubing; Silastic Medical Adhesive, Silicone Type A; Silica, Dental; Silicon; Silicon Dioxide; Silicon Dioxide, Colloidal; Silicone; Silicone Adhesive 4102; Silicone Adhesive 4502; Silicone Adhesive Bio-Psa Q7-4201; Silicone Adhesive Bio-Psa Q7-4301; Silicone Emulsion; Silicone/Polyester Film Strip; Simethicone; Simethicone Emulsion; Sipon Ls 20np; Soda Ash; Sodium Acetate; Sodium Acetate Anhydrous; Sodium Alkyl Sulfate; Sodium Ascorbate; Sodium Benzoate; Sodium Bicarbonate; Sodium Bisulfate; Sodium Bi sulfite; Sodium Borate; Sodium Borate Decahydrate; Sodium Carbonate; Sodium Carbonate Decahydrate; Sodium Carbonate Monohydrate; Sodium Cetostearyl Sulfate; Sodium Chlorate; Sodium Chloride; Sodium Chloride Injection; Sodium Chloride Injection, Bacteriostatic; Sodium Cholesteryl Sulfate; Sodium Citrate; Sodium Cocoyl Sarcosinate; Sodium Desoxycholate; Sodium Dithionite; Sodium Dodecylbenzenesulfonate; Sodium Formaldehyde Sulfoxylate; Sodium Gluconate; Sodium Hydroxide; Sodium Hypochlorite; Sodium Iodide; Sodium Lactate; Sodium Lactate, L-; Sodium Laureth-2 Sulfate; Sodium Laureth-3 Sulfate; Sodium Laureth-5 Sulfate; Sodium Lauroyl Sarcosinate; Sodium Lauryl Sulfate; Sodium Lauryl Sulfoacetate; Sodium Metabisulfite; Sodium Nitrate; Sodium Phosphate; Sodium Phosphate Dihydrate; Sodium Phosphate, Dibasic; Sodium Phosphate, Dibasic, Anhydrous; Sodium Phosphate, Dibasic, Dihydrate; Sodium Phosphate, Dibasic, Dodecahydrate; Sodium Phosphate, Dibasic, Heptahydrate; Sodium Phosphate, Monobasic; Sodium Phosphate, Monobasic, Anhydrous; Sodium Phosphate, Monobasic, Dihydrate; Sodium Phosphate, Monobasic, Monohydrate; Sodium Polyacrylate (2500000 Mw); Sodium Pyrophosphate; Sodium Pyrrolidone Carboxylate; Sodium Starch Glycolate; Sodium Succinate Hexahydrate; Sodium Sulfate; Sodium Sulfate Anhydrous; Sodium Sulfate Decahydrate; Sodium Sulfite; Sodium Sulfosuccinated Undecyclenic Monoalkylolamide; Sodium Tartrate; Sodium Thioglycolate; Sodium Thiomalate; Sodium Thiosulfate; Sodium Thiosulfate Anhydrous; Sodium Trimetaphosphate; Sodium Xylenesulfonate; Somay 44; Sorbic Acid; Sorbitan; Sorbitan Isostearate; Sorbitan Monolaurate; Sorbitan Monooleate; Sorbitan Monopalmitate; Sorbitan Monostearate; Sorbitan Sesquioleate; Sorbitan Trioleate; Sorbitan Tri stearate; Sorbitol; Sorbitol Solution; Soybean Flour; Soybean Oil; Spearmint Oil; Spermaceti; Squalane; Stabilized Oxychloro Complex; Stannous 2-Ethylhexanoate; Stannous Chloride; Stannous Chloride Anhydrous; Stannous Fluoride; Stannous Tartrate; Starch; Starch 1500, Pregelatinized; Starch, Corn; Stearalkonium Chloride; Stearalkonium Hectorite/Propylene Carbonate; Stearamidoethyl Diethylamine; Steareth-10, Steareth-2; Steareth-20; Steareth-21; Steareth-40; Stearic Acid; Stearic Diethanolamide; Stearoxytrimethylsilane; Steartrimonium Hydrolyzed Animal Collagen; Stearyl Alcohol; Sterile Water For Inhalation; Styrene/Isoprene/Styrene Block Copolymer; Succimer; Succinic Acid; Sucralose; Sucrose; Sucrose Distearate; Sucrose Polyesters; Sulfacetamide Sodium; Sulfobutylether .Beta.-Cyclodextrin; Sulfur Dioxide; Sulfuric Acid; Sulfurous Acid; Surfactol Qs; Tagatose, D-; Talc; Tall Oil; Tallow Glycerides; Tartaric Acid; Tartaric Acid, D1-; Tenox; Tenox-2; Tert-Butyl Alcohol; Tert-Butyl Hydroperoxide; Tert-Butylhydroquinone; Tetrakis(2-Methoxyisobutylisocyanide)Copper(f) Tetrafluoroborate; Tetrapropyl Orthosilicate; Tetrofosmin; Theophylline; Thimerosal; Threonine; Thymol; Tin; Titanium Dioxide; Tocopherol; Tocophersolan; Total parenteral nutrition, lipid emulsion; Triacetin; Tricaprylin; Trichloromonofluoromethane; Trideceth-10; Triethanolamine Lauryl Sulfate; Trifluoroacetic Acid; Triglycerides, Medium Chain; Trihydroxystearin; Trilaneth-4 Phosphate; Trilaureth-4 Phosphate; Trisodium Citrate Dihydrate; Tri sodium Hedta; Triton 720; Triton X-200; Trolamine; Tromantadine; Tromethamine (TRIS); Tryptophan; Tyloxapol; Tyrosine; Undecylenic Acid; Union 76 Amsco-Res 6038; Urea; Valine; Vegetable Oil; Vegetable Oil Glyceride, Hydrogenated; Vegetable Oil, Hydrogenated; Versetamide; Viscarin; Viscose/Cotton; Vitamin E; Wax, Emulsifying; Wecobee Fs; White Ceresin Wax; White Wax; Xanthan Gum; Zinc; Zinc Acetate; Zinc Carbonate; Zinc Chloride; and Zinc Oxide.

Pharmaceutical composition formulations of viral particles disclosed herein may include cations or anions. In some embodiments, the formulations include metal cations such as, but not limited to, Zn2+, Ca2+, Cu2+, Mn2+, Mg+ and combinations thereof. As a non-limiting example, formulations may include polymers and complexes with a metal cation (See e.g., U.S. Pat. Nos. 6,265,389 and 6,555,525, each of which is herein incorporated by reference in its entirety).

Formulations of the disclosure may also include one or more pharmaceutically acceptable salts. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and airline cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids.

Solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(114)-pyrimidinone (DMPU), acetonitrile (ACCT), propylene glycol, ethyl acetate, benzyl alcohol, 2-pyrrolidone, benzyl benzoate, and the like. When water is the solvent, the solvate is referred to as a “hydrate.”

### III. Administration and Dosing

### Administration

The viral particles of the present disclosure may be administered by any delivery route which results in a therapeutically effective outcome. These include, but are not limited to, enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into brain tissue), intraperitoneal (infusion or injection into the peritoneum), intravesical infusion, intravitreal (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis, and spinal.

In some embodiments, compositions may be administered in a way which allows them to cross the blood-brain barrier, vascular barrier, or other epithelial barrier. The viral particles of the present disclosure may be administered in any suitable form, either as a liquid solution or suspension, as a solid form suitable for liquid solution or suspension in a liquid solution. The viral particles may be formulated with any appropriate and pharmaceutically acceptable excipient.

In some embodiments, the viral particles of the present disclosure may be delivered to a subject via a single route administration.

In some embodiments, the viral particles of the present disclosure may be delivered to a subject via a multi-site route of administration. A subject may be administered at 2, 3, 4, 5, or more than 5 sites.

In some embodiments, a subject may be administered the viral particles of the present disclosure using a bolus infusion.

In some embodiments, a subject may be administered the viral particles of the present disclosure using sustained delivery over a period of minutes, hours, or days. The infusion rate may be changed depending on the subject, distribution, formulation or another delivery parameter.

In some embodiments, the viral particles of the present disclosure may be delivered by intramuscular delivery route. (See, e.g., U.S. Pat. No. 6,506,379; the content of which is incorporated herein by reference in its entirety). Non-limiting examples of intramuscular administration include an intravenous injection or a subcutaneous injection.

In some embodiments, the viral particles of the present disclosure may be delivered by oral administration. Non-limiting examples of oral administration include a digestive tract administration and a buccal administration.

In some embodiments, the viral particles of the present disclosure may be delivered by intraocular delivery route. A non-limiting example of intraocular administration include an intravitreal injection.

In some embodiments, the viral particles of the present disclosure may be delivered by intranasal delivery route. Non-limiting examples of intranasal delivery include administration of nasal drops or nasal sprays.

In some embodiments, the viral particles that may be administered to a subject by peripheral injections. Non-limiting examples of peripheral injections include intraperitoneal, intramuscular, intravenous, conjunctival, or joint injection. It was disclosed in the art that the peripheral administration of AAV vectors can be transported to the central nervous system, for example, to the motor neurons (e.g., U.S. Patent Publication Nos. US20100240739 and US20100130594; the content of each of which is incorporated herein by reference in their entirety).

In some embodiments, the viral particles may be delivered by injection into the CSF pathway. Non-limiting examples of delivery to the CSF pathway include intrathecal and intracerebroventricular administration.

In some embodiments, the viral particles may be delivered by systemic delivery. As a non-limiting example, the systemic delivery may be by intravascular administration.

In some embodiments, the viral particles of the present disclosure may be administered to a subject by intracranial delivery (See, e.g., U.S. Pat. No. 8,119,611; the content of which is incorporated herein by reference in its entirety).

In some embodiments, the viral particles of the present disclosure may be administered to a subject by intraparenchymal administration.

In some embodiments, the viral particles of the present disclosure may be administered to a subject by intramuscular administration.

In some embodiments, the viral particles of the present disclosure are administered to a subject and transduce muscle of a subject. As a non-limiting example, the viral particles are administered by intramuscular administration.

In some embodiments, the viral particles of the present disclosure may be administered to a subject by intravenous administration.

In some embodiments, the viral particles of the present disclosure may be administered to a subject by subcutaneous administration.

In some embodiments, the viral particles of the present disclosure may be administered to a subject by topical administration.

In some embodiments, the viral particles may be delivered by direct injection into the brain. As a nonlimiting example, the brain delivery may be by intrastriatal administration.

In some embodiments, the viral particles may be delivered by more than one route of administration. As nonlimiting examples of combination administrations, viral particles may be delivered by intrathecal and intracerebroventricular, or by intravenous and intraparenchymal administration.

### Parenteral and Injectable Administration

In some embodiments, pharmaceutical compositions, viral particles of the present disclosure may be administered parenterally. Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof. In other embodiments, surfactants are included such as hydroxypropylcellulose.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### Rectal and Vaginal Administration

In some embodiments, pharmaceutical compositions, viral particles of the present disclosure may be administered rectally and/or vaginally. Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

### Oral Administration

In some embodiments, pharmaceutical compositions, viral particles of the present disclosure may be administered orally. Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

### Topical or Transdermal Administration

As described herein, pharmaceutical compositions, viral particles of the present disclosure may be formulated for administration topically. The skin may be an ideal target site for delivery as it is readily accessible. Three routes are commonly considered to deliver pharmaceutical compositions, viral particles of the present disclosure to the skin: (i) topical application (e.g. for local/regional treatment and/or cosmetic applications); (ii) intradermal injection (e.g. for local/regional treatment and/or cosmetic applications); and (iii) systemic delivery (e for treatment of dermatologic diseases that affect both cutaneous and extracutaneous regions). Pharmaceutical compositions, viral particles of the present disclosure can be delivered to the skin by several different approaches known in the art.

In some embodiments, the disclosure provides for a variety of dressings (e.g., wound dressings) or bandages (e.g., adhesive bandages) for conveniently and/or effectively carrying out methods of the present disclosure. Typically dressing or bandages may comprise sufficient amounts of pharmaceutical compositions, viral particles of the present disclosure described herein to allow users to perform multiple treatments.

Dosage forms for topical and/or transdermal administration may include ointments, pastes, creams, lotions, gels, powders, solutions, sprays, inhalants and/or patches. Generally, active ingredients are admixed under sterile conditions with pharmaceutically acceptable excipients and/or any needed preservatives and/or buffers. Additionally, the present disclosure contemplates the use of transdermal patches, which often have the added advantage of providing controlled delivery of pharmaceutical compositions, viral particles of the present disclosure to the body. Such dosage forms may be prepared, for example, by dissolving and/or dispensing pharmaceutical compositions, viral particles in the proper medium. Alternatively, or additionally, rates may be controlled by either providing rate controlling membranes and/or by dispersing pharmaceutical compositions, viral particles in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 1% (to about 10% (w/w) active ingredient, although the concentration of active ingredient may be as high as the solubility limit of the active ingredient in the solvent. Formulations for topical administration may further comprise one or more of the additional ingredients described herein.

### Depot Administration

As described herein, in some embodiments, pharmaceutical compositions, viral particles of the present disclosure are formulated in depots for extended release. Generally, specific organs or tissues (“target tissues”) are targeted for administration.

In some aspects of the disclosure, pharmaceutical compositions, viral particles of the present disclosure are spatially retained within or proximal to target tissues. Provided are methods of providing pharmaceutical compositions, viral particles, to target tissues of mammalian subjects by contacting target tissues (which comprise one or more target cells) with pharmaceutical compositions, viral particles, under conditions such that they are substantially retained in target tissues, meaning that at least 10, 20, 30, 40, 50, 60, 70, 80, 85, 90, 95, 96, 97, 98, 99, 99.9, 99.99 or greater than 99,99% of the composition is retained in the target tissues. Advantageously, retention is determined by measuring the amount of pharmaceutical compositions, viral particles, that enter one or more target cells. For example, at least 1%, 5%, 10%, 20%, 30%, 40%, 50%, 60%, 70%, 80%, 85%, 90%, 95%, 96%, 97%, 98%, 99%, 99.9%, 99.99%, or greater than 99.99% of pharmaceutical compositions, viral particles, administered to subjects are present intracellularly at a period of time following administration. For example, intramuscular injection to mammalian subjects may be performed using aqueous compositions comprising pharmaceutical compositions, viral particles of the present disclosure and one or more transfection reagents, and retention is determined by measuring the amount of pharmaceutical compositions, viral particles, present in muscle cells.

Certain aspects of the disclosure are directed to methods of providing pharmaceutical compositions, viral particles of the present disclosure to a target tissues of mammalian subjects, by contacting target tissues (comprising one or more target cells) with pharmaceutical compositions, viral particles under conditions such that they are substantially retained in such target tissues. Pharmaceutical compositions, viral particles comprise enough active ingredient such that the effect of interest is produced in at least one target cell. In some embodiments, pharmaceutical compositions, viral particles generally comprise one or more cell penetration agents, although “naked” formulations (such as without cell penetration agents or other agents) are also contemplated, with or without pharmaceutically acceptable carriers.

### Pulmonary Administration

In some embodiments, pharmaceutical compositions, viral particles of the present disclosure may be prepared, packaged, and/or sold in formulations suitable for pulmonary administration. In some embodiments, such administration is via the buccal cavity. In some embodiments, formulations may comprise dry particles comprising active ingredients. In such embodiments, dry particles may have a diameter in the range from about 0.5 nm to about 7 nm or from about 1 nm to about 6 nm. In some embodiments, formulations may be in the form of dry powders for administration using devices comprising dry powder reservoirs to which streams of propellant may be directed to disperse such powder. In some embodiments, self-propelling solvent/powder dispensing containers may be used. In such embodiments, active ingredients may be dissolved and/or suspended in low-boiling propellant in sealed containers. Such powders may comprise particles wherein at least 98% of the particles by weight have diameters greater than 0.5 nm and at least 95% of the particles by number have diameters less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally, propellants may constitute 50% to 99.9% (w/w) of the composition, and active ingredient may constitute 0.1% to 20% (w/w) of the composition. Propellants may further comprise additional ingredients such as liquid non-ionic and/or solid anionic surfactant and/or solid diluent (which may have particle sizes of the same order as particles comprising active ingredients).

Pharmaceutical compositions formulated for pulmonary delivery may provide active ingredients in the form of droplets of solution and/or suspension. Such formulations may be prepared, packaged, and/or sold as aqueous and/or dilute alcoholic solutions and/or suspensions, optionally sterile, comprising active ingredients, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

### Intranasal, Nasal and Buccal Administration

In some embodiments, pharmaceutical compositions, viral particles of the present disclosure may be administered nasally and/or intranasal. In some embodiments, formulations described herein useful for pulmonary delivery may also be useful for intranasal delivery. In some embodiments, formulations for intranasal administration comprise a coarse powder comprising the active ingredient and having an average particle from about 0.2 μm to 500 μm. Such formulations are administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise powders and/or an aerosolized and/or atomized solutions and/or suspensions comprising active ingredients. Such powdered, aerosolized, and/or aerosolized formulations, when dispersed, may comprise average particle and/or droplet sizes in the range of from about 0.1 nm to about 200 nm, and may further comprise one or more of any additional ingredients described herein.

### Ophthalmic or Otic Administration

In some embodiments, pharmaceutical compositions, viral particles of the present disclosure may be prepared, packaged, and/or sold in formulations suitable for ophthalmic and/or otic administration. Such formulations may, for example, be in the form of eye and/or ear drops including, for example, a 0.1/1.0% (w/w) solution and/or suspension of the active ingredient in aqueous and/or oily liquid excipients. Such drops may further comprise buffering agents, salts, and/or one or more other of any additional ingredients described herein. Other ophthalmically-administrable formulations which are useful include those which comprise active ingredients in microcrystalline form and/or in liposomal preparations. Subretinal inserts may also be used as forms of administration.

### Delivery

In some embodiments, the viral particles or pharmaceutical compositions of the present disclosure may be administered or delivered using the methods for treatment of disease described in U.S. Pat. No. 8,999,948, or International Publication No. WO2014178863, the contents of which are herein incorporated by reference in their entirety.

In some embodiments, the viral particles or pharmaceutical compositions of the present disclosure may be administered or delivered using the methods for delivering gene therapy in Alzheimer's Disease or other neurodegenerative conditions as described in US Application No. 20150126590, the contents of which are herein incorporated by reference in their entirety.

In some embodiments, the viral particles or pharmaceutical compositions of the present disclosure may be administered or delivered using the methods for delivery of a CNS gene therapy as described in U.S. Pat. Nos. 6,436,708, and 8,946,152, and International Publication No. WO2015168666, the contents of which are herein incorporated by reference in their entirety.

In some embodiments, the viral particle or pharmaceutical compositions of the present disclosure may be administered or delivered using the methods for delivering proteins using AAV vectors described in European Patent Application No. EP2678433, the contents of which are herein incorporated by reference in their entirety.

In some embodiments, the viral particle or pharmaceutical compositions of the present disclosure may be administered or delivered using the methods for delivering DNA to the bloodstream described in U.S. Pat. No. 6,211,163, the contents of which are herein incorporated by reference in their entirety.

In some embodiments, the viral particle or pharmaceutical compositions of the present disclosure may be administered or delivered using the methods for delivering a payload to the central nervous system described in U.S. Pat. No. 7,588,757, the contents of which are herein incorporated by reference in their entirety.

In some embodiments, the viral particle or pharmaceutical compositions of the present disclosure may be administered or delivered using the methods for delivering a payload described in U.S. Pat. No. 8,283,151, the contents of which are herein incorporated by reference in their entirety.

In some embodiments, the viral particle or pharmaceutical compositions of the present disclosure may be administered or delivered using the methods for delivering a payload using a glutamic acid decarboxylase (GAD) delivery vector described in International Patent Publication No. WO2001089583, the contents of which are herein incorporated by reference in their entirety.

In some embodiments, the viral particle or pharmaceutical compositions of the present disclosure may be administered or delivered using the methods for delivering a payload to neural cells described in International Patent Publication No. WO2012057363, the contents of which are herein incorporated by reference in their entirety.

### Delivery to Cells

The present disclosure provides a method of delivering to a cell or tissue any of the above-described viral particles, comprising contacting the cell or tissue with said viral particle or contacting the cell or tissue with a formulation comprising said viral particle, or contacting the cell or tissue with any of the described compositions, including pharmaceutical compositions. The method of delivering the viral particle to a cell or tissue can be accomplished in vitro, ex vivo, or in vivo.

### Delivery to Subjects

The present disclosure additionally provides a method of delivering to a subject, including a mammalian subject, any of the above-described viral particles comprising administering to the subject said viral particle, or administering to the subject a formulation comprising said viral particle, or administering to the subject any of the described compositions, including pharmaceutical compositions.

### Dose and Regimen

The present disclosure provides methods of administering viral particles in accordance with the disclosure to a subject in need thereof. The pharmaceutical, diagnostic, or prophylactic viral particles and compositions of the present disclosure may be administered to a subject using any amount and any route of administration effective for preventing, treating, managing, or diagnosing diseases, disorders and/or conditions. The exact amount required will vary from subject to subject, depending on the species, age, and general condition of the subject, the severity of the disease, the particular composition, its mode of administration, its mode of activity, and the like. The subject may be a human, a mammal, or an animal. Compositions in accordance with the disclosure are typically formulated in unit dosage form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate diagnostic dose level for any particular individual will depend upon a variety of factors including the disorder being treated and the severity of the disorder; the activity of the specific payload employed; the specific composition employed; the age, body weight, general health, sex and diet of the patient; the time of administration, route of administration, and rate of excretion of the specific viral particle employed; the duration of the treatment; drugs used in combination or coincidental with the specific viral particle employed; and like factors well known in the medical arts.

In certain embodiments, viral particle pharmaceutical compositions in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect, it will be understood that the above dosing concentrations may be converted to vg or viral genomes per kg or into total viral genomes administered by one of skill in the art.

In certain embodiments, viral particle pharmaceutical compositions in accordance with the present disclosure may be administered at about 10 to about 600 μl/site, 50 to about 500 μl/site, 100 to about 400 μl/site, 120 to about 300 μl/site, 140 to about 200 μl/site, about 160 1,11/site. As non-limiting examples, viral particles may be administered at 50 μl/site and/or 150 μl/site.

The desired dosage of the viral particles of the present disclosure may be delivered only once, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of “single unit dose” or total daily dose into two or more doses, e.g., two or more administrations of the “single unit dose”. As used herein, a “single unit dose” is a dose of any therapeutic administered in one dose/at one time/single route/single point of contact, i.e., single administration event.

The desired dosage of the viral particles of the present disclosure may be administered as a “pulse dose” or as a “continuous flow”. As used herein, a “pulse dose” is a series of single unit doses of any therapeutic administered with a set frequency over a period of time. As used herein, a “continuous flow” is a dose of therapeutic administered continuously for a period of time in a single route/single point of contact, i.e., continuous administration event. A total daily dose, an amount given or prescribed in 24-hour period, may be administered by any of these methods, or as a combination of these methods, or by any other methods suitable for a pharmaceutical administration.

In some embodiments, delivery of the viral particles of the present disclosure to a subject provides neutralizing activity to a subject. The neutralizing activity can be for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years.

In some embodiments, delivery of the viral particles of the present disclosure results in minimal serious adverse events (SAES) as a result of the delivery of the viral particles.

In some embodiments, delivery of viral particles to cells of the central nervous system (e.g., parenchyma) may comprise a total dose between about 1×10⁶ VG and about 1×10 6 VG. In some embodiments, delivery may comprise a total dose of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1.9×10¹⁰, 2×10¹⁰, 3×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.5×10¹¹, 3×10¹¹, 4×10¹¹, 5×10¹¹, 6×10¹¹, 7×10¹¹, 8×10¹¹, 9×10¹¹, 11×10¹², 2×10¹², 3×10¹², 4×10¹², 5×10¹², 6×10¹², 7×10¹², 8×10¹², 9×10¹¹, 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG. As a non-limiting example, the total dose is 1×10¹³ VG. As another non-limiting example, the total dose is 2.1×10¹² VG.

In some embodiments, delivery of viral particles to cells of the central nervous system (e.g., parenchyma) may comprise a composition concentration between about 1×10⁶ VG/mL and about 1×10¹⁶ VG/ML. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10 9 9×10⁹, 1.9×10¹⁰, 2×10¹⁰, 3×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.5×10¹¹, 3×10¹¹, 4×10¹¹, 5×10¹¹, 6×10¹¹, 7×10¹¹, 8×10¹¹, 9×10¹¹, 11×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL. In some embodiments, the delivery comprises a composition concentration of 1×10¹³ VG/mL. In some embodiments, the delivery comprises a composition concentration of 2×10¹² VG/mL.

### Combinations

The viral particles may be used in combination with one or more other therapeutic, prophylactic, research or diagnostic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, research, or diagnostic compositions in combination with agents that may improve their bioavailability, reduce and/or modify their metabolism, inhibit their excretion, and/or modify their distribution within the body.

### Measurement of Expression

Expression of payloads from viral genomes may be determined using various methods known in the art such as, but not limited to immunochemistry (e.g., IHC), in situ hybridization (ISH), enzyme-linked immunosorbent assay (ELISA), affinity ELISA, ELISPOT, flow cytometry, immunocytology, surface plasmon resonance analysis, kinetic exclusion assay, liquid chromatography-mass spectrometry (LCMS), high-performance liquid chromatography (HPLC), BCA assay, immunoelectrophoresis, Western blot, SDS-PAGE, protein immunoprecipitation, and/or PCR.

### Bioavailability

The viral particles, when formulated into a composition with a delivery agent as described herein, can exhibit an increase in bioavailability as compared to a composition lacking a delivery agent as described herein. As used herein, the term “bioavailability” refers to the systemic availability of a given amount of viral particle or expressed payload administered to a mammal. Bioavailability can be assessed by measuring the area under the curve (AUC) or the maximum serum or plasma concentration (Cₘₐₓ) of the composition following. AUC is a determination of the area under the curve plotting the serum or plasma concentration of a compound (e.g., viral particles or expressed payloads) along the ordinate (Y-axis) against time along the abscissa (X-axis). Generally, the AUC for a particular compound can be calculated using methods known to those of ordinary skill in the art and as described in G. S. Banker, Modern Pharmaceutics, Drugs and the Pharmaceutical Sciences, v. 72, Marcel Dekker, New York, Inc., 1996, the contents of which are herein incorporated by reference in its entirety.

The Cₘₐₓ value is the maximum concentration of the viral particle or expressed payload achieved in the serum or plasma of a mammal following administration of the viral particle to the mammal. The Cₘₐₓ value of can be measured using methods known to those of ordinary skill in the art. The phrases “increasing bioavailability” or “improving the pharmacokinetics,” as used herein mean that the systemic availability of a first viral particle or expressed payload, measured as AUC, Cₘₐₓ, or Cₘᵢₙ in a mammal is greater, when co-administered with a delivery agent as described herein, than when such co-administration does not take place. In some embodiments, the bioavailability can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Therapeutic Window

As used herein “therapeutic window” refers to the range of plasma concentrations, or the range of levels of therapeutically active substance at the site of action, with a high probability of eliciting a therapeutic effect. In some embodiments, the therapeutic window of the viral particle as described herein can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Volume of Distribution

As used herein, the term “volume of distribution” refers to the fluid volume that would be required to contain the total amount of the drug in the body at the same concentration as in the blood or plasma: V_{dist} equals the amount of drug in the body/concentration of drug in blood or plasma. For example, for a 10 mg dose and a plasma concentration of 10 mg/L, the volume of distribution would be 1 liter. The volume of distribution reflects the extent to which the drug is present in the extravascular tissue. A large volume of distribution reflects the tendency of a compound to bind to the tissue components compared with plasma protein binding. In a clinical setting, V_{dist} can be used to determine a loading dose to achieve a steady state concentration. In some embodiments, the volume of distribution of the viral particles as described herein can decrease at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%.

### Biological Effect

In some embodiments, the biological effect of the viral particles delivered to the animals may be categorized by analyzing the payload expression in the animals. The payload expression may be determined from analyzing a biological sample collected from a mammal administered the viral particles of the present disclosure. For example, a protein expression of 50-200 pg/ml for the protein encoded by the viral particles delivered to the mammal may be seen as a therapeutically effective amount of protein in the mammal.

### IV. Methods and Uses of the Compositions of the Disclosure

The present disclosure provides a method for treating a disease, disorder and/or condition in a mammalian subject, including a human subject, comprising administering to the subject any of the viral particles described herein or administering to the subject any of the described compositions, including pharmaceutical compositions, described herein.

In some embodiments, the viral particles of the present disclosure are administered to a subject prophylactically.

In some embodiments, the viral particles of the present disclosure are administered to a subject having at least one of the diseases described herein.

In some embodiments, the viral particles of the present disclosure are administered to a subject to treat a disease or disorder described herein. The subject may have the disease or disorder or may be at-risk to developing the disease or disorder.

In some embodiments, the viral particles of the present disclosure are part of an active immunization strategy to protect against diseases and disorders. In an active immunization strategy, a vaccine or viral particles are administered to a subject to prevent an infectious disease by activating the subject's production of antibodies that can fight off invading bacteria or viruses.

In some embodiments, the viral particles of the present disclosure are part of a passive immunization strategy. In a passive immunization strategy, antibodies against a particular infectious agent are given directly to the subject.

### Diseases and Toxins

Various infectious diseases may be treated with pharmaceutical compositions, viral particles, of the present disclosure. As used herein, the term “infectious disease” refers to any disorders caused by organisms such as bacteria, viruses, fungi or parasites. As a non-limiting example, the infectious disease may be Acute bacterial rhinosinusitis, 14-day measles, Acne, Acrodermatitis chronica atrophicans (ACA)-(late skin manifestation of latent Lyme disease), Acute hemorrhagic conjunctivitis, Acute hemorrhagic cystitis, Acute rhinosinusitis, Adult T-cell Leukemia-Lymphoma (ATLL), African Sleeping Sickness, AIDS (Acquired Immunodeficiency Syndrome), Alveolar hydatid, Amebiasis, Amebic meningoencephalitis, Anaplasmosis, Anthrax, Arboviral or parainfectious, Ascariasis (Roundworm infections), Aseptic meningitis, Athlete's foot (Tinea pedis), Australian tick typhus, Avian Influenza, Babesiosis, Bacillary angiomatosis, Bacterial meningitis, Bacterial vaginosis, Balanitis, Balantidiasis, Bang's disease, Barmah Forest virus infection, Bartonellosis (Verruga peruana; Carrion's disease; Oroya fever), Bat Lyssavirus Infection, Bay sore (Chiclero's ulcer), Baylisascaris infection (Racoon roundworm infection), Beaver fever, Beef tapeworm, Bejel (endemic syphilis), Biphasic meningoencephalitis, Black Bane, Black death, Black piedra, Blackwater Fever, Blastomycosis, Blennorrhea of the newborn, Blepharitis, Boils, Bornholm disease (pleurodynia), Borrelia miyamotoi Disease, Botulism, Boutonneuse fever, Brazilian purpuric fever, Break Bone fever, Brill, Bronchiolitis, Bronchitis, Brucellosis (Bang's disease), Bubonic plague, Bullous impetigo, Burkholderia mallei (Glanders), Burkholderia pseudomallei (Melioidosis), Buruli ulcers (also Mycoburuli ulcers), Busse, Busse-Buschke disease (Cryptococcosis), California group encephalitis, Campylobacteriosis, Candidiasis, Canefield fever (Canicola fever; 7-day fever; Weil's disease; leptospirosis; canefield fever), Canicola fever, Capillariasis, Carate, Carbapenem-resistant Enterobacteriaceae (CRE), Carbuncle, Carrion's disease, Cat Scratch fever, Cave disease, Central Asian hemorrhagic fever, Central European tick, Cervical cancer, Chagas disease, Chancroid (Soft chancre), Chicago disease, Chickenpox (Varicella), Chiclero's ulcer, Chikungunya fever, Chlamydial infection, Cholera, Chromoblastomycosis, Ciguatera, Clap, Clonorchiasis (Liver fluke infection), Clostridium Difficile Infection, Clostridium Perfringens (Epsilon Toxin), Coccidioidomycosis fungal infection (Valley fever; desert rheumatism), Coenurosis, Colorado tick fever, Condyloma accuminata, Condyloma accuminata(Warts), Condyloma lata, Congo fever, Congo hemorrhagic fever virus, Conjunctivitis, cowpox, Crabs, Crimean, Croup, Cryptococcosis, Cryptosporidiosis (Crypto), Cutaneous Larval Migrans, Cyclosporiasis, Cystic hydatid, Cysticercosis, Cystitis, Czechoslovak tick, D68 (EV-D68), Dacryocytitis, Dandy fever, Darling's Disease, Deer fly fever, Dengue fever (1, 2, 3 and 4), Desert rheumatism, Devil's grip, Diphasic milk fever, Diphtheria, Disseminated Intravascular Coagulation, Dog tapeworm, Donovanosis, Donovanosis (Granuloma inguinale), Dracontiasis, Dracunculosis, Duke's disease, Dum Dum Disease, Durand-Nicholas-Favre disease, Dwarf tapeworm, E. Coli infection (E. Coli), Eastern equine encephalitis, Ebola Hemorrhagic Fever (Ebola virus disease EVD), Ectothrix, Ehrlichiosis (Sennetsu fever), Encephalitis, Endemic Relapsing fever, Endemic syphilis, Endophthalmitis, Endothrix, Enterobiasis (Pinworm infection), Enterotoxins B Poisoning (Staph Food Poisoning), Enterovirus Infection, Epidemic Keratoconjunctivitis, Epidemic Relapsing fever, Epidemic typhus, Epiglottitis, Erysipelis, Erysipeloid (Erysipelothricosis), Erythema chronicum migrans, Erythema infectiosum, Erythema marginatum, Erythema multiforme, Erythema nodosum, Erythema nodosum leprosum, Erythrasma, Espundia, Eumycotic mycetoma, European blastomycosis, Exanthem subitum (Sixth disease), Eyeworm, Far Eastern tick, Fascioliasis, Fievre boutonneuse (Tick typhus), Fifth Disease (erythema infectiosum), Filatow-Dukes' Disease (Scalded Skin Syndrome; Ritter's Disease), Fish tapeworm, Fitz-Hugh-Curtis syndrome—Perihepatitis, Flinders Island Spotted Fever, Flu (Influenza), Folliculitis, Four Corners Disease, Four Corners Disease (Human Pulmonary Syndrome (HPS)), Frambesia, Francis disease, Furunculosis, Gas gangrene, Gastroenteritis, Genital Herpes, Genital Warts, German measles, Gerstmann-Straussler-Scheinker (GSS), Giardiasis, Gilchrist's disease, Gingivitis, Gingivostomatitis, Glanders, Glandular fever (infectious mononucleosis), Gnathostomiasis, Gonococcal Infection (Gonorrhea), Gonorrhea, Granuloma inguinale (Donovanosis), Guinea Worm, Haemophilus Influenza disease, Hamburger disease, Hansen's disease-leprosy, Hantaan disease, Hantaan-Korean hemorrhagic fever, Hantavirus Pulmonary Syndrome, Hantavirus Pulmonary Syndrome (HPS), Hard chancre, Hard measles, Haverhill fever—Rat bite fever, Head and Body Lice, Heartland fever, Helicobacterosis, Hemolytic Uremic Syndrome (WS), Hepatitis A, Hepatitis B, Hepatitis C, Hepatitis D, Hepatitis E, Herpangina, Herpes-genital, Herpes labialis, Herpes-neonatal, Hidradenitis, Histoplasmosis, Histoplasmosis infection (Histoplasmosis), His-Werner disease, HIV infection, Hookworm infections, Hordeola, Hordeola (Stye), HTLV, HTLV-associated myelopathy (HAM), Human granulocytic ehrlichiosis, Human monocytic ehrlichiosis, Human Papillomavirus (HPV), Human Pulmonary Syndrome, Hydatid cyst, Hydrophobia, Impetigo, Including congenital (German Measles), Inclusion conjunctivitis, Inclusion conjunctivitis-Swimming Pool conjunctivitis-Pannus, Infantile diarrhea, Infectious Mononucleosis, Infectious myocarditis, Infectious pericarditis, influenza, isosporiasis, Israeli spotted fever, Japanese Encephalitis, Jock itch, Jorge Lobo disease lobomycosis, Jungle yellow fever, Junin Argentinian hemorrhagic fever, Kala Azar, Kaposi's sarcoma, Keloidal blastomycosis, Keratoconjunctivitis, Kuru, Kyasanur forest disease, LaCrosse encephalitis, Lassa hemorrhagic fever, Legionellosis (Legionnaires Disease), Legionnaire's pneumonia, Lemierre's Syndrome (Postanginal septicemia), Lemming fever, Leprosy, Leptospirosis (Nanukayami fever; Weirs disease), Listeriosis (Listeria), Liver fluke infection, Lobo's mycosis, Lockjaw, Loiasis, Louping Ill, Ludwig's angina, Lung fluke infection, Lung fluke infection (Paragonimiasis), Lyme disease, Lymphogranuloma venereum infection (LGV), Machupo Bolivian hemorrhagic fever, Madura foot, Mal del pinto, Malaria, Malignant pustule, Malta fever, Marburg hemorrhagic fever, Masters disease, Maternal Sepsis (Puerperal fever), Measles, Mediterranean spotted fever, Melioidosis (Whitmore's disease), Meningitis, Meningococcal Disease, MERS, Milker's nodule, Molluscum contagiosum, Moniliasis, monkeypox, Mononucleosis, Mononucleosis-like syndrome, Montezuma's Revenge, Morbilli, MRSA (methicillin-resistant Staphylococcus aureus) infection, Mucormycosis-Zygomycosis, Multiple Organ Dysfunction Syndrome or MODS, Multiple-system atrophy (MSA), Mumps, Murine typhus, Murray Valley Encephalitis (MVE), Mycoburuli ulcers, Mycoburuli ulcers-Buruli ulcers, Mycotic vulvovaginitis, Myositis, Nanukayami fever, Necrotizing fasciitis, Necrotizing fasciitis-Type 1, Necrotizing fasciitis-Type 2, Negishi, New world spotted fever, Nocardiosis, Nongonococcal urethritis, Non-Polio (Non-Polio Enterovirus), Norovirus infection, North American blastomycosis, North Asian tick typhus, Norwalk virus infection, Norwegian itch, O'Hara disease, Omsk hemorrhagic fever, Onchoceriasis, Onychomycosis, Opisthorchiasis, Opthalmia neonatorium, Oral hairy leukoplakia, Off, Oriental Sore, Oriental Spotted Fever, Ornithosis (Parrot fever; Psittacosis), Oroya fever, Otitis externa, Otitis media, Pannus, Paracoccidioidomycosis, Paragonimiasis, Paralytic Shellfish Poisoning (Paralytic Shellfish Poisoning), Paronychia (Whitlow), Parotitis, PCP pneumonia, Pediculosis, Peliosis hepatica, Pelvic Inflammatory Disease, Pertussis (also called Whooping cough), Phaeohyphomycosis, Pharyngoconjunctival fever, Piedra (White Piedra), Piedra (Black Piedra), Pigbel, Pink eye conjunctivitis, Pinta, Pinworm infection, Pitted Keratolysis, Pityriasis versicolor (Tinea versicolor), Plague; Bubonic, Pleurodynia, Pneumococcal Disease, Pneumocystosis, Pneumonia, Pneumonic (Plague), Polio or Poliomyelitis, Polycystic hydatid, Pontiac fever, Pork tapeworm, Posada-Wernicke disease, Postanginal septicemia, Powassan, Progressive multifocal leukencephalopathy, Progressive Rubella Panencephalitis, Prostatitis, Pseudomembranous colitis, Psittacosis, Puerperal fever, Pustular Rash diseases (Small pox), Pyelonephritis, Pylephlebitis, Q-Fever, Quinsy, Quintana fever (5-day fever), Rabbit fever, Rabies, Racoon roundworm infection, Rat bite fever, Rat tapeworm, Reiter Syndrome, Relapsing fever, Respiratory syncytial virus (RSV) infection, Rheumatic fever, Rhodotorulosis, Ricin Poisoning, Rickettsialpox, Rickettsiosis, Rift Valley Fever, Ringworm, Ritter's Disease, River Blindness, Rocky Mountain spotted fever, Rose Handler's disease (Sporotrichosis), Rose rash of infants, Roseola, Ross River fever, Rotavirus infection, Roundworm infections, Rubella, Rubeola, Russian spring, Salmonellosis gastroenteritis, San Joaquin Valley fever, Sao Paulo Encephalitis, Sao Paulo fever, SARS, Scabies Infestation (Scabies) (Norwegian itch), Scalded Skin Syndrome, Scarlet fever (Scarlatina), Schistosomiasis, Scombroid, Scrub typhus, Sennetsu fever, Sepsis (Septic shock), Severe Acute Respiratory Syndrome, Severe Acute Respiratory Syndrome (SARS), Shiga Toxigenic Escherichia coli (STECNTEC), Shigellosis gastroenteritis (Shigella), Shinbone fever, Shingles, Shipping fever, Siberian tick typhus, Sinusitis, Sixth disease, Slapped cheek disease, Sleeping sickness, Smallpox (Variola), Snail Fever, Soft chancre, Southern tick associated rash illness, Sparganosis, Spelunker's disease, Sporadic typhus, Sporotrichosis, Spotted fever, Spring, St. Louis encephalitis, Staphylococcal Food Poisoning, Staphylococcal Infection, Strep. throat, Streptococcal Disease, Streptococcal Toxic-Shock Syndrome, Strongyloiciasis, Stye, Subacute Sclerosing Panencephalitis, Subacute Sclerosing Panencephalitis (SSPE), Sudden Acute Respiratory Syndrome, Sudden Rash, Swimmer's ear, Swimmer's Itch, Swimming Pool conjunctivitis, Sylvatic yellow fever, Syphilis, Systemic Inflammatory Response Syndrome (SIRS), Tabes dorsalis (tertiary syphilis), Taeniasis, Taiga encephalitis, Tanner's disease, Tapeworm infections, Temporal lobe encephalitis, Temporal lobe encephalitis, tetani (Lock Jaw), Tetanus Infection, Threadworm infections, Thrush, Tick, Tick typhus, Tinea barbae, Tinea capitis, Tinea corporis, Tinea cruris, Tinea manuum, Tinea nigra, Tinea pedis, Tinea unguium, Tinea versicolor, Torulopsosis, Torulosis, Toxic Shock Syndrome, Toxoplasmosis, transmissible spongioform (CJD), Traveler's diarrhea, Trench fever 5, Trichinellosis, Trichomoniasis, Trichomycosis axillaris, Trichuriasis, Tropical Spastic Paraparesis (TSP), Trypanosomiasis, Tuberculosis (TB), Tuberculousis, Tularemia, Typhoid Fever, Typhus fever, Ulcus nolle, Undulant fever, Urban yellow fever, Urethritis, Vaginitis, Vaginosis, Vancomycin Intermediate (VISA), Vancomycin Resistant (VRSA), Varicella, Venezuelan Equine encephalitis, Verruga peruana, Vibrio cholerae (Cholera), Vibriosis (Vibrio), Vincent's disease or Trench mouth, Viral conjunctivitis, Viral Meningitis, Viral meningoencephalitis, Viral rash, Visceral Larval Migrans, Vomito negro, Vulvovaginitis, Warts, Waterhouse, Weirs disease, West Nile Fever, Western equine encephalitis, Whipple's disease, Whipworm infection, White Piedra, Whitlow, Whitmore's disease, Winter diarrhea, Wolhynia fever, Wool sorters' disease, Yaws, Yellow Fever, Yersinosis, Yersinosis (Yersinia), Zahorsky's disease, Zika virus disease, Zoster, Zygomycosis, John Cunningham Virus (JCV), Human immunodeficiency virus (HIV), Influenza virus, Hepatitis B, Hepatitis C, Hepatitis D, Respiratory syncytial virus (RSV), Herpes simplex virus 1 and 2, Human Cytomegalovirus, Epstein-Barr virus, Varicella zoster virus, Coronaviruses Poxviruses, Enterovirus 71, Rubella virus, Human papilloma virus, Streptococcus pneumoniae, Streptococcus viridans Staphylococcus aureus (S. aureus), Methicillin-resistant Staphylococcus aureus (MRSA), Vancomycin-intermediate Staphylococcus aureus (VISA), Vancomycin resistant Staphylococcus aureus (VRSA), Staphylococcus epidermidis (S. epidermidis), Clostridium Tetani, Bordetella pertussis, Bordetella paratussis, Mycobacterium, Francisella tularensis, Toxoplasma gondii, Candida (C. albicans, C. glabrata, C. parapsilosis, C. tropicalis, C. krusei and C. lusitaniae) and/or any other infectious diseases, disorders or syndromes.

Various toxins may be treated with the pharmaceutical compositions, viral particles, of the present disclosure. Non-limited examples of toxins include Ricin, Bacillus anthracis, Shiga toxin and Shiga-like toxin, Botulinum toxins.

Various tropical diseases may be treated with pharmaceutical compositions, viral particles, of the present disclosure. Non-limited examples of tropical diseases include Chikungunya fever, Dengue fever, Chagas disease, Rabies, Malaria, Ebola virus, Marburg virus, West Nile Virus, Yellow Fever, Japanese encephalitis virus, St. Louis encephalitis virus.

Various foodborne illnesses and gastroenteritis may be treated with pharmaceutical compositions, viral particles, of the present disclosure. Non-limited examples of foodborne illnesses and gastroenteritis include Rotavirus, Norwalk virus (Norovirus), Campylobacter jejuni, Clostridium difficile, Entamoeba histolytica, Helicobacter pyroli, Enterotoxin B of Staphylococcus aureus, Hepatitis A virus (HAV), Hepatitis E, Listeria monocytogenes, Salmonella, Clostridium perfringens, and Salmonella.

Various infectious agents may be treated with pharmaceutical compositions, viral particles, of the present disclosure. Non-limited examples of infectious agents include adenoviruses, Anaplasma phagocytophilium, Ascaris lumbricoides, Bacillus anthracis, Bacillus cereus, Bacteroides sp, Barmah Forest virus, Bartonella bacilliformis, Bartonella henselae, Bartonella quintana, beta-toxin of Clostridium perfringens, Bordetella pertussis, Bordetella parapertussis, Borrelia burgdorferi, Borrelia tutyctutotol, Borrelia recurrentis, Borrelia sp., Botulinum toxin, Brucella sp., Burkholderia pseudomallei, California encephalitis virus, Campylobacter, Candida albicans, chikungunya virus, Chlamydia psittaci, Chlamydia trachomatis, Clonorchis sinensis, Clostridium difficile bacteria, Clostridium tetani, Colorado tick fever virus, Corynebacterium diphtheriae, Corynebacterium minutissimum, Coxiella burnetii, coxsackie A, coxsackie B, Crimean-Congo hemorrhagic fever virus, cytomegalovirus, dengue virus, Eastern Equine encephalitis virus, Ebola viruses, echovirus, Ehrlichia chaffeensis., Ehrlichia equi., Ehrlichia sp., Entamoeba histolytica, Enterobacter.sp Enterococcus feacalis, Enterovirus 71, Epstein-Barr virus (EBV), Erysipelothrix rhusiopathiae, Escherichia Flavivirus, Fusobacterium necrophorum, Gardnerella vaginalis, Group B streptococcus, Haemophilus aegyptius, Haemophilus thicreyi, Haemophilus infitienzae, hantavirus, Helicobacter pylori, Hepatitis A, Hepatitis B, Hepatitis C, Hepatitis D, Hepatitis E, herpes simplex virus 1 and 2, human herpes virus 6, human herpes Virus 8, human immunodeficiency. virus 1 and 2, human T-cell leukemia viruses I and II, influenza viruses (A, B, C), Jamestown Canyon virus, Japanese encephalitis antigenic, Japanese encephalitis virus, John Cunningham virus, juninvirus, Kaposi's Sarcoma-associated Herpes Virus OK SEW), Klebsiella granulomatis, Klebsiella sp., Kyasanur Forest Disease virus, La Crosse virus, Lassavirus, Legionella pneumophila, Leptospira interrogans, Listeria monocytogenes, lymphocytic choriomeningitis virus, lyssavirus, Machupovirus, Marburg virus, measles virus, MFRS coronavirus (MFRS-CoV), Micrococcus sedentarius, Mobiluncus sp., Molluscipoxvirus, Moraxella catarrhalis, Rubeola virus, Mumpsvirus, Mycobacterium leprae, Mycobacterium tuberculosis. Mycobacterium ulcerans, Mycoplasma genitalium, Mycoplasma sp, Nairovirus, Neisseria gonorrhoeae, Neisseria meningitidis, Nocardia, Norwalk virus, norovirus, Omsk hemorrhagic fever virus, papilloma virus, parainfluenza viruses 1-3, parapoxvirus, parvovirus B19, Peptostreptococccus sp., Plasmodium sp., polioviruses types 1, 0.11, and HI, Proteus sp., Pseudomonas aeruginosa, Pseudomonas pseudomallei, Pseudomonas sp., rabies virus, respiratory syncytial virus, ricin toxin, Rickettsia australis, Rickettsia conori, Rickettsia honei, Rickettsia prowazekii, Ross River Virus, rotavirus, rubellavirus, Saint Louis encephalitis, Salmonella Typhi, Sarcoptes scabiei, SARS-associated coronavirus (SARS-CoV), Serratia sp., Shiga toxin and Shiga-like toxin, Shigella sp., Sin Nombre Virus, Snowshoe hare virus, Staphylococcus aureus, Staphylococcus epidermidis, Streptobacillus moniliformis, Streptoccoccus pneumoniae, Streptococcus agalactiae, Streptococcus agalactiae, Streptococcus group A-H, Streptococcus pneumoniae, Streptococcus pyogenes, Treponema pallidum subsp. Pallidum, Treponema pallidum var. carateum, Treponema pallidum var. endemicum, Tropheryma Ureaplasma urealyticum, Varicella-Zoster virus, variola virus, Vibrio cholerae, West Nile virus, yellow fever virus, Yersinia enterocolitica, Yersinia pestis, and Zika virus.

Various rare diseases may be treated with pharmaceutical compositions, viral particles, of the present disclosure. As used herein, the term “rare disease” refers to any disease that affects a small percentage of the population. As a non-limiting example, the rare disease may be Acrocephalosyndactylia, Acrodermatitis, Addison Disease, Adie Syndrome, Alagille Syndrome, Amylose, Amyotrophic Lateral Sclerosis, Angelman Syndrome, Angiolymphoid Hyperplasia with Eosinophilia, Arnold-Chiari Malformation, Arthritis, Juvenile Rheumatoid, Asperger Syndrome, Bardet-Biedl Syndrome, Barrett Esophagus, Beckwith-Wiedemann Syndrome, Behcet Syndrome, Bloom Syndrome, Bowen's Disease, Brachial Plexus Neuropathies, Brown-Sequard Syndrome, Budd-Chiari Syndrome, Burkitt Lymphoma, Carcinoma 256, Walker, Caroli Disease, Charcot-Marie-Tooth Disease, Chediak-Higashi Syndrome, Chiari-Frommel Syndrome, Chondrodysplasia Punctata, Colonic Pseudo-Obstruction, Colorectal Neoplasms, Hereditary Nonpolyposis, Craniofacial Dysostosis, Creutzfeldt-Jakob Syndrome, Crohn Disease, Cushing Syndrome, Cystic Fibrosis, Dandy-Walker Syndrome, De Lange Syndrome, Dementia, Vascular, Dermatitis Herpetiformis, DiGeorge Syndrome, Diffuse Cerebral Sclerosis of Schilder, Duane Retraction Syndrome, Dupuytren Contracture, Ebstein Anomaly, Eisenmenger Complex, Ellis-Van Creveld Syndrome, Encephalitis, Enchondromatosis, Epidermal Necrolysis, Toxic, Facial Hemiatrophy, Factor XII Deficiency, Fanconi Anemia, Felty's Syndrome, Fibrous Dysplasia, Polyostotic, Fox-Fordyce Disease, Friedreich Ataxia, Fusobacterium, Gardner Syndrome, Gaucher Disease, Gerstmann Syndrome, Giant Lymph Node Hyperplasia, Glycogen Storage Disease Type I, Glycogen Storage Disease Type II, Glycogen Storage Disease Type IV, Glycogen Storage Disease Type V, Glycogen Storage Disease Type VII, Goldenhar Syndrome, Guillain-Barre Syndrome, Hallermann's Syndrome, Hamartoma Syndrome, Multiple, Hartnup Disease, Hepatolenticular Degeneration, Hepatolenticular Degeneration, Hereditary Sensory and Motor Neuropathy, Hirschsprung Disease, Histiocytic Necrotizing Lymphadenitis, Histiocytosis, Langerhans-Cell, Hodgkin Disease, Horner Syndrome, Huntington Disease, Hyperaldosteronism, Hyperhidrosis, Hyperostosis, Diffuse Idiopathic Skeletal, Hypopituitarism, Inappropriate ADH Syndrome, Intestinal Polyps, Isaacs Syndrome, Kartagener Syndrome, Kearns-Sayre Syndrome, Klippel-Feil Syndrome, Klippel-Trenaunay-Weber Syndrome, Kluver-Bucy Syndrome, Korsakoff Syndrome, Lafora Disease, Lambert-Eaton Myasthenic Syndrome, Landau Kleffner Syndrome, Langer-Giedion Syndrome, Leigh Disease, Lesch-Nyhan Syndrome, Leukodystrophy, Globoid Cell, Li-Fraumeni Syndrome, Long Q T Syndrome, Machado-Joseph Disease, Mallory-Weiss Syndrome, Marek Disease, Marfan Syndrome, Meckel Diverticulum, Meige Syndrome, Melkersson-Rosenthal Syndrome, Meniere Disease, Mikulicz' Disease, Miller Fisher Syndrome, Mobius Syndrome, Moyamoya Disease, Mucocutaneous Lymph Node Syndrome, Mucopolysaccharidosis I, Mucopolysaccharidosis II, Mucopolysaccharidosis Mucopolysaccharidosis IV, Mucopolysaccharidosis VI, Multiple Endocrine Neoplasia Type 1, Munchausen Syndrome by Proxy, Muscular Atrophy, Spinal, Narcolepsy, Neuroaxonal Dystrophies, Neuromyelitis Optica, Neuronal Ceroid-Lipofuscinoses, Niemann-Pick Diseases, Noonan Syndrome, Optic Atrophies, Hereditary, Osteitis Deformans, Osteochondritis, Osteochondrodysplasias, Osteolysis, Essential, Paget Disease Extramammary, Paget's Disease, Mammary, Panniculitis, Nodular Nonsuppurative, Papillon-Lefevre Disease, Paralysis, Pelizaeus-Merzbacher Disease, Pemphigus, Benign Familial, Penile Induration, Pericarditis, Constrictive, Peroxisomal Disorders, Peutz-Jeghers Syndrome, Pick Disease of the Brain, Pierre Robin Syndrome, Pigmentation Disorders, Pityriasis Lichenoides, Polycystic Ovary Syndrome, Polyendocrinopathies, Autoimmune, Prader-Willi Syndrome, Pupil Disorders, Rett Syndrome, Reye Syndrome, Rubinstein-Taybi Syndrome, Sandhoff Disease, Sarcoma, Ewing's, Schnitzler Syndrome, Sjogren's Syndrome, Sjogren-Larsson Syndrome, Smith-Lemli-Opitz Syndrome, Spinal Muscular Atrophies of Childhood, Sturge-Weber Syndrome, Sweating, Gustatory, Takayasu Arteritis, Tangier Disease, Tay-Sachs Disease, Thromboangiitis Obliterans, Thyroiditis, Autoimmune, Tietze's Syndrome, Togaviridae Infections, Tolosa-Ilunt Syndrome, Tourette Syndrome, Uveomeningoencephalitic Syndrome, Waardenburg's Syndrome, Wegener Granulomatosis, Weil Disease, Werner Syndrome, Williams Syndrome, Wilms Tumor, Wolff-Parkinson-White Syndrome, Wolfram Syndrome, Wolman Disease, Zellweger Syndrome, Zollinger-Ellison Syndrome, and von Willebrand Diseases.

Various autoimmune diseases and autoimmune-related diseases may be treated with pharmaceutical compositions, viral particles, of the present disclosure. As used herein, the term “autoimmune disease” refers to a disease in which the body produces antibodies that attack its own tissues. As a non-limiting example, the autoimmune disease may be Acute Disseminated Encephalomyelitis (ADEM), Acute necrotizing hemorrhagic leukoencephalitis, Addison's, disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal & neuronal neuropathies, Mc) disease, Behcet's disease, Bullous pemphigoid, Cardiomyopathy, Castleman disease, Celiac disease, Chagas disease, Chronic fatigue syndrome**, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia**, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Ulcerative colitis, Undifferentiated connective tissue disease (UCTD), Uveitis, Vasculitis, Vesiculobullous dermatosis, Vitiligo, and Wegener's granulomatosis (now termed Granulomatosis with Polyangiitis (GPA).

Various kidney diseases may be treated with pharmaceutical compositions, viral particles, of the present disclosure. As a non-limiting example, the kidney disease Abderhal den-Kaufmann-Lignac syndrome (Nephropathic Cystinosis), Abdominal Compartment Syndrome, Acute Kidney Failure/Acute Kidney Injury, Acute Lobar Nephronia, Acute Phosphate Nephropathy, Acute Tubular Necrosis, Adenine Phosphofibosyltransferase Deficiency, Adenovirus Nephritis, Alport Syndrome, Amyloidosis, ANCA Vasculitis Related to Endocarditis and Other Infections, Angiomyolipoma, Analgesic Nephropathy, Anorexia Nervosa and Kidney Disease, Angiotensin Antibodies and Focal Segmental Glomerulosclerosis, Antiphospholipid Syndrome, Anti-TNF-α. Therapy-related Glomerulonephritis, APOL1 Mutations, Apparent Mineralocorticoid Excess Syndrome, Aristolochic Acid Nephropathy, Chinese Herbal Nephropathy, Balkan Endemic Nephropathy, Bartter Syndrome, Beeturia, 13-Thalassemia Renal Disease, Bile Cast Nephropathy, BK Polyoma Virus Nephropathy in the Native Kidney, Bladder Rupture, Bladder Sphincter Dyssynergia, Bladder Tamponade, Border-Crossers' Nephropathy, Bourbon Virus and Acute Kidney Injury, Burnt Sugarcane Harvesting and Acute Renal Dysfunction, Byetta and Renal Failure, Clq Nephropathy, Cannabinoid Hyperemesis Acute Renal Failure, Cardiorenal syndrome, Carfilzomib-Induced Renal Injury, CFHR5 nephropathy, Charcot-Marie-Tooth Disease with Glomerulopathy, Cherry Concentrate and Acute Kidney Injury, Cholesterol Emboli, Churg-Strauss syndrome, Chyluria, Colistin Nephrotoxicity, Collagenofibrotic Glomerulopathy, Collapsing Glomerulopathy, Collapsing Glomerulopathy Related to CMV, Congenital Nephrotic Syndrome, Conorenal syndrome (Mainzer-Saldino Syndrome or Saldino-Mainzer Disease), Contrast Nephropathy, Copper Sulpfate Intoxication, Cortical Necrosis, Crizotinib-related Acute Kidney Injury, Cryoglobuinemia, Crystalglobulin-Induced Nephropathy, Crystal-Induced Acute Kidney injury, Cystic Kidney Disease, Acquired, Cystinuria, Dasatinib-Induced Nephrotic-Range Proteinuria, Dense Deposit Disease (MPGN Type 2), Dent Disease (X-linked Recessive Nephrolithiasis), Dialysis Disequilibrium Syndrome, Diabetes and Diabetic Kidney Disease, Diabetes insipidus, Dietary Supplements and Renal Failure, Drugs of Abuse and Kidney Disease, Duplicated Ureter, EAST syndrome, Ebola and the Kidney, Ectopic Kidney, Ectopic Ureter, Edema, Swelling, Erdheim-Chester Disease, Fabry's Disease, Familial Hypocalciuric Hypercalcemia, Fanconi Syndrome, Fraser syndrome, Fibronectin Glomerulopathy, Fibrillary Glomerulonephritis and Iminunotactoid Glomerulopathy, Fraley syndrome, Focal Segmental Glomerulosclerosis, Focal Sclerosis, Focal Glomerulosclerosis, Galloway Mowat syndrome, Giant Cell (Temporal) Arteritis with Kidney Involvement, Gestational Hypertension, Gitelman Syndrome, Glomerular Diseases, Glomerular Tubular Reflux, Glycosuria, Goodpasture Syndrome, Hair Dye ingestion and Acute Kidney Injury, Hantavirus Infection Podocytopathy, Hematuria (Blood in Urine), Hemolytic Uremic Syndrome (HUS), Atypical Hemolytic Uremic Syndrome (aHUS), Hemophagocytic Syndrome, Hemorrhagic Cystitis, Hemorrhagic Fever with Renal Syndrome (HFRS, Hantavirus Renal Disease, Korean Hemorrhagic Fever, Epidemic Hemorrhagic Fever, Nephropathis Epidemica), Hemosiderosis related to Paroxysmal Nocturnal Hemoglobinuria and Hemolytic Anemia, Hepatic Glomerulopathy, Hepatic Veno-Occlusive Disease, Sinusoidal Obstruction Syndrome, Hepatitis C-Associated Renal Disease, Hepatorenal Syndrome, Herbal Supplements and Kidney Disease, High Blood Pressure and Kidney Disease, HIV-Associated Nephropathy (HIVAN), Horseshoe Kidney (Renal Fusion), Hunner's Ulcer, Hyperaldosteronism, Hypercalcemia, Hyperkalemia, Hypermagnesemia, Hypernatremia, Hyperoxaluria, Hyperphosphatemia, Hypocalcemia, Hypokalemia, Hypokalemia-induced renal dysfunction, Hypokalemic Periodic Paralysis, Hypomagnesemia, Hyponatremia, Hypophosphatemia, IgA Nephropathy, IgG4 Nephropathy, Interstitial Cystitis, Painful Bladder Syndrome (Questionnaire), Interstitial Nephritis, Ivemark's syndrome, Ketamine-Associated Bladder Dysfunction, Kidney Stones, Nephrolithiasis, Kombucha Tea Toxicity, Lead Nephropathy and Lead-Related Nephrotoxicity, Leptospirosis Renal Disease, Light Chain Deposition Disease, Monoclonal Immunoglobulin Deposition Disease, Liddle Syndrome, Lightwood-Albright Syndrome, Lipoprotein Glomerulopathy, Lithium Nephrotoxicity, LMX1B Mutations Cause Hereditary FSGS, Loin Pain Hematuria, Lupus, Systemic Lupus Erythematosis, Lupus Kidney Disease, Lupus Nephritis, Lupus Nephritis with Antineutrophil Cytoplasmic Antibody Seropositivity, Lyme Disease-Associated Glomerulonephritis, Malarial Nephropathy, Malignancy-Associated Renal Disease, Malignant Hypertension, Malakoplakia, Meatal Stenosis, Medullary Cystic Kidney Disease, Medullary Sponge Kidney, Megaureter, Melamine Toxicity and the Kidney, Membranoproliferative Glomerulonephritis, Membranous Nephropathy, MesoAmerican Nephropathy, Metabolic Acidosis, Metabolic Alkalosis, Methotrexate-related Renal Failure, Microscopic Polyangiitis, Milk-alkalai syndrome, Minimal Change Disease, MDMA (Molly; Ecstacy; 3,4-Methylenedioxymethamphetamine) and Kidney Failure, Multicystic dysplastic kidney, Multiple Myeloma, Myeloproliferative Neoplasms and Glomerulopathy, Nail-patella Syndrome, Nephrocalcinosis, Nephrogenic Systemic Fibrosis, Nephroptosis (Floating Kidney, Renal Ptosis), Nephrotic Syndrome, Neurogenic Bladder, Nodular Glomerulosclerosis, Non-Gonococcal Urethritis, Nutcracker syndrome, Orofaciodigital Syndrome, Orotic Aciduria, Orthostatic Hypotension, Orthostatic Proteinuria, Osmotic Diuresis, Ovarian Hyperstimulation Syndrome, Page Kidney, Papillary Necrosis, Papillorenal Syndrome (Renal-Coloboma Syndrome, Isolated Renal Hypoplasia), Parvovirus B19 and the Kidney. The Peritoneal-Renal Syndrome, Posterior Urethral Valve, Post-infectious Glomerulonephritis, Post-streptococcal Glomerulonephritis, Polyarteritis Nodosa, Polycystic Kidney Disease, Posterior Urethral Valves, Preeclampsia, Propofol infusion syndrome, Proliferative Glomerulonephritis with Monoclonal IgG Deposits (Nasr Disease), Propolis (Honeybee Resin) Related Renal Failure, Proteinuria (Protein in Urine), Pseudohyperaldosteronism, Pseudohypobicarbonatemia, Pseudohypoparathyroidism, Pulmonary-Renal Syndrome, Pyelonephritis (Kidney Infection), Pyonephrosis, Radiation Nephropathy, Ranolazine and the Kidney, Refeeding syndrome, Reflux Nephropathy, Rapidly Progressive Glomerulonephritis, Renal Abscess, Peripnephric Abscess, Renal Agenesis, Renal Arcuate Vein Microthrombi-Associated Acute Kidney Injury, Renal Artery Aneurysm, Renal Artery Stenosis, Renal Cell Cancer, Renal Cyst, Renal Hypouricemia with Exercise-induced Acute Renal Failure, Renal Infarction, Renal Osteodystrophy, Renal Tubular Acidosis, Renin Secreting Tumors (Juxtaglomerular Cell Tumor), Reset Osmostat, Retrocaval Ureter, Retroperitoneal Fibrosis, Rhabdomyolysis, Rhabdomyolysis related to Bariatric Sugery, Rheumatoid Arthritis-Associated Renal Disease, Sarcoidosis Renal Disease, Salt Wasting, Renal and Cerebral, Schistosomiasis and Glomerular Disease, Schimke immuno-osseous dysplasia, Scleroderma Renal Crisis, Serpentine Fibula-Polycystic Kidney Syndrome, Exner Syndrome, Sickle Cell Nephropathy, Silica Exposure and Chronic Kidney Disease, Sri Lankan Farmers' Kidney Disease, Sjögren's Syndrome and Renal Disease, Synthetic Cannabinoid Use and Acute Kidney Injury, Kidney Disease Following Hematopoietic Cell Transplantation, Kidney Disease Related to Stem Cell Transplantation, Thin Basement Membrane Disease, Benign Familial Hematuria, Trigonitis, Tuberculosis, Genitourinary, Tuberous Sclerosis, Tubular Dysgenesis, Immune Complex Tubulointerstitial Nephritis Due to Autoantibodies to the Proximal Tubule Brush Border, Tumor Lysis Syndrome, Uremia, Uremic Optic Neuropathy, Ureteritis Cystica, Ureterocele, Urethral Caruncle, Urethral Stricture, Urinary incontinence, Urinary Tract Infection, Urinary Tract Obstruction, Vesicointestinal Fistula, Vesicoureteral Reflux, Volatile Anesthetics and Acute Kidney Injury, Von Hippel-Lindau Disease, Waldenstrom's Macroglobulinemic Glomerulonephritis, Warfarin-Related Nephropathy, Wasp Stings and Acute Kidney Injury, Wegener's Granulomatosis, Granulomatosis with Polyangiitis, West Nile Virus and Chronic Kidney Disease, and Wunderlich syndrome.

Various cardiovascular diseases may be treated with pharmaceutical compositions, viral particles, of the present disclosure. As a non-limiting example, the cardiovascular disease may be Ischemic heart disease also known as coronary artery disease, cerebrovascular disease (Stroke), Peripheral vascular disease, Heart failure, Rheumatic heart disease, and Congenital heart disease.

Various antibody deficiencies may be treated with pharmaceutical compositions, viral particles, of the present disclosure. As a non-limiting example, the antibody deficiencies may be X-Linked Agammaglobulinemia (XLA), Autosomal Recessive Agammaglobulinemia (ARA), Common Variable Immune Deficiency (LVID), IgG (IgGE IgG2, IgG3 and IgG4) Subclass Deficiency, Selective IgA Deficiency, Specific Antibody Deficiency (SAD), Transient Hypogammaglobulinemia of Infancy, Antibody Deficiency with Normal or Elevated Immunoglobulins, Selective IgM Deficiency, Immunodeficiency with Thymoma (Good's Syndrome), Transcobalamin II Deficiency, Warts, Hypogammaglobulinemia, Infection, Myelokathexis (WHIM) Syndrome, Drug-Induced Antibody Deficiency, Kappa Chain Deficiency, Heavy Chain Deficiencies, Post-Meiotic Segregation (PMS2) Disorder, and Unspecified Hypogammaglobulinemia.

Various ocular diseases may be treated with pharmaceutical compositions, viral particles, of the present disclosure. As a non-limiting example, the ocular disease may be thyroid eye disease (TED), Graves' disease (GD) and orbitopathy, Retina Degeneration, Cataract, optic atrophy, macular degeneration, Leber congenital amaurosis, retinal degeneration, cone-rod dystrophy, Usher syndrome, leopard syndrome, photophobia, and photoaversion.

Various neurological diseases may be treated with pharmaceutical compositions, viral particles, of the present disclosure. As a non-limiting example, the neurological disease may be Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain injury, Brown-Sequard Syndrome, Bulbospinal Muscular Atrophy, Cerebral Autosomal Dominant Arteriopathy with Sub-cortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt Jakob Disease, Cumulative Trauma Disorders, Cushing's Syndrome, Cytomegalic Inclusion Body Disease, Cytomegalovirus Infection, Dancing-Eyes-Dancing Feet Syndrome, Dandy-Walker Syndrome, Dawson Disease, De Morsier's Syndrome, Dejerine-Klumpke Palsy, Dementia, Dementia-Multi-Infarct Dementia-Semantic, Dementia-Subcortical, Dementia With Lewy Bodies, Dentate Cerebellar Ataxia, Dentatorubral Atrophy, Dertnatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyspraxia, Dyssynergia Cerebeillaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Efemiplegia. Alterans, Hereditary Neuropathies, Hereditary Spastic Paraplegia, fIeredopathia Atactica Pollyneuritiformis, Herpes Zoster, Herpes Zoster Oticus, Birayam a Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus-Normal Pressure, Hydromyelia, Hypercortisolism, Hypersomnia, Hypertonia, Hypotonia, Hypoxia, Immunes Mediated Encephalomyelitis, Inclusion Body Myositis, incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kitiver-Bucy Syndrome, Korsakoffs Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus-Neurological Sequelae, Lyme Disease-Neurological Complications, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia m Congenital, Myasthenia Gravis, Myelinoclastic Diffuse Sclerosis, Myoclonic Encephalopathy of Infants, Myoclonus, Myopathy, Myopathy-Congenital, Myopathy-Thyrotoxic, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy-Hereditary, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain-Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease-Infantile, Repetitive Motion Disorders, Repetitive Stress injuries, Restless Legs Syndrome, Retrovirus-Associated Myelopathy, Rett Syndrome, Reyes Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Von Economo's Disease, Von Hippel-Lindau Disease (VHL), Von. Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, IX-Linked Spinal and Bulbar Muscular Atrophy.

Various psychological disorders may be treated with pharmaceutical compositions, viral particles, of the present disclosure. As a non-limiting example, the psychological disorders may be Aboulia, Absence epilepsy, Acute stress Disorder, Adjustment Disorders, Adverse effects of medication NOS, Age related cognitive decline, Agoraphobia, Alcohol Addiction, Alzheimer's Disease, Amnesia (also known as Amnestic Disorder), Amphetamine Addiction, Anorexia Nervosa, Anterograde amnesia, Antisocial personality disorder (also known as Sociopathy), Anxiety Disorder (Also known as Generalized Anxiety Disorder), Anxiolytic related disorders, Asperger's Syndrome (now part of Autism Spectrum Disorder), Attention Deficit Disorder (Also known as ADD), Attention Deficit Hyperactivity Disorder (Also known as ADM), Autism Spectrum Disorder (also known as Autism), Autophagia, Avoidant Personality Disorder, Barbiturate related disorders, Benzodiazepine related disorders, Bereavement, Bibliomania, Binge Eating Disorder, Bipolar disorder (also known as Manic Depression, includes Bipolar I and Bipolar II), Body Dysmorphic Disorder, Borderline intellectual functioning, Borderline Personality Disorder, Breathing-Related Sleep Disorder, Brief Psychotic Disorder, Bruxism, Bulimia Nervosa, Caffeine Addiction, Cannabis Addiction, Catatonic disorder, Catatonic schizophrenia, Childhood amnesia, Childhood Disintegrative Disorder (now part of Autism Spectrum Disorder), Childhood Onset Fluency Disorder (formerly known as Stuttering), Circadian Rhythm Disorders, Claustrophobia, Cocaine related disorders, Communication disorder, Conduct Disorder, Conversion Disorder, Cotard delusion, Cyclothymia (also known as Cyclothymic Disorder), Delerium, Delusional Disorder, dementia, Dependent Personality Disorder (also known as Asthenic Personality Disorder), Depersonalization disorder (now known as Depersonalization/Derealization Disorder), Depression (also known as Major Depressive Disorder), Depressive personality disorder, Derealization disorder (now known as Depersonalization/Derealization Disorder), Dermotillomani a, Desynchronosis, Developmental coordination disorder, Diogenes Syndrome, Disorder of written expression, Dispareunia, Dissocial Personality Disorder, Dissociative Amnesia, Dissociative Fugue, Dissociative Identity Disorder (formerly known as Multiple Personality Disorder), Down syndrome, Dyslexia, Dyspareunia, Dysthymia (now known as Persistent Depressive Disorder), Eating disorder NOS, Ekbom's Syndrome (Delusional Parasitosis), Emotionally unstable personality disorder, Encopresis, Enuresis (bedwetting), Erotomania, Exhibitionistic Disorder, Expressive language disorder, Factitious Disorder, Female Sexual Disorders, Fetishistic Disorder, Folie à deux, Fregoli delusion, Frotteuristic Disorder, Fugue State, Ganser syndrome, Gambling Addiction, Gender Dysphoria (formerly known as Gender Identity Disorder), Generalized Anxiety Disorder, General adaptation syndrome, Grandiose delusions, Hallucinogen Addiction, Haitlose personality disorder, Histrionic Personality Disorder, Primary hypersomnia, Huntington's Disease, Hypoactive sexual desire disorder, Hypochondriasis, Hypomania, Hyperkinetic syndrome, Hypersomnia, Hysteria, Impulse control disorder, Impulse control disorder NOS, Inhalant Addiction, Insomnia, Intellectual Development Disorder, Intermittent Explosive Disorder, Joubert syndrome, Kleptomania, Korsakoff's syndrome, Lacunar amnesia, Language Disorder, Learning Disorders, Major Depression (also known as Major Depressive Disorder), major depressive disorder, Male Sexual Disorders, Malingering, Mathematics disorder, Medication-related disorder, Melancholia, Mental Retardation (now known as Intellectual Development Disorder), Misophonia, Morbid jealousy, Multiple Personality Disorder (now known as Dissociative Identity Disorder), Munchausen Syndrome, Munchausen by Proxy, Narcissistic Personality Disorder, Narcolepsy, Neglect of child, Neurocognitive Disorder (formerly known as Dementia), Neuroleptic-related disorder, Nightmare Disorder, Non Rapid Eye Movement, Obsessive-Compulsive Disorder, Obsessive-Compulsive Personality Disorder (also known as Anankastic Personality Disorder), Oneirophrenia, Onychophagia, Opioid Addiction, Oppositional Defiant Disorder, Orthorexia (ON), Pain disorder, Panic attacks, Panic Disorder, Paranoid Personality Disorder, Parkinson's Disease, Partner relational problem, Passive-aggressive personality disorder, Pathological gambling, Pedophilic Disorder, Perfectionism, Persecutory delusion, Persistent Depressive Disorder (also known as Dysthymia), Personality change due to a general medical condition, Personality disorder, Pervasive developmental disorder (PDD), Phencyclidine related disorder, Phobic disorder, Phonological disorder, Physical abuse, Pica, Polysubstance related disorder, Postpartum Depression, Post-traumatic embitterment disorder (PTSD), Post Traumatic Stress Disorder, Premature ejaculation, Premenstrual Dysphoric Disorder, Psychogenic amnesia, Psychological factor affecting medical condition, Psychoneurotic personality disorder, Psychotic disorder, not otherwise specified, Pyromania, Reactive Attachment Disorder, Reading disorder, Recurrent brief depression, Relational disorder, REM Sleep Behavior Disorder, Restless Leg Syndrome, Retrograde amnesia, Retts Disorder (now part of Autism Spectrum Disorder), Rumination syndrome, Sadistic personality disorder, Schizoaffective Disorder, Schizoid Personality Disorder, Schizophrenia, Schizophreniform disorder, Schizotypal Personality Disorder, Seasonal Affective Disorder, Sedative, Hypnotic, or Anxiolytic Addiction, Selective Mutism, Self-defeating personality disorder, Separation Anxiety Disorder, Sexual Disorders Female, Sexual Disorders Male, Sexual Addiction, Sexual Masochism Disorder, Sexual Sadism Disorder, Shared Psychotic Disorder, Sleep Arousal Disorders, Sleep Paralysis, Sleep Terror Disorder (now part of Nightmare Disorder, Social Anxiety Disorder, Somatization Disorder, Specific Phobias, Stendhal syndrome, Stereotypic movement disorder, Stimulant Addiction, Stuttering (now known as Childhood Onset Fluency Disorder), Substance related disorder, Tardive dyskinesia, Tobacco Addiction, Tourettes Syndrome, Transient tic disorder, Transient global amnesia, Transvestic Disorder, Trichotillomania, Undifferentiated Somatoform Disorder, Vaginismus, and Voyeuristic Disorder.

Various lung diseases may be treated with pharmaceutical compositions, viral particles, of the present disclosure. As a non-limiting example, the lung diseases may be Asbestosis, Asthma, Bronchiectasis, Bronchitis, Chronic Cough, Chronic Obstructive Pulmonary Disease (COPT)), Croup, Cystic Fibrosis, Hantavirus, Idiopathic Pulmonary Fibrosis, Pertussis, Pleurisy, Pneumonia, Pulmonary Embolism, Pulmonary Hypertension, Sarcoidosis, Sleep Apnea, Spirometry, Sudden Infant Death Syndrome (SIDS), Tuberculosis, klagille Syndrome, Autoimmune Hepatitis, Biliary Atresia, Cirrhosis, ERCP (Endoscopic Retrograde Cholangiopancreatography), and Hemochromatosis. Nonalcoholic Steatohepatitis, Porphyria, Primary Biliary Cirrhosis, Primary Sclerosing Cholangitis.

Various bone diseases may be treated with pharmaceutical compositions, viral particles, of the present disclosure. As a non-limiting example, the bone diseases may be osteoporosis, neurofibromatosis, osteogenesis imperfecta (OI), rickets, osteosarcoma, achondroplasia, fracture, osteomyelitis, Ewing tumour of bone, osteomalacia, hip dysplasia, Paget disease of bone, marble bone disease, osteochondroma, bone cancer, bone disease, osteochondrosis, osteoma, fibrous dysplasia, cleidocranial dysostosis, osteoclastoma, bone cyst, metabolic bone disease, melorheostosis, callus, Caffey syndrome, and mandibulofacial dysostosis.

Various blood diseases may be treated with pharmaceutical compositions, viral particles, of the present disclosure. As a non-limiting example, the blood diseases may be Anemia and CKT) (for health care professionals), Aplastic Anemia and Myelodysplastic Syndromes, Deep Vein Thrombosis, Hemochromatosis, Hemophilia, Henoch-Schonlein Purpura, Idiopathic Thrombocytopenic Purpura, Tron-Defi ciency Anemia, Pernicious Anemia, Pulmonary Embolism, Sickle Cell Anemia, Sickle Cell Trait and Other Hemoglobinopathies, Thalassemia, Thrombotic Thrombocytopenic Purpura, and Von Willebrand Disease.

Various diseases associated with TNF alpha may be treated with the pharmaceutical compositions, viral particles, of the present disclosure. As a non-limiting example, the disease may be respiratory disorder; asthma; allergic and nonallergic asthma; asthma due to infection; asthma due to infection with respiratory syncytial virus (RSV); chronic obstructive pulmonary disease (COPD); a condition involving airway inflammation; eosinophilia; fibrosis and excess mucus production; cystic fibrosis; pulmonary fibrosis; an atopic disorder; atopic dermatitis; urticaria; eczema; allergic rhinitis; allergic enterogastritis; an inflammatory and/or autoimmune condition of the skin; an inflammatory and/or autoimmune condition of gastrointestinal organs; inflammatory bowel diseases (IBD); ulcerative colitis; Crohn's disease; an inflammatory and/or autoimmune condition of the liver; liver cirrhosis; liver fibrosis; liver fibrosis caused by hepatitis B and/or C virus; scleroderma; tumors or cancers; hepatocellular carcinoma; glioblastoma; lymphoma; Hodgkin's lymphoma; a viral infection; a bacterial infection; a parasitic infection; HTLV-1 infection; suppression of expression of protective type 1 immune responses, and suppression of expression of a protective type 1 immune response during vaccination, rheumatoid arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia greata, seronegative arthropathy, arthropathy, Reiter's disease, psoriatic arthropathy, ulcerative colitic arthropathy, enteropathic synovitis, chlamydia, yersinia and salmonella associated arthropathy, spondyloarthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired immunodeficiency Related Diseases, hepatitis B, hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasculitis of the kidneys, Lyme disease, discoid lupus erythematosus, male infertility idiopathic or NOS, spenn autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjorgren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo acute liver disease, chronic liver diseases, alcoholic cirrhosis, alcohol-induced liver injury, choleostasis, idiosyncratic liver disease, drug-Induced hepatitis, non-alcoholic steatohepatitis, allergy and asthma, group B streptococci (GBS) infection, mental disorders (e.g., depression and schizophrenia), Th2 Type and Th1 Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma) abetalipoproteinemia, acrocyanosis, acute and chronic parasitic or infectious processes, acute leukemia, acute lymphoblastic leukemia (ALL), acute myeloid leukemia. (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti-CD3 therapy, antiphospholipid syndrome, anti-receptor hypersensitivity reactions, aortic and peripheral aneurysms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multi focal atrial tachycardia, chemotherapy associated disorders, chronic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, corpulmonale, coronary artery disease, Creutzfeldt-Jakob disease, culture negative sepsis, cystic fibrosis, cytokine therapy associated disorders, dementia pugilistica, demyelinating diseases, dengue hemorrhagic fever, dermatitis, dermatologic conditions, diabetes, diabetes mellitus, diabetic arteriosclerotic disease, Diffuse Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, Epstein-Barr vim s infection, erythromelalgia, extrapyramidal and cerebellar disorders, familial hemophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallervorden-Spatz disease, Hashimoto's thyroiditis, hay fever, heart transplant rejection, hemochromatosis, hemodialysis, hemolytic uremic syndrome/thrombolytic thrombocytopenic purpura, hemorrhage, hepatitis (A), His bundle arrhythmi as, infection/HIV neuropathy, Hodgkin's disease, hyperkinetic movement disorders, hypersensitivity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, asthenia, infantile spinal muscular atrophy, inflammation of the aorta, influenza a, ionizing radiation exposure, iridocyclitis/uveitis/optic neuritis, ischernia-reperfiusion injury, ischemic stroke, juvenile rheumatoid arthritis (JRA), juvenile spinal muscular atrophy, Kaposi's sarcoma, kidney transplant rejection, legionella, leishmaniasis, leprosy, lesions of the corticospinal system, lipedema, liver transplant rejection, lymphedema, malaria, malignant lymphoma, malignant histiocytosis, malignant melanoma, meningitis, meningococcemia, metabolic/idiopathic, migraine headache, mitochondrial multi-system disorder, mixed connective tissue disease, monoclonal gammopathy, multiple myeloma, multiple systems degenerations (Menzel, Dejerine-Thomas, Shy-Drager, and Machado-Joseph), myasthenia gravis, Mycobacterium avium intracellulare, Mycobacterium tuberculosis, myelodysplastic syndrome, myocardial infarction, myocardial ischemic disorders, nasopharyngeal carcinoma, neonatal chronic lung disease, nephritis, nephrosis, neurodegenerative diseases, neurogenic I muscular atrophies, neutropenic fever, non-Hodgkins lymphoma, occlusion of the abdominal aorta and its branches, occlusive arterial disorders, OKT3© therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherosclerotic disease, peripheral vascular disorders, peritonitis, pernicious anemia, Pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, progressive supranuclear palsy, primary pulmonary hypertension, radiation therapy, Raynaud's phenomenon and disease, Raynaud's disease, Refsum's disease, regular narrow QRS tachycardia, renovascular hypertension, reperfusion injury, restrictive cardiomyopathy, sarcomas, scleroderma, senile chorea, senile dementia of Lewy body type, seronegative arthropathies, shock, sickle cell anemia, skin allograft rejection, skin changes syndrome, small bowel transplant rejection, solid tumors, specific arrhythmias, spinal ataxia, spinocerebellar degenerations, streptococcal myositis, structural lesions of the cerebellum, subacute sclerosing panencephalitis, syncope, syphilis of the cardiovascular system, systemic anaphylaxis, systemic inflammatory response syndrome, systemic onset juvenile rheumatoid arthritis, T-cell or FAB AT L, telangiectasia, thromboangitis obliterans, thrombocytopenia, toxicity, transplants, traumalhemorrhage, type IIl hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, viral encephalitis/aseptic meningitis, viral-associated hemophagocytic syndrome, Wernicke-Korsakoff syndrome, Wilson's disease, xenograft rejection of any organ or tissue, acute coronary syndromes, acute idiopathic polyneuritis, acute inflammatory demyelinating polyradiculoneuropathy, acute ischemia, adult Still's disease, alopecia greata, anaphylaxis, anti-phospholipid antibody syndrome, aplastic anemia, arteriosclerosis, atopic eczema, atopic dermatitis, autoimmune dermatitis, autoimmune disorder associated with streptococcus infection, autoimmune enteropathy, autoimmune hearing loss, autoimmune lymphoproliferative syndrome (ALPS), autoimmune myocarditis, autoimmune premature ovarian failure, blepharitis, bronchiectasis, bullous pemphigoid, cardiovascular disease, catastrophic antiphospholipid syndrome, celiac disease, cervical spondylosis, chronic ischemia, cicatricial pemphigoid, clinically isolated syndrome (CIS) with risk for multiple sclerosis, conjunctivitis, childhood onset psychiatric disorder, chronic obstructive pulmonary disease (COPD), dacryocystitis, dermatomyositis, diabetic retinopathy, diabetes mellitus, disk herniation, disk prolapse, drug induced immune hemolytic anemia, endocarditis, endometriosis, endophthalmitis, episcleritis, erythema multifoune, erythema multiforme major, gestational pemphigoid, Guillain-Barre syndrome (GB S), hay fever, Hughes syndrome, idiopathic Parkinson's disease, idiopathic interstitial pneumonia, IgE-mediated allergy, immune hemolytic anemia, inclusion body myositis, infectious ocular inflammatory disease, inflammatory demyelinating disease, inflammatory heart disease, inflammatory kidney disease, IPF′/LIP, iritis, keratitis, keratojunctivitis sicca, Kussmaul disease or Kussmaul-Meier disease, Landry's paralysis, Langerhan's cell histiocytosis, livedo reticularis, macular degeneration, microscopic polyangiitis, morbus bechterev, motor neuron disorders, mucous membrane pemphigoid, multiple organ failure, myasthenia gravis, myelodysplastic syndrome, myocarditis, nerve root disorders, neuropathy, non-A non-B hepatitis, optic neuritis, osteolysis, ovarian cancer, pauciarticular JRA, peripheral artery occlusive disease (PROD), peripheral vascular disease (PVD), peripheral artery disease (PAD), phlebitis, polyarteritis nodosa (or periarteritis nodosa), polychondritis, polymyalgia rheumatica, poliosis, polyarticular polyendocrine deficiency syndrome, polymyositis, polymyalgia rheumatica (PIER), post-pump syndrome, primary Parkinsonism, prostate and rectal cancer and hematopoietic malignancies (leukemia and lymphoma), prostatitis, pure red cell aplasia, primary adrenal insufficiency, recurrent neuromyelitis optica, restenosis, rheumatic heart disease, sapho (synovitis, acne, pustulosis, hyperostosis, and osteitis), scleroderma, secondary amyloidosis, shock lung, scleritis, sciatica, secondary adrenal insufficiency, silicone associated connective tissue disease, Sneddon-Wilkinson dermatosis, spondylitis ankylosans, Stevens-Johnson syndrome (SIS), systemic inflammatory response syndrome, temporal arteritis, toxoplasmic retinitis, toxic epidermal necrolysis, transverse myelitis, TRAPS (tumor necrosis factor receptor associated periodic syndrome), type 1 allergic reaction, type II diabetes, urticaria, usual interstitial pneumonia (VIP), vasculitis, vernal conjunctivitis, viral retinitis, Vogt-Koyanagi-Harada syndrome (VKH syndrome), wet macular degeneration, wound healing, yersinia or salmonella associated arthropathy.

Various receptor for advanced glycation endproducts (RAGE) diseases may be treated with the pharmaceutical compositions, viral particles, of the present disclosure. As a non-limiting example, the disease may be Amyotrophic Lateral Sclerosis, Brachial Plexus Injury, Brain Injury, including traumatic brain injury, Cerebral Palsy, Friedrich's Ataxia, Guillain Barre, Leukodystrophies, Multiple Sclerosis, Post Polio, Spina Bifida, Spinal Cord Injury, Spinal Muscle Atrophy, Spinal Tumors, Stroke, Transverse Myelitis, dementia, senile dementia, mild cognitive impairment, Alzheimer-related dementia, Huntington's chorea, tardive dyskinesia, hyperkinesias, manias, Morbus Parkinson, steel-Richard syndrome, Down's syndrome, myasthenia gravis, nerve trauma, vascular amyloidosis, cerebral hemorrhage I with amyloidosis, brain inflammation, Friedrich's ataxia, acute confusion disorder, amyotrophic lateral sclerosis, glaucoma, Alzheimer's disease, diabetic nephropathy, sepsis, rheumatoid arthritis and related inflammatory diseases.

Various neurite degenerative diseases may be treated with the pharmaceutical compositions, viral particles, of the present disclosure. As a non-limiting example, the disease may be multiple sclerosis, Parkinson's disease, Alzheimer's disease, Tay-Sachs disease, Niemann-Pick disease, Gaucher's disease, Hurler's syndrome, Huntington's disease, amyotrophic lateral sclerosis, idiopathic inflammatory demyelinating diseases, vitamin B12 deficiency, central pontine myelinolysis, tabes dorsalis, transverse myelitis, Devic's disease, progressive multifocal leukoencephalopathy, optic neuritis, traumatic injury to the CNS, an ischemic cerebral stroke, glaucoma, diabetic retinopathy, age-dependent macular degeneration, and a leukodystrophy.

Various neurological diseases may be treated with the pharmaceutical compositions, viral particles, of the present disclosure. As a non-limiting example, the disease may be Amyotrophic Lateral Sclerosis, Brachial Plexus Injury, Brain Injury, including traumatic brain injury, Cerebral Palsy, Guillain Barre, Leukodystrophies, Multiple Sclerosis, Post Polio, Spina Bifida, Spinal Cord Injury, Spinal Muscle Atrophy, Spinal Tumors, Stroke, Transverse Myelitis; dementia, senile dementia, mild cognitive impairment, Alzheimer-related dementia, Huntington's chorea, tardive dyskinesia, hyperkinesias, manias, Morbus Parkinson, steel-Richard syndrome, Down's syndrome, myasthenia gravis, nerve trauma, vascular amyloidosis, cerebral hemorrhage I with amyloidosis, brain inflammation, acute confusion disorder, amyotrophic lateral sclerosis, glaucoma and Alzheimer's disease.

Various cancers may be treated with pharmaceutical compositions, viral particles, of the present disclosure. As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus.

Types of carcinomas which may be treated with the viral particles of the present disclosure include, but are not limited to, papillomalcarcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma.

Types of sarcomas which may be treated with the viral particles of the present disclosure include, but are not limited to, soft tissue sarcoma such as alveolar soft part sarcoma, angiosarcoma, dermatofibrosarcoma., desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

As a non-limiting example, the cancer which may be treated may be Acute granulocytic leukemia, Acute lymphocytic leukemia, Acute myelogenous leukemia, Adenocarcinoma, Adenosarcoma, Adrenal cancer, Adrenocortical carcinoma, Anal cancer, Anaplastic astrocytoma, Angiosarcoma, Appendix cancer, Astrocytoma, Basal cell carcinoma, B-Cell lymphoma), Bile duct cancer, Bladder cancer, Bone cancer, Bowel cancer, Brain cancer, Brain stein glioma, Brain tumor, Breast cancer, Carcinoid tumors, Cervical cancer, Cholangiocarcinoma, Chondrosarcoma, Chronic lymphocytic leukemia, Chronic myelogenous leukemia, Colon cancer, Colorectal cancer, Craniopharyngioma, Cutaneous lymphoma, Cutaneous melanoma, Diffuse astrocytoma, Ductal carcinoma in situ, Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumors, General, Germ cell tumor, Glioblastoma multiforme, Glioma, Hairy cell leukemia, Head and neck cancer, Hemangioendothelioma, Hodgkin lymphoma, Hodgkin's disease, Hodgkin's lymphoma, Hypopharyngeal cancer, Infiltrating ductal carcinoma, Infiltrating lobular carcinoma, Inflammatory breast cancer, Intestinal Cancer, Intrahepatic bile duct cancer, Invasive/infiltrating breast cancer, Islet cell cancer, Jaw cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Nasal cavity cancer, Nasopharyngeal cancer, Neck cancer, Neuroblastoma, Neuroendocrine tumors, Non-Hodgkin lymphoma, Non-Hodgkin's lymphoma, Non-small cell lung cancer, Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Paget's disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumor, Pineoblastoma, Pituitary gland cancer, Primary central nervous system lymphoma, Prostate cancer, Rectal cancer, Renal cell cancer, Renal pelvis cancer, Rhabdomyosarcoma, Salivary gland cancer, Sarcoma, Sarcoma, bone, Sarcoma, soft tissue, Sarcoma, uterine, Sinus cancer, Skin cancer, Small cell lung cancer, Small intestine cancer, Soft tissue sarcoma, Spinal cancer, Spinal column cancer, Spinal cord cancer, Spinal tumor, Squamous cell carcinoma, Stomach cancer, Synovial sarcoma, T-cell lymphoma), Testicular cancer, Throat cancer, Thymoma/thymic carcinoma, Thyroid cancer, Tongue cancer, Tonsil cancer, Transitional cell cancer, Transitional cell cancer, Transitional cell cancer, Triple-negative breast cancer, Tubal cancer, Tubular carcinoma, Ureteral cancer, Ureteral cancer, Urethral cancer, Uterine adenocarcinoma, Uterine cancer, Uterine sarcoma, Vaginal cancer, and Vulvar cancer,

### Diagnostic Applications

The viral particles of the present disclosure may be used for diagnostic purposes or as diagnostic tools for any of the aforementioned diseases or disorders. As a non-limiting example, the viral particles of the present disclosure or the antibodies encoded within the viral genome therein may be used as a biomarker for disease diagnosis. As a second non-limiting example, the viral particles of the present disclosure or the antibodies encoded within the viral genome therein may be used for diagnostic imaging purposes, e.g., MRI, PET, CT or ultrasound.

### Preventative Applications

The viral particles of the present disclosure or the antibodies encoded by the viral genome therein may be used to prevent disease or stabilize the progression of disease. In some embodiments, the viral particles of the present disclosure are used to as a prophylactic to prevent a disease or disorder in the future. In some embodiments, the viral particles of the present disclosure are used to halt further progression of a disease or disorder. As a non-limiting example, the viral particles of the disclosure may be used in a manner similar to that of a vaccine.

### Research Applications

The viral particles of the present disclosure or the antibodies encoded by the viral genome therein may also be used as research tools. The viral particles of the disclosure may be used as in any research experiment, e.g., in vivo or in vitro experiments. In a non-limiting example, the viral particles of the disclosure may be used in cultured cells. The cultured cells may be derived from any origin known to one with skill in the art, and may be as non-limiting examples, derived from a stable cell line, an animal model or a human patient or control subject. In a non-limiting example, the viral particles of the disclosure may be used in in vivo experiments in animal models (i.e., mouse, rat, rabbit, dog, cat, non-human primate, guinea pig, ferret, c-elegans, drosophila, zebrafish, or any other animal used for research purposes, known in the art). In another non-limiting example, the viral particles of the disclosure may be used in human research experiments or human clinical trials.

### Combination Application

The viral particles of the disclosure may be used as a combination therapy with any other therapeutic molecule known in the art. The therapeutic molecule may be approved by the US Food and Drug Administration or may be in clinical trial or at the preclinical research stage. The therapeutic molecule may utilize any therapeutic modality known in the art, with non-limiting examples including gene silencing or interference (i.e., miRNA, siRNA, RNAi, shRNA), gene editing (i.e., TALEN, CRISPR/Cas9 systems, zinc finger nucleases), and gene, protein or enzyme replacement.

### Therapeutic Applications: Non-Infectious Disease

The present disclosure additionally provides a method for treating non-infectious diseases and/or disorders in a mammalian subject, including a human subject, comprising administering to the subject any of the viral particles or pharmaceutical compositions of the disclosure. In some embodiments, non-infectious diseases and/or disorders treated according to the methods described herein include, but are not limited to, Parkinson's Disease (PD), Dementia with Lewy Bodies (DLB), Multiple System Atrophy (MSA), Decreased muscle mass, Spinal muscular atrophy (SMA) Alzheimer's disease (AD), Amyotrophic lateral sclerosis (ALS), Huntington's Disease (HD), Multiple sclerosis (MS), Stroke, Migraine, Pain, Neuropathies, Psychiatric disorders including schizophrenia, bipolar disorder, and autism, Cancer, ocular diseases, systemic diseases of the blood, heart and bone, Immune system and Autoimmune diseases and Inflammatory diseases.

In some embodiments, methods of treating non-infectious diseases and/or disorders in a subject in need thereof may comprise the steps of (1) deriving, generating and/or selecting an antibody, antibody-based composition or fragment thereof that targets the antigen of interest; (2) producing a viral particle with a viral genome that includes a payload region encoding the selected antibody of (1); and (3) administering the viral particle (or pharmaceutical composition thereof) to the subject.

The present disclosure provides a method for administering to a subject in need thereof, including a human subject, a therapeutically effective amount of the viral particles of the disclosure to slow, stop or reverse disease progression. As a non-limiting example, disease progression may be measured by tests or diagnostic tool(s) known to those skilled in the art. As another non-limiting example, disease progression may be measured by change in the pathological features of the brain, CSF or other tissues of the subject.

### Parkinson's Disease

Parkinson's Disease (PD) is a progressive disorder of the nervous system affecting especially the substantia nigra of the brain. PD develops are a result of the loss of dopamine producing brain cells. Typical early symptoms of PD include shaking or trembling of a limb, e.g. hands, arms, legs, feet and face. Additional characteristic symptoms are stiffness of the limbs and torso, slow movement or an inability to move, impaired balance and coordination, cognitional changes, and psychiatric conditions e.g. depression and visual hallucinations. PD has both familial and idiopathic forms and it is suggestion to be involved with genetic and environmental causes. PD affects more than 4 million people worldwide. In the US, approximately 60, 000 cases are identified annually. Generally, PD begins at the age of 50 or older. An early-onset form of the condition begins at age younger than 50, and juvenile-onset PD begins before age of 20.

Death of dopamine producing brain cells related to PD has been associated with aggregation, deposition and dysfunction of alpha-synuclein protein (see, e.g. Marques and Outeiro, 2012, Cell Death Dis. 3:e350, Jenner, 1989, J Neural Neurosurg Psychiatry. Special Supplement, 22-28, and references therein). Studies have suggested that alpha-synuclein has a role in presynaptic signaling, membrane trafficking and regulation of dopamine release and transport. Alpha-synuclein aggregates, e.g. in forms of oligomers, have been suggested to be species responsible for neuronal dysfunction and death. Mutations of the alpha-synuclein gene (SNCA) have been identified in the familial forms of PD, but also environmental factors, e.g. neurotoxin affect alpha-synuclein aggregation. Other suggested causes of brain cell death in PD are dysfunction of proteasomal and lysosomal systems, reduced mitochondrial activity.

PD is related to other diseases related to alpha-synuclein aggregation, referred to as “synucleinopathies.” Such diseases include, but are not limited to, Parkinson's Disease Dementia (PDD), multiple system atrophy (MSA), dementia with Lewy bodies, juvenile-onset generalized neuroaxonal dystrophy (Hallervorden-Spatz disease), pure autonomic failure (PAF), neurodegeneration with brain iron accumulation type-4 (NBIA-1) and combined Alzheimer's and Parkinson's disease.

As of today, no cure or prevention therapy for PD has been identified. A variety of drug therapies available provide relief to the symptoms. Non-limiting examples of symptomatic medical treatments include carbidopa and levodopa combination reducing stiffness and slow movement, and anticholinergics to reduce trembling and stiffness. Other optional therapies include e.g. deep brain stimulation and surgery. There remains a need for therapy affecting the underlying pathophysiology. For example, antibodies targeting alpha-synuclein protein, or other proteins relevant for brain cell death in PD, may be used to prevent and/or treat PD.

In some embodiments, methods of the present disclosure may be used to treat subjects suffering from PD and other synucleinopathies. In some cases, methods of the present disclosure may be used to treat subjects suspected of developing PD and other synucleinopathies.

AAV Particles and methods of using the viral particles described in the present disclosure may be used to prevent, manage and/or treat PD. As a non-limiting example, the viral particles of the present disclosure comprise a nucleic acid sequence encoding at least one of the sequences described in Table 3.

Dementia with Lewy Bodies

Dementia with Lewy Bodies (DLB), also known as diffuse Lewy body disease, is a form of progressive dementia, characterized by cognitive decline, fluctuating alertness and attention, visual hallucinations and parkinsonian motor symptoms. DLB may be inherited by an autosomal dominant pattern. DLB affects more than 1 million individuals in the US. The condition typically shows symptoms at the age of 50 or older.

DLB is caused by the abnormal build-up of Lewy bodies, aggregates of the alpha-synuclein protein, in the cytoplasm of neurons in the brain areas controlling memory and motor control. The pathophysiology of these aggregates is very similar to aggregates observed in Parkinson's disease and DLB also has similarities to Alzheimer's disease. Inherited DLB has been associated with gene mutations in SNCA and SNOB genes, producing synuclein proteins.

As of today, there is no cure or prevention therapy for DLB. A variety of drug therapies available are aimed at managing the cognitive, psychiatric and motor control symptoms of the condition. Non-limiting examples of symptomatic medical treatments include e.g. acetylcholinesterase inhibitors to reduce cognitive symptoms, and levodopa to reduce stiffness and loss of movement. There remains a need for therapy affecting the underlying pathophysiology. Antibodies targeting alpha-synuclein protein may be used to prevent and/or treat DLB.

In some embodiments, methods of the present disclosure may be used to treat subjects suffering from DLB. In some cases, methods of the present disclosure may be used to treat subjects suspected of developing DLB.

AAV Particles and methods of using the viral particles described in the present disclosure may be used to prevent, manage and/or treat DLB. As a non-limiting example, the viral particles of the present disclosure comprise a nucleic acid sequence encoding at least one of the sequences described in Table 3 (SEQ ID NO: 2948-17938).

### Multiple System Atrophy

Multiple system atrophy (MSA), also known as Shy-Drager Syndrome, is a progressive neurodegenerative disorder. The characteristic symptoms are associated with failure of autonomic nervous system causing dizziness, fainting, bladder control problems, and problems regulating heart rate, blood pressure and breathing, accompanied by motor control symptoms similar to Parkinson's disease, e.g. tremor, rigidity and loss of muscle coordination. The symptoms are a reflection of the loss of nerve cells in certain areas of the brain and spinal cord. The disease typically develops around ages of 50 or 60 years. MSA affects approximately 50,000 individuals in the US.

MSA belongs to the synucleinopathies and is characterized by the appearance of glial cytoplasmic inclusions (GCIs) in oligodendrocytes, which are the myelin producing support cells of the central nervous system (see, e.g. Bleasel et al. 2014, Acta Neuropathologica Communications, 2014, 2:15, and references therein). GCIs comprise insoluble proteinaceous filaments composed of the alpha-synuclein protein. Also, tau proteins have been identified in GCIs. The pathophysiology of the CGIs is not yet fully understood but alpha-synuclein and tau proteins are suggested to have a role in the development and progression of SMA.

As of today, there is no cure or prevention therapy for MSA. A variety of drug therapies available are aimed at managing the symptoms. Non-limiting examples of symptomatic medical treatments include those used for Parkinson's disease to relief symptoms related motor movement, increased salt intake and steroid hormones for increasing blood pressure. There remains a need for therapy affecting the underlying pathophysiology. Antibodies targeting tau and alpha-synuclein proteins may be used to prevent and/or treat MSA.

In some embodiments, methods of the present disclosure may be used to treat subjects suffering from MSA. In some cases, methods of the present disclosure may be used to treat subjects suspected of developing MSA.

AAV Particles and methods of using the viral particles described in the present disclosure may be used to prevent, manage and/or treat MSA. As a non-limiting example, the viral particles of the present disclosure comprise a nucleic acid sequence encoding at least one of the sequences described in Table 3 or Table 6.

### Decreased Muscle Mass, Muscle Strength and Muscle Function

A number of diseases, disorders and condition are associated with muscle weakness, which refers to reduced muscle mass, muscle strength and muscle function. For example, such disorders include myopathies, which are neuromuscular disorders characterized by muscle weakness due to dysfunction of muscle fiber. Myopathies include, but are not limited to, congenital myopathies, muscular dystrophies, mitochondrial myopathies, glycogen storage diseases of muscle, myoglobinurias, dermatomyositis, myositis ossificans, familial periodic paralysis, polymyositis, inclusion body myositis, and related myopathies, neuromyotonia, stiff-man syndrome, common muscle cramps and stiffness, and tetany. Muscle weakness may also be caused by ageing, diabetes, obesity, chronic pain, peripheral vascular disease, chronic lung diseases, heart diseases, cancers, anemia, arthritis, chronic renal failure and renal diseases, chronic obstructive pulmonary disease, multiple sclerosis (MS), stroke, muscular dystrophy, motor neuron neuropathy, amyotrophic lateral sclerosis (ALS), Parkinson's disease, osteoporosis, osteoarthritis, fatty acid liver disease, liver cirrhosis, Addison's disease, Cushing's syndrome, acute respiratory distress syndrome, steroid induced muscle wasting, myositis, scoliosis, or infections e.g influenza, Epstein-Barr virus infection, HIV/AIDS, Lyme disease, and hepatitis C infection, Muscle weakness may occur after surgery, burn trauma, medical treatment, or trauma. through an injury. Severity of muscle weakness varies. In many cases the condition reduces the quality of life significantly or may be even life-threatening.

A regulator protein associated with muscles is myostatin (MSTN), also known as growth and differentiation factor 8 (GDF-8). Myostatin is a protein encoded by the MSTN gene, released in the myocytes. Myostatin and myostatin receptors (e.g. ACVR2A and ACVR2B), have a role in suppressing the growth and development of muscle tissue in the body.

Treatment of muscle weakness depends on the underlying disease or condition, and may include e.g. drug therapy, good nutrition, physiotherapy, mechanical support for weakened muscles and/or surgery. However, efficient therapy to treat a combination of loss of muscle mass, muscle strength and muscle function are needed. Antibodies targeting myostatin may be used in the treatment and prophylaxis of diseases associated with such conditions. For example, bimagrumab (developed by Novartis) is a monoclonal antibody targeting ACVR2B myostatin receptor and used for therapy of musculoskeletal diseases and domagrozumab (developed by Pfizer) is an antibody targeting myostatin, and used for therapy of muscle degeneration and muscle weakness.

In some embodiments, methods of the present disclosure may be used to treat subjects suffering from loss of muscle mass, muscle strength and muscle function. In some cases, methods of the present disclosure may be used to treat subjects suspected of developing such conditions.

AAV Particles and methods of using the viral particles described in the present disclosure may be used to prevent, manage and/or treat MSA. As a non-limiting example, the viral particles of the present disclosure comprise a nucleic acid sequence encoding at least one of the sequences described in Table 6.

### Spinal Muscular Atrophy

Spinal muscular atrophy (SMA) is a hereditary disease-causing weakness and wasting of the voluntary muscles in the arms and legs of infants and children. SMA is associated with abnormalities in the protein production of the survival motor neuron gene 1 (SMN1). Lack of the protein affects degeneration and death of lower motor neurons. Typical symptoms include floppy limbs and trunk, feeble movement of the arms and legs, difficulties in swallowing and eating, and impaired breathing. SMA is the most common genetic disorder leading to death of children under 2 years of age. SMA affects one in 6,000 to 10,000 people.

As of today, there is no cure for SMA. Therapies available are aimed at management of the symptoms and prevention of additional complications. Such therapies are associated e.g. with cardiology, movement management, respiratory care and mental health. There remains a need for therapy affecting the underlying pathophysiology of SMA.

In some embodiments, methods of the present disclosure may be used to treat subjects suffering from SMA. In some cases, methods of the present disclosure may be used to treat subjects suspected of developing SMA.

AAV Particles and methods of using the viral particles described in the present disclosure may be used to prevent, manage and/or treat SMA. As a non-limiting example, the viral particles of the present disclosure comprise a nucleic acid sequence encoding at least one of the sequences described in Table 6.

### Alzheimer's Disease

Alzheimer's Disease (AD) is a debilitating neurodegenerative disease and the most common form of dementia affecting the memory, thinking and behavior. Typical early symptom is difficulty of remembering newly learned information. As the disease advances, symptoms include disorientation, changes in sleep, changes in mood and behavior, confusion, unfound suspicions and eventually difficulty to speak, swallow and walk. AD currently afflicts more than 35 million people worldwide, with that number expected to double in coming decades.

As of today, no cure or prevention therapy for AD has been identified. Drug therapy to treat memory loss, behavioral changes and sleep changes, and to slow down the progression of AD are available. However, these symptomatic treatments do not address the underlying pathophysiology.

The AD brain is characterized by dual aggregates, the extracellular β-amyloid plaques and the intracellular neurofibrillary tangles (NTT) of misfolded, hyperphosphorylated microtubule associated, tau proteins. The P-amyloid plaques may lead to pathological cascades that are associated with a number of proteins, such as, but not limited to, APP (amyloid beta (A4) precursor protein), A beta (amyloid beta), BALE (Beta-secretases), and APOE (apolipoprotein E). Historically, it has been thought that amyloid pathology precedes the appearance of NFT, and therefore, that tau pathology in the form of aggregates is symbolic of impending cell death (Selkoe, D. J., 2001, Physiological Reviews, 81(2):741-66). However, clinical trials addressing amyloid pathology have largely failed thus far and advances in the field suggest that targeting tau aggregates may be advantageous and lead to improved cognitive ability.

In some embodiments, methods of the present disclosure may be used to treat subjects suffering from AD. In some cases, methods of the present disclosure may be used to treat subjects suspected of developing AD.

AAV Particles and methods of using the viral particles described in the present disclosure may be used to prevent, manage and/or treat AD. As a non-limiting example, the viral particles of the present disclosure comprise a nucleic acid sequence encoding at least one of the sequences described in Table 4.

### Huntington's Disease

Huntington's disease (FID) is a rare, inherited disorder causing degeneration of neurons in the motor control region of the brain, as well as other areas. Typical symptoms of the disease include uncontrolled movements (chorea), abnormal postures, impaired coordination, slurred speech and difficulty of feeding and swallowing accompanied by changes in behavior, judgment and cognition. HD is caused by mutations in the gene associated with the huntingtin (HTT) protein. The mutation causes the (CAG) blocks of DNA. to repeat abnormally many times. HD affects approximately 30, 000 individuals in the US.

HD is characterized by mutations of the huntingtin (HTT) protein with abnormal expansions of polyglutamine tracts, e.g. expansion of the length of glutamine residues encoded by CAG repeats. The expansion threshold for occurrence of the disease is considered to be approximately 35-40 residues. HD is also associated with beta sheet rich aggregates in striatal neurons formed by N-terminal region of HIT. The expansions and aggregates lead to gradual loss of neurons as HD progresses. Additionally, the cell death in HD is associated with death receptor 6 (DR6) which is known to induce apoptosis.

As of today, there is no therapy to cure, or prevent the progression of the disease. Drug therapies available are aimed at management of the symptoms. For example, FDA has approved tetrabenezine to be prescribed for prevention of chorea. Additionally, e.g. antipsychotic drugs may help to control delusions, hallucinations and violent outbursts. There remains a need for therapy affecting the underlying pathophysiology, such as antibody therapies targeting the HTT protein, DR6 protein, and/or other HD associated proteins.

In some embodiments, methods of the present disclosure may be used to treat subjects suffering from HD. In some cases, methods of the present disclosure may be used to treat subjects suspected of developing HD.

AAV Particles and methods of using the viral particles described in the present disclosure may be used to prevent, manage and/or treat HD. As a non-limiting example, the viral particles of the present disclosure comprise a nucleic acid sequence encoding at least one of the sequences described in Table 5.

### Multiple Sclerosis

Multiple sclerosis is a disease of the central nervous system (CNS). The typical early symptoms occurring between the ages of 20 to 40 include blurring vision, red-green color distortion, partial blindness, extreme muscle weakness, feeling of numbness or prickling, difficulties with coordination and balance. In severe cases MS may lead to a partial or complete paralysis. MS is believed to be an autoimmune disease as the communication between the brain and other parts of the body being disrupted as the immune system causes an inflammation within the central nervous system. MS is caused by both genetic and environmental factors, e.g. viral infections. MS is the most common neurological condition of young adults globally, affecting more than 2.3 million individuals.

At present time, the pathophysiology of MS is not fully understood. The disease is associated with a complex combination related to formation of lesions in the central nervous system, inflammation and demyelination (destruction of the protective myelin surrounding the nerve fibers) in white matter and cortex, and axon destruction (see, e.g. Longbrake et at, 2013, Curr Neurol Neurosci Rep., 13(11), and references therein). A number of myelin inhibitory proteins have been characterized in association with MS, including, but not limited to, NogoA ((Neurite outgrowth inhibitor A), Nogo receptor-1 (NgR1), myelin associated glycoprotein (MAG), oligodendrocyte glycoprotein (OM-gp), LINGO-1 (Leucine rich repeat and immunoglobin-like domain-containing protein 1), and MAI (myelin associated inhibitor). MS is also affiliated with many immune response related proteins. Non-limiting examples of such proteins include e.g. B-cell and T-cell associated proteins, such as, but not limited to, leukocyte surface antigen CD52, alpha chain of the IL-2 receptor CD25, B-cell surface molecule CD20, T helper cell CD4, and/or cytokine IL-12/23. Alpha 4-integrin, has been associated with inflammation of CNS, as it has a role in leukocyte adhesion and migration to the inflamed CNS. Additionally, MS patients have been characterized with elevated tumor necrosis factor (TNF) level s.

As of today, there is no prevention therapy or cure for MS. Patients in need of medical therapy may be treated with e.g. synthetic form of myelin basic protein, (Copaxone, copolymer I), antiviral proteins known as interferons, or immunosuppressant drugs e.g. mitoxantore. Some drugs are aimed at treating a symptom of MS, such as dalapridine, which is aimed at improving walking of individuals with MS. Antibodies for MS have been developed. For example, natalizumab is a monoclonal antibody targeting alpha 4 integrin, (developed by Elan Pharmaceuticals and Biogen) approved by the FDA for treatment of relapsing MS under treatment guidelines to monitor patients by physicians. Other non-limiting examples for MS antibody drugs include alemtuzumab (CD52), daclizumab (CD25), rituximab (CD20), ocrelizumab (CD20), ofatumumab (CD20), (see, e.g. Longbrake et al., 2013, Curr Neural Neurosci Rep., 13(11), and references therein). However, many current medications have serious side effects, and there remains a need for therapy affecting the underlying pathophysiology, such as improved antibody therapies.

In some embodiments, methods of the present disclosure may be used to treat subjects suffering from MS. In some cases, methods of the present disclosure may be used to treat subjects suspected of developing MS.

AAV Particles and methods of using the viral particles described in the present disclosure may be used to prevent, manage and/or treat MS. As a non-limiting example, the viral particles of the present disclosure comprise a nucleic acid sequence encoding at least one of the sequences described in Table 6,

### Amyotrophic Lateral Sclerosis

Amyotrophic Lateral Sclerosis (ALS), also known as Lou Gehrig's disease or classical motor neuron disease, is a rapidly progressive and fatal neurological disease. ALS is associated with cell degeneration and death of the upper and lower motor neurons, leading to enablement of muscle movement, weakening, wasting and loss of control over voluntary muscle movement. Early symptoms include muscle weakness of hands, legs and swallowing muscles, eventually progressing to inability to breathe due to diaphragm failure. According to Centers for Disease Control and Prevention (CDC), ALS affects an estimated 12,000-15,000 individuals in the US. About 5-10% of cases are familial.

ALS, as other non-infectious neurodegenerative diseases, has been characterized by presence of misfolded proteins, including, but not limited to, tau, amyloid-beta (A beta), alpha-synuclein, HTT (huntingtin) or SOD1 (superoxide dismutase 1 protein), and myelin associated inhibitors and their receptors, (see, e.g., Krishnamurthy and Sigurdsson, 2011, N Biotechnol. 28(5):511-7, and Musaro, 2013, HMS J; 280(17):4315-22, and references therein). Familial ALS has been associated with mutations of TAR DNA-binding protein 43 (TDP-43) and RNA-binding protein FUS/ILS. Some proteins have been identified to slow down progression of ALS, such as, but not limited, to growth factors, e.g. insulin-like growth factor 1 (IGF-1), glial cell line-derived growth factor, brain-derived growth factor, vascular endothelial growth factor and ciliary neurotrophic factor, or growth factors promoting muscle growth, e.g. myostatin.

As of today, there is no prevention or cure for ALS. FDA approved drug niluzole has been approved to prolong the life, but does not have an effect on symptoms. Additionally, drugs and medical devices are available to tolerate pain and attacks associated with ALS. There remains a need for therapy affecting the underlying pathophysiology.

In some embodiments, methods of the present disclosure may be used to treat subjects suffering from ALS. In some cases, methods of the present disclosure may be used to treat subjects suspected of developing AT S.

AAV Particles and methods of using the viral particles described in the present disclosure may be used to prevent, manage and/or treat MS. As a non--limiting example, the viral particles of the present disclosure comprise a nucleic acid sequence encoding at least one of the sequences described in Table 6.

### Stroke

Stroke is a medical emergency characterized by a burst of a blood vessel in the brain, referred to as hemorrhagic stroke, or an interruption of blood supply in the brain, referred to as ischemic stroke. Stroke triggers an inflammation, and causes brain cell death, as the oxygen and nutrient supply is impaired suddenly. Typical symptoms include numbness or weakness, especially on one side of the body, confusion, trouble speaking and understanding speech, vision problems, dizziness and loss of balance. Typically, patients recovering from stroke have permanent disabilities, e.g. affecting movement, speech, coordination, vision and balance. Medical conditions, e.g. diabetes, high blood pressure, high cholesterol, and obesity, as well as, cigarette smoking and poor nutrition, increase susceptibility to a stroke. According to CDC, stroke affects about 800,000 people in the US annually and is the fifth most common cause of death.

Typical recovery from a stroke is slow or impartial. The inability of the central nervous system to repair after injury has been associated with inhibitory proteins associated with CNS. For example, myelin associated proteins, such as, but not limited to, myelin associated glycoprotein (MAG), myelin associated inhibitor (MAI), and their receptors, proteoglycans, versi can V2, oligodendrocyte myelin glycoprotein (Omgp), and neurite outgrowth inhibitor (logo) have been identified to inhibit neurite outgrowth (see, e.g. Yu et al., 2013, Transl Stroke Res, 4(5):477-83, and references therein). Cell death in ischemic stroke has been associated with excessive activation of glutamate receptors, involved with glutamate receptors such as, but not limited to, N-methyl-D-aspartic acid (NMDA) receptors and DL-a-amino-3-hydroxy-5 ethyl-4-i soxazole propionic acid (AMPA). Inflammatory signaling triggered after stroke has been associated with adhesion molecules of the endothelial cells, such as, but not limited to, selectin family, intercellular adhesion molecule-1 (ICAM-1, also known as CD54), and 132-integrins.

Therapies to prevent stroke are typically focused on treatment of underlying medical conditions. Acute treatment after stroke involves dissolving blood clot in the case of an ischemic stroke es. by antiplatelet agents, anticoagulants and thrombolytics, or quenching of bleeding in the case of a hemorrhagic stroke. As of today, there is no effective prevention therapy for a stroke. There remains a need for therapy affecting the underlying pathophysiology of a stroke. Antibodies targeting the stroke associated proteins have been developed. For example, Refanezumab is a monoclonal antibody targeting myelin-associated glycoprotein, MAG, for improvement and recovery of motor function after stroke.

In some embodiments, methods of the present disclosure may be used to prevent a stroke, or treat individuals recovering from a stroke.

AAV Particles and methods of using the viral particles described in the present disclosure may be used to prevent, manage and/or treat stroke. As a non-limiting example, the viral particles of the present disclosure comprise a nucleic acid sequence encoding at least one of the sequences described herein.

### Migraine

Migraine is a neurological condition characterized by reoccurring attacks of severe headache, accompanied by nausea, light visions, and sensitivity to light, sound and movement. Migraine attacks may last from hours to days. The cause of migraine is unknown, but it is associated with some underlying diseases, as well as environmental and genetic factors. Migraine affects about 12% of population in the US.

Present methods for management and treatment of migraine include medical therapies (e.g. analgesics, triptans, ergotamines), surgery, and neurostimulation. As of today, there is no therapy to prevent or cure migraine, and a need for medical therapy focusing on the pathophysiology of migraine remains. CGRP (calcitonin gene-related peptide) vasodilatation has been associated with migraine and photophobia, which is a typical symptom of a migraine attach. Antibodies targeting CGRP may be used for treatment and/or management of migraine, e.g. as described in U.S. Pat. Nos. 9,115,194, and 9,102,731, and US Patent application US20120294802, the contents of which are herein incorporated by reference in their entirety.

In some embodiments, methods of the present disclosure may be used to treat subjects suffering from migraine. In some cases, methods of the present disclosure may be used to treat subjects suspected of developing migraine.

AAV Particles and methods of using the viral particles described in the present disclosure may be used to prevent, manage and/or treat migraine. As a non-limiting example, the viral particles of the present disclosure comprise a nucleic acid sequence encoding at least one of the sequences described in Table 10.

### Pain

Pain is a complex symptom associated with a variety of diseases and disorders and may be acute or chronic. Pain is challenging to treat, and many anti-pain medications have side effects, and/or they can be addictive. There remains a need for pain medications affecting the underlying pathophysiology of a pain. Antibodies for treatment for pain are on the market. For example, fasinumab (developed by Regeneron Pharmaceuticals Inc.), Fulranumab (developed by Johnson & Johnson) and tanezumab (developed by Pfizer) are antibodies against NGF (nerve growth factor) for treatment of pain, such as, osteoarthritis knee pain, chronic low back pain, bone cancer pain and/or pain associated with interstitial cystitis.

In some embodiments, methods of the present disclosure may be used to treat subjects suffering from pain. In some cases, methods of the present disclosure may be used to treat subjects suspected of developing pain.

AAV Particles and methods of using the viral particles described in the present disclosure may be used to prevent, manage and/or treat pain. As a non-limiting example, the viral particles of the present disclosure comprise a nucleic acid sequence encoding at least one of the sequences described in Table 10.

### Neuropathies

Neuropathies are a group of diseases or conditions affecting the nerves. Typical symptoms of neuropathies include impaired movement and sensation, cramping, pain and abnormal organ functions. Neuropathies include e.g. diabetic neuropathy, cisplatin-induced neuropathy, mononeuropathy, pyridoxine-induced neuropathy, peripheral neuropathy, small fiber peripheral neuropathy, polyneuropathy and cisplatin/pyridoxine-induced neuropathy.

As of today, there is no prevention or treatment therapy specific for neuropathies on the market. Typical treatment involves with treatment of underlying diseases, e.g. diabetes, or management of the symptoms. Therefore, there remains a need for therapy affecting the underlying pathophysiology of neuropathi es, such as efficient antibody therapies. Tyrosine kinases, such as Trk receptors, have a role in regulation of the nervous system, neuronal survival and signal cascades. Antibodies targeting e.g. Trk C may be used for prevention, treatment and/or management of neuropathies, as described in U.S. Pat. No. 7,615,383, the contents of which are herein incorporated by reference in their entirety.

In some embodiments, methods of the present disclosure may be used to treat subjects suffering from neuropathies. In some cases, methods of the present disclosure may be used to treat subjects suspected of developing neuropathies.

AAV Particles and methods of using the viral particles described in the present disclosure may be used to prevent, manage and/or treat neuropathies. As a non-limiting example, the viral particles of the present disclosure comprise a nucleic acid sequence encoding at least one of the sequences described in Table 7.

### Psychiatric Disorders

Psychiatric disorders are characterized by behavioral or mental condition that affects individual's ordinary ability to function. Common psychiatric disorders include, but are not limited to, Tourette syndrome, bipolar disorder, schizophrenia, anxiety, depression, panic disorder, obsessive-compulsive disorder (0CD), eating disorders (e.g. anorexia, bulimia, orthorexia, obesity), substance abuse (e.g. alcohol or drug), addiction, psychosis, phobias, mood disorders, manic-depression disorder, insomnia and other sleep disorders. Psychiatric disorders may significantly affect individual's quality of life, and in severe cases lead to harmful behavior, such as suicidal or homicidal behavior. The diseases are typically managed and treated with psychotherapy, behavioral therapy, medical therapy (e.g. antipsychotic drugs), and/or other therapies. There remains a need for improved medical therapies affecting the underlying pathophysiology of psychiatric disorders, such as antibodies targeting proteins associated with such disorders.

For example, ghrelin hormone has been associated with eating disorders, including obesity and anorexia. Antibodies targeting ghrelin may be used for prevention, management and/or treatment of eating disorders, e.g. as described in US Patent application US20060233788, the contents of which are herein incorporated by reference in their entirety.

Depression has been associated with an inhibition of peripheral cytokine activity, especially INFa (tumor necrosis factor alpha). Antibodies targeting TNF alpha may be used for prevention, management and/or treatment of depression, e.g. as described in U.S. patent application US20140296493, the contents of which are herein incorporated by reference in their entirety.

OCD and OCD related diseases have been associated T-cell activation. Anx-A1 (annexin A1) is a protein promoting T-cell activation, and antibodies binding Annexin-1 may be used for prevention, management and/or treatment of OCD and related diseases, e.g. as described in US Patent application US20150004164, the contents of which are herein incorporated by reference in their entirety.

In some embodiments, methods of the present disclosure may be used to treat subjects suffering from a psychiatric disorder. In some cases, methods of the present disclosure may be used to treat subjects suspected of developing a psychiatric disorder.

AAV Particles and methods of using the viral particles described in the present disclosure may be used to prevent, manage and/or treat psychiatric disorder. As a non-limiting example, the viral particles of the present disclosure comprise a nucleic acid sequence encoding at least one of the sequences described in Table 8.

### Cancer

Cancer is a group of more than 100 diseases associated with abnormal division and cell growth with characteristic spreading in the body. Many cancers are in the form of tumors, e.g. breast cancer, lung cancer, colon cancer, ovarian cancer, renal cancer, prostate cancer, head and neck cancer, pancreas cancer, bone cancer, and thyroid cancer. Cancers associated with blood and lymphoid tissues may be referred to as liquid tumors, e.g. leukemia, lymphoma and myeloma. Cancer is caused by failure of tissue growth regulation. Genes associated with cancer include oncogenes, that promote cell growth and reproduction, and tumor suppressor genes, that inhibit cell division. Oncogenes include, but are not limited to, growth factors, receptor and cytoplasmic tyrosine kinases, transcription factors, serine/threonine kinases and regulatory GTPases. Tumor protein pS3 is the most common tumor suppressor protein found in more than half of cancer types. Susceptibility to cancer is involved with environmental factors, as well as genetic. Though progress with prevention, diagnosis and treatment of cancer has been tremendous, cancer still remains a severe and life-threatening disease. According to American Cancer Society, an estimated 1.6 cancers are diagnosed annually in the US, leading to more than a half a million deaths.

Therapies associated with cancer treatment include surgery, chemotherapy, radiation and antibody therapies. Antibodies for treatment and/or prevention of cancers have been on the market for nearly two decades, and are considered as one of the most important strategies for treatment of e.g. hematological malignancies and solid tumors. A number of cancer-associated antigens have been identified for treatment of cancers. Antibodies targeting such antigens may be used to diagnose, prevent and/or treat the associated cancers (see, e.g. Scott et al, 2012, Nature Reviews Cancer 12, 278-287, and references therein).

Some solid cancer tumors are associated with expressed glycoproteins antigens. Such antigens include, but are not limited to, EPCAM (Epithelial cell adhesion molecule), CEA (Carcinoembryonic antigen), gpA33 (Glycoprotein A33 (Transmembrane)), mucins, TAG-72 (Tumor-associated glycoprotein 72), CAIX (Carbonic anhydrase IX), PSMA (Prostate-specific membrane antigen), and FBP (Folate-binding protein). Antibodies targeting the expressed glycoproteins may be used to treat associated tumors. Such solid tumors include, but are not limited to, breast, colon cancer, lung, colorectal, ovarian, renal cell, and/or prostate tumors.

Some solid cancer tumors are associated with growth factor and differentiation signaling associated antigens. Such antigens include, but are not limited to, EGFR/ERBB 1/HER I (epidermal growth factor receptor 1), ERBB2 (epidermal growth factor receptor 2), ERBB3 (epidermal growth factor receptor 3), MET (Tyrosine-Protein Kinase Met), IGF1R (insulin-like growth factor 1 receptor), EPHA3 (EPH Receptor A3), TRAILR1, (Death receptor 4), and (Receptor activator of nuclear factor kappa-B ligand). Cancers that may be treated with antibodies targeting the growth factor and differentiation signaling include, but are not limited to, breast, colon, lung, ovarian, prostate, head and neck, pancreas, thyroid, kidney, and colon tumors, melanoma, glioma, bone metastases, and hematological malignancies.

Some cancer tumors are associated with antigens of stromal and extracellular matrix. Such antigens include, but are not limited to, tenascin and FAP (Fibroblast Activation Protein, Alpha). Cancers that may be treated with antibodies targeting the stromal and extracellular matrix antibodies include, but are not limited to, breast, prostate, colon, lung, pancreas and head and neck tumors and glioma.

Some cancer tumors are associated with such as Lewis-Y Le(y) antigen. Le(y) antigen has been found expressed on a number of cancers, such as, but not limited to, ovarian, breast, colon, lung and prostate cancer. Antibodies targeting Le(y) antigen may be used to treat the associated cancers.

Some cancer tumors are associated with glycolipid antigens. Such antigens include, but are not limited to, gangliosides, such as GD2, GD3, and GM2 (monosialotetrahexosylganglioside 2). Cancers that may be treated with antibodies targeting the glycolipid antigens include, but are not limited to, epithelial tumors (e.g. breast, colon and lung tumors) and neuroectodermal tumors (tumors of the central and peripheral nervous system).

The vasculature of solid tumors is abnormal, compared to normal vasculature. Antigens supporting the formation of abnormal microvasculature and progress of cancer include, but are not limited to, VEGF (Vascular endothelial growth factor), VEGFR (vascular endothelial growth factor receptor), integrin αVβ3 and integrin α5β1. Antibodies targeting such antigens may be used to treat a number of solid tumors such as, but not limited to, lung, breast, renal, brain, eye, colorectal, melanoma, ovarian, and/or other tumors, by preventing the formation of abnormal vasculature.

Hematopoietic and lymphoid malignancies are cancers affecting the blood, bone marrow, lymphs and lymphatic system. Such cancers include e.g. leukemias (acute and chronic lymphoblastic leukemia, acute and chronic myelogenous leukemia), lymphomas (Hodgkin's lymphoma, Non-Hodgkin's lymphoma) and myelomas. Tumors of the hematopoietic and lymphoid tissues are closely related to immune systems. Hematological tumors may be caused by chromosomal abnormalities derived from the myeloid and lymphoid cell lines. The lymphoid cell line produces T and B cells, whereas myeloid cell line produces granulocytes, erythrocytes, thrombocytes, macrophages and mast cells. T and B cell associated hematopoietic differentiation antigens are glycoproteins that are usually from cluster of differentiation (CD) group, such as, but not limited to, CD20, CD30, CD33 and CD52. Antibodies targeting such antigens may be used for prevention and/or treatment of hematopoietic and lymphoid cancers.

In some embodiments, methods of the present disclosure may be used to treat subjects suffering from a cancer. In some cases, methods of the present disclosure may be used to treat subjects suspected of developing a cancer.

In some embodiments, methods of the present disclosure may be used for immuno-oncology (I-O) applications. viral particles or pharmaceutical compositions of the present disclosure may be used to develop an immunotherapy or as an immunotherapy in an I-O treatment of a subject suffering from cancer. Non-limiting examples of I-O applications include active, passive or hybrid immunotherapies, checkpoint blockade, adoptive cell transfer (ACT), cancer vaccines, CAR or CAR-T therapies, dendritic cell therapy, stem cell therapies, natural killer (NK) cell-based therapies, and interferon or interleukin based methods.

AAV Particles and methods of using the viral particles described in the present disclosure may be used to prevent, manage and/or treat cancer. As a non-limiting example, the viral particles of the present disclosure comprise a nucleic acid sequence encoding at least one of the sequences described in Table 9.

### Ocular Diseases

Eye is an organ comprising a number of components, including the cornea, aqueous humor, lens, vitreous humor, retina, the retinal pigment epithelium, and choroid. Ocular diseases are conditions affecting the different tissues of the eye. A number of diseases and disorders affect the different components of the eye, and may cause impaired vision, full or partial blindness, irritation, dryness, sensitivity, photophobia, and/or light aversion.

Complement in the eye has an important role in protecting the eye from infections and in modulation of the immune and inflammatory responses. In normal eye, the complement activity is at low level and is regulated by membrane bound and soluble intraocular complement regulatory proteins. Disturbance of the balance between complement activation and complement inhibition may lead to damage to self-tissue (see, e.g, ha et al., 2007, Mol Immunol.; 44(16): 3901-3908, and references therein). The complement system may be activated in three pathways. The classical pathway is activated by immune complexes or substances and involves e.g. complement components C1, C2, C3, C4, C3a, C5, C5a, C5b, CO, C7, C8, C9 and C5b-9. The alternative pathway activates complement component C3 when in interaction with e.g. zymosan, or lipopolysaccharide surfaces, additionally involving, e.g. Factor B, Factor Ba, Factor Bb, Factor D, and Factor P. The third activation pathway is the lectin pathway, and is related to interaction of certain serum lectins, e.g. mannose binding lectin (MBL), mannose and N-acetyl glucosamine residues present in bacterial cell walls. Complement activation is associated with a number of ocular diseases, such as, but not related, age-related macular degeneration (AMD); diabetic retinopathy, choroidal neovascularization (CNV), uveitis, diabetic macular edema, pathological myopia, von Hippel-Lindau disease, histoplasmosis of the eye, Central Retinal Vein Occlusion (C QVC)), corneal neovascularization, and retinal neovascularization, choroidal neovascularization, and other ocular conditions involving complement activation. Antibodies targeting the associated complement components may be used to diagnose, manage and/or treat such ocular diseases.

Age-related macular degeneration (AMD) is a major cause of irreversible loss of central vision in the elderly worldwide. AMD leads to gradually worsening vision. AMI) does not result in blindness, but may affect daily life. Wet AMD is caused by abnormal blood vessels behind the retina grow under the macula and leak blood and fluid that damage the macula. Wet AMD may be treated with laser coagulation and medication to reverse or stop the growth of blood vessels. Dry AMD is caused by break down of the light sensitive cells in the macula. As of today, there is no treatment for dry AMD.

There remains a need for prevention, management and treatment therapies for wet and dry AMD. AMD is associated with complement components, as described above. In addition, AMD is associated with proteins such as, but not limited to, VEGF (Vascular endothelial growth factor), EPO (Erythropoietin), EPOR (EPC) receptor), Interleukins IL-1p, IL-17A, Il-10, TNFo. (tumor necrosis factor alpha), or FGFR2 (Fibroblast Growth Factor Receptor). Antibodies targeting the AMD associated complement and growth proteins may be used to treat AN/ID. For example, bevacizumab and ranibizumab (developed by Genentech Inc.) are antibodies targeting VEGF-A to slow down growth of new blood vessels.

Corneal diseases affect the cornea and the conjunctiva. Cornea and conjunctiva form the outer surface of the eye, which is exposed to external environment, and are susceptible to infection agents, trauma, and/or exposure to chemicals, toxins, allergens etc. Cornea is also affected by autoimmune conditions, nutritional deficiencies and cancer. Corneal diseases may cause e.g. loss of vision, blurred vision, tearing, light sensitivity and pain. Diseases affecting cornea include, but are not limited to, keratitis, corneal dystrophy, corneal degeneration, Fuchs' dystrophy, cancer of cornea, and keratoconjunctivitis, Though surgical and medical treatment therapies for corneal diseases exist, in some cases, the diseases still remain severe and may cause blindness. There remains a need to efficient therapies for prevention, management and treatment of corneal diseases. Complement components of the cornea and the conjunctiva present in a normal eye include, but are not limited to, C1, C2, C3, C4, C5, C6, C7, Factor P (properdin) and factor B. Complement may have a role in corneal diseases, and antibodies targeting complement components of the eye may be used for prevention, treatment and/or management of corneal diseases.

Uveitis is an inflammation of the uvea, comprising the iris, choroids, and ciliary body. Early symptoms include eye redness, pain, irritation and blurred vision. Uveitis may lead to transient or permanent loss of vision. Uveitis may be associated with other diseases and conditions, such as infections, systemic diseases, non-infectious and autoimmune diseases. Complement components associated with an autoimmune form of uveitis include C3b and C4b. Uveitis may be managed or treated with vitrectomy, immunosuppressive drugs, corticosteroids or cytotoxic medication. However, despite the existing therapies, autoimmune uveitis is a serious condition and may lead to full or partial blindness. There remains a need for therapies for prevention, management, and treatment of uveitis targeting pathophysiology of the disease.

Retinopathy is a disease resulting from neovascularization (excessive growth of blood vessels) in the light-sensitive tissue of the eye, retina. Retinopathy may result in impaired vision or partial or full blindness. Retinopathy may be caused by systemic diseases, e.g. diabetes, or hypertension, trauma, excessive sun light exposure or ionizing radiation. Retinopathy is often treated with laser therapy. Medical treatments, such as antibodies, to control the growth of blood vessels, are also applied. However, despite the existing treatment methods, retinopathy is still a severe condition and may lead to blindness. Diabetic retinopathy is one of the leading causes of vision loss in middle-aged individuals. There remains a need for new therapies for prevention, management and/or treatment of retinopathy. For example, antibodies targeting blood vessel growth (e.g. vascular endothelial growth factor (′EGF), complement components (e.g. C3, C4, Clq, C9, C4b), and cluster of differentiation proteins (e.g. CD55, CD59) may be used for prevention, management and/or treatment of different retinopathies.

Photophobia is a condition referring to abnormal sensitivity or aversion to light. Photophobia is related to a number of ocular and nervous system diseases and disorders. Photophobia may be caused by damage to cornea or retina, albinism, overstimulation of the photoreceptors, excessive electric pulses to the central nervous system, or optic nerve. Photophobia may be associated with migraine, nervous system disorders (e.g. autism, dyslexia, encephalitis), infections (e.g. rabies, Lyme disease, mononucleosis), eye disorders (e.g. uveitis, corneal diseases, retinal diseases, scarring or trauma to cornea). As of today, there is no medical treatment for photophobia on the market. Photophobia is associated with calcitonin gene related peptide (CGRP) and CGRP receptors, and antibodies targeting CGRP may be used to prevent and/or treat photophobia, as described in U.S. Patent application US20120294802, the contents of which are herein incorporated by their reference.

In some embodiments, methods of the present disclosure may be used to treat subjects suffering from ocular diseases. In some cases, methods of the present disclosure may be used to treat subjects suspected of developing ocular diseases.

AAV Particles and methods of using the viral particles described in the present disclosure may be used to prevent, manage and/or treat psychiatric disorder. As a non-limiting example, the viral particles of the present disclosure comprise a nucleic acid sequence encoding at least one of the sequences described in Table 11.

### Systemic Diseases of the Blood, Heart and Bone

Systemic diseases are a category of conditions affecting the whole body, or many tissues and organs of the body. Systemic conditions associated with the blood, blood vessels, and heart, include, but are not limited to, heart failure, acute coronary syndrome, atherosclerosis, hypertension, lung disease, cardiomyopathy, hyperlipidemia, hypercholesterolemia, hypertriglyceridemia, blood clotting, cardiopulmonary bypass, myocardial infection, platelet aggregation and hemolytic diseases. In general, such conditions affect individual's quality of life and may be life-threatening. Cardiovascular diseases, referring to heart and blood vessels related conditions, are the leading cause of death worldwide. There remains a need for therapies affecting the pathophysiology of systemic heart, blood and blood circulation diseases. Antibodies for treating such conditions have been developed, targeting proteins such as, but not limited to, selectin P, integrin αIIbβ3, GPIIb/IIIa, RHD (Rh blood group, D antigen), PCSK9 (proprotein convertase subtilisin/kexin type 9), oxLDL (Oxidized low-density lipoprotein), CD20 (B-lymphocyte antigen), ANGPTL3 (Angiopoietin-tike 3), F9 (human factor 9), F10 (human factor 10), TFPI (Tissue Factor Pathway Inhibitor (Lipoprotein-Associated Coagulation inhibitor)), CD41 (Integrin, Alpha 2b (Platelet Glycoprotein lib Of IIb/IIIa Complex, Antigen CD41)).

In some embodiments, methods of the present disclosure may be used to treat subjects suffering from blood, blood circulation and heart related systemic diseases. In some cases, methods of the present disclosure may be used to treat subjects suspected of developing systemic blood, blood circulation and heart related systemic diseases.

Osteoporosis is a disease characterized by a reduced bone mineral density, and disrupted bone microarchitecture. Individuals with osteoporosis have a high susceptibility to bone fractures. Osteoporosis causes disability especially in the elderly, and may be fatal.

There are medical therapies for management of the osteoporosis, and other conditions associated with reduced bone density, such as calcitonin, bisphosphonates, estrogen replacement and selective estrogen modulators for prevention of bone loss, and anabolic agents to increase bone mass and bone mineral density. However, the present medical therapies have side effects and/or require frequent administration. There remains a need for efficient and long lasting medical therapy affecting the pathophysiology of osteoporosis and other conditions associated with reduced bone density, such as antibody therapies. Antibodies for treatment of osteoporosis are on the market, e.g. blosozumab (developed by Eli Lilly and Co.) targeting sclerostin (SOST) for increasing bone density, and denosumab (developed by Amgen) targeting TNF SF11 (Tumor Necrosis Factor (Ligand) Superfamily, Member 11) for treatment of bone loss.

In some embodiments, methods of the present disclosure may be used to treat subjects suffering from osteoporosis and/or other conditions associated with reduced bone density. In some cases, methods of the present disclosure may be used to treat subjects suspected of developing osteoporosis and/or other conditions associated with reduced bone density.

AAV Particles and methods of using the viral particles described in the present disclosure may be used to prevent, manage and/or treat systemic diseases of the blood, heart and/or bone. As a non-limiting example, the viral particles of the present disclosure comprise a nucleic acid sequence encoding at least one of the sequences described in Table 12.

### Immune System & Autoimmune Disease

Human immune system is a complex mechanism for identifying and removing harmful environmental agents and repairing the harm and damage caused by them. The basis of the immune system is ability to identify body's own substances from substances acquired. The immune response system can be divided into innate and adaptive systems. The innate system is present at all times and includes macrophages, dendritic cells, myeloid cells (neutrophils, mast cells, basophils, eosinophils) NK cells, complement factors and cytokines. The adaptive system responses to infectious agents, and include T and B lymphocytes, antibodies and cytokines. Activation of T and B cells in the absence of an infectious agents leads to autoimmune diseases (see, e.g. Mackay et al., 2001, N Engl J Med, Vol. 345, No. 5, and references therein). Autoimmune diseases may affect a number of body's tissues and functions, e.g. joints, skin, blood vessels, muscles, organs, intestine etc. Autoimmune diseases arise from and overactive and misguided immune response to body's natural tissues and species. Autoimmune diseases and conditions include, but are not limited to, rheumatoid arthritis, diabetes type 1, systemic lupus erythematosus, celiac sprue, psoriasis, Graves' disease, and Lyme disease. Autoimmune diseases may be caused by infections, drugs, environmental irritants, toxins, and/or genetic factors. Autoimmune diseases affect up to 50 million individuals in the US. Two most common autoimmune diseases are rheumatoid arthritis and autoimmune thyroiditis, together affecting approximately 5% of population in Western countries.

Though medical therapies for autoimmune diseases exits, the diseases may still significantly lower the quality of life, or even be fatal. There remains a need for medical therapies affecting the pathophysiology of autoimmune diseases. Autoimmune disease pathophysiology is associated with a number of factors and may be prevented and/or treated by antibodies targeting associated proteins. Such targets include, but are not limited to, infectious agents; environmental triggers (e.g. gliadin); targets affecting cytokinone production or signaling (e.g. TNFa (tumor necrosis factor alpha), IL-1 (interleukin 1-receptor), IL-2 (interleukin-.2), IL-2R (interleukin-2 receptor), IL-7 (interleukin-7), IL-10 (interleukin-10), IL-10R (interleukin-10 receptor), interferon-y, STAT-3 (Signal transducer and activator of transcription 3), STAT-4 (Signal transducer and activator of transcription 4), TGF beta (transforming growth factor beta), T cell trans TGF beta); T cell regulators (e.g. CTLA4 (Cytotoxic T-Lymphocyte-Associated Protein 4)); complement components (e.g. C1 and C4); TNFa (tumor necrosis factor alpha) and TNFb (tumor necrosis factor beta); T cell regulators (e.g. CD1); epitopes of B and T cells; and/or other targets, such as those associated with B and C cells. (see, e.g. Mackay et al., 2001, N Engl J Med, Vol. 345, No, 5, and references therein).

In some embodiments, methods of the present disclosure may be used to treat subjects suffering from an autoimmune disease. In some cases, methods of the present disclosure may be used to treat subjects suspected of developing an autoimmune disease.

AAV Particles and methods of using the viral particles described in the present disclosure may be used to prevent, manage and/or treat immune system and autoimmune disease. As a non-limiting example, the viral particles of the present disclosure comprise a nucleic acid sequence encoding at least one of the sequences described in Table 9.

### Inflammatory Disorders and Inflammation

Inflammation is a natural response of the body to an irritation e.g. by infection, damaged cells or other harmful agents. The purpose of the inflammation is to remove the cause of irritation and necrotic cells and damaged tissues and initiate cell and tissue repair. Inflammation has a role in majority of diseases. Inflammatory disorders are abnormalities in the body's ability to regulate inflammation. Over 100 disorders associated with high level of inflammation have been identified, including, but not limited to, Alzheimer's, ankylosing spondylitis, arthritis (osteoarthritis, rheumatoid arthritis (RA), psoriatic arthritis), asthma, atherosclerosis, Crohn's disease, colitis, dermatitis, diverticulitis, fibromyalgia, hepatitis, irritable bowel syndrome (IBS), systemic lupus erythematous (SLE), nephritis, Parkinson's disease, and ulcerative colitis. Many inflammatory disorders are severe, and even life-threatening. Antibodies targeting proteins associated with inflammation may be used to prevent, manage or treat inflammatory disorders as well as inflammation associated diseases.

A large number of proteins are associated in inflammation, including, but not limited to, TNF (anti-tumor necrosis factor), IL1R (Interleukin-1 receptor), IL-6R (Interleukin-6 receptor), Alpha integrin subunit, CTLA4 (Cytotoxic T-Lymphocyte-Associated Protein 4), and CD20 (see, e.g. Kotsovilis and Andreakos, 2014, Michael Steinitz (ed.), Human Monoclonal Antibodies: Methods and Protocols, Methods in Molecular Biology, vol. 1060, and references therein). For example, adalimumab (developed by Abbot Laboratories) is a TNF-targeting antibody for rheumatoid arthritis and other arthritises, psoriasis, and Crohn's disease and Natalizumab (developed by Biogen Idec) is an antibody targeting alpha 4-integring for treatment of Crohn's disease. Additionally, plethora of cytokines, chemokines, adhesion and co-stimulatory molecules, receptors, as well as diverse cell types, may have a role in inflammatory diseases.

In some embodiments, methods of the present disclosure may be used to treat subjects suffering from an inflammatory disease. In some cases, methods of the present disclosure may be used to treat subjects suspected of developing an inflammatory disease.

AAV Particles and methods of using the viral particles described in the present disclosure may be used to prevent, manage and/or treat inflammatory disorders and inflammation. As a non-limiting example, the viral particles of the present disclosure comprise a nucleic acid sequence encoding at least one of the sequences described in Table 9.

### Other Therapeutic Targets

The viral particles or pharmaceutical compositions of the present disclosure useful in preventing or treating disease may alternatively, or in combination, encode an antibody that binds a target antigen including, but not limited to, any of the following, including fragments or variants thereof, α-synuclein (monomers, oligomers, aggregates, fragments), ABCA1 (ATP-binding cassette, sub-family A, member 1), ABCA4 (ATP-binding cassette, sub-family A, member 4), ABCB1 (ATP-binding cassette, sub-family B, member 1), ACE (angiotensin I converting enzyme), ACKR1 (atypical chemokine receptor 1 (Duffy blood group)), AMPA (DL-a-amino-3-hydroxy-5-methyl-4-isoxazole propionic acid), ACTH (Adrenocorticotropic Hormone), ACVR2A (Activin receptor type-2A), ACVR2B (Activin receptor type-2B), ADDL (Adducin-Like Protein 70), ADORA2A (adenosine A.2a. receptor), ADRA2A (adrenoceptor alpha 2A), AIFM1 (apoptosis-inducing factor), AKT1 (RAC-alpha serine/threonine-protein kinase), ALK-1 (activin receptor-like kinase 1), Alpha beta fibril, alpha subunit (basic helix-loop-helix transcription factor), AMT (Aminomethyltransferase), Amyloid 13 (monomers, oligomers, aggregates, fragments), amyloid or amyloid-like proteins, ANGPTL3 (Angi opoietin-Like 3), ANGTP1 (angiopoitin 1), ANGTP2 (angiopoietin 2), ANK3 (ankyrin 3), ANKG (ankyrin G), Annexin IV, phospholipid, Anx-A1 (annexin A1), APOE (apolipoprotein E), APP (amyloid beta precursor protein), ARSD (Aryl sulfatase D), ATM (Ataxia Telangiectasia Mutated serine/threonine kinase), ATXN1 (ataxin 1), ATXN2 (ataxin 2), ATXN3 (ataxin 3), ATXN7 (ataxin 7), B Lymphocyte Stimulator, BDNF (brain-derived neurotrophic factor), beta A4 peptide/Alpha beta 4, beta A4 peptide, Alpha beta 5, bAlpha beta 6, Alpha beta 7, Alpha beta 8, Alpha beta 9, Beta-secretases (RACE), BRAE (B-Raf Proto-Oncogene, Serineffhreonine Kinase), Properdin (factor P), Factors Ba and Bb, C1, Clq (complement component 1, subcomponent q), C2, C3, C4, C3a, C3b, C5, C5a, C5b, C6, C7, C8, C9 and C5b-9 (complement components), CAIX (Carbonic anhydrase IX) CA 125 (cancer antigen 125), CACNA1A (calcium channel voltage-dependent P/Q type alpha 1A subunit), cadherins, CA-IX (carbonic anhydrase 9), CALCA (calcitonin-related polypeptide alpha), CCKBR (cholecystokinin B receptor), CCL1.1 (eotaxin-1), CCL2 (Chemokine (C-C Motif) Ligand 2), CD11 (integrin alpha component), CD147 (basigin), CD154 (CD40L), CD19 (Cluster of Differentiation 19), CD2 (cluster of differentiation 2), CD20 (B-lymphocyte antigen), CD200 (cluster of differentiation 200), CD22 (cluster of differentiation 22), CD221 (insulin-like growth factor 1 (IGF-1) receptor), CD248 (Endosialin), CD26 (Dipeptidyl peptidase-4), CD27 (antigen precursor), CD274 (cluster of differentiation 274), CD28 (Cluster of Differentiation 28), CD29 (Integrin, Beta 1), CD3 (cluster of differentiation 3), CD30 (cluster of differentiation 30), CD31 (cluster of differentiation 31), CD33 (cluster of differentiation 33), CD37 (Leukocyte antigen), CD38 (cyclic ADP ribose hydrolase), CD3E (T-Cell Surface Antigen T3/Leu-4 Epsilon Chain), CD4 (T-Cell Surface Antigen T4/Leu-3), CD40 (CD40 Molecule, INF Receptor Superfamily Member 5), CD41 (Integrin, Alpha 2b (Platelet Glycoprotein fib Of IIb/IIIa Complex, Antigen CD41)), CD44 (cluster of differentiation 44), CD51 (integrin alpha 1), CD52 (Human Epididymis-Specific Protein 5), CD55 (Decay Accelerating Factor For Complement (Cromer Blood Group)), CD58 (lymphocyte function-associated antigen 3), CD59 (MAC-inhibitory protein), CD6 (cluster of differentiation 6), CD70 (cluster of differentiation 70, ligand for CD27), CD74 (HLA class II histocompatibility antigen gamma chain), CD79B (immunoglobulin-associated beta), CEA (Carcinoembryonic antigen), CFHR1 (Complement Factor H-Related 1), CGRP (Calcitonin gene-related peptide), CHMP213 (charged multivesicular body protein 2B), CHRNA4 (cholinergic receptor nicotinic alpha 4 (neuronal)), CHRNB2 (cholinergic receptor nicotinic beta 2 (neuronal)), CISD2 (CDGSH iron sulfur domain 2), CLEC16A (C-type lectin domain family 16 member A), CLRN1 (clarin 1), CNR1 (cannabinoid receptor 1), CNTNAP2 (contactin associated protein-like 2), COMT (catechol-O-methyltransferase), CRB1 (crumbs family member 1, photoreceptor morphogenesis associated), CRX (cone-rod homeobox), CRY (crystallin), CSF1R (Colony Stimulating Factor 1 Receptor), CSF2 (Colony Stimulating Factor 2 (Granulocyte-Macrophage)), CSF2RA (Colony Stimulating Factor 2 Receptor, Alpha, Low-Affinity), CTGF (Connective Tissue Growth Factor), CTLA4 (Cytotoxic T-Lymphocyte-Associated Protein 4), CXC (chemokine receptor type 4), CXCL10 (Chemokine (C-X-C Motif) Ligand 10), DDC (dopa decarboxylase (aromatic L-amino acid decarboxylase)), DIABLO (LAP-Binding Mitochondrial Protein), differentiation factor 8 (GDF8), DISC1 (disrupted in schizophrenia 1), DLL3 (Delta-Like 3 (Drosophila)), DLL4 (Delta-Like 4 (Drosophila)) DPP4 (dipeptyl-peptidase 4), DPP6 (dipeptidyl-peptidase 6), DR6 (Death receptor 6), DRD1 (dopamine receptor D1), DRD2 (dopamine receptor D2), DRD4 (dopamine receptor D4), DRD5 (dopamine receptor 5), DRD5 (dopamine receptor D5), DTNBP1 (dystrobrevin binding protein 1), EAG1 (Ether-A-Go-Go Potassium Channel 1), EDB (fibronectin extra domain-B), EDNRA (endothelin receptor type A), EFNA1 (Ephrin-A1) EGET; (EGF-Like-Domain, Multiple 7), EGFR/ERBB1/HER1 (epidermal growth factor receptor 1), EN2 (Engrailed Homeobox 2), EPCAM (Epithelial cell adhesion molecule), EPHA3 (EPH Receptor A3), episialin (a carcinoma-associated mucin, MUC-1), ERBB2 (epidermal growth factor receptor 2), ERBB3 (epidermal growth factor receptor 3), ESR1 (estrogen receptor 1), F3 (coagulation factor Hp, F9 (human factor 9), F10 (human factor 10), FAAH (fatty acid amide hydrolase), Factor D C3 proactivator convertase), humanized IgG1, humanized IgG2, FAP (Fibroblast Activation Protein, Alpha), FBN2 (fibrillin 2), FBP (Folate-binding protein), FcγRIIB (Fc receptor gamma B), FcγRIIIA (Fc receptor gamma A), FLT1 (Fms-Related Tyrosine Kinase 1), FOLR1 (folate receptor alpha), Frizzled receptor, FAN (frataxin), FUS/TLS (RNA binding protein), G protein-coupled, GAA (glucosidase alpha acid), Gc-globulin (Vitamin D binding protein), Gangliosides, GD2 (ganglioside G2), GD3 (ganglioside g3), GM2 (monosialotetrahexosylganglioside 2) (GDF-8 (myostatin), GDNF (glial cell derived neurotrophic factor), GDNF (glial cell derived neurotrophic factor), GFAP (glial fibrillary acidic protein), GFRα3 (GDNF family receptor alpha-3), ghrelin, GIT1 (G protein-coupled receptor kinase interacting ArfGAP 1), GJA (Gap junction protein), GLDC Glycine Dehydrogenase (Decarboxylating), glycoprotein NMB (GPM/1B), gpA33 (Glycoprotein A33 (Transmembrane)), GPC3 (glypican 3), GRIN2B (glutamate receptor ionotropic N-methyl D-aspartate 2B), GRN (granulin), GDF8 (growth differentiation factor 8), GTPases (guanosine triphosphate), GSTP1 (glutathione S-transferase pi 1), GUCA1A (guanylate cyclase activator 1A (retina), GUCY2C (anti-GCC), HMCN1 (hemicentin 1), HGF (Hepatocyte Growth Factor), HIF1A (hypoxia inducible factor 1, HINT1 (histidine triad nucleotide binding protein 1), HIST3H3 (Histone 113), histone, HLA-DQB1 (major histocompatibility complex class II DQ beta 1), HLA-DR (MHC class cell surface receptor), HLA-DRB1 (major histocompatibility complex class DR beta 1), hNav1.7 (sodium ion channel), HTR1A (5-hydroxytryptamine (serotonin) receptor 1A G protein-coupled), HTR2A (5-hydroxytryptamine (serotonin) receptor 2A, HTR2A (5-hydroxytryptamine (serotonin) receptor 2A G protein-coupled), (huntingtin), IAP-binding mitochondrial protein, IFNAR1 (Interferon (Alpha, Beta And Omega) Receptor 1), IFNB1 (interferon beta 1 fibroblast), IFN-γ (Interferon gamma), IGF-1 receptor, IGF1R (insulin-like growth factor 1 receptor), IGF-I (insulin-like growth factor 1), IGG1 (immunoglobulin subclass 1), IgG2 (immunoglobulin subclass 2), IgG4 (immunoglobulin subclass 4), KM1H (Immunoglobulin Heavy Constant Epsilon) IL 1B (interleukin 1 beta), IL12 (interleukin 12), IL12B (interleukin 12B), IL13 (interleukin 13), 11.L17A (interleukin 17A), IL17F (interleukin 17F), ILIA (interleukin 1A), IL1B (interleukin 1 beta), IL1-Ri (Interleukin 1 receptor, type I), 1120 (Interleukin 20), IL23A (interleukin 23A), IL-23p19 subunit (interleukin 23 subunit p19), IL2RA (interleukin 2 receptor alpha), IL4R (interleukin 4 receptor alpha, IL6 (interleukin 6), IL6R (interleukin 6 receptor), IL7R (interleukin 7 receptor), ILGF2 (insulin like growth factor 2), INS (insulin), Integrin a5(31, Integrin αVβ3, integrin αIIbβ3/GPIIb/IIIa, IP6K2 (inositol hexakisphosphate kinase 2), ITGA4 (Integrin, Alpha 4 (Antigen CD49D, Alpha 4 Subunit Of VLA-4 Receptor)), ITGB7 (Integrin, Alpha 7 (Antigen CD49D, Alpha 4 Subunit Of VLA-7 Receptor)), ITGAL (integrin alpha L chain), ITGAV ((Vitronectin Receptor, Alpha Polypeptide, Antigen CD51), ITGB3 (Integrin alpha-V/beta-3), KCNQ2 (potassium channel voltage gated KQT-like subfamily Q member 2), KDR (Kinase Insert Domain Receptor), KIR2D (killer immunoglobulin-like receptor (KIR) 2D subtype), KLRC1 (Killer Cell Lectin-Like Receptor Subfamily C, Member 1), LAG-3 (Lymphocyte-activation gene 3), Le (y) (Lewis y) antigen, LINGO (Leucine rich repeat and Immunoglobin-like domain-containing protein 1), LOXL2 (Lysyl oxidase homolog 2), LPG (lysophosphatidylglucoside), LPS (Lipopolysaccharides), LRP1 (low density lipoprotein receptor-related protein 1), LRRC6 (Leucine Rich Repeat Containing 6), LRRK2 (leucine-rich repeat kinase 2), LTA (Lymphotoxin Alpha), MAF (maf avian musculoaponeurotic fibrosarcoma oncogene homolog), MAG (Myelin Associated Glycoprotein), MAI (myelin associated inhibitor), MAOB (monoamine oxidase B), MAPT (microtubule-associated protein tau), MBP (myelin basic protein), MCAT (monocyte chemotactic and activating factor), MCP-1 (Monocyte chemoattractant protein-i), MBL (mannose binding lectin), mannose, MET (Tyrosine-Protein Kinase Met), MIF (Macrophage Migration Inhibitory Factor (Glycosylation-Inhibiting Factor), MS4A1 (Membrane-Spanning 4-Domains, Subfamily A, Member 1), MSLN (Mesothelin), MST1R (Macrophage Stimulating 1 Receptor), MSTN (myostatin), MUC1/Episialin MUC5AC (Mucin 5AC, Oligomeric Mucus/Gel-Forming), mucin CanAg (glycoform. MUC-1), Mucins, myostatin, myostatin antagonists, N-acetyl glucosamine, NCAM1 (Neural Cell Adhesion Molecule 1) Neu5Gc/NGNA (Neurogenin A), neuregulin (NRG), neurokinin B, NGF (Nerve growth factor), NMDA (N-methyl-D-aspartate), NOGO (Neurite outgrowth inhibitor), NOGO receptor-i, Nogo-66, NOGOA/NiG (Neurite Outgrowth Inhibitory Fragments of NOGOA), Notch receptor, NOTCH-1 (Notch homolog 1, translocation-associated (Drosophila)), NRG1 (neuregulin 1), INRP1 (Neuropilin 1), NT-3 trkC ligand, N-terminal region of Aβ8-x peptide, OGG1 (8-oxoguanine DNA glycosylase), oligomers of N-terminal truncated Aβ, OPA2 (Optic Atrophy 2), OPA3 (Optic Atrophy 3), oxLDL (Oxidized low-density lipoprotein), P75 (Low-affinity Nerve Growth Factor Receptor), PAND1 9Panic disorder 1), PAND2 (Panic disorder 2), PAND3 9 Panic disorder 3), PARK2 (parkin RBR E3 ubiquitin protein ligase), PCSK9 (proprotein convertase subtilisin/kexin type 9), PD-1 (Programmed cell death protein 1), PD-2 (Programmed cell death protein 2), PD-3 (Programmed cell death protein 3), PD-4 (Programmed cell death protein 4), PD-S(Programmed cell death protein 5), PD-6 (Programmed cell death protein 6), PD-7 (Programmed cell death protein 7), PD-8 (Programmed cell death protein 8), PDGFRA (Platelet-derived growth factor receptor alpha), PDGFRB (Platelet-derived growth factor receptor beta), PD-L1 (Programmed cell death protein 1 ligand), PEX7 ((Peroxisomal Biogenesis Factor 7), PHOBS (phobia specific), PhosphatidyL-serine, chimeric IgG1, Phosphatide L-serine, Chimeric IgG2, PINK1 (PTEN induced putative kinase 1), platelet-derived growth factor receptor beta PDGFRB, PLAU (plasminogen activator urokinase), PLP (protelopid protein), PMP22 (peripheral myelin protein 22), POLG (polymerase (DNA directed) gamma), PRDM16 (PR domain containing 16), Prion proteins, PrP, PrPC, PrPSc, PRKCG (protein kinase C gamma), PSEN1 (presenilin 1), PSEN2 (presenilin 2), PSMA (Prostate-specific membrane antigen), PTGS2 (prostaglandin-endoperoxide synthase 2 (prostaglandin G/H synthase and cyclooxygenase)), PIPN11 (Tyrosine-protein phosphatase non-receptor type 11), PVRL4 (Poliovirus Receptor-Related 4), PVRL5 (Poliovirus Receptor-Related 5), pyroglutamated A (3, RAfl (proto-oncogene serine/threonine-protein kinase), RAGE protein, RANKL (Receptor activator of nuclear factor kappa-B ligand), RCAN1 (regulator of calcineurin 1), RDh12 (retinol dehydrogenase 12 (ail-trans/9-cis/11-cis)), RGI A (Repulsive guidance molecule A), RHD (Rh blood group, D antigen), RHO (rhodopsin), RPE65 (retinal pigment epithelium-specific protein 65 kDa), RTN4 (Reticulon-4, NOGO), S100B (calcium-binding protein B), S 1P4 (Type 4 sphingosine 1-phosphate G protein-coupled receptor), SCN1A (Sodium Channel, Voltage Gated, Type I Alpha Subunit), SDC1 (Syndecan 1), selectin P, SHANK3 (S113 And Multiple Ankyrin Repeat Domains 3), SLAMF7 (SLAM Family Member 7), SLC18A2 (solute carrier family 18 (vesicular monoamine transporter, member 2), SLC1A.2 (solute carrier family 1 (glial high affinity glutamate transporter, member 2), SLC34A2 (Solute Carrier Family 34 (Type II Sodium/Phosphate Cotransporter), SLC6A3 (solute carrier family 6 (neurotransmitter transporter) member 3), SLC6A4 (Solute Carrier Family 6 (Neurotransmitter Transporter), SMN1 (survival of motor neuron 1 telomeric), SMN2 (survival of motor neuron 2 centromeric), SNCA (synuclein alpha (non A4 component of amyloid precursor)), SNCA. (synuclein alpha (non A4 component of amyloid precursor), SNCB (synuclein beta), SOD1 (superoxide dismutase 1 soluble), SOST (Sclerostin), sphingosine-1-phosphate, SQSTM1 (sequestosome 1), STEAP1 (Six Transmembrane Epithelial Antigen Of The Prostate 1), SLIF2 (Sulfatase 2), TACR1 (tachykinin receptor 1), TAG-72 (Tumor-associated glycoprotein 72), TARDBP (TAR DNA binding protein), tau antigen, tau protein, tau pS422, TDP-43, tenascin, tenascin C, TFPI (Tissue Factor Pathway inhibitor (Lipoprotein-Associated Coagulation Inhibitor)), TGF beta (Transforming growth factor beta), TH (Tyrosine hydroxylase), TkrC (Tropomyosin receptor kinase C), TMEFF2 (Transmembrane Protein With EGF-Like And Two Follistatin-Like Domains 2), TMEFF3 (Transmembrane Protein With EGF-Like And Two Follistatin-Like Domains 3), TNF (tumor necrosis factor), TNFa (tumor necrosis factor alpha), TNTRSF1OB (Tumor Necrosis Factor Receptor Superfamily, Member 10b), TNFRSF12A (Tumor Necrosis Factor Receptor Superfamily, Member 12A), TNFRSF8 (Tumor Necrosis Factor Receptor Superfamily, Member 8), TNFRSF9 (Tumor Necrosis Factor Receptor Superfamily, Member 9), TNF SF11 (Tumor Necrosis Factor Receptor Superfamily, Member 11), TNFSF13B (Tumor Necrosis Factor Receptor Superfamily, Member 13b), TNF-α, (Tumor Necrosis Factor alpha)), TNNT2 (troponin T type 2), TOR1A (torsin family 1 member A (torsin A)), TPBG (Trophoblast Glycoprotein), TPH2 (tryptophan hydroxylase 2), TRAILR1 (Death receptor 4), TRAILR2 (Death receptor 5), TrkA (Tropomyosin receptor kinase A), TRPV4 (Transient Receptor Potential Cation Channel, Subfamily V, Member 4), TSC2 (tuberous sclerosis 2), TULP1 (tubby like protein 1), tumor necrosis factor related protein 5, tumor specific glycosylation of MUC1, tumor-associated calcium signal transducer 2, tumor protein pS3, 71YRP1 (glycoprotein 75), UCHl1 (ubiquitin carboxyl-terminal esterase L1 (ubiquitin thiolesterase)), UNC-13A (unc-13 homolog A), USH1C (Usher Syndrome 1C), USI-12A (Usher Syndrome 2A (Autosomal Recessive, Mild), VEGF (Vascular endothelial growth factor), VEGF A (Vascular endothelial growth factor A), C5, Factor P, Factor D, EPO (Erythropoietin), EPOR (EPO receptor), Interleukins, IL-113, IL-17A, IL-10, TNFa, FGFR2 (Fibroblast Growth Factor Receptor 2), VEGFR (vascular endothelial growth factor receptor), VEGFR2 (vascular endothelial growth factor receptor 2), vimentin, voltage gated ion channels, VWF (Von Willebrand Factor), WFS1 (Wolfram syndrome 1 (wolframin)), YES1 (Yamaguchi Sarcoma Viral Oncogene Bornolog 1).

In some embodiments, the viral particle of the present disclosure, useful in treating a non-infectious disease, targets an antigen considered to be useful in the treatment of a different disease. As a non-limiting example, a viral particle or pharmaceutical composition thereof used for the treatment of cancer, immune system dysfunctions or inflammatory disease may likewise be used for the treatment of a neurodegenerative disorder such as, but not limited to, A1), PD, HD, ALS, SMA, or DLB.

AAV Particles and methods of using the viral particles described in the present disclosure may be used to prevent, manage and/or treat non-infectious disease. As a non-limiting example, the viral particles of the present disclosure comprise a nucleic acid sequence encoding at least one of the sequences described in Table 3-12.

### Therapeutic Applications: Tau

The present disclosure additionally provides a method for treating neurological diseases and/or disorders in a mammalian subject, including a human subject, comprising administering to the subject any of the viral particles of the disclosure. In some cases, neurological diseases and/or disorders treated according to methods described herein include indications involving irregular expression or aggregation of tau. Such indications may include, but are not limited to Alzheimer's disease (AD), frontotemporal dementia and parkinsonism linked to chromosome 17 (FTDP-17), Frontotemporal lobar degeneration (FTLD), chronic traumatic encephalopathy (CTE), Progressive Supranuclear Palsy (PSI)), Down's syndrome, Pick's disease, Corticobasal degeneration (CBD), Amyotrophic lateral sclerosis (ALS), Prion diseases, Creutzfeldt-Jakob disease (CID), Multiple system atrophy, Tangle-only dementia, and Progressive subcortical gliosis.

In some embodiments, methods of treating neurological diseases and/or disorders in a subject in need thereof may comprise the steps of: (1) deriving, generating and/or selecting an anti-tau antibody, antibody-based composition or fragment thereof; (2) producing a viral particle with a viral genome that includes a payload region encoding the selected antibody of (1); and (3) administering the viral particle (or pharmaceutical composition thereof) to the subject.

The present disclosure provides a method for administering to a subject in need thereof, including a human subject, a therapeutically effective amount of the viral particles of the disclosure to slow, stop or reverse disease progression. As a non-limiting example, disease progression may be measured by cognitive tests such as, but not limited to, the Mini-Mental State Exam (MMSE) or other similar diagnostic tool(s), known to those skilled in the art. As another non-limiting example, disease progression may be measured by change in the pathological features of the brain, CSF or other tissues of the subject, such as, but not limited to a decrease in levels of tau (either soluble or insoluble). In some embodiments levels of insoluble hyperphosphorylated tau are decreased. In some embodiments levels of soluble tau are decreased. In some embodiments both soluble and insoluble tau are decreased. In some embodiments, levels of insoluble hypetphosphorylated tau are increased. In some embodiments levels of soluble tau are increased. In some embodiments both insoluble and soluble tau levels are increased. In some embodiments, neurofibrillary tangles are decreased in size, number, density, or combination thereof. In another embodiment, neurofibrillary tangles are increased in size, number, density or combination thereof.

### Alzheimer's Disease

Alzheimer Disease (AD) is a debilitating neurodegenerative disease currently afflicting more than 35 million people worldwide, with that number expected to double in coming decades. Symptomatic treatments have been available for many years but these treatments do not address the underlying pathophysiology. Recent clinical trials using these and other treatments have largely failed and, to date, no known cure has been identified.

The AD brain is characterized by the presence of two forms of pathological aggregates, the extracellular plaques composed of β-amyloid (Aβ) and the intracellular neurofibrillary tangles (NFT) comprised of hyperphosphorylated microtubule associated protein tau. Based on early genetic findings, β-amyloid alterations were thought to initiate disease, with changes in tau considered downstream. Thus, most clinical trials have been Aβ-centric. Although no mutations of the tau gene have been linked to AD, such alterations have been shown to result in a family of dementias known as tauopathies, demonstrating that changes in tau can contribute to neurodegenerative processes. Tau is normally a very soluble protein known to associate with microtubules based on the extent of its phosphorylation. Hyperphosphorylation of tau depresses its binding to microtubules and microtubule assembly activity. In tauopathies, the tau becomes hyperphosphorylated, misfolds and aggregates as NFT of paired helical filaments (PHF), twisted ribbons or straight filaments. In AD, NFT pathology, rather than plaque pathology, correlates more closely with neuropathological markers such as neuronal loss, synaptic deficits, severity of disease and cognitive decline. NFT pathology marches through the brain in a stereotyped manner and animal studies suggest a trans-cellular propagation mechanism along neuronal connections.

Several approaches have been proposed for therapeutically interfering with progression of tau pathology and preventing the subsequent molecular and cellular consequences. Given that NFT are composed of a hyperphosphorylated, misfolded and aggregated form of tau, interference at each of these stages has yielded the most avidly pursued set of targets. Introducing agents that limit phosphorylation, block misfolding or prevent aggregation have all generated promising results. Passive and active immunization with late stage anti-phospho-tau antibodies in mouse models have led to dramatic decreases in tau aggregation and improvements in cognitive parameters. It has also been suggested that introduction of anti-tau antibodies can prevent the trans-neuronal spread of tau pathology.

The vectored antibody delivery (VAD) of tau disease associated antibodies of the present disclosure may be used to treat subjects suffering from AD and other tauopathies. In some cases, methods of the present disclosure may be used to treat subjects suspected of developing AD or other tauopathies.

### Frontotemporal Dementia and Parkinsonism Linked to Chromosome 17 (FTDP-17)

Although Alzheimer's disease is, in part, characterized by the presence of tau pathology, no known mutations in the tau gene have been causally linked to the disease. Mutations in the tau gene have been shown to lead to an autosomal dominantly inherited tauopathy known as frontotemporal dementia and parkinsonism linked to chromosome 17 (FTDP-17) and demonstrate that alterations in tau can lead to neurodegenerative changes in the brain. Mutations in the tau gene that lead to FTDP-17 are thought to influence splicing patterns, thereby leading to an elevated proportion of tau with four microtubule binding domains (rather than three). These molecules are considered to be more amyloidogenic, meaning they are more likely to become hyperphosphorylated and more likely to aggregate into NFT (Hutton, M. et al., 1998, Nature 393(6686):702-5). Although physically and behaviorally, FTDP-17 patients can appear quite similar to Alzheimer's disease patients, at autopsy FTDP-17 brains lack the prominent Aβ plaque pathology of an AD brain (Gotz, J. et al., 2012, British Journal of Pharmacology 165(5):1246-59). Therapeutically targeting the aggregates of tau protein may ameliorate and prevent degenerative changes in the brain and potentially lead to improved cognitive ability.

As of today, there is no treatment to prevent, slow the progression, or cure FTD. Medication may be prescribed to reduce aggressive, agitated or dangerous behavior. There remains a need for therapy affecting the underlying pathophysiology, such as antibody therapies targeting tau protein.

In some embodiments, the vectored antibody delivery of the present disclosure may be used to treat subjects suffering from FTDP-17. In some cases, methods of the present disclosure may be used to treat subjects suspected of developing FTDP-17.

### Chronic Traumatic Encephalopathy

Unlike the genetically linked tauopathies, chronic traumatic encephalopathy is a degenerative tauopathy linked to repeated head injuries. The disease was first described in boxers whom behaved “punch drunk” and has since been identified primarily in athletes that play American football, ice hockey, wrestling and other contact sports. The brains of those suffering from CTE are characterized by distinctive patterns of brain atrophy accompanied by accumulation of hyperphosphorylated species of aggregated tau in NFT. In CTE, pathological changes in tau are accompanied by a number of other pathobiological processes, such as inflammation (Daneshvar, D. H. et al., 2015 Mol Cell Neurosci 66(Pt B): 81-90). Targeting the tau aggregates may provide reprieve from the progression of the disease and may allow cognitive improvement.

As of today, there is no medical therapy to treat or cure CTE. The condition is only diagnosed after death, due to lack of in vivo techniques to identify CTE specific biomarkers. There remains a need for therapy affecting the underlying pathophysiology, such as antibody therapies targeting tau protein.

In some embodiments, the vectored antibody delivery methods of the present disclosure may be used to treat subjects suffering from CTE. In some cases, methods of the present disclosure may be used to treat subjects suspected of developing CTE.

### Prion Diseases

Prion diseases, also known as transmissible spongiform encephalopathies (TSEs), are a group of rare progressive conditions affecting the nervous system. The related conditions are rare and are typically caused by mutations in the PRNP gene which enables production of the prion protein. Gene mutations lead to an abnormally structured prion protein. Alternatively, the abnormal prion may be acquired by exposure from an outside source, e.g. by consumption of beef products containing the abnormal pion protein. Abnormal prions are misfolded, causing the brain tissue to degenerate rapidly. Prion diseases include, but are not limited to, Creutzfeldt-Jakob disease (CJD), Gerstmann-Sträussler-Scheinker syndrome (GSS), fatal insomnia (FFI), variably protease-sensitive prionopathy (VPSPr), and kuru. Prion diseases are rare. Approximately 350 cases of prion diseases are diagnosed in the US annually.

OD is a degenerative brain disorder characterized by problems with muscular coordination, personality changes including mental impairment, impaired vision, involuntary muscle jerks, weakness and eventually coma. The most common categories of CJD are sporadic, hereditary due to a genetic mutation, and acquired. Sporadic OD is the most common form affecting people with no known risk factors for the disease. The acquired form of CII) is transmitted by exposure of the brain and nervous system tissue to the prion. As an example, variant CJD (vCDJ) is linked to a bovine spongiform encephalopathy (BSE), also known as a ‘mad cow’ disease. CJD is fatal and patients typically die within one year of diagnosis.

Priori diseases are associated with an infectious agent consisting of an alternative conformational isoform of the prion protein, PrPSc. PrPSc replication is considered to occur through an induction of the infectious prion in the normal prion protein (PrPC). The replication occurs without a nucleic acid.

As of today, there is no therapy to manage or cure CJD, or other prion diseases. Typically, treatment is aimed at alleviating symptoms and increasing comfortability of the patient, e.g. with pain relievers. There remains a need for therapy affecting the underlying pathophysiology, such as antibody therapies targeting the priori protein.

In some embodiments, vectored antibody delivery methods of the present disclosure may be used to treat subjects suffering from a prion disease. In some cases, methods of the present disclosure may be used to treat subjects suspected of developing a prion disease.

### Neurodegeneration and Stroke

Neurodegenerative diseases and other diseases of the nervous system share many common features. Neurodegenerative diseases, in particular, are a group of conditions characterized by progressive loss of neuronal structure and function, ultimately leading to neuronal cell death. Neurons are the building blocks of the nervous system(s) and are generally not able to reproduce and/or be replaced, and therefore neuron damage and/or death is especially devastating. Other, non-degenerating diseases that lead to neuronal cell loss, such as stroke, have similarly debilitating outcomes. Targeting molecules that contribute to the deteriorating cell structure or function may prove beneficial generally for treatment of nervous system diseases, neurodegenerative disease and/or stroke.

Certain molecules are believed to have inhibitory effects on neurite outgrowth, contributing to the limited ability of the central nervous system to repair. Such molecules include, but are not limited to, myelin associated proteins, such as, but not limited to, RGM (Repulsive guidance molecule), NOGO (Neurite outgrowth inhibitor), NOGO receptor, MAG (myelin associated glycoprotein), and MAI (myelin associated inhibitor). In some embodiments, the vectored antibody delivery of the present disclosure is utilized to target the aforementioned antigens (e.g., neurite outgrowth inhibitors).

Many neurodegenerative diseases are associated with aggregation of misfolded proteins, including, but not limited to, alpha synuclein, tau, amyloid β, prion proteins, TDP-43, and huntingtin (see, e.g. De Genst et al., 2014, Biochim Biophys Acta; 1844(11):1907-1919, and Yu et al., 2013, Neurotherapeutics.; 10(3): 459-472, references therein). The aggregation results from disease-specific conversion of soluble proteins to an insoluble, highly ordered fibrillary deposit. This conversion is thought to prevent the proper disposal or degradation of the misfolded protein, thereby leading to further aggregation. Conditions associated with alpha synuclein and tau may be referred to as “synucleinopathies” and “tauopathies”, respectively. In some embodiments, the vectored antibody delivery of the present disclosure is utilized to target the aforementioned antigens (e.g., misfolded or aggregated proteins).

AAV Particles and methods of using the viral particles described in the present disclosure may be used to prevent, manage and/or treat tauopathies or tau associated disease. As a non-limiting example, the viral particles of the present disclosure comprise a nucleic acid sequence encoding at least one of the sequences described in Table 13.

### Therapeutic Applications: Infectious Diseases

Viral particles and methods of using the viral particles described in the present disclosure may be used to prevent, manage and/or treat infectious disease. As a non-limiting example, the viral particles of the present disclosure comprise a nucleic acid sequence encoding at least one of the sequences described in Tables 17, and 32-53.

The methods, components and compositions of the present disclosure may be used to diagnose, prevent, treat and/or manage infectious diseases. Infectious diseases, also known as transmissible diseases or communicable diseases, are caused by invasion and multiplication of agents in the body. infection agents are species typically not present within the body and may be, but are not limited to, viruses, bacteria, prions, nematodes, fungus, parasites or arthropods. Additionally, an infection or symptoms associated with an infection may be caused by one or more toxins produced by such agents. Humans, and other mammals, react to infections with an innate immune system response, often involving an inflammation. The illnesses and symptoms involved with infections vary according to the infectious agent. Many infections may be subclinical without presenting any definite or observable symptoms, whereas some infections cause severe symptoms, require hospitalization or may be life-threatening. Some infections are localized, whereas some may overcome the body through blood circulation or lymphatic vessels. Some infections have long-term effects on wellbeing of infected individuals.

Infectious agents may be transmitted to humans via different routes. For example, infection agents may be transmitted by direct contact with an infected human, an infected animal, or an infected surface. Infections may be transmitted by direct contact with bodily fluids of an infected human or an animal, e.g. blood, saliva, sweat, tears, mucus, female ejaculate, semen, vomit or urine. For example, infection may be transmitted by a fecal-oral route, referring to an infected person shedding the virus in fecal particles which then enters to person's mouth causing infection. The fecal-oral route is especially common transmission route in environments with poor sanitation and hygiene. Non-limiting examples of agents transmitted by the fecal-oral route include bacteria, e.g. shigella, Salmonella typhi and Vibrio Cholerae, virus, e.g. norovirus, rotavirus, enteroviruses, and hepatitis A, fungi, e.g. Entamadeba histolytica, parasites, tape worms, transmitted by contaminated food or beverage, leading to food poisoning or gastroenteritis. Infections may be transmitted by a respiratory route, referring to agents that are spread through the air. Typical examples include agents spread as small droplets of liquid or as aerosols, e.g. respiratory droplets expelled from the mouth and nose while coughing and sneezing. Typical examples of respiratory transmitted diseases include the common cold mostly implicated to rhinoviruses, influenza caused by influenza viruses, respiratory tract infections caused by e.g. respiratory syncytial virus (RSV). Infections may be transmitted by a sexual transmission route. Examples of common sexually transmitted infections include e.g, human immunodeficiency virus (HIV) causing acquired immune deficiency syndrome (AIDS), chlamydia caused by Neisseria gonorrhoeae bacteria, fungal infection Candidiasis caused by Candida yeast, and Herpes Simplex disease caused by herpes simplex virus. Infections may be transmitted by an oral transmission route, e.g. by kissing or sharing a drinking glass. A common infection transmitted by oral transmission is an infectious mononucleosis caused by Epstein-Barr virus. Infections may be transmitted by a vertical transmission, also known as “mother-to-child transmission,” from mother to an embryo, fetus or infant during pregnancy or childbirth. Examples of infection agents that may be transmitted vertically include HIV, chlamydia, rubella, Toxoplasma gondii, and herpes simplex virus. Infections may be transmitted by an iatrogenic route, referring to a transmission by medical procedures such as injection (contaminated reused needles and syringes), or transplantation of infected material, blood transfusions, or infection occurring during surgery. For example, methicillin-resistant Staphylococcus aureus (MRSA), which may cause several severe infections, may be transmitted via iatrogenic route during surgery. Infections may also be transmitted by vector-borne transmission, where a vector may be an organism transferring the infection agents from one host to another. Such vectors may be triatomine bugs, e.g. trypanosomes, parasites, animals, arthropods including e.g. mosquitos, flies, lice, flees, tick and mites or humans. Non-limiting examples of mosquito-borne infections include Dengue fever, West Nile virus related infections, Yellow fever and Chikungunya fever. Non-limiting examples of parasite-borne diseases include malaria, Human African trypanosomiasis and Lyme disease. Non-limiting examples of diseases spread by humans or mammals include HIV, Ebola hemorrhagic fever and Marburg fever.

Traditionally infectious diseases are treated with medications and/or good supportive care. Medical prevention, treatment and/or management of bacterial infections may include administration of antibiotics. Antibiotics may inhibit the colonization of bacteria or kill the bacteria. Antibiotics include e.g. penicillins, cephalosporins, macrolides, fluoroquinolones, sulfonamides, tetracyclines, and aminoglycosides. Antibiotics may be specific to a certain bacteria or act against broad spectrum of bacteria. Some types of bacteria are especially susceptible to antibiotics, whereas some bacteria are more resistant. Development of bacterial strain mutations that are resistant to antibiotics is an increasing concern. Methicillin-resistant Staphylococcus aureus (MRSA), vancomycin-resistant Enterococcus (VIDE), multi-drug-resistant Mycobacterium tuberculosis (MDR-TB) and Klebsiella pneumoniae carbapenemase-producing bacteria (KPC) are examples of bacteria that are resistant to most general antibiotics. Due to the emerging resistance, unnecessary administration and overdosing of antibiotics should be avoided. Medical prevention, treatment and/or management of viral infections may include administration of antiviral medications. Antiviral medications may be specific to a certain bacteria or act against a broad spectrum of viruses. Currently antiviral medications are available for es. HIV, influenza, hepatitis B and C. Medical prevention, treatment and/or management of viral infections may include administration of antifungal medication. Antifungal medication kills or prevents the growth of fungi. Types of antifungal medications include e.g. imidazoles, triazoles and triazoles, allylamines, and echinocandins. Development of antifungal medication capable of targeting fungal cells without affecting human cells is a challenge due to the similarities of human and fungal cell on the molecular level. Typically, medical treatment is combined with good supportive care, which includes provision of fluids, bed rest, medication to relieve pain and lower fever, supportive alternative medicine such as vitamins, antioxidants and other supplements important for wellbeing of patients.

Antibody therapies for infectious diseases have also been developed. Examples of commercial therapeutic antibodies include raxibacumab (developed by Cambridge Antibody Technology and Human Genome Sciences) which is an antibody for the prophylaxis and treatment of inhaled anthrax, SHIGAMAB™ (developed by Bellus Health Inc.) is a monoclonal antibody for treatment of Shiga toxin induced hemolytic uremic syndrome, and actoxumab and bezlotoxumab (developed by Medarex Inc. and the University of Massachusetts Medical School) are commercial human monoclonal antibodies targeting C. difficile toxin A and toxin B, respectively.

Infectious diseases and/or infection related diseases, disorders, and/or conditions that may be treated by methods, components and compositions of the present disclosure include, but are not limited to, 14-day measles, 5-day fever, acne, acquired immunodeficiency syndrome (AIDS), acrodermatitis chronica atrophicans (ACM, acute hemorrhagic conjunctivitis, acute hemorrhagic cystitis, acute rhinosinusitis, adult T-cell leukemia-lymphoma (ATLL), African sleeping sickness, alveolar hydatid, amebiasis, amebic meningoencephalitis, anaplasmosis, anthrax, arboviral, ascariasis, aseptic meningitis, Athlete's foot, Australian tick typhus, avian Influenza, babesiosis, bacillary angiomatosis, bacterial meningitis, bacterial vaginosis, balanitis, balantidiasis, Bang's disease, Barmah Forest virus, bartonellosis, bat lyssavirus, Bay sore, Baylisascaris, beaver fever, beef tapeworm, bejel, biphasic meningoencephalitis, black bane, black death, black piedra, Blackwater fever, blastomycosis, blennorrhea of the newborn, blepharitis, boils, Bornholm disease, borrelia miyamotoi disease, botulism, boutonneuse fever, Brazilian purpuric fever, break bone fever, brill, bronchiolitis, bronchitis, brucellosis, bubonic, bubonic plague, bullous impetigo, Burkholderia mallei, Burkholderia pseudomallei, burly ulcers mycoburuli ulcers, Busse-Buschke disease, California group encephalitis, campylobacteriosis, candidiasis, canefield fever, canicola fever, capillariasis, carate, carbapenem-resistant enterobacteriaceae (CRE), Carrion's disease, cat scratch fever, cave disease, central Asian hemorrhagic fever, Central European tick, cervical cancer, Chagas disease, cancroid, Chicago disease, chickenpox, Chiclero's ulcer, chikungunya fever, chlamydial, cholera, chromoblastomycosis, ciguatera, clap, clonorchiasis, Clostridium difficile, Clostridium perfringens, coccidioidomycosis fungal, coenurosis, colorado tick fever, condyloma accuminata, condyloma lata, Congo fever, Congo hemorrhagic fever virus, conjunctivitis, cowpox, crabs, Crimean disease, croup, crypto, cryptococcosis, cryptosporidiosis, cutaneous larval migrans, cyclosporiasis, cystic hydatid, cysticercosis, cystitis, Czechoslovak tick, d68 (EV-d68), dacryocytitis, dandy fever, darling's disease, deer fly fever, dengue fever types 1, 2, 3, and 4, desert rheumatism, devil's grip, diphasic milk fever, diphtheria, disseminated intravascular coagulation, dog tapeworm, donovanosis, dracontiasis, dracunculosis, duke's disease, dum dum disease, Durand-Nicholas-Favre disease, dwarf tapeworm, E. coli, eastern equine encephalitis, Ebola hemorrhagic fever, Ebola virus disease (EVD), ectothrix, ehrlichiosis, encephalitis, endemic relapsing fever, endemic syphilis, endophthalmitis, endothrix, enterobiasis, enterotoxin B poisoning (staph food poisoning), enterovirus, epidemic keratoconjunctivitis, epidemic relapsing fever, epidemic typhus, epiglottitis, epsilon toxin, erysipelis, erysipeloid, erysipelothricosis, erythema chronicum migrans, erythema infectiosum, erythema marginatum, erythema multiforme, erythema nodosum, erythema nodosum leprosum, erythrasma, espundia, eumycotic mycetoma, European blastomycosis, exanthem subitum, eyewolin, Far-Eastern tick, fascioliasis, fievre boutonneuse, fifth disease, Filatow-Dukes' disease, fish tapeworm, Fitz-Hugh-Curtis syndrome-perihepatitis, finders island spotted fever, flu, folliculitis, four corners disease, frambesia, francis disease, furunculosis, gas gangrene, gastroenteritis, genital herpes, genital warts, German measles, Gerstmann-Straussler-Scheinker (GSS), giardiasis, Gilchrist's disease, gingivitis, gingivostomatitis, glanders, glandular fever, gnathostomiasis, gonococcal, gonorrhea, granuloma inguinale, guinea worm, haemophilus influenza disease, hamburger disease, Hansen's disease, Hantaan disease, Hantaan-Korean hemorrhagic fever, hantavirus pulmonary syndrome (UPS), hard chancre, hard measles, Haverhill fever, head and body lice, heartland fever, helicobacterosis, hemolytic uremic syndrome (HUS), hepatitis A, hepatitis B, hepatitis C, hepatitis D, hepatitis E, herpangina, herpes-genital, herpes labialis, herpes-neonatal, hidradenitis, histoplasmosis, histoplasmosis, his-werner disease, hiv, hookworm s, hordeola, HTLV-associated myelopathy (HAM), human granulocytic ehrlichiosis, human monocytic ehrlichiosis, human papillomarivus (HPV), human pulmonary syndrome, human pulmonary syndrome (HPS), human T-cell lymphotropic virus (HTLV), hydatid cyst, hydrophobia, impetigo, including congenital, inclusion conjunctivitis, infantile diarrhea, infectious mononucleosis, infectious myocarditis, infectious pericarditis, influenza, isosporiasis, Israeli spotted fever, Japanese encephalitis, jock itch, jorge lobo disease, jungle yellow fever, Junin Argentinian hemorrhagic fever, kala azar, Kaposi's sarcoma, keloidal blastomycosis, keratoconjunctivitis, kuru, Kyasanur forest disease, lacrosse encephalitis, lassa hemorrhagic fever, legionellosis, legionnaires disease, legionnaire's pneumonia, Lemierre's syndrome, lemming fever, leprosy, leptospirosis, listeria, listeriosis, liver fluke, lobo's mycosis, lock jaw, lockjaw, loiasis, louping ill, Ludwig's angina, lung fluke, Lyme disease, lymphogranuloma venereum (LGV), Machupo Bolivian hemorrhagic fever, Madura foot, mal del pinto, malaria, malignant pustule, Malta fever, Marburg hemorrhagic fever, masters disease, maternal sepsis, measles, Mediterranean spotted fever, melioidosis, meningitis, meningococcal disease, Middle East Respiratory Syndrome (NIERS), methicillin-resistant Staphylococcus aureus (MIRSA), milker's nodule, molluscum contagiosum, moniliasis, monkeypox, mononucleosis, mononucleosis-like syndrome, Montezuma's revenge, morbilli, mucormycosis, multiple organ dysfunction syndrome (MODS), multiple-system atrophy (MSA), mumps, murine typhus, Murray Valley encephalitis (MVE), mycoburuli ulcers, mycotic vulvovaginitis, myositis, Nanukayami fever, necrotizing fasciitis, necrotizing fasciitis-type 1, necrotizing fasciitis-type 2, negishi, new world spotted fever, nocardiosis, nongonococcal urethritis, non-polio enterovirus, norovirus, North American blastomycosis, North Asian tick typhus, Norwalk virus, Norwegian itch, Ohara disease, Omsk hemorrhagic fever, onchoceriasis, onychomycosis, opisthorchiasis, opthalmia neonatorium, oral hairy leukoplakia, orf, oriental sore, oriental spotted fever, ornithosis, Oroya fever, otitis externa, otitis media, pannus, paracoccidioidomycosis, paragonimiasis, parainfectious, paralytic shellfish poisoning, paronychia, parotitis, parrot fever, pediculosis, peliosis hepatica, pelvic inflammatory disease, pertussis, phaeohyphomycosis, pharyngoconjunctival fever, piedra, pigbel, pink eye conjunctivitis, pinta, pinworm, pitted keratolysis, pityriasis versicolor, plague, pleurodynia, pneumococcal disease, pneumocystis pneumonia, pneumocystosis, pneumonia, polio, poliomyelitis, polycystic hydatid, Pontiac fever, pork tapeworm, Posada-Wernicke disease, postangina septicemia, Powassan, progressive multifocal leukencephalopathy (PML), progressive rubella panencephalitis, prostatitis, pseudomembranous colitis, psittacosis, puerperal fever, pustular rash diseases, pyelonephritis, pylephlebitis, q-fever, quinsy, quintana fever, rabbit fever, rabies, racoon roundworm, rat bite fever, rat tapeworm, Reiter syndrome, relapsing fever, respiratory syncytial virus (RSV), rheumatic fever, rhodotorulosis, ricin poisoning, rickettsialpox, rickettsiosis, Rift valley fever, ringworm, Ritter's disease, river blindness, rocky mountain spotted fever, rose handler's disease, rose rash of infants, roseola, Ross river fever, rotavirus, roundworm s, rubella, rubeola, Russian spring, salmonellosis gastroenteritis, San Joaquin valley fever, Sao Paulo encephalitis, Sao Paulo fever, scabies infestation, scalded skin syndrome, scalded skin syndrome, scarlatina, scarlet fever, schistosomiasis, scombroid, scrub typhus, sennetsu fever, sepsis, septic shock, severe acute respiratory syndrome, severe acute respiratory syndrome (SARS), shiga. toxigenic Escherichia coli, shigella, shigellosis gastroenteritis, shinbone fever, shingles, shipping fever, siberian tick typhus, sinusitis, sixth disease, slapped cheek disease, sleeping sickness, small pox, smallpox, snail fever, soft chancre, southern tick associated rash illness, sparganosis, Spelunker's disease, sporadic typhus, sporotrichosis, spotted fever, spring, St. Louis encephalitis, staphylococcal food poisoning, staphylococcal, strep. throat, streptococcal disease, streptococcal toxic-shock syndrome, strongyloiciasis, stye, subacute sclerosing panencephalitis (SS APE), sudden acute respiratory syndrome, sudden rash, swimmer's ear, swimmer's itch, swimming pool conjunctivitis, sylvatic yellow fever, syphilis, systemic inflammatory response syndrome (SIRS), tabes dorsalis, taeniasis, taiga encephalitis, tanner's disease, tapeworm s, temporal lobe encephalitis, tertiary syphilis, tetani, tetanus, threadworm s, thrush, tick, tick typhus, tinea barbae, tinea capitis, tinea corporis, tinea cruris, tinea manuum, tinea nigra, Tinea pedis, tinea unguium, tinea versicolor, torulopsosis, torulosis, toxic shock syndrome, toxoplasmosis, transmissible spongioform, traveler's diarrhea, trench fever 5, trichinellosis, trichomoniasis, trichomycosis axillaris, trichuriasis, tropical spastic paraparesis (TSP), trypanosomiasis, tuberculosis (TB), tularemia, typhoid fever, typhus fever, ulcus molle, undulant fever, urban yellow fever, urethritis, vaginitis, vaginosis, valley fever, vancomycin intermediate (VISA), vancomycin resistant (VRSA), varbuncle, varicella, variola, varrion's disease, venezuelan equine encephalitis, Verruga peruana, vibrio, Vibrio cholerae, vibriosis, vincent's disease or trench mouth, viral conjunctivitis, viral meningitis, viral meningoencephalitis, viral rash, visceral larval migrans, vomito negro, vulvovaginitis, warts, Waterhouse, Weil's disease, West Nile fever, Western equine encephalitis, Whipple's disease, whipworm, white piedra, whitlow, Whitmore's disease, whooping cough, winter diarrhea, wolhynia fever, wool sorters' disease, yaws, yellow fever, yersinosis, zahorsky's disease, zika virus disease, zoster, zygomycosis, acute bacterial rhinosinusitis, lobomycosis, and/or any other infectious diseases, disorders or conditions.

### John Cunningham Virus (JCV)

John Cunningham Virus is a common human polyomavirus. The transmission route of JCV is unknown. The virus is suspected to be spread by contaminated water and may be Obtained through tonsils or by the gastrointestinal tract. 70-90% of humans are estimated to be infected by the virus, and for normal healthy individuals the infection is asymptomatic. However, for patients with weakened immune system, KW may lead to Progressive multifocal leukoencephalopathy (PML). PML is a condition characterized by multifocal progressive damage or inflammation of the white matter of the brain. The symptoms include clumsiness, progressive weakness and changes in visual, speech and personality. PLM has a mortality rate of 30-50% and patients who survive the disease are left with severe neurological disabilities. PML occurs in patients with a severe immunodeficiency, most commonly in patients with HIV/AIDS. As many as 5% of HIV/AIDS patients are affected by PML. Individuals with other autoimmune conditions such as multiple sclerosis, rheumatoid arthritis, and systemic lupus erythematosus are also at risk, as well as individuals going through immunosuppressive therapy for cancer, e.g. lymphoma or Hodgkin's disease, or organ transplant. PML associated with immunosuppressive therapy is an increasing concern. For example, commercial antibody natalizumab (TYSABRI®, developed by Biogen Idec) for treatment of multiple sclerosis increases susceptibility to PML. Other drugs associated with increased risk of PML include Rituximab (JURAAN®, developed by DEC Pharmaceuticals), Efalizumab (RAPTIVA® developed by Genentech and XOMA) and Mycophenolate mofetil (CELLCEPT®, developed by Genentech).

JCV is a nonenveloped, T=7 icosahedral virus with a closed circular, double-stranded DNA genome. The major capsid component is the viral protein VP1 is made of 72 pentamers formed by VP1 monomers linked through the C terminal end. VP1 starts the infection by binding to the receptor target cells. After initial infection, typically occurring in childhood or adolescence, the virus stays quiescent in the kidneys and the lymphoid organs. In healthy individuals, the virus may replicate in kidney without causing any symptoms. However, in patients with weakened immune system, JCV may cross the blood-brain barrier into the central nervous system causing PIL.

As of today, there is no known cure for PML. Current therapies focus on reversing the immune deficiency to slow down or stop the progress of the disease. There remains a need for therapies neutralizing JVC for prevention, management and treatment of JCV infection and PML Goldmann et al. demonstrated that neutralizing activity with JCV VP1 protein in sera of a rabbit (see Goldmann C. et al., 1999, J Virol. 73(5): 4465-4469). Therapies based on neutralizing JCV antibodies could be applied for treatment, management and/or prevention of PML. Recently, immunological approaches have been under investigation and neutralizing antibodies binding to JC virus, especially targeting the VP1 protein, have been developed e.g. as described in US Patent Publication US2015/0191530, US2015/0056188 and US2015/0050271, the contents of each of which are incorporated herein by reference in their entirety. Such antibodies may cause reduction of JCV replication, proliferation or infectivity. Antibodies may bind to a conformational epitope of JCV VP1 protein or to the sialic acid binding pocket of VP 1 protein of JCV.

In some embodiments, methods of the present disclosure may be used to prevent, manage and/or treat JCV infection and/or PML.

Viral particles and methods of using the viral particles described in the present disclosure may be used to prevent, manage and/or treat JCV. As a non-limiting example, the viral particles of the present disclosure comprise a nucleic acid sequence encoding at least one of the sequences described in Table 37.

### Influenza Virus

Influenza viruses cause a common respiratory infection called influenza (flu). Influenza viruses are categorized into three main groups, virus A, B and C. Influenza viruses are negative-sense, single-stranded, segmented RNA viruses. Influenza A contains two proteins on the surface of the viral envelope: hemagglutinin (H), which is a protein responsible for red blood cell agglutination and neuraminidase (N), which is an enzyme cleaving the glycosidic bonds of neuraminic acid. Influenza A mutates at a faster rate than types B and C. Several serotypes of H and subtypes of N have been identified. Influenza Type B, similarly to Type A, contains H and N protein. Type C influenza virus is a single stranded RNA virus with glycoprotein called hemagglutinin-esterase fusion. Influenza strains vary according to geographical presentation.

Influenza in general is a highly contagious disease and may be transmitted by the respiratory route. Influenza symptoms include e.g. high fever, runny nose, headache, sore throat, muscle pain, cough and occasionally nausea and vomiting. Influenza may lead to other complications such as pneumonia or sinus infections. Influenza may be dangerous to young children, the elderly, pregnant women and individuals with chronic medical conditions or weakened immune system. According to Centers for Disease Control and Prevention (CDC), the estimated annual number of flu-associated deaths in the United States ranges between 3000 and 49,000, depending on the severity of the seasonal variations.

Influenza may be treated with good supportive care and antiviral medication. Antiviral medications include neuraminidase inhibitors, e.g. oseltamivir and zanamivir and M2 protein inhibitors. However, some strains of influenza appear to be resistant to these antiviral medications. Seasonal vaccinations to influenza are very efficient in prevention of the disease and are recommended annually.

There remains a need for prevention and treatment therapies for influenza, especially for those providing long lasting and broad neutralization. Therapeutic antibodies against influenza viruses have been developed. In general, antibody responses to different subtypes and serotypes of influenza A, B and C are unique. Some therapeutic antibodies are specific to an antibody type, whereas some have a broad coverage. Navivumab (developed by Celltrion, Inc.) taught in US Patent application US20140234336, firivumab (developed by Celltrion, Inc.) taught in US Patent application US20130004505 and diridavumab (developed by Jansen Biotech, Crucell and Johnson&Johnson) taught in International Patent application WO/2008/028946 are examples of therapeutically antibodies against influenza A hemagglutinin HA.

Viral particles and methods of using the viral particles described in the present disclosure may be used to prevent, manage and/or treat influenza. As a non-limiting example, the viral particles of the present disclosure comprise a nucleic acid sequence encoding at least one of the sequences described in Table 32.

### Hepatitis

Hepatitis is an inflammation of the liver. Hepatitis may be caused by an infection of hepatitis viruses A, B, C, D or E. In some cases, hepatitis may be asymptotic, A typical symptom of hepatitis is jaundice, characterized by yellowing of the skin, mucous membrane and conjunctiva. Other symptoms include loss of appetite, diarrhea, nausea and fever. Hepatitis may lead to a liver failure. Acute form of hepatitis is healed within six months of infection. The inflammation may also progress to a chronic hepatitis, which may lead to liver complications such as fibrosis, cirrhosis or hepatocellular carcinoma. There is no specific treatment for hepatitis. Typically, acute hepatitis is treated with good supportive care, including good nutritional balance, fluid and rest. Chronic hepatitis may be treated with antiviral drugs. Hepatitis may be prevented by vaccinations.

Hepatitis A (HAV) virus belongs to the family of Picornaviridae. HAV is encapsidated in an icosahedral structure formed by 60 copies of three viral structural proteins (VP1, VP2 and VP3), (see e.g. Kim et al. 2004, Virology.; 318(4598-607, and references therein). HAY is spread by the fecal-oral-route. Typical transmission is through contaminated food or drink or in contact with an infected individual. Improperly cooked shellfish is a common source of HAV. Hepatitis A is more abundant in developing countries with poor sanitary conditions. According to the World Health Organization (WHO), an estimated 1.4 million people are infected by HAV every year.

Vaccines for prevention of HAV infection exists and are recommended to be administered to children under 1 year of age by CDC, As of today, there is no specific treatment for HAY infection. The treatment includes supportive therapy and may last for weeks or even months. There remains a need for treatment therapies for HAV, Antibodies for prevention and/or treatment of HAY have been developed. For example, US Patent US763476, International Publication WO2011114353 and Kim et al in Virology. 2004 Jan. 20; 318(2):598-607, the contents of each of which are incorporated herein by reference in their entirety, teach neutralizing antibodies targeting HAY antigens.

Hepatitis B (HBV) belongs to the family of Orthohepadnaviridae. HBV comprises a 3.2 kb-partially double-stranded circular DNA genome. HBV virus may be transmitted via the sexual transmission route, vertical transmission at birth, iatrogenic route (e.g. blood transfusions, contaminated reused needles and syringes), as well as via exposure to certain body fluids of an infected individual. According to the WHO, an estimated 240 million people are chronically infected with hepatitis B annually, and more than 780 000 people die to associated complications.

HBV may be prevented by vaccination. The WHO recommends vaccination for all infants, as well as for adults living in increased risk of the infection. HBV infection may be treated with antiviral medications, e.g. tenofovir and entecavir. The medication does not cure the disease but suppresses the replication of the virus. Individuals with chronic hepatitis B infection are administered antiviral medications for life. There remains a need for therapies providing long lasting management and/or cure for HBV infection. Antibodies for prevention and/or treatment of HBV infection are described e.g. in US Patent publication US20120308580 and International publication WO2013165972, the contents of each of which are herein incorporated by their reference in their entirety.

Hepatitis C (HCV) belongs to the family of Flaviviridae HCV is a positive-sense single-stranded RNA virus with an open reading frame with 9600 nucleotide bases. HCV is most commonly transmitted by the sexual transmission route or iatrogenic route. Hepatitis C may be transmitted also via the vertical route, though uncommon. According to WHO, 130-150 million people have a chronic HCV infection and approximately half a million people die from complications associated with HCV annually.

As of today, there is no vaccine for I-ICY infection. Traditional treatment of hepatitis C is based on antiviral medication therapy with e.g. ribavirin and interferon. More recently, direct antiviral agents (DAA) have been developed to treat hepatitis C infections. However, there remains a need for efficient prevention and treatment therapies for HCV infection.

Hepatitis D (HDV) is a small spherical enveloped RNA virus belonging to the genus of deltaviruses. HDV infection may only replicate in the presence of a HBV virus and therefore HDV infection has a dependency on HBV. HMI virus may be transmitted as coinfection with HBV or be superimposed on chronic HBV or HBV carrier state. HDV may be transmitted similarly to HBV, e.g. via the sexual transmission route, vertical transmission at birth, iatrogenic route, as well as via exposure to certain body fluids of an infected individual. Treatment and vaccination against HBV may be applied against HDV, and there remains a need for therapies to cure both infections.

Hepatitis E (HEY) is a linear, monoparte, single-stranded RNA virus belonging to the family of Hepeviridae. HEY may be transmitted via the fecal-oral route due to contaminated food or beverage, the iatrogenic route (e.g. blood transfusions, contaminated reused needles and syringes) or the vertical transmission route during pregnancy. Contaminated drinking water is the most common source of infection. Improperly cooked shellfish are a common source of HEY. The disease is present worldwide but is more abundant in East and South Asia, and especially in environments with poor sanitation and hygiene. According to WHO, an estimated 20 million HEV infections occur annually leading to 56 600 death associated with HEV complications.

There is no specific treatment for BEV. The disease is typically cured with good supportive care. As of today, vaccinations against HEV are not globally available, though development in the field has been done. There remains a need for prevention and treatment therapies for HEV infection. Antibodies for prevention and treatment of REV have been developed. For example, neutralizing antibodies targeting HRV have been taught in U.S. Pat. No. 7,148,323, Tang et al. 2011, Proc. Nod. Acad. Sri. U.S.A. 108 (25), 10266-10271 and Gu et al. 2015, Cell Res. 25 (5), 604-620, the contents of each of which are incorporated herein by reference in their entirety.

In some embodiments, methods of the present disclosure may be used to prevent, manage and/or treat infections and complications caused by HAV, HBV, HCV, HDV and/or HEV.

Viral particles and methods of using the viral particles described in the present disclosure may be used to prevent, manage and/or treat HAV. As a non-limiting example, the viral particles of the present disclosure comprise a nucleic acid sequence encoding at least one of the sequences described in Table 17.

Viral particles and methods of using the viral particles described in the present disclosure may be used to prevent, manage and/or treat HBV. As a non-limiting example, the viral particles of the present disclosure comprise a nucleic acid sequence encoding at least one of the sequences described in Table 34.

Viral particles and methods of using the viral particles described in the present disclosure may be used to prevent, manage and/or treat HDV. As a non-limiting example, the viral particles of the present disclosure comprise a nucleic acid sequence encoding at least one of the sequences described in Table 34.

Viral particles and methods of using the viral particles described in the present disclosure may be used to prevent, manage and/or treat HEV. As a non-limiting example, the viral particles of the present disclosure comprise a nucleic acid sequence encoding at least one of the sequences described in Table 17.

### Respiratory Syncytial Virus (RST)

Respiratory syncytial virus (RSV) is a single-stranded RNA virus belonging to the family of Paramyxoviridae. The RSV RNA is contained in a nucleocapsid made of 11 proteins and covered with a lipid envelope (see, e.g. Piedimonte, 2015, Cleve Clin J Med.; 82(11 Suppl 1):S13-8, and references therein). RSV attaches to the epithelial cells of the host airway cells with the surface glycoproteins G and F and merges the viral envelope to the membranes of adjacent cells. G and F glycoproteins are the principal antigens exposed to the host immune system.

Respiratory syncytial virus (RSV) causes infections of the respiratory tract including the lungs and breathing passages. RSV is transmitted through the respiratory transmission route, in direct contact with nasal or oral secretions of infected individuals, or indirectly e.g. by touching a contaminated surface. The symptoms include a runny nose, decrease of appetite, coughing, sneezing, fever and wheezing. The infection may progress into a pneumonia or bronchiolitis. Additionally, RSV infection may have a role in triggering asthma attacks and in the inception of asthma for individuals with a family history of asthma. In healthy adults, RSV infection is typically mild and does not require hospitalization. However, the infection may be dangerous for young children and infants, and for individuals with a weakened immune system. According to the CDC, almost all children under 3 years of age will acquire an RSV infection and up to 2% of cases require hospitalization. RSV infection the most common cause for bronchiolitis and pneumonia in children younger than 1-year-old.

As of today, there is no specific medical treatment for RSV infection on the market and typically the infection is treated with good supportive care. There remains a need for prevention and treatment therapies for RSV infections and associated complications. Antibodies for treatment and prevention of RSV infection have been developed. For example, palivizumab (developed by MedImmune) taught in U.S. Pat. No. 8,153,133, the contents of which are incorporated herein by reference in their entirety, is a nearly human monoclonal antibody targeting the RSV F glycoprotein. Palivizumab is used for passive immunity for infants at risk for severe infection, including children with hemodynamically significant congenital heart defects, profound immunodeficiency and pulmonary or neuromuscular pathologies impairing airway clearance.

In some embodiments, methods of the present disclosure may be used to prevent, manage and/or treat infections and complications caused by RSV.

Viral particles and methods of using the viral particles described in the present disclosure may be used to prevent, manage and/or treat RSV. As a non-limiting example, the viral particles of the present disclosure comprise a nucleic acid sequence encoding at least one of the sequences described in Table 33.

### Herpes Simplex Virus 1 and 2

Herpes simplex viruses 1 and 2 (HSV1 and HSV2), also known as human herpesvirus 1 and 2 (HHV-1 and HHV-2), belong to the family of Herpesviridae Herpesviruses in general, consist of an icosahedral capsid surrounded by a membrane envelope. The capsid contains the viral double stranded DNA. The capsid is surrounded by an amorphous tegument of 30 viral proteins. The virion is enveloped by lipids with multiple viral glycoproteins and cellular proteins (see, e.g. McAllister and Schleiss, 2014, Expert Rev Vaccines.; 13(11): 1349-1360, and references therein).

HSV1 and HSV2 cause an infection known as herpes, which is characterized by blisters in the skin, or mucous membranes of the mouth, lips, also known as “cold sores”, or genitals. Typically, the symptoms are mild or asymptomatic. However, HSV1 and HSV2 are neurotropic and neuroinvasive viruses persisting in the body by becoming latent, and sustain in the cell bodies of neurons. The infection is lifelong with outbreaks, or sporadic episodes of viral reactivation, when the virus in the nerve cells become active causing new blistering. The infection may be dangerous to individuals with weakened immune system. Neonatal herpes of infants may be fatal. Occasionally HSV1 infections may lead to encephalitis or keratitis. HSV1 and HSV2 are transmitted by contact with an infected area during reactivations of the virus. HSV1 is mainly transmitted by oral-to-oral contact, skin contact or the sexual transmission route. HSV1 may also be transmitted vertically during birth. HSV2 is transmitted via the sexual transmission route and is one of the most common sexually transmitted infections. According to the WHO, an estimated 67% of world's population aged under 50 years has an HSV-1 infection. An estimated 11% of world's population aged 15-49 years has an HSV2 infection.

As of today, there is no vaccination for prevention of HSV1 and HSV2 infections on the market. HSV1 and HSV2 infections may be treated with antiviral medications, such as acyclovir, famciclovir and valacyclovir. Antiviral medications do not cure the infection, but reduce the severity and frequency of symptoms. There remains a therapy for prevention and cure for these infections. Antibodies for prevention, treatment and management of HSV1 and HSV2, targeting the viral glycoproteins, have been developed, as described e.g. in U.S. Pat. Nos. 8,431,118, 5,646,041, Haynes US Patent Publication US2014/0302062, the contents of each of which are incorporated herein by reference in their entirety.

In some embodiments, methods of the present disclosure may be used to prevent, manage and/or treat infections and complications caused by HSV1 and HSV2.

Viral particles and methods of using the viral particles described in the present disclosure may be used to prevent, manage and/or treat HSV. As a non-limiting example, the viral particles of the present disclosure comprise a nucleic acid sequence encoding at least one of the sequences described in Table 35.

### Human Cytomegalovirus

Human Cytomegalovirus (HCMV) also known as human herpesvirus 5 (HHV-5) belongs to the family of Herpesviridae, a sub-family of Betaherpesvirinae. HCMV is a double-stranded DNA enveloped virus composed of a nucleocapsid surrounded by structured tegument layer and bounded by a trilaminate membrane envelope.

In most occasions, an initial HCMV infection is asymptomatic, or associated with mild symptoms e.g. sore throat, fatigue, flu-like symptoms, and fever. After initial infections, HCMV virus resides in mononuclear cells without detectable symptoms. HCMV infection may be dangerous to individuals with weakened immune system. HCMV may be transmitted by contact with certain body fluids of an infected individuals (e.g. saliva, urine, semen). HCMV may be transmitted vertically, especially if acquired during pregnancy, leading to a congenital HCMV infection. According to CDC, about 1 in 150 children are born with congenital CMV infection. In about 20% of cases, congenital HCMV infection may lead to premature birth, birth defects or developmental disabilities, e.g. liver, lung, spleen problems, small head size, small body size or seizures.

As of today, there is no specific treatment or prevention therapy for HCMV infection. In severe cases of congenital HCMV infection, infants may be treated with an antiviral drug, ganciclovir, to prevent hearing loss and developmental outcomes. However, the drug has serious side effects. There remains a need for prevention therapy and improved therapies for treatment and cure of HCMV infection. Antibodies neutralizing HCMV have been developed. Such antibodies are taught e.g. in International Patent Publication WO2010007463, U.S. Pat. No. 59,149,524, US8492529 and 58202518, the contents of each of which are incorporated herein by reference in their entirety.

In some embodiments, methods of the present disclosure may be used to prevent, manage and/or treat infections and complications caused by HCMV.

Viral particles and methods of using the viral particles described in the present disclosure may be used to prevent, manage and/or treat HCMV. As a non-limiting example, the viral particles of the present disclosure comprise a nucleic acid sequence encoding at least one of the sequences described in Table 35.

### Epstein-Farr Virus

Epstein-Barr virus (EBV), also known as human herpesvirus 4 (HHV-4) belongs to the family of Herpesviridae, EBV is a double-stranded DNA virus composed of a protein nucleocapsid surrounded by a tegument layer and bounded by an envelope containing lipids and surface projection of glycoproteins. EBV may enter B cells and epithelial cells.

EBV infection causes glandular fever known as infectious mononucleosis, also known as the kissing disease. Typical symptoms include e.g. sore throat, fever swollen lymph nodes in the neck, enlarged spleen, swollen liver, rash and fatigue, Additionally, EBV infection is associated with certain cancers, e.g. central nervous system lymphomas, Hodgkin's lymphoma, Burkitt's lymphoma, Guillain-Barre syndrome, multiple sclerosis, and higher susceptibility to certain autoimmune diseases. The virus is transmitted via contact with certain bodily fluids of an infected individual, especially through saliva. The infection affects majority of population. According to CDC, 90% of adult population have antibodies demonstrating current or past EBV infection.

As of today, there is no specific therapy for prevention or treatment of EBV infection on the market. Typically, EBV infection is treated with good supportive care. Antibodies for prevention, management and treatment of EBV infection and associated diseases have been developed, e.g. by Wang and Fogg in US Patent publication US20150064174 and Fang et al. in Intervirology 50 (4), 254-263 (2007), the contents of each of which are incorporated herein by reference in their entirety.

In some embodiments, methods of the present disclosure may be used to prevent, manage and/or treat infections and complications caused by EBV.

Viral particles and methods of using the viral particles described in the present disclosure may be used to prevent, manage and/or treat EBV. As a non-limiting example, the viral particles of the present disclosure comprise a nucleic acid sequence encoding at least one of the sequences described in Table 42.

### Varicella Zoster Virus

Varicella. zoster virus (VZV), also known as human herpes virus 3 (HHV-3) and chickenpox virus, belongs to the family of Herpesviridae. VZV is a linear duplex DNA molecule containing two segments (L and 5) joined covalently. At least five clades of the virus have been identified.

VZV causes varicella, also known as chickenpox, which is an infection characterized by blister-like rash, itching, fatigue and fever. Chickenpox may be dangerous for babies, adults and individuals with weakened immune system. After primary phase of the infection, VZV resides in the nerves, including cranial nerve ganglia, dorsal root ganglia and autonomic ganglia, and may eventually lead to shingles, which is a viral disease characterized with a painful skin rash, blistering and occasionally nerve pain, Additionally, VZV has been associated with other complications, e.g, neurological conditions, inflammation of arteries, myelitis, Ramsay Hunt syndrome, Mollaret's meningitis. VZV is transmitted by direct contact or by the respiratory route. VZV is highly contagious. According to CDC, before WV vaccination, about 4 million people would be affected by chickenpox in the US annually, with more than 10,000 hospitalized.

VZV infection may be prevented by a vaccination, which is recommended by CDC to all children and unvaccinated adults. Chickenpox may be treated with antiviral medications, e.g. acyclovir, valacyclovir and famciclovir, or with other symptom relieving medications and therapies. However, the present antiviral medications may have undesirable side effects. There remains a need for improved therapies to treat VZV infection, and its reactivation stages. Antibodies targeting VZV have been developed, e.g. as described in U.S. Pat. No. 5,506,132, and US Patent application US20100074906, the contents of which are herein incorporated by their reference in their entirety.

In some embodiments, methods of the present disclosure may be used to prevent, manage and/or treat infections and complications caused by VZV.

Viral particles and methods of using the viral particles described in the present disclosure may be used to prevent, manage and/or treat VZV. As a non-limiting example, the viral particles of the present disclosure comprise a nucleic acid sequence encoding at least one of the sequences described in Table 42.

### Coronaviruses

Coronaviruses are a diverse group of enveloped viruses belonging to the family of Coronaviridae. Coronaviruses contain an envelope, a helical capsid, and a single-stranded, positive-sense RNA genome. Coronaviruses have a characteristic structure with viral spike-shaped glycoprotein populating the surface of the virus and causing an appearance resembling the solar corona. Coronaviruses are a common cause of mammalian and avian infections causing upper respiratory tract, gastrointestinal and central nervous system diseases.

Human coronavirus 229E, OC-43, NL63, and HKU1 are a cause a behind typical, short term ‘common cold’ and affect individuals all over the world. Typical symptoms of the infections include coughing, sneezing, fatigue and fever. Occasionally the viruses can cause lower-respiratory tract illnesses, such as pneumonia. The viruses are spread by direct contact or by the respiratory route. The infections may be dangerous to the elderly and individuals with weakened immune system. There is no specific treatment or prevention therapy for these coronavirus infections.

Severe Acute Respiratory Syndrome coronavirus (SARS-CoV) causes a viral respiratory illness. Typical symptoms of the infection include a high fever, headache, body aches, dry coughing and eventually pneumonia. SARS-CoV was identified in 2003 in an outbreak starting from Asia. SARS-CoV is transmitted by direct contact with an infected individual or by the respiratory route. According to the WHO, during the 2003 outbreak of SARS-CoV, 8098 people worldwide were infected with symptoms and out of them, 774 died. As of today, there is no specific treatment or prevention therapy for SARS on the market. Antiviral medication and steroids may be prescribed to certain patients. Antibodies targeting SARS-CoV have been developed, e.g. as described in U.S. Pat. No. 7,728,110 and US Patent publication US20110159001, the contents of each of which are herein incorporated by their reference in their entirety.

Middle East Respiratory syndrome coronavirus (MFRS-CoV) causes an acute severe respiratory infection affecting the lungs and breathing tubes. MERS-CoV was identified in 2012. Typical symptoms include fever, cough and shortness of breath, eventually pneumonia and additionally gastrointestinal symptoms. MERS-CoV is highly dangerous to humans. According to the WHO, 36% of the infections are fatal. MERS-CoV is a zoonotic virus transmitted to humans from animals, e.g. bats and camels, or from human to human. Camels are suggested to be a reservoir for MERS-CoV. Majority of MERS-CoV infection have occurred in the Arabian Peninsula, and especially in Saudi Arabia. As of today, no specific treatment of prevention therapy for MERS-CoV infection is available on the market. Antibodies targeting MERS-CoV have been developed, e.g. as described in International publication WO2015057942, the contents of which are herein incorporated by their reference in their entirety.

In some embodiments, methods of the present disclosure may be used to prevent, manage and/or treat infections and complications caused by SARS-CoV, MERS-CoV and/or other coronaviruses.

Viral particles and methods of using the viral particles described in the present disclosure may be used to prevent, manage and/or treat coronaviruses. As a non-limiting example, the viral particles of the present disclosure comprise a nucleic acid sequence encoding at least one of the sequences described in Table 36.

### Poxviruses

Poxviruses affecting humans include orthopoxvirus, parapoxvirus, yatapoxvirus and mollusipoxvirus. Poxviruses are typically brick-shaped, enveloped, single, liner or double-stranded viruses with DNA genome. Typically, poxvirus infections cause lesions, skin nodules, or disseminated rash. Poxviruses may be transmitted by direct contact with contaminated humans, animals or materials. Diseases caused by poxviruses include e.g. smallpox, monkeypox, molluscum conagiosum, vaccinia virus and orf virus infection.

Smallpox virus infection is highly fatal, and though it does not occur in nature anymore, smallpox virus is considered to be a potential chemical or biological warfare agent. The threat of terrorism has created a need for efficient and improved methods for treatment and/or prevention of smallpox infection. The traditional vaccination for smallpox, also applicable against monkeypox, has a rare but severe side effect due to vaccinia virus, which is the active constituent of the vaccine that eradicated smallpox. Vaccinia Immune Globulin (VIG) is the only licensed therapeutic treatment for smallpox, but is highly variable and available in limited quantities. Antibodies against smallpox have been developed, as described e.g. in U.S. Pat. No. 8,623,370 and US Patent publication US20140186370, the contents of each of which are herein incorporated by their reference in their entirety.

In some embodiments, methods of the present disclosure may be used to prevent, manage and/or treat infections and complications caused by smallpox virus and/or other poxviruses.

Viral particles and methods of using the viral particles described in the present disclosure may be used to prevent, manage and/or treat poxvirus. As a non-limiting example, the viral particles of the present disclosure comprise a nucleic acid sequence encoding at least one of the sequences described in Table 38.

### Enterovirus 71

Enterovirus 71 (EV71) belongs to the family of Picornaviridae. Enterovirus 71 is a single-stranded RNA positive sense virus. The virus has approximately 7411 nucleotides. The RNA genome is enclosed in an icosahedral capsid of structural proteins VP1-VP4. (see, e.g. Tan et al., 2014, J Biomed Sci; 21(1): 140, and references therein).

EV71 infections typically cause hand, foot and mouth (HFMD), which is characterized by fever, mouth ulcers, and vesicles on the palms of the hands and feet. Additionally, EV71 causes severe neurological manifestations, including poliomyelitis-like acute flaccid paralysis, brainstem encephalitis in infants and children. These neurological manifestations may be fatal, or cause permanent neurological consequences, such as delayed neurodevelopment or reduced cognitive function in children. EV71 is transmitted through direct contact with certain bodily fluids, such as saliva, or the respiratory route, or the fecal-to-mouth route. Outbreaks of EV71 have been reported by WHO in the US, Europe, and more frequently in Asia-Pacific region in the past 30 year.

As of today, no specific treatment or prevention therapy for EV71 is on the market. Antiviral drugs, e.g. pleconaris and other capsid-function inhibitors (see, e.g. Tan et al. a Biomed Sci. 2014; 21(1): 140), may be prescribed against EV71 infections, though their effectiveness is not swell established. There remains a need for prevention and treatment therapies for EV71 infection. Antibodies neutralizing EV71 have been developed. Non-limiting examples include the anti-EV71 antibody MAB979 (developed by Merck Millipore) and those taught by Carderosa et al. in International Patent Publication WO2015092668, the contents of which are incorporated herein by reference in their entirety.

In some embodiments, methods of the present disclosure may be used to prevent, manage and/or treat infections and complications caused by EV71.

Viral particles and methods of using the viral particles described in the present disclosure may be used to prevent, manage and/or treat EV71. As a non-limiting example, the viral particles of the present disclosure comprise a nucleic acid sequence encoding at least one of the sequences described in Table 39.

### Rubella Virus

Rubella virus belongs to the family of Togaviridae. Rubella virus is a positive sense, single-stranded RNA virus with spike-like, hemagglutinin containing surface projections. The virus core is enveloped by glycosylated E1 and E2 proteins.

Rubella, also known as German measles or three-day measles, is a viral infection typically characterized by a rash, low fever, nausea, swollen lymph glands behind the ears and the neck, and mild conjunctivitis. At later stage, the infection may develop arthritis and pain in the joints. Typical symptoms of rubella infection are mild and affect children and young adults. Rubella virus is transmitted by the respiratory route and the virus replicates in the nasopharyngeal mucosa and local lymph nodes. However, when an infection is acquired during pregnancy, the virus is transmitted through vertical route with 90% chance and may cause fetal death or congenital defects known as congenital rubella syndrome (CRS), Infants with CRS may have hearing impairments, eye and heart defects, diabetes mellitus, thyroid dysfunction and/or autism. According to the WHO, about 10,000 infants with CRS are born every year, majority occurring in countries with low vaccine coverage.

As of today, there is no specific treatment for rubella. Rubella may be prevented with vaccination, and rubella has been part of the vaccination program for the past 40 years. However, the infection still persists and an increasing concern related to the life-time of vaccine efficiency exists. There remains a need for long lasting prevention therapy, as well as treatment for rubella virus infection. Antibodies against rubella have been described e.g. in US Patent US20100143376, the contents of each of which are herein incorporated by reference in their entirety.

In some embodiments, methods of the present disclosure may be used to prevent, manage and/or treat infections and complications caused by rubella.

Viral particles and methods of using the viral particles described in the present disclosure may be used to prevent, manage and/or treat Rubella. As a non-limiting example, the viral particles of the present disclosure comprise a nucleic acid sequence encoding at least one of the sequences described in Table 40.

### Human Papilloma Virus

Human papilloma virus (HMO is a non-enveloped double-stranded DNA virus belonging to the family of Papillomaviridae. Over 170 types of HPV have been identified.

HPV infections may be asymptomatic, or cause infection related to warts (e.g. plantar, flat or anogenital warts), oral infections such as papillomas or multifocal epithelial hyperplasia. The infection may be undetected, and clears from the body to low levels within two years. Infections caused by human papillomavirus (HPV) have been associated with certain cancers of stratified epithelial tissues, e.g. cervical, anal, vaginal, vulvar and penile cancers, lung and throat cancers. Especially HPV16 and HPV18 are known to be carcinogenic. According to the WHO, persistent genital HPV infection may cause cervical cancer which is the second most common cancer in women worldwide. In developing countries, cervical cancer counts for 13% of all female cancers, and survivor rate worldwide is approximately 50%. HPV is very common. CDC estimates that every one in four individuals in the US has an HPV infection. Most commonly HPV is transmitted by the sexual route, but also the vertical transmission route, or by direct contact to infected blood, or objects may occur.

Cancers caused by HPV may be prevented by vaccines developed against certain HPV types. The vaccines are available worldwide and are recommended by CDC for all preteen aged children. As of today, there are no specific treatment for HPV infection. There remains a need for prevention and treatment therapy affecting a broad range of HPV infections. Antibodies for HPV have been developed, e.g. as described in International publication WO2015096269, the contents of each of which are herein incorporated by reference in their entirety.

In some embodiments, methods of the present disclosure may be used to prevent, manage and/or treat infections and complications caused by HPV.

Viral particles and methods of using the viral particles described in the present disclosure may be used to prevent, manage and/or treat HPV. As a non-limiting example, the viral particles of the present disclosure comprise a nucleic acid sequence encoding at least one of the sequences described in Table 41.

### Pseudomonas Aeruginosa

Pseudomonas Aeruginosa (P. Aeruginosa) is a common Gram-negative, aerobic, rod-shaped bacterium belonging to the family of Pseudomonodaceae. P. aeruginosa is found in soil, water, skin, flora, and in most manmade environments around the world. P. aeruginosa is considered as an opportunistic pathogen taking advantage of a weakened immune system.

P. aeruginosa may cause a variety of mild infections, such as, urinary tract infections, respiratory system infections, dermatitis, soft tissue infections, bacteremia, bone and joint infections, gastrointestinal infections, blood infections, ear infections, skin rash, eye infections and a variety of systemic infections. P. aeruginosa is transmitted through water, contaminated hands, materials or objects. In general, P. aeruginosa infections in healthy individuals are very mild or asymptomatic. However, the infections expose a significant risk for individuals with weakened immunity, such as patients with other underlying illnesses or complications, and especially when in a hospital environment. For example, patients with cystic fibrosis have a susceptibility towards loss of lung function due to respiratory tract infection with the bacterium. Patients attached to breathing machines, patients with catheters, or with surgery wounds or burn wounds are potentially at risk for serious and life-threatening infections. P. aeruginosa infection may lead to a fatal sepsis. According to CDC, approximately 51, 000 healthcare associated infection occur in the US every year, leading to approximately 400 deaths.

As of today, there are no prevention therapies for P. aeruginosa infection on the market. Some strains of P. aeruginosa may be treated with antibiotics, e.g. gentamicin, tobramycin, colistin, and amikacin. However, an increasing number of strains of P. aeruginosa, especially those affecting hospitalized patients, are resistant to antibiotics and no specific treatment therapy exists. There remains a need for improved treatment and prevention therapies against P. aeruginosa infections. Antibodies against P. aeruginosa have been developed, such as, panobacumab (developed by Kenta Biotech Inc), which is an antibacterial antibody against P. Aeruginosa.

In some embodiments, methods of the present disclosure may be used to prevent, manage and/or treat infections and complications caused by P. aeruginosa.

Viral particles and methods of using the viral particles described in the present disclosure may be used to prevent, manage and/or treat P. aeruginosa. As a non--limiting example, the viral particles of the present disclosure comprise a nucleic acid sequence encoding at least one of the sequences described in Table 43.

### Streptococcus Bacteria

Streptococcus is a genus of gram-positive bacteria belonging to the family of Streptococcaceae. Species of Streptococcus are divided into alpha- and beta-hemolytic species. Alpha-hemolytic species cause oxidation of iron in hemoglobin molecules within the red blood cells. Alpha-hemolytic streptococci include e.g. Streptococcus pneumoniae and Streptococcus viridans. Beta-hemolytic species cause complete rupture of the red blood cells and include e.g. Lancefield groups A and B, also known as ‘group A strep’ and ‘group B strep’. Streptococcus genus includes overall more than 50 species. Streptococcus bacteria cause a variety of infections in humans, including dental caries, pneumonia, endocarditis, meningitis, respiratory tract infections, urinary tract infections, neonatal meningitis, pharyngitis and/or sepsis.

Streptococcus pneumoniae is a common bacterium causing, i.e. pneumonia, meningitis, bronchitis, acute sinusitis, conjunctivitis, osteomyelitis, endocarditis and/or septic arthritis. The bacteria is transmitted by direct contact or via the respiratory route. The bacteria resides in the nasopharynx of healthy carriers and proceeds into an infection under certain circumstances. The infection may be prevented by vaccines, e.g. conjugate vaccine or polysaccharide vaccines. The infection may be treated with antibiotics, e.g. broad-spectrum cephalosporin, and vancomycin, but there is a concern over increasing resistant towards antibodies, According to CDC, Streptococcus pneumoniae is currently resistant to one or more antibiotics in 30% of infections. Streptococcus pneumoniae is resistant to e.g. penicillins. There remains a need for improved, non-antibiotic, therapies for treatment of Streptococcus pneumoniae and other Streptococcus infections. Antibodies for Streptococcus have been developed, as described e.g. in U.S. Pat. No. 5,686,070 and US Patent publication US20070003561, the contents of each of which are herein incorporated by reference in their entirety.

In some embodiments, methods of the present disclosure may be used to prevent, manage and/or treat infections and complications caused by Streptococcus pneumoniae and other Streptococcus bacteria.

Viral particles and methods of using the viral particles described in the present disclosure may be used to prevent, manage and/or treat Streptococcus pneumoniae. As a non-limiting example, the viral particles of the present disclosure comprise a nucleic acid sequence encoding at least one of the sequences described in Table 44.

### Staphylococcus Bacteria

Staphylococcus is a genus of gram-positive bacteria belonging to the family of Staphylococcaceae. The genus includes overall approximately 40 species, Most species of the genus are harmless and reside in the skin and mucous membranes of humans. Staphylococcus bacteria may also be found in the soil. The bacteria may cause diseases either through toxin production or penetration. Staphylococcal toxins are a common cause of food poisoning. Staphylococcus bacteria may cause a variety of diseases, e.g. localized or diffuse skin infection, gastroenteritis, ear infections, septic arthritis, osteomyelitis, sinusitis, infective endocarditis and/or toxic shock syndrome.

Staphylococcus aureus (S. aureus) is typically residing in human nose asymptomatically. In certain circumstances, S. aureus infections may affect many tissues and organs. Individuals with chronic conditions, e.g. diabetes, cancer, vascular disease, eczema and lung disease, have an increased susceptibility towards S. aureus infections. S. aureus may cause skin infections, such as, pimples, impetigo, atopic dermatitis, cellulitis folliculitis. More serious forms of infections include pneumonia, meningitis, osteomyelitis and endocarditis. S. aureus may also cause food poisoning. In severe cases, S. aureus infection may enter the blood stream causing bacteremia and/or sepsis. As of today, there is no medical therapy for prevention of the infection. Some strains of S. aureus may be treated with antibiotics. However, increasing resistance towards antibiotics is a concern. Currently several antibiotic resistant forms of S. aureus exist, including, but not limited to, Methicillin-resistant Staphylococcus aureus (MRSA), Vancomycin-intermediate Staphylococcus aureus (VISA) and Vancomycin-resistant Staphylococcus aureus (VRSA). The drug resistant forms of S. aureus are more frequent in hospital environments.

Staphylococcus epidermidis (S. epidermidis) resides in the normal human skin flora and may cause an infection to individuals with weakened immune system, and to individuals who have catheters, prostheses or surgical implants. S. epidermidis has an ability to colonize on plastic materials or devices placed within the body. The infection may be treated with some antibiotics, but they do not remove the infection and can only be used to manage such infections. Many S. epidermis strains are resistant to antibiotics, such as penicillin, methicillin and/or amoxicillin, and increasing resistance to antibiotics in a concern.

There remains a need for prevention and/or improved treatment therapies against Staphylococcal infections. Antibodies targeting Staphylococcal bacteria have been developed. As an example, pagadaximab (developed by Medlmmune and AstraZeneca) is a monoclonal antibody for prevention of staphylococcal sepsis and may be administered to infants with low birth weight.

In some embodiments, methods of the present disclosure may be used to prevent, manage and/or treat infections and complications caused by Staphylococcus bacteria.

Viral particles and methods of using the viral particles described in the present disclosure may be used to prevent, manage and/or treat Staphylococcal infections. As a non-limiting example, the viral particles of the present disclosure comprise a nucleic acid sequence encoding at least one of the sequences described in Table 45.

Clostridium tetani

Clostridium tetani (C. tetani) is a rod-shaped, anaerobic, Gram-positive bacteria belonging to the family of Clostridiaceae. A matured bacterium develops a terminal spore, which is resistant to heat and common antiseptics. C. tetani produces tetanospasmin toxin. C. tetani is found as spores in soil and in the gastrointestinal tract of animals.

C. tetani infection spreads the tetanospamin toxin to the body, causing tetanus, also known as lock jaw. Tetanus is a dangerous disease characterized by painful tightening of the muscles. The disease may lead to locking of the jaw and neck, leading to inability to open mouth or swallow. The tightening may affect the whole body. In severe cases, the infection may lead to breathing difficulties, pneumonia, or pulmonary embolism. Even more serious is an infection acquired during pregnancy, leading to almost always fatal neonatal tetanus of an infant. The bacteria is typically transmitted through broken skin by direct contact with contaminated soil or objects, or saliva or feces of a contaminated animal. Especially susceptible are individuals with burns, puncture wounds, crush injuries or injuries with dead tissue, individuals having animal bites or scratches. Tetanus is fairly uncommon in developed countries. However, the WHO reported an estimated 50,000 neonatal tetanus deaths in year 2008. A program form elimination of tetanus was started in 1989 by the WHO.

Tetanus may be prevented efficiently by a four vaccine combination, DTaP, Tdap, DT and Td, given to children and adults. For adequate immunity, the primary vaccine is administered during childhood, a booster dose during adolescence and every 10 years thereafter during adulthood. C. tetani infection may be treated with antibiotics, wound care and with human tetanus immune globulin (an antitoxin). Despite the existing treatment methods, approximately 10% of tetanus infections lead to death, according to CDC. There remains a need for longer lasting vaccine as well as improved treatment therapies against C. tetani infections. Antibodies against C. tetani have been developed, as described e.g. by Larrick, J. W. et al., 1992, Immunal. Rev. 130, 69-85, and de Kruif, J. et al., 2009, J. Mol. Biol. 387 (3), 548-558, the contents of each of which are herein incorporated by reference in their entirety.

In some embodiments, methods of the present disclosure may be used to prevent, manage and/or treat infections and complications caused by C. tetani.

Viral particles and methods of using the viral particles described in the present disclosure may be used to prevent, manage and/or treat C. tetani. As a non-limiting example, the viral particles of the present disclosure comprise a nucleic acid sequence encoding at least one of the sequences described in Table 46.

### Bordetella

Bordetella is a genus of Gram-negative, coccobacilli belonging to the family of Alcaliigenaceae. The structure of the bacteria consists of an outer membrane with lipopolysaccharides and phospholipids forming a capsule. Bordetella bacteria affecting humans include, but are not limited to, B. pertussis, B. parapertussis and B. bronchiseptica. B. pertussis resides in the upper air pathways, mostly the trachea and the bronchii, of humans. B. parapertussis resides in the upper air pathways of mammals. The bacteria release toxins that cause damage and swelling of the respiratory pathways.

Pertussis, also known as whooping cough, is a highly contagious infection of the respiratory track caused most commonly by B. pertussis, and occasionally by B. parapertussis. Typical symptoms of the infection include severe coughing and difficulty to breathe accompanied by a runny nose, apnea and fever. Additional complications for infants include pneumonia, convulsions, apnea, and encephalopathy. The bacteria are transmitted through the respiratory tract route. The disease is especially dangerous for infants. According to CDC, about 30,000 infections were reported in the US in 2014. CDC reports 277 deaths occurring from 2000 through 2014, out of which 2-41 where infants less than 3 months of age.

Pertussis may be treated with antibiotics, such as, erythromycin, clarithromycin or azithromycin. However, an increasing resistance to antibiotics is a concern. Pertussis caused B. pertussis may be prevented by vaccination, e.g. by DTaP combination vaccine, which is recommended routinely for infants by CDC and WHO. Despite the widespread vaccination, the disease has insisted. The protection provided by the traditional vaccination is estimated to be 3-6 years. There remains a need for prevention therapies providing a longer lasting immunity, as well as for improved, non-antibiotic, treatments. Antibodies for prevention and/or treatment of pertussis have been developed, as described e.g. in International publication WO2014160098, and Hussein, A. H. et al., 2007, Infect. Immun. 75 (11), 5476-5482, the contents of each of which are herein incorporated by reference in their entirety.

In some embodiments, methods of the present disclosure may be used to prevent, manage and/or treat infections and complications caused by B. pertussis, B. parapertussis and/or other Bordetella bacteria.

Viral particles and methods of using the viral particles described in the present disclosure may be used to prevent, manage and/or treat Bordetella infection. As a non-limiting example, the viral particles of the present disclosure comprise a nucleic acid sequence encoding at least one of the sequences described in Table 47.

### Mycobacterium

Mycobacterium is a genus of nonmotile and aerobic bacteria, belonging to its own family of Mycobacteriaceae. Mycobacteria have an outer membrane, and a hydrophobic and waxy cell wall with mycotic acid/mycolates. The cell wall is neither truly Gram-positive nor-negative. In general, the infections are difficult to treat and the bacteria is naturally resistant to many antibiotics, e.g. penicillin, due to the cell wall. Mycobacteria includes species, such as, but not limited to, M. tuberculosis, Nontuberculous mycobacteria (NTM), M. leprae, M. bovis, M. africanum, and M. microti.

M. tuberculosis is a genetically diverse bacterium and most common and dangerous of the mycobacteria family species. M. tuberculosis causes tuberculosis (TB) which is an infection mainly affecting the lungs. Typical early symptoms include cough, fever, night sweat, and weight loss. The disease may be mild for a period of time and therefore early diagnosis is difficult. Eventually the symptoms get more severe and coughing sputum and blood may occur. TB may be transmitted by the respiratory tract. TB affects all ages of the population, but is most dangerous to children, and individuals with weakened immune systems, e.g. HIV patients. According to the WHO, TB is referred to as a top infectious disease killer worldwide. WHO reports an estimated 9.6 million infections of TB in 2014, out of which 1.5 million cases were fatal. The disease is globally spread, but it is most abundant in the South-East Asia and Western Pacific Regions.

TB may be prevented by vaccinations, i.e. Bacille Clamette-Guerin vaccine. The vaccine is provided for children and adults exposed to environments with high risk of infection. However, the vaccine is not always efficient against TB, e.g. due to the diversity of strains geographically. TB may be treated with a 6 to 9 month course of combinational antimicrobial drug therapy. Antimicrobial drugs effective against TB include e.g. isoniazid, rifampin, ethambutol, and pyrazinamide. However, an increasing resistance towards the medication is a concern. Certain strains of existing TB are identified as multi-drug resistant TB strains, which do not respond to therapy with e.g. isoniazid, rifampicin, or other common drugs. WHO reports an estimated 480 000 multidrug-resistant TB infections in 2014. There remains a need for prevention therapies protecting against broad spectrum of strains, as well as for improved treatment of M. tuberculosis and/or other mycobacteria. Antibodies against mycobacteria have been developed, as described e.g. in US Patent publications US20130309237, US20130309237, US20060229438, the contents of each of which are herein incorporated by reference in their entirety.

In some embodiments, methods of the present disclosure may be used to prevent, manage and/or treat infections and complications caused by tuberculosis and/or other mycobacteria.

Viral particles and methods of using the viral particles described in the present disclosure may be used to prevent, manage and/or treat myobacterium related diseases. As a non-limiting example, the viral particles of the present disclosure comprise a nucleic acid sequence encoding at least one of the sequences described in Table 48.

Francisella tularensis

Francisella tularensis (F. tularensis) is a facultative intracellular Gram-negative, rod-shaped bacterium belonging to the family of Francisellaceae. F. tularensis resides in invertebrates, birds, reptiles, fish, and mammals, including humans. It is one of the most infectious and pathogenic bacteria known (see, e.g. Pechous et al., 2009, Microbial MOl Biol Rev.; 73(4): 684-711).

F. tularensis causes infection called Tularemia. Severity of tularemia varies from mild to fatal. F tularensis may be transmitted to a human by direct skin or eye contact, by the respiratory route or by consumption of contaminated food or drink. Most commonly, the infection is acquired while handling infected animals. Most common form of tularemia is ulceroglandular tularemia, characterized by skin ulcers on the site of infection accompanied by swelling or regional lymph glands. Ulceroglandular tularemia is typically acquire by a tick, or deer fly bite. Pneumonic tularemia is an infection of the respiratory tract characterized by a cough, chest pain, and difficulty of breathing. Pneumonic tularemia is transmitted through the respiratory route and may be fatal if not treated. Oropharyngeal tularemia is transmitted by contaminated food or beverage and causes a sore throat, mouth ulcers, tonsillitis and swelling of lymph glands in the neck. Other forms of tularemia include glandular, oculoglandular (affecting the eyes) and typhoidal (combination of the general symptoms). F. tularensis is considered to be a potential biological and chemical warfare agent.

As of today, there is not preventive therapy for tularemia infection on the market. Some vaccines have been under development (see, e.g. Pechous et al. Microbiol Mol Biol Rev. 2009 December; 73(4): 684-711). Tularemia may be treated with antibiotics, such as, streptomycin, gentamicin, doxycycline, and ciprofloxacin. However, increasing resistance against antibiotics is a concern. There remains a need for improved prevention and treatment therapies for F. tularensis infections. Antibodies against F. tularensis have been developed, e.g. as described by Rynkiewicz, M. J. et al., 2012, Biochemistry, 51 (28), 5684-5694 and Lu, Z., et al., 2013, Immunology, 140 (3), 374-389, the contents of each of which are herein incorporated by reference in their entirety.

In some embodiments, methods of the present disclosure may be used to prevent, manage and/or treat infections and complications caused by F. tularensis.

Viral particles and methods of using the viral particles described in the present disclosure may be used to prevent, manage and/or treat F. tularensis related infections. As a non-limiting example, the viral particles of the present disclosure comprise a nucleic acid sequence encoding at least one of the sequences described in Table 49.

Toxoplasma gondii

Toxoplasma gondii is a parasitic protozoan infecting warm-blooded animals, including humans. Domestic cats and other felines are the most desired hosts for Toxoplasma gondii, as they are the only hosts where the protozoan is capable of sexual reproduction. According to CDC, more than 60 million people in the US may be infected by Toxoplasma gondii.

Toxoplasma gondii causes toxoplasmosis, which is typically asymptomatic in healthy individuals and is controlled by the natural immune system. The infection may be obtained from undercooked, contaminated food, especially pork, lamb and venison, from food contaminated by utensils, or contaminated hands, occasionally from contaminated drinking water, or by the fecal-to-oral route from cat feces. Toxoplasma gondii may also be transmitted by vertical route, especially when the protozoan is acquired during pregnancy. Children infected during or just prior to pregnancy may have eye problems, or brain damage at birth, or may develop symptoms later in their lives. Toxoplasmosis may be dangerous to individuals with a weakened immune system, such as patients with AIDS, undergoing certain chemotherapies or having organ transplants.

Toxoplasmosis may be treated with certain medications such as antibiotics called sulfadiazine and pyrimethamine, which is an anti-parasite medication used for e.g. malaria. However, resistance to both of the medications is an increasing concern. There remains a need for improved treatment methods as well as prevention therapies against Toxoplasma gondii infection. Antibodies targeting Toxoplasma gondii have been developed, as described by e.g. Graille, M. et al., 2005, J. Mol. Biol. 354 (2), 447-458, the contents of which are herein incorporated by reference in their entirety.

In some embodiments, methods of the present disclosure may be used to prevent, manage and/or treat infections and complications caused by Toxoplasma gondii.

Viral particles and methods of using the viral particles described in the present disclosure may be used to prevent, manage and/or treat Toxoplasma gondii related infections. As a non-limiting example, the viral particles of the present disclosure comprise a nucleic acid sequence encoding at least one of the sequences described in Table 51.

### Candida Yeast

Typically, species of yeast are commensals and endosymbionts of human hosts, but may cause an infection under certain circumstances. C. albicans is a yeast belonging to the family of Saccharomycetaceae. C. albicans causes infection of the mouth characterized by white patches on the tongue, mouth and throat. The infection of the mouth is most typical with new born babies, the elderly and individuals with weakened immune system, e.g. HIV/AIDS patients. Optionally, the infection may affect the nails, leading o brittle and defected nails. Optionally, the infection may cause an infection of the vagina, leading to genital burning or uncomfortable discharge. Typically, Candida albicans infections are mild and localized. However, the infection may be severe or fatal for individuals with underlying health problems and left untreated. Invasive candidiasis refers to an infection spreading to many parts of the body, including the heart, brain, eyes, bones and/or joints. Candidemia refers to an infection where candida yeast is present in the blood stream. Severe forms of C. albicans infections affect individuals in health care environments, e.g. patients with central venous catheter, patients treated at an intensive care unit, patients undergoing antibiotic treatments, treatments for kidney failure, recovering from a surgery, and patients with chronic diseases, e.g. diabetes and/or HIV/AIDS. C. albicans is typically transmitted from mother to an infant during childbirth and it remains as a species of human's normal microflora. It may also be transmitted through the sexual transmission route. Other species of candida yeast family include, e.g., C. glabrata, parapsilosis, C. tropicalis, C. krusei and C. lusitaniae.

C. albicans infection may be treated with antifungal drugs, e.g. nystatin, clotrimazole, amphotericin B oral suspension) or systemic oral azoles (e.g. fluconazole, itraconazole, or posaconazole). Despite the medical therapy available, some forms of C. albicans infections are dangerous, or life-threatening. There remains a need for improved prevention, and/or treatment therapies against C. albican infections, for example by antibody therapies. Efungumab (developed by NeuTec Pharma) is an antibody for treatment of invasive C. albicans infection.

In some embodiments, methods of the present disclosure may be used to prevent and/or treat C. albican infections.

Viral particles and methods of using the viral particles described in the present disclosure may be used to prevent, manage and/or treat C. albican related infections. As a non-limiting example, the viral particles of the present disclosure comprise a nucleic acid sequence encoding at least one of the sequences described in Table 52.

### Human Immunodeficiency Virus (HIV)

Human immunodeficiency virus (HIV) is a roughly spherical enveloped RNA virus belonging to the family of Retroviridae. HIV is composed of two positive single-stranded RNA copies. The viral core contains a viral capsule protein, p24, which surrounds the two single stranded RNAs and the enzymes for HIV replication. The viral envelope consists of two lipid layers, the outer layer glycoprotein 120 (gp 120) and the transmembrane glycoprotein 41 (gp41), Gp120 attached to the host cell whereas gp41 has a role in the cell fusion process. For replication, the virus needs a host cell and the RNA first transcribes into DNA by enzyme reverse transcriptase. HIV infects the CD4 lymphocyte (T cell) leading to depletion of CD4+ T cells and loss of CD4+ T-cell function, as infected cell loses its function and converts to a HIV-replicating cell. (see, e.g. Okoye and Picker, 2013, Immunol Rev.; 254(1): 54-64, and references therein). Additionally, HIV infection leads to B lymphocyte (B cell) hyper-activation and dysfunction (see, e.g. Moir and Fauci, 2009, Nat Rev Immunol.; 9(4): 235-245, and references therein). The virus may be transmitted through sexual transmission route, vertical transmission route, iatrogenic (medical procedure) route, or in direct contact with certain body fluids with high concentration of HIV, including e.g. blood, breast milk, semen, vaginal, and rectal secretions. Two types of HIV (HIV-1 and HIV-2) have been identified. HIV-1 has higher infectivity and has spread around the globe whereas HIV-2 is more localized to West Africa. According to CDC, there is about 36.9 million people in the world with HIV/AIDS with about 2 million cases arising every year. The infection is most abundant in Sub-Saharan Africa.

In acute HIV infection stage, within 2-4 weeks after infection, infected patients experience flu-like illness. In the second stage the infection is asymptomatic and the HW replication is at low level. The second stage may last for years or decades, especially when treated with HIV medication. Eventually, HIV causes acquired immune deficiency syndrome (AIDS), which is a clinical condition characterized by severe immunosuppression attacking the CD4 cells, making individuals susceptible to life-threatening malignancies and infections. Complications associated with HIV/AIDS include common bacterial and viral infections, parasite infections, certain cancers (e.g. Kaposi's sarcoma, Non-Hodgkin's lymphoma, and angiosarcoma), progressive multifocal leukoencephalopathy (PN/ft) and wasting.

As of today, there is no prevention therapy or cure for HIV/AIDS. However, with antiretroviral (ART) therapy, the disease may be managed for a long period of time. ART therapy comprises of five classes of drugs used in different combinations to treat HIV. The drugs target the different phases of the retrovirus life-cycle. However, there remains a need for improved therapies for prevention, management and/or treatment of HIV/AIDS.

Antibodies for treatment and prevention of HIV infection have been developed. For example, commercial antibody Ibalizumab (developed by Taimed Biologics Inc.) is a non-immunosuppressive monoclonal antibody binding to CD4, Anaplasma phagocytophilum inhibiting the viral entry process. As another example, suvizumab (developed by Kaktsuden, Chemo-Sero Therapeutic Research Institute) is a humanized antibody targeting the HIV-1 envelope glycoprotein GP120. As a non-limiting example, any of the antibodies in Table 42, variants or fragments thereof may be used in the treatment and/or prevention of HIV.

Antibodies neutralizing HIV-1 and HIV-1 strains have been identified, but as of today, the researchers have not been able to develop a vaccination for HIV. HIV has a capability to evolve with unusually high somatic mutation and recombination rate. So far, conventional vaccines have not succeeded in eliciting analogues of the broadly neutralizing antibodies. An alternative approach suggested involves using adeno-associated vectored gene delivery for expression of antibodies from muscle tissue (e.g. Balasz et al, 2012, Nature Letter, 481, 81-84, Balasz et al, 2014, Nat Med.; 20(3): 296-300, Saunders et al., 2015, J Virol.; 89(16):8334-45, and US Patent publication US20030219733, the contents of which are herein incorporated by reference in their entirety). The studies have demonstrated efficient and long lasting protection from HINT infection by e.g. intravenous or mucosal surface transmission.

Viral Particles and methods of using the viral particles described in the present disclosure may be used to prevent, manage and/or treat HIV infection and AIDS. As a non-limiting example, the viral particles of the present disclosure comprise a nucleic acid sequence encoding at least one of the sequences described in Table 53.

### Therapeutic Applications: Toxins

Viral particles and methods of using the viral particles described in the present disclosure may be used to prevent, manage and/or treat infectious disease. As a non-limiting example, the viral particles of the present disclosure comprise a nucleic acid sequence encoding at least one of the sequences described in Tables 25-28.

Toxins are a group of substances that are highly poisonous and dangerous to humans. Toxins are infectious agents in form of bacteria, viruses, fungi, proteins, and other chemical and/or biological substances. Toxins may lead to fatal conditions. Toxins are produced by nature, and may be produced synthetically. Exposure to toxins may be unintentional and occur when in contact with toxic plants, or contaminated food, water, livestock or animals. Due to the life-threatening impact of toxins, they are considered to be potential biological and/or chemical warfare agents that may be applied as weapons of mass destruction in war field. They also impose a threat to be used as means for terrorist attacks.

### Ricin

Is a naturally occurring carbohydrate-binding lectin protein produced by castor oil plant growing in Eastern Africa, India, Southeastern Mediterranean basin area, and in tropical regions. Ricin may also be manufactured from the waste products when processing castor beans. Ricin has a globular structure with two toxin chains, chain A and chain B, which both need to be present for the cytotoxic affect. Ricin kills cells by inhibiting protein synthesis. Chain B penetrates to the cell whereas the disulfide bond joining chain A to chain B lectin has an affinity to bind to cell surface carbohydrates, (see, e.g. Friedman and Rasooly, 2013, Toxins (Basel); 5(4): 743-775). Ricin is highly toxic to humans with median lethal dose (LD₅₀) of 22 micrograms per kilogram of body weight. The exposure to Ricin may be by inhaling, ingestion or by injection. The symptoms are dependent of the method of exposure. When inhaled, ricing causes severe inflammation of the lungs, causing would has symptoms including coughing, difficulty breathing, muscle ache and nausea. When ingested, ricin induces internal bleeding of the stomach and intestines leading to pain, vomiting and bloody diarrhea, and eventual failure of the kidneys, liver and spleen. When injected, ricin induces failure of the muscles and lymph nodes, and eventually failure of the liver, kidney and spleen. There is no known treatment for Ricin poisoning.

Unintentional poisoning by Ricin is uncommon. However, Ricin is a potential biological and chemical warfare agent creating a need for treatment and prevention therapies for ricin poisoning. Antibodies targeting ricin have been developed, as described e.g. in International publication WO2015100409, the contents of which are herein incorporated by reference in their entirety.

In some embodiments, methods of the present disclosure may be used to prevent, manage and/or treat infections and complications caused by ricin.

Viral particles and methods of using the viral particles described in the present disclosure may be used to prevent, manage and/or treat Ricin related infections and/or conditions. As a non-limiting example, the viral particles of the present disclosure comprise a nucleic acid sequence encoding at least one of the sequences described in Table 25.

Bacillus anthracis

Bacillicus anthracis is a Gram-positive, rod-shaped bacterium causing anthrax disease (see, e.g. Spencer, 2003, J Clin Pathol.; 56(3): 182-187, and references therein). Most animals, especially herbivores, are susceptible to infection of Bacillicus anthracis. Anthrax may be infected via respiratory exposure, skin contact or eating contaminated food, in most cases meat. Inhaled anthrax causes flu-like symptoms, pneumonia and severe respiratory collapse. Gastrointestinal anthrax causes severe diarrhea, acute inflammation of the intestinal tract, and vomiting of blood. Skin exposure to the bacteria will lead to boil-like skin lesions forming an ulcer with black center. Typically, infection to humans occurs by eating contaminated meat or while handling infected animals or their product, such as skin, wool or meat. Bacillicus anthracis is a potential biological warfare agent. In 2001, weeks following the September 11 terrorist attacks, letters containing Bacillicus anthracis were mailed to news media offices and two U.S. Senators resulting in death of five people and infected many more.

Anthrax may be treated with antibiotics, such as penicillin and amoxicillin, and may be prevented by vaccines, developed both for humans and animals. However, due to increased threat of biological warfare and terrorism, improved methods of treatment are in demand. Anthrax may also be treated by antibody therapy. For example, Raxibacumab (developed by Cambridge Antibody Technology and Human Genome Sciences) is an antibody for the prophylaxis and treatment of inhaled anthrax.

In some embodiments, methods of the present disclosure may be used to prevent, manage and/or treat infections and complications caused by Bacillicus anthracis.

Viral particles and methods of using the viral particles described in the present disclosure may be used to prevent, manage and/or treat Bacillicus anthracis related infections and/or conditions. As a non-limiting example, the viral particles of the present disclosure comprise a nucleic acid sequence encoding at least one of the sequences described in Table 26.

### Shiga Toxin and Shiga-Like Toxin

Shiga toxin, including two major types Stx1 and Stx2, is a toxin produced by Shigella dysenteriae, a rod-shaped bacteria belonging to bacterial genus Shigella. Shiga toxin inhibits protein synthesis within cells. The toxin enters cell via a marcopinosome and inhibits the protein synthesis by cleaving a specific nucleobase RNA of the 60S subunit of ribosome. Shiga-like toxins 1 and 2 are structurally similar to Stx1 and Stx2 and are produced by enterohemorrhagic strains of Escherichia coli (EHEC) strains. (see, e.g. Friedman and Rasooly, Toxins (Basel). 2013 April; 5(4): 743-775). EHEC type 0157 is the most common pathogen causing E. Coli outbreaks in the US. Stx2 is considered to be orders of magnitude more toxic that Stx1. The severity of Shiga toxin foodborne illnesses range from mild diarrhea to a life-threatening complication known as hemolytic uremic syndrome (HUS). HUS is a disease associated with hemolytic anemia, acute kidney failure and low platelet count. Cattle is the major source or infection to humans, but the disease may be spread by birds or pigs. Shiga infection is typically obtained from contaminated food or drink, such as meat, unpasteurized milk, or contaminated water, or by contact with cattle. Shiga toxin and Shiga-like toxins considered to be potential chemical and biological warfare agents.

As of today, there is no prevention therapy or specific treatment for Shiga and Shiga-like toxins. Recent developments have been made in antibody therapy of Shiga toxin induced HUS. For example, SHIGAMAB™ (developed by Bellus Health Inc.) is a monoclonal antibody for treatment of Shiga toxin induced HUS.

In some embodiments, methods of the present disclosure may be used to prevent, manage and/or treat infections and complications caused by Shigella dysenteriae.

Viral particles and methods of using the viral particles described in the present disclosure may be used to prevent, manage and/or treat Shigella dysenteriae related infections and/or conditions. As a non-limiting example, the viral particles of the present disclosure comprise a nucleic acid sequence encoding at least one of the sequences described in Table 28.

### Botulinum Toxins

Botulinum toxins are neurotoxins produced by Clostridium bacteria and they cause a disease called botulism which is characterized by weakness, problems in vision, tiredness, and problems with speech, followed by weakness of the arms, chest muscles and legs. Botulism may be fatal. There are seven different botulinum neurotoxins with a four-domain structure varying in antigenic properties and interactions with intracellular targets. L-chain enters the cytosol, cleaves the synaptosomal protein and blocks neurotransmitter release resulting in peripheral neuromuscular blockade and flaccid paralysis in humans. (see, e.g. Friedman and Rasooly, Toxins (Basel). 2013 April; 5(4): 743-775) Botulinum neurotoxins are highly dangerous to humans, serotype A having a median lethal dose (LD₅₀) of 0.8 micrograms for a human of 70 kg weight. The bacteria is common in soil and water and may produce the botulinum toxins when exposed to low oxygen levels and certain temperatures. Outbreaks of foodborne botulism occur occasionally. Most susceptible to contamination by botulinum are baked products, fresh mussels, canned fruit and vegetables. Infant botulism occurs when the toxins are produced and released by bacteria in the infant's intestines. Botulism may also occur in wounds where the bacteria in the absence of oxygen produces and releases the toxins. Wound botulism is most common in cases where contaminated needles are used for injection. Botulinum toxins are potential biological and chemical warfare agents.

As of today, there is no prevention therapy for botulism. Botulism may be treated with antitoxins that block the circulation of toxins in the blood and prevent worsening of the disease. However, the antitoxins are expensive and not easily available. In cases of wound botulism, the area infected may be removed surgically. Additionally, good supportive care therapy is applied. There remains a need for therapies to prevent and treat botulism. Antibodies targeting botulinum toxins are developed, as described e.g. in US Patent publication US20130058962, and International publication WO2015100409, the contents of each of which are herein incorporated by reference in their entirety.

In some embodiments, methods of the present disclosure may be used to prevent, manage and/or treat infections and complications caused by botulinum toxins.

Viral particles and methods of using the viral particles described in the present disclosure may be used to prevent, manage and/or treat botulinum toxin related infections and/or conditions. As a non-limiting example, the viral particles of the present disclosure comprise a nucleic acid sequence encoding at least one of the sequences described in Table 27.

### Therapeutic Applications: Neglected Tropical Diseases (NTDs)

Viral particles and methods of using the viral particles described in the present disclosure may be used to prevent, manage and/or treat infectious disease. As a non-limiting example, the viral particles of the present disclosure comprise a nucleic acid sequence encoding at least one of the sequences described in Tables 21-24.

Neglected Tropical diseases (NTDs) are a diverse category of communicable diseases present in tropical and subtropical environments. NTDs affect more than one billion people in about 150 countries. NTDs are a significant public health problem costing the involved developing economies billions of dollars annually. The diseases affect mostly the populations with inadequate sanitation, and those in contact with infectious vectors, domestic animals and livestock. In May 2013, the 66th WHO Assembly announced resolution WHA66.12 to integrate measures and plan investments to improve the wellbeing of populations affected by NTDs. NTDs include Buruli ulcer, Chagas disease, Dengue and Chikungunya, Dracunculiasis (guinea-worm disease), Echinococcosis, Endemic treponematoses (Yaws), Foodborne trematodiases, Human African trypanosomiasis (sleeping sickness), Leishmaniasis, Leprosy (Hansen disease), Lymphatic filariasis, Onchocerciasis (river blindness), Rabies, Schistosomiasis, Soil-transmitted helminthiaces, Taeniasis/Cysticercosis and Trachoma.

### Chikungunya Virus

Chikungunya virus is an arbovirus belonging to the Togoviridae family. The genome is a single-strand RNA molecule encoding four non-structural and three structural glycoproteins (C, E1, E2) (see, e.g. Caglioti et at, 2013, New Microbiol; 36(4211-27, and references therein). Chikungunya fever is a mosquito-borne disease caused by chikungunya virus. The symptoms include a fever lasting 2-7 days, rash and flu-like symptoms accompanied by a joint pain that may last for weeks, months or even years. The disease may be dangerous for the elderly and individuals with chronic medical problems. Chikungunya virus is spread by Aedes albopictus and Aedes aegypti. Outbreaks of chikungunya fever have occurred in Africa, Asia, Europe and Indian and Pacific Oceans, and more recently in islands in the Caribbean. As an example, according to the WHO, an outbreak of 1.9 million cases in India, Indonesia, Maldives, Myanmar and Thailand since 2005 has been reported. More recently, as of April 2015 more than million cases have reported in Caribbean Islands, Latin American countries and the United States.

As of today, there is no specific treatment or vaccination for chikungunya fever. The disease is typically treated with supportive care therapy, as well as anti-inflammatory drugs and medicines to relieve the symptoms. Research and development on vaccinations has been done but none has been approved for commercial use so far. Antibodies for detection and treatment of Chikungunya have been developed. E.g, fully human antibodies binding to an epitope located in an antigenic site of the chikungunya virus E1 and E2 envelope proteins were in US Patent Publication US20130189279, the contents of each of which are incorporated herein by reference in their entirety.

In some embodiments, methods of the present disclosure may be used to prevent, manage and/or treat infections and complications caused by chikungunya virus.

Viral particles and methods of using the viral particles described in the present disclosure may be used to prevent, manage and/or treat chikunguyna virus related infections and/or conditions. As a non-limiting example, the viral particles of the present disclosure comprise a nucleic acid sequence encoding at least one of the sequences described in Table 24.

### Dengue Virus

Dengue virus belongs to the family of Flaviviridae, genus of Flavivirus. It is an enveloped, positive strand RNA virus containing two integral membrane proteins envelope (E) and premembrane (prM). Dengue virus is closely related to e.g. Yellow fever, West Nile virus and St. Louis and Japanese encephalitis viruses. There are five serotypes of the virus that can cause dengue fever, which is a mosquito-borne tropical disease. Neutralizing antibodies target the protein E as it binds to the cellular receptors and mediates the viral entry into cells. Infection with a serotype may produce a lifelong immunity to that serotype but no long-term immunity against other serotypes, (see e.g., Wahala. and de Silva, 2011, Viruses.; 3(12): 2374-2395, and references therein). In fact, an infection by a second serotype may lead to a more severe form of disease, due to the complexity of the antibody respond and possible antibody dependent enhancement (ADE), which hypothesizes that weakly neutralizing antibodies from the first infection bind to the second serotype and enhance the infection. The symptoms of dengue fever are similar to flu, including fever, headache, muscle and joint pain and skin rash. The disease may also manifest as a potentially lethal complication called severe dengue, also known as dengue hemorrhagic fever. The disease may be dangerous to individuals with chronic diseases, such as diabetes or asthma, or children and the elderly. Dengue virus is spread by several mosquito species, out of which Aedes aegypti is the most common. Dengue may also be transmitted via infected blood or organ donation or by the vertical transmission route. According to the WHO, the estimated number of dengue infections annually could be as high as 390 million.

As of today, there is no specific treatment or prevention therapy for dengue fever. Antibodies targeting dengue virus have been developed. As an example, antibodies neutralizing four serotypes of dengue virus have been in US Patent publication US20150225474, US20150218255 and in U.S. Pat. No. 9,073,981 the contents of each of which are incorporated herein by reference in their entirety.

In some embodiments, methods of the present disclosure may be used to prevent, manage and/or treat infections and complications caused by dengue virus.

Viral particles and methods of using the viral particles described in the present disclosure may be used to prevent, manage and/or treat Dengue virus related infections and/or conditions. As a non-limiting example, the viral particles of the present disclosure comprise a nucleic acid sequence encoding at least one of the sequences described in Table 21.

Trypanosoma cruzi

Trypanosoma Cruzi (T. cruzi) is a species of parasitic euglenoid protozoan. T. cruzi causes Chagas disease, also known as American trypanosomiasis, which is a tropical parasitic disease. The symptoms of Chagas disease at the early stage include fever, swollen lymph nodes, headaches or local swelling at the site of bite. The chronic phase of Chagas starts after 8-12 weeks, which may be symptomless, or include enlargement of the ventricles of the heart, which may result in heart failure, or to an enlarged esophagus or enlarged colon. The severity of Chagas disease varies from almost unnoticeable to fatal. Chagas disease is spread by an insect vector triatomine bug. These bugs get infected with T. cruzi by feeding on the blood of an infected human or animals, and they spread it further by bites and ingestion of blood. The triatomine bug is also known as a “kissing bug” referring to its tendency to feed on people's faces. T. cruzi may also be transmitted through blood transfusions or through breast milk. Chagas disease is present mainly in 12 Latin American countries but has also spread to other continents. According The WHO, over 10 000 people die every year from Chagas disease, and 25 million people are in the risk of infection.

As of today, there is no specific prevention or treatment therapy for Chagas disease. The traditional therapies for Chagas have been involved with attempts to kill the parasite and treatment of the symptoms. For example, azole and nitro-derivative drugs have been used, but have not been successful in removal of the parasite fully. Other mechanisms to treat the disease have been under research. After infection in mammals, the parasite incorporates a charged carbohydrate (sialic acid) to survive to the chronic phase of the disease. To do so, the parasite scavenged sialic acid it from the host's sialoglycoconjugates, through a transglycosylation reaction catalyzed by an enzyme called trans-sialidase. The trans-sialidase has been identified as a potential target for drug development. Buschiazzo et al. have reported an antibody inhibiting the T. cruzi trans-sialidase enzyme providing an antibody therapy mechanism for Chagas disease (see, Buschiazzo et al., 2012, PLoS Pathol. 8 (1), E1002474, and references therein).

In some embodiments, methods of the present disclosure may be used to prevent, manage and/or treat infections and complications caused by Chagas disease.

Viral particles and methods of using the viral particles described in the present disclosure may be used to prevent, manage and/or treat Chagas disease. As a non-limiting example, the viral particles of the present disclosure comprise a nucleic acid sequence encoding at least one of the sequences described in Table 23.

### Rabies Virus

Lyssaviruses are a genus of RNA viruses belonging to the family of Rhabdoviridae. Rabies virus is a neurotropic virus with cylindrical morphology. After infection, rabies virus enters the peripheral nervous system, and further to central nervous system by retrograde axonal transport. Rabies virus and Australian bat lyssavirus cause rabies. Rabies affects humans and warm-blooded animals. The early stage symptoms include flu-like signs, but later the disease manifests as paralysis, anxiety, insomnia, abnormal behavior, hallucinations. Humans and animals infected may also experience hydrophobia, “fear of water”, which is considered a characteristic symptom of the disease. Eventually the disease affects the central nervous system and brain, causing death. Humans are typically infected by being bitten, scratched or licked by an animal with the disease. Most commonly the infection is by dogs. Whereas efficient vaccination programs for animals have been able to reduce or even eliminate rabies in developing countries, the disease still affects poor population mainly in Africa and Asia. According to the WHO, post-bite treatment and vaccination is provided for 15 million people annually.

Rabies is a vaccine-preventable disease and especially systematic vaccination of dogs has been a cost-effective strategy for prevention of rabies. Post-exposure prophylaxis (PEP), the treatment of bite victims immediately after the exposure, includes local treatment of the wound, rabies vaccination and administration of rabies immunoglobulin. Though efficient vaccines for rabies have been developed, there remains a need for treatment/or management of rabies to prevent death after rabies virus has entered the central nervous system (see, e.g., Hicks et al., 2012, Clin Exp Immunol., 169(3): 199-204, and references therein). The genome of rabies virus codes for five viral proteins. Out of the five, G protein, which is an external surface glycoprotein, forms protrusions that cover the outer surface of the virion envelope and is known to induce neutralizing antibodies. Also, nucleoprotein (N) molecules and the phospho-protein (NS) participate in immune responses. G protein has been the target of antibody developments. For example, therapeutic antibodies against rabies virus are taught in U.S. Pat. Nos. 7,071,319, 6,890,532, and 9,005,624, the contents of each of which are incorporated herein by reference in their entirety.

In some embodiments, methods of the present disclosure may be used to prevent, manage and/or treat infections and complications caused by rabies virus.

Viral particles and methods of using the viral particles described in the present disclosure may be used to prevent, manage and/or treat rabies virus related infections and/or conditions. As a non-limiting example, the viral particles of the present disclosure comprise a nucleic acid sequence encoding at least one of the sequences described in Table 22.

### Therapeutic Applications: Tropical Diseases (TDs) and Vector-Borne Diseases

Viral particles and methods of using the viral particles described in the present disclosure may be used to prevent, manage and/or treat infectious disease. As a non-limiting example, the viral particles of the present disclosure comprise a nucleic acid sequence encoding at least one of the sequences described in Tables 29-31.

Plasmodium falciparum

Plasmodium falciparum (P. falciparum) is a protozoan parasite belonging to Plasmodium parasite family. P. falciparum is the main cause of malaria and responsible for nearly all death cases in malaria. P. falciparum is released to the human bloodstream through mosquito saliva. The parasite has a high rate of replication and capability to alter. P, falciparum, among other Plasmodium parasites, cause malaria, which is a mosquito borne tropical disease. The early stage symptoms include fever, headache, chills and vomiting. If not treated at the early stage, malaria can progress to a life-threatening condition involving multiple organs, resulting in skin yellowing, seizures and coma. In children, malaria may cause severe anemia, respiratory distress in relation to metabolic acidosis, and/or cerebral malaria. The disease is especially dangerous for young children, pregnant women and individuals without immunity to the disease, such as travelers from non-malaria areas. An infection may develop a partial immunity, allowing the following infections to be asymptomatic. According to the WHO, about half of world's population are at risk of malaria. Sub-Saharan Africa carries the highest density of malaria. In 2015, 88% of malaria cases and 90% of malaria deaths was in Sub-Saharan Africa. Malaria is spread by female Anopheles mosquitos and caused by 5 different parasite species, out of which Plasmodium falciparum is the most prevalent and responsible for the severe cases of malaria.

Despite tremendous efforts, there is no commercial vaccination for malaria. Traditional treatment for malaria consists of antimalarial medicine therapies, such as artemisinin-based combination therapies, which consists of artemisinin combined with antimalarial drugs such as amodiaquine, lumefantrine, mefloquine and sulfadoxine/pyrimethamine. However, drug resistance has been a serious challenge in malaria treatment. Currently resistance is common for all antimalarial medications apart from artemisinin combination therapy. The cost of artemisinin treatment is high and there remains a need for prevention therapies and improved treatment against malaria.

Due to the polymorphic nature and high replication rate of P Falciparum, tolerance to malaria is achieved only after years of repeated infections. Antibodies for prevention and treatment of malaria have been developed. For example, antibodies against P. falciparum are taught in U.S. Pat. No. 7,811,569, in US Patent publication US20150197562 and in international Patent publication WO2014087007, the contents of each of which are incorporated herein by reference in their entirety. A need for mechanism to deliver constant, effective concentration of malaria antibody for a long period is still in need. Studies by Deal et al. demonstrate results on vectored immunoprophylaxis delivery of malaria antibodies to mice (see, Deal et al. PNAS, 2014, 111(34), 12528-12532).

In some embodiments, methods of the present disclosure may be used to prevent, manage and/or treat infections and complications caused by P. falciparum.

Viral particles and methods of using the viral particles described in the present disclosure may be used to prevent, manage and/or treat P. falciparum related infections and/or conditions. As a non-limiting example, the viral particles of the present disclosure comprise a nucleic acid sequence encoding at least one of the sequences described in Table 29.

### Ebola Virus

Genus of Ebola virus includes five viruses, Zaire, Reston, Sudan, Tai Forest and Bundibygyo Ebola viruses, is a negative-sense RNA virus belonging to the family of filoviridae. The West Africa outbreak has been associated with Zaire Ebola virus. The genome of Ebolavirus encodes seven genes. The glycoprotein GP gene encodes two distinct gene products: sGP which is a dimeric and secreted glycoprotein and less abundant GP, which is a trimeric-virion attached, membrane embedded envelope glycoprotein and responsible for the virus attachment, fusion and entry during infection. Ebola virus disease is a hemorrhagic fever disease caused. The early symptoms include fever, sore throat, muscular pain, followed by a diarrhea and rash. Eventually the disease will affect the liver and kidney function and cause internal bleeding. The disease is highly fatal, as about 50% infected individuals die. The Ebola virus is transmitted by direct contact with the blood and body fluids and tissues of an infected person or an animal, most commonly a chimpanzee, gorilla, fruit bat, monkey, forest antelope and porcupine. The disease is also transmitted when handling dead bodies of infected animals or humans. Also, sexual transmittance of the disease has been suggested. The WHO has reported more than 28 000 infections and 11 000 deaths in Ebola virus disease outbreak in West Africa (2014-present), mainly affecting Guinea, Sierra Leone and Liberia.

As of today, there is no licensed treatment or prevention therapy proven to neutralize the virus, Typically, Ebola virus disease is treated with a good supportive care. A variety of blood, immunological and drug therapies are under investigation, as well as preventive vaccines undergoing evaluations. However, a demand for effective therapies for treatment and prevention of Ebola virus disease remain.

Viral surface of GP has been identified as a target for neutralizing antibodies. Antibodies targeting GP of Ebola virus have been taught, e.g. in International Patent publication WO2015127136 and Olal, D., et al., 2012, J. Tirol. 86 (5), 2809-2816, the contents of each of which are incorporated herein by reference in their entirety.

In some embodiments, methods of the present disclosure may be used to prevent, manage and/or treat infections and complications caused by Ebola virus.

Viral particles and methods of using the viral particles described in the present disclosure may be used to prevent, manage and/or treat Ebola related infections and/or conditions. As a non-limiting example, the viral particles of the present disclosure comprise a nucleic acid sequence encoding at least one of the sequences described in Table 30.

### Marburg Virus

Marburg virus belongs to the filoviridae family of viruses with coiled, toroid or branched structures with seven proteins. The structure of Marburg virus is similar to Ebola virus, however, the involved antigens are different. The filoviruses express a single glycoprotein on their surface. The glycoprotein is responsible for the infection, as it is involved in the attachment and entry of the viruses causing infection. Marburg virus disease is a hemorrhaging fever disease caused by Marburg virus. It is highly fatal disease and related to Ebola virus diseases. The early symptoms of the disease include severe headache and malaise. Severe hemorrhagic manifestations in later stages include bleeding from multiple sites. The Marburg virus is transmitted by direct contact with the blood and body fluids and tissues of infected persons or animals, most commonly fruit bats and monkeys. The disease is also transmitted when handling dead the bodies of infected animals or humans. Marburg virus disease is uncommon, but outbreaks typically have a high rate of fatality. According to the WHO, the death rate was as high 80% in outbreaks of 1998-2000 in Democratic Republic of Congo and 2005 in Angola.

As of today, there is no preventive or treatment therapy for Marburg virus disease. The current treatment methods include good supportive treatment. The surface glycoprotein has been a target for development of antibodies for Marburg disease vaccines and treatments. For example, International Patent publication WO2015127140, and US Patent publication US20140356354, the contents of which are incorporated herein by reference in their entirety, teach therapeutic antibodies that recognize glycoprotein of filoviruses for different strains of Marburg, as well as Ebola.

In some embodiments, methods of the present disclosure may be used to prevent, manage and/or treat infections and complications caused by Marburg virus.

Viral particles and methods of using the viral particles described in the present disclosure may be used to prevent, manage and/or treat Marburg related infections and/or conditions. As a non-limiting example, the viral particles of the present disclosure comprise a nucleic acid sequence encoding at least one of the sequences described in Table 30.

### West Nile Virus

West Nile virus (WNV) is a positive-stranded RNA of the flavivirus genome and member of the Japanese encephalitis serocomplex of flaviviruses, (see Throsby, M., J. Virol. 80 (14), 6982-6992 (2006)). Two lineages of the virus have been identified. The genome of the virus encodes a single polyprotein producing three structural proteins, capsid C, precursor membrane prM and envelope E as well as seven nonstructural proteins. WNV causes mosquito-borne infections with a variety of manifestations. Tough about 80% of WNV infections are symptomless and not harmful, in certain cases, the disease may lead to fatal neurological diseases. Infection of MNV may lead to a West Nile fever, which causes flu-like symptoms accompanied by high fever, headache, chills, excessive sweating, fatigue, weakness, swollen lymph nodes, and joint pains. Infection by MNV may also occur as cutaneous manifestations, including rashes that may include punctate erythematous, macular and popular eruptions. West Nile infections may also affect the central nervous system resulting in West Nile neuroinvasive diseases, including meningitis, encephalitis, meningoencephalitis and poliomyelitis-like syndrome. These neuroinvasive forms of NWV infections occur in only about 1% of infections, but they may be life-threatening. WNV is commonly found in Africa, Europe, the Middle East, North America and West Asia. WNV is typically transmitted to humans and other mammals by mosquitos and is maintained in nature in a cycle involving transmission between birds and mosquitoes. WNV is carried by different types of mosquitos, dependent on geographical distribution. Transmission to humans may also occur from birds, horses or other humans.

As of today, there is no specific treatment or prevention therapy for MNV infections. Current methods of treatment include good supportive care. Due to severity of some of the manifestations, there remains a need for such therapies. Envelope E has been a target of most antibody related studies. Antibodies targeting M and the first non-structural protein have also been investigated. As an example, Thorsby et al., 2006, J. Virol. 80 (14), 6982-6992, the contents of which are incorporated herein by reference in their entirety, teaches antibodies binding to E and prM proteins. U.S. Pat. Nos. 8,663,950 and 7,527,973, the contents of each of which are incorporated herein by reference in their entirety, teach antibodies binding to E protein of WNV.

In some embodiments, methods of the present disclosure may be used to prevent, manage and/or treat infections and complications caused by West Nile virus.

Viral particles and methods of using the viral particles described in the present disclosure may be used to prevent, manage and/or treat West Nile virus related infections and/or conditions. As a non-limiting example, the viral particles of the present disclosure comprise a nucleic acid sequence encoding at least one of the sequences described in Table 31.

### Yellow Fever Virus

Yellow fever virus is an enveloped RNA virus belonging to the Flavivirus family. Yellow fever, also known as Yellow Jack, Yellow Plague or Bronze John, is a mosquito-borne viral hemorrhagic disease. In most cases, the symptoms include fever, headache, chills, loss of appetite, nausea, and muscle pain. In some occasions, the disease progresses to a second stage which includes fever accompanied by abdominal pains, liver damage resulting in jaundice, kidney problems and/or bleeding. The disease is spread primarily by Aedes and Haemogogus type mosquitos. The disease is most typical in tropical environments. According to the WHO, there are 200 000 annual cases of yellow fever resulting in 30 000 deaths mainly in Africa and Latin America. 90% of cases occur in Africa.

Preventive live-attenuated vaccines for yellow fever are available. However, concern related to post-vaccine adverse events has decreased the popularity of the vaccines. The vaccination is not recommended to infants younger than 9 months, pregnant women and individuals with an immune deficiency. As of today, there is no specific treatment for yellow fever. Current methods for treatment involve with supportive care to treat dehydration, respiratory failure and fever. There is a need for improved prevention and treatment therapies against yellow fever virus.

Envelope E glycoprotein of yellow fever virus has been identified as a potential target for antibody therapies. Neutralizing antibodies for yellow fever virus have been reported by Thibodeaux, B. A. et al, 2012, Antiviral Res. 94 (1), 1-8 and Daffis, S. et al., 2005, Virology, 337 (2), 262-272, the contents of each of which are incorporated herein by reference in their entirety.

In some embodiments, methods of the present disclosure may be used to prevent, manage and/or treat infections and complications caused by yellow fever virus.

Viral particles and methods of using the viral particles described in the present disclosure may be used to prevent, manage and/or treat yellow fever virus related infections and/or conditions. As a non-limiting example, the viral particles of the present disclosure comprise a nucleic acid sequence encoding at least one of the sequences described in Table 31.

### Japanese Encephalitis Virus

Japanese encephalitis virus is an enveloped positive sense single-stranded RNA virus belonging to Flavivirus family and closely related to St. Louis encephalitis and West Nile virus. The virus causes Japanese encephalitis, also known as Japanese B encephalitis. In majority of cases, the disease is symptomless. However, in less than 1% of infections, the disease leads to a life-threatening encephalitis. The early stage symptoms include fever, headache and malaise. As the disease progresses into an acute encephalitis, the symptoms include neck rigidity, cachexia, hemiparesis, convulsions and fever, accompanied by lifelong neurological problems such as deafness, and/or mental retardation. The disease is transmitted to humans via mosquitos of the Culex species. The virus exists in a transmission cycle between mosquitos, pigs, and water birds. The disease affects 24 counties in the South-East Asia and Western Pacific. According to the WHO, an estimated 68 000 clinical cases are reported annually, with case-fatality rate as high as 30%. Major outbreaks of the disease occur every 2-15 years.

The disease may be prevented by a vaccination, most common vaccination being a live attenuated vaccine. In general, the vaccines initially show high effectiveness, but the protection decreases over time. As of today, there is no specific treatment for the disease. Current treatment therapies include good supportive care. There remains a need for longer lasting, improved prevention therapies, and treatment for Japanese encephalitis virus infections.

Antibodies for treatment of Japanese encephalitis have been developed. For example, Hsieh et al. teach antibodies that target cellular receptors and interrupts their function in flavivirus infections in US Patent publication US20080292644, the contents of which are incorporated herein by reference in their entirety.

In some embodiments, methods of the present disclosure may be used to prevent, manage and/or treat infections and complications caused by Japanese encephalitis virus.

Viral particles and methods of using the viral particles described in the present disclosure may be used to prevent, manage and/or treat Japanese encephalitis virus related infections and/or conditions. As a non-limiting example, the viral particles of the present disclosure comprise a nucleic acid sequence encoding at least one of the sequences described in Table 31.

St. Louis Encephalitis Virus

St. Louis encephalitis virus is a positive-stranded RNA virus and member of the Flavivirus family and closely related to Japanese encephalitis virus. St. Louis encephalitis is a mosquito-borne disease caused by the virus. In majority of cases, the disease is symptomless. However, in less than 1% of the cases, the disease may lead to encephalitis, which may be life-threatening, especially for the elderly. The early stage symptoms include fever, headache, dizziness, malaise and nausea. If the disease progresses to the central nervous system, symptoms include stiff neck, confusion, disorientation, dizziness, tremor and unsteadiness, and in severe cases coma or even death. St. Louis encephalitis virus is transmitted to humans through Culex mosquitos. The virus exists in a transmission cycle between mosquitos and birds. The disease mainly affects the USA, especially eastern and central states. The disease has also spread to Canada and Mexico.

As of today, there is no vaccine or specific treatment for St. Louis encephalitis. Current treatment therapies include good supportive care. There is a demand for preventive and treatment therapies for the disease. Neutralizing antibodies for St. Louis encephalitis virus have been reported in Thibodeaux, B. A., et al, 2012, Antiviral Res, 94 (1), 1-8 and Daffis, S, et al., 2005, Virology 337 (2), 262-272, the contents of which are incorporated herein by reference in their entirety.

In some embodiments, methods of the present disclosure may be used to prevent, manage and/or treat infections and complications caused by St. Louis encephalitis virus.

Viral particles and methods of using the viral particles described in the present disclosure may be used to prevent, manage and/or treat St. Louis encephalitis virus related infections and/or conditions. As a non-limiting example, the viral particles of the present disclosure comprise a nucleic acid sequence encoding at least one of the sequences described in Table 31.

### Therapeutic Applications: Foodborne Illness and Gastroenteritis

Viral particles and methods of using the viral particles described in the present disclosure may be used to prevent, manage and/or treat infectious disease. As a non-limiting example, the viral particles of the present disclosure comprise a nucleic acid sequence encoding at least one of the sequences described in Tables 14-20.

Foodborne illnesses, also known as food poisoning, are a common and costly public health problem. The illnesses are typically transmitted by the fecal-oral-route. The transmission to humans is by consuming contaminated food or beverage. More than 250 different foodborne diseases, mostly infections caused by viruses, bacteria, parasites or fungus, are identified by the CDC. CDC estimates that approximately 48 million individuals are affected by foodborne illnesses annually in the United States. Gastroenteritis is an inflammation of the gastrointestinal tract involving stomach and small intestine. Gastroenteritis is also caused by an infection caused by viruses, bacteria, parasites or fungus. The transmission to humans is by person-to-person contact, or by consuming contaminated food or beverage. Foodborne illnesses and gastroenteritis have similar symptoms including diarrhea, vomiting, abdominal pain, dehydration. In some cases, the diseases may require hospitalization or be fatal. Both illnesses are best prevented by proper hand hygiene, proper hygiene while preparing food, treatments to kill bacteria such as pasteurizing, cooking or heating food, and proper methods to store food.

### Rotavirus

Rotavirus is a double-stranded RNA virus belonging to the family of Reoviridae. The rotavirus genome consists of 10 segments coding for a single protein, and segment 11 coding for two proteins. The virions are non-enveloped, triple-layered and icosahedral in structure (see, e.g. Aiyegbo et al., 2013, Pleas One 8, 61101, and references therein). The virus is spread by the fecal-oral-route. Rotavirus is very common especially among infants and young children and spreads easily. Almost all children worldwide are infected with rotavirus by the age of 5, and the disease leads to death of half a million children annually. Rotavirus causes rotavirus gastroenteritis with symptoms including nausea, vomiting, diarrhea and fever. Rotavirus is associated with dehydration. The disease is milder in adults and more severe in young children, infants and the elderly. Though infection does not provide full immunity to the virus, the first infection is typically the most severe in symptoms.

As of today, there is no specific treatment rotavirus infections. Present treatment includes good supportive care including drinking of fluids to prevent dehydration. In severe cases, the rotavirus gastroenteritis requires hospital care e.g. treatment with intravenous fluids. Vaccines for prevention of the disease have been developed and CDC recommends rotavirus vaccination for infants as part of the routine vaccinations. There remains a need for medical treatment therapies for the infection. Development has been done in the field of antibodies. E.g. Aiyegbo et al., in plus One 8, 61101 (2013, teach antibodies targeting the intermediate capsid layer of Is/P6 of the triple-layered particle and Frenken et al. teach anti-rotavirus antibodies in U.S. Pat. No. 8,105,592, the contents of which are incorporated herein by reference in their entirety.

In some embodiments, methods of the present disclosure may be used to prevent, manage and/or treat infections and complications caused by rotavirus.

Viral particles and methods of using the viral particles described in the present disclosure may be used to prevent, manage and/or treat rotavirus related infections and/or conditions. As a non-limiting example, the viral particles of the present disclosure comprise a nucleic acid sequence encoding at least one of the sequences described in Table 19.

### Norwalk Virus/Norovirus

Norwalk virus, also known as winter vomiting bug, is the only member of genus norovirus belonging to the family of Caliciviridae. Norwalk virus is a single-stranded RNA with three open-reading frames that encode a polyprotein precursor to non-structural proteins, and two polypeptides of different sizes (see e.g. Jiang et al., 1993, Virology; 195(1):51-61, and references therein). Norwalk virus is spread by the fecal-oral-route. Norwalk virus is extremely contagious and can be transmitted through contaminated food or drink, touching contaminated surfaces or Objects or from a contact with an infected individual. The Norwalk virus causes an inflammation of stomach and/or intestines. The symptoms associated with the infection include stomach pain, nausea, vomiting and diarrhea. The disease can be dangerous, especially for your children or young adults. According to CDC, every year 1921 million infections occur leading to 570-800 deaths in the US.

As of today, there is no vaccine or specific treatment for Norwalk virus associated gastroenteritis. Antibodies for prevention and treatment of Norwalk virus have been developed. For example, International Patent publication WO2014126921 and WO2014183052, the contents of each of which are incorporated herein by reference in their entirety, teach neutralizing antibodies binding to the polypeptides of Norwalk virus.

In some embodiments, methods of the present disclosure may be used to prevent, manage and/or treat infections and complications caused by Norwalk virus.

Viral particles and methods of using the viral particles described in the present disclosure may be used to prevent, manage and/or treat Norwalk virus related infections and/or conditions. As a non-limiting example, the viral particles of the present disclosure comprise a nucleic acid sequence encoding at least one of the sequences described in Table 18.

Campylobacter jejuni

Campylobacter jejuni (C. jejuni) is an oxidase-positive, catalase-positive, nonfermentative Gram-negative bacteria with a helical shape. The C. jejuni inhabits in the intestinal tract of animals (e.g. poultry, cattle, pigs, sheep, ostriches and shellfish), and in pets (e.g, cats and dogs). The bacteria may be transmitted to humans foodborne, e.g. when eating contaminated food or drink, such as unpasteurized milk. According to the WHO, campylobacter is the most common cause of gastroenteritis worldwide. C. jejuni causes campylobacteriosis infection. The typical symptoms include diarrhea with blood in the feces, abdominal pain, fever, headache, nausea and/or vomiting. The infection may be dangerous to young children, the elderly and individuals with immunodeficiency and is most abundant with malnourished children. C. jejuni infections have been associated with severe long-term complications such as Guillain-Barre Syndrome, inflammatory bowel disease and reactive arthritis (see, e.g., Platts-Mills and Kosek, 2014, Curr Opin Infect Dis.; 27(5): 444-450, and references therein).

Typically, C. jejuni infection does not require specific treatment in addition to good supportive care. In more severe cases, in humans and in poultry, the infection has been treated with antibiotics such as fluoroquinoles and macrolides. However, spread of antibiotic-resistant strains is an increasing concern. The treatment with antibiotics is recommended in cases where the bacteria has invaded the intestinal mucosa cell and damaged the tissues, or to eliminate the carrier state. There remains a need for prevention therapies, as well as improved, non-antibiotic, therapies for treatment of the infection. Antibodies targeting C. jejuni have been taught e.g. in International Patent publication WO2014063253, the contents of which are incorporated herein by reference in their entirety.

In some embodiments, methods of the present disclosure may be used to prevent, manage and/or treat infections and complications caused by C. Jejuni.

Viral particles and methods of using the viral particles described in the present disclosure may be used to prevent, manage and/or treat C. Jejuni related infections and/or conditions. As a non-limiting example, the viral particles of the present disclosure comprise a nucleic acid sequence encoding at least one of the sequences described in Table 15.

Clostridium difficile

Clostridium difficile bacteria (C. difficile) is a Gram-positive, anaerobic spore-forming bacteria belonging to the genus of Clostridium. C. difficile inhabits in the soil. C. difficile produces toxins, most commonly enterotoxin A and cytotoxin B. Toxins A and B both have a C-terminal receptor-binding domain containing repeating sequences, a central hydrophobic domain and N-terminal glucosyltranferase domain. The toxins bind to the intestinal epithelial cells leading to glucosylation of target Rho GTPases, disruption of the cytoskeleton and cell death. C. difficile toxins A and B are a common cause C. difficile associated diarrhea and Clostridium difficile colitis, which is an inflammation of the large intestine. Typical symptoms of the colitis include flu-like symptoms, bloating, diarrhea, and/or abdominal pain. The disease may lead to dehydration, kidney failure, bowel perforation, toxic megacolon resulting in colon rupture. The elderly and individuals with a weakened immunity are more susceptible to severe and recurring infections which can be life-threatening. C. difficile is transmitted by the fecal-oral-route. Due to the ability to form heat-resistant spores, the bacteria is not killed by alcohol-based. cleansers or routine surface cleaning. The bacteria may be cultured on almost any surface and survives in clinical environments, such as hospitals. C. difficile is one of the most common and severe healthcare-associated infections. According to CDC, an estimated about half a million infections occur in the United States annually. In 2011, 29,000 deaths related to C. difficile were reported.

Currently C. difficile infections are treated with antibiotics such as vancomycin and metronidazole. How-ever, increasing an antibiotic-resistance to the bacteria is a concern. Especially in cases of recurring infections, antibiotic treatments have an incomplete response and they disrupt the nolinal colonic flora. There remains a need for prevention and improved treatment therapies for the infection. Antibodies targeting C. difficile have been developed. For example, actoxumab and bezlotoxumab (developed by Medarex Inc, and the University of Massachusetts Medical School) are human monoclonal antibodies targeting C. difficile toxin A and toxin B, respectively. The antibodies may be administered as a combination for the prevention of recurring C. difficile infection.

In some embodiments, methods of the present disclosure may be used to prevent, manage and/or treat infections and complications caused by C. difficile.

Viral particles and methods of using the viral particles described in the present disclosure may be used to prevent, manage and/or treat C. difficile related infections and/or conditions. As a non-limiting example, the viral particles of the present disclosure comprise a nucleic acid sequence encoding at least one of the sequences described in Table 14.

Entamoeba histolytica

Entamoeba histolytica (E. histolytica) is an anaerobic one-celled parasite protozoan belonging to the genus of Entamoeba. The active stage of the protozoan exists only in the host and in fresh feces. Cysts survive outside the host in water, soil and food in moist conditions. E. histolytica causes an infection called amebiasis, also known as ameobiasis or entamoebiasis. In majority of cases, amebiasis is symptomless. In 10-20% of individuals infected have symptoms that include loose feces, stomach pain and cramping. The severe more form of amebiasis called amebic dysentery is associated with stomach pain, blood stools and fever. In rare cases, E. histolytica invades the liver, forms an abscess and may spread to other parts of the body, such as the lungs or brain. The transmission to humans is mostly via the fecal-oral-route. The disease is typically caused by ingestion of mature cysts in contaminated food, water or via hands. The disease may also be transmitted in close person-to-person contact, e.g. sexual contact. E. histolytica infections are most common in tropical areas and especially in poor sanitary conditions. It is estimated that 50 million cases of amebiasis occur annually, leading to 100,000 deaths.

As of today, there are no preventive vaccines for E. histolytica infections, though cellular immunity is important for the prevention of liver invasive amebiasis. Amebiasis is typically treated with amebicides, which are medicines targeting E. histolytica at specific parts of the body, e.g. the intestine tissue or liver. Optionally, the treatment may involve one or more antibiotics, as well as steroids. However, increasing antibiotic-resistance of E. histolytica is a concern. There remains a need for prevention therapy as well as for improved treatments. Antibodies targeting E. histolytica are taught in, e.g., 2009, infect limmtm.; 77(1): 549-556, and Tachibana et al., 1999, Clin Diagn Lab Immunol.; 6(3):383-7, the contents of which are incorporated herein by reference in their entirety.

In some embodiments, methods of the present disclosure may be used to prevent, manage and/or treat infections and complications caused by E. histolytica.

Viral particles and methods of using the viral particles described in the present disclosure may be used to prevent, manage and/or treat E. histolytica related infections and/or conditions. As a non-limiting example, the viral particles of the present disclosure comprise a nucleic acid sequence encoding at least one of the sequences described in Table 20.

Helicobacter pyroli

Helicobacter pyroli (H. pyroli) is a Gram-negative, spiral-shaped microaerophilic bacterium. H. pyroli infection is typically asymptomatic and is suggested to be transmitted through the fecal-oral route or oral-oral route. According to CDC, two-thirds of the world's population is infected with H. pyroli. The infection may cause chronic active, chronic, persistent, and atrophic gastritis, duodenal and gastric ulcers and is associated with cancer. CDC reposts 25 million Americans suffering from an ulcer during their lifetime. Typical symptoms associated with ulcer are gnawing or burning pain in the epigastrium, especially between meals. Additional symptoms include nausea, vomiting, loss of appetite, internal bleeding leading to anemia and fatigue.

Typical treatment for d. pyroli infection involves antibiotics. Increasing antibiotic resistance and patient noncompliance are major challenges associated with the antibiotic treatment. There remains a need for improved, non-antibiotic, treatment and prevention therapies targeting H. Pyroli. Antibodies targeting H. pyroli infection have been developed. For example, Boren et al. teach antibodies targeting the BAbA antigen expressed by H. pyroli in US patent US8025880, the contents of which are incorporated herein by reference in their entirety.

In some embodiments, methods of the present disclosure may be used to prevent, manage and/or treat infections and complications caused by H. pyroli.

Viral particles and methods of using the viral particles described in the present disclosure may be used to prevent, manage and/or treat H. pyroli related infections and/or conditions. As a non-limiting example, the viral particles of the present disclosure comprise a nucleic acid sequence encoding at least one of the sequences described in Table 16.

### Enterotoxin B

Enterotoxin B is a toxin produced by certain strains of Gram-positive bacteria Staphylococcus (wrens and is a common cause for food poisoning. Staphylococcus species thrive and produce toxins in unrefrigerated meats, dairy, and bakery products. The symptoms associated with enterotoxin B infection are severe diarrhea, nausea and intestinal cramping. The toxin may remain active in the human body after the bacteria has been killed. Enterotoxin B is a so-called superantigen. Superantigens are toxins that may activate T cells by forming a bridge between a MEC II on antigen presenting cells (APCs) and the T cell receptors (TCR). Due to binding of enterotoxin B, the T cells release large amount of cytokines leading to an inflammation and gastroenteritis. Though enterotoxin B infection is typically not life threatening, enterotoxin B has been identified as a potential chemical and biological warfare agent.

As of today, there is no specific prevention or treatment for enterotoxin B infection. Antibodies that neutralize enterotoxin B have been investigated, e.g. as described in U.S. Pat. No. 8,895,704.

In some embodiments, methods of the present disclosure may be used to prevent, manage and/or treat infections and complications caused by enterotoxin B.

Viral particles and methods of using the viral particles described in the present disclosure may be used to prevent, manage and/or treat enterotoxin B related infections and/or conditions. As a non-limiting example, the viral particles of the present disclosure comprise a nucleic acid sequence encoding at least one of the sequences described in Table 16.

### V. Kits and Devices

### Kits

In some embodiments, the disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

Any of the viral particles of the present disclosure may be comprised in a kit. In some embodiments, kits may further include reagents and/or instructions for creating and/or synthesizing compounds and/or compositions of the present disclosure. In some embodiments, kits may also include one or more buffers. In some embodiments, kits of the disclosure may include components for making protein or nucleic acid arrays or libraries and thus, may include, for example, solid supports.

In some embodiments, kit components may be packaged either in aqueous media or in lyophilized form. The container means of the kits will generally include at least one vial, test tube, flask, bottle, syringe or other container means, into which a component may be placed, and preferably, suitably aliquoted. Where there is more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present disclosure may also typically include means for containing compounds and/or compositions of the present disclosure, e.g., proteins, nucleic acids, and any other reagent containers in close confinement for commercial sale. Such containers may include injection or blow-molded plastic containers into which desired vials are retained.

In some embodiments, kit components are provided in one and/or more liquid solutions. In some embodiments, liquid solutions are aqueous solutions, with sterile aqueous solutions being particularly preferred. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the disclosure. In such embodiments, dye may then be resuspended in any suitable solvent, such as DMSO.

In some embodiments, kits may include instructions for employing kit components as well the use of any other reagent not included in the kit. Instructions may include variations that may be implemented.

### Devices

In some embodiments, the viral particles may be delivered to a subject using a device to deliver the viral particles and a head fixation assembly. The head fixation assembly may be, but is not limited to, any of the head fixation assemblies sold by MRI interventions. As a non-limiting example, the head fixation assembly may be any of the assemblies described in U.S. Pat. Nos. 8,099,150, 8,548,569, and 9,031,636 and International Patent Publication Nos. WO201108495 and WO2014014585, the contents of each of which are incorporated by reference in their entireties. A head fixation assembly may be used in combination with an MM compatible drill such as, but not limited to, the MRI, compatible drills described in International Patent Publication No. WO2013181008 and US Patent Publication No. US20130325012, the contents of which are herein incorporated by reference in its entirety.

In some embodiments, the viral particles may be delivered using a method, system and/or computer program for positioning apparatus to a target point on a subject to deliver the viral particles. As a non-limiting example, the method, system and/or computer program may be the methods, systems and/or computer programs described in U.S. Pat. No. 8,340,743, the contents of which are herein incorporated by reference in its entirety. The method may include: determining a target point in the body and a reference point, wherein the target point and the reference point define a planned trajectory line (PTL) extending through each; determining a visualization plane, wherein the PTL intersects the visualization plane at a sighting point; mounting the guide device relative to the body to move with respect to the PTL, wherein the guide device does not intersect the visualization plane; determining a point of intersection (GPP) between the guide axis and the visualization plane; and aligning the GPP with the sighting point in the visualization plane.

In some embodiments, the viral particles may be delivered to a subject using a convention-enhanced delivery device. Non-limiting examples of targeted delivery of drugs using convection are described in US Patent Publication Nos. US20100217228, US20130035574, and US 20130035660 and international Patent Publication No. WO2013019830 and WO2008144585, the contents of each of which are herein incorporated by reference in their entireties.

In some embodiments, a subject may be imaged prior to, during and/or after delivery of the viral particles. The imaging method may be a method known in the art and/or described herein, such as but not limited to, magnetic resonance imaging (MRI) As a non-limiting example, imaging may be used to assess therapeutic effect. As another non-limiting example, imaging may be used for assisted delivery of viral particles.

In some embodiments, the viral particles may be delivered using an MRI-guided device. Non-limiting examples of MRI-guided devices are described in U.S. Pat. Nos. 9,055,884, 9,042,958, 8,886,288, 8,768,433, 8,396,532, 8,369,930, 8,374,677, and 8,175,677 and US Patent Application No. US20140024927 the contents of each of which are herein incorporated by reference in their entireties. As a non-limiting example, the MRI-guided device may be able to provide data in real time such as those described in U.S. Pat. Nos. 8,886,288 and 8,768,433, the contents of each of which is herein incorporated by reference in its entirety. As another non-limiting example, the MM-guided device or system may be used with a targeting cannula such as the systems described in U.S. Pat. Nos. 8,175,677 and 8,374,677, the contents of each of which are herein incorporated by reference in their entireties. As yet another non-limiting example, the MRI-guided device includes a trajectory guide frame for guiding an interventional device as described, for example, in U.S. Pat. No. 9,055,884 and US Patent Application No. US20140024927, the contents of each of which are herein incorporated by reference in their entireties.

In some embodiments, the viral particles may be delivered using an MM-compatible tip assembly. Non-limiting examples of MRI-compatible tip assemblies are described in US Patent Publication No. US20140275980, the contents of which is herein incorporated by reference in its entirety.

In some embodiments, the viral particles may be delivered using a cannula which is MRI-compatible. Non-limiting examples of MRI-compatible cannulas include those taught in International Patent Publication No. WO2011130107, the contents of which are herein incorporated by reference in its entirety.

In some embodiments, the viral particles may be delivered using a catheter which is MRI-compatible. Non-limiting examples of MM-compatible catheters include those taught in International Patent Publication No. WO2012116265, U.S. Pat. No. 8,825,133 and US Patent Publication No. US20140024909, the contents of each of which are herein incorporated by reference in their entireties.

In some embodiments, the viral particles may be delivered using a device with an elongated tubular body and a diaphragm as described in US Patent Publication Nos. US20140276582 and US20140276614, the contents of each of which are herein incorporated by reference in their entireties.

In some embodiments, the viral particles may be delivered using an MRI compatible localization and/or guidance system such as, but not limited to, those described in US Patent Publication Nos. US20150223905 and US20150230871, the contents of each of which are herein incorporated by reference in their entireties. As a non-limiting example, the MRI compatible localization and/or guidance systems may comprise a mount adapted for fixation to a patient, a targeting cannula with a lumen configured to attach to the mount so as to be able to controllably translate in at least three dimensions, and an elongate probe configured to snugly advance via slide and retract in the targeting cannula lumen, the elongate probe comprising at least one of a stimulation or recording electrode.

In some embodiments, the viral particles may be delivered to a subject using a trajectory frame as described in US Patent Publication Nos. US20150031982 and US20140066750 and International Patent Publication Nos. WO2015057807 and WO2014039481, the contents of each of which are herein incorporated by reference in their entireties.

In some embodiments, the viral particles may be delivered to a subject using a gene gun.

### VI. Definitions

At various places in the present specification, substituents of compounds of the present disclosure are disclosed in groups or in ranges. It is specifically intended that the present disclosure include each and every individual subcombination of the members of such groups and ranges.

About: As used herein, the term “about” means+/−10% of the recited value.

Adeno-associated virus: The term “adeno-associated virus” or “AAV” as used herein refers to members of the dependovirus genus comprising any particle, sequence, gene, protein, or component derived therefrom.

AAV Particle: As used herein, an “AAV particle” is a virus which comprises a viral genome with at least one payload region and at least one ITR region. AAV vectors of the present disclosure may be produced recombinantly and may be based on adeno-associated virus (AAV) parent or reference sequences. AAV particle may be derived from any serotype, described herein or known in the art, including combinations of serotypes (i.e., “pseudotyped” AAV) or from various genomes (e.g., single stranded or self-complementary). In addition, the AAV particle may be replication defective and/or targeted.

Activity: As used herein, the term “activity” refers to the condition in which things are happening or being done. Compositions of the disclosure may have activity and this activity may involve one or more biological events.

Administered in combination: As used herein, the term “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Animal: As used herein, the term “animal” refers to any member of the animal kingdom. In some embodiments, “animal” refers to humans at any stage of development. In some embodiments, “animal” refers to nonhuman animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Antibody: As used herein, the term “antibody” is referred to in the broadest sense and specifically covers various embodiments including, but not limited to monoclonal antibodies, polyclonal antibodies, multispecific antibodies (e.g. bispecific antibodies formed from at least two intact antibodies), and antibody fragments (e.g., diabodies) so long as they exhibit a desired biological activity (e.g., “functional”). Antibodies are primarily amino-acid based molecules but may also comprise one or more modifications (including, but not limited to the addition of sugar moieties, fluorescent moieties, chemical tags, etc.). Non-limiting examples of antibodies or fragments thereof include V_{H} and V_{L} domains, scFvs, Fab, Fab′, F(ab′)₂, Fv fragment, diabodies, linear antibodies, single chain antibody molecules, multispecific antibodies, bispecifrc antibodies, intrabodies, monoclonal antibodies, polyclonal antibodies, humanized antibodies, codon-optimized antibodies, tandem say antibodies, bispecific T-cell engagers, mAb2 antibodies, chimeric antigen receptors (CAR), tetravalent bispecific antibodies, biosynthetic antibodies, native antibodies, miniaturized antibodies, unibodies, maxibodies, antibodies to senescent cells, antibodies to conformers, antibodies to disease specific epitopes, or antibodies to innate defense molecules.

Antibody-based composition: As used herein, “antibody-based” or “antibody-derived” compositions are monomeric or multimeric polypeptides which comprise at least one amino-acid region derived from a known or parental antibody sequence and at least one amino acid region derived from a non-antibody sequence, e.g., mammalian protein.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the wills “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Bifunctional: As used herein, the term “bifunctional” refers to any substance, molecule or moiety which is capable of or maintains at least two functions. The functions may affect the same outcome or a different outcome. The structure that produces the function may be the same or different.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, a viral particle of the present disclosure may be considered biologically active if even a portion of the encoded payload is biologically active or mimics an activity considered biologically relevant.

Capsid: As used herein, the term “capsid” refers to the protein shell of a virus particle.

Chimeric antigen receptor (CAR): As used herein, the term “chimeric antigen receptor” or “CAR” refers to an artificial chimeric protein comprising at least one antigen specific targeting region (ASTR), a transmembrane domain and an intracellular signaling domain, wherein the antigen specific targeting region comprises a full-length antibody or a fragment thereof. As a non-limiting example the ASTR of a CAR may be any of the antibodies listed in Tables 3-53, antibody-based compositions or fragments thereof. Any molecule that is capable of binding a target antigen with high affinity can be used in the ASTR of a CAR. The CAR may optionally have an extracellular spacer domain and/or a co-stimulatory domain. A CAR may also be used to generate a cytotoxic cell carrying the CAR.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, and vice versa, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mess, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

Compound: Compounds of the present disclosure include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Comprehensive Positional Evolution (CPE™): As used herein, the term “comprehensive positional evolution” refers to an antibody evolution technology that allows for mapping of the effects of amino acid changes at every position along an antibody variable domain's sequence. This comprehensive mutagenesis technology can be used to enhance one or more antibody properties or characteristics.

Comprehensive Protein Synthesis (CPS™): As used herein, the term “comprehensive protein synthesis” refers to a combinatorial protein synthesis technology that can be used to optimize antibody properties or characteristics by combining the best properties into a new, high-performance antibody.

Conditionally active: As used herein, the term “conditionally active” refers to a mutant or variant of a wild-type polypeptide, wherein the mutant or variant is more or less active at physiological conditions than the parent polypeptide. Further, the conditionally active polypeptide may have increased or decreased activity at aberrant conditions as compared to the parent polypeptide. A conditionally active polypeptide may be reversibly or irreversibly inactivated at normal physiological conditions or aberrant conditions.

Conserved. As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70?/(identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of a polynucleotide or polypeptide or may apply to a portion, region or feature thereof.

Control Elements: As used herein, “control elements”, “regulatory control elements”, or “regulatory sequences” refers to promoter regions, polyadenylation signals, transcription termination sequences, upstream regulatory domains, origins of replication, internal ribosome entry sites (“TRES”), enhancers, and the like, which provide for the replication, transcription and translation of a coding sequence in a recipient cell. Not all of these control elements need always be present as long as the selected coding sequence is capable of being replicated, transcribed and/or translated in an appropriate host cell.

Controlled Release: As used herein, the term “controlled release” refers to a pharmaceutical composition or compound release profile that conforms to a particular pattern of release to effect a therapeutic outcome.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering a viral particle, a compound, substance, entity, moiety, cargo or payload.

Delivery Agent: As used herein, “delivery agent” refers to any substance which facilitates, at least in part, the in vivo delivery of a viral particle to targeted cells.

Destabilized: As used herein, the term “destable”, “destabilize”, or “destabilizing region” means a region or molecule that is less stable than a starting, wild-type or native form of the same region or molecule.

Delectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the peptides or proteins disclosed herein. They may be within the amino acids, the peptides, or proteins, or located at the N- or C-termini.

Digest: As used herein, the term “digest” means to break apart into smaller pieces or components. When referring to polypeptides or proteins, digestion results in the production of peptides.

Distal: As used herein, the term “distal” means situated away from the center or away from a point or region of interest.

Dosing regimen: As used herein, a “dosing regimen” is a schedule of administration or physician determined regimen of treatment, prophylaxis, or palliative care.

Encapsulate: As used herein, the term “encapsulate” means to enclose, surround or encase.

Engineered: As used herein, embodiments of the disclosure are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Epitope: As used herein, an “epitope” refers to a surface or region on a molecule that is capable of interacting with a biomolecule. For example, a protein may contain one or more amino acids, e.g., an epitope, which interacts with an antibody, e.g., a biomolecule. In some embodiments, when referring to a protein or protein module, an epitope may comprise a linear stretch of amino acids or a three-dimensional structure formed by folded amino acid chains.

EvoMap™: As used herein, an EvoMap™ refers to a map of a polypeptide, wherein detailed informatics are presented about the effects of single amino acid mutations within the length of the polypeptide and their influence on the properties and characteristics of that polypeptide.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least one viral particle and a. delivery agent.

Fragment: A “fragment,” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RINA, mRNA, polypeptides and peptides are well known in the art.

Homology: As used herein, the term “homology” refers to the overall relatedness between polymeric molecules, e.g. between polynucleotide molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids.

Heterologous Region: As used herein the term “heterologous region” refers to a region which would not be considered a homologous region.

Homologous Region: As used herein the term “homologous region” refers to a region which is similar in position, structure, evolution origin, character, form or function.

Identity As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G, eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the (SCG software package using an NWSgapdna.CMP matrix. Methods commonly employed to determine percent identity between sequences include, but are not limited to those disclosed in Carillo, H. and Lipman, D., SIAM J Applied Math., 48:1073 (1988); incorporated herein by reference. Techniques for determining identity are codified in publicly available computer programs, Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting), Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that a substance is substantially separated from the environment in which it was formed or detected. Partial separation can include, for example, a composition enriched in the substance or viral particles of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Linker: As used herein “linker” refers to a molecule or group of molecules which connects two molecules, such as a V_{H} chain and V_{L} chain or an antibody. A linker may be a nucleic acid sequence connecting two nucleic acid sequences encoding two different polypeptides. The linker may or may not be translated. The linker may be a cleavable linker.

MicroRNA (miRNA) binding site: As used herein, a microRNA (miRNA) binding site represents a nucleotide location or region of a nucleic acid transcript to which at least the “seed” region of a miRNA binds.

Modified. As used herein “modified” refers to a changed state or structure of a molecule of the disclosure. Molecules may be modified in many ways including chemically, structurally, and functionally.

Naturally Occurring: As used herein, “naturally occurring” or “wild-type” means existing in nature without artificial aid, or involvement of the hand of man.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Off-target: As used herein, “off target” refers to any unintended effect on any one or more target, gene, or cellular transcript.

Open reading frame: As used herein, “open reading frame” or “ORF” refers to a sequence which does not contain a stop codon in a given reading frame.

Operably linked. As used herein, the phrase “operably linked” refers to a functional connection between two or more molecules, constructs, transcripts, entities, moieties or the like.

Particle: As used herein, a “particle” is a virus comprised of at least two components, a protein capsid and a polynucleotide sequence enclosed within the capsid.

Patient: As used herein, “patient” refers to a subject who may seek or be in need of treatment, requires treatment, is receiving treatment, will receive treatment, or a subject who is under care by a trained professional for a particular disease or condition.

Payload: As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid.

Payload construct: As used herein, “payload construct” is one or more polynucleotide regions encoding or comprising a payload that is flanked on one or both sides by an inverted terminal repeat (ITR) sequence. The payload construct is a template that is replicated in a viral production cell to produce a viral genome.

Payload construct vector: As used herein, “payload construct vector” is a vector encoding or comprising a payload construct, and regulatory regions for replication and expression in bacterial cells.

Payload construct expression vector: As used herein, a “payload construct expression vector” is a vector encoding or comprising a payload construct and which further comprises one or more polynucleotide regions encoding or comprising components for viral expression in a viral replication cell.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, di gluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethyl amine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the disclosure wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono, di-, and tri-hydrates), AI-methylpyrrolidinone (NMP), dimethyl sulfoxide (DMSO), N,N′-dimethylformamide (DMF), N,N″-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Protein of interest: As used herein, the terms “proteins of interest” or “desired proteins” include those provided herein and fragments, mutants, variants, and alterations thereof.

Proximal: As used herein, the term “proximal” means situated nearer to the center or to a point or region of interest.

Purified. As used herein, “purify,” “purified,” “purification” means to make substantially pure or clear from unwanted components, material defilement, admixture or imperfection. “Purified” refers to the state of being pure. “Purification” refers to the process of making pure.

Region: As used herein, the term “region” refers to a zone or general area. In some embodiments, when referring to a protein or protein module, a region may comprise a linear sequence of amino acids along the protein or protein module or may comprise a three-dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may there for comprise the N- and/or C-termini as well as surrounding amino acids. In some embodiments, N- and/or C-terminal regions comprise from about 3 amino acid to about 30 amino acids, from about 5 amino acids to about 40 amino acids, from about 10 amino acids to about 50 amino acids, from about 20 amino acids to about 100 amino acids and/or at least 100 amino acids. In some embodiments, N-terminal regions may comprise any length of amino acids that includes the N-terminus, but does not include the C-terminus. In some embodiments, C-terminal regions may comprise any length of amino acids, which include the C-terminus, but do not comprise the N-terminus.

In some embodiments, when referring to a polynucleotide, a region may comprise a linear sequence of nucleic acids along the polynucleotide or may comprise a three-dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for comprise the 5′ and 3′ termini as well as surrounding nucleic acids. In some embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In some embodiments, 5′ regions may comprise any length of nucleic acids that includes the 5′ terminus, but does not include the 3′ terminus. In some embodiments, 3′ regions may comprise any length of nucleic acids, which include the 3′ terminus, but does not comprise the 5′ terminus.

RNA or RATA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DINA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Self-complementary viral particle: As used herein, a “self-complementary viral particle” is a particle comprised of at least two components, a protein capsid and a polynucleotide sequence encoding a self-complementary genome enclosed within the capsid.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event. In some embodiments, a single unit dose is provided as a discrete dosage fo (e.g., a tablet, capsule, patch, loaded syringe, vial, etc.).

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules of the present disclosure may be chemical or enzymatic.

Targeting: As used herein, “targeting” means the process of design and selection of nucleic acid sequence that will hybridize to a target nucleic acid and induce a desired effect.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Therapeutically of outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hr period. It may be administered as a single unit dose.

Transfection: As used herein, the term “transfection” refers to methods to introduce exogenous nucleic acids into a cell. Methods of transfection include, but are not limited to, chemical methods, physical treatments and cationic lipids or mixtures.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Vector: As used herein, a “vector” is any molecule or moiety which transports, transduces or otherwise acts as a carrier of a heterologous molecule. Vectors of the present disclosure may be produced recombinantly and may be based on and/or may comprise adeno-associated virus AAV) parent or reference sequence. Such parent or reference AAV sequences may serve as an original, second, third or subsequent sequence for engineering vectors. In non-limiting examples, such parent or reference AAV sequences may comprise any one or more of the following sequences: a polynucleotide sequence encoding a polypeptide or multi-polypeptide, which sequence may be wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence. These AAV sequences may serve as either the “donor” sequence of one or more codons (at the nucleic acid level) or amino acids (at the polypeptide level) or “acceptor” sequences of one or more codons (at the nucleic acid level) or amino acids (at the polypeptide level).

Viral genome: As used herein, a “viral genome” or “vector genome” is a polynucleotide comprising at least one inverted terminal repeat (ITR) and at least one encoded payload. A viral genome encodes at least one copy of the payload.

Described herein are compositions, methods, processes, kits and devices for the design, preparation, manufacture and/or formulation of viral particles. In some embodiments, payloads, such as but not limited to polynucleotides, may be encoded by payload constructs or contained within plasmids or vectors or recombinant viruses (e.g., AAVs, lentivirus, or retrovirus).

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

The present disclosure is further illustrated by the following non-limiting examples.

### VII. Examples

### Example 1. Production and Purification of AAV Particles

AAV particles described herein may be produced using methods known in the art, such as, for example, triple transfection or baculovirus mediated virus production. Any suitable permissive or packaging cell known in the art may be employed to produce the vectors. Mammalian cells are often preferred. Also preferred are trans-complementing packaging cell lines that provide functions deleted from a replication-defective helper virus, e.g., 293 cells or other E1a trans-complementing cells.

The gene cassette may contain some or all of the parvovirus (e.g., AAV) cap and rep genes. Preferably, however, some or all of the cap and rep functions are provided in trans by introducing a packaging vector(s) encoding the capsid and/or Rep proteins into the cell, Most preferably, the gene cassette does not encode the capsid or Rep proteins. Alternatively, a packaging cell line is used that is stably transformed to express the cap and/or rep genes

Recombinant AAV virus particles are, in some cases, produced and purified from culture supernatants according to the procedure as described in US20160032254, the contents of which are incorporated by reference, Production may also involve methods known in the art including those using 293T cell, sf9 insect cells, triple transfection or any suitable production method.

In some cases, 293 cells are transfected with CaPO4 with plasmids required for production of AAV, i.e., AAV2 rep, an adenoviral helper construct and a ITR flanked transgene cassette. The AAV2 rep plasmid also contains the cap sequence of the particular virus being studied. Twenty-four hours after transfection, which occurs in serum containing DMEM, the medium is replaced with fresh medium with or without serum. Three (3) days after transfection, a sample is taken from the culture medium of the 293 adherent cells. Subsequently cells are scraped and transferred into a receptacle. After centrifugation to remove cellular pellet, a second sample is taken from the supernatant after scraping. Next cell lysis is achieved by three consecutive freeze-thaw cycles (−80C. to 37C.). Cellular debris is removed and sample 3 is taken from the medium. The samples are quantified for AAV particles by DNase resistant genome titration by Taqman™. PCR. The total production yield from such a transfection is equal to the particle concentration from sample 3.

AAV vector titers are measured according to genome copy number (genome particles per milliliter). Genome particle concentrations are based on Taqman® PCR of the vector DNA as previously reported (Clark et al. (1999) Hum. Gene Ther., 10:1031-1039; Veldwijk et al. (2002) Mol. Ther., 6:272-278).

### Example 2. Tissue Specific Expression

To evaluate the expression of various encoded antibody payloads in tissues, a series of AAV particles carrying the encoded antibody sequences driven by a panel of ubiquitous and tissue-specific promoters are made. These particles are administered to the specific tissue, e.g., intramuscularly, via an appropriate route, e.g., a single injection in the gastrocnemius muscle and expression is monitored to determine the relative expression potential of the payload as well as of each promoter in this target tissue. Measurement of antibody production is performed using standard techniques, for example by ELISA.

In some cases, the cytomegalovirus immediate early promoter (CMV), chimeric chicken-beta-actin (CAG), and ubiquitin C (UBC), CBA, Hi promoters provide robust expression.

### Example 3. Generation of Antibodies

### Antibody Production by Hybridoma Technology

Host animals (e.g. mice, rabbits, goats, and llamas) are immunized by an injection with an antigenic protein (e.g., tau) to elicit lymphocytes that specifically bind to the antigen (e.g., tau). Lymphocytes are collected and fused with immortalized cell lines to generate hybridomas. Hybridomas are cultured in a suitable culture medium that is enriched with appropriate selection agents to promote growth.

Antibodies produced by the cultured hybridomas are subjected to analysis to determine binding specificity of the antibodies for the target antigen. Once antibodies with desirable characteristics are identified, corresponding hybridomas are subcloned through limiting dilution procedures and grown by standard methods. Antibodies produced by these cells are isolated and purified using standard immunoglobulin purification procedures.

### Recombinant Antibody Production

Recombinant antibodies are produced using heavy and light chain variable region cDNA sequences selected from hybridomas or from other sources. Sequences encoding antibody variable domains expressed by hybridomas are determined by extracting RNA molecules from antibody-producing hybridoma cells and producing cDNA by reverse transcriptase polymerase chain reaction (PCR). PCR is used to amplify cDNA using primers specific for heavy and light chain sequences. PCR products are then subcloned into plasmids for sequence analysis. Antibodies are produced by insertion of resulting variable domain sequences into expression vectors.

Recombinant antibodies are also produced using phage display technology. Target antigens are screened, in vitro, using phage display libraries having millions to billions of phage particles expressing unique single chain variable fragments (scFvs) on their viral coat. Precipitated phage particles are analyzed and sequences encoding expressed says are determined. Sequences encoding antibody variable domains and/or CDRs are inserted into expression vectors for antibody production.

Recombinant antibodies are further produced using yeast surface display technology, wherein antibody variable domain sequences are expressed on the cell surface of Saccharomyces cerevisiae. Recombinant antibodies are developed by displaying the antibody fragment of interest as a fusion to e.g. Aga2p protein on the surface of the yeast, where the protein interacts with proteins and small molecules in a solution. says with affinity towards desired receptors are isolated from the yeast surface using magnetic separation and flow cytometry. Several cycles of yeast surface display and isolation will be done to attain scFvs with desired properties through directed evolution.

### Example 4. Optimization of the Encoded Antibody

To design an optimal framework for the expression of an antibody, the heavy and light chains of several antibodies separated by an F2A self-processing peptide sequence are cloned into a mammalian expression vector under the control of the CMV promoter. 293T cells or any suitable cell line transfected with these vectors exhibit secretion of human IgG into the culture supernatant that is then detected by ELISA.

To increase expression, the antibody chains and/or the processing peptide are codon optimized for mammalian expression. In some instances, a furin cleavage site at the N-terminus is inserted for better processing.

To improve secretion of the antibody, the endogenous signal sequences are replaced with a sequence which may or may not be codon optimized, derived from any gene. In some cases, the human growth hormone signal sequence is used. Any of the heavy, light or both chains may be driven by any signal sequence, whether the same or different. Antibody expression is confirmed using standard immunohistochemical techniques, including ELISA.

### Example 5. Vectored Antibodies

Viral genomes are designed for AAV delivery of antibodies to cells. The viral genome comprises a payload region and at least one inverted terminal repeat (ITR) region. The payload region may optionally encode regulatory elements e.g., a promoter region, an intronic region, or a polyadenylation sequence. The payload region comprises a sequence encoding one or more polypeptides selected from the group consisting of those listed in Tables 3-53. An exemplary payload region comprises a sequence encoding an antibody heavy chain, a region encoding an antibody light chain and a region encoding a linker connecting the heavy and light chain sequences or polypeptides before further processing. A promoter is selected to target the desired tissue or for desired regulation of expression, or both. The promoter may be selected from human EF1a, CMV, CBA, and its derivative CAG, GUSB, UBC, or any other promoter known to one with skill in the art, or combinations thereof. The 5′ and 3′ ITRs may or may not be of the same serotype as the capsid of the AAV particle.

Payload regions may optionally encode a linker between light and heavy antibody chain sequences or polypeptides. Sequence encoding linkers are derived from an internal ribosome entry site (IRES), foot and mouth disease virus 2A (F2A), porcine teschovirus-1 virus 2A (P2A), a furin cleavage site (F), or a 5xG4S linker sequence (SEQ ID N0: 32689 or SEQ ID NO: 1728). In various payload regions, the order of heavy and light chains is alternated with respect to 5′ to 3′ direction. Payloads are further designed to encode protein signal sequences (to aid in protein processing, localization, and/or secretion) as well as an untranslated poly A tail.

Each viral genome is then incorporated into an AAV cloning vector to create payload expression vectors.

The payload expression vectors are expressed in e.g. Expi293 cells. The supernatants are collected and expressed antibodies are purified using protein A/G beads. Supernatants are diluted with a loading buffer and applied to a column prepared with A/G beads. Unbound proteins are washed through with loading buffer. Elution buffer is added to the column, fractions collected, and fractions containing proteins of interest are identified with absorption spectroscopy technique, pooled together, and neutralized. Western blotting techniques are used to identify payload regions producing the antibody proteins of interest. Purified antibodies are then tested for their affinity to their specific target by e.g. ELISA essay technique and antibodies with the highest affinity are identified and selected.

Finally, the rAAVs are produced using, for example, HEK293T cells. The cells are transfected simultaneously with the viral genome of the present disclosure, a viral genome encoding helper proteins and a viral genome encoding replication and capsid proteins.

### Example 6. In Vivo Expression and Efficacy of Antibody Payloads

To determine the efficacy or comparative expression of encoded antibodies, dose-dependent expression is determined at a series of time points. Samples from mice treated with AAV particles encoding antibodies or luciferase at various levels are examined for expression using standard techniques such as nucleic acid analyses for RNA levels, protein analyses for antibody levels and compared to the expression of the luciferase control.

### Example 7. Generation of VA-DER Systems

The vectored augmentation systems and or methods of the present disclosure include at least a TRIM21 protein or a nucleic acid sequence encoding a TRIM21 protein or fragment or variant thereof.

TRIM21 sequences include those in Table 58.

TABLE 58
TRIM21 Sequences

| Sequence | SEQ ID | |
|---|---|---|
| Type | NO | SEQUENCE |
| mRNA | 32670 | >NM_003141.3 Homo sapiens |
| | | tripartite motif containing |
| | | 21 (TRIM21), mRNA |
| | | GCTTCTGAGCGGAAACTGAAAGTGAAATAG |
| | | GGAGCTGGCTACCAGCGTTGAGTCCCCTGT |
| | | AAAGCCAAACCCCCTAAAGGTCTCCACACT |
| | | GCTGTTTAACGGCACACTTGACAATGGCTT |
| | | CAGCAGCACGCTTGACAATGATGTGGGAGG |
| | | AGGTCACATGCCCTATCTGCCTGGACCCCT |
| | | TCGTGGAGCCTGTGAGCATCGAGTGTGGCC |
| | | ACAGCTTCTGCCAGGAATGCATCTCTCAGG |
| | | TTGGGAAAGGTGGGGGCAGCGTCTGTCCTG |
| | | TGTGCCGGCAGCGCTTTCTGCTCAAGAATC |
| | | TCCGGCCCAATCGACAGCTAGCCAACATGG |
| | | TGAACAACCTTAAAGAAATCAGCCAGGAGG |
| | | CCAGAGAGGGCACACAGGGGGAACGGTGTG |
| | | CAGTGCATGGAGAGAGACTTCACCTGTTCT |
| | | GTGAGAAAGATGGGAAGGCCCTTTGCTGGG |
| | | TATGTGCCCAGTCTCGGAAACACCGTGACC |
| | | ACGCCATGGTCCCTCTTGAGGAGGCTGCAC |
| | | AGGAGTACCAGGAGAAGCTCCAGGTGGCAT |
| | | TAGGGGAACTGAGAAGAAAGCAGGAGTTGG |
| | | CTGAGAAGTTGGAAGTGGAAATTGCAATAA |
| | | AGAGAGCAGACTGGAAGAAAACAGTGGAAA |
| | | CACAGAAATCTAGGATTCACGCAGAGTTTG |
| | | TGCAGCAAAAAAACTTCCTGGTTGAAGAAG |
| | | AACAGAGGCAGCTGCAGGAGCTGGAGAAGG |
| | | ATGAGAGGGAGCAGCTGAGAATCCTGGGGG |
| | | AGAAAGAGGCCAAGCTGGCCCAGCAGAGCC |
| | | AGGCCCTACAGGAGCTCATCTCAGAGCTAG |
| | | ATCGAAGGTGCCACAGCTCAGCACTGGAAC |
| | | TGCTGCAGGAGGTGATAATTGTCCTGGAAA |
| | | GGAGTGAGTCCTGGAACCTGAAGGACCTGG |
| | | ATATTACCTCTCCAGAACTCAGGAGTGTGT |
| | | GCCATGTGCCAGGGCTGAAGAAGATGCTGA |
| | | GGACATGTGCAGTCCACATCACTCTGGATC |
| | | CAGACACAGCCAATCCGTGGCTGATACTTT |
| | | CAGAAGATCGGAGACAAGTGAGGCTTGGAG |
| | | ACACCCAGCAGAGCATACCTGGAAATGAAG |
| | | AGAGATTTGATAGTTATCCTATGGTCCTGG |
| | | GTGCCCAGCACTTTCACTCTGGAAAACATT |
| | | ACTGGGAGGTAGATGTGACAGGAAAGGAGG |
| | | CCTGGGACCTGGGTGTCTGCAGAGACTCTG |
| | | TGCGCAGGAAGGGGCACTTTTTGCTTAGTT |
| | | CCAAGAGTGGCTTCTGGACAATTTGGTTGT |
| | | GGAACAAACAAAAATATGAGGCTGGCACCT |
| | | ACCCCCAGACTCCCCTCCACCTTCAGGTGC |
| | | CTCCATGCCAAGTTGGGATTTTCCTGGACT |
| | | ATGAGGCTGGCATGGTCTCCTTCTACAACA |
| | | TCACTGACCATGGCTCCCTCATCTACTCCT |
| | | TCTCTGAATGTGCCTTTACAGGACCTCTGC |
| | | GGCCCTTCTTCAGTCCTGGTTTCAATGATG |
| | | GAGGAAAAAACACAGCCCCTCTAACCCTCT |
| | | GTCCACTGAATATTGGATCACAAGGATCCA |
| | | CTGACTATTGATGGCTTTCTCTGGACACTG |
| | | CCACTCTCCCCATTGGCACCGCTTCTCAGC |
| | | CACAAACCCTGCCTCTTTTCCCCATGAACT |
| | | CTGAACCACCTTTGTCTCTGCAGAGGCATC |
| | | CGGATCCCAGCAAGCGAGCTTTAGCAGGGA |
| | | AGTCACTTCACCATCAACATTCCTGCCCCA |
| | | GATGGCTTTGTGATTCCCTCCAGTGAAGCA |
| | | GCCTCCTTATATTTGGCCCAAACTCATCTT |
| | | GATCAACCAAAAACATGTTTCTGCCTTCTT |
| | | TATGGGACTTAAGTTTTTTTTTTCTCCTCT |
| | | CCATCTCTAGGATGTCGTCTTTGGTGAGAT |
| | | CTCTATTATATCTTGTATGGTTTGCAAAAG |
| | | GGCTTCCTAAAAATAAAAAATAAAATTTAA |
| | | AAAACTGTGAAAAAAAAAAAAAAAAA |
| Protein | 32671 | >NP_003132.2 E3 ubiquitin- |
| | | protein ligase TRIM21 |
| | | [Homo sapiens] |
| | | MASAARLTMMWEEVTCPICLDPFVEPVSIE |
| | | CGHSFCQECISQVGKGGGSVCPVCRQRFLL |
| | | KNLRPNRQLANMVNNLKEISQEAREGTQGE |
| | | RCAVHGERLHLFCEKDGKALCWVCAQSRKH |
| | | RDHAMVPLEEAAQEYQEKLQVALGELRRKQ |
| | | ELAEKLEVEIAIKRADWKKTVETQKSRIHA |
| | | EFVQQKNFLVEEEQRQLQELEKDEREQLRI |
| | | LGEKEAKLAQQSQALQELISELDRRCHSSA |
| | | LELLQEVIIVLERSESWNLKDLDITSPELR |
| | | SVCHVPGLKKMLRTCAVHITLDPDTANPWL |
| | | ILSEDRRQVRLGDTQQSIPGNEERFDSYPM |
| | | VLGAQHFHSGKHYWEVDVTGKEAWDLGVCR |
| | | DSVRRKGHFLLSSKSGFWTIWLWNKQKYEA |
| | | GTYPQTPLHLQVPPCQVGIFLDYEAGMVSF |
| | | YNITDHGSLIYSESECAFTGPLRPFFSPGF |
| | | NDGGKNTAPLTLCPLNIGSQGSTDY |

To establish functionality of the VA-DER TRIM21 systems, an ELISA is developed to demonstrate that TRIM21 binds to a full antibody sequence but not to a Fab2 sequence.

In such an assay, cell lysates (e.g., 293 cells) where the cells either express TRIM21 or do not express TRIM21 are prepared. Plates are coated with goat anti-mouse IgG or Fab2 goat anti-mouse IgG. TR1M21 or 293 cell lysate is then applied. Rabbit anti-TR1M21 polyclonal antibody is applied. Then HRP conjugated goat anti-rabbit secondary antibody is applied.

If HA-MB/121 is used, then BRP conjugated rabbit anti-HA is used. The readout is per any standard ELISA method. The assay demonstrates whether TRIM21 binds the test antibody and that the binding is effector function dependent.

Pull down assays are also used to illustrate the same outcome, i.e., that TRIM21 binds its target antibody.

### Neutralization Assay

Cell-based TRIM21 mediated antibody neutralization assays may also be used.

In such an assay, cells are prepared which express TRIM21. A GFP or other labeled AAV vector is prepared, for example AAV2, which expresses the label. A20, an antibody which recognizes a conformational epitope of AAV2 is incubated with the AAV2-GFP to determine if AAV2-GFP infection is impaired. If there is no impairment, the GFP levels may be measured and compared to levels in cells which do, or do not, express TRIM21. Controls are standard and include cells which do not express TRIM21 or which are not treated with antibody or GFP expressing vector. This assay shows if the TRIM21 in the cell is augmenting the A20 antibody bound AAV2 particle to the proteasome for destruction.

### VA-DER 1R1A 421 Assay

Vectored TRIM21 is evaluated for its ability to augment the destruction of an antibody of choice, and by extension any protein or antigen so bound by the antibody.

In this example, TRIM21 is delivered to a cell as an AAV payload. An antibody which is specific for a protein of interest, e.g., a cellular protein, is also delivered as an AAV payload.

The TRIM21, AAV and Antibody AAV are mixed in different ratios such as ft 1:1, 0.31, Li, L3 or 1:0 (TRIM21:Antibody). The AAV vectors are injected into a subject. In mice, the wt/P301s model is used. Levels of the protein targeted by the antibody encoded in the Antibody AAV are measured at a later time, e.g., 1 day, 1 week, 2, weeks, 3 weeks, 4 weeks or more by ELISA or TEC. Results demonstrate a TRIM21 dependent reduction in the protein targeted by the antibody. In some embodiments the protein being targeted is tau and the antibody is any tau binding antibody taught herein.

### VIII. Equivalents and Scope

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with reference to the appended claims so as to provide the broadest possible interpretation of such claims in view of the prior art and, therefore, to effectively encompass the intended scope of the disclosure.

LENGTHY TABLES
The patent application contains a lengthy table section. A copy of the table is available in electronic form from the USPTO web site (https://seqdata.uspto.gov/?pageRequest=docDetail&DocID=US20240124889A1). An electronic copy of the table will also be available from the USPTO upon request and payment of the fee set forth in 37 CFR 1.19(b)(3).

1. A vector-based system of augmenting protein destruction, expression and/or regulation comprising
a. a first component comprising a nucleic acid vector or plasmid sequence encoding a payload,
b. a second component comprising a TRIM21 protein or a nucleic acid sequence encoding a TRIM21 protein, and
c. optionally a third component comprising a biomolecule selected from the group consisting of a nucleic acid sequence, a protein sequence, a lipid, a small molecule and a vitamin.

2. The vector-based system of claim 1, wherein said first component is an AAV vector and the serotype of said AAV vector is selected from any of those listed in Table 1.

3. The vector-based system of claim 1, wherein said second component comprises a TRIM21 protein encoded in an AAV vector and the serotype of said AAV vector is selected from any of those listed in Table 1.

4. The vector-based system of claim 1, wherein the payload of said first component is an antibody.

5. The vector-based system of claim 4, wherein the antibody is selected from any of those listed in Tables 3-53.

6. The vector-based system of claim 1, wherein the payload is a chimeric antigen receptor.

7. A vector-based system of augmenting protein destruction, expression and/or regulation comprising
a. a first component comprising a nucleic acid vector or plasmid sequence encoding a payload, wherein the payload comprises a nucleic acid sequence encoding
i. at least one TRIM21 protein or TRIM21 protein fragment, and,
ii. at least one antibody or antibody fragment.

8. The vector-based system of claim 7, wherein said first component is an AAV vector and the serotype of said AAV vector is selected from any of those listed in Table 1.

9. The vector-based system of claim 7, wherein the antibody or antibody fragment is selected from any of those listed in Tables 3-53.

10. The vector-based system of claim 7, wherein the payload is a chimeric antigen receptor.

11. The vector-based system of claim 7, wherein the at least one antibody fragment is selected from the group consisting of an Fc, scFv, nanobody, intrabody, and Fab fragment or combinations thereof.

12. The vector-based system of claim 11, wherein the at least one antibody fragment is in combination with at least one other different antibody fragment.

13. The vector-based system of claim 12, wherein the at least one antibody fragment is an Fc fragment.

14. The vector-based system of claim 7, wherein the payload comprises a nucleic acid sequence further encoding
iii. at least one target binding protein or fragment thereof.

15. The vector-based system of claim 14, wherein the target binding protein is tau or a tau binding protein.

16. The vector-based system of claim 1, comprising a sequence selected from the group consisting of SEQ ID NO: 32672-32675.

17. An AAV viral genome comprising a sequence selected from the group consisting of SEQ ID NO: 32672-32675.

18. An AAV particle comprising the viral genome of claim 17 and a capsid.

19. A pharmaceutical composition comprising the AAV particle of claim 18.

20. A method of treating a disease, disorder, or condition in a subject, said method comprising administering to the subject the AAV particle of claim 18.

21. A method of treating a disease, disorder, or condition in a subject, said method comprising administering to the subject the pharmaceutical composition of claim 19.

22. The vector-based system of claim 7, comprising a sequence selected from the group consisting of SEQ ID NO: 32672-32675.
